(19) United States
(12) Patent Application Publication
(10) Pub. No.: US 2025/0302815 A1
(43) Pub. Date: Oct. 2, 2025

(54) PROTEIN TYROSINE PHOSPHATASE TARGETING LIGANDS

(71) Applicants: CALICO LIFE SCIENCES LLC, South San Francisco, CA (US); ABBVIE INC., North Chicago, IL (US)

(72) Inventors: Gesine Kerstin VEITS, Watertown, MA (US); Mark E. FITZGERALD, Watertown, MA (US); Alexander W. HIRD, Watertown, MA (US); James A. HENDERSON, Watertown, MA (US); Harit U. VORA, Watertown, MA (US); Ramzi F. SWEIS, North Chicago, IL (US); Michael E. KORT, North Chicago, IL (US); Mark MATULENKO, Stoneham, MA (US)

(21) Appl. No.: 18/682,858

(22) PCT Filed: Aug. 10, 2022
(86) PCT No.: PCT/US2022/074758

§ 371 Date: Feb. 9, 2024

Related U.S. Application Data
(60) Provisional application No. 63/231,646, filed on Aug. 10, 2021.
(60) Provisional application No. 63/290,235, filed on Dec. 16, 2021.

(51) Int. Cl. A61K 31/4545; A61P 35/00; C07D 409/14; C07D 413/14; C07D 471/04; C07D 471/10

(52) U.S. Cl. CPC A61K 31/4545; A61P 35/00; C07D 409/14; C07D 413/14; C07D 471/04; C07D 471/10

(57) ABSTRACT

Provided herein are compounds, compositions, and methods useful for degrading protein tyrosine phosphatase, e.g., protein tyrosine phosphatase non-receptor type 2 (PTPN2) and/or protein tyrosine phosphatase non-receptor type 1 (PTPN1), and for treating related diseases favorably responsive to PTPN1 or PTPN2 inhibitor treatment, e.g., a cancer or a metabolic disease.

### CROSS-REFERENCE TO RELATED APPLICATIONS

This application is a U.S. National Stage entry under 35 USC § 371 of the international application PCT/US2022/074758, filed Aug. 10, 2022, which claims the benefit of U.S. Provisional Application No. 63/290,235, filed Dec. 16, 2021, and U.S. Provisional Application No. 63/231,646, filed Aug. 10, 2021, the entirety of each of which is hereby incorporated by reference.

### BACKGROUND

Cancer immunotherapy regimens targeting immune evasion mechanisms including checkpoint blockade (e.g. PD-1/PD-L1 and CTLA-4 blocking antibodies) have been shown to be effective in treating in a variety of cancers, and dramatically improving outcomes in some populations refractory to conventional therapies. However, incomplete clinical responses and the development of intrinsic or acquired resistance continue to limit the subject populations who could benefit from checkpoint blockade.

Protein tyrosine phosphatase non-receptor type 2 (PTPN2), also known as T cell protein tyrosine phosphatase (TC-PTP), is an intracellular member of the class 1 subfamily of phospho-tyrosine specific phosphatases that control multiple cellular regulatory processes by removing phosphate groups from tyrosine substrates. PTPN2 is ubiquitously expressed, but expression is highest in hematopoietic and placental cells (Mosinger, B. Jr. et al., Proc Natl Acad Sci USA 89:499-503; 1992). In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif (Tillmann U. et al., Mol Cell Biol 14:3030-3040; 1994). The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g. JAK1, JAK3), receptor tyrosine kinases (e.g. INSR, EGFR, CSFIR, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1 (also called PTP1B), and shares similar enzymatic kinetics (Romsicki Y. et al., Arch Biochem Biophys 414:40-50; 2003).

Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade (Manguso R. T. et al., Nature 547:413-418; 2017). Loss of Ptpn2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTP1B), has been shown to play a key role in insulin and leptin signaling and is a primary mechanism for down-regulating both the insulin and leptin receptor signaling pathways (Kenner K. A. et al., J Biol Chem 271:19810-19816, 1996). Animals deficient in PTP1B have improved glucose regulation and lipid profiles and are resistant to weight gain when treated with a high fat diet (Elchebly M. et al., Science 283:1544-1548, 1999).

One approach to externally impact protein activity is by decreasing levels of a particular protein by targeted protein degradation. Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487); Berndsen et al. (Nat. Struct. Mol. Biol., 2014, 21, 301-307); Deshaies et al. (Ann. Rev. Biochem., 2009, 78, 399-434); Spratt et al. (Biochem. 2014, 458, 421-437); and Wang et al. (Nat. Rev. Cancer., 2014, 14, 233-347).

The first E3 ligase successfully targeted with a small molecule was SCF^{βTrCP}, using a hybrid of the small molecule MetAP2 inhibitor linked to a IκBα phosphopeptide epitope known to bind to the ubiquitin E3 ligase. (Sakamoto et al, PNAS 2001, 98 (15) 8554). Schneekloth et al. describe a degradation agent (PROTAC3) that targets the FK506 binding protein (FKBP12) and shows that both PROTAC2 and PROTAC3 hit their respective targets with green fluorescent protein (GFP) imaging. Schneekloth et al. (Chem Bio Chem 2005, 6, 40-46).

In unrelated parallel research, scientists were investigating thalidomide toxicity, and discovered that cereblon is a thalidomide binding protein. Ito et al. (Science 2010, 327, 1345-1350). Cereblon forms part of an E3 ubiquitin ligase protein complex which interacts with damaged DNA binding protein 1, forming an E3 ubiquitin ligase complex with Cullin 4 and the E2-binding protein ROC1 (also known as RBX1) where it functions as a substrate receptor to select proteins for ubiquitination. The study revealed that thalidomide-cereblon binding in vivo may be responsible for thalidomide teratogenicity. After the discovery that thalidomide binds to the cereblon E3 ubiquitin ligase led to research to investigate incorporating thalidomide and certain derivatives into compounds for the targeted destruction of proteins. See G. Lu et al., (Science, 343, 305-309 (2014)); and J. Kronke et al., (Science, 343, 301-305 (2014)).

While progress has been made in the area of modulation of the UPP for in vivo protein degradation, it would be useful to have additional compounds and approaches to more fully harness the UPP for therapeutic treatments, for example, for the development of targeted PTP1B degraders useful for the treatment of type 2 diabetes, obesity, and metabolic syndrome. It is an object of the present invention to provide new compounds, methods, compositions, and methods of manufacture that are useful to degrade selected proteins, e.g., PTP1B, in vivo.

### SUMMARY

The present disclosure is directed, at least in part, to compounds, compositions, and methods that cause degradation of a protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTP1B) via the ubiquitin proteasome pathway (UPP). In some embodiments, the compounds described herein comprise a “Targeting Ligand” that binds to a protein tyrosine phosphatase, a “Degron” which binds (e.g., non-covalently) to an E3 Ligase (e.g., the cereblon component) and a linker that covalently links the Targeting Ligand to the Degron.

Some embodiments provide a compound of Formula (I):

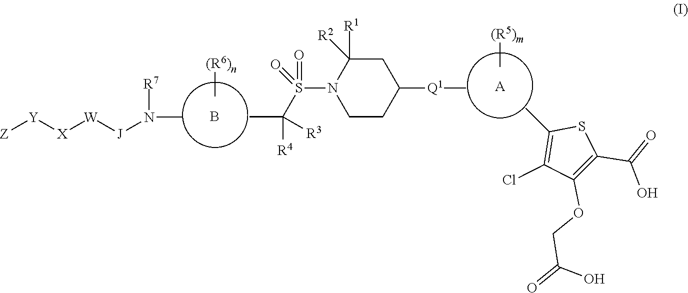

or a pharmaceutically acceptable salt thereof, wherein: R¹; R², R³, R⁴, R⁵; R⁶; R⁷; R^{8A}; R^{8B}; R⁹; R¹⁰, R¹¹; R¹²; R^{A}; R^{B}; R^{C}; R^{X}; R^{Y}; Ring A; Ring B; Q¹; J; W; X; Y; Y²; Z; p; p¹; p²; p³, q; s; and t are as defined herein.

Some embodiments provide a pharmaceutical composition comprising the compound of Formula (I), or a pharmaceutically acceptable salt thereof.

Some embodiments provide a method of treating cancer in a subject in need thereof, comprising administering to the subject a therapeutically effective amount of a compound of Formula (I), or a pharmaceutically acceptable salt thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

These and other features, aspects, and advantages of the present invention will become better understood with regard to the following description, and accompanying drawings, where:

FIG. 1 illustrates the anticancer and tumor growth inhibition activity in vivo of an exemplary compound of this disclosure in an syngeneic mouse tumor model. The graph shows tumor growth inhibition of MC-38 tumor-bearing mice treated with Compound 187b monotherapy (arrows labelled “3”; QWx3) and in combination with anti-PD-1 therapy (arrows labelled “2”; Q4Dx2). Each point on the curve represents the mean of 10 tumor volume of 10 mice. Error bars depict the standard error of the mean. **=p<0.01.

### BRIEF DESCRIPTION OF THE SEQUENCE LISTING

Incorporated herein by reference in its entirety is a Sequence Listing entitled, “31354-49432-US_002US”, comprising SEQ ID NO: 1 through SEQ ID NO: 3, which includes the amino acid sequences disclosed herein. The Sequence listing has been submitted herewith in xml format via EFS. The Sequence Listing was first created on May 20, 2020 and is 13 KB in size. It was subsequently converted from .txt to .xml in compliance with ST.26 on Mar. 26, 2024 and is 5,635 bytes in size.

### DETAILED DESCRIPTION

The present disclosure is directed, at least in part, to compounds, compositions, and methods for the inhibition of protein tyrosine phosphatase, e.g., protein tyrosine phosphatase non-receptor type 2 (PTPN2) and/or protein tyrosine phosphatase non-receptor type 1 (PTPN1 or PTP1B).

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers or double bonds, and thus can exist in various isomeric forms, e.g., enantiomers, diastereomers, racemates, geometric isomers, stereoisomeric forms that may be defined, in terms of absolute stereochemistry, as (R)- or (S)-. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure includes compounds in racemic and optically pure forms. When the compounds described herein contain olefinic bonds or other centers of geometric asymmetry, and unless specified otherwise, it is intended that the compounds include both E and Z geometric isomers. The disclosure additionally encompasses compounds described herein as individual isomers substantially free of other isomers, and alternatively, as mixtures of various isomers.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C1-C6 alkyl” is intended to encompass, C1, C2, C3, C4, C5, C6, C1-C6, C1-C5, C1-C4, C1-C3, C1-C2, C2-C6, C2-C5, C2-C4, C2-C3, C3-C6, C3-C5, C3-C4, C4-C6, C4-C5, and C5-C6 alkyl.

The following terms are intended to have the meanings presented therewith below and are useful in understanding the description and intended scope of the present disclosure.

“Alkyl” refers to a radical of a straight-chain or branched saturated hydrocarbon group having from 1 to 10 carbon atoms (“C1-C10 alkyl”). In some embodiments, an alkyl group has 1 to 8 carbon atoms (“C1-C8 alkyl”). In some embodiments, an alkyl group has 1 to 6 carbon atoms (“C1-C6 alkyl”). In some embodiments, an alkyl group has 1 to 5 carbon atoms (“C1-C5 alkyl”). In some embodiments, an alkyl group has 1 to 4 carbon atoms (“C1-C4 alkyl”). In some embodiments, an alkyl group has 1 to 3 carbon atoms (“C1-C3 alkyl”). In some embodiments, an alkyl group has 1 to 2 carbon atoms (“C1-C2 alkyl”). In some embodiments, an alkyl group has 1 carbon atom (“C1 alkyl”). In some embodiments, an alkyl group has 2 to 6 carbon atoms (“C2-C6 alkyl”). Examples of C1-C6 alkyl groups include methyl (C1), ethyl (C2), n-propyl (C3), isopropyl (C3), n-butyl (C4), tert-butyl (C4), sec-butyl (C4), iso-butyl (C4), n-pentyl (C5), 3-pentanyl (C5), amyl (C5), neopentyl (C5), 3-methyl-2-butanyl (C5), tertiary amyl (C5), and n-hexyl (C6). Additional examples of alkyl groups include n-heptyl (C7), n-octyl (C8) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C1-C10 alkyl (e.g., —CH₃). In certain embodiments, the alkyl group is substituted C1-C6 alkyl. Common alkyl abbreviations include Me (—CH₃), Et (—CH₂CH₃), iPr (—CH(CH₃)₂), nPr (—CH—CH₂CH₃), n-Bu (—CH₂CH₂CH₂CH₃), or i-Bu (—CH₂CH(CH₃)₂).

“Alkenyl” refers to a radical of a straight-chain or branched hydrocarbon group having from 2 to 10 carbon atoms, one or more carbon-carbon double bonds, and no triple bonds (“C2-C10 alkenyl”). In some embodiments, an alkenyl group has 2 to 8 carbon atoms (“C2-C8 alkenyl”). In some embodiments, an alkenyl group has 2 to 6 carbon atoms (“C2-C6 alkenyl”). In some embodiments, an alkenyl group has 2 to 5 carbon atoms (“C2-C5 alkenyl”). In some embodiments, an alkenyl group has 2 to 4 carbon atoms (“C2-C4 alkenyl”). In some embodiments, an alkenyl group has 2 to 3 carbon atoms (“C2-C3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-C4 alkenyl groups include ethenyl (C2), 1-propenyl (C3), 2-propenyl (C3), 1-butenyl (C4), 2-butenyl (C4), butadienyl (C4), and the like. Examples of C2-C6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Each instance of an alkenyl group may be independently optionally substituted, e.g., unsubstituted (an “unsubstituted alkenyl”) or substituted (a “substituted alkenyl”) with one or more substituents, e.g., from 1 to 5 substituents, 1 to 3 substituents, or 1 substituent. In certain embodiments, the alkenyl group is unsubstituted C2-C10 alkenyl. In certain embodiments, the alkenyl group is substituted C2-C6 alkenyl.

“Halo” or “halogen,” independently or as part of another substituent, means a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

“Haloalkyl” refers to an alkyl group as described herein (e.g., a C1-C6 alkyl group) in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl, and 2-fluoroisobutyl.

“Alkoxy” refers to an alkyl group as described herein (e.g., a C1-C6 alkyl group), which is attached to a molecule via oxygen atom. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

“Haloalkoxy” refers to an alkoxy group as described herein (e.g., a C1-C6 alkoxy group), in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkoxy, chloro-difluoroalkoxy, and 2-fluoroisobutoxy.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-C14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). An aryl group may be described as, e.g., a C6-C10 aryl. Aryl groups include, but are not limited to, phenyl, naphthyl, indenyl, and tetrahydronaphthyl. Each instance of an aryl group may be independently optionally substituted, e.g., unsubstituted (an “unsubstituted aryl”) or substituted (a “substituted aryl”) with one or more substituents. In certain embodiments, the aryl group is unsubstituted C6-C14 aryl. In certain embodiments, the aryl group is substituted C6-C14 aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Cycloalkyl” refers to a radical of a non-aromatic cyclic hydrocarbon group having from 3 to 10 ring carbon atoms (“C3-C10 cycloalkyl”) and zero heteroatoms in the non-aromatic ring system. In some embodiments, a cycloalkyl group has 3 to 8 ring carbon atoms (“C3-C8cycloalkyl”). In some embodiments, a cycloalkyl group has 3 to 6 ring carbon atoms (“C3-C6 cycloalkyl”). In some embodiments, a cycloalkyl group has 3 to 6 ring carbon atoms (“C3-C6 cycloalkyl”). In some embodiments, a cycloalkyl group has 5 to 10 ring carbon atoms (“C5-C10 cycloalkyl”). A cycloalkyl group may be described as, e.g., a C4-C7-membered cycloalkyl. Exemplary C3-C6 cycloalkyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-C8 cycloalkyl groups include, without limitation, the aforementioned C3-C6 cycloalkyl groups as well as cycloheptyl (C7), cycloheptenyl (C7), cycloheptadienyl (C7), cycloheptatrienyl (C7), cyclooctyl (C8), cyclooctenyl (C8), cubanyl (C8), bicyclo[1.1.1]pentanyl (C5), bicyclo[2.2.2]octanyl (C8), bicyclo[2.1.1]hexanyl (C6), bicyclo[3.1.1]heptanyl (C7), and the like. Exemplary C3-C10 cycloalkyl groups include, without limitation, the aforementioned C3-C8 cycloalkyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the cycloalkyl group is either monocyclic (“monocyclic cycloalkyl”) or contain a fused, bridged, or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is a substituted C3-C10 cycloalkyl.

In some embodiments, “cycloalkyl” is a monocyclic, saturated cycloalkyl group having from 3 to 10 ring carbon atoms (“C3-C10 cycloalkyl”). In some embodiments, a cycloalkyl group has 3 to 8 ring carbon atoms (“C3-C8 cycloalkyl”). In some embodiments, a cycloalkyl group has 3 to 6 ring carbon atoms (“C3-C6 cycloalkyl”). In some embodiments, a cycloalkyl group has 5 to 6 ring carbon atoms (“C5-C6 cycloalkyl”). In some embodiments, a cycloalkyl group has 5 to 10 ring carbon atoms (“C5-C10 cycloalkyl”). Examples of C5-C6 cycloalkyl groups include cyclopentyl (C5) and cyclohexyl (C5). Examples of C3-C6 cycloalkyl groups include the aforementioned C5-C6 cycloalkyl groups as well as cyclopropyl (C3) and cyclobutyl (C4). Examples of C3-C8 cycloalkyl groups include the aforementioned C3-C6 cycloalkyl groups as well as cycloheptyl (C7) and cyclooctyl (C8). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is substituted C3-C10 cycloalkyl.

“Heterocyclyl” refers to a radical of a 3- to 12-membered non-aromatic ring system having ring carbon atoms and 1 to 4 ring heteroatomic groups, wherein each heteroatomic group is independently selected from nitrogen, oxygen, sulfur and oxidized forms of sulfur (for example, S, S(O) and S(O)₂), boron, phosphorus, and silicon (“3-12 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, and sulfur and oxidized forms of sulfur (for example, S, S(O) and S(O)₂), within the moiety. Each instance of heterocyclyl may be independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-12 membered heterocyclyl. In certain embodiments, the heterocyclyl group is substituted 3-12 membered heterocyclyl. In certain embodiments, the heterocyclyl group is substituted 4-6 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” refers to the radical —NH₂.

“Cyano” refers to the radical —CN.

“Hydroxy” or “hydroxyl” refers to the radical —OH.

“Oxo” refers to a ═O group.

In some embodiments one or more of the nitrogen atoms of a disclosed compound if present are oxidized to the corresponding N-oxide.

The term “pharmaceutically acceptable salts” is meant to include salts that are prepared with relatively nontoxic acids or bases, depending on the particular substituents found on the compounds described herein.

Certain compounds described herein can exist in unsolvated forms as well as solvated forms, including hydrated forms. In general, the solvated forms are equivalent to unsolvated forms and are encompassed within the scope of the present disclosure.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the invention, and the naming of the compounds does not exclude any tautomer. An example of a tautomeric forms includes the following example:

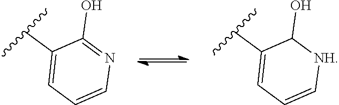 [SMILES: *c1cccnc1O ; *C1=CC=CNC1O]

It will be apparent to one skilled in the art that certain compounds of this disclosure may exist in tautomeric forms, all such tautomeric forms of the compounds being within the scope of the disclosure.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (H). As some of the aforementioned isotopes are radioactive. the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as additional agents, e.g., therapeutic agents, research reagents, e.g., assay reagents, and diagnostic agents, e.g., in vivo imaging agents. All isotopic variations of the compounds provided herein, whether radioactive or not, are intended to be encompassed within the scope of the present invention. For example, in some embodiments, one or more C—H groups in the naphthyl ring shown in Formula (I) are replaced with C-D groups.

In the compounds described herein, it is understood that the W—X—Y group does not include compounds, for example, where X is a bond and W and Y are both heteroatoms (e.g., W and Y are both-((CR^{A}R^{B})pO)t-*, where p is 0 and t is 1). Likewise, W and Y groups do not include compounds with multiple heteroatom-heteroatom bonds for example, when W and/or Y is —((CR^{A}R^{B})pO)t-*, where p is 0 and t is 2 or 3).

“Treating” or “treatment” refers to reducing the symptoms or arresting or inhibiting further development of the disease (in whole or in part). “Treating” or “treatment” includes any effect, e.g., lessening, reducing, modulating, or eliminating, that results in the improvement of the disease and the like. For example, certain methods herein treat cancer by decreasing or reducing the occurrence, growth, metastasis, or progression of cancer or decreasing a symptom of cancer.

An “effective amount” is an amount sufficient to accomplish a stated purpose (e.g. achieve the effect for which it is administered, treat a disease, reduce enzyme activity, increase enzyme activity, or reduce one or more symptoms of a disease). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of a disease, or reducing the likelihood of the onset (or reoccurrence) of a disease or its symptoms.

A “reduction” of a symptom or symptoms means decreasing of the severity or frequency of the symptom(s), or the complete elimination of the symptom(s).

“Contacting” refers to the process of allowing at least two distinct species to become sufficiently proximal to react, interact, and/or physically touch. It should be appreciated, however, that the resulting reaction product can be produced directly from a reaction between the added reagents or from an intermediate from one or more of the added reagents which can be produced in the reaction mixture. The term “contacting” includes allowing two species to react, interact, and/or physically touch, wherein the two species may be a compound as described herein and a protein or enzyme, e.g., a protein tyrosine phosphatase, e.g., protein tyrosine phosphatase non-receptor type 2 (PTPN2) or protein tyrosine phosphatase non-receptor type 1 (PTP1B).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction in the progression of a disease and/or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a protein tyrosine phosphatase, e.g., protein tyrosine phosphatase non-receptor type 2 (PTPN2) or protein tyrosine phosphatase non-receptor type 1 (PTP1B). Thus, inhibition may include, at least in part, partially or totally decreasing stimulation, decreasing or reducing activation, or inactivating, desensitizing, or down-regulating signal transduction or enzymatic activity or the amount of a protein tyrosine phosphatase, e.g., protein tyrosine phosphatase non-receptor type 2 (PTPN2) or protein tyrosine phosphatase non-receptor type 1 (PTP1B).

A “subject,” as used herein, refers to a living organism suffering from or prone to a disease that can be treated by administration of a compound or pharmaceutical composition, as provided herein. Non-limiting examples include mammals such as humans. In some embodiments, a subject is human. In some embodiments, a subject is a newborn human. In some embodiments, a subject is an elderly human. In some embodiments, the subject is a pediatric subject (e.g., a subject 21 years of age or less).

“Disease” refers to a state of being or health status of a subject or subject capable of being treated with a compound, pharmaceutical composition, or method provided herein. In some embodiments, the compounds and methods described herein comprise reduction or elimination of one or more symptoms of the disease, e.g., through administration of a compound described herein, a pharmaceutically acceptable salt thereof, or a pharmaceutical composition comprising a compound described herein, or a pharmaceutically acceptable salt thereof.

The term “PTPN2” as used herein refers to protein tyrosine phosphatase non-receptor type 2.

The term “PTPN1” refers to protein tyrosine phosphatase non-receptor type 1 (PTPN1), also known as protein tyrosine phosphatase-1B (PTP1B).

### Compounds

Some embodiments provide a compound of Formula (I):

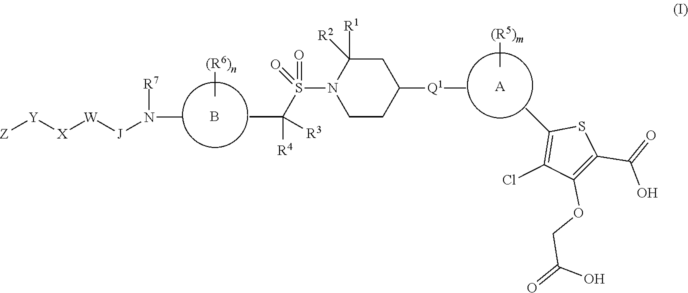

or a pharmaceutically acceptable salt thereof:
wherein:
   R¹ and R² are independently hydrogen or C1-C6 alkyl; or R¹ and R², together with the carbon atom to which they are attached, come together to form a C3-C4 cycloalkyl or a 3-4 membered heterocyclyl;
   R³ and R⁴ are independently hydrogen, C1-C6 alkyl, or phenyl; or R³ and R⁴, together with the carbon atom to which they are attached, come together to form a C3-C4 cycloalkyl or a 3-4 membered heterocyclyl;
   each R⁵ and R⁶ is independently halogen, C1-C6 alkyl, C1-C6 alkoxy, C1-C6 haloalkyl, C1-C6 haloalkoxy, hydroxyl, or —NR^{8A}R^{8B};
   R⁷ is hydrogen, C1-C6 alkyl, or C3-C4 cycloalkyl; or wherein R⁷ and R^{C} of W, together with the nitrogen atoms to which they are attached, come together to form a 5-6 membered heterocyclyl;
   R^{8A} and R^{8B} are independently hydrogen or C1-C6 alkyl;
   Ring A and Ring B are independently phenyl or 6-membered heteroaryl;
   n and m are independently 0, 1, or 2;
   Q¹ is O or NR⁹;
   R⁹ is hydrogen or C1-C6 alkyl;
   J is a bond or —C(═O)—;
   W and Y are independently-(CR^{A}R^{B})p-*, —((CR^{A}R^{B})pO)t-*, —(O(CR^{A}R^{B})p)t-*, —((CR^{A}R^{B})pO)t-(CR^{A}R^{B})p-*, —((CR^{A}R^{B})₂O(CR^{A}R^{B})₂))p-*, —NR^{C}(CR^{A}R^{B})p-*, —(CR^{A}R^{B})pNR^{C}(C═O)(CR^{A}R^{B})p-*, —(CR^{A}R^{B})p(C═O)NR^{C}(CR^{A}R^{B})p-*, —(CR^{A}R^{B})pNR^{C}-*, —(CR^{A}R^{B})pNR^{C}(C—O)(CR^{A}R^{B})p-O—*, —(CR^{A}R^{B})p(C═O)NR^{C}(CR^{A}R^{B})s-O—*, —(CR^{A}R^{B})pNR^{C}(C═O)(CR^{A}R^{B})p-NR^{C}—*, —(CR^{A}R^{B})p(C═O)NR^{C}(CR^{A}R^{B})s-NR^{C}—*; —NR^{C}(CR^{A}R^{B})s-NR^{C}(C═O)(CR^{A}R^{B})p-*, —NR^{C}(CR^{A}R^{B})p)C≡C—*, —C≡C(CR^{A}R^{B})p)NR^{C}—*, —(C═O)(CR^{A}R^{B})p-O—*, —O—*, —O—(CR^{A}R^{B})p-(C═O)—*, —(C═O)(CR^{A}R^{B})p-NR^{C}—*, —NR^{C}—(CR^{A}R^{B})p-(C═O)—*, —(C═O)((CR^{A}R^{B})p)-*, —(C═O)(CR^{A}R^{B})p-O—(CR^{A}R^{B})p-*, or —((CR^{A}R^{B})p)(C═O)—*, wherein the asterisk represents the point of attachment of W to X and the point of attachment of Y to Z;
   each p is independently 0, 1, 2, 3, 4, or 5;
   each s is independently 2, 3, 4, or 5;
   each t is independently 1, 2, or 3;
   each R^{A} and R^{B} are independently hydrogen, fluoro, or C1-C6 alkyl; or R^{A} and R^{B}, together with the carbon atom to which they are attached, come together to form a C3-C4 cycloalkyl;
   each R^{C} is independently hydrogen or C1-C6 alkyl; or wherein R^{C} of W and R⁷, together with the nitrogen atoms to which they are attached, come together to form a 5-6 membered heterocyclyl;
   X is a bond, C3-C6 cycloalkyl, phenyl optionally substituted with 1-3 independently selected halogen atoms, 3 to 10 membered heterocyclyl optionally substituted with 1-3 substituents independently selected from halogen, C1-C6 alkyl, and hydroxyl, or 5 to 10 membered heteroaryl optionally substituted with 1-3 independently selected halogen;
   Z is selected from the group consisting of

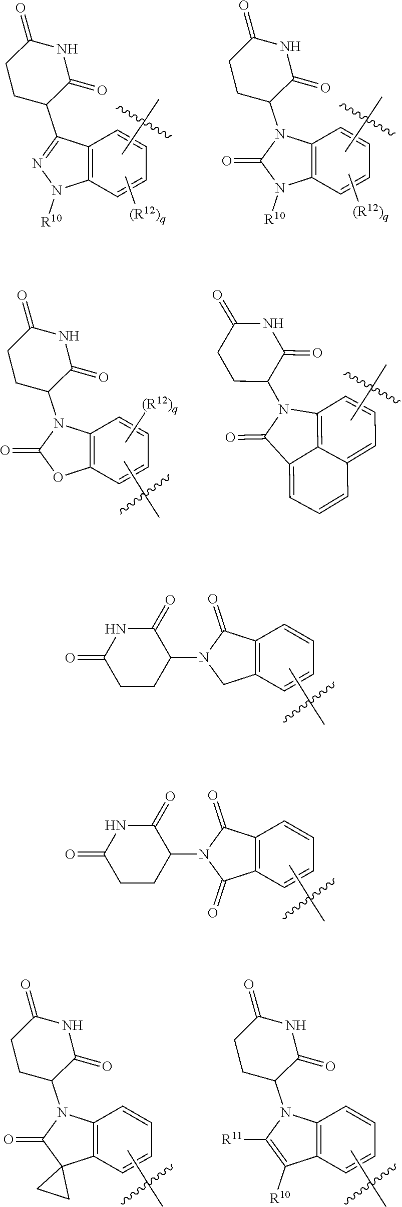

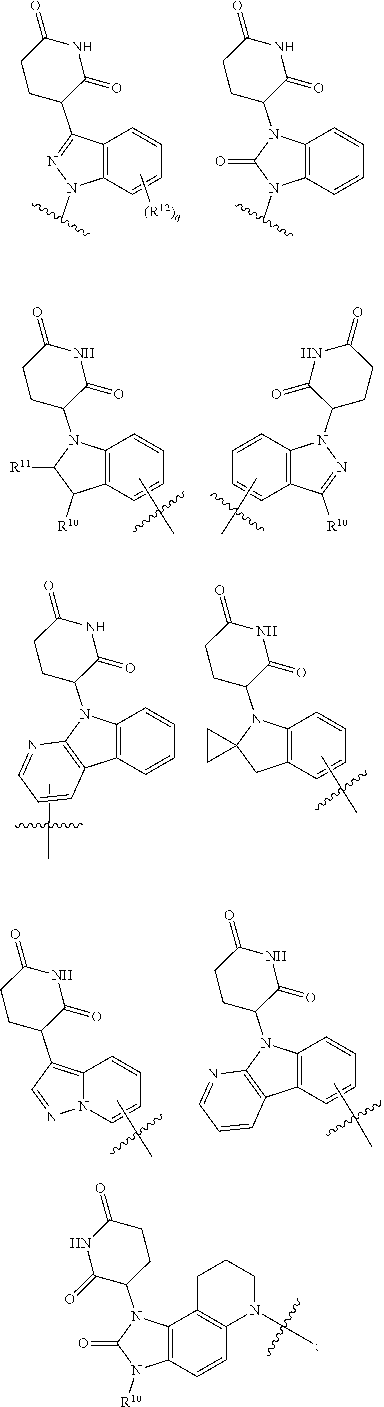

R¹⁰ is hydrogen, C1-C6 alkyl optionally substituted with C1-C6 alkoxy, C1-C6 haloalkyl, C3-C6 cycloalkyl, or 4-6 membered heterocyclyl; and
R¹¹ is hydrogen or C1-C6 alkyl;
each R¹² is independently halogen, cyano, C1-C6 alkyl, C1-C6 haloalkyl, C1-C6 alkoxy, or C3-C5 cycloalkoxy; and
q is 0, 1, or 2.

In some embodiments, Ring A is phenyl. In some embodiments, Ring A is:

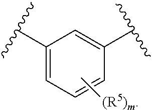

In some embodiments, m is 1. In some embodiments, R⁵ is halogen. In some embodiments, R⁵ is —F.

In some embodiments, m is 0.

In some embodiments, Q¹ is NR⁹. In some embodiments, R⁹ is hydrogen. In some embodiments, Q¹ is —O—.

In some embodiments, R¹ and R² are independently hydrogen or C1-C6 alkyl. In some embodiments, R¹ and R² are independently hydrogen or C1-C3 alkyl. In some embodiments, R¹ and R² are both hydrogen. In some embodiments, R¹ and R² are independently a C1-C3 alkyl. In some embodiments, R¹ and R² are both methyl.

In some embodiments, R³ and R⁴ are independently hydrogen, C1-C6 alkyl, or phenyl. In some embodiments, R³ and R⁴ are both hydrogen.

In some embodiments, Ring B is phenyl. In some embodiments, Ring B is:

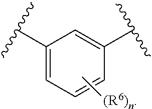

In some embodiments, n is 1. In some embodiments, R⁶ is halogen. In some embodiments, R⁶ is —F.

In some embodiments, n is 0.

In some embodiments, R⁷ is hydrogen or C1-C3 alkyl. In some embodiments, R¹ is hydrogen.

In some embodiments, W is —(CR^{A}R^{B})p-*. In some embodiments, W is —((CR^{A}R^{B})pO)t-*, —((CR^{A}R^{B})pO)t-(CR^{A}R^{B})p-*, or —((CR^{A}R^{B})₂O(CR^{A}R^{B}) 2)p-*. In some embodiments, W is (CR^{A}R^{B})pO-*, —O(CR^{A}R^{B})p-*, or —(CR^{A}R^{B})pNR^{C}-*.

In some embodiments. Y is —(CR^{A}R^{B})p-*. In some embodiments, Y is —(C═O)(CR^{A}R^{B})p-O—*, —(C═O)(CR^{A}R^{B})p-NR^{C}—*, or —(C═O)(CR^{A}R^{B})p-*. In some embodiments, Y is —(C═O)(CR^{A}R^{B})p-NR^{C}—*.

In some embodiments, Y is —(CR^{A}R^{B})p(C═O)NR^{C}(CR^{A}R^{B})p-*, —(CR^{A}R^{B})pNR^{C}(C═O)(CR^{A}R^{B})p-*, —(CR^{A}R^{B})pNR^{C}(C═O)(CR^{A}R^{B})pO—*, —(CR^{A}R^{B})pNR^{C}(C═O)(CR^{A}R^{B})p-NR^{C}—*, or —C≡C((CR^{A}R^{B})p)NR^{C}—*.

In some embodiments, Y is —(CR^{A}R^{B})pO—*, —O(CR^{A}R^{B})p-*, or —(CR^{A}R^{B})pNR^{C}-*.

In some embodiments, R^{C} of W and R⁷, together with the nitrogen atoms to which they are attached, come together to form a 5-6 membered heterocyclyl. In some embodiments, the 5-6 membered heterocyclyl is an imidazoline-2-one or a tetrahydropyrimidine-2 (1H)-one.

In some embodiments, each p is independently 0, 1, or 2. In some embodiments, one or more p is 0. In some embodiments, one or more p is 1 or 2. In some embodiments, one or more p is 1. In some embodiments, one or more p is 2. In some embodiments, one p is 3, 4, or 5; and each remaining p, if present, is independently 0, 1, or 2. In some embodiments, one p is 3, 4, or 5; and each remaining p, if present, is 0. In some embodiments, one p is 3, 4, or 5; and each remaining p, if present, is 1. In some embodiments, one p is 3, 4, or 5; and each remaining p, if present, is 2. In some embodiments, each p is 1. In some embodiments, each p is 2.

In some embodiments, R^{A} and R^{B} are independently hydrogen, fluoro, or C1-C3 alkyl. In some embodiments, R^{A} and R^{B} are both hydrogen. In some embodiments, from 1-2 R^{A} and/or R^{B} is fluoro or C1-C3 alkyl; and each remaining R^{A} and/or R^{B} is hydrogen.

In some embodiments, X is 3 to 10 membered heterocyclyl optionally substituted with 1-3 substituents independently selected from halogen, C1-C6 alkyl, and hydroxyl. In some embodiments, X is 4-6 membered monocyclic heterocyclyl optionally substituted with 1-3 substituents independently selected from halogen, C1-C6 alkyl, and hydroxyl. In some embodiments. X is azetidinyl, piperidinyl, or piperazinyl optionally substituted with 1-3 substituents independently selected from halogen, C1-C6 alkyl, and hydroxyl. In some embodiments, X is

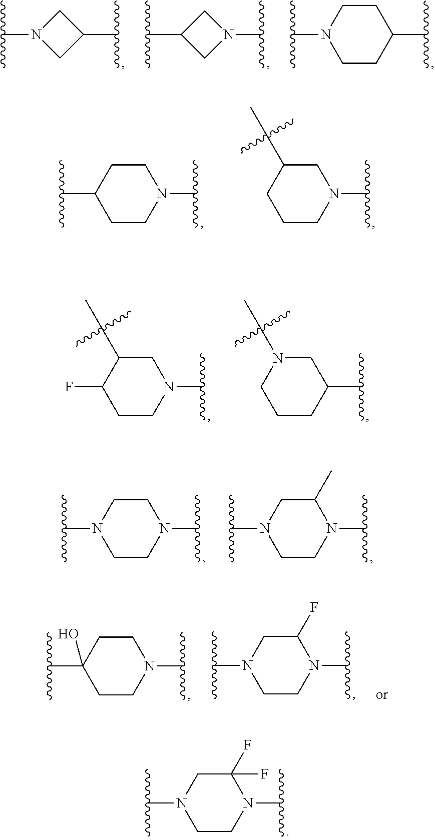

In some embodiments, X is selected from the group consisting of

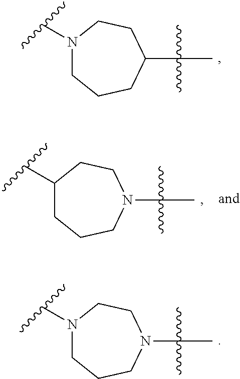 [SMILES: *C1CCCN(*)CC1 ; *C1CCCN(*)CC1 ; *N1CCCN(*)CC1]

In some embodiments, X is 6-10 membered bicyclic heterocyclyl. In some embodiments, X is 6-10 membered bicyclic fused heterocyclyl. In some embodiments, X is 7-10 membered bicyclic spiroheterocyclyl. In some embodiments, X is

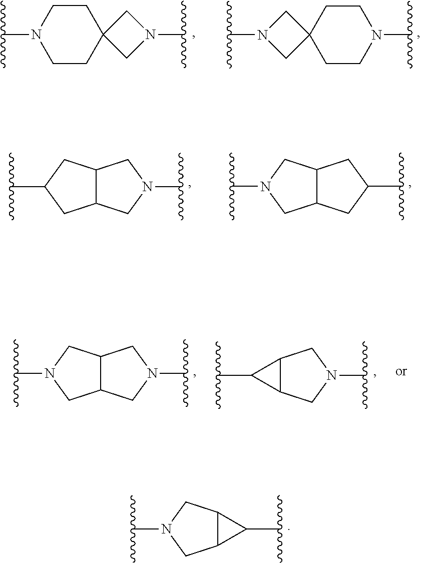

In some embodiments, X is C3-C6 cycloalkyl. In some embodiments, X is cyclohexyl.

In some embodiments, X is phenyl or 5 to 10 membered heteroaryl, wherein each is optionally substituted with 1-3 independently selected halogen. In some embodiments, X is phenyl optionally substituted with 1-3 independently selected halogen. In some embodiments, X is selected from the groups consisting of

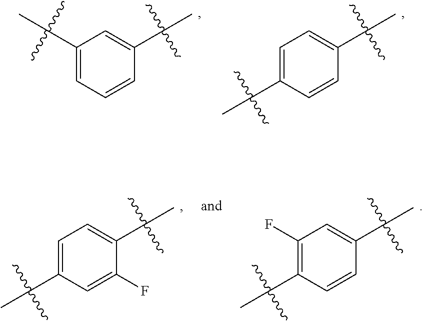 [SMILES: *c1cccc(*)c1 ; *c1ccc(*)cc1 ; *c1ccc(*)c(F)c1 ; *c1ccc(*)c(F)c1]

In some embodiments, X is 5-6 membered heteroaryl. In some embodiments, X is 1,2,3-triazolyl, pyrazolyl, or imidazolyl. In some embodiments, X is selected from the group consisting of

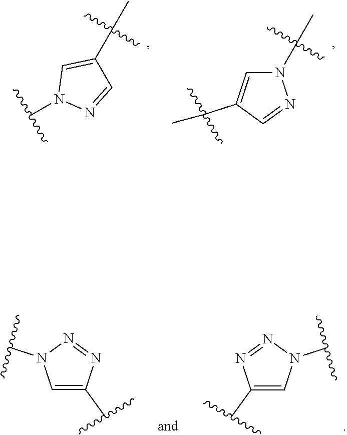 [SMILES: *c1cnn(*)c1 ; *c1cnn(*)c1 ; *c1cn(*)nn1 ; *c1cn(*)nn1]

In some embodiments, X is a bond.

In some embodiments, J is a bond.

In some embodiments, J is-C(═O)—.

In some embodiments, X is 4-10 membered heterocyclyl; and W is —(CR^{A}R^{B})p¹-*. In some embodiments, X is 4-10 membered heterocyclyl; and W is —(CR^{A}R^{B})p¹O—*, —O(CR^{A}R^{B})p¹-*, or —(CR^{A}R^{B})p¹-NR^{C}—*.

In some embodiments, Y is —(CR^{A}R^{B})p²-*. In some embodiments, Y is —(C═O)(CR^{A}R^{B})p²-O—*, (C═O)(CR^{A}R^{B})p²-NR^{C}—*, or —(C═O)(CR^{A}R^{B})p²-*. In some embodiments, Y is —(C═O)(CR^{A}R^{B})p²-NR^{C}—*. In some embodiments, Y is —(CR^{A}R^{B})p²-O—*, —O(CR^{A}R^{B})p²-*, —NR^{C}—(CR^{A}R^{B})p²-*, or —(CR^{A}R^{B})p²-NR^{C}—*.

In some embodiments, X is a bond; and W is —((CR^{A}R^{B})pO)t-*. In some embodiments, —((CR^{A}R^{B})pO)t-* is —((CR^{A}R^{B})₂O)t-*. In some embodiments, ((CR^{A}R^{B})pO)t-* is —((CR^{A}R^{B})₂O)t-*.

In some embodiments, Y is —(CR^{A}R^{B})p²-*. In some embodiments, Y is —(C═O)(CR^{A}R^{B})p²-O—*, —(C═O)(CR^{A}R^{B})p²-NR^{C}—*, or —(C═O)(CR^{A}R^{B})p²-*. In some embodiments, Y is —(CR^{A}R^{B})p² (C═O)NR^{C}(CR^{A}R^{B})p³-*, —(CR^{A}R^{B})p²-NR^{C}(C═O)(CR^{A}R^{B})p³-*, —(CR^{A}R^{B})p²-NR^{C}(C═O)(CR^{A}R^{B})p³-O—*, or —(CR^{A}R^{B})p²-NR^{C}(C═O)(CR^{A}R^{B})p³-NR^{C}—*.

In some embodiments, p¹ is 0, 1, 2, 3, 4, or 5. In some embodiments, p¹ is 0. In some embodiments, p¹ is 1. In some embodiments, p¹ is 2. In some embodiments, p¹ is 3. In some embodiments, p¹ is 4. In some embodiments, p¹ is 5.

In some embodiments, p² is 0, 1, 2, 3, 4, or 5. In some embodiments, p² is 0. In some embodiments, p² is 1. In some embodiments, p² is 2. In some embodiments, p² is 3. In some embodiments, p² is 4. In some embodiments, p² is 5.

In some embodiments, p³ is 0, 1, 2, 3, 4, or 5. In some embodiments, p³ is 0. In some embodiments, p³ is 1. In some embodiments, p² is 2. In some embodiments, p³ is 3. In some embodiments, p³ is 4. In some embodiments, p³ is 5.

In some embodiments, p¹ and p² are independently 0, 1, 2, 3, 4, or 5. In some embodiments, p² and p³ are independently 0, 1, 2, 3, 4, or 5.

In some embodiments, p¹+p²=0. In some embodiments, p¹+p²=1 or 2. In some embodiments, p¹+p²=1. In some embodiments, p¹+p²=2. In some embodiments, p¹+p²≤3. In some embodiments, p¹+p²=3. In some embodiments, p¹+p²=4. In some embodiments, p¹+p³=5. In some embodiments, p¹+p²=6. In some embodiments, p¹+p²=7. In some embodiments, p¹+p²=8. In some embodiments, p¹+p²=9. In some embodiments, p¹+p²=10. In some embodiments, p¹+p²=4, 5, 6, 7, 8, or 9.

In some embodiments, p²+p³=0. In some embodiments, p²+p³=1 or 2. In some embodiments, p²+p³=1. In some embodiments, p²+p³=2. In some embodiments, p²+p³≤3. In some embodiments, p²+p³=3. In some embodiments, p²+p³=4. In some embodiments, p²+p³=5. In some embodiments, p²+p³=6. In some embodiments, p²+p³=7. In some embodiments, p²+p³=8. In some embodiments, p²+p³=9. In some embodiments, p²+p³=10. In some embodiments, p²+p³=4, 5, 6, 7, 8, or 9.

In some embodiments, t is 1, 2, or 3. In some embodiments, t is 1. In some embodiments, t is 2. In some embodiments, t is 3.

In some embodiments, t+p²=3, 4, 5, or 6. In some embodiments, t+p²=3. In some embodiments, t+p²=4. In some embodiments, t+p²=5. In some embodiments, t+p²=6.

In some embodiments, t+p²+p³=3, 4, 5, or 6. In some embodiments, t+p²+p³=3. In some embodiments, t+p²+p³=4. In some embodiments, t+p²+p³=5. In some embodiments, t+p²+p³=6.

In some embodiments, R^{A} and R^{B} are independently hydrogen, fluoro, or C1-C3 alkyl. In some embodiments, R^{A} and R^{B} are both hydrogen.

In some embodiments, q is 0. In some embodiments, q is 1. In some embodiments, q is 2.

In some embodiments, Z is

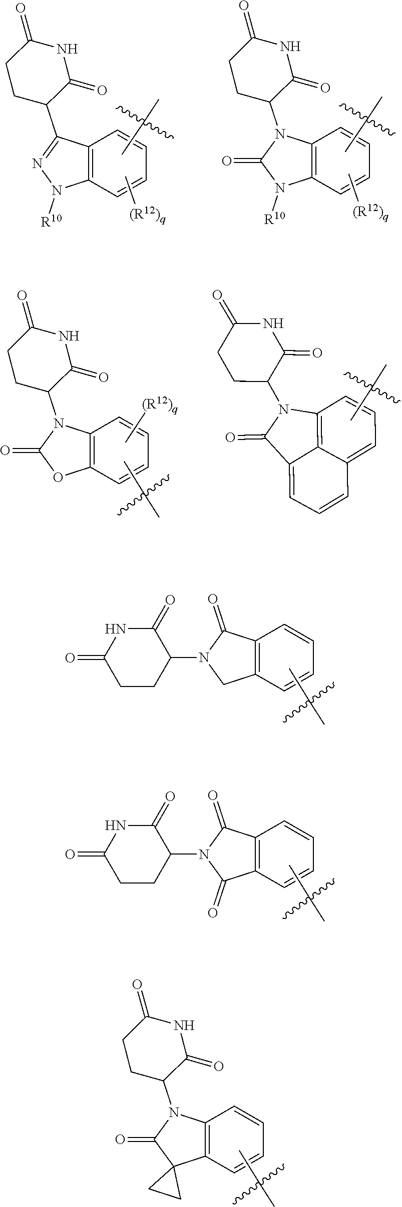

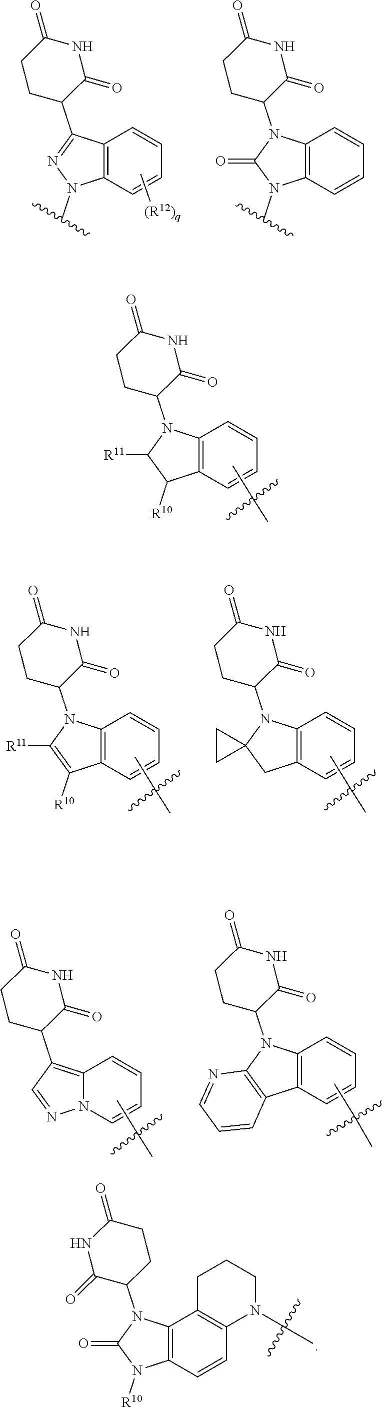

In some embodiments, Z is

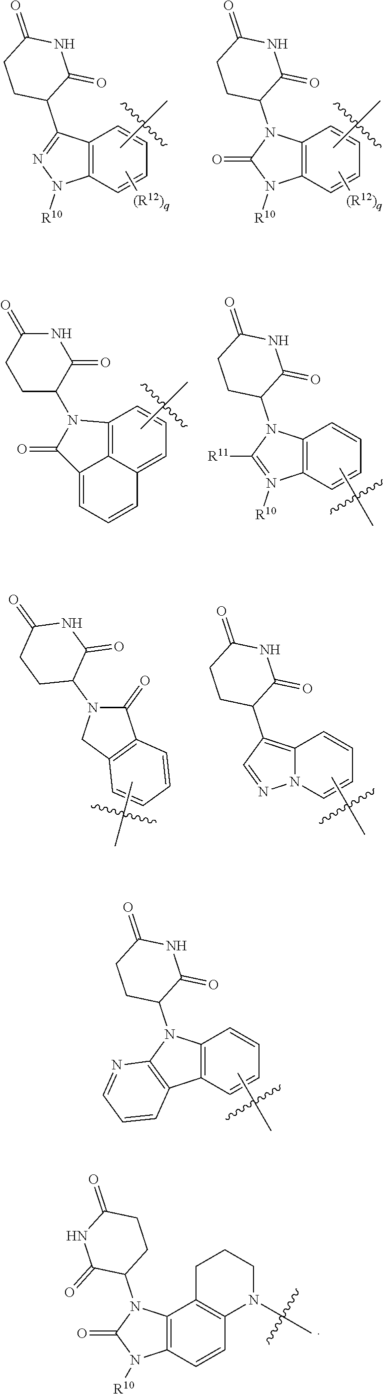

In some embodiments, Z is

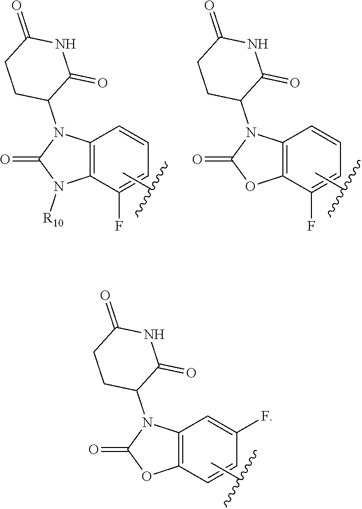

In some embodiments, Z is

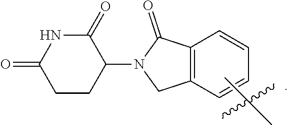

In some embodiments, Z is

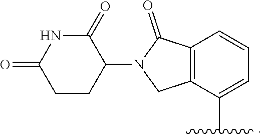 [SMILES: *c1cccc2c1CN(C1CCC(=O)NC1=O)C2=O]

In some embodiments, Z is

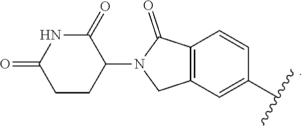 [SMILES: *c1ccc2c(c1)CN(C1CCC(=O)NC1=O)C2=O]

In some embodiments, Z is

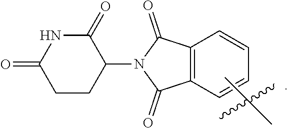

In some embodiments, Z is

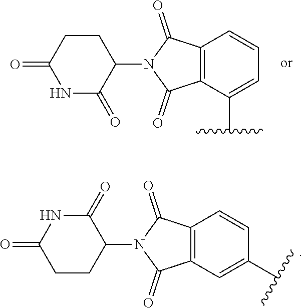 [SMILES: *c1cccc2c1C(=O)N(C1CCC(=O)NC1=O)C2=O ; *c1ccc2c(c1)C(=O)N(C1CCC(=O)NC1=O)C2=O]

In some embodiments, Z is

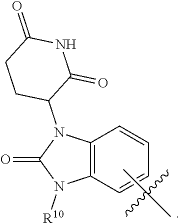

In some embodiments, Z is

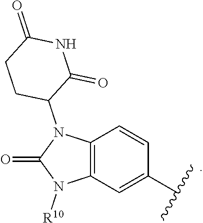

In some embodiments, Z is

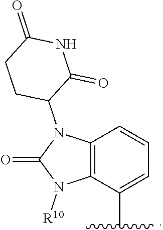

In some embodiments, Z is

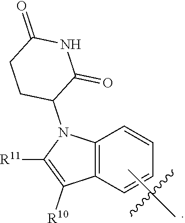

In some embodiments, Z is

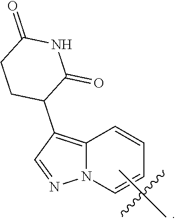

In some embodiments, Z is

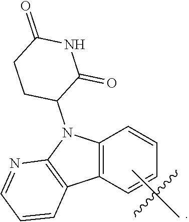

In some embodiments, Z is

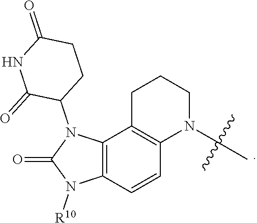

In some embodiments, Z is

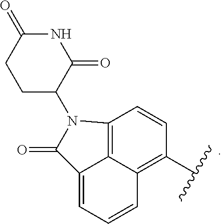 [SMILES: *c1ccc2c3c(cccc13)C(=O)N2C1CCC(=O)NC1=O]

In some embodiments, Z is

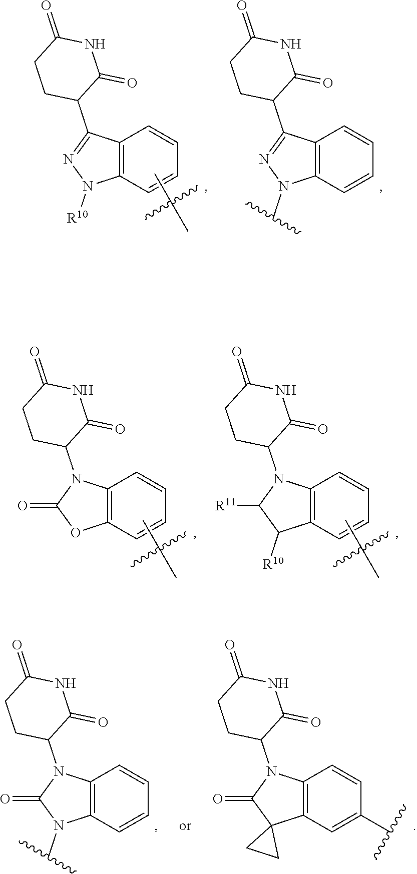

In some embodiments, R¹⁰ is C1-C4 alkyl optionally substituted with C1-C6 alkoxy. In some embodiments, R¹⁰ is methyl, ethyl, isopropyl, or t-butyl. In some embodiments, R¹⁰ is —(CH₂)₂OCH₃. In some embodiments, R¹⁰ is 3-oxetanyl.

In some embodiments, the compound of Formula (I), or a pharmaceutically acceptable salt thereof, is a compound of Formula (I-a):

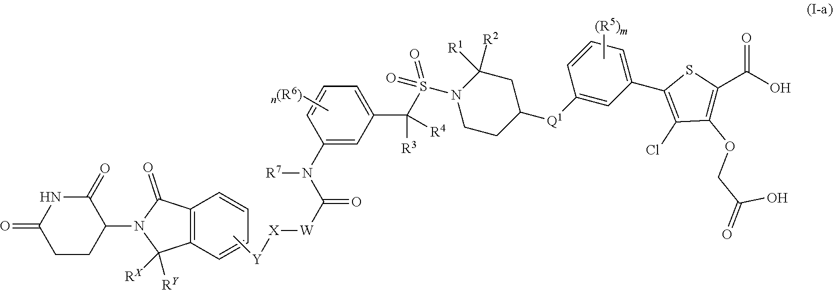

or a pharmaceutically acceptable salt thereof;
wherein R^{X} and R^{Y} are both H; or R^{X} and R^{Y}, together with the carbon atom to which they are attached, combine to form C═O.

In some embodiments, the compound of Formula (I), or a pharmaceutically acceptable salt thereof, is a compound of Formula (I-a1):

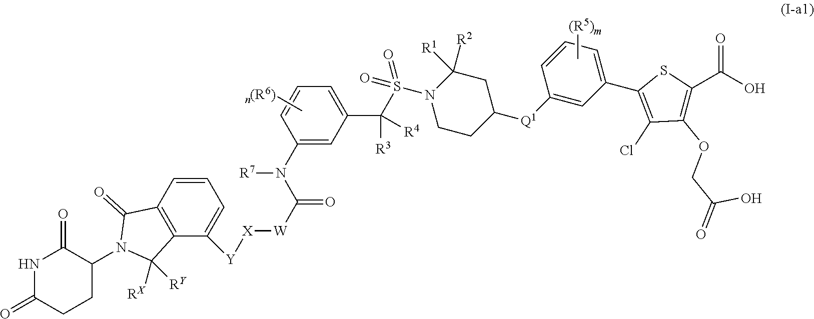

or a pharmaceutically acceptable salt thereof.

In some embodiments, the compound of Formula (I), or a pharmaceutically acceptable salt thereof, is a compound of Formula (I-a2):

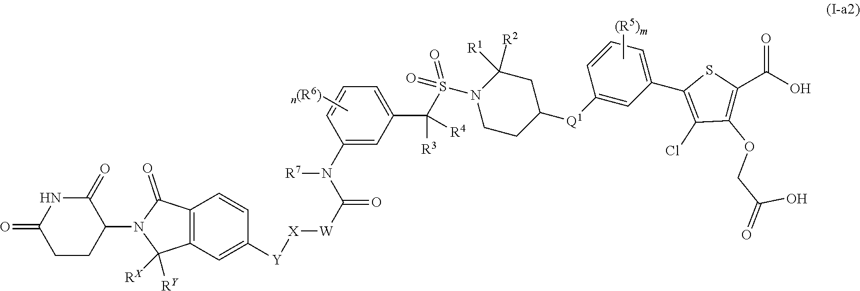

or a pharmaceutically acceptable salt thereof.

In some embodiments, both R^{X} and R^{Y} are hydrogen. In some embodiments, R^{X} and R^{Y}, together with the carbon atom to which they are attached, combine to form C═O.

In some embodiments, the compound of Formula (I), or a pharmaceutically acceptable salt thereof, is a compound of Formula (I-b):

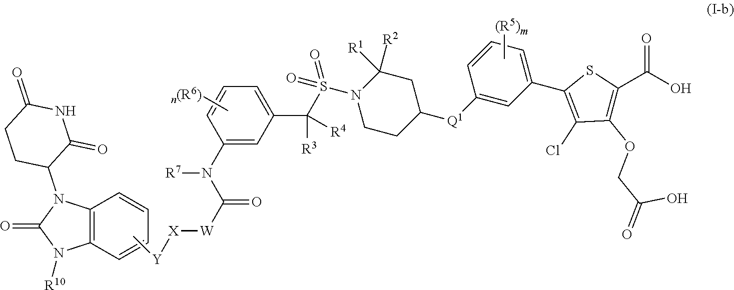

or a pharmaceutically acceptable salt thereof,
wherein R¹⁰ is hydrogen, C1-C4 alkyl optionally substituted with C1-C6 alkoxy, C1-C6 haloalkyl, C3-C6 cycloalkyl, or 4-6 membered heterocyclyl.

In some embodiments, the compound of Formula (I), or a pharmaceutically acceptable salt thereof, is a compound of Formula (I-b1):

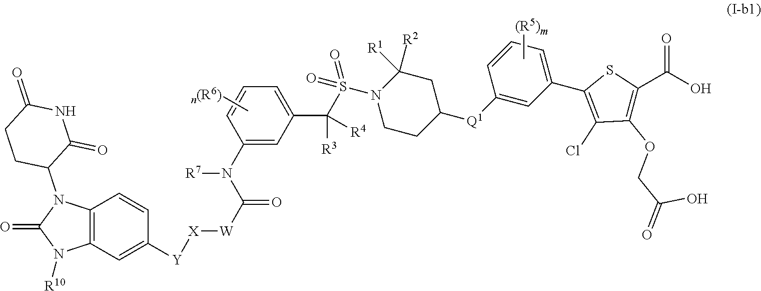

or a pharmaceutically acceptable salt thereof;
wherein R¹⁰ is methyl, ethyl, or isopropyl.

In some embodiments, the compound of Formula (I), or a pharmaceutically acceptable salt thereof, is a compound of Formula (I-b2):

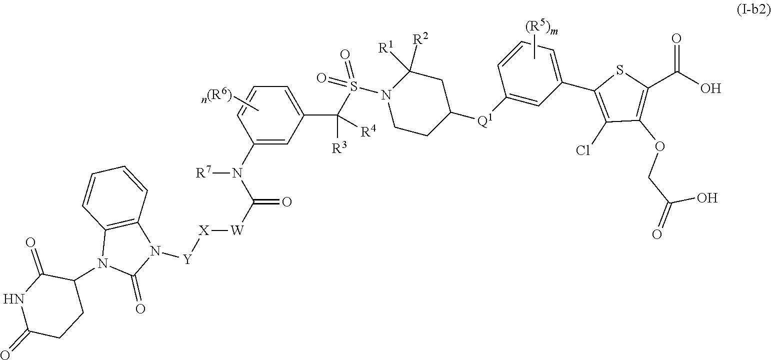

or a pharmaceutically acceptable salt thereof.

In some embodiments, the compound of Formula (I), or a pharmaceutically acceptable salt thereof, is a compound of Formula (I-b3):

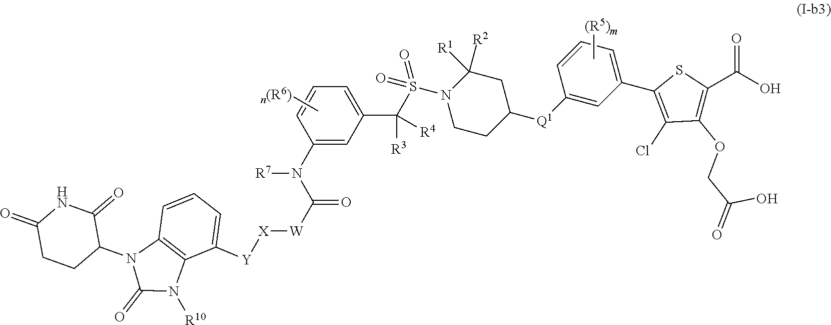

or a pharmaceutically acceptable salt thereof.

In some embodiments, the compound of Formula (I), or a pharmaceutically acceptable salt thereof, is a compound of Formula (I-c):

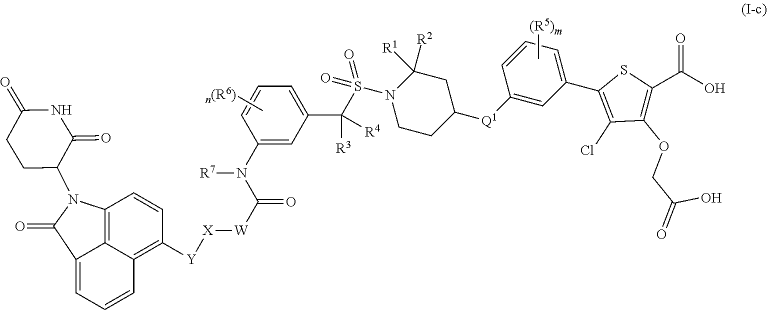

or a pharmaceutically acceptable salt thereof.

In some embodiments, the compound of Formula (I), or a pharmaceutically acceptable salt thereof, is a compound of Formula (I-d):

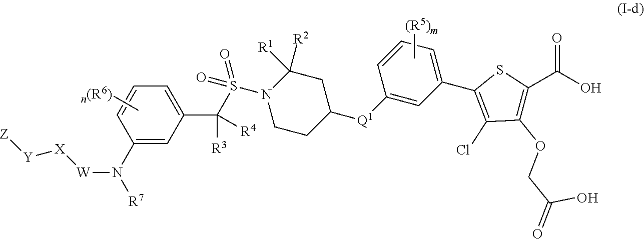

In some embodiments, m is 0. In some embodiments, m is 1; and R⁵ is —F.

In some embodiments, Q¹ is NH; or wherein Q¹ is —O—.

In some embodiments, R¹ and R² are both hydrogen. In some embodiments, R¹ and R² are independently C1-C3 alkyl. In some embodiments, R¹ and R² are both methyl.

In some embodiments, R³ and R⁴ are independently hydrogen, C1-C6 alkyl, or phenyl. In some embodiments, R³ and R⁴ are both hydrogen.

In some embodiments, n is 0. In some embodiments, n is 1; and R⁶ is —F.

In some embodiments, R¹ is hydrogen.

In some embodiments, W is —(CR^{A}R^{B})p¹-*, wherein the asterisk represents the point of attachment to X. In some embodiments, W is —NR^{C}(CR^{A}R^{B})p¹-* or —(CR^{A}R^{B})p¹-NR^{C}—*, wherein the asterisk represents the point of attachment to X.

In some embodiments, Y is —(CR^{A}R^{B})p²-*, wherein the asterisk represents the point of attachment to Z.

In some embodiments, Y is —(CR^{A}R^{B})p²-NR^{C}*, wherein the asterisk represents the point of attachment to Z.

In some embodiments, Y is —NR^{C}(CR^{A}R^{B})p²-*, wherein the asterisk represents the point of attachment to Z.

In some embodiments, Y is —(C═O)(CR^{A}R^{B})p²-NR^{C}—*, wherein the asterisk represents the point of attachment to Z.

In some embodiments, Y is —(CR^{A}R^{B})p⁴ (C═O)NR^{C}(CR^{A}R^{B})p³-*, wherein the asterisk represents the point of attachment to Z.

In some embodiments, X is a bond; and W is —((CR^{A}R^{B})pO)t-*. In some embodiments, —((CR^{A}R^{B})pO)t-* is —((CR^{A}R^{B})2O)t-*.

In some embodiments, R^{A} and R^{B} are independently hydrogen, fluoro, or C1-C3 alkyl. In some embodiments, R^{A} and R^{B} are both hydrogen.

In some embodiments, p¹ is 0, 1, 2, 3, 4, or 5. In some embodiments, p¹ is 0. In some embodiments, p¹ is 1. In some embodiments, p¹ is 2. In some embodiments, p¹ is 3. In some embodiments, p¹ is 4. In some embodiments, p¹ is 5.

In some embodiments, p² is 0, 1, 2, 3, 4, or 5. In some embodiments, p² is 0. In some embodiments, p² is 1. In some embodiments, p² is 2. In some embodiments, p² is 3. In some embodiments, p² is 4. In some embodiments, p² is 5.

In some embodiments, p³ is 0, 1, 2, 3, 4, or 5. In some embodiments, p³ is 0. In some embodiments, p³ is 1. In some embodiments, p² is 2. In some embodiments, p³ is 3. In some embodiments, p³ is 4. In some embodiments, p³ is 5.

In some embodiments, p¹ and p² are independently 0, 1, 2, 3, 4, or 5. In some embodiments, p² and p³ are independently 0, 1, 2, 3, 4, or 5.

In some embodiments, p¹+p²=0. In some embodiments, p¹+p²=1 or 2. In some embodiments, p¹+p²=1. In some embodiments, p¹+p²=2. In some embodiments, p¹+p²≤3. In some embodiments, p¹+p²=3. In some embodiments, p¹+p²=4. In some embodiments, p¹+p²=5. In some embodiments, p¹+p²=6. In some embodiments, p¹+p²=7. In some embodiments, p¹+p²=8. In some embodiments, p¹+p²=9. In some embodiments, p¹+p²=10. In some embodiments, p¹+p²=4, 5, 6, 7, 8, or 9.

In some embodiments, p²+p³=0. In some embodiments, p²+p³=1 or 2. In some embodiments, p²+p³=1. In some embodiments, p²+p³=2. In some embodiments, p²+p³≤3. In some embodiments, p²+p³=3. In some embodiments, p²+p³=4. In some embodiments, p²+p³=5. In some embodiments, p²+p³=6. In some embodiments, p²+p³=7. In some embodiments, p²+p³=8. In some embodiments, p²+p³=9. In some embodiments, p²+p³=10. In some embodiments, p²+p³=4, 5, 6, 7, 8, or 9. In some embodiments, t is 1, 2, or 3. In some embodiments, t is 1. In some embodiments, t is 2.

In some embodiments, t is 3.

In some embodiments, t+p²=3, 4, 5, or 6. In some embodiments, t+p²=3. In some embodiments, t+p²=4. In some embodiments, t+p²=5. In some embodiments, t+p²=6.

In some embodiments, t+p²+p³=3, 4, 5, or 6. In some embodiments, t+p²+p³=3. In some embodiments, t+p²+p³=4. In some embodiments, t+p²+p³=5. In some embodiments, t+p²+p³=6.

In some embodiments, X is 4-6 membered monocyclic heterocyclyl optionally substituted with 1-3 substituents independently selected from halogen atoms, C1-C6 alkyl, and hydroxyl. In some embodiments, X is selected from the group consisting of azetidinyl, piperidinyl, and piperazinyl. In some embodiments, X is

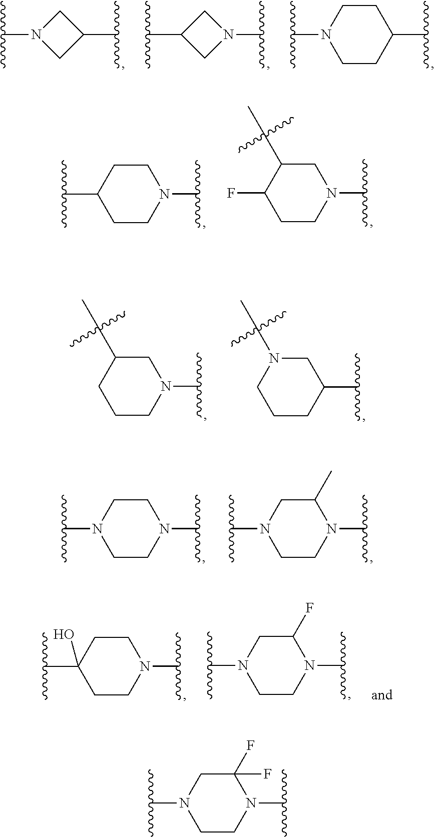

In some embodiments, X is

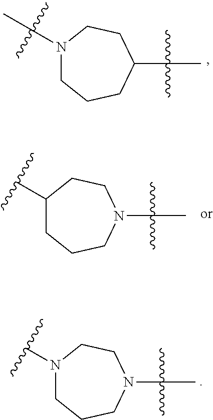 [SMILES: *C1CCCN(*)CC1 ; *C1CCCN(*)CC1 ; *N1CCCN(*)CC1]

In some embodiments, X is 6-10 membered bicyclic heterocyclyl. In some embodiments, X is 6-10 membered bicyclic fused heterocyclyl. In some embodiments, X is 7-10 membered bicyclic spiroheterocyclyl. In some embodiments, X is

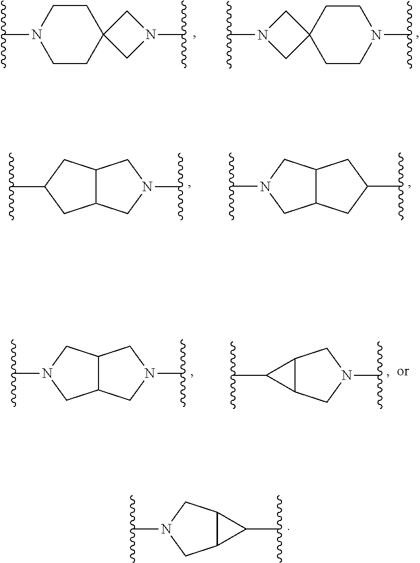

In some embodiments, X is triazolyl.

In some embodiments, X is pyrazolyl.

In some embodiments, W—X—Y is selected from the group consisting of:

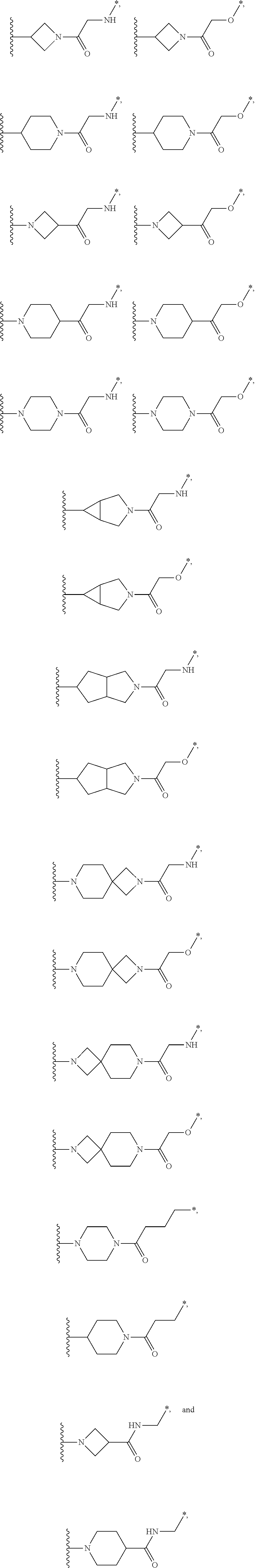

wherein the asterisk represents the point of attachment to Z.

In some embodiments, W—X—Y is selected from the group consisting of:

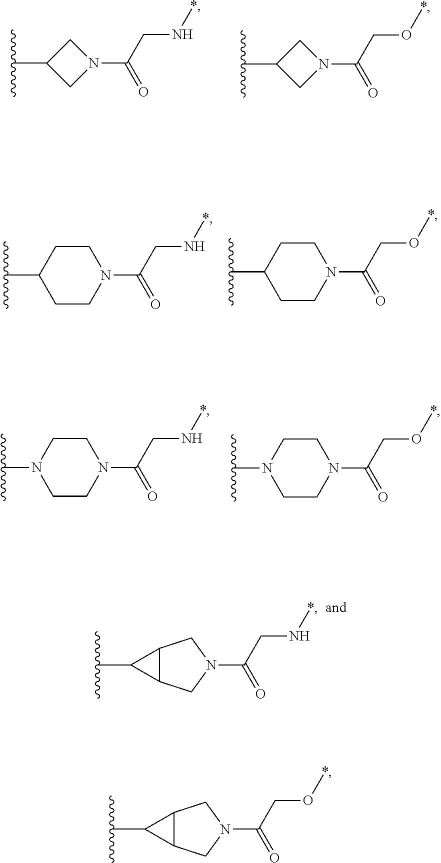

wherein the asterisk represents the point of attachment to Z.

In some embodiments, W—X—Y is selected from the group consisting of:

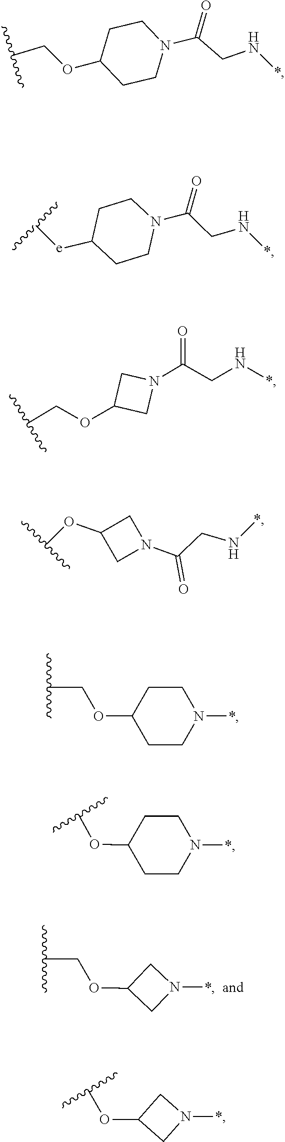

wherein the asterisk represents the point of attachment to Z.

In some embodiments, W—X—Y is selected from the group consisting of:

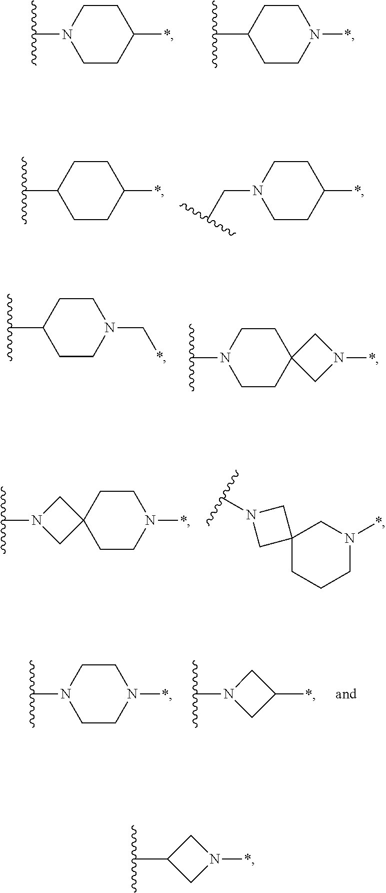

wherein the asterisk represents the point of attachment to Z.

In some embodiments, W—X—Y is selected from the group consisting of:

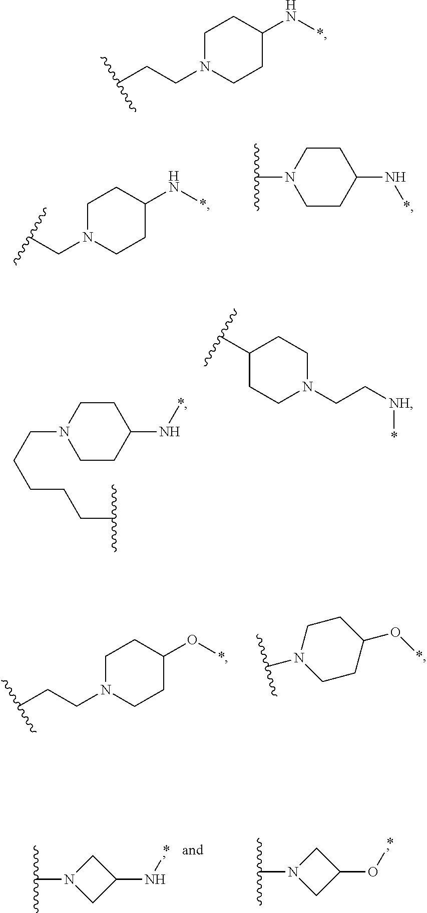

wherein the asterisk represents the point of attachment to Z.

In some embodiments, W—X—Y is selected from the group consisting of:

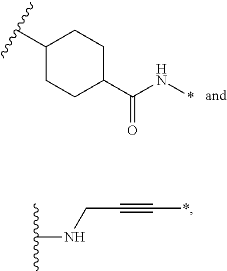

wherein the asterisk represents the point of attachment to Z.

In some embodiments, the asterisk represents the point of attachment to Z.

In some embodiments, W—X—Y is selected from the group consisting of:

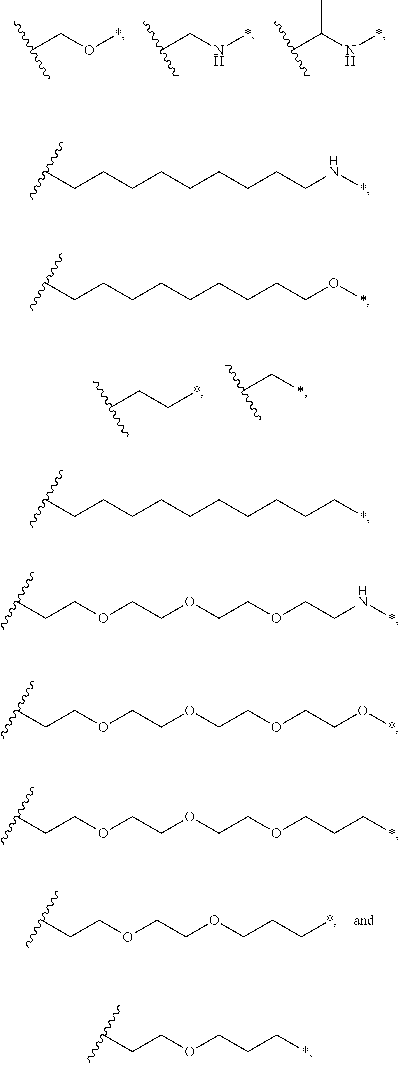

wherein the asterisk represents the point of attachment to Z.

In some embodiments, W—X—Y is selected from the group consisting of:

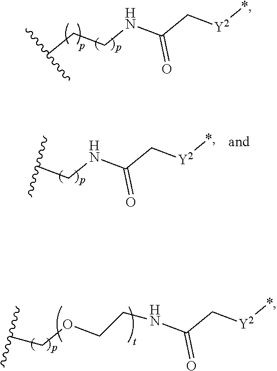

wherein Y² is NH, NMe, O, or CH₂; and the asterisk represents the point of attachment to Z. In some embodiments, Y² is NH. In some embodiments, Y² is NMe. In some embodiments, Y² is O. In some embodiments, Y² is CH₂. In some embodiments, t is 1, 2, or 3. In some embodiments, t is 1. In some embodiments, t is 2. In some embodiments, tis 3. In some embodiments, each p is independently 1, 2, or 3. In some embodiments, each p is 1. In some embodiments, each p is 2. In some embodiments, each p is 3.

In some embodiments, W—X—Y is selected from the group consisting of:

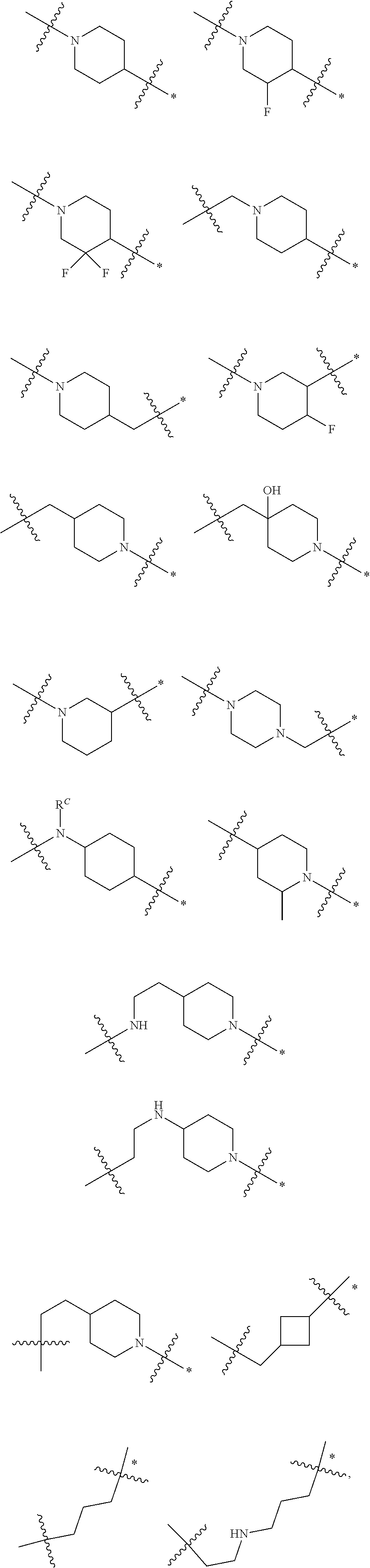

wherein the asterisk represents the point of attachment to Z.

In some embodiments, W—X—Y is selected from the group consisting of:

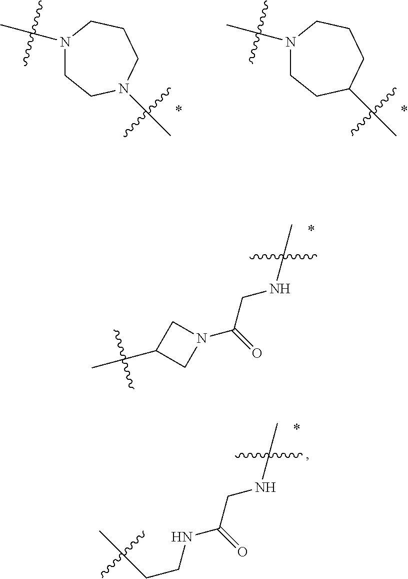 [SMILES: *N1CCCN(*)CC1 ; *C1CCCN(*)CC1 ; *NCC(=O)N1CC(*)C1 ; *CCNC(=O)CN*]

wherein the asterisk represents the point of attachment to Z.

In some embodiments, W—X—Y is selected from the group consisting of:

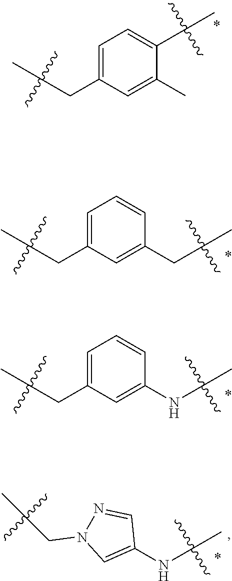

wherein the asterisk represents the point of attachment to Z.

In some embodiments, the shortest distance between Ring B and Z is from about 7 Å to about 25 Å, for example, about 7 Å, about &Å, about 9 Å, about 10 Å, about 11 Å, about 12 Å, about 13 Å, about 14 Å, about 15 Å, about 16 Å, about 17 Å, about 18 Å, about 19 Å, about 20 Å, about 21 Å, about 22 Å, about 23 Å, about 24 Å, about 25 Å, or any value in between. In some embodiments, the shortest distance between Ring B and Z is from about 7 Å to about 22.5 Å. In some embodiments, the shortest distance between Ring B and Z is from about 7 Å to about 20 Å. In some embodiments, the shortest distance between Ring B and Z is from about 7 Å to about 17.5 Å. In some embodiments, the shortest distance between Ring B and Z is from about 7 Å to about 15 Å. In some embodiments, the shortest distance between Ring B and Z is from about 7 Å to about 12.5 Å. In some embodiments, the shortest distance between Ring B and Z is from about 7 Å to about 10 Å.

In some embodiments, the shortest path, by number of bonds, between the atom of Ring B connected to the NR⁷ group and the atom serving as the point of attachment in the Z group is about 7 bonds to about 18 bonds, for example, about 7 bonds, about 8 bonds, about 9 bonds, about 10 bonds, about 11 bonds, about 12 bonds, about 13 bonds, about 14 bonds, about 15 bonds, about 16 bonds, about 17 bonds, about 18 bonds, or any value in between.

In some embodiments, the shortest path, by number of bonds, between the atom of Ring B connected to the NR⁷ group and the atom serving as the point of attachment in the Z group is about 7 bonds to about 16 bonds. In some embodiments, the shortest path, by number of bonds, between the atom of Ring B connected to the NR⁷ group and the atom serving as the point of attachment in the Z group is about 7 bonds to about 14 bonds. In some embodiments, the shortest path, by number of bonds, between the atom of Ring B connected to the NR⁷ group and the atom serving as the point of attachment in the Z group is about 7 bonds to about 12 bonds. In some embodiments, the shortest path, by number of bonds, between the atom of Ring B connected to the NR⁷ group and the atom serving as the point of attachment in the Z group is 7 bonds, 8 bonds, or 9 bonds. In some embodiments, the shortest path, by number of bonds, between the atom of Ring B connected to the NR⁷ group and the atom serving as the point of attachment in the Z group is about 8 bonds to about 16 bonds. In some embodiments, the shortest path, by number of bonds, between the atom of Ring B connected to the NR⁷ group and the atom serving as the point of attachment in the Z group is about 8 bonds to about 14 bonds. In some embodiments, the shortest path, by number of bonds, between the atom of Ring B connected to the NR⁷ group and the atom serving as the point of attachment in the Z group is about 8 bonds to about 12 bonds. In some embodiments, the shortest path, by number of bonds, between the atom of Ring B connected to the NR⁷ group and the atom serving as the point of attachment in the Z group is 8 bonds or 9 bonds. In some embodiments, the shortest path, by number of bonds, between the atom of Ring B connected to the NR⁷ group and the atom serving as the point of attachment in the Z group is 10 bonds, 11 bonds, 12 bonds, or 13 bonds.

In some embodiments, one of J, U, V, W, and X is a bond. In some embodiments, two of J, U, V, W, and X is a bond. In some embodiments, three of J, U, V, W, and X is a bond. In some embodiments, J, U, V, W, and X cannot each be a bond.

In some embodiments, the compound of Formula (I) is selected from the compounds described in Table 1, or a pharmaceutically acceptable salt thereof.

TABLE 1

| Com- | |
|---|---|
| pound | |
| No. | Structure |
| 1 | 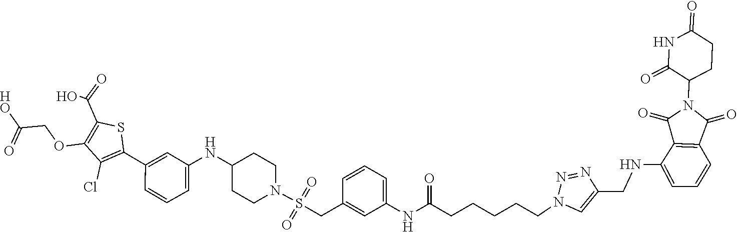 [SMILES: O=C(O)COc1c(C(=O)O)sc(-c2cccc(NC3CCN(S(=O)(=O)Cc4cccc(NC(=O)CCCCCn5cc(CNc6cccc7c6C(=O)N(C6CCC(=O)NC6=O)C7=O)nn5)c4)CC3)c2)c1Cl] |
| 2 | 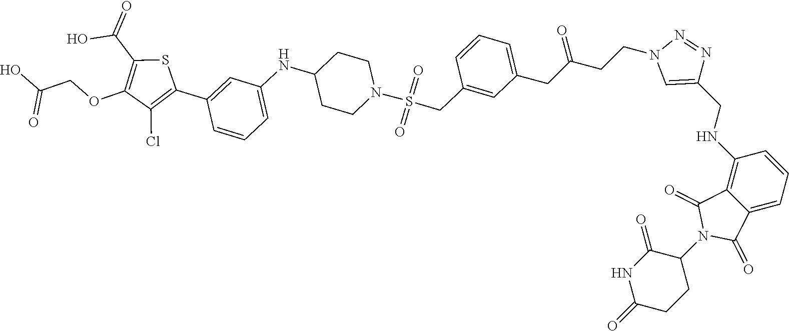 [SMILES: O=C(O)COc1c(C(=O)O)sc(-c2cccc(NC3CCN(S(=O)(=O)Cc4cccc(CC(=O)CCn5cc(CNc6cccc7c6C(=O)N(C6CCC(=O)NC6=O)C7=O)nn5)c4)CC3)c2)c1Cl] |
| 3 | 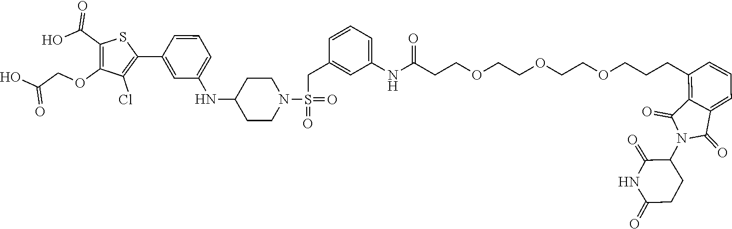 [SMILES: O=C(O)COc1c(C(=O)O)sc(-c2cccc(NC3CCN(S(=O)(=O)Cc4cccc(NC(=O)CCOCCOCCOCCCc5cccc6c5C(=O)N(C5CCC(=O)NC5=O)C6=O)c4)CC3)c2)c1Cl] |
| 4 | 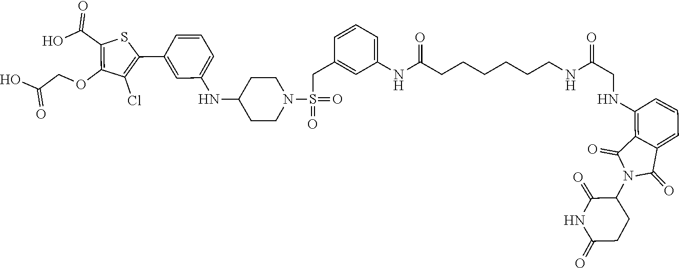 [SMILES: O=C(O)COc1c(C(=O)O)sc(-c2cccc(NC3CCN(S(=O)(=O)Cc4cccc(NC(=O)CCCCCCNC(=O)CNc5cccc6c5C(=O)N(C5CCC(=O)NC5=O)C6=O)c4)CC3)c2)c1Cl] |
| 5 | 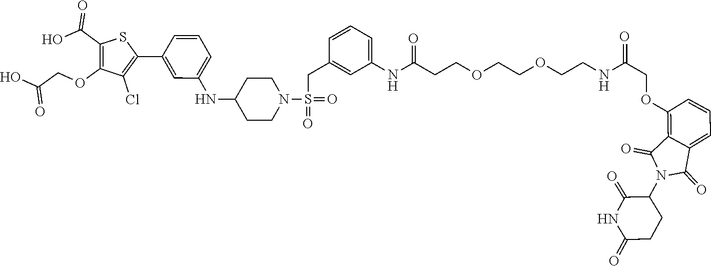 [SMILES: O=C(O)COc1c(C(=O)O)sc(-c2cccc(NC3CCN(S(=O)(=O)Cc4cccc(NC(=O)CCOCCOCCNC(=O)COc5cccc6c5C(=O)N(C5CCC(=O)NC5=O)C6=O)c4)CC3)c2)c1Cl] |
| 6 | 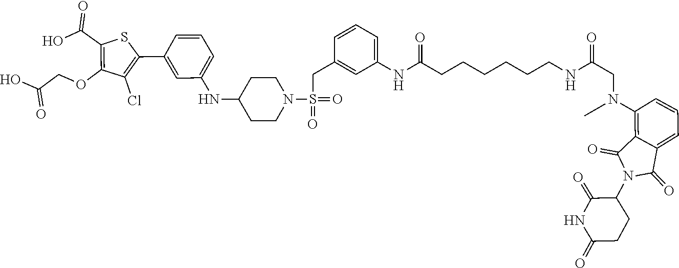 [SMILES: CN(CC(=O)NCCCCCCC(=O)Nc1cccc(CS(=O)(=O)N2CCC(Nc3cccc(-c4sc(C(=O)O)c(OCC(=O)O)c4Cl)c3)CC2)c1)c1cccc2c1C(=O)N(C1CCC(=O)NC1=O)C2=O] |
| 7 | 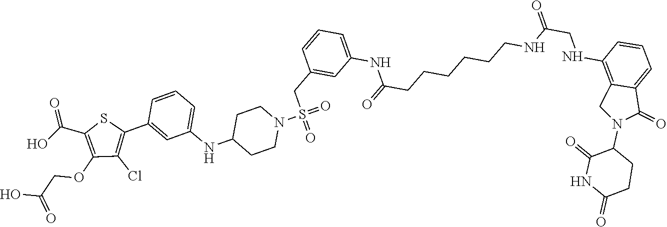 [SMILES: O=C(O)COc1c(C(=O)O)sc(-c2cccc(NC3CCN(S(=O)(=O)Cc4cccc(NC(=O)CCCCCCNC(=O)CNc5cccc6c5CN(C5CCC(=O)NC5=O)C6=O)c4)CC3)c2)c1Cl] |
| 8 | 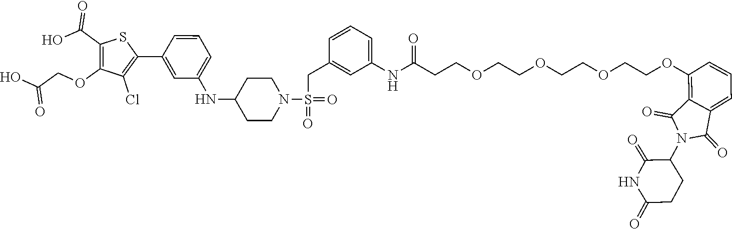 [SMILES: O=C(O)COc1c(C(=O)O)sc(-c2cccc(NC3CCN(S(=O)(=O)Cc4cccc(NC(=O)CCOCCOCCOCCOc5cccc6c5C(=O)N(C5CCC(=O)NC5=O)C6=O)c4)CC3)c2)c1Cl] |
| 9 | 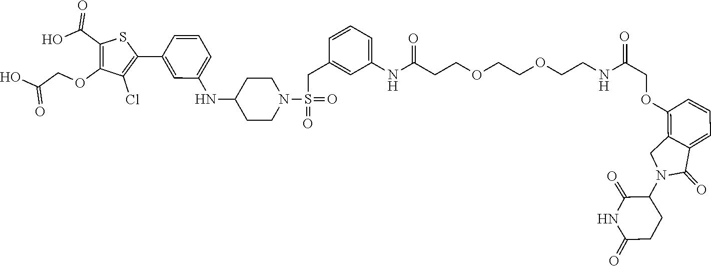 [SMILES: O=C(O)COc1c(C(=O)O)sc(-c2cccc(NC3CCN(S(=O)(=O)Cc4cccc(NC(=O)CCOCCOCCNC(=O)COc5cccc6c5CN(C5CCC(=O)NC5=O)C6=O)c4)CC3)c2)c1Cl] |
| 10 | 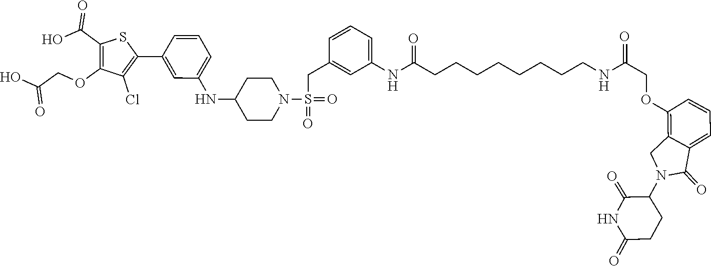 [SMILES: O=C(O)COc1c(C(=O)O)sc(-c2cccc(NC3CCN(S(=O)(=O)Cc4cccc(NC(=O)CCCCCCCCNC(=O)COc5cccc6c5CN(C5CCC(=O)NC5=O)C6=O)c4)CC3)c2)c1Cl] |
| 11 | 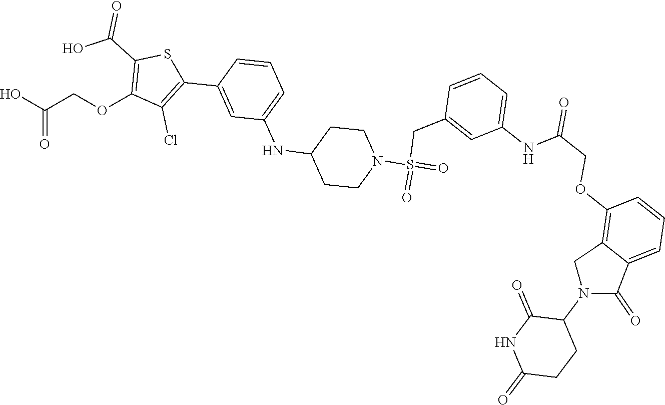 [SMILES: O=C(O)COc1c(C(=O)O)sc(-c2cccc(NC3CCN(S(=O)(=O)Cc4cccc(NC(=O)COc5cccc6c5CN(C5CCC(=O)NC5=O)C6=O)c4)CC3)c2)c1Cl] |
| 12 | 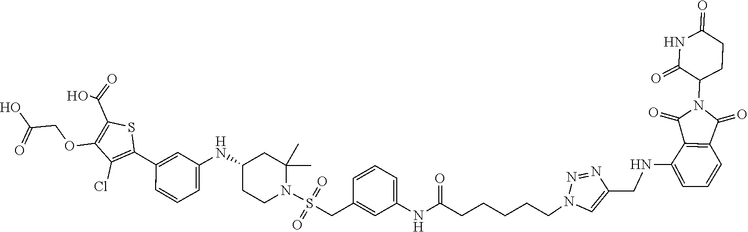 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCCCCn2cc(CNc3cccc4c3C(=O)N(C3CCC(=O)NC3=O)C4=O)nn2)c1] |
| 13 | 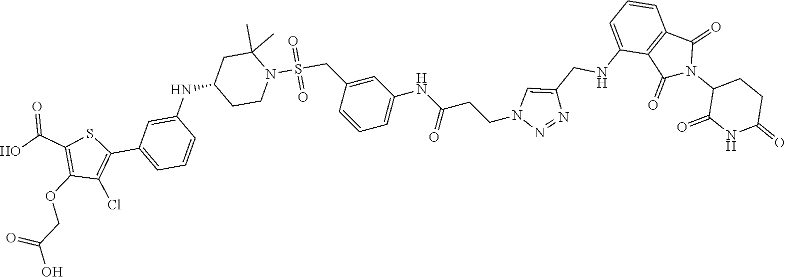 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCn2cc(CNc3cccc4c3C(=O)N(C3CCC(=O)NC3=O)C4=O)nn2)c1] |
| 14 | 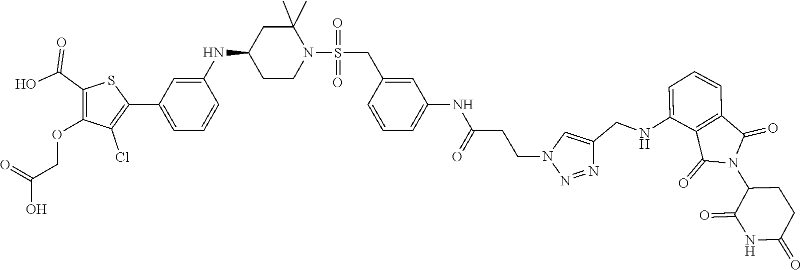 [SMILES: CC1(C)C[C@H](Nc2cccc(-c3sc(C(=O)O)c(OCC(=O)O)c3Cl)c2)CCN1S(=O)(=O)Cc1cccc(NC(=O)CCn2cc(CNc3cccc4c3C(=O)N(C3CCC(=O)NC3=O)C4=O)nn2)c1] |
| 15 | 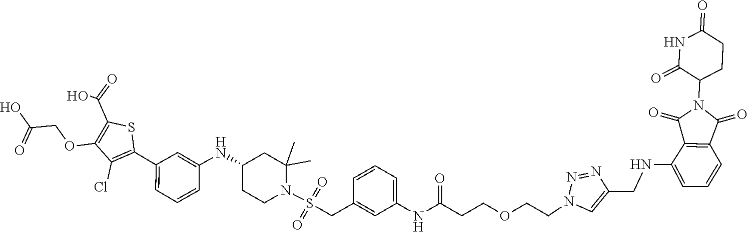 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCOCCn2cc(CNc3cccc4c3C(=O)N(C3CCC(=O)NC3=O)C4=O)nn2)c1] |
| 16 | 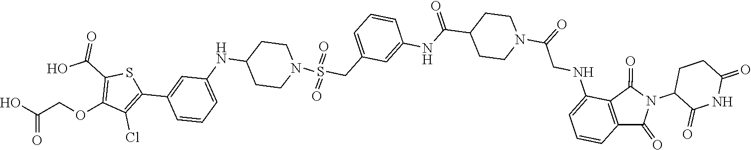 [SMILES: O=C(O)COc1c(C(=O)O)sc(-c2cccc(NC3CCN(S(=O)(=O)Cc4cccc(NC(=O)C5CCN(C(=O)CNc6cccc7c6C(=O)N(C6CCC(=O)NC6=O)C7=O)CC5)c4)CC3)c2)c1Cl] |
| 17 | 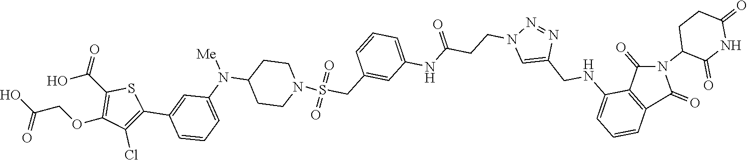 [SMILES: CN(c1cccc(-c2sc(C(=O)O)c(OCC(=O)O)c2Cl)c1)C1CCN(S(=O)(=O)Cc2cccc(NC(=O)CCn3cc(CNc4cccc5c4C(=O)N(C4CCC(=O)NC4=O)C5=O)nn3)c2)CC1] |
| 18 | 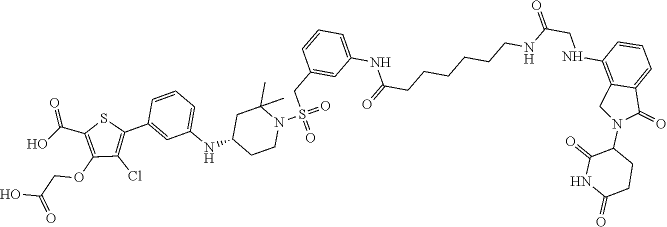 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCCCCCNC(=O)CNc2cccc3c2CN(C2CCC(=O)NC2=O)C3=O)c1] |
| 19 | 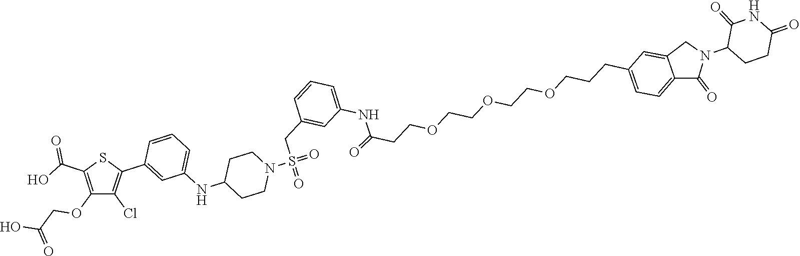 [SMILES: O=C(O)COc1c(C(=O)O)sc(-c2cccc(NC3CCN(S(=O)(=O)Cc4cccc(NC(=O)CCOCCOCCOCCCc5ccc6c(c5)CN(C5CCC(=O)NC5=O)C6=O)c4)CC3)c2)c1Cl] |
| 20 | 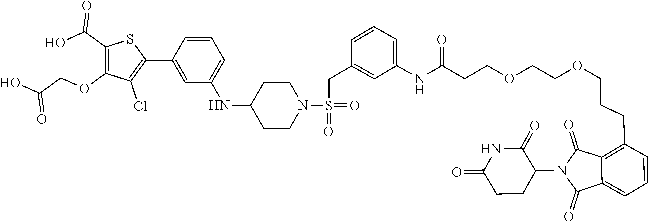 [SMILES: O=C(O)COc1c(C(=O)O)sc(-c2cccc(NC3CCN(S(=O)(=O)Cc4cccc(NC(=O)CCOCCOCCCc5cccc6c5C(=O)N(C5CCC(=O)NC5=O)C6=O)c4)CC3)c2)c1Cl] |
| 21 | 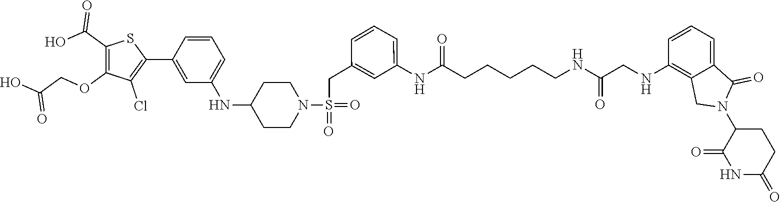 [SMILES: O=C(O)COc1c(C(=O)O)sc(-c2cccc(NC3CCN(S(=O)(=O)Cc4cccc(NC(=O)CCCCCNC(=O)CNc5cccc6c5CN(C5CCC(=O)NC5=O)C6=O)c4)CC3)c2)c1Cl] |
| 22 | 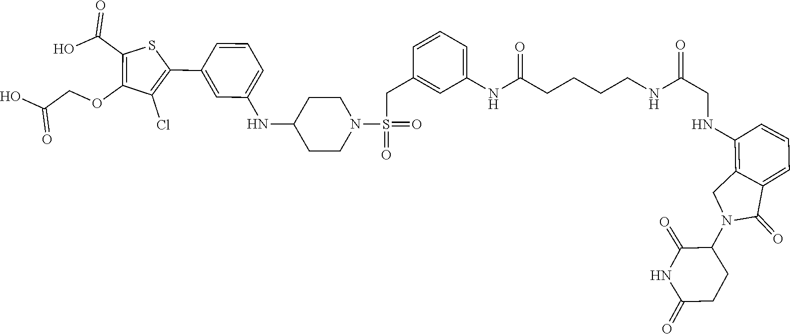 [SMILES: O=C(O)COc1c(C(=O)O)sc(-c2cccc(NC3CCN(S(=O)(=O)Cc4cccc(NC(=O)CCCCNC(=O)CNc5cccc6c5CN(C5CCC(=O)NC5=O)C6=O)c4)CC3)c2)c1Cl] |
| 23 | 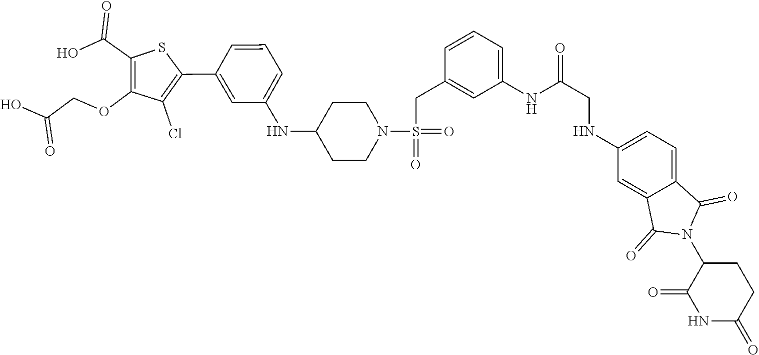 [SMILES: O=C(O)COc1c(C(=O)O)sc(-c2cccc(NC3CCN(S(=O)(=O)Cc4cccc(NC(=O)CNc5ccc6c(c5)C(=O)N(C5CCC(=O)NC5=O)C6=O)c4)CC3)c2)c1Cl] |
| 24 | 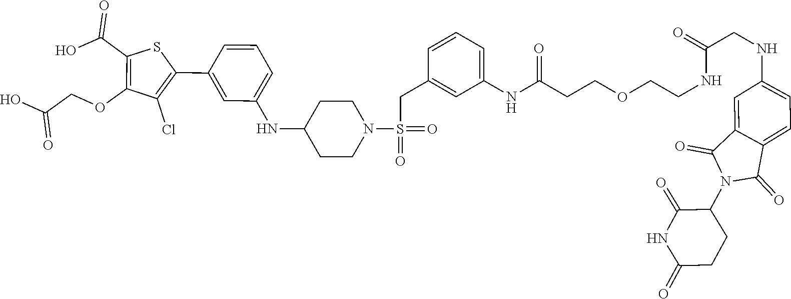 [SMILES: O=C(O)COc1c(C(=O)O)sc(-c2cccc(NC3CCN(S(=O)(=O)Cc4cccc(NC(=O)CCOCCNC(=O)CNc5ccc6c(c5)C(=O)N(C5CCC(=O)NC5=O)C6=O)c4)CC3)c2)c1Cl] |
| 25 | 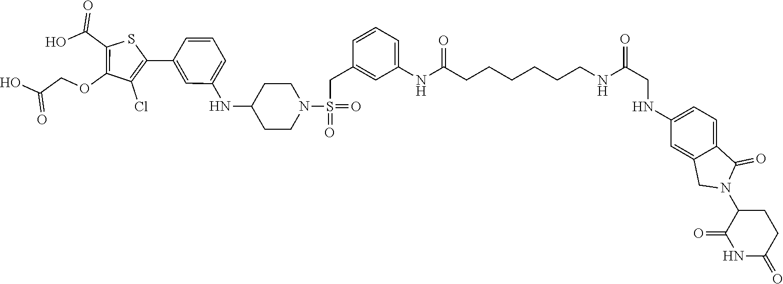 [SMILES: O=C(O)COc1c(C(=O)O)sc(-c2cccc(NC3CCN(S(=O)(=O)Cc4cccc(NC(=O)CCCCCCNC(=O)CNc5ccc6c(c5)CN(C5CCC(=O)NC5=O)C6=O)c4)CC3)c2)c1Cl] |
| 26 | 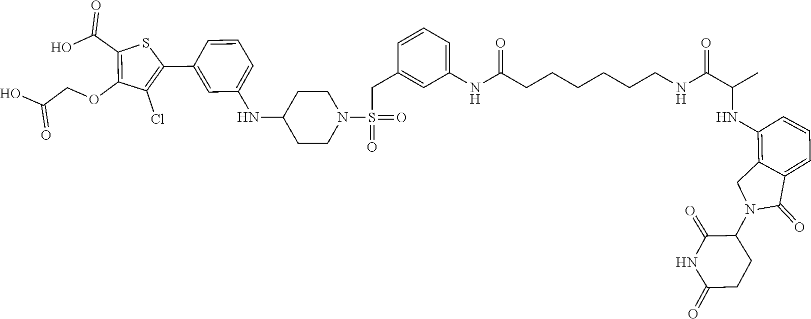 [SMILES: CC(Nc1cccc2c1CN(C1CCC(=O)NC1=O)C2=O)C(=O)NCCCCCCC(=O)Nc1cccc(CS(=O)(=O)N2CCC(Nc3cccc(-c4sc(C(=O)O)c(OCC(=O)O)c4Cl)c3)CC2)c1] |
| 27 | 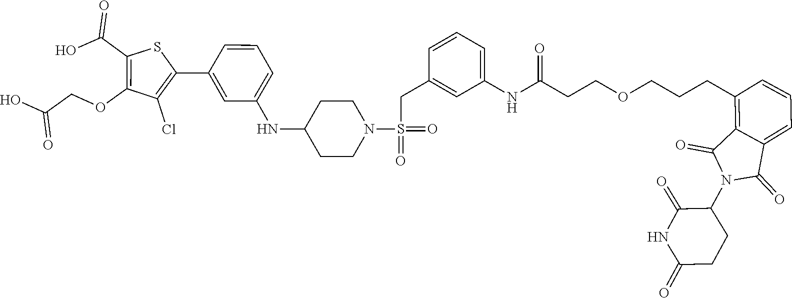 [SMILES: O=C(O)COc1c(C(=O)O)sc(-c2cccc(NC3CCN(S(=O)(=O)Cc4cccc(NC(=O)CCOCCCc5cccc6c5C(=O)N(C5CCC(=O)NC5=O)C6=O)c4)CC3)c2)c1Cl] |
| 28 | 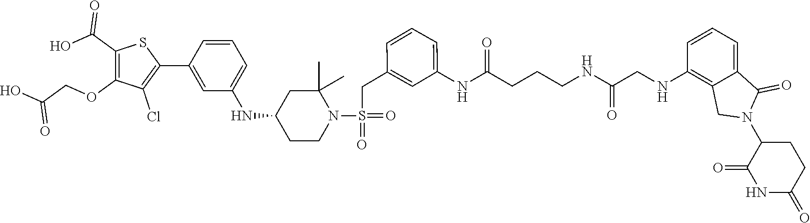 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCCNC(=O)CNc2cccc3c2CN(C2CCC(=O)NC2=O)C3=O)c1] |
| 29 | 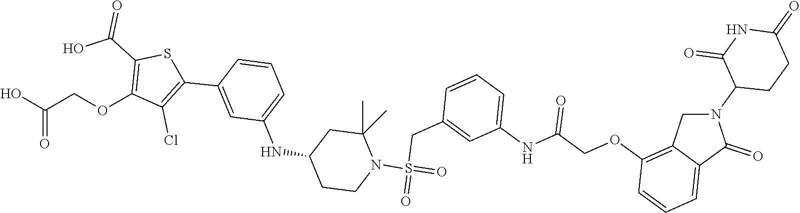 [SMILES: CC1(C)C[C@@H](Nc2cccc(-c3sc(C(=O)O)c(OCC(=O)O)c3Cl)c2)CCN1S(=O)(=O)Cc1cccc(NC(=O)COc2cccc3c2CN(C2CCC(=O)NC2=O)C3=O)c1] |
| 30 | 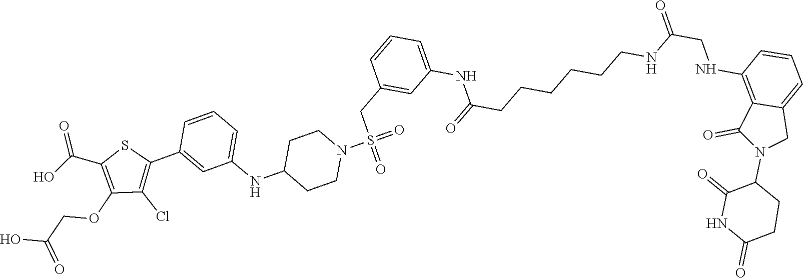 [SMILES: O=C(O)COc1c(C(=O)O)sc(-c2cccc(NC3CCN(S(=O)(=O)Cc4cccc(NC(=O)CCCCCCNC(=O)CNc5cccc6c5C(=O)N(C5CCC(=O)NC5=O)C6)c4)CC3)c2)c1Cl] |
| 31 | 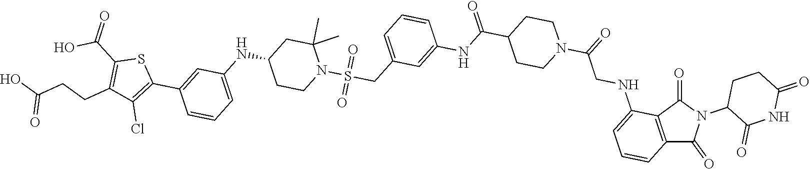 [SMILES: CC1(C)C[C@@H](Nc2cccc(-c3sc(C(=O)O)c(CCC(=O)O)c3Cl)c2)CCN1S(=O)(=O)Cc1cccc(NC(=O)C2CCN(C(=O)CNc3cccc4c3C(=O)N(C3CCC(=O)NC3=O)C4=O)CC2)c1] |
| 32 | 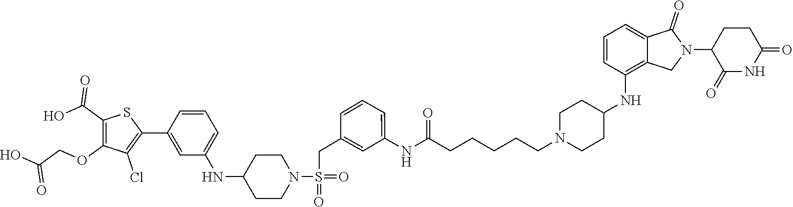 [SMILES: O=C(O)COc1c(C(=O)O)sc(-c2cccc(NC3CCN(S(=O)(=O)Cc4cccc(NC(=O)CCCCCN5CCC(Nc6cccc7c6CN(C6CCC(=O)NC6=O)C7=O)CC5)c4)CC3)c2)c1Cl] |
| 33 | 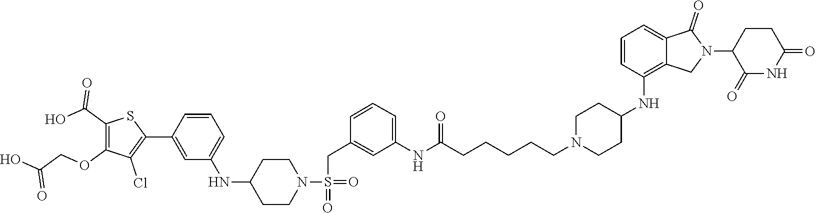 [SMILES: O=C(O)COc1c(C(=O)O)sc(-c2cccc(NC3CCN(S(=O)(=O)Cc4cccc(NC(=O)CCCCCN5CCC(Nc6cccc7c6CN(C6CCC(=O)NC6=O)C7=O)CC5)c4)CC3)c2)c1Cl] |
| 34 | 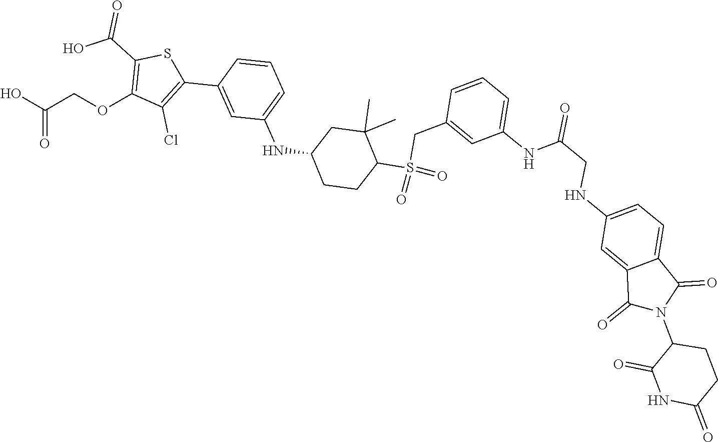 [SMILES: CC1(C)C[C@@H](Nc2cccc(-c3sc(C(=O)O)c(OCC(=O)O)c3Cl)c2)CCC1S(=O)(=O)Cc1cccc(NC(=O)CNc2ccc3c(c2)C(=O)N(C2CCC(=O)NC2=O)C3=O)c1] |
| 35 | 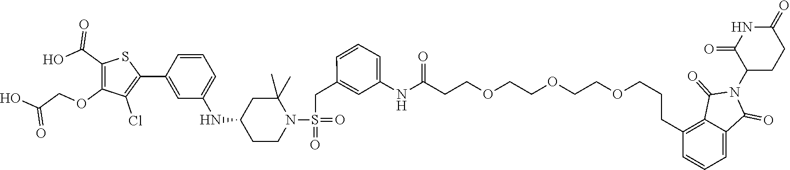 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCOCCOCCOCCCc2cccc3c2C(=O)N(C2CCC(=O)NC2=O)C3=O)c1] |
| 36 | 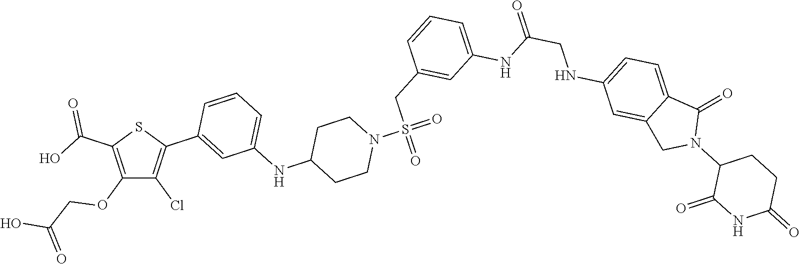 [SMILES: O=C(O)COc1c(C(=O)O)sc(-c2cccc(NC3CCN(S(=O)(=O)Cc4cccc(NC(=O)CNc5ccc6c(c5)CN(C5CCC(=O)NC5=O)C6=O)c4)CC3)c2)c1Cl] |
| 37 | 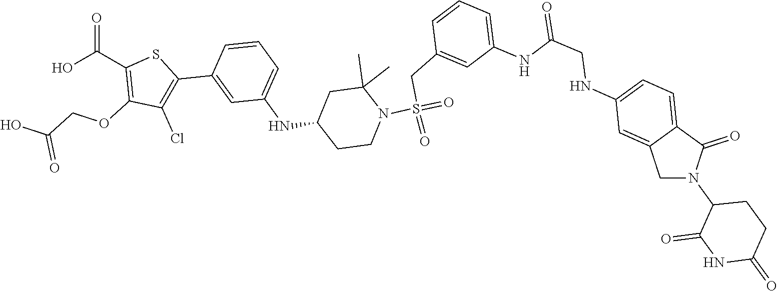 [SMILES: CC1(C)C[C@@H](Nc2cccc(-c3sc(C(=O)O)c(OCC(=O)O)c3Cl)c2)CCN1S(=O)(=O)Cc1cccc(NC(=O)CNc2ccc3c(c2)CN(C2CCC(=O)NC2=O)C3=O)c1] |
| 38 | 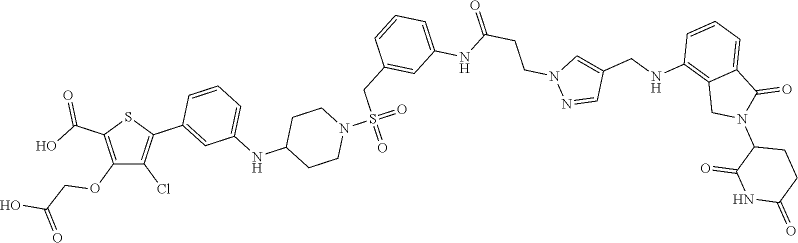 [SMILES: O=C(O)COc1c(C(=O)O)sc(-c2cccc(NC3CCN(S(=O)(=O)Cc4cccc(NC(=O)CCn5cc(CNc6cccc7c6CN(C6CCC(=O)NC6=O)C7=O)cn5)c4)CC3)c2)c1Cl] |
| 39 | 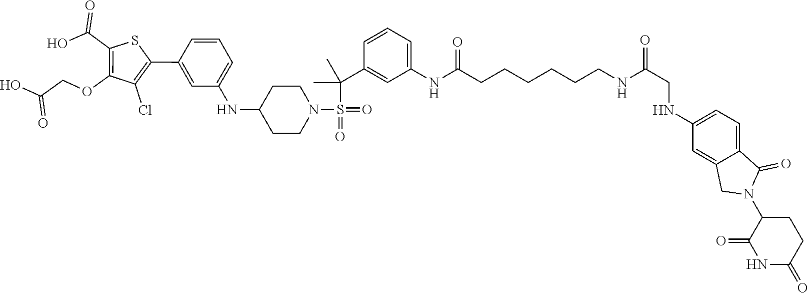 [SMILES: CC(C)(c1cccc(NC(=O)CCCCCCNC(=O)CNc2ccc3c(c2)CN(C2CCC(=O)NC2=O)C3=O)c1)S(=O)(=O)N1CCC(Nc2cccc(-c3sc(C(=O)O)c(OCC(=O)O)c3Cl)c2)CC1] |
| 40 | 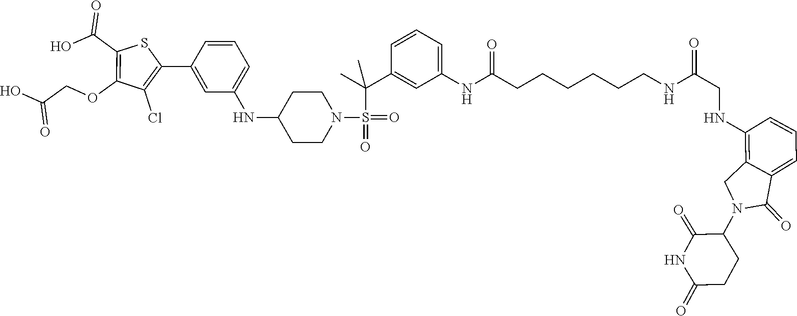 [SMILES: CC(C)(c1cccc(NC(=O)CCCCCCNC(=O)CNc2cccc3c2CN(C2CCC(=O)NC2=O)C3=O)c1)S(=O)(=O)N1CCC(Nc2cccc(-c3sc(C(=O)O)c(OCC(=O)O)c3Cl)c2)CC1] |
| 41 | 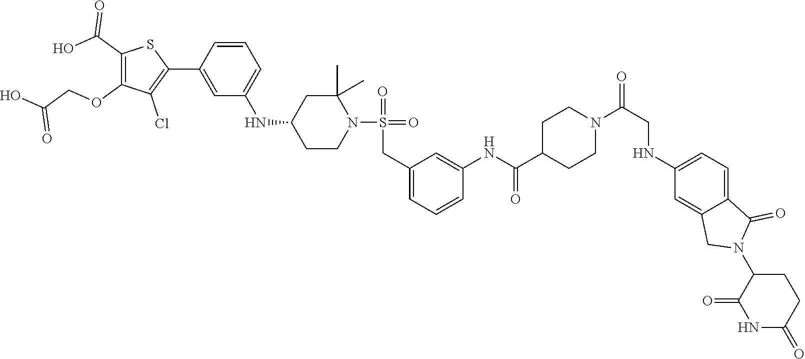 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3ccc4c(c3)CN(C3CCC(=O)NC3=O)C4=O)CC2)c1] |
| 42 | 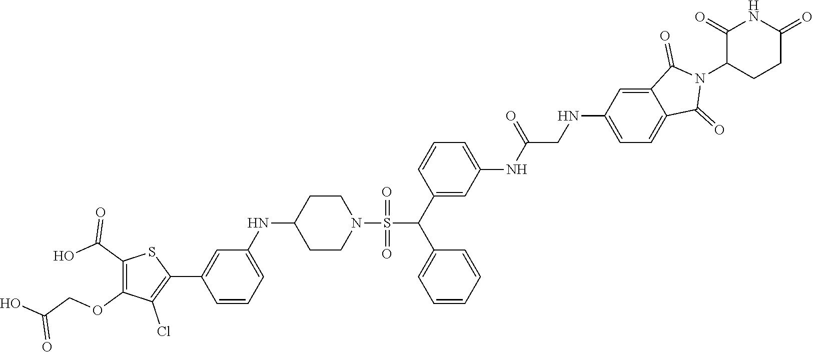 [SMILES: O=C(O)COc1c(C(=O)O)sc(-c2cccc(NC3CCN(S(=O)(=O)C(c4ccccc4)c4cccc(NC(=O)CNc5ccc6c(c5)C(=O)N(C5CCC(=O)NC5=O)C6=O)c4)CC3)c2)c1Cl] |
| 43 | 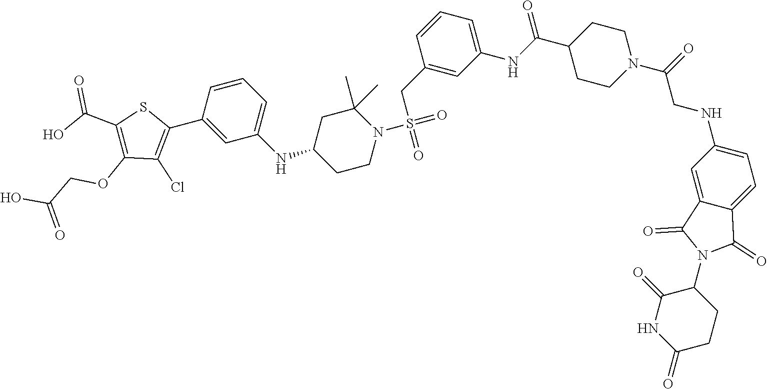 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3ccc4c(c3)C(=O)N(C3CCC(=O)NC3=O)C4=O)CC2)c1] |
| 44 | 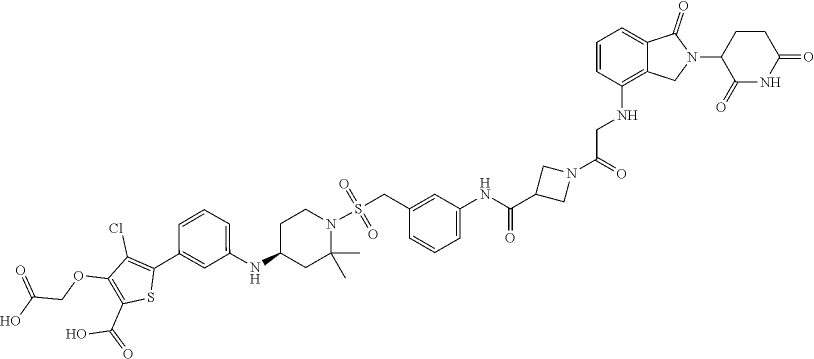 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3cccc4c3CN(C3CCC(=O)NC3=O)C4=O)C2)c1] |
| 44a | 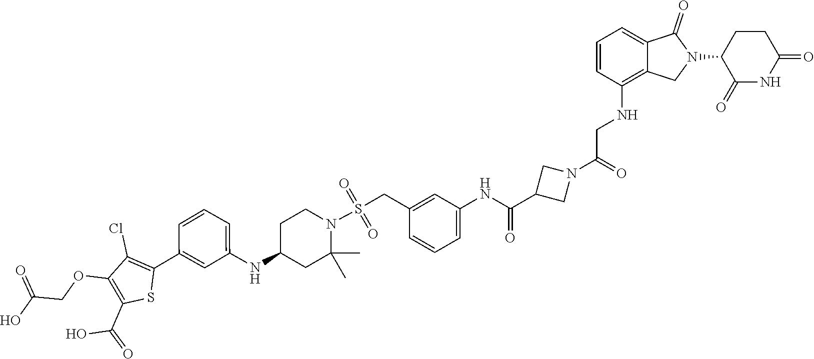 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3cccc4c3CN([C@@H]3CCC(=O)NC3=O)C4=O)C2)c1] |
| 44b | 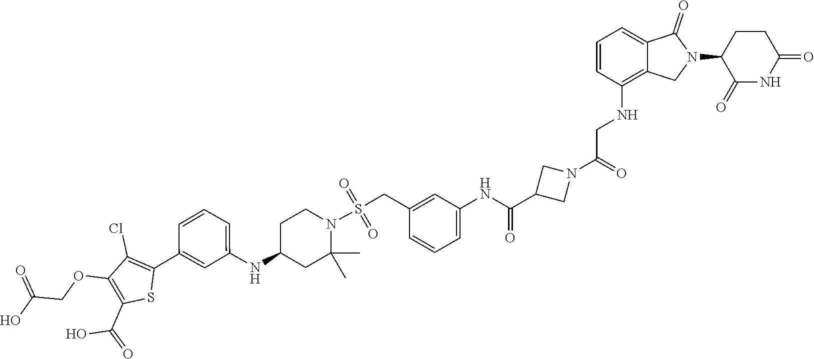 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3cccc4c3CN([C@H]3CCC(=O)NC3=O)C4=O)C2)c1] |
| 45 | 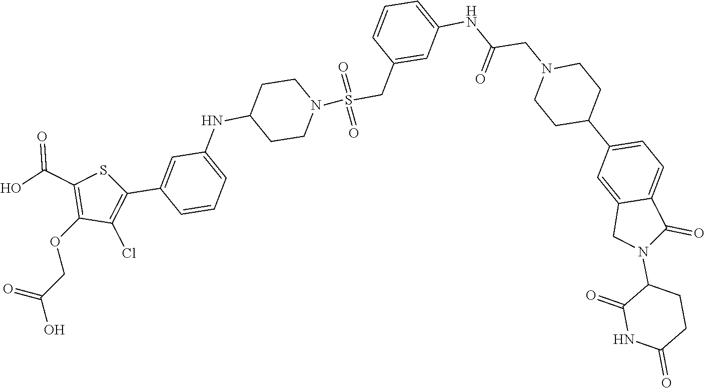 [SMILES: O=C(O)COc1c(C(=O)O)sc(-c2cccc(NC3CCN(S(=O)(=O)Cc4cccc(NC(=O)CN5CCC(c6ccc7c(c6)CN(C6CCC(=O)NC6=O)C7=O)CC5)c4)CC3)c2)c1Cl] |
| 46 | 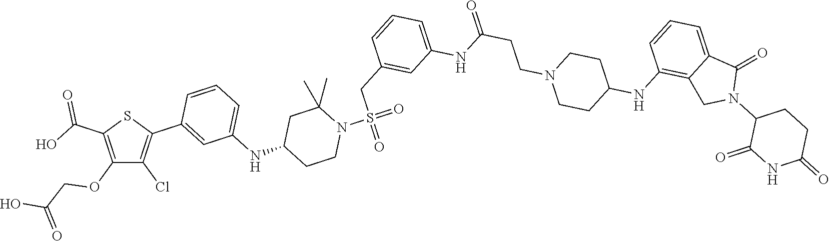 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCN2CCC(Nc3cccc4c3CN(C3CCC(=O)NC3=O)C4=O)CC2)c1] |
| 47 | 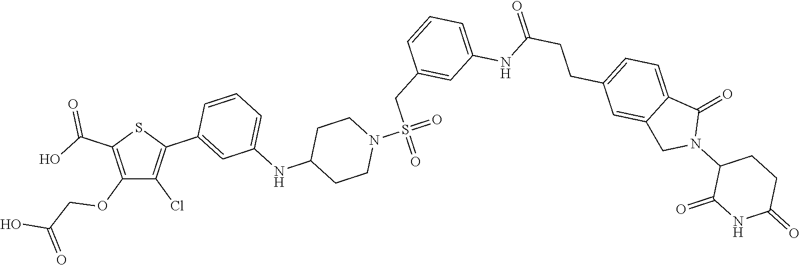 [SMILES: O=C(O)COc1c(C(=O)O)sc(-c2cccc(NC3CCN(S(=O)(=O)Cc4cccc(NC(=O)CCc5ccc6c(c5)CN(C5CCC(=O)NC5=O)C6=O)c4)CC3)c2)c1Cl] |
| 48 | 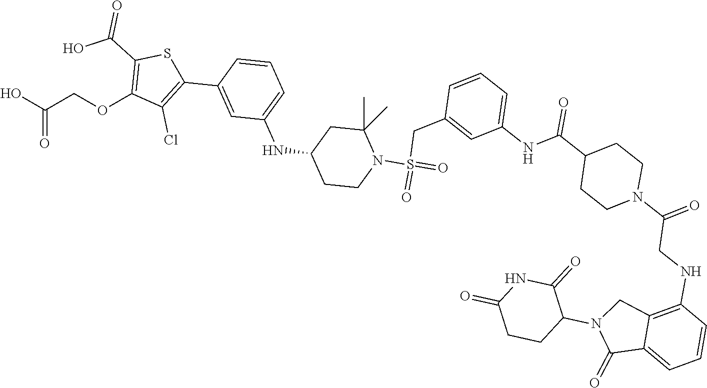 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3cccc4c3CN(C3CCC(=O)NC3=O)C4=O)CC2)c1] |
| 49 | 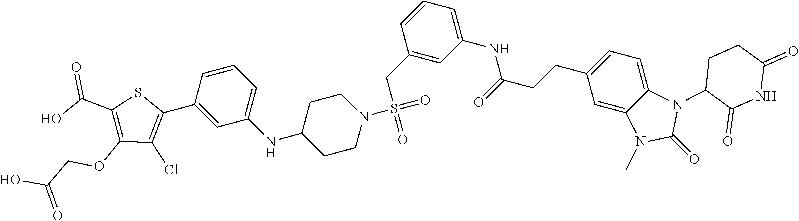 [SMILES: Cn1c(=O)n(C2CCC(=O)NC2=O)c2ccc(CCC(=O)Nc3cccc(CS(=O)(=O)N4CCC(Nc5cccc(-c6sc(C(=O)O)c(OCC(=O)O)c6Cl)c5)CC4)c3)cc21] |
| 50 | 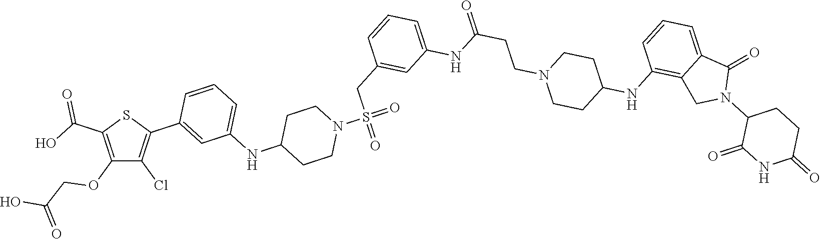 [SMILES: O=C(O)COc1c(C(=O)O)sc(-c2cccc(NC3CCN(S(=O)(=O)Cc4cccc(NC(=O)CCN5CCC(Nc6cccc7c6CN(C6CCC(=O)NC6=O)C7=O)CC5)c4)CC3)c2)c1Cl] |
| 51 | 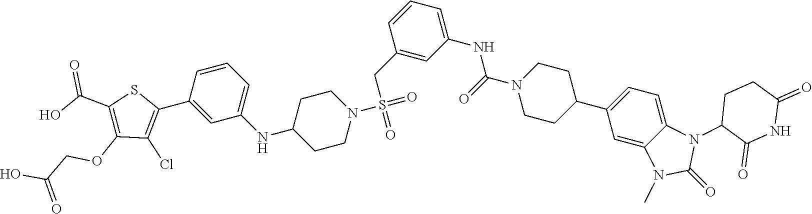 [SMILES: Cn1c(=O)n(C2CCC(=O)NC2=O)c2ccc(C3CCN(C(=O)Nc4cccc(CS(=O)(=O)N5CCC(Nc6cccc(-c7sc(C(=O)O)c(OCC(=O)O)c7Cl)c6)CC5)c4)CC3)cc21] |
| 52 | 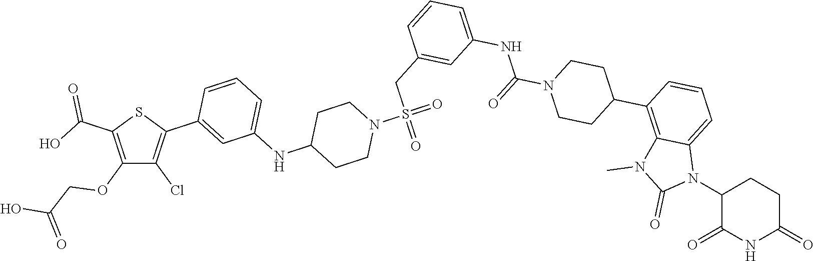 [SMILES: Cn1c(=O)n(C2CCC(=O)NC2=O)c2cccc(C3CCN(C(=O)Nc4cccc(CS(=O)(=O)N5CCC(Nc6cccc(-c7sc(C(=O)O)c(OCC(=O)O)c7Cl)c6)CC5)c4)CC3)c21] |
| 53 | 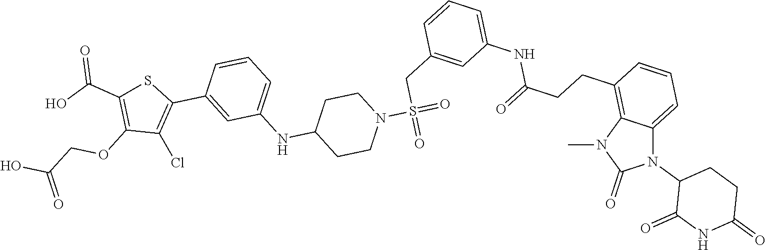 [SMILES: Cn1c(=O)n(C2CCC(=O)NC2=O)c2cccc(CCC(=O)Nc3cccc(CS(=O)(=O)N4CCC(Nc5cccc(-c6sc(C(=O)O)c(OCC(=O)O)c6Cl)c5)CC4)c3)c21] |
| 54 | 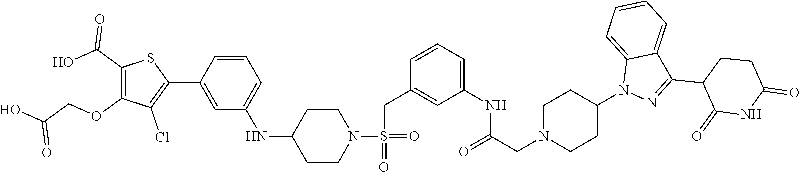 [SMILES: O=C(O)COc1c(C(=O)O)sc(-c2cccc(NC3CCN(S(=O)(=O)Cc4cccc(NC(=O)CN5CCC(n6nc(C7CCC(=O)NC7=O)c7ccccc76)CC5)c4)CC3)c2)c1Cl] |
| 55 | 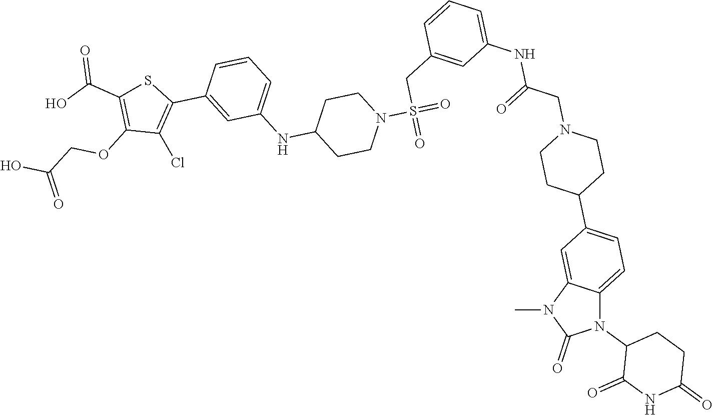 [SMILES: Cn1c(=O)n(C2CCC(=O)NC2=O)c2ccc(C3CCN(CC(=O)Nc4cccc(CS(=O)(=O)N5CCC(Nc6cccc(-c7sc(C(=O)O)c(OCC(=O)O)c7Cl)c6)CC5)c4)CC3)cc21] |
| 56 | 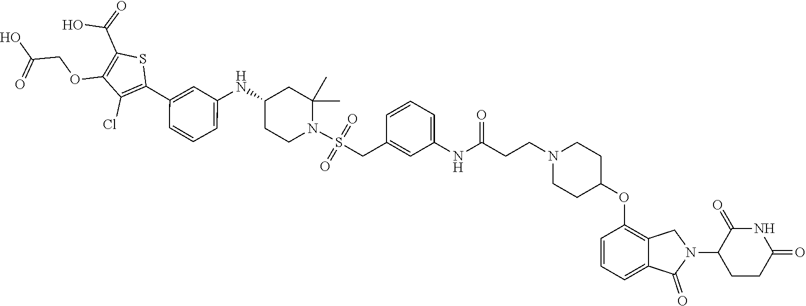 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCN2CCC(Oc3cccc4c3CN(C3CCC(=O)NC3=O)C4=O)CC2)c1] |
| 57 | 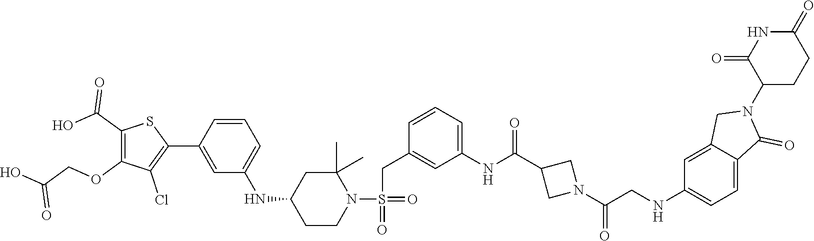 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3ccc4c(c3)CN(C3CCC(=O)NC3=O)C4=O)C2)c1] |
| 58 | 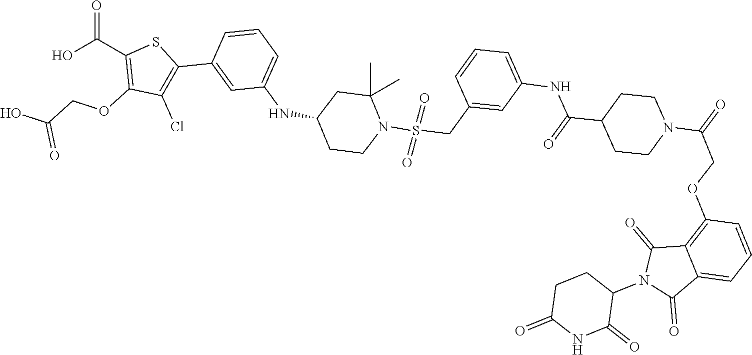 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)COc3cccc4c3C(=O)N(C3CCC(=O)NC3=O)C4=O)CC2)c1] |
| 59 | 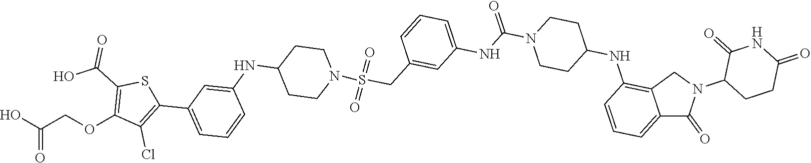 [SMILES: O=C(O)COc1c(C(=O)O)sc(-c2cccc(NC3CCN(S(=O)(=O)Cc4cccc(NC(=O)N5CCC(Nc6cccc7c6CN(C6CCC(=O)NC6=O)C7=O)CC5)c4)CC3)c2)c1Cl] |
| 60 | 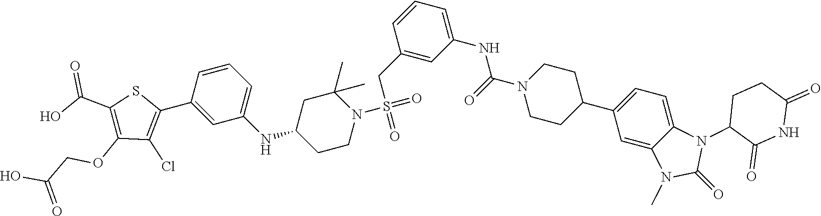 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 61 | 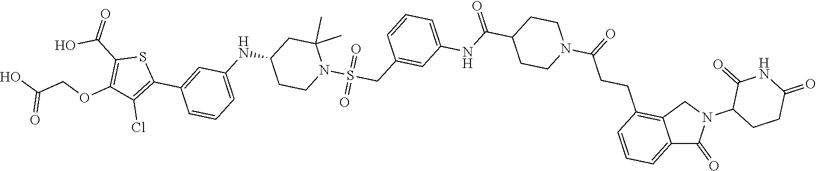 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CCc3cccc4c3CN(C3CCC(=O)NC3=O)C4=O)CC2)c1] |
| 62 | 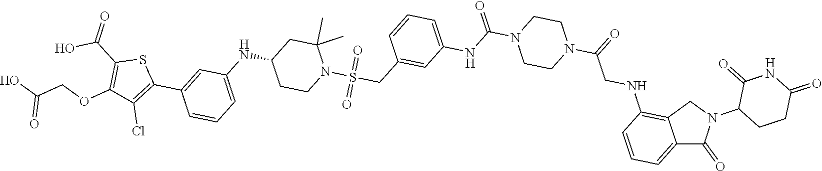 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCN(C(=O)CNc3cccc4c3CN(C3CCC(=O)NC3=O)C4=O)CC2)c1] |
| 63 | 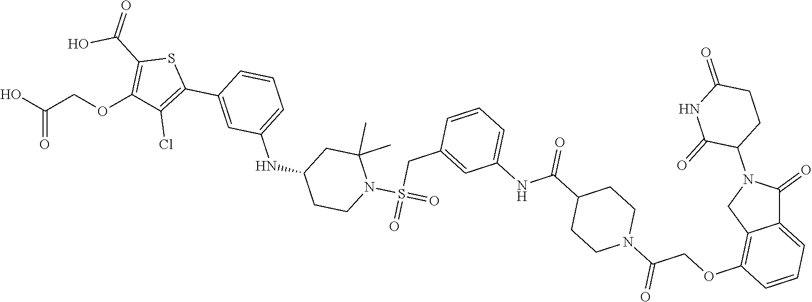 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)COc3cccc4c3CN(C3CCC(=O)NC3=O)C4=O)CC2)c1] |
| 64 | 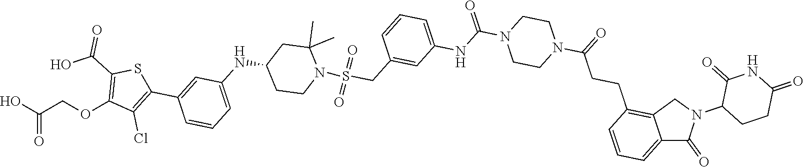 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCN(C(=O)CCc3cccc4c3CN(C3CCC(=O)NC3=O)C4=O)CC2)c1] |
| 65 | 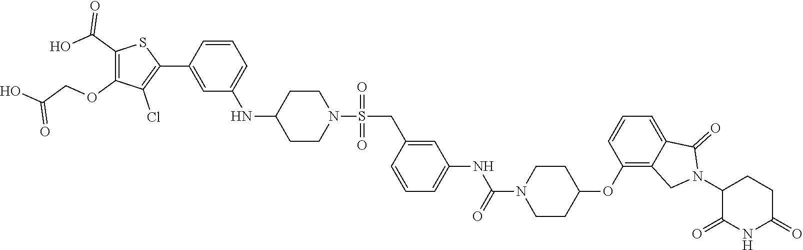 [SMILES: O=C(O)COc1c(C(=O)O)sc(-c2cccc(NC3CCN(S(=O)(=O)Cc4cccc(NC(=O)N5CCC(Oc6cccc7c6CN(C6CCC(=O)NC6=O)C7=O)CC5)c4)CC3)c2)c1Cl] |
| 66 | 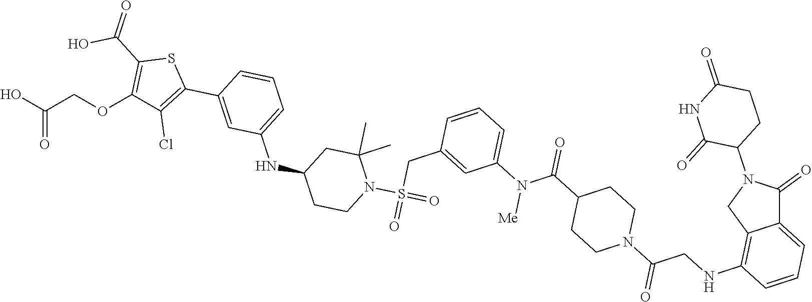 [SMILES: CN(C(=O)C1CCN(C(=O)CNc2cccc3c2CN(C2CCC(=O)NC2=O)C3=O)CC1)c1cccc(CS(=O)(=O)N2CC[C@@H](Nc3cccc(-c4sc(C(=O)O)c(OCC(=O)O)c4Cl)c3)CC2(C)C)c1] |
| 67 | 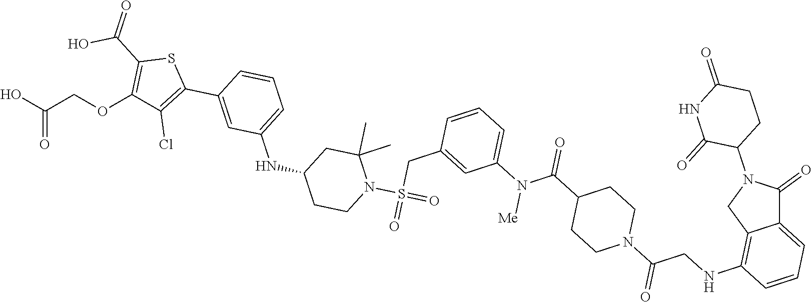 [SMILES: CN(C(=O)C1CCN(C(=O)CNc2cccc3c2CN(C2CCC(=O)NC2=O)C3=O)CC1)c1cccc(CS(=O)(=O)N2CC[C@H](Nc3cccc(-c4sc(C(=O)O)c(OCC(=O)O)c4Cl)c3)CC2(C)C)c1] |
| 68 | 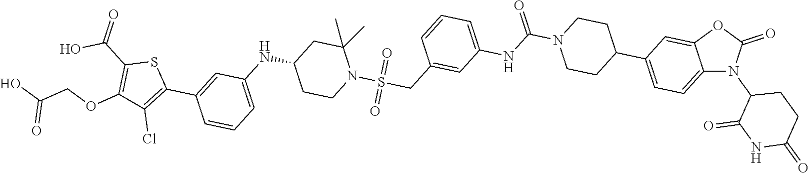 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)oc(=O)n4C3CCC(=O)NC3=O)CC2)c1] |
| 69 | 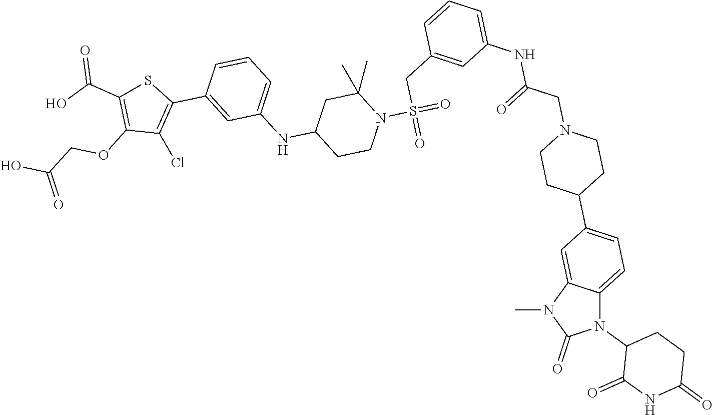 [SMILES: Cn1c(=O)n(C2CCC(=O)NC2=O)c2ccc(C3CCN(CC(=O)Nc4cccc(CS(=O)(=O)N5CCC(Nc6cccc(-c7sc(C(=O)O)c(OCC(=O)O)c7Cl)c6)CC5(C)C)c4)CC3)cc21] |
| 70 | 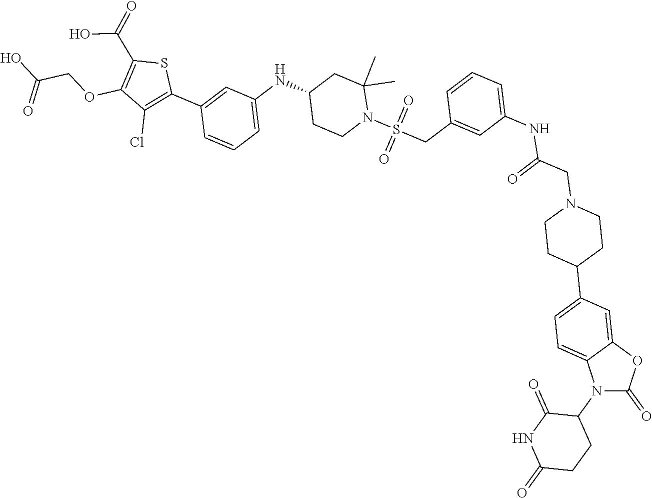 [SMILES: CC1(C)C[C@@H](Nc2cccc(-c3sc(C(=O)O)c(OCC(=O)O)c3Cl)c2)CCN1S(=O)(=O)Cc1cccc(NC(=O)CN2CCC(c3ccc4c(c3)oc(=O)n4C3CCC(=O)NC3=O)CC2)c1] |
| 71 | 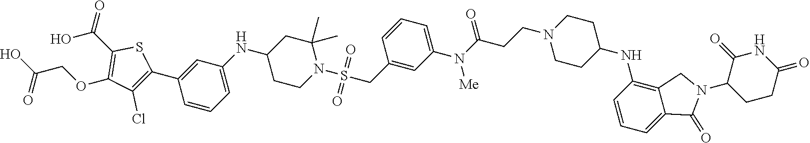 [SMILES: CN(C(=O)CCN1CCC(Nc2cccc3c2CN(C2CCC(=O)NC2=O)C3=O)CC1)c1cccc(CS(=O)(=O)N2CCC(Nc3cccc(-c4sc(C(=O)O)c(OCC(=O)O)c4Cl)c3)CC2(C)C)c1] |
| 72 | 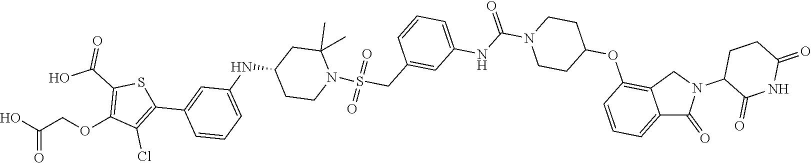 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(Oc3cccc4c3CN(C3CCC(=O)NC3=O)C4=O)CC2)c1] |
| 73 | 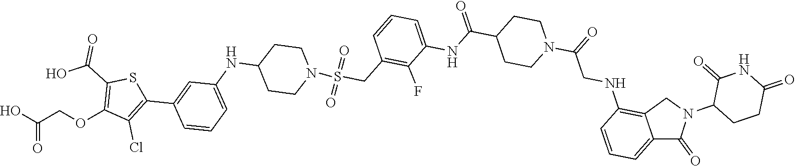 [SMILES: O=C(O)COc1c(C(=O)O)sc(-c2cccc(NC3CCN(S(=O)(=O)Cc4cccc(NC(=O)C5CCN(C(=O)CNc6cccc7c6CN(C6CCC(=O)NC6=O)C7=O)CC5)c4F)CC3)c2)c1Cl] |
| 74 | 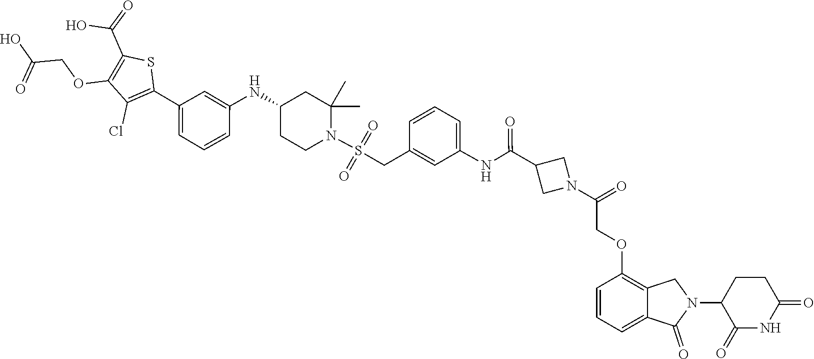 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)COc3cccc4c3CN(C3CCC(=O)NC3=O)C4=O)C2)c1] |
| 75 | 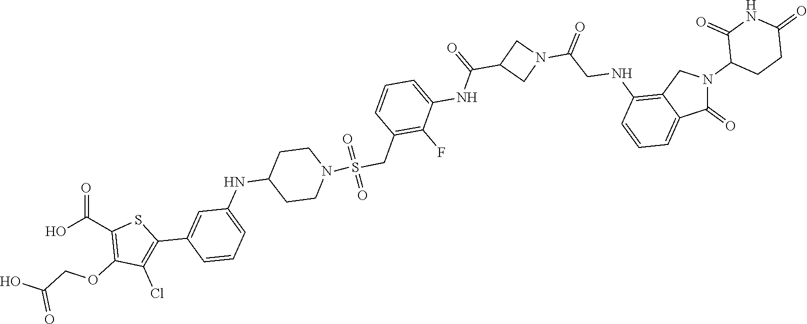 [SMILES: O=C(O)COc1c(C(=O)O)sc(-c2cccc(NC3CCN(S(=O)(=O)Cc4cccc(NC(=O)C5CN(C(=O)CNc6cccc7c6CN(C6CCC(=O)NC6=O)C7=O)C5)c4F)CC3)c2)c1Cl] |
| 76 | 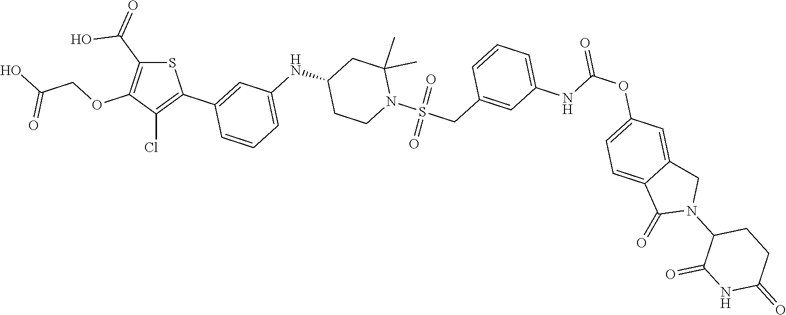 [SMILES: CC1(C)C[C@@H](Nc2cccc(-c3sc(C(=O)O)c(OCC(=O)O)c3Cl)c2)CCN1S(=O)(=O)Cc1cccc(NC(=O)Oc2ccc3c(c2)CN(C2CCC(=O)NC2=O)C3=O)c1] |
| 77 | 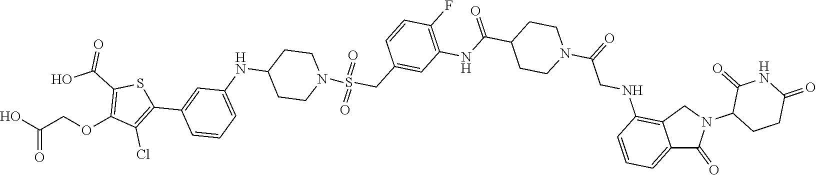 [SMILES: O=C(O)COc1c(C(=O)O)sc(-c2cccc(NC3CCN(S(=O)(=O)Cc4ccc(F)c(NC(=O)C5CCN(C(=O)CNc6cccc7c6CN(C6CCC(=O)NC6=O)C7=O)CC5)c4)CC3)c2)c1Cl] |
| 78 | 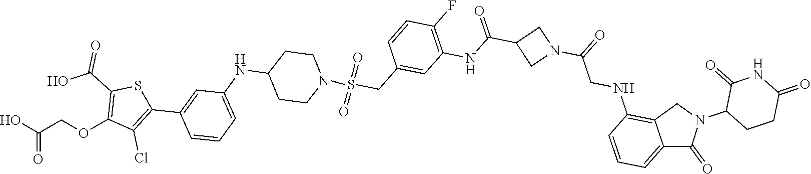 [SMILES: O=C(O)COc1c(C(=O)O)sc(-c2cccc(NC3CCN(S(=O)(=O)Cc4ccc(F)c(NC(=O)C5CN(C(=O)CNc6cccc7c6CN(C6CCC(=O)NC6=O)C7=O)C5)c4)CC3)c2)c1Cl] |
| 79 | 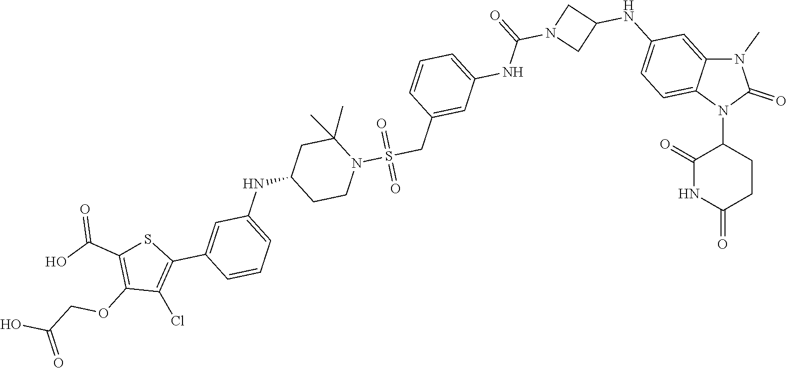 [SMILES: Cn1c(=O)n(C2CCC(=O)NC2=O)c2ccc(NC3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 80 | 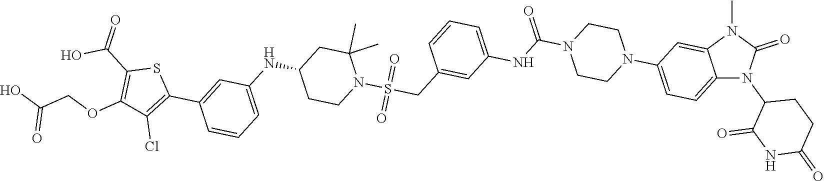 [SMILES: Cn1c(=O)n(C2CCC(=O)NC2=O)c2ccc(N3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 81 | 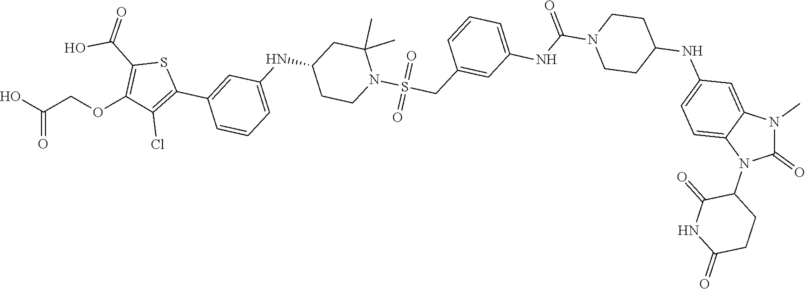 [SMILES: Cn1c(=O)n(C2CCC(=O)NC2=O)c2ccc(NC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 82 | 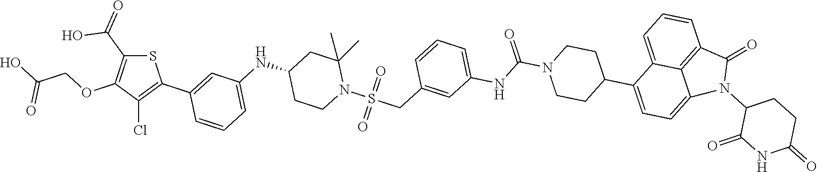 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1] |
| 83 | 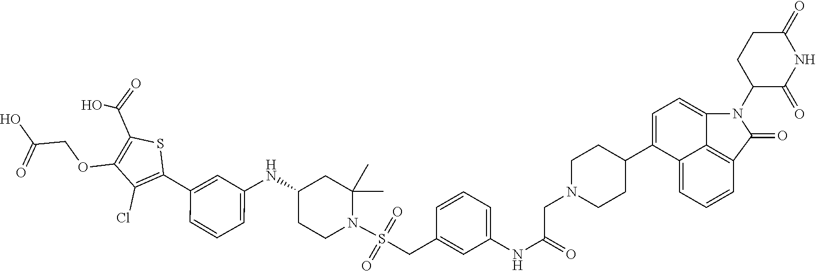 [SMILES: CC1(C)C[C@@H](Nc2cccc(-c3sc(C(=O)O)c(OCC(=O)O)c3Cl)c2)CCN1S(=O)(=O)Cc1cccc(NC(=O)CN2CCC(c3ccc4c5c(cccc35)C(=O)N4C3CCC(=O)NC3=O)CC2)c1] |
| 84 | 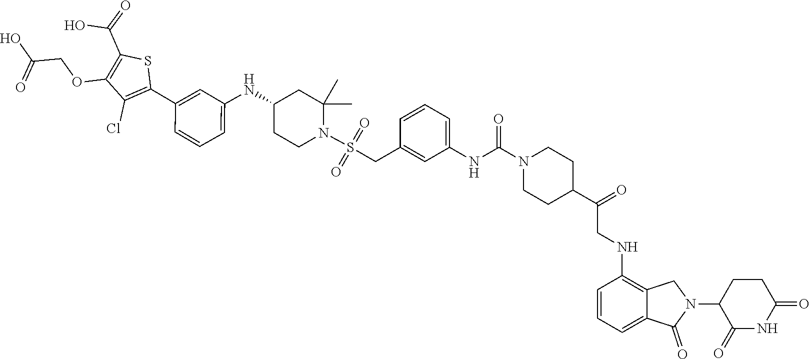 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(C(=O)CNc3cccc4c3CN(C3CCC(=O)NC3=O)C4=O)CC2)c1] |
| 85 | 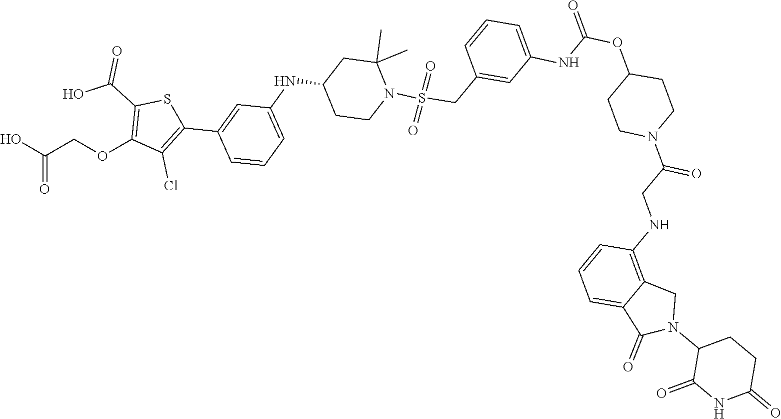 [SMILES: CC1(C)C[C@@H](Nc2cccc(-c3sc(C(=O)O)c(OCC(=O)O)c3Cl)c2)CCN1S(=O)(=O)Cc1cccc(NC(=O)OC2CCN(C(=O)CNc3cccc4c3CN(C3CCC(=O)NC3=O)C4=O)CC2)c1] |
| 86 | 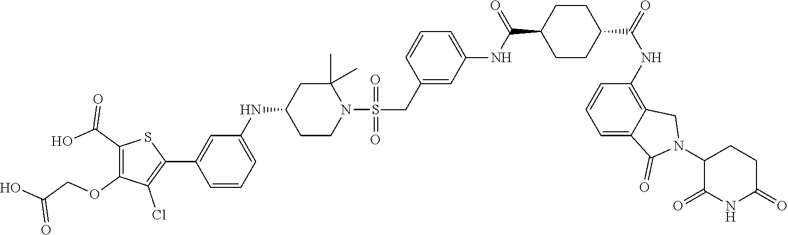 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@H](C(=O)Nc3cccc4c3CN(C3CCC(=O)NC3=O)C4=O)CC2)c1] |
| 87 | 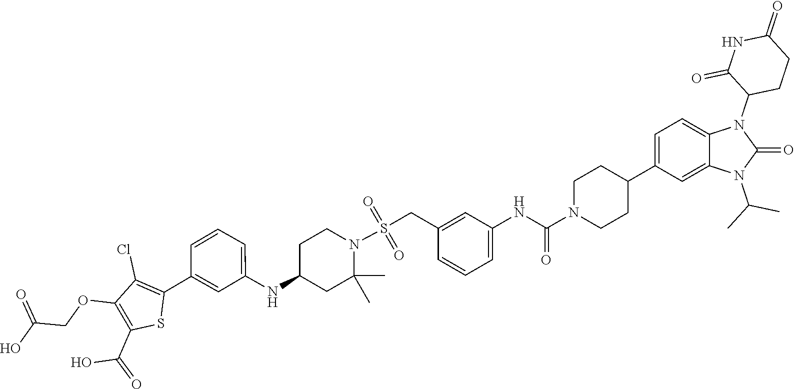 [SMILES: CC(C)n1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 87a | 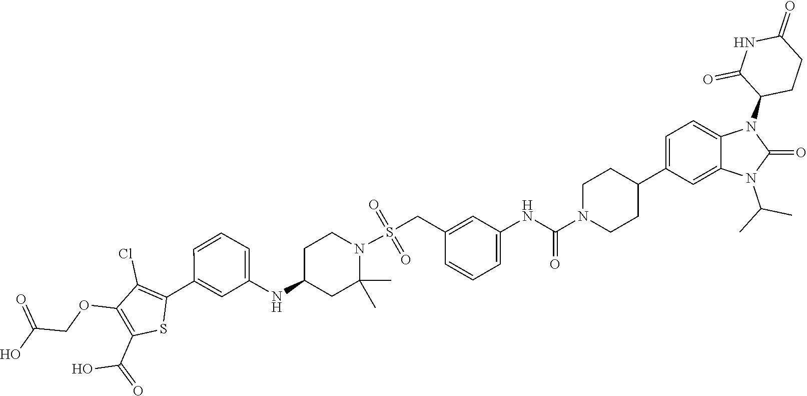 [SMILES: CC(C)n1c(=O)n([C@@H]2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 87b | 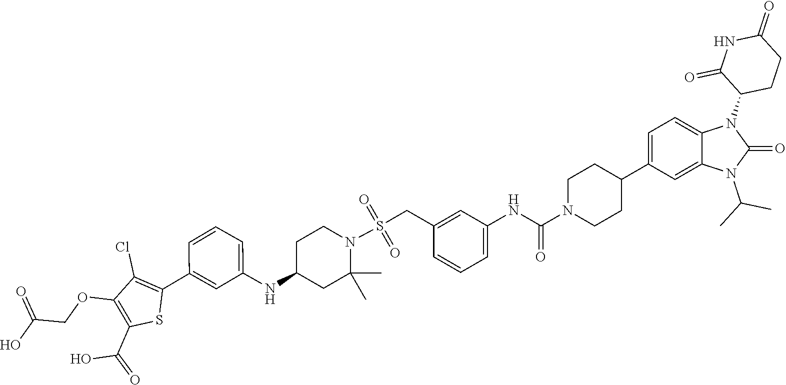 [SMILES: CC(C)n1c(=O)n([C@H]2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 88 | 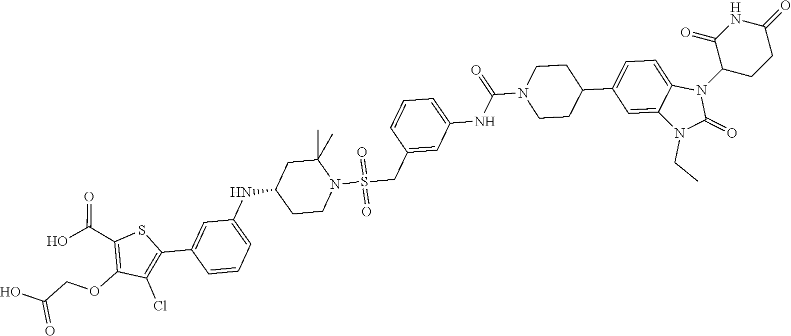 [SMILES: CCn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 89 | 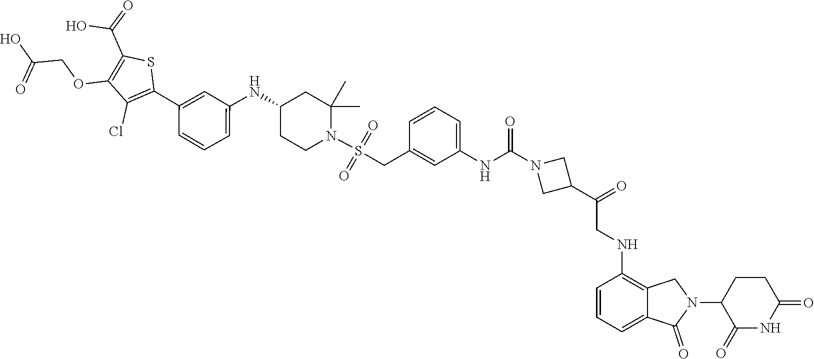 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CC(C(=O)CNc3cccc4c3CN(C3CCC(=O)NC3=O)C4=O)C2)c1] |
| 90 | 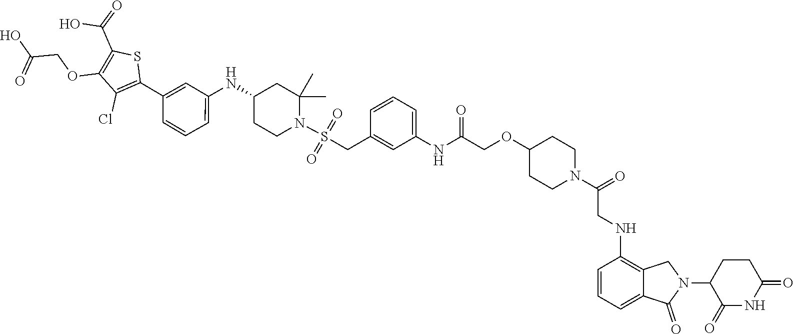 [SMILES: CC1(C)C[C@@H](Nc2cccc(-c3sc(C(=O)O)c(OCC(=O)O)c3Cl)c2)CCN1S(=O)(=O)Cc1cccc(NC(=O)COC2CCN(C(=O)CNc3cccc4c3CN(C3CCC(=O)NC3=O)C4=O)CC2)c1] |
| 91 | 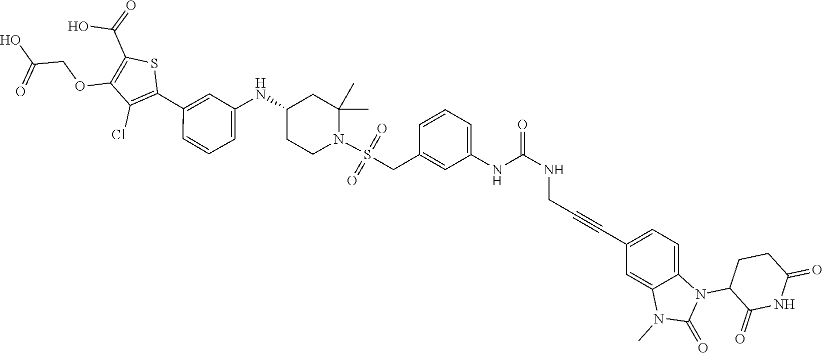 [SMILES: Cn1c(=O)n(C2CCC(=O)NC2=O)c2ccc(C#CCNC(=O)Nc3cccc(CS(=O)(=O)N4CC[C@H](Nc5cccc(-c6sc(C(=O)O)c(OCC(=O)O)c6Cl)c5)CC4(C)C)c3)cc21] |
| 92 | 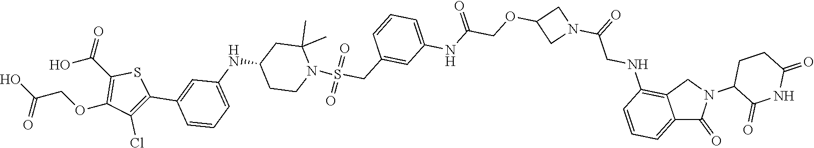 [SMILES: CC1(C)C[C@@H](Nc2cccc(-c3sc(C(=O)O)c(OCC(=O)O)c3Cl)c2)CCN1S(=O)(=O)Cc1cccc(NC(=O)COC2CN(C(=O)CNc3cccc4c3CN(C3CCC(=O)NC3=O)C4=O)C2)c1] |
| 93 | 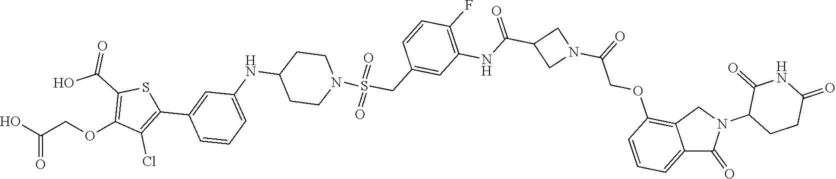 [SMILES: O=C(O)COc1c(C(=O)O)sc(-c2cccc(NC3CCN(S(=O)(=O)Cc4ccc(F)c(NC(=O)C5CN(C(=O)COc6cccc7c6CN(C6CCC(=O)NC6=O)C7=O)C5)c4)CC3)c2)c1Cl] |
| 94 | 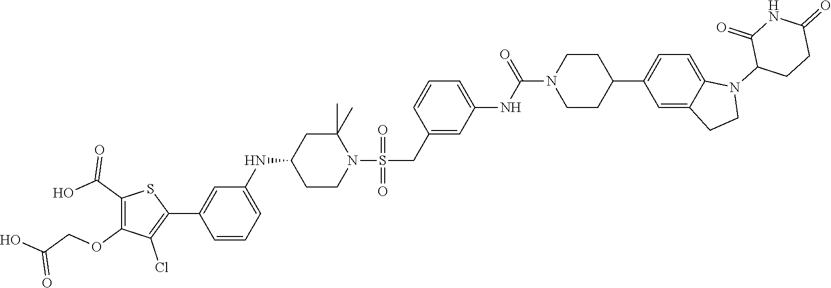 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)CCN4C3CCC(=O)NC3=O)CC2)c1] |
| 95 | 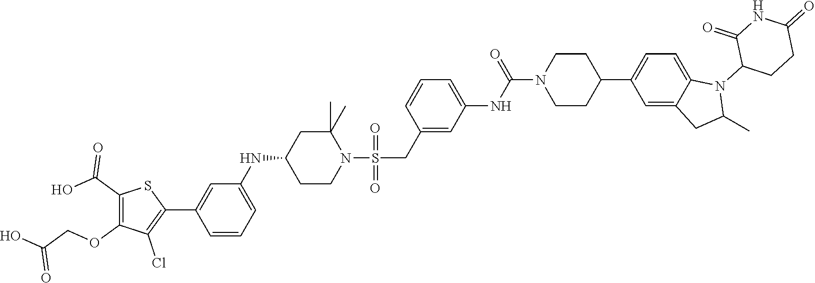 [SMILES: CC1Cc2cc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)ccc2N1C1CCC(=O)NC1=O] |
| 96 | 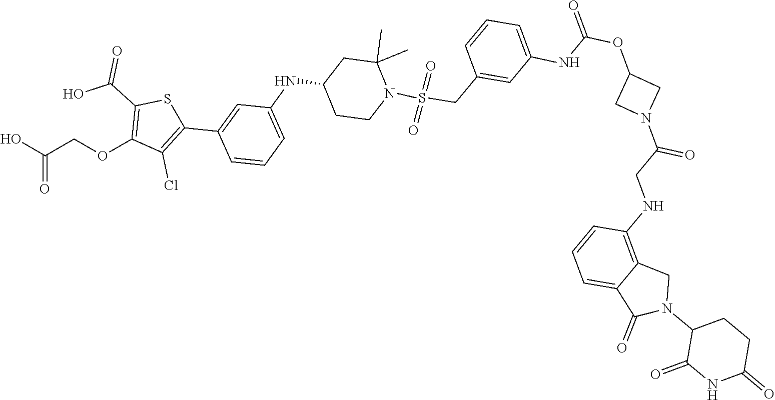 [SMILES: CC1(C)C[C@@H](Nc2cccc(-c3sc(C(=O)O)c(OCC(=O)O)c3Cl)c2)CCN1S(=O)(=O)Cc1cccc(NC(=O)OC2CN(C(=O)CNc3cccc4c3CN(C3CCC(=O)NC3=O)C4=O)C2)c1] |
| 97 | 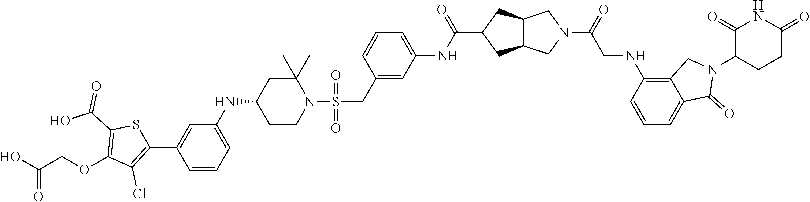 |
| 98 | 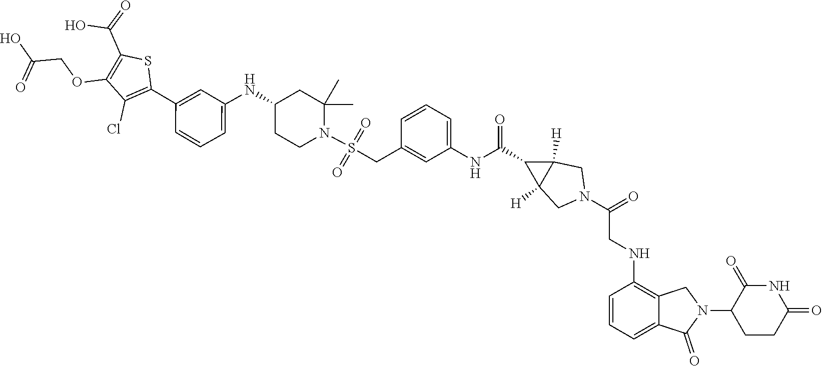 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2[C@@H]3CN(C(=O)CNc4cccc5c4CN(C4CCC(=O)NC4=O)C5=O)C[C@@H]32)c1] |
| 99 | 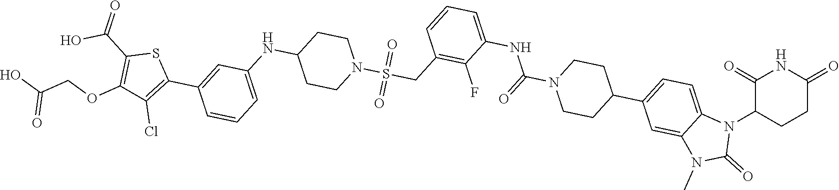 [SMILES: Cn1c(=O)n(C2CCC(=O)NC2=O)c2ccc(C3CCN(C(=O)Nc4cccc(CS(=O)(=O)N5CCC(Nc6cccc(-c7sc(C(=O)O)c(OCC(=O)O)c7Cl)c6)CC5)c4F)CC3)cc21] |
| 100 | 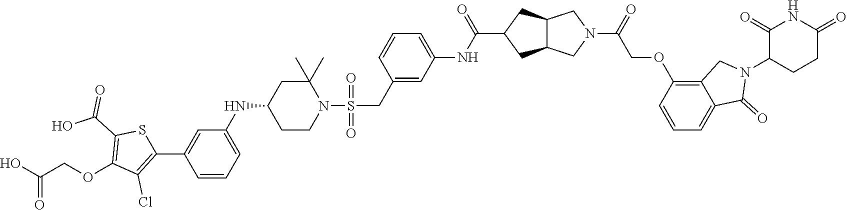 |
| 101 | 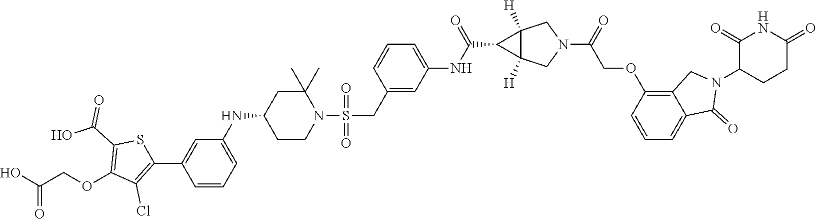 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2[C@@H]3CN(C(=O)COc4cccc5c4CN(C4CCC(=O)NC4=O)C5=O)C[C@@H]32)c1] |
| 102 | 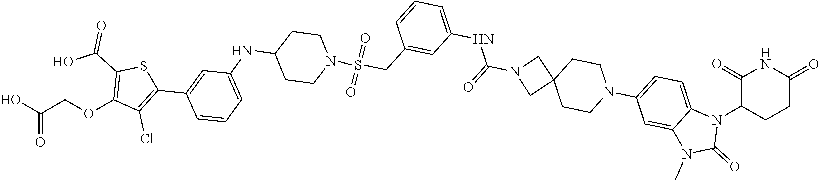 [SMILES: Cn1c(=O)n(C2CCC(=O)NC2=O)c2ccc(N3CCC4(CC3)CN(C(=O)Nc3cccc(CS(=O)(=O)N5CCC(Nc6cccc(-c7sc(C(=O)O)c(OCC(=O)O)c7Cl)c6)CC5)c3)C4)cc21] |
| 103 | 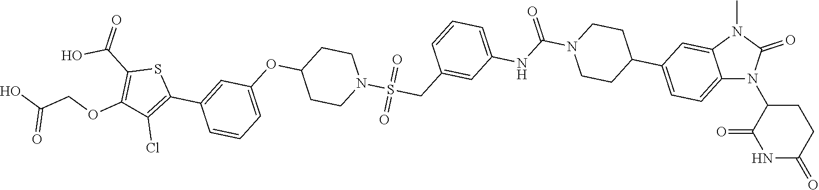 [SMILES: Cn1c(=O)n(C2CCC(=O)NC2=O)c2ccc(C3CCN(C(=O)Nc4cccc(CS(=O)(=O)N5CCC(Oc6cccc(-c7sc(C(=O)O)c(OCC(=O)O)c7Cl)c6)CC5)c4)CC3)cc21] |
| 104 | 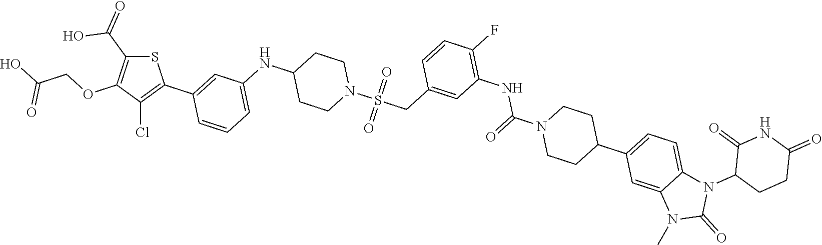 [SMILES: Cn1c(=O)n(C2CCC(=O)NC2=O)c2ccc(C3CCN(C(=O)Nc4cc(CS(=O)(=O)N5CCC(Nc6cccc(-c7sc(C(=O)O)c(OCC(=O)O)c7Cl)c6)CC5)ccc4F)CC3)cc21] |
| 105 | 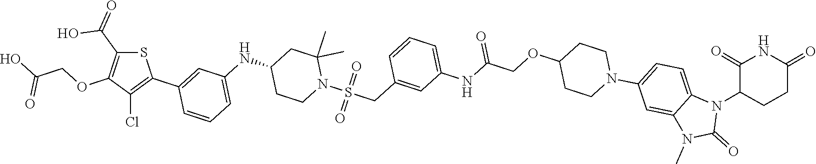 [SMILES: Cn1c(=O)n(C2CCC(=O)NC2=O)c2ccc(N3CCC(OCC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 106 | 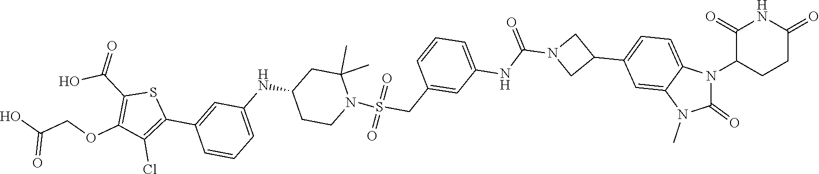 [SMILES: Cn1c(=O)n(C2CCC(=O)NC2=O)c2ccc(C3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 107 | 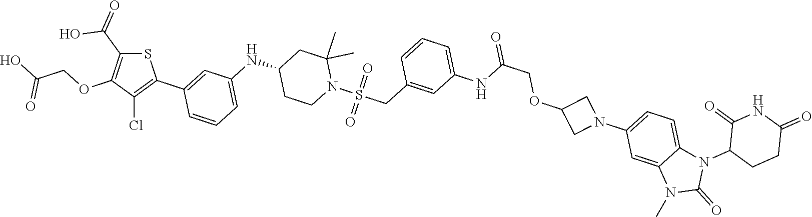 [SMILES: Cn1c(=O)n(C2CCC(=O)NC2=O)c2ccc(N3CC(OCC(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 108 | 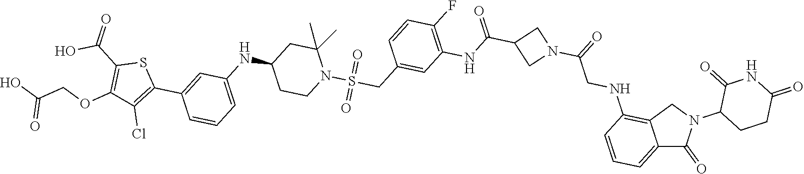 [SMILES: CC1(C)C[C@H](Nc2cccc(-c3sc(C(=O)O)c(OCC(=O)O)c3Cl)c2)CCN1S(=O)(=O)Cc1ccc(F)c(NC(=O)C2CN(C(=O)CNc3cccc4c3CN(C3CCC(=O)NC3=O)C4=O)C2)c1] |
| 109 | 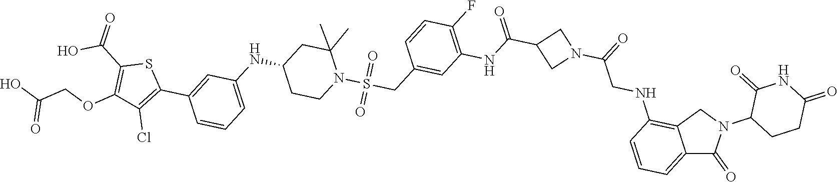 [SMILES: CC1(C)C[C@@H](Nc2cccc(-c3sc(C(=O)O)c(OCC(=O)O)c3Cl)c2)CCN1S(=O)(=O)Cc1ccc(F)c(NC(=O)C2CN(C(=O)CNc3cccc4c3CN(C3CCC(=O)NC3=O)C4=O)C2)c1] |
| 110 | 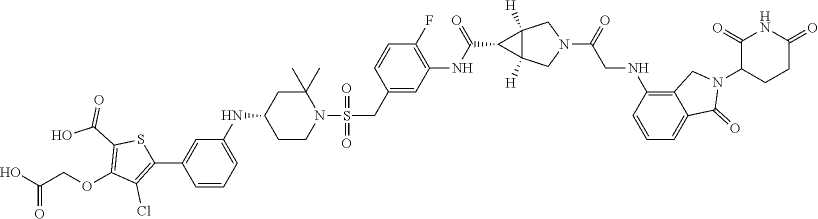 [SMILES: CC1(C)C[C@@H](Nc2cccc(-c3sc(C(=O)O)c(OCC(=O)O)c3Cl)c2)CCN1S(=O)(=O)Cc1ccc(F)c(NC(=O)[C@H]2[C@@H]3CN(C(=O)CNc4cccc5c4CN(C4CCC(=O)NC4=O)C5=O)C[C@@H]32)c1] |
| 111 | 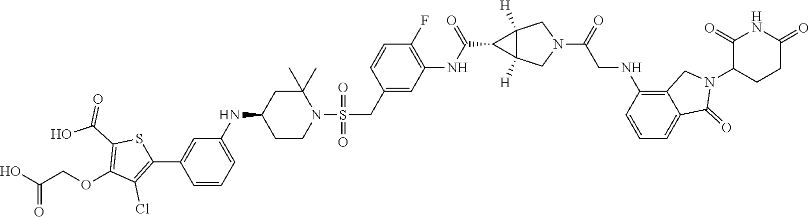 [SMILES: CC1(C)C[C@H](Nc2cccc(-c3sc(C(=O)O)c(OCC(=O)O)c3Cl)c2)CCN1S(=O)(=O)Cc1ccc(F)c(NC(=O)[C@H]2[C@@H]3CN(C(=O)CNc4cccc5c4CN(C4CCC(=O)NC4=O)C5=O)C[C@@H]32)c1] |
| 112 | 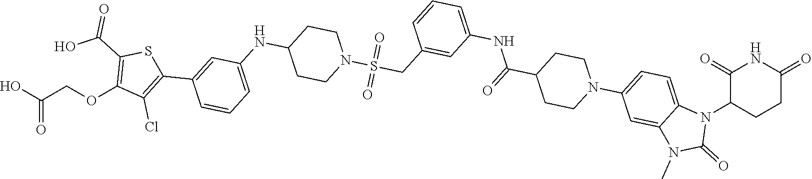 [SMILES: Cn1c(=O)n(C2CCC(=O)NC2=O)c2ccc(N3CCC(C(=O)Nc4cccc(CS(=O)(=O)N5CCC(Nc6cccc(-c7sc(C(=O)O)c(OCC(=O)O)c7Cl)c6)CC5)c4)CC3)cc21] |
| 113 | 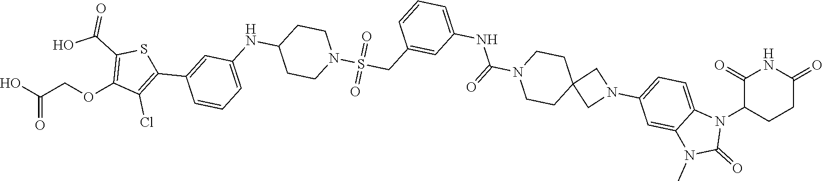 [SMILES: Cn1c(=O)n(C2CCC(=O)NC2=O)c2ccc(N3CC4(CCN(C(=O)Nc5cccc(CS(=O)(=O)N6CCC(Nc7cccc(-c8sc(C(=O)O)c(OCC(=O)O)c8Cl)c7)CC6)c5)CC4)C3)cc21] |
| 114 | 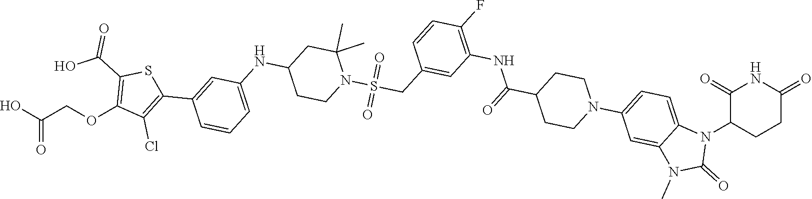 [SMILES: Cn1c(=O)n(C2CCC(=O)NC2=O)c2ccc(N3CCC(C(=O)Nc4cc(CS(=O)(=O)N5CCC(Nc6cccc(-c7sc(C(=O)O)c(OCC(=O)O)c7Cl)c6)CC5(C)C)ccc4F)CC3)cc21] |
| 115 | 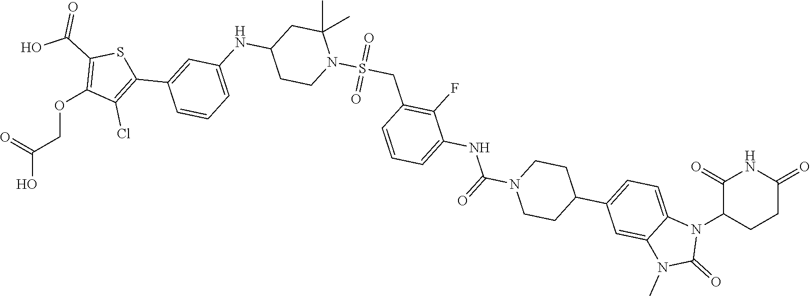 [SMILES: Cn1c(=O)n(C2CCC(=O)NC2=O)c2ccc(C3CCN(C(=O)Nc4cccc(CS(=O)(=O)N5CCC(Nc6cccc(-c7sc(C(=O)O)c(OCC(=O)O)c7Cl)c6)CC5(C)C)c4F)CC3)cc21] |
| 116 | 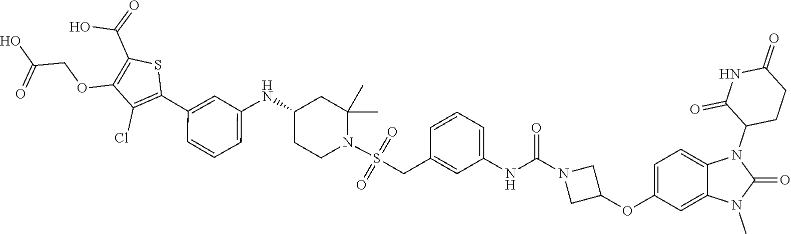 [SMILES: Cn1c(=O)n(C2CCC(=O)NC2=O)c2ccc(OC3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 117 | 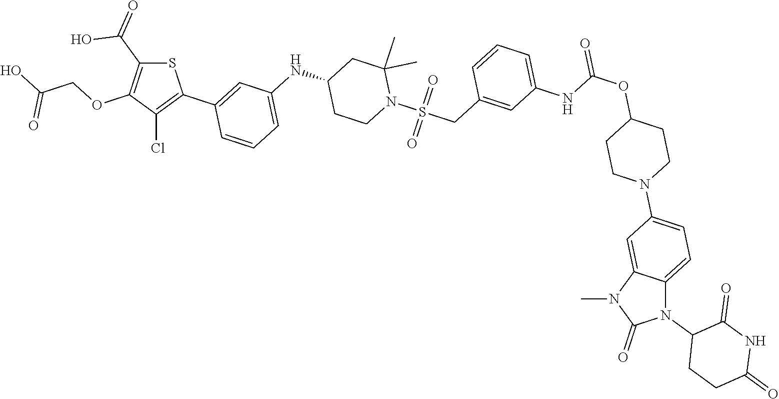 [SMILES: Cn1c(=O)n(C2CCC(=O)NC2=O)c2ccc(N3CCC(OC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 118 | 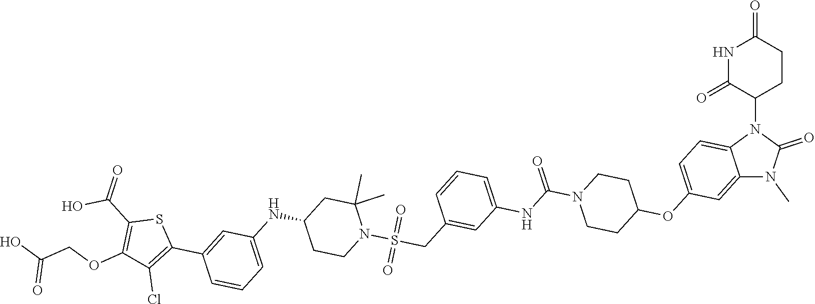 [SMILES: Cn1c(=O)n(C2CCC(=O)NC2=O)c2ccc(OC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 119 | 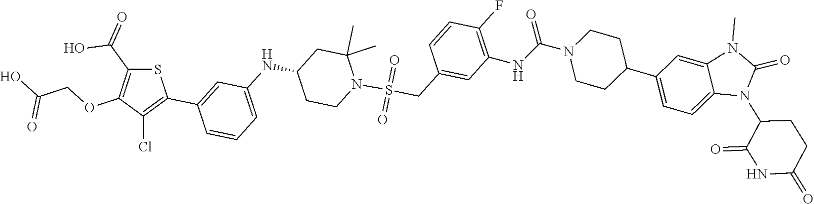 [SMILES: Cn1c(=O)n(C2CCC(=O)NC2=O)c2ccc(C3CCN(C(=O)Nc4cc(CS(=O)(=O)N5CC[C@H](Nc6cccc(-c7sc(C(=O)O)c(OCC(=O)O)c7Cl)c6)CC5(C)C)ccc4F)CC3)cc21] |
| 120 | 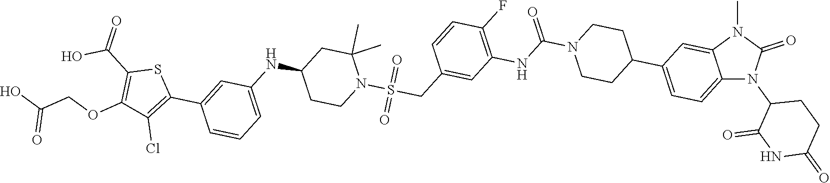 [SMILES: Cn1c(=O)n(C2CCC(=O)NC2=O)c2ccc(C3CCN(C(=O)Nc4cc(CS(=O)(=O)N5CC[C@@H](Nc6cccc(-c7sc(C(=O)O)c(OCC(=O)O)c7Cl)c6)CC5(C)C)ccc4F)CC3)cc21] |
| 121 | 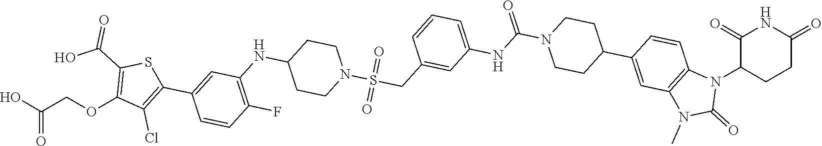 [SMILES: Cn1c(=O)n(C2CCC(=O)NC2=O)c2ccc(C3CCN(C(=O)Nc4cccc(CS(=O)(=O)N5CCC(Nc6cc(-c7sc(C(=O)O)c(OCC(=O)O)c7Cl)ccc6F)CC5)c4)CC3)cc21] |
| 122 | 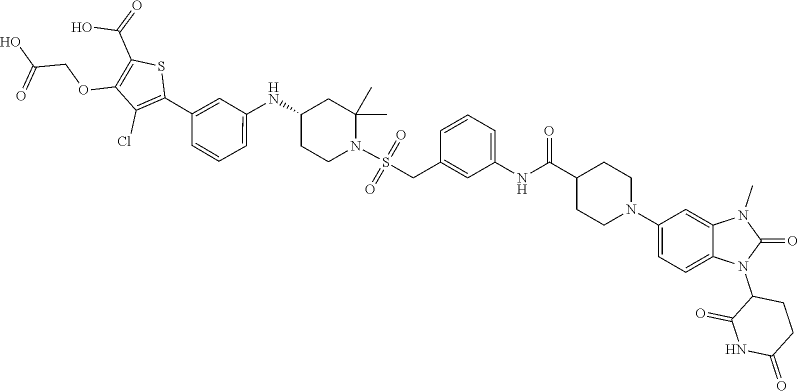 [SMILES: Cn1c(=O)n(C2CCC(=O)NC2=O)c2ccc(N3CCC(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 123 | 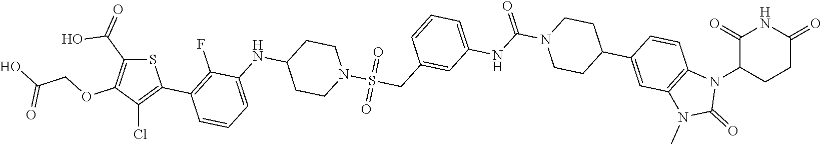 [SMILES: Cn1c(=O)n(C2CCC(=O)NC2=O)c2ccc(C3CCN(C(=O)Nc4cccc(CS(=O)(=O)N5CCC(Nc6cccc(-c7sc(C(=O)O)c(OCC(=O)O)c7Cl)c6F)CC5)c4)CC3)cc21] |
| 124 | 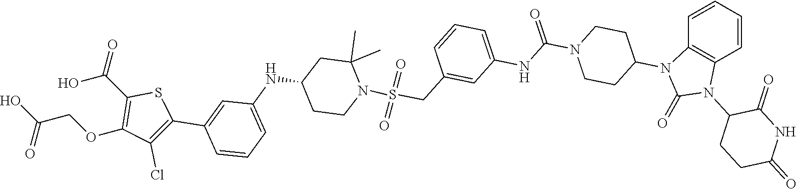 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(n3c(=O)n(C4CCC(=O)NC4=O)c4ccccc43)CC2)c1] |
| 125 | 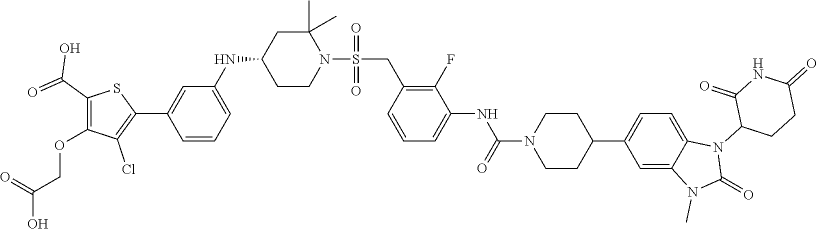 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4F)CC3)cc21] |
| 126 | 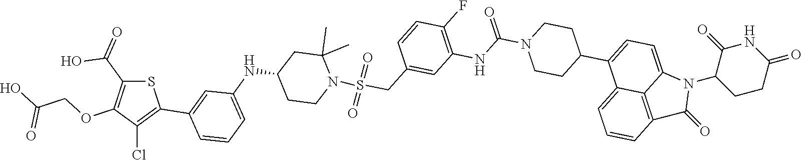 [SMILES: CC1(C)C[C@@H](Nc2cccc(-c3sc(C(=O)O)c(OCC(=O)O)c3Cl)c2)CCN1S(=O)(=O)Cc1ccc(F)c(NC(=O)N2CCC(c3ccc4c5c(cccc35)C(=O)N4C3CCC(=O)NC3=O)CC2)c1] |
| 127 | 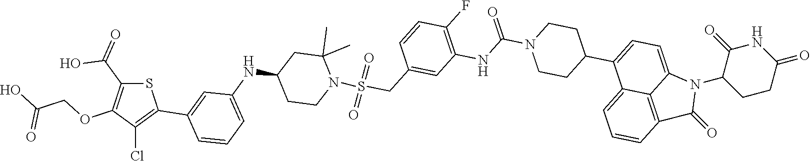 [SMILES: CC1(C)C[C@H](Nc2cccc(-c3sc(C(=O)O)c(OCC(=O)O)c3Cl)c2)CCN1S(=O)(=O)Cc1ccc(F)c(NC(=O)N2CCC(c3ccc4c5c(cccc35)C(=O)N4C3CCC(=O)NC3=O)CC2)c1] |
| 128 | 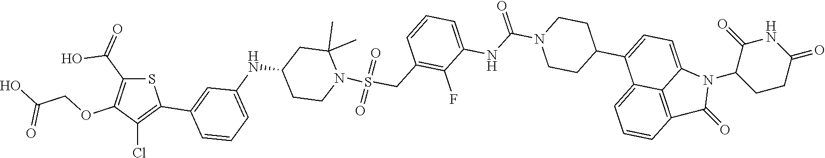 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1F] |
| 129 | 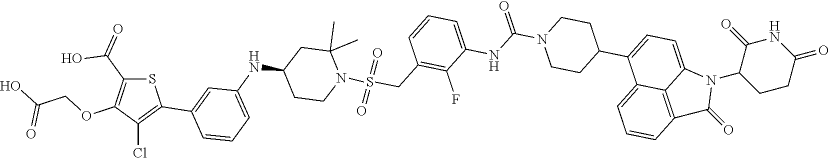 [SMILES: CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1F] |
| 130 | 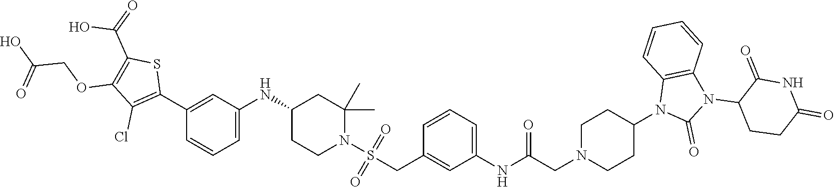 [SMILES: CC1(C)C[C@@H](Nc2cccc(-c3sc(C(=O)O)c(OCC(=O)O)c3Cl)c2)CCN1S(=O)(=O)Cc1cccc(NC(=O)CN2CCC(n3c(=O)n(C4CCC(=O)NC4=O)c4ccccc43)CC2)c1] |
| 131 | 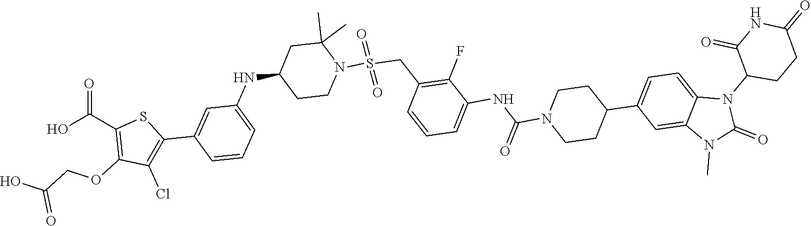 [SMILES: Cn1c(=O)n(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6)CC5(C)C)c4F)CC3)cc21] |
| 132 | 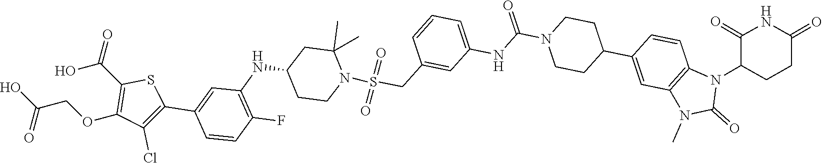 [SMILES: Cn1c(=O)n(C2CCC(=O)NC2=O)c2ccc(C3CCN(C(=O)Nc4cccc(CS(=O)(=O)N5CC[C@H](Nc6cc(-c7sc(C(=O)O)c(OCC(=O)O)c7Cl)ccc6F)CC5(C)C)c4)CC3)cc21] |
| 133 | 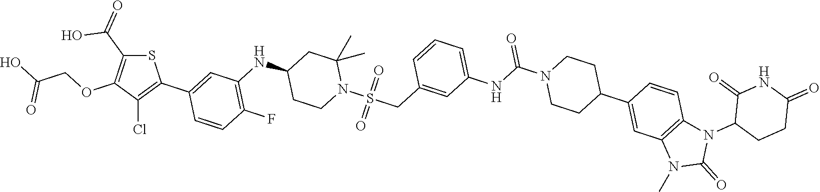 [SMILES: Cn1c(=O)n(C2CCC(=O)NC2=O)c2ccc(C3CCN(C(=O)Nc4cccc(CS(=O)(=O)N5CC[C@@H](Nc6cc(-c7sc(C(=O)O)c(OCC(=O)O)c7Cl)ccc6F)CC5(C)C)c4)CC3)cc21] |
| 134 | 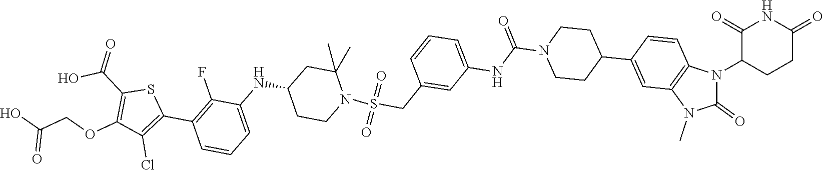 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6F)CC5(C)C)c4)CC3)cc21] |
| 135 | 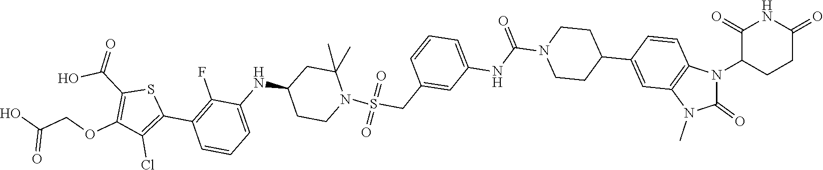 [SMILES: Cn1c(=O)n(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6F)CC5(C)C)c4)CC3)cc21] |
| 136 | 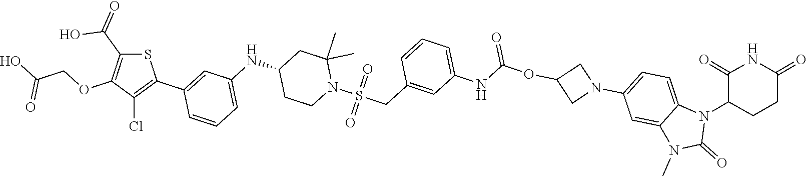 [SMILES: Cn1c(=O)n(C2CCC(=O)NC2=O)c2ccc(N3CC(OC(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 137 | 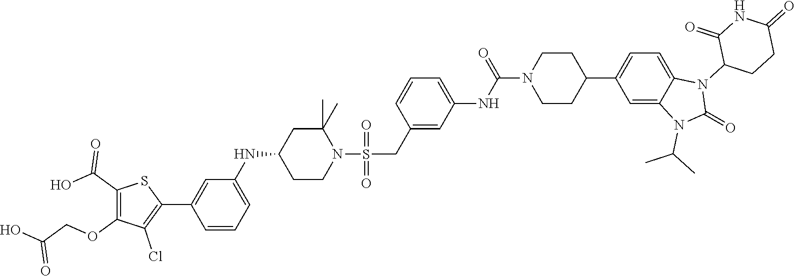 [SMILES: CC(C)n1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 138 | 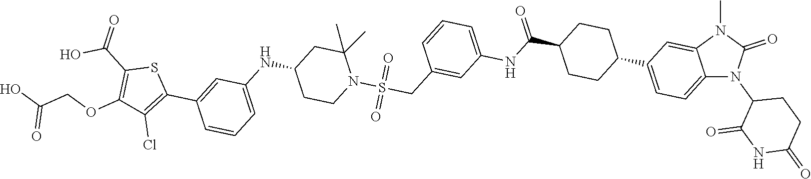 [SMILES: Cn1c(=O)n(C2CCC(=O)NC2=O)c2ccc([C@H]3CC[C@H](C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 139 | 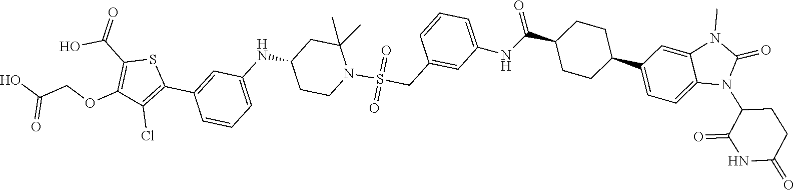 [SMILES: Cn1c(=O)n(C2CCC(=O)NC2=O)c2ccc([C@H]3CC[C@@H](C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 140 | 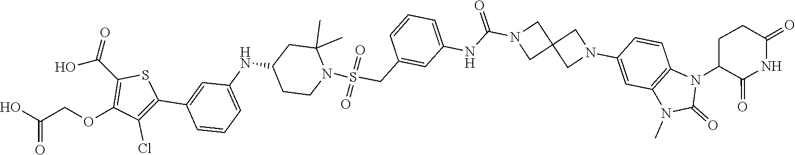 |
| 141 | 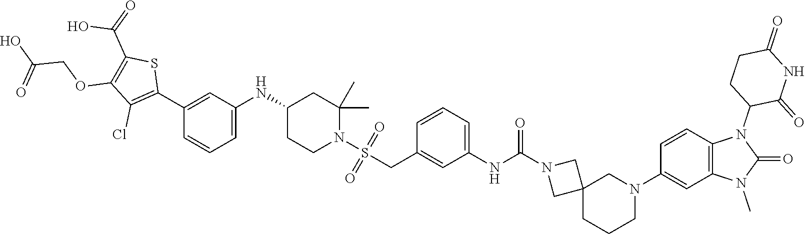 [SMILES: Cn1c(=O)n(C2CCC(=O)NC2=O)c2ccc(N3CCCC4(CN(C(=O)Nc5cccc(CS(=O)(=O)N6CC[C@H](Nc7cccc(-c8sc(C(=O)O)c(OCC(=O)O)c8Cl)c7)CC6(C)C)c5)C4)C3)cc21] |
| 142 | 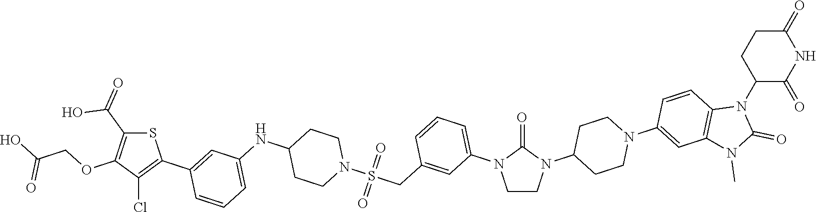 [SMILES: Cn1c(=O)n(C2CCC(=O)NC2=O)c2ccc(N3CCC(N4CCN(c5cccc(CS(=O)(=O)N6CCC(Nc7cccc(-c8sc(C(=O)O)c(OCC(=O)O)c8Cl)c7)CC6)c5)C4=O)CC3)cc21] |
| 143 | 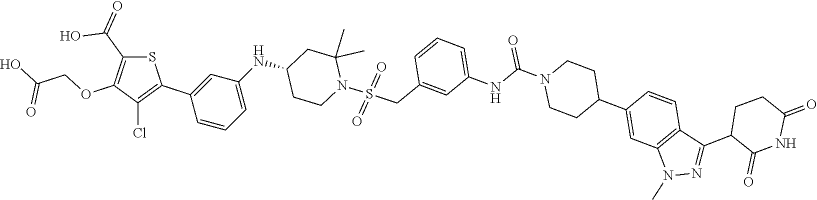 [SMILES: Cn1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 144 | 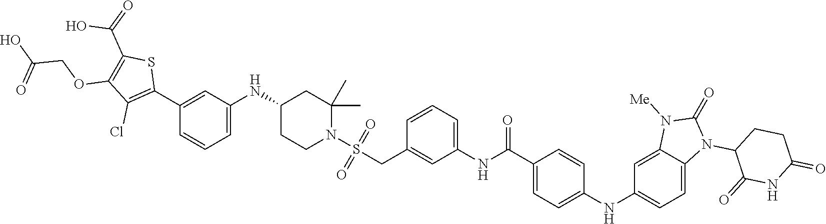 [SMILES: Cn1c(=O)n(C2CCC(=O)NC2=O)c2ccc(Nc3ccc(C(=O)Nc4cccc(CS(=O)(=O)N5CC[C@H](Nc6cccc(-c7sc(C(=O)O)c(OCC(=O)O)c7Cl)c6)CC5(C)C)c4)cc3)cc21] |
| 145 | 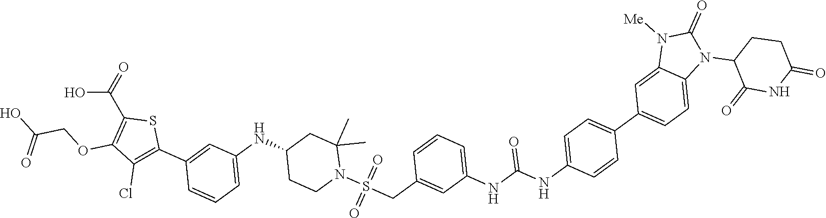 [SMILES: Cn1c(=O)n(C2CCC(=O)NC2=O)c2ccc(-c3ccc(NC(=O)Nc4cccc(CS(=O)(=O)N5CC[C@H](Nc6cccc(-c7sc(C(=O)O)c(OCC(=O)O)c7Cl)c6)CC5(C)C)c4)cc3)cc21] |
| 146 | 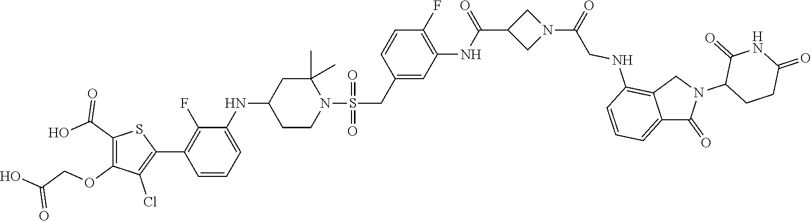 [SMILES: CC1(C)CC(Nc2cccc(-c3sc(C(=O)O)c(OCC(=O)O)c3Cl)c2F)CCN1S(=O)(=O)Cc1ccc(F)c(NC(=O)C2CN(C(=O)CNc3cccc4c3CN(C3CCC(=O)NC3=O)C4=O)C2)c1] |
| 147 | 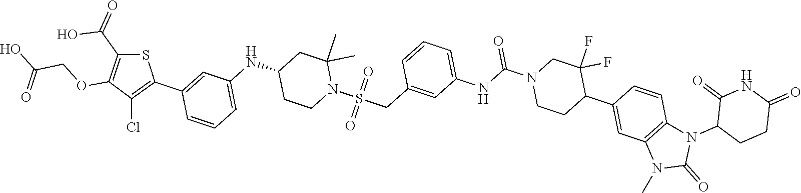 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 148 | 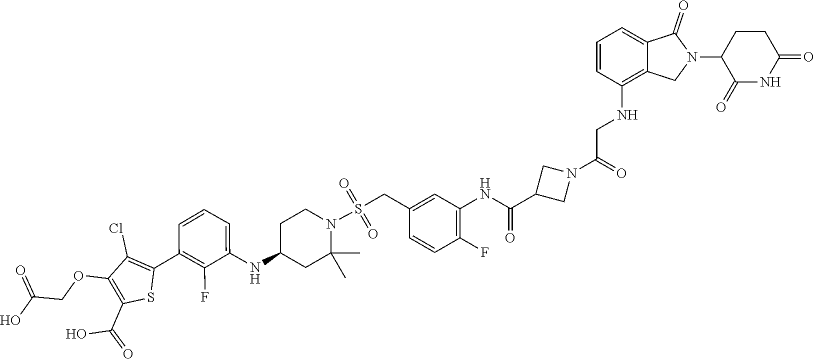 [SMILES: CC1(C)C[C@@H](Nc2cccc(-c3sc(C(=O)O)c(OCC(=O)O)c3Cl)c2F)CCN1S(=O)(=O)Cc1ccc(F)c(NC(=O)C2CN(C(=O)CNc3cccc4c3CN(C3CCC(=O)NC3=O)C4=O)C2)c1] |
| 148a | 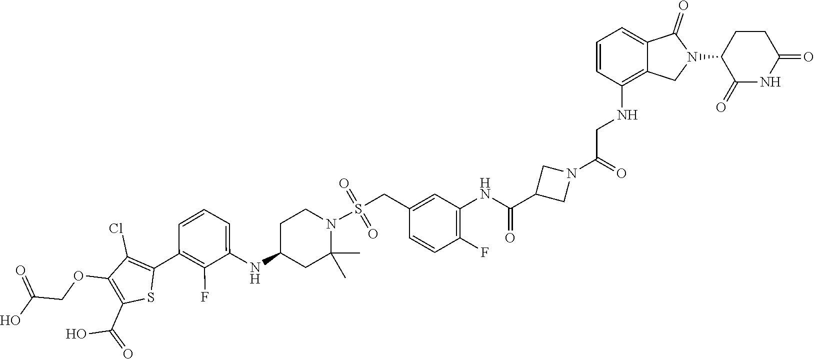 [SMILES: CC1(C)C[C@@H](Nc2cccc(-c3sc(C(=O)O)c(OCC(=O)O)c3Cl)c2F)CCN1S(=O)(=O)Cc1ccc(F)c(NC(=O)C2CN(C(=O)CNc3cccc4c3CN([C@@H]3CCC(=O)NC3=O)C4=O)C2)c1] |
| 148b | 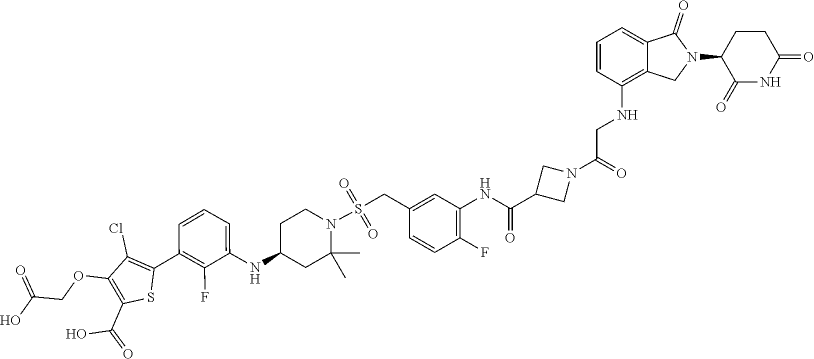 [SMILES: CC1(C)C[C@@H](Nc2cccc(-c3sc(C(=O)O)c(OCC(=O)O)c3Cl)c2F)CCN1S(=O)(=O)Cc1ccc(F)c(NC(=O)C2CN(C(=O)CNc3cccc4c3CN([C@H]3CCC(=O)NC3=O)C4=O)C2)c1] |
| 149 | 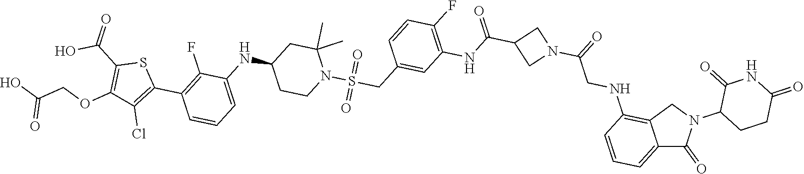 [SMILES: CC1(C)C[C@H](Nc2cccc(-c3sc(C(=O)O)c(OCC(=O)O)c3Cl)c2F)CCN1S(=O)(=O)Cc1ccc(F)c(NC(=O)C2CN(C(=O)CNc3cccc4c3CN(C3CCC(=O)NC3=O)C4=O)C2)c1] |
| 150 | 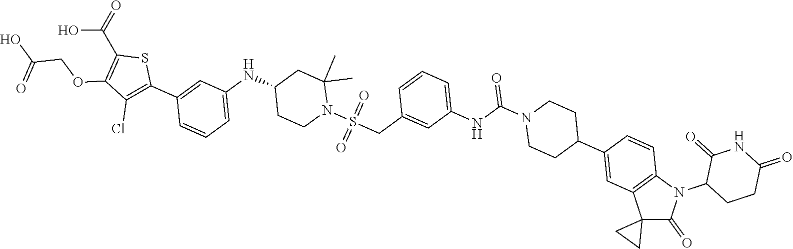 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)C3(CC3)C(=O)N4C3CCC(=O)NC3=O)CC2)c1] |
| 151 | 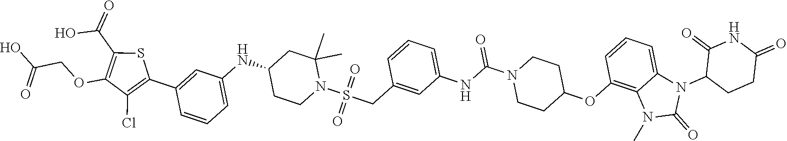 [SMILES: Cn1c(=O)n(C2CCC(=O)NC2=O)c2cccc(OC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)c21] |
| 152 | 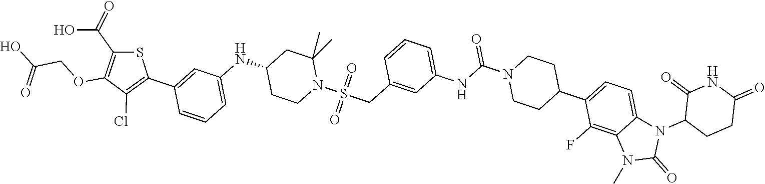 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)c(F)c21] |
| 153 | 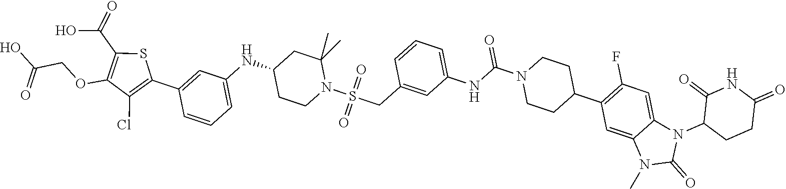 [SMILES: Cn1c(=O)n(C2CCC(=O)NC2=O)c2cc(F)c(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 154 | 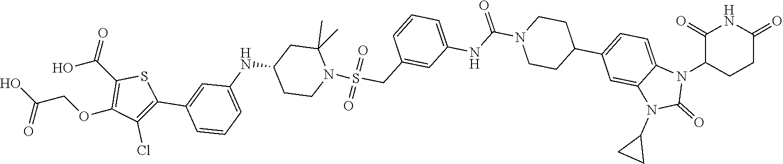 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C3CC3)c(=O)n4C3CCC(=O)NC3=O)CC2)c1] |
| 155 | 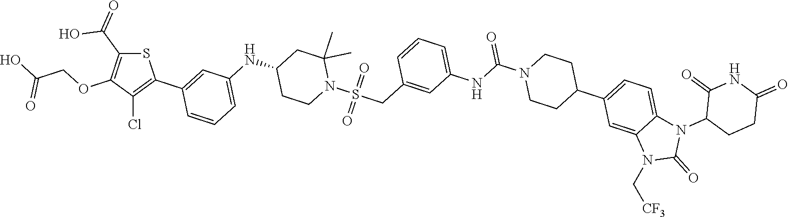 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(CC(F)(F)F)c(=O)n4C3CCC(=O)NC3=O)CC2)c1] |
| 156 | 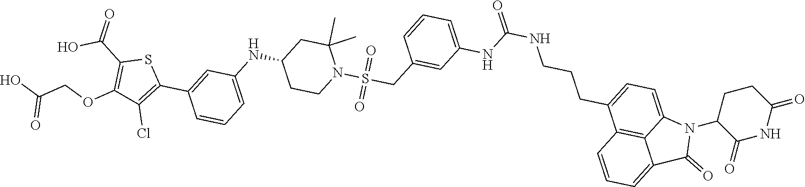 [SMILES: CC1(C)C[C@@H](Nc2cccc(-c3sc(C(=O)O)c(OCC(=O)O)c3Cl)c2)CCN1S(=O)(=O)Cc1cccc(NC(=O)NCCCc2ccc3c4c(cccc24)C(=O)N3C2CCC(=O)NC2=O)c1] |
| 157 | 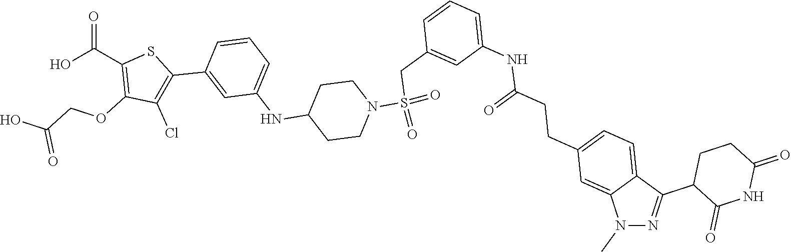 [SMILES: Cn1nc(C2CCC(=O)NC2=O)c2ccc(CCC(=O)Nc3cccc(CS(=O)(=O)N4CCC(Nc5cccc(-c6sc(C(=O)O)c(OCC(=O)O)c6Cl)c5)CC4)c3)cc21] |
| 158 | 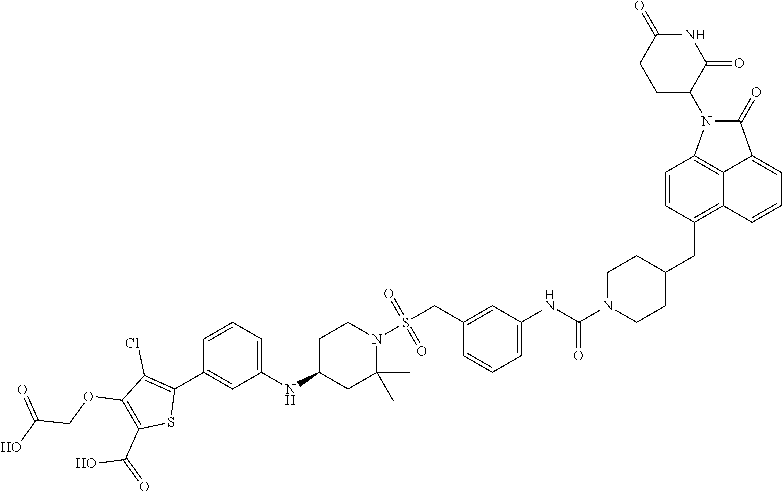 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(Cc3ccc4c5c(cccc35)C(=O)N4C3CCC(=O)NC3=O)CC2)c1] |
| 159 | 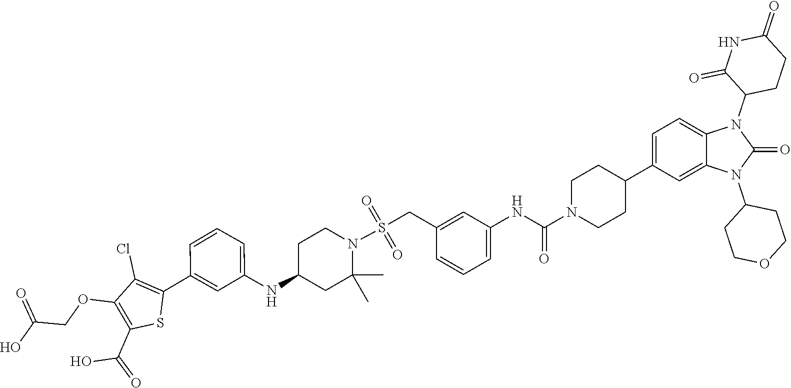 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C3CCOCC3)c(=O)n4C3CCC(=O)NC3=O)CC2)c1] |
| 160 | 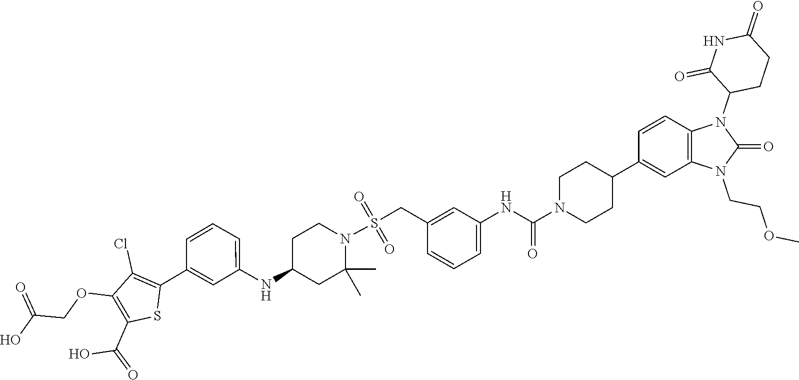 [SMILES: COCCn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 161 | 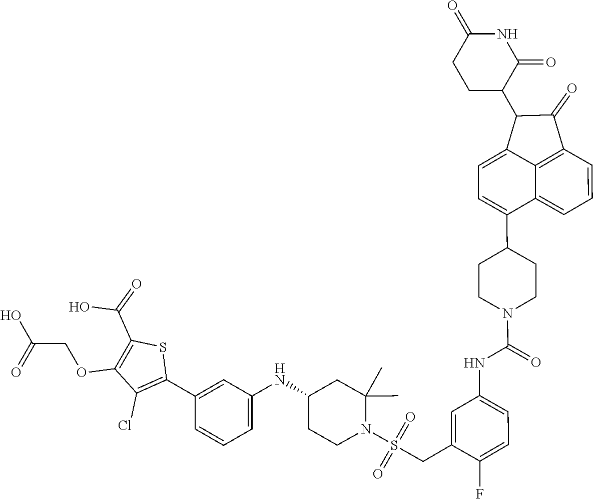 [SMILES: CC1(C)C[C@@H](Nc2cccc(-c3sc(C(=O)O)c(OCC(=O)O)c3Cl)c2)CCN1S(=O)(=O)Cc1cc(NC(=O)N2CCC(c3ccc4c5c(cccc35)C(=O)C4C3CCC(=O)NC3=O)CC2)ccc1F] |
| 162 | 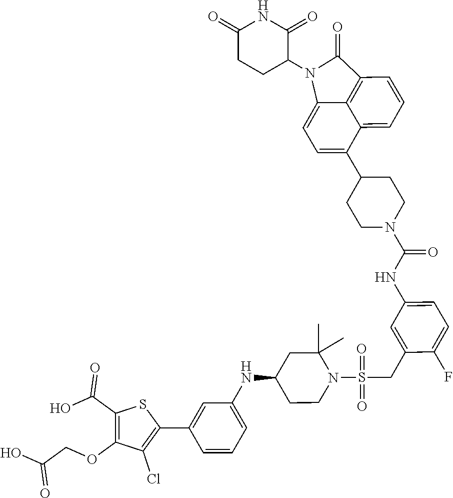 [SMILES: CC1(C)C[C@H](Nc2cccc(-c3sc(C(=O)O)c(OCC(=O)O)c3Cl)c2)CCN1S(=O)(=O)Cc1cc(NC(=O)N2CCC(c3ccc4c5c(cccc35)C(=O)N4C3CCC(=O)NC3=O)CC2)ccc1F] |
| 163 | 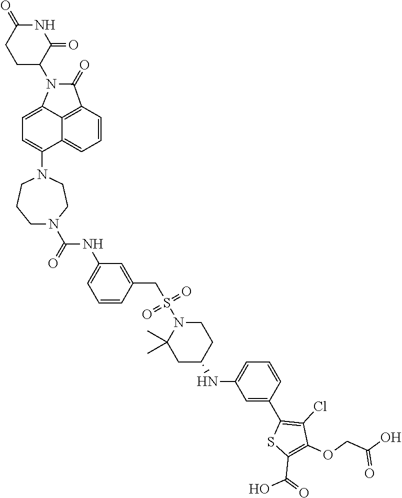 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCCN(c3ccc4c5c(cccc35)C(=O)N4C3CCC(=O)NC3=O)CC2)c1] |
| 164 | 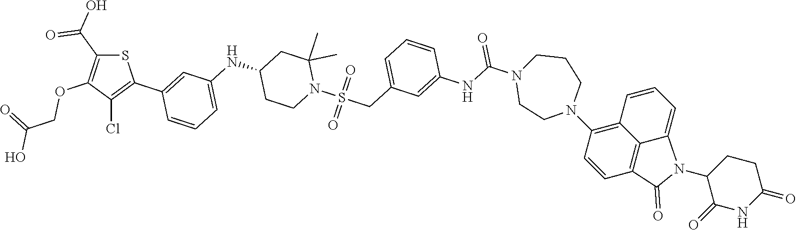 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCCN(c3ccc4c5c(cccc35)N(C3CCC(=O)NC3=O)C4=O)CC2)c1] |
| 165 | 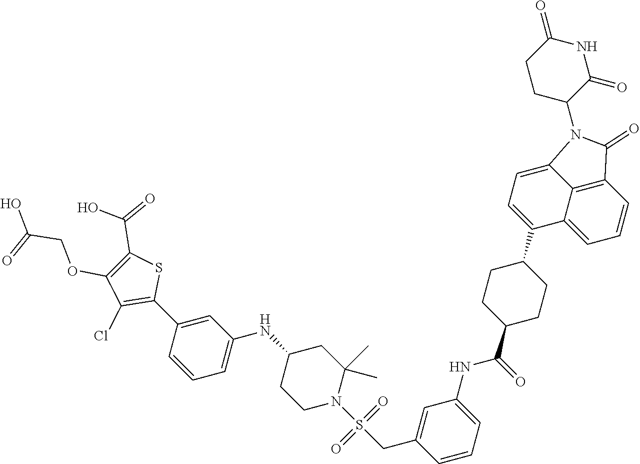 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@H](c3ccc4c5c(cccc53)C(=O)N4C3CCC(=O)NC3=O)CC2)c1] |
| 166 | 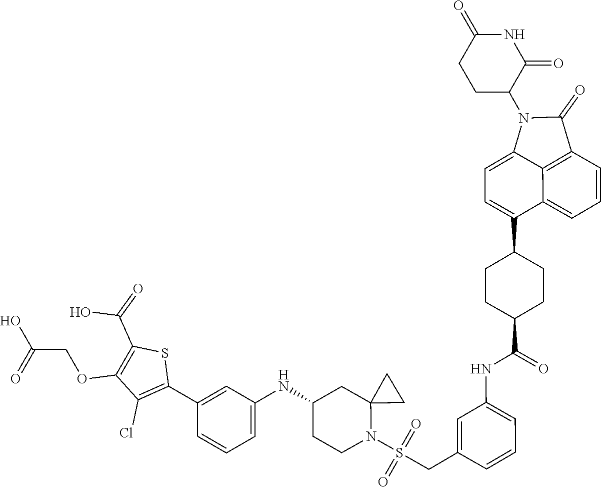 [SMILES: O=C(O)COc1c(C(=O)O)sc(-c2cccc(N[C@H]3CCN(S(=O)(=O)Cc4cccc(NC(=O)[C@H]5CC[C@@H](c6ccc7c8c(cccc86)C(=O)N7C6CCC(=O)NC6=O)CC5)c4)C4(CC4)C3)c2)c1Cl] |
| 167 | 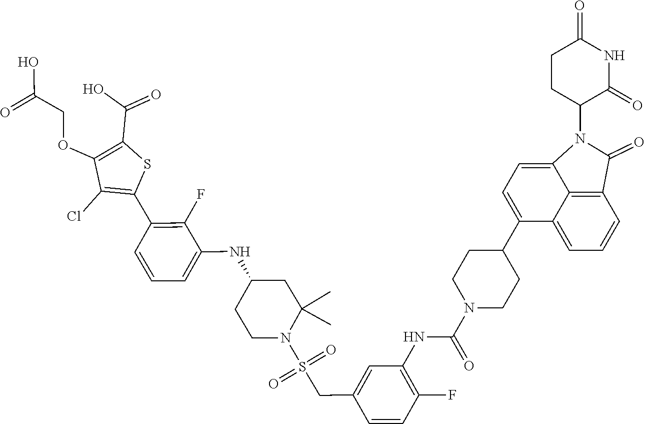 [SMILES: CC1(C)C[C@@H](Nc2cccc(-c3sc(C(=O)O)c(OCC(=O)O)c3Cl)c2F)CCN1S(=O)(=O)Cc1ccc(F)c(NC(=O)N2CCC(c3ccc4c5c(cccc35)C(=O)N4C3CCC(=O)NC3=O)CC2)c1] |
| 168 | 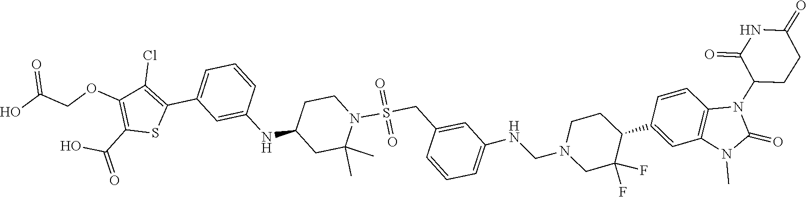 [SMILES: Cn1c(=O)n(C2CCC(=O)NC2=O)c2ccc([C@H]3CCN(CNc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 169 | 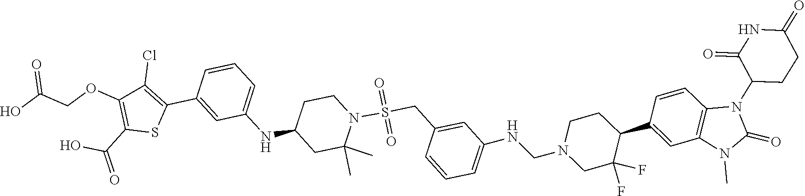 [SMILES: Cn1c(=O)n(C2CCC(=O)NC2=O)c2ccc([C@@H]3CCN(CNc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 169a | 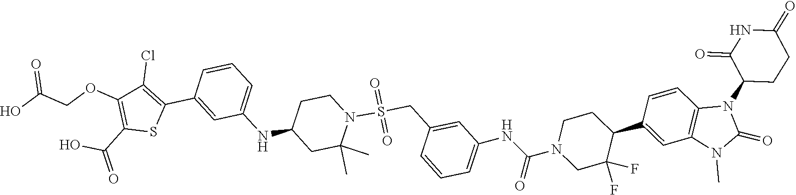 [SMILES: Cn1c(=O)n([C@@H]2CCC(=O)NC2=O)c2ccc([C@@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 169b | 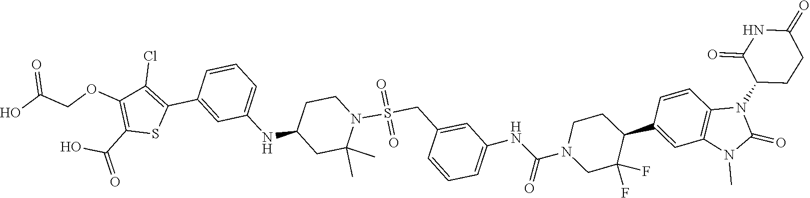 [SMILES: Cn1c(=O)n([C@H]2CCC(=O)NC2=O)c2ccc([C@@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 170 | 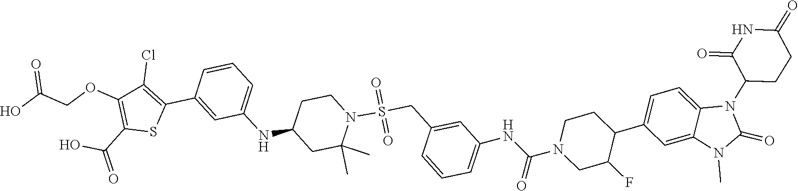 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3F)cc21] |
| 171 | 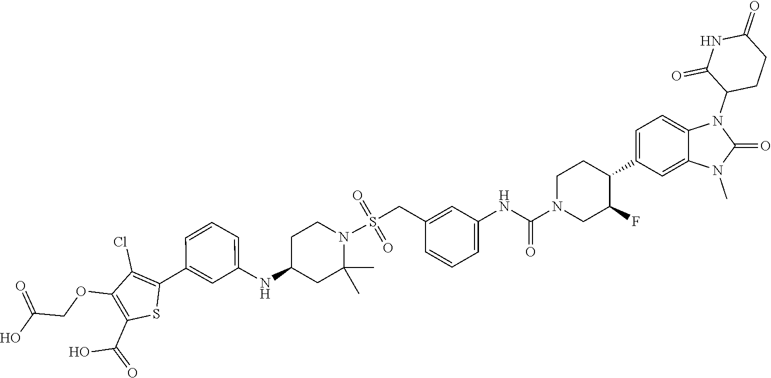 [SMILES: Cn1c(=O)n(C2CCC(=O)NC2=O)c2ccc([C@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)C[C@@H]3F)cc21] |
| 172 | 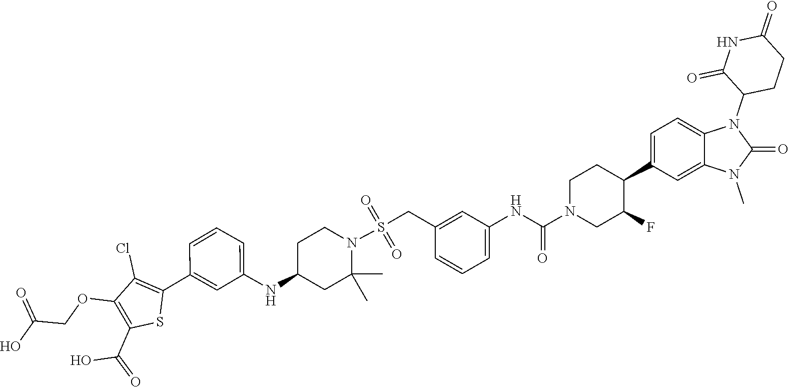 [SMILES: Cn1c(=O)n(C2CCC(=O)NC2=O)c2ccc([C@@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)C[C@@H]3F)cc21] |
| 173 | 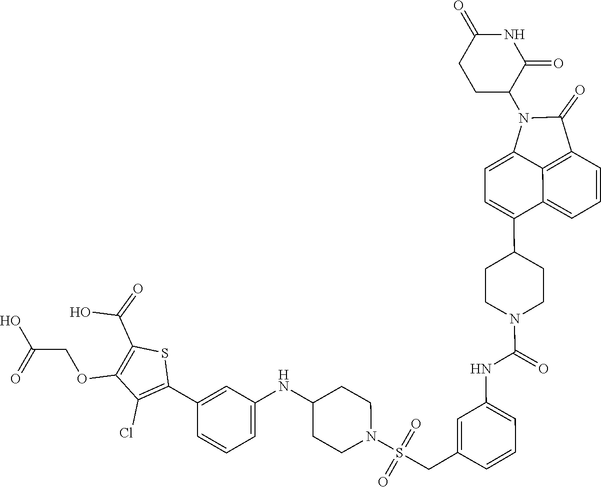 [SMILES: O=C(O)COc1c(C(=O)O)sc(-c2cccc(NC3CCN(S(=O)(=O)Cc4cccc(NC(=O)N5CCC(c6ccc7c8c(cccc68)C(=O)N7C6CCC(=O)NC6=O)CC5)c4)CC3)c2)c1Cl] |
| 174 | 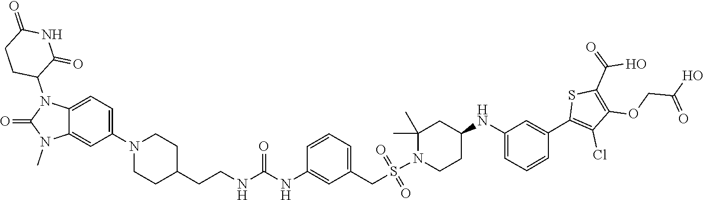 [SMILES: Cn1c(=O)n(C2CCC(=O)NC2=O)c2ccc(N3CCC(CCNC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 175 | 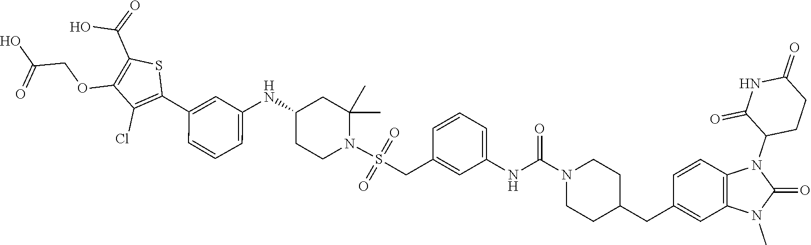 [SMILES: Cn1c(=O)n(C2CCC(=O)NC2=O)c2ccc(CC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 176 | 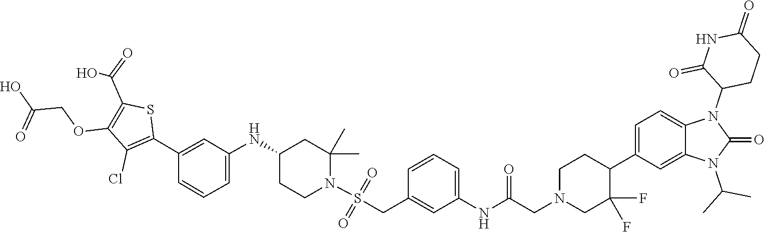 [SMILES: CC(C)n1c(=O)n(C2CCC(=O)NC2=O)c2ccc(C3CCN(CC(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 177 | 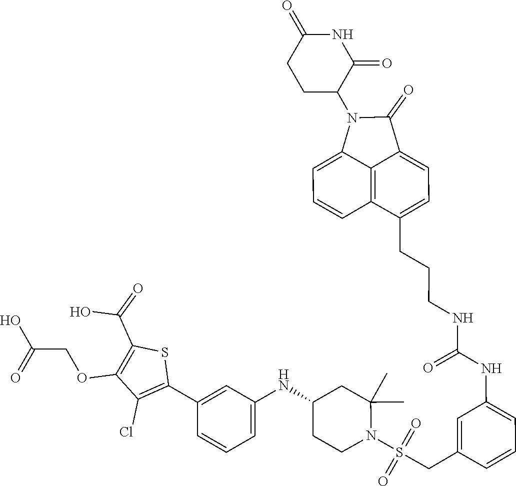 [SMILES: CC1(C)C[C@@H](Nc2cccc(-c3sc(C(=O)O)c(OCC(=O)O)c3Cl)c2)CCN1S(=O)(=O)Cc1cccc(NC(=O)NCCCc2ccc3c4c(cccc24)N(C2CCC(=O)NC2=O)C3=O)c1] |
| 178 | 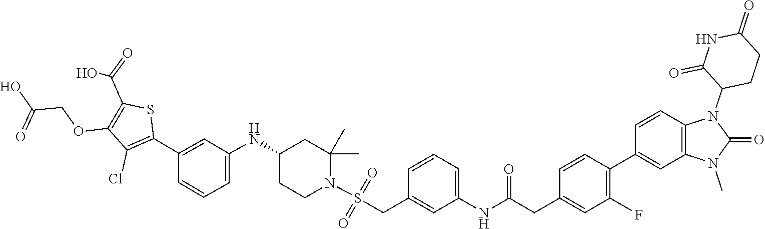 [SMILES: Cn1c(=O)n(C2CCC(=O)NC2=O)c2ccc(-c3ccc(CC(=O)Nc4cccc(CS(=O)(=O)N5CC[C@H](Nc6cccc(-c7sc(C(=O)O)c(OCC(=O)O)c7Cl)c6)CC5(C)C)c4)cc3F)cc21] |
| 179 | 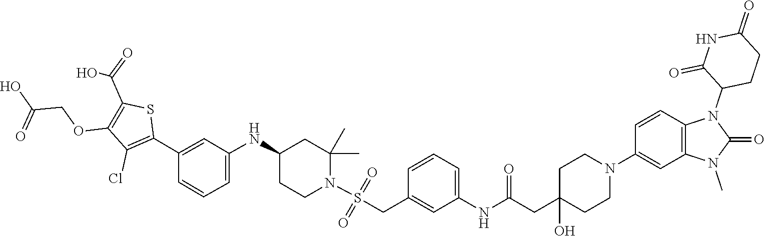 [SMILES: Cn1c(=O)n(C2CCC(=O)NC2=O)c2ccc(N3CCC(O)(CC(=O)Nc4cccc(CS(=O)(=O)N5CC[C@@H](Nc6cccc(-c7sc(C(=O)O)c(OCC(=O)O)c7Cl)c6)CC5(C)C)c4)CC3)cc21] |
| 180 | 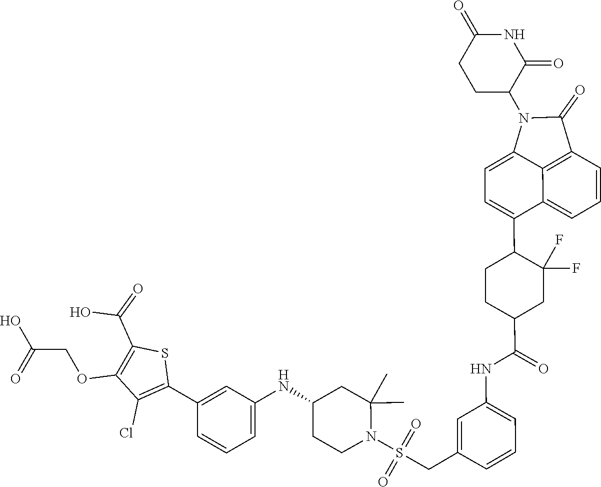 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCC(c3ccc4c5c(cccc35)C(=O)N4C3CCC(=O)NC3=O)C(F)(F)C2)c1] |
| 181 | 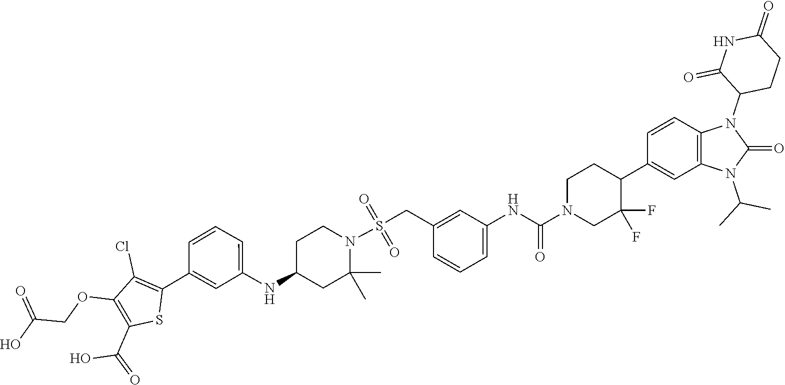 [SMILES: CC(C)n1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 182 | 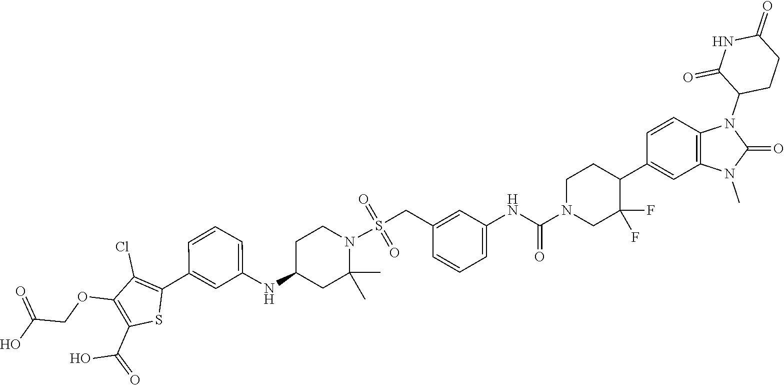 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 183 | 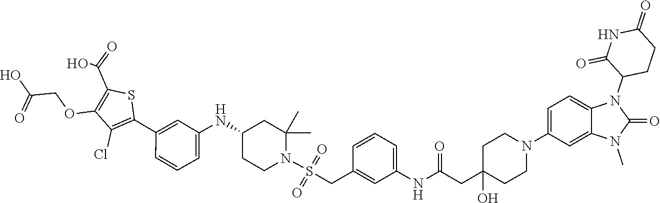 [SMILES: Cn1c(=O)n(C2CCC(=O)NC2=O)c2ccc(N3CCC(O)(CC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 184 | 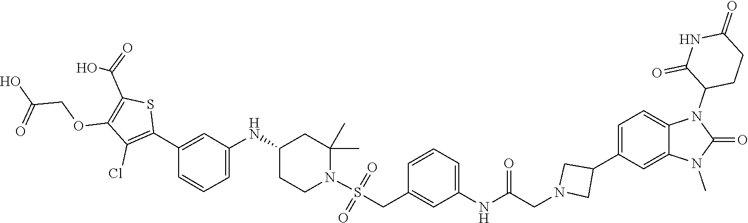 [SMILES: Cn1c(=O)n(C2CCC(=O)NC2=O)c2ccc(C3CN(CC(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 185 | 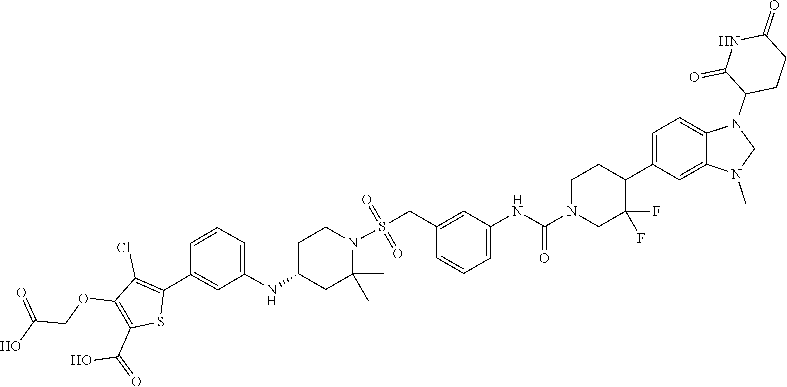 [SMILES: CN1CN(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6)CC5(C)C)c4)CC3(F)F)cc21] |
| 186 | 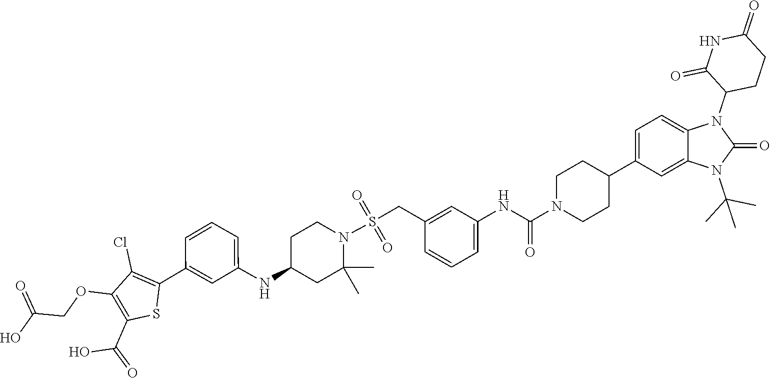 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C(C)(C)C)c(=O)n4C3CCC(=O)NC3=O)CC2)c1] |
| 187a | 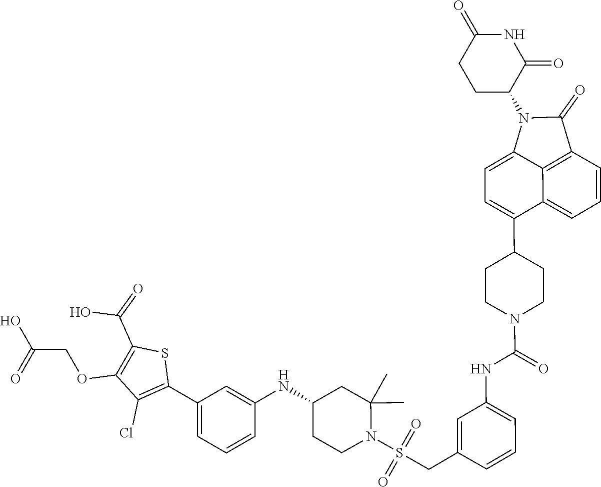 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4[C@@H]3CCC(=O)NC3=O)CC2)c1] |
| 187b | 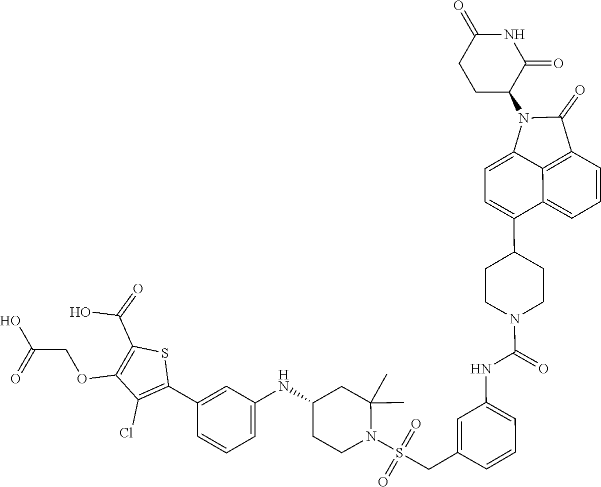 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4[C@H]3CCC(=O)NC3=O)CC2)c1] |
| 188 | 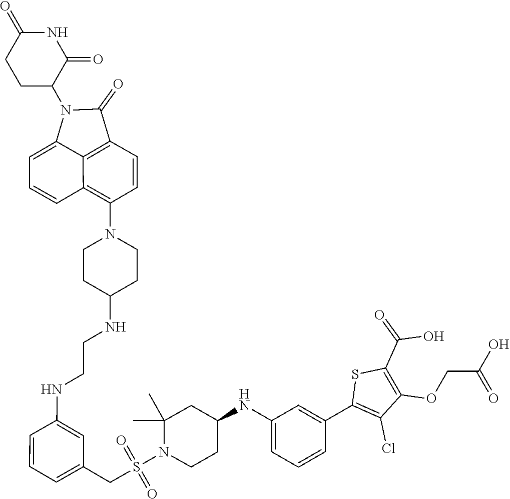 [SMILES: CC1(C)C[C@@H](Nc2cccc(-c3sc(C(=O)O)c(OCC(=O)O)c3Cl)c2)CCN1S(=O)(=O)Cc1cccc(NCCNC2CCN(c3ccc4c5c(cccc35)N(C3CCC(=O)NC3=O)C4=O)CC2)c1] |
| 189 | 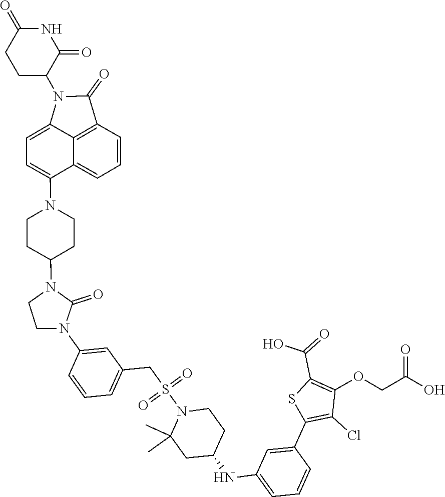 [SMILES: CC1(C)C[C@@H](Nc2cccc(-c3sc(C(=O)O)c(OCC(=O)O)c3Cl)c2)CCN1S(=O)(=O)Cc1cccc(N2CCN(C3CCN(c4ccc5c6c(cccc46)C(=O)N5C4CCC(=O)NC4=O)CC3)C2=O)c1] |
| 190 | 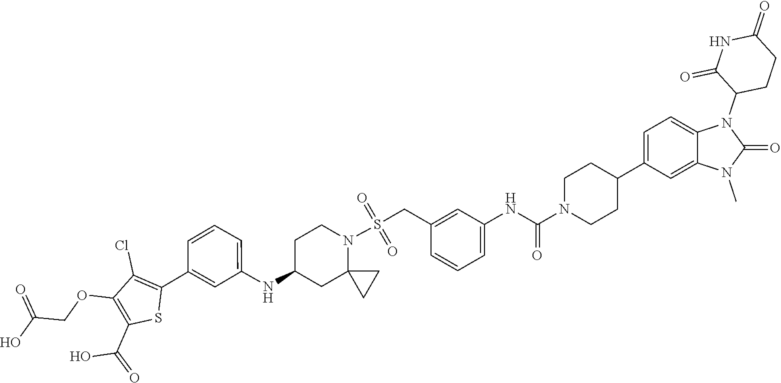 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC56CC6)c4)CC3)cc21] |
| 191 | 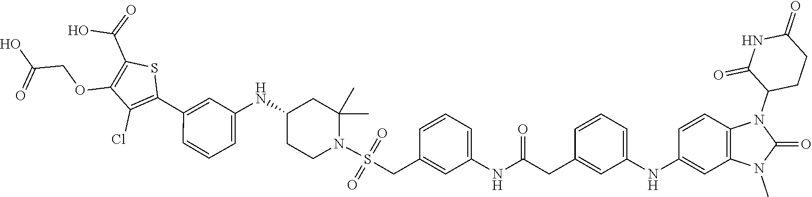 [SMILES: Cn1c(=O)n(C2CCC(=O)NC2=O)c2ccc(Nc3cccc(CC(=O)Nc4cccc(CS(=O)(=O)N5CC[C@H](Nc6cccc(-c7sc(C(=O)O)c(OCC(=O)O)c7Cl)c6)CC5(C)C)c4)c3)cc21] |
| 192 | 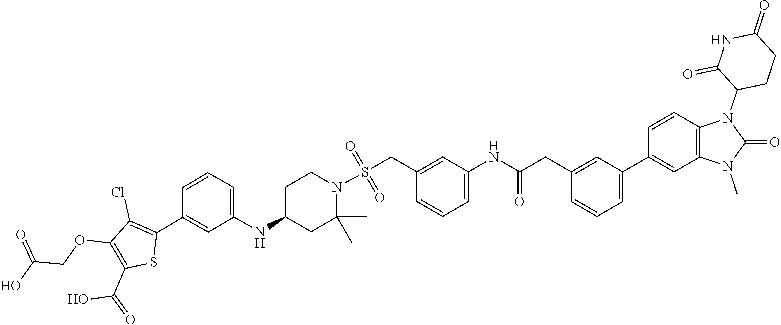 [SMILES: Cn1c(=O)n(C2CCC(=O)NC2=O)c2ccc(-c3cccc(CC(=O)Nc4cccc(CS(=O)(=O)N5CC[C@H](Nc6cccc(-c7sc(C(=O)O)c(OCC(=O)O)c7Cl)c6)CC5(C)C)c4)c3)cc21] |
| 193 | 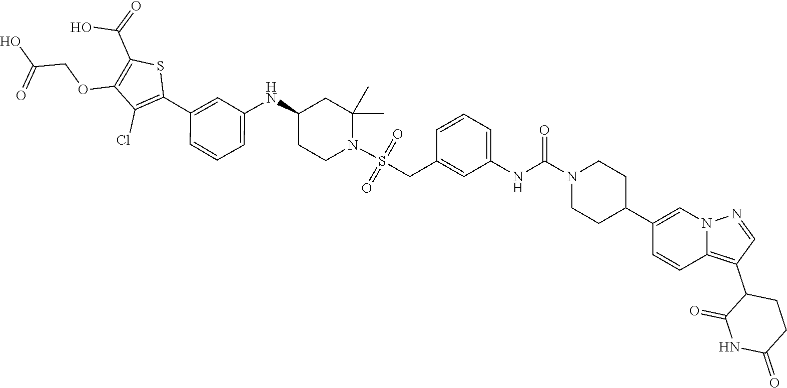 [SMILES: CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c(C5CCC(=O)NC5=O)cnn4c3)CC2)c1] |
| 194 | 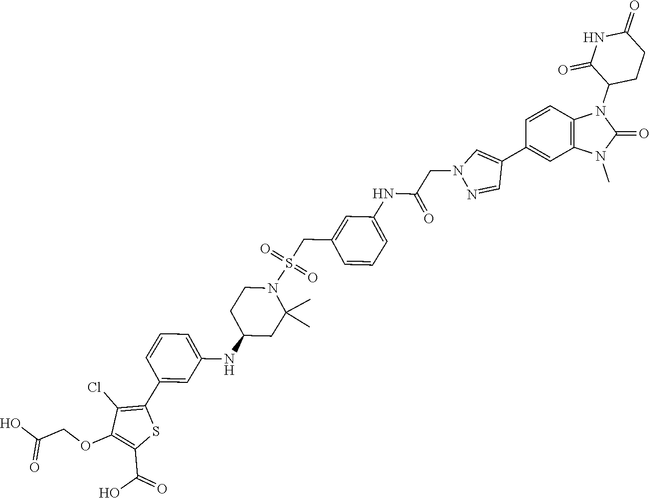 [SMILES: Cn1c(=O)n(C2CCC(=O)NC2=O)c2ccc(-c3cnn(CC(=O)Nc4cccc(CS(=O)(=O)N5CC[C@H](Nc6cccc(-c7sc(C(=O)O)c(OCC(=O)O)c7Cl)c6)CC5(C)C)c4)c3)cc21] |
| 195 | 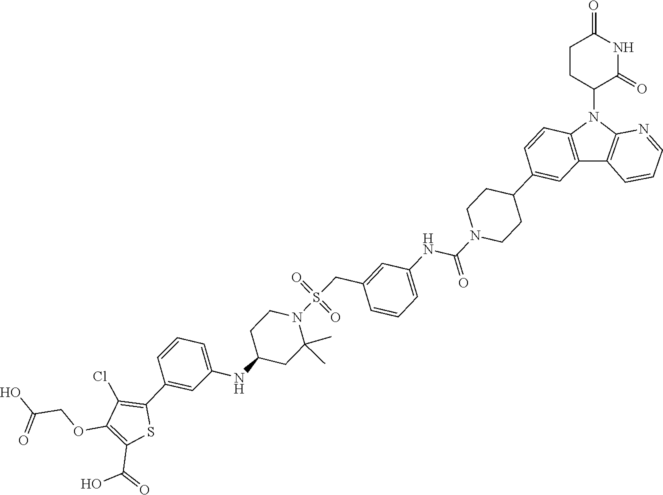 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)c3cccnc3n4C3CCC(=O)NC3=O)CC2)c1] |
| 196 | 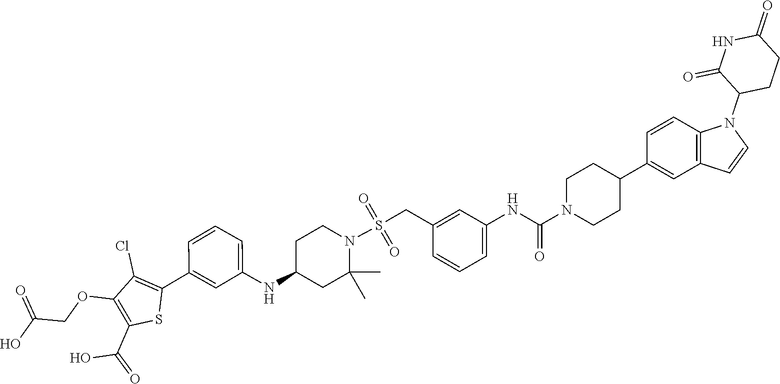 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(ccn4C4CCC(=O)NC4=O)c3)CC2)c1] |
| 197 | 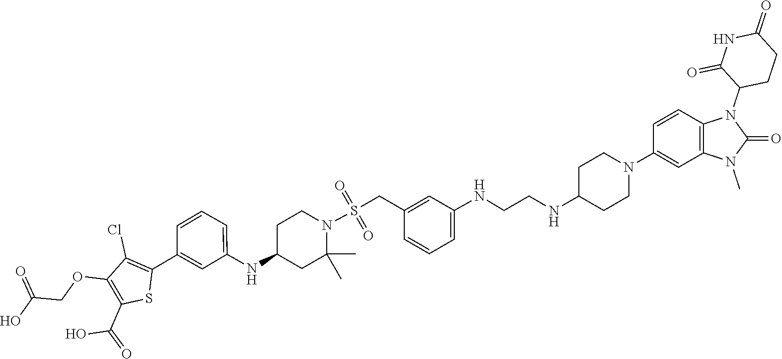 [SMILES: Cn1c(=O)n(C2CCC(=O)NC2=O)c2ccc(N3CCC(NCCNc4cccc(CS(=O)(=O)N5CC[C@H](Nc6cccc(-c7sc(C(=O)O)c(OCC(=O)O)c7Cl)c6)CC5(C)C)c4)CC3)cc21] |
| 198 | 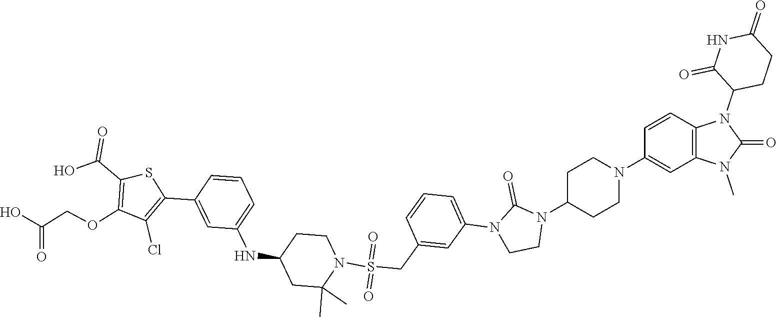 [SMILES: Cn1c(=O)n(C2CCC(=O)NC2=O)c2ccc(N3CCC(N4CCN(c5cccc(CS(=O)(=O)N6CC[C@H](Nc7cccc(-c8sc(C(=O)O)c(OCC(=O)O)c8Cl)c7)CC6(C)C)c5)C4=O)CC3)cc21] |
| 200 | 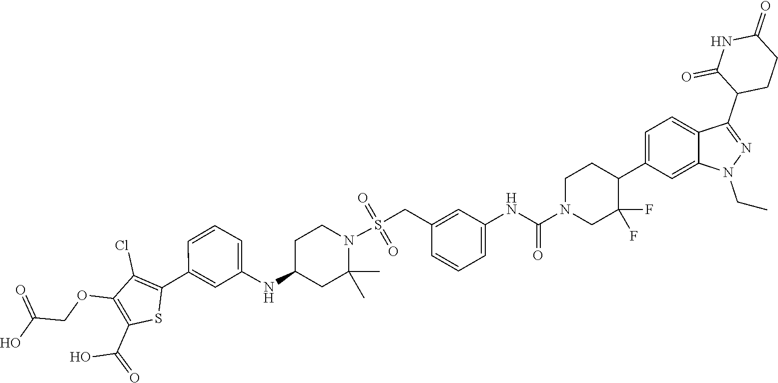 [SMILES: CCn1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] |
| 201 | 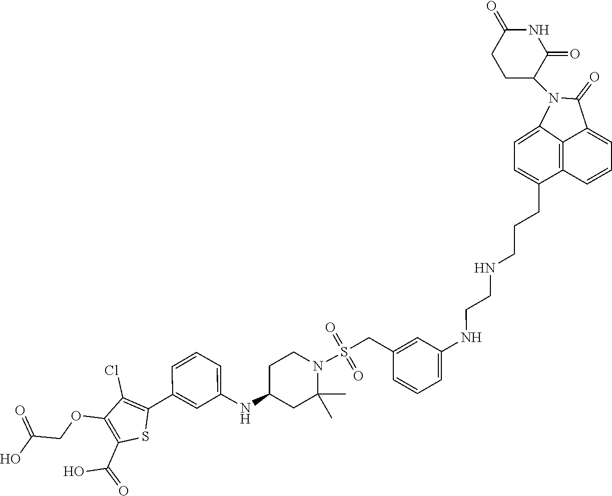 [SMILES: CC1(C)C[C@@H](Nc2cccc(-c3sc(C(=O)O)c(OCC(=O)O)c3Cl)c2)CCN1S(=O)(=O)Cc1cccc(NCCNCCCc2ccc3c4c(cccc24)C(=O)N3C2CCC(=O)NC2=O)c1] |
| 202 | 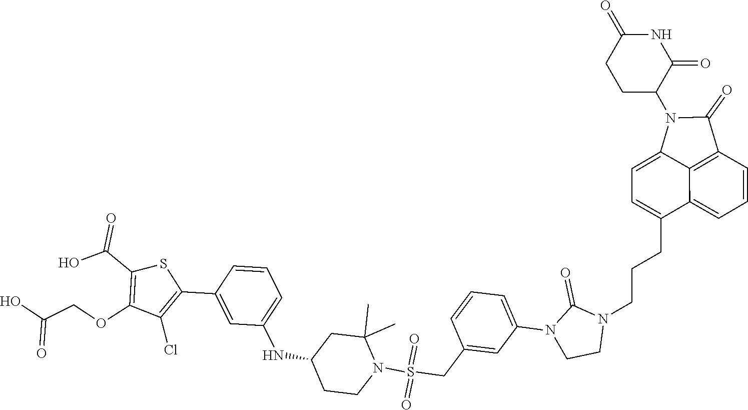 [SMILES: CC1(C)C[C@@H](Nc2cccc(-c3sc(C(=O)O)c(OCC(=O)O)c3Cl)c2)CCN1S(=O)(=O)Cc1cccc(N2CCN(CCCc3ccc4c5c(cccc35)C(=O)N4C3CCC(=O)NC3=O)C2=O)c1] |
| 203 | 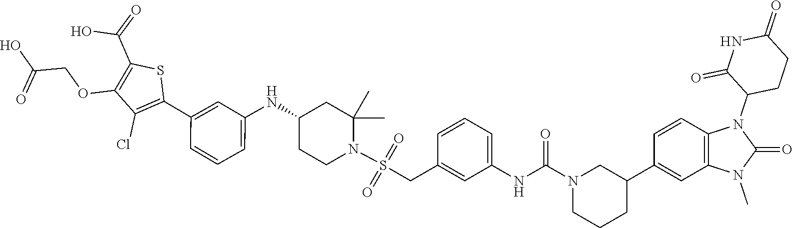 [SMILES: Cn1c(=O)n(C2CCC(=O)NC2=O)c2ccc(C3CCCN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21] |
| 204 | 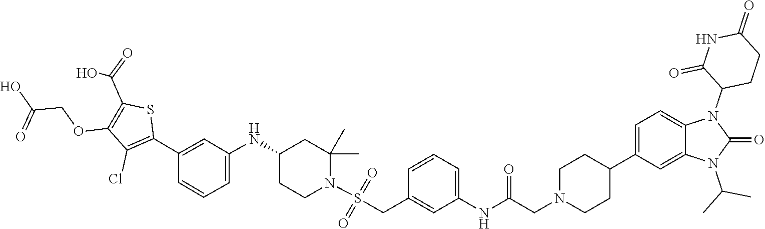 [SMILES: CC(C)n1c(=O)n(C2CCC(=O)NC2=O)c2ccc(C3CCN(CC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 205 | 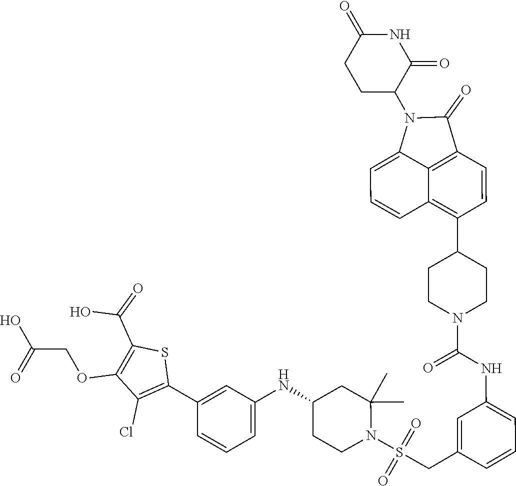 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)N(C3CCC(=O)NC3=O)C4=O)CC2)c1] |
| 206 | 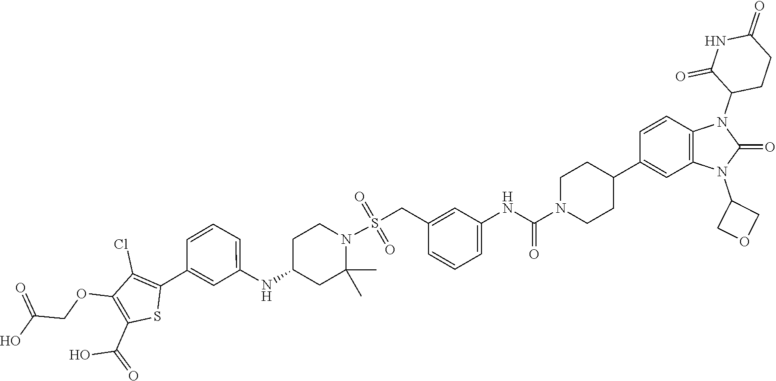 [SMILES: CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C3COC3)c(=O)n4C3CCC(=O)NC3=O)CC2)c1] |
| 207 | 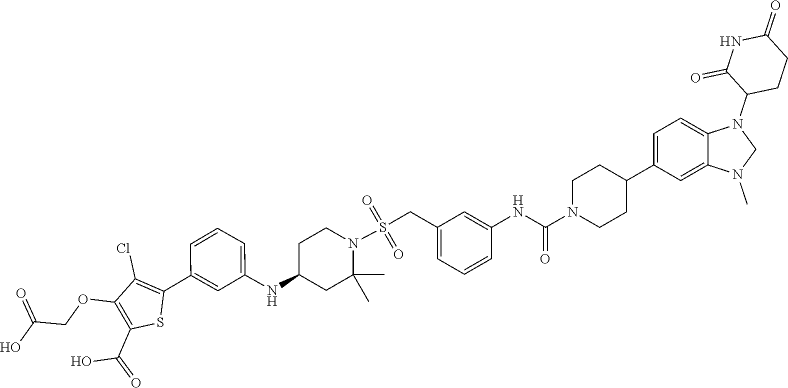 [SMILES: CN1CN(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 208 | 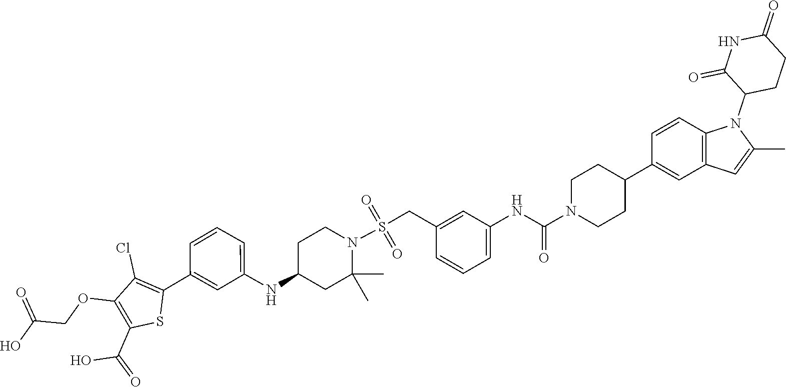 [SMILES: Cc1cc2cc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)ccc2n1C1CCC(=O)NC1=O] |
| 209 | 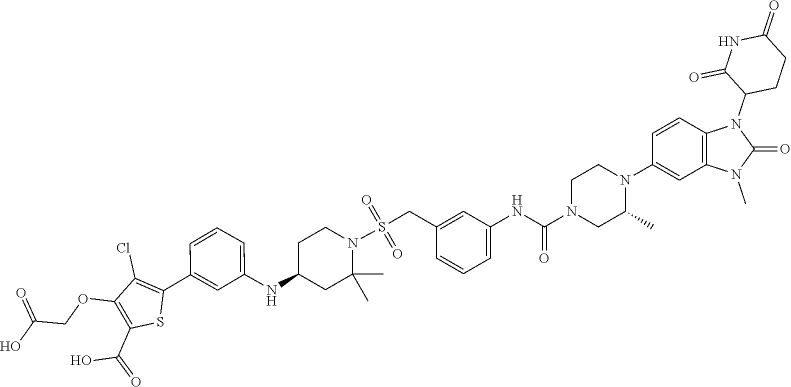 [SMILES: C[C@@H]1CN(C(=O)Nc2cccc(CS(=O)(=O)N3CC[C@H](Nc4cccc(-c5sc(C(=O)O)c(OCC(=O)O)c5Cl)c4)CC3(C)C)c2)CCN1c1ccc2c(c1)n(C)c(=O)n2C1CCC(=O)NC1=O] |
| 210 | 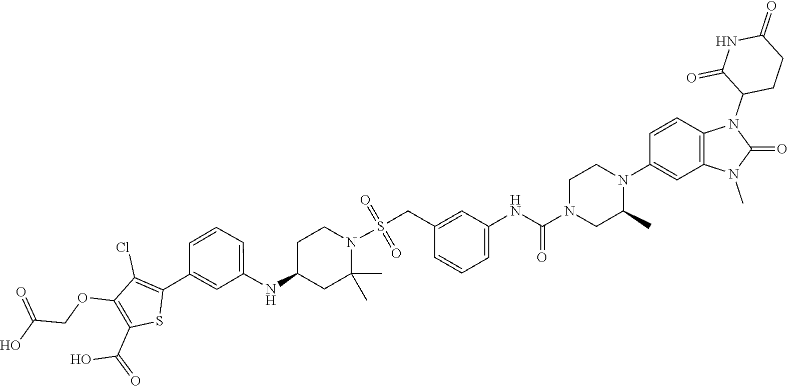 [SMILES: C[C@H]1CN(C(=O)Nc2cccc(CS(=O)(=O)N3CC[C@H](Nc4cccc(-c5sc(C(=O)O)c(OCC(=O)O)c5Cl)c4)CC3(C)C)c2)CCN1c1ccc2c(c1)n(C)c(=O)n2C1CCC(=O)NC1=O] |
| 211 | 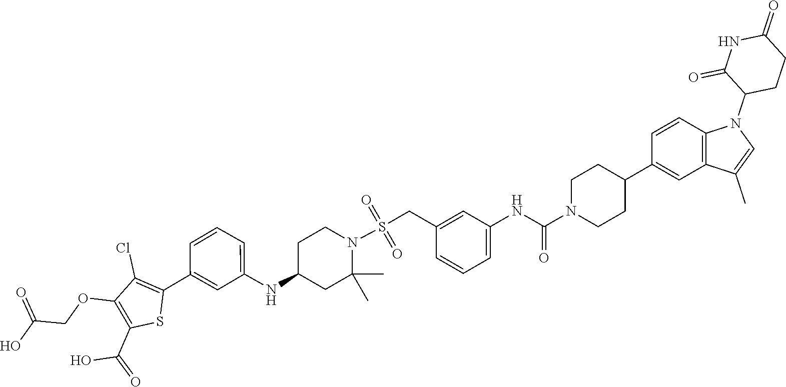 [SMILES: Cc1cn(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc12] |
| 212 | 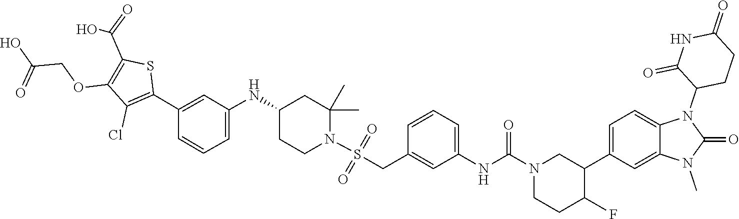 [SMILES: Cn1c(=O)n(C2CCC(=O)NC2=O)c2ccc(C3CN(C(=O)Nc4cccc(CS(=O)(=O)N5CC[C@H](Nc6cccc(-c7sc(C(=O)O)c(OCC(=O)O)c7Cl)c6)CC5(C)C)c4)CCC3F)cc21] |
| 213 | 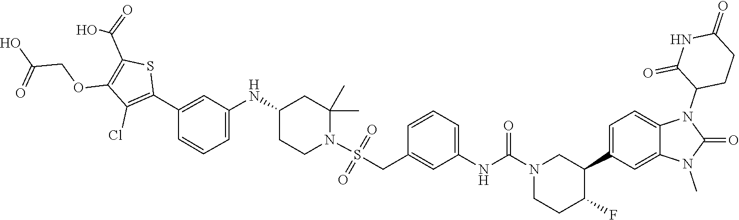 [SMILES: Cn1c(=O)n(C2CCC(=O)NC2=O)c2ccc([C@@H]3CN(C(=O)Nc4cccc(CS(=O)(=O)N5CC[C@H](Nc6cccc(-c7sc(C(=O)O)c(OCC(=O)O)c7Cl)c6)CC5(C)C)c4)CC[C@H]3F)cc21] |
| 214 | 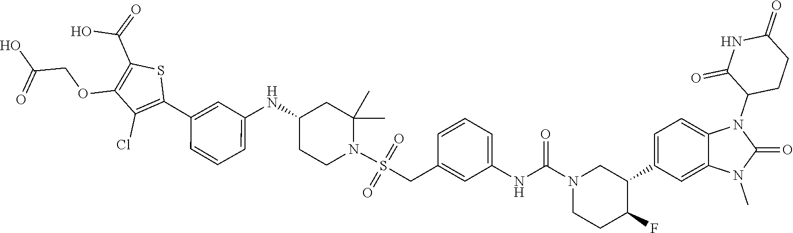 [SMILES: Cn1c(=O)n(C2CCC(=O)NC2=O)c2ccc([C@H]3CN(C(=O)Nc4cccc(CS(=O)(=O)N5CC[C@H](Nc6cccc(-c7sc(C(=O)O)c(OCC(=O)O)c7Cl)c6)CC5(C)C)c4)CC[C@@H]3F)cc21] |
| 215 | 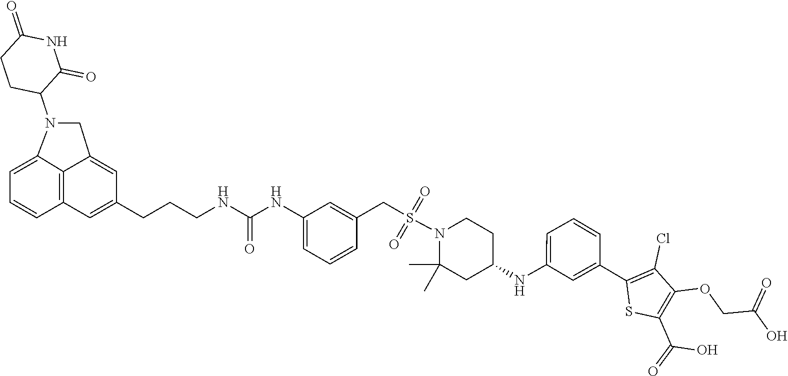 [SMILES: CC1(C)C[C@@H](Nc2cccc(-c3sc(C(=O)O)c(OCC(=O)O)c3Cl)c2)CCN1S(=O)(=O)Cc1cccc(NC(=O)NCCCc2cc3c4c(cccc4c2)N(C2CCC(=O)NC2=O)C3)c1] |
| 216 | 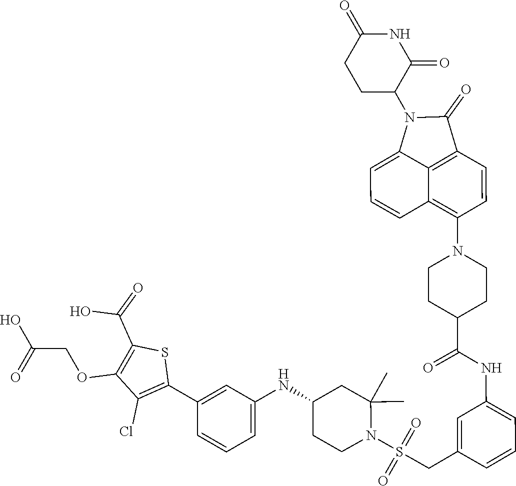 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(c3ccc4c5c(cccc35)N(C3CCC(=O)NC3=O)C4=O)CC2)c1] |
| 217 | 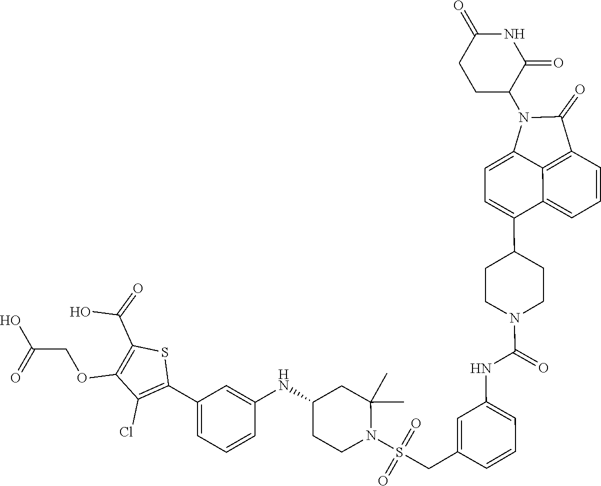 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1] |
| 218 | 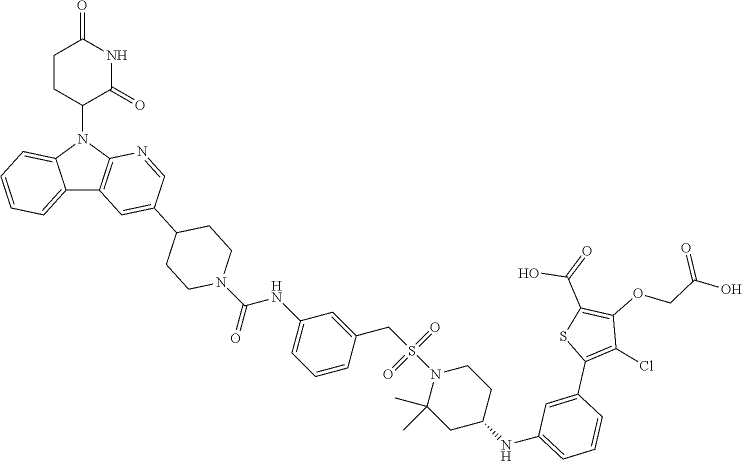 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3cnc4c(c3)c3ccccc3n4C3CCC(=O)NC3=O)CC2)c1] |
| 219 | 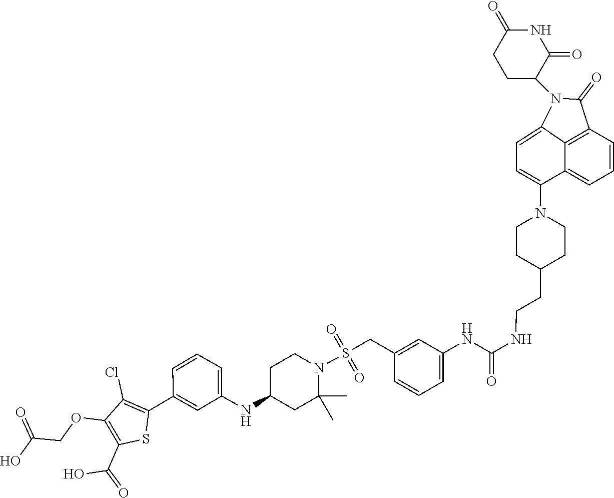 [SMILES: CC1(C)C[C@@H](Nc2cccc(-c3sc(C(=O)O)c(OCC(=O)O)c3Cl)c2)CCN1S(=O)(=O)Cc1cccc(NC(=O)NCCC2CCN(c3ccc4c5c(cccc35)C(=O)N4C3CCC(=O)NC3=O)CC2)c1] |
| 220 | 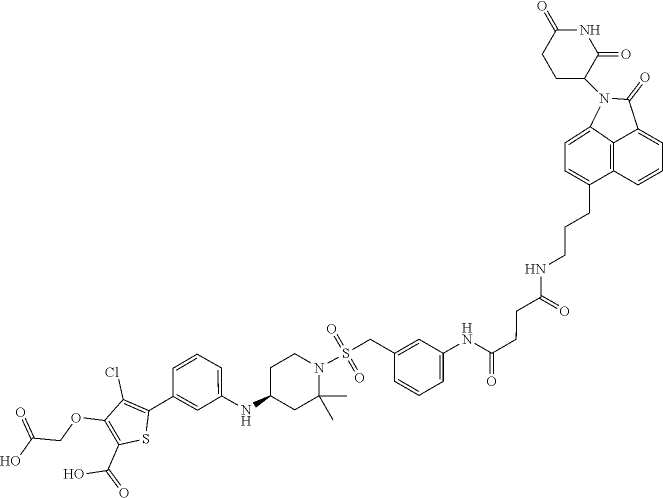 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCC(=O)NCCCc2ccc3c4c(cccc24)C(=O)N3C2CCC(=O)NC2=O)c1] |
| 221 | 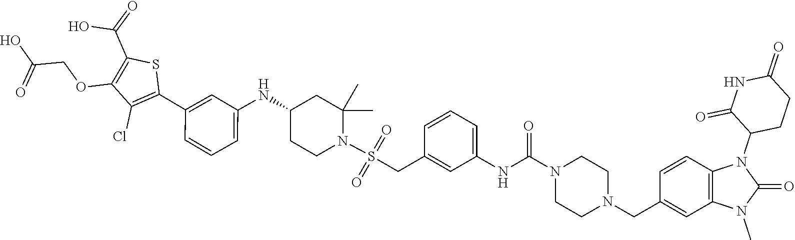 [SMILES: Cn1c(=O)n(C2CCC(=O)NC2=O)c2ccc(CN3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 222 | 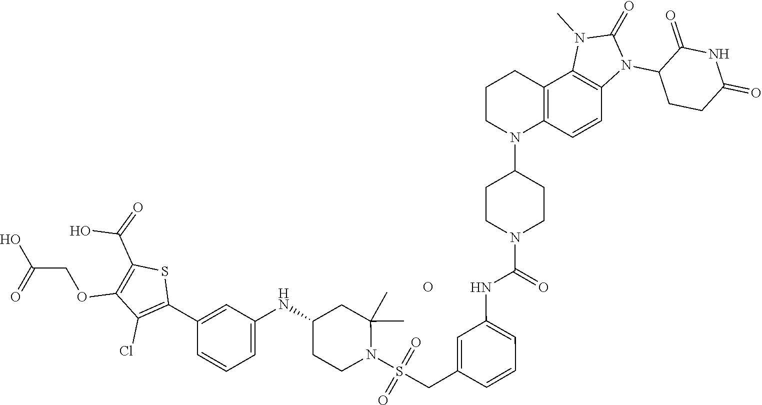 |
| 223 | 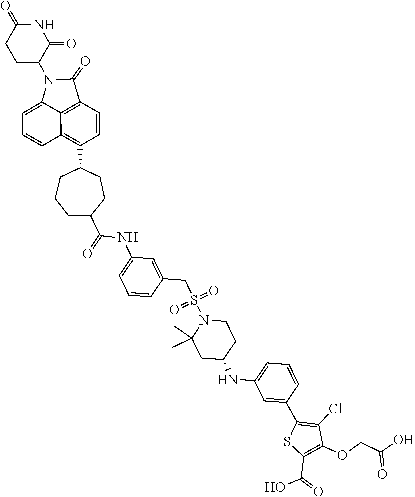 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCC[C@H](c3ccc4c5c(cccc35)N(C3CCC(=O)NC3=O)C4=O)CC2)c1] |
| 224 | 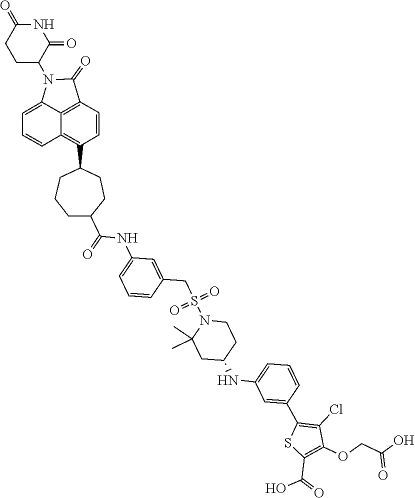 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCC[C@@H](c3ccc4c5c(cccc35)N(C3CCC(=O)NC3=O)C4=O)CC2)c1] |
| 225 | 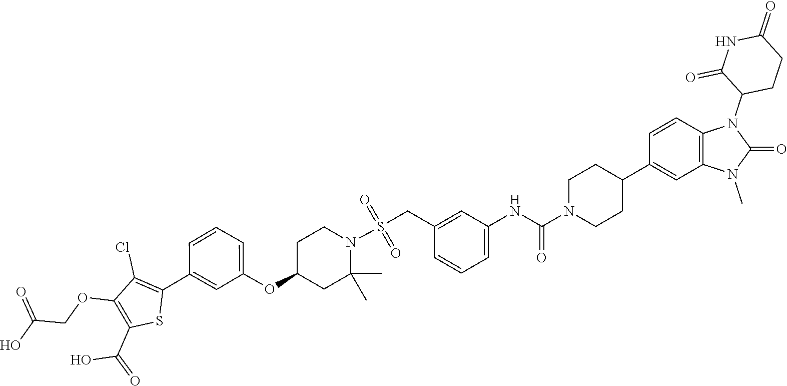 [SMILES: Cn1c(=O)n(C2CCC(=O)NC2=O)c2ccc(C3CCN(C(=O)Nc4cccc(CS(=O)(=O)N5CC[C@H](Oc6cccc(-c7sc(C(=O)O)c(OCC(=O)O)c7Cl)c6)CC5(C)C)c4)CC3)cc21] |
| 226 | 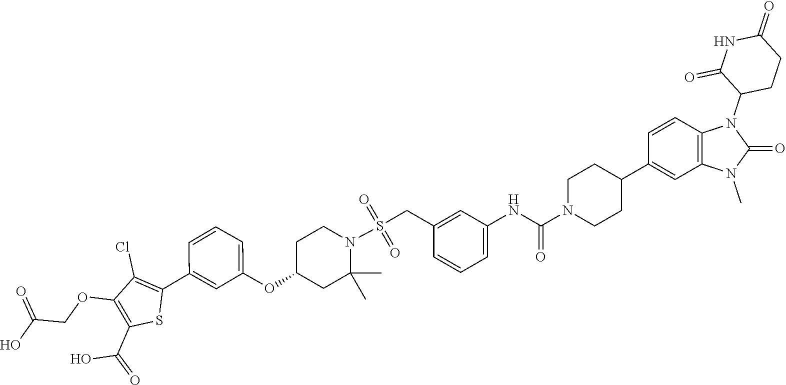 [SMILES: Cn1c(=O)n(C2CCC(=O)NC2=O)c2ccc(C3CCN(C(=O)Nc4cccc(CS(=O)(=O)N5CC[C@@H](Oc6cccc(-c7sc(C(=O)O)c(OCC(=O)O)c7Cl)c6)CC5(C)C)c4)CC3)cc21] |
| 227 | 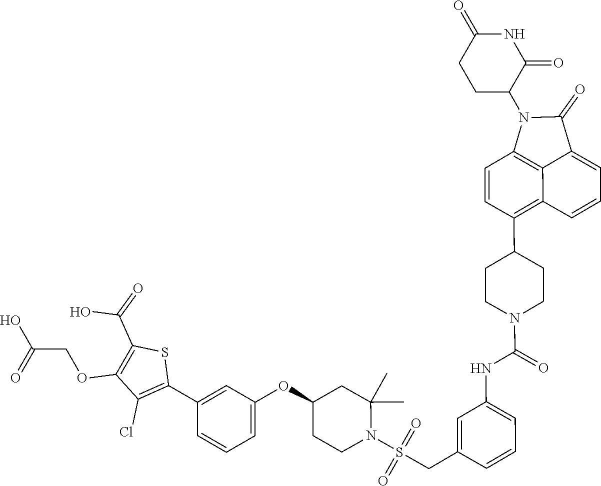 [SMILES: CC1(C)C[C@H](Oc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1] |
| 228 | 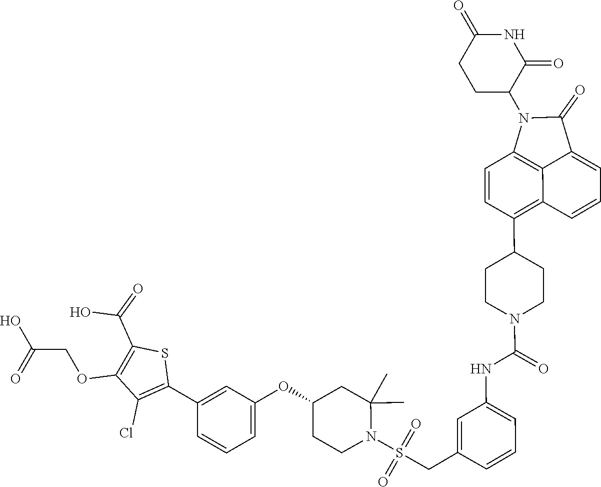 [SMILES: CC1(C)C[C@@H](Oc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1] |
| 229 | 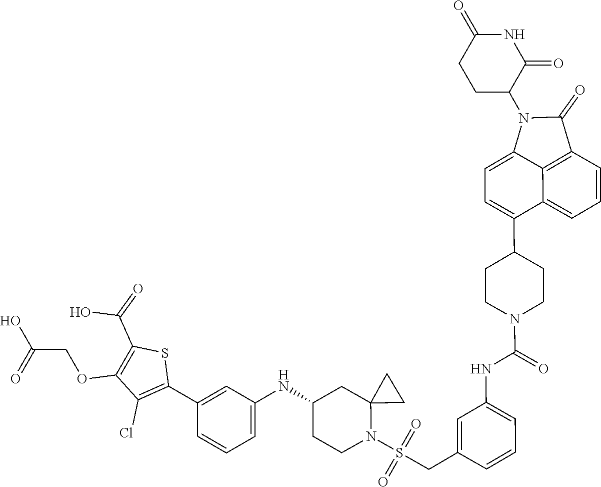 [SMILES: O=C(O)COc1c(C(=O)O)sc(-c2cccc(N[C@H]3CCN(S(=O)(=O)Cc4cccc(NC(=O)N5CCC(c6ccc7c8c(cccc68)C(=O)N7C6CCC(=O)NC6=O)CC5)c4)C4(CC4)C3)c2)c1Cl] |
| 230 | 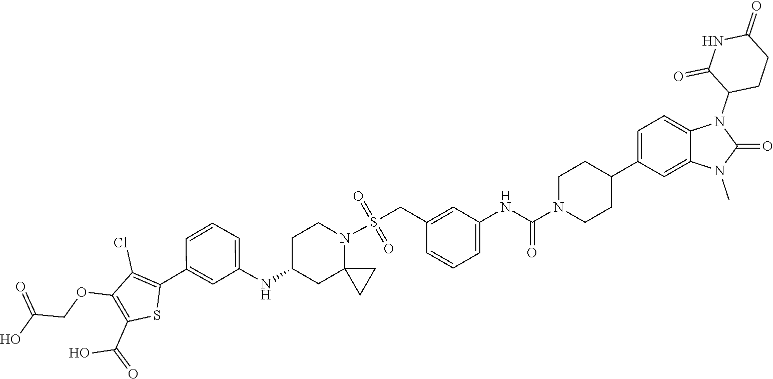 [SMILES: Cn1c(=O)n(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6)CC56CC6)c4)CC3)cc21] |
| 231 | 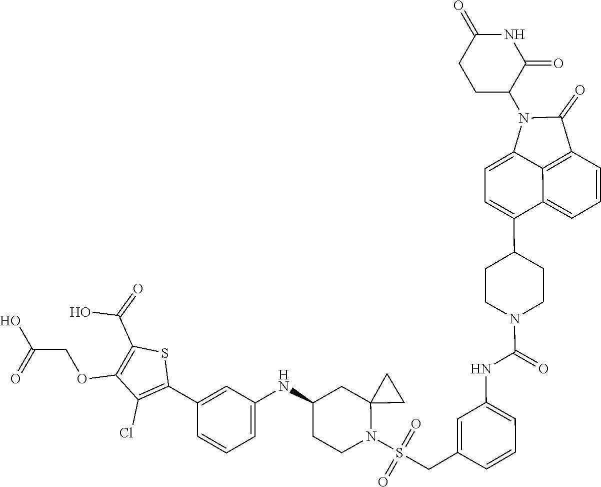 [SMILES: O=C(O)COc1c(C(=O)O)sc(-c2cccc(N[C@@H]3CCN(S(=O)(=O)Cc4cccc(NC(=O)N5CCC(c6ccc7c8c(cccc68)C(=O)N7C6CCC(=O)NC6=O)CC5)c4)C4(CC4)C3)c2)c1Cl] |
| 232 | 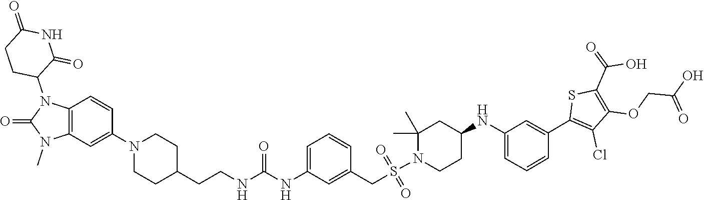 [SMILES: Cn1c(=O)n(C2CCC(=O)NC2=O)c2ccc(N3CCC(CCNC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 233 | 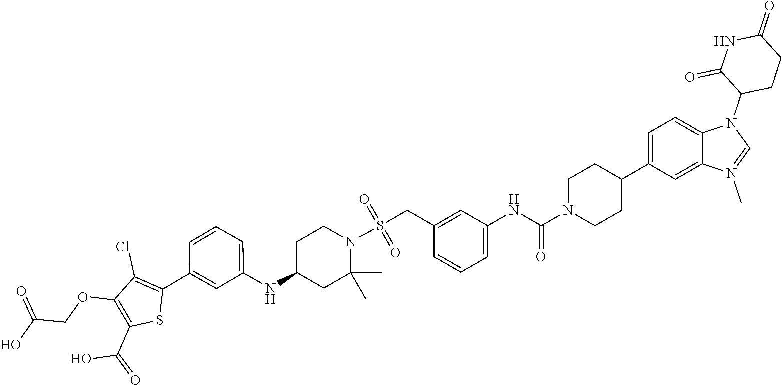 |
| 234 | 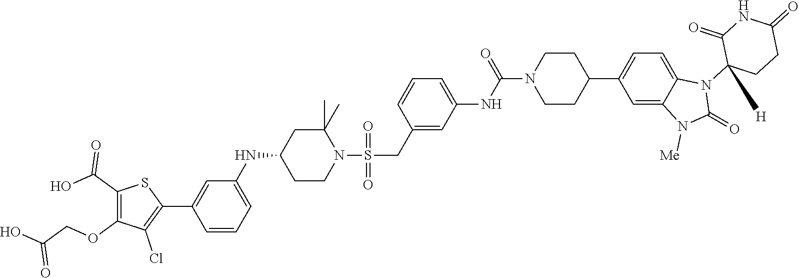 [SMILES: Cn1c(=O)n([C@H]2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 235 | 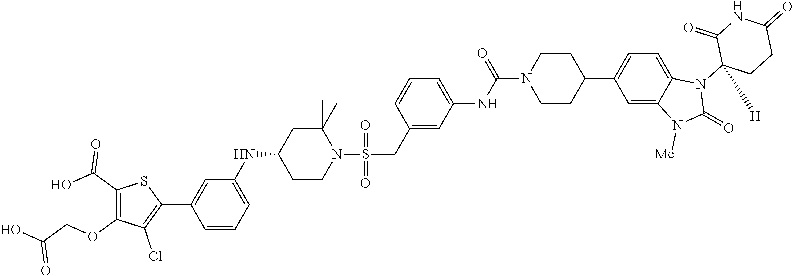 [SMILES: Cn1c(=O)n([C@@H]2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] |
| 236 | 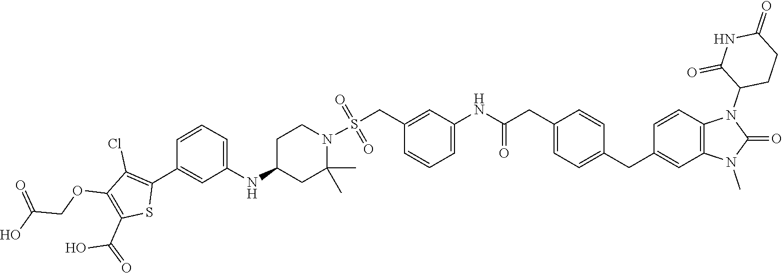 [SMILES: Cn1c(=O)n(C2CCC(=O)NC2=O)c2ccc(Cc3ccc(CC(=O)Nc4cccc(CS(=O)(=O)N5CC[C@H](Nc6cccc(-c7sc(C(=O)O)c(OCC(=O)O)c7Cl)c6)CC5(C)C)c4)cc3)cc21] |
| 237 | 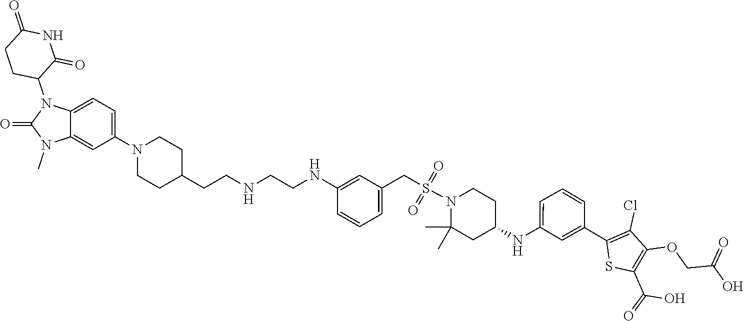 [SMILES: Cn1c(=O)n(C2CCC(=O)NC2=O)c2ccc(N3CCC(CCNCCNc4cccc(CS(=O)(=O)N5CC[C@H](Nc6cccc(-c7sc(C(=O)O)c(OCC(=O)O)c7Cl)c6)CC5(C)C)c4)CC3)cc21] |
| 238 | 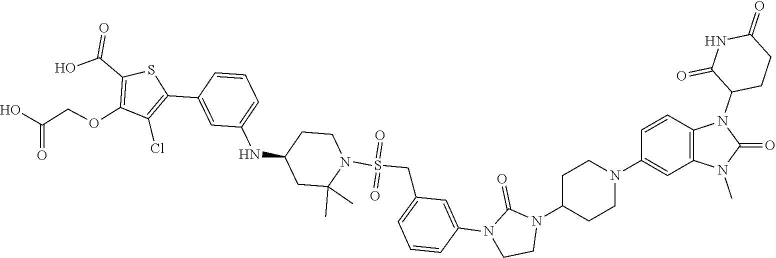 [SMILES: Cn1c(=O)n(C2CCC(=O)NC2=O)c2ccc(N3CCC(N4CCN(c5cccc(CS(=O)(=O)N6CC[C@H](Nc7cccc(-c8sc(C(=O)O)c(OCC(=O)O)c8Cl)c7)CC6(C)C)c5)C4=O)CC3)cc21] |

### Pharmaceutical Compositions

Some embodiments provide a pharmaceutical composition comprising a compound of Formula (I), or a pharmaceutically acceptable salt thereof.

### Methods of Treatment

The present disclosure features compounds, compositions, and methods comprising a compound of Formula (I). In some embodiments, the compounds, compositions, and methods described herein are used in the prevention or treatment of a disease. Exemplary diseases include, but are not limited to cancer, type-2 diabetes, metabolic syndrome, obesity, NAFLD, NASH, or another metabolic disease.

Some embodiments provide a method for treating cancer in a subject in need thereof, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof.

Some embodiments provide a method for inhibiting mammalian cell proliferation, comprising contacting the mammalian cell with an effective amount of a compound of Formula (I), or a pharmaceutically acceptable salt thereof.

Some embodiments provide a method for decreasing levels of a protein in a mammalian cell, comprising contacting the mammalian cell with an effective amount of a compound of Formula (I), or a pharmaceutically acceptable salt thereof; wherein the protein is PTPN1, PTPN2, or a combination thereof.

In some embodiments, the contacting occurs in vivo. In some embodiments, the contacting occurs in vitro.

In some embodiments, the mammalian cell is a mammalian cancer cell.

Some embodiments provide a method for inhibiting metastasis in a subject having a particular cancer in need of such treatment, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof.

Some embodiments provide a method for treating a metabolic disease in a subject in need thereof, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof.

In some embodiments, the metabolic disease is NAFLD, NASH, type 2 diabetes, or a combination of any of the foregoing.

In some embodiments, the metabolic disease is type 2 diabetes.

Some embodiments provide a method for decreasing BMI in a subject in need thereof, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof.

Some embodiments provide a method for inhibiting weight gain in a subject in need thereof, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof.

In some embodiments, the subject has an average BMI of between about 25 and about 45 prior to initiation of treatment with a compound of Formula (I), or a pharmaceutically acceptable salt thereof.

Some embodiments provide a method for increasing proliferation of mammalian T-cells in the presence of T-cell receptor stimulation, comprising contacting a mammalian thymus cell with an effective amount of a compound of Formula (I), or a pharmaceutically acceptable salt thereof; wherein the protein is PTPN1, PTPN2, or a combination thereof.

Some embodiments provide a method for activating mammalian T-cells in the presence of T-cell receptor stimulation, comprising contacting the mammalian T-cell with an effective amount of a compound of Formula (I), or a pharmaceutically acceptable salt thereof; wherein the protein is PTPN1, PTPN2, or a combination thereof.

In some embodiments, the contacting occurs in vivo. In some embodiments, the contacting occurs in vitro.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General scheme relating to methods of making exemplary compounds of the invention are additionally described in the section entitled Methods of Making Exemplary Compounds.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Preparation of Exemplary Intermediates

### 2-((5-(3-((1-((3-aminobenzyl)sulfonyl)piperidin-4-yl)amino)phenyl)-2-(tert-butoxycarbonyl)-4-chlorothiophen-3-yl)oxy)acetic acid (A-10)

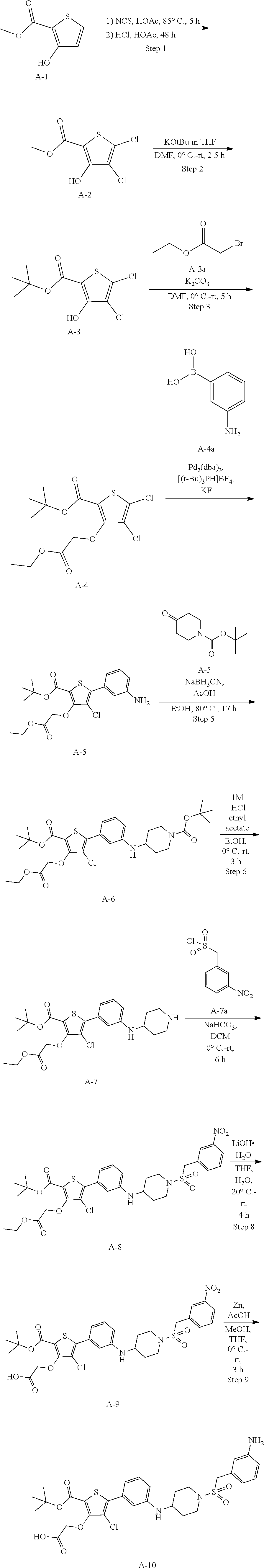

Step 1: methyl 4,5-dichloro-3-hydroxythiophene-2-carboxylate (A-2) To a well-stirred solution of methyl 3-hydroxythiophene-2-carboxylate (A-1, 13 g, 82.19 mmol) in acetic acid (65 mL) in a 250 mL three neck flask was added N-chlorosuccinimide (28.53 g, 213.69 mmol, 17.29 mL) slowly in portions. The resulting reaction mixture was heated at 85° C. for 5 h. Upon completion, the reaction mixture was cooled to ambient temperature and poured into ice water (225 mL) while stirring before extracting with diethyl ether (3×250 mL). The combined organic layers were washed with brine (2×250 mL) and then with aqueous sodium bicarbonate solution until the washings were basic. The organic layer was dried over anhydrous sodium sulfate, filtered and the solvent removed under reduced pressure. The residue was dissolved in acetic acid (45 mL) and dry HCl gas was bubbled through the mixture for 15 min. The reaction mixture was allowed to stand for 48 h at room temperature. The solvent was decanted and water (250 mL) was added, stirred and filtered to afford methyl 4,5-dichloro-3-hydroxythiophene-2-carboxylate (A-2, 9.5 g, 41.0 mmol, 50% yield) as a light yellow solid. LCMS (ES−): m/z 224.9 [M−H]⁻.

### Step 2: tert-butyl 4,5-dichloro-3-hydroxythiophene-2-carboxylate (A-3)

To a well-stirred solution of potassium tert-butoxide (49.42 g, 440.40 mmol) in DMF (60 mL) was added a solution of methyl 4,5-dichloro-3-hydroxy-thiophene-2-carboxylate (A-2, 10 g, 44.04 mmol) in DMF (60 mL) dropwise over a period of 30 min at 0° C. The reaction mixture was allowed to come to room temperature and stirred for 2.5 h. The reaction mixture was quenched with ice water (100 mL) and extracted with ethyl acetate (3×100 mL). The combined organic layers were washed with water (300 mL) and brine (200 mL), dried over anhydrous sodium sulfate and filtered. The solvent was removed under reduced pressure and the residue was purified by flash silica gel column chromatography (1-2% Ethyl acetate in petroleum ether) to afford tert-butyl 4,5-dichloro-3-hydroxythiophene-2-carboxylate (A-3, 8.5 g, 30.0 mmol, 68% yield) as a beige solid. LCMS (ES−): m/z 267.0 [M−H]⁻.

### Step 3: tert-butyl 4,5-dichloro-3-(2-ethoxy-2-oxoethoxy)thiophene-2-carboxylate (A-4)

To a solution of tert-butyl 4,5-dichloro-3-hydroxythiophene-2-carboxylate (A-3, 17 g, 63.16 mmol) in DMF (150 mL) in a 250 mL round bottom flask was added anhydrous potassium carbonate (17.46 g, 126.33 mmol) and the suspension was cooled to 0° C. Then ethyl bromoacetate (A-3a, 15.82 g, 94.74 mmol, 10.48 mL) was added dropwise over a period of 5 min. The reaction mixture was stirred for 5 h at room temperature. The reaction mixture was quenched with water (600 mL) and extracted with diethyl ether (3×150 mL). The combined organic layers were washed with brine (250 mL), dried over anhydrous sodium sulfate, filtered and the solvent removed under reduced pressure. The residue was purified by flash silica gel column chromatography (5-8% Ethyl acetate in petroleum ether) to afford tert-butyl 4,5-dichloro-3-(2-ethoxy-2-oxoethoxy)thiophene-2-carboxylate (A-4, 20 g, 56.30 mmol, 59% yield) as an off white solid. LCMS (ES+): m/z 299.0 [M−tBu+H]⁺.

### Step 4: tert-butyl 5-(3-aminophenyl)-4-chloro-3-(2-ethoxy-2-oxo-ethoxy)thiophene-2-carboxylate (A-5)

To a solution of tert-butyl 4,5-dichloro-3-(2-ethoxy-2-oxo-ethoxy)thiophene-2-carboxylate (A-4, 10 g, 28.15 mmol) and (3-aminophenyl)boronic acid (A-4a, 7.71 g, 56.30 mmol) in 1,4-dioxane (75 mL) in a pressure tube was added potassium fluoride (4.91 g, 84.45 mmol) and the mixture was purged by bubbling nitrogen gas through for 5 min. Then tri-tert-butylphosphonium tetrafluoroborate (816.72 mg, 2.82 mmol) and tris(dibenzylideneacetone) dipalladium (0) (1.29 g, 1.41 mmol) were added and the tube was sealed. The reaction mixture was heated at 70° C. for 17 h. The reaction mixture was cooled to room temperature, diluted with 1,4-dioxane and filtered through Celite. The filtrate was concentrated under reduced pressure, and the residue purified by flash silica gel column chromatography (10% Ethyl acetate in petroleum ether) to afford tert-butyl 5-(3-aminophenyl)-4-chloro-3-(2-ethoxy-2-oxo-ethoxy)thiophene-2-carboxy late (A-5, 5.5 g, 12.47 mmol, 44% yield) as a pale yellow solid. LCMS (ES+): m/z 356.0 [M−tBu+H]⁺.

### Step 5: tert-butyl 4-((3-(5-(tert-butoxycarbonyl)-3-chloro-4-(2-ethoxy-2-oxoethoxy)thiophen-2-yl)phenyl)amino)piperidine-1-carboxylate (A-6)

To a solution of tert-butyl 5-(3-aminophenyl)-4-chloro-3-(2-ethoxy-2-oxo-ethoxy)thiophene-2-carboxylate (A-5, 5 g, 12.14 mmol) in ethanol (50 mL) in a pressure tube was added tert-butyl 4-oxopiperidine-1-carboxylate (A-5a, 12.09 g, 60.69 mmol) followed by acetic acid (3.64 g, 60.69 mmol, 3.47 mL). The tube was sealed and the reaction mixture heated at 80° C. for 3 h. The reaction mixture was cooled to room temperature and sodium cyanoborohydride (3.81 g, 60.69 mmol) added in portions. The reaction mixture was heated at 80° C. for 17 h. The solvent was removed under reduced pressure and the residue diluted with ethyl acetate (100 mL). The organic layer was washed with water (75 mL) and brine solution (75 mL), dried over anhydrous sodium sulfate and filtered. The solvent was removed under reduced pressure and the residue purified by flash silica gel column chromatography (8-10% Ethyl acetate in petroleum ether) to afford tert-butyl 4-((3-(5-(tert-butoxycarbonyl)-3-chloro-4-(2-ethoxy-2-oxoethoxy)thiophen-2-yl)phenyl)amino)piperidine-1-carboxylate (A-6, 5.5 g, 9.00 mmol, 74% yield) as a yellow solid. LCMS (ES+): m/z 439.0 [M−tBu+H]⁺.

### Step 6: tert-butyl 4-chloro-3-(2-ethoxy-2-oxoethoxy)-5-(3-(piperidin-4-ylamino)phenyl)thiophene-2-carboxylate hydrochloride (A-7)

Into a 250 mL multi-neck round bottom flask containing a well-stirred solution of tert-butyl 4-((3-(5-(tert-butoxycarbonyl)-3-chloro-4-(2-ethoxy-2-oxoethoxy)thiophen-2-yl)phenyl)amino)piperidine-1-carboxylate (A-6, 5.63 g, 9.46 mmol) in ethanol (25 mL) was added 1M HCl in ethyl acetate (50 mL) at 0° C. and the mixture was allowed to come to room temperature and stirred for 2 h. The volatiles were removed under reduced pressure to afford tert-butyl 4-chloro-3-(2-ethoxy-2-oxoethoxy)-5-(3-(piperidin-4-ylamino)phenyl)thiophene-2-carboxylate hydrochloride (A-7, 4.9 g, 9.89 mmol, 90% yield) as a pale yellow solid. LCMS (ES+): m/z 495.0 [M+H]⁺.

### Step 7: tert-butyl 4-chloro-3-(2-ethoxy-2-oxo-ethoxy)-5-[3-[[1-[(3-nitrophenyl)methylsulfonyl]-4-piperidyl]amino]phenyl]thiophene-2-carboxylate (A-8)

To a solution of tert-butyl 4-chloro-3-(2-ethoxy-2-oxoethoxy)-5-(3-(piperidin-4-ylamino)phenyl)thiophene-2-carboxylate hydrochloride (A-7, 4.9 g, 9.22 mmol) in a mixture of DCM (150 mL) and saturated aqueous sodium bicarbonate solution (150 mL) was added (3-nitrophenyl)methanesulfonyl chloride (A-7a, 2.61 g, 11.06 mmol) slowly in portions at 0° C. The reaction was stirred at room temperature for 6 h. The organic layer was separated, and the aqueous layer extracted with DCM (2×100 mL). The combined organic layers were washed with water (150 mL), saturated brine (150 mL), dried over anhydrous sodium sulfate and filtered. The solvent was removed under reduced pressure and the residue purified by flash silica gel column chromatography (20-25% Ethyl acetate in petroleum ether) to afford tert-butyl 4-chloro-3-(2-ethoxy-2-oxo-ethoxy)-5-[3-[[1-[(3-nitrophenyl)methylsulfonyl]-4-piperidyl]amino]phenyl]thiophene-2-carboxylate (A-8, 5.4 g, 7.55 mmol, 82% yield) as a yellow solid. LCMS (ES+): m/z 638.1 [M−tBu+H]⁺.

### Step 8: 2-((2-(tert-butoxycarbonyl)-4-chloro-5-(3-((1-((3-nitrobenzyl)sulfonyl)piperidin-4-ylamino)phenyl)thiophen-3-yl)oxy)acetic acid (A-9)

To a solution of tert-butyl 4-chloro-3-(2-ethoxy-2-oxo-ethoxy)-5-[3-[[1-[(3-nitrophenyl)methylsulfonyl]-4-piperidyl]amino]phenyl]thiophene-2-carboxylate (A-8, 5.3 g, 7.63 mmol) in a mixture of THF (120 mL) and water (40 mL) at 0° C. was added LiOH monohydrate (1.60 g, 38.17 mmol) portion wise and stirred at room temperature for 4 h. The reaction mixture was concentrated under reduced pressure and diluted with water (100 mL). The mixture was cooled to 0° C. and carefully acidified with 1.5 N HCl. The reaction was stirred for 30 min and the precipitate was filtered to afford 2-((2-(tert-butoxycarbonyl)-4-chloro-5-(3-((1-((3-nitrobenzyl)sulfonyl)piperidin-4-yl)amino)phenyl)thiophen-3-yl)oxy)acetic acid (A-9, 4.5 g, 6.6 mmol, 92% yield) as an off white solid. The material was used in the next step without further purification. LCMS

(ES+): m/z 666.3 [M+H]⁺.

### Step 9:2-((5-(3-((1-((3-aminobenzyl)sulfonyl)piperidin-4-yl)amino)phenyl)-2-(tert-butoxycarbonyl)-4-chlorothiophen-3-yl)oxy)acetic acid (A-10)

To a solution of 2-((2-(tert-butoxycarbonyl)-4-chloro-5-(3-((1-((3-nitrobenzyl)sulfonyl)piperidin-4-yl)amino)phenyl)thiophen-3-yl)oxy)acetic acid (A-9, 4.6 g, 6.91 mmol) in a mixture of methanol (50 mL) and THF (50 mL) at 0° C. was added zinc powder (325 mesh) (2.26 g, 34.53 mmol) and acetic acid (4.15 g, 69.05 mmol, 3.95 mL) dropwise over a period of 5 min. After 3 h, the reaction mixture was diluted with THF (50 mL) and filtered through Celite. The filtrate was concentrated under reduced pressure, diluted with ethyl acetate (100 mL) and washed with sodium bicarbonate solution (40 mL), followed by water (40 mL) and brine solution (40 mL). The mixture was dried over anhydrous sodium sulfate, filtered and solvent removed under reduced pressure. The residue was purified by reverse phase prep HPLC to obtain 2-((5-(3-((1-((3-aminobenzyl)sulfonyl)piperidin-4-yl)amino)phenyl)-2-(tert-butoxycarbonyl)-4-chlorothiophen-3-yl)oxy)acetic acid (A-10, 3.8 g, 5.92 mmol, 86% yield) as an off white solid. LCMS (ES−): m/z 634.1 [M−H]⁻. ¹H NMR (400 MHz, DMSO-d6): δ 7.21 (t, J=10.40 Hz, 1H), 7.00 (t, J=10.40 Hz, 1H), 6.88 (s, 1H), 6.81 (d, J=10.00 Hz, 1H), 6.70 (d, J=11.20 Hz, 1H), 6.59-6.61 (m, 1H), 6.53 (dd, J=2.40, 10.20 Hz, 2H), 5.90 (d, J=10.40 Hz, 1H), 4.83 (s, 2H), 4.19 (s, 2H), 3.39-3.56 (m, 3H), 2.89-2.96 (m, 2H), 1.92-1.97 (m, 2H), 1.52 (s, 9H), 1.34-1.38 (m, 2H).

### 2,2-dimethyl-1-((3-nitrobenzyl)sulfonyl)piperidin-4-one (A-13)

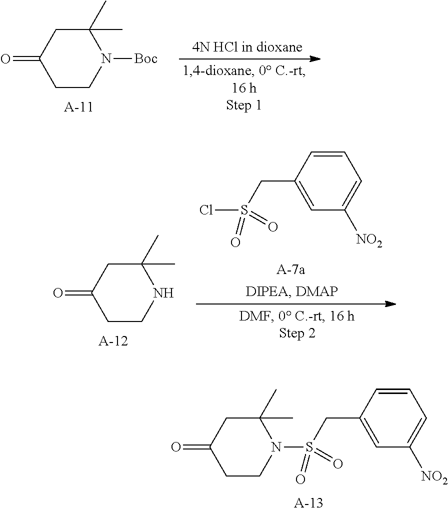

### Step 1: 2,2-dimethylpiperidin-4-one hydrochloride (A-12)

To a 250 mL single neck round bottom flask containing a well-stirred suspension of tert-butyl 2,2-dimethyl-4-oxo-piperidine-1-carboxylate (A-11, 15 g, 65.99 mmol) in 1,4-dioxane (150 mL) was added 4 M HCl in dioxane (129.77 g, 600.00 mmol, 250 mL) dropwise at 0° C. The mixture was stirred at room temperature for 16 h. The reaction mixture was concentrated under reduced pressure to afford 2,2-dimethylpiperidin-4-one hydrochloride (A-12, 11 g, crude) as an off-white solid. The material was used in the next step without further purification.

### Step 2: 2,2-dimethyl-1-[(3-nitrophenyl)methylsulfonyl]piperidin-4-one (A-13)

To a well-stirred suspension of 2,2-dimethylpiperidin-4-one hydrochloride (A-12, 11 g, 67.22 mmol) in DMF (200 mL) in a 500 mL round bottom flask were added DIPEA (13.03 g, 100.83 mmol, 17.56 mL) and DMAP (821.21 mg, 6.72 mmol) at room temperature followed by (3-nitrophenyl)methanesulfonyl chloride (A-7a, 23.76 g, 100.83 mmol) at 0° C. The resulting mixture was stirred at room temperature for 16 h. The reaction mixture was concentrated under reduced pressure, diluted with water (500 mL) and extracted with dichloromethane (550 mL). The organic layer was washed with brine solution (400 mL), dried over sodium sulfate, filtered and concentrated under reduced pressure. The residue was purified by flash silica gel column chromatography (40% Ethyl acetate in petroleum ether) to afford 2,2-dimethyl-1-[(3-nitrophenyl)methylsulfonyl]piperidin-4-one (A-13, 7.8 g, 12.67 mmol, 19% yield) as a pale yellow solid. LCMS (ES+): m/z 327.1 [M+H]⁺.

### 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a)

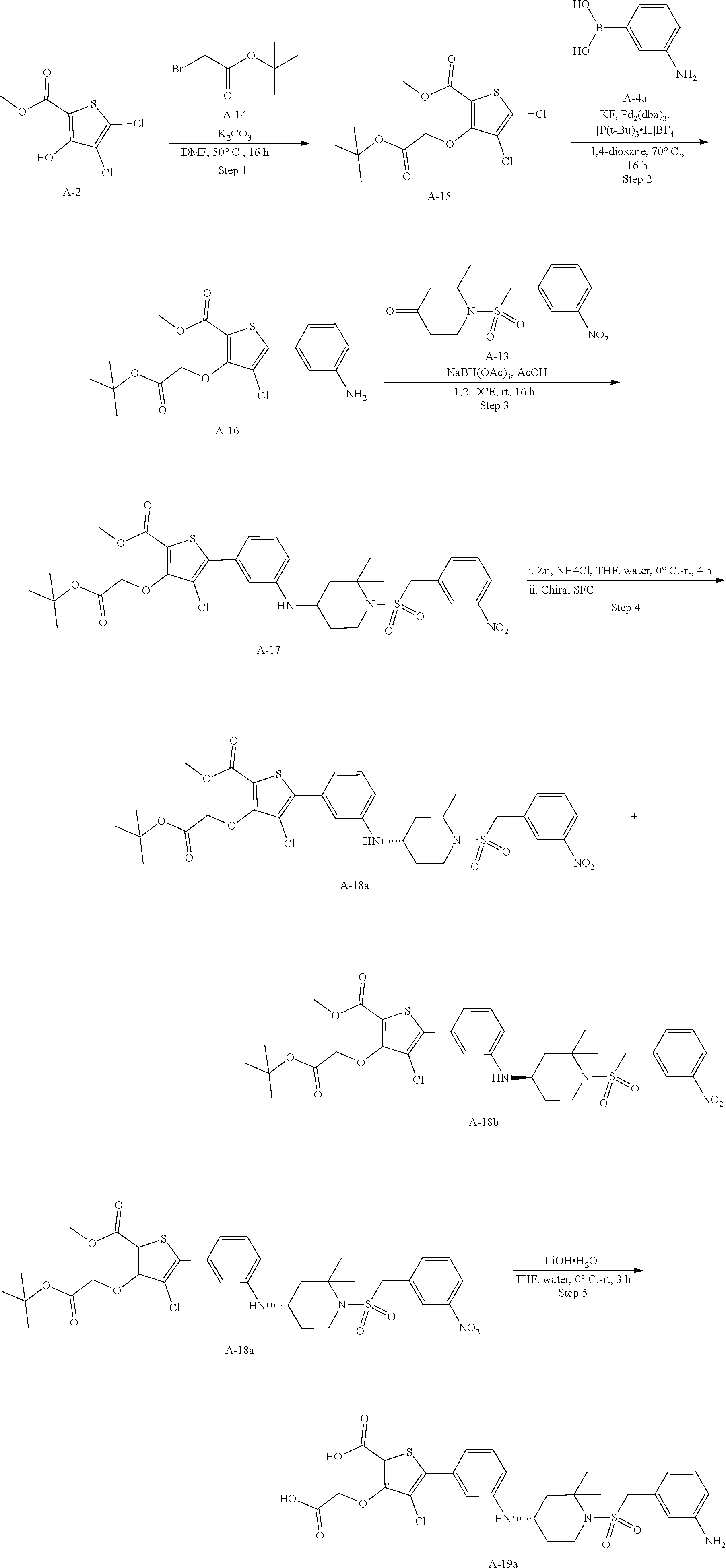

### Step 1: methyl 3-(2-tert-butoxy-2-oxo-ethoxy)-4,5-dichloro-thiophene-2-carboxylate A-15)

Methyl 3-(2-tert-butoxy-2-oxo-ethoxy)-4,5-dichloro-thiophene-2-carboxylate (A-15, 12.4 g, 34.52 mmol, 71% yield) was synthesized from methyl 4,5-dichloro-3-hydroxy-thiophene-2-carboxylate (A-2) and tert-butyl 2-bromoacetate (A-14) in a similar fashion to Compound A-4, except using 1.2 eq. A-14. LCMS (ES+): m/z 284.9 [M−tBu+H]⁺.

### Step 2: methyl 5-(3-aminophenyl)-3-(2-tert-butoxy-2-oxo-ethoxy)-4-chloro-thiophene-2-carboxylate (A-16)

Methyl 5-(3-aminophenyl)-3-(2-tert-butoxy-2-oxo-ethoxy)-4-chloro-thiophene-2-carboxylate (A-16, 6.2 g, crude) was synthesized from methyl 3-(2-tert-butoxy-2-oxo-ethoxy)-4,5-dichloro-thiophene-2-carboxy late (A-15) and (3-aminophenyl)boronic acid (A-4a) in a similar fashion to Compound A-5, except using 1 eq. A-4a and 3 eq. potassium fluoride. The material was used in the next step without purification. LCMS (ES+): m/z 342.1 [M+H]⁺.

### Step 3: methyl 3-(2-(tert-butoxy)-2-oxoethoxy)-4-chloro-5-(3-((2,2-dimethyl-1-((3-nitrobenzyl)sulfonyl)piperidin-4-yl)amino)phenyl)thiophene-2-carboxylate (A-17)

Into a 500 mL two neck round bottom flask containing a well-stirred suspension of methyl 5-(3-aminophenyl)-3-(2-tert-butoxy-2-oxo-ethoxy)-4-chloro-thiophene-2-carboxylate (A-16, 9.5 g, 23.88 mmol) in DCE (250 mL) was added acetic acid (3.58 g, 59.69 mmol, 3.41 mL), 2,2-dimethyl-1-((3-nitrobenzyl)sulfonyl)piperidin-4-one (A-13, 7.79 g, 23.88 mmol) and sodium triacetoxy borohydride (50.60 g, 238.77 mmol). After 16 h the reaction mixture was diluted with water (350 mL), extracted with dichloromethane (450 mL) and washed with brine solution (300 mL). The organic layer was dried over sodium sulfate, filtered and concentrated under reduced pressure. The residue was purified by flash silica gel column chromatography (30% Ethyl acetate in petroleum ether) to afford methyl 3-(2-tert-butoxy-2-oxo-ethoxy)-4-chloro-5-[3-[[2,2-dimethyl-1-[(3-nitrophenyl)methylsulfonyl]-4-piperidyl]amino]phenyl]thiophene-2-carboxylate (A-17, 7.1 g, 8.72 mmol, 37% yield) as a pale yellow solid. LCMS (ES+): m/z 708.1 [M+H]⁺.

### Step 4: methyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (A-18a, First Eluted Fraction) and methyl 5-[3-[[(4R)-1-[(3-aminophenyl)methylsulfonyl]-2,2-dimethyl-4-piperidyl]amino]phenyl]-3-(2-tert-butoxy-2-oxo-ethoxy)-4-chlorothiophene-2-carboxylate (A-18b, Second Eluted Fraction)

Into a 250 mL two neck round bottom flask containing a well-stirred suspension of methyl 3-(2-tert-butoxy-2-oxo-ethoxy)-4-chloro-5-[3-[[2,2-dimethyl-1-[(3-nitrophenyl)methylsulfonyl]-4-piperidyl]amino]phenyl]thiophene-2-carboxylate (A-17, 5.31 g, 7.49 mmol) in THF (60 mL) and water (60 mL) was added zinc powder (4.90 g, 74.90 mmol) in portions at room temperature. The mixture was cooled to 0° C. and ammonium chloride (4.01 g, 74.90 mmol) was added in portions. The reaction mixture was stirred at room temperature for 4 h. The reaction mixture was diluted with THF (200 mL) and filtered through Celite, washing with acetone (100 mL). The filtrate was concentrated under reduced pressure, the residue taken up in dichloromethane (400 mL) and washed with water (350 mL) and brine solution (300 mL). The organic layer was dried over anhydrous sodium sulfate, filtered and the solvent removed under reduced pressure. The residue was purified by flash silica gel column chromatography (40% Ethyl acetate in petroleum ether) to afford methyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (18a/b, 4.2 g, 5.57 mmol, 74% yield) as an off-white solid. The enantiomers were separated by chiral SFC: Method details: Column Name: YMC Amylose-SA; Co-Solvent: 40% and Co-Solvent Name: IPA; Outlet Pressure: 100 bar; Temperature: 35° C. After concentration, the first eluted fraction at RT 3.7 min: methyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (A-18a, first eluted fraction, 2 g, 2.65 mmol, 35% yield) was isolated as an off-white solid. LCMS (ES+): m/z 678.1 [M+H]⁺.

Second eluted fraction at RT 5.37 min: methyl 5-[3-[[(4R)-1-[(3-aminophenyl)methylsulfonyl]-2,2-dimethyl-4-piperidyl]amino]phenyl]-3-(2-tert-butoxy-2-oxo-ethoxy)-4-chlorothiophene-2-carboxylate (A-18b, second eluted fraction, 2.2 g, 2.92 mmol, 39% yield) was isolated as an off-white solid. LCMS (ES+): m/z 678.1 [M+H]⁺.

### Step 5: 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a)

5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 1.75 g, 2.76 mmol, 94% yield) was synthesized from methyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (A-18a) in a similar fashion to Compound A-9, except using 3 eq. LiOH monohydrate. The product was purified by reverse phase prep HPLC [Prep Method: column: XSELECT-C18 150 MM, Mobile phase: 0.1% TFA in Water/MeCN]. LCMS (ES+): m/z 608.3 [M+H]⁺. ¹H NMR (400 MHz, DMSO-d6): δ 7.30-7.32 (m, 1H), 7.19-7.21 (m, 1H), 7.04-7.10 (m, 3H), 6.89 (s, 1H), 6.82 (d, J=10.40 Hz, 1H), 6.71 (d, J=10.00 Hz, 1H), 4.92 (s, 2H), 4.29-4.34 (m, 2H), 3.44-3.49 (m, 2H), 0.17 (s, 2H), 3.10-3.14 (m, 1H), 1.78-1.82 (m, 2H), 1.48 (s, 3H), 1.40 (s, 3H), 1.09-1.12 (m, 1H).

### 5-[3-[[(4R)-1-[(3-aminophenyl)methylsulfonyl]-2,2-dimethyl-4-piperidyl]amino]phenyl]-3-(carboxymethoxy)-4-chloro-thiophene-2-carboxylic acid (A-19b)

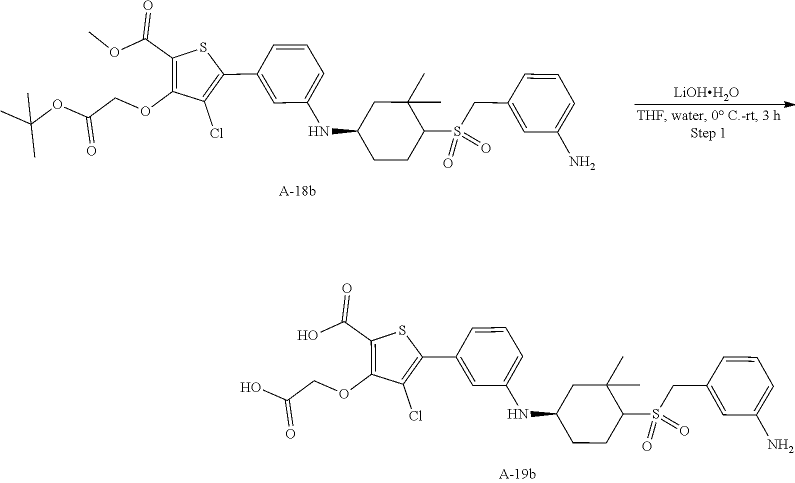 [SMILES: COC(=O)c1sc(-c2cccc(N[C@@H]3CCC(S(=O)(=O)Cc4cccc(N)c4)C(C)(C)C3)c2)c(Cl)c1OCC(=O)OC(C)(C)C ; "LiOH·H2O" ; CC1(C)C[C@H](Nc2cccc(-c3sc(C(=O)O)c(OCC(=O)O)c3Cl)c2)CCC1S(=O)(=O)Cc1cccc(N)c1]

### Step 1: 5-[3-[[(4R)-1-[(3-aminophenyl)methylsulfonyl]-2,2-dimethyl-4-piperidyl]amino]phenyl]-3-(carboxymethoxy)-4-chloro-thiophene-2-carboxylic acid (A-19b)

5-[3-[[(4R)-1-[(3-aminophenyl)methylsulfonyl]-2,2-dimethyl-4-piperidyl]amino]phenyl]-3-(carboxymethoxy)-4-chloro-thiophene-2-carboxylic acid (A-19b, 110 mg, 135.7 μmol, 58% yield) was synthesized from methyl 5-[3-[[(4R)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (A-18b) in a similar fashion to Compound A-9, except using 2 eq. LiOH monohydrate. The product was purified by reverse phase prep HPLC [Prep Method: column: X-SELECT-C18 150 MM, Mobile phase: 0.1% TFA in Water/MECN]. LCMS (ES+): m/z 608.1 [M+H]⁺.

### 5-[3-[[1-[1-(3-aminophenyl)-1-methyl-ethyl]sulfonyl-4-piperidyl]amino]phenyl]-3-(carboxymethoxy)-4-chloro-thiophene-2-carboxylic acid (A-26)

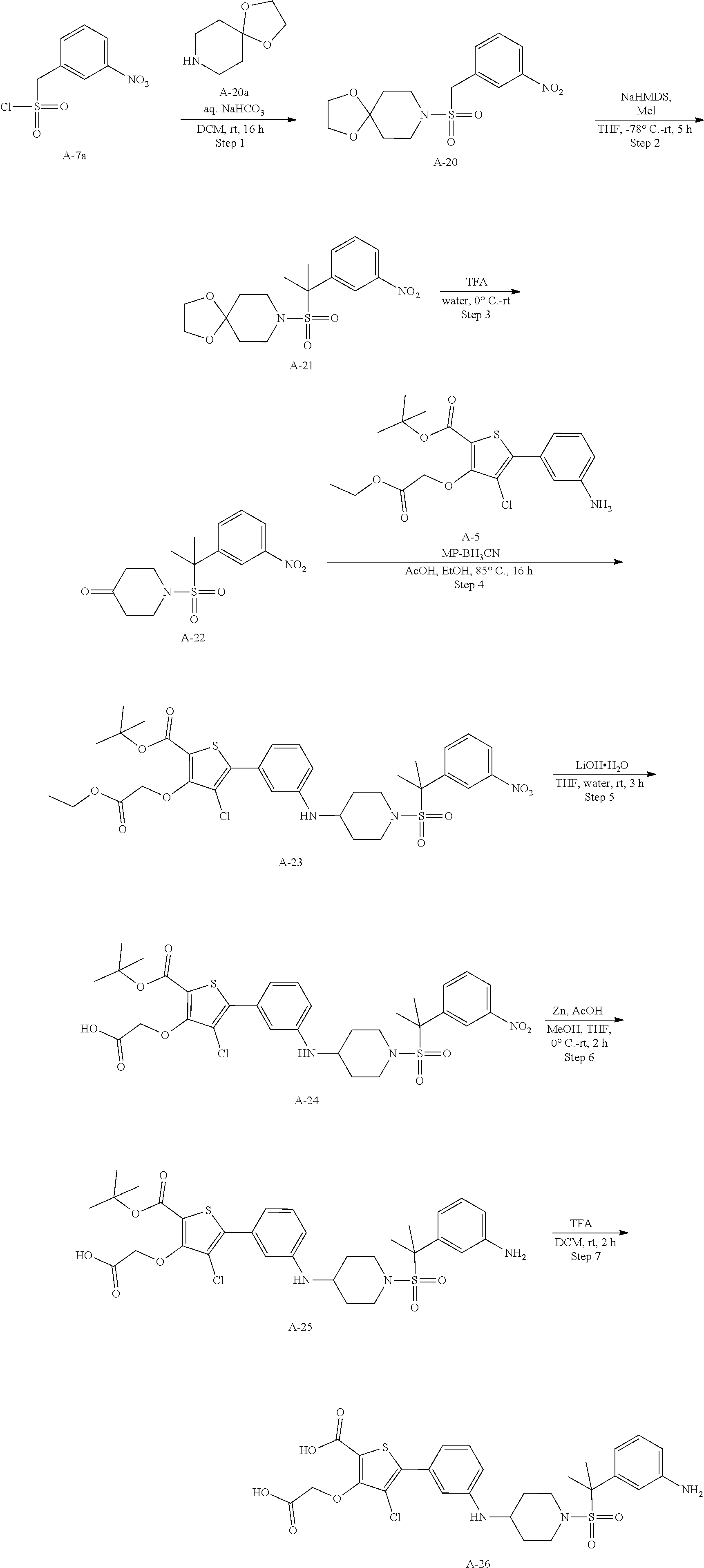

### Step 1:8-[(3-nitrophenyl)methylsulfonyl]-1,4-dioxa-8-azaspiro[4.5]decane (A-20)

Into a 100 mL three neck round bottom flask containing a well-stirred solution of 1,4-dioxa-8-azaspiro[4.5]decane (A-20a, 2 g, 13.97 mmol, 1.79 mL) in saturated aqueous sodium bicarbonate (15 mL) and DCM (15 mL) was added (3-nitrophenyl)methane sulfonyl chloride (A-7a, 3.29 g, 13.97 mmol) at 0° C. The reaction mixture was stirred at room temperature for 16 h. Water (100 mL) was added to the mixture and the aqueous phase was extracted with DCM (2×100 mL). The organic layer was washed with brine (50 mL), dried over anhydrous Sodium sulfate, filtered and the solvent removed under reduced pressure to afford 8-[(3-nitrophenyl)methylsulfonyl]-1,4-dioxa-8-azaspiro[4.5]decane (A-20, 3.2 g, 8.97 mmol, 64% yield) as a pale yellow solid. The material was used in the next step without further purification. LCMS (ES+): m/z 343.0 [M+H]⁺.

### Step 2:8-[1-methyl-1-(3-nitrophenyl)ethyl]sulfonyl-1,4-dioxa-8-azaspiro[4.5]decane (A-21)

Into a 100 mL three neck round bottom flask containing a well-stirred solution of 8-[(3-nitrophenyl)methylsulfonyl]-1,4-dioxa-8-azaspiro[4.5]decane (A-20, 2 g, 5.84 mmol) in THF (25 mL) at −78° C. were added sodium bis(trimethylsilyl)amide solution (14.60 mmol, 8 mL) and methyl iodide (2.07 g, 14.60 mmol, 909.17 μL). The resulting mixture was stirred at room temperature for 16 h. The reaction was quenched with aqueous ammonium chloride solution (50 mL) and extracted with ethyl acetate (2×100 mL). The organic layer was dried over Sodium sulfate, filtered and concentrated under reduced pressure. The residue was purified by column chromatography (30-35% ethyl acetate in pet-ether) to afford 8-[1-methyl-1-(3-nitrophenyl)ethyl]sulfonyl-1,4-dioxa-8-azaspiro[4.5]decane (A-21, 700 mg, 1.21 mmol, 21% yield) as a pale yellow solid. LCMS (ES+): m/z 393.4 [M+Na]⁺.

### Step 3: 1-[1-methyl-1-(3-nitrophenyl)ethyl]sulfonylpiperidin-4-one (A-22)

To a suspension of 8-[1-methyl-1-(3-nitrophenyl)ethyl]sulfonyl-1,4-dioxa-8-azaspiro[4.5]decane (A-21, 700 mg, 1.89 mmol) in water (3 mL) was added trifluoroacetic acid (20.72 g, 181.72 mmol, 14.00 mL) at 0° C. The reaction mixture was stirred at room temperature for 1 h. The reaction mixture was cooled to 0° C. and neutralized with aqueous sodium bicarbonate solution. The product was extracted with ethyl acetate (2×50 mL). The combined organic layers were dried over anhydrous Sodium sulfate, filtered and concentrated under reduced pressure to afford 1-[1-methyl-1-(3-nitrophenyl)ethyl]sulfonylpiperidin-4-one (A-22, 700 mg, 1.24 mmol, 66% yield). The material was used in the next step without further purification. LCMS (ES+): m/z 349.3 [M+Na]⁺.

### Step 4: tert-butyl 4-chloro-3-(2-ethoxy-2-oxoethoxy)-5-(3-((1-((2-(3-nitrophenyl)propan-2-yl)sulfonyl)piperidin-4-yl)amino)phenyl)thiophene-2-carboxylate (A-23)

Into a 50 mL round bottom flask containing a well-stirred solution of tert-butyl 5-(3-aminophenyl)-4-chloro-3-(2-ethoxy-2-oxo-ethoxy)thiophene-2-carboxylate (A-5, 550 mg, 1.34 mmol) in ethanol (15 mL) was added 1-[1-methyl-1-(3-nitrophenyl)ethyl]sulfonylpiperidin-4-one (A-22, 737.49 mg, 1.47 mmol), MP-cyanoborohydride (1.22 g, 10.68 mmol) and acetic acid (8.02 mg, 133.53 μmol, 7.64 μL). The reaction mixture was heated at 85° C. for 16 h. The reaction mixture was filtered through a cotton plug and the filtrate was evaporated to afford tert-butyl 4-chloro-3-(2-ethoxy-2-oxo-ethoxy)-5-[3-[[1-[1-methyl-1-(3-nitrophenyl)ethyl]sulfonyl-4-piperidyl]amino]phenyl]thiophene-2-carboxylate (A-23, 550 mg, 494.97 μmol, 37% yield) as an off-white solid. The material was used in the next step without further purification. LCMS (ES+): m/z 724.50 [M+H]⁺.

### Step 5: 2-[[2-tert-butoxycarbonyl-4-chloro-5-[3-[[1-[1-methyl-1-(3-nitrophenyl)ethyl]sulfonyl-4-piperidyl]amino]phenyl]-3-thienyl]oxy]acetic acid (A-24)

2-[[2-tert-butoxycarbonyl-4-chloro-5-[3-[[1-[1-methyl-1-(3-nitrophenyl)ethyl]sulfonyl-4-piperidyl]amino]phenyl]-3-thienyl]oxy]acetic acid (A-24, 500 mg, crude, purity 55%) was synthesized from tert-butyl 4-chloro-3-(2-ethoxy-2-oxo-ethoxy)-5-[3-[[1-[1-methyl-1-(3-nitrophenyl)ethyl]sulfonyl-4-piperidyl]amino]phenyl]thiophene-2-carboxylate (A-23) in a similar fashion to Compound A-9, except using 3 eq. LiOH monohydrate. The material was used in the next step without further purification. LCMS (ES+): m/z 694.80 [M+H]⁺.

### Step 6: 2-[[5-[3-[[1-[1-(3-aminophenyl)-1-methyl-ethyl]sulfonyl-4-piperidyl]amino]phenyl]-2-tert-butoxycarbonyl-4-chloro-3-thienyl]oxy]acetic acid (A-25)

2-[[5-[3-[[1-[1-(3-aminophenyl)-1-methyl-ethyl]sulfonyl-4-piperidyl]amino]phenyl]-2-tert-butoxycarbonyl-4-chloro-3-thienyl]oxy]acetic acid (A-25, 300 mg, 293.57 μmol, 75% yield) was synthesized from 2-[[2-tert-butoxycarbonyl-4-chloro-5-[3-[[1-[1-methyl-1-(3-nitrophenyl)ethyl]sulfonyl-4-piperidyl]amino]phenyl]-3-thienyl]oxy]acetic acid (A-24) in a similar fashion to Compound A-10, except using 3 eq. Zinc and 4 eq. Acetic acid. Upon completion, the reaction mixture was filtered through Celite, and the filtrate concentrated under reduced pressure. The residue was washed with aqueous sodium bicarbonate solution and extracted with 10% MeOH/DCM (2×25 mL). The combined organic layers were dried over anhydrous Sodium sulfate, filtered and concentrated under reduced pressure. The material was used in the next step without purification. LCMS (ES+): m/z 665.1 [M+H]⁺.

### Step 7: 5-[3-[[1-[1-(3-aminophenyl)-1-methyl-ethyl]sulfonyl-4-piperidyl]amino]phenyl]-3-(carboxymethoxy)-4-chloro-thiophene-2-carboxylic acid (A-26)

Into a 25 mL three neck round bottom flask containing a well-stirred solution of 2-[[5-[3-[[1-[1-(3-aminophenyl)-1-methyl-ethyl]sulfonyl-4-piperidyl]amino]phenyl]-2-tert-butoxycarbonyl-4-chloro-3-thienyl]oxy]acetic acid (A-25, 50 mg, 75.28 μmol) in DCM (2 mL) was added trifluoroacetic acid (25.75 mg, 225.83 μmol, 17.40 μL) at 0° C. The reaction mixture was stirred at room temperature for 2 h. The solvent was evaporated and the residue triturated with diethyl ether (2×5 mL) to afford 5-[3-[[1-[1-(3-aminophenyl)-1-methyl-ethyl]sulfonyl-4-piperidyl]amino]phenyl]-3-(carboxymethoxy)-4-chloro-thiophene-2-carboxylic acid (A-26, 50 mg, 92% yield) as a brown solid. LCMS (ES+): m/z 608.00 [M+H]⁺. ¹H NMR (400 MHz, DMSO-d6): δ 7.16-7.17 (m, 1H), 7.00-7.01 (m, 1H), 6.74-6.76 (m, 4H), 6.65-6.67 (m, 1H), 6.53-6.55 (m, 1H), 5.82 (bs, 1H), 4.84 (s, 2H), 2.68-2.81 (m, 2H), 1.80-1.82 (m, 2H), 1.65 (s, 6H), 1.52-1.54 (m, 1H), 1.25-1.26 (m, 3H).

### 5-[3-[[1-[(3-aminophenyl)-phenyl-methyl]sulfonyl-4-piperidyl]amino]phenyl]-3-(carboxymethoxy)-4-chloro-thiophene-2-carboxylic acid (A-34)

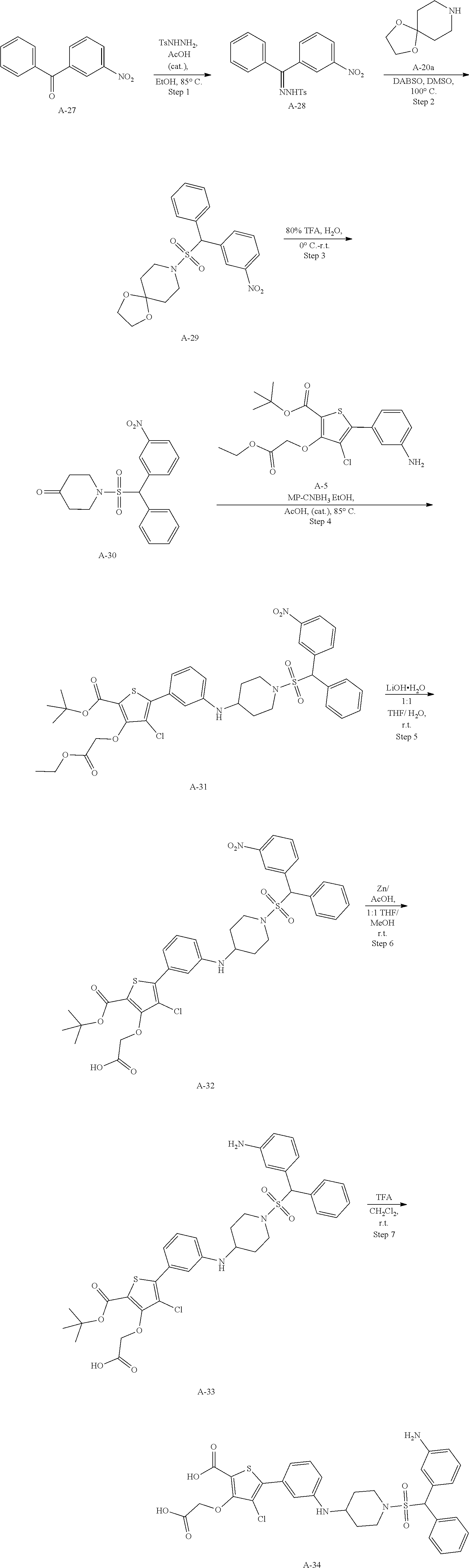

### Step 1: 4-methyl-N—[(Z)-[(3-nitrophenyl)-phenyl-methylene]amino]benzenesulfonamide (A-28)

Into a 100 mL sealed tube containing a well-stirred suspension of (4-nitrophenyl)-phenyl-methanone (A-27, 4 g, 17.60 mmol) in ethanol (6 mL) was added p-toluenesulfonyl hydrazide (3.93 g, 21.13 mmol, 2.81 mL) followed by acetic acid (105.71 mg, 1.76 mmol, 100.68 μL). The reaction mixture was heated at 85° C. for 16 h. The solvent was removed and the residue purified by silica-gel (230-400 mesh) flash column, eluting with 0-100% Ethyl acetate/petroleum ether to afford 4-methyl-N—[(Z)-[(3-nitrophenyl)-phenyl-methylene]amino]benzenesulfonamide (A-28, 4 g, 10.11 mmol, 57% yield) as an off-white solid. LCMS (ESI): m/z 396.0 [M+H]⁺. ¹H NMR (400 MHz, DMSO-d6): δ 8.24-8.20 (m, 1H), 7.92-7.88 (m, 2H), 7.82-7.75 (m, 2H), 7.61-7.59 (m, 3H), 7.52-7.48 (m, 1H), 7.47-7.32 (m, 4H), 7.20-7.13 (m, 1H), 2.48 (s, 3H).

### Step 2: 8-[(3-nitrophenyl)-phenyl-methyl]sulfonyl-1,4-dioxa-8-azaspiro[4.5]decane (A-29)

Into a 250 mL sealed tube containing a well-stirred solution of 4-methyl-N—[(Z)-[(3-nitrophenyl)-phenyl-methylene]amino]benzenesulfonamide (A-28, 4 g, 10.12 mmol) in anhydrous DMSO (40 mL) were added 1,4-dioxa-8-azaspiro[4.5]decane (A-20a, 1.45 g, 10.12 mmol, 1.29 mL) and 1,4-Diazabicyclo[2.2.2]octane-1,4-diium-1,4-disulfinate (DABSO) (1.34 g, 5.56 mmol). The reaction mixture was heated to 100° C. for 16 h. The reaction mixture was poured into 50% brine solution (250 mL) and extracted with Ethyl acetate (3×150 mL). Organic layers were combined, dried over anhydrous sodium sulfate, filtered and concentrated under reduced pressure. The residue was purified by flash column chromatography (50 g-Biotage column, prepacked with 230-400 silica gel using Isolera), eluting with 0-100% Ethyl acetate/petroleum ether to afford 8-[(3-nitrophenyl)-phenyl-methyl]sulfonyl-1,4-dioxa-8-azaspiro[4.5]decane (A-29, 2 g, 4.78 mmol, 47% yield) as an off-white solid. ¹H NMR (400 MHz, DMSO-d₆): δ 8.57 (s, 1H), 8.24 (d, J=11.2 Hz, 1H), 8.16 (d, J=10.4 Hz, 1H), 7.74 (t, J=22 Hz, 3H), 7.48-7.39 (m, 3H), 6.26 (s, 1H), 3.81 (s, 4H), 3.10-3.01 (m, 4H), 1.46-1.35 (m, 4H).

### Step 3: 1-[(3-nitrophenyl)-phenyl-methyl]sulfonylpiperidin-4-one (A-30)

Into a 100 mL single neck round bottom flask containing 8-[(3-nitrophenyl)-phenyl-methyl]sulfonyl-1,4-dioxa-8-azaspiro[4.5]decane (A-29, 2 g, 4.78 mmol) was added 4:1 TFA/water (11.84 g, 103.84 mmol, 10 mL) at 0° C. The reaction mixture was allowed to slowly come to room temperature and stirred for 3 h. The reaction mixture was carefully poured into ice cold saturated sodium bicarbonate solution. The aqueous layer was extracted with Ethyl acetate (3×100 mL). Organic layers were combined, washed with brine (50 mL), dried over sodium sulfate and filtered. The filtrate was concentrated under reduced pressure and the residue purified by silica gel flash column (50 g-Biotage column, prepacked with 230-400 silica gel using Isolera), eluting with 0-100% Ethyl acetate/petroleum ether to afford 1-[(3-nitrophenyl)-phenyl-methyl]sulfonylpiperidin-4-one (A-30, 1.5 g, 4.01 mmol, 84% yield) as an off-white solid. LCMS (ESI): m/z 373.0 [M−H]⁻. ¹H NMR (400 MHz, DMSO-d6): δ 8.49 (t, J=4 Hz, 1H), 8.24 (m, 1H), 8.07 (d, J=7.6 Hz, 1H), 7.66-7.60 (m, 3H), 7.49-7.44 (m, 3H), 5.45 (s, 1H), 3.50-3.40 (m, 2H), 3.32-3.31 (m, 2H), 2.45-2.29 (m, 4H).

### Step 4: tert-butyl 4-chloro-3-(2-ethoxy-2-oxo-ethoxy)-5-[3-[[1-[(3-nitrophenyl)-phenyl-methyl]sulfonyl-4-piperidyl]amino]phenyl]thiophene-2-carboxylate (A-31)

tert-butyl 4-chloro-3-(2-ethoxy-2-oxo-ethoxy)-5-[3-[[1-[(3-nitrophenyl)-phenyl-methyl]sulfonyl-4-piperidyl]amino]phenyl]thiophene-2-carboxylate (A-31, 600 mg, 778.91 μmol, 58% yield) was synthesized from 1-[(3-nitrophenyl)-phenyl-methyl]sulfonylpiperidin-4-one (A-30) and tert-butyl 5-(3-aminophenyl)-4-chloro-3-(2-ethoxy-2-oxo-ethoxy)thiophene-2-carboxylate (A-5) in a similar fashion to Compound A-23, except using 10 eq. MP-cyanoborohydride. ¹H NMR (400 MHz, DMSO-d₆): δ 8.49 (s, 1H), 8.23 (m, 1H), 8.09 (d, J=8 Hz, 1H), 7.67 (d, J=7.6 Hz, 2H), 7.61 (t, J=16 Hz, 1H), 7.51-7.45 (m, 3H), 7.23 (t, J=16 Hz, 1H), 6.98 (d, J=7.6 Hz, 1H), 6.83 (s, 1H), 6.59 (dd, J=8, 1.2 Hz, 1H), 5.41 (s, 1H), 4.90 (s, 2H), 4.31 (q, J=21.2 Hz, 2H), 3.65-3.49 (m, 3H), 3.46-3.35 (m, 3H), 2.87 (m, 1H), 2.50 (m, 1H), 2.11-1.91 (m, 2H), 1.38-1.30 (m, 12H).

### Step 5: 2-[[2-tert-butoxycarbonyl-4-chloro-5-[3-[[1-[(3-nitrophenyl)-phenyl-methyl]sulfonyl-4-piperidyl]amino]phenyl]-3-thienyl]oxy]acetic acid (A-32)

2-[[2-tert-butoxycarbonyl-4-chloro-5-[3-[[1-[(3-nitrophenyl)-phenyl-methyl]sulfonyl-4-piperidyl]amino]phenyl]-3-thienyl]oxy]acetic acid (A-32, 400 mg, 0.490 mmol, crude) was synthesized from tert-butyl 4-chloro-3-(2-ethoxy-2-oxo-ethoxy)-5-[3-[[1-[(3-nitrophenyl)-phenyl-methyl]sulfonyl-4-piperidyl]amino]phenyl]thiophene-2-carboxylate (A-31) in a similar fashion to Compound A-9, except using 2 eq. LiOH monohydrate. Upon completion, the aqueous layer was acidified with 1.5 N HCl, diluted with water and extracted with Ethyl acetate. Organic layer was separated, and aqueous layer was extracted with Ethyl acetate (2×). Organic layers were combined and washed with brine, dried over anhydrous Sodium sulfate and filtered. The filtrate was concentrated under reduced pressure and the residue triturated with diethyl ether (2×). The material was taken to the next step without further purification. LCMS (ESI): m/z 740.0 [M−H]⁻. ¹H NMR (400 MHz, DMSO-d₆): δ 8.57 (s, 1H), 8.25-8.16 (m, 2H), 7.77-7.70 (m, 3H), 7.48-7.39 (m, 3H), 7.21-7.15 (m, 1H), 6.81 (t, J=20 Hz, 2H), 6.66 (d, J=10.8 Hz, 1H), 6.25 (s, 1H), 4.84 (s, 2H), 3.17 (s, 2H), 1.82-1.73 (m, 2H), 1.52 (s, 9H), 1.27-1.24 (m, 7H).

### Step 6: 2-[[5-[3-[[1-[(3-aminophenyl)-phenyl-methyl]sulfonyl-4-piperidyl]amino]phenyl]-2-tert-butoxycarbonyl-4-chloro-3-thienyl]oxy]acetic acid (A-33)

2-[[5-[3-[[1-[(3-aminophenyl)-phenyl-methyl]sulfonyl-4-piperidyl]amino]phenyl]-2-tert-butoxycarbonyl-4-chloro-3-thienyl]oxy]acetic acid (A-33, 400 mg, 0.444 mmol, crude) was synthesized from 2-[[2-tert-butoxycarbonyl-4-chloro-5-[3-[[1-[(3-nitrophenyl)-phenyl-methyl]sulfonyl-4-piperidyl]amino]phenyl]-3-thienyl]oxy]acetic acid (A-32) in a similar fashion to Compound A-10, except upon completion the reaction mixture was filtered through Celite, washing with methanol and the filtrate was evaporated under reduced pressure. The residue was triturated with diethyl ether and the material was used in the next step without further purification. LCMS (ESD): m/z 712.1 [M+H]⁺, ¹H NMR (400 MHz, DMSO-d₆): δ 7.67 (d, J=9.6 Hz, 2H), 7.00 (m, 4H), 7.19-7.14 (m, 1H), 7.04-6.78 (m, 5H), 6.58 (m, 2H), 5.79 (d, J=10 Hz, 1H), 5.60 (s, 1H), 5.18 (s, 2H), 5.18 (s, 2H), 3.52-3.49 (m, 2H), 2.73-2.62 (m, 3H), 1.82-1.78 (m, 4H), 1.36 (s, 9H).

### Step 7:5-[3-[[1-[(3-aminophenyl)-phenyl-methyl]sulfonyl-4-piperidyl]amino]phenyl]-3-(carboxymethoxy)-4-chloro-thiophene-2-carboxylic acid (A-34)

5-[3-[[1-[(3-aminophenyl)-phenyl-methyl]sulfonyl-4-piperidyl]amino]phenyl]-3-(carboxymethoxy)-4-chloro-thiophene-2-carboxylic acid (A-34, 200 mg, 294.74 μmol, 52% yield) was synthesized from 2-[[5-[3-[[1-[(3-aminophenyl)-phenyl-methyl]sulfonyl-4-piperidyl]amino]phenyl]-2-tert-butoxycarbonyl-4-chloro-3-thienyl]oxy]acetic acid (A-33) in a similar fashion to Compound A-26, except using 30 eq. TFA. The product was purified by reverse phase chromatography. LCMS (ESI): m/z 655 [M−H]⁻. ¹H NMR (400 MHz, DMSO-d₆): δ 7.69 (d, J=9.20 Hz, 2H), 7.46-7.34 (m, 6H), 7.19 (t, J=20.8 Hz, 1H), 7.07 (d, J=9.2 Hz, 1H), 6.82 (t, J=18 Hz, 2H), 6.67 (d, J=10.8 Hz, 1H), 5.90 (s, 1H), 4.93 (s, 2H), 3.51 (s, 2H), 3.27 (m, 2H), 2.73-2.57 (m, 4H), 2.08 (s, 2H), 1.82-1.78 (m, 2H), 1.18-1.16 (m, 2H).

### 5-(3-((1-((3-Aminobenzyl)sulfonyl)piperidin-4-yl)oxy)phenyl)-3-(carboxymethoxy)-4-chlorothiophene-2-carboxylic acid (A-43)

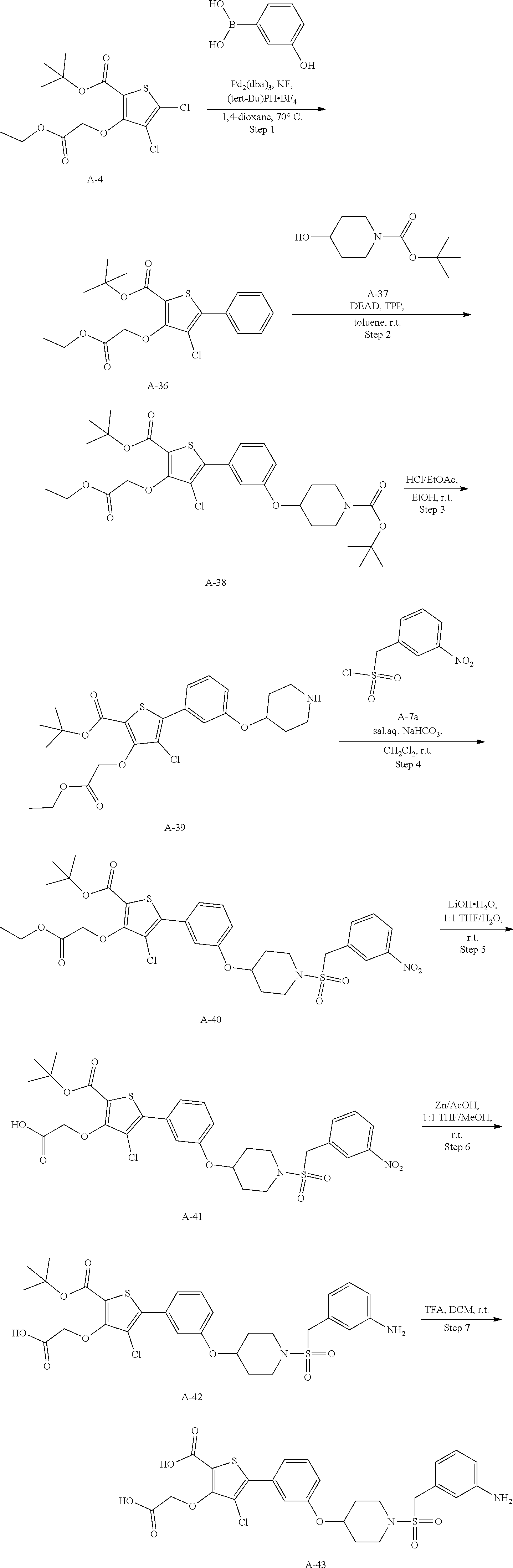

### Step 1: tert-butyl 4-chloro-3-(2-ethoxy-2-oxo-ethoxy)-5-(3-hydroxyphenyl)thiophene-2-carboxylate (A-36)

tert-butyl 4-chloro-3-(2-ethoxy-2-oxo-ethoxy)-5-(3-hydroxyphenyl)thiophene-2-carboxylate (A-36, 1.4 g, 2.71 mmol, 60% yield) was prepared from tert-butyl 4,5-dichloro-3-(2-ethoxy-2-oxo-ethoxy)thiophene-2-carboxy late (A-4) and (3-hydroxyphenyl)boronic acid (A-35) in a similar fashion to Compound A-5, except using 1.5 eq. A-35. LCMS (ESI): m/z 411.0 [M−H]⁻.

### Step 2: tert-butyl 4-[3-[5-tert-butoxycarbonyl-3-chloro-4-(2-ethoxy-2-oxo-ethoxy)-2-thienyl]phenoxy]piperidine-1-carboxylate (A-38)

Into a 50 mL single neck round bottom flask containing a well-stirred solution of tert-butyl 4-chloro-3-(2-ethoxy-2-oxo-ethoxy)-5-(3-hydroxyphenyl)thiophene-2-carboxylate (A-36, 1.4 g, 3.39 mmol) and tert-butyl 4-hydroxypiperidine-1-carboxylate (A-37, 2.05 g, 10.17 mmol) in anhydrous toluene (15 mL) were added triphenylphosphine (2.67 g, 10.17 mmol) and diethylazodicarboxylate (1.77 g, 10.17 mmol). After 16 h, the solvent was removed under reduced pressure and the residue purified by silica gel flash column (50 g-Biotage column, prepacked with 230-400 silica gel using Isolera), eluting with 0-100% Ethyl acetate/pet ether to afford tert-butyl 4-[3-[5-tert-butoxycarbonyl-3-chloro-4-(2-ethoxy-2-oxo-ethoxy)-2-thienyl]phenoxy]piperidine-1-carboxylate (A-38, 1.2 g, 1.88 mmol, 56% yield) as a pale-yellow solid. LCMS (ESI): m/z 484.0 [M−2 tBu+H]⁺. ¹H NMR (400 MHz, DMSO-d₆): δ 7.39-7.35 (m, 1H), 7.25 (d, J=2.0 Hz, 2H), 6.98 (dd, J=8.0, 1.6 Hz, 1H), 4.92 (s, 2H), 4.57-4.51 (m, 2H), 4.33-4.28 (m, 1H), 3.76-3.70 (m, 2H), 3.42-3.35 (m, 2H), 1.98-1.95 (m, 2H), 1.84-1.78 (m, 2H), 1.58 (s, 9H), 1.48 (s, 9H), 1.33 (t, J=7.2 Hz, 3H).

### Step 3: tert-butyl 4-chloro-3-(2-ethoxy-2-oxo-ethoxy)-5-[3-(4-piperidyloxy)phenyl]thiophene-2-carboxylate (A-39)

Into a 50 mL single neck round bottom flask containing a well-stirred solution of tert-butyl 4-[3-[5-tert-butoxycarbonyl-3-chloro-4-(2-ethoxy-2-oxo-ethoxy)-2-thienyl]phenoxy]piperidine-1-carboxylate (A-38, 1.2 g, 2.01 mmol) in ethanol (10 mL) was added hydrogen chloride (1M in Ethyl acetate) (8.00 g, 219.41 mmol, 10 mL) at 0° C. The resulting mixture was stirred at room temperature for 5 h. The crude reaction mixture was concentrated and azeotroped with toluene (2×20 mL). The residue was triturated with diethyl ether (10 mL) to afford tert-butyl 4-chloro-3-(2-ethoxy-2-oxo-ethoxy)-5-[3-(4-piperidyloxy)phenyl]thiophene-2-carboxylate (A-39, 800 mg, 1.38 mmol, 69% yield, HCl salt) as an off-white solid. LCMS (ESI): m/z 495.9 [M+H]⁺. ¹H NMR (400 MHz, DMSO-d₆): δ 8.51 (d, J=9.6 Hz, 2H), 7.49-7.46 (m, 1H), 7.29-7.27 (m, 2H), 7.16 (d, J=11.2 Hz, 1H), 4.95 (s, 2H), 4.75-4.73 (m, 2H), 4.22-4.14 (m, 5H), 3.14-3.12 (m, 4H), 1.52 (s, 9H), 1.22 (t, J=18.4 Hz, 3H).

### Step 4: tert-butyl 4-chloro-3-(2-ethoxy-2-oxo-ethoxy)-5-[3-[[1-[(3-nitrophenyl)methylsulfonyl]-4-piperidyl]oxy]phenyl]thiophene-2-carboxylate (A-40)

tert-butyl 4-chloro-3-(2-ethoxy-2-oxo-ethoxy)-5-[3-[[1-[(3-nitrophenyl)methylsulfonyl]-4-piperidyl]oxy]phenyl]thiophene-2-carboxylate (A-40, 600 mg, 0.7336 mmol, 49% yield) was synthesized from tert-butyl 4-chloro-3-(2-ethoxy-2-oxo-ethoxy)-5-[3-(4-piperidyloxy)phenyl]thiophene-2-carboxylate hydrochloride (A-39) and (3-nitrophenyl)methane sulfonyl chloride (A-7a) in a similar fashion to Compound A-8, except using 1.5 eq. A-39. LCMS (ESI): 638.08 m/z [M−tBu+H]⁺.

### Step 5: 2-[[5-[3-[[1-[(3-aminophenyl)methylsulfonyl]-4-piperidyl]oxy]phenyl]-2-tert-butoxycarbonyl-4-chloro-3-thienyl]oxy]acetic acid (A-41)

Into a 50 mL single neck round bottom flask containing a well-stirred solution of tert-butyl 4-chloro-3-(2-ethoxy-2-oxo-ethoxy)-5-[3-[[1-[(3-nitrophenyl)methylsulfonyl]-4-piperidyl]oxy]phenyl]thiophene-2-carboxylate (A-40, 600 mg, 0.89935 mmol) in a mixture of tetrahydrofuran and water (1:1) (12 mL) was added LiOH (64.61 mg, 2.70 mmol). The resulting mixture was stirred for 3 h. The reaction mixture was cooled to 10° C., acidified with 0.5 M aqueous HCl and the aqueous phase extracted with ethyl acetate (2×50 mL). The organic phases were dried over sodium sulfate, filtered and concentrated under reduced pressure. The residue was washed with ethyl acetate to afford 2-[[5-[3-[[1-[(3-aminophenyl)methylsulfonyl]-4-piperidyl]oxy]phenyl]-2-tert-butoxycarbonyl-4-chloro-3-thienyl]oxy]acetic acid (A-41, 350 mg, 0.54 mmol, 60% yield) as an off-white solid. The material was used in the next step without further purification. LCMS (ESI): m/z 665.0 [M−H]⁻. ¹H NMR (400 MHz, DMSO-d₆): δ 8.33 (s, 1H), 8.24-8.23 (m, 1H), 7.90-7.88 (m, 1H), 7.71 (m, 1H), 7.45-7.40 (m, 1H), 7.23 (m, 2H), 7.08 (m, 1H), 4.69-4.67 (m, 3H), 4.37 (s, 2H), 3.22 (m, 4H), 1.98-1.97 (m, 4H), 1.50 (s, 9H).

### Step 6: 2-[[5-[3-[[1-[(3-aminophenyl)methylsulfonyl]-4-piperidyl]oxy]phenyl]-2-tert-butoxycarbonyl-4-chloro-3-thienyl]oxy]acetic acid (A-42)

2-[[5-[3-[[1-[(3-aminophenyl)methylsulfonyl]-4-piperidyl]oxy]phenyl]-2-tert-butoxycarbonyl-4-chloro-3-thienyl]oxy]acetic acid (A-42, 150 mg, 0.219 mmol, 42% yield) was synthesized from 2-[[2-tert-butoxycarbonyl-4-chloro-5-[3-[[1-[(3-nitrophenyl)methylsulfonyl]-4-piperidyl]oxy]phenyl]-3-thienyl]oxy]acetic acid (A-41) in a similar fashion to Compound A-10, except using 3 eq. Zinc. Upon completion, the reaction mixture was concentrated, and the residue purified by reverse phase column chromatography. LCMS (ESI): m/z 635.0 [M−H]⁻.

### Step 7: 5-[3-[[1-[(3-aminophenyl)methylsulfonyl]-4-piperidyl]oxy]phenyl]-3-(carboxymethoxy)-4-chloro-thiophene-2-carboxylic acid (A-43)

5-[3-[[1-[(3-aminophenyl)methylsulfonyl]-4-piperidyl]oxy]phenyl]-3-(carboxymethoxy)-4-chloro-thiophene-2-carboxylic acid (A-43, 80 mg, crude) was prepared from 2-[[5-[3-[[1-[(3-aminophenyl)methylsulfonyl]-4-piperidyl]oxy]phenyl]-2-tert-butoxycarbonyl-4-chloro-3-thienyl]oxy]acetic acid (A-42) in a similar fashion to Compound A-26, except using 20 eq. TFA. LCMS (ESI): m/z 581.0 [M+H]⁺.

### 2-((5-(3-(N-(1-((3-Aminobenzyl)sulfonyl)piperidin-4-yl)benzamido)phenyl)-2-(tert-butoxycarbonyl)-4-chlorothiophen-3-yl)oxy)acetic acid (A-46)

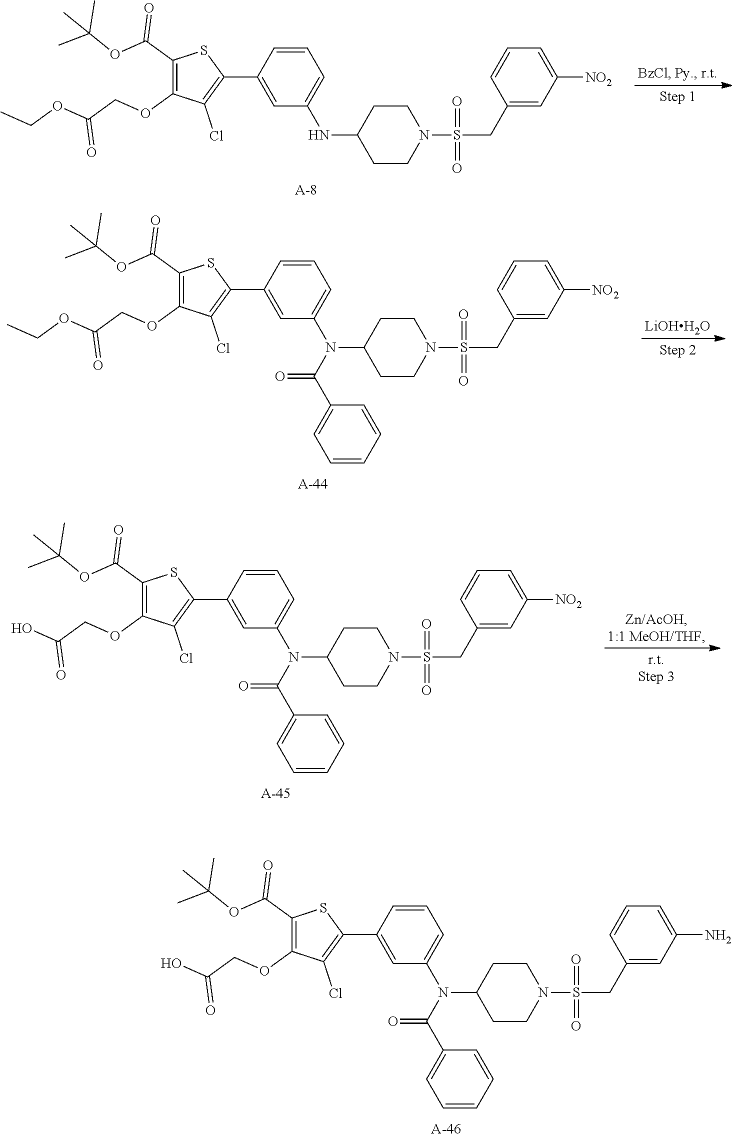

### Step 1: tert-butyl 5-[3-[benzoyl-[1-[(3-nitrophenyl)methylsulfonyl]-4-piperidyl]amino]phenyl]-4-chloro-3-(2-ethoxy-2-oxo-ethoxy)thiophene-2-carboxylate (A-44)

Into a 25 mL single neck round bottom flask containing a well-stirred solution of tert-butyl 4-chloro-3-(2-ethoxy-2-oxo-ethoxy)-5-[3-[[1-[(3-nitrophenyl)methylsulfonyl]-4-piperidyl]amino]phenyl]thiophene-2-carboxylate (A-8, 220 mg, 316.91 μmol) in anhydrous dichloromethane (10 mL) were added benzoyl chloride (44.55 mg, 316.91 μmol) and triethylamine (32.07 mg, 316.91 μmol, 44.17 μL). After 4 h the reaction mixture was concentrated under reduced pressure and the residue purified by Isolera (230-400 mesh silica-gel column), eluting with 30-40% Ethyl acetate/petroleum ether to afford tert-butyl 5-[3-[benzoyl-[1-[(3-nitrophenyl)methylsulfonyl]-4-piperidyl]amino]phenyl]-4-chloro-3-(2-ethoxy-2-oxo-ethoxy)thiophene-2-carboxylate (A-44, 270 mg, 43.97 μmol, 14% yield) as a yellow foam. LCMS (ESI): m/z 798.32 [M+H]⁺.

### Step 2: 2-((2-(tert-butoxycarbonyl)-4-chloro-5-(3-(N-(1-((3-nitrobenzyl)sulfonyl)piperidin-4-yl)benzamido)phenyl)thiophen-3-yl)oxy)acetic acid (A-45)

2-((2-(tert-butoxycarbonyl)-4-chloro-5-(3-(N-(1-((3-nitrobenzyl)sulfonyl)piperidin-4-yl)benzamido)phenyl)thiophen-3-yl)oxy)acetic acid (A-45, 0.266 g, crude) was synthesized from tert-butyl 5-[3-[benzoyl-[1-[(3-nitrophenyl)methylsulfonyl]-4-piperidyl]amino]phenyl]-4-chloro-3-(2-ethoxy-2-oxo-ethoxy)thiophene-2-carboxylate (A-44) in a similar fashion to Compound A-9 except using one eq. LiOH monohydrate. Upon completion, the reaction mixture was concentrated under reduced pressure. The aqueous layer was acidified with 1.5 N HCl and diluted with Ethyl acetate and water. The layers were separated, and the aqueous layer extracted with Ethyl acetate (2×). Combined organic phases were washed with brine and dried over anhydrous Sodium sulfate and filtered. The filtrate was concentrated under reduced pressure. The material was used in the next step without purification. LCMS (ESI): m/z 770.27 [M+H]⁺.

### Step 3: 2-((5-(3-(N-(1-((3-Aminobenzyl)sulfonyl)piperidin-4-yl)benzamido)phenyl)-2-(tert-butoxycarbonyl)-4-chlorothiophen-3-yl)oxy)acetic acid (A-46)

Into a 50 mL single neck round bottom flask containing a well-stirred solution of 2-((2-(tert-butoxycarbonyl)-4-chloro-5-(3-(N-(1-((3-nitrobenzyl)sulfonyl)piperidin-4-yl)benzamido)phenyl)thiophen-3-yl)oxy)acetic acid (A-45, 266.77 mg, 346.34 μmol) in a mixture of methanol and tetrahydrofuran (1:1) (6 mL) was added Zinc dust (50.40 mg, 770.73 μmol). The reaction mixture was cooled to 0° C. and acetic acid (20.80 mg, 346.34 μmol, 19.81 μL) was added. The reaction mixture was stirred at rt for 3 h. The reaction mixture was filtered through Celite, washing with methanol. The filtrate was evaporated under reduced pressure at 35° C. and the residue purified by reverse phase chromatography on a SunFire C₁₈ column (19×150 mm; with solvent A: 0.1% formic acid in water and solvent B: acetonitrile; Flow rate: 15 mL/min; RT=15.08 min to obtain 2-((5-(3-(N-(1-((3-Aminobenzyl)sulfonyl)piperidin-4-yl)benzamido)phenyl)-2-(tert-butoxycarbonyl)-4-chlorothiophen-3-yloxy)acetic acid (A-46, 170 mg, 195.20 μmol, 56% yield) as a pale yellow foam. LCMS (ESI): m/z 740.28 [M+H]⁺.

### 2-((5-(3-(N-(1-((3-Aminobenzyl)sulfonyl)-2,2-dimethylpiperidin-4-yl)benzamido)phenyl)-2-(tert-butoxycarbonyl)-4-chlorothiophen-3-yl)oxy)acetic acid (A-50)

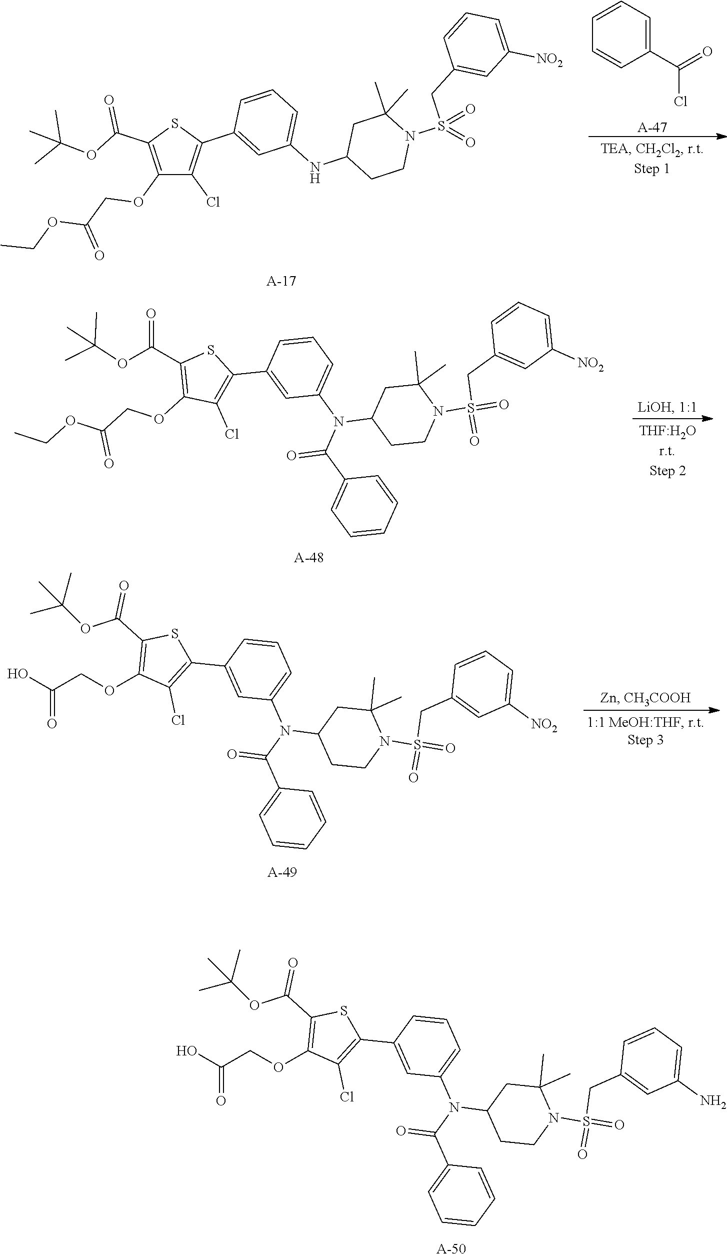

### Step 1: tert-butyl 5-[3-[benzoyl-[2,2-dimethyl-1-[(3-nitrophenyl)methylsulfonyl]-4-piperidyl]amino]phenyl]-4-chloro-3-(2-ethoxy-2-oxo-ethoxy)thiophene-2-carboxylate (A-48)

Into a 25 mL single neck round bottom flask containing a well-stirred solution of tert-butyl 4-chloro-5-[3-[[2,2-dimethyl-1-[(3-nitrophenyl)methylsulfonyl]-4-piperidyl]amino]phenyl]-3-(2-ethoxy-2-oxo-ethoxy)thiophene-2-carboxylate (A-17, 300 mg, 315.80 μmol) in dichloromethane (3 mL) were added triethylamine (95.87 mg, 947.39 μmol, 132.05 μL) and benzoyl chloride (A-47, 66.59 mg, 473.70 μmol) at 0° C. under nitrogen atmosphere. The resulting mixture was stirred at room temperature for 4 h. The reaction mixture was diluted with water (10 mL) and extracted with dichloromethane (2×10 mL). The combined organic layers were dried over anhydrous sodium sulfate, filtered and concentrated under reduced pressure. The residue was purified by silica-gel column (100-200 mesh) to afford tert-butyl 5-[3-[benzoyl-[2,2-dimethyl-1-[(3-nitrophenyl)methylsulfonyl]-4-piperidyl]amino]phenyl]-4-chloro-3-(2-ethoxy-2-oxo-ethoxy)thiophene-2-carboxylate (A-48, 240 mg, 244.42 μmol, 77% yield) as a brown gummy liquid. LCMS (ESI): m/z 826.1 [M+H]⁺.

### Step 2: 2-[[5-[3-[benzoyl-[2,2-dimethyl-1-[(3-nitrophenyl)methylsulfonyl]-4-piperidyl]amino]phenyl]-2-tert-butoxycarbonyl-4-chloro-3-thienyl]oxy]acetic acid (A-49)

2-[[5-[3-[benzoyl-[2,2-dimethyl-1-[(3-nitrophenyl)methylsulfonyl]-4-piperidyl]amino]phenyl]-2-tert-butoxycarbonyl-4-chloro-3-thienyl]oxy]acetic acid (A-49, 150 mg, crude) was synthesized from tert-butyl 5-[3-[benzoyl-[2,2-dimethyl-1-[(3-nitrophenyl)methylsulfonyl]-4-piperidyl]amino]phenyl]-4-chloro-3-(2-ethoxy-2-oxo-ethoxy)thiophene-2-carboxylate (A-48) in a similar fashion to Compound A-9, except a 1:1 ratio of water/THF was used, and upon completion the reaction mixture was diluted with water and extracted with ethyl acetate (2×). The combined organic layers were dried over anhydrous sodium sulfate, filtered and concentrated under reduced pressure. The residue was washed with pentane. The material was used in the next step without further purification. LCMS (ESI): m/z 796.0 [M+H]⁺.

### Step 3: 2-((5-(3-(N-(1-((3-Aminobenzyl)sulfonyl)-2,2-dimethylpiperidin-4-yl)benzamido)phenyl)-2-(tert-butoxycarbonyl)-4-chlorothiophen-3-yl)oxy)acetic acid (A-50)

2-((5-(3-(N-(1-((3-Aminobenzyl)sulfonyl)-2,2-dimethylpiperidin-4-yl)benzamido)phenyl)-2-(tert-butoxycarbonyl)-4-chlorothiophen-3-yl)oxy)acetic acid (A-50, 80 mg, 102.57 μmol, 68% yield) was synthesized from 2-[[5-[3-[benzoyl-[2,2-dimethyl-1-[(3-nitrophenyl)methylsulfonyl]-4-piperidyl]amino]phenyl]-2-tert-butoxycarbonyl-4-chloro-3-thienyl]oxy]acetic acid (A-49) in a similar fashion to Compound A-10, except using 10 eq. Zinc powder and 5 eq. Acetic acid. Upon completion, the reaction mixture was filtered through Celite, washing with methanol. The solvent was removed, and the residue purified by reverse phase chromatography. LCMS (ESI): m/z 766.1 [M−H]⁻. ¹H NMR (400 MHz, DMSO-d6): $7.52-7.47 (m, 2H), 7.37 (s, 1H), 7.29-7.19 (m, 6H), 6.59-6.57 (m, 1H), 6.52 (s, 1H), 6.37 (d, J=8.04 Hz, 1H), 6.26 (d, J=7.24 Hz, 1H), 4.84 (s, 2H), 4.19-4.01 (m, 3H), 3.61-3.59 (m, 1H), 3.34-3.32 (m, 2H), 1.78-1.75 (m, 3H), 1.52 (s, 9H), 1.47-1.37 (m, 7H), 1.19-1.16 (t, J=7.12 Hz, 1H).

### 2-((5-(3-(N-(1-((3-Aminobenzyl)sulfonyl)piperidin-4-yl) isobutyramido)phenyl)-2-(tert-butoxycarbonyl)-4-chlorothiophen-3-yl)oxy)acetic acid (A-54)

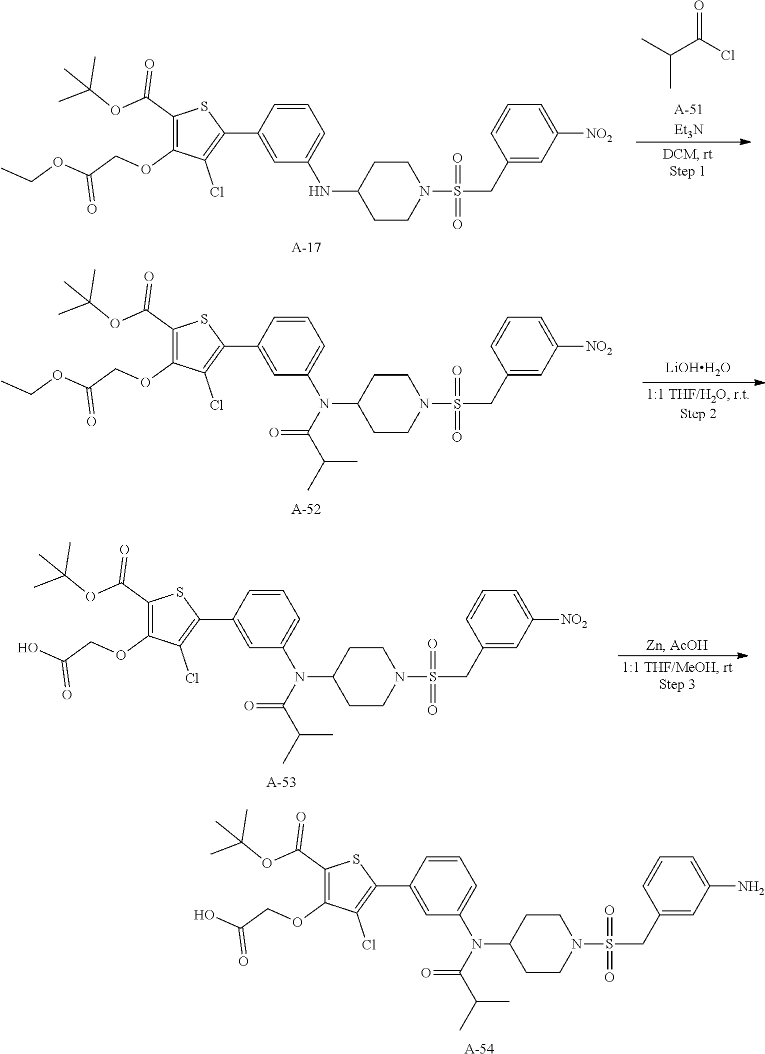

### Step 1: tert-butyl-4-chloro-3-(2-ethoxy-2-oxo-ethoxy)-5-[3-[2-methylpropanoyl-[1-[(3-nitrophenyl)methylsulfonyl]-4-piperidyl]amino]phenyl]thiophene-2-carboxylate (A-52)

tert-butyl-4-chloro-3-(2-ethoxy-2-oxo-ethoxy)-5-[3-[2-methylpropanoyl-[1-[(3-nitrophenyl)methylsulfonyl]-4-piperidyl]amino]phenyl]thiophene-2-carboxylate (A-52, 240 mg, 159.02 μmol, 32% yield) was synthesized from tert-butyl-4-chloro-3-(2-ethoxy-2-oxo-ethoxy)-5-[3-[[1-[(3-nitrophenyl)methylsulfonyl]-4-piperidyl]amino]phenyl]thiophene-2-carboxylate (A-17) and isobutyryl chloride (A-51) in a similar fashion to Compound A-48. LCMS (ESI): m/z 708 [M−tBu+H]⁺.

### Step 2: 2-[[2-tert-butoxycarbonyl-4-chloro-5-[3-[2-methylpropanoyl-[1-[(3-nitrophenyl)methylsulfonyl]-4-piperidyl]amino]phenyl]-3-thienyl]oxy]acetic acid (A-53)

2-[[2-tert-butoxycarbonyl-4-chloro-5-[3-[2-methylpropanoyl-[1-[(3-nitrophenyl)methylsulfonyl]-4-piperidyl]amino]phenyl]-3-thienyl]oxy]acetic acid (A-53, 180 mg, crude) was synthesized from tert-butyl-4-chloro-3-(2-ethoxy-2-oxo-ethoxy)-5-[3-[2-methylpropanoyl-[1-[(3-nitrophenyl)methylsulfonyl]-4-piperidyl]amino]phenyl]thiophene-2-carboxylate (A-52) in a similar fashion to Compound A-9, except upon completion water was added and the resulting solution was acidified with 1.5 N HCl and the aqueous phase was extracted with Ethyl acetate. The organic layers were dried over anhydrous Sodium sulfate, filtered and concentrated under reduced pressure. The material was used in the next step without purification. LCMS (ESI): m/z 734 [M−H]⁻.

### Step 3: 2-((5-(3-(N-(1-((3-Aminobenzyl)sulfonyl)piperidin-4-yl) isobutyramido)phenyl)-2-(tert-butoxycarbonyl)-4-chlorothiophen-3-yl)oxy)acetic acid (A-54)

2-((5-(3-(N-(1-((3-Aminobenzyl)sulfonyl)piperidin-4-yl) isobutyramido)phenyl)-2-(tert-butoxycarbonyl)-4-chlorothiophen-3-yl)oxy)acetic acid (A-54, 100 mg, 112.11 μmol, 46% yield) was synthesized from 2-[[2-tert-butoxycarbonyl-4-chloro-5-[3-[2-methylpropanoyl-[1-[(3-nitrophenyl)methylsulfonyl]-4-piperidyl]amino]phenyl]-3-thienyl]oxy]acetic acid (A-53) in a similar fashion to Compound A-10, except using 5 eq. Zinc and 4 eq. Acetic acid. Upon completion, the reaction mixture was filtered through Celite and the solvent evaporated under reduced pressure. The residue was purified by reverse phase chromatography. LCMS (ESI): m/z 706 [M+H]⁺. ¹H NMR (400 MHz, DMSO-d₆): δ 7.76-7.71 (m, 1H), 7.69-7.62 (m, 1H), 7.54 (s, 1H), 7.42-7.35 (m, 1H), 7.11-6.93 (m, 1H), 6.72-6.63 (m, 3H), 4.85 (s, 2H), 4.52-4.48 (m, 2H), 4.21 (s, 2H), 3.56-3.53 (m, 4H), 2.80 (t, J=11.6 Hz, 4H), 1.52 (s, 9H), 0.92 (bs, 6H).

### 2-[[5-[3-[[1-[(3-aminophenyl)methylsulfonyl]-4-piperidyl]-methyl-amino]phenyl]-2-tert-butoxycarbonyl-4-chloro-3-thienyl]oxy]acetic acid (A-59)

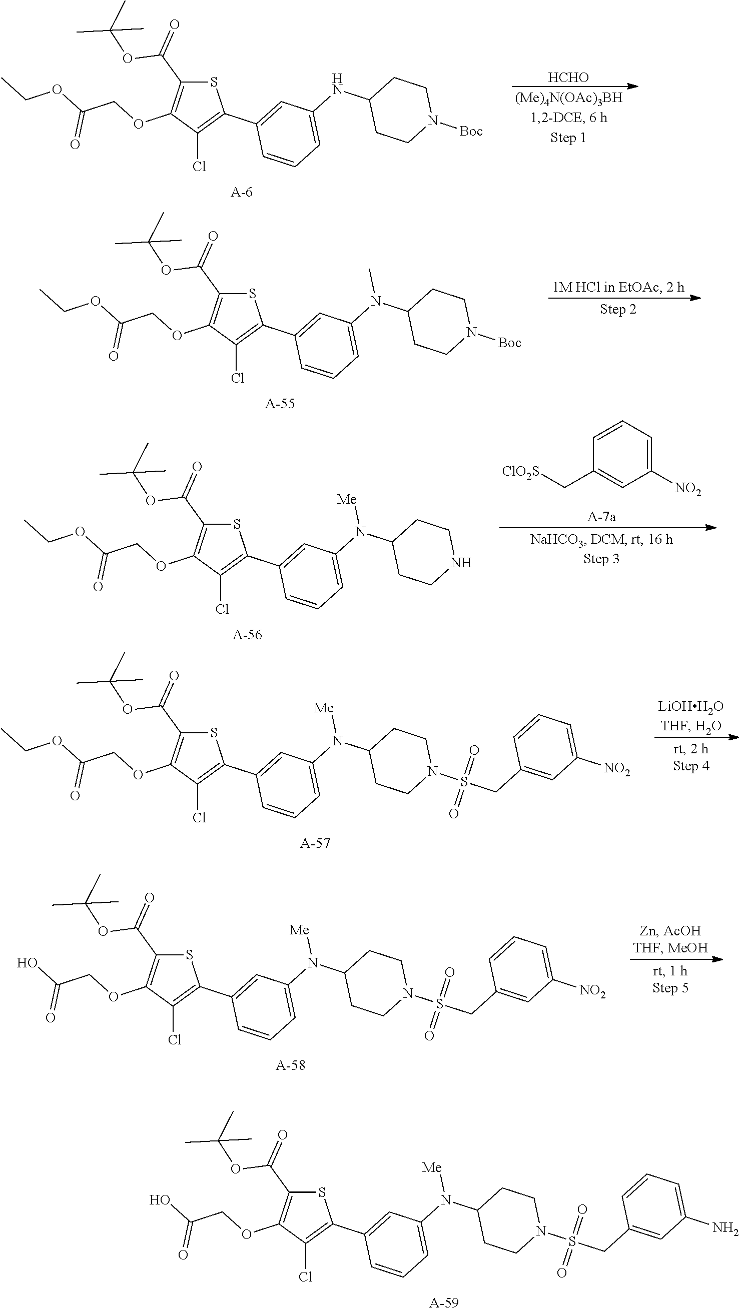

### Step 1: tert-butyl 4-[3-[5-tert-butoxycarbonyl-3-chloro-4-(2-ethoxy-2-oxo-ethoxy)-2-thienyl]-N-methyl-anilino]piperidine-1-carboxylate (A-55)

Into a 100 mL single neck round bottom flask containing a well-stirred solution of tert-butyl 4-[3-[5-tert-butoxycarbonyl-3-chloro-4-(2-ethoxy-2-oxo-ethoxy)-2-thienyl]anilino]piperidine-1-carboxylate (A-6, 600 mg, 1.01 mmol) in 1,2-dichloroethane (25 mL) was added formaldehyde solution (37 wt. % in water) (60.54 mg, 2.02 mmol) followed by tetramethylammonium triacetoxyborohydride (397.86 mg, 1.51 mmol). The reaction mixture was stirred at room temperature for 6 h. The mixture was diluted with water (25 mL) and extracted with ethyl acetate (2×50 mL), the combined organic layer was washed with brine (25 mL) and dried over anhydrous sodium sulfate. The solvent was removed under reduced pressure and the residue purified by flash silica gel (230-400 mesh) column chromatography (15-20% of Ethyl acetate in petroleum ether) to afford tert-butyl 4-[3-[5-tert-butoxycarbonyl-3-chloro-4-(2-ethoxy-2-oxo-ethoxy)-2-thienyl]-N-methyl-anilino]piperidine-1-carboxylate (A-55, 500 mg, 738.71 μmol, 73% yield) as a yellow solid. LCMS (ES+): m/z 497.1 [M−2 tBu+H]⁺.

### Step 2: tert-butyl-4-chloro-3-(2-ethoxy-2-oxo-ethoxy)-5-[3-[methyl (4-piperidyl)amino]phenyl]thiophene-2-carboxylate (A-56)

Into a 100 mL round bottom flask containing a well-stirred solution of tert-butyl 4-[3-[5-tert-butoxycarbonyl-3-chloro-4-(2-ethoxy-2-oxo-ethoxy)-2-thienyl]-N-methyl-anilino]piperidine-1-carboxy late (A-55, 0.500 g, 820.79 μmol) in ethanol (2 mL) was added 1M HCl in Ethyl acetate (820.79 μmol) dropwise at 0° C. The reaction mixture was stirred for 3 h at room temperature. The volatiles were removed under reduced pressure and the residue triturated with diethyl ether, filtered and concentrated under reduced pressure to afford tert-butyl 4-chloro-3-(2-ethoxy-2-oxo-ethoxy)-5-[3-[methyl (4-piperidyl)amino]phenyl]thiophene-2-carboxylate (A-56, 0.400 g, 593.93 μmol, 72% yield, HCl salt) as a yellow solid. The material was used in the next step without further purification. LCMS (ES+): m/z 509.2 [M+H]⁺.

### Step 3: tert-butyl 4-chloro-3-(2-ethoxy-2-oxo-ethoxy)-5-[3-[methyl-[1-[(3-nitrophenyl)methylsulfonyl]-4-piperidyl]amino]phenyl]thiophene-2-carboxylate (A-57)

Into a 100 mL round bottom flask containing a well-stirred solution of tert-butyl 4-chloro-3-(2-ethoxy-2-oxo-ethoxy)-5-[3-[methyl (4-piperidyl)amino]phenyl]thiophene-2-carboxylate (A-56, 0.400 g, 733.25 μmol) in dichloromethane (5 mL) was added an aqueous solution (10%) of sodium bicarbonate (20 mL). The reaction mixture was cooled to 0° C. Then, (3-nitrophenyl)methanesulfonyl chloride (A-7a, 172.79 mg, 733.25 μmol) was added. The reaction mixture was stirred at room temperature for 3 h. The reaction mixture was extracted with DCM (2×100 mL). The organic layer was washed with water (75 mL) and brine (25 mL) and dried over anhydrous sodium sulfate. The solvent was removed under reduced pressure and the residue purified by flash silica gel (230-400 mesh) column chromatography (25-30% of Ethyl acetate in petroleum ether) to afford tert-butyl 4-chloro-3-(2-ethoxy-2-oxo-ethoxy)-5-[3-[methyl-[1-[(3-nitrophenyl)methylsulfonyl]-4-piperidyl]amino]phenyl]thiophene-2-carboxylate (A-57, 0.480 g, 521.86 μmol, 71% yield) as a yellow solid. LCMS (ES+): m/z 709.1 [M+H]⁺.

### Step 4: 2-[[2-tert-butoxycarbonyl-4-chloro-5-[3-[methyl-[1-[(3-nitrophenyl)methylsulfonyl]-4-piperidyl]amino]phenyl]-3-thienyl]oxy]acetic acid (A-58)

2-[[2-tert-butoxycarbonyl-4-chloro-5-[3-[methyl-[1-[(3-nitrophenyl)methylsulfonyl]-4-piperidyl]amino]phenyl]-3-thienyl]oxy]acetic acid (A-58, 400 mg, 317.56 μmol, 47% yield) was synthesized from tert-butyl 4-chloro-3-(2-ethoxy-2-oxo-ethoxy)-5-[3-[methyl-[1-[(3-nitrophenyl)methylsulfonyl]-4-piperidyl]amino]phenyl]thiophene-2-carboxylate (A-57) in a similar fashion to Compound A-9, except using 1 eq. LiOH monohydrate. Upon completion, the reaction was acidified with 1.5 N HCl in water (pH 4-5) and extracted with Ethyl acetate (2×50 mL). The combined organic layer was dried over anhydrous sodium sulfate, filtered and solvent removed under reduced pressure. The material was used in the next step without further purification. LCMS (ES+): m/z 680.0 [M+H]⁺.

### Step 5: 2-[[5-[3-[[1-[(3-aminophenyl)methylsulfonyl]-4-piperidyl]-methyl-amino]phenyl]-2-tert-butoxycarbonyl-4-chloro-3-thienyl]oxy]acetic acid (A-59)

2-[[5-[3-[[1-[(3-aminophenyl)methylsulfonyl]-4-piperidyl]-methyl-amino]phenyl]-2-tert-butoxycarbonyl-4-chloro-3-thienyl]oxy]acetic acid (A-59, 0.250 g, 230.70 μmol, 39% yield, Formic acid salt) was synthesized from 2-[[2-tert-butoxycarbonyl-4-chloro-5-[3-[methyl-[1-[(3-nitrophenyl)methylsulfonyl]-4-piperidyl]amino]phenyl]-3-thienyl]oxy]acetic acid (A-58) in a similar fashion to Compound A-10, except using 5 eq. Acetic acid. LCMS (ES+): m/z 650.2 [M+H]⁺.

### 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62)

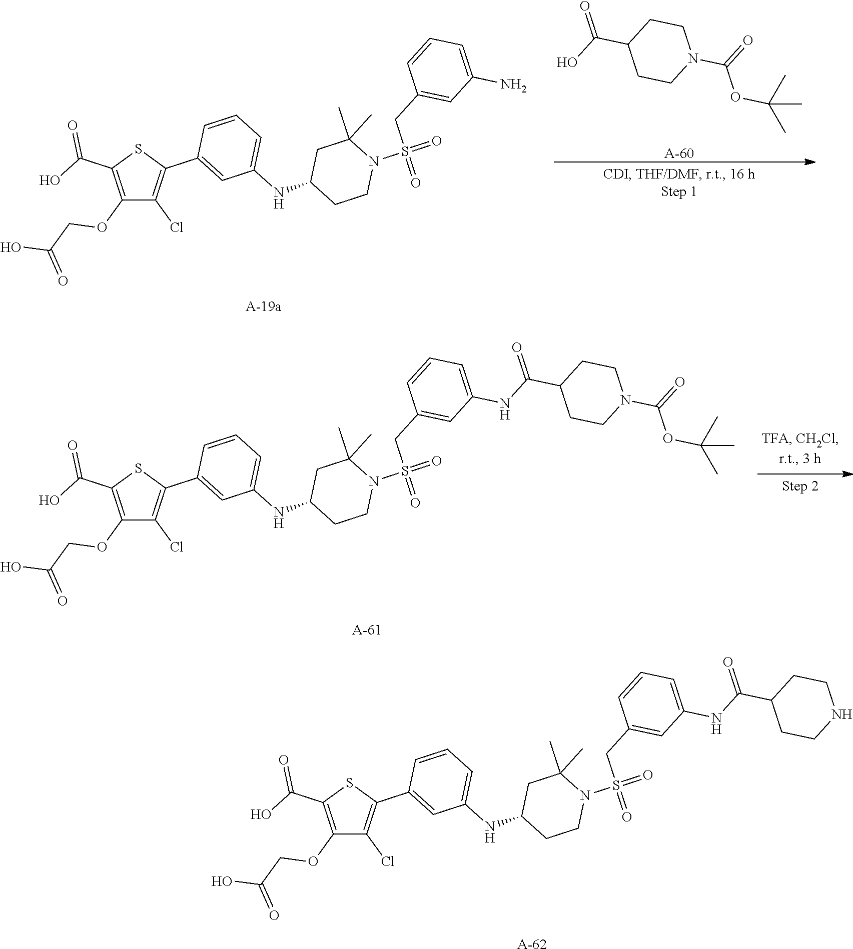

### Step 1: 5-[3-[[(4S)-1-[[3-[(1-tert-butoxycarbonylpiperidine-4-carbonyl)amino]phenyl]methylsulfonyl]-2,2-dimethyl-4-piperidyl]amino]phenyl]-3-(carboxymethoxy)-4-chloro-thiophene-2-carboxylic acid (A-61)

Into a 25 mL single neck round bottom flask containing a well-stirred solution of 1-tert-butoxycarbonylpiperidine-4-carboxylic acid (A-60, 50 mg, 218.08 μmol) in anhydrous THF (3 mL) and DMF (0.5 mL) was added 1,1′-carbonyldiimidazole (70.72 mg, 436.16 μmol). The reaction mixture was stirred for 2 h. Then, 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 66.31 mg, 109.04 μmol) was added. After 16 h, the solvent was removed under reduced pressure and the residue purified by reverse phase prep HPLC [Purification method: C18, Aq gold; Mobile phase A: 0.1% TFA in water; Mobile phase B: MeCN] to afford 5-[3-[[(4S)-1-[[3-[(1-tert-butoxycarbonylpiperidine-4-carbonyl)amino]phenyl]methylsulfonyl]-2,2-dimethyl-4-piperidyl]amino]phenyl]-3-(carboxymethoxy)-4-chloro-thiophene-2-carboxylic acid (A-61, 35 mg, 40.99 μmol, 19% yield) as an off-white solid. LCMS (ES+): m/z 819.3 [M+H]⁺.

### Step 2: 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62)

Into a 25 mL single neck round bottom flask containing a well-stirred solution of 5-[3-[[(4S)-1-[[3-[(1-tert-butoxycarbonylpiperidine-4-carbonyl)amino]phenyl]methylsulfonyl]-2,2-dimethyl-4-piperidyl]amino]phenyl]-3-(carboxymethoxy)-4-chloro-thiophene-2-carboxylic acid (A-61, 30 mg, 36.61 μmol) in anhydrous DCM (2 mL) was added TFA (740.00 mg, 6.49 mmol, 0.5 mL) at 0° C. The reaction mixture was stirred at room temperature for 2 h. The volatiles were removed under reduced pressure and azeotroped with toluene to afford 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62, 35 mg, 32.04 μmol, 92% yield, TFA salt) as an off-white solid, which was used in the next step without further purification. LCMS (ES+): m/z 719.2 [M+H]⁺.

### 5-[3-[[(4S)-1-[[3-(azetidine-3-carbonylamino)phenyl]methylsulfonyl]-2,2-dimethyl-4-piperidyl]amino]phenyl]-3-(carboxymethoxy)-4-chloro-thiophene-2-carboxylic acid (A-65)

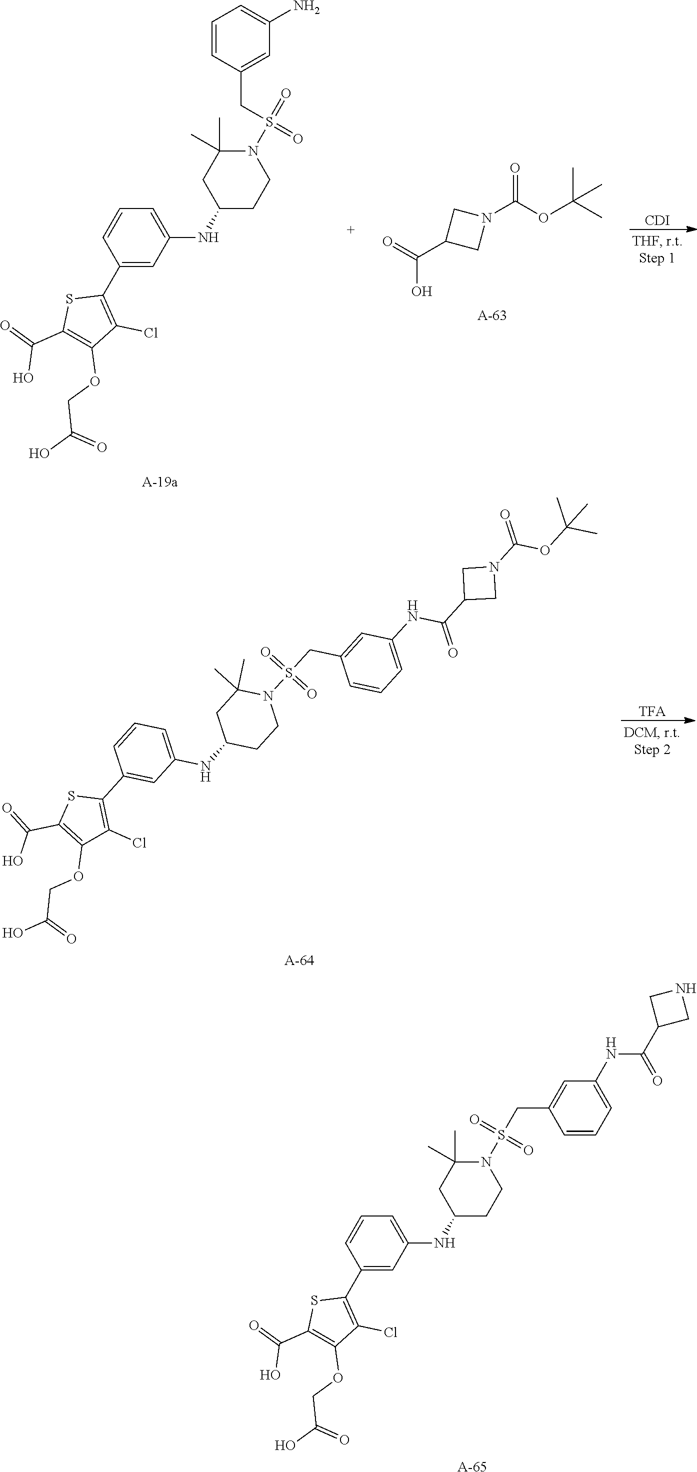

### Step 1: 5-[3-[[(4S)-1-[[3-[(1-tert-butoxycarbonylazetidine-3-carbonyl)amino]phenyl]methylsulfonyl]-2,2-dimethyl-4-piperidyl]amino]phenyl]-3-(carboxymethoxy)-4-chloro-thiophene-2-carboxylic acid (A-64)

5-[3-[[(4S)-1-[[3-[(1-tert-butoxycarbonylazetidine-3-carbonyl)amino]phenyl]methylsulfonyl]-2,2-dimethyl-4-piperidyl]amino]phenyl]-3-(carboxymethoxy)-4-chloro-thiophene-2-carboxylic acid (A-64, 55 mg, 56.50 μmol, 20% yield, TFA salt) was synthesized from 1-tert-butoxycarbonylazetidine-3-carboxylic acid (A-63) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Compound A-61, except using only THE. LCMS (ESI): m/z 791.3 [M+H]⁺.

### Step 2: 5-[3-[[(4S)-1-[[3-(azetidine-3-carbonylamino)phenyl]methylsulfonyl]-2,2-dimethyl-4-piperidyl]amino]phenyl]-3-(carboxymethoxy)-4-chloro-thiophene-2-carboxylic acid (A-65)

5-[3-[[(4S)-1-[[3-(azetidine-3-carbonylamino)phenyl]methylsulfonyl]-2,2-dimethyl-4-piperidyl]amino]phenyl]-3-(carboxymethoxy)-4-chloro-thiophene-2-carboxylic acid (A-65, 60 mg, 53.65 μmol, 88% yield, TFA salt) was synthesized from 5-[3-[[(4S)-1-[[3-[(1-tert-butoxycarbonylazetidine-3-carbonyl)amino]phenyl]methylsulfonyl]-2,2-dimethyl-4-piperidyl]amino]phenyl]-3-(carboxymethoxy)-4-chloro-thiophene-2-carboxylic acid (A-64) in a similar fashion to Compound A-62 except using 1 eq. TFA. LCMS (ESI): m/z 691.3 [M+H]⁺.

### 2-[[2-tert-butoxycarbonyl-4-chloro-5-[3-[[1-[[3-[3-(4-oxo-1-piperidyl)propanoylamino]phenyl]methylsulfonyl]-4-piperidyl]amino]phenyl]-3-thienyl]oxy]acetic acid (A-70)

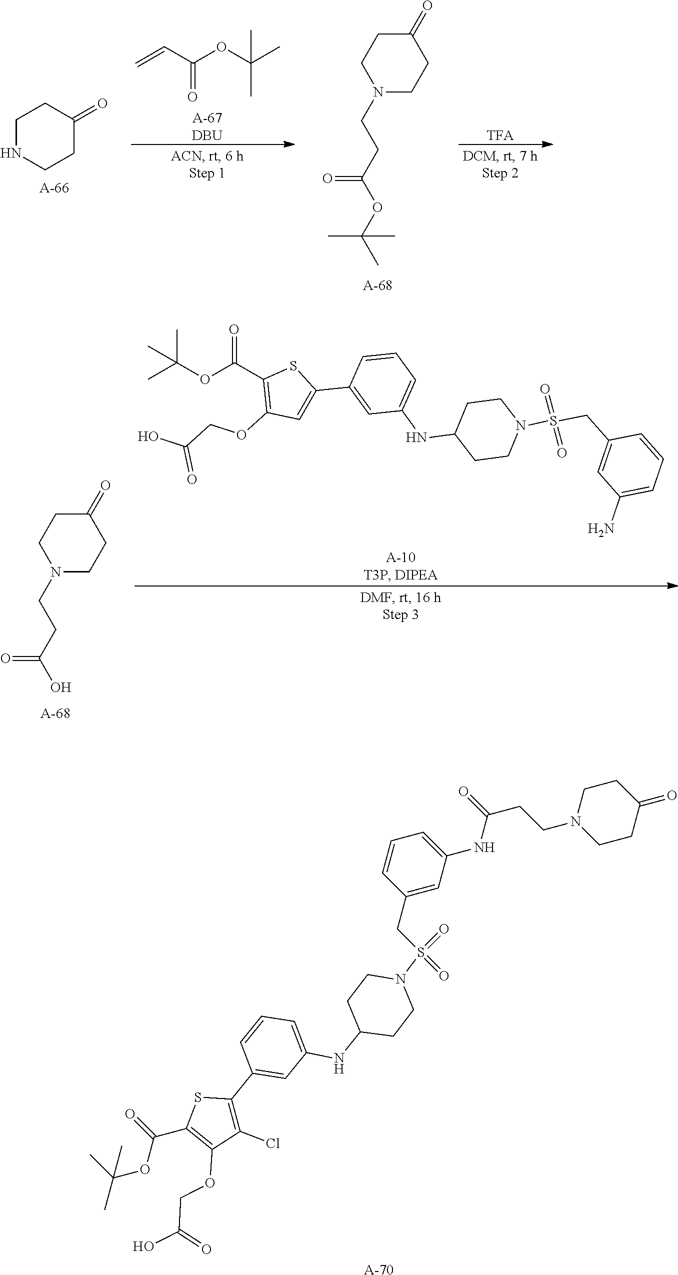

### Step 1: tert-butyl 3-(4-oxo-1-piperidyl)propanoate (A-68)

Into a 50 mL two neck round bottom flask containing a well-stirred suspension of piperidin-4-one hydrochloride (A-66, 500 mg, 3.69 mmol) in MeCN (10 mL) was added DBU (1.24 g, 8.11 mmol) dropwise over a period of 5 min at 0° C. After 10 min, tert-butyl prop-2-enoate (A-67, 567.15 mg, 4.43 mmol, 642.30 μL) was added dropwise over a period of 5 min. The resulting mixture was stirred at room temperature for 6 h. The reaction mixture was concentrated under reduced pressure and the residue was purified by flash silica gel column chromatography (2-5% of MeOH in DCM) to afford tert-butyl 3-(4-oxo-1-piperidyl)propanoate (A-68, 350 mg, 1.35 mmol, 37% yield) as a brown liquid. LCMS (ES+): m/z 228.1 [M+H]⁺.

### Step 2: 3-(4-oxopiperidin-1-yl)propanoic acid (A-69)

3-(4-oxo-1-piperidyl)propanoic acid (A-69, 340 mg, 1.09 mmol, 73% yield, TFA salt) was synthesized from tert-butyl 3-(4-oxo-1-piperidyl)propanoate (A-68) in a similar fashion to Compound A-26, except using 20 eq. TFA. Upon completion, the volatiles were removed under reduced pressure to afford the product as a light brown syrup, which was used in the next step without purification. LCMS (ES+): m/z 172.1 [M+H]⁺.

### Step 3: 2-[[2-tert-butoxycarbonyl-4-chloro-5-[3-[[1-[[3-[3-(4-oxo-1-piperidyl)propanoylamino]phenyl]methylsulfonyl]-4-piperidyl]amino]phenyl]-3-thienyl]oxy]acetic acid (A-70)

Into a 10 mL single neck round bottom flask containing a well-stirred solution of 3-(4-oxo-1-piperidyl)propanoic acid (A-69, 200 mg, 1.17 mmol, TFA salt) in DMF (4 mL) was added DIPEA (754.95 mg, 5.84 mmol, 1.02 mL) followed by 1-propanephosphonic anhydride (50% in ethyl acetate) (748.14 mg, 2.34 mmol). After 30 min, 2-[5-[3-[[1-[(3-aminophenyl)methylsulfonyl]-4-piperidyl]amino]phenyl]-2-tert-butoxycarbonyl-4-chloro-3-thienyl]oxy]acetic acid (A-10, 445.94 mg, 700.96 μmol). After 16 h, the solvent was removed under reduced pressure and the residue was subjected to reverse phase preparative HPLC (Column: Zorbax-SB18, 21.2×150 mm; Mobile phase A: 0.1% TFA in water; Mobile phase B: MeCN) to afford 2-[[2-tert-butoxycarbonyl-4-chloro-5-[3-[[1-[[3-[3-(4-oxo-1-piperidyl)propanoylamino]phenyl]methylsulfonyl]-4-piperidyl]amino]phenyl]-3-thienyl]oxy]acetic acid (A-70, 80 mg, 73.16 μmol, 6% yield, TFA salt) as an off white solid. LCMS (ES−): m/z 787.1 [M−H]⁻. tert-Butyl 5-(3-aminophenyl)-3-(2-(tert-butoxy)-2-oxoethoxy)-4-chlorothiophene-2-carboxylate (A-73)

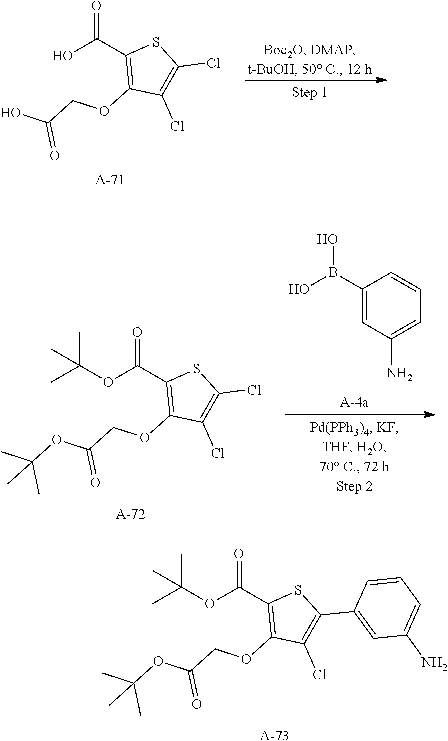

### Step 1: tert-Butyl 3-(2-(tert-butoxy)-2-oxoethoxy)-4,5-dichlorothiophene-2-carboxylate (A-72)

To a solution of 3-(carboxymethoxy)-4,5-dichlorothiophene-2-carboxylic acid (A-71, 1.0 g, 3.0 mmol) in t-BuOH (40 mL) at ambient temperature was added DMAP (0.036 g, 0.30 mmol), pyridine (0.96 mL, 12 mmol) and di-tert-butyl dicarbonate (3.4 mL, 15 mmol) as a solution in t-BuOH (4 mL). The mixture was heated to 50° C. and stirred for 12 h. The mixture was concentrated under reduced pressure and the residue purified by silica gel chromatography eluting with 10:1 petroleum ether:ethyl acetate to afford ter-butyl 3-(2-(tert-butoxy)-2-oxoethoxy)-4,5-dichlorothiophene-2-carboxylate (A-72, 698.2 mg, 1.59 mmol, 53% yield) as yellow oil. LCMS (ES+): m/z 270.6 [M−2/Bu+H]⁺.

### Step 2: tert-Butyl 5-(3-aminophenyl)-3-(2-(tert-butoxy)-2-oxoethoxy)-4-chlorothiophene-2-carboxylate (A-73)

tert-butyl 5-(3-aminophenyl)-3-(2-(tert-butoxy)-2-oxoethoxy)-4-chlorothiophene-2-carboxylate (A-73) was synthesized from tert-butyl 3-(2-(tert-butoxy)-2-oxoethoxy)-4,5-dichlorothiophene-2-carboxy late (A-72) and 3-aminophenylboronic acid monohydrate (A-4a) in a similar fashion to Compound A-5, except using THE as the solvent. Upon completion, the mixture was diluted with ethyl acetate and water. The organic layer was washed with brine, dried over sodium sulfate, filtered and concentrated under reduced pressure. The residue was purified by column chromatography on silica gel (petroleum ether:ethyl acetate=3:1) to afford tert-butyl 5-(3-aminophenyl)-3-(2-(tert-butoxy)-2-oxoethoxy)-4-chlorothiophene-2-carboxy late (A-73, 0.80 g, 1.5 mmol, 77% yield) as light yellow solid. LCMS (ES+): m/z 327.8 [M−2/Bu]⁺.

### tert-butyl (R)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74a, First Eluted Fraction) and tert-butyl(S)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74b, Second Eluted Fraction)

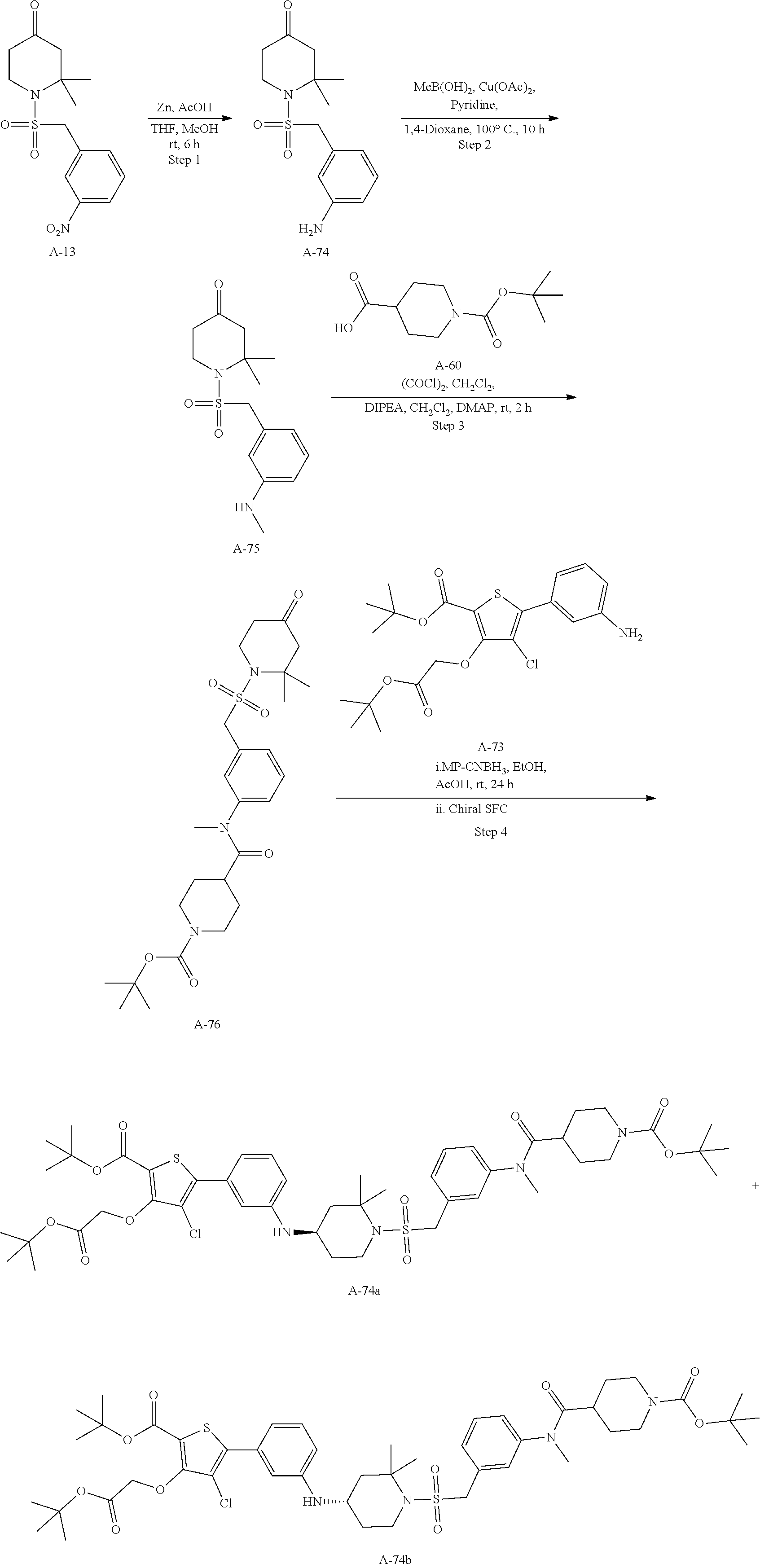

### Step 1: 1-[(3-aminophenyl)methylsulfonyl]-2,2-dimethyl-piperidin-4-one (A-74)

1-[(3-aminophenyl)methylsulfonyl]-2,2-dimethyl-piperidin-4-one (A-74, 1.8 g, 4.09 mmol, 67% yield)) was synthesized from 2,2-dimethyl-1-[(3-nitrophenyl)methylsulfonyl]piperidin-4-one (A-13) in a similar fashion to Compound A-10 except using 1 eq. Zinc and 1 eq. Acetic acid. Upon completion, the reaction was filtered through Celite washing with THE, DCM and Methanol. The solvent was removed under reduced pressure and residue diluted with ice cold water and stirred for 10 min. Precipitate was filtered and dried under vacuum and the solid triturated with diethyl ether. The material was used in the next step without further purification. LCMS (ES+): m/z 297.1 [M+H]⁺.

### Step 2: 2,2-dimethyl-1-((3-(methylamino)benzyl)sulfonyl)piperidin-4-one (A-75)

Into a 100 mL sealed tube containing a well-stirred solution of 1-[(3-aminophenyl)methylsulfonyl]-2,2-dimethyl-piperidin-4-one (A-74, 1.6 g, 5.40 mmol) in anhydrous 1,4-dioxane (20 mL) was added copper(II) acetate (1.18 g, 6.48 mmol) and pyridine (1.28 g, 16.20 mmol, 1.31 mL) at room temperature under nitrogen atmosphere. The resulting mixture was stirred at 100° C. for 30 min and cooled to room temperature. Methyl boronic acid (484.72 mg, 8.10 mmol) was added and the resulting mixture stirred at 100° C. for 16 h. The reaction mixture was poured over ice cold water and extracted with Ethyl acetate (2×50 mL). The combined organic layers were washed with brine (2×30 mL), dried over anhydrous Sodium sulfate and filtered. The filtrate was concentrated under reduced pressure and the residue purified by flash silica gel (100-200 mesh) column chromatography (80% Ethyl acetate in petroleum ether) to afford 2,2-dimethyl-1-((3-(methylamino)benzyl)sulfonyl)piperidin-4-one (A-75, 600 mg, 1.74 mmol, 32% yield) as an off-white solid. ¹H NMR (400 MHz, DMSO-d6): δ 7.07 (t, J=7.96 Hz, 1H), 6.59-6.50 (m, 3H), 5.72-5.71 (m, 1H), 4.29 (s, 2H), 3.61 (t, J=6.04 Hz, 2H), 2.67 (d, J=5.08 Hz, 3H), 2.54 (s, 2H), 2.26 (t, J=5.96 Hz, 3H), 1.38 (s, 3H), 1.37 (s, 3H).

### Step 3: tert-butyl4-((3-(((2,2-dimethyl-4-oxopiperidin-1-yl)sulfonyl)methyl)phenyl)(methyl)carbamoyl)piperidine-1-carboxylate (A-76)

Into a 50 mL single neck round bottom flask containing a well-stirred solution of 1-tert-butoxycarbonylpiperidine-4-carboxylic acid (A-60, 553.96 mg, 2.42 mmol) in anhydrous DCM (15 mL) was added oxalyl chloride (306.67 mg, 2.42 mmol, 210.05 μL) at 0° C. The reaction mixture was stirred at room temperature for 1 h. The volatiles were removed to obtain the crude acid chloride, which was taken up in DCM (15 mL) and treated with 2,2-dimethyl-1-((3-(methylamino)benzyl)sulfonyl)piperidin-4-one (A-75, 500 mg, 1.61 mmol), DIPEA (1.04 g, 8.05 mmol, 1.40 mL) and DMAP (19.68 mg, 161.08 μmol) at 0° C. After 1 h, the reaction mixture was diluted with DCM (60 mL) and washed with 1.5 N HCl (2×20 mL). The organic layer was dried over anhydrous Sodium sulfate, filtered and concentrated under reduced pressure. The residue was purified by flash silica gel (230-400 mesh) column chromatography (3% MeOH in DCM) to afford tert-butyl4-((3-(((2,2-dimethyl-4-oxopiperidin-1-yl)sulfonyl)methyl)phenyl)(methyl)carbamoyl)piperidine-1-carboxylate (A-76, 450 mg, 837.17 μmol, 52% yield) as light yellow solid. LCMS (ES+): m/z 422.2 [M−Boc+H]⁺.

### Step 4: tert-butyl (R)-4-(3-(4-(3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74a, First Eluted Fraction) and tert-butyl(S)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74b, Second Eluted Fraction)

Configurations are arbitrarily assigned.

Into a 50 mL single neck round bottom flask containing well-stirred solution of tert-butyl4-((3-(((2,2-dimethyl-4-oxopiperidin-1-yl)sulfonyl)methyl)phenyl)(methyl)carbamoyl)piperidine-1-carboxylate (A-76, 450 mg, 862.62 mol) and tert-butyl 5-(3-aminophenyl)-3-(2-tert-butoxy-2-oxo-ethoxy)-4-chloro-thiophene-2-carboxylate (A-73, 379.51 mg, 862.62 μmol) in anhydrous ethanol (20 mL) were added acetic acid (155.41 mg, 2.59 mmol, 148.01 μL) and MP-cyanoborohydride (800 mg, 1.6 mmol). After 24 h, the reaction mixture was filtered, concentrated under reduced pressure and the residue purified by flash silica gel (230-400 mesh) column chromatography (35% Ethyl acetate in petroleum ether) to obtain tert-butyl 4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74a/b), which was subjected to chiral SFC [Purification method: Column Name: Chiral CCS; Flow rate: 3 mL/min; Co-Solvent: 15%; Co-Solvent Name: 0.5% Isopropyl amine in methanol; Outlet Pressure: 100 bar; Injected Volume: 15 μl; Temperature: 35° C.] to afford tert-butyl (R)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxy late (A-74a, first eluted fraction, 140 mg, 142.43 μmol, 17% yield) as an off-white solid. LCMS (ES+): m/z 945.3 [M+H]⁺.

and tert-butyl(S)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74b, second eluted fraction, 120 mg, 109.77 μmol, 13% yield) as an off-white solid. LCMS (ES+): m/z 945.3 [M+H]⁺.

### 3-(carboxymethoxy)-4-chloro-5-[3-[[2,2-dimethyl-1-[[3-[methyl-[3-(4-oxo-1-piperidyl)propanoyl]amino]phenyl]methylsulfonyl]-4-piperidyl]amino]phenyl]thiophene-2-carboxylic acid (A-80)

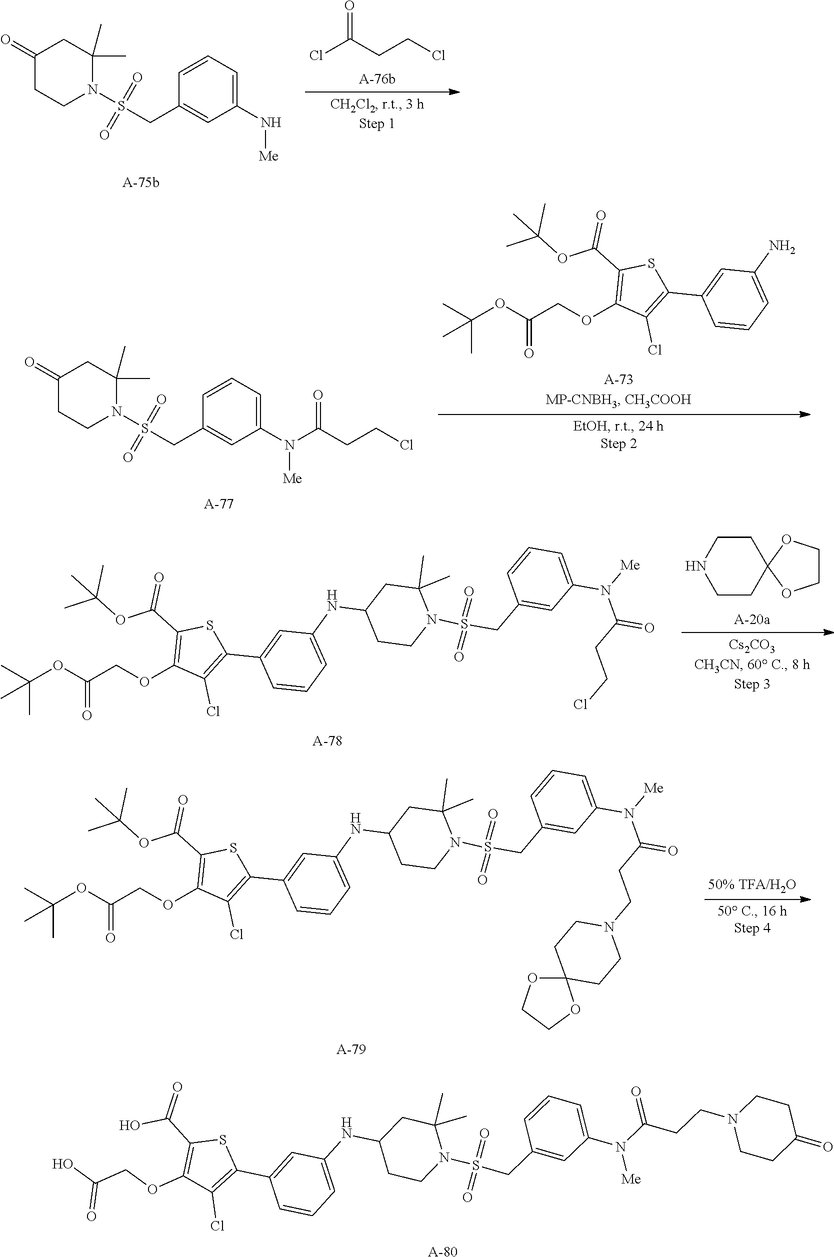

### Step 1: 3-chloro-N-[3-[(2,2-dimethyl-4-oxo-1-piperidyl)sulfonylmethyl]phenyl]-N-methyl-propanamide (A-77)

Into a 50 mL single neck round bottom flask containing well-stirred solution of 2,2-dimethyl-1-[[3-(methylamino)phenyl]methylsulfonyl]piperidin-4-one (A-75b, 300 mg, 966.46 μmol) in anhydrous DCM (15 mL) was added 3-chloropropanoyl chloride (A-76b, 184.07 mg, 1.45 mmol, 138.40 μL). After 3 h, the solvent was removed to afford 3-chloro-N-[3-[(2,2-dimethyl-4-oxo-1-piperidyl)sulfonylmethyl]phenyl]-N-methyl-propanamide (A-77, 310 mg, 607.44 μmol, 63% yield) as a sticky brown solid. This material was used in the next step without purification. LCMS (ES+): m/z 401.2 [M+H]⁺.

### Step 2: tert-butyl 3-(2-tert-butoxy-2-oxo-ethoxy)-4-chloro-5-[3-[[1-[[3-[3-chloropropanoyl(methyl)amino]phenyl]methylsulfonyl]-2,2-dimethyl-4-piperidyl]amino]phenyl]thiophene-2-carboxylate (A-78)

tert-butyl 3-(2-tert-butoxy-2-oxo-ethoxy)-4-chloro-5-[3-[[1-[[3-[3-chloropropanoyl(methyl)amino]phenyl]methylsulfonyl]-2,2-dimethyl-4-piperidyl]amino]phenyl]thiophene-2-carboxy late (A-78, 320 mg, 318.11 μmol, 41% yield) was synthesized from tert-butyl 5-(3-aminophenyl)-3-(2-tert-butoxy-2-oxo-ethoxy)-4-chloro-thiophene-2-carboxylate (A-73) and 3-chloro-N-[3-[(2,2-dimethyl-4-oxo-1-piperidyl)sulfonylmethyl]phenyl]-N-methyl-propanamide (A-77) in a similar fashion to Compound A-74a/b. LCMS (ES+): m/z 822.0 [M−H]⁻.

### Step 3: tert-butyl 3-(2-tert-butoxy-2-oxo-ethoxy)-4-chloro-5-[3-[[1-[[3-[3-(1,4-dioxa-8-azaspiro[4.5]decan-8-yl)propanoyl-methyl-amino]phenyl]methylsulfonyl]-2,2-dimethyl-4-piperidyl]amino]phenyl]thiophene-2-carboxylate (A-79)

Into a 50 mL sealed tube containing a well-stirred solution of ter-butyl 3-(2-tert-butoxy-2-oxo-ethoxy)-4-chloro-5-[3-[ ]-[[3-[3-chloropropanoyl(methyl)amino]phenyl]methylsulfonyl]-2,2-dimethyl-4-piperidyl]amino]phenyl]thiophene-2-carboxylate (A-78, 0.6 g, 727.39 μmol), 1,4-dioxa-8-azaspiro[4.5]decane (A-20a, 208.30 mg, 1.45 mmol, 185.98 μL) in anhydrous acetonitrile (15 mL) was added Cs₂CO₃ (473.99 mg, 1.45 mmol) at room temperature. The mixture was stirred at 60° C. for 8 h. The reaction mixture was filtered, concentrated under reduced pressure and the residue purified by reverse phase prep HPLC [Purification method: Column: SunFire C18 (19×150 mm) 5 μm, mobile phase: 0.1% TFA in water and MeCN] to afford tert-butyl 3-(2-tert-butoxy-2-oxo-ethoxy)-4-chloro-5-[3-[[1-[[3-[3-(1,4-dioxa-8-azaspiro[4.5]decan-8-yl)propanoyl-methyl-amino]phenyl]methylsulfonyl]-2,2-dimethyl-4-piperidyl]amino]phenyl]thiophene-2-carboxylate (A-79, 350 mg, 334.03 μmol, 46% yield, TFA salt) as off-white solid. LCMS (ES+): m/z 931.4 [M+H]⁺.

### Step 4: 3-(carboxymethoxy)-4-chloro-5-[3-[[2,2-dimethyl-1-[[3-[methyl-[3-(4-oxo-1-piperidyl)propanoyl]amino]phenyl]methylsulfonyl]-4-piperidyl]amino]phenyl]thiophene-2-carboxylic acid (A-80)

Into a 50 mL single neck round bottom flask containing well-stirred solution of tert-butyl 3-(2-tert-butoxy-2-oxo-ethoxy)-4-chloro-5-[3-[[1-[[3-[3-(1,4-dioxa-8-azaspiro[4.5]decan-8-yl)propanoyl-methyl-amino]phenyl]methylsulfonyl]-2,2-dimethyl-4-piperidyl]amino]phenyl]thiophene-2-carboxy late (A-79, 300 mg, 322.03 μmol) in water (3.00 mL) was added TFA (4.44 g, 38.94 mmol, 3.00 mL) at room temperature. The contents were stirred at 50° C. for 16 h. The solvent was removed to afford 3-(carboxymethoxy)-4-chloro-5-[3-[2,2-dimethyl-1-[[3-[methyl-[3-(4-oxo-1-piperidyl)propanoyl]amino]phenyl]methylsulfonyl]-4-piperidyl]amino]phenyl]thiophene-2-carboxylic acid (A-80, 300 mg, 182.16 μmol, 57% yield, TFA salt) as light brown sticky solid. The material was used in the next step without purification. LCMS (ES+): m/z 775.4 [M+H]⁺.

### 5-[3-[[(4S)-1-[[3-(azetidin-3-yloxycarbonylamino)phenyl]methylsulfonyl]-2,2-dimethyl-4-piperidyl]amino]phenyl]-3-(carboxymethoxy)-4-chloro-thiophene-2-carboxylic acid (A-86)

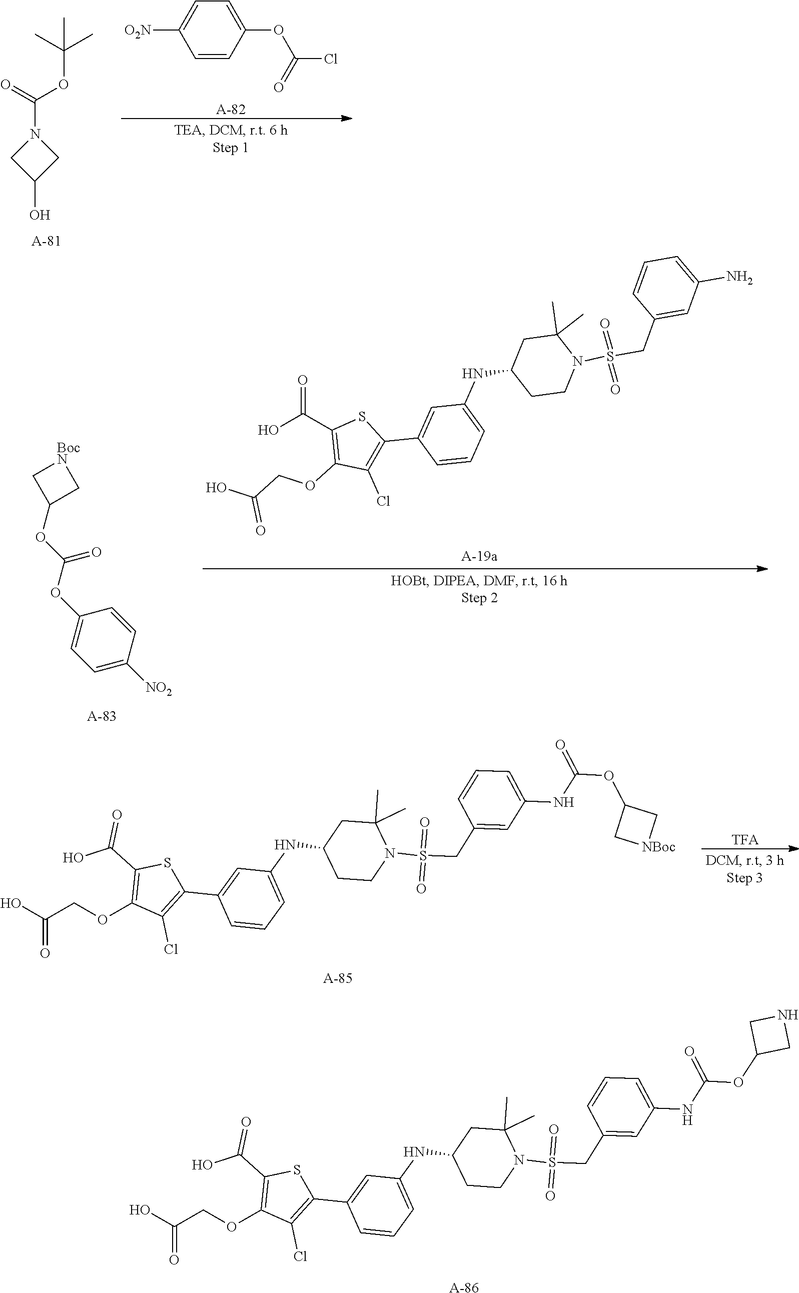

### Step 1: tert-butyl 3-(4-nitrophenoxy)carbonyloxyazetidine-1-carboxylate (A-83)

Into a 50 mL single neck round bottom flask containing a well-stirred solution of tert-butyl 3-hydroxyazetidine-1-carboxylate (A-81, 1 g, 5.77 mmol) in anhydrous DCM (10 mL) were added triethylamine (1.75 g, 17.32 mmol, 2.41 mL) and 4-nitrophenyl chloroformate (A-82, 1.75 g, 8.66 mmol) at 0° C. under nitrogen atmosphere. The resulting solution was stirred at room temperature for 6 h. The reaction mixture was diluted with water (30 mL) and extracted with DCM (2×20 mL). The combined organic layer was washed with brine solution (20 mL), dried over anhydrous sodium sulfate, filtered and concentrated under reduced pressure to afford tert-butyl 3-(4-nitrophenoxy)carbonyloxyazetidine-1-carboxylate (A-83, 500 mg, 1.00 mmol, 17% yield) as a pale yellow colour viscus oil. The material was used in the next step without purification. LCMS (ES+): m/z 239.1 [M−Boc+H]⁺.

### Step 2: 5-[3-[[(4S)-1-[[3-[(1-tert-butoxycarbonylazetidin-3-yl)oxycarbonylamino]phenyl]methylsulfonyl]-2,2-dimethyl-4-piperidyl]amino]phenyl]-3-(carboxymethoxy)-4-chloro-thiophene-2-carboxylic acid (A-85)

Into a 10 mL single neck round bottom flask containing a well-stirred solution of 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 100 mg, 0.164 mmol) in anhydrous DMF (1 mL) were added tert-butyl 3-(4-nitrophenoxy)carbonyloxyazetidine-1-carboxylate (A-83, 69.54 mg, 0.205 mmol), DIPEA (42.50 mg, 0.328 mmol, 57.28 μL) and HOBt (23.33 mg, 0.172 mmol). After 16 h, the solvent was removed under reduced pressure and the residue was washed with diethyl ether (2×15 mL) and concentrated under reduced pressure. The residue was purified by reverse phase column chromatography [Purification method: Column: XSelect (150×19) mm, 5 um; Mobile phase A: 0.1% TFA in MQ-water; Mobile phase B: Acetonitrile] to afford 5-[3-[[(4S)-1-[[3-[(1-tert-butoxycarbonylazetidin-3-yl)oxycarbonylamino]phenyl]methylsulfonyl]-2,2-dimethyl-4-piperidyl]amino]phenyl]-3-(carboxymethoxy)-4-chloro-thiophene-2-carboxylic acid (A-85, 15 mg, 0.015 mmol, 9% yield, TFA salt) as an off-white solid. LCMS (ES−): m/z 805.1 [M−H]⁻.

### Step 3: 5-[3-[[(4S)-1-[3-(azetidin-3-yloxycarbonylamino)phenyl]methylsulfonyl]-2,2-dimethyl-4-piperidyl]amino]phenyl]-3-(carboxymethoxy)-4-chloro-thiophene-2-carboxylic acid (A-86)

5-[3-[[(4S)-1-[[3-(azetidin-3-yloxycarbonylamino)phenyl]methylsulfonyl]-2,2-dimethyl-4-piperidyl]amino]phenyl]-3-(carboxymethoxy)-4-chloro-thiophene-2-carboxylic acid (A-86, 15 mg, 0.016 mmol, 88% yield, TFA salt) was synthesized from 5-[3-[[(4S)-1-[[3-[(1-tert-butoxycarbonylazetidin-3-yl)oxycarbonylamino]phenylmethylsulfonyl]-2,2-dimethyl-4-piperidyl]amino]phenyl]-3-(carboxymethoxy)-4-chloro-thiophene-2-carboxylic acid (A-85) in a similar fashion to Compound A-62, except using 10 eq. TFA. LCMS (ES+): m/z 707.1 [M+H]⁺.

### 5-[3-[[(4S)-1-[3-[(1S,5R)-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-89)

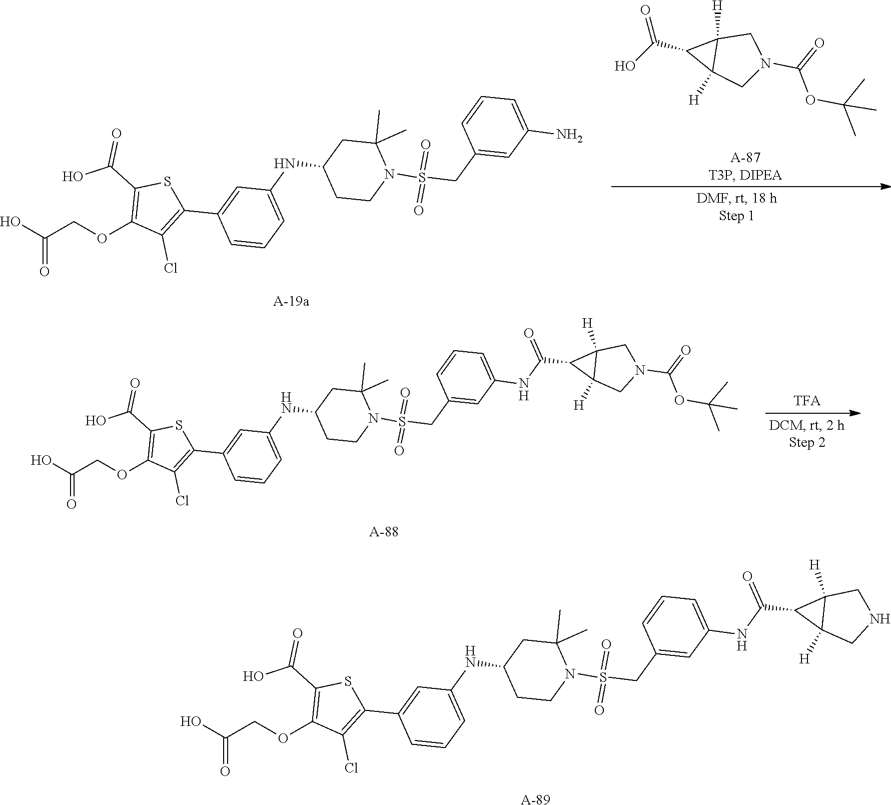

### Step 1: 5-[3-[[(4S)-1-[[3-[[(1S,5R)-3-tert-butoxycarbonyl-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-88)

Into a capped vial containing a well-stirred solution of (1S,5R)-3-tert-butoxycarbonyl-3-azabicyclo[3.1.0]hexane-6-carboxylic acid (A-87, 150.00 mg, 660.05 μmol) in DMF (1 mL) was added DIPEA (426.53 mg, 3.30 mmol, 574.84 μL) followed by 1-propanephosphonic anhydride (50% in ethyl acetate) (273.02 mg, 858.06 μmol, 0.55 mL). After 5 min, 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 321.11 mg, 528.04 μmol) was added. After 16 h, the volatiles were removed under reduced pressure, and the residue purified by reverse phase column chromatography (50-55% of MeCN in 0.1% TFA in water) to afford 5-[3-[[(4S)-1-[3-[(1S,5R)-3-tert-butoxycarbonyl-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-88, 95 mg, 100.91 μmol, 15% yield, TFA salt) as a pale yellow solid. LCMS (ES+): m/z 817.1 [M+H]⁺.

### Step 2: 5-[3-[[(4S)-1-[3-[(1S,5R)-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-89)

5-[3-[[(4S)-1-[3-[(1S,5R)-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-89, 80 mg, 93.36 μmol, 97% yield, TFA salt) was synthesized from 5-[3-[[(4S)-1-[3-[(1S,5R)-3-tert-butoxycarbonyl-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-88) in a similar fashion to Compound A-62, except using 68 eq. TFA and triturating the product with diethyl ether. LCMS (ES+): m/z 717.1 [M+H]⁺.

### 5-[3-[[(4S)-1-[[3-[[(3aR,6aS)-1,2,3,3a,4,5,6,6a-octahydrocyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-92)

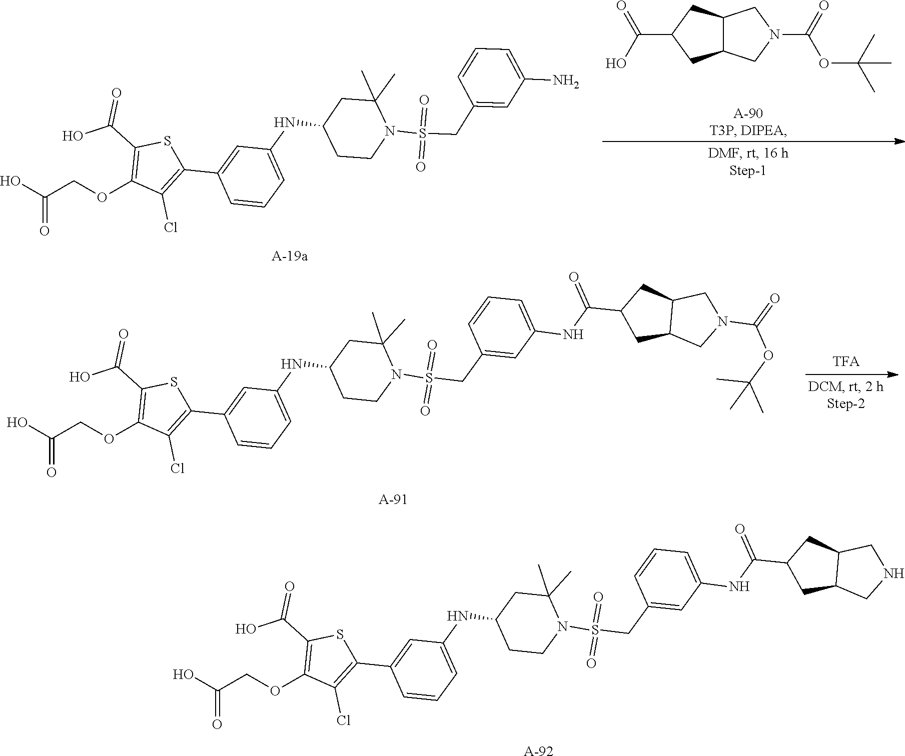

### Step 1: 5-[3-[[(4S)-1-[[3-[[(3aR,6aS)-2-tert-butoxycarbonyl-3,3a,4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-91)

5-[3-[[(4S)-1-[[3-[[(3aR,6aS)-2-tert-butoxycarbonyl-3,3a,4,5,6,6a-hexahydro-1H-cyclopenta[c]pyrrole-5-carbonyl]amino]phenylmethylsulfonyl]-2,2-dimethyl-4-piperidyl]amino]phenyl]-3-(carboxymethoxy)-4-chloro-thiophene-2-carboxylic acid (A-91, 87 mg, 93.19 μmol, 16% yield, formate salt) was synthesized from (3aR,6aS)-2-tert-butoxycarbonyl-3,3a,4,5,6,6a-hexahydro-1H-cyclopenta[c]pyrrole-5-carboxylic acid (A-90) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Compound A-88. LCMS (ES+): m/z 846.1 [M+H]⁺.

### Step 2: 5-[3-[[(4S)-1-[[3-[[(3aR,6aS)-1,2,3,3a,4,5,6,6a-octahydrocyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-92)

5-[3-[[(4S)-1-[[3-[(3aR,6aS)-1,2,3,3a,4,5,6,6a-octahydrocyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-92, 87 mg, 91.11 μmol, 90% yield, TFA salt) was synthesized from 5-[3-[[(4S)-1-[[3-[[(3aR,6aS)-2-tert-butoxycarbonyl-3,3a,4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-91b) in a similar fashion to Compound A-62, except using 74 eq. TFA and triturating the product with diethyl ether. LCMS (ES+): m/z 745.2 [M+H]⁺.

### tert-butyl 3-[[5-[(4R)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97a, First Eluted Fraction) and tert-butyl 3-[[5-[[(4S)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97b, Second Eluted Fraction)

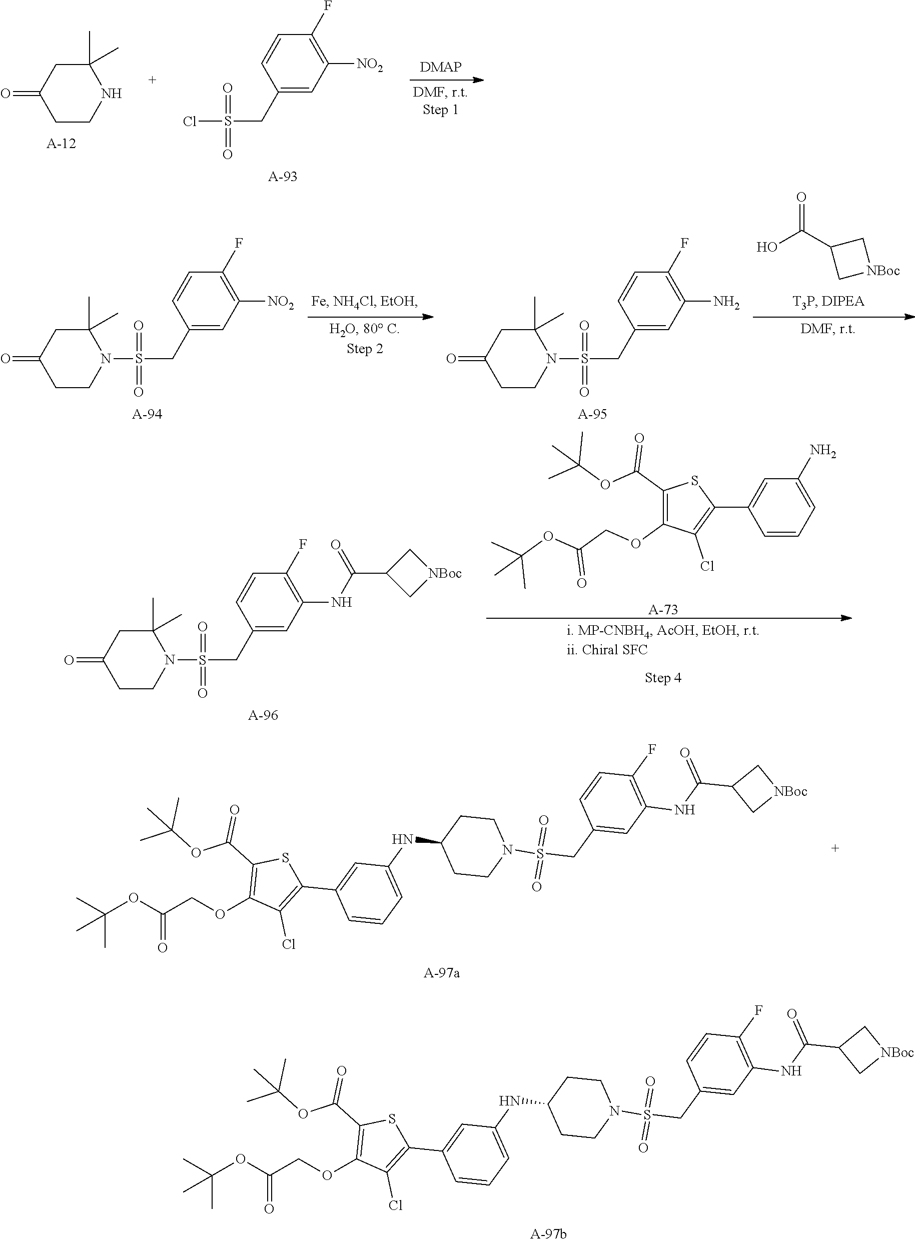

### Step 1: 1-[(4-fluoro-3-nitro-phenyl)methylsulfonyl]-2,2-dimethyl-piperidin-4-one (A-94)

Into a 50 mL single neck round bottom flask containing a well-stirred solution of 2,2-dimethylpiperidin-4-one hydrochloride (A-12, 1.0 g, 6.11 mmol) and (4-fluoro-3-nitro-phenyl)methanesulfonyl chloride (A-93, 6.20 g, 24.44 mmol) in anhydrous DMF (15 mL) was added DMAP (1.49 g, 12.22 mmol). After 16 h, the reaction mixture was diluted with water (20 mL) and extracted with EtOAc (2×20 mL). The combined organic layers were washed with brine (10 mL), dried over sodium sulfate, filtered and concentrated under reduced pressure. The residue was purified by flash silica-gel (230-400 mesh) column with 50-60% EtOAc/pet ether to afford 1-[(4-fluoro-3-nitro-phenyl)methylsulfonyl]-2,2-dimethyl-piperidin-4-one (A-94, 300 mg, 0.59 mmol, 10% yield) as a pale-yellow solid. LCMS (ESI): m/z 343.1 [M−H]⁻. ¹H NMR (400 MHz, DMSO-d₆). δ 8.26-8.24 (m, 1H), 7.89-7.85 (m, 1H), 7.67-7.62 (m, 1H), 4.63 (s, 2H), 3.68 (t, J=5.6 Hz, 2H), 2.59 (s, 2H), 2.40 (t, J=6 Hz, 2H) and 1.38 (s, 6H).

### Step 2:1-[(3-amino-4-fluoro-phenyl)methylsulfonyl]-2,2-dimethyl-piperidin-4-one (A-95)

Into a 50 mL single neck round bottom flask containing a well-stirred solution of 1-[(4-fluoro-3-nitro-phenyl)methylsulfonyl]-2,2-dimethyl-piperidin-4-one (A-94, 300 mg, 0.87 mmol) in a mixture of water (8 mL) and EtOH (8 mL) were added Fe powder (243.26 mg, 4.36 mmol) and ammonium chloride (233.01 mg, 4.36 mmol). The resulting mixture was stirred at 85° C. for 4 h. The reaction mixture was filtered through a pad of Celite and washed with dichloromethane (10 mL). Filtrate was diluted with water (10 mL) and extracted with DCM (2×10 mL). The combined organic layers were dried over Na₂SO₄, filtered and concentrated under reduced pressure to afford 1-[(3-amino-4-fluoro-phenyl)methylsulfonyl]-2,2-dimethyl-piperidin-4-one (A-95, 300 mg, 0.77 mmol, 89% yield) as a pale-yellow solid. This material was used in the next step without purification. LCMS (ESI): m/z 315.0 [M+H]⁺. ¹H NMR (400 MHz, DMSO-d₆), δ 6.99-6.94 (m, 1H), 6.83 (dd, J=8.8, 2.4 Hz, 1H), 6.55-6.51 (m, 1H), 5.23 (s, 2H), 4.27 (m, 2H), 3.60 (d, J=6.0 Hz, 2H), 2.56 (s, 2H), 2.30 (d, J=6.0 Hz, 2H) and 1.38 (s, 6H).

### Step 3: tert-butyl 3-[[5-[(2,2-dimethyl-4-oxo-1-piperidyl)sulfonylmethyl]-2-fluoro-phenyl]carbamoyl]azetidine-1-carboxylate (A-96)

tert-butyl 3-[[5-[(2,2-dimethyl-4-oxo-1-piperidyl)sulfonylmethyl]-2-fluoro-phenyl]carbamoyl]azetidine-1-carboxylate (A-96, 300 mg, 0.39 mmol, 41% yield) was synthesized from 1-[(3-amino-4-fluoro-phenyl)methylsulfonyl]-2,2-dimethyl-piperidin-4-one (A-95) and 1-tert-butoxycarbonylazetidine-3-carboxylic acid (A-63) in a similar fashion to Compound A-88 except using 2 eq. 1-propanephosphonic anhydride (50% in ethyl acetate) and 1.5 eq. A-63. Upon completion, the reaction mixture was diluted water and extracted with Ethyl acetate. Combined organic layers were dried over Sodium sulfate, filtered and concentrated under reduced pressure. This material was used in the next step without further purification. LCMS (ESI): m/z 496.1 [M−H]⁻. ¹H NMR (400 MHz, DMSO-d₆): δ9.94 (s, 1H), 8.03-7.96 (s, 1H), 7.31-7.26 (m, 1H), 7.22-7.19 (m, 1H), 4.44 (s, 2H), 4.02-3.93 (m, 5H), 3.64-3.60 (m, 3H), 2.57 (s, 2H), 2.34 (t, J=6 Hz, 1H), 1.41-1.36 (m, 15H).

### Step 4: tert-butyl 3-[[5-[[(4R)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97a, First Eluted Fraction) and tert-butyl 3-[[5-[[(4S)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97b, Second Eluted Fraction)

tert-butyl 3-[5-[(4R)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97a) and tert-butyl 3-[[5-[[(4S)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97b) were synthesized from tert-butyl 3-[[5-[(2,2-dimethyl-4-oxo-1-piperidyl)sulfonylmethyl]-2-fluoro-phenyl]carbamoyl]azetidine-1-carboxylate (A-96) and tert-butyl 5-(3-aminophenyl)-3-(2-tert-butoxy-2-oxo-ethoxy)-4-chloro-thiophene-2-carboxylate (A-73) in a similar fashion to Compound A-74a/b except using 1 eq. MP-cyanoborohydride and 29 eq. Acetic acid. tert-butyl 3-((5-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)azetidine-1-carboxy late (A-97a/b) was subjected to SFC chiral HPLC separation following the method: Column: (R,R)-Whelk-01; Co-Solvent: 0.5% ⁱPrNH₂ in 1:1 MeOH:IPA, Injected volume: 15 μL; Flow rate: 4 mL/min; RT=5.15 min to afford tert-butyl 3-[[5-[[(4R)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97a, first eluted fraction, 100 mg, 0.11 mmol, 18% yield) as an off-white solid. LCMS (ESI+): m/z 921.0 [M+H]⁺. ¹H NMR (400 MHz, DMSO-d₆): δ 9.92 (s, 1H), 8.03-8.00 (m, 1H), 7.30-7.26 (m, 1H), 7.21-7.17 (m, 2H), 6.86 (s, 1H), 6.81 (d, J=7.6 Hz, 1H), 6.70-6.67 (m, 1H), 5.68 (d, J=7.6 Hz, 1H), 4.85 (s, 2H), 4.46-4.30 (m, 2H), 4.01-3.93 (m, 5H), 3.65-3.61 (m, 1H), 3.51-3.43 (m, 2H), 3.15-3.08 (m, 1H), 1.88-1.77 (m, 2H), 1.52 (s, 9H), 1.46-1.38 (m, 25H), and RT=5.94 min to afford tert-butyl 3-[5-[[(4S)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97b, second eluted fraction, 100 mg, 0.107 mmol, 18% yield) as an off-white solid. LCMS (ESI−): m/z 919.2 [M−H]⁻. ¹H NMR (400 MHz, DMSO-d₆): δ 9.92 (s, 1H), 8.03-8.00 (s, 1H), 7.30-7.26 (m, 1H), 7.21-7.17 (m, 2H), 6.86 (s, 1H), 6.86-6.80 (d, 1H), 6.70-6.67 (m, 1H), 5.68 (d, J=8 Hz, 1H), 4.85 (s, 2H), 4.58-4.42 (m, 1H), 4.33-4.30 (m, 1H), 4.01-3.92 (m, 4H), 3.65-3.61 (m, 2H), 3.53-3.51 (m, 2H), 3.45-3.42 (m, 1H), 3.12 (t, J=12.4 Hz, 1H), 1.88 (d, J=11.6 Hz, 1H), 1.79 (d, J=9.6 Hz, 1H), 1.52 (s, 9H), 1.46-1.38 (m, 24H).

### tert-butyl (1R,5S,6r)-6-((5-((((S)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99a, First Eluted Fraction) and tert-butyl (1R,5S,6r)-6-((5-((((R)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99b, Second Eluted Fraction)

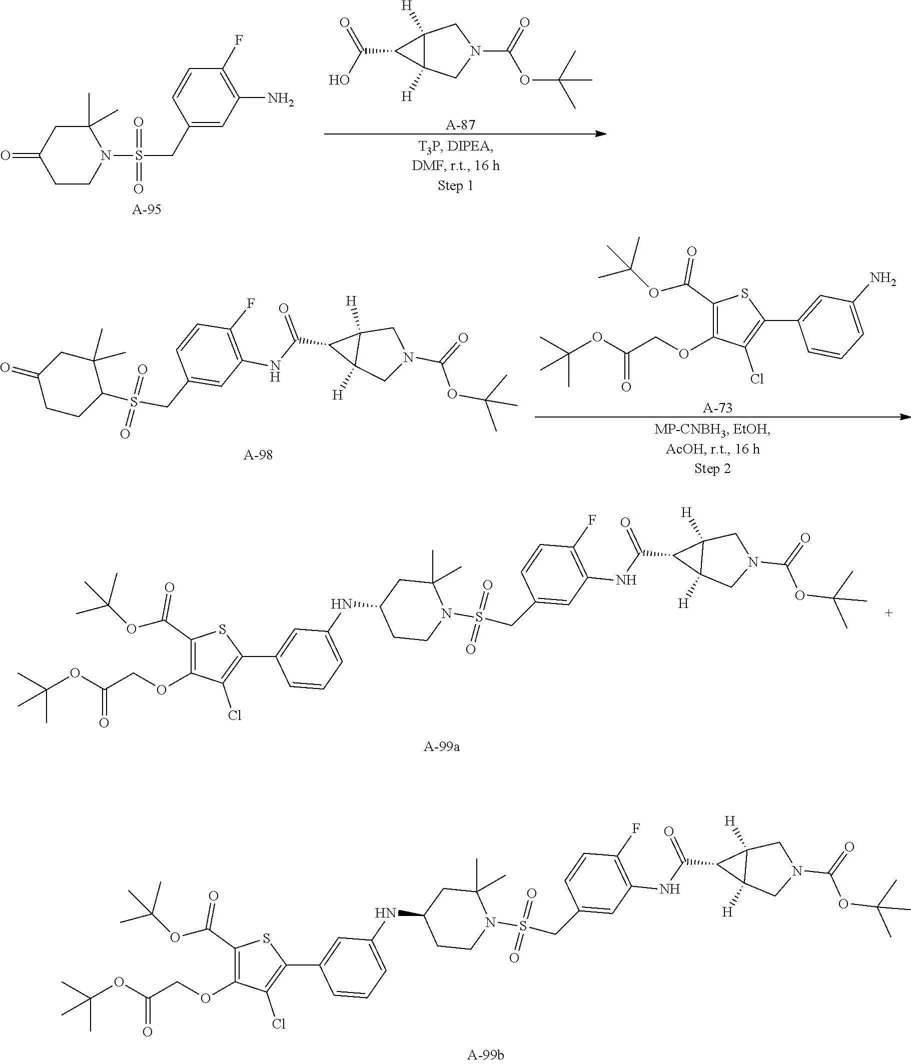

### Step 1: tert-butyl (1R,5S,6r)-6-((5-(((2,2-dimethyl-4-oxopiperidin-1-yl)sulfonyl)methyl)-2-fluorophenyl)carbamoyl)-3-azabicyclo[3.1.0]hexane-3-carboxylate (A-98)

tert-butyl (1S,5R)-6-[5-[(2,2-dimethyl-4-oxo-1-piperidyl)sulfonylmethyl]-2-fluoro-phenyl]carbamoyl]-3-azabicyclo[3.1.0]hexane-3-carboxylate (A-98, 320 mg, 505.41 μmol, 53% yield) was synthesized from 1-[(3-amino-4-fluoro-phenyl)methylsulfonyl]-2,2-dimethyl-piperidin-4-one (A-95) and (1S,5R)-3-tert-butoxycarbonyl-3-azabicyclo[3.1.0]hexane-6-carboxylic acid (A-87) in a similar fashion to Compound A-88 except using 1 eq. acid, 2 eq. 1-propanephosphonic anhydride (50% in ethyl acetate) and 4 eq. DIPEA. Upon completion, the reaction was quenched with cold water and extracted with ethyl acetate (3×). Combined organic layer was washed with 1.5 N aqueous HCl (2×15 mL), dried over anhydrous sodium sulfate, filtered and concentrated under reduced pressure. LCMS (ES−): m/z 522.1 [M−H]⁻.

### Step 2: tert-butyl (1R,5S,6r)-6-((5-((((S)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99a, First Eluted Fraction) and tert-butyl (1R,5S,6r)-6-((5-((((R)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99b, Second Eluted Fraction)

tert-butyl (1R,5S,6r)-6-((5-((((S)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99a, first eluted fraction) and tert-butyl (1R,5S,6r)-6-((5-((((R)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99b, second eluted fraction) were synthesized from tert-butyl (1S,5R)-6-[[5-[(2,2-dimethyl-4-oxo-1-piperidyl)sulfonylmethyl]-2-fluoro-phenyl]carbamoyl]-3-azabicyclo[3.1.0]hexane-3-carboxylate (A-98) and tert-butyl 5-(3-aminophenyl)-3-(2-tert-butoxy-2-oxo-ethoxy)-4-chloro-thiophene-2-carboxylate (A-73) in a similar fashion to Compound A-74a/b except using 28 eq. Acetic acid and 2.5 eq. MP-cyanoborohydride. tert-butyl (1R,5S,6r)-6-((5-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99a/b) was subjected to chiral SFC to separate isomers. [Purification method: Column Name: Lux A1; Flow rate: 3 mL/min; Co-Solvent: 30%; Co-Solvent Name: 0.5% Isopropyl Amine in IPA; Outlet Pressure: 100 bar; Injected Volume: 9 μl; Temperature: 35° C.] to afford tert-butyl (1R,5S,6r)-6-((5-((((S)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99a, first eluted fraction, 90 mg, 91.09 μmol, 15% yield) as an off-white solid. LCMS (ES−): m/z 945.2 [M−H]⁻.

and tert-butyl (1R,5S,6r)-6-((5-((((R)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99b, second eluted fraction, 70 mg, 70.42 μmol, 12% yield) as an off-white solid. LCMS (ES−): m/z 945.2 [M−H]⁻.

### tert-butyl 5-[3-[[(4S)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101a, First Eluted Fraction) and tert-butyl 5-[3-[[(4R)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101b, Second Eluted Fraction)

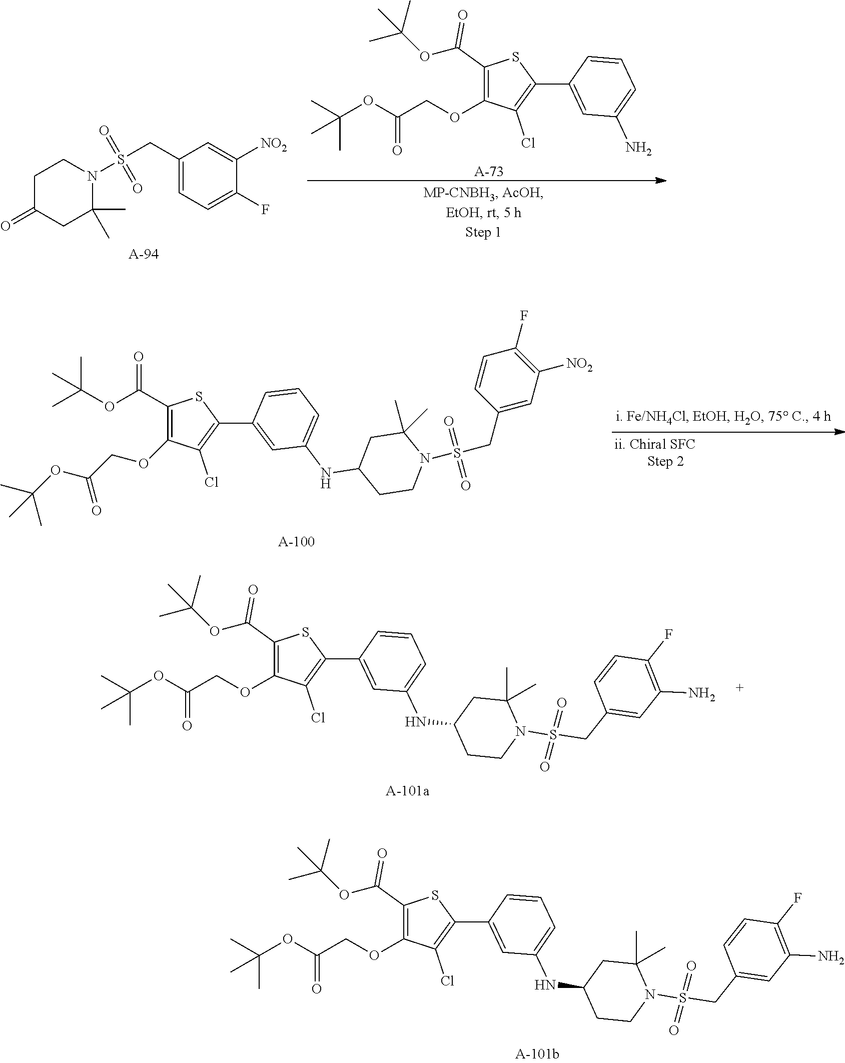

### Step 1; tert-butyl 3-(2-tert-butoxy-2-oxo-ethoxy)-4-chloro-5-[3-[[1-[(4-fluoro-3-nitro-phenyl)methylsulfonyl]-2,2-dimethyl-4-piperidyl]amino]phenyl]thiophene-2-carboxylate (A-100)

tert-butyl 3-(2-tert-butoxy-2-oxo-ethoxy)-4-chloro-5-[3-[[1-[(4-fluoro-3-nitro-phenyl)methylsulfonyl]-2,2-dimethyl-4-piperidyl]amino]phenyl]thiophene-2-carboxylate (A-100, 300 mg, 324.09 μmol, 56% yield) was synthesized from 1-[(4-fluoro-3-nitro-phenyl)methylsulfonyl]-2,2-dimethyl-piperidin-4-one (A-94) and 5-(3-aminophenyl)-3-(2-tert-butoxy-2-oxo-ethoxy)-4-chloro-thiophene-2-carboxylate (A-73) in a similar fashion to Compound A-74a/b except using 1 eq. MP-cyanoborohydride and 30 eq. Acetic acid. LCMS (ES+): m/z 657.3 [M−2 tBu+H]⁺.

### Step 2: tert-butyl 5-[3-[[(4S)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101a, First Eluted Fraction) and tert-butyl 5-[3-[[(4R)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101b, Second Eluted Fraction)

Into a 10 mL single neck round bottom flask containing a well-stirred solution of tert-butyl 3-(2-tert-butoxy-2-oxo-ethoxy)-4-chloro-5-[3-[[1-[(4-fluoro-3-nitro-phenyl)methylsulfonyl]-2,2-dimethyl-4-piperidyl]amino]phenyl]thiophene-2-carboxylate (A-100, 300 mg, 390.47 μmol) in ethanol (5 mL) and water (5 mL) were added iron powder (109.03 mg, 1.95 mmol, 13.87 μL) and ammonium chloride (104.43 mg, 1.95 mmol, 68.26 μL). The suspension was stirred at 75° C. for 4 h. The reaction mixture was filtered through Celite and washed with DCM (10 mL). The filtrate was diluted with water (10 mL) and extracted with DCM (2×10 mL), and the combined organic layer was dried over anhydrous sodium sulfate and filtered. The filtrate was concentrated under reduced pressure and the residue purified by reverse phase column chromatography (30 g, C18-column, compound eluted with 80-85% acetonitrile in 0.1% TFA in water) to afford tert-butyl 5-(3-((1-((3-amino-4-fluorobenzyl)sulfonyl)-2,2-dimethylpiperidin-4-yl)amino)phenyl)-3-(2-(tert-butoxy)-2-oxoethoxy)-4-chlorothiophene-2-carboxylate (A-101a/b), which was subjected to chiral SFC. Purification method: Column Name: YMC Amylose-SA; Flow rate: 5 mL/min; Co-Solvent: 40%; Co-Solvent Name: 0.5% Isopropyl amine in IPA; Outlet Pressure: 100 bar; Injected Volume: 15 μl; Temperature: 35° C. to afford tert-butyl 5-[3-[[(4S)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101a, first eluted fraction, 70 mg, 93.86 μmol, 24% yield). LCMS (ES+): m/z 626.2 [M−2 tBu+H]⁺.

and tert-butyl 5-[3-[[(4R)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101b, second eluted fraction, 70 mg, 93.86 μmol, 24% yield) as an off-white solid. LCMS (ES+): m/z 626.2 [M−2 tBu+H]⁺.

### tert-butyl 5-[3-[[(4S)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105a, First Eluted Fraction) and tert-butyl 5-[3-[[(4R)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105b, Second Eluted Fraction)

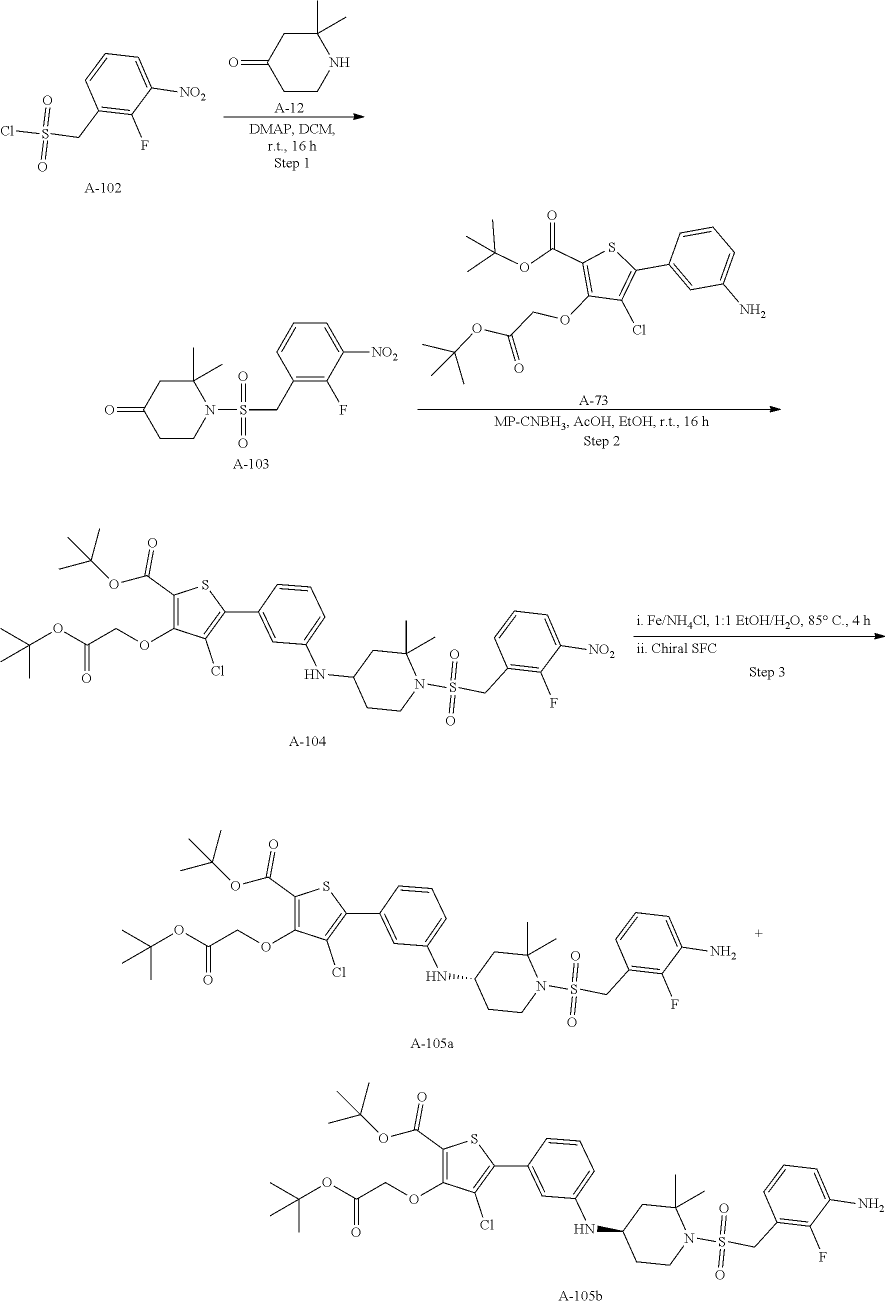

### Step 1: 1-[(2-fluoro-3-nitro-phenyl)methylsulfonyl]-2,2-dimethyl-piperidin-4-one (A-103)

Into a 500 mL single neck round bottom flask containing a well-stirred solution of 2,2-dimethylpiperidin-4-one hydrochloride (A-12, 2 g, 12.22 mmol) and (2-fluoro-3-nitro-phenyl)methane sulfonyl chloride (A-102, 9.30 g, 36.66 mmol) in anhydrous DCM (20 mL) was added DMAP (2.99 g, 24.44 mmol). After 16 h the reaction mixture was diluted with water (250 mL) and extracted with DCM (2×100 mL). The combined organic layer was washed with brine solution (100 mL), dried over sodium sulfate, filtered and concentrated under reduced pressure. The residue was purified by flash silica gel (60-120 mesh) column chromatography (50-60% ethyl acetate in petroleum ether) to afford 1-[(2-fluoro-3-nitro-phenyl)methylsulfonyl]-2,2-dimethyl-piperidin-4-one (A-103, 1.2 g, 3.28 mmol, 27% yield) as a pale yellow solid. LCMS (ES−): m/z 343.2 [M−H]⁻.

### Step 2: tert-butyl 3-(2-tert-butoxy-2-oxo-ethoxy)-4-chloro-5-[3-[[1-[(2-fluoro-3-nitro-phenyl)methylsulfonyl]-2,2-dimethyl-4-piperidyl]amino]phenyl]thiophene-2-carboxylate (A-104)

tert-butyl 3-(2-tert-butoxy-2-oxo-ethoxy)-4-chloro-5-[3-[[1-[(2-fluoro-3-nitro-phenyl)methylsulfonyl]-2,2-dimethyl-4-piperidyl]amino]phenyl]thiophene-2-carboxylate (A-104, 2.56 g, 2.82 mmol, 81% yield) was synthesized from 1-[(2-fluoro-3-nitro-phenyl)methylsulfonyl]-2,2-dimethyl-piperidin-4-one (A-103) and tert-butyl 5-(3-aminophenyl)-3-(2-tert-butoxy-2-oxo-ethoxy)-4-chloro-thiophene-2-carboxylate (A-73) in a similar fashion to Compound A-74a/b, except using 8 eq. MP-cyanoborohydride and 1 eq. Acetic acid. The material was used in next step without purification. LCMS (ESI+) m/z 769.2 [M+H]⁺.

### Step 3: tert-butyl 5-[3-[[(4S)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105a, First Eluted Fraction) and tert-butyl 5-[3-[[(4R)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105b, Second Eluted Fraction)

Into a 250 mL single neck round bottom flask containing a well-stirred solution of tert-butyl 3-(2-tert-butoxy-2-oxo-ethoxy)-4-chloro-5-[3-[[1-[(2-fluoro-3-nitro-phenyl)methylsulfonyl]-2,2-dimethyl-4-piperidyl]amino]phenyl]thiophene-2-carboxylate (A-104, 2.56 g, 3.33 mmol) in a mixture of ethanol (25 mL) and water (15 mL) were added iron powder (930.37 mg, 16.66 mmol) and ammonium chloride (891.16 mg, 16.66 mmol). The resulting suspension was stirred at 85° C. for 4 h. The reaction mixture filtered through Celite, washing with ethanol. The filtrate was concentrated under reduced pressure and the residue was taken up in Ethyl acetate (100 mL) and washed with water (2×50 mL). The organic phase was dried over anhydrous sodium sulfate, filtered and concentrated under reduced pressure. The residue was purified by reverse phase column chromatography [Method: Column: RediSep ISCO C18 (30 g); Mobile phase A: 0.1% TFA in MQ-water; Mobile phase B: Acetonitrile] to afford tert-butyl 5-(3-((1-((3-amino-2-fluorobenzyl)sulfonyl)-2,2-dimethylpiperidin-4-yl)amino)phenyl)-3-(2-(tert-butoxy)-2-oxoethoxy)-4-chlorothiophene-2-carboxylate (A-105a/b), which was subjected to chiral SFC. Method: Chiralcel-OJ-H (250×30 mm) 5.0 μm with Mobile Phase: CO₂: 0.5% Isopropyl amine in methanol (60:40) to afford tert-butyl 5-[3-[[(4S)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105a, first eluted fraction, 0.6 g, 0.813 mmol, 24% yield) and tert-butyl 5-[3-[[(4R)-1-[(3-amino-2-fluoro-phenyl)methylsulfonyl]-2,2-dimethyl-4-piperidylamino]phenyl]-3-(2-tert-butoxy-2-oxo-ethoxy)-4-chloro-thiophene-2-carboxylate (A-105b, second eluted fraction, 1.5 g, 2.032 mmol, 61% yield) as an off-white solid. LCMS (ESI+): m/z 626.1 [M−tBu+H]⁺.

### tert-butyl 5-(3-amino-4-fluorophenyl)-4-chloro-3-(2-ethoxy-2-oxoethoxy)thiophene-2-carboxylate (A-107)

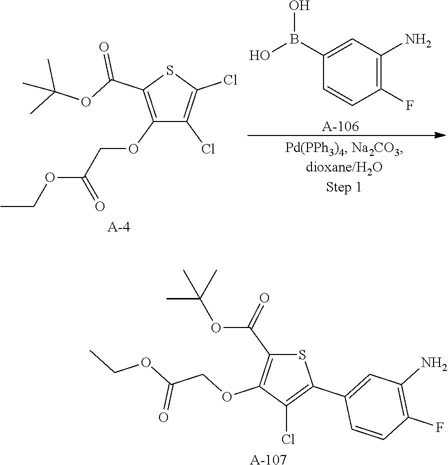

### Step 1: tert-butyl 5-(3-amino-4-fluorophenyl)-4-chloro-3-(2-ethoxy-2-oxoethoxy)thiophene-2-carboxylate (A-107)

A mixture of tert-butyl 4,5-dichloro-3-(2-ethoxy-2-oxoethoxy)thiophene-2-carboxylate (A-4, 10.0 g, 28.1 mmol), (3-amino-4-fluorophenyl)boronic acid (A-106, 5.23 g, 33.8 mmol), Pd(PPh₃)₄ (3.25 g, 2.82 mmol), and Na₂CO₃ (2 M in water, 42.2 mL) in dioxane (150 mL) was purged with N₂ three times, and stirred for 8 h at 75° C. The reaction mixture was acidified to pH 6 using 1N HCl, diluted with water (500 mL) and extracted with ethyl acetate (3×500 mL). The combined organic layers were washed with water (500 mL) and brine (500 mL), dried over sodium sulfate, filtered and concentrated under reduced pressure. The residue was purified by column chromatography (SiO₂, petroleum ether/ethyl acetate=20/1 to 8/1) to afford tert-butyl 5-(3-amino-4-fluorophenyl)-4-chloro-3-(2-ethoxy-2-oxoethoxy)thiophene-2-carboxylate (A-107, 5.95 g, 49% yield) as a yellow solid. LCMS (ES+): 430.1 [M+H]⁺. ¹H NMR (400 MHz, CDCl₃): δ 6.97-7.12 (m, 3H), 4.87-4.91 (m, 2H), 4.25-4.32 (m, 2H), 3.79-3.91 (m, 2H), 1.56-1.59 (m, 9H), 1.29-1.35 (m, 3H).

### 2-[[5-[3-[[1-[(3-aminophenyl)methylsulfonyl]-4-piperidyl]amino]-4-fluoro-phenyl]-2-tert-butoxycarbonyl-4-chloro-3-thienyl]oxy]acetic acid (A-111)

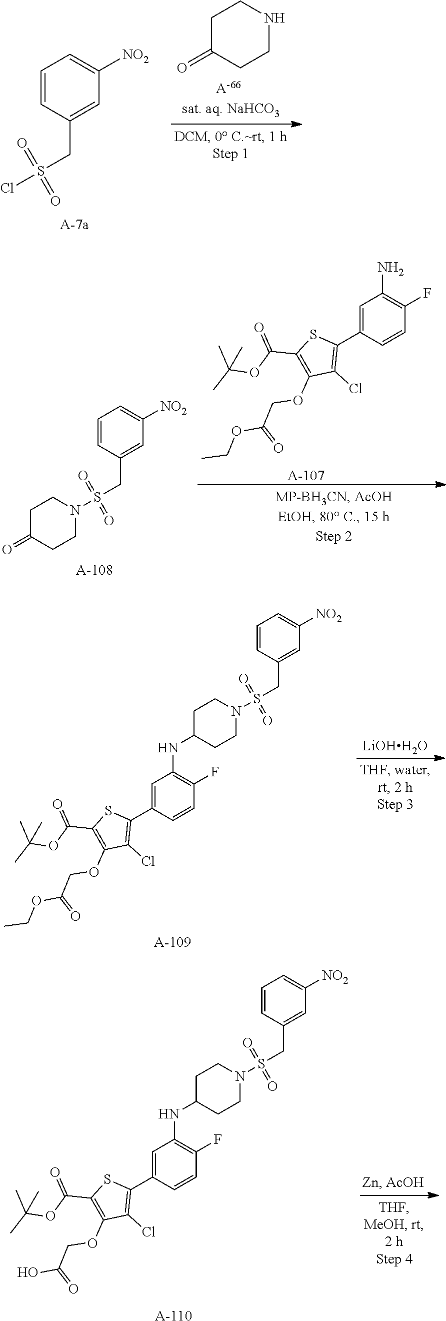

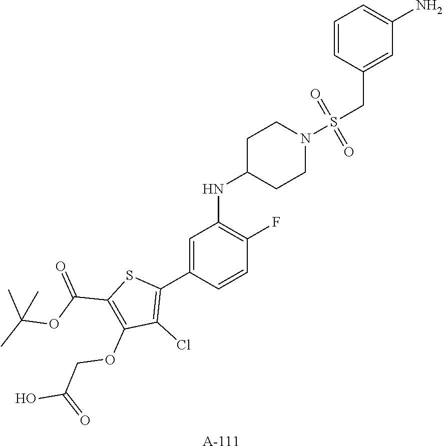 [SMILES: CC(C)(C)OC(=O)c1sc(-c2ccc(F)c(NC3CCN(S(=O)(=O)Cc4cccc(N)c4)CC3)c2)c(Cl)c1OCC(=O)O ; "A-111"]

### Step 1: 1-((3-nitrobenzyl)sulfonyl)piperidin-4-one (A-108)

1-((3-nitrobenzyl)sulfonyl)piperidin-4-one (A-108, 15.5 g, 44.51 mmol, 60% yield) was synthesized from piperidin-4-one hydrochloride (A-66) and (3-nitrophenyl)methanesulfonyl chloride (A-7a) in a similar fashion to Compound A-8, except the product was used in the next step without purification. ¹H NMR (400 MHz, DMSO-d6): δ 8.35 (s, 1H), 8.25 (dd, J=1.60, 8.20 Hz, 1H), 7.91 (d, J=7.60 Hz, 1H), 7.72 (t, J=8.00 Hz, 1H), 4.77 (s, 2H), 3.50 (t, J=6.00 Hz, 4H), 2.40 (t. J=6.00 Hz, 4H).

### Step 2: tert-butyl 4-chloro-3-(2-ethoxy-2-oxoethoxy)-5-(4-fluoro-3-((1-((3-nitrobenzyl)sulfonyl)piperidin-4-yl)amino)phenyl)thiophene-2-carboxylate (A-109)

Into a 100 mL pressure tube containing a well-stirred solution of tert-butyl 5-(3-amino-4-fluorophenyl)-4-chloro-3-(2-ethoxy-2-oxoethoxy)thiophene-2-carboxylate (A-107, 300 mg, 697.85 μmol) in ethanol (20 mL) were added 1-[(3-nitrophenyl)methylsulfonyl]piperidin-4-one (A-108, 1.04 g, 3.49 mmol), acetic acid (209.54 mg, 3.49 mmol, 199.56 μL) and MP-cyanoborohydride (600 mg, 697.85 μmol). The resulting suspension was heated at 80° C. for 15 h. The reaction mixture was cooled to ambient temperature, filtered, the solvent removed under reduced pressure and the residue taken up in ethyl acetate (100 mL). The organic layer was washed with water (75 mL) and brine (75 mL), dried over sodium sulfate and filtered. The solvent was removed under reduced pressure and the residue purified by flash silica gel column chromatography (20-30% of Ethyl acetate in petroleum ether) to afford ter-butyl 4-chloro-3-(2-ethoxy-2-oxoethoxy)-5-(4-fluoro-3-((1-((3-nitrobenzyl)sulfonyl)piperidin-4-yl)amino)phenyl)thiophene-2-carboxylate (A-109, 300 mg, 388.88 μmol, 56% yield) as yellow gummy solid. LCMS (ES+): m/z 655.6 [M−tBu+H]⁺.

### Step 3: 2-[[2-tert-butoxycarbonyl-4-chloro-5-[4-fluoro-3-[[1-[(3-nitrophenyl)methylsulfonyl]-4-piperidyl]amino]phenyl]-3-thienyl]oxy]acetic acid (A-110)

2-[[2-tert-butoxycarbonyl-4-chloro-5-[4-fluoro-3-[[1-[(3-nitrophenyl)methylsulfonyl]-4-piperidyl]amino]phenyl]-3-thienyl]oxy]acetic acid (A-110, 160 mg, 206.11 μmol, 49% yield) was synthesized from tert-butyl 4-chloro-3-(2-ethoxy-2-oxoethoxy)-5-(4-fluoro-3-((1-((3-nitrobenzyl)sulfonyl)piperidin-4-yl)amino)phenyl)thiophene-2-carboxylate (A-109) in a similar fashion to Compound A-9 except using 3 eq. LiOH monohydrate. Upon completion, the reaction mixture was concentrated to almost dryness and the residue acidified to pH˜2 with 1.5 N HCl. The product was extracted into Ethyl acetate (2×). The combined organic layer was washed with brine, dried over sodium sulfate and the solvent removed under reduced pressure. LCMS (ES+): m/z 627.6 [M−tBu+H]⁺.

### Step 4: 2-[[5-[3-[[1-[(3-aminophenyl)methylsulfonyl]-4-piperidyl]amino]-4-fluoro-phenyl]-2-tert-butoxycarbonyl-4-chloro-3-thienyl]oxy]acetic acid (A-111)

2-[[5-[3-[[1-[(3-aminophenyl)methylsulfonyl]-4-piperidyl]amino]-4-fluoro-phenyl]-2-tert-butoxycarbonyl-4-chloro-3-thienyl]oxy]acetic acid (A-111, 100 mg, 128.04 μmol, 55% yield) was synthesized from 2-[[2-tert-butoxycarbonyl-4-chloro-5-[4-fluoro-3-[1-[(3-nitrophenyl)methylsulfonyl]-4-piperidyl]amino]phenyl]-3-thienyl]oxy]acetic acid (A-110) in a similar fashion to Compound A-10, except using 3 eq. zinc powder and 4 eq. Acetic acid. The product was used in the next step without purification. LCMS (ES+): m/z 654.1 [M+H]⁺.

### tert-butyl 5-(3-amino-2-fluorophenyl)-4-chloro-3-(2-ethoxy-2-oxoethoxy)thiophene-2-carboxylate (A-115)

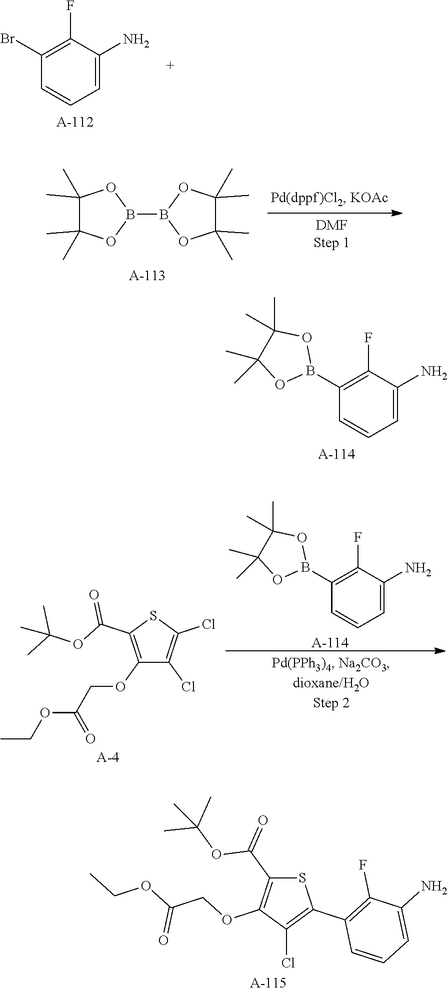

### Step 1: 2-fluoro-3-(4,4,5,5-tetramethyl-1,3,2-dioxaborolan-2-yl)aniline (A-114)

To a solution of 3-bromo-2-fluoroaniline (A-112, 30.0 g, 157 mmol), 4,4,4′,4′,5,5,5′,5′-octamethyl-2,2′-bi(1,3,2-dioxaborolane) (A-113, 48.1 g, 189 mmol) and KOAc (30.0 g, 315 mmol) in DMF (100 mL) was added Pd(dppf)Cl₂ (5.78 g, 7.89 mmol) under N₂ atmosphere. The mixture was stirred at 100° C. for 16 h. The reaction mixture was diluted with water (500 mL) and extracted with ethyl acetate (3×500 mL). The combined organic layers were dried over sodium sulfate, filtered and concentrated under reduced pressure. The residue was purified by column chromatography (SiO₂, petroleum ether/ethyl acetate=20/1 to 4/1) to afford 2-fluoro-3-(4,4,5,5-tetramethyl-1,3,2-dioxaborolan-2-yl)aniline (A-114, 24.2 g, 65% yield) as a white solid.

### Step 2: ter-butyl 5-(3-amino-2-fluorophenyl)-4-chloro-3-(2-ethoxy-2-oxoethoxy)thiophene-2-carboxylate (A-115)

tert-butyl 5-(3-amino-2-fluorophenyl)-4-chloro-3-(2-ethoxy-2-oxoethoxy)thiophene-2-carboxylate (A-115, 7.52 g, 52% yield) was synthesized from tert-butyl 4,5-dichloro-3-(2-ethoxy-2-oxoethoxy)thiophene-2-carboxylate (A-4) and 2-fluoro-3-(4,4,5,5-tetramethyl-1,3,2-dioxaborolan-2-yl)aniline (A-114) in a similar fashion to Compound 107. LCMS (ES+): m/z 374.0 [M-Bu+H]⁺. ¹H NMR (400 MHz, CDCl₃): δ 6.96-7.03 (m, 1H), 6.83-6.90 (m, 1H), 6.77-6.83 (m, 1H), 4.88-4.92 (m, 2H), 4.24-4.32 (m, 2H), 3.77-3.93 (m, 2H), 1.55-1.59 (m, 9H), 1.32 (t, J=7.15 Hz, 3H).

### 2-[[5-[3-[[1-[(3-aminophenyl)methylsulfonyl]-4-piperidyl]amino]-2-fluoro-phenyl]-2-tert-butoxycarbonyl-4-chloro-3-thienyl]oxy]acetic acid (A-118)

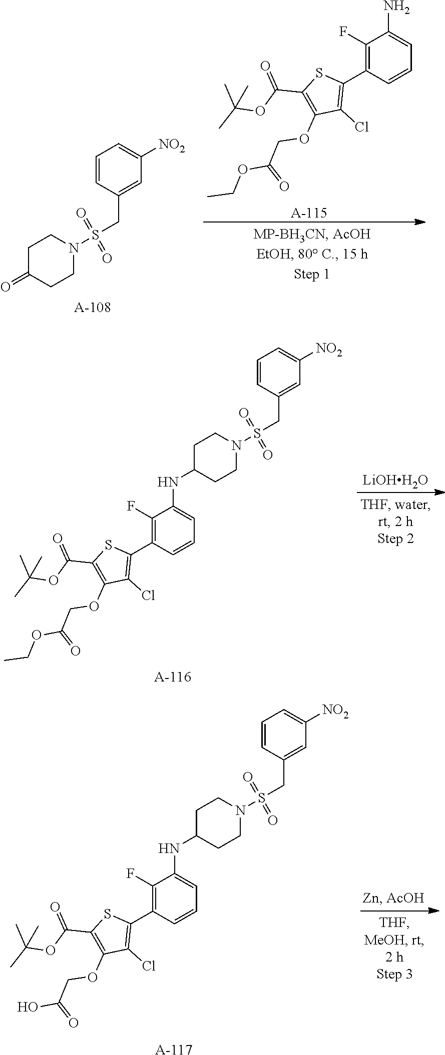

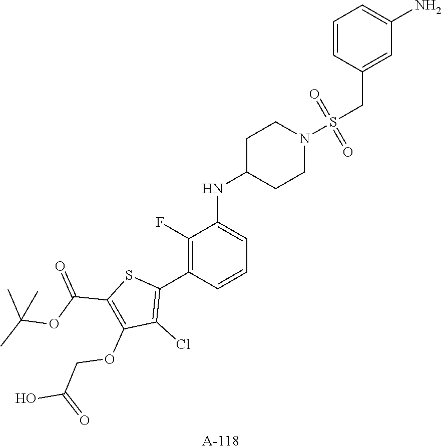 [SMILES: CC(C)(C)OC(=O)c1sc(-c2cccc(NC3CCN(S(=O)(=O)Cc4cccc(N)c4)CC3)c2F)c(Cl)c1OCC(=O)O ; "A-118"]

### Step 1: tert-butyl 4-chloro-3-(2-ethoxy-2-oxo-ethoxy)-5-[2-fluoro-3-[[1-[(3-nitrophenyl)methylsulfonyl]-4-piperidyl]amino]phenyl]thiophene-2-carboxylate (A-116)

Into a 50 mL pressure tube containing a well-stirred solution of tert-butyl-5-(3-amino-2-fluoro-phenyl)-4-chloro-3-(2-ethoxy-2-oxo-ethoxy)thiophene-2-carboxylate (A-115, 500.00 mg, 1.16 mmol) in ethanol (15 mL) were added 1-[(3-nitrophenyl)methylsulfonyl]piperidin-4-one (A-108, 346.97 mg, 1.16 mmol) and acetic acid (279.37 mg, 4.65 mmol, 266.07 μL). The mixture was stirred at 80° C. for 3 h. The reaction mixture was cooled to room temperature and MP-cyanoborohydride (73.09 mg) was added. The reaction was continued at 80° C. for 12 h. The reaction mixture was filtered and washed with ethanol. The solvent was removed under reduced pressure, the residue diluted with water (25 mL) and extracted with ethyl acetate (50 mL). The organic layer was washed with brine (25 mL) and dried over anhydrous sodium sulfate. The solvent was removed under reduced pressure and purified by flash silica gel (230-400 mesh) column chromatography (8-10% of Ethyl acetate in petroleum ether) to afford tert-butyl 4-chloro-3-(2-ethoxy-2-oxo-ethoxy)-5-[2-fluoro-3-[[1-[(3-nitrophenyl)methylsulfonyl]-4-piperidyl]amino]phenyl]thiophene-2-carboxylate (A-116, 600 mg, 782.14 μmol, 67% yield) as a yellow solid. LCMS (ES+): m/z 655.7 [M−tBu+H]⁺.

### Step 2: 2-[[2-tert-butoxycarbonyl-4-chloro-5-[2-fluoro-3-[[1-[(3-nitrophenyl)methylsulfonyl]-4-piperidyl]amino]phenyl]-3-thienyl]oxy]acetic acid (A-117)

2-[[2-tert-butoxycarbonyl-4-chloro-5-[2-fluoro-3-[[1-[(3-nitrophenyl)methylsulfonyl]-4-piperidyl]amino]phenyl]-3-thienyl]oxy]acetic acid (A-117, 500 mg, 594.90 μmol, 77% yield) was synthesized from tert-butyl 4-chloro-3-(2-ethoxy-2-oxo-ethoxy)-5-[2-fluoro-3-[[1-[(3-nitrophenyl]methylsulfonyl]-4-piperidyl]amino]phenyl]thiophene-2-carboxylate (A-116) in a similar fashion to Compound A-9, except using 1 eq. LiOH monohydrate. Upon completion, the reaction mixture was acidified with 1.5N HCl (pH 4-5) and extracted with Ethyl acetate (2×). The organic layer was dried over anhydrous sodium sulfate and solvent was removed under reduced pressure to afford 2-[[2-tert-butoxycarbonyl-4-chloro-5-[2-fluoro-3-[1-[(3-nitrophenyl)methylsulfonyl]-4-piperidyl]amino]phenyl]-3-thienyl]oxy]acetic acid (A-117) as a yellow solid. LCMS (ES+): m/z 628.0 [M−tBu+H]⁺.

### Step 3: 2-[[5-[3-[[1-[(3-aminophenyl)methylsulfonyl]-4-piperidyl]amino]-2-fluoro-phenyl]-2-tert-butoxycarbonyl-4-chloro-3-thienyl]oxy]acetic acid (A-118)

2-[5-[3-[[1-[(3-aminophenyl)methylsulfonyl]-4-piperidyl]amino]-2-fluoro-phenyl]-2-tert-butoxycarbonyl-4-chloro-3-thienyl]oxy]acetic acid (A-118, 170 mg, 228.34 μmol, 38% yield, Formic acid salt) was synthesized from 2-[[2-tert-butoxycarbonyl-4-chloro-5-[2-fluoro-3-[[1-[(3-nitrophenyl)methylsulfonyl]-4-piperidyl]amino]phenyl]-3-thienyl]oxy]acetic acid (A-117) in a similar fashion to Compound A-10, except using 3 eq. zinc powder and 4 eq. Acetic acid. LCMS (ES+): m/z 597.7 [M−tBu+H]⁺.

### 5-(3-((1-((3-(7-aminoheptanamido)benzyl)sulfonyl)piperidin-4-yl)amino)phenyl)-3-(carboxymethoxy)-4-chlorothiophene-2-carboxylic acid hydrochloride (A-121)

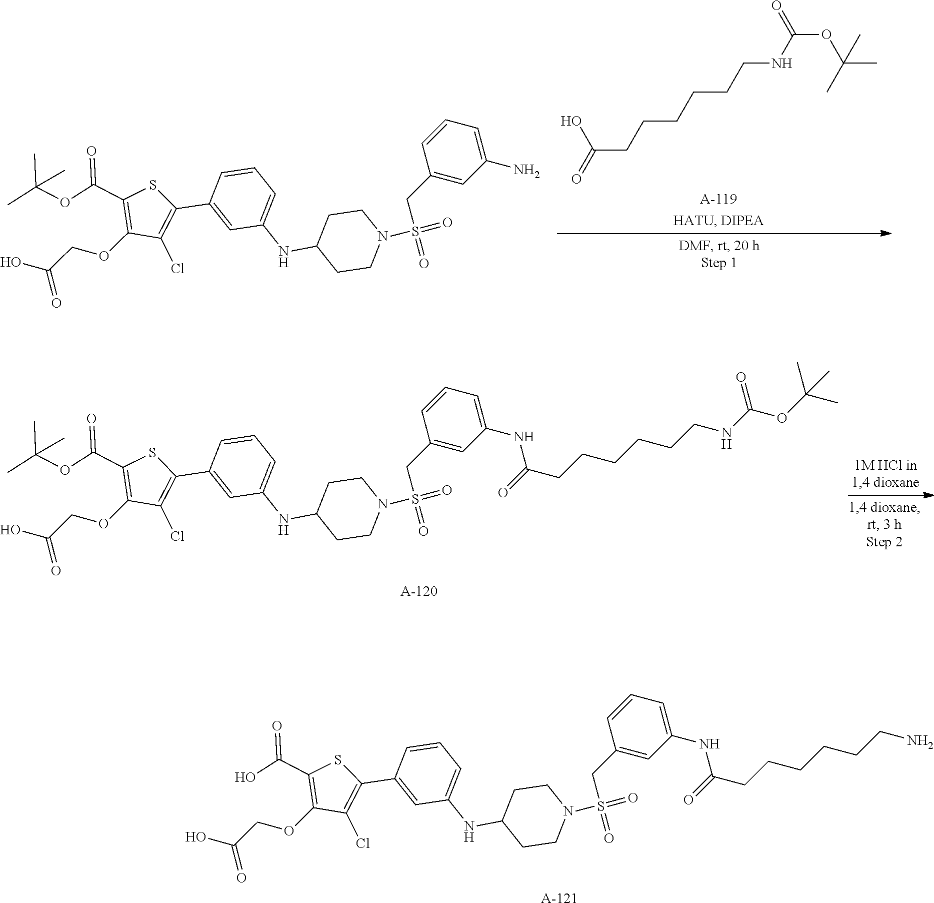

### Step 1: 2-[2-tert-butoxycarbonyl-5-[3-[[1-[[3-[7-(tert-butoxycarbonylamino)heptanoylamino]phenyl]methylsulfonyl]-4-piperidyl]amino]phenyl]-4-chloro-3-thienyl]oxy]acetic acid (A-120)

Into a 10 mL single neck round bottom flask containing a well-stirred solution of 7-(tert-butoxycarbonylamino) heptanoic acid (A-119, 77.12 mg, 314.38 μmol) in DMF (2 mL) were added DIPEA (203.16 mg, 1.57 mmol, 273.79 μL) and HATU (131.49 mg, 345.82 μmol). The reaction mixture was stirred at room temperature for 3 h. Then a solution of 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10, 200 mg, 314.38 μmol) in DMF (1 mL) was added. After 20 h, the solvent was evaporated and the residue was purified by reverse phase preparative HPLC [Purification method: Column: SunFire prep OBD 19×50 mm (5 μm), Mobile phase A: 0.1% TFA in water and Mobile phase B: MeCN] to afford 2-[[2-tert-butoxycarbonyl-5-[3-[[1-[[3-[7-(tert-butoxycarbonylamino)heptanoylamino]phenyl]methylsulfonyl]-4-piperidyl]amino]phenyl]-4-chloro-3-thienyl]oxy]acetic acid (A-120, 140 mg, 159.70 μmol, 51% yield, TFA salt) as a light yellow solid. LCMS (ES+): m/z 863.2 [M+H]⁺.

### Step 2: 5-(3-((1-((3-(7-aminoheptanamido)benzyl)sulfonyl)piperidin-4-yl)amino)phenyl)-3-(carboxymethoxy)-4-chlorothiophene-2-carboxylic acid hydrochloride (A-121)

5-(3-((1-((3-(7-aminoheptanamido)benzyl)sulfonyl)piperidin-4-yl)amino)phenyl)-3-(carboxymethoxy)-4-chlorothiophene-2-carboxylic acid hydrochloride (A-121, 137 mg) was synthesized from 2-[[2-tert-butoxycarbonyl-5-[3-[[1-[[3-[7-(tert-butoxycarbonylamino)heptanoylamino]phenyl]methylsulfonyl]-4-piperidyl]amino]phenyl]-4-chloro-3-thienyl]oxy]acetic acid (A-120) in a similar fashion to Compound A-12. UPLC (ES+): m/z 707.7 [M+H]⁺.

### 5-[3-[[(4S)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-125)

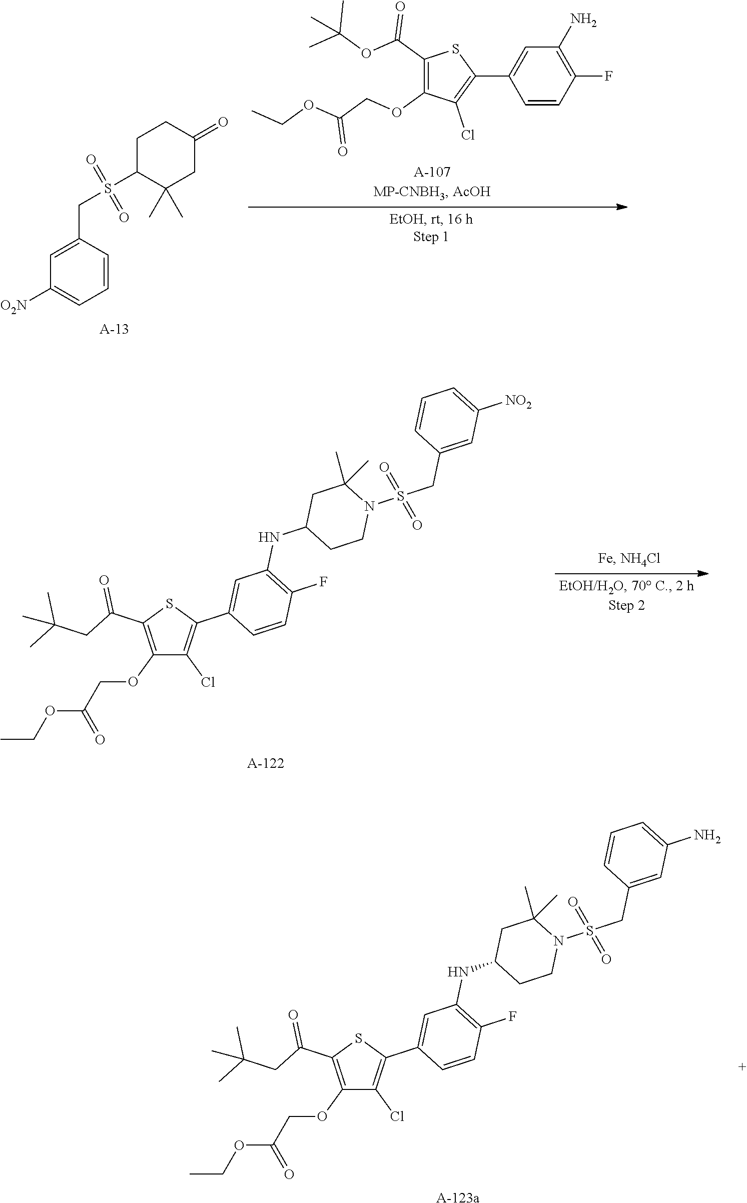

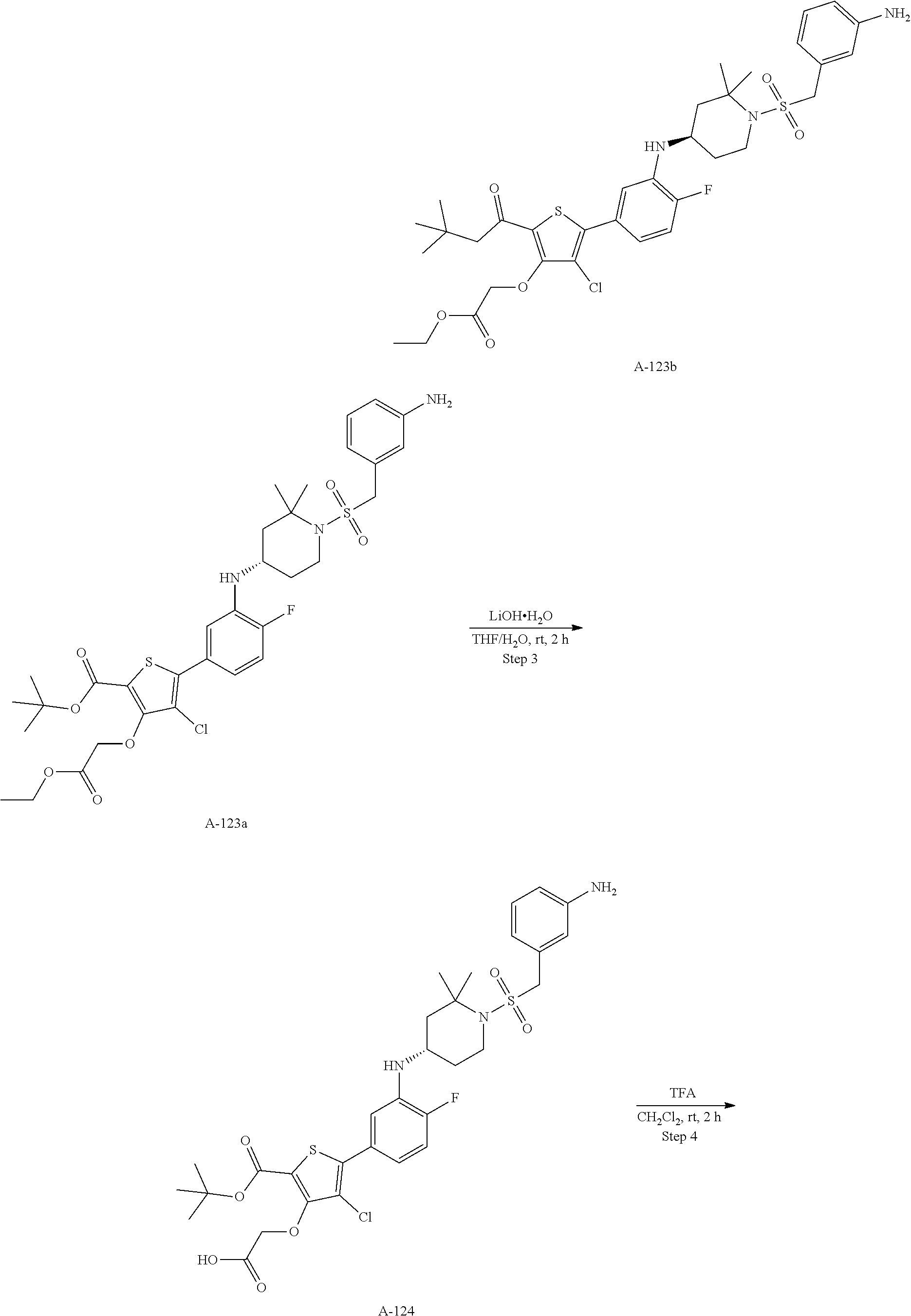 [SMILES: CCOC(=O)COc1c(C(=O)CC(C)(C)C)sc(-c2ccc(F)c(N[C@@H]3CCN(S(=O)(=O)Cc4cccc(N)c4)C(C)(C)C3)c2)c1Cl ; "A-123b" ; CCOC(=O)COc1c(C(=O)OC(C)(C)C)sc(-c2ccc(F)c(N[C@H]3CCN(S(=O)(=O)Cc4cccc(N)c4)C(C)(C)C3)c2)c1Cl ; "LiOH·H2O" ; CC(C)(C)OC(=O)c1sc(-c2ccc(F)c(N[C@H]3CCN(S(=O)(=O)Cc4cccc(N)c4)C(C)(C)C3)c2)c(Cl)c1OCC(=O)O ; "A-124"]

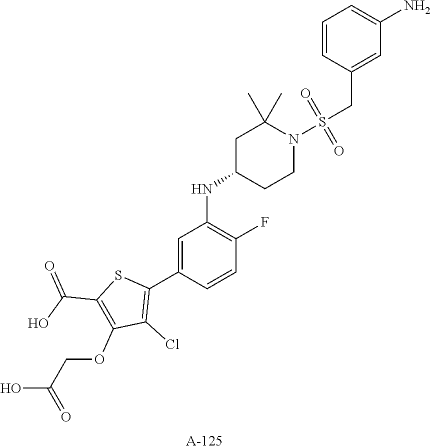 [SMILES: CC1(C)C[C@@H](Nc2cc(-c3sc(C(=O)O)c(OCC(=O)O)c3Cl)ccc2F)CCN1S(=O)(=O)Cc1cccc(N)c1 ; "A-125"]

### Step 1: tert-butyl 4-chloro-5-[3-[[2,2-dimethyl-1-[(3-nitrophenyl)methylsulfonyl]-4-piperidyl]amino]-4-fluoro-phenyl]-3-(2-ethoxy-2-oxo-ethoxy)thiophene-2-carboxylate (A-122)

tert-butyl 4-chloro-5-[3-[[2,2-dimethyl-1-[(3-nitrophenyl)methylsulfonyl]-4-piperidyl]amino]-4-fluoro-phenyl]-3-(2-ethoxy-2-oxo-ethoxy)thiophene-2-carboxylate (A-122, 1.2 g, 1.59 mmol, 68% yield) was synthesized from tert-butyl 5-(3-amino-4-fluorophenyl)-4-chloro-3-(2-ethoxy-2-oxoethoxy)thiophene-2-carboxylate (A-107) and 2,2-dimethyl-1-((3-nitrobenzyl)sulfonyl)piperidin-4-one (A-13) in a similar fashion to Compound A-74a/b, except using 38 eq. Acetic acid. LCMS (ES+): m/z 683.7 [M−tBu+H]⁺.

### Step 2: tert-butyl 5-[3-[[(4S)-1-[(3-aminophenyl)methylsulfonyl]-2,2-dimethyl-4-piperidyl]amino]-4-fluoro-phenyl]-4-chloro-3-(2-ethoxy-2-oxo-ethoxy)thiophene-2-carboxylate (A-123a, First Eluted Fraction) and tert-butyl 5-[3-[[(4R)-1-[(3-aminophenyl)methylsulfonyl]-2,2-dimethyl-4-piperidyl]amino]-4-fluoro-phenyl]-4-chloro-3-(2-ethoxy-2-oxo-ethoxy)thiophene-2-carboxylate (A-123b, Second Eluted Fraction)

Configurations are arbitrarily assigned.

Into a 100 mL single neck round bottom flask containing a well-stirred solution of tert-butyl 4-chloro-5-[3-[[2,2-dimethyl-1-[(3-nitrophenyl)methylsulfonyl]-4-piperidyl]amino]-4-fluoro-phenyl]-3-(2-ethoxy-2-oxo-ethoxy)thiophene-2-carboxylate (A-122, 1.1 g, 1.49 mmol) in ethanol (20 mL) and water (20 mL) were added iron powder (414.96 mg, 7.43 mmol) and ammonium chloride (397.42 mg, 7.43 mmol). The reaction mixture was stirred at 80° C. for 2 h. The reaction mixture was filtered through Celite, the filtrate was diluted with water (20 mL) and extracted with dichloromethane (2×50 mL). The combined organic layer was dried over anhydrous sodium sulfate, filtered and concentrated under reduced pressure. The residue was triturated with diethyl ether, filtered and dried under vacuum to obtain tert-butyl 5-(3-((1-((3-aminobenzyl)sulfonyl)-2,2-dimethylpiperidin-4-yl)amino)-4-fluorophenyl)-4-chloro-3-(2-ethoxy-2-oxoethoxy)thiophene-2-carboxylate (A-123a/b). This material was subjected to chiral SFC to separate isomers. Purification method: Column Name: Chiralcel OJ-H; Flow rate: 5 mL/min; Co-solvent: 50%; Co-solvent Name: 0.5% isopropyl amine in IPA; Outlet Pressure: 100 bar; Injected Volume: 5 μL/min; Temperature: 35° C.] to afford tert-butyl 5-[3-[[(4S)-1-[(3-aminophenyl)methylsulfonyl]-2,2-dimethyl-4-piperidyl]amino]-4-fluoro-phenyl]-4-chloro-3-(2-ethoxy-2-oxo-ethoxy)thiophene-2-carboxylate (A-123a, first eluted fraction, 500 mg, 682.83 μmol, 46% yield) and tert-butyl 5-[3-[[(4R)-1-[(3-aminophenyl)methylsulfonyl]-2,2-dimethyl-4-piperidyl]amino]-4-fluoro-phenyl]-4-chloro-3-(2-ethoxy-2-oxo-ethoxy)thiophene-2-carboxylate (A-123b, second eluted fraction, 500 mg, 647.64 μmol, 44% yield). LCMS (ES−): m/z 709.8 [M−H]⁻.

### Step 3: 2-[[5-[3-[[(4S)-1-[(3-aminophenyl)methylsulfonyl]-2,2-dimethyl-4-piperidyl]amino]-4-fluoro-phenyl]-2-tert-butoxycarbonyl-4-chloro-3-thienyl]oxy]acetic acid (A-124)

2-[[5-[3-[[(4S)-1-[(3-aminophenyl)methylsulfonyl]-2,2-dimethyl-4-piperidyl]amino]-4-fluoro-phenyl]-2-tert-butoxycarbonyl-4-chloro-3-thienyl]oxy]acetic acid (A-124, 460 mg, 648.65 μmol, 92% yield) was synthesized from tert-butyl 5-[3-[[(4S)-1-[(3-aminophenyl)methylsulfonyl]-2,2-dimethyl-4-piperidyl]amino]-4-fluoro-phenyl]-4-chloro-3-(2-ethoxy-2-oxo-ethoxy)thiophene-2-carboxylate (A-123a) in a similar fashion to Compound A-9, except using a 1:1 ratio of THE/water. Upon completion, the reaction mixture was diluted with water and extracted with ethyl acetate (2×). The organic layers were combined, dried over anhydrous sodium sulfate, filtered and concentrated under reduced pressure. LCMS (ES+): m/z 682.8 [M+H]⁺.

### Step 4: 5-[3-[[(4S)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-125)

5-[3-[[(4S)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-125, 130 mg, 167.39 μmol, 76% yield, TFA salt) was synthesized from 2-[[5-[3-[[(4S)-1-[(3-aminophenyl)methylsulfonyl]-2,2-dimethyl-4-piperidyl]amino]-4-fluoro-phenyl]-2-tert-butoxycarbonyl-4-chloro-3-thienyl]oxy]acetic acid (A-124) in a similar fashion to Compound A-26, except using 30 eq. TFA. LCMS (ES−): m/z 625.7 [M−H]⁻.

### 5-[3-[[(4R)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-127)

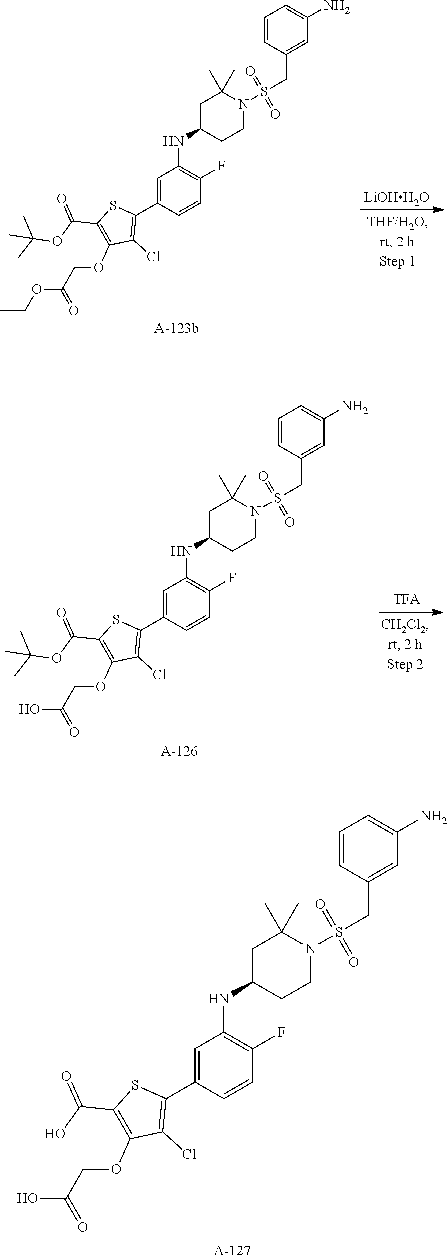

### Step 1: 2-[5-[3-[[(4R)-1-[(3-aminophenyl)methylsulfonyl]-2,2-dimethyl-4-piperidyl]amino]-4-fluoro-phenyl]-2-tert-butoxycarbonyl-4-chloro-3-thienyl]oxy]acetic acid (A-126)

2-[[5-[3-[[(4R)-1-[(3-aminophenyl)methylsulfonyl]-2,2-dimethyl-4-piperidyl]amino]-4-fluoro-phenyl]-2-tert-butoxycarbonyl-4-chloro-3-thienyl]oxy]acetic acid (A-126, 450 mg, 631 μmol, 90% yield) was synthesized from tert-butyl 5-[3-[[(4R)-1-[(3-aminophenyl)methylsulfonyl]-2,2-dimethyl-4-piperidyl]amino]-4-fluoro-phenyl]-4-chloro-3-(2-ethoxy-2-oxo-ethoxy)thiophene-2-carboxylate (A-123b) in a similar fashion to Compound A-9, except using a 1:1 mixture of THF/water. Upon completion, the reaction mixture was diluted with water and extracted with ethyl acetate (2×). The organic layers were combined, dried over anhydrous sodium sulfate, filtered and concentrated under reduced pressure. LCMS (ES+): m/z 682.8 [M+H]⁺.

### Step 2: 5-[3-[[(4R)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-127)

5-[3-[[(4R)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-127, 135 mg, 175 μmol, 80% yield, TFA salt) was synthesized from 2-[[5-[3-[[(4R)-1-[(3-aminophenyl)methylsulfonyl]-2,2-dimethyl-4-piperidyl]amino]-4-fluoro-phenyl]-2-tert-butoxycarbonyl-4-chloro-3-thienyl]oxy]acetic acid (A-126) in a similar fashion to Compound A-26, except using 30 eq. TFA. LCMS (ES−): m/z 625.7 [M−H]⁻.

### 5-[3-[[(4S)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-131)

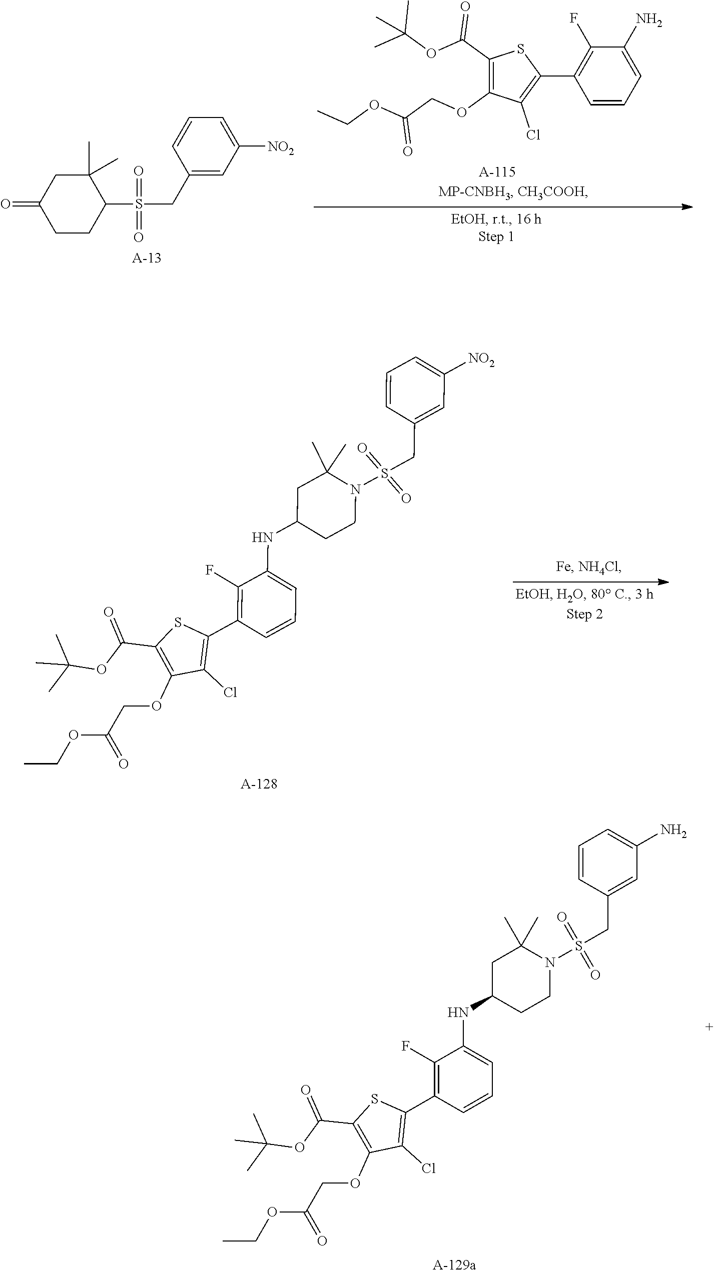

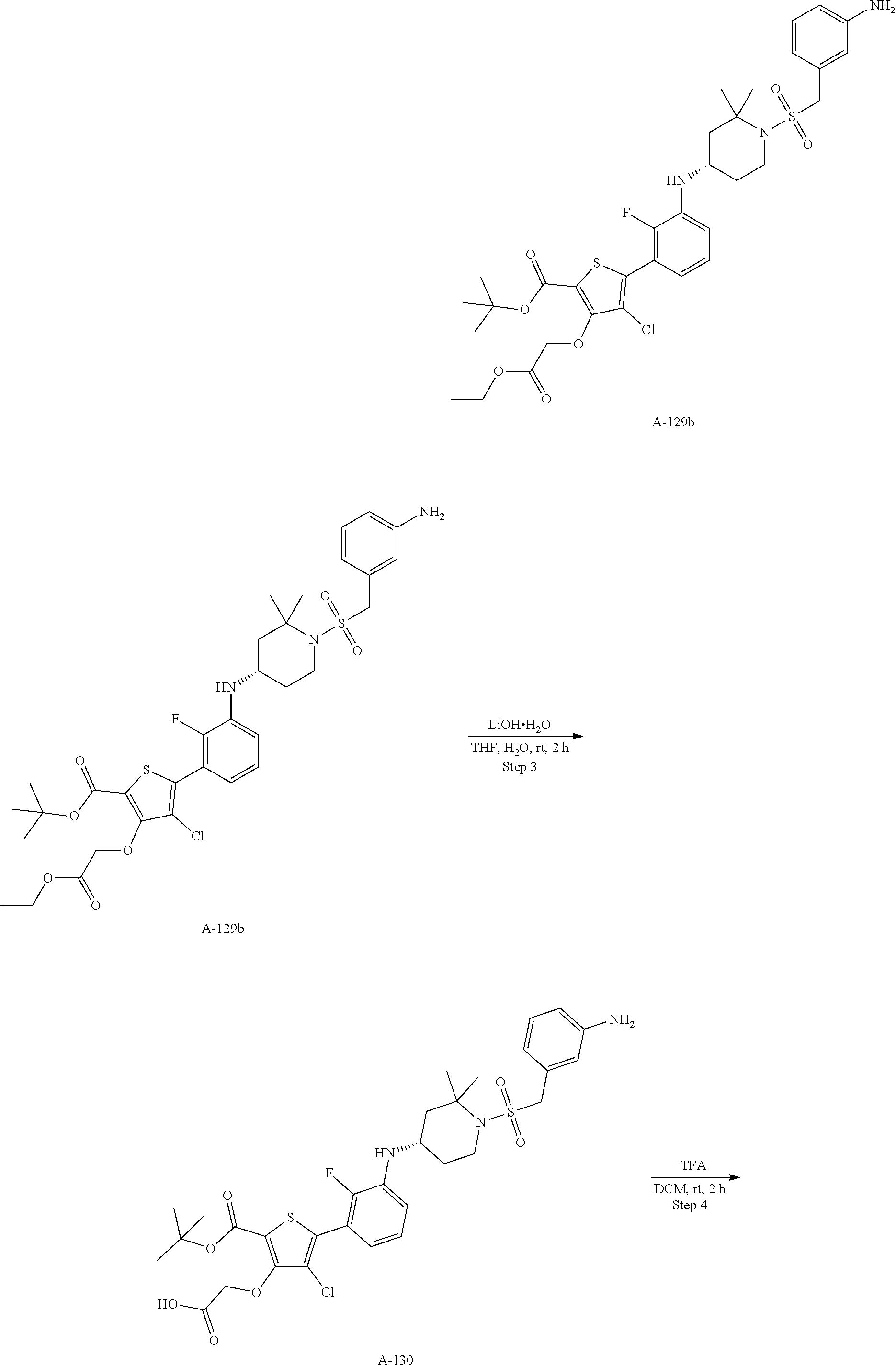 [SMILES: CCOC(=O)COc1c(C(=O)OC(C)(C)C)sc(-c2cccc(N[C@H]3CCN(S(=O)(=O)Cc4cccc(N)c4)C(C)(C)C3)c2F)c1Cl ; CCOC(=O)COc1c(C(=O)OC(C)(C)C)sc(-c2cccc(N[C@H]3CCN(S(=O)(=O)Cc4cccc(N)c4)C(C)(C)C3)c2F)c1Cl ; "LiOH·H2O" ; CC(C)(C)OC(=O)c1sc(-c2cccc(N[C@H]3CCN(S(=O)(=O)Cc4cccc(N)c4)C(C)(C)C3)c2F)c(Cl)c1OCC(=O)O ; "A-130"]

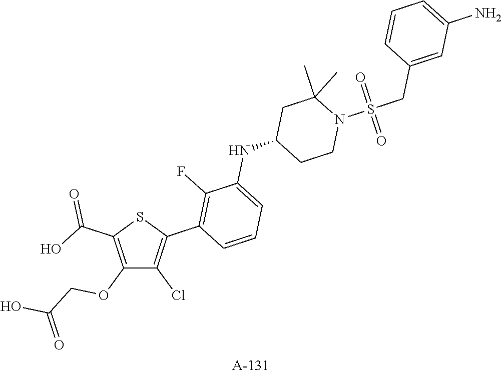 [SMILES: CC1(C)C[C@@H](Nc2cccc(-c3sc(C(=O)O)c(OCC(=O)O)c3Cl)c2F)CCN1S(=O)(=O)Cc1cccc(N)c1 ; "A-131"]

### Step 1: tert-butyl 4-chloro-5-[3-[[2,2-dimethyl-1-[(3-nitrophenyl)methylsulfonyl]-4-piperidyl]amino]-2-fluoro-phenyl]-3-(2-ethoxy-2-oxo-ethoxy)thiophene-2-carboxylate (A-128)

tert-butyl 4-chloro-5-[3-[2,2-dimethyl-1-[(3-nitrophenyl)methylsulfonyl]-4-piperidyl]amino]-2-fluoro-phenyl]-3-(2-ethoxy-2-oxo-ethoxy)thiophene-2-carboxylate (A-128, 1.3 g, 1.58 mmol, 68% yield) was synthesized from tert-butyl 5-(3-amino-2-fluorophenyl)-4-chloro-3-(2-ethoxy-2-oxoethoxy)thiophene-2-carboxylate (A-115) and 2,2-dimethyl-1-[(3-nitrophenyl)methylsulfonyl]piperidin-4-one (A-13B) in a similar fashion to Compound A-74a/b, except using 1.2 eq. A-13, 1.7 eq. MP-cyanoborohydride and 15 eq. acetic acid. LCMS (ES+): m/z 683.7 [M+H]⁺ (tert-butyl cleaved mass).

### Step 2: tert-butyl 5-[3-[[(4R)-1-[(3-aminophenyl)methylsulfonyl]-2,2-dimethyl-4-piperidyl]amino]-2-fluoro-phenyl]-4-chloro-3-(2-ethoxy-2-oxo-ethoxy)thiophene-2-carboxylate (A-129a, First Eluted Fraction) and tert-butyl 5-[3-[[(4S)-1-[(3-aminophenyl)methylsulfonyl]-2,2-dimethyl-4-piperidyl]amino]-2-fluoro-phenyl]-4-chloro-3-(2-ethoxy-2-oxo-ethoxy)thiophene-2-carboxylate (A-129b, Second Eluted Fraction)

Configurations are arbitrarily assigned.

Into a 50 mL single neck round bottom flask containing a well-stirred solution of tert-butyl 4-chloro-5-[3-[[2,2-dimethyl-1-[(3-nitrophenyl)methylsulfonyl]-4-piperidyl]amino]-2-fluoro-phenyl]-3-(2-ethoxy-2-oxo-ethoxy)thiophene-2-carboxylate (A-128, 1.3 g, 1.76 mmol) in a mixture of water (10 mL) and EtOH (10 mL) were added iron powder (490.36 mg, 8.78 mmol) and ammonium chloride (469.69 mg, 8.78 mmol). The reaction mixture was heated at 80° C. for 3 h. The reaction mixture was filtered through Celite, washing with DCM (30 mL). The filtrate was diluted with water (40 mL) and extracted with DCM (3×30 mL). The combined organic layer was dried over anhydrous sodium sulfate, filtered and concentrated under reduced pressure. The residue was purified by flash silica gel column chromatography (50-60% Ethyl acetate in pet-ether) to afford tert-butyl 5-(3-((1-((3-aminobenzyl)sulfonyl)-2,2-dimethylpiperidin-4-yl)amino)-2-fluorophenyl)-4-chloro-3-(2-ethoxy-2-oxoethoxy)thiophene-2-carboxylate (A-129a/b). This material was subjected to chiral SFC [Purification method: Column: Chiralpak OXH, Co-Solvent: 40%; Co-Solvent Name: 0.5% Isopropyl amine in IPA; Outlet Pressure: 100 bar; Injected volume: 10 μl\min; Temperature: 35° C.] to afford tert-butyl 5-[3-[[(4R)-1-[(3-aminophenyl)methylsulfonyl]-2,2-dimethyl-4-piperidyl]amino]-2-fluoro-phenyl]-4-chloro-3-(2-ethoxy-2-oxo-ethoxy)thiophene-2-carboxylate (A-129a, first eluted fraction, 380 mg, 520.72 μmol, 30% yield) as an off-white solid and tert-butyl 5-[3-[[(4S)-1-[(3-aminophenyl)methylsulfonyl]-2,2-dimethyl-4-piperidyl]amino]-2-fluoro-phenyl]-4-chloro-3-(2-ethoxy-2-oxo-ethoxy)thiophene-2-carboxylate (A-129b, second eluted fraction, 290 mg, 401.07 μmol, 23% yield) as an off-white solid. LCMS (ES+): m/z 732.1 [M+Na]⁺.

### Step 3: 2-[5-[3-[[(4S)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-130)

2-[5-[3-[[(4S)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-130, 250 mg, 360.04 μmol, 91% yield) was synthesized from tert-butyl 5-[3-[[(4S)-1-[(3-aminophenyl)methylsulfonyl]-2,2-dimethyl-4-piperidyl]amino]-2-fluoro-phenyl]-4-chloro-3-(2-ethoxy-2-oxo-ethoxy)thiophene-2-carboxylate (A-129b) in a similar fashion to Compound A-9, except using 3 eq. LiOH monohydrate in a 1:1 THF/water mixture. Upon completion, the reaction mixture was diluted with water and extracted with ethyl acetate (2×). The combined organic layer was dried over anhydrous sodium sulfate, filtered and concentrated under reduced pressure. LCMS (ES−): m/z 679.8 [M−H]⁻.

### Step 4: 5-[3-[(49)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-131)

5-[3-[[(4S)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-131, 140 mg, 178.64 μmol, 81% yield, TFA salt) was synthesized from 2-[5-[3-[[(4S)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-130) in a similar fashion to Compound A-26, except using 59 eq. TFA. The residue was azeotroped with toluene (2×). LCMS (ES+): m/z 626.2 [M+H]⁺.

### 5-[3-[[(4R)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-133)

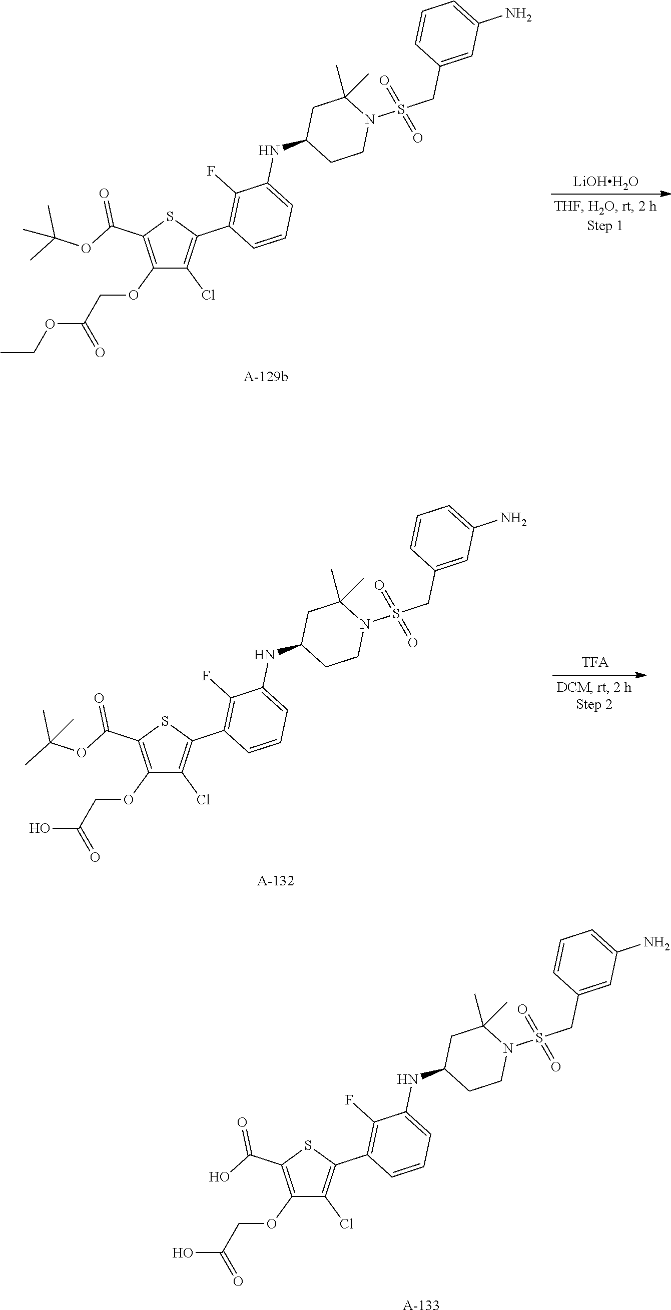 [SMILES: CCOC(=O)COc1c(C(=O)OC(C)(C)C)sc(-c2cccc(N[C@@H]3CCN(S(=O)(=O)Cc4cccc(N)c4)C(C)(C)C3)c2F)c1Cl ; "LiOH·H2O" ; CC(C)(C)OC(=O)c1sc(-c2cccc(N[C@@H]3CCN(S(=O)(=O)Cc4cccc(N)c4)C(C)(C)C3)c2F)c(Cl)c1OCC(=O)O ; "A-132" ; CC1(C)C[C@H](Nc2cccc(-c3sc(C(=O)O)c(OCC(=O)O)c3Cl)c2F)CCN1S(=O)(=O)Cc1cccc(N)c1 ; "A-133"]

### Step 1: 2-[[5-[3-[[(4R)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-132)

2-[[5-[3-[[(4R)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-133, 340 mg, 0.491 mmol, 94% yield) was synthesized from tert-butyl 5-[3-[[(4R)-1-[(3-aminophenyl)methylsulfonyl]-2,2-dimethyl-4-piperidyl]amino]-2-fluoro-phenyl]-4-chloro-3-(2-ethoxy-2-oxo-ethoxy)thiophene-2-carboxylate (A-129a) in a similar fashion to Compound A-9, except using 3 eq. LiOH monohydrate in a 1:1 THF/water mixture. Upon completion, the reaction mixture was diluted with water and extracted with ethyl acetate (2×). The combined organic layer was dried over anhydrous sodium sulfate, filtered and concentrated under reduced pressure. LCMS (ES−): m/z 680.1 [M−H]⁻.

### Step 2: 5-[3-[[(4R)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-133)

5-[3-[[(4R)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-133, 140 mg, 0.174 mmol, 79% yield, TFA salt) was synthesized from 2-[[5-[3-[[(4R)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-132) in a similar fashion to Compound A-26, except using 59 equiv. TFA. The residue was azeotroped with toluene (2×). LCMS (ES+): m/z 626.7 [M+H]⁺.
5-[3-[[1-[[3-(2-aminoethylamino)phenyl]methylsulfonyl]-4-piperidyl]amino]phenyl]-3-(carboxymethoxy)-4-chloro-thiophene-2-carboxylic acid (A-136)

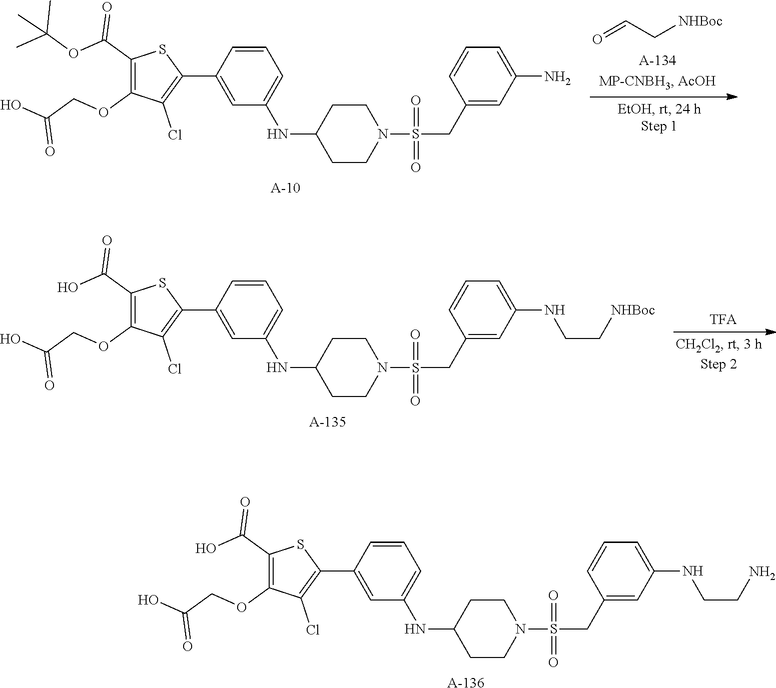

### Step 1: tert-butyl 5-[3-[[1-[[3-[2-(tert-butoxycarbonylamino)ethylamino]phenyl]methylsulfonyl]-4-piperidyl]amino]phenyl]-3-(2-tert-butoxy-2-oxo-ethoxy)-4-chloro-thiophene-2-carboxylate (A-135)

tert-butyl 5-[3-[[1-[[3-[2-(tert-butoxycarbonylamino)ethylamino]phenyl]methylsulfonyl]-4-piperidyl]amino]phenyl]-3-(2-tert-butoxy-2-oxo-ethoxy)-4-chloro-thiophene-2-carboxylate (A-135, 350 mg, 273.85 μmol, 63% yield) was synthesized from tert-butyl 5-[3-[[1-[(3-aminophenyl)methylsulfonyl]-4-piperidyl]amino]phenyl]-3-(2-tert-butoxy-2-oxo-ethoxy)-4-chloro-thiophene-2-carboxylate (A-10) and tert-butyl N-(2-oxoethyl) carbamate (A-134) in a similar fashion to Compound A-74a/b, except using 2.3 eq. MP-cyanoborohydride. The material was which was triturated with diethyl ether and used in the next step without further purification. LCMS (ES+): m/z 835.2 [M+H]⁺.

### Step 2: 5-[3-[[1-[[3-(2-aminoethylamino)phenyl]methylsulfonyl]-4-piperidyl]amino]phenyl]-3-(carboxymethoxy)-4-chloro-thiophene-2-carboxylic acid (A-136)

5-[3-[[1-[[3-(2-aminoethylamino)phenyl]methylsulfonyl]-4-piperidyl]amino]phenyl]-3-(carboxymethoxy)-4-chloro-thiophene-2-carboxylic acid (A-136, 160 mg, 184.67 μmol, 36% yield, TFA salt) was synthesized from tert-butyl 5-[3-[[1-[[3-[2-(tert-butoxycarbonylamino)ethylamino]phenyl]methylsulfonyl]-4-piperidyl]amino]phenyl]-3-(2-tert-butoxy-2-oxo-ethoxy)-4-chloro-thiophene-2-carboxylate (A-135) in a similar fashion to Compound A-62, except using 67 eq. TFA. The material was triturated with diethyl ether. LCMS (ES+): m/z 623.1 [M+H]⁺.

### 5-[3-[[(4S)-1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-139)

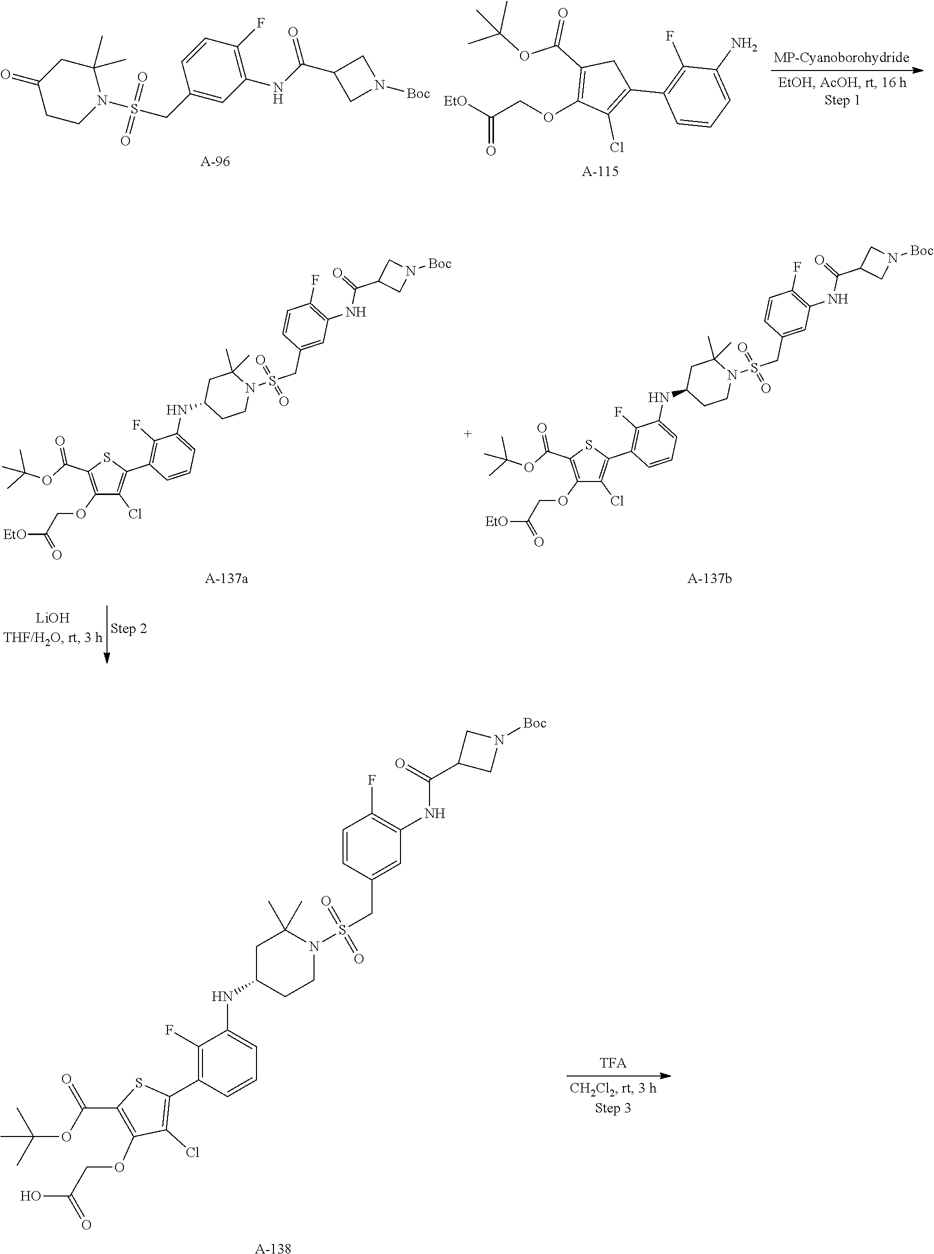

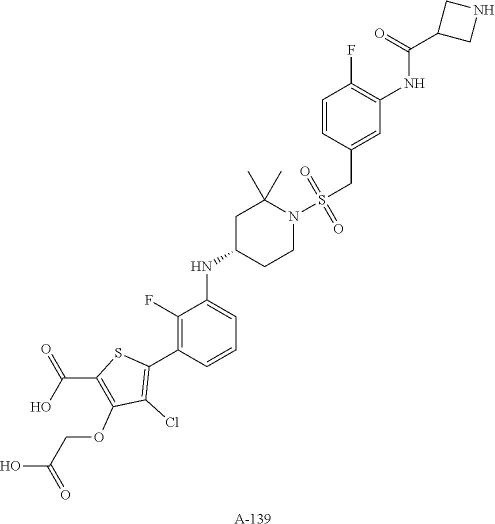 [SMILES: CC1(C)C[C@@H](Nc2cccc(-c3sc(C(=O)O)c(OCC(=O)O)c3Cl)c2F)CCN1S(=O)(=O)Cc1ccc(F)c(NC(=O)C2CNC2)c1 ; "A-139"]

### Step 1: tert-butyl 3-[[5-[[(4S)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137a, First Eluted Fraction) and tert-butyl 3-[[5-[[(4R)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137b, Second Eluted Fraction)

Configurations are arbitrarily assigned.

tert-butyl 3-[[5-[[(4)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137a/b, 900 mg, 96.5%) was synthesized from tert-butyl 3-[[5-[(2,2-dimethyl-4-oxo-1-piperidyl)sulfonylmethyl]-2-fluoro-phenyl]carbamoyl]azetidine-1-carboxylate (A-96) and tert-butyl 5-(3-amino-2-fluorophenyl)-4-chloro-3-(2-ethoxy-2-oxoethoxy)thiophene-2-carboxylate (A-115) in a similar fashion to Compound A-74a/b, except using 1.2 eq. A-115, 1.7 eq. MP-cyanoborohydride and 1 mL acetic acid. LCMS (ES−): m/z 909.2 [M−H]⁻.

A-137a/b was subjected to chiral SFC to separate isomers [Purification method: Column Name: Lux A1; Flow rate: 4 mL/min; Co-Solvent: 30%; Co-Solvent Name: IPA; Outlet Pressure: 100 bar; Injected Volume: 6 μL\min; Temperature: 35° C.] to afford tert-butyl 3-[5-[[(4S)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137a, first eluted fraction, 380 mg, 416.91 μmol, 36% yield) and tert-butyl 3-[[5-[[(4R)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137b, second eluted fraction, 340 mg, 369.29 μmol, 32% yield).

### Step 2: 2-[[2-tert-butoxycarbonyl-5-[3-[[(4S)-1-[[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-138)

2-[[2-tert-butoxycarbonyl-5-[3-[[(4S)-1-[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-138, 320 mg, 328.91 μmol, 79% yield) was synthesized from tert-butyl 3-[[5-[[(4S)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137a) in a similar fashion to Compound A-9, except using 3 eq. LiOH monohydrate in a 1:1 mixture of THE/water. Upon completion, the mixture was diluted with water and extracted with Ethyl acetate (2×). The organic layers were combined, dried over anhydrous sodium sulfate, filtered and concentrated under reduced pressure. LCMS (ES−): m/z 883.42 [M−H]⁻.

### Step 3: 5-[3-[[(4S)-1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-139)

5-[3-[[(4S)-1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-139, 295 mg, 324.14 μmol, 99% yield, TFA salt) was synthesized from 2-[[2-tert-butoxycarbonyl-5-[3-[[(4S)-1-[[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-138) in a similar fashion to Compound A-62, except using 5 eq. TFA. The material was triturated with diethyl ether. LCMS (ES+): m/z 727.1 [M+H]⁺.

### 5-[3-[[1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-141)

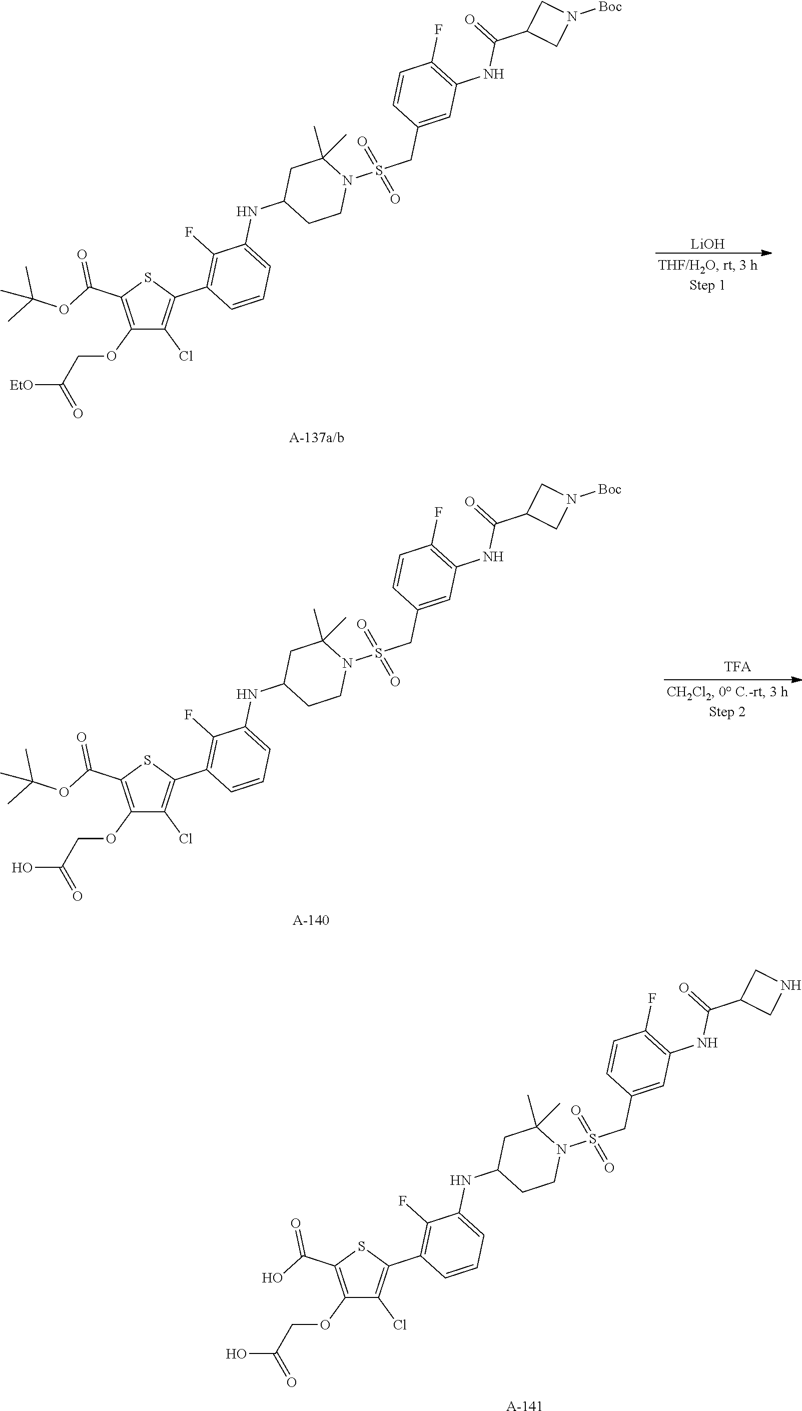 [SMILES: CCOC(=O)COc1c(C(=O)OC(C)(C)C)sc(-c2cccc(NC3CCN(S(=O)(=O)Cc4ccc(F)c(NC(=O)C5CN(C(=O)OC(C)(C)C)C5)c4)C(C)(C)C3)c2F)c1Cl ; "LiOH" ; CC(C)(C)OC(=O)c1sc(-c2cccc(NC3CCN(S(=O)(=O)Cc4ccc(F)c(NC(=O)C5CN(C(=O)OC(C)(C)C)C5)c4)C(C)(C)C3)c2F)c(Cl)c1OCC(=O)O ; "A-140" ; CC1(C)CC(Nc2cccc(-c3sc(C(=O)O)c(OCC(=O)O)c3Cl)c2F)CCN1S(=O)(=O)Cc1ccc(F)c(NC(=O)C2CNC2)c1 ; "A-141"]

### Step 1: 2-[[2-tert-butoxycarbonyl-5-[3-[[1-[[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-140)

2-[[2-tert-butoxycarbonyl-5-[3-[[1-[[3-[(1-tert-butoxycarbonylazetidine-3-carbonyl)amino]-4-fluoro-phenyl methylsulfonyl]-2,2-dimethyl-4-piperidyl]amino]-2-fluoro-phenyl]-4-chloro-3-thienyl]oxy]acetic acid (A-140, 110 mg, 92.14 μmol, 65% yield) was synthesized from tert-butyl 3-[[5-[[4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137a/b) in a similar fashion to Compound A-9, except using 3 eq. LiOH monohydrate in a 1:1 THF/water mixture. Upon completion, the reaction mixture was diluted with water and extracted with ethyl acetate (2×). The organic layers were combined, dried over anhydrous sodium sulfate, filtered and concentrated under reduced pressure. LCMS (ES−): m/z 881.2 [M−H]⁻.

### Step 2: 5-[3-[[1-[[3-(azetidine-3-carbonylamino)-4-fluoro-phenylmethylsulfonyl]-2,2-dimethyl-4-piperidyl]amino]-2-fluoro-phenyl]-3-(carboxymethoxy)-4-chloro-thiophene-2-carboxylic acid (A-141)

5-[3-[[1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-141) was synthesized from 2-[[2-tert-butoxycarbonyl-5-[3-[[1-[[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-140) in a similar fashion to Compound A-62, except using 5 eq. TFA. The material was triturated with diethyl ether. LCMS (ES+): m/z 727.0 [M+H]⁺.

### 5-[3-[[(4R)-1-[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-143)

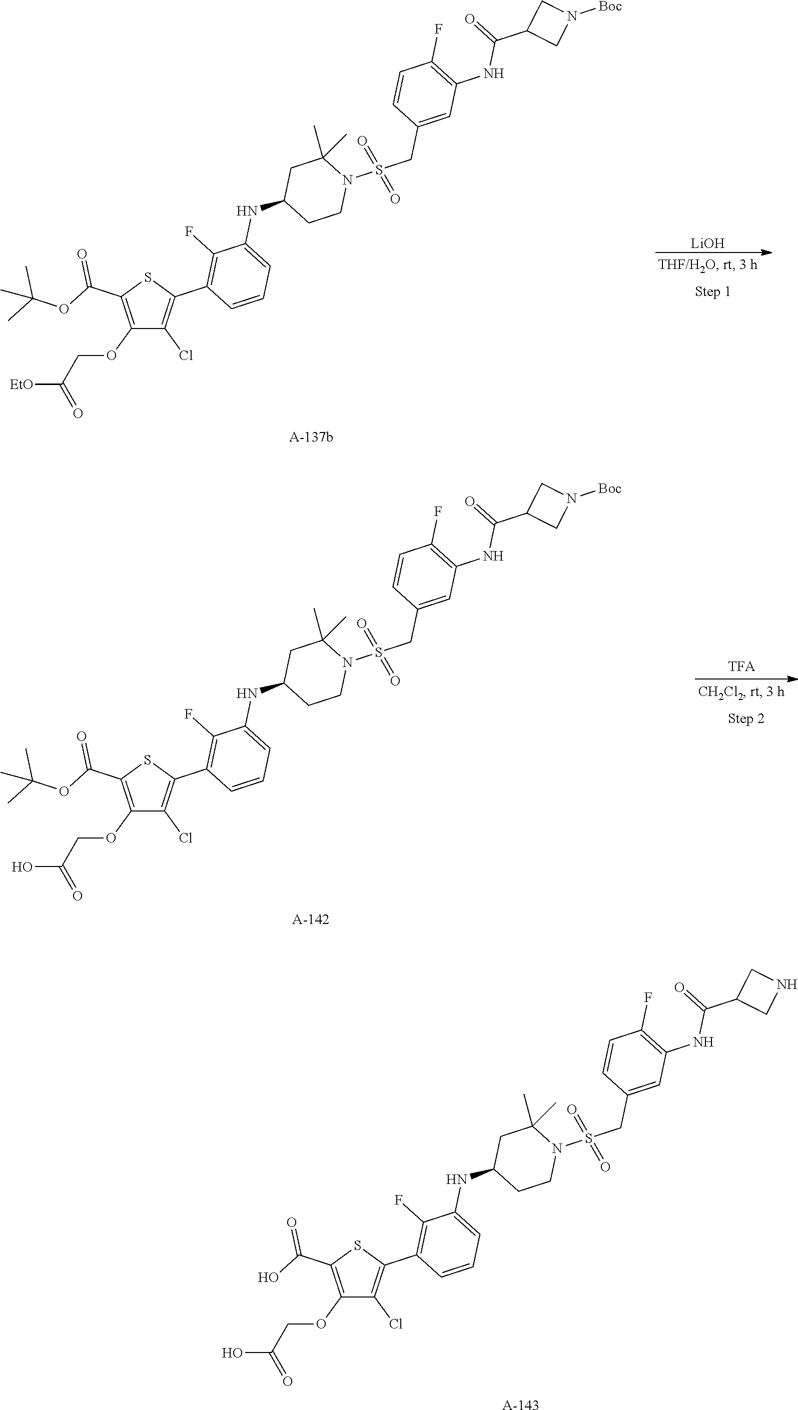 [SMILES: CCOC(=O)COc1c(C(=O)OC(C)(C)C)sc(-c2cccc(N[C@@H]3CCN(S(=O)(=O)Cc4ccc(F)c(NC(=O)C5CN(C(=O)OC(C)(C)C)C5)c4)C(C)(C)C3)c2F)c1Cl ; "LiOH" ; CC(C)(C)OC(=O)c1sc(-c2cccc(N[C@@H]3CCN(S(=O)(=O)Cc4ccc(F)c(NC(=O)C5CN(C(=O)OC(C)(C)C)C5)c4)C(C)(C)C3)c2F)c(Cl)c1OCC(=O)O ; "A-142" ; CC1(C)C[C@H](Nc2cccc(-c3sc(C(=O)O)c(OCC(=O)O)c3Cl)c2F)CCN1S(=O)(=O)Cc1ccc(F)c(NC(=O)C2CNC2)c1 ; "A-143"]

### Step 1: 2-[2-tert-butoxycarbonyl-5-[3-[[(4R)-1-[[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-142)

2-[[2-tert-butoxycarbonyl-5-[3-[[(4R)-1-[3-[(1-tert-butoxycarbonylazetidine-3-carbonyl)amino]-4-fluoro-phenylmethylsulfonyl]-2,2-dimethyl-4-piperidyl]amino]-2-fluoro-phenyl]-4-chloro-3-thienyl]oxy]acetic acid (A-142, 280 mg, 71% yield) was synthesized from tert-butyl 3-[[5-[(4R)-4-[3-[5-tert-butoxycarbonyl-3-chloro-4-(2-ethoxy-2-oxo-ethoxy)-2-thienyl]-2-fluoro-anilino]-2,2-dimethyl-1-piperidyl]sulfonylmethyl]-2-fluoro-phenyl]carbamoyl]azetidine-1-carboxylate (A-137b) in a similar fashion to Compound A-9, except using 3 eq. LiOH monohydrate in a 1:1 THF/water mixture. Upon completion, the reaction mixture was diluted with water and extracted with ethyl acetate (2×). The combined organic layers were washed with brine (10 mL) and dried over anhydrous sodium sulfate, filtered and concentrated under reduced pressure. LCMS (ES+): m/z 783.2 [M−Boc+H]⁺.

### Step 2: 5-[3-[[(4R)-1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-143)

5-[3-[[(4R)-1-[3-(azetidine-3-carbonylamino)-4-fluoro-phenylmethylsulfonyl]-2,2-dimethyl-4-piperidyl]amino]-2-fluoro-phenyl]-3-(carboxymethoxy)-4-chloro-thiophene-2-carboxylic acid (A-143, 250 mg, 96% yield, TFA salt) was synthesized from 2-[2-tert-butoxycarbonyl-5-[3-[[(4R)-1-[3-[(1-tert-butoxycarbonylazetidine-3-carbonyl)amino]-4-fluoro-phenyl]methylsulfonyl]-2,2-dimethyl-4-piperidyl]amino]-2-fluoro-phenyl]-4-chloro-3-thienyl]oxy]acetic acid (A-142) in a similar fashion to Compound A-62, except using 1 eq. TFA. The material was triturated with diethyl ether. LCMS (ES+): m/z 727.0 [M+H]⁺.

3-[2-[2-[3-[[2-(2,6-dioxo-3-piperidyl)-1,3-dioxo-isoindolin-4-yl]propoxy]ethoxy]ethoxy]propanoic acid (B-7)

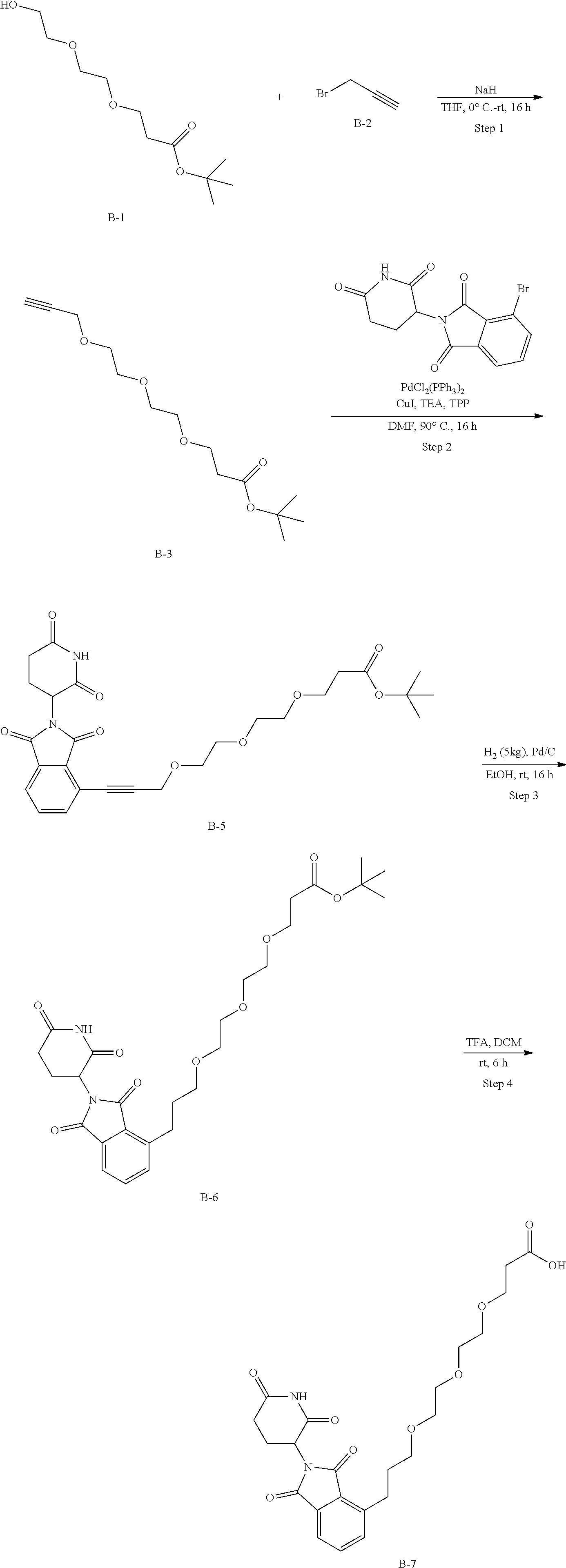

### Step 1: tert-butyl 3-[2-(2-prop-2-ynoxyethoxy)ethoxy]propanoate (B-3)

Into a 100 mL two neck round bottom flask containing a well-stirred suspension of tert-butyl 3-[2-(2-hydroxyethoxy)ethoxy]propanoate (B-1, 1 g, 4.27 mmol) in THE (20 mL) was added sodium hydride (60% dispersion in mineral oil) (88.31 mg, 3.84 mmol) and 3-bromoprop-1-yne (B-2, 507.75 mg, 4.27 mmol) at 0° C. The reaction mixture was stirred at room temperature for 16 h. The reaction was quenched with water (10 mL) and the volatiles were removed under reduced pressure. The residue was diluted with Ethyl acetate (200 mL) and washed with water (2×150 mL). The Ethyl acetate layer was dried over anhydrous sodium sulfate and solvent was removed under reduced pressure. The residue was purified by flash silica gel column chromatography (40% Ethyl acetate in petroleum ether) to afford tert-butyl 3-[2-(2-prop-2-ynoxyethoxy)ethoxy]propanoate (B-3, 340 mg, 1.25 mmol, 29% yield) as a pale yellow liquid. ¹H NMR (400 MHz, DMSO-d₆): δ 4.13 (t, J=2.32 Hz, 2H), 3.59-3.32 (m, 11H), 2.41 (t. J=6.20 Hz, 2H), 1.39 (s, 9H).

### Step 2: tert-butyl 3-[2-[2-[3-[[2-(2,6-dioxo-3-piperidyl)-1,3-dioxo-isoindolin-4-yl]prop-2-ynoxy]ethoxy]ethoxy]propanoate (B-5)

Into a 25 mL pressure tube containing a well-stirred solution of 4-bromo-2-(2,6-dioxo-3-piperidyl)isoindoline-1,3-dione (B-4, 340 mg, 1.01 mmol) and tert-butyl 3-[2-(2-prop-2-ynoxyethoxy)ethoxy]propanoate (B-3, 329.59 mg, 1.21 mmol) in DMF (5 mL) was added triethylamine (1.53 g, 15.13 mmol, 2.11 mL) at room temperature. The reaction mixture was purged with nitrogen gas for 15 min. Then PdCl₂(PPh₃) (70.79 mg, 100.85 μmol), copper (I) iodide (38.41 mg, 201.71 μmol, 6.84 μL) and triphenylphosphine (26.45 mg, 100.85 μmol) were added and purged with nitrogen gas for another 5 min. The tube was sealed, and the reaction mixture was stirred at 90° C. for 16 h. The mixture was cooled to room temperature, diluted with Ethyl acetate (40 mL) and washed with water (2×30 mL). The Ethyl acetate layer was dried over anhydrous sodium sulfate and solvent was removed under reduced pressure. The residue was purified by flash silica gel column chromatography (40% Ethyl acetate in petroleum ether) to obtain tert-butyl 3-[2-[2-[3-[[2-(2,6-dioxo-3-piperidyl)-1,3-dioxo-isoindolin-4-yl]prop-2-ynoxy]ethoxy]ethoxy]propanoate (B-5, 240 mg, 354.18 μmol, 35% yield) as a pale yellow solid. LCMS (ES−): m/z 527.6 [M−H]⁻.

### Step 3: tert-butyl 3-[2-[2-[3-[[2-(2,6-dioxo-3-piperidyl)-1,3-dioxo-isoindolin-4-yl]propoxy]ethoxy]ethoxy]propanoate (B-6)

In to a 100 mL tiny clave containing a well-stirred solution of tert-butyl 3-[2-[2-[3-[[2-(2,6-dioxo-3-piperidyl)-1,3-dioxo-isoindolin-4-yl]prop-2-ynoxy]ethoxy]ethoxy]propanoate (B-5, 240 mg, 454.07 μmol) in ethanol (15 mL) was added palladium (10% on carbon) (106.31 mg, 99.9 μmol) under nitrogen atmosphere. The reaction mixture was then hydrogenated under 5 kg pressure for 16 h. The reaction mixture was filtered through Celite, washed with ethanol (20 mL) and the filtrate concentrated under reduced pressure. The residue was purified by flash silica gel column chromatography (40% Ethyl acetate in petroleum ether) to obtain tert-butyl 3-[2-[2-[3-[[2-(2,6-dioxo-3-piperidyl)-1,3-dioxo-isoindolin-4-yl]propoxy]ethoxy]ethoxy]propanoate (B-6, 130 mg, 244.09 μmol, 54% yield) as a pale yellow solid. LCMS (ES−): m/z 531.6 [M−H]⁻.

### Step 4: 3-[2-[2-[3-[[2-(2,6-dioxo-3-piperidyl)-1,3-dioxo-isoindolin-4-yl]propoxy]ethoxy]ethoxy]propanoic acid (B-7)

3-[2-[2-[3-[[2-(2,6-dioxo-3-piperidyl)-1,3-dioxo-isoindolin-4-yl]propoxy]ethoxy]ethoxy]propanoic acid (B-7, 90 mg, 179.44 μmol, 35% yield, TFA salt) was synthesized from tert-butyl 3-[2-[2-[3-[[2-(2,6-dioxo-3-piperidyl)-1,3-dioxo-isoindolin-4-yl]propoxy]ethoxy]ethoxy]propanoate (B-6) in a similar fashion to Compound A-26, except using 5 eq. TFA. LCMS (ES+): m/z 477.5 [M+H]⁺.

### 7-[2-[[2-(2,6-dioxo-3-piperidyl)-1,3-dioxo-isoindolin-4-yl]amino]acetyl]amino]heptanoic acid (B-11)

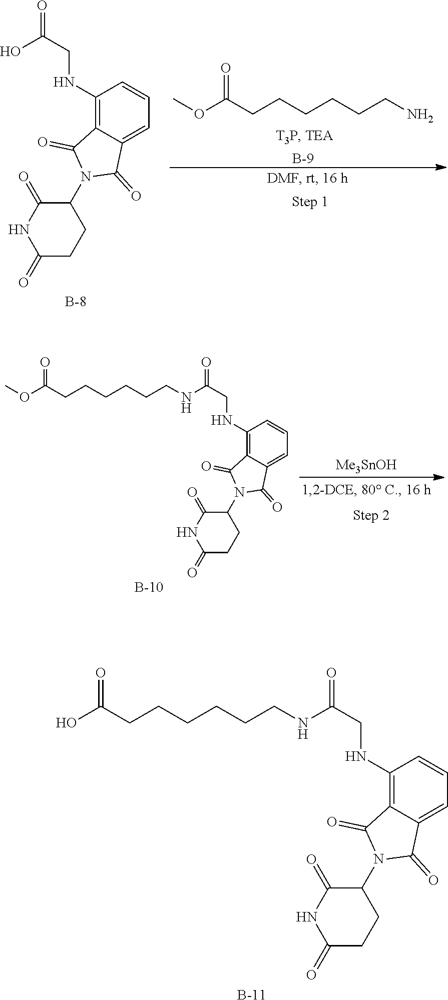

### Step 1: methyl 7-[[2-[[2-(2,6-dioxo-3-piperidyl)-1,3-dioxo-isoindolin-4-yl]amino]acetyl]amino]heptanoate (B-10)

Into a 50 mL round bottom flask containing a well-stirred solution of 2-[[2-(2,6-dioxo-3-piperidyl)-1,3-dioxo-isoindolin-4-yl]amino]acetic acid (B-8, 0.190 g, 573.53 μmol) in anhydrous DMF (0.5 mL) was added triethylamine (174.11 mg, 1.72 mmol, 239.82 μL) and 1-propanephosphonic anhydride solution (50% in Ethyl acetate) (202.00 mg, 630.89 μmol, 0.40 mL). After 10 min, methyl 7-aminoheptanoate (B-9, 91.32 mg, 573.53 μmol) was added. After 16 h, the volatiles were removed under reduced pressure and the residue diluted with water (50 mL) and extracted with Ethyl acetate (2×100 mL). Combined organic layers were dried over sodium sulfate, filtered and the solvent removed under reduced pressure. The residue was purified by flash silica gel (230-400 mesh) column chromatography (30-40% Ethyl acetate in petroleum ether) to afford methyl 7-[[2-[[2-(2,6-dioxo-3-piperidyl)-1,3-dioxo-isoindolin-4-yl]amino]acetyl]amino]heptanoate (B-10, 120 mg, 180.32 μmol, 31% yield) as an off white solid. LCMS (ES+): m/z 473.3 [M+H]⁺.

### Step 2: 7-[[2-[[2-(2,6-dioxo-3-piperidyl)-1,3-dioxo-isoindolin-4-yl]amino]acetyl]amino]heptanoic acid (B-11)

Into a 50 mL single neck round bottom flask containing a well-stirred solution of methyl 7-[[2-[[2-(2,6-dioxo-3-piperidyl)-1,3-dioxo-isoindolin-4-yl]amino]acetyl]amino]heptanoate (B-10, 120.00 mg, 253.97 μmol) in 1,2-dichloroethane (15 mL) was added trimethyltin hydroxide (742.80 mg, 4.11 mmol). The reaction mixture was heated at 80° C. for 16 h. The volatiles were removed under reduced pressure and the residue purified by reverse phase prep HPLC [Purification method: Column: SunFire C18 (19×150 mm) 5 μm, Mobile phase A: 0.1% formic acid in water, Mobile phase B: MeCN; Flow Rate: 15.0 mL/min] to afford 7-[[2-[[2-(2,6-dioxo-3-piperidyl)-1,3-dioxo-isoindolin-4-yl]amino]acetyl]amino]heptanoic acid (B-11, 100 mg, 207.21 μmol, 82% yield, Formic acid salt) as a white solid. LCMS (ES+): m/z 459.1 [M+H]⁺.

### 3-[2-[2-[[2-[[2-(2,6-dioxo-3-piperidyl)-1,3-dioxo-isoindolin-4-yl]oxyacetyl]amino]ethoxy]ethoxy]propanoic acid (B-15)

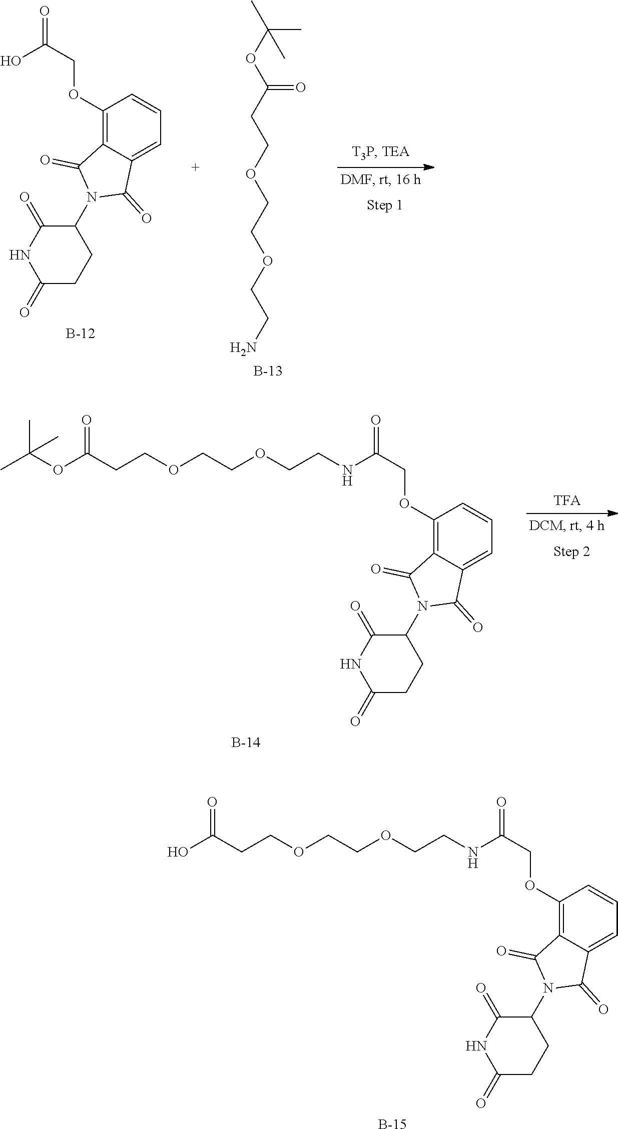

### Step 1: tert-butyl 3-[2-[2-[[2-[[2-(2,6-dioxo-3-piperidyl)-1,3-dioxo-isoindolin-4-yl]oxyacetyl]amino]ethoxy]ethoxy]propanoate (B-14)

tert-butyl 3-[2-[2-[[2-[[2-(2,6-dioxo-3-piperidyl)-1,3-dioxo-isoindolin-4-yl]oxyacetyl]amino]ethoxy]ethoxy]propanoate (B-14, 120 mg, 206.01 μmol, 46% yield) was synthesized from 2-[[2-(2,6-dioxo-3-piperidyl)-1,3-dioxo-isoindolin-4-yl]oxyacetic acid (B-12) and tert-butyl 3-[2-(2-aminoethoxy)ethoxy]propanoate (B-13) in a similar fashion to Compound B-10, except using 2 eq. propanephosphonic anhydride (50% in ethyl acetate). LCMS (ES−): m/z 546.2 [M−H]⁻.

### Step 2: 3-[2-[2-[[2-[[2-(2,6-dioxo-3-piperidyl)-1,3-dioxo-isoindolin-4-yl]oxyacetyl]amino]ethoxy]ethoxy]propanoic acid (B-15)

3-[2-[2-[[2-[[2-(2,6-dioxo-3-piperidyl)-1,3-dioxo-isoindolin-4-yl]oxyacetyl]amino]ethoxy]ethoxy]propanoic acid (B-15, 65 mg, 117.71 mol, 72% yield) was synthesized from tert-butyl 3-[2-[2-[[2-[[2-(2,6-dioxo-3-piperidyl)-1,3-dioxo-isoindolin-4-yl]oxyacetyl]amino]ethoxy]ethoxy]propanoate (B-14) in a similar fashion to Compound A-26, except using 5 eq. TFA. The material was used in the next step without purification. LCMS (ES+): m/z 492.1 [M+H]⁺.

### 7-[[2-[[2-(2,6-dioxo-3-piperidyl)-1,3-dioxo-isoindolin-4-yl]-methyl-amino]acetyl]amino]heptanoic acid (B-19)

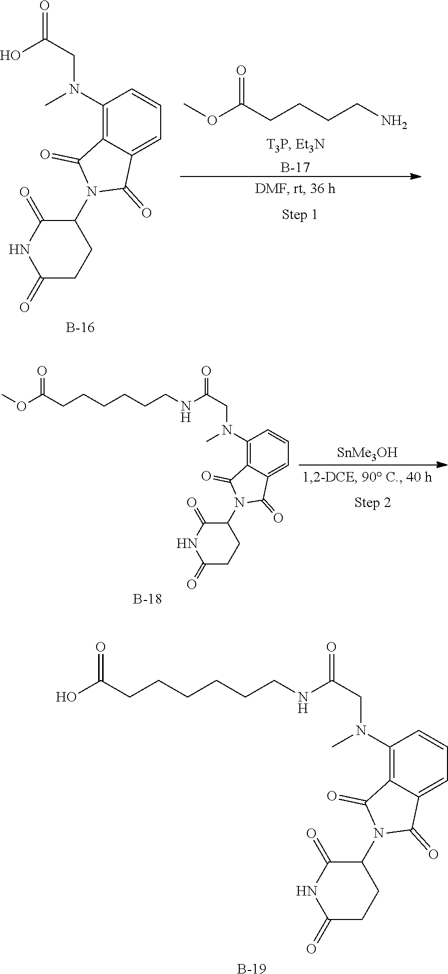

### Step 1: methyl 7-[[2-[[2-(2,6-dioxo-3-piperidyl)-1,3-dioxoisoindolin-4-yl]-methyl-amino]acetyl]amino]heptanoate (B-18)

Into a 50 mL single neck round bottom flask containing a well-stirred solution of 2-[[2-(2,6-dioxo-3-piperidyl)-1,3-dioxo-isoindolin-4-yl]-methyl-amino]acetic acid (B-16, 150 mg, 434.40 μmol) in DMF (5 mL) were added Et₃N (219.78 mg, 2.17 mmol, 302.73 μL) and 1-propanephosphonic anhydride (50% in Ethyl acetate) (207.33 mg, 651.60 μmol, 0.415 mL) at room temperature. After 10 min, 7-amino-heptanoic acid methyl ester hydrochloride (B-17, 102.01 mg, 521.28 μmol) was added. After 16 h, additional 7-aminoheptanoic acid methyl ester hydrochloride (B-17, 425 mg, 2170 μmol) and Et₃N (438 mg, 4.34 mmol) were added in two portions over 2 h and stirring was continued for another 20 h. The reaction mixture was diluted with Ethyl acetate (30 mL) and washed with water (2×10 mL) and brine (10 mL), dried over sodium sulfate, filtered and solvent removed to obtain methyl 7-[[2-[[2-(2,6-dioxo-3-piperidyl)-1,3-dioxo-isoindolin-4-yl]-methyl-amino]acetyl]amino]heptanoate (B-18, 250 mg, 272.34 μmol, 63% yield) as a yellow solid. The material was used in the next step without further purification. LCMS (ES−): m/z 485.2 [M−H]⁻.

### Step 2: 7-[[2-[[2-(2,6-dioxo-3-piperidyl)-1,3-dioxo-isoindolin-4-yl]-methyl-amino]acetyl]amino]heptanoic acid (B-19)

7-[2-[[2-(2,6-dioxo-3-piperidyl)-1,3-dioxo-isoindolin-4-yl]-methyl-amino]acetyl]amino]heptanoic acid (B-19, 32 mg, 50.12 μmol, 12% yield) was synthesized from methyl 7-[[2-[[2-(2,6-dioxo-3-piperidyl)-1,3-dioxo-isoindolin-4-yl]-methyl-amino]acetyl]amino]heptanoate (B-18) in a similar fashion to Compound B-11, except using 20 eq. trimethyltin hydroxide added in two portions over 16 h. Upon completion, the reaction mixture was concentrated to dryness before Ethyl acetate (50 mL) and 0.5 N HCl solution (20 mL) were added and stirred for 30 min. The layers were separated, the aqueous layer was extracted with Ethyl acetate (30 mL) and 15% MeOH in DCM. The combined organic layer was dried over sodium sulfate, filtered and concentrated. The residue was purified by reverse phase preparative HPLC [Column: SunFire C18 (19×150 mm) 5 μm, Mobile phase: 0.1% TFA in water and MeCN]. LCMS (ES+): m/z 473.3 [M+H]⁺.

### 3-[2-[2-[2-[[2-(2,6-dioxo-3-piperidyl)-1,3-dioxo-isoindolin-4-yl]oxyethoxy]ethoxy]ethoxy]propanoic acid (B-26)

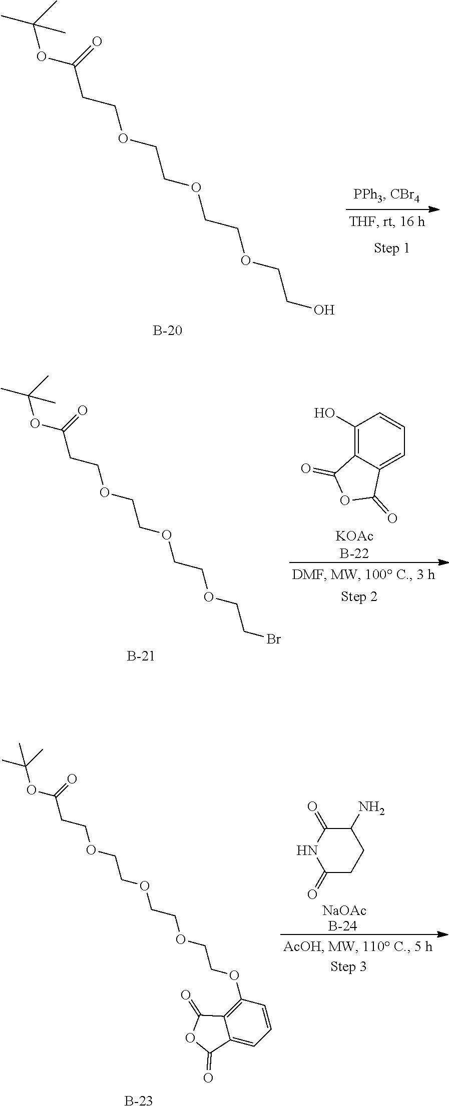

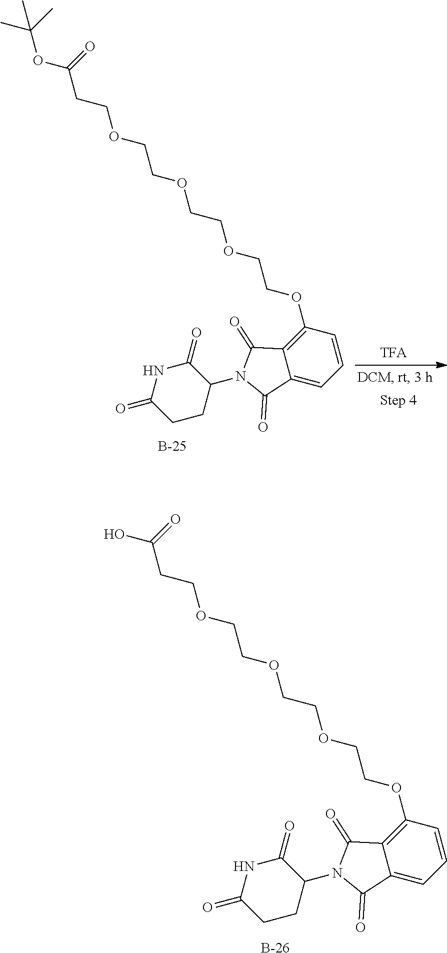

### Step 1: tert-butyl 3-[2-[2-(2-bromoethoxy)ethoxy]ethoxy]propanoate (B-21)

Into a 50 mL two neck round bottom flask containing a well-stirred solution of tert-butyl 3-[2-[2-(2-hydroxyethoxy)ethoxy]ethoxy]propanoate (B-20, 1.0 g, 3.59 mmol) in THF (15 mL) were added triphenyl phosphine (1.41 g, 5.39 mmol) and carbon tetrabromide (2.38 g, 7.19 mmol, 696.75 μL) at room temperature. After 16 h, the reaction mixture was diluted with ice water (75 mL) and extracted with ethyl acetate (3×75 mL). The combined organic layer was dried over anhydrous sodium sulfate, filtered and concentrated under reduced pressure. The residue was purified by flash silica gel (100-200 mesh) column chromatography (50% Ethyl acetate in petroleum ether) to afford tert-butyl 3-[2-[2-(2-bromoethoxy)ethoxy]ethoxy]propanoate (B-21, 1.2 g, 3.49 mmol, 97% yield) as colourless liquid. LCMS (ES+): m/z 285.0 [M−tBu+H]⁺.

### Step 2: tert-butyl 3-[2-[2-[2-(1,3-dioxoisobenzofuran-4-yl)oxyethoxy]ethoxy]ethoxy]propanoate (B-23)

Into a 20 mL microwave vial containing a well-stirred solution of 4-hydroxyisobenzofuran-1,3-dione (B-22, 0.5 g, 3.05 mmol) in DMF (10 mL) was added potassium acetate (448.51 mg, 4.57 mmol, 285.67 μL) and stirred for 10 min. Then, tert-butyl 3-[2-[2-(2-bromoethoxy)ethoxy]ethoxy]propanoate (B-21, 1.25 g, 3.66 mmol) was added. The tube was sealed and irradiated under microwave conditions at 100° C. for 3 h. The reaction mixture was diluted with water and extracted with ethyl acetate (3×30 mL). The combined organic layers were washed with ice water (2×20 mL) and brine (20 mL), dried over anhydrous sodium sulfate, filtered and concentrated under reduced pressure to afford tert-butyl 3-[2-[2-[2-(1,3-dioxoisobenzofuran-4-yl)oxyethoxy]ethoxy]ethoxy]propanoate (B-23, 1.5 g, 397.58 μmol, 13% yield) as thick red liquid which was used in the next step without further purification. LCMS (ES+): m/z 369.2 [M−(Bu+H]⁺.

### Step 3: tert-butyl3-[2-[2-[2-[[2-(2,6-dioxo-3-piperidyl)-1,3-dioxo-isoindolin-4-yl]oxyethoxy]ethoxy]ethoxy]propanoate (B-25)

Into a 100 mL microwave vial containing a well-stirred solution of tert-butyl 3-[2-[2-[2-(1,3-dioxoisobenzofuran-4-yl)oxyethoxy]ethoxy]ethoxy]propanoate (B-23, 1.5 g, 3.53 mmol) in acetic acid (10 mL) was added sodium acetate (579.80 mg, 7.07 mmol, 378.95 μL) and the suspension was stirred for 10 min. Then, 3-aminopiperidine-2,6-dione hydrochloride (B-24, 581.67 mg, 3.53 mmol) was added. The tube was sealed and subjected to microwave irradiation at 110° C. for 5 h. The reaction mixture was diluted with water (75 mL) and extracted with Ethyl acetate (3×75 mL). The combined organic layer was washed with ice water (2×50 mL) and brine (50 mL), dried over sodium sulfate, filtered and concentrated under reduced pressure. The residue was purified by reverse phase prep HPLC [Purification method: Column: SunFire C18 (19×150 mm) 5 μm; Mobile phase A: 0.1% TFA in water and Mobile phase B: MeCN] to afford tert-butyl 3-[2-[2-[2-[[2-(2,6-dioxo-3-piperidyl)-1,3-dioxo-isoindolin-4-yl]oxyethoxy]ethoxy]ethoxy]propanoate (B-25, 320 mg, 573.07 μmol, 16% yield) as a light yellow solid. LCMS (ES+): m/z 479.0 [M−tBu+H]⁺.

### Step 4: 3-[2-[2-[2-[[2-(2,6-dioxo-3-piperidyl)-1,3-dioxo-isoindolin-4-yl]oxyethoxy]ethoxy]ethoxy]propanoic acid (B-26)

3-[2-[2-[2-[[2-(2,6-dioxo-3-piperidyl)-1,3-dioxo-isoindolin-4-yl]oxyethoxy]ethoxy]ethoxy]propanoic acid (B-26, 300 mg, 508.27 μmol, 85% yield) was synthesized from tert-butyl 3-[2-[2-[2-[[2-(2,6-dioxo-3-piperidyl)-1,3-dioxo-isoindolin-4-yl]oxyethoxy]ethoxy]ethoxy]propanoate (B-25) in a similar fashion to Compound A-26, except using 6.5 eq. TFA. The material was used in the next step without further purification. LCMS (ES+): m/z 479.1 [M+H]⁺.

### 3-[2-[2-[[2-[[2-(2,6-dioxo-3-piperidyl)-1-oxo-isoindolin-4-yl]oxyacetyl]amino]ethoxy]ethoxy]propanoic acid (B-29)

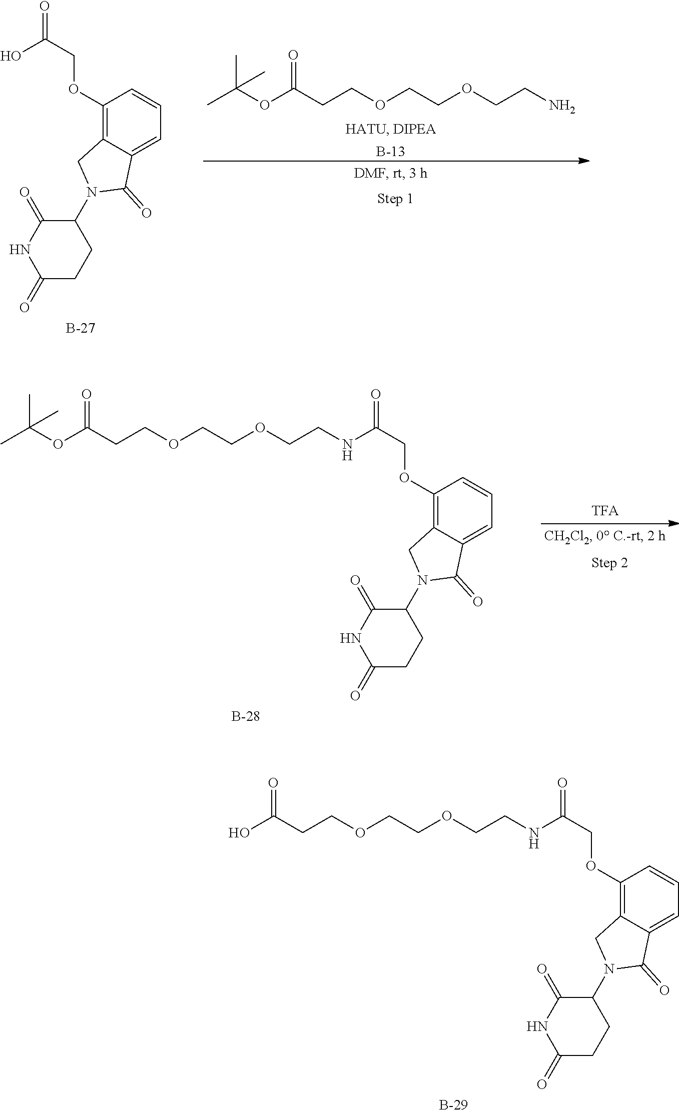

### Step 1: 3-[2-[2-[[2-[[2-(2,6-dioxo-3-piperidyl)-1-oxo-isoindolin-4-yl]oxyacetyl]-tert-butyl amino]ethoxy]ethoxy]propanoate (B-28)

Into a 50 mL single neck round bottom flask containing a well-stirred solution of 2-[[2-(2,6-dioxo-3-piperidyl)-1-oxo-isoindolin-4-yl]oxyacetic acid (B-27, 125 mg, 392.73 μmol) and tert-butyl 3-[2-(2-aminoethoxy)ethoxy]propanoate (B-13, 91.63 mg, 392.73 μmol) in DMF (2 mL) were added DIPEA (152.27 mg, 1.18 mmol, 205.22 μL) and HATU (225.18 mg, 589.10 μmol). After 3 h, Ethyl acetate (30 mL) was added to the reaction mixture and washed with water (2×10 mL) and brine (10 mL), dried over anhydrous sodium sulfate, filtered and concentrated. The residue was purified by flash silica gel (230-400 mesh) column chromatography (85% Ethyl acetate in pet. ether) to afford tert-butyl 3-[2-[2-[[2-[[2-(2,6-dioxo-3-piperidyl)-1-oxo-isoindolin-4-yl]oxyacetyl]amino]ethoxy]ethoxy]propanoate (B-28, 140 mg, 220.85 μmol, 56% yield) as an off-white solid. LCMS (ES−): m/z 532.2 [M−H]⁻.

### Step 2: 3-[2-[2-[[2-[[2-(2,6-dioxo-3-piperidyl)-1-oxo-isoindolin-4-yl]oxyacetyl]amino]ethoxy]ethoxy]propanoic acid (B-29)

3-[2-[2-[[2-[[2-(2,6-dioxo-3-piperidyl)-1-oxo-isoindolin-4-yl]oxyacetyl]amino]ethoxy]ethoxy]propanoic acid (B-29, 70 mg, 143.53 μmol, 48% yield) was synthesized from tert-butyl 3-[2-[2-[[2-[[2-(2,6-dioxo-3-piperidyl)-1-oxo-isoindolin-4-yl]oxyacetyl]amino]ethoxy]ethoxy]propanoate (B-28) in a similar fashion to Compound A-26, except the material was purified by reverse phase preparative HPLC [Column: SunFire C18 (19×150 mm) 5 μm, Mobile phase: 0.1% TFA in water and MeCN]. LCMS (ES+): m/z 478.2 [M+H]⁺.

### 7-[[2-[[2-(2,6-dioxo-3-piperidyl)-1-oxo-isoindolin-4-yl]oxyacetyl]amino]heptanoic acid (B-31)

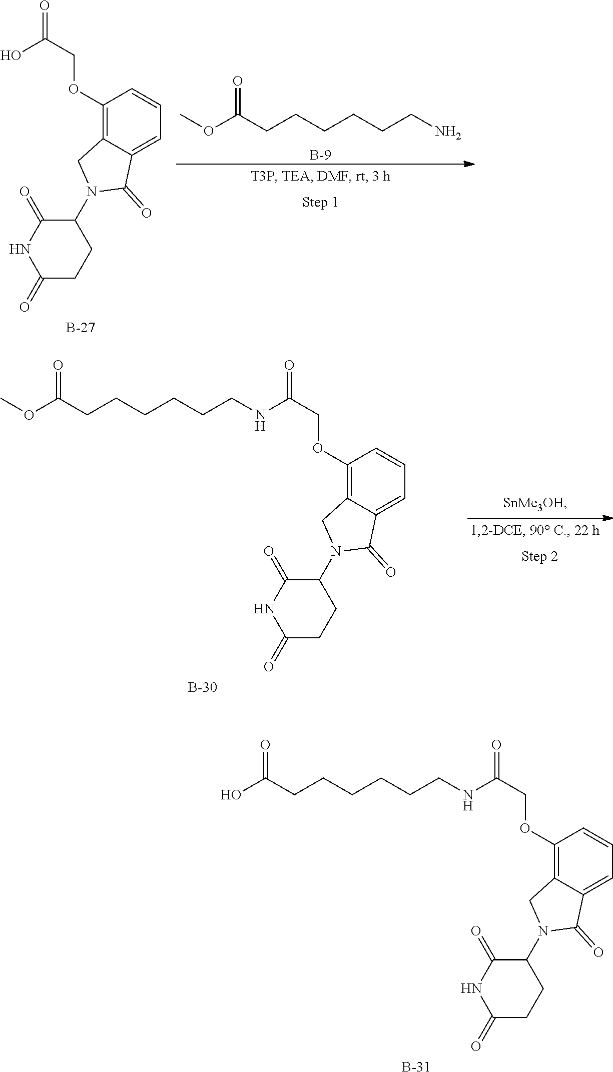

### Step 1: methyl 7-[[2-[[2-(2,6-dioxo-3-piperidyl)-1-oxo-isoindolin-4-yl]oxyacetyl]amino]heptanoate (B-30)

methyl 7-[[2-[[2-(2,6-dioxo-3-piperidyl)-1-oxo-isoindolin-4-yl]oxyacetyl]amino]heptanoate (B-30, 280 mg, 539.11 μmol, 66% yield) was synthesized from 2-[[2-(2,6-dioxo-3-piperidyl)-1-oxo-isoindolin-4-yl]oxyacetic acid (B-27) and methyl 7-aminoheptanoate hydrochloride (B-9) in a similar fashion to Compound B-10, except using 2 eq. triethylamine and 2 eq. 1-propanephosphonic anhydride solution (50% in Ethyl acetate). UPLC-MS (ES+): m/z 460.5 [M+H]⁺.

### Step 2: 7-[[2-[[2-(2,6-dioxo-3-piperidyl)-1-oxo-isoindolin-4-yl]oxyacetyl]amino]heptanoic acid (B-31)

7-[[2-[[2-(2,6-dioxo-3-piperidyl)-1-oxo-isoindolin-4-yl]oxyacetyl]amino]heptanoic acid (B-31, 52 mg, 115.56 μmol, 19% yield) was synthesized from methyl 7-[[2-[[2-(2,6-dioxo-3-piperidyl)-1-oxo-isoindolin-4-yl]oxyacetyl]amino]heptanoate (B-30) in a similar fashion to Compound B-11, except upon completion, the solvent was removed under reduced pressure and the residue was partitioned between Ethyl acetate and aq. 0.5 N HCl solution and stirred for 30 min. The layers were separated, the aqueous layer was extracted with ethyl acetate (2×) and the combined organic layer was washed with 0.5 N HCl and brine, dried over sodium sulfate, concentrated and purified by reverse phase prep HPLC [Purification method: Column: SunFire C18 (19×150 mm) 5 μm, Mobile phase: 0.1% TFA in water and MeCN]. LCMS (ES+): m/z 446.2 [M+H]⁺.

### 1-[2-[[2-(2,6-dioxo-3-piperidyl)-1,3-dioxo-isoindolin-4-yl]amino]acetyl]piperidine-4-carboxylic acid (B-33)

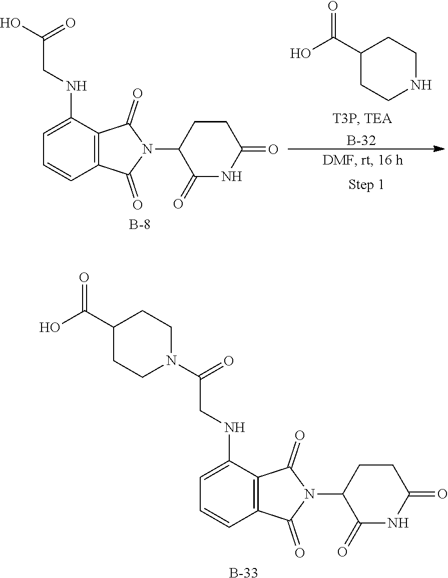

### Step 1: 1-[2-[[2-(2,6-dioxo-3-piperidyl)-1,3-dioxo-isoindolin-4-yl]amino]acetyl]piperidine-4-carboxylic acid (B-33)

1-[2-[[2-(2,6-dioxo-3-piperidyl)-1,3-dioxo-isoindolin-4-yl]amino]acetyl]piperidine-4-carboxylic acid (B-33, 55 mg, 121.83 μmol, 45% yield, TFA salt) was synthesized from 2-[[2-(2,6-dioxo-3-piperidyl)-1,3-dioxo-isoindolin-4-yl]amino]acetic acid (B-8) and piperidine-4-carboxylic acid (B-32) in a similar fashion to Compound B-10, except using 5 eq. triethylamine and 2 eq. 1-propanephosphonic anhydride solution (50% in ethyl acetate). Upon completion, the volatiles were removed under reduced pressure and the residue was purified by reverse phase prep HPLC [Purification method: Column: SunFire-C18 100 mm, Mobile phase A: 0.1% TFA in Water; Mobile phase B: MeCN, Flow Rate: 15.0 mL/min]. LCMS (ES+): m/z 443.1 [M+H]⁺.

### 7-[[2-[[2-(2,6-dioxo-3-piperidyl)-1-oxo-isoindolin-4-yl]amino]acetyl]amino]heptanoic acid (B-36)

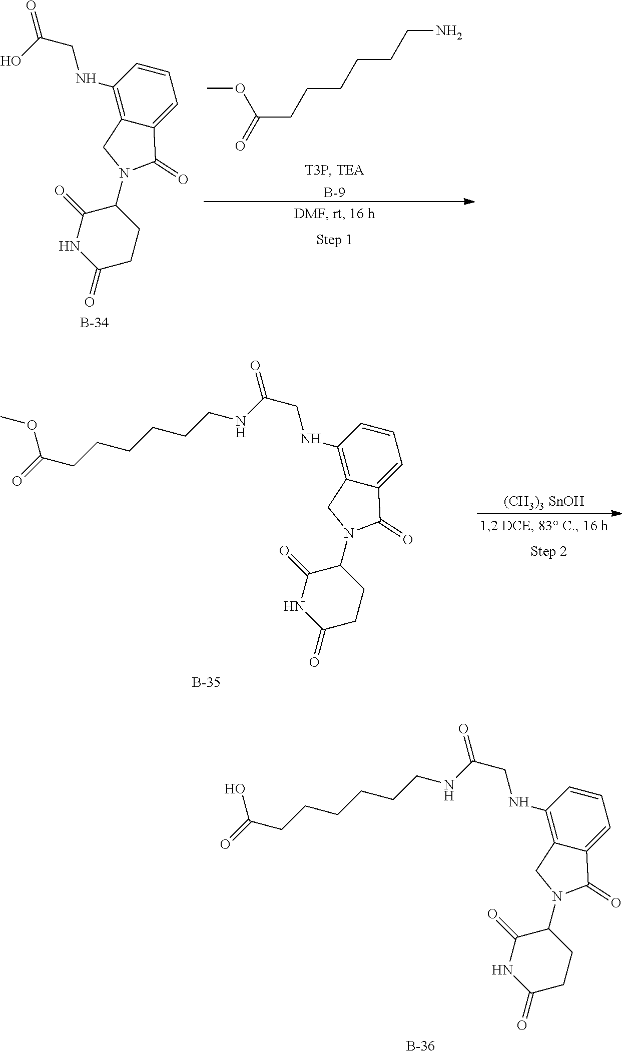

### Step 1: methyl 7-[[2-[[2-(2,6-dioxo-3-piperidyl)-1-oxo-isoindolin-4-yl]amino]acetyl]amino]heptanoate (B-35)

methyl 7-[[2-[[2-(2,6-dioxo-3-piperidyl)-1-oxo-isoindolin-4-yl]amino]acetyl]amino]heptanoate (B-35, 125 mg, 150.03 μmol, 10% yield) was synthesized from 2-[[2-(2,6-dioxo-3-piperidyl)-1-oxo-isoindolin-4-yl]amino]acetic acid (B-34) and methyl 7-aminoheptanoate hydrochloride (B-9) in a similar fashion to Compound B-10, except using 2 eq. 1-propanephosphonic anhydride solution (50% in Ethyl acetate). LCMS (ES+): m/z 459.1 [M+H]⁺.

### Step 2: 7-[[2-[[2-(2,6-dioxo-3-piperidyl)-1-oxo-isoindolin-4-yl]amino]acetyl]amino]heptanoic acid (B-36)

7-[[2-[[2-(2,6-dioxo-3-piperidyl)-1-oxo-isoindolin-4-yl]amino]acetyl]amino]heptanoic acid (B-36, 80 mg, 172.35 μmol, 32% yield) was synthesized from methyl 7-[[2-[[2-(2,6-dioxo-3-piperidyl)-1-oxo-isoindolin-4-yl]amino]acetyl]amino]heptanoate (B-35) in a similar fashion to Compound B-11, except using 5 eq. trimethyltin hydroxide. LCMS (ES+): m/z 445.1 [M+H]⁺.

3-(2-(2-(3-(2-(2,6-dioxopiperidin-3-yl)-1-oxoisoindolin-5-yl)propoxy)ethoxy)ethoxy)propanoic acid (B-40)

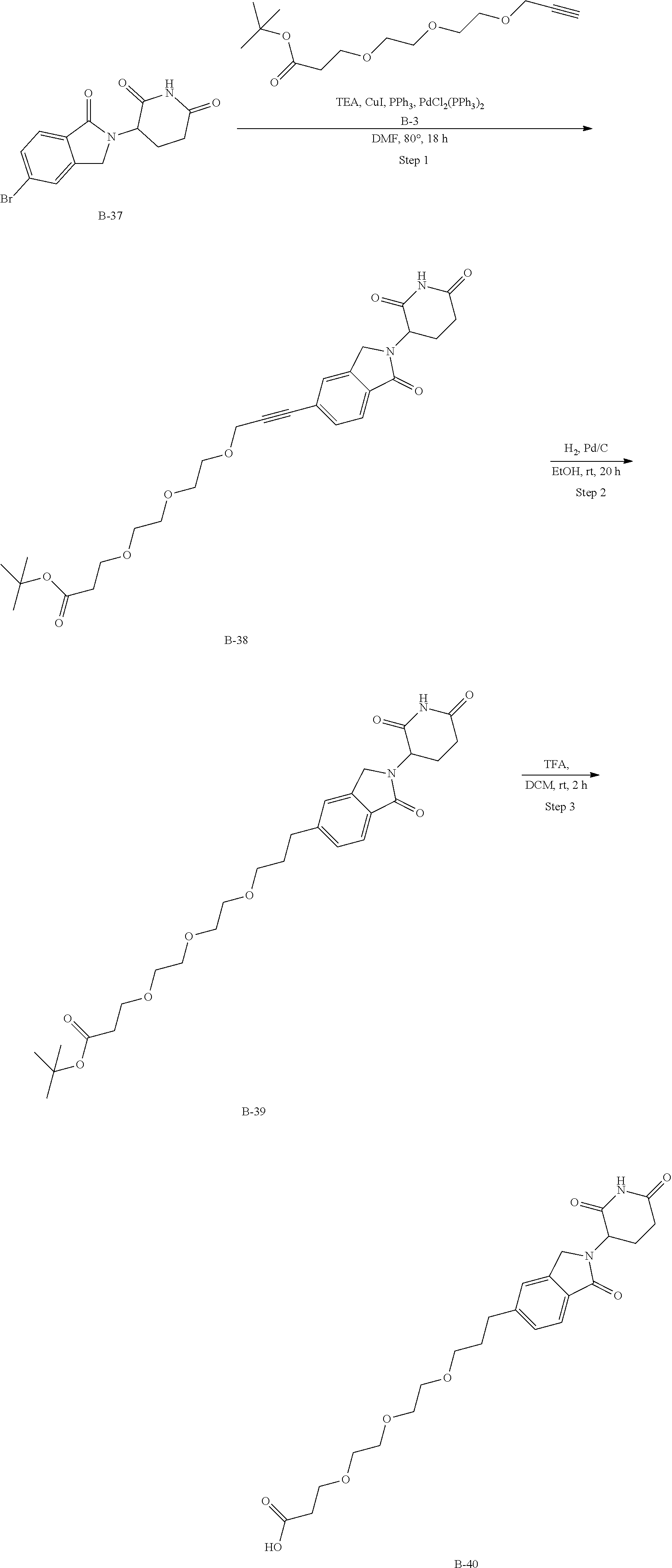

### Step 1: tert-butyl 3-(2-(2-((3-(2-(2,6-dioxopiperidin-3-yl)-1-oxoisoindolin-5-yl) prop-2-yn-1-yl)oxy)ethoxy)ethoxy)propanoate (B-38)

tert-butyl 3-(2-(2-((3-(2-(2,6-dioxopiperidin-3-yl)-1-oxoisoindolin-5-yl) prop-2-yn-1-yl)oxy)ethoxy)ethoxy)propanoate (B-38, 100 mg, 153.53 μmol, 12% yield) was synthesized from tert-butyl 3-[2-(2-prop-2-ynoxyethoxy)ethoxy]propanoate (B-3) and 3-(5-bromo-1-oxo-isoindolin-2-yl)piperidine-2,6-dione (B-37) in a similar fashion to Compound B-5, except using 0.1 eq. Cul. LCMS (ES+): m/z 459.1 [M−tBu+H]⁺.

### Step 2: tert-butyl 3-(2-(2-(3-(2-(2,6-dioxopiperidin-3-yl)-1-oxoisoindolin-5-yl)propoxy) ethoxy)ethoxy)propanoate (B-39)

tert-butyl 3-(2-(2-(3-(2-(2,6-dioxopiperidin-3-yl)-1-oxoisoindolin-5-yl)propoxy)ethoxy)ethoxy)propanoate (B-39, 60 mg, 65.9 μmol, 27% yield) was synthesized from tert-butyl 3-(2-(2-((3-(2-(2,6-dioxopiperidin-3-yl)-1-oxoisoindolin-5-yl) prop-2-yn-1-yl)oxy)ethoxy)ethoxy)propanoate (B-38) in a similar fashion to Compound B-6, except using 0.1 eq. palladium (10% on carbon). The material was used in the next step without further purification. LCMS (ES+): m/z 463.1 [M−tBu+H]⁺.

### Step 3-(2-(2-(3-(2-(2,6-dioxopiperidin-3-yl)-1-oxoisoindolin-5-yl)propoxy)ethoxy)ethoxy)propanoic acid (B-40)

3-(2-(2-(3-(2-(2,6-dioxopiperidin-3-yl)-1-oxoisoindolin-5-yl)propoxy)ethoxy)ethoxy)propanoic acid (B-40, 50 mg, 104.87 μmol, 47% yield) was synthesized from tert-butyl 3-(2-(2-(3-(2-(2,6-dioxopiperidin-3-yl)-1-oxoisoindolin-5-yl)propoxy)ethoxy)ethoxy)propanoate (B-39) in a similar fashion to Compound A-26, except using 15 eq. TFA. The material was purified by reverse phase preparative HPLC [Purification method: Column: SunFire prep OBD (19×50 mm), 5 μm; Mobile phase A: 0.1% TFA in water and Mobile phase B: MeCN]. LCMS (ES+): m/z 462.9 [M+H]⁺.

### 3-[2-[3-[[2-(2,6-dioxo-3-piperidyl)-1,3-dioxo-isoindolin-4-yl]propoxy]ethoxy]propanoic acid (B-45)

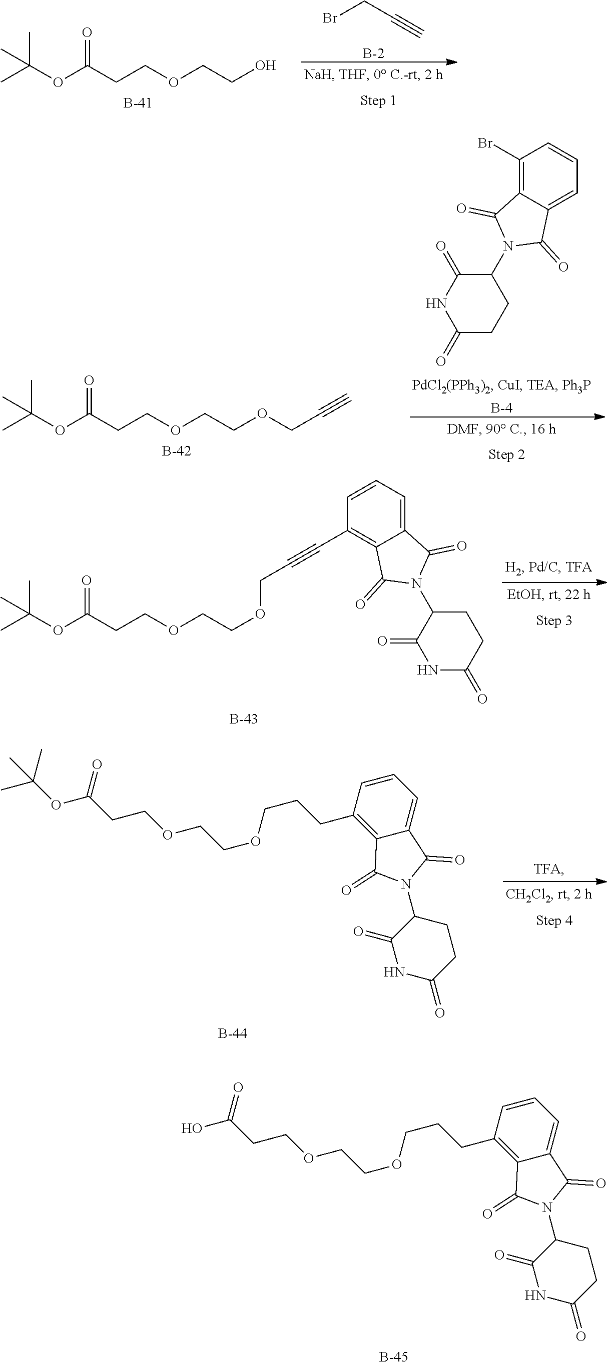

### Step 1: tert-butyl 3-(2-prop-2-ynoxyethoxy)propanoate (B-42)

tert-butyl 3-(2-prop-2-ynoxyethoxy)propanoate (B-42, 500 mg, 2.17 mmol, 26% yield) was synthesized from tert-butyl 3-(2-hydroxyethoxy)propanoate (B-41) and 3-bromoprop-1-yne (80% in toluene) in a similar fashion to Compound B-3.

### Step 2: tert-butyl 3-[2-[3-[[2-(2,6-dioxo-3-piperidyl)-1,3-dioxo-isoindolin-4-yl]prop-2-ynoxy]ethoxy]propanoate (B-43)

tert-butyl 3-[2-[3-[[2-(2,6-dioxo-3-piperidyl)-1,3-dioxo-isoindolin-4-yl]prop-2-ynoxy]ethoxy]propanoate (B-43, 180 mg, 158.93 μmol, 18% yield) was synthesized from 4-bromo-2-(2,6-dioxo-3-piperidyl)isoindoline-1,3-dione (B-4) and tert-butyl 3-(2-prop-2-ynoxyethoxy)propanoate (B-42) in a similar fashion to Compound B-5, except using 1.1 eq. B-4 and 0.2 eq. Cul. LCMS (ES−): m/z 483.3 [M−H]⁺.

### Step 3: tert-butyl Mar. 12, 2013-[[2-(2,6-dioxo-3-piperidyl)-1,3-dioxo-isoindolin-4-yl]propoxy]ethoxy]propanoate (B-44)

tert-butyl 3-[2-[3-[[2-(2,6-dioxo-3-piperidyl)-1,3-dioxo-isoindolin-4-yl]propoxy]ethoxy]propanoate (B-44, 150 mg, 128.96 μmol, 35% yield) was synthesized from tert-butyl 3-[2-[3-[[2-(2,6-dioxo-3-piperidyl)-1,3-dioxo-isoindolin-4-yl]prop-2-ynoxy]ethoxy]propanoate (B-43) in a similar fashion to Compound B-6, except adding 0.2 eq. TFA. The material was used in the next step without further purification. LCMS (ES−): m/z 487 [M−H]⁻.

### Step 4: 3-[2-[3-[[2-(2,6-dioxo-3-piperidyl)-1,3-dioxo-isoindolin-4-yl]propoxy]ethoxy]propanoic acid (B-45)

3-[2-[3-[[2-(2,6-dioxo-3-piperidyl)-1,3-dioxo-isoindolin-4-yl]propoxy]ethoxy]propanoic acid (B-45, 45 mg, 99.90 μmol, 33% yield) was synthesized from tert-butyl 3-[2-[3-[[2-(2,6-dioxo-3-piperidyl)-1,3-dioxo-isoindolin-4-yl]propoxy]ethoxy]propanoate (B-44) in a similar fashion to Compound A-26, except using 42 eq. TFA. The material was purified by reverse-phase preparative HPLC [Column: SunFire C18 (19×150 mm) 5 μm, mobile phase: 0.1% TFA in water and MeCN]. LCMS (ES+): m/z 433.2 [M+H]⁺.

### 6-[[2-[[2-(2,6-dioxo-3-piperidyl)-1-oxo-isoindolin-4-yl]amino]acetyl]amino]hexanoic acid (B-48)

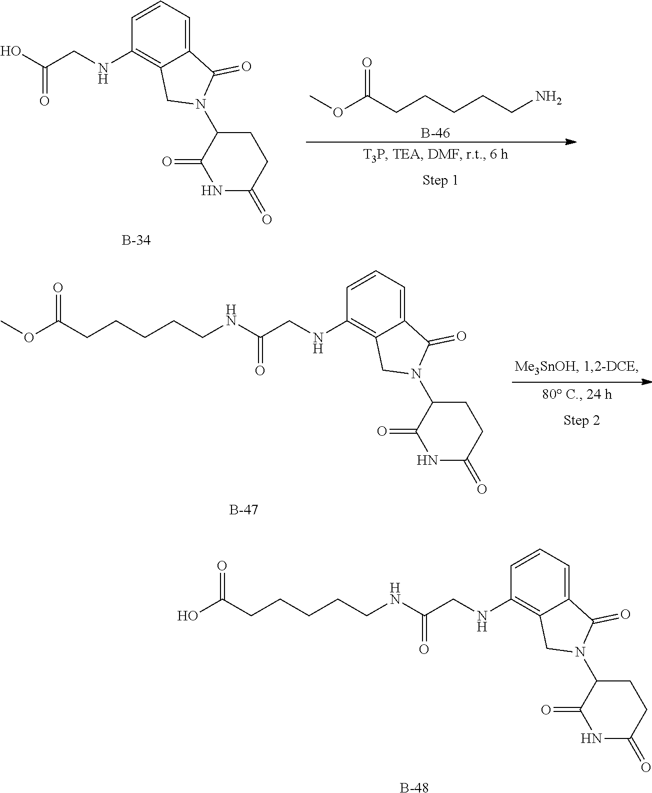

### Step 1: methyl 6-[[2-[[2-(2,6-dioxo-3-piperidyl)-1-oxo-isoindolin-4-yl]amino]acetyl]amino]hexanoate (B-47)

methyl 6-[[2-[[2-(2,6-dioxo-3-piperidyl)-1-oxo-isoindolin-4-yl]amino]acetyl]amino]hexanoate (B-47, 0.23 g, 320.82 μmol, 34% yield) was synthesized from 2-[[2-(2,6-dioxo-3-piperidyl)-1-oxo-isoindolin-4-yl]amino]acetic acid (B-34) and methyl 6-aminohexanoate hydrochloride (B-46) in a similar fashion to Compound B-10, except using 2 eq. B-46 and 1 eq. 1-propanephosphonic anhydride solution (50 wt. % in ethyl acetate). LCMS (ES+): m/z 444.9 [M+H]⁺.

### Step 2: 6-[[2-[[2-(2,6-dioxo-3-piperidyl)-1-oxo-isoindolin-4-yl]amino]acetyl]amino]hexanoic acid (B-48)

6-[[2-[[2-(2,6-dioxo-3-piperidyl)-1-oxo-isoindolin-4-yl]amino]acetyl]amino]hexanoic acid (B-48, 45 mg, 82.72 μmol, 16% yield, TFA salt) was synthesized from methyl 6-[2-[[2-(2,6-dioxo-3-piperidyl)-1-oxo-isoindolin-4-yl]amino]acetyl]amino]hexanoate (B-47) in a similar fashion to Compound B-11, except using 6 eq. trimethyltin hydroxide. LCMS (ES+): m/z 430.9 [M+H]⁺.

### 5-[[2-[[2-(2,6-dioxo-3-piperidyl)-1-oxo-isoindolin-4-yl]amino]acetyl]amino]pentanoic acid (B-51)

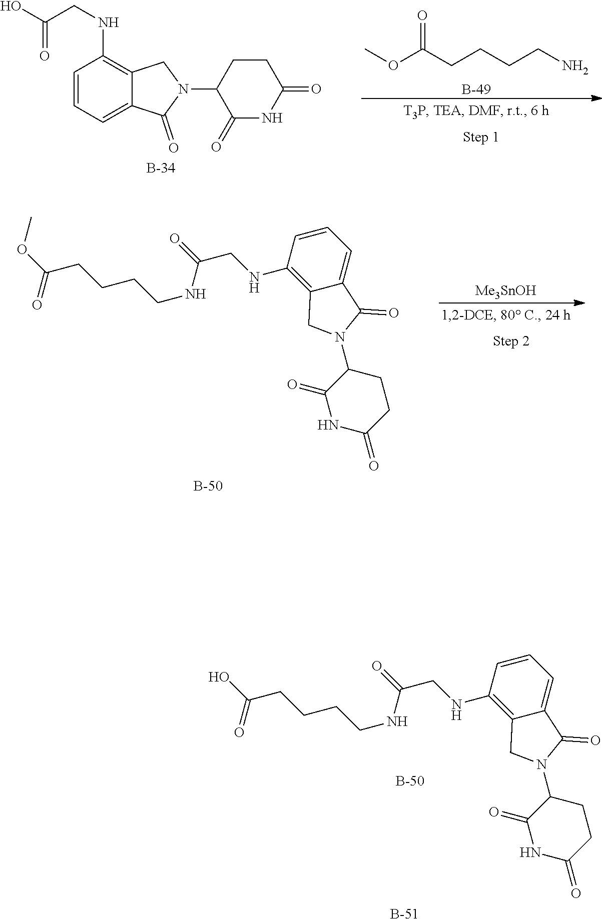

### Step 1: methyl 5-[[2-[[2-(2,6-dioxo-3-piperidyl)-1-oxo-isoindolin-4-yl]amino]acetyl]amino]pentanoate (B-50)

methyl 5-[2-[[2-(2,6-dioxo-3-piperidyl)-1-oxo-isoindolin-4-yl]amino]acetyl]amino]pentanoate (B-50, 230 mg, 309.91 μmol, 33% yield) was synthesized from 2-[[2-(2,6-dioxo-3-piperidyl)-1-oxo-isoindolin-4-yl]amino]acetic acid (B-34) and methyl 5-aminopentanoate hydrochloride (B-49) in a similar fashion to Compound B-10, except using 2 eq. B-49 and 1.5 eq. propylphosphonic acid anhydride solution (50 wt. % in ethyl acetate), LCMS (ES+): m/z 431.1 [M+H]⁺.

### Step 2: 5-[[2-[[2-(2,6-dioxo-3-piperidyl)-1-oxo-isoindolin-4-yl]amino]acetyl]amino]pentanoic acid (B-51)

5-[[2-[[2-(2,6-dioxo-3-piperidyl)-1-oxo-isoindolin-4-yl]amino]acetyl]amino]pentanoic acid (B-51, 70 mg, 132.07 μmol, 25% yield, TFA salt) was synthesized from methyl 5-[[2-[[2-(2,6-dioxo-3-piperidyl)-1-oxo-isoindolin-4-yl]amino]acetyl]amino]pentanoate (B-50) in a similar fashion to Compound B-11, except using 10 eq. trimethyltin hydroxide. LCMS (ES+): m/z 417.2 [M+H]⁺.

### 2-[[2-(2,6-dioxo-3-piperidyl)-1,3-dioxo-isoindolin-5-yl]amino]acetic acid (B-54)

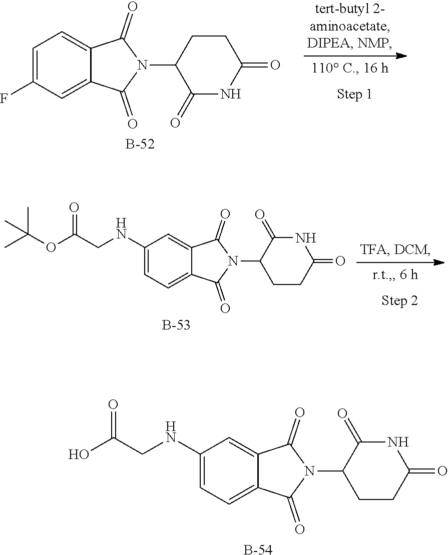

### Step 1: tert-butyl 2-[[2-(2,6-dioxo-3-piperidyl)-1,3-dioxo-isoindolin-5-yl]amino]acetate (B-53)

Into a 250 mL sealed tube containing a well-stirred solution of 2-(2,6-dioxo-3-piperidyl)-5-fluoro-isoindoline-1,3-dione (B-52, 8 g, 28.96 mmol) in anhydrous NMP (50 mL) were added tert-butyl 2-aminoacetate (7.28 g, 43.44 mmol) and N,N-diisopropylethylamine (18.72 g, 144.81 mmol, 25.22 mL) at room temperature under nitrogen atmosphere. The reaction mixture was stirred at 110° C. for 16 h. The solvent was removed under reduced pressure and the residue was purified by flash silica gel (230-400 mesh) column chromatography (30% Ethyl acetate in petroleum ether) to afford tert-butyl 2-[[2-(2,6-dioxo-3-piperidyl)-1,3-dioxo-isoindolin-5-yl]amino]acetate (B-53, 2.1 g, 3.98 mmol, 14% yield) as a yellow solid. LCMS (ES+): m/z 332.0 [M−tBu+H]⁺.

### Step 2: 2-[[2-(2,6-dioxo-3-piperidyl)-1,3-dioxo-isoindolin-S-yl]amino]acetic acid (B-54)

2-[[2-(2,6-dioxo-3-piperidyl)-1,3-dioxo-isoindolin-5-yl]amino]acetic acid (B-54, 1.5 g, 3.14 mmol, 58% yield, TFA salt) was synthesized from tert-butyl 2-[[2-(2,6-dioxo-3-piperidyl)-1,3-dioxo-isoindolin-5-yl]amino]acetate (B-53) in a similar fashion to Compound A-26, except using 10 eq. TFA. The material was azeotroped with toluene and used in the next step without further purification. LCMS (ES+): m/z 332.0 [M+H]⁺.

### 3-[2-[[2-[[2-(2,6-dioxo-3-piperidyl)-1,3-dioxo-isoindolin-5-yl]amino]acetyl]amino]ethoxy]propanoic acid (B-57)

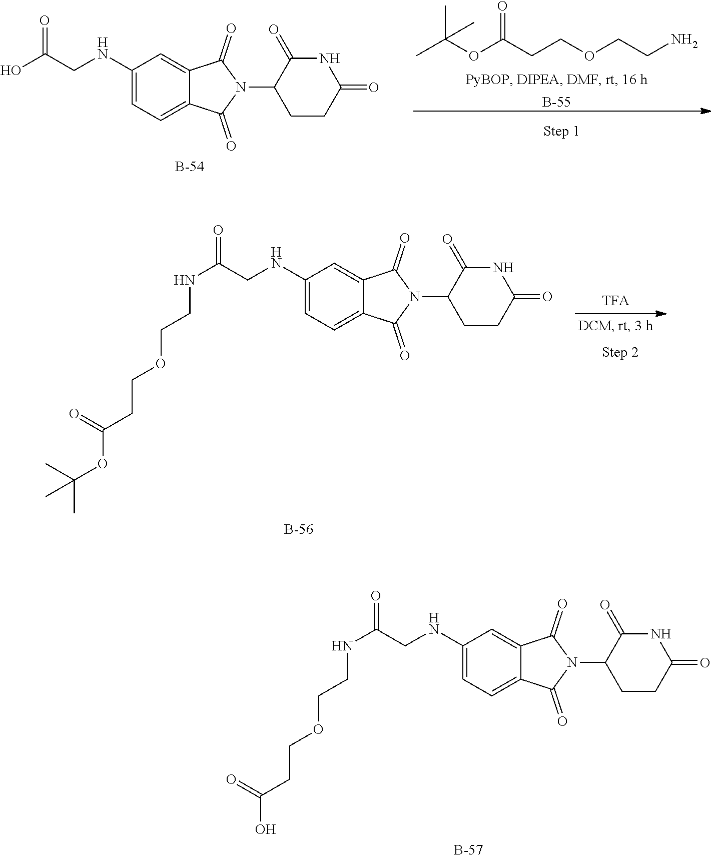

### Step 1: tert-butyl 3-[2-[[2-[[2-(2,6-dioxo-3-piperidyl)-1,3-dioxo-isoindolin-5-yl]amino]acetyl]amino]ethoxy]propanoate (B-56)

Into a 25 mL single neck round bottom flask containing a well-stirred solution of 2-[[2-(2,6-dioxo-3-piperidyl)-1,3-dioxo-isoindolin-5-yl]amino]acetic acid (B-54, 100 mg, 301.86 μmol) in anhydrous DMF were added tert-butyl 3-(2-aminoethoxy)propanoate (B-55, 68.55 mg, 362.23 μmol), DIPEA (117.04 mg, 905.58 μmol, 157.74 μL) and PyBOP (172.79 mg, 332.05 μmol) at room temperature. The reaction mixture was stirred at room temperature for 16 h. The reaction mixture was diluted with water (5 mL) and extracted with 10% MeOH in DCM (20 mL). The organic layer was washed with brine (5 mL), dried over sodium sulfate, filtered and concentrated under reduced pressure. The residue was purified by column chromatography (4-5% MeOH in DCM) to afford tert-butyl 3-[2-[[2-[[2-(2,6-dioxo-3-piperidyl)-1,3-dioxo-isoindolin-5-yl]amino]acetyl]amino]ethoxy]propanoate (B-56, 150 mg, 256.71 μmol, 85% yield) as a pale yellow liquid. LCMS (ES+): m/z 447.1 [M−tBu+H]⁺.

### Step 3-[2-[[2-[[2-(2,6-dioxo-3-piperidyl)-1,3-dioxo-isoindolin-S-yl]amino]acetyl]amino]ethoxy]propanoic acid (B-57)

3-[2-[[2-[[2-(2,6-dioxo-3-piperidyl)-1,3-dioxo-isoindolin-5-yl]amino]acetyl]amino]ethoxy]propanoic acid (B-57, 100 mg, 210.57 μmol, 82% yield, TFA salt) was synthesized from tert-butyl 3-[2-[2-[[2-(2,6-dioxo-3-piperidyl)-1,3-dioxo-isoindolin-5-yl]amino]acetyl]amino]ethoxy]propanoate (B-56) in a similar fashion to Compound A-26, except using 3 eq. TFA. The material was purified by reverse phase prep HPLC [Prep Method: Column: SunFire C18 (19×150 mm) 5 μm, Mobile phase A: 0.1% TFA in water:MeCN]. LCMS (ES+): m/z 446.9 [M+H]⁺.

### 7-[[2-[[2-(2,6-dioxo-3-piperidyl)-1-oxo-isoindolin-5-yl]amino]acetyl]amino]heptanoic acid (B-62)

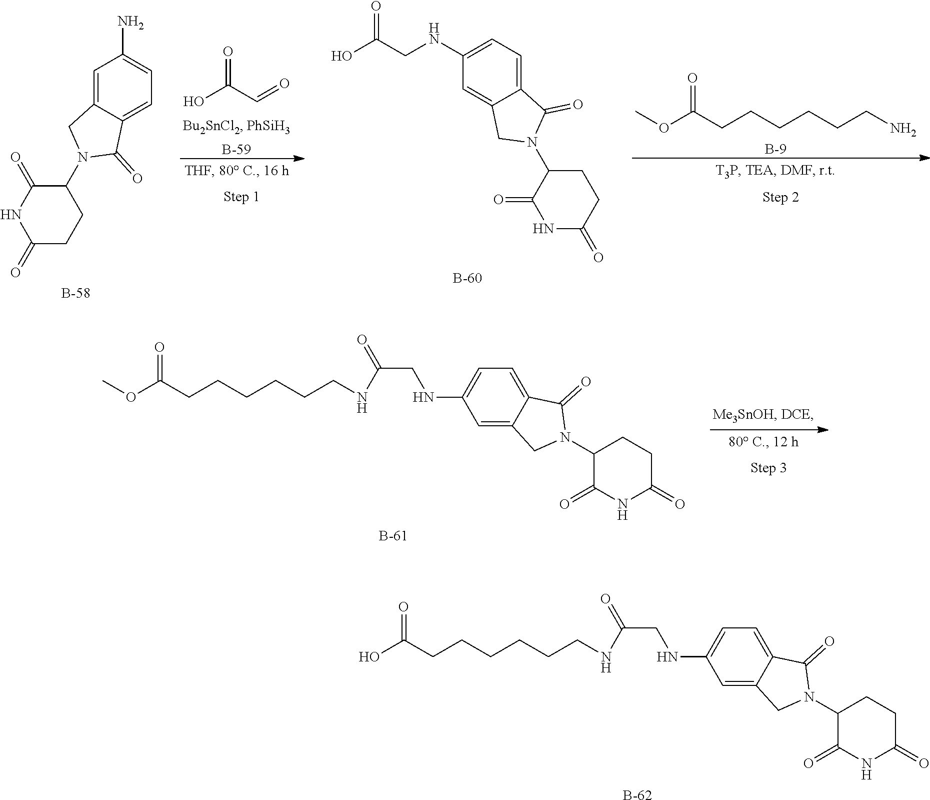

### Step 1: 2-[[2-(2,6-dioxo-3-piperidyl)-1-oxo-isoindolin-5-yl]amino]acetic acid (B-60)

Into a 50 mL single neck round bottom flask containing a well-stirred solution of glyoxylic acid (B-59, 350 mg, 4.73 mmol, 261.19 μL) in anhydrous THF (20 mL) were added 3-(5-amino-1-oxo-isoindolin-2-yl)piperidine-2,6-dione (B-58, 2.45 g, 9.45 mmol), dibutyltin dichloride (1.44 g, 4.73 mmol, 1.06 mL) and phenylsilane (613.89 mg, 5.67 mmol). The reaction mixture was stirred at 80° C. for 16 h. The reaction mixture was cooled to room temperature, the volatiles were removed under reduced pressure and the residue was purified by reverse phase column chromatography (60 g, C18 column, compound eluted with 10-15% acetonitrile in 0.1% TFA in water) to afford 2-[[2-(2,6-dioxo-3-piperidyl)-1-oxo-isoindolin-5-yl]amino]acetic acid (B-60, 450 mg, 1.11 mmol, 23% yield) as an off-white solid. LCMS (ES+): m/z 318.1 [M+H]⁺.

### Step 2: methyl 7-[[2-[[2-(2,6-dioxo-3-piperidyl)-1-oxo-isoindolin-5-yl]amino]acetyl]amino]heptanoate (B-61)

methyl 7-[[2-[[2-(2,6-dioxo-3-piperidyl)-1-oxo-isoindolin-5-yl]amino]acetyl]amino]heptanoate (B-61, 370 mg, 728.93 μmol, 46% yield) was synthesized from 2-[[2-(2,6-dioxo-3-piperidyl)-1-oxo-isoindolin-5-yl]amino]acetic acid (B-60) and methyl 7-aminoheptanoate (B-9) in a similar fashion to Compound B-10, except using 1.5 eq. B-9, 1.5 eq. 1-propanephosphonic anhydride solution (50% in Ethyl acetate). LCMS (ES+): m/z 459.3 [M+H]⁺.

### Step 3: 7-[[2-[[2-(2,6-dioxo-3-piperidyl)-1-oxo-isoindolin-5-yl]amino]acetyl]amino]heptanoic acid (B-62)

7-[[2-[[2-(2,6-dioxo-3-piperidyl)-1-oxo-isoindolin-5-yl]amino]acetyl]amino]heptanoic acid (B-62, 130 mg, 236.91 μmol, 29% yield) was synthesized from methyl 7-[2-[[2-(2,6-dioxo-3-piperidyl)-1-oxo-isoindolin-5-yl]amino]acetyl]amino]heptanoate (B-61) in a similar fashion to Compound B-11, except using 6 eq. trimethyltin hydroxide. LCMS (ES+): m/z 445.2 [M+H]⁺.

### 7-[2-[[2-(2,6-dioxo-3-piperidyl)-1-oxo-isoindolin-4-yl]amino]propanoylamino]heptanoic acid (B-67)

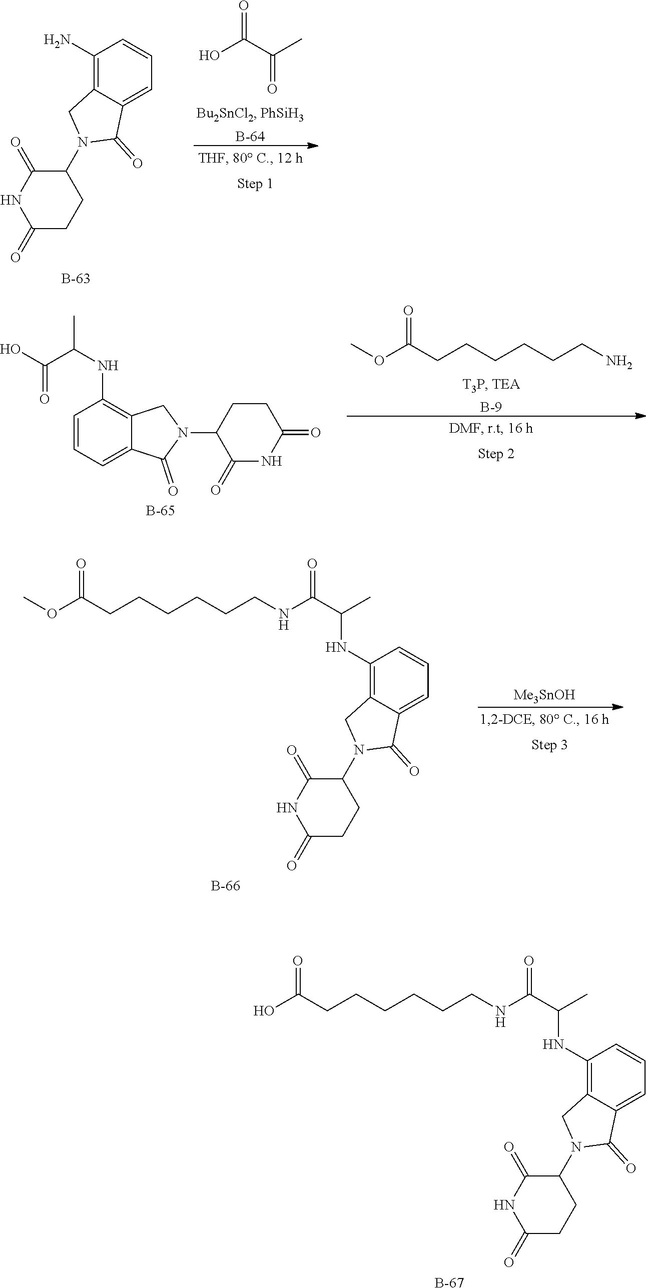

### Step 1: 2-[[2-(2,6-dioxo-3-piperidyl)-1-oxo-isoindolin-4-yl]amino]propanoic acid (B-65)

2-[[2-(2,6-dioxo-3-piperidyl)-1-oxo-isoindolin-4-yl]amino]propanoic acid (B-65, 3.5 g, 72% purity) was synthesized from 3-(4-amino-1-oxo-isoindolin-2-yl)piperidine-2,6-dione (B-63) and 2-oxopropanoic acid (B-64) in a similar fashion to Compound B-60, except using 1.2 eq. B-64. Upon removal of the solvent, the residue was washed with hexanes (2×) and dried under vacuum. The material was used in the next step without further purification. LCMS (ES+): m/z 332.3 [M+H]⁺.

### Step 2: methyl 7-[2-[[2-(2,6-dioxo-3-piperidyl)-1-oxo-isoindolin-4-yl]amino]propanoylamino]heptanoate (B-66)

methyl 7-[2-[[2-(2,6-dioxo-3-piperidyl)-1-oxo-isoindolin-4-yl]amino]propanoylamino]heptanoate (B-66, 900 mg, 1.18 mmol, 27% yield) was synthesized from 2-[[2-(2,6-dioxo-3-piperidyl)-1-oxo-isoindolin-4-yl]amino]propanoic acid (B-65) and methyl 7-aminoheptanoate hydrochloride (B-9) in a similar fashion to Compound B-10, except using 1.3 eq. B-9, 4 eq. triethylamine and 3 eq. 1-propanephosphonic anhydride solution (50% in Ethyl acetate). LCMS (ES+): m/z 472.9 [M+H]⁺.

### Step 3:7-[2-[[2-(2,6-dioxo-3-piperidyl)-1-oxo-isoindolin-4-yl]amino]propanoylamino]heptanoic acid (B-67)

7-[2-[[2-(2,6-dioxo-3-piperidyl)-1-oxo-isoindolin-4-yl]amino]propanoylamino]heptanoic acid (B-67, 95 mg, 196.63 μmol, 33% yield, TFA salt) was synthesized from methyl 7-[2-[[2-(2,6-dioxo-3-piperidyl)-1-oxo-isoindolin-4-yl]amino]propanoylamino]heptanoate (B-66) in a similar fashion to Compound B-11, except using 6 eq. trimethyltin hydroxide. LCMS (ES+): m/z 458.9 [M+H]⁺.

### 3-[3-[[2-(2,6-dioxo-3-piperidyl)-1,3-dioxo-isoindolin-4-yl]propoxy]propanoic acid (B-71)

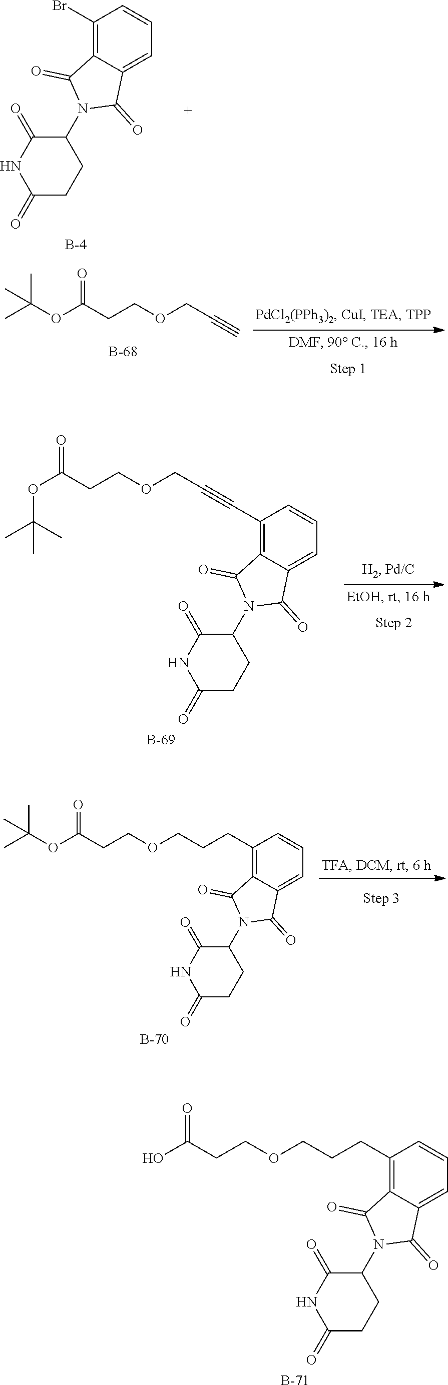

### Step 1: tert-butyl 3-((3-(2-(2,6-dioxopiperidin-3-yl)-1,3-dioxoisoindolin-4-yl) prop-2-yn-1-yloxy)propanoate (B-69)

tert-butyl 3-((3-(2-(2,6-dioxopiperidin-3-yl)-1,3-dioxoisoindolin-4-yl) prop-2-yn-1-yl)oxy)propanoate (B-69, 105 mg, 243.75 μmol, 14% yield) was synthesized from tert-butyl 3-prop-2-ynoxypropanoate (B-68) and 4-bromo-2-(2,6-dioxo-3-piperidyl)isoindoline-1,3-dione (B-4) in a similar fashion to Compound B-5, except using 1.2 eq. B-4 and 0.2 eq. Cul. LCMS (ES+): m/z 385.2 [M−tBu+H]⁺.

### Step 2: tert-butyl 3-[3-[[2-(2,6-dioxo-3-piperidyl)-1,3-dioxo-isoindolin-4-yl]propoxy]propanoate (B-70)

tert-butyl 3-[3-[[2-(2,6-dioxo-3-piperidyl)-1,3-dioxo-isoindolin-4-yl]propoxy]propanoate (B-70, 90 mg, 180.21 μmol, 76% yield) was synthesized from tert-butyl 3-[3-[1,3-dioxo-2-(2-oxo-3-piperidyl)isoindolin-4-yl]prop-2-ynoxy]propanoate (B-69) in a similar fashion to Compound B-6, except using 0.1 eq. palladium (10% on carbon). LCMS (ES+): m/z 444.9 [M+H]⁺.

### Step 3: 3-[3-[[2-(2,6-dioxo-3-piperidyl)-1,3-dioxo-isoindolin-4-yl]propoxy]propanoic acid (B-71)

3-[3-[[2-(2,6-dioxo-3-piperidyl)-1,3-dioxo-isoindolin-4-yl]propoxy]propanoic acid (B-71, 60 mg, 77% yield, TFA salt) was synthesized from tert-butyl 3-[3-[[2-(2,6-dioxo-3-piperidyl)-1,3-dioxo-isoindolin-4-yl]propoxy]propanoate (B-70) in a similar fashion to Compound A-26, except using 5 eq. TFA.

### 4-[[2-[[2-(2,6-dioxo-3-piperidyl)-1-oxo-isoindolin-4-yl]amino]acetyl]amino]butanoic acid (B-74)

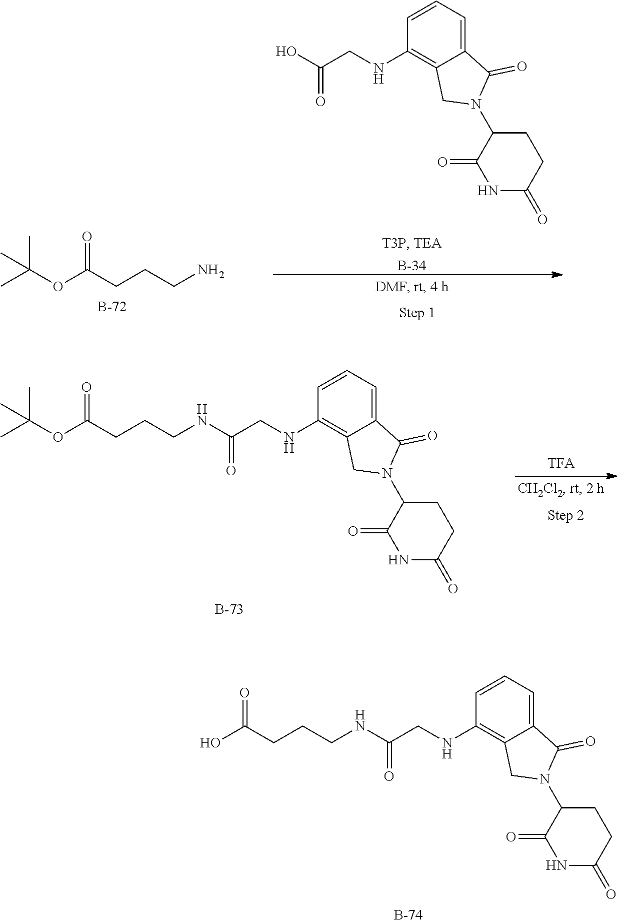

### Step 1: tert-butyl 4-[[2-[[2-(2,6-dioxo-3-piperidyl)-1-oxo-isoindolin-4-yl]amino]acetyl]amino]butanoate (B-73)

tert-butyl 4-[2-[[2-(2,6-dioxo-3-piperidyl)-1-oxo-isoindolin-4-yl]amino]acetyl]amino]butanoate (B-73, 0.3 g, 416.59 μmol, 26% yield) was synthesized from 2-[[2-(2,6-dioxo-3-piperidyl)-1-oxo-isoindolin-4-yl]amino]acetic acid (B-34) and tert-butyl 4-aminobutanoate (B-72) in a similar fashion to Compound B-10, except using 2.5 eq. triethylamine, 1.6 eq. 1-propanephosphonic anhydride solution (50% in Ethyl acetate) and 1.2 eq. B-72. LCMS (ES+): m/z 403.2 [M−tBu+H]⁺.

### Step 2: 4-[[2-[[2-(2,6-dioxo-3-piperidyl)-1-oxo-isoindolin-4-yl]amino]acetyl]amino]butanoic acid (B-74)

4-[2-[[2-(2,6-dioxo-3-piperidyl)-1-oxo-isoindolin-4-yl]amino]acetyl]amino]butanoic acid (B-74, 0.080 g, 174.43 μmol, 20% yield, TFA salt) was synthesized from tert-butyl 4-[[2-[[2-(2,6-dioxo-3-piperidyl)-1-oxo-isoindolin-4-yl]amino]acetyl]amino]butanoate (B-73) in a similar fashion to Compound A-26, except using 6 eq. TFA. The material was purified by reverse-phase preparative HPLC (Column: SunFire C18 (19×150 mm) 5 μm, Mobile phase A: 0.1% TFA in water:MeCN). LCMS (ES+): m/z 403.2 [M+H]⁺.

### 2-[[2-(2,6-dioxo-3-piperidyl)-3-oxo-isoindolin-4-yl]amino]acetic acid (B-76)

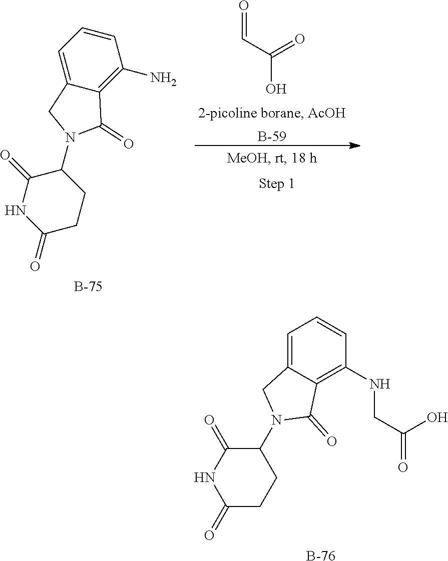

### Step 1: 2-□[2-(2,6-dioxo-3-piperidyl)-3-oxo-isoindolin-4-yl]amino]acetic acid (B-76)

Into a 50 mL single neck round bottom flask containing a well-stirred solution of 3-(7-amino-1-oxo-isoindolin-2-yl)piperidine-2,6-dione (B-75, 2 g, 7.71 mmol) and glyoxylic acid (B-59, 1.14 g, 15.43 mmol, 852.43 μL) in methanol (20 mL) was added acetic acid (2.32 g, 38.57 mmol, 2.21 mL) followed by portionwise addition of 2-picoline borane complex (1.65 g, 15.43 mmol) at 0° C. The reaction mixture was stirred at room temperature for 18 h. Upon completion, water (10 mL) was added to the reaction mixture and solvent removed under reduced pressure to afford the crude product, which was purified by reverse phase prep HPLC [Purification method: Column: SunFire prep OBD 19×50 mm (5 μm), Mobile phase A: 0.1% TFA in water and Mobile phase B: MeCN] to afford 2-[[2-(2,6-dioxo-3-piperidyl)-3-oxo-isoindolin-4-yl]amino]acetic acid (B-76, 130 mg, 406.72 μmol, 5% yield, TFA salt) as an off-white solid. LCMS (ES+): m/z 318.1 [M+H]⁺.

### 6-[4-[[2-(2,6-dioxo-3-piperidyl)-1,3-dioxo-isoindolin-4-yl]amino]-1-piperidyl]hexanoic acid (B-82)

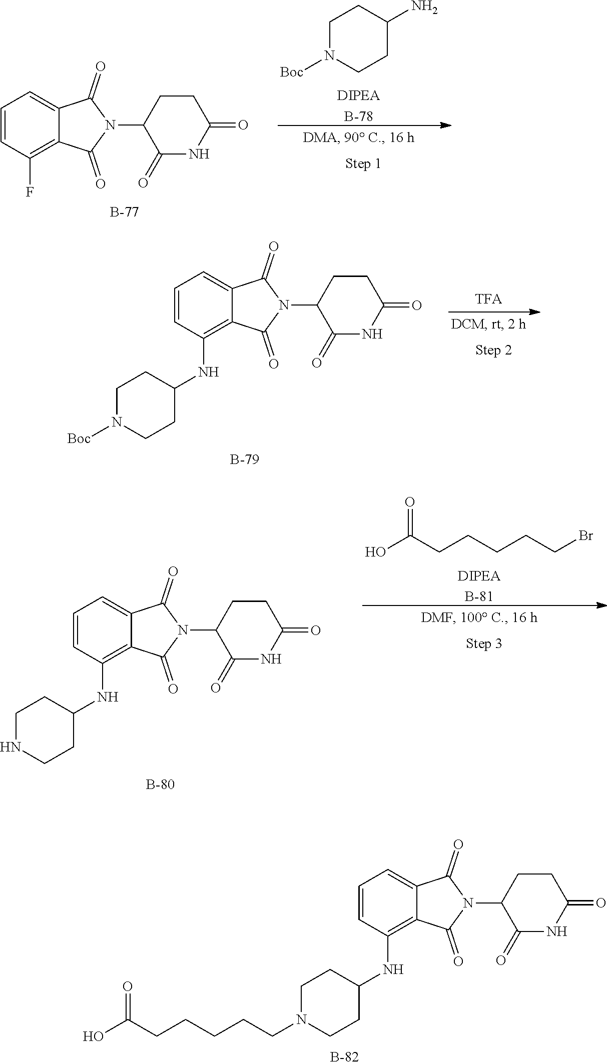

### Step 1: ter-butyl 4-[[2-(2,6-dioxo-3-piperidyl)-1,3-dioxo-isoindolin-4-yl]amino]piperidine-1-carboxylate (B-79)

Into a 25 mL pressure tube containing a well-stirred solution of 2-(2,6-dioxo-3-piperidyl)-4-fluoro-isoindoline-1,3-dione (B-77, 0.7 g, 2.53 mmol) in dimethylacetamide (10 mL) at room temperature were added tert-butyl 4-aminopiperidine-1-carboxylate (B-78, 507.54 mg, 2.53 mmol) followed by N,N-diisopropylethylamine (982.59 mg, 7.60 mmol, 1.32 mL). The reaction mixture was stirred at 90° C. for 16 h. The mixture was cooled to room temperature, diluted with water (30 mL) and filtered. The solid was washed with water (30 mL) followed by petroleum ether (30 mL) to afford a tert-butyl 4-[[2-(2,6-dioxo-3-piperidyl)-1,3-dioxo-isoindolin-4-yl]amino]piperidine-1-carboxylate (B-79, 0.6 g, 802.95 μmol, 32% yield) as a brown solid. LCMS (ES+): m/z 356.9 [M−Boc+H]⁺.

### Step 2: 2-(2,6-dioxo-3-piperidyl)-4-(4-piperidylamino)isoindoline-1,3-dione (B-80)

2-(2,6-dioxo-3-piperidyl)-4-(4-piperidylamino)isoindoline-1,3-dione (B-80, 0.4 g, 583.42 μmol, 73% yield, 69% purity, TFA salt) was synthesized from tert-butyl 4-[[2-(2,6-dioxo-3-piperidyl)-1,3-dioxo-isoindolin-4-yl]amino]piperidine-1-carboxylate (B-79) in a similar fashion to Compound A-62, except using 10 eq. TFA. The material was triturated with diethyl ether. LCMS (ES+): m/z 357.0 [M+H]⁺.

### Step 3: 6-[4-[[2-(2,6-dioxo-3-piperidyl)-1,3-dioxo-isoindolin-4-yl]amino]-1-piperidyl]hexanoic acid (B-82)

Into a 25 mL pressure tube containing a well-stirred solution of 2-(2,6-dioxo-3-piperidyl)-4-(4-piperidylamino)isoindoline-1,3-dione (B-80, 0.4 g, 583.42 μmol, 72.66% yield, 69% purity) in DMF (5 mL) were added 6-bromohexanoic acid (B-81, 113.80 mg, 583.42 μmol) and N,N-diisopropylethylamine (377.02 mg, 2.92 mmol, 508.11 L) at room temperature. The reaction mixture was stirred at 100° C. for 16 h. The mixture was cooled to room temperature, diluted with water (25 mL) and extracted with ethyl acetate (3×25 mL). The combined organic layer was washed with water (15 mL) and brine (15 mL), dried over anhydrous sodium sulfate and filtered. The solvent was removed under reduced pressure to afford 6-[4-[[2-(2,6-dioxo-3-piperidyl)-1,3-dioxo-isoindolin-4-yl]amino]-1-piperidyl]hexanoic acid (B-82, 0.15 g, 304.96 μmol, 52% yield) as yellow solid. The material was used in the next step without further purification. LCMS (ES+): m/z 471.2 [M+H]⁺.

### 6-[4-[[2-(2,6-dioxo-3-piperidyl)-1-oxo-isoindolin-4-yl]amino]-1-piperidyl]hexanoic acid (B-84)

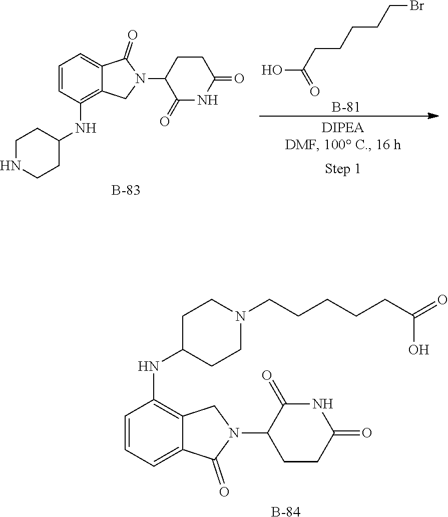

### Step 1: 6-[4-[[2-(2,6-dioxo-3-piperidyl)-1-oxo-isoindolin-4-yl]amino]-1-piperidyl]hexanoic acid (B-84)

6-[4-[[2-(2,6-dioxo-3-piperidyl)-1-oxo-isoindolin-4-yl]amino]-1-piperidyl]hexanoic acid (B-84, 80 mg, 154.21 μmol, 11% yield, TFA salt) was synthesized from 3-[1-oxo-4-(4-piperidyl amino)isoindolin-2-yl]piperidine-2,6-dione (B-83) and 6-bromohexanoic acid (B-81) in a similar fashion to Compound B-82, except using 1.2 eq. B-81. Upon completion, the volatiles were removed under reduced pressure and the residue purified by reverse phase prep HPLC [Purification method: Column: SunFire-C18 100 mm, Mobile phase A: 0.1% TFA in water, Mobile phase B: MeCN; Flow Rate: 15.0 mL/min]. LCMS (ES+): m/z 457.0 [M+H]⁺.

### 3-[4-[[2-(2,6-dioxo-3-piperidyl)-1-oxo-isoindolin-4-yl]amino]methyl]pyrazol-1-yl]propanoic acid (B-88)

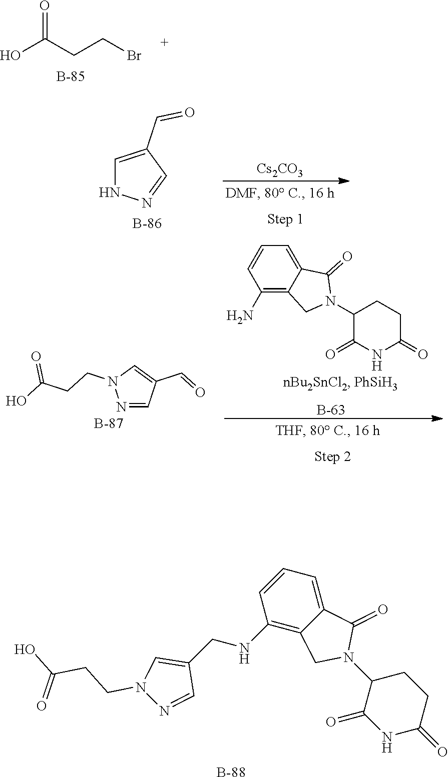

### Step 1: 3-(4-formylpyrazol-1-yl)propanoic acid (B-87)

Into a 100 mL three neck round bottom flask containing a well-stirred solution of 1H-pyrazole-4-carbaldehyde (B-86, 1.0 g, 10.41 mmol) in DMF (10 mL) was added cesium carbonate (6.78 g, 20.81 mmol) and the suspension was stirred for 5 min. Then, 3-bromopropanoic acid (B-85, 3.18 g, 20.81 mmol, 2.15 mL) was added and the reaction mixture was stirred at 80° C. for 16 h. The reaction was concentrated under reduced pressure and the residue was diluted with water (50 mL) and acidified using 2N HCl and extracted with Ethyl acetate (3×50 mL). The combined organic layer was dried over anhydrous sodium sulfate, filtered and concentrated under reduced pressure to afford 3-(4-formylpyrazol-1-yl)propanoic acid (B-87, 1.8 g, 4.50 mmol, 43% yield) as thick red liquid. LCMS (ES+): m/z 169.0 [M+H]⁺.

### Step 2: 3-[4-[[[2-(2,6-dioxo-3-piperidyl)-1-oxo-isoindolin-4-yl]amino]methyl]pyrazol-1-yl]propanoic acid (B-88)

3-[4-[[[2-(2,6-dioxo-3-piperidyl)-1-oxo-isoindolin-4-yl]amino]methyl]pyrazol-1-yl]propanoic acid (B-88, 40 mg, 94.50 μmol, 25% yield) was synthesized from 3-(4-amino-1-oxo-isoindolin-2-yl)piperidine-2,6-dione (B-63) and 3-(4-formylpyrazol-1-yl)propanoic acid (B-87) in a similar fashion to Compound B-60, except using 1.2 eq. B-87. LCMS (ES+): m/z 412.1 [M+H]⁺.

### 2-[4-[[2-(2,6-dioxo-3-piperidyl)-1-oxo-isoindolin-5-yl]-1-piperidyl]acetic acid (B-90)

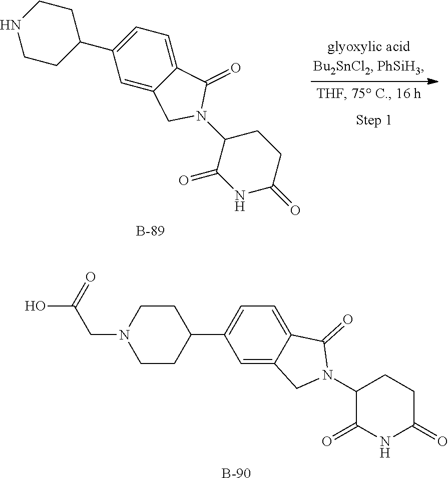

### Step 1: 2-[4-[[2-(2,6-dioxo-3-piperidyl)-1-oxo-isoindolin-5-yl]-1-piperidyl]acetic acid (B-90)

2-[4-[[2-(2,6-dioxo-3-piperidyl)-1-oxo-isoindolin-5-yl]-1-piperidyl]acetic acid (B-90, 102 mg, 262.72 μmol, 86% yield) was synthesized from 3-[1-oxo-5-(4-piperidyl)isoindolin-2-yl]piperidine-2,6-dione (B-89) and glyoxylic acid monohydrate in a similar fashion to Compound B-60, except using 1.5 eq. glyoxylic acid monohydrate. Upon completion, the solvent was evaporated under reduced pressure and the residue was triturated ethyl acetate and diethyl ether, filtered and dried. The material was used in the next step without further purification. LCMS (ES+): m/z 385.9 [M+H]⁺.

### 3-(4-((2-(2,6-dioxopiperidin-3-yl)-1-oxoisoindolin-4-yl)amino)piperidin-1-yl)propanoic acid (B-92)

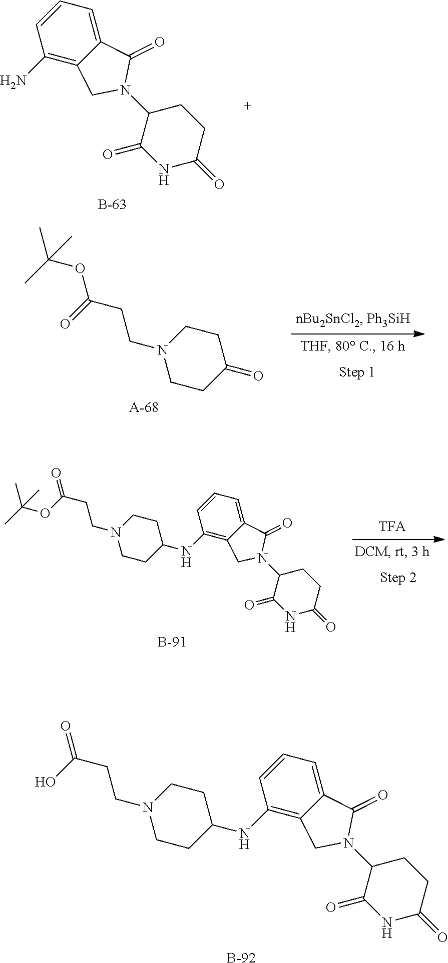

### Step 1: tert-butyl 3-[4-[[2-(2,6-dioxo-3-piperidyl)-1-oxo-isoindolin-4-yl]amino]-1-piperidyl]propanoate (B-91)

tert-butyl 3-[4-[[2-(2,6-dioxo-3-piperidyl)-1-oxo-isoindolin-4-yl]amino]-1-piperidyl]propanoate (B-91, 510 mg, 1.02 mmol, 75% yield) was synthesized from 3-(4-amino-1-oxo-isoindolin-2-yl)piperidine-2,6-dione (B-63) and tert-butyl3-(4-oxo-1-piperidyl)propanoate (A-68) in a similar fashion to Compound B-60, except using 1.5 eq. A-68 and 1.2 eq. phenyl silane. LCMS (ES+): m/z 471.6 [M+H]⁺.

### Step 2: 3-(4-((2-(2,6-dioxopiperidin-3-yl)-1-oxoisoindolin-4-yl)amino)piperidin-1-yl)propanoic acid (B-92)

3-(4-((2-(2,6-dioxopiperidin-3-yl)-1-oxoisoindolin-4-yl)amino)piperidin-1-yl)propanoic acid (B-92, 300 mg, 89% yield) was synthesized from tert-butyl 3-[4-[[2-(2,6-dioxo-3-piperidyl)-1-oxo-isoindolin-4-yl]amino]-1-piperidyl]propanoate (B-91) in a similar fashion to Compound A-26. LCMS (ES+): m/z 415.0 [M+H]⁺.

### 3-[[2-(2,6-dioxo-3-piperidyl)-1-oxo-isoindolin-5-yl]propanoic acid (B-95)

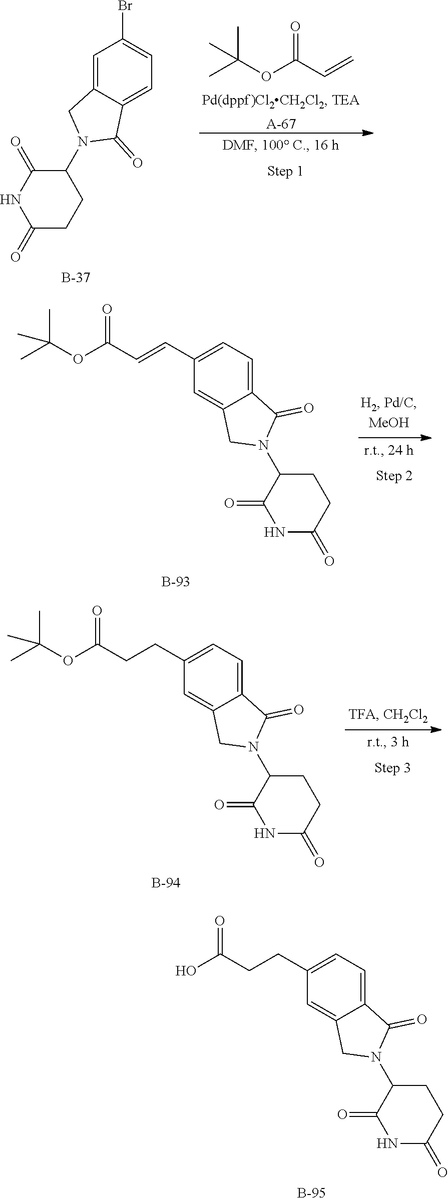

### Step 1: tert-butyl (E)-3-(2-(2,6-dioxopiperidin-3-yl)-1-oxoisoindolin-5-yl)acrylate (B-93)

Into a 100 mL pressure tube containing a well-stirred solution of 3-(5-bromo-1-oxo-isoindolin-2-yl)piperidine-2,6-dione (B-37, 2.00 g, 3.09 mmol) in anhydrous DMF (20 mL) were added tert-butyl prop-2-enoate (A-67, 594.95 mg, 4.64 mmol, 673.78 μL) and triethylamine (939.43 mg, 9.28 mmol, 1.29 mL) at room temperature under nitrogen atmosphere. The reaction mixture was purged by bubbling nitrogen through the solution for 5 min before adding 1,1′-Bis(diphenylphosphino)ferrocene palladium(II)chloride dichloromethane complex (252.72 mg, 0.309 mmol). The tube was sealed, and the suspension was heated at 100° C. for 16 h. Upon completion, the mixture was cooled to room temperature and poured into cold water (200 mL) and extracted with Ethyl acetate (2×100 mL). The combined organic layer was dried over anhydrous sodium sulfate. The solvent was removed under reduced pressure and the residue purified by flash silica-gel (230-400 mesh) column chromatography (0-100% Ethyl acetate in petroleum ether) to obtain tert-butyl (E)-3-[[2-(2,6-dioxo-3-piperidyl)-1-oxo-isoindolin-5-yl]prop-2-enoate (B-93, 600 mg, 1.51 mmol, 49% yield) as a light brown solid. LCMS (ES+): m/z 371.3 [M+H]⁺.

### Step 2: tert-butyl 3-(2-(2,6-dioxopiperidin-3-yl)-1-oxoisoindolin-5-yl)propanoate (B-94)

tert-butyl 3-[[2-(2,6-dioxo-3-piperidyl)-1-oxo-isoindolin-5-yl]propanoate (B-94, 300 mg) was synthesized from tert-butyl (E)-3-[[2-(2,6-dioxo-3-piperidyl)-1-oxo-isoindolin-5-yl]prop-2-enoate (B-93) in a similar fashion to Compound B-6, except using 0.1 eq. Palladium (10% on carbon, dry) using a hydrogen bladder. After triturating with diethyl ether, the material was used in the next step without further purification. LCMS (ES+): m/z 373.3 [M+H]⁺.

### Step 3: 3-[[2-(2,6-dioxo-3-piperidyl)-1-oxo-isoindolin-5-yl]propanoic acid (B-95)

3-[[2-(2,6-dioxo-3-piperidyl)-1-oxo-isoindolin-5-yl]propanoic acid (B-95, 250 mg, 0.790 mmol, 98% yield, TFA salt) was synthesized from tert-butyl 3-[[2-(2,6-dioxo-3-piperidyl)-1-oxo-isoindolin-5-yl]propanoate (B-94) in a similar fashion to Compound A-26, except using 16 eq. TFA. LCMS (ES+): m/z 316.9 [M+H]⁺.

### 3-[1-(2,6-dioxo-3-piperidyl)-3-methyl-2-oxo-benzimidazol-5-yl]propanoic acid (B-104)

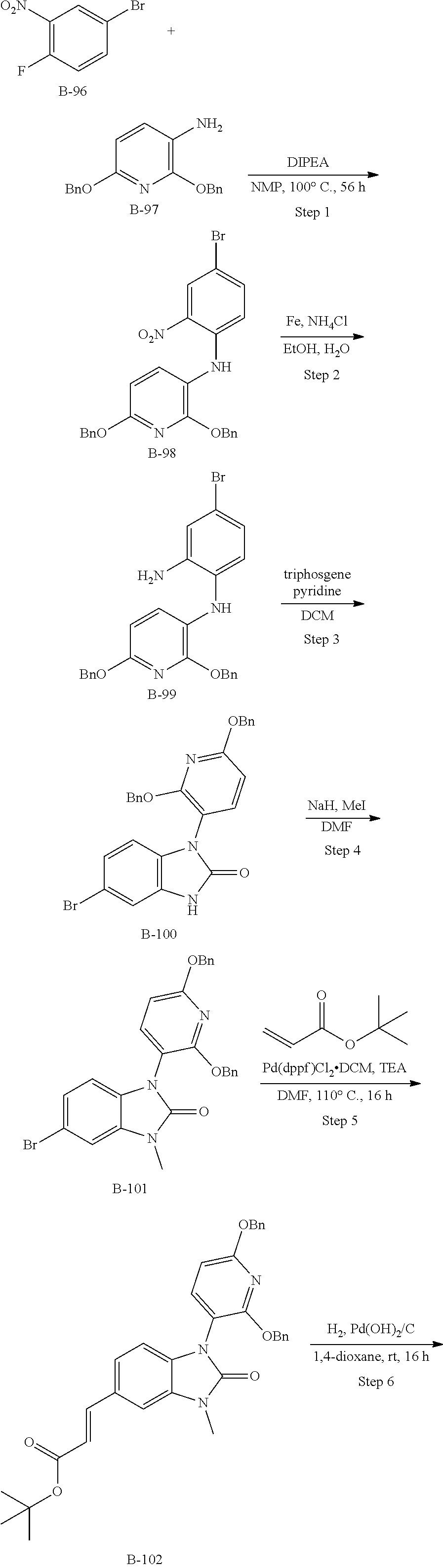

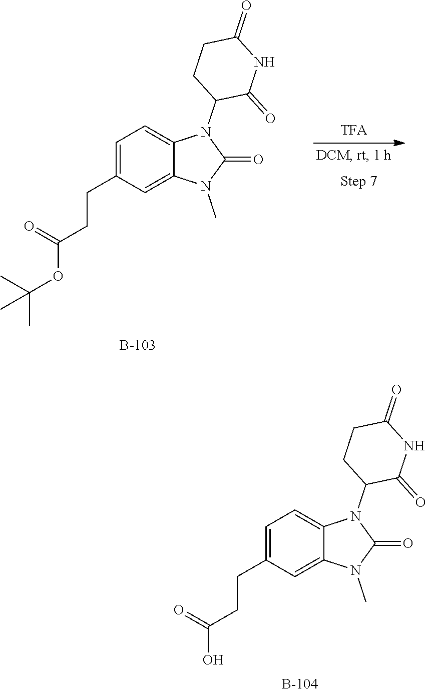 [SMILES: Cn1c(=O)n(C2CCC(=O)NC2=O)c2ccc(CCC(=O)OC(C)(C)C)cc21 ; Cn1c(=O)n(C2CCC(=O)NC2=O)c2ccc(CCC(=O)O)cc21]

### Step 1: 2,6-dibenzyloxy-N-(4-bromo-2-nitro-phenyl)pyridin-3-amine (B-98)

To a stirred solution of 4-bromo-1-fluoro-2-nitro-benzene (B-96, 25 g, 113.64 mmol, 13.97 mL) in NMP (250 mL) in a sealed tube was added 2,6-dibenzyloxypyridin-3-amine (B-97, 34.81 g, 113.64 mmol) followed by DIPEA (73.43 g, 568.19 mmol, 98.97 mL) and stirred for 56 h at 100° C. The solvent was removed, and the residue diluted with DCM (3×800 mL) and Water (500 mL). The organic layer was dried over sodium sulfate, filtered and solvent removed under reduced pressure. The residue was slurried with a mixture of Petroleum ether and Ethyl acetate and stirred for 30 min. The precipitate was filtered and dried under vacuum to afford 2,6-dibenzyloxy-N-(4-bromo-2-nitro-phenyl)pyridin-3-amine (B-98, 30 g, 57.86 mmol, 51% yield) as a yellow solid. LCMS (ES+): m/z 505.9 [M+H]⁺.

### Step 2: 4-bromo-N1-(2,6-dibenzyloxy-3-pyridyl)benzene-1,2-diamine (B-99)

Into a 2 L two neck round bottom flask containing a well-stirred solution of 2,6-dibenzyloxy-N-(4-bromo-2-nitro-phenyl)pyridin-3-amine (B-98, 31 g, 45.04 mmol) in Water (310 mL) and Ethanol (310 mL) was added iron powder (15.09 g, 270.25 mmol) and ammonium chloride (14.46 g, 270.25 mmol) at room temperature. The reaction mixture was heated at 70° C. for 16 h. The mixture was filtered through Celite, washing with DCM (500 mL). The volatiles were removed and the material extracted with DCM (1 L). The organic layer was dried over sodium sulfate, filtered and solvent removed to afford 4-bromo-N1-(2,6-dibenzyloxy-3-pyridyl)benzene-1,2-diamine (B-99, 22 g, 33.98 mmol, 75% yield) as a thick brown colored mass. The material was used in the next step without further purification. LCMS (ES+): m/z 478.0 [M+H]⁺.

### Step 3: 6-bromo-3-(2,6-dibenzyloxy-3-pyridyl)-1H-benzimidazol-2-one (B-100)

To a solution of 4-bromo-N1-(2,6-dibenzyloxy-3-pyridyl)benzene-1,2-diamine (B-99, 28 g, 42.32 mmol) in DCM (70 mL) was added pyridine (33.48 g, 423.21 mmol, 34.23 mL). After 5 min, the mixture was cooled to 0° C. and triphosgene (25.12 g, 84.64 mmol) dissolved in 10 mL DCM was added via an addition funnel. After 16 h, the reaction was quenched with ice while stirring and the product was extracted into DCM (3×250 mL). The organic layer was concentrated and the residue purified via flash column chromatography (35% Ethyl acetate:Hexanes) to afford 6-bromo-3-(2,6-dibenzyloxy-3-pyridyl)-1H-benzimidazol-2-one (B-100, 18 g, 35.47 mmol, 84% yield) as an off white solid. LCMS (ES+): m/z 503.1 [M+H]⁺.

### Step 4: 5-bromo-1-(2,6-dibenzyloxy-3-pyridyl)-3-methyl-benzimidazol-2-one (B-101)

5-bromo-1-(2,6-dibenzyloxy-3-pyridyl)-3-methyl-benzimidazol-2-one (B-101, 20 g, 38.38 mmol, 96% yield) was synthesized from 6-bromo-3-(2,6-dibenzyloxy-3-pyridyl)-1H-benzimidazol-2-one (B-100) and iodomethane in a similar fashion to Compound B-3, except sing 1.5 eq. NaH and 2 eq. iodomethane. The reaction was quenched with saturated ammonium chloride solution, and the material was used in the next step without further purification. LCMS (ES+): m/z 516.0 [M+H]⁺.

### Step 5: tert-butyl (E)-3-[1-(2, 6-dibenzyloxy-3-pyridyl)-3-methyl-2-oxo-benzimidazol-5-yl]prop-2-enoate (B-102)

tert-butyl (E)-3-[1-(2, 6-dibenzyloxy-3-pyridyl)-3-methyl-2-oxo-benzimidazol-5-yl]prop-2-enoate (B-102, 145 mg, 174.19 μmol, 60% yield) was synthesized from 5-bromo-1-(2,6-dibenzyloxy-3-pyridyl)-3-methyl-benzimidazol-2-one (B-101) and tert-butyl prop-2-enoate (A-67) in a similar fashion to Compound B-93, except using 4 eq. A-67 and 5 eq. triethylamine. The reaction was heated at 110° C. for 16 h. Upon completion, the reaction was filtered through Celite, washing with ethyl acetate. The filtrate was washed with water (2×) and brine, dried over anhydrous sodium sulfate and filtered. The solvent was removed under reduced pressure and the residue purified by flash silica gel column chromatography (5-10% Ethyl acetate in petroleum ether). MS (ESI) m/z=564.30 [M+H]⁺.

### Step 6: tert-butyl 3-(1-(2, 6-dioxopiperidin-3-yl)-3-methyl-2-oxo-2,3-dihydro-1H-benzo[d]imidazol-5-yl))propanoate (B-103)

Into a 100 mL single neck round bottom flask containing a well-stirred solution of tert-butyl (E)-3-(1-(2,6-bis(benzyloxy)pyridin-3-yl)-3-methyl-2-oxo-2,3-dihydro-1H-benzo[d]imidazol-5-yl)acrylate (B-102, 145 mg, 257.26 μmol) in 1,4-dioxane (50 mL) was added palladium hydroxide (10% on carbon) (78.0 mg, 55.55 μmol). The reaction mixture was stirred under hydrogen atmosphere at 1 atm for 16 h. The reaction mixture was filtered through Celite and washed with Ethyl acetate (50 mL). The solvent was removed under reduced pressure to obtain tert-butyl 3-(1-(2,6-dioxopiperidin-3-yl)-3-methyl-2-oxo-2,3-dihydro-1H-benzo[d]imidazol-5-yl)propanoate (B-103, 100 mg, 212.93 μmol, 83% yield) as a pale green solid. The material was used in the next step without further purification, MS (ESI): m/z 388.40 [M+H]⁺.

### Step 7: 3-[1-(2,6-dioxo-3-piperidyl)-3-methyl-2-oxo-benzimidazol-5-yl]propanoic acid (B-104)

3-[1-(2,6-dioxo-3-piperidyl)-3-methyl-2-oxo-benzimidazol-5-yl]propanoic acid (B-104, 150 mg, 253.47 μmol, 98% yield) was synthesized from tert-butyl 3-(1-(2,6-dioxopiperidin-3-yl)-3-methyl-2-oxo-2,3-dihydro-1H-benzo[d]imidazol-5-yl)propanoate (B-103) in a similar fashion to Compound A-26, except using 4 eq. TFA. MS (ESI): m/z 331.9 [M+H]⁺.

### 3-[3-methyl-2-oxo-4-(4-piperidyl)benzimidazol-1-yl]piperidine-2,6-dione (B-113)

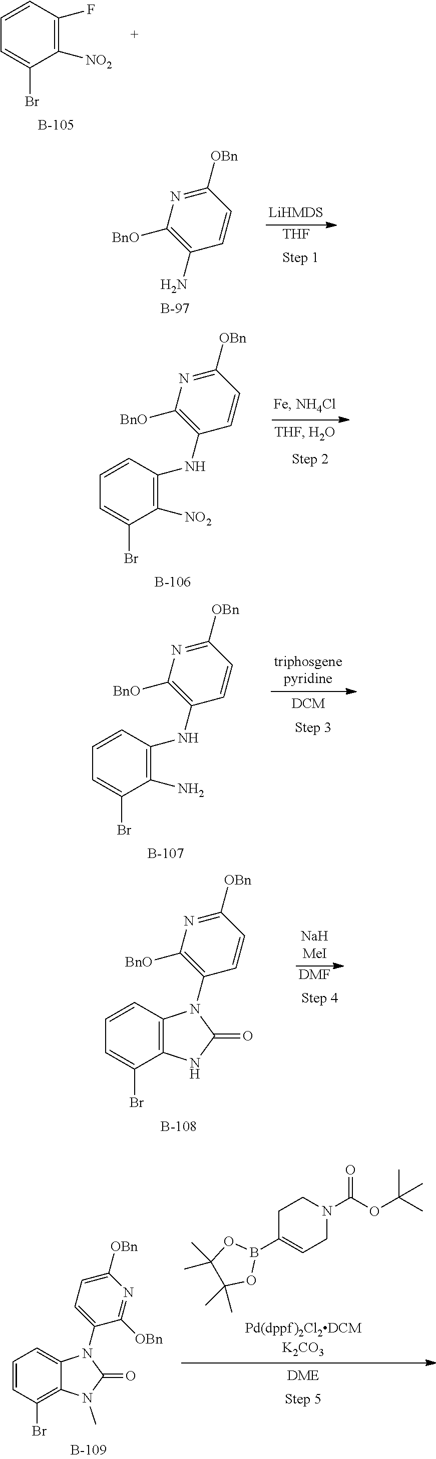

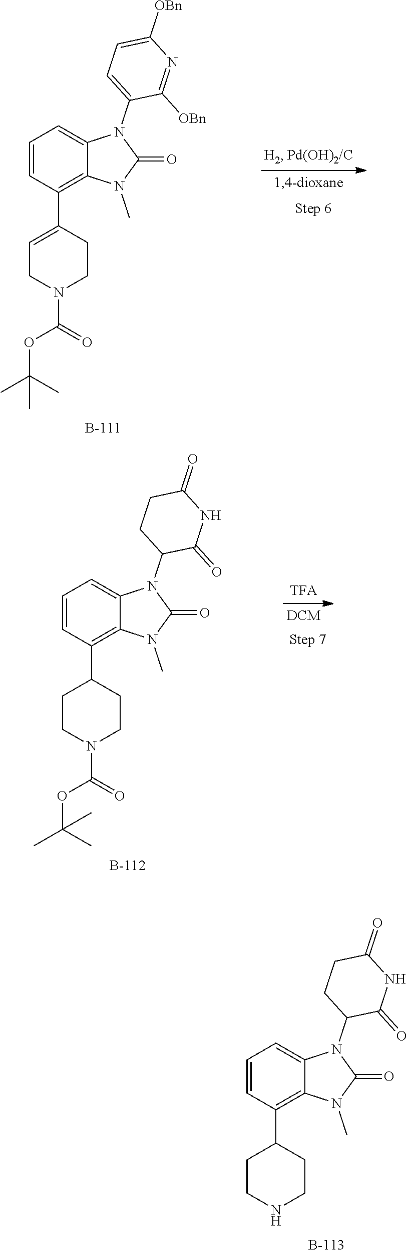 [SMILES: Cn1c(=O)n(-c2ccc(OCc3ccccc3)nc2OCc2ccccc2)c2cccc(C3=CCN(C(=O)OC(C)(C)C)CC3)c21 ; Cn1c(=O)n(C2CCC(=O)NC2=O)c2cccc(C3CCN(C(=O)OC(C)(C)C)CC3)c21 ; Cn1c(=O)n(C2CCC(=O)NC2=O)c2cccc(C3CCNCC3)c21]

### Step 1: 2,6-dibenzyloxy-N-(3-bromo-2-nitro-phenyl)pyridin-3-amine (B-106)

To a solution of 2,6-dibenzyloxypyridin-3-amine (B-97, 3 g, 9.79 mmol) in THF (100 mL) was added Lithium bis(trimethylsilyl)amide (1M in THF) (3.60 g, 21.54 mmol, 22 mL) at 0° C. via dropwise addition. The reaction mixture was stirred for 15 min at 0° C. before 1-bromo-3-fluoro-2-nitro-benzene (B-105, 2.37 g, 10.77 mmol) dissolved in 2 mL THF was added. After 4 h at room temperature, the reaction was quenched with water (50 mL) at 0° C. and extracted with ethyl acetate (2×100 mL). The organic layer was separated, dried over sodium sulfate, filtered and concentrated. The residue was purified by Isolera using 230-400 silica. At 15-20% Ethyl acetate in petroleum ether to afford 2,6-dibenzyloxy-N-(3-bromo-2-nitro-phenyl)pyridin-3-amine (B-106, 3.3 g, 4.30 mmol, 44% yield) as brown viscous liquid. LCMS (ES−): m/z 404.0 [M−H]⁻.

### Step 2: 3-bromo-N1-(2,6-dibenzyloxy-3-pyridyl)benzene-1,2-diamine (B-107)

A mixture of 2,6-dibenzyloxy-N-(3-bromo-2-nitro-phenyl)pyridin-3-amine (B-106, 1 g, 1.97 mmol), Iron powder (1.10 g, 19.75 mmol, 140.32 μL), and Ammonium Chloride (1.06 g, 19.75 mmol, 690.47 μL) in THF (40 mL) and Water (20 mL) was heated at 70° C. for 7 h. Upon completion, the reaction mixture was passed through Celite and 20 mL water was added to the filtrate. The product was extracted with ethyl acetate (3×50 mL). The combined organic layer was dried over sodium sulfate and evaporated to afford 3-bromo-N1-(2,6-dibenzyloxy-3-pyridyl)benzene-1,2-diamine (B-107, 1.00 g, 1.53 mmol, 77% yield, 73% purity). The material was used in the next step without further purification. LCMS (ES+): m/z 476.21 [M+H]⁺.

### Step 3:7-bromo-3-(2,6-dibenzyloxy-3-pyridyl)-1H-benzimidazol-2-one (B-108)

7-bromo-3-(2,6-dibenzyloxy-3-pyridyl)-1H-benzimidazol-2-one (B-108, 1.9 g, 3.44 mmol, 66% yield) was synthesized from 3-bromo-N1-(2,6-dibenzyloxy-3-pyridyl)benzene-1,2-diamine (B-107) in a similar fashion to Compound B-100, except using 1.5 eq. triphosgene Upon completion, the reaction mixture was diluted with Ethyl acetate and water. The organic layer was washed with 10% sodium bicarbonate and brine, dried over sodium sulfate, filtered and concentrated. The material was used in the next step without further purification. LCMS (ES+): m/z 503.9 [M+H]⁺.

### Step 4:4-bromo-1-(2,6-dibenzyloxy-3-pyridyl)-3-methyl-benzimidazol-2-one (B-109)

4-bromo-1-(2,6-dibenzyloxy-3-pyridyl)-3-methyl-benzimidazol-2-one (B-109, 2.8 g, 5.15 mmol, 91% yield) was synthesized from 7-bromo-3-(2,6-dibenzyloxy-3-pyridyl)-1H-benzimidazol-2-one (B-108) and methyl iodide in a similar fashion to Compound B-3, except using 1.5 eq. NaH and 1.5 eq. methyl iodide. 1HNMR (400 MHz, DMSO-d6): δ 7.83-7.81 (d, J=8 Hz, 1H), 7.45-7.43 (m, 2H), 7.40-7.33 (m, 3H), 7.27-7.24 (m, 6H), 6.92 (t, J=8 Hz, 1H), 6.69-6.67 (d, J=8 Hz, 1H), 6.63-6.61 (d, J=8 Hz, 1H), 5.43-5.33 (m, 4H), 3.67 (s, 3H). LCMS (ES+); m/z 516.1 [M+H]⁺.

### Step 5: tert-butyl 4-[1-(2,6-dibenzyloxy-3-pyridyl)-3-methyl-2-oxo-benzimidazol-4-yl]-3,6-dihydro-2H-pyridine-1-carboxylate (B-111)

Into a 10 mL pressure tube containing a well-stirred-solution of 4-bromo-1-(2,6-dibenzyloxy-3-pyridyl)-3-methyl-benzimidazol-2-one (B-109, 100 mg, 193.65 μmol) and tert-butyl 4-(4,4,5,5-tetramethyl-1,3,2-dioxaborolan-2-yl)-3,6-dihydro-2H-pyridine-1-carboxylate (B-110, 59.88 mg, 193.65 μmol) in 1,2-dimethoxyethane (3 mL) was added anhydrous potassium carbonate (26.76 mg, 193.65 μmol) and the mixture was purged by bubbling nitrogen gas through for 5 min. Then Pd(dppf)Cl₂·DCM (158.15 mg, 193.65 μmol) was added and the tube was sealed. The reaction mixture was heated at 80° C. for 16 h. Upon completion, the reaction mixture was cooled to ambient temperature, diluted with ethyl acetate (15 mL) and filtered through Celite. The filtrate was concentrated under reduced pressure and the residue purified by flash silica gel column chromatography (60-80% of Ethyl acetate in petroleum ether) to afford tert-butyl 4-[1-(2,6-dibenzyloxy-3-pyridyl)-3-methyl-2-oxo-benzimidazol-4-yl]-3,6-dihydro-2H-pyridine-1-carboxylate (B-111, 100 mg, 144.33 μmol, 75% yield) as an off white solid. LCMS (ES+): m/z 618.9 [M+H]⁺.

### Step 6: tert-butyl 4-[1-(2,6-dioxo-3-piperidyl)-3-methyl-2-oxo-benzimidazol-4-yl]piperidine-1-carboxylate (B-112)

tert-butyl 4-[1-(2,6-dioxo-3-piperidyl)-3-methyl-2-oxo-benzimidazol-4-yl]piperidine-1-carboxylate (B-112, 80 mg, 170.54 μmol, 95% yield) was synthesized from tert-butyl 4-[1-(2,6-dibenzyloxy-3-pyridyl)-3-methyl-2-oxo-benzimidazol-4-yl]-3,6-dihydro-2H-pyridine-1-carboxylate (B-111) in a similar fashion to Compound B-103, except using 0.1 eq. palladium hydroxide (10% on carbon). LCMS (ES+): m/z 387.3 [M−tBu+H]⁺.

### Step 7: 3-[3-methyl-2-oxo-4-(4-piperidyl)benzimidazol-1-yl]piperidine-2,6-dione (B-113)

3-[3-methyl-2-oxo-4-(4-piperidyl)benzimidazol-1-yl]piperidine-2,6-dione (B-113, 70 mg, 128.02 μmol, 66% yield, TFA salt) was synthesized from tert-butyl4-[1-(2,6-dioxo-3-piperidyl)-3-methyl-2-oxo-benzimidazol-4-yl]piperidine-1-carboxylate (B-112) in a similar fashion to Compound A-62, except using 1 eq. TFA. The material was triturated with diethyl ether. LCMS (ES+): m/z 343.0 [M+H]⁺.

### 3-[1-(2,6-dioxo-3-piperidyl)-3-methyl-2-oxo-benzimidazol-4-yl]propanoic acid (B-116)

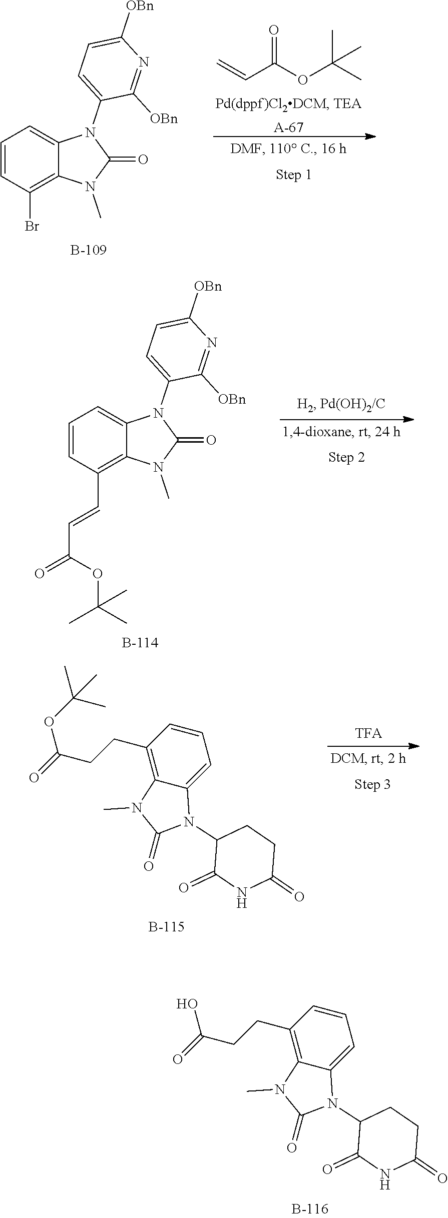

### Step 1: tert-butyl (E)-3-[1-(2,6-dibenzyloxy-3-pyridyl)-3-methyl-2-oxo-benzimidazol-4-yl]prop-2-enoate (B-114)

tert-butyl (E)-3-[1-(2,6-dibenzyloxy-3-pyridyl)-3-methyl-2-oxo-benzimidazol-4-yl]prop-2-enoate (B-114, 80 mg, 136.26 μmol, 70% yield) was synthesized from 4-bromo-1-(2,6-dibenzyloxy-3-pyridyl)-3-methyl-benzimidazol-2-one (B-109) and tert-butyl prop-2-enoate (A-67) in a similar fashion to Compound B-93, except using 5 eq. A-67 and 5 eq. triethylamine. The reaction was heated at 110° C. for 16 h. Upon completion, the reaction was filtered through Celite, washing with ethyl acetate. The filtrate was washed with water (2×), dried over anhydrous sodium sulfate and filtered. The solvent was removed under reduced pressure and the residue purified by silica gel column chromatography (5-10% of Ethyl acetate in petroleum ether). LCMS (ES+): m/z 564.3 [M+H]⁺.

### Step 2: tert-butyl 3-[1-(2,6-dioxo-3-piperidyl)-3-methyl-2-oxo-benzimidazol-4-yl]propanoate (B-115)

tert-butyl 3-[1-(2,6-dioxo-3-piperidyl)-3-methyl-2-oxo-benzimidazol-4-yl]propanoate (B-115, 80 mg, 172.82 μmol, 97% yield) was synthesized from tert-butyl (E)-3-[1-(2,6-dibenzyloxy-3-pyridyl)-3-methyl-2-oxo-benzimidazol-4-yl]prop-2-enoate (B-114) in a similar fashion to Compound B-103, except using 0.3 eq. palladium hydroxide (10% on carbon) and washing the Celite bed with 1,4-dioxane. LCMS (ES+): m/z 331.9 [M−tBu+H]⁺.

### Step 3: 3-[1-(2,6-dioxo-3-piperidyl)-3-methyl-2-oxo-benzimidazol-4-yl]propanoic acid (B-116)

3-[1-(2,6-dioxo-3-piperidyl)-3-methyl-2-oxo-benzimidazol-4-yl]propanoic acid (B-116, 70 mg, 123.08 μmol, 60% yield) was synthesized from tert-butyl 3-[1-(2,6-dioxo-3-piperidyl)-3-methyl-2-oxo-benzimidazol-4-yl]propanoate (B-115) in a similar fashion to Compound A-26, except using 44 eq. TFA. LCMS (ES+): m/z 331.9 [M+H]⁺.

1-[2-[[2-(2,6-dioxo-3-piperidyl)-1,3-dioxo-isoindolin-4-yl]oxyacetyl]piperidine-4-carboxylic acid (B-117)

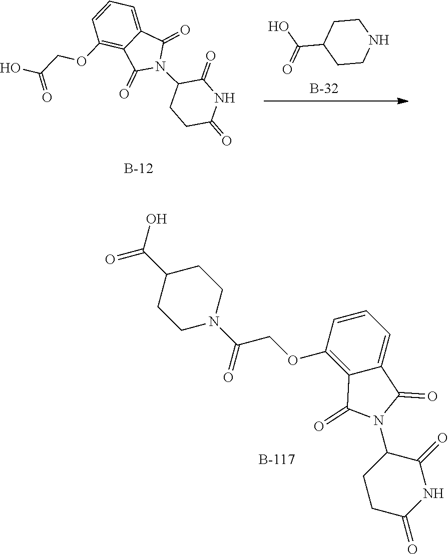

### Step 1: 1-[2-[[2-(2,6-dioxo-3-piperidyl)-1,3-dioxo-isoindolin-4-yl]oxyacetyl]piperidine-4-carboxylic acid (B-117)

Into a 25 mL single neck round bottom flask containing a well-stirred solution of 2-[[2-(2,6-dioxo-3-piperidyl)-1,3-dioxo-isoindolin-4-yl]oxyacetic acid (B-12, 300 mg, 902.90 μmol) in a mixture of anhydrous THF (5 mL) and anhydrous DMF (0.5 mL) was added 1,1′-carbonyldiimidazole (CDI) (366.01 mg, 2.26 mmol). After 4 h a solution of piperidine-4-carboxylic acid (B-32, 116.61 mg, 902.90 μmol, TFA salt) in anhydrous DMF (2 mL) was added. The reaction mixture was stirred for 12 h. The volatiles were removed under reduced pressure and the residue purified using reverse phase prep HPLC [Method: Column-SunFire C18 (150×19) mm, 5 μm; Mobile phase A: 0.1% TFA in water and Mobile phase B: MeCN] to obtain 1-[2-[[2-(2,6-dioxo-3-piperidyl)-1,3-dioxo-isoindolin-4-yl]oxyacetyl]piperidine-4-carboxylic acid (B-117, 180 mg, 395.39 μmol, 44% yield, TFA salt) as an off-white solid. LCMS (ES+): m/z 444.2 [M+H]⁺.

### 3-[1-oxo-4-(4-piperidylamino)isoindolin-2-yl]piperidine-2,6-dione (B-119)

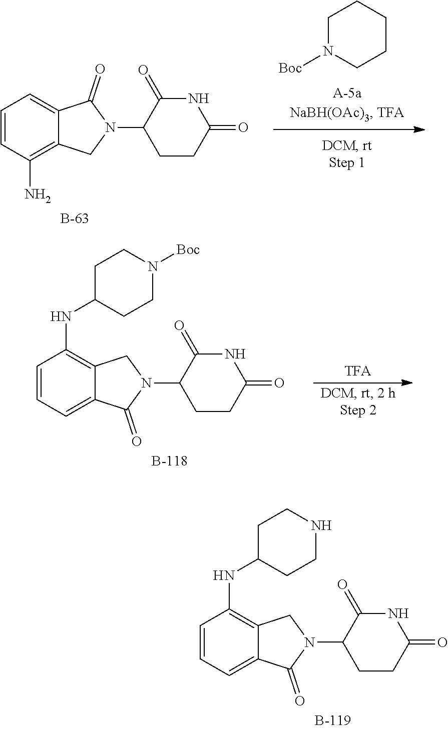

### Step 1: tert-butyl 4-[[2-(2,6-dioxo-3-piperidyl)-1-oxo-isoindolin-4-yl]amino]piperidine-1-carboxylate (B-118)

Into a 10 mL single neck round bottom flask containing a well-stirred solution of 3-(4-amino-1-oxo-isoindolin-2-yl)piperidine-2,6-dione (B-63, 2.0 g, 7.71 mmol) and tert-butyl 4-oxopiperidine-1-carboxylate (A-5a, 2.31 g, 11.57 mmol) in DCM (30 mL) was added TFA (879.60 mg, 7.71 mmol, 594.33 μL) dropwise at 0° C. and the reaction mixture was stirred for 10 min at 0° C. Then, sodium triacetoxy borohydride (4.09 g, 19.29 mmol) was added and the mixture was stirred at room temperature for 16 h. The reaction mixture was diluted with water (30 mL) and the organic layer was extracted with DCM (3×30 mL). The combined organic layer was dried over sodium sulfate, filtered and concentrated under reduced pressure. The residue was triturated with acetone (30 mL), filtered and dried to afford tert-butyl 4-[[2-(2,6-dioxo-3-piperidyl)-1-oxo-isoindolin-4-yl]amino]piperidine-1-carboxylate (B-118, 3.0 g, 6.66 mmol, 87% yield) as an off-white solid, which was used for the next step without further purification. LCMS (ES+): m/z 386.9 [M−tBu+H]⁺.

### Step 2: 3-[1-oxo-4-(4-piperidylamino)isoindolin-2-yl]piperidine-2,6-dione (B-119)

3-[1-oxo-4-(4-piperidylamino)isoindolin-2-yl]piperidine-2,6-dione (B-119, 3.0 g, 5.86 mmol, 28% yield, TFA salt) was synthesized from tert-butyl 4-[[2-(2,6-dioxo-3-piperidyl)-1-oxo-isoindolin-4-yl]amino]piperidine-1-carboxylate (B-118) in a similar fashion to Compound A-62, except using 4 eq. TFA. LCMS (ES+): m/z 343.3 [M+H]⁺.

### 3-(1-oxo-4-(3-oxo-3-(piperazin-1-yl) propyl)isoindolin-2-yl)piperidine-2,6-dione (B-126)

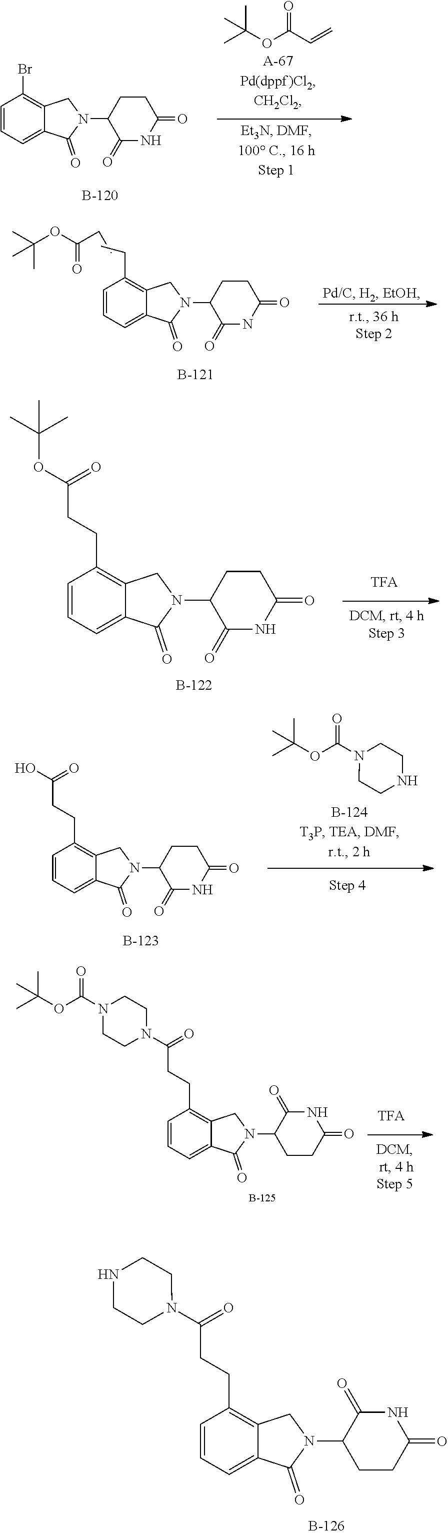

### Step 1: tert-butyl 3-(2-(2,6-dioxopiperidin-3-yl)-1-oxoisoindolin-4-yl)acrylate (B-121)

tert-butyl 3-(2-(2,6-dioxopiperidin-3-yl)-1-oxoisoindolin-4-yl)acrylate (B-121, 2.5 g, 6.48 mmol, 70% yield) was synthesized from 3-(4-bromo-1-oxo-isoindolin-2-yl)piperidine-2,6-dione (B-120) and tert-butyl prop-2-enoate (A-67) in a similar fashion to Compound B-93, except using 3 eq. A-67. Upon completion, the mixture was filtered through Celite, solvent removed under reduced pressure and the residue purified by flash silica gel (230-400 mesh) column chromatography (80% Ethyl acetate in petroleum ether). LCMS (ES+): m/z 371.2 [M+H]⁺.

### Step 2: tert-butyl 3-(2-(2,6-dioxopiperidin-3-yl)-1-oxoisoindolin-4-yl))propanoate (B-122)

tert-butyl 3-(2-(2,6-dioxopiperidin-3-yl)-1-oxoisoindolin-4-yl)propanoate (B-122, 2.1 g, 4.73 mmol, 70% yield) was synthesized from tert-butyl 3-[[2-(2,6-dioxo-3-piperidyl)-1-oxo-isoindolin-4-yl]prop-2-enoate (B-121) in a similar fashion to Compound B-6, except using 0.27 eq. palladium (10% on carbon, wet) under hydrogen bladder. The material was used in the next step without further purification. LCMS (ES+): m/z 373.1 [M+H]⁺.

### Step 3: 3-(2-(2,6-dioxopiperidin-3-yl)-1-oxoisoindolin-4-yl)propanoic acid (B-123)

3-(2-(2,6-dioxopiperidin-3-yl)-1-oxoisoindolin-4-yl)propanoic acid (B-123, 1.65 g, 4.96 mmol, 88% yield) was synthesized from tert-butyl 3-[[2-(2,6-dioxo-3-piperidyl)-1-oxo-isoindolin-4-yl]propanoate (B-122) in a similar fashion to Compound A-26, except using 46 eq. TFA. LCMS (ES+): m/z 317.0 [M+H]⁺.

### Step 4: tert-butyl 4-(3-(2-(2,6-dioxopiperidin-3-yl)-1-oxoisoindolin-4-yl)propanoyl) piperazine-1-carboxylate (B-125)

tert-butyl 4-(3-(2-(2,6-dioxopiperidin-3-yl)-1-oxoisoindolin-4-yl)propanoyl) piperazine-1-carboxy late (B-125, 130 mg, 250.59 μmol, 40% yield) was synthesized from tert-butyl piperazine-1-carboxylate (B-124) and 3-(2-(2,6-dioxopiperidin-3-yl)-1-oxoisoindolin-4-yl)propanoic acid (B-123) in a similar fashion to Compound B-10, except using 1.5 eq. B-124 and 2 eq. 1-propanephosphonic anhydride solution (50% in Ethyl acetate). LCMS (ES+): m/z 483.2 [M−H]⁻.

### Step 5: 3-(1-oxo-4-(3-oxo-3-(piperazin-1-yl) propyl)isoindolin-2-yl)piperidine-2,6-dione (B-126)

3-(1-oxo-4-(3-oxo-3-(piperazin-1-yl) propyl)isoindolin-2-yl)piperidine-2,6-dione (B-126, 100 mg, 157.49 μmol, 59% yield, TFA salt) was synthesized from tert-butyl 4-[3-[[2-(2,6-dioxo-3-piperidyl)-1-oxo-isoindolin-4-yl]propanoyl]piperazine-1-carboxylate (B-125) in a similar fashion to Compound A-62, except using 48 eq. TFA. LCMS (ES+): m/z 385.2 [M+H]⁺.

### 1-(3-(2-(2,6-dioxopiperidin-3-yl)-1-oxoisoindolin-4-yl)propanoyl)piperidine-4-carboxylic acid (B-129)

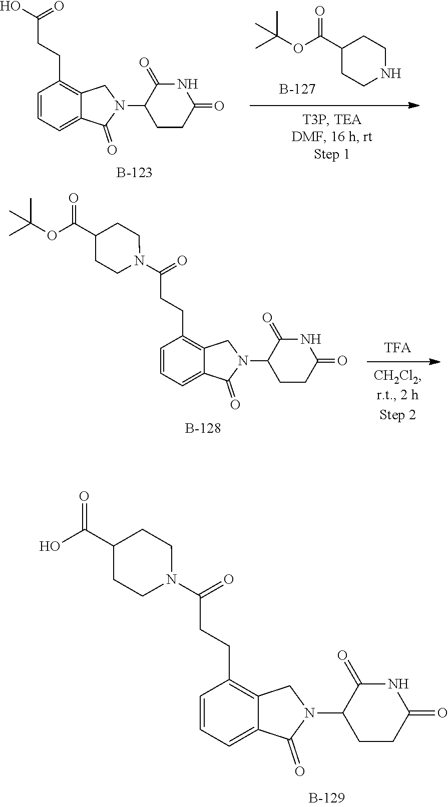 [SMILES: O=C(O)CCc1cccc2c1CN(C1CCC(=O)NC1=O)C2=O ; CC(C)(C)OC(=O)C1CCNCC1 ; CC(C)(C)OC(=O)C1CCN(C(=O)CCc2cccc3c2CN(C2CCC(=O)NC2=O)C3=O)CC1 ; O=C1CCC(N2Cc3c(CCC(=O)N4CCC(C(=O)O)CC4)cccc3C2=O)C(=O)N1]

### Step 1: tert-butyl 1-(3-(2-(2,6-dioxopiperidin-3-yl)-1-oxoisoindolin-4-yl)propanoyl)piperidine-4-carboxylate (B-128)

tert-butyl 1-(3-(2-(2,6-dioxopiperidin-3-yl)-1-oxoisoindolin-4-yl)propanoyl)piperidine-4-carboxylate (B-128, 50 mg, 95.49 μmol, 30% yield) was synthesized from tert-butyl piperidine-4-carboxy late (B-127) and 3-(2-(2,6-dioxopiperidin-3-yl)-1-oxoisoindolin-4-yl)propanoic acid (B-123) in a similar fashion to Compound B-10, except using 1.5 eq. B-127 and 2 eq. 1-propanephosphonic anhydride solution (50% in Ethyl acetate). LCMS (ES+): m/z 482.2 [M−H]⁻.

### Step 2:1-(3-(2-(2,6-dioxopiperidin-3-yl)-1-oxoisoindolin-4-yl)propanoyl)piperidine-4-carboxylic acid (B-129)

1-(3-(2-(2,6-dioxopiperidin-3-yl)-1-oxoisoindolin-4-yl)propanoyl)piperidine-4-carboxylic acid (B-129, 40 mg, 87.60 μmol, 85% yield) was synthesized from tert-butyl 1-[3-[[2-(2,6-dioxo-3-piperidyl)-1-oxo-isoindolin-4-yl]propanoyl]piperidine-4-carboxylate (B-128) in a similar fashion to Compound A-26, except using 25 eq. TFA. LCMS (ES+): m/z 426.2 [M−H]⁻.

### 3-(1-oxo-4-((2-oxo-2-(piperazin-1-yl)ethyl)amino)isoindolin-2-yl)piperidine-2,6-dione (B-131)

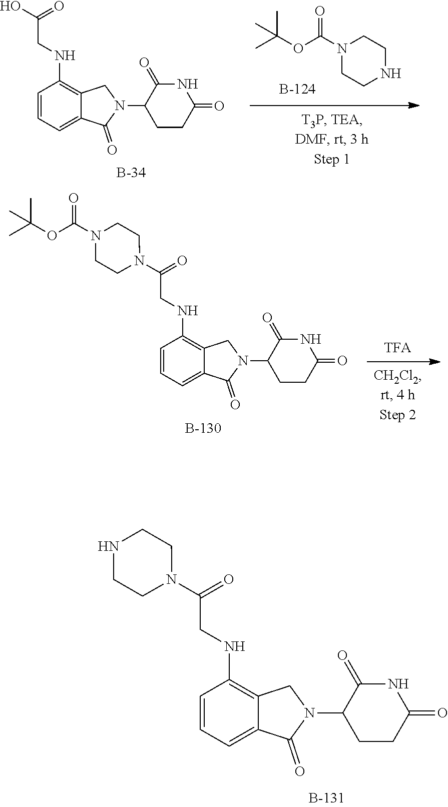

### Step 1: tert-butyl 4-((2-(2,6-dioxopiperidin-3-yl)-1-oxoisoindolin-4-yl)glycyl)piperazine-1-carboxylate (B-130)

tert-butyl 4-((2-(2,6-dioxopiperidin-3-yl)-1-oxoisoindolin-4-yl)glycyl)piperazine-1-carboxylate (B-130, 250 mg, 378.97 μmol, 35% yield) was synthesized from ter-butyl piperazine-1-carboxylate (B-124) and 2-[[2-(2,6-dioxo-3-piperidyl)-1-oxo-isoindolin-4-yl]amino]acetic acid (B-34) in a similar fashion to Compound B-10, except using 0.8 eq. B-124 and 1.5 eq. 1-propanephosphonic anhydride solution (50% in Ethyl acetate). LCMS (ES−): m/z 484.3 [M−H]⁻.

### Step 2: 3-(1-oxo-4-((2-oxo-2-(piperazin-1-yl)ethyl)amino)isoindolin-2-yl)piperidine-2,6-dione (B-131)

3-(1-oxo-4-((2-oxo-2-(piperazin-1-yl)ethyl)amino)isoindolin-2-yl)piperidine-2,6-dione (B-131, 200 mg, 272.31 μmol, 53% yield, TFA salt) was synthesized from tert-butyl 4-[2-[[2-(2,6-dioxo-3-piperidyl)-1-oxo-isoindolin-4-yl]amino]acetyl]piperazine-1-carboxylate (B-130) in a similar fashion to Compound A-62, except using 13 eq. TFA. LCMS (ES+): m/z 386.2 [M+H]⁺.

### 2-[4-[1-(2,6-dioxo-3-piperidyl)-3-methyl-2-oxo-benzimidazol-5-yl]-1-piperidyl]acetic acid (B-137)

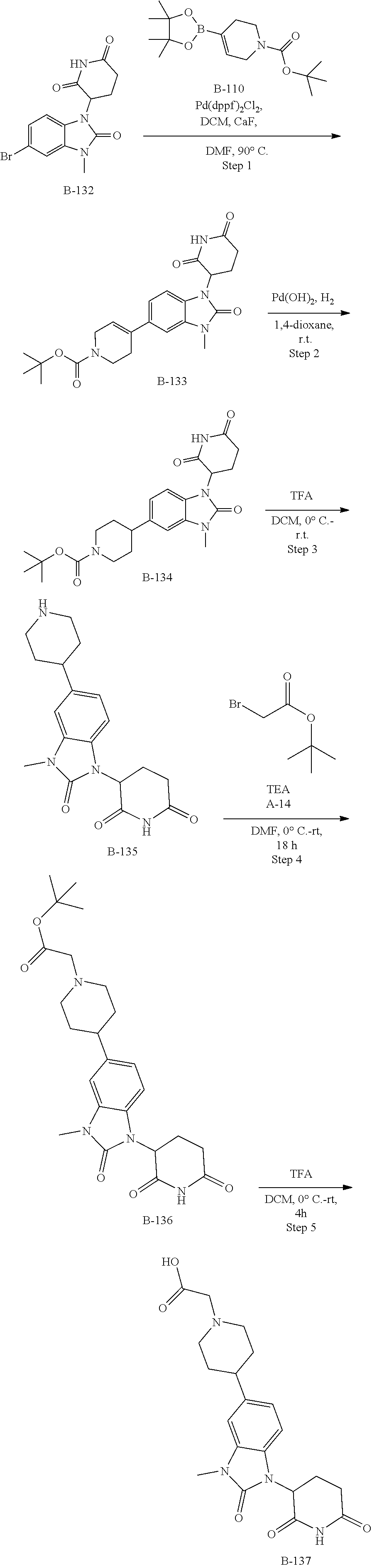

### Step 1: tert-butyl 4-[1-(2,6-dioxo-3-piperidyl)-3-methyl-2-oxo-benzimidazol-S-yl]-3,6-dihydro-2H-pyridine-1-carboxylate (B-133)

Into a 50 mL sealed tube reactor containing a well-stirred solution of 3-(5-bromo-3-methyl-2-oxo-benzimidazol-1-yl)piperidine-2,6-dione (B-132, 1 g, 2.96 mmol) and tert-butyl 4-(4,4,5,5-tetramethyl-1,3,2-dioxaborolan-2-yl)-3,6-dihydro-2H-pyridine-1-carboxylate (B-110, 1.19 g, 3.84 mmol) in anhydrous DMF (8 mL) was added CsF (1.35 g, 8.87 mmol) under nitrogen atmosphere and the resulting mixture was purged by bubbling nitrogen gas into the reaction mixture for 10 min. Subsequently, [1,1′-Bis(diphenylphosphino)ferrocene]dichloropalladium(II) complex with dichloromethane (143.74 mg, 0.176 mmol) was added and the reaction mixture was heated at 90° C. for 16 h. The reaction mixture was poured into water (50 mL) and extracted with Ethyl acetate (2×150 mL). Organic phases were combined and washed with brine (100 mL), dried over sodium sulfate, filtered and concentrated under reduced pressure. The residue was purified by flash silica-gel (230-400 mesh) column, eluting with 0-100% Ethyl acetate/petroleum ether to afford tert-butyl 4-[1-(2,6-dioxo-3-piperidyl)-3-methyl-2-oxo-benzimidazol-5-yl]-3,6-dihydro-2H-pyridine-1-carboxylate (B-133, 0.7 g, 1.49 mmol, 50% yield) as an off-white solid. LCMS (ESI): m/z 441.2 [M+H]⁺.

### Step 2: tert-butyl 4-[1-(2,6-dioxo-3-piperidyl)-3-methyl-2-oxo-benzimidazol-5-yl]piperidine-1-carboxylate (B-134)

tert-butyl 4-[1-(2,6-dioxo-3-piperidyl)-3-methyl-2-oxo-benzimidazol-5-yl]piperidine-1-carboxylate (B-134, 0.35 g, 0.696 mmol, 88% yield) was synthesized from tert-butyl 4-[1-(2,6-dioxo-3-piperidyl)-3-methyl-2-oxo-benzimidazol-5-yl]-3,6-dihydro-2H-pyridine-1-carboxylate (B-133) in a similar fashion to Compound B-103, except using 0.15 eq. palladium hydroxide on carbon (20% by weight, 50% water). The Celite bed was washed with dioxane and 1:1 Ethyl acetate/DCM. Following removal of solvent, the material was triturated with diethyl ether. LCMS (ESI): m/z 387 [M−tBu+H]⁺.

### Step 3: 3-[3-methyl-2-oxo-5-(4-piperidyl)benzimidazol-1-yl]piperidine-2,6-dione (B-135)

3-[3-methyl-2-oxo-5-(4-piperidyl)benzimidazol-1-yl]piperidine-2,6-dione (B-135, 500 mg, 1.28 mmol, 81% yield, TFA salt) was synthesized from tert-butyl 4-[1-(2,6-dioxo-3-piperidyl)-3-methyl-2-oxo-benzimidazol-5-yl]piperidine-1-carboxy late (B-134) in a similar fashion to Compound A-62, except using 15 eq. TFA. The material was triturated with diethyl ether. LCMS (ESI): m/z 343.0 [M+H]⁺.

### Step 4: tert-butyl 2-[4-[1-(2,6-dioxo-3-piperidyl)-3-methyl-2-oxo-benzimidazol-5-yl]-1-piperidyl]acetate (B-136)

tert-butyl 2-[4-[1-(2,6-dioxo-3-piperidyl)-3-methyl-2-oxo-benzimidazol-5-yl]-1-piperidyl]acetate (B-136, 235 mg, 502.39 μmol, 83% yield) was synthesized from 3-[3-methyl-2-oxo-5-(4-piperidyl)benzimidazol-1-yl]piperidine-2,6-dione (B-135) and tert-butyl 2-bromoacetate (A-14) in a similar fashion to Compound B-82, except using 1.3 eq. A-14 and 5 eq. triethylamine. Upon completion, the reaction was diluted with water (35 mL) and the precipitate was filtered and washed with water. The solid was dried under vacuum and used in the next step without further purification. LCMS (ES+): m/z 457.2 [M+H]⁺.

### Step 5: 2-[4-[1-(2,6-dioxo-3-piperidyl)-3-methyl-2-oxo-benzimidazol-5-yl]-1-piperidyl]acetic acid (B-137)

2-[4-[1-(2,6-dioxo-3-piperidyl)-3-methyl-2-oxo-benzimidazol-5-yl]-1-piperidyl]acetic acid (B-137, 200 mg, 349.89 μmol, 71% yield, TFA salt) was synthesized from tert-butyl 2-[4-[1-(2,6-dioxo-3-piperidyl)-3-methyl-2-oxo-benzimidazol-5-yl]-1-piperidyl]acetate (B-136) in a similar fashion to Compound A-26, except using 20 eq. TFA. LCMS (ES+): m/z 400.1 [M+H]⁺.

### 2-[4-[3-(2,6-dioxo-3-piperidyl)-2-oxo-1,3-benzoxazol-6-yl]-1-piperidyl]acetic acid (B-145)

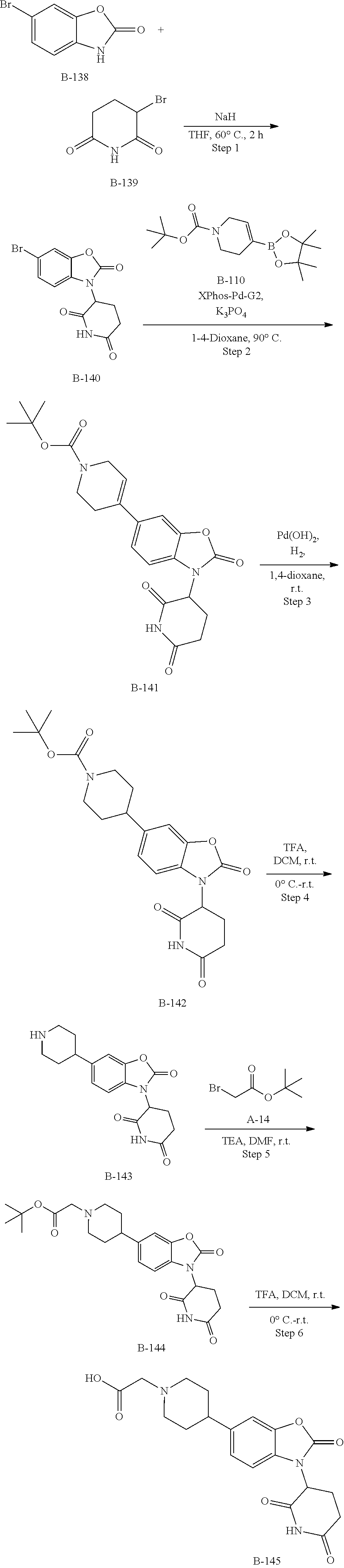

### Step 1: 3-(6-bromo-2-oxobenzo[d]oxazol-3 (2H)-yl)piperidine-2,6-dione (B-140)

To a stirred solution of 6-bromo-3H-1,3-benzoxazol-2-one (B-138, 6 g, 28.04 mmol) in THF (200 mL) was added sodium hydride (60% dispersion in mineral oil) (1.29 g, 56.07 mmol) portion wise and the mixture was heated at 60° C. for 1 h. This mixture was added dropwise via cannula to a stirred solution of 3-bromopiperidine-2,6-dione (B-139, 8.07 g, 42.05 mmol) in THF (50 mL) at 60° C. and stirred for 2 h. The reaction was quenched with saturated ammonium chloride solution and extracted with ethyl acetate. The combined organic layer was washed with water and brine, dried over anhydrous sodium sulfate, filtered and concentrated. The residue was purified by silica gel chromatography (50% Ethyl acetate:Hexanes) to afford 3-(6-bromo-2-oxobenzo[d]oxazol-3 (2H)-yl)piperidine-2,6-dione (B-140, 2.9 g, 8.71 mmol, 31% yield). LCMS (ES−): m/z 323.0 [M−H]⁻.

¹H NMR (400 MHz, DMSO-D6)/11.23 (s, 1H), 7.73 (s, 1H), 7.43 (d, J=8.4 Hz, 1H), 7.26 (d, J=8.36 Hz, 1H), 5.41-5.37 (m, 1H), 2.87-2.84 (m, 1H), 2.71-2.64 (m, 2H), 2.18-2.15 (m, 1H)

### Step 2: tert-butyl 4-[3-(2,6-dioxo-3-piperidyl)-2-oxo-1,3-benzoxazol-6-yl]-3,6-dihydro-2H-pyridine-1-carboxylate (B-141)

Into a 20 mL sealed tube containing a well-stirred solution of 3-(6-bromo-2-oxo-1,3-benzoxazol-3-yl)piperidine-2,6-dione (B-140, 200 mg, 0,615 mmol) and tert-butyl 4-(4,4,5,5-tetramethyl-1,3,2-dioxaborolan-2-yl)-3,6-dihydro-2H-pyridine-1-carboxylate (B-110, 247.28 mg, 0.800 mmol) in 1,4-dioxane (2 mL) was added anhydrous potassium phosphate tribasic (522.32 mg, 2.46 mmol) under nitrogen atmosphere and the resulting mixture was degassed by bubbling nitrogen gas through the reaction mixture for 10 min. Finally, XPhos-Pd-G2 (48.40 mg, 0.0615 mmol) was added to the reaction mixture and the reaction was heated to 90° C. for 16 h. The reaction was cooled to room temperature and the reaction mixture was poured into water (10 mL) and extracted with Ethyl acetate (2×10 mL). The combined organic layer was washed with brine solution (10 mL), dried over sodium sulfate, filtered and concentrated under reduced pressure. The residue was purified by silica-gel (230-400 mesh) flash column, eluting with 0-100% Ethyl acetate/petroleum ether to afford tert-butyl 4-[3-(2,6-dioxo-3-piperidyl)-2-oxo-1,3-benzoxazol-6-yl]-3,6-dihydro-2H-pyridine-1-carboxylate (B-141, 200 mg, 0.429 mmol, 70% yield) as an off-white solid. LCMS (ESI): m/z 426.2 [M−H]⁻.

### Step 3: tert-butyl 4-[3-(2,6-dioxo-3-piperidyl)-2-oxo-1,3-benzoxazol-6-yl]piperidine-1-carboxylate (B-142)

tert-butyl 4-[3-(2,6-dioxo-3-piperidyl)-2-oxo-1,3-benzoxazol-6-yl]piperidine-1-carboxylate (B-142, 190 mg, 0.384 mmol, 82% yield) was synthesized from tert-butyl 4-[3-(2,6-dioxo-3-piperidyl)-2-oxo-1,3-benzoxazol-6-yl]-3,6-dihydro-2H-pyridine-1-carboxylate (B-141) in a similar fashion to Compound B-103, except using 0.3 eq. palladium hydroxide on carbon (20% by weight, 50% water). The Celite pad was washed with 1:1 Ethyl acetate/DCM, and the material was triturated with diethyl ether. LCMS (ESI): m/z 427.9 [M−H]⁻.

### Step 4: 3-[2-oxo-6-(4-piperidyl)-1,3-benzoxazol-3-yl]piperidine-2,6-dione (B-143)

3-[2-oxo-6-(4-piperidyl)-1,3-benzoxazol-3-yl]piperidine-2,6-dione (B-143, 170 mg, 0.303 mmol, 47% yield, TFA salt) was synthesized from tert-butyl 4-[3-(2,6-dioxo-3-piperidyl)-2-oxo-1,3-benzoxazol-6-yl]piperidine-1-carboxylate (B-142) in a similar fashion to Compound A-62, except using 45 eq. TFA. The material was triturated with diethyl ether. LCMS (ESI): m/z 330.2 [M+H]⁺.

### Step 5: tert-butyl 2-[4-[3-(2,6-dioxo-3-piperidyl)-2-oxo-1,3-benzoxazol-6-yl]-1-piperidyl]acetate (B-144)

tert-butyl 2-[4-[3-(2,6-dioxo-3-piperidyl)-2-oxo-1,3-benzoxazol-6-yl]-1-piperidyl]acetate (B-144, 150 mg, 0.305 mmol, 67% yield) was synthesized from 3-[2-oxo-6-(4-piperidyl)-1,3-benzoxazol-3-yl]piperidine-2,6-dione (B-143) and tert-butyl 2-bromoacetate (A-14) in a similar fashion to Compound B-82, except using 1.1 eq. A-14 and 5 eq. triethylamine. Upon completion, the reaction mixture was poured into ice cold water and the precipitate filtered and dried under reduced pressure. The material was used in the next step without further purification. LCMS (ESI): m/z 444.0 [M+H]⁺.

### Step 6: 2-[4-[3-(2,6-dioxo-3-piperidyl)-2-oxo-1,3-benzoxazol-6-yl]-1-piperidyl]acetic acid (B-145)

2-[4-[3-(2,6-dioxo-3-piperidyl)-2-oxo-1,3-benzoxazol-6-yl]-1-piperidyl]acetic acid (B-145, 150 mg, 0.234 mmol, 55% yield) was synthesized from tert-butyl 2-[4-[3-(2,6-dioxo-3-piperidyl)-2-oxo-1,3-benzoxazol-6-yl]-1-piperidyl]acetate (B-144) in a similar fashion to Compound A-26, except using 45 eq. TFA. The material was azeotroped with toluene (2×20 mL) and subsequently triturated with diethyl ether (2×10 mL). LCMS (ESI): m/z 387.9 [M+H]⁺.

### 3-(4-((2-(2,6-dioxopiperidin-3-yl)-1-oxoisoindolin-4-yl)oxy)piperidin-1-yl)propanoic acid (B-150)

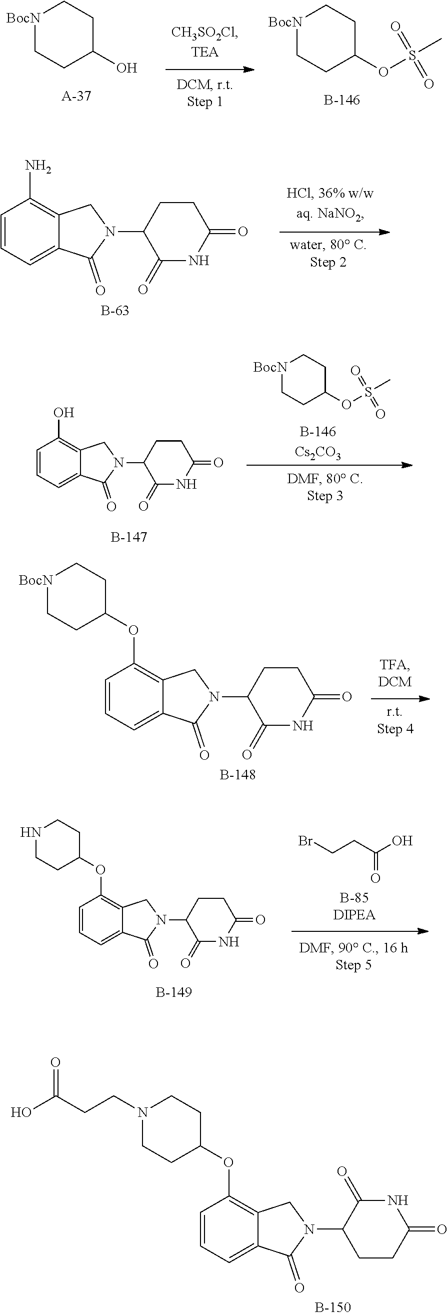

### Step 1: tert-butyl 4-methylsulfonyloxypiperidine-1-carboxylate (B-146)

Into a 100 mL single neck round bottom flask containing a well-stirred solution of tert-butyl 4-hydroxypiperidine-1-carboxylate (A-37, 3.0 g, 14.91 mmol) in anhydrous DCM (30 mL) were added triethylamine (3.77 g, 37.26 mmol, 5.19 mL) and methanesulfonyl chloride (2.56 g, 22.36 mmol, 1.73 mL) at 0° C. under nitrogen atmosphere. The resulting mixture was stirred at room temperature for 3 h. The reaction mixture was diluted with water (2×50 mL) and extracted with DCM (2×30 mL). The combined organic layers were dried over sodium sulfate, filtered and concentrated under reduced pressure to afford tert-butyl 4-methylsulfonyloxypiperidine-1-carboxylate (B-146, 2.5 g, 72% yield) as an off-white solid. The material was used in the next step without further purification. GCMS (ESI): m/z 279.1 [M]⁺. ¹H NMR (400 MHz, CDCl₃). δ 4.92-4.88 (m, 1H), 3.75-3.69 (m, 2H), 3.35-3.29 (m, 2H), 3.05 (s, 3H), 2.00-1.95 (m, 2H), 1.87-1.81 (m, 2H) and 1.47 (s, 9H).

### Step 2: 3-(4-hydroxy-1-oxo-isoindolin-2-yl)piperidine-2,6-dione (B-147)

Into a 250 mL single neck round bottom flask containing a well-stirred solution of 3-(4-amino-1-oxo-isoindolin-2-yl)piperidine-2,6-dione (B-63, 5 g, 19.29 mmol) in water (70 mL) was added sodium nitrite (2.00 g, 28.93 mmol, 919.78 μL) in water (5 mL) at 0° C. The reaction mixture was stirred at room temperature for 5 min. Subsequently, hydrochloric acid (36% w/w aq. soln.) (6.40 g, 175.53 mmol, 8 mL) was added to the reaction at 0° C. and was stirred at room temperature for 15 min before stirring at 80° C. for 2 h. The solvent was removed under reduced pressure and the residue was purified by reverse phase column chromatography (compound eluted by 35% acetonitrile in water (0.1% TEA in water)) to afford 3-(4-hydroxy-1-oxo-isoindolin-2-yl)piperidine-2,6-dione (B-147, 2.1 g, 6.29 mmol, 33% yield) as a brown solid. LCMS (ES+): m/z 261.1 [M+H]⁺. ¹H NMR (400 MHz, DMSO-d₆) δ 10.99 (s, 1H), 10.13 (s, 1H), 7.36-7.32 (m, 1H), 7.19 (d, J=7.2 Hz, 1H), 7.02 (d, J=7.6 Hz, 1H), 5.10 (dd, J=13.2, 5.2 Hz, 1H), 4.35-4.17 (m, 2H), 2.96-2.87 (m, 1H), 2.67-2.51 (m, 1H), 2.46-2.33 (m, 1H) and 2.03-1.97 (m, 1H).

### Step 3: tert-butyl 4-((2-(2,6-dioxopiperidin-3-yl)-1-oxoisoindolin-4-yl)oxy)piperidine-1-carboxylate (B-148)

Into a 50 mL sealed tube containing a well-stirred solution of 3-(4-hydroxy-1-oxoisoindolin-2-yl)piperidine-2,6-dione (B-147, 700 mg, 2.10 mmol) and tert-butyl 4-((methylsulfonyl)oxy)piperidine-1-carboxylate (B-146, 879.13 mg, 3.15 mmol) in anhydrous DMF (8 mL) was added cesium carbonate (1.37 g, 4.20 mmol). The reaction mixture was stirred at 80° C. for 16 h. The reaction mixture was diluted with water (30 mL) and extracted with Ethyl acetate (2×20 mL). The combined organic layers were dried over anhydrous sodium sulfate, filtered and concentrated under reduced pressure. The residue was purified by silica-gel (60-120 mesh) column chromatography, eluting with 80% Ethyl acetate/petroleum ether to afford tert-butyl 4-((2-(2,6-dioxopiperidin-3-yl)-1-oxoisoindolin-4-yl)oxy)piperidine-1-carboxylate (B-148, 700 mg, 0.751 mmol, 36% yield) as a green gummy liquid. LCMS (ESI): m/z 442.2 [M−H]⁻. ¹H NMR (400 MHz, CDCl₃). δ 8.18 (s, 1H), 7.51-7.42 (m, 2H), 7.05 (d, J=8.0 Hz, 1H), 5.26-5.22 (m, 1H), 4.63-4.61 (m, 1H), 4.46-4.28 (m, 2H), 3.72-3.66 (m, 2H), 3.42-3.37 (m, 2H), 2.42-2.38 (m, 1H), 2.26-2.24 (m, 1H), 2.00-1.95 (m, 2H), 1.85-1.77 (m, 4H) and 1.48 (s, 9H).

### Step 4: 3-[1-oxo-4-(4-piperidyloxy)isoindolin-2-yl]piperidine-2,6-dione (B-149)

3-[1-oxo-4-(4-piperidyloxy)isoindolin-2-yl]piperidine-2,6-dione (B-149, 600 mg, 1.06 mmol, 67% yield) was synthesized from tert-butyl 4-((2-(2,6-dioxopiperidin-3-yl)-1-oxoisoindolin-4-yl)oxy)piperidine-1-carboxylate (B-148) in a similar fashion to Compound A-62, except using 41 eq. TFA. LCMS (ES+): m/z 344.1 [M+H]⁺.

### Step 5: 3-(4-((2-(2,6-dioxopiperidin-3-yl)-1-oxoisoindolin-4-yl)oxy)piperidin-1-yl)propanoic acid (B-150)

3-(4-((2-(2,6-dioxopiperidin-3-yl)-1-oxoisoindolin-4-yl)oxy)piperidin-1-yl)propanoic acid (B-150, 100 mg, 229.56 μmol, 23% yield) was synthesized from 3-[1-oxo-4-(4-piperidyloxy)isoindolin-2-yl]piperidine-2,6-dione (B-149) and 3-bromopropanoic acid (B-85) in a similar fashion to Compound B-82, except using 1.1 eq. B-85, and 3 eq. N,N-diisopropylethylamine. Upon completion, the reaction mixture was concentrated under reduced pressure and the residue was purified by reverse phase prep HPLC (Column: XSelect C18 150 mm; Mobile phase: 0.1% TFA in water/MeCN). LCMS (ES+): m/z 416.3 [M+H]⁺.

### 1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]azetidine-3-carboxamide (B-160)

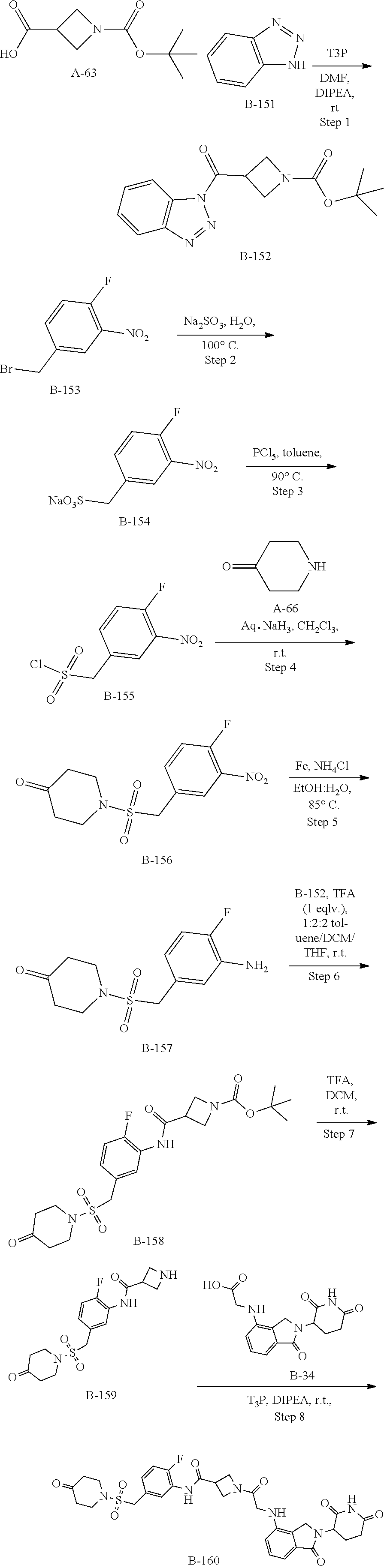

### Step 1: tert-butyl 3-(benzotriazole-2-carbonyl)azetidine-1-carboxylate (B-152)

tert-butyl 3-(benzotriazole-2-carbonyl)azetidine-1-carboxy late (B-152, 1.8 g, 5.36 mmol, 32% yield) was synthesized from 1-tert-butoxycarbonylazetidine-3-carboxylic acid (A-63) and 1H-benzotriazole (B-151) in a similar fashion to Compound A-88, except using 1 eq. B-151, 3 eq. DIPEA and 1.5 eq. 1-propanephosphonic anhydride (50% in ethyl acetate). Upon completion, the reaction was quenched with water and extracted with diethyl ether (3×). The combined organic phases were dried over sodium sulfate, filtered and concentrated under reduced pressure. The residue was purified by flash silica-gel (230-400 mesh) column, eluting with 30% Ethyl acetate/petroleum ether. ¹H NMR (400 MHz, CDCl₃) δ 8.32 (d, J=8.2 Hz, 1H), 8.15 (d, J=8.2 Hz, 1H), 7.73-7.69 (m, 1H), 7.57-7.54 (m, 1H), 4.71-4.63 (m, 1H), 4.39 (d, J=7.5 Hz, 4H) and 1.47 (s, 9H).

### Step 2: sodium (4-fluoro-3-nitrophenyl)methanesulfonate (B-154)

Into a 500 mL single neck round bottom flask containing a well-stirred solution of 4-(bromomethyl)-1-fluoro-2-nitro-benzene (B-153, 10 g, 42.73 mmol) in water (100 mL) was added anhydrous sodium sulphite (5.92 g, 47.00 mmol, 2.25 mL) and the resulting reaction mixture was stirred for 16 h at 100° C. The solvent was removed under reduced pressure and the residue was co-distilled with toluene (3×75 mL) to afford sodium (4-fluoro-3-nitrophenyl)methanesulfonate (B-154, 13 g) as a crude fine powder. This material was taken to the next step without further purification.

### Step 3: (4-fluoro-3-nitro-phenyl)methanesulfonyl chloride (B-155)

Into a 500 mL single neck round bottom flask containing a well-stirred solution of sodium (4-fluoro-3-nitrophenyl)methanesulfonate (B-154, 13 g, crude) in anhydrous toluene (80 mL) was added phosphorus pentachloride (26.69 g, 128.19 mmol, 16.68 mL). Reaction mixture was stirred at 90° C. for 3 h. The solvent was removed under reduced pressure and the residue was dissolved in DCM (300 mL) and washed with cold water (2×100 mL). Combined organic phases were dried over sodium sulfate, filtered and concentrated under reduced pressure to afford (4-fluoro-3-nitro-phenyl)methanesulfonyl chloride (B-155, 10 g) as a brown-coloured syrupy material. The material was used in the next step without further purification.

### Step 4:1-[(4-fluoro-3-nitro-phenyl)methylsulfonyl]piperidin-4-one (B-156)

1-[(4-fluoro-3-nitro-phenyl)methylsulfonyl]piperidin-4-one (B-156, 2.5 g, 7.11 mmol, 19% yield) was synthesized from piperidin-4-one (A-66) and (4-fluoro-3-nitro-phenyl)methanesulfonyl chloride (B-155) in a similar fashion to Compound A-8, except using 1 eq. B-155. Upon completion, the layers were separated, and the organic layer was dried over sodium sulfate, filtered and concentrated under reduced pressure. The residue was purified by flash silica-gel (230-400 mesh) column eluting with 40% Ethyl acetate/petroleum ether. ¹H NMR (400 MHz, DMSO-d₆) δ 8.27 (dd, J=7.2, 2.4 Hz, 1H), 7.89-7.85 (m, 1H), 7.67-7.62 (m, 1H), 4.71 (s, 2H), 3.48 (t, J=6.4 Hz, 4H) and 2.40 (t, J=6.0 Hz, 4H).

### Step 5:1-[(3-amino-4-fluoro-phenyl)methylsulfonyl]piperidin-4-one (B-157)

1-[(3-amino-4-fluoro-phenyl)methylsulfonyl]piperidin-4-one (B-157, 1 g, 2.61 mmol, 55% yield) was synthesized from 1-[(4-fluoro-3-nitro-phenyl)methylsulfonyl]piperidin-4-one (B-156) in a similar fashion to Compound B-99, except the filtrate was diluted with Ethyl acetate and the organic layer was washed with water. LCMS (ESI): m/z 287.1 [M+H]⁺. ¹H NMR (400 MHz, DMSO-d₆) δ 7.00-6.95 (m, 1H), 6.83 (dd, J=8.7, 1.9 Hz, 1H), 6.57-6.54 (m, 1H), 5.24 (s, 2H), 4.34 (s, 2H), 3.44 (t, J=6.0 Hz, 4H) and 2.37 (t, J=6.0 Hz, 4H).

### Step 6: tert-butyl 3-[[2-fluoro-5-[(4-oxo-1-piperidyl)sulfonylmethyl]phenyl]carbamoyl]azetidine-1-carboxylate (B-158)

Into a 50 mL single neck round bottom flask containing a well-stirred solution of 1-[(3-amino-4-fluoro-phenyl)methylsulfonyl]piperidin-4-one (B-157, 0.5 g, 1.75 mmol) and tert-butyl 3-(benzotriazole-2-carbonyl)azetidine-1-carboxylate (B-152, 580.75 mg, 1.92 mmol) in a mixture of 2:1 anhydrous DCM/toluene (9 mL) was added trifluoroacetic (398.23 mg, 3.49 mmol, 269.08 μL) at room temperature under nitrogen atmosphere. After addition of trifluoroacetic acid, precipitation was observed and THF (6 mL) was added to dissolve the precipitate. The reaction mixture was stirred at room temperature for 24 b. The solvent was removed under reduced pressure to afford tert-butyl 3-[[2-fluoro-5-[(4-oxo-1-piperidyl)sulfonylmethyl]phenyl]carbamoyl]azetidine-1-carboxylate (B-158, 1.1 g, 918.37 μmol, 53% yield, 39% purity) as a black-coloured syrup. The material was taken to the next step without purification. LCMS (ESI): m/z 468.1 [M−H]⁻.

### Step 7: N-[2-fluoro-5-[(4-oxo-1-piperidyl)sulfonylmethyl]phenyl]azetidine-3-carboxamide (B-159)

N-[2-fluoro-5-[(4-oxo-1-piperidyl)sulfonylmethyl]phenyl]azetidine-3-carboxamide (B-159, 0.5 g, 725.23 μmol, 79% yield) was synthesized from tert-butyl 3-[[2-fluoro-5-[(4-oxo-1-piperidyl)sulfonylmethyl]phenyl]carbamoyl]azetidine-1-carboxylate (B-158) in a similar fashion to Compound A-62, except using 28 eq. TFA. The material was triturated with MTBE. LCMS (ESI): m/z 370.1 [M+H]⁺.

### Step 8:1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]azetidine-3-carboxamide (B-160)

1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]azetidine-3-carboxamide (B-160, 50 mg, 46.75 μmol, 13% yield) was synthesized from N-[2-fluoro-5-[(4-oxo-1-piperidyl)sulfonylmethyl]phenyl]azetidine-3-carboxamide (B-159) and 2-[[2-(2,6-dioxo-3-piperidyl)-1-oxo-isoindolin-4-yl]amino]acetic acid (B-34) in a similar fashion to Compound A-88, except using 2 eq. B-34, 5 eq. DIPEA and 3 eq. 1-propanephosphonic anhydride (50% in ethyl acetate). LCMS (ESI): m/z 669.1 [M+H]⁺.

### 1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]azetidine-3-carboxamide (B-168)

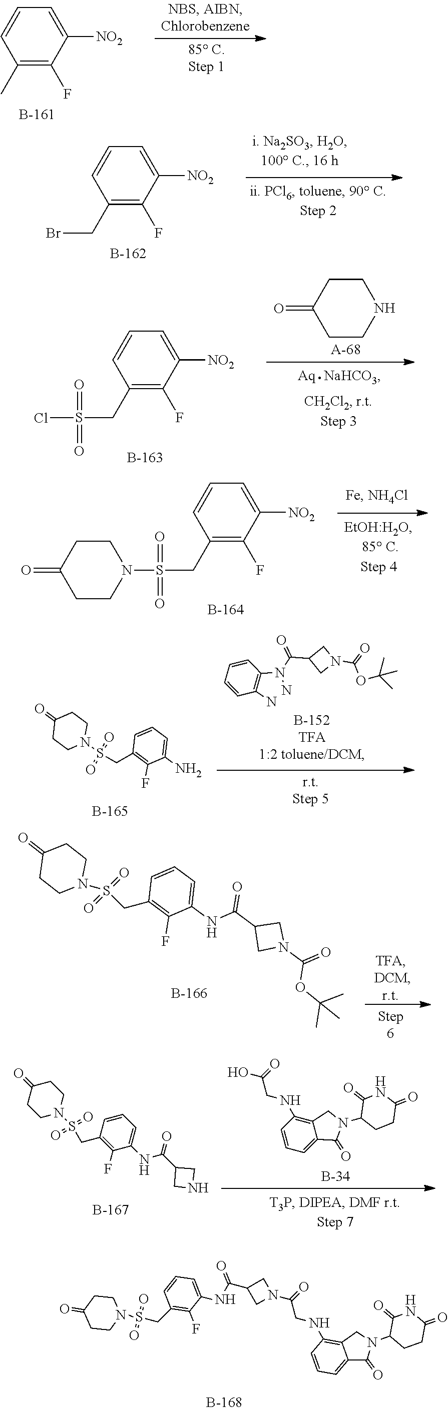

### Step 1: 1-(bromomethyl)-2-fluoro-3-nitro-benzene (B-162)

Into a 1 L three neck round bottom flask containing a well-stirred solution of 2-fluoro-1-methyl-3-nitro-benzene (B-161, 20 g, 128.93 mmol) in anhydrous chlorobenzene (150 mL) were added N-bromosuccinimide (25.24 g, 141.82 mmol, 12.02 mL) and 2-[(E)-(1-cyano-1-methyl-ethyl) azo]-2-methyl-propanenitrile (3.18 g, 19.34 mmol). The reaction mixture was heated to 85° C. for 16 h. The reaction mixture was cooled to room temperature, filtered and the solvent removed. The residue was dissolved in DCM (60 mL) and filtered again. The filtrate was evaporated to dryness under reduced pressure, and the residue was purified by silica-gel (230-400 mesh) flash column chromatography, eluting with 0-100% Ethyl acetate/petroleum ether to afford 1-(bromomethyl)-2-fluoro-3-nitro-benzene (B-162, 18 g, 62.21 mmol, 48% yield) as a yellow-coloured solid. GCMS (ESI): m/z 233.9 [M]⁺. ¹H NMR (400 MHz, DMSO-d₆) δ 8.04-8.00 (m, 1H), 7.74-7.70 (m, 1H), 7.33-7.27 (m, 1H) and 4.56 (s, 2H).

### Step 2: (2-fluoro-3-nitrophenyl)methanesulfonyl chloride (B-163)

Into a 500 mL single neck round bottom flask containing a well-stirred solution of 1-(bromomethyl)-2-fluoro-3-nitro-benzene (B-162, 15 g, 64.10 mmol) in water (150 mL) was added sodium sulphite (8.08 g, 64.10 mmol, 3.07 mL) and the reaction mixture was heated to 100° C. for 16 h. The reaction mixture was concentrated under reduced pressure and the residue was azeotroped with toluene (3×100 mL) and washed with Ethyl acetate (3×100 mL) to afford crude sodium (2-fluoro-3-nitrophenyl)methane sulfonate (18 g) as an off-white solid. About 15 g of this crude was added to a 500 mL single neck round bottom flask in anhydrous toluene (60 mL) and treated with phosphorus pentachloride (26.69 g, 128.19 mmol, 16.68 mL) at room temperature under nitrogen atmosphere. The resulting mixture was stirred at 90° C. for 3 h. The reaction mixture was concentrated under reduced pressure and the residue diluted with water (100 mL) and extracted with DCM (2×100 mL). The combined organic layers were washed with brine (50 mL), dried over sodium sulfate, filtered and concentrated under reduced pressure. The residue was triturated with MTBE (2×50 mL) to afford (2-fluoro-3-nitrophenyl)methanesulfonyl chloride (B-163, 8 g) as a dark-brown liquid which was taken to the next step without further purification.

### Step 3: 1-[(2-fluoro-3-nitro-phenyl)methylsulfonyl]piperidin-4-one (B-164)

1-[(2-fluoro-3-nitro-phenyl)methylsulfonyl]piperidin-4-one (B-164,600 mg, 1.90 mmol, 17% yield) was synthesized from piperidin-4-one hydrochloride (A-66) and (2-fluoro-3-nitrophenyl)methanesulfonyl chloride (B-163) in a similar fashion to Compound A-8, except using 1.5 eq. B-163. Upon completion, water was added and extracted with ethyl acetate. The organic layer was dried over sodium sulfate, filtered and concentrated under reduced pressure. The residue was purified by silica-gel (230-400 mesh) flash column eluting with 0-100% Ethyl acetate/petroleum ether. ¹H NMR (400 MHz, DMSO-d₆) δ 8.18 (t, J=14 Hz, 1H), 7.91 (t, J=12.4 Hz, 1H), 7.50 (t, J=16 Hz, 1H), 4.73 (s, 2H), 3.53 (t, J=12.4 Hz, 4H) and 2.43 (t, J=12.4 Hz, 4H).

### Step 4:1-[(3-amino-2-fluoro-phenyl)methylsulfonyl]piperidin-4-one (B-165)

1-[(3-amino-2-fluoro-phenyl)methylsulfonyl]piperidin-4-one (B-165, 420 mg, 1.47 mmol, 77% yield) was synthesized from 1-[(2-fluoro-3-nitro-phenyl)methylsulfonyl]piperidin-4-one (B-164) in a similar fashion to Compound B-99, except Celite was washed with Ethyl acetate and the organic layer was dried over sodium sulfate, filtered and solvent removed. ¹H NMR (400 MHz, DMSO-d₆) 6.87 (t, J=15.28 Hz, 1H) 6.75 (t, J=16.4 Hz, 1H), 6.59 (t, J=14.4 Hz, 1H), 5.21 (s, 2H), 4.42 (s, 2H), 3.47 (t, J=12.4 Hz, 4H) and 2.41 (m, 4H).

### Step 5: tert-butyl 3-[[2-fluoro-3-[(4-oxo-1-piperidyl)sulfonylmethyl]phenyl]carbamoyl]azetidine-1-carboxylate (B-166)

tert-butyl 3-[[2-fluoro-3-[(4-oxo-1-piperidyl)sulfonylmethyl]phenyl]carbamoyl]azetidine-1-carboxylate (B-166, 300 mg, 0.226 mmol, 43% yield) was synthesized from 1-[(3-amino-2-fluoro-phenyl)methylsulfonyl]piperidin-4-one (B-165) and tert-butyl 3-(benzotriazole-2-carbonyl)azetidine-1-carboxylate (B-152) in a similar fashion to Compound B-158, except using 1 eq. B-152. The material was triturated with diethyl ether. LCMS (ESI): m/z 468.1 [M−H]⁻.

### Step 6: N-[2-fluoro-3-[(4-oxo-1-piperidyl)sulfonylmethyl]phenyl]azetidine-3-carboxamide (B-167)

N-[2-fluoro-3-[(4-oxo-1-piperidyl)sulfonylmethyl]phenyl]azetidine-3-carboxamide (B-167, 190 mg, 0.198 mmol, 31% yield, TFA salt) was synthesized from tert-butyl 3-[[2-fluoro-3-[(4-oxo-1-piperidyl)sulfonylmethyl]phenyl]carbamoyl]azetidine-1-carboxylate (B-166) in a similar fashion to Compound A-62, except using 31 eq. TFA. The material was triturated with diethyl ether. LCMS (ESI): m/z 370.1 [M+H]⁺.

### Step 7:1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]azetidine-3-carboxamide (B-168)

1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]azetidine-3-carboxamide (B-168, 100 mg, 0.134 mmol, 24% yield) was synthesized from 2-[[2-(2,6-dioxo-3-piperidyl)-1-oxo-isoindolin-4-yl]amino]acetic acid (B-34) and N-[2-fluoro-3-[(4-oxo-1-piperidyl)sulfonylmethyl]phenyl]azetidine-3-carboxamide (B-167) in a similar fashion to Compound A-88, except using 1.2 eq. B-34, 4 eq. DIPEA and 1.5 eq. 1-propanephosphonic anhydride (50% in ethyl acetate). LCMS (ESI): m/z 669.1 [M+H]⁺.

### 1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]piperidine-4-carboxamide (B-172)

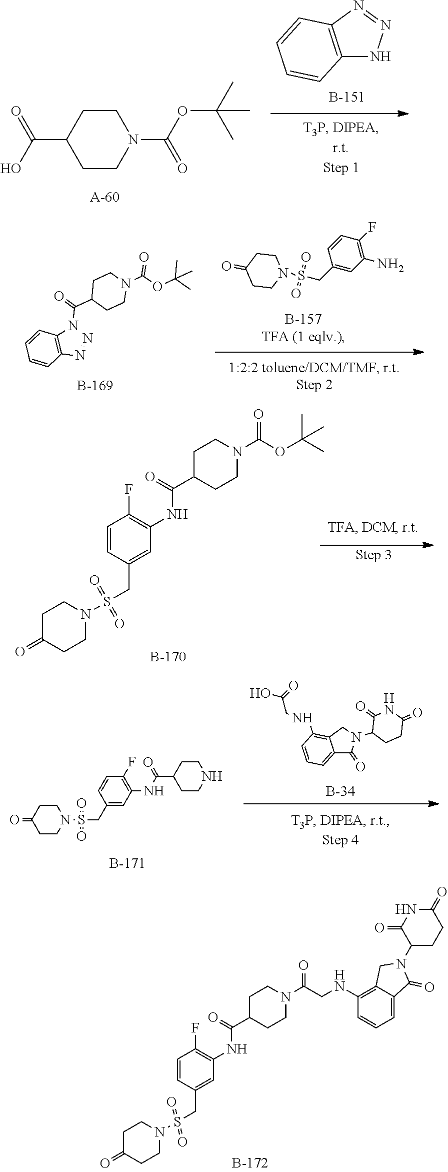

### Step 1: tert-butyl 4-(benzotriazole-1-carbonyl)piperidine-1-carboxylate (B-169)

tert-butyl 4-(benzotriazole-1-carbonyl)piperidine-1-carboxylate (B-169, 600 mg, 1.81 mmol, 83%) was synthesized from 1-tert-butoxycarbonylpiperidine-4-carboxylic acid (A-60) and 1H-benzotriazole (B-151) in a similar fashion to Compound A-88, except using 1 eq. B-151, 3 eq. DIPEA and 1.5 eq. 1-propanephosphonic anhydride (50% in ethyl acetate). Upon completion, the reaction was quenched with cold water and was extracted with Et₂O (2×20 mL). The combined organic layers were dried over sodium sulfate, filtered and concentrated under reduced pressure. The residue was purified by flash silica-gel (230-400 mesh) column with 20% Ethyl acetate/petroleum ether eluent. LCMS (ESI): m/z 331.4 [M+H]⁺. ¹H NMR (400 MHz, DMSO-d₆) δ 8.28-8.23 (m, 2H), 7.82-7.77 (m, 1H), 7.65-7.59 (m, 1H), 4.05-3.98 (m, 3H), 2.98 (br s, 2H), 2.11-2.05 (m, 2H), 1.77-1.66 (m, 2H) and 1.42 (s, 9H).

### Step 2: tert-butyl 4-[[2-fluoro-5-[(4-oxo-1-piperidyl)sulfonylmethyl]phenyl]carbamoyl]piperidine-1-carboxylate (B-170)

tert-butyl 4-[[2-fluoro-5-[(4-oxo-1-piperidyl)sulfonylmethyl]phenyl]carbamoyl]piperidine-1-carboxylate (B-170, 1.3 g, 0.679 mmol, 26% purity) was synthesized from 1-[(3-amino-4-fluoro-phenyl)methylsulfonyl]piperidin-4-one (B-157) and 4-(benzotriazole-1-carbonyl)piperidine-1-carboxy late (B-169) in a similar fashion to Compound B-158. LCMS (ESI): m/z 496.2 [M−H]⁻

### Step 3: N-[2-fluoro-5-[(4-oxo-1-piperidyl)sulfonylmethyl]phenyl]piperidine-4-carboxamide (B-171)

N-[2-fluoro-5-[(4-oxo-1-piperidyl)sulfonylmethyl]phenyl]piperidine-4-carboxamide (B-171, 0.5 g, 0.523 mmol, 76.98% yield, 53% purity) was synthesized from tert-butyl 4-[[2-fluoro-5-[(4-oxo-1-piperidyl)sulfonylmethyl]phenyl]carbamoyl]piperidine-1-carboxylate (B-170) in a similar fashion to Compound A-62, except using 38 eq. TFA. The material was triturated with MTBE. LCMS (ESI): m/z 398.1 [M+H]⁺.

### Step 4:1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]piperidine-4-carboxamide (B-172)

1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]piperidine-4-carboxamide (B-172, 60 mg, 0.068 mmol, 27% yield) was synthesized from N-[2-fluoro-5-[(4-oxo-1-piperidyl)sulfonylmethyl]phenyl]piperidine-4-carboxamide (B-171) and 2-[[2-(2,6-dioxo-3-piperidyl)-1-oxo-isoindolin-4-yl]amino]acetic acid (B-34) in a similar fashion to Compound A-88, except using 2 eq. B-34 and 3 eq. 1-propanephosphonic anhydride (50% in ethyl acetate). LCMS (ESI): m/z 697.2 [M+H]⁺.

### 1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]piperidine-4-carboxamide (B-175)

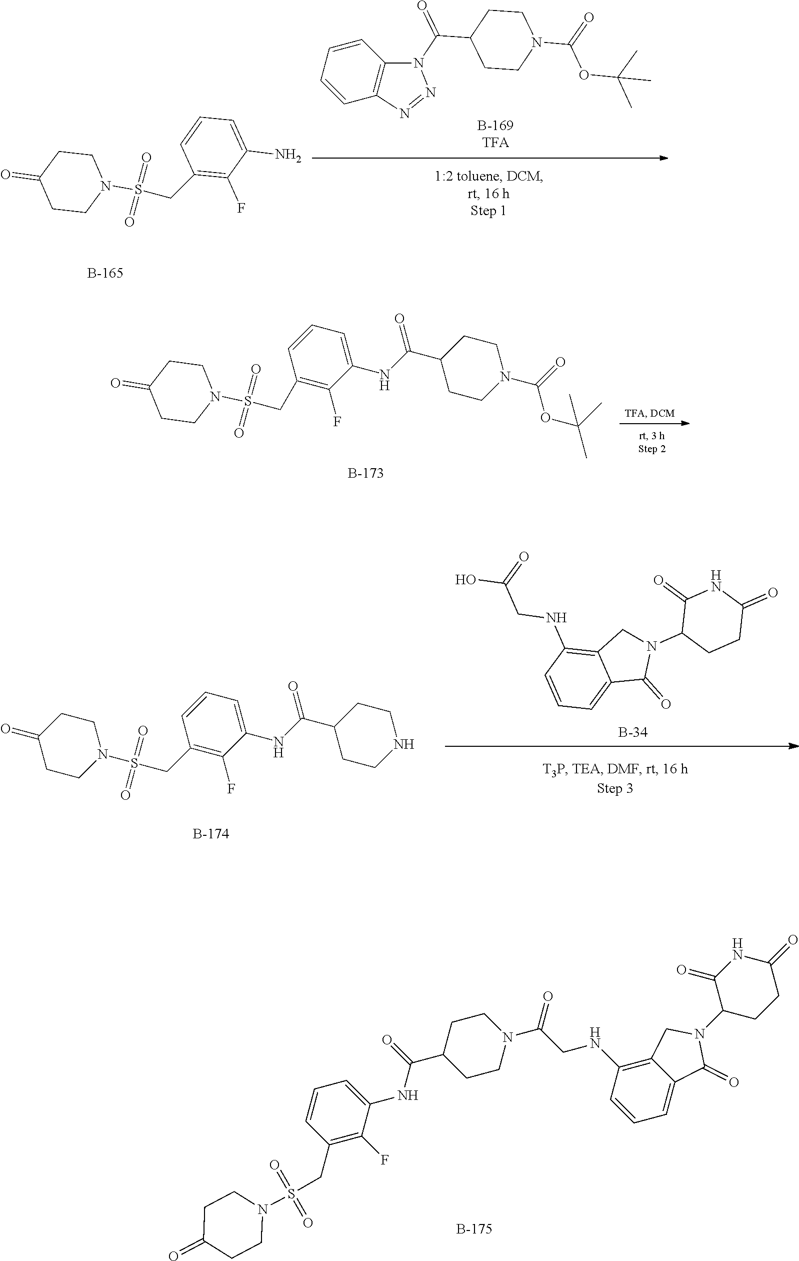

### Step 1: tert-butyl 4-[[2-fluoro-3-[(4-oxo-1-piperidyl)sulfonylmethyl]phenyl]carbamoyl]piperidine-1-carboxylate (B-173)

tert-butyl 4-[[2-fluoro-3-[(4-oxo-1-piperidyl)sulfonylmethyl]phenyl]carbamoyl]piperidine-1-carboxylate (B-173, 500 mg, 0.503 mmol, 30% yield) was synthesized from tert-butyl 4-(benzotriazole-1-carbonyl)piperidine-1-carboxylate (B-169) and 1-[(3-amino-2-fluoro-phenyl)methylsulfonyl]piperidin-4-one (B-165) in a similar fashion to Compound B-158, except using 1 eq. TFA. The material was triturated with diethyl ether. LCMS (ES−): m/z 496.2 [M−H]⁻.

### Step 2: N-[2-fluoro-3-[(4-oxo-1-piperidyl)sulfonylmethyl]phenyl]piperidine-4-carboxamide (B-174)

N-[2-fluoro-3-[(4-oxo-1-piperidyl)sulfonylmethyl]phenyl]piperidine-4-carboxamide (B-174, 400 mg, 369.37 μmol, 37% yield) was synthesized from tert-butyl 4-[[2-fluoro-3-[(4-oxo-1-piperidyl)sulfonylmethyl]phenyl]carbamoyl]piperidine-1-carboxylate (B-173) in a similar fashion to Compound A-62, except using 13 eq. TFA. The material was triturated with diethyl ether. LCMS (ES+): m/z: 398.1 [M+H]⁺.

### Step 3: 1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]piperidine-4-carboxamide (B-175)

1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]piperidine-4-carboxamide (B-175, 200 mg, 0.201 mmol, 26% yield) was synthesized from N-[2-fluoro-3-[(4-oxo-1-piperidyl)sulfonylmethyl]phenyl]piperidine-4-carboxamide (B-174) and 2-[[2-(2,6-dioxo-3-piperidyl)-1-oxo-isoindolin-4-yl]amino]acetic acid (B-34) in a similar fashion to Compound B-10, except using 1.7 eq. triethylamine and 1.2 eq. 1-propanephosphonic anhydride solution (50% in Ethyl acetate). Upon completion, the reaction was concentrated under reduced pressure and the residue purified by reverse phase column chromatography [Column: RediSep ISCO C18 (30 g); Mobile phase A: 0.1% Ammonium Acetate in MQ-water; Mobile phase B: Acetonitrile]. LCMS (ES−): m/z 695.5 [M−H]⁻.

### 3-[1-oxo-5-(4-piperidyloxy)isoindolin-2-yl]piperidine-2,6-dione (B-178)

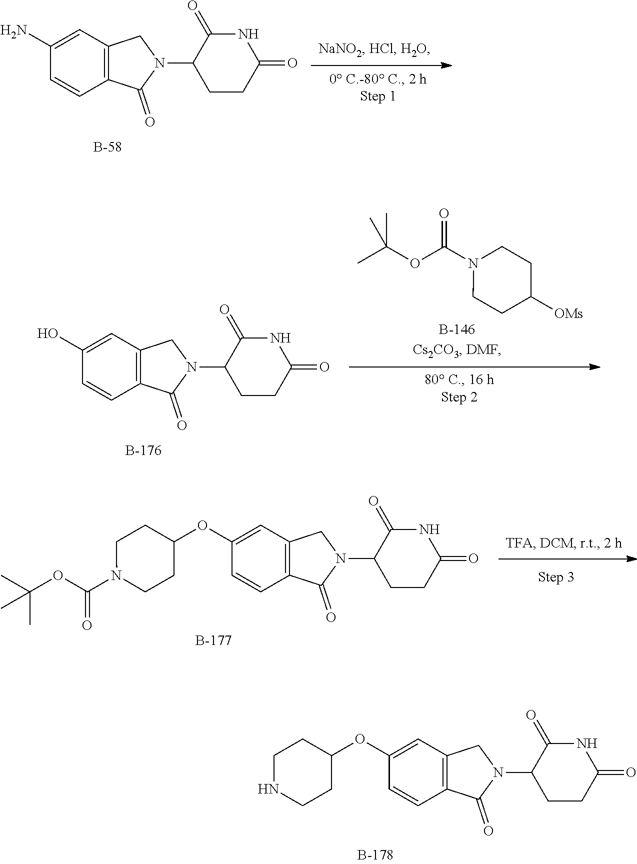

### Step 1: 3-(5-hydroxy-1-oxo-isoindolin-2-yl)piperidine-2,6-dione (B-176)

3-(5-hydroxy-1-oxo-isoindolin-2-yl)piperidine-2,6-dione (B-176, 200 mg, 716.86 μmol, 19% yield) was synthesized from 3-(5-amino-1-oxo-isoindolin-2-yl)piperidine-2,6-dione (B-58) in a similar fashion to Compound B-147, except using 23 eq. hydrochloric acid (36% w/w aq. Solution). LCMS (ES+): m/z 261.1 [M+H]⁺.

### Step 2: tert-butyl 4-[2-(2,6-dioxo-3-piperidyl)-1-oxo-isoindolin-5-yl]oxypiperidine-1-carboxylate (B-177)

tert-butyl 4-[2-(2,6-dioxo-3-piperidyl)-1-oxo-isoindolin-5-yl]oxypiperidine-1-carboxylate (B-177, 55 mg, 96.73 μmol, 25% yield) was synthesized from 3-(5-hydroxy-1-oxo-isoindolin-2-yl)piperidine-2,6-dione (B-176) and tert-butyl 4-methylsulfonyloxypiperidine-1-carboxylate (B-146) in a similar fashion to Compound B-148, LCMS (ES+): m/z 442.1 [M−H]⁻.

### Step 3: 3-[1-oxo-5-(4-piperidyloxy)isoindolin-2-yl]piperidine-2,6-dione (B-178)

3-[1-oxo-5-(4-piperidyloxy)isoindolin-2-yl]piperidine-2,6-dione (B-178, 40 mg, 74.18 μmol, 66% yield, TFA salt) was synthesized tert-butyl 4-[2-(2,6-dioxo-3-piperidyl)-1-oxo-isoindolin-5-yl]oxypiperidine-1-carboxylate (B-177) in a similar fashion to Compound A-62, except using 57 eq. TFA. LCMS (ES+): m/z 344.1 [M+H]⁺.

### 3-[5-(azetidin-3-ylamino)-3-methyl-2-oxo-benzimidazol-1-yl]piperidine-2,6-dione (B-182)

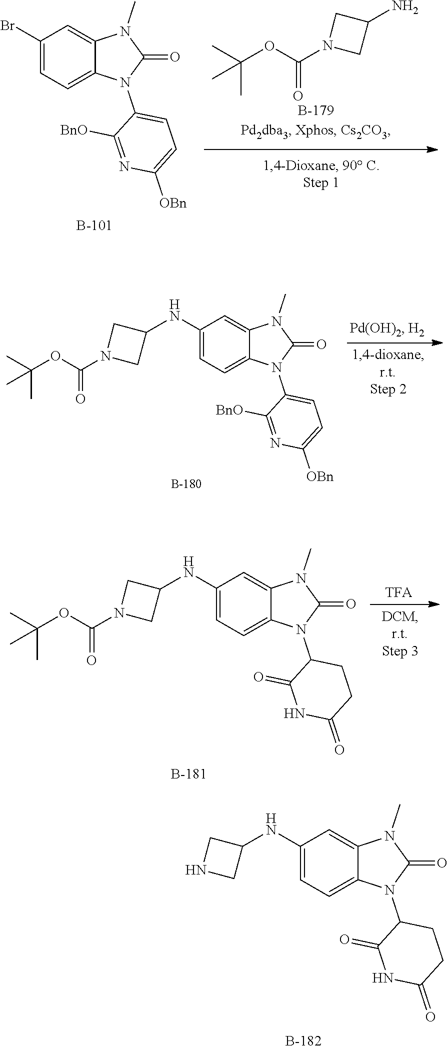

### Step 1: tert-butyl 3-[[1-(2,6-dibenzyloxy-3-pyridyl)-3-methyl-2-oxo-benzimidazol-5-yl]amino]azetidine-1-carboxylate (B-180)

Into a 20 mL sealed tube containing a well-stirred solution of 5-bromo-1-(2,6-dibenzyloxy-3-pyridyl)-3-methyl-benzimidazol-2-one (B-101, 300 mg, 0.580 mmol) in 1,4-dioxane (3 mL) were added tert-butyl 3-aminoazetidine-1-carboxylate (B-179, 250.14 mg, 1.45 mmol) and cesium carbonate (567.87 mg, 1.74 mmol). The reaction mixture was purged by bubbling nitrogen through for 5 min. Subsequently, tris(dibenzylideneacetone) dipalladium (0) (79.80 mg, 0.087 mmol) and XPhos (69.24 mg, 0.145 mmol) were added and the reaction mixture was heated to 90° C. for 16 h. The reaction mixture was cooled to room temperature and poured into water (20 mL). The aqueous layer was extracted with Ethyl acetate (2×30 mL). The organic layers were combined, dried over sodium sulfate, filtered and concentrated under reduced pressure. The residue was purified by flash silica-gel (230-400 mesh) column, eluting with 0-100% Ethyl acetate/petroleum ether to afford tert-butyl 3-[[1-(2,6-dibenzyloxy-3-pyridyl)-3-methyl-2-oxo-benzimidazol-5-yl]amino]azetidine-1-carboxylate (B-180, 280 mg, 0.437 mmol, 75% yield) as a pale yellow foam. LCMS (ESI): m/z 608.2 [M+H]⁺.

### Step 2: tert-butyl 3-[[1-(2,6-dioxo-3-piperidyl)-3-methyl-2-oxo-benzimidazol-5-yl]amino]azetidine-1-carboxylate (B-181)

tert-butyl 3-[[1-(2,6-dioxo-3-piperidyl)-3-methyl-2-oxo-benzimidazol-5-yl]amino]azetidine-1-carboxylate (B-181, 160 mg, 0.219 mmol, 48% yield) was synthesized from tert-butyl 3-[[1-(2,6-dibenzyloxy-3-pyridyl)-3-methyl-2-oxo-benzimidazol-5-yl]amino]azetidine-1-carboxylate (B-180) in a similar fashion to Compound B-103, except using 0.2 eq. palladium hydroxide on carbon (20% by weight, 50% water). The Celite pad was washed with dioxane, and the material was triturated with diethyl ether. LCMS (ESI): m/z 428.1 [M−H]⁻.

### Step 3: 3-[5-(azetidin-3-ylamino)-3-methyl-2-oxo-benzimidazol-1-yl]piperidine-2,6-dione (B-182)

3-[5-(azetidin-3-ylamino)-3-methyl-2-oxo-benzimidazol-1-yl]piperidine-2,6-dione (B-182, 45 mg, 0.071 mmol, 61% yield) was synthesized from tert-butyl 3-[[1-(2,6-dioxo-3-piperidyl)-3-methyl-2-oxo-benzimidazol-5-yl]amino]azetidine-1-carboxylate (B-181) in a similar fashion to Compound A-62, except using 28 eq. TFA. The material was triturated with diethyl ether. LCMS (ESI): m/z 330.1 [M+H]⁺.

### 3-(3-methyl-2-oxo-5-piperazin-1-yl-benzimidazol-1-yl)piperidine-2,6-dione (B-185)

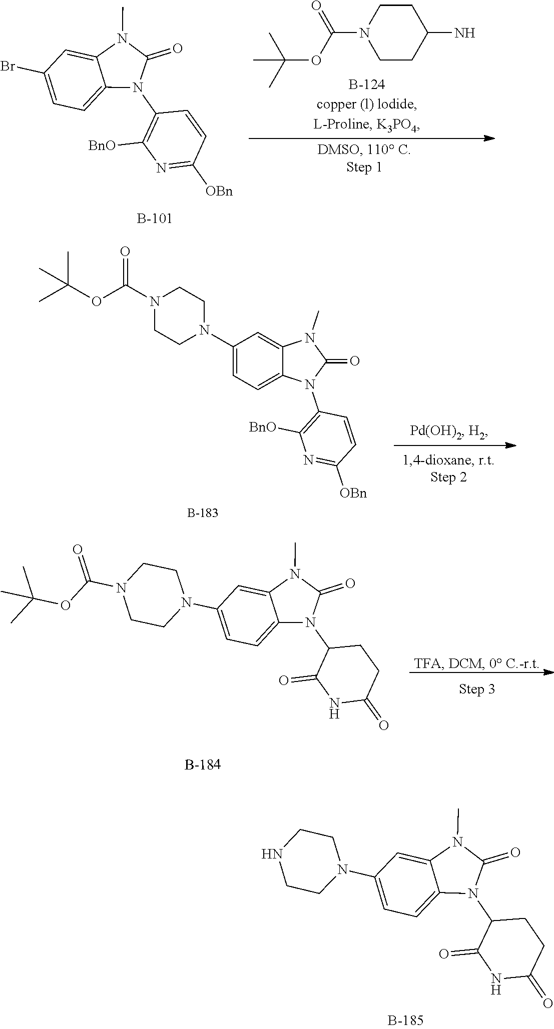 [SMILES: Cn1c(=O)n(-c2ccc(OCc3ccccc3)nc2OCc2ccccc2)c2ccc(Br)cc21 ; CC(C)(C)OC(=O)N1CCC(N)CC1 ; Cn1c(=O)n(-c2ccc(OCc3ccccc3)nc2OCc2ccccc2)c2ccc(N3CCN(C(=O)OC(C)(C)C)CC3)cc21 ; Cn1c(=O)n(C2CCC(=O)NC2=O)c2ccc(N3CCN(C(=O)OC(C)(C)C)CC3)cc21 ; Cn1c(=O)n(C2CCC(=O)NC2=O)c2ccc(N3CCNCC3)cc21]

### Step 1: tert-butyl 4-[1-(2,6-dibenzyloxy-3-pyridyl)-3-methyl-2-oxo-benzimidazol-5-yl]piperazine-1-carboxylate (B-183)

Into a 50 mL sealed tube containing a well-stirred solution of 5-bromo-1-(2,6-dibenzyloxy-3-pyridyl)-3-methyl-benzimidazol-2-one (B-101, 300 mg, 0.580 mmol) and tert-butyl piperazine-1-carboxylate (B-124, 324.61 mg, 1.74 mmol) in anhydrous DMSO (3 mL) were added copper (I) iodide (110.64 mg, 0.580 mmol, 19.69 μL) and potassium phosphate tribasic (anhydrous) (123.32 mg, 0.580 mmol). The mixture was purged by bubbling nitrogen gas through the reaction mixture for 10 min. Subsequently, L-proline (66.89 mg, 0.580 mmol) was added and the resulting mixture was heated at 110° C. for 16 h. The reaction mixture was poured into water (25 mL) and extracted with Ethyl acetate (2×30 mL). The combined organic layer was washed with brine (10 mL), fried over sodium sulfate, filtered and concentrated under reduced pressure. The residue was purified by flash silica-gel (230-400 mesh) column, eluting with 0-100% Ethyl acetate/petroleum ether to afford tert-butyl 4-[1-(2,6-dibenzyloxy-3-pyridyl)-3-methyl-2-oxo-benzimidazol-5-yl]piperazine-1-carboxylate (B-183, 100 mg, 0.127 mmol, 22% yield) as a pale-yellow gum. LCMS (ESI): m/z 622.20 [M+H]⁺.

### Step 2: tert-butyl 4-[1-(2,6-dioxo-3-piperidyl)-3-methyl-2-oxo-benzimidazol-5-yl]piperazine-1-carboxylate (B-184)

ter-butyl 4-[1-(2,6-dioxo-3-piperidyl)-3-methyl-2-oxo-benzimidazol-5-yl]piperazine-1-carboxylate (B-184, 80 mg, 0.172 mmol, 80% yield) was synthesized from tert-butyl 4-[1-(2,6-dibenzyloxy-3-pyridyl)-3-methyl-2-oxo-benzimidazol-5-yl]piperazine-1-carboxylate (B-183) in a similar fashion to Compound B-103, except using 0.9 eq. palladium hydroxide on carbon (20% by weight, 50% water). The Celite pad was washed with dioxane, and the material was triturated with diethyl ether. LCMS (ESI): m/z 444.20 [M+H]⁺.

### Step 3: 3-(3-methyl-2-oxo-5-piperazin-1-yl-benzimidazol-1-yl)piperidine-2,6-dione (B-185)

3-(3-methyl-2-oxo-5-piperazin-1-yl-benzimidazol-1-yl)piperidine-2,6-dione (B-185, 80 mg, 0,157 mmol, 87% yield, TFA salt) was synthesized from tert-butyl 4-[1-(2,6-dioxo-3-piperidyl)-3-methyl-2-oxo-benzimidazol-5-yl]piperazine-1-carboxy late (B-185) in a similar fashion to Compound A-62, except using 36 eq. TFA. The material was triturated with diethyl ether. LCMS (ESI): m/z 344.10 [M+H]⁺.

### 3-[3-methyl-2-oxo-5-(4-piperidylamino)benzimidazol-1-yl]piperidine-2,6-dione (B-188)

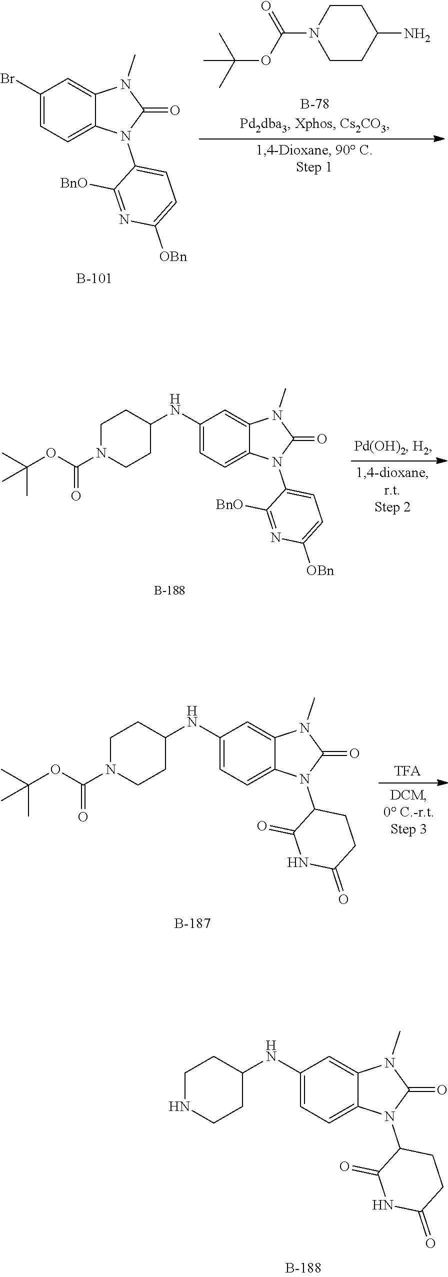 [SMILES: Cn1c(=O)n(-c2ccc(OCc3ccccc3)nc2OCc2ccccc2)c2ccc(Br)cc21 ; CC(C)(C)OC(=O)N1CCC(N)CC1 ; Cn1c(=O)n(-c2ccc(OCc3ccccc3)nc2OCc2ccccc2)c2ccc(NC3CCN(C(=O)OC(C)(C)C)CC3)cc21 ; Cn1c(=O)n(C2CCC(=O)NC2=O)c2ccc(NC3CCN(C(=O)OC(C)(C)C)CC3)cc21 ; Cn1c(=O)n(C2CCC(=O)NC2=O)c2ccc(NC3CCNCC3)cc21]

### Step 1: tert-butyl 4-[[1-(2,6-dibenzyloxy-3-pyridyl)-3-methyl-2-oxo-benzimidazol-5-yl]amino]piperidine-1-carboxylate (B-186)

tert-butyl 4-[[1-(2,6-dibenzyloxy-3-pyridyl)-3-methyl-2-oxo-benzimidazol-5-yl]amino]piperidine-1-carboxylate (B-186, 400 mg, 0.536 mmol, 55% yield) was synthesized from 5-bromo-1-(2,6-dibenzyloxy-3-pyridyl)-3-methyl-benzimidazol-2-one (B-101) and tert-butyl 4-aminopiperidine-1-carboxylate (B-78) in a similar fashion to Compound B-180, except using 1.3 eq. B-78 and 0.3 eq. XPhos. LCMS (ESI): m/z 636.3 [M+H]⁺.

### Step 2: tert-butyl 4-[[1-(2,6-dioxo-3-piperidyl)-3-methyl-2-oxo-benzimidazol-5-yl]amino]piperidine-1-carboxylate (B-187)

tert-butyl 4-[[1-(2,6-dioxo-3-piperidyl)-3-methyl-2-oxo-benzimidazol-5-yl]amino]piperidine-1-carboxylate (B-187, 250 mg, 0.408 mmol, 65% yield) was synthesized from tert-butyl 4-[[1-(2,6-dibenzyloxy-3-pyridyl)-3-methyl-2-oxo-benzimidazol-5-yl]amino]piperidine-1-carboxylate (B-186) in a similar fashion to Compound B-103, except using 0.3 eq. palladium hydroxide on carbon (20% by weight, 50% water). The Celite pad was washed with dioxane, and the material was triturated with diethyl ether. LCMS (ESI): m/z 458.0 [M+H]⁺.

### Step 3: 3-[3-methyl-2-oxo-5-(4-piperidylamino)benzimidazol-1-yl]piperidine-2,6-dione (B-188)

3-[3-methyl-2-oxo-5-(4-piperidylamino)benzimidazol-1-yl]piperidine-2,6-dione (B-188, 220 mg, 0.258 mmol, 47% yield) was synthesized from tert-butyl 4-[[1-(2,6-dioxo-3-piperidyl)-3-methyl-2-oxo-benzimidazol-5-yl]amino]piperidine-1-carboxylate (B-187) in a similar fashion to Compound A-62, except using 24 eq. TFA. The material was triturated with diethyl ether. LCMS (ESI): m/z 358.0 [M+H]⁺.

### 3-[2-oxo-6-(4-piperidyl)benzo[cd]indol-1-yl]piperidine-2,6-dione (B-194)

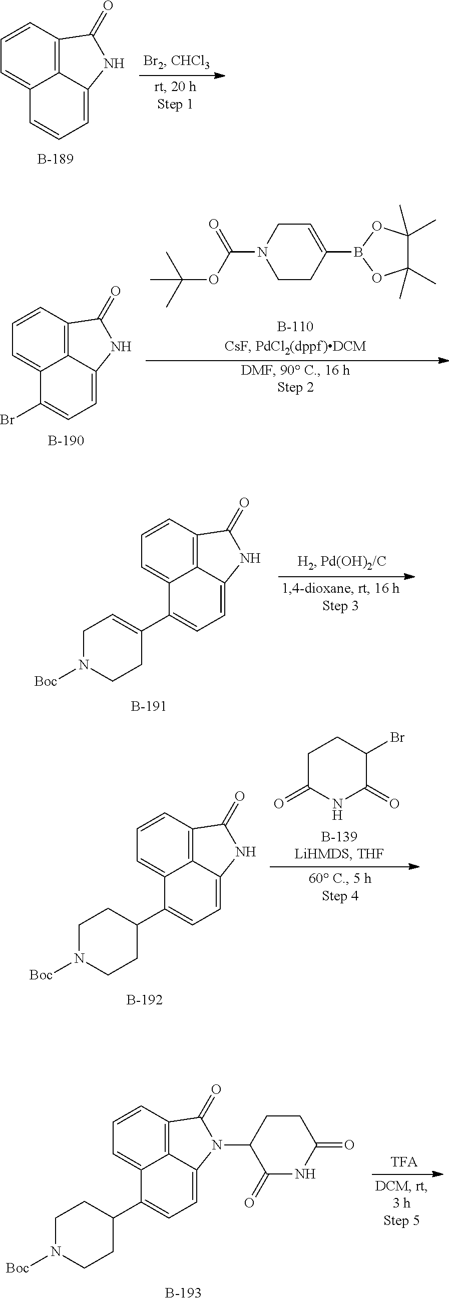

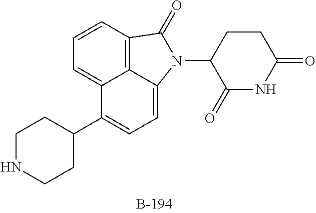 [SMILES: O=C1CCC(N2C(=O)c3cccc4c(C5CCNCC5)ccc2c34)C(=O)N1]

### Step 1: 6-bromo-1H-benzo[c]indol-2-one (B-190)

Into a 1 L two neck round bottom flask containing a well-stirred solution of 1H-benzo[cd]indol-2-one (B-189, 5 g, 29.55 mmol) in CHCl₃ (300 mL) was added bromine (3.59 g, 44.33 mmol, 2.41 mL) at 0° C. The reaction mixture was stirred at room temperature for 20 h. The reaction mixture was quenched with sodium thiosulfate solution (200 mL) at 0° C., and the yellow solid was filtered through sintered funnel, washed with cold water (250 mL) and diethyl ether (150 mL) to afford 6-bromo-1H-benzo[cd]indol-2-one (B-190, 5.8 g, 21.51 mmol, 73% yield) as a yellow solid. The material was used in the next step without further purification. LCMS (ES+): m/z 250.1 [M+H]⁺.

### Step 2: tert-butyl 4-(2-oxo-1H-benzo[cd]indol-6-yl)-3,6-dihydro-2H-pyridine-1-carboxylate (B-191)

Into a 250 mL pressure tube containing a well-stirred suspension of 6-bromo-1H-benzo[cd]indol-2-one (B-190, 4 g, 16.12 mmol, 60.24 μL) in DMF (30 mL) was added tert-butyl 4-(4,4,5,5-tetramethyl-1,3,2-dioxaborolan-2-yl)-3,6-dihydro-2H-pyridine-1-carboxylate (B-110, 5.48 g, 17.74 mmol) and cesium fluoride (4.90 g, 32.25 mmol, 1.19 mL). The mixture was purged by bubbling nitrogen gas through for 5 min. Then [1,1′-bis(diphenylphosphino)ferrocene]dichloropalladium(II), complex with dichloromethane (1.5 g, 2.42 mmol) was added while degassing and the pressure tube was sealed. The reaction mixture was heated at 90° C. for 16 h. The reaction mixture was concentrated under reduced pressure and the residue was purified by flash silica gel column chromatography (50-60% Ethyl acetate in petroleum ether) to afford tert-butyl 4-(2-oxo-1H-benzo[cd]indol-6-yl)-3,6-dihydro-2H-pyridine-1-carboxylate (B-191, 2.1 g, 3.23 mmol, 31% yield) as a pale yellow solid. LCMS (ES+): m/z 351.2 [M+H]⁺.

### Step 3: tert-butyl 4-(2-oxo-1H-benzo[cd]indol-6-yl)piperidine-1-carboxylate (B-192)

tert-butyl 4-(2-oxo-1H-benzo[cd]indol-6-yl)piperidine-1-carboxylate (B-192, 2 g, 4.90 mmol, 78% yield) was synthesized from tert-butyl 4-(2-oxo-1H-benzo[cd]indol-6-yl)-3,6-dihydro-2H-pyridine-1-carboxylate (B-191) in a similar fashion to Compound B-103, except using 0.2 eq. palladium hydroxide on carbon (20% by weight, 50% water). LCMS (ES+): m/z 353.1 [M+H]⁺.

### Step 4: tert-butyl 4-[1-(2,6-dioxo-3-piperidyl)-2-oxo-benzo[cd]indol-6-yl]piperidine-1-carboxylate (B-193)

Into a 250 mL two neck round bottom flask containing a well-stirred solution of tert-butyl 4-(2-oxo-1H-benzo[cd]indol-6-yl)piperidine-1-carboxylate (B-192, 2 g, 5.67 mmol) in THF (20 mL) was added a solution of lithium bis(trimethylsilyl)amide (1.0 M in THF) (5.67 mmol, 12 mL) at 0° C. After 30 min, 3-bromopiperidine-2,6-dione (B-139, 1.09 g, 5.67 mmol) was added at 0° C. in portions. Then the reaction mixture was stirred at 60° C. for 5 h. Then the reaction mixture was cooled to 0° C. and 1.5 N HCl (4 mL) added to adjust the pH to 3-4. The reaction mixture was diluted with Ethyl acetate (400 mL). The organic layer was washed with water (200 mL) and brine solution (150 mL). The solvent was dried over anhydrous Sodium sulfate, filtered and concentrated. The residue was purified by reverse phase prep HPLC (Column: YMC C-18 (150×30 mm), 5 μm, Mobile phase A: 0.1% TFA in water; Mobile phase B: MeCN) to obtain tert-butyl 4-[1-(2,6-dioxo-3-piperidyl)-2-oxo-benzo[cd]indol-6-yl]piperidine-1-carboxylate (B-193, 1 g, 1.69 mmol, 30% yield) as a pale yellow solid. LCMS (ES+): m/z 462.2 [M+H]⁺.

### Step 5: 3-[2-oxo-6-(4-piperidyl)benzo[cd]indol-1-yl]piperidine-2,6-dione (B-194)

3-[2-oxo-6-(4-piperidyl)benzo[cd]indol-1-yl]piperidine-2,6-dione (B-194, 80 mg, 163.21 μmol, 76% yield, TFA salt) was synthesized from tert-butyl 4-[1-(2,6-dioxo-3-piperidyl)-2-oxo-benzo[cd]indol-6-yl]piperidine-1-carboxylate (B-193) in a similar fashion to Compound A-62, except using 30 eq. TFA. The material was triturated with diethyl ether. LCMS (ES+): m/z 364.1 [M+H]⁺.

### 2-[4-[1-(2,6-dioxo-3-piperidyl)-2-oxo-benzo[cd]indol-6-yl]-1-piperidyl]acetic acid (B-196)

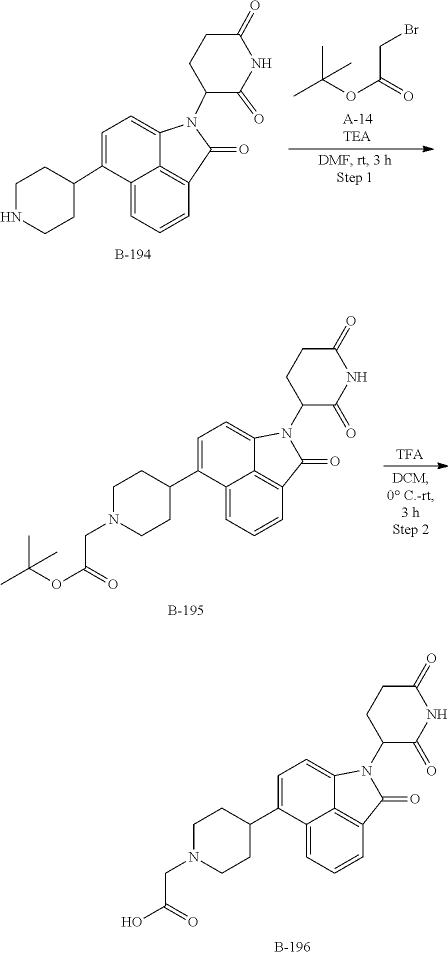

### Step 1: tert-butyl 2-[4-[1-(2,6-dioxo-3-piperidyl)-2-oxo-benzo[cd]indol-6-yl]-1-piperidyl]acetate (B-195)

Into a 10 mL single neck round bottom flask containing a well-stirred solution of 3-[2-oxo-6-(4-piperidyl)benzo[cd]indol-1-yl]piperidine-2,6-dione (B-194, 65 mg, 136.15 μmol, TFA salt) in DMF (1 mL) was added triethylamine (68.88 mg, 680.73 μmol, 94.88 μL) at room temperature. Then, tert-butyl 2-bromoacetate (A-14, 31.87 mg, 163.37 μmol, 23.96 μL) was added at 0° C. The reaction mixture was stirred for 3 h at room temperature. The reaction was quenched with cold water (0.5 mL) and the precipitate was filtered and washed with cold water (0.5 mL) and diethyl ether (1 mL) to afford tert-butyl 2-[4-[1-(2,6-dioxo-3-piperidyl)-2-oxo-benzo[cd]indol-6-yl]-1-piperidyl]acetate (B-195, 64 mg, 132.14 μmol, 97% yield) as a pale yellow solid. The material was used in the next step without further purification. LCMS (ES+): m/z 478.1 [M+H]⁺.

### Step 2: 2-[4-[1-(2,6-dioxo-3-piperidyl)-2-oxo-benzo[cd]indol-6-yl]-1-piperidyl]acetic acid (B-196)

2-[4-[1-(2,6-dioxo-3-piperidyl)-2-oxo-benzo[cd]indol-6-yl]-1-piperidyl]acetic acid (B-196, 71 mg, 128.75 μmol, 88% yield, TFA salt) was synthesized from tert-butyl 2-[4-[1-(2,6-dioxo-3-piperidyl)-2-oxo-benzo[cd]indol-6-yl]-1-piperidyl]acetate (B-195) in a similar fashion to Compound A-26, except using 23 eq. TFA. LCMS (ES+): m/z 422.1 [M+H]⁺.

### N-[[2-(2,6-dioxo-3-piperidyl)-1-oxo-isoindolin-4-yl]methyl]piperidine-4-carboxamide (B-199)

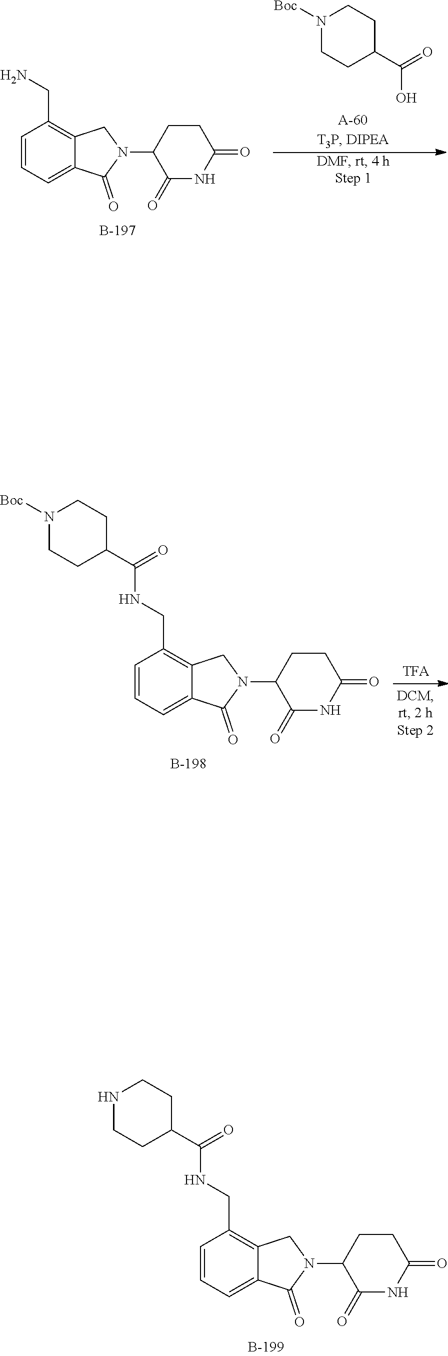 [SMILES: NCc1cccc2c1CN(C1CCC(=O)NC1=O)C2=O ; CC(C)(C)OC(=O)N1CCC(C(=O)O)CC1 ; CC(C)(C)OC(=O)N1CCC(C(=O)NCc2cccc3c2CN(C2CCC(=O)NC2=O)C3=O)CC1 ; O=C1CCC(N2Cc3c(CNC(=O)C4CCNCC4)cccc3C2=O)C(=O)N1]

### Step 1: tert-butyl 4-[[2-(2,6-dioxo-3-piperidyl)-1-oxo-isoindolin-4-yl]methylcarbamoyl]piperidine-1-carboxylate (B-198)

tert-butyl 4-[[2-(2,6-dioxo-3-piperidyl)-1-oxo-isoindolin-4-yl]methylcarbamoyl]piperidine-1-carboxylate (B-198, 20 mg, 39.21 μmol, 36% yield) was synthesized from 1-tert-butoxycarbonylpiperidine-4-carboxylic acid (A-60) and 3-[4-(aminomethyl)-1-oxo-isoindolin-2-yl]piperidine-2,6-dione (B-197) in a similar fashion to Compound A-88, except using 1 eq. B-197, 3 eq. DIPEA and 1 eq. 1-propanephosphonic anhydride (50% in ethyl acetate). LCMS (ES+): m/z 385.1 [M−Boc+H]⁺.

### Step 2: N-[[2-(2,6-dioxo-3-piperidyl)-1-oxo-isoindolin-4-yl]methyl]piperidine-4-carboxamide (B-199)

N-[[2-(2,6-dioxo-3-piperidyl)-1-oxo-isoindolin-4-yl]methyl]piperidine-4-carboxamide (B-199, 15 mg, 28.83 μmol, 70% yield, TFA salt) was synthesized from tert-butyl 4-[[2-(2,6-dioxo-3-piperidyl)-1-oxo-isoindolin-4-yl]methylcarbamoyl]piperidine-1-carboxylate (B-198) in a similar fashion to Compound A-62, except using 5 eq. TFA. The material was triturated with diethyl ether. LCMS (ES+): m/z 385.1 [M+H]⁺.

### 3-[4-[[2-(4-hydroxy-1-piperidyl)-2-oxo-ethyl]amino]-1-oxo-isoindolin-2-yl]piperidine-2,6-dione (B-201)

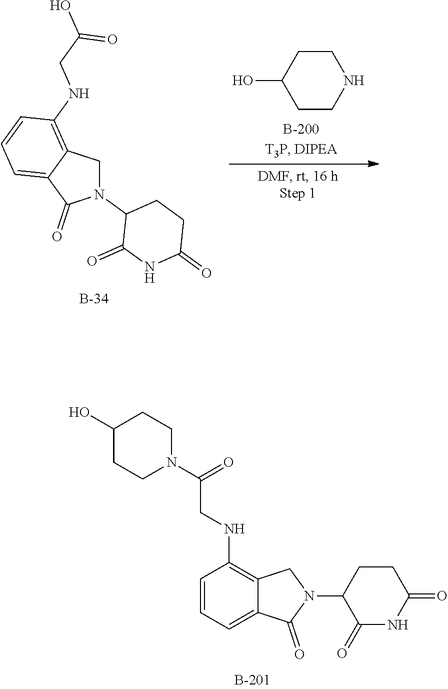

### Step 1: 3-[4-[[2-(4-hydroxy-1-piperidyl)-2-oxo-ethyl]amino]-1-oxo-isoindolin-2-yl]piperidine-2,6-dione (B-201)

3-[4-[[2-(4-hydroxy-1-piperidyl)-2-oxo-ethyl]amino]-1-oxo-isoindolin-2-yl]piperidine-2,6-dione (B-201, 220 mg, 461.51 μmol, 59% yield) was synthesized from 2-[[2-(2,6-dioxo-3-piperidyl)-1-oxo-isoindolin-4-yl]amino]acetic acid (B-34) and piperidin-4-ol (B-200) in a similar fashion to Compound A-88, except using 1.5 eq. B-200, 4 eq. DIPEA and 1.5 eq. 1-propanephosphonic anhydride (50% in ethyl acetate). The material was purified by flash silica-gel (230-400 mesh) column chromatography (0-15% MeOH in DCM). LCMS (ES+): m/z 401.1 [M+H]⁺.

### (1r,4r)-4-((2-(2,6-dioxopiperidin-3-yl)-1-oxoisoindolin-4-yl)carbamoyl)cyclohexane-1-carboxylic acid (B-204)

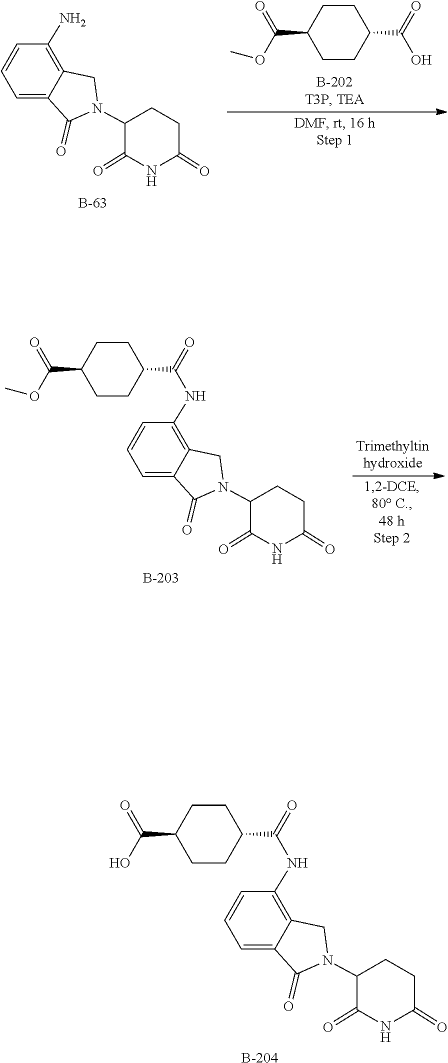

### Step 1: methyl (1r,4r)-4-((2-(2,6-dioxopiperidin-3-yl)-1-oxoisoindolin-4-yl)carbamoyl)cyclohexane-1-carboxylate (B-203)

methyl (1r,4r)-4-((2-(2,6-dioxopiperidin-3-yl)-1-oxoisoindolin-4-yl)carbamoyl)cyclohexane-1-carboxylate (B-203, 600 mg, 566.24 μmol, 37% yield) was synthesized from 3-(4-amino-1-oxo-isoindolin-2-yl)piperidine-2,6-dione (B-63) and (1r,4r)-4-(methoxycarbonyl)cyclohexane-1-carboxylic acid (B-202) in a similar fashion to Compound B-10, except using 3 eq. 1-propanephosphonic anhydride solution (50% in Ethyl acetate). LCMS (ESI): m/z 428.1 [M+H]⁺.

### Step 2: (1r,4r)-4-((2-(2,6-dioxopiperidin-3-yl)-1-oxoisoindolin-4-yl)carbamoyl)cyclohexane-1-carboxylic acid (B-204)

(1r,4r)-4-((2-(2,6-dioxopiperidin-3-yl)-1-oxoisoindolin-4-yl)carbamoyl)cyclohexane-1-carboxylic acid (B-204, 160 mg, 380.12 μmol, 67% yield) was synthesized from methyl (1r,4r)-4-((2-(2,6-dioxopiperidin-3-yl)-1-oxoisoindolin-4-yl)carbamoyl)cyclohexane-1-carboxylate (B-203) in a similar fashion to Compound B-11, except using 5 eq. trimethyltin hydroxide. LCMS (ESI): m/z 414.0 [M+H]⁺.

### 3-[3-isopropyl-2-oxo-5-(4-piperidyl)benzimidazol-1-yl]piperidine-2,6-dione (B-212)

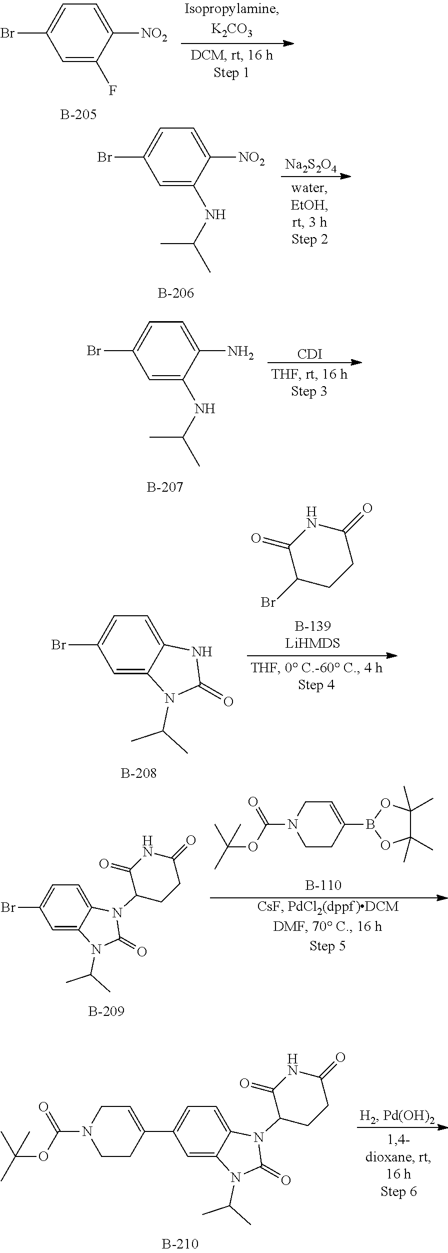

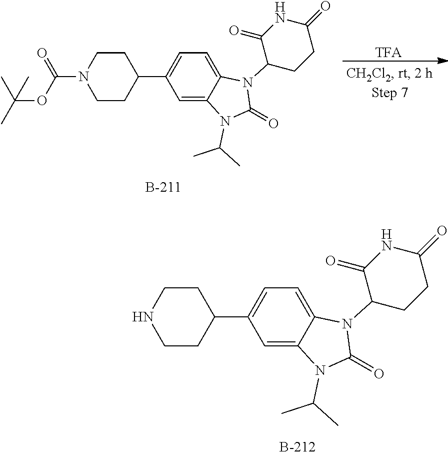 [SMILES: "TFA" ; CC(C)n1c(=O)n(C2CCC(=O)NC2=O)c2ccc(C3CCN(C(=O)OC(C)(C)C)CC3)cc21 ; CC(C)n1c(=O)n(C2CCC(=O)NC2=O)c2ccc(C3CCNCC3)cc21]

### Step 1: 5-bromo-N-isopropyl-2-nitroaniline (B-206)

Into a 100 mL three neck round bottom flask containing a well-stirred solution of 4-bromo-2-fluoro-1-nitrobenzene (B-205, 2.4 g, 10.91 mmol) in DCM (25 mL) was added potassium carbonate (3.02 g, 21.82 mmol) followed by dropwise addition of isopropylamine (644.85 mg, 10.91 mmol, 933.21 μL) over a period of 5 min. The reaction mixture was stirred at room temperature for 16 h. The reaction mixture was diluted with DCM (50 mL) and washed with water (3×40 mL). The organic layer was dried over anhydrous sodium sulfate and concentrated to obtain 5-bromo-N-isopropyl-2-nitroaniline (B-206, 2.8 g, 10.78 mmol, 99% yield) as a bright yellow solid. The material was used in the next step without further purification. LCMS (ES+): m/z 258.9 [M+H]⁺.

### Step 2: 4-bromo-N₂-isopropylbenzene-1,2-diamine (B-207)

Into a 250 mL three necked round bottom flask, containing a well-stirred solution of 5-bromo-N-isopropyl-2-nitroaniline (B-206, 2.8 g, 10.81 mmol) in ethanol (60 mL) was added a solution of sodium dithionite (8.47 g, 48.63 mmol) in water (25 mL). The reaction mixture was stirred for 3 h. The solvent was removed under reduced pressure and the residue was dissolved in Ethyl acetate (50 mL). The organic layer was washed with water (3×40 mL) and dried over anhydrous sodium sulfate. The solvent was removed under reduced pressure to obtain 4-bromo-N2-isopropylbenzene-1,2-diamine (B-207, 2.2 g, 8.41 mmol, 78% yield) as a pale-yellow liquid. The material was used in the next step without further purification. LCMS (ES+): m/z 231.1 [M+H]⁺.

### Step 3: 5-Bromo-3-isopropyl-1H-benzimidazol-2-one (B-208)

Into a 100 mL three neck round bottom flask containing a well-stirred solution of 4-bromo-N2-isopropylbenzene-1,2-diamine (B-207, 2.2 g, 9.60 mmol) in THF (25 mL) was added CDI (2.34 g, 14.40 mmol). After 16 h, the reaction mixture was diluted with Ethyl acetate (50 mL) and was washed with water (3×30 mL). The organic layer was dried over anhydrous sodium sulfate and concentrated. The residue was purified by flash silica gel column chromatography (40% Ethyl acetate in petroleum ether) to afford 5-bromo-3-isopropyl-1H-benzimidazol-2-one (B-208, 1.48 g, 5.80 mmol, 60% yield) as a white solid. LCMS (ES+): m/z 257.0 [M+H]⁺.

### Step 4: 3-(5-bromo-3-isopropyl-2-oxo-benzimidazol-1-yl)piperidine-2,6-dione (B-209)

3-(5-bromo-3-isopropyl-2-oxo-benzimidazol-1-yl)piperidine-2,6-dione (B-209, 480 mg, 1.31 mmol, 23% yield) was synthesized from 5-bromo-3-isopropyl-1H-benzimidazol-2-one (B-208) and 3-bromopiperidine-2,6-dione (B-139) in a similar fashion to Compound B-193, except the material was purified by flash silica gel column chromatography (50% Ethyl acetate in petroleum ether). LCMS (ES+): m/z 366.0 [M+H]⁺.

### Step 5: tert-butyl 4-[1-(2,6-dioxo-3-piperidyl)-3-isopropyl-2-oxo-benzimidazol-5-yl]-3,6-dihydro-2H-pyridine-1-carboxylate (B-210)

tert-butyl 4-[1-(2,6-dioxo-3-piperidyl)-3-isopropyl-2-oxo-benzimidazol-5-yl]-3,6-dihydro-2H-pyridine-1-carboxylate (B-210, 520 mg, 671.29 μmol, 49% yield) was synthesized from 3-(5-bromo-3-isopropyl-2-oxo-benzimidazol-1-yl)piperidine-2,6-dione (B-209) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 1 eq. B-110, CsF and 1 eq. 1 eq. [1,1′-Bis(diphenylphosphino)ferrocene]dichloropalladium(II) complex with dichloromethane. The reaction was heated at 70° C. Upon completion, the mixture was filtered through Celite and washed with Ethyl acetate (35 mL). The organic layer was washed with water (3×25 mL), dried over anhydrous sodium sulfate and concentrated under reduced pressure. The material was used without further purification. LCMS (ES+): m/z 469.1 [M+H]⁺.

### Step 6: tert-butyl 4-[1-(2,6-dioxo-3-piperidyl)-3-isopropyl-2-oxo-benzimidazol-5-yl]piperidine-1-carboxylate (B-211)

tert-butyl 4-[1-(2,6-dioxo-3-piperidyl)-3-isopropyl-2-oxo-benzimidazol-5-yl]piperidine-1-carboxylate (B-211, 345 mg, 554.29 μmol, 74% yield) was synthesized from tert-butyl 4-[1-(2,6-dioxo-3-piperidyl)-3-isopropyl-2-oxo-benzimidazol-5-yl]-3,6-dihydro-2H-pyridine-1-carboxylate (B-210) in a similar fashion to Compound B-103, except the material was purified by flash silica gel column chromatography (70% Ethyl acetate in petroleum ether). LCMS (ES+): m/z 415.1 [M−tBu+H]⁺.

### Step 7: 3-[3-isopropyl-2-oxo-5-(4-piperidyl)benzimidazol-1-yl]piperidine-2,6-dione (B-212)

3-[3-isopropyl-2-oxo-5-(4-piperidyl)benzimidazol-1-yl]piperidine-2,6-dione (B-212, 335 mg, 484.08 μmol, 67% yield, TFA salt) was synthesized from tert-butyl 4-[1-(2,6-dioxo-3-piperidyl)-3-isopropyl-2-oxo-benzimidazol-5-yl]piperidine-1-carboxylate (B-211) in a similar fashion to Compound A-62, except using 6 eq. TFA. The material was triturated with diethyl ether. LCMS (ES+): m/z 371.2 [M+H]⁺.

### 3-[3-ethyl-2-oxo-5-(4-piperidyl)benzimidazol-1-yl]piperidine-2,6-dione (B-219)

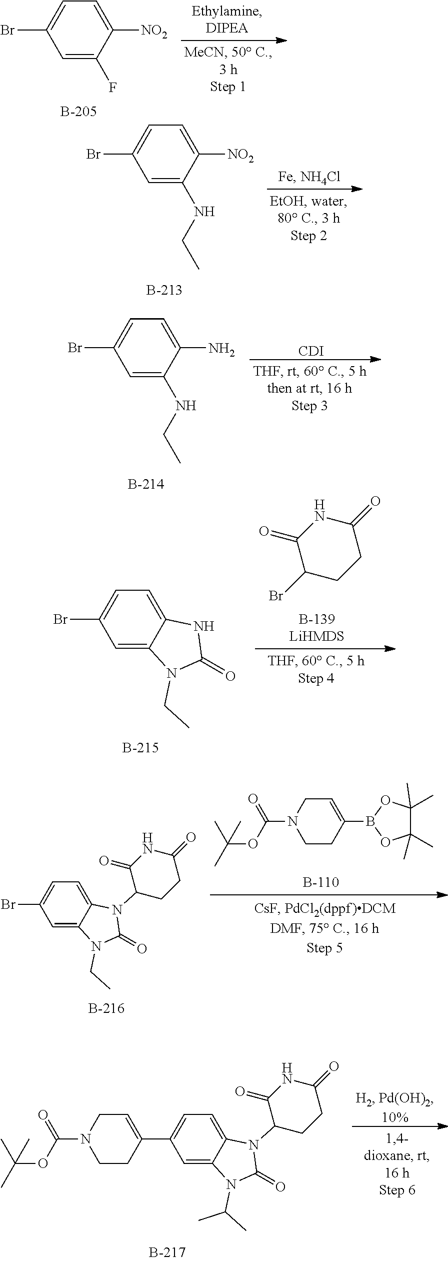

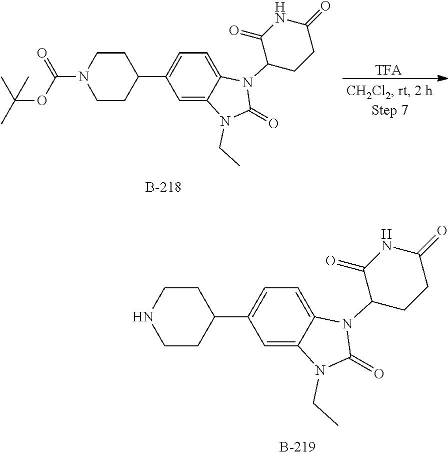 [SMILES: "TFA" ; CCn1c(=O)n(C2CCC(=O)NC2=O)c2ccc(C3CCN(C(=O)OC(C)(C)C)CC3)cc21 ; CCn1c(=O)n(C2CCC(=O)NC2=O)c2ccc(C3CCNCC3)cc21]

### Step 1: 5-bromo-N-ethyl-2-nitroaniline (B-213)

In a sealed tube, a solution of 4-bromo-2-fluoro-1-nitrobenzene (B-205, 2 g, 9.09 mmol) in Acetonitrile (10 mL), was treated with N,N-Diisopropylethylamine (1.17 g, 9.09 mmol, 1.58 mL) and ethylamine hydrochloride (741.33 mg, 9.09 mmol, 607.65 μL) and the reaction mixture was stirred at 50° C. for 3 h. The reaction mixture was quenched with ice water and the precipitate was filtered and washed with diethyl ether and dried under vacuum to afford 5-bromo-N-ethyl-2-nitroaniline (B-213, 1.75 g, 7.07 mmol, 78% yield) as a yellow solid. LCMS (ES+): m/z 245.0 [M+H]⁺.

### Step 2: 5-bromo-N₁-ethylbenzene-1,2-diamine (B-214)

5-bromo-N₁-ethylbenzene-1,2-diamine (B-214, 1.35 g, 6.15 mmol, 86% yield) was synthesized from 5-bromo-N-ethyl-2-nitroaniline (B-213) in a similar fashion to Compound B-99, except the Celite was washed with Ethyl Acetate. The filtrate was concentrated under reduced pressure and the aqueous layer was extracted with ethyl acetate. The combined organic layers were washed with water and brine, dried over sodium sulfate and filtered. The solvent was removed under reduced pressure. LCMS (ES+): m/z 217.1 [M+H]⁺.

### Step 3: 5-bromo-3-ethyl-1H-benzimidazol-2-one (B-215)

5-bromo-3-ethyl-1H-benzimidazol-2-one (B-215, 1.1 g, 4.52 mmol, 72% yield) was synthesized from 5-bromo-N₁-ethylbenzene-1,2-diamine (B-214) in a similar fashion to Compound B-208, except using 1.1 eq. CDI and heating at 70° C. for 5 h. LCMS (ES+): m/z 242.9 [M+H]⁺.

### Step 4: 3-(5-bromo-3-ethyl-2-oxo-benzimidazol-1-yl)piperidine-2,6-dione (B-216)

3-(5-bromo-3-ethyl-2-oxo-benzimidazol-1-yl)piperidine-2,6-dione (B-216, 520 mg, 1.45 mmol, 32% yield) was synthesized from 5-bromo-3-ethyl-1H-benzimidazol-2-one (B-215) and 3-bromopiperidine-2,6-dione (B-139) in a similar fashion to Compound B-193, except using 1.2 eq. lithium bis(trimethylsilyl)amide (1.0 M in THF) and 1.5 eq. B-139. The material was purified by flash silica gel column chromatography (40-60% Ethyl acetate in petroleum ether), LCMS (ES+): m/z 355.0 [M+H]⁺.

### Step 5: tert-butyl 4-[1-(2,6-dioxo-3-piperidyl)-3-ethyl-2-oxo-benzimidazol-5-yl]-3,6-dihydro-2H-pyridine-1-carboxylate (B-217)

tert-butyl 4-[1-(2,6-dioxo-3-piperidyl)-3-ethyl-2-oxo-benzimidazol-5-yl]-3,6-dihydro-2H-pyridine-1-carboxylate (B-217, 330 mg, 384.80 μmol, 26% yield) was synthesized from 3-(5-bromo-3-ethyl-2-oxo-benzimidazol-1-yl)piperidine-2,6-dione (B-216) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 1.5 eq. B-110, 2 eq. CsF and 0.15 eq. [1,1′-Bis(diphenylphosphino)ferrocene]dichloropalladium(II) complex with dichloromethane. The reaction was heated at heated at 75° C. Upon completion, the mixture was filtered through Celite, washing with ethyl acetate. The filtrate was washed with water and brine and dried over sodium sulfate. The solvent was removed under reduced pressure and the residue was purified by flash silica gel column chromatography (30-70% Ethyl acetate in petroleum ether). LCMS (ES+): m/z 455.1 [M+H]⁺.

### Step 6: tert-butyl 4-[1-(2,6-dioxo-3-piperidyl)-3-ethyl-2-oxo-benzimidazol-5-yl]piperidine-1-carboxylate (B-218)

tert-butyl 4-[1-(2,6-dioxo-3-piperidyl)-3-ethyl-2-oxo-benzimidazol-5-yl]piperidine-1-carboxylate (B-218, 120 mg, 243.50 μmol, 48% yield) was synthesized from tert-butyl 4-[1-(2,6-dioxo-3-piperidyl)-3-ethyl-2-oxo-benzimidazol-5-yl]-3,6-dihydro-2H-pyridine-1-carboxylate (B-217) in a similar fashion to Compound B-103, except using 0.25 eq. palladium hydroxide (10% on carbon). The material was purified by column chromatography (30-70% ethyl acetate in petroleum ether). LCMS (ES+): m/z 401.1 [M−tBu+H]⁺.

### Step 7: 3-[3-ethyl-2-oxo-5-(4-piperidyl)benzimidazol-1-yl]piperidine-2,6-dione (B-219)

3-[3-ethyl-2-oxo-5-(4-piperidyl)benzimidazol-1-yl]piperidine-2,6-dione (B-219, 120 mg, 232.61 μmol, 88% yield, TFA salt) was synthesized from tert-butyl 4-[1-(2,6-dioxo-3-piperidyl)-3-ethyl-2-oxo-benzimidazol-5-yl]piperidine-1-carboxy late (B-218) in a similar fashion to Compound A-62, except using 5 eq. TFA. LCMS (ES+): m/z 357.2 [M+H]⁺.

### N-[[2-(2,6-dioxo-3-piperidyl)-1-oxo-isoindolin-4-yl]methyl]azetidine-3-carboxamide (B-221)

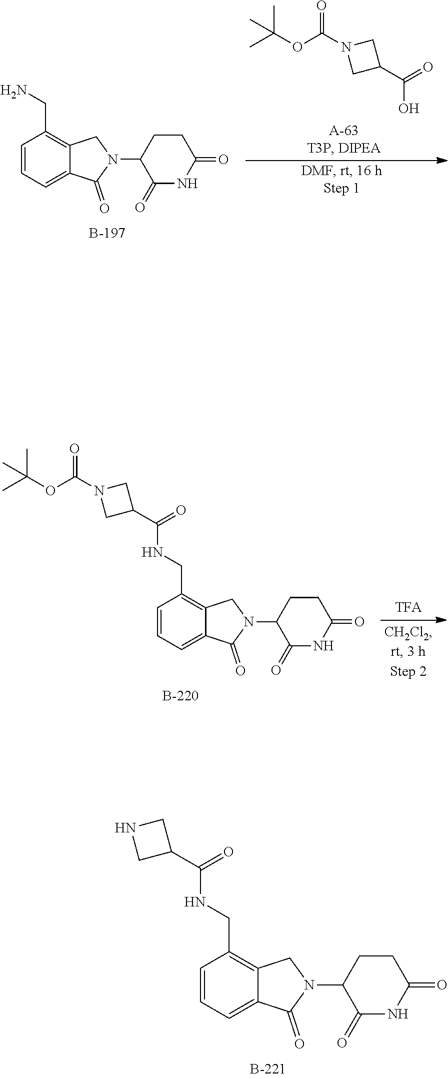 [SMILES: NCc1cccc2c1CN(C1CCC(=O)NC1=O)C2=O ; CC(C)(C)OC(=O)N1CC(C(=O)O)C1 ; CC(C)(C)OC(=O)N1CC(C(=O)NCc2cccc3c2CN(C2CCC(=O)NC2=O)C3=O)C1 ; O=C1CCC(N2Cc3c(CNC(=O)C4CNC4)cccc3C2=O)C(=O)N1]

### Step 1: tert-butyl 3-(((2-(2,6-dioxopiperidin-3-yl)-1-oxoisoindolin-4-yl)methyl)carbamoyl)azetidine-1-carboxylate (B-220)

tert-butyl 3-(((2-(2,6-dioxopiperidin-3-yl)-1-oxoisoindolin-4-yl)methyl)carbamoyl)azetidine-1-carboxy late (B-220, 35 mg, 74.66 μmol, 68% yield) was synthesized from 1-tert-butoxycarbonylazetidine-3-carboxylic acid (A-63) and 3-[4-(aminomethyl)-1-oxo-isoindolin-2-yl]piperidine-2,6-dione (B-197) in a similar fashion to Compound A-88, except using 1 eq. B-197 and 2 eq. 1-propanephosphonic anhydride (50% in ethyl acetate). LCMS (ES+): m/z 357.2 [M−Boc+H]⁺.

### Step 2: N-[[2-(2,6-dioxo-3-piperidyl)-1-oxo-isoindolin-4-yl]methyl]azetidine-3-carboxamide (B-221)

N-[[2-(2,6-dioxo-3-piperidyl)-1-oxo-isoindolin-4-yl]methyl]azetidine-3-carboxamide (B-221, 30 mg, 45.98 μmol, 60% yield, TFA salt) was synthesized from tert-butyl 3-[[2-(2,6-dioxo-3-piperidyl)-1-oxo-isoindolin-4-yl]methylcarbamoyl]azetidine-1-carboxylate (B-220) in a similar fashion to Compound A-62, except using 3 eq. TFA. The material was triturated with diethyl ether. LCMS (ES+): m/z 357.2 [M+H]⁺.

### 2-[[1-[2-[[2-(2,6-dioxo-3-piperidyl)-1-oxo-isoindolin-4-yl]amino]acetyl]-4-piperidyl]oxy]acetic acid (B-224)

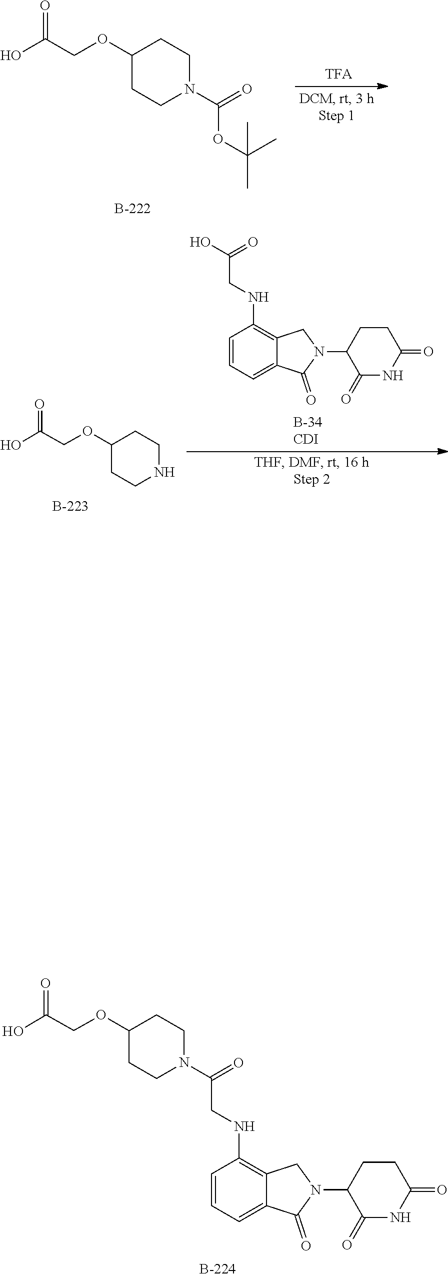 [SMILES: CC(C)(C)OC(=O)N1CCC(OCC(=O)O)CC1 ; O=C(O)CNc1cccc2c1CN(C1CCC(=O)NC1=O)C2=O ; O=C(O)COC1CCNCC1 ; O=C(O)COC1CCN(C(=O)CNc2cccc3c2CN(C2CCC(=O)NC2=O)C3=O)CC1]

### Step 1: 2-(4-piperidyloxy)acetic acid (B-223)

2-(4-piperidyloxy)acetic acid (B-223, 300 mg, 945.44 μmol, 82% yield, TFA salt) was synthesized from 2-[(1-tert-butoxycarbonyl-4-piperidyl)oxy]acetic acid (B-222) in a similar fashion to Compound A-62, except using 5 eq. TFA. The material was triturated with diethyl ether. LCMS (ES+): m/z 160.1 [M+H]⁺.

### Step 2: 2-[[1-[2-[[2-(2,6-dioxo-3-piperidyl)-1-oxo-isoindolin-4-yl]amino]acetyl]-4-piperidyl]oxy]acetic acid (B-224)

2-[[1-[2-[[2-(2,6-dioxo-3-piperidyl)-1-oxo-isoindolin-4-yl]amino]acetyl]-4-piperidyl]oxy]acetic acid (B-224, 60 mg, 101.92 μmol, 22% yield, TFA salt) was synthesized from 2-[[2-(2,6-dioxo-3-piperidyl)-1-oxo-isoindolin-4-yl]amino]acetic acid (B-34) and 2-(4-piperidyloxy)acetic acid (B-223) in a similar fashion to Compound B-117, except using 1.5 eq. CDI and 3 eq. B-223. LCMS (ES+): m/z 459.0 [M+H]⁺.

### 3-[5-(3-aminoprop-1-ynyl)-3-methyl-2-oxo-benzimidazol-1-yl]piperidine-2,6-dione (B-227)

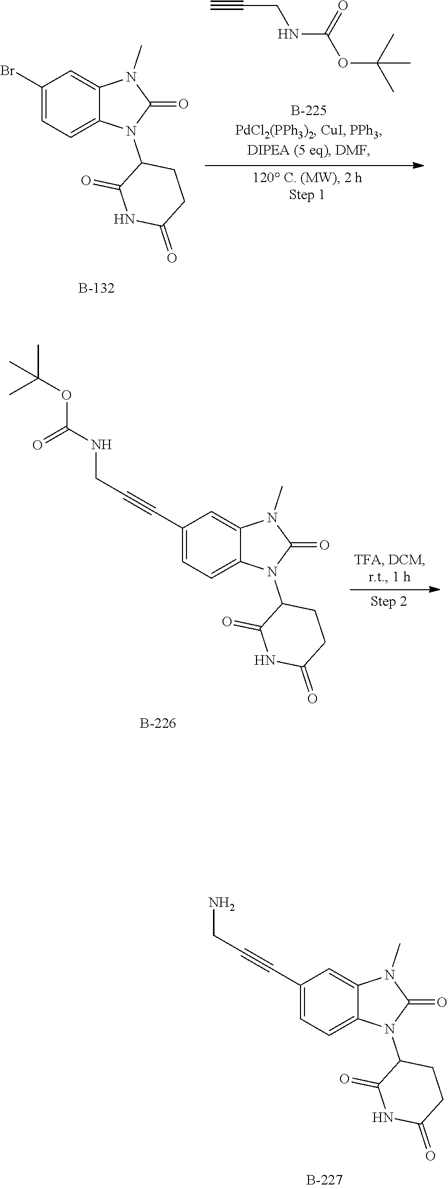 [SMILES: Cn1c(=O)n(C2CCC(=O)NC2=O)c2ccc(Br)cc21 ; C#CCNC(=O)OC(C)(C)C ; Cn1c(=O)n(C2CCC(=O)NC2=O)c2ccc(C#CCNC(=O)OC(C)(C)C)cc21 ; Cn1c(=O)n(C2CCC(=O)NC2=O)c2ccc(C#CCN)cc21]

### Step 1: ter-butyl N-[3-[1-(2,6-dioxo-3-piperidyl)-3-methyl-2-oxo-benzimidazol-5-yl]prop-2-ynyl]carbamate (B-226)

Into a 20 mL microwave vial containing well-stirred solution of 3-(5-bromo-3-methyl-2-oxo-benzimidazol-1-yl)piperidine-2,6-dione (B-132, 150 mg, 443.58 μmol) in anhydrous DMF (5 mL) were added copper (I) iodide (84.48 mg, 443.58 μmol), N-Boc-propargylamine (B-225, 137.68 mg, 887.16 μmol), DIPEA (222.60 mg, 1.72 mmol, 0.3 mL), triphenylphosphine (116.35 mg, 443.58 μmol) and bis(triphenylphosphine) palladium chloride (62.27 mg, 88.72 μmol) at room temperature. The reaction mixture was purged by bubbling nitrogen gas through for 20 min. The vial was sealed and subjected to microwave irradiation at 120° C. for 2 h. The solvent was removed under reduced pressure and the residue was purified by flash silica gel (230-400 mesh) column chromatography (90% Ethyl acetate in petroleum ether) to afford tert-butyl N-[3-[1-(2,6-dioxo-3-piperidyl)-3-methyl-2-oxo-benzimidazol-5-yl]prop-2-ynyl]carbamate (B-226, 70 mg, 120.77 μmol, 27% yield) as an off-white solid. LCMS (ES−): m/z 411.1 [M−H]⁻.

### Step 2: 3-[5-(3-aminoprop-1-ynyl)-3-methyl-2-oxo-benzimidazol-1-yl]piperidine-2,6-dione (B-227)

3-[5-(3-aminoprop-1-ynyl)-3-methyl-2-oxo-benzimidazol-1-yl]piperidine-2,6-dione (B-227, 150 mg, 279.46 μmol, 89% yield, TFA salt) was synthesized from tert-butyl N-[3-[1-(2,6-dioxo-3-piperidyl)-3-methyl-2-oxo-benzimidazol-5-yl]prop-2-ynyl]carbamate (B-226) in a similar fashion to Compound A-62, except using 1.5 eq. TFA. The material was triturated with diethyl ether. LCMS (ES−): m/z 311.1 [M−H]⁻

### 2-[1-[2-[[2-(2,6-dioxo-3-piperidyl)-1-oxo-isoindolin-4-yl]amino]acetyl]azetidin-3-yl]oxyacetic acid (B-230)

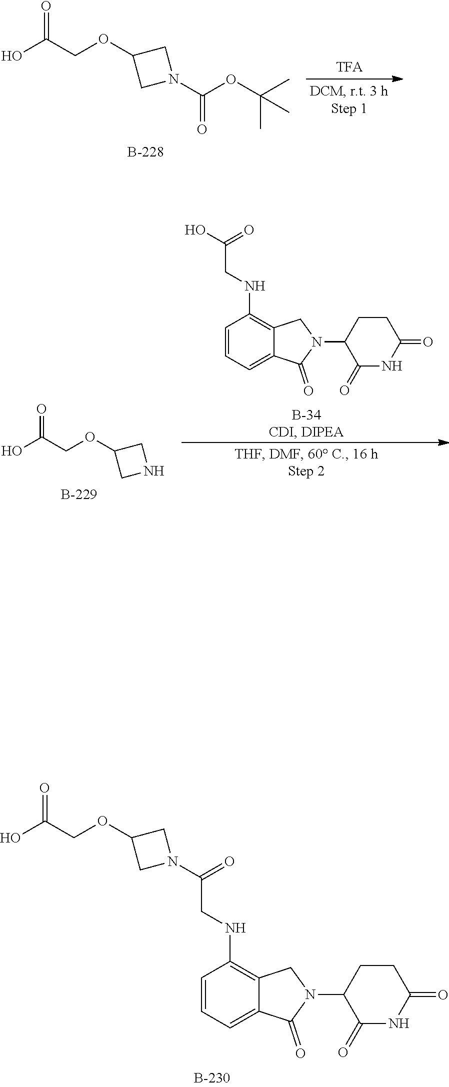

### Step 1: 2-(azetidin-3-yloxy)acetic acid (B-229)

2-(azetidin-3-yloxy)acetic acid (B-229, 680 mg, 2.65 mmol, 88% yield, TFA salt) as was synthesized from 2-(1-tert-butoxycarbonylazetidin-3-yl)oxyacetic acid (B-228) in a similar fashion to Compound A-62, except using 17 eq. TFA. LCMS: ELSD (ES+): m/z 132.1 [M+H]⁺.

### Step 2: 2-[1-[2-[[2-(2,6-dioxo-3-piperidyl)-1-oxo-isoindolin-4-yl]amino]acetyl]azetidin-3-yl]oxyacetic acid (B-230)

2-[1-[2-[[2-(2,6-dioxo-3-piperidyl)-1-oxo-isoindolin-4-yl]amino]acetyl]azetidin-3-yl]oxyacetic acid (B-230, 160 mg, 261.20 μmol, 28% yield, TFA salt) was synthesized from 2-[[2-(2,6-dioxo-3-piperidyl)-1-oxo-isoindolin-4-yl]amino]acetic acid (B-34) and 2-(azetidin-3-yloxy)acetic acid (B-229) in a similar fashion to Compound B-117, except using 2 eq. CDI, 1.3 eq. of B-229, and adding 3 eq. DIPEA. Upon completion, the solvent was removed under reduced pressure and the residue purified by reverse phase Prep-HPLC [Purification method: X-BRIDGE (C8 19×150 mm), 5 μM; Mobile phase A: 0.1% TFA in water; Mobile phase B: MeCN]. LCMS (ES+): m/z 431.1 [M+H]⁺.

### 1-[2-[2-(2,6-dioxo-3-piperidyl)-1-oxo-isoindolin-4-yl]oxyacetyl]-N-[2-fluoro-5-[(4-oxo-1-piperidyl)sulfonylmethyl]phenyl]azetidine-3-carboxamide (B-231)

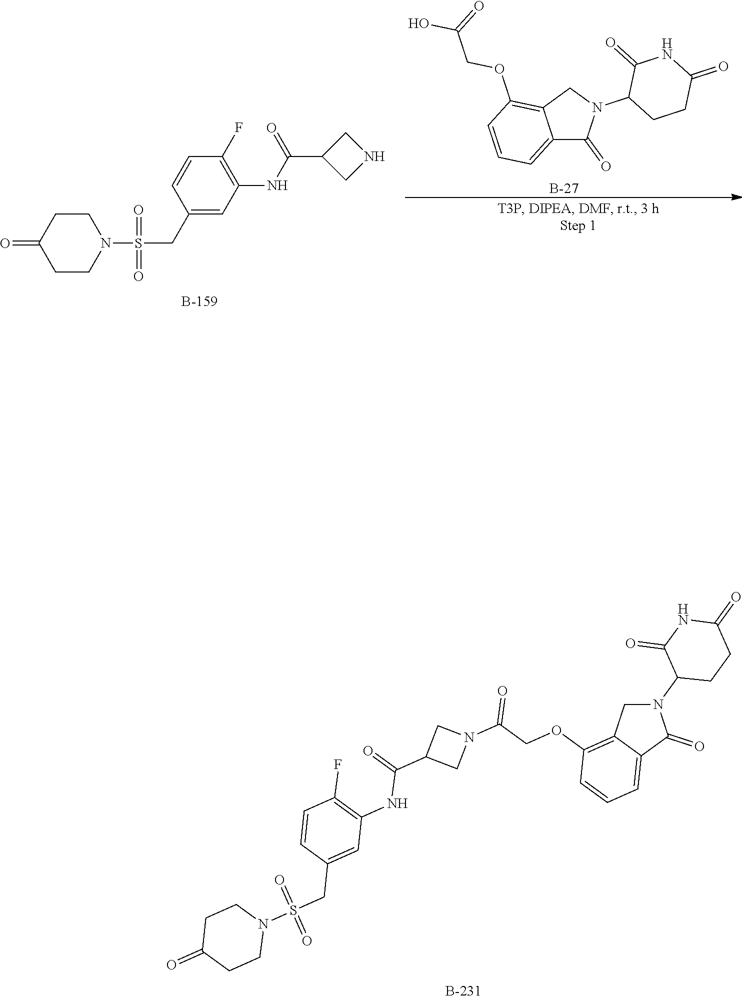

### Step 1: 1-[2-[2-(2,6-dioxo-3-piperidyl)-1-oxo-isoindolin-4-yl]oxyacetyl]-N-[2-fluoro-5-[(4-oxo-1-piperidyl)sulfonylmethyl]phenyl]azetidine-3-carboxamide (B-231)

1-[2-[2-(2,6-dioxo-3-piperidyl)-1-oxo-isoindolin-4-yl]oxyacetyl]-N-[2-fluoro-5-[(4-oxo-1-piperidyl)sulfonylmethyl]phenyl]azetidine-3-carboxamide (B-231, 80 mg, 83.72 μmol, 44% yield) was synthesized from N-[2-fluoro-5-[(4-oxo-1-piperidyl)sulfonylmethyl]phenyl]azetidine-3-carboxamide (B-159) and 2-[2-(2,6-dioxo-3-piperidyl)-1-oxo-isoindolin-4-yl]oxyacetic acid (B-27) in a similar fashion to Compound A-88, except using 1.3 eq. B-27 and 3 eq. 1-propanephosphonic anhydride (50% in ethyl acetate). Upon completion, the solvent was removed under reduced pressure and the residue suspended in water. The resulting precipitate was filtered and dried under reduced pressure. The material was used in the next step without further purification. LCMS (ES+): m/z 670.0 [M+H]⁺.

### 3-[5-(4-piperidyl) indolin-1-yl]piperidine-2,6-dione (B-236)

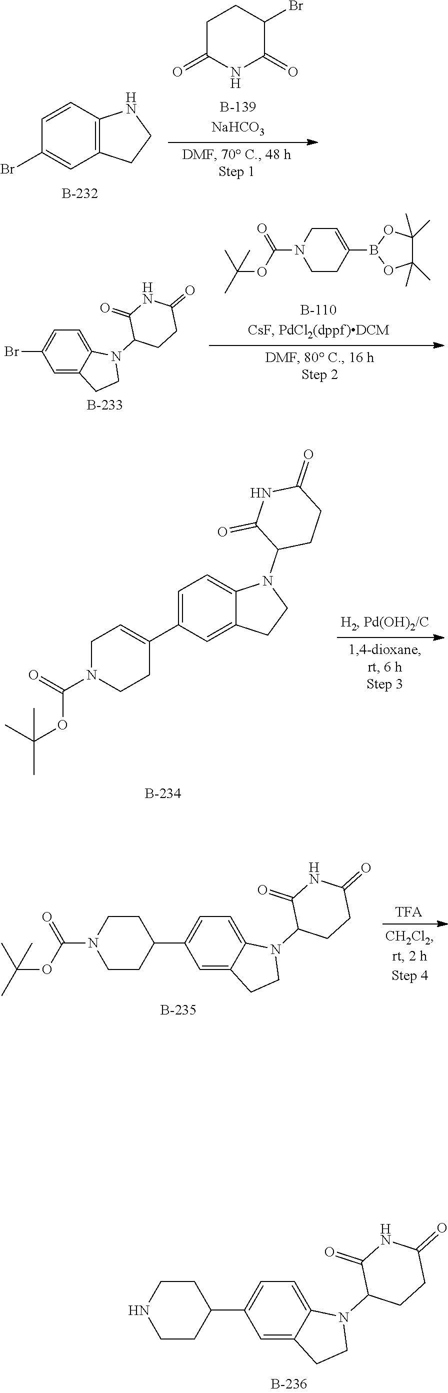

### Step 1: 3-(5-bromoindolin-1-yl)piperidine-2,6-dione (B-233)

3-(5-bromoindolin-1-yl)piperidine-2,6-dione (B-233, 1.02 g, 3.24 mmol, 32% yield) was synthesized from 5-bromoindoline (B-232) and 3-bromopiperidine-2,6-dione (B-139) in a similar fashion to Compound A-4, except using 3 eq. sodium bicarbonate and heating to 70° C. for 48 h. The aqueous layer was extracted with ethyl acetate instead of diethyl ether. ¹H NMR (400 MHz, DMSO-d₆) δ 10.81 (s, 1H), 7.14 (s, 1H), 7.08 (d, J=8.32 Hz, 1H), 6.43 (d, J=8.4 Hz, 1H), 4.65-4.61 (m, 1H), 3.48-3.42 (m, 1H), 3.28-3.13 (m, 1H), 3.01-2.89 (m, 2H), 2.81-2.72 (m, 1H), 2.59-2.56 (m, 1H), 2.25-2.14 (m, 1H), 2.13-1.91 (m, 1H).

### Step 2: tert-butyl 4-[1-(2,6-dioxo-3-piperidyl) indolin-5-yl]-3,6-dihydro-2H-pyridine-1-carboxylate (B-234)

tert-butyl 4-[1-(2,6-dioxo-3-piperidyl) indolin-5-yl]-3,6-dihydro-2H-pyridine-1-carboxylate (B-234, 80 mg, 169.14 μmol, 35% yield) was synthesized from 3-(5-bromoindolin-1-yl)piperidine-2,6-dione (B-233) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 1.5 eq. B-110, 2 eq. of CsF and 0.1 eq. [1,1′-Bis(diphenylphosphino)ferrocene]dichloropalladium(II) complex with dichloromethane. The reaction mixture was heated at 80° C. Upon completion, the reaction mixture was filtered through Celite, washing with Ethyl acetate. The filtrate was concentrated and the residue purified by flash silica gel (60-120 mesh) column chromatography (40-50% Ethyl acetate/petroleum ether). LCMS (ES+): m/z 412.1 [M+H]⁺.

### Step 3: tert-butyl 4-[1-(2,6-dioxo-3-piperidyl) indolin-5-yl]piperidine-1-carboxylate (B-235)

tert-butyl 4-[1-(2,6-dioxo-3-piperidyl) indolin-5-yl]piperidine-1-carboxy late (B-235, 35 mg, 69.41 μmol, 99% yield) was synthesized from tert-butyl 4-[1-(2,6-dioxo-3-piperidyl) indolin-5-yl]-3,6-dihydro-2H-pyridine-1-carboxylate (B-234) in a similar fashion to Compound B-103, except using 0.2 eq. palladium hydroxide (10% on carbon) added in two portions. LCMS (ES+): m/z 358.2 [M−tBu+H]⁺.

### Step 4: 3-[5-(4-piperidyl) indolin-1-yl]piperidine-2,6-dione (B-236)

3-[5-(4-piperidyl) indolin-1-yl]piperidine-2,6-dione (B-236, 30 mg, 65.98 μmol, 95% yield, TFA salt) was synthesized from tert-butyl 4-[1-(2,6-dioxo-3-piperidyl) indolin-5-yl]piperidine-1-carboxylate (B-235) in a similar fashion to Compound A-62, except using 3 eq. TFA. The material was triturated with diethyl ether. LCMS (ES+): m/z 314.1 [M+H]⁺.

### 3-[2-methyl-S-(4-piperidyl) indolin-1-yl]piperidine-2,6-dione (B-241)

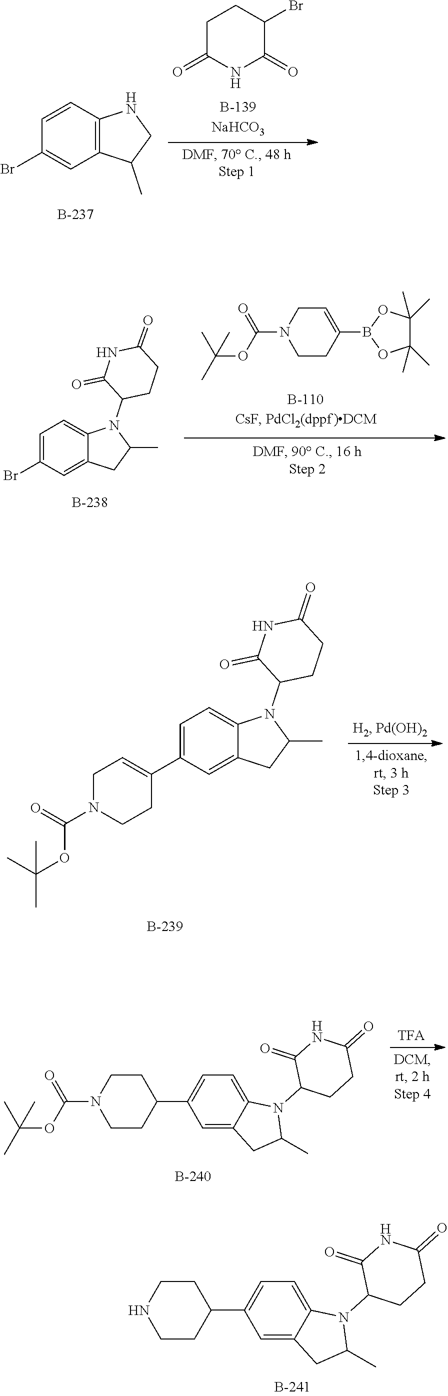

### Step 1: 3-(5-bromo-2-methyl-indolin-1-yl)piperidine-2,6-dione (B-238)

3-(5-bromo-2-methyl-indolin-1-yl)piperidine-2,6-dione (B-238, 800 mg, 2.47 mmol, 29% yield) was synthesized from 5-bromo-2-methyl-indoline (B-237) and 3-bromopiperidine-2,6-dione (B-139) in a similar fashion to Compound A-4, except using 3 eq. sodium bicarbonate and heating at 70° C. for 48 h. The aqueous layer was extracted with ethyl acetate instead of diethyl ether. ¹H NMR (400 MHz, DMSO-d₆) δ 10.82 (s, 1H), 7.11-7.10 (m, 1H), 7.05-7.00 (m, 1H), 6.17 (d, J=8.4 Hz, 1H), 4.47-4.36 (m, 1H), 3.83-3.80 (m, 1H), 3.25-3.11 (m, 1H), 2.78-2.70 (m, 1H), 2.58-2.53 (m, 2H), 2.23-2.20 (m, 1H), 1.90-1.88 (m 1H), 1.21-1.20 (m, 3H).

### Step 2: tert-butyl 4-[1-(2,6-dioxo-3-piperidyl)-2-methyl-indolin-5-yl]-3,6-dihydro-2H-pyridine-1-carboxylate (B-239)

tert-butyl 4-[1-(2,6-dioxo-3-piperidyl)-2-methyl-indolin-5-yl]-3,6-dihydro-2H-pyridine-1-carboxylate (B-239, 53.33 mg, 113.18 μmol, 35% yield) was synthesized from 3-(5-bromo-2-methyl-indolin-1-yl)piperidine-2,6-dione (B-238) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 2 eq. B-110 and 0.2 eq. [1,1′-Bis(diphenylphosphino)ferrocene]dichloropalladium(II) complex with dichloromethane. Upon completion, the mixture was diluted with Ethyl acetate and filtered through Celite washing with Ethyl acetate. The filtrate was concentrated under reduced pressure and the residue purified by flash silica gel column chromatography (28-30% Ethyl acetate in petroleum ether). LCMS (ES+): m/z 426.2 [M+H]⁺.

### Step 3: tert-butyl 4-[1-(2,6-dioxo-3-piperidyl)-2-methyl-indolin-5-yl]piperidine-1-carboxylate (B-240)

tert-butyl 4-[1-(2,6-dioxo-3-piperidyl)-2-methyl-indolin-5-yl]piperidine-1-carboxylate (B-240, 48 mg, 103.85 μmol, 88% yield) was synthesized from tert-butyl 4-[1-(2,6-dioxo-3-piperidyl)-2-methyl-indolin-5-yl]-3,6-dihydro-2H-pyridine-1-carboxylate (B-239) in a similar fashion to Compound B-103, except using 0.1 eq. palladium hydroxide (10% on carbon). LCMS (ES+): m/z 428.3 [M+H]⁺.

### Step 4: 3-[2-methyl-5-(4-piperidyl) indolin-1-yl]piperidine-2,6-dione (B-241)

3-[2-methyl-5-(4-piperidyl) indolin-1-yl]piperidine-2,6-dione (B-241, 40 mg, 89.07 μmol, 84% yield, TFA salt) was synthesized from tert-butyl 4-[1-(2,6-dioxo-3-piperidyl)-2-methyl-indolin-5-yl]piperidine-1-carboxylate (B-240) in a similar fashion to Compound A-62, except using 62 eq. TFA. The material was triturated with diethyl ether. LCMS (ES+): m/z 328.3 [M+H]⁺.

### 4-[1-(2,6-dioxo-3-piperidyl)-3-methyl-2-oxo-benzimidazol-5-yl]-N-[2-fluoro-3-[(4-oxo-1-piperidyl)sulfonylmethyl]phenyl]piperidine-1-carboxamide (B-242)

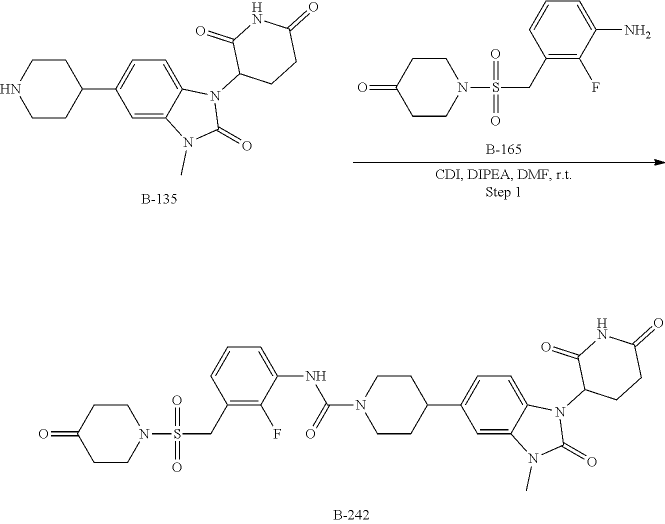 [SMILES: Cn1c(=O)n(C2CCC(=O)NC2=O)c2ccc(C3CCNCC3)cc21 ; Nc1cccc(CS(=O)(=O)N2CCC(=O)CC2)c1F ; Cn1c(=O)n(C2CCC(=O)NC2=O)c2ccc(C3CCN(C(=O)Nc4cccc(CS(=O)(=O)N5CCC(=O)CC5)c4F)CC3)cc21]

### Step 1: 4-[1-(2,6-dioxo-3-piperidyl)-3-methyl-2-oxo-benzimidazol-5-yl]-N-[2-fluoro-3-[(4-oxo-1-piperidyl)sulfonylmethyl]phenyl]piperidine-1-carboxamide (B-242)

Into a 10 mL single neck round bottom flask containing a well-stirred solution of 1-[(3-amino-2-fluoro-phenyl)methylsulfonyl]piperidin-4-one (B-165, 50 mg, 0.174 mmol) in a mixture of 1:1 anhydrous DCM/DMF (1 mL) were added DIPEA (67.71 mg, 0.523 mmol) and CDI (42.47 mg, 0.261 mmol). The reaction mixture was stirred at ambient temperature for 4 h. Subsequently, 3-[3-methyl-2-oxo-5-(4-piperidyl)benzimidazol-1-yl]piperidine-2,6-dione (B-135, 79.70 mg, 0.174 mmol, TFA salt) in anhydrous DMF (0.5 mL) was added, and the resulting solution was stirred for 16 h. The reaction mixture was concentrated under reduced pressure and the residue was purified by reverse phase HPLC following the method: Column XSelect (150×19 mm, 5 μm) with Solvent A: 0.1% TFA in water; Solvent B: Acetonitrile; Flow rate: 15 mL/min; RT=12 min to afford 4-[1-(2,6-dioxo-3-piperidyl)-3-methyl-2-oxo-benzimidazol-5-yl]-N-[2-fluoro-3-[(4-oxo-1-piperidyl)sulfonylmethyl]phenyl]piperidine-1-carboxamide (B-242, 20 mg, 0.019 mmol, 11% yield) as an off-white solid. LCMS (ESI): m/z 654.8 [M+H]⁺.

### 3-[5-(2,7-diazaspiro[3.5]nonan-7-yl)-3-methyl-2-oxo-benzimidazol-1-yl]piperidine-2,6-dione (B-246)

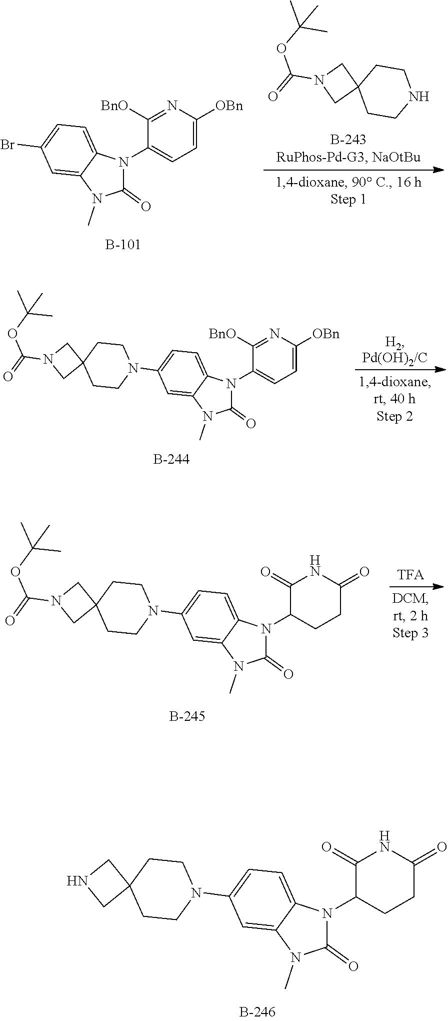 [SMILES: Cn1c(=O)n(-c2ccc(OCc3ccccc3)nc2OCc2ccccc2)c2ccc(Br)cc21 ; CC(C)(C)OC(=O)N1CC2(CCNCC2)C1 ; Cn1c(=O)n(-c2ccc(OCc3ccccc3)nc2OCc2ccccc2)c2ccc(N3CCC4(CC3)CN(C(=O)OC(C)(C)C)C4)cc21 ; "H2, Pd(OH)2/C" ; Cn1c(=O)n(C2CCC(=O)NC2=O)c2ccc(N3CCC4(CC3)CN(C(=O)OC(C)(C)C)C4)cc21 ; Cn1c(=O)n(C2CCC(=O)NC2=O)c2ccc(N3CCC4(CC3)CNC4)cc21]

### Step 1: tert-butyl 7-[1-(2,6-dibenzyloxy-3-pyridyl)-3-methyl-2-oxo-benzimidazol-5-yl]-2,7-diazaspiro[3.5]nonane-2-carboxylate (B-244)

Into a 25 mL pressure tube containing a well-stirred solution of tert-butyl 2,7-diazaspiro[3.5]nonane-2-carboxylate (B-243, 197.22 mg, 871.44 μmol) and 5-bromo-1-(2,6-dibenzyloxy-3-pyridyl)-3-methyl-benzimidazol-2-one (B-101, 300 mg, 580.96 μmol) in 1,4-dioxane (3 mL) was added sodium tert-butoxide (167.49 mg, 1.74 mmol). The reaction mixture was purged by bubbling nitrogen gas through for 5 min. then RuPhos-Pd-G3 (0.1 g, 119.42 μmol) was added and degassed for another 5 min. The tube was sealed and the reaction mixture was heated to 90° C. After 16 h the mixture was cooled to room temperature and diluted with Ethyl acetate (50 mL) and washed with water (3×40 mL). The Ethyl acetate layer was dried over anhydrous sodium sulfate and solvent was removed under reduced pressure to afford the crude product that was purified by flash silica gel column chromatography (80% Ethyl acetate in petroleum ether) to afford tert-butyl 7-[1-(2,6-dibenzyloxy-3-pyridyl)-3-methyl-2-oxo-benzimidazol-5-yl]-2,7-diazaspiro[3.5]nonane-2-carboxylate (B-244, 150 mg, 206.26 μmol, 36% yield) as a pale yellow solid. LCMS (ES+): m/z 662.3 [M+H]⁺.

### Step 2: tert-butyl 7-[1-(2,6-dioxo-3-piperidyl)-3-methyl-2-oxo-benzimidazol-5-yl]-2,7-diazaspiro[3.5]nonane-2-carboxylate (B-245)

tert-butyl 7-[1-(2,6-dioxo-3-piperidyl)-3-methyl-2-oxo-benzimidazol-5-yl]-2,7-diazaspiro[3.5]nonane-2-carboxylate (B-245, 100 mg, 136.07 μmol, 64% yield, 66% purity) was synthesized from tert-butyl 7-[1-(2,6-dibenzyloxy-3-pyridyl)-3-methyl-2-oxo-benzimidazol-5-yl]-2,7-diazaspiro[3.5]nonane-2-carboxylate (B-244) in a similar fashion to Compound B-103, except using 0.2 eq. palladium hydroxide on carbon (20% by weight, 50% water). LCMS (ES+): m/z 484.1 [M+H]⁺.

### Step 3: 3-[5-(2,7-diazaspiro[3.5]nonan-7-yl)-3-methyl-2-oxo-benzimidazol-1-yl]piperidine-2,6-dione (B-246)

3-[5-(2,7-diazaspiro[3.5]nonan-7-yl)-3-methyl-2-oxo-benzimidazol-1-yl]piperidine-2,6-dione (B-246, 85 mg, 152.92 μmol, 78% yield, TFA salt) was synthesized from tert-butyl 7-[1-(2,6-dioxo-3-piperidyl)-3-methyl-2-oxo-benzimidazol-5-yl]-2,7-diazaspiro[3.5]nonane-2-carboxylate (B-245) in a similar fashion to Compound A-62, except using 5 eq. TFA. The material was triturated with diethyl ether. LCMS (ES+): m/z 384.3 [M+H]⁺.

### 4-[1-(2,6-dioxo-3-piperidyl)-3-methyl-2-oxo-benzimidazol-5-yl]-N-[2-fluoro-5-[(4-oxo-1-piperidyl)sulfonylmethyl]phenyl]piperidine-1-carboxamide (B-247)

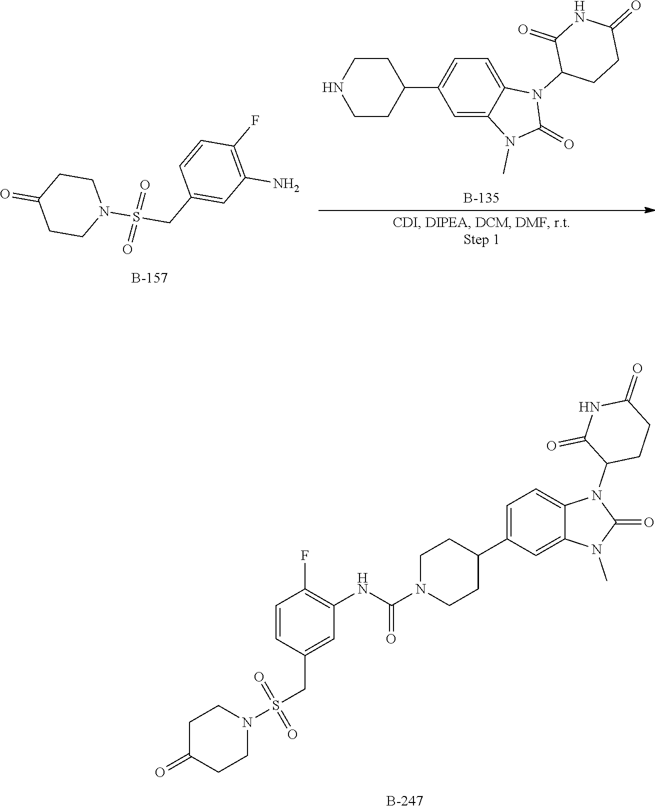 [SMILES: Nc1cc(CS(=O)(=O)N2CCC(=O)CC2)ccc1F ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)Nc4cc(CS(=O)(=O)N5CCC(=O)CC5)ccc4F)CC3)cc21]

### Step 1: 4-[1-(2,6-dioxo-3-piperidyl)-3-methyl-2-oxo-benzimidazol-5-yl]-N-[2-fluoro-5-[(4-oxo-1-piperidyl)sulfonylmethyl]phenyl]piperidine-1-carboxamide (B-247)

4-[1-(2,6-dioxo-3-piperidyl)-3-methyl-2-oxo-benzimidazol-5-yl]-N-[2-fluoro-5-[(4-oxo-1-piperidyl)sulfonylmethyl]phenyl]piperidine-1-carboxamide (B-247, 40 mg, 0.032 mmol, 9% yield) was synthesized from 1-[(3-amino-4-fluoro-phenyl)methylsulfonyl]piperidin-4-one (B-157) and 3-[3-methyl-2-oxo-5-(4-piperidyl)benzimidazol-1-yl]piperidine-2,6-dione (B-135) in a similar fashion to Compound B-242. LCMS (ESI): m/z 655.1 [M+H]⁺.

### 3-[5-(4-hydroxy-1-piperidyl)-3-methyl-2-oxo-benzimidazol-1-yl]piperidine-2,6-dione (B-251)

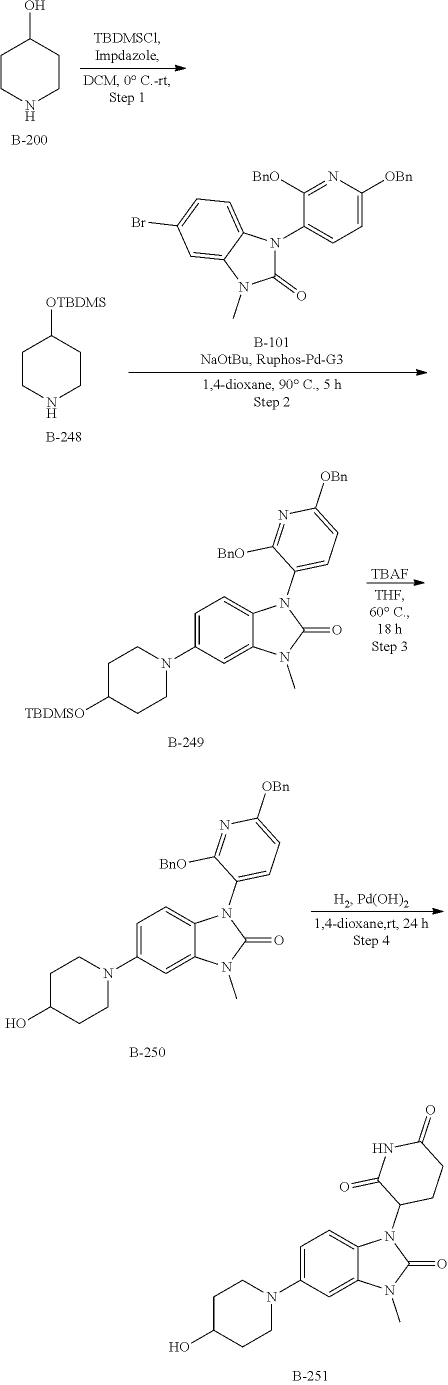

### Step 1: tert-butyl-dimethyl-(4-piperidyloxy) silane (B-248)

Into a 250 mL two neck round bottom flask containing a well-stirred suspension of piperidin-4-ol (B-200, 5 g, 49.43 mmol) in DCM (100 mL) was added imidazole (6.73 g, 98.87 mmol) at room temperature. The reaction mixture was cooled to 0° C. and tert-butyldimethylsilyl chloride (8.20 g, 54.38 mmol, 10.12 mL) was added. The reaction mixture was allowed to stir at ambient temperature for 16 h. The reaction mixture was extracted with dichloromethane (2×300 mL), washed with water (250 mL) and brine (200 mL). The combined organics were dried over sodium sulfate, filtered and the solvent removed under reduced pressure to afford tert-butyl-dimethyl-(4-piperidyloxy) silane (B-248, 4 g, 11.77 mmol, 24% yield) as a pale yellow colored liquid. The material was used in the next step without further purification. LCMS (ES+): m/z 216.2 [M+H]⁺.

### Step 2: 5-[4-[tert-butyl(dimethyl)silyl]oxy-1-piperidyl]-1-(2,6-dibenzyloxy-3-pyridyl)-3-methyl-benzimidazol-2-one (B-249)

5-[4-[tert-butyl(dimethyl)silyl]oxy-1-piperidyl]-1-(2,6-dibenzyloxy-3-pyridyl)-3-methyl-benzimidazol-2-one (B-249, 600 mg, 862.83 μmol, 56% yield) was synthesized from 5-bromo-1-(2,6-dibenzyloxy-3-pyridyl)-3-methyl-benzimidazol-2-one (B-101) and tert-butyl-dimethyl-(4-piperidyloxy) silane (B-248) in a similar fashion to Compound B-244, except using 1.1 eq. B-248 and 0.15 eq. RuPhos-Pd-G3. LCMS (ES+): m/z 651.3 [M+H]⁺.

### Step 3: 1-(2,6-dibenzyloxy-3-pyridyl)-5-(4-hydroxy-1-piperidyl)-3-methyl-benzimidazol-2-one (B-250)

Into a 25 mL pressure tube containing a well-stirred solution of 5-[4-[tert-butyl(dimethyl)silyl]oxy-1-piperidyl]-1-(2,6-dibenzyloxy-3-pyridyl)-3-methyl-benzimidazol-2-one (B-249, 600 mg, 921.83 μmol) in THF (4 mL) was added tetrabutylammonium fluoride (1 M in THF) (921.83 μmol, 1 mL) at room temperature. The tube was sealed, and the reaction was heated to 60° C. for 18 h. The mixture was cooled to room temperature and poured into water (40 mL) and extracted with DCM (2×60 mL). The organic layer was dried over anhydrous sodium sulfate, filtered and solvent removed under reduced pressure. The residue was purified by flash silica gel column chromatography (80-100% Ethyl acetate in petroleum ether) to afford 1-(2,6-dibenzyloxy-3-pyridyl)-5-(4-hydroxy-1-piperidyl)-3-methyl-benzimidazol-2-one (B-250, 370 mg, 667.44 μmol, 72% yield) as an off-white solid. LCMS (ES+): m/z 537.2 [M+H]⁺.

### Step 4: 3-[5-(4-hydroxy-1-piperidyl)-3-methyl-2-oxo-benzimidazol-1-yl]piperidine-2,6-dione (B-251)

3-[5-(4-hydroxy-1-piperidyl)-3-methyl-2-oxo-benzimidazol-1-yl]piperidine-2,6-dione (B-251, 50 mg, 100.17 μmol, 13% yield) was synthesized from 1-(2,6-dibenzyloxy-3-pyridyl)-5-(4-hydroxy-1-piperidyl)-3-methyl-benzimidazol-2-one (B-250) in a similar fashion to Compound B-103, except using 0.2 eq. palladium hydroxide on carbon (20% by weight, 50% water). The Celite pad was washed with 1,4-dioxane and DCM. The material was purified by reverse phase prep HPLC (Column: X-Bridge C18, 150×19 mm, 5 μm; Mobile phase A: 10 mM ammonium bicarbonate; Mobile phase B: MeCN). LCMS (ES+): m/z 359.2 [M+H]⁺.

### 2-[[1-[1-(2,6-dioxo-3-piperidyl)-3-methyl-2-oxo-benzimidazol-5-yl]-4-piperidyl]oxy]acetic acid (B-254)

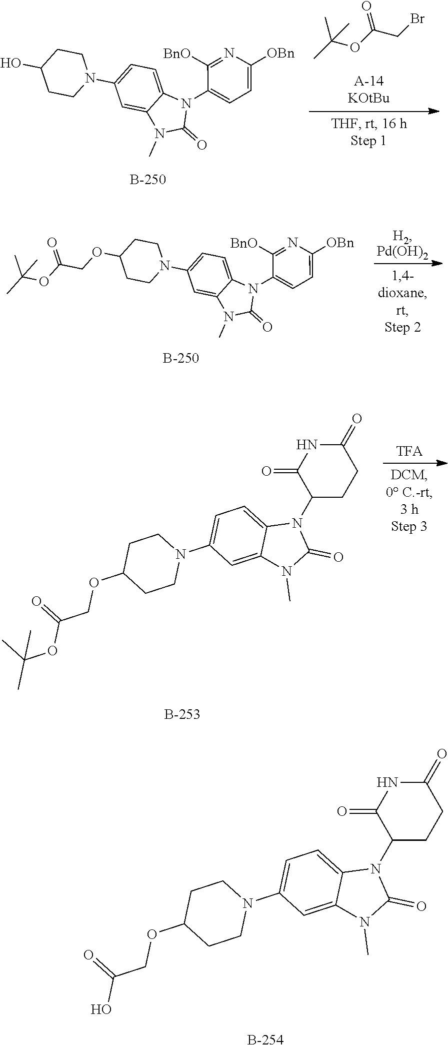

### Step 1: tert-butyl 2-[1-[1-(2,6-dibenzyloxy-3-pyridyl)-3-methyl-2-oxo-benzimidazol-5-yl]-4-piperidyl]oxy]acetate (B-252)

Into a 50 mL single neck round bottom flask containing a well-stirred solution of 1-(2,6-dibenzyloxy-3-pyridyl)-5-(4-hydroxy-1-piperidyl)-3-methyl-benzimidazol-2-one (B-250, 310 mg, 577.69 μmol) in anhydrous THF (10 mL) were added potassium tert-butoxide (194.47 mg, 1.73 mmol) and tert-butyl 2-bromoacetate (A-14, 123.95 mg, 635.46 μmol, 93.19 μL), The reaction mixture was stirred at room temperature for 16 h. The reaction mixture was diluted with Ethyl acetate (40 mL) and washed with water (3×30 mL). The organic layer was dried over anhydrous sodium sulfate and solvent was removed under reduced pressure. The residue was purified by flash silica gel column chromatography (50% Ethyl acetate in petroleum ether) to obtain tert-butyl 2-[[1-[1-(2,6-dibenzyloxy-3-pyridyl)-3-methyl-2-oxo-benzimidazol-5-yl]-4-piperidyl]oxy]acetate (B-252,185 mg, 252.58 μmol, 44% yield) as a pale yellow solid. LCMS (ES+): m/z 651.2 [M+H]⁺.

### Step 2: tert-butyl 2-[[1-[1-(2,6-dioxo-3-piperidyl)-3-methyl-2-oxo-benzimidazol-5-yl]-4-piperidyl]oxy]acetate (B-253)

tert-butyl 2-[1-[1-(2,6-dioxo-3-piperidyl)-3-methyl-2-oxo-benzimidazol-5-yl]-4-piperidyl]oxy]acetate (B-253, 100 mg, 150.36 μmol, 53% yield) was synthesized from tert-butyl 2-[[1-[1-(2,6-dibenzyloxy-3-pyridyl)-3-methyl-2-oxo-benzimidazol-5-yl]-4-piperidyl]oxy]acetate (B-252) in a similar fashion to Compound B-103, except using 0.2 eq. palladium hydroxide on carbon (20% by weight, 50% water). The Celite pad was washed with 1,4-dioxane and DCM. LCMS (ES+): m/z 473.0 [M+H]⁺.

### Step 3: 2-1/1-[1-(2,6-dioxo-3-piperidyl)-3-methyl-2-oxo-benzimidazol-5-yl]-4-piperidyl]oxy]acetic acid (B-254)

2-[[1-[1-(2,6-dioxo-3-piperidyl)-3-methyl-2-oxo-benzimidazol-5-yl]-4-piperidyl]oxy]acetic acid (B-254, 70 mg, 88.51 μmol, 42% yield, TFA salt) was synthesized from tert-butyl 2-[1-[1-(2,6-dioxo-3-piperidyl)-3-methyl-2-oxo-benzimidazol-5-yl]-4-piperidyl]oxy]acetate (B-253) in a similar fashion to Compound A-26, except using 31 eq. TFA. LCMS (ES+): m/z 416.9 [M+H]⁺.

### 3-[5-(azetidin-3-yl)-3-methyl-2-oxo-benzimidazol-1-yl]piperidine-2,6-dione (B-261)

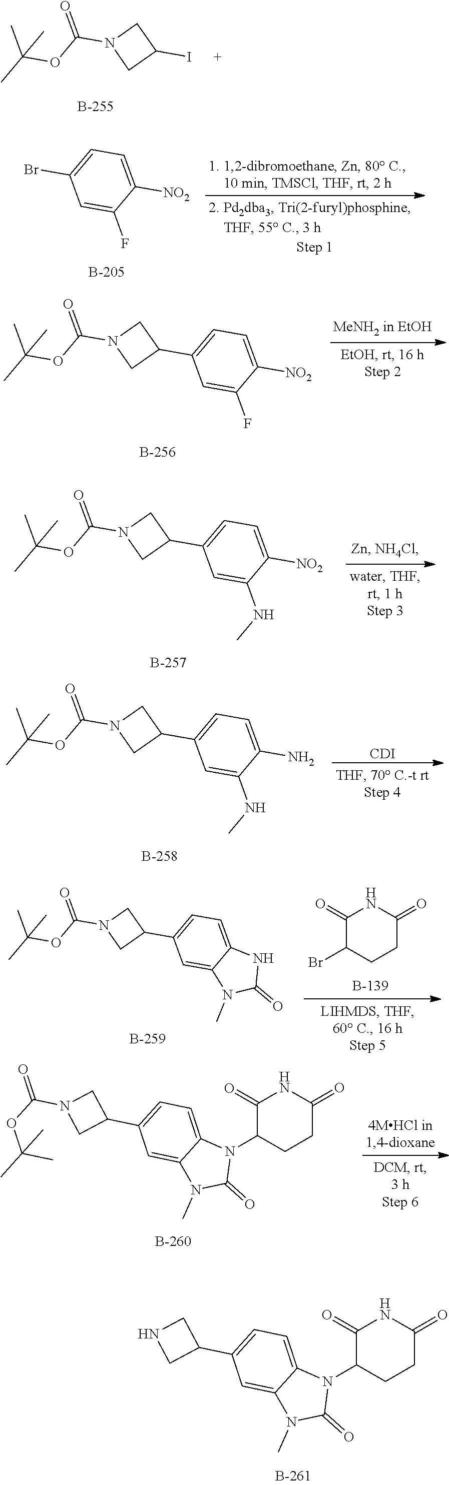

### Step 1: tert-butyl 3-(3-fluoro-4-nitrophenyl)azetidine-1-carboxylate (B-256)

In a 50 mL two neck round bottom flask, 1,2-dibromoethane (398.14 mg, 2.12 mmol, 182.63 μL) was added to a vigorously stirred suspension of zinc (1.20 g, 18.37 mmol) in THF (50 mL) under a nitrogen atmosphere and the resulting mixture was heated at 80° C. for 10 min. Then, trimethylsilylchloride (230.25 mg, 2.12 mmol, 268.98 μL) in THF (50 mL) was added at room temperature, and after stirring for 4 min, a solution of tert-butyl 3-iodoazetidine-1-carboxylate (B-255, 1.4 g, 14.13 mmol) in THF (50 mL) was added dropwise over a period of 15 min. The resulting mixture was stirred at room temperature for 2 h (light grey solid was formed), then Pd₂(dba)₃(0) (129.38 mg, 141.29 μmol) and tri-2-furanylphosphine (196.82 mg, 847.74 μmol) were added, followed by 4-bromo-2-fluoro-1-nitrobenzene (B-205, 3.11 g, 14.13 mmol) in THF (50 mL). The resulting mixture was heated at 55° C. for 3 h. The reaction mixture was quenched with brine solution (50 mL). The aqueous phase was extracted with DCM (2×100 mL), and the combined organic layers were dried over anhydrous sodium sulfate, filtered and concentrated under reduced pressure. The residue was purified using flash silica gel column chromatography (10-40% Ethyl acetate in petroleum ether) to afford tert-butyl 3-(3-fluoro-4-nitrophenyl)azetidine-1-carboxylate (B-256, 2.5 g, 7.20 mmol, 51% yield) as an orange solid. MS (ES+): m/z 197.1 [M−Boc+H]⁺.

### Step 2: tert-butyl 3-[3-(methylamino)-4-nitro-phenyl]azetidine-1-carboxylate (B-257)

Into a 50 mL pressure-tube containing a well-stirred solution of tert-butyl 3-(3-fluoro-4-nitrophenyl)azetidine-1-carboxylate (B-256, 2.4 g, 8.10 mmol) in ethanol (2 mL) was added methylamine solution (33 wt. % in absolute ethanol) (503.13 mg, 16.20 mmol, 559.65 μL). After 16 h, the reaction mixture was diluted with ice cold water (50 mL) and the solid was filtered, washed with water (50 mL) and diethyl ether (50 mL) to obtain tert-butyl 3-[3-(methylamino)-4-nitro-phenyl]azetidine-1-carboxy late (B-257, 1.9 g, 5.87 mmol, 72.47% yield) as yellow solid. The material was used in the next step without further purification. MS (ES+): m/z 252.2 [M−tBu+H]⁺.

### Step 3: tert-butyl 3-(4-amino-3-(methylamino)phenyl)azetidine-1-carboxylate (B-258)

In to a 50 mL single neck round bottom flask containing a stirred solution of tert-buty 13-[3-(methylamino)-4-nitro-phenyl]azetidine-1-carboxylate (B-257, 1.9 g, 6.18 mmol) in THF (10 mL) and water (10 mL) was added zinc powder (325 mesh, 2.02 g, 30.91 mmol). The suspension was cooled to 0° C. and ammonium chloride (1.65 g, 30.91 mmol) added. The reaction mixture was stirred at room temperature for 1 h. The mixture was diluted with THE (10 mL) and filtered through Celite. The filtrate was concentrated under reduced pressure and diluted with DCM (50 mL). The organic layer was washed with sodium bicarbonate solution (50 mL), followed by water (50 mL) and brine solution (50 mL). The organic layer was dried over anhydrous sodium sulfate, filtered and the solvent removed under reduced pressure to afford tert-butyl 3-(4-amino-3-(methyl amino)phenyl)azetidine-1-carboxylate (B-258, 1.5 g, 4.67 mmol, 76% yield) as a brown semi-solid. The material was used in the next step without further purification. MS (ES+): m/z 222.1 [M−tBu+H]⁺.

### Step 4: tert-butyl 3-(3-methyl-2-oxo-2,3-dihydro-1H-benzo[d]imidazol-5-yl)azetidine-1-carboxylate (B-259)

tert-butyl 3-(3-methyl-2-oxo-2,3-dihydro-1H-benzo[d]imidazol-5-yl)azetidine-1-carboxylate (B-259, 0.9 g, 2.14 mmol, 40% yield, 72% purity) was synthesized from tert-butyl 3-(4-amino-3-(methylamino)phenyl)azetidine-1-carboxylate (B-258) in a similar fashion to Compound B-208. Upon completion, the reaction mixture was concentrated under reduced pressure and the residue purified by flash silica gel column chromatography (0-100% Ethyl acetate in petroleum ether). MS (ES+): m/z 304.0 [M+H]⁺.

### Step 5: tert-butyl 3-(1-(2,6-dioxopiperidin-3-yl)-3-methyl-2-oxo-2,3-dihydro-1H-benzo[d]imidazol-5-yl))azetidine-1-carboxylate (B-260)

tert-butyl 3-(1-(2,6-dioxopiperidin-3-yl)-3-methyl-2-oxo-2,3-dihydro-1H-benzo[d]imidazol-5-yl)azetidine-1-carboxylate (B-260, 0.8 g, 1.64 mmol, 77% yield) was synthesized from tert-butyl 3-(3-methyl-2-oxo-2,3-dihydro-1H-benzo[d]imidazol-5-yl)azetidine-1-carboxylate (B-259) and 3-bromopiperidine-2,6-dione (B-139) in a similar fashion to Compound B-193, except using 1.2 eq. lithium bis(trimethylsilyl)amide (1.0 M in THF) and 1.5 eq. B-139. The material was purified by silica gel column chromatography (40% Ethyl acetate in petroleum ether). MS (ES+): m/z 415.1 [M+H]⁺.

### Step 6: 3-[5-(azetidin-3-yl)-3-methyl-2-oxo-benzimidazol-1-yl]piperidine-2,6-dione (B-261)

3-[5-(azetidin-3-yl)-3-methyl-2-oxo-benzimidazol-1-yl]piperidine-2,6-dione (B-261, 165 mg, 370.71 μmol, 24% yield, TFA salt) was synthesized from tert-butyl 3-(1-(2,6-dioxopiperidin-3-yl)-3-methyl-2-oxo-2,3-dihydro-1H-benzo[d]imidazol-5-yl)azetidine-1-carboxylate (B-260) in a similar fashion to Compound A-12, except using 2 eq. 4 M HCl in dioxane. The material was purified by reverse phase prep HPLC (Column: X-BRIDGE C18 (19×150 mm) 5 μm; Mobile phase A: 0.1% TFA in water and Mobile phase B: MeCN). MS (ES+): m/z 315.0 [M+H]⁺.

### 2-[1-[1-(2,6-dioxo-3-piperidyl)-3-methyl-2-oxo-benzimidazol-5-yl]azetidin-3-yl]oxyacetic acid (B-268)

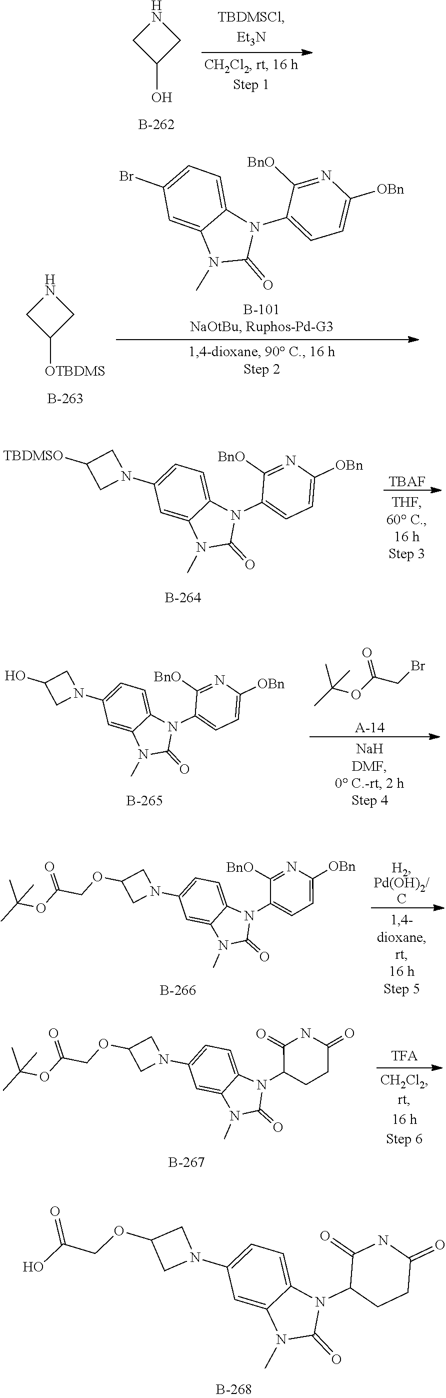

### Step 1: 3-((tert-butyldimethylsilyl)oxy)azetidine (B-263)

3-((tert-butyldimethylsilyl)oxy)azetidine (B-263, 3 g, 12.33 mmol, 68% yield) was synthesized from azetidin-3-ol (B-262) in a similar fashion to Compound B-248, except using 1.2 eq. tert-butyldimethylsilyl chloride. LCMS (ES+): m/z 188.2 [M+H]⁺.

### Step 2: 5-[3-[tert-butyl(dimethyl)silyl]oxyazetidin-1-yl]-1-(2,6-dibenzyloxy-3-pyridyl)-3-methyl-benzimidazol-2-one (B-264)

5-[3-[tert-butyl(dimethyl)silyl]oxyazetidin-1-yl]-1-(2,6-dibenzyloxy-3-pyridyl)-3-methyl-benzimidazol-2-one (B-264, 850 mg, 1.32 mmol, 68% yield) was synthesized from 5-bromo-1-(2,6-dibenzyloxy-3-pyridyl)-3-methyl-benzimidazol-2-one (B-101) and 3-((tert-butyldimethylsilyl)oxy)azetidine (B-263) in a similar fashion to Compound B-244, except using 2 eq. B-263 and 0.15 eq. RuPhos-Pd-G3. LCMS (ES+): m/z 623.3 [M+H]⁺.

### Step 3: 1-(2,6-dibenzyloxy-3-pyridyl)-5-(3-hydroxyazetidin-1-yl)-3-methyl-benzimidazol-2-one (B-265)

1-(2,6-dibenzyloxy-3-pyridyl)-5-(3-hydroxyazetidin-1-yl)-3-methyl-benzimidazol-2-one (B-265, 250 mg, 476.83 μmol, 74% yield) was synthesized from 5-[3-[tert-butyl(dimethyl)silyl]oxyazetidin-1-yl]-1-(2,6-dibenzyloxy-3-pyridyl)-3-methyl-benzimidazol-2-one (B-264) in a similar fashion to Compound B-250, except using 5 eq. tetrabutylammonium fluoride (1 M in THF). LCMS (ES+): m/z 509.1 [M+H]⁺.

### Step 4: tert-butyl 2-[1-[1-(2,6-dibenzyloxy-3-pyridyl)-3-methyl-2-oxo-benzimidazol-5-yl]azetidin-3-yl]oxyacetate (B-266)

tert-butyl 2-[1-[1-(2,6-dibenzyloxy-3-pyridyl)-3-methyl-2-oxo-benzimidazol-5-yl]azetidin-3-yl]oxyacetate (B-266, 200 mg, 276.21 μmol, 56% yield) was synthesized from 1-(2,6-dibenzyloxy-3-pyridyl)-5-(3-hydroxyazetidin-1-yl)-3-methyl-benzimidazol-2-one (B-265) and tert-butyl 2-bromoacetate (A-14) in a similar fashion to Compound B-3, except using 1.5 eq. NaH and 2 eq. B-265. The reaction was quenched with ammonium chloride solution. LCMS (ES+): m/z 623.2 [M+H]⁺.

### Step 5: tert-butyl 2-[1-[1-(2,6-dioxo-3-piperidyl)-3-methyl-2-oxo-benzimidazol-5-yl]azetidin-3-yl]oxyacetate (B-267)

tert-butyl 2-[1-[1-(2,6-dioxo-3-piperidyl)-3-methyl-2-oxo-benzimidazol-5-yl]azetidin-3-yl]oxyacetate (B-267, 70 mg, 121.27 μmol, 44% yield) was synthesized from tert-buty 12-[1-[1-(2,6-dibenzyloxy-3-pyridyl)-3-methyl-2-oxo-benzimidazol-5-yl]azetidin-3-yl]oxyacetate (B-266) in a similar fashion to Compound B-103, except using 0.6 eq. palladium hydroxide on carbon (20% by weight, 50% water). The Celite pad was washed with 1,4-dioxane and the material was triturated with diethyl ether. LCMS (ES+): m/z 445.1 [M+H]⁺.

### Step 6: 2-[1-[1-(2,6-dioxo-3-piperidyl)-3-methyl-2-oxo-benzimidazol-5-yl]azetidin-3-yl]oxyacetic acid (B-268)

2-[1-[1-(2,6-dioxo-3-piperidyl)-3-methyl-2-oxo-benzimidazol-5-yl]azetidin-3-yl]oxyacetic acid (B-268, 60 mg, 59.71 μmol, 53% yield, TFA salt) was synthesized from tert-butyl 2-[1-[1-(2,6-dioxo-3-piperidyl)-3-methyl-2-oxo-benzimidazol-5-yl]azetidin-3-yl]oxyacetate (B-267) in a similar fashion to Compound A-26. LCMS (ES+): m/z 389.0 [M+H]⁺.

### 1-[1-(2,6-dioxo-3-piperidyl)-3-methyl-2-oxo-benzimidazol-5-yl]piperidine-4-carboxylic acid (B-270)

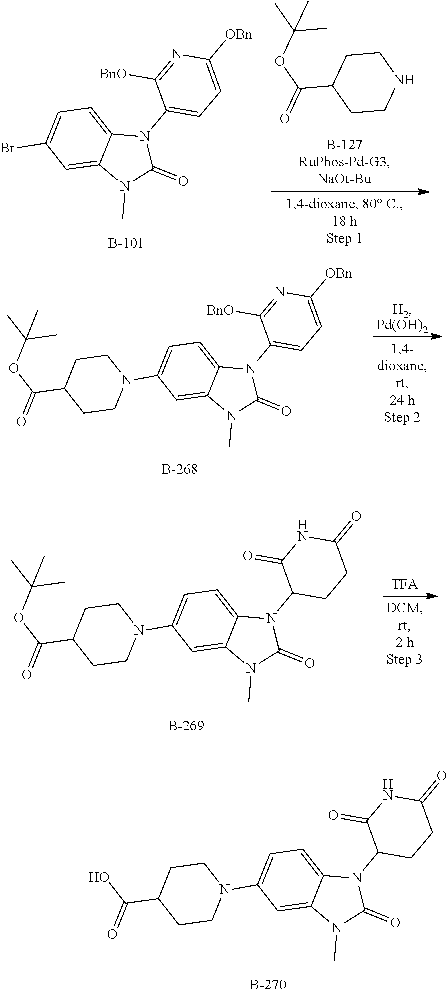

### Step 1: tert-butyl 1-[1-(2,6-dibenzyloxy-3-pyridyl)-3-methyl-2-oxo-benzimidazol-5-yl]piperidine-4-carboxylate (B-268)

tert-butyl 1-[1-(2,6-dibenzyloxy-3-pyridyl)-3-methyl-2-oxo-benzimidazol-5-yl]piperidine-4-carboxy late (B-268, 250 mg, 390.26 μmol, 41% yield) was synthesized from 5-bromo-1-(2,6-dibenzyloxy-3-pyridyl)-3-methyl-benzimidazol-2-one (B-101) and tert-butyl piperidine-4-carboxylate (B-127) in a similar fashion to Compound B-244, except using 1.2 eq. B-127 and 0.1 eq. RuPhos-Pd-G3. Upon completion, the mixture was filtered through Celite and washed with dioxane. The solvent was removed, and the residue purified by flash silica gel column chromatography (45% Ethyl acetate in petroleum ether). LCMS (ES+): m/z 621.3 [M+H]⁺.

### Step 2: ter-butyl 1-[1-(2,6-dioxo-3-piperidyl)-3-methyl-2-oxo-benzimidazol-5-yl]piperidine-4-carboxylate (B-269)

tert-butyl 1-[1-(2,6-dioxo-3-piperidyl)-3-methyl-2-oxo-benzimidazol-5-yl]piperidine-4-carboxylate (B-269, 140 mg, 280.00 μmol, 72% yield) was synthesized from tert-butyl 1-[1-(2,6-dibenzyloxy-3-pyridyl)-3-methyl-2-oxo-benzimidazol-5-yl]piperidine-4-carboxylate (B-268) in a similar fashion to Compound B-103, except using 0.2 eq. palladium hydroxide on carbon (20% by weight). LCMS (ES+): m/z 443.9 [M+H]⁺.

### Step 3: 1-[1-(2,6-dioxo-3-piperidyl)-3-methyl-2-oxo-benzimidazol-5-yl]piperidine-4-carboxylic acid (B-270)

1-[1-(2,6-dioxo-3-piperidyl)-3-methyl-2-oxo-benzimidazol-5-yl]piperidine-4-carboxylic acid (B-270, 97 mg, 167.86 μmol, 53% yield, TFA salt) was synthesized from tert-butyl 1-[1-(2,6-dioxo-3-piperidyl)-3-methyl-2-oxo-benzimidazol-5-yl]piperidine-4-carboxylate (B-269) in a similar fashion to Compound A-26, except using 1 eq. TFA. LCMS (ES+): m/z 387.1 [M+H]⁺.

### 3-[5-(2,7-diazaspiro[3.5]nonan-2-yl)-3-methyl-2-oxo-benzimidazol-1-yl]piperidine-2,6-dione (B-274)

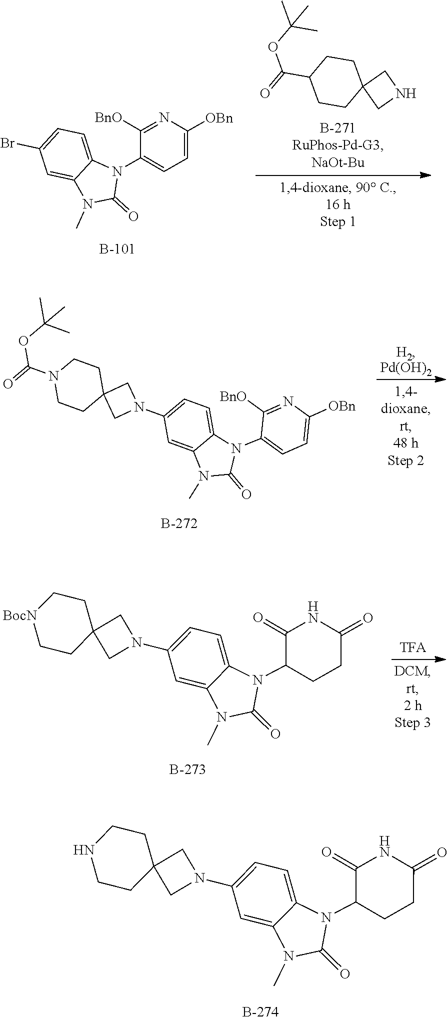

### Step 1: tert-butyl 2-[1-(2,6-dibenzyloxy-3-pyridyl)-3-methyl-2-oxo-benzimidazol-5-yl]-2,7-diazaspiro[3.5]nonane-7-carboxylate (B-272)

tert-butyl 2-[1-(2,6-dibenzyloxy-3-pyridyl)-3-methyl-2-oxo-benzimidazol-5-yl]-2,7-diazaspiro[3.5]nonane-7-carboxylate (B-272, 280 mg, 407.44 μmol, 42% yield) was synthesized from 5-bromo-1-(2,6-dibenzyloxy-3-pyridyl)-3-methyl-benzimidazol-2-one (B-101) and tert-butyl 2,7-diazaspiro[3.5]nonane-7-carboxylate (B-271) in a similar fashion to Compound B-244, except using 1 eq. B-271, 1 eq. sodium tert-butoxide and 1 eq. RuPhos-Pd-G3. Upon completion, the mixture was filtered through Celite. The filtrate was concentrated under reduced pressure and the residue was purified via silica gel (230-400 mesh) column chromatography (45% Ethyl acetate in petroleum ether). LCMS (ES+): m/z 662.2 [M+H]⁺.

### Step 2: tert-butyl 2-[1-(2,6-dioxo-3-piperidyl)-3-methyl-2-oxo-benzimidazol-5-yl]-2,7-diazaspiro[3,5]nonane-7-carboxylate (B-273)

tert-butyl 2-[1-(2,6-dioxo-3-piperidyl)-3-methyl-2-oxo-benzimidazol-5-yl]-2,7-diazaspiro[3.5]nonane-7-carboxylate (B-273, 180 mg, 263.17 μmol, 65% yield) was synthesized from tert-butyl 2-[1-(2,6-dibenzyloxy-3-pyridyl)-3-methyl-2-oxo-benzimidazol-5-yl]-2,7-diazaspiro[3.5]nonane-7-carboxylate (B-272) in a similar fashion to Compound B-103, except using 0.2 eq. palladium hydroxide on carbon (20% by weight). The Celite pad was washed with 1,4-dioxane and the material was purified by reverse phase column chromatography [Purification method: Biotage C18-size 30 g, (150×19 mm) 5 μm; 0.1% TFA in MQ water/MeCN]. LCMS (ES+): m/z 484.9 [M+H]⁺.

### Step 3: 3-[5-(2,7-diazaspiro[3.5]nonan-2-yl)-3-methyl-2-oxo-benzimidazol-1-yl]piperidine-2,6-dione (B-274)

3-[5-(2,7-diazaspiro[3.5]nonan-2-yl)-3-methyl-2-oxo-benzimidazol-1-yl]piperidine-2,6-dione (B-274, 120 mg, 188.15 μmol, 51% yield, TFA salt) was synthesized from tert-butyl 2-[1-(2,6-dioxo-3-piperidyl)-3-methyl-2-oxo-benzimidazol-5-yl]-2,7-diazaspiro[3.5]nonane-7-carboxylate (B-273) in a similar fashion to Compound A-62, except using 1 eq. TFA. The material was triturated with diethyl ether. LCMS (ES+): m/z 383.9 [M+H]⁺.

### N-[5-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-S-yl]piperidine-1-carboxamide (B-275)

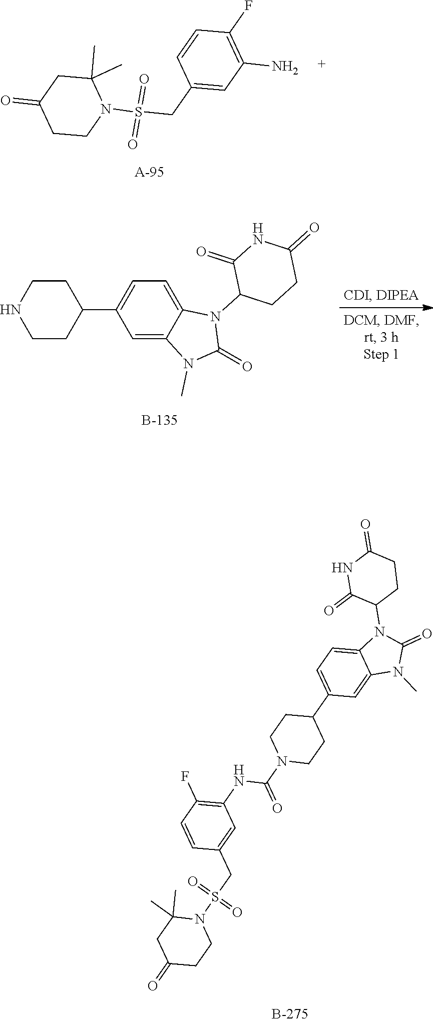

### Step 1: N-[5-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-275)

N-[5-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-275, 150 mg, 134.17 μmol, 42% yield) was synthesized from 1-[(3-amino-4-fluoro-phenyl)methylsulfonyl]-2,2-dimethyl-piperidin-4-one (A-95) and 3-[3-methyl-2-oxo-5-(4-piperidyl)benzimidazol-1-yl]piperidine-2,6-dione (B-135) in a similar fashion to Compound B-242, except using 1.1 eq. B-135 and 10 eq. DIPEA. Upon completion, the solvent was removed and the residue suspended in water. The precipitate was filtered and dried under vacuum. The material was used in the next step without further purification. LCMS (ES+): m/z 683.2 [M+H]⁺.

### N-[3-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-277)

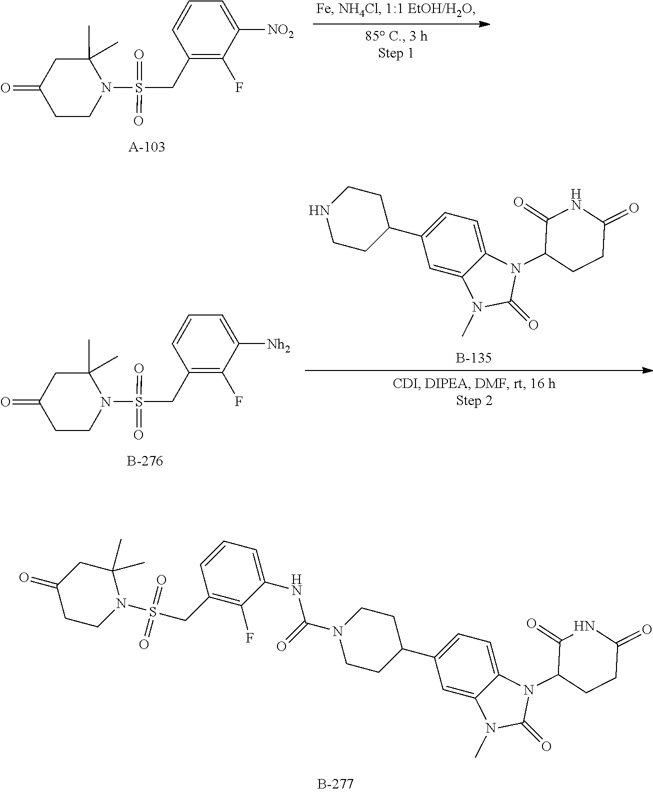

### Step 1: 1-[(3-amino-2-fluoro-phenyl)methylsulfonyl]-2,2-dimethyl-piperidin-4-one (B-276)

1-[(3-amino-2-fluoro-phenyl)methylsulfonyl]-2,2-dimethyl-piperidin-4-one (B-276, 120 mg, 288.65 μmol, 36% yield) was synthesized from 1-[(2-fluoro-3-nitro-phenyl)methylsulfonyl]-2,2-dimethyl-piperidin-4-one (B-103) in a similar fashion to Compound B-99, except using 1 eq. iron and 1 eq. ammonium chloride. Following filtration, the filtrate was diluted with Ethyl acetate and washed with water (2×). The combined organic layer was dried over anhydrous sodium sulfate, filtered and concentrated under reduced pressure. The residue was purified by flash silica gel (60-120 mesh) column chromatography (80-95% ethyl acetate in petroleum ether). LCMS (ES+): m/z 315 [M+H]⁺.

### Step 2: N-[3-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-277)

N-[3-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-277, 25 mg, 30.41 μmol, 8% yield) was synthesized from 3-[3-methyl-2-oxo-5-(4-piperidyl)benzimidazol-1-yl]piperidine-2,6-dione (B-135) and 1-[(3-amino-2-fluoro-phenyl)methylsulfonyl]-2,2-dimethyl-piperidin-4-one (B-276) in a similar fashion to Compound B-242, except using 1.3 eq. B-276, 2 eq. DIPEA and 1.3 eq. CDI. LCMS (ES+): m/z 683.3 [M+H]⁺.

### 3-[5-(azetidin-3-yloxy)-3-methyl-2-oxo-benzimidazol-1-yl]piperidine-2,6-dione (B-282)

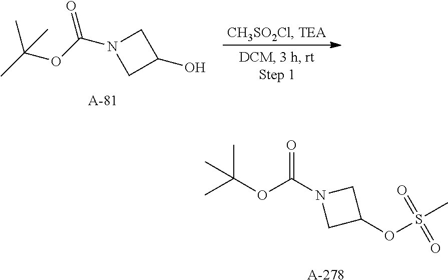

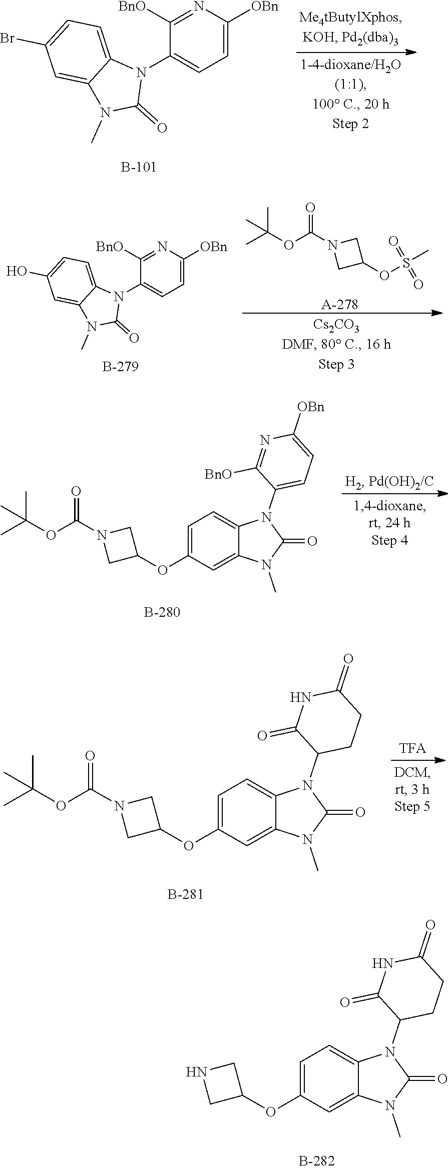

### Step 1: tert-butyl 3-((methylsulfonyl)oxy)azetidine-1-carboxylate (B-278)

tert-butyl 3-((methylsulfonyl)oxy)azetidine-1-carboxylate (B-278, 400 mg, 1.29 mmol, 74% yield) was synthesized from tert-butyl 3-hydroxyazetidine-1-carboxylate (A-81) in a similar fashion to Compound B-146, except using 2 eq. triethylamine and 1.1 eq. methanesulfonyl chloride. The reaction mixture was quenched with ice water and extracted with ethyl acetate (2×). The combined organic layer was washed with saturated sodium bicarbonate, water and brine, dried over sodium sulfate, filtered and solvent removed.

### Step 2:1-(2,6-dibenzyloxy-3-pyridyl)-5-hydroxy-3-methyl-benzimidazol-2-one (B-279)

Into a 50 mL pressure tube containing a well-stirred solution of 5-bromo-1-(2,6-dibenzyloxy-3-pyridyl)-3-methyl-benzimidazol-2-one (B-101, 500 mg, 968.27 μmol) in water (2 mL) and 1,4-dioxane (8 mL) was added KOH (135.81 mg, 2.42 mmol) and the reaction mixture was purged by bubbling nitrogen gas through for 15 min. Then tris(dibenzylideneacetone) dipalladium (0) (17.73 mg, 19.37 μmol) and tetramethyl di-(BuXPhos (23.27 mg, 48.41 μmol) were added. The vial was sealed and the mixture was stirred at 100° C. for 20 h. The reaction mixture was filtered through Celite and washed thoroughly with 1,4-dioxane (100 mL) and water (50 mL). The filtrate was concentrated under reduced pressure to remove dioxane and the aqueous layer was acidified using 1.5 N HCl (20 mL) and extracted with 10% methanol in DCM (2×150 mL). The combined organic layer was washed with water and brine, dried over sodium sulfate, filtered and solvent removed. The residue was purified by flash silica gel column chromatography (40-60% Ethyl acetate in pet-ether) to afford 1-(2,6-dibenzyloxy-3-pyridyl)-5-hydroxy-3-methyl-benzimidazol-2-one (B-279, 125 mg, 252.87 μmol, 26% yield) as an off-white solid. LCMS (ES+): m/z 454.1 [M+H]⁺.

### Step 3: tert-butyl 3-[1-(2,6-dibenzyloxy-3-pyridyl)-3-methyl-2-oxo-benzimidazol-5-yl]oxyazetidine-1-carboxylate (B-280)

tert-butyl 3-[1-(2,6-dibenzyloxy-3-pyridyl)-3-methyl-2-oxo-benzimidazol-5-yl]oxyazetidine-1-carboxylate (B-280, 200 mg, 239.86 μmol, 87% yield) was synthesized from 1-(2,6-dibenzyloxy-3-pyridyl)-5-hydroxy-3-methyl-benzimidazol-2-one (B-279) and tert-butyl 3-((methylsulfonyl)oxy)azetidine-1-carboxylate (B-278) in a similar fashion to Compound B-148, except using 2 eq. B-278. The reaction was quenched with ice water and extracted with DCM (2×). The combined organic layers were washed with water and brine, dried over anhydrous sodium sulfate, filtered, and solvent removed. The material was purified by flash silica gel column chromatography (30-40% of Ethyl acetate in petroleum ether). LCMS (ES+): m/z 609.3 [M+H]⁺.

### Step 4: tert-butyl 3-((1-(2,6-dioxopiperidin-3-yl)-3-methyl-2-oxo-2,3-dihydro-1H-benzo[d]imidazol-5-yl)oxy)azetidine-1-carboxylate (B-281)

tert-butyl 3-((1-(2,6-dioxopiperidin-3-yl)-3-methyl-2-oxo-2,3-dihydro-1H-benzo[d]imidazol-5-yl)oxy)azetidine-1-carboxylate (B-281, 110 mg, 160.99 μmol, 58% yield) was synthesized from tert-butyl 3-[1-(2,6-dibenzyloxy-3-pyridyl)-3-methyl-2-oxo-benzimidazol-5-yl]oxyazetidine-1-carboxylate (B-280) in a similar fashion to Compound B-103, except using 0.6 eq. palladium hydroxide on carbon (20% by weight). The Celite pad was washed with 1,4-dioxane. LCMS (ES+): m/z 331.2 [M−Boc+H]⁺.

### Step 5: 3-[5-(azetidin-3-yloxy)-3-methyl-2-oxo-benzimidazol-1-yl]piperidine-2,6-dione (B-282)

3-[5-(azetidin-3-yloxy)-3-methyl-2-oxo-benzimidazol-1-yl]piperidine-2,6-dione (B-282, 35 mg, 74.56 μmol, 25% yield, TFA salt) was synthesized from tert-butyl 3-((1-(2,6-dioxopiperidin-3-yl)-3-methyl-2-oxo-2,3-dihydro-1H-benzo[d]imidazol-5-yl)oxy)azetidine-1-carboxylate (B-281) in a similar fashion to Compound A-62, except using 5 eq. TFA. The material was purified by reverse phase column chromatography (50 g C18 column; 0.1% formic acid in water/MeCN). LCMS (ES+): m/z 331.1 [M+H]⁺.

### 3-[3-methyl-2-oxo-5-(4-piperidyloxy)benzimidazol-1-yl]piperidine-2,6-dione (B-285)

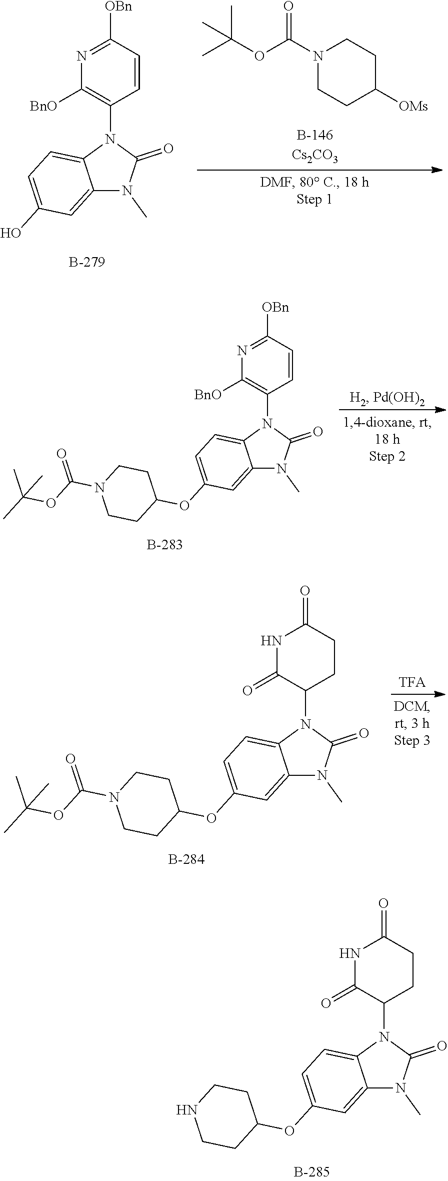

### Step 1: tert-butyl 4-[1-(2,6-dibenzyloxy-3-pyridyl)-3-methyl-2-oxo-benzimidazol-5-yl]oxypiperidine-1-carboxylate (B-283)

tert-butyl 4-[1-(2,6-dibenzyloxy-3-pyridyl)-3-methyl-2-oxo-benzimidazol-5-yl]oxypiperidine-1-carboxylate (B-283, 500 mg, 751.49 μmol, 68% yield) was synthesized from 1-(2,6-dibenzyloxy-3-pyridyl)-5-hydroxy-3-methyl-benzimidazol-2-one (B-279) and tert-butyl 4-methylsulfonyloxypiperidine-1-carboxylate (B-146) in a similar fashion to Compound B-148, except using 2 eq. B-146 and 1.5 eq. cesium carbonate. The reaction mixture was filtered and concentrated under reduced pressure. The residue was purified by flash silica gel column chromatography (40-50% ethyl acetate in pet-ether). LCMS (ES+): m/z 637.3 [M+H]⁺.

### Step 2: tert-butyl 4-[1-(2,6-dioxo-3-piperidyl)-3-methyl-2-oxo-benzimidazol-S-yl]oxypiperidine-1-carboxylate (B-284)

ter-butyl 4-[1-(2,6-dioxo-3-piperidyl)-3-methyl-2-oxo-benzimidazol-5-yl]oxypiperidine-1-carboxylate (B-284, 200 mg, 363.83 μmol, 77% yield) was synthesized from tert-butyl 4-[1-(2,6-dibenzyloxy-3-pyridyl)-3-methyl-2-oxo-benzimidazol-5-yl]oxypiperidine-1-carboxylate (B-283) in a similar fashion to Compound B-103, except using 0.2 eq. palladium hydroxide on carbon (20% by weight). The Celite pad was washed with 1,4-dioxane. LCMS (ES+): m/z 359.2 [M−Boc+H]⁺.

### Step 3: 3-[3-methyl-2-oxo-5-(4-piperidyloxy)benzimidazol-1-yl]piperidine-2,6-dione (B-285)

3-[3-methyl-2-oxo-5-(4-piperidyloxy)benzimidazol-1-yl]piperidine-2,6-dione (B-285, 150 mg, 282.69 μmol, 65% yield, TFA salt) was synthesized from tert-butyl 4-[1-(2,6-dioxo-3-piperidyl)-3-methyl-2-oxo-benzimidazol-5-yl]oxypiperidine-1-carboxylate (B-284) in a similar fashion to Compound A-62, except using 89 eq. TFA. The material was triturated with diethyl ether. LCMS (ES+): m/z 358.9 [M+H]⁺.

3-[2-oxo-3-(4-piperidyl)benzimidazol-1-yl]piperidine-2,6-dione (B-291)

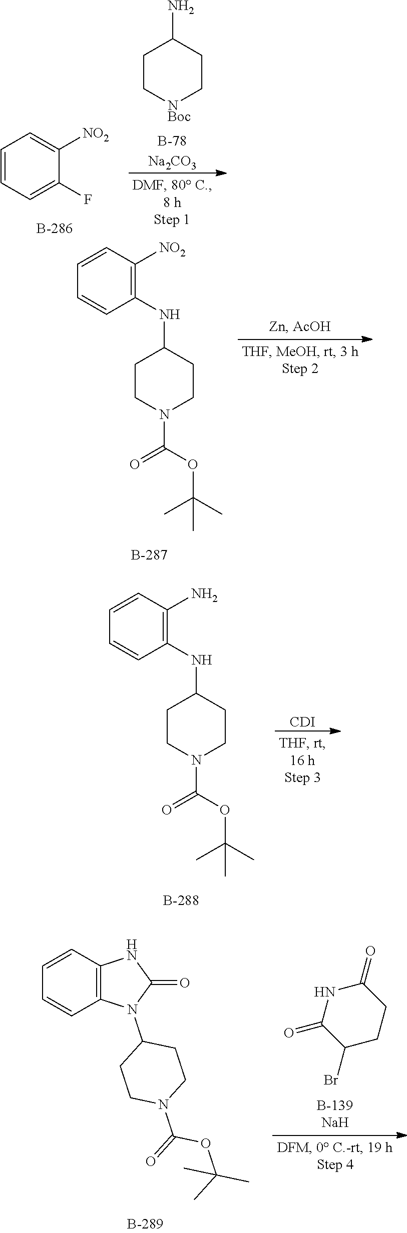

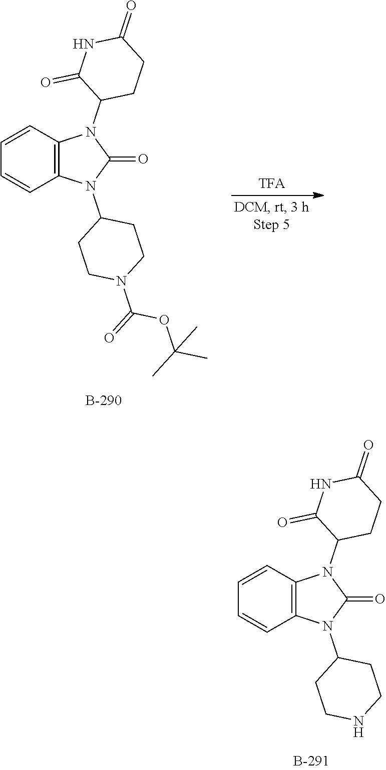 [SMILES: CC(C)(C)OC(=O)N1CCC(n2c(=O)n(C3CCC(=O)NC3=O)c3ccccc32)CC1 ; O=C1CCC(n2c(=O)n(C3CCNCC3)c3ccccc32)C(=O)N1]

### Step 1: tert-butyl 4-(2-nitroanilino)piperidine-1-carboxylate (B-287)

Into a 250 mL three neck round bottom flask containing a well-stirred solution of 1-fluoro-2-nitro-benzene (B-286, 5.0 g, 35.44 mmol, 3.73 mL) in DMF (50 mL) were added sodium carbonate (11.27 g, 106.31 mmol) and ter-butyl 4-aminopiperidine-1-carboxylate (B-78, 7.81 g, 38.98 mmol) at 0° C. Then the suspension was stirred at 80° C. for 8 h. The reaction mixture was filtered and concentrated under reduced pressure. The residue was diluted with water (50 mL) and extracted with ethyl acetate (3×50 mL). The combined organic layer was washed with water (40 mL) and brine (40 mL), dried over sodium sulfate, filtered and solvent removed under reduced pressure to obtain tert-butyl 4-(2-nitroanilino)piperidine-1-carboxylate (B-287, 10 g, 29.11 mmol, 82% yield) as yellow solid which was used in the next step without further purification. LCMS (ES+): m/z 222.0 [M−Boc+H]⁺.

### Step 2: tert-butyl 4-(2-aminoanilino)piperidine-1-carboxylate (B-288)

tert-butyl 4-(2-aminoanilino)piperidine-1-carboxylate (B-288, 9.0 g, 26.99 mmol, 87% yield) was synthesized from tert-butyl 4-(2-nitroanilino)piperidine-1-carboxylate (B-287) in a similar fashion to Compound A-10, except using 3 eq. zinc powder and 3 eq. Acetic acid. The material was used in the next step without purification. LCMS (ES+): m/z 292.0 [M+H]⁺.

### Step 3: tert-butyl 4-(2-oxo-3H-benzimidazol-1-yl)piperidine-1-carboxylate (B-289)

tert-butyl 4-(2-oxo-3H-benzimidazol-1-yl)piperidine-1-carboxylate (B-289, 4.5 g, 12.64 mmol, 41% yield) was synthesized from tert-butyl 4-(2-aminoanilino)piperidine-1-carboxylate (B-288) in a similar fashion to Compound B-208, except the reaction was started at 0° C. before allowing it to come to room temperature. Upon completion, the solvent was removed under reduced pressure and the residue taken up in DCM and washed with 1.5 N HCl (2×). The organic layer was dried over sodium sulfate, filtered and concentrated under reduced pressure. The residue was purified by flash silica gel column chromatography (40-60% Ethyl acetate in pet-ether). LCMS (ES+): m/z 218.2 [M−Boc+H]⁺.

### Step 4: tert-butyl 4-[3-(2,6-dioxo-3-piperidyl)-2-oxo-benzimidazol-1-yl]piperidine-1-carboxylate (B-290)

tert-butyl 4-[3-(2,6-dioxo-3-piperidyl)-2-oxo-benzimidazol-1-yl]piperidine-1-carboxylate (B-290, 500 mg, 1.10 mmol, 17% yield) was synthesized from tert-butyl 4-(2-oxo-3H-benzimidazol-1-yl)piperidine-1-carboxylate (B-289) and 3-bromopiperidine-2,6-dione (B-139) in a similar fashion to Compound B-3, except using 2 eq. NaH and 2 eq. B-139. The reaction was quenched with saturated ammonium chloride solution. LCMS (ES+): m/z 429.1 [M+H]⁺.

### Step 5: 3-[2-oxo-3-(4-piperidyl)benzimidazol-1-yl]piperidine-2,6-dione (B-291)

3-[2-oxo-3-(4-piperidyl)benzimidazol-1-yl]piperidine-2,6-dione (B-291, 70 mg, 146.67 μmol, 79% yield, TFA salt) was synthesized from tert-butyl 4-[3-(2,6-dioxo-3-piperidyl)-2-oxo-benzimidazol-1-yl]piperidine-1-carboxylate (B-290) in a similar fashion to Compound A-62, except using 3 eq. TFA. The material was triturated with diethyl ether. LCMS (ES+): m/z 329.0 [M+H]⁺.

### 2-[4-[3-(2,6-dioxo-3-piperidyl) indazol-1-yl]-1-piperidyl]acetic acid (B-299)

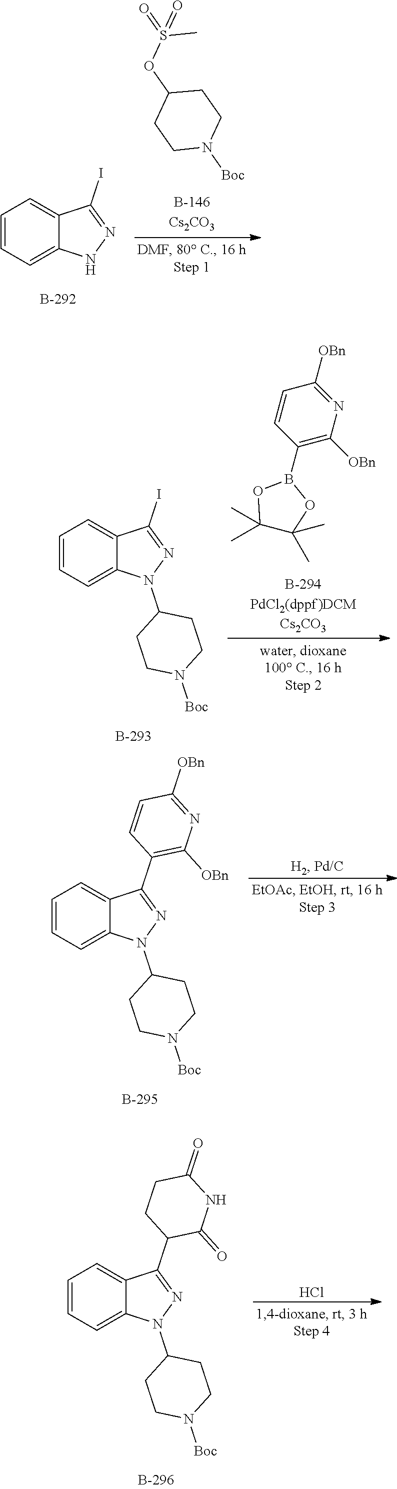

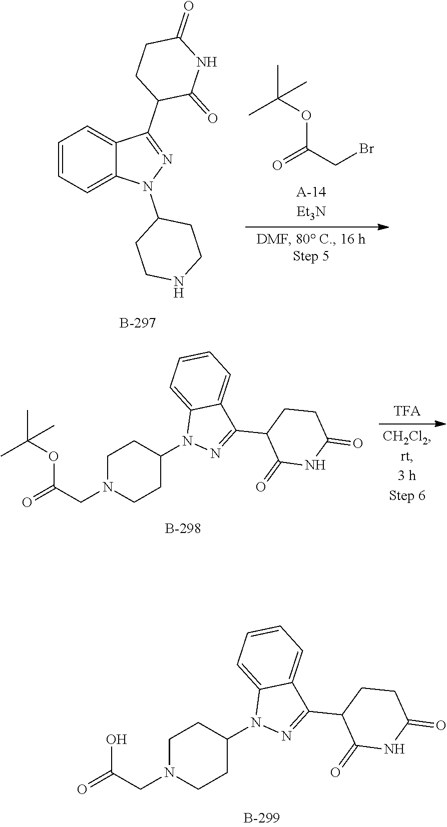

### Step 1: tert-butyl 4-(3-iodoindazol-1-yl)piperidine-1-carboxylate (B-293)

tert-butyl 4-(3-iodoindazol-1-yl)piperidine-1-carboxylate (B-293, 33.0 g, 77.23 mmol, 75.39% yield) was synthesized from 3-iodo-1H-indazole (B-292) and tert-butyl 4-methylsulfonyloxypiperidine-1-carboxylate (B-146) in a similar fashion to Compound B-148, except using 3 eq. cesium carbonate and 1.7 eq. B-146. LCMS (ES+): m/z 428.0 [M+H]⁺.

### Step 2: tert-butyl 4-[3-(2,6-dibenzyloxy-3-pyridyl) indazol-1-yl]piperidine-1-carboxylate (B-295)

To a stirred solution of tert-butyl 4-(3-iodoindazol-1-yl)piperidine-1-carboxylate (B-293, 28.0 g, 65.53 mmol) in water (112 mL) and 1,4-dioxane (448 mL) were added 2,6-dibenzyloxy-3-(4,4,5,5-tetramethyl-1,3,2-dioxaborolan-2-yl)pyridine (B-294, 41.02 g, 98.30 mmol) and Cs₂CO₃ (164.92 g, 196.59 mmol) and the reaction mixture was purged with nitrogen for 15 minutes. Then PdCl₂(dppf)DCM (5.35 g, 6.55 mmol) was added, and the reaction mixture was heated at 100° C. for 16 h. The reaction mixture was diluted with ethyl acetate, washed with water, dried over sodium sulfate, filtered and concentrated. The residue purified by column chromatography using (silica gel 100-200 mesh), eluting with 80-20% DCM/Hexane to afford tert-butyl 4-[3-(2,6-dibenzyloxy-3-pyridyl) indazol-1-yl]piperidine-1-carboxylate (B-295, 24.5 g, 41.48 mmol, 63% yield) as a gummy solid. LCMS (ES+): m/z 591.3 [M+H]⁺.

### Step 3: tert-butyl 4-[3-(2,6-dioxo-3-piperidyl) indazol-1-yl]piperidine-1-carboxylate (B-296)

A stirred solution of tert-butyl 4-[3-(2,6-dibenzyloxy-3-pyridyl) indazol-1-yl]piperidine-1-carboxylate (B-295, 28.0 g, 47.40 mmol) in Ethanol (150 mL) and Ethyl acetate (150 mL) was degassed for 15 minutes under argon atmosphere before addition of palladium (10% on carbon, wet) (7.57 g, 7.10 mmol). The reaction mixture was stirred under hydrogen atmosphere (40 psi) for 16 h. The reaction mixture was filtered through Celite, washing with ethanol. The filtrate was concentrated and the residue purified by column chromatography, eluting with 0-1% MeOH:DCM, to afford tert-butyl 4-[3-(2,6-dioxo-3-piperidyl) indazol-1-yl]piperidine-1-carboxy late (B-296, 14.5 g, 35.15 mmol, 74% yield) as a white solid. LCMS (ES+): m/z 413.2 [M+H]⁺.

### Step 4: 3-[1-(4-piperidyl) indazol-3-yl]piperidine-2,6-dione (B-297)

To a stirred solution of tert-butyl 4-[3-(2,6-dioxo-3-piperidyl) indazol-1-yl]piperidine-1-carboxylate (B-296, 14.0 g, 33.94 mmol) in 1,4-Dioxane (10 mL) was added 4 M HCl in 1,4-Dioxane (15 mL) at 0° C. The reaction mixture was stirred for 4 hours at room temperature. The solvent was evaporated, and the crude was washed with diethyl ether to afford 3-[1-(4-piperidyl) indazol-3-yl]piperidine-2,6-dione (B-297, 10.5 g, 33.61 mmol, 99% yield) as an off-white solid. The material was used in the next step without further purification. LCMS (ES+): m/z 313.4 [M+H]⁺.

### Step 5: tert-butyl 2-[4-[3-(2,6-dioxo-3-piperidyl) indazol-1-yl]-1-piperidyl]acetate (B-298)

tert-butyl 2-[4-[3-(2,6-dioxo-3-piperidyl) indazol-1-yl]-1-piperidyl]acetate (B-298, 50 mg, 104.34 μmol) was synthesized from of 3-[1-(4-piperidyl) indazol-3-yl]piperidine-2,6-dione (B-297), and tert-butyl bromoacetate (A-14) in a similar fashion to Compound B-82, except using 1.2 eq. A-14 and 3 eq. triethylamine. The reaction was heated at 80° C. for 16 h. LCMS (ES+): m/z 427.2 [M+H]⁺.

### Step 6: 2-[4-[3-(2,6-dioxo-3-piperidyl)) indazol-1-yl]-1-piperidyl]acetic acid (B-299)

2-[4-[3-(2,6-dioxo-3-piperidyl) indazol-1-yl]-1-piperidyl]acetic acid (B-299, 90 mg, 194.38 μmol, TFA salt) was synthesized from tert-butyl 2-[4-[3-(2,6-dioxo-3-piperidyl) indazol-1-yl]-1-piperidyl]acetate (B-298) in a similar fashion to Compound A-26, except using 5 eq. TFA. UPLC (ES+): m/z 371.8 [M+H]⁺.

### 3-[5-(3-hydroxyazetidin-1-yl)-3-methyl-2-oxo-benzimidazol-1-yl]piperidine-2,6-dione (B-300)

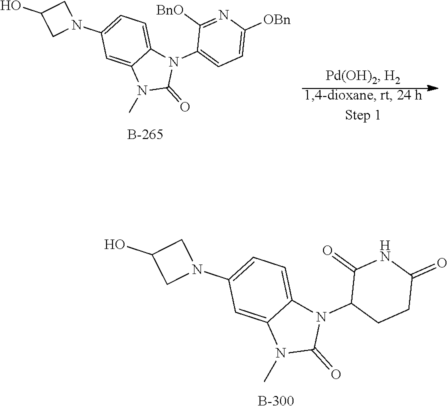 [SMILES: Cn1c(=O)n(-c2ccc(OCc3ccccc3)nc2OCc2ccccc2)c2ccc(N3CC(O)C3)cc21 ; Cn1c(=O)n(C2CCC(=O)NC2=O)c2ccc(N3CC(O)C3)cc21]

### Step 1: 3-[5-(3-hydroxyazetidin-1-yl)-3-methyl-2-oxo-benzimidazol-1-yl]piperidine-2,6-dione (B-300)

3-[5-(3-hydroxyazetidin-1-yl)-3-methyl-2-oxo-benzimidazol-1-yl]piperidine-2,6-dione (B-300, 20 mg, 42.51 μmol, 5% yield, formic acid salt) was synthesized from 1-(2,6-dibenzyloxy-3-pyridyl)-5-(3-hydroxyazetidin-1-yl)-3-methyl-benzimidazol-2-one (B-265) in a similar fashion to Compound B-103, except using 0.1 eq. palladium hydroxide (10% on carbon). The material was purified by prep HPLC (Column: SUNFIRE C18 (150×19 mm), 5 μm, mobile phase: 0.1% forming acid water:MeCN). LCMS (ES+): m/z 330.9 [M+H]⁺.

### 3-[2′-oxo-5′-(4-piperidyl)spiro[cyclopropane-1,3′-indoline]-1′-yl]piperidine-2,6-dione (B-305)

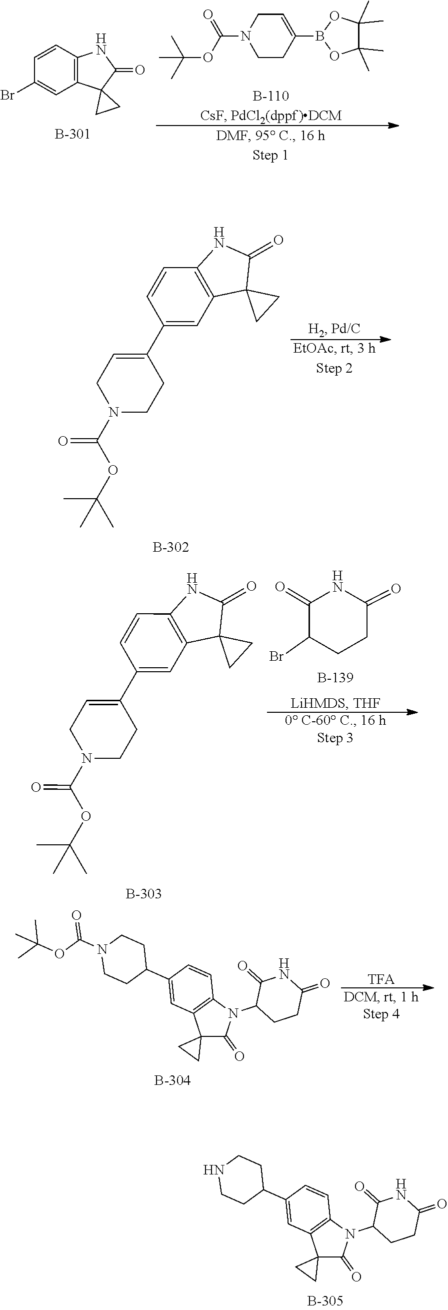

### Step 1: tert-butyl 4-(2′-oxospiro[cyclopropane-1,3′-indoline]-6′-yl)-3,6-dihydro-2H-pyridine-1-carboxylate (B-302)

tert-butyl 4-(2′-oxospiro[cyclopropane-1,3′-indoline]-6′-yl)-3,6-dihydro-2H-pyridine-1-carboxylate (B-302, 420 mg, 34.72% yield) was synthesized from 5′-bromospiro[cyclopropane-1,3′-indoline]-2′-one (B-301) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 1.5 eq. B-110, 0.1 eq. [1,1′-Bis(diphenylphosphino)ferrocene]dichloropalladium(II) complex with dichloromethane, 1.7 eq. CsF, The reaction was filtered through Celite prior to the work-up. LCMS (ES+): m/z 241.0 [M−Boc+H]⁺.

### Step 2: tert-butyl 4-(2′-oxospiro[cyclopropane-1,3′-indoline]-5′-yl)piperidine-1-carboxylate (B-303)

tert-butyl 4-(2′-oxospiro[cyclopropane-1,3′-indoline]-5′-yl)piperidine-1-carboxylate (B-303, 178 mg, 46.36% yield) was synthesized from tert-butyl 4-(2′-oxospiro[cyclopropane-1,3′-indoline]-6′-yl)-3,6-dihydro-2H-pyridine-1-carboxylate (B-302) in a similar fashion to Compound B-6. LCMS (ES+): m/z 243 [M−Boc+H]⁺.

### Step 3: tert-butyl 4-[1′-(2,6-dioxo-3-piperidyl)-2′-oxo-spiro[cyclopropane-1,3′-indoline]-5′-yl]piperidine-1-carboxylate (B-304)

tert-butyl 4-[1′-(2,6-dioxo-3-piperidyl)-2′-oxo-spiro[cyclopropane-1,3′-indoline]-5′-yl]piperidine-1-carboxy late (B-304, 34 mg, 17.86% yield) was synthesized from tert-butyl 4-(2′-oxospiro[cyclopropane-1,3′-indoline]-5′-yl)piperidine-1-carboxylate (B-303) and 3-bromopiperidine-2,6-dione (B-139) in a similar fashion to Compound B-193, except using 1.2 eq. lithium bis(trimethylsilyl)amide (1.0 M in THF) and 1.5 eq. B-139. The material was purified by flash silica gel (230-400 mesh) column chromatography (50% ethyl acetate in petroleum ether). LCMS (ES+): m/z 353.9 [M−Boc+H]⁺.

### Step 4: 3-[2′-oxo-5′-(4-piperidyl)spiro[cyclopropane-1,3′-indoline]-1′-yl]piperidine-2,6-dione (B-305)

3-[2′-oxo-5′-(4-piperidyl)spiro[cyclopropane-1,3′-indoline]-1′-yl]piperidine-2,6-dione (B-305, 240 mg, 475.36 μmol, 95.39% yield, 70% purity, TFA salt) was synthesized from tert-butyl 4-[1′-(2,6-dioxo-3-piperidyl)-2′-oxo-spiro[cyclopropane-1,3′-indoline]-5′-yl]piperidine-1-carboxylate (B-304) in a similar fashion to Compound A-62, except using 44 eq. TFA. LCMS (ES+): m/z 354.2 [M+H]⁺.

### 3-[3-methyl-2-oxo-4-(4-piperidyloxy)benzimidazol-1-yl]piperidine-2,6-dione (B-309)

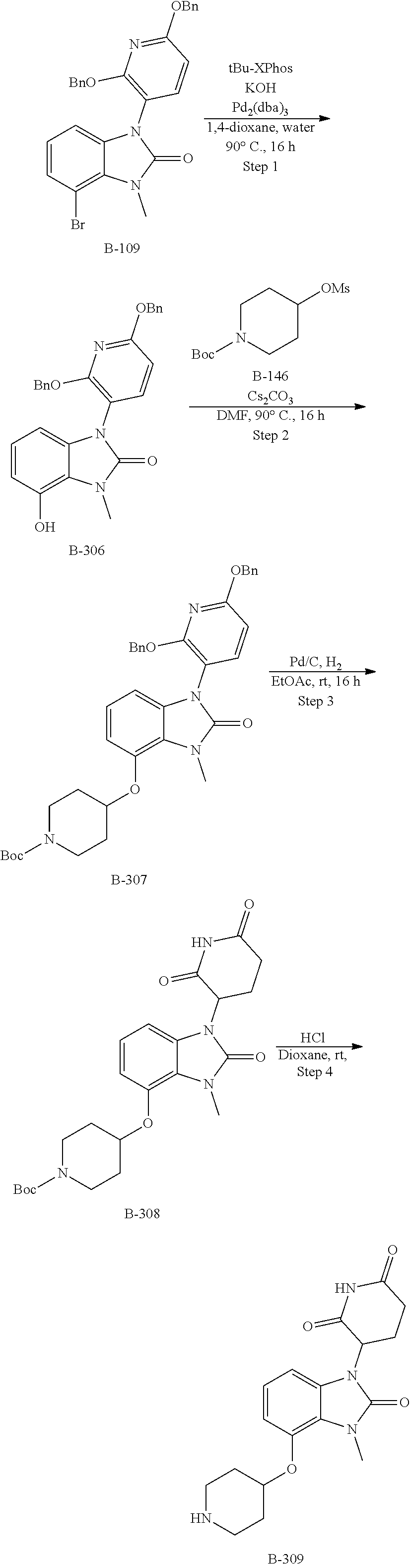 [SMILES: Cn1c(=O)n(-c2ccc(OCc3ccccc3)nc2OCc2ccccc2)c2cccc(Br)c21 ; Cn1c(=O)n(-c2ccc(OCc3ccccc3)nc2OCc2ccccc2)c2cccc(O)c21 ; CC(C)(C)OC(=O)N1CCC(OS(C)(=O)=O)CC1 ; Cn1c(=O)n(-c2ccc(OCc3ccccc3)nc2OCc2ccccc2)c2cccc(OC3CCN(C(=O)OC(C)(C)C)CC3)c21 ; Cn1c(=O)n(C2CCC(=O)NC2=O)c2cccc(OC3CCN(C(=O)OC(C)(C)C)CC3)c21 ; Cn1c(=O)n(C2CCC(=O)NC2=O)c2cccc(OC3CCNCC3)c21]

### Step 1: 1-(2,6-dibenzyloxy-3-pyridyl)-4-hydroxy-3-methyl-benzimidazol-2-one (B-306)

To a stirred solution of 4-bromo-1-(2,6-dibenzyloxy-3-pyridyl)-3-methyl-benzimidazol-2-one (B-109, 1 g, 1.94 mmol) in 1,4-dioxane (8 mL) was added KOH (239.03 mg, 4.26 mmol, 117.17 μL) dissolved in Water (8 mL) and purged with argon for 15 minutes. Di-tert-butyl-[2-(2,4,6-triisopropylphenyl)phenyl]phosphane (65.79 mg, 154.92 μmol) and tris(dibenzylideneacetone) dipalladium (0) (177.33 mg, 193.65 μmol) were added and purged with Ar for 10 minutes. The reaction mixture was heated to 90° C. for 16 h. The reaction mixture was diluted with ethyl acetate, filtered through Celite, washing with ethyl acetate. The combined organic layer was washed with water and brine, dried over anhydrous sodium sulfate, filtered and concentrated under reduced pressure. The residue was purified by column chromatography (35% ethyl acetate-hexane) to afford 1-(2,6-dibenzyloxy-3-pyridyl)-4-hydroxy-3-methyl-benzimidazol-2-one (B-306, 800 mg, 1.75 mmol, 90% yield). LCMS (ES+): m/z 454.2 [M+H]⁺.

### Step 2: tert-butyl 4-[1-(2,6-dibenzyloxy-3-pyridyl)-3-methyl-2-oxo-benzimidazol-4-yl]oxypiperidine-1-carboxylate (B-307)

tert-butyl 4-[1-(2,6-dibenzyloxy-3-pyridyl)-3-methyl-2-oxo-benzimidazol-4-yl]oxypiperidine-1-carboxy late (B-307, 1.65 g, 2.44 mmol, 50% yield) was synthesized from 1-(2,6-dibenzyloxy-3-pyridyl)-4-hydroxy-3-methyl-benzimidazol-2-one (B-306) and tert-butyl 4-methylsulfonyloxypiperidine-1-carboxylate (B-146) in a similar fashion to Compound B-148, except using 3 eq. cesium carbonate. LCMS (ES+): m/z 637.5 [M+H]⁺.

### Step 3: tert-butyl 4-[1-(2,6-dioxo-3-piperidyl)-3-methyl-2-oxo-benzimidazol-4-yl]oxypiperidine-1-carboxylate (B-308)

tert-butyl 4-[1-(2,6-dioxo-3-piperidyl)-3-methyl-2-oxo-benzimidazol-4-yl]oxypiperidine-1-carboxylate (B-308, 1.42 g, 3.04 mmol, 55% yield) was synthesized from tert-butyl 4-[1-(2,6-dibenzyloxy-3-pyridyl)-3-methyl-2-oxo-benzimidazol-4-yl]oxypiperidine-1-carboxylate (B-307) in a similar fashion to Compound B-6, except the material was purified by silica gel column chromatography, eluting with 50% ethyl acetate in hexane. LCMS (ES+): m/z 403.2 [M−tBu+H]⁺.

### Step 4: 3-[3-methyl-2-oxo-4-(4-piperidyloxy)benzimidazol-1-yl]piperidine-2,6-dione (B-309)

3-[3-methyl-2-oxo-4-(4-piperidyloxy)benzimidazol-1-yl]piperidine-2,6-dione (B-309, 1.25 g, 3.13 mmol, 96% yield, HCl salt) was synthesized from tert-butyl 4-[1-(2,6-dioxo-3-piperidyl)-3-methyl-2-oxo-benzimidazol-4-yl]oxypiperidine-1-carboxylate (B-308) in a similar fashion to Compound A-12, except using 10 eq. 4 M HCl in dioxane. LCMS (ES+): m/z 359.3 [M+H]⁺.

### 2-[4-[3-(2,6-dioxo-3-piperidyl)-2-oxo-benzimidazol-1-yl]-1-piperidyl]acetic acid (B-311)

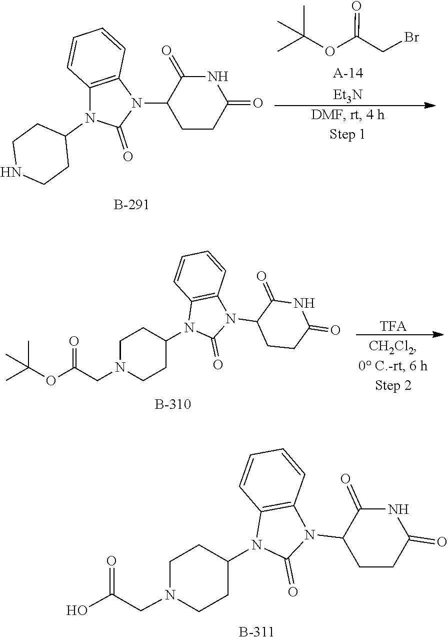

### Step 1: tert-butyl 2-[4-[3-(2,6-dioxo-3-piperidyl)-2-oxo-benzimidazol-1-yl]-1-piperidyl]acetate (B-310)

Into a 50 mL single neck round bottom flask containing a well-stirred solution of 3-[2-oxo-3-(4-piperidyl)benzimidazol-1-yl]piperidine-2,6-dione (B-291, 400 mg, 904.18 μmol, TFA salt) in DMF (2 mL) at 0° C. under inert atmosphere was added Et₃N (457.47 mg, 4.52 mmol, 630.13 μL) followed by tert-butyl 2-bromoacetate (A-14, 211.64 mg, 1.09 mmol, 159.13 μL) and the resulting mixture was stirred at room temperature for 4 h. The reaction mixture was quenched with ice water (100 mL) and extracted with Ethyl acetate (2×100 mL). The combined organic layer was washed with brine solution (2×50 mL), dried over sodium sulfate, filtered and concentrated under reduced pressure to afford tert-butyl 2-[4-[3-(2,6-dioxo-3-piperidyl)-2-oxo-benzimidazol-1-yl]-1-piperidyl]acetate (B-310, 360 mg, 792.80 μmol, 88% yield) as an off-white solid. LCMS (ES+): m/z 443.0 [M+H]⁺.

### Step 2: 2-[4-[3-(2,6-dioxo-3-piperidyl)-2-oxo-benzimidazol-1-yl]-1-piperidyl]acetic acid (B-311)

2-[4-[3-(2,6-dioxo-3-piperidyl)-2-oxo-benzimidazol-1-yl]-1-piperidyl]acetic acid (B-311, 180 mg, 291.78 μmol, 65% yield. TFA salt) was synthesized from tert-butyl 2-[4-[3-(2,6-dioxo-3-piperidyl)-2-oxo-benzimidazol-1-yl]-1-piperidyl]acetate (B-310) in a similar fashion to Compound A-26, except using 5 eq. TFA. LCMS (ES+): m/z 386.9 [M+H]⁺.

### (1s,4s)-4-(1-(2,6-dioxopiperidin-3-yl)-3-methyl-2-oxo-2,3-dihydro-1H-benzo[d]imidazol-5-yl)cyclohexane-1-carboxylic acid (B-315a, first eluted fraction) and (1r,4r)-4-(1-(2,6-dioxopiperidin-3-yl)-3-methyl-2-oxo-2,3-dihydro-1H-benzo[d]imidazol-5-yl)cyclohexane-1-carboxylic acid (B-315b, Second Eluted Fraction)

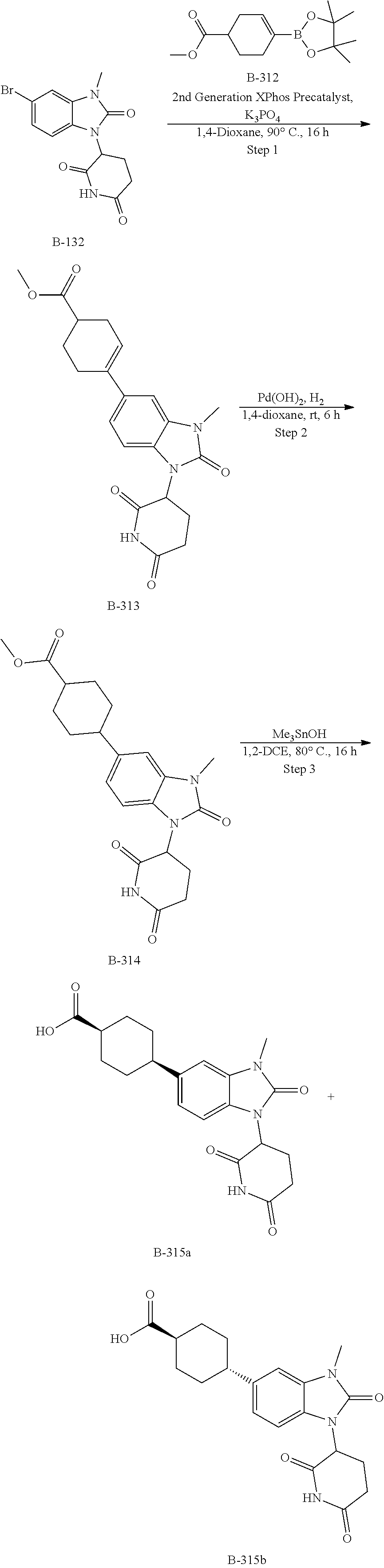

### Step 1: methyl 4-[1-(2,6-dioxo-3-piperidyl)-3-methyl-2-oxo-benzimidazol-5-yl]cyclohex-3-ene-1-carboxylate (B-313)

Into a 100 mL pressure tube containing a well-stirred solution of 3-(5-bromo-3-methyl-2-oxo-benzimidazol-1-yl)piperidine-2,6-dione (B-132, 1.25 g, 3.70 mmol) and methyl 4-(4,4,5,5-tetramethyl-1,3,2-dioxaborolan-2-yl)cyclohex-3-ene-1-carboxylate (B-312, 1.77 g, 6.65 mmol) in anhydrous 1,4-dioxane (15 mL) was added tripotassium phosphate (977.33 mg, 4.60 mmol). The suspension was purged by bubbling nitrogen through for 5 minutes. Then, 2nd Generation XPhos precatalyst (290.84 mg, 369.65 μmol) was added. The reaction mixture was heated at 90° C. for 16 h. The reaction mixture was diluted with ethyl acetate (10 mL) and filtered through Celite, washing with ethyl acetate (10 mL). The organic layer was washed with water (30 mL), dried over anhydrous sodium sulfate, filtered and concentrated under reduced pressure. The residue was triturated with diethyl ether, filtered and dried to afford methyl 4-[1-(2,6-dioxo-3-piperidyl)-3-methyl-2-oxo-benzimidazol-5-yl]cyclohex-3-ene-1-carboxylate (B-313, 580 mg, 919.42 μmol, 25% yield) as an off-white solid. The material was used in the next step without further purification. LCMS (ES+): m/z 398.2 [M+H]⁺.

### Step 2: methyl 4-[1-(2,6-dioxo-3-piperidyl)-3-methyl-2-oxo-benzimidazol-5-yl]cyclohexanecarboxylate (B-314)

methyl 4-[1-(2,6-dioxo-3-piperidyl)-3-methyl-2-oxo-benzimidazol-5-yl]cyclohexanecarboxylate (B-314, 550 mg, 1.13 mmol, 77.37% yield, 82% purity) was synthesized from methyl 4-[1-(2,6-dioxo-3-piperidyl)-3-methyl-2-oxo-benzimidazol-5-yl]cyclohex-3-ene-1-carboxylate (B-313) in a similar fashion to Compound B-103, except using 0.14 eq. palladium hydroxide (10% on carbon). LCMS (ES+): m/z 400.1 [M+H]⁺.

### Step 3: (1S,4S)-4-(1-(2,6-dioxopiperidin-3-yl)-3-methyl-2-oxo-2,3-dihydro-1H-benzo[d]imidazol-5-yl)cyclohexane-1-carboxylic acid (B-315a, First Eluted Fraction) and (1R,4R)-4-(1-(2,6-dioxopiperidin-3-yl)-3-methyl-2-oxo-2,3-dihydro-1H-benzo[d]imidazol-5-yl)cyclohexane-1-carboxylic acid (B-315b, Second Eluted Fraction)

Configurations are arbitrarily assigned.

Into a 50 mL single neck round bottom flask containing well-stirred solution of methyl 4-[1-(2,6-dioxo-3-piperidyl)-3-methyl-2-oxo-benzimidazol-5-yl]cyclohexanecarboxylate (B-314, 550 mg, 1.38 mmol) in 1,2-DCE (20 mL) was added trimethyltin hydroxide (2.49 g, 13.77 mmol). The resulting suspension was stirred at 80° C. for 16 h. The reaction mixture was diluted with DCM (10 mL) and filtered through Celite. The filtrate was concentrated under reduced pressure and the residue purified by reverse phase Prep-HPLC (Purification method: Column: XBridge C18 (19×150 mm) 5 μm; Mobile phase A: 0.1% TFA in water and mobile phase B: Acetonitrile) to afford (1S,4S)-4-(1-(2,6-dioxopiperidin-3-yl)-3-methyl-2-oxo-2,3-dihydro-1H-benzo[d]imidazol-5-yl)cyclohexane-1-carboxylic acid (B-315a, first eluted fraction, 100 mg, 181.54 μmol, 13% yield, TFA salt) as an off-white solid. LCMS (ES+): m/z 386.1 [M+H]⁺. (1R,4R)-4-(1-(2,6-dioxopiperidin-3-yl)-3-methyl-2-oxo-2,3-dihydro-1H-benzo[d]imidazol-5-yl)cyclohexane-1-carboxylic acid (B-315b, second eluted fraction, 100 mg, 181.85 μmol, 13% yield, TFA salt) as an off-white solid. LCMS (ES+): m/z 386.1 [M+H]⁺.

### 3-[5-(2,6-diazaspiro[3.3]heptan-2-yl)-3-methyl-2-oxo-benzimidazol-1-yl]piperidine-2,6-dione 319)

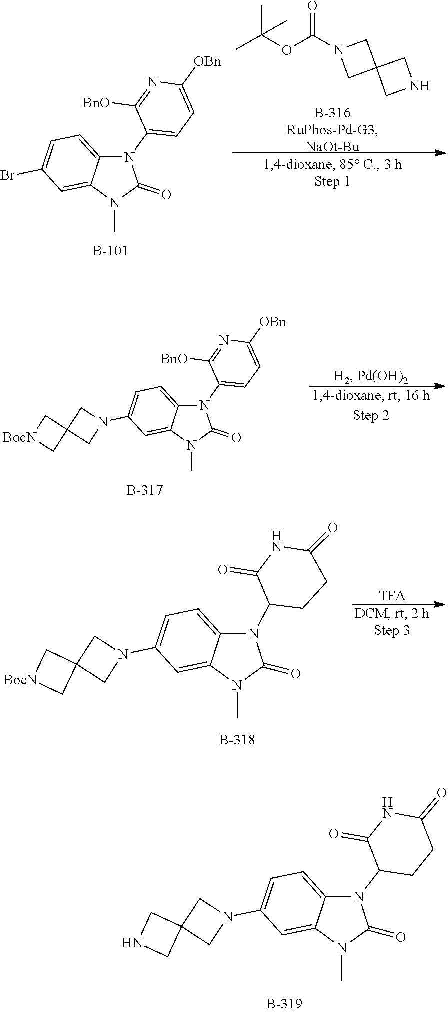

### Step 1: tert-butyl 6-[1-(2,6-dibenzyloxy-3-pyridyl)-3-methyl-2-oxo-benzimidazol-5-yl]-2,6-diazaspiro[3.3]heptane-2-carboxylate (B-317)

tert-butyl 6-[1-(2,6-dibenzyloxy-3-pyridyl)-3-methyl-2-oxo-benzimidazol-5-yl]-2,6-diazaspiro[3.3]heptane-2-carboxylate (B-317, 230 mg, 352.04 μmol, 63% yield) was synthesized from 5-bromo-1-(2,6-dibenzyloxy-3-pyridyl)-3-methyl-benzimidazol-2-one (B-101) and tert-butyl 2,6-diazaspiro[3.3]heptane-2-carboxylate (B-316) in a similar fashion to Compound B-244, except using 2 eq. B-316 and 0.1 eq. RuPhos-Pd-G3. Upon completion, the mixture was filtered through Celite, washing with Ethyl acetate. The organic layer was washed with water, dried over sodium sulfate, filtered and concentrated. The material was purified by flash silica gel column chromatography (40-50% Ethyl acetate in petroleum ether). LCMS (ES+): m/z 634.2 [M+H]⁺.

### Step 2: tert-butyl 6-[1-(2,6-dioxo-3-piperidyl)-3-methyl-2-oxo-benzimidazol-5-yl]-2,6-diazaspiro[3.3]heptane-2-carboxylate (B-318)

tert-butyl 6-[1-(2,6-dioxo-3-piperidyl)-3-methyl-2-oxo-benzimidazol-5-yl]-2,6-diazaspiro[3.3]heptane-2-carboxylate (B-318, 130 mg, 265.70 μmol, 73% yield) was synthesized from tert-butyl 6-[1-(2,6-dibenzyloxy-3-pyridyl)-3-methyl-2-oxo-benzimidazol-5-yl]-2,6-diazaspiro[3.3]heptane-2-carboxylate (B-317) in a similar fashion to Compound B-103, except the material was purified by flash silica gel column chromatography (70-100% Ethyl acetate in petroleum ether). LCMS (ES−): m/z 454.2 [M−H]⁻.

### Step 3: 3-[5-(2,6-diazaspiro[3.3]heptan-2-yl)-3-methyl-2-oxo-benzimidazol-1-yl]piperidine-2,6-dione (B-319)

3-[5-(2,6-diazaspiro[3.3]heptan-2-yl)-3-methyl-2-oxo-benzimidazol-1-yl]piperidine-2,6-dione (B-319, 130 mg, 243.71 μmol, 92% yield, TFA salt) was synthesized from tert-butyl 6-[1-(2,6-dioxo-3-piperidyl)-3-methyl-2-oxo-benzimidazol-5-yl]-2,6-diazaspiro[3.3]heptane-2-carboxylate (B-318) in a similar fashion to Compound A-62, except using 10 eq. TFA. LCMS (ES+): m/z 356.2 [M+H]⁺.

### 3-[5-(2,6-diazaspiro[3.5]nonan-6-yl)-3-methyl-2-oxo-benzimidazol-1-yl]piperidine-2,6-dione (B-323)

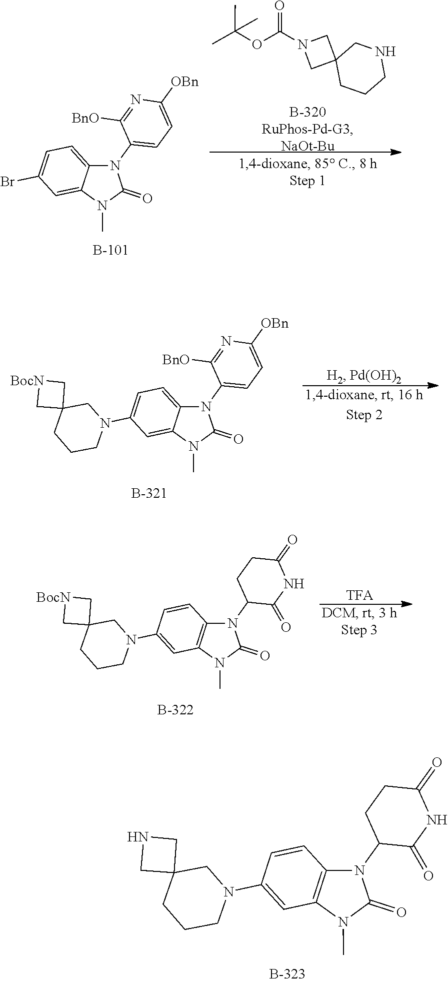 [SMILES: Cn1c(=O)n(-c2ccc(OCc3ccccc3)nc2OCc2ccccc2)c2ccc(Br)cc21 ; CC(C)(C)OC(=O)N1CC2(CCCNC2)C1 ; Cn1c(=O)n(-c2ccc(OCc3ccccc3)nc2OCc2ccccc2)c2ccc(N3CCCC4(CN(C(=O)OC(C)(C)C)C4)C3)cc21 ; Cn1c(=O)n(C2CCC(=O)NC2=O)c2ccc(N3CCCC4(CN(C(=O)OC(C)(C)C)C4)C3)cc21 ; Cn1c(=O)n(C2CCC(=O)NC2=O)c2ccc(N3CCCC4(CNC4)C3)cc21]

### Step 1: tert-butyl 8-[1-(2,6-dibenzyloxy-3-pyridyl)-3-methyl-2-oxo-benzimidazol-5-yl]-2,8-diazaspiro[3.5]nonane-2-carboxylate (B-321)

tert-butyl 8-[1-(2,6-dibenzyloxy-3-pyridyl)-3-methyl-2-oxo-benzimidazol-5-yl]-2,8-diazaspiro[3.5]nonane-2-carboxylate (B-321, 500 mg, 725.31 μmol, 62% yield) was synthesized from 5-bromo-1-(2,6-dibenzyloxy-3-pyridyl)-3-methyl-benzimidazol-2-one (B-101) and t-butyl 2,8-diazaspiro[3.5]nonane-2-carboxylate (B-320) in a similar fashion to Compound B-244, except using 1.8 eq. B-320 and 0.15 eq. RuPhos-Pd-G3. The reaction mixture was filtered through Celite, washing with ethyl acetate. The solvent was removed, and the residue purified by flash silica gel column chromatography (50-60% Ethyl acetate in pet-ether). LCMS (ES+): m/z 662.3 [M+H]⁺.

### Step 2: tert-butyl 8-[1-(2,6-dioxo-3-piperidyl)-3-methyl-2-oxo-benzimidazol-5-yl]-2,8-diazaspiro[3.5]nonane-2-carboxylate (B-322)

tert-butyl 8-[1-(2,6-dioxo-3-piperidyl)-3-methyl-2-oxo-benzimidazol-5-yl]-2,8-diazaspiro[3.5]nonane-2-carboxylate (B-322, 110 mg, 200.18 μmol, 27% yield) was synthesized from tert-butyl 8-[1-(2,6-dibenzyloxy-3-pyridyl)-3-methyl-2-oxo-benzimidazol-5-yl]-2,8-diazaspiro[3.5]nonane-2-carboxylate (B-321) in a similar fashion to Compound B-103, except using 0.2 eq. palladium hydroxide (20% on carbon). The material was purified by flash silica gel column chromatography (70-80% Ethyl acetate in pet-ether). LCMS (ES+): m/z 484.2 [M+H]⁺.

### Step 3: 3-[5-(2,6-diazaspiro[3.5]nonan-6-yl)-3-methyl-2-oxo-benzimidazol-1-yl]piperidine-2,6-dione (B-323)

3-[5-(2,6-diazaspiro[3.5]nonan-6-yl)-3-methyl-2-oxo-benzimidazol-1-yl]piperidine-2,6-dione (B-323, 90 mg, 155.59 μmol, 92% yield, TFA salt) was synthesized from tert-butyl 8-[1-(2,6-dioxo-3-piperidyl)-3-methyl-2-oxo-benzimidazol-5-yl]-2,8-diazaspiro[3.5]nonane-2-carboxylate (B-322) in a similar fashion to Compound A-62, except using 3 eq. TFA. The material was triturated with MTBE. LCMS (ES+): m/z 383.9 [M+H]⁺.

### 3-[3-methyl-2-oxo-5-(4-oxo-1-piperidyl)benzimidazol-1-yl]piperidine-2,6-dione (B-326)

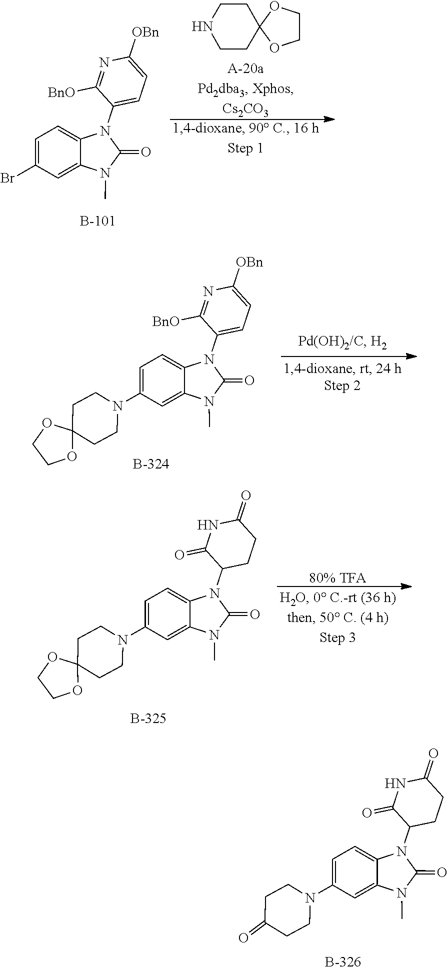 [SMILES: Cn1c(=O)n(-c2ccc(OCc3ccccc3)nc2OCc2ccccc2)c2ccc(Br)cc21 ; C1CC2(CCN1)OCCO2 ; Cn1c(=O)n(-c2ccc(OCc3ccccc3)nc2OCc2ccccc2)c2ccc(N3CCC4(CC3)OCCO4)cc21 ; Cn1c(=O)n(C2CCC(=O)NC2=O)c2ccc(N3CCC4(CC3)OCCO4)cc21 ; Cn1c(=O)n(C2CCC(=O)NC2=O)c2ccc(N3CCC(=O)CC3)cc21]

### Step 1: 1-(2,6-dibenzyloxy-3-pyridyl)-5-(1,4-dioxa-8-azaspiro[4.5]decan-8-yl)-3-methyl-benzimidazol-2-one (B-324)

1-(2,6-dibenzyloxy-3-pyridyl)-5-(1,4-dioxa-8-azaspiro[4.5]decan-8-yl)-3-methyl-benzimidazol-2-one (B-324, 0.6 g, 972.74 μmol, 50% yield) was synthesized from 5-bromo-1-(2,6-dibenzyloxy-3-pyridyl)-3-methyl-benzimidazol-2-one (B-101) and 1,4-dioxa-8-azaspiro[4.5]decane (A-20a) in a similar fashion to Compound B-180, except using 1 eq. cesium carbonate, 1 eq. tris(dibenzylideneacetone) dipalladium (0) and 1 eq. XPhos. LCMS (ES+): m/z 579.31 [M+H]⁺.

### Step 2: 3-[5-(1,4-dioxa-8-azaspiro[4.5]decan-8-yl)-3-methyl-2-oxo-benzimidazol-1-yl]piperidine-2,6-dione (B-325)

3-[5-(1,4-dioxa-8-azaspiro[4.5]decan-8-yl)-3-methyl-2-oxo-benzimidazol-1-yl]piperidine-2,6-dione (B-325, 450 mg, 1.03 mmol, 99% yield) was synthesized from 1-(2,6-dibenzyloxy-3-pyridyl)-5-(1,4-dioxa-8-azaspiro[4,5]decan-8-yl)-3-methyl-benzimidazol-2-one (B-324) in a similar fashion to Compound B-103, except using 0.4 eq. palladium hydroxide (20% on carbon, 50% water). LCMS (ES+): m/z 400.9 [M+H]⁺.

### Step 3: 3-[3-methyl-2-oxo-5-(4-oxo-1-piperidyl)benzimidazol-1-yl]piperidine-2,6-dione (B-326)

Into a 10 mL single neck round bottom flask containing a well-stirred solution of 3-[5-(1,4-dioxa-8-azaspiro[4.5]decan-8-yl)-3-methyl-2-oxo-benzimidazol-1-yl]piperidine-2,6-dione (B-325, 250 mg, 624.33 μmol) in water (0.6 mL) was added TFA (3.2 mL, 4.74 g) dropwise at 0° C. The reaction mixture was stirred at room temperature for 36 h and then heated to 50° C. for 4 h. The reaction mixture was concentrated under reduced pressure and the residue purified by reverse-phase preparative-HPLC [Purification method: Column: SunFire C18 (9×150 mm) 5.0 μm; Solvent A: 0.1% TFA in water; Solvent B: Acetonitrile) to afford 3-[3-methyl-2-oxo-5-(4-oxo-1-piperidyl)benzimidazol-1-yl]piperidine-2,6-dione (B-326, 90 mg, 214.37 μmol, 34% yield) as a brown oil. LCMS (ES+): m/z 356.9 [M+H]⁺.

### 4-[[1-(2,6-dioxo-3-piperidyl)-3-methyl-2-oxo-benzimidazol-5-yl]amino]benzoic acid (B-330)

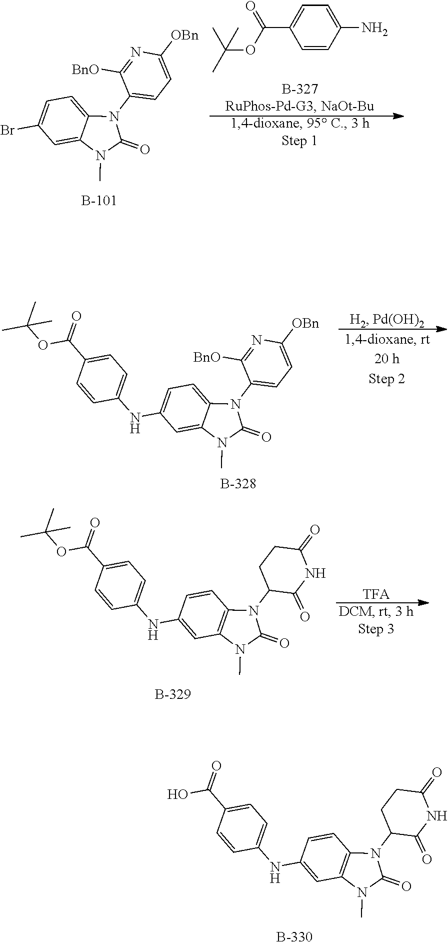 [SMILES: Cn1c(=O)n(-c2ccc(OCc3ccccc3)nc2OCc2ccccc2)c2ccc(Br)cc21 ; CC(C)(C)OC(=O)c1ccc(N)cc1 ; Cn1c(=O)n(-c2ccc(OCc3ccccc3)nc2OCc2ccccc2)c2ccc(Nc3ccc(C(=O)OC(C)(C)C)cc3)cc21 ; Cn1c(=O)n(C2CCC(=O)NC2=O)c2ccc(Nc3ccc(C(=O)OC(C)(C)C)cc3)cc21 ; Cn1c(=O)n(C2CCC(=O)NC2=O)c2ccc(Nc3ccc(C(=O)O)cc3)cc21]

### Step 1: tert-butyl 4-[[1-(2,6-dibenzyloxy-3-pyridyl)-3-methyl-2-oxo-benzimidazol-5-yl]amino]benzoate (B-328)

tert-butyl 4-[1-(2,6-dibenzyloxy-3-pyridyl)-3-methyl-2-oxo-benzimidazol-5-yl]amino]benzoate (B-328, 600 mg, 906.61 mol, 59% yield) was synthesized from 5-bromo-1-(2,6-dibenzyloxy-3-pyridyl)-3-methyl-benzimidazol-2-one (B-101) and tert-butyl 4-aminobenzoate (B-327) in a similar fashion to Compound B-244, except using 1.3 eq. B-327 and 0.15 eq. RuPhos-Pd-G3. Upon completion, the reaction mixture was filtered through Celite, washed with ethyl acetate, solvent was removed, and the material purified by flash silica gel column chromatography (35-40% ethyl acetate in pet-ether). LCMS (ES+): m/z 629.2 [M+H]⁺.

### Step 2: tert-butyl 4-[[1-(2,6-dioxo-3-piperidyl)-3-methyl-2-oxo-benzimidazol-5-yl]amino]benzoate (B-329)

tert-butyl 4-[[1-(2,6-dioxo-3-piperidyl)-3-methyl-2-oxo-benzimidazol-5-yl]amino]benzoate (B-329, 180 mg, 359.61 μmol, 75% yield) was synthesized from tert-butyl 4-[[1-(2,6-dibenzyloxy-3-pyridyl)-3-methyl-2-oxo-benzimidazol-5-yl]amino]benzoate (B-328) in a similar fashion to Compound B-103, except using 0.6 eq. palladium hydroxide (20% on carbon). LCMS (ES+): m/z 451.2 [M+H]⁺.

### Step 3: 4-[[1-(2,6-dioxo-3-piperidyl)-3-methyl-2-oxo-benzimidazol-5-yl]amino]benzoic acid (B-330)

4-[[1-(2,6-dioxo-3-piperidyl)-3-methyl-2-oxo-benzimidazol-5-yl]amino]benzoic acid (B-330, 130 mg, 250.59 μmol, 87% yield, TFA salt) was synthesized from tert-butyl 4-[[1-(2,6-dioxo-3-piperidyl)-3-methyl-2-oxo-benzimidazol-5-yl]amino]benzoate (B-329) in a similar fashion to Compound A-26, except using 10 eq. TFA. LCMS (ES−): m/z 393.0 [M−H]⁻.

### 3-(5-(4-aminophenyl)-3-methyl-2-oxo-2,3-dihydro-1H-benzo[d]imidazol-1-yl)piperidine-2,6-dione (B-333)

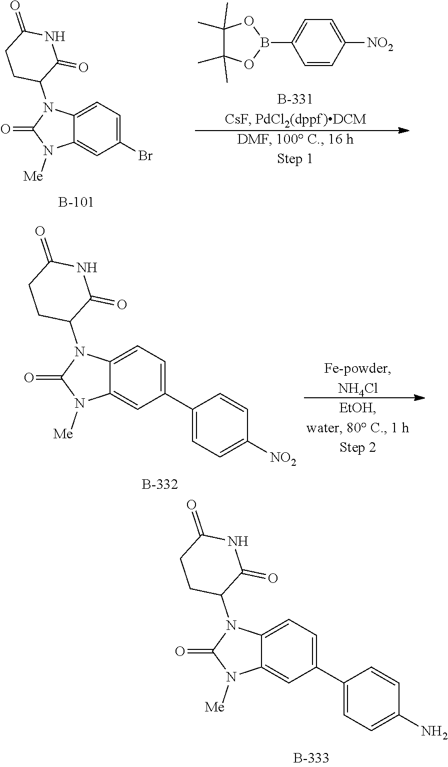 [SMILES: Cn1c(=O)n(C2CCC(=O)NC2=O)c2ccc(Br)cc21 ; CC1(C)OB(c2ccc([N+](=O)[O-])cc2)OC1(C)C ; Cn1c(=O)n(C2CCC(=O)NC2=O)c2ccc(-c3ccc([N+](=O)[O-])cc3)cc21 ; Cn1c(=O)n(C2CCC(=O)NC2=O)c2ccc(-c3ccc(N)cc3)cc21]

### Step 1: 3-(3-methyl-5-(4-nitrophenyl)-2-oxo-2,3-dihydro-1H-benzo[d]imidazol-1-yl)piperidine-2,6-dione (B-332)

3-(3-methyl-5-(4-nitrophenyl)-2-oxo-2,3-dihydro-1H-benzo[d]imidazol-1-yl)piperidine-2,6-dione (B-332, 400 mg, 58% yield) was synthesized from 3-(5-bromo-3-methyl-2-oxo-benzimidazol-1-yl)piperidine-2,6-dione (B-101) and 4,4,5,5-tetramethyl-2-(4-nitrophenyl)-1,3,2-dioxaborolane (B-331) in a similar fashion to Compound B-133, except using 2 eq. B-331, 1.5 eq. CsF and heated at 100° C. for 16 h. The mixture was filtered through Celite, concentrated and purified by silica gel column chromatography (50-60% Ethyl acetate in pet-ether). LCMS (ES+): m/z 381.0 [M+H]⁺.

### Step 2: 3-(5-(4-aminophenyl)-3-methyl-2-oxo-2,3-dihydro-1H-benzo[d]imidazol-1-yl)piperidine-2,6-dione (B-333)

3-(5-(4-aminophenyl)-3-methyl-2-oxo-2,3-dihydro-1H-benzo[d]imidazol-1-yl)piperidine-2,6-dione (B-333, 180 mg, 40% yield) was synthesized from 3-(3-methyl-5-(4-nitrophenyl)-2-oxo-2,3-dihydro-1H-benzo[d]imidazol-1-yl)piperidine-2,6-dione (B-332) in a similar fashion to Compound B-99, except using 5 eq. iron powder and 5 eq. ammonium chloride. Upon completion, the reaction was diluted with ethyl acetate, filtered through Celite and washed with ethyl acetate (50 mL). The filtrate was evaporated concentrated and the residue was diluted with water and extracted with Ethyl acetate (2×). The combined organic layer was washed with brine, dried over sodium sulfate, filtered and concentrated. The material was used in the next step without further purification. LCMS (ES+): m/z 351.0 [M+H]⁺.

### 3-[5-(3,3-difluoro-4-piperidyl)-3-methyl-2-oxo-benzimidazol-1-yl]piperidine-2,6-dione (B-339)

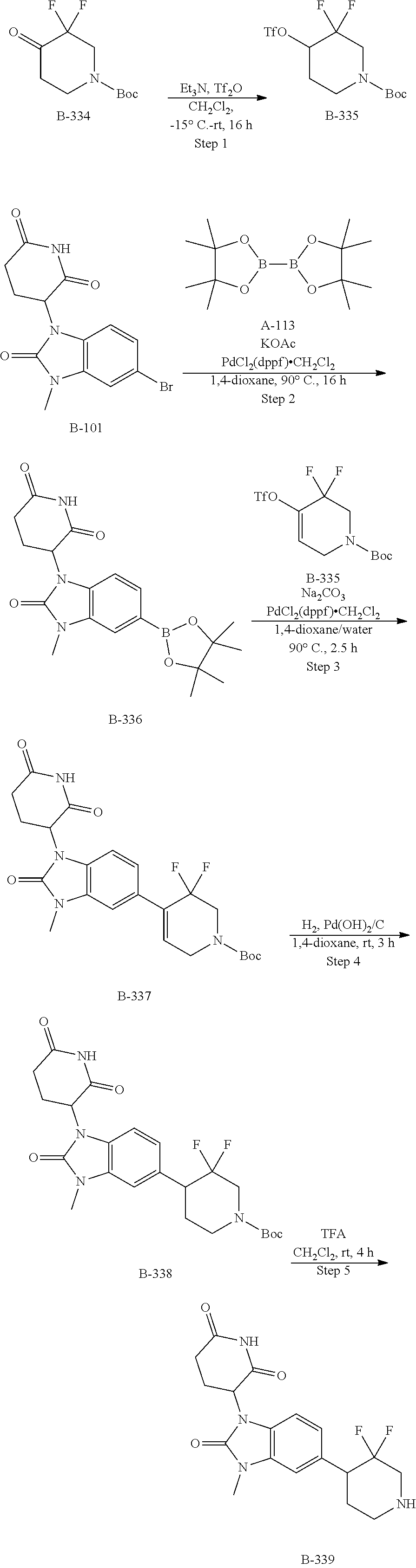

### Step 1: tert-butyl 3,3-difluoro-4-(trifluoromethylsulfonyloxy)-2,6-dihydropyridine-1-carboxylate (B-335)

Into a 250 mL two neck round bottom flask containing a well-stirred solution of tert-butyl 3,3-difluoro-4-oxo-piperidine-1-carboxylate (B-335, 2.5 g, 10.63 mmol) in anhydrous DCM (30 mL) under nitrogen atmosphere at −15° C. were added Et₃N (3.27 g, 32.29 mmol, 4.5 mL) and trifluoromethanesulfonic anhydride (4.50 g, 15.96 mmol, 2.68 mL) via dropwise addition. The reaction mixture was stirred at room temperature for 16 h. The reaction was quenched with aqueous sodium bicarbonate and extracted with DCM (2×50 mL). The organic layers were combined, washed with brine, dried over sodium sulfate, filtered and concentrated. The residue was purified by flash silica gel column chromatography (25% Ethyl acetate in pet-ether) to afford tert-butyl 3,3-difluoro-4-(trifluoromethylsulfonyloxy)-2,6-dihydropyridine-1-carboxy late (B-335, 1.3 g, 3.54 mmol, 33% yield) as a yellow solid.

### Step 2: 3-[3-methyl-2-oxo-5-(4,4,5,5-tetramethyl-1,3,2-dioxaborolan-2-yl)benzimidazol-1-yl]piperidine-2,6-dione (B-336)

Into a 100 mL pressure tube containing a well-stirred solution of 3-(5-bromo-3-methyl-2-oxo-benzimidazol-1-yl)piperidine-2,6-dione (B-101, 4 g, 11.83 mmol) in 1,4-dioxane (40 mL) was added 4,4,4′,4′,5,5,5′,5′-octamethyl-2,2′-bi(1,3,2-dioxaborolane) (A-113, 3.00 g, 11.83 mmol). The reaction mixture was purged by bubbling nitrogen through the solution for 5 min Pd(dppf)Cl₂·CH₂Cl (965.99 mg, 1.18 mmol) was added and the resulting suspension was heated at 90° C. for 16 h. The reaction mixture was cooled to room temperature, filtered through Celite, washing with acetonitrile (25 mL), and concentrated under reduced pressure. The residue was purified by flash silica-gel column chromatography (0-100% Ethyl acetate in pet-ether) to afford 3-[3-methyl-2-oxo-5-(4,4,5,5-tetramethyl-1,3,2-dioxaborolan-2-yl)benzimidazol-1-yl]piperidine-2,6-dione (B-336, 3.6 g, 7.39 mmol, 62% yield) as an off-white solid. LCMS (ES+): m/z 386.2 [M+H]⁺.

### Step 3: tert-butyl 4-[1-(2,6-dioxo-3-piperidyl)-3-methyl-2-oxo-benzimidazol-5-yl]-3,3-difluoro-2,6-dihydropyridine-1-carboxylate (B-337)

Into a 50 ml pressure tube containing a well-stirred solution of 3-[3-methyl-2-oxo-5-(4,4,5,5-tetramethyl-1,3,2-dioxaborolan-2-yl)benzimidazol-1-yl]piperidine-2,6-dione (B-336, 500 mg, 1.03 mmol) and tert-butyl 3,3-difluoro-4-(trifluoromethylsulfonyloxy)-2,6-dihydropyridine-1-carboxylate (B-335, 423.80 mg, 1.03 mmol) in 1,4-dioxane (2.7 mL) and water (0.2 mL) was added Na₂CO₃ (108.72 mg, 1.03 mmol, 42.97 μL) and the mixture purged with nitrogen gas for 5 min Pd(dppf)Cl₂·CH₂Cl₂ (83.77 mg, 102.58 μmol) was added and the reaction mixture was heated at 90° C. for 2.5 h. The reaction was cooled to room temperature and poured onto water (15 mL). The aqueous layer was extracted with Ethyl acetate (2×40 mL). The organic layers were combined, washed with brine (20 mL), dried over anhydrous sodium sulfate, filtered and concentrated under reduced pressure. The residue was purified by flash silica-gel column chromatography (0-100% Ethyl acetate in pet-ether) to afford tert-butyl 4-[1-(2,6-dioxo-3-piperidyl)-3-methyl-2-oxo-benzimidazol-5-yl]-3,3-difluoro-2,6-dihydropyridine-1-carboxylate (B-337, 520 mg, 801.71 μmol, 78% yield) as a pale yellow solid. LCMS (ES−): m/z 475.0 [M−H]⁺.

### Step 4: tert-butyl 4-[1-(2,6-dioxo-3-piperidyl)-3-methyl-2-oxo-benzimidazol-5-yl]-3,3-difluoro-piperidine-1-carboxylate (B-338)

tert-butyl 4-[1-(2,6-dioxo-3-piperidyl)-3-methyl-2-oxo-benzimidazol-5-yl]-3,3-difluoro-piperidine-1-carboxylate (B-338, 400 mg, 782.05 μmol, 98% yield) was synthesized from tert-butyl 4-[1-(2,6-dioxo-3-piperidyl)-3-methyl-2-oxo-benzimidazol-5-yl]-3,3-difluoro-2,6-dihydropyridine-1-carboxylate (B-337) in a similar fashion to Compound B-103, except using 0.4 eq. palladium hydroxide (20% on carbon, 50% water). The material was triturated with diethyl ether. LCMS (ES−): m/z 477.2 [M−H]⁻. ¹H-NMR (400 MHz, DMSO-d6): δ 11.11 (s, 1H), 7.15 (s, 1H), 7.08 (d, J=8.00 Hz, 1H), 6.97 (d, J=8.40 Hz, 1H), 5.37 (dd, J=5.60, 12.60 Hz, 1H), 4.38-4.01 (m, 2H), 2.96-2.87 (m, 2H), 2.76-2.50 (m, 4H), 1.75-2.15 (m, 3H), 1.43 (s, 9H).

### Step 5: 3-[5-(3,3-difluoro-4-piperidyl)-3-methyl-2-oxo-benzimidazol-1-yl]piperidine-2,6-dione (B-339)

3-[5-(3,3-difluoro-4-piperidyl)-3-methyl-2-oxo-benzimidazol-1-yl]piperidine-2,6-dione (B-339, 390 mg, 742.70 μmol, 99% yield, TFA salt) was synthesized from tert-butyl4-[1-(2,6-dioxo-3-piperidyl)-3-methyl-2-oxo-benzimidazol-5-yl]-3,3-difluoro-piperidine-1-carboxylate (B-338) in a similar fashion to Compound A-62, except using 10 eq. TFA. LCMS (ES+): m/z 379.2 [M+H]⁺. ¹H NMR (400 MHz, DMSO-d6): δ 11.13 (s, 1H), 9.38 (br s, 1H), 7.12 (d, J=8.00 Hz, 1H), 7.05 (s, 1H), 6.97 (d, J=8.00 Hz, 1H), 5.39 (dd, J=5.60, 13.00 Hz, 1H), 3.82 (t, J=8.40 Hz, 1H), 3.61-3.49 (m, 2H), 3.36-3.32 (m, 1H), 2.93-2.85 (m, 1H), 2.80-2.60 (m, 3H), 2.34-2.28 (m, 1H), 2.11-2.01 (m, 2H).

### 3-[4-fluoro-3-methyl-2-oxo-5-(4-piperidyl)benzimidazol-1-yl]piperidine-2,6-dione (B-347)

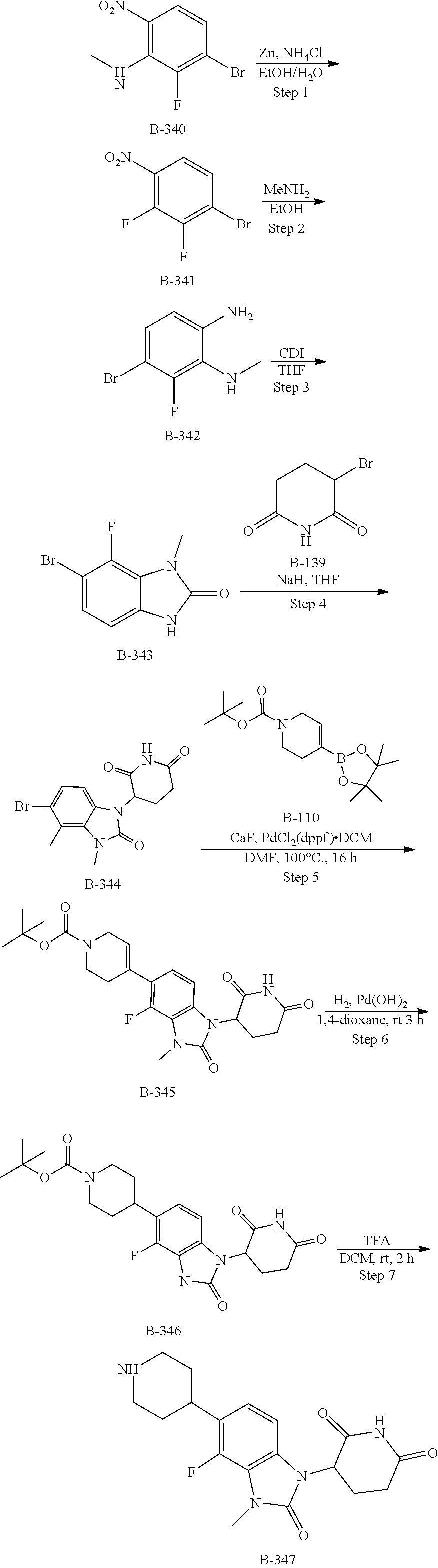

### Step 1: 4-bromo-3-fluoro-N2-methyl-benzene-1,2-diamine (B-341)

4-bromo-3-fluoro-N2-methyl-benzene-1,2-diamine (B-341, 2.4 g, 6.57 mmol, 32.74% yield) was synthesized from 3-bromo-2-fluoro-N-methyl-6-nitro-aniline (B-340) in a similar fashion to Compound A-18a/b, except following filtration, the material was purified by silica gel column chromatography, eluting with 15% Ethyl acetate and Hexane.

### Step 2: 3-bromo-2-fluoro-N-methyl-6-nitro-aniline (B-342)

A solution of 1-bromo-2,3-difluoro-4-nitro-benzene (B-341, 5 g, 21.01 mmol) in ethanol (30 mL) was treated with methyl amine (10.25 g, 23.11 mmol, 11.41 mL, 7% purity). After 3 h, the reaction mixture was concentrated under reduced pressure. The residue was dissolved in ethyl acetate and washed with water and brine solution. The organic layer was dried over sodium sulfate, filtered and concentrated under reduced pressure. The residue was purified by silica gel column chromatography to afford 3-bromo-2-fluoro-N-methyl-6-nitro-aniline (B-342, 4.4 g, 16.78 mmol, 80% yield). LCMS (ES+): m/z 249.0 [M+H]⁺.

### Step 3: 5-bromo-4-fluoro-3-methyl-1H-benzimidazol-2-one (B-343)

5-bromo-4-fluoro-3-methyl-1H-benzimidazol-2-one (B-343, 1.3 g, 5.04 mmol, 48% yield) was synthesized from 4-bromo-3-fluoro-N2-methyl-benzene-1,2-diamine (B-342) in a similar fashion to Compound B-208, except using 3.2 eq. CDI and heating at 60° C. for 5 h. LCMS (ES+): m/z 246.9 [M+H]⁺.

### Step 4: 3-(5-bromo-4-fluoro-3-methyl-2-oxo-benzimidazol-1-yl)piperidine-2,6-dione (B-344)

3-(5-bromo-4-fluoro-3-methyl-2-oxo-benzimidazol-1-yl)piperidine-2,6-dione (B-344, 500 mg, 1.37 mmol, 34% yield) was synthesized from 5-bromo-4-fluoro-3-methyl-1H-benzimidazol-2-one (B-343) and 3-bromopiperidine-2,6-dione (B-139) in a similar fashion to Compound B-3, except using 5 eq. B-139 and 10 eq. sodium hydride. The reaction was heated to 60° C. for 16 h. The reaction was poured into cold saturated ammonium chloride solution and extracted with ethyl acetate. The organic layer was washed with water and brine, dried over sodium sulfate, filtered and concentrated. The material was purified by silica gel column chromatography eluting with 70% ethyl acetate in hexane. ¹H NMR (400 MHz, DMSO-d6) d 10.82 (bs, 1H), 7.31 (t, J=6.6 Hz, 1H), 7.00 (d, J=8.0 Hz, 1H), 5.42-5.38 (m, 1H), 3.49 (s, 3H), 2.92-2.83 (m, 1H), 2.74-2.60 (m, 2H), 2.07-2.01 (m, 1H).

### Step 5: t-butyl 4-[1-(2,6-dioxo-3-piperidyl)-4-fluoro-3-methyl-2-oxo-benzimidazol-5-yl]-3,6-dibydro-2H-pyridine-1-carboxylate (B-345)

t-butyl 4-[1-(2,6-dioxo-3-piperidyl)-4-fluoro-3-methyl-2-oxo-benzimidazol-5-yl]-3,6-dihydro-2H-pyridine-1-carboxylate (B-345, 200 mg, 388.24 μmol, 69% yield) was synthesized from 3-(5-bromo-4-fluoro-3-methyl-2-oxo-benzimidazol-1-yl)piperidine-2,6-dione (B-344) and t-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 2.5 eq. B-110, 4 eq. CsF and 0.2 eq. [1,1′-Bis(diphenylphosphino)ferrocene]dichloropalladium(II) complex with dichloromethane. The reaction mixture was heated at 100° C. for 16 h. The reaction mixture was filtered through Celite, washing with Ethyl acetate, the solvent removed and the residue purified by flash silica gel column chromatography (40-50% Ethyl acetate in pet-ether). LCMS (ES−): m/z 457.2 [M−H]⁻.

### Step 6: t-butyl 4-[1-(2,6-dioxo-3-piperidyl)-4-fluoro-3-methyl-2-oxo-benzimidazol-5-yl]piperidine-1-carboxylate (B-346)

t-butyl 4-[1-(2,6-dioxo-3-piperidyl)-4-fluoro-3-methyl-2-oxo-benzimidazol-5-yl]piperidine-1-carboxylate (B-346, 170 mg, 313.79 μmol, 81% yield) was synthesized from t-butyl 4-[1-(2,6-dioxo-3-piperidyl)-4-fluoro-3-methyl-2-oxo-benzimidazol-5-yl]-3,6-dihydro-2H-pyridine-1-carboxylate (B-345) in a similar fashion to Compound B-103, except using 0.2 eq. palladium hydroxide (20% on carbon). LCMS (ES−): m/z 459.2 [M−H]⁻.

### Step 7: 3-[4-fluoro-3-methyl-2-oxo-5-(4-piperidyl)benzimidazol-1-yl]piperidine-2,6-dione (B-347)

3-[4-fluoro-3-methyl-2-oxo-5-(4-piperidyl)benzimidazol-1-yl]piperidine-2,6-dione (B-347, 140 mg, 247.89 μmol, 79% yield, TFA salt) was synthesized from t-butyl 4-[1-(2,6-dioxo-3-piperidyl)-4-fluoro-3-methyl-2-oxo-benzimidazol-5-yl]piperidine-1-carboxylate (B-346) in a similar fashion to Compound A-62, except using 3 eq. TFA. LCMS (ES+): 361.2 [M+H]⁺.

### 3-(6-fluoro-3-methyl-2-oxo-5-(piperidin-4-yl)-2,3-dihydro-1H-benzo[d]imidazol-1-yl)piperidine-2,6-dione (B-355)

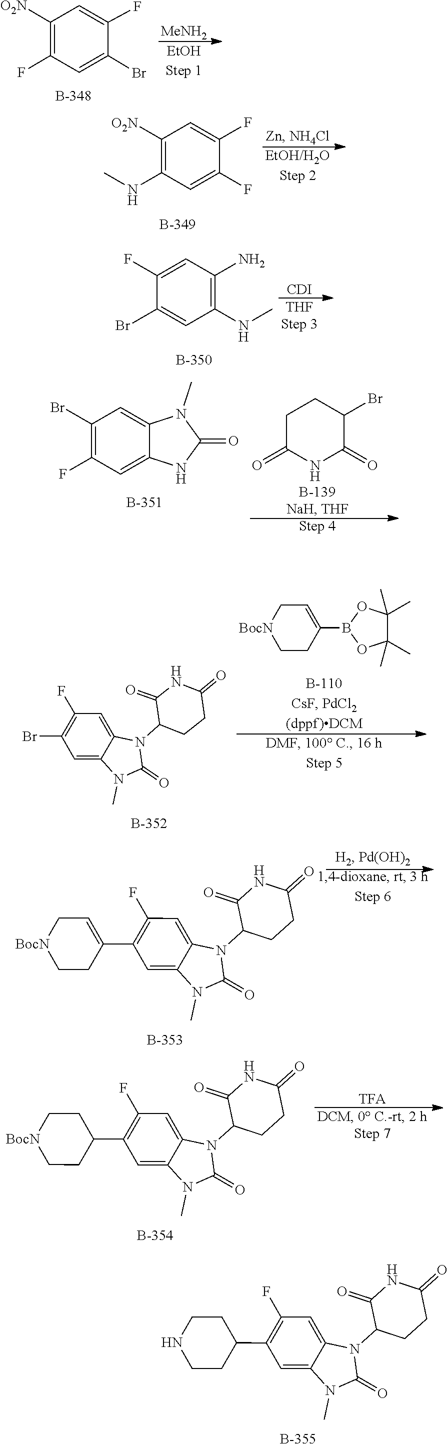

### Step 1: 5-bromo-4-fluoro-N-methyl-2-nitro-aniline (B-349)

5-bromo-4-fluoro-N-methyl-2-nitro-aniline (B-349, 3.35 g, 12.51 mmol, 60% yield) was synthesized from 1-bromo-2,5-difluoro-4-nitro-benzene (B-348) in a similar fashion to Compound B-342.

### Step 2: 5-bromo-4-fluoro-N1-methylbenzene-1,2-diamine (B-350)

4-bromo-5-fluoro-N1-methylbenzene-1,2-diamine (B-350, 5 g, 21.91 mmol, 81% yield) was synthesized from 5-bromo-4-fluoro-N-methyl-2-nitro-aniline (B-349) in a similar fashion to Compound A-18a/b, except upon filtration, the residue was purified by silica gel column chromatography, eluting with 15% ethyl acetate and hexanes. LCMS (ES+): m/z 219.2/221.1 [M+H]⁺.

### Step 3: 6-bromo-5-fluoro-1-methyl-1,3-dihydro-1H-benzo[d]imidazol-2-one (B-351)

6-bromo-5-fluoro-1-methyl-1,3-dihydro-1H-benzo[d]imidazol-2-one (B-351, 3.5 g, 12.14 mmol, 48% yield) was synthesized from 4-bromo-5-fluoro-N1-methyl-benzene-1,2-diamine (B-350) in a similar fashion to Compound B-208, except using 2 eq. CDI and heating at 55° C. for 5 h. Upon completion the mixture was concentrated and the residue purified by silica gel column chromatography. LCMS (ES+): m/z 254.2/247.1 [M+H]⁺.

### Step 4: 3-(5-bromo-6-fluoro-3-methyl-2-oxo-2,3-dihydro-1H-benzo[d]imidazol-1-yl)piperidine-2,6-dione (B-352)

3-(5-bromo-6-fluoro-3-methyl-2-oxo-2,3-dihydro-1H-benzo[d]imidazol-1-yl)piperidine-2,6-dione (B-352, 1.2 g, 3.32 mmol, 37% yield) was synthesized from 6-bromo-5-fluoro-1-methyl-1,3-dihydro-1H-benzo[d]imidazol-2-one (B-351) and 3-bromopiperidine-2,6-dione (B-139) in a similar fashion to Compound B-3, except using 9 eq. sodium hydride and 5 eq. B-139. The reaction was heated at 70° C. for 4 h. The reaction mixture was quenched with ice water and extracted with ethyl acetate. The material was purified by silica gel column chromatography. LCMS (ES−): m/z 354.0 [M−H]⁻.

### Step 5: tert-butyl 4-(1-(2,6-dioxopiperidin-3-yl)-6-fluoro-3-methyl-2-oxo-2,3-dihydro-1H-benzo[d]imidazol-5-yl)-3,6-dihydropyridine-1(2H)-carboxylate (B-353)

tert-butyl 4-(1-(2,6-dioxopiperidin-3-yl)-6-fluoro-3-methyl-2-oxo-2,3-dihydro-1H-benzo[d]imidazol-5-yl)-3,6-dihydropyridine-1(2H)-carboxylate (B-353, 120 mg, 205.41 μmol, 41% yield) was synthesized from 3-(5-bromo-6-fluoro-3-methyl-2-oxo-2,3˜dihydro-1H-benzo[d]imidazol-1-yl)piperidine-2,6-dione (B-352) and tert-butyl 4-(4,4,5,5-tetramethyl-1,3,2-dioxaborolan-2-yl)-3,6-dihydro-2H-pyridine-1-carboxy late (B-110) in a similar fashion to Compound B-133, except using 1.5 eq. of B-110 and 0.15 eq. of [1,1′-Bis(diphenylphosphino)ferrocene]dichloropalladium(II) complex with dichloromethane. The reaction mixture was heated at 100° C. for 16 h. The reaction mixture was filtered through Celite, washing with ethyl acetate. The filtrate was concentrated under reduced pressure and the residue was taken up in Ethyl acetate and washed with water (3×). The organic layer was dried over sodium sulfate, filtered and concentrated. The material was purified by flash silica gel column chromatography (70% Ethyl acetate in petroleum ether). LCMS (ES+): m/z 359.1 [M−Boc+H]⁺.

### Step 6: tert-butyl 4-[1-(2,6-dioxo-3-piperidyl)-6-fluoro-3-methyl-2-oxo-benzimidazol-5-yl]piperidine-1-carboxylate (B-354)

tert-butyl 4-[1-(2,6-dioxo-3-piperidyl)-6-fluoro-3-methyl-2-oxo-benzimidazol-5-yl]piperidine-1-carboxylate (B-354, 100 mg, 182.11 μmol, 83% yield) was synthesized from tert-butyl 4-(1-(2,6-dioxopiperidin-3-yl)-6-fluoro-3-methyl-2-oxo-2,3-dihydro-1H-benzo[d]imidazol-5-yl)-3,6-dihydropyridine-1(2H)-carboxylate (B-353) in a similar fashion to Compound B-103, except using 0.2 eq. palladium hydroxide (20% on carbon). LCMS (ES+): m/z 405.1 [M−tBu+H]⁺.

### Step 7: 3-(6-fluoro-3-methyl-2-oxo-5-(piperidin-4-yl)-2,3-dihydro-1H-benzo[d]imidazol-1-yl)piperidine-2,6-dione (B-355)

3-(6-fluoro-3-methyl-2-oxo-5-(piperidin-4-yl)-2,3-dihydro-1H-benzo[d]imidazol-1-yl)piperidine-2,6-dione (B-355, 120 mg, 206.61 μmol, 79% yield, TFA salt) was synthesized from tert-butyl 4-[1-(2,6-dioxo-3-piperidyl)-6-fluoro-3-methyl-2-oxo-benzimidazol-5-yl]piperidine-1-carboxylate (B-354) in a similar fashion to Compound A-62, except using 10 eq. TFA. LCMS (ES+): m/z 361.1 [M+H]⁺.

### 3-[3-cyclopropyl-2-oxo-5-(4-piperidyl)benzimidazol-1-yl]piperidine-2,6-dione (B-363)

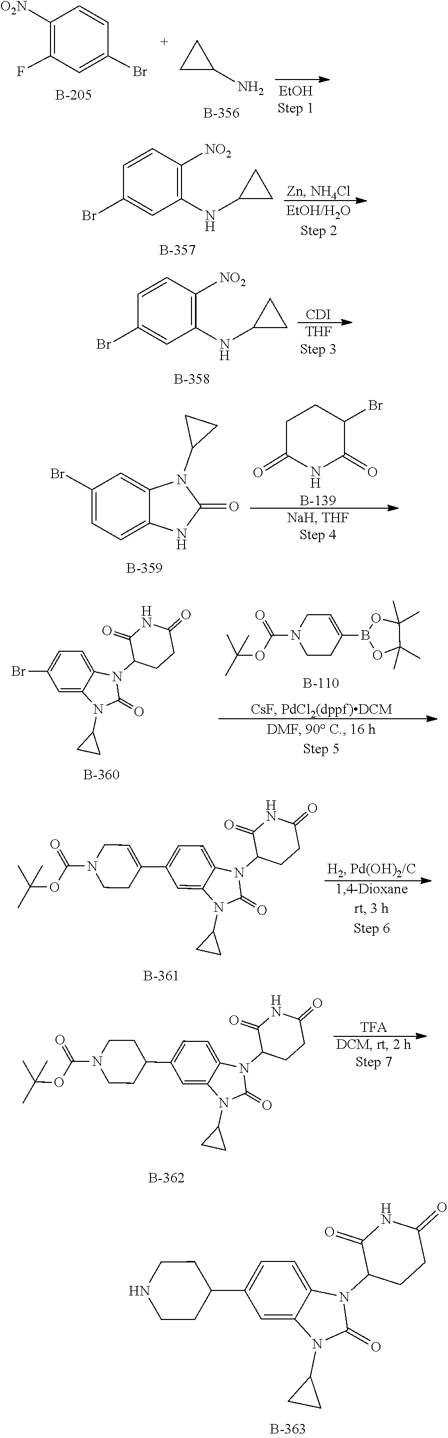

### Step 1: 5-bromo-N-cyclopropyl-2-nitroaniline (B-357)

5-bromo-N-cyclopropyl-2-nitroaniline (B-357, 6 g, 23.11 mmol, 85% yield) was synthesized from 4-bromo-2-fluoro-1-nitrobenzene (B-205) and cyclopropanamine (B-356) in a similar fashion to Compound B-342.

### Step 2: 4-bromo-N2-cyclopropyl-benzene-1,2-diamine (B-358)

4-bromo-N2-cyclopropyl-benzene-1,2-diamine (B-358, 4.34 g, 15.86 mmol, 68% yield) was synthesized from 5-bromo-N-cyclopropyl-2-nitroaniline (B-357) in a similar fashion to Compound A-18a/b, except upon filtration, the material was purified by silica gel column chromatography. LCMS (ES+): m/z 227.3/229.3 [M+H]⁺.

### Step 3: 6-bromo-1-cyclopropyl-1,3-dihydro-2H-benzo[d]imidazol-2-one (B-359)

6-bromo-1-cyclopropyl-1,3-dihydro-2H-benzo[d]imidazol-2-one (B-359, 2.3 g, 8.09 mmol, 46% yield) was synthesized from 4-bromo-N2-cyclopropyl-benzene-1,2-diamine (B-358) in a similar fashion to Compound B-208, except using 2 eq. of CDI and heating at 55° C. for 5 h. Upon completion, the mixture was concentrated and the residue purified by silica gel column chromatography. LCMS (ES+): m/z 253.2/255.2 [M+H]⁺.

### Step 4: 3-(5-bromo-3-cyclopropyl-2-oxo-benzimidazol-1-yl)piperidine-2,6-dione (B-360)

3-(5-bromo-3-cyclopropyl-2-oxo-benzimidazol-1-yl)piperidine-2,6-dione (B-360, 920 mg, 2.48 mmol, 48% yield) was synthesized from 6-bromo-1-cyclopropyl-1,3-dihydro-2H-benzo[d]imidazol-2-one (B-359) and 3-bromopiperidine-2,6-dione (B-139) in a similar fashion to Compound B-3, except using 5 eq. B-139 and 10 eq. sodium hydride. The reaction was heated at 60° C. for 3 h. The reaction mixture was quenched with crushed ice and extracted with ethyl acetate. The organic layer was washed with water and brine, dried over sodium sulfate, filtered and concentrated. The material was purified by silica gel column chromatography (50-70% ethyl acetate in hexane). ¹H NMR (400 MHz, DMSO-d₆) δ 11.10 (s, 1H), 7.39 (s, 1H), 7.22 (d, J=8.3 Hz, 1H), 7.08 (d, J=8.4 Hz, 1H), 7.34-7.32 (m, 1H), 2.91-2.83 (m, 2H), 2.70-2.69 (m, 2H), 1.99 (d, J=8.0 Hz, 1H), 1.04 (d, J=5.8 Hz, 2H), 0.89 (s, 2H).

### Step 5: tert-butyl 4-[3-cyclopropyl-1-(2,6-dioxo-3-piperidyl)-2-oxo-benzimidazol-5-yl]-3,6-dihydro-2H-pyridine-1-carboxylate (B-361)

tert-butyl4-[3-cyclopropyl-1-(2,6-dioxo-3-piperidyl)-2-oxo-benzimidazol-5-yl]-3,6-dihydro-2H-pyridine-1-carboxy late (B-361, 34 mg, 61.14 μmol, 22% yield) was synthesized from 3-(5-bromo-3-cyclopropyl-2-oxo-benzimidazol-1-yl)piperidine-2,6-dione (B-360) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 3 eq. B-110, 5 eq. CsF and 0.2 eq. [1,1′-Bis(diphenylphosphino)ferrocene]dichloropalladium(II) complex with dichloromethane. Upon completion, the reaction mixture was filtered through Celite, washing with ethyl acetate. The filtrate was concentrated under reduced pressure and the material purified by flash silica gel column chromatography (80-100% Ethyl acetate in petroleum ether). LCMS (ES+): m/z 467.2 [M+H]⁺.

### Step 6: tert-butyl 4-[3-cyclopropyl-1-(2,6-dioxo-3-piperidyl)-2-oxo-benzimidazol-5-yl]piperidine-1-carboxylate (B-362)

tert-butyl 4-[3-cyclopropyl-1-(2,6-dioxo-3-piperidyl)-2-oxo-benzimidazol-5-yl]piperidine-1-carboxylate (B-362, 73 mg, 130.59 μmol, 77% yield) was synthesized from tert-butyl 4-[3-cyclopropyl-1-(2,6-dioxo-3-piperidyl)-2-oxo-benzimidazol-5-yl]-3,6-dihydro-2H-pyridine-1-carboxylate (B-361) in a similar fashion to Compound B-103, except using 0.4 eq. palladium hydroxide (20% on carbon). LCMS (ES+):

m/z 413.2 [M−tBu+H]⁺.

### Step 7: 3-[3-cyclopropyl-2-oxo-5-(4-piperidyl)benzimidazol-1-yl]piperidine-2,6-dione (B-363)

3-[3-cyclopropyl-2-oxo-5-(4-piperidyl)benzimidazol-1-yl]piperidine-2,6-dione (B-363, 74 mg, 101.96 μmol, 60% yield, TFA salt) was synthesized from tert-butyl 4-[3-cyclopropyl-1-(2,6-dioxo-3-piperidyl)-2-oxo-benzimidazol-5-yl]piperidine-1-carboxylate (B-362) in a similar fashion to Compound A-62, except using 6 eq. TFA. LCMS (ES+): m/z 369.2 [M+H]⁺.

### 3-[2-oxo-5-(4-piperidyl)-3-(2,2,2-trifluoroethyl)benzimidazol-1-yl]piperidine-2,6-dione (B-371)

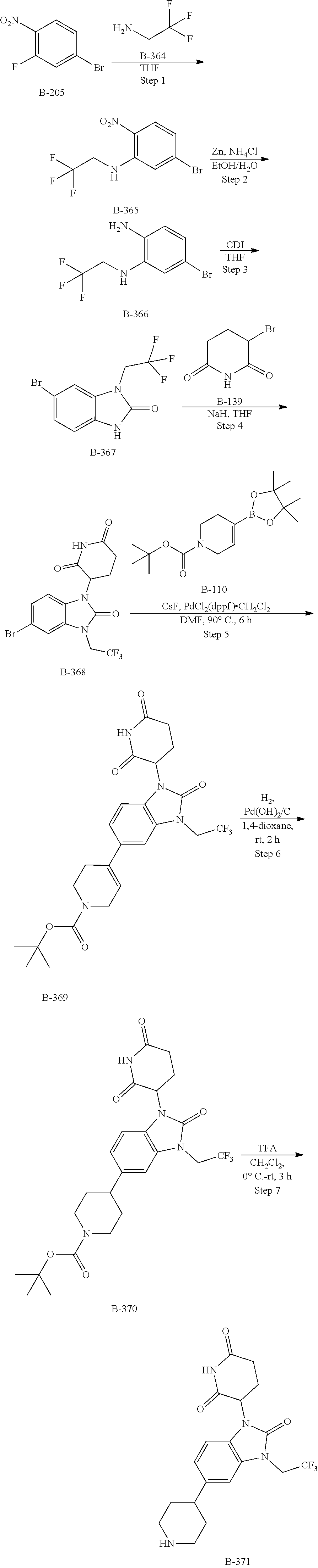

### Step 1: 5-bromo-2-nitro-N-(2,2,2-trifluoroethyl)aniline (B-365)

To the solution of 4-bromo-2-fluoro-1-nitrobenzene (B-205, 5 g, 22.73 mmol) in THF (50 mL) was added slowly N-ethyl-N-isopropyl-propan-2-amine (B-364, 8.81 g, 68.18 mmol, 11.88 mL) at 0° C., followed by 2,2,2-trifluoroethanamine (4.50 g, 45.46 mmol, 3.60 mL). The reaction was heated at 70° C. for 16 h. The solvent was removed and the residue dissolved in ethyl acetate and washed with water and brine solution. The organic layer was dried over sodium sulfate, filtered and concentrated. The residue was purified by silica gel column chromatography to afford 5-bromo-2-nitro-N-(2,2,2-trifluoroethyl)aniline (B-365, 4.8 g, 15.73 mmol, 69% yield).

### Step 2: 4-bromo-N2-(2,2,2-trifluoroethyl)benzene-1,2-diamine (B-366)

4-bromo-N2-(2,2,2-trifluoroethyl)benzene-1,2-diamine (B-366, 3.25 g, 11.96 mmol, 73% yield) was synthesized from 5-bromo-2-nitro-N-(2,2,2-trifluoroethyl)aniline (B-365) in a similar fashion to Compound A-18a/b, except upon filtration, the material was purified by silica gel column chromatography, eluting with 15% Ethyl acetate and Hexane.

### Step 3: 5-bromo-3-(2,2,2-trifluoroethyl)-1H-benzimidazol-2-one (B-367)

5-bromo-3-(2,2,2-trifluoroethyl)-1H-benzimidazol-2-one (B-367, 340 mg, 1.14 mmol, 61% yield) was synthesized from 4-bromo-N2-(2,2,2-trifluoroethyl)benzene-1,2-diamine (B-366) in a similar fashion to Compound B-208, except using 1.2 eq. CDI and heating at 55° C. for 5 h. Upon completion, the mixture was concentrated and the material purified by silica gel column chromatography. LCMS (ES−): m/z 293.2 [M−H]⁻.

### Step 4: 3-[5-bromo-2-oxo-3-(2,2,2-trifluoroethyl)benzimidazol-1-yl]piperidine-2,6-dione (B-368)

3-[5-bromo-2-oxo-3-(2,2,2-trifluoroethyl)benzimidazol-1-yl]piperidine-2,6-dione (B-368, 1.05 g, 2.37 mmol, 39% yield) was synthesized from 5-bromo-3-(2,2,2-trifluoroethyl)-1H-benzimidazol-2-one (B-367) and 3-bromopiperidine-2,6-dione (B-139) in a similar fashion to Compound B-3, except using 10 eq. NaH and 5 eq. B-139. The reaction was heated at 60° C. for 16 h. The reaction was quenched with crushed ice and extracted with ethyl acetate. The organic layer was washed with water and brine, dried over sodium sulfate, filtered and concentrated. The material was purified by column chromatography, eluting with 70% ethyl acetate in hexane. LCMS (ES−): m/z 404.0 [M−H]⁻.

### Step 5: tert-butyl 4-[1-(2,6-dioxo-3-piperidyl)-2-oxo-3-(2,2,2-trifluoroethyl)benzimidazol-5-yl]-3,6-dihydro-2H-pyridine-1-carboxylate (B-369)

tert-butyl 4-[1-(2,6-dioxo-3-piperidyl)-2-oxo-3-(2,2,2-trifluoroethyl)benzimidazol-5-yl]-3,6-dihydro-2H-pyridine-1-carboxylate (B-369, 90 mg, 148.68 μmol, 60% yield) was synthesized from 3-[5-bromo-2-oxo-3-(2,2,2-trifluoroethyl)benzimidazol-1-yl]piperidine-2,6-dione (B-368) and tert-butyl 4-(4,4,5,5-tetramethyl-1,3,2-dioxaborolan-2-yl)-3,6-dihydro-2H-pyridine-1-carboxylate (B-110) in a similar fashion to Compound B-133, except using 2 eq. of B-110 and 4 eq. of CsF. Upon completion, the mixture was filtered through Celite, washing with Ethyl acetate. The filtrate was concentrated and the material purified by flash silica gel column chromatography (70% Ethyl acetate in petroleum ether). LCMS (ES−): m/z 507.2 [M−H]⁻.

### Step 6: tert-butyl 4-[1-(2,6-dioxo-3-piperidyl)-2-oxo-3-(2,2,2-trifluoroethyl)benzimidazol-5-yl]piperidine-1-carboxylate (B-370)

tert-butyl 4-[1-(2,6-dioxo-3-piperidyl)-2-oxo-3-(2,2,2-trifluoroethyl)benzimidazol-5-yl]piperidine-1-carboxylate (B-370, 90 mg, 135.92 μmol, 91% yield) was synthesized from tert-butyl 4-[1-(2,6-dioxo-3-piperidyl)-2-oxo-3-(2,2,2-trifluoroethyl)benzimidazol-5-yl]-3,6-dihydro-2H-pyridine-1-carboxylate (B-369) in a similar fashion to Compound B-103, except using 0.4 eq. palladium hydroxide (20% on carbon, 50% water). LCMS (ES−): m/z 509.2 [M−H]⁻.

### Step 7: 3-[2-oxo-5-(4-piperidyl)-3-(2,2,2-trifluoroethyl)benzimidazol-1-yl]piperidine-2,6-dione (B-371)

3-[2-oxo-5-(4-piperidyl)-3-(2,2,2-trifluoroethyl)benzimidazol-1-yl]piperidine-2,6-dione (B-371, 70 mg, 120.20 μmol, 89% yield, TFA salt) was synthesized from tert-butyl 4-[1-(2,6-dioxo-3-piperidyl)-2-oxo-3-(2,2,2-trifluoroethyl)benzimidazol-5-yl]piperidine-1-carboxylate (B-370) in a similar fashion to Compound A-62, except using 15 eq. TFA. LCMS (ES+): m/z 411.0 [M+H]⁺.

### 3-[6-(3-aminopropyl)-2-oxo-benzo[cd]indol-1-yl]piperidine-2,6-dione (B-376)

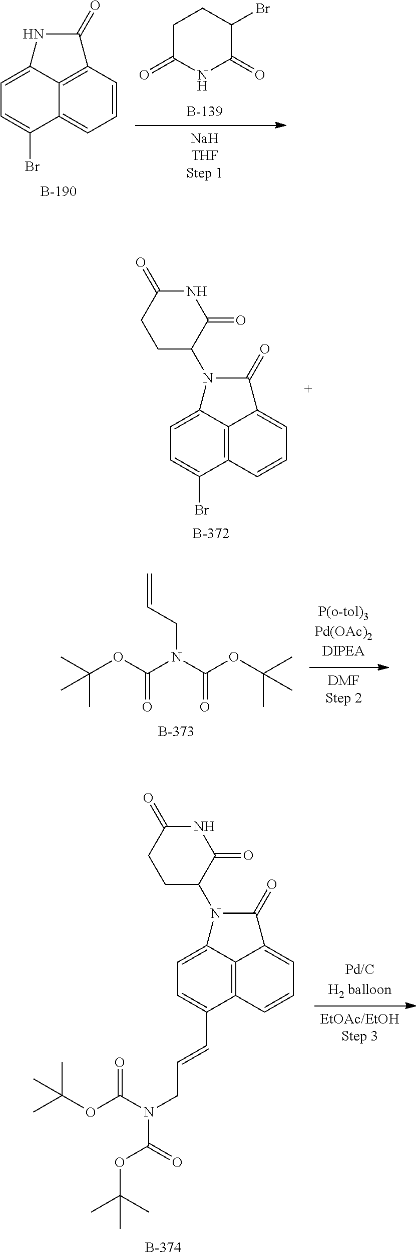

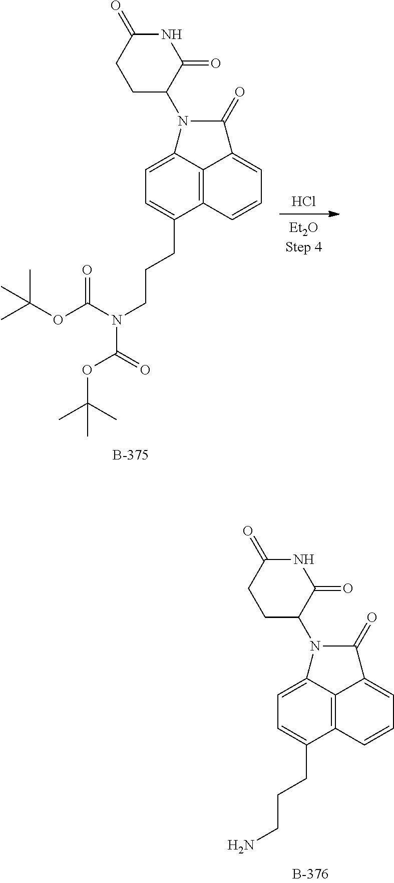 [SMILES: CC(C)(C)OC(=O)N(CCCc1ccc2c3c(cccc13)C(=O)N2C1CCC(=O)NC1=O)C(=O)OC(C)(C)C ; Cl ; NCCCc1ccc2c3c(cccc13)C(=O)N2C1CCC(=O)NC1=O]

### Step 1: 3-(6-bromo-2-oxo-benzo[cd]indol-1-yl)piperidine-2,6-dione (B-372)

3-(6-bromo-2-oxo-benzo[cd]indol-1-yl)piperidine-2,6-dione (B-372, 2.6 g, 5.64 mmol, 56% yield) was synthesized from 6-bromo-1H-benzo[cd]indol-2-one (B-190) and 3-bromopiperidine-2,6-dione (B-139) in a similar fashion to Compound B-3, except using 2.5 eq. B-139 and 5 eq. sodium hydride. The reaction was heated at 60° C. for 16 h. The mixture was cooled to 0° C., quenched with saturated ammonium chloride solution slowly and extracted with ethyl acetate (2×). The combined organic layer was dried over sodium sulfate, filtered and concentrated under reduced pressure. The material was recrystallized from DCM. LCMS (ESI): m/z 358.8 and 360.8 [M+H]⁺.

### Step 2: tert-butyl N-tert-butoxycarbonyl-N—[(E)-3-[1-(2,6-dioxo-3-piperidyl)-2-oxo-benzo[cd]indol-6-yl]allyl]carbamate (B-374)

To a solution of 3-(6-bromo-2-oxo-benzo[cd]indol-1-yl)piperidine-2,6-dione (B-372, 700.0 mg, 1.95 mmol) in DMF (6.0 mL) was added tert-butyl N-allyl-N-tert-butoxycarbonyl-carbamate (B-373, 2.51 g, 9.74 mmol) and N-ethyl-N-isopropyl-propan-2-amine (1.01 g, 7.80 mmol, 1.36 mL). The mixture was purged with argon for 15 min followed by the addition of tris-o-tolylphosphane (296.60 mg, 974.46 μmol) and palladium(II)acetate (109.39 mg, 487.23 μmol). The reaction mixture was heated in a sealed tube at 120° C. for 16 h. The reaction mixture was filtered through Celite, washing with ethyl acetate. The filtrate was washed with water and brine. The combined organic layer was concentrated and the residue purified by column chromatography using 30% Ethyl acetate in hexane to afford tert-butyl N-tert-butoxycarbonyl-N—[(E)-3-[1-(2,6-dioxo-3-piperidyl)-2-oxo-benzo[cd]indol-6-yl]allyl]carbamate (B-374, 900.0 mg, 1.45 mmol, 74% yield). LCMS (ES+): m/z 536.4 [M+H]⁺.

### Step 3: ter-butyl N-tert-butoxycarbonyl-N-[3-[1-(2,6-dioxo-3-piperidyl)-2-oxo-benzo[cd]indol-6-yl]propyl]carbamate (B-375)

tert-butyl N-tert-butoxycarbonyl-N-[3-[1-(2,6-dioxo-3-piperidyl)-2-oxo-benzo[cd]indol-6-yl]propyl]carbamate (B-375, 50 mg, 73.47 μmol, 49% yield) was synthesized from tert-butyl N-tert-butoxycarbonyl-N-[(E)-3-[1-(2,6-dioxo-3-piperidyl)-2-oxo-benzo[cd]indol-6-yl]allyl]carbamate (B-374) in a similar fashion to Compound B-6, except the material was purified by preparatory TLC. LCMS (ES+): m/z 538.2 [M+H]⁺.

### Step 4: 3-[6-(3-aminopropyl)-2-oxo-benzo[cd]indol-1-yl]piperidine-2,6-dione (B-376)

Hydrochloric acid (2 M in diethyl ether) (3.16 mmol, 40.0 mL) was added to a tert-butyl N-tert-butoxycarbonyl-N-[3-[1-(2,6-dioxo-3-piperidyl)-2-oxo-benzo[cd]indol-6-yl]propyl]carbamate (B-375, 1.7 g, 3.16 mmol) at 0° C. After 5 h at room temperature, the solvent was evaporated, and the residue triturated with ether to afford 3-[6-(3-aminopropyl)-2-oxo-benzo[cd]indol-1-yl]piperidine-2,6-dione (B-376, 1.11 g, 2.58 mmol, 82% yield, HCl salt) as a yellow solid. LCMS (ES+): m/z 338.2 [M+H]⁺.

### 3-[1-methyl-6-(4-piperidyl) indazol-3-yl]piperidine-2,6-dione (B-383)

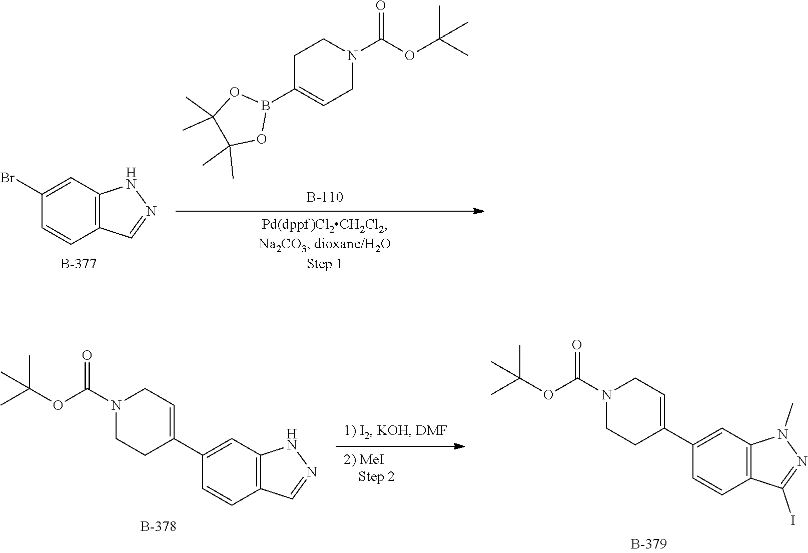 [SMILES: Brc1ccc2cn[nH]c2c1 ; CC(C)(C)OC(=O)N1CC=C(B2OC(C)(C)C(C)(C)O2)CC1 ; CC(C)(C)OC(=O)N1CC=C(c2ccc3cn[nH]c3c2)CC1 ; Cn1nc(I)c2ccc(C3=CCN(C(=O)OC(C)(C)C)CC3)cc21]

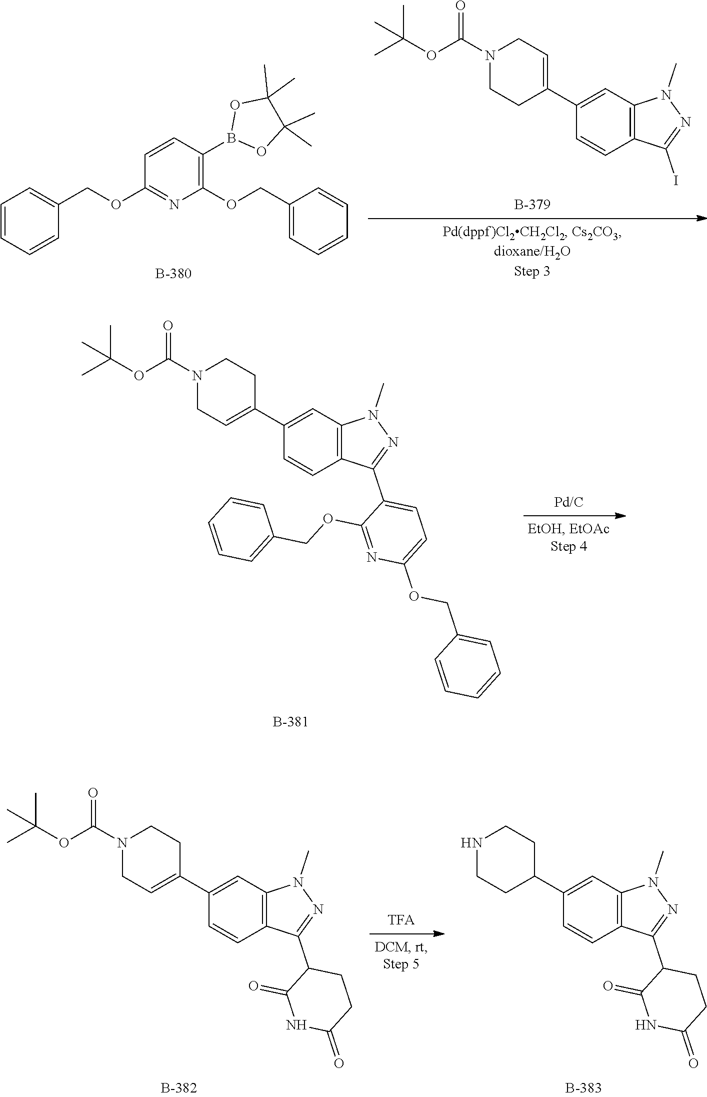 [SMILES: CC1(C)OB(c2ccc(OCc3ccccc3)nc2OCc2ccccc2)OC1(C)C ; Cn1nc(I)c2ccc(C3=CCN(C(=O)OC(C)(C)C)CC3)cc21 ; Cn1nc(-c2ccc(OCc3ccccc3)nc2OCc2ccccc2)c2ccc(C3=CCN(C(=O)OC(C)(C)C)CC3)cc21 ; Cn1nc(C2CCC(=O)NC2=O)c2ccc(C3=CCN(C(=O)OC(C)(C)C)CC3)cc21 ; Cn1nc(C2CCC(=O)NC2=O)c2ccc(C3CCNCC3)cc21]

### Step 1: tert-butyl 4-(1H-indazol-6-yl)-3,6-dihydropyridine-1(2H)-carboxylate (B-378)

A mixture of 6-bromo-1H-indazole (B-377, 57.0 g, 289 mmol), tert-butyl 4-(4,4,5,5-tetramethyl-1,3,2-dioxaborolan-2-yl)-3,6-dihydropyridine-1(2H)-carboxylate (B-110, 134 g, 433 mmol), Pd(dppf)Cl₂·CH₂Cl₂ (12.0 g, 14.6 mmol) and Na₂CO₃ (100 g, 943 mmol) in dioxane (480 mL) and water (120 mL) was stirred at 105° C. for 12 h. The mixture was filtered through Celite and washed with ethyl acetate (500 mL). The filtrate was washed with brine (3×150 mL), dried over sodium sulfate, filtered and concentrated under reduced pressure. The residue was purified by silica gel chromatography (0˜30% ethyl acetate/petroleum ether) to afford tert-butyl 4-(1H-indazol-6-yl)-3,6-dihydropyridine-1(2H)-carboxylate (B-378, 80.0 g, 239 mmol, 83% yield) as yellow oil. LCMS (ES+): m/z 300.1 [M+H]⁺.

### Step 2: tert-butyl 4-(3-iodo-1-methyl-1H-indazol-6-yl)-3,6-dihydropyridine-1(2H)-carboxylate (B-379)

To a solution of tert-butyl 4-(1H-indazol-6-yl)-3,6-dihydropyridine-1(2H)-carboxylate (B-378, 75.0 g, 224 mmol) in DMF (700 mL) was added KOH (37.7 g, 672 mmol) and I₂ (85.3 g, 336 mmol, 67.7 mL). The mixture was stirred at 25° C. for 12 h and was cooled to 0° C. Mel (44.6 g, 314 mmol, 19.6 mL) was then added. The resulting mixture was stirred at 25° C. for 1 h. The mixture was poured into water (1500 mL) and extracted with ethyl acetate (3×500 mL). The combined organic phase was washed with brine (3×500 mL) and dried over sodium sulfate, filtered and concentrated under vacuum to give a residue, which was purified by silica gel chromatography (0˜8% ethyl acetate/petroleum ether) to obtain tert-butyl 4-(3-iodo-1-methyl-1H-indazol-6-yl)-3,6-dihydropyridine-1(2H)-carboxylate (B-379, 23.0 g, 52.3 mmol, 23% yield) as a yellow oil. LCMS (ES+): m/z 440.1 [M+H]⁺.

### Step 3: tert-butyl 4-(3-(2,6-bis(benzyloxy)pyridin-3-yl)-1-methyl-1H-indazol-6-yl)-3,6-dihydropyridine-1(2H)-carboxylate (B-381)

To a solution of 2,6-bis(benzyloxy)-3-(4,4,5,5˜tetramethyl-1,3,2-dioxaborolan-2-yl)pyridine (B-380, 20.0 g, 45.53 mmol), tert-butyl 4-(1H-indazol-6-yl)-3,6-dihydropyridine-1(2H)-carboxylate (B-379, 26.6 g, 63.7 mmol) and Cs₂CO₃ (44.5 g, 136 mmol) in dioxane (200 mL) and water (40 mL) was added Pd(dppf)Cl₂·CH₂Cl₂ (3.72 g, 4.55 mmol, 0.10 eq). The reaction mixture was stirred at 100° C. for 2 h. The reaction mixture was filtered through Celite, and the filtrate was washed with brine (3×60 mL), dried over sodium sulfate, filtered and concentrated. The residue was purified by silica gel chromatography (0˜100% ethyl acetate in petroleum ether) to obtain tert-butyl 4-(3-(2,6-bis(benzyloxy)pyridin-3-yl)-1-methyl-1H-indazol-6-yl)-3,6-dihydropyridine-1(2H)-carboxylate (B-381, 20.0 g, 73% yield) as yellow oil. LCMS (ES+): m/z 603.3 [M+H]⁺.

### Step 4: tert-butyl 4-(3-(2,6-dioxopiperidin-3-yl)-1-methyl-1H-indazol-6-yl)piperidine-1-carboxylate (B-382)

To a solution of tert-butyl 4-(3-(2,6-bis(benzyloxy)pyridin-3-yl)-1-methyl-1H-indazol-6-yl)-3,6-dihydropyridine-1(2H)-carboxylate (B-381, 18.0 g, 29.8 mmol, 1.00 eq) in EtOH (270 mL) and Ethyl acetate (270 mL) was added Pd/C (4.00 g, 10% purity) under a N₂ atmosphere. The suspension was degassed and purged with H₂ 3 times. The mixture was stirred under H₂ (15 psi) at 30° C. for 24 h. The reaction mixture was filtered through Celite and the filtrate was concentrated. The residue was purified by silica gel chromatography (ethyl acetate/petroleum ether) to afford tert-butyl 4-(3-(2,6-dioxopiperidin-3-yl)-1-methyl-1H-indazol-6-yl)piperidine-1-carboxylate (B-382, 5.3 g, 41% yield) as a white solid. LCMS (ES+): m/z 427.2 [M+H]⁺.

### Step 5: 3-[1-methyl-6-(4-piperidyl) indazol-3-yl]piperidine-2,6-dione (B-383)

3-[1-methyl-6-(4-piperidyl) indazol-3-yl]piperidine-2,6-dione (B-383, 500 mg, 1.12 mmol, 95% yield, TFA salt) was synthesized from tert-butyl 4-[3-(2,6-dioxo-3-piperidyl)-1-methyl-indazol-6-yl]piperidine-1-carboxylate (B-382) in a similar fashion to Compound A-62, except using 5 eq. TFA. LCMS (ES+): m/z 327.2 [M+H]⁺.

### 3-[3-(2,6-dioxo-3-piperidyl)-1-methyl-indazol-6-yl]propionic acid (B-390)

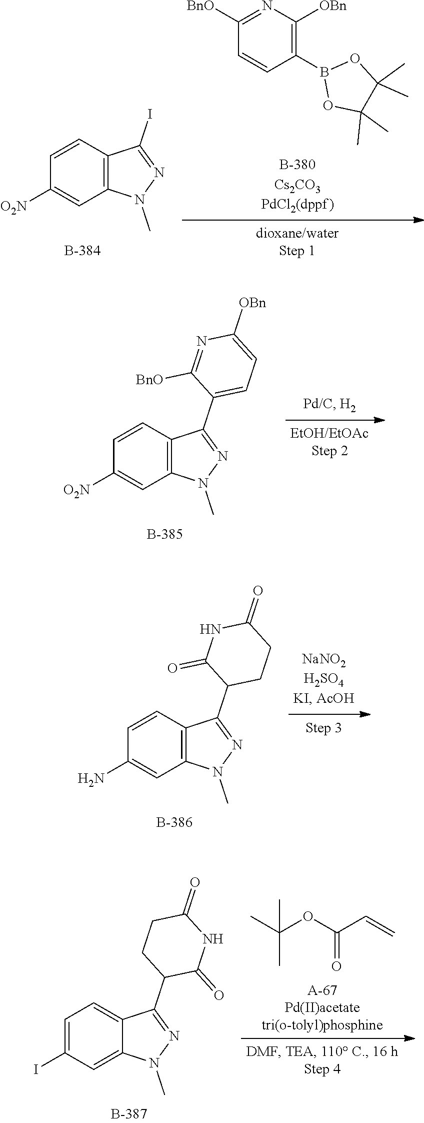

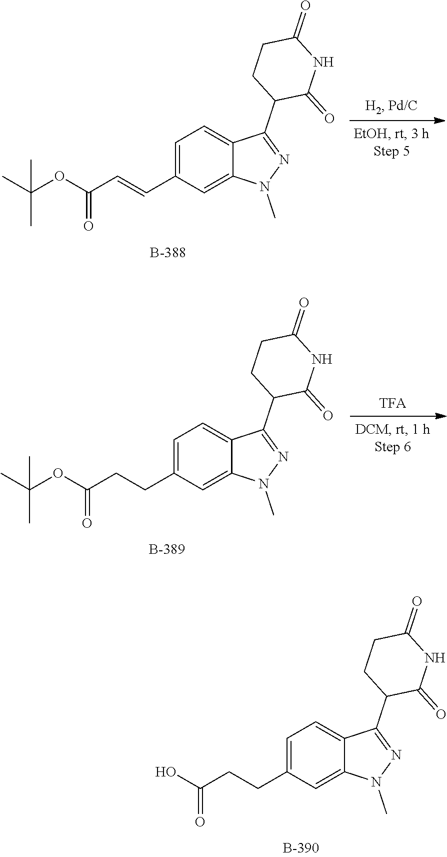 [SMILES: Cn1nc(C2CCC(=O)NC2=O)c2ccc(/C=C/C(=O)OC(C)(C)C)cc21 ; "H2, Pd/C" ; Cn1nc(C2CCC(=O)NC2=O)c2ccc(CCC(=O)OC(C)(C)C)cc21 ; Cn1nc(C2CCC(=O)NC2=O)c2ccc(CCC(=O)O)cc21 ; "TFA"]

### Step 1: 3-(2,6-dibenzyloxy-3-pyridyl)-1-methyl-6-nitro-indazole (B-385)

In a scaled tube, a stirred solution of 3-iodo-1-methyl-6-nitro-indazole (B-384, 2.0 g, 6.60 mmol) and 2,6-dibenzyloxy-3-(4,4,5,5-tetramethyl-1,3,2-dioxaborolan-2-yl)pyridine (B-380, 3.03 g, 7.26 mmol) was purged with argon, before adding cesium carbonate (6.45 g, 19.80 mmol) and [1,1′-Bis(diphenylphosphino)ferrocene]dichloropalladium(II), complex with dichloromethane (538.93 mg, 659.94 μmol). The reaction mixture was heated at 100° C. for 16 h. The reaction mixture was cooled to room temperature, filtered through Celite and the aqueous part was separated. The solvent was removed and the residue was purified by silica gel column chromatography to afford 3-(2,6-dibenzyloxy-3-pyridyl)-1-methyl-6-nitro-indazole (B-385, 1.6 g, 3.43 mmol, 52% yield) as yellowish solid. LCMS (ES+): m/z 467 [M+H]⁺.

### Step 2: 3-(6-amino-1-methyl-indazol-3-yl)piperidine-2,6-dione (B-386)

To a stirred solution of 3-(2,6-dibenzyloxy-3-pyridyl)-1-methyl-6-nitro-indazole (B-385, 5 g, 10.72 mmol) in 1:1 mixture of EtOH (125 mL) and Ethyl acetate (125 mL) was added Palladium (10% carbon, 50% wet) (5 g), and the mixture was stirred under an atmosphere of hydrogen (bladder). The reaction mixture was filtered through Celite, concentrated and the residue was purified by silica gel column chromatography to afford 3-(6-amino-1-methyl-indazol-3-yl)piperidine-2,6-dione (B-386, 1.05 g, 4.07 mmol, 38% yield) as reddish-brown solid. LCMS (ES+): m/z 259.1 [M+H]⁺.

### Step 3: 3-(6-iodo-1-methyl-indazol-3-yl)piperidine-2,6-dione (B-387)

To 3-(6-amino-1-methyl-indazol-3-yl)piperidine-2,6-dione (B-386, 450 mg, 1.74 mmol) was added Acetic acid (1.8 mL) and sulfuric acid (342 mg, 3.49 mmol) at −10° C. Subsequently, NaNO₂ (175 mg, 2.54 mmol) and KI (840 mg, 5.06 mmol) were added slowly. The reaction was stirred for 1.5 h at −10° C. Water was added and extracted with Ethyl acetate. The organic layer was washed with a sodium thiosulfate solution and concentrated under reduced pressure. The residue was purified by silica gel column chromatography to afford 3-(6-iodo-1-methyl-indazol-3-yl)piperidine-2,6-dione (B-387, 210 mg, 522.90 μmol, 30% yield) as light-yellow solid. LCMS (ES+): m/z 370.0 [M+H]⁺. ¹H NMR (400 MHz, DMSO-d6) δ 10.89 (s, 1H), 8.11 (s, 1H), 7.52 (d, J=12 Hz, 1H), 7.38 (d, J=12 Hz, 1H), 4.35 (m, 1H), 3.97 (s, 3H), 2.60 (m, 2H), 2.32 (m, 1H), 2.13 (m, 1H).

### Step 4: tert-butyl (E)-3-[3-(2,6-dioxo-3-piperidyl)-1-methyl-indazol-6-yl]prop-2-enoate (B-388)

Into a 25 mL single neck round bottom flask containing a well-stirred solution of 3-(6-iodo-1-methyl-indazol-3-yl)piperidine-2,6-dione (B-387, 0.200 g, 541.78 μmol) and tert-butyl prop-2-enoate (A-67, 138.88 mg, 1.08 mmol, 157.28 μL) in anhydrous DMF (5 mL) were added triethylamine (54.82 mg, 541.78 μmol, 75.51 μL) and palladium(II)acetate (12.16 mg, 54.18 μmol) followed by tri (o-tolyl)phosphine (164.90 mg, 541.78 μmol) under a nitrogen atmosphere. The reaction mixture was heated at 100° C. for 16 h. The solvent was removed under reduced pressure and the residue purified by reverse phase prep HPLC (Column: XBridge C18 150 mm; 0.1% TFA in water/MeCN) to afford tert-butyl (E)-3-[3-(2,6-dioxo-3-piperidyl)-1-methyl-indazol-6-yl]prop-2-enoate (B-388, 0.150 g, 400 μmol, 74% yield) as a white solid. LCMS (ES+): m/z 370.0 [M+H]⁺.

### Step 5: tert-butyl 3-[3-(2,6-dioxo-3-piperidyl)-1-methyl-indazol-6-yl]propionate (B-389)

Into a 50 mL single neck round bottom flask containing a well-stirred solution of tert-butyl (E)-3-[3-(2,6-dioxo-3-piperidyl)-1-methyl-indazol-6-yl]prop-2-enoate (B-388, 0.150 g, 406.05 μmol) in ethanol (15 mL) was added Pd/C (10% dry) (0.05 g, 406.05 μmol) under a nitrogen atmosphere. The nitrogen was evacuated and back filled with hydrogen using a bladder. The resulting mixture was stirred under a hydrogen atmosphere for 3 h. The reaction mixture was filtered through Celite, washed with ethanol (50 mL) and the filtrate concentrated to afford tert-butyl 3-[3-(2,6-dioxo-3-piperidyl)-1-methyl-indazol-6-yl]propionate (B-389, 0.120 g, 313.83 μmol, 77% yield) as an off white solid. The material was used in the next step without further purification. LCMS (ES+): m/z 371.9 [M+H]⁺.

### Step 6: 3-[3-(2,6-dioxo-3-piperidyl)-1-methyl-indazol-6-yl]propionic acid (B-390)

3-[3-(2,6-dioxo-3-piperidyl)-1-methyl-indazol-6-yl]propionic acid (B-390, 0.100 g, 301.67 μmol, 100% yield) was synthesized from tert-butyl 3-[3-(2,6-dioxo-3-piperidyl)-1-methyl-indazol-6-yl]propanoate (B-389) in a similar fashion to Compound A-26, except using 40 eq. TFA. The residue was triturated with diethyl ether. LCMS (ES−): m/z 314.7 [M−H]⁻.

### tert-butyl(S)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144a) and tert-butyl (R)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144b)

Configurations are arbitrarily assigned.

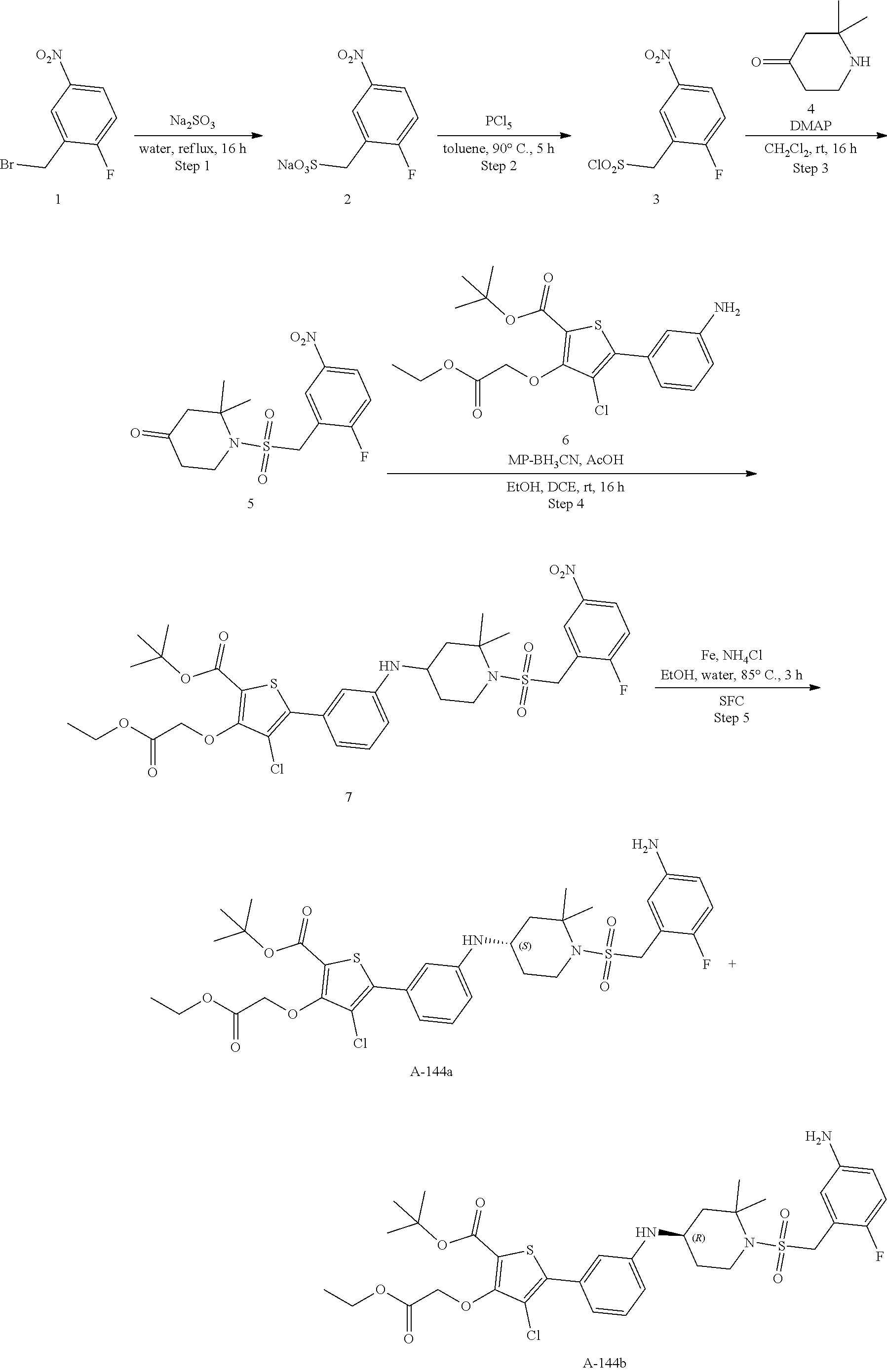

### Step 1: sodium (2-fluoro-5-nitrophenyl)methanesulfonate (2)

sodium (2-fluoro-5-nitrophenyl)methanesulfonate (2, 5.0 g, 17.86 mmol, 84% yield) was synthesized from 2-(bromomethyl)-1-fluoro-4-nitro-benzene (1, 5.0 g, 21.37 mmol) in a similar fashion to Compound B-154, except using 1 eq. sodium sulphite. The material was used in the next step without further characterization.

### Step 2: (2-fluoro-5-nitrophenyl)methane sulfonyl chloride (3)

(2-fluoro-5-nitro-phenyl)methane sulfonyl chloride (3, 9.0 g, 25.37 mmol, 82% yield) was synthesized from (2-fluoro-5-nitro-phenyl)methylsulfonyloxy sodium (2, 8.0 g, 31.11 mmol) in a similar fashion to Compound B-155. The material was used in the next step without further purification.

### Step 3: 1-((2-fluoro-5-nitrobenzyl)sulfonyl)-2,2-dimethylpiperidin-4-one (5)

Into a 250 mL single neck round bottom flask containing a well-stirred solution of 2,2-dimethylpiperidin-4-one (4, 1.45 g, 8.86 mmol, HCl salt) in dry DCM (100 mL) were added DMAP (3.25 g, 26.59 mmol, 1.33 mL) and (2-fluoro-5-nitro-phenyl)methane sulfonyl chloride (4, 9.42 g, 26.59 mmol) at 0° C. under nitrogen atmosphere. The reaction was stirred for 16 h at rt. The solvent was removed and the residue purified via flash column chromatography using the silica gel (230-400 mesh silica gel, 50% EtOAc in pet ether) to afford 1-[(2-fluoro-5-nitro-phenyl) methyl sulfonyl]-2,2-dimethyl-piperidin-4-one (5, 2.6 g, 5.04 mmol, 57% yield) as an off white solid.

LCMS (ES−): m/z 343.0 [M−H]⁻.

### Step 4: tert-butyl 4-chloro-3-(2-ethoxy-2-oxo-ethoxy)-5-[3-[[1-[(2-fluoro-5-nitro-phenylmethylsulfonyl]-2,2-dimethyl-4-piperidyl]amino]phenyl]thiophene-2-carboxylate (7)

tert-butyl 4-chloro-3-(2-ethoxy-2-oxo-ethoxy)-5-[3-[[1-[(2-fluoro-5-nitro-phenyl)methylsulfonyl]-2,2-dimethyl-4-piperidyl]amino]phenyl]thiophene-2-carboxylate (7, 3.3 g, 3.11 mmol, 59% yield) was synthesized from 1-[(2-fluoro-5-nitro-phenyl)methylsulfonyl]-2,2-dimethyl-piperidin-4-one (5, 2.6 g, 5.29 mmol) and tert-butyl 5-(3-aminophenyl)-4-chloro-3-(2-ethoxy-2-oxo-ethoxy)thiophene-2-carboxylate (6, 2.18 g, 5.29 mmol) in a similar fashion to Compound A-74a/b, except using a 1:1 mixture of EtOH/DCE, 3 eq. MP-CNBH₃ and 10 eq. AcOH.

LCMS (ES+): m/z 740.0 [M+H]⁺.

### Step 5: tert-butyl(S)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144a) and tert-butyl (R)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144b)

tert-butyl-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144, 400 mg, 91% yield) was synthesized from tert-butyl 4-chloro-3-(2-ethoxy-2-oxo-ethoxy)-5-[3-[[1-[(2-fluoro-5-nitro-phenyl)methylsulfonyl]-2,2-dimethyl-4-piperidyl]amino]phenyl]thiophene-2-carboxylate (7, 500 mg, 617.35 μmol) in a similar fashion to Compound B-99.

Chiral Separation: The enantiomers were separated by chiral SFC: Method details: Column Name: Lux A1; Co-Solvent: 40% and Co-Solvent Name: 0.5% Isopropyl Amine in MeOH; Outlet Pressure: 100 bar; Temperature: 35° C.

After the separation first eluted isomer tert-butyl (S)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144a, 110 mg, 154.58 μmol, 25% yield)(RT 2.66, optical purity 99.81%), was isolated as an off-white solid.

LCMS (ES+): m/z 710.0 [M+H]⁺.

And the second eluted isomer tert-butyl (R)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144b, 110 mg, 153.83 μmol, 25% yield) (RT 3.84, optical purity 99.33%), was isolated as an off-white solid.

LCMS (ES+): m/z 710.0 [M+H]⁺.

### 5-[3-[[(4S)-1-[(5-amino-2-fluoro-phenyl) methyl sulfonyl]-2,2-dimethyl-4-piperidyl]amino]phenyl]-3-(carboxymethoxy)-4-chloro-thiophene-2-carboxylic acid (A-145a)

Configurations are arbitrarily assigned.

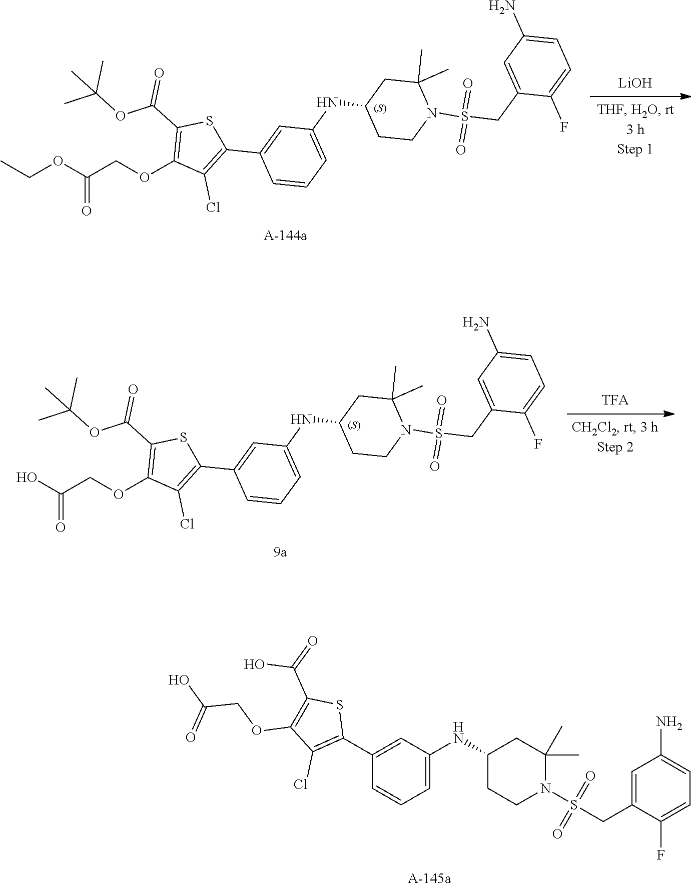

### Step 1: 2-[[5-[3-[[(4S)-1-[(5-amino-2-fluoro-phenyl)methylsulfonyl]-2,2-dimethyl-4-piperidyl]amino]phenyl]-2-tert-butoxycarbonyl-4-chloro-3-thienyl]oxy]acetic acid (9a)

2-[[5-[3-[[(4S)-1-[(5-amino-2-fluoro-phenyl)methylsulfonyl]-2,2-dimethyl-4-piperidyl]amino]phenyl]-2-tert-butoxycarbonyl-4-chloro-3-thienyl]oxy]acetic acid (9a, 450 mg, 610.40 μmol, 88% yield) was synthesized from tert-butyl(S)-5-(3-((1-((5-amino-2-fluorobenzyl)sulfonyl)-2,2-dimethylpiperidin-4-yl)amino)phenyl)-4-chloro-3-(2-ethoxy-2-oxoethoxy)thiophene-2-carboxylate (A-144a, 500 mg, 696.91 μmol) in a similar fashion to Compound A-9, except using 2 eq. lithium hydroxide monohydrate.

LCMS (ES−): m/z 680.0 [M−H]⁻.

### Step 2: 5-[3-[[(4S)-1-[(5-amino-2-fluoro-phenyl) methyl sulfonyl]-2,2-dimethyl-4-piperidyl]amino]phenyl]-3-(carboxymethoxy)-4-chloro-thiophene-2-carboxylic acid (A-145a)

5-[3-[[(4S)-1-[(5-amino-2-fluoro-phenyl)methylsulfonyl]-2,2-dimethyl-4-piperidyl]amino]phenyl]-3-(carboxymethoxy)-4-chloro-thiophene-2-carboxylic acid (A-145a, 400 mg, 591.60 μmol, 88% yield, TFA salt) was synthesized from 2-[5-[3-[[(4S)-1-[(5-amino-2-fluoro-phenyl)methylsulfonyl]-2,2-dimethyl-4-piperidyl]amino]phenyl]-2-tert-butoxycarbonyl-4-chloro-3-thienyl]oxy]acetic acid (9a, 450 mg, 609.48 μmol) in a similar fashion to Compound A-26, except using 5 eq. TFA and triturating the product with diethyl ether. LCMS (ES+): m/z 626 [M+H]⁺.

### 5-[3-[[(4R)-1-[(5-amino-2-fluoro-phenyl) methyl sulfonyl]-2,2-dimethyl-4-piperidyl]amino]phenyl]-3-(carboxymethoxy)-4-chloro-thiophene-2-carboxylic acid A-145b)

Configurations are arbitrarily assigned.

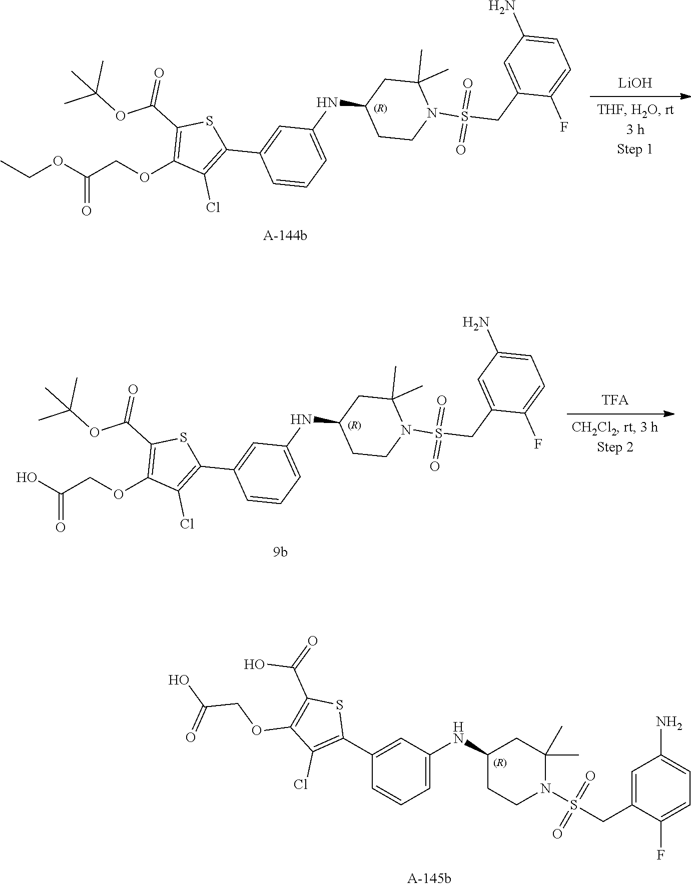

### Step 1: 2-[5-[3-[[(4R)-1-[(5-amino-2-fluoro-phenyl) methyl sulfonyl]-2,2-dimethyl-4-piperidyl]amino]phenyl]-2-tert-butoxycarbonyl-4-chloro-3-thienyl]oxy]acetic acid (9b)

2-[[5-[3-[[(4R)-1-[(5-amino-2-fluoro-phenyl)methylsulfonyl]-2,2-dimethyl-4-piperidyl]amino]phenyl]-2-tert-butoxycarbonyl-4-chloro-3-thienyl]oxy]acetic acid (9b, 460 mg, 553.84 μmol, 79% yield) was synthesized from tert-butyl 5-[3-[[(4R)-1-[(5-amino-2-fluoro-phenyl)methylsulfonyl]-2,2-dimethyl-4-piperidyl]amino]phenyl]-4-chloro-3-(2-ethoxy-2-oxo-ethoxy)thiophene-2-carboxylate (A-144b, 500 mg, 696.91 μmol) in a similar fashion to Compound A-9, except using 2 eq. lithium hydroxide monohydrate. LCMS (ES−): m/z 680 [M−H]⁻.

### Step 2: 5-[3-[[(4R)-1-[(5-amino-2-fluoro-phenyl) methyl sulfonyl]-2,2-dimethyl-4-piperidyl] amino] phenyl]-3-(carboxymethoxy)-4-chloro-thiophene-2-carboxylic acid (A-145b)

5-[3-[[(4R)-1-[(5-amino-2-fluoro-phenyl)methylsulfonyl]-2,2-dimethyl-4-piperidyl]amino]phenyl]-3-(carboxymethoxy)-4-chloro-thiophene-2-carboxylic acid (A-145b, 400 mg, 581.43 μmol, 99% yield, TFA salt) was synthesized from 2-[[5-[3-[[(4R)-1-[(5-amino-2-fluoro-phenyl)methylsulfonyl]-2,2-dimethyl-4-piperidyl]amino]phenyl]-2-tert-butoxycarbonyl-4-chloro-3-thienyl]oxy]acetic acid (9b, 490 mg, 589.96 μmol) in a similar fashion to Compound A-26, except using 5 eq. TFA and triturating the product with diethyl ether. LCMS (ES+): m/z 626 [M+H]⁺.

### (S)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate and (R)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (A-146A, A-146B)

Configurations are arbitrarily assigned.

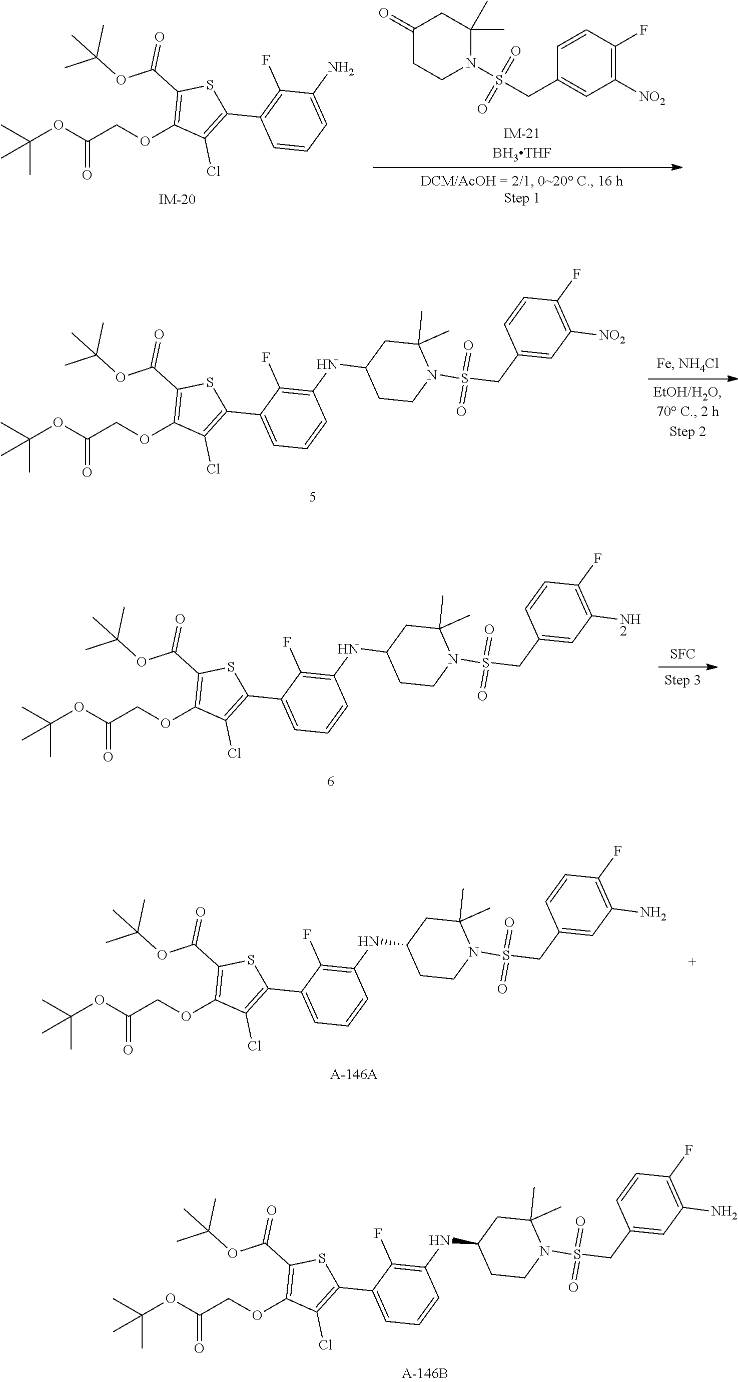

### Step 1: tert-butyl 3-(2-(tert-butoxy)-2-oxoethoxy)-4-chloro-5-(2-fluoro-3-((1-((4-fluoro-3-nitrobenzyl)sulfonyl)-2,2-dimethylpiperidin-4-yl)amino)phenyl)thiophene-2-carboxylate (5)

To a mixture of tert-butyl 5-(3-amino-2-fluorophenyl)-3-(2-(tert-butoxy)-2-oxoethoxy)-4-chlorothiophene-2-carboxylate (IM-20, 3 g, 6.55 mmol), 1-((4-fluoro-3-nitrobenzyl)sulfonyl)-2,2-dimethylpiperidin-4-one (IM-21, 2.37 g, 6.88 mmol) in DCM (30 mL) and AcOH (15 mL) was added BH₃·THF (1 M, 19.65 mL) at 0° C. under N₂, the mixture was stirred at 20° C. for 16 h. MeOH (30 mL) was added to the mixture and the mixture was stirred at rt for 1 h. The mixture concentrated in vacuum. The residue was diluted with EtOAc (100 mL) and H₂O (300 mL). The mixture was extracted with EtOAc (100 mL). The organic phase was washed with brine (300 mL), dried with anhydrous Na₂SO₄, filtered and concentrated in vacuum. The residue was purified by column chromatography (SiO₂, Petroleum ether/Ethyl acetate=100/1 to 3/1) to afford tert-butyl 3-(2-(tert-butoxy)-2-oxoethoxy)-4-chloro-5-(2-fluoro-3-((1-((4-fluoro-3-nitrobenzyl)sulfonyl)-2,2-dimethylpiperidin-4-yl)amino)phenyl)thiophene-2-carboxylate (5, 2.3 g, 2.93 mmol, 45% yield) as a yellow solid.

LCMS (ESI): m/z 786.4 [M+H]⁺.

### Step 2: tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (6)

tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (6, 1.8 g, 2.38 mmol, 94% yield) was synthesized from tert-butyl 3-(2-(tert-butoxy)-2-oxoethoxy)-4-chloro-5-(2-fluoro-3-((1-((4-fluoro-3-nitrobenzyl)sulfonyl)-2,2-dimethylpiperidin-4-yl)amino)phenyl)thiophene-2-carboxylate (5, 2 g, 2.54 mmol) in a similar fashion to Compound B-99, except using 5 eq. Fe and 5 eq. NH₄Cl. Upon reaction completion, the mixture was filtered and concentrated in vacuum. The residue was diluted with EtOAc (200 mL) and water (500 mL). The mixture was extracted with EtOAc (200 mL). The organic phase was washed with brine (500 mL), dried with anhydrous Na₂SO₄, filtered and concentrated. The material was used in the next step without further purification.

LCMS (ESI): m/z 756.5 [M+H]⁺.

### Step 3: (S)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate, (R)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (A-146A, A-146B)

650 mg of tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate was separated by SFC (Mobile Phase: 45% EtOH (0.1% NH₃·H₂O) in Supercritical CO₂ Flow Rate: 70 g/min Cycle Time: 3.6 min, total time: 40 min Single injection volume: 2.5 ml Back Pressure: 100 bar to keep the CO₂ in Supercritical flow: DAICEL CHIRALPAK AD (250 mm×30 mm, 10 μm) to afford(S)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (A-146A, 310 mg, 409.88 μmol, 48% yield, 100% ee, Peak1) and (R)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (A-146B, 330 mg, 414.51 μmol, 48% yield, 98% ee, Peak2) as white solid.

LCMS (ESI): m/z 778.2 [M+Na]⁺.

### 5-[3-[[(4S)-1-[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147)

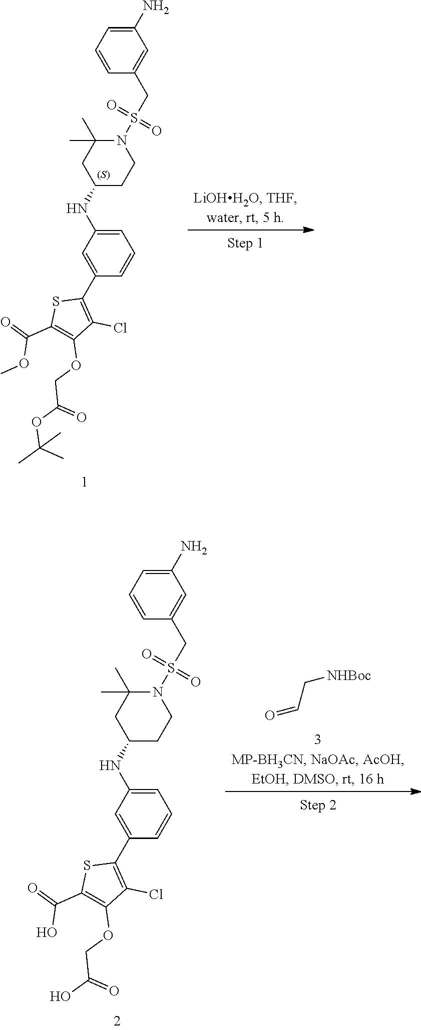

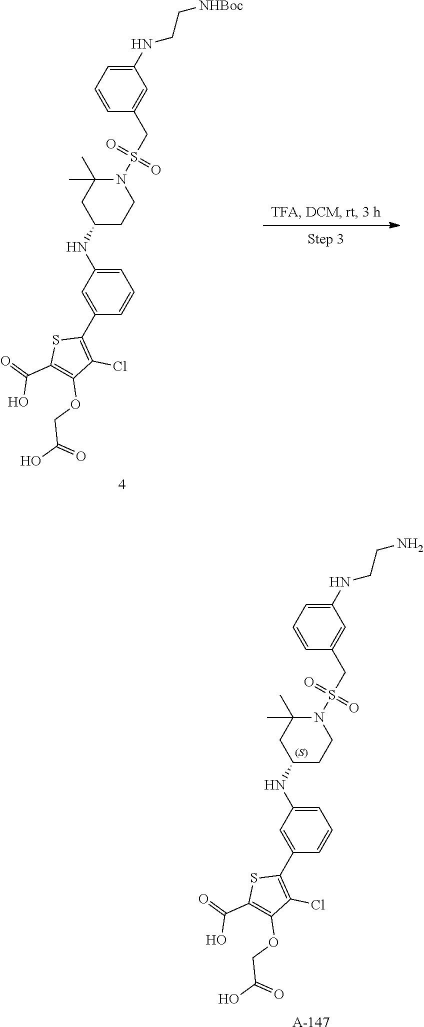

### Step 1: 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (2)

5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (2, 1.6 g, 2.60 mmol, 88% yield) was synthesized from methyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (1, 2 g, 2.95 mmol) in a similar fashion to Compound A-9, except using 8 eq. lithium hydroxide monohydrate. The solvents were removed under reduced pressure to a small volume. The concentrate was acidified with citric acid solution. The solid was filtered, washed with water and dried under reduced pressure.

LCMS (ES+): m/z 608.0 [M+H]⁺.

### Step 2: 5-[3-[[(4S)-1-[[3-[2-(tert-butoxycarbonylamino)ethylamino]phenyl]methylsulfonyl]-2,2-dimethyl-4-piperidyl]amino]phenyl]-3-(carboxymethoxy)-4-chloro-thiophene-2-carboxylic acid (4)

5-[3-[[(4S)-1-[[3-[2-(tert-butoxycarbonylamino)ethylamino]phenyl]methylsulfonyl]-2,2-dimethyl-4-piperidyl]amino]phenyl]-3-(carboxymethoxy)-4-chloro-thiophene-2-carboxylic acid (4, 0.7 g, 739.92 μmol, 29% yield, Formic acid salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (2, 1.6 g, 2.58 mmol) and tert-butyl N-(2-oxoethyl) carbamate (3, 451.48 mg, 2.84 mmol) in a similar fashion to Compound A-74a/b, except using 68 eq. AcOH. The material was purified by reverse phase column chromatography [Purification method: Silicycle C18 column, mobile phase A: 0.1% Formic acid in water; Mobile phase B: MeCN]

LCMS (ES+): m/z 751.2 [M+H]⁺.

### Step 3: 5-[3-[[(4S)-1-[[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147)

5-[3-[[(4S)-1-[[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147, 0.5 g, 607.67 μmol, 89% yield, TFA salt) was synthesized from 5-[3-[[(4S)-1-[3-[2-(tert-butoxycarbonylamino)ethylamino]phenyl]methylsulfonyl]-2,2-dimethyl-4-piperidyl]amino]phenyl]-3-(carboxymethoxy)-4-chloro-thiophene-2-carboxylic acid (4, 0.7 g, 739.92 μmol, Formic acid salt) in a similar fashion to Compound A-62, except using 35 eq. TFA and triturating the product with MTBE.

LCMS (ES+): m/z 651.0 [M+H]⁺.

### (S)-methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (A-148) and (R)-methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (A-149)

Configurations are arbitrarily assigned.

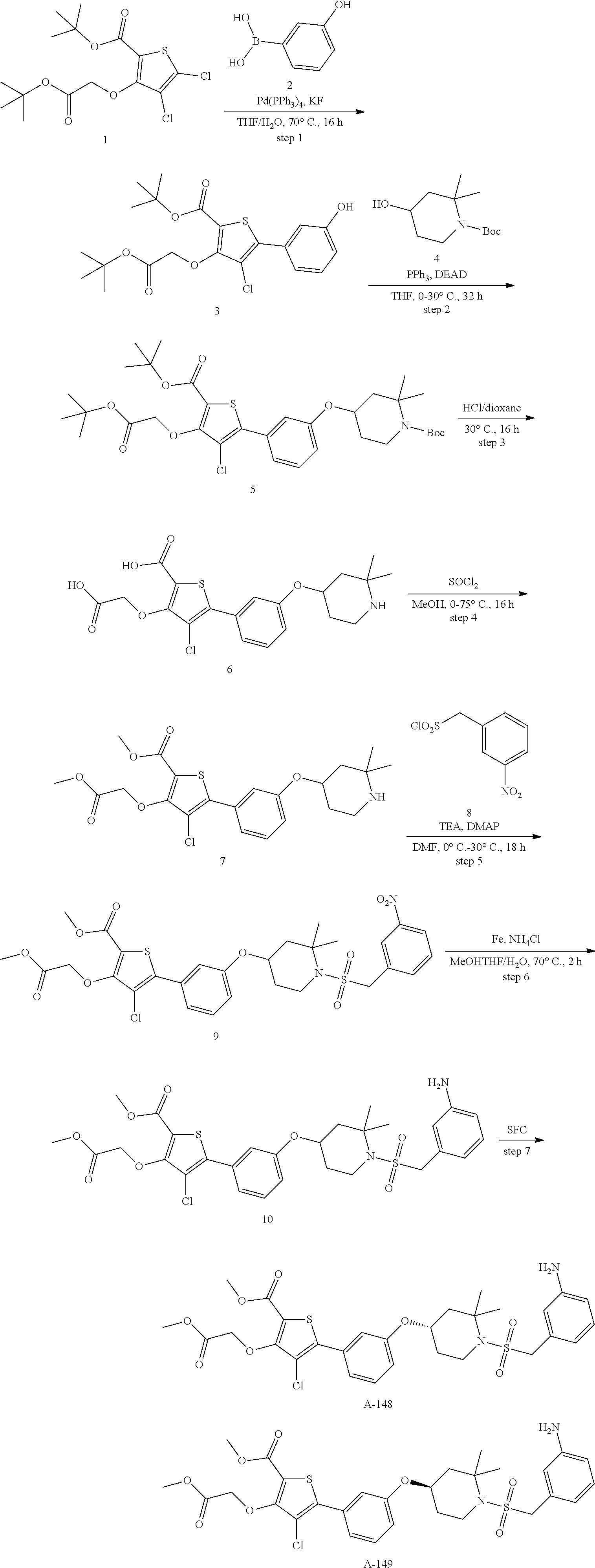

### Step 1: tert-butyl 3-(2-(tert-butoxy)-2-oxoethoxy)-4-chloro-5-(3-hydroxyphenyl)thiophene-2-carboxylate (3)

To a mixture of tert-butyl 3-(2-(tert-butoxy)-2-oxoethoxy)-4,5-dichlorothiophene-2-carboxylate (1, 2 g, 5.22 mmol) and (3-hydroxyphenyl)boronic acid (2, 1.1 g, 7.98 mmol) in THF (16 mL) and Water (4 mL) were added KF (909.50 mg, 15.65 mmol) and Pd(PPh₃)₄ (602.97 mg, 521.80 μmol). The mixture was stirred at 70° C. for 16 h. The mixture was concentrated under reduced pressure to give a residue. The residue was purified by column chromatography (SiO₂, petroleum ether/ethyl acetate=20/1˜5/1; petroleum ether/ethyl acetate=10/1, Rf=0.5) to afford tert-butyl 3-(2-(tert-butoxy)-2-oxoethoxy)-4-chloro-5-(3-hydroxyphenyl)thiophene-2-carboxylate (3, 1 g, 2.22 mmol, 43% yield) as a yellow solid. LCMS (ESI): m/z 463.0 [M+Na]+

¹H NMR (400 MHz, DMSO-d6) δ 9.83 (s, 1H), 7.36-7.28 (m, 1H), 7.13-7.05 (m, 2H), 6.90-6.87 (m, 1H), 4.85 (s, 2H), 1.52 (s, 9H), 1.41 (s, 9H).

### Step 2: tert-butyl 4-(3-(4-(2-(tert-butoxy)-2-oxoethoxy)-5-(tert-butoxycarbonyl)-3-chlorothiophen-2-yl) phenoxy)-2,2-dimethylpiperidine-1-carboxylate (5)

To a solution of tert-butyl 3-(2-(tert-butoxy)-2-oxoethoxy)-4-chloro-5-(3-hydroxyphenyl)thiophene-2-carboxylate (3, 2.4 g, 5.44 mmol) and tert-butyl 4-hydroxy-2,2-dimethylpiperidine-1-carboxylate (4, 1.50 g, 6.53 mmol) in THF (50 mL) were added triphenylphosphane (2.86 g, 10.89 mmol) and ethyl (NE)-N-ethoxycarbonyliminocarbamate (1.90 g, 10.89 mmol) at 0-10° C. and the mixture was stirred at 30° C. for 16 h. Tert-butyl 4-hydroxy-2,2-dimethylpiperidine-1-carboxylate (4, 1.25 g, 5.44 mmol), triphenylphosphane (2.86 g, 10.89 mmol) and Diethyl azodicarboxylate (1.90 g, 10.89 mmol) were added and the mixture was stirred at 30° C. for 16 h. The reaction mixture was diluted with water (100 mL) and extracted with ethyl acetate (50 mL×2). The combined organic layers were washed with brine (50 mL×2), dried over Na₂SO₄, filtered and concentrated under reduced pressure. The residue was purified by column chromatography (SiO₂, petroleum ether/ethyl acetate=1/0 to 3/1; petroleum ether/ethyl acetate=5/1, Rf=0.6) to afford tert-butyl 4-(3-(4-(2-(tert-butoxy)-2-oxoethoxy)-5-(tert-butoxycarbonyl)-3-chlorothiophen-2-yl) phenoxy)-2,2-dimethylpiperidine-1-carboxylate (5, 2 g, 2.91 mmol, 54% yield) as colorless oil.

LCMS (ESI): m/z 552.3 [M−99]+,

¹H NMR (400 MHz, CHLOROFORM-d) δ 7.37-7.31 (m, 1H), 7.24-7.18 (m, 2H), 6.92 (dd, J=1.6, 8.0 Hz, 1H), 4.79 (s, 2H), 4.63-4.54 (m, 1H), 3.75-3.70 (m, 1H), 3.46 (ddd, J=4.1, 8.1, 13.8 Hz, 1H), 2.25-2.14 (m, 1H), 2.00-1.84 (m, 2H), 1.83-1.73 (m, 1H), 1.60 (s, 3H), 1.57 (s, 9H), 1.53 (s, 3H), 1.49 (s, 9H), 1.47 (s, 9H), 1.42 (s, 3H).

### Step 3: 3-(carboxymethoxy)-4-chloro-5-[3-[(2,2-dimethyl-4-piperidyl)oxy]phenyl]thiophene-2-carboxylic acid (6)

A solution of tert-butyl 4-(3-(4-(2-(tert-butoxy)-2-oxoethoxy)-5-(tert-butoxycarbonyl)-3-chlorothiophen-2-yl) phenoxy)-2,2-dimethylpiperidine-1-carboxy late (5, 1.7 g, 2.61 mmol) in HCl/dioxane (4 M, 30 mL) was stirred at 30° C. for 16 b. The mixture was concentrated under reduced pressure to afford 3-(carboxymethoxy)-4-chloro-5-[3-[(2,2-dimethyl-4-piperidyl)oxy]phenyl]thiophene-2-carboxylic acid (6, 1.2 g, 2.52 mmol, 97% yield, HCl salt) as a yellow solid.

LCMS (ESI): m/z 440.2 [M+H]⁺.

### Step 4: methyl 4-chloro-5-(3-((2,2-dimethylpiperidin-4-yl)oxy)phenyl)-3-(2-methoxy-2-oxoethoxy)thiophene-2-carboxylate (7)

To a solution of 3-(carboxymethoxy)-4-chloro-5-[3-[(2,2-dimethyl-4-piperidyl)oxy]phenyl]thiophene-2-carboxylic acid (6, 1.2 g, 2.52 mmol, HCl salt) in MeOH (30 mL) was added thionyl chloride (4.92 g, 41.35 mmol, 3 mL) at 0-30° C. The mixture was stirred at 75° C. for 16 h. The reaction mixture was concentrated under reduced pressure to afford methyl 4-chloro-5-(3-((2,2-dimethylpiperidin-4-yl)oxy)phenyl)-3-(2-methoxy-2-oxoethoxy)thiophene-2-carboxylate (7, 1.2 g, 2.38 mmol, 94% yield) as yellow oil. The material was used in the next step without purification.

LCMS (ESI): m/z 468.0 [M+H]⁺.

### Step 5: methyl 4-chloro-5-(3-((2,2-dimethyl-1-((3-nitrobenzyl)sulfonyl)piperidin-4-yl)oxy)phenyl)-3-(2-methoxy-2-oxoethoxy)thiophene-2-carboxylate (9)

To a solution of methyl 4-chloro-5-(3-((2,2-dimethylpiperidin-4-yl)oxy)phenyl)-3-(2-methoxy-2-oxoethoxy)thiophene-2-carboxylate (7, 1.2 g, 2.38 mmol, HCl salt), TEA (1.20 g, 11.89 mmol, 1.66 mL) and DMAP (2.91 g, 23.79 mmol) in DMF (30 mL) was added (3-nitrophenyl)methanesulfonyl chloride (8, 1.20 g, 5.09 mmol) at 0-10° C. The mixture was stirred at 30° C. for 16 h. The reaction mixture was diluted with water (200 mL) and extracted with ethyl acetate (100 mL×2). The combined organic layers were washed with brine (100 mL×2), dried over Na₂SO₄, filtered and concentrated under reduced pressure. The residue was purified by column chromatography (SiO₂, petroleum ether/ethyl acetate=1/0 to 1/1 and then DCM/MeOH=5/1; petroleum ether/ethyl acetate=1/1, Rf=0.3) to afford methyl 4-chloro-5-(3-((2,2-dimethyl-1-((3-nitrobenzyl)sulfonyl)piperidin-4-yl)oxy)phenyl)-3-(2-methoxy-2-oxoethoxy)thiophene-2-carboxy late (9, 450 mg, 647.53 μmol, 27% yield) as yellow oil.

LCMS (ESI): m/z 667.0 [M+H]⁺.

¹H NMR (400 MHz, CHLOROFORM-d) δ 8.34-8.23 (m, 2H), 7.78 (d, J=7.7 Hz, 1H), 7.65-7.57 (m, 1H), 7.39-7.31 (m, 1H), 7.24 (d, J=8.0 Hz, 1H), 7.18 (t, J=1.6 Hz, 1H), 6.92 (dd, J=2.0, 8.0 Hz, 1H), 4.94 (s, 2H), 4.49 (tt, J=4.0, 8.4 Hz, 1H), 4.40-4.27 (m, 2H), 3.88 (s, 3H), 3.83 (s, 3H), 3.54-3.43 (m, 1H), 3.24-3.18 (m, 1H), 1.99-1.95 (m, 1H), 1.93-1.85 (m, 1H), 1.80-1.77 (m, 1H), 1.64 (s, 3H), 1.52 (s, 3H), 1.49-1.37 (m, 1H).

### Step 6: methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (10)

Methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (10, 500 mg, 753.34 μmol, 87% yield) was synthesized from methyl 4-chloro-5-(3-((2,2-dimethyl-1-((3-nitrobenzyl)sulfonyl)piperidin-4-yl)oxy)phenyl)-3-(2-methoxy-2-oxoethoxy)thiophene-2-carboxylate (9, 580 mg, 869.38 μmol) in a similar fashion to Compound B-99, except using 5 eq. Fe and 5 eq. NH₄Cl.

LCMS (ESI): m/z 231.1 [M+H]⁺.

¹H NMR (400 MHz, CHLOROFORM-d) δ 7.39-7.30 (m, 1H), 7.23 (d, J=7.6 Hz, 1H), 7.20-7.12 (m, 2H), 6.91 (dd, J=1.6, 8.0 Hz, 1H), 6.76 (d, J=2.0 Hz, 2H), 6.68 (d, J=8.0 Hz, 1H), 4.94 (s, 2H), 4.44 (td, J=4.4, 8.8 Hz, 1H), 4.23-4.13 (m, 2H), 3.88 (s, 3H), 3.83 (s, 3H), 3.78-3.66 (m, 2H), 3.49-3.39 (m, 1H), 3.23-3.12 (m, 1H), 1.97-1.88 (m, 1H), 1.87-1.78 (m, 1H), 1.75-1.69 (m, 1H), 1.62 (s, 3H), 1.51-1.46 (m, 3H), 1.40-1.33 (m, 1H).

### Step 7: (S)-methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (A-148) and (R)-methyl 5-(3-6 (1-(3-aminobenzyl)sulfonyl)-2,2-dimethylpiperidin-4-yl)oxy)phenyl)-4-chloro-3-(2-methoxy-2-oxoethoxy)thiophene-2-carboxylate (A-149)

Methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (10, 620 mg, 973.06 mmol) was purified by SFC separation (flow: 70 mL/min; Cycle Time: 5.0 min, total time: 90 min Single; Mobile Phase: 50% IPA+ACN (0.1% NH₃·H₂O) in Supercritical CO₂ over 10 min; injection volume: 2.0 ml Back Pressure: 100 bar to keep the CO₂ in Supercritical flow; column: Daicel ChiralPak IG (250×30 mm, 10 μm)) to give peak 1 as(S)-methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (A-148, 0.3 g, 461.42 μmol) as colorless oil (SFC: Rt=1.090 min, ee.=99.73%) and peak 2 as (R)-methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (A-149, 0.3 g, 456.71 mol) as colorless oil (SFC: Rt=1.695 min, ee.=97.736%).

### tert-butyl 5-[3-[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (A-150A) & tert-butyl 5-[3-[[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (A-150B)

Configurations are arbitrarily assigned.

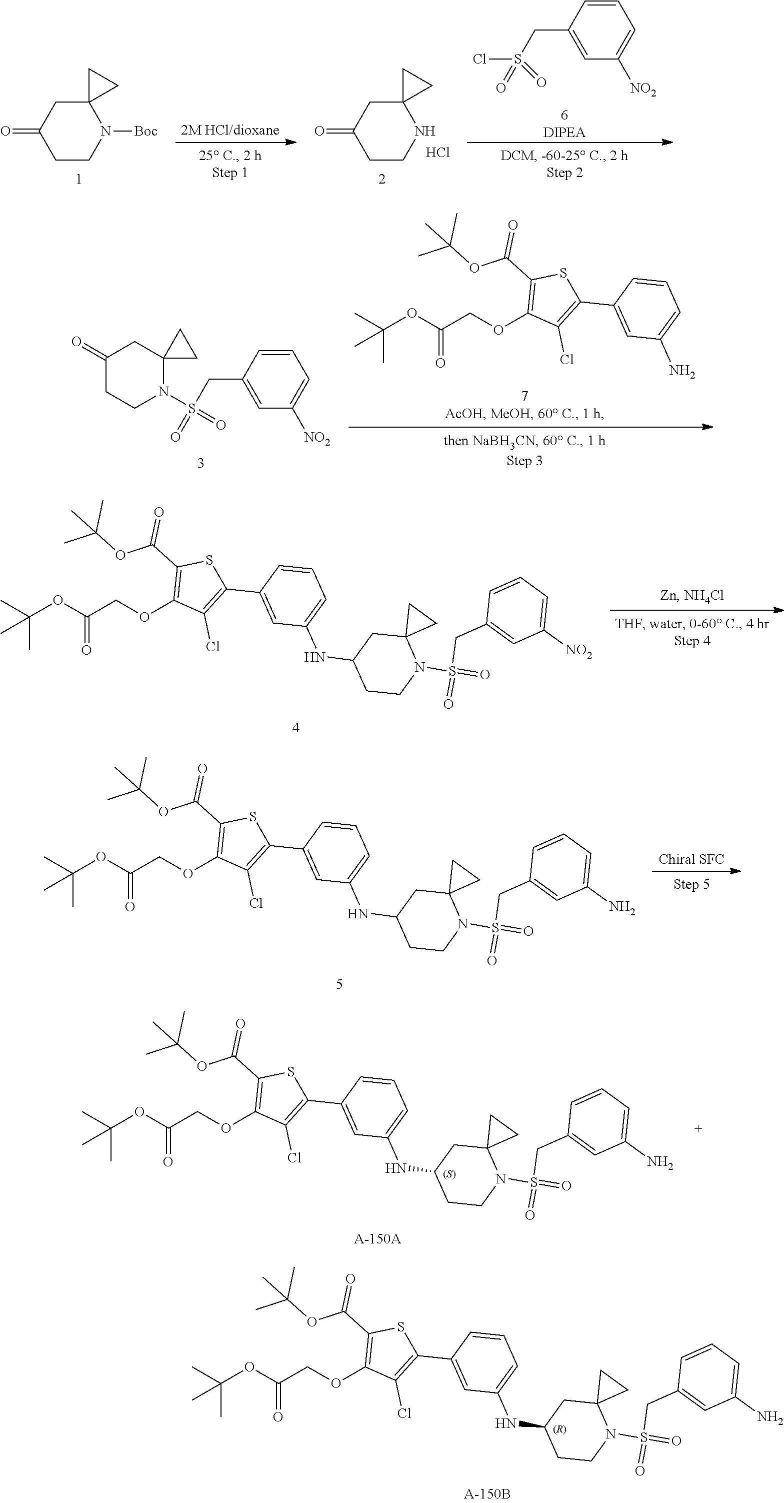

### Step 1: 4-azaspiro[2.5]octan-7-one hydrochloride (2)

4-azaspiro[2.5]octan-7-one (2, 10 g, 61.87 mmol, 99% yield, HCl salt) was synthesized from tert-butyl 7-oxo-4-azaspiro[2.5]octane-4-carboxylate (1, 14 g, 62.14 mmol) in a similar fashion to (6, of step 3) of Compound A-148/Compound A-149, using HCl/dioxane (4 M, 100 mL) and stirring at 25° C. for 2 h. ¹H NMR (400 MHz, DMSO-d6) δ=10.16 (br s, 2H), 3.41-3.37 (m, 2H), 2.68-2.67 (m, 2H), 2.61 (s, 2H), 1.16-1.09 (m, 2H), 0.76-0.69 (m, 2H).

### Step 2: 4-((3-nitrobenzyl)sulfonyl)-4-azaspiro[2.5]octan-7-one (3)

To a solution of 4-azaspiro[2.5]octan-7-one (2, 8 g, 49.50 mmol, HCl salt) in DCM (80 mL) was added DIPEA (14.07 g, 108.89 mmol). Then a suspension of (3-nitrophenyl)methanesulfonyl chloride (6, 12.83 g, 54.45 mmol) in DCM (160 mL) was added drop-wise at −65° C. The reaction mixture was stirred at 25° C. for 2 h, combined with another batch (2 g HCl salt batch) and poured into saturated NH₄Cl aqueous (200 mL) and extracted by DCM (100 mL×2). The combined organic layer was washed with brine, dried over anhydrous sodium sulfate, filtered and concentrated to give a residue. The residue was triturated with EtOH (30 mL), the suspension was stirred at 25° C. for 1 h, then filtered to afford 4-[(3-nitrophenyl)methylsulfonyl]-4-azaspiro[2.5]octan-7-one (3, 15.2 g, 37.49 mmol, 76% yield) as white solid. LCMS (ESI): m/z 325.0 [M+H]⁺.

### Step 3: tert-butyl 3-(2-(tert-butoxy)-2-oxoethoxy)-4-chloro-5-(3-((4-((3-nitrobenzyl)sulfonyl)-4-azaspiro[2,5]octan-7-yl)amino)phenyl)thiophene-2-carboxylate (4)

To a suspension of 4-[(3-nitrophenyl)methylsulfonyl]-4-azaspiro[2.5]octan-7-one (3, 12.2 g, 37.61 mmol) and tert-butyl 5-(3-aminophenyl)-3-(2-tert-butoxy-2-oxo-ethoxy)-4-chloro-thiophene-2-carboxylate (7, 18.20 g, 41.37 mmol) in MeOH (120 mL) was added AcOH (4.52 g, 75.23 mmol, 4.30 mL). The reaction mixture was stirred at 60° C. for 1 h. NaBH₃CN (7.09 g, 112.84 mmol) was added portion-wise at 60° C., and the reaction mixture was stirred at 60° C. for 1 b. The mixture was poured into water (500 mL) and extracted by EtOAc (500 mL×3). The combined organic layer was washed with brine (100 mL), dried over anhydrous sodium sulfate, filtered and concentrated to give a residue. The residue was purified by flash silica gel chromatography (330 g Silica Flash Column, Eluent of 0˜30% Ethyl acetate/Petroleum ether gradient @ 100 mL/min) to afford tert-butyl 3-(2-(tert-butoxy)-2-oxoethoxy)-4-chloro-5-(3-((4-((3-nitrobenzyl)sulfonyl)-4-azaspiro[2.5]octan-7-yl)amino)phenyl)thiophene-2-carboxylate (4, 20 g, 25.66 mmol, 68% yield) as yellow solid.

LCMS (ESI): m/z 748.1 [M+H]⁺.

### Step 4: tert-butyl 5-(3-((4-((3-aminobenzyl)sulfonyl)-4-azaspiro[2.5]octan-7-yl)amino)phenyl)-3-(2-(tert-butoxy)-2-oxoethoxy)-4-chlorothiophene-2-carboxylate (5)

To a solution of tert-butyl tert-butyl 3-(2-(tert-butoxy)-2-oxoethoxy)-4-chloro-5-(3-((4-((3-nitrobenzyl)sulfonyl)-4-azaspiro[2.5]octan-7-yl)amino)phenyl)thiophene-2-carboxylate (4, 9 g, 12.03 mmol) in THF (90 mL) and water (45 mL) was added Zn (15.73 g, 240.54 mmol) in portions at 0° C. The mixture was cooled to 0° C. and NH₄Cl (12.87 g, 240.54 mmol) was added in portions, then the reaction mixture was stirred at 60° C. for 4 h. The reaction mixture was diluted with THF (270 mL), filtered through a pad of Celite, washed with acetone (90 mL) and the filtrate was concentrated under reduced pressure. The residue was taken up in dichloromethane (270 mL), washed with water (90 mL) and brine (90 mL). The organic phase was dried over anhydrous sodium sulfate, filtered and concentrated under reduced pressure. The residue was purified by reversed phased HPLC (formic acid as modifier) and lyophilized to afford tert-butyl 5-(3-((4-((3-aminobenzyl)sulfonyl)-4-azaspiro[2.5]octan-7-yl)amino)phenyl)-3-(2-(tert-butoxy)-2-oxoethoxy)-4-chlorothiophene-2-carboxylate (5, 8 g, 9.58 mmol, 80% yield) as yellow solid. LCMS (ESI): m/z 718.2 [M+H]⁺.

### Step 5; tert-butyl 5-[3-[[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (A-150A) and tert-butyl 5-[3-[[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (A-150B)

tert-butyl 5-[3-[[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (5, 9 g, 12.53 mmol) was separated by SFC (column: Daicel ChiralPak IG (250*30 mm, 10 μm); mobile phase: [0.1% NH₃H₂O MEOH]; B %: 40%-40%, 5.9 min; 2360 min).

Peak 1; tert-butyl 5-[3-[[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (A-150A, 2.3 g, 2.98 mmol, 24% yield) was obtained as light yellow solid.

LCMS (ESI): m/z 718.4 [M+H]⁺.

¹H NMR (400 MHz, DMSO-d6) δ=7.17-7.15 (m, 1H), 6.97-6.95 (m, 1H), 6.84 (s, 1H), 6.80-6.73 (m, 1H), 6.67-6.66 (m, 1H), 6.52-6.50 (m, 1H), 6.46 (s, 1H), 6.39 (d, J=7.6 Hz, 1H), 5.91 (d, J=8.4 Hz, 1H), 5.11 (s, 2H), 4.84 (s, 2H), 4.18-3.99 (m, 2H), 3.56-3.52 (m, 2H), 3.00-2.94 (m, 1H), 1.95 (d, J=10.4 Hz, 1H), 1.74-1.71 (m, 1H), 1.51 (s, 10H), 1.41 (s, 9H), 1.34-1.33 (m, 1H), 0.71-0.60 (m, 2H), 0.53-0.37 (m, 2H).

Peak 2: tert-butyl 5-[3-[[4-[(3-aminophenyl)methylsulfonyl]-4-azaspiro[2.5]octan-7-yl]amino]phenyl]-3-(2-tert-butoxy-2-oxo-ethoxy)-4-chloro-thiophene-2-carboxylate (A-150B, 2.4 g, 3.27 mmol, 26% yield) was obtained as light yellow solid. LCMS (ESI): m/z 718.4 [M+H]⁺.

¹H NMR (400 MHz, DMSO-d6) δ=7.17-7.16 (m, 1H), 6.97-6.95 (m, 1H), 6.84 (s, 1H), 6.78 (d, J=7.6 Hz, 1H), 6.67-6.66 (m, 1H), 6.53-6.52 (m, 1H), 6.46 (s, 1H), 6.38 (d, J=7.6 Hz, 1H), 5.91 (d, J=8.4 Hz, 1H), 5.11 (s, 2H), 4.84 (s, 2H), 4.19-3.99 (m, 2H), 3.60-3.50 (m, 2H), 3.00-2.94 (m, 1H), 1.94 (d, J=10.8 Hz, 1H), 1.79-1.69 (m, 1H), 1.51 (s, 10H), 1.41 (s, 9H), 1.34-1.29 (m, 1H), 0.72-0.57 (m, 2H), 0.52-0.39 (m, 2H).

### 3-[2-oxo-6-(4-piperidylmethyl)benzo[cd]indol-1-yl]piperidine-2,6-dione (B-391)

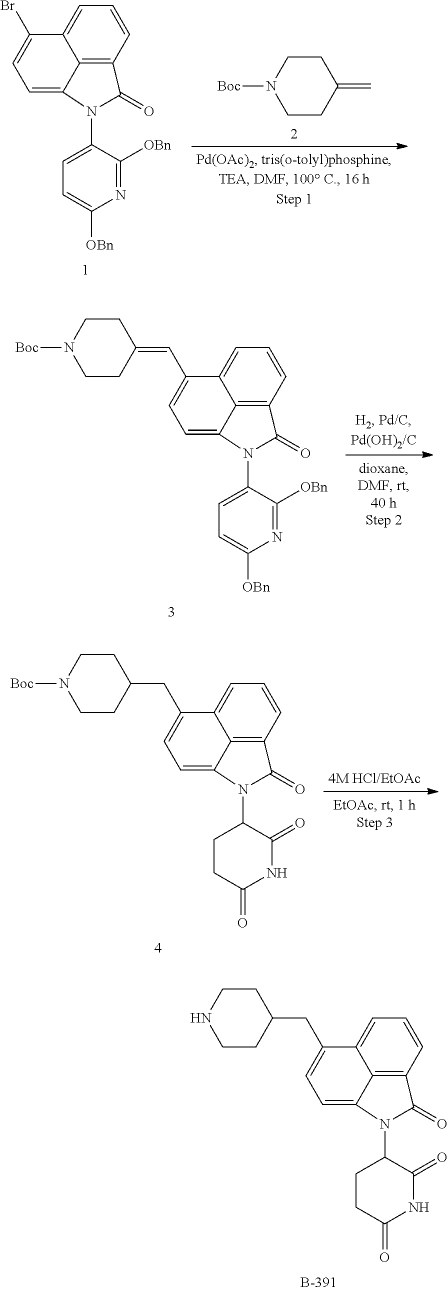

### Step 1: tert-butyl 4-[[1-(2,6-dibenzyloxy-3-pyridyl)-2-oxo-benzo[cd]indol-6-yl]methylene]piperidine-1-carboxylate (3)

To a solution of 1-(2,6-bis(benzyloxy)pyridin-3-yl)-6-bromobenzo[cd]indol-2 (1H)-one (1, 1.2 g, 2.23 mmol) and tert-butyl 4-methylenepiperidine-1-carboxylate (2, 528.60 mg, 2.68 mmol) in DMF (20 mL) was added triethylamine (2.26 g, 22.33 mmol, 3.11 mL) under nitrogen atmosphere. The resulting mixture was degassed with nitrogen for 5 min. Then palladium(II)acetate (50.13 mg, 223.30 μmol) and tris-o-tolylphosphane (101.95 mg, 334.94 μmol) were added and the mixture was degassed for 5 min. The reaction mixture was heated to 100° C. and stirred for 16 h. After being cooled to rt, the mixture was filtered and the filtrate was concentrated under reduced pressure. The residue was purified on automated flash chromatography system (ethyl acetate/petroleum ether from 0:1 to 3:7; Rf=0.5, ethyl acetate/petroleum ether=3/1) to tert-butyl 4-[[1-(2,6-dibenzyloxy-3-pyridyl)-2-oxo-benzo[cd]indol-6-give yl]methylene]piperidine-1-carboxylate (3, 1.1 g, 1.51 mmol, 68% yield) as a brown oil.

LCMS: m/z 654.6 [M+1]⁺

### Step 2: tert-butyl 4-[[1-(2,6-dioxo-3-piperidyl)-2-oxo-benzo[cd]indol-6-yl]methyl]piperidine-1-carboxylate (4)

To a solution of tert-butyl 4-[1-(2,6-dibenzyloxy-3-pyridyl)-2-oxo-benzo[cd]indol-6-yl]methylene]piperidine-1-carboxylate (3, 900 mg, 1.38 mmol) in DMF (20 mL) were added 10 wt. % Pd/C (500 mg) and 20 wt. % Pd(OH)₂/C (500 mg) under nitrogen atmosphere. Then the mixture was stirred at 20° C. for 16 h under H₂ (15 psi). The reaction mixture was filtered through a pad of Celite, and then 10 wt. % Pd/C (500 mg) and 20 wt. % Pd(OH)₂/C (500 mg) were added to the filtrate. The resulting mixture was stirred at 20° C. for additional 24 h. The resulting mixture was filtered through a pad of Celite, and the filtrate was concentrated under reduced pressure. The residue was purified by reversed phase column (0.1% FA in water/acetonitrile, Column: C18, 120 g) and concentrated under reduced pressure to give tert-butyl 4-[[1-(2,6-dioxo-3-piperidyl)-2-oxo-benzo[cd]indol-6-yl]methyl]piperidine-1-carboxylate (4, 360 mg, 746.31 μmol, 54% yield) as a light brown solid.

LCMS (ESI): m/z 422.1 [M−55]⁺

### Step 3: 3-[2-oxo-6-(4-piperidylmethyl)benzo[cd]indol-1-yl]piperidine-2,6-dione (B-391)

To a solution of tert-butyl 4-[1-(2,6-dioxo-3-piperidyl)-2-oxo-benzo[cd]indol-6-yl]methyl]piperidine-1-carboxylate (4, 200 mg, 418.80 μmol) in EtOAc (6 mL) was added HCl/EtOAc (4 M, 6 mL) at 20° C. The mixture was stirred at 20° C. for 1 h. The reaction mixture was concentrated in vacuo to give 3-[2-oxo-6-(4-piperidylmethyl)benzo[cd]indol-1-yl]piperidine-2,6-dione (B-391, 170 mg, 410.73 μmol, 98% yield, HCl salt) as a yellow solid. The material was used in the next step without purification. LCMS (ES+): m/z 378.2 [M+H]⁺.

### 3-(2-oxo-5-(piperidin-4-yl)-3-(tetrahydro-2H-pyran-4-yl)-2,3-dihydro-1H-benzo[d]imidazol-1-yl))piperidine-2,6-dione (B-392)

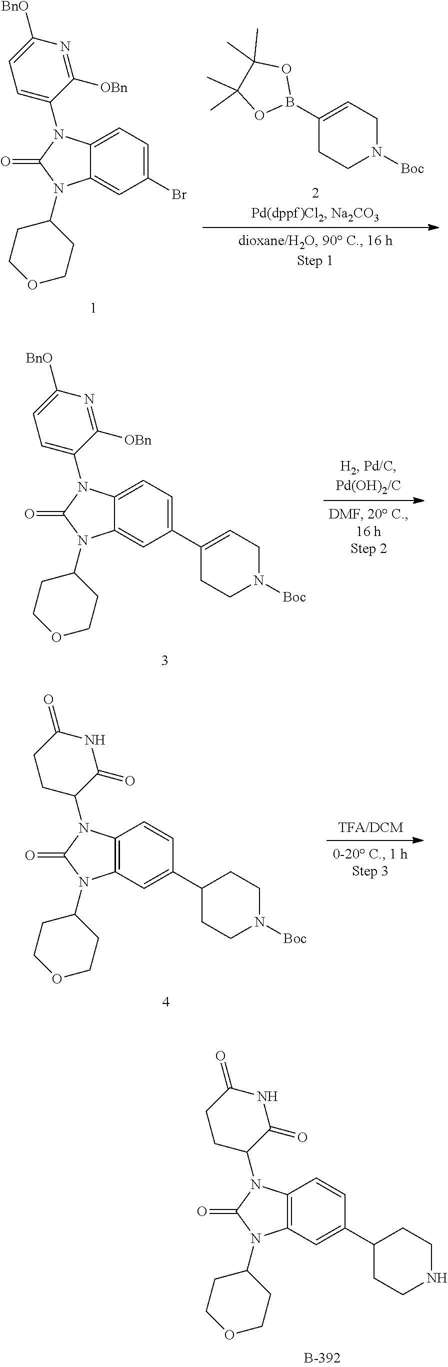

### Step 1: tert-butyl 4-(1-(2,6-bis(benzyloxy)pyridin-3-yl)-2-oxo-3-(tetrahydro-2H-pyran-4-yl)-2,3-dihydro-1H-benzo[d]imidazol-5-yl)-5,6-dihydropyridine-1(2)-carboxylate (3)

To a mixture of 1-(2,6-bis(benzyloxy)pyridin-3-yl)-5-bromo-3-(tetrahydro-2H-pyran-4-yl)-1H-benzo[d]imidazol-2(3H)-one (1, 500 mg, 852.55 μmol), tert-butyl 4-(4,4,5,5-tetramethyl-1,3,2-dioxaborolan-2-yl)-5,6-dihydropyridine-1(2H)-carboxylate (2, 527.23 mg, 1.71 mmol) in dioxane (5 mL), H₂O (1 mL) was added Na₂CO₃ (225.90 mg, 2.13 mmol, 89.29 μL) and Pd(dppf)Cl₂ (62.38 mg, 85.26 μmol). The mixture was stirred at 90° C. for 16 h under N₂. The residue was diluted with H₂O (150 mL) and extracted with ethyl acetate (150 mL×2). The combined organic layers were washed with brine (150 mL×3), dried over Na₂SO₄, filtered and concentrated under reduced pressure. The residue was purified by column chromatography (SiO₂, Petroleum ether:Ethyl acetate=1:0 to 1:1) to afford tert-butyl 4-(1-(2,6-bis(benzyloxy)pyridin-3-yl)-2-oxo-3-(tetrahydro-2H-pyran-4-yl)-2,3-dihydro-1H-benzo[d]imidazol-5-yl)-5,6-dihydropyridine-1(2H)-carboxylate (3, 540 mg, 783.96 μmol, 92% yield) as a white solid. LCMS (ESI): m/z 689.3 [M+H]⁺.

### Step 2: text-butyl 4-(1-(2,6-dioxopiperidin-3-yl)-2-oxo-3-(tetrahydro-2H-pyran-4-yl)-2,3-dihydro-1H-benzo[d]imidazol-5-yl)piperidine-1-carboxylate (4)

tert-butyl 4-(1-(2,6-dioxopiperidin-3-yl)-2-oxo-3-(tetrahydro-2H-pyran-4-yl)-2,3-dihydro-1H-benzo[d]imidazol-5-yl)piperidine-1-carboxylate (4, 130 mg, 253.61 mol, 44% yield) was synthesized from tert-butyl 4-(1-(2,6-bis(benzyloxy)pyridin-3-yl)-2-oxo-3-(tetrahydro-2H-pyran-4-yl)-2,3-dihydro-1H-benzo[d]imidazol-5-yl)-5,6-dihydropyridine-1(2H)-carboxylate (3, 400 mg, 580.71 μmol) in a similar fashion to (4, step 2 of Compound B-391, except using 10 wt. % Pd(OH)₂/C (100 mg,) and 10 wt. % Pd/C (100 mg) and stirring at 20° C. for 16 h under H₂ (15 Psi). The material was purified by column chromatography (SiO₂, Petroleum ether:Ethyl acetate=1:0 to 3:1). LCMS (ESI): m/z 513.5 [M+H]⁺.

### Step 3: 3-(2-oxo-5-(piperidin-4-yl)-3-(tetrahydro-2H-pyran-4-yl)-2,3-dihydro-1H-benzo[d]imidazol-1-yl)piperidine-2,6-dione (B-392)

3-(2-oxo-5-(piperidin-4-yl)-3-(tetrahydro-2H-pyran-4-yl)-2,3-dihydro-1H-benzo[d]imidazol-1-yl)piperidine-2,6-dione (B-392, 120 mg, 227.92 μmol, 89% yield, TFA salt) was synthesized from tert-butyl 4-(1-(2,6-dioxopiperidin-3-yl)-2-oxo-3-(tetrahydro-2H-pyran-4-yl)-2,3-dihydro-1H-benzo[d]imidazol-5-yl)piperidine-1-carboxylate (4, 130 mg, 253.61 μmol) in a similar fashion to Compound A-62, except using 35 eq. of TFA. The material was used in the next step without further characterization.

### 3-[3-(2-methoxyethyl)-2-oxo-5-(4-piperidyl)benzimidazol-1-yl]piperidine-2,6-dione (B-393)

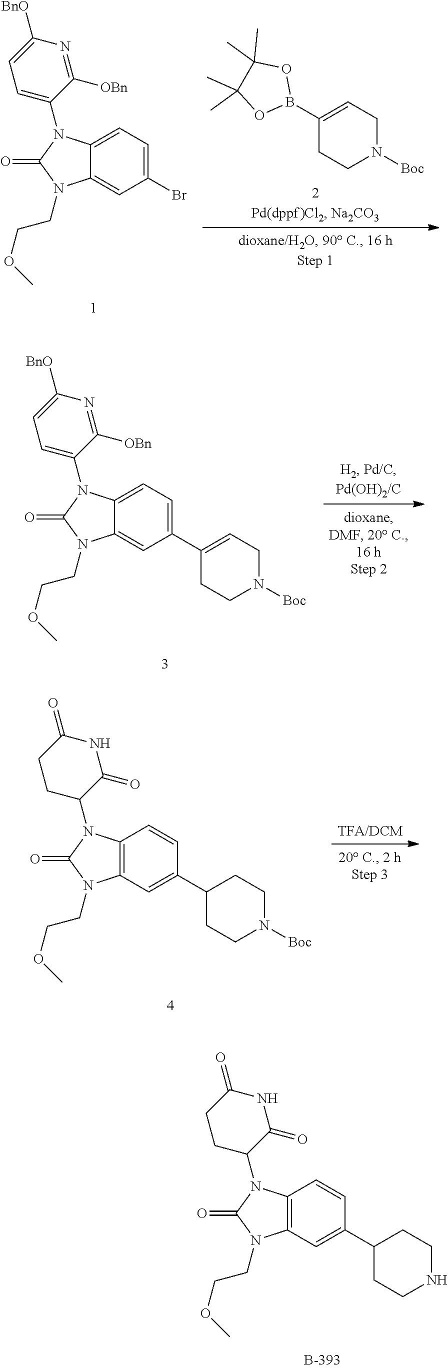

### Step 1: tert-butyl 4-[1-(2,6-dibenzyloxy-3-pyridyl)-3-(2-methoxyethyl)-2-oxo-benzimidazol-5-yl]-3,6-dihydro-2H-pyridine-1-carboxylate (3)

To a solution of 5-bromo-1-(2,6-dibenzyloxy-3-pyridyl)-3-(2-methoxyethyl)benzimidazol-2-one (1, 600 mg, 1.07 mmol) and tert-butyl 4-(4,4,5,5-tetramethyl-1,3,2-dioxaborolan-2-yl)-3,6-dihydro-2H-pyridine-1-carboxylate (396 mg, 1.28 mmol) in water (2 mL) and 1,4-dioxane (10 mL) were added Na₂CO₃ (342 mg, 3.23 mmol) and Pd(dppf)Cl₂ (78 mg, 106.60 μmol) under N₂. The reaction mixture was stirred at 90° C. for 16 b. After the reaction mixture was cooled to rt, ethyl acetate (40 mL) and water (40 mL) were added and layers were separated. The aqueous phase was extracted with ethyl acetate (30 mL). Combined extracts were washed with brine (30 mL), dried over Na₂SO₄, filtered, and concentrated under vacuum. The residue was purified on automated flash chromatography system (ethyl acetate/petroleum ether from 0:1 to 4:6) to afford tert-butyl 4-[1-(2,6-dibenzyloxy-3-pyridyl)-3-(2-methoxyethyl)-2-oxo-benzimidazol-5-yl]-3,6-dihydro-2H-pyridine-1-carboxylate (3, 670 mg, 1.00 mmol, 93% yield) as a white solid. LCMS (ESI): m/z 663.3 [M+H]⁺.

### Step 2: tert-butyl 4-[1-(2,6-dioxo-3-piperidyl)-3-(2-methoxyethyl)-2-oxo-benzimidazol-5-yl]piperidine-1-carboxylate (4)

tert-butyl 4-[1-(2,6-dioxo-3-piperidyl)-3-(2-methoxyethyl)-2-oxo-benzimidazol-5-yl]piperidine-1-carboxylate (4, 410 mg, 834.23 μmol, 83% yield) was synthesized from tert-butyl 4-[1-(2,6-dibenzyloxy-3-pyridyl)-3-(2-methoxyethyl)-2-oxo-benzimidazol-5-yl]-3,6-dihydro-2H-pyridine-1-carboxylate (3, 670 mg, 1.01 mmol) in a similar fashion to (4, step 2 of Compound B-391, except using 10 wt. % Pd/C (300 mg) and 10 wt. % Pd(OH)₂/C (300 mg) and stirring at 20° C. for 16 h under H₂ (15 psi). The material was purified by reversed-phase column (0.1% FA in water/acetonitrile). LCMS (ESI): m/z 487.3 [M+H]⁺.

### Step 3: 3-[3-(2-methoxyethyl)-2-oxo-5-(4-piperidyl)benzimidazol-1-yl]piperidine-2,6-dione (B-393)

3-[3-(2-methoxyethyl)-2-oxo-5-(4-piperidyl)benzimidazol-1-yl]piperidine-2,6-dione (B-393, 400 mg, 759.29 μmol, 90% yield, TFA salt) was synthesized from tert-butyl 4-(1-(2,6-dioxopiperidin-3-yl)-3-(2-methoxyethyl)-2-oxo-2,3-dihydro-1H-benzo[d]imidazol-5-yl)piperidine-1-carboxylate (4, 410 mg, 842.65 μmol) in a similar fashion to Compound A-62, except using 15 eq. TFA. LCMS (ESI): m/z 387.2 [M+H]⁺.

### 3-(6-(1,4-diazepan-1-yl)-2-oxobenzo[cd]indol-1(2H)-yl)piperidine-2,6-dione (B-394)

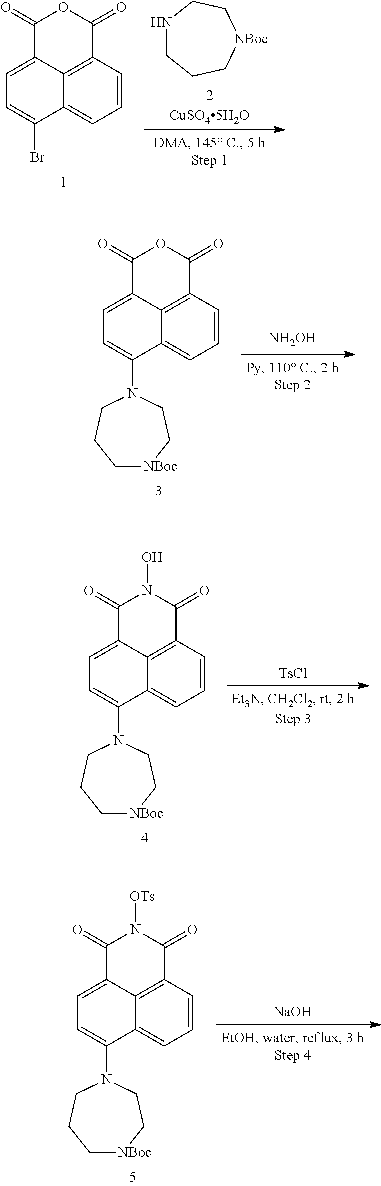

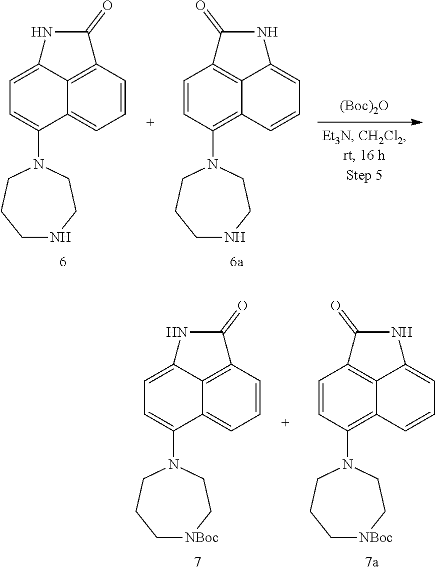 [SMILES: "(Boc)2O" ; O=C1Nc2ccc(N3CCCNCC3)c3cccc1c23 ; O=C1Nc2cccc3c(N4CCCNCC4)ccc1c23 ; CC(C)(C)OC(=O)N1CCCN(c2ccc3c4c(cccc24)C(=O)N3)CC1 ; CC(C)(C)OC(=O)N1CCCN(c2ccc3c4c(cccc24)NC3=O)CC1]

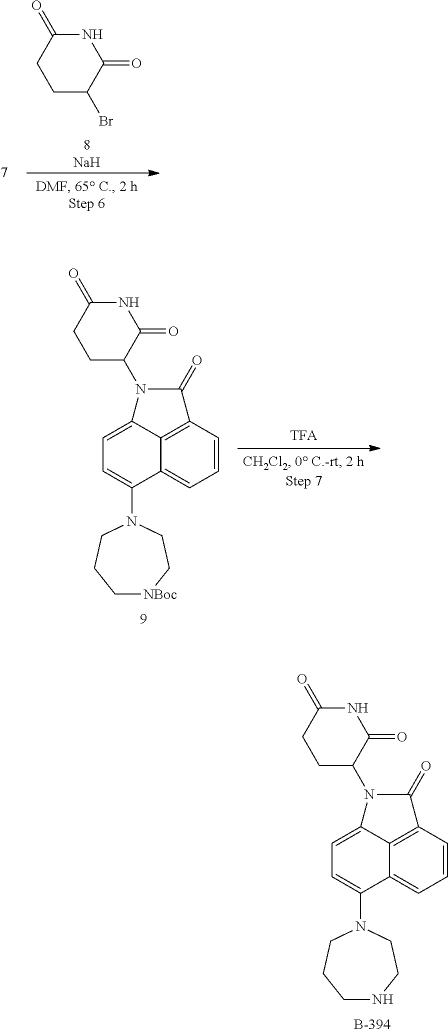

### Step 1: tert-butyl 4-(1,3-dioxo-1H,3H-benzo[de]isochromen-6-yl)-1,4-diazepane-1-carboxylate (3)

Into a 250 ml sealed-tube containing a well-stirred solution of 8-bromo-14-oxatricyclotrideca-2 (6),3,5(8),7(9)-pentaene-10,11-dione (1, 5 g, 18.05 mmol) in dry DMA (30 mL) were added copper(II) sulfate pentahydrate (1.35 g, 5.41 mmol) and tert-butyl 1,4-diazepane-1-carboxylate (2, 5.42 g, 27.07 mmol, 5.31 mL). The reaction mixture was stirred for 5 h at 145° C. The reaction mixture was diluted with water (50 mL) and extracted with EtOAc (2×100 mL). The combined organic layers were dried over Na₂SO₄, filtered, concentrated and purified by silica gel (100 g, Silica 60-120 mesh) flash column chromatography (30%-60% EtOAc in Pet-ether) to afford tert-butyl 4-(1,3-dioxo-1H,3H-benzo[de]isochromen-6-yl)-1,4-diazepane-1-carboxylate (3, 4 g, 6.44 mmol, 36% yield, 64% purity) as yellow solid.

LCMS (ES+): m/z 341.2 [M−tBu+H]⁺

### Step 2: tert-butyl 4-(2-hydroxy-1,3-dioxo-2,3-dihydro-1H-benzo[de]isoquinolin-6-yl)-1,4-diazepane-1-carboxylate (4)

Into a 250 mL round bottom flask containing a well-stirred solution of tert-butyl 4-(1,3-dioxo-1H,3H-benzo[de]isochromen-6-yl)-1,4-diazepane-1-carboxylate (3, 4 g, 6.46 mmol) in pyridine (30 mL) was added hydroxylamine hydrochloride (673.10 mg, 9.69 mmol). The reaction mixture was stirred at 110° C. for 2 h. The mixture was concentrated under reduced pressure to afford tert-butyl 4-(2-hydroxy-1,3-dioxo-benzo[de]isoquinolin-6-yl)-1,4-diazepane-1-carboxylate (4, 2.8 g, 6.27 mmol, 97% yield) as yellow solid.

LCMS (ES+): m/z 412.2 [M+H]⁺.

### Step 3: tert-butyl 4-(1,3-dioxo-2-(tosyloxy)-2,3-dihydro-1H-benzo[de]isoquinolin-6-yl)-1,4-diazepane-1-carboxylate (5)

Into a 250 mL round bottom flask containing a well-stirred solution of tert-butyl 4-(2-hydroxy-1,3-dioxo-benzo[de]isoquinolin-6-yl)-1,4-diazepane-1-carboxylate (4, 2.8 g, 6.26 mmol) in DCM (40 mL) was added Et₃N (1.27 g, 12.52 mmol, 1.75 mL) and TsCl (1.43 g, 7.51 mmol). After 2 h, water (30 mL) was added to the reaction mixture and extracted with DCM (2×100 mL). The combined organic layers were dried over sodium sulfate, concentrated and purified by silica gel (100 g, 60-120 mesh) column chromatography (20% EtOAc in pet-ether) to afford tert-butyl 4-(1,3-dioxo-2-(tosyloxy)-2,3-dihydro-1H-benzo[de]isoquinolin-6-yl)-1,4-diazepane-1-carboxylate (5, 2.8 g, 3.04 mmol, 49% yield, 61% purity) as an orange solid.

LCMS (ES+): m/z 510.0 [M−tBu+H]⁻.

### Step 4:6-(1,4-diazepan-1-yl)benzo[cd]indol-2 (1H)-one (6)

Into a 250 mL single neck round bottom flask containing a suspension of tert-butyl 4-(1,3-dioxo-2-(tosyloxy)-2,3-dihydro-1H-benzo[de]isoquinolin-6-yl)-1,4-diazepane-1-carboxylate (5, 2.8 g, 3.17 mmol) in ethanol (20 mL) and water (10 mL) was added 10% NaOH solution (1.27 g, 31.68 mmol, 20 mL) and the resulting mixture was refluxed for 3 h. Ethanol was evaporated, and the mixture was carefully neutralized by adding 6 N HCl dropwise. The mixture was concentrated under reduced pressure to afford a mixture of inseparable regioisomers 6-(1,4-diazepan-1-yl)-benzo[cd]indol-2 (1H)-one and 5-(1,4-diazepan-1-yl)benzo[cd]indol-2 (1H)-one (6+6a, 900 mg, 2.48 mmol, 78% yield, HCl salt) as orange gummy solid.

LCMS (ES+): m/z 268.2 [M+H]⁺.

### Step 5: tert-butyl 4-(2-oxo-1,2-dihydrobenzo[cd]indol-6-yl)-1,4-diazepane-1-carboxylate (7) and tert-butyl 4-(2-oxo-1,2-dihydrobenzo[cd]indol-5-yl)-1,4-diazepane-1-carboxylate (7a)

Into a 250 mL two neck round bottom flask containing a well-stirred solution of a mixture of 6-(1,4-diazepan-1-yl)-benzo[cd]indol-2 (1H)-one and 5-(1,4-diazepan-1-yl)benzo[cd]indol-2 (1H)-one (6+6a, 900 mg, 2.48 mmol, 78% yield, HCl salt) in DCM (40 mL) at 0° C. were added Et₃N (1.24 g, 12.29 mmol, 1.71 mL) and di-tert-butyl dicarbonate (590.34 mg, 2.70 mmol, 620.75 μL). The resulting reaction mixture was stirred at rt for 16 h. Then, 50 mL of water was added to the reaction mixture and extracted twice with DCM (2×100 mL). The combined organic layers were dried over anhydrous Na₂SO₄, filtered, and concentrated and purified by silica gel (230-400 mesh) column chromatography (30% EtOAc in pet ether) to afford tert-butyl 4-(2-oxo-1,2-dihydrobenzo[cd]indol-6-yl)-1,4-diazepane-1-carboxylate (7, 700 mg, 1.71 mmol, 70% yield) as orange solid

LCMS (ES+): m/z 368.2 [M+H]⁺

and tert-butyl 4-(2-oxo-1,2-dihydrobenzo[cd]indol-5-yl)-1,4-diazepane-1-carboxylate (7a, 320 mg, 643.59 μmol, 26% yield, 74% purity) as yellow solid.

LCMS (ES+): m/z 368.2 [M+H]⁺.

### Step 6: tert-butyl 4-(1-(2,6-dioxopiperidin-3-yl)-2-oxo-1,2-dihydrobenzo[cd]indol-6-yl)-1,4-diazepane-1-carboxylate (9)

Into a 50 mL two neck round bottom flask containing a well-stirred suspension of sodium hydride (60% dispersion in mineral oil) (375.41 mg, 9.80 mmol) in anhydrous THF (4 mL) at 0° C. was added a solution of tert-butyl 4-(2-oxo-1H-benzo[cd]indol-6-yl)-1,4-diazepane-1-carboxylate (7, 400 mg, 979.75 μmol) in dry THF (4 mL) and the resulting mixture was stirred for 1 h at rt. 3-bromopiperidine-2,6-dione (8, 752.49 mg, 3.92 mmol) in THF (4 mL) was added at 0° C. and the mixture was heated at 65° C. for 2 b. The reaction mixture was quenched with saturated ammonium chloride and extracted with EtOAc (3×50 mL). The combined organic layers were dried over sodium sulfate and concentrated under reduced pressure to afford tert-butyl 4-(1-(2,6-dioxopiperidin-3-yl)-2-oxo-1,2-dihydrobenzo[cd]indol-6-yl)-1,4-diazepane-1-carboxylate (9, 300 mg, 626.28 μmol, 64% yield) as a red colored solid.

LCMS (ES+): 479.0 [M+H]⁺.

### Step 7: 3-(6-(1,4-diazepan-1-yl)-2-oxobenzo[cd]indol-1(2H)-yl)piperidine-2,6-dione (B-394)

3-[6-(1,4-diazepan-1-yl)-2-oxobenzo[cd]indol-1(2H)-yl]piperidine-2,6-dione (B-394, 200 mg, 430.06 μmol, 62% yield, TFA salt) was synthesized from tert-butyl 4-[1-(2,6-dioxo-3-piperidyl)-2-oxo-benzo[cd]indol-6-yl]-1,4-diazepane-1-carboxylate (9, 270 mg, 513.44 μmol) in a similar fashion to Compound A-62, except using 10 eq. TFA and triturating with diethyl ether. LCMS (ES+): m/z 379.0 [M+H]⁺.

### 3-(5-(1,4-diazepan-1-yl)-2-oxobenzo[cd]indol-1(2H)-yl)piperidine-2,6-dione B-394a)

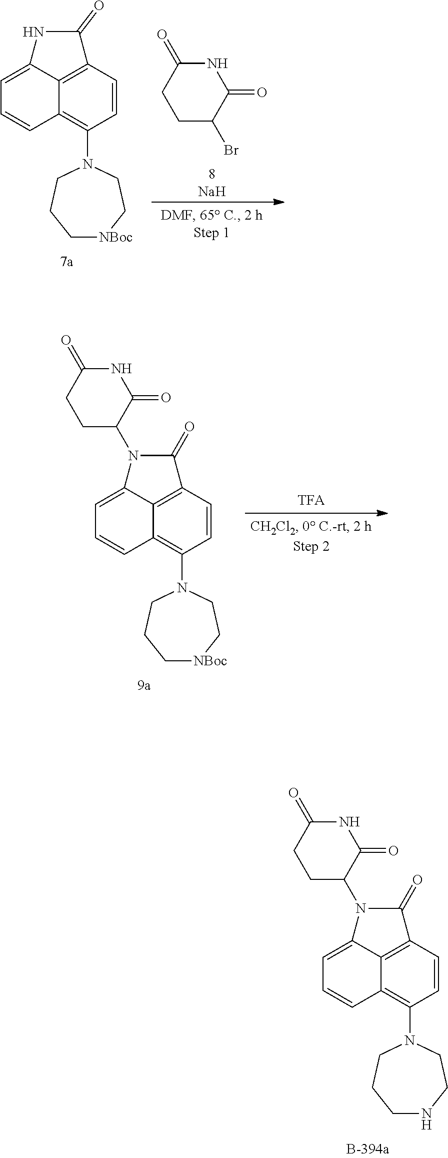

### Step 1: tert-butyl 4-(1-(2,6-dioxopiperidin-3-yl)-2-oxo-1,2-dihydrobenzo[cd]indol-5-yl)-1,4-diazepane-1-carboxylate (9a)

Into a 25 mL two neck round bottom flask containing a well-stirred suspension of sodium hydride (60% dispersion in mineral oil, 146.62 mg, 3.83 mmol, 60% purity) in anhydrous DMF (2 mL) at 0° C. was added a solution of tert-butyl 4-(2-oxo-1H-benzo[cd]indol-5-yl)-1,4-diazepane-1-carboxylate (7a, 380 mg, 765.29 μmol) in dry DMF (2 mL) and the resulting mixture was stirred for 1 h at rt. 3-bromopiperidine-2,6-dione (8, 734.72 mg, 3.83 mmol) in THE (4 mL) was added at 0° C. and the mixture was heated at 65° C. for 2 b. The reaction mixture was quenched with saturated ammonium chloride and extracted with EtOAc (3×50 mL). The combined organic layers were dried over sodium sulfate and concentrated under reduced pressure and purified by silica gel (230-400 mesh silica gel, 25 g) flash column chromatography (50% EtOAc in Pet ether) to afforded tert-butyl 4-(1-(2,6-dioxopiperidin-3-yl)-2-oxo-1,2-dihydrobenzo[cd]indol-5-yl)-1,4-diazepane-1-carboxylate (9a, 150 mg, 308.56 μmol, 40% yield, 71% purity) as yellow solid.

LCMS (ES+): 423.2 [M−tBu+H]⁺.

### Step 2: 3-(5-(1,4-diazepan-1-yl)-2-oxobenzo[cd]indol-1(2H)-yl)piperidine-2,6-dione (B-394a)

3-(5-(1,4-diazepan-1-yl)-2-oxobenzo[cd]indol-1(2H)-yl)piperidine-2,6-dione (B-394a, 110 mg, 252.42 μmol, 72% yield, TFA salt) was synthesized from tert-butyl 4-(1-(2,6-dioxopiperidin-3-yl)-2-oxo-1,2-dihydrobenzo[cd]indol-5-yl)-1,4-diazepane-1-carboxylate (9a, 130 mg, 264.60 μmol) in a similar fashion to Compound A-62, except using 10 eq. TFA and triturating with diethyl ether. LCMS (ES+): m/z 379.2 [M+H]⁺.

### (1r,4r)-4-(1-(2,6-dioxopiperidin-3-yl)-2-oxo-1,2-dihydrobenzo[cd]indol-6-yl)cyclohexane-1-carboxylic acid (B-395a, First Eluted Isomer) and (1s,4s)-4-(1-(2,6-dioxopiperidin-3-yl)-2-oxo-1,2-dihydrobenzo[cd]indol-6-yl)cyclohexane-1-carboxylic acid (B-395b, Second Eluted Isomer)

Configurations are arbitrarily assigned.

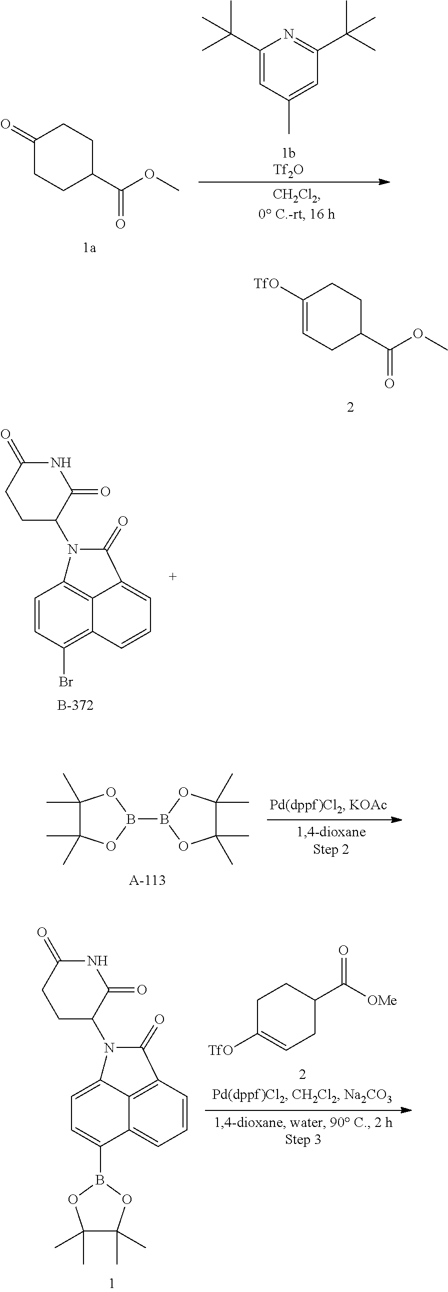

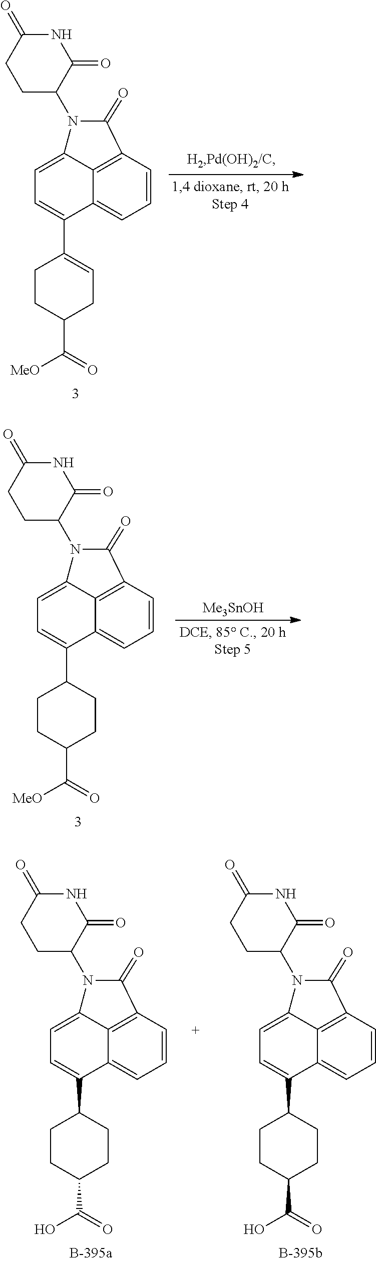 [SMILES: "Me3SnOH" ; COC(=O)C1CC=C(c2ccc3c4c(cccc24)C(=O)N3C2CCC(=O)NC2=O)CC1 ; COC(=O)C1CCC(c2ccc3c4c(cccc24)C(=O)N3C2CCC(=O)NC2=O)CC1 ; O=C1CCC(N2C(=O)c3cccc4c3c2ccc4[C@H]2CC[C@H](C(=O)O)CC2)C(=O)N1 ; O=C1CCC(N2C(=O)c3cccc4c3c2ccc4[C@H]2CC[C@@H](C(=O)O)CC2)C(=O)N1]

### Step 1: methyl 4-(trifluoromethylsulfonyloxy)cyclohex-3-ene-1-carboxylate (2)

Into a 100 mL single neck round bottom flask containing a well-stirred solution of methyl 4-oxocyclohexanecarboxylate (1a, 2 g, 12.81 mmol) in dry DCM (20 mL) was added 2,6-di-tert-butyl-4-methylpyridine (1b, 3.02 g, 14.73 mmol) under nitrogen atmosphere. After 30 min, trifluoromethanesulfonic anhydride (3.79 g, 13.45 mmol, 2.26 mL) was added to the reaction mixture at 0° C. and stirring was continued at rt for 16 h. The reaction mixture was quenched with saturated NaHCO₃ solution and extracted with DCM (2×50 mL). The combined organic layers were dried over anhydrous Na₂SO₄, filtered, concentrated and purified by silica gel flash column chromatography (100-200 mesh silica gel; 10-20% EtOAc in pet-ether) to afford methyl 4-(trifluoromethylsulfonyloxy)cyclohex-3-ene-1-carboxylate (2, 1.8 g, 5.62 mmol, 44% yield) as a yellow oil.

### Step 2: 3-[2-oxo-6-(4,4,5,5-tetramethyl-1,3,2-dioxaborolan-2-yl)benzo[cd]indol-1-yl]piperidine-2,6-dione (1)

Into a 100 mL sealed tube containing a well-stirred solution of 3-(6-bromo-2-oxo-benzo[cd]indol-1-yl) piperidine-2,6-dione (2.5 g, 5.43 mmol) in dry 1,4-dioxane (30 mL) was added KOAc (1.60 g, 16.29 mmol) and bis(pinacolato)diboron (1.51 g, 5.95 mmol) under nitrogen atmosphere and the resulting mixture was degassed by bubbling nitrogen gas through for 5 min. Subsequently, 1,1′-bis(diphenylphosphino)ferrocene-palladium(II)dichloride dichloromethane complex (443.13 mg, 542.91 μmol) was added and the mixture heated at 90° C. for 16 h. The reaction mixture was filtered through Celite and washed with EtOAc (100 mL). Filtrate was concentrated and purified by silica gel column chromatography (100-200 mesh-100 g, 50-70% EtOAc in pet-ether) to afford 3-[2-oxo-6-(4,4,5,5-tetramethyl-1,3,2-dioxaborolan-2-yl)benzo[cd]indol-1-yl]piperidine-2,6-dione (1, 2.0 g, 3.06 mmol, 56% yield, 62% purity) as a yellow color solid.

LCMS (ES+): m/z 407.2 [M+H]⁺

### Step 3: methyl 4-[1-(2,6-dioxo-3-piperidyl)-2-oxo-benzo[cd]indol-6-yl]cyclohex-3-ene-1-carboxylate (3)

Into a 100 mL sealed tube containing a well-stirred solution of 3-[2-oxo-6-(4,4,5,5-tetramethyl-1,3,2-dioxaborolan-2-yl)benzo[cd]indol-1-yl]piperidine-2,6-dione (1, 2.0 g, 3.05 mmol) and methyl 4-(trifluoromethylsulfonyloxy)cyclohex-3-ene-1-carboxylate (2, 1.08 g, 3.36 mmol) in a mixture of 1,4-dioxane (15 mL) and water (1 mL) was added sodium carbonate (323.52 mg, 3.05 mmol) and the mixture was degassed by bubbling nitrogen for 5 min. Finally, Pd(dppf)Cl₂·CH₂Cl₂ (249.14 mg, 305.24 μmol) was added, and the mixture was heated at 90° C. for 2 h. The reaction mixture was filtered through Celite and washed with EtOAc (50 mL). The filtrate was concentrated and purified by silica gel flash column chromatography (230-400 mesh; 50-70% EtOAc in pet ether) to afford methyl 4-[1-(2,6-dioxo-3-piperidyl)-2-oxo-benzo[cd]indol-6-yl]cyclohex-3-ene-1-carboxylate (3, 1.4 g, 2.18 mmol, 72% yield, 65% purity) as a yellow solid.

LCMS (ES+): m/z 419.2 [M+H]⁺.

### Step 4: methyl 4-[1-(2,6-dioxo-3-piperidyl)-2-oxo-benzo[cd]indol-6-yl]cyclohexanecarboxylate (4)

Methyl 4-[1-(2,6-dioxo-3-piperidyl)-2-oxo-benzo[cd]indol-6-yl]cyclohexanecarboxylate (4, 1.3 g, 2.16 mmol, 99% yield, 70% purity) was synthesized from methyl 4-[1-(2,6-dioxo-3-piperidyl)-2-oxo-benzo[cd]indol-6-yl]cyclohex-3-ene-1-carboxylate (3, 1.4 g, 2.18 mmol) in a similar fashion to Compound B-103, except using 20 wt. % palladium hydroxide on carbon, 50% water (1.38 g, 1.97 mmol) and triturating the product with n-hexane. LCMS (ES+): m/z 421.1 [M+H]⁺.

### Step 5: (1r,4r)-4-(1-(2,6-dioxopiperidin-3-yl)-2-oxo-1,2-dihydrobenzo[cd]indol-6-yl)cyclohexane-1-carboxylic acid (B-395a, first eluted isomer) and (1s,4s)-4-(1-(2,6-dioxopiperidin-3-yl)-2-oxo-1,2-dihydrobenzo[cd]indol-6-yl)cyclohexane-1-carboxylic acid (B-395b, second eluted isomer)

Configurations are arbitrarily assigned.

(1r,4r)-4-(1-(2,6-dioxopiperidin-3-yl)-2-oxo-1,2-dihydrobenzo[cd]indol-6-yl)cyclohexane-1-carboxylic acid (B-395a, 25 mg, 53.27 μmol, 13% yield, rt 1.204) and (1s,4s)-4-(1-(2,6-dioxopiperidin-3-yl)-2-oxo-1,2-dibydrobenzo[cd]indol-6-yl)cyclohexane-1-carboxylic acid (B-395b, 25 mg, 45.89 μmol, 11% yield, 75% purity, rt 1.379) were synthesized from methyl 4-[1-(2,6-dioxo-3-piperidyl)-2-oxo-benzo[cd]indol-6-yl]cyclohexanecarboxylate (4, 250 mg, 416.21 μmol) in a similar fashion to Compound B-11, except heating at 85° C. The material was purified by reverse-phase preparative HPLC [Column: SUNFIRE C18 5 MICRON 19×150 mm; Mobile phase A: 0.1% TFA in water and Mobile phase B: MeCN].

LCMS (ESI): m/z 407.1 [M+H]⁺.

### 3-[5-(3-fluoro-4-piperidyl)-3-methyl-2-oxo-benzimidazol-1-yl]piperidine-2,6-dione (B-396)

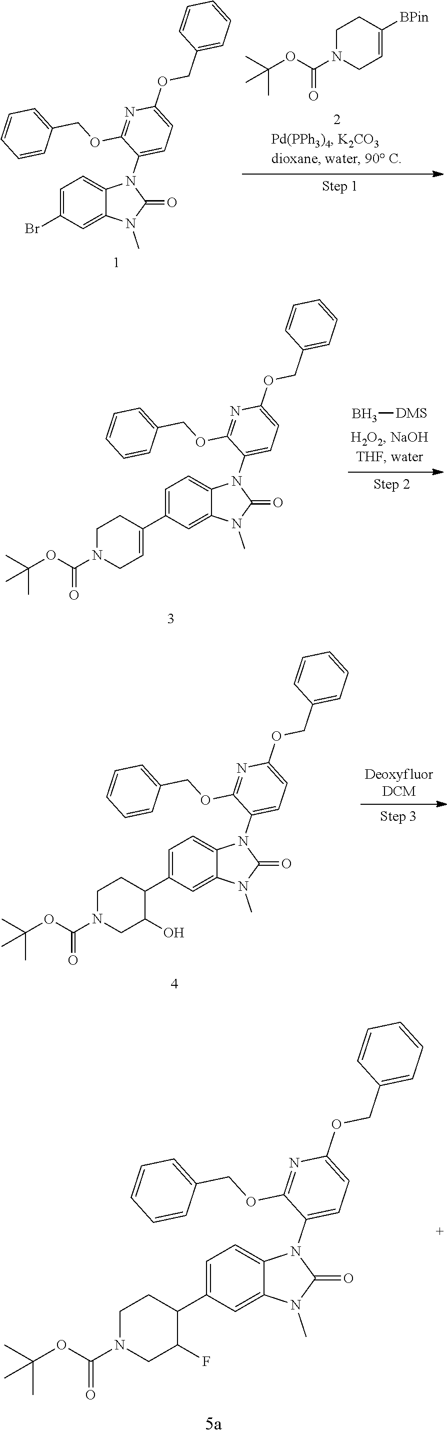

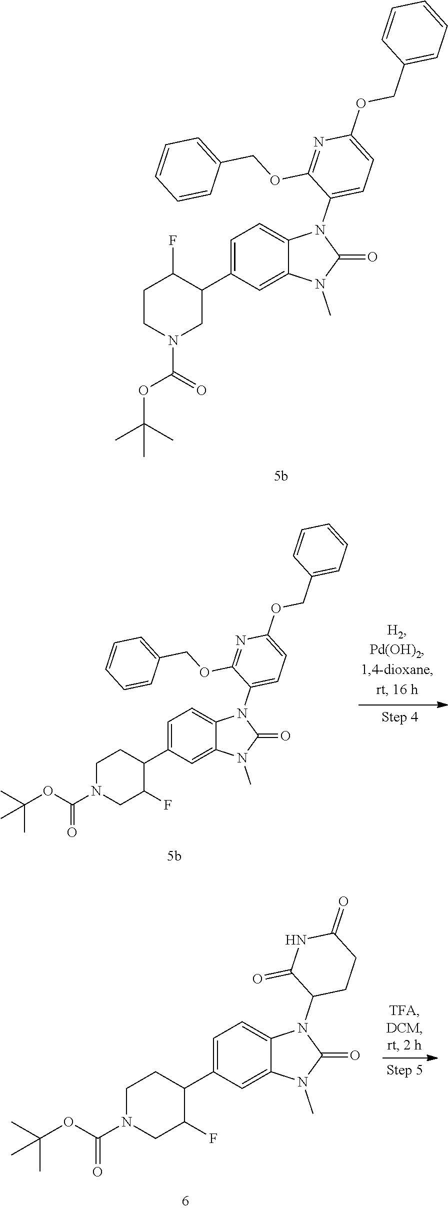 [SMILES: Cn1c(=O)n(-c2ccc(OCc3ccccc3)nc2OCc2ccccc2)c2ccc(C3CN(C(=O)OC(C)(C)C)CCC3F)cc21 ; Cn1c(=O)n(-c2ccc(OCc3ccccc3)nc2OCc2ccccc2)c2ccc(C3CCN(C(=O)OC(C)(C)C)CC3F)cc21 ; Cn1c(=O)n(C2CCC(=O)NC2=O)c2ccc(C3CCN(C(=O)OC(C)(C)C)CC3F)cc21]

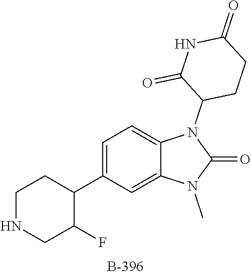 [SMILES: Cn1c(=O)n(C2CCC(=O)NC2=O)c2ccc(C3CCNCC3F)cc21]

### Step 1: tert-butyl 4-[1-(2,6-dibenzyloxy-3-pyridyl)-3-methyl-2-oxo-benzimidazol-5-yl]-3,6-dihydro-2H-pyridine-1-carboxylate (3)

To a mixture of 5-bromo-1-(2,6-dibenzyloxy-3-pyridyl)-3-methyl-benzimidazol-2-one (1, 20 g, 38.73 mmol) and tert-butyl 4-(4,4,5,5-tetramethyl-1,3,2-dioxaborolan-2-yl)-3,6-dihydro-2H-pyridine-1-carboxylate (2, 15.57 g, 50.35 mmol) in 1,4-dioxane (160 mL) and Water (40 mL) was added Potassium carbonate (16.06 g, 116.19 mmol). The resulting mixture was purged with argon gas for 30 min. Pd(PPh₃)₄ (4.48 g, 3.87 mmol) was added and the mixture was heated to reflux at 90° C. for 12 h. The mixture was filtered through Celite and concentrated and water (200 mL) was added. The mixture was extracted with dichloromethane (3×200 mL), concentrated and purified via silica gel column chromatography (eluting with ethyl acetate in Pet ether) to afford tert-butyl 4-[1-(2,6-dibenzyloxy-3-pyridyl)-3-methyl-2-oxo-benzimidazol-5-yl]-3,6-dihydro-2H-pyridine-1-carboxylate (3, 18.0 g, 27.93 mmol, 72% yield) as a brown viscous material. LCMS (ES+): m/z 619.5 [M+H]⁺.

### Step 2: tert-butyl 4-[1-(2,6-dibenzyloxy-3-pyridyl)-3-methyl-2-oxo-benzimidazol-5-yl]-3-hydroxy-piperidine-1-carboxylate (4)

To a stirred a solution of tert-butyl 4-[1-(2,6-dibenzyloxy-3-pyridyl)-3-methyl-2-oxo-benzimidazol-5-yl]-3,6-dihydro-2H-pyridine-1-carboxylate (3, 18 g, 29.09 mmol) in THF (1800 mL) was added Borane dimethyl sulfide complex (11.05 g, 145.45 mmol, 13.80 mL) at 0° C. under argon atmosphere. After 12 h, water (200 mL) was added at 0° C. dropwise, followed by the addition of sodium hydroxide (1N) (5.82 g, 145.46 mmol) dissolved in Water (200 mL). The resulting mixture was stirred at 0° C. for 20 min. Hydrogen peroxide (35%) (4.95 g, 145.46 mmol, 4.50 mL) was added and the resulting mixture was stirred at rt for 4 h. The reaction mixture was diluted with water (250 mL) and extracted with DCM (200 mL×3). The combined organic layer was dried under high vacuum. The material was purified by column chromatography (ethyl acetate in Petroleum ether) to afford tert-butyl 4-[1-(2,6-dibenzyloxy-3-pyridyl)-3-methyl-2-oxo-benzimidazol-5-yl]-3-hydroxy-piperidine-1-carboxylate (4, 15.5 g, 23.86 mmol, 82% yield) as an off white solid. LCMS (ES+): m/z 659.4 [M+Na]⁺

### Step 3: tert-butyl 4-[1-(2,6-dibenzyloxy-3-pyridyl)-3-methyl-2-oxo-benzimidazol-5-yl]-3-fluoro-piperidine-1-carboxylate (5a)

To a stirred solution of tert-butyl 4-[1-(2,6-dibenzyloxy-3-pyridyl)-3-methyl-2-oxo-benzimidazol-5-yl]-3-hydroxy-piperidine-1-carboxy late (4, 15.5 g, 24.34 mmol) in DCM (800 mL) was added Deoxyflour (10.77 g, 48.69 mmol, 8.98 mL) at −70° C. After 3 h, the mixture was diluted with water (200 mL) and quenched with saturated sodium bicarbonate solution (200 mL) and extracted with ethyl acetate (3×150 mL). The combined organic layers were concentrated and the residue purified by column chromatography (ethyl acetate in pet ether) to afford a mixture (5a/5b, 11 g, 9.84 mmol, 40% yield) as a viscous oil. LCMS (ES+): m/z 639.6 [M+H]+.

The mixture of 5a/5b was separated by Prep HPLC purification to afford tert-butyl 4-(1-(2,6-bis(benzyloxy)pyridin-3-yl)-3-methyl-2-oxo-2,3-dihydro-1H-benzo[d]imidazol-5-yl)-3-fluoropiperidine-1-carboxy late (5b, 5.030 g, 7.83 mmol, 62% yield) (Peak-1) and tert-butyl 4-(1-(2,6-bis(benzyloxy)pyridin-3-yl)-3-methyl-2-oxo-2,3-dihydro-1H-benzo[d]imidazol-5-yl)-3-fluoropiperidine-1-carboxylate (5a, 2.56 g, 3.95 mmol, 32% yield) (Peak-2) Mobile Phase (A): 5 mM Ammonium Acetate in H2O Mobile Phase (B): 100% ACETONITRILE Flow Rate: 25 ml/min Column: X-SELECT C18 10 μm (25×150 mm) Gradient Time % B: 0/65, 28/65, 28.1/100, 30/100, 30.1/65, 32/65.

### Step 4: tert-butyl 4-[1-(2,6-dioxo-3-piperidyl)-3-methyl-2-oxo-benzimidazol-5-yl]-3-fluoro-piperidine-1-carboxylate (6)

tert-butyl 4-[1-(2,6-dioxo-3-piperidyl)-3-methyl-2-oxo-benzimidazol-5-yl]-3-fluoro-piperidine-1-carboxylate (6, 470 mg, 994.41 μmol, 91% yield) was synthesized from tert-butyl 4-[1-(2,6-dibenzyloxy-3-pyridyl)-3-methyl-2-oxo-benzimidazol-5-yl]-3-fluoro-piperidine-1-carboxylate (5a, 700 mg, 1.10 mmol) in a similar fashion to Compound B-103, except using 20 wt. % palladium hydroxide on carbon (560.00 mg, 797.52 μmol). The material was used in the next step without further purification. LCMS (ES+): m/z 405.2 [M−tBu+H]⁺.

### Step 5: 3-[5-(3-fluoro-4-piperidyl)-3-methyl-2-oxo-benzimidazol-1-yl]piperidine-2,6-dione (B-396)

3-[5-(3-fluoro-4-piperidyl)-3-methyl-2-oxo-benzimidazol-1-yl]piperidine-2,6-dione (B-396, 420 mg, 865.13 μmol, 87% yield, TFA salt) was synthesized from tert-butyl4-[1-(2,6-dioxo-3-piperidyl)-3-methyl-2-oxo-benzimidazol-5-yl]-3-fluoro-piperidine-1-carboxylate (6, 470 mg, 994.41 μmol) in a similar fashion to Compound A-62, except using 13 eq. TFA and triturating with MTBE. LCMS (ES+): m/z 361.1 [M+H]⁺.

### tert-butyl (3R,4R)-4-[1-(2,6-dibenzyloxy-3-pyridyl)-3-methyl-2-oxo-benzimidazol-5-yl]-3-fluoro-piperidine-1-carboxylate (B-397) and tert-butyl (3S,4S)-4-[1-(2,6-dibenzyloxy-3-pyridyl)-3-methyl-2-oxo-benzimidazol-5-yl]-3-fluoro-piperidine-1-carboxylate (B-398)

Configurations are arbitrarily assigned.

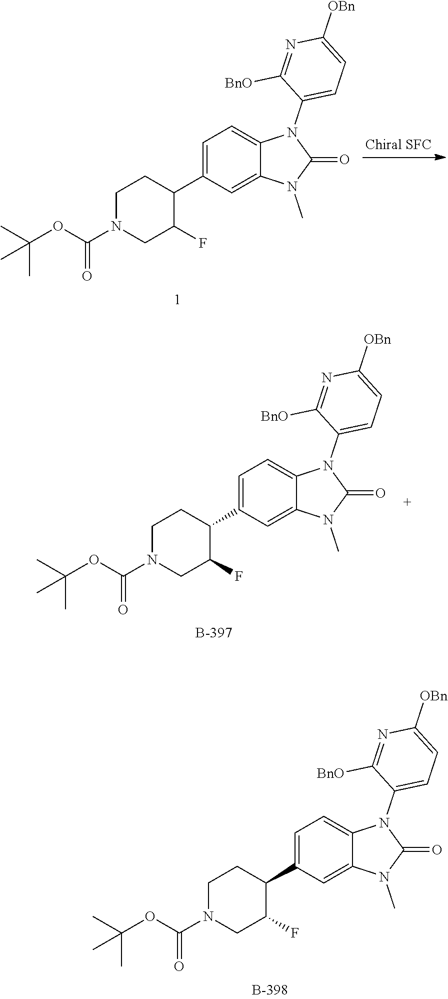 [SMILES: Cn1c(=O)n(-c2ccc(OCc3ccccc3)nc2OCc2ccccc2)c2ccc(C3CCN(C(=O)OC(C)(C)C)CC3F)cc21 ; Cn1c(=O)n(-c2ccc(OCc3ccccc3)nc2OCc2ccccc2)c2ccc([C@H]3CCN(C(=O)OC(C)(C)C)C[C@@H]3F)cc21 ; Cn1c(=O)n(-c2ccc(OCc3ccccc3)nc2OCc2ccccc2)c2ccc([C@@H]3CCN(C(=O)OC(C)(C)C)C[C@H]3F)cc21]

Separation method for Diastercomers: 1.4 g of tert-butyl 4-[1-(2,6-dibenzyloxy-3-pyridyl)-3-methyl-2-oxo-benzimidazol-5-yl]-3-fluoro-piperidine-1-carboxylate (1) was subjected to chiral SFC. Method: Column Name: Chiralpak OX-H; FlowRate: 5 mL/min, Co-Solvent: 35%, Co-Solvent Name: 0.5% Isopropyl Amine in IPA; Outlet Pressure: 100 bar; Injected Volume: 10 μl, Temperature: 40° C., to give tert-butyl (3R,4R)-4-[1-(2,6-dibenzyloxy-3-pyridyl)-3-methyl-2-oxo-benzimidazol-5-yl]-3-fluoro-piperidine-1-carboxylate (B-397, 520 mg, 804.68 μmol, fast eluting fraction) as an off-white solid and tert-butyl (3S,4S)-4-[1-(2,6-dibenzyloxy-3-pyridyl)-3-methyl-2-oxo-benzimidazol-5-yl]-3-fluoro-piperidine-1-carboxylate (B-398, 480 mg, 743.83 μmol, late eluting fraction) as an off-white solid.

### 3-[5-(4-fluoro-3-piperidyl)-3-methyl-2-oxo-benzimidazol-1-yl]piperidine-2,6-dione (B-399)

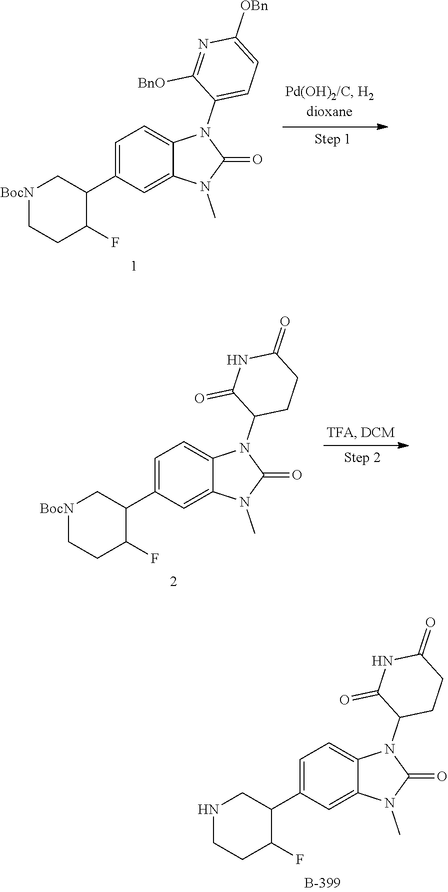 [SMILES: Cn1c(=O)n(-c2ccc(OCc3ccccc3)nc2OCc2ccccc2)c2ccc(C3CN(C(=O)OC(C)(C)C)CCC3F)cc21 ; Cn1c(=O)n(C2CCC(=O)NC2=O)c2ccc(C3CN(C(=O)OC(C)(C)C)CCC3F)cc21 ; Cn1c(=O)n(C2CCC(=O)NC2=O)c2ccc(C3CNCCC3F)cc21]

### Step 1: tert-butyl 3-[1-(2,6-dioxo-3-piperidyl)-3-methyl-2-oxo-benzimidazol-5-yl]-4-fluoro-piperidine-1-carboxylate (2)

tert-butyl 3-[1-(2,6-dioxo-3-piperidyl)-3-methyl-2-oxo-benzimidazol-5-yl]-4-fluoro-piperidine-1-carboxy late (2, 450 mg, 967.43 μmol, 88% yield) was synthesized from tert-butyl 3-[1-(2,6-dibenzyloxy-3-pyridyl)-3-methyl-2-oxo-benzimidazol-5-yl]-4-fluoro-piperidine-1-carboxylate (1, 700 mg, 1.10 mmol) in a similar fashion to Compound B-103, except using 20 wt. % palladium hydroxide on carbon, 50% water (461.74 mg, 657.56 μmol). LCMS (ES+): m/z 405.2 [M−tBu+H]⁺.

### Step 2: 3-[5-(4-fluoro-3-piperidyl)-3-methyl-2-oxo-benzimidazol-1-yl]piperidine-2,6-dione (B-399)

3-[5-(4-fluoro-3-piperidyl)-3-methyl-2-oxo-benzimidazol-1-yl]piperidine-2,6-dione (B-399, 455 mg, 957.47 μmol, 99% yield, TFA salt) was synthesized from tert-butyl 3-[1-(2,6-dioxo-3-piperidyl)-3-methyl-2-oxo-benzimidazol-5-yl]-4-fluoro-piperidine-1-carboxy late (2, 450 mg, 967.43 μmol) in a similar fashion to Compound A-62, except using 20 eq. TFA. The material was purified by reverse phase prep-HPLC (Purification method: Column: X-Select C18 (150×19) mm 5 micron, mobile phase: 0.1% TFA in water and MeCN). LCMS (ES+): m/z 361.1 [M+H]⁺.

### tert-butyl (3R,4R)-3-[1-(2,6-dibenzyloxy-3-pyridyl)-3-methyl-2-oxo-benzimidazol-5-yl]-4-fluoro-piperidine-1-carboxylate (B-400a) and tert-butyl (3S,4S)-3-[1-(2,6-dibenzyloxy-3-pyridyl)-3-methyl-2-oxo-benzimidazol-5-yl]-4-fluoro-piperidine-1-carboxylate (B-400b)

Configurations are arbitrarily assigned.

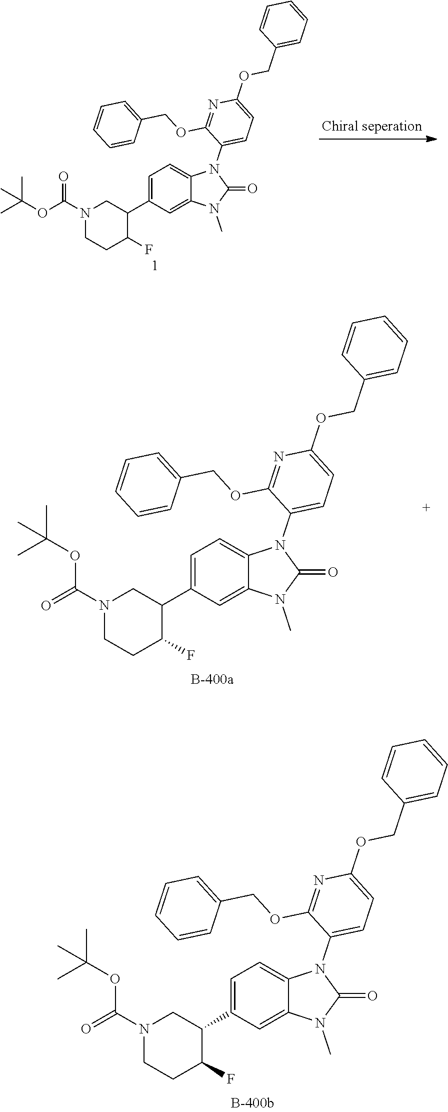 [SMILES: Cn1c(=O)n(-c2ccc(OCc3ccccc3)nc2OCc2ccccc2)c2ccc(C3CN(C(=O)OC(C)(C)C)CCC3F)cc21 ; Cn1c(=O)n(-c2ccc(OCc3ccccc3)nc2OCc2ccccc2)c2ccc(C3CN(C(=O)OC(C)(C)C)CC[C@H]3F)cc21 ; Cn1c(=O)n(-c2ccc(OCc3ccccc3)nc2OCc2ccccc2)c2ccc([C@H]3CN(C(=O)OC(C)(C)C)CC[C@@H]3F)cc21]

1.4 g of tert-butyl 3-[1-(2,6-dibenzyloxy-3-pyridyl)-3-methyl-2-oxo-benzimidazol-5-yl]-4-fluoro-piperidine-1-carboxylate (1) was subjected to chiral SFC. Method: Column Name: Lux A1, Flow rate: 4 mL/min, Co-Solvent Name: 0.5% Isopropyl Amine in Methanol; Outlet Pressure: 100 bar, Injected Volume: 2 μl, Temperature: 35° C.

Diastercomer 1: tert-butyl (3R,4R)-3-[1-(2,6-dibenzyloxy-3-pyridyl)-3-methyl-2-oxo-benzimidazol-5-yl]-4-fluoro-piperidine-1-carboxylate (B-400a, 575 mg, 891.23 μmol, fast eluting fraction) as an off-white solid.

Diastereomer 2: tert-butyl (3S,4S)-3-[1-(2,6-dibenzyloxy-3-pyridyl)-3-methyl-2-oxo-benzimidazol-5-yl]-4-fluoro-piperidine-1-carboxy late (B-400b, 500 mg, late eluting fraction) as an off-white solid.

### tert-butyl N-[2-[1-[1-(2,6-dioxo-3-piperidyl)-3-methyl-2-oxo-benzimidazol-5-yl]-4-piperidyl]ethyl]carbamate (B-401)

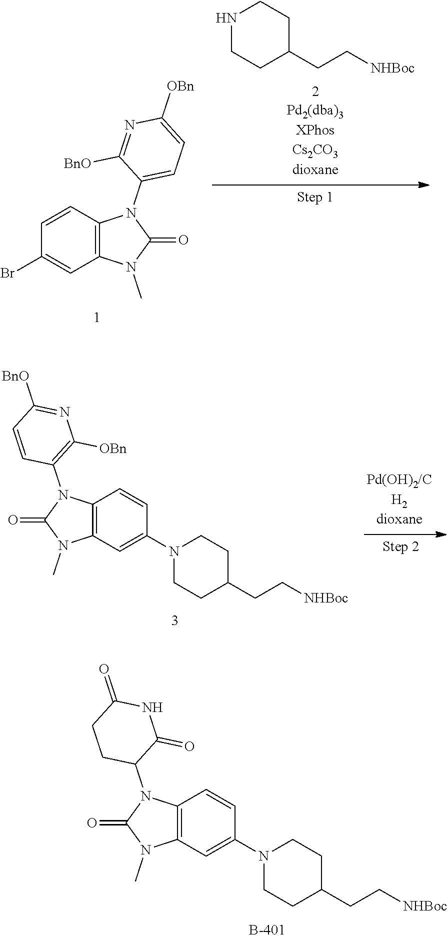 [SMILES: CC(C)(C)OC(=O)NCCC1CCNCC1 ; Cn1c(=O)n(-c2ccc(OCc3ccccc3)nc2OCc2ccccc2)c2ccc(Br)cc21 ; Cn1c(=O)n(-c2ccc(OCc3ccccc3)nc2OCc2ccccc2)c2ccc(N3CCC(CCNC(=O)OC(C)(C)C)CC3)cc21 ; Cn1c(=O)n(C2CCC(=O)NC2=O)c2ccc(N3CCC(CCNC(=O)OC(C)(C)C)CC3)cc21]

### Step 1: tert-butyl N-[2-[1-[1-(2,6-dibenzyloxy-3-pyridyl)-3-methyl-2-oxo-benzimidazol-5-yl]-4-piperidyl]ethyl]carbamate (3)

To a solution of tert-butyl N-[2-(4-piperidyl)ethyl]carbamate (2, 1.39 g, 6.10 mmol) and 5-bromo-1-(2,6-dibenzyloxy-3-pyridyl)-3-methyl-benzimidazol-2-one (1, 3 g, 5.81 mmol) in dioxane (30 mL) was added cesium carbonate (3.79 g, 11.62 mmol), dicyclohexyl-[2-(2,4,6-triisopropylphenyl)phenyl]phosphane (553.91 mg, 1.16 mmol) and chloroform; (1E,4E)-1,5-diphenylpenta-1,4-dien-3-one; palladium (601.35 mg, 580.96 μmol). The mixture was stirred at 90° C. for 16 h under N2. The residue was purified by column chromatography (SiO₂, Petroleum ether/Ethyl acetate=1:1) to give tert-butyl N-[2-[1-[1-(2,6-dibenzyloxy-3-pyridyl)-3-methyl-2-oxo-benzimidazol-5-yl]-4-piperidyl]ethyl]carbamate (3, 3.2 g, 4.68 mmol, 80% yield) as a yellow solid. LCMS (ES+): m/z 664.2 [M+H]⁺.

### Step 2: tert-butyl N-[2-[1-[1-(2,6-dioxo-3-piperidyl)-3-methyl-2-oxo-benzimidazol-5-yl]-4-piperidyl]ethyl]carbamate (B-401)

tert-butyl N-[2-[1-[1-(2,6-dioxo-3-piperidyl)-3-methyl-2-oxo-benzimidazol-5-yl]-4-piperidyl]ethyl]carbamate (B-401, 500 mg, 938.06 μmol, 90% yield) was synthesized from tert-butyl N-[2-[1-[1-(2,6-dibenzyloxy-3-pyridyl)-3-methyl-2-oxo-benzimidazol-5-yl]-4-piperidyl]ethyl]carbamate (3, 700 mg, 1.05 mmol) in a similar fashion to Compound B-103, except using 20 wt. % palladium hydroxide on carbon, 50% water (587.63 mg, 4.18 mmol) and triturating the product with diethyl ether. LCMS (ES+): m/z 486.2 [M+H]⁺.

### 3-[5-[4-(2-aminoethyl)-1-piperidyl]-3-methyl-2-oxo-benzimidazol-1-yl]piperidine-2,6-dione (B-402)

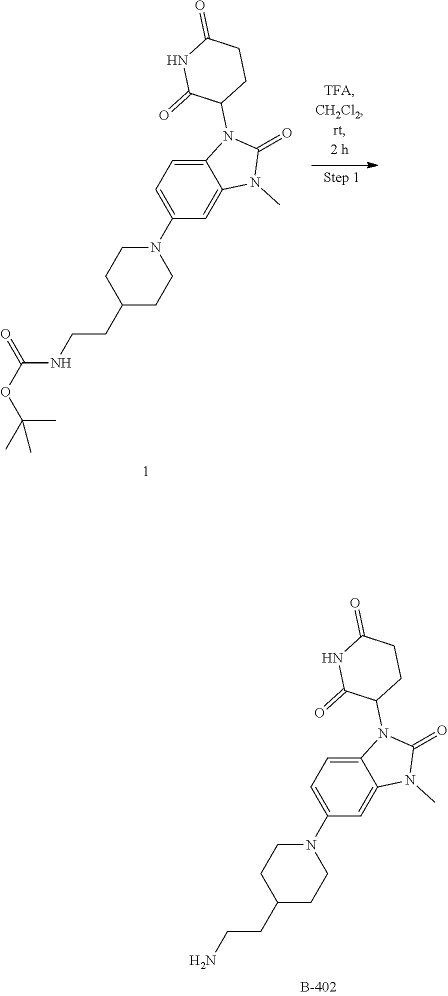 [SMILES: Cn1c(=O)n(C2CCC(=O)NC2=O)c2ccc(N3CCC(CCNC(=O)OC(C)(C)C)CC3)cc21 ; Cn1c(=O)n(C2CCC(=O)NC2=O)c2ccc(N3CCC(CCN)CC3)cc21]

### Step 1: 3-[5-[4-(2-aminoethyl)-1-piperidyl]-3-methyl-2-oxo-benzimidazol-1-yl]piperidine-2,6-dione (B-402)

3-[5-[4-(2-aminoethyl)-1-piperidyl]-3-methyl-2-oxo-benzimidazol-1-yl]piperidine-2,6-dione (B-402, 460 mg, 912.39 μmol, 89% yield, TFA salt) was synthesized from tert-butyl N-[2-[1-[1-(2,6-dioxo-3-piperidyl)-3-methyl-2-oxo-benzimidazol-5-yl]-4-piperidyl]ethyl]carbamate (1, 500 mg, 1.03 mmol) in a similar fashion to Compound A-62, except using 32 eq. TFA. LCMS (ES+): m/z 386.1 [M+H]⁺.

### 3-[5-(3,3-difluoro-4-piperidyl)-3-isopropyl-2-oxo-benzimidazol-1-yl]piperidine-2,6-dione (B-403)

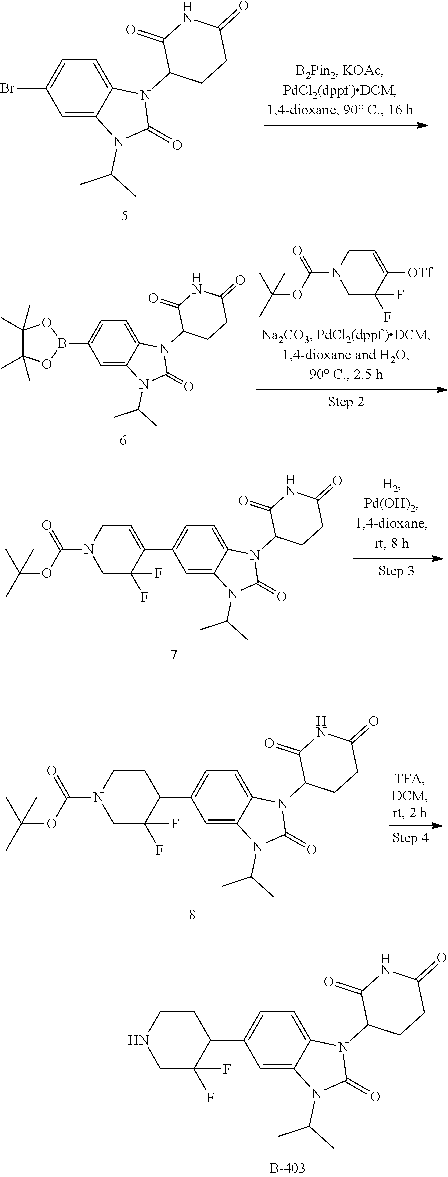

### Step 1: 3-[3-isopropyl-2-oxo-5-(4,4,5,5-tetramethyl-1,3,2-dioxaborolan-2-yl)benzimidazol-1-yl]piperidine-2,6-dione (6)

Into a 50 mL pressure tube containing a well-stirred solution of 3-(5-bromo-3-isopropyl-2-oxo-benzimidazol-1-yl)piperidine-2,6-dione (5, 680 mg, 1.85 mmol) in 1,4-dioxane (8 mL) was added bis(pinacolato)diboron (515.83 mg, 2.03 mmol) and potassium acetate (543.70 mg, 5.54 mmol). The reaction mixture was degassed by bubbling nitrogen gas through for 10 min. Then, 1,1′-bis(diphenylphosphino)ferrocene]dichloropalladium(II), complex with dichloromethane (150.80 mg, 184.66 μmol) was added and the reaction mixture was heated at 90° C. After 16 h, the reaction mixture was filtered through Celite and washed with ethyl acetate (200 mL). The filtrate was washed with water (100 mL), brine solution (50 mL) and dried over Na₂SO₄, filtered and purified by flash silica gel column chromatography (10-95% pet ether in ethyl acetate) to afford 3-[3-isopropyl-2-oxo-5-(4,4,5,5-tetramethyl-1,3,2-dioxaborolan-2-yl)benzimidazol-1-yl]piperidine-2,6-dione (6, 600 mg, 1.09 mmol, 59% yield, 75% purity) as an off-white solid.

LCMS (ES+): m/z 414.2 [M+H]⁺.

### Step 2: tert-butyl 4-[1-(2,6-dioxo-3-piperidyl)-3-isopropyl-2-oxo-benzimidazol-5-yl]-3,3-difluoro-2,6-dihydropyridine-1-carboxylate (7)

Into a 50 mL pressure tube containing a well-stirred solution of 3-[3-isopropyl-2-oxo-5-(4,4,5,5-tetramethyl-1,3,2-dioxaborolan-2-yl)benzimidazol-1-yl]piperidine-2,6-dione (6, 600 mg, 1.09 mmol) in 1,4-dioxane (6 mL) and water (0.2 mL) was added tert-butyl 3,3-difluoro-4-(trifluoromethylsulfonyloxy)-2,6-dihydropyridine-1-carboxylate (784.17 mg, 1.09 mmol) and sodium carbonate (115.41 mg, 1.09 mmol). The reaction mixture was degassed by bubbling nitrogen gas through for 10 min. Then, Pd(dppf)Cl₂·DCM (88.92 mg, 108.89 μmol) was added and the reaction mixture was heated at 90° C. After 2.5 h, the reaction mixture was filtered through Celite and washed with ethyl acetate (150 mL). The filtrate was washed with water followed (150 mL) and brine solution (100 mL) and dried over Na₂SO₄, filtered and purified by flash silica gel column chromatography (5-75% of ethyl acetate in pet-ether) to afford tert-butyl 4-[1-(2,6-dioxo-3-piperidyl)-3-isopropyl-2-oxo-benzimidazol-5-yl]-3,3-difluoro-2,6-dihydropyridine-1-carboxylate (7, 630 mg, 949.01 μmol, 87% yield, 76% purity) as a brown solid. LCMS (ES+): m/z 505.3 [M+H]⁺.

### Step 3: ter-butyl 4-[1-(2,6-dioxo-3-piperidyl)-3-isopropyl-2-oxo-benzimidazol-5-yl]-3,3-difluoro-piperidine-1-carboxylate (8)

tert-butyl 4-[1-(2,6-dioxo-3-piperidyl)-3-isopropyl-2-oxo-benzimidazol-5-yl]-3,3-difluoro-piperidine-1-carboxylate (8, 530 mg, 941.68 μmol, 99% yield) was synthesized from tert-butyl 4-[1-(2,6-dioxo-3-piperidyl)-3-isopropyl-2-oxo-benzimidazol-5-yl]-3,3-difluoro-2,6-dihydropyridine-1-carboxylate (7, 630 mg, 949.01 μmol) in a similar fashion to Compound B-103, except using 20 wt. % palladium hydroxide on carbon (399.81 mg, 2.85 mmol). LCMS (ES+): m/z 507.2 [M+H]⁺.

### Step 4: 3-[5-(3,3-difluoro-4-piperidyl)-3-isopropyl-2-oxo-benzimidazol-1-yl]piperidine-2,6-dione (B-403)

3-[5-(3,3-difluoro-4-piperidyl)-3-isopropyl-2-oxo-benzimidazol-1-yl]piperidine-2,6-dione (B-403, 513 mg, 977.80 μmol, 93% yield, TFA salt) was synthesized from tert-butyl 4-[1-(2,6-dioxo-3-piperidyl)-3-isopropyl-2-oxo-benzimidazol-5-yl]-3,3-difluoro-piperidine-1-carboxylate (8, 530 mg, 1.05 mmol) in a similar fashion to Compound A-62, except using 20 eq. TFA and triturating with diethyl ether. LCMS (ES+): m/z 407.1 [M+H]⁺.

### 2-[4-[1-(2,6-dioxo-3-piperidyl)-3-isopropyl-2-oxo-benzimidazol-5-yl]-3,3-difluoro-1-piperidyl]acetic acid (B-404)

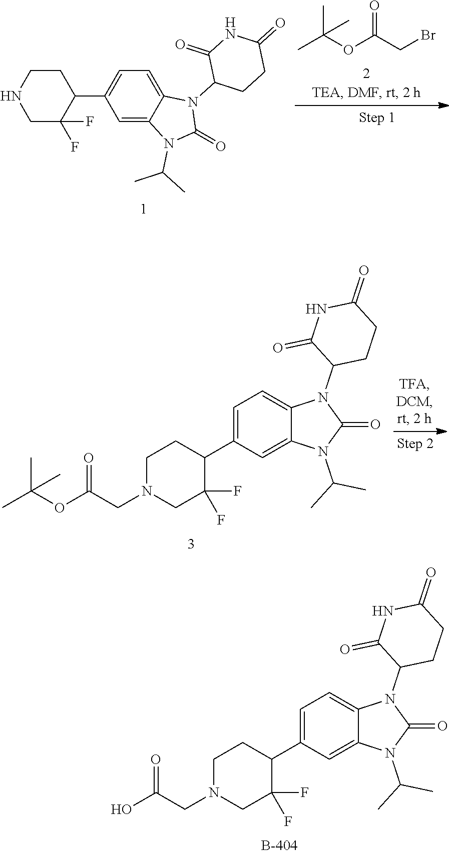

### Step 1: tert-butyl 2-[4-[1-(2,6-dioxo-3-piperidyl)-3-isopropyl-2-oxo-benzimidazol-5-yl]-3,3-difluoro-1-piperidyl]acetate (3)

Into a 20 mL vial containing a well-stirred solution of 3-[5-(3,3-difluoro-4-piperidyl)-3-isopropyl-2-oxo-benzimidazol-1-yl]piperidine-2,6-dione (1, 230 mg, 362.38 μmol, TFA salt) in DMF (2 mL) was added triethylamine (183.35 mg, 1.81 mmol, 252.54 μL) and tert-butyl 2-bromoacetate (2, 84.82 mg, 434.86 μmol). After 2 h, the reaction mixture was concentrated under vacuum and the residue was suspended in water (5 mL). The solid was filtered and washed with water and diethyl ether, and dried under vacuum to afford tert-butyl 2-[4-[1-(2,6-dioxo-3-piperidyl)-3-isopropyl-2-oxo-benzimidazol-5-yl]-3,3-difluoro-1-piperidyl]acetate (3, 80 mg, 129.09 μmol, 36% yield) as a brown solid.

LCMS (ES+): m/z 521.2 [M+H]⁺.

### Step 2: 2-[4-[1-(2,6-dioxo-3-piperidyl)-3-isopropyl-2-oxo-benzimidazol-5-yl]-3,3-difluoro-1-piperidyl]acetic acid (B-404)

2-[4-[1-(2,6-dioxo-3-piperidyl)-3-isopropyl-2-oxo-benzimidazol-5-yl]-3,3-difluoro-1-piperidyl]acetic acid. (B-404, 70 mg, 104.07 μmol, 81% yield, TFA salt) was synthesized from tert-butyl 2-[4-[1-(2,6-dioxo-3-piperidyl)-3-isopropyl-2-oxo-benzimidazol-5-yl]-3,3-difluoro-1-piperidyl]acetate (3, 80 mg, 129.09 μmol) in a similar fashion to Compound A-26, except using 20 eq. TFA.

LCMS (ES+): m/z 465.2 [M+H]⁺.

### 3-[3-methyl-2-oxo-5-(4-oxo-1-piperidyl)benzimidazol-1-yl]piperidine-2,6-dione (B-405)

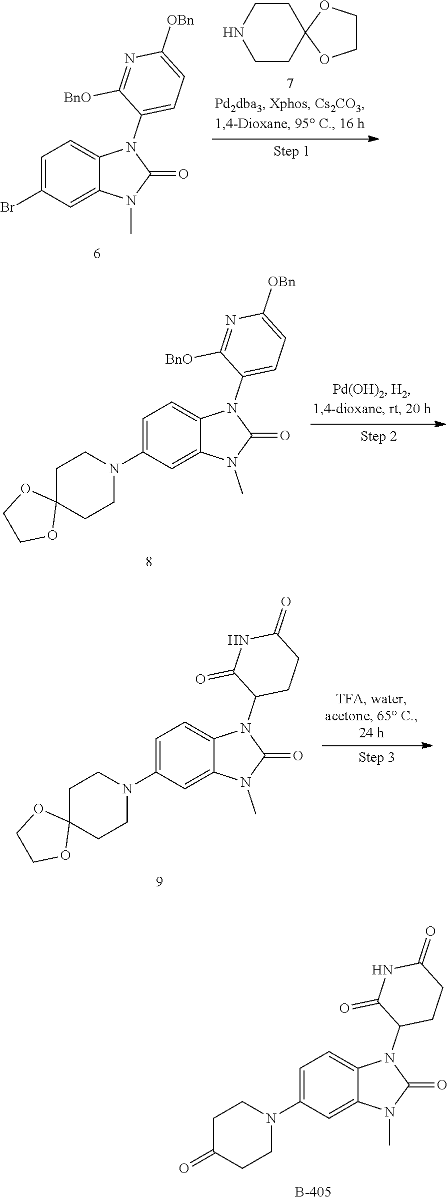 [SMILES: Cn1c(=O)n(-c2ccc(OCc3ccccc3)nc2OCc2ccccc2)c2ccc(Br)cc21 ; C1CC2(CCN1)OCCO2 ; Cn1c(=O)n(-c2ccc(OCc3ccccc3)nc2OCc2ccccc2)c2ccc(N3CCC4(CC3)OCCO4)cc21 ; Cn1c(=O)n(C2CCC(=O)NC2=O)c2ccc(N3CCC4(CC3)OCCO4)cc21 ; Cn1c(=O)n(C2CCC(=O)NC2=O)c2ccc(N3CCC(=O)CC3)cc21]

### Step 1: 1-(2,6-dibenzyloxy-3-pyridyl)-5-(1,4-dioxa-8-azaspiro[4.5]decan-8-yl)-3-methyl-benzimidazol-2-one (8)

1-(2,6-dibenzyloxy-3-pyridyl)-5-(1,4-dioxa-8-azaspiro[4.5]decan-8-yl)-3-methyl-benzimidazol-2-one (8, 1.4 g, 2.37 mmol, 65% yield) was synthesized from 5-bromo-1-(2,6-dibenzyloxy-3-pyridyl)-3-methyl-benzimidazol-2-one (6, 2 g, 3.64 mmol) and 1,4-dioxa-8-azaspiro[4.5]decane (7, 1.56 g, 10.92 mmol) in a similar fashion to Compound B-180, except 0.1 eq. Pd₂dba3, 0.15 eq. X-phos and heating at 95° C.

LCMS (ES+): m/z 579.2 [M+H]⁺.

### Step 2: 3-[5-(1,4-dioxa-8-azaspiro[4.5]decan-8-yl)-3-methyl-2-oxo-benzimidazol-1-yl]piperidine-2,6-dione (9)

3-[5-(1,4-dioxa-8-azaspiro[4.5]decan-8-yl)-3-methyl-2-oxo-benzimidazol-1-yl]piperidine-2,6-dione (9, 0.8 g, 1.99 mmol, 84% yield) was synthesized from 1-(2,6-dibenzyloxy-3-pyridyl)-5-(1,4-dioxa-8-azaspiro[4.5]decan-8-yl)-3-methyl-benzimidazol-2-one (8, 1.4 g, 2.37 mmol) in a similar fashion to Compound B-103, except using 20 wt. % palladium hydroxide on carbon (1 g, 1.42 mmol). LCMS (ES+): m/z 401.2 [M+H]⁺.

### Step 3: 3-[3-methyl-2-oxo-5-(4-oxo-1-piperidyl)benzimidazol-1-yl]piperidine-2,6-dione (B-405)

Into a 50 mL pressure tube containing a well-stirred solution of 3-[5-(1,4-dioxa-8-azaspiro[4.5]decan-8-yl)-3-methyl-2-oxo-benzimidazol-1-yl]piperidine-2,6-dione (9, 0.8 g, 1.98 mmol) in acetone (4 mL) and water (3 mL) was added TFA (8.88 g, 77.88 mmol, 6 mL) at rt. The reaction mixture was stirred at 65° C. After 16 h, the solvent was removed under reduced pressure and the residue was washed with diethyl ether (30 mL) to afford 3-[3-methyl-2-oxo-5-(4-oxo-1-piperidyl)benzimidazol-1-yl]piperidine-2,6-dione (B-405, 0.7 g, 401.79 μmol, 20% yield, 27% purity, TFA salt) as an off-white solid. The material was taken to next step without purification.

LCMS (ES+): m/z 357.1 [M+H]⁺.

### 2-(4-(1-(2,6-dioxopiperidin-3-yl)-3-methyl-2-oxo-2,3-dihydro-1H-benzo[d]imidazol-5-yl)-3-fluorophenyl)acetic acid (B-406)

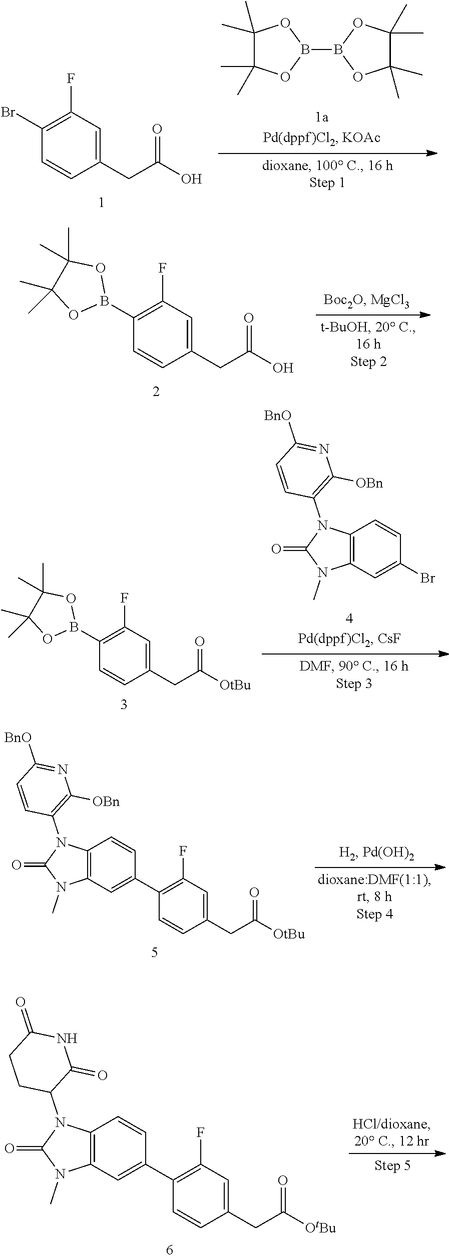

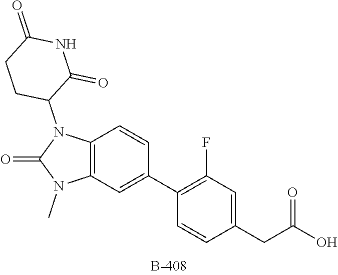 [SMILES: Cn1c(=O)n(C2CCC(=O)NC2=O)c2ccc(-c3ccc(CC(=O)O)cc3F)cc21]

### Step 1: 2-(3-fluoro-4-(4,4,5,5-tetramethyl-1,3,2-dioxaborolan-2-yl)phenyl)acetic acid (2)

To a solution of 2-(4-bromo-3-fluoro-phenyl)acetic acid (1, 2 g, 8.58 mmol) and Bis(pinacolato)diboron (1a, 3.27 g, 12.87 mmol) in dioxane (5 mL) was added KOAc (4.21 g, 42.91 mmol) under N₂ atmosphere. Subsequently, Pd(dppf)Cl₂ (313.99 mg, 429.12 μmol) was added to the reaction mixture and reaction mixture was stirred at 100° C. for 16 h. The reaction mixture diluted with water (10 mL) and extracted with ethyl acetate (20 mL×3). The organic phases were combined and washed with brine (30 mL), dried over anhydrous Na₂SO₄, filtered and concentrated under reduced pressure. The residue was purified by column chromatography (SiO₂, petroleum ether/ethyl acetate=100/0 to 1/1) to afford 2-(3-fluoro-4-(4,4,5,5-tetramethyl-1,3,2-dioxaborolan-2-yl)phenyl)acetic acid (2, 1.7 g, 6.07 mmol, 71% yield).

¹H NMR (400 MHz, CDCl₃) δ 7.71 (dd, J=6.4, 7.2 Hz, 1H), 7.07 (dd, J=1.2, 7.6 Hz, 1H), 7.01-6.97 (m, 1H), 3.65 (s, 2H), 1.36 (s, 12H).

### Step 2: tert-butyl 2-(3-fluoro-4-(4,4,5,5-tetramethyl-1,3,2-dioxaborolan-2-yl)phenyl)acetate (3)

To a mixture of 2-(3-fluoro-4-(4,4,5,5-tetramethyl-1,3,2-dioxaborolan-2-yl)phenyl)acetic acid (2, 1.7 g, 6.07 mmol) and Boc₂O (1.72 g, 7.89 mmol, 1.81 mL) in tert-butyl alcohol (10 mL) was added MgCl₂ (57.79 mg, 606.93 μmol) and stirred for 16 h. The reaction mixture was diluted with water (10 mL) and extracted with ethyl acetate (20 mL×3). The organic phases were combined and washed with brine (30 mL), dried over anhydrous Na₂SO₄, filtered and concentrated under reduced pressure. The residue was purified by column chromatography (SiO₂, petroleum ether/ethyl acetate=100/0 to 5/1) to afford tert-butyl 2-(3-fluoro-4-(4,4,5,5-tetramethyl-1,3,2-dioxaborolan-2-yl)phenyl)acetate (3, 1.3 g, 3.87 mmol, 64% yield).

¹H NMR (400 MHz, CDCl₃) δ 7.67 (dd, J=6.4, 7.2 Hz, 1H), 7.04 (dd, J=1.2, 7.6 Hz, 1H), 6.97 (dd, J=1.2, 10.0 Hz, 1H), 3.51 (s, 2H), 1.42 (s, 9H), 1.35 (s, 12H).

### Step 3: tert-butyl 2-(4-(1-(2,6-bis(benzyloxy)pyridin-3-yl)-3-methyl-2-oxo-2,3-dihydro-1H-benzo[d]imidazol-5-yl)-3-fluorophenyl)acetate (5)

tert-butyl 2-(4-(1-(2,6-bis(benzyloxy)pyridin-3-yl)-3-methyl-2-oxo-2,3-dihydro-1H-benzo[d]imidazol-5-yl)-3-fluorophenyl)acetate (5, 1.5 g, 2.11 mmol, 77% yield) was synthesized from tert-butyl 2-(3-fluoro-4-(4,4,5,5-tetramethyl-1,3,2-dioxaborolan-2-yl)phenyl)acetate (3, 1.2 g, 3.57 mmol) and 1-(2,6-bis(benzyloxy)pyridin-3-yl)-5-bromo-3-methyl-1H-benzo[d]imidazol-2(3H)-one (4, 1.42 g, 2.75 mmol) in a similar fashion to Compound B-133, except using 0.05 eq. Pd(dppf)Cl₂. LCMS (ESI): m/z 646.2 [M+H]⁺. ¹H NMR (400 MHz, d6-DMSO) δ 7.86-7.83 (m, 1H), 7.51-7.44 (m, 3H), 7.40-7.36 (m, 3H), 7.29-7.25 (m, 6H), 7.20-7.17 (m, 2H), 6.78 (d, J=8.2 Hz, 1H), 6.65-6.61 (m, 1H), 5.42-5.37 (m, 4H), 3.93 (s, 1H), 3.65 (s, 2H), 3.43 (s, 3H), 1.43 (s, 9H).

### Step 4: tert-butyl 2-(4-(1-(2,6-dioxopiperidin-3-yl)-3-methyl-2-oxo-2,3-dihydro-1H-benzo[d]imidazol-5-yl)-3-fluorophenyl)acetate (6)

tert-butyl 2-(4-(1-(2,6-dioxopiperidin-3-yl)-3-methyl-2-oxo-2,3-dihydro-1H-benzo[d]imidazol-5-yl)-3-fluorophenyl)acetate (6, 9.55 mg, 20.22 μmol, 26% yield) was synthesized from tert-butyl 2-(4-(1-(2,6-bis(benzyloxy)pyridin-3-yl)-3-methyl-2-oxo-2,3-dihydro-1H-benzo[d]imidazol-5-yl)-3-fluorophenyl)acetate (5, 50 mg, 77.43 μmol) in a similar fashion to Compound B-103, except using Pd(OH)₂/C (17.07 mg). The material was purified by reverse phase prep-HPLC (flow: 25 mL/min; gradient: from 25-55% MeCN in water (0.1% TFA); column: Phenomenex Synergi C18 150×25 mm×10 μm), LCMS (ESI): m/z 468.2 [M+H]⁺. ¹H NMR (400 MHz, d₆-DMSO) δ 11.13 (s, 1H), 7.50 (t, J=8.4 Hz, 1H), 7.36 (s, 1H), 7.24-7.15 (m, 4H), 5.41 (dd, J=5.2, 12.8 Hz, 1H), 3.64 (s, 2H), 3.38-3.38 (m, 3H), 2.98-2.86 (m, 1H), 2.81-2.71 (m, 1H), 2.68-2.60 (m, 1H), 2.10-1.99 (m, 1H), 1.42 (s, 9H).

### Step 5: 2-(4-(1-(2,6-dioxopiperidin-3-yl)-3-methyl-2-oxo-2,3-dihydro-1H-benzo[d]imidazol-5-yl)-3-fluorophenyl)acetic acid (B-406)

2-(4-(1-(2,6-dioxopiperidin-3-yl)-3-methyl-2-oxo-2,3-dihydro-1H-benzo[d]imidazol-5-yl)-3-fluorophenyl)acetic acid (B-406, 77 mg, 145.09 μmol, 48% yield, TFA salt) was synthesized from tert-butyl 2-(4-(1-(2,6-dioxopiperidin-3-yl)-3-methyl-2-oxo-2,3-dihydro-1H-benzo[d]imidazol-5-yl)-3-fluorophenyl)acetate (6, 140 mg, 299.47 μmol) in a similar fashion to—(6, step 3 of Compound A-148/Compound A-149, except using 40 eq. HCl/dioxane (4 M) and stirring at rt. The product was purified by reversed flash (flow: 30 mL/min; gradient: from 10-30% MeCN in water (0.1% TFA) over 10 min; column: Welch Ultimate XB—C18, 20-40 μm, 100 Å, I.D.95 mm×H 365 mm). LCMS (ESI): m/z 412.2 [M+H]⁺.

### 3-[6-(3,3-difluoro-4-piperidyl)-2-oxo-benzo[cd]indol-1-yl]piperidine-2,6-dione (B-407)

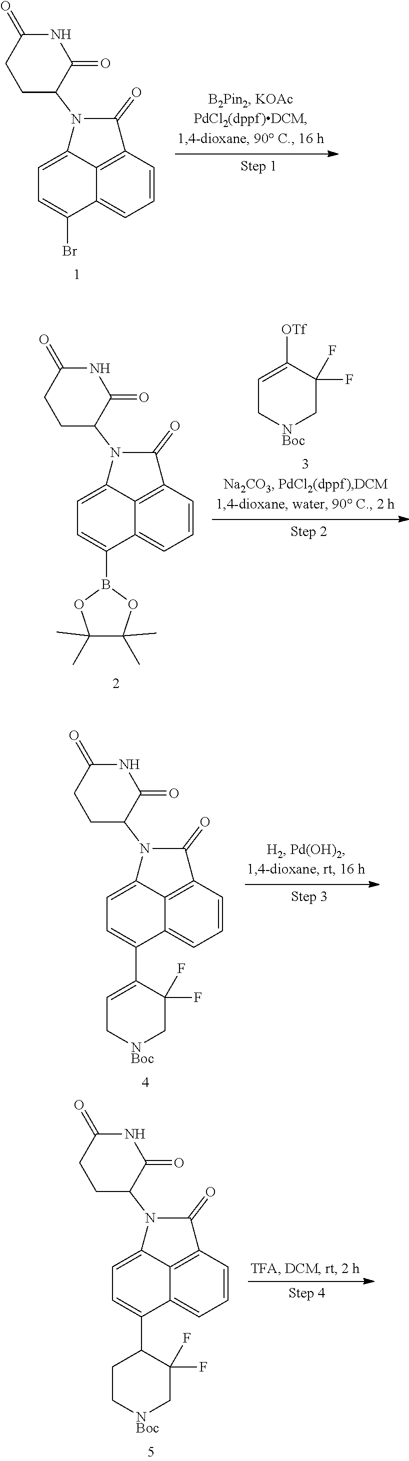 [SMILES: O=C1CCC(N2C(=O)c3cccc4c(Br)ccc2c34)C(=O)N1 ; CC1(C)OB(c2ccc3c4c(cccc24)C(=O)N3C2CCC(=O)NC2=O)OC1(C)C ; CC(C)(C)OC(=O)N1CC=C(OS(=O)(=O)C(F)(F)F)C(F)(F)C1 ; CC(C)(C)OC(=O)N1CC=C(c2ccc3c4c(cccc24)C(=O)N3C2CCC(=O)NC2=O)C(F)(F)C1 ; CC(C)(C)OC(=O)N1CCC(c2ccc3c4c(cccc24)C(=O)N3C2CCC(=O)NC2=O)C(F)(F)C1]

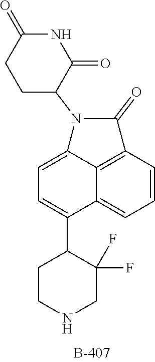 [SMILES: O=C1CCC(N2C(=O)c3cccc4c(C5CCNCC5(F)F)ccc2c34)C(=O)N1]

### Step 1: 3-[2-oxo-6-(4,4,5,5-tetramethyl-1,3,2-dioxaborolan-2-yl)benzo[cd]indol-1-yl]piperidine-2,6-dione (2)

Into a 25 mL pressure tube containing a well-stirred solution of 3-(6-bromo-2-oxo-benzo[cd]indol-1-yl)piperidine-2,6-dione (1, 500 mg, 1.27 mmol) in 1,4-dioxane (10 mL) was added bis(pinacolato)diboron (353.86 mg, 1.39 mmol) and potassium acetate (372.97 mg, 3.80 mmol, 237.56 μL). The suspension was degassed by bubbling of nitrogen gas for 5 min. Then, 1,1′-bis(diphenylphosphino)ferrocene-palladium(II)dichloride dichloromethane complex (103.40 mg, 126.68 μmol) was added and degassed for another 5 min. The tube was sealed, and reaction mixture was heated to 90° C. After 16 h, the reaction mixture was filtered through Celite and washed with ethyl acetate (100 mL). The filtrate was concentrated under reduced pressure. The residue was purified by column chromatography (40-50% EtOAc in pet ether) to afford 3-[2-oxo-6-(4,4,5,5-tetramethyl-1,3,2-dioxaborolan-2-yl)benzo[cd]indol-1-yl]piperidine-2,6-dione (2, 430 mg, 726.65 μmol, 57% yield, 69% purity) as a yellow solid.

LCMS (ES+): m/z 407.1 [M+H]⁺.

### Step 2: tert-butyl 4-[1-(2,6-dioxo-3-piperidyl)-2-oxo-benzo[cd]indol-6-yl]-3,3-difluoro-2,6-dihydropyridine-1-carboxylate (4)

Into a 25 mL pressure tube containing a well-stirred solution of 3-[2-oxo-6-(4,4,5,5-tetramethyl-1,3,2-dioxaborolan-2-yl)benzo[cd]indol-1-yl]piperidine-2,6-dione (2, 430 mg, 719.77 μmol) and tert-butyl 3,3-difluoro-4-(trifluoromethylsulfonyloxy)-2,6-dihydropyridine-1-carboxylate (3, 501.38 mg, 791.75 μmol) in anhydrous 1,4-dioxane (2 mL) and water (0.2 mL) was added sodium carbonate (76.29 mg, 719.77 μmol). The mixture was degassed by bubbling nitrogen gas through for 5 min and 1,1′-Bis(diphenylphosphino)ferrocene-palladium(II)dichloride dichloromethane complex (58.75 mg, 71.98 μmol) was added. The tube was sealed and heated at 90° C. After 2 h, the reaction mixture was filtered through Celite, washed with ethyl acetate (150 mL) and concentrated under reduced pressure. The residue was purified by column chromatography (50-60% EtOAc in pet ether) to afford tert-butyl 4-[1-(2,6-dioxo-3-piperidyl)-2-oxo-benzo[cd]indol-6-yl]-3,3-difluoro-2,6-dihydropyridine-1-carboxylate (4, 330 mg, 595.14 μmol, 83% yield) as a pale yellow solid.

LCMS (ES+): m/z 442.1 [M−tBu+H]⁺.

### Step 3: tert-butyl 4-[1-(2,6-dioxo-3-piperidyl)-2-oxo-benzo[cd]indol-6-yl]-3,3-difluoro-piperidine-1-carboxylate (5)

tert-butyl 4-[1-(2,6-dioxo-3-piperidyl)-2-oxo-benzo[cd]indol-6-yl]-3,3-difluoro-piperidine-1-carboxylate (5, 280 mg, 556.13 μmol, 92% yield) was synthesized from tert-butyl 4-[1-(2,6-dioxo-3-piperidyl)-2-oxo-benzo[cd]indol-6-yl]-3,3-difluoro-2,6-dihydropyridine-1-carboxylate (4, 350 mg, 605.04 μmol) in a similar fashion to Compound B-103, except using 20 wt. % palladium hydroxide on carbon (296.89 mg, 423.53 μmol). LCMS (ES+): m/z 440.1 [M−tBu+H]⁻.

### Step 4: 3-[6-(3,3-difluoro-4-piperidyl)-2-oxo-benzo[cd]indol-1-yl]piperidine-2,6-dione (B-407)

3-[6-(3,3-difluoro-4-piperidyl)-2-oxo-benzo[cd]indol-1-yl]piperidine-2,6-dione (B-407, 200 mg, 380.90 μmol, 77% yield, TFA salt) was synthesized from tert-butyl 4-[1-(2,6-dioxo-3-piperidyl)-2-oxo-benzo[cd]indol-6-yl]-3,3-difluoro-piperidine-1-carboxy late (5, 250 mg, 495.49 μmol) in a similar fashion to Compound A-62, except using 10 eq. TFA and triturating with diethyl ether. LCMS (ES+): m/z 400.2 [M+H]⁺.

### tert-butyl 4-[3-(2,6-dioxo-3-piperidyl)-1-methyl-indazol-6-yl]-3,3-difluoro-piperidine-1-carboxylate (B-408)

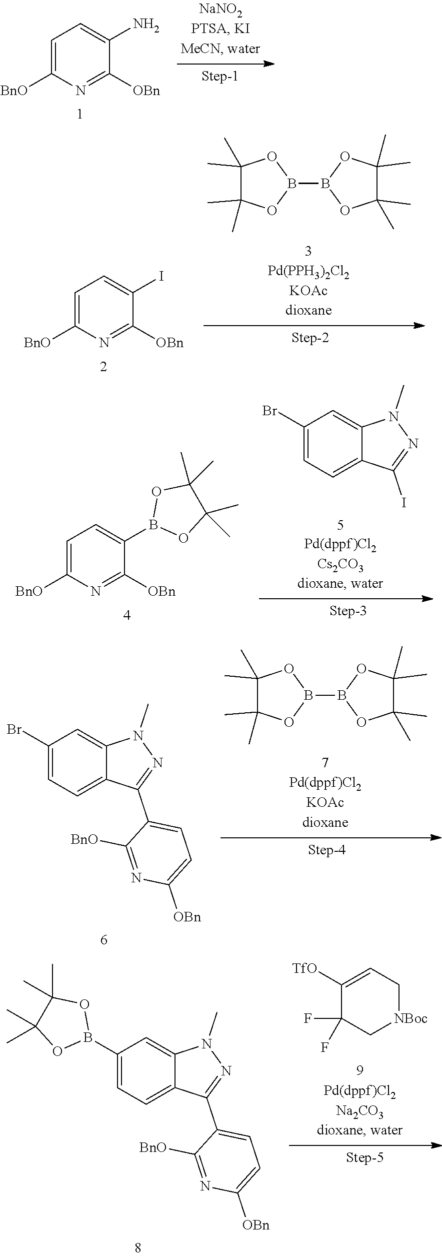

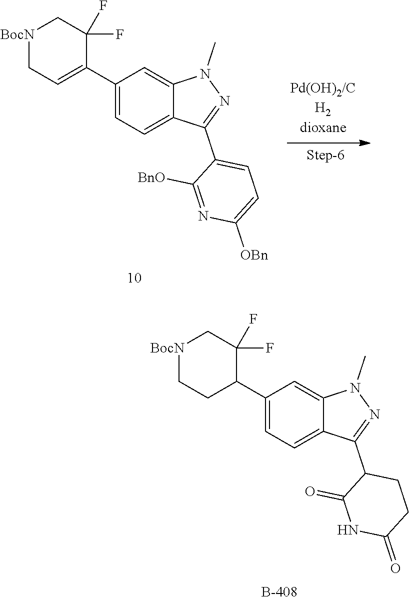 [SMILES: Cn1nc(-c2ccc(OCc3ccccc3)nc2OCc2ccccc2)c2ccc(C3=CCN(C(=O)OC(C)(C)C)CC3(F)F)cc21 ; Cn1nc(C2CCC(=O)NC2=O)c2ccc(C3CCN(C(=O)OC(C)(C)C)CC3(F)F)cc21]

### Step 1: 2,6-dibenzyloxy-3-iodo-pyridine (2)

To a stirred solution of 2,6-dibenzyloxypyridin-3-amine (1, 3 g, 9.79 mmol) in acetonitrile (50 mL) and water (30 mL) at 0° C. were added PTSA (5.06 g, 29.38 mmol) and sodium nitrite (1.35 g, 19.58 mmol). The reaction mixture was stirred at 0° C. for 30 min and potassium iodide (3.41 g, 20.56 mmol) was added before the reaction mixture was warmed up to rt and stirred for 2 h. The reaction was diluted with water (30 mL) and extracted with ethyl acetate (2×100 mL). The combined organic layers were dried over sodium sulfate, filtered, concentrated and purified by column chromatography (silica gel, 10-20% ethyl acetate in pet ether) to afford 2,6-dibenzyloxy-3-iodo-pyridine (2, 2.5 g, 4.19 mmol, 43% yield) as a brown color solid. LCMS (ES⁺): m/z 418.1 [M+H]⁺.

### Step 2: 2,6-dibenzyloxy-3-(4,4,5,5-tetramethyl-1,3,2-dioxaborolan-2-yl)pyridine (4)

In a 500 mL multi-neck round-bottom flask, a solution of 2,6-dibenzyloxy-3-iodo-pyridine (2, 15 g, 35.95 mmol) in 1,4-dioxane (150 mL) was added potassium acetate (10.58 g, 107.85 mmol) and 4,4,5,5-tetramethyl-2-(4,4,5,5-tetramethyl-1,3,2-dioxaborolan-2-yl)-1,3,2-dioxaborolane (3, 27.39 g, 107.85 mmol) at rt. The reaction mixture was purged with nitrogen gas for 10 min and bis(triphenylphosphine) palladium(II)dichloride (2.52 g, 3.60 mmol) was added. The reaction was heated at 100° C. for 16 h. After consumption of the starting material, the reaction mixture was filtered through Celite and washed with ethyl acetate (500 mL). The organic layer was dried over sodium sulfate and concentrated under reduced pressure to afford 2,6-dibenzyloxy-3-(4,4,5,5-tetramethyl-1,3,2-dioxaborolan-2-yl)pyridine (4, 18 g, 19.10 mmol, 53% yield) as a brown gummy, which was directly taken to the next step without any purification. LCMS (ES⁺): m/z 418.2 [M+H]⁺.

### Step 3: 6-bromo-3-(2,6-dibenzyloxy-3-pyridyl)-1-methyl-indazole (6)

In a sealed tube, a solution of 6-bromo-3-iodo-1-methyl-indazole (5, 5 g, 13.95 mmol) and 2,6-dibenzyloxy-3-(4,4,5,5-tetramethyl-1,3,2-dioxaborolan-2-yl)pyridine (4, 12.81 g, 15.34 mmol) in 1,4-dioxane (40 mL) and water (10 mL) was added cesium carbonate (9.09 g, 27.90 mmol) at rt under nitrogen atmosphere. The reaction mixture was degassed with nitrogen for 10 min before Pd(dppf)Cl₂ (1.14 g, 1.39 mmol) was added. The reaction mixture was stirred at 95° C. for 16 h. The reaction was diluted with water (60 mL) and extracted with ethyl acetate (2×100 mL). The combined organic layers were dried over anhydrous sodium sulfate, filtered, and concentrated under reduced pressure. The material was purified by column chromatography (silica gel, 2% ethyl acetate in pet ether) to afford 6-bromo-3-(2,6-dibenzyloxy-3-pyridyl)-1-methyl-indazole (6, 1.25 g, 1.50 mmol, 11% yield) as a light brown gum. LCMS (ES+): m/z 502.2 [M+H]⁺.

### Step 4: 3-(2,6-dibenzyloxy-3-pyridyl)-1-methyl-6-(4,4,5,5-tetramethyl-1,3,2-dioxaborolan-2-yl)indazole (8)

A solution of 6-bromo-3-(2,6-dibenzyloxy-3-pyridyl)-1-methyl-indazole (6, 2 g, 4.00 mmol) in 1,4-dioxane (20 mL), bis(pinacolato)diboron (7, 1.12 g, 4.40 mmol) and potassium acetate (1.18 g, 11.99 mmol, 749.55 μL) was added to a 100 mL sealed tube with stirring. The reaction mixture was degassed with nitrogen for 10 min before Pd(dppf)Cl₂ (326.40 mg, 399.69 μmol) was added, and the reaction degassed again for 5 min. The reaction mixture was stirred at 90° C. for 2 h. The reaction was filtered through Celite, which was washed with ethyl acetate. The organic layer was concentrated and purified by column chromatography (silica gel 230-400 mesh, 20% ethyl acetate in pet ether) to afford 3-(2,6-dibenzyloxy-3-pyridyl)-1-methyl-6-(4,4,5,5-tetramethyl-1,3,2-dioxaborolan-2-yl) indazole (8, 1.9 g, 3.43 mmol, 86% yield) as a gummy yellow liquid. LCMS (ES+): m/z 548.2 (M+H)+.

### Step 5: tert-butyl 4-[3-(2,6-dibenzyloxy-3-pyridyl)-1-methyl-indazol-6-yl]-3,3-difluoro-2,6-dihydropyridine-1-carboxylate (10)

A solution of 3-(2,6-dibenzyloxy-3-pyridyl)-1-methyl-6-(4,4,5,5-tetramethyl-1,3,2-dioxaborolan-2-yl)indazole (8, 500 mg, 913.32 μmol) and tert-butyl 3,3-difluoro-4-(trifluoromethylsulfonyloxy)-2,6-dihydropyridine-1-carboxylate (9, 436.09 mg, 1.19 mmol) in 1,4-dioxane (5 mL) and water (1 mL) was added to a 50 ml sealed tube with stirring. Sodium carbonate (290.41 mg, 2.74 mmol) was then added, and the reaction purged with nitrogen gas for 15 min. Pd(dppf)Cl₂ (111.79 mg, 137.00 μmol) was added, and the reaction was heated at 80° C. for 2 h. The reaction was filtered through Celite and washed with ethyl acetate (50 mL). The filtrate was concentrated and purified by flash column chromatography (silica gel 230-400 mesh, 25% ethyl acetate in pet-ether) to afford tert-butyl 4-[3-(2,6-dibenzyloxy-3-pyridyl)-1-methyl-indazol-6-yl]-3,3-difluoro-2,6-dihydropyridine-1-carboxylate (10, 487 mg, 722.00 μmol, 79% yield) as a yellow sticky solid. LCMS (ES+): m/z 639.2 [M+H]⁺.

### Step 6: ter-butyl 4-[3-(2,6-dioxo-3-piperidyl)-1-methyl-indazol-6-yl]-3,3-difluoro-piperidine-1-carboxylate (B-408)

tert-butyl 4-[3-(2,6-dioxo-3-piperidyl)-1-methyl-indazol-6-yl]-3,3-difluoro-piperidine-1-carboxylate (B-408, 300 mg, 636.15 μmol, 90% yield) was synthesized from tert-butyl 4-[3-(2,6-dibenzyloxy-3-pyridyl)-1-methyl-indazol-6-yl]-3,3-difluoro-2,6-dihydropyridine-1-carboxylate (10, 480 mg, 706.43 μmol) in a similar fashion to Compound B-103, except using 20 wt. % palladium hydroxide on carbon (545.66 mg, 777.08 μmol) and triturating the product with diethyl ether. LCMS (ES⁺): m/z 463.2 [M+H]⁺.

### 3-(6-(3,3-difluoropiperidin-4-yl)-1-methyl-1H-indazol-3-yl)piperidine-2,6-dione (B-409)

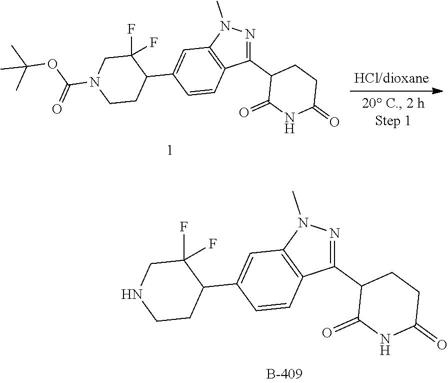 [SMILES: Cn1nc(C2CCC(=O)NC2=O)c2ccc(C3CCN(C(=O)OC(C)(C)C)CC3(F)F)cc21 ; Cn1nc(C2CCC(=O)NC2=O)c2ccc(C3CCNCC3(F)F)cc21]

### Step 1: 3-(6-(3,3-difluoropiperidin-4-yl)-1-methyl-1H-indazol-3-yl)piperidine-2,6-dione (B-409)

3-(6-(3,3-difluoropiperidin-4-yl)-1-methyl-1H-indazol-3-yl)piperidine-2,6-dione (B-409, 430 mg, 1.00 mmol, 93% yield, HCl salt) was synthesized from tert-butyl 4-(3-(2,6-dioxopiperidin-3-yl)-1-methyl-1H-indazol-6-yl)-3,3-difluoropiperidine-1-carboxy late (1, 0.5 g, 1.08 mmol) in a similar fashion to (6, step 3 of Compound A-148/Compound A-149), using HCl/dioxane (4 M, 2 mL) and stirring at 20° C. for 2 h.

LCMS (ESI): m/z 363.1 [M+H]⁺.

### 2-[1-[1-(2,6-dioxo-3-piperidyl)-3-methyl-2-oxo-benzimidazol-5-yl]-4-hydroxy-4-piperidyl]acetic acid (B-410)

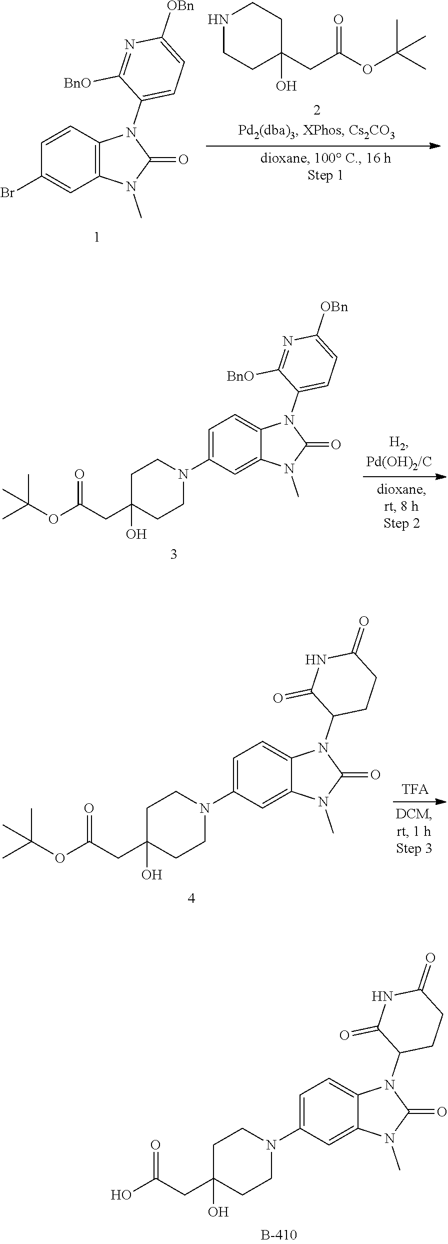

### Step 1: tert-butyl 2-[1-[1-(2,6-dibenzyloxy-3-pyridyl)-3-methyl-2-oxo-benzimidazol-5-yl]-4-hydroxy-4-piperidyl]acetate (3)

tert-butyl 2-[1-[1-(2,6-dibenzyloxy-3-pyridyl)-3-methyl-2-oxo-benzimidazol-5-yl]-4-hydroxy-4-piperidyl]acetate (3, 300 mg, 453.39 μmol, 49% yield) was synthesized from 5-bromo-1-(2,6-dibenzyloxy-3-pyridyl)-3-methyl-benzimidazol-2-one (1, 0.5 g, 930.22 μmol) and tert-butyl 2-(4-hydroxy-4-piperidyl)acetate (2, 240.32 mg, 1.12 mmol) in a similar fashion to Compound B-180, except using 0.1 eq. XPhos, 0.5 eq. tris(dibenzylideneacetone) dipalladium (0), and heating at 100° C.

LCMS (ES+): m/z 651.3 [M+H]⁺

### Step 2: tert-butyl 2-[1-[1-(2,6-dioxo-3-piperidyl)-3-methyl-2-oxo-benzimidazol-5-yl]-4-hydroxy-4-piperidyl]acetate (4)

tert-butyl 2-[1-[1-(2,6-dioxo-3-piperidyl)-3-methyl-2-oxo-benzimidazol-5-yl]-4-hydroxy-4-piperidyl]acetate (4, 0.170 mg, 358.76 μmol, 93% yield) was synthesized from tert-butyl 2-[1-[1-(2,6-dibenzyloxy-3-pyridyl)-3-methyl-2-oxo-benzimidazol-5-yl]-4-hydroxy-4-piperidyl]acetate (3, 250 mg, 384.17 μmol) in a similar fashion to Compound B-103, using palladium hydroxide on carbon (0.25 g, 384.17 μmol). The material was purified by silica gel flash column chromatography (230-400 mesh silica gel, 80-90% EtOAc in pet-ether). LCMS (ES+): m/z 473.3 [M+H]⁺.

### Step 3: 2-[1-[1-(2,6-dioxo-3-piperidyl)-3-methyl-2-oxo-benzimidazol-5-yl]-4-hydroxy-4-piperidyl]acetic acid (B-410)

2-[1-[1-(2,6-dioxo-3-piperidyl)-3-methyl-2-oxo-benzimidazol-5-yl]-4-hydroxy-4-piperidyl]acetic acid (B-410, 160 mg, 284.74 μmol, 79% yield, TFA salt) was synthesized from tert-butyl 2-[1-[1-(2,6-dioxo-3-piperidyl)-3-methyl-2-oxo-benzimidazol-5-yl]-4-hydroxy-4-piperidyl]acetate (4, 170 mg, 359.76 μmol) in a similar fashion to Compound A-26, except using 15 eq. TFA and triturating the product with ether. LCMS (ES+): m/z 417.0 [M+H]⁺.

### 3-[5-(azetidin-3-yl)-3-methyl-2-oxo-benzimidazol-1-yl]piperidine-2,6-dione (B-411)

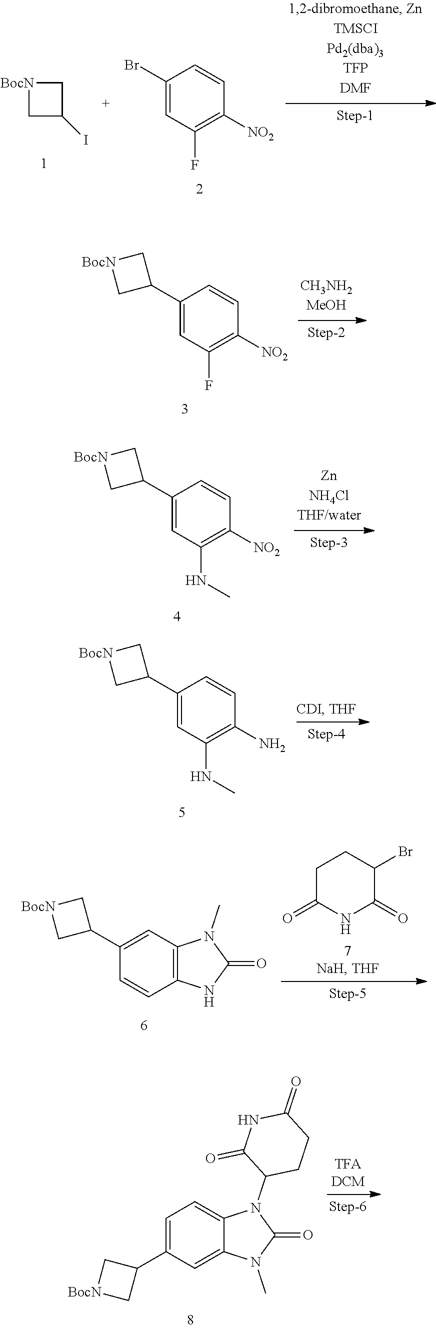

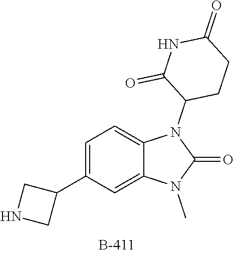 [SMILES: Cn1c(=O)n(C2CCC(=O)NC2=O)c2ccc(C3CNC3)cc21]

### Step 1: tert-butyl 3-(3-fluoro-4-nitrophenyl)azetidine-1-carboxylate (3)

1,2-dibromoethane (597.21 mg, 3.18 mmol, 273.95 μL) was added to a vigorously stirred suspension of zinc powder (325 mesh) (1.80 g, 27.55 mmol) in DMF (15 mL) under nitrogen atmosphere. The resulting suspension was heated at 70° C. for 10 min. Trimethyl silyl chloride (345.37 mg, 3.18 mmol, 403.47 μL) in DMF (15 mL) was added at rt and stirred for 45 min before a solution of tert-butyl 3-iodoazetidine-1-carboxylate (1, 6 g, 21.19 mmol) in DMF (15 mL) was added to the reaction mixture. The reaction was stirred at 40° C. for 45 min. Subsequently, a solution of (1E,4E)-1,5-diphenylpenta-1,4-dien-3-one; palladium (194.07 mg, 211.93 μmol), tri-2-furanylphosphine (TFP) (295.23 mg, 1.27 mmol) and 4-bromo-2-fluoro-1-nitrobenzene (2, 4.66 g, 21.19 mmol) in DMF (15 mL) was added and the reaction was heated at 70° C. for 3 h, after which it was quenched with brine solution (50 mL). The aqueous phase was extracted with ethyl acetate (2×200 mL) and the combined organic layers were dried over anhydrous sodium sulfate, filtered, and concentrated and purified by flash column chromatography (30% ethyl acetate in pet ether) to obtain tert-butyl 3-(3-fluoro-4-nitrophenyl)azetidine-1-carboxylate (3, 3.7 g, 12.07 mmol, 57% yield) as a yellow-orange solid. LCMS (ES+): m/z 197.0 [M−Boc+H]⁺.

### Step 2: tert-butyl 3-[3-(methylamino)-4-nitro-phenyl]azetidine-1-carboxylate (4)

In a 50 mL scaled tube, methyl amine solution (33% in ethanol) (6.11 g, 64.92 mmol, 6.80 mL) was added to a solution of tert-butyl 3-(3-fluoro-4-nitrophenyl)azetidine-1-carboxylate (3, 4.2 g, 12.98 mmol) in ethanol (25 mL) with stirring under nitrogen atmosphere at rt. The reaction was stirred at this temperature for 3 h. The reaction was diluted with ice cold water (50 mL) and the solid precipitation was filtered and washed with water and diethyl ether to yield product tert-butyl 3-[3-(methylamino)-4-nitro-phenyl]azetidine-1-carboxylate (4, 4 g, 12.83 mmol, 99% yield) as a yellow color solid. LCMS (ES⁺): m/z 252.0 [M−tBu+H]⁺.

### Step 3: tert-butyl 3-[4-amino-3-(methylamino)phenyl]azetidine-1-carboxylate (5)

To a stirred solution of tert-butyl 3-[3-(methylamino)-4-nitro-phenyl]azetidine-1-carboxylate (4, 3.5 g, 11.12 mmol) in 1:1 THF/water (30 mL) was added zinc powder (325 mesh) (3.64 g, 55.60 mmol). The suspension was cooled to 0° C. and ammonium chloride (2.97 g, 55.60 mmol) was added before the reaction was stirred for 1 h at rt. The reaction mixture was diluted with THE and filtered through a pad of Celite. The filtrate was concentrated under reduced pressure and diluted with DCM (150 mL). The organic layer was washed with sodium bicarbonate solution (50 mL), followed by water (50 mL) and brine solution (30 mL). It was then dried over anhydrous sodium sulfate and concentrated in vacuo to afford tert-butyl 3-[4-amino-3-(methylamino)phenyl]azetidine-1-carboxylate (5, 3.1 g, 10.79 mmol, 97% yield) as a brown gum. LCMS (ES+): m/z 222.0 [M−tBu+H]⁺.

### Step 4: tert-butyl 3-(3-methyl-2-oxo-2,3-dihydro-1H-benzo[d]imidazol-5-yl)azetidine-1-carboxylate (6)

In a 250 mL single-neck round-bottom flask, a well-stirred solution of tert-butyl 3-(4-amino-3-(methylamino)phenyl)azetidine-1-carboxylate (5, 3.2 g, 9.46 mmol) in THF (30 mL) was added carbonyldiimidazole (2.30 g, 14.19 mmol) under inert atmosphere. The reaction mixture was stirred for 3 h. The reaction was concentrated and purified by column chromatography (silica gel 230-400 mesh, 0-100% ethyl acetate/pet ether) to afford tert-butyl 3-(3-methyl-2-oxo-2,3-dihydro-1H-benzo[d]imidazol-5-yl)azetidine-1-carboxylate (6, 2.7 g, 8.68 mmol, 92% yield) as a brown color solid. LCMS (ES⁺): m/z 304.0 [M+H]⁺.

### Step 5: tert-butyl 3-[1-(2,6-dioxo-3-piperidyl)-3-methyl-2-oxo-benzimidazol-S-yl]azetidine-1-carboxylate (8)

To the stirred solution of tert-butyl 3-(3-methyl-2-oxo-1H-benzimidazol-5-yl)azetidine-1-carboxylate (6, 4 g, 12.85 mmol) in THF (100 mL) was added sodium hydride (60% dispersion in mineral oil) (2.36 g, 61.56 mmol) at 0° C. and the reaction was stirred at rt for 30 min. Then 3-bromopiperidine-2,6-dione (7, 7.40 g, 38.56 mmol) in THF (100 mL) was added at 0° C. and the reaction was heated at 60° C. for 19 h. The reaction was quenched with saturated ammonium chloride solution (200 mL) and extracted with ethyl acetate (3×100 mL). The organic layers were combined, dried over sodium sulfate, concentrated and purified by column chromatography (silica gel, 0-100% ethyl acetate in pet-ether) to afford tert-butyl 3-[1-(2,6-dioxo-3-piperidyl)-3-methyl-2-oxo-benzimidazol-5-yl]azetidine-1-carboxylate (8, 2.88 g, 6.41 mmol, 50% yield) as a yellow solid. LCMS (ES⁻): m/z 413.2 [M−H]⁻.

### Step 6: 3-[5-(azetidin-3-yl)-3-methyl-2-oxo-benzimidazol-1-yl]piperidine-2,6-dione (B-411)

3-[5-(azetidin-3-yl)-3-methyl-2-oxo-benzimidazol-1-yl]piperidine-2,6-dione (B-411, 2.23 g, 3.90 mmol, 61% yield, TFA salt) was synthesized from tert-butyl 3-[1-(2,6-dioxo-3-piperidyl)-3-methyl-2-oxo-benzimidazol-5-yl]azetidine-1-carboxylate (8, 2.88 g, 6.41 mmol) in a similar fashion to Compound A-62, except using 1 eq. TFA. The material was purified by reverse-phase column chromatography (C18, 120 g, 0.1% formic acid in water/acetonitrile). LCMS (ES⁺): m/z 315.1 [M+H]⁺.

### 2-[3-[1-(2,6-dioxo-3-piperidyl)-3-methyl-2-oxo-benzimidazol-5-yl]azetidin-1-yl]acetic acid (B-412)

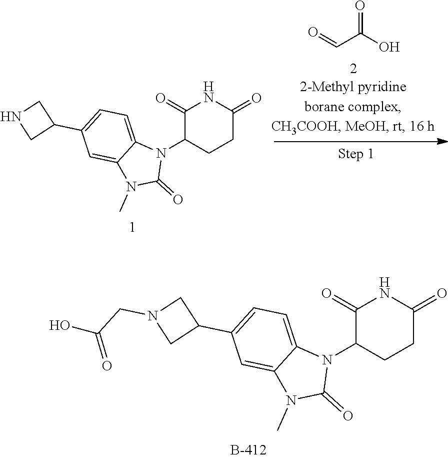 [SMILES: O=CC(=O)O ; Cn1c(=O)n(C2CCC(=O)NC2=O)c2ccc(C3CNC3)cc21 ; Cn1c(=O)n(C2CCC(=O)NC2=O)c2ccc(C3CN(CC(=O)O)C3)cc21]

### Step 1: 2-[3-[1-(2,6-dioxo-3-piperidyl)-3-methyl-2-oxo-benzimidazol-5-yl]azetidin-1-yl]acetic acid (B-412)

Into a 50 mL single neck round bottom flask containing a well-stirred solution of 3-[5-(azetidin-3-yl)-3-methyl-2-oxo-benzimidazol-1-yl]piperidine-2,6-dione (1, 600 mg, 1.43 mmol), glyoxalic acid monohydrate (2, 263.56 mg, 2.86 mmol) in anhydrous methanol (10 mL) was added acetic acid (859.69 mg, 14.32 mmol, 818.75 μL) and the mixture was stirred at rt for 15 min. Then 2-methylpyridine borane complex (229.68 mg, 2.15 mmol) was added and continued stirring at rt. After 16 h, the mixture was concentrated and purified by reverse phase prep HPLC [Purification method: Column: X-Select C18 (150×19) mm, 5 micron; Mobile phase: 0.1% Formic acid in water; Mobile phase B: MeCN] to afford 2-[3-[1-(2,6-dioxo-3-piperidyl)-3-methyl-2-oxo-benzimidazol-5-yl]azetidin-1-yl]acetic acid (B-412, 170 mg, 377.87 μmol, 26% yield, Formic acid salt) as an off-white solid.

LCMS (ES+): m/z 373.1 [M+H]⁺.

### tert-butyl 4-[3-tert-butyl-1-(2,6-dioxo-3-piperidyl)-2-oxo-benzimidazol-5-yl]piperidine-1-carboxylate (B-413)

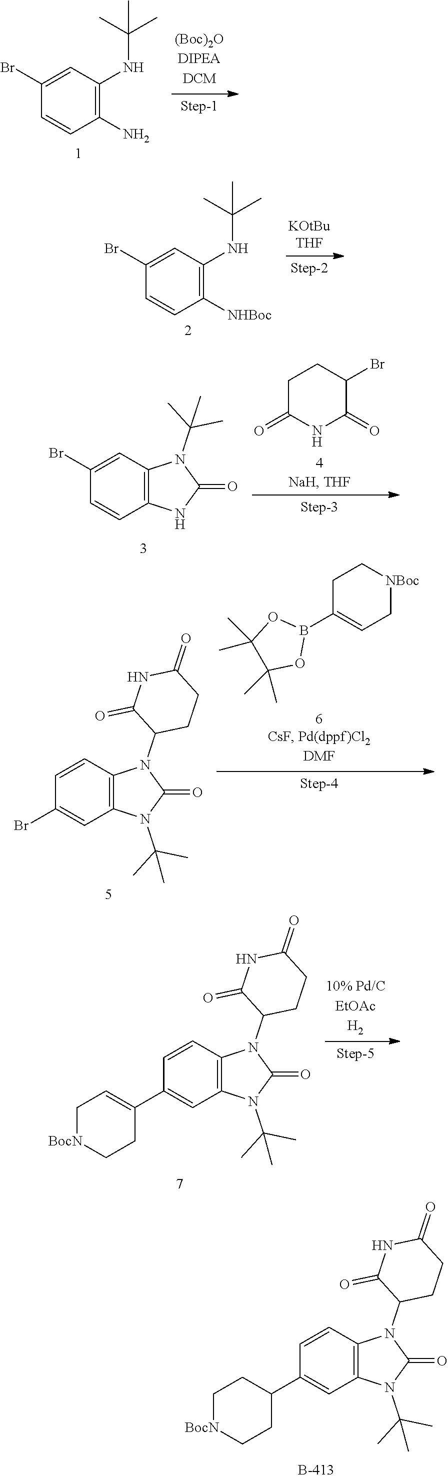

### Step 1: tert-butyl N-[4-bromo-2-(tert-butylamino)phenyl]carbamate (2)

To a stirred solution of 4-bromo-N2-tert-butyl-benzene-1,2-diamine (1, 17 g, 69.92 mmol) in DCM (300 mL) was added N-ethyl-N-isopropyl-propan-2-amine (27.11 g, 209.75 mmol, 36.54 mL) followed by tert-butoxycarbonyl tert-butyl carbonate (22.89 g, 104.88 mmol, 24.07 mL) dropwise at 0° C. The resulting solution was stirred at rt for 16 h. The mixture was concentrated and purified by column chromatography (silica gel, 15-20% ethyl acetate in hexane) to afford tert-butyl N-[4-bromo-2-(tert-butylamino)phenyl]carbamate (2, 20 g, 58.27 mmol, 83% yield) as an off-white solid. The material was used in the next step without further characterization.

### Step 2: 5-bromo-3-tert-butyl-1H-benzimidazol-2-one (3)

To a stirred solution of tert-butyl N-[4-bromo-2-(tert-butylamino)phenyl]carbamate (2, 20 g, 58.27 mmol) in THF (150 mL) was added potassium; 2-methylpropan-2-olate (7.19 g, 64.09 mmol) portionwise at 0° C. and the reaction was stirred at 25° C. for 2 h. The solvent was evaporated, and the residue was extracted with ethyl acetate. The combined organic layers were washed with water, brine, dried over anhydrous sodium sulfate, filtered, concentrated and purified by column chromatography (silica gel, 40% ethyl acetate in hexane) to afford 5-bromo-3-tert-butyl-1H-benzimidazol-2-one (3, 13.4 g, 49.29 mmol, 85% yield) as an off-white solid. LCMS (ES+): m/z 269.1 [M+H]⁺.

### Step 3: 3-(5-bromo-3-tert-butyl-2-oxo-benzimidazol-1-yl)piperidine-2,6-dione (5)

To a stirred solution of 5-bromo-3-tert-butyl-1H-benzimidazol-2-one (4, 5 g, 18.58 mmol) in THF (150 mL) was slowly added sodium hydride (60% dispersion in mineral oil) (7.12 g, 185.78 mmol) portionwise at 0° C. The reaction was stirred for 30 min at rt. 3-Bromopiperidine-2,6-dione (4, 17.84 g, 92.89 mmol) was then added portionwise at rt and the reaction was heated to 60° C. for 5 h. The reaction was quenched with crushed ice and extracted with ethyl acetate. The combined organic layers were washed with water, brine, dried over anhydrous sodium sulfate, filtered, concentrated, and purified by trituration with 15% ethyl acetate in hexane to afford 3-(5-bromo-3-tert-butyl-2-oxo-benzimidazol-1-yl)piperidine-2,6-dione (5, 4.4 g, 11.25 mmol, 61% yield) as a white solid. ¹H NMR (400 MHz, DMSO-d₆) d 11.07 (bs, 1H), 7.61 (s, 1H), 7.20 (d, J=8.0 Hz, 1H), 7.07 (d, J=8.4 Hz, 1H), 5.34-5.31 (m, 1H), 2.89-2.81 (m, 1H), 2.67-2.58 (m, 2H), 2.00-1.97 (m, 1H), 1.70 (s, 9H).

### Step 4: tert-butyl 4-[3-tert-butyl-1-(2,6-dioxo-3-piperidyl)-2-oxo-benzimidazol-5-yl]-3,6-dihydro-2H-pyridine-1-carboxylate (7)

tert-butyl 4-[3-tert-butyl-1-(2,6-dioxo-3-piperidyl)-2-oxo-benzimidazol-5-yl]-3,6-dihydro-2H-pyridine-1-carboxylate (7, 2.1 g, 4.10 mmol, 45% yield) was synthesized from 3-(5-bromo-3-tert-butyl-2-oxo-benzimidazol-1-yl)piperidine-2,6-dione (5, 3.5 g, 9.20 mmol) and tert-butyl 4-(4,4,5,5-tetramethyl-1,3,2-dioxaborolan-2-yl)-3,6-dihydro-2H-pyridine-1-carboxylate (6, 5.69 g, 18.41 mmol) in a similar fashion to Compound B-133, except eq. and using 2.5 CsF 0.1 eq. [1,1′-Bis(diphenylphosphino)ferrocene]dichloropalladium(II) complex with dichloromethane. LCMS (ES⁺): m/z 483.4 [M+H]⁺.

### Step 5: tert-butyl 4-[3-tert-butyl-1-(2,6-dioxo-3-piperidyl)-2-oxo-benzimidazol-5-yl]piperidine-1-carboxylate (B-413)

tert-butyl 4-[3-tert-butyl-1-(2,6-dioxo-3-piperidyl)-2-oxo-benzimidazol-5-yl]piperidine-1-carboxylate (B-413, 1.2 g, 2.44 mmol, 65% yield) was synthesized from tert-butyl 4-[3-tert-butyl-1-(2,6-dioxo-3-piperidyl)-2-oxo-benzimidazol-5-yl]-3,6-dihydro-2H-pyridine-1-carboxylate (7, 1.8 g, 3.73 mmol) in a similar fashion to Compound B-6. The material was purified by CombiFlash® (60% ethyl acetate in hexane). ¹H NMR (400 MHz, DMSO-d₆) d 11.05 (s, 1H), 7.32 (s, 1H), 6.97 (d, J=7.6 Hz, 1H), 6.89 (d, J=7.9 Hz, 1H), 5.30-5.28 (m, 1H), 4.08-4.04 (m, 2H), 2.90-2.57 (m, 6H), 1.99-1.93 (m, 1H), 1.76-1.71 (m, 11H), 1.55-1.46 (m, 2H), 1.42 (s, 9H).

### 3-[3-tert-butyl-2-oxo-5-(4-piperidyl)benzimidazol-1-yl]piperidine-2,6-dione (B-414)

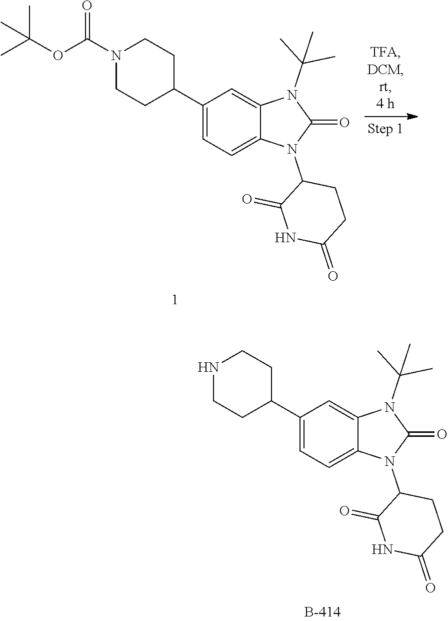 [SMILES: CC(C)(C)OC(=O)N1CCC(c2ccc3c(c2)n(C(C)(C)C)c(=O)n3C2CCC(=O)NC2=O)CC1 ; CC(C)(C)n1c(=O)n(C2CCC(=O)NC2=O)c2ccc(C3CCNCC3)cc21]

### Step 1: 3-[3-tert-butyl-2-oxo-5-(4-piperidyl)benzimidazol-1-yl]piperidine-2,6-dione (B-414)

3-[3-tert-butyl-2-oxo-5-(4-piperidyl)benzimidazol-1-yl]piperidine-2,6-dione (B-414, 130 mg, 255.20 μmol, 95% yield, TFA salt) was synthesized from tert-butyl 4-[3-tert-butyl-1-(2,6-dioxo-3-piperidyl)-2-oxo-benzimidazol-5-yl]piperidine-1-carboxylate (1, 130 mg, 268.27 μmol) in a similar fashion to Compound A-62, except using 19 eq. TFA and triturating the product with diethyl ether. LCMS (ES+): m/z 385.22 [M+H]⁺.

### 3-(2-oxo-6-(4-oxopiperidin-1-yl)benzo[cd]indol-1(2H)-yl)piperidine-2,6-dione (B-415)

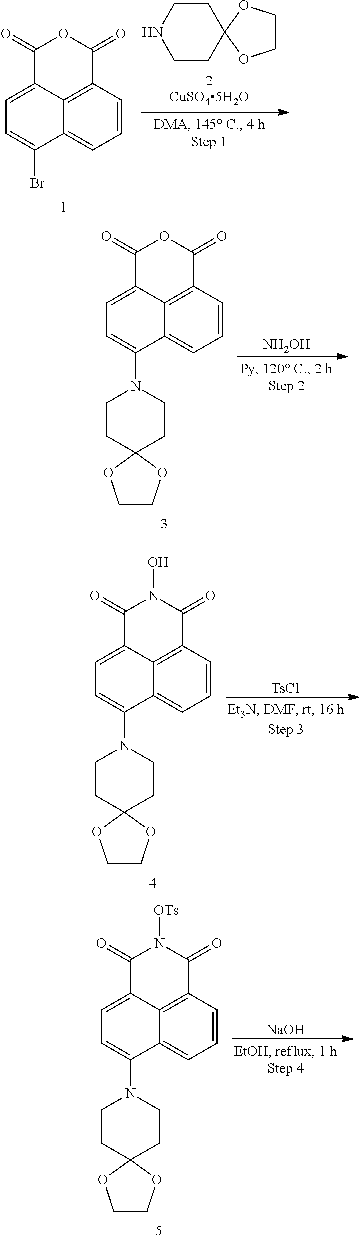

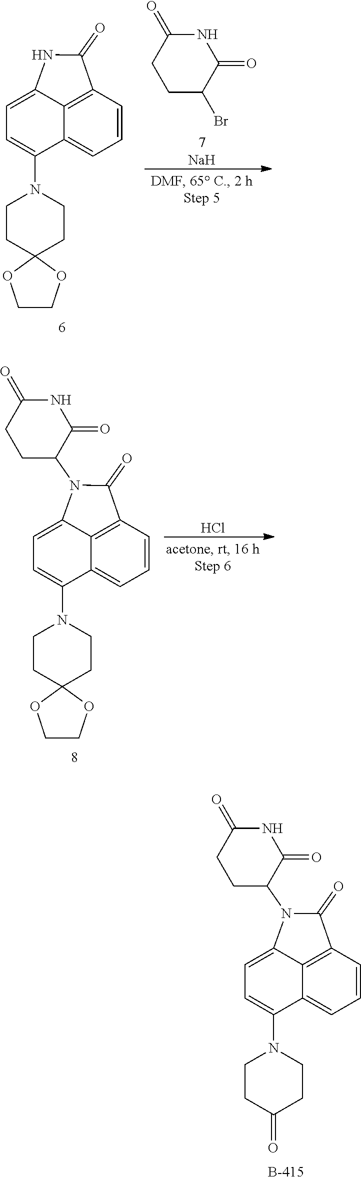

### Step 1: 6-(1,4-dioxa-8-azaspiro[4.5]decan-8-yl)-1H,3H-benzo[de]isochromene-1,3-dione (3)

Into a 250 mL sealed-tube containing a well-stirred solution of 6-bromo-1H,3H-benzo[de]isochromene-1,3-dione (1, 5 g, 18.05 mmol) in DMA (50 mL) were added copper(II) sulfate pentahydrate (2.25 g, 9.02 mmol) and 1,4-dioxa-8-azaspiro[4.5]decane (6.46 g, 45.11 mmol, 5.77 mL). The reaction mixture was stirred for 4 h at 145° C. The reaction mixture was diluted with water (100 mL) and the precipitated solid was filtered, washed with water (40 mL) and dried over vacuum to afford 6-(1,4-dioxa-8-azaspiro[4.5]decan-8-yl)-1H,3H-benzo[de]isochromene-1,3-dione (3, 6 g, 17.49 mmol, 97% yield) as yellow solid. LCMS (ES+): m/z 340.2 [M+H]⁺.

### Step 2: 2-hydroxy-6-(1,4-dioxa-8-azaspiro[4.5]decan-8-yl)-1H-benzo[de]isoquinoline-1,3(2H)-dione (4)

Into a 50 mL single neck round bottom flask containing a well-stirred solution of 6-(1,4-dioxa-8-azaspiro[4.5]decan-8-yl)-1H,3H-benzo[de]isochromene-1,3-dione (3, 1 g, 2.91 mmol) in pyridine (6 mL) was added hydroxylamine hydrochloride (303.79 mg, 4.37 mmol). The reaction mixture was stirred at 120° C. for 2 h. The solvent was evaporated under reduced pressure to afford 2-hydroxy-6-(1,4-dioxa-8-azaspiro[4.5]decan-8-yl)-1H-benzo[de]isoquinoline-1,3(2H)-dione (4, 1.2 g, 2.24 mmol, 77% yield, 66% purity) as yellow sticky solid. LCMS (ES+): m/z 355.2 [M+H]⁺.

### Step 3: 1,3-dioxo-6-(1,4-dioxa-8-azaspiro[4.5]decan-8-yl)-1H-benzo[de]isoquinolin-2(3H)-yl 4-methylbenzenesulfonate (5)

Into a 250 mL single neck round bottom flask containing a well-stirred solution of 2-hydroxy-6-(1,4-dioxa-8-azaspiro[4.5]decan-8-yl)-1H-benzo[de]isoquinoline-1,3(2H)-dione (4, 5 g, 6.60 mmol) in DMF (50 mL) were added triethyl amine (3.34 g, 33.02 mmol, 4.60 mL) and TsCl (1.26 g, 6.60 mmol) at 0° C. The reaction mixture was stirred at ambient temperature for 16 h. Afterwards, water (50 mL) was added to the reaction mixture and the precipitated solid was filtered, washed with water and dried under vacuum to afford 1,3-dioxo-6-(1,4-dioxa-8-azaspiro[4.5]decan-8-yl)-1H-benzo[de]isoquinolin-2(3H)-yl 4-methylbenzenesulfonate (5, 4 g, 5.87 mmol, 89% yield, 75% purity) as orange solid. The material was used in the next step without further purification. LCMS (ES+): m/z 509.0 [M+H]⁺.

### Step 4:6-(1,4-dioxa-8-azaspiro[4.5]decan-8-yl)benzo[cd]indol-2 (1H)-one (6)

Into a 250 mL single neck round bottom flask containing a suspension of 1,3-dioxo-6-(1,4-dioxa-8-azaspiro[4.5]decan-8-yl)-1H-benzo[de]isoquinolin-2(3H)-yl 4-methylbenzenesulfonate (5, 4 g, 5.87 mmol) in ethanol (20 mL) was added 10% NaOH solution (234.69 mg, 5.87 mmol, 20 mL) and the resulting mixture was heated at reflux for 1 h. Afterwards, ethanol was evaporated and water (50 mL) was added to the reaction mixture. The precipitated solid filtered was washed with water (20 mL), dried under reduced pressure and purified by silica gel (230-400 mesh) flash column chromatography (70% EtOAc in pet ether) to afford 6-(1,4-dioxa-8-azaspiro[4.5]decan-8-yl)benzo[cd]indol-2 (1H)-one (6, 1.0 g, 2.32 mmol, 40% yield, 72% purity) as an orange solid. LCMS (ES+): m/z 311.2 [M+H]⁺.

### Step 5: 3-(2-oxo-6-(1,4-dioxa-8-azaspiro[4.5]decan-8-yl)benzo[cd]indol-1(2H)-yl)piperidine-2,6-dione (8)

Into a 250 mL two neck round bottom flask containing a well-stirred suspension of sodium hydride (60% dispersion in mineral oil) (1.02 g, 25.49 mmol) in anhydrous THF (15 mL) at 0° C. was added 6-(1,4-dioxa-8-azaspiro[4.5]decan-8-yl)benzo[cd]indol-2 (1H)-one (6, 920 mg, 2.55 mmol) in dry THF (15 mL) and the resulting mixture was stirred for 1 h at rt. Subsequently, 3-bromopiperidine-2,6-dione (7, 1.96 g, 10.20 mmol) in dry THF (15 mL) was added and the mixture was heated at 65° C. for 2 h. The reaction mixture was quenched with saturated ammonium chloride solution (10 mL) and extracted with EtOAc (3×100 mL). The combined organic layers were dried over sodium sulfate, filtered, concentrated and purified by silica gel (230-400 mesh) flash column chromatography (70-100% EtOAc in Pet ether) to afford 3-(2-oxo-6-(1,4-dioxa-8-azaspiro[4.5]decan-8-yl)benzo[cd]indol-1(2H)-yl)piperidine-2,6-dione (8, 800 mg, 1.38 mmol, 54% yield, 73% purity) as a red solid. LCMS (ES+): 422.0 [M+H]⁺.

### Step 6: 3-(2-oxo-6-(4-oxopiperidin-1-yl)benzo[cd]indol-1(2H)-yl)piperidine-2,6-dione (B-415)

Into a 250 mL single neck round bottom flask containing a well-stirred solution of 3-(2-oxo-6-(1,4-dioxa-8-azaspiro[4.5]decan-8-yl)benzo[cd]indol-1(2H)-yl)piperidine-2,6-dione 8, 700 mg, 946.74 μmol) in acetone (20 mL) was added 6N HCl solution (13.68 g, 47.34 mmol, 8 mL) dropwise at 0° C. The resulting mixture was stirred at ambient temperature for 16 h. The reaction mixture was quenched with 10% NaHCO₃ solution at 0° C. to afford the solid that was filtered and dried over vacuum. Purification by reverse-phase column chromatography [Column: REDISEP-C18-120 g; Mobile Phase A: 0.1% FA in Water and Mobile Phase B: MeCN] to afford 3-(2-oxo-6-(4-oxopiperidin-1-yl)benzo[cd]indol-1(2H)-yl)piperidine-2,6-dione (B-415, 100 mg, 218.22 μmol, 23% yield, formic acid salt) as a red solid. LCMS (ES+): 378.2 [M+H]⁺.

### 2-(3-((1-(2,6-dioxopiperidin-3-yl)-3-methyl-2-oxo-2,3-dihydro-1H-benzo[d]imidazol-5-yl)amino)phenyl)acetic acid (B-416)

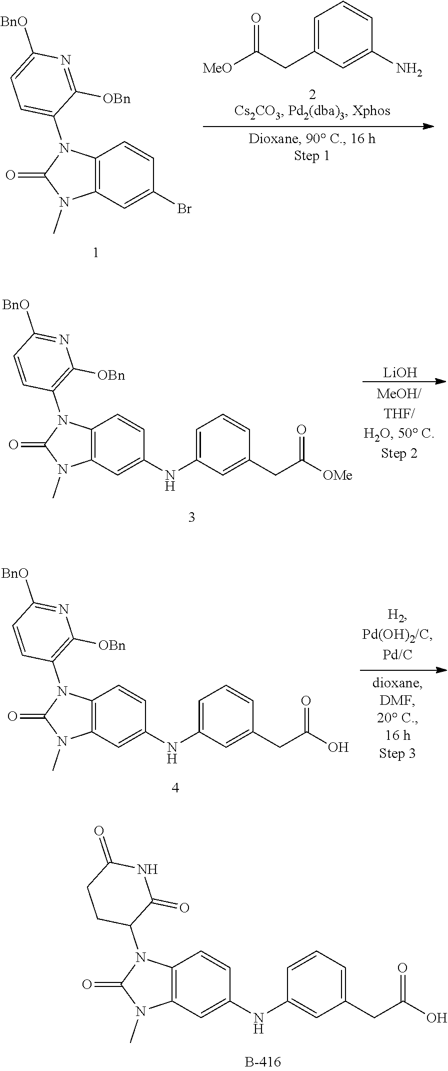 [SMILES: Cn1c(=O)n(-c2ccc(OCc3ccccc3)nc2OCc2ccccc2)c2ccc(Br)cc21 ; COC(=O)Cc1cccc(N)c1 ; COC(=O)Cc1cccc(Nc2ccc3c(c2)n(C)c(=O)n3-c2ccc(OCc3ccccc3)nc2OCc2ccccc2)c1 ; "LiOH" ; Cn1c(=O)n(-c2ccc(OCc3ccccc3)nc2OCc2ccccc2)c2ccc(Nc3cccc(CC(=O)O)c3)cc21 ; Cn1c(=O)n(C2CCC(=O)NC2=O)c2ccc(Nc3cccc(CC(=O)O)c3)cc21]

### Step 1: methyl 2-(3-((1-(2,6-bis(benzyloxy)pyridin-3-yl)-3-methyl-2-oxo-2,3-dihydro-1H-benzo[d]imidazol-5-yl)amino)phenyl)acetate (3)

methyl 2-(3-((1-(2,6-bis(benzyloxy)pyridin-3-yl)-3-methyl-2-oxo-2,3-dihydro-1H-benzo[d]imidazol-5-yl)amino)phenyl)acetate (3, 0.7 g, 0.99 mmol, 85% yield) was synthesized from 1-(2,6-bis(benzyloxy)pyridin-3-yl)-5-bromo-3-methyl-1H-benzo[d]imidazol-2(3H)-one (1, 784.6 mg, 1.51 mmol) and methyl 2-(3-aminophenyl)acetate (2, 250.0 mg, 3.87 mmol) in a similar fashion to Compound B-180, except using 2 eq. cesium carbonate, 0.1 eq. tris(dibenzylideneacetone) dipalladium (0) and 0.2 eq. XPhos.

LCMS (ESI): m/z 601.1 [M+H]⁺.

HNMR (400 MHz, DMSO-d6) δ=8.03 (s, 1H), 7.83-7.77 (m, 1H), 7.48-7.42 (m, 2H), 7.41-7.34 (m, 3H), 7.32-7.23 (m, 5H), 7.12 (s, 1H), 7.00-6.86 (m, 3H), 6.73 (dd, J=2.0, 8.4 Hz, 1H), 6.62 (s, 1H), 6.60-6.58 (m, 1H), 5.46-5.31 (m, 4H), 3.60 (s, 3H), 3.57 (s, 2H), 3.36-3.33 (m, 3H), 3.30-3.29 (m, 1H).

### Step 2: 2-(3-((1-(2,6-bis(benzyloxy)pyridin-3-yl)-3-methyl-2-oxo-2,3-dihydro-1H-benzo[d]imidazol-5-yl)amino)phenyl)acetic acid (4)

2-(3-((1-(2,6-bis(benzyloxy)pyridin-3-yl)-3-methyl-2-oxo-2,3-dihydro-1H-benzo[d]imidazol-5-yl)amino)phenyl)acetic acid (4, 0.3 g, 0.4 mmol, 78% yield) was synthesized from methyl 2-(3-((1-(2,6-bis(benzyloxy)pyridin-3-yl)-3-methyl-2-oxo-2,3-dihydro-1H-benzo[d]imidazol-5-yl)amino)phenyl)acetate (3, 0.6 g, 1.0 mmol) in a similar fashion to Compound A-9 except using 15 eq. LiOH·H₂O and heating to 50° C. The mixture was adjusted pH to 5 with 2 N HCl aqueous. The mixture was extracted with ethyl acetate (10 mL). The organic phase was washed with brine (10 mL), dried with anhydrous Na₂SO₄, filtered and concentrated in vacuum. The residue was purified by column chromatography (SiO₂, Petroleum ether/Ethyl acetate=100/1 to 1/1). LCMS (ESI): m/z 586.8 [M+H]+. HNMR (400 MHz, DMSO-d6) δ=12.25 (br s, 1H), 8.02 (s, 1H), 7.80 (d, J=8.3 Hz, 1H), 7.47-7.42 (m, 2H), 7.41-7.33 (m, 3H), 7.31-7.25 (m, 5H), 7.12 (t, J=7.8 Hz, 1H), 6.99-6.92 (m, 2H), 6.87 (br d, J=8.1 Hz, 1H), 6.73 (dd, J=1.9, 8.4 Hz, 1H), 6.65-6.61 (m, 1H), 6.60-6.56 (m, 1H), 5.45-5.32 (m, 4H), 3.46 (s, 2H), 3.35-3.33 (m, 3H), 3.29 (br s, 1H).

### Step 3: 2-(3-((1-(2,6-dioxopiperidin-3-yl)-3-methyl-2-oxo-2,3-dihydro-1H-benzo[d]imidazol-5-ylamino)phenyl)acetic acid (B-416)

2-(3-((1-(2,6-dioxopiperidin-3-yl)-3-methyl-2-oxo-2,3-dihydro-1H-benzo[d]imidazol-5-yl)amino)phenyl)acetic acid (B-416, 65 mg, 0.16 mmol, 72% yield) was synthesized from 2-(3-((1-(2,6-bis(benzyloxy)pyridin-3-yl)-3-methyl-2-oxo-2,3-dihydro-1H-benzo[d]imidazol-5-yl)amino)phenyl)acetic acid (4, 130 mg, 221.60 μmol) in a similar fashion to (4, step 2 of Compound B-391. The residue was purified by reverse phase HPLC (using TFA modifier).

1H NMR (400 MHz, DMSO-d6) δ=12.32 (dt, J=2.9, 5.4 Hz, 1H), 11.08 (s, 1H), 8.00 (s, 1H), 7.11 (t, J=7.8 Hz, 1H), 7.00 (d, J=8.4 Hz, 1H), 6.92 (d, J=2.0 Hz, 2H), 6.87 (br d, J=8.3 Hz, 1H), 6.76 (dd, J=1.9, 8.4 Hz, 1H), 6.63 (d, J=7.5 Hz, 1H), 5.32 (dd, J=5.3, 12.7 Hz, 1H), 3.46 (s, 2H), 3.29 (s, 3H), 2.96-2.84 (m, 1H), 2.76-2.62 (m, 2H), 2.05-1.99 (m, 1H).

LCMS (ESI): m/z 409.1 [M+H]+.

### 2-(3-(1-(2,6-dioxopiperidin-3-yl)-3-methyl-2-oxo-2,3-dihydro-1H-benzo[d]imidazol-5-yl)phenyl)acetic acid (B-417)

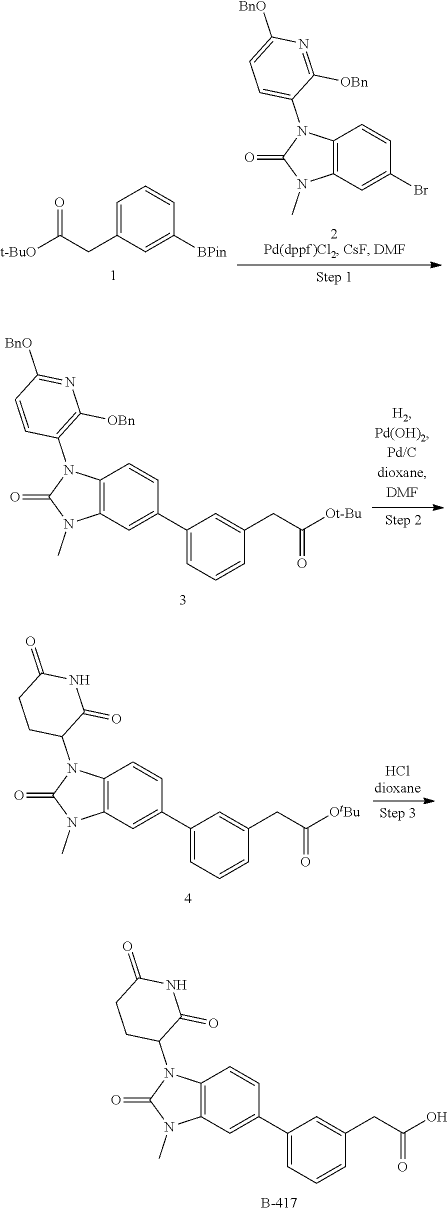

### Step 1: tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (3)

tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (3, 250 mg, 378.35 μmol, 39% yield) was synthesized from 1-(2,6-bis(benzyloxy)pyridin-3-yl)-5-bromo-3-methyl-1H-benzo[d]imidazol-2(3H)-one (2, 500 mg, 968.27 μmol) and tert-butyl 2-(3-(4,4,5,5-tetramethyl-1,3,2-dioxaborolan-2-yl)phenyl)acetate (1, 450 mg, 1.41 mmol) in a similar fashion to Compound B-133, except using 0.05 eq Pd(dppf)Cl₂.

LCMS (ESI): m/z 628.5 [M+H]⁺

### Step 2: tert-butyl 2-(3-(1-(2,6-dioxopiperidin-3-yl)-3-methyl-2-oxo-2,3-dihydro-1H-benzo[d]imidazol-5-yl)phenyl)acetate (4)

tert-butyl 2-(3-(1-(2,6-dioxopiperidin-3-yl)-3-methyl-2-oxo-2,3-dihydro-1H-benzo[d]imidazol-5-yl)phenyl)acetate (4, 130 mg, 259.83 μmol, 74% yield) was synthesized from tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (3, 220 mg, 350.47 μmol) in a similar fashion to (4, step 2 of Compound B-391, except using 10 wt. % Pd/C (40 mg) and 10 wt. % Pd(OH)₂/C (40 mg), and stirring at 30° C. for 16 h under H₂ (15 Psi). The material was purified by reversed phase column chromatography (formic acid condition). LCMS (ESI): m/z 450.4 [M+H]⁺.

### Step 3: 2-(3-(1-(2,6-dioxopiperidin-3-yl)-3-methyl-2-oxo-2,3-dihydro-1H-benzo[d]imidazol-5-yl)phenyl)acetic acid (B-417)

To a solution of tert-butyl 2-(3-(1-(2,6-dioxopiperidin-3-yl)-3-methyl-2-oxo-2,3-dihydro-1H-benzo[d]imidazol-5-yl)phenyl)acetate (4, 130 mg, 289.21 μmol) in dioxane (4 mL) was added HCl/dioxane (4 M, 2 mL). The mixture was stirred at 20° C. for 16 h. The reaction mixture was concentrated under reduced pressure. The residue was purified by reversed phase column (FA) to afford 2-(3-(1-(2,6-dioxopiperidin-3-yl)-3-methyl-2-oxo-2,3-dihydro-1H-benzo[d]imidazol-5-yl)phenyl)acetic acid (B-417, 40 mg, 101.68 μmol, 35% yield) as a white solid LCMS (ESI): m/z 394.4 [M+H]⁺.

### tert-butyl 4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (B-418)

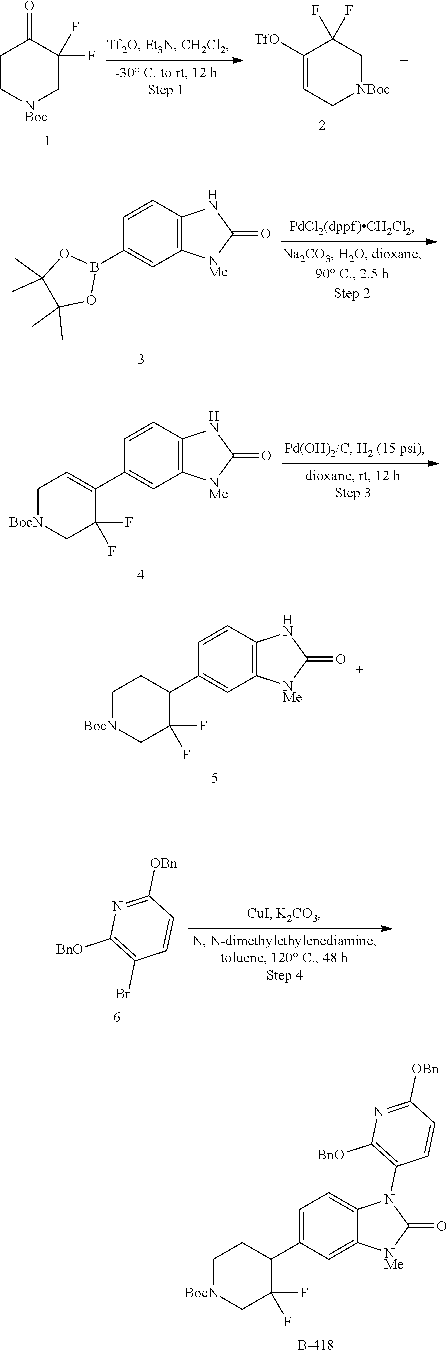

### Step 1: tert-butyl 3,3-difluoro-4-(((trifluoromethyl)sulfonyl)oxy)-3,6-dihydropyridine-1(2H)-carboxylate (2)

To a solution of tert-butyl 3,3-difluoro-4-oxopiperidine-1-carboxylate (1, 49 g, 208 mmol) in dichloromethane (490 mL) was added trimethylamine (87 mL, 625 mmol) followed by trifluoromethanesulfonic anhydride (52.8 mL, 312 mmol) at −30° C. The reaction mixture was warmed to rt and stirred for 12 h. Batches were combined for the workup. The mixture was quenched with aqueous saturated NaHCO₃ (1000 mL) and extracted with dichloromethane (3×500 mL). The combined organic phases were washed with brine (2×500 mL), dried over Na₂SO₄, filtered and concentrated under reduced pressure. The residue was purified by column chromatography on silica gel (petroleum ether:ethyl acetate=1:0) to give tert-butyl 3,3-difluoro-4-(((trifluoromethyl)sulfonyl)oxy)-3,6-dihydropyridine-1(2H)-carboxylate (2, 88 g, 51% yield) as yellow oil. ¹H NMR (400 MHz, DMSO-d₆) δ 6.83 (br s, 1H) 4.24 (br d, J=3.8 Hz, 2H) 3.96-4.12 (m, 2H) 1.42 (s, 9H); LCMS (ESI+, TFA): m/z 268.0 (M−CO₂t−Bu), rt=2.17 min.

### Step 2: tert-butyl 3,3-difluoro-4-(3-methyl-2-oxo-2,3-dihydro-1H-benzo[d]imidazol-5-yl)-3,6-dihydropyridine-1(2H)-carboxylate (4)

To a solution of tert-butyl 3,3-difluoro-4-(((trifluoromethyl)sulfonyl)oxy)-3,6-dihydropyridine-1(2H)-carboxylate (2, 30 g, 73.5 mmol) and 1-methyl-6-(4,4,5,5-tetramethyl-1,3,2-dioxaborolan-2-yl)-1,3-dihydro-2H-benzo[d]imidazol-2-one (3, 22.39 g, 73.5 mmol) in dioxane (300 mL) was added a solution of Na₂CO₃ (7.79 g, 73.5 mmol) in water (60.0 mL). The suspension was degassed and purged with N₂ for 3 times and then 1,1′-bis(diphenylphosphino)ferrocene-palladium(II)dichloride dichloromethane complex (6.00 g, 7.35 mmol) was added to the reaction. The suspension was degassed and purged with N₂ for 3 times then heated to 90° C. and stirred for 2.5 h. Similar reactions were combined and treated to the following workup. The mixture was cooled to rt and poured into water (1000 mL). The aqueous layer was extracted with ethyl acetate (3×500 mL) and the combined organic phases were washed with water (2×300 mL), dried over Na₂SO₄, filtered and concentrated under reduced pressure. The residue was purified by column chromatography on silica gel (petroleum ether:ethyl acetate=2:1) to give tert-butyl 3,3-difluoro-4-(3-methyl-2-oxo-2,3-dihydro-1H-benzo[d]imidazol-5-yl)-3,6-dihydropyridine-1(2H)-carboxylate (4, 48 g, 69% yield) as yellow solid. ¹H NMR (400 MHz, DMSO-d₆) δ 10.93 (s, 1H) 7.06-7.14 (m, 2H) 6.98 (d, J=8.1 Hz, 1H) 6.47 (br s, 1H) 4.15 (br s, 2H) 3.93 (br d, J=9.4 Hz, 2H) 3.28 (s, 3H) 1.44 (s, 9H).

### Step 3: tert-butyl 3,3-difluoro-4-(3-methyl-2-oxo-2,3-dihydro-1H-benzo[d]imidazol-5-yl)piperidine-1-carboxylate (5)

tert-butyl 3,3-difluoro-4-(3-methyl-2-oxo-2,3-dihydro-1H-benzo[d]imidazol-5-yl)piperidine-1-carboxylate (5, 45 g, 97% yield) was synthesized from tert-butyl 3,3-difluoro-4-(3-methyl-2-oxo-2,3-dihydro-1H-benzo[d]imidazol-5-yl)-3,6-dihydropyridine-1(2H)-carboxylate (4, 16.4 g, 40.4 mmol) in a similar fashion to Compound B-103, using palladium hydroxide on carbon (16.4 g, 5.84 mmol) and triturating the product with tert-butyl methyl ether. ¹H NMR (400 MHz, CDCl₃) δ 9.47 (br s, 1H) 6.97-7.10 (m, 2H) 6.94 (s, 1H) 4.41 (br s, 2H) 3.44 (s, 3H) 2.73-3.21 (m, 3H) 2.06-2.36 (m, 1H) 1.91 (br d, J=13.3 Hz, 1H) 1.51 (s, 9H); LCMS (ESI−, NH₄CO₃): m/z 366.1 (M−H), rt=1.15 min.

### Step 4: tert-butyl 4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (B-418)

To a solution of tert-butyl 3,3-difluoro-4-(3-methyl-2-oxo-2,3-dihydro-1H-benzo[d]imidazol-5-yl)piperidine-1-carboxylate (5, 21 g, 51.4 mmol) in toluene (600 mL) was added N,N′-dimethylethylenediamine (9.07 g, 103 mmol), K₂CO₃ (21.33 g, 154 mmol), copper (I) iodide (9.80 g, 51.4 mmol) and 2,6-bis(benzyloxy)-3-bromopyridine (6, 85 g, 206 mmol) and the mixture heated to 120° C. for 48 h. Similar reactions were combined and treated to the following workup. The reaction mixture was quenched by addition of water (2000 mL) and extracted with ethyl acetate (3×2000 mL). The combined organic phases were washed with brine (3×500 mL), dried over Na₂SO₄, filtered, concentrated and purified by column chromatography on silica gel (petroleum ether:ethyl acetate 5:1 to 3:1) to give tert-butyl 4-(1-(2,6-bis(benzyloxy)pyridin-3˜yl)-3-methyl-2-oxo-2,3-dihydro-1H-benzo[d]imidazol-5-yl)-3,3-difluoropiperidine-1-carboxylate (B-418, 40 g, 46% yield) as yellow solid. ¹H NMR (400 MHz, DMSO-d₆) δ 7.81 (d, J=8.4 Hz, 1H) 7.31-7.48 (m, 5H) 7.25 (s, 5H) 7.17 (s, 1H) 6.95 (br d, J=7.9 Hz, 1H) 6.58-6.70 (m, 2H) 5.31-5.46 (m, 4H) 4.05-4.38 (m, 2H) 3.39 (s, 4H) 3.28-3.32 (m, 1H) 2.80-3.13 (m, 1H) 2.09 (td, J=12.8, 9.2 Hz, 1H) 1.84 (br d, J=12.6 Hz, 1H) 1.44 (s, 9H). LCMS (ES+): m/z 657.3 [M+H]⁺.

### tert-butyl (4S)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419a) and tert-butyl (4R)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419b)

Configurations are arbitrarily assigned.

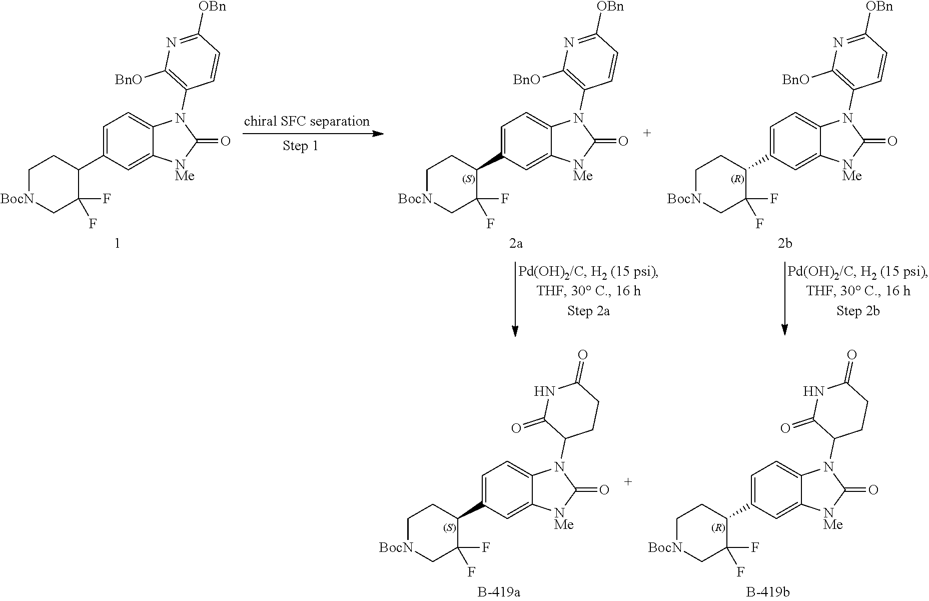

### Step 1: tert-butyl(S)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2a) and tert-butyl (R)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2b)

tert-butyl 4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (1, 40 g, 54.8 mmol) was separated by Chiral SFC using the following conditions: Column: Chiralpak AD-3, 50×4.6 mm I.D., 3 μm. Mobile phase: solvent A: CO₂; solvent B: EtOH (w/0.05% i-PrNH₂, v/v). Flow rate: 3.4 mL/min. Column temperature: 35° C. ABPR: 1800 psi. Gradient (time @ solvent A:B): hold 95:5 from 0.0 to 0.2 min then ramp to 50:50 from 0.2 to 1.2 min then hold 50:50 from 1.2 to 2.2 min then ramp to 95:5 from 2.2 to 2.6 min then hold 95:5 from 2.6 to 3.0 min. The appropriate fractions were pooled and concentrated under reduced pressure to give tert-butyl(S)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2a, rt=1.205 min, 17 g, 45% yield) and tert-butyl (R)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2b, rt=1.267, 15 g, 38% yield) as yellow solid. The first peak is tert-butyl(S)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2a, S-configuration) and the second peak is tert-butyl (R)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2b, R-configuration).

¹H NMR (2a): (400 MHz, DMSO) δ 7.81 (d, J=8.3 Hz, 1H) 7.31-7.48 (m, 5H) 7.25 (s, 5H) 7.16 (s, 1H) 6.95 (d, J=8.1 Hz, 1H) 6.58-6.70 (m, 2H) 5.38 (dd, J=4.5, 3.1 Hz, 4H) 4.00-4.40 (m, 2H) 3.38 (s, 4H) 3.28-3.31 (m, 1H) 2.96 (br s, 1H) 2.08-2.17 (m, 1H) 1.84 (br d, J=12.8 Hz, 1H) 1.44 (s, 9H).

¹H NMR (2b): (400 MHz, DMSO+D₂O) δ 7.81 (br d, J=8.3 Hz, 1H) 7.30-7.49 (m, 5H) 7.25 (s, 5H) 7.17 (s, 1H) 6.94 (br d, J=8.0 Hz, 1H) 6.56-6.71 (m, 2H) 5.30-5.45 (m, 4H) 3.95-4.42 (m, 2H) 3.38 (s, 4H) 3.24-3.32 (m, 1H) 2.97 (br s, 1H) 2.10 (br s, 1H) 1.84 (br d, J=12.5 Hz, 1H) 1.43 (s, 9H).

### Step 2a: tert-butyl (4S)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419a)

To a solution of tert-butyl(S)-4-(1-(2,6-bis(benzyloxy)pyridin-3-yl)-3-methyl-2-oxo-2,3-dihydro-1H-benzo[d]imidazol-5-yl)-3,3-difluoropiperidine-1-carboxylate (2a, 4.5 g, 6.17 mmol) in tetrahydrofuran (90 mL) was added palladium hydroxide on carbon (4.5 g, 1.60 mmol) under Ar. The suspension was degassed and purged with H₂ 3 times and the mixture stirred under H₂ (15 psi) at 30° C. for 16 h. Similar reactions were combined and treated to the following workup. The suspension was filtered through a pad of Celite® and filter cake was washed with tetrahydrofuran (500 mL). The combined filtrates were concentrated, the residue triturated with ethyl acetate (50 mL) and the mixture filtered and dried to give tert-butyl (4S)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419a, 3 g, 87% yield) as white solid.

LCMS (ESI+, TFA): m/z 479.2 (M−1)

¹H NMR (400 MHz, DMSO-d6) δ 11.11 (s, 1H), 7.14 (s, 1H), 7.07 (d, J=8.2 Hz, 1H), 6.97 (br d, J=8.1 Hz, 1H), 5.37 (dd, J=12.8, 5.3 Hz, 1H), 4.04-4.38 (m, 2H), 3.34 (s, 4H), 3.29 (br s, 1H), 2.82-3.10 (m, 2H), 2.56-2.80 (m, 2H), 1.94-2.21 (m, 2H), 1.82 (br d, J=13.0 Hz, 1H), 1.43 (s, 9H)

### Step 2b: tert-butyl (4R)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419b)

tert-butyl (4R)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419b, 4 g, 62% yield) was prepared following the same procedure as tert-butyl (4S)-4-(1-(2,6-dioxopiperidin-3-yl)-3-methyl-2-oxo-2,3-dihydro-1H-benzo[d]imidazol-5-yl)-3,3-difluoropiperidine-1-carboxylate (B-419a).

LCMS (ESI−, NH₄CO₃): m/z 447 (M−H)

¹H NMR (400 MHz, DMSO+D₂O) δ 7.12 (s, 1H), 7.07 (d, J=8.2 Hz, 1H), 6.97 (br d, J=8.1 Hz, 1H), 5.32 (br dd, J=12.8, 5.3 Hz, 1H), 4.02-4.38 (m, 2H), 3.11-3.44 (m, 5H), 2.79-3.09 (m, 2H), 2.57-2.76 (m, 2H), 1.93-2.16 (m, 2H), 1.81 (br d, J=13.1 Hz, 1H), 1.41 (s, 9H).

### 3-(5-((R)-3,3-difluoropiperidin-4-yl)-3-methyl-2-oxo-2,3-dihydro-1H-benzo[d]imidazol-1-yl))piperidine-2,6-dione (B-420)

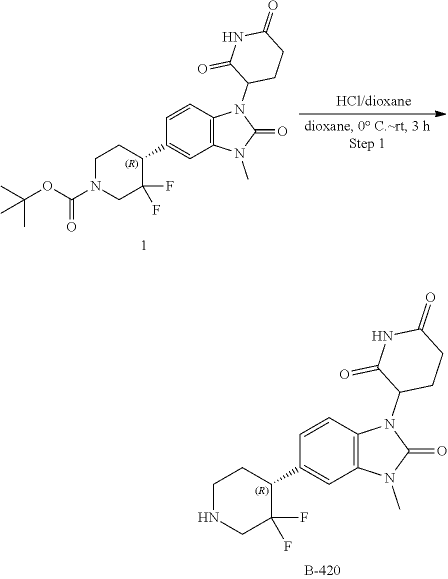

### Step 1: 3-(5-((R)-3,3-difluoropiperidin-4-yl)-3-methyl-2-oxo-2,3-dihydro-1H-benzo[d]imidazol-1-yl)piperidine-2,6-dione (B-420)

To a solution of (4R)-tert-butyl 4-(1-(2,6-dioxopiperidin-3-yl)-3-methyl-2-oxo-2,3-dihydro-1H-benzo[d]imidazol-5-yl)-3,3-difluoropiperidine-1-carboxylate (1, 200 mg, 417.98 μmol) in dioxane (0.5 mL) was added HCl/dioxane (4 M, 5 mL) at 0° C. and stirred at 20° C. for 3 h. The reaction mixture was concentrated under reduced pressure to afford 3-(5-((R)-3,3-difluoropiperidin-4-yl)-3-methyl-2-oxo-2,3-dihydro-1H-benzo[d]imidazol-1-yl)piperidine-2,6-dione (B-420, 180 mg, 416.55 μmol, 99% yield, HCl salt) as a white solid. The material was used in the next step without further purification.

LCMS (ESI): m/z 379.1 [M+H]⁺.

### 3-(5-((S)-3,3-difluoropiperidin-4-yl)-3-methyl-2-oxo-2,3-dihydro-1H-benzo[d]imidazol-1-yl))piperidine-2,6-dione (B-421)

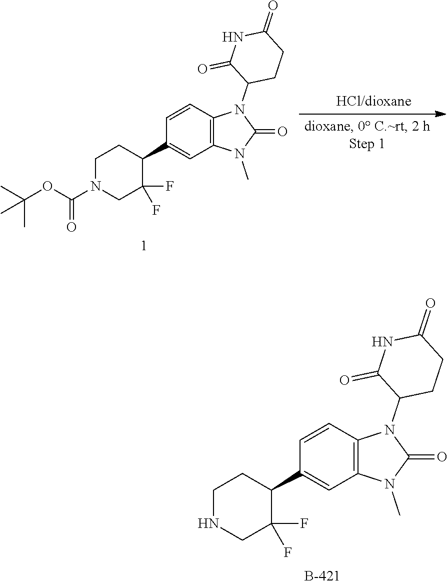 [SMILES: Cn1c(=O)n(C2CCC(=O)NC2=O)c2ccc([C@@H]3CCN(C(=O)OC(C)(C)C)CC3(F)F)cc21 ; Cn1c(=O)n(C2CCC(=O)NC2=O)c2ccc([C@@H]3CCNCC3(F)F)cc21]

### Step 1: 3-(5-((S)-3,3-difluoropiperidin-4-yl)-3-methyl-2-oxo-2,3-dibydro-1H-benzo[d]imidazol-1-yl)piperidine-2,6-dione (B-421)

To a solution of (4S)-tert-butyl 4-(1-(2,6-dioxopiperidin-3-yl)-3-methyl-2-oxo-2,3-dihydro-1H-benzo[d]imidazol-5-yl)-3,3-difluoropiperidine-1-carboxylate (1, 200 mg, 417.98 μmol) in dioxane (2 mL) was added HCl/dioxane (4 M, 4 mL) at 0° C. under N₂. The mixture was stirred at 25° C. for 2 h. The reaction mixture was concentrated under vacuum to afford 3-(5-((S)-3,3-difluoropiperidin-4-yl)-3-methyl-2-oxo-2,3-dihydro-1H-benzo[d]imidazol-1-yl)piperidine-2,6-dione (B-421, 180 mg, 433.91 μmol, HCl salt) as a yellow solid. The material was used in the next step without further purification.

LCMS (ESI): m/z 379.3 [M+H]⁺.

¹H NMR (400 MHz, DMSO-d₆) δ=11.10 (s, 1H), 7.12 (d, J=8.4 Hz, 1H), 7.04 (s, 1H), 6.95 (br d, J=8.0 Hz, 1H), 5.44-5.27 (m, 1H), 3.78 (br t, J=10.8 Hz, 1H), 3.64-3.46 (m, 6H), 3.20-3.09 (m, 1H), 2.98-2.85 (m, 1H), 2.78-2.58 (m, 2H), 2.37-2.21 (m, 1H), 2.13-1.97 (m, 2H).

### 6-bromo-3-(2,6-dibenzyloxy-3-pyridyl)pyrazolo[1,5-a]pyridine (B-422)

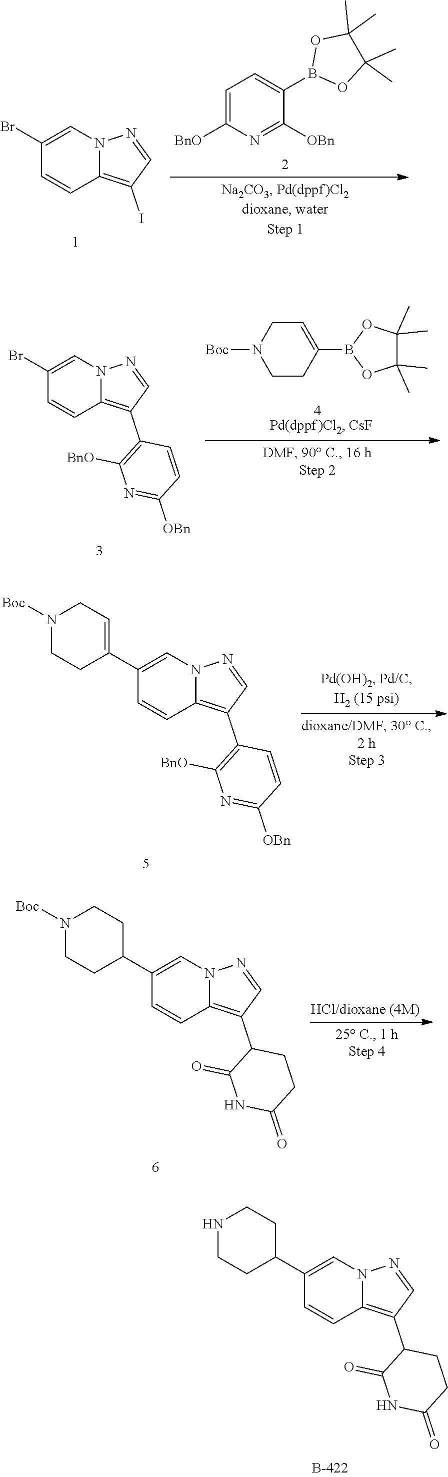

### Step 1: 6-bromo-3-(2,6-dibenzyloxy-3-pyridyl)pyrazolo[1,5-a]pyridine (3)

To a stirred solution of 6-bromo-3-iodo-pyrazolo[1,5-a]pyridine (1, 5 g, 15.48 mmol) and 2,6-dibenzyloxy-3-(4,4,5,5-tetramethyl-1,3,2-dioxaborolan-2-yl)pyridine (2, 9.69 g, 23.22 mmol) in Dioxane (60 mL) and Water (15 mL) was added Sodium carbonate (4.10 g, 38.71 mmol, 1.62 mL) and degassed for 15 min with Argon. Pd(dppf)Cl2·DCM (1.26 g, 1.55 mmol) was added under inert atmosphere. Resulting solution was heated to 90° C. for 16 h. After completion, reaction mixture was cooled to rt, filtered over a short pad of Celite and washed with ethyl acetate. Combined organic part was washed with water and brine. Organic part was separated, dried over anhydrous Na₂SO₄, filtered, concentrated and purified by column chromatography to afford 6-bromo-3-(2,6-dibenzyloxy-3-pyridyl)pyrazolo[1,5-a]pyridine (3, 2.5 g, 5.01 mmol, 32% yield)

1H NMR (400 MHz, DMSO-d6) d 9.06 (s, 1H), 8.20 (s, 1H), 7.85 (d, J=8.0 Hz, 1H), 7.65 (d, J=9.4 Hz, 1H), 7.47-7.27 (m, 11H), 6.57 (d, J-8.0 Hz, 1H), 5.42 (s, 2H), 5.40 (s, 2H).

### Step 2: tert-butyl 4-(3-(2,6-bis(benzyloxy)pyridin-3-yl)pyrazolo[1,5-a]pyridin-6-yl)-5,6-dihydropyridine-1(2H)-carboxylate (5)

tert-butyl 4-(3-(2,6-bis(benzyloxy)pyridin-3-yl)pyrazolo[1,5-a]pyridin-6-yl)-5,6-dihydropyridine-1(2H)-carboxylate (5, 300 mg, 494.31 μmol, 80% yield) was synthesized from 3-(2,6-bis(benzyloxy)pyridin-3-yl)-6-bromopyrazolo[1,5-a]pyridine (3, 300 mg, 616.83 μmol) and tert-butyl 4-(4,4,5,5-tetramethyl-1,3,2-dioxaborolan-2-yl)-5,6-dihydropyridine-1(2H)-carboxylate (4, 247.95 mg, 801.88 μmol) in a similar fashion to Compound B-133, except using 0.1 eq. Pd(dppf)Cl₂.

LCMS (ESI): m/z 589.3 [M+H]⁺.

¹H NMR (400 MHz, CDCl₃) δ 8.40 (s, 1H), 8.15 (s, 1H), 7.71 (d, J=8.4 Hz, 1H), 7.45-7.30 (m, 11H), 7.20-7.18 (m, 1H), 6.51 (d, J=8.0 Hz, 1H), 6.11 (s, 1H), 5.45 (s, 2H), 5.40 (s, 2H), 4.15-4.10 (m, 2H), 3.67 (t, J=2.0 Hz, 2H), 2.52 (s, 2H), 1.50 (s, 9H).

### Step 3: tert-butyl 4-(3-(2,6-dioxopiperidin-3-yl)pyrazolo[1,5-a]pyridin-6-yl)piperidine-1-carboxylate (6)

tert-butyl 4-(3-(2,6-dioxopiperidin-3-yl)pyrazolo[1,5-a]pyridin-6-yl)piperidine-1-carboxylate (6, 80 mg, 147.39 μmol, 36% yield, TFA salt) was synthesized from tert-butyl 4-(3-(2,6-bis(benzyloxy)pyridin-3-yl)pyrazolo[1,5-a]pyridin-6-yl)-5,6-dihydropyridine-1(2H)-carboxylate (5, 240 mg, 407.68 μmol) in a similar fashion to (4, step 2 of Compound B-391, except using Pd/C (20 mg) and Pd(OH)₂ (20 mg) and stirring at 30° C. for 2 h under H₂ (15 psi). The material was purified by MPLC (flow: 60 mL/min; gradient: from 5-50% MeCN in water (0.05% TFA) over 25 min; column: I.D.57 mm×H235 mm, Welch Ultimate XB_C1820-40 μm; 120 Å).

LCMS (ESD): m/z 357 [M−^{t}Bu+H]⁺

¹H NMR (400 MHz, d₆-DMSO) δ 10.84 (s, 1H), 8.46 (s, 1H), 7.83 (s, 1H), 7.56 (d, J=9.2 Hz, 1H), 7.19 (dd, J=1.6, 9.2 Hz, 1H), 4.16-4.08 (m, 3H), 2.75-2.70 (m, 4H), 2.67-2.59 (m, 1H), 2.33-2.28 (m, 1H), 2.11-2.05 (m, 1H), 1.82-1.79 (m, 2H), 1.60-1.50 (m, 2H), 1.42 (s, 9H).

### Step 4: 3-(6-(piperidin-4-yl)pyrazolo[1,5-a]pyridin-3-yl)piperidine-2,6-dione (B-422)

A mixture of tert-butyl 4-(3-(2,6-dioxopiperidin-3-yl)pyrazolo[1,5-a]pyridin-6-yl)piperidine-1-carboxy late (6, 60 mg, 145.46 μmol) in HCl/dioxane (4 M, 4.00 mL) was stirred at 30° C. for 1 h. The reaction mixture was concentrated under reduced pressure to afford 3-(6-(piperidin-4-yl)pyrazolo[1,5-a]pyridin-3-yl)piperidine-2,6-dione (B-422, 50 mg, 143.34 μmol, 99% yield, HCl salt). LCMS (ESI): m/z 313 [M+H]⁺.

### 2-[4-[1-(2,6-dioxo-3-piperidyl)-3-methyl-2-oxo-benzimidazol-5-yl]pyrazol-1-yl]acetic acid (B-423)

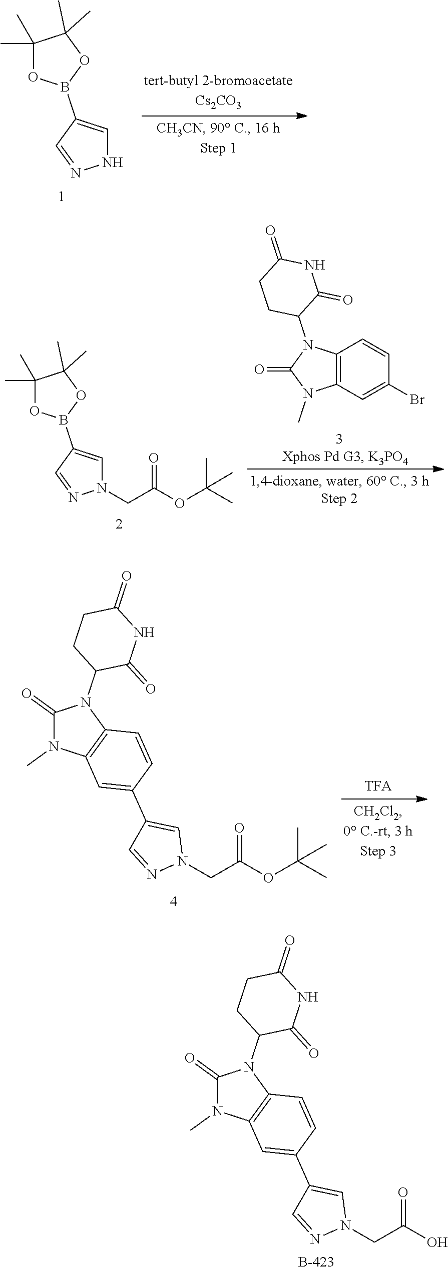 [SMILES: CC1(C)OB(c2cn[nH]c2)OC1(C)C ; CC(C)(C)OC(=O)Cn1cc(B2OC(C)(C)C(C)(C)O2)cn1 ; Cn1c(=O)n(C2CCC(=O)NC2=O)c2ccc(Br)cc21 ; Cn1c(=O)n(C2CCC(=O)NC2=O)c2ccc(-c3cnn(CC(=O)OC(C)(C)C)c3)cc21 ; Cn1c(=O)n(C2CCC(=O)NC2=O)c2ccc(-c3cnn(CC(=O)O)c3)cc21]

### Step 1: tert-butyl 2-[4-(4,4,5,5-tetramethyl-1,3,2-dioxaborolan-2-yl)pyrazol-1-yl]acetate (2)

Into a 25 mL single neck round bottom flask containing a well-stirred solution of 4-(4,4,5,5-tetramethyl-1,3,2-dioxaborolan-2-yl)-1H-pyrazole (1, 4 g, 20.61 mmol) in anhydrous CH₃CN (40 mL) was added Cs₂CO₃ (10.07 g, 30.92 mmol) followed by tert-butyl 2-bromoacetate (4.02 g, 20.61 mmol, 3.02 mL). The mixture was stirred for 16 h at 90° C. The reaction mixture was diluted with water (50 mL) and extracted with EtOAc (2×100 mL). The combined organic layers were dried over Na₂SO₄, filtered and concentrated to afford tert-butyl 2-[4-(4,4,5,5-tetramethyl-1,3,2-dioxaborolan-2-yl)pyrazol-1-yl]acetate (2, 4 g, 8.94 mmol, 43% yield) as a yellow oil. The material was used in the next step without further purification. LCMS (ES+): m/z 309.2 [M+H]⁺.

### Step 2: tert-butyl 2-[4-[1-(2,6-dioxo-3-piperidyl)-3-methyl-2-oxo-benzimidazol-5-yl]pyrazol-1-yl]acetate (4)

Into a 100 mL sealed tube containing a well-stirred solution of 3-(5-bromo-3-methyl-2-oxo-benzimidazol-1-yl)piperidine-2,6-dione (3, 400 mg, 1.18 mmol) and tert-butyl 2-[4-(4,4,5,5-tetramethyl-1,3,2-dioxaborolan-2-yl)pyrazol-1-yl]acetate (2, 528.32 mg, 1.18 mmol) in 1,4-dioxane (8 mL) and water (0.8 mL) was added K₃PO₄ (753.27 mg, 3.55 mmol) and the resulting contents were degassed by with nitrogen gas for 10 min. XPhos Pd G3 (150.19 mg, 177.43 μmol) was added to the reaction mixture and heated at 60° C. for 3 h. The reaction mixture was concentrated to dryness and purified by flash silica-gel (230-400 mesh) column chromatography (85% EtOAc in pet. ether) to afford tert-butyl 2-[4-[1-(2,6-dioxo-3-piperidyl)-3-methyl-2-oxo-benzimidazol-5-yl]pyrazol-1-yl]acetate (4, 400 mg, 800.98 μmol, 68% yield) as an off-white solid. LCMS (ES+): m/z 440.2 [M+H]⁺.

### Step 3: 2-[4-[1-(2,6-dioxo-3-piperidyl)-3-methyl-2-oxo-benzimidazol-5-yl]pyrazol-1-yl]acetic acid (B-423)

2-[4-[1-(2,6-dioxo-3-piperidyl)-3-methyl-2-oxo-benzimidazol-5-yl]pyrazol-1-yl]acetic acid (B-423, 700 mg, 1.17 mmol, 97% yield, TFA salt) was synthesized from tert-butyl 2-[4-[1-(2,6-dioxo-3-piperidyl)-3-methyl-2-oxo-benzimidazol-5-yl]pyrazol-1-yl]acetate (4, 600 mg, 1.20 mmol) in a similar fashion to Compound A-26, except using 29 eq. TFA and triturating the product with diethyl ether. LCMS (ES+): m/z 384.2 [M+H]⁺.

### 3-[6-(4-piperidyl)pyrido[2,3-b]indol-9-yl]piperidine-2,6-dione (B-424)

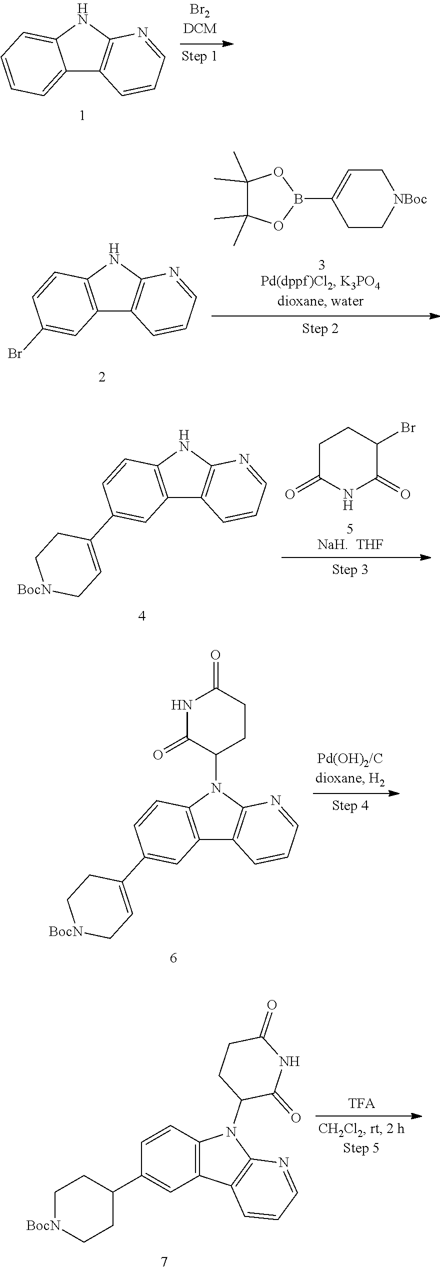

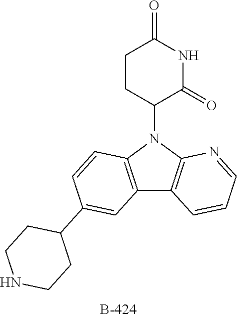 [SMILES: O=C1CCC(n2c3ccc(C4CCNCC4)cc3c3cccnc32)C(=O)N1]

### Step 1: 6-bromo-9H-pyrido[2,3-b]indole (2)

To a stirred solution of 9H-pyrido[2,3-b]indole (1, 5.0 g, 29.73 mmol) in DCM (100 mL) was added molecular bromine (5.70 g, 35.67 mmol) and the reaction mixture was stirred for 30 min at 0° C. After completion of reaction, reaction mixture was filtered and Solid was washed with Ethyl Acetate (2×20 ml) and the solid was basified with Saturated NaHCO₃ and extracted with ethyl acetate (2×100 mL) to afford 6-bromo-9H-pyrido[2,3-b]indole (2, 4 g, 10.95 mmol, 37% yield, 68% purity). LCMS (ES+): m/z 249.0 [M+2H]⁺.

### Step 2: tert-butyl 4-(9H-pyrido[2,3-b]indol-6-yl)-3,6-dihydro-2H-pyridine-1-carboxylate (4)

Into a 50 mL sealed tube containing a well-stirred solution of a mixture of 6-bromo-9H-pyrido[2,3-b]indole (2, 1.0 g, 4.05 mmol) and tert-butyl 4-(4,4,5,5-tetramethyl-1,3-dioxolan-2-yl)-3,6-dihydro-2H-pyridine-1-carboxylate (3, 1.51 g, 4.86 mmol) in water (4 mL) and Dioxane (16 mL) was added Potassium phosphate tribasic anhydrous (2.58 g, 12.14 mmol). The reaction mixture was degassed with nitrogen gas for 10 min and then cyclopentyl(diphenyl)phosphane; dichloromethane; dichloropalladium; iron (330.50 mg, 404.71 μmol) was added and the reaction heated at 100° C. for 16 h. The reaction mixture was cooled rt and filtered through Celite, washed with Ethyl acetate (150 mL). The filtrate was extracted with water (25 mL). The combined organic layer was dried over anhydrous sodium sulfate, concentrated and purified by column chromatography 100-200 silica gel and 30% Ethyl acetate and Hexane used as an eluent to afford tert-butyl 4-(9H-pyrido[2,3-b]indol-6-yl)-3,6-dihydro-2H-pyridine-1-carboxy late (4, 600 mg, 1.54 mmol, 38% yield) as a yellow solid.

LCMS (ES⁺): m/z 350.2 [M+H]⁺.

### Step 3: tert-butyl 4-[9-(2,6-dioxo-3-piperidyl)pyrido[2,3-b]indol-6-yl]-3,6-dihydro-2H-pyridine-1-carboxylate (6)

To a stirred solution of tert-butyl 4-(9H-pyrido[2,3-b]indol-6-yl)-3,6-dihydro-2H-pyridine-1-carboxylate (4, 600 mg, 1.72 mmol) in dry THF (10 mL) was added Sodium hydride (60% dispersion in mineral oil) (197.38 mg, 8.59 mmol) at 0° C. After 5 min, 3-bromopiperidine-2,6-dione (5, 824.26 mg, 4.29 mmol) was added. The mixture was stirred for 16 h at rt. The reaction mixture was cooled to 0° C. and quenched with water (10 mL), immediately extracted with Ethyl acetate (50×2 mL). The organic layers were dried over sodium sulfate, concentrated and purified by column chromatography to afford tert-butyl 4-[9-(2,6-dioxo-3-piperidyl)pyrido[2,3-b]indol-6-yl]-3,6-dihydro-2H-pyridine-1-carboxylate (6, 500 mg, 898.98 μmol, 52% yield).

LCMS (ES+): m/z 461.2 [M+H]⁺.

### Step 4: tert-butyl 4-[9-(2,6-dioxo-3-piperidyl)pyrido[2,3-b]indol-6-yl]piperidine-1-carboxylate (7)

tert-butyl 4-[9-(2,6-dioxo-3-piperidyl)pyrido[2,3-b]indol-6-yl]piperidine-1-carboxylate (7, 450 mg, 786.09 μmol, 91% yield) was synthesized from tert-butyl 4-[9-(2,6-dioxo-3-piperidyl)pyrido[2,3-b]indol-6-yl]-3,6-dihydro-2H-pyridine-1-carboxylate (6, 400 mg, 868.58 μmol) in a similar fashion to Compound B-103, except using 20 wt. % palladium hydroxide on carbon, 50% water (400 mg, 2.85 mmol) and triturating the product with hexane. LCMS (ES+): m/z 463.4 [M+H]⁺.

### Step 5: 3-[6-(4-piperidyl)pyrido[2,3-b]indol-9-yl]piperidine-2,6-dione (B-424)

3-[6-(4-piperidyl)pyrido[2,3-b]indol-9-yl]piperidine-2,6-dione (B-424, 170 mg, 289.01 μmol, 65% yield, TFA salt) was synthesized from tert-butyl 4-(9-(2,6-dioxopiperidin-3-yl)-9H-pyrido[2,3-b]indol-6-yl)piperidine-1-carboxylate (7, 207.5 mg, 446.37 μmol) in a similar fashion to Compound A-62, except using 60 eq. TFA and triturating the product with MTBE. LCMS (ES+); m/z 363.2 [M+H]⁺.

### 3-(5-(piperidin-4-yl)-1H-indol-1-yl)piperidine-2,6-dione (B-425)

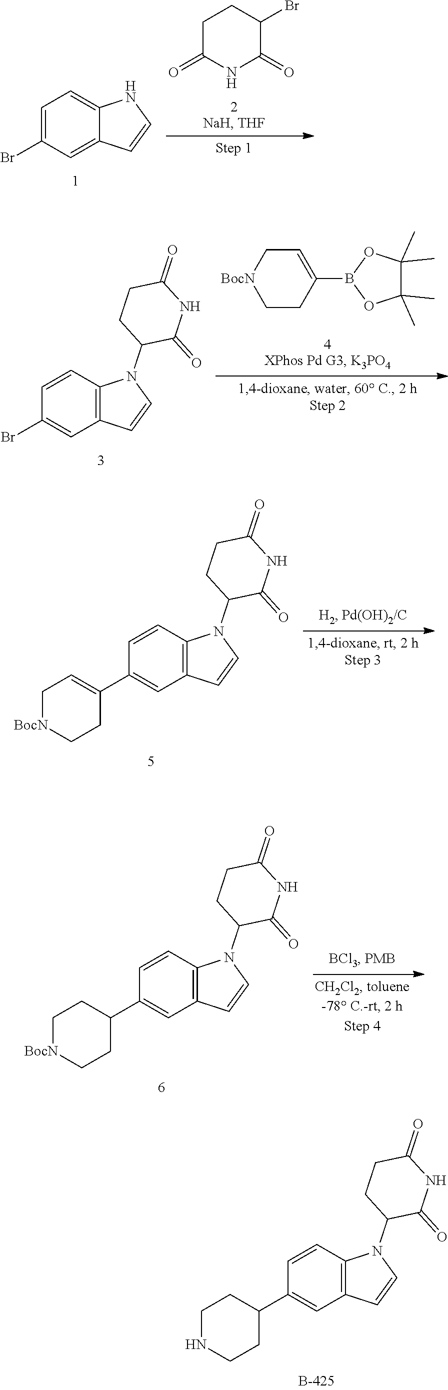

### Step 1: 3-(5-bromoindol-1-yl))piperidine-2,6-dione (3)

Into a 50 mL two neck round bottom flask containing a well-stirred solution of 5-bromo-1H-indole (1, 500 mg, 2.55 mmol) in THF (30 mL) was added Sodium hydride (60% dispersion in mineral oil) (451.04 mg, 12.75 mmol) at 0° C. under nitrogen. The reaction mixture was allowed to stir at ambient temperature for 1 h. 3-bromopiperidine-2,6-dione (2, 832.51 mg, 4.34 mmol) in THF (30 mL) was added at 0° C. The reaction mixture was stirred at 65° C. for 16 h. The reaction mixture was cooled to 0° C., quenched with saturated ammonium chloride solution (20 mL), extracted with ethyl acetate (3×20 mL), the combined organic layer was dried over anhydrous sodium sulfate, filtered and concentrated under reduced pressure to afford 3-(5-bromoindol-1-yl)piperidine-2,6-dione (3, 300 mg, 962.29 μmol, 38% yield) as an off white solid. The material was used in the next step without further purification.

LCMS (ES+): m/z 307.0 [M+H]⁺.

### Step 2: tert-butyl 4-(1-(2,6-dioxopiperidin-3-yl)-1H-indol-5-yl)-3,6-dihydropyridine-1(2H)-carboxylate (5)

Into a 50 mL sealed tube containing a well-stirred solution of 3-(5-bromoindol-1-yl)piperidine-2,6-dione (3, 280 mg, 911.63 μmol) and tert-butyl 4-(4,4,5,5-tetramethyl-1,3,2-dioxaborolan-2-yl)-3,6-dihydro-2H-pyridine-1-carboxylate (4, 338.26 mg, 1.09 mmol) in 1,4-dioxane (4 mL) and water (1 mL) was added K₃PO₄ (387.02 mg, 1.82 mmol) under inert atmosphere and the resulting contents were degassed by bubbling nitrogen gas for 5 min. Then, XPhos Pd G3 (115.75 mg, 136.74 μmol) was added and reaction mixture was heated to 60° C. for 2 h. The reaction mixture was cooled to rt, concentrated under reduced pressure and purified by silica gel flash column chromatography (100-200 mesh silica gel) 0-100% EtOAc in pet-ether to afford tert-butyl 4-(1-(2,6-dioxopiperidin-3-yl)-1H-indol-5-yl)-3,6-dihydropyridine-1(2H)-carboxylate (5, 300 mg, 704.07 μmol, 77% yield) as an off white solid.

LCMS (ES+): m/z 354.2 [M−tBu+H]⁺.

### Step 3: tert-butyl 4-(1-(2,6-dioxopiperidin-3-yl)-1H-indol-5-yl)piperidine-1-carboxylate (6)

tert-butyl 4-(1-(2,6-dioxopiperidin-3-yl)-1H-indol-5-yl)piperidine-1-carboxylate (6, 130 mg, 312.95 μmol, 51% yield) was synthesized from tert-butyl 4-(1-(2,6-dioxopiperidin-3-yl)-1H-indol-5-yl)-3,6-dihydropyridine-1(2H)-carboxylate (5, 260 mg, 609.56 μmol) in a similar fashion to Compound B-103, except using 20 wt. % palladium hydroxide on carbon, 50% water (130 mg, 925.66 μmol). The material was purified by reverse phase column chromatography (Column: REDISPEP C18-120 g, Mobile Phase A: 0.1% NH₄OAc in water and Mobile Phase B: MeCN). LCMS (ES−): m/z 410.1 [M−H]⁺.

### Step 4: 3-(5-(piperidin-4-yl)-1H-indol-1-yl)piperidine-2,6-dione (B-425)

Into a 25 mL single neck round bottom flask containing a well-stirred solution of tert-butyl 4-(1-(2,6-dioxopiperidin-3-yl)-1H-indol-5-yl)piperidine-1-carboxylate (6, 100 mg, 240.59 μmol) and pentamethylbenzene (35.67 mg, 240.59 μmol) in a mixture of dry DCM (3 mL) and dry toluene (1 mL) was added BCl₃ (1.0 M solution in methylene chloride, 481.17 μmol, 481.17 μL) at −78° C. The reaction mixture was stirred at rt for 2 h, before quenching with 1.0 mL of 5% MeOH in DCM at −78° C. The solvents were evaporated and the residue was triturated with MTBE (3 mL) to afford 3-(5-(piperidin-4-yl)-1H-indol-1-yl)piperidine-2,6-dione (B-425, 80 mg, 205.80 μmol, 86% yield) as an off-white solid. LCMS (ES+): m/z 312.2 [M+H]⁺.

### 3-[3-methyl-2-oxo-5-(4-oxo-1-piperidyl)benzimidazol-1-yl]piperidine-2,6-dione (B-426)

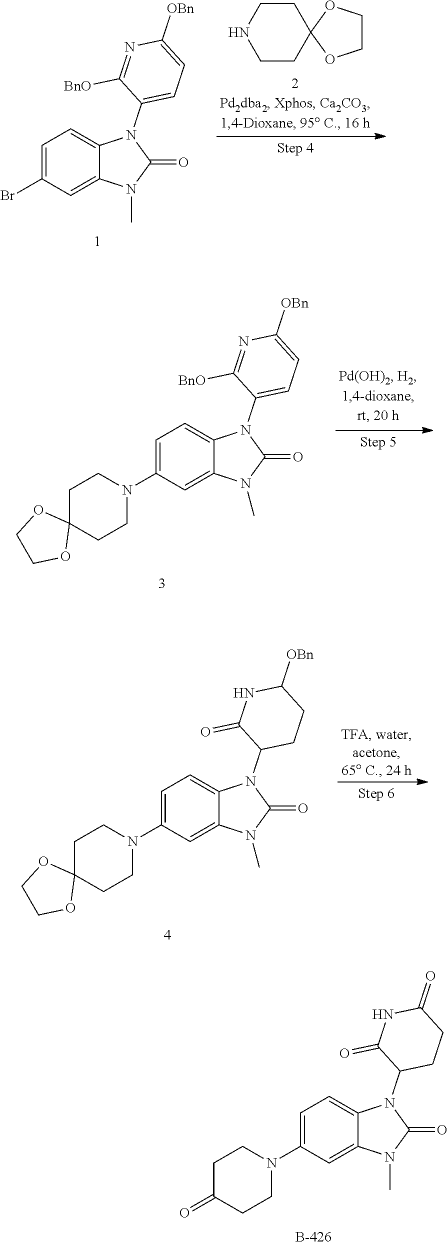 [SMILES: Cn1c(=O)n(-c2ccc(OCc3ccccc3)nc2OCc2ccccc2)c2ccc(Br)cc21 ; C1CC2(CCN1)OCCO2 ; Cn1c(=O)n(-c2ccc(OCc3ccccc3)nc2OCc2ccccc2)c2ccc(N3CCC4(CC3)OCCO4)cc21 ; Cn1c(=O)n(C2CCC(OCc3ccccc3)NC2=O)c2ccc(N3CCC4(CC3)OCCO4)cc21 ; Cn1c(=O)n(C2CCC(=O)NC2=O)c2ccc(N3CCC(=O)CC3)cc21]

### Step 4: 1-(2,6-dibenzyloxy-3-pyridyl)-5-(1,4-dioxa-8-azaspiro[4.5]decan-8-yl)-3-methyl-benzimidazol-2-one (3)

1-(2,6-dibenzyloxy-3-pyridyl)-5-(1,4-dioxa-8-azaspiro[4.5]decan-8-yl)-3-methyl-benzimidazol-2-one (3, 1.4 g, 2.37 mmol, 65% yield) was synthesized from 5-bromo-1-(2,6-dibenzyloxy-3-pyridyl)-3-methyl-benzimidazol-2-one (1, 2 g, 3.64 mmol) and 1,4-dioxa-8-azaspiro[4.5]decane (2, 1.56 g, 10.92 mmol) in a similar fashion to Compound B-180, except using 0.15 eq. XPhos, 0.1 eq. Tris(dibenzylideneacetone) dipalladium (0) at 95° C. LCMS (ES+): m/z 579.2 [M+H]⁺.

### Step 5: 3-[5-(1,4-dioxa-8-azaspiro[4.5]decan-8-yl)-3-methyl-2-oxo-benzimidazol-1-yl]piperidine-2,6-dione (4

3-[5-(1,4-dioxa-8-azaspiro[4.5]decan-8-yl)-3-methyl-2-oxo-benzimidazol-1-yl]piperidine-2,6-dione (4, 0.8 g, 1.99 mmol, 84% yield) was synthesized from 1-(2,6-dibenzyloxy-3-pyridyl)-5-(1,4-dioxa-8-azaspiro[4.5]decan-8-yl)-3-methyl-benzimidazol-2-one (3, 1.4 g, 2.37 mmol) in a similar fashion to Compound B-103, except using 20 wt. % palladium hydroxide on carbon, (1 g, 1.42 mmol). LCMS (ES+): m/z 401.2 [M+H]⁻.

### Step 6: 3-[3-methyl-2-oxo-5-(4-oxo-1-piperidyl)benzimidazol-1-yl]piperidine-2,6-dione (B-426)

Into a 50 mL pressure tube containing a well-stirred solution of 3-[5-(1,4-dioxa-8-azaspiro[4.5]decan-8-yl)-3-methyl-2-oxo-benzimidazol-1-yl]piperidine-2,6-dione (4, 0.8 g, 1.98 mmol) in acetone (4 mL) and water (3 mL) was added TFA (8.88 g, 77.88 mmol, 6 mL) at rt. The reaction mixture was stirred at 65° C. After 16 h, the solvent was removed under reduced pressure and the residue was washed with diethyl ether (30 mL) to afford 3-[3-methyl-2-oxo-5-(4-oxo-1-piperidyl)benzimidazol-1-yl]piperidine-2,6-dione (B-426, 0.7 g, 401.79 μmol, 20% yield, 27% purity, TFA salt) as an off-white solid. The material was taken to next step without purification.

LCMS (ES+): m/z 357.1 [M+H]⁺.

### 3-[6-(3,3-difluoro-4-piperidyl)-1-isopropyl-indazol-3-yl]piperidine-2,6-dione (B-427)

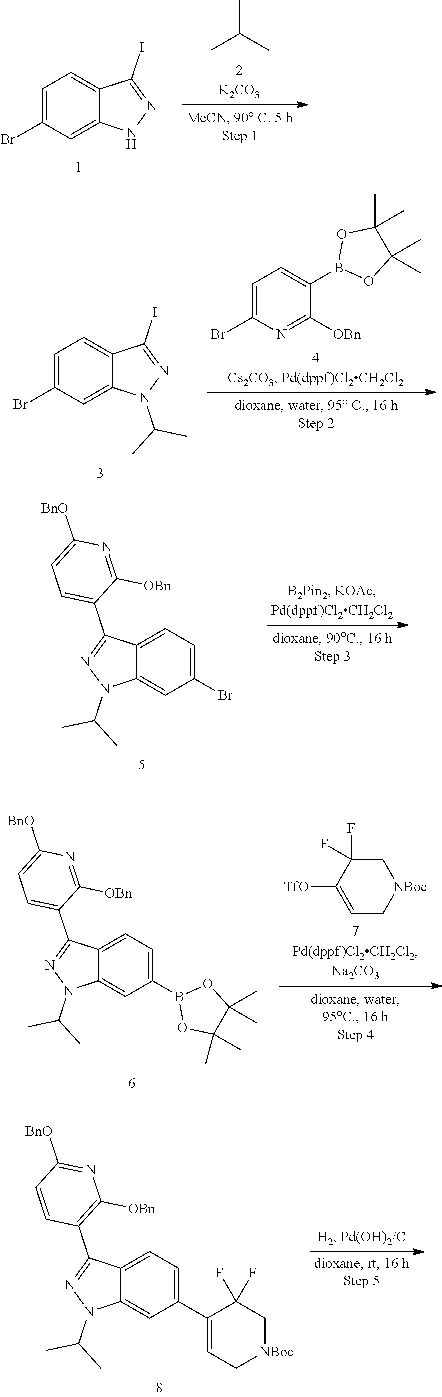

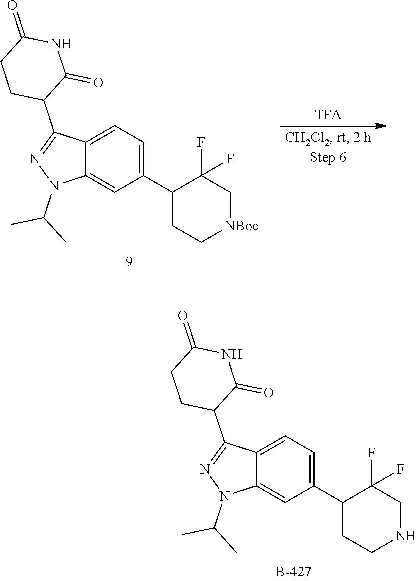 [SMILES: CC(C)n1nc(C2CCC(=O)NC2=O)c2ccc(C3CCN(C(=O)OC(C)(C)C)CC3(F)F)cc21 ; CC(C)n1nc(C2CCC(=O)NC2=O)c2ccc(C3CCNCC3(F)F)cc21]

### Step 1: 6-bromo-3-iodo-1-isopropyl-indazole (3)

Into a 100 mL three-necked round bottom flask containing a well-stirred solution of 6-bromo-3-iodo-1H-indazole (1, 5 g, 15.48 mmol) in anhydrous acetonitrile (100 mL) were added isopropyl iodide (2, 5.26 g, 30.97 mmol, 3.10 mL) and anhydrous potassium carbonate (6.42 g, 46.45 mmol) at ambient temperature under nitrogen atmosphere and the resulting mixture was heated at 90° C. for 5 h. The reaction mixture was poured into water (50 mL) and extracted with EtOAc (2×50 mL). Organic phases were combined and washed with brine (50 mL). Organic layer was dried over anhydrous Na₂SO₄, filtered, concentrated and purified by flash silica-gel (230-400 mesh) chromatography (1% EtOAc in pet ether) to afford a major required regio isomer 6-bromo-3-iodo-1-isopropyl-indazole (3, 3.2 g, 8.30 mmol, 54% yield) as white solid and minor regioisomer 6-bromo-3-iodo-2-isopropyl-indazole (3a, 1.2 g, 3.08 mmol, 20% yield) as white solid.

LCMS (ES+): m/z 365.0 [M+H]⁻.

### Step 2: 6-bromo-3-(2,6-dibenzyloxy-3-pyridyl)-1-isopropyl-indazole (5)

Into a 50 mL sealed tube containing a well-stirred solution of 2,6-dibenzyloxy-3-(4,4,5,5-tetramethyl-1,3,2-dioxaborolan-2-yl)pyridine (4, 851.30 mg, 2.04 mmol) in water (2 mL) were added 6-bromo-3-iodo-1-isopropyl-indazole (3, 745 mg, 2.04 mmol), and cesium carbonate (1.66 g, 5.10 mmol) in 1,4-dioxane (10 mL) at ambient temperature under nitrogen atmosphere. The resulting mixture was degassed by bubbling nitrogen gas into the reaction mixture for 10 min. Subsequently, [1,1′-bis(diphenylphosphino)ferrocene]dichloropalladium(II), complex with dichloromethane (166.46 mg, 204.00 μmol) was added and the resulting reaction mixture was heated to 95° C. for 16 h. The reaction mixture was quenched with water (50 mL) and extracted with EtOAc (2×50 mL). The organic phases were combined and washed with brine, dried (anhydrous Na₂SO₄), filtered, concentrated and purified by flash silica-gel (230-400 mesh) column with (5% EtOAc in pet ether) to afford a product 6-bromo-3-(2,6-dibenzyloxy-3-pyridyl)-1-isopropyl-indazole (5, 650 mg, 1.06 mmol, 52% yield) as a dark syrup.

LCMS (ES+): m/z 528.0 [M+H]⁺

### Step 3: 3-(2,6-dibenzyloxy-3-pyridyl)-1-isopropyl-6-(4,4,5,5-tetramethyl-1,3,2-dioxaborolan-2-yl)indazole (6)

Into a 50 mL sealed tube containing a well-stirred solution of 6-bromo-3-(2,6-dibenzyloxy-3-pyridyl)-1-isopropyl-indazole (5, 650 mg, 934.83 μmol) in anhydrous 1,4-dioxane (10 mL) were added 4,4,5,5-tetramethyl-2-(4,4,5,5-tetramethyl-1,3,2-dioxaborolan-2-yl)-1,3,2-dioxaborolane (356.09 mg, 1.40 mmol), potassium acetate (183.49 mg, 1.87 mmol, 116.87 μL) and the resulting mixture was degassed by bubbling nitrogen gas into the reaction for 10 min. mixture Subsequently, 1,1′-bis(diphenylphosphino)ferrocene]dichloropalladium(II), complex with dichloromethane (76.34 mg, 93.48 μmol) was added and the reaction mixture was heated to 90° C. for 16 h. The reaction mixture was quenched with water (100 mL) and extracted with EtOAc (2×100 mL). The organic phases were combined, washed with brine, dried (anhydrous Na₂SO₄), filtered, concentrated and purified by flash silica-gel (230-400 mesh) column (0-10% EtOAc in pet ether) to afford 3-(2,6-dibenzyloxy-3-pyridyl)-1-isopropyl-6-(4,4,5,5-tetramethyl-1,3,2-dioxaborolan-2-yl) indazole (6, 580 mg, 917.11 μmol, 98% yield) as a syrupy liquid

LCMS (ES+): m/z 576.2 [M+H]⁺.

### Step 4: tert-butyl 4-[3-(2,6-dibenzyloxy-3-pyridyl)-1-isopropyl-indazol-6-yl]-3,3-difluoro-2,6-dihydropyridine-1-carboxylate (8)

Into a 10 mL sealed tube containing a well-stirred solution of 3-(2,6-dibenzyloxy-3-pyridyl)-1-isopropyl-6-(4,4,5,5-tetramethyl-1,3,2-dioxaborolan-2-yl) indazole (6, 100.00 mg, 158.12 μmol) and tert-butyl 3,3-difluoro-4-(trifluoromethylsulfonyloxy)-2,6-dihydropyridine-1-carboxylate (7, 63.13 mg, 158.12 μmol) in dioxane (2 mL) and water (0.2 mL) were added, sodium carbonate (41.90 mg, 395.31 μmol, 16.56 μL) at ambient temperature under nitrogen atmosphere. The resulting mixture was degassed by bubbling nitrogen gas into the reaction mixture for 10 min. Subsequently, 1,1′-bis(diphenylphosphino)ferrocene]dichloropalladium(II), complex with dichloromethane (11.10 mg, 15.81 μmol) was added and the resulting reaction mixture was heated to 95° C. for 16 h. The reaction mixture was quenched with water (20 mL) and extracted with EtOAc (2×30 mL). The organic phases were combined and washed with brine, dried (anhydrous Na₂SO₄), filtered, concentrated and purified by flash silica-gel (230-400 mesh) column (0-10% EtOAc in pet ether) to afford tert-butyl 4-[3-(2,6-dibenzyloxy-3-pyridyl)-1-isopropyl-indazol-6-yl]-3,3-difluoro-2,6-dihydropyridine-1-carboxylate (8, 80 mg, 99.59 μmol, 63% yield) as a thick syrup.

LCMS (ES+): m/z 667.2 [M+H]⁺.

### Step 5: tert-butyl 4-[3-(2,6-dioxo-3-piperidyl)-1-isopropyl-indazol-6-yl]-3,3-difluoro-piperidine-1-carboxylate (9)

tert-butyl 4-[3-(2,6-dioxo-3-piperidyl)-1-isopropyl-indazol-6-yl]-3,3-difluoro-piperidine-1-carboxylate (9, 850 mg, 1.65 mmol, 91% yield) was synthesized from tert-butyl 4-[3-(2,6-dibenzyloxy-3-pyridyl)-1-isopropyl-indazol-6-yl]-3,3-difluoro-2,6-dihydropyridine-1-carboxylate (8, 1.2 g, 1.80 mmol) in a similar fashion to Compound B-103, except using 20 wt. % palladium hydroxide on carbon, 50% water (500 mg, 3.56 mmol) and triturating the product with pet ether. LCMS (ES+): m/z 491.2 [M+H]⁺.

### Step 6: 3-[6-(3,3-difluoro-4-piperidyl)-1-isopropyl-indazol-3-yl]piperidine-2,6-dione (B-427)

3-[6-(3,3-difluoro-4-piperidyl)-1-isopropyl-indazol-3-yl]piperidine-2,6-dione (B-427, 0.8 g, 1.49 mmol, 91% yield, TFA salt) was synthesized from tert-butyl 4-[3-(2,6-dioxo-3-piperidyl)-1-isopropyl-indazol-6-yl]-3,3-difluoro-piperidine-1-carboxylate (9, 850 mg, 162.88 mmol) in a similar fashion to Compound A-62, except using 0.8 eq. TFA and triturating the product with MTBE. LCMS (ES+): m/z 391.2 [M+H]⁺.

### 3-[6-(3,3-difluoro-4-piperidyl)-1-ethyl-indazol-3-yl]piperidine-2,6-dione (B-428)

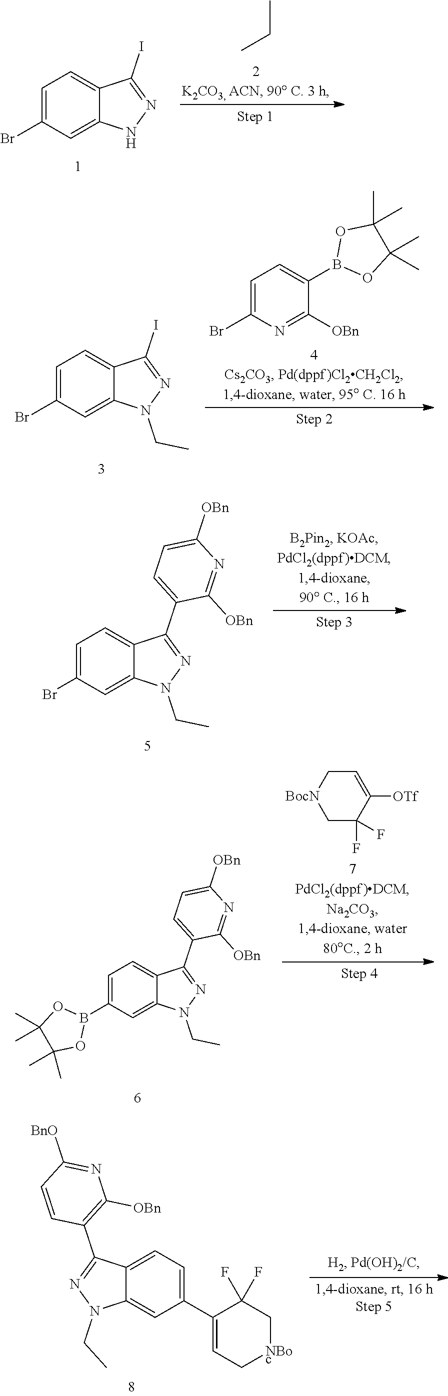

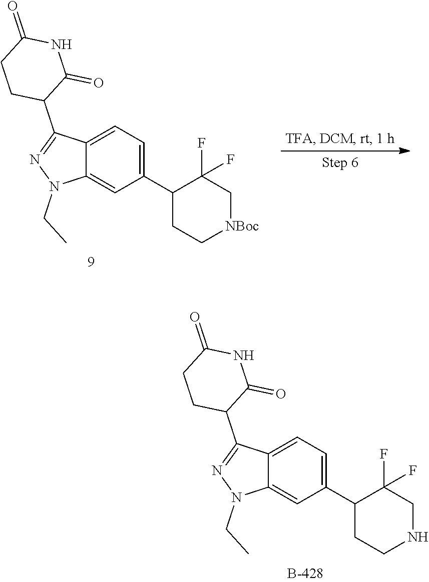 [SMILES: CCn1nc(C2CCC(=O)NC2=O)c2ccc(C3CCN(C(=O)OC(C)(C)C)CC3(F)F)cc21 ; CCn1nc(C2CCC(=O)NC2=O)c2ccc(C3CCNCC3(F)F)cc21]

### Step 1: 6-bromo-1-ethyl-3-iodo-indazole (3)

Into a 250 mL single neck round bottom flask containing a well-stirred solution of 6-bromo-3-iodo-1H-indazole (1, 5 g, 15.48 mmol) in acetonitrile (60 mL) were added anhydrous potassium carbonate (4.28 g, 30.97 mmol) and iodoethane (2, 9.66 g, 61.93 mmol) at rt. The reaction mixture was stirred at 90° C. After 3 h, water (100 mL) was added to the reaction mixture and extracted with EtOAc (3×50 mL). The combined organic layer was dried over anhydrous Na₂SO₄, filtered, concentrated and purified by flash silica gel (230-400 mesh) column chromatography (10% EtOAc in pet ether) to obtain 6-bromo-1-ethyl-3-iodo-indazole (3, 3.88 g, 11.03 mmol, 71% yield) as a colorless solid.

LCMS (ES+): m/z 353.0 [M+H]⁺.

### Step 2: 6-bromo-3-(2,6-dibenzyloxy-3-pyridyl)-1-ethyl-indazole (5)

Into a 250 mL pressure tube containing a well-stirred solution of 6-bromo-1-ethyl-3-iodo-indazole (3, 3.87 g, 11.00 mmol) and 2,6-dibenzyloxy-3-(4,4,5,5-tetramethyl-1,3,2-dioxaborolan-2-yl)pyridine (4, 4.59 g, 11.00 mmol) in 1,4-dioxane (25 mL) and water (2 mL) was added cesium carbonate (7.17 g, 22.00 mmol) at rt. The suspension was degassed by bubbling nitrogen gas for 15 min. Then Pd(dppf)Cl₂·DCM (898.29 mg, 1.10 mmol) was added and stirring was continued at 95° C. The reaction mixture was passed through Celite, washed with ethyl acetate (150 mL) and dioxane (100 mL). The filtrate was concentrated and purified by flash silica gel (230-400 mesh) column chromatography (13% EtOAc in pet-ether) to afford 6-bromo-3-(2,6-dibenzyloxy-3-pyridyl)-1-ethyl-indazole (5, 3.68 g, 6.74 mmol, 61% yield) as an off-white solid.

LCMS (ES+): m/z 514.0 [M+H]⁺.

### Step 3: 3-(2,6-dibenzyloxy-3-pyridyl)-1-ethyl-6-(4,4,5,5-tetramethyl-1,3,2-dioxaborolan-2-yl)indazole (6)

Into a 250 mL pressure tube containing a well-stirred solution of 6-bromo-3-(2,6-dibenzyloxy-3-pyridyl)-1-ethyl-indazole (5, 3.68 g, 6.74 mmol) in 1,4-dioxane (30 mL) were added bis(pinacolato)diboron (1.88 g, 7.41 mmol) and potassium acetate (1.98 g, 20.22 mmol). The mixture was degassed by bubbling nitrogen gas for 15 min. Then Pd(dppf)Cl₂·DCM (550.44 mg, 674.03 μmol) was added and stirred at 90° C. After 16 h, the reaction mixture was passed through Celite and washed with EtOAc (100 mL). The filtrate was concentrated and purified by flash silica gel (230-400 mesh) column chromatography (20% EtOAc in pet ether) to afford 3-(2,6-dibenzyloxy-3-pyridyl)-1-ethyl-6-(4,4,5,5-tetramethyl-1,3,2-dioxaborolan-2-yl) indazole (6, 3.78 g, 5.94 mmol, 88% yield) as gummy off-white solid.

LCMS (ES+): m/z 562.2 [M+H]⁺.

### Step 4: tert-butyl 4-[3-(2,6-dibenzyloxy-3-pyridyl)-1-ethyl-indazol-6-yl]-3,3-difluoro-2,6-dihydropyridine-1-carboxylate (8)

Into a 10 mL pressure tube containing a well-stirred solution of 3-(2,6-dibenzyloxy-3-pyridyl)-1-ethyl-6-(4,4,5,5-tetramethyl-1,3,2-dioxaborolan-2-yl) indazole (6, 156.20 mg, 245.29 μmol) and tert-butyl 3,3-difluoro-4-(trifluoromethylsulfonyloxy)-2,6-dihydropyridine-1-carboxylate (7, 100 mg, 250.48 μmol) in 1,4-dioxane (3 mL) and water (0.3 mL) was added sodium carbonate (64.99 mg, 613.22 μmol). The suspension was degassed by bubbling nitrogen gas for 15 min. Then, Pd(dppf)Cl₂·DCM (20.03 mg, 24.53 μmol) was added and the mixture was stirred at 80° C. After 2 h, the reaction mixture was passed through Celite, washed with ethyl acetate (50 mL) and the filtrate was concentrated and purified by flash silica gel (230-400 mesh) column chromatography (30% EtOAc in pet ether) to afford tert-butyl 4-[3-(2,6-dibenzyloxy-3-pyridyl)-1-ethyl-indazol-6-yl]-3,3-difluoro-2,6-dihydropyridine-1-carboxylate (8, 110 mg, 135.91 μmol, 55% yield) as pale yellow gummy solid.

LCMS (ES+): m/z 653.2 [M+H]⁺.

### Step 5: ter-butyl 4-[3-(2,6-dioxo-3-piperidyl)-1-ethyl-indazol-6-yl]-3,3-difluoro-piperidine-1-carboxylate (9)

Into a 50 mL single-neck round bottom flask containing well-stirred solution of tert-butyl 4-[3-(2,6-dibenzyloxy-3-pyridyl)-1-ethyl-indazol-6-yl]-3,3-difluoro-2,6-dihydropyridine-1-carboxylate (8, 100.00 mg, 123.56 μmol) in anhydrous 1,4-dioxane (5 mL) was added palladium hydroxide on carbon (20 wt. %) (70 mg, 99.69 μmol) at rt. The suspension was stirred at rt under hydrogen atmosphere. After 16 h, the reaction mixture was filtered through Celite and washed with 1,4-dioxane (20 mL). The filtrate was concentrated under reduced pressure and the residue was purified by reverse phase prep HPLC [Purification method: Column: X-Bridge C18 (19×150) mm, 5 micron; Mobile phase A: 0.1% Formic acid in water; Mobile phase B: MeCN] to afford tert-butyl 4-[3-(2,6-dioxo-3-piperidyl)-1-ethyl-indazol-6-yl]-3,3-difluoro-piperidine-1-carboxy late (9, 45 mg, 92.58 μmol, 75% yield) as an off-white solid.

LCMS (ES+): m/z 477.2 [M+H]⁺

### Step 6: 3-[6-(3,3-difluoro-4-piperidyl)-1-ethyl-indazol-3-yl]piperidine-2,6-dione (B-428)

3-[6-(3,3-difluoro-4-piperidyl)-1-ethyl-indazol-3-yl]piperidine-2,6-dione (B-428, 270 mg, 569.24 μmol, 99% yield, Formic acid salt) was synthesized from tert-butyl 4-[3-(2,6-dioxo-3-piperidyl)-1-ethyl-indazol-6-yl]-3,3-difluoro-piperidine-1-carboxylate (550 mg, 577.11 μmol) in a similar fashion to Compound A-62, except using 45 eq. TFA. The material was purified by reverse phase column chromatography (Purification method: Silicycle C18 column; Mobile phase A: 0.1% Formic acid in water; Mobile phase B: MeCN). LCMS (ES+): m/z 377.2 [M+H]⁺.

### 3-[1-(2,6-dioxo-3-piperidyl)-2-oxo-benzo[cd]indol-6-yl]propanal (B-429)

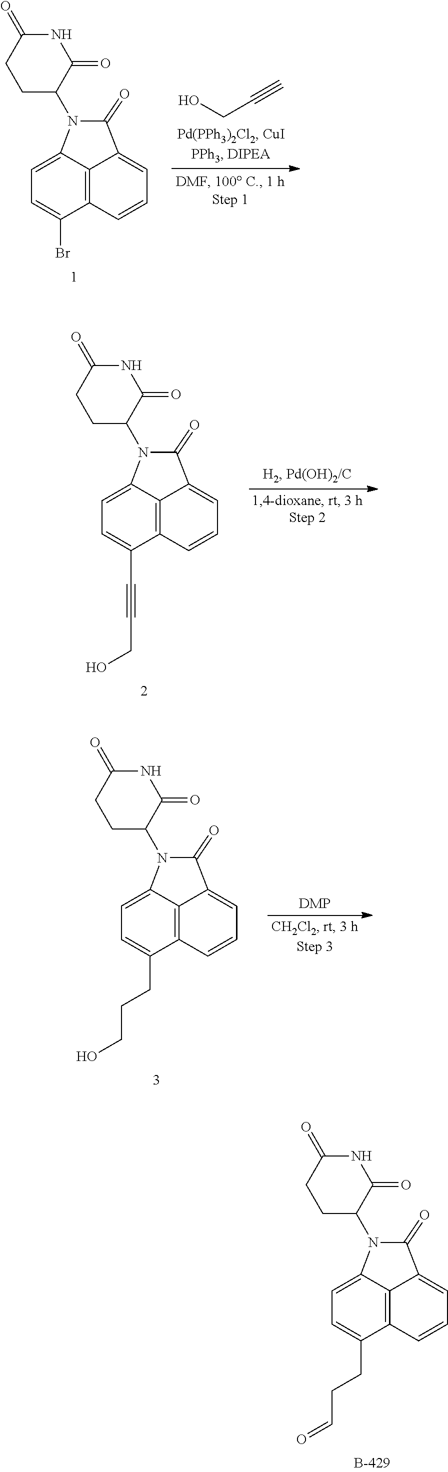

### Step 1: 3-[6-(3-hydroxyprop-1-ynyl)-2-oxo-benzo[cd]indol-1-yl]piperidine-2,6-dione (2)

Into a 100 mL microwave vial containing well-stirred solution of 3-(6-bromo-2-oxo-benzo[cd]indol-1-yl)piperidine-2,6-dione (1, 900 mg, 2.34 mmol) and prop-2-yn-1-ol (262.33 mg, 4.68 mmol, 276.43 μL) in anhydrous DMF (15 mL) were added copper (I) iodide (445.58 mg, 2.34 mmol), triphenyl phosphine (613.66 mg, 2.34 mmol), DIPEA (12.52 g, 96.88 mmol, 16.87 mL) and bis(triphenylphosphine) palladium chloride (164.22 mg, 233.96 μmol). Nitrogen gas was purged through a reaction mixture for 5 min. The reaction mixture was stirred at 100° C. for 1 h under microwave irradiation. The solvent was removed and the residue purified by reverse-phase preparative-HPLC [Column: Redisep gold, 120 g HP C18; Mobile phase A: 0.1% TFA in water and Mobile phase B: MeCN] to afford 3-[6-(3-hydroxyprop-1-ynyl)-2-oxo-benzo[cd]indol-1-yl]piperidine-2,6-dione (2, 600 mg, 1.58 mmol, 67% yield) as a yellow color solid. LCMS (ES−): m/z 333.0 [M−H]⁻

### Step 2: 3-[6-(3-hydroxypropyl)-2-oxo-benzo[cd]indol-1-yl]piperidine-2,6-dione (3)

3-[6-(3-hydroxypropyl)-2-oxo-benzo[cd]indol-1-yl]piperidine-2,6-dione (3, 450 mg, 1.17 mmol, 74% yield) was synthesized from 3-[6-(3-hydroxyprop-1-ynyl)-2-oxo-benzo[cd]indol-1-yl]piperidine-2,6-dione (2, 600 mg, 1.58 mmol) in a similar fashion to Compound B-103, except using 20 wt. % palladium hydroxide on carbon, 50% water (887.09 mg, 1.26 mmol). The material was purified by reverse-phase preparative-HPLC (Column: Redisep gold, 120 g HP C18; Mobile phase A: 0.1% TFA in water and Mobile phase B: MeCN). LCMS (ES−) m/z 337.0 [M−H]⁻.

### Step 3: 3-[1-(2,6-dioxo-3-piperidyl)-2-oxo-benzo[cd]indol-6-yl]propanal (B-429)

Into a 50 mL single neck round bottom flask containing a well-stirred solution of 3-[6-(3-hydroxypropyl)-2-oxo-benzo[cd]indol-1-yl]piperidine-2,6-dione (3, 500 mg, 1.30 mmol) in a 1:1 mixture of THF (5 mL) and DCM (5 mL) was added Dess-Martin periodinane (828.84 mg, 1.95 mmol) under nitrogen atmosphere. After 3 h, the reaction mixture was diluted with saturated sodium thiosulfate (10 mL) and saturated sodium bicarbonate (10 mL). The aqueous layer was extracted with DCM (2×25 mL). The combined organic layers were dried over anhydrous Na₂SO₄, filtered and concentrated under reduced pressure to afford 3-[1-(2,6-dioxo-3-piperidyl)-2-oxo-benzo[cd]indol-6-yl]propanal (B-429, 500 mg, 903.85 μmol, 69% yield, 61% purity) as a yellow solid.

LCMS (ES+): m/z 337.0 [M+H]⁺.

### 3-[3-methyl-2-oxo-5-(3-piperidyl)benzimidazol-1-yl]piperidine-2,6-dione (B-430)

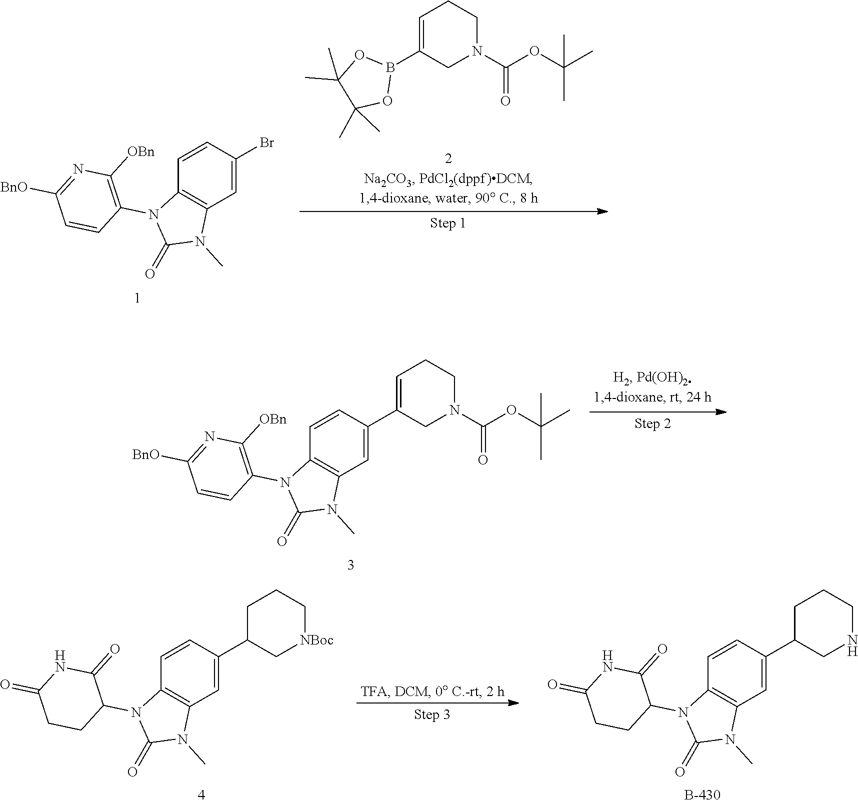 [SMILES: CC(C)(C)OC(=O)N1CCC=C(B2OC(C)(C)C(C)(C)O2)C1 ; Cn1c(=O)n(-c2ccc(OCc3ccccc3)nc2OCc2ccccc2)c2ccc(Br)cc21 ; Cn1c(=O)n(-c2ccc(OCc3ccccc3)nc2OCc2ccccc2)c2ccc(C3=CCCN(C(=O)OC(C)(C)C)C3)cc21 ; Cn1c(=O)n(C2CCC(=O)NC2=O)c2ccc(C3CCCN(C(=O)OC(C)(C)C)C3)cc21 ; Cn1c(=O)n(C2CCC(=O)NC2=O)c2ccc(C3CCCNC3)cc21]

### Step 1: tert-butyl 5-[1-(2,6-dibenzyloxy-3-pyridyl)-3-methyl-2-oxo-benzimidazol-5-yl]-3,6-dihydro-2H-pyridine-1-carboxylate (3)

Into a 100 mL pressure tube containing a well-stirred solution of tert-butyl 5-(4,4,5,5-tetramethyl-1,3,2-dioxaborolan-2-yl)-3,6-dihydro-2H-pyridine-1-carboxylate (2, 1.32 g, 4.26 mmol) and 5-bromo-1-(2,6-dibenzyloxy-3-pyridyl)-3-methyl-benzimidazol-2-one (1, 2 g, 3.87 mmol) in 1,4-dioxane (21 mL) and water (7 mL) was added sodium carbonate (1.23 g, 11.62 mmol). The reaction mixture was degassed by bubbling nitrogen gas for 10 min. Subsequently, Pd(dppf)Cl₂·DCM (158.14 mg, 193.65 μmol) was added and degassed for another 5 min. The reaction mixture was stirred at 90° C. After 8 h, the reaction mixture was diluted with EtOAc (100 mL) and filtered through Celite. The filtrate was dried over sodium sulfate, filtered, concentrated and purified by flash silica gel column chromatography (50% EtOAc in pet ether) to obtain tert-butyl 5-[1-(2,6-dibenzyloxy-3-pyridyl)-3-methyl-2-oxo-benzimidazol-5-yl]-3,6-dihydro-2H-pyridine-1-carboxylate (3, 2.01 g, 3.22 mmol, 83% yield) as a brown sticky solid.

LCMS (ES+): m/z 619.2 [M+H]⁺.

### Step 2: tert-butyl 3-[1-(2,6-dioxo-3-piperidyl)-3-methyl-2-oxo-benzimidazol-5-yl]piperidine-1-carboxylate (4)

tert-butyl 3-[1-(2,6-dioxo-3-piperidyl)-3-methyl-2-oxo-benzimidazol-5-yl]piperidine-1-carboxylate (4, 140 mg, 310.05 μmol, 97% yield) was synthesized from tert-butyl 5-[1-(2,6-dibenzyloxy-3-pyridyl)-3-methyl-2-oxo-benzimidazol-5-yl]-3,6-dihydro-2H-pyridine-1-carboxylate (3, 200 mg, 320.02 μmol) in a similar fashion to Compound B-103, except using 20 wt. % palladium hydroxide on carbon (234.25 mg, 333.61 μmol). LCMS (ES+): m/z 387.2 [M−tBu+H]⁺.

### Step 3: 3-[3-methyl-2-oxo-5-(3-piperidyl)benzimidazol-1-yl]piperidine-2,6-dione (B-430)

3-[3-methyl-2-oxo-5-(3-piperidyl)benzimidazol-1-yl]piperidine-2,6-dione (B-430, 140 mg, 297.54 μmol, 96% yield, TFA salt) was synthesized from tert-butyl 3-[1-(2,6-dioxo-3-piperidyl)-3-methyl-2-oxo-benzimidazol-5-yl]piperidine-1-carboxylate (4, 140 mg, 310.05 μmol) in a similar fashion to Compound A-62, except using 31 eq. TFA. LCMS (ES+): m/z 343.2 [M+H]⁺.

### 2-(4-(1-(2,6-dioxopiperidin-3-yl)-3-isopropyl-2-oxo-2,3-dihydro-1H-benzo[d]imidazol-5-yl)piperidin-1-yl)acetic acid (B-431)

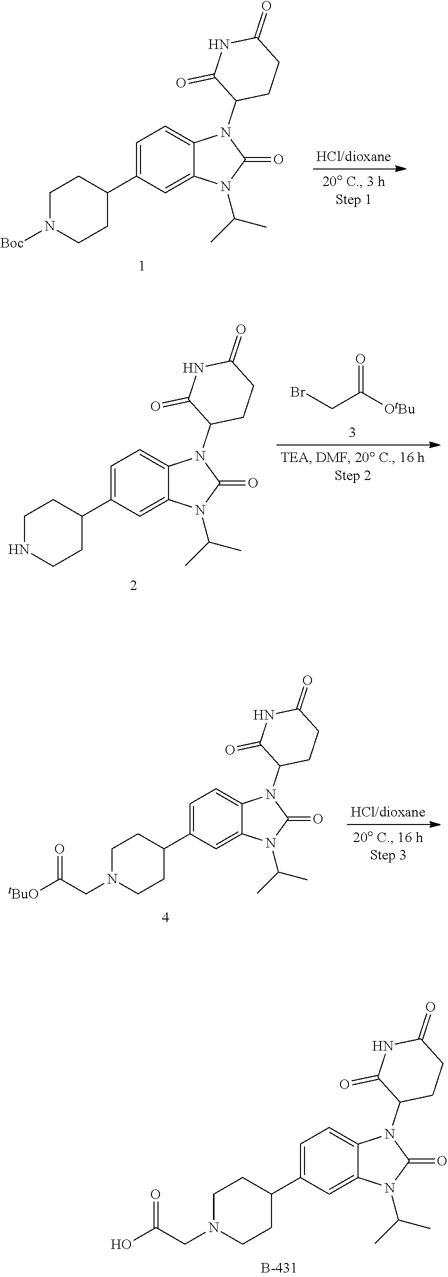 [SMILES: CC(C)n1c(=O)n(C2CCC(=O)NC2=O)c2ccc(C3CCN(C(=O)OC(C)(C)C)CC3)cc21 ; CC(C)n1c(=O)n(C2CCC(=O)NC2=O)c2ccc(C3CCNCC3)cc21 ; CC(C)(C)OC(=O)CBr ; CC(C)n1c(=O)n(C2CCC(=O)NC2=O)c2ccc(C3CCN(CC(=O)OC(C)(C)C)CC3)cc21 ; CC(C)n1c(=O)n(C2CCC(=O)NC2=O)c2ccc(C3CCN(CC(=O)O)CC3)cc21]

### Step 1: 3-(3-isopropyl-2-oxo-5-(piperidin-4-yl)-2,3-dihydro-1H-benzo[d]imidazol-1-yl)piperidine-2,6-dione (2)

3-(3-isopropyl-2-oxo-5-(piperidin-4-yl)-2,3-dihydro-1H-benzo[d]imidazol-1-yl)piperidine-2,6-dione (2 160 mg, 385.35 μmol, 90% yield) was synthesized from tert-butyl 4-(1-(2,6-dioxopiperidin-3-yl)-3-isopropyl-2-oxo-2,3-dihydro-1H-benzo[d]imidazol-5-yl)piperidine-1-carboxylate (1, 200 mg, 425.03 μmol) in a similar fashion to (6, step 3 of Compound A-148/Compound A-149, using HCl/dioxane (2 mL, 4 M) and stirring at 20° C. for 3 h. LCMS (ESI): m/z 371.4 [M+H]⁺.

### Step 2: tert-butyl 2-(4-(1-(2,6-dioxopiperidin-3-yl)-3-isopropyl-2-oxo-2,3-dihydro-1H-benzo[d]imidazol-S-yl)piperidin-1-yl))acetate (4)

To a solution of 3-(3-isopropyl-2-oxo-5-(piperidin-4-yl)-2,3-dihydro-1H-benzo[d]imidazol-1-yl)piperidine-2,6-dione (2, 160 mg, 393.21 μmol) and tert-butyl 2-bromoacetate (3, 230.09 mg, 1.18 mmol, 173.00 μL) in DMF (2 mL) was added TEA (517.26 mg, 5.11 mmol), the mixture was stirred at 20° C. for 16 h. The mixture was concentrated and purified by reversed phase flash (flow: 30 mL/min; gradient: from 40-70% MeCN in water (0.1% TFA) over 18 min; column: Welch Ultimate XB—C18, 20-40 μm, 100 Å, I.D.95 mm×H 365 mm) to afford tert-butyl 2-(4-(1-(2,6-dioxopiperidin-3-yl)-3-isopropyl-2-oxo-2,3-dihydro-1H-benzo[d]imidazol-5-yl)piperidin-1-yl)acetate (4, 180 mg, 371.45 μmol, 60% yield) as a white solid.

LCMS (ESI): m/z 485.3 [M+H]⁺.

### Step 3: 2-(4-(1-(2,6-dioxopiperidin-3-yl)-3-isopropyl-2-oxo-2,3-dihydro-1H-benzo[d]imidazol-5-yl)piperidin-1-yl))acetic acid (B-431)

2-(4-(1-(2,6-dioxopiperidin-3-yl)-3-isopropyl-2-oxo-2,3-dihydro-1H-benzo[d]imidazol-5-yl)piperidin-1-yl)acetic acid (B-431, 26.6 mg, 61.46 μmol, 99% yield) was synthesized from tert-butyl 2-(4-(1-(2,6-dioxopiperidin-3-yl)-3-isopropyl-2-oxo-2,3-dihydro-1H-benzo[d]imidazol-5-yl)piperidin-1-yl)acetate (4, 30.00 mg, 61.91 μmol) in a similar fashion to (6, step 3 of Compound A-148/Compound A-149, using HCl/dioxane (2 mL, 4M) and stirring at 20° C. for 12 h. LCMS (ESI): m/z 429.2 [M+H]⁺.

### 3-(3-(oxetan-3-yl)-2-oxo-5-(piperidin-4-yl)-2,3-dihydro-1H-benzo[d]imidazol-1-yl)piperidine-2,6-dione (B-432)

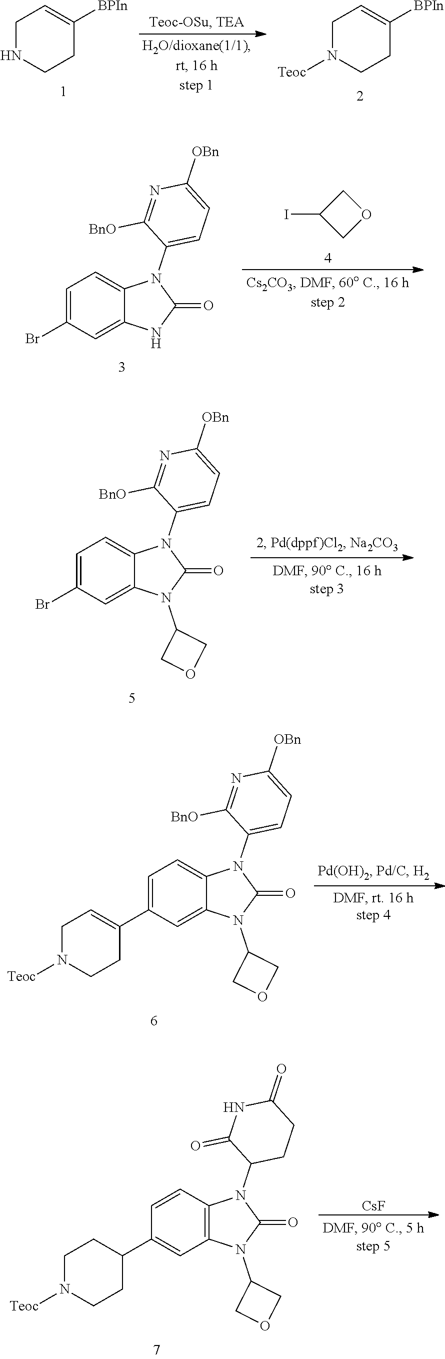

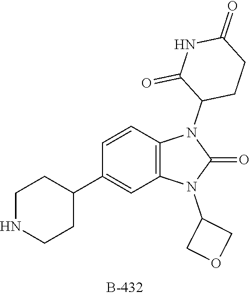 [SMILES: O=C1CCC(n2c(=O)n(C3COC3)c3cc(C4CCNCC4)ccc32)C(=O)N1]

### Step 1: 4-(4,4,5,5-tetramethyl-1,3,2-dioxaborolan-2-yl)-1-(2-(trimethylsilyl))ethoxy)-1,2,3,6-tetrahydropyridine (2)

To a solution of 4-(4,4,5,5-tetramethyl-1,3,2-dioxaborolan-2-yl)-1,2,3,6-tetrahydropyridine (1, 1.5 g, 7.17 mmol, HCl salt) in water (8 mL) was added TEA (3.00 g, 29.65 mmol, 4.13 mL) in dioxane (8 mL). Then Teoc-OSu (3.40 g, 13.12 mmol) was added to the mixture and stirred at 20° C. for 16 h under N2 atmosphere. The reaction mixture was diluted with water (20 mL) and extracted with ethyl acetate (30 mL×3). The combined organic layers were washed with brine (50 mL), dried over Na₂SO₄, filtered and concentrated under reduced pressure to afford 4-(4,4,5,5-tetramethyl-1,3,2-dioxaborolan-2-yl)-1-(2-(trimethylsilyl)ethoxy)-1,2,3,6-tetrahydropyridine (2, 2.2 g, 4.73 mmol, 66% yield) as a yellow solid. The material was used into next step without further purification.

LCMS (ESI): m/z 326.2 [M+H]⁺.

### Step 2:1-(2,6-bis(benzyloxy)pyridin-3-yl)-5-bromo-3-(oxetan-3-yl)-1H-benzo[d]imidazol-2(3H)-one (5)

To a solution of Cs₂CO₃ (1.62 g, 4.98 mmol) in DMF (10 mL) was added 1-(2,6-bis(benzyloxy)pyridin-3-yl)-5-bromo-1H-benzo[d]imidazol-2(3H)-one (3, 1 g, 1.99 mmol) and 3-iodooxetane (4, 732.44 mg, 3.98 mmol). The mixture was stirred at 60° C. for 16 h. The reaction mixture was diluted with water (20 mL) and extracted with ethyl acetate (20 mL×3). The combined organic layers were washed with brine (50 mL×2), dried over Na₂SO₄, filtered and concentrated under reduced pressure to give residue. The residue was purified by flash silica gel chromatography (60 mL/min, Eluent of 0-80% ethyl acetate/petroleum ether gradient, Column: ISCO®; 12 g SepaFlash® Silica Flash Column) to afford 1-(2,6-bis(benzyloxy)pyridin-3-yl)-5-bromo-3-(oxetan-3-yl)-1H-benzo[d]imidazol-2(3H)-one (5, 900 mg, 1.50 mmol, 75% yield) as a yellow oil.

LCMS (ESI): m/z 558.3 [M+H]⁺.

### Step 3: 1-(2,6-bis(benzyloxy)pyridin-3-yl)-3-(oxetan-3-yl)-5-(1-(2-(trimethylsilyl)ethoxy)-1,2,3,6-tetrahydropyridin-4-yl)-1H-benzo[d]imidazol-2(3H)-one (6)

To a solution of 1-(2,6-bis(benzyloxy)pyridin-3-yl)-5-bromo-3-(oxetan-3-yl)-1H-benzo[d]imidazol-2 (3H)-one (5, 550 mg, 984.92 μmol) and 4-(4,4,5,5-tetramethyl-1,3,2-dioxaborolan-2-yl)-1-(2-(trimethylsilyl)ethoxy)-1,2,3,6-tetrahydropyridine (2, 416.54 mg, 1.28 mmol) in DMF (10 mL) were added Pd(dppf)Cl₂ (72.07 mg, 98.49 μmol) and Na₂CO₃ (313.17 mg, 2.95 mmol). The mixture was stirred at 90° C. for 16 h. The reaction mixture was diluted with water (20 mL) and extracted with ethyl acetate (30 mL×3). The combined organic layers were washed with brine (50 mL), dried over Na₂SO₄, filtered and concentrated under reduced pressure to give residue. The residue was purified by flash silica gel chromatography (60 mL/min, Eluent of 0-100% ethyl acetate/petroleum ether gradient, Column: ISCO®; 10 g SepaFlash Silica Flash Column) to afford 1-(2,6-bis(benzyloxy)pyridin-3-yl)-3-(oxetan-3-yl)-5-(1-(2-(trimethylsilyl)ethoxy)-1,2,3,6-tetrahydropyridin-4-yl)-1H-benzo[d]imidazol-2(3H)-one (6, 400 mg, 555.49 μmol, 56% yield) as a yellow oil.

LCMS (ESI): m/z 677.4 [M+H]⁺.

### Step 4: 3-(3-(oxetan-3-yl)-2-oxo-5-(1-(2-(trimethylsilyl)ethoxy)piperidin-4-yl)-2,3-dihydro-1H-benzo[d]imidazol-1-yl)piperidine-2,6-dione (7)

3-(3-(oxetan-3-yl)-2-oxo-5-(1-(2-(trimethylsilyl)ethoxy)piperidin-4-yl)-2,3-dihydro-1H-benzo[d]imidazol-1-yl)piperidine-2,6-dione (7, 200 mg, 395.48 μmol, 67% yield) was synthesized from 1-(2,6-bis(benzyloxy)pyridin-3-yl)-3-(oxetan-3-yl)-5-(1-(2-(trimethylsilyl)ethoxy)-1,2,3,6-tetrahydropyridin-4-yl)-1H-benzo[d]imidazol-2(3H)-one (6, 400 mg, 590.95 μmol) in a similar fashion to (4, step 2 of Compound B-391, except using Pd/C (10%) (150 mg) and Pd(OH)₂/C (10%) (150 mg) and stirring under H₂ (15 Psi) at 20° C. for 16 h. The material was purified by reversed phase flash chromatography (flow: 60 mL/min; gradient: from 10-55% MeCN in water (0.1% TFA) over 20 min; Column: 80 g Flash Column Welch Ultimate XB_C18 20-40 μm; 120 A).

LCMS (ESI): m/z 501.2 [M+H]⁺.

### Step 5: 3-(3-(oxetan-3-yl)-2-oxo-5-(piperidin-4-yl)-2,3-dihydro-1H-benzo[d]imidazol-1-yl)piperidine-2,6-dione (B-432)

To a solution of 3-(3-(oxetan-3-yl)-2-oxo-5-(1-(2-(trimethylsilyl)ethoxy)piperidin-4-yl)-2,3-dihydro-1H-benzo[d]imidazol-1-yl)piperidine-2,6-dione (150 mg, 299.60 μmol) in DMF (1.5 mL) was added CsF (136.53 mg, 898.81 μmol) and stirred at 90° C. for 5 h. The reaction mixture (B-432) was used in next step directly without further purification.

LCMS (ESI): m/z 835.3 [M+H]⁺.

### 3-[4-(3-aminopropyl)-2-oxo-benzo[cd]indol-1-yl]piperidine-2,6-dione (B-433)

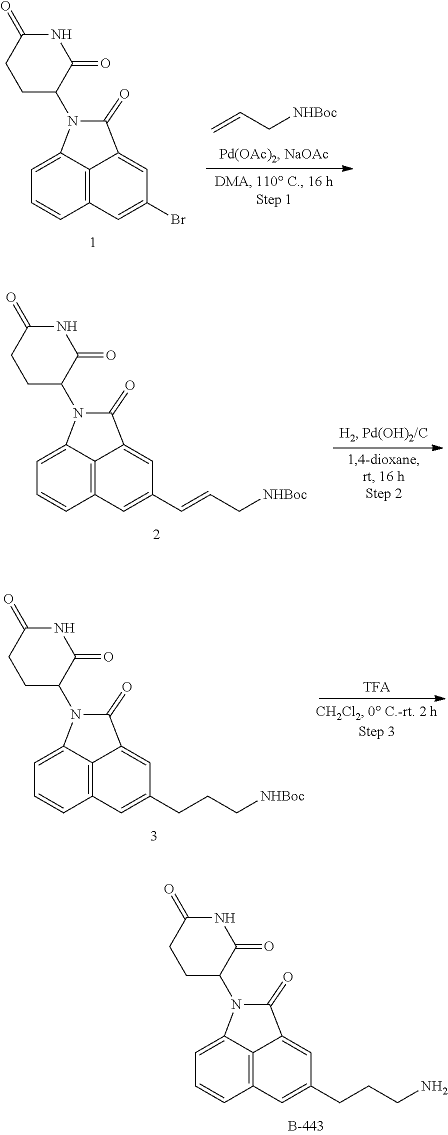 [SMILES: O=C1CCC(N2C(=O)c3cc(Br)cc4cccc2c34)C(=O)N1 ; C=CCNC(=O)OC(C)(C)C ; CC(C)(C)OC(=O)NC/C=C/c1cc2c3c(cccc3c1)N(C1CCC(=O)NC1=O)C2=O ; CC(C)(C)OC(=O)NCCCc1cc2c3c(cccc3c1)N(C1CCC(=O)NC1=O)C2=O ; NCCCc1cc2c3c(cccc3c1)N(C1CCC(=O)NC1=O)C2=O]

### Step 1: tert-butyl (E)-(3-(1-(2,6-dioxopiperidin-3-yl)-2-oxo-1,2-dihydrobenzo[cd]indol-4-yl) allyl) carbamate (2)

Into a 40 mL sealed-tube containing a well-stirred solution of 3-(4-bromo-2-oxobenzo[cd]indol-1(2H)-yl) piperidine-2,6-dione (1.0 g, 2.78 mmol) and tert-butyl allyl carbamate (656.55 mg, 4.18 mmol) in DMA (10 mL) was added anhydrous NaOAc (456.77 mg, 5.57 mmol) and the resulting suspension was degassed with nitrogen for 5 min. Subsequently, palladium(II)acetate (187.52 mg, 835.25 μmol) was added and the mixture was heated at 110° C. for 16 h. The solvent was evaporated and the residue was purified by silica gel flash column chromatography (40-50% EtOAc in pet-ether) to afford tert-butyl (E)-(3-(1-(2,6-dioxopiperidin-3-yl)-2-oxo-1,2-dihydrobenzo[cd]indol-4-yl) allyl) carbamate (2, 400 mg, 677.70 μmol, 24% yield, 74% purity) as pale yellow solid. LCMS (ES+): m/z 380 [M−tBu+H]⁺.

### Step 2: tert-butyl N-[3-[1-(2,6-dioxo-3-piperidyl)-2-oxo-benzo[cd]indol-4-yl]propyl]carbamate (3)

tert-butyl N-[3-[1-(2,6-dioxo-3-piperidyl)-2-oxo-benzo[cd]indol-4-yl]propyl]carbamate (3, 300 mg, 579.79 μmol, 86% yield) was synthesized from tert-butyl (E)-(3-(1-(2,6-dioxopiperidin-3-yl)-2-oxo-1,2-dihydrobenzo[cd]indol-4-yl) allyl) carbamate (2, 400 mg, 677.70 μmol) in a similar fashion to Compound B-103, except using 20 wt. % palladium hydroxide on carbon, 50% water (333.11 mg, 474.39 μmol). LCMS (ES−): m/z 436.3 [M−H]⁻.

### Step 3: 3-[4-(3-aminopropyl)-2-oxo-benzo[cd]indol-1-yl]piperidine-2,6-dione (B-433)

3-[4-(3-aminopropyl)-2-oxo-benzo[cd]indol-1-yl]piperidine-2,6-dione (B-433, 300 mg, 576.28 μmol, 99% yield, TFA salt) was synthesized from tert-butyl N-[3-[1-(2,6-dioxo-3-piperidyl)-2-oxo-benzo[cd]indol-4-yl]propyl]carbamate (3, 300 mg, 579.79 μmol) in a similar fashion to Compound A-62, except using 5 eq. TFA and triturating the product with diethyl ether. LCMS (ES+): m/z 338.2 [M+H]⁺.

### 1-[1-(2,6-dioxo-3-piperidyl)-2-oxo-benzo[cd]indol-5-yl]piperidine-4-carboxylic acid (B-434)

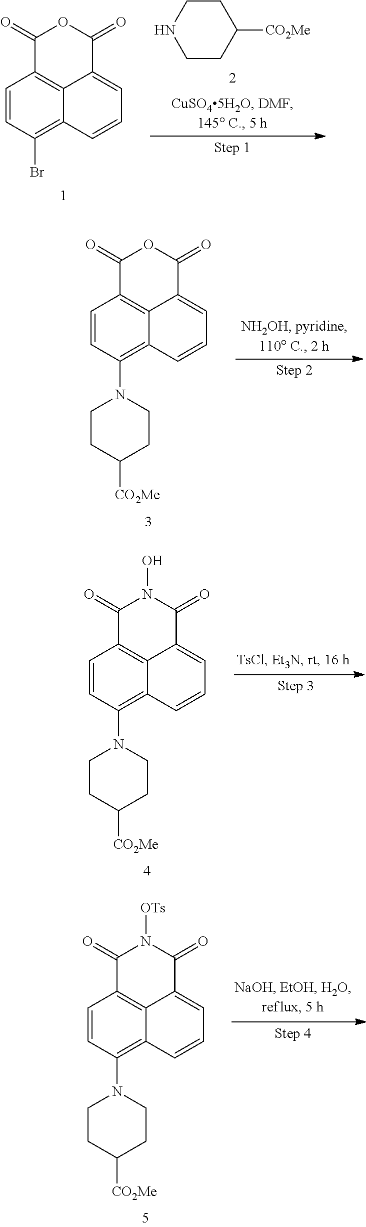 [SMILES: O=C1OC(=O)c2ccc(Br)c3cccc1c23 ; COC(=O)C1CCNCC1 ; COC(=O)C1CCN(c2ccc3c4c(cccc24)C(=O)OC3=O)CC1 ; COC(=O)C1CCN(c2ccc3c4c(cccc24)C(=O)N(O)C3=O)CC1 ; COC(=O)C1CCN(c2ccc3c4c(cccc24)C(=O)N(OS(=O)(=O)c2ccc(C)cc2)C3=O)CC1]

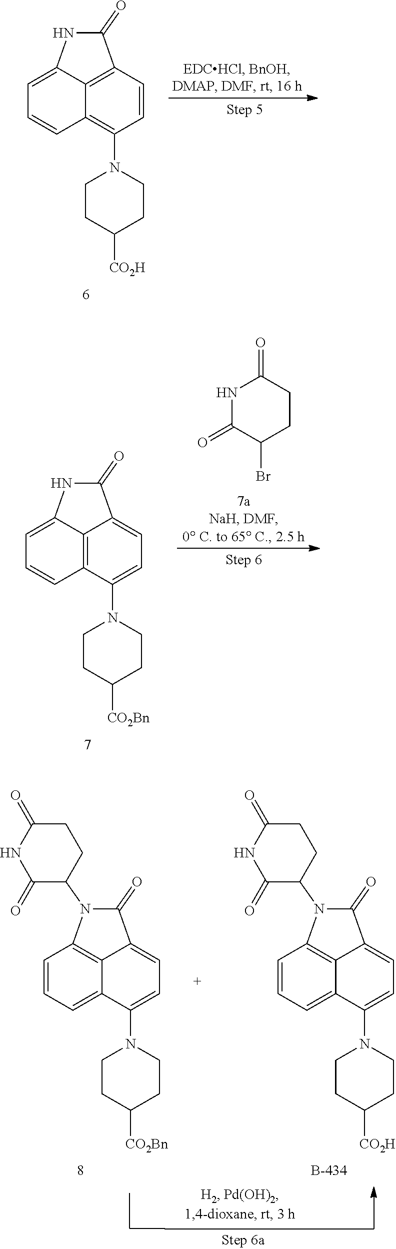 [SMILES: O=C1Nc2cccc3c(N4CCC(C(=O)O)CC4)ccc1c23 ; O=C1Nc2cccc3c(N4CCC(C(=O)OCc5ccccc5)CC4)ccc1c23 ; O=C1CCC(Br)C(=O)N1 ; O=C1CCC(N2C(=O)c3ccc(N4CCC(C(=O)OCc5ccccc5)CC4)c4cccc2c34)C(=O)N1 ; O=C1CCC(N2C(=O)c3ccc(N4CCC(C(=O)O)CC4)c4cccc2c34)C(=O)N1]

### Step 1: methyl 1-(1,3-dioxo-1H,3H-benzo[de]isochromen-6-yl)piperidine-4-carboxylate (3)

Into a 250 mL pressure tube containing a well-stirred solution of 6-bromo-1H,3H-benzo[de]isochromene-1,3-dione (1, 5 g, 18.05 mmol) in DMF (50 mL) were added methyl piperidine-4-carboxylate (2, 3.88 g, 27.07 mmol, 3.66 mL) and copper(II) sulfate pentahydrate (1.35 g, 5.41 mmol). The reaction mixture was stirred at 145° C. After 5 h, the mixture was cooled and diluted with cold water (150 mL). The aqueous phase was extracted EtOAc (3×100 mL). The combined the organic layer was dried over anhydrous Na₂SO₄. The solvent was removed and the residue purified by flash silica gel column chromatography (30% EtOAc in pet-ether) to afford methyl 1-(1,3-dioxo-1H,3H-benzo[de]isochromen-6-yl)piperidine-4-carboxy late (3, 4.2 g, 7.43 mmol, 41% yield, 60% purity) as a yellow solid.

LCMS (ES+): m/z 340.2 [M+H]⁺.

### Step 2: methyl 1-(2-hydroxy-1,3-dioxo-benzo[de]isoquinolin-6-yl)piperidine-4-carboxylate (4)

Into a 250 mL round bottomed flask containing a well-stirred solution of methyl 1-(1,3-dioxo-1H,3H-benzo[de]isochromen-6-yl)piperidine-4-carboxylate (3, 4.2 g, 7.43 mmol) in pyridine (30 mL) was added hydroxylamine hydrochloride (516.05 mg, 7.43 mmol). The reaction mixture was stirred at 110° C. for 2 h. After the completion of reaction, volatiles were evaporated under reduced pressure to afford methyl 1-(2-hydroxy-1,3-dioxo-benzo[de]isoquinolin-6-yl)piperidine-4-carboxylate (4, 3.5 g, 7.39 mmol, 99% yield, 75% purity) as a yellow solid.

LCMS (ES+): m/z 355.2 [M+H]⁺

### Step 3: methyl 1-[1,3-dioxo-2-(p-tolylsulfonyloxy)benzo[de]isoquinolin-6-yl]piperidine-4-carboxylate (5)

Into a 250 mL round bottom round bottom flask containing a well-stirred solution of methyl 1-(2-hydroxy-1,3-dioxo-benzo[de]isoquinolin-6-yl)piperidine-4-carboxy late (4, 3.7 g, 7.81 mmol) in DCM (50 mL) were added triethylamine (1.19 g, 11.71 mmol, 1.63 mL) and 4-methylbenzenesulfonyl chloride (1.79 g, 9.37 mmol). After 5 h, the reaction was quenched with water (60 mL) and extracted with DCM (3×50 mL). The combined organic layer was dried over anhydrous sodium sulfate. The solvent was removed and the residue was purified by flash silica gel column chromatography (50% EtOAc in pet ether) to afford methyl 1-[1,3-dioxo-2-(p-tolylsulfonyloxy)benzo[de]isoquinolin-6-yl]piperidine-4-carboxylate (5, 3.7 g, 6.86 mmol, 88% yield) as a yellow solid.

LCMS: m/z 509.0 [M+H]⁺.

### Step 4:1-(2-oxo-1H-benzo[cd]indol-5-yl)piperidine-4-carboxylic acid (6)

Into a 250 mL 3 neck round bottom flask containing a suspension of methyl 1-[1,3-dioxo-2-(p-tolylsulfonyloxy)benzo[de]isoquinolin-6-yl]piperidine-4-carboxylate (5, 3.7 g, 6.91 mmol) in ethanol (20 mL) and water (10 mL) was added 25 mL of 10% NaOH solution (9.40 g, 235.01 mmol). The resulting mixture was heated at reflux and After 5 h, the mixture was cooled and acidified (pH 5) by adding concentrated HCl dropwise to precipitate a solid that was filtered, washed with cold water and dried to afford 1-(2-oxo-1H-benzo[cd]indol-6-yl)piperidine-4-carboxylic acid (not shown, 1.7 g, 3.44 mmol, 50% yield, HCl salt) as a brown solid. The filtrate was concentrated and the residue was purified by reverse phase prep HPLC [Purification method: Column: X-Select, C18 (150×10) mm, 5 micron; Mobile phase A: 0.1% TFA in water and Mobile phase B: MeCN] to afford 1-(2-oxo-1H-benzo[cd]indol-5-yl)piperidine-4-carboxylic acid (6, 0.25 g, 585.24 μmol, 8% yield, HCl salt) as a brown solid.

LCMS: m/z 297.2 [M+H]⁺.

### Step 5: benzyl 1-(2-oxo-1H-benzo[cd]indol-6-yl)piperidine-4-carboxylate (7)

Into a 25 mL single neck round bottom flask containing well-stirred solution of 1-(2-oxo-1H-benzo[cd]indol-5-yl)piperidine-4-carboxylic acid (6, 0.25 g, 809.94 μmol, HCl salt) in anhydrous DMF (7 mL) were added EDC·HCl (377.20 mg, 2.43 mmol) and DMAP (494.75 mg, 4.05 mmol) at rt. The resulting mixture was stirred at rt for 1 h. Then phenylmethanol (175.17 mg, 1.62 mmol) was added and the reaction mixture was stirred at rt. After 16 h, the solvent was removed under reduced pressure and purified by flash silica gel column chromatography (50% ethyl acetate in pet-ether) to obtain benzyl 1-(2-oxo-1H-benzo[cd]indol-5-yl)piperidine-4-carboxy late (7, 0.19 g, 429.12 μmol, 53% yield) as a yellow solid.

LCMS (ES+): m/z 387.2 [M+H]⁺.

### Step 6: benzyl 2-[1-[1-(2,6-dioxo-3-piperidyl)-2-oxo-benzo[cd]indol-5-yl]-4-piperidyl]acetate (8)

Into a 25 mL two neck round bottom flask containing a well-stirred suspension of NaH (60% dispersion in mineral oil, 136.47 mg, 3.56 mmol) in THF (8 mL) at 0° C. was added benzyl 2-[1-(2-oxo-1H-benzo[cd]indol-5-yl)-4-piperidyl]acetate (0.18 g, 356.16 μmol) and the resulting mixture was stirred at rt for 30 min. Subsequently, 3-bromopiperidine-2,6-dione (273.55 mg, 1.42 mmol) in THF (8 mL) was added and the reaction was stirred at 65° C. for 2 h. The reaction mixture was cooled to 0° C., quenched with saturated NH₄Cl solution (4 mL) and extracted with EtOAc (2×25 mL). The combined organic layers were dried over sodium sulfate, concentrated and purified by reverse phase column chromatography [Purification method: Column-REDISEP-C18-120 g; Mobile phase A: 0.1% Formic acid in water; Mobile phase B: Acetonitrile) to afford a mixture of benzyl 2-[1-[1-(2,6-dioxo-3-piperidyl)-2-oxo-benzo[cd]indol-5-yl]-4-piperidyl]acetate (8, 0.1 g, 172.01 μmol, 48% yield, formic acid salt) LCMS (ES+): m/z 498.2 [M+H]⁺ and 1-[1-(2,6-dioxo-3-piperidyl)-2-oxo-benzo[cd]indol-5-yl]piperidine-4-carboxylic acid (B-434, 85 mg, 50% yield, formic acid salt)

LCMS (ES+): m/z 408.0 [M+H]⁺ as yellow solids.

### Step 6a: 1-[1-(2,6-dioxo-3-piperidyl)-2-oxo-benzo[cd]indol-5-yl]piperidine-4-carboxylic acid (B-434)

1-[1-(2,6-dioxo-3-piperidyl)-2-oxo-benzo[cd]indol-5-yl]piperidine-4-carboxylic acid (B-434, 14.0 mg, 26.46 μmol, 35% yield, TFA salt) was synthesized from benzyl 1-[1-(2,6-dioxo-3-piperidyl)-2-oxo-benzo[cd]indol-5-yl]piperidine-4-carboxylate (8, 0.04 g, 75.35 μmol) in a similar fashion to Compound B-103, except using 20 wt. % palladium hydroxide on carbon (39.68 mg, 56.52 μmol). The material was purified by reverse phase prep HPLC (Purification method: Column: X-Select, C18 (150×30) mm, 5 micron; Mobile phase A: 0.1% TFA in water and Mobile phase B: MeCN). LCMS (ES+): m/z 408.2 [M+H]⁺.

### 1-[1-(2,6-dioxo-3-piperidyl)-2-oxo-benzo[cd]indol-6-yl]piperidine-4-carboxylic acid (B-435)

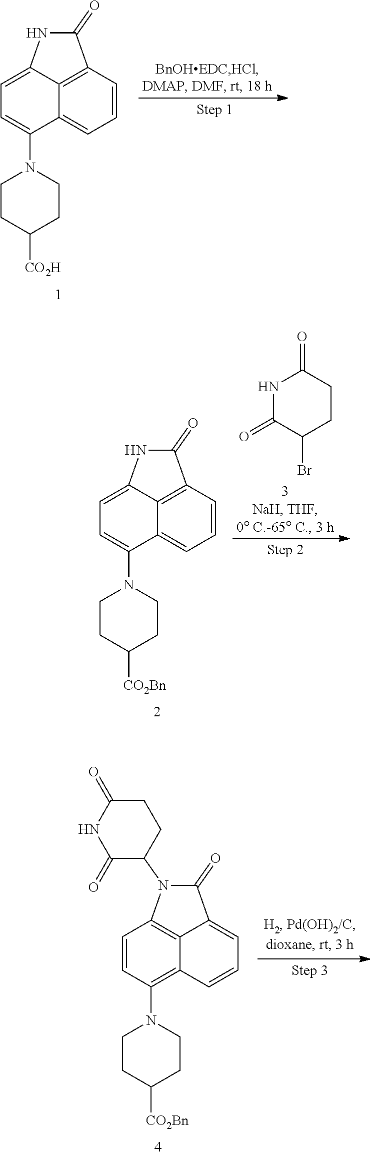 [SMILES: O=C1Nc2ccc(N3CCC(C(=O)O)CC3)c3cccc1c23 ; O=C1Nc2ccc(N3CCC(C(=O)OCc4ccccc4)CC3)c3cccc1c23 ; O=C1CCC(Br)C(=O)N1 ; O=C1CCC(N2C(=O)c3cccc4c(N5CCC(C(=O)OCc6ccccc6)CC5)ccc2c34)C(=O)N1]

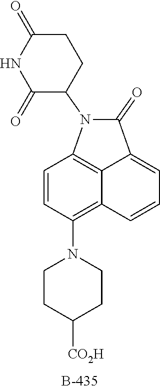 [SMILES: O=C1CCC(N2C(=O)c3cccc4c(N5CCC(C(=O)O)CC5)ccc2c34)C(=O)N1]

### Step 1: benzyl 1-(2-oxo-1H-benzo[cd]indol-6-yl)piperidine-4-carboxylate (2)

To a 50 mL single neck round bottomed flask containing a suspension of 1-(2-oxo-1H-benzo[cd]indol-6-yl)piperidine-4-carboxylic acid (1, 300 mg, 606.81 μmol, TFA salt) in anhydrous DMF (5 mL) were added DMAP (370.67 mg, 3.03 mmol) and EDC·HCl (348.98 mg, 1.82 mmol) and the solution was stirred for 15 min. Then, phenylmethanol (131.24 mg, 1.21 mmol, 124.99 μL) was added and the reaction mixture was stirred at rt. After 18 h, the reaction mixture was concentrated under vacuum and the residue was subjected to flash Silica gel column chromatography (45-50% EtOAc in pet-ether) to obtain benzyl 1-(2-oxo-1H-benzo[cd]indol-6-yl)piperidine-4-carboxylate (2, 275 mg, 604.88 μmol, 99% yield) as a yellow liquid.

LCMS (ES+): m/z 387.2 [M+H]⁺.

### Step 2: benzyl 1-[1-(2,6-dioxo-3-piperidyl)-2-oxo-benzo[cd]indol-6-yl]piperidine-4-carboxylate (4)

To a 100 mL two neck round bottom flask containing a well-stirred solution of benzyl 1-(2-oxo-1H-benzo[cd]indol-6-yl)piperidine-4-carboxy late (2, 275 mg, 604.88 μmol) in THF (22 mL) was added sodium hydride (60% dispersion in mineral oil) (241.93 mg, 6.05 mmol) at 0° C. and the mixture stirred at ambient temperature for 1 h. Then a solution of 3-bromopiperidine-2,6-dione (3, 580.71 mg, 3.02 mmol) in anhydrous THF (5 mL) was added dropwise at 0° C. The reaction mixture was stirred at 65° C. After 2 h. the reaction mixture was quenched with aq. sat. ammonium chloride solution (15 mL) and diluted with water (15 mL) at 0° C. The product was extracted into EtOAc (3×30 mL) and the combined EtOAc layer was washed with brine solution (30 mL), dried over sodium sulfate, filtered, concentrated and purified by flash Silica gel column chromatography (50-55% EtOAc in pet ether) to obtain benzyl 1-[1-(2,6-dioxo-3-piperidyl)-2-oxo-benzo[cd]indol-6-yl]piperidine-4-carboxylate (4, 180 mg, 238.77 μmol, 39% yield, 66% purity) as an orange solid.

LCMS (ES+): m/z 498.2 [M+H]⁺.

### Step 3: 1-[1-(2,6-dioxo-3-piperidyl)-2-oxo-benzo[cd]indol-6-yl]piperidine-4-carboxylic acid (B-435)

1-[1-(2,6-dioxo-3-piperidyl)-2-oxo-benzo[cd]indol-6-yl]piperidine-4-carboxylic acid (B-435, 125 mg, 214.77 μmol, 90% yield, 70% purity) was synthesized from benzyl 1-[1-(2,6-dioxo-3-piperidyl)-2-oxo-benzo[cd]indol-6-yl]piperidine-4-carboxylate (4, 180 mg, 238.77 μmol) in a similar fashion to Compound B-103, except using 20 wt. % palladium hydroxide on carbon (180 mg, 256.35 μmol) and triturating the product with MTBE. LCMS (ES+): m/z 408.2 [M+H]⁺.

### 3-[3-(4-piperidyl)pyrido[2,3-b]indol-9-yl]piperidine-2,6-dione (B-436)

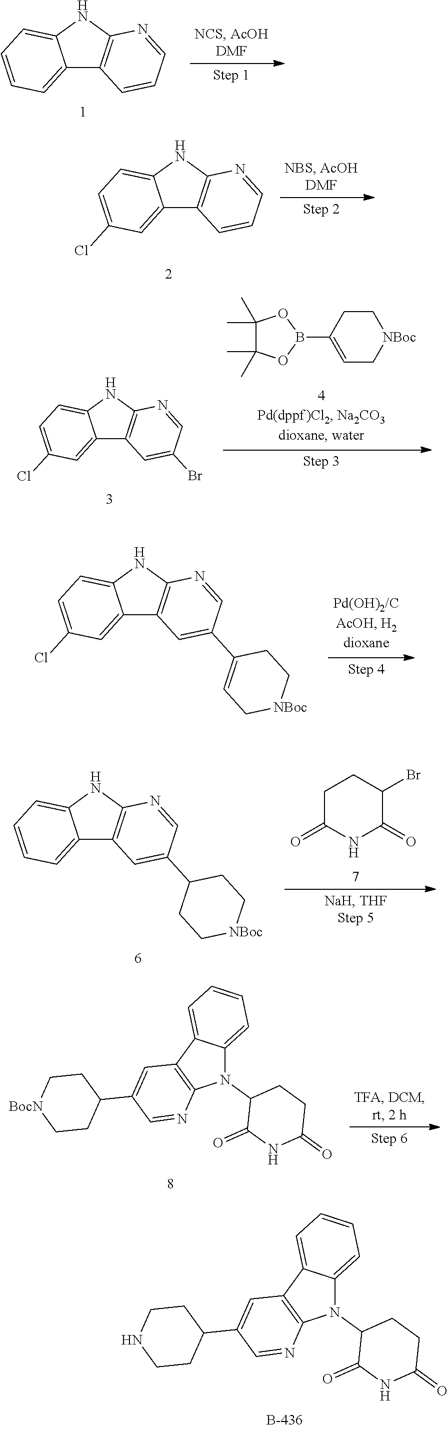

### Step 1: 6-chloro-9H-pyrido[2,3-b]indole (2)

To a stirred solution of 9H-pyrido[2,3-b]indole (1, 10 g, 59.46 mmol) in DMF (200 mL) was added acetic acid (5.35 g, 89.18 mmol, 5.10 mL) and N-Chlorosuccinimide (11.91 g, 89.18 mmol, 7.22 mL) at ambient temperature under nitrogen atmosphere and the resulting mixture was stirred at that temperature for 3 days. Upon completion of the reaction, the reaction mixture was poured into cold water (500 mL), the observed precipitate was filtered, washed with excess water and dried to afford 6-chloro-9H-pyrido[2,3-b]indole (2, 10 g, 38.99 mmol, 66% yield, 79% purity) as an off white solid. The material was used in the next step without further purification.

LCMS (ESI): m/z 203.0 [M+H]⁺.

### Step 2: 3-bromo-6-chloro-9H-pyrido[2,3-b]indole (3)

To a stirred solution of 6-chloro-9H-pyrido[2,3-b]indole (2, 10 g, 49.35 mmol) in DMF (200 mL) was added acetic acid (2.96 g, 49.35 mmol, 2.82 mL) and N-Bromosuccinimide (6.15 g, 34.54 mmol) at ambient temperature under nitrogen atmosphere and the resulting mixture was stirred at that temperature for 16 h. Another portion of N-Bromosuccinimide (0.2 eq.) was added and the reaction was stirred for 4 h. After completion of the reaction as confirmed by LCMS, the reaction mixture was poured into cold water (500 mL), the observed precipitate was filtered, washed with excess water and dried to afford 3-bromo-6-chloro-9H-pyrido[2,3-b]indole (3, 8 g, 22.16 mmol, 45% yield, 78% purity) as a brown solid. The material was used in the next step without further purification.

LCMS (ESI): m/z 280.9 [M+H]⁺.

### Step 3: tert-butyl 4-(6-chloro-9H-pyrido[2,3-b]indol-3-yl)-3,6-dihydro-2H-pyridine-1-carboxylate (4)

Into a 250 mL sealed-tube containing a well-stirred solution of 3-bromo-6-chloro-9H-pyrido[2,3-b]indole (3, 3 g, 10.66 mmol) and tert-butyl 4-(4,4,5,5-tetramethyl-1,3,2-dioxaborolan-2-yl)-3,6-dihydro-2H-pyridine-1-carboxy late (4, 3.29 g, 10.66 mmol) in Water (20 mL) and dioxane (80 mL) was added Sodium carbonate (2.26 g, 21.31 mmol, 892.82 μL) at ambient temperature under nitrogen atmosphere and the resulting mixture was degassed by bubbling nitrogen gas into the reaction mixture for 10 min. Subsequently, [1,1′-Bis(diphenylphosphino)ferrocene]dichloropalladium(II), complex with dichloromethane (435.10 mg, 532.79 μmol) was added to the reaction mixture and the reaction mixture was heated to 90° C. for 16 h. The reaction mixture was cooled to ambient temperature. The reaction mixture was quenched with water (100 ml) and the product was extracted with EtOAc (2×500 ml). The organic phases were combined, dried (anhydrous Na₂SO₄), filtered, concentrated and purified by column chromatography (100 silica-gel column) with 0-100% EtOAc/DCM to afford tert-butyl 4-(6-chloro-9H-pyrido[2,3-b]indol-3-yl)-3,6-dihydro-2H-pyridine-1-carboxylate (5, 2.6 g, 4.94 mmol, 46% yield, 73% purity) as a brown solid.

Product confirmed by LCMS (m/z=384.0), this product taken to next batch.

### Step 4: tert-butyl 4-(9H-pyrido[2,3-b]indol-3-yl)piperidine-1-carboxylate (6)

tert-butyl 4-(9H-pyrido[2,3-b]indol-3-yl)piperidine-1-carboxylate (6, 40 mg, 67.15 μmol, 52% yield, 59% purity) was synthesized from tert-butyl 4-(6-chloro-9H-pyrido[2,3-b]indol-3-yl)-3,6-dihydro-2H-pyridine-1-carboxy late (5, 50 mg, 130.25 μmol) in a similar fashion to Compound B-103, except using 20 wt. % palladium hydroxide on carbon, dry (18.29 mg, 130.25 μmol). LCMS (ESI): m/z: 352.3 [M+H]⁺.

### Step 5: tert-butyl 4-[9-(2,6-dioxo-3-piperidyl)pyrido[2,3-b]indol-3-yl]piperidine-1-carboxylate (8)

To a stirred solution of tert-butyl 4-(9H-pyrido[2,3-b]indol-3-yl)piperidine-1-carboxylate (6, 800 mg, 2.28 mmol) in THF (20 mL) was added Sodium hydride (60% dispersion in mineral oil) (418.66 mg, 17.45 mmol). After 15 min, 3-bromopiperidine-2,6-dione (7, 2.19 g, 11.38 mmol) was added portion wise. After 16 h, excess reagent was quenched with saturated NH₄Cl solution (10 mL). Aqueous layer was extracted with Ethyl acetate (2×50 mL). Organic layers were combined, dried (anhydrous Na₂SO₄), filtered, concentrated and purified by flash silica-gel (230-400 mesh) column with 70-80% EtOAc\pet ether as a eluent to afford the product. This product was re-purified by reverse phase purification: Column: Isco gold 30 g; C8; Mobile phase: A: 0.1% FA:ACN; Flow rate: 15.0 ml/min; to afford tert-butyl 4-[9-(2,6-dioxo-3-piperidyl)pyrido[2,3-b]indol-3-yl]piperidine-1-carboxylate (150 mg, 323.97 μmol, 14% yield) tert-butyl 4-[9-(2,6-dioxo-3-piperidyl)pyrido[2,3-b]indol-3-yl]piperidine-1-carboxylate (8, 150 mg, 323.97 μmol, 14% yield) as a white solid.

LCMS (ESI): m/z 463.4 [M+H]⁺.

### Step 6: 3-[3-(4-piperidyl)pyrido[2,3-b]indol-9-yl]piperidine-2,6-dione (B-436)

3-[3-(4-piperidyl)pyrido[2,3-b]indol-9-yl]piperidine-2,6-dione (B-436, 100 mg, 205.00 μmol, 99% yield, TFA salt) was synthesized from tert-butyl 4-[9-(2,6-dioxo-3-piperidyl)pyrido[2,3-b]indol-3-yl]piperidine-1-carboxylate (8, 95 mg, 205.39 μmol) in a similar fashion to Compound A-62, except using 60 eq. TFA and triturating the product with diethyl ether. LCMS (ES+): m/z 363.2 [M+H]⁺.

### 3-(6-(4-(2-aminoethyl)piperidin-1-yl)-2-oxobenzo[cd]indol-1(2H)-yl)piperidine-2,6-dione (B-437)

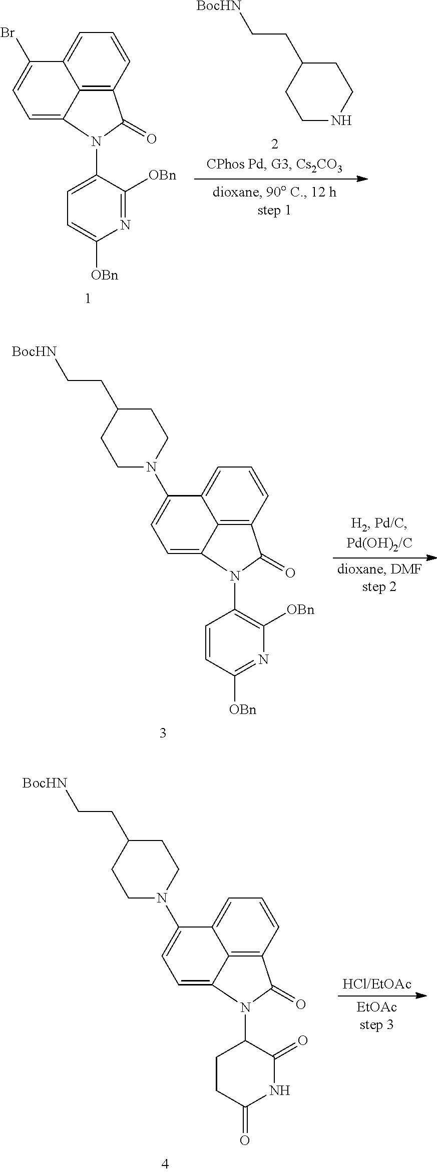 [SMILES: O=C1c2cccc3c(Br)ccc(c23)N1c1ccc(OCc2ccccc2)nc1OCc1ccccc1 ; CC(C)(C)OC(=O)NCCC1CCNCC1 ; CC(C)(C)OC(=O)NCCC1CCN(c2ccc3c4c(cccc24)C(=O)N3c2ccc(OCc3ccccc3)nc2OCc2ccccc2)CC1 ; CC(C)(C)OC(=O)NCCC1CCN(c2ccc3c4c(cccc24)C(=O)N3C2CCC(=O)NC2=O)CC1]

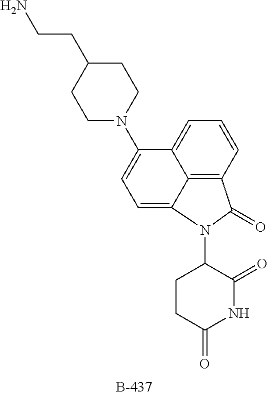 [SMILES: NCCC1CCN(c2ccc3c4c(cccc24)C(=O)N3C2CCC(=O)NC2=O)CC1]

### Step 1: tert-butyl (2-(1-(1-(2,6-bis(benzyloxy)pyridin-3-yl)-2-oxo-1,2-dihydrobenzo[cd]indol-6-yl)piperidin-4-ylethyl) carbamate (3)

To a solution of 1-(2,6-bis(benzyloxy)pyridin-3-yl)-6-bromobenzo[cd]indol-2 (1H)-one (1, 500 mg, 930.40 μmol) and tert-butyl (2-(piperidin-4-yl)ethyl) carbamate (2, 318.66 mg, 1.40 mmol) in dioxane (10 mL) was added dicesium; carbonate (606.28 mg, 1.86 mmol) and CPhos-Pd-G₃ (75.02 mg, 93.04 μmol). The mixture was stirred at 90° C. for 12 h. The reaction mixture was concentrated under reduced pressure to remove dioxane. The residue was purified by column chromatography (SiO₂, Petroleum ether/Ethyl acetate=20/1 to 3/1) to afford tert-butyl (2-(1-(1-(2,6-bis(benzyloxy)pyridin-3-yl)-2-oxo-1,2-dihydrobenzo[cd]indol-6-yl)piperidin-4-yl)ethyl) carbamate (3, 530 mg, 746.06 μmol, 80% yield) as a yellow solid.

LCMS (ESI): m/z 685.6 [M+H]⁺.

### Step 2: tert-butyl (2-(1-(1-(2,6-dioxopiperidin-3-yl)-2-oxo-1,2-dihydrobenzo[cd]indol-6-yl)piperidin-4-ylethyl) carbamate (4)

tert-butyl (2-(1-(1-(2,6-dioxopiperidin-3-yl)-2-oxo-1,2-dihydrobenzo[cd]indol-6-yl)piperidin-4-ylethyl) carbamate (4, 150 mg, 296.10 μmol, 81% yield) was synthesized from tert-butyl (2-(1-(1-(2,6-bis(benzyloxy)pyridin-3-yl)-2-oxo-1,2-dihydrobenzo[cd]indol-6-yl)piperidin-4-yl)ethyl) carbamate (3, 250 mg, 365.06 μmol) in a similar fashion to (4, step 2 of Compound B-391, except using 10 wt. % Pd/C (200 mg, 164.67 μmol) and 10 wt. % Pd(OH)₂/C (200 mg, 365.06 μmol) and stirring under H₂ (15 Psi) at 25° C. for 36 h. The material was purified by column chromatography (SiO₂, Petroleum ether/Ethyl acetate=10/1 to 1/1). LCMS (ESI): m/z 507.5 [M+H]⁺.

### Step 3: 3-(6-(4-(2-aminoethyl)piperidin-1-yl)-2-oxobenzo[cd]indol-1(2H)-yl)piperidine-2,6-dione (B-437)

To a solution of tert-butyl (2-(1-(1-(2,6-dioxopiperidin-3-yl)-2-oxo-1,2-dihydrobenzo[cd]indol-6-yl)piperidin-4-yl)ethyl) carbamate (4, 150 mg, 296.10 μmol) in EtOAc (2 mL) was added HCl/EtOAc (4 M, 2 mL) at 0° C. The mixture was stirred at 20° C. for 2 h. The reaction mixture was concentrated under reduced pressure to remove EtOAc to afford 3-(6-(4-(2-aminoethyl)piperidin-1-yl)-2-oxobenzo[cd]indol-1 (2H)-yl)piperidine-2,6-dione (B-437, 130 mg, 293.50 μmol, 99% yield) as a yellow solid. The material was used in the next step without further purification. LCMS (ESI): m/z 407.5 [M+H]⁺.

### 4-(((1-(2,6-dioxopiperidin-3-yl)-2-oxo-1,2-dibydrobenzo[cd]indol-6-yl)methyl)amino)-4-oxobutanoic acid (B-438)

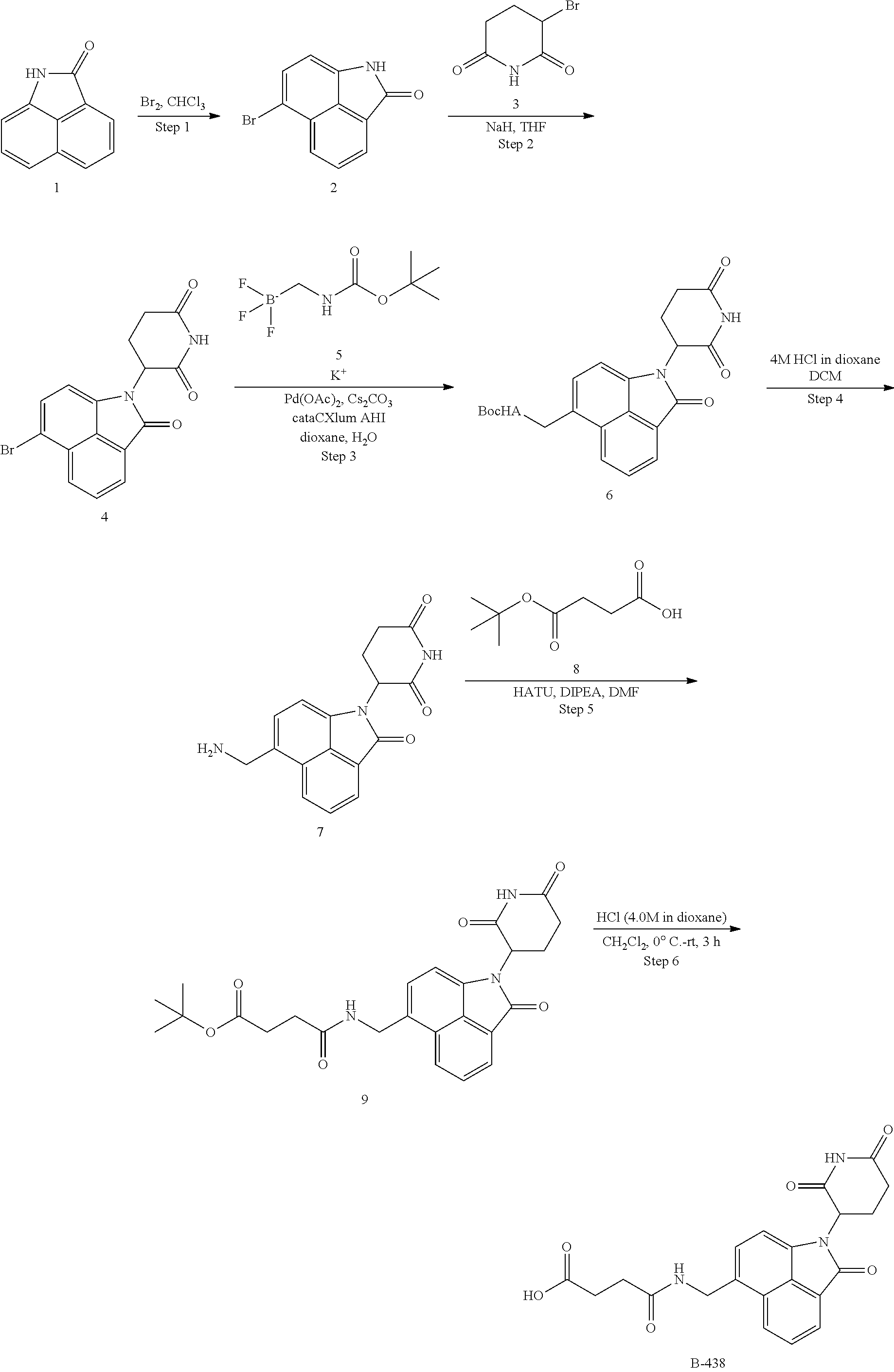

### Step 1: 6-bromo-1H-benzo[cd]indol-2-one (2)

Into a 2 L three-neck round-bottom flask containing a well-stirred solution of 1H-benzo[cd]indol-2-one (1, 20.00 g, 118.22 mmol) in chloroform (1.2 L), cooled to 0° C., was added bromine (14.36 g, 177.52 mmol, 9.64 mL) dropwise at 0° C. The reaction mixture was stirred at rt for 20 h. The reaction was quenched carefully with sodium thiosulfate solution (1000 mL) in cold condition and the resulting yellow solid was filtered. Then the solid was washed with cold water (250 mL) and diethyl ether (150 mL) to afford 6-bromo-1H-benzo[cd]indol-2-one (2, 22.0 g, 82.56 mmol, 70% yield) as a yellow solid. LCMS (ES⁺): m/z 250.0 [M+2]⁺.

### Step 2: 3-(6-bromo-2-oxo-benzo[cd]indol-1-yl)piperidine-2,6-dione (4)

Into a 500 mL three-neck round-bottom flask containing a well-stirred solution of 6-bromo-1H-benzo[cd]indol-2-one (2, 2.50 g, 10.08 mmol) in THF (200 mL), cooled to 0° C., sodium hydride (60% dispersion in mineral oil) (2.00 g, 52.20 mmol) was added and the resulting mixture was stirred rt for 1 h. To this, 3-bromopiperidine-2,6-dione (3, 4.84 g, 25.19 mmol) in THF (10 mL) was added dropwise at 0° C. and the mixture was heated at 60° C. for 16 h. The reaction was slowly quenched at 0° C. with saturated NH₄Cl solution and extracted with ethyl acetate (2×100 mL). The combined organic layer was dried over anhydrous Na₂SO₄, filtered and concentrated under reduced pressure. The product was recrystallized with dichloromethane (10 mL) and dried to afford 3-(6-bromo-2-oxo-benzo[cd]indol-1-yl)piperidine-2,6-dione (4, 2.5 g, 6.31 mmol, 63% yield) as a yellow solid. LCMS (ES⁺): m/z 360.8 [M+H]⁺.

### Step 3: tert-butyl N-[[1-(2,6-dioxo-3-piperidyl)-2-oxo-benzo[cd]indol-6-yl]methyl]carbamate (6)

Into a 250 mL sealed-tube containing a well-stirred solution of 3-(6-bromo-2-oxo-benzo[cd]indol-1-yl)piperidine-2,6-dione (4, 2.00 g, 5.57 mmol) and potassium [[(tert-butoxycarbonyl)amino]methyl]trifluoroborate (5, 3.30 g, 13.92 mmol) in 1,4-dioxane (60 mL), were added cesium carbonate (5.44 g, 16.71 mmol) and water (8 mL). The reaction mixture was degassed with N₂ for 10 min before palladium(II)acetate (125.01 mg, 556.83 μmol) and di(1-adamantyl)-n-butylphosphine (99.82 mg, 278.42 μmol) were added. The mixture was degassed with nitrogen for 5 min and was heated to 100° C. for 16 h. The mixture was cooled to 0° C., slowly quenched with saturated NH₄Cl solution and extracted with ethyl acetate (2×75 mL). The combined organic layer was dried over anhydrous Na₂SO₄, filtered and concentrated under reduced pressure. The product was purified by flash column chromatography (Silica gel, 230-400 mesh) using 0-100% ethyl acetate in pet ether to afford tert-butyl N-[1-(2,6-dioxo-3-piperidyl)-2-oxo-benzo[cd]indol-6-yl]methyl]carbamate (6, 900 mg, 1.78 mmol, 32% yield) as a yellow solid. LCMS (ES+): m/z 293.1 [M-117+H]⁺.

### Step 4: 3-[6-(aminomethyl)-2-oxo-benzo[cd]indol-1-yl]piperidine-2,6-dione (7)

Into a 100 mL single neck round bottom flask containing a well-stirred solution of tert-butyl N-[[1-(2,6-dioxo-3-piperidyl)-2-oxo-benzo[cd]indol-6-yl]methyl]carbamate (6, 1.4 g, 3.42 mmol) in anhydrous DCM (10 mL), was added 4.0 M hydrogen chloride solution in dioxane (5 mL) at 0° C. The resulting mixture was stirred at rt for 1 h. The solvent was removed and the residue triturated with diethyl ether (10 mL) and dried to afford 3-[6-(aminomethyl)-2-oxo-benzo[cd]indol-1-yl]piperidine-2,6-dione (7, 1.2 g, 2.95 mmol, 86% yield, HCl salt) as an yellow solid.

### Step 5: tert-butyl 4-[[1-(2,6-dioxo-3-piperidyl)-2-oxo-benzo[cd]indol-6-yl]methylamino]-4-oxo-butanoate (9)

Into a 50 mL single neck round bottom flask containing a well-stirred solution of 3-[6-(aminomethyl)-2-oxo-benzo[cd]indol-1-yl]piperidine-2,6-dione (7, 600 mg, 1.94 mmol) and 4-tert-butoxy-4-oxo-butanoic acid (8, 337.89 mg, 1.94 mmol) in DMF (5 mL). The mixture was cooled to 0° C. and DIPEA (752.08 mg, 5.82 mmol, 1.01 mL) and HATU (1.11 g, 2.91 mmol) were added and the mixture was stirred at rt for 1 h. The mixture was cooled to 0° C., quenched with ice cold water (30 mL) and extracted with ethyl acetate (2×50 mL). The combined organic layer was dried over anhydrous Na₂SO₄, filtered and concentrated under reduced pressure. The material was purified by flash column chromatography (Silica gel, 230-400 mesh) using 0-100% ethyl acetate in pet ether to afford tert-butyl 4-[[1-(2,6-dioxo-3-piperidyl)-2-oxo-benzo[cd]indol-6-yl]methylamino]-4-oxo-butanoate (9, 1.0 g, 1.80 mmol, 93% yield) as a yellow liquid. LCMS (ES⁺): m/z 410.0 [M-56+H]⁺.

### Step 6: 4-(((1-(2,6-dioxopiperidin-3-yl)-2-oxo-1,2-dihydrobenzo[cd]indol-6-yl)methyl)amino)-4-oxobutanoic acid (B-438)

Into a 100 mL single neck round bottom flask containing a well-stirred solution of tert-butyl 4-[[1-(2,6-dioxo-3-piperidyl)-2-oxo-benzo[cd]indol-6-yl]methylamino]-4-oxo-butanoate (9, 700 mg, 1.35 mmol) in anhydrous DCM (10 mL) was added HCl (4.0 M in dioxane, 33.83 mmol, 8.5 mL) dropwise at 0° C. The reaction mixture was stirred at rt for 3 h. The reaction mixture was concentrated under reduced pressure and triturated with MTBE to obtain 4-(((1-(2,6-dioxopiperidin-3-yl)-2-oxo-1,2-dihydrobenzo[cd]indol-6-yl)methyl)amino)-4-oxobutanoic acid (B-438, 555 mg, 1.20 mmol, 89% yield) as a yellow solid. LCMS (ES−): m/z 408.0 [M−H]⁻.

### 3-[3-methyl-2-oxo-5-(piperazin-1-ylmethyl)benzimidazol-1-yl]piperidine-2,6-dione (B-439)

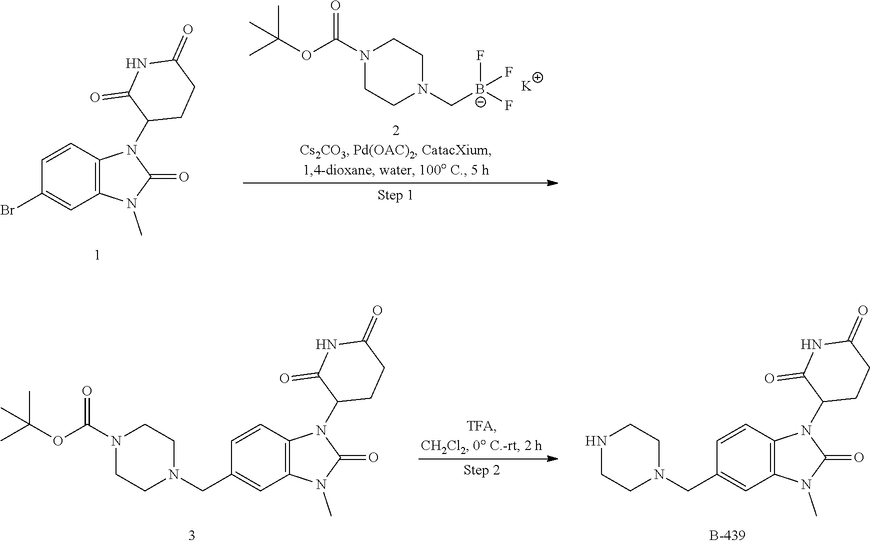 [SMILES: Cn1c(=O)n(C2CCC(=O)NC2=O)c2ccc(Br)cc21 ; CC(C)(C)OC(=O)N1CCN(C[B-](F)(F)F)CC1 ; [K+] ; Cn1c(=O)n(C2CCC(=O)NC2=O)c2ccc(CN3CCN(C(=O)OC(C)(C)C)CC3)cc21 ; Cn1c(=O)n(C2CCC(=O)NC2=O)c2ccc(CN3CCNCC3)cc21]

### Step 1: tert-butyl 4-[[1-(2,6-dioxo-3-piperidyl)-3-methyl-2-oxo-benzimidazol-5-yl]methyl]piperazine-1-carboxylate (3)

Into a 25 ml pressure tube containing a well-stirred solution of potassium ((4-(tert-butoxycarbonyl) piperazin-1-yl)methyl)trifluoroborate (2, 113.18 mg, 369.65 μmol) and 3-(5-bromo-3-methyl-2-oxo-benzimidazol-1-yl)piperidine-2,6-dione (1, 50 mg, 147.86 μmol) in 1,4-dioxane (5 mL) and water (0.5 mL) cesium carbonate (144.53 mg, 443.58 μmol) was added under nitrogen atmosphere. The reaction mixture was degassed by bubbling nitrogen gas for 5 min. Then Palladium (II)acetate (3.32 mg, 14.79 μmol) and CataCXium (1.78 mg, 4.95 μmol) was added. The reaction mixture was stirred at 100° C. After 5 h, the reaction mixture was cooled, filtered through Celite and washed thoroughly with EtOAc. The filtrate was concentrated under reduced pressure and triturated with MTBE (10 mL), filtered, dried and purified by reverse phase prep HPLC (Column: XSelect C18 (250×19) mm, 5 micron; Mobile phase A: 0.1% TFA in water; Mobile phase B: Acetonitrile] to afford tert-butyl 4-[[1-(2,6-dioxo-3-piperidyl)-3-methyl-2-oxo-benzimidazol-5-yl]methyl]piperazine-1-carboxylate (3, 55 mg, 95.83 μmol, 65% yield, TFA salt) as an off-white solid.

LCMS (ES+): m/z 458.2 [M+H]⁺.

### Step 2: 3-[3-methyl-2-oxo-5-(piperazin-1-ylmethyl)benzimidazol-1-yl]piperidine-2,6-dione (B-439)

3-[3-methyl-2-oxo-5-(piperazin-1-ylmethyl)benzimidazol-1-yl]piperidine-2,6-dione (B-439, 610 mg, 1.29 mmol, 99% yield, TFA salt) was synthesized from tert-butyl 4-[[1-(2,6-dioxo-3-piperidyl)-3-methyl-2-oxo-benzimidazol-5-yl]methyl]piperazine-1-carboxylate (3, 630 mg, 1.30 mmol) in a similar fashion to Compound A-62, except using 29 eq. TFA and triturating the product with diethyl ether. LCMS (ES+): m/z 358.2 [M+H]⁺.

### 3-[1-methyl-2-oxo-6-(4-piperidyl)-8,9-dihydro-7H-imidazo[4,5-f]quinolin-3-yl]piperidine-2,6-dione (B-440)

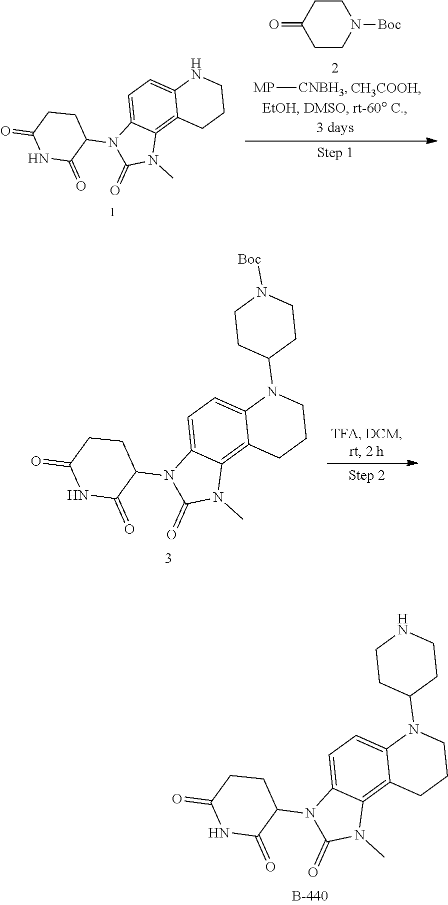

### Step 1: tert-butyl 4-[3-(2,6-dioxo-3-piperidyl)-1-methyl-2-oxo-8,9-dihydro-7H-imidazo[4,5-f]quinolin-6-yl]piperidine-1-carboxylate (3)

Into a 10 mL single neck round bottom flask containing a well-stirred solution of 3-(1-methyl-2-oxo-6,7,8,9-tetrahydroimidazo[4,5-f]quinolin-3-yl)piperidine-2,6-dione (1, 120 mg, 355.03 μmol) and tert-butyl 4-oxopiperidine-1-carboxy late (2, 212.22 mg, 1.07 mmol) in anhydrous DMSO (3 mL) and ethanol (3 mL) was added acetic acid (630.00 mg, 10.49 mmol, 600.00 μL). After 2 h, MP-cyano borohydride (0.35 g, 710.06 μmol, 2 mmol/g) was added. After 24 h, additional quantity of tert-butyl 4-oxopiperidine-1-carboxylate (2, 212.22 mg, 1.07 mmol) was added and heated at 60° C. for 48 h. The reaction mixture was filtered, concentrated and purified by reverse phase column chromatography [Purification method: Silicycle C18 column, Mobile phase A: 0.1% Formic acid in water; Mobile phase B: MeCN] to afford tert-butyl 4-[3-(2,6-dioxo-3-piperidyl)-1-methyl-2-oxo-8,9-dihydro-7H-imidazo[4,5-f]quinolin-6-yl]piperidine-1-carboxylate (3, 60 mg, 111.08 μmol, 31% yield) as an off-white solid.

LCMS (ES+): m/z 498.2 [M+H]⁺.

### Step 2: 3-[1-methyl-2-oxo-6-(4-piperidyl)-8,9-dihydro-7H-imidazo[4,5-f]quinolin-3-yl]piperidine-2,6-dione (B-440)

3-[1-methyl-2-oxo-6-(4-piperidyl)-8,9-dihydro-7H-imidazo[4,5-f]quinolin-3-yl]piperidine-2,6-dione (B-440, 60 mg, 114.75 μmol, 95% yield, TFA salt) was synthesized from tert-butyl 4-[3-(2,6-dioxo-3-piperidyl)-1-methyl-2-oxo-8,9-dihydro-7H-imidazo[4,5-f]quinolin-6-yl]piperidine-1-carboxylate (3, 60 mg, 111.08 μmol) in a similar fashion to Compound A-62, except using 58 eq. TFA. LCMS (ES+): m/z 398.2 [M+H]⁺.

### 3-(5-((S)-azepan-4-yl)-2-oxobenzo[cd]indol-1(2H)-yl)piperidine-2,6-dione (B-441)

Configurations are arbitrarily assigned.

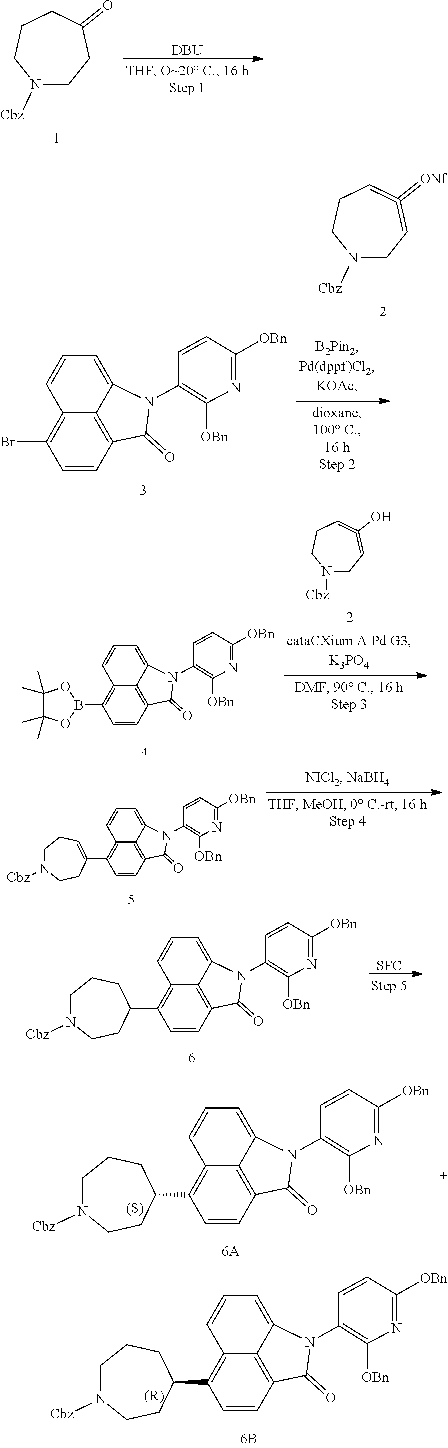

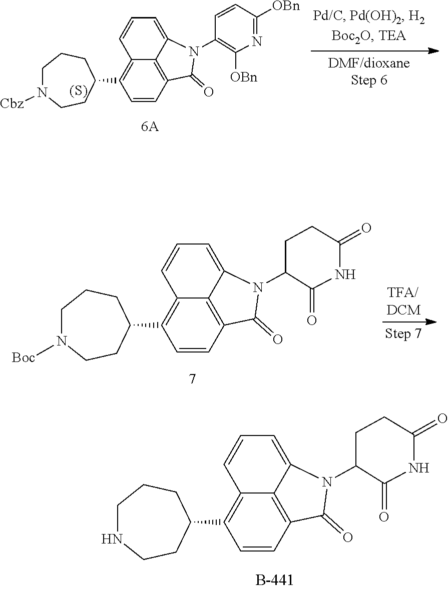

### Step 1: benzyl 4-(((perfluorobutyl)sulfonyl)oxy)-2,3,6,7-tetrahydro-1H-azepine-1-carboxylate (2)

To a mixture of benzyl 4-oxoazepane-1-carboxylate (1, 5 g, 20.22 mmol) and DBU (15.39 g, 101.10 mmol, 15.09 mL) in THF (30 mL) was added 1,1,2,2,3,3,4,4,4-nonafluorobutane-1-sulfonyl fluoride (30.54 g, 101.10 mmol, 17.45 mL) drop-wise at 0° C. under N₂. The mixture was stirred at 20° C. for 16 h. The reaction mixture was filtered and concentrated under reduced pressure. The residue was purified by column chromatography (SiO₂, Petroleum ether:Ethyl acetate=1:0 to 5:1) to afford benzyl 4-(((perfluorobutyl)sulfonyl)oxy)-2,3,6,7-tetrahydro-1H-azepine-1-carboxylate (2, 8 g, 15.14 mmol, 75% yield) as a colorless oil.

### Step 2:1-(2,6-bis(benzyloxy)pyridin-3-yl)-5-(4,4,5,5-tetramethyl-1,3,2-dioxaborolan-2-yl)benzo[cd]indol-2 (1H)-one (4)

To a solution of 1-(2,6-bis(benzyloxy)pyridin-3-yl)-5-bromobenzo[cd]indol-2 (1H)-one (3, 2 g, 3.72 mmol), B₂Pin₂ (1.89 g, 7.44 mmol), Pd(dppf)Cl₂ (272.31 mg, 372.16 μmol), KOAc (1.10 g, 11.16 mmol, 697.92 μL) in dioxane (20 mL). The mixture was stirred at 90° C. for 16 h under N₂. The reaction mixture was filtered and concentrated under reduced pressure. The residue was purified by column chromatography (SiO₂, Petroleum ether:Ethyl acetate=1:0 to 5:1) to afford 1-(2,6-bis(benzyloxy)pyridin-3-yl)-5-(4,4,5,5-tetramethyl-1,3,2-dioxaborolan-2-yl)benzo[cd]indol-2 (1H)-one (4, 1.8 g, 3.08 mmol, 83% yield) as a yellow solid.

LCMS (ESI): m/z 585.2 [M+H]⁺.

### Step 3: benzyl 4-(1-(2,6-bis(benzyloxy)pyridin-3-yl)-2-oxo-1,2-dihydrobenzo[cd]indol-5-yl)-2,3,6,7-tetrahydro-1H-azepine-1-carboxylate (5)

To a solution of benzyl 4-(((perfluorobutyl)sulfonyl)oxy)-2,3,6,7-tetrahydro-1H-azepine-1-carboxylate (2, 1.28 g, 2.42 mmol), 1-(2,6-bis(benzyloxy)pyridin-3-yl)-5-(4,4,5,5-tetramethyl-1,3,2-dioxaborolan-2-yl)benzo[cd]indol-2 (1H)-one (4, 1.7 g, 2.91 mmol) in DMF (10 mL) was added K₃PO₄ (1.54 g, 7.27 mmol) and cataCXium A Pd G₃ (176.77 mg, 242.39 μmol). The mixture was stirred at 90° C. for 16 h under N₂. The residue was diluted with H₂O (150 mL) and extracted with ethyl acetate (150 mL×2). The combined organic layers were washed with brine (150 mL×3), dried over Na₂SO₄, filtered and concentrated under reduced pressure. The residue was purified by column chromatography (SiO₂, Petroleum ether:Ethyl acetate=100:1 to 3:1) to afford benzyl 4-(1-(2,6-bis(benzyloxy)pyridin-3-yl)-2-oxo-1,2-dihydrobenzo[cd]indol-5-yl)-2,3,6,7-tetrahydro-1H-azepine-1-carboxylate (5, 1 g, 1.45 mmol, 60% yield) as a yellow solid. LCMS (ESI): m/z 688.3 [M+H]⁺.

### Step 4 and Step 5: (S)-benzyl 4-(1-(2,6-bis(benzyloxy)pyridin-3-yl)-2-oxo-1,2-dihydrobenzo[cd]indol-5-yl)azepane-1-carboxylate (6A) and (R)-benzyl 4-(1-(2,6-bis(benzyloxy)pyridin-3-yl)-2-oxo-1,2-dihydrobenzo[cd]indol-5-yl))azepane-1-carboxylate (6B)

To a solution of benzyl 4-(1-(2,6-bis(benzyloxy)pyridin-3-yl)-2-oxo-1,2-dihydrobenzo[cd]indol-5-yl)-2,3,6,7-tetrahydro-1H-azepine-1-carboxylate (5, 1 g, 1.45 mmol) in THF (4 mL) and MeOH (20 mL) was added NiCl₂·6H₂O (691.18 mg, 2.91 mmol) at 0° C. After stirring for 20 min, NaBH₄ (825.10 mg, 21.81 mmol, 771.12 μL) was added. The reaction mixture was stirred for 12 h at 20° C. The reaction mixture was quenched by addition of saturated HCl (100 mL) at 0° C., then extracted with ethyl acetate (100 mL×3). The combined organic layers were washed with brine (100 mL×2), dried over Na₂SO₄, filtered and concentrated under reduced pressure. The residue was purified by prep-TLC (SiO2, Petroleum ether:Ethyl acetate=3:1) to afford product. The product was purified by SFC (Sample preparation: Add MeOH 50 ml into sample Instrument: Waters 80Q Mobile Phase: 60% MeOH+ACN (0.1% NH₃·H₂O) in Supercritical CO₂ Flow Rate: 70 g/min Cycle Time: 3.3 min, total time: 60 min Single injection volume: 3.0 ml Back Pressure: 100 bar to keep the CO₂ in Supercritical flow”) to afford(S)-benzyl 4-(1-(2,6-bis(benzyloxy)pyridin-3-yl)-2-oxo-1,2-dihydrobenzo[cd]indol-5-yl)azepane-1-carboxylate (6A, 183 mg, 265.30 μmol, 18% yield) as a yellow oil and (R)-benzyl 4-(1-(2,6-bis(benzyloxy)pyridin-3-yl)-2-oxo-1,2-dihydrobenzo[cd]indol-5-yl)azepane-1-carboxylate (6B, 138 mg, 200.06 μmol, 14% yield) as a yellow oil. LCMS (ESI): m/z 690.3.4 [M+H]⁺.

### Step 6: (4S)-tert-butyl 4-(1-(2,6-dioxopiperidin-3-yl)-2-oxo-1,2-dihydrobenzo[cd]indol-5-yl)azepane-1-carboxylate (7)

To a solution of benzyl(S)-benzyl 4-(1-(2,6-bis(benzyloxy)pyridin-3-yl)-2-oxo-1,2-dihydrobenzo[cd]indol-5-yl)azepane-1-carboxylate (6A, 120 mg, 173.96 μmol) in dioxane (2 mL), DMF (2 mL) was added Pd/C (10%) (100 mg) and Pd(OH)₂/C (10%) (100 mg), Boc₂O (45.56 mg, 208.76 μmol, 47.91 μL), TEA (35.21 mg, 347.93 μmol, 48.49 μL). The mixture was stirred at 20° C. for 16 h under H₂ (15 Psi). The reaction mixture was filtered and concentrated under reduced pressure. The residue was purified by column chromatography (SiO₂, Petroleum ether:Ethyl acetate=1:0 to 1:1) to afford (4S)-tert-butyl 4-(1-(2,6-dioxopiperidin-3-yl)-2-oxo-1,2-dihydrobenzo[cd]indol-5-yl)azepane-1-carboxylate (7, 35 mg, 73.29 μmol, 42% yield) as a white solid. LCMS (ESI): m/z 422.2 [M-^{t}Bu+H]⁺.

### Step 7: 3-(5-((S)-azepan-4-yl)-2-oxobenzo[cd]indol-1(2H)-yl)piperidine-2,6-dione (B-441)

To a solution of (4S)-tert-butyl 4-(1-(2,6-dioxopiperidin-3-yl)-2-oxo-1,2-dihydrobenzo/cd]indol-5-yl)azepane-1-carboxylate (7, 35 mg, 73.29 μmol) in EtOAc (1 mL) was added HCl/EtOAc (4 M, 1 mL) at 0° C. The mixture was stirred at 20° C. for 1 h. The reaction mixture was concentrated under reduced pressure to afford 3-(5-((S)-azepan-4-yl)-2-oxobenzo[cd]indol-1(2H)-yl)piperidine-2,6-dione (B-441, 30 mg, 72.48 μmol, 99% yield), which was used in the next step without further purification. LCMS (ESI): m/z 378.2 [M+H]⁺.

### 3-(5-((R)-azepan-4-yl)-2-oxobenzo[cd]indol-1(2H)-yl)piperidine-2,6-dione (B-442)

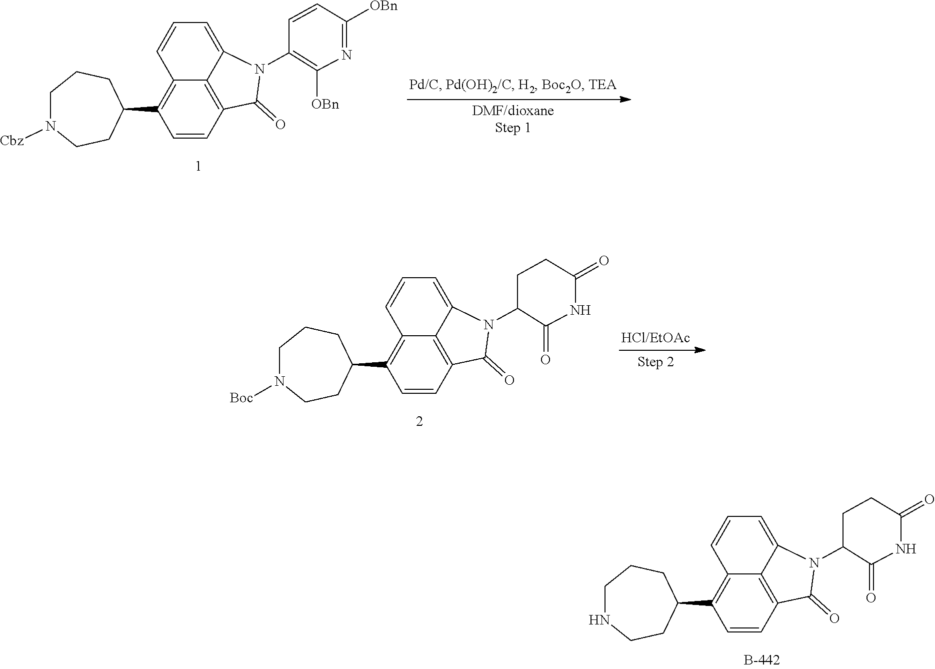 [SMILES: O=C(OCc1ccccc1)N1CCC[C@@H](c2ccc3c4c(cccc24)N(c2ccc(OCc4ccccc4)nc2OCc2ccccc2)C3=O)CC1 ; CC(C)(C)OC(=O)N1CCC[C@@H](c2ccc3c4c(cccc24)N(C2CCC(=O)NC2=O)C3=O)CC1 ; O=C1CCC(N2C(=O)c3ccc([C@@H]4CCCNCC4)c4cccc2c34)C(=O)N1]

### Step 1: (4R)-tert-butyl 4-(1-(2,6-dioxopiperidin-3-yl)-2-oxo-1,2-dihydrobenzo[cd]indol-5-yl)azepane-1-carboxylate (2)

To a solution of (R)-benzyl 4-(1-(2,6-bis(benzyloxy)pyridin-3-yl)-2-oxo-1,2-dihydrobenzo[cd]indol-5-yl)azepane-1-carboxylate (1, 100 mg, 144.97 μmol) in DMF (5 mL) was added Pd/C (10%) (50 mg) and Pd(OH)₂/C (10%) (50 mg), Boc₂O (37.97 mg, 173.96 μmol, 39.92 μL), TEA (29.34 mg, 289.94 μmol, 40.41 μL). The mixture was stirred at 20° C. for 16 h under H₂ (15 Psi). The reaction mixture was filtered and concentrated under reduced pressure. The residue was purified by column chromatography (SiO2, Petroleum ether:Ethyl acetate=1:0 to 1:1) to afford (4R)-tert-butyl 4-(1-(2,6-dioxopiperidin-3-yl)-2-oxo-1,2-dihydrobenzo[cd]indol-5-yl)azepane-1-carboxylate (2, 50 mg, 104.70 μmol, 72% yield) as a white solid.

LCMS (ESI): m/z 422.2 [M−^{t}Bu+H]⁺.

### Step 2: 3-(5-((R)-azepan-4-yl)-2-oxobenzo[cd]indol-1(2H)-yl)piperidine-2,6-dione (B-442)

To a solution of (4R)-tert-butyl 4-(1-(2,6-dioxopiperidin-3-yl)-2-oxo-1,2-dihydrobenzo[cd]indol-5-yl)azepane-1-carboxylate (2, 60 mg, 125.64 μmol) in EtOAc (1 mL) was added HCl/EtOAc (4 M, 1 mL) at 0° C. The mixture was stirred at 20° C. for 1 h. The reaction mixture was concentrated under reduced pressure to afford 3-(5-((R)-azepan-4-yl)-2-oxobenzo[cd]indol-1(2H)-yl)piperidine-2,6-dione (B-442, 45 mg, 108.72 μmol, 87% yield, HCl salt), which was used to the next step without further purification. LCMS (ESI): m/z 378.2 [M+H]⁺.

### 3-[5-[4-(2-aminoethyl)-1-piperidyl]-3-methyl-indol-1-yl]piperidine-2,6-dione (B-443)

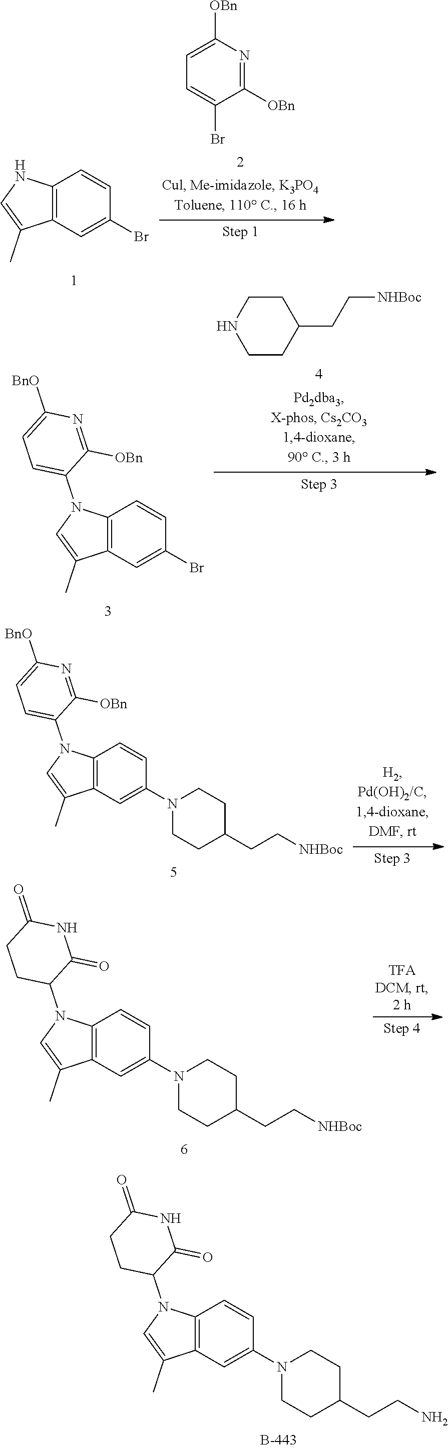

### Step 1: 5-bromo-1-(2,6-dibenzyloxy-3-pyridyl)-3-methyl-indole (3)

Into a 250 mL pressure tube containing a well-stirred solution of 5-bromo-3-methyl-1H-indole (1, 1.5 g, 7.14 mmol) and 2,6-dibenzyloxy-3-bromo-pyridine (2, 2.64 g, 7.14 mmol) in anhydrous toluene (15 mL) was added potassium phosphate (3.03 g, 14.28 mmol) at rt under nitrogen atmosphere. Then, copper (I) iodide (271.98 mg, 1.43 mmol) and 1-methyl imidazole (117.25 mg, 1.43 mmol) were added. The mixture was stirred at 130° C. After 16 h, the reaction mixture was filtered through Celite and washed with ethyl acetate (150 mL). The filtrate was concentrated and purified by flash silica gel column chromatography (25% DCM in pet ether) to afford 5-bromo-1-(2,6-dibenzyloxy-3-pyridyl)-3-methyl-indole (3, 1.9 g, 3.49 mmol, 49% yield) as a gummy solid.

LCMS (ES+): m/z 501.5 [M+H]⁺.

### Step 2: tert-butyl N-[2-[1-[1-(2,6-dibenzyloxy-3-pyridyl)-3-methyl-indol-5-yl]-4-piperidyl]ethyl]carbamate (5)

tert-butyl N-[2-[1-[1-(2,6-dibenzyloxy-3-pyridyl)-3-methyl-indol-5-yl]-4-piperidyl]ethyl]carbamate (5, 850 mg, 1.16 mmol, 52% yield) was synthesized from 5-bromo-1-(2,6-dibenzyloxy-3-pyridyl)-3-methyl-indole (3, 1.2 g, 2.20 mmol) and tert-butyl N-[2-(4-piperidyl)ethyl]carbamate (4, 503.33 mg, 2.20 mmol) in a similar fashion to Compound B-180, except using 0.2 eq. tris(dibenzylideneacetone) dipalladium (0) and 0.2 eq. XPhos.

LCMS (ES+): m/z 648.0 [M+H]⁺.

### Step 3: tert-butyl N-[2-[1-[1-(2,6-dioxo-3-piperidyl)-3-methyl-indol-5-yl]-4-piperidyl]ethyl]carbamate (6)

tert-butyl N-[2-[1-[1-(2,6-dioxo-3-piperidyl)-3-methyl-indol-5-yl]-4-piperidyl]ethyl]carbamate (6, 180 mg, 327.43 μmol, 57% yield, Formic acid salt) was synthesized from tert-butyl N-[2-[1-[1-(2,6-dibenzyloxy-3-pyridyl)-3-methyl-indol-5-yl]-4-piperidyl]ethyl]carbamate (5, 460 mg, 576.05 μmol) in a similar fashion to Compound B-103, except using 20 wt. % palladium hydroxide on carbon (404.49 mg, 576.05 μmol). The material was purified by reverse phase column chromatography (Purification method: Silicycle C18 column; Mobile phase A: 0.1% formic acid in water; Mobile phase B: MeCN). LCMS (ES+): m/z 469.2 [M+H]⁺.

### Step 4: 3-[5-[4-(2-aminoethyl)-1-piperidyl]-3-methyl-indol-1-yl]piperidine-2,6-dione (B-443)

3-[5-[4-(2-aminoethyl)-1-piperidyl]-3-methyl-indol-1-yl]piperidine-2,6-dione (B-443, 550 mg, 1.07 mmol, 98.49% yield, TFA salt) was synthesized from tert-butyl N-[2-[1-[1-(2,6-dioxo-3-piperidyl)-3-methyl-indol-5-yl]-4-piperidyl]ethyl]carbamate (6, 700 mg, 1.09 mmol) in a similar fashion to Compound A-62, except using 10 eq. TFA. The material was purified by reverse phase prep-HPLC (Purification method: XSelect C18 (150×19.1) mm, 5 microns; Mobile phase A: 0.1% TFA in water; Mobile phase B: MeCN). LCMS (ES+): m/z 369.2 [M+H]⁺.

### 3-[3-methyl-5-(4-piperidyl) indazol-1-yl]piperidine-2,6-dione (B-444)

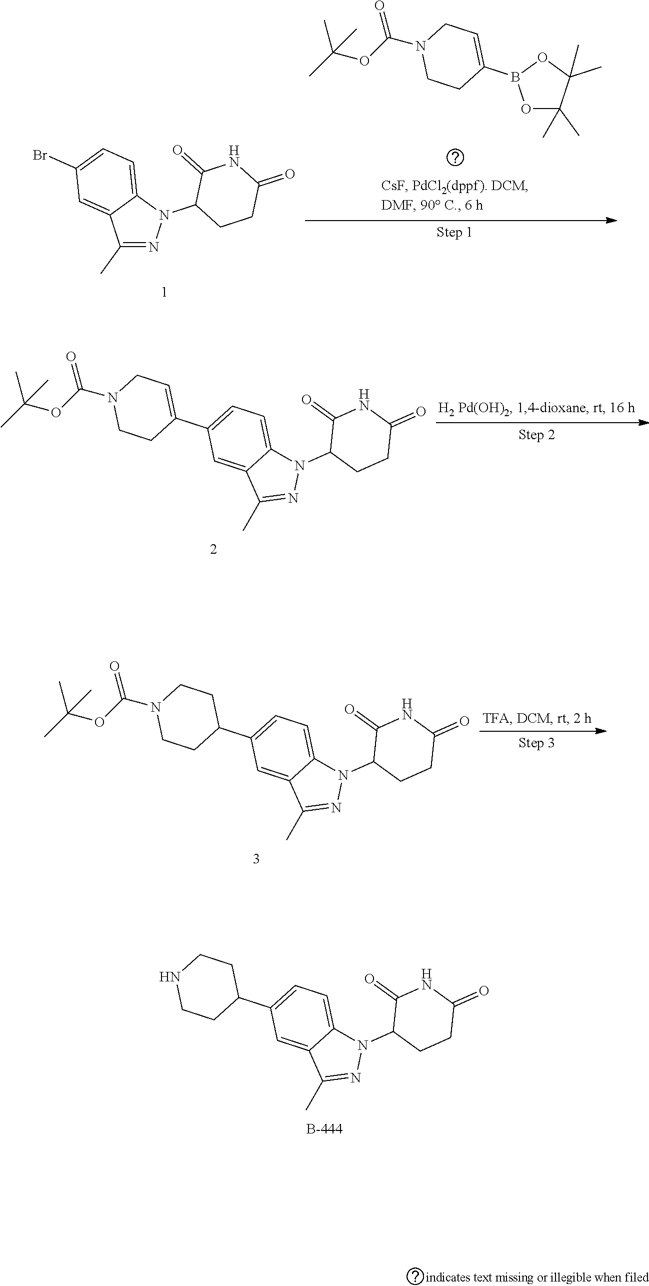 [SMILES: CC(C)(C)OC(=O)N1CC=C(B2OC(C)(C)C(C)(C)O2)CC1 ; Cc1nn(C2CCC(=O)NC2=O)c2ccc(Br)cc12 ; Cc1nn(C2CCC(=O)NC2=O)c2ccc(C3=CCN(C(=O)OC(C)(C)C)CC3)cc12 ; Cc1nn(C2CCC(=O)NC2=O)c2ccc(C3CCN(C(=O)OC(C)(C)C)CC3)cc12 ; Cc1nn(C2CCC(=O)NC2=O)c2ccc(C3CCNCC3)cc12]

### Step 1: tert-butyl 4-[1-(2,6-dioxo-3-piperidyl)-3-methyl-indazol-5-yl]-3,6-dihydro-2H-pyridine-1-carboxylate (2)

tert-butyl 4-[1-(2,6-dioxo-3-piperidyl)-3-methyl-indazol-5-yl]-3,6-dihydro-2H-pyridine-1-carboxylate (2, 372 mg, 777.05 μmol, 63% yield) was synthesized from 3-(5-bromo-3-methyl-indazol-1-yl)piperidine-2,6-dione (1, 400 mg, 1.24 mmol) and tert-butyl 4-(4,4,5,5-tetramethyl-1,3,2-dioxaborolan-2-yl)-3,6-dihydro-2H-pyridine-1-carboxylate (1a, 579.72 mg, 1.87 mmol) in a similar fashion to Compound B-133, except using 2.5 eq. CsF, and 0.3 eq. 1,1′-Bis(diphenylphosphino)ferrocene]dichloropalladium(II) complex with dichloromethane.

LCMS (ES+): m/z 425.6 [M+H]⁺.

### Step 2: tert-butyl 4-[1-(2,6-dioxo-3-piperidyl)-3-methyl-indazol-5-yl]piperidine-1-carboxylate (3)

tert-butyl4-[1-(2,6-dioxo-3-piperidyl)-3-methyl-indazol-5-yl]piperidine-1-carboxylate (3, 350 mg, 533.40 μmol, 57% yield, 65% purity) was synthesized from tert-butyl 4-[1-(2,6-dioxo-3-piperidyl)-3-methyl-indazol-5-yl]-3,6-dihydro-2H-pyridine-1-carboxylate (2, 450 mg, 939.98 μmol) in a similar fashion to Compound B-103, except using 20 wt. % palladium hydroxide on carbon (462.02 mg, 657.99 μmol). UP-LCMS (ES+): m/z 427.7 [M+H]⁻.

### Step 3: 3-[3-methyl-5-(4-piperidyl) indazol-1-yl]piperidine-2,6-dione (B-444)

3-[3-methyl-5-(4-piperidyl) indazol-1-yl]piperidine-2,6-dione (B-444, 230 mg, 510.22 μmol, 96% yield, TFA Salt) was synthesized from tert-butyl 4-[1-(2,6-dioxo-3-piperidyl)-3-methyl-indazol-5-yl]piperidine-1-carboxylate (3, 350 mg, 533.40 μmol) in a similar fashion to Compound A-62, except using 20 eq. TFA. The material was purified by reverse phase prep-HPLC (Purification method: Column: XSelect C18 (150×19) mm, 5 micron; Mobile phase A: 0.1% TFA in water; Mobile phase B: MeCN). LCMS (ES+): m/z 327.7 [M+H]⁺.

### 3-[3-methyl-2-oxo-5-(4-piperidylmethyl)benzimidazol-1-yl]piperidine-2,6-dione (B-445)

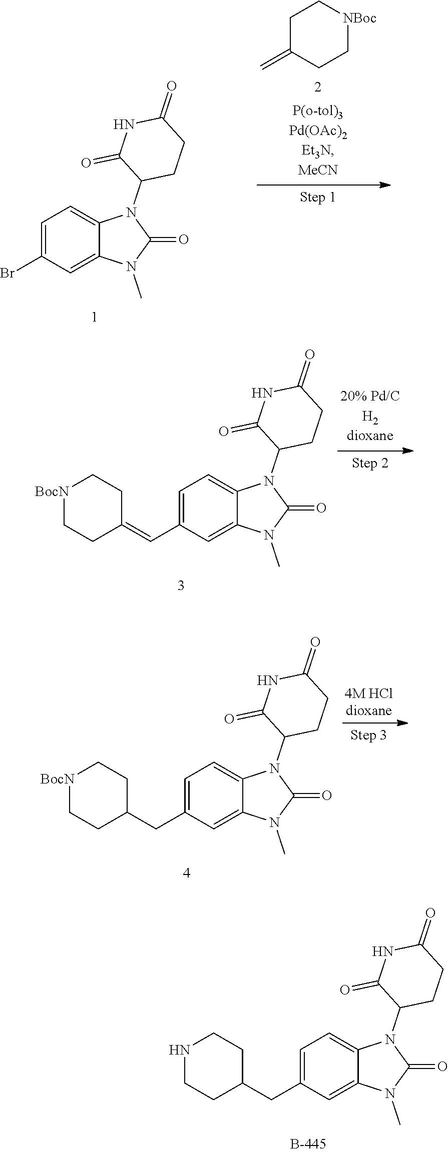 [SMILES: C=C1CCN(C(=O)OC(C)(C)C)CC1 ; Cn1c(=O)n(C2CCC(=O)NC2=O)c2ccc(Br)cc21 ; Cn1c(=O)n(C2CCC(=O)NC2=O)c2ccc(C=C3CCN(C(=O)OC(C)(C)C)CC3)cc21 ; Cn1c(=O)n(C2CCC(=O)NC2=O)c2ccc(CC3CCN(C(=O)OC(C)(C)C)CC3)cc21 ; Cn1c(=O)n(C2CCC(=O)NC2=O)c2ccc(CC3CCNCC3)cc21]

### Step 1: 4-[[1-(2,6-dioxo-3-piperidyl)-3-methyl-2-oxo-benzimidazol-5-yl]methylene]piperidine-1-carboxylate (3)

In a 25 mL seal-tubed, triethylamine (2.90 g, 28.70 mmol, 4 mL) was added to a stirring solution of 3-(5-bromo-3-methyl-2-oxo-benzimidazol-1-yl)piperidine-2,6-dione (1, 1 g, 2.96 mmol) and tert-butyl 4-methylenepiperidine-1-carboxylate (2, 700.06 mg, 3.55 mmol) in anhydrous acetonitrile (10 mL). The mixture was degassed with nitrogen for 5 min before tris(o-tolyl) phosphine (90.01 mg, 295.72 μmol) and palladium(II)acetate (99.59 mg, 443.58 μmol) were added. The reaction mixture was further degassed for 5 min and then heated at 90° C. for 16 h. The reaction was filtered through Celite, which was washed with ethyl acetate (100 mL). The filtrate was concentrated and purified by reversed-phase column chromatography (C18, 120 g, 0.1% TFA in water/acetonitrile) to afford tert-butyl 4-[[1-(2,6-dioxo-3-piperidyl)-3-methyl-2-oxo-benzimidazol-5-yl]methylene]piperidine-1-carboxy late (3, 600 mg, 1.14 mmol, 38% yield) as a yellow colored solid. LCMS (ES⁺): m/z 355.2 [M−Boc+H]⁺.

### Step 2: tert-butyl 4-[[1-(2,6-dioxo-3-piperidyl)-3-methyl-2-oxo-benzimidazol-5-yl]methyl]piperidine-1-carboxylate (4)

tert-butyl 4-[1-(2,6-dioxo-3-piperidyl)-3-methyl-2-oxo-benzimidazol-5-yl]methyl]piperidine-1-carboxylate (4, 540 mg, 1.08 mmol, 98% yield) was synthesized from tert-butyl 4-[[1-(2,6-dioxo-3-piperidyl)-3-methyl-2-oxo-benzimidazol-5-yl]methylene]piperidine-1-carboxylate (3, 600 mg, 1.10 mmol) in a similar fashion to Compound B-103, except using 20 wt. % palladium hydroxide on carbon (0.79 eq.). The material was used in the next step without purification. LCMS (ES+): m/z 357.2 [M−Boc+H]⁺.

### Step 3: 3-[3-methyl-2-oxo-5-(4-piperidylmethyl)benzimidazol-1-yl]piperidine-2,6-dione (B-445)

3-[3-methyl-2-oxo-5-(4-piperidylmethyl)benzimidazol-1-yl]piperidine-2,6-dione (B-445, 500 mg, 1.09 mmol, 98% yield, HCl salt) was synthesized from tert-butyl 4-[[1-(2,6-dioxo-3-piperidyl)-3-methyl-2-oxo-benzimidazol-5-yl]methyl]piperidine-1-carboxylate (4, 560 mg, 1.12 mmol) in a similar fashion to (6, step 3 of Compound A-148/Compound 149, using hydrogen chloride solution (4 M in dioxane, 2 mL) and stirring at rt for 3 h. The residue was washed with diethyl ether (2×10 mL) and concentrated completely under reduced pressure to afford the product as a pale-yellow solid. LCMS (ES+): m/z 357.2 [M+H]⁺.

### 2-[1-[1-(2,6-dioxo-3-piperidyl)-3-methyl-2-oxo-benzimidazol-5-yl]-4-piperidyl]acetaldehyde (B-446)

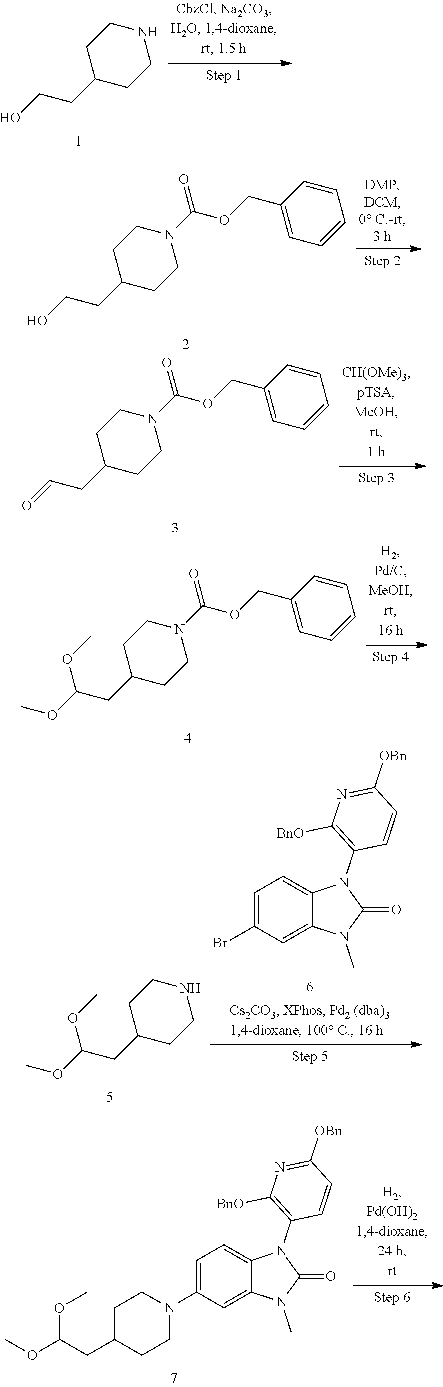

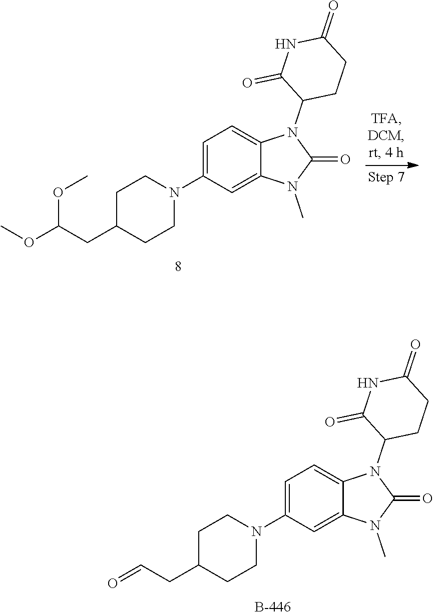 [SMILES: COC(CC1CCN(c2ccc3c(c2)n(C)c(=O)n3C2CCC(=O)NC2=O)CC1)OC ; Cn1c(=O)n(C2CCC(=O)NC2=O)c2ccc(N3CCC(CC=O)CC3)cc21]

### Step 1: benzyl 4-(2-hydroxyethyl)piperidine-1-carboxylate (2)

Into a 250 mL single neck round bottom flask containing a well-stirred solution of 2-(4-piperidyl)ethanol (1, 3 g, 23.22 mmol) in anhydrous 1,4-dioxane (45 mL) was added sodium carbonate (4.50 g, 42.46 mmol) in water (45 mL) and the solution was cooled to 0° C. Then benzyl chloroformate (4.36 g, 25.54 mmol) was added and stirring continued at rt for 1.5 h. The solvents were removed from the reaction mixture and the residue was diluted with EtOAc (100 mL). The organic layer was washed with saturated NaHCO₃ solution (2×75 mL) followed by 1.5N HCl solution (2×75 mL), brine solution (2×75 mL), dried with anhydrous Na₂SO₄ and filtered. The filtrate was concentrated and purified by flash silica gel (60-120 mesh) column chromatography (85% EtOAc in pet ether) to afford benzyl 4-(2-hydroxyethyl)piperidine-1-carboxylate (2, 5.8 g, 21.97 mmol, 94.60% yield) as colorless thick liquid.

LCMS (ES+): m/z 264.2 [M+H]⁺.

### Step 2: benzyl 4-(2-oxoethyl)piperidine-1-carboxylate (3)

Into a 500 mL single neck round bottom flask containing a well-stirred solution of benzyl 4-(2-hydroxyethyl)piperidine-1-carboxylate (2, 5.8 g, 21.67 mmol) in anhydrous DCM (90 mL) was added Dess-Martin Periodinane (10.11 g, 23.84 mmol) at 0° C. The reaction mixture was stirred at rt. After 3 h, the reaction mixture was quenched with saturated NaHCO₃ solution (40 mL) and filtered through a Celite. The filtrate was diluted with DCM (150 mL) and washed with brine solution (2×50 mL), dried over anhydrous Na₂SO₄, filtered, concentrated and purified by flash silica gel (60-120 mesh) column chromatography (62% EtOAc in pet ether) to afford benzyl 4-(2-oxoethyl)piperidine-1-carboxylate (3, 4.3 g, 14.81 mmol, 68% yield) as color less thick liquid.

¹HNMR (400 MHz, CDCl₃): δ 9.79 (t, J=1.60 Hz, 1H), 7.39-7.32 (m, 5H), 5.14 (s, 2H), 4.19-4.15 (m, 2H), 2.88-2.82 (m, 2H), 2.42-2.40 (m, 2H), 2.13-2.06 (m, 1H), 1.75-1.72 (m, 2H), 1.28-1.21 (m, 2H).

### Step 3: benzyl 4-(2,2-dimethoxyethyl)piperidine-1-carboxylate (4)

Into a 500 mL single neck round bottom flask containing a well-stirred solution of benzyl 4-(2-oxoethyl)piperidine-1-carboxylate (3, 4.3 g, 14.81 mmol) in anhydrous methanol (60 mL) were added trimethoxymethane (7.86 g, 74.05 mmol, 8.12 mL) and p-toluenesulfonic acid monohydrate (281.71 mg, 1.48 mmol, 227.18 μL). The mixture was stirred for 16 h. The reaction was quenched with water (50 mL) at 15° C. and the aqueous layer was extracted with DCM (3×30 mL). The combined organic layer was washed with brine solution (2×25 mL), dried over anhydrous Na₂SO₄, filtered, concentrated and purified by flash silica gel (60-120 mesh) column chromatography (50% EtOAc in pet ether) to afford benzyl 4-(2,2-dimethoxyethyl)piperidine-1-carboxylate (4, 3.9 g, 10.78 mmol, 73% yield) as colorless thick liquid. ¹HNMR (400 MHz, CDCl₃): δ 7.40-7.31 (m, 5H), 5.15 (s, 2H), 4.50-4.48 (m, 1H), 4.29-4.06 (m, 2H), 3.38 (s, 6H), 2.83-2.77 (m, 2H), 1.74-1.67 (m, 2H), 1.64-1.55 (m, 3H), 1.28-1.11 (m, 2H).

### Step 4:4-(2,2-dimethoxyethyl)piperidine (5)

Into a 250 mL single neck round bottom flask containing a well-stirred solution of benzyl 4-(2,2-dimethoxyethyl)piperidine-1-carboxylate (4, 3.9 g, 10.78 mmol) in anhydrous methanol (40 mL) was added 10% palladium on carbon (2.30 g, 2.16 mmol). The suspension was stirred at rt under hydrogen atmosphere (bladder) for 16 h. The reaction mixture was filtered through Celite, washed with methanol (200 mL) and the filtrate was concentrated under reduced pressure to afford 4-(2,2-dimethoxyethyl)piperidine (5, 1.8 g, 10.32 mmol, 96% yield) as colorless syrupy liquid.

LCMS (ES+): m/z 174.4 [M+H]⁺.

### Step 5:1-(2,6-dibenzyloxy-3-pyridyl)-5-[4-(2,2-dimethoxyethyl)-1-piperidyl]-3-methyl-benzimidazol-2-one (7)

1-(2,6-dibenzyloxy-3-pyridyl)-5-[4-(2,2-dimethoxyethyl)-1-piperidyl]-3-methyl-benzimidazol-2-one (7, 0.6 g, 947.72 μmol, 50% yield) was synthesized from 5-bromo-1-(2,6-dibenzyloxy-3-pyridyl)-3-methyl-benzimidazol-2-one (6, 1 g, 1.89 mmol) and 4-(2,2-dimethoxyethyl)piperidine (5, 658.05 mg, 3.77 mmol) in a similar fashion to Compound B-180, except using 0.2 eq. tris(dibenzylideneacetone) dipalladium (0), 0.2 eq. XPhos and heating at 100° C.

LCMS (ES+): m/z 609.9 [M+H]⁺.

### Step 6: 3-[5-[4-(2,2-dimethoxyethyl)-1-piperidyl]-3-methyl-2-oxo-benzimidazol-1-yl]piperidine-2,6-dione (8)

3-[5-[4-(2,2-dimethoxyethyl)-1-piperidyl]-3-methyl-2-oxo-benzimidazol-1-yl]piperidine-2,6-dione (8, 380 mg, 852.96 μmol, 90% yield) was synthesized from 1-(2,6-dibenzyloxy-3-pyridyl)-5-[4-(2,2-dimethoxyethyl)-1-piperidyl]-3-methyl-benzimidazol-2-one (7, 0.6 g, 946.24 μmol) in a similar fashion to Compound B-103, except using 20 wt. % palladium hydroxide on carbon (664.43 mg, 946.24 μmol). LCMS (ES+): m/z 431.7 [M+H]⁺.

### Step 7:2-[1-[1-(2,6-dioxo-3-piperidyl)-3-methyl-2-oxo-benzimidazol-5-yl]-4-piperidyl]acetaldehyde (B-446)

Into a 50 mL single neck round bottom flask containing a well-stirred solution of 3-[5-[4-(2,2-dimethoxyethyl)-1-piperidyl]-3-methyl-2-oxo-benzimidazol-1-yl]piperidine-2,6-dione (8, 380 mg, 852.96 μmol) in anhydrous DCM (2.5 mL) was added TFA (2.22 g, 19.47 mmol, 1.5 mL). After 4 h, the volatiles were removed under reduced pressure, the residue was washed with MTBE (50 mL) and dried to afford 2-[1-[1-(2,6-dioxo-3-piperidyl)-3-methyl-2-oxo-benzimidazol-5-yl]-4-piperidyl]acetaldehyde (B-446, 0.38 g, 572.00 μmol, 67% yield, 75% purity, TFA salt) as off white solid.

LCMS (ES+): m/z 385.6 [M+H]⁺.

### 3-[2-oxo-5-(4-piperidyl)benzo[cd]indol-1-yl]piperidine-2,6-dione (B-447)

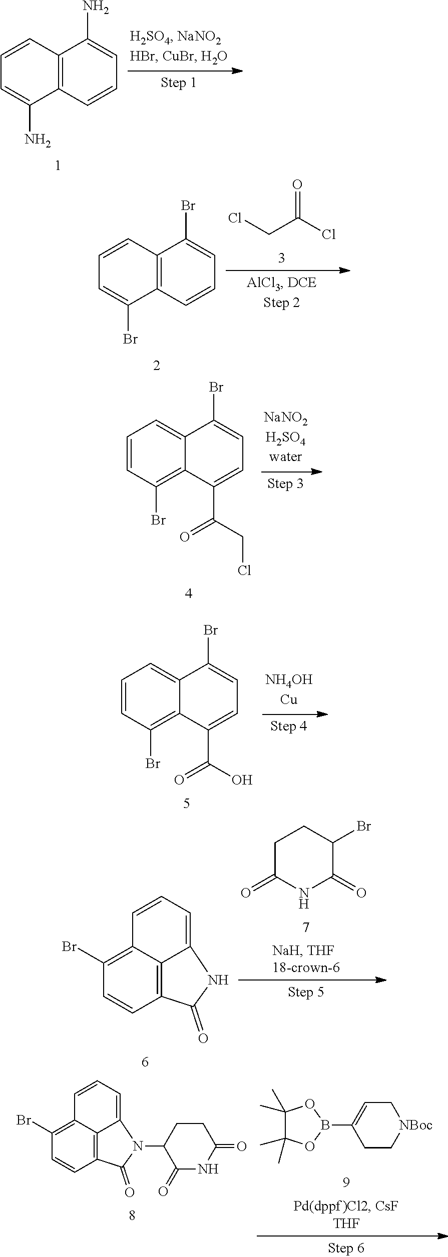

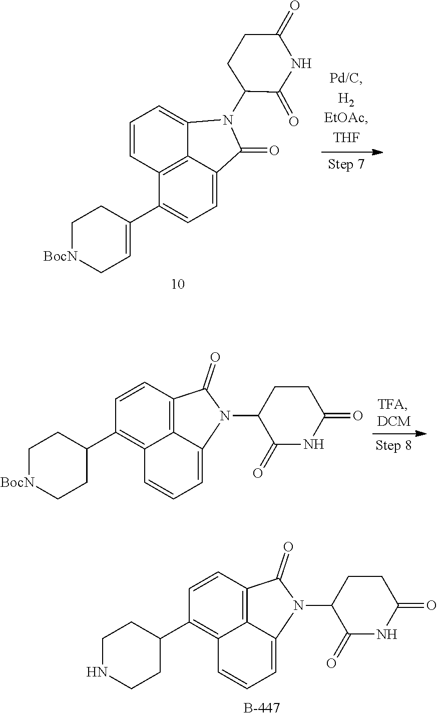 [SMILES: CC(C)(C)OC(=O)N1CC=C(c2ccc3c4c(cccc24)N(C2CCC(=O)NC2=O)C3=O)CC1 ; CC(C)(C)OC(=O)N1CCC(c2ccc3c4c(cccc24)N(C2CCC(=O)NC2=O)C3=O)CC1 ; O=C1CCC(N2C(=O)c3ccc(C4CCNCC4)c4cccc2c34)C(=O)N1]

### Step 1: 1,5-dibromonaphthalene (2)

To a stirred solution of sulfuric acid (93.00 g, 948.17 mmol, 247 mL) and Water (3 L), was added naphthalene-1,5-diamine (1, 150 g, 948.17 mmol) in a portion wise manner at 0° C. for 10 min. To this mixture, a solution of Sodium nitrite (163.56 g, 2.37 mol, 75.37 mL) in Water (100 mL) was added dropwise over 30 min at 0° C. The brown mixture was stirred at 0° C. for 90 min, and then was carefully added over 30 min into another reaction flask containing a solution of Hydrobromic acid (48%) (4.47 kg, 55.25 mol, 3 L) and Copper I Bromide (375.40 g, 2.62 mol, 79.70 mL) at 0° C. in a portion wise manner. The reaction mixture immediately began to evolve gas; it was stirred at 0° C. for 30 min before warming rt, and was heated to 70° C. (internal temperature, using oil bath) until gas evolution ceased (about 1 h), and then stirred at rt. The reaction was filtered and washed with water (5 lit). It was dried under house vacuum to afford brown solid. It was suspended in 20% EtOAc in pet ether (20 lit) and then passed through silica gel column using 60-120 silica gel. The solvent was concentrated under vacuum to afford 1,5-dibromonaphthalene (2, 130 g, 436.42 mmol, 46% yield) as a brown color solid. The material was used in the next step without further characterization.

### Step 2: 2-chloro-1-(4,8-dibromo-1-naphthyl) ethanone (4)

A stirred solution of 1,5-dibromonaphthalene (2, 200 g, 699.39 mmol) in DCE (2400 mL) was cooled to 0° C. and Chloroacetyl chloride (3, 102.69 g, 909.21 mmol, 72.31 mL) was added to it drop wise and the reaction mass was stirred at the same temperature for about 15 min. Aluminium chloride (121.23 g, 909.21 mmol, 49.69 mL) was then added portion wise. Reaction mixture was slowly warmed to rt and stirred for 5 h. After completion, the reaction mixture was quenched with cold water (1 L). DCM (2 L) was added filtered over Celite and the combined filtrate was washed with water, brine, and the DCM layer dried over anhydrous sodium sulfate, filtered and concentrated under reduced pressure. This material stirred in 2% ethyl acetate in pet ether (2 L) for 30 min and the solid filtered and washed with pet ether (2 L) to afford 2-chloro-1-(4,8-dibromo-1-naphthyl) ethanone (4, 200 g, 496.63 mmol, 71% yield) as a brown color solid. LCMS (ES⁺): m/z 360.98 [M+H]⁺.

### Step 3:4,8-dibromonaphthalene-1-carboxylic acid (5)

To a stirred solution of 2-chloro-1-(4,8-dibromo-1-naphthyl)) ethanone (4, 200 g, 551.81 mmol) in H₂SO₄ (2400 mL) was added a solution of Sodium Nitrite (39.98 g, 579.40 mmol, 18.42 mL) in Water (40 mL) dropwise at 0° C. and the reaction mixture was stirred at 25° C. for 2 h. The reaction mixture was then poured into cold water (870 mL) and filtered. The solid was added to ethyl acetate and water solution (1:1, 870:870 mL), the mixture was filtered, over Celite and washed with ethyl acetate (500 mL). The aqueous part was extracted with ethyl acetate (100 mL×2). The combined organic part was washed with brine, dried over sodium sulfate and concentrated under reduced pressure and washed with 10% ethyl acetate in pet ether and dried the compound to afford 4,8-dibromonaphthalene-1-carboxylic acid (5, 160 g, 402.46 mmol, 73% yield) as brown color solid.

LCMS (ES⁻): m/z 327.05 [M−H]⁻.

### Step 4: 5-bromo-1H-benzo[cd]indol-2-one (6)

To a stirred suspension of 4,8-dibromonaphthalene-1-carboxylic acid (5, 160 g, 484.89 mmol) in Ammonium hydroxide (28% solution) (1.98 kg, 56.49 mol, 2.20 L), Copper (8.01 g, 126.07 mmol) was added and the reaction mixture was stirred at 80° C. for 2 h. After completion, the reaction mixture was cooled to rt and acidified with conc. hydrochloric acid (pH 2-3). The resulting suspension was filtered and dried. The material was stirred in 10% ethyl acetate in pet ether for 30 min, then filtered and washed this solid with pet ether to afford 5-bromo-1H-benzo[cd]indol-2-one (6, 110 g, 336.99 mmol, 70% yield, 76% purity) as a brown color solid.

LCMS (ES⁺): m/z 247.84 [M+H]⁺.

### Step 5: 3-(5-bromo-2-oxo-benzo[cd]indol-1-yl)piperidine-2,6-dione (8)

To a stirred solution of 5-bromo-1H-benzo[cd]indol-2-one (6, 10 g, 40.31 mmol) in THF (200 mL) was slowly added NaH (20 g, 869.95 mmol) at 0° C. and stirred for 30 min at 0° C. The solution of 3-bromopiperidine-2,6-dione (7, 40 g, 208.32 mmol) in THF (200 mL) was added to the reaction mixture and stirred for 16 h at 60° C. After completion of reaction, quenched with aq ammonium chloride and extracted with ethyl acetate. The solvent was concentrated under reduced pressure to afford 3-(5-bromo-2-oxo-benzo[cd]indol-1-yl)piperidine-2,6-dione (8, 4.7 g, 12.27 mmol, 30% yield) as light green solid. LCMS (ES⁻): m/z 357.10 [M−H]⁻.

### Step 6: tert-butyl 4-[1-(2,6-dioxo-3-piperidyl)-2-oxo-benzo[cd]indol-5-yl]-3,6-dihydro-2H-pyridine-1-carboxylate (10)

A stirred solution of Cesium fluoride (2.54 g, 16.71 mmol, 615.91 μL) in THF (60 mL) was purged with argon for 10 min. 3-(5-bromo-2-oxo-benzo[cd]indol-1-yl)piperidine-2,6-dione (8, 3.0 g, 8.35 mmol) and tert-butyl 4-(4,4,5,5-tetramethyl-1,3,2-dioxaborolan-2-yl)-3,6-dihydro-2H-pyridine-1-carboxylate (9, 5.68 g, 18.38 mmol) were added to the reaction mixture and again purged with argon for 20 min. cyclopentyl(diphenyl)phosphane; dichloropalladium; iron (611.16 mg, 835.25 μmol) was added to the reaction mixture and stirred for at. After completion of reaction, the reaction mixture filtered through Celite and washed with ethyl acetate. The filtrate was concentrated and purified by column chromotography (davisil silica and 70% ethyl acetate in pet ether) to afford tert-butyl 4-[1-(2,6-dioxo-3-piperidyl)-2-oxo-benzo[cd]indol-5-yl]-3,6-dihydro-2H-pyridine-1-carboxylate (10, 2.5 g, 5.11 mmol, 61% yield) as yellow solid.

LCMS (ES⁻): m/z 460.22 [M−H]⁻.

### Step 7: tert-butyl 4-[1-(2,6-dioxo-3-piperidyl)-2-oxo-benzo[cd]indol-5-yl]piperidine-1-carboxylate (11)

To a stirred solution of tert-butyl 4-[1-(2,6-dioxo-3-piperidyl)-2-oxo-benzo[cd]indol-5-yl]-3,6-dihydro-2H-pyridine-1-carboxylate (10, 250 mg, 541.70 μmol) in EtOAc (5 mL) and THF (5 mL) was degassed with nitrogen gas for 10 min and then Pd/C (288.24 mg, 2.71 mmol) was added at rt. The reaction mixture was stirred under hydrogen atmosphere (balloon pressure) at rt for 16 h. After completion of reaction, the reaction mixture was filtered through Celite and washed with THF (50 mL). The filtrate was concentrated under reduced pressure. The residue was purified by column chromatography using devisil silica, 5% MeOH in DCM as eluent to afford tert-butyl 4-[1-(2,6-dioxo-3-piperidyl)-2-oxo-benzo[cd]indol-5-yl]piperidine-1-carboxylate (11, 210 mg, 392.70 μmol, 72% yield) as colorless gum. LCMS (ES⁺): m/z 462.43 [M−H]⁻.

### Step 8: 3-[2-oxo-S-(4-piperidyl)benzo[cd]indol-1-yl]piperidine-2,6-dione (B-447)

3-[2-oxo-5-(4-piperidyl)benzo[cd]indol-1-yl]piperidine-2,6-dione (B-447, 0.2 g, 443.30 μmol, 98% yield) was synthesized from tert-butyl 4-[1-(2,6-dioxo-3-piperidyl)-2-oxo-benzo[cd]indol-5-yl]piperidine-1-carboxylate (210 mg, 453.05 μmol) in a similar fashion to Compound A-62, except using 10 eq. TFA. The product was triturated using diethyl ether (2×15 mL).

LCMS (ES⁺): m/z 364.85 [M+H]⁺.

### 3-[5-(3-aminopropyl)-2-oxo-benzo[cd]indol-1-yl]piperidine-2,6-dione (B-448)

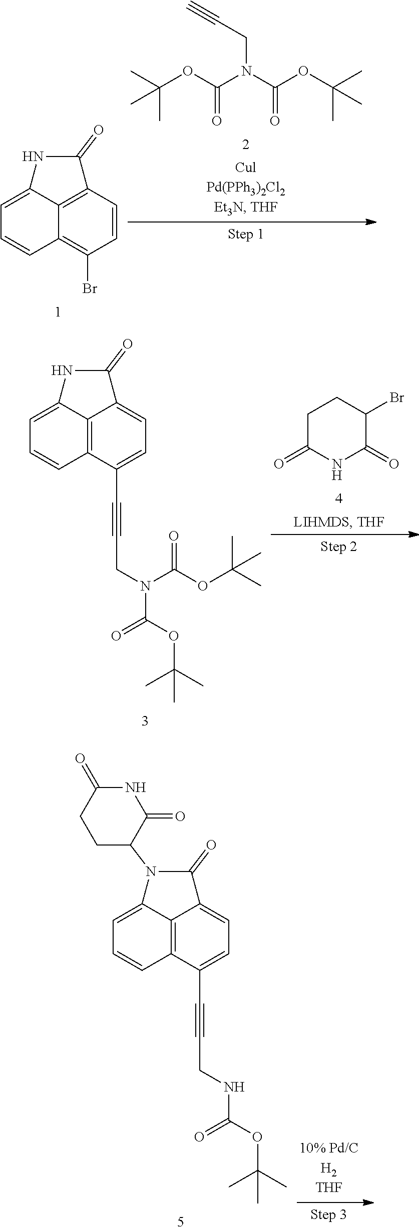

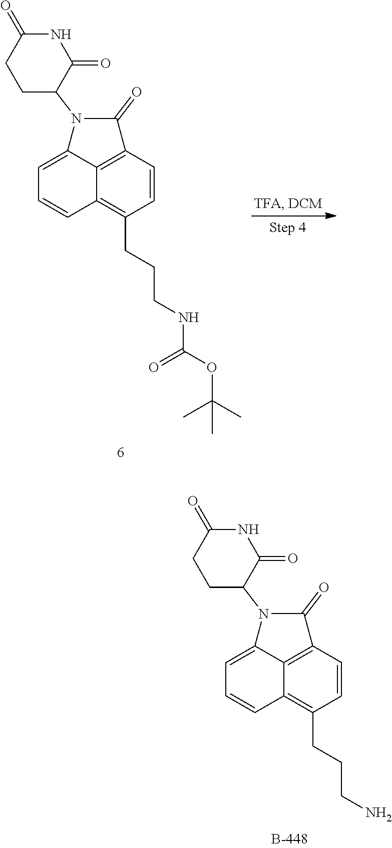 [SMILES: CC(C)(C)OC(=O)NCCCc1ccc2c3c(cccc13)N(C1CCC(=O)NC1=O)C2=O ; NCCCc1ccc2c3c(cccc13)N(C1CCC(=O)NC1=O)C2=O]

### Step 1: tert-butyl N-tert-butoxycarbonyl-N-[3-(2-oxo-1H-benzo[cd]indol-5-yl) prop-2-ynyl]carbamate (3)

Copper (I) iodide (360.82 mg, 1.89 mmol), bis(triphenylphosphine) palladium(II)dichloride (1.33 g, 1.89 mmol) and triethylamine (3.83 g, 37.89 mmol, 5.28 mL) were added to a solution of 5-bromo-1H-benzo[cd]indol-2-one (1, 4.7 g, 18.95 mmol) and tert-butyl N-tert-butoxycarbonyl-N-prop-2-ynyl-carbamate (2, 5.80 g, 22.74 mmol) in THF (20 mL), and the reaction mixture was stirred at 60° C. overnight. The reaction was quenched with water and extracted with ethyl acetate. The organic layer was then dried over anhydrous sodium sulfate, concentrated and purified by flash column chromatography to afford tert-butyl N-tert-butoxycarbonyl-N-[3-(2-oxo-1H-benzo[cd]indol-5-yl) prop-2-ynyl]carbamate (3, 3.5 g, 7.70 mmol, 41% yield) as a yellow solid. LCMS (ES+): m/z 423.2 [M+H]⁺.

### Step 2: tert-butyl N-[3-[1-(2,6-dioxo-3-piperidyl)-2-oxo-benzo[cd]indol-5-yl]prop-2-ynyl]carbamate (5)

To a stirred solution of 3-bromopiperidine-2,6-dione (4, 2.27 g, 11.84 mmol) in THF (50 mL) was added tert-butyl N-tert-butoxycarbonyl-N-[3-(2-oxo-1H-benzo[cd]indol-5-yl) prop-2-ynyl]carbamate (3, 2.5 g, 5.92 mmol) at 0° C. and the mixture was stirred for 10 min. Lithium bis(trimethylsilyl)amide (1.0 M in THF) (1.98 g, 11.84 mmol) was added drop wise under inert condition, and the resulting mixture was stirred at 60° C. for 6 h and at rt for 16 h. After completion, the reaction was cooled to 0° C. and quenched with saturated ammonium chloride solution. The mixture was diluted with cold water and extracted with ethyl acetate. The combined organic layers were washed with brine, dried over anhydrous sodium sulfate, filtered, concentrated and purified by CombiFlash® (30% ethyl acetate in hexane) to afford tert-butyl N—[3-[1-(2,6-dioxo-3-piperidyl)-2-oxo-benzo[cd]indol-5-yl]prop-2-ynyl]carbamate (5, 0.7 g, 1.34 mmol, 23% yield). LCMS (ES+): m/z 434.4 [M+H]⁺.

### Step 3: tert-butyl N-[3-[1-(2,6-dioxo-3-piperidyl)-2-oxo-benzo[cd]indol-5-yl]propyl]carbamate (6)

tert-butyl N-[3-[1-(2,6-dioxo-3-piperidyl)-2-oxo-benzo[cd]indol-5-yl]propyl]carbamate (6, 0.45 g, 897.97 μmol, 56% yield) was synthesized from tert-butyl N-[3-[1-(2,6-dioxo-3-piperidyl)-2-oxo-benzo[cd]indol-5-yl]prop-2-ynyl]carbamate (5, 0.7 g, 1.61 mmol) in a similar fashion to Compound B-6, except using 0.7 g palladium on carbon (10 wt. %). The material was purified by CombiFlash® (40% ethyl acetate in hexane). LCMS (ES⁺): m/z 382.0 [M−tBu+H]⁺.

### Step 4: 3-[5-(3-aminopropyl)-2-oxo-benzo[cd]indol-1-yl]piperidine-2,6-dione (B-448)

3-[5-(3-aminopropyl)-2-oxo-benzo[cd]indol-1-yl]piperidine-2,6-dione (B-448, 430 mg, 762.08 μmol, 67% yield, TFA salt) was synthesized from tert-butyl N-[3-[1-(2,6-dioxo-3-piperidyl)-2-oxo-benzo[cd]indol-5-yl]propyl]carbamate (6, 500 mg, 1.14 mmol) in a similar fashion to Compound A-62, except using 57 eq. TFA. The material was triturated with ether and lyophilized. The material was used in the next step without further characterization.

### 3-[4-chloro-3-methyl-2-oxo-5-(4-piperidyl)benzimidazol-1-yl]piperidine-2,6-dione (B-449)

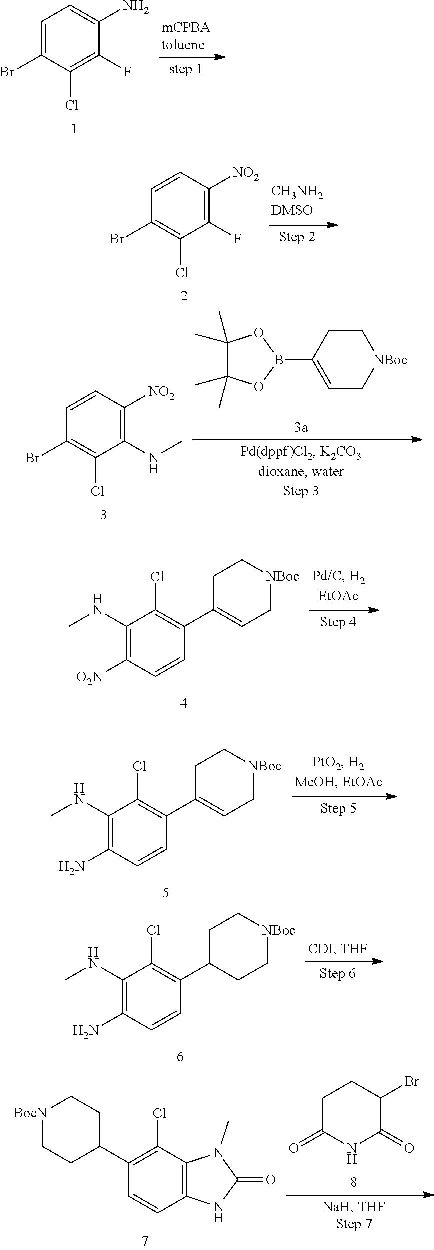

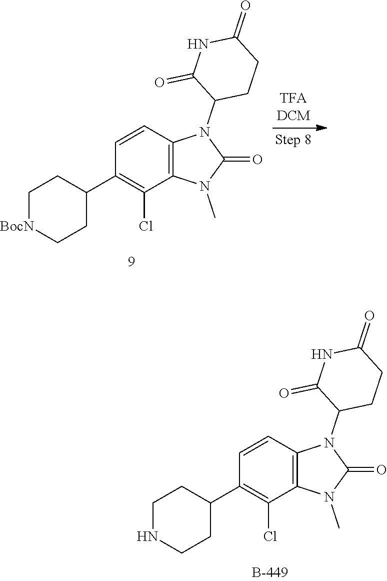 [SMILES: Cn1c(=O)n(C2CCC(=O)NC2=O)c2ccc(C3CCN(C(=O)OC(C)(C)C)CC3)c(Cl)c21 ; Cn1c(=O)n(C2CCC(=O)NC2=O)c2ccc(C3CCNCC3)c(Cl)c21]

### Step 1: 1-bromo-2-chloro-3-fluoro-4-nitrobenzene (2)

To stirred solution of 4-bromo-3-chloro-2-fluoro-aniline (1, 30 g, 133.66 mmol) in Toluene (375 mL), 3-chlorobenzenecarboperoxoic acid (92.26 g, 534.62 mmol) was added slowly at rt. After complete addition of 3-chlorobenzenecarboperoxoic acid (92.26 g, 534.62 mmol), reaction was kept on stirring at 50-55° C. for 16 h. After completion of reaction, Dichloromethane was added to the reaction mixture and then filtered. Filtrate was concentrated and then purified by flash column chromatography to afford 1-bromo-2-chloro-3-fluoro-4-nitrobenzene (2, 7.6 g, 26.77 mmol, 20% yield). The material was used in the next step without further characterization.

### Step 2: 3-bromo-2-chloro-N-methyl-6-nitroaniline (3)

To a stirred solution of 1-bromo-2-chloro-3-fluoro-4-nitrobenzene (2, 7.6 g, 29.87 mmol) in DMSO (15 mL) Methylamine (40% w/w aq. soln.) (4.64 g, 149.35 mmol, 5.16 mL) was added slowly at rt. Reaction mixture was kept on stirring for 2 h. After completion of reaction, the reaction mixture was diluted with ice cold water, where a solid precipitate was formed, which was filtered. The solid was washed with water and dried in vacuo to give 3-bromo-2-chloro-N-methyl-6-nitroaniline (3, 7.5 g, 26.29 mmol, 88% yield). LCMS (ES⁺): m/z 265.09 [M+H]⁺.

### Step 3: tert-butyl 4-(2-chloro-3-(methylamino)-4-nitrophenyl)-3,6-dihydropyridine-1(2H)-carboxylate (4)

To a stirred solution of 3-bromo-2-chloro-N-methyl-6-nitroaniline (3, 7.5 g, 28.25 mmol) in 1,4-Dioxane (50 mL) and Water (50 mL), Potassium carbonate (11.71 g, 84.75 mmol, 5.11 mL) and tert-butyl 4-(4,4,5,5-tetramethyl-1,3,2-dioxaborolan-2-yl)-3,6-dihydropyridine-1(2H)-carboxylate (8.73 g, 28.25 mmol) were added and reaction mixture was purged with Ar for 10-15 min and then DCM, [1,1′-Bis(diphenylphosphino)ferrocene]dichloropalladium(II) (1:1) (1.15 g, 1.41 mmol) was added. The reaction mixture was allowed to stir for 2 h at 100-110° C. under Ar atmosphere. After completion of reaction, it was quenched with brine solution and extracted with ethyl acetate. It was dried over sodium sulfate, concentrated and purified by flash column chromatography to afford tert-butyl 4-(2-chloro-3-(methylamino)-4-nitrophenyl)-3,6-dihydropyridine-1(2H)-carboxy late (4, 9.1 g, 24.10 mmol, 85% yield). LCMS (ES⁺): m/z 368.28 [M+H]⁺.

### Step 4: tert-butyl 4-(4-amino-2-chloro-3-(methylamino)phenyl)-3,6-dihydropyridine-1(2H)-carboxylate (5)

In a round-bottom flask, a solution of tert-butyl 4-(2-chloro-3-(methylamino)-4-nitrophenyl)-3,6-dihydropyridine-1(2H)-carboxylate (4, 500 mg, 1.36 mmol) in ethyl acetate was taken, Palladium, 10% on carbon, Type 487, dry (144.66 mg, 1.36 mmol) was added at rt. Reaction was kept on stirring for 5 h at 10 psi pressure. After completion of reaction, it was filtered through Celite, concentrated, and purified by flash column chromatography to afford tert-butyl 4-(4-amino-2-chloro-3-(methylamino)phenyl)-3,6-dihydropyridine-1(2H)-carboxylate (5, 350 mg, 642.31 μmol, 47% yield, 62% purity).

LCMS (ES⁺): m/z 338.30 [M+H]⁺.

### Step 5: tert-butyl 4-(4-amino-2-chloro-3-(methylamino)phenyl)piperidine-1-carboxylate (6)

In a paar shaker, tert-butyl 4-(4-amino-2-chloro-3-(methylamino)phenyl)-3,6-dihydropyridine-1(2H)-carboxylate (5, 2.68 g, 7.93 mmol) in methanol (50 mL) and ethyl acetate (50 mL) was added Platinum (IV) oxide (1.80 g, 7.93 mmol). After 7 h, the reaction was filtered through Celite and concentrated. It was then purified by flash column chromatography to afford tert-butyl 4-(4-amino-2-chloro-3-(methylamino)phenyl)piperidine-1-carboxylate (6, 1.9 g, 4.91 mmol, 62% yield).

LCMS (ES⁺): m/z 340.31 [M+H]⁺.

### Step 6: tert-butyl 4-(4-chloro-3-methyl-2-oxo-2,3-dihydro-1H-benzo[d]imidazol-5-yl)piperidine-1-carboxylate (7)

ter-butyl 4-(4-chloro-3-methyl-2-oxo-2,3-dihydro-1H-benzo[d]imidazol-5-yl)piperidine-1-carboxylate (7, 4.5 g, 11.70 mmol, 72% yield) was synthesized from tert-butyl 4-(4-amino-2-chloro-3-(methylamino)phenyl)piperidine-1-carboxylate (6, 5.5 g, 16.18 mmol) in a similar fashion to Compound B-208, except using 3 eq. CDI and heating at 65° C. The solvent was removed and the residue diluted with water, washed with n-hexane and dried.

LCMS (ES⁻): m/z 364.23 [M−H]⁻.

### Step 7: tert-butyl 4-(4-chloro-1-(2,6-dioxopiperidin-3-yl)-3-methyl-2-oxo-2,3-dihydro-1H-benzo[d]imidazol-5-yl)piperidine-1-carboxylate (9)

To a solution of Sodium hydride (60% dispersion in mineral oil) (502.71 mg, 21.87 mmol) in THF (150 mL), tert-butyl 4-(4-chloro-3-methyl-2-oxo-2,3-dihydro-1H-benzo[d]imidazol-5-yl)piperidine-1-carboxylate (7, 4 g, 10.93 mmol) was added portionwise at rt. Then reaction mixture was kept on stirring at 60° C. After 30 min, 3-bromopiperidine-2,6-dione (8, 5.25 g, 27.33 mmol) in THF was added slowly and reaction was continued for 5 h at 60° C. After completion of reaction, it was cooled to rt and methanol was added to quenched the reaction. Then it was washed with brine and organic layer was extracted with ethyl acetate. It was dried over anhydrous sodium sulfate, concentrated and purified by flash column chromatography to afford tert-butyl 4-(4-chloro-1-(2,6-dioxopiperidin-3-yl)-3-methyl-2-oxo-2,3-dihydro-1H-benzo[d]imidazol-5-yl)piperidine-1-carboxy late (9, 1.9 g, 3.76 mmol, 34% yield).

LCMS (ES⁻): m/z 475.24 [M−H]⁻.

### Step 8:3-(4-chloro-3-methyl-2-oxo-5-(piperidin-4-yl)-2,3-dihydro-1H-benzo[d]imidazol-1-yl)piperidine-2,6-dione (B-449)

3-(4-chloro-3-methyl-2-oxo-5-(piperidin-4-yl)-2,3-dihydro-1H-benzo[d]imidazol-1-yl)piperidine-2,6-dione (B-449, 50 mg, 94.94 μmol, 91% yield, TFA salt) was synthesized from tert-butyl 4-(4-chloro-1-(2,6-dioxopiperidin-3-yl)-3-methyl-2-oxo-2,3-dihydro-1H-benzo[d]imidazol-5-yl)piperidine-1-carboxy late (9, 50 mg, 104.83 μmol) in a similar fashion to Compound A-62, except using 124 eq. TFA and triturating the product with diethyl ether and hexane. LCMS (ES⁺): m/z 377.21 [M+H]⁺.

### 3-[2-methyl-S-(4-piperidyl) indol-1-yl]piperidine-2,6-dione (B-450)

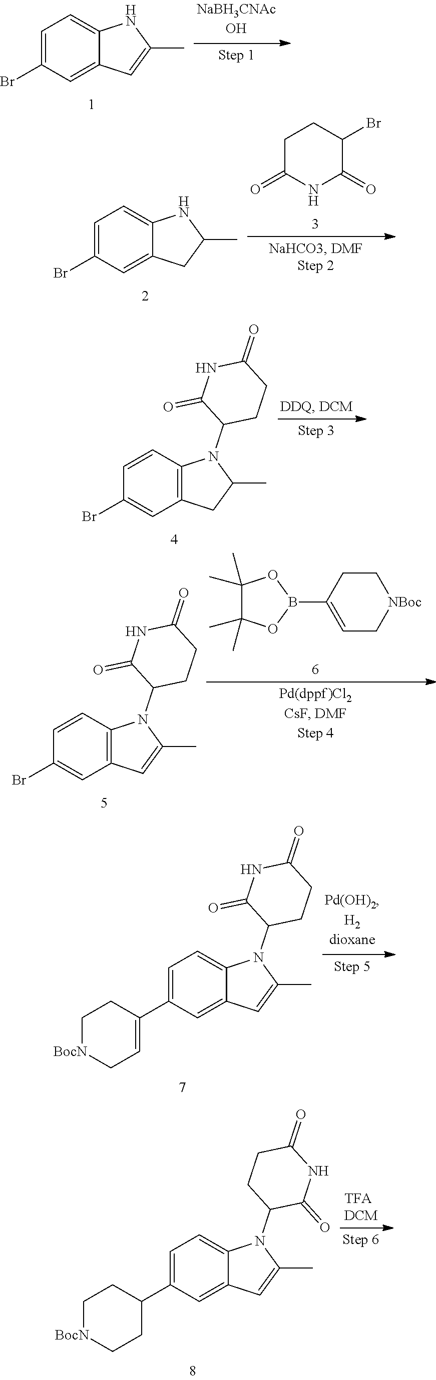

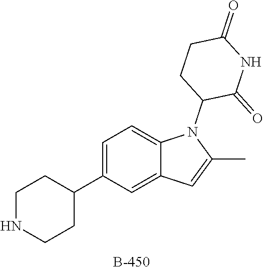 [SMILES: Cc1cc2cc(C3CCNCC3)ccc2n1C1CCC(=O)NC1=O]

### Step 1: 5-bromo-2-methyl-indoline (2)

In a double neck round bottle flask, Acetic acid (200 mL) was purged, to this solution sodium cyanoborohydride (8.97 g, 142.81 mmol) was added slowly in ice cold condition and resulting solution was stirred until bubbling was stopped. 5-bromo-2-methyl-1H-indole (1, 10 g, 47.60 mmol) dissolved in acetic 100 mL was added slowly to the above solution. The reaction mixture was stirred for 12 h. The reaction mixture was neutralized with saturated K₂CO₃ and extracted by ethyl acetate. The organic part was washed with brine water, dried over Na₂SO₄, concentrated and purified by column chromatography using 20% ethyl acetate in hexane as eluent to give 5-bromo-2-methyl-indoline (2, 6 g, 28.29 mmol, 59.43% yield).

LCMS (ES⁺): m/z 214.11 [M+H]⁺.

### Step 2: 3-(5-bromo-2-methyl-indolin-1-yl)piperidine-2,6-dione (4)

To a solution of 5-bromo-2-methyl-indoline (2, 3 g, 14.15 mmol) in DMF (60 mL) taken in a sealed tube, 3-bromopiperidine-2,6-dione (3, 5.43 g, 28.29 mmol) and Sodium bicarbonate (4.75 g, 56.58 mmol, 2.20 mL) was added. The reaction mixture was stirred under heating at 70° C. for 72 h. Reaction mixture was poured into ice water and extracted with ethyl acetate. Organic part was washed with brine water and dried over Na₂SO₄. The organic layer was concentrated and the residue purified by column chromatography using 30% ethyl acetate in hexane as eluent to afford 3-(5-bromo-2-methyl-indolin-1-yl)piperidine-2,6-dione (4, 1.88 g, 5.82 mmol, 41% yield).

LCMS (ES⁺): m/z 323.07 [M+H]⁺.

### Step 3: 3-(5-bromo-2-methyl-indol-1-yl)piperidine-2,6-dione (5)

To a stirred solution of compound 3-(5-bromo-2-methyl-indolin-1-yl)piperidine-2,6-dione (4, 1.88 g, 5.82 mmol) in DCM (60 mL), DDQ (1.32 g, 5.82 mmol) was added slowly in at 0° C. After complete addition, stirring was continued for 1 h at rt. Reaction mixture was extracted with DCM and organic part was washed with 1M NaOH solution. Then organic layer was dried over Na₂SO₄, filtered, concentrated and purified by column chromatography using 40% ethyl acetate in hexane mixture to afford 3-(5-bromo-2-methyl-indol-1-yl)piperidine-2,6-dione (5, 728.77 mg, 2.26 mmol, 39% yield). The material was used in the next step without further characterization.

### Step 4: tert-butyl 4-[1-(2,6-dioxo-3-piperidyl)-2-methyl-indol-5-yl]-3,6-dihydro-2H-pyridine-1-carboxylate (7)

tert-butyl 4-[1-(2,6-dioxo-3-piperidyl)-2-methyl-indol-5-yl]-3,6-dihydro-2H-pyridine-1-carboxylate (7, 265 mg, 300.35 μmol, 24% yield, 48% purity) was synthesized from 3-(5-bromo-2-methyl-indol-1-yl)piperidine-2,6-dione (5, 400 mg, 1.25 mmol) and tert-butyl 4-(4,4,5,5-tetramethyl-1,3,2-dioxaborolan-2-yl)-3,6-dihydro-2H-pyridine-1-carboxylate (6, 577.66 mg, 1.87 mmol) in a similar fashion to Compound B-133, except using 0.1 eq. [1,1′-Bis(diphenylphosphino)ferrocene]dichloropalladium(II) complex with dichloromethane and 2.5 eq. CsF.

LCMS (ES+): m/z 368.2 (M−tBu+H)⁺

### Step 5: tert-butyl 4-[1-(2,6-dioxo-3-piperidyl)-2-methyl-indol-5-yl]piperidine-1-carboxylate (8)

tert-butyl 4-[1-(2,6-dioxo-3-piperidyl)-2-methyl-indol-5-yl]piperidine-1-carboxylate (8, 220 mg, 444.63 μmol, 83% yield) was synthesized from tert-butyl 4-[1-(2,6-dioxo-3-piperidyl)-2-methyl-indol-5-yl]-3,6-dihydro-2H-pyridine-1-carboxylate (7, 265 mg, 538.13 μmol) in a similar fashion to Compound B-103, except using 20 wt. % palladium hydroxide on carbon, 50% water (50 mg, 71.20 μmol) and triturating the product with pet ether. LCMS (ES+): m/z 370.2 (M−tBu+H)⁺

### Step 6: 3-[2-methyl-5-(4-piperidyl) indol-1-yl]piperidine-2,6-dione (B-450)

3-[2-methyl-5-(4-piperidyl) indol-1-yl]piperidine-2,6-dione (B-450, 200 mg, 236.67 μmol, 53% yield, 52% purity, TFA salt) was synthesized from tert-butyl 4-[1-(2,6-dioxo-3-piperidyl)-2-methyl-indol-5-yl]piperidine-1-carboxylate (8, 220 mg, 444.63 μmol) in a similar fashion to Compound A-62, except using 58 eq. TFA and triturating the product with MTBE. LCMS (ES+): m/z 326.2 [M+H]⁺.

### 3-[3-methyl-5-[(2R)-2-methylpiperazin-1-yl]-2-oxo-benzimidazol-1-yl]piperidine-2,6-dione (B-451)

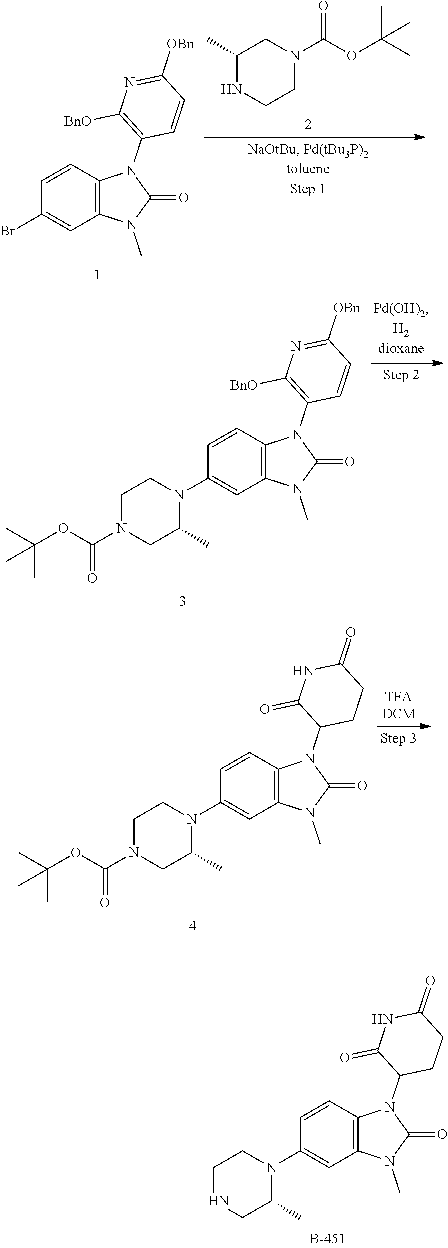

### Step 1: tert-butyl (3R)-4-[1-(2,6-dibenzyloxy-3-pyridyl)-3-methyl-2-oxo-benzimidazol-5-yl]-3-methyl-piperazine-1-carboxylate (3)

To a stirred solution of 5-bromo-1-(2,6-dibenzyloxy-3-pyridyl)-3-methyl-benzimidazol-2-one (1, 6 g, 11.62 mmol) in toluene (20 mL) was added tert-butyl (3R)-3-methylpiperazine-1-carboxylate (2, 2.33 g, 11.62 mmol) and NaOtBu (2.23 g, 23.24 mmol) purged with N2 gas for 10 min. Pd(t-Bu3P)2 (593.80 mg, 1.16 mmol) was added and the mixture purged again for 5 min and then heated at 110° C. for 2 h. The reaction was extracted with EtOAc and water (3×10 mL). The organic layer was washed with brine solution and dried with anhydrous sodium sulfate and filtered, concentrated and purified by column chromatography using 100-200 mesh silica gel with 10-50% EA/PE to afford tert-butyl (3R)-4-[1-(2,6-dibenzyloxy-3-pyridyl)-3-methyl-2-oxo-benzimidazol-5-yl]-3-methyl-piperazine-1-carboxylate (3, 3.5 g, 5.45 mmol, 47% yield) as a light brown colored solid.

LCMS (ES⁺): m/z 636.47 [M+H]⁺.

### Step 2: tert-butyl (3R)-4-[1-(2,6-dioxo-3-piperidyl)-3-methyl-2-oxo-benzimidazol-5-yl]-3-methyl-piperazine-1-carboxylate (4)

tert-butyl (3R)-4-[1-(2,6-dioxo-3-piperidyl)-3-methyl-2-oxo-benzimidazol-5-yl]-3-methyl-piperazine-1-carboxy late (4, 1.0 g, 2.14 mmol, 68% yield) was synthesized from tert-butyl (3R)-4-[1-(2,6-dibenzyloxy-3-pyridyl)-3-methyl-2-oxo-benzimidazol-5-yl]-3-methyl-piperazine-1-carboxylate (3, 2 g, 3.15 mmol) is a similar fashion to Compound B-103, except using 20 wt. % Pd(OH)₂ (441.79 mg, 3.15 mmol). LCMS (ES⁺): m/z 458.36 [M+H]⁺.

### Step 3: 3-[3-methyl-5-[(2R)-2-methylpiperazin-1-yl]-2-oxo-benzimidazol-1-yl]piperidine-2,6-dione (B-451)

3-[3-methyl-5-[(2R)-2-methylpiperazin-1-yl]-2-oxo-benzimidazol-1-yl]piperidine-2,6-dione (B-451, 0.6 g, 1.25 mmol, 57.12% yield, TFA salt) was synthesized from tert-butyl (3R)-4-[1-(2,6-dioxo-3-piperidyl)-3-methyl-2-oxo-benzimidazol-5-yl]-3-methyl-piperazine-1-carboxylate (4, 1.0 g, 2.19 mmol) in a similar fashion to Compound A-62, except using 7 eq. TFA and triturating the product with diethyl ether. LCMS (ES⁺): m/z 358.37 [M+H]⁺.

### 3-[3-methyl-5-[(2S)-2-methylpiperazin-1-yl]-2-oxo-benzimidazol-1-yl]piperidine-2,6-dione (B-452)

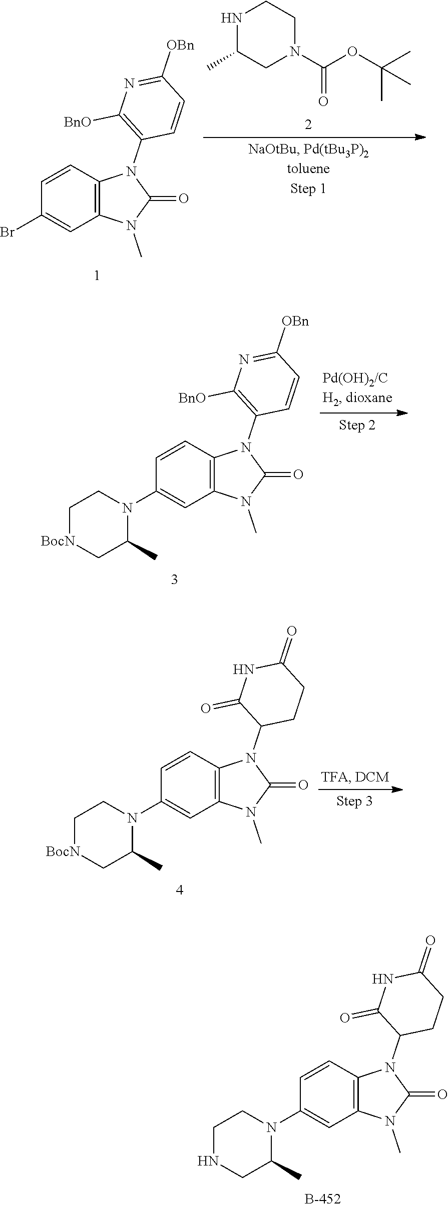

### Step 1: tert-butyl (3S)-4-[1-(2,6-dibenzyloxy-3-pyridyl)-3-methyl-2-oxo-benzimidazol-5-yl]-3-methyl-piperazine-1-carboxylate (3)

To a stirred solution of 5-bromo-1-(2,6-dibenzyloxy-3-pyridyl)-3-methyl-benzimidazol-2-one (1, 5 g, 9.68 mmol) in Toluene (20 mL) add tert-butyl (3S)-3-methylpiperazine-1-carboxylate (2, 1.94 g, 9.68 mmol) and sodium tert-butoxide (1.86 g, 19.37 mmol) purged with N₂ gas for 10 min and Pd(t-Bu3P)₂ (494.84 mg, 968.27 μmol) added again purged for 5 min. The reaction was stirred at 110° C. for 2 h. The reaction mixture was cooled to rt, diluted with water and extracted with EtOAc. Organic layer was dried with anhydrous sodium sulfate, concentrated and purified by column chromatography using silica 100-200 mesh with 10-50% EA in PE to afford tert-butyl (3S)-4-[1-(2,6-dibenzyloxy-3-pyridyl)-3-methyl-2-oxo-benzimidazol-5-yl]-3-methyl-piperazine-1-carboxylate (3, 3 g, 2.39 mmol, 25% yield, 51% purity) LCMS (ES⁺): m/z 636.39 [M+H]⁺.

### Step 2: tert-butyl (3S)-4-[1-(2,6-dioxo-3-piperidyl)-3-methyl-2-oxo-benzimidazol-5-yl]-3-methyl-piperazine-1-carboxylate (4)

tert-butyl (3S)-4-[1-(2,6-dioxo-3-piperidyl)-3-methyl-2-oxo-benzimidazol-5-yl]-3-methyl-piperazine-1-carboxylate (4, 1.3 g, 2.64 mmol, 56% yield) was synthesized from tert-butyl (3S)-4-[1-(2,6-dibenzyloxy-3-pyridyl)-3-methyl-2-oxo-benzimidazol-5-yl]-3-methyl-piperazine-1-carboxylate (3, 3.0 g, 4.72 mmol) in a similar fashion to Compound B-103, except using 20 wt. % Pd(OH)₂ (2.7 g, 19.23 mmol). LCMS (ES⁺): m/z 458.32 [M+H]⁺.

### Step 3: 3-[3-methyl-5-[(2S)-2-methylpiperazin-1-yl]-2-oxo-benzimidazol-1-yl]piperidine-2,6-dione (B-452)

3-[3-methyl-5-[(2S)-2-methylpiperazin-1-yl]-2-oxo-benzimidazol-1-yl]piperidine-2,6-dione (B-452, 0.98 g, 2.01 mmol, 71% yield, TFA salt) was synthesized from tert-butyl (3S)-4-[1-(2,6-dioxo-3-piperidyl)-3-methyl-2-oxo-benzimidazol-5-yl]-3-methyl-piperazine-1-carboxylate (4, 1.3 g, 2.84 mmol) in a similar fashion to Compound A-62, except using 5 eq. TFA and triturating the product with diethyl ether. LCMS (ES⁺): m/z 358.37 [M+H]⁺.

### 3-[3-methyl-5-(4-piperidyl) indol-1-yl]piperidine-2,6-dione (B-453)

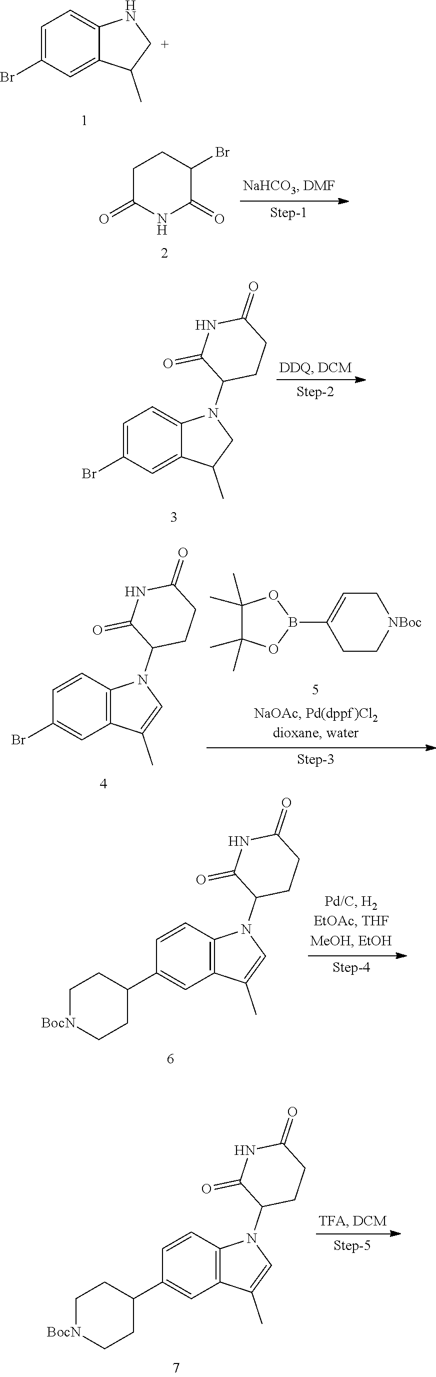

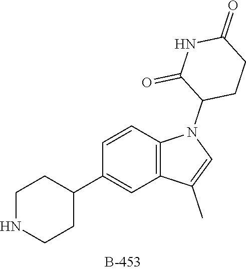 [SMILES: Cc1cn(C2CCC(=O)NC2=O)c2ccc(C3CCNCC3)cc12]

### Step 1: 3-(5-bromo-3-methyl-indolin-1-yl)piperidine-2,6-dione (3)

To a solution of 5-bromo-3-methyl-indoline (1, 5.5 g, 25.93 mmol) in DMF (70 mL) in a sealed tube, was added 3-bromopiperidine-2,6-dione (2, 7.47 g, 38.90 mmol) and sodium bicarbonate (6.54 g, 77.80 mmol, 3.03 mL). The reaction mixture was stirred under beating at 70° C. for 48 h, then poured into ice water and extracted with ethyl acetate. The organic layers were washed with brine water, dried over Na₂SO₄, concentrated and purified by column chromatography using 30% ethyl acetate in hexane as eluent to afford 3-(5-bromo-3-methyl-indolin-1-yl)piperidine-2,6-dione (3, 2.5 g, 7.43 mmol, 29% yield). LCMS (ES⁺): m/z 323.26 [M+H]⁺.

### Step 2: 3-(5-bromo-3-methyl-indol-1-yl)piperidine-2,6-dione (4)

To a stirred solution of compound 3-(5-bromo-3-methyl-indolin-1-yl)piperidine-2,6-dione (3, 2.5 g, 7.74 mmol) in DCM (80 mL) was added DDQ (2.11 g, 9.28 mmol) slowly at 0° C. After addition, stirring was continued for 1 h at rt. After product formation was confirmed by LCMS, the reaction mixture was extracted with DCM and the organic layers washed with 1M NaOH. Then the organic layer was dried over Na₂SO₄, concentrated and purified by column chromatography (silica gel, 40% ethyl acetate in hexane) to afford 3-(5-bromo-3-methyl-indol-1-yl)piperidine-2,6-dione (4, 911.38 mg, 2.83 mmol, 37% yield). LCMS (ES⁺): m/z 321.11 [M+H]⁺.

### Step 3: tert-butyl 4-[1-(2,6-dioxo-3-piperidyl)-3-methyl-indol-5-yl]-3,6-dihydro-2H-pyridine-1-carboxylate (6)

3-(5-bromo-3-methyl-indol-1-yl)piperidine-2,6-dione (4, 0.05 g, 155.68 μmol) was charged into a 250 mL round bottom flask and solvated in 1,4-dioxane (2 mL) and water (0.2 mL). Tert-butyl 4-(4,4,5,5-tetramethyl-1,3,2-dioxaborolan-2-yl)-3,6-dihydro-2H-pyridine-1-carboxylate (5, 62.58 mg, 202.39 μmol) and sodium acetate (38.31 mg, 467.04 μmol) were added at rt under argon gas. The reaction mixture was degassed for 20 with argon min. After degassing, cyclopentyl(diphenyl)phosphane; dichloropalladium; iron (11.39 mg, 15.57 μmol) was added and the reaction was heated at 80° C. for 6 h. The mixture was filtered through Celite and washed with ethyl acetate (10 mL×3). The filtrate was concentrated and purified by column chromatography (silica gel 100-200 mesh, 0-50% ethyl acetate in pet ether) to afford tert-butyl 4-[1-(2,6-dioxo-3-piperidyl)-3-methyl-indol-5-yl]-3,6-dihydro-2H-pyridine-1-carboxylate (6, 0.04 g, 49.11 μmol, 32% yield) as a grey colored solid. LCMS (ES⁻): m/z 422.51 [M−H]⁻.

### Step 4: tert-butyl 4-[1-(2,6-dioxo-3-piperidyl)-3-methyl-indol-5-yl]piperidine-1-carboxylate (7)

Tert-butyl 4-[1-(2,6-dioxo-3-piperidyl)-3-methyl-indol-5-yl]-3,6-dihydro-2H-pyridine-1-carboxylate (6, 0.1 g, 236.13 μmol) was charged into a round-bottom flask and solvated in ethyl acetate (2 mL). To this stirring solution was added palladium, 10% on carbon, type 487, dry (25.13 mg, 236.13 μmol), then H2 pressure was applied from a bladder and the reaction was stirred continuously at rt for 16 h. The reaction mixture was filtered through a Celite, washed with ethyl acetate (10 mL) and methanol (10 mL)/The filtrate was concentrated, triturated with n-pentane (5 mL) and concentrated under reduced pressure to obtain tert-butyl 4-[1-(2,6-dioxo-3-piperidyl)-3-methyl-indol-5-yl]piperidine-1-carboxylate (7, 0.08 g, 131.60 μmol, 56% yield) as a grey color solid.

LCMS (ES⁻): m/z 424.34 [M−H]⁻.

### Step 5: 3-[3-methyl-5-(4-piperidyl) indol-1-yl]piperidine-2,6-dione (B-453)

3-[3-methyl-5-(4-piperidyl) indol-1-yl]piperidine-2,6-dione (B-453, 0.015 g, 29.36 μmol, 42% yield, TFA salt) was synthesized from ter-butyl 4-[1-(2,6-dioxo-3-piperidyl)-3-methyl-indol-5-yl]piperidine-1-carboxylate (7, 0.03 g, 70.50 μmol) in a similar fashion to Compound A-62, except using 5 eq. TFA and triturating the product with diethyl ether. LCMS (ES+): m/z 326.35 [M+H]⁺.

### 2-[4-[[1-(2,6-dioxo-3-piperidyl)-3-methyl-2-oxo-benzimidazol-5-yl]methyl]phenyl]acetic acid (B-454)

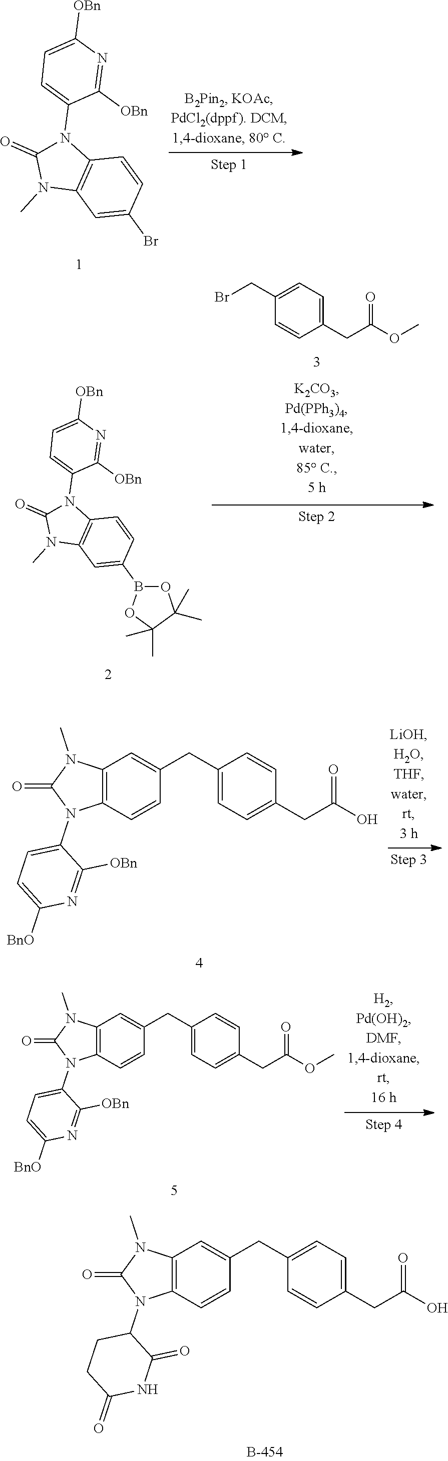 [SMILES: Cn1c(=O)n(-c2ccc(OCc3ccccc3)nc2OCc2ccccc2)c2ccc(Br)cc21 ; COC(=O)Cc1ccc(CBr)cc1 ; Cn1c(=O)n(-c2ccc(OCc3ccccc3)nc2OCc2ccccc2)c2ccc(B3OC(C)(C)C(C)(C)O3)cc21 ; Cn1c(=O)n(-c2ccc(OCc3ccccc3)nc2OCc2ccccc2)c2ccc(Cc3ccc(CC(=O)O)cc3)cc21 ; COC(=O)Cc1ccc(Cc2ccc3c(c2)n(C)c(=O)n3-c2ccc(OCc3ccccc3)nc2OCc2ccccc2)cc1 ; Cn1c(=O)n(C2CCC(=O)NC2=O)c2ccc(Cc3ccc(CC(=O)O)cc3)cc21]

### Step 1: 1-(2,6-dibenzyloxy-3-pyridyl)-3-methyl-5-(4,4,5,5-tetramethyl-1,3,2-dioxaborolan-2-yl)benzimidazol-2-one (2)

Into a 50 mL pressure tube containing a well-stirred solution of 5-bromo-1-(2,6-dibenzyloxy-3-pyridyl)-3-methyl-benzimidazol-2-one (1, 1 g, 1.88 mmol) in anhydrous 1,4-dioxane (8 mL) was added potassium acetate (553.06 mg, 5.64 mmol), bis(pinacolato)diboron (1.43 g, 5.64 mmol) and Pd(dppf)Cl₂·DCM (153.40 mg, 187.84 μmol) at ambient temperature under nitrogen atmosphere. The resulting mixture was degassed by bubbling nitrogen gas into the reaction mixture for 10 min. The reaction mixture was heated to 100° C. After 16 h, the reaction mixture was filtered through Celite and washed with ethyl acetate (500 mL). The filtrate layer was washed with water (300 mL) and brine (200 mL), dried over anhydrous Na₂SO₄ and filtered. The filtrate was concentrated under and purified by flash silica-gel column chromatography (0-35% Ethyl acetate in pet ether) to afford 1-(2,6-dibenzyloxy-3-pyridyl)-3-methyl-5-(4,4,5,5-tetramethyl-1,3,2-dioxaborolan-2-yl)benzimidazol-2-one (2, 1 g, 1.67 mmol, 89% yield) as an off-white semisolid.

LCMS (ES+): m/z 564.2 [M+H]⁺.

### Step 2: methyl 2-[4-[[1-(2,6-dibenzyloxy-3-pyridyl)-3-methyl-2-oxo-benzimidazol-5-yl]methyl]phenyl]acetate (4)

Into a 50 mL pressure tube containing a well-stirred solution of 1-(2,6-dibenzyloxy-3-pyridyl)-3-methyl-5-(4,4,5,5-tetramethyl-1,3,2-dioxaborolan-2-yl)benzimidazol-2-one (2, 1 g, 1.67 mmol) and methyl 2-[4-(bromomethyl)phenyl]acetate (3, 1.62 g, 6.67 mmol) in 1,4-dioxane (6 mL) and water (1.2 mL) were added potassium carbonate (691.70 mg, 5.00 mmol) and bis(triphenylphosphine) palladium(II)dichloride (234.20 mg, 333.66 μmol). The reaction mixture was degassed by bubbling nitrogen gas for 10 min and heated at 90° C. After 16 h, the reaction mixture was filtered and washed thoroughly with ethyl acetate (200 mL). The filtrate was concentrated and purified by flash silica gel column chromatography (20-60% ethyl acetate in pet ether) to afford methyl 2-[4-[[1-(2,6-dibenzyloxy-3-pyridyl)-3-methyl-2-oxo-benzimidazol-5-yl]methyl]phenyl]acetate (4, 370 mg, 524.45 μmol, 31% yield) as a brown color liquid.

UP-LCMS (ES+): m/z 600.3 [M+H]⁺.

### Step 3: 2-[4-[[1-(2,6-dibenzyloxy-3-pyridyl)-3-methyl-2-oxo-benzimidazol-5-yl]methyl]phenyl]acetic acid (5)

2-[4-[[1-(2,6-dibenzyloxy-3-pyridyl)-3-methyl-2-oxo-benzimidazol-5-yl]methyl]phenyl]acetic acid (5, 330 mg, 478.73 μmol, 91% yield) was synthesized from methyl 2-[4-[1-(2,6-dibenzyloxy-3-pyridyl)-3-methyl-2-oxo-benzimidazol-5-yl]methyl]phenyl]acetate (4, 370 mg, 524.45 μmol) in a similar fashion to Compound A-9, except using 7 eq. LiOH monohydrate. After acidifying, the aqueous layer was extracted with ethyl acetate (2×100 mL). The combined organic layers were washed with water (100 mL) followed by brine solution (100 mL), dried over anhydrous sodium sulfate, filtered, and dried under vacuum. LCMS (ES+): m/z 586.2 [M+H]⁺.

### Step 4: 2-[4-[[1-(2,6-dioxo-3-piperidyl)-3-methyl-2-oxo-benzimidazol-5-yl]methyl]phenyl]acetic acid (B-454)

2-[4-[[1-(2,6-dioxo-3-piperidyl)-3-methyl-2-oxo-benzimidazol-5-yl]methyl]phenyl]acetic acid (6, 180 mg, 437.05 μmol, 92% yield) was synthesized from 2-[4-[[1-(2,6-dibenzyloxy-3-pyridyl)-3-methyl-2-oxo-benzimidazol-5-yl]methyl]phenyl]acetic acid (B-454, 330 mg, 473.32 μmol) in a similar fashion to Compound B-103, except using 20 wt. % palladium hydroxide on carbon (332.35 mg, 473.32 μmol). LCMS (ES+): m/z 408.2 [M+H]⁺.

### Degrader Synthesis:

### Example 1

### 3-(carboxymethoxy)-4-chloro-5-(3-((1-((3-(6-(4-(((2-(2,6-dioxopiperidin-3-yl)-1,3-dioxoisoindolin-4-yl)amino)methyl)-1H-1,2,3-triazol-1-yl) hexanamido)benzyl)sulfonyl)piperidin-4-yl)amino)phenyl)thiophene-2-carboxylic acid (1)

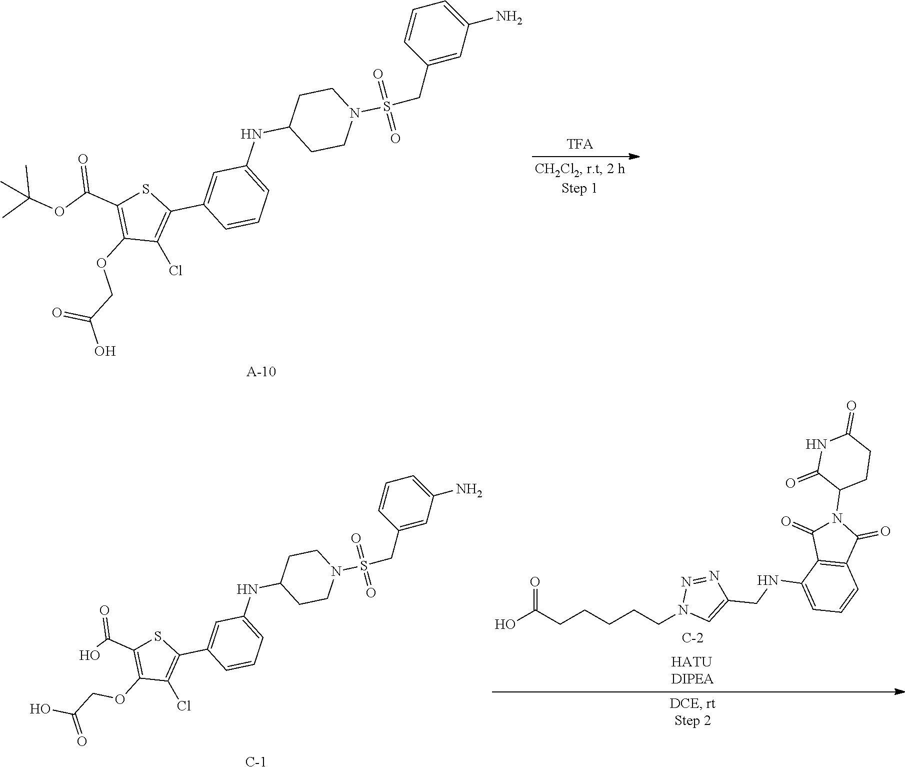

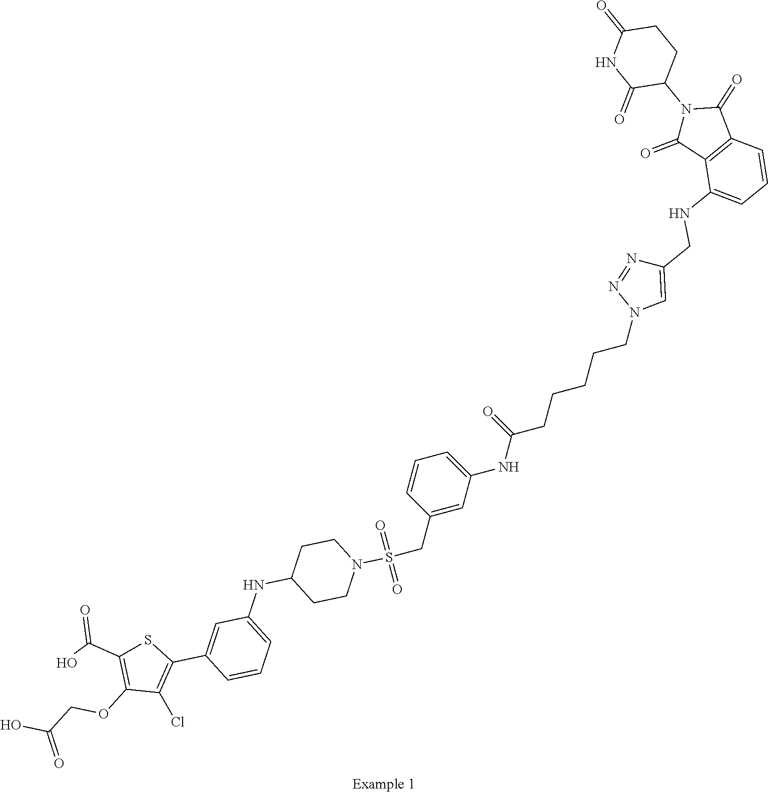 [SMILES: O=C(O)COc1c(C(=O)O)sc(-c2cccc(NC3CCN(S(=O)(=O)Cc4cccc(NC(=O)CCCCCn5cc(CNc6cccc7c6C(=O)N(C6CCC(=O)NC6=O)C7=O)nn5)c4)CC3)c2)c1Cl ; "Example 1"]

### Step 1: 5-[3-[[1-[(3-aminophenyl)methylsulfonyl]-4-piperidyl]amino]phenyl]-3-(carboxymethoxy)-4-chloro-thiophene-2-carboxylic acid (C-1)

Into a 25 mL single neck round bottom flask containing a well-stirred solution of 2-[[5-[3-[[[1-(3-aminophenyl)methylsulfonyl]-4-piperidyl]amino]phenyl]-2-tert-butoxycarbonyl-4-chloro-3-thienyl]oxy]acetic acid (A-10, 200 mg, 314.38 μmol) in anhydrous DCM (5 mL) was added TFA (2.22 g, 19.47 mmol, 1.5 mL) at 0° C. under a nitrogen atmosphere. The reaction mixture was stirred at room temperature for 2 h. The volatiles were removed under reduced pressure and azeotroped with toluene to afford 5-[3-[[1-[(3-aminophenyl)methylsulfonyl]-4-piperidyl]amino]phenyl]-3-(carboxymethoxy)-4-chloro-thiophene-2-carboxylic acid (C-1, 180 mg, 217.47 μmol, 69% yield) as an off-white solid. LCMS (ES+): m/z 579.8 [M+H]⁺.

### Step 2: 3-(carboxymethoxy)-4-chloro-5-(3-((1-((3-(6-(4-(((2-(2,6-dioxopiperidin-3-yl)-1,3-dioxoisoindolin-4-yl)amino)methyl)-1H-1,2,3-triazol-1-yl) hexanamido)benzyl)sulfonyl)piperidin-4-yl)amino)phenyl)thiophene-2-carboxylic acid (1)

6-[4-[[2-(2,6-dioxo-3-piperidyl)-1,3-dioxo-isoindolin-4-yl]amino]methyl]triazol-1-yl]hexanoic acid (C-2, 24.23 mg, 51.72 μmol) in DCE (600 μL) was treated with HATU (23.60 mg, 62.06 μmol) and DIPEA (33.42 mg, 258.59 μmol, 45.04 μL). After 20 min, 5-[3-[[1-[(3-aminophenyl)methylsulfonyl]-4-piperidyl]amino]phenyl]-3-(carboxymethoxy)-4-chloro-thiophene-2-carboxylic acid (C-1, 30 mg, 51.72 μmol) was added, and the reaction stirred at room temperature. Upon completion, the solvent was removed and the product purified by reverse phase chromatography, eluting with 5-100% MeCN in water (with 0.1% TFA modifier) to give 3-(carboxymethoxy)-4-chloro-5-(3-((1-((3-(6-(4-(((2-(2,6-dioxopiperidin-3-yl)-1,3-dioxoisoindolin-4-yl)amino)methyl)-1H-1,2,3-triazol-1-yl) hexanamido)benzyl)sulfonyl)piperidin-4-yl)amino)phenyl)thiophene-2-carboxylic acid (1, 26.7 mg, 25.13 μmol, 49% yield). LCMS (ESI): 1030.733 [M+H]⁺. ¹H NMR (400 MHz, DMSO-d₆) δ 11.08 (s, 1H), 9.92 (s, 1H), 8.00 (s, 1H), 7.68-7.64 (m, 1H), 7.60-7.53 (m, 2H), 7.29 (t, J=7.9 Hz, 1H), 7.21 (t, J=7.9 Hz, 1H), 7.15 (d, J=8.6 Hz, 1H), 7.06 (dd, J=9.6, 7.2 Hz, 2H), 6.91-6.88 (m, 1H), 6.86-6.82 (m, 1H), 6.74-6.69 (m, 1H), 5.05 (dd, J=12.9, 5.4 Hz, 1H), 4.92 (s, 2H), 4.57 (s, 2H), 4.35 (s, 2H), 4.31 (t, J=7.1 Hz, 2H), 3.58-3.50 (m, 2H), 3.47-3.37 (m, 1H), 3.01-2.91 (m, 2H), 2.90-2.82 (m, 1H), 2.63-2.52 (m, 1H), 2.28 (t, J=7.4 Hz, 2H), 2.06-1.99 (m, 1H), 1.98-1.91 (m, 2H), 1.85-1.75 (m, 2H), 1.65-1.55 (m, 2H), 1.44-1.32 (m, 2H), 1.31-1.19 (m, 3H).

### 6-[4-[[[2-(2,6-dioxo-3-piperidyl)-1,3-dioxo-isoindolin-4-yl]amino]methyl]triazol-1-yl]hexanoic acid (C-2)

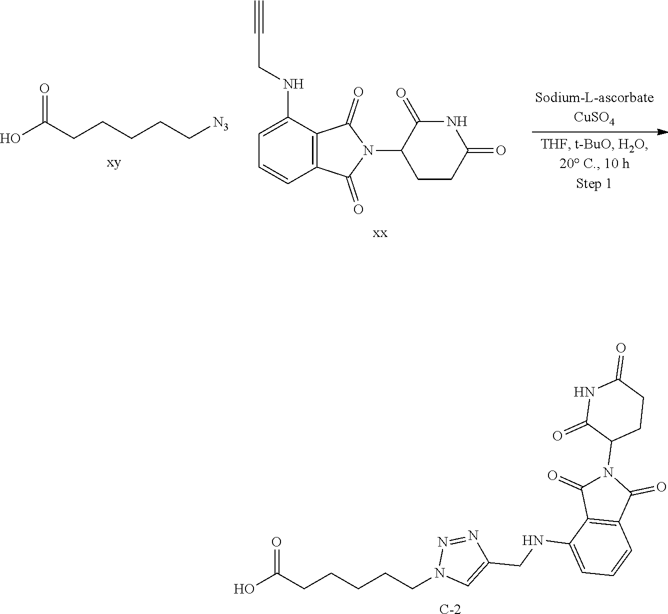

### Step 1: 6-[4-[[[2-(2,6-dioxo-3-piperidyl)-1,3-dioxo-isoindolin-4-yl]amino]methyl]triazol-1-yl]hexanoic acid (C-2)

A mixture of 6-azidohexanoic acid (xy, 9.50 g, 60.4 mmol), 2-(2,6-dioxopiperidin-3-yl)-4-(prop-2-yn-1-ylamino)isoindoline-1,3-dione (xx, 15.7 g, 50.4 mmol), sodium-L-ascorbate (3.99 g, 20.2 mmol) and copper(II) sulfate (1.61 g, 10.1 mmol) in tBuOH (40.0 mL), water (40.0 mL) and THF (40.0 mL) was stirred at room temperature for 10 h. Upon completion, the mixture was concentrated under reduced pressure and the residue was purified by reversed phase HPLC (using 0.1% TFA modifier) to afford 6-[4-[[[2-(2,6-dioxo-3-piperidyl)-1,3-dioxo-isoindolin-4-yl]amino]methyl]triazol-1-yl]hexanoic acid (C-2, 10.4 g, 22.2 mmol, 44% yield) as a yellow solid. LCMS (ES+): m/z 469.2 [M+H]⁺.

### 3-[4-[[[2-(2,6-dioxo-3-piperidyl)-1,3-dioxo-isoindolin-4-yl]amino]methyl]triazol-1-yl]propanoic acid (C-3)

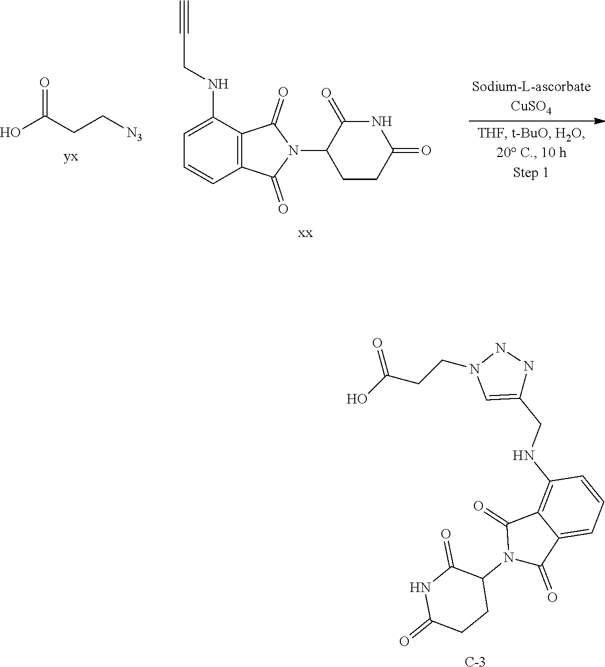

### Step 1: 3-[4-[[2-(2,6-dioxo-3-piperidyl)-1,3-dioxo-isoindolin-4-yl]amino]methyl]triazol-1-yl]propanoic acid (C-3)

3-[4-[[[2-(2,6-dioxo-3˜piperidyl)-1,3-dioxo-isoindolin-4-yl]amino]methyl]triazol-1-yl]propanoic acid (C-3, 12.0 g, 28.1 mmol, 78% yield) was synthesized from 3-azidopropanoic acid (yx) and 2-(2,6-dioxopiperidin-3-yl)-4-(prop-2-yn-1-ylamino)isoindoline-1,3-dione (xx) in a similar fashion to Compound C-2. LCMS (ES+): m/z 427.2 [M+H]⁺.

### Example 2

### 3-(carboxymethoxy)-4-chloro-5-[3-[[1-[[3-[3-[4-[[[2-(2,6-dioxo-3-piperidyl)-1,3-dioxo-isoindolin-4-yl]amino]methyl]triazol-1-yl]propanoylamino]phenyl]methylsulfonyl]-4-piperidyl]amino]phenyl]thiophene-2-carboxylic acid (2)

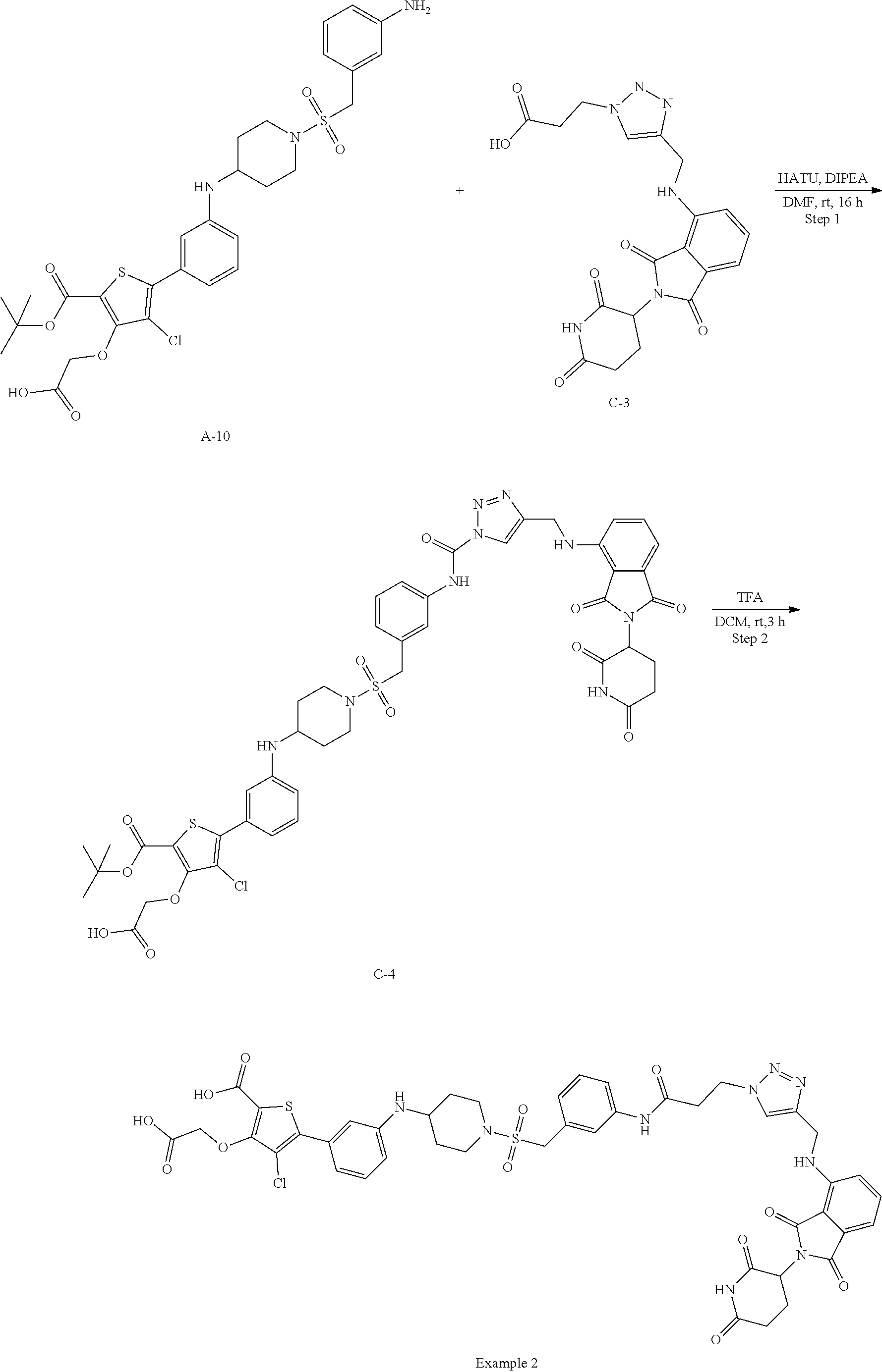

### Step 1: 2-[[2-tert-butoxycarbonyl-4-chloro-5-[3-[[1-[[3-[3-[4-[[[2-(2,6-dioxo-3-piperidyl)-1,3-dioxo-isoindolin-4-yl]amino]methyl]triazol-1-yl]propanoylamino]phenyl]methylsulfonyl]-4-piperidyl]amino]phenyl]-3-thienyl]oxy]acetic acid (C-4)

2-[2-tert-butoxycarbonyl-4-chloro-5-[3-[[1-[[3-[3-[4-[[2-(2,6-dioxo-3-piperidyl)-1,3-dioxo-isoindolin-4-yl]amino]methyl]triazol-1-yl]propanoylamino]phenyl]methylsulfonyl]-4-piperidyl]amino]phenyl]-3-thienyl]oxy]acetic acid (C-4, 60 mg, 49.40 μmol, 22% yield, TFA salt) was synthesized from 3-[4-[[[2-(2,6-dioxo-3-piperidyl)-1,3-dioxo-isoindolin-4-yl]amino]methyl]triazol-1-yl]propanoic acid (C-3) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1 except using DMF as the solvent. The residue was purified by reverse phase preparative HPLC [Prep method: Column: SunFire C18 (19×150 mm) 5 μm, Mobile phase A: 0.1% TFA in water; Mobile phase B: Acetonitrile]. UPLC-MS (ES+): m/z 1044.5 [M+H]⁺.

### Step 2: 3-(carboxymethoxy)-4-chloro-5-[3-[[1-[[3-[3-[4-[[[2-(2,6-dioxo-3-piperidyl)-1,3-dioxo-isoindolin-4-yl]amino]methyl]triazol-1-yl]propanoylamino]phenyl]methylsulfonyl]-4-piperidyl]amino]phenyl]thiophene-2-carboxylic acid (2)

3-(carboxymethoxy)-4-chloro-5-[3-[[1-[[3-[3-[4-[[[2-(2,6-dioxo-3-piperidyl)-1,3-dioxo-isoindolin-4-yl]amino]methyl]triazol-1-yl]propanoylamino]phenyl]methylsulfonyl]-4-piperidyl]amino]phenyl]thiophene-2-carboxylic acid (2, 19.98 mg, 19.30 μmol, 34% yield, TFA salt) was synthesized from 2-[[2-tert-butoxycarbonyl-4-chloro-5-[3-[[1-[[3-[3-[4-[[2-(2,6-dioxo-3-piperidyl)-1,3-dioxo-isoindolin-4-yl]amino]methyl]triazol-1-yl]propanoylamino]phenyl]methylsulfonyl]-4-piperidyl]amino]phenyl]-3-thienyl]oxy]acetic acid (C-4) in a similar fashion to Compound C-1, except using 6 eq. TFA. The residue was purified by reverse phase preparative HPLC [Prep method: Column: SunFire C18 (19×150 mm) 5 μm, Mobile phase A: 0.1% TFA in water: Mobile phase B: Acetonitrile]. UPLC-MS (ES+): m/z 988.4 [M+H]⁺. ¹H NMR (400 MHz, DMSO-d₆) δ 11.10 (s, 1H), 10.11 (s, 1H), 7.99 (s, 1H), 7.60 (s, 1H), 7.56-7.48 (m, 2H), 7.30 (t, J=7.9 Hz, 1H), 7.21 (t, J=7.9 Hz, 1H), 7.14 (d, J=8.6 Hz, 1H), 7.11-7.04 (m, 2H), 7.02 (d, J=7.1 Hz, 1H), 6.88 (d, J=2.7 Hz, 1H), 6.82 (d, J=7.6 Hz, 1H), 6.71 (d, J=8.2 Hz, 1H), 5.05 (dd, J=12.9, 5.3 Hz, 1H), 4.92 (s, 2H), 4.62 (t, J=6.7 Hz, 2H), 4.59-4.54 (m, 2H), 4.36 (s, 2H), 3.59-3.50 (m, 2H), 3.46-3.36 (m, 1H), 3.01-2.81 (m, 5H), 2.63-2.54 (m, 2H), 2.06-1.91 (m, 3H), 1.45-1.29 (m, 2H).

### Example 3

### 3-(carboxymethoxy)-4-chloro-5-[3-[[1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-4-piperidyl]amino]phenyl]thiophene-2-carboxylic acid (3)

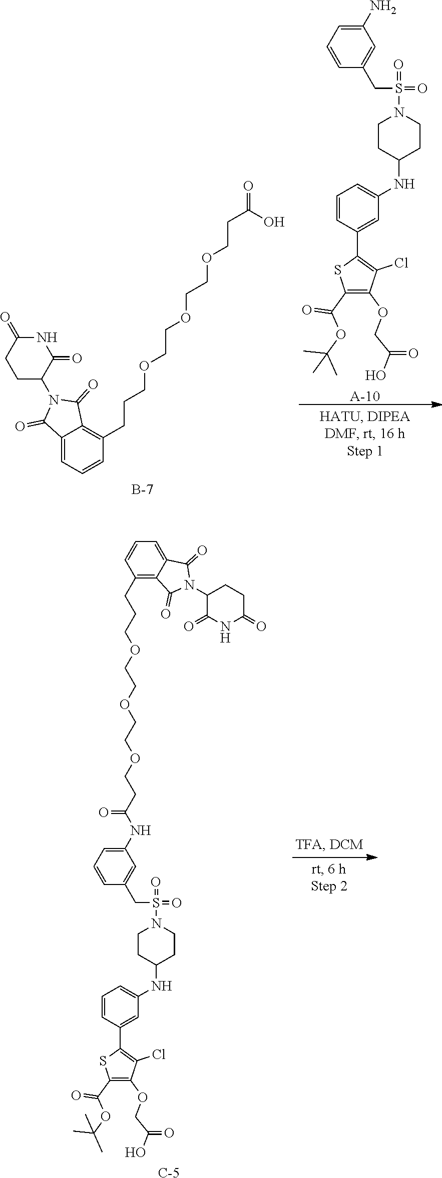

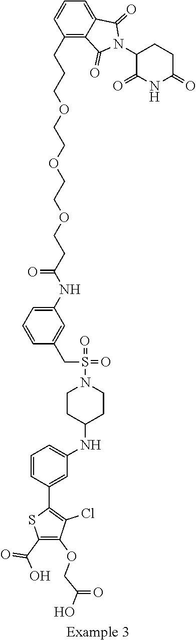

### Step 1: 2-[[2-tert-butoxycarbonyl-4-chloro-5-[3-[[1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-4-piperidyl]amino]phenyl]-3-thienyl]oxy]acetic acid (C-5)

2-[[2-tert-butoxycarbonyl-4-chloro-5-[3-[[1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-4-piperidyl]amino]phenyl]-3-thienyl]oxy]acetic acid (C-5, 21 mg, 19.18 μmol, 10% yield, TFA salt) was synthesized from 3-[2-[2-[3-[2-(2,6-dioxo-3-piperidyl)-1,3-dioxo-isoindolin-4-yl]propoxy]ethoxy]ethoxy]propanoic acid (B-7) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1 except using DMF as the solvent. The residue was purified by reverse phase preparative HPLC [Purification method: Column: SunFire-C18 100 mm, Mobile phase A: 0.1% TFA in Water, Mobile phase B: MeCN; Flow Rate: 15.0 mL/min]. LCMS (ES+): m/z 1094.1 [M+H]⁺.

### Step 2: 3-(carboxymethoxy)-4-chloro-5-[3-[[1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-4-piperidyl]amino]phenyl]thiophene-2-carboxylic acid (3)

3-(carboxymethoxy)-4-chloro-5-[3-[[1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-4-piperidyl]amino]phenyl]thiophene-2-carboxylic acid (3, 324.38 mg, 22.54 μmol, 80% yield) was synthesized from 2-[[2-tert-butoxycarbonyl-4-chloro-5-[3-[[1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-4-piperidyl]amino]phenyl]-3-thienyl]oxy]acetic acid (C-5) in a similar fashion to Compound C-1, except using 6 eq. TFA. The residue purified by reverse phase prep HPLC [Purification method: Column: SunFire—C18 100 mm, Mobile phase A: 0.1% TFA in Water, Mobile phase B: MeCN; Flow Rate: 15.0 mL/min]. LCMS (ES+): m/z 1037.8 [M+H]⁺. ¹H NMR (400 MHz, DMSO-d6): δ 13.20 (s, 1H), 11.12 (s, 1H), 10.01 (s, 1H), 7.78-7.73 (m, 2H), 7.71-7.67 (m, 2H), 7.57 (d, J=8.12 Hz, 1H), 7.30 (t, J=7.92 Hz, 1H), 7.21 (t, J=7.88 Hz, 1H), 7.08 (d, J=7.52 Hz, 1H), 6.88 (s, 1H), 6.82 (d, J=7.72 Hz, 1H), 6.71 (d, J=8.08 Hz, 1H), 5.90 (s, 1H), 5.15-5.10 (m, 1H), 4.92 (s, 2H), 4.36 (s, 2H), 3.70 (t, J=6.12 Hz, 2H), 3.56-3.39 (m, 13H), 3.07-2.86 (m, 5H), 2.54-2.51 (m, 2H), 2.07-1.81 (m, 5H), 1.41-1.24 (m, 2H).

### Example 4

### 3-(carboxymethoxy)-4-chloro-5-[3-[[1-[[3-[7-[[2-[[2-(2,6-dioxo-3-piperidyl)-1,3-dioxo-isoindolin-4-yl]amino]acetyl]amino]heptanoylamino]phenyl]methylsulfonyl]-4-piperidyl]amino]phenyl]thiophene-2-carboxylic acid (4)

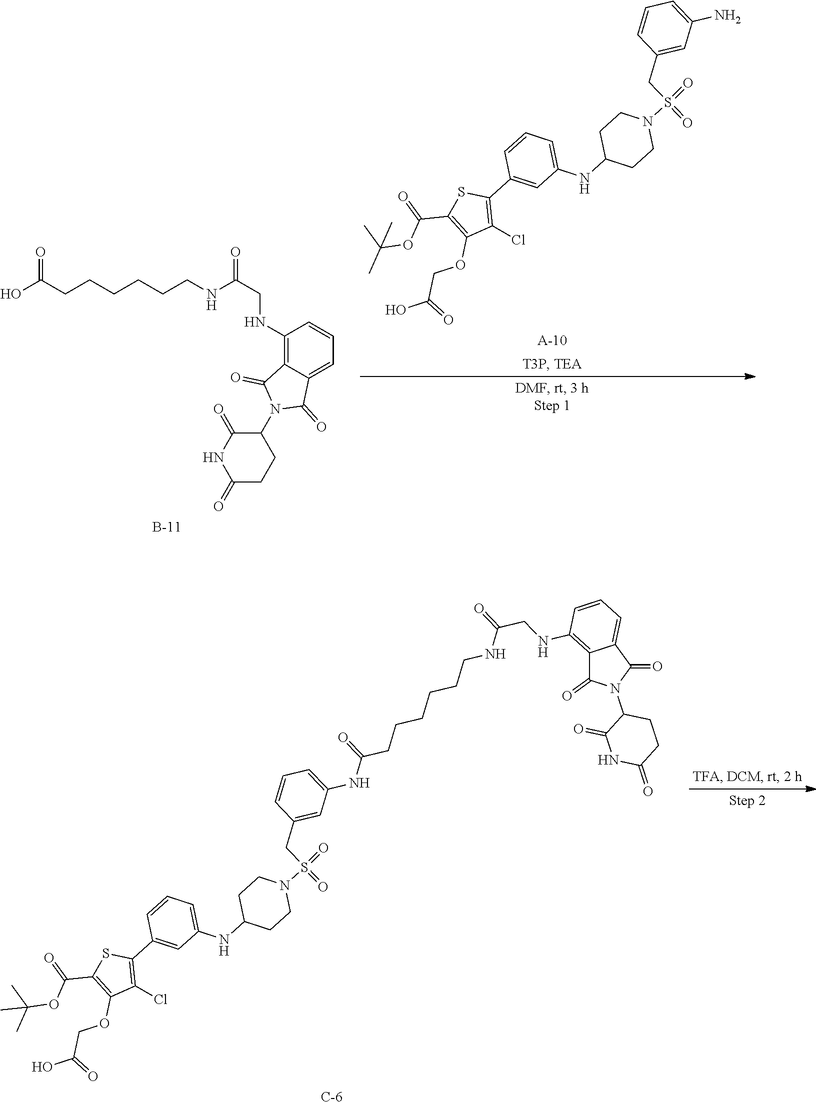

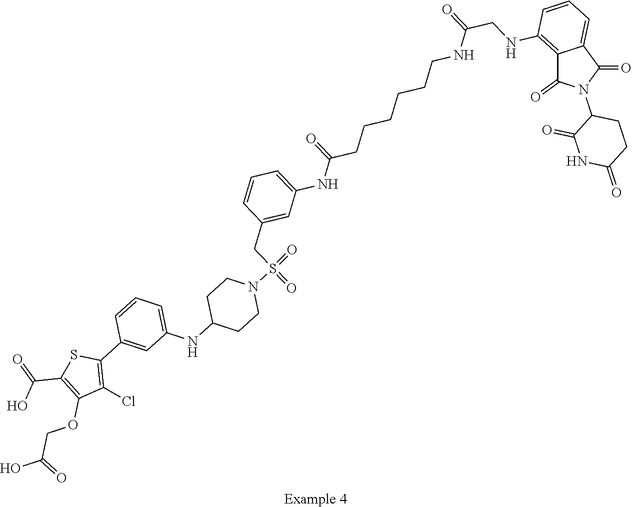

### Step 1: 2-[[2-tert-butoxycarbonyl-4-chloro-5-[3-[[1-[[3-[7-[2-[[2-(2,6-dioxo-3-piperidyl)-1,3-dioxo-isoindolin-4-yl]amino]acetyl]amino]heptanoylamino]phenyl]methylsulfonyl]-4-piperidyl]amino]phenyl]-3-thienyl]oxy]acetic acid (C-6)

Into a 50 mL round bottom flask containing a well-stirred solution of 7-[[2-[2-(2,6-dioxo-3-piperidyl)-1,3-dioxo-isoindolin-4-yl]amino]acetyl]amino]heptanoic acid (B-11, 86.48 mg, 188.63 μmol) in dry DMF (0.5 mL) were added triethylamine (57.26 mg, 565.88 μmol, 78.87 L) and 1-propanephosphonic anhydride solution (50% in Ethyl acetate) (66.44 mg, 207.49 μmol, 0.130 mL). After 10 min, 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10, 0.120 g, 188.63 μmol) was added. After 3 h, the volatiles were removed under reduced pressure and the residue diluted with water (25 mL) and extracted with ethyl acetate (2×30 mL). The organic layer was dried over sodium sulfate, filtered and the solvent removed. The residue was purified by reverse phase prep HPLC [Purification method: Column: SunFire C18 (19×150 mm) 5 μm, Mobile phase A: 0.1% TFA in water, Mobile phase B: MeCN; Flow Rate: 15.0 mL/min] to afford 2-[[2-tert-butoxycarbonyl-4-chloro-5-[3-[[1-[[3-[7-[[2-[[2-(2,6-dioxo-3-piperidyl)-1,3-dioxo-isoindolin-4-yl]amino]acetyl]amino]heptanoylamino]phenyl]methylsulfonyl]-4-piperidyl]amino]phenyl]-3-thienyl]oxy]acetic acid (C-6, 40 mg, 35.80 μmol, 19% yield, TFA salt) as a yellow solid. LCMS (ES+): m/z 1075.8 [M+H]⁺.

### Step 2: 3-(carboxymethoxy)-4-chloro-5-[3-[[1-[[3-[7-[[2-[[2-(2,6-dioxo-3-piperidyl)-1,3-dioxo-isoindolin-4-yl]amino]acetyl]amino]heptanoylamino]phenyl]methylsulfonyl]-4-piperidyl]amino]phenyl]thiophene-2-carboxylic acid (4)

3-(carboxymethoxy)-4-chloro-5-[3-[[1-[[3-[7-[[2-[[2-(2,6-dioxo-3-piperidyl)-1,3-dioxo-isoindolin-4-yl]amino]acetyl]amino]heptanoylamino]phenyl]methylsulfonyl]-4-piperidyl]amino]phenyl]thiophene-2-carboxylic acid (4, 0.020 g, 19.01 μmol, 59% yield, TFA salt) was synthesized from 2-[[2-tert-butoxycarbonyl-4-chloro-5-[3-[[1-[[3-[7-[[2-[[2-(2,6-dioxo-3-piperidyl)-1,3-dioxo-isoindolin-4-yl]amino]acetyl]amino]heptanoylamino]phenyl]methylsulfonyl]-4-piperidyl]amino]phenyl]-3-thienyl]oxy]acetic acid (C-6) in a similar fashion to Compound C-1. The residue purified by reverse phase prep HPLC [Purification method: Column: SunFire C18 (19×150 mm) 5 μm, Mobile phase A: 0.1% TFA in water, Mobile phase B: MeCN; Flow Rate: 15.0 mL/min]. LCMS (ES+): m/z 1020.2 [M+H]⁺. ¹H NMR (400 MHz, DMSO-d6): δ 13.30 (bs, 1H), 11.11 (s, 1H), 9.96 (s, 1H), 8.08 (s, 1H), 7.68 (s, 1H), 7.61-7.56 (m, 2H), 7.30 (t, J=7.88 Hz, 1H), 7.21 (t, J=7.68 Hz, 1H), 7.08-7.06 (m, 2H), 7.00-6.80 (m, 4H), 6.71 (s, 1H), 5.95-5.73 (m, 3H), 5.08 (dd, J=5.36, 12.52 Hz, 1H), 4.93 (s, 2H), 4.37 (s, 2H), 3.92 (s, 2H), 3.53-3.45 (m, 3H), 3.10-3.08 (m, 3H), 2.99-2.87 (m, 4H), 2.68-2.61 (m, 2H), 2.32-2.28 (m, 3H), 2.04-1.94 (m, 3H), 1.68-1.52 (m, 2H), 1.41-1.18 (m, 8H).

### Example 5

### 3-(carboxymethoxy)-4-chloro-5-[3-[[1-[[3-[3-[2-[2-[[2-[2-(2,6-dioxo-3-piperidyl)-1,3-dioxo-isoindolin-4-yl]oxyacetyl]amino]ethoxy]ethoxy]propanoylamino]phenyl]methylsulfonyl]-4-piperidyl]amino]phenyl]thiophene-2-carboxylic acid (5)

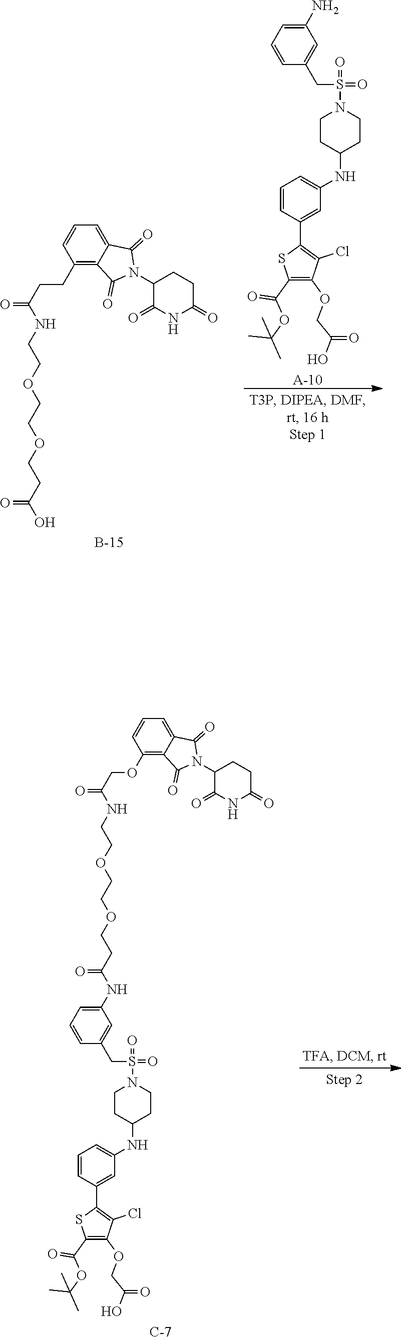

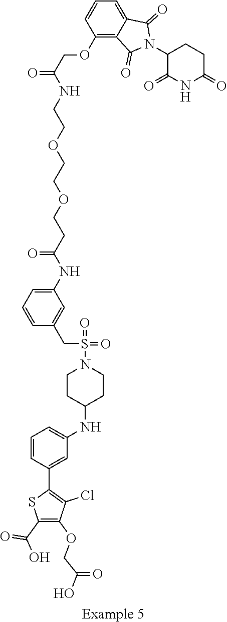

### Step 1: 2-[2-tert-butoxycarbonyl-4-chloro-5-[3-[[1-[[3-[3-[2-[2-[[2-[2-(2,6-dioxo-3-piperidyl)-1,3-dioxo-isoindolin-4-yl]oxyacetyl]amino]ethoxy]ethoxy]propanoylamino]phenyl]methylsulfonyl]-4-piperidyl]amino]phenyl]-3-thienyl]oxy]acetic acid (C-7)

2-[[2-tert-butoxycarbonyl-4-chloro-5-[3-[[1-[[3-[3-[2-[2-[[2-[2-(2,6-dioxo-3-piperidyl)-1,3-dioxo-isoindolin- 4-yl]oxyacetyl]amino]ethoxy]ethoxy]propanoylamino]phenyl]methylsulfonyl]-4-piperidyl]amino]phenyl]-3-thienyl]oxy]acetic acid (C-7, 70 mg, 25.23 μmol, 40% yield) was synthesized from 3-[2-[2-[[2-[2-(2,6-dioxo-3-piperidyl)-1,3-dioxo-isoindolin-4-yl]oxyacetyl]amino]ethoxy]ethoxy]propanoic acid (B-15) and 2-1 [5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Compound-C6 except using 4.0 eq. of DIPEA. This material was used without purification. UPLC-MS (ES+): m/z 1109.7 [M+H]⁺.

### Step 2: 3-(carboxymethoxy)-4-chloro-5-[3-[[1-[[3-[3-[2-[2-[[2-[2-(2,6-dioxo-3-piperidyl)-1,3-dioxo-isoindolin-4-yl]oxyacetyl]amino]ethoxy]ethoxy]propanoylamino]phenyl]methylsulfonyl]-4-piperidyl]amino]phenyl]thiophene-2-carboxylic acid (5)

3-(carboxymethoxy)-4-chloro-5-[3-[[1-[[3-[3-[2-[2-[[2-[2-(2,6-dioxo-3-piperidyl)-1,3-dioxo-isoindolin-4-yl]oxyacetyl]amino]ethoxy]ethoxy]propanoylamino]phenyl]methylsulfonyl]-4-piperidyl]amino]phenyl]thiophene-2-carboxylic acid (5, 6.58 mg, 5.69 μmol, 9% yield, TFA salt) was synthesized from 2-[[2-tert-butoxycarbonyl-4-chloro-5-[3-[[1-[[3-[3-[2-[2-[[2-[2-(2,6-dioxo-3-piperidyl)-1,3-dioxo-isoindolin-4-yl]oxyacetyl]amino]ethoxy]ethoxy]propanoylamino]phenyl]methylsulfonyl]-4-piperidyl]amino]phenyl]-3-thienyl]oxy]acetic acid (C-7) in a similar fashion to Compound C-1, except using 100 eq. of TFA. The residue purified by reverse phase prep HPLC [Purification method: Column: SunFire C18 (19×150 mm) 5 μm, Mobile phase: 0.1% TFA in water and MeCN]. LC-MS (ES+): m/z 1053.3 [M+H]⁺. ¹H NMR (400 MHz, DMSO-d6): δ 13.00 (bs, 1H), 11.13 (s, 1H), 10.03 (s, 1H), 8.61 (s, 1H), 8.02-8.00 (m, 1H), 7.82-7.78 (m, 1H), 7.67-7.57 (m, 2H), 7.49 (d, J=7.24 Hz, 1H), 7.38 (d, J=8.56 Hz, 1H), 7.30 (t, J=7.88 Hz, 1H), 7.21 (t, J=7.88 Hz, 1H), 7.08 (d, J=7.52 Hz, 1H), 6.88 (s, 1H), 6.82 (d, J=7.64 Hz, 1H), 6.71 (d, J=8.24 Hz, 1H), 5.92 (s, 1H), 5.14-5.10 (m, 1H), 4.92 (s, 2H), 4.78 (s, 2H), 4.36 (s, 2H), 3.77-3.62 (m, 3H), 3.43-3.27 (m, 6H), 3.17-2.86 (m, 3H), 2.68-2.62 (m, 3H), 2.33-1.94 (m, 6H), 1.41-1.24 (m, 3H).

### Example 6

### 3-(carboxymethoxy)-4-chloro-5-(3-((1-((3-(7-(2-((2-(2,6-dioxopiperidin-3-yl)-1,3-dioxoisoindolin-4-yl)(methyl)amino)acetamido)heptanamido)benzyl)sulfonyl)piperidin-4-yl)amino)phenyl)thiophene-2-carboxylic acid (6)

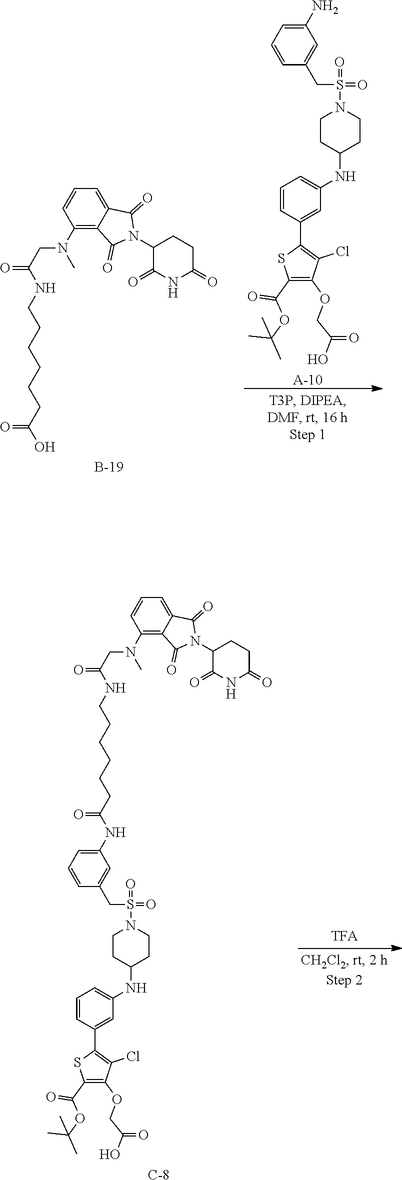

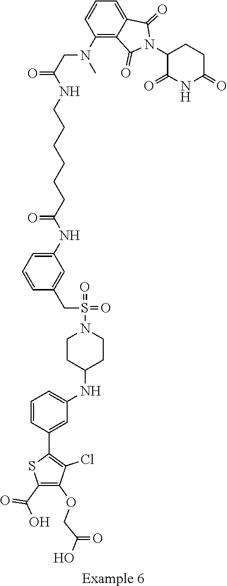

### Step 1: 2-((2-(tert-butoxycarbonyl)-4-chloro-5-(3-((1-((3-(7-(2-((2-(2,6-dioxopiperidin-3-yl)-1,3-dioxoisoindolin-4-yl)(methyl)amino)acetamido)heptanamido)benzyl)sulfonyl)piperidin-4-yl)amino)phenyl)thiophen-3-yl)oxy)acetic acid (C-8)

2-((2-(tert-butoxycarbonyl)-4-chloro-5-(3-((1-((3-(7-(2-((2-(2,6-dioxopiperidin-3-yl)-1,3-dioxoisoindolin-4-yl)(methyl)amino)acetamido)heptanamido)benzyl)sulfonyl)piperidin-4-yl)amino)phenyl)thiophen-3-yl)oxy)acetic acid (C-8, 12 mg, 10.23 μmol, 16% yield) was synthesized from 7-[[2-[[2-(2,6-dioxo-3-piperidyl)-1,3-dioxo-isoindolin-4-yl]-methyl-amino]acetyl]amino]heptanoic acid (B-19) and 2-[5-[3-[[1-[(3-aminophenyl)methylsulfonyl]-4-piperidyl]amino]phenyl]-2-tert-butoxycarbonyl-4-chloro-3-thienyl]oxy]acetic acid (A-10), in a similar fashion to Compound-C6, except using 4.0 eq. of DIPEA. The residue was purified by reverse phase preparative HPLC [Column: SunFire C18 (19×150 mm) 5 μm, Mobile phase: 0.1% TFA in water and MeCN]. LCMS (ES+): m/z 1090.3 [M+H]⁺.

### Step 3-(carboxymethoxy)-4-chloro-5-(3-((1-((3-(7-(2-((2-(2,6-dioxopiperidin-3-yl)-1,3-dioxoisoindolin-4-yl)(methyl)amino)acetamido)heptanamido)benzyl)sulfonyl)piperidin-4-yl)amino)phenyl)thiophene-2-carboxylic acid (6)

3-(carboxymethoxy)-4-chloro-5-(3-((1-((3-(7-(2-((2-(2,6-dioxopiperidin-3-yl)-1,3-dioxoisoindolin-4-yl)(methyl)amino)acetamido)heptanamido)benzyl)sulfonyl)piperidin-4-yl)amino)phenyl)thiophene-2-carboxylic acid (6, 5.77 mg, 5.41 μmol, 49% yield, TFA salt) was synthesized from 2-((2-(tert-butoxycarbonyl)-4-chloro-5-(3-((1-((3-(7-(2-((2-(2,6-dioxopiperidin-3-yl)-1,3-dioxoisoindolin-4-yl)(methyl)amino)acetamido)heptanamido)benzyl)sulfonyl)piperidin-4-yl)amino)phenyl)thiophen-3-yl)oxy)acetic acid (C-8) in a similar fashion to Compound C-1 except using 480 eq. of TFA. LCMS (ES+): m/z 1034.6 [M+H]⁺. ¹H NMR (400 MHz, DMSO-d6) δ 11.07 (s, 1H), 9.94 (s, 1H), 7.89 (t, J=5.7 Hz, 1H), 7.67 (s, 1H), 7.65-7.59 (m, 1H), 7.56 (d, J=8.1 Hz, 1H), 7.29 (t, J=7.8 Hz, 1H), 7.22 (dt, J=15.3, 7.4 Hz, 3H), 7.07 (d, J=6.9 Hz, 1H), 6.86 (s, 1H), 6.81 (d, J=7.6 Hz, 1H), 6.72-6.66 (m, 1H), 5.90-5.82 (m, 1H), 5.08 (dd, J=12.9, 5.4 Hz, 1H), 4.86 (s, 2H), 4.36 (s, 2H), 4.22-4.06 (m, 2H), 3.59-3.49 (m, 3H), 3.09-3.02 (m, 2H), 2.99 (s, 3H), 2.97-2.81 (m, 3H), 2.35-2.25 (m, 3H), 2.05-1.89 (m, 4H), 1.64-1.51 (m, 2H), 1.44-1.32 (m, 4H), 1.31-1.20 (m, 6H).

### Example 7

### 3-(carboxymethoxy)-4-chloro-5-[3-[[1-[[3-[7-[[2-[[2-(2,6-dioxo-3-piperidyl)-1-oxo-isoindolin-4-yl]amino]acetyl]amino]heptanoylamino]phenyl]methylsulfonyl]-4-piperidyl]amino]phenyl]thiophene-2-carboxylic acid (7)

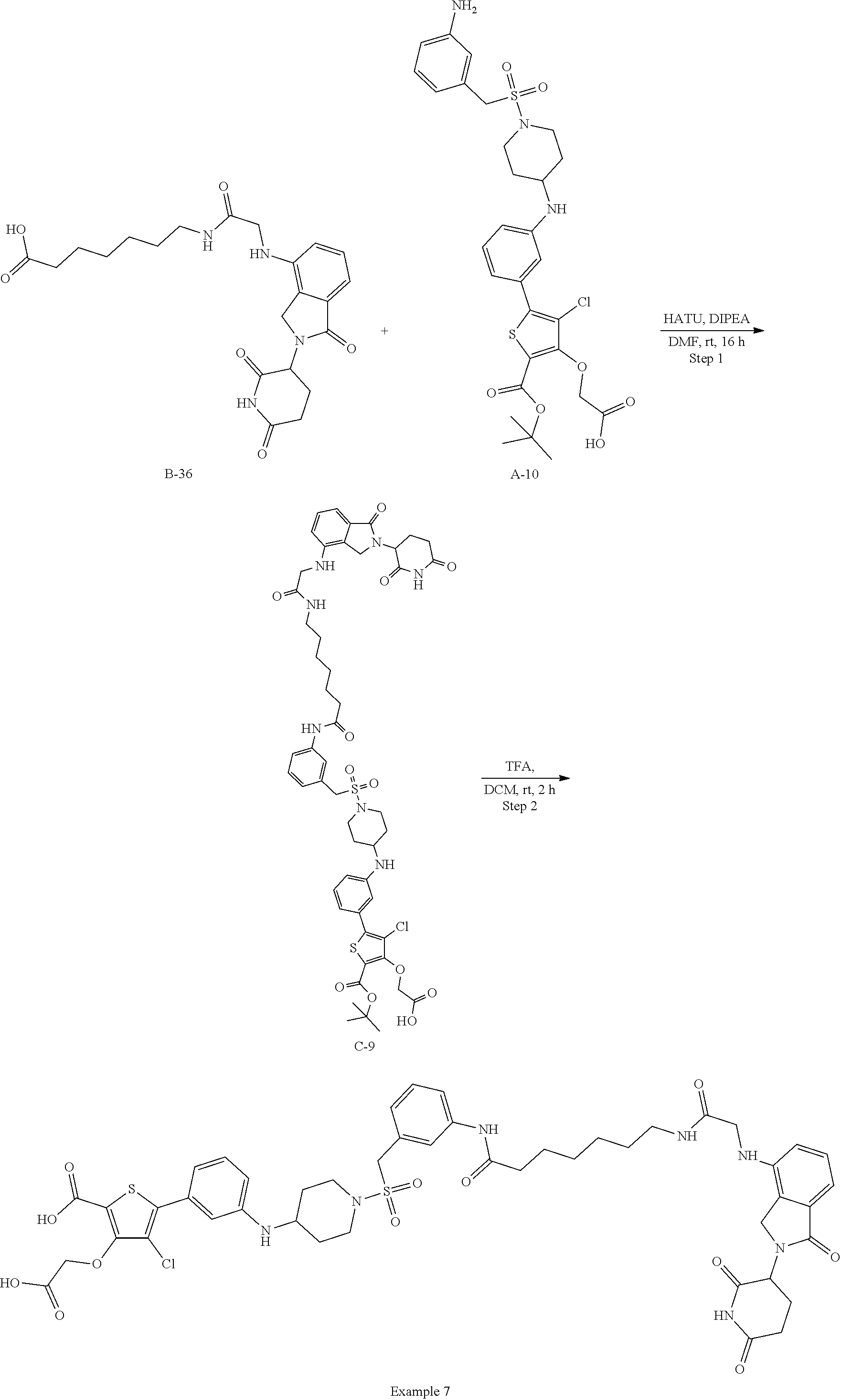

### Step 1: 2-[[2-tert-butoxycarbonyl-4-chloro-5-[3-[[1-[[3-[7-[[2-[[2-(2,6-dioxo-3-piperidyl)-1-oxo-isoindolin-4-yl]amino]acetyl]amino]heptanoylamino]phenyl]methylsulfonyl]-4-piperidyl]amino]phenyl]-3-thienyl]oxy]acetic acid (C-9)

2-[[2-tert-butoxycarbonyl-4-chloro-5-[3-[[1-[[3-[7-[[2-[[2-(2,6-dioxo-3-piperidyl)-1-oxo-isoindolin-4-yl]amino]acetyl]amino]heptanoylamino]phenyl]methylsulfonyl]-4-piperidyl]amino]phenyl]-3-thienyl]oxy]acetic acid (C-9, 35 mg, 24.82 μmol, 21% yield, TFA salt) was synthesized from 7-[[2-[[2-(2,6-dioxo-3-piperidyl)-1-oxo-isoindolin-4-yl]amino]acetyl]amino]heptanoic acid (B-36) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1 except using DMF as the solvent. The residue was purified by reverse phase preparative HPLC [Purification method: Column: SunFire C18 (19×150 mm) 5 μm; Mobile phase A-0.1% TFA in water and Mobile phase B-MeCN]. LCMS (ES+): m/z 1062.2 [M+H]⁺.

### Step 2: 3-(carboxymethoxy)-4-chloro-5-[3-[[1-[[3-[7-[[2-[[2-(2,6-dioxo-3-piperidyl)-1-oxo-isoindolin-4-yl]amino]acetyl]amino]heptanoylamino]phenyl]methylsulfonyl]-4-piperidyl]amino]phenyl]thiophene-2-carboxylic acid (7)

3-(carboxymethoxy)-4-chloro-5-[3-[[1-[[3-[7-[[2-[[2-(2,6-dioxo-3-piperidyl)-1-oxo-isoindolin-4-yl]amino]acetyl]amino]heptanoylamino]phenyl]methylsulfonyl]-4-piperidyl]amino]phenyl]thiophene-2-carboxylic acid (7, 15.22 mg, 13.48 μmol, 45% yield) was synthesized from 2-[[2-tert-butoxycarbonyl-4-chloro-5-[3-[[1-[[3-[7-[[2-[[2-(2,6-dioxo-3-piperidyl)-1-oxo-isoindolin-4-yl]amino]acetyl]amino]heptanoylamino]phenyl]methylsulfonyl]-4-piperidyl]amino]phenyl]-3-thienyl]oxy]acetic acid (C-9) in a similar fashion to Compound C-1, except using 15 eq. of TFA. The residue was purified by reverse phase preparative HPLC [Purification method: Column: SunFire C18 (19×150 mm) 5 μm; Mobile phase A-0.1% TFA in water and Mobile phase B-MeCN]. LCMS (ES+): m/z 1006.1 [M+H]⁺. ¹H NMR (400 MHz, DMSO-d6): 11.02 (s, 1H), 9.95 (s, 1H), 7.96-7.93 (m, 1H), 7.67 (s, 1H), 7.57 (d, J=8.40 Hz, 1H), 7.32-7.26 (m, 2H), 7.23-7.19 (m, 1H), 7.07 (d, J=7.68 Hz, 1H), 6.98 (d, J=7.44 Hz, 1H), 6.88 (s, 1H), 6.82 (d, J=7.44 Hz, 1H), 6.71 (d, J=8.32 Hz, 1H), 6.55 (d, J=8.04 Hz, 1H), 5.90 (bs, 1H), 5.13 (dd, J=5.04, 13.22 Hz, 1H), 4.92 (s, 2H), 4.36 (s, 2H), 4.31-4.17 (m, 3H), 3.72 (s, 2H), 3.56-3.53 (m, 3H), 3.08-2.89 (m, 6H), 2.65-2.61 (m, 2H), 2.30-2.27 (m, 3H), 2.04-1.91 (m, 4H), 1.58-1.52 (m, 2H), 1.41-1.31 (m, 4H), 1.28-1.19 (m, 4H).

### Example 8

### 3-(carboxymethoxy)-4-chloro-5-[3-[[1-[[3-[3-[2-[2-[[2-[2-(2,6-dioxo-3-piperidyl)-1,3-dioxo-isoindolin-4-yl]oxyethoxy]ethoxy]ethoxy]propanoylamino]phenyl]methylsulfonyl]-4-piperidyl]amino]phenyl]thiophene-2-carboxylic acid (8)

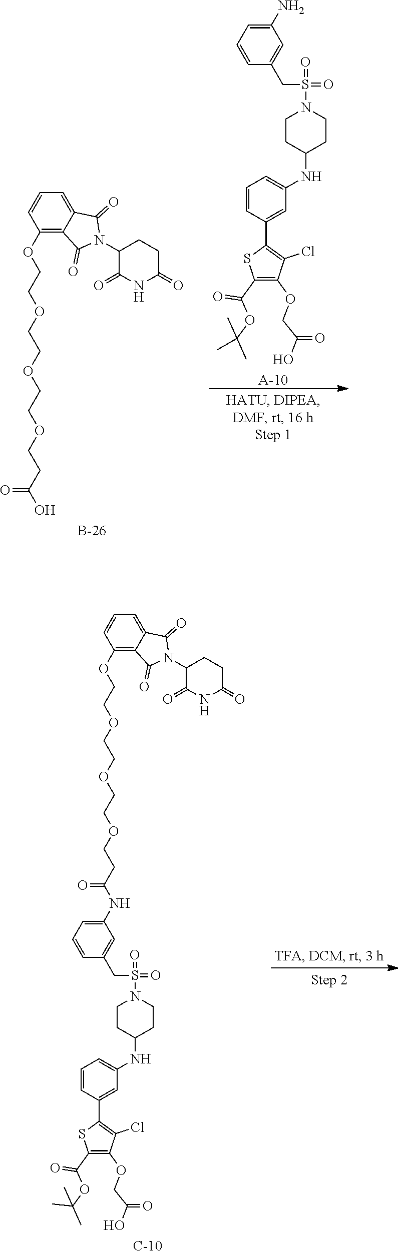

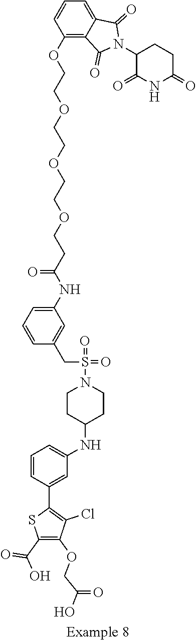

### Step 1: 2-[[2-tert-butoxycarbonyl-4-chloro-5-[3-[[1-[[3-[3-[2-[2-[[2-[2-(2,6-dioxo-3-piperidyl)-1,3-dioxo-isoindolin-4-yl]oxyethoxy]ethoxy]ethoxy]propanoylamino]phenyl]methylsulfonyl]-4-piperidyl]amino]phenyl]-3-thienyl]oxy]acetic acid (C-10)

2-[[2-tert-butoxycarbonyl-4-chloro-5-[3-[[1-[[3-[3-[2-[2-[[2-[2-(2,6-dioxo-3-piperidyl)-1,3-dioxo-isoindolin- 4-yl]oxyethoxy]ethoxy]ethoxy]propanoylamino]phenyl]methylsulfonyl]-4-piperidyl]amino]phenyl]-3-thienyl]oxy]acetic acid (C-10, 100 mg, 67.48 μmol, 72% yield) was synthesized from 3-[2-[2-[[2-[2-(2,6-dioxo-3-piperidyl)-1,3-dioxo-isoindolin-4-yl]oxyethoxy]ethoxy]ethoxy]propanoic acid (B-26) and 2-[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. UPLC-MS (ES+): m/z 1096.7 [M+H]⁺.

### Step 2: 3-(carboxymethoxy)-4-chloro-5-[3-[[1-[[3-[3-[2-[2-[[2-[2-(2,6-dioxo-3-piperidyl)-1,3-dioxo-isoindolin-4-yl]oxyethoxy]ethoxy]ethoxy]propanoylamino]phenyl]methylsulfonyl]-4-piperidyl]amino]phenyl]thiophene-2-carboxylic acid (8)

3-(carboxymethoxy)-4-chloro-5-[3-[[1-[[3-[3-[2-[2-[[2-[2-(2,6-dioxo-3-piperidyl)-1,3-dioxo-isoindolin-4-yl]oxyethoxy]ethoxy]ethoxy]propanoylamino]phenyl]methylsulfonyl]-4-piperidyl]amino]phenyl]thiophene-2-carboxylic acid (8, 5.9 mg, 5.67 μmol, 6.2% yield) was synthesized from 2-[[2-tert-butoxycarbonyl-4-chloro-5-[3-[[1-[[3-[3-[2-[2-[[2-[2-(2,6-dioxo-3-piperidyl)-1,3-dioxo-isoindolin-4-yl]oxyethoxy]ethoxy]ethoxy]propanoylamino]phenyl]methylsulfonyl]-4-piperidyl]amino]phenyl]-3-thienyl]oxy]acetic acid (C-10) in a similar fashion to Compound C-1, except using 30 eq. of TFA. The residue was purified by reverse phase prep HPLC [Purification method: Column: SunFire C18 (19×150 mm), 5 μm; Mobile phase A: 0.1% TFA in water and Mobile phase B: MeCN]. LCMS (ES+): m/z 1041.3 [M+H]⁺. ¹H NMR (400 MHz, DMSO-d6): δ 11.11 (s, 1H), 10.02 (d, J=Hz, 1H), 7.80 (t, J=7.84 Hz, 1H), 7.68 (s, 1H), 7.58 (d, J=8.48 Hz, 1H), 7.51 (d, J=8.56 Hz, 1H), 7.45 (s, 1H), 7.30 (t, J=7.80 Hz, 1H), 7.23-7.19 (m, 2H), 7.08-7.07 (m, 2H), 6.95 (s, 1H), 6.88 (s, 1H), 6.82 (d, J=7.44 Hz, 1H), 6.71 (d, J=8.12 Hz, 1H), 5.11-5.06 (m, 1H), 4.93 (s, 2H), 0.36 (s, 2H), 4.32 (t, J=4.52 Hz, 2H), 3.79-3.50 (m, 14H), 2.99-2.68 (m, 5H), 1.97-1.94 (m, 4H), 1.38-1.24 (m, 4H).

### Example 9

### 3-(carboxymethoxy)-4-chloro-5-[3-[[1-[[3-[3-[2-[2-[[2-[2-(2,6-dioxo-3-piperidyl)-1-oxo-isoindolin-4-yl]oxyacetyl]amino]ethoxy]ethoxy]propanoylamino]phenyl]methylsulfonyl]-4-piperidyl]amino]phenyl]thiophene-2-carboxylic acid (9)

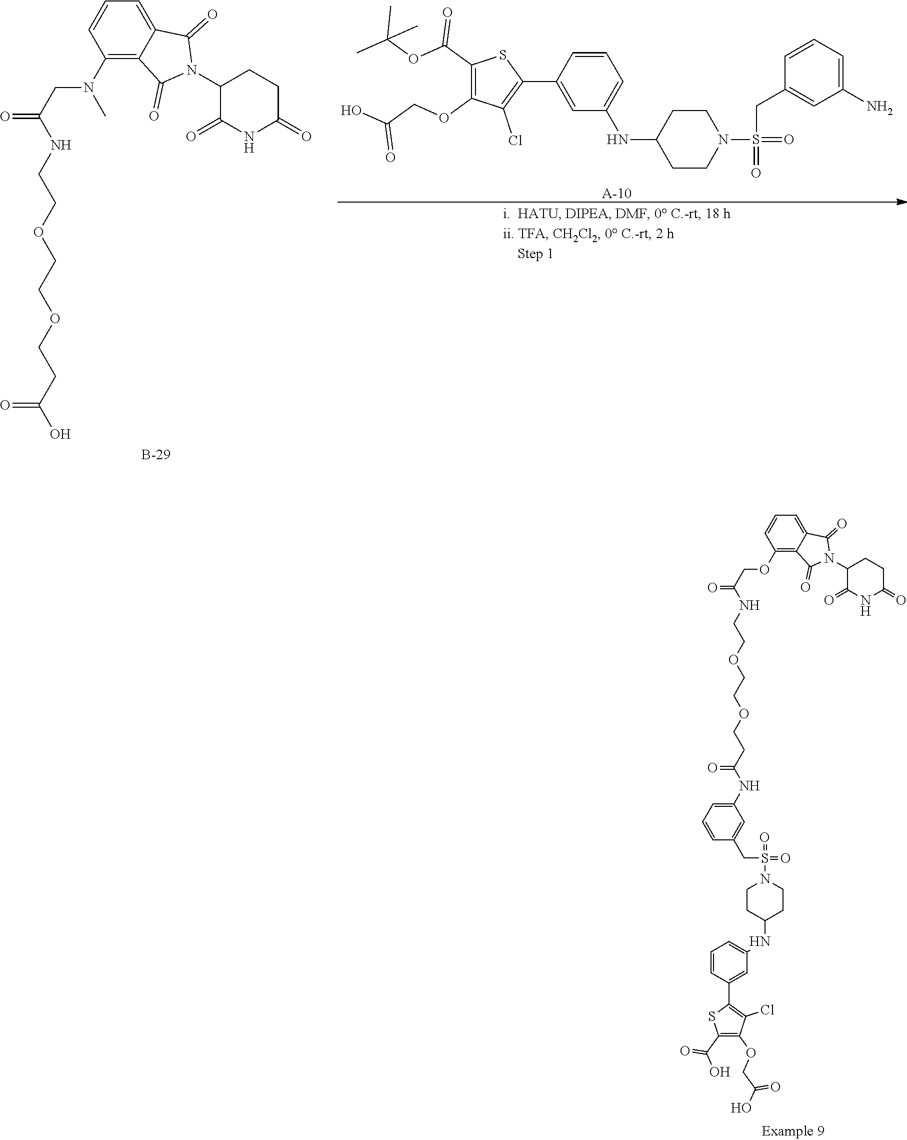

### Step 1: 3-(carboxymethoxy)-4-chloro-5-[3-[[1-[[3-[3-[2-[2-[[2-[2-(2,6-dioxo-3-piperidyl)-1-oxo-isoindolin-4-yl]oxyacetyl]amino]ethoxy]ethoxy]propanoylamino]phenyl]methylsulfonyl]-4-piperidyl]amino]phenyl]thiophene-2-carboxylic acid (9)

3-(carboxymethoxy)-4-chloro-5-[3-[[1-[[3-[3-[2-[2-[[2-[2-(2,6-dioxo-3-piperidyl)-1-oxo-isoindolin-4-yl]oxyacetyl]amino]ethoxy]ethoxy]propanoylamino]phenyl]methylsulfonyl]-4-piperidyl]amino]phenyl]thiophene-2-carboxylic acid (9, 4.56 mg, 4.21 μmol, 5% yield, TFA salt) was synthesized from 3-[2-[2-[[2-[2-(2,6-dioxo-3-piperidyl)-1-oxo-isoindolin-4-yl]oxyacetyl]amino]ethoxy]ethoxy]propanoic acid (B-29) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The crude residue was then treated with TFA in a similar fashion to Compound C-1, except 4 eq. of TFA was used. The crude was purified by reverse-phase preparative HPLC [Purification method: Column: SunFire C18 (19×150 mm) 5 μm, Mobile phase: 0.1% TFA in water and MeCN]. LCMS (ES+): m/z 1039.2 [M+H]⁺. ¹H NMR (400 MHz, DMSO-d6): δ 11.02 (s, 1H), 10.03 (s, 1H), 8.13 (s, 1H), 7.67 (t, J=Hz, 1H), 7.59-7.58 (m, 1H), 7.49-7.45 (m, 1H), 7.36-7.29 (m, 2H), 7.24-7.20 (m, 1H), 7.14-7.08 (m, 2H), 6.89 (s, 1H), 6.83 (d, J=7.08 Hz, 1H), 6.72 (d, J=8.04 Hz, 1H), 5.16-5.12 (m, 1H), 5.02 (s, 2H), 4.64 (s, 2H), 4.49-4.30 (m, 4H), 3.70-3.68 (m, 2H), 3.50-3.42 (m, 11H), 3.30-3.22 (m, 2H), 3.12-2.85 (m, 4H), 2.01-1.94 (m, 4H), 1.48-1.47 (m, 2H).

### Example 10

### 3-(carboxymethoxy)-4-chloro-5-[3-[[1-[[3-[7-[[2-[2-(2,6-dioxo-3-piperidyl)-1-oxo-isoindolin-4-yl]oxyacetyl]amino]heptanoylamino]phenyl]methylsulfonyl]-4-piperidyl]amino]phenyl]thiophene-2-carboxylic acid (10)

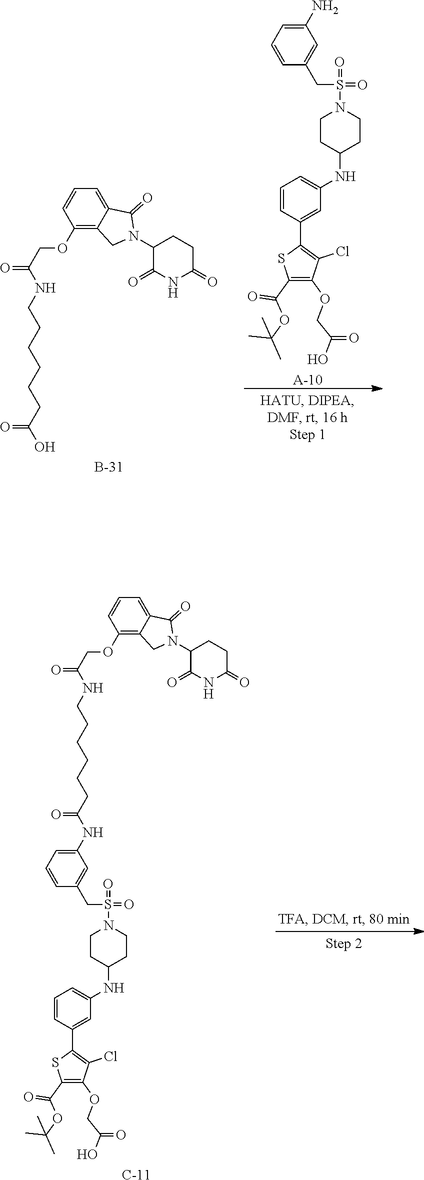

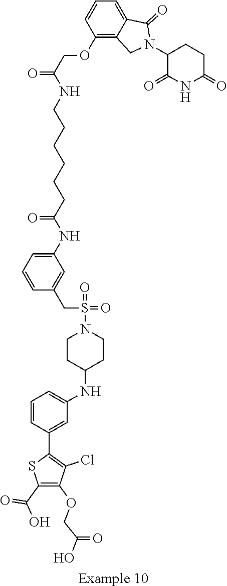

### Step 1: 2-[[2-tert-butoxycarbonyl-4-chloro-5-[3-[[1-[[3-[7-[[2-[2-(2,6-dioxo-3-piperidyl)-1-oxo-isoindolin-4-yl]oxyacetyl]amino]heptanoylamino]phenyl]methylsulfonyl]-4-piperidyl]amino]phenyl]-3-thienyl]oxy]acetic acid (C-11)

2-[[2-tert-butoxycarbonyl-4-chloro-5-[3-[[1-[[3-[7-[2-[2-(2,6-dioxo-3-piperidyl)-1-oxo-isoindolin-4-yl]oxyacetyl]amino]heptanoylamino]phenyl]methylsulfonyl]-4-piperidyl]amino]phenyl]-3-thienyl]oxy]acetic acid (C-11, 35 mg, 31.59 μmol, 40% yield) was synthesized from 7-[[2-[2-(2,6-dioxo-3-piperidyl)-1-oxo-isoindolin-4-yl]oxyacetyl]amino]heptanoic acid (B-31) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as a solvent. The crude product was purified by reverse phase prep HPLC [Purification method: Column: SunFire C18 (19×150 mm) 5 μm, Mobile phase: 0.1% TFA in water and MeCN]. LCMS (ES+): m/z 1063.3 [M+H]⁺.

### Step 2: 3-(carboxymethoxy)-4-chloro-5-[3-[[1-[[3-[7-[[2-[2-(2,6-dioxo-3-piperidyl)-1-oxo-isoindolin-4-yl]oxyacetyl]amino]heptanoylamino]phenyl]methylsulfonyl]-4-piperidyl]amino]phenyl]thiophene-2-carboxylic acid (10)

3-(carboxymethoxy)-4-chloro-5-[3-[[1-[[3-[7-[2-[2-(2,6-dioxo-3-piperidyl)-1-oxo-isoindolin-4-yl]oxyacetyl]amino]heptanoylamino]phenyl]methylsulfonyl]-4-piperidyl]amino]phenyl]thiophene-2-carboxylic acid (10, 17.25 mg, 16.78 μmol, 51% yield, TFA salt) was synthesized from 2-[[2-tert-butoxycarbonyl-4-chloro-5-[3-[[1-[[3-[7-[[2-[2-(2,6-dioxo-3-piperidyl)-1-oxo-isoindolin-4-yl]oxyacetyl]amino]heptanoylamino]phenyl]methylsulfonyl]-4-piperidyl]amino]phenyl]-3-thienyl]oxy]acetic acid (C-11) in a similar fashion to Compound C-1, except using 130 eq. of TFA. The crude product was purified by reverse phase prep HPLC [Purification method: Column: SunFire C18 (19×150 mm) 5 μm, Mobile phase: 0.1% TFA in water and MeCN]. LCMS (ES+): m/z 1007.1 [M+H]⁺. ¹H NMR (400 MHz, DMSO-d₆) δ 11.00 (s, 1H), 9.94 (s, 1H), 8.10-8.03 (m, 1H), 7.67 (s, 1H), 7.56 (d, J=8.3 Hz, 1H), 7.46 (t, J=7.8 Hz, 1H), 7.37-7.26 (m, 2H), 7.20 (t, J=7.9 Hz, 1H), 7.11 (d, J=8.1 Hz, 1H), 7.07 (d, J=7.6 Hz, 1H), 6.87 (s, 1H), 6.81 (d, J=7.6 Hz, 1H), 6.69 (d, J=8.3 Hz, 1H), 5.92-5.81 (m, 1H), 5.13 (dd, J=13.2, 5.1 Hz, 1H), 4.88 (s, 2H), 4.62 (s, 2H), 4.45 (d, J=17.6 Hz, 1H), 4.40-4.30 (m, 3H), 3.58-3.50 (m, 2H), 3.16-3.07 (m, 2H), 3.01-2.86 (m, 3H), 2.63-2.57 (m, 1H), 2.35-2.25 (m, 3H), 2.06-1.89 (m, 3H), 1.61-1.51 (m, 2H), 1.48-1.34 (m, 4H), 1.31-1.18 (m, 6H).

### Example 11

### 3-(carboxymethoxy)-4-chloro-5-[3-[[1-[[3-[2-[2-(2,6-dioxo-3-piperidyl)-1-oxo-isoindolin-4-yl]oxyacetyl]amino]phenyl]methylsulfonyl]-4-piperidyl]amino]phenyl]thiophene-2-carboxylic acid (11)

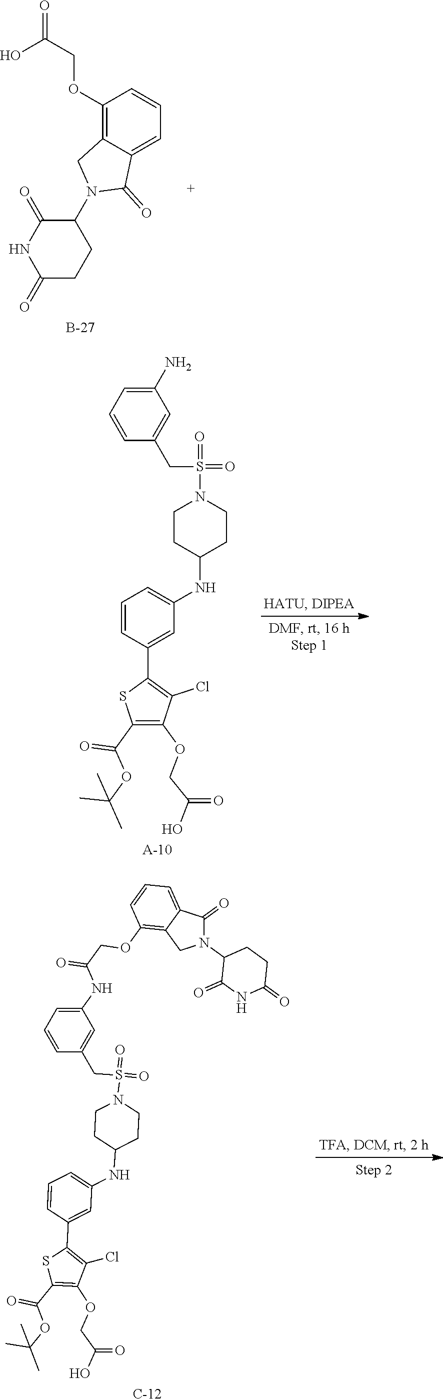

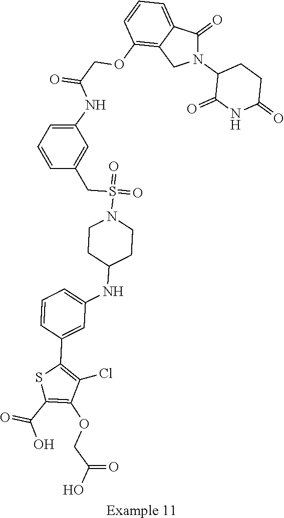 [SMILES: O=C(O)COc1c(C(=O)O)sc(-c2cccc(NC3CCN(S(=O)(=O)Cc4cccc(NC(=O)COc5cccc6c5CN(C5CCC(=O)NC5=O)C6=O)c4)CC3)c2)c1Cl ; "Example 11"]

### Step 1: 2-[[2-tert-butoxycarbonyl-4-chloro-5-[3-[[1-[[3-[[2-[2-(2,6-dioxo-3-piperidyl)-1-oxo-isoindolin-4-yl]oxyacetyl]amino]phenyl]methylsulfonyl]-4-piperidyl]amino]phenyl]-3-thienyl]oxy]acetic acid (C-12)

2-[[2-tert-butoxycarbonyl-4-chloro-5-[3-[[1-[[3-[[2-[2-(2,6-dioxo-3-piperidyl)-1-oxo-isoindolin-4-yl]oxyacetyl]amino]phenyl]methylsulfonyl]-4-piperidyl]amino]phenyl]-3-thienyl]oxy]acetic acid (C-12, 9.8 mg, 8.69 μmol, 9% yield) was synthesized from 2-[2-(2,6-dioxo-3-piperidyl)-1-oxo-isoindolin-4-yl]oxyacetic acid (B-27) and 2-[5-[3-[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The crude was purified by reverse phase prep HPLC [Purification method: Column: SunFire C18 (19×150 mm) 5 μm, Mobile phase: 0.1% TFA in water and MeCN]. UPLC-MS (ES+): m/z 936.5 [M+H]⁺

### Step 2: 3-(carboxymethoxy)-4-chloro-5-[3-[[1-[[3-[[2-[2-(2,6-dioxo-3-piperidyl)-1-oxo-isoindolin-4-yl]oxyacetyl]amino]phenyl]methylsulfonyl]-4-piperidyl]amino]phenyl]thiophene-2-carboxylic acid (11)

3-(carboxymethoxy)-4-chloro-5-[3-[[1-[[3-[[2-[2-(2,6-dioxo-3-piperidyl)-1-oxo-isoindolin-4-yl]oxyacetyl]amino]phenyl]methylsulfonyl]-4-piperidyl]amino]phenyl]thiophene-2-carboxylic acid (11, 5.41 mg, 6.06 μmol, 58% yield, TFA salt) was synthesized from 2-[[2-tert-butoxycarbonyl-4-chloro-5-[3-[1-[3-[[2-[2-(2,6-dioxo-3-piperidyl)-1-oxo-isoindolin-4-yl]oxyacetyl]amino]phenyl]methylsulfonyl]-4-piperidyl]amino]phenyl]-3-thienyl]oxy]acetic acid (C-12) in a similar fashion to Compound C-1, except using 3 eq. of TFA. The crude was purified by reverse phase prep HPLC [Purification method: Column: SunFire C18 (19×150 mm) 5 μm, Mobile phase: 0.1% TFA in water and MeCN]. UPLC-MS (ES+): m/z 880.5 [M+H]⁺. ¹H NMR (400 MHz, DMSO-d6) δ 10.99 (s, 1H), 10.24 (s, 1H), 7.72-7.69 (m, 1H), 7.62-7.57 (m, 1H), 7.48 (t, J=7.8 Hz, 1H), 7.39-7.31 (m, 2H), 7.23-7.12 (m, 3H), 6.89-6.86 (m, 1H), 6.81 (d, J=7.6 Hz, 1H), 6.73-6.66 (m, 1H), 5.88 (d, J=8.0 Hz, 1H), 5.12 (dd, J=13.3, 5.1 Hz, 1H), 4.91-4.82 (m, 3H), 4.47 (d, J=17.5 Hz, 1H), 4.42-4.31 (m, 3H), 3.58-3.51 (m, 2H), 3.01-2.87 (m, 4H), 2.64-2.58 (m, 1H), 2.18 (t, J=8.1 Hz, 1H), 2.04-1.86 (m, 4H), 1.43-1.31 (m, 3H).

### Example 12

3-(carboxymethoxy)-4-chloro-5-(3-(((4S)-1-((3-(6-(4-(((2-(2,6-dioxopiperidin-3-yl)-1,3-dioxoisoindolin-4-yl)amino)methyl)-1H-1,2,3-triazol-1-yl) hexanamido)benzyl)sulfonyl)-2,2-dimethylpiperidin-4-yl)amino)phenyl)thiophene-2-carboxylic acid (12)

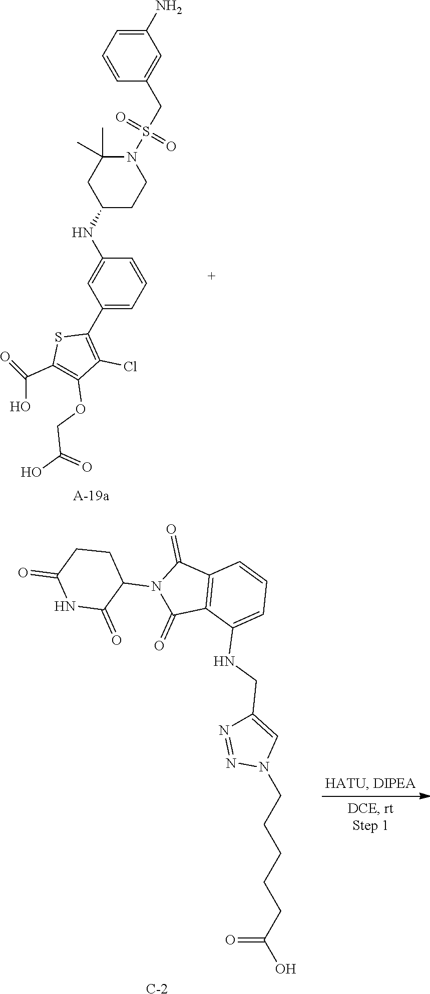

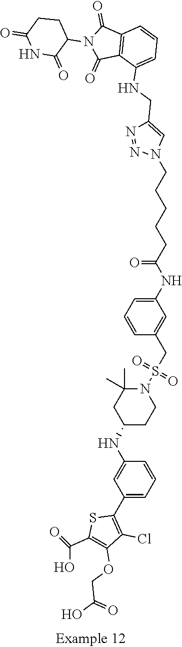 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCCCCn2cc(CNc3cccc4c3C(=O)N(C3CCC(=O)NC3=O)C4=O)nn2)c1]

### Step 1: 3-(carboxymethoxy)-4-chloro-5-(3-(((4S)-1-((3-(6-(4-(((2-(2,6-dioxopiperidin-3-yl)-1,3-dioxoisoindolin-4-yl)amino)methyl)-1H-1,2,3-triazol-1-yl) hexanamido)benzyl)sulfonyl)-2,2-dimethylpiperidin-4-yl)amino)phenyl)thiophene-2-carboxylic acid (12)

3-(carboxymethoxy)-4-chloro-5-(3-(((4S)-1-((3-(6-(4-(((2-(2,6-dioxopiperidin-3-yl)-1,3-dioxoisoindolin-4-yl)amino)methyl)-1H-1,2,3-triazol-1-yl) hexanamido)benzyl)sulfonyl)-2,2-dimethylpiperidin-4-yl)amino)phenyl)thiophene-2-carboxylic acid (12, 21.21 mg, 19.58 μmol, 48% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 6-[4-[[2-(2,6-dioxo-3-piperidyl)-1,3-dioxo-isoindolin-4-yl]amino]methyl]triazol-1-yl]hexanoic acid (C-2) in a similar fashion to Example 1. The residue was purified by reverse phase chromatography, eluting with 5-100% MeCN in water (with 0.1% TFA modifier). LCMS (ES+): 1058.465 [M+H]⁺. ¹H NMR (400 MHz, DMSO-d₆) δ 11.08 (s, 1H), 9.91 (s, 1H), 8.00 (s, 1H), 7.67-7.64 (m, 1H), 7.60-7.54 (m, 2H), 7.29 (t, J=7.9 Hz, 1H), 7.19 (t, J=7.9 Hz, 1H), 7.15 (d, J=8.6 Hz, 1H), 7.09-7.02 (m, 3H), 6.89-6.86 (m, 1H), 6.83-6.79 (m, 1H), 6.72-6.66 (m, 1H), 5.05 (dd, J=12.9, 5.4 Hz, 1H), 4.91 (s, 2H), 4.57 (s, 2H), 4.41-4.32 (m, 2H), 4.31-4.24 (m, 3H), 3.57-3.48 (m, 1H), 3.47-3.39 (m, 1H), 3.18-3.06 (m, 1H), 2.95-2.80 (m, 1H), 2.63-2.53 (m, 1H), 2.28 (t, J=7.4 Hz, 2H), 2.06-1.97 (m, 1H), 1.92-1.83 (m, 1H), 1.83-1.75 (m, 2H), 1.65-1.55 (m, 2H), 1.46 (s, 3H), 1.38 (s, 3H), 1.30-1.20 (m, 3H), 1.17-1.05 (m, 1H).

### Example 13

### 3-(carboxymethoxy)-4-chloro-5-(3-(((4S)-1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)-2,2-dimethylpiperidin-4-yl)amino)phenyl)thiophene-2-carboxylic acid (13)

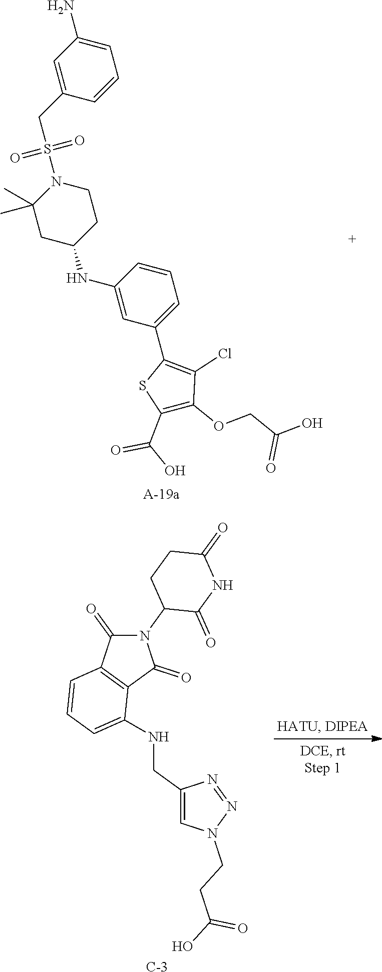

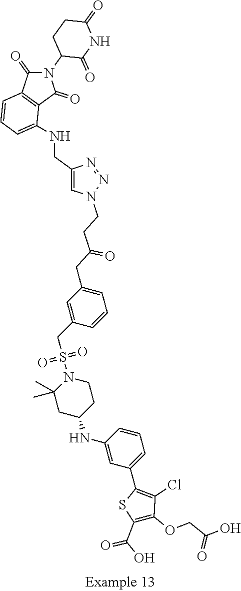 [SMILES: CC1(C)C[C@@H](Nc2cccc(-c3sc(C(=O)O)c(OCC(=O)O)c3Cl)c2)CCN1S(=O)(=O)Cc1cccc(CC(=O)CCn2cc(CNc3cccc4c3C(=O)N(C3CCC(=O)NC3=O)C4=O)nn2)c1]

### Step 3-(carboxymethoxy)-4-chloro-5-(3-(((4S)-1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)-2,2-dimethylpiperidin-4-yl)amino)phenyl)thiophene-2-carboxylic acid (13)

3-(carboxymethoxy)-4-chloro-5-(3-(((4S)-1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)-2,2-dimethylpiperidin-4-yl)amino)phenyl)thiophene-2-carboxylic acid (13, 7.3 mg, 7.18 μmol, 17% yield) was synthesized from 5-[3-[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[4-[[[2-(2,6-dioxo-3-piperidyl)-1,3-dioxo-isoindolin-4-yl]amino]methyl]triazol-1-yl]propanoic acid (C-3) in a similar fashion to Example 1. The residue purified by reverse phase chromatography, eluting with 5-100% MeCN in water (with 0.1% TFA modifier). LCMS (ES+): 1017.081 [M+H]⁺. ¹H NMR (400 MHz, DMSO-d₆) δ 11.08 (s, 1H), 10.09 (s, 1H), 7.99-7.94 (m, 1H), 7.63-7.60 (m, 1H), 7.57-7.53 (m, 1H), 7.52-7.47 (m, 1H), 7.29 (t, J=7.8 Hz, 1H), 7.22-7.16 (m, 1H), 7.13 (d, J=8.6 Hz, 1H), 7.10-7.05 (m, 1H), 7.04-7.00 (m, 1H), 6.90-6.86 (m, 1H), 5.05 (dd, J=12.8, 5.4 Hz, 1H), 4.91 (s, 2H), 4.63 (t, J=6.7 Hz, 2H), 4.59-4.54 (m, 2H), 4.39 (d, J=13.8 Hz, 1H), 4.27 (d, J=13.7 Hz, 1H), 3.57-3.47 (m, 1H), 3.45-3.37 (m, 1H), 3.17-3.06 (m, 1H), 3.01-2.93 (m, 2H), 2.90-2.81 (m, 2H), 2.63-2.53 (m, 1H), 2.06-1.94 (m, 1H), 1.92-1.83 (m, 1H), 1.82-1.74 (m, 1H), 1.45 (s, 3H), 1.39 (s, 3H), 1.27-1.19 (m, 3H), 1.16-1.07 (m, 1H), 1.04 (d, J=6.6 Hz, 1H).

### Example 14

### 3-(carboxymethoxy)-4-chloro-5-(3-(((4R)-1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)-2,2-dimethylpiperidin-4-yl)amino)phenyl)thiophene-2-carboxylic acid (14)

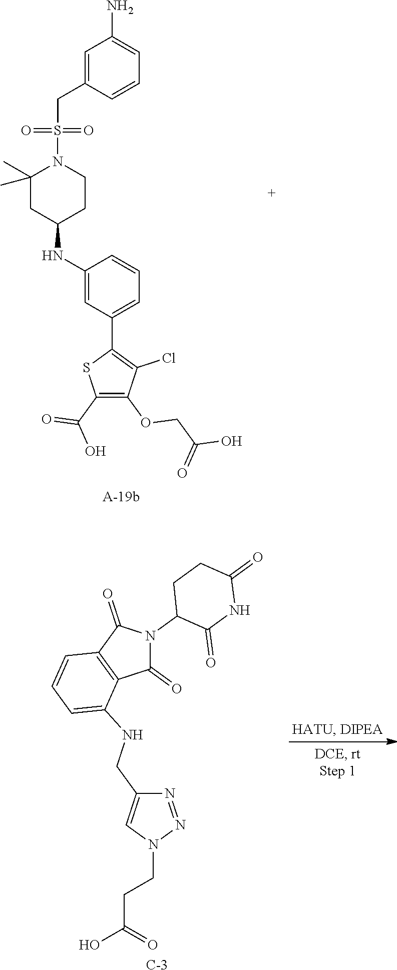

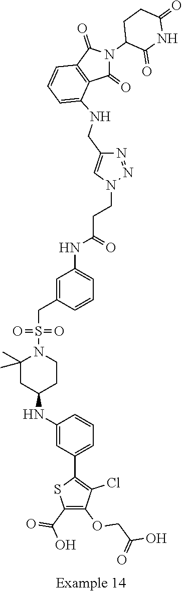 [SMILES: CC1(C)C[C@H](Nc2cccc(-c3sc(C(=O)O)c(OCC(=O)O)c3Cl)c2)CCN1S(=O)(=O)Cc1cccc(NC(=O)CCn2cc(CNc3cccc4c3C(=O)N(C3CCC(=O)NC3=O)C4=O)nn2)c1]

### Step 1: 3-(carboxymethoxy)-4-chloro-5-(3-(((4R)-1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)-2,2-dimethylpiperidin-4-yl)amino)phenyl)thiophene-2-carboxylic acid (14)

3-(carboxymethoxy)-4-chloro-5-(3-(((4R)-1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)-2,2-dimethylpiperidin-4-yl)amino)phenyl)thiophene-2-carboxylic acid (14, 4.25 mg, 10% yield) was synthesized from 5-[3-[[(4R)-1-[(3-aminophenyl)methylsulfonyl]-2,2-dimethyl-4-piperidyl]amino]phenyl]-3-(carboxymethoxy)-4-chloro-thiophene-2-carboxylic acid (A-19b) and 3-[4-[[[2-(2,6-dioxo-3-piperidyl)-1,3-dioxo-isoindolin-4-yl]amino]methyl]triazol-1-yl]propanoic acid (C-3) in a similar fashion to Example 1. The residue purified by reverse phase chromatography, eluting with 5-100% MeCN in water (with 0.1% TFA modifier). LCMS (ES+): 1016.457 [M+H]⁺.

### Example 15

3-(carboxymethoxy)-4-chloro-5-(3-(((4S)-1-((3-(3-(2-(4-(((2-(2,6-dioxopiperidin-3-yl)-1,3-dioxoisoindolin-4-yl)amino)methyl)-1H-1,2,3-triazol-1-yl)ethoxy)propanamido)benzyl)sulfonyl)-2,2-dimethylpiperidin-4-yl)amino)phenyl)thiophene-2-carboxylic acid (15)

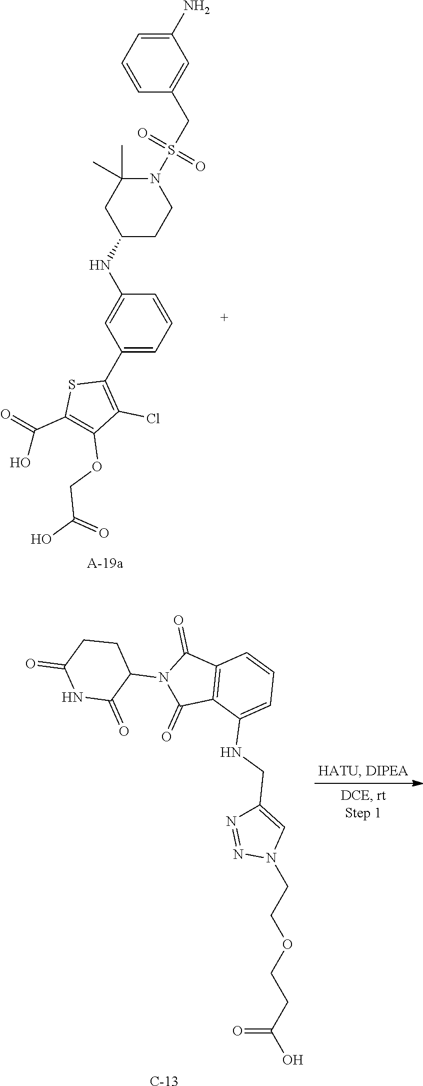

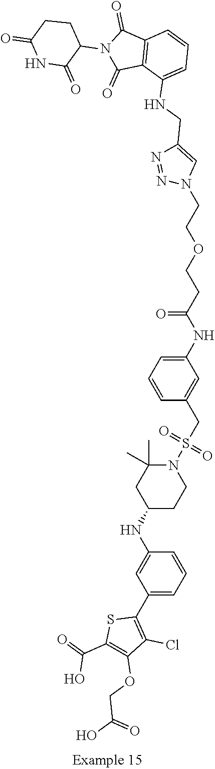 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCOCCn2cc(CNc3cccc4c3C(=O)N(C3CCC(=O)NC3=O)C4=O)nn2)c1]

### Step 1: 3-(carboxymethoxy)-4-chloro-5-(3-(((4S)-1-(3-(3-(2-(4-(2-(2,6-dioxopiperidin-3-yl)-1,3-dioxoisoindolin-4-yl))amino)methyl)-1H-1,2,3-triazol-1-yl)ethoxy)propanamido)benzyl)sulfonyl)-2,2-dimethylpiperidin-4-yl)amino)phenyl)thiophene-2-carboxylic acid (15)

3-(carboxymethoxy)-4-chloro-5-(3-(((4S)-1-((3-(3-(2-(4-(((2-(2,6-dioxopiperidin-3-yl)-1,3-dioxoisoindolin-4-yl)amino)methyl)-1H-1,2,3-triazol-1-yl)ethoxy)propanamido)benzyl)sulfonyl)-2,2-dimethylpiperidin-4-yl)amino)phenyl)thiophene-2-carboxylic acid (15, 19.98 mg, 18.65 μmol, 45% yield) was synthesized from 3-[2-[4-[[[2-(2,6-dioxo-3-piperidyl)-1,3-dioxo-isoindolin-4-yl]amino]methyl]triazol-1-yl]ethoxy]propanoic acid (C-13) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1. The residue was purified by reverse phase chromatography, eluting with 5-100% MeCN in water (with 0.1% TFA modifier). LCMS (ES+): 1060.416 [M+H]⁺. ¹H NMR (400 MHz, DMSO-d₆) δ 11.08 (s, 1H), 10.00 (s, 1H), 7.95 (s, 1H), 7.67 (s, 1H), 7.62-7.54 (m, 2H), 7.30 (t, J=7.8 Hz, 1H), 7.20 (t, J=7.9 Hz, 1H), 7.13 (d, J=8.6 Hz, 1H), 7.06 (dd, J=11.6, 7.4 Hz, 2H), 6.89-6.86 (m, 1H), 6.83-6.79 (m, 1H), 6.71-6.67 (m, 1H), 5.05 (dd, J=12.9, 5.4 Hz, 1H), 4.91 (s, 2H), 4.51-4.44 (m, 4H), 4.38 (d, J=13.7 Hz, 1H), 4.27 (d, J=13.7 Hz, 1H), 3.78 (t, J=5.2 Hz, 2H), 3.70 (t, J=6.3 Hz, 2H), 3.57-3.48 (m, 1H), 3.48-3.40 (m, 1H), 3.19-3.05 (m, 1H), 2.94-2.82 (m, 1H), 2.58 (d, J=15.9 Hz, 3H), 2.05-1.96 (m, 1H), 1.91-1.82 (m, 1H), 1.82-1.74 (m, 1H), 1.46 (s, 3H), 1.38 (s, 3H), 1.29-1.21 (m, 2H), 1.17-1.04 (m, 1H).

### 3-[2-[4-[[[2-(2,6-dioxo-3-piperidyl)-1,3-dioxo-isoindolin-4-yl]amino]methyl]triazol-1-yl]ethoxy]propanoic acid (C-13)

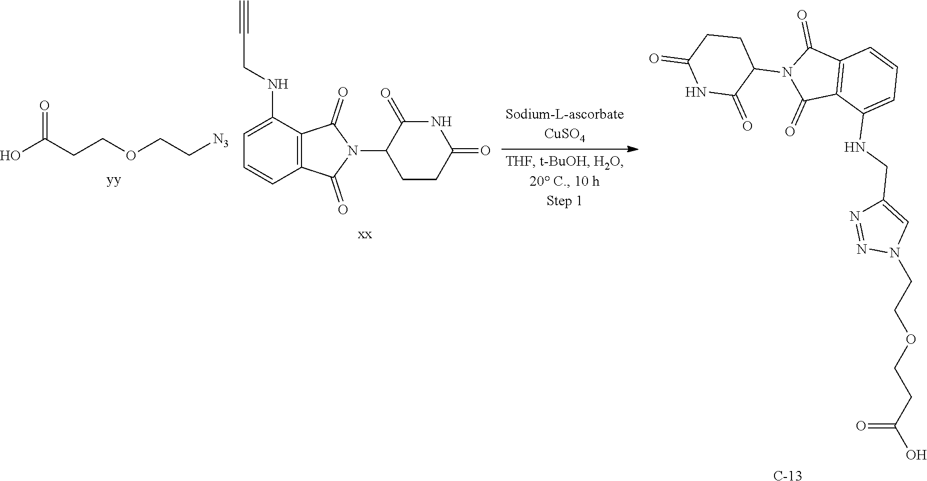

### Step 1: 3-[2-[4-[[[2-(2,6-dioxo-3-piperidyl)-1,3-dioxo-isoindolin-4-yl]amino]methyl]triazol-1-yl]ethoxy]propanoic acid (C-13)

3-[2-[4-[[2-(2,6-dioxo-3-piperidyl)-1,3-dioxo-isoindolin-4-yl]amino]methyl]triazol-1-yl]ethoxy]propanoic acid (C-13, 6.68 g, 12.78 mmol, 55% yield) was synthesized from 3-(2-azidoethoxy)propanoic acid (yy) and 2-(2,6-dioxopiperidin-3-yl)-4-(prop-2-yn-1-yl)amino)isoindoline-1,3-dione (xx) in a similar fashion to Compound C-2. LCMS (ES+): m/z 471.1 [M+H]⁺.

### Example 16

3-(carboxymethoxy)-4-chloro-5-[3-[[1-[[3-[1-[2-[[2-(2,6-dioxo-3-piperidyl)-1,3-dioxo-isoindolin-4-yl]amino]acetyl]piperidine-4-carbonyl]amino]phenyl]methylsulfonyl]-4-piperidyl]amino]phenyl]thiophene-2-carboxylic acid (16)

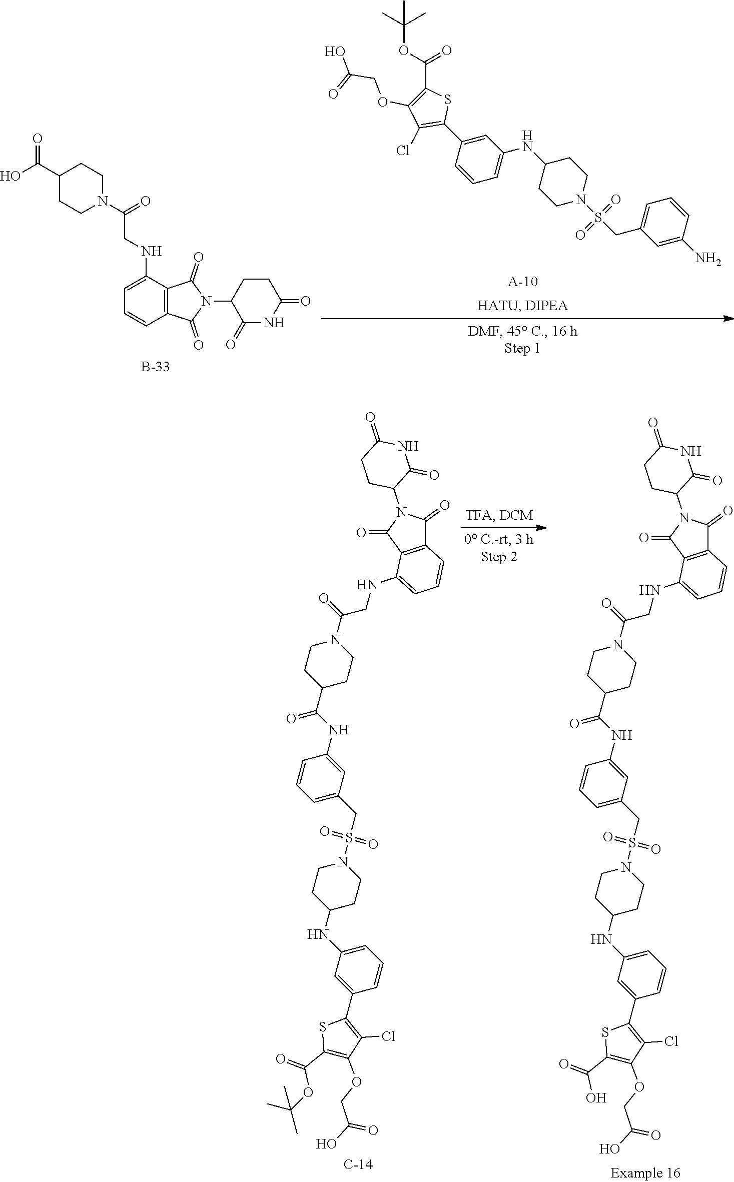

### Step 1: 2-[[2-tert-butoxycarbonyl-4-chloro-5-[3-[[1-[[3-[1-[2-[[2-(2,6-dioxo-3-piperidyl)-1,3-dioxo-isoindolin-4-yl]amino]acetyl]piperidine-4-carbonyl]amino]phenyl]methylsulfonyl]-4-piperidyl]amino]phenyl]-3-thienyl]oxy]acetic acid (C-14)

2-[[2-tert-butoxycarbonyl-4-chloro-5-[3-[[1-[[3-[1-[2-[[2-(2,6-dioxo-3-piperidyl)-1,3-dioxo-isoindolin-4-yl]amino]acetyl]piperidine-4-carbonyl]amino]phenyl]methylsulfonyl]-4-piperidyl]amino]phenyl]-3-thienyl]oxy]acetic acid (C-14, 21 mg, 19.21 μmol, 20% yield, TFA salt) was synthesized from 1-[2-[[2-(2,6-dioxo-3-piperidyl)-1,3-dioxo-isoindolin-4-yl]amino]acetyl]piperidine-4-carboxylic acid (B-33) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as a solvent. The residue was purified by reverse phase prep HPLC [Purification method: Column: SunFire-C18 100 mm, Mobile phase A: 0.1% TFA in Water, Mobile phase B: MeCN; Flow Rate: 15.0 mL/min]. LCMS (ES+): m/z 1060.7 [M+H]⁺.

### Step 2: 3-(carboxymethoxy)-4-chloro-5-[3-[[1-[[3-[1-[2-[2-(2,6-dioxo-3-piperidyl)-1,3-dioxo-isoindolin-4-yl]amino]acetyl]piperidine-4-carbonyl]amino]phenyl]methylsulfonyl]-4-piperidyl]amino]phenyl]thiophene-2-carboxylic acid (16)

3-(carboxymethoxy)-4-chloro-5-[3-[[1-[[3-[1-[2-[[2-(2,6-dioxo-3-piperidyl)-1,3-dioxo-isoindolin-4-yl]amino]acetyl]piperidine-4-carbonyl]amino]phenyl]methylsulfonyl]-4-piperidyl]amino]phenyl]thiophene-2-carboxylic acid (16, 6 mg, 5.97 μmol, 30% yield, TFA salt) was synthesized from 2-[2-tert-butoxycarbonyl-4-chloro-5-[3-[[1-[[3-[1-[2-[[2-(2,6-dioxo-3-piperidyl)-1,3-dioxo-isoindolin-4-yl]amino]acetyl]piperidine-4-carbonyl]amino]phenyl]methylsulfonyl]-4-piperidyl]amino]phenyl]-3-thienyl]oxy]acetic acid (C-14) in a similar fashion to Compound C-1, except using 200 eq. of TFA. The residue was purified by reverse phase prep HPLC [Purification method: Column: SunFire-C18 100 mm, Mobile phase A: 0.1% TFA in Water, Mobile phase B: MeCN; Flow Rate: 15.0 mL/min]. LCMS (ES+): m/z 1004.6 [M+H]⁺. ¹H NMR (400 MHz, DMSO-d₆) δ 11.10 (s, 1H), 10.04 (s, 1H), 7.71 (s, 1H), 7.64-7.54 (m, 2H), 7.31 (t, J=7.9 Hz, 1H), 7.20 (t, J=7.9 Hz, 1H), 7.14-7.04 (m, 4H), 6.89-6.85 (m, 1H), 6.80 (d, J=7.6 Hz, 1H), 6.74-6.68 (m, 1H), 5.07 (dd, J=12.9, 5.4 Hz, 1H), 4.92 (s, 2H), 4.44 (d, J=12.9 Hz, 1H), 4.37 (s, 2H), 4.20 (q, J=16.9 Hz, 2H), 3.97 (d, J=13.2 Hz, 1H), 3.17-3.04 (m, 2H), 3.02-2.83 (m, 3H), 2.81-2.71 (m, 1H), 2.67-2.54 (m, 2H), 2.10-2.00 (m, 1H), 1.99-1.91 (m, 2H), 1.90-1.83 (m, 2H), 1.76-1.45 (m, 3H), 1.42-1.30 (m, 2H), 1.29-1.20 (m, 1H).

### Example 17

### 3-(carboxymethoxy)-4-chloro-5-(3-((1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)piperidin-4-yl)(methyl)amino)phenyl)thiophene-2-carboxylic acid (17)

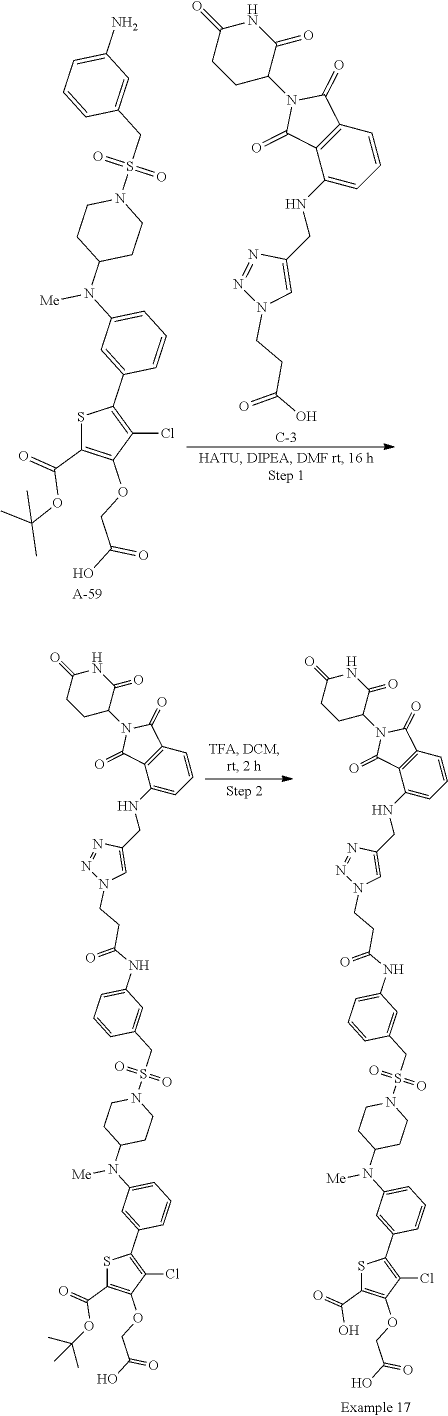

### Step 1: 2-((2-(tert-butoxycarbonyl)-4-chloro-5-(3-((1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)piperidin-4-yl)(methyl)amino)phenyl)thiophen-3-yl)oxy)acetic acid (C-15)

2-((2-(tert-butoxycarbonyl)-4-chloro-5-(3-((1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)piperidin-4-yl)(methyl)amino)phenyl)thiophen-3-yl)oxy)acetic acid (C-15, 0.025 g, 24.94 μmol, 32% yield) was synthesized from 3-[4-[[2-(2,6-dioxo-3-piperidyl)-1,3-dioxo-isoindolin-4-yl]amino]methyl]triazol-1-yl]propanoic acid (C-3) and 2-[[5-[3-[[1]-[(3-aminophenyl)methylsulfonyl]-4-piperidyl]-methyl-amino]phenyl]-2-tert-butoxycarbonyl-4-chloro-3-thienyl]oxy]acetic acid (A-59) in a similar fashion to Example 1, except using DMF as the solvent. UPLC-MS (ES+): 1058.6 [M+H]⁺.

### Step 2: 3-(carboxymethoxy)-4-chloro-5-(3-((1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)piperidin-4-yl)(methyl)amino)phenyl)thiophene-2-carboxylic acid (17)

3-(carboxymethoxy)-4-chloro-5-(3-((1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)piperidin-4-yl)(methyl)amino)phenyl)thiophene-2-carboxylic acid (17, 0.0065 g, 6.31 μmol, 27% yield, TFA salt) was synthesized from 2-((2-(tert-butoxycarbonyl)-4-chloro-5-(3-((1-((3-(3-(4-(((2-(2,6-dioxopiperidin-3-yl)-1,3-dioxoisoindolin-4-yl)amino)methyl)-1H-1,2,3-triazol-1-yl)propanamido)benzyl)sulfonyl)piperidin-4-yl)(methyl)amino)phenyl)thiophen-3-yl)oxy)acetic acid (C-15) in a similar fashion to Compound C-1. The residue was purified by reverse phase prep HPLC [Purification method: Column: SunFire C18 (19×150 mm) 5 μm, Mobile phase A: 0.1% TFA in water, Mobile phase B: MeCN; Flow Rate: 15.0 ml/min]. LCMS (ES+): m/z 1003.1 [M+H]⁺. ¹H NMR (400 MHz, DMSO-d₆) δ 11.10 (s, 1H), 10.12 (s, 1H), 8.06 (s, 1H), 7.98 (s, 1H), 7.64 (s, 1H), 7.55-7.47 (m, 2H), 7.34-7.27 (m, 2H), 7.17-6.99 (m, 5H), 6.97-6.89 (m, 2H), 5.08-5.01 (m, 1H), 4.92 (s, 2H), 4.66-4.59 (m, 2H), 4.59-4.54 (m, 2H), 4.38 (s, 2H), 3.87-3.76 (m, 1H), 3.74-3.60 (m, 4H), 3.14-3.04 (m, 2H), 3.00-2.91 (m, 4H), 2.74 (s, 3H), 1.72-1.62 (m, 2H), 1.23 (s, 2H), 1.20-1.14 (m, 2H).

### Example 18

### 3-(carboxymethoxy)-4-chloro-5-(3-(((4S)-1-((3-(7-(2-((2-(2,6-dioxopiperidin-3-yl)-1-oxoisoindolin-4-yl)amino)acetamido)heptanamido)benzyl)sulfonyl)-2,2-dimethylpiperidin-4-yl)amino)phenyl)thiophene-2-carboxylic acid (18)

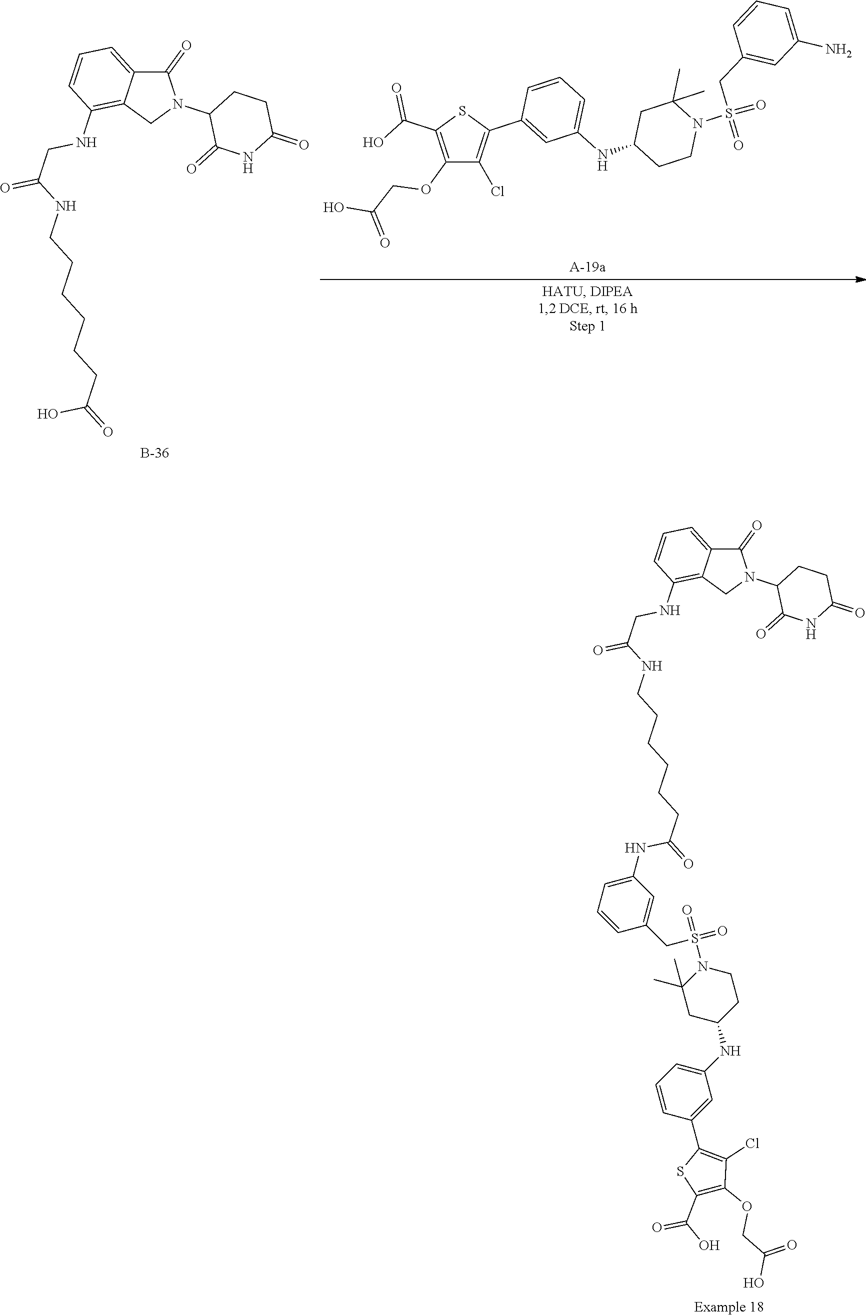

### Step 1: 3-(carboxymethoxy)-4-chloro-5-(3-(((4S)-1-((3-(7-(2-((2-(2,6-dioxopiperidin-3-yl)-1-oxoisoindolin-4-yl)amino)acetamido)heptanamido)benzyl)sulfonyl)-2,2-dimethylpiperidin-4-yl)amino)phenyl)thiophene-2-carboxylic acid (18)

3-(carboxymethoxy)-4-chloro-5-(3-(((4S)-1-((3-(7-(2-((2-(2,6-dioxopiperidin-3-yl)-1-oxoisoindolin-4-yl)amino)acetamido)heptanamido)benzyl)sulfonyl)-2,2-dimethylpiperidin-4-yl)amino)phenyl)thiophene-2-carboxylic acid (18, 9.1 mg, 8.64 μmol, 10% yield, TFA salt) was synthesized from 7-[[2-[[2-(2,6-dioxo-3-piperidyl)-1-oxo-isoindolin-4-yl]amino]acetyl]amino]heptanoic acid (B-36) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1. The residue was purified by reverse phase preparative HPLC [Purification method: SunFire Prepc18 OBD 5 μm (19×150 mm); Mobile phase A: 0.1% TFA in water and Mobile phase B: MeCN]. LCMS (ES+): m/z 1034.7 [M+H]⁺. ¹H NMR (400 MHz, DMSO-d₆) δ 11.01 (s, 1H), 9.93 (s, 1H), 7.93 (t, J=5.9 Hz, 1H), 7.66 (s, 1H), 7.58 (d, J=8.2 Hz, 1H), 7.33-7.24 (m, 2H), 7.20 (t, J=7.9 Hz, 1H), 7.08-7.03 (m, 1H), 6.97 (d, J=7.4 Hz, 1H), 6.89-6.85 (m, 1H), 6.81 (d, J=7.6 Hz, 1H), 6.72-6.66 (m, 1H), 6.55 (d, J=8.1 Hz, 1H), 5.12 (dd, J=13.3, 5.1 Hz, 1H), 4.91 (s, 2H), 4.39 (d, J=13.7 Hz, 1H), 4.32-4.14 (m, 3H), 3.72 (s, 2H), 3.09-3.01 (m, 2H), 2.97-2.85 (m, 2H), 2.65-2.59 (m, 2H), 2.50 (p, J=1.8 Hz, 184H), 2.31-2.25 (m, 2H), 2.06-1.98 (m, 1H), 1.92-1.83 (m, 1H), 1.80-1.74 (m, 1H), 1.60-1.50 (m, 2H), 1.45 (s, 3H), 1.41-1.35 (m, 4H), 1.29-1.20 (m, 4H).

### Example 19

### 3-(carboxymethoxy)-4-chloro-5-(3-((1-((3-(3-(2-(2-(3-(2-(2,6-dioxopiperidin-3-yl)-1-oxoisoindolin-5-yl)propoxy)ethoxy)ethoxy)propanamido)benzyl)sulfonyl)piperidin-4-yl)amino)phenyl)thiophene-2-carboxylic acid (19)

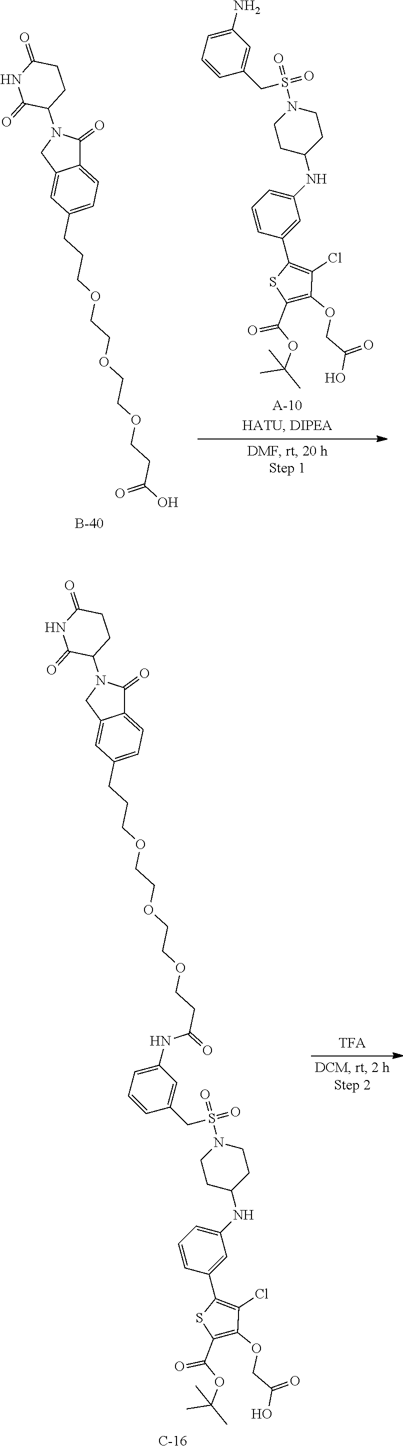

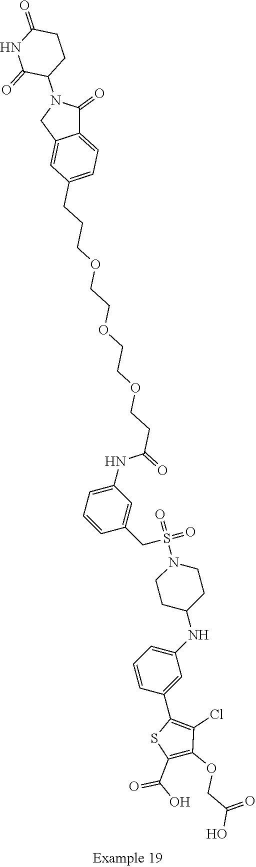 [SMILES: O=C(O)COc1c(C(=O)O)sc(-c2cccc(NC3CCN(S(=O)(=O)Cc4cccc(NC(=O)CCOCCOCCOCCCc5ccc6c(c5)CN(C5CCC(=O)NC5=O)C6=O)c4)CC3)c2)c1Cl]

### Step 1: 2-((2-(tert-butoxycarbonyl)-4-chloro-5-(3-((1-((3-(3-(2-(2-(3-(2-(2,6-dioxopiperidin-3-yl)-1-oxoisoindolin-5-yl)propoxy)ethoxy)ethoxy)propanamido)benzyl)sulfonyl)piperidin-4-yl)amino)phenyl)thiophen-3-yl)oxy)acetic acid (C-16)

2-((2-(tert-butoxycarbonyl)-4-chloro-5-(3-((1-((3-(3-(2-(2-(3-(2-(2,6-dioxopiperidin-3-yl)-1-oxoisoindolin-5-yl)propoxy)ethoxy)ethoxy)propanamido)benzyl)sulfonyl)piperidin-4-yl)amino)phenyl)thiophen-3-yl)oxy)acetic acid (C-16, 100 mg, 59.45 μmol, 61% yield) was synthesized from 3-(2-(2-(3-(2-(2,6-dioxopiperidin-3-yl)-1-oxoisoindolin-5-yl)propoxy)ethoxy)ethoxy)propanoic acid (B-40) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The material was used crude in the next step. LCMS (ES+): m/z 1080.2 [M+H]⁺.

### Step 2: 3-(carboxymethoxy)-4-chloro-5-(3-((1-((3-(3-(2-(2-(3-(2-(2,6-dioxopiperidin-3-yl)-1-oxoisoindolin-5-yl)propoxy)ethoxy)ethoxy)propanamido)benzyl)sulfonyl)piperidin-4-yl)amino)phenyl)thiophene-2-carboxylic acid (19)

3-(carboxymethoxy)-4-chloro-5-(3-((1-((3-(3-(2-(2-(3-(2-(2,6-dioxopiperidin-3-yl)-1-oxoisoindolin-5-yl)propoxy)ethoxy)ethoxy)propanamido)benzyl)sulfonyl)piperidin-4-yl)amino)phenyl)thiophene-2-carboxylic acid (19, 15 mg, 14.29 μmol, 25% yield, TFA salt) was synthesized from 2-((2-(tert-butoxycarbonyl)-4-chloro-5-(3-((1-((3-(3-(2-(2-(3-(2-(2,6-dioxopiperidin-3-yl)-1-oxoisoindolin-5-yl)propoxy)ethoxy)ethoxy)propanamido)benzyl)sulfonyl)piperidin-4-yl)amino)phenyl)thiophen-3-yl)oxy)acetic acid (C-16) in a similar fashion to Compound C-1, except using 25 eq. of TFA. The residue was purified by reverse phase preparative HPLC [Purification method: Column: SunFire prep OBD (19×50 mm), 5 μm; Mobile phase A: 0.1% TFA in water and Mobile phase B: MeCN]. UPLC (ES+): m/z 1024.8 [M+H]⁺. ¹H NMR (400 MHz, DMSO-d₆) δ 10.98 (s, 1H), 10.02 (s, 1H), 7.69-7.66 (m, 1H), 7.62 (d, J=7.8 Hz, 1H), 7.60-7.55 (m, 1H), 7.41 (s, 1H), 7.35-7.26 (m, 2H), 7.20 (t, J=7.9 Hz, 1H), 7.07 (d, J=7.7 Hz, 1H), 6.89-6.86 (m, 1H), 6.84-6.79 (m, 1H), 6.72-6.68 (m, 1H), 5.94-5.84 (m, 1H), 5.10 (dd, J=13.3, 5.1 Hz, 1H), 4.92 (s, 2H), 4.40 (d, J=17.3 Hz, 1H), 4.35 (s, 2H), 4.27 (d, J=17.3 Hz, 1H), 3.70 (t, J=6.2 Hz, 2H), 3.51 (q, J=3.8, 2.2 Hz, 9H), 3.47-3.43 (m, 3H), 3.00-2.85 (m, 4H), 2.71 (dd, J=8.7, 6.6 Hz, 2H), 2.63-2.55 (m, 3H), 2.42-2.36 (m, 1H), 2.03-1.90 (m, 3H), 1.85-1.75 (m, 2H), 1.43-1.30 (m, 2H).

### Example 20

### 3-(carboxymethoxy)-4-chloro-5-[3-[[1-[[3-[3-[2-[3-[2-(2,6-dioxo-3-piperidyl)-1,3-dioxo-isoindolin-4-yl]propoxy]ethoxy]propanoylamino]phenyl]methylsulfonyl]-4-piperidyl]amino]phenyl]thiophene-2-carboxylic acid (20)

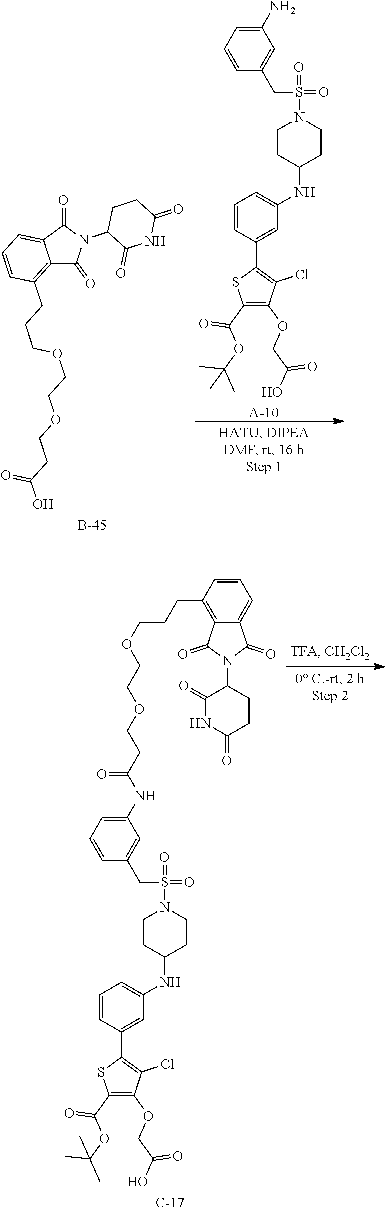

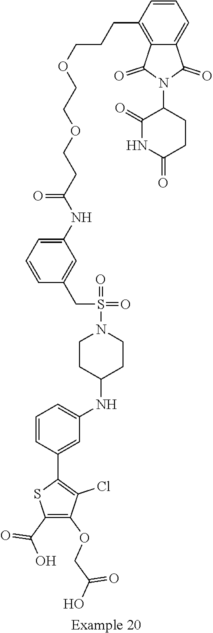

### Step 1: 2-[[2-tert-butoxycarbonyl-4-chloro-5-[3-[[1-[[3-[3-[2-[3-[2-(2,6-dioxo-3-piperidyl)-1,3-dioxo-isoindolin-4-yl]propoxy]ethoxy]propanoylamino]phenyl]methylsulfonyl]-4-piperidyl]amino]phenyl]-3-thienyl]oxy]acetic acid (C-17)

2-[[2-tert-butoxycarbonyl-4-chloro-5-[3-[[1-[[3-[3-[2-[3-[2-(2,6-dioxo-3-piperidyl)-1,3-dioxo-isoindolin-4-yl]propoxy]ethoxy]propanoylamino]phenyl]methylsulfonyl]-4-piperidyl]amino]phenyl]-3-thienyl]oxy]acetic acid (C-17, 45 mg, 40.69 μmol, 39% yield) was synthesized from 3-[2-[3-[2-(2,6-dioxo-3-piperidyl)-1,3-dioxo-isoindolin-4-yl]propoxy]ethoxy]propanoic acid (B-45) and 2-[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except with DMF as a solvent. The residue was purified by reverse phase preparative HPLC [Purification method: Column: SunFire C18 (19×150 mm) 5 μm, mobile phase: 0.1% TFA in water and MeCN]. LCMS (ES−): m/z 1048.2 [M−H]⁻.

### Step 2: 3-(carboxymethoxy)-4-chloro-5-[3-[[1-[[3-[3-[2-[3-[2-(2,6-dioxo-3-piperidyl)-1,3-dioxo-isoindolin-4-yl]propoxy]ethoxy]propanoylamino]phenyl]methylsulfonyl]-4-piperidyl]amino]phenyl]thiophene-2-carboxylic acid (20)

3-(carboxymethoxy)-4-chloro-5-[3-[[1-[[3-[3-[2-[3-[2-(2,6-dioxo-3-piperidyl)-1,3-dioxo-isoindolin-4-yl]propoxy]ethoxy]propanoylamino]phenyl]methylsulfonyl]-4-piperidyl]amino]phenyl]thiophene-2-carboxylic acid (20, 35 mg, 33.71 μmol, 79% yield, TFA salt) was synthesized from 2-[[2-tert-butoxycarbonyl-4-chloro-5-[3-[[1-[[3-[3-[2-[3-[2-(2,6-dioxo-3-piperidyl)-1,3-dioxo-isoindolin-4-yl]propoxy]ethoxy]propanoylamino]phenyl]methylsulfonyl]-4-piperidyl]amino]phenyl]-3-thienyl]oxy]acetic acid (C-17) in a similar fashion to Compound C-1, except using 300 eq. of TFA. LCMS (ES+): m/z 994.2 [M+H]⁺. ¹H-NMR (400 MHz, DMSO-d6): δ 11.12 (s, 1H), 10.02 (s, 1H), 7.74 (d, J=3.60 Hz, 2H), 7.68-7.66 (m, 2H), 7.57 (d, J=8.00 Hz, 1H), 7.29 (t, J=7.60 Hz, 1H), 7.22 (t, J=8.00 Hz, 1H), 7.07 (d, J=7.60 Hz, 1H), 6.89 (s, 1H), 6.83 (d, J=7.20 Hz, 1H), 6.72 (d, J=8.00 Hz, 1H), 5.14-5.10 (m, 1H), 4.93 (s, 2H), 4.42 (s, 2H), 3.71 (t, J=5.20 Hz, 2H), 3.56-3.38 (m, 11H), 3.03 (t, J=7.60 Hz, 2H), 2.98-2.90 (m, 3H), 2.74-2.55 (m, 7H), 2.10-2.01 (m, 1H), 1.96-1.93 (m, 2H), 1.83-1.79 (m, 2H), 1.39-1.36 (m, 2H).

### Example 21

### 3-(carboxymethoxy)-4-chloro-5-(3-((1-((3-(6-(2-((2-(2,6-dioxopiperidin-3-yl)-1-oxoisoindolin-4-yl)amino)acetamido) hexanamido)benzyl)sulfonyl)piperidin-4-yl)amino)phenyl)thiophene-2-carboxylic acid (21)

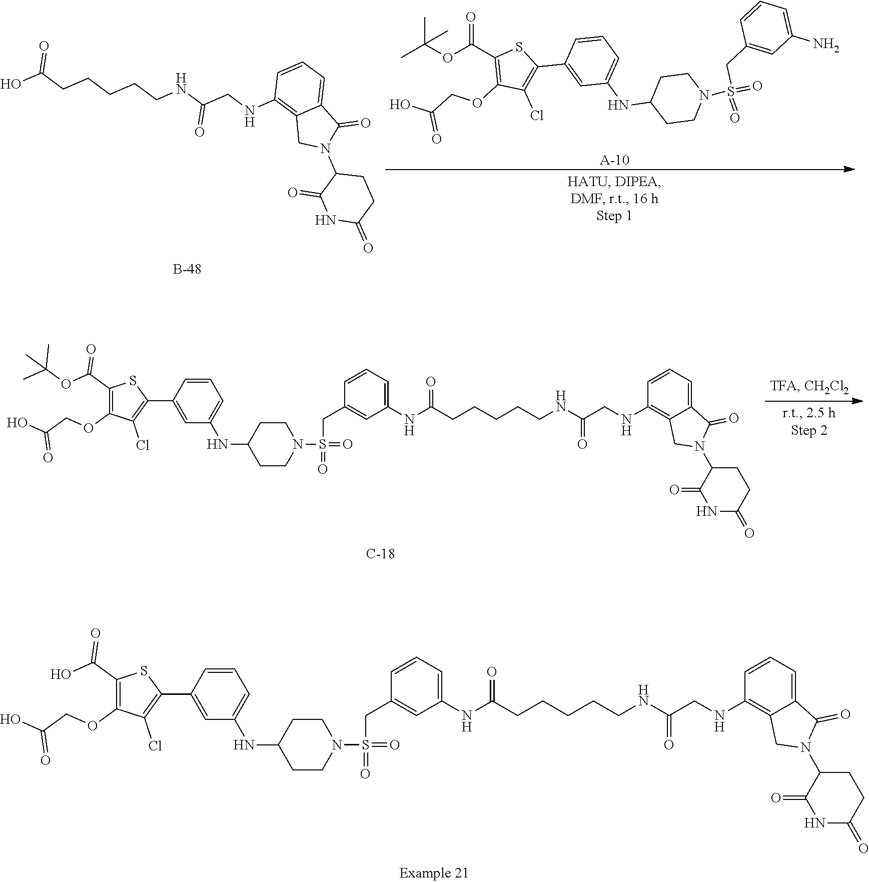

### Step 2-((2-(tert-butoxycarbonyl)-4-chloro-5-(3-((1-((3-(6-(2-((2-(2,6-dioxopiperidin-3-yl)-1-oxoisoindolin-4-yl)amino)acetamido) hexanamido)benzyl)sulfonyl)piperidin-4-yl)amino)phenyl)thiophen-3-yl)oxy)acetic acid (C-18)

2-((2-(tert-butoxycarbonyl)-4-chloro-5-(3-((1-((3-(6-(2-((2-(2,6-dioxopiperidin-3-yl)-1-oxoisoindolin-4-yl)amino)acetamido)hexanamido)benzyl)sulfonyl)piperidin-4-yl)amino)phenyl)thiophen-3-yl)oxy)acetic acid (C-18, 25 mg, 21.93 μmol, 21% yield, TFA salt) was synthesized from 6-[[2-[[2-(2,6-dioxo-3-piperidyl)-1-oxo-isoindolin-4-yl]amino]acetyl]amino]hexanoic acid (B-48) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The residue was purified by reverse phase prep HPLC [Purification method: Column: SunFire C18 (19×150 mm) 5 μm, mobile phase: 0.1% TFA in water and MeCN]. LCMS (ES+): m/z 1047.7 [M+H]⁺.

### Step 2: 3-(carboxymethoxy)-4-chloro-5-(3-((1-((3-(6-(2-((2-(2,6-dioxopiperidin-3-yl)-1-oxoisoindolin-4-yl)amino)acetamido) hexanamido)benzyl)sulfonyl)piperidin-4-yl)amino)phenyl)thiophene-2-carboxylic acid (21)

3-(carboxymethoxy)-4-chloro-5-(3-((1-((3-(6-(2-((2-(2,6-dioxopiperidin-3-yl)-1-oxoisoindolin-4-yl)amino)acetamido) hexanamido)benzyl)sulfonyl)piperidin-4-yl)amino)phenyl)thiophene-2-carboxylic acid (21, 8 mg, 7.71 μmol, 32% yield, TFA salt) was synthesized from 2-((2-(tert-butoxycarbonyl)-4-chloro-5-(3-((1-((3-(6-(2-((2-(2,6-dioxopiperidin-3-yl)-1-oxoisoindolin-4-yl)amino)acetamido) hexanamido)benzyl)sulfonyl)piperidin-4-yl)amino)phenyl)thiophen-3-yl)oxy)acetic acid (C-18) in a similar fashion to Compound C-1, except using 270 eq. of TFA. The residue was purified by reverse phase prep HPLC [Purification method: Column: SunFire C18 (19×150 mm) 5 μm, mobile phase: 0.1% TFA in water and MeCN]. LCMS (ES−): m/z 990.0 [M−H]⁻. ¹H NMR (400 MHz, DMSO-d6): δ 11.02 (s, 1H), 9.95 (s, J=Hz, 1H), 7.97-7.95 (m, 1H), 7.67 (s, 1H), 7.58 (d, J=8.60 Hz, 1H), 7.32-7.19 (m, 4H), 7.08 (d, J=7.56 Hz, 1H), 6.98 (d, J=7.36 Hz, 1H), 6.87 (s, 1H), 6.81 (d, J=7.24 Hz, 1H), 6.70 (d, J=7.64 Hz, 1H), 6.55 (d, J=8.20 Hz, 1H), 6.07 (bs, 1H), 5.89 (d, J=8.08 Hz, 1H), 5.15-5.11 (m, 1H), 4.88 (s, 2H), 4.36 (s, 2H), 4.24 (dd, J=17.00, 39.96 Hz, 2H), 3.73 (s, 2H), 3.56-3.34 (m, 3H), 3.08-2.94 (m, 8H), 2.33-2.26 (m, 4H), 2.03-1.94 (m, 4H), 1.60-1.52 (m, 3H), 1.52-1.29 (m, 6H).

### Example 22

### 3-(carboxymethoxy)-4-chloro-5-(3-((1-((3-(5-(2-((2-(2,6-dioxopiperidin-3-yl)-1-oxoisoindolin-4-yl)amino)acetamido) pentanamido)benzyl)sulfonyl)piperidin-4-yl)amino)phenyl)thiophene-2-carboxylic acid (22)

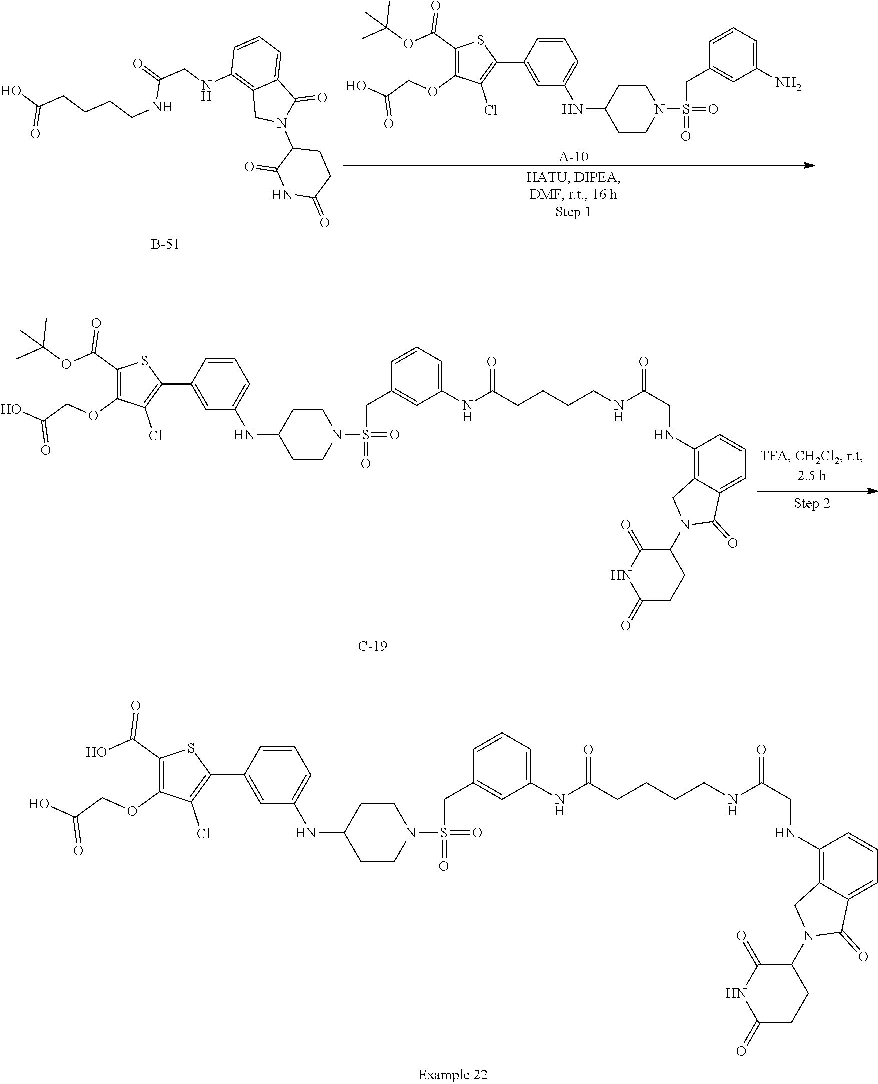

### Step 1: 2-[[2-tert-butoxycarbonyl-4-chloro-5-[3-[[1-[[3-[5-[[2-[[2-(2,6-dioxo-3-piperidyl)-1-oxo-isoindolin-4-yl]amino]acetyl]amino]pentanoylamino]phenyl]methylsulfonyl]-4-piperidyl]amino]phenyl]-3-thienyl]oxy]acetic acid (C-19)

2-[[2-tert-butoxycarbonyl-4-chloro-5-[3-[[1-[[3-[5-[[2-[[2-(2,6-dioxo-3-piperidyl)-1-oxo-isoindolin-4-yl]amino]acetyl]amino]pentanoylamino]phenyl]methylsulfonyl]-4-piperidyl]amino]phenyl]-3-thienyl]oxy]acetic acid (C-19, 30 mg, 27.84 μmol, 17% yield, TFA salt) was synthesized from 5-[[2-[[2-(2,6-dioxo-3-piperidyl)-1-oxo-isoindolin-4-yl]amino]acetyl]amino]pentanoic acid (B-51) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The residue was purified by reverse phase prep HPLC [Purification method: Column: SunFire C18 (19×150 mm) 5 μm, mobile phase: 0.1% TFA in water and MeCN]. LCMS (ES+): m/z 1033.7 [M+H]⁺.

### Step 2: 3-(carboxymethoxy)-4-chloro-5-(3-((1-((3-(5-(2-((2-(2,6-dioxopiperidin-3-yl)-1-oxoisoindolin-4-yl)amino)acetamido) pentanamido)benzyl)sulfonyl)piperidin-4-yl)amino)phenyl)thiophene-2-carboxylic acid (22)

3-(carboxymethoxy)-4-chloro-5-(3-((1-((3-(5-(2-((2-(2,6-dioxopiperidin-3-yl)-1-oxoisoindolin-4-yl)amino)acetamido) pentanamido)benzyl)sulfonyl)piperidin-4-yl)amino)phenyl)thiophene-2-carboxylic acid (22, 8.34 mg, 8.01 μmol, 28% yield, TFA salt) was synthesized from 2-[[2-tert-butoxycarbonyl-4-chloro-5-[3-[[1-[[3-[5-[[2-[[2-(2,6-dioxo-3-piperidyl)-1-oxo-isoindolin-4-yl]amino]acetyl]amino]pentanoylamino]phenyl]methylsulfonyl]-4-piperidyl]amino]phenyl]-3-thienyl]oxy]acetic acid (C-19) in a similar fashion to Compound C-1, except using 224 eq. of TFA. The residue was purified by reverse phase prep HPLC [Purification method: Column: SunFire C18 (19×150 mm) 5 μm, mobile phase: 0.1% TFA in water and MeCN]. LCMS (ES+): m/z 978.1 [M+H]⁺. ¹H NMR (400 MHz, DMSO-d₆) δ 11.01 (s, 1H), 9.95 (s, 1H), 7.99 (t, J=5.9 Hz, 1H), 7.66 (s, 1H), 7.57 (d, J=8.2 Hz, 1H), 7.34-7.24 (m, 2H), 7.20 (t, J=7.9 Hz, 1H), 7.07 (d, J=7.4 Hz, 1H), 6.96 (d, J=7.5 Hz, 1H), 6.87 (s, 1H), 6.81 (d, J=7.6 Hz, 1H), 6.70 (d, J=8.3 Hz, 1H), 6.55 (d, J=8.1 Hz, 1H), 5.89 (s, 1H), 5.12 (dd, J=13.2, 5.1 Hz, 1H), 4.92 (s, 2H), 4.36 (s, 2H), 4.28 (d, J=17.1 Hz, 1H), 4.18 (d, J=17.0 Hz, 1H), 3.72 (s, 2H), 3.54 (d, J=11.9 Hz, 3H), 3.09 (q, J=6.6 Hz, 2H), 3.01-2.87 (m, 4H), 2.65-2.57 (m, 2H), 2.29 (t, J=7.1 Hz, 2H), 2.06-1.90 (m, 4H), 1.60-1.49 (m, 2H), 1.47-1.31 (m, 4H).

### Example 23

### 3-(carboxymethoxy)-4-chloro-5-[3-[[1-[[3-[[2-[[2-(2,6-dioxo-3-piperidyl)-1,3-dioxo-isoindolin-5-yl]amino]acetyl]amino]phenyl]methylsulfonyl]-4-piperidyl]amino]phenyl]thiophene-2-carboxylic acid (23)

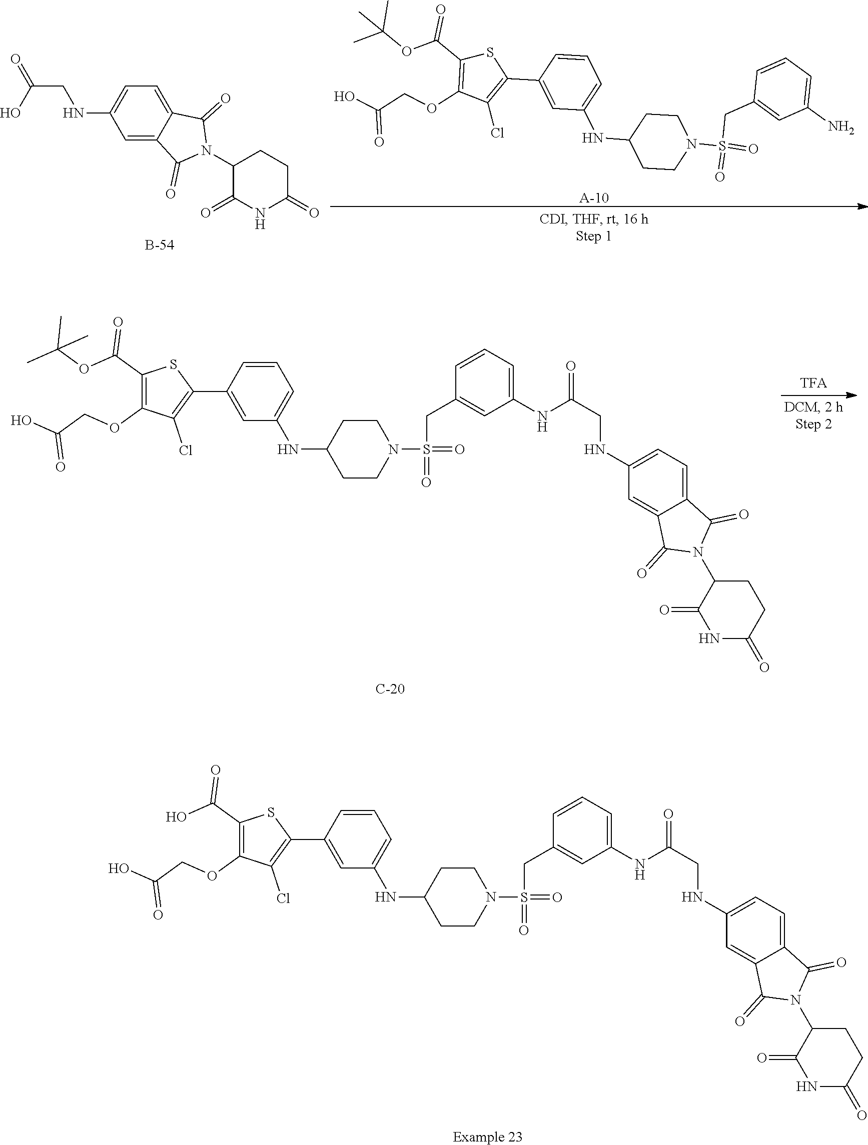

### Step 1: 2-[[2-tert-butoxycarbonyl-4-chloro-5-[3-[[1-[[3-[[2-[[2-(2,6-dioxo-3-piperidyl)-1,3-dioxo-isoindolin-S-yl]amino]acetyl]amino]phenyl]methylsulfonyl]-4-piperidyl]amino]phenyl]-3-thienyl]oxy]acetic acid (C-20)

Into a 10 mL single neck round bottom flask containing a well-stirred solution of 2-[[2-(2,6-dioxo-3-piperidyl)-1,3-dioxo-isoindolin-5-yl]amino]acetic acid (B-54, 50.00 mg, 150.93 μmol) in anhydrous THF (2 mL) was added carbonyl diimidazole (32.62 mg, 201.17 μmol) at room temperature. The resultant reaction mixture was stirred at room temperature for 2 h. Then a solution of 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10, 76.81 mg, 120.74 μmol) in anhydrous THF (2 mL) was added. The reaction mixture was stirred for 14 h at room temperature. After completion of reaction, volatiles were evaporated under reduced pressure to afford 2-[2-tert-butoxycarbonyl-4-chloro-5-[3-[[1-[[3-[[2-[[2-(2,6-dioxo-3-piperidyl)-1,3-dioxo-isoindolin-5-yl]amino]acetyl]amino]phenyl]methylsulfonyl]-4-piperidyl]amino]phenyl]-3-thienyl]oxy]acetic acid (C-20, 0.030 g, 6.32 μmol, 4% yield) as a yellow solid. LCMS (ES+): m/z 950.3 [M+H]⁺

### Step 2: 3-(carboxymethoxy)-4-chloro-5-[3-[[1-[[3-[[2-[[2-(2,6-dioxo-3-piperidyl)-1,3-dioxo-isoindolin-5-yl]amino]acetyl]amino]phenyl]methylsulfonyl]-4-piperidyl]amino]phenyl]thiophene-2-carboxylic acid (23)

3-(carboxymethoxy)-4-chloro-5-[3-[[1-[[3-[[2-[[2-(2,6-dioxo-3-piperidyl)-1,3-dioxo-isoindolin-5-yl]amino]acetyl]amino]phenyl]methylsulfonyl]-4-piperidyl]amino]phenyl]thiophene-2-carboxylic acid (23, 0.0073 g, 8.12 μmol, 32% yield, TFA salt) was synthesized from 2-[[2-tert-butoxycarbonyl-4-chloro-5-[3-[[1-[[3-[[2-[[2-(2,6-dioxo-3-piperidyl)-1,3-dioxo-isoindolin-5-yl]amino]acetyl]amino]phenyl]methylsulfonyl]-4-piperidyl]amino]phenyl]-3-thienyl]oxy]acetic acid (C-20) in a similar fashion to Compound C-1. The residue was purified by reverse phase prep HPLC [Purification method: Column: SunFire C18 (19×150 mm) 5 μm, Mobile phase A: 0.1% TFA in water, Mobile phase B: MeCN; Flow Rate: 15.0 ml/min]. LCMS (ES−): m/z 891.2 [M−H]⁻. ¹H NMR (400 MHz, DMSO-d6): δ 11.07 (s, 1H), 10.25 (s, 1H), 7.67-7.59 (m, 3H), 6.98-6.91 (m, 1H), 7.34 (t, J=7.88 Hz, 1H), 7.20 (t, J=7.88 Hz, 1H), 7.13-7.09 (m, 1H), 7.03 (s, 1H), 6.96-6.94 (m, 1H), 6.92 (s, 1H), 6.85 (d, J=18.84 Hz, 1H), 6.75 (d, J=41.36 Hz, 1H), 5.89 (bs, 1H), 5.04 (dd, J=5.44, 12.82 Hz, 1H), 4.91 (s, 2H), 4.55 (bs, 1H), 4.38 (s, 2H), 4.12 (d, J=5.24 Hz, 2H), 3.57-3.54 (m, 3H), 3.51-3.48 (m, 3H), 3.43-3.41 (m, 6H), 2.96 (t, J=10.52 Hz, 2H), 2.90-2.82 (m, 1H), 2.65-2.55 (m, 2H), 2.00-1.94 (m, 4H), 1.38-1.36 (m, 2H).

### Example 24

### 3-(carboxymethoxy)-4-chloro-5-[3-[[1-[[3-[3-[2-[[2-[[2-(2,6-dioxo-3-piperidyl)-1,3-dioxo-isoindolin-5-yl]amino]acetyl]amino]ethoxy]propanoylamino]phenyl]methylsulfonyl]-4-piperidyl]amino]phenyl]thiophene-2-carboxylic acid (24)

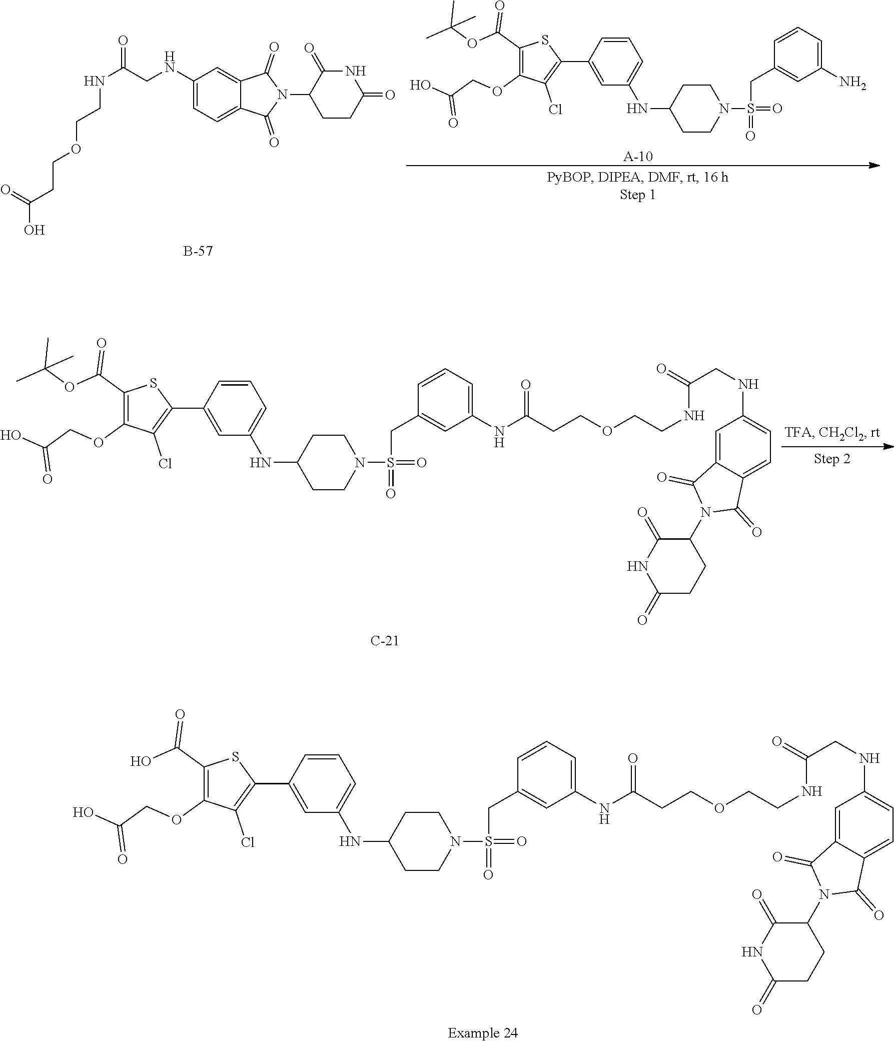

### Step 1: 2-[[2-tert-butoxycarbonyl-4-chloro-5-[3-[[1-[[3-[3-[2-[[2-[[2-(2,6-dioxo-3-piperidyl)-1,3-dioxo-isoindolin-5-yl]amino]acetyl]amino]ethoxy]propanoylamino]phenyl]methylsulfonyl]-4-piperidyl]amino]phenyl]-3-thienyl]oxy]acetic acid (C-21)

Into a 10 mL single neck round bottom flask containing a well-stirred solution of 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10, 50 mg, 78.59 μmol) in anhydrous DMF (2 mL) were added 3-[2-[[2-[[2-(2,6-dioxo-3-piperidyl)-1,3-dioxo-isoindolin-5-yl]amino]acetyl]amino]ethoxy]propanoic acid (B-57, 42.10 mg, 94.31 μmol) and DIPEA (10.16 mg, 78.59 μmol, 13.69 μL) followed by PyBOP (44.99 mg, 86.45 μmol). The reaction mixture was stirred at room temperature for 16 h, at which point LCMS indicated complete consumption of starting material. The reaction mixture was diluted with water (5 mL) and 10% MeOH in DCM (20 mL). The organic layer was separated and washed with brine (5 mL), dried over Sodium sulfate and solvent removed. The crude compound was purified by Prep-HPLC [Prep Method: Column: SunFire C18 (19×150 mm) 5 μm, Mobile phase A: 0.1% TFA in water:MeCN] to afford 2-[[2-tert-butoxycarbonyl-4-chloro-5-[3-[[1-[[3-[3-[2-[[2-[[2-(2,6-dioxo-3-piperidyl)-1,3-dioxo-isoindolin-5-yl]amino]acetyl]amino]ethoxy]propanoylamino]phenyl]methylsulfonyl]-4-piperidyl]amino]phenyl]-3-thienyl]oxy]acetic acid (C-21, 15 mg, 12.96 μmol, 16% yield, TFA salt) as a yellow solid. LCMS (ES+): m/z 1064.3 [M+H]⁺.

### Step 2: 3-(carboxymethoxy)-4-chloro-5-[3-[[1-[[3-[3-[2-[[2-[[2-(2,6-dioxo-3-piperidyl)-1,3-dioxo-isoindolin-5-yl]amino]acetyl]amino]ethoxy]propanoylamino]phenyl]methylsulfonyl]-4-piperidyl]amino]phenyl]thiophene-2-carboxylic acid (24)

3-(carboxymethoxy)-4-chloro-5-[3-[[1-[[3-[3-[2-[2-[[2-(2,6-dioxo-3-piperidyl)-1,3-dioxo-isoindolin-5-yl]amino]acetyl]amino]ethoxy]propanoylamino]phenyl]methylsulfonyl]-4-piperidyl]amino]phenyl]thiophene-2-carboxylic acid (24, 9 mg, 8.74 μmol, 62% yield, TFA salt) was synthesized from 2-[[2-tert-butoxycarbonyl-4-chloro-5-[3-[[1-[[3-[3-[2-[[2-[[2-(2,6-dioxo-3-piperidyl)-1,3-dioxo-isoindolin-5-yl]amino]acetyl]amino]ethoxy]propanoylamino]phenyl]methylsulfonyl]-4-piperidyl]amino]phenyl]-3-thienyl]oxy]acetic acid (C-21) in a similar fashion to Compound C-1, except using 3 eq. of TFA. The residue was purified by reverse phase prep HPLC [Prep Method: Column: SunFire C18 (19×150 mm) 5 μm; Mobile phase: 0.1% TFA in water and MeCN]. LCMS (ES+): m/z 1010.3 [M+H]⁺. ¹H NMR (400 MHz, DMSO-d₆) δ 11.06 (s, 1H), 10.02 (s, 1H), 8.14-8.07 (m, 1H), 7.67 (s, 1H), 7.58 (d, J=8.2 Hz, 2H), 7.36-7.27 (m, 2H), 7.21 (t, J=7.9 Hz, 1H), 7.11-7.05 (m, 1H), 6.93 (s, 1H), 6.89-6.78 (m, 3H), 6.70 (d, J=8.3 Hz, 1H), 5.89 (s, 1H), 5.03 (dd, J=13.1, 5.5 Hz, 1H), 4.92 (s, 2H), 4.35 (s, 2H), 3.83 (s, 3H), 3.71-3.66 (m, 2H), 3.60-3.51 (m, 4H), 3.01-2.80 (m, 5H), 2.07 (s, 1H), 2.04-1.91 (m, 4H), 1.49-1.30 (m, 3H), 1.23 (s, 1H).

### Example 25

### 3-(carboxymethoxy)-4-chloro-5-[3-[[1-[[3-[7-[[2-[[2-(2,6-dioxo-3-piperidyl)-1-oxo-isoindolin-5-yl]amino]acetyl]amino]heptanoylamino]phenyl]methylsulfonyl]-4-piperidyl]amino]phenyl]thiophene-2-carboxylic acid (25)

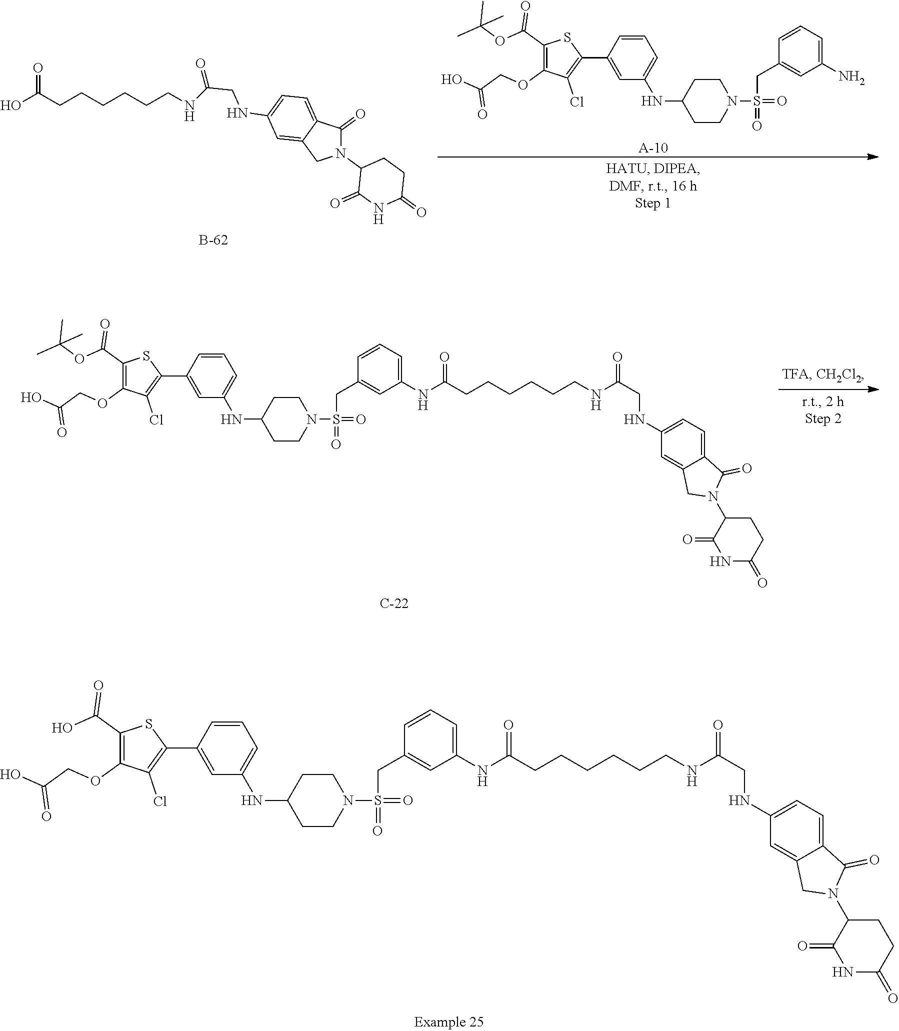

### Step 1: 2-[[2-tert-butoxycarbonyl-4-chloro-5-[3-[[1-[[3-[7-[[2-[[2-(2,6-dioxo-3-piperidyl)-1-oxo-isoindolin-5-yl]amino]acetyl]amino]heptanoylamino]phenyl]methylsulfonyl]-4-piperidyl]amino]phenyl]-3-thienyl]oxy]acetic acid (C-22)

2-[[2-tert-butoxycarbonyl-4-chloro-5-[3-[[1-[[3-[7-[[2-[[2-(2,6-dioxo-3-piperidyl)-1-oxo-isoindolin-5-yl]amino]acetyl]amino]heptanoylamino]phenyl]methylsulfonyl]-4-piperidyl]amino]phenyl]-3-thienyl]oxy]acetic acid (C-22, 30 mg, 26.63 μmol, 24% yield) was synthesized from 7-[[2-[[2-(2,6-dioxo-3-piperidyl)-1-oxo-isoindolin-5-yl]amino]acetyl]amino]heptanoic acid (B-62) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. LCMS (ES+): m/z 1062.3 [M+H]⁺.

### Step 2: 3-(carboxymethoxy)-4-chloro-5-[3-[[1-[[3-[7-[[2-[[2-(2,6-dioxo-3-piperidyl)-1-oxo-isoindolin-5-yl]amino]acetyl]amino]heptanoylamino]phenyl]methylsulfonyl]-4-piperidyl]amino]phenyl]thiophene-2-carboxylic acid (25)

3-(carboxymethoxy)-4-chloro-5-[3-[[1-[[3-[7-[[2-[[2-(2,6-dioxo-3-piperidyl)-1-oxo-isoindolin-5-yl]amino]acetyl]amino]heptanoylamino]phenyl]methylsulfonyl]-4-piperidyl]amino]phenyl]thiophene-2-carboxylic acid (25, 5.51 mg, 5.07 μmol, 18% yield, TFA salt) was synthesized from 2-[[2-tert-butoxycarbonyl-4-chloro-5-[3-[[1-[[3-[7-[[2-[[2-(2,6-dioxo-3-piperidyl)-1-oxo-isoindolin-5-yl]amino]acetyl]amino]heptanoylamino]phenyl]methylsulfonyl]-4-piperidyl]amino]phenyl]-3-thienyl]oxy]acetic acid (C-22) in a similar fashion to Compound C-1, except using 230 eq. of TFA. The residue was purified by reverse phase prep HPLC [Purification method: Column: Sun fire C18 (19×150 MM) 5.0 μm; Mobile phase A: 0.1% TFA in water and mobile phase B: MeCN]. LCMS (ES+): m/z 1006.2 [M+H]⁺. ¹H NMR (400 MHz, DMSO-d₆) δ 10.92 (s, 1H), 9.94 (s, 1H), 7.91 (t, J=5.8 Hz, 1H), 7.66 (s, 1H), 7.55 (d, J=8.1 Hz, 1H), 7.41 (d, J=8.4 Hz, 1H), 7.33-7.26 (m, 1H), 7.21 (t, J=7.9 Hz, 1H), 7.10-7.05 (m, 1H), 6.88 (s, 1H), 6.83 (d, J=7.6 Hz, 1H), 6.71 (d, J=8.3 Hz, 1H), 6.66 (d, J=8.2 Hz, 1H), 6.58 (s, 1H), 5.00 (dd, J=13.4, 5.1 Hz, 1H), 4.92 (s, 2H), 4.35 (s, 2H), 4.27 (d, J=16.8 Hz, 1H), 4.14 (d, J=16.8 Hz, 1H), 3.70 (s, 3H), 3.09-3.03 (m, 3H), 3.02-2.91 (m, 3H), 2.91-2.81 (m, 2H), 2.38-2.24 (m, 4H), 1.99-1.90 (m, 3H), 1.60-1.50 (m, 2H), 1.43-1.32 (m, 4H), 1.31-1.21 (m, 4H).

### Example 26

### 3-(carboxymethoxy)-4-chloro-5-[3-[[1-[[3-[7-[2-[[2-(2,6-dioxo-3-piperidyl)-1-oxo-isoindolin-4-yl]amino]propanoylamino]heptanoylamino]phenyl]methylsulfonyl]-4-piperidyl]amino]phenyl]thiophene-2-carboxylic acid (26)

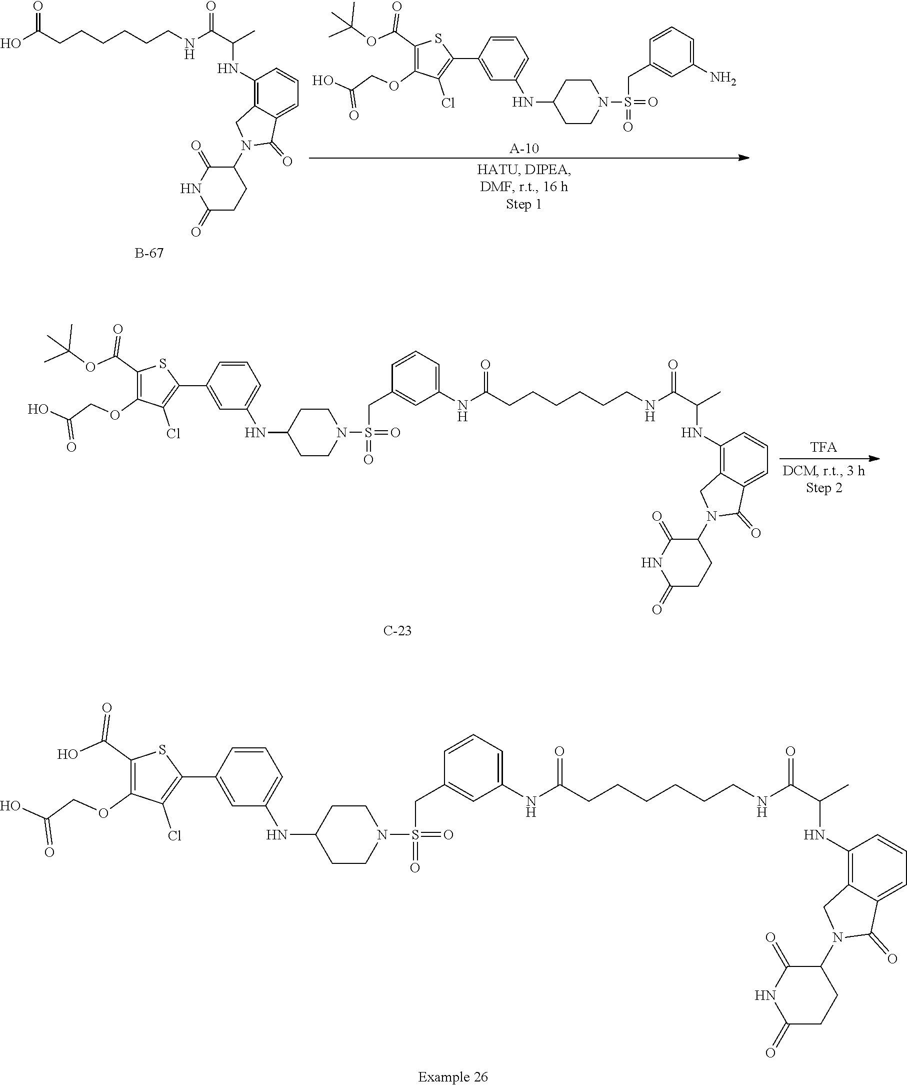

### Step 1: 2-[[2-tert-butoxycarbonyl-4-chloro-5-[3-[[1-[[3-[7-[[2-[[2-(2,6-dioxo-3-piperidyl)-1-oxo-isoindolin-4-yl]amino]propanoylamino]heptanoylamino]phenyl]methylsulfonyl]-4-piperidyl]amino]phenyl]-3-thienyl]oxy]acetic acid (C-23)

A mixture of diastereomers of 2-[[2-tert-butoxycarbonyl-4-chloro-5-[3-[[1-[[3-[7-[[2-[[2-(2,6-dioxo-3-piperidyl)-1-oxo-isoindolin-4-yl]amino]propanoylamino]heptanoylamino]phenyl]methylsulfonyl]-4-piperidyl]amino]phenyl]-3-thienyl]oxy]acetic acid (C-23, 30 mg, 27.79 μmol, 25% yield, TFA salt) were synthesized from 7-[2-[[2-(2,6-dioxo-3-piperidyl)-1-oxo-isoindolin-4-yl]amino]propanoylamino]heptanoic acid (B-67) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The residue was purified by reverse phase prep HPLC [Purification method: XSelect (150×19 mm) 5 μm; Mobile phase A: 0.1% TFA in water and mobile phase B: MeCN]. LCMS (ES−): m/z 1074.3 [M−H]⁻.

### Step 2: 3-(carboxymethoxy)-4-chloro-5-[3-[[1-[[3-[7-[[2-[[2-(2,6-dioxo-3-piperidyl)-1-oxo-isoindolin-4-yl]amino]propanoylamino]heptanoylamino]phenyl]methylsulfonyl]-4-piperidyl]amino]phenyl]thiophene-2-carboxylic acid (26)

A mixture of diastereomers of 3-(carboxymethoxy)-4-chloro-5-[3-[[1-[[3-[7-[2-[[2-(2,6-dioxo-3-piperidyl)-1-oxo-isoindolin-4-yl]amino]propanoylamino]heptanoylamino]phenyl]methylsulfonyl]-4-piperidyl]amino]phenyl]thiophene-2-carboxylic acid (26, 21.5 mg, 19.86 μmol, 71% yield, TFA salt) were synthesized from 2-[[2-tert-butoxycarbonyl-4-chloro-5-[3-[[1-[[3-[7-[[2-[[2-(2,6-dioxo-3-piperidyl)-1-oxo-isoindolin-4-yl]amino]propanoylamino]heptanoylamino]phenyl]methylsulfonyl]-4-piperidyl]amino]phenyl]-3-thienyl]oxy]acetic acid (C-23) in a similar fashion to Compound C-1, except using 230 eq. of TFA. The residue was purified by reverse phase prep HPLC [Purification method: SunFire C₁₈ column (19×150 mm) 5 μm; Mobile phase A: 0.1% TFA in water and mobile phase B: MeCN]. LCMS (ES+): m/z 1020.2 [M+H]⁺. ¹H-NMR (400 MHz, DMSO-d6): δ 11.04 (d, J=5.16 Hz, 1H), 9.95 (s, 1H), 7.99-7.91 (m, 1H), 7.68 (s, 1H), 7.57 (d, J=8.76 Hz, 1H), 7.32-7.19 (m, 3H), 7.08 (d, J=7.72 Hz, 1H), 6.98 (d, J=7.48 Hz, 1H), 6.88 (s, 1H), 6.82 (d, J=7.72 Hz, 1H), 6.72-6.59 (m, 1H), 6.58 (d, J=8.00 Hz, 1H), 5.17-5.11 (m, 1H), 4.92 (s, 2H), 4.37-4.16 (m, 3H), 3.90-3.89 (m, 1H), 3.03-2.90 (m, 6H), 2.68-2.61 (m, 3H), 2.10-1.90 (m, 3H), 1.56-1.52 (m, 2H), 1.41-1.24 (m, 7H), 1.24-1.08 (m, 8H).

### Example 27

### 3-(carboxymethoxy)-4-chloro-5-[3-[[1-[[3-[3-[3-[2-(2,6-dioxo-3-piperidyl)-1,3-dioxo-isoindolin-4-yl]propoxy]propanoylamino]phenyl]methylsulfonyl]-4-piperidyl]amino]phenyl]thiophene-2-carboxylic acid (27)

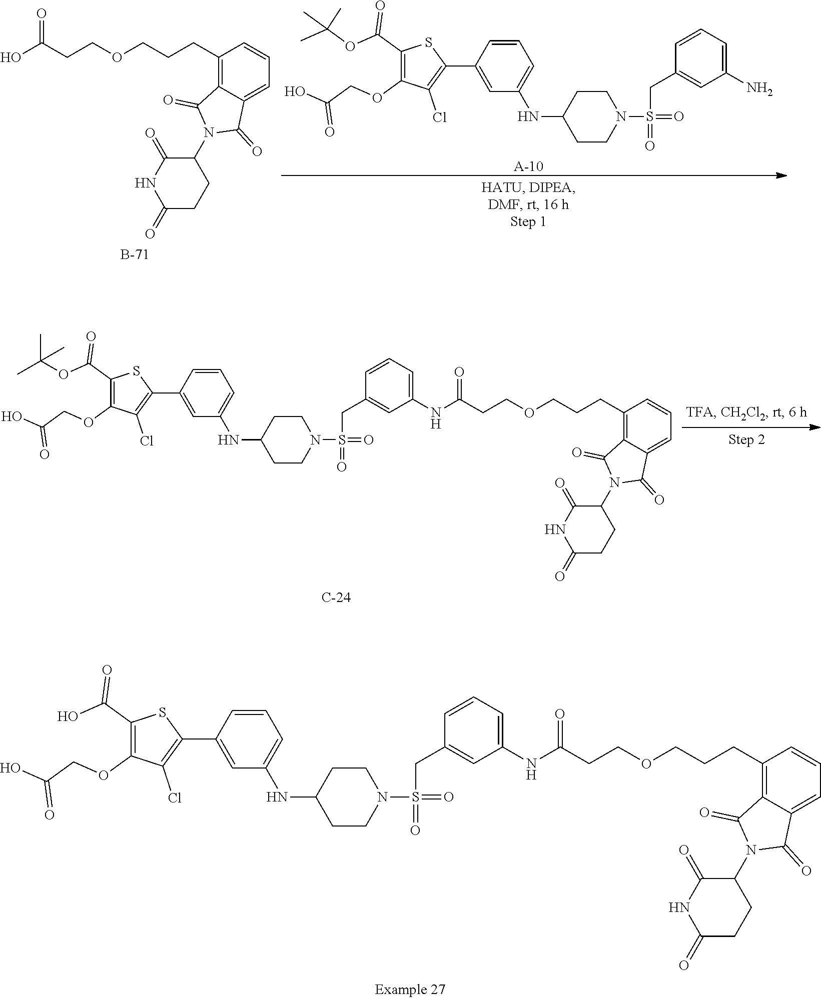

### Step 1: 2-[[2-tert-butoxycarbonyl-4-chloro-5-[3-[[1-[[3-[3-[3-[2-(2,6-dioxo-3-piperidyl)-1,3-dioxo-isoindolin-4-yl]propoxy]propanoylamino]phenyl]methylsulfonyl]-4-piperidyl]amino]phenyl]-3-thienyl]oxy]acetic acid (C-24)

2-[[2-tert-butoxycarbonyl-4-chloro-5-[3-[[1-[[3-[3-[3-[2-(2,6-dioxo-3-piperidyl)-1,3-dioxo-isoindolin-4-yl]propoxy]propanoylamino]phenyl]methylsulfonyl]-4-piperidyl]amino]phenyl]-3-thienyl]oxy]acetic acid (C-24, 21 mg, 20.45 μmol, 13% yield, TFA salt) was synthesized from 3-[3-[2-(2,6-dioxo-3-piperidyl)-1,3-dioxo-isoindolin-4-yl]propoxy]propanoic acid (B-71) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The residue was purified by reverse phase prep HPLC [Purification method: Column: SunFire-C18, 100 mm, Mobile phase A: 0.1% TFA in water, Mobile phase B: MeCN; Flow Rate: 15.0 mL/min]. LCMS (ES+): m/z 1006.7 [M+H]⁺.

### Step 2: 3-(carboxymethoxy)-4-chloro-5-[3-[[1-[[3-[3-[3-[2-(2,6-dioxo-3-piperidyl)-1,3-dioxo-isoindolin-4-yl]propoxy]propanoylamino]phenyl]methylsulfonyl]-4-piperidyl]amino]phenyl]thiophene-2-carboxylic acid (27)

3-(carboxymethoxy)-4-chloro-5-[3-[[1-[[3-[3-[3-[2-(2,6-dioxo-3-piperidyl)-1,3-dioxo-isoindolin-4-yl]propoxy]propanoylamino]phenyl]methylsulfonyl]-4-piperidyl]amino]phenyl]thiophene-2-carboxylic acid (27, 12.68 mg, 13.03 μmol, 66% yield, TFA salt) was synthesized from 2-[[2-tert-butoxycarbonyl-4-chloro-5-[3-[[1-[[3-[3-[3-[2-(2,6-dioxo-3-piperidyl)-1,3-dioxo-isoindolin-4-yl]propoxy]propanoylamino]phenyl]methylsulfonyl]-4-piperidyl]amino]phenyl]-3-thienyl]oxy]acetic acid (C-24) in a similar fashion to Compound C-1, except using 5 eq. of TFA. The residue was purified by reverse phase prep HPLC [Purification method: Column: SunFire-C18 100 mm, Mobile phase A: 0.1% TFA in water, Mobile phase B: MeCN; Flow Rate: 15.0 mL/min]. LCMS (ES+): m/z 949.6 [M+H]⁺. ¹H NMR (400 MHz, DMSO-d₆) δ 11.11 (s, 1H), 10.04 (s, 1H), 7.74-7.70 (m, 2H), 7.70-7.68 (m, 1H), 7.64 (dd, J=6.3, 2.5 Hz, 1H), 7.61-7.56 (m, 1H), 7.30 (t, J=7.9 Hz, 1H), 7.20 (t, J=7.9 Hz, 1H), 7.08 (d, J=7.8 Hz, 1H), 6.90-6.86 (m, 1H), 6.84-6.79 (m, 1H), 6.74-6.67 (m, 1H), 5.11 (dd, J=12.9, 5.4 Hz, 1H), 4.92 (s, 2H), 4.36 (s, 2H), 3.40 (t, J=6.4 Hz, 3H), 3.03 (t, J=7.6 Hz, 2H), 2.95 (t, J=11.1 Hz, 2H), 2.90-2.82 (m, 1H), 2.63-2.55 (m, 4H), 2.07-1.99 (m, 1H), 1.97-1.90 (m, 2H), 1.87-1.77 (m, 2H), 1.43-1.29 (m, 2H).

### Example 28

### 3-(carboxymethoxy)-4-chloro-5-(3-(((4S)-1-((3-(4-(2-((2-(2,6-dioxopiperidin-3-yl)-1-oxoisoindolin-4-yl)amino)acetamido) butanamido)benzyl)sulfonyl)-2,2-dimethylpiperidin-4-yl)amino)phenyl)thiophene-2-carboxylic acid (28)

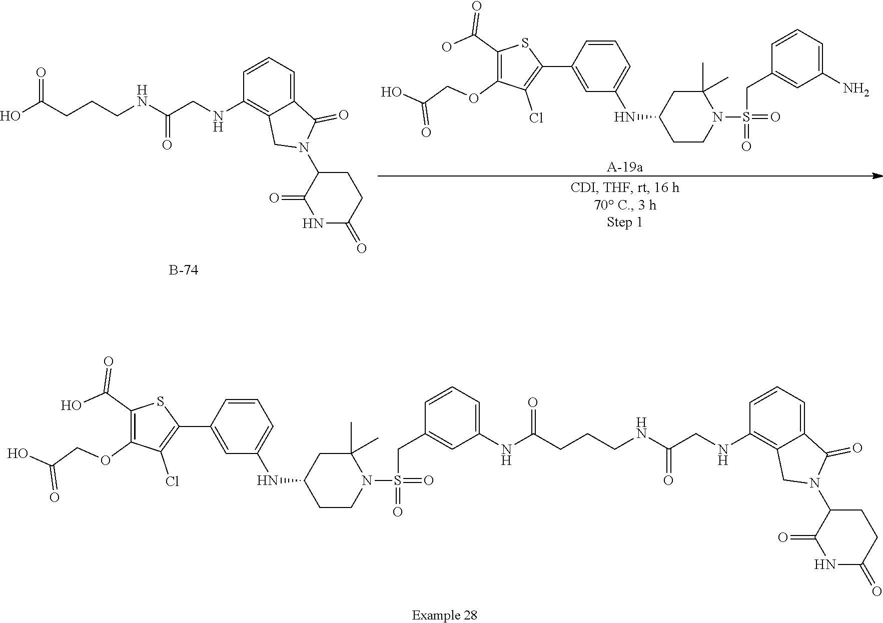

### Step 1: 3-(carboxymethoxy)-4-chloro-5-(3-(((4S)-1-((3-(4-(2-((2-(2,6-dioxopiperidin-3-yl)-1-oxoisoindolin-4-yl)amino)acetamido) butanamido)benzyl)sulfonyl)-2,2-dimethylpiperidin-4-yl)amino)phenyl)thiophene-2-carboxylic acid (28)

3-(carboxymethoxy)-4-chloro-5-(3-(((4S)-1-((3-(4-(2-((2-(2,6-dioxopiperidin-3-yl)-1-oxoisoindolin-4-yl)amino)acetamido) butanamido)benzyl)sulfonyl)-2,2-dimethylpiperidin-4-yl)amino)phenyl)thiophene-2-carboxylic acid (28, 5.43 mg, 5.29 μmol, 16% yield) was synthesized from 4-[[2-[[2-(2,6-dioxo-3-piperidyl)-1-oxo-isoindolin-4-yl]amino]acetyl]amino]butanoic acid (B-74) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except heating to 70° C. was required after amine was added. The residue was purified by reverse phase preparative HPLC [Column: SunFire C18 (19×150 mm) 5 μm, Mobile phase A: 0.1% TFA in water; Mobile phase B: MeCN]. UPLC (ES+): m/z 992.8 [M+H]⁺. ¹H NMR (400 MHz, DMSO-d6): δ 8.05 (t, J=6.00 Hz, 1H), 7.65 (s, 1H), 7.58 (d, J=8.40 Hz, 1H), 7.28 (q, J=7.60 Hz, 1H), 7.19 (t, J=8.00 Hz, 1H), 7.07 (d, J=4.80 Hz, 1H), 6.97 (d, J=7.60 Hz, 1H), 6.86 (s, 1H), 6.80 (d, J=7.60 Hz, 1H), 6.68 (d, J=8.40 Hz, 1H), 6.57 (d, J=8.00 Hz, 1H), 5.12 (dd, J=5.60, 13.00 Hz, 1H), 4.91 (s, 2H), 4.40-4.16 (m, 4H), 3.96-3.41 (m, 7H), 3.16-3.08 (m, 4H), 2.95-2.87 (m, 2H), 2.36-2.28 (m, 3H), 2.00-1.69 (m, 5H), 1.45-1.36 (m, 7H), 1.23-1.12 (m, 1H).

### Example 29

### 3-(carboxymethoxy)-4-chloro-5-[3-[[(4S)-1-[[3-[[2-[2-(2,6-dioxo-3-piperidyl)-1-oxo-isoindolin-4-yl]oxyacetyl]amino]phenyl]methylsulfonyl]-2,2-dimethyl-4-piperidyl]amino]phenyl]thiophene-2-carboxylic acid (29)

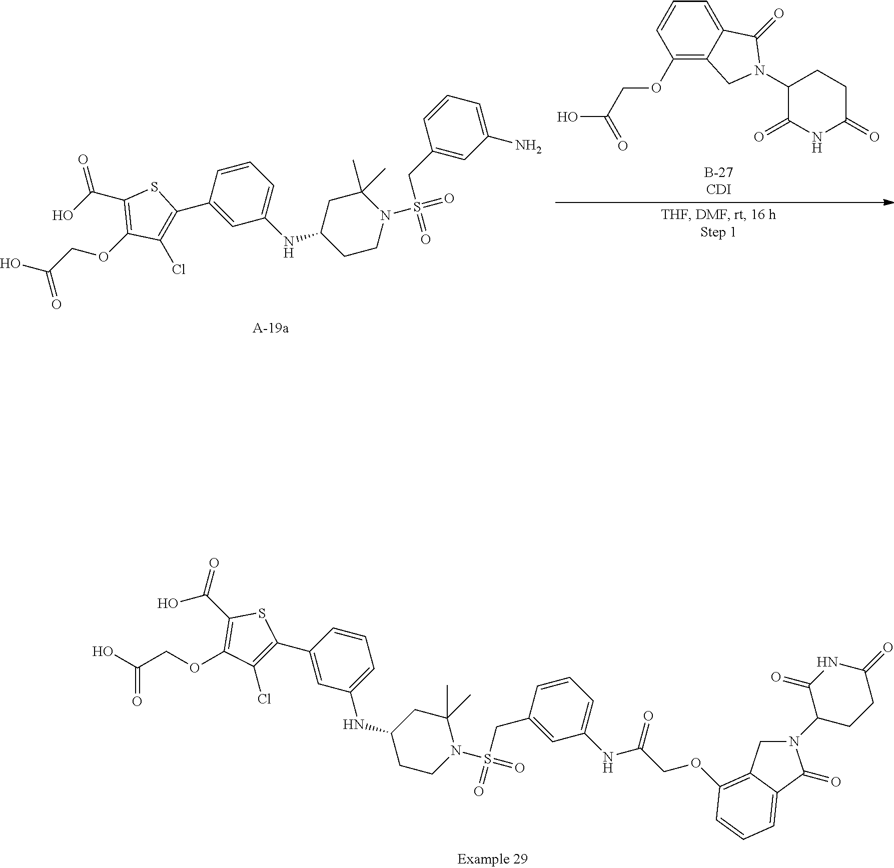 [SMILES: CC1(C)C[C@@H](Nc2cccc(-c3sc(C(=O)O)c(OCC(=O)O)c3Cl)c2)CCN1S(=O)(=O)Cc1cccc(N)c1 ; O=C(O)COc1cccc2c1CN(C1CCC(=O)NC1=O)C2=O ; CC1(C)C[C@@H](Nc2cccc(-c3sc(C(=O)O)c(OCC(=O)O)c3Cl)c2)CCN1S(=O)(=O)Cc1cccc(NC(=O)COc2cccc3c2CN(C2CCC(=O)NC2=O)C3=O)c1]

### Step 1: 3-(carboxymethoxy)-4-chloro-5-[3-[[(4S)-1-[[3-[[2-[2-(2,6-dioxo-3-piperidyl)-1-oxo-isoindolin-4-yl]oxyacetyl]amino]phenyl]methylsulfonyl]-2,2-dimethyl-4-piperidyl]amino]phenyl]thiophene-2-carboxylic acid (29)

3-(carboxymethoxy)-4-chloro-5-[3-[[(4S)-1-[[3-[[2-[2-(2,6-dioxo-3-piperidyl)-1-oxo-isoindolin-4-yl]oxyacetyl]amino]phenyl]methylsulfonyl]-2,2-dimethyl-4-piperidyl]amino]phenyl]thiophene-2-carboxylic acid (29, 25 mg, 24.24 μmol, 15% yield, TFA salt) was synthesized from 2-[2-(2,6-dioxo-3-piperidyl)-1-oxo-isoindolin-4-yl]oxyacetic acid (B-27) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except 1:1 DMF:THF was used as solvent. The residue was purified by reverse phase preparative HPLC [Column: XBridge, C18 (150×19 mm), 5 μm; Mobile phase A: 0.1% TFA in water and Mobile phase B: MeCN]. LCMS (ES+): m/z 908.1 [M+H]⁺. ¹H NMR (400 MHz, DMSO-d₆) δ 10.99 (s, 1H), 10.23 (s, 1H), 7.71 (s, 1H), 7.61 (d, J=8.2 Hz, 1H), 7.46 (t, J=7.8 Hz, 1H), 7.38-7.31 (m, 2H), 7.23-7.11 (m, 3H), 6.88 (s, 1H), 6.80 (d, J=7.6 Hz, 1H), 6.70-6.65 (m, 1H), 5.12 (dd, J=13.3, 5.1 Hz, 1H), 4.91 (s, 2H), 4.89-4.87 (m, 2H), 4.50-4.27 (m, 4H), 3.16-3.06 (m, 2H), 2.91 (ddd, J=17.9, 13.6, 5.3 Hz, 1H), 2.62-2.55 (m, 2H), 2.04-1.95 (m, 1H), 1.92-1.83 (m, 1H), 1.82-1.73 (m, 1H), 1.46 (s, 3H), 1.43-1.34 (m, 4H), 1.29-1.06 (m, 2H).

### Example 30

### 3-(carboxymethoxy)-4-chloro-5-[3-[[1-[[3-[7-[[2-[[2-(2,6-dioxo-3-piperidyl)-3-oxo-isoindolin-4-yl]amino]acetyl]amino]heptanoylamino]phenyl]methylsulfonyl]-4-piperidyl]amino]phenyl]thiophene-2-carboxylic acid (30)

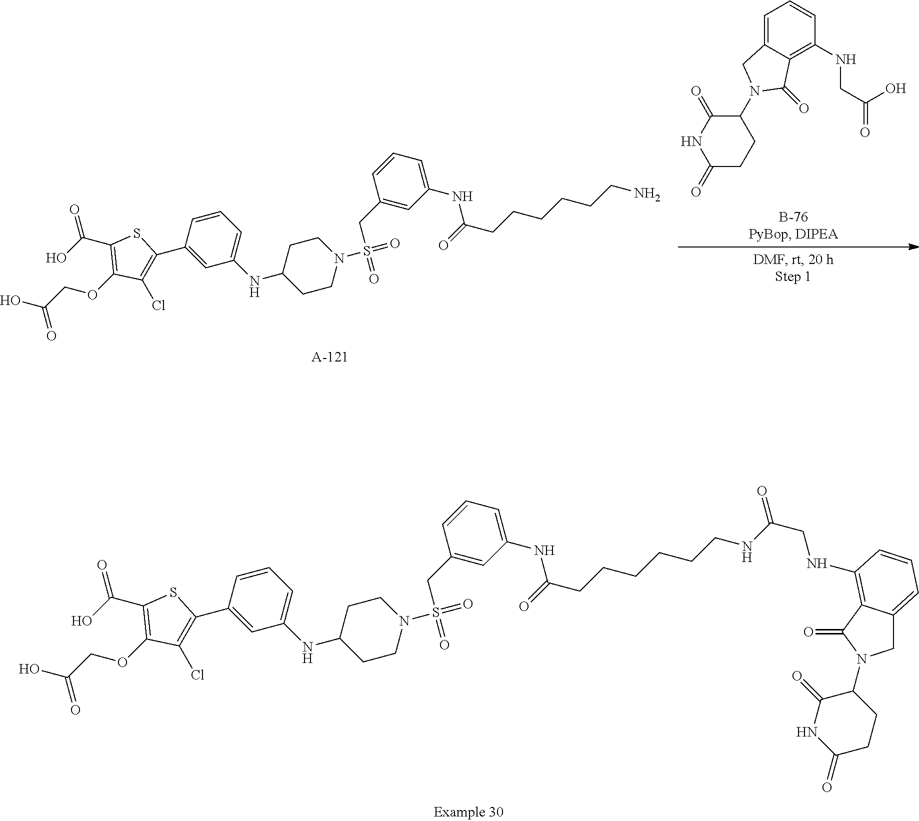 [SMILES: NCCCCCCC(=O)Nc1cccc(CS(=O)(=O)N2CCC(Nc3cccc(-c4sc(C(=O)O)c(OCC(=O)O)c4Cl)c3)CC2)c1 ; O=C(O)CNc1cccc2c1C(=O)N(C1CCC(=O)NC1=O)C2 ; "A-121" ; O=C(O)COc1c(C(=O)O)sc(-c2cccc(NC3CCN(S(=O)(=O)Cc4cccc(NC(=O)CCCCCCNC(=O)CNc5cccc6c5C(=O)N(C5CCC(=O)NC5=O)C6)c4)CC3)c2)c1Cl]

### Step 1: 3-(carboxymethoxy)-4-chloro-5-[3-[[1-[[3-[7-[[2-[[2-(2,6-dioxo-3-piperidyl)-3-oxo-isoindolin-4-yl]amino]acetyl]amino]heptanoylamino]phenyl]methylsulfonyl]-4-piperidyl]amino]phenyl]thiophene-2-carboxylic acid (30)

3-(Carboxymethoxy)-4-chloro-5-[3-[[1-[[3-[7-[[2-[[2-(2,6-dioxo-3-piperidyl)-3-oxo-isoindolin-4-yl]amino]acetyl]amino]heptanoylamino]phenyl]methylsulfonyl]-4-piperidyl]amino]phenyl]thiophene-2-carboxylic acid (30, 23 mg, 21.16 μmol, 12% yield, TFA salt) was synthesized from 2-[[2-(2,6-dioxo-3-piperidyl)-3-oxo-isoindolin-4-yl]amino]acetic acid (B-76) and 5-(3-((1-((3-(7-aminoheptanamido)benzyl)sulfonyl)piperidin-4-yl)amino)phenyl)-3-(carboxymethoxy)-4-chlorothiophene-2-carboxylic acid hydrochloride (A-121) in a similar fashion to Compound C-21. The residue was purified by reverse phase preparative HPLC [Purification method: Column: SunFire prep OBD 19×50 mm (5 μm), Mobile phase A: 0.1% TFA in water and Mobile phase B: MeCN]. UPLC (ES+): m/z 1006.9 [M+H]⁺. ¹H NMR (400 MHz, DMSO-d₆) δ 10.97 (s, 1H), 9.95 (s, 1H), 8.00 (t, J=5.7 Hz, 1H), 7.67 (s, 1H), 7.56 (d, J=8.3 Hz, 1H), 7.38-7.25 (m, 2H), 7.20 (t, J=7.9 Hz, 1H), 7.07 (d, J=7.6 Hz, 1H), 6.87 (s, 1H), 6.81 (d, J=7.6 Hz, 1H), 6.70 (d, J=7.4 Hz, 2H), 6.39 (d, J=8.3 Hz, 1H), 5.89 (s, 1H), 5.01 (dd, J=13.3, 5.0 Hz, 1H), 4.92 (s, 2H), 4.39-4.28 (m, 3H), 4.19 (d, J=17.2 Hz, 1H), 3.80 (s, 2H), 3.54 (d, J=12.2 Hz, 2H), 3.08 (q, J=6.6 Hz, 2H), 3.01-2.83 (m, 3H), 2.65-2.58 (m, 1H), 2.43-2.34 (m, 1H), 2.32-2.25 (m, 2H), 2.02-1.88 (m, 3H), 1.62-1.48 (m, 2H), 1.47-1.35 (m, 3H), 1.33-1.22 (m, 4H).

### Example 31

### 3-(carboxymethoxy)-4-chloro-5-[3-[[1-[[3-[6-[4-[[2-(2,6-dioxo-3-piperidyl)-1,3-dioxo-isoindolin-4-yl]amino]-1-piperidyl]hexanoylamino]phenyl]methylsulfonyl]-4-piperidyl]amino]phenyl]thiophene-2-carboxylic acid (31)

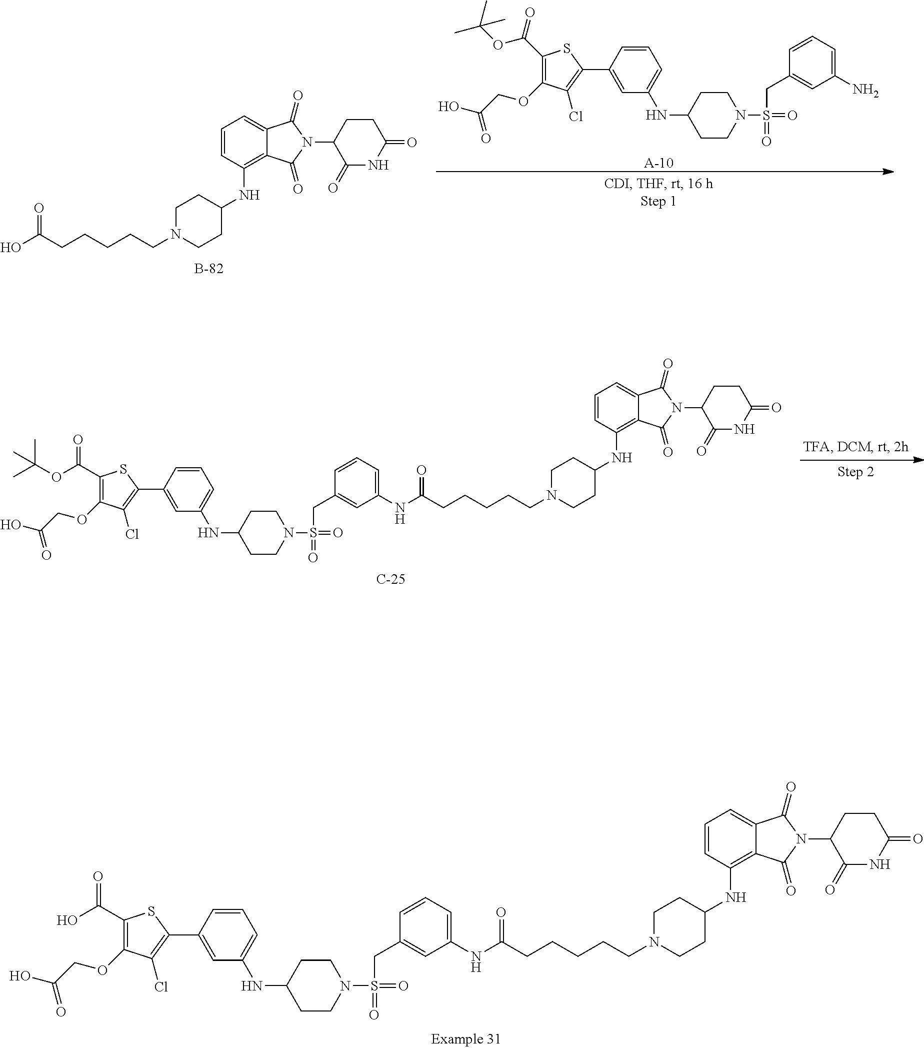

### Step 1: 2-[[2-tert-butoxycarbonyl-4-chloro-5-[3-[[1-[[3-[6-[4-[[2-(2,6-dioxo-3-piperidyl)-1,3-dioxo-isoindolin-4-yl]amino]-1-piperidyl]hexanoylamino]phenyl]methylsulfonyl]-4-piperidyl]amino]phenyl]-3-thienyl]oxy]acetic acid (C-25)

2-[[2-tert-butoxycarbonyl-4-chloro-5-[3-[[1-[[3-[6-[4-[[2-(2,6-dioxo-3-piperidyl)-1,3-dioxo-isoindolin-4-yl]amino]-1-piperidyl]hexanoylamino]phenyl]methylsulfonyl]-4-piperidyl]amino]phenyl]-3-thienyl]oxy]acetic acid (C-25, 22.69 mg, 13.22 μmol, 16% yield, 66% purity, formate salt) was synthesized from 6-[4-[[2-(2,6-dioxo-3-piperidyl)-1,3-dioxo-isoindolin-4-yl]amino]-1-piperidyl]hexanoic acid (B-82) and 2-((5-(3-((1-((3-aminobenzyl)sulfonyl)piperidin-4-yl)amino)phenyl)-2-(tert-butoxycarbonyl)-4-chlorothiophen-3-yl))oxy)acetic acid (A-10) in a similar fashion to Example 23. The residue was purified by reverse phase prep HPLC [Column: SunFire C18 (19×150 mm) 5 μm; Mobile phase A: 0.1% formic acid in water:MeCN and Mobile phase B: MeCN; Flow Rate: 15.0 mL/min]. LCMS (ES+): m/z 1089.3 [M+H]⁺.

### Step 2: 3-(carboxymethoxy)-4-chloro-5-[3-[[1-[[3-[6-[4-[[2-(2,6-dioxo-3-piperidyl)-1,3-dioxo-isoindolin-4-yl]amino]-1-piperidyl]hexanoylamino]phenyl]methylsulfonyl]-4-piperidyl]amino]phenyl]thiophene-2-carboxylic acid (31)

3-(carboxymethoxy)-4-chloro-5-[3-[[1-[[3-[6-[4-[[2-(2,6-dioxo-3-piperidyl)-1,3-dioxo-isoindolin-4-yl]amino]-1-piperidyl]hexanoylamino]phenyl]methylsulfonyl]-4-piperidyl]amino]phenyl]thiophene-2-carboxylic acid (31, 8.18 mg, 7.03 μmol, 53% yield, TFA salt) was synthesized from 2-[[2-tert-butoxycarbonyl-4-chloro-5-[3-[[1-[[3-[6-[4-[[2-(2,6-dioxo-3-piperidyl)-1,3-dioxo-isoindolin-4-yl]amino]-1-piperidyl]hexanoylamino]phenyl]methylsulfonyl]-4-piperidyl]amino]phenyl]-3-thienyl]oxy]acetic acid (C-25) in a similar fashion to Compound C-1, except using 10 eq. of TEA. LCMS (ES+): m/z 1031.8 [M+H]⁺. ¹H NMR (400 MHz, DMSO-d6): δ 11.11 (s, 1H), 10.01 (s, 1H), 9.25 (s, 1H), 7.70-7.58 (m, 3H), 7.31-7.20 (m, 3H), 7.12-7.08 (m, 2H), 6.89-6.70 (m, 3H), 6.30-6.25 (m, 1H), 5.15-5.00 (m, 1H), 4.92 (s, 2H), 4.37 (s, 2H), 3.42-3.40 (m, 2H), 3.17-2.90 (m, 10H), 2.74-2.68 (m, 2H), 2.36-2.34 (m, 3H), 2.20-1.94 (m, 4H), 1.70-1.50 (m, 6H), 1.28-1.15 (m, 10H).

### Example 32

### 3-(carboxymethoxy)-4-chloro-5-[3-[[(4S)-1-[[3-[[1-[2-[[2-(2,6-dioxo-3-piperidyl)-1,3-dioxo-isoindolin-4-yl]amino]acetyl]piperidine-4-carbonyl]amino]phenyl]methylsulfonyl]-2,2-dimethyl-4-piperidyl]amino]phenyl]thiophene-2-carboxylic acid (32)

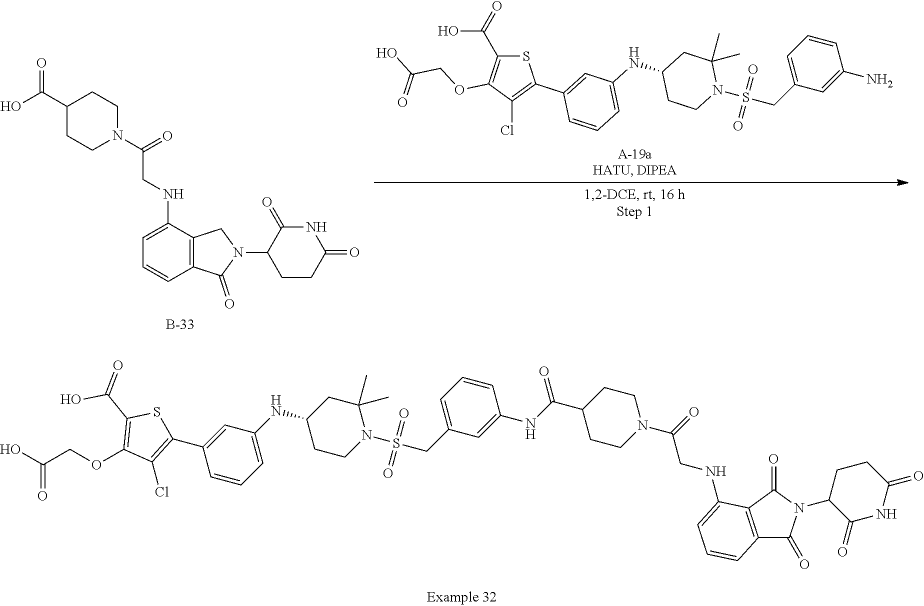

### Step 1: 3-(carboxymethoxy)-4-chloro-5-[3-[[(4S)-1-[[3-[[1-[2-[[2-(2,6-dioxo-3-piperidyl)-1,3-dioxo-isoindolin-4-yl]amino]acetyl]piperidine-4-carbonyl]amino]phenyl]methylsulfonyl]-2,2-dimethyl-4-piperidyl]amino]phenyl]thiophene-2-carboxylic acid (32)

3-(carboxymethoxy)-4-chloro-5-[3-[[(4S)-1-[[3-[[1-[2-[[2-(2,6-dioxo-3-piperidyl)-1,3-dioxo-isoindolin-4-yl]amino]acetyl]piperidine-4-carbonyl]amino]phenyl]methylsulfonyl]-2,2-dimethyl-4-piperidyl]amino]phenyl]thiophene-2-carboxylic acid (32, 7.50 mg, 6.97 μmol, 8% yield) was synthesized from 1-[2-[[2-(2,6-dioxo-3-piperidyl)-1,3-dioxo-isoindolin-4-yl]amino]acetyl]piperidine-4-carboxylic acid (B-33) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1. The residue was purified by reverse phase prep HPLC (Column: Zorbax diphenyl; Mobile phase: 0.1% TFA in Water/MeCN). LCMS (ES+): m/z 1032.0 [M+H]⁺. ¹H NMR (400 MHz, DMSO-d6): δ 11.12 (s, 1H), 10.04 (s, 1H), 7.70 (s, 1H), 7.60-7.61 (m, 2H), 7.31 (t, J=7.96 Hz, 1H), 7.20 (t, J=8.04 Hz, 1H), 7.07-7.09 (m, 4H), 6.86 (s, 1H), 6.80 (d, J=7.44 Hz, 1H), 6.69 (d, J=8.08 Hz, 1H), 6.55 (s, 2H), 5.69 (d, J=5.72 Hz, 1H), 5.06-5.07 (m, 1H), 4.89 (s, 2H), 4.39-4.42 (m, 2H), 4.15-4.19 (m, 3H), 3.96-3.99 (m, 1H), 3.34-3.52 (m, 2H), 3.08-3.11 (m, 2H), 2.72-2.75 (m, 3H), 2.03-2.04 (m, 2H), 1.67-1.70 (m, 5H), 1.40-1.48 (m, 8H), 1.12-1.15 (m, 1H).

### Example 33

### 3-(carboxymethoxy)-4-chloro-5-[3-[[1-[[3-[6-[4-[[2-(2,6-dioxo-3-piperidyl)-1-oxo-isoindolin-4-yl]amino]-1-piperidyl]hexanoylamino]phenyl]methylsulfonyl]-4-piperidyl]amino]phenyl]thiophene-2-carboxylic acid (33)

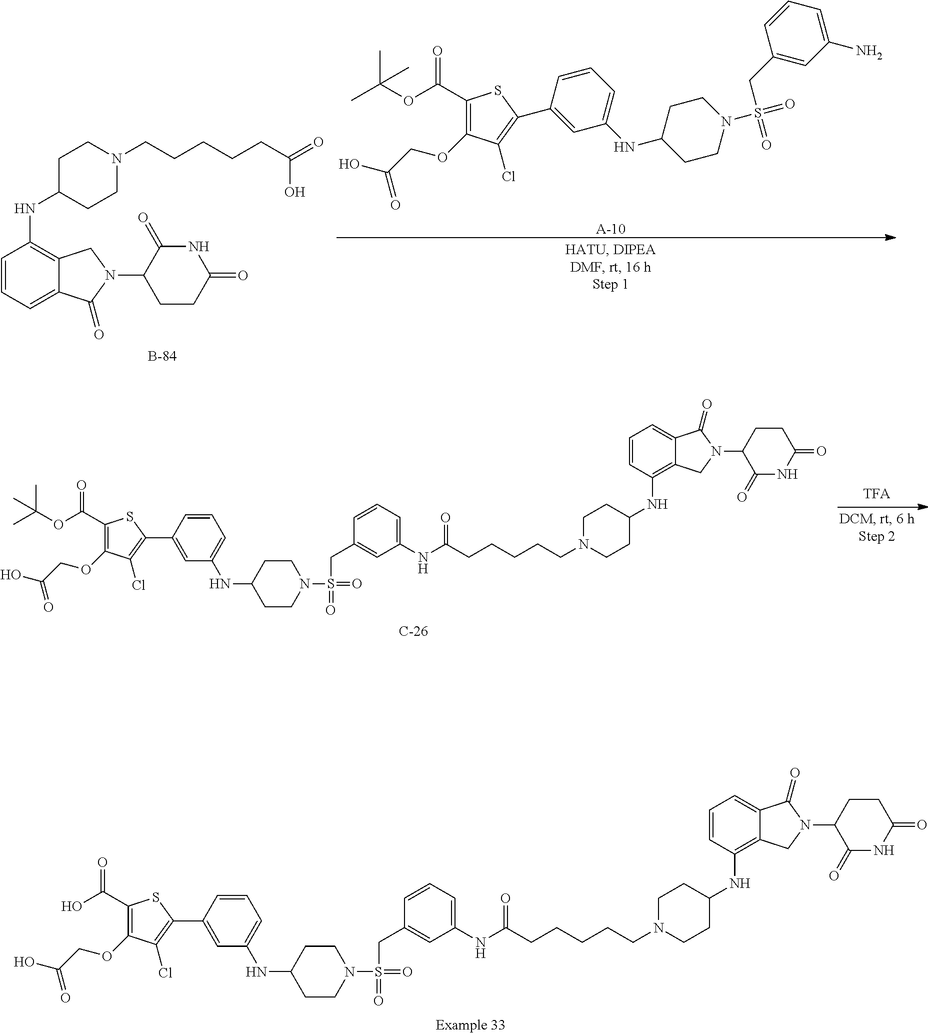

### Step 1: 2-[[2-tert-butoxycarbonyl-4-chloro-5-[3-[[1-[[3-[6-[4-[[2-(2,6-dioxo-3-piperidyl)-1-oxo-isoindolin-4-yl]amino]-1-piperidyl]hexanoylamino]phenyl]methylsulfonyl]-4-piperidyl]amino]phenyl]-3-thienyl]oxy]acetic acid (C-26)

2-[[2-tert-butoxycarbonyl-4-chloro-5-[3-[[1-[[3-[6-[4-[[2-(2,6-dioxo-3-piperidyl)-1-oxo-isoindolin-4-yl]amino]-1-piperidyl]hexanoylamino]phenyl]methylsulfonyl]-4-piperidyl]amino]phenyl]-3-thienyl]oxy]acetic acid (C-26, 32 mg, 22.03 μmol, 13% yield, TFA salt) was synthesized from 6-[4-[[2-(2,6-dioxo-3-piperidyl)-1-oxo-isoindolin-4-yl]amino]-1-piperidyl]hexanoic acid (B-84) and 2-[[5-[3-[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The residue was purified by reverse phase prep HPLC [Purification method: Column: SunFire-C18 100 mm, Mobile phase A: 0.1% TFA in water, Mobile phase B: MeCN; Flow Rate: 15.0 mL/min]. LCMS (ES+): m/z 1074.9 [M+H]⁺.

### Step 2: 3-(carboxymethoxy)-4-chloro-5-(3-((1-((3-(6-(4-((2-(2,6-dioxopiperidin-3-yl)-1-oxoisoindolin-4-yl)amino)piperidin-1-yl) hexanamido)benzyl)sulfonyl)piperidin-4-yl)amino)phenyl)thiophene-2-carboxylic acid (33)

3-(carboxymethoxy)-4-chloro-5-[3-[[1-[[3-[6-[4-[[2-(2,6-dioxo-3-piperidyl)-1-oxo-isoindolin-4-yl]amino]-1-piperidyl]hexanoylamino]phenyl]methylsulfonyl]-4-piperidyl]amino]phenyl]thiophene-2-carboxylic acid (33, 14.24 mg, 13.84 mol, 74% yield, TFA salt) was synthesized from 2-[[2-tert-butoxy carbonyl-4-chloro-5-[3-[[1-[[3-[6-[4-[[2-(2,6-dioxo-3-piperidyl)-1-oxo-isoindolin-4-yl]amino]-1-piperidyl]hexanoylamino]phenyl]methylsulfonyl]-4-piperidyl]amino]phenyl]-3-thienyl]oxy]acetic acid (C-26) in a similar fashion to Compound C-1, except using 5 eq. of TFA. The residue was purified by reverse phase prep HPLC [Purification method: Column: SunFire-C18 100 mm, Mobile phase A: 0.1% TFA in water; Mobile phase B: MeCN; Flow Rate: 15.0 mL/min]. LCMS: UPLC (ES+): m/z 1018.2 [M+H]⁺. ¹H NMR (400 MHz, DMSO-d6): δ 13.00 (s, 1H), 11.03 (s, 1H), 10.01 (s, 1H), 9.25 (s, 1H), 7.68 (s, 1H), 7.59 (d, J=7.88 Hz, 1H), 7.33-7.29 (m, 2H), 7.22 (t, J=7.76 Hz, 1H), 7.09 (d, J=7.52 Hz, 1H), 7.02-6.97 (m, 1H), 6.89-6.82 (m, 3H), 6.72 (d, J=8.00 Hz, 1H), 5.60 (s, 1H), 5.16-5.12 (m, 1H), 4.93 (S, 2H), 4.37 (s, 2H), 4.26-4.12 (m, 2H), 3.48-3.43 (m, 4H), 3.18 (s, 2H), 3.06-2.91 (m, 7H), 2.34-2.27 (m, 3H), 2.15-1.95 (m, 6H), 1.66-1.63 (m, 6H), 1.42-1.35 (m, 4H).

### Example 34

### 3-(carboxymethoxy)-4-chloro-5-[3-[[(4S)-1-[[3-[[2-[[2-(2,6-dioxo-3-piperidyl)-1,3-dioxo-isoindolin-5-yl]amino]acetyl]amino]phenyl]methylsulfonyl]-2,2-dimethyl-4-piperidyl]amino]phenyl]thiophene-2-carboxylic acid (34)

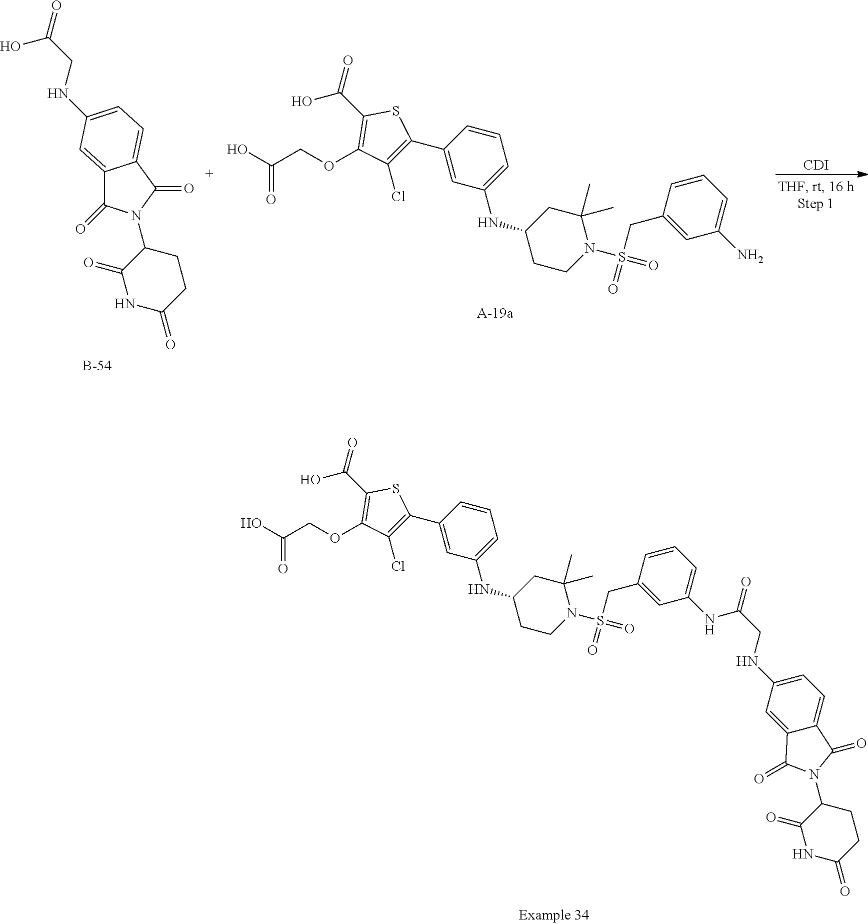

### Step 1: 3-(carboxymethoxy)-4-chloro-5-[3-[[(4S)-1-[[3-[[2-[[2-(2,6-dioxo-3-piperidyl)-1,3-dioxo-isoindolin-5-yl]amino]acetyl]amino]phenyl]methylsulfonyl]-2,2-dimethyl-4-piperidyl]amino]phenyl]thiophene-2-carboxylic acid (34)

3-(carboxymethoxy)-4-chloro-5-[3-[[(4S)-1-[[3-[[2-[[2-(2,6-dioxo-3-piperidyl)-1,3-dioxo-isoindolin-5-yl]amino]acetyl]amino]phenyl]methylsulfonyl]-2,2-dimethyl-4-piperidyl]amino]phenyl]thiophene-2-carboxylic acid (34, 10.42 mg, 10.90 μmol, 12% yield, TFA salt) was synthesized from 2-[[2-(2,6-dioxo-3-piperidyl)-1,3-dioxo-isoindolin-5-yl]amino]acetic acid (B-54) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23. The residue was purified by reverse phase prep HPLC [Purification method: Column: SunFire C18 (19×150 mm) 5 μm; Mobile phase A: 0.1% TFA in water/MeCN; Mobile phase B: MeCN]. LCMS (ES+): m/z 920.8 [M+H]⁺. ¹H NMR (400 MHz, DMSO-d₆) δ 11.06 (s, 1H), 10.24 (s, 1H), 7.68 (s, 1H), 7.65-7.56 (m, 2H), 7.46-7.39 (m, 1H), 7.33 (t, J=7.9 Hz, 1H), 7.22-7.15 (m, 1H), 7.11 (d, J=7.6 Hz, 1H), 7.02 (s, 1H), 6.92 (d, J=8.4 Hz, 1H), 6.86 (s, 1H), 6.79 (d, J=7.6 Hz, 1H), 6.67 (d, J=8.3 Hz, 1H), 5.70 (s, 1H), 5.03 (dd, J=12.9, 5.3 Hz, 1H), 4.91 (d, J=1.8 Hz, 2H), 4.42 (d, J=13.7 Hz, 1H), 4.30 (d, J=13.7 Hz, 1H), 4.11 (d, J=5.5 Hz, 2H), 3.57-3.41 (m, 3H), 3.12 (t, J=12.6 Hz, 1H), 2.93-2.80 (m, 1H), 2.12-2.05 (m, 1H), 2.04-1.95 (m, 1H), 1.92-1.83 (m, 1H), 1.82-1.74 (m, 1H), 1.46 (s, 3H), 1.43-1.34 (m, 4H), 1.18-1.03 (m, 1H).

### Example 35

### 3-(carboxymethoxy)-4-chloro-5-[3-[[(4S)-1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-2,2-dimethyl-4-piperidyl]amino]phenyl]thiophene-2-carboxylic acid (35)

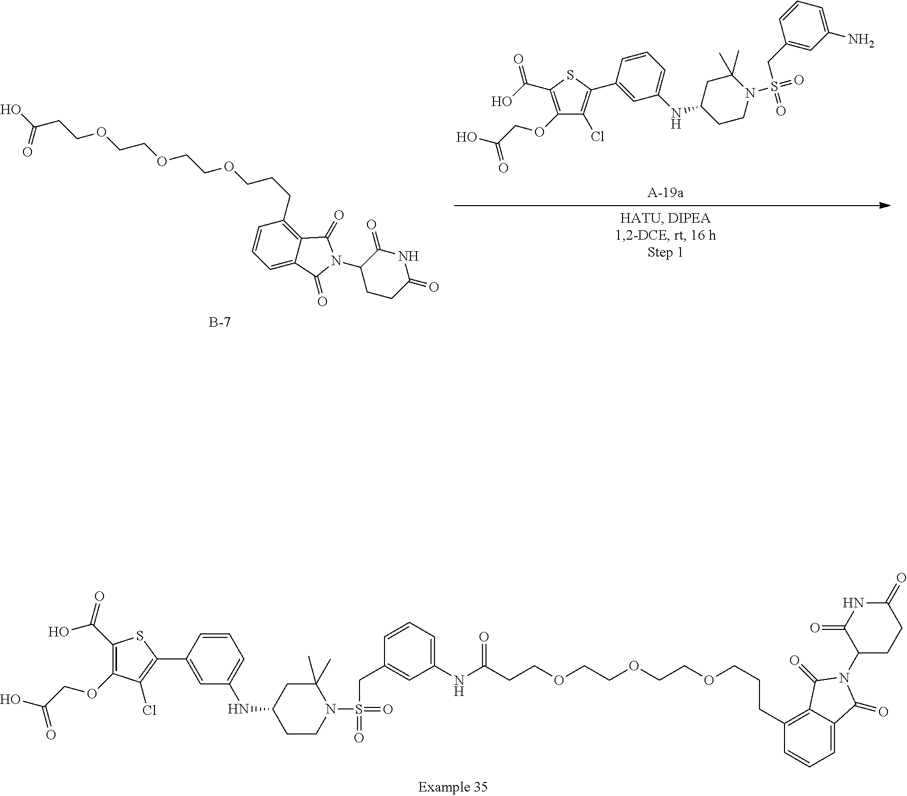

### Step 1: 3-(carboxymethoxy)-4-chloro-5-[3-[[(4S)-1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-2,2-dimethyl-4-piperidyl]amino]phenyl]thiophene-2-carboxylic acid (35)

3-(carboxymethoxy)-4-chloro-5-[3-[[(4S)-1-[[3-[3-[2-[2-[3-[2-(2,6-dioxo-3-piperidyl)-1,3-dioxo-isoindolin-4-yl]propoxy]ethoxy]ethoxy]propanoylamino]phenyl]methylsulfonyl]-2,2-dimethyl-4-piperidyl]amino]phenyl]thiophene-2-carboxylic acid (35, 19.50 mg, 17.81 μmol, 14% yield, TFA salt) was synthesized from 3-[2-[2-[3-[2-(2,6-dioxo-3-piperidyl)-1,3-dioxo-isoindolin-4-yl]propoxy]ethoxy]ethoxy]propanoic acid (B-7) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1. The residue was purified by reverse phase preparative HPLC [Column: SunFire C18 (19×150 mm) 5 μm; Mobile phase A: 0.1% TFA in water and Mobile phase B: MeCN]. LCMS (ES+): m/z=1066.1 [M+H]⁺. ¹H-NMR (400 MHz, DMSO-d6): δ 11.13 (s, 1H), 10.01 (s, 1H), 7.76-7.69 (m, 4H), 7.62-7.58 (m, 1H), 7.29 (t, J=7.80 Hz, 1H), 7.20 (d, J=8.36 Hz, 1H), 7.07 (d, J=7.76 Hz, 1H), 6.88 (s, 1H), 6.81 (d, J=6.84 Hz, 1H), 6.70 (d, J=8.52 Hz, 1H), 5.13 (dd, J=5.12, 12.78 Hz, 1H), 4.92 (s, 1H), 4.41-4.25 (m, 2H), 3.51-3.45 (m, 13H), 3.07-3.05 (m, 3H), 2.10-2.01 (m, 2H), 1.88-1.77 (m, 5H), 1.49-1.39 (m, 7H), 1.26-1.10 (m, 2H).

### Example 36

### 3-(carboxymethoxy)-4-chloro-5-[3-[[1-[[3-[[2-[[2-(2,6-dioxo-3-piperidyl)-1-oxo-isoindolin-5-yl]amino]acetyl]amino]phenyl]methylsulfonyl]-4-piperidyl]amino]phenyl]thiophene-2-carboxylic acid (36)

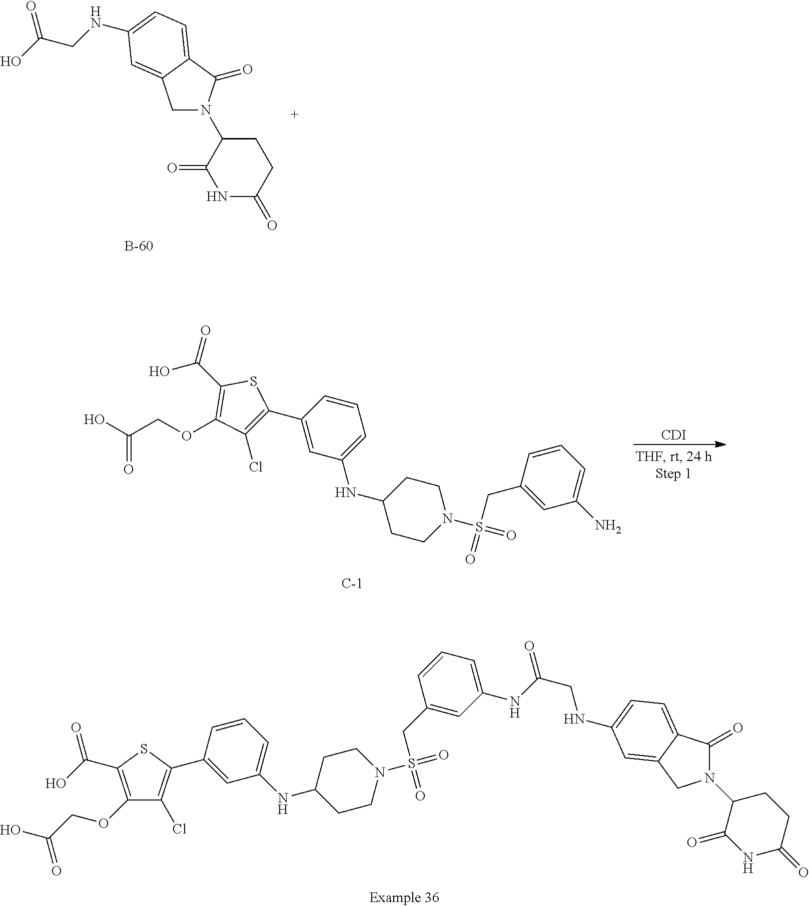

### Step 1: 3-(carboxymethoxy)-4-chloro-5-[3-[[1-[[3-[[2-[[2-(2,6-dioxo-3-piperidyl)-1-oxo-isoindolin-5-yl]amino]acetyl]amino]phenyl]methylsulfonyl]-4-piperidyl]amino]phenyl]thiophene-2-carboxylic acid (36)

3-(carboxymethoxy)-4-chloro-5-[3-[[1-[[3-[[2-[[2-(2,6-dioxo-3-piperidyl)-1-oxo-isoindolin-5-yl]amino]acetyl]amino]phenyl]methylsulfonyl]-4-piperidyl]amino]phenyl]thiophene-2-carboxylic acid (36, 8.16 mg, 7.82 μmol, 6% yield, TFA salt) was synthesized from 2-[[2-(2,6-dioxo-3-piperidyl)-1-oxo-isoindolin-5-yl]amino]acetic acid (B-60) and 5-[3-[[1-[(3-aminophenyl)methylsulfonyl]-4-piperidyl]amino]phenyl]-3-(carboxymethoxy)-4-chloro-thiophene-2-carboxylic acid (C-1) in a similar fashion to Example 23, except using DMF as the solvent. The residue was purified by reverse phase prep HPLC [Column: Sun fire C18 (19×150 mm) 5.0 μm, Mobile phase A: 0.1% TFA in water and mobile phase B: MeCN]. LCMS (ES+): m/z 878.8 [M+H]⁺. ¹H NMR (400 MHz, DMSO-d₆) δ 10.92 (s, 1H), 10.15 (s, 1H), 7.66 (s, 1H), 7.62 (d, J=8.5 Hz, 1H), 7.43 (d, J=8.4 Hz, 1H), 7.35-7.29 (m, 1H), 7.24-7.16 (m, 1H), 7.10 (d, J=8.1 Hz, 1H), 6.87 (s, 1H), 6.82 (d, J=7.6 Hz, 1H), 6.75-6.65 (m, 4H), 5.01 (dd, J=13.2, 5.1 Hz, 1H), 4.92 (s, 3H), 4.36 (s, 2H), 4.27 (d, J=16.9 Hz, 1H), 4.13 (d, J=16.8 Hz, 1H), 3.98 (s, 2H), 3.58-3.51 (m, 12H), 3.01-2.81 (m, 4H), 2.62-2.55 (m, 1H), 2.38-2.23 (m, 1H), 2.00-1.86 (m, 4H), 1.44-1.30 (m, 3H).

### Example 37

### 3-(carboxymethoxy)-4-chloro-5-[3-[[(4S)-1-[[3-[[2-[[2-(2,6-dioxo-3-piperidyl)-1-oxo-isoindolin-5-yl]amino]acetyl]amino]phenyl]methylsulfonyl]-2,2-dimethyl-4-piperidyl]amino]phenyl]thiophene-2-carboxylic acid (37)

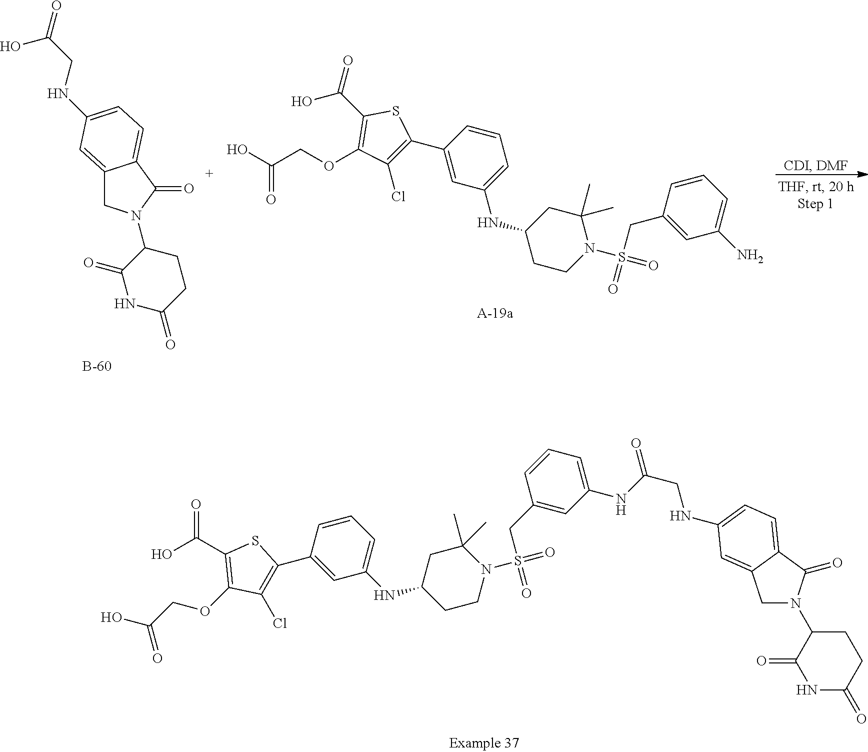

### Step 1: 3-(carboxymethoxy)-4-chloro-5-[3-[[(4S)-1-[[3-[[2-[[2-(2,6-dioxo-3-piperidyl)-1-oxo-isoindolin-5-yl]amino]acetyl]amino]phenyl]methylsulfonyl]-2,2-dimethyl-4-piperidyl]amino]phenyl]-thiophene-2-carboxylic acid (37)

3-(carboxymethoxy)-4-chloro-5-[3-[[(4S)-1-[[3-[[2-[[2-(2,6-dioxo-3-piperidyl)-1-oxo-isoindolin-5-yl]amino]acetyl]amino]phenyl]methylsulfonyl]-2,2-dimethyl-4-piperidyl]amino]phenyl]-thiophene-2-carboxylic acid (37, 13 mg, 14.22 μmol, 8% yield) was synthesized from 2-[[2-(2,6-dioxo-3-piperidyl)-1-oxo-isoindolin-5-yl]amino]acetic acid (B-60) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except using DMF as the solvent. The residue was purified by reverse phase prep HPLC [Column: Sun fire C18 (19×150 mm) 5.0 μm, Mobile phase A: 0.1% TFA in water and mobile phase B: MeCN]. LCMS (ES+): m/z 907.1 [M+H]⁺. ¹H NMR (400 MHz, DMSO-d₆) δ 10.91 (s, 1H), 10.13 (s, 1H), 7.69 (s, 1H), 7.62 (d, J=8.2 Hz, 1H), 7.42 (d, J=8.4 Hz, 1H), 7.32 (t, J=7.9 Hz, 1H), 7.19 (t, J=7.9 Hz, 1H), 7.10 (d, J=7.6 Hz, 1H), 6.88 (s, 1H), 6.80 (d, J=7.6 Hz, 1H), 6.76-6.71 (m, 1H), 6.71-6.65 (m, 2H), 5.00 (dd, J=13.3, 5.1 Hz, 1H), 4.91 (s, 2H), 4.41 (d, J=13.8 Hz, 1H), 4.34-4.21 (m, 2H), 4.12 (d, J=16.8 Hz, 1H), 3.99 (s, 2H), 3.57-3.48 (m, 1H), 3.48-3.39 (m, 1H), 3.11 (t, J=12.6 Hz, 1H), 2.95-2.81 (m, 1H), 2.63-2.54 (m, 1H), 2.32-2.23 (m, 1H), 1.98-1.83 (m, 2H), 1.82-1.73 (m, 1H), 1.46 (s, 3H), 1.43-1.35 (m, 4H), 1.18-1.04 (m, 1H).

### Example 38

### 3-(carboxymethoxy)-4-chloro-5-[3-[[1-[[3-[3-[4-[[[2-(2,6-dioxo-3-piperidyl)-1-oxo-isoindolin-4-yl]amino]methyl]pyrazol-1-yl]propanoylamino]phenyl]methylsulfonyl]-4-piperidyl]amino]phenyl]thiophene-2-carboxylic acid (38)

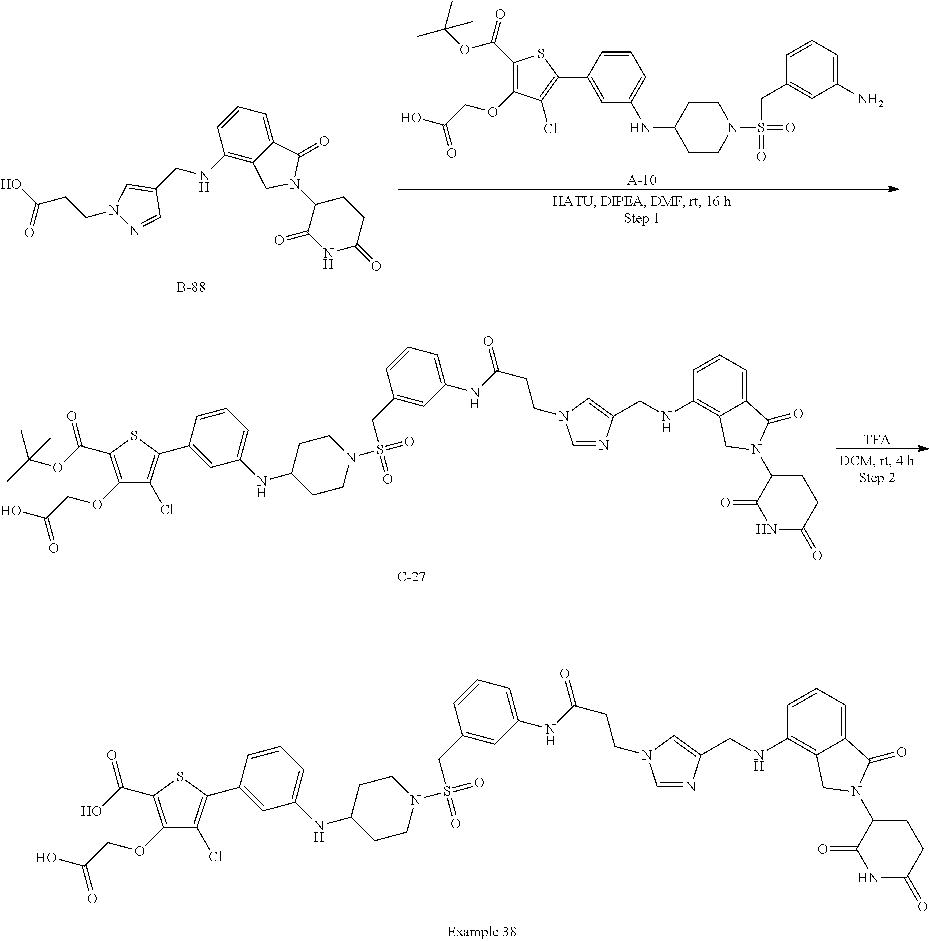

### Step 1: 2-[[2-tert-butoxycarbonyl-4-chloro-5-[3-[[1-[[3-[3-[4-[[[2-(2,6-dioxo-3-piperidyl)-1-oxo-isoindolin-4-yl]amino]methyl]pyrazol-1-yl]propanoylamino]phenyl]methylsulfonyl]-4-piperidyl]amino]phenyl]-3-thienyl]oxy]acetic acid (C-27)

2-[[2-tert-butoxycarbonyl-4-chloro-5-[3-[[1-[[3-[3-[4-[[[2-(2,6-dioxo-3-piperidyl)-1-oxo-isoindolin-4-yl]amino]methyl]pyrazol-1-yl]propanoylamino]phenyl]methylsulfonyl]-4-piperidyl]amino]phenyl]-3-thienyl]oxy]acetic acid (C-27, 100 mg) was synthesized from 3-[4-[[[2-(2,6-dioxo-3-piperidyl)-1-oxo-isoindolin-4-yl]amino]methyl]pyrazol-1-yl]propanoic acid (B-8 and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. UPLC-MS (ES+): m/z 1029.8 [M+H]⁺.

### Step 2: 3-(carboxymethoxy)-4-chloro-5-[3-[[1-[[3-[3-[4-[[[2-(2,6-dioxo-3-piperidyl)-1-oxo-isoindolin-4-yl]amino]methyl]pyrazol-1-yl]propanoylamino]phenyl]methylsulfonyl]-4-piperidyl]amino]phenyl]thiophene-2-carboxylic acid (38)

3-(carboxymethoxy)-4-chloro-5-[3-[[1-[[3-[3-[4-[[[2-(2,6-dioxo-3-piperidyl)-1-oxo-isoindolin-4-yl]amino]methyl]pyrazol-1-yl]propanoylamino]phenyl]methylsulfonyl]-4-piperidyl]amino]phenyl]thiophene-2-carboxylic acid (38, 4.2 mg, 4.21 μmol, 2% yield, TFA salt) was synthesized from 2-[2-tert-butoxycarbonyl-4-chloro-5-[3-[[1-[[3-[3-[4-[[[2-(2,6-dioxo-3-piperidyl)-1-oxo-isoindolin-4-yl]amino]methyl]pyrazol-1-yl]propanoylamino]phenyl]methylsulfonyl]-4-piperidyl]amino]phenyl]-3-thienyl]oxy]acetic acid (C-27) in a similar fashion to Compound C-1. The residue was purified by reverse phase prep HPLC [Purification method: Column: XSELECT-C18 (150×19) mm, 5 μm; Mobile phase A: 0.1% TFA in water and Mobile phase B: MeCN]. LCMS (ES+): m/z 973.0 [M+H]⁺. ¹H NMR (400 MHz, DMSO-d6): δ 11.01 (s, 1H), 10.09 (s, 1H), 7.78 (s, 1H), 7.65-7.63 (m, 2H), 7.56 (d, J=8.32 Hz, 1H), 7.42 (s, 1H), 7.31-7.29 (m, 1H), 7.23-7.19 (m, 2H), 7.09 (d, J=7.84 Hz, 1H), 6.93 (d, J=7.20 Hz, 1H), 6.88 (s, 1H), 6.83-6.77 (m, 4H), 5.91-5.80 (m, 1H), 5.12-5.09 (m, 1H), 4.92 (s, 2H), 4.36-4.15 (m, 8H), 3.57-3.51 (m, 2H), 3.17-0.87 (m, 6H), 2.05-1.94 (m, 2H), 1.39-1.24 (m, 2H).

### Example 39

### 3-(carboxymethoxy)-4-chloro-5-[3-[[1-[1-[3-[7-[[2-[[2-(2,6-dioxo-3-piperidyl)-1-oxo-isoindolin-5-yl]amino]acetyl]amino]heptanoylamino]phenyl]-1-methyl-ethyl]sulfonyl-4-piperidyl]amino]phenyl]thiophene-2-carboxylic acid (39)

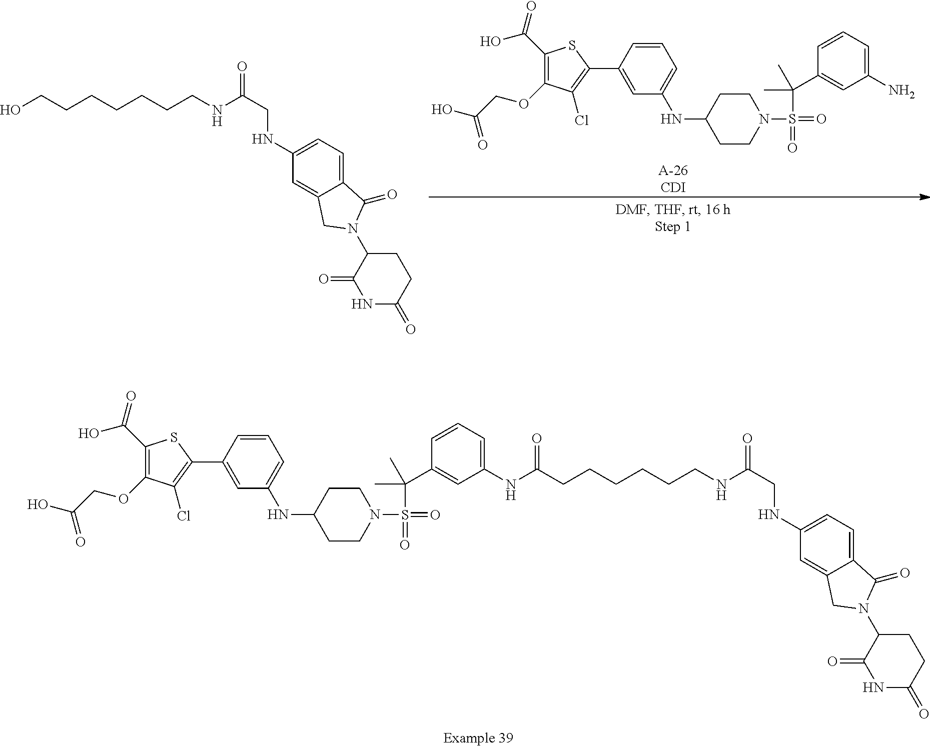 [SMILES: O=C(CNc1ccc2c(c1)CN(C1CCC(=O)NC1=O)C2=O)NCCCCCCCO ; CC(C)(c1cccc(N)c1)S(=O)(=O)N1CCC(Nc2cccc(-c3sc(C(=O)O)c(OCC(=O)O)c3Cl)c2)CC1 ; "A-26CDI" ; CC(C)(c1cccc(NC(=O)CCCCCCNC(=O)CNc2ccc3c(c2)CN(C2CCC(=O)NC2=O)C3=O)c1)S(=O)(=O)N1CCC(Nc2cccc(-c3sc(C(=O)O)c(OCC(=O)O)c3Cl)c2)CC1]

### Step 1: 3-(carboxymethoxy)-4-chloro-5-[3-[[1-[1-[3-[7-[[2-[[2-(2,6-dioxo-3-piperidyl)-1-oxo-isoindolin-5-yl]amino]acetyl]amino]heptanoylamino]phenyl]-1-methyl-ethyl]sulfonyl-4-piperidyl]amino]phenyl]thiophene-2-carboxylic acid (39)

3-(carboxymethoxy)-4-chloro-5-[3-[[1-[1-[3-[7-[[2-[[2-(2,6-dioxo-3-piperidyl)-1-oxo-isoindolin-5-yl]amino]acetyl]amino]heptanoylamino]phenyl]-1-methyl-ethyl]sulfonyl-4-piperidyl]amino]phenyl]thiophene-2-carboxylic acid (39, 7 mg, 6.65 μmol, 10% yield) was synthesized from 7-[[2-[[2-(2,6-dioxo-3-piperidyl)-1-oxo-isoindolin-5-yl]amino]acetyl]amino]heptanoic acid (B-62) and 5-[3-[[1-[1-(3-aminophenyl)-1-methyl-ethyl]sulfonyl-4-piperidyl]amino]phenyl]-3-(carboxymethoxy)-4-chloro-thiophene-2-carboxylic acid (A-26) in a similar fashion to Example 23, except using 1:1 THF:DMF as the solvent. The residue was purified by reverse phase preparative HPLC [Purification method: Column: SunFire C18 (19×150 mm) 5 μm, Mobile phase A: 0.1% TFA in water; Mobile phase B: MeCN]. UPLC-MS (ES+): m/z 1034.9 [M+H]⁺. ¹H NMR (400 MHz, DMSO-d₆) δ 10.92 (s, 1H), 9.92 (s, 1H), 7.91 (t, J=5.7 Hz, 1H), 7.79 (s, 1H), 7.68 (d, J=7.8 Hz, 1H), 7.41 (d, J=8.3 Hz, 1H), 7.34-7.24 (m, 2H), 7.19 (t, J=7.9 Hz, 1H), 6.86-6.83 (m, 1H), 6.80 (d, J=7.6 Hz, 1H), 6.66 (dd, J=8.4, 2.1 Hz, 2H), 6.58 (d, J=2.0 Hz, 1H), 5.01 (dd, J=13.3, 5.0 Hz, 1H), 4.91 (s, 2H), 4.27 (d, J=16.7 Hz, 1H), 4.14 (d, J=16.8 Hz, 1H), 3.70 (s, 2H), 3.41-3.24 (m, 4H), 3.07 (q, J=6.5 Hz, 2H), 2.94-2.79 (m, 3H), 2.57 (s, 1H), 2.40-2.35 (m, 1H), 2.29 (t, J=7.5 Hz, 2H), 1.99-1.88 (m, 1H), 1.86-1.76 (m, 2H), 1.69 (s, 6H), 1.55 (t, J=7.5 Hz, 2H), 1.45-1.34 (m, 2H), 1.30-1.19 (m, 6H).

### Example 40

### 3-(carboxymethoxy)-4-chloro-5-[3-[[1-[1-[3-[7-[[2-[[2-(2,6-dioxo-3-piperidyl)-1-oxo-isoindolin-4-yl]amino]acetyl]amino]heptanoylamino]phenyl]-1-methyl-ethyl]sulfonyl-4-piperidyl]amino]phenyl]thiophene-2-carboxylic acid (40)

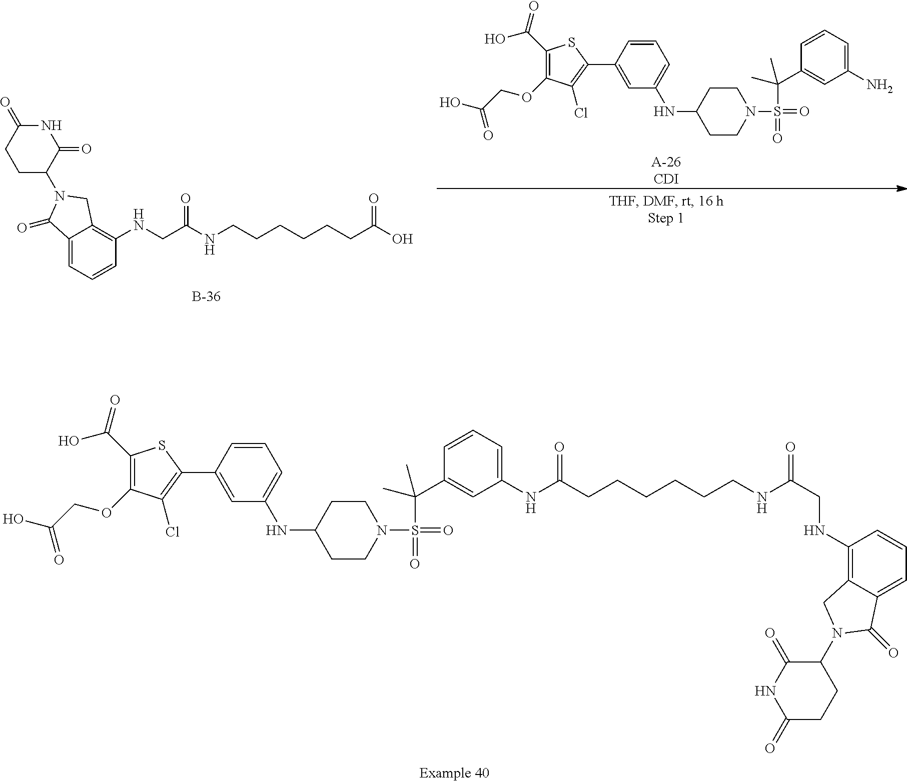

### Step 1: 3-(carboxymethoxy)-4-chloro-5-[3-[[1-[1-[3-[7-[[2-[[2-(2,6-dioxo-3-piperidyl)-1-oxo-isoindolin-4-yl]amino]acetyl]amino]heptanoylamino]phenyl]-1-methyl-ethyl]sulfonyl-4-piperidyl]amino]phenyl]thiophene-2-carboxylic acid (40)

3-(carboxymethoxy)-4-chloro-5-[3-[[1-[1-[3-[7-[[2-[[2-(2,6-dioxo-3-piperidyl)-1-oxo-isoindolin-4-yl]amino]acetyl]amino]heptanoylamino]phenyl]-1-methyl-ethyl]sulfonyl-4-piperidyl]amino]phenyl]thiophene-2-carboxylic acid (40, 7 mg, 6.63 μmol, 10% yield, TFA Salt) was synthesized from 7-[[2-[[2-(2,6-dioxo-3-piperidyl)-1-oxo-isoindolin-4-yl]amino]acetyl]amino]heptanoic acid (B-36) and 5-[3-[[1-[1-(3-aminophenyl)-1-methyl-ethyl]sulfonyl-4-piperidyl]amino]phenyl]-3-(carboxymethoxy)-4-chloro-thiophene-2-carboxylic acid (A-26) in a similar fashion to Example 23, except using 1:1 THF:DMF as the solvent. The residue was purified by reverse phase preparative HPLC [Purification method: Column: SunFire C18 (19×150 mm) 5 μm, Mobile phase A: 0.1% TFA in water; Mobile phase B: MeCN]. LCMS (ES+): m/z 1034.2 [M+H]⁺. ¹H NMR (400 MHz, DMSO-d₆) δ 11.01 (s, 1H), 9.93 (s, 1H), 7.93 (t, J=5.8 Hz, 1H), 7.79 (s, 1H), 7.68 (d, J=7.8 Hz, 1H), 7.34-7.24 (m, 3H), 7.18 (t, J=7.9 Hz, 1H), 6.97 (d, J=7.4 Hz, 1H), 6.87-6.83 (m, 1H), 6.80 (d, J=7.7 Hz, 1H), 6.69-6.63 (m, 1H), 6.55 (d, J=8.0 Hz, 1H), 5.12 (dd, J=13.3, 5.1 Hz, 1H), 4.91 (s, 2H), 4.28 (d, J=17.1 Hz, 1H), 4.18 (d, J=17.1 Hz, 1H), 3.72 (s, 2H), 3.41-3.23 (m, 3H), 3.05 (q, J=6.6 Hz, 2H), 2.99-2.75 (m, 3H), 2.65-2.57 (m, 2H), 2.31-2.24 (m, 2H), 2.08-1.97 (m, 1H), 1.85-1.76 (m, 2H), 1.69 (s, 6H), 1.61-1.49 (m, 2H), 1.43-1.32 (m, 2H), 1.31-1.15 (m, 6H).

### Example 41

### 3-(carboxymethoxy)-4-chloro-5-(3-(((4S)-1-((3-(1-((2-(2,6-dioxopiperidin-3-yl)-1-oxoisoindolin-5-yl)glycyl)piperidine-4-carboxamido)benzyl)sulfonyl)-2,2-dimethylpiperidin-4-yl)amino)phenyl)thiophene-2-carboxylic acid (41)

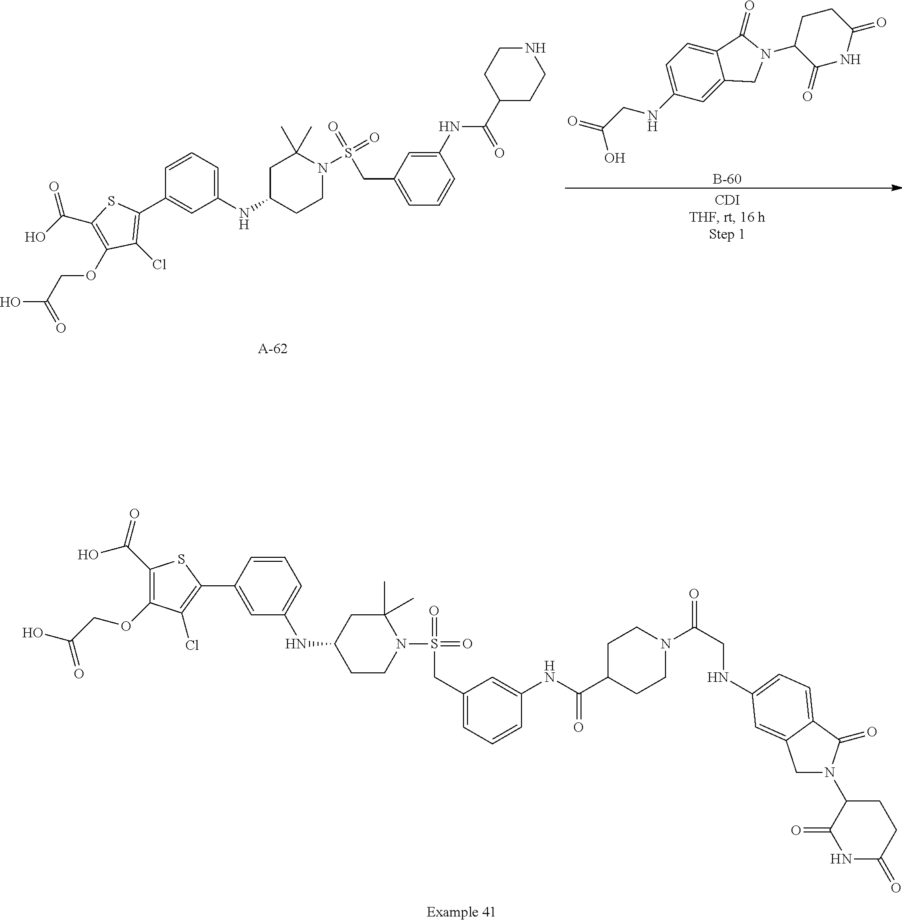

### Step 1: 3-(carboxymethoxy)-4-chloro-5-(3-(((4S)-1-((3-(1-((2-(2,6-dioxopiperidin-3-yl)-1-oxoisoindolin-5-yl)glycyl)piperidine-4-carboxamido)benzyl)sulfonyl)-2,2-dimethylpiperidin-4-yl)amino)phenyl)thiophene-2-carboxylic acid (41)

3-(carboxymethoxy)-4-chloro-5-(3-(((4S)-1-((3-(1-((2-(2,6-dioxopiperidin-3-yl)-1-oxoisoindolin-5-yl)glycyl)piperidine-4-carboxamido)benzyl)sulfonyl)-2,2-dimethylpiperidin-4-yl)amino)phenyl)thiophene-2-carboxylic acid (41, 0.014 g, 13.03 μmol, 14% yield, TFA salt) was synthesized from 2-[[2-(2,6-dioxo-3-piperidyl)-1-oxo-isoindolin-5-yl]amino]acetic acid (B-60) and 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62) in a similar fashion to Example 23. The residue was purified by reverse phase preparative HPLC [Purification method: Column: SunFire C18 (19×150 mm) 5 μm, Mobile phase A: 0.1% TFA in water; Mobile phase B: MeCN]. LCMS (ES+): m/z 1018.0 [M+H]⁺. ¹H NMR (400 MHz, DMSO-d₆) δ 10.93 (s, 1H), 10.02 (s, 1H), 7.69 (s, 1H), 7.60 (d, J=8.2 Hz, 1H), 7.40 (d, J=8.4 Hz, 1H), 7.30 (t, J=7.8 Hz, 1H), 7.19 (t, J=7.9 Hz, 1H), 7.07 (d, J=7.5 Hz, 1H), 6.86 (s, 1H), 6.80 (d, J=8.0 Hz, 2H), 6.75 (s, 1H), 6.68 (d, J=8.2 Hz, 1H), 5.69 (s, 1H), 5.02 (dd, J=13.1, 5.0 Hz, 1H), 4.91 (s, 2H), 4.48-4.36 (m, 2H), 4.33-4.23 (m, 2H), 4.15 (d, J=16.6 Hz, 1H), 4.08-3.95 (m, 3H), 3.18-3.04 (m, 3H), 2.96-2.83 (m, 1H), 2.75-2.58 (m, 3H), 2.42-2.35 (m, 1H), 1.99-1.91 (m, 1H), 1.91-1.75 (m, 5H), 1.74-1.61 (m, 1H), 1.56-1.44 (m, 4H), 1.39 (s, 4H), 1.23 (s, 1H), 1.18-1.02 (m, 1H).

### Example 42

### 3-(carboxymethoxy)-4-chloro-5-(3-((1-(((3-(2-((2-(2,6-dioxopiperidin-3-yl)-1,3-dioxoisoindolin-5-yl)amino)acetamido)phenyl)(phenyl)methyl)sulfonyl)piperidin-4-yl)amino)phenyl)thiophene-2-carboxylic acid (42)

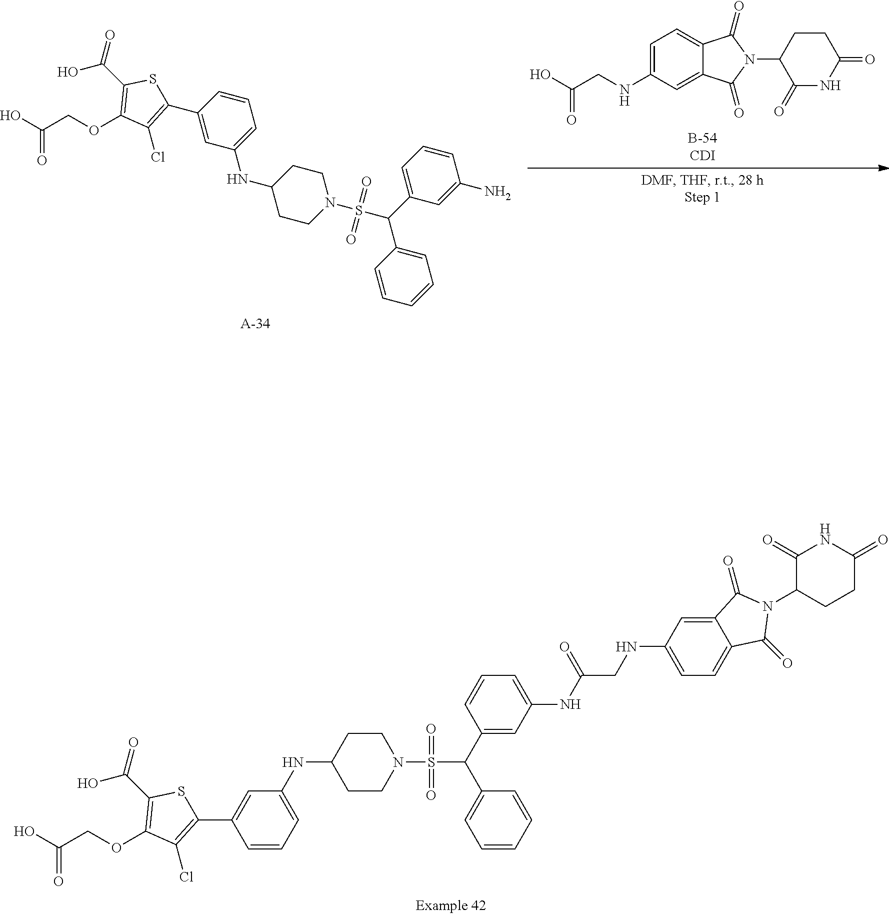 [SMILES: Nc1cccc(C(c2ccccc2)S(=O)(=O)N2CCC(Nc3cccc(-c4sc(C(=O)O)c(OCC(=O)O)c4Cl)c3)CC2)c1 ; O=C(O)CNc1ccc2c(c1)C(=O)N(C1CCC(=O)NC1=O)C2=O ; "A-34" ; O=C(O)COc1c(C(=O)O)sc(-c2cccc(NC3CCN(S(=O)(=O)C(c4ccccc4)c4cccc(NC(=O)CNc5ccc6c(c5)C(=O)N(C5CCC(=O)NC5=O)C6=O)c4)CC3)c2)c1Cl]

### Step 1: 3-(carboxymethoxy)-4-chloro-5-(3-((1-(((3-(2-((2-(2,6-dioxopiperidin-3-yl)-1,3-dioxoisoindolin-5-yl)amino)acetamido)phenyl)(phenyl)methyl)sulfonyl)piperidin-4-yl)amino)phenyl)thiophene-2-carboxylic acid (42)

3-(carboxymethoxy)-4-chloro-5-(3-((1-(((3-(2-((2-(2,6-dioxopiperidin-3-yl)-1,3-dioxoisoindolin-5-yl)amino)acetamido)phenyl)(phenyl)methyl)sulfonyl)piperidin-4-yl)amino)phenyl)thiophene-2-carboxylic acid (42, 22 mg, 0.022 mmol, 7% yield, TFA salt) was synthesized from 2-[[2-(2,6-dioxo-3-piperidyl)-1,3-dioxo-isoindolin-5-yl]amino]acetic acid (B-54) and 5-[3-[[1-[(3-aminophenyl)-phenyl-methyl]sulfonyl-4-piperidyl]amino]phenyl]-3-(carboxymethoxy)-4-chloro-thiophene-2-carboxylic acid (A-34) in a similar fashion to Example 23, except using 1:1 THF:DMF as the solvent. The residue was purified by reverse phase preparative HPLC [Purification method: Column: SunFire C18 (19×150 mm) 5 μm, Mobile phase A: 0.1% TFA in water; Mobile phase B: MeCN]. LCMS (ES+): m/z 969.2 [M+H]⁺. ¹H-NMR (400 MHz, DMSO-d₆): δ 11.07 (s, 1H), 10.27 (s, 1H), 7.99 (s, 1H), 7.70-7.59 (m, 4H), 7.43-7.34 (m, 5H), 7.20-7.16 (m, 1H), 7.09-7.02 (m, 1H), 6.99-6.89 (m, 1H), 6.75-6.88 (m, 2H), 6.21-6.12 (m, 1H), 5.86 (d, J=2.40 Hz, 1H), 5.06-5.03 (m, 1H), 4.95-4.92 (m, 2H), 4.10-4.10 (m, 1H), 3.80-3.70 (m, 4H), 3.29-3.28 (m, 2H), 2.95-2.80 (m, 1H), 2.10-1.94 (m, 1H), 1.85-1.75 (m, 2H), 1.24-1.18 (m, 2H).

### Example 43

### 3-(carboxymethoxy)-4-chloro-5-[3-[[(4S)-1-[[3-[[1-[2-[[2-(2,6-dioxo-3-piperidyl)-1,3-dioxo-isoindolin-5-yl]amino]acetyl]piperidine-4-carbonyl]amino]phenyl]methylsulfonyl]-2,2-dimethyl-4-piperidyl]amino]phenyl]thiophene-2-carboxylic acid (43)

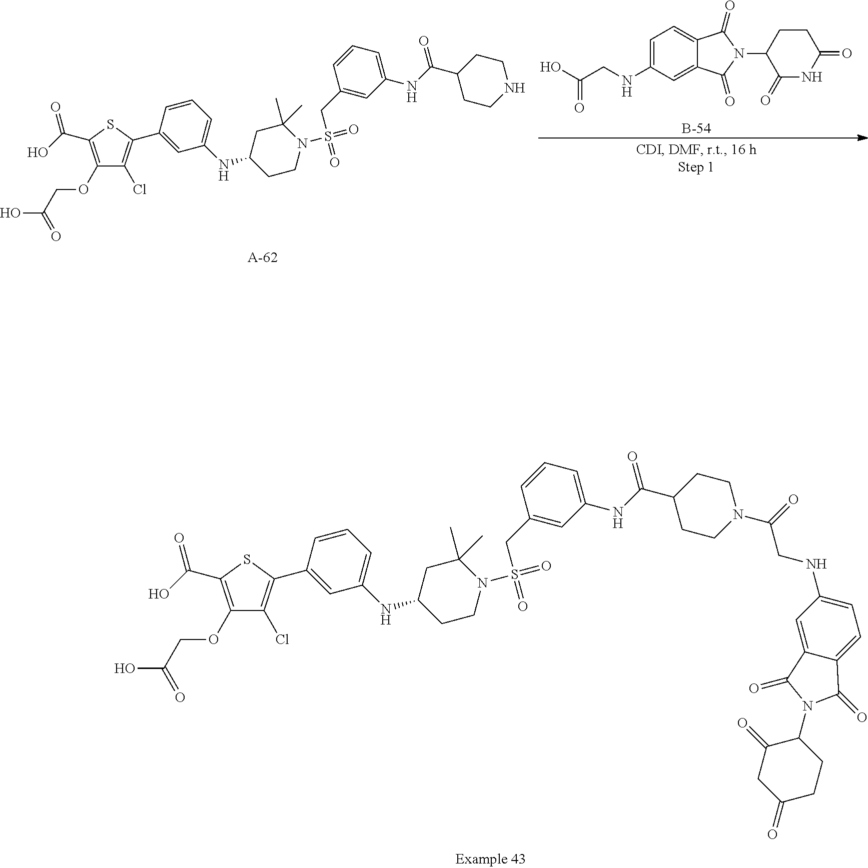

### Step 1: 3-(carboxymethoxy)-4-chloro-5-[3-[[(4S)-1-[[3-[[1-[2-[[2-(2,6-dioxo-3-piperidyl)-1,3-dioxo-isoindolin-5-yl]amino]acetyl]piperidine-4-carbonyl]amino]phenyl]methylsulfonyl]-2,2-dimethyl-4-piperidyl]amino]phenyl]thiophene-2-carboxylic acid (43)

3-(carboxymethoxy)-4-chloro-5-[3-[[(4S)-1-[3-[[1-[2-[[2-(2,6-dioxo-3-piperidyl)-1,3-dioxo-isoindolin-5-yl]amino]acetyl]piperidine-4-carbonyl]amino]phenyl]methylsulfonyl]-2,2-dimethyl-4-piperidyl]amino]phenyl]thiophene-2-carboxylic acid (43, 9.59 mg, 8.93 μmol, 18% yield, TFA salt) was synthesized from 2-[[2-(2,6-dioxo-3-piperidyl)-1,3-dioxo-isoindolin-5-yl]amino]acetic acid (B-54) and 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62) in a similar fashion to Example 23, except using 1:1 THF:DMF as the solvent. The residue was purified by reverse phase preparative HPLC [Purification method: Column: SunFire C18 (19×150 mm) 5 μm, Mobile phase A: 0.1% TFA in water; Mobile phase B: MeCN]. LCMS (ES+): m/z 1032.3 [M+H]⁺. ¹H NMR (400 MHz, DMSO-d₆) δ 11.07 (s, 1H), 10.04 (s, 1H), 7.69 (s, 1H), 7.63-7.55 (m, 2H), 7.30 (t, J=7.9 Hz, 1H), 7.19 (t, J=7.9 Hz, 1H), 7.12 (s, 1H), 7.11-7.05 (m, 2H), 7.05-6.98 (m, 1H), 6.85 (s, 1H), 6.79 (d, J=7.6 Hz, 1H), 6.68 (d, J=8.3 Hz, 1H), 5.69 (s, 1H), 5.04 (dd, J=12.9, 5.4 Hz, 1H), 4.90 (s, 2H), 4.47-4.35 (m, 2H), 4.28 (d, J=13.7 Hz, 1H), 4.23-4.07 (m, 3H), 4.05-3.96 (m, 1H), 3.18-3.05 (m, 2H), 2.94-2.80 (m, 2H), 2.76-2.57 (m, 3H), 2.04-1.95 (m, 2H), 1.91-1.75 (m, 4H), 1.73-1.60 (m, 1H), 1.55-1.42 (m, 3H), 1.41-1.35 (m, 4H), 1.18-1.04 (m, 1H).

### Example 44

### 3-(Carboxymethoxy)-4-chloro-5-(3-(((4S)-1-((3-(1-((2-(2,6-dioxopiperidin-3-yl)-1-oxoisoindolin-4-yl)glycyl) azetidine-3-carboxamido)benzyl)sulfonyl)-2,2-dimethylpiperidin-4-yl)amino)phenyl)thiophene-2-carboxylic acid (44)

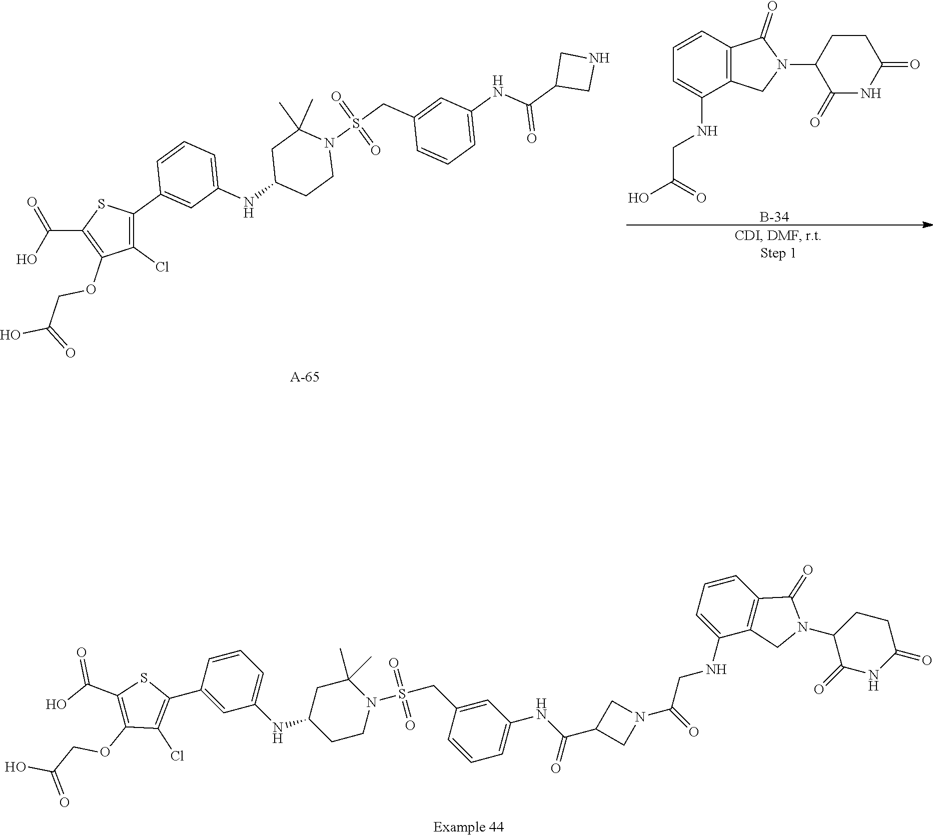

### Step 1: 3-(carboxymethoxy)-4-chloro-5-(3-(((4S)-1-((3-(1-((2-(2,6-dioxopiperidin-3-yl)-1-oxoisoindolin-4-yl)glycyl) azetidine-3-carboxamido)benzyl)sulfonyl)-2,2-dimethylpiperidin-4-yl)amino)phenyl)thiophene-2-carboxylic acid (44)

3-(carboxymethoxy)-4-chloro-5-(3-(((4S)-1-((3-(1-((2-(2,6-dioxopiperidin-3-yl)-1-oxoisoindolin-4-yl)glycyl) azetidine-3-carboxamido)benzyl)sulfonyl)-2,2-dimethylpiperidin-4-yl)amino)phenyl)thiophene-2-carboxylic acid (44, 19 mg, 16.36 μmol, 17% yield, TFA salt) was synthesized from 2-[[2-(2,6-dioxo-3-piperidyl)-1-oxo-isoindolin-4-yl]amino]acetic acid (B-34) and 5-[3-[[(4S)-1-[[3-(azetidine-3-carbonylamino)phenyl]methylsulfonyl]-2,2-dimethyl-4-piperidyl]amino]phenyl]-3-(carboxymethoxy)-4-chloro-thiophene-2-carboxylic acid (A-65) in a similar fashion to Example 23, except using 1:1 THF:DMF as the solvent. The residue was purified by reverse phase preparative HPLC [Purification method: Column; SunFire C18 (19×150 mm) 5 μm, Mobile phase A: 0.1% TFA in water; Mobile phase B: MeCN]. LCMS (ES+): m/z 990.3 [M+H]⁺. ¹H NMR (400 MHz, DMSO-d₆) δ 11.00 (s, 1H), 10.16 (s, 1H), 7.67 (s, 1H), 7.65 (d, J=8.7 Hz, 1H), 7.31 (ddd, J=15.5, 7.8 Hz, 2H), 7.22-7.16 (m, 1H), 7.11 (d, J=7.6 Hz, 1H), 6.98 (d, J=7.4 Hz, 1H), 6.89-6.86 (m, 1H), 6.80 (d, J=7.6 Hz, 1H), 6.71-6.65 (m, 2H), 5.11 (dd, J=13.2, 5.1 Hz, 1H), 4.91 (s, 2H), 4.44-4.29 (m, 4H), 4.27 (d, J=9.1 Hz, 1H), 4.16 (d, J=17.1 Hz, 1H), 4.09-3.98 (m, 2H), 3.85 (s, 2H), 3.64-3.58 (m, 2H), 3.18-3.06 (m, 2H), 2.98-2.85 (m, 2H), 2.08-1.99 (m, 1H), 1.88 (d, J=11.7 Hz, 1H), 1.79 (d, J=12.7 Hz, 1H), 1.47 (s, 3H), 1.39 (s, 3H), 1.18-1.01 (m, 2H).

### Example 44a and Example 44b

### 3-(carboxymethoxy)-4-chloro-5-[3-[[(4S)-2,2-dimethyl-1-[[3-[[1-[2-[[1-oxo-2-[(3R)-2,6-dioxo-3-piperidyl]isoindolin-4-yl]amino]acetyl]azetidine-3-carbonyl]amino]phenyl]methylsulfonyl]-4-piperidyl]amino]phenyl]thiophene-2-carboxylic acid (44a) and 3-(carboxymethoxy)-4-chloro-5-[3-[[(4S)-2,2-dimethyl-1-[[3-[[1-[2-[[1-oxo-2-[(3S)-2,6-dioxo-3-piperidyl]isoindolin-4-yl]amino]acetyl]azetidine-3-carbonyl]amino]phenyl]methylsulfonyl]-4-piperidyl]amino]phenyl]thiophene-2-carboxylic acid (44b)

Configurations are arbitrarily assigned.

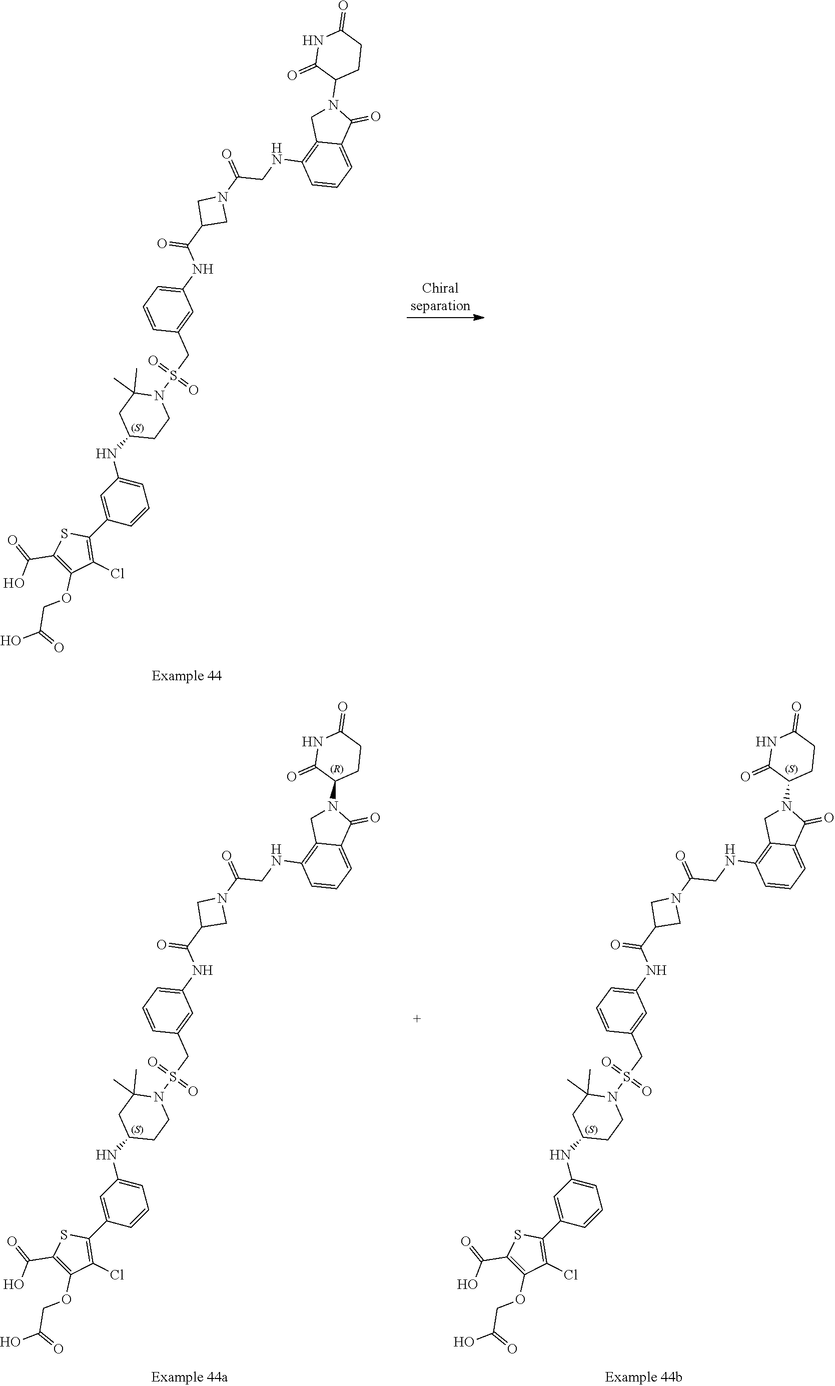

3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]azetidine-3-carbonyl]amino]phenyl]methylsulfonyl]-2,2-dimethyl-4-piperidyl]amino]phenyl]thiophene-2-carboxylic acid (44, 400 mg, 0.36 mmol, TFA salt) was subjected to chiral SFC purification to separate the diastereomers. Method details: Column Name: Chiralpak AS-H, Flowrate: 5 mL/min, Co-Solvent: 50%, Co-Solvent Name: 0.2% TFA in IPA:ACN (1:1), Injected Volume: 15 μL, Temperature: 35° C., Outlet Pressure: 100 bar.

### Chiral SFC:

### Method of Analysis

Instrument: PIC 175
Column: Chiralpak AS-H (250×30) mm, 5 μm;
Mobile Phase: CO₂: 0.2% Trifluoracetic acid in Isopropyl alcohol:Acetonitrile (1:1):(50:50)
Total Flow: 90 g/min
Back pressure: 100 bar
Wave length: 220 nm
Cycle time: 14 min

400 mg of Example 44 was dissolved in 8 mL of ACN and injected 500 L/injection.

After SFC purification, the first eluted fraction at RT: 2.3 min was concentrated under reduced pressure at 40° C. and purified by reverse-phase column chromatography [Silicycle C18 column; Mobile Phase A: 0.1% TFA in water and MeCN] to afford 3-(carboxymethoxy)-4-chloro-5-[3-[[(4S)-2,2-dimethyl-1-[[3-[1-[2-[1-oxo-2-[(3R)-2,6-dioxo-3-piperidyl]isoindolin-4-yl]amino]acetyl]azetidine-3-carbonyl]amino]phenyl]methylsulfonyl]-4-piperidyl]amino]phenyl]thiophene-2-carboxylic acid (44a, 120 mg, 106.97 μmol, 14% yield, TFA salt) as an off-white solid.

### Fraction 1 (44a):

¹H-NMR (400 MHz, DMSO-d6): δ 11.00 (s, 1H), 10.17 (s, 1H), 7.68 (s, 1H), 7.65 (d, J=8.00 Hz, 1H), 7.36-7.28 (m, 2H), 7.20 (t, J=8.00 Hz, 1H), 7.11 (d, J=7.60 Hz, 1H), 6.99 (d, J=7.20 Hz, 1H), 6.88 (s, 1H), 6.81 (d, J=7.60 Hz, 1H), 6.69 (t, J=7.20 Hz, 2H), 5.11 (dd, J=5.20, 13.20 Hz, 1H), 4.92 (s, 2H), 4.43-4.15 (m, 6H), 4.07-4.03 (m, 2H), 3.85 (s, 2H), 3.20-3.10 (m, 1H), 3.00-2.87 (m, 1H), 2.62-2.58 (m, 1H), 2.41-2.28 (m, 1H), 2.09-2.00 (m, 1H), 1.92-1.72 (m, 2H), 1.49-1.35 (m, 7H), 1.20-1.08 (m, 1H). Note:

3 aliphatic protons are obscured by the solvent signal.

LCMS (ES+): m/z 990.1 [M+H]⁺

Similarly, the second eluted fraction at RT: 3.4 min was concentrated under reduced pressure at 40° C. and purified by reverse-phase column chromatography [Silicycle C18 column; Mobile Phase A: 0.1% TFA in water and MeCN] to afford 3-(carboxymethoxy)-4-chloro-5-[3-[[(4S)-2,2-dimethyl-1-[[3-[1-[2-[[1-oxo-2-[(3S)-2,6-dioxo-3-piperidyl]isoindolin-4-yl]amino]acetyl]azetidine-3-carbonyl]amino]phenyl]methylsulfonyl]-4-piperidyl]amino]phenyl]thiophene-2-carboxylic acid (44b, 52 mg, 46.47 μmol, 6% yield, TFA salt) as an off-white solid.

### Fraction 2 (44b):

¹H-NMR (400 MHz, DMSO-d6): δ 11.00 (s, 1H), 10.17 (s, 1H), 7.68 (s, 1H), 7.65 (d, J=8.40 Hz, 1H), 7.36-7.28 (m, 2H), 7.20 (t, J=7.60 Hz, 1H), 7.11 (d, J=8.00 Hz, 1H), 6.99 (d, J=7.20 Hz, 1H), 6.88 (s, 1H), 6.81 (d, J=7.60 Hz, 1H), 6.69 (t, J=8.00 Hz, 2H), 5.11 (dd, J=5.20, 13.20 Hz, 1H), 4.92 (s, 2H), 4.43-4.15 (m, 6H), 4.07-4.03 (m, 2H), 3.86 (s, 2H), 3.20-3.10 (m, 1H), 3.00-2.88 (m, 1H), 2.65-2.59 (m, 1H), 2.41-2.29 (m, 1H), 2.09-2.00 (m, 1H), 1.92-1.75 (m, 2H), 1.50-1.35 (m, 7H), 1.20-1.09 (m, 1H). Note:

3 aliphatic protons are obscured by the solvent signal.

LCMS (ES+): m/z 990.1 [M+H]⁺.

### Example 45

3-(carboxymethoxy)-4-chloro-5-[3-[[1-[[3-[[2-[4-[[2-(2,6-dioxo-3-piperidyl)-1-oxo-isoindolin-5-yl]-1-piperidyl]acetyl]amino]phenyl]methylsulfonyl]-4-piperidyl]amino]phenyl]thiophene-2-carboxylic acid (45)

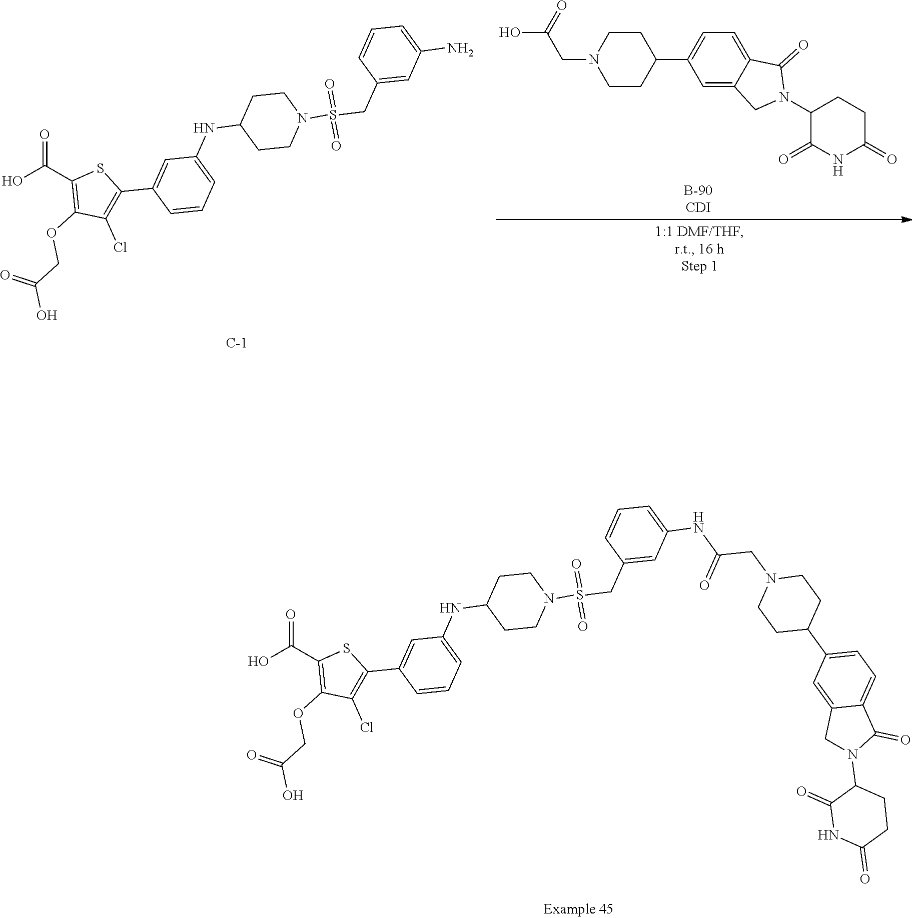

### Step 1: 3-(carboxymethoxy)-4-chloro-5-[3-[[1-[[3-[[2-[4-[[2-(2,6-dioxo-3-piperidyl)-1-oxo-isoindolin-5-yl]-1-piperidyl]acetyl]amino]phenyl]methylsulfonyl]-4-piperidyl]amino]phenyl]thiophene-2-carboxylic acid (45)

3-(carboxymethoxy)-4-chloro-5-[3-[[1-[[3-[2-[4-[[2-(2,6-dioxo-3-piperidyl)-1-oxo-isoindolin-5-yl]-1-piperidyl]acetyl]amino]phenyl]methylsulfonyl]-4-piperidyl]amino]phenyl]thiophene-2-carboxylic acid (45, 23 mg, 24.18 μmol, 19% yield, TFA salt) was synthesized from 2-[4-[[2-(2,6-dioxo-3-piperidyl)-1-oxo-isoindolin-5-yl]-1-piperidyl]acetic acid (B-90) and 5-[3-[[1-[(3-aminophenyl)methylsulfonyl]-4-piperidyl]amino]phenyl]-3-(carboxymethoxy)-4-chloro-thiophene-2-carboxylic acid (C-1) in a similar fashion to Example 23, except using 1:1 THF:DMF as the solvent. The residue was purified by reverse phase preparative HPLC [Purification method: Column: SunFire C18 (19×150 mm) 5 μm, Mobile phase A: 0.1% TFA in water; Mobile phase B: MeCN]. LCMS (ES+): m/z 946.5 [M+H]⁺. ¹H NMR (400 MHz, DMSO-d₆) δ 10.99 (s, 1H), 10.68 (s, 1H), 7.72 (d, J=7.8 Hz, 1H), 7.68-7.62 (m, 2H), 7.48 (s, 1H), 7.44-7.37 (m, 2H), 7.25-7.17 (m, 2H), 6.90-6.86 (m, 1H), 6.82 (d, J=7.6 Hz, 1H), 6.74-6.68 (m, 1H), 5.12 (dd, J=13.3, 5.1 Hz, 1H), 4.92 (s, 2H), 4.50-4.39 (m, 3H), 4.33 (d, J=17.4 Hz, 1H), 4.20 (s, 2H), 3.32-3.22 (m, 3H), 3.04-2.87 (m, 4H), 2.65-2.54 (m, 2H), 2.43-2.36 (m, 1H), 2.15-1.93 (m, 7H), 1.46-1.30 (m, 2H).

### Example 46

### 3-(carboxymethoxy)-4-chloro-5-(3-(((4S)-1-((3-(3-(4-((2-(2,6-dioxopiperidin-3-yl)-1-oxoisoindolin-4-yl)amino)piperidin-1-yl)propanamido)benzyl)sulfonyl)-2,2-dimethylpiperidin-4-yl)amino)phenyl)thiophene-2-carboxylic acid (46)

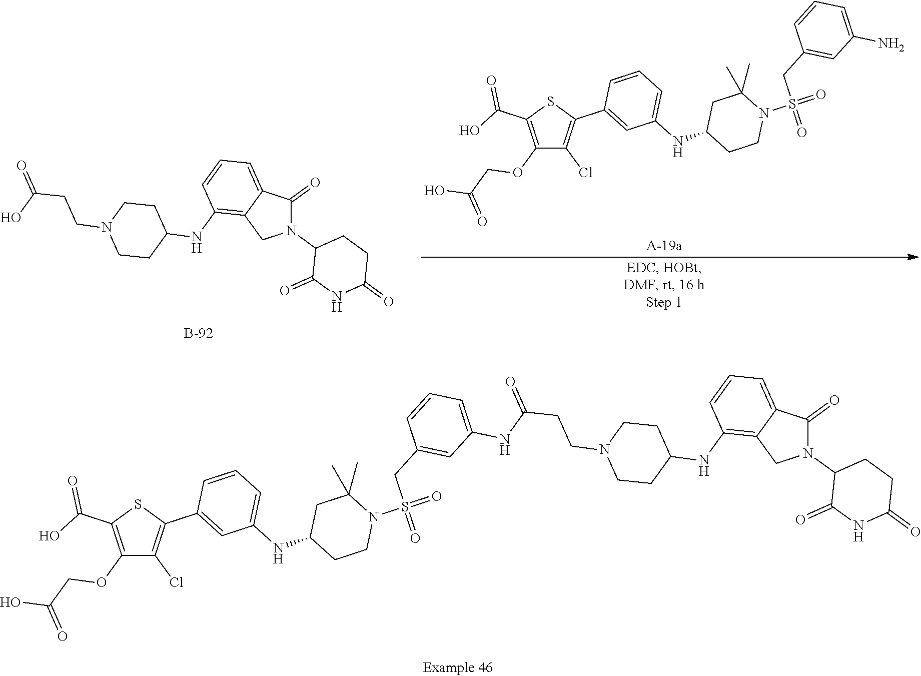

### Step 1: 3-(carboxymethoxy)-4-chloro-5-(3-(((4S)-1-((3-(3-(4-((2-(2,6-dioxopiperidin-3-yl)-1-oxoisoindolin-4-yl)amino)piperidin-1-yl)propanamido)benzyl)sulfonyl)-2,2-dimethylpiperidin-4-yl)amino)phenyl)thiophene-2-carboxylic acid (46)

Into a 25 mL single neck round bottom flask containing a well-stirred solution of 3-(4-((2-(2,6-dioxopiperidin-3-yl)-1-oxoisoindolin-4-yl)amino)piperidin-1-yl)propanoic acid (B-92, 80 mg, 193.03 μmol) in DMF (2 mL) was added EDC (111 mg, 579.08 μmol) followed by HOBt (26 mg, 193.03 μmol). The reaction mixture was stirred at room temperature for 3 h. Then 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 117 mg, 193.03 μmol) was added. The reaction mixture was stirred at room temperature for 16 h. The solvent was evaporated from the reaction mixture under reduced pressure. The obtained residue was subjected to reverse phase prep HPLC (Column: SunFire, 19×100 mm-5 μm; Mobile phase: 0.1% TFA in water/MeCN:water (90:10)) to obtain 3-(carboxymethoxy)-4-chloro-5-(3-(((4S)-1-((3-(3-(4-((2-(2,6-dioxopiperidin-3-yl)-1-oxoisoindolin-4-yl)amino)piperidin-1-yl)propanamido)benzyl)sulfonyl)-2,2-dimethylpiperidin-4-yl)amino)phenyl)thiophene-2-carboxylic acid (46, 5.51 mg, 5.13 μmol, 3% yield). LCMS (ES+): m/z 1004.9 [M+H]⁺. ¹H NMR (400 MHz, DMSO-d6): δ 11.04 (s, 1H), 10.27 (s, 1H), 9.25 (s, 1H), 7.62-7.64 (m, 2H), 7.28-7.30 (m, 2H), 7.20 (t, J=8.00 Hz, 1H), 7.11 (d, J=7.60 Hz, 1H), 6.96-6.98 (m, 1H), 6.86-6.88 (m, 1H), 6.80-6.81 (m, 3H), 6.69 (d, J=8.00 Hz, 1H), 5.50 (s, 1H), 5.11-5.12 (m, 1H), 4.91 (s, 2H), 4.11-4.15 (m, 4H), 3.35-3.43 (m, 5H), 3.13-3.16 (m, 2H), 2.85-2.87 (m, 3H), 2.64-2.67 (m, 1H), (t, J=Hz, 3H), 2.26-2.30 (m, 1H), 2.13-2.16 (m, 1H), 2.02-2.04 (m, 2H), 1.87-1.90 (m, 1H), 1.78-1.81 (m, 1H), 1.66-1.78 (m, 2H), 1.46 (s, 3H), 1.39 (s, 3H), 1.12-1.20 (m, 1H).

### Example 47

### 3-(carboxymethoxy)-4-chloro-5-[3-[[1-[[3-[3-[[2-(2,6-dioxo-3-piperidyl)-1-oxo-isoindolin-5-yl]propanoylamino]phenyl]methylsulfonyl]-4-piperidyl]amino]phenyl]thiophene-2-carboxylic acid (47)

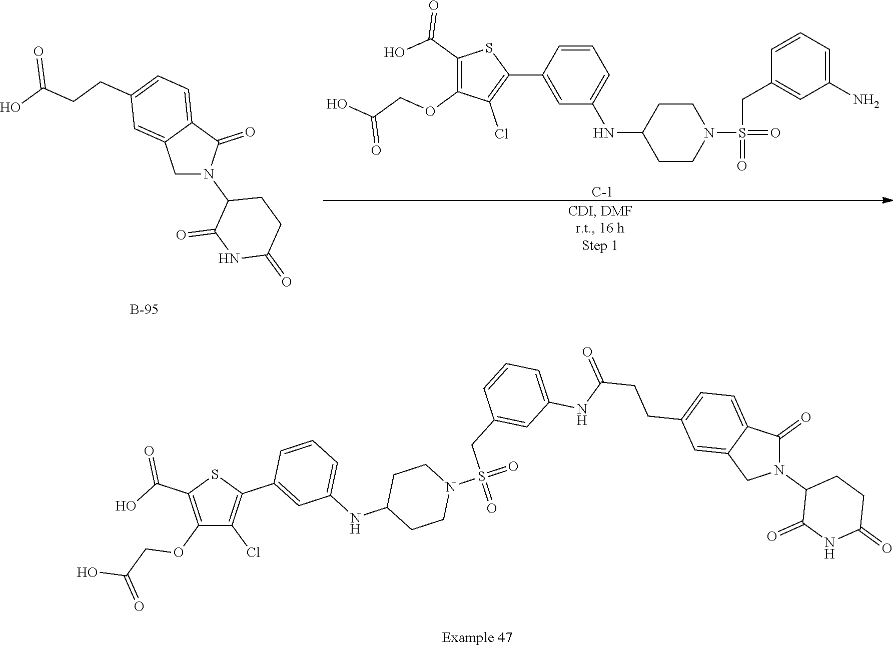

### Step 1: 3-(carboxymethoxy)-4-chloro-5-[3-[[1-[[3-[3-[[2-(2,6-dioxo-3-piperidyl)-1-oxo-isoindolin-5-yl]propanoylamino]phenyl]methylsulfonyl]-4-piperidyl]amino]phenyl]thiophene-2-carboxylic acid (47)

3-(carboxymethoxy)-4-chloro-5-[3-[[1-[[3-[3-[[2-(2,6-dioxo-3-piperidyl)-1-oxo-isoindolin-5-yl]propanoylamino]phenyl]methylsulfonyl]-4-piperidyl]amino]phenyl]thiophene-2-carboxylic acid (47, 30 mg, 33.69 μmol, 15% yield, TFA salt) was synthesized from 3-[[2-(2,6-dioxo-3-piperidyl)-1-oxo-isoindolin-5-yl]propanoic acid (B-95) and 5-[3-[[1-[(3-aminophenyl)methylsulfonyl]-4-piperidyl]amino]phenyl]-3-(carboxymethoxy)-4-chloro-thiophene-2-carboxylic acid (C-1) in a similar fashion to Example 23, except using 1:1 THF:DMF as the solvent. The residue was purified by reverse phase preparative HPLC [Purification method: Column: SunFire C18 (19×150 mm) 5 μm, Mobile phase A: 0.1% TFA in Water; Mobile phase B: Acetonitrile]. LCMS (ES+): m/z 877.6 [M+H]⁺. ¹H NMR (400 MHz, DMSO-d₆) δ 10.98 (s, 1H), 10.03 (s, 1H), 7.68-7.62 (m, 2H), 7.55 (d, J=8.2 Hz, 1H), 7.47 (s, 1H), 7.40 (d, J=7.9 Hz, 1H), 7.30 (t, J=7.9 Hz, 1H), 7.24-7.17 (m, 1H), 7.08 (d, J=7.5 Hz, 1H), 6.91-6.87 (m, 1H), 6.82 (d, J=7.5 Hz, 1H), 6.71 (d, J=8.2 Hz, 1H), 5.09 (dd, J=13.4, 5.1 Hz, 1H), 4.91 (s, 2H), 4.45-4.34 (m, 3H), 4.27 (d, J=17.2 Hz, 1H), 3.58-3.50 (m, 2H), 3.46-3.36 (m, 1H), 3.10-3.01 (m, 2H), 3.00-2.84 (m, 3H), 2.73-2.66 (m, 2H), 2.58-2.54 (m, 2H), 2.45-2.35 (m, 1H), 2.03-1.90 (m, 3H).

### Example 48

### 3-(carboxymethoxy)-4-chloro-5-(3-(((4S)-1-((3-(1-((2-(2,6-dioxopiperidin-3-yl)-1-oxoisoindolin-4-yl)glycyl)piperidine-4-carboxamido)benzyl)sulfonyl)-2,2-dimethylpiperidin-4-yl)amino)phenyl)thiophene-2-carboxylic acid (48)

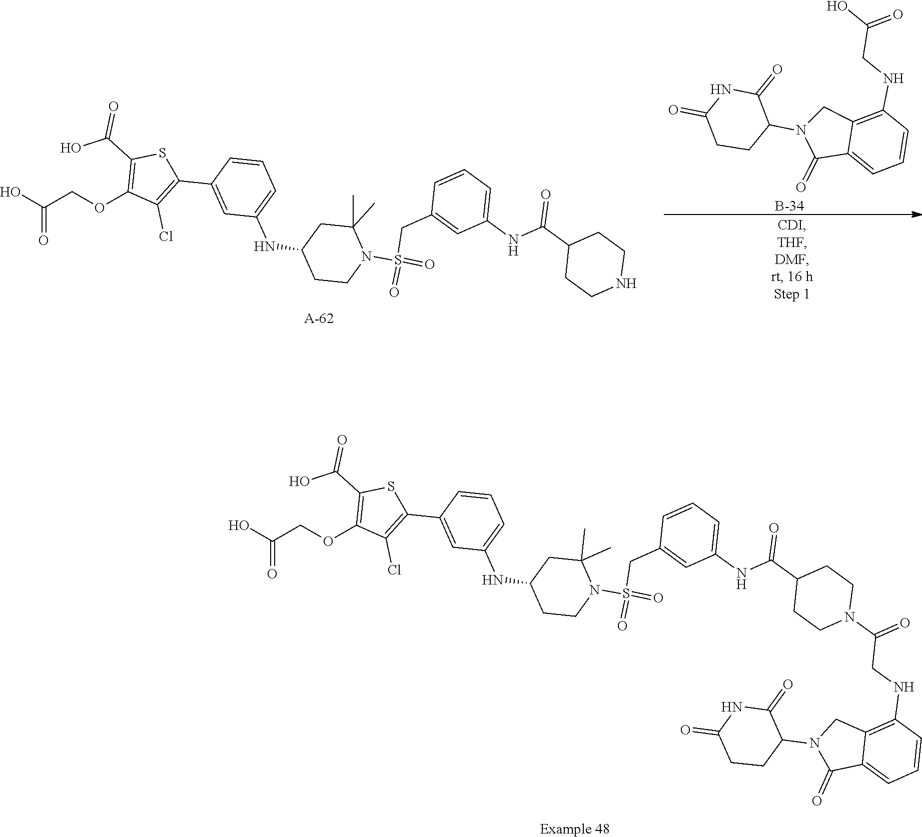

### Step 1: 3-(carboxymethoxy)-4-chloro-5-(3-(((4S)-1-((3-(1-((2-(2,6-dioxopiperidin-3-yl)-1-oxoisoindolin-4-yl)glycyl)piperidine-4-carboxamido)benzyl)sulfonyl)-2,2-dimethylpiperidin-4-yl)amino)phenyl)thiophene-2-carboxylic acid (48)

3-(carboxymethoxy)-4-chloro-5-(3-(((4S)-1-((3-(1-((2-(2,6-dioxopiperidin-3-yl)-1-oxoisoindolin-4-yl)glycyl)piperidine-4-carboxamido)benzyl)sulfonyl)-2,2-dimethylpiperidin-4-yl)amino)phenyl)thiophene-2-carboxylic acid (48, 8.2 mg, 7.01 μmol, 22% yield) was synthesized from 2-[[2-(2,6-dioxo-3-piperidyl)-1-oxo-isoindolin-4-yl]amino]acetic acid (B-34) and 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62) in a similar fashion to Example 23, except using 1:1 THF:DMF as the solvent. The residue was purified by reverse phase preparative HPLC (Column: SUNFIRE 18-OBD, water; Mobile phase A: 0.1% TFA in water, mobile phase B: MeCN). LCMS (ES+): m/z 1019.0 [M+H]⁺. ¹H NMR (400 MHz, DMSO-d₆) δ 11.00 (s, 1H), 10.03 (s, 1H), 7.69 (s, 1H), 7.60 (d, J=8.2 Hz, 1H), 7.34-7.25 (m, 2H), 7.19 (t, J=7.9 Hz, 1H), 7.08 (d, J=7.6 Hz, 1H), 6.98 (d, J=7.4 Hz, 1H), 6.86 (d, J=2.4 Hz, 1H), 6.79 (t, J=8.3 Hz, 2H), 6.71-6.65 (m, 1H), 5.70 (s, 1H), 5.57 (s, 1H), 5.12 (dd, J=13.3, 5.1 Hz, 1H), 4.91 (s, 2H), 4.46-4.36 (m, 2H), 4.34-4.26 (m, 2H), 4.18 (d, J=17.0 Hz, 1H), 4.10-3.98 (m, 3H), 3.17-3.04 (m, 3H), 2.98-2.86 (m, 1H), 2.71-2.57 (m, 4H), 2.42-2.35 (m, 1H), 2.07-1.97 (m, 1H), 1.91-1.72 (m, 5H), 1.66 (d, J=12.3 Hz, 1H), 1.47 (s, 3H), 1.39 (s, 3H), 1.30-1.20 (m, 1H), 1.15-1.02 (m, 1H).

### Example 49

### 3-(carboxymethoxy)-4-chloro-5-(3-((1-((3-(3-(1-(2,6-dioxopiperidin-3-yl)-3-methyl-2-oxo-2,3-dihydro-1H-benzo[d]imidazol-5-yl)propanamido)benzyl)sulfonyl)piperidin-4-yl)amino)phenyl)thiophene-2-carboxylic acid (49)

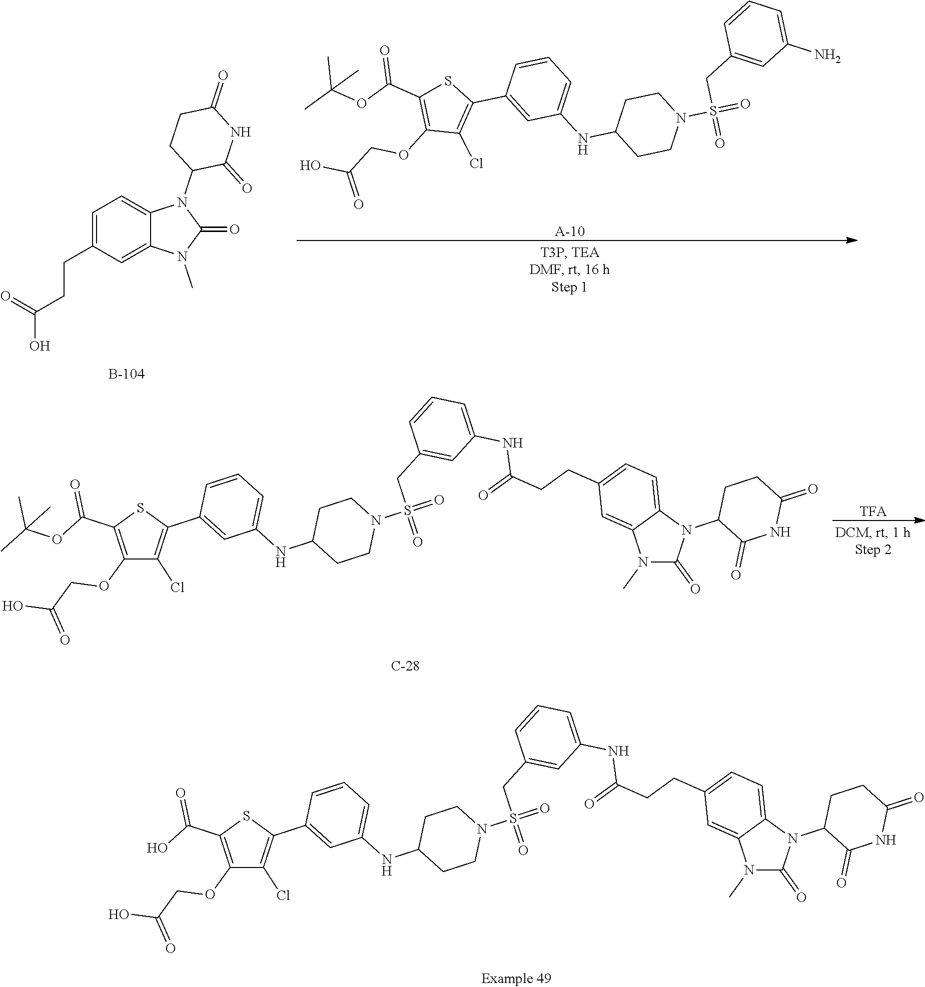

### Step 1: 2-((2-(tert-butoxycarbonyl)-4-chloro-5-(3-((1-((3-(3-(1-(2,6-dioxopiperidin-3-yl)-3-methyl-2-oxo-2,3-dihydro-1H-benzo[d]imidazol-5-yl)propanamido)benzyl) sulfonyl))piperidin-4-yl)amino)phenyl)thiophen-3-yl)oxy)acetic acid (C-28)

2-((2-(tert-butoxycarbonyl)-4-chloro-5-(3-((1-((3-(3-(1-(2,6-dioxopiperidin-3-yl)-3-methyl-2-oxo-2,3-dihydro-1H-benzo[d]imidazol-5-yl)propanamido)benzyl)sulfonyl)piperidin-4-yl)amino)phenyl)thiophen-3-yl)oxy)acetic acid (C-28, 100 mg, 27.38 μmol, 13% yield) was synthesized from 3-[1-(2,6-dioxo-3-piperidyl)-3-methyl-2-oxo-benzimidazol-5-yl]propanoic acid (B-104) and 2-[[5-[3-1 [1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Compound C-6. LCMS (ESI): m/z 949.8 [M+H]⁺.

### Step 2: 3-(carboxymethoxy)-4-chloro-5-(3-((1-((3-(3-(1-(2,6-dioxopiperidin-3-yl)-3-methyl-2-oxo-2,3-dihydro-1H-benzo[d]imidazol-5-yl)propanamido)benzyl)sulfonyl)piperidin-4-yl)amino)phenyl)thiophene-2-carboxylic acid (49)

3-(carboxymethoxy)-4-chloro-5-(3-((1-((3-(3-(1-(2,6-dioxopiperidin-3-yl)-3-methyl-2-oxo-2,3-dihydro-1H-benzo[d]imidazol-5-yl)propanamido)benzyl)sulfonyl)piperidin-4-yl)amino)phenyl)thiophene-2-carboxylic acid (49, 5.08 mg, 5.39 μmol, 19% yield, TFA salt) was synthesized from 2-((2-(tert-butoxy carbonyl)-4-chloro-5-(3-((1-((3-(3-(1-(2,6-dioxopiperidin-3-yl)-3-methyl-2-oxo-2,3-dihydro-1H-benzo[d]imidazol-5-yl)propanamido)benzyl)sulfonyl)piperidin-4-yl)amino)phenyl)thiophen-3-yl)oxy)acetic acid (C-28) in a similar fashion to Compound C-1. The residue was purified by reverse phase prep HPLC (Column: SunFire, 19×100 mm; 5 μm; Mobile phase: 0.1% TFA in water/MeCN:water (90:10)). MS (ESI): m/z 893.6 [M+H]⁺. ¹H NMR (400 MHz, DMSO-d₆) δ 11.08 (s, 1H), 10.02 (s, 1H), 7.69-7.64 (m, 1H), 7.57 (d, J=8.1 Hz, 1H), 7.35-7.27 (m, 1H), 7.25-7.17 (m, 1H), 7.12-7.05 (m, 2H), 7.04-6.98 (m, 1H), 6.95-6.86 (m, 2H), 6.82 (d, J=7.5 Hz, 1H), 6.71 (d, J=8.3 Hz, 1H), 5.37-5.28 (m, 1H), 4.91 (d, J=2.2 Hz, 2H), 4.36 (s, 2H), 3.30 (d, J=2.1 Hz, 3H), 3.02-2.83 (m, 6H), 2.74-2.60 (m, 4H), 2.05-1.88 (m, 4H), 1.44-1.29 (m, 2H).

### Example 50

### 3-(carboxymethoxy)-4-chloro-5-[3-[[1-[[3-[3-[4-[[2-(2,6-dioxo-3-piperidyl)-1-oxo-isoindolin-4-yl]amino]-1-piperidyl]propanoylamino]phenyl]methylsulfonyl]-4-piperidyl]amino]phenyl]thiophene-2-carboxylic acid (50)

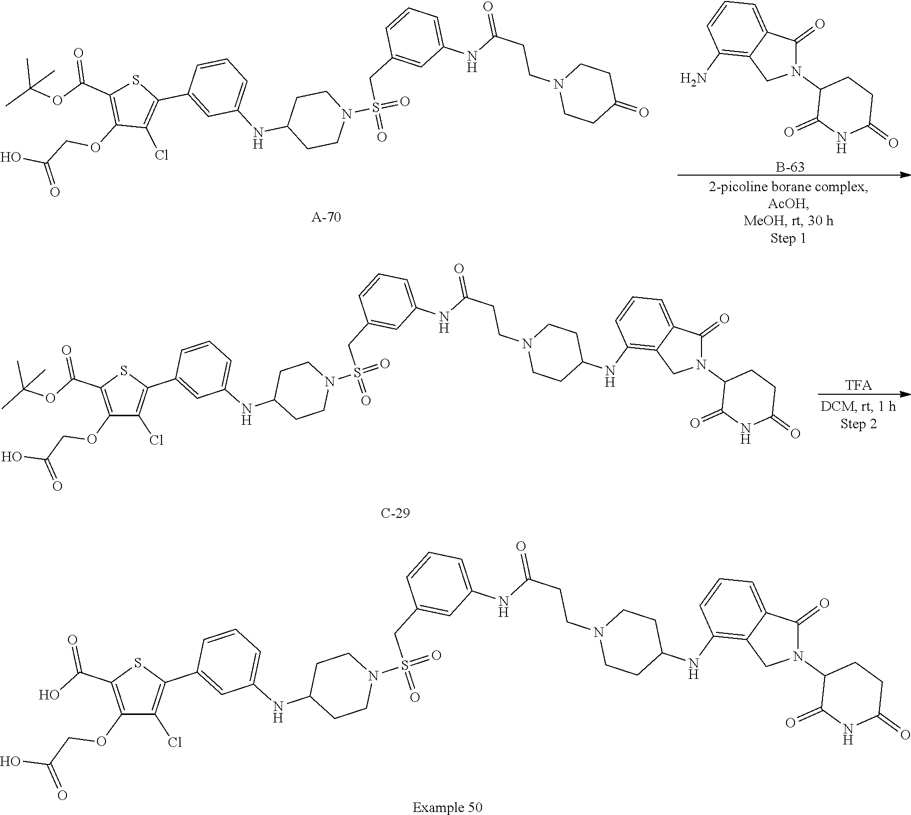

### Step 1: 2-[[2-tert-butoxycarbonyl-4-chloro-5-[3-[[1-[[3-[3-[4-[[2-(2,6-dioxo-3-piperidyl)-1-oxo-isoindolin-4-yl]amino]-1-piperidyl]propanoylamino]phenyl]methylsulfonyl]-4-piperidyl]amino]phenyl]-3-thienyl]oxy]acetic acid (C-29)

Into a 10 mL single neck round bottom flask containing a well-stirred solution of 2-[[2-tert-butoxycarbonyl-4-chloro-5-[3-[[1-[[3-[3-(4-oxo-1-piperidyl)propanoylamino]phenyl]methylsulfonyl]-4-piperidyl]amino]phenyl]-3-thienyl]oxy]acetic acid (A-70, 70 mg, 77.69 μmol, TFA salt) and 3-(4-amino-1-oxo-isoindolin-2-yl)piperidine-2,6-dione (B-63, 34.49 mg, 133.02 μmol) in methanol (2 mL) was added acetic acid (53.25 mg, 886.80 μmol, 50.72 μL) dropwise at 0° C. and stirred for 5 min. Then 2-picoline borane complex (14.23 mg, 133.02 μmol) was added portion wise. The reaction mixture was stirred at room temperature for 30 h. The reaction mixture was cooled to 0° C., quenched with water (0.5 mL) and concentrated under reduced pressure. The residue was purified by reverse phase preparative HPLC (Column: SunFire C18-19×150 mm; Mobile phase A-0.1% TFA in water; Mobile phase B-MeCN) to afford 2-[[2-tert-butoxycarbonyl-4-chloro-5-[3-[[1-[[3-[3-[4-[[2-(2,6-dioxo-3-piperidyl)-1-oxo-isoindolin-4-yl]amino]-1-piperidyl]propanoylamino]phenyl]methylsulfonyl]-4-piperidyl]amino]phenyl]-3-thienyl]oxy]acetic acid (C-29, 30 mg, 24.74 μmol, 32% yield, TFA salt) as an off white solid. LCMS (ES+): m/z 1031.7 [M+H]⁺.

### Step 2: 3-(carboxymethoxy)-4-chloro-5-[3-[[1-[[3-[3-[4-[[2-(2,6-dioxo-3-piperidyl)-1-oxo-isoindolin-4-yl]amino]-1-piperidyl]propanoylamino]phenyl]methylsulfonyl]-4-piperidyl]amino]phenyl]thiophene-2-carboxylic acid (50)

3-(carboxymethoxy)-4-chloro-5-[3-[[1-[[3-[3-[4-[[2-(2,6-dioxo-3-piperidyl)-1-oxo-isoindolin-4-yl]amino]-1-piperidyl]propanoylamino]phenyl]methylsulfonyl]-4-piperidyl]amino]phenyl]thiophene-2-carboxylic acid (50, 5.6 mg, 5.64 μmol, 23% yield) was synthesized from 2-[[2-tert-butoxycarbonyl-4-chloro-5-[3-[[1-[[3-[3-[4-[[2-(2,6-dioxo-3-piperidyl)-1-oxo-isoindolin-4-yl]amino]-1-piperidyl]propanoylamino]phenyl]methylsulfonyl]-4-piperidyl]amino]phenyl]-3-thienyl]oxy]acetic acid (C-29) in a similar fashion to Compound C-1, except using 28 eq. of TFA. The residue was purified by reverse phase prep HPLC (Column: SunFire 18-OBD, waters; Mobile phase A-0.1% TFA in water; Mobile phase B-MeCN). LCMS (ES+): m/z 975.6 [M+H]⁺. ¹H NMR (400 MHz, DMSO-d₆) δ 11.04 (s, 1H), 10.28 (s, 1H), 7.66 (d, J=8.4 Hz, 1H), 7.60 (s, 1H), 7.37-7.28 (m, 2H), 7.19 (t, J=8.0 Hz, 1H), 7.12 (d, J=7.7 Hz, 1H), 6.98 (d, J=7.4 Hz, 1H), 6.87 (d, J=8.2 Hz, 1H), 6.84-6.79 (m, 2H), 6.70-6.65 (m, 1H), 5.88 (d, J=8.2 Hz, 1H), 5.60-5.52 (m, 1H), 5.14 (dd, J=13.3, 5.1 Hz, 1H), 4.80 (s, 2H), 4.40 (s, 2H), 4.25 (d, J=17.3 Hz, 1H), 4.15 (d, J=17.2 Hz, 1H), 3.73-3.65 (m, 1H), 3.60-3.51 (m, 3H), 3.21-3.09 (m, 3H), 2.99-2.84 (m, 6H), 2.66-2.56 (m, 2H), 2.31-2.22 (m, 1H), 2.19-2.09 (m, 2H), 2.08-2.01 (m, 1H), 1.95 (d, J=12.0 Hz, 2H), 1.86-1.64 (m, 2H), 1.43-1.28 (m, 2H).

### Example 51

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]phenyl]thiophene-2-carboxylic acid (51)

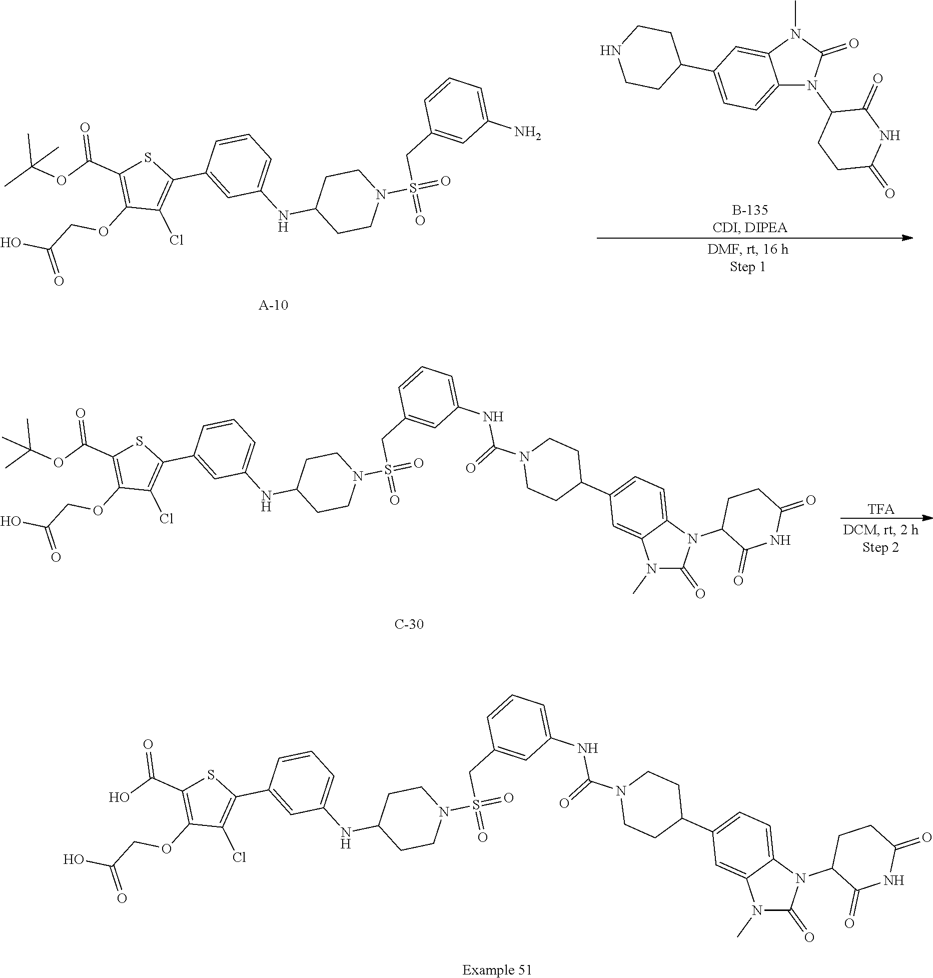

### Step 1: 2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]phenyl]-3-thienyl]oxy]acetic acid (C-30)

2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]phenyl]-3-thienyl]oxy]acetic acid (C-30, 80 mg, 57.34 μmol, 23% yield) was synthesized from 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) and 3-[3-methyl-2-oxo-5-(4-piperidyl)benzimidazol-1-yl]piperidine-2,6-dione (B-135) in a similar fashion to Example 23, except using 1:1 THF:DMF as the solvent. The residue was purified by reverse phase preparative HPLC Purification method: SunFire Prepc18 OBD 5 μm (19×150 mm); Mobile phase A: 0.1% TFA in water and Mobile phase B: MeCN)]. LCMS (ES+): m/z 1004.2 [M+H]⁺.

### Step 2: 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]phenyl]thiophene-2-carboxylic acid (51)

3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]phenyl]thiophene-2-carboxylic acid (51, 29.56 mg, 27.7 μmol, 40% yield, TFA salt) was synthesized from 2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]phenyl]-3-thienyl]oxy]acetic acid (C-30) in a similar fashion to Compound C-1, except using 25 eq. of TFA. The residue was purified using reverse phase preparative HPLC (Purification method: Column: SunFire DC 18-19×150 mm-5 μm; Mobile phase A: 0.1% TFA in water and Mobile phase B: MeCN). LCMS (ES+): m/z 949.0 [M+H]⁺. ¹H NMR (400 MHz, DMSO-d6): 13.16 (bs, 2H), 11.09 (s, 1H), 8.63 (s, 1H), 7.60 (s, 1H), 7.44 (d, J=7.88 Hz, 1H), 7.27-7.18 (m, 2H), 7.13 (s, 1H), 7.03-6.99 (m, 2H), 6.94 (d, J=8.00 Hz, 1H), 6.88 (s, 1H), 6.81 (d, J=8.00 Hz, 1H), 6.70 (d, J=8.00 Hz, 1H), 5.61-5.54 (m, 1H), 5.38-5.31 (m, 1H), 4.92 (s, 2H), 4.35-4.31 (m, 4H), 3.85-3.75 (m, 4H), 3.54 (d, J=12.44 Hz, 2H), 3.50-3.40 (m, 1H), 3.34 (s, 3H), 2.98-2.90 (m, 2H), 2.88-2.81 (m, 2H), 2.71-2.78 (m, 1H), 1.62-2.01 (m, 8H), 1.51-1.33 (m, 2H).

### Example 52

### 3-(carboxymethoxy)-4-chloro-5-[3-[[1-[[3-[[4-[1-(2,6-dioxo-3-piperidyl)-3-methyl-2-oxo-benzimidazol-4-yl]piperidine-1-carbonyl]amino]phenyl]methylsulfonyl]-4-piperidyl]amino]phenyl]thiophene-2-carboxylic acid (52)

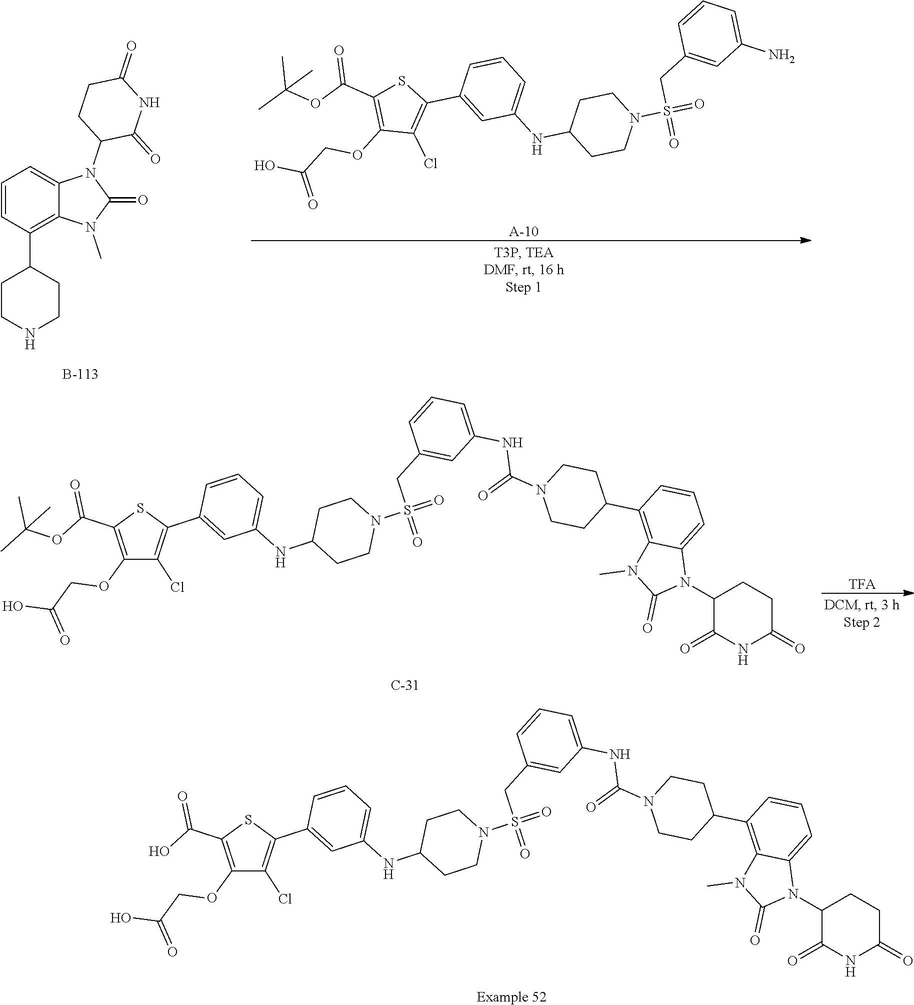

### Step 1: 2-((2-(tert-butoxycarbonyl)-4-chloro-5-(3-((1-((3-(4-(1-(2,6-dioxopiperidin-3-yl)-3-methyl-2-oxo-2,3-dihydro-1H-benzo[d]imidazol-4-yl)piperidine-1-carboxamido)benzyl)sulfonyl)piperidin-4-yl)amino)phenyl)thiophen-3-yl)oxy)acetic acid (C-31)

2-((2-(tert-butoxycarbonyl)-4-chloro-5-(3-((1-((3-(4-(1-(2,6-dioxopiperidin-3-yl)-3-methyl-2-oxo-2,3-dihydro-1H-benzo[d]imidazol-4-yl)piperidine-1-carboxamido)benzyl)sulfonyl)piperidin-4-yl)amino)phenyl)thiophen-3-yl)oxy)acetic acid (C-31, 13 mg, 12.16 μmol, 8% yield) was synthesized from 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) and 3-[3-methyl-2-oxo-4-(4-piperidyl)benzimidazol-1-yl]piperidine-2,6-dione (B-113) in a similar fashion to Example 23, except using excess DIPEA as base and DCM as solvent. The product was purified by reverse phase prep HPLC. LCMS (ES+): m/z 1005.5 [M+H]⁺.

### Step 2: 3-(carboxymethoxy)-4-chloro-5-[3-[[1-[[3-[[4-[1-(2,6-dioxo-3-piperidyl)-3-methyl-2-oxo-benzimidazol-4-yl]piperidine-1-carbonyl]amino]phenyl]methylsulfonyl]-4-piperidyl]amino]phenyl]thiophene-2-carboxylic acid (52)

3-(carboxymethoxy)-4-chloro-5-[3-[[1-[[3-[[4-[1-(2,6-dioxo-3-piperidyl)-3-methyl-2-oxo-benzimidazol-4-yl]piperidine-1-carbonyl]amino]phenyl]methylsulfonyl]-4-piperidyl]amino]phenyl]thiophene-2-carboxylic acid (52, 18.5 mg, 18.98 μmol, 76% yield, TFA salt) was synthesized from 2-((2-(tert-butoxycarbonyl)-4-chloro-5-(3-((1-((3-(4-(1-(2,6-dioxopiperidin-3-yl)-3-methyl-2-oxo-2,3-dihydro-1H-benzo[d]imidazol-4-yl)piperidine-1-carboxamido)benzyl)sulfonyl)piperidin-4-yl)amino)phenyl)thiophen-3-yl)oxy)acetic acid (C-31) in a similar fashion to Compound C-1, except using 25 eq. of TFA. Product isolated by trituration from diethyl ether. LCMS (ES+): m/z 948.2 [M+H]⁺. ¹H NMR (400 MHz, DMSO-d₆) δ 11.10 (s, 1H), 8.65 (s, 1H), 7.60 (s, 1H), 7.43 (d, J=8.2 Hz, 1H), 7.30-7.15 (m, 2H), 7.05-6.94 (m, 4H), 6.87 (s, 1H), 6.81 (d, J=7.5 Hz, 1H), 6.70 (d, J=8.2 Hz, 1H), 5.89 (s, 1H), 5.38 (dd, J=12.6, 5.4 Hz, 1H), 4.91 (s, 2H), 4.40-4.25 (m, 4H), 3.63 (s, 3H), 3.57-3.49 (m, 3H), 3.03-2.83 (m, 6H), 2.74-2.60 (m, 3H), 2.05-1.91 (m, 3H), 1.89-1.82 (m, 2H), 1.74-1.63 (m, 2H), 1.42-1.30 (m, 2H).

### Example 53

### 3-(carboxymethoxy)-4-chloro-5-[3-[[1-[[3-[3-[[1-(2,6-dioxo-3-piperidyl)-3-methyl-2-oxo-benzimidazol-4-yl]propanoylamino]phenyl]methylsulfonyl]-4-piperidyl]amino]phenyl]thiophene-2-carboxylic acid (53)

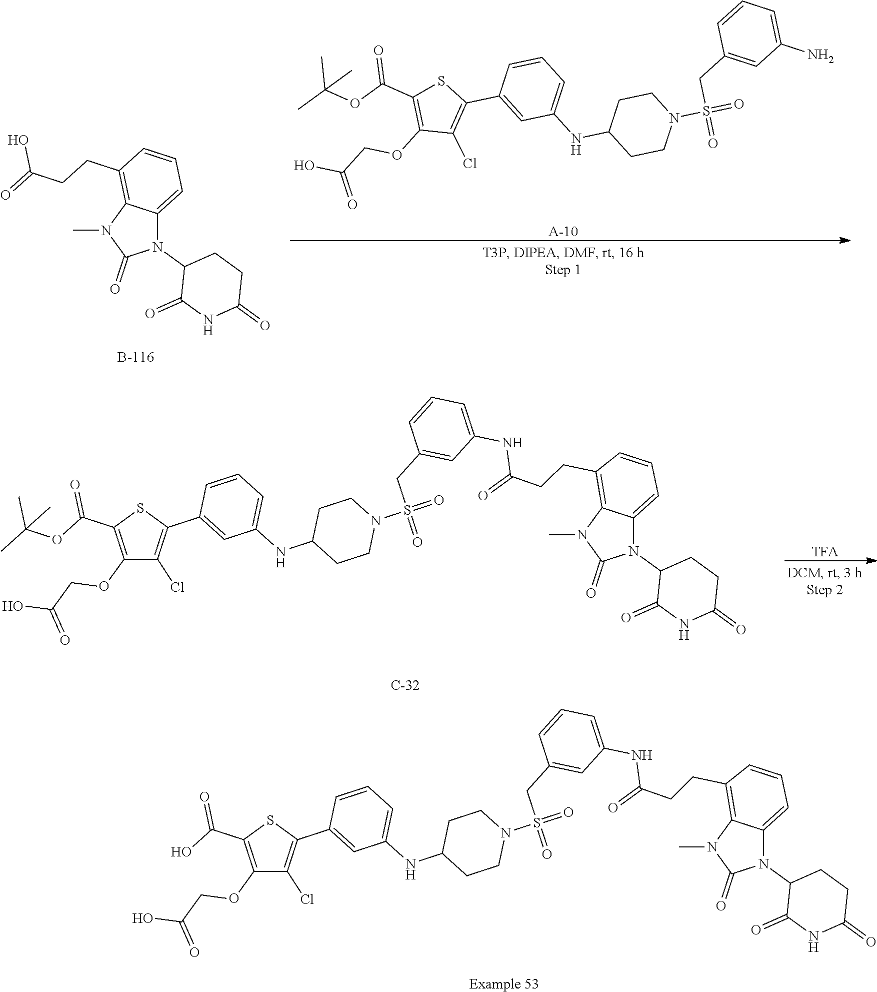

### Step 1: 2-[[2-tert-butoxycarbonyl-4-chloro-5-[3-[[1-[[3-[3-[[1-(2,6-dioxo-3-piperidyl)-3-methyl-2-oxo-benzimidazol-4-yl]propanoylamino]phenyl]methylsulfonyl]-4-piperidyl]amino]phenyl]-3-thienyl]oxy]acetic acid (C-32)

2-[[2-tert-butoxycarbonyl-4-chloro-5-[3-[[1-[[3-[3-[[1-(2,6-dioxo-3-piperidyl)-3-methyl-2-oxo-benzimidazol-4-yl]propanoylamino]phenyl]methylsulfonyl]-4-piperidyl]amino]phenyl]-3-thienyl]oxy]acetic acid (C-32, 18 mg, 11.28 μmol, 5% yield) was synthesized from 3-[1-(2,6-dioxo-3-piperidyl)-3-methyl-2-oxo-benzimidazol-4-yl]propanoic acid (B-116) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Compound C-6, except using DIPEA as base. The residue was purified by reverse phase prep HPLC (Column: SunFire-C18, 100 mm; Mobile phase A: 0.1% TFA in Water, Mobile phase B: MeCN). LCMS (ES+): m/z 949.1 [M+H]⁺.

### Step 2: 3-(carboxymethoxy)-4-chloro-5-[3-[[1-[[3-[3-[[1-(2,6-dioxo-3-piperidyl)-3-methyl-2-oxo-benzimidazol-4-yl]propanoylamino]phenyl]methylsulfonyl]-4-piperidyl]amino]phenyl]thiophene-2-carboxylic acid (53)

3-(carboxymethoxy)-4-chloro-5-[3-[[1-[[3-[3-[[1-(2,6-dioxo-3-piperidyl)-3-methyl-2-oxo-benzimidazol-4-yl]propanoylamino]phenyl]methylsulfonyl]-4-piperidyl]amino]phenyl]thiophene-2-carboxylic acid (53, 4.80 mg, 5.35 μmol, 28% yield) was synthesized from 2-[[2-tert-butoxycarbonyl-4-chloro-5-[3-[[1-[[3-[3-[1-(2,6-dioxo-3-piperidyl)-3-methyl-2-oxo-benzimidazol-4-yl]propanoylamino]phenyl]methylsulfonyl]-4-piperidyl]amino]phenyl]-3-thienyl]oxy]acetic acid (C-32) in a similar fashion to Compound C-1, except using 150 eq. of TFA. The residue was purified by reverse phase prep HPLC (Column: SunFire-C18 100 mm; Mobile phase A: 0.1% TFA in Water, Mobile phase B: MeCN). LCMS (ES+): m/z 892.6 [M+H]⁺. ¹H NMR (400 MHz, DMSO-d6): δ 11.10 (s, 1H), 10.04 (s, 1H), 7.66 (s, 1H), 7.57-7.58 (m, 1H), 7.29-7.31 (m, 1H), 7.19-7.21 (m, 1H), 7.08-7.10 (m, 1H), 6.90-6.91 (m, 4H), 6.81-6.83 (m, 1H), 6.73 (s, 1H), 5.36-5.38 (m, 1H), 4.93 (s, 2H), 4.37 (s, 2H), 3.63 (s, 3H), 3.26-3.28 (m, 3H), 2.94-2.96 (m, 3H), 2.60-2.64 (m, 3H), 1.98-1.80 (m, 1H), 1.37-1.39 (m, 2H).

### Example 54

### 3-(carboxymethoxy)-4-chloro-5-[3-[[1-[[3-[2-[4-[3-(2,6-dioxo-3-piperidyl) indazol-1-yl]-1-piperidyl]acetyl]amino]phenyl]methylsulfonyl]-4-piperidyl]amino]phenyl]thiophene-2-carboxylic acid (54)

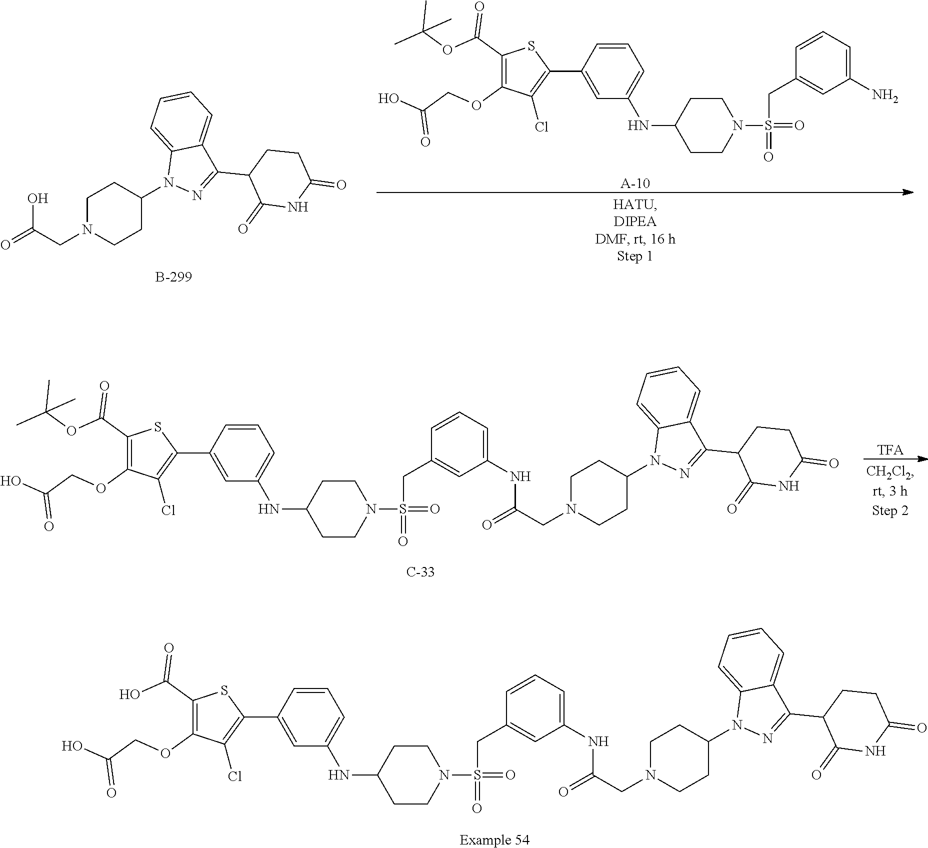

### Step 1: 2-[[2-tert-butoxycarbonyl-4-chloro-5-[3-[[1-[[3-[2-[4-[3-(2,6-dioxo-3-piperidyl) indazol-1-yl]-1-piperidyl]acetyl]amino]phenyl]methylsulfonyl]-4-piperidyl]amino]phenyl]-3-thienyl]oxy]acetic acid (C-33)

2-[[2-tert-butoxycarbonyl-4-chloro-5-[3-[[1-[[3-[[2-[4-[3-(2,6-dioxo-3-piperidyl) indazol-1-yl]-1-piperidyl]acetyl]amino]phenyl]methylsulfonyl]-4-piperidyl]amino]phenyl]-3-thienyl]oxy]acetic acid (C-33, 20 mg, 17.80 μmol, 9% yield) was synthesized from 2-[4-[3-(2,6-dioxo-3-piperidyl) indazol-1-yl]-1-piperidyl]acetic acid (B-299 and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 1, except using DMF as the solvent. The residue was subjected to reverse phase prep HPLC [Column: XBridge C18, 150 mm, Mobile phase: 0.1% TFA in water/MeCN]. LCMS (ES+): m/z 987.7 [M+H]⁺.

### Step 2: 3-(carboxymethoxy)-4-chloro-5-[3-[[1-[[3-[[2-[4-[3-(2,6-dioxo-3-piperidyl) indazol-1-yl]-1-piperidyl]acetyl]amino]phenyl]methylsulfonyl]-4-piperidyl]amino]phenyl]thiophene-2-carboxylic acid (54)

3-(carboxymethoxy)-4-chloro-5-[3-[[1-[[3-[[2-[4-[3-(2,6-dioxo-3-piperidyl) indazol-1-yl]-1-piperidyl]acetyl]amino]phenyl]methylsulfonyl]-4-piperidyl]amino]phenyl]thiophene-2-carboxylic acid (54, 10 mg, 10.13 μmol, 50% yield) was synthesized from 2-[2-tert-butoxycarbonyl-4-chloro-5-[3-[[1-[[3-[[2-[4-[3-(2,6-dioxo-3-piperidyl) indazol-1-yl]-1-piperidyl]acetyl]amino]phenyl]methylsulfonyl]-4-piperidyl]amino]phenyl]-3-thienyl]oxy]acetic acid (C-33) in a similar fashion to Compound C-1, except using 5 eq. of TFA. Product isolated by trituration from diethyl ether. LCMS (ES+): m/z 932.2 [M+H]⁺. ¹H NMR (400 MHz, DMSO-d6): δ 7.70 (d, J=8.00 Hz, 1H), 7.64 (d, J=8.40 Hz, 1H), 7.59 (s, 2H), 7.39-7.23 (m, 3H), 7.16-7.02 (m, 5H), 6.81 (s, 1H), 6.74 (d, J=7.20 Hz, 1H), 6.63 (d, J=7.60 Hz, 1H), 5.84 (br s, 1H), 4.85 (s, 3H), 4.36-4.34 (m, 3H), 4.25-4.10 (m, 2H), 3.70-3.60 (m, 2H), 3.55-3.45 (m, 6H), 2.95-2.86 (m, 2H), 2.20-2.05 (m, 4H), 1.91-1.89 (m, 3H), 1.32-1.29 (m, 2H), 1.22-1.10 (m, 2H).

### Example 55

### 3-(carboxymethoxy)-4-chloro-5-[3-[[1-[[3-[[2-[4-[1-(2,6-dioxo-3-piperidyl)-3-methyl-2-oxo-benzimidazol-5-yl]-1-piperidyl]acetyl]amino]phenyl]methylsulfonyl]-4-piperidyl]amino]phenyl]thiophene-2-carboxylic acid (55)

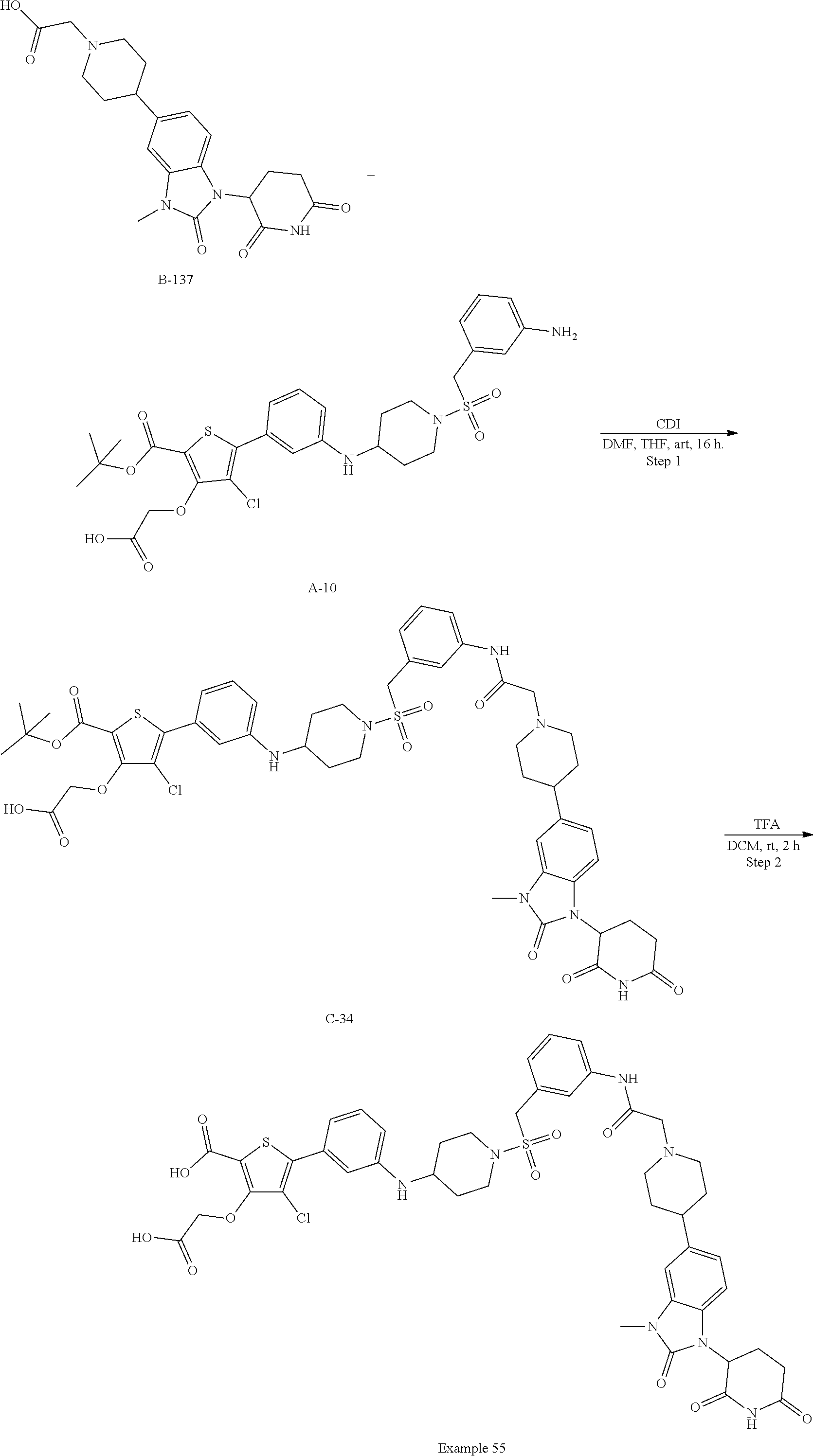

### Step 1: 2-[[2-tert-butoxycarbonyl-4-chloro-5-[3-[[1-∥3-[[2-[4-[1-(2,6-dioxo-3-piperidyl)-3-methyl-2-oxo-benzimidazol-5-yl]-1-piperidyl]acetyl]amino]phenyl]methylsulfonyl]-4-piperidyl]amino]phenyl]-3-thienyl]oxy]acetic acid (C-34)

2-[[2-tert-butoxycarbonyl-4-chloro-5-[3-[[1-[[3-[[2-[4-[1-(2,6-dioxo-3-piperidyl)-3-methyl-2-oxo-benzimidazol-5-yl]-1-piperidyl]acetyl]amino]phenyl]methylsulfonyl]-4-piperidyl]amino]phenyl]-3-thienyl]oxy]acetic acid (C-34, 8.5 mg, 8.09 μmol, 8% yield, Formic acid salt) was synthesized from 2-[4-[1-(2,6-dioxo-3-piperidyl)-3-methyl-2-oxo-benzimidazol-5-yl]-1-piperidyl]acetic acid (B-137) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Example 23, except using 1:1 DMF:THF as the solvent. The residue was purified by reverse phase prep HPLC (Column: XBridge C18, 19×150 mm, Sum; Mobile phase A-0.1% formic acid in water; Mobile phase B-MeCN). LCMS (ES+): m/z 1018.2 [M+H]⁺.

### Step 2: 3-(carboxymethoxy)-4-chloro-5-[3-[[1-[[3-[2-[4-[1-(2,6-dioxo-3-piperidyl)-3-methyl-2-oxo-benzimidazol-5-yl]-1-piperidyl]acetyl]amino]phenyl]methylsulfonyl]-4-piperidyl]amino]phenyl]thiophene-2-carboxylic acid (55)

3-(carboxymethoxy)-4-chloro-5-[3-[[1-[[3-[[2-[4-[1-(2,6-dioxo-3-piperidyl)-3-methyl-2-oxo-benzimidazol-5-yl]-1-piperidyl]acetyl]amino]phenyl]methylsulfonyl]-4-piperidyl]amino]phenyl]thiophene-2-carboxylic acid (55, 5.62 mg, 5.02 μmol, 63% yield, TFA salt) was synthesized from 2-[[2-tert-butoxycarbonyl-4-chloro-5-[3-[[1-[[3-[[2-[4-[1-(2,6-dioxo-3-piperidyl)-3-methyl-2-oxo-benzimidazol-5-yl]-1-piperidyl]acetyl]amino]phenyl]methylsulfonyl]-4-piperidyl]amino]phenyl]-3-thienyl]oxy]acetic acid (C-34) in a similar fashion to Compound C-1, except using 10 eq. of TFA. Product isolated by trituration from diethyl ether. LCMS (ES+): m/z 961.6 [M+H]⁺. ¹H NMR (400 MHz, DMSO-d₆) δ 11.10 (s, 1H), 10.53 (s, 1H), 7.66 (s, 1H), 7.64 (s, 1H), 7.39 (t, J=7.8 Hz, 1H), 7.23-7.16 (m, 2H), 7.10-7.04 (m, 2H), 6.93 (d, J=8.2 Hz, 1H), 6.87-6.83 (m, 1H), 6.81 (d, J=7.6 Hz, 1H), 6.72-6.67 (m, 1H), 5.88 (d, J=7.9 Hz, 1H), 5.36 (dd, J=12.7, 5.4 Hz, 1H), 4.82 (s, 2H), 4.42 (s, 2H), 4.05 (s, 2H), 3.58-3.52 (m, 4H), 3.00-2.89 (m, 4H), 2.89-2.78 (m, 2H), 2.76-2.59 (m, 4H), 2.08-1.92 (m, 8H), 1.43-1.30 (m, 3H), 1.28-1.20 (m, 2H).

### Example 56

### 3-(carboxymethoxy)-4-chloro-5-[3-[[(4S)-1-[[3-[3-[4-[[2-(2,6-dioxo-3-piperidyl)-1-oxo-isoindolin-4-yl]oxy-1-piperidyl]propanoylamino]phenyl]methylsulfonyl]-2,2-dimethyl-4-piperidyl]amino]phenyl]thiophene-2-carboxylic acid (56)

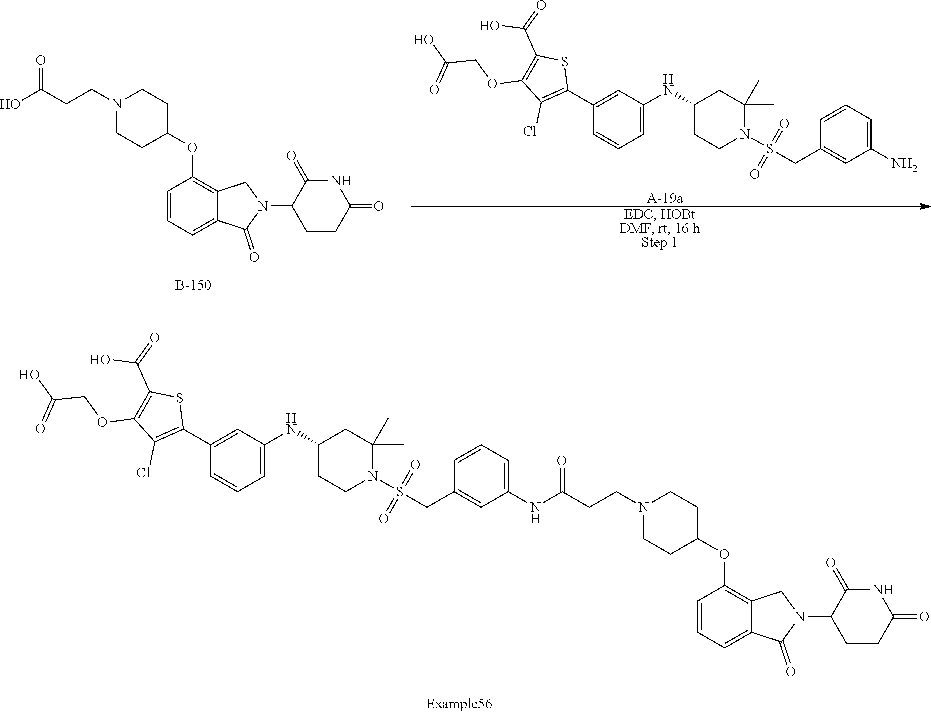 [SMILES: O=C(O)CCN1CCC(Oc2cccc3c2CN(C2CCC(=O)NC2=O)C3=O)CC1 ; CC1(C)C[C@@H](Nc2cccc(-c3sc(C(=O)O)c(OCC(=O)O)c3Cl)c2)CCN1S(=O)(=O)Cc1cccc(N)c1 ; "EDC, HOBtDMF, rt, 16 hStep 1" ; "A-19a" ; "B-150" ; CC1(C)C[C@@H](Nc2cccc(-c3sc(C(=O)O)c(OCC(=O)O)c3Cl)c2)CCN1S(=O)(=O)Cc1cccc(NC(=O)CCN2CCC(Oc3cccc4c3CN(C3CCC(=O)NC3=O)C4=O)CC2)c1]

### Step 1: 3-(carboxymethoxy)-4-chloro-5-[3-[[(4S)-1-[[3-[3-[4-[[2-(2,6-dioxo-3-piperidyl)-1-oxo-isoindolin-4-yl]oxy-1-piperidyl]propanoylamino]phenyl]methylsulfonyl]-2,2-dimethyl-4-piperidyl]amino]phenyl]thiophene-2-carboxylic acid (56)

3-(carboxymethoxy)-4-chloro-5-[3-[[(4S)-1-[[3-[3-[4-[[2-(2,6-dioxo-3-piperidyl)-1-oxo-isoindolin-4-yl]oxy-1-piperidyl]propanoylamino]phenyl]methylsulfonyl]-2,2-dimethyl-4-piperidyl]amino]phenyl]thiophene-2-carboxylic acid (56, 11 mg, 9.66 μmol, 6% yield, TFA salt) was synthesized from 3-(4-((2-(2,6-dioxopiperidin-3-yl)-1-oxoisoindolin-4-yl)oxy)piperidin-1-yl)propanoic acid (B-150) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 46. The residue was purified by reverse phase preparative HPLC (Column: SunFire-C18-OBD (Waters); Mobile phase: 0.1% TFA in water/MeCN). LCMS (ES+): m/z 1005.1 [M+H]⁺. ¹H NMR (400 MHz, DMSO-d6): δ 11.01 (s, 1H), 10.25 (s, 1H), 9.30 (s, 1H), 7.62-7.64 (m, 2H), 7.47-7.49 (m, 1H), 7.31-7.32 (m, 3H), 7.19 (t, J=8.00 Hz, 1H), 7.11 (d, J=8.00 Hz, 1H), 6.87 (s, 1H), 6.80 (d, J=7.60 Hz, 1H), 6.69 (d, J=6.80 Hz, 1H), 5.80 (s, 1H), 5.12-5.15 (m, 1H), 4.91 (s, 2H), 4.19-4.24 (m, 4H), 3.72 (s, 2H), 3.09-3.12 (m, 3H), 2.87-2.99 (m, 3H), 2.03-2.06 (m, 4H), 1.77-1.80 (m, 4H), 1.23-1.34 (m, 9H), 1.08-1.10 (m, 1H).

### Example 57

### 3-(carboxymethoxy)-4-chloro-5-[3-[[(4S)-1-[[3-[[1-[2-[[2-(2,6-dioxo-3-piperidyl)-1-oxo-isoindolin-5-yl]amino]acetyl]azetidine-3-carbonyl]amino]phenyl]methylsulfonyl]-2,2-dimethyl-4-piperidyl]amino]phenyl]thiophene-2-carboxylic acid (57)

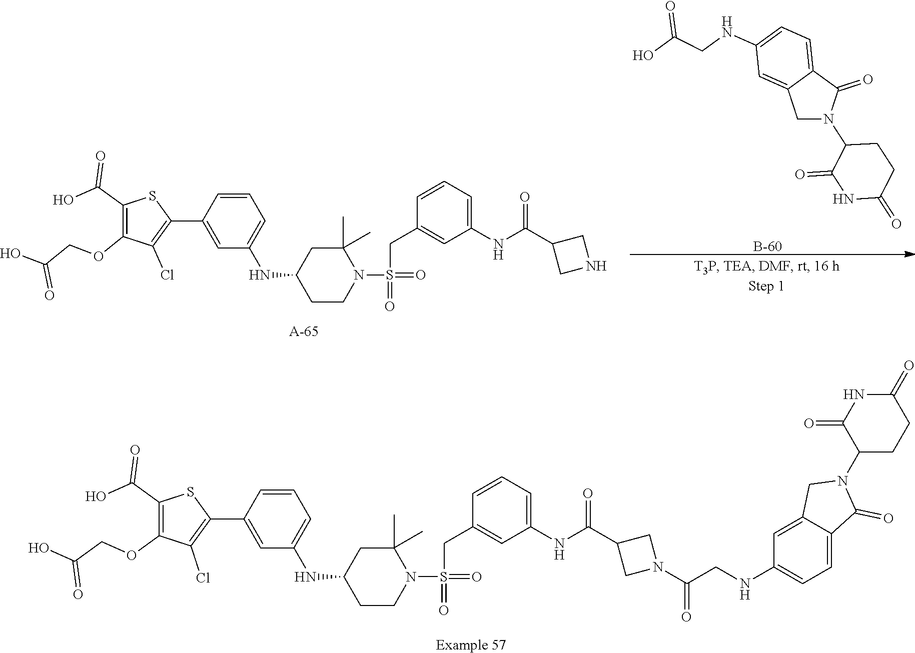

### Step 1: 3-(carboxymethoxy)-4-chloro-5-[3-[[(4S)-1-[[3-[[1-[2-[[2-(2,6-dioxo-3-piperidyl)-1-oxo-isoindolin-5-yl]amino]acetyl]azetidine-3-carbonyl]amino]phenyl]methylsulfonyl]-2,2-dimethyl-4-piperidyl]amino]phenyl]thiophene-2-carboxylic acid (57)

3-(carboxymethoxy)-4-chloro-5-[3-[[(4S)-1-[3-[[1-[2-[[2-(2,6-dioxo-3-piperidyl)-1-oxo-isoindolin-5-yl]amino]acetyl]azetidine-3-carbonyl]amino]phenyl]methylsulfonyl]-2,2-dimethyl-4-piperidyl]amino]phenyl]thiophene-2-carboxylic acid (57, 7 mg, 5.79 μmol, 15% yield, TFA salt) was synthesized from 2-[[2-(2,6-dioxo-3-piperidyl)-1-oxo-isoindolin-5-yl]amino]acetic acid (B-60) and 5-[3-[(4S)-1-[[3-(azetidine-3-carbonylamino)phenyl]methylsulfonyl]-2,2-dimethyl-4-piperidyl]amino]phenyl]-3-(carboxymethoxy)-4-chloro-thiophene-2-carboxylic acid (A-65) in a similar fashion to Compound C-6. The residue was purified using reverse phase prep HPLC [Purification method: Column: SunFire C18 (19×150 mm) 5 μm; Mobile phase A: 0.1% TFA in water; Mobile phase B: MeCN; Flow Rate: 15.0 mL/min]. LCMS (ES+): m/z 990.49 [M+H]⁺. ¹H NMR (400 MHz, DMSO-d₆) δ 10.93 (s, 1H), 10.17 (s, 1H), 7.67 (s, 1H), 7.63 (d, J=8.3 Hz, 1H), 7.41 (d, J=8.3 Hz, 1H), 7.33 (t, J=7.8 Hz, 1H), 7.25-7.15 (m, 1H), 7.13-7.07 (m, 1H), 6.86 (s, 1H), 6.80 (d, J=7.6 Hz, 1H), 6.74-6.61 (m, 3H), 5.01 (dd, J=13.0, 4.9 Hz, 1H), 4.91 (s, 2H), 4.43-4.19 (m, 6H), 4.14 (d, J=16.3 Hz, 1H), 4.10-3.95 (m, 3H), 3.81 (s, 2H), 3.17-3.08 (m, 2H), 2.95-2.82 (m, 3H), 2.62-2.58 (m, 1H), 1.99-1.84 (m, 2H), 1.82-1.73 (m, 1H), 1.46 (s, 3H), 1.42-1.35 (m, 4H), 1.27-1.20 (m, 1H), 1.16-1.03 (m, 1H).

### Example 58

### 3-(carboxymethoxy)-4-chloro-5-[3-[[(4S)-1-[3-[[1-[2-[[2-(2,6-dioxo-3-piperidyl)-1,3-dioxo-isoindolin-4-yl]oxyacetyl]piperidine-4-carbonyl]amino]phenyl]methylsulfonyl]-2,2-dimethyl-4-piperidyl]amino]phenyl]thiophene-2-carboxylic acid (58)

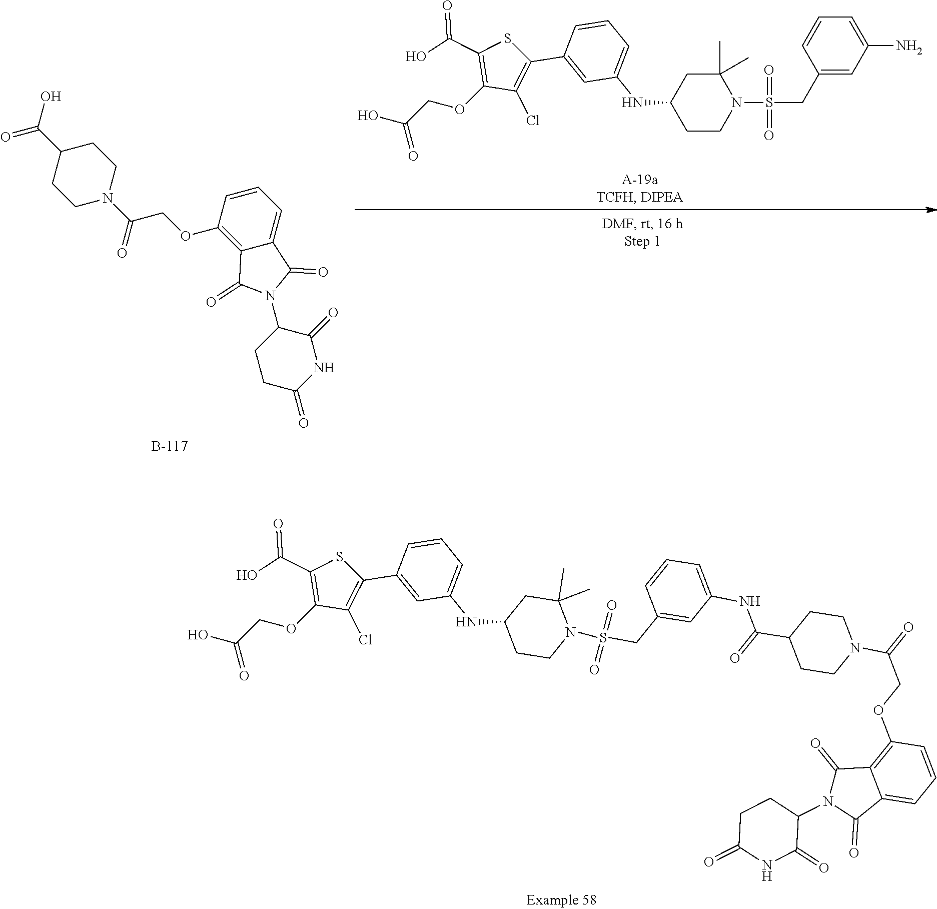 [SMILES: O=C1CCC(N2C(=O)c3cccc(OCC(=O)N4CCC(C(=O)O)CC4)c3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)COc3cccc4c3C(=O)N(C3CCC(=O)NC3=O)C4=O)CC2)c1]

### Step 1: 3-(carboxymethoxy)-4-chloro-5-[3-[[(4S)-1-[3-[[1-[2-[[2-(2,6-dioxo-3-piperidyl)-1,3-dioxo-isoindolin-4-yl]oxyacetyl]piperidine-4-carbonyl]amino]phenyl]methylsulfonyl]-2,2-dimethyl-4-piperidyl]amino]phenyl]thiophene-2-carboxylic acid (58)

Into a 10 mL single neck round bottom flask containing a well-stirred solution of 1-[2-[[2-(2,6-dioxo-3-piperidyl)-1,3-dioxo-isoindolin-4-yl]oxyacetyl]piperidine-4-carboxylic acid (B-117, 27.34 mg, 61.66 μmol) in anhydrous DMF (0.5 mL) were added N,N,N′,N′-tetramethylchloroformamidinium-hexafluorophosphate (60 mg, 213.84 μmol) and DIPEA (23.91 mg, 184.99 μmol, 32.22 μL). After 4 h, a solution of 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 30 mg, 49.33 μmol) in anhydrous DMF (0.5 mL) was added. The resulting mixture was stirred at room temperature for 12 h. The reaction was quenched with water (0.5 mL) and volatiles were removed under reduced pressure. The residue was purified by reverse phase prep HPLC [Method: Column-SunFire C18 (150×19) mm, 5 μm; Mobile phase A: 0.1% TFA in water and Mobile phase B: MeCN] to afford 3-(carboxymethoxy)-4-chloro-5-[3-[[(4S)-1-[[3-[1-[2-[[2-(2,6-dioxo-3-piperidyl)-1,3-dioxo-isoindolin-4-yl]oxyacetyl]piperidine-4-carbonyl]amino]phenyl]methylsulfonyl]-2,2-dimethyl-4-piperidyl]amino]phenyl]thiophene-2-carboxylic acid (58, 6.3 mg, 5.79 μmol, 9% yield, TFA salt) as an off-white solid. LCMS (ES+): m/z 1032.8 [M+H]⁺. ¹H NMR (400 MHz, DMSO-d₆) δ 11.11 (s, 1H), 10.03 (s, 1H), 7.81-7.74 (m, 1H), 7.69 (s, 1H), 7.60 (d, J=8.2 Hz, 1H), 7.45 (d, J=7.2 Hz, 1H), 7.36-7.28 (m, 2H), 7.20 (t, J=7.9 Hz, 1H), 7.08 (d, J=7.6 Hz, 1H), 6.88-6.85 (m, 1H), 6.80 (d, J=7.5 Hz, 1H), 6.72-6.65 (m, 1H), 5.28-5.15 (m, 2H), 5.10 (dd, J=12.9, 5.4 Hz, 1H), 4.91 (s, 2H), 4.43-4.22 (m, 3H), 3.86 (d, J=13.3 Hz, 1H), 3.15-3.06 (m, 3H), 2.96-2.82 (m, 2H), 2.76-2.56 (m, 4H), 2.10-1.98 (m, 1H), 1.94-1.69 (m, 5H), 1.56-1.43 (m, 4H), 1.39 (s, 3H), 1.23 (s, 1H), 1.18-1.03 (m, 1H).

### Example 59

### 3-(carboxymethoxy)-4-chloro-5-[3-[[1-[[3-[[4-[[2-(2,6-dioxo-3-piperidyl)-1-oxo-isoindolin-4-yl]amino]piperidine-1-carbonyl]amino]phenyl]methylsulfonyl]-4-piperidyl]amino]phenyl]thiophene-2-carboxylic acid (59)

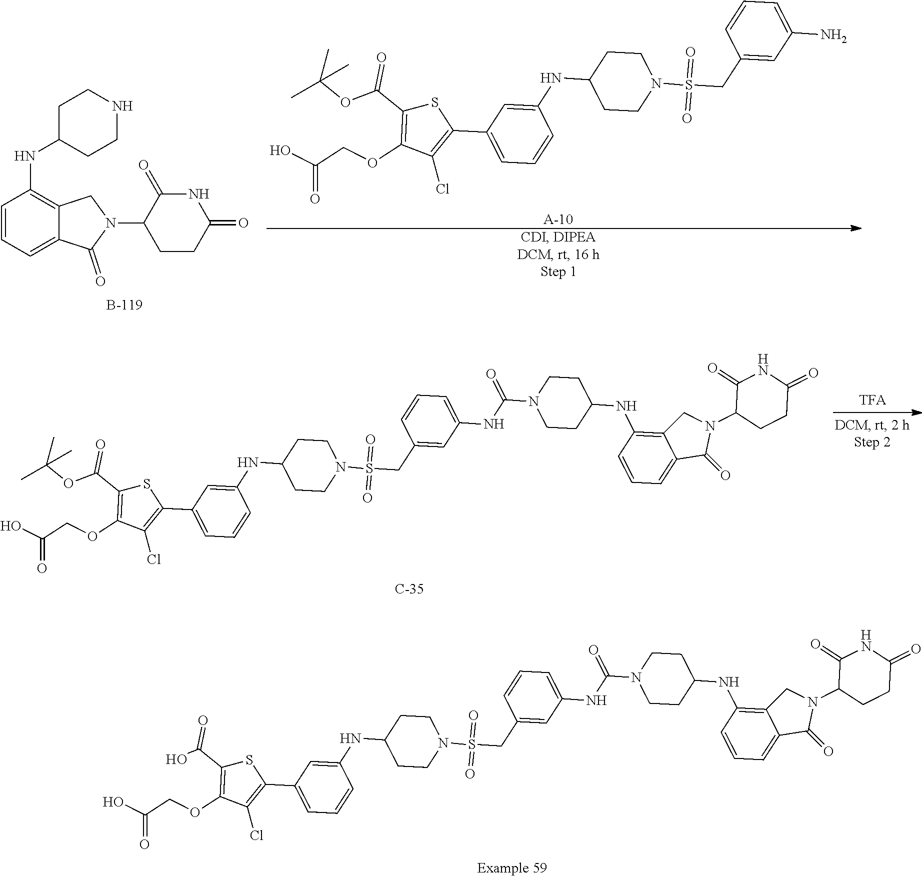

### Step 1: 2-[[2-tert-butoxycarbonyl-4-chloro-5-[3-[[1-[[3-[[4-[[2-(2,6-dioxo-3-piperidyl)-1-oxo-isoindolin-4-yl]amino]piperidine-1-carbonyl]amino]phenyl]methylsulfonyl]-4-piperidyl]amino]phenyl]-3-thienyl]oxy]acetic acid (C-35)

2-[[2-tert-butoxycarbonyl-4-chloro-5-[3-[[1-[[3-[[4-[[2-(2,6-dioxo-3-piperidyl)-1-oxo-isoindolin-4-yl]amino]piperidine-1-carbonyl]amino]phenyl]methylsulfonyl]-4-piperidyl]amino]phenyl]-3-thienyl]oxy]acetic acid (C-35, 40 mg, 35.095 μmol, 28% yield, TFA salt) was synthesized from 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) and 3-[1-oxo-4-(4-piperidyl]amino)isoindolin-2-yl]piperidine-2,6-dione (B-119) in a similar fashion to Example 23, except using excess DIPEA as base and DMF as the solvent. The residue was purified by reverse phase preparative HPLC [Purification method: YMC PRO C18 250×20 mm-5 μm; Mobile phase A: 0.1% TFA in water and Mobile phase B: MeCN). LCMS (ES−): m/z 1002.9 [M−H]⁻.

### Step 2: 3-(carboxymethoxy)-4-chloro-5-[3-[[1-[[3-[[4-[[2-(2,6-dioxo-3-piperidyl)-1-oxo-isoindolin-4-yl]amino]piperidine-1-carbonyl]amino]phenyl]methylsulfonyl]-4-piperidyl]amino]phenyl]thiophene-2-carboxylic acid (59)

3-(carboxymethoxy)-4-chloro-5-[3-[[1-[[3-[[4-[[2-(2,6-dioxo-3-piperidyl)-1-oxo-isoindolin-4-yl]amino]piperidine-1-carbonyl]amino]phenyl]methylsulfonyl]-4-piperidyl]amino]phenyl]thiophene-2-carboxylic acid (59, 23 mg, 20.93 μmol, 63% yield, TFA salt) was synthesized from 2-[[2-tert-butoxycarbonyl-4-chloro-5-[3-[[1-[[3-[[4-[[2-(2,6-dioxo-3-piperidyl)-1-oxo-isoindolin-4-yl]amino]piperidine-1-carbonyl]amino]phenyl]methylsulfonyl]-4-piperidyl]amino]phenyl]-3-thienyl]oxy]acetic acid (C-35) in a similar fashion to Compound C-1, except using 25 eq. of TFA. The product was triturated with diethyl ether. LCMS (ES+): m/z 948.1 [M+H]⁺. ¹H NMR (400 MHz, DMSO-d₆) δ 11.02 (s, 1H), 8.65 (s, 1H), 7.56 (s, 1H), 7.42 (d, J=8.3 Hz, 1H), 7.33-7.16 (m, 3H), 6.99 (d, J=7.6 Hz, 1H), 6.94 (d, J=7.4 Hz, 1H), 6.91-6.85 (m, 2H), 6.81 (d, J=7.6 Hz, 1H), 6.70 (d, J=8.3 Hz, 1H), 5.89 (s, 1H), 5.40 (s, 1H), 5.12 (dd, J=13.4, 5.0 Hz, 1H), 4.92 (s, 2H), 4.33 (s, 2H), 4.23 (d, J=17.3 Hz, 1H), 4.18-4.07 (m, 3H), 3.58-3.48 (m, 2H), 3.05-2.86 (m, 6H), 2.65-2.58 (m, 1H), 2.31-2.19 (m, 2H), 2.06-1.88 (m, 5H), 1.47-1.29 (m, 4H).

### Example 60

### 3-(carboxymethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (60)

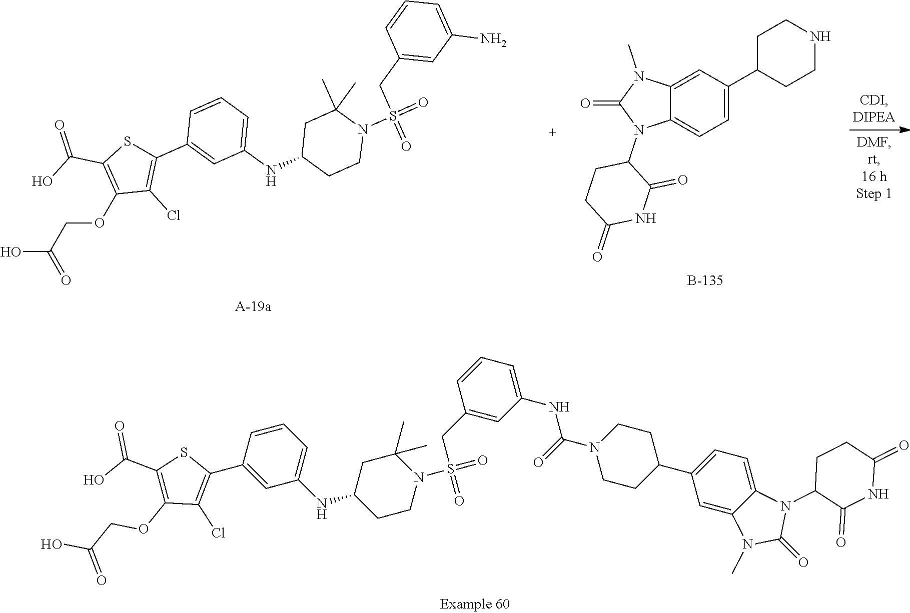 [SMILES: CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (60)

3-(carboxymethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (60, 30 mg, 30.37 μmol, 9% yield, Formic acid salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-methyl-2-oxo-5-(4-piperidyl)benzimidazol-1-yl]piperidine-2,6-dione (B-135) in a similar fashion to Example 23, except using excess DIPEA as base and DMF as the solvent. The residue was purified by reverse phase HPLC (Column: XBridge C18, 19×150 mm, 5 μm; Mobile phase A-0.1% formic acid in water; Mobile phase B-MeCN). LCMS (ES−): m/z 974.2 [M−H]⁻. ¹H NMR (400 MHz, DMSO-d₆) δ 5.66 (d, J=7.8 Hz, 1H), 5.34 (dd, J=12.8, 5.4 Hz, 1H), 4.86 (s, 2H), 4.40-4.21 (m, 4H), 3.57-3.42 (m, 3H), 3.17-3.07 (m, 2H), 2.94-2.84 (m, 3H), 2.82-2.70 (m, 2H), 2.66-2.57 (m, 1H), 2.04-1.96 (m, 1H), 1.89-1.74 (m, 4H), 1.69-1.57 (m, 2H), 1.48 (s, 3H), 1.39 (s, 3H), 1.16-1.03 (m, 1H).

### Example 61

### 3-(carboxymethoxy)-4-chloro-5-(3-(((4S)-1-((3-(1-(3-(2-(2,6-dioxopiperidin-3-yl)-1-oxoisoindolin-4-yl)propanoyl)piperidine-4-carboxamido)benzyl)sulfonyl)-2,2-dimethylpiperidin-4-yl)amino)phenyl)thiophene-2-carboxylic acid (61)

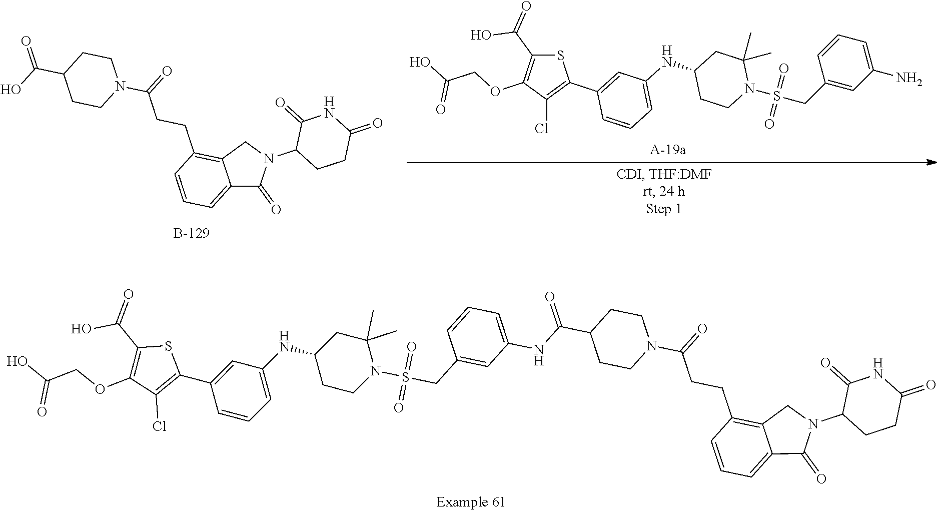 [SMILES: O=C1CCC(N2Cc3c(CCC(=O)N4CCC(C(=O)O)CC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CCc3cccc4c3CN(C3CCC(=O)NC3=O)C4=O)CC2)c1]

### Step 1: 3-(carboxymethoxy)-4-chloro-5-(3-(((4S)-1-((3-(1-(3-(2-(2,6-dioxopiperidin-3-yl)-1-oxoisoindolin-4-yl)propanoyl)piperidine-4-carboxamido)benzyl)sulfonyl)-2,2-dimethylpiperidin-4-yl)amino)phenyl)thiophene-2-carboxylic acid (61)

3-(carboxymethoxy)-4-chloro-5-(3-(((4S)-1-((3-(1-(3-(2-(2,6-dioxopiperidin-3-yl)-1-oxoisoindolin-4-yl)propanoyl)piperidine-4-carboxamido)benzyl)sulfonyl)-2,2-dimethylpiperidin-4-yl)amino)phenyl)thiophene-2-carboxylic acid (61, 8 mg, 6.96 μmol, 6% yield, TFA salt) was synthesized from 1-(3-(2-(2,6-dioxopiperidin-3-yl)-1-oxoisoindolin-4-yl)propanoyl)piperidine-4-carboxylic acid (B-129) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except using 1:1 THF:DMF as the solvent. the residue was purified by reverse phase prep HPLC [Purification method: Column: SunFire C18 (19×150 mm) 5 μm, mobile phase: 0.1% TFA in water and MeCN]. LCMS (ES+): m/z 1015.4 [M−H]⁻. ¹H NMR (400 MHz, DMSO-d₆): δ 11.00 (s, 1H), 10.00 (s, 1H), 7.68-7.44 (m, 4H), 7.30-7.08 (m, 3H), 6.97-6.69 (m, 3H), 5.68 (bs, 1H), 5.12 (dd, J=5.20, 12.80 Hz, 1H), 4.89 (s, 2H), 4.51-4.26 (m, 5H), 3.99-3.90 (m, 2H), 3.15-3.12 (m, 4H), 3.09-2.85 (m, 7H), 2.72-2.68 (m, 3H), 2.07-2.03 (m, 1H), 1.89-1.86 (m, 1H), 1.80-1.77 (m, 3H), 1.47 (s, 3H), 1.39-1.30 (m, 5H), 1.26-1.24 (m, 2H), 1.20-1.11 (m, 2H).

### Example 62

### 3-(carboxymethoxy)-4-chloro-5-(3-(((4S)-1-((3-(4-((2-(2,6-dioxopiperidin-3-yl)-1-oxoisoindolin-4-yl)glycyl) piperazine-1-carboxamido)benzyl)sulfonyl)-2,2-dimethylpiperidin-4-yl)amino)phenyl)thiophene-2-carboxylic acid (62)

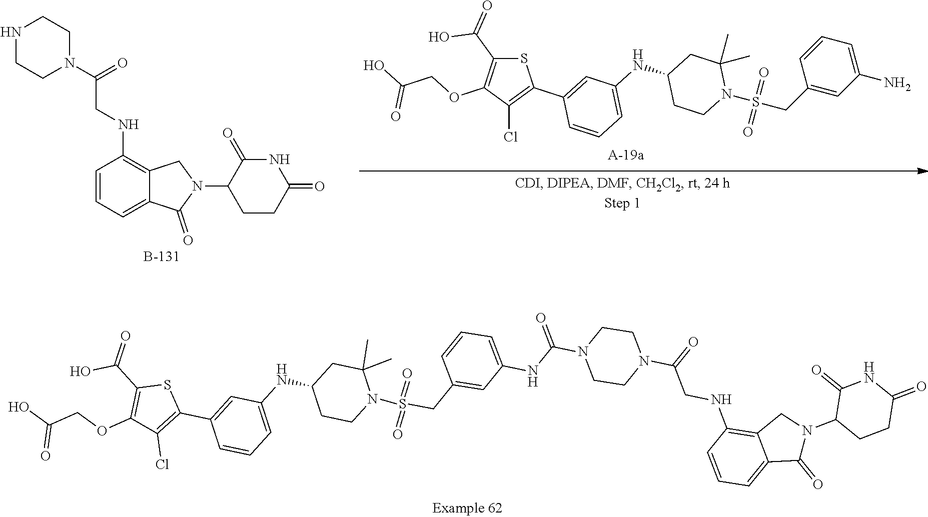 [SMILES: O=C1CCC(N2Cc3c(NCC(=O)N4CCNCC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCN(C(=O)CNc3cccc4c3CN(C3CCC(=O)NC3=O)C4=O)CC2)c1]

### Step 1: 3-(carboxymethoxy)-4-chloro-5-(3-(((4S)-1-((3-(4-((2-(2,6-dioxopiperidin-3-yl)-1-oxoisoindolin-4-yl)glycyl) piperazine-1-carboxamido)benzyl)sulfonyl)-2,2-dimethylpiperidin-4-yl)amino)phenyl)thiophene-2-carboxylic acid (62)

3-(carboxymethoxy)-4-chloro-5-(3-(((4S)-1-((3-(4-((2-(2,6-dioxopiperidin-3-yl)-1-oxoisoindolin-4-yl)glycyl) piperazine-1-carboxamido)benzyl)sulfonyl)-2,2-dimethylpiperidin-4-yl)amino)phenyl)thiophene-2-carboxylic acid (62, 25 mg, 21.02 μmol, 6% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-(1-oxo-4-((2-oxo-2-(piperazin-1-yl)ethyl)amino)isoindolin-2-yl)piperidine-2,6-dione (B-131) in a similar fashion to Example 23, except using excess DIPEA as base and DMF:DCM as the solvent. The residue was purified by reverse phase prep HPLC [Purification method: Column: SunFire C18 (19×150 mm) 5 μm, mobile phase: 0.1% TFA in water and MeCN]. LCMS (ES+): m/z 1019.4 [M+H]⁺. ¹H NMR (400 MHz, DMSO-d₆) δ 11.01 (s, 1H), 8.71 (s, 1H), 7.55-7.52 (m, 1H), 7.47-7.43 (m, 1H), 7.33-7.16 (m, 3H), 7.02-6.97 (m, 2H), 6.87-6.84 (m, 1H), 6.82-6.76 (m, 2H), 6.71-6.66 (m, 1H), 5.11 (dd, J=13.2, 5.2 Hz, 1H), 4.91 (s, 2H), 4.39-4.28 (m, 2H), 4.25 (d, J=13.7 Hz, 1H), 4.18 (d, J=17.0 Hz, 1H), 4.09 (s, 2H), 3.16-3.06 (m, 2H), 2.98-2.85 (m, 3H), 2.65-2.55 (m, 3H), 2.38 (dd, J=13.3, 4.7 Hz, 1H), 2.06-1.97 (m, 1H), 1.91-1.83 (m, 1H), 1.82-1.75 (m, 1H), 1.48 (s, 3H), 1.42-1.35 (m, 5H), 1.15-1.03 (m, 1H).

### Example 63

### 3-(carboxymethoxy)-4-chloro-5-(3-(((4S)-1-((3-(1-(2-((2-(2,6-dioxopiperidin-3-yl)-1-oxoisoindolin-4-yl)oxy) acetyl)piperidine-4-carboxamido)benzyl)sulfonyl)-2,2-dimethylpiperidin-4-yl)amino)phenyl)thiophene-2-carboxylic acid (63)

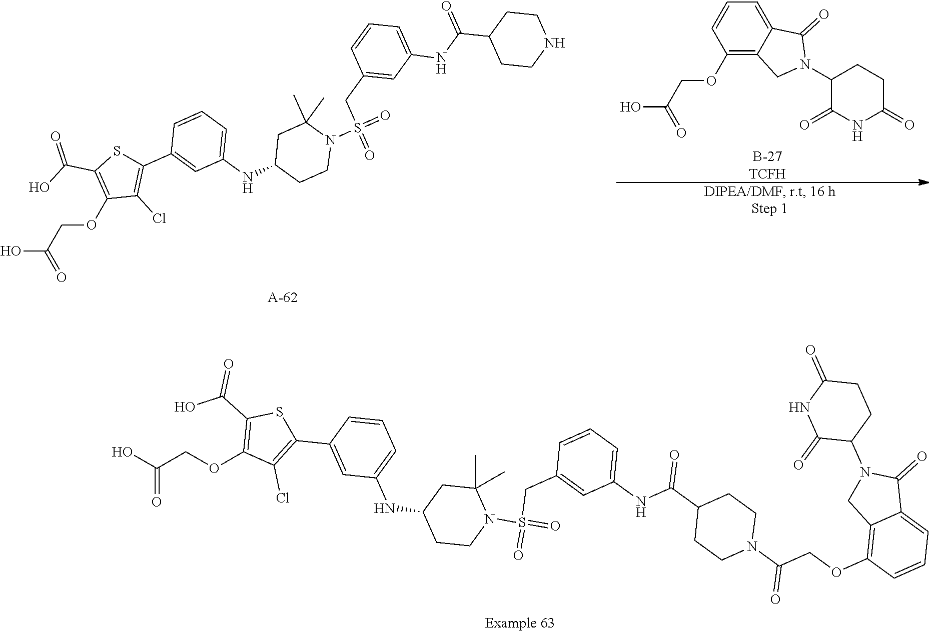 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCNCC2)c1 ; O=C(O)COc1cccc2c1CN(C1CCC(=O)NC1=O)C2=O ; "B-27TCFH" ; "DIPEA/DMF, r.t, 16 hStep 1" ; "A-62" ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)COc3cccc4c3CN(C3CCC(=O)NC3=O)C4=O)CC2)c1]

### Step 1: 3-(carboxymethoxy)-4-chloro-5-(3-(((4S)-1-((3-(1-(2-((2-(2,6-dioxopiperidin-3-yl)-1-oxoisoindolin-4-yl)oxy) acetyl)piperidine-4-carboxamido)benzyl)sulfonyl)-2,2-dimethylpiperidin-4-yl)amino)phenyl)thiophene-2-carboxylic acid (63)

3-(carboxymethoxy)-4-chloro-5-(3-(((4S)-1-((3-(1-(2-((2-(2,6-dioxopiperidin-3-yl)-1-oxoisoindolin-4-yl)oxy) acetyl)piperidine-4-carboxamido)benzyl)sulfonyl)-2,2-dimethylpiperidin-4-yl)amino)phenyl)thiophene-2-carboxylic acid (63, 12 mg, 0.010 mmol, 11% yield) was synthesized from 2-[[2-(2,6-dioxo-3-piperidyl)-1-oxo-isoindolin-4-yl]oxyacetic acid (B-27) and 3-(carboxymethoxy)-4-chloro-5-[3-[[(4S)-2,2-dimethyl-1-[[3-(piperidine-4-carbonylamino)phenyl]methylsulfonyl]-4-piperidyl]amino]phenyl]thiophene-2-carboxylic acid (A-62) in a similar fashion to Example 58. The residue which was purified by reverse phase prep HPLC [Purification method: Column: SunFire C18; 19×150 mm, 5 μm; Mobile phase A: 0.1% TFA in MQ-water; Mobile phase B: Acetonitrile]. LCMS (ES+): m/z 1019.4 [M+H]⁺. ¹H NMR (400 MHz, DMSO-d6): δ 10.99 (s, 1H), 10.03 (s, 1H), 7.69 (s, 1H), 7.61 (d, J=7.60 Hz, 1H), 7.46 (t, J=7.60 Hz, 1H), 7.35-7.25 (m, 2H), 7.23-7.12 (m, 2H), 7.09-7.08 (m, 2H), 6.89 (s, 1H), 6.82 (d, J=Hz, 1H), 6.71 (d, J=Hz, 1H), 5.12 (dd, J=4.80, 13.20 Hz, 2H), 5.10-4.98 (m, 2H), 4.92 (s, 1H), 4.51-4.17 (m, 7H), 3.89 (d, J=14.40 Hz, 2H), 3.53-3.42 (m, 3H), 3.15 (m, 3H), 3.10-2.89 (m, 1H), 2.65-2.60 (m, 2H), 2.02-1.99 (m, 1H), 1.95-1.74 (m, 4H), 1.47 (s, 3H), 1.44 (s, 3H), 1.20-1.05 (m, 1H).

### Example 64

### 3-(carboxymethoxy)-4-chloro-5-(3-(((4S)-1-((3-(4-(3-(2-(2,6-dioxopiperidin-3-yl)-1-oxoisoindolin-4-yl)propanoyl) piperazine-1-carboxamido)benzyl)sulfonyl)-2,2-dimethylpiperidin-4-yl))amino)phenyl)thiophene-2-carboxylic acid (64)

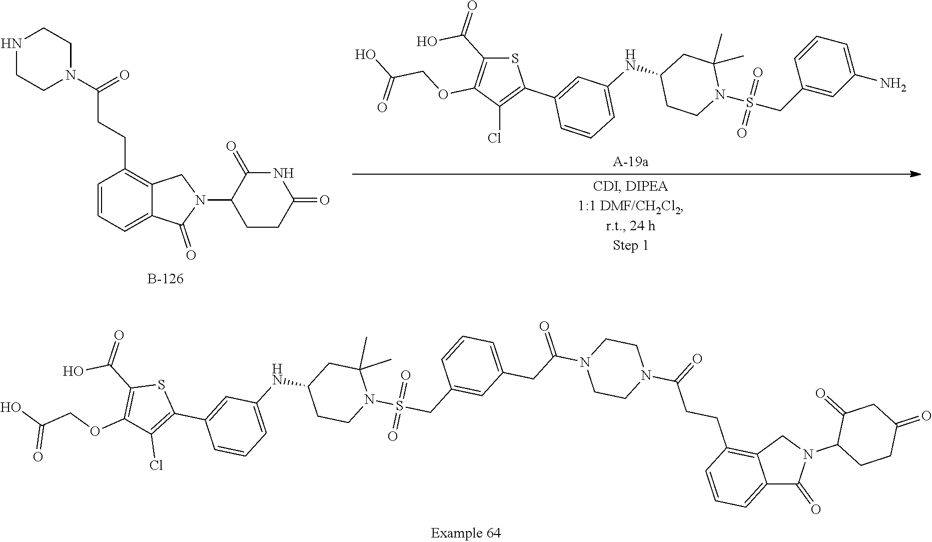 [SMILES: O=C1CCC(N2Cc3c(CCC(=O)N4CCNCC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(CC(=O)N2CCN(C(=O)CCc3cccc4c3CN(C3CCC(=O)CC3=O)C4=O)CC2)c1]

### Step 1: 3-(carboxymethoxy)-4-chloro-5-(3-(((4S)-1-((3-(4-(3-(2-(2,6-dioxopiperidin-3-yl)-1-oxoisoindolin-4-yl)propanoyl) piperazine-1-carboxamido)benzyl)sulfonyl)-2,2-dimethylpiperidin-4-yl))amino)phenyl)thiophene-2-carboxylic acid (64)

3-(carboxymethoxy)-4-chloro-5-(3-(((4S)-1-((3-(4-(3-(2-(2,6-dioxopiperidin-3-yl)-1-oxoisoindolin-4-yl)propanoyl) piperazine-1-carboxamido)benzyl)sulfonyl)-2,2-dimethylpiperidin-4-yl)amino)phenyl)thiophene-2-carboxylic acid (64, 15 mg, 12.31 μmol, 9% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-(1-oxo-4-(3-oxo-3-(piperazin-1-yl)propyl)isoindolin-2-yl)piperidine-2,6-dione (B-126) in a similar fashion to Example 23, except using excess DIPEA as base and DMF:DCM as the solvent. The residue was purified by reverse phase prep HPLC [Purification method: Column: SunFire C18 (19×150 mm) 5 μm, mobile phase: 0.1% TFA in water and MeCN]. LCMS (ES+): m/z 1018.4 [M+H]⁺. ¹H NMR (400 MHz, DMSO-d₆) δ 10.99 (s, 1H), 8.66 (s, 1H), 7.57 (d, J=7.3 Hz, 1H), 7.54-7.50 (m, 2H), 7.49-7.40 (m, 2H), 7.28-7.16 (m, 2H), 6.99 (d, J=7.6 Hz, 1H), 6.86 (s, 1H), 6.80 (d, J=7.5 Hz, 1H), 6.71-6.65 (m, 1H), 5.12 (dd, J=13.3, 5.2 Hz, 1H), 4.91 (s, 2H), 4.52 (d, J=17.2 Hz, 1H), 4.41-4.31 (m, 2H), 4.24 (d, J=13.8 Hz, 1H), 3.16-3.06 (m, 2H), 2.95-2.87 (m, 4H), 2.78-2.71 (m, 3H), 2.64-2.56 (m, 2H), 2.05-1.96 (m, 1H), 1.85 (d, J=12.0 Hz, 1H), 1.77 (d, J=13.0 Hz, 1H), 1.47 (s, 3H), 1.44-1.35 (m, 5H), 1.15-1.00 (m, 1H).

### Example 65

### 3-(carboxymethoxy)-4-chloro-5-[3-[[1-[[3-[[4-[[2-(2,6-dioxo-3-piperidyl)-1-oxo-isoindolin-4-yl]oxypiperidine-1-carbonyl]amino]phenyl]methylsulfonyl]-4-piperidyl]amino]phenyl]thiophene-2-carboxylic acid (65)

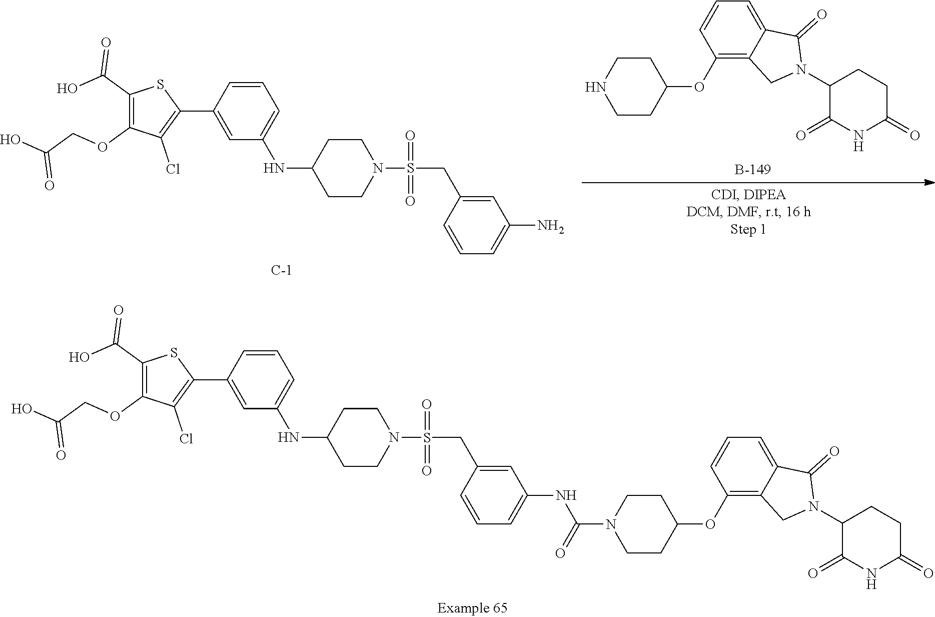

### Step 1: 3-(carboxymethoxy)-4-chloro-5-[3-[[1-[[3-[[4-[[2-(2,6-dioxo-3-piperidyl)-1-oxo-isoindolin-4-yl]oxypiperidine-1-carbonyl]amino]phenyl]methylsulfonyl]-4-piperidyl]amino]phenyl]thiophene-2-carboxylic acid (65)

3-(carboxymethoxy)-4-chloro-5-[3-[[1-[[3-[[4-[[2-(2,6-dioxo-3-piperidyl)-1-oxo-isoindolin-4-yl]oxypiperidine-1-carbonyl]amino]phenyl]methylsulfonyl]-4-piperidyl]amino]phenyl]thiophene-2-carboxylic acid (65, 27 mg, 24.86 μmol, 5% yield) was synthesized from 5-[3-[[1-[(3-aminophenyl)methylsulfonyl]-4-piperidyl]amino]phenyl]-3-(carboxymethoxy)-4-chloro-thiophene-2-carboxylic acid (C-1) and 3-[1-oxo-4-(4-piperidyloxy)isoindolin-2-yl]piperidine-2,6-dione (B-149) in a similar fashion to Example 23, except using excess DIPEA as base and DMF:DCM as the solvent. The residue was purified by reverse phase prep HPLC [Purification method: Column: XSelect, C18; 150×19 mm, 5 μm; Mobile phase A: 0.1% TFA in water and mobile phase B: acetonitrile]. LCMS (ES−): m/z 947.6 [M−H]⁺. ¹H NMR (400 MHz, DMSO-d₆) δ 10.97 (s, 1H), 8.67 (s, 1H), 7.57-7.53 (m, 1H), 7.48 (t, J=7.8 Hz, 1H), 7.43-7.39 (m, 1H), 7.33 (t, J=8.2 Hz, 2H), 7.27-7.17 (m, 2H), 6.99 (d, J=7.6 Hz, 1H), 6.89-6.86 (m, 1H), 6.82 (d, J=7.6 Hz, 1H), 6.74-6.68 (m, 1H), 5.10 (dd, J=13.3, 5.1 Hz, 1H), 4.91 (s, 2H), 4.83-4.74 (m, 1H), 4.39 (d, J=17.5 Hz, 1H), 4.33 (s, 2H), 4.24 (d, J=17.5 Hz, 1H), 3.75 (dd, J=12.3, 6.4 Hz, 3H), 3.02-2.84 (m, 4H), 2.62-2.54 (m, 2H), 2.47-2.39 (m, 1H), 2.02-1.89 (m, 5H), 1.72-1.58 (m, 2H), 1.44-1.29 (m, 2H).

### Example 66

### 3-(carboxymethoxy)-4-chloro-5-(3-(((4R)-1-((3-(1-((2-(2,6-dioxopiperidin-3-yl)-1-oxoisoindolin-4-yl)glycyl)-N-methylpiperidine-4-carboxamido)benzyl)sulfonyl)-2,2-dimethylpiperidin-4-yl)amino)phenyl)thiophene-2-carboxylic acid (66)

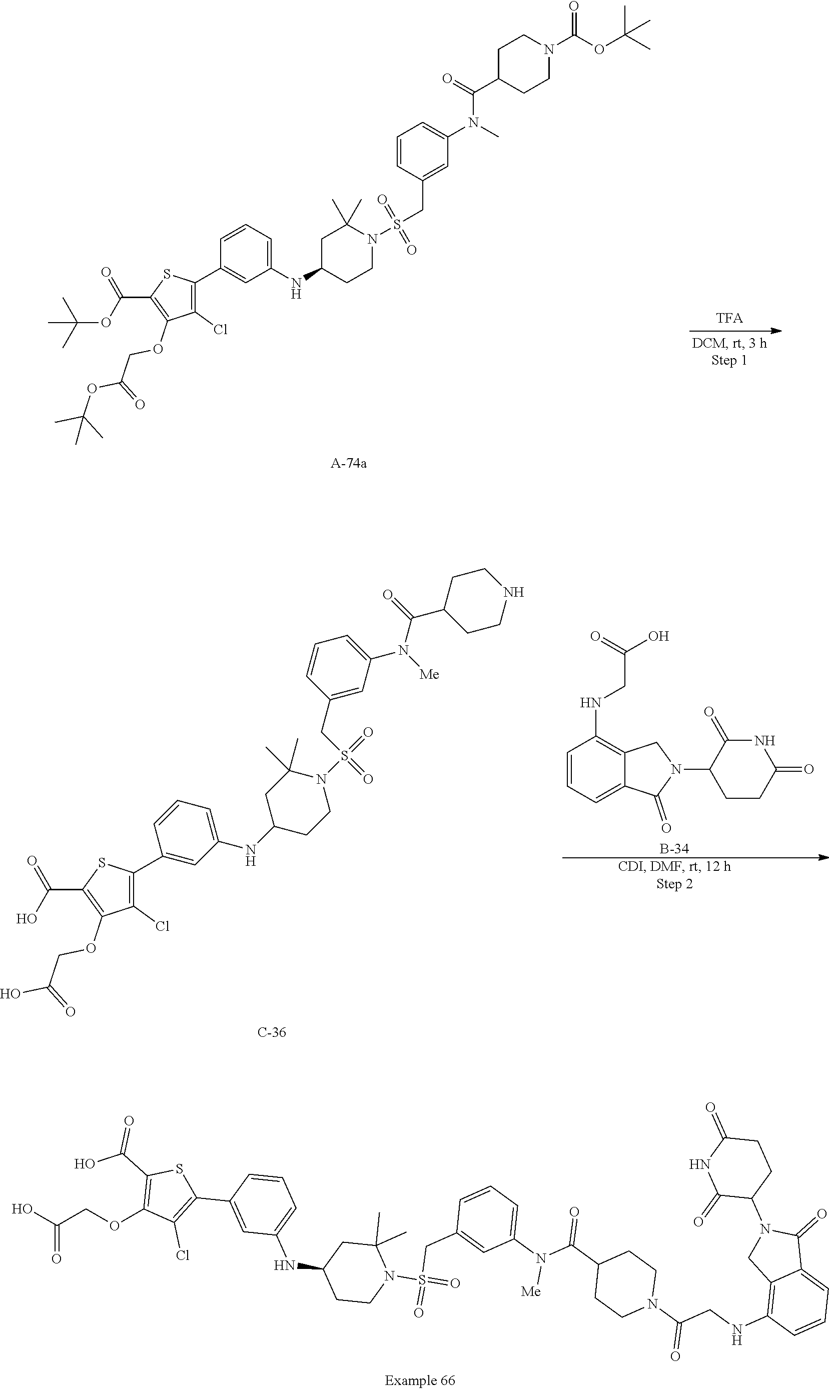

### Step 1: 3-(carboxymethoxy)-4-chloro-5-[3-[(4R)-2,2-dimethyl-1-[[3-[methyl(piperidine-4-carbonyl)amino]phenyl]methylsulfonyl]-4-piperidyl]amino]phenyl]thiophene-2-carboxylic acid (C-36)

3-(carboxymethoxy)-4-chloro-5-[3-[[(4R)-2,2-dimethyl-1-[[3-[methyl(piperidine-4-carbonyl)amino]phenyl]methylsulfonyl]-4-piperidyl]amino]phenyl]thiophene-2-carboxylic acid (C-36, 100 mg, 111.95 μmol, 75.62% yield, TFA salt) was synthesized from tert-butyl (R)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxylate (A-74a) in a similar fashion to Compound C-1, except using 43 eq. of TFA. LCMS (ES+); m/z 733.2 [M+H]⁺.

### Step 2: 3-(carboxymethoxy)-4-chloro-5-(3-(((4R)-1-((3-(1-((2-(2,6-dioxopiperidin-3-yl)-1-oxoisoindolin-4-yl)glycyl)-N-methylpiperidine-4-carboxamido)benzyl)sulfonyl)-2,2-dimethylpiperidin-4-yl)amino)phenyl)thiophene-2-carboxylic acid (66)

3-(carboxymethoxy)-4-chloro-5-(3-(((4R)-1-((3-(1-((2-(2,6-dioxopiperidin-3-yl)-1-oxoisoindolin-4-yl)glycyl)-N-methylpiperidine-4-carboxamido)benzyl)sulfonyl)-2,2-dimethylpiperidin-4-yl)amino)phenyl)thiophene-2-carboxylic acid (66, 23 mg, 19.77 μmol, 16% yield, TFA salt) was synthesized from 2-[[2-(2,6-dioxo-3-piperidyl)-1-oxo-isoindolin-4-yl]amino]acetic acid (B-34) and 3-(carboxymethoxy)-4-chloro-5-[3-[(4R)-2,2-dimethyl-1-[[3-[methyl(piperidine-4-carbonyl)amino]phenyl]methylsulfonyl]-4-piperidyl]amino]phenyl]thiophene-2-carboxylic acid (C-36) in a similar fashion to Example 23, except using DMF as the solvent. The residue was purified by reverse phase prep HPLC [Purification method: Column: SunFire C18 (19×150 mm) 5 μm, mobile phase: 0.1% TFA in water and MeCN]. LCMS (ES+): m/z 1032.4 [M+H]⁺. ¹H NMR (400 MHz, DMSO-d₆) δ 10.99 (s, 1H), 7.55-7.41 (m, 2H), 7.41-7.35 (m, 2H), 7.26 (t, J=7.7 Hz, 1H), 7.20 (t, J=7.9 Hz, 1H), 6.97 (d, J=7.4 Hz, 1H), 6.89 (s, 1H), 6.82 (d, J=7.6 Hz, 1H), 6.75-6.68 (m, 2H), 5.10 (dd, J=13.2, 5.1 Hz, 1H), 4.91 (s, 2H), 4.53 (d, J=13.7 Hz, 1H), 4.38 (d, J=13.6 Hz, 1H), 4.33-4.24 (m, 2H), 4.16 (d, J=17.0 Hz, 1H), 4.06-3.92 (m, 3H), 3.20-3.09 (m, 4H), 2.98-2.84 (m, 2H), 2.66-2.56 (m, 2H), 2.40-2.23 (m, 2H), 2.05-1.98 (m, 1H), 1.95-1.85 (m, 1H), 1.80 (d, 1H), 1.61 (s, 3H), 1.42 (s, 3H), 1.38 (s, 3H), 1.11 (s, 1H).

### Example 67

### 3-(carboxymethoxy)-4-chloro-5-(3-(((4S)-1-((3-(1-((2-(2,6-dioxopiperidin-3-yl)-1-oxoisoindolin-4-yl)glycyl)-N-methylpiperidine-4-carboxamido)benzyl)sulfonyl)-2,2-dimethylpiperidin-4-yl)amino)phenyl)thiophene-2-carboxylic acid (67)

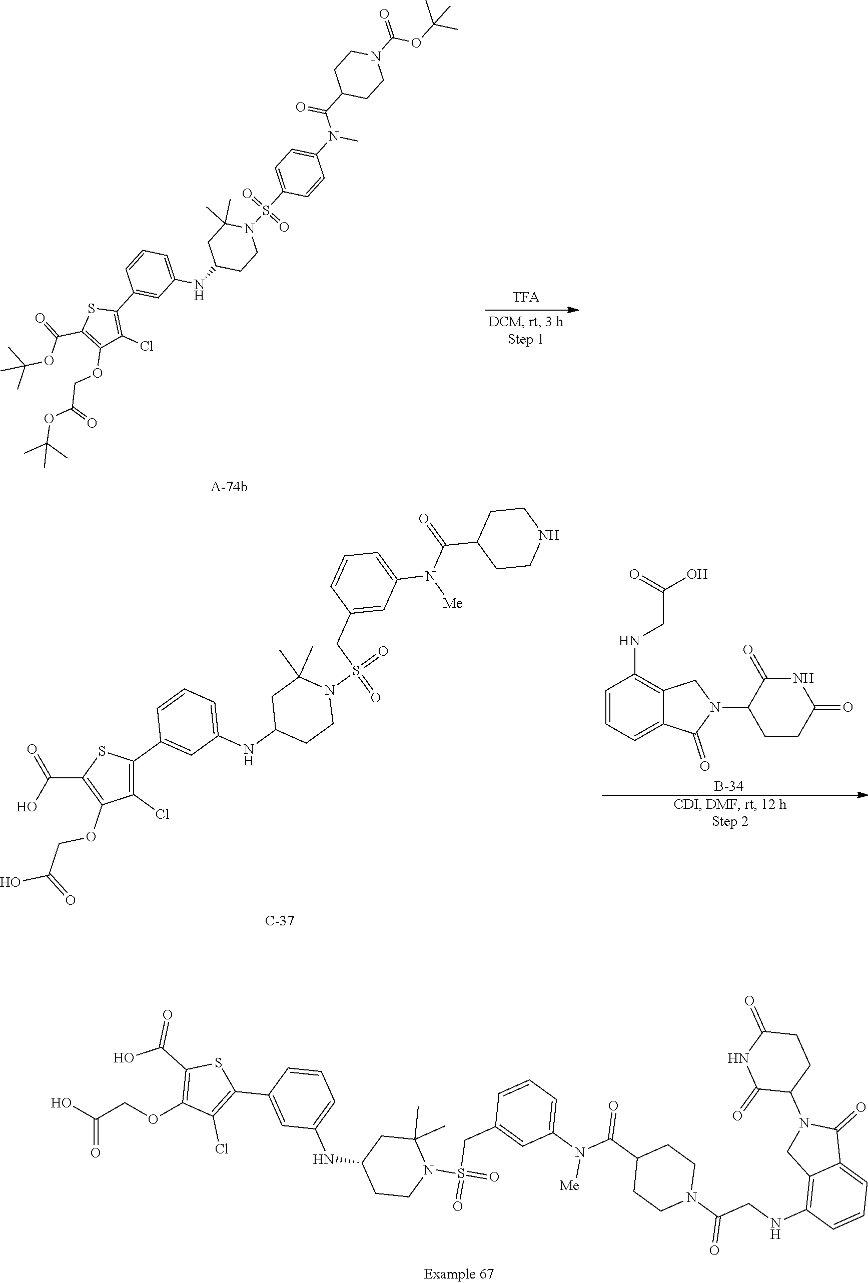

### 3-(carboxymethoxy)-4-chloro-5-(3-(((4S)-1-((3-(1-((2-(2,6-dioxopiperidin-3-yl)-1-oxoisoindolin-4-yl)glycyl)-N-methylpiperidine-4-carboxamido)benzyl)sulfonyl)-2,2-dimethylpiperidin-4-yl)amino)phenyl)thiophene-2-carboxylic acid (67)

3-(carboxymethoxy)-4-chloro-5-(3-(((4S)-1-((3-(1-((2-(2,6-dioxopiperidin-3-yl)-1-oxoisoindolin-4-yl)glycyl)-N-methylpiperidine-4-carboxamido)benzyl)sulfonyl)-2,2-dimethylpiperidin-4-yl)amino)phenyl)thiophene-2-carboxylic acid (67, 10.0 mg, 8.59 μmol, 9% yield, TFA salt) was synthesized from tert-butyl(S)-4-((3-(((4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)phenyl)(methyl)carbamoyl)piperidine-1-carboxy late (A-74b) following the same two step procedure used to synthesize Example 66. LCMS (ES+): m/z 1032.4 [M+H]⁺. ¹H NMR (400 MHz, DMSO-d₆) δ 10.99 (s, 1H), 7.55-7.48 (m, 1H), 7.43 (d, J=7.4 Hz, 1H), 7.41-7.36 (m, 2H), 7.26 (t, J=7.7 Hz, 1H), 7.20 (t, J=7.9 Hz, 1H), 6.97 (d, J=7.4 Hz, 1H), 6.89-6.86 (m, 1H), 6.83-6.78 (m, 1H), 6.75-6.67 (m, 2H), 5.10 (dd, J=13.2, 5.1 Hz, 1H), 4.91 (s, 2H), 4.54 (d, J=13.6 Hz, 1H), 4.38 (d, J=13.6 Hz, 1H), 4.34-4.24 (m, 2H), 4.16 (d, J=17.1 Hz, 1H), 4.07-3.79 (m, 5H), 3.19-3.12 (m, 4H), 2.98-2.85 (m, 2H), 2.77-2.68 (m, 1H), 2.64-2.56 (m, 1H), 2.40-2.22 (m, 3H), 2.05-1.96 (m, 1H), 1.94-1.86 (m, 1H), 1.80 (d, J=12.6 Hz, 1H), 1.61 (s, 3H), 1.42 (s, 3H), 1.39 (s, 3H), 1.35-1.29 (m, 1H), 1.17-1.04 (m, 1H).

### Example 68

### 3-(carboxymethoxy)-4-chloro-5-[3-[[(4S)-1-[[3-[[4-[3-(2,6-dioxo-3-piperidyl)-2-oxo-1,3-benzoxazol-6-yl]piperidine-1-carbonyl]amino]phenyl]methylsulfonyl]-2,2-dimethyl-4-piperidyl]amino]phenyl]thiophene-2-carboxylic acid (68)

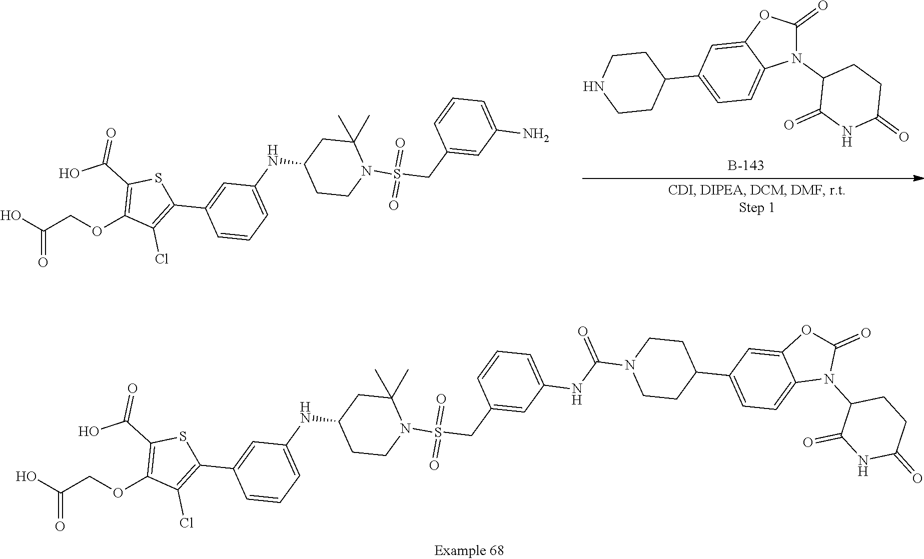 [SMILES: CC1(C)C[C@@H](Nc2cccc(-c3sc(C(=O)O)c(OCC(=O)O)c3Cl)c2)CCN1S(=O)(=O)Cc1cccc(N)c1 ; O=C1CCC(n2c(=O)oc3cc(C4CCNCC4)ccc32)C(=O)N1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)oc(=O)n4C3CCC(=O)NC3=O)CC2)c1]

### Step 1: 3-(carboxymethoxy)-4-chloro-5-[3-[[(4S)-1-[[3-[[4-[3-(2,6-dioxo-3-piperidyl)-2-oxo-1,3-benzoxazol-6-yl]piperidine-1-carbonyl]amino]phenyl]methylsulfonyl]-2,2-dimethyl-4-piperidyl]amino]phenyl]thiophene-2-carboxylic acid (68)

3-(carboxymethoxy)-4-chloro-5-[3-[[(4S)-1-[3-[4-[3-(2,6-dioxo-3-piperidyl)-2-oxo-1,3-benzoxazol-6-yl]piperidine-1-carbonyl]amino]phenyl]methylsulfonyl]-2,2-dimethyl-4-piperidyl]amino]phenyl]thiophene-2-carboxylic acid (68, 17 mg, 15.60 μmol, 8% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[2-oxo-6-(4-piperidyl)-1,3-benzoxazol-3-yl]piperidine-2,6-dione (B-143) in a similar fashion to Example 23, except using excess DIPEA as base and DMF:DCM as the solvent. The residue was purified by reverse phase column chromatography using the method: Column: SunFire C18 (190×150 mm) 5 μm; Mobile phase A: 0.1% TFA in MQ-water; Mobile phase B: Acetonitrile. LCMS (ESI): m/z 963.4 [M+H]⁺. ¹H NMR (400 MHz, DMSO-d₆) δ 11.21 (s, 1H), 8.62 (s, 1H), 7.56 (s, 1H), 7.48-7.42 (m, 1H), 7.33 (s, 1H), 7.28-7.15 (m, 3H), 7.11 (d, J=8.2 Hz, 1H), 6.98 (d, J=7.6 Hz, 1H), 6.89-6.85 (m, 1H), 6.81 (d, J=7.6 Hz, 1H), 6.71-6.67 (m, 1H), 5.34 (dd, J=12.9, 5.3 Hz, 1H), 4.90 (s, 2H), 4.40-4.21 (m, 4H), 3.18-3.06 (m, 2H), 2.97-2.82 (m, 3H), 2.82-2.72 (m, 2H), 2.71-2.61 (m, 3H), 2.20-2.10 (m, 1H), 1.89-1.72 (m, 4H), 1.67-1.53 (m, 2H), 1.48 (s, 3H), 1.45-1.37 (m, 4H), 1.18-1.02 (m, 1H).

### Example 69

### 3-(carboxymethoxy)-4-chloro-5-[3-[[(4S)-1-[[3-[2-[4-[1-(2,6-dioxo-3-piperidyl)-3-methyl-2-oxo-benzimidazol-S-yl]-1-piperidyl]acetyl]amino]phenyl]methylsulfonyl]-2,2-dimethyl-4-piperidyl]amino]phenyl]thiophene-2-carboxylic acid (69)

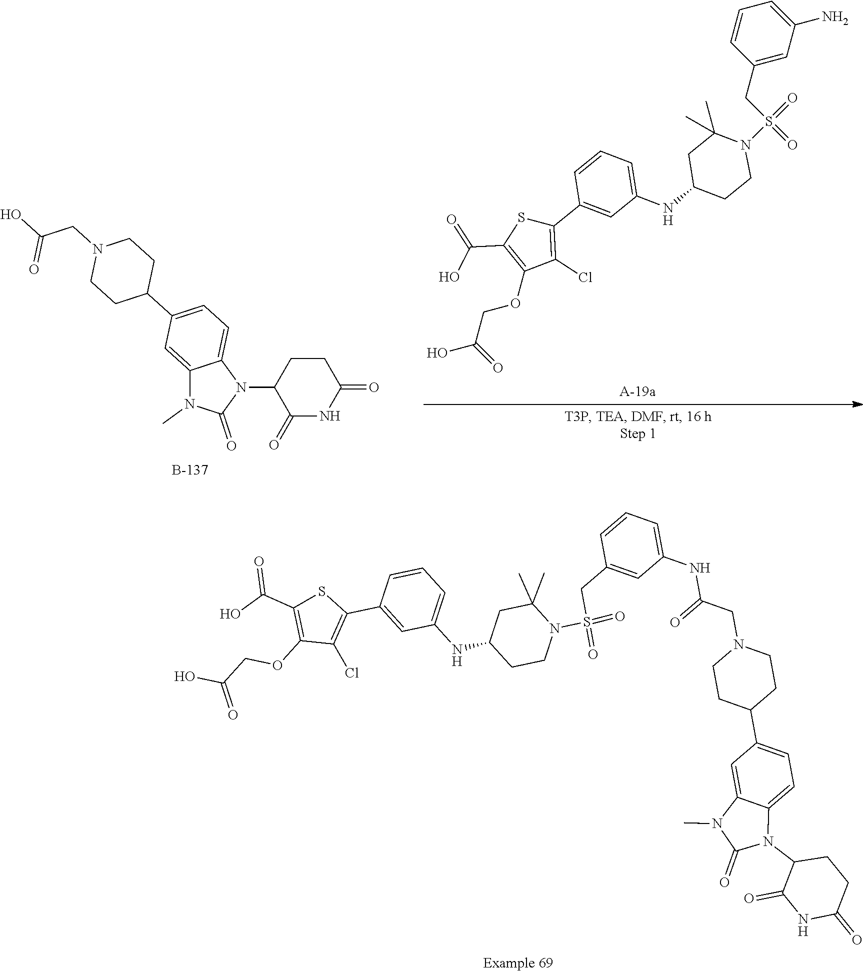 [SMILES: Cn1c(=O)n(C2CCC(=O)NC2=O)c2ccc(C3CCN(CC(=O)O)CC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(C3CCN(CC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[2-[4-[1-(2,6-dioxo-3-piperidyl)-3-methyl-2-oxo-benzimidazol-5-yl]-1-piperidyl]acetyl]amino]phenyl]methylsulfonyl]-2,2-dimethyl-4-piperidyl]amino]phenyl]thiophene-2-carboxylic acid (69)

3-(carboxymethoxy)-4-chloro-5-[3-[[(4S)-1-[[3-[2-[4-[1-(2,6-dioxo-3-piperidyl)-3-methyl-2-oxo-benzimidazol-5-yl]-1-piperidyl]acetyl]amino]phenyl]methylsulfonyl]-2,2-dimethyl-4-piperidyl]amino]phenyl]thiophene-2-carboxylic acid (69, 9.8 mg, 8.52 μmol, 9% yield, TFA salt) was synthesized from 2-[4-[1-(2,6-dioxo-3-piperidyl)-3-methyl-2-oxo-benzimidazol-5-yl]-1-piperidyl]acetic acid (B-137) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Compound C-6. The residue was purified by reverse phase Prep HPLC (Column: SunFire C18, 19×150 mm, 5 μm; Mobile phase A-0.1 TFA in water; Mobile phase B-MeCN). LCMS (ES+): m/z 990.4 [M+H]⁺.

¹H NMR (400 MHz, DMSO-d₆) δ 11.10 (s, 1H), 10.55 (s, 1H), 7.71 (s, 1H), 7.63 (d, J=8.3 Hz, 1H), 7.39 (t, J=7.9 Hz, 1H), 7.23-7.14 (m, 2H), 7.11-7.03 (m, 2H), 6.92 (d, J=8.3 Hz, 1H), 6.85 (s, 1H), 6.79 (d, J=7.5 Hz, 1H), 6.67 (d, J=8.3 Hz, 1H), 5.75-5.68 (m, 1H), 5.35 (dd, J=12.7, 5.4 Hz, 1H), 4.83 (S, 2H), 4.46 (d, J=13.7 Hz, 1H), 4.34 (d, J=13.7 Hz, 1H), 4.08 (s, 1H), 3.12 (t, J=12.4 Hz, 3H), 2.88 (q, J=17.9, 15.5 Hz, 5H), 2.77-2.63 (m, 2H), 2.12-1.73 (m, 8H), 1.47 (s, 3H), 1.39 (s, 3H), 1.16-1.01 (m, 1H).

### Example 70

### 3-(Carboxymethoxy)-4-chloro-5-[3-[[(4S)-1-[[3-[[2-[4-[3-(2,6-dioxo-3-piperidyl)-2-oxo-1,3-benzoxazol-6-yl]-1-piperidyl]acetyl]amino]phenyl]methylsulfonyl]-2,2-dimethyl-4-piperidyl]amino]phenyl]thiophene-2-carboxylic acid (70)

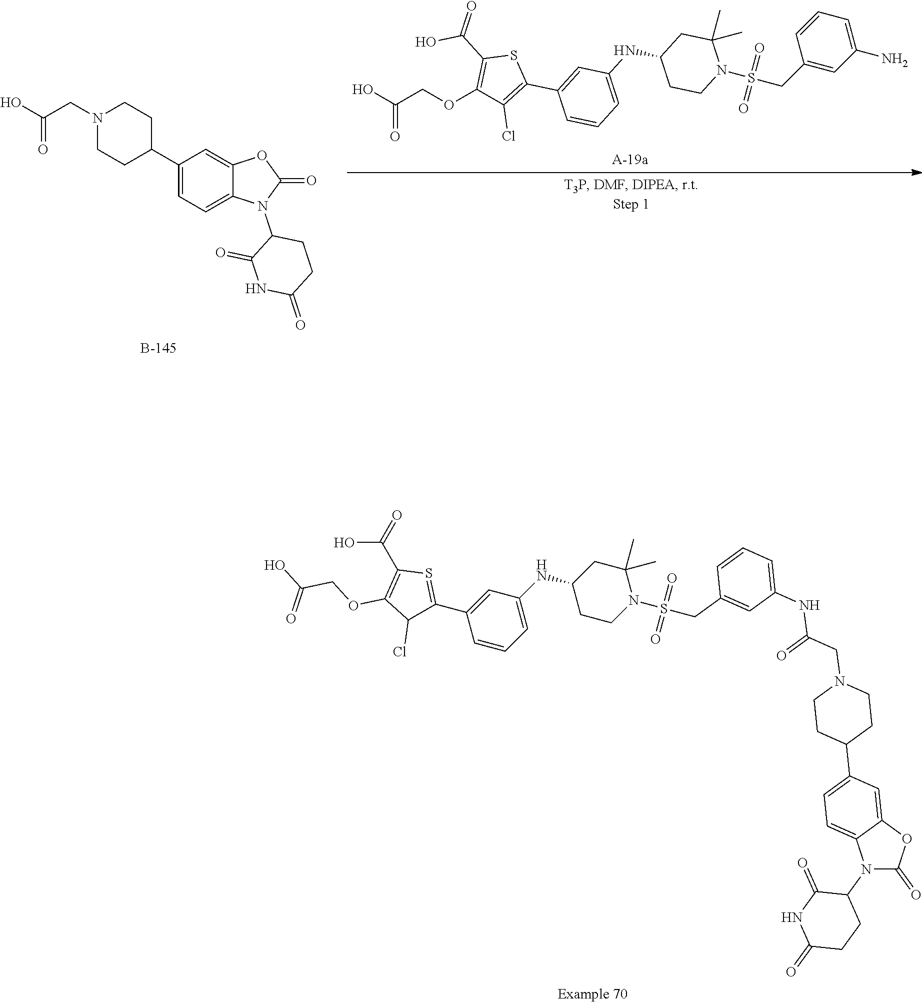

### Step 1: 3-(carboxymethoxy)-4-chloro-5-[3-[[(4S)-1-[[3-[[2-[4-[3-(2,6-dioxo-3-piperidyl)-2-oxo-1,3-benzoxazol-6-yl]-1-piperidyl]acetyl]amino]phenyl]methylsulfonyl]-2,2-dimethyl-4-piperidyl]amino]phenyl]thiophene-2-carboxylic acid (70)

3-(carboxymethoxy)-4-chloro-5-[3-[[(4S)-1-[[3-[[2-[4-[3-(2,6-dioxo-3-piperidyl)-2-oxo-1,3-benzoxazol-6-yl]-1-piperidyl]acetyl]amino]phenyl]methylsulfonyl]-2,2-dimethyl-4-piperidyl]amino]phenyl]thiophene-2-carboxylic acid (70, 35 mg, 0.031 mmol, 8% yield) was synthesized from 2-[4-[3-(2,6-dioxo-3-piperidyl)-2-oxo-1,3-benzoxazol-6-yl]-1-piperidyl]acetic acid (B-145) and 5-[3-[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Compound C-6, except using DIPEA as the base. The residue was purified by reverse phase prep HPLC SunFire C18 (19×150 mm) 5 μm with Solvent A: 0.1% TFA in water; Solvent B: Acetonitrile. LCMS (ESI): m/z 976.6 [M+H]⁺. ¹H NMR (400 MHz, DMSO-d₆) δ 11.23 (s, 1H), 10.68 (s, 1H), 9.78 (s, 1H), 7.69 (s, 1H), 7.63 (d, J=8.1 Hz, 1H), 7.40 (t, J=7.9 Hz, 1H), 7.29 (s, 1H), 7.26-7.21 (m, 1H), 7.18 (d, J=8.0 Hz, 2H), 7.09 (d, J=8.1 Hz, 1H), 6.87 (d, J=2.1 Hz, 1H), 6.80 (d, J=7.6 Hz, 1H), 6.72-6.66 (m, 1H), 5.37 (dd, J=13.1, 5.3 Hz, 1H), 4.91 (s, 2H), 4.46 (d, J=13.8 Hz, 1H), 4.34 (d, J=13.8 Hz, 1H), 4.19 (s, 2H), 3.66-3.59 (m, 2H), 3.57-3.38 (m, 3H), 3.32-3.19 (m, 2H), 3.12 (t, J=12.5 Hz, 1H), 2.96-2.84 (m, 2H), 2.72-2.61 (m, 3H), 2.20-2.12 (m, 1H), 2.06-1.94 (m, 4H), 1.88 (d, J=11.6 Hz, 1H), 1.80 (d, J=12.6 Hz, 1H), 1.47 (s, 3H), 1.43-1.34 (m, 4H), 1.15-0.99 (m, 1H).

### Example 71

### 3-(carboxymethoxy)-4-chloro-5-(3-((1-((3-(3-(4-((2-(2,6-dioxopiperidin-3-yl)-1-oxoisoindolin-4-yl)amino)piperidin-1-yl)-N-methylpropanamido)benzyl)sulfonyl)-2,2-dimethylpiperidin-4-yl)amino)phenyl)thiophene-2-carboxylic acid (71)

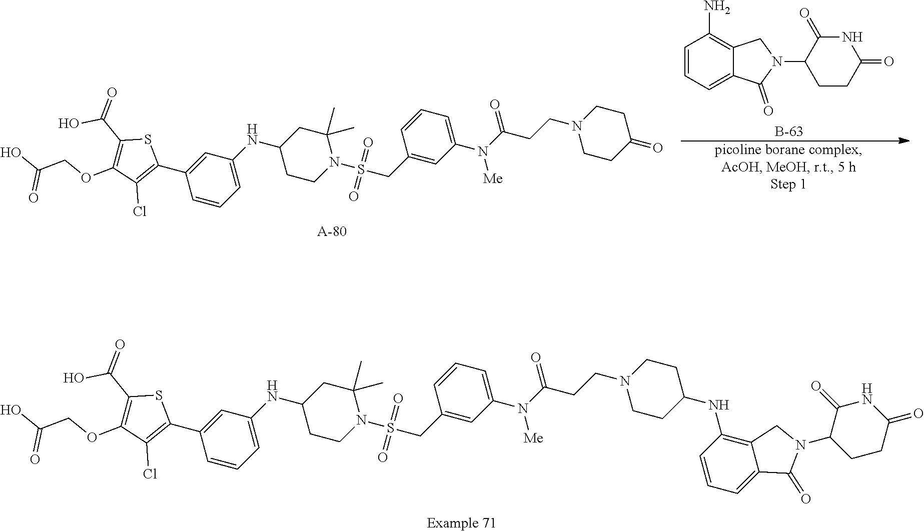

### Step 1: 3-(carboxymethoxy)-4-chloro-5-(3-((1-((3-(3-(4-((2-(2,6-dioxopiperidin-3-yl)-1-oxoisoindolin-4-yl)amino)piperidin-1-yl)-N-methylpropanamido)benzyl)sulfonyl)-2,2-dimethylpiperidin-4-yl)amino)phenyl)thiophene-2-carboxylic acid (71)

3-(carboxymethoxy)-4-chloro-5-(3-((1-((3-(3-(4-((2-(2,6-dioxopiperidin-3-yl)-1-oxoisoindolin-4-yl)amino)piperidin-1-yl)-N-methylpropanamido)benzyl)sulfonyl)-2,2-dimethylpiperidin-4-yl)amino)phenyl)thiophene-2-carboxylic acid (71, 30 mg, 28.15 μmol, 15% yield, Formic acid salt) was synthesized from 3-(carboxymethoxy)-4-chloro-5-[3-[[2,2-dimethyl-1-[[3-[methyl-[3-(4-oxo-1-piperidyl)propanoyl]amino]phenyl]methylsulfonyl]-4-piperidyl]amino]phenyl]thiophene-2-carboxylic acid (A-80) and 3-(4-amino-1-oxo-isoindolin-2-yl)piperidine-2,6-dione (B-63) in a similar fashion to Compound C-29. The residue was purified by reverse phase prep HPLC [Purification method: Column: SunFire C18 (19×150 mm) 5 μm, mobile phase: 0.1% Formic acid in water and MeCN]. LCMS (ES+): m/z 1018.4 [M+H]⁺. ¹H NMR (400 MHz, DMSO-d₆) δ 11.02 (s, 1H), 7.55-7.35 (m, 4H), 7.27 (t, J=7.9 Hz, 1H), 7.16 (t, J=7.9 Hz, 1H), 6.95 (d, J=7.4 Hz, 1H), 6.80 (s, 2H), 6.77 (d, J=7.8 Hz, 1H), 6.64 (d, J=8.3 Hz, 1H), 5.74 (s, 1H), 5.46 (s, 1H), 5.12 (dd, J=13.3, 5.1 Hz, 1H), 4.61 (s, 2H), 4.51 (d, J=13.6 Hz, 1H), 4.38 (d, J=13.7 Hz, 1H), 4.22 (d, J=17.2 Hz, 1H), 4.12 (d, J=17.4 Hz, 1H), 3.56-3.44 (m, 3H), 3.23-3.07 (m, 8H), 3.00-2.86 (m, 2H), 2.65-2.58 (m, 1H), 2.30-2.22 (m, 1H), 2.07-1.88 (m, 4H), 1.78 (d, J=12.9 Hz, 1H), 1.44 (s, 3H), 1.41-1.29 (m, 4H), 1.13-0.98 (m, 1H).

### Example 72

### 3-(carboxymethoxy)-4-chloro-5-[3-[[(4S)-1-[[3-[[4-[[2-(2,6-dioxo-3-piperidyl)-1-oxo-isoindolin-4-yl]oxypiperidine-1-carbonyl]amino]phenyl]methylsulfonyl]-2,2-dimethyl-4-piperidyl]amino]phenyl]thiophene-2-carboxylic acid (72)

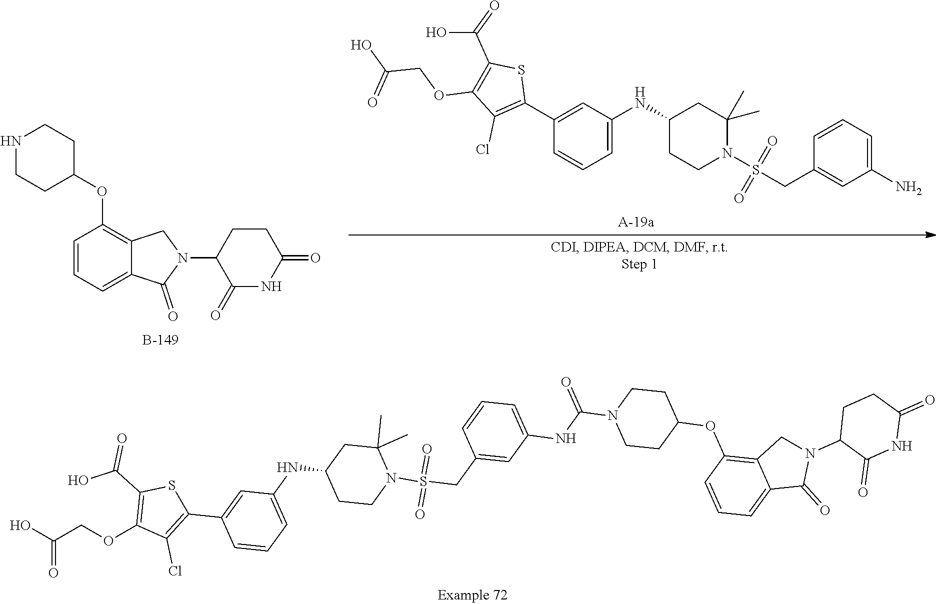 [SMILES: O=C1CCC(N2Cc3c(OC4CCNCC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(Oc3cccc4c3CN(C3CCC(=O)NC3=O)C4=O)CC2)c1]

### Step 1: 3-(carboxymethoxy)-4-chloro-5-[3-[[(4S)-1-[[3-[[4-[[2-(2,6-dioxo-3-piperidyl)-1-oxo-isoindolin-4-yl]oxypiperidine-1-carbonyl]amino]phenyl]methylsulfonyl]-2,2-dimethyl-4-piperidyl]amino]phenyl]thiophene-2-carboxylic acid (72)

3-(carboxymethoxy)-4-chloro-5-[3-[[(4S)-1-[[3-[[4-[[2-(2,6-dioxo-3-piperidyl)-1-oxo-isoindolin-4-yl]oxypiperidine-1-carbonyl]amino]phenyl]methylsulfonyl]-2,2-dimethyl-4-piperidyl]amino]phenyl]thiophene-2-carboxylic acid (72, 4.6 mg, 0.004 mmol, 7% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-(1-oxo-4-(piperidin-4-yloxy)isoindolin-2-yl)piperidine-2,6-dione (B-149) in a similar fashion to Example 23, except using excess DIPEA as base and DMF:DCM as the solvent. The residue was purified by reverse phase Prep-HPLC with the method using a SUNFIRE C18 (19×150 mm), 5 μm; mobile phase A: 0.1% TFA in water and mobile phase B: Acetonitrile, RT=1.2, flow rate=15 mL. LCMS (ESI): m/z 977.2 [M+H]⁺. ¹H NMR (400 MHz, DMSO-d₆) δ 10.97 (s, 1H), 8.66 (s, 1H), 7.53 (s, 1H), 7.48 (t, J=7.8 Hz, 1H), 7.46-7.42 (m, 1H), 7.35-7.30 (m, 2H), 7.27-7.16 (m, 2H), 6.98 (d, J=7.5 Hz, 1H), 6.88-6.85 (m, 1H), 6.81 (d, J=7.5 Hz, 1H), 6.72-6.66 (m, 1H), 5.10 (dd, J=13.2, 5.1 Hz, 1H), 4.91 (s, 2H), 4.79 (dd, J=8.1, 4.2 Hz, 1H), 4.44-4.20 (m, 4H), 3.80-3.71 (m, 3H), 3.16-3.06 (m, 3H), 2.97-2.83 (m, 2H), 2.62-2.54 (m, 1H), 2.02-1.92 (m, 3H), 1.88-1.73 (m, 2H), 1.72-1.59 (m, 2H), 1.47 (s, 3H), 1.45-1.36 (m, 4H), 1.17-1.01 (m, 1H).

### Example 73

### 3-(carboxymethoxy)-4-chloro-5-(3-((1-((3-(1-((2-(2,6-dioxopiperidin-3-yl)-1-oxoisoindolin-4-yl)glycyl)piperidine-4-carboxamido)-2-fluorobenzyl)sulfonyl)piperidin-4-yl)amino)phenyl)thiophene-2-carboxylic acid (73)

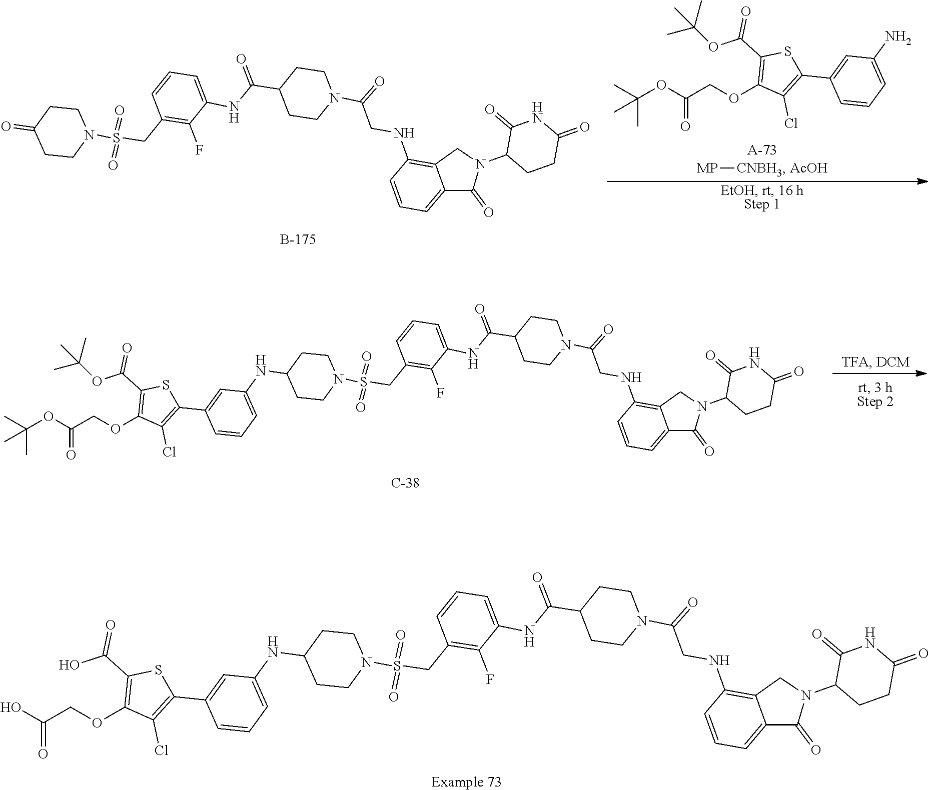

### Step 1: tert-butyl 3-(2-tert-butoxy-2-oxo-ethoxy)-4-chloro-5-[3-[[1-[[3-[[1-[2-[[2-(2,6-dioxo-3-piperidyl)-1-oxo-isoindolin-4-yl]amino]acetyl]piperidine-4-carbonyl]amino]-2-fluoro-phenyl]methylsulfonyl]-4-piperidyl]amino]phenyl]thiophene-2-carboxylate (C-38)

Into a 20 mL pressure tube containing a well-stirred solution of 1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]piperidine-4-carboxamide (B-175, 175 mg, 0.251 mmol) and tert-butyl 5-(3-aminophenyl)-3-(2-tert-butoxy-2-oxo-ethoxy)-4-chloro-thiophene-2-carboxylate (A-73, 100.46 mg, 0.228 mmol) in anhydrous EtOH (5 mL) were added acetic acid (2.74 mg, 0.045 mmol, 2.61 μL) and MP-cyanoborohydride (156.18 mg, 1.37 mmol) at room temperature under nitrogen atmosphere. The resulting mixture was stirred at room temperature for 16 h. The reaction mixture was filtered, and the resin was washed with ethanol (10 mL). The filtrate was evaporated under reduced pressure to afford a crude residue, which was purified by reverse phase prep HPLC [Column: SunFire C18 (190×150) mm, 5 μm; Mobile phase A: 0.1% TFA in MQ-water; Mobile phase B: Acetonitrile] to afford tert-butyl 3-(2-tert-butoxy-2-oxo-ethoxy)-4-chloro-5-[3-[[1-[[3-[[1-[2-[[2-(2,6-dioxo-3-piperidyl)-1-oxo-isoindolin-4-yl]amino]acetyl]piperidine-4-carbonyl]amino]-2-fluoro-phenyl]methylsulfonyl]-4-piperidyl]amino]phenyl]thiophene-2-carboxylate (C-38, 60 mg, 0.047 mmol, 21% yield, TFA salt) as an off-white solid. LCMS (ES+): m/z 1120.1 [M+H]⁺.

### Step 2: 3-(carboxymethoxy)-4-chloro-5-(3-((1-((3-(1-((2-(2,6-dioxopiperidin-3-yl)-1-oxoisoindolin-4-yl)glycyl)piperidine-4-carboxamido)-2-fluorobenzyl)sulfonyl)piperidin-4-yl)amino)phenyl)thiophene-2-carboxylic acid (73)

3-(carboxymethoxy)-4-chloro-5-(3-((1-((3-(1-((2-(2,6-dioxopiperidin-3-yl)-1-oxoisoindolin-4-yl)glycyl)piperidine-4-carboxamido)-2-fluorobenzyl)sulfonyl)piperidin-4-yl)amino)phenyl)thiophene-2-carboxylic acid (73, 30 mg, 0.026 mmol, 54% yield, TFA salt) was synthesized from tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]piperidine-4-carbonyl]amino]-2-fluoro-phenyl]methylsulfonyl]-4-piperidyl]amino]phenyl]thiophene-2-carboxylate (C-38) in a similar fashion to Compound C-1, except using 260 eq. of TFA. The residue was purified by reverse phase prep HPLC [Purification method: Column: SunFire C18 (19×150) mm, 5 μm; Mobile phase A: 0.1% TFA in MQ-water; Mobile phase B: Acetonitrile]. LCMS (ES+): m/z 1008.3 [M+H]⁺. ¹H NMR (400 MHz, DMSO-d₆) δ 11.00 (s, 1H), 9.83 (s, 1H), 7.82 (t, J=7.6 Hz, 1H), 7.29 (t, J=7.7 Hz, 1H), 7.26-7.14 (m, 3H), 6.98 (d, J=7.4 Hz, 1H), 6.90-6.87 (m, 1H), 6.82 (d, J=7.6 Hz, 1H), 6.78 (d, J=8.1 Hz, 1H), 6.75-6.70 (m, 1H), 5.12 (dd, J=13.3, 5.1 Hz, 1H), 4.92 (s, 2H), 4.46 (s, 2H), 4.40 (d, J=12.7 Hz, 1H), 4.31 (d, J=17.0 Hz, 1H), 4.18 (d, J=17.0 Hz, 1H), 4.06 (s, 2H), 3.60-3.51 (m, 2H), 3.50-3.40 (m, 1H), 3.11 (t, J=12.6 Hz, 1H), 3.05-2.86 (m, 4H), 2.84-2.69 (m, 2H), 2.65-2.57 (m, 2H), 2.38 (dd, J=13.1, 4.4 Hz, 1H), 2.06-1.93 (m, 3H), 1.91-1.81 (m, 2H), 1.73-1.59 (m, 1H), 1.55-1.31 (m, 3H).

### Example 74

### 3-(carboxymethoxy)-4-chloro-5-(3-(((4S)-1-((3-(1-(2-((2-(2,6-dioxopiperidin-3-yl)-1-oxoisoindolin-4-yloxy) acetyl) azetidine-3-carboxamido)benzyl)sulfonyl)-2,2-dimethylpiperidin-4-yl)amino)phenyl)thiophene-2-carboxylic acid (74)

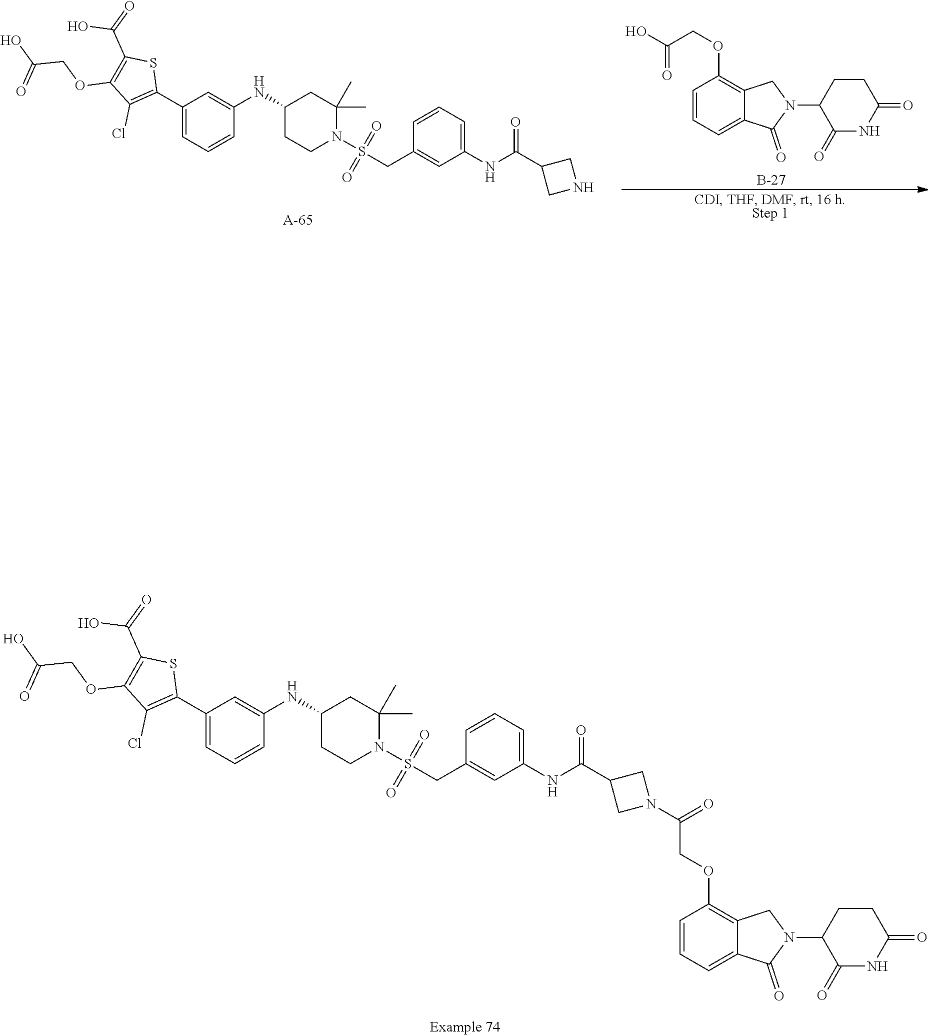

### Step 1: 3. (carboxymethoxy)-4-chloro-5-(3-(((4S)-1-((3-(1-(2-((2-(2,6-dioxopiperidin-3-yl)-1-oxoisoindolin-4-yl)oxy) acetyl) azetidine-3-carboxamido)benzyl)sulfonyl)-2,2-dimethylpiperidin-4-yl)amino)phenyl)thiophene-2-carboxylic acid (74)

3-(carboxymethoxy)-4-chloro-5-(3-(((4S)-1-((3-(1-(2-((2-(2,6-dioxopiperidin-3-yl)-1-oxoisoindolin-4-yl)oxy) acetyl) azetidine-3-carboxamido)benzyl)sulfonyl)-2,2-dimethylpiperidin-4-yl)amino)phenyl)thiophene-2-carboxylic acid (74, 5.26 mg, 4.71 μmol, 15% yield) was synthesized from 2-[[2-(2,6-dioxo-3-piperidyl)-1-oxo-isoindolin-4-yl]oxyacetic acid (B-27) and 5-[3-[[(4S)-1-[[3-(azetidine-3-carbonylamino)phenyl]methylsulfonyl]-2,2-dimethyl-4-piperidyl]amino]phenyl]-3-(carboxymethoxy)-4-chloro-thiophene-2-carboxylic acid (A-65) in a similar fashion to Compound C-6, except using THF:DMF as the solvent. The residue was purified by reverse phase preparative HPLC (Column: SunFire; Mobile phase: 0.1% TFA in water/MeCN). LCMS (ES+): m/z 992.0 [M+H]⁺. ¹H NMR (400 MHz, DMSO-d₆) δ 10.99 (s, 1H), 10.17 (s, 1H), 7.68 (s, 1H), 7.66-7.61 (m, 1H), 7.51-7.44 (m, 1H), 7.36-7.29 (m, 2H), 7.24-7.08 (m, 4H), 6.86 (s, 1H), 6.79 (d, J=7.6 Hz, 1H), 6.72-6.66 (m, 1H), 5.15-5.04 (m, 1H), 4.90 (s, 2H), 4.79 (s, 2H), 4.49-4.35 (m, 4H), 4.33-4.22 (m, 2H), 4.15-3.99 (m, 2H), 3.20-3.07 (m, 4H), 2.96-2.84 (m, 2H), 2.04-1.94 (m, 1H), 1.93-1.83 (m, 1H), 1.82-1.73 (m, 1H), 1.46 (s, 3H), 1.42-1.33 (m, 4H), 1.16-1.03 (m, 1H).

### Example 75

### 3-(carboxymethoxy)-4-chloro-5-[3-[[1-[[3-[[1-[2-1 [2-(2,6-dioxo-3-piperidyl)-1-oxo-isoindolin-4-yl]amino]acetyl]azetidine-3-carbonyl]amino]-2-fluoro-phenyl]methylsulfonyl]-4-piperidyl]amino]phenyl]thiophene-2-carboxylic acid (75)

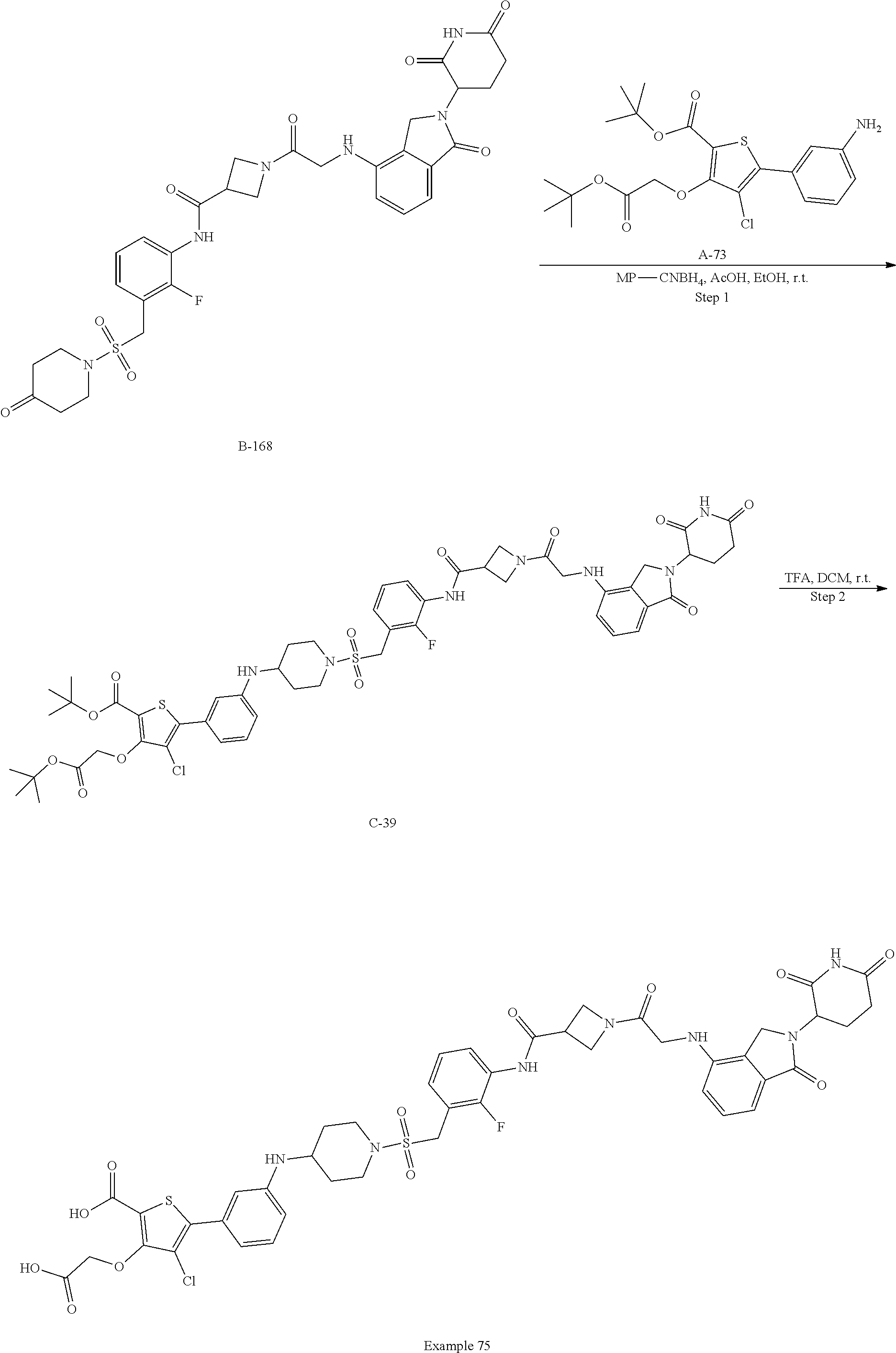

### Step 1: tert-butyl 3-(2-tert-butoxy-2-oxo-ethoxy)-4-chloro-5-[3-[[1-[[3-[[1-[2-[[2-(2,6-dioxo-3-piperidyl)-1-oxo-isoindolin-4-yl]amino]acetyl]azetidine-3-carbonyl]amino]-2-fluoro-phenyl]methylsulfonyl]-4-piperidyl]amino]phenyl]thiophene-2-carboxylate (C-39)

Tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-2-fluoro-phenyl]methylsulfonyl]-4-piperidyl]amino]phenyl]thiophene-2-carboxylate (C-39.70 mg, 0.572 mmol, 30% yield, TFA salt) was synthesized from 1-[2-[[2-(2,6-dioxo-3-piperidyl)-1-oxo-isoindolin-4-yl]amino]acetyl]-N-[2-fluoro-3-[(4-oxo-1-piperidyl)sulfonylmethyl]phenyl]azetidine-3-carboxamide (B-168) and tert-butyl 5-(3-aminophenyl)-3-(2-tert-butoxy-2-oxo-ethoxy)-4-chloro-thiophene-2-carboxylate (A-73) in a similar fashion to Compound C-38. The residue was purified by reverse phase HPLC using method: Atlantis C18 column (4.6×250 mm, 5 μm) with Solvent A: 0.1% TFA in water; Solvent B: Acetonitrile; Flow rate: 17 mL/min; R.T.: 9.6 min. LCMS (ESI): m/z 1090.1 [M−H]⁻.

### Step 2: 3-(carboxymethoxy)-4-chloro-5-[3-[[1-[[3-[[1-[2-[[2-(2,6-dioxo-3-piperidyl)-1-oxo-isoindolin-4-yl]amino]acetyl]azetidine-3-carbonyl]amino]-2-fluoro-phenyl]methylsulfonyl]-4-piperidyl]amino]phenyl]thiophene-2-carboxylic acid (75)

3-(carboxymethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-2-fluoro-phenyl]methylsulfonyl]-4-piperidyl]amino]phenyl]thiophene-2-carboxylic acid (75, 27 mg, 0.024 mmol, 42% yield, TFA salt) was synthesized from tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-2-fluoro-phenyl]methylsulfonyl]-4-piperidyl]amino]phenyl]thiophene-2-carboxylate (C-39) in a similar fashion to Compound C-1, except using 217 eq. of TFA. The residue was purified by Atlantis C18 column (4.6×250 mm, 5 μm) with Solvent A: 0.1% TFA in water; Solvent B: Acetonitrile; Flow rate: 18 mL/min; R.T.: 7.8 min. LCMS (ESI): m/z 980.0 [M+H]⁺. ¹H NMR (400 MHz, DMSO-d₆) δ 11.01 (s, 1H), 10.01 (s, 1H), 7.93 (t, J=6.8 Hz, 1H), 7.29 (t, J=7.8 Hz, 1H), 7.26-7.17 (m, 2H), 6.98 (d, J=7.4 Hz, 1H), 6.89 (s, 1H), 6.82 (d, J=7.6 Hz, 1H), 6.75-6.70 (m, 1H), 6.67 (d, J=8.0 Hz, 1H), 5.11 (dd, J=13.3, 5.1 Hz, 1H), 4.92 (s, 2H), 4.46 (s, 2H), 4.44-4.33 (m, 2H), 4.28 (d, J=17.1 Hz, 1H), 4.16 (d, J=17.1 Hz, 1H), 4.11-3.98 (m, 3H), 3.85 (d, J=3.8 Hz, 3H), 3.56 (d, J=12.2 Hz, 2H), 3.45 (s, 1H), 3.01 (t, J=11.2 Hz, 2H), 2.96-2.86 (m, 1H), 2.69-2.57 (m, 2H), 2.37-2.27 (m, 1H), 2.06-1.93 (m, 3H), 1.45-1.30 (m, 2H).

### Example 76

### 3-(carboxymethoxy)-4-chloro-5-[3-[[(4S)-1-[[3-[[4-[[2-(2,6-dioxo-3-piperidyl)-1-oxo-isoindolin-S-yl]oxypiperidine-1-carbonyl]amino]phenyl]methylsulfonyl]-2,2-dimethyl-4-piperidyl]amino]phenyl]thiophene-2-carboxylic acid (76)

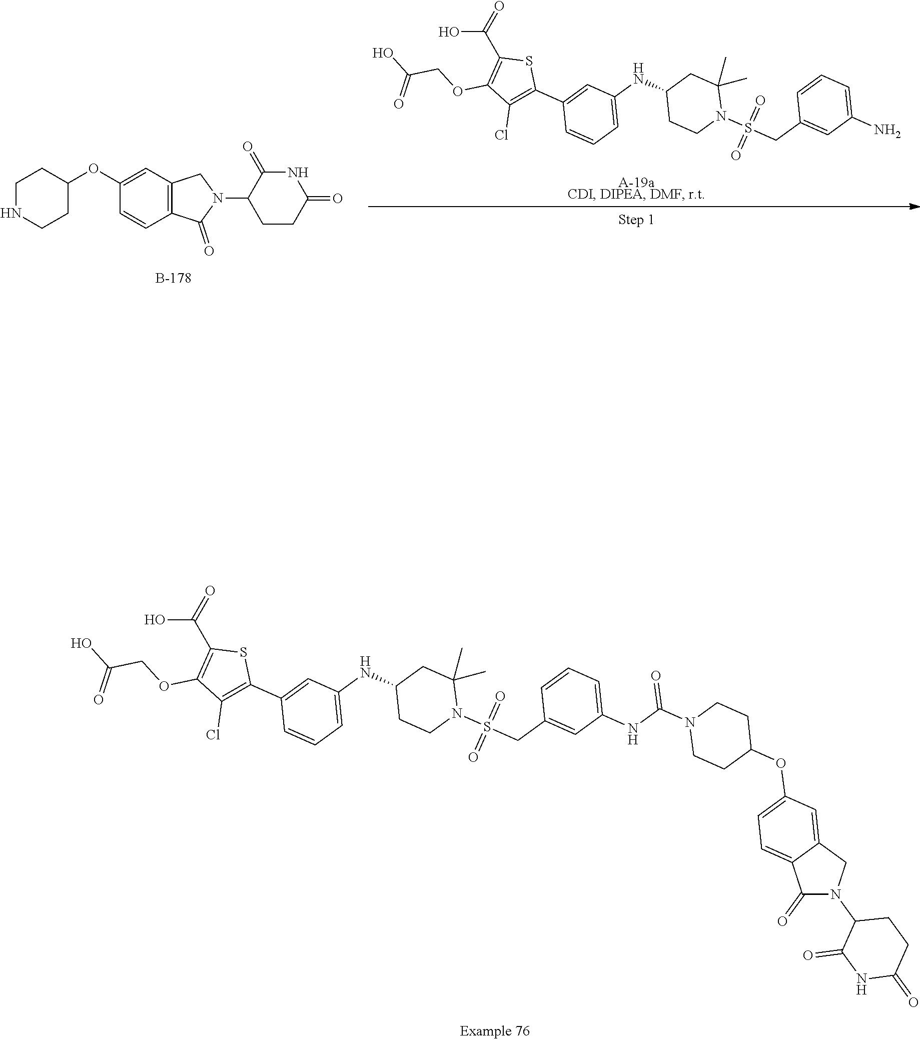 [SMILES: O=C1CCC(N2Cc3cc(OC4CCNCC4)ccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; "A-19aCDI, DIPEA, DMF, r.t." ; "Step 1" ; "B-178" ; CC1(C)C[C@@H](Nc2cccc(-c3sc(C(=O)O)c(OCC(=O)O)c3Cl)c2)CCN1S(=O)(=O)Cc1cccc(NC(=O)N2CCC(Oc3ccc4c(c3)CN(C3CCC(=O)NC3=O)C4=O)CC2)c1]

### Step 1: 3-(carboxymethoxy)-4-chloro-5-[3-[[(4S)-1-[3-[4-[[2-(2,6-dioxo-3-piperidyl)-1-oxo-isoindolin-5-yl]oxypiperidine-1-carbonyl]amino]phenyl]methylsulfonyl]-2,2-dimethyl-4-piperidyl]amino]phenyl]thiophene-2-carboxylic acid (76)

3-(carboxymethoxy)-4-chloro-5-[3-[[(4S)-1-[[3-[[4-[[2-(2,6-dioxo-3-piperidyl)-1-oxo-isoindolin-5-yl]oxypiperidine-1-carbonyl]amino]phenyl]methylsulfonyl]-2,2-dimethyl-4-piperidyl]amino]phenyl]thiophene-2-carboxylic acid (76, 5.2 mg, 4.63 μmol, 6% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[1-oxo-5-(4-piperidyloxy)isoindolin-2-yl]piperidine-2,6-dione (B-178) in a similar fashion to Example 23, except using excess DIPEA as base and DMF as the solvent. The residue was purified by reverse phase prep HPLC [Purification method: Column: SunFire C18 (19×150 mm), 5.0 μm, Mobile phase A: 0.1% TFA in water and mobile phase B: Acetonitrile]. LCMS (ES+): m/z 977.5 [M+H]⁺. ¹H NMR (400 MHz, DMSO-d6): δ 10.98 (s, 1H), 8.67 (s, 1H), 7.63 (d, J=8.40 Hz, 1H), 7.55 (s, 1H), 7.45 (d, J=8.40 Hz, 1H), 7.26-7.17 (m, 3H), 7.11-7.08 (m, 1H), 7.00-6.86 (m, 2H), 6.80 (d, J=8.00 Hz, 1H), 5.90-5.70 (m, 1H), 5.15-5.03 (m, 1H), 4.89 (s, 2H), 4.80-4.70 (m, 1H), 4.37-4.25 (m, 4H), 4.20-3.80 (m, 3H), 3.55-3.45 (m, 2H), 3.20-3.00 (m, 2H), 2.96-2.88 (m, 3H), 2.00-1.99 (m, 3H), 1.90-1.76 (m, 2H), 1.63-1.61 (m, 2H), 1.48-1.39 (m, 8H), 1.26-1.10 (m, 2H).

### Example 77

### 3-(carboxymethoxy)-4-chloro-5-[3-[[1-[[3-[1-[2-[[2-(2,6-dioxo-3-piperidyl)-1-oxo-isoindolin-4-yl]amino]acetyl]piperidine-4-carbonyl]amino]-4-fluoro-phenyl]methylsulfonyl]-4-piperidyl]amino]phenyl]thiophene-2-carboxylic acid (77)

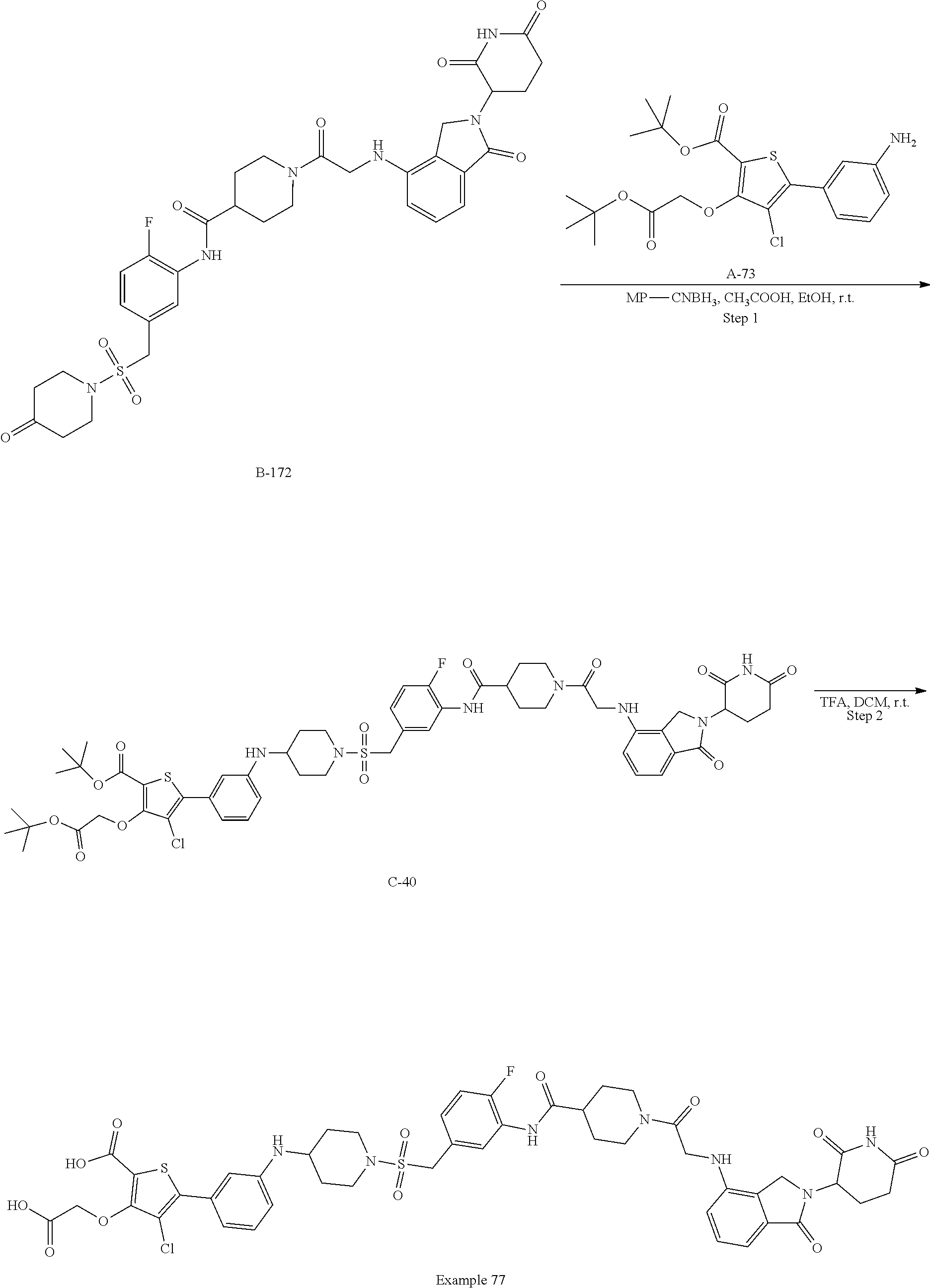

### Step 1: tert-butyl 3-(2-tert-butoxy-2-oxo-ethoxy)-4-chloro-5-[3-[[1-[[3-[[1-[2-[[2-(2,6-dioxo-3-piperidyl)-1-oxo-isoindolin-4-yl]amino]acetyl]piperidine-4-carbonyl]amino]-4-fluoro-phenyl]methylsulfonyl]-4-piperidyl]amino]phenyl]thiophene-2-carboxylate (C-40)

tert-Butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]piperidine-4-carbonyl]amino]-4-fluoro-phenyl]methylsulfonyl]-4-piperidyl]amino]phenyl]thiophene-2-carboxy late (C-40, 120 mg, 0.044 mmol, 51% yield, 45% purity) was synthesized from 1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]piperidine-4-carboxamide (B-172) and tert-butyl 5-(3-aminophenyl)-3-(2-tert-butoxy-2-oxo-ethoxy)-4-chloro-thiophene-2-carboxylate (A-73) in a similar fashion to Compound C-38. LCMS (ESI); m/z 1120.2 [M+H]⁺.

### Step 2: 3-(carboxymethoxy)-4-chloro-5-[3-[[1-[[3-[1-[2-[[2-(2,6-dioxo-3-piperidyl)-1-oxo-isoindolin-4-yl]amino]acetyl]piperidine-4-carbonyl]amino]-4-fluoro-phenyl]methylsulfonyl]-4-piperidyl]amino]phenyl]thiophene-2-carboxylic acid (77)

3-(carboxymethoxy)-4-chloro-5-[3-[[1-[[3-[1-[2-[[2-(2,6-dioxo-3-piperidyl)-1-oxo-isoindolin-4-yl]amino]acetyl]piperidine-4-carbonyl]amino]-4-fluoro-phenyl]methylsulfonyl]-4-piperidyl]amino]phenyl]thiophene-2-carboxylic acid (77, 30 mg, 0.026 mmol, 54% yield) was synthesized from tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]piperidine-4-carbonyl]amino]-4-fluoro-phenyl]methylsulfonyl]-4-piperidyl]amino]phenyl]thiophene-2-carboxy late (C-40) in a similar fashion to Compound C-1, except using 130 eq. of TFA. The residue was purified by reverse phase HPLC using the method: Column: XSelect (150×19 mm), 5 μm; Mobile phase A: 0.1% TFA in MQ-water; B: Acetonitrile; Flow rate: 15 mL/min. LCMS (ESI): m/z 1008.0 [M+H]⁺. ¹H NMR (400 MHz, DMSO-d₆) δ 11.00 (d, J=3.4 Hz, 1H), 9.82 (s, 1H), 7.93 (d, J=7.4 Hz, 1H), 7.32-7.24 (m, 2H), 7.23-7.16 (m, 2H), 7.01-6.95 (m, 1H), 6.88 (s, 1H), 6.84-6.74 (m, 2H), 6.71 (d, J=8.5 Hz, 1H), 5.14-5.07 (m, 1H), 4.93-4.90 (m, 2H), 4.40 (s, 3H), 4.30 (d, J=17.2 Hz, 1H), 4.22-4.14 (m, 1H), 4.08-3.97 (m, 3H), 3.15-3.06 (m, 2H), 3.01-2.85 (m, 4H), 2.83-2.74 (m, 1H), 2.73-2.57 (m, 4H), 2.40-2.34 (m, 2H), 2.07-1.81 (m, 5H), 1.71-1.57 (m, 1H), 1.55-1.42 (m, 1H), 1.41-1.26 (m, 2H).

### Example 78

### 3-(carboxymethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-4-piperidyl]amino]phenyl]thiophene-2-carboxylic acid (78)

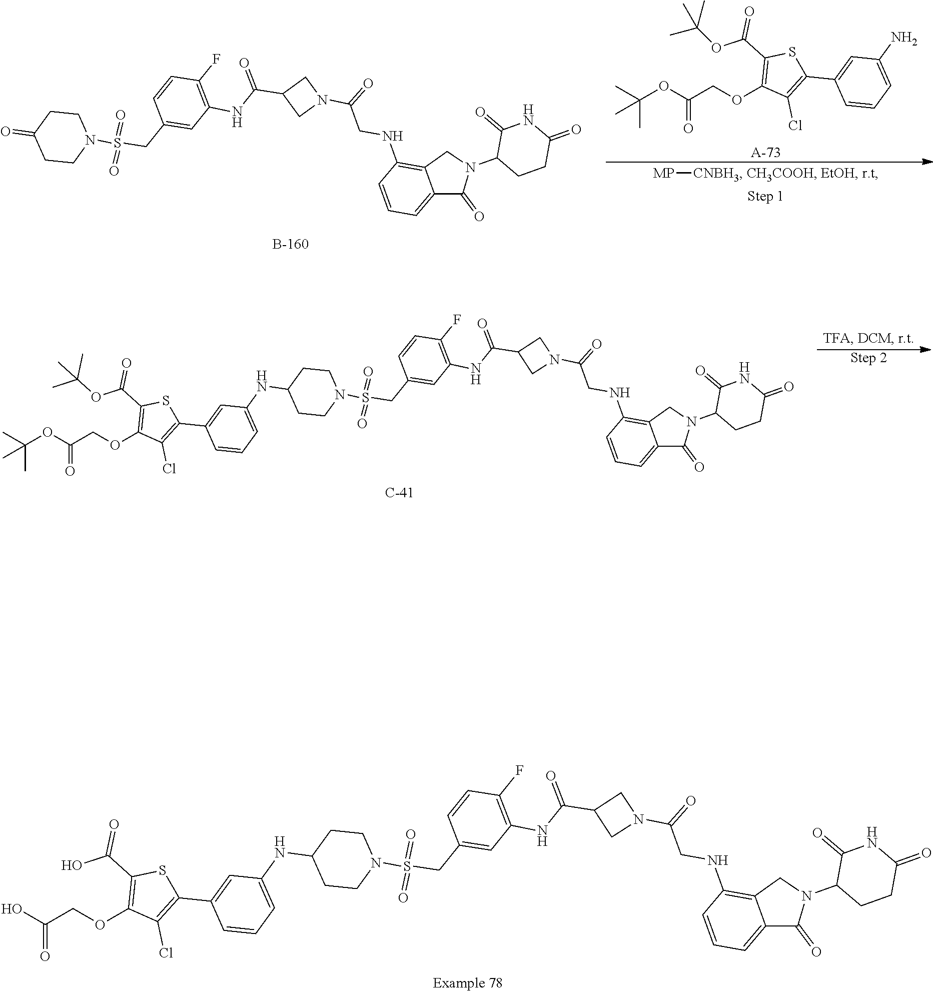

### Step 1: tert-butyl 3-(2-tert-butoxy-2-oxo-ethoxy)-4-chloro-5-[3-[[1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-4-piperidyl]amino]phenyl]thiophene-2-carboxylate (C-41)

tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-4-piperidyl]amino]phenyl]thiophene-2-carboxylate (C-41, 100 mg, 24.53 μmol, 33% yield, 30% purity) was synthesized from 1-[2-[[2-(2,6-dioxo-3-piperidyl)-1-oxo-isoindolin-4-yl]amino]acetyl]-N-[2-fluoro-5-[(4-oxo-1-piperidyl)sulfonylmethyl]phenyl]azetidine-3-carboxamide (B-160) and tert-butyl 5-(3-aminophenyl)-3-(2-tert-butoxy-2-oxo-ethoxy)-4-chloro-thiophene-2-carboxylate (A-73) in a similar fashion to Compound C-38. LCMS (ESI): m/z 1114.2 [M+Na]⁺

### Step 2: 3-(carboxymethoxy)-4-chloro-5-[3-[[1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-4-piperidyl]amino]phenyl]thiophene-2-carboxylic acid (78)

3-(carboxymethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-4-piperidyl]amino]phenyl]thiophene-2-carboxylic acid (78, 10 mg, 8.96 μmol, 34% yield) was synthesized from tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-4-piperidyl]amino]phenyl]thiophene-2-carboxylate (C-41) in a similar fashion to Compound C-1, except using 215 eq. of TFA. The residue was purified by reverse phase column chromatography using the method: Column: XSelect (150×19 mm), 5 μm; Mobile phase A: 0.1% TFA in MQ-water; Mobile phase B: Acetonitrile. LCMS (ESI): m/z 980.0 [M+H]⁺. ¹H NMR (400 MHz, DMSO-d₆) δ 11.00 (s, 1H), 9.99 (s, 1H), 8.00 (d, J=7.5 Hz, 1H), 7.29 (t, J=7.9 Hz, 2H), 7.23-7.16 (m, 2H), 7.01-6.94 (m, 1H), 6.88 (s, 1H), 6.81 (d, J=7.5 Hz, 1H), 6.74-6.69 (m, 1H), 6.66 (d, J=8.8 Hz, 1H), 5.16-5.06 (m, 1H), 4.91 (d, J=3.2 Hz, 2H), 4.40 (s, 3H), 4.37-4.32 (m, 4H), 4.27 (d, J=17.2 Hz, 2H), 4.20-4.11 (m, 2H), 4.10-3.97 (m, 3H), 3.84 (d, J=4.9 Hz, 2H), 3.00-2.87 (m, 2H), 2.68-2.61 (m, 2H), 2.04-1.89 (m, 4H), 1.42-1.29 (m, 2H).

### Example 79

### 3-(Carboxymethoxy)-4-chloro-5-[3-[[(4S)-1-[3-[3-[[1-(2,6-dioxo-3-piperidyl)-3-methyl-2-oxo-benzimidazol-S-yl]amino]azetidine-1-carbonyl]amino]phenyl]methylsulfonyl]-2,2-dimethyl-4-piperidyl]amino]phenyl]thiophene-2-carboxylic acid (79)

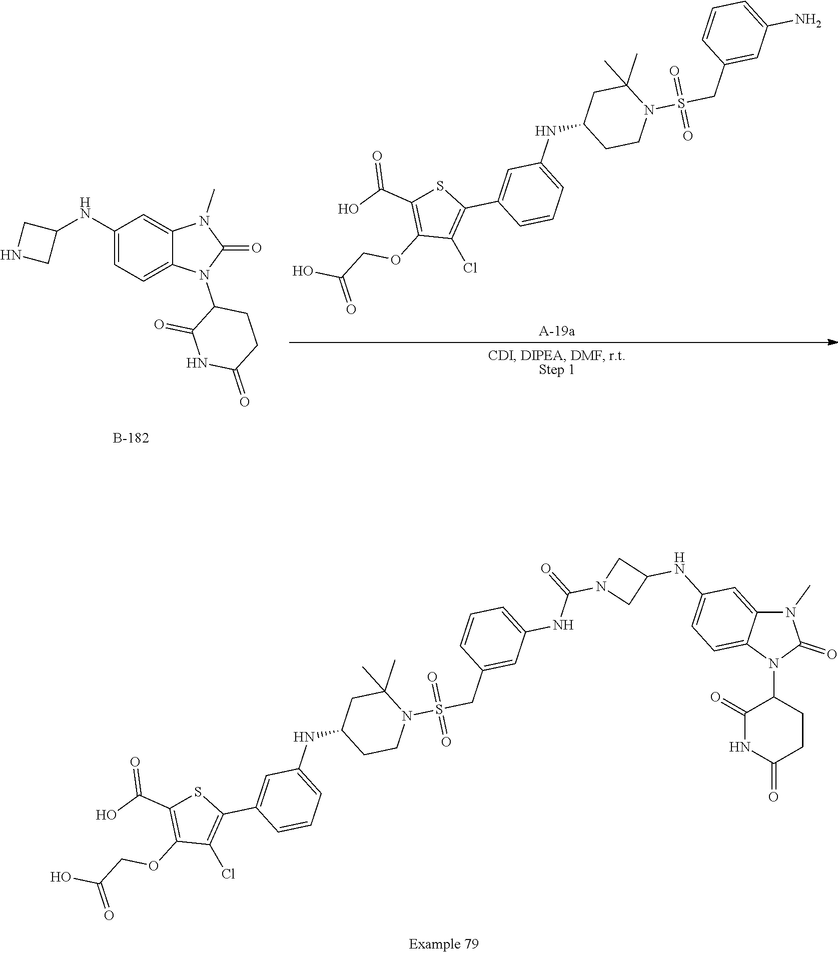 [SMILES: Cn1c(=O)n(C2CCC(=O)NC2=O)c2ccc(NC3CNC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; "A-19a" ; "CDI, DIPEA, DMF, r.t.Step 1" ; Cn1c(=O)n(C2CCC(=O)NC2=O)c2ccc(NC3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21]

### Step 1: 3-(carboxymethoxy)-4-chloro-5-[3-[[(4S)-1-[[3-[3-[[1-(2,6-dioxo-3-piperidyl)-3-methyl-2-oxo-benzimidazol-5-yl]amino]azetidine-1-carbonyl]amino]phenyl]methylsulfonyl]-2,2-dimethyl-4-piperidyl]amino]phenyl]thiophene-2-carboxylic acid (79)

3-(carboxymethoxy)-4-chloro-5-[3-[[(4S)-1-[3-[3-[[1-(2,6-dioxo-3-piperidyl)-3-methyl-2-oxo-benzimidazol-5-yl]amino]azetidine-1-carbonyl]amino]phenyl]methylsulfonyl]-2,2-dimethyl-4-piperidyl]amino]phenyl]thiophene-2-carboxylic acid (79, 10 mg, 0.00913 mmol, 9% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(azetidin-3-yl)amino)-3-methyl-2-oxo-benzimidazol-1-yl]piperidine-2,6-dione (B-182) in a similar fashion to Example 23, except using excess DIPEA as base and DMF as the solvent. The residue was purified by Reverse Phase Prep HPLC (XSelect (150×19 mm, 5 μm) with Solvent A: 0.1% TFA in water; Solvent B: Acetonitrile; Flow rate: 15 mL/min; RT=12 min. LCMS (ESI): m/z 963.0 [M+H]⁺. ¹H NMR (400 MHz, DMSO-d₆) δ 11.05 (s, 1H), 8.57 (s, 1H), 7.58 (s, 1H), 7.52 (d, J=8.2 Hz, 1H), 7.24 (t, J=7.9 Hz, 1H), 7.18 (t, J=7.9 Hz, 1H), 6.97 (d, J=7.7 Hz, 1H), 6.89-6.84 (m, 2H), 6.80 (d, J=7.6 Hz, 1H), 6.72-6.65 (m, 1H), 6.40-6.35 (m, 1H), 6.28-6.21 (m, 1H), 5.25 (dd, J=12.8, 5.3 Hz, 1H), 4.91 (s, 2H), 4.40-4.29 (m, 3H), 4.28-4.17 (m, 2H), 3.79-3.71 (m, 3H), 3.27 (s, 3H), 3.12 (t, J=12.6 Hz, 2H), 2.95-2.81 (m, 1H), 2.64-2.56 (m, 2H), 2.03-1.93 (m, 1H), 1.91-1.82 (m, 1H), 1.82-1.73 (m, 1H), 1.46 (s, 3H), 1.44-1.36 (m, 4H), 1.19-1.04 (m, 1H).

### Example 80

### 3-(Carboxymethoxy)-4-chloro-5-[3-[[(4S)-1-[[3-[[4-[1-(2,6-dioxo-3-piperidyl)-3-methyl-2-oxo-benzimidazol-5-yl]piperazine-1-carbonyl]amino]phenyl]methylsulfonyl]-2,2-dimethyl-4-piperidyl]amino]phenyl]thiophene-2-carboxylic acid (80)

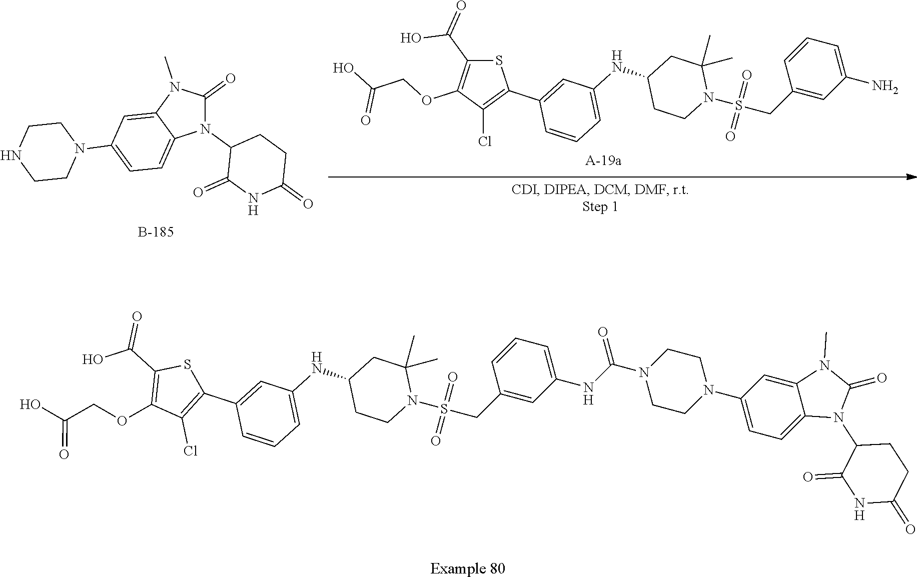 [SMILES: Cn1c(=O)n(C2CCC(=O)NC2=O)c2ccc(N3CCNCC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(N3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperazine-1-carbonyl]amino]phenyl]methylsulfonyl]-2,2-dimethyl-4-piperidyl]amino]phenyl]thiophene-2-carboxylic acid (80)

3-(carboxymethoxy)-4-chloro-5-[3-[[(4S)-1-[[3-[[4-[1-(2,6-dioxo-3-piperidyl)-3-methyl-2-oxo-benzimidazol-5-yl]piperazine-1-carbonyl]amino]phenyl]methylsulfonyl]-2,2-dimethyl-4-piperidyl]amino]phenyl]thiophene-2-carboxylic acid (80, 5.1 mg, 0.0046 mmol, 4% yield; TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-(3-methyl-2-oxo-5-piperazin-1-yl-benzimidazol-1-yl)piperidine-2,6-dione (B-185) in a similar fashion to Example 23, except using excess DIPEA as base and DMF:DCM as the solvent. The residue was purified by reverse phase HPLC following the method: Column: XSelect C18 (19×150 mm, 5 μm); Mobile phase A: 0.1% TFA in water and B: Acetonitrile; Flow rate=15 mL/min. LCMS (ESI): m/z 977.20 [M+H]⁺. ¹H NMR (400 MHz, DMSO-d₆) δ 11.07 (s, 1H), 8.71 (s, 1H), 7.56 (s, 1H), 7.46 (d, J=8.4 Hz, 1H), 7.27-7.17 (m, 2H), 7.01-6.96 (m, 2H), 6.90 (d, J=1.6 Hz, 1H), 6.86 (s, 1H), 6.80 (d, J=7.5 Hz, 1H), 6.68 (d, J=8.5 Hz, 2H), 5.32-5.27 (m, 1H), 4.90 (s, 2H), 4.36 (d, J=14.0 Hz, 2H), 4.24 (d, J=13.6 Hz, 1H), 3.65-3.63 (m, 6H), 3.17-3.12 (m, 7H), 2.58-2.52 (m, 4H), 2.01-1.98 (m, 2H), 1.86-1.76 (m, 3H) and 1.48-1.39 (m, 6H).

### Example 81

### 3-(carboxymethoxy)-4-chloro-5-[3-[[(4S)-1-[[3-[[4-[[1-(2,6-dioxo-3-piperidyl)-3-methyl-2-oxo-benzimidazol-5-yl]amino]piperidine-1-carbonyl]amino]phenyl]methylsulfonyl]-2,2-dimethyl-4-piperidyl]amino]phenyl]thiophene-2-carboxylic acid (81)

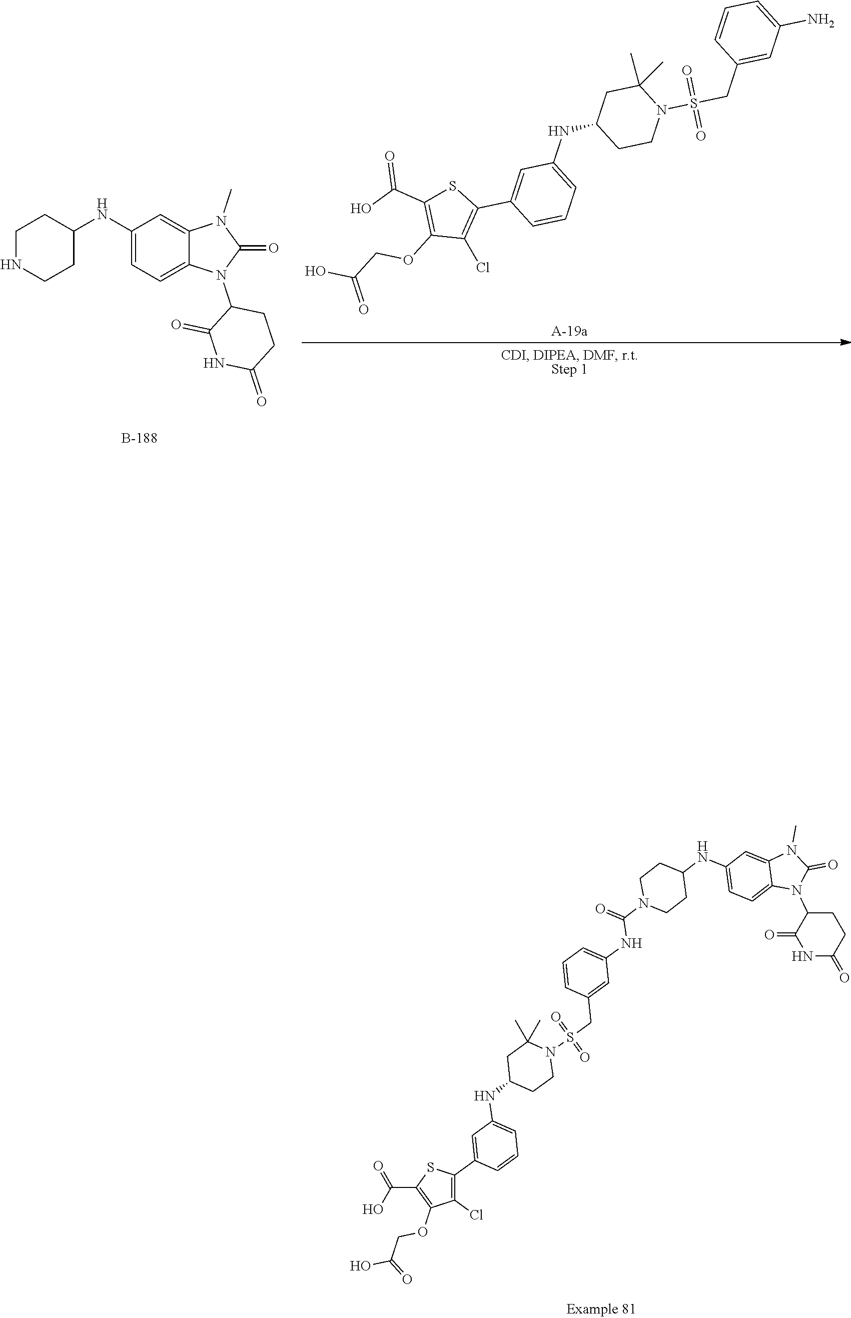 [SMILES: Cn1c(=O)n(C2CCC(=O)NC2=O)c2ccc(NC3CCNCC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; "A-19a" ; "CDI, DIPEA, DMF, r.t.Step 1" ; Cn1c(=O)n(C2CCC(=O)NC2=O)c2ccc(NC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[4-[[1-(2,6-dioxo-3-piperidyl)-3-methyl-2-oxo-benzimidazol-5-yl]amino]piperidine-1-carbonyl]amino]phenyl]methylsulfonyl]-2,2-dimethyl-4-piperidyl]amino]phenyl]thiophene-2-carboxylic acid (81)

3-(carboxymethoxy)-4-chloro-5-[3-[[(4S)-1-[[3-[[4-[1-(2,6-dioxo-3-piperidyl)-3-methyl-2-oxo-benzimidazol-5-yl]amino]piperidine-1-carbonyl]amino]phenyl]methylsulfonyl]-2,2-dimethyl-4-piperidyl]amino]phenyl]thiophene-2-carboxylic acid (81, 7 mg, 0.006 mmol, 6% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-methyl-2-oxo-5-(4-piperidyl]amino)benzimidazol-1-yl]piperidine-2,6-dione (B-188) in a similar fashion to Example 23, except using excess DIPEA as base and DMF as the solvent. The residue was purified by reverse phase HPLC following the method: Column: XSelect (19×150 mm, 5 μm) with Solvent A: 0.1% TFA in water; Solvent B: Acetonitrile; Flow rate: 13 mL/min; RT=14 min. LCMS (ESI): m/z 991.0 [M+H]⁺. ¹H NMR (400 MHz, DMSO-d₆) δ 11.11 (s, 1H), 8.67 (s, 1H), 7.53 (s, 1H), 7.45 (d, J=8.1 Hz, 1H), 7.27-7.18 (m, 4H), 6.99 (d, J=7.7 Hz, 2H), 6.87 (s, 1H), 6.81 (d, J=7.3 Hz, 1H), 6.69 (d, J=8.1 Hz, 1H), 5.35-5.32 (m, 1H), 4.92 (s, 2H), 4.36 (d, J=13.7 Hz, 1H), 4.25 (d, J=13.9 Hz, 1H), 4.14 (d, J=12.8 Hz, 2H), 3.33-3.06 (m, 6H), 2.97-2.81 (m, 5H), 2.68-2.59 (m, 2H), 2.06-1.76 (m, 7H), 1.49 (s, 3H), 1.48-1.41 (m, 3H) and 1.40 (s, 3H).

### Example 82

### 3-(carboxymethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (82)

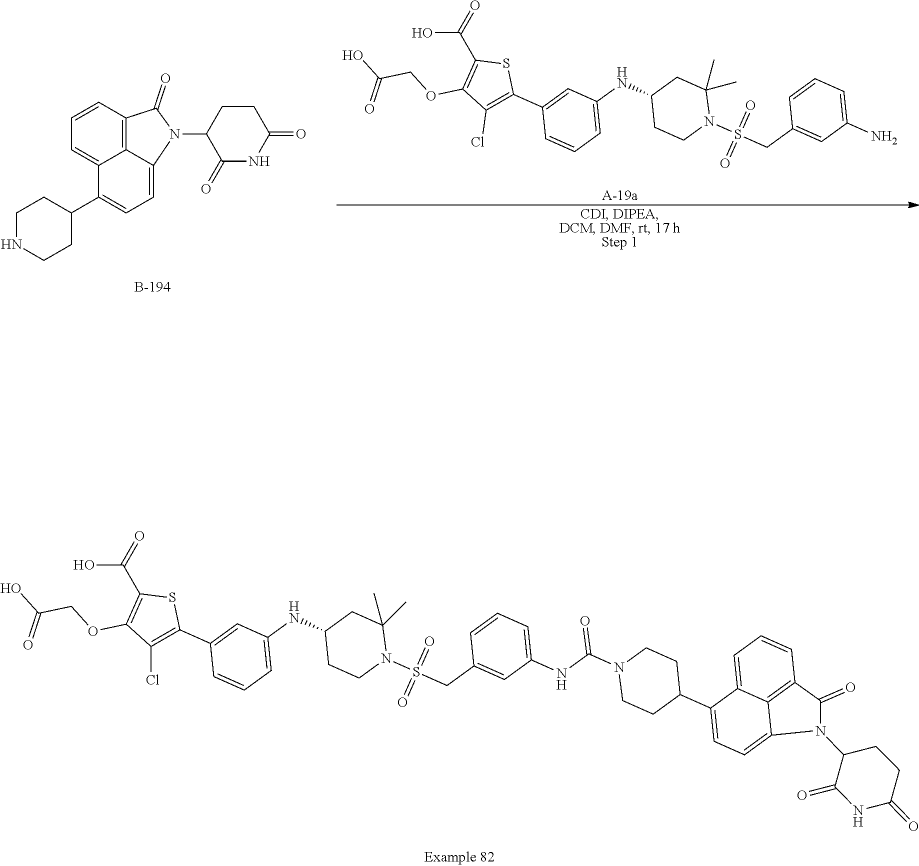 [SMILES: O=C1CCC(N2C(=O)c3cccc4c(C5CCNCC5)ccc2c34)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; "A-19a" ; "CDI, DIPEA,DCM, DMF, rt, 17 hStep 1" ; "B-194" ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1]

### Step 1: 3-(carboxymethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (82)

3-(carboxymethoxy)-4-chloro-5-[3-[[(4S)-1-[3-[4-[1-(2,6-dioxo-3-piperidyl)-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (82, 7 mg, 6.19 μmol, 13% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[2-oxo-6-(4-piperidyl)benzo[cd]indol-1-yl]piperidine-2,6-dione (B-194) in a similar fashion to Example 23, except using excess DIPEA as base and DMF:DCM as solvent. The residue was purified by reverse phase prep HPLC (Column: SunFire C-18 (150×19 mm), 5 μm; Mobile phase A: 0.1% TFA in water, Mobile phase B: MeCN). LCMS (ES+): m/z 997.1 [M+H]⁺. ¹H NMR (400 MHz, DMSO-d₆) δ 11.13 (s, 1H), 8.66 (s, 1H), 8.49 (d, J=8.4 Hz, 1H), 8.11 (d, J=7.0 Hz, 1H), 7.87 (t, J=7.6 Hz, 1H), 7.58 (s, 1H), 7.47 (d, J=8.2 Hz, 1H), 7.37 (d, J=7.5 Hz, 1H), 7.25 (t, J=7.7 Hz, 1H), 7.19 (t, J=7.9 Hz, 1H), 7.09 (d, J=7.6 Hz, 1H), 6.99 (d, J=7.7 Hz, 1H), 6.86 (s, 1H), 6.79 (d, J=7.6 Hz, 1H), 6.68 (d, J=8.3 Hz, 1H), 5.47-5.37 (m, 1H), 4.90 (s, 2H), 4.42-4.31 (m, 4H), 4.25 (d, J=13.8 Hz, 1H), 3.18-3.03 (m, 5H), 3.00-2.88 (m, 3H), 2.81-2.71 (m, 2H), 2.14-2.05 (m, 1H), 1.89 (t, J=12.0 Hz, 3H), 1.81-1.66 (m, 3H), 1.48 (s, 3H), 1.45-1.34 (m, 4H), 1.18-1.02 (m, 1H).

### Example 83

### 3-(carboxymethoxy)-4-chloro-5-[3-[[(4S)-1-[[3-[[2-[4-[1-(2,6-dioxo-3-piperidyl)-2-oxo-benzo[cd]indol-6-yl]-1-piperidyl]acetyl]amino]phenyl]methylsulfonyl]-2,2-dimethyl-4-piperidyl]amino]phenyl]thiophene-2-carboxylic acid (83)

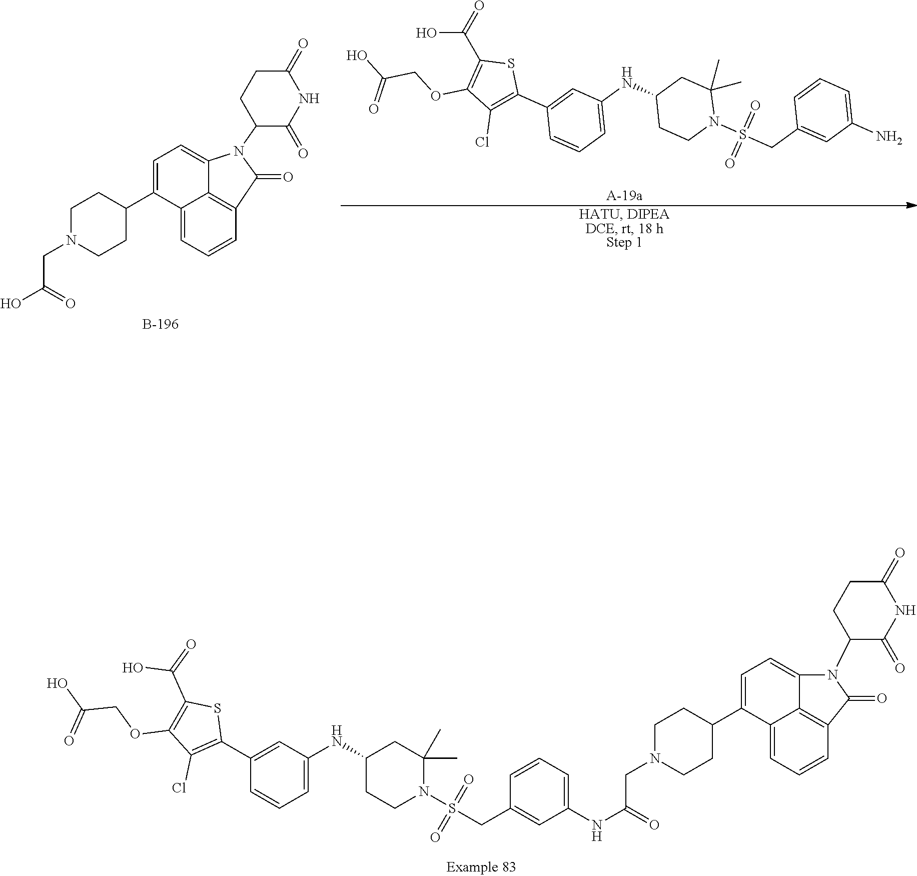 [SMILES: O=C(O)CN1CCC(c2ccc3c4c(cccc24)C(=O)N3C2CCC(=O)NC2=O)CC1 ; CC1(C)C[C@@H](Nc2cccc(-c3sc(C(=O)O)c(OCC(=O)O)c3Cl)c2)CCN1S(=O)(=O)Cc1cccc(N)c1 ; "A-19a" ; "HATU, DIPEADCE, rt, 18 hStep 1" ; "B-196" ; CC1(C)C[C@@H](Nc2cccc(-c3sc(C(=O)O)c(OCC(=O)O)c3Cl)c2)CCN1S(=O)(=O)Cc1cccc(NC(=O)CN2CCC(c3ccc4c5c(cccc35)C(=O)N4C3CCC(=O)NC3=O)CC2)c1]

### Step 1: 3-(carboxymethoxy)-4-chloro-5-[3-[[(4S)-1-[[3-[[2-[4-[1-(2,6-dioxo-3-piperidyl)-2-oxo-benzo[cd]indol-6-yl]-1-piperidyl]acetyl]amino]phenyl]methylsulfonyl]-2,2-dimethyl-4-piperidyl]amino]phenyl]thiophene-2-carboxylic acid (83)

3-(carboxymethoxy)-4-chloro-5-[3-[[(4S)-1-[[3-[[2-[4-[1-(2,6-dioxo-3-piperidyl)-2-oxo-benzo[cd]indol-6-yl]-1-piperidyl]acetyl]amino]phenyl]methylsulfonyl]-2,2-dimethyl-4-piperidyl]amino]phenyl]thiophene-2-carboxylic acid (83, 17 mg, 14.71 μmol, 15% yield) was synthesized from 2-[4-[1-(2,6-dioxo-3-piperidyl)-2-oxo-benzo[cd]indol-6-yl]-1-piperidyl]acetic acid (B-196) and 5-[3-[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1. The residue was purified by reverse phase purified by prep HPLC (Column: SunFire C18 (150×19 mm), 5 μm; Mobile phase A: 0.1% TFA in water; Mobile phase B: MeCN). LCMS (ES+): m/z 1010.9 [M+H]⁺. ¹H NMR (400 MHz, DMSO-d₆) δ 11.15 (s, 1H), 10.71 (s, 1H), 8.48 (d . . . ]=8.4 Hz, 1H), 8.13 (d, J=6.9 Hz, 1H), 7.89 (t, J=7.7 Hz, 1H), 7.71 (s, 1H), 7.62 (d, J=8.2 Hz, 1H), 7.40 (t, J=7.9 Hz, 1H), 7.35 (d, J=7.5 Hz, 1H), 7.22-7.11 (m, 3H), 6.86 (s, 1H), 6.78 (d, J=7.6 Hz, 1H), 6.68 (d, J=8.3 Hz, 1H), 5.44 (dd, J=12.7, 5.5 Hz, 1H), 4.90 (s, 2H), 4.45 (d, J=13.8 Hz, 1H), 4.33 (d, J=13.8 Hz, 1H), 4.22 (s, 2H), 3.20-3.07 (m, 2H), 3.01-2.88 (m, 2H), 2.81-2.62 (m, 3H), 2.28-2.14 (m, 2H), 2.14-2.01 (m, 3H), 1.94-1.74 (m, 2H), 1.47 (s, 3H), 1.39 (s, 3H), 1.14-1.01 (m, 1H).

### Example 84

### 3-(carboxymethoxy)-4-chloro-5-[3-[[(4S)-1-[[3-[[4-[[2-(2,6-dioxo-3-piperidyl)-1-oxo-isoindolin-4-yl]methylcarbamoyl]piperidine-1-carbonyl]amino]phenyl]methylsulfonyl]-2,2-dimethyl-4-piperidyl]amino]phenyl]thiophene-2-carboxylic acid (84)

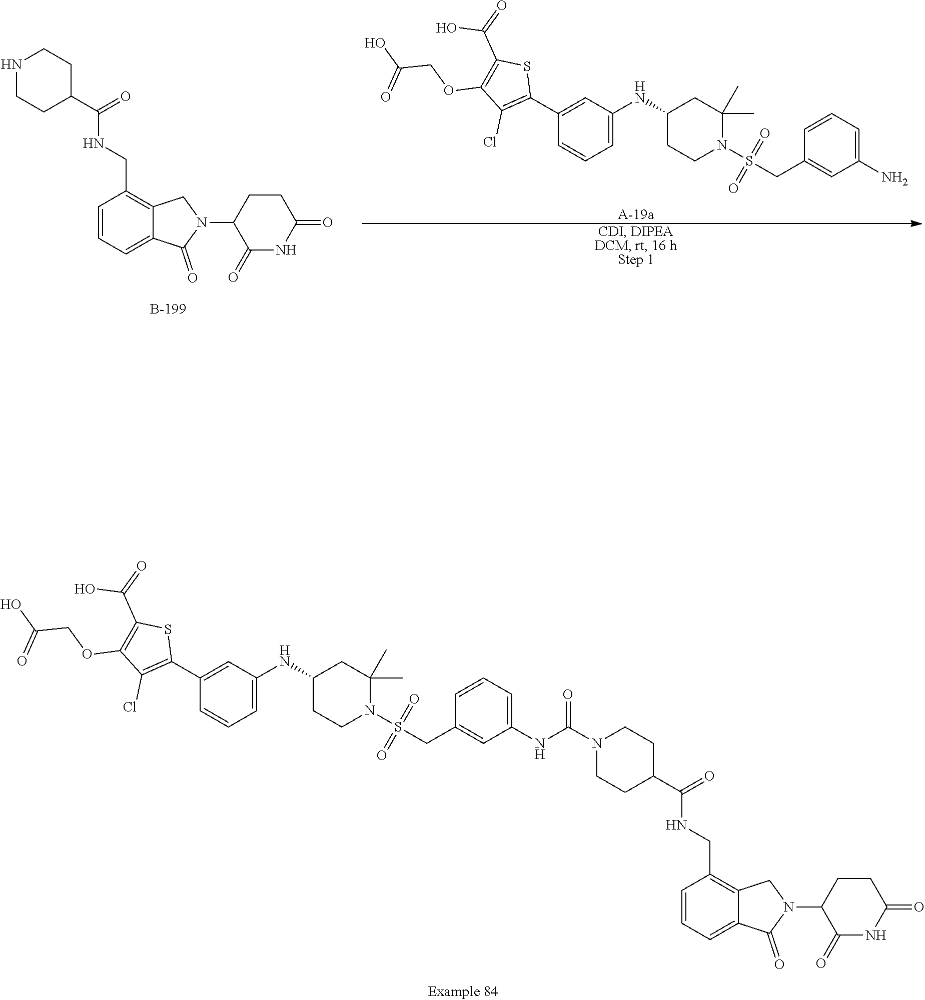 [SMILES: O=C1CCC(N2Cc3c(CNC(=O)C4CCNCC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; "A-19a" ; "CDI, DIPEADCM, rt, 16 hStep 1" ; "B-199" ; CC1(C)C[C@@H](Nc2cccc(-c3sc(C(=O)O)c(OCC(=O)O)c3Cl)c2)CCN1S(=O)(=O)Cc1cccc(NC(=O)N2CCC(C(=O)NCc3cccc4c3CN(C3CCC(=O)NC3=O)C4=O)CC2)c1]

### Step 1: 3-(carboxymethoxy)-4-chloro-5-[3-[[(4S)-1-[[3-[[4-[[2-(2,6-dioxo-3-piperidyl)-1-oxo-isoindolin-4-yl]methylcarbamoyl]piperidine-1-carbonyl]amino]phenyl]methylsulfonyl]-2,2-dimethyl-4-piperidyl]amino]phenyl]thiophene-2-carboxylic acid (84)

3-(carboxymethoxy)-4-chloro-5-[3-[[(4S)-1-[[3-[[4-[[2-(2,6-dioxo-3-piperidyl)-1-oxo-isoindolin-4-yl]methylcarbamoyl]piperidine-1-carbonyl]amino]phenyl]methylsulfonyl]-2,2-dimethyl-4-piperidyl]amino]phenyl]thiophene-2-carboxylic acid (84, 9 mg, 7.74 μmol, 12% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and N-[[2-(2,6-dioxo-3-piperidyl)-1-oxo-isoindolin-4-yl]methyl]piperidine-4-carboxamide (B-199) in a similar fashion to Example 23, except using excess DIPEA as base and DCM as the solvent. The residue was purified by reverse phase prep HPLC [Method: SunFire C18 (19×150 mm) 5 μm; Mobile phase A: 0.1% TFA in water, Mobile phase B: MeCN]. LCMS (ES+): m/z 1020.2 [M+H]⁺. ¹H NMR (400 MHz, DMSO-d₆): δ 13.45 (bs, 1H), 11.02 (s, 1H), 8.58 (s, 1H), 8.43 (t, J=5.60 Hz, 1H), 7.64-7.62 (m, 1H), 7.54-7.43 (m, 4H), 7.26-7.20 (m, 2H), 7.02-6.95 (m, 1H), 6.87-6.77 (m, 1H), 6.70-6.67 (m, 1H), 5.73-5.65 (m, 1H), 5.18-5.12 (m, 1H), 4.90 (s, 2H), 4.50 (d, J=17.20 Hz, 1H), 4.39-4.34 (m, 4H), 4.24 (d, J=14.00 Hz, 1H), 4.17-4.13 (m, 2H), 3.58-3.44 (m, 3H), 3.20-3.10 (m, 3H), 3.00-2.79 (m, 4H), 2.20-2.09 (m, 1H), 2.45-2.35 (m, 3H), 2.07-1.98 (m, 2H), 1.90-1.80 (m, 1H), 1.79-1.70 (m, 4H), 1.54-1.48 (m, 7H), 1.45-1.40 (m, 4H), 1.17-1.05 (m, 3H).

### Example 85

### 3-(carboxymethoxy)-4-chloro-5-[3-[[(4S)-1-[[3-[1-[2-[[2-(2,6-dioxo-3-piperidyl)-1-oxo-isoindolin-4-yl]amino]acetyl]-4-piperidyl]oxycarbonylamino]phenyl]methylsulfonyl]-2,2-dimethyl-4-piperidyl]amino]phenyl]thiophene-2-carboxylic acid (85)

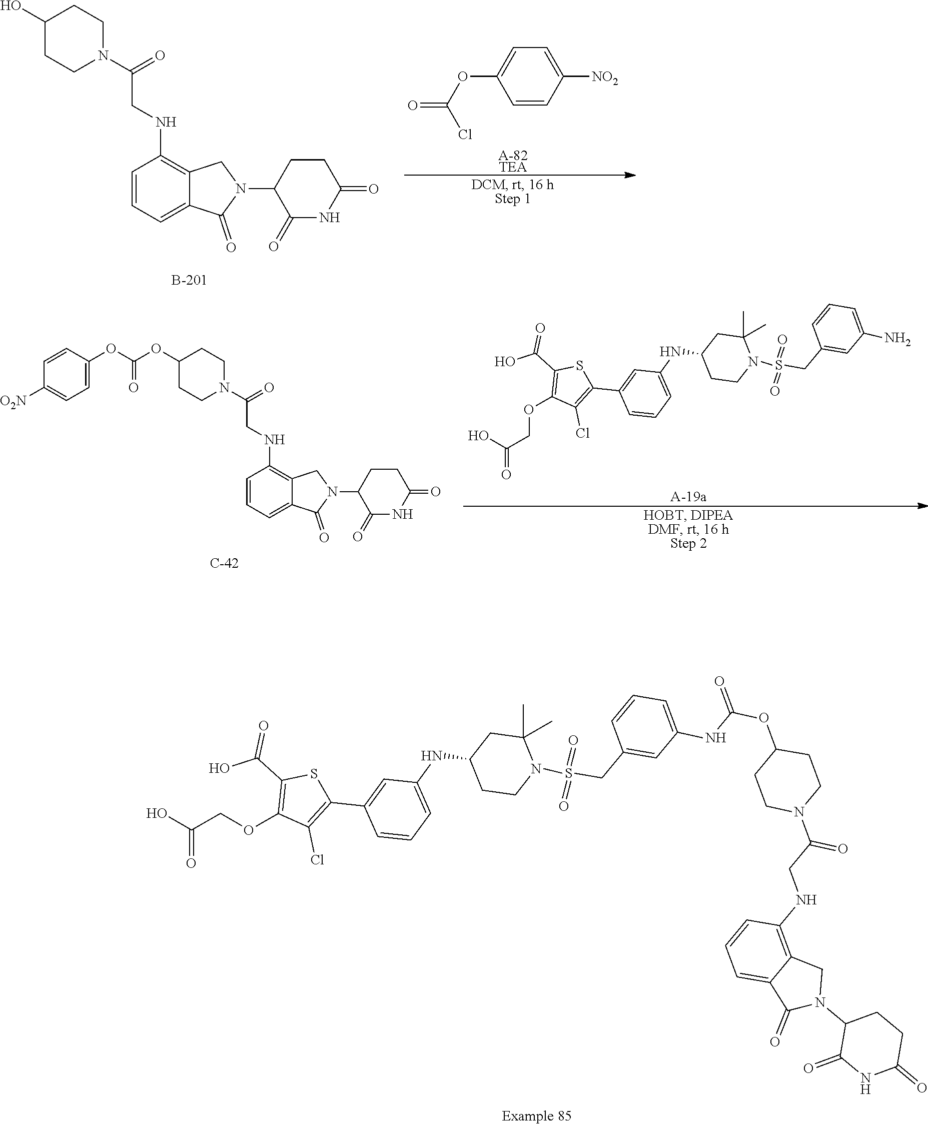

### Step 1: [1-[2-[[2-(2,6-dioxo-3-piperidyl)-1-oxo-isoindolin-4-yl]amino]acetyl]-4-piperidyl] (4-nitrophenyl)carbonate (C-42)

Into a 50 mL single neck round bottom flask containing a well-stirred solution of 3-[4-[[2-(4-hydroxy-1-piperidyl)-2-oxo-ethyl]amino]-1-oxo-isoindolin-2-yl]piperidine-2,6-dione (B-201, 170 mg, 424.55 μmol) in anhydrous DCM (3 mL) were added 4-nitrophenyl chloroformate (A-82, 256.72 mg, 1.27 mmol) and triethylamine (171.84 mg, 1.70 mmol, 236.69 μL) under nitrogen atmosphere at ambient temperature. The reaction mixture was stirred for 16 h. After completion of the reaction, volatiles were removed under reduced pressure and the residue was purified by flash silica-gel (230-400 mesh) column chromatography (0-15% MeOH in DCM) to afford [1-[2-[[2-(2,6-dioxo-3-piperidyl)-1-oxo-isoindolin-4-yl]amino]acetyl]-4-piperidyl](4-nitrophenyl)carbonate (C-42, 85 mg, 90.18 μmol, 21% yield) as a pale-yellow solid. LCMS (ES+): m/z 566.0 [M+H]⁺.

### Step 2: 3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]-4-piperidyl]oxycarbonylamino]phenyl]methylsulfonyl]-2,2-dimethyl-4-piperidyl]amino]phenyl]thiophene-2-carboxylic acid (85)

Into a 10 mL single neck round bottom flask containing a well-stirred solution of [1-[2-[[2-(2,6-dioxo-3-piperidyl)-1-oxo-isoindolin-4-yl]amino]acetyl]-4-piperidyl](4-nitrophenyl)carbonate (C-42, 85 mg, 150.30 μmol) in anhydrous DMF (1 mL) was added 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 42 mg, 69.06 μmol), HOBT (10.15 mg, 75.15 μmol) and DIPEA (58.28 mg, 450.90 μmol, 78.54 μL) under nitrogen atmosphere at ambient temperature. The reaction was stirred for 16 h. After completion of the reaction, volatiles were removed under reduced pressure and the resultant crude residue was purified using reverse phase prep HPLC [Method: Column: XBridge C18 (150×19 mm) 5 μm; Mobile phase A: 0.1% TFA in water and Mobile phase B: MeCN] to afford 3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]-4-piperidyl]oxycarbonylamino]phenyl]methylsulfonyl]-2,2-dimethyl-4-piperidyl]amino]phenyl]thiophene-2-carboxylic acid (85, 7.2 mg, 5.96 μmol, 4% yield, TFA salt) as an off-white solid. LCMS (ES+): m/z 1034.0 [M+H]⁺. ¹H NMR (400 MHz, DMSO-d₆) δ 11.00 (s, 1H), 9.73 (s, 1H), 7.59 (s, 1H), 7.43 (d, J=8.2 Hz, 1H), 7.33-7.26 (m, 2H), 7.20 (t, J=7.8 Hz, 1H), 7.03 (d, J=7.5 Hz, 1H), 6.99 (d, J=7.4 Hz, 1H), 6.88-6.85 (m, 1H), 6.83-6.76 (m, 2H), 6.72-6.66 (m, 1H), 5.11 (dd, J=13.2, 5.1 Hz, 1H), 4.91 (s, 3H), 4.41-4.13 (m, 5H), 4.09-4.04 (m, 2H), 3.91 (s, 2H), 3.83-3.75 (m, 2H), 3.15-3.06 (m, 3H), 2.98-2.86 (m, 2H), 2.64-2.56 (m, 1H), 2.42-2.36 (m, 1H), 2.06-1.83 (m, 4H), 1.83-1.75 (m, 1H), 1.68-1.58 (m, 1H), 1.55-1.42 (m, 4H), 1.38 (s, 3H), 1.16-1.03 (m, 1H).

### Example 86

### 3-(carboxymethoxy)-4-chloro-5-(3-(((4S)-1-((3-((1r,4R)-4-((2-(2,6-dioxopiperidin-3-yl)-1-oxoisoindolin-4-yl)carbamoyl)cyclohexane-1-carboxamido)benzyl)sulfonyl)-2,2-dimethylpiperidin-4-yl)amino)phenyl)thiophene-2-carboxylic acid (86)

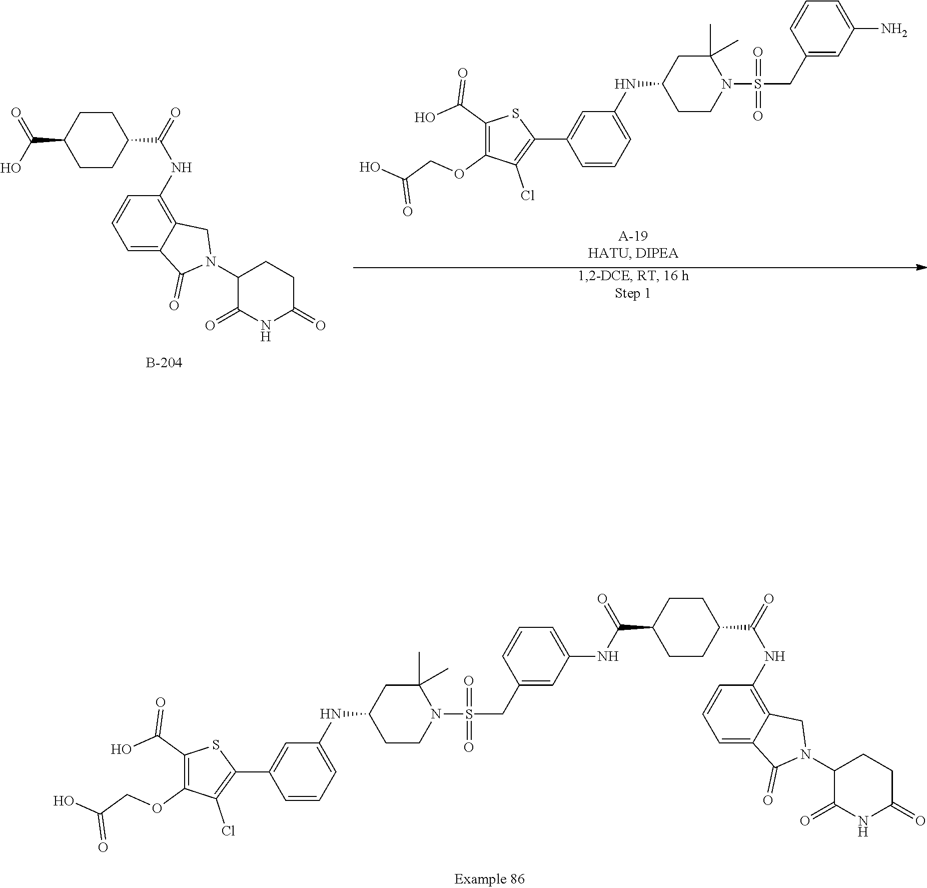 [SMILES: O=C1CCC(N2Cc3c(NC(=O)[C@H]4CC[C@H](C(=O)O)CC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@H](C(=O)Nc3cccc4c3CN(C3CCC(=O)NC3=O)C4=O)CC2)c1]

### Step 1: 3-(carboxymethoxy)-4-chloro-5-(3-(((4S)-1-((3-((1r,4R)-4-((2-(2,6-dioxopiperidin-3-yl)-1-oxoisoindolin-4-yl)carbamoyl)cyclohexane-1-carboxamido)benzyl)sulfonyl)-2,2-dimethylpiperidin-4-yl)amino)phenyl)thiophene-2-carboxylic acid (86)

3-(carboxymethoxy)-4-chloro-5-(3-(((4S)-1-((3-((1r,4R)-4-((2-(2,6-dioxopiperidin-3-yl)-1-oxoisoindolin-4-yl)carbamoyl)cyclohexane-1-carboxamido)benzyl)sulfonyl)-2,2-dimethylpiperidin-4-yl)amino)phenyl)thiophene-2-carboxylic acid (86, 15.0 mg, 13.07 μmol, 14% yield, TFA salt) was synthesized from (1r,4r)-4-((2-(2,6-dioxopiperidin-3-yl)-1-oxoisoindolin-4-yl)carbamoyl)cyclohexane-1-carboxylic acid (B-204) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1. The residue was purified by reverse phase prep HPLC [Purification method: Column: ATLANTIS C18; Mobile phase A: 0.1% TFA in water; Mobile phase B: MeCN]. LCMS (ES+): m/z 1003.0 [M+H]⁺. ¹H NMR (400 MHz, DMSO-d₆) δ 11.03 (s, 1H), 9.97 (s, 1H), 9.74 (s, 1H), 7.84 (dd, J=6.9, 2.1 Hz, 1H), 7.73-7.69 (m, 1H), 7.62-7.57 (m, 1H), 7.53-7.46 (m, 2H), 7.30 (t, J=7.9 Hz, 1H), 7.20 (t, J=7.9 Hz, 1H), 7.10-7.04 (m, 1H), 6.89-6.86 (m, 1H), 6.83-6.79 (m, 1H), 6.73-6.66 (m, 1H), 5.15 (dd, J=13.3, 5.2 Hz, 1H), 4.91 (s, 2H), 4.45-4.24 (m, 4H), 3.49-3.42 (m, 3H), 3.13 (t, J=12.6 Hz, 1H), 2.98-2.85 (m, 1H), 2.65-2.56 (m, 1H), 2.44-2.35 (m, 3H), 2.06-1.84 (m, 6H), 1.79 (d, J=12.6 Hz, 1H), 1.58-1.43 (m, 7H), 1.42-1.36 (m, 4H), 1.19-1.03 (m, 1H).

### Example 87

### 3-(carboxymethoxy)-4-chloro-5-[3-[[(4S)-1-[[3-[[4-[1-(2,6-dioxo-3-piperidyl)-3-isopropyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (87)

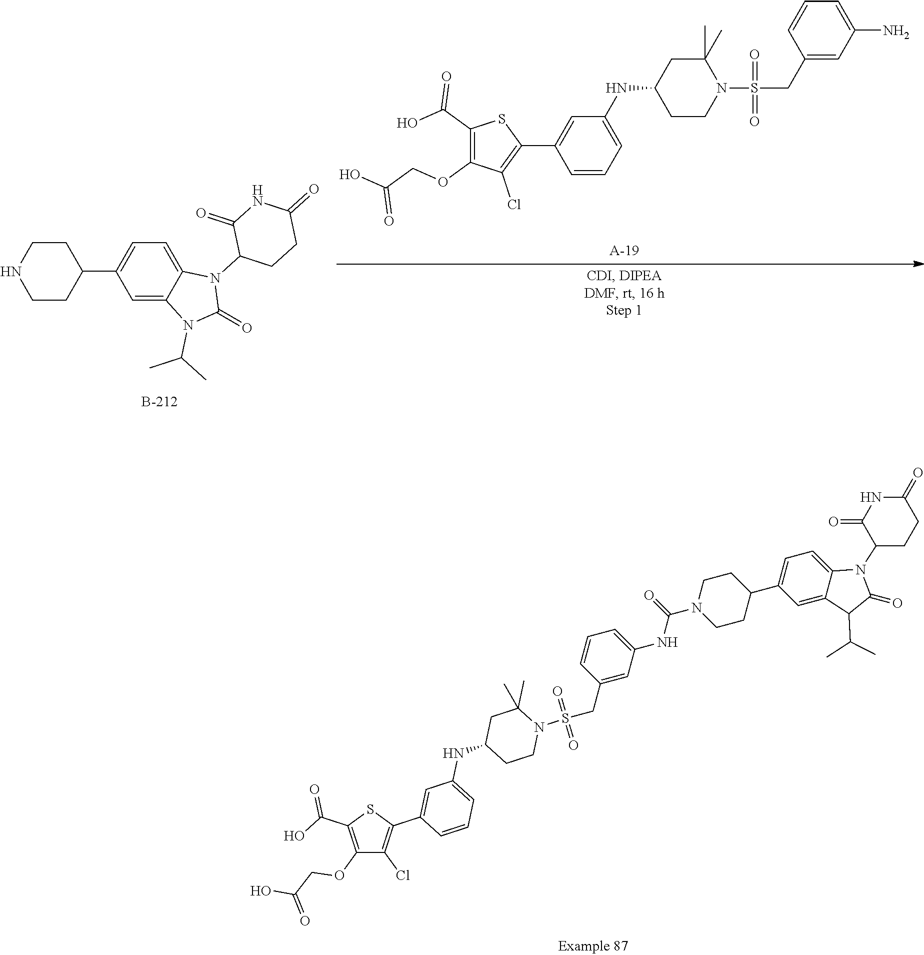 [SMILES: CC(C)n1c(=O)n(C2CCC(=O)NC2=O)c2ccc(C3CCNCC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; "A-19" ; CC(C)C1C(=O)N(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[4-[1-(2,6-dioxo-3-piperidyl)-3-isopropyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (87)

3-(carboxymethoxy)-4-chloro-5-[3-[[(4S)-1-[3-[4-[[1-(2,6-dioxo-3-piperidyl)-3-isopropyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (87, 30.36 mg, 27.06 μmol, 24% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-isopropyl-2-oxo-5-(4-piperidyl)benzimidazol-1-yl]piperidine-2,6-dione (B-212) in a similar fashion to Example 23, except using excess DIPEA as base and DMF as the solvent. The residue was purified by reverse phase preparatory HPLC (Column: XBridge (19×150 mm), 5.0 μm; Mobile phase A: 0.1% TFA in water; Mobile phase B: MeCN), LCMS [ES+]: m/z 1004.0 [M+H]⁺. ¹H NMR (400 MHz, DMSO-d6): δ 11.09 (s, 1H), 8.62 (s, 1H), 7.58 (s, 1H), 7.48 (d, J=9.60 Hz, 1H), 7.27-7.18 (m, 4H), 7.08 (s, 1H), 7.02-6.93 (m, 4H), 6.87 (s, 1H), 6.82-6.80 (m, 1H), 6.72-6.65 (m, 1H), 5.73-5.65 (m, 1H), 5.45-5.35 (m, 1H), 4.90 (s, 2H), 4.70-4.58 (m, 1H), 4.39-4.27 (m, 5H), 3.45-3.35 (m, 2H), 3.13-3.10 (m, 2H), 2.91-2.69 (m, 5H), 2.01-1.59 (m, 8H), 1.49-1.45 (m, 10H), 1.40 (s, 1H), 1.19-1.05 (m, 2H).

### Example 87a and Example 87b

### 3-(carboxymethoxy)-4-chloro-5-[3-[[(4S)-1-[3-[4-[3-isopropyl-2-oxo-1-[(3S)-2,6-dioxo-3-piperidyl]benzimidazol-5-yl]piperidine-1-carbonyl]amino]phenyl]methylsulfonyl]-2,2-dimethyl-4-piperidyl]amino]phenyl]thiophene-2-carboxylic acid (87a) and 3-(carboxymethoxy)-4-chloro-5-[3-[[(4S)-1-[[3-[[4-[3-isopropyl-2-oxo-1-[(3R)-2,6-dioxo-3-piperidyl]benzimidazol-5-yl]piperidine-1-carbonyl]amino]phenyl]methylsulfonyl]-2,2-dimethyl-4-piperidyl]amino]phenyl]thiophene-2-carboxylic acid (87b)

Configurations are arbitrarily assigned.

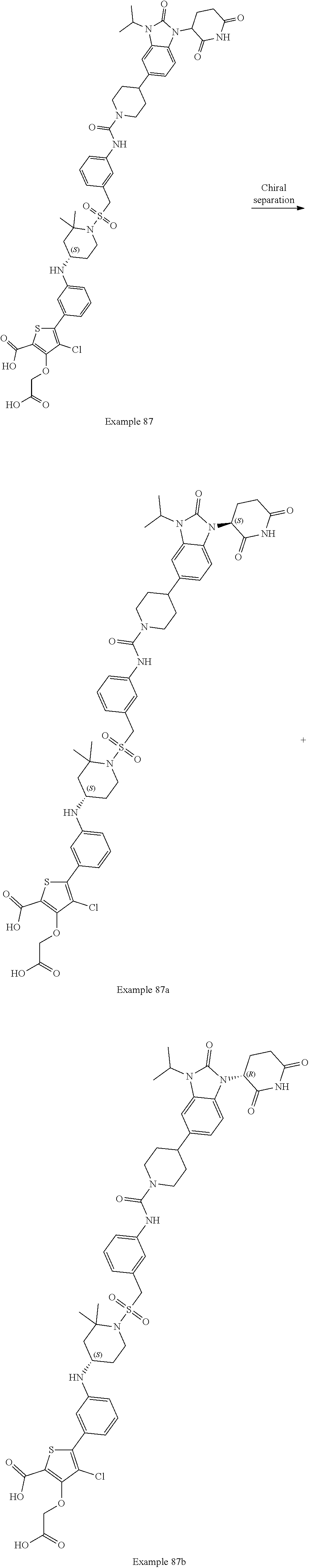

3-(carboxymethoxy)-4-chloro-5-[3-[[(4S)-1-[3-[4-[1-(2,6-dioxo-3-piperidyl)-3-isopropyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (87, 50 mg, 44.70 μmol, TFA salt) was subjected to chiral SFC purification to separate the diastereomers. Method details: Column Name: YMC Cellulose-SC, Flowrate: 5 ml/min, Co-Solvent: 50%, Co-Solvent Name: 0.2% TFA in IPA:ACN (1:1), Injected Volume: 15 μL, Temperature: 35° C., Outlet Pressure: 100 bar.

### Chiral SFC:

### Method:

Instrument: PIC 175
Column: YMC Cellulose SC (250×30) mm, 5 μm;
Mobile Phase: CO₂: 0.5% Trifluoroacetic acid in Isopropyl alcohol:Acetonitrile (1:1):(50:50)
Total Flow: 90 g/min
Back pressure: 120 bar
Wavelength: 210 nm
Cycle time: 40 min

50 mg of Compound 87 was dissolved in 2 mL of ACN and injected 700 μL/injection.

After SFC purification, the first eluted fraction at rt 7.9 min was concentrated under reduced pressure at 40° C. and purified by reverse-phase preparative column chromatography [Silicycle C18 column, Mobile phase A: 0.1% TFA in water and Mobile Phase B: MeCN] to afford 3-(carboxymethoxy)-4-chloro-5-[3-[[(4S)-1-[[3-[[4-[3-isopropyl-2-oxo-1-[(35)-2,6-dioxo-3-piperidyl]benzimidazol-5-yl]piperidine-1-carbonyl]amino]phenyl]methylsulfonyl]-2,2-dimethyl-4-piperidyl]amino]phenyl]thiophene-2-carboxylic acid (87a, 7 mg, 6.23 μmol, 14% yield, TFA salt) as an off-white solid.

### Fraction 1: 87a:

¹HNMR (400 MHz, DMSO-d6): δ 11.07 (s, 1H), 8.62 (s, 1H), 7.58 (s, 1H), 7.48 (d, J=8.00 Hz, 1H), 7.29-7.16 (m, 3H), 7.03-6.98 (m, 2H), 6.92 (d, J=7.60 Hz, 1H), 6.87 (s, 1H), 6.81 (d, J=7.60 Hz, 1H), 6.69 (dd, J=1.60, 8.40 Hz, 1H), 5.69 (bs, 1H), 5.39-5.28 (m, 1H), 4.90 (s, 2H), 4.63-4.60 (m, 1H), 4.38-4.23 (m, 3H), 3.16-3.10 (m, 1H), 2.92-2.70 (m, 3H), 2.69-2.64 (m, 2H), 2.02-1.95 (m, 1H), 1.89-1.72 (m, 4H), 1.66-1.58 (m, 2H), 1.49-1.40 (m, 11H), 1.18-1.07 (m, 1H).

LCMS (ES+): m/z 1004.2 [M+H]⁺

Similarly, the second eluted fraction at rt 21.3 min was concentrated under reduced pressure at 40° C. and purified by reverse-phase preparative column chromatography to afford 3-(carboxymethoxy)-4-chloro-5-[3-[(4S)-1-[3-[4-[3-isopropyl-2-oxo-1-[(3R)-2,6-dioxo-3-piperidyl]benzimidazol-5-yl]piperidine-1-carbonyl]amino]phenyl]methylsulfonyl]-2,2-dimethyl-4-piperidyl]amino]phenyl]thiophene-2-carboxylic acid (87b, 5 mg, 4.47 μmol, 10% yield, TFA salt) as an off-white solid.

### Fraction 2: 87b:

¹HNMR (400 MHz, DMSO-d6): δ 11.07 (s, 1H), 8.62 (s, 1H), 7.58 (s, 1H), 7.48 (d, J=8.00 Hz, 1H), 7.27-7.16 (m, 3H), 7.02-6.98 (m, 2H), 6.92 (d, J=7.60 Hz, 1H), 6.85 (s, 1H), 6.79 (d, J=7.60 Hz, 1H), 6.67 (dd, J=1.60, 8.20 Hz, 1H), 5.69 (s, 1H), 5.39-5.26 (m, 1H), 4.78 (s, 2H), 4.63-4.60 (m, 1H), 4.38-4.23 (m, 4H), 3.16-3.12 (m, 1H), 2.92-2.71 (m, 5H), 2.70-2.67 (m, 3H), 2.02-1.93 (m, 1H), 1.88-1.76 (m, 4H), 1.66-1.63 (m, 2H), 1.49-1.40 (m, 12H), 1.18-1.07 (m, 1H).

LCMS (ES+): m/z 1004.2 [M+H]⁺.

### Example 88

### 3-(carboxymethoxy)-4-chloro-5-(3-(((4S)-1-((3-(4-(1-(2,6-dioxopiperidin-3-yl)-3-ethyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (88)

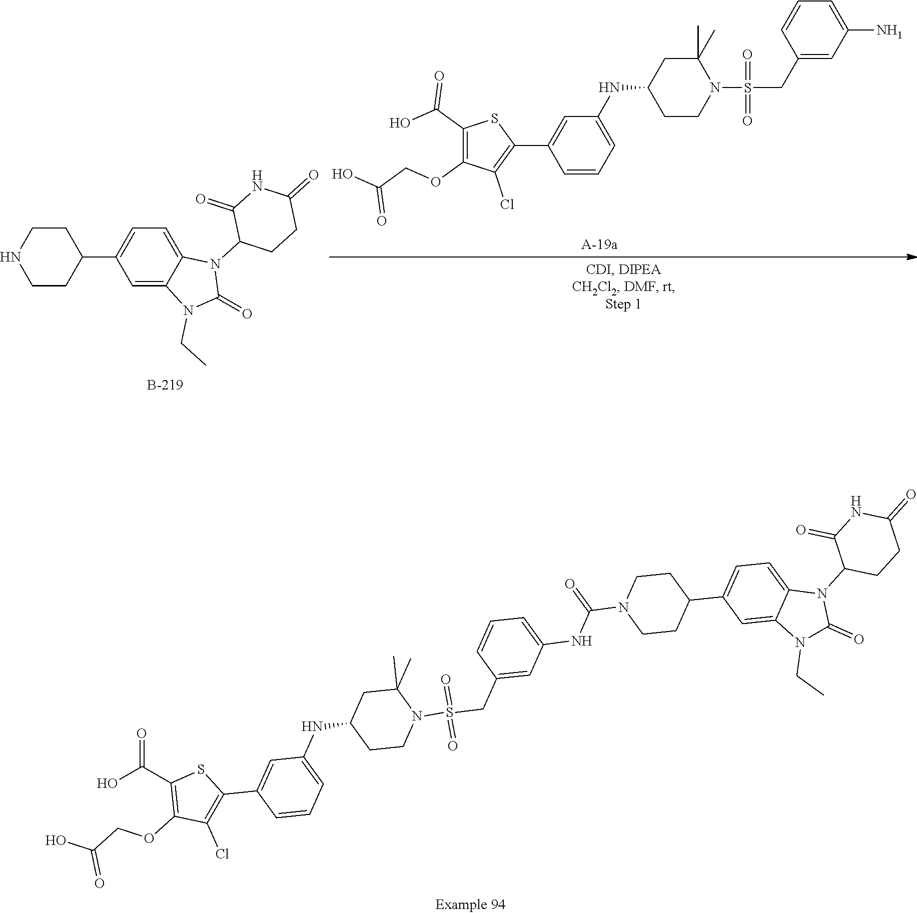

### Step 1: 3-(carboxymethoxy)-4-chloro-5-(3-(((4S)-1-((3-(4-(1-(2,6-dioxopiperidin-3-yl)-3-ethyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (88)

3-(carboxymethoxy)-4-chloro-5-(3-(((4S)-1-((3-(4-(1-(2,6-dioxopiperidin-3-yl)-3-ethyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (88, 9 mg, 8.13 μmol, 7% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-ethyl-2-oxo-5-(4-piperidyl)benzimidazol-1-yl]piperidine-2,6-dione (B-219) in a similar fashion to Example 23, except that excess DIPEA was used as base and DMF:DCM was used as the solvent. The residue was subjected to reverse phase prep HPLC (Column: XBRIDGE; Mobile phase A: 0.1% TFA in water; Mobile phase B: MeCN). LCMS (ES+): m/z 990.0. [M+H]⁺. ¹H NMR (400 MHz, DMSO-d₆) δ 11.09 (s, 1H), 8.62 (s, 1H), 7.57 (s, 1H), 7.46 (d, J=8.3 Hz, 1H), 7.27-7.13 (m, 3H), 7.03-6.95 (m, 2H), 6.92 (d, J=8.2 Hz, 1H), 6.87 (s, 1H), 6.81 (d, J=7.6 Hz, 1H), 6.69 (d, J=8.3 Hz, 1H), 5.33 (dd, J=12.8, 5.5 Hz, 1H), 4.90 (s, 2H), 4.41-4.21 (m, 5H), 3.91-3.83 (m, 2H), 3.17-3.07 (m, 2H), 2.94-2.83 (m, 3H), 2.81-2.58 (m, 4H), 2.05-1.96 (m, 1H), 1.80 (d, J=12.9 Hz, 4H), 1.71-1.56 (m, 3H), 1.48 (s, 3H), 1.39 (s, 3H), 1.20 (t, J=7.1 Hz, 3H), 1.16-1.04 (m, 2H).

### Example 89

### 3-(carboxymethoxy)-4-chloro-5-(3-(((4S)-1-((3-(3-(((2-(2,6-dioxopiperidin-3-yl)-1-oxoisoindolin-4-yl))methyl)carbamoyl) azetidine-1-carboxamido)benzyl)sulfonyl)-2,2-dimethylpiperidin-4-yl)amino)phenyl)thiophene-2-carboxylic acid (89)

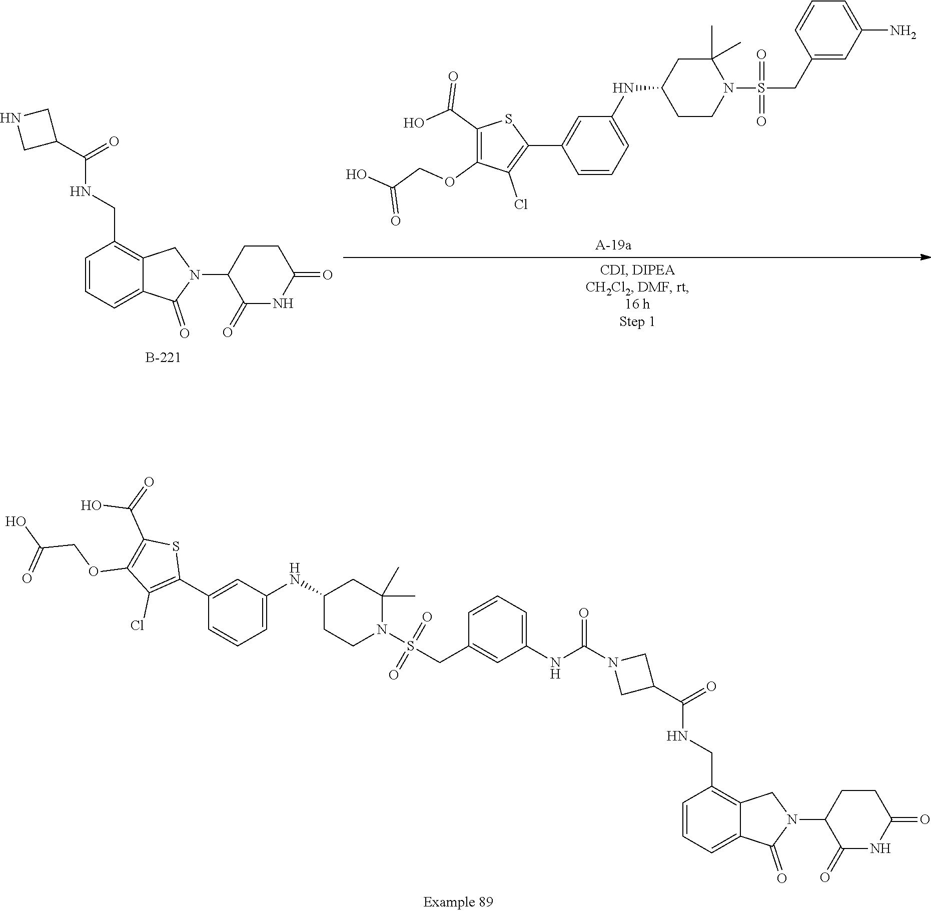 [SMILES: O=C1CCC(N2Cc3c(CNC(=O)C4CNC4)cccc3C2=O)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CC(C(=O)NCc3cccc4c3CN(C3CCC(=O)NC3=O)C4=O)C2)c1]

### Step 1: 3-(carboxymethoxy)-4-chloro-5-(3-(((4S)-1-((3-(3-(((2-(2,6-dioxopiperidin-3-yl)-1-oxoisoindolin-4-yl)methyl)carbamoyl) azetidine-1-carboxamido)benzyl)sulfonyl)-2,2-dimethylpiperidin-4-yl)amino)phenyl)thiophene-2-carboxylic acid (89)

3-(carboxymethoxy)-4-chloro-5-(3-(((4S)-1-((3-(3-(((2-(2,6-dioxopiperidin-3-yl)-1-oxoisoindolin-4-yl)methyl)carbamoyl) azetidine-1-carboxamido)benzyl)sulfonyl)-2,2-dimethylpiperidin-4-yl)amino)phenyl)thiophene-2-carboxylic acid (89, 8 mg, 7.18 μmol, 9% yield, formate salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and N-[[2-(2,6-dioxo-3-piperidyl)-1-oxo-isoindolin-4-yl]methyl]azetidine-3-carboxamide (B-221) in a similar fashion to Example 23, except excess DIPEA was used as base and DMF:DCM was used as the solvent. The residue was purified by reverse phase prep HPLC [Purification method: Column: XBridge, C18 (150×19 mm), 5 μm; Mobile phase A: 0.1% Formic acid in water and Mobile phase B: MeCN]. LCMS (ES+): m/z 990.2 [M+H]⁺. ¹H NMR (400 MHz, DMSO-d6): δ 11.02 (s, 1H), 8.61 (t, J=5.68 Hz, 1H), 8.57 (s, 1H), 7.65-7.63 (m, 1H), 7.56 (s, 1H), 7.53-7.48 (m, 3H), 7.26-7.16 (m, 2H), 6.98 (d, J=7.68 Hz, 1H), 6.84 (s, 1H), 6.79 (d, J=7.76 Hz, 1H), 6.67 (d, J=7.52 Hz, 1H), 5.65 (d, J=7.28 Hz, 1H), 5.16-5.12 (m, 1H), 4.79 (s, 2H), 4.51 (d, J=17.32 Hz, 1H), 4.40-4.34 (m, 4H), 4.24 (d, J=13.80 Hz, 1H), 4.11-3.99 (m, 4H), 3.37-3.09 (m, 1H), 2.95-2.89 (m, 2H), 2.70-2.60 (m, 2H), 2.50-2.40 (m, 1H), 2.03-1.99 (m, 1H), 1.87-1.76 (m, 2H), 1.46 (s, 3H), 1.41-1.39 (m, 4H).

### Example 90

### 3-(carboxymethoxy)-4-chloro-5-[3-[[(4S)-1-[3-[2-[1-[2-[[2-(2,6-dioxo-3-piperidyl)-1-oxo-isoindolin-4-yl]amino]acetyl]-4-piperidyl]oxy]acetyl]amino]phenyl]methylsulfonyl]-2,2-dimethyl-4-piperidyl]amino]phenyl]thiophene-2-carboxylic acid (90)

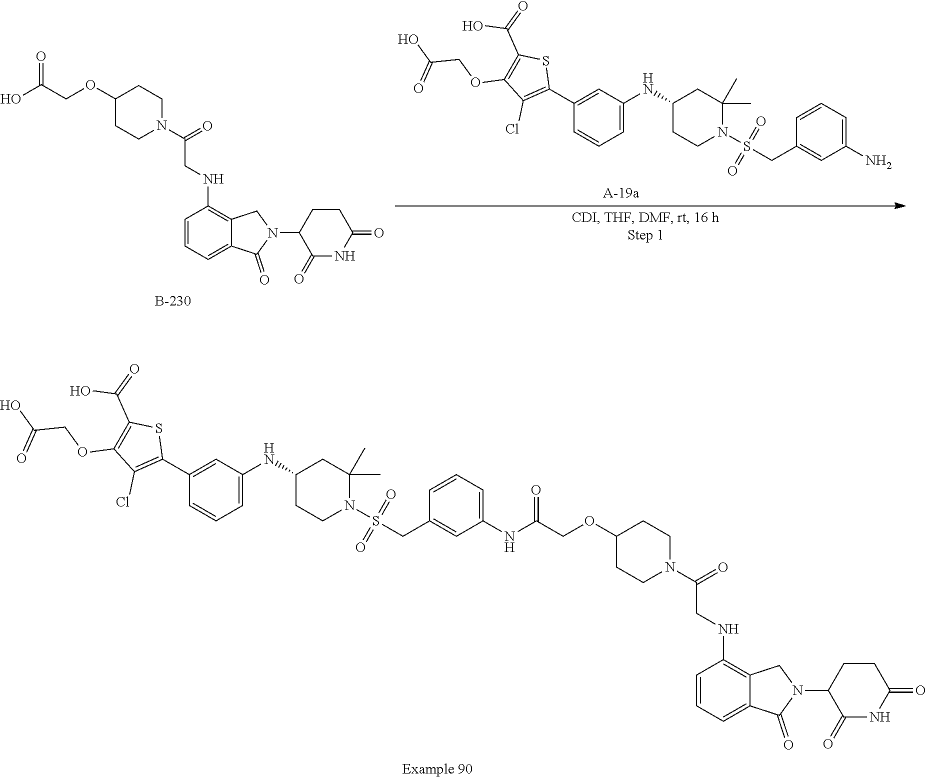 [SMILES: O=C(O)COC1CCN(C(=O)CNc2cccc3c2CN(C2CCC(=O)NC2=O)C3=O)CC1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)COC2CCN(C(=O)CNc3cccc4c3CN(C3CCC(=O)NC3=O)C4=O)CC2)c1]

### Step 1: 3-(carboxymethoxy)-4-chloro-5-[3-[[(4S)-1-[[3-[[2-[1-[2-[[2-(2,6-dioxo-3-piperidyl)-1-oxo-isoindolin-4-yl]amino]acetyl]-4-piperidyl]oxy]acetyl]amino]phenyl]methylsulfonyl]-2,2-dimethyl-4-piperidyl]amino]phenyl]thiophene-2-carboxylic acid (90)

3-(carboxymethoxy)-4-chloro-5-[3-[[(4S)-1-[3-[2-[[1-[2-[[2-(2,6-dioxo-3-piperidyl)-1-oxo-isoindolin-4-yl]amino]acetyl]-4-piperidyl]oxy]acetyl]amino]phenyl]methylsulfonyl]-2,2-dimethyl-4-piperidyl]amino]phenyl]thiophene-2-carboxylic acid (90, 22.5 mg, 19.10 μmol, 23% yield, TFA salt) was synthesized from 2-[[1-[2-[[2-(2,6-dioxo-3-piperidyl)-1-oxo-isoindolin-4-yl]amino]acetyl]-4-piperidyl]oxy]acetic acid (B-224) and of 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except THF:DMF was used as the solvent. The residue was purified by reverse phase prep HPLC [Purification method: Column: XBRIDGE C8 (19×150 mm), 5 μm; Mobile phase A: 0.1% TFA in water and Mobile phase B: MeCN]. LCMS (ES+): m/z 1048.1 [M+H]⁺. ¹H NMR (400 MHz, DMSO-d6): δ 11.05 (s, 1H), 9.75 (s, 1H), 7.76 (s, 1H), 7.62 (d, J=8.40 Hz, 1H), 7.35-7.27 (m, 2H), 7.23-7.19 (m, 1H), 7.14-7.10 (m, 1H), 7.00-6.97 (m, 1H), 6.88 (s, 1H), 6.82-6.76 (m, 2H), 6.71 (d, J=8.00 Hz, 1H), 5.13-5.09 (m, 1H), 4.92 (s, 2H), 4.42 (d, J=14.00 Hz, 1H), 4.33-4.28 (m, 2H), 4.20-4.14 (m, 3H), 4.05 (s, 2H), 3.92-3.66 (m, 3H), 3.14-3.09 (m, 2H), 2.97-2.88 (m, 1H), 2.68-2.63 (m, 1H), 2.41-2.30 (m, 1H), 2.04-1.77 (m, 5H), 1.65-1.52 (m, 1H), 1.46-1.39 (m, 8H), 1.20-1.00 (m, 1H).

### Example 91

### 3-(carboxymethoxy)-4-chloro-5-(3-(((4S)-1-((3-(3-(3-(1-(2,6-dioxopiperidin-3-yl)-3-methyl-2-oxo-2,3-dihydro-1H-benzo[d]imidazol-5-yl)prop-2-yn-1-yl)ureido)benzyl)sulfonyl)-2,2-dimethylpiperidin-4-yl)amino)phenyl)thiophene-2-carboxylic acid (91)

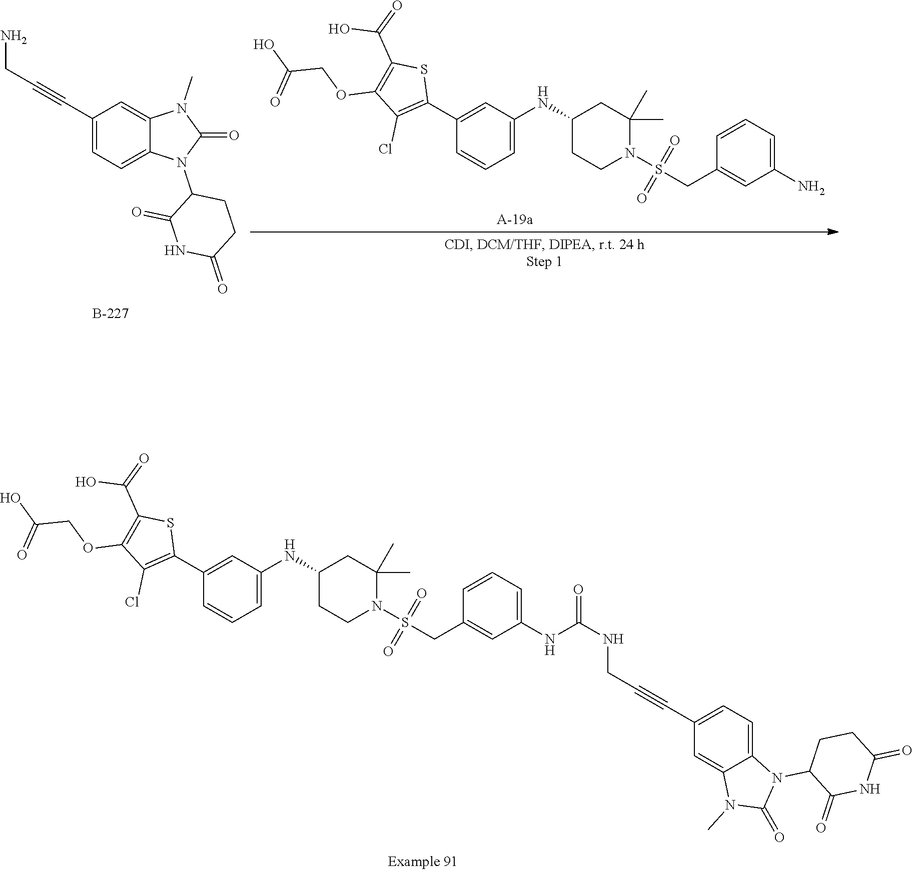 [SMILES: Cn1c(=O)n(C2CCC(=O)NC2=O)c2ccc(C#CCN)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(C#CCNC(=O)Nc3cccc(CS(=O)(=O)N4CC[C@H](Nc5cccc(-c6sc(C(=O)O)c(OCC(=O)O)c6Cl)c5)CC4(C)C)c3)cc21]

### Step 1: 3-(carboxymethoxy)-4-chloro-5-(3-(((4S)-1-((3-(3-(3-(1-(2,6-dioxopiperidin-3-yl)-3-methyl-2-oxo-2,3-dihydro-1H-benzo[d]imidazol-S-yl)prop-2-yn-1-yl)ureido)benzyl)sulfonyl)-2,2-dimethylpiperidin-4-yl)amino)phenyl)thiophene-2-carboxylic acid (91)

3-(carboxymethoxy)-4-chloro-5-(3-(((4S)-1-((3-(3-(3-(1-(2,6-dioxopiperidin-3-yl)-3-methyl-2-oxo-2,3-dihydro-1H-benzo[d]imidazol-5-yl)prop-2-yn-1-yl)ureido)benzyl)sulfonyl)-2,2-dimethylpiperidin-4-yl)amino)phenyl)thiophene-2-carboxylic acid (91, 5.35 mg, 5.02 μmol, 6% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(3-aminoprop-1-ynyl)-3-methyl-2-oxo-benzimidazol-1-yl]piperidine-2,6-dione (B-227) in a similar fashion to Example 23, except excess DIPEA was used as base and DCM:DMF was used as the solvent. The residue was purified by reverse phase prep HPLC [Purification method: Column: XSelect C18 (150×19 mm) 5 μm, Mobile phase-A: 0.1% TFA in water, Mobile phase-B: MeCN]. LCMS (ES+): m/z 946.0 [M+H]⁺. ¹H NMR (400 MHz, DMSO-d₆) δ 11.11 (s, 1H), 8.73 (s, 1H), 7.48 (s, 1H), 7.42 (d, J=8.2 Hz, 1H), 7.29-7.16 (m, 3H), 7.15-7.08 (m, 2H), 6.99-6.94 (m, 1H), 6.88 (s, 1H), 6.80 (d, J=7.5 Hz, 1H), 6.70 (d, J=8.1 Hz, 1H), 6.60-6.55 (m, 1H), 5.37 (dd, J=12.8, 5.4 Hz, 1H), 4.91 (s, 2H), 4.37 (d, J=13.8 Hz, 1H), 4.25 (d, J=13.7 Hz, 1H), 4.17 (d, J=5.4 Hz, 2H), 2.87 (t, J=14.3 Hz, 2H), 2.76-2.69 (m, 1H), 2.65-2.58 (m, 2H), 2.07-1.98 (m, 2H), 1.92-1.83 (m, 1H), 1.77 (d, J=13.1 Hz, 1H), 1.45 (s, 3H), 1.42-1.34 (m, 4H), 1.20-1.06 (m, 1H).

### Example 92

### 3-(carboxymethoxy)-4-chloro-5-[3-[[(4S)-1-[3-[2-[1-[2-[[2-(2,6-dioxo-3-piperidyl)-1-oxo-isoindolin-4-yl]amino]acetyl]azetidin-3-yl]oxyacetyl]amino]phenyl]methylsulfonyl]-2,2-dimethyl-4-piperidyl]amino]phenyl]thiophene-2-carboxylic acid (92)

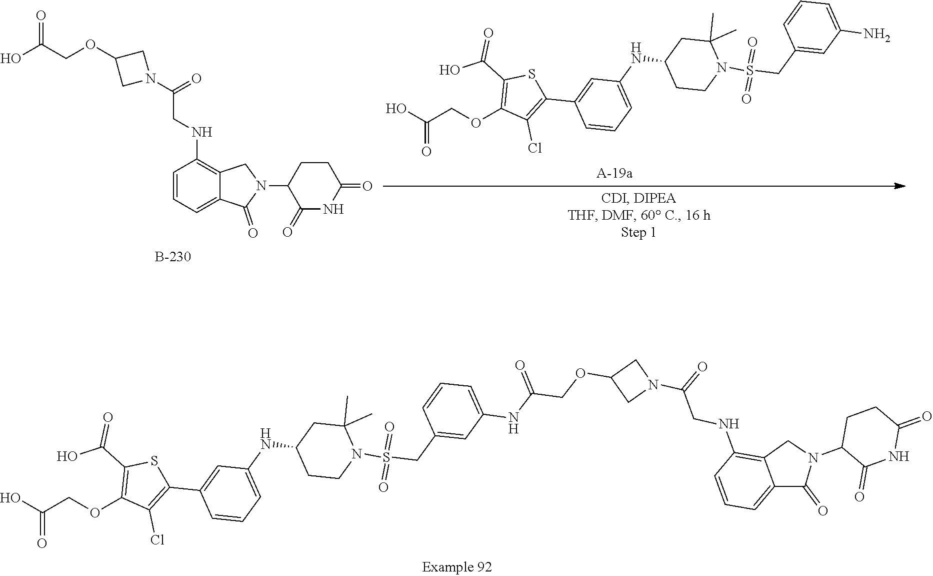 [SMILES: O=C(O)COC1CN(C(=O)CNc2cccc3c2CN(C2CCC(=O)NC2=O)C3=O)C1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)COC2CN(C(=O)CNc3cccc4c3CN(C3CCC(=O)NC3=O)C4=O)C2)c1]

### Step 1: 3-(carboxymethoxy)-4-chloro-5-[3-[[(4S)-1-[3-[2-[1-[2-[[2-(2,6-dioxo-3-piperidyl)-1-oxo-isoindolin-4-yl]amino]acetyl]azetidin-3-yl]oxyacetyl]amino]phenyl]methylsulfonyl]-2,2-dimethyl-4-piperidyl]amino]phenyl]thiophene-2-carboxylic acid (92)

3-(carboxymethoxy)-4-chloro-5-[3-[[(4S)-1-[[3-[2-[1-[2-[[2-(2,6-dioxo-3-piperidyl)-1-oxo-isoindolin-4-yl]amino]acetyl]azetidin-3-yl]oxyacetyl]amino]phenyl]methylsulfonyl]-2,2-dimethyl-4-piperidyl]amino]phenyl]thiophene-2-carboxylic acid (92, 5 mg, 4.09 μmol, 6% yield) was synthesized from 2-[1-[2-[[2-(2,6-dioxo-3-piperidyl)-1-oxo-isoindolin-4-yl]amino]acetyl]azetidin-3-yl]oxyacetic acid (B-230) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except excess DIPEA was used as base, THF:DMF was used as the solvent and the reaction was heated to 60° C. The residue was purified by reverse phase prep-HPLC [Purification method: Column: X-Bridge C18 (150×19) mm 5 μm; Mobile phase A: 0.1% TFA in water; Mobile phase B: acetonitrile]. LCMS (ES+): m/z 1020.0 [M+H]⁺. ¹H NMR (400 MHz, DMSO-d₆) δ 11.01 (s, 1H), 9.91 (s, 1H), 7.72 (s, 1H), 7.64-7.59 (m, 1H), 7.33 (t, J=7.9 Hz, 1H), 7.28 (t, J=7.7 Hz, 1H), 7.23-7.17 (m, 2H), 7.12 (d, J=7.6 Hz, 1H), 7.00-6.95 (m, 1H), 6.89-6.85 (m, 1H), 6.82-6.78 (m, 1H), 6.69 (dd, J=8.4, 2.4 Hz, 1H), 6.66 (d, J=8.1 Hz, 1H), 5.67 (s, 1H), 5.11 (dd, J=13.3, 5.1 Hz, 1H), 4.91 (s, 2H), 4.52-4.38 (m, 3H), 4.32-4.24 (m, 2H), 4.21-4.16 (m, 2H), 4.14-4.07 (m, 2H), 3.86 (d, J=3.6 Hz, 1H), 3.83 (s, 2H), 3.16-3.07 (m, 2H), 2.99-2.85 (m, 2H), 2.66-2.57 (m, 1H), 2.42-2.35 (m, 1H), 2.07-1.98 (m, 1H), 1.92-1.83 (m, 1H), 1.82-1.74 (m, 1H), 1.45 (s, 3H), 1.43-1.34 (m, 4H), 1.17-1.03 (m, 1H).

### Example 93

### 3-(carboxymethoxy)-4-chloro-5-(3-((1-((3-(1-(2-((2-(2,6-dioxopiperidin-3-yl)-1-oxoisoindolin-4-yloxy) acetyl) azetidine-3-carboxamido)-4-fluorobenzyl)sulfonyl)piperidin-4-yl)amino)phenyl)thiophene-2-carboxylic acid (93)

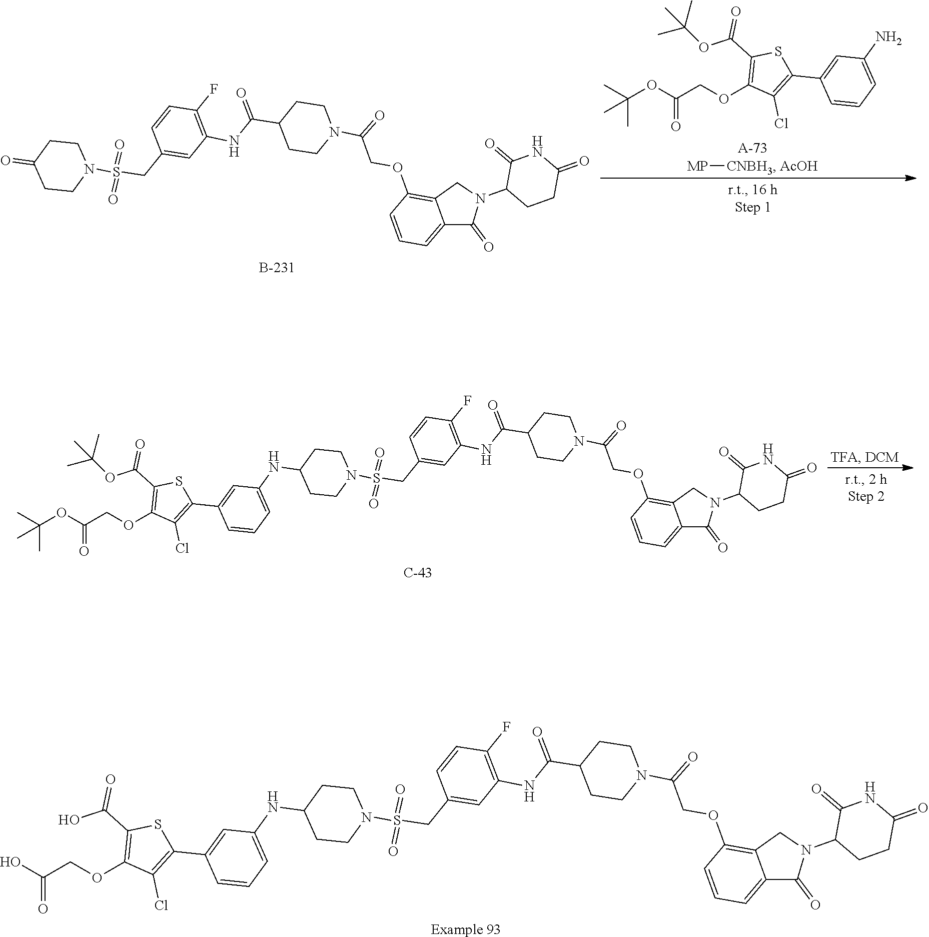

### Step 1: tert-butyl 3-(2-tert-butoxy-2-oxo-ethoxy)-4-chloro-5-[3-[[1-[[3-[[1-[2-[[2-(2,6-dioxo-3-piperidyl)-1-oxo-isoindolin-4-yl]oxyacetyl]azetidine-3-carbonyl]amino]-4-fluoro-phenyl]methylsulfonyl]-4-piperidyl]amino]phenyl]thiophene-2-carboxylate (C-43)

tert-butyl 3-(2-tert-butoxy-2-oxo-ethoxy)-4-chloro-5-[3-[[1-[[3-[1-[2-[[2-(2,6-dioxo-3-piperidyl)-1-oxo-isoindolin-4-yl]oxyacetyl]azetidine-3-carbonyl]amino]-4-fluoro-phenyl]methylsulfonyl]-4-piperidyl]amino]phenyl]thiophene-2-carboxy late (C-43, 150 mg, 50.39 μmol, 42% yield, 37% purity) was synthesized from 1-[2-[[2-(2,6-dioxo-3-piperidyl)-1-oxo-isoindolin-4-yl]oxyacetyl]-N-[2-fluoro-5-[(4-oxo-1-piperidyl)sulfonylmethyl]phenyl]azetidine-3-carboxamide (B-231) and tert-butyl 5-(3-aminophenyl)-3-(2-tert-butoxy-2-oxo-ethoxy)-4-chloro-thiophene-2-carboxylate (A-73) in a similar fashion to Compound C-38. LCMS (ES−): m/z 1091.3 [M−H]⁻.

### Step 2: 3-(carboxymethoxy)-4-chloro-5-(3-((1-((3-(1-(2-((2-(2,6-dioxopiperidin-3-yl)-1-oxoisoindolin-4-yl)oxy) acetyl) azetidine-3-carboxamido)-4-fluorobenzyl)sulfonyl)piperidin-4-yl)amino)phenyl)thiophene-2-carboxylic acid (93)

3-(carboxymethoxy)-4-chloro-5-(3-((1-((3-(1-(2-((2-(2,6-dioxopiperidin-3-yl)-1-oxoisoindolin-4-yl)oxy) acetyl) azetidine-3-carboxamido)-4-fluorobenzyl)sulfonyl)piperidin-4-yl)amino)phenyl)thiophene-2-carboxylic acid (93, 15 mg, 13.23 μmol, 27% yield, TFA salt) was synthesized from tert-butyl 3-(2-tert-butoxy-2-oxo-ethoxy)-4-chloro-5-[3-[[1-[[3-[[1]-[2-[[2-(2,6-dioxo-3-piperidyl)-1-oxo-isoindolin-4-yl]oxyacetyl]azetidine-3-carbonyl]amino]-4-fluoro-phenyl]methylsulfonyl]-4-piperidyl]amino]phenyl]thiophene-2-carboxylate (C-43) in a similar fashion to Compound C-1, except using 78 eq. of TFA. The residue was subjected to reverse phase prep HPLC [Purification method: column: XBRIDGE C8 (19×150 mm) 5 μm, mobile phase A: 0.1% TFA in water, mobile phase B: MeCN]. LCMS (ES+): m/z 981.0 [M+H]⁺. ¹H NMR (400 MHz, DMSO-d₆) δ 10.98 (s, 1H), 10.00 (s, 1H), 8.00 (dd, J=7.5, 2.1 Hz, 1H), 7.47 (t, J=7.8 Hz, 1H), 7.34 (d, J=7.5 Hz, 1H), 7.32-7.26 (m, 1H), 7.24-7.13 (m, 3H), 6.89-6.86 (m, 1H), 6.81 (d, J=7.6 Hz, 1H), 6.71 (dd, J=8.3, 2.2 Hz, 1H), 5.09 (dd, J=13.2, 5.1 Hz, 1H), 4.92 (s, 2H), 4.79 (s, 2H), 4.51-4.42 (m, 1H), 4.42-4.37 (m, 3H), 4.27 (d, J=17.4 Hz, 1H), 4.15-4.01 (m, 2H), 3.79-3.71 (m, 2H), 3.02-2.85 (m, 4H), 2.62-2.54 (m, 1H), 2.04-1.89 (m, 3H), 1.43-1.31 (m, 2H).

### Example 94

### 3-(carboxymethoxy)-4-chloro-5-[3-[[(4S)-1-[[3-[[4-[1-(2,6-dioxo-3-piperidyl)indolin-5-yl]piperidine-1-carbonyl]amino]phenyl]methylsulfonyl]-2,2-dimethyl-4-piperidyl]amino]phenyl]thiophene-2-carboxylic acid (94)

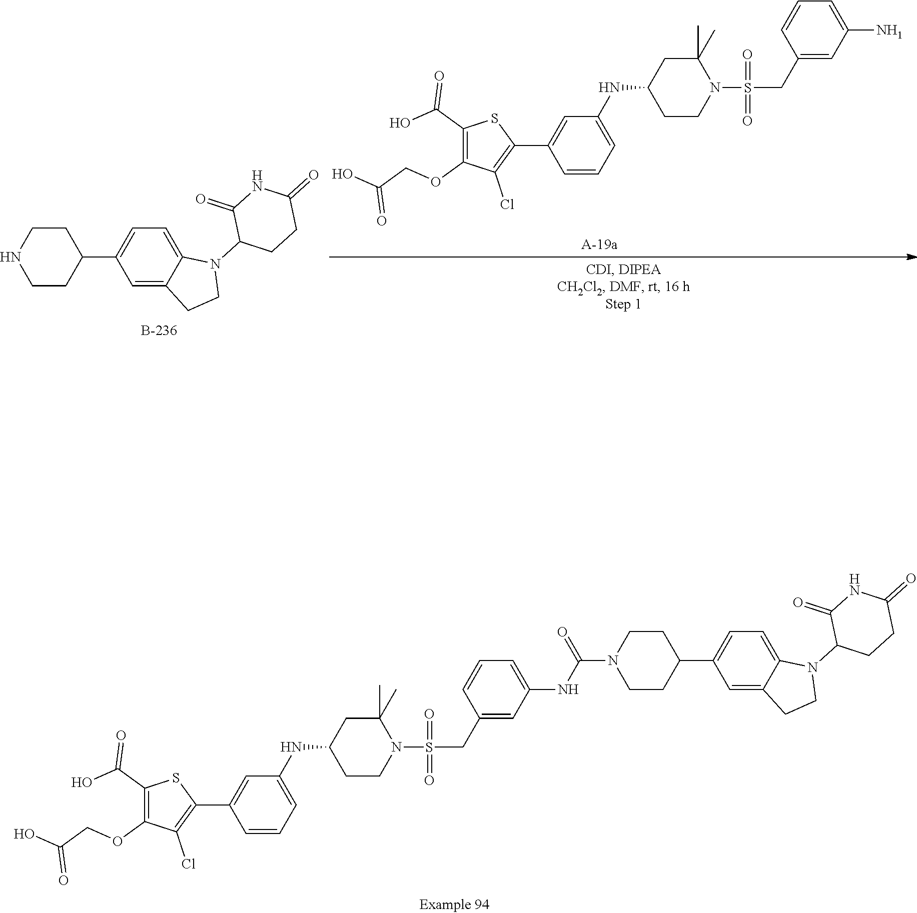

### Step 1: 3-(carboxymethoxy)-4-chloro-5-[3-[[(4S)-1-[[3-[[4-[1-(2,6-dioxo-3-piperidyl)indolin-5-yl]piperidine-1-carbonyl]amino]phenyl]methylsulfonyl]-2,2-dimethyl-4-piperidyl]amino]phenyl]thiophene-2-carboxylic acid (94)

3-(carboxymethoxy)-4-chloro-5-[3-[[(4S)-1-[[3-[[4-[1-(2,6-dioxo-3-piperidyl)indolin-5-yl]piperidine-1-carbonyl]amino]phenyl]methylsulfonyl]-2,2-dimethyl-4-piperidyl]amino]phenyl]thiophene-2-carboxylic acid (94, 6 mg, 4.60 μmol, 5% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(4-piperidyl)indolin-1-yl]piperidine-2,6-dione (B-236) in a similar fashion to Example 23, except using excess DIPEA as base and DCM:DMF as the solvent. The residue was purified by reverse phase prep HPLC (Column: XSelect C18 (150×19 mm) 5 μm, Mobile phase: 0.1% TFA in MQ water/MeCN). LCMS (ES+): m/z 947.2 [M+H]⁺. ¹H NMR (400 MHz, DMSO-d₆) δ 10.78 (s, 1H), 8.63-8.56 (m, 1H), 7.56 (s, 1H), 7.47-7.42 (m, 1H), 7.26-7.16 (m, 2H), 7.01-6.95 (m, 1H), 6.91 (s, 1H), 6.88-6.84 (m, 1H), 6.81 (d, J=7.7 Hz, 2H), 6.72-6.65 (m, 1H), 6.39 (d, J=8.2 Hz, 1H), 4.90 (s, 2H), 4.57 (dd, J=13.0, 4.9 Hz, 1H), 4.35 (d, J=13.8 Hz, 1H), 4.30-4.21 (m, 3H), 3.18-3.07 (m, 3H), 2.92-2.73 (m, 6H), 2.61-2.54 (m, 2H), 2.24-2.11 (m, 1H), 1.95-1.70 (m, 5H), 1.59-1.42 (m, 6H), 1.38 (s, 3H), 1.16-1.03 (m, 2H).

### Example 95

### 3-(carboxymethoxy)-4-chloro-5-[3-[[(4S)-1-[[3-[[4-[1-(2,6-dioxo-3-piperidyl)-2-methyl-indolin-5-yl]piperidine-1-carbonyl]amino]phenyl]methylsulfonyl]-2,2-dimethyl-4-piperidyl]amino]phenyl]thiophene-2-carboxylic acid (95)

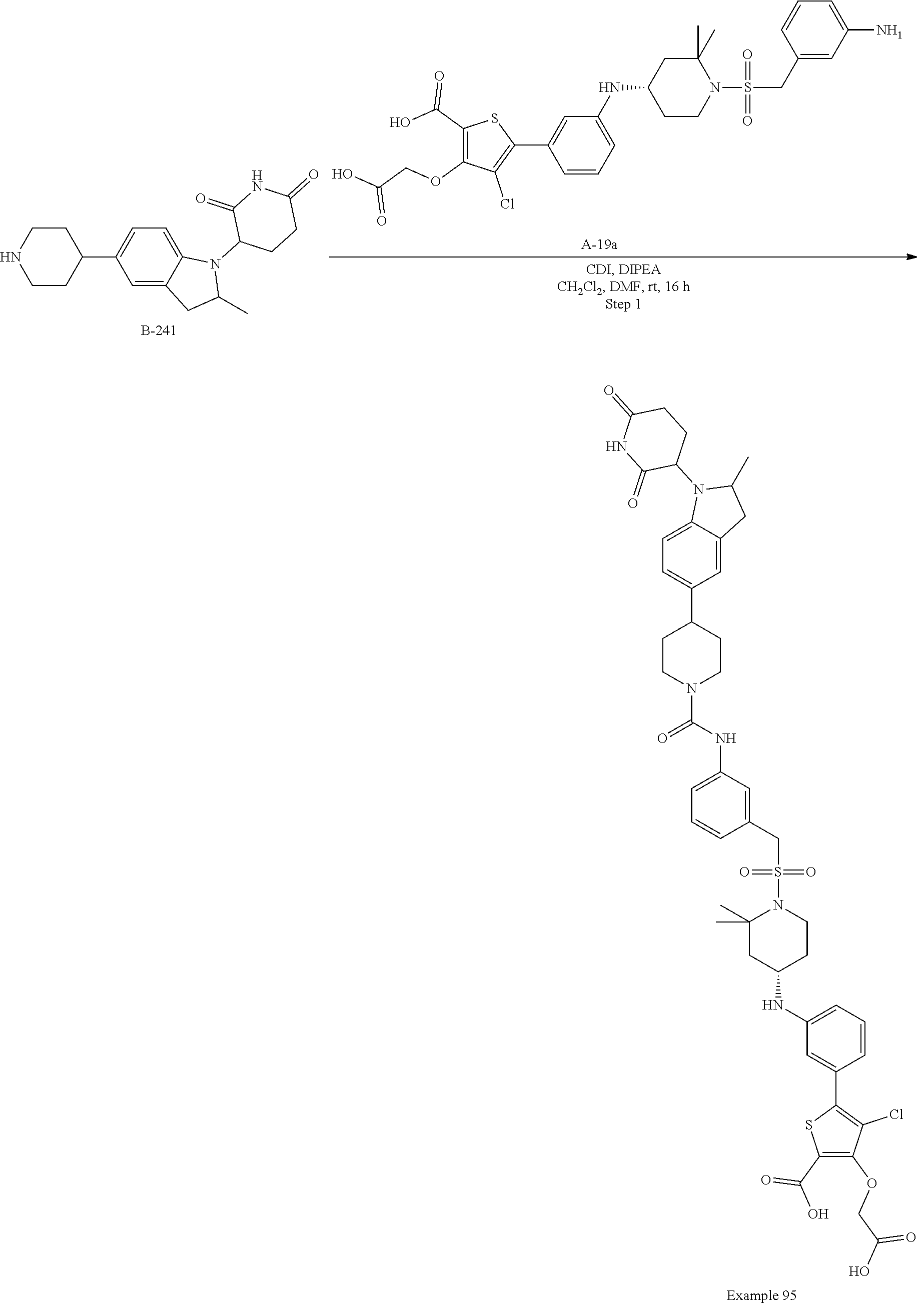

### Step 1: 3-(carboxymethoxy)-4-chloro-5-[3-[[(4S)-1-[[3-[[4-[1-(2,6-dioxo-3-piperidyl)-2-methyl-indolin-5-yl]piperidine-1-carbonyl]amino]phenyl]methylsulfonyl]-2,2-dimethyl-4-piperidyl]amino]phenyl]thiophene-2-carboxylic acid (95)

3-(carboxymethoxy)-4-chloro-5-[3-[[(4S)-1-[[3-[[4-[1-(2,6-dioxo-3-piperidyl)-2-methyl-indolin-5-yl]piperidine-1-carbonyl]amino]phenyl]methylsulfonyl]-2,2-dimethyl-4-piperidyl]amino]phenyl]thiophene-2-carboxylic acid (95, 3.96 mg, 3.21 μmol, 5% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[2-methyl-5-(4-piperidyl)indolin-1-yl]piperidine-2,6-dione (B-241) in a similar fashion to Example 23, except using excess DIPEA as base and DMF as the solvent. The residue was purified by reverse phase preparative HPLC [Purification method: Column: XSELECT C18 (150×19 mm), 5 μm; Mobile phase A-0.1% TFA in water and Mobile phase B: MeCN]. LCMS (ES+): m/z 961.2 [M+H]⁺. ¹H NMR (400 MHz, DMSO-d6): 10.78 (s, 1H), 8.59 (s, 1H), 7.56 (s, 1H), 7.45 (d, J=8.04 Hz, 1H), 7.26-7.14 (m, 3H), 6.98 (d, J=7.88 Hz, 1H), 6.87-6.80 (m, 2H), 6.81 (d, J=6.92 Hz, 1H), 6.69 (d, J=7.80 Hz, 2H), 4.91 (s, 2H), 4.37-4.24 (m, 5H), 3.14-3.05 (m, 3H), 2.87-2.80 (m, 1H), 1.90-1.71 (m, 6H), 1.52-1.32 (m, 14H), 1.22 (d, J=6.00 Hz, 3H), 1.15-1.05 (m, 2H).

### Example 96

### 3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]azetidin-3-yl]oxycarbonylamino]phenyl]methylsulfonyl]-2,2-dimethyl-4-piperidyl]amino]phenyl]thiophene-2-carboxylic acid (96)

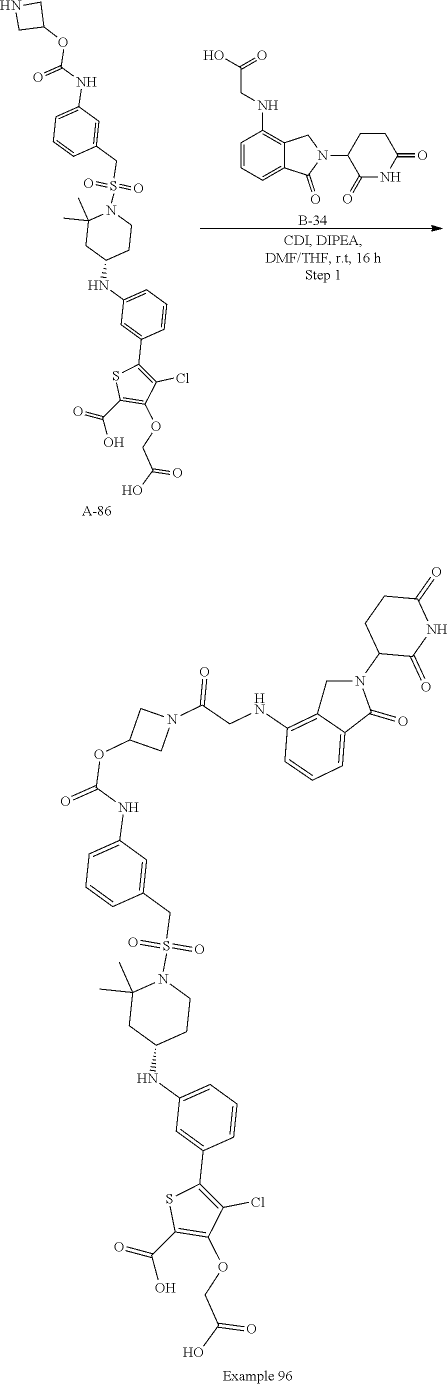

### Step 1: 3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]azetidin-3-yl]oxycarbonylamino]phenyl]methylsulfonyl]-2,2-dimethyl-4-piperidyl]amino]phenyl]thiophene-2-carboxylic acid (96)

3-(carboxymethoxy)-4-chloro-5-[3-[[(4S)-1-[3-[[1-[2-[[2-(2,6-dioxo-3-piperidyl)-1-oxo-isoindolin-4-yl]amino]acetyl]azetidin-3-yl]oxycarbonylamino]phenyl]methylsulfonyl]-2,2-dimethyl-4-piperidyl]amino]phenyl]thiophene-2-carboxylic acid (96, 7 mg, 6.07 μmol, 33% yield, TFA salt) was synthesized from 5-[3-[[(4S)-1-[3-(azetidin-3-yloxycarbonylamino)phenyl]methylsulfonyl]-2,2-dimethyl-4-piperidyl]amino]phenyl]-3-(carboxymethoxy)-4-chloro-thiophene-2-carboxylic acid (A-86) and 2-[[2-(2,6-dioxo-3-piperidyl)-1-oxo-isoindolin-4-yl]amino]acetic acid (B-34) in a similar fashion to Example 23, except using excess DIPEA as base and THF:DMF as the solvent. The residue was purified by reverse phase column chromatography [Purification method: Column: X bridge C18 (150×19) mm, 5 μm; Mobile phase A: 0.1% TFA in MQ-water; Mobile phase B: Acetonitrile. LCMS (ES+): m/z 1006.0 [M+H]⁺. ¹H NMR (400 MHz, DMSO-d₆) δ 11.01 (s, 1H), 10.01 (s, 1H), 7.57 (s, 1H), 7.42 (d, J=8.2 Hz, 1H), 7.29 (td, J=7.8, 3.0 Hz, 2H), 7.19 (t, J=7.9 Hz, 1H), 7.06 (d, J=7.7 Hz, 1H), 6.98 (d, J=7.4 Hz, 1H), 6.89-6.85 (m, 1H), 6.83-6.78 (m, 1H), 6.73-6.65 (m, 2H), 5.26-5.18 (m, 1H), 5.11 (dd, J=13.3, 5.1 Hz, 1H), 4.91 (s, 2H), 4.61 (t, J=8.7 Hz, 1H), 4.39 (d, J=13.8 Hz, 1H), 4.32-4.20 (m, 4H), 4.16 (d, J=17.1 Hz, 1H), 3.93-3.83 (m, 3H), 3.16-3.07 (m, 2H), 2.98-2.85 (m, 1H), 2.65-2.57 (m, 1H), 2.40-2.27 (m, 1H), 2.07-1.98 (m, 1H), 1.93-1.84 (m, 1H), 1.78 (d, J=12.7 Hz, 1H), 1.45 (s, 3H), 1.42-1.33 (m, 4H), 1.17-1.03 (m, 1H).

### Example 97

### 5-[3-[[(4S)-1-[[3-[[(3aR,6aS)-2-[2-[[2-(2,6-dioxo-3-piperidyl)-1-oxo-isoindolin-4-yl]amino]acetyl]-3,3a,4,5,6,6a-hexahydro-1H-cyclopenta[c]pyrrole-S-carbonyl]amino]phenyl]methylsulfonyl]-2,2-dimethyl-4-piperidyl]amino]phenyl]-3-(carboxymethoxy)-4-chloro-thiophene-2-carboxylic acid (97)

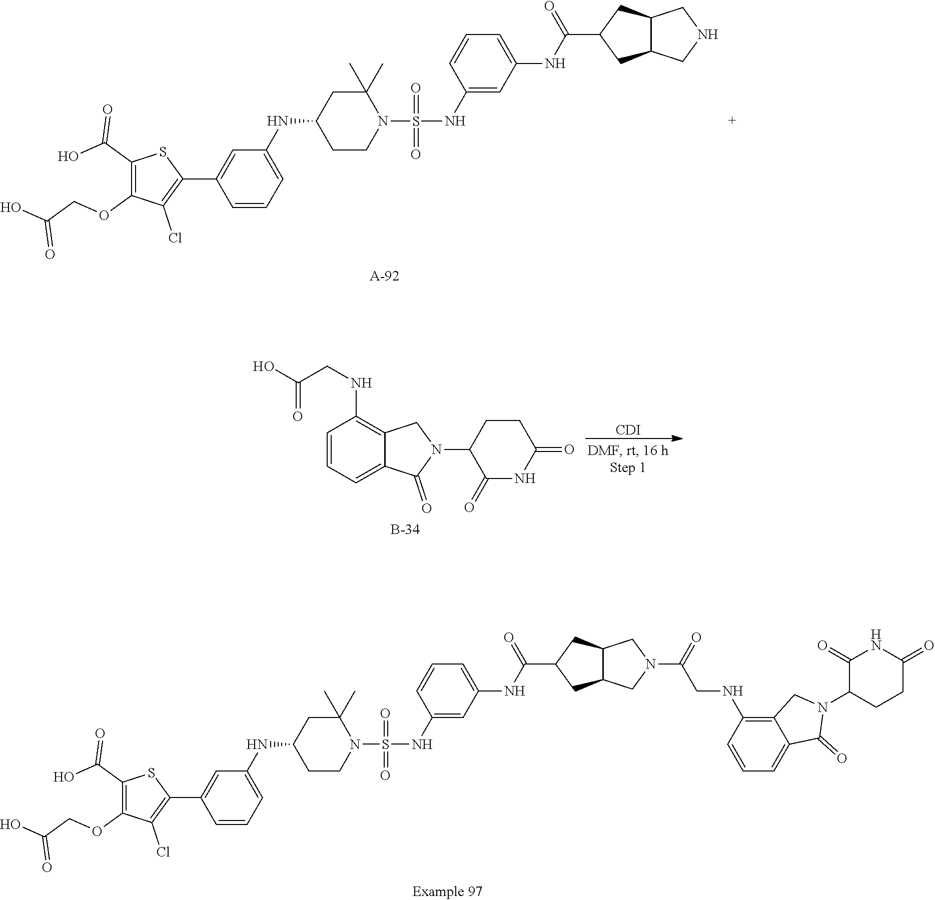

### Step 1: 5-[3-[[(4S)-1-[[3-[(3aR,6aS)-2-[2-[[2-(2,6-dioxo-3-piperidyl)-1-oxo-isoindolin-4-yl]amino]acetyl]-3,3a,4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (97)

5-[3-[[(4S)-1-[[3-[[(3aR,6aS)-2-[2-[[2-(2,6-dioxo-3-piperidyl)-1-oxo-isoindolin-4-yl]amino]acetyl]-3,3a,4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (97, 9 mg, 6.5 μmol, 10% yield, TFA salt) was synthesized from 2-[[2-(2,6-dioxo-3-piperidyl)-1-oxo-isoindolin-4-yl]amino]acetic acid (B-34) and 5-[3-[[(4S)-1-[[3-[[(3aR,6aS)-1,2,3,3a,4,5,6,6a-octahydrocyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-92) in a similar fashion to Example 23, except using DMF as the solvent. The residue was purified by reverse phase preparative HPLC [Purification method: Column: XSelect C18, 19×150 mm, 5 μm, Mobile phase A: 0.1% TFA in water and Mobile phase B: MeCN]. LCMS (ES+): m/z 1044.2 [M+H]⁺. ¹H NMR (400 MHz, DMSO-d₆) δ 11.00 (s, 1H), 9.98 (s, 1H), 7.69-7.65 (m, 1H), 7.61 (d, J=8.2 Hz, 1H), 7.33-7.25 (m, 2H), 7.20 (t, J=7.8 Hz, 1H), 7.07 (d, J=7.6 Hz, 1H), 6.97 (d, J=7.5 Hz, 1H), 6.88-6.85 (m, 1H), 6.83-6.79 (m, 1H), 6.75-6.66 (m, 2H), 5.11 (dd, J=13.3, 5.1 Hz, 1H), 4.91 (s, 2H), 4.38 (d, J=13.7 Hz, 1H), 4.30 (d, J=11.1 Hz, 1H), 4.27 (d, J=7.9 Hz, 1H), 4.17 (d, J=17.0 Hz, 1H), 3.95 (s, 2H), 3.70-3.62 (m, 3H), 3.15-3.07 (m, 2H), 3.03-2.87 (m, 3H), 2.81-2.71 (m, 1H), 2.66-2.57 (m, 2H), 2.43-2.34 (m, 1H), 2.21-2.09 (m, 2H), 2.06-1.96 (m, 1H), 1.90-1.83 (m, 1H), 1.78 (d, J=12.6 Hz, 1H), 1.73-1.55 (m, 2H), 1.46 (s, 3H), 1.44-1.35 (m, 4H), 1.16-1.02 (m, 1H).

### Example 98

### 3-(carboxymethoxy)-4-chloro-5-[3-[[(4S)-1-[[3-[(1S,5R)-3-[2-[[2-(2,6-dioxo-3-piperidyl)-1-oxo-isoindolin-4-yl]amino]acetyl]-3-azabicyclo[3.1.0]hexane-6-carbonyl]amino]phenyl]methylsulfonyl]-2,2-dimethyl-4-piperidyl]amino]phenyl]thiophene-2-carboxylic acid (98)

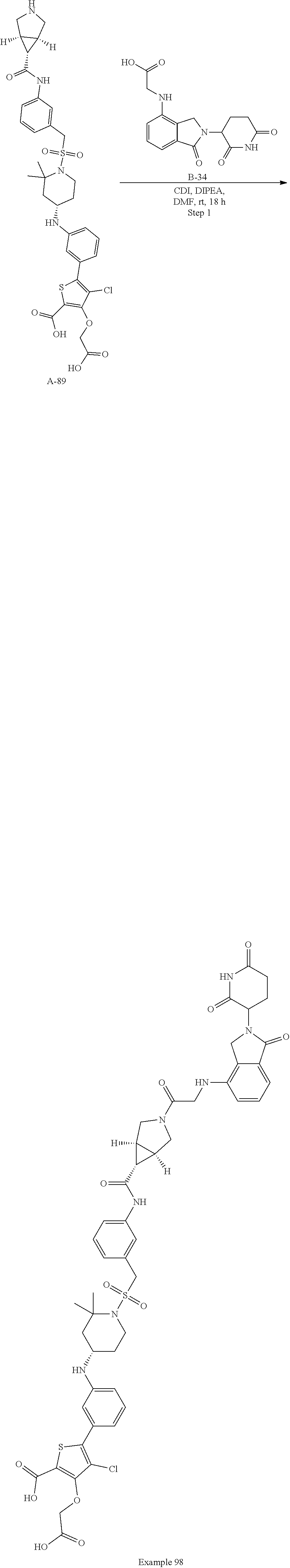

### Step 1: 3-(carboxymethoxy)-4-chloro-5-[3-[[(4S)-1-[3-[(1S,5R)-3-[2-[[2-(2,6-dioxo-3-piperidyl)-1-oxo-isoindolin-4-yl]amino]acetyl]-3-azabicyclo[3.1.0]hexane-6-carbonyl]amino]phenyl]methylsulfonyl]-2,2-dimethyl-4-piperidyl]amino]phenyl]thiophene-2-carboxylic acid (98)

3-(carboxymethoxy)-4-chloro-5-[3-[[(4S)-1-[3-[(1S,5R)-3-[2-[[2-(2,6-dioxo-3-piperidyl)-1-oxo-isoindolin-4-yl]amino]acetyl]-3-azabicyclo[3.1.0]hexane-6-carbonyl]amino]phenyl]methylsulfonyl]-2,2-dimethyl-4-piperidyl]amino]phenyl]thiophene-2-carboxylic acid (98, 5.7 mg, 4.51 μmol, 8% yield, TFA salt) was synthesized from 2-[[2-(2,6-dioxo-3-piperidyl)-1-oxo-isoindolin-4-yl]amino]acetic acid (B-34) and 5-[3-[[(4S)-1-[3-[(1S,5R)-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-89) in a similar fashion to Example 23, except using excess DIPEA as base and DMF as the solvent. The residue was purified by reverse phase preparative HPLC [Purification method: X Bridge C18, 19×150 mm, 5 μm; Mobile phase A: 0.1% TFA in water and Mobile phase B: MeCN]. LCMS (ES+): m/z 1016.2 [M+H]⁺. ¹H NMR (400 MHz, DMSO-d6): 11.01 (s, 1H), 10.29 (s, 1H), 7.71 (s, 1H), 7.53 (d, J=8.00 Hz, 1H), 7.33-7.28 (m, 2H), 7.23-7.19 (m, 1H), 7.08 (d, J=7.60 Hz, 1H), 7.00 (d, J=7.20 Hz, 1H), 6.99 (s, 1H), 6.82 (d, J=7.60 Hz, 1H), 6.74-6.69 (m, 2H), 5.12 (dd, J=4.80, 13.20 Hz, 1H), 4.92 (s, 2H), 4.40 (d, J=14.00 Hz, 1H), 4.33-4.26 (m, 2H), 4.18 (d, J=16.80 Hz, 1H), 4.02-3.98 (m, 1H), 3.91-3.85 (m, 2H), 3.68-3.78 (m, 2H), 3.16-3.04 (m, 2H), 2.97-2.88 (m, 1H), 2.70-2.59 (m, 2H), 2.40-2.31 (m, 2H), 2.21-2.13 (m, 1H), 2.07-2.02 (m, 2H), 1.91-1.82 (m, 1H), 1.80-1.72 (m, 1H), 1.68-1.62 (m, 1H), 1.48-1.32 (m, 7H), 1.15-1.06 (m, 1H).

### Example 99

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-4-piperidyl]amino]phenyl]thiophene-2-carboxylic acid (99)

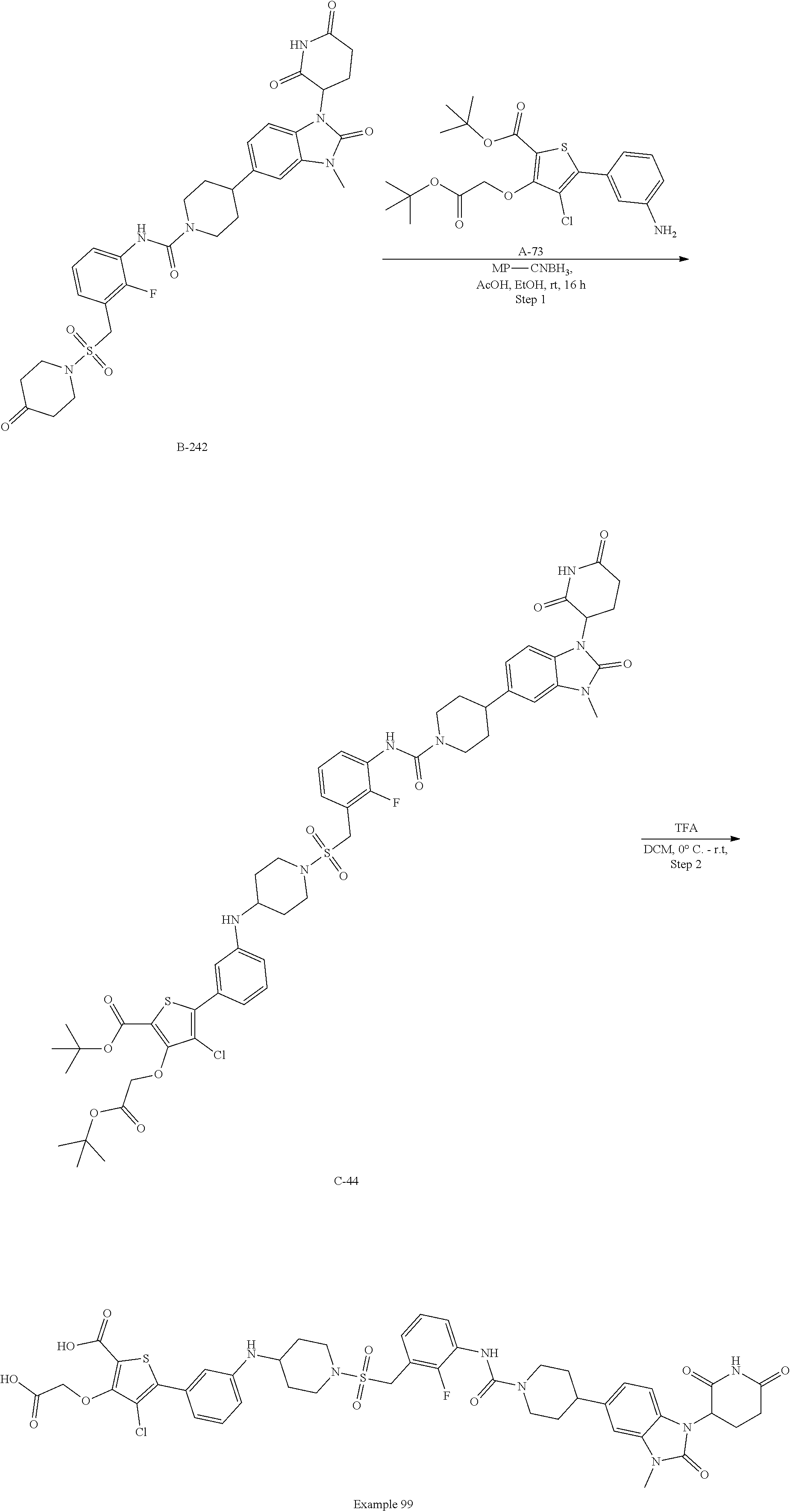

### Step 1: tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-S-yl]piperidine-1-carbonyl]amino]-2-fluoro-phenyl]methylsulfonyl]-4-piperidyl]amino]phenyl]thiophene-2-carboxylate (C-44)

3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-4-piperidyl]amino]phenyl]thiophene-2-carboxylate (C-44, 70 mg, 0.041 mmol, 77% yield) was synthesized from 4-[1-(2,6-dioxo-3-piperidyl)-3-methyl-2-oxo-benzimidazol-5-yl]-N-[2-fluoro-3-[(4-oxo-1-piperidyl)sulfonylmethyl]phenyl]piperidine-1-carboxamide (B-242) and tert-butyl 5-(3-aminophenyl)-3-(2-tert-butoxy-2-oxo-ethoxy)-4-chloro-thiophene-2-carboxylate (A-73) in a similar fashion to Compound C-38. LCMS (ESI): m/z 1100.1 [M+Na]⁺.

### Step 2: 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-4-piperidyl]amino]phenyl]thiophene-2-carboxylic acid (99)

3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-4-piperidyl]amino]phenyl]thiophene-2-carboxylic acid (99, 22 mg, 0.020 mmol, 49% yield) was synthesized from tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-4-piperidyl]amino]phenyl]thiophene-2-carboxylate (C-44) in a similar fashion to Compound C-1, except using 160 eq. of TEA. The residue was purified by reverse phase HPLC following the method: C₁₈ column X BRIDGE (19×150 mm; 5 μm) with solvent A: 0.1% TFA in water; Solvent B: Acetonitrile; Flow rate: 15 mL/min. LCMS (ESI): m/z 966.0 [M+H]⁺. ¹H NMR (400 MHz, DMSO-d₆) δ 11.09 (s, 1H), 8.43 (s, 1H), 7.45-7.39 (m, 1H), 7.24-7.10 (m, 5H), 7.02 (d, J=8.1 Hz, 1H), 6.96-6.91 (m, 1H), 6.90-6.87 (m, 1H), 6.83-6.79 (m, 1H), 6.74-6.70 (m, 1H), 5.34 (dd, J=12.7, 5.4 Hz, 1H), 4.90 (s, 2H), 4.44 (s, 2H), 4.27 (d, J=12.7 Hz, 2H), 3.33 (s, 3H), 3.00 (t, J=11.2 Hz, 2H), 2.95-2.84 (m, 3H), 2.81-2.58 (m, 4H), 2.04-1.88 (m, 4H), 1.83-1.76 (m, 2H), 1.71-1.57 (m, 2H), 1.41-1.28 (m, 2H).

### Example 100

### 5-[3-[[(4S)-1-[3-[(3aR,6aS)-2-[2-[[2-(2,6-dioxo-3-piperidyl)-1-oxo-isoindolin-4-yl]oxyacetyl]-3,3a,4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (100)

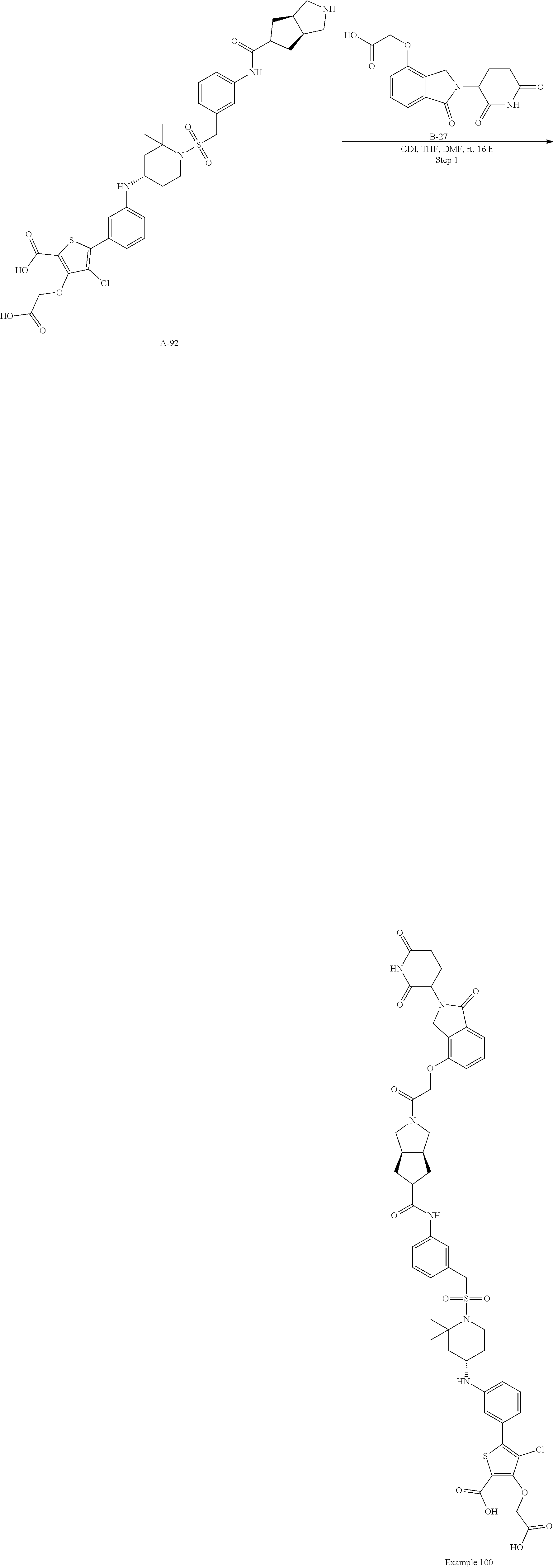

### Step 1:5-[3-[[(4S)-1-[3-[(3aR,6aS)-2-[2-[[2-(2,6-dioxo-3-piperidyl)-1-oxo-isoindolin-4-yl]oxyacetyl]-3,3a,4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (100)

5-[3-[[(4S)-1-[[3-[[(3aR,6aS)-2-[2-[[2-(2,6-dioxo-3-piperidyl)-1-oxo-isoindolin-4-yl]oxyacetyl]-3,3a, 4,5,6,6a-hexahydro-1H-cyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (100, 9 mg, 7.75 μmol, 12% yield, TFA salt) was synthesized from 2-[[2-(2,6-dioxo-3-piperidyl)-1-oxo-isoindolin-4-yl]oxyacetic acid (B-27) and 5-[3-[[(4S)-1-[3-[(3aR,6aS)-1,2,3,3a,4,5,6,6a-octahydrocyclopenta[c]pyrrole-5-carbonyl]amino]phenyl]methylsulfonyl]-2,2-dimethyl-4-piperidyl]amino]phenyl]-3-(carboxymethoxy)-4-chloro-thiophene-2-carboxylic acid (A-92) in a similar fashion to Example 23, except using THE: DMF as the solvent. The residue was purified by reverse phase prep HPLC [Purification method: Column: XSelect, C18 (150×19) mm, 5 μm; Mobile phase A: 0.1% TFA in water and Mobile phase B: MeCN).] LCMS (ES+): m/z 1046.7 [M+H]⁺. ¹H NMR (400 MHz, DMSO-d₆) δ 10.98 (s, 1H), 9.98 (s, 1H), 7.66 (s, 1H), 7.62 (d, J=8.5 Hz, 1H), 7.44 (t, J=7.8 Hz, 1H), 7.33-7.27 (m, 2H), 7.20 (t, J=7.9 Hz, 1H), 7.14 (d, J=8.2 Hz, 1H), 7.07 (d, J=7.7 Hz, 1H), 6.87 (s, 1H), 6.81 (d, J=7.4 Hz, 1H), 6.71-6.66 (m, 1H), 5.10 (dd, J=13.2, 5.1 Hz, 1H), 4.96-4.84 (m, 4H), 4.45-4.35 (m, 2H), 4.32-4.22 (m, 2H), 3.66-3.60 (m, 2H), 3.16-3.08 (m, 2H), 3.02-2.93 (m, 2H), 2.92-2.83 (m, 1H), 2.80-2.70 (m, 2H), 2.64-2.58 (m, 3H), 2.20-2.10 (m, 2H), 2.03-1.95 (m, 1H), 1.91-1.84 (m, 1H), 1.82-1.74 (m, 1H), 1.74-1.55 (m, 2H), 1.46 (s, 3H), 1.43-1.34 (m, 4H), 1.17-1.04 (m, 1H).

### Example 101

### 3-(carboxymethoxy)-4-chloro-5-[3-[[(4S)-1-[3-[(1S,5R)-3-[2-[[2-(2,6-dioxo-3-piperidyl)-1-oxo-isoindolin-4-yl]oxyacetyl]-3-azabicyclo[3.1.0]hexane-6-carbonyl]amino]phenyl]methylsulfonyl]-2,2-dimethyl-4-piperidyl]amino]phenyl]thiophene-2-carboxylic acid (101)

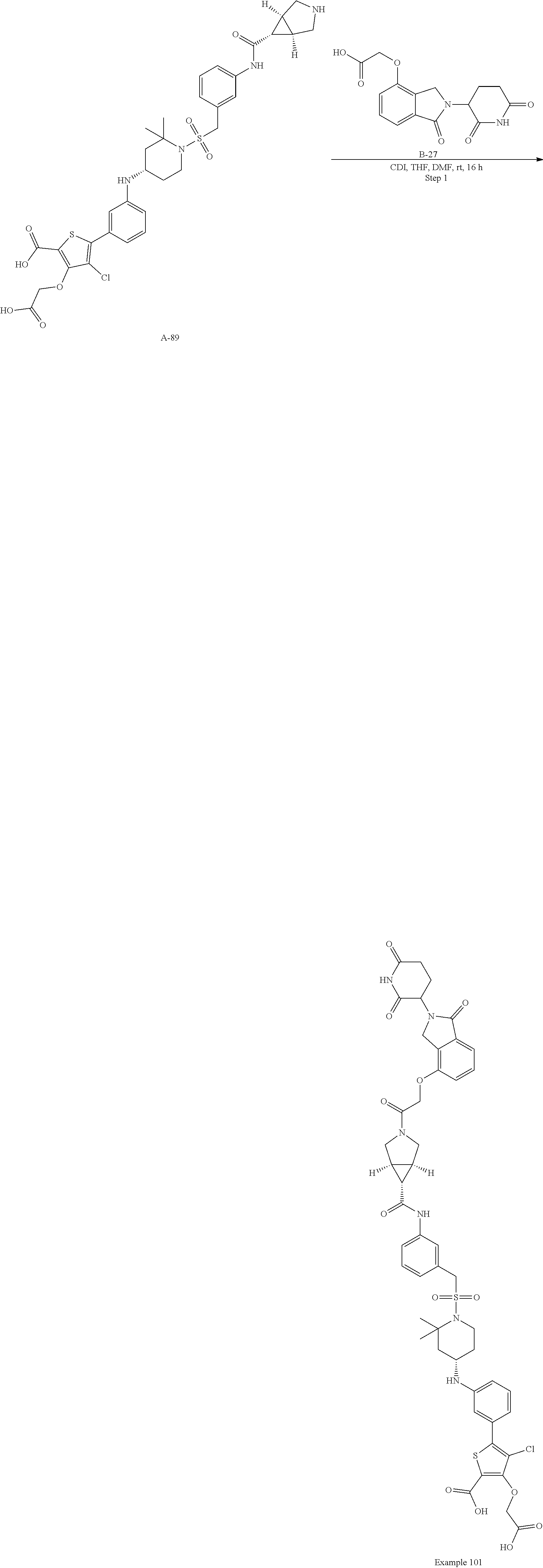

### Step 1: 3-(carboxymethoxy)-4-chloro-5-[3-[[(4S)-1-[[3-[[(1S,5R)-3-[2-[[2-(2,6-dioxo-3-piperidyl)-1-oxo-isoindolin-4-yl]oxyacetyl]-3-azabicyclo[3.1.0]hexane-6-carbonyl]amino]phenyl]methylsulfonyl]-2,2-dimethyl-4-piperidyl]amino]phenyl]thiophene-2-carboxylic acid (101)

3-(carboxymethoxy)-4-chloro-5-[3-[[(4S)-1-[[3-[(1S,5R)-3-[2-[[2-(2,6-dioxo-3-piperidyl)-1-oxo-isoindolin-4-yl]oxyacetyl]-3-azabicyclo[3.1.0]hexane-6-carbonyl]amino]phenyl]methylsulfonyl]-2,2-dimethyl-4-piperidyl]amino]phenyl]thiophene-2-carboxylic acid (101, 10 mg, 8.68 μmol, 14% yield, TFA salt) was synthesized from 2-[[2-(2,6-dioxo-3-piperidyl)-1-oxo-isoindolin-4-yl]oxyacetic acid (B-27) and 5-[3-[[(4S)-1-[3-[(1S,5R)-3-azabicyclo[3.1.0]hexane-6-carbonyl]amino]phenyl]methylsulfonyl]-2,2-dimethyl-4-piperidyl]amino]phenyl]-3-(carboxymethoxy)-4-chloro-thiophene-2-carboxylic acid (A-89) in a similar fashion to Example 23, except using THF:DMF as the solvent. The residue was purified by reverse phase prep HPLC [Purification method: Column: XSelect, C18 (150×19 mm), 5 μm; Mobile phase A: 0.1% TFA in water and Mobile phase B: MeCN). LCMS (ES+): m/z 1018.0 [M+H]⁺. ¹H NMR (400 MHz, DMSO-d6): δ 11.00 (d, 1H), 10.30 (d, 1H), 7.71 (s, 1H), 7.54-7.45 (m, 1H), 7.35-7.29 (m, 2H), 7.23-7.07 (m, 3H), 6.87 (s, 1H), 6.82 (d, J=7.60 Hz, 1H), 6.70 (d, J=8.00 Hz, 1H), 5.11-5.09 (m, 1H), 4.93-4.75 (m, 4H), 4.42-4.27 (m, 4H), 3.83-3.68 (m, 4H), 3.17-3.09 (m, 2H), 2.91-2.81 (m, 2H), 2.34-2.33 (m, 1H), 2.04-2.01 (m, 2H), 1.89-1.67 (m, 3H), 1.45-1.38 (m, 7H), 1.24-1.24 (m, 1H).

### Example 102

### 3-(carboxymethoxy)-4-chloro-5-[3-[[1-[[3-[[7-[1-(2,6-dioxo-3-piperidyl)-3-methyl-2-oxo-benzimidazol-5-yl]-2,7-diazaspiro[3.5]nonane-2-carbonyl]amino]phenyl]methylsulfonyl]-4-piperidyl]amino]phenyl]thiophene-2-carboxylic acid (102)

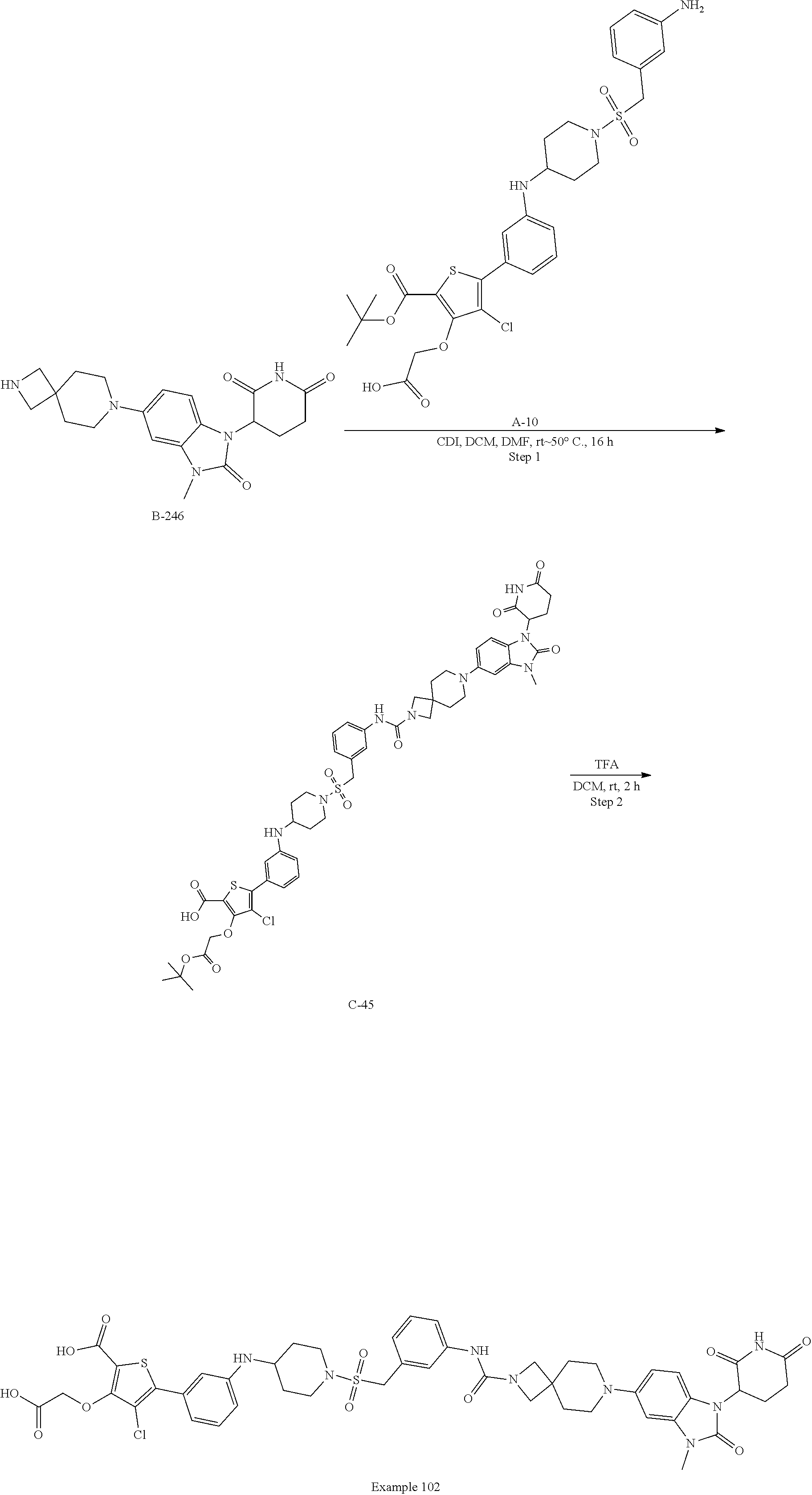

### Step 1: 3-(2-tert-butoxy-2-oxo-ethoxy)-4-chloro-5-[3-[[1-[[3-[[7-[1-(2,6-dioxo-3-piperidyl)-3-methyl-2-oxo-benzimidazol-5-yl]-2,7-diazaspiro[3.5]nonane-2-carbonyl]amino]phenyl]methylsulfonyl]-4-piperidyl]amino]phenyl]thiophene-2-carboxylic acid (C-45)

3-(2-tert-butoxy-2-oxo-ethoxy)-4-chloro-5-[3-[[1-[[3-[7-[1-(2,6-dioxo-3-piperidyl)-3-methyl-2-oxo-benzimidazol-5-yl]-2,7-diazaspiro[3.5]nonane-2-carbonyl]amino]phenyl]methylsulfonyl]-4-piperidyl]amino]phenyl]thiophene-2-carboxylic acid (C-45, 11 mg, 9.01 μmol, 6% yield, TFA salt) was synthesized from 2-[[5-[3-[1]-[(3-aminophenyl)methylsulfonyl]-4-piperidyl]amino]phenyl]-2-tert-butoxycarbonyl-4-chloro-3-thienyl]oxy]acetic acid (A-10) and 3-[5-(2,7-diazaspiro[3.5]nonan-7-yl)-3-methyl-2-oxo-benzimidazol-1-yl]piperidine-2,6-dione (B-246) in a similar fashion to Example 23, except using DCM:DMF as the solvent and with heating to 50° C. The residue was purified by reverse phase prep HPLC [Purification method: Column: XSelect, C18 (150×19 mm), 5 μm; Mobile phase A: 0.1% TFA in water and Mobile phase B: MeCN)]. UPLC-MS (ES+): m/z 1045.3 [M+H]⁺.

### Step 2: 3-(carboxymethoxy)-4-chloro-5-[3-[[1-[[3-[[7-[1-(2,6-dioxo-3-piperidyl)-3-methyl-2-oxo benzimidazol-5-yl]-2,7-diazaspiro[3.5]nonane-2-carbonyl]amino]phenyl]methylsulfonyl]-4-piperidyl]amino]phenyl]thiophene-2-carboxylic acid (102)

3-(carboxymethoxy)-4-chloro-5-[3-[[1-[[3-[[7-[1-(2,6-dioxo-3-piperidyl)-3-methyl-2-oxo-benzimidazol-5-yl]-2,7-diazaspiro[3,5]nonane-2-carbonyl]amino]phenyl]methylsulfonyl]-4-piperidyl]amino]phenyl]thiophene-2-carboxylic acid (102, 6.00 mg, 5.08 μmol, 13% yield, TFA salt) was synthesized from 3-(2-tert-butoxy-2-oxo-ethoxy)-4-chloro-5-[3-[[1-[[3-[7-[1-(2,6-dioxo-3-piperidyl)-3-methyl-2-oxo-benzimidazol-5-yl]-2,7-diazaspiro[3.5]nonane-2-carbonyl]amino]phenyl]methylsulfonyl]-4-piperidyl]amino]phenyl]thiophene-2-carboxylic acid (C-45) in a similar fashion to Compound C-1, except using 10 eq. of TFA. The final product was triturated from diethyl ether. LCMS (ES+): m/z 989.0 [M+H]⁺. ¹H NMR (400 MHz, DMSO-d₆): δ 11.07 (s, 1H), 8.53 (s, 1H), 7.47 (d, J=7.20 Hz, 1H), 7.26-7.19 (m, 2H), 7.00-6.94 (m, 2H), 6.87-6.80 (m, 3H), 6.72-6.70 (m, 2H), 5.88-5.85 (m, 1H), 5.32-5.28 (m, 1H), 4.92-4.91 (m, 3H), 4.34 (s, 2H), 3.73-3.51 (m, 8H), 3.32-2.86 (m, 6H), 2.33-1.92 (m, 6H), 1.80-0.20 (m, 2H).

### Example 103

### 3-(Carboxymethoxy)-4-chloro-5-[3-[[1-[[3-[[4-[1-(2,6-dioxo-3-piperidyl)-3-methyl-2-oxo-benzimidazol-S-yl]piperidine-1-carbonyl]amino]phenyl]methylsulfonyl]-4-piperidyl]oxy]phenyl]thiophene-2-carboxylic acid (103)

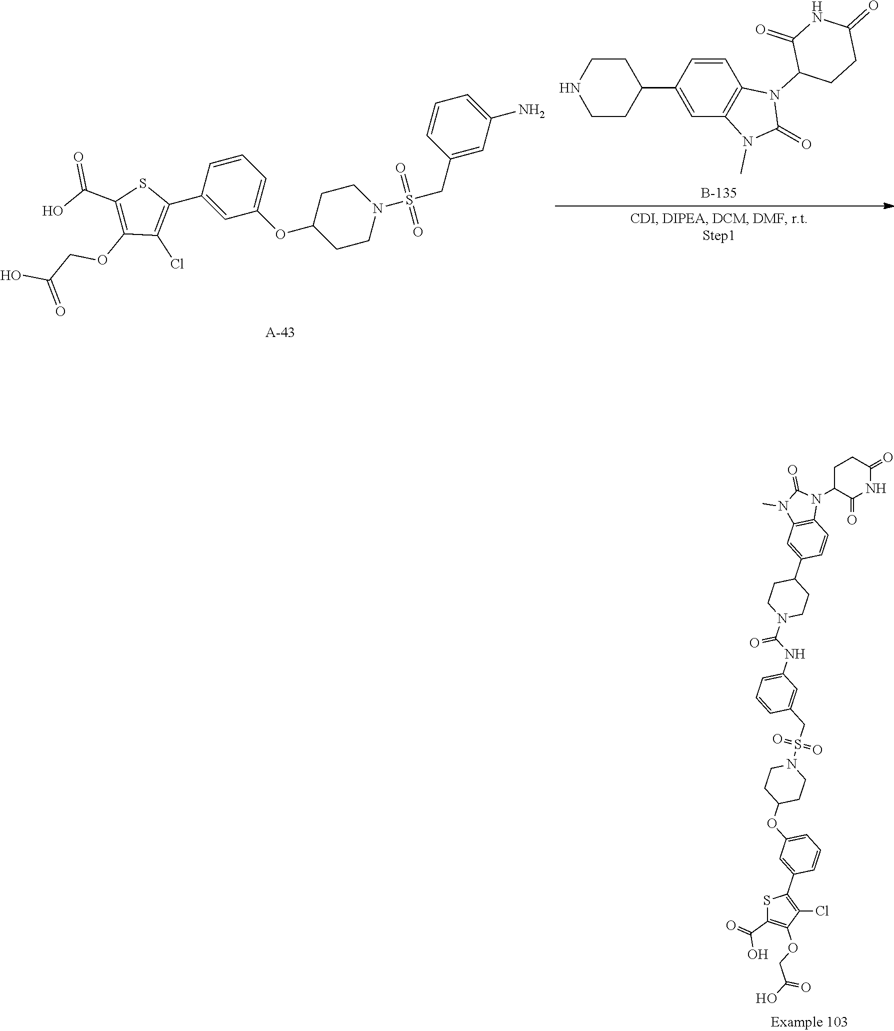 [SMILES: Nc1cccc(CS(=O)(=O)N2CCC(Oc3cccc(-c4sc(C(=O)O)c(OCC(=O)O)c4Cl)c3)CC2)c1 ; Cn1c(=O)n(C2CCC(=O)NC2=O)c2ccc(C3CCNCC3)cc21 ; "A-43" ; Cn1c(=O)n(C2CCC(=O)NC2=O)c2ccc(C3CCN(C(=O)Nc4cccc(CS(=O)(=O)N5CCC(Oc6cccc(-c7sc(C(=O)O)c(OCC(=O)O)c7Cl)c6)CC5)c4)CC3)cc21]

### Step 1: 3-(Carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]oxy]phenyl]thiophene-2-carboxylic acid (103)

3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]oxy]phenyl]thiophene-2-carboxylic acid (103, 9.0 mg, 0.0094 mmol, 15% yield) was synthesized from 5-[3-[[1-[(3-aminophenyl)methylsulfonyl]-4-piperidyl]oxy]phenyl]-3-(carboxymethoxy)-4-chloro-thiophene-2-carboxylic acid (A-43) and 3-[3-methyl-2-oxo-5-(4-piperidyl)benzimidazol-1-yl]piperidine-2,6-dione (B-135) in a similar fashion to Example 23, except using excess DIPEA as base and DCM:DMF as the solvent. The residue was purified by reverse phase HPLC with the method containing SunFire C18 (19×150 mm, 5 μm), Mobile phase A: 0.1% TFA in water and B: Acetonitrile, Flow rate=15 mL/min. LCMS (ES+): m/z 948.5 [M+H]⁺. ¹H NMR (400 MHz, DMSO-d₆) δ 11.09 (s, 1H), 8.63 (s, 1H), 7.63-7.60 (m, 1H), 7.47-7.41 (m, 2H), 7.27-7.21 (m, 3H), 7.14-7.10 (m, 2H), 7.03-6.98 (m, 2H), 6.95-6.90 (m, 1H), 5.34 (dd, J=12.8, 5.4 Hz, 1H), 4.93 (s, 2H), 4.71-4.60 (m, 1H), 4.37 (s, 2H), 4.30 (d, J=12.9 Hz, 2H), 3.33 (s, 3H), 3.16 (ddt, J=11.9, 7.9, 3.7 Hz, 3H), 2.95-2.82 (m, 3H), 2.81-2.73 (m, 1H), 2.72-2.57 (m, 3H), 2.04-1.90 (m, 3H), 1.79 (d, J=12.3 Hz, 2H), 1.73-1.54 (m, 4H).

### Example 104

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-4-piperidyl]amino]phenyl]thiophene-2-carboxylic acid (104)

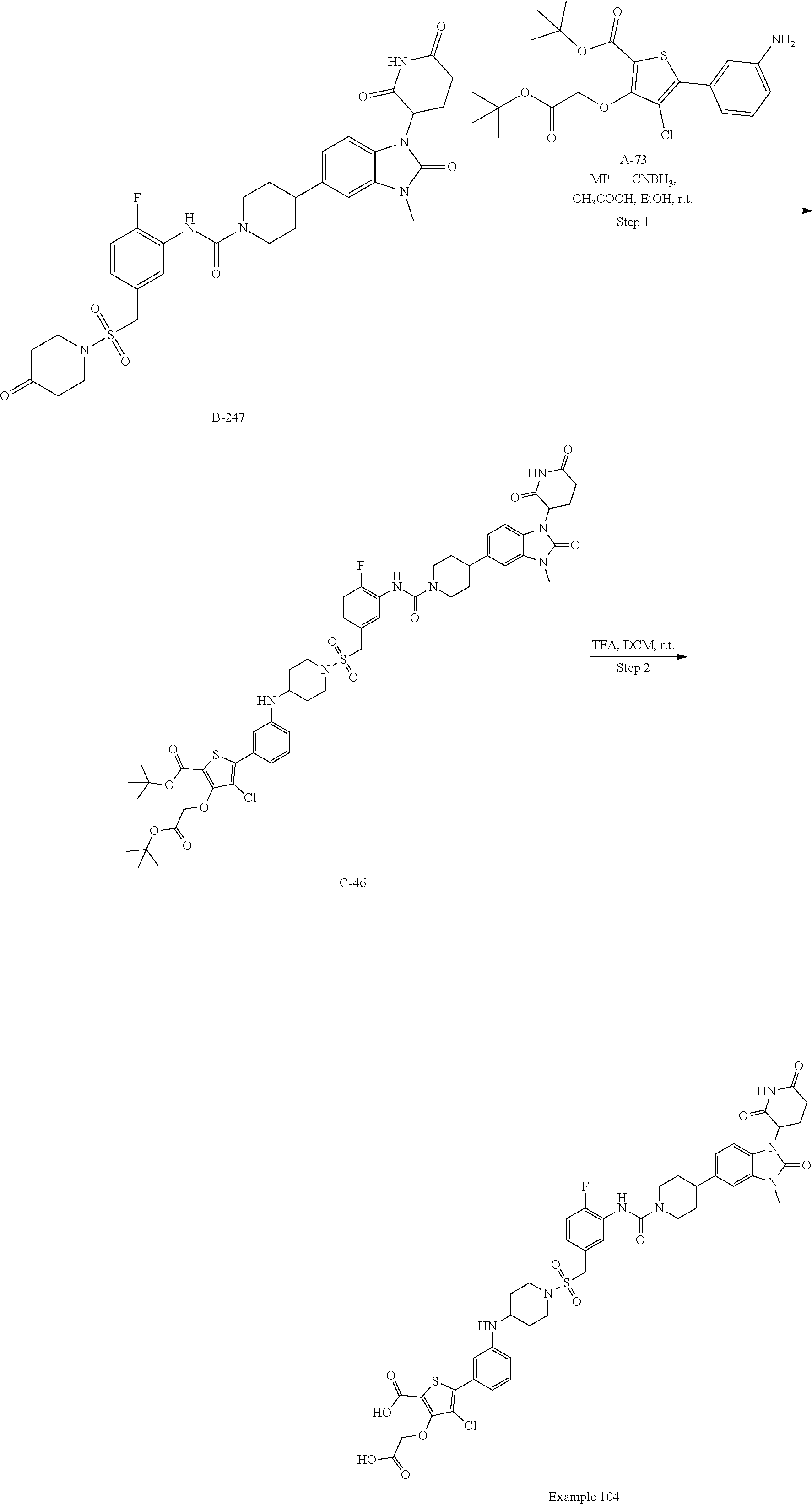

### Step 1: tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-4-piperidyl]amino]phenyl]thiophene-2-carboxylate (C-46)

Tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-4-piperidyl]amino]phenyl]thiophene-2-carboxylate (C-46, 85 mg, 0.032 mmol, 85% yield, 40% purity) was synthesized from 4-[1-(2,6-dioxo-3-piperidyl)-3-methyl-2-oxo-benzimidazol-5-yl]-N-[2-fluoro-5-[(4-oxo-1-piperidyl)sulfonylmethyl]phenyl]piperidine-1-carboxamide (B-247) and tert-butyl 5-(3-aminophenyl)-3-(2-tert-butoxy-2-oxo-ethoxy)-4-chloro-thiophene-2-carboxylate (A-73) in a similar fashion to Compound C-38. LCMS (ES+): m/z 1077.9 [M+H]⁺.

### Step 2: 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-4-piperidyl]amino]phenyl]thiophene-2-carboxylic acid (104)

3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-4-piperidyl]amino]phenyl]thiophene-2-carboxylic acid (104, 15 mg, 0.013 mmol, 46% yield) was synthesized from tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-4-piperidyl]amino]phenyl]thiophene-2-carboxylate (C-46) in a similar fashion to Compound C-1, except using 130 eq. TFA. The residue was purified by reverse phase HPLC using the method: Column: XSELECT (150×19 mm) 5 μm; Mobile phase: 0.1% TFA in water and MeCN; Flow rate: 15 mL/min. LCMS (ES−): m/z 964.0 [M−H]⁻. ¹H NMR (400 MHz, DMSO-d₆) δ 11.09 (s, 1H), 8.39 (s, 1H), 7.55 (dd, J=7.6, 2.2 Hz, 1H), 7.25-7.18 (m, 2H), 7.18-7.13 (m, 1H), 7.12-7.10 (m, 1H), 7.02 (d, J=8.1 Hz, 1H), 6.95-6.90 (m, 1H), 6.90-6.87 (m, 1H), 6.84-6.80 (m, 1H), 6.75-6.70 (m, 1H), 5.34 (dd, J=12.7, 5.4 Hz, 1H), 4.90 (s, 2H), 4.40 (s, 2H), 4.27 (d, J=12.7 Hz, 2H), 3.33 (s, 3H), 3.01-2.83 (m, 6H), 2.82-2.57 (m, 4H), 2.05-1.97 (m, 1H), 1.97-1.89 (m, 2H), 1.79 (d, J=12.5 Hz, 2H), 1.70-1.55 (m, 2H), 1.39-1.27 (m, 2H).

### Example 105

### 3-(carboxymethoxy)-4-chloro-5-[3-[[(4S)-1-[[3-[[2-[[1-[1-(2,6-dioxo-3-piperidyl)-3-methyl-2-oxo-benzimidazol-5-yl]-4-piperidyl]oxy]acetyl]amino]phenyl]methylsulfonyl]-2,2-dimethyl-4-piperidyl]amino]phenyl]thiophene-2-carboxylic acid (105)

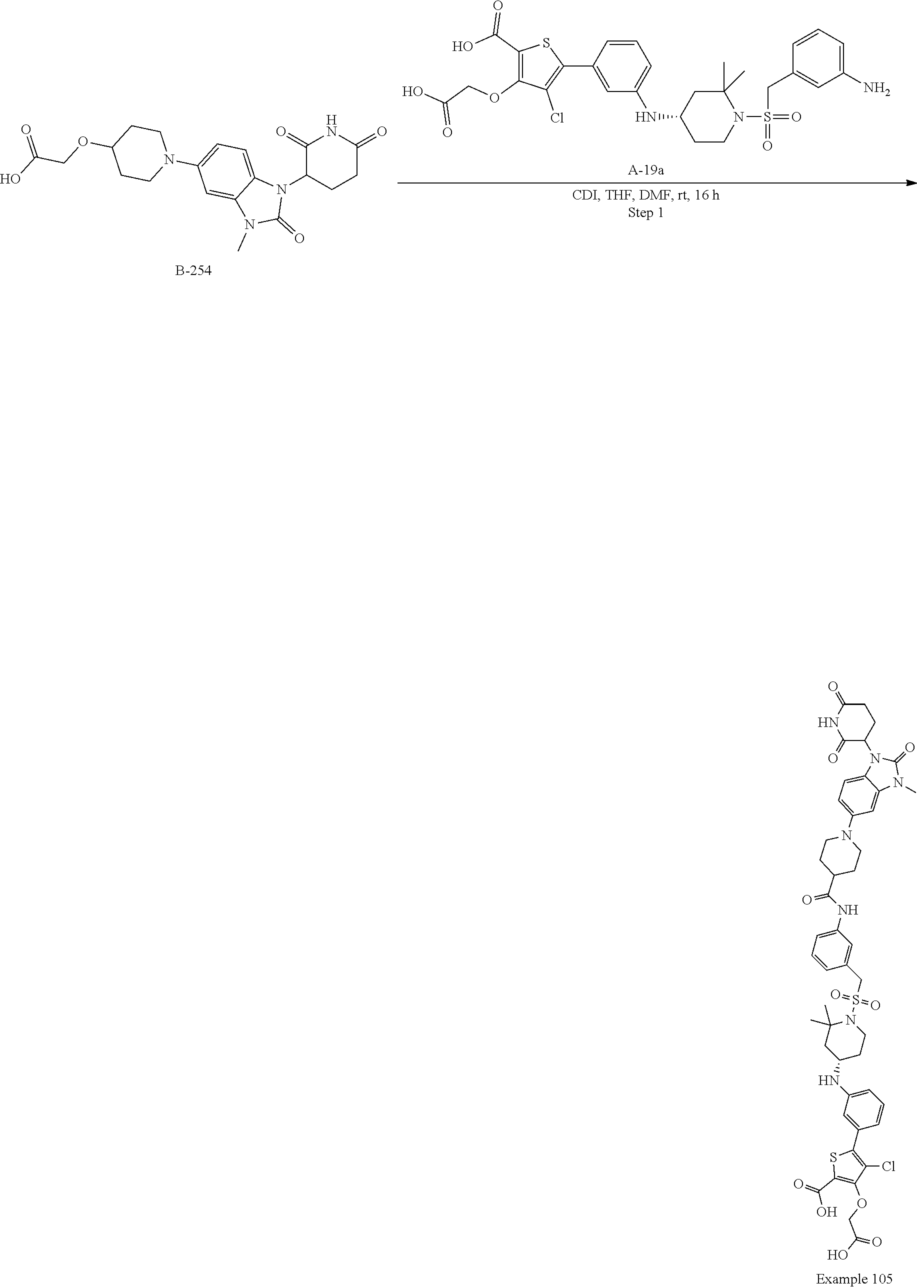 [SMILES: Cn1c(=O)n(C2CCC(=O)NC2=O)c2ccc(N3CCC(OCC(=O)O)CC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(N3CCC(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[3-[2-[[1-[1-(2,6-dioxo-3-piperidyl)-3-methyl-2-oxo-benzimidazol-5-yl]-4-piperidyl]oxy]acetyl]amino]phenyl]methylsulfonyl]-2,2-dimethyl-4-piperidyl]amino]phenyl]thiophene-2-carboxylic acid (105)

3-(carboxymethoxy)-4-chloro-5-[3-[[(4S)-1-[3-[2-[1-[1-(2,6-dioxo-3-piperidyl)-3-methyl-2-oxo-benzimidazol-5-yl]-4-piperidyl]oxy]acetyl]amino]phenyl]methylsulfonyl]-2,2-dimethyl-4-piperidyl]amino]phenyl]thiophene-2-carboxylic acid (105, 10 mg, 8.83 μmol, 12% yield, TFA salt) was synthesized from 2-[[1-[1-(2,6-dioxo-3-piperidyl)-3-methyl-2-oxo-benzimidazol-5-yl]-4-piperidyl]oxy]acetic acid (B-254) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except for using THF:DMF as the solvent. The residue was purified by reverse phase prep HPLC [Purification method: Column: X BRIDGE C18 (19×150 mm), 5 μm, Mobile phase A: 0.1% TFA in water; Mobile phase B: MeCN; Flow Rate: 15.0 mL/min]. LCMS (ES+): m/z 1005.5 [M+H]⁺. ¹H NMR (400 MHz, DMSO-d6): δ 11.11 (s, 1H), 9.81 (s, 1H), 7.77 (s, 1H), 7.63 (d, J=7.60 Hz, 1H), 7.36-7.33 (m, 1H), 7.30-7.12 (m, 4H), 7.00 (s, 1H), 6.88-6.80 (m, 2H), 6.70 (d, J=8.00 Hz, 1H), 5.38-5.34 (m, 1H), 4.92 (s, 2H), 4.44-4.28 (m, 2H), 4.19 (s, 2H), 2.95-2.86 (m, 7H), 2.72-2.65 (m, 3H), 2.12-1.77 (m, 8H), 1.46-1.39 (m, 7H), 1.20-1.00 (m, 1H).

### Example 106

### 3-(carboxymethoxy)-4-chloro-5-[3-[[(4S)-1-[[3-[[3-[1-(2,6-dioxo-3-piperidyl)-3-methyl-2-oxo-benzimidazol-5-yl]azetidine-1-carbonyl]amino]phenyl]methylsulfonyl]-2,2-dimethyl-4-piperidyl]amino]phenyl]thiophene-2-carboxylic acid (106)

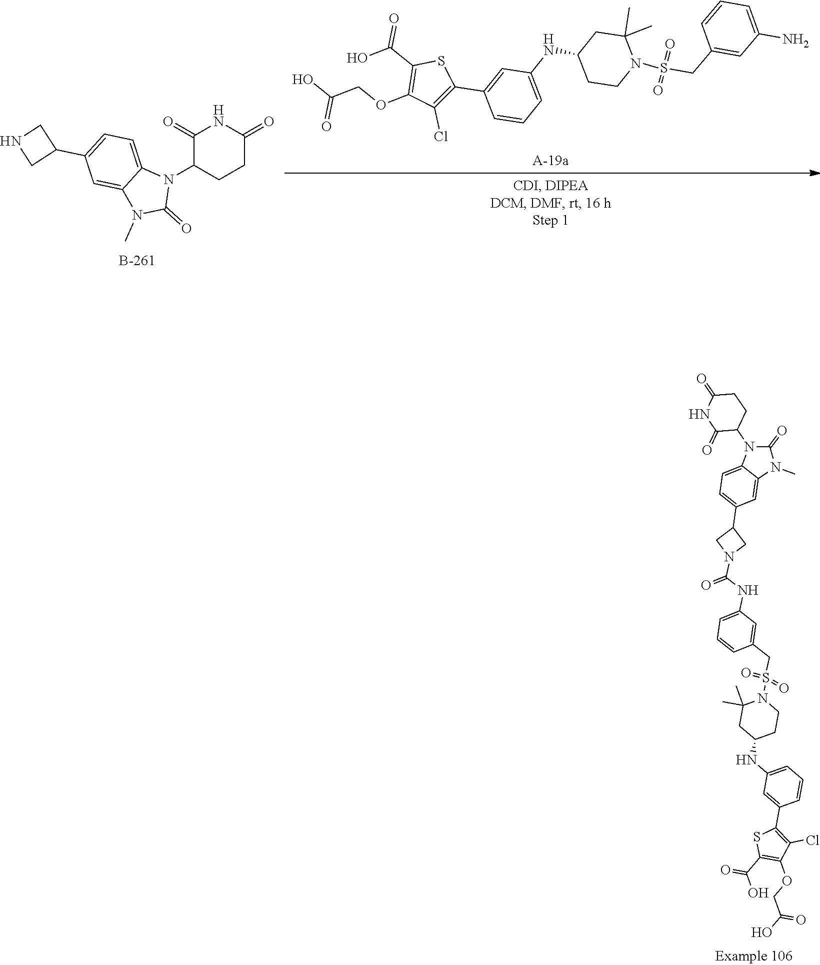 [SMILES: Cn1c(=O)n(C2CCC(=O)NC2=O)c2ccc(C3CNC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(C3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21]

### Step 1: 3-(carboxymethoxy)-4-chloro-5-[3-[[(4S)-1-[[3-[[3-[1-(2,6-dioxo-3-piperidyl)-3-methyl-2-oxo-benzimidazol-5-yl]azetidine-1-carbonyl]amino]phenyl]methylsulfonyl]-2,2-dimethyl-4-piperidyl]amino]phenyl]thiophene-2-carboxylic acid (106)

3-(carboxymethoxy)-4-chloro-5-[3-[[(4S)-1-[[3-[[3-[1-(2,6-dioxo-3-piperidyl)-3-methyl-2-oxo-benzimidazol-5-yl]azetidine-1-carbonyl]amino]phenyl]methylsulfonyl]-2,2-dimethyl-4-piperidyl]amino]phenyl]thiophene-2-carboxylic acid (106, 26 mg, 24.36 μmol, 33% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(azetidin-3-yl)-3-methyl-2-oxo-benzimidazol-1-yl]piperidine-2,6-dione (B-261) in a similar fashion to Example 23, except using excess DIPEA as base and DCM:DMF as solvent. The residue was purified by reverse phase prep HPLC (Column: XSelecta; Mobile phase A: 0.1% TFA in water, Mobile phase B: MeCN), MS (ES+): m/z 947.6 [M+H]⁺. ¹H NMR. (400 MHz, DMSO-d₆) δ 11.10 (s, 1H), 8.61 (s, 1H), 7.62-7.59 (m, 1H), 7.56-7.51 (m, 1H), 7.29-7.22 (m, 2H), 7.19 (t, J=7.9 Hz, 1H), 7.09 (d, J=8.1 Hz, 1H), 7.06-7.02 (m, 1H), 6.98 (d, J=7.7 Hz, 1H), 6.90-6.85 (m, 1H), 6.81 (d, J=7.6 Hz, 1H), 6.73-6.66 (m, 1H), 5.36 (dd, J=12.7, 5.4 Hz, 1H), 4.91 (s, 2H), 4.42-4.33 (m, 3H), 4.25 (d, J=13.8 Hz, 1H), 4.00 (t, J=7.7 Hz, 3H), 3.57-3.49 (m, 2H), 3.48-3.41 (m, 1H), 3.35 (s, 3H), 3.12 (t, J=12.6 Hz, 1H), 2.96-2.84 (m, 1H), 2.77-2.59 (m, 2H), 2.05-1.98 (m, 1H), 1.91-1.82 (m, 1H), 1.81-1.74 (m, 1H), 1.47 (s, 3H), 1.44-1.36 (m, 4H), 1.23 (s, 1H), 1.19-1.06 (m, 1H).

### Example 107

### 3-(carboxymethoxy)-4-chloro-5-[3-[[(4S)-1-[[3-[[2-[1-[1-(2,6-dioxo-3-piperidyl)-3-methyl-2-oxo-benzimidazol-5-yl]azetidin-3-yl]oxyacetyl]amino]phenyl]methylsulfonyl]-2,2-dimethyl-4-piperidyl]amino]phenyl]thiophene-2-carboxylic acid (107)

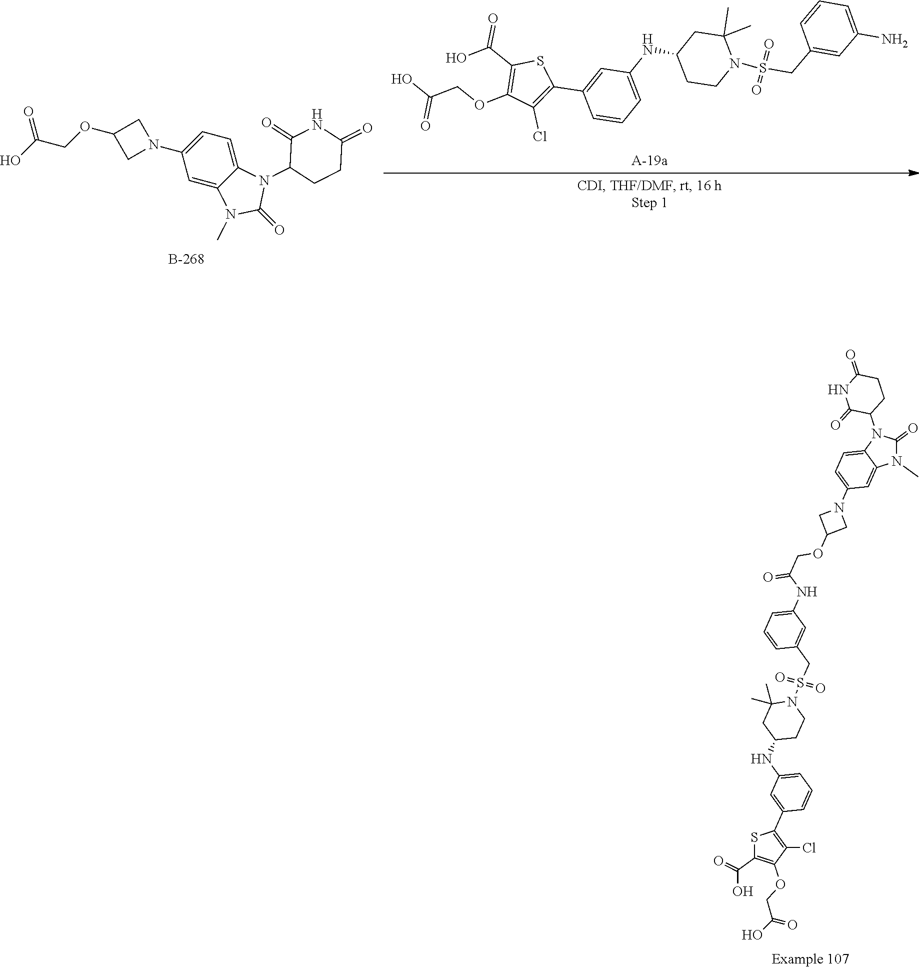 [SMILES: Cn1c(=O)n(C2CCC(=O)NC2=O)c2ccc(N3CC(OCC(=O)O)C3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(N3CC(OCC(=O)Nc4cccc(CS(=O)(=O)N5CC[C@H](Nc6cccc(-c7sc(C(=O)O)c(OCC(=O)O)c7Cl)c6)CC5(C)C)c4)C3)cc21]

### Step 1: 3-(carboxymethoxy)-4-chloro-5-[3-[[(4S)-1-[[3-[[2-[1-[1-(2,6-dioxo-3-piperidyl)-3-methyl-2-oxo-benzimidazol-5-yl]azetidin-3-yl]oxyacetyl]amino]phenyl]methylsulfonyl]-2,2-dimethyl-4-piperidyl]amino]phenyl]thiophene-2-carboxylic acid (107)

3-(carboxymethoxy)-4-chloro-5-[3-[[(4S)-1-[3-[2-[[1-[1-(2,6-dioxo-3-piperidyl)-3-methyl-2-oxo-benzimidazol-5-yl]azetidin-3-yl]oxyacetyl]amino]phenyl]methylsulfonyl]-2,2-dimethyl-4-piperidyl]amino]phenyl]thiophene-2-carboxylic acid (107, 3.5 mg, 2.78 μmol, 4% yield, TFA salt) was synthesized from 2-[1-[1-(2,6-dioxo-3-piperidyl)-3-methyl-2-oxo-benzimidazol-5-yl]azetidin-3-yl]oxyacetic acid (B-268) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 23, except using THE: DMF as a solvent. The residue which was subjected to reverse phase preparative HPLC (Column: XSelect C18 (150×19 mm) 5 μm, Mobile phase: 0.1% TFA in MQ water and acetonitrile). LCMS (ES+): m/z 978.5 [M+H]⁺. ¹H NMR (400 MHz, DMSO-d₆) δ 11.06 (s, 1H), 9.92 (s, 1H), 7.74 (s, 1H), 7.64-7.59 (m, 1H), 7.33 (t, J=7.8 Hz, 1H), 7.25-7.16 (m, 1H), 7.14-7.08 (m, 1H), 6.91 (d, J=8.4 Hz, 1H), 6.87 (s, 1H), 6.80 (d, J=7.6 Hz, 1H), 6.72-6.66 (m, 1H), 6.37 (d, J=2.1 Hz, 1H), 6.15 (dd, J=8.4, 2.2 Hz, 1H), 5.26 (dd, J=12.7, 5.5 Hz, 1H), 4.91 (s, 3H), 4.59-4.52 (m, 1H), 4.45-4.35 (m, 1H), 4.33-4.23 (m, 2H), 4.13 (s, 2H), 4.08 (t, J=7.1 Hz, 2H), 3.74-3.68 (m, 2H), 2.93-2.81 (m, 2H), 2.66-2.58 (m, 2H), 2.04-1.94 (m, 2H), 1.89-1.83 (m, 2H), 1.77 (d, J=12.5 Hz, 1H), 1.46 (s, 3H), 1.42-1.36 (m, 4H), 1.17-1.03 (m, 2H).

### Example 108

### 3-(Carboxymethoxy)-4-chloro-5-[3-[[(4R)-1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (108)

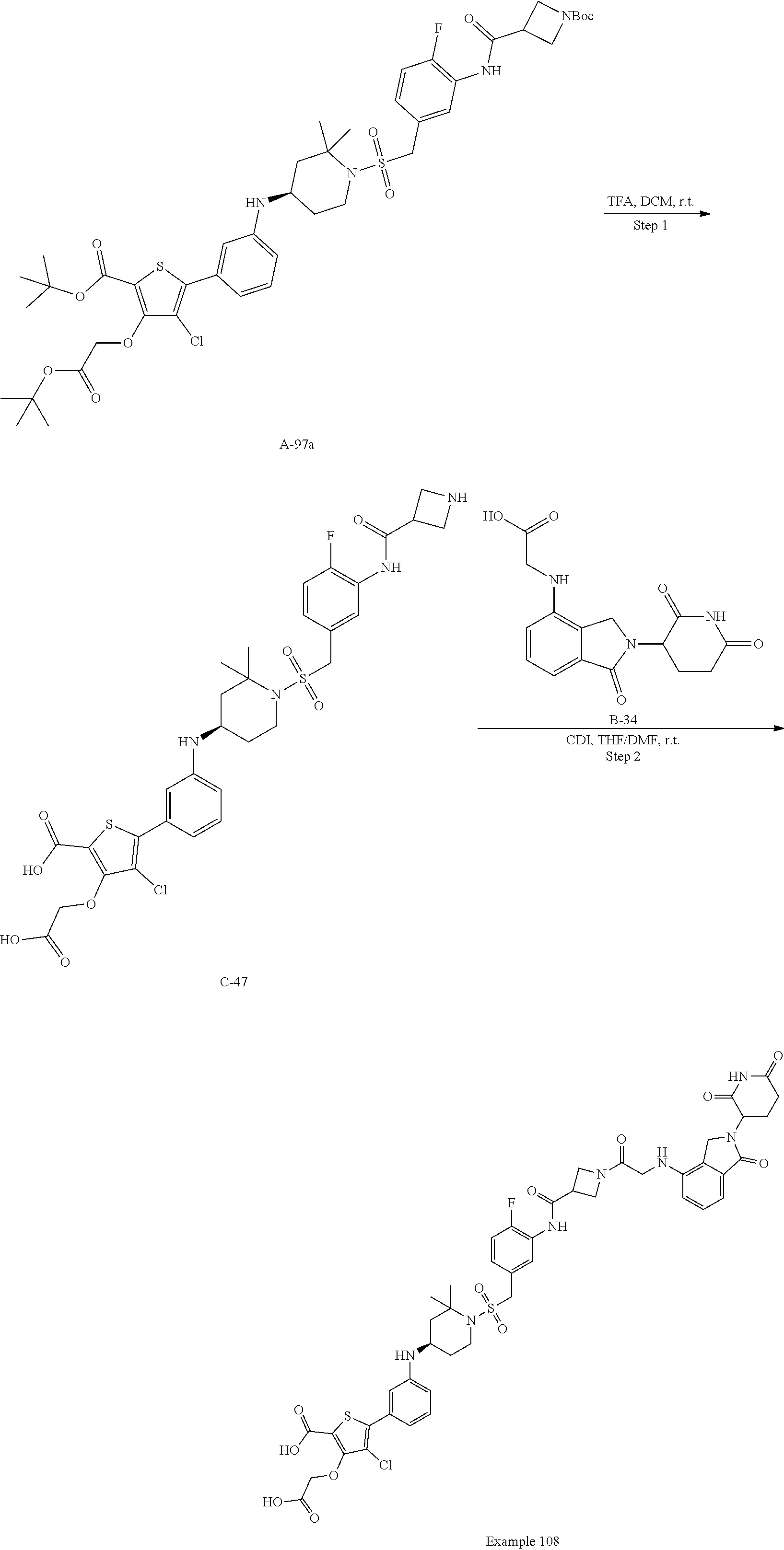

### Step 1:5-[3-[[(4R)-1-[[3-(azetidine-3-carbonylamino)-4-fluoro-phenyl]methylsulfonyl]-2,2-dimethyl-4-piperidyl]amino]phenyl]-3-(carboxymethoxy)-4-chloro-thiophene-2-carboxylic acid (C-47)

5-[3-[(4R)-1-[[3-(azetidine-3-carbonylamino)-4-fluoro-phenyl]methylsulfonyl]-2,2-dimethyl-4-piperidyl]amino]phenyl]-3-(carboxymethoxy)-4-chloro-thiophene-2-carboxylic acid (C-47, 85 mg, 0.099 mmol, 97% yield) was synthesized from tert-butyl 3-[[5-[(4R)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97a) in a similar fashion to Compound C-1, except using 130 eq. of TFA. LCMS (ESI): m/z 708.7 [M+H]⁺. ¹H NMR (400 MHz, DMSO-d₆) δ 10.03 (s, 1H), 8.73 (s, 1H), 8.09-8.06 (m, 1H), 7.33-7.19 (m, 4H), 6.87-6.80 (m, 2H), 6.70-6.68 (m, 1H), 4.92 (s, 3H), 4.47-4.31 (m, 2H), 4.12-4.07 (m, 4H), 3.92-3.88 (m, 1H), 1.90-1.79 (m, 2H), 1.54 (s, 2H), 1.43-1.37 (m, 6H) and 1.11-1.07 (m, 2H).

### Step 2: 3-(carboxymethoxy)-4-chloro-5-[3-[(4R)-1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (108)

3-(carboxymethoxy)-4-chloro-5-[3-[(4R)-1-[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (108, 21 mg, 0.018 mmol, 23% yield) was synthesized from 2-[[2-(2,6-dioxo-3-piperidyl)-1-oxo-isoindolin-4-yl]amino]acetic acid (B-34) and 5-[3-[[(4R)-1-[[3-(azetidine-3-carbonylamino)-4-fluoro-phenyl]methylsulfonyl]-2,2-dimethyl-4-piperidyl]amino]phenyl]-3-(carboxymethoxy)-4-chloro-thiophene-2-carboxylic acid (C-47) in a similar fashion to Example 23, except using THF:DMF as the solvent. The residue was purified by reverse phase HPLC following the method: Column: X-BRIDGE C8 (19×150 mm), 5 μm; Mobile phase A: 0.1% TFA in MQ water, B: Acetonitrile; flow rate: 15 mL/min. LCMS (ES+): m/z 1007.5 [M+H]⁺. ¹H NMR (400 MHz, DMSO-d₆) δ 11.00 (s, 1H), 9.97 (s, 1H), 8.02 (d, J=7.4 Hz, 1H), 7.33-7.25 (m, 2H), 7.20 (t, J=7.9 Hz, 2H), 6.98 (d, J=7.4 Hz, 1H), 6.88 (s, 1H), 6.81 (d, J=7.6 Hz, 1H), 6.74-6.63 (m, 2H), 5.11 (dd, J=13.2, 5.1 Hz, 1H), 4.91 (s, 2H), 4.47-4.38 (m, 2H), 4.37-4.31 (m, 2H), 4.28 (d, J=17.4 Hz, 1H), 4.16 (d, J=17.1 Hz, 1H), 4.11-3.98 (m, 4H), 3.17-3.07 (m, 2H), 2.98-2.85 (m, 2H), 2.68-2.57 (m, 2H), 2.40-2.27 (m, 2H), 2.08-1.97 (m, 1H), 1.93-1.85 (m, 1H), 1.79 (d, J=12.7 Hz, 1H), 1.47 (s, 3H), 1.39 (s, 3H), 1.16-1.01 (m, 1H).

### Example 109

### 3-(Carboxymethoxy)-4-chloro-5-[3-[[(4S)-1-[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (109)

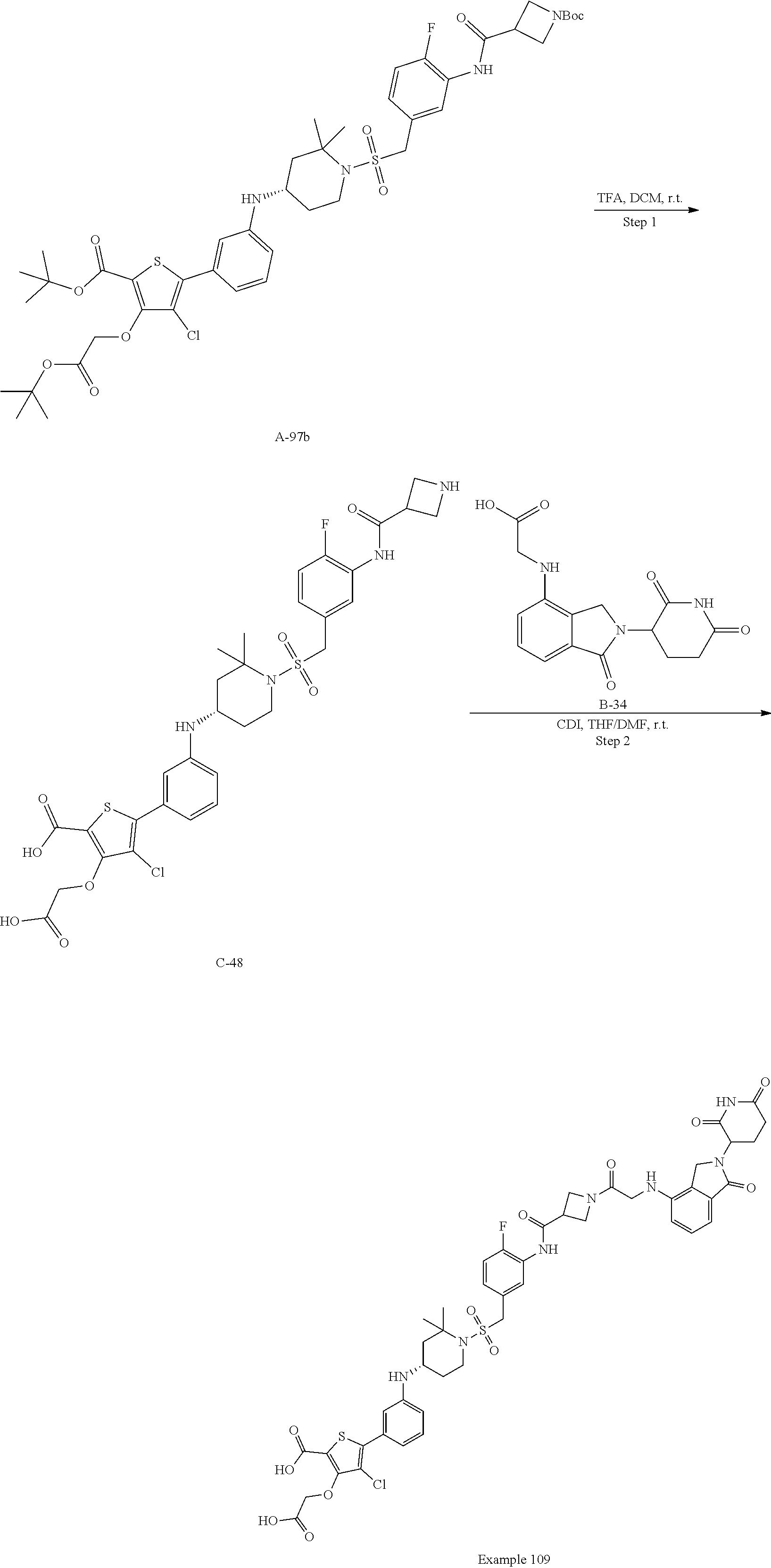

### Step 1:5-[3-[[(4S)-1-[[3-(azetidine-3-carbonylamino)-4-fluoro-phenyl]methylsulfonyl]-2,2-dimethyl-4-piperidyl]amino]phenyl]-3-(carboxymethoxy)-4-chloro-thiophene-2-carboxylic acid (C-48)

5-[3-[[(4S)-1-[[3-(azetidine-3-carbonylamino)-4-fluoro-phenyl]methylsulfonyl]-2,2-dimethyl-4-piperidyl]amino]phenyl]-3-(carboxymethoxy)-4-chloro-thiophene-2-carboxylic acid (C-48, 85 mg, 0.096 mmol, 94% yield) was synthesized from tert-butyl 3-[[5-[[(4S)-4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (A-97b) in a similar fashion to Compound C-1, except using 13 eq. of TFA. LCMS (ESI): m/z 708.7 [M+H]⁺. ¹H NMR (400 MHz, DMSO-d₆) δ 10.03 (s, 1H), 8.73 (s, 2H), 8.09-8.06 (m, 1H), 7.33-7.17 (m, 3H), 6.87-6.80 (m, 2H), 6.70-6.68 (m, 1H), 4.92 (s, 2H), 4.47-4.43 (m, 1H), 4.34-4.31 (m, 1H), 4.13-4.07 (m, 4H), 3.92-3.88 (s, 1H), 3.52-3.56 (m, 4H), 3.14-3.11 (m, 1H), 1.90-1.79 (m, 2H) and 1.54-1.40 (m, 6H).

### Step 2: 3-(carboxymethoxy)-4-chloro-5-[3-[(4S)-1-[[3-[[1-[2-1]2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (109)

3-(carboxymethoxy)-4-chloro-5-[3-[[(4S)-1-[[3-[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (109, 18 mg, 0.015 mmol, 14% yield) was synthesized from 2-[[2-(2,6-dioxo-3-piperidyl)-1-oxo-isoindolin-4-yl]amino]acetic acid (B-34) and 5-[3-[(4S)-1-[[3-(azetidine-3-carbonylamino)-4-fluoro-phenyl]methylsulfonyl]-2,2-dimethyl-4-piperidyl]amino]phenyl]-3-(carboxymethoxy)-4-chloro-thiophene-2-carboxylic acid (C-48) in a similar fashion to Example 23, except using THF:DMF as the solvent. The residue compound was purified by reverse phase HPLC following the method: Column: X-BRIDGE C18 (19×150 mm), 5.0 μm; Mobile phase A: 0.1% TFA in MQ water and B: Acetonitrile; Flow rate: 15 mL/min. LCMS (ESI): m/z 1007.5 [M+H]⁺. ¹H NMR (400 MHz, DMSO-d₆) δ 11.00 (s, 1H), 9.97 (s, 1H), 8.01 (d, J=7.5 Hz, 1H), 7.33-7.25 (m, 2H), 7.23-7.16 (m, 2H), 6.98 (d, J=7.6 Hz, 1H), 6.88 (s, 1H), 6.81 (d, J=7.6 Hz, 1H), 6.71-6.64 (m, 2H), 5.10 (dd, J=13.2, 5.1 Hz, 1H), 4.91 (s, 2H), 4.47-4.38 (m, 2H), 4.37-4.32 (m, 1H), 4.28 (d, J=17.2 Hz, 2H), 4.16 (d, J=17.1 Hz, 2H), 4.04 (dt, J=23.7, 8.8 Hz, 3H), 3.84 (d, J=4.4 Hz, 2H), 2.91 (ddd, J=18.1, 13.6, 5.5 Hz, 2H), 2.68-2.57 (m, 2H), 2.41-2.27 (m, 2H), 2.07-1.96 (m, 1H), 1.89 (d, J=11.9 Hz, 1H), 1.79 (d, J=12.9 Hz, 1H), 1.47 (s, 3H), 1.39 (s, 3H).

### Example 110

### 3-(carboxymethoxy)-4-chloro-5-(3-(((4S)-1-((3-((1R,5S,6r)-3-((2-(2,6-dioxopiperidin-3-yl)-1-oxoisoindolin-4-yl)glycyl)-3-azabicyclo[3.1.0]hexane-6-carboxamido)-4-fluorobenzyl)sulfonyl)-2,2-dimethylpiperidin-4-yl)amino)phenyl)thiophene-2-carboxylic acid (110)

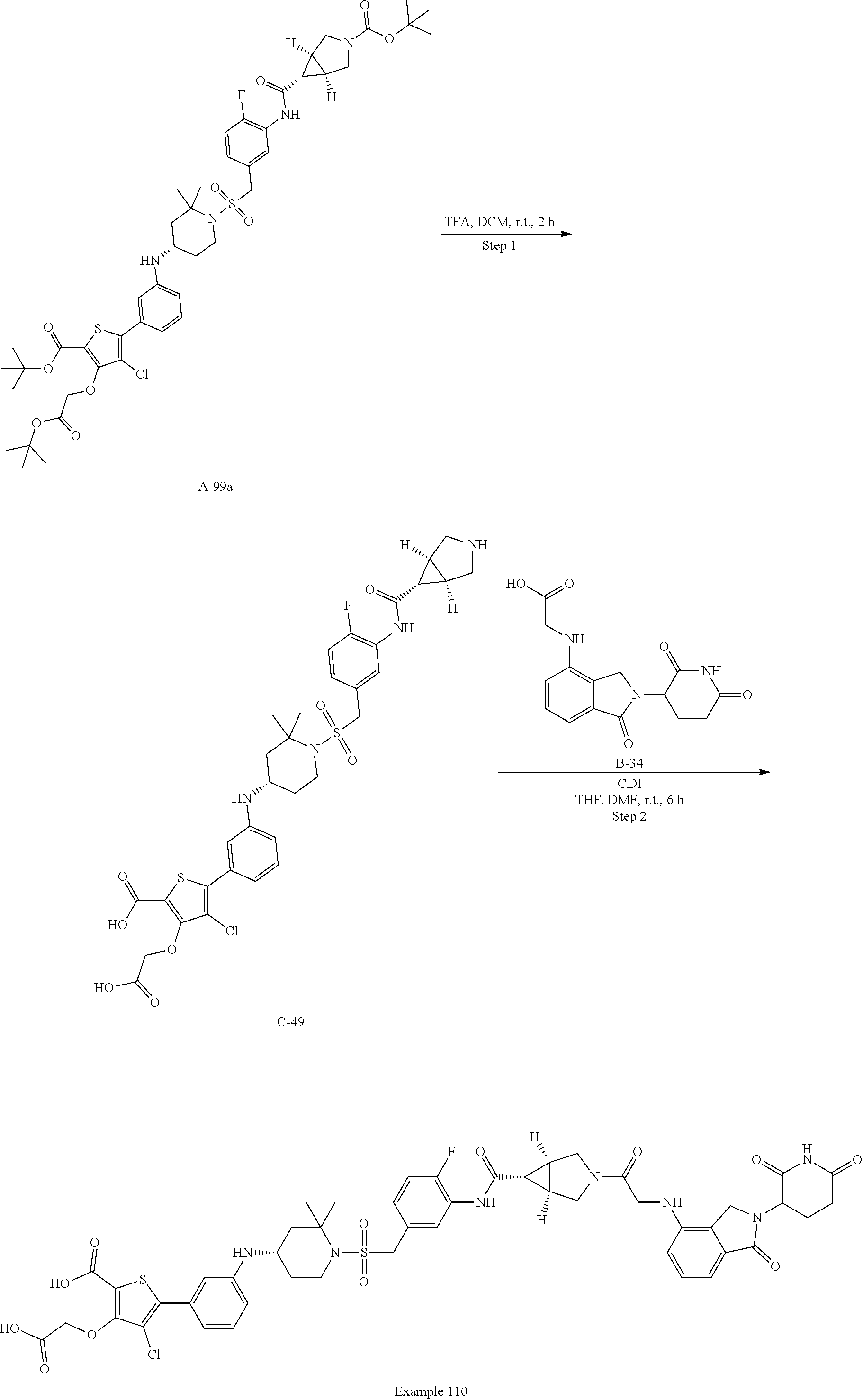

### Step 5-(3-(((S)-1-((3-((1R,5S,6r)-3-azabicyclo[3.1.0]hexane-6-carboxamido)-4-fluorobenzyl)sulfonyl)-2,2-dimethylpiperidin-4-yl)amino)phenyl)-3-(carboxymethoxy)-4-chlorothiophene-2-carboxylic acid (C-49)

5-(3-(((S)-1-((3-((1R,5S,6r)-3-azabicyclo[3.1.0]hexane-6-carboxamido)-4-fluorobenzyl)sulfonyl)-2,2-dimethylpiperidin-4-yl)amino)phenyl)-3-(carboxymethoxy)-4-chlorothiophene-2-carboxylic acid (C-49, 75 mg, 78.43 μmol, 83% yield, TFA salt) was synthesized from tert-butyl (1R,5S,6r)-6-((5-((((S)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99a) in a similar fashion to Compound C-1, except using 72 eq. TFA. LCMS (ES−): m/z 733.1 [M−H]⁻.

### Step 2: 3-(carboxymethoxy)-4-chloro-5-(3-(((4S)-1-((3-((1R,5S,6r)-3-((2-(2,6-dioxopiperidin-3-yl)-1-oxoisoindolin-4-yl)glycyl)-3-azabicyclo[3.1.0]hexane-6-carboxamido)-4-fluorobenzyl)sulfonyl)-2,2-dimethylpiperidin-4-yl)amino)phenyl)thiophene-2-carboxylic acid (110)

3-(carboxymethoxy)-4-chloro-5-(3-(((4S)-1-((3-((1R,5S,6r)-3-((2-(2,6-dioxopiperidin-3-yl)-1-oxoisoindolin-4-yl)glycyl)-3-azabicyclo[3.1.0]hexane-6-carboxamido)-4-fluorobenzyl)sulfonyl)-2,2-dimethylpiperidin-4-yl)amino)phenyl)thiophene-2-carboxylic acid (110, 25 mg, 20.92 μmol, 33% yield, TFA salt) was synthesized from 2-[[2-(2,6-dioxo-3-piperidyl)-1-oxo-isoindolin-4-yl]amino]acetic acid (B-34) and 5-(3-(((S)-1-((3-((1R,5S,6r)-3-azabicyclo[3.1.0]hexane-6-carboxamido)-4-fluorobenzyl)sulfonyl)-2,2-dimethylpiperidin-4-yl)amino)phenyl)-3-(carboxymethoxy)-4-chlorothiophene-2-carboxylic acid (C-49) in a similar fashion to Example 23, except using THF:DMF as a solvent. The residue was purified by reverse phase prep HPLC [Purification method: Column: XSELECT C18 (150×19 mm) 5 μm, mobile phase: 0.1% TFA in water and MeCN]. LCMS (ES+): m/z 1034.1 [M+H]⁺. ¹H NMR (400 MHz, DMSO-d₆) δ 11.01 (s, 1H), 10.01 (s, 1H), 8.02 (d, J=7.4 Hz, 1H), 7.33-7.13 (m, 4H), 6.99 (d, J=7.4 Hz, 1H), 6.88 (s, 1H), 6.84 (d, J=7.7 Hz, 1H), 6.72 (t, J=8.1 Hz, 2H), 5.12 (dd, J=13.2, 5.1 Hz, 1H), 4.91 (s, 2H), 4.41 (d, J=13.9 Hz, 1H), 4.34-4.25 (m, 2H), 4.18 (d, J=17.0 Hz, 1H), 4.04-3.84 (m, 4H), 3.45-3.37 (m, 2H), 3.11 (t, J=12.4 Hz, 1H), 2.98-2.84 (m, 1H), 2.69-2.57 (m, 1H), 2.42-2.29 (m, 3H), 2.21-2.13 (m, 1H), 2.08-1.97 (m, 2H), 1.94-1.83 (m, 2H), 1.77 (d, J=12.9 Hz, 1H), 1.44 (s, 3H), 1.37 (s, 3H), 1.14-1.03 (m, 1H).

### Example 111

### 3-(carboxymethoxy)-4-chloro-5-(3-(((4R)-1-((3-((1R,5S,6r)-3-((2-(2,6-dioxopiperidin-3-yl)-1-oxoisoindolin-4-yl)glycyl)-3-azabicyclo[3.1.0]hexane-6-carboxamido)-4-fluorobenzyl)sulfonyl)-2,2-dimethylpiperidin-4-yl)amino)phenyl)thiophene-2-carboxylic acid (111)

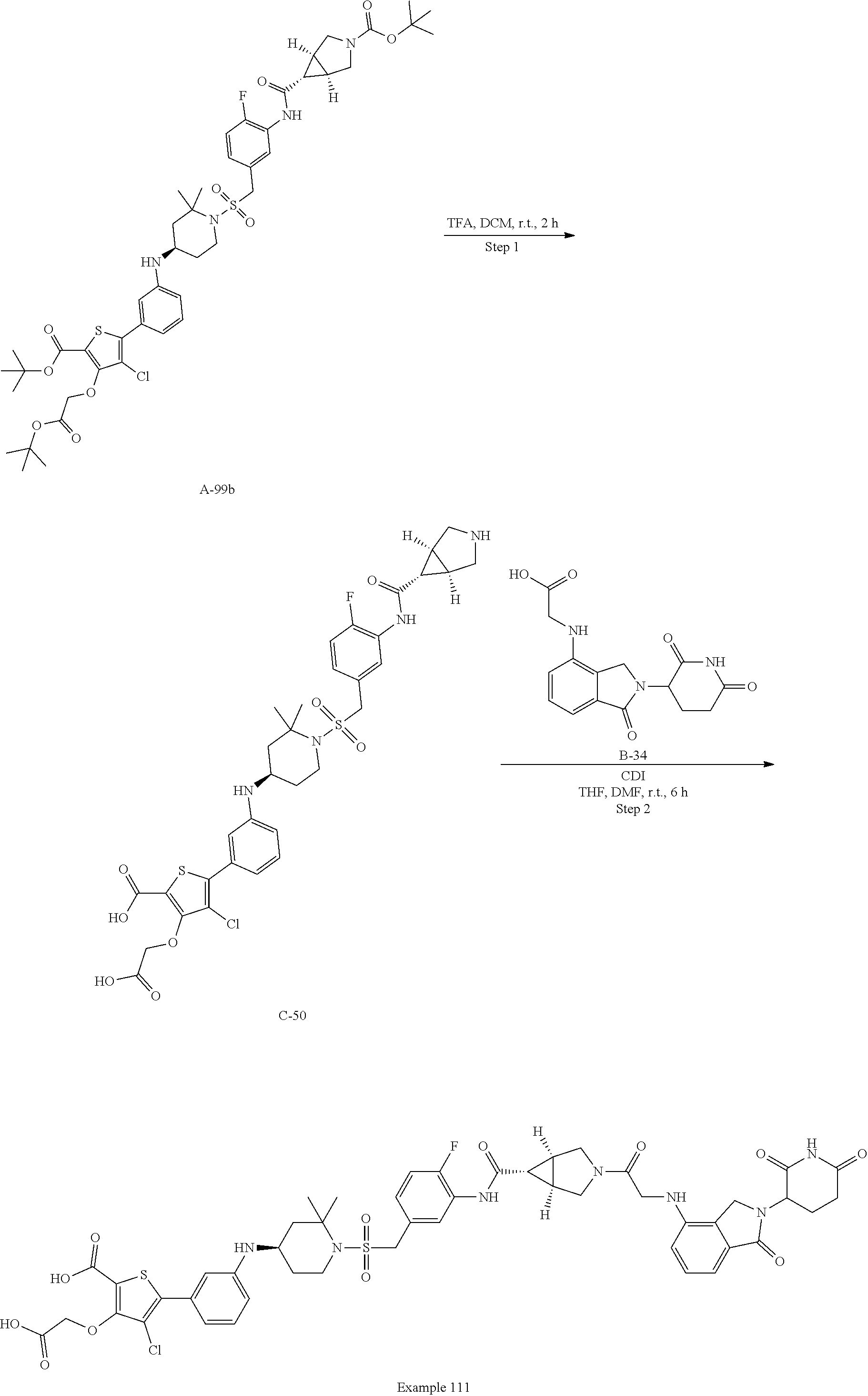

3-(carboxymethoxy)-4-chloro-5-(3-(((4R)-1-((3-((1R,5S,6r)-3-((2-(2,6-dioxopiperidin-3-yl)-1-oxoisoindolin-4-yl)glycyl)-3-azabicyclo[3.1.0]hexane-6-carboxamido)-4-fluorobenzyl)sulfonyl)-2,2-dimethylpiperidin-4-yl)amino)phenyl)thiophene-2-carboxylic acid (111, 17 mg, 13.63 μmol, 22% yield, TFA salt) was synthesized from tert-butyl (1R,5S,6r)-6-((5-((((R)-4-((3-(4-(2-(tert-butoxy)-2-oxoethoxy)-5-(tert-butoxycarbonyl)-3-chlorothiophen-2-yl)phenyl)amino)-2,2-dimethylpiperidin-1-yl)sulfonyl)methyl)-2-fluorophenyl)carbamoyl)-3-azabicyclo[3.1.0]hexane-3-carboxylate (A-99b) over two steps following the same procedure used for the synthesis of Example 110. LCMS (ES+): m/z 1034.1 [M+H]⁺. ¹H NMR (400 MHz, DMSO-d₆) δ 11.01 (s, 1H), 10.02 (s, 1H), 8.01 (d, J=7.5 Hz, 1H), 7.32-7.14 (m, 4H), 6.99 (d, J=7.7 Hz, 1H), 6.88-6.85 (m, 1H), 6.82 (d, J=7.7 Hz, 1H), 6.75-6.67 (m, 2H), 5.11 (dd, J=13.2, 5.2 Hz, 1H), 4.91 (s, 2H), 4.41 (d, J=13.9 Hz, 1H), 4.33-4.26 (m, 2H), 4.18 (d, J=17.1 Hz, 1H), 4.00 (dd, J=17.1, 4.2 Hz, 1H), 3.86 (s, 2H), 3.15-3.06 (m, 2H), 2.98-2.86 (m, 2H), 2.66-2.57 (m, 1H), 2.43-2.29 (m, 3H), 2.21-2.15 (m, 1H), 2.08-1.98 (m, 2H), 1.93-1.82 (m, 2H), 1.76 (d, J=12.5 Hz, 1H), 1.44 (s, 3H), 1.37 (s, 3H), 1.16-0.99 (m, 1H).

### Example 112

### 3-(carboxymethoxy)-4-chloro-5-[3-[[1-[[3-[[1-[1-(2,6-dioxo-3-piperidyl)-3-methyl-2-oxo-benzimidazol-5-yl]piperidine-4-carbonyl]amino]phenyl]methylsulfonyl]-4-piperidyl]amino]phenyl]thiophene-2-carboxylic acid (112)

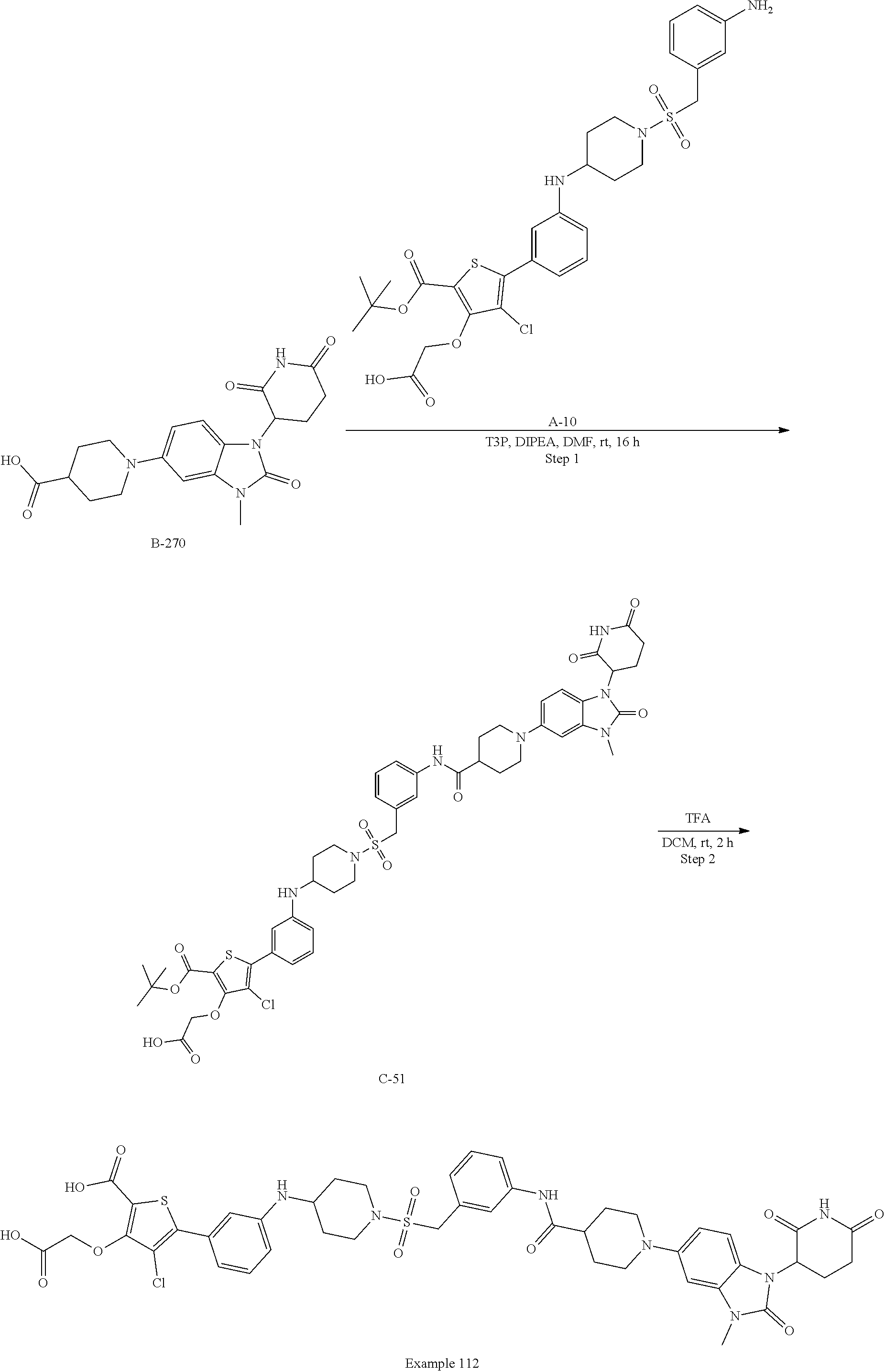

### Step 1: 2-[[2-tert-butoxycarbonyl-4-chloro-5-[3-[[1-[[3-[[1-[1-(2,6-dioxo-3-piperidyl)-3-methyl-2-oxo-benzimidazol-5-yl]piperidine-4-carbonyl]amino]phenyl]methylsulfonyl]-4-piperidyl]amino]phenyl]-3-thienyl]oxy]acetic acid (C-51)

2-[[2-tert-butoxycarbonyl-4-chloro-5-[3-[[1-[[3-[[1-[1-(2,6-dioxo-3-piperidyl)-3-methyl-2-oxo-benzimidazol-5-yl]piperidine-4-carbonyl]amino]phenyl]methylsulfonyl]-4-piperidyl]amino]phenyl]-3-thienyl]oxy]acetic acid (C-51, 15 mg, 13.71 μmol, 6% yield, formate salt) was synthesized from 1-[1-(2,6-dioxo-3-piperidyl)-3-methyl-2-oxo-benzimidazol-5-yl]piperidine-4-carboxylic acid (B-270) and 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) in a similar fashion to Compound C-6, except using DIPEA as the base. The residue was purified by reverse phase prep HPLC (Column: ATLANTIS-C18, 19×150 mm, 5 μm; Mobile phase A: 0.1% formic acid in water; Mobile phase B: MeCN). LCMS (ES+): m/z 1005.1 [M+H]⁺.

### Step 2: 3-(carboxymethoxy)-4-chloro-5-[3-[[1-[[3-[1-[1-(2,6-dioxo-3-piperidyl)-3-methyl-2-oxo-benzimidazol-S-yl]piperidine-4-carbonyl]amino]phenyl]methylsulfonyl]-4-piperidyl]amino]phenyl]thiophene-2-carboxylic acid (112)

3-(carboxymethoxy)-4-chloro-5-[3-[[1-[[3-[[1-[1-(2,6-dioxo-3-piperidyl)-3-methyl-2-oxo-benzimidazol-5-yl]piperidine-4-carbonyl]amino]phenyl]methylsulfonyl]-4-piperidyl]amino]phenyl]thiophene-2-carboxylic acid (112, 5 mg, 4.59 μmol, 36% yield, TFA salt) was synthesized from 2-[[2-tert-butoxycarbonyl-4-chloro-5-[3-[[1-[[3-[[1-[1-(2,6-dioxo-3-piperidyl)-3-methyl-2-oxo-benzimidazol-5-yl]piperidine-4-carbonyl]amino]phenyl]methylsulfonyl]-4-piperidyl]amino]phenyl]-3-thienyl]oxy]acetic acid (C-51) in a similar fashion to Compound C-1, except using 5 eq. of TFA. The residue was triturated with diethyl ether. LCMS (ES+): m/z 948.7 [M+H]⁺. ¹H NMR (400 MHz, DMSO-d₆) δ 10.19 (s, 1H), 9.18 (s, 1H), 6.83 (s, 1H), 6.68 (d, J=8.2 Hz, 1H), 6.42 (t, J=7.9 Hz, 1H), 6.34-6.26 (m, 1H), 6.22-6.17 (m, 1H), 6.15-6.05 (m, 2H), 5.99-5.95 (m, 1H), 5.91 (d, J=7.5 Hz, 1H), 5.84-5.77 (m, 1H), 4.41 (dd, J=12.9, 5.4 Hz, 1H), 4.01 (s, 2H), 3.47 (s, 2H), 2.81-2.73 (m, 4H), 2.07 (t, J=11.5 Hz, 4H), 2.01-1.93 (m, 3H), 1.85-1.69 (m, 5H), 1.14-0.93 (m, 7H), 0.53-0.40 (m, 2H).

### Example 113

### 3-(carboxymethoxy)-4-chloro-5-[3-[[1-[[3-[[2-[1-(2,6-dioxo-3-piperidyl)-3-methyl-2-oxo-benzimidazol-5-yl]-2,7-diazaspiro[3.5]nonane-7-carbonyl]amino]phenyl]methylsulfonyl]-4-piperidyl]amino]phenyl]thiophene-2-carboxylic acid (113)

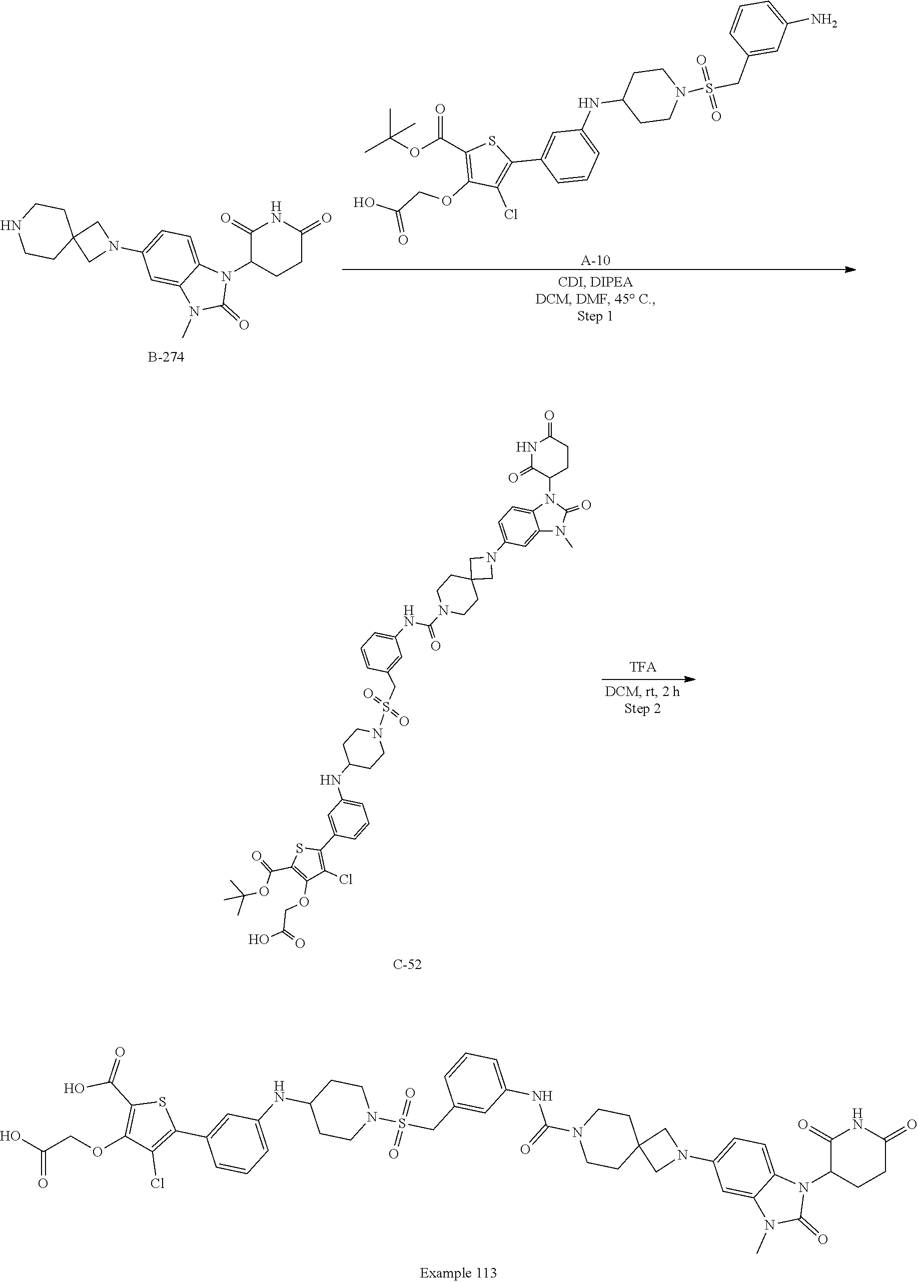

### Step 1: 2-[2-tert-butoxycarbonyl-4-chloro-5-[3-[[1-[[3-[[2-[1-(2,6-dioxo-3-piperidyl)-3-methyl-2-oxo-benzimidazol-5-yl]-2,7-diazaspiro[3.5]nonane-7-carbonyl]amino]phenyl]methylsulfonyl]-4-piperidyl]amino]phenyl]-3-thienyl]oxy]acetic acid (C-52)

2-[[2-tert-butoxycarbonyl-4-chloro-5-[3-[[1-[[3-[[2-[1-(2,6-dioxo-3-piperidyl)-3-methyl-2-oxo-benzimidazol-5-yl]-2,7-diazaspiro[3.5]nonane-7-carbonyl]amino]phenyl]methylsulfonyl]-4-piperidyl]amino]phenyl]-3-thienyl]oxy]acetic acid (C-52, 28 mg, 19.24 μmol, 9% yield, formic acid salt) was synthesized from 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) and 3-[5-(2,7-diazaspiro[3.5]nonan-2-yl)-3-methyl-2-oxo-benzimidazol-1-yl]piperidine-2,6-dione (B-274) in a similar fashion to Example 23, except using excess DIPEA as base, DMF:DCM as solvent and heating the reaction to 45° C. The residue was purified by reverse phase prep HPLC [Purification method: Column: X-Bridge C18 (150×19) mm, 5 μm; 0.1% formic acid in MQ water and MeCN]. LCMS (ES+): m/z 1045.2 [M+H]⁺.

### Step 2: 3-(carboxymethoxy)-4-chloro-5-[3-[[1-[[3-[[2-[1-(2,6-dioxo-3-piperidyl)-3-methyl-2-oxo-benzimidazol-S-yl]-2,7-diazaspiro[3.5]nonane-7-carbonyl]amino]phenyl]methylsulfonyl]-4-piperidyl]amino]phenyl]thiophene-2-carboxylic acid (113)

3-(carboxymethoxy)-4-chloro-5-[3-[[1-[[3-[[2-[1-(2,6-dioxo-3-piperidyl)-3-methyl-2-oxo-benzimidazol-5-yl]-2,7-diazaspiro[3.5]nonane-7-carbonyl]amino]phenyl]methylsulfonyl]-4-piperidyl]amino]phenyl]thiophene-2-carboxylic acid (113, 20 mg, 16.80 μmol, 63% yield, TFA salt) was synthesized from 2-[[2-tert-butoxycarbonyl-4-chloro-5-[3-[[1-[[3-[2-[1-(2,6-dioxo-3-piperidyl)-3-methyl-2-oxo-benzimidazol-5-yl]-2,7-diazaspiro[3.5]nonane-7-carbonyl]amino]phenyl]methylsulfonyl]-4-piperidyl]amino]phenyl]-3-thienyl]oxy]acetic acid (C-52) in a similar fashion to Compound C-1. The residue was triturated with diethyl ether. LCMS (ES+): m/z 989.2 [M+H]⁺. ¹H NMR (400 MHz, DMSO-d6): δ 13.5 (bs, 1H), 11.06 (s, 1H), 8.64 (s, 1H), 7.58 (s, 1H), 7.40 (d, J=8.00 Hz, 1H), 7.27-7.19 (m, 2H), 6.92 (d, J=8.40 Hz, 1H), 6.86 (s, 1H), 6.81 (d, J=7.60 Hz, 1H), 6.70 (d, J=8.40 Hz, 1H), 6.32 (d, J=1.60 Hz, 1H), 6.12 (dd, J=1.60, 8.40 Hz, 1H), 5.88 (d, J=6.00 Hz, 1H), 5.28 (dd, J=5.20, 12.40 Hz, 1H), 4.90 (s, 2H), 4.35 (s, 2H), 3.58-3.29 (m, 12H), 3.01-2.90 (m, 3H), 2.68-2.67 (m, 1H), 1.99-1.92 (m, 3H), 1.75 (m, 4H), 1.37-1.35 (m, 2H).

### Example 114

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (114)

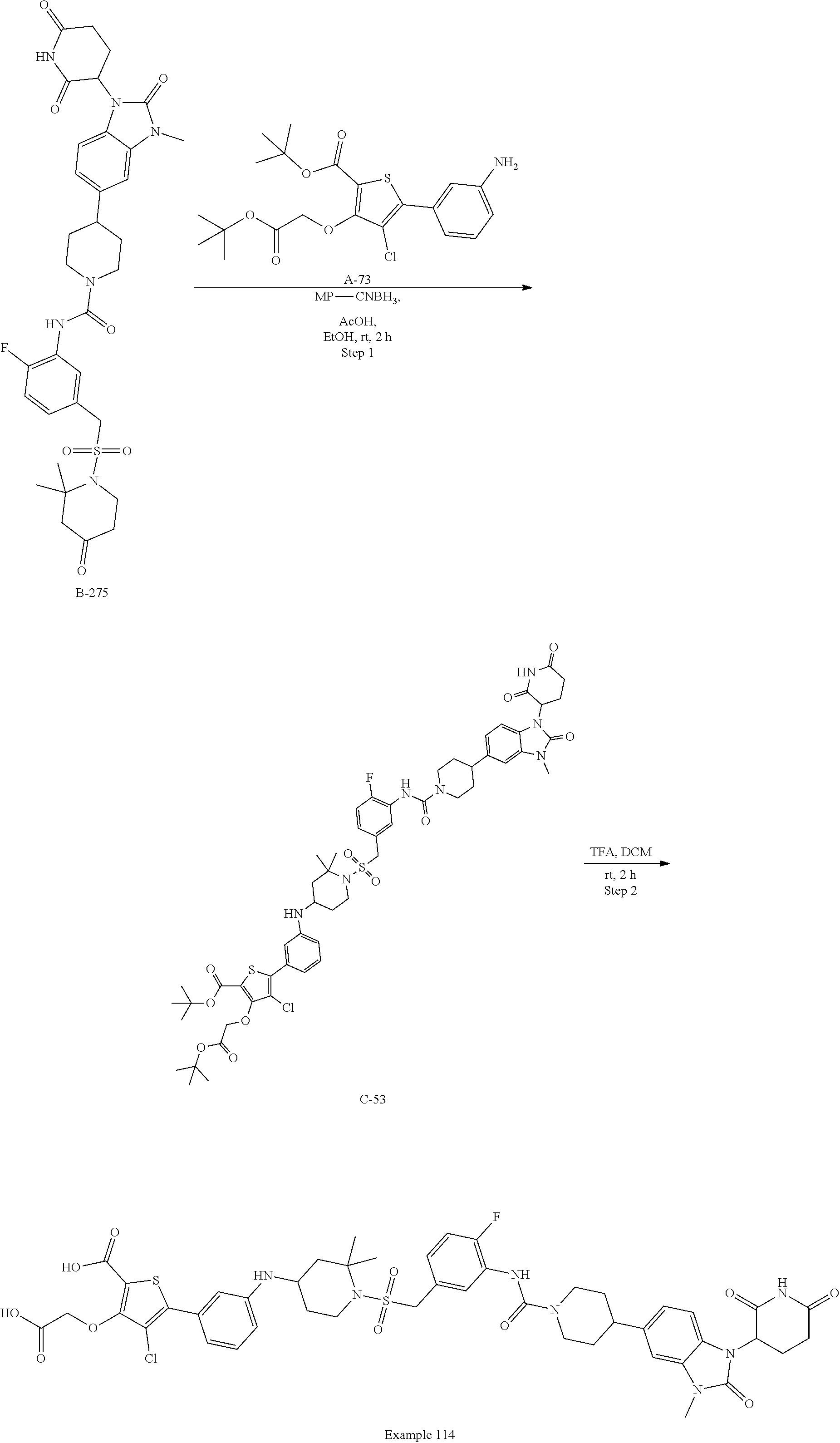

### Step 1; tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylate (C-53)

Tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylate (C-53, 300 mg, 114.12 μmol, 46% yield) was synthesized from N-[5-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-275) and tert-butyl 5-(3-aminophenyl)-3-(2-tert-butoxy-2-oxo-ethoxy)-4-chloro-thiophene-2-carboxylate (A-73) in a similar fashion to Compound C-38. The residue was triturated with diethyl ether. LCMS (ES+): m/z 1106.2 [M+H]⁺.

### Step 2: 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (114)

3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (114, 30 mg, 26.34 μmol, 10% yield, TFA salt) was synthesized from tert-butyl 3-(2-tert-butoxy-2-oxo-ethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylate (C-53) in a similar fashion to Compound C-1, except using 24 eq. TFA. The residue was purified by reverse phase prep HPLC [Purification method: Column: XSelect C18 (19×150 mm) 5.0 μm, Mobile phase A: 0.1% TFA in water and mobile phase B: Acetonitrile]. LCMS (ES+): m/z 994.2 [M+H]⁺. ¹H NMR (400 MHz, DMSO-d₆) δ 11.09 (s, 1H), 8.37 (s, 1H), 7.51 (dd, J=7.7, 2.1 Hz, 1H), 7.24-7.13 (m, 3H), 7.12-7.10 (m, 1H), 7.02 (d, J=8.1 Hz, 1H), 6.94-6.90 (m, 1H), 6.88-6.85 (m, 1H), 6.83-6.78 (m, 1H), 6.69 (dd, J=8.5, 2.3 Hz, 1H), 5.67 (s, 1H), 5.34 (dd, J=12.8, 5.4 Hz, 1H), 4.90 (s, 2H), 4.43 (d, J=13.9 Hz, 1H), 4.36-4.20 (m, 3H), 3.33 (s, 3H), 3.11 (t, J=12.7 Hz, 2H), 2.98-2.84 (m, 3H), 2.82-2.70 (m, 1H), 2.66-2.58 (m, 2H), 2.05-1.95 (m, 1H), 1.89-1.74 (m, 4H), 1.72-1.57 (m, 2H), 1.50 (s, 3H), 1.46-1.35 (m, 4H), 1.09-0.93 (m, 1H).

### Example 115

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (115)

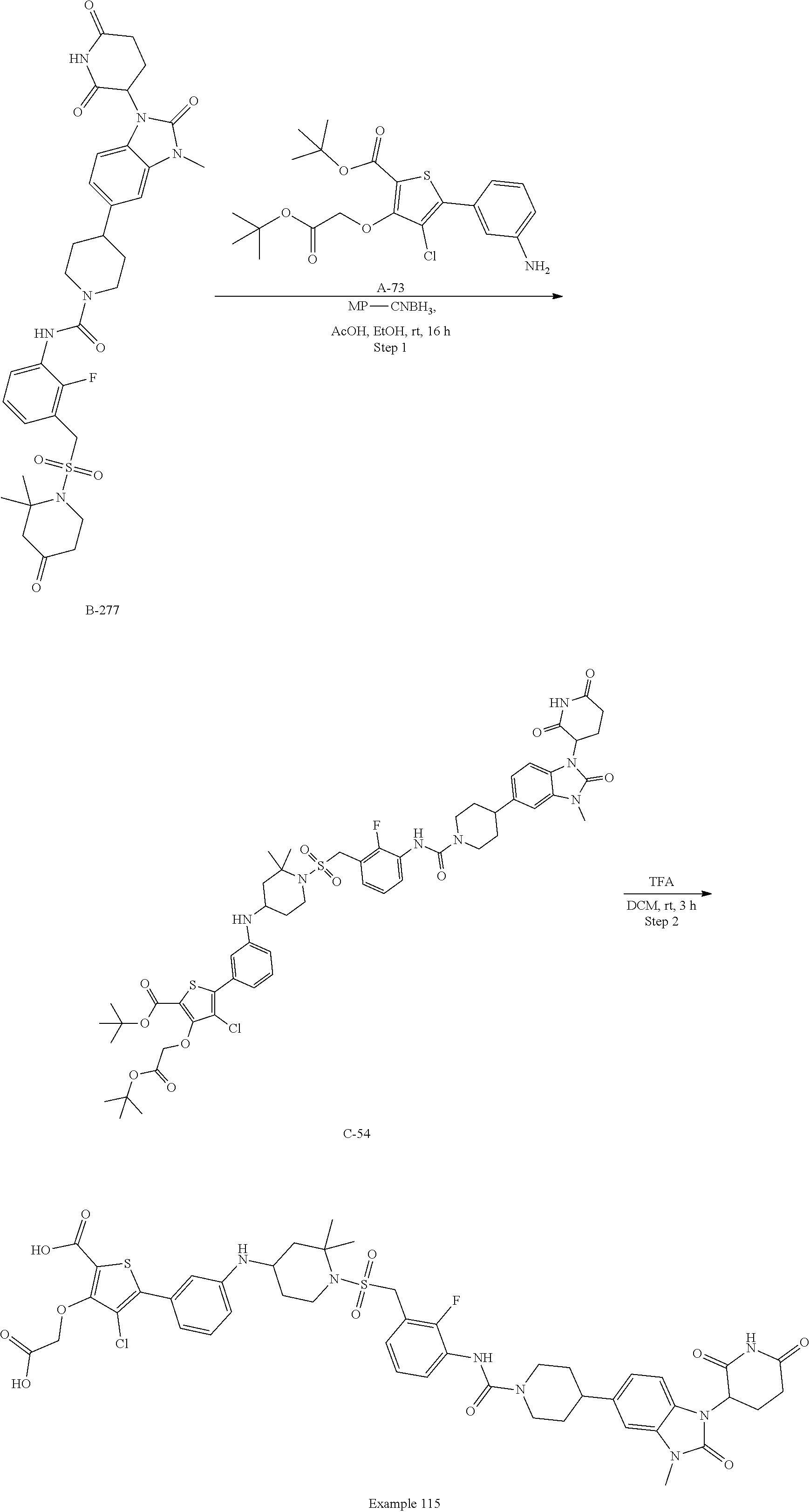

### Step 1: tert-butyl 3-(2-(tert-butoxy)-2-oxoethoxy)-4-chloro-5-(3-((1-((3-(4-(1-(2,6-dioxopiperidin-3-yl)-3-methyl-2-oxo-2,3-dihydro-1H-benzo[d]imidazol-5-yl)piperidine-1-carboxamido)-2-fluorobenzyl)sulfonyl)-2,2-dimethylpiperidin-4-yl)amino)phenyl)thiophene-2-carboxylate (C-54)

Tert-butyl 3-(2-(tert-butoxy)-2-oxoethoxy)-4-chloro-5-(3-((1-((3-(4-(1-(2,6-dioxopiperidin-3-yl)-3-methyl-2-oxo-2,3-dihydro-1H-benzo[d]imidazol-5-yl)piperidine-1-carboxamido)-2-fluorobenzyl)sulfonyl)-2,2-dimethylpiperidin-4-yl)amino)phenyl)thiophene-2-carboxylate (C-54, 28 mg, 15.02 μmol, 41% yield) was synthesized from N-[3-[(2,2-dimethyl-4-oxo-1-piperidyl)sulfonylmethyl]-2-fluoro-phenyl]-4-[1-(2,6-dioxo-3-piperidyl)-3-methyl-2-oxo-benzimidazol-5-yl]piperidine-1-carboxamide (B-277) and tert-butyl 5-(3-aminophenyl)-3-(2-tert-butoxy-2-oxo-ethoxy)-4-chloro-thiophene-2-carboxylate (A-73) in a similar fashion to Compound C-38. LCMS (ES+): m/z 1107.2 [M+Na]⁺.

### Step 2: 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (115)

3-(carboxymethoxy)-4-chloro-5-[3-[[1-[[3-[[4-[[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (115, 15 mg, 13.18 μmol, 52% yield) was synthesized from tert-butyl 3-(2-(tert-butoxy)-2-oxoethoxy)-4-chloro-5-(3-((1-((3-(4-(1-(2,6-dioxopiperidin-3-yl)-3-methyl-2-oxo-2,3-dihydro-1H-benzo[d]imidazol-5-yl)piperidine-1-carboxamido)-2-fluorobenzyl)sulfonyl)-2,2-dimethylpiperidin-4-yl)amino)phenyl)thiophene-2-carboxylate (C-54) in a similar fashion to Compound C-1, except using 156 eq. of TFA. The residue was purified by reverse phase prep HPLC [Purification method: Column: X-BRIDGE C18 (19×150 mm) 5.0 μm; Solvent A: 0.1% TFA in water; Solvent B: Acetonitrile]. LCMS (ES+): m/z 994.7 [M+H]⁺. ¹H NMR (400 MHz, DMSO-d₆) δ 11.09 (s, 1H), 8.40 (s, 1H), 7.46-7.39 (m, 1H), 7.24-7.08 (m, 4H), 7.01 (d, J=8.1 Hz, 1H), 6.92 (d, J=8.3 Hz, 1H), 6.88-6.85 (m, 1H), 6.80 (d, J=7.6 Hz, 1H), 6.71-6.66 (m, 1H), 5.33 (dd, J=12.8, 5.4 Hz, 1H), 4.88 (s, 2H), 4.47 (d, J=13.9 Hz, 1H), 4.38 (d, J=14.0 Hz, 1H), 4.27 (d, J=12.8 Hz, 2H), 3.33 (s, 3H), 3.18-3.09 (m, 2H), 2.98-2.84 (m, 4H), 2.83-2.70 (m, 2H), 2.66-2.58 (m, 2H), 2.04-1.95 (m, 1H), 1.86-1.73 (m, 4H), 1.72-1.57 (m, 2H), 1.52 (s, 3H), 1.49-1.42 (m, 1H), 1.39 (s, 3H), 1.11-0.96 (m, 1H).

### Example 116

### 3-(carboxymethoxy)-4-chloro-5-[3-[[(4S)-1-[3-[3-[[1-(2,6-dioxo-3-piperidyl)-3-methyl-2-oxo-benzimidazol-5-yl]oxyazetidine-1-carbonyl]amino]phenyl]methylsulfonyl]-2,2-dimethyl-4-piperidyl]amino]phenyl]thiophene-2-carboxylic acid (116)

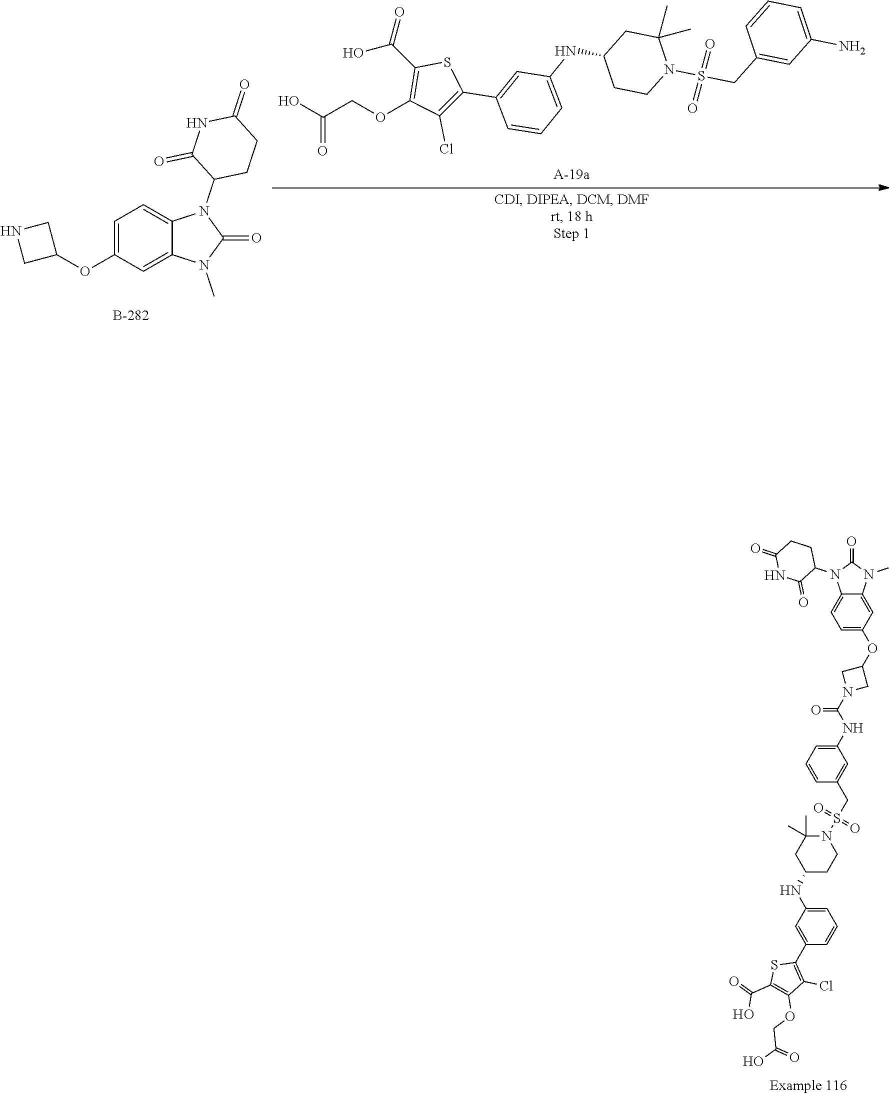 [SMILES: Cn1c(=O)n(C2CCC(=O)NC2=O)c2ccc(OC3CNC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(OC3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21]

### Step 1: 3-(carboxymethoxy)-4-chloro-5-[3-[[(4S)-1-[[3-[3-[[1-(2,6-dioxo-3-piperidyl)-3-methyl-2-oxo-benzimidazol-5-yl]oxyazetidine-1-carbonyl]amino]phenyl]methylsulfonyl]-2,2-dimethyl-4-piperidyl]amino]phenyl]thiophene-2-carboxylic acid (116)

3-(carboxymethoxy)-4-chloro-5-[3-[[(4S)-1-[3-[3-[[1-(2,6-dioxo-3-piperidyl)-3-methyl-2-oxo-benzimidazol-5-yl]oxyazetidine-1-carbonyl]amino]phenyl]methylsulfonyl]-2,2-dimethyl-4-piperidyl]amino]phenyl]thiophene-2-carboxylic acid (116, 6.5 mg, 5.99 μmol, 9% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(azetidin-3-yloxy)-3-methyl-2-oxo-benzimidazol-1-yl]piperidine-2,6-dione (B-282) in a similar fashion to Example 23, except using DCM:DMF as the solvent. The residue was purified by reverse phase prep HPLC [Purification method: Column: X-bridge, C18 (150×19 mm), 5 μm; Mobile phase A: 0.1% TFA in water and Mobile phase B: MeCN]. LCMS (ES+): m/z 964.5 [M+H]⁺. ¹H NMR (400 MHz, DMSO-d₆) δ 11.09 (s, 1H), 8.66 (s, 1H), 7.58-7.55 (m, 1H), 7.53-7.48 (m, 1H), 7.24 (t, J=7.9 Hz, 1H), 7.19 (t, J=7.9 Hz, 1H), 7.02 (d, J=8.6 Hz, 1H), 6.98 (d, J=7.7 Hz, 1H), 6.88-6.85 (m, 1H), 6.82-6.78 (m, 2H), 6.68 (dd, J=8.1, 2.2 Hz, 1H), 6.54 (dd, J=8.5, 2.4 Hz, 1H), 5.32 (dd, J=12.8, 5.4 Hz, 1H), 5.09-4.99 (m, 1H), 4.90 (s, 2H), 4.47-4.39 (m, 2H), 4.35 (d, J=13.8 Hz, 1H), 4.24 (d, J=13.7 Hz, 1H), 3.92 (dd, J=9.5, 3.8 Hz, 2H), 3.31 (s, 3H), 3.16-3.07 (m, 2H), 2.95-2.83 (m, 2H), 2.75-2.55 (m, 4H), 2.05-1.95 (m, 1H), 1.89-1.81 (m, 1H), 1.81-1.73 (m, 1H), 1.46 (s, 3H), 1.44-1.40 (m, 1H), 1.38 (s, 3H), 1.17-1.03 (m, 1H).

### Example 117

### 3-(carboxymethoxy)-4-chloro-5-[3-[[(4S)-1-[[3-[[1-[1-(2,6-dioxo-3-piperidyl)-3-methyl-2-oxo-benzimidazol-5-yl]-4-piperidyl]oxycarbonylamino]phenyl]methylsulfonyl]-2,2-dimethyl-4-piperidyl]amino]phenyl]thiophene-2-carboxylic acid (117)

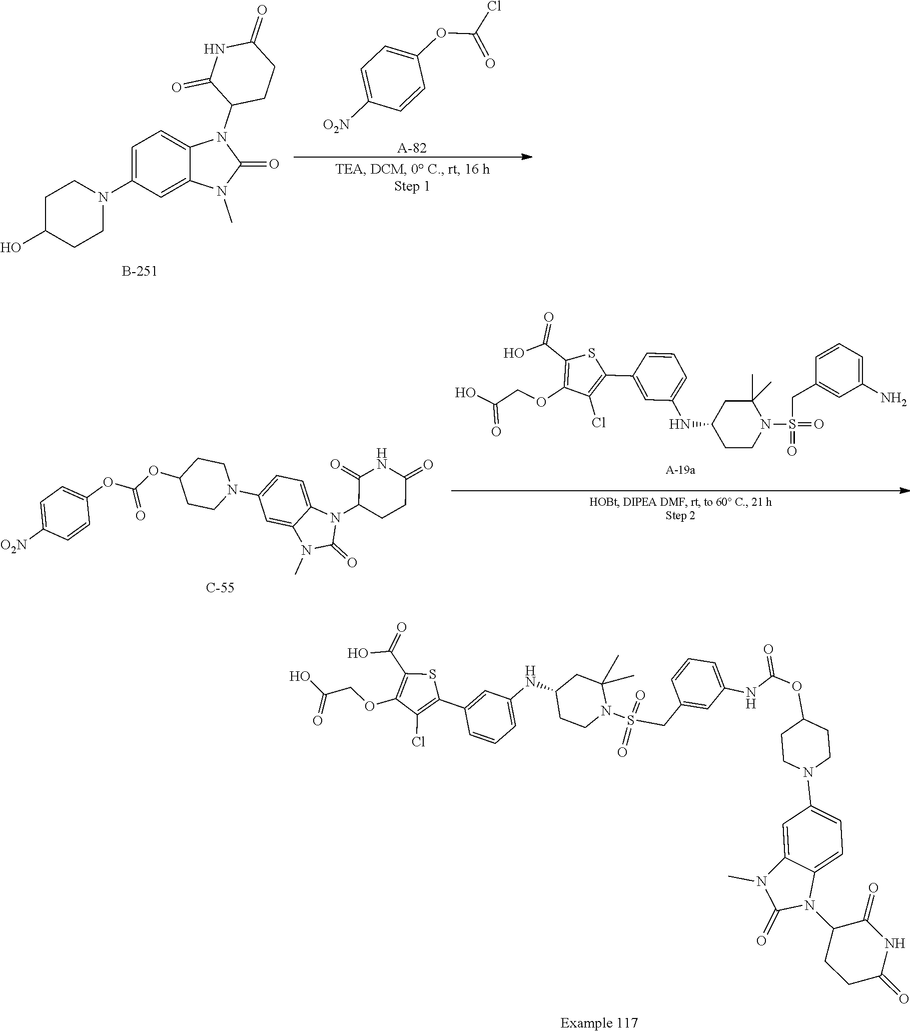

### Step 1: [1-[1-(2,6-dioxo-3-piperidyl)-3-methyl-2-oxo-benzimidazol-5-yl]-4-piperidyl](4-nitrophenyl)carbonate (C-55)

[1-[1-(2,6-dioxo-3-piperidyl)-3-methyl-2-oxo-benzimidazol-5-yl]-4-piperidyl](4-nitrophenyl)carbonate (C-55, 70 mg, 41.45 μmol, 30% yield) was synthesized from 3-[5-(4-hydroxy-1-piperidyl)-3-methyl-2-oxo-benzimidazol-1-yl]piperidine-2,6-dione (B-251) in a similar fashion to Compound C-42. LCMS (ES+): m/z 523.9 [M+H]⁺.

### Step 2: 3-(carboxymethoxy)-4-chloro-5-[3-[[(4S)-1-[[3-[[1-[1-(2,6-dioxo-3-piperidyl)-3-methyl-2-oxo-benzimidazol-5-yl]-4-piperidyl]oxycarbonylamino]phenyl]methylsulfonyl]-2,2-dimethyl-4-piperidyl]amino]phenyl]thiophene-2-carboxylic acid (117)

3-(carboxymethoxy)-4-chloro-5-[3-[[(4S)-1-[[3-[[1-[1-(2,6-dioxo-3-piperidyl)-3-methyl-2-oxo-benzimidazol-5-yl]-4-piperidyl]oxycarbonylamino]phenyl]methylsulfonyl]-2,2-dimethyl-4-piperidyl]amino]phenyl]thiophene-2-carboxylic acid (117, 10 mg, 8.42 μmol, 20% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 1-[1-(2,6-dioxo-3-piperidyl)-3-methyl-2-oxo-benzimidazol-5-yl]-4-piperidyl](4-nitrophenyl)carbonate (C-55) in a similar fashion to Example 85, except heating at 60° C. The residue was purified by reverse phase prep HPLC (Column: XSelect C18 (150×19) mm, 5 μm; Mobile phase A: 0.1% TFA in water; Mobile phase B: MeCN). LCMS (ES+): m/z 992.3 [M+H]⁺. ¹H NMR (400 MHz, DMSO-d₆) δ 11.09 (s, 1H), 9.76 (s, 1H), 7.60 (s, 1H), 7.44 (d, J=8.2 Hz, 1H), 7.29 (t, J=7.9 Hz, 1H), 7.25-7.16 (m, 1H), 7.12-7.02 (m, 3H), 6.89-6.83 (m, 2H), 6.80 (d, J=7.6 Hz, 1H), 6.71-6.66 (m, 1H), 5.32 (dd, J=12.9, 5.4 Hz, 1H), 4.94-4.82 (m, 3H), 4.38 (d, J=13.8 Hz, 1H), 4.26 (s, 1H), 3.33 (s, 3H), 2.96-2.83 (m, 2H), 2.75-2.58 (m, 3H), 2.18-2.06 (m, 2H), 2.05-1.96 (m, 1H), 1.92-1.74 (m, 4H), 1.45 (s, 3H), 1.44-1.35 (m, 4H), 1.16-1.03 (m, 1H).

### Example 118

### 3-(carboxymethoxy)-4-chloro-5-[3-[[(4S)-1-[3-[4-[1-(2,6-dioxo-3-piperidyl)-3-methyl-2-oxo-benzimidazol-5-yl]oxypiperidine-1-carbonyl]amino]phenyl]methylsulfonyl]-2,2-dimethyl-4-piperidyl]amino]phenyl]thiophene-2-carboxylic acid (118)

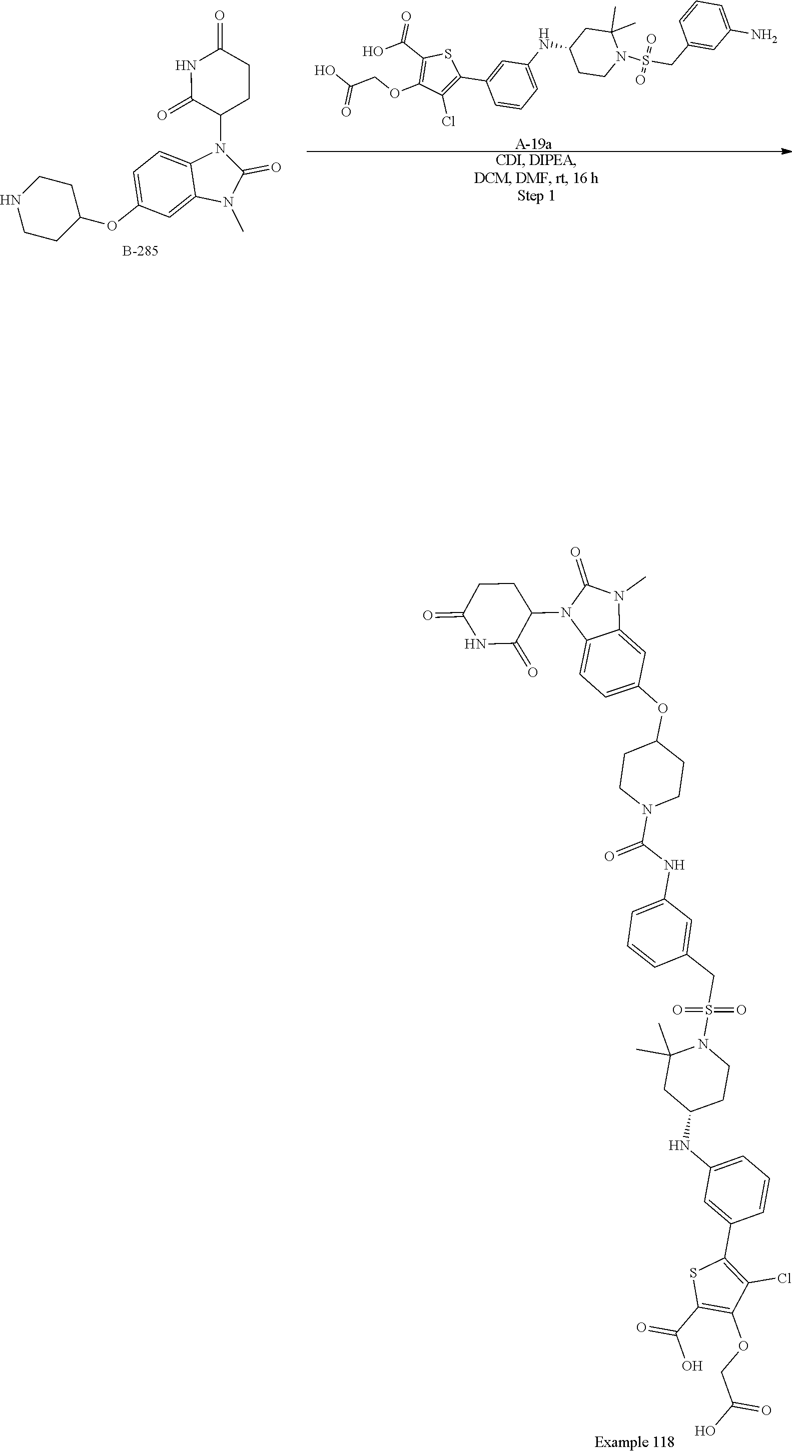 [SMILES: Cn1c(=O)n(C2CCC(=O)NC2=O)c2ccc(OC3CCNCC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(OC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[4-[1-(2,6-dioxo-3-piperidyl)-3-methyl-2-oxo-benzimidazol-5-yl]oxypiperidine-1-carbonyl]amino]phenyl]methylsulfonyl]-2,2-dimethyl-4-piperidyl]amino]phenyl]thiophene-2-carboxylic acid (118)

3-(carboxymethoxy)-4-chloro-5-[3-[[(4S)-1-[[3-[4-[1-(2,6-dioxo-3-piperidyl)-3-methyl-2-oxo-benzimidazol-5-yl]oxypiperidine-1-carbonyl]amino]phenyl]methylsulfonyl]-2,2-dimethyl-4-piperidyl]amino]phenyl]thiophene-2-carboxylic acid (118, 14 mg, 12.25 μmol, 12% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-methyl-2-oxo-5-(4-piperidyloxy)benzimidazol-1-yl]piperidine-2,6-dione (B-285) in a similar fashion to Example 23, except for using excess DIPEA as a base and DCM:DMF as the solvent. The residue was purified by reverse phase prep HPLC [Purification method: Column: XBridge, C18 (150×19 mm), 5 μm; Mobile phase A: 0.1% TFA in water and Mobile phase B: MeCN]. MS (ES+): m/z 992.2 [M+H]⁺. ¹H NMR (400 MHz, DMSO-d6): δ 11.09 (s, 1H), 8.65 (s, 1H), 7.55 (s, 1H), 7.45 (d, J=8.80 Hz, 1H), 7.25-7.09 (m, 4H), 7.01-6.92 (m, 3H), 6.87-6.80 (m, 2H), 6.70-6.68 (m, 2H), 5.70 (s, 1H), 5.35-5.30 (m, 1H), 4.92 (s, 2H), 4.56 (s, 1H), 4.31 (dd, J=13.60, 43.20 Hz, 2H), 3.81-3.59 (m, 2H), 3.32 (s, 3H), 3.22-3.09 (m, 2H), 2.94-2.90 (m, 1H), 2.08-1.77 (m, 6H), 1.62-1.59 (m, 2H), 1.49-1.40 (m, 8H), 1.15-1.00 (m, 1H).

### Example 119

### 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (119)

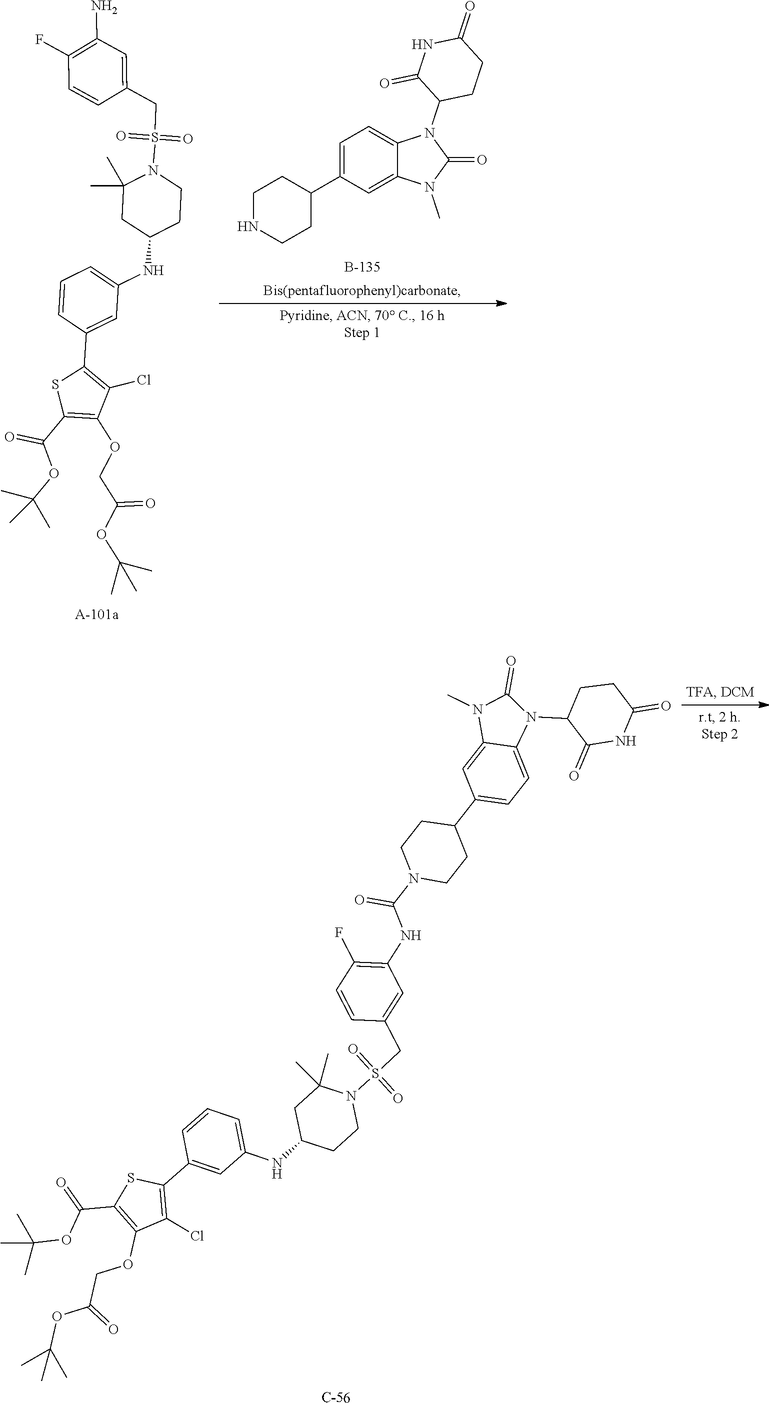

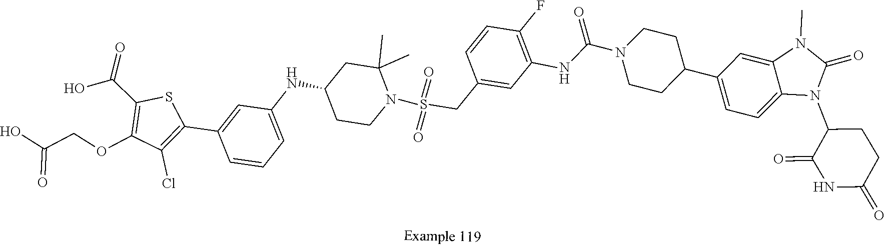 [SMILES: Cn1c(=O)n(C2CCC(=O)NC2=O)c2ccc(C3CCN(C(=O)Nc4cc(CS(=O)(=O)N5CC[C@H](Nc6cccc(-c7sc(C(=O)O)c(OCC(=O)O)c7Cl)c6)CC5(C)C)ccc4F)CC3)cc21 ; "Example 119"]

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylate (C-56)

Into a 5 mL single neck round bottom flask containing a well-stirred solution of tert-butyl 5-[3-[[(4S)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101a, 70 mg, 94.81 μmol) in anhydrous acetonitrile (3 mL) were added bis(pentafluorophenyl)carbonate (74.73 mg, 189.62 μmol) and pyridine (22.50 mg, 284.43 μmol, 23.00 μL). After 1 h, a solution of 3-[3-methyl-2-oxo-5-(4-piperidyl)benzimidazol-1-yl]piperidine-2,6-dione (B-135, 32.46 mg, 94.81 μmol) in anhydrous acetonitrile (3 mL) and pyridine (22.50 mg, 284.43 μmol, 23.00 μL) was added. The reaction mixture was heated at 70° C. for 16 h. The volatiles were removed under reduced pressure, and the residue was purified by reverse phase prep HPLC [Purification method: Column: X-Bridge C18 (19×150 mm) 5.0 μm, Mobile phase A: 0.1% TFA in water and mobile phase B: Acetonitrile] to afford tert-butyl 3-(2-tert-butoxy-2-oxo-ethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylate (C-56, 10 mg, 6.32 μmol, 7% yield, TFA salt) as an off-white solid. LCMS (ES+): m/z 1106.6 [M+H]⁺.

### Step 2: 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (119)

3-(carboxymethoxy)-4-chloro-5-[3-[[(4S)-1-[3-[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (119, 5.5 mg, 4.48 μmol, 50% yield, TFA salt) was synthesized from tert-butyl 3-(2-tert-butoxy-2-oxo-ethoxy)-4-chloro-5-[3-[[(4S)-1-[3-[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylate (C-56) in a similar fashion to Compound C-1, except using 290 eq. of TFA. The residue was triturated with diethyl ether. LCMS (ES+): m/z 994.2 [M+H]⁺. ¹H NMR (400 MHz, DMSO-d₆) δ 11.08 (s, 1H), 8.37 (s, 1H), 7.54-7.48 (m, 1H), 7.29-7.13 (m, 5H), 7.13-7.11 (m, 1H), 7.02 (d, J=8.0 Hz, 1H), 6.93 (d, J=8.3 Hz, 1H), 6.82 (s, 1H), 6.79-6.72 (m, 1H), 6.67-6.63 (m, 1H), 5.61 (d, J=8.1 Hz, 1H), 5.34 (dd, J=12.7, 5.5 Hz, 1H), 4.63 (s, 2H), 4.42 (d, J=13.9 Hz, 1H), 4.35-4.23 (m, 3H), 3.16-3.06 (m, 2H), 2.97-2.84 (m, 4H), 2.83-2.70 (m, 2H), 2.06-1.95 (m, 1H), 1.89-1.75 (m, 5H), 1.71-1.58 (m, 3H), 1.50 (s, 3H), 1.40-1.34 (m, 5H), 1.23 (s, 3H), 1.09-0.95 (m, 1H).

### Example 120

### 3-(carboxymethoxy)-4-chloro-5-[3-[(4R)-1-[3-[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (120)

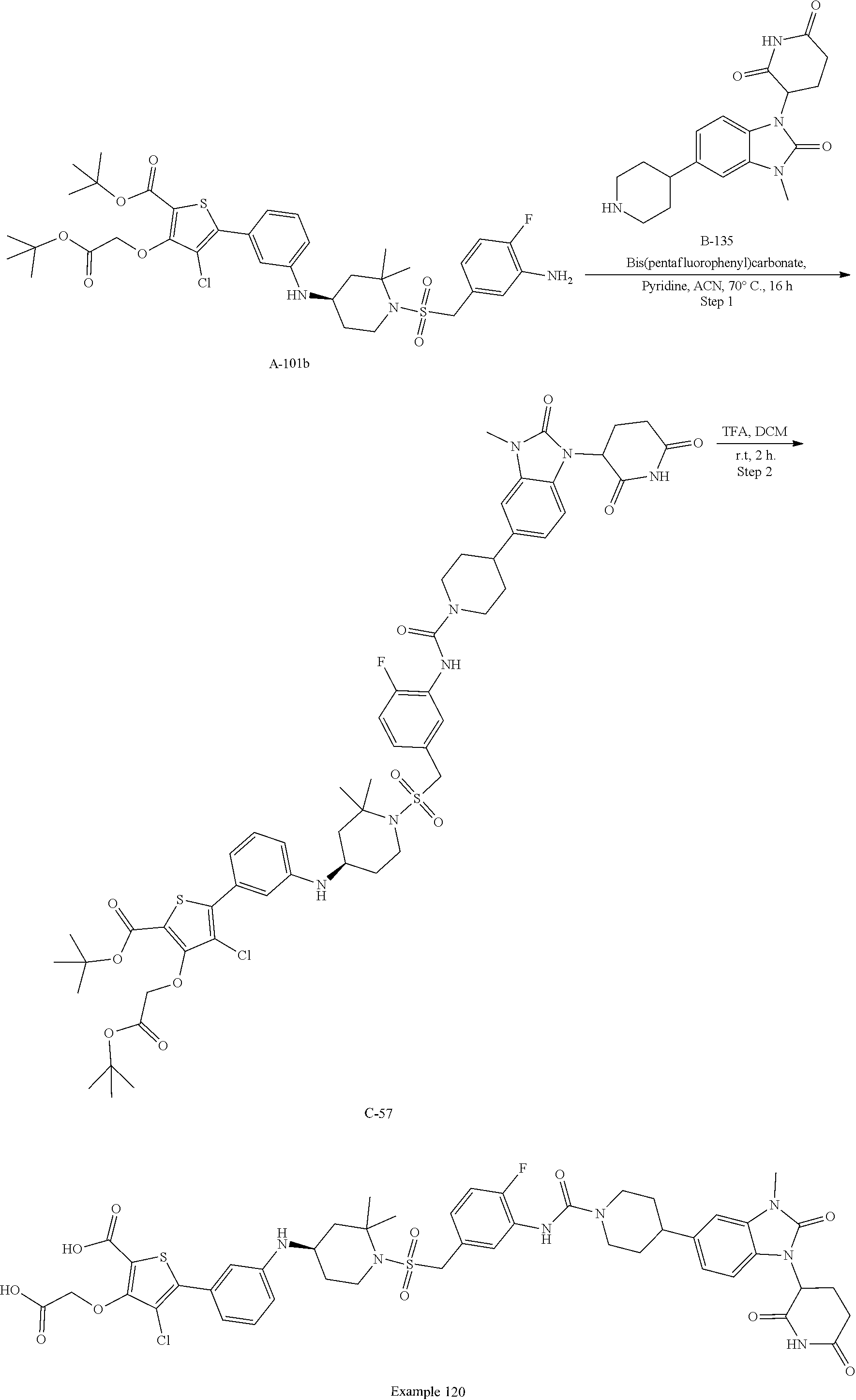

3-(carboxymethoxy)-4-chloro-5-[3-[[(4R)-1-[[3-[[4-[1-(2,6-dioxo-3-piperidyl)-3-methyl-2-oxo-benzimidazol-5-yl]piperidine-1-carbonyl]amino]-4-fluoro-phenyl]methylsulfonyl]-2,2-dimethyl-4-piperidyl]amino]phenyl]thiophene-2-carboxylic acid (120, 24 mg, 20.78 μmol, 77% yield, TFA salt) was synthesized from tert-butyl 5-[3-[(4R)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101b) and 3-[3-methyl-2-oxo-5-(4-piperidyl)benzimidazol-1-yl]piperidine-2,6-dione (B-135) over two steps following the same procedure used for the synthesis of Example 119. LCMS (ES+): m/z 994.2 [M+H]⁺. ¹H NMR (400 MHz, DMSO-d₆) δ 11.09 (s, 1H), 8.38 (s, 1H), 7.54-7.49 (m, 1H), 7.24-7.13 (m, 3H), 7.11 (s, 1H), 7.04-6.98 (m, 1H), 6.97-6.91 (m, 1H), 6.88-6.85 (m, 1H), 6.80 (d, J=7.6 Hz, 1H), 6.71-6.66 (m, 1H), 5.66 (s, 1H), 5.34 (dd, J=12.7, 5.4 Hz, 1H), 4.89 (s, 2H), 4.43 (d, J=13.9 Hz, 1H), 4.35-4.23 (m, 3H), 3.11 (t, J=12.6 Hz, 2H), 2.97-2.84 (m, 3H), 2.82-2.69 (m, 2H), 2.65-2.57 (m, 1H), 2.04-1.95 (m, 1H), 1.87-1.74 (m, 5H), 1.71-1.57 (m, 3H), 1.50 (s, 3H), 1.39 (s, 3H), 1.37-1.30 (m, 2H), 1.07-0.95 (m, 1H).

### Example 121

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-4-fluoro-phenyl]thiophene-2-carboxylic acid (121)

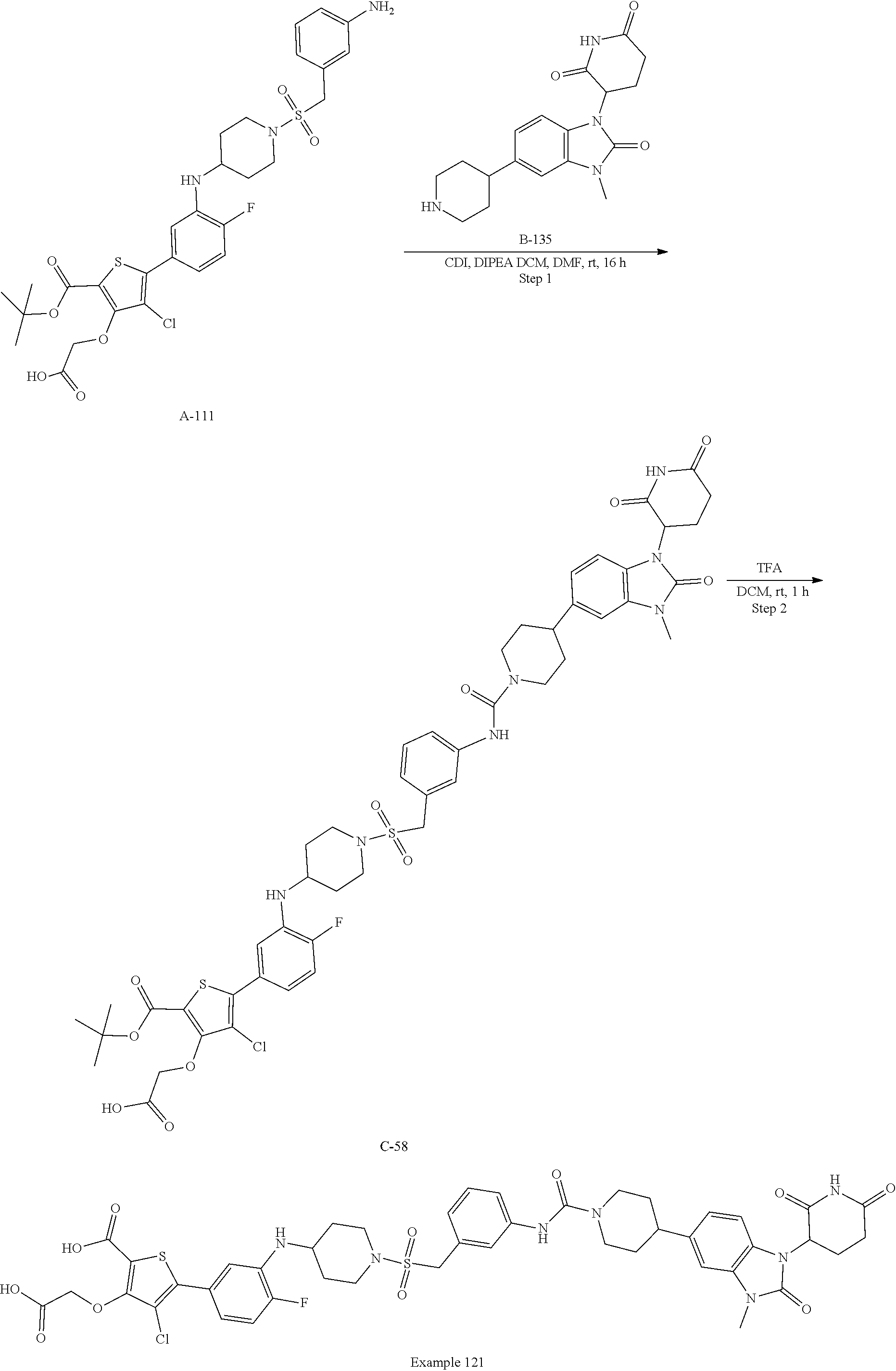

### Step 1: 2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-4-fluoro-phenyl]-3-thienyl]oxy]acetic acid (C-58)

2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-4-fluoro-phenyl]-3-thienyl]oxy]acetic acid (C-58, 110 mg, 61.08 μmol, 40% yield, 57% purity) was synthesized from 2-[[5-[3-[[1-[(3-aminophenyl)methylsulfonyl]-4-piperidyl]amino]-4-fluoro-phenyl]-2-tert-butoxycarbonyl-4-chloro-3-thienyl]oxy]acetic acid and (A-111) 3-[3-methyl-2-oxo-5-(4-piperidyl)benzimidazol-1-yl]piperidine-2,6-dione (B-135) in a similar fashion to Example 23, except excess DIPEA was used as base and DCM:DMF was the solvent. LCMS (ES−): m/z 1020.2 [M−H]⁻.

### Step 2: 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-4-fluoro-phenyl]thiophene-2-carboxylic acid (121)

3-(carboxymethoxy)-4-chloro-5-[3-[[1-[[3-[[4-[1-(2,6-dioxo-3-piperidyl)-3-methyl-2-oxo-benzimidazol-5˜yl]piperidine-1-carbonyl]amino]phenyl]methylsulfonyl]-4-piperidyl]amino]-4-fluoro-phenyl]thiophene-2-carboxylic acid (121, 17.5 mg, 15.81 μmol, 26% yield, TFA salt) was synthesized from 2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-4-fluoro-phenyl]-3-thienyl]oxy]acetic acid (C-58) in a similar fashion to Compound C-1, except using 2 eq. of TFA. The residue was purified by reverse phase column chromatography [Purification method: Biotage 30 g (C18); Mobile phase A: 0.1% TFA in MQ-water; Mobile phase B: Acetonitrile; Flow rate 10.0 mL/min]. LCMS (ES+): m/z 966.1 [M+H]⁺. ¹H NMR (400 MHz, DMSO-d6): δ 11.09 (s, 1H), 8.62 (s, 1H), 7.61 (s, 1H), 7.43 (d, J=8.40 Hz, 1H), 7.25-7.09 (m, 4H), 7.03-6.92 (m, 4H), 6.82 (s, 1H), 5.59-5.57 (m, 1H), 5.37-5.32 (m, 1H), 4.85 (s, 2H), 4.34-4.28 (m, 4H), 3.59-3.50 (m, 2H), 2.99-2.65 (m, 6H), 2.00-1.80 (m, 4H), 1.76-1.24 (m, 8H).

### Example 122

### 3-(carboxymethoxy)-4-chloro-5-[3-[[(4S)-1-[[3-[1-[1-(2,6-dioxo-3-piperidyl)-3-methyl-2-oxo-benzimidazol-S-yl]piperidine-4-carbonyl]amino]phenyl]methylsulfonyl]-2,2-dimethyl-4-piperidyl]amino]phenyl]thiophene-2-carboxylic acid (122)

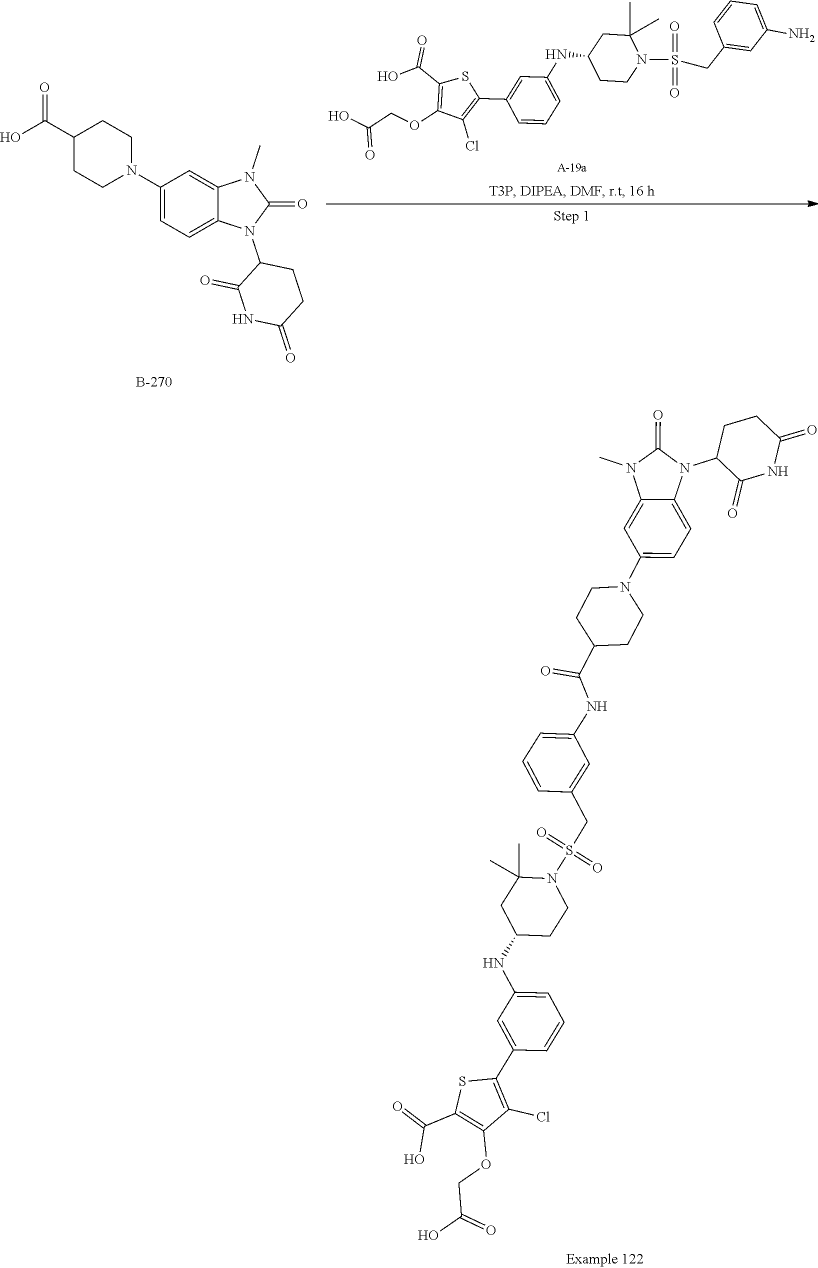 [SMILES: Cn1c(=O)n(C2CCC(=O)NC2=O)c2ccc(N3CCC(C(=O)O)CC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(N3CCC(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[1-[1-(2,6-dioxo-3-piperidyl)-3-methyl-2-oxo-benzimidazol-5-yl]piperidine-4-carbonyl]amino]phenyl]methylsulfonyl]-2,2-dimethyl-4-piperidyl]amino]phenyl]thiophene-2-carboxylic acid (122)

3-(carboxymethoxy)-4-chloro-5-[3-[[(4S)-1-[[3-[[1-[1-(2,6-dioxo-3-piperidyl)-3-methyl-2-oxo-benzimidazol-5-yl]piperidine-4-carbonyl]amino]phenyl]methylsulfonyl]-2,2-dimethyl-4-piperidyl]amino]phenyl]thiophene-2-carboxylic acid (122, 20 mg, 18.02 μmol, 7% yield, TFA salt) was synthesized from 1-[1-(2,6-dioxo-3-piperidyl)-3-methyl-2-oxo-benzimidazol-5-yl]piperidine-4-carboxylic acid (B-270) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Compound C-6, except using DIPEA as base. The residue was purified by reverse phase prep HPLC [Purification method: Column: X bridge (150×19 mm), 5 μm; Mobile phase A: 0.1% TFA in MQ-water; Mobile phase B: Acetonitrile]. LCMS (ES+): m/z 976.2 [M+H]⁺. ¹H NMR (400 MHz, DMSO-d₆) δ 11.11 (s, 1H), 10.13 (s, 1H), 7.73-7.69 (m, 1H), 7.65-7.60 (m, 1H), 7.36-7.25 (m, 2H), 7.20 (t, J=8.0 Hz, 1H), 7.16-6.99 (m, 3H), 6.89-6.85 (m, 1H), 6.83-6.79 (m, 1H), 6.72-6.67 (m, 1H), 5.36 (dd, J=12.9, 5.3 Hz, 1H), 4.90 (s, 2H), 4.41 (d, J=13.7 Hz, 1H), 4.30 (d, J=13.7 Hz, 1H), 3.73-3.65 (m, 2H), 3.36 (s, 3H), 3.18-3.08 (m, 2H), 2.95-2.83 (m, 1H), 2.77-2.59 (m, 3H), 2.10-1.95 (m, 5H), 1.93-1.85 (m, 1H), 1.80 (d, J=12.8 Hz, 1H), 1.48 (s, 3H), 1.45-1.37 (m, 4H), 1.28-1.22 (m, 1H), 1.18-1.05 (m, 1H), 0.99-0.82 (m, 1H).

### Example 123

### 3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-2-fluoro-phenyl]thiophene-2-carboxylic acid (123)

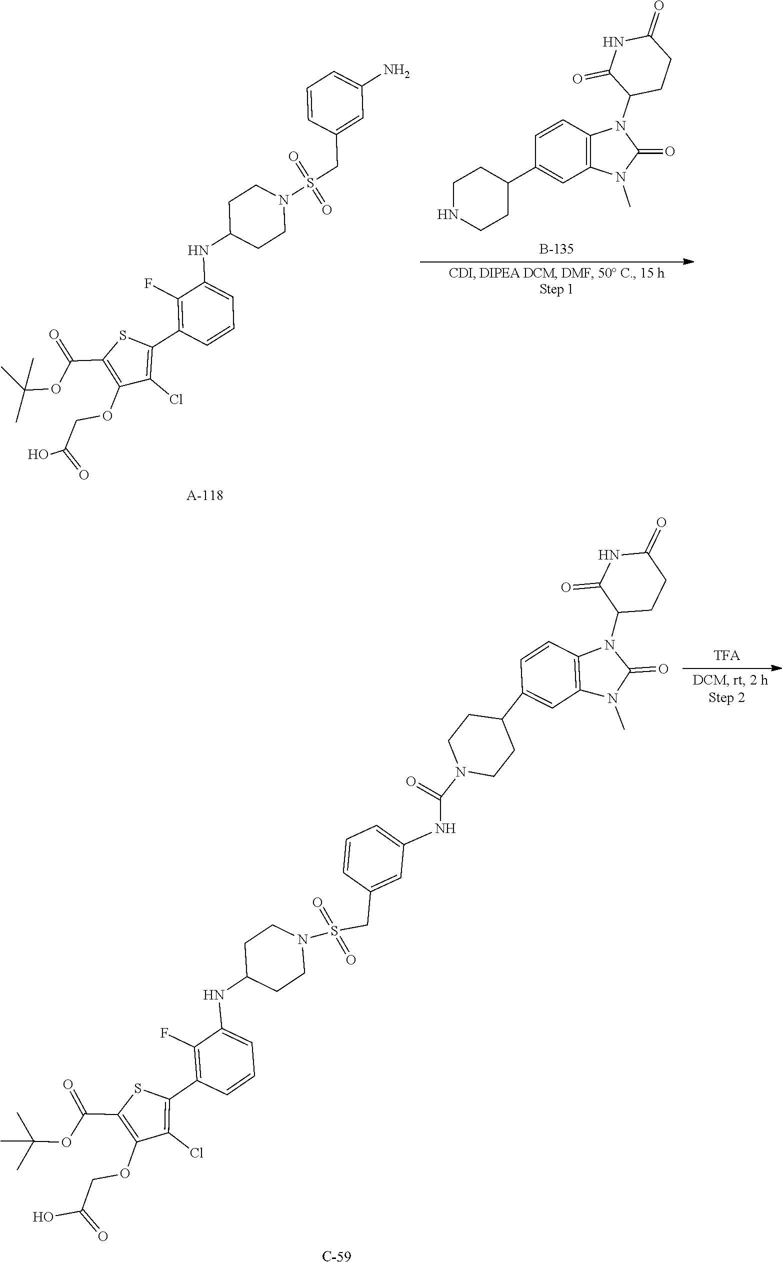

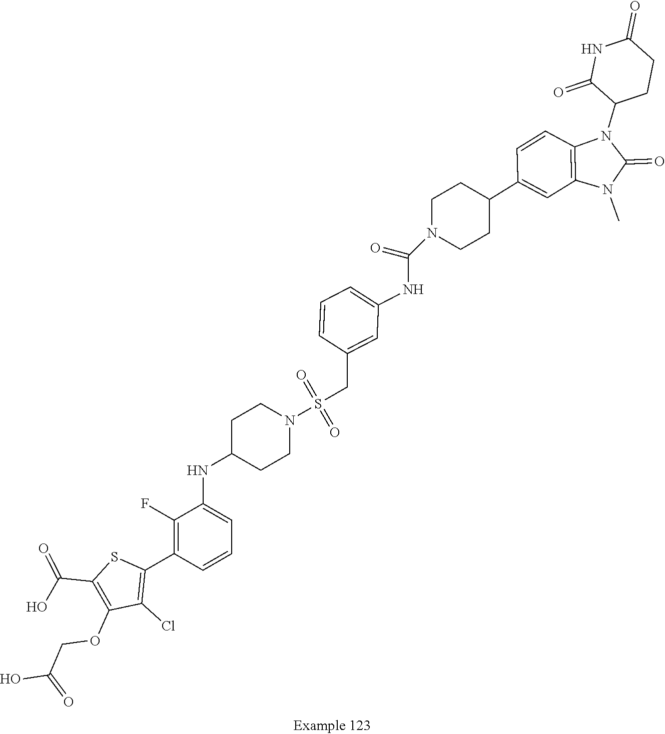 [SMILES: Cn1c(=O)n(C2CCC(=O)NC2=O)c2ccc(C3CCN(C(=O)Nc4cccc(CS(=O)(=O)N5CCC(Nc6cccc(-c7sc(C(=O)O)c(OCC(=O)O)c7Cl)c6F)CC5)c4)CC3)cc21]

### Step 1: 2-[[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-2-fluoro-phenyl]-3-thienyl]oxy]acetic acid (C-59)

2-[[2-tert-butoxycarbonyl-4-chloro-5-[3-[[1-[[3-[[4-[1-(2,6-dioxo-3-piperidyl)-3-methyl-2-oxo-benzimidazol-S-yl]piperidine-1-carbonyl]amino]phenyl]methylsulfonyl]-4-piperidyl]amino]-2-fluoro-phenyl]-3-thienyl]oxy]acetic acid (C-59, 100 mg, 70.41 μmol, 70% yield) was synthesized from 2-[[5-[3-[[1-[(3-aminophenyl)methylsulfonyl]-4-piperidyl]amino]-2-fluoro-phenyl]-2-tert-butoxycarbonyl-4-chloro-3-thienyl]oxy]acetic acid (A-118) and 3-[3-methyl-2-oxo-5-(4-piperidyl)benzimidazol-1-yl]piperidine-2,6-dione (B-135) in a similar fashion to Example 23, except using excess DIPEA as base, DCM:DMF as solvent and heating the reaction to 50° C. LCMS (ES+): m/z 966.2 [M-tBu+H]⁺.

### Step 2: 3-(carboxymethoxy)-4-chloro-5-[3-[1-[[3-[[4-[1-(2,6-dioxo-3-piperidyl)-3-methyl-2-oxo-benzimidazol-S-yl]piperidine-1-carbonyl]amino]phenyl]methylsulfonyl]-4-piperidyl]amino]-2-fluoro-phenyl]thiophene-2-carboxylic acid (123)

3-(carboxymethoxy)-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-2-fluoro-phenyl]thiophene-2-carboxylic acid (123, 22 mg, 20.97 μmol, 30% yield, formic acid salt) was synthesized from 2-[2-tert-butoxycarbonyl-4-chloro-5-[3-[[1-[[3-[[4-[1-(2,6-dioxo-3-piperidyl)-3-methyl-2-oxo-benzimidazol-5-yl]piperidine-1-carbonyl]amino]phenyl]methylsulfonyl]-4-piperidyl]amino]-2-fluoro-phenyl]-3-thienyl]oxy]acetic acid (C-59) in a similar fashion to Compound C-1 except using 5 eq. of TFA. The residue was purified by reverse phase column chromatography [Purification Method: Biotage 30 g (C18), mobile phase A: 0.1% formic acid in MQ-water and mobile phase B: MeCN, (FR-15.0 mL/min)]. LCMS (ES+): m/z 966.5 [M+H]⁺. ¹H NMR (400 MHz, DMSO-d₆): δ 11.09 (s, 1H), 8.64 (s, 1H), 7.61 (s, 1H), 7.44 (d, J=8.80 Hz, 1H), 7.28-7.21 (m, 2H), 7.14 (s, 1H), 7.10-6.85 (m, 6H), 6.63 (t, J=6.40 Hz, 1H), 5.54 (d, J=7.60 Hz, 1H), 5.34 (dd, J=5.20, 12.80 Hz, 1H), 4.72 (s, 2H), 4.36-4.32 (m, 4H), 3.62-3.55 (m, 3H), 2.94-2.86 (m, 6H), 2.60-2.80 (m, 3H), 2.02-1.99 (m, 1H), 1.92-1.89 (m, 2H), 1.82-1.77 (m, 2H), 1.66-1.64 (m, 2H), 1.49-1.47 (m, 2H).

### Example 124

### 3-(carboxymethoxy)-4-chloro-5-[3-[[(4S)-1-[[3-[[4-[3-(2,6-dioxo-3-piperidyl)-2-oxo-benzimidazol-1-yl]piperidine-1-carbonyl]amino]phenyl]methylsulfonyl]-2,2-dimethyl-4-piperidyl]amino]phenyl]thiophene-2-carboxylic acid (124)

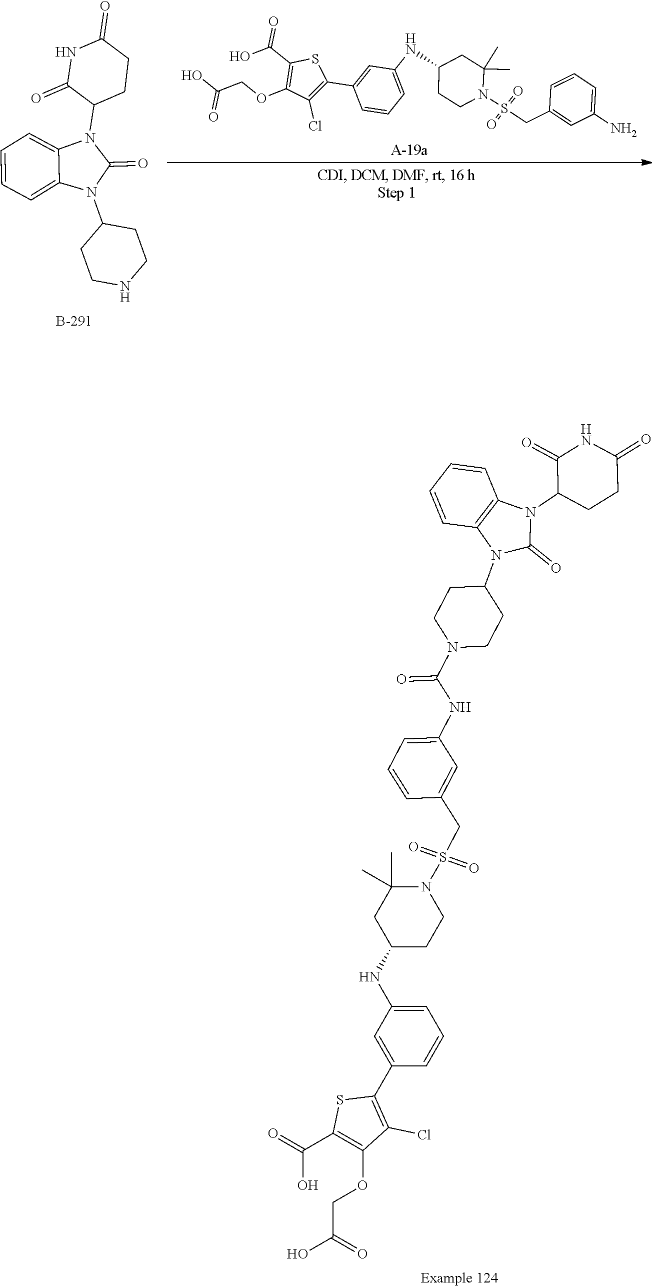 [SMILES: O=C1CCC(n2c(=O)n(C3CCNCC3)c3ccccc32)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(n3c(=O)n(C4CCC(=O)NC4=O)c4ccccc43)CC2)c1]

### Step 1: 3-(carboxymethoxy)-4-chloro-5-[3-[[(4S)-1-[3-[4-[3-(2,6-dioxo-3-piperidyl)-2-oxo-benzimidazol-1-yl]piperidine-1-carbonyl]amino]phenyl]methylsulfonyl]-2,2-dimethyl-4-piperidyl]amino]phenyl]thiophene-2-carboxylic acid (124)

3-(carboxymethoxy)-4-chloro-5-[3-[[(4S)-1-[3-[4-[3-(2,6-dioxo-3-piperidyl)-2-oxo-benzimidazol-1-yl]piperidine-1-carbonyl]amino]phenyl]methylsulfonyl]-2,2-dimethyl-4-piperidyl]amino]phenyl]thiophene-2-carboxylic acid (124, 21 mg, 19.31 μmol, 15% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[2-oxo-3-(4-piperidyl)benzimidazol-1-yl]piperidine-2,6-dione (B-291) in a similar fashion to Example 23, except using DCM:DMF as the solvent. The residue was purified by reverse phase prep HPLC [Purification method: Column: Agilent, C18 (50×21.2 mm), 5 μm; Mobile phase A: 0.1% TFA in water and Mobile phase B: MeCN]. LCMS (ES+): m/z 962.6 [M+H]⁺. ¹H NMR (400 MHz, DMSO-d6): δ 11.11 (s, 1H), 8.71 (s, 1H), 7.57 (s, 1H), 7.48 (d, J=8.40 Hz, 1H), 7.33-7.18 (m, 3H), 7.13-6.98 (m, 4H), 6.87 (s, 1H), 6.81 (d, J=7.60 Hz, 1H), 6.70-6.68 (m, 1H), 5.39-5.34 (m, 1H), 4.91 (s, 2H), 4.46-4.25 (m, 6H), 3.10-2.86 (m, 5H), 2.72-2.60 (m, 3H), 2.34-2.29 (m, 3H), 2.09-2.01 (m, 1H), 1.88-1.76 (m, 4H), 1.49-1.40 (m, 7H), 1.15-1.00 (m, H).

### Example 125

### 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (125)

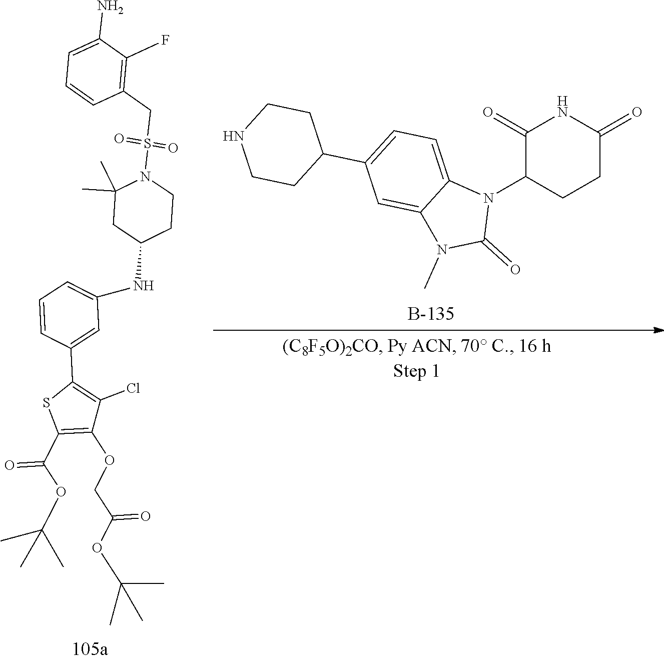 [SMILES: CC(C)(C)OC(=O)COc1c(C(=O)OC(C)(C)C)sc(-c2cccc(N[C@H]3CCN(S(=O)(=O)Cc4cccc(N)c4F)C(C)(C)C3)c2)c1Cl ; Cn1c(=O)n(C2CCC(=O)NC2=O)c2ccc(C3CCNCC3)cc21]

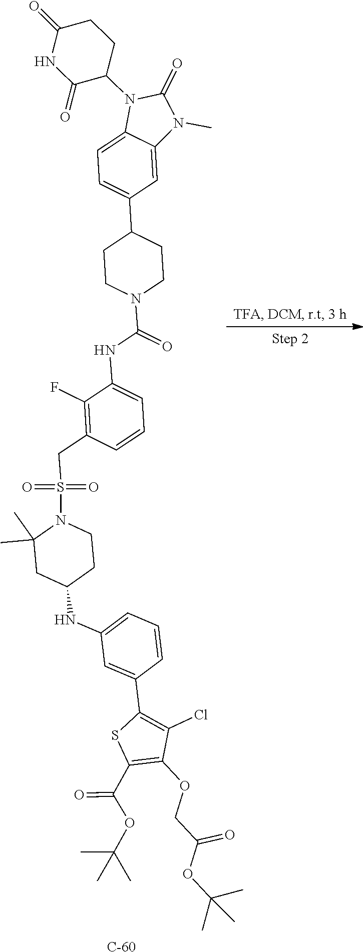

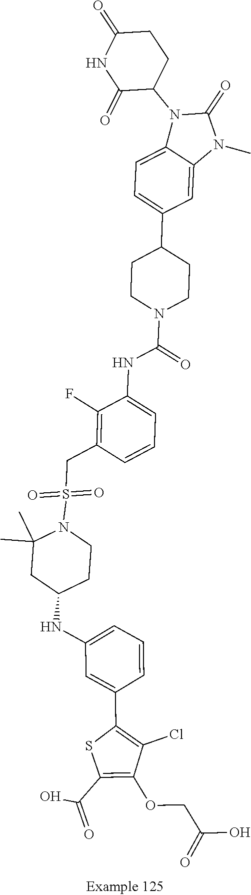

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylate (C-60)

Into a 10 mL round bottom single neck flask containing a well-stirred solution of tert-butyl 5-[3-[[(4S)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105a, 80 mg, 108.35 μmol) in anhydrous acetonitrile (0.3 mL) were added bis(pentafluorophenyl)carbonate (85.41 mg, 216.71 μmol) and pyridine (25.71 mg, 325.06 μmol, 26.29 μL). The reaction mixture was stirred at 50° C. for 1 h. Then a solution of 3-[3-methyl-2-oxo-5-(4-piperidyl)benzimidazol-1-yl]piperidine-2,6-dione (B-135, 59.34 mg, 130.02 μmol) in anhydrous acetonitrile (0.3 mL) and pyridine (25.71 mg, 325.06 μmol, 26.29 μL) was added to the reaction mixture at room temperature. The resulting mixture was stirred at 75° C. for 16 h. The volatiles were removed under reduced pressure and the residue was purified by reverse phase column chromatography [Purification method: Column: X bridge (150×19 mm), 5 μm; Mobile phase A: 0.1% TFA in MQ-water; Mobile phase B: Acetonitrile] to afford tert-butyl 3-(2-tert-butoxy-2-oxo-ethoxy)-4-chloro-5-[3-[[(4S)-1-[[3-[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylate (C-60, 15 mg, 12.22 μmol, 11% yield, TFA salt) as an off-white solid. LCMS (ESI): m/z 1106.6 [M+H]⁺.

### Step 2: 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (125)

3-(carboxymethoxy)-4-chloro-5-[3-[[(4S)-1-[3-[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (125, 10.98 mg, 9.88 μmol, 68% yield, TFA salt) was synthesized from of tert-butyl 3-(2-tert-butoxy-2-oxo-ethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylate (C-60) in a similar fashion to Compound C-1, except using 460 eq. of TFA. The residue was triturated with diethyl ether. LCMS (ESI): m/z 994.3 [M+H]⁺. ¹H NMR (400 MHz, DMSO-d₆) δ 11.08 (s, 1H), 8.39 (s, 1H), 7.46-7.39 (m, 1H), 7.25-7.09 (m, 4H), 7.04-6.97 (m, 1H), 6.93 (d, J=8.1 Hz, 1H), 6.88-6.84 (m, 1H), 6.80 (d, J=7.5 Hz, 1H), 6.71-6.65 (m, 1H), 5.66 (s, 1H), 5.34 (dd, J=12.7, 5.4 Hz, 1H), 4.87 (s, 2H), 4.47 (d, J=14.0 Hz, 1H), 4.38 (d, J=13.9 Hz, 1H), 4.28 (d, J=12.8 Hz, 2H), 3.60-3.49 (m, 2H), 3.19-3.08 (m, 2H), 2.99-2.85 (m, 3H), 2.82-2.57 (m, 4H), 2.05-1.96 (m, 1H), 1.86-1.73 (m, 4H), 1.71-1.59 (m, 2H), 1.52 (s, 3H), 1.49-1.36 (m, 5H), 1.25 (dd, J=11.0, 4.6 Hz, 2H), 1.10-0.97 (m, 1H).

### Example 126

### 3-(carboxymethoxy)-4-chloro-5-[3-[[(4S)-1-[3-[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylic acid (126)

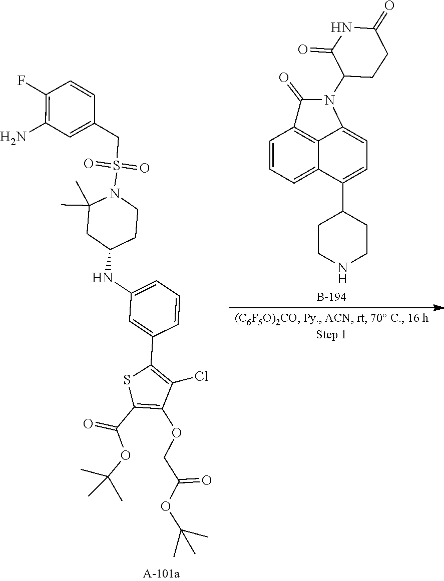 [SMILES: CC(C)(C)OC(=O)COc1c(C(=O)OC(C)(C)C)sc(-c2cccc(N[C@H]3CCN(S(=O)(=O)Cc4ccc(F)c(N)c4)C(C)(C)C3)c2)c1Cl ; O=C1CCC(N2C(=O)c3cccc4c(C5CCNCC5)ccc2c34)C(=O)N1]

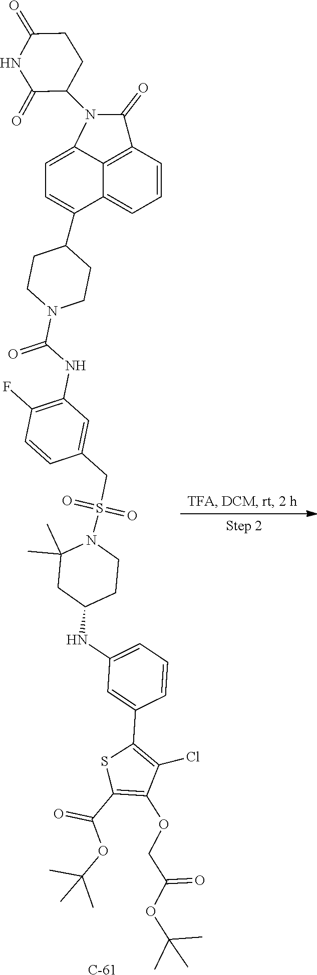

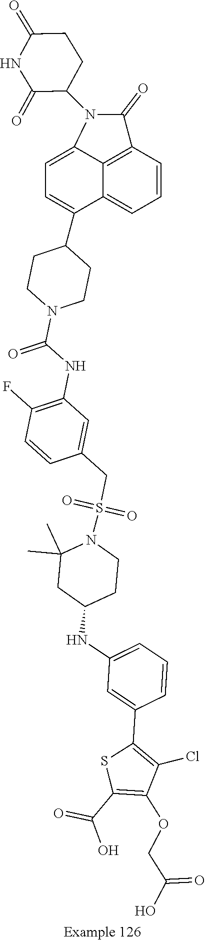 [SMILES: CC1(C)C[C@@H](Nc2cccc(-c3sc(C(=O)O)c(OCC(=O)O)c3Cl)c2)CCN1S(=O)(=O)Cc1ccc(F)c(NC(=O)N2CCC(c3ccc4c5c(cccc35)C(=O)N4C3CCC(=O)NC3=O)CC2)c1 ; "Example 126"]

### Step 1: tert-butyl 3-(2-tert-butoxy-2-oxo-ethoxy)-4-chloro-5-[3-[[(4S)-1-∥3-[[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylate (C-61)

Tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylate (C-61, 250 mg, 41.43 μmol, 20% yield) was synthesized from tert-butyl 5-[3-[[(4S)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101a) and 3-[2-oxo-6-(4-piperidyl)benzo[cd]indol-1-yl]piperidine-2,6-dione (B-194) in a similar fashion to Example 125. The material was taken to next step without any purification. LCMS (ES+): m/z 1126.6 [M+H]⁺.

### Step 2: 3-(carboxymethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylic acid (126)

3-(carboxymethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylic acid (126, 10 mg, 8.61 μmol, 21% yield, TFA salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylate (C-61) in a similar fashion to Compound C-1 except using 325 eq. of TFA. The residue was purified by reverse phase prep HPLC [Purification method: Column: XSelect C18 (150×19 mm) 5 μm; Mobile phase: 0.1% TFA in water, Mobile phase B: MeCN]. LCMS (ES−): m/z 1012.7 [M−H]⁻. ¹H NMR (400 MHz, DMSO-d₆) δ 11.13 (s, 1H), 8.48 (d, J=8.4 Hz, 1H), 8.43 (s, 1H), 8.11 (d, J=6.9 Hz, 1H), 7.91-7.82 (m, 1H), 7.51 (dd, J=7.7, 2.1 Hz, 1H), 7.36 (d, J=7.5 Hz, 1H), 7.27-7.06 (m, 4H), 6.87-6.84 (m, 1H), 6.79 (d, J=7.5 Hz, 1H), 6.71-6.66 (m, 1H), 5.42 (dd, J=12.8, 5.5 Hz, 1H), 4.88 (s, 2H), 4.42 (d, J=13.9 Hz, 1H), 4.35-4.25 (m, 3H), 3.15-3.04 (m, 4H), 2.99-2.87 (m, 2H), 2.81-2.61 (m, 3H), 2.13-2.03 (m, 1H), 1.96-1.64 (m, 6H), 1.49 (s, 3H), 1.44-1.34 (m, 4H), 1.09-0.95 (m, 1H).

### Example 127

### 3-(carboxymethoxy)-4-chloro-5-[3-[(4R)-1-[3-[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylic acid (127)

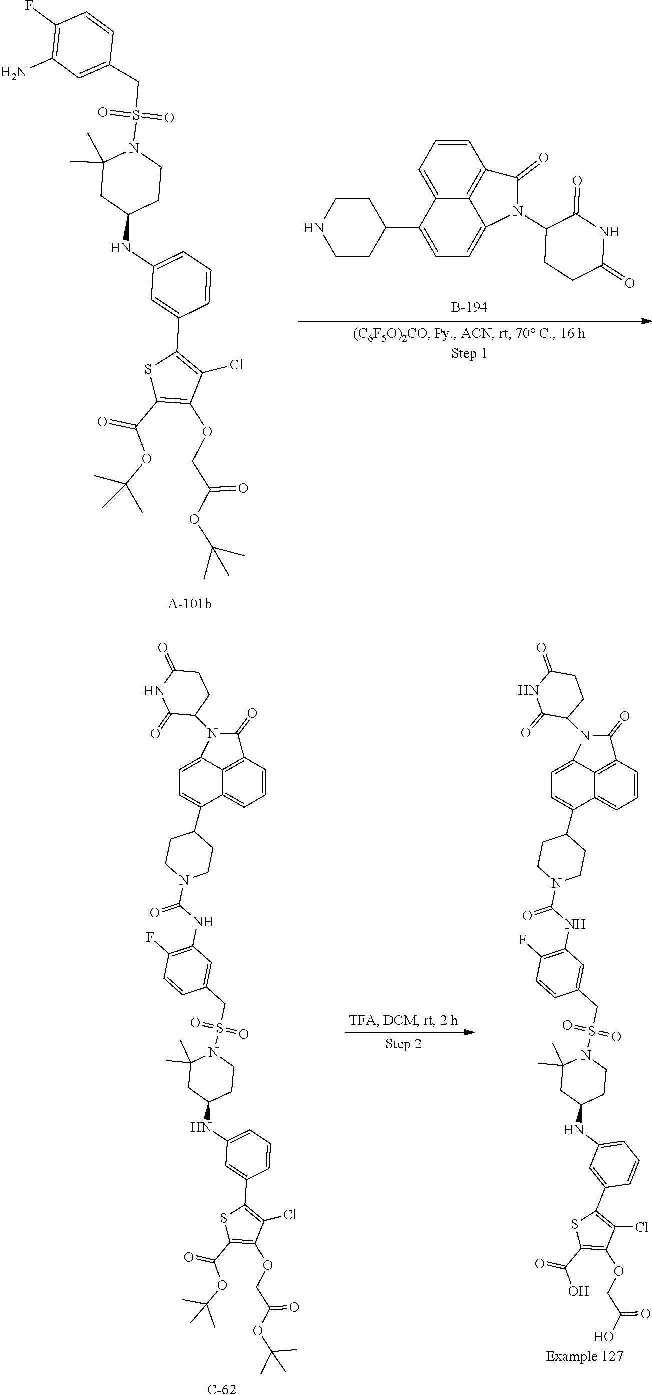

3-(carboxymethoxy)-4-chloro-5-[3-[(4R)-1-[[3-[[4-[1-(2,6-dioxo-3-piperidyl)-2-oxo-benzo[cd]indol-6-yl]piperidine-1-carbonyl]amino]-4-fluoro-phenyl]methylsulfonyl]-2,2-dimethyl-4-piperidyl]amino]phenyl]thiophene-2-carboxylic acid (127, 10 mg, 8.49 μmol, 26% yield, TFA salt) was synthesized from tert-butyl 5-[3-[(4R)-1-[(3-amino-4-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-101b) and 3-[2-oxo-6-(4-piperidyl)benzo[cd]indol-1-yl]piperidine-2,6-dione (B-194) using the same two step procedure as Example 126. LCMS (ES−): m/z 1012.8 [M−H]⁻. ¹H NMR (400 MHz, DMSO-d₆) δ 11.12 (s, 1H), 8.48 (d, J=8.4 Hz, 1H), 8.43 (s, 1H), 8.10 (d, J=7.0 Hz, 1H), 7.91-7.84 (m, 1H), 7.50 (dd, J=7.7, 2.1 Hz, 1H), 7.35 (d, J=7.5 Hz, 1H), 7.22-7.05 (m, 4H), 6.84 (d, J=2.0 Hz, 1H), 6.82-6.76 (m, 1H), 6.70-6.65 (m, 1H), 5.41 (dd, J=12.8, 5.4 Hz, 1H), 4.86 (s, 2H), 4.41 (d, J=13.9 Hz, 1H), 4.33-4.24 (m, 3H), 3.15-3.04 (m, 4H), 2.99-2.87 (m, 2H), 2.79-2.62 (m, 3H), 2.14-2.05 (m, 1H), 1.95-1.64 (m, 6H), 1.49 (s, 3H), 1.44-1.33 (m, 4H), 1.07-0.93 (m, 1H).

### Example 128

### 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-2-fluorobenzyl)sulfonyl)-2,2-dimethylpiperidin-4-yl)amino)phenyl)thiophene-2-carboxylic acid (128)

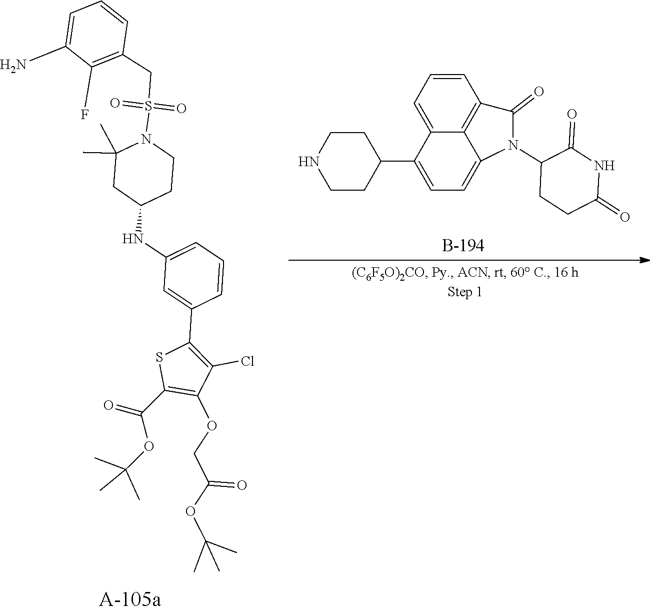 [SMILES: CC(C)(C)OC(=O)COc1c(C(=O)OC(C)(C)C)sc(-c2cccc(N[C@H]3CCN(S(=O)(=O)Cc4cccc(N)c4F)C(C)(C)C3)c2)c1Cl ; O=C1CCC(N2C(=O)c3cccc4c(C5CCNCC5)ccc2c34)C(=O)N1]

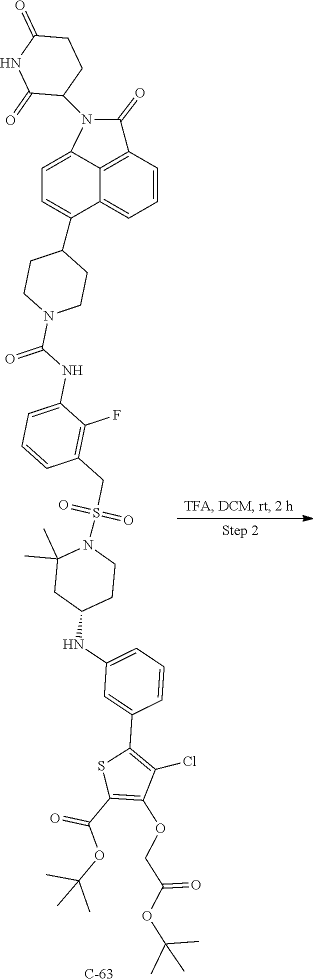

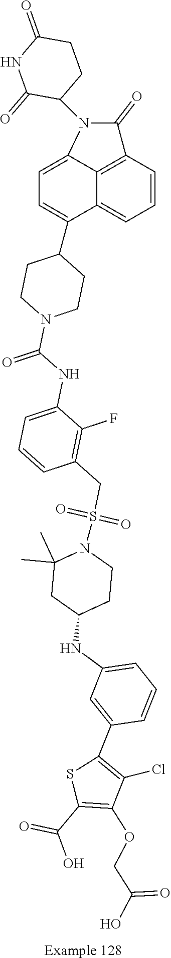 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1F ; "Example 128"]

### Step 1: tert-butyl 3-(2-(tert-butoxy)-2-oxoethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-2-fluorobenzyl)sulfonyl)-2,2-dimethylpiperidin-4-yl)amino)phenyl)thiophene-2-carboxylate (C-63)

Tert-butyl 3-(2-tert-butoxy-2-oxo-ethoxy)-4-chloro-5-[3-[[(4S)-1-[[3-[[4-[1-(2,6-dioxo-3-piperidyl)-2-Tert-butyl oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylate (C-63, 350 mg, 71.51 μmol, 26% yield) was synthesized from tert-butyl 5-[3-[[(4S)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105a) and 3-[2-oxo-6-(4-piperidyl)benzo[cd]indol-1-yl]piperidine-2,6-dione (B-194) in a similar fashion to Example 125. The crude sample was taken to next step without any purification. LCMS (ES+): m/z 1127.2 [M+Na]⁺.

### Step 2: 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-2-fluorobenzyl)sulfonyl)-2,2-dimethylpiperidin-4-yl)amino)phenyl)thiophene-2-carboxylic acid (128)

3-(carboxymethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (128, 55 mg, 48.43 μmol, 68% yield, TFA salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylate (C-63) in a similar fashion to Compound C-1 except using 185 eq. of TFA. The residue was purified by reverse phase prep HPLC [Purification method: Column: XSelect C18 (150×19 mm) 5 μm, Mobile phase A: 0.1% TFA in water and Mobile phase B: MeCN]. LCMS (ES−): m/z 1013.1 [M−H]⁻. ¹H NMR (400 MHz, DMSO-d₆) δ 11.13 (s, 1H), 8.51-8.41 (m, 2H), 8.10 (d, J=7.0 Hz, 1H), 7.87 (t, J=7.6 Hz, 1H), 7.45-7.39 (m, 1H), 7.35 (d, J=7.5 Hz, 1H), 7.24-7.05 (m, 4H), 6.87 (s, 1H), 6.81 (d, J=7.6 Hz, 1H), 6.72-6.67 (m, 1H), 5.42 (dd, J=12.7, 5.5 Hz, 1H), 4.87 (s, 2H), 4.51-4.25 (m, 5H), 3.19-3.05 (m, 4H), 2.99-2.88 (m, 1H), 2.81-2.61 (m, 3H), 2.13-2.04 (m, 1H), 1.94-1.85 (m, 2H), 1.84-1.65 (m, 4H), 1.52 (s, 3H), 1.47-1.34 (m, 4H), 1.11-0.97 (m, 1H).

### Example 129

### 3-(carboxymethoxy)-4-chloro-5-(3-(((4R)-1-((3-(4-(1-(2,6-dioxopiperidin-3-yl)-2-oxo-1,2-dihydrobenzo[cd]indol-6-yl)piperidine-1-carboxamido)-2-fluorobenzyl)sulfonyl)-2,2-dimethylpiperidin-4-yl)amino)phenyl)thiophene-2-carboxylic acid (129)

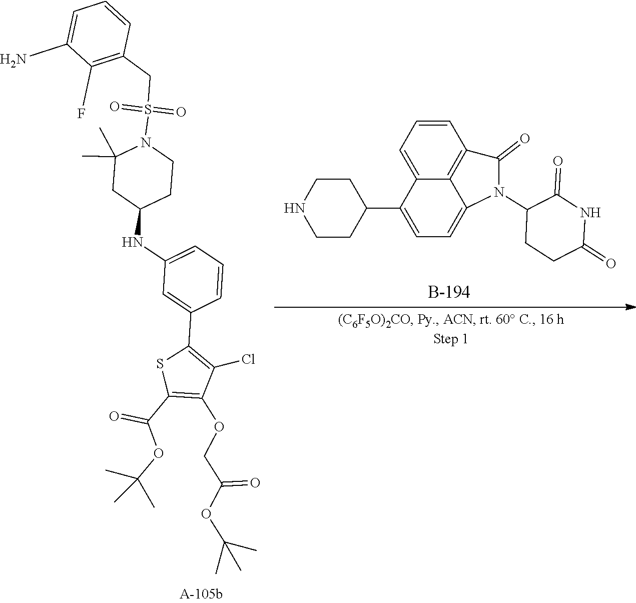 [SMILES: CC(C)(C)OC(=O)COc1c(C(=O)OC(C)(C)C)sc(-c2cccc(N[C@@H]3CCN(S(=O)(=O)Cc4cccc(N)c4F)C(C)(C)C3)c2)c1Cl ; O=C1CCC(N2C(=O)c3cccc4c(C5CCNCC5)ccc2c34)C(=O)N1]

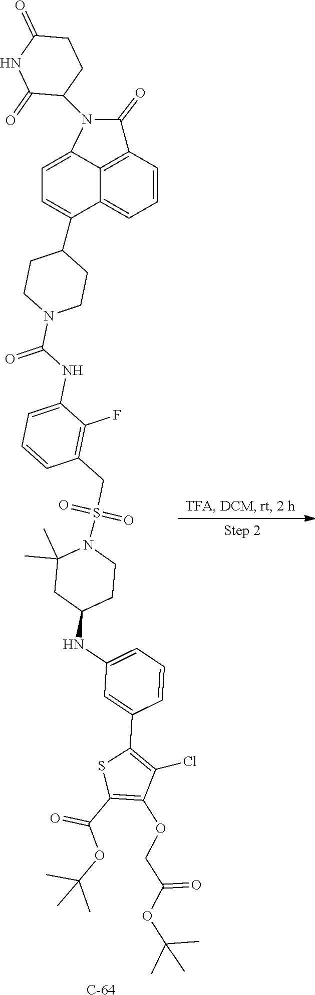

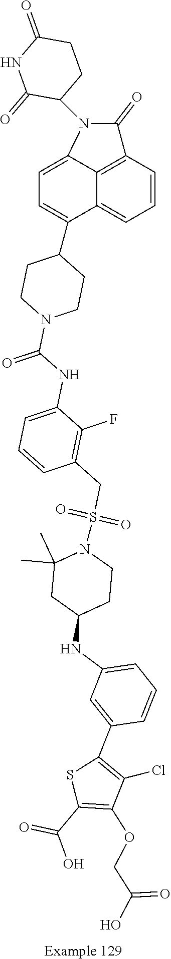 [SMILES: CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1F ; "Example 129"]

3-(carboxymethoxy)-4-chloro-5-(3-(((4R)-1-((3-(4-(1-(2,6-dioxopiperidin-3-yl)-2-oxo-1,2-dihydrobenzo[cd]indol-6-yl)piperidine-1-carboxamido)-2-fluorobenzyl)sulfonyl)-2,2-dimethylpiperidin-4-yl)amino)phenyl)thiophene-2-carboxylic acid (129, 35 mg, 30.43 μmol, 49% yield, TFA salt) was synthesized from tert-butyl 5-[3-[[(4R)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105b) and 3-[2-oxo-6-(4-piperidyl)benzo[cd]indol-1-yl]piperidine-2,6-dione (B-194) using the same two step procedure as Example 128. LCMS (ES−): m/z 1013.2 [M−H]⁻. ¹H NMR (400 MHz, DMSO-d₆) δ 11.13 (s, 1H), 8.52-8.43 (m, 2H), 8.11 (d, J=6.9 Hz, 1H), 7.87 (t, J=7.7 Hz, 1H), 7.47-7.41 (m, 1H), 7.36 (d, J=7.5 Hz, 1H), 7.26-7.06 (m, 4H), 6.87 (s, 1H), 6.80 (d, J=7.6 Hz, 1H), 6.73-6.67 (m, 1H), 5.43 (dd, J=12.9, 5.5 Hz, 1H), 4.88 (s, 2H), 4.51-4.27 (m, 5H), 3.19-3.06 (m, 4H), 3.00-2.88 (m, 1H), 2.82-2.62 (m, 3H), 2.14-2.04 (m, 1H), 1.96-1.67 (m, 6H), 1.52 (s, 3H), 1.49-1.36 (m, 4H), 1.14-0.96 (m, 1H).

### Example 130

### 3-(carboxymethoxy)-4-chloro-5-[3-[[(4S)-1-[[3-[[2-[4-[3-(2,6-dioxo-3-piperidyl)-2-oxo-benzimidazol-1-yl]-1-piperidyl]acetyl]amino]phenyl]methylsulfonyl]-2,2-dimethyl-4-piperidyl]amino]phenyl]thiophene-2-carboxylic acid (130)

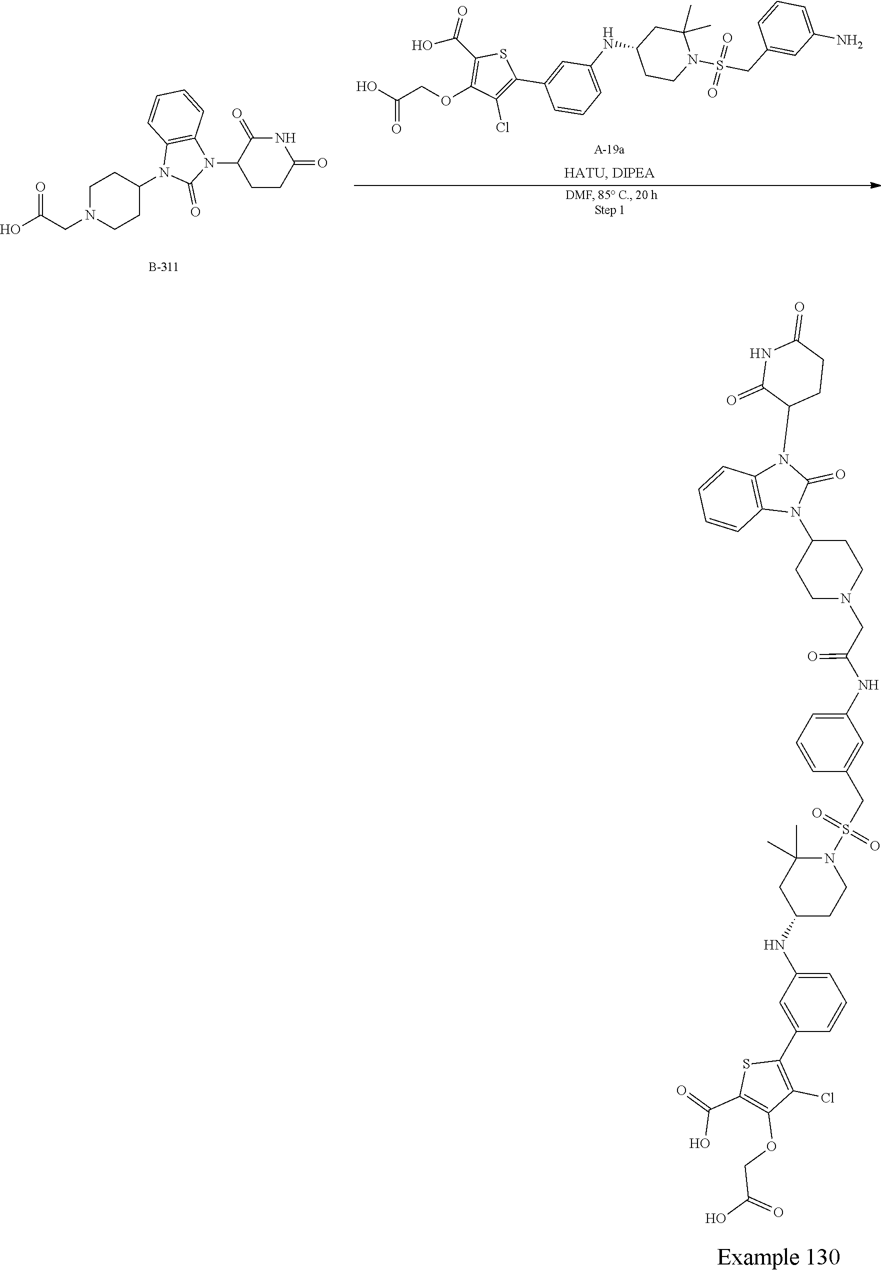 [SMILES: O=C(O)CN1CCC(n2c(=O)n(C3CCC(=O)NC3=O)c3ccccc32)CC1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)CN2CCC(n3c(=O)n(C4CCC(=O)NC4=O)c4ccccc43)CC2)c1]

### Step 1: 3-(carboxymethoxy)-4-chloro-5-[3-[[(4S)-1-[3-[2-[4-[3-(2,6-dioxo-3-piperidyl)-2-oxo-benzimidazol-1-yl]-1-piperidyl]acetyl]amino]phenyl]methylsulfonyl]-2,2-dimethyl-4-piperidyl]amino]phenyl]thiophene-2-carboxylic acid (130)

3-(carboxymethoxy)-4-chloro-5-[3-[[(4S)-1-[[3-[[2-[4-[3-(2,6-dioxo-3-piperidyl)-2-oxo-benzimidazol-1-yl]-1-piperidyl]acetyl]amino]phenyl]methylsulfonyl]-2,2-dimethyl-4-piperidyl]amino]phenyl]thiophene-2-carboxylic acid (130, 60 mg, 52.44 μmol, 21% yield, TFA salt) was synthesized from 2-[4-[3-(2,6-dioxo-3-piperidyl)-2-oxo-benzimidazol-1-yl]-1-piperidyl]acetic acid (B-311) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1 except using DMF as the solvent and heating the reaction to 85° C. The residue was subjected to reverse phase preparative-HPLC [Purification method: Column: XSelect C18 (150×19 mm) 5 μm; Mobile phase A: 0.1% TFA in water and Mobile phase B: MeCN]. LCMS (ES+): m/z 978.3 [M+H]⁺. ¹H NMR (400 MHz, DMSO-d6): δ 11.13 (s, 1H), 10.69 (s, 1H), 10.03 (s, 1H), 7.69-7.64 (m, 2H), 7.54 (d, J=7.20 Hz, 1H), 7.41 (t, J=7.60 Hz, 1H), 7.20-7.02 (m, 6H), 6.96 (s, 1H), 6.81 (d, J=7.60 Hz, 1H), 6.70 (d, J=8.40 Hz, 1H), 5.39 (dd, J=7.20, 13.00 Hz, 1H), 4.92 (s, 2H), 4.65-4.58 (m, 1H), 4.50-4.30 (m, 2H), 4.21 (s, 2H), 2.97-2.67 (m, 8H), 2.09-1.80 (m, 8H), 1.50 (s, 3H), 1.45-1.35 (m, 4H), 1.09-1.07 (m, 1H).

### Example 131

### 3-(carboxymethoxy)-4-chloro-5-[3-[(4R)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (131)

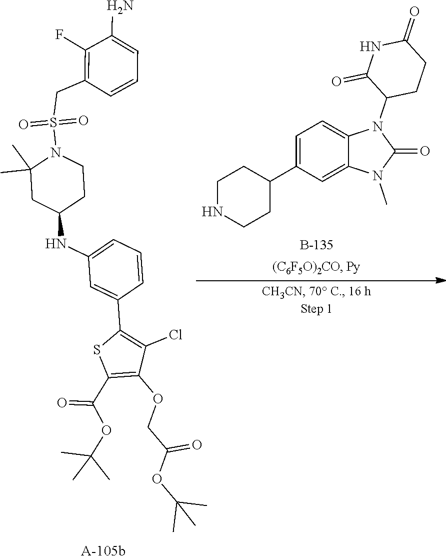 [SMILES: CC(C)(C)OC(=O)COc1c(C(=O)OC(C)(C)C)sc(-c2cccc(N[C@@H]3CCN(S(=O)(=O)Cc4cccc(N)c4F)C(C)(C)C3)c2)c1Cl ; Cn1c(=O)n(C2CCC(=O)NC2=O)c2ccc(C3CCNCC3)cc21]

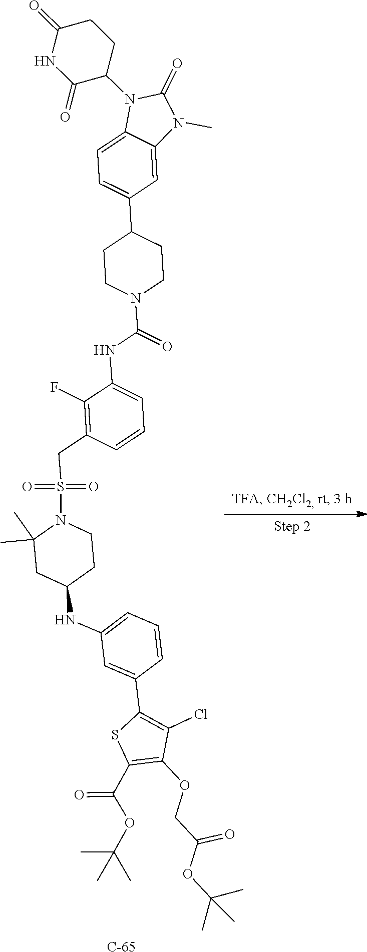

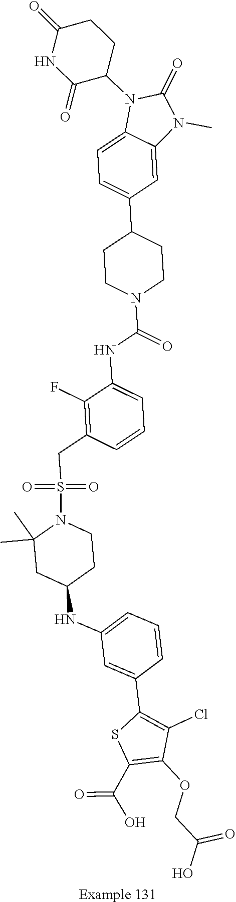 [SMILES: Cn1c(=O)n(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6)CC5(C)C)c4F)CC3)cc21 ; "Example 131"]

### Step 1: ter-butyl 3-(2-tert-butoxy-2-oxo-ethoxy)-4-chloro-5-[3-[(4R)-1-[3-[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylate (C-65)

Tert-butyl 3-(2-tert-butoxy-2-oxo-ethoxy)-4-chloro-5-[3-[(4R)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylate (C-65, 75 mg, 8.56 μmol, 9% yield) was synthesized from tert-butyl 5-[3-[[(4R)-1-[(3-amino-2-fluoro-phenyl)methylsulfonyl]-2,2-dimethyl-4-piperidyl]amino]phenyl]-3-(2-tert-butoxy-2-oxo-ethoxy)-4-chloro-thiophene-2-carboxylate (A-105b) and 3-[3-methyl-2-oxo-5-(4-piperidyl)benzimidazol-1-yl]piperidine-2,6-dione (B-135) in a similar fashion to Example 125. The crude compound was used in the next step. LCMS (ES+): m/z 1106.6 [M+H]⁺.

### Step 2: 3-(carboxymethoxy)-4-chloro-5-[3-[[(4R)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (131)

3-(carboxymethoxy)-4-chloro-5-[3-[(4R)-1-[[3-[[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylic acid (131, 4 mg, 3.55 μmol, 45% yield, TFA salt) was synthesized from tert-butyl 3-(2-tert-butoxy-2-oxo-ethoxy)-4-chloro-5-[3-[(4R)-1-[3-[4-[1-(2,6-dioxo-3-piperidyl)-3-methyl-2-oxo-benzimidazol-5-yl]piperidine-1-carbonyl]amino]-2-fluoro-phenyl]methylsulfonyl]-2,2-dimethyl-4-piperidyl]amino]phenyl]thiophene-2-carboxylate (C-65) in a similar fashion to Compound C-1, except using 222 eq. of TFA. The residue was purified by reversed phase flask chromatography [Purification method: RediSep ISCO C18 (30 g) column; Mobile phase A: 0.1% TFA in MQ-water; Mobile phase B: MeCN]. LCMS (ES+): m/z 994.5 [M+H]⁺. ¹H NMR (400 MHz, DMSO-d₆) δ 11.09 (s, 1H), 8.39 (s, 1H), 7.46-7.39 (m, 1H), 7.25-7.08 (m, 6H), 7.02 (d, J=8.1 Hz, 1H), 6.96 (s, 1H), 6.92 (d, J=8.1 Hz, 1H), 6.85 (s, 1H), 6.80 (d, J=7.6 Hz, 1H), 6.68 (d, J=8.3 Hz, 1H), 5.67 (s, 1H), 5.34 (dd, J=13.0, 5.3 Hz, 1H), 4.87 (s, 2H), 4.47 (d, J=14.0 Hz, 1H), 4.38 (d, J=13.9 Hz, 1H), 4.33-4.24 (m, 2H), 3.19-3.10 (m, 2H), 2.97-2.85 (m, 3H), 2.81-2.70 (m, 2H), 2.06-1.96 (m, 1H), 1.86-1.74 (m, 4H), 1.71-1.57 (m, 2H), 1.52 (s, 3H), 1.46-1.34 (m, 4H), 1.29-1.18 (m, 3H), 1.10-0.97 (m, 1H).

### Example 132

### 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-4-fluoro-phenyl]thiophene-2-carboxylic acid (132)

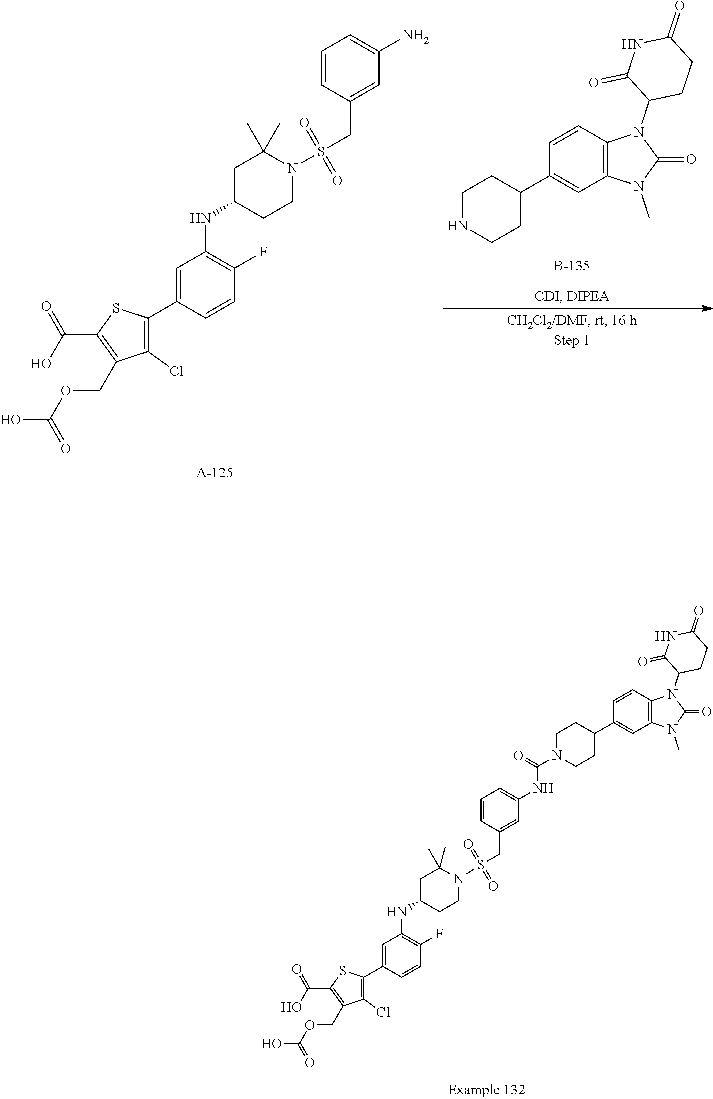 [SMILES: CC1(C)C[C@@H](Nc2cc(-c3sc(C(=O)O)c(COC(=O)O)c3Cl)ccc2F)CCN1S(=O)(=O)Cc1cccc(N)c1 ; Cn1c(=O)n(C2CCC(=O)NC2=O)c2ccc(C3CCNCC3)cc21 ; "A-125" ; Cn1c(=O)n(C2CCC(=O)NC2=O)c2ccc(C3CCN(C(=O)Nc4cccc(CS(=O)(=O)N5CC[C@H](Nc6cc(-c7sc(C(=O)O)c(COC(=O)O)c7Cl)ccc6F)CC5(C)C)c4)CC3)cc21]

### Step 1: 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-4-fluoro-phenyl]thiophene-2-carboxylic acid (132)

3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-4-fluoro-phenyl]thiophene-2-carboxylic acid (132, 49 mg, 42.76 μmol, 21% yield, TFA salt) was synthesized from 5-[3-[[(4S)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-125) and 3-[3-methyl-2-oxo-5-(4-piperidyl)benzimidazol-1-yl]piperidine-2,6-dione (B-135) in a similar fashion to Example 23 except using excess DIPEA as base and DCM/DMF as the solvent. The residue was purified by reverse-phase preparative-HPLC [Column: YMC-C18 (19×150 mm) 5 μm; Mobile phase A: 0.1% TFA in water and Mobile phase B: CH₃CN]. LCMS (ES−): m/z 993.5 [M−H]⁻. ¹H NMR (400 MHz, DMSO-d₆) δ 10.80 (d, J=7.9 Hz, 1H), 8.43 (d, J=13.0 Hz, 1H), 7.60 (d, J=2.3 Hz, 1H), 7.44 (dd, J=7.9, 2.4 Hz, 1H), 7.27-7.21 (m, 1H), 7.17-7.10 (m, 1H), 7.08-6.99 (m, 4H), 6.96-6.92 (m, 1H), 6.87-6.82 (m, 1H), 5.34-5.22 (m, 1H), 4.89 (s, 2H), 4.36-4.16 (m, 5H), 3.74-3.69 (m, 1H), 3.68-3.57 (m, 1H), 2.96-2.84 (m, 3H), 2.80-2.57 (m, 4H), 2.10-2.01 (m, 1H), 1.92-1.77 (m, 4H), 1.72-1.59 (m, 3H), 1.50 (s, 3H), 1.43-1.29 (m, 5H).

### Example 133

### 3-(carboxymethoxy)-4-chloro-5-[3-[(4R)-1-[3-[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-4-fluoro-phenyl]thiophene-2-carboxylic acid (133)

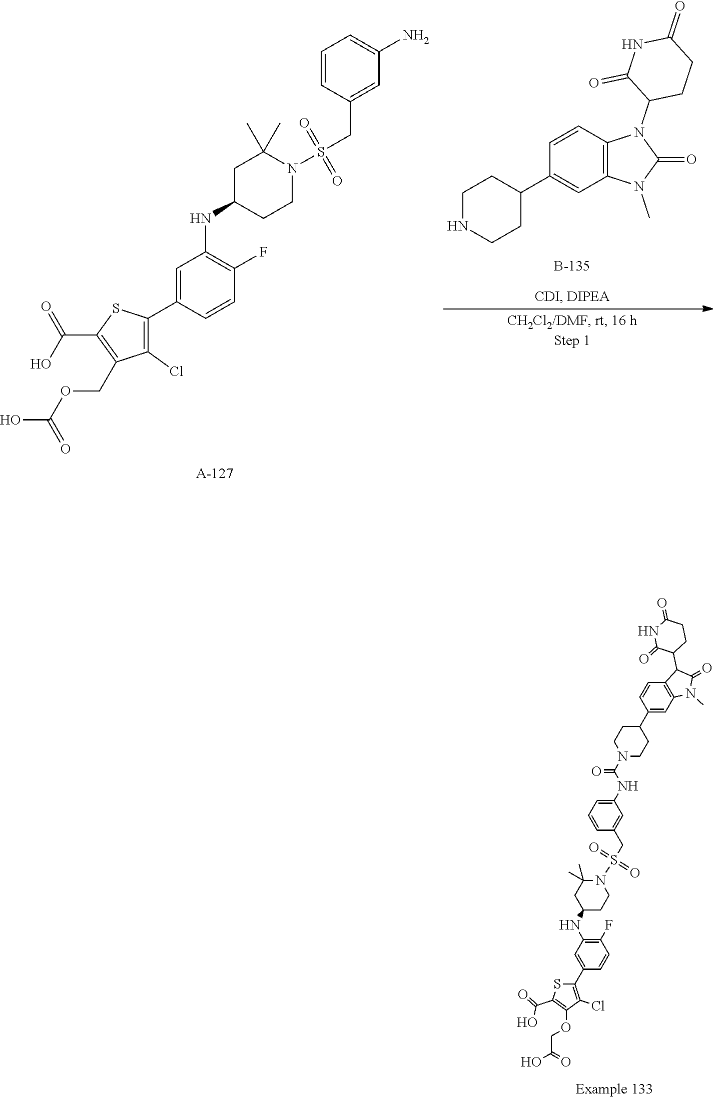 [SMILES: CC1(C)C[C@H](Nc2cc(-c3sc(C(=O)O)c(COC(=O)O)c3Cl)ccc2F)CCN1S(=O)(=O)Cc1cccc(N)c1 ; Cn1c(=O)n(C2CCC(=O)NC2=O)c2ccc(C3CCNCC3)cc21 ; "A-127" ; CN1C(=O)C(C2CCC(=O)NC2=O)c2ccc(C3CCN(C(=O)Nc4cccc(CS(=O)(=O)N5CC[C@@H](Nc6cc(-c7sc(C(=O)O)c(OCC(=O)O)c7Cl)ccc6F)CC5(C)C)c4)CC3)cc21]

### Step 1: 3-(carboxymethoxy)-4-chloro-5-[3-[(4R)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-4-fluoro-phenyl]thiophene-2-carboxylic acid (133)

3-(carboxymethoxy)-4-chloro-5-[3-[[(4R)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-4-fluoro-phenyl]thiophene-2-carboxylic acid (133, 35 mg, 30.15 μmol, 14% yield) was synthesized from 5-[3-[(4R)-1-[(3-aminophenyl)methylsulfonyl]-2,2-dimethyl-4-piperidyl]amino]-4-fluoro-phenyl]-3-(carboxymethoxy)-4-chloro-thiophene-2-carboxylic acid (A-127) and 3-[3-methyl-2-oxo-5-(4-piperidyl)benzimidazol-1-yl]piperidine-2,6-dione (B-135) in a similar fashion to Example 23 except using excess DIPEA as base and DCM/DMF as the solvent. The residue was purified by reverse phase preparatory HPLC [Purification method: Column: YMC C18 (150×19 mm), 5 μm, Mobile phase A: 0.1% TFA in water and Mobile phase B: MeCN]. ¹H NMR (400 MHz, DMSO-d₆) δ 10.81 (d, J=8.1 Hz, 1H), 8.42 (d, J=12.5 Hz, 1H), 7.61 (s, 1H), 7.48-7.41 (m, 1H), 7.28-7.22 (m, 1H), 7.17-7.10 (m, 1H), 7.08-6.98 (m, 4H), 6.96-6.92 (m, 1H), 6.88-6.82 (m, 1H), 5.34-5.23 (m, 1H), 4.89 (s, 2H), 4.36-4.16 (m, 5H), 3.72 (t, J=5.7 Hz, 1H), 3.67-3.59 (m, 2H), 2.96-2.88 (m, 2H), 2.74-2.62 (m, 2H), 2.12-2.01 (m, 1H), 1.93-1.77 (m, 4H), 1.71-1.60 (m, 2H), 1.50 (s, 3H), 1.43-1.29 (m, 4H). LCMS (ES+): m/z 994.2 [M+H]⁺.

### Example 134

### 3-(carboxymethoxy)-4-chloro-5-[3-[[(4S)-1-[[3-[[4-[1-(2,6-dioxo-3-piperidyl)-3-methyl-2-oxo-benzimidazol-S-yl]piperidine-1-carbonyl]amino]phenyl]methylsulfonyl]-2,2-dimethyl-4-piperidyl]amino]-2-fluoro-phenyl]thiophene-2-carboxylic acid (134)

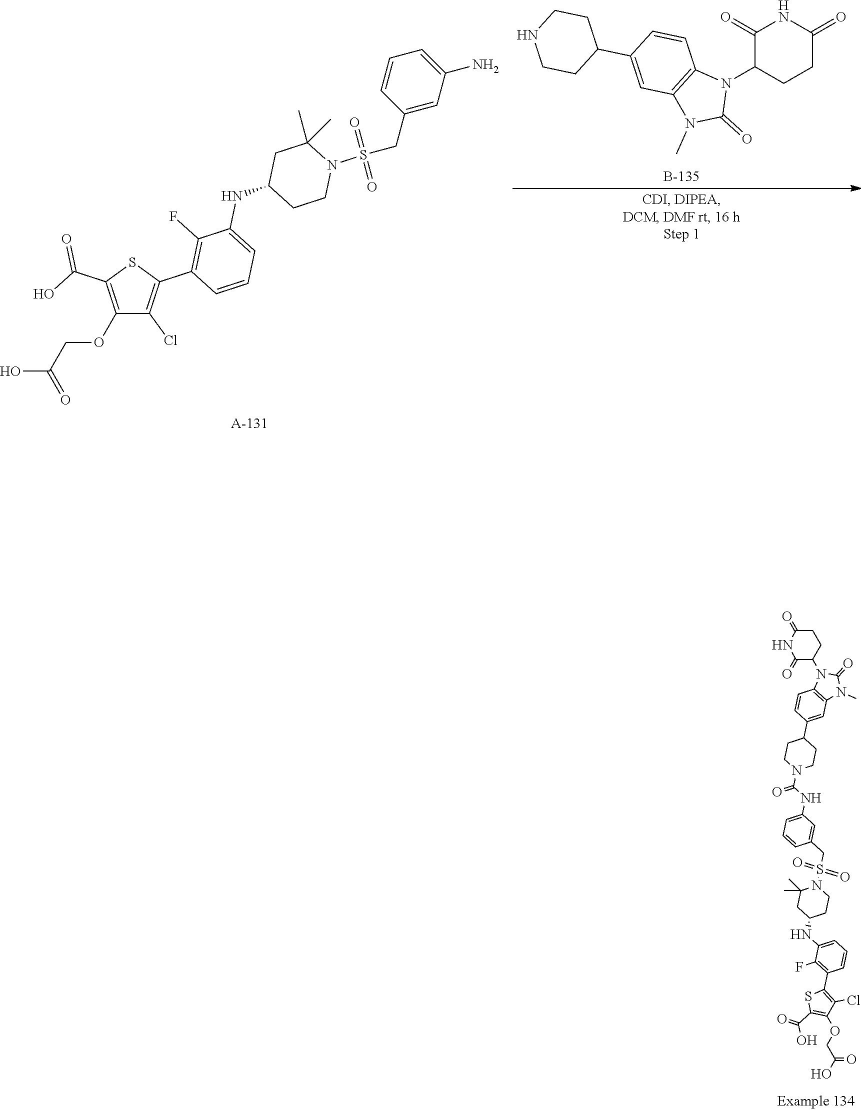 [SMILES: CC1(C)C[C@@H](Nc2cccc(-c3sc(C(=O)O)c(OCC(=O)O)c3Cl)c2F)CCN1S(=O)(=O)Cc1cccc(N)c1 ; Cn1c(=O)n(C2CCC(=O)NC2=O)c2ccc(C3CCNCC3)cc21 ; "A-131" ; Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6F)CC5(C)C)c4)CC3)cc21]

### Step 1: 3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-2-fluoro-phenyl]thiophene-2-carboxylic acid (134)

3-(carboxymethoxy)-4-chloro-5-[3-[[(4S)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-2-fluoro-phenyl]thiophene-2-carboxylic acid (134, 38 mg, 32.82 μmol, 15% yield, TEA salt) was synthesized from 5-[3-[[(4S)-1-[(3-aminophenyl)methylsulfonyl]-2,2-dimethyl-4-piperidyl]amino]-2-fluoro-phenyl]-3-(carboxymethoxy)-4-chloro-thiophene-2-carboxylic acid (A-131) and 3-[3-methyl-2-oxo-5-(4-piperidyl)benzimidazol-1-yl]piperidine-2,6-dione (B-135) in a similar fashion to Example 23 except using excess DIPEA as base and DCM/DMF as the solvent. The residue was purified by reverse phase preparatory HPLC [Purification method: Column: YMC C18 (250×19 mm), 5 μm, Mobile phase A: 0.1% TFA in water and Mobile phase B: MeCN]. LCMS (ES+): m/z 994.5 [M+H]⁺. ¹H NMR (400 MHz, DMSO-d₆) δ 10.80 (d, J=7.6 Hz, 1H), 8.42 (d, J=12.3 Hz, 1H), 7.60 (s, 1H), 7.46-7.40 (m, 1H), 7.28-7.21 (m, 2H), 7.13-6.97 (m, 5H), 6.95-6.89 (m, 2H), 6.69-6.64 (m, 1H), 5.32-5.22 (m, 1H), 4.87 (d, J=2.8 Hz, 2H), 4.36-4.13 (m, 5H), 3.71 (t, 2H), 3.18-3.10 (m, 2H), 2.95-2.84 (m, 2H), 2.82-2.63 (m, 3H), 2.61-2.54 (m, 1H), 2.13-2.01 (m, 1H), 1.91-1.59 (m, 6H), 1.50 (d, J=2.1 Hz, 3H), 1.41 (s, 3H), 1.34-1.22 (m, 1H).

### Example 135

### 3-(carboxymethoxy)-4-chloro-5-[3-[[(4R)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-2-fluoro-phenyl]thiophene-2-carboxylic acid (135)

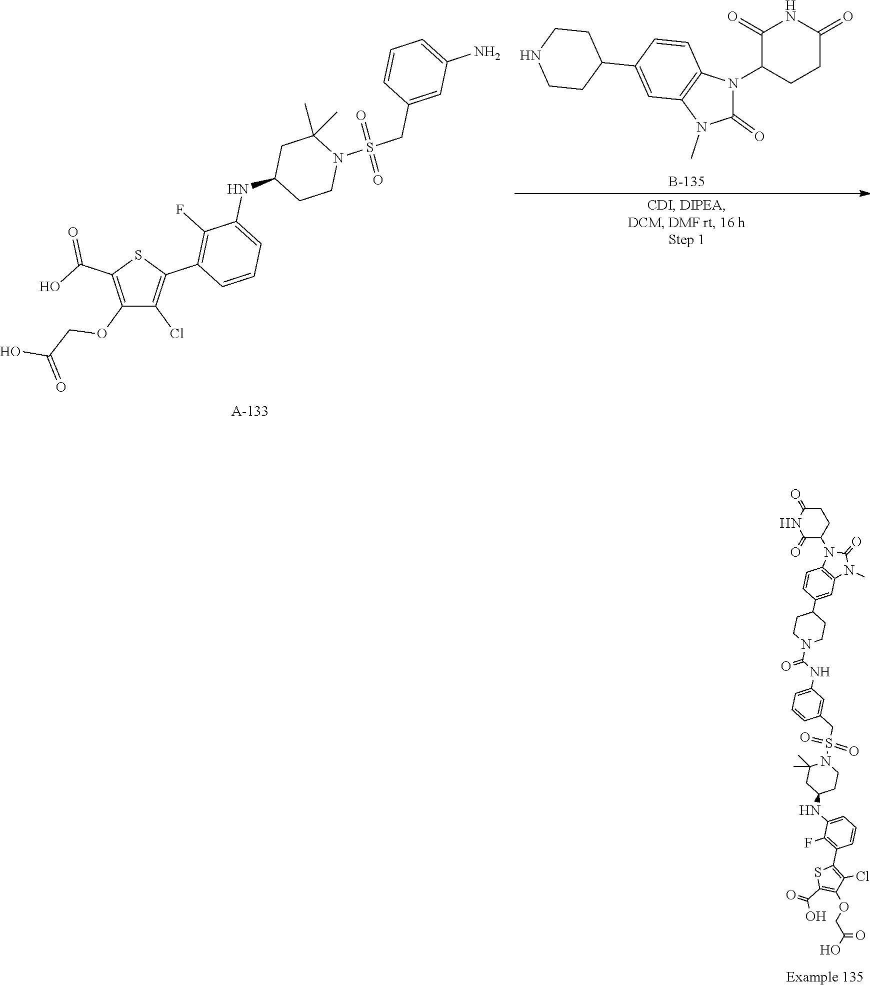 [SMILES: CC1(C)C[C@H](Nc2cccc(-c3sc(C(=O)O)c(OCC(=O)O)c3Cl)c2F)CCN1S(=O)(=O)Cc1cccc(N)c1 ; Cn1c(=O)n(C2CCC(=O)NC2=O)c2ccc(C3CCNCC3)cc21 ; "A-133" ; Cn1c(=O)n(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6F)CC5(C)C)c4)CC3)cc21]

### Step 1: 3-(carboxymethoxy)-4-chloro-5-[3-[[(4R)-1-[[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-2-fluoro-phenyl]thiophene-2-carboxylic acid (135)

3-(carboxymethoxy)-4-chloro-5-[3-[(4R)-1-[3-[[4-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]-2-fluoro-phenyl]thiophene-2-carboxylic acid (135, 45 mg, 40.4 μmol, 18% yield) was synthesized from 2-[5-[3-[[(4R)-1-[(3-aminophenyl)methylsulfonyl]-2,2-dimethyl-4-piperidyl]amino]-2-fluoro-phenyl]-2-tert-butoxycarbonyl-4-chloro-3-thienyl]oxy]acetic acid (A-133) and 3-[3-methyl-2-oxo-5-(4-piperidyl)benzimidazol-1-yl]piperidine-2,6-dione (B-135) in a similar fashion to Example 23 except using excess DIPEA as base and DCM/DMF as the solvent. The residue was purified by reverse phase preparatory HPLC [Purification method: Column: YMC C18 (250×19 mm), 5 μm, Mobile phase A: 0.1% TFA in water and Mobile phase B: MeCN]. LCMS (ES+): m/z 994.2 [M+H]⁺. ¹H NMR (400 MHz, DMSO-d₆) δ 10.80 (d, J=7.6 Hz, 1H), 8.43 (d, J=12.4 Hz, 1H), 7.66-7.60 (m, 1H), 7.47-7.43 (m, 1H), 7.28-7.21 (m, 2H), 7.12-6.97 (m, 4H), 6.96-6.89 (m, 1H), 6.70-6.64 (m, 1H), 5.33-5.23 (m, 1H), 4.88 (d, J=2.3 Hz, 2H), 4.36-4.16 (m, 5H), 3.72 (t, J=5.5 Hz, 1H), 3.66-3.59 (m, 2H), 3.51-3.44 (m, 2H), 2.96-2.86 (m, 2H), 2.74-2.63 (m, 2H), 2.11-2.03 (m, 1H), 1.92-1.74 (m, 3H), 1.72-1.60 (m, 2H), 1.51 (d, J=1.9 Hz, 3H), 1.42 (s, 3H), 1.37-1.23 (m, 1H).

### Example 136

### 3-(carboxymethoxy)-4-chloro-5-[3-[[(4S)-1-[3-[[1-[1-(2,6-dioxo-3-piperidyl)-3-methyl-2-oxo-benzimidazol-5-yl]azetidin-3-yl]oxycarbonylamino]phenyl]methylsulfonyl]-2,2-dimethyl-4-piperidyl]amino]phenyl]thiophene-2-carboxylic acid (136)

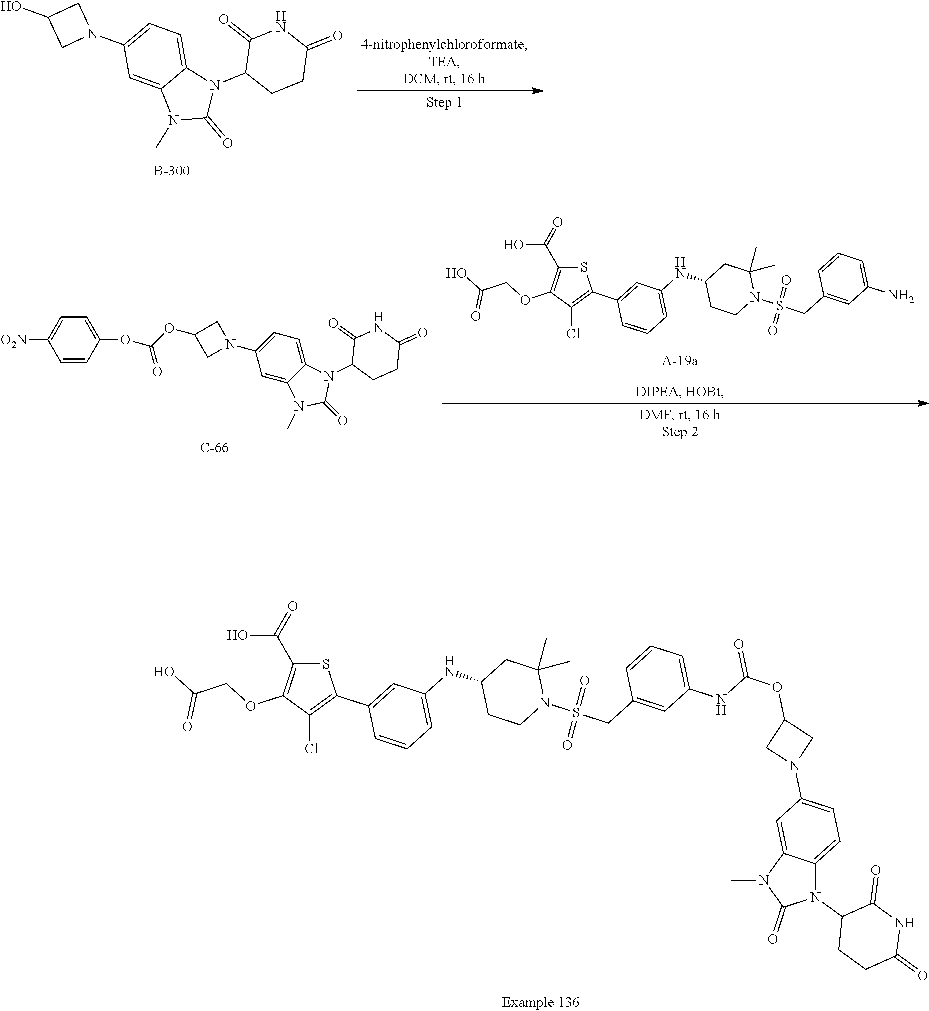

### Step 1; [1-[1-(2,6-dioxo-3-piperidyl)-3-methyl-2-oxo-benzimidazol-5-yl]azetidin-3-yl](4-nitrophenyl)carbonate (C-66)

[1-[1-(2,6-dioxo-3-piperidyl)-3-methyl-2-oxo-benzimidazol-5-yl]azetidin-3-yl](4-nitrophenyl)carbonate (C-66, 200 mg, 141.29 μmol, 44% yield) was synthesized from 3-[5-(3-hydroxyazetidin-1-yl)-3-methyl-2-oxo-benzimidazol-1-yl]piperidine-2,6-dione (B-300) in a similar fashion to Compound C-42. The crude compound was used in the next step without purification. LCMS (ES+): m/z 495.9 [M+H]⁺.

### Step 2: 3-(carboxymethoxy)-4-chloro-5-[3-[[(4S)-1-[[3-[1-[1-(2,6-dioxo-3-piperidyl)-3-methyl-2-oxo-benzimidazol-5-yl]azetidin-3-yl]oxycarbonylamino]phenyl]methylsulfonyl]-2,2-dimethyl-4-piperidyl]amino]phenyl]thiophene-2-carboxylic acid (136)

3-(carboxymethoxy)-4-chloro-5-[3-[[(4S)-1-[3-[[1-[1-(2,6-dioxo-3-piperidyl)-3-methyl-2-oxo-benzimidazol-5-yl]azetidin-3-yl]oxycarbonylamino]phenyl]methylsulfonyl]-2,2-dimethyl-4-piperidyl]amino]phenyl]thiophene-2-carboxylic acid (136, 4 mg, 2.99 μmol, 2% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and [1-[1-(2,6-dioxo-3-piperidyl)-3-methyl-2-oxo-benzimidazol-5-yl]azetidin-3-yl](4-nitrophenyl)carbonate (C-66) in a similar fashion to Example 85. The residue was purified by reverse phase prep HPLC [Purification method: Column: X-Bridge C8 (150×19 mm), 5 μm, Mobile phase A: 0.1% TFA in water, Mobile phase B: Acetonitrile]. LCMS (ES+): m/z 964.0 [M+H]⁺. ¹H NMR (400 MHz, DMSO-d6): δ 11.10 (s, 1H), 10.00 (s, 1H), 7.60 (s, 1H), 7.44 (d, J=8.40 Hz, 1H), 7.34-7.33 (m, 1H), 7.19-7.29 (m, 2H), 7.12 (s, 1H), 7.07-7.04 (m, 1H), 6.99 (s, 1H), 6.93 (d, J=8.40 Hz, 1H), 6.87 (s, 1H), 6.85-6.79 (m, 1H), 6.71-6.65 (m, 1H), 6.38 (d, J=11.20 Hz, 1H), 6.17 (dd, J=2.00, 8.40 Hz, 1H), 5.73 (s, 1H), 5.31-5.26 (m, 2H), 4.90 (s, 2H), 4.34 (dd, J=13.60, 47.80 Hz, 2H), 4.22-4.18 (m, 2H), 3.77-3.74 (m, 2H), 3.10-3.09 (m, 2H), 1.80-1.98 (m, 3H), 1.46-1.39 (m, 8H), 1.15-1.05 (m, 1H).

### Example 137

### 3-(carboxymethoxy)-4-chloro-5-[3-[(4R)-1-[3-[4-[1-(2,6-dioxo-3-piperidyl)-3-isopropyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (137)

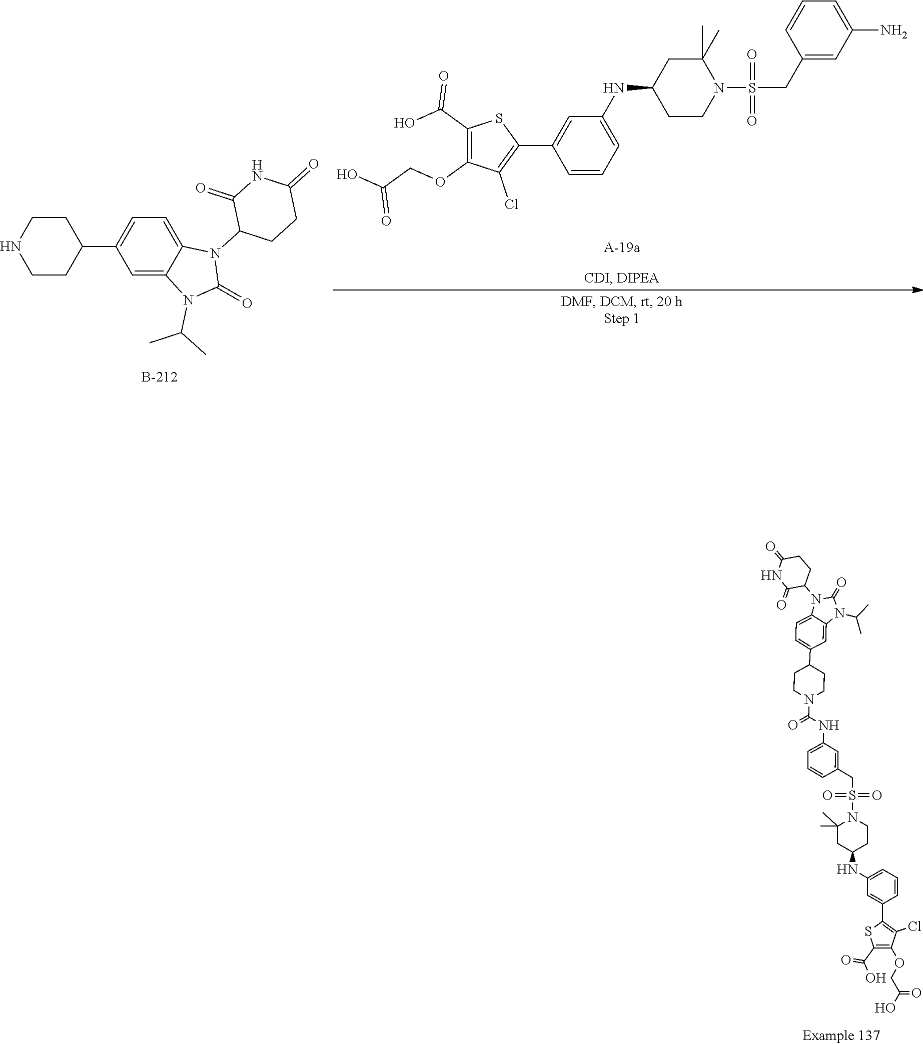 [SMILES: CC(C)n1c(=O)n(C2CCC(=O)NC2=O)c2ccc(C3CCNCC3)cc21 ; CC1(C)C[C@H](Nc2cccc(-c3sc(C(=O)O)c(OCC(=O)O)c3Cl)c2)CCN1S(=O)(=O)Cc1cccc(N)c1 ; CC(C)n1c(=O)n(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4R)-1-[[3-[[4-[1-(2,6-dioxo-3-piperidyl)-3-isopropyl-2-oxo-benzimidazol-S-yl]piperidine-1-carbonyl]amino]phenyl]methylsulfonyl]-2,2-dimethyl-4-piperidyl]amino]phenyl]thiophene-2-carboxylic acid (137)

3-(carboxymethoxy)-4-chloro-5-[3-[[(4R)-1-[[3-[[4-[1-(2,6-dioxo-3-piperidyl)-3-isopropyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (137, 35 mg, 30.59 μmol, 6% yield, TFA salt) was synthesized from 5-[3-[(4R)-1-[(3-aminophenyl)methylsulfonyl]-2,2-dimethyl-4-piperidyl]amino]phenyl]-3-(carboxymethoxy)-4-chloro-thiophene-2-carboxylic acid (A-19b) and 3-[3-isopropyl-2-oxo-5-(4-piperidyl)benzimidazol-1-yl]piperidine-2,6-dione (B-212) in a similar fashion to Example 23 except using excess DIPEA as base and DCM/DMF as the solvent. The residue was purified by reverse phase prep HPLC [Purification method: Column: Zorbax prep HT SB-C18; Mobile phase A: 0.1% TFA in water; Mobile phase B: MeCN; Flow Rate: 15.0 mL/min]. LCMS (ES+): m/z 1004.3 [M+H]⁺. ¹H NMR (400 MHz, DMSO-d₆) δ 11.08 (s, 1H), 8.61 (s, 1H), 7.55 (s, 1H), 7.48-7.41 (m, 1H), 7.27-7.16 (m, 3H), 6.99 (t, J=7.5 Hz, 2H), 6.90 (d, J=8.2 Hz, 1H), 6.87-6.84 (m, 1H), 6.80 (d, J=7.6 Hz, 1H), 6.71-6.66 (m, 1H), 5.29 (dd, J=12.8, 5.4 Hz, 1H), 4.89 (s, 2H), 4.59 (p, J=6.9 Hz, 1H), 4.40-4.17 (m, 4H), 3.16-3.06 (m, 2H), 2.95-2.83 (m, 3H), 2.81-2.71 (m, 2H), 2.70-2.57 (m, 2H), 2.03-1.94 (m, 1H), 1.88-1.72 (m, 4H), 1.69-1.55 (m, 2H), 1.47 (s, 3H), 1.45 (s, 3H), 1.43 (s, 3H), 1.38 (s, 3H), 1.16-1.00 (m, 1H).

### Example 138

### 3-(carboxymethoxy)-4-chloro-5-(3-(((4S)-1-((3-((1r,4S)-4-(1-(2,6-dioxopiperidin-3-yl)-3-methyl-2-oxo-2,3-dihydro-1H-benzo[d]imidazol-5-yl)cyclohexane-1-carboxamido)benzyl)sulfonyl)-2,2-dimethylpiperidin-4-yl)amino)phenyl)thiophene-2-carboxylic acid (138)

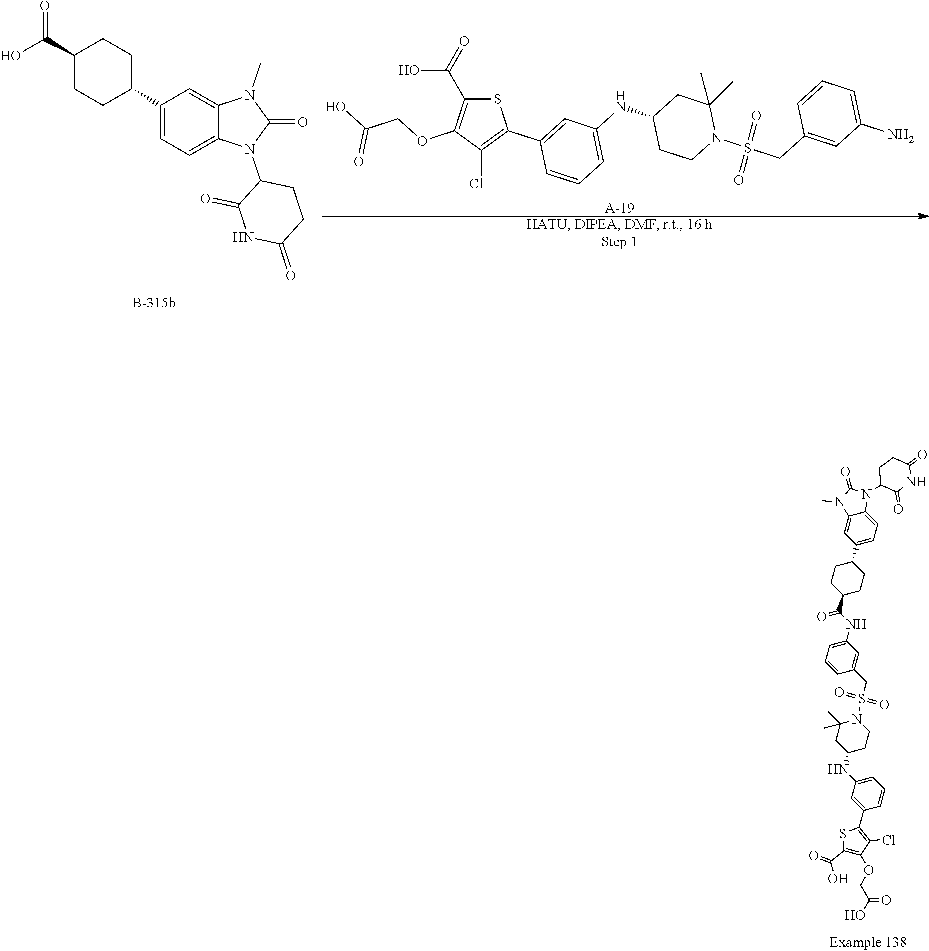

### Step 1: 3-(carboxymethoxy)-4-chloro-5-(3-(((4S)-1-((3-((1r,4S)-4-(1-(2,6-dioxopiperidin-3-yl)-3-methyl-2-oxo-2,3-dihydro-1H-benzo[d]imidazol-5-yl)cyclohexane-1-carboxamido)benzyl)sulfonyl)-2,2-dimethylpiperidin-4-yl)amino)phenyl)thiophene-2-carboxylic acid (138)

3-(carboxymethoxy)-4-chloro-5-(3-(((4S)-1-((3-((1r,4S)-4-(1-(2,6-dioxopiperidin-3-yl)-3-methyl-2-oxo-2,3-dihydro-1H-benzo[d]imidazol-5-yl)cyclohexane-1-carboxamido)benzyl)sulfonyl)-2,2-dimethylpiperidin-4-yl)amino)phenyl)thiophene-2-carboxylic acid (138, 6.5 mg, 6.50 μmol, 5% yield) was synthesized from (1r,4r)-4-(1-(2,6-dioxopiperidin-3-yl)-3-methyl-2-oxo-2,3-dihydro-1H-benzo[d]imidazol-5-yl)cyclohexane-1-carboxylic acid (B-315b) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1 except using DMF as the solvent. The residue was purified by reverse phase prep HPLC [Purification method: Column: XSelect C18 (10×150 mm) 5 μm, Mobile phase A: 10 mM ammonium acetate in MQ water, Mobile phase B: Acetonitrile]. LCMS (ES+): m/z 975.3 [M+H]⁺. ¹H NMR (400 MHz, DMSO-d₆) δ 11.08 (s, 1H), 9.86 (s, 1H), 7.70-7.67 (m, 1H), 7.67-7.62 (m, 1H), 7.30 (t, J=7.9 Hz, 1H), 7.22-7.13 (m, 2H), 7.07 (d, J=7.7 Hz, 1H), 7.02-6.97 (m, 2H), 6.89 (d, J=8.2 Hz, 1H), 6.84-6.81 (m, 1H), 6.76 (d, J=7.5 Hz, 1H), 6.67-6.61 (m, 1H), 5.62 (d, J=7.8 Hz, 1H), 5.32 (dd, J=12.8, 5.3 Hz, 1H), 4.67 (s, 2H), 4.39 (d, J=13.7 Hz, 1H), 4.29 (d, J=13.7 Hz, 1H), 3.58-3.49 (m, 2H), 3.17-3.08 (m, 2H), 2.96-2.84 (m, 1H), 2.76-2.60 (m, 4H), 2.13-1.84 (m, 6H), 1.82-1.75 (m, 1H), 1.74-1.63 (m, 4H), 1.47 (s, 3H), 1.44-1.37 (m, 4H), 1.29-1.23 (m, 1H), 1.17-1.03 (m, 1H).

### Example 139

### 3-(carboxymethoxy)-4-chloro-5-(3-(((4S)-1-((3-((1s,4R)-4-(1-(2,6-dioxopiperidin-3-yl)-3-methyl-2-oxo-2,3-dihydro-1H-benzo[d]imidazol-5-yl)cyclohexane-1-carboxamido)benzyl)sulfonyl)-2,2-dimethylpiperidin-4-yl)amino)phenyl)thiophene-2-carboxylic acid (139)

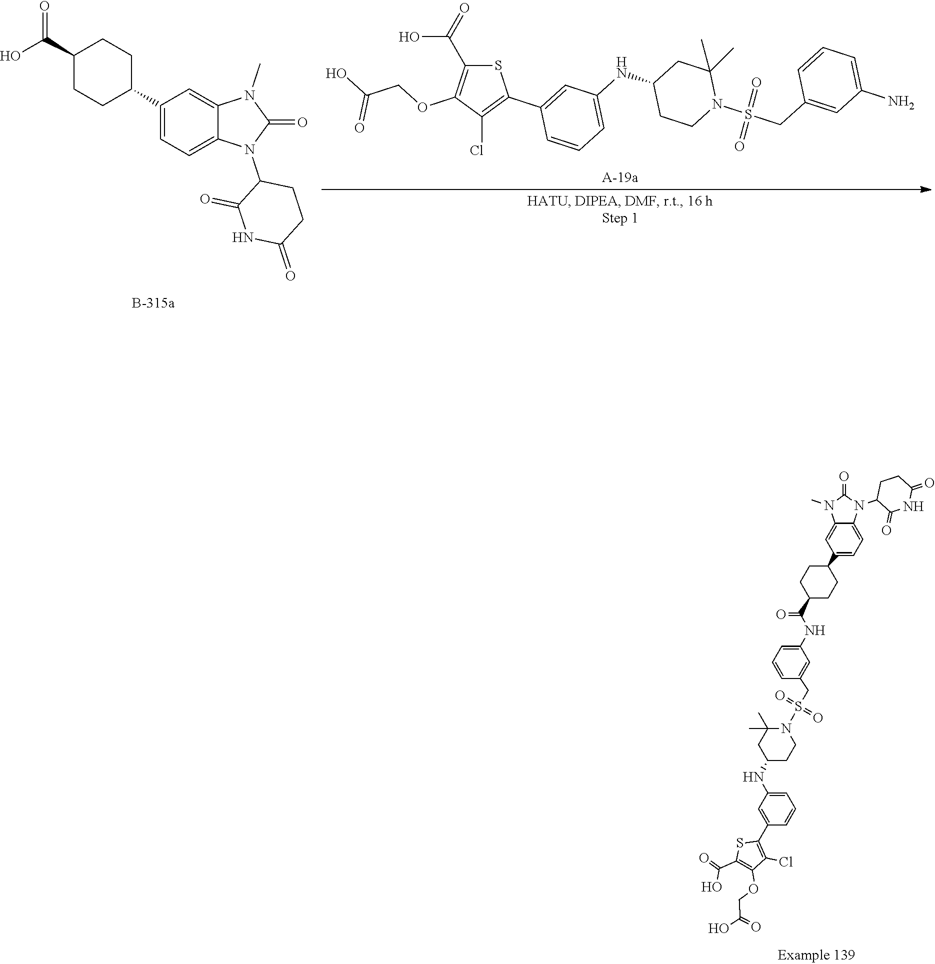 [SMILES: Cn1c(=O)n(C2CCC(=O)NC2=O)c2ccc([C@H]3CC[C@H](C(=O)O)CC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc([C@H]3CC[C@@H](C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-(3-(((4S)-1-((3-((1s,4R)-4-(1-(2,6-dioxopiperidin-3-yl)-3-methyl-2-oxo-2,3-dihydro-1H-benzo[d]imidazol-5-yl)cyclohexane-1-carboxamido)benzyl)sulfonyl)-2,2-dimethylpiperidin-4-yl)amino)phenyl)thiophene-2-carboxylic acid (139)

3-(carboxymethoxy)-4-chloro-5-(3-(((4S)-1-((3-((1s,4R)-4-(1-(2,6-dioxopiperidin-3-yl)-3-methyl-2-oxo-2,3-dihydro-1H-benzo[d]imidazol-5-yl)cyclohexane-1-carboxamido)benzyl)sulfonyl)-2,2-dimethylpiperidin-4-yl)amino)phenyl)thiophene-2-carboxylic acid (139, 8 mg, 8.04 μmol, 6% yield) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and (1s,4s)-4-(1-(2,6-dioxopiperidin-3-yl)-3-methyl-2-oxo-2,3-dihydro-1H-benzo[d]imidazol-5-yl)cyclohexane-1-carboxylic acid (B-315a) in a similar fashion to Example 1 except using DMF as the solvent. The residue was purified by reverse phase preparatory HPLC [Purification method: Column: XSelect C18 (150×10 mm), 5 μm, Mobile phase A: 0.1% TFA in water and Mobile phase B: MeCN]. LCMS (ES−): m/z 973.2 [M−H]⁻. ¹H NMR (400 MHz, DMSO-d₆) δ 11.08 (s, 1H), 9.96 (s, 1H), 7.73 (s, 1H), 7.63-7.58 (m, 1H), 7.30 (t, J=7.9 Hz, 1H), 7.22-7.15 (m, 1H), 7.10-7.05 (m, 2H), 7.01 (d, J=8.1 Hz, 1H), 6.96-6.89 (m, 1H), 6.87-6.83 (m, 1H), 6.78 (d, J=7.8 Hz, 1H), 6.70-6.63 (m, 1H), 5.61 (d, J=7.7 Hz, 1H), 5.33 (dd, J=12.7, 5.5 Hz, 1H), 4.67 (s, 2H), 4.39 (d, J=13.7 Hz, 1H), 4.29 (d, J=13.7 Hz, 1H), 3.57-3.49 (m, 2H), 3.16-3.08 (m, 2H), 2.96-2.85 (m, 1H), 2.74-2.57 (m, 3H), 2.04-1.85 (m, 6H), 1.83-1.76 (m, 1H), 1.71-1.51 (m, 4H), 1.47 (s, 3H), 1.43-1.36 (m, 4H), 1.19-1.05 (m, 1H).

### Example 140

### 3-(carboxymethoxy)-4-chloro-5-[3-[[(4S)-1-[[3-[[6-[1-(2,6-dioxo-3-piperidyl)-3-methyl-2-oxo-benzimidazol-5-yl]-2,6-diazaspiro[3.3]heptane-2-carbonyl]amino]phenyl]methylsulfonyl]-2,2-dimethyl-4-piperidyl]amino]phenyl]thiophene-2-carboxylic acid (140)

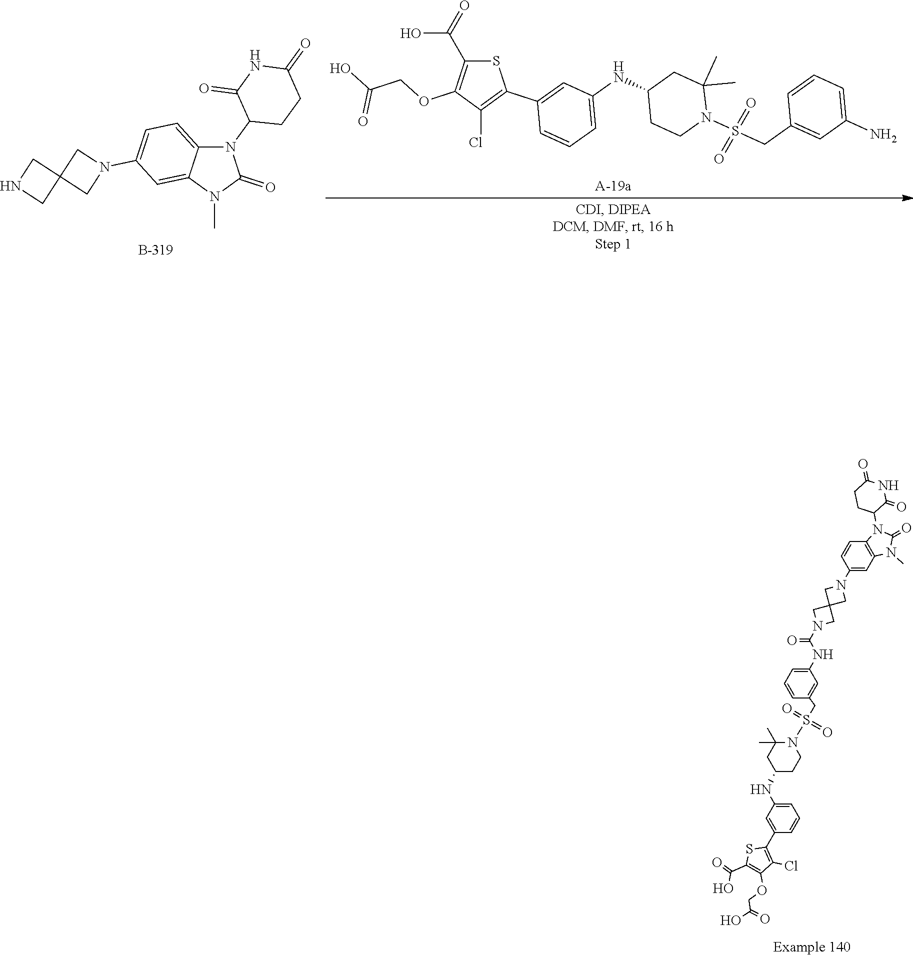 [SMILES: Cn1c(=O)n(C2CCC(=O)NC2=O)c2ccc(N3CC4(CNC4)C3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(N3CC4(CN(C(=O)Nc5cccc(CS(=O)(=O)N6CC[C@H](Nc7cccc(-c8sc(C(=O)O)c(OCC(=O)O)c8Cl)c7)CC6(C)C)c5)C4)C3)cc21]

### Step 1: 3-(carboxymethoxy)-4-chloro-5-[3-[[(4S)-1-[[3-[[6-[1-(2,6-dioxo-3-piperidyl)-3-methyl-2-oxo-benzimidazol-5-yl]-2,6-diazaspiro[3.3]heptane-2-carbonyl]amino]phenyl]methylsulfonyl]-2,2-dimethyl-4-piperidyl]amino]phenyl]thiophene-2-carboxylic acid (140)

3-(carboxymethoxy)-4-chloro-5-[3-[[(4S)-1-[3-[6-[1-(2,6-dioxo-3-piperidyl)-3-methyl-2-oxo-benzimidazol-5-yl]-2,6-diazaspiro[3.3]heptane-2-carbonyl]amino]phenyl]methylsulfonyl]-2,2-dimethyl-4-piperidyl]amino]phenyl]thiophene-2-carboxylic acid (140, 25.3 mg, 19.91 μmol, 7% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(2,6-diazaspiro[3.3]heptan-2-yl)-3-methyl-2-oxo-benzimidazol-1-yl]piperidine-2,6-dione (B-319) in a similar fashion to Example 23, except using excess DIPEA as base and DCM:DMF as the solvent. The residue was purified by reverse phase preparatory HPLC [Purification method: Column: Zorbax C18 (250×21.2 mm), 7 μm, Mobile phase A: 0.1% TFA in water and Mobile phase B: MeCN], LCMS (ES+): m/z 989.2 [M+H]⁺. ¹H NMR (400 MHz, DMSO-d6): δ 13.30 (bs, 1H), 11.06 (s, 1H), 8.60 (s, 1H), 7.59 (s, 1H), 7.52 (d, J=8.00 Hz, 1H), 7.27-7.19 (m, 2H), 7.00-6.92 (m, 2H), 6.88 (s, 1H), 6.82 (d, J=7.60 Hz, 1H), 6.71-6.69 (m, 1H), 6.36 (d, J=2.00 Hz, 1H), 6.15 (dd, J=2.00, 8.60 Hz, 1H), 5.29-5.26 (m, 1H), 4.92 (s, 2H), 4.38-4.23 (m, 2H), 4.16 (s, 3H), 3.94 (s, 3H), 3.54-3.43 (m, 3H), 3.30 (s, 3H), 3.15-3.08 (m, 1H), 2.95-2.82 (m, 1H), 2.66-2.60 (m, 2H), 2.04-1.92 (m, 1H), 1.88-1.76 (m, 2H), 1.47 (s, 3H), 1.39 (s, 3H), 1.13-1.09 (m, 1H).

### Example 141

### 3-(carboxymethoxy)-4-chloro-5-[3-[[(4S)-1-[3-[8-[1-(2,6-dioxo-3-piperidyl)-3-methyl-2-oxo-benzimidazol-5-yl]-2,8-diazaspiro[3.5]nonane-2-carbonyl]amino]phenyl]methylsulfonyl]-2,2-dimethyl-4-piperidyl]amino]phenyl]thiophene-2-carboxylic acid (141)

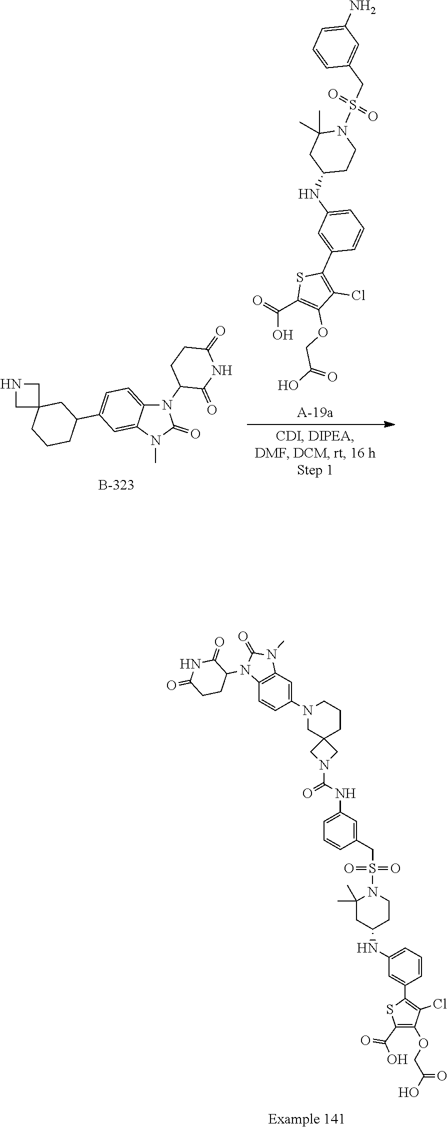 [SMILES: Cn1c(=O)n(C2CCC(=O)NC2=O)c2ccc(C3CCCC4(CNC4)C3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(N3CCCC4(CN(C(=O)Nc5cccc(CS(=O)(=O)N6CC[C@H](Nc7cccc(-c8sc(C(=O)O)c(OCC(=O)O)c8Cl)c7)CC6(C)C)c5)C4)C3)cc21]

### Step 1: 3-(carboxymethoxy)-4-chloro-5-[3-[[(4S)-1-[[3-[[8-[1-(2,6-dioxo-3-piperidyl)-3-methyl-2-oxo-benzimidazol-5-yl]-2,8-diazaspiro[3.5]nonane-2-carbonyl]amino]phenyl]methylsulfonyl]-2,2-dimethyl-4-piperidyl]amino]phenyl]thiophene-2-carboxylic acid (141)

3-(carboxymethoxy)-4-chloro-5-[3-[[(4S)-1-[3-[8-[1-(2,6-dioxo-3-piperidyl)-3-methyl-2-oxo-benzimidazol-5-yl]-2,8-diazaspiro[3.5]nonane-2-carbonyl]amino]phenyl]methylsulfonyl]-2,2-dimethyl-4-piperidyl]amino]phenyl]thiophene-2-carboxylic acid (141, 3 mg, 2.68 μmol, 2% yield, formate salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(2,6-diazaspiro[3.5]nonan-6-yl)-3-methyl-2-oxo-benzimidazol-1-yl]piperidine-2,6-dione (B-323) in a similar fashion to Example 23, except using excess DIPEA as base and DCM:DMF as the solvent. The residue was purified by reverse phase preparatory HPLC [Purification method: Column: Zorbax C18 (250×21.2 mm), 7 μm, Mobile phase A: 0.1% formic acid in water and Mobile phase B: MeCN]. LCMS (ES+): m/z 1017.2 [M+H]⁺. ¹H NMR (400 MHz, DMSO-d6): δ 11.07 (s, 1H), 8.49 (s, 1H), 7.60 (s, 1H), 7.51 (d, J=8.00 Hz, 1H), 7.24 (t, J=8.00 Hz, 1H), 7.17 (t, J=8.40 Hz, 1H), 6.98-6.91 (m, 3H), 6.83 (s, 1H), 6.78 (d, J=7.60 Hz, 1H), 6.70-6.65 (m, 2H), 6.54 (s, 1H), 5.63 (d, J=7.60 Hz, 1H), 5.31-5.28 (m, 1H), 4.69 (s, 2H), 4.29 (dd, J=13.60, 46.20 Hz, 2H), 3.79-3.48 (m, 6H), 3.17-3.09 (m, 4H), 2.98-2.90 (m, 4H), 2.69-2.64 (m, 2H), 1.69-1.99 (m, 8H), 1.45 (s, 3H), 1.39 (s, 3H), 1.24-1.11 (m, 2H).

### Example 142

### 3-(carboxymethoxy)-4-chloro-5-(3-((1-((3-(3-(1-(1-(2,6-dioxopiperidin-3-yl)-3-methyl-2-oxo-2,3-dihydro-1H-benzo[d]imidazol-5-yl)piperidin-4-yl)-2-oxoimidazolidin-1-yl)benzyl)sulfonyl)piperidin-4-yl)amino)phenyl)thiophene-2-carboxylic acid (142)

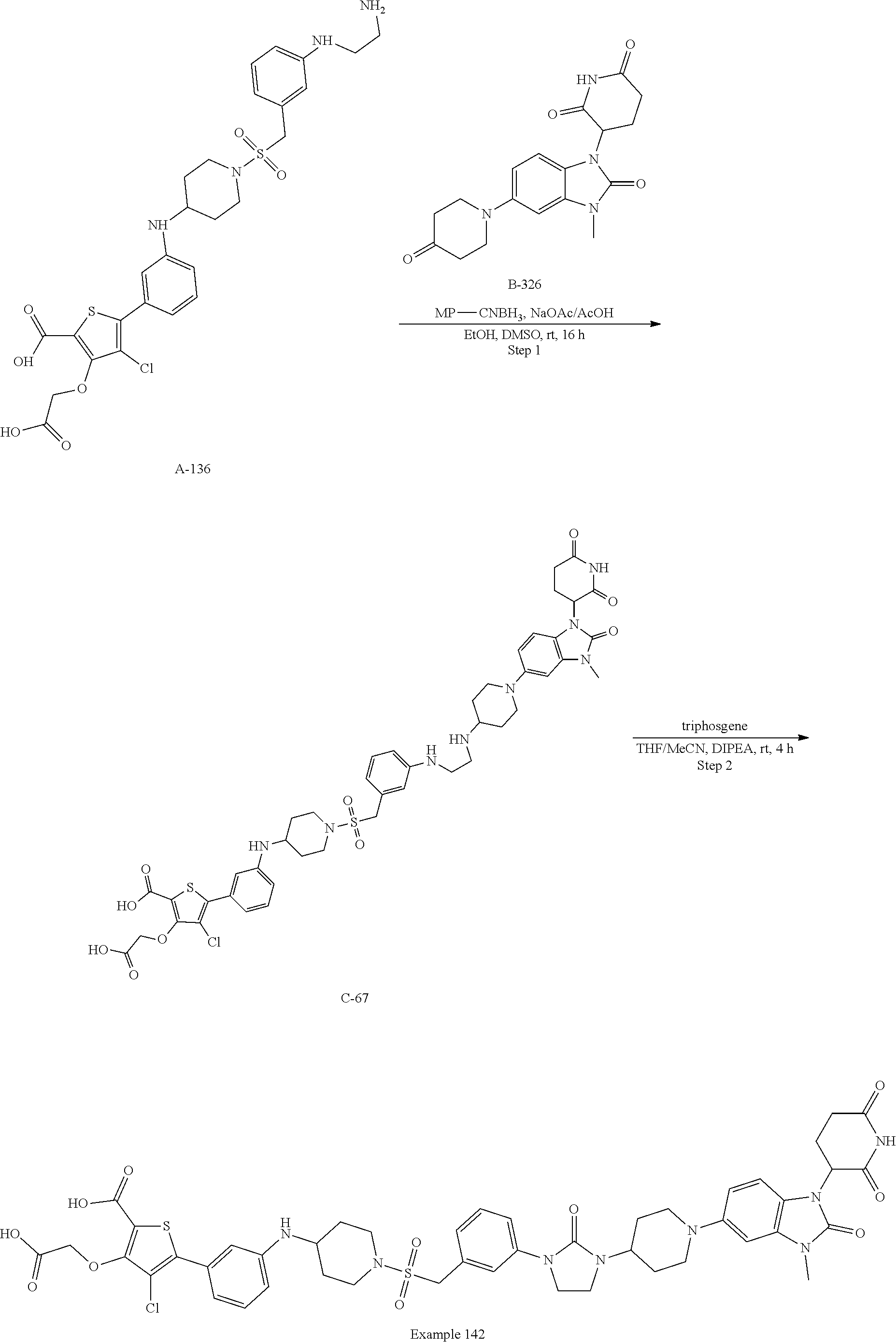

### Step 1: 3-(carboxymethoxy)-4-chloro-5-[3-[[1-[[3-[2-[1-[1-(2,6-dioxo-3-piperidyl)-3-methyl-2-oxo-benzimidazol-S-yl]-4-piperidyl]amino]ethylamino]phenyl]methylsulfonyl]-4-piperidyl]amino]phenyl]thiophene-2-carboxylic acid (C-67)

Into a 25 mL single neck round bottom flask containing a well-stirred solution of 5-[3-[[1-[[3-(2-aminoethylamino)phenyl]methylsulfonyl]-4-piperidyl]amino]phenyl]-3-(carboxymethoxy)-4-chloro-thiophene-2-carboxylic acid (A-136, 160 mg, 217.05 μmol, TFA salt) in ethanol (3 mL) were added 3-[3-methyl-2-oxo-5-(4-oxo-1-piperidyl)benzimidazol-1-yl]piperidine-2,6-dione (B-326, 77.35 mg, 164.44 μmol, TFA salt), anhydrous NaOAc (44.51 mg, 542.62 μmol) and acetic acid (65.17 mg, 1.09 mmol, 62.07 μL). After 1 h, MP-cyanoborohydride (197.95 mg, 394 μmol) was added and the reaction mixture was stirred for 16 h at ambient temperature. The reaction mixture was filtered through Celite, washed with ethanol (10 mL), filtered and evaporated under reduced pressure. The residue was purified by reverse-phase preparative-HPLC [Purification method: Column: XSelect C18 (150×19 mm), 5 μm; Solvent A: 0.1% TFA in water; Solvent B: Acetonitrile) to afford 3-(carboxymethoxy)-4-chloro-5-[3-[[1-[[3-[2-[[1-[1-(2,6-dioxo-3-piperidyl)-3-methyl-2-oxo-benzimidazol-5-yl]-4-piperidyl]amino]ethylamino]phenyl]methylsulfonyl]-4-piperidyl]amino]phenyl]thiophene-2-carboxylic acid (C-67, 90 mg, 73.63 μmol, 34% yield, TFA salt) as an off-white solid. LCMS (ES+): m/z 963.3 [M+H]⁺.

### Step 2: 3-(carboxymethoxy)-4-chloro-5-[3-[[1-[[3-[3-[[1-[1-(2,6-dioxo-3-piperidyl)-3-methyl-2-oxo-benzimidazol-5-yl]-4-piperidyl]-2-oxo-imidazolidin-1-yl]phenyl]methylsulfonyl]-4-piperidyl]amino]phenyl]thiophene-2-carboxylic acid (142)

Into a 10 mL single neck round bottom flask containing a well-stirred solution of 3-(carboxymethoxy)-4-chloro-5-[3-[[1-[[3-[2-[1-[1-(2,6-dioxo-3-piperidyl)-3-methyl-2-oxo-benzimidazol-5-yl]-4-piperidyl]amino]ethylamino]phenyl]methylsulfonyl]-4-piperidyl]amino]phenyl]thiophene-2-carboxylic acid (C-67, 90 mg, 83.52 μmol, TFA salt) in a mixture of anhydrous acetonitrile (1 mL) and THF (1 mL) were added triphosgene (49.57 mg, 167.05 μmol) and DIPEA (43.18 mg, 334.09 μmol, 58.19 μL). The reaction mixture was stirred at ambient temperature for 4 h. The reaction mixture was concentrated under reduced pressure and the residue was purified by reverse-phase column chromatography [Purification method: Column: X-BRIDGE C8 (19×150 mm), 5 μm; Mobile phase A: 0.1% TFA in water; Mobile phase B: Acetonitrile] to afford 3-(carboxymethoxy)-4-chloro-5-[3-[[1-[[3-[3-[[1-[1-(2,6-dioxo-3-piperidyl)-3-methyl-2-oxo-benzimidazol-5-yl]-4-piperidyl]-2-oxo-imidazolidin-1-yl]phenyl]methylsulfonyl]-4-piperidyl]amino]phenyl]thiophene-2-carboxylic acid (142, 7 mg, 6.66 μmol, 8% yield, TFA salt) as an off-white solid. LCMS (ES+): m/z 989.2 [M+H]⁺. ¹H NMR (400 MHz, DMSO-de) δ 11.07 (s, 1H), 7.68 (s, 1H), 7.57-7.48 (m, 1H), 7.33 (t, J=7.9 Hz, 1H), 7.20 (t, J=7.9 Hz, 1H), 7.06 (d, J=7.7 Hz, 1H), 6.94 (d, J=8.5 Hz, 1H), 6.89-6.84 (m, 2H), 6.81 (d, J=7.6 Hz, 1H), 6.72-6.64 (m, 2H), 5.87 (d, J=7.9 Hz, 1H), 5.29 (dd, J=12.9, 5.4 Hz, 1H), 4.85 (s, 2H), 4.39 (s, 2H), 3.85-3.76 (m, 3H), 3.72-3.64 (m, 2H), 3.02-2.84 (m, 4H), 2.77-2.67 (m, 3H), 2.03-1.68 (m, 9H), 1.41-1.29 (m, 2H).

### Example 143

### 3-(carboxymethoxy)-4-chloro-5-[3-[[(4S)-1-[[3-[[4-[3-(2,6-dioxo-3-piperidyl)-1-methyl-indazol-6-yl]piperidine-1-carbonyl]amino]phenyl]methylsulfonyl]-2,2-dimethyl-4-piperidyl]amino]phenyl]thiophene-2-carboxylic acid (143)

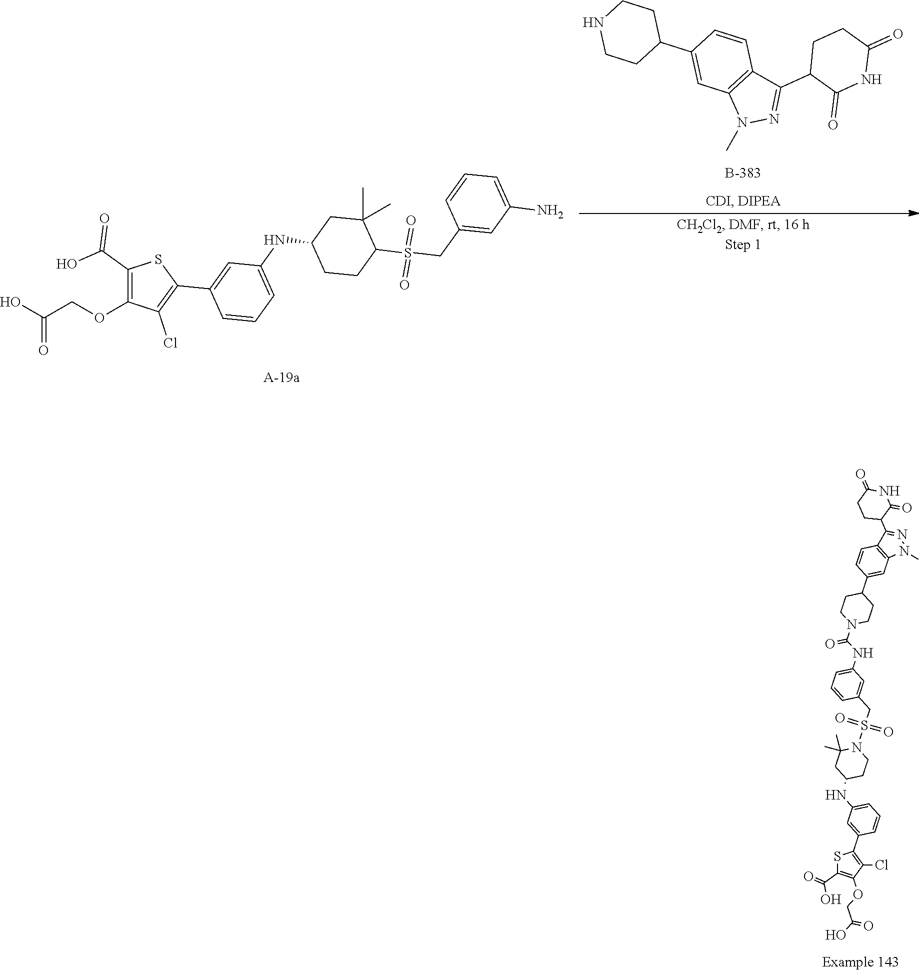 [SMILES: Cn1nc(C2CCC(=O)NC2=O)c2ccc(C3CCNCC3)cc21 ; CC1(C)C[C@@H](Nc2cccc(-c3sc(C(=O)O)c(OCC(=O)O)c3Cl)c2)CCC1S(=O)(=O)Cc1cccc(N)c1 ; Cn1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: 3-(carboxymethoxy)-4-chloro-5-[3-[[(4S)-1-[[3-[[4-[3-(2,6-dioxo-3-piperidyl)-1-methyl-indazol-6-yl]piperidine-1-carbonyl]amino]phenyl]methylsulfonyl]-2,2-dimethyl-4-piperidyl]amino]phenyl]thiophene-2-carboxylic acid (143)

3-(carboxymethoxy)-4-chloro-5-[3-[[(4S)-1-[[3-[[4-[3-(2,6-dioxo-3-piperidyl)-1-methyl-indazol-6-yl]piperidine-1-carbonyl]amino]phenyl]methylsulfonyl]-2,2-dimethyl-4-piperidyl]amino]phenyl]thiophene-2-carboxylic acid (143, 45 mg, 41.67 μmol, 19% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[1-methyl-6-(4-piperidyl) indazol-3-yl]piperidine-2,6-dione (B-383) in a similar fashion to Example 23, except using excess DIPEA as base and DCM:DMF as the solvent. The residue was purified by reverse-phase column chromatography [Purification method: Column: XSelect (150×19 mm) 5 μm; Mobile phase A: 0.1% TFA in MQ-water; Mobile phase B: CH₃CN]. LCMS (ES+): m/z 961.2 [M+H]⁺. ¹H NMR (400 MHz, DMSO-d₆) δ 10.88 (s, 1H), 8.64 (s, 1H), 7.63-7.56 (m, 2H), 7.49-7.44 (m, 2H), 7.27-7.16 (m, 2H), 7.05 (d, J=8.5 Hz, 1H), 6.99 (d, J=7.6 Hz, 1H), 6.90-6.87 (m, 1H), 6.84-6.79 (m, 1H), 6.73-6.68 (m, 1H), 4.90 (s, 2H), 4.40-4.21 (m, 5H), 3.96 (s, 3H), 3.12 (t, J=12.6 Hz, 1H), 2.97-2.83 (m, 3H), 2.71-2.56 (m, 2H), 2.39-2.29 (m, 1H), 2.20-2.11 (m, 1H), 1.90-1.62 (m, 6H), 1.48 (s, 3H), 1.46-1.36 (m, 4H), 1.18-1.04 (m, 1H).

### Example 144

### 3-(carboxymethoxy)-4-chloro-5-[3-[[(4S)-1-[3-[4-[[1-(2,6-dioxo-3-piperidyl)-3-methyl-2-oxo-benzimidazol-5-yl]amino]benzoyl]amino]phenyl]methylsulfonyl]-2,2-dimethyl-4-piperidyl]amino]phenyl]thiophene-2-carboxylic acid (144)

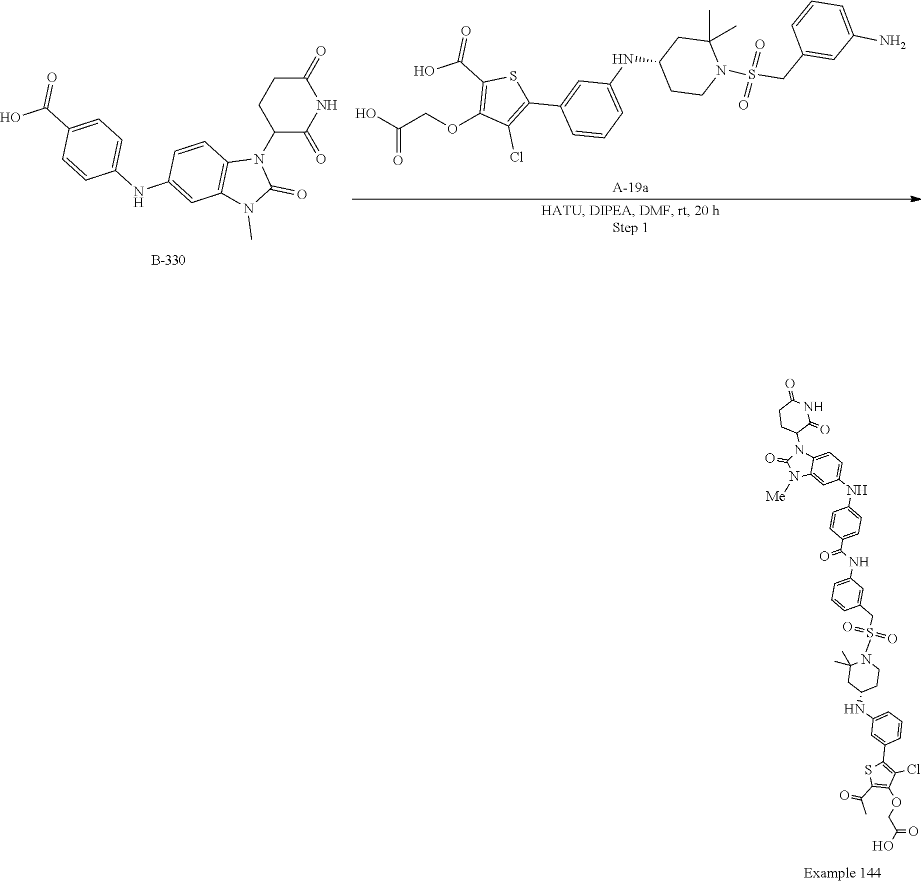 [SMILES: Cn1c(=O)n(C2CCC(=O)NC2=O)c2ccc(Nc3ccc(C(=O)O)cc3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; CC(=O)c1sc(-c2cccc(N[C@H]3CCN(S(=O)(=O)Cc4cccc(NC(=O)c5ccc(Nc6ccc7c(c6)n(C)c(=O)n7C6CCC(=O)NC6=O)cc5)c4)C(C)(C)C3)c2)c(Cl)c1OCC(=O)O]

### Step 1: 3-(carboxymethoxy)-4-chloro-5-[3-[[(4S)-1-[[3-[[4-[[1-(2,6-dioxo-3-piperidyl)-3-methyl-2-oxo-benzimidazol-5-yl]amino]benzoyl]amino]phenyl]methylsulfonyl]-2,2-dimethyl-4-piperidyl]amino]phenyl]thiophene-2-carboxylic acid (144)

3-(carboxymethoxy)-4-chloro-5-[3-[[(4S)-1-[[3-[[4-[1-(2,6-dioxo-3-piperidyl)-3-methyl-2-oxo-benzimidazol-5-yl]amino]benzoyl]amino]phenyl]methylsulfonyl]-2,2-dimethyl-4-piperidyl]amino]phenyl]thiophene-2-carboxylic acid (144, 23 mg, 19.71 μmol, 12% yield, TFA salt) was synthesized from 4-[[1-(2,6-dioxo-3-piperidyl)-3-methyl-2-oxo-benzimidazol-5-yl]amino]benzoic acid (B-330) and 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) in a similar fashion to Example 1 except using DMF as the solvent. The residue was purified by reverse phase prep HPLC [Purification method: Column: Zorbax C18 (250×21.2 mm) 7 μm; Mobile phase A: 0.1% TFA in water and Mobile phase B: Acetonitrile]. LCMS (ES+): m/z 985.0 [M+H]⁺. ¹H NMR (400 MHz, DMSO-d₆) δ 11.11 (s, 1H), 10.01 (s, 1H), 7.90-7.84 (m, 3H), 7.77-7.73 (m, 1H), 7.33 (t, J=7.9 Hz, 1H), 7.19 (t, J=7.9 Hz, 1H), 7.12-7.05 (m, 2H), 7.05-7.00 (m, 3H), 6.89-6.85 (m, 2H), 6.83-6.79 (m, 1H), 6.70-6.66 (m, 1H), 5.35 (dd, J=12.7, 5.4 Hz, 1H), 4.91 (s, 2H), 4.41 (d, J=13.8 Hz, 1H), 4.30 (d, J=13.8 Hz, 1H), 3.32 (s, 3H), 3.17-3.07 (m, 2H), 2.95-2.85 (m, 2H), 2.77-2.59 (m, 2H), 2.09-2.00 (m, 1H), 1.92-1.85 (m, 1H), 1.82-1.76 (m, 1H), 1.49 (s, 3H), 1.46-1.37 (m, 4H).

### Example 145

### 3-(carboxymethoxy)-4-chloro-5-(3-(((4S)-1-((3-(3-(4-(1-(2,6-dioxopiperidin-3-yl)-3-methyl-2-oxo-2,3-dihydro-1H-benzo[d]imidazol-5-yl)phenyl)ureido)benzyl)sulfonyl)-2,2-dimethylpiperidin-4-yl)amino)phenyl)thiophene-2-carboxylic acid (145)

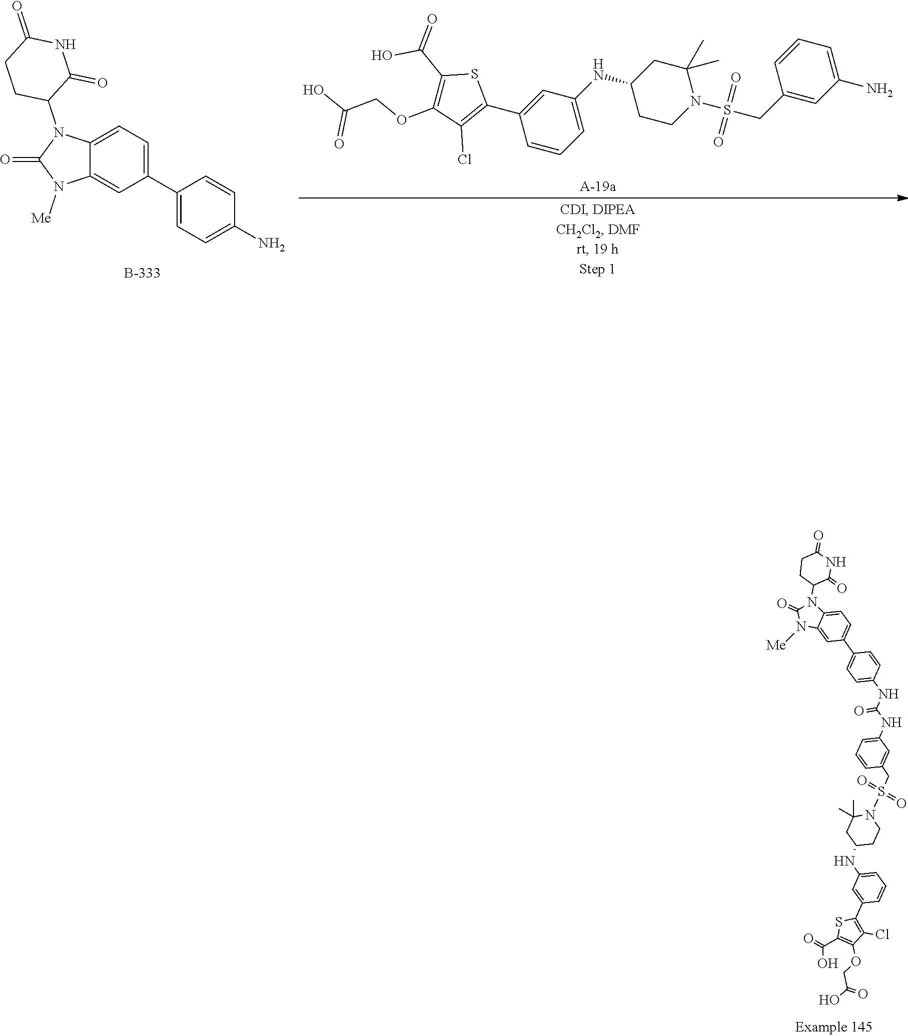 [SMILES: Cn1c(=O)n(C2CCC(=O)NC2=O)c2ccc(-c3ccc(N)cc3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(-c3ccc(NC(=O)Nc4cccc(CS(=O)(=O)N5CC[C@H](Nc6cccc(-c7sc(C(=O)O)c(OCC(=O)O)c7Cl)c6)CC5(C)C)c4)cc3)cc21]

### Step 1: 3-(carboxymethoxy)-4-chloro-5-(3-(((4S)-1-((3-(3-(4-(1-(2,6-dioxopiperidin-3-yl)-3-methyl-2-oxo-2,3-dihydro-1H-benzo[d]imidazol-S-yl)phenyl)ureido)benzyl)sulfonyl)-2,2-dimethylpiperidin-4-yl)amino)phenyl)thiophene-2-carboxylic acid (145)

3-(carboxymethoxy)-4-chloro-5-(3-(((4S)-1-((3-(3-(4-(1-(2,6-dioxopiperidin-3-yl)-3-methyl-2-oxo-2,3-dihydro-1H-benzo[d]imidazol-5-yl)phenyl)ureido)benzyl)sulfonyl)-2,2-dimethylpiperidin-4-yl)amino)phenyl)thiophene-2-carboxylic acid (145, 11 mg, 5% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-(5-(4-aminophenyl)-3-methyl-2-oxo-2,3-dihydro-1H-benzo[d]imidazol-1-yl)piperidine-2,6-dione (B-333) in a similar fashion to Example 23, except using excess DIPEA as base and DCM:DMF as the solvent. The residue was purified by reverse phase preparative HPLC [Purification method: Column: Zorbax C18 (250×21.2 mm) 7 μm, Mobile phase A: 0.1% TFA in water; Mobile phase B: Acetonitrile). LCMS (ES+): m/z 984.2 [M+H]⁺. ¹H NMR (400 MHz, DMSO-d₆) δ 11.12 (s, 1H), 8.78 (d, J=20.8 Hz, 2H), 7.65-7.60 (m, 2H), 7.58-7.53 (m, 3H), 7.47-7.43 (m, 2H), 7.33-7.27 (m, 2H), 7.19-7.13 (m, 2H), 7.02 (d, J=7.6 Hz, 1H), 6.87-6.84 (m, 1H), 6.81-6.77 (m, 1H), 6.65-6.60 (m, 1H), 5.39 (dd, J=12.7, 5.4 Hz, 1H), 4.89 (s, 2H), 4.42 (d, J=13.8 Hz, 1H), 4.29 (d, J=13.8 Hz, 1H), 2.96-2.85 (m, 2H), 2.79-2.70 (m, 2H), 2.10-2.01 (m, 1H), 1.93-1.84 (m, 1H), 1.81-1.76 (m, 1H), 1.46 (s, 3H), 1.43-1.36 (m, 4H), 1.20-1.07 (m, 1H).

### Example 146

### 3-(carboxymethoxy)-4-chloro-5-[3-[[1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (146)

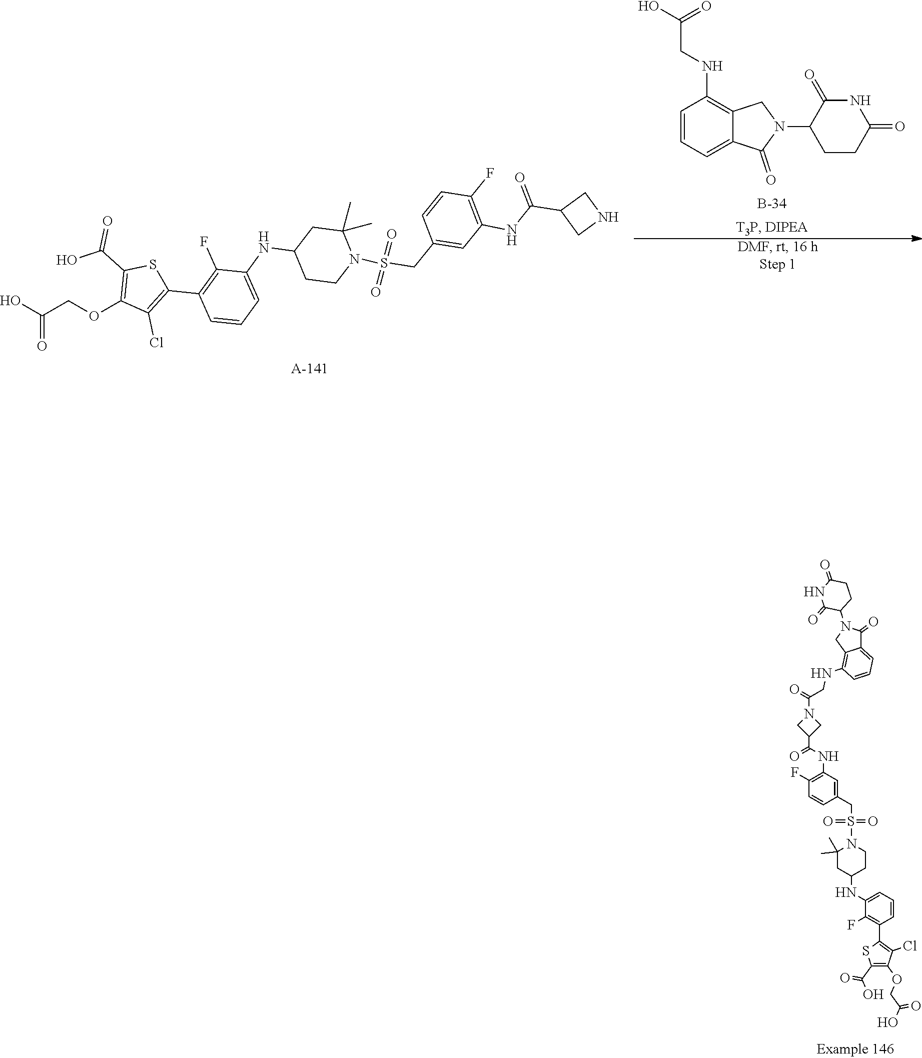

### Step 1: 3-(carboxymethoxy)-4-chloro-5-[3-[[1-[[3-[1-[2-[[2-(2,6-dioxo-3-piperidyl)-1-oxo-isoindolin-4-yl]amino]acetyl]azetidine-3-carbonyl]amino]-4-fluoro-phenyl]-methylsulfonyl]-2,2-dimethyl-4-piperidyl]amino]-2-fluoro-phenyl]thiophene-2-carboxylic acid (146)

3-(carboxymethoxy)-4-chloro-5-[3-[[1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (146, 59 mg, 48.58 μmol, 50% yield, TFA salt) was synthesized from 2-[[2-(2,6-dioxo-3-piperidyl)-1-oxo-isoindolin-4-yl]amino]acetic acid (B-34) and 5-[3-[[1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-141) in a similar fashion to Compound C-6, except using DIPEA as the base. The residue was purified by reverse-phase preparative-HPLC [Purification method: Column: XSelect C18 (150×19 mm) 5 μm; Mobile phase A: 0.1% TFA in water and Mobile phase B: CH₃CN]. LCMS (ES+): m/z 1026.2 [M+H]⁺. ¹H NMR (400 MHz, DMSO-d₆) δ 11.01 (s, 1H), 9.98 (s, 1H), 8.00 (d, J=7.5 Hz, 1H), 7.32-7.25 (m, 2H), 7.24-7.19 (m, 1H), 7.12-7.05 (m, 1H), 7.00-6.92 (m, 2H), 6.68-6.61 (m, 2H), 5.11 (dd, J=13.3, 5.1 Hz, 1H), 4.94-4.89 (m, 2H), 4.48-4.32 (m, 4H), 4.28 (d, J=19.0 Hz, 1H), 4.16 (d, J=17.1 Hz, 1H), 4.10-3.97 (m, 3H), 3.47-3.41 (m, 2H), 3.13 (t, J=12.6 Hz, 1H), 2.92 (ddd, J=18.0, 13.5, 5.4 Hz, 1H), 2.65-2.57 (m, 2H), 2.07-1.98 (m, 1H), 1.91-1.83 (m, 1H), 1.78-1.70 (m, 1H), 1.65-1.53 (m, 1H), 1.47 (s, 3H), 1.40 (s, 3H), 1.28-1.15 (m, 1H).

### Example 147

### 3-(carboxymethoxy)-4-chloro-5-[3-[[(4S)-1-[[3-[[4-[1-(2,6-dioxo-3-piperidyl)-3-methyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylic acid (147)

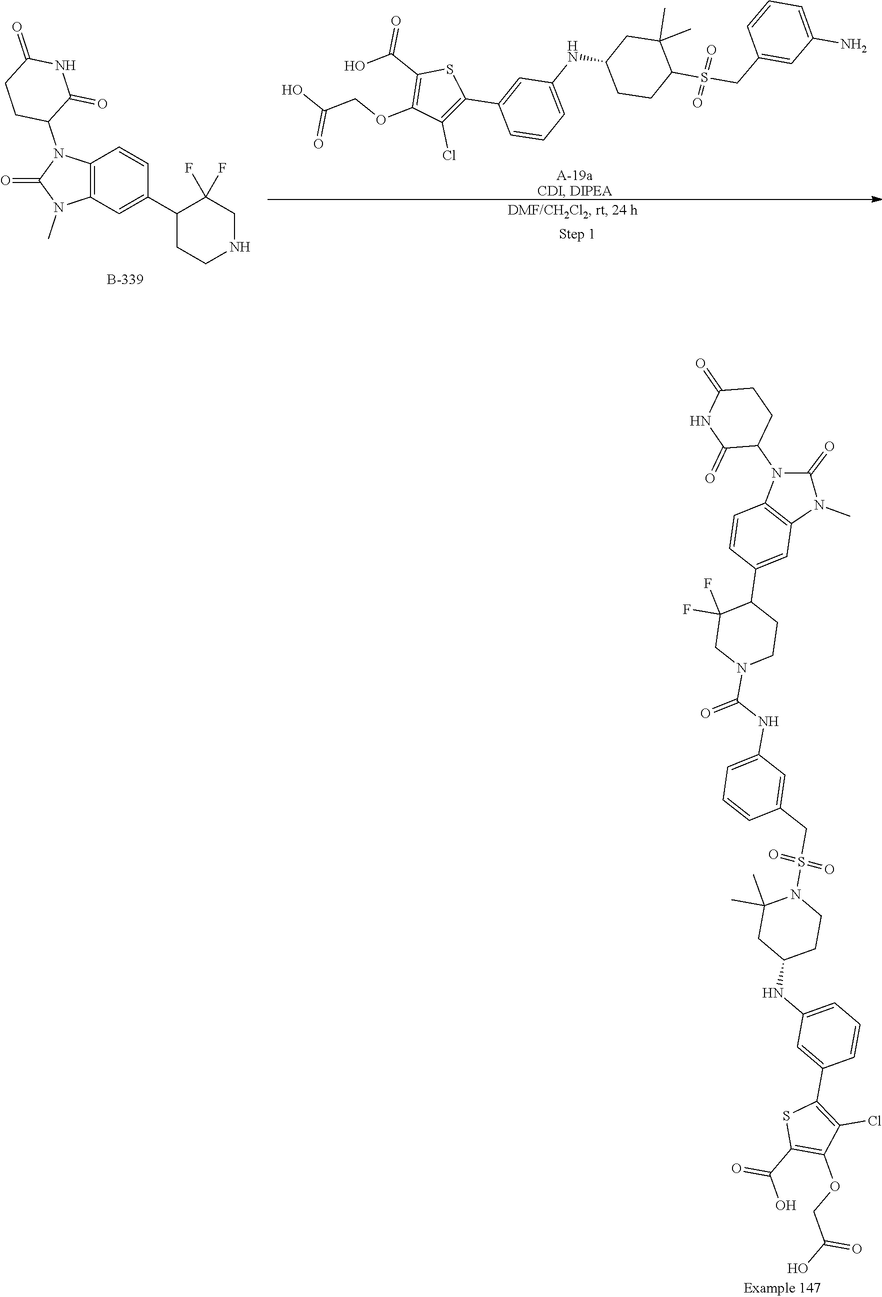 [SMILES: Cn1c(=O)n(C2CCC(=O)NC2=O)c2ccc(C3CCNCC3(F)F)cc21 ; CC1(C)C[C@@H](Nc2cccc(-c3sc(C(=O)O)c(OCC(=O)O)c3Cl)c2)CCC1S(=O)(=O)Cc1cccc(N)c1 ; Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21]

### Step 1: 3-(carboxymethoxy)-4-chloro-5-[3-[[(4S)-1-[[3-[[4-[1-(2,6-dioxo-3-piperidyl)-3-methyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylic acid (147)

3-(carboxymethoxy)-4-chloro-5-[3-[[(4S)-1-[[3-[[4-[1-(2,6-dioxo-3-piperidyl)-3-methyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylic acid (147, 62 mg, 51.94 μmol, 9% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[5-(3,3-difluoro-4-piperidyl)-3-methyl-2-oxo-benzimidazol-1-yl]piperidine-2,6-dione (B-339) in a similar fashion to Example 23, except using excess DIPEA as base and DCM:DMF as the solvent. The residue was purified by reverse phase preparative HPLC (Column: XSelect-C18 (19×150 mm) 5 μm; Mobile phase A: 0.1% TFA in water; Mobile phase B: MeCN). LCMS (ES+): m/z 1012 [M+H]⁺. ¹H NMR (400 MHz, DMSO-d₆) δ 11.11 (s, 1H), 8.80 (s, 1H), 7.59-7.56 (m, 1H), 7.52-7.47 (m, 1H), 7.27 (t, J=7.9 Hz, 1H), 7.20 (t, J=7.9 Hz, 1H), 7.15 (s, 1H), 7.07 (d, J=8.1 Hz, 1H), 7.04-6.97 (m, 2H), 6.90-6.86 (m, 1H), 6.83-6.79 (m, 1H), 6.73-6.67 (m, 1H), 5.37 (dd, J=12.8, 5.4 Hz, 1H), 4.91 (s, 2H), 4.65-4.54 (m, 1H), 4.42-4.33 (m, 2H), 4.27 (d, J=13.8 Hz, 1H), 3.59-3.40 (m, 3H), 3.38-3.24 (m, 4H), 3.19-3.00 (m, 2H), 2.95-2.84 (m, 1H), 2.77-2.57 (m, 2H), 2.25-2.12 (m, 1H), 2.05-1.97 (m, 1H), 1.92-1.83 (m, 2H), 1.79 (d, J=12.8 Hz, 1H), 1.49 (d, J=5.4 Hz, 3H), 1.42-1.36 (m, 4H), 1.17-1.05 (m, 1H).

### Example 148

### 3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (148)

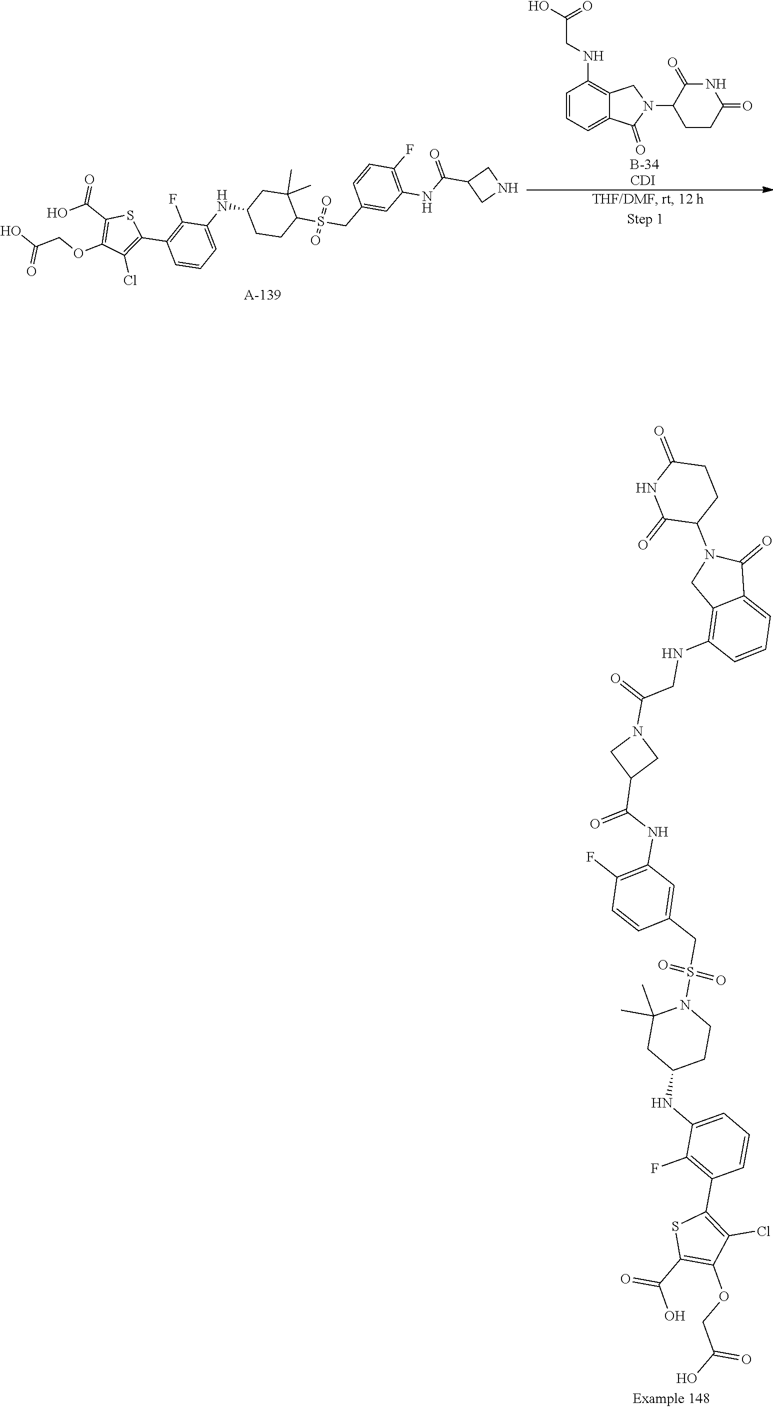

### Step 1: 3-(carboxymethoxy)-4-chloro-5-[3-[[(4S)-1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (148)

3-(carboxymethoxy)-4-chloro-5-[3-[[(4S)-1-[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (148, 110.06 mg, 95.15 μmol, 58% yield, TFA salt) was synthesized from 2-[[2-(2,6-dioxo-3-piperidyl)-1-oxo-isoindolin-4-yl]amino]acetic acid (B-34) and 5-[3-[[(4S)-1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-139) in a similar fashion to Example 23, except using DMF/THF as the solvent. The residue was purified by reverse-phase preparative-HPLC [Purification method: Column: SunFire C18 (150×19 mm) 5 μm; Mobile phase A: 0.1% TFA in water and Mobile phase B: CH₃CN]. LCMS (ES+): m/z 1026.2 [M+H]⁺. ¹H NMR (400 MHz, DMSO-d₆) δ 11.00 (s, 1H), 9.97 (s, 1H), 8.00 (d, J=7.4 Hz, 1H), 7.32-7.24 (m, 2H), 7.24-7.18 (m, 1H), 7.08 (t, J=7.9 Hz, 1H), 7.00-6.91 (m, 2H), 6.68-6.61 (m, 2H), 5.11 (dd, J=13.2, 5.1 Hz, 1H), 4.93-4.89 (m, 2H), 4.48-4.32 (m, 4H), 4.28 (d, J=18.8 Hz, 1H), 4.16 (d, J=17.0 Hz, 1H), 4.10-3.95 (m, 2H), 3.86-3.81 (m, 2H), 3.75-3.70 (m, 2H), 3.18-3.08 (m, 1H), 2.98-2.86 (m, 1H), 2.63-2.55 (m, 2H), 2.05-1.98 (m, 1H), 1.91-1.82 (m, 1H), 1.78-1.71 (m, 1H), 1.58 (t, J=12.3 Hz, 1H), 1.47 (s, 3H), 1.40 (s, 3H), 1.30-1.17 (m, 1H).

### Example 148a and Example 148b

### 3-(carboxymethoxy)-4-chloro-5-[2-fluoro-3-[(4S)-1-[[4-fluoro-3-[[1-[2-[[1-oxo-2-[(3S)-2,6-dioxo-3-piperidyl]isoindolin-4-yl]amino]acetyl]azetidine-3-carbonyl]amino]phenyl]methylsulfonyl]-2,2-dimethyl-4-piperidyl]amino]phenyl]thiophene-2-carboxylic acid (148a) and 3-(carboxymethoxy)-4-chloro-5-[2-fluoro-3-[(4S)-1-[[4-fluoro-3-[1-[2-[1-oxo-2-[(3R)-2,6-dioxo-3-piperidyl]isoindolin-4-yl]amino]acetyl]azetidine-3-carbonyl]amino]phenyl]methylsulfonyl]-2,2-dimethyl-4-piperidyl]amino]phenyl]thiophene-2-carboxylic acid (148b)

Configurations are arbitrarily assigned.

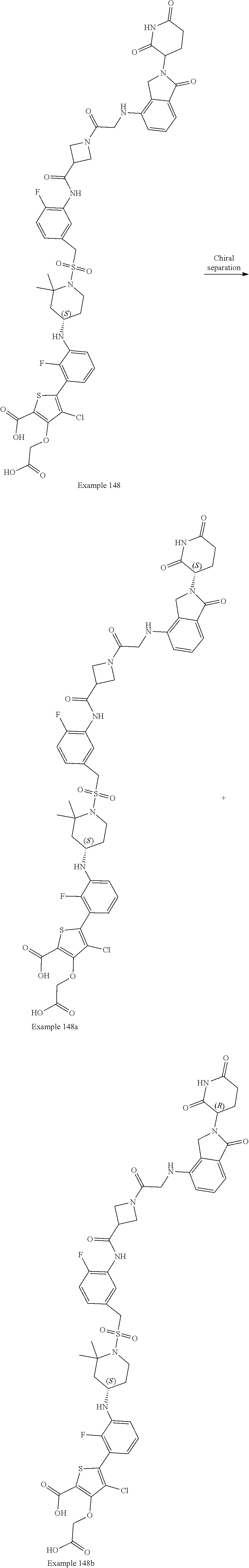

3-(carboxymethoxy)-4-chloro-5-[2-fluoro-3-[[(4S)-1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]phenyl]thiophene-2-carboxylic acid (148, 50 mg, 43.84 μmol, TFA salt) was subjected to chiral SFC purification to separate the diastereomers. Method details: Column Name: YMC Cellulose-SC, Flow Rate: 5 mL/min, Co-Solvent: 50%, Co-Solvent Name: 0.2% TFA in IPA:ACN (1:1), Injected Volume: 15 μL, Temperature: 35° C., Outlet Pressure: 100 bar.

### Chiral SFC:

### Method of Analysis

Instrument: PIC 175
Column: YMC Cellulose SC (250×30) mm, 5 μm.
Mobile Phase: CO2: 0.2% Trifluoroacetic acid in Isopropyl alcohol:Acetonitrile (1:1):(50:50)
Total Flow: 90 g/min
Back pressure: 120 bar
Wave length: 210 nm
Cycle time: 25 min

50 mg of C869551 was dissolved in 2 mL of ACN and injected 900 μL/injection.

After SFC purification, the first eluted fraction at rt 10.6 min was concentrated under reduced pressure at 40° C. and purified by reverse-phase preparative column chromatography [Silicycle C18 column, mobile phase: 0.1% TFA in water and MeCN] to afford 3-(carboxymethoxy)-4-chloro-5-[2-fluoro-3-[[(4S)-1-[[4-fluoro-3-[[1-[2-[[1-oxo-2-[(3S)-2,6-dioxo-3-piperidyl]isoindolin-4-yl]amino]acetyl]azetidine-3-carbonyl]amino]phenyl]methylsulfonyl]-2,2-dimethyl-4-piperidyl]amino]phenyl]thiophene-2-carboxylic acid (148a, 18 mg, 14.89 μmol, 34% yield, TFA salt) as an off-white solid.

### Fraction 1: 148a:

¹H-NMR (400 MHz, DMSO-d6): δ 11.00 (s, 1H), 10.00 (s, 1H), 7.99 (d, J=6.40 Hz, 1H), 7.32-7.20 (m, 3H), 7.09 (t, J=7.60 Hz, 1H), 6.99-6.92 (m, 2H), 6.67-6.62 (m, 2H), 5.78 (bs, 1H), 5.32 (bs, 1H), 5.15-5.06 (m, 1H), 4.88 (s, 2H), 4.51-4.15 (m, 7H), 4.06-3.98 (m, 2H), 3.84 (s, 2H), 3.74-3.72 (m, 2H), 2.94-2.86 (m, 2H), 2.04-1.99 (m, 1H), 1.89-1.82 (m, 1H), 1.78-1.72 (m, 2H), 1.64-1.55 (m, 2H), 1.40 (s, 3H), 1.24 (s, 3H), 1.98-1.09 (m, 1H).

LCMS (ES+): m/z 1026.1 [M+H]⁺

Similarly, the second eluted fraction at rt 16.0 min was concentrated under reduced pressure at 40° C. and purified by reverse-phase preparative column chromatography [Silicycle C18 column, mobile phase: 0.1% TFA in water and MeCN] to afford 3-(carboxymethoxy)-4-chloro-5-[2-fluoro-3-[[(4S)-1-[4-fluoro-3-[[1-[2-[[1-oxo-2-[(3R)-2,6-dioxo-3-piperidyl]isoindolin-4-yl]amino]acetyl]azetidine-3-carbonyl]amino]phenyl]methylsulfonyl]-2,2-dimethyl-4-piperidyl]amino]phenyl]thiophene-2-carboxylic acid (148b, 16 mg, 12.79 μmol, 29% yield, TFA salt) as an off-white solid.

### Fraction 2: 148b:

¹H-NMR (400 MHz, DMSO-d6): δ 11.00 (s, 1H), 10.00 (s, 1H), 7.99 (d, J=6.00 Hz, 1H), 7.31-7.22 (m, 3H), 7.08 (t, J=8.00 Hz, 1H), 6.99-6.91 (m, 2H), 6.68-6.62 (m, 2H), 5.69 (s, 1H), 5.32 (s, 1H), 5.15-5.06 (m, 1H), 4.81 (s, 2H), 4.45-4.27 (m, 7H), 4.09-3.98 (m, 2H), 3.83 (s, 2H), 3.74-3.72 (m, 2H), 2.95-2.85 (m, 2H), 2.06-1.99 (m, 1H), 1.89-1.82 (m, 1H), 1.78-1.73 (m, 2H), 1.64-1.55 (m, 2H), 1.47 (s, 3H), 1.40 (s, 3H), 1.98-1.15 (m, 2H).

LCMS (ES+): m/z 1026.1 [M+H]⁺

### Example 149

### 3-(carboxymethoxy)-4-chloro-5-[3-[(4R)-1-[[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (149)

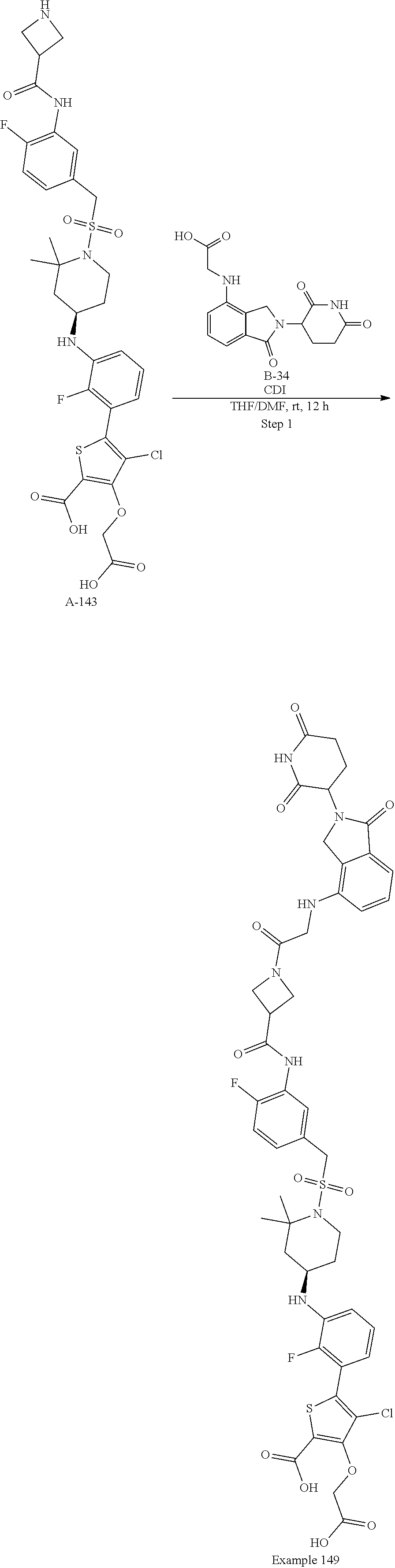

### Step 1: 3-(carboxymethoxy)-4-chloro-5-[3-[[(4R)-1-1 [3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (149)

3-(carboxymethoxy)-4-chloro-5-[3-[(4R)-1-[3-[[1-[2-[[2-(2,6-dioxo-3-piperidyl)-1-oxo-isoindolin-4-yl]amino]acetyl]azetidine-3-carbonyl]amino]-4-fluoro-phenyl]methylsulfonyl]-2,2-dimethyl-4-piperidyl]amino]-2-fluoro-phenyl]thiophene-2-carboxylic acid (149, 96.19 mg, 42% yield, Formic acid salt) was synthesized from (2-(2,6-dioxopiperidin-3-yl)-1-oxoisoindolin-4-yl)glycine (B-34) and 5-[3-[[(4R)-1-[[3-(azetidine-3-carbonylamino)-4-fluoro-phenyl]methylsulfonyl]-2,2-dimethyl-4-piperidyl]amino]-2-fluoro-phenyl]-3-(carboxymethoxy)-4-chloro-thiophene-2-carboxylic acid (A-143) in a similar fashion to Example 23, except using DMF/THE as the solvent. The residue was purified using reverse phase prep HPLC [Purification method: SiliCycle C18 (150×19 mm), 5 μm, Mobile phase A: 0.1% Formic acid in water; Mobile phase B: MeCN]. LCMS (ES+): m/z 1026.2 [M+H]⁺. ¹H NMR (400 MHz, DMSO-d₆) δ 11.01 (s, 1H), 9.98 (s, 1H), 8.02-7.97 (m, 1H), 7.35-7.24 (m, 2H), 7.24-7.18 (m, 1H), 7.08 (t, J=7.9 Hz, 1H), 7.00-6.90 (m, 2H), 6.69-6.61 (m, 2H), 5.78 (s, 1H), 5.38-5.29 (m, 1H), 5.11 (dd, J=13.2, 5.1 Hz, 1H), 4.89 (d, J=3.7 Hz, 2H), 4.48-4.31 (m, 4H), 4.28 (d, J=18.3 Hz, 1H), 4.16 (d, J=17.1 Hz, 1H), 4.09-3.96 (m, 2H), 3.83 (s, 2H), 3.75-3.69 (m, 1H), 3.66-3.57 (m, 1H), 3.13 (t, J=12.7 Hz, 1H), 2.92 (ddd, J=18.0, 13.6, 5.5 Hz, 1H), 2.64-2.55 (m, 2H), 2.05-1.99 (m, 1H), 1.89-1.81 (m, 1H), 1.78-1.72 (m, 1H), 1.58 (t, J=12.4 Hz, 1H), 1.47 (s, 3H), 1.40 (s, 3H), 1.28-1.16 (m, 1H).

### Example 150

### 3-(carboxymethoxy)-4-chloro-5-[3-[[(4S)-1-[3-[4-[1′-(2,6-dioxo-3-piperidyl)-2′-oxo-spiro[cyclopropane-1,3′-indoline]-5′-yl]piperidine-1-carbonyl]amino]phenyl]methylsulfonyl]-2,2-dimethyl-4-piperidyl]amino]phenyl]thiophene-2-carboxylic acid (150)

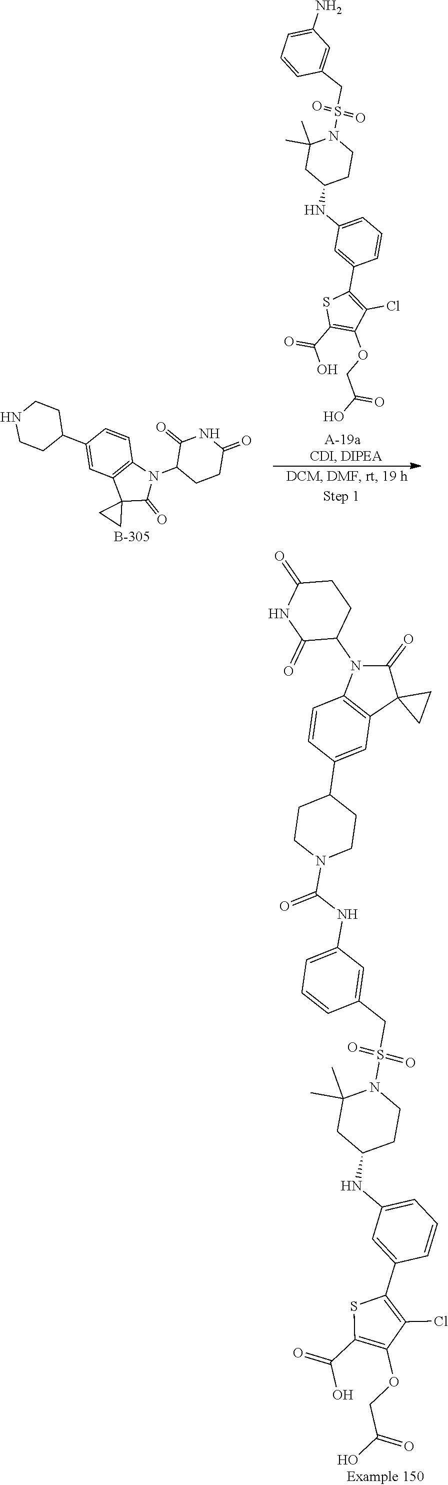 [SMILES: O=C1CCC(N2C(=O)C3(CC3)c3cc(C4CCNCC4)ccc32)C(=O)N1 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)C3(CC3)C(=O)N4C3CCC(=O)NC3=O)CC2)c1]

### Step 1: 3-(carboxymethoxy)-4-chloro-5-[3-[[(4S)-1-[[3-[[4-[1′-(2,6-dioxo-3-piperidyl)-2′-oxo-spiro[cyclopropane-1,3′-indoline]-S′-yl]piperidine-1-carbonyl]amino]phenyl]methylsulfonyl]-2,2-dimethyl-4-piperidyl]amino]phenyl]thiophene-2-carboxylic acid (150)

3-(carboxymethoxy)-4-chloro-5-[3-[[(4S)-1-[[3-[[4-[1′-(2,6-dioxo-3-piperidyl)-2′-oxo-spiro[cyclopropane-1,3′-indoline]-5′-yl]piperidine-1-carbonyl]amino]phenyl]methylsulfonyl]-2,2-dimethyl-4-piperidyl]amino]phenyl]thiophene-2-carboxylic acid (150, 3.65 mg, 3% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[2′-oxo-5′-(4-piperidyl)spiro[cyclopropane-1,3′-indoline]-1′-yl]piperidine-2,6-dione (B-305) in a similar fashion to Example 23, except using excess DIPEA as base and DMF/THF as the solvent. The residue was purified by reverse phase prep HPLC [Purification method: Column: X-bridge C8, Mobile phase A: 0.1% TFA in water; Mobile phase B: MeCN). LCMS (ES+): m/z 988.5 [M+H]⁺. ¹H NMR (400 MHz, DMSO-d₆): δ 11.07 (s, 1H), 8.60 (s, 1H), 7.56 (s, 1H), 7.45 (d, J=8.40 Hz, 1H), 7.26-7.18 (m, 2H), 7.08 (d, J=8.00 Hz, 1H), 7.00-6.80 (m, 5H), 6.69 (dd, J=1.60, 8.20 Hz, 1H), 5.35-5.25 (m, 1H), 4.91 (s, 2H), 4.37-4.23 (m, 4H), 3.46-3.12 (m, 3H), 2.89-2.83 (m, 4H), 2.67-2.59 (m, 3H), 1.97-1.94 (m, 1H), 1.87-1.74 (m, 4H), 1.66-1.64 (m, 2H), 1.61-1.43 (m, 7H), 1.39 (s, 3H), 1.15-1.05 (m, 1H).

### Example 151

### 3-(carboxymethoxy)-4-chloro-5-[3-[[(4S)-1-[3-[4-[1-(2,6-dioxo-3-piperidyl)-3-methyl-2-oxo-benzimidazol-4-yl]oxypiperidine-1-carbonyl]amino]phenyl]methylsulfonyl]-2,2-dimethyl-4-piperidyl]amino]phenyl]thiophene-2-carboxylic acid (151)

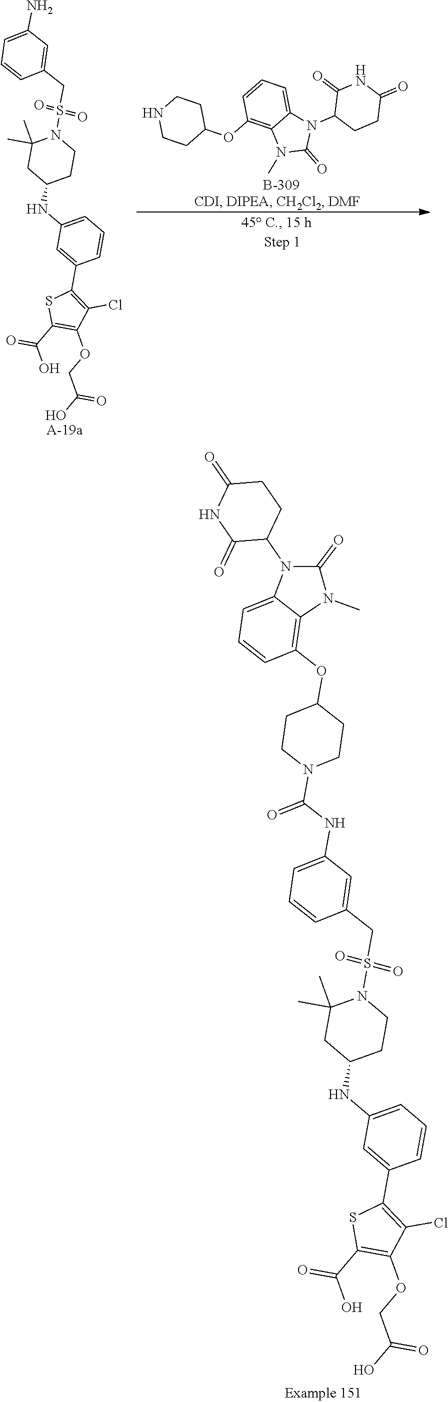 [SMILES: CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2cccc(OC3CCNCC3)c21 ; Cn1c(=O)n(C2CCC(=O)NC2=O)c2cccc(OC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)c21]

### Step 1: 3-(carboxymethoxy)-4-chloro-5-[3-[[(4S)-1-[[3-[[4-[1-(2,6-dioxo-3-piperidyl)-3-methyl-2-oxo-benzimidazol-4-yl]oxypiperidine-1-carbonyl]amino]phenyl]methylsulfonyl]-2,2-dimethyl-4-piperidyl]amino]phenyl]thiophene-2-carboxylic acid (151)

3-(carboxymethoxy)-4-chloro-5-[3-[[(4S)-1-[[3-[[4-[1-(2,6-dioxo-3-piperidyl)-3-methyl-2-oxo-benzimidazol-4-yl]oxypiperidine-1-carbonyl]amino]phenyl]methylsulfonyl]-2,2-dimethyl-4-piperidyl]amino]phenyl]thiophene-2-carboxylic acid (151, 17 mg, 16.26% yield, Formic acid salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-methyl-2-oxo-4-(4-piperidyloxy)benzimidazol-1-yl]piperidine-2,6-dione (B-309) in a similar fashion to Example 23, except using excess DIPEA as base and DMF/THE as the solvent. The residue was purified by reverse phase prep HPLC [Purification method: X-BRIDGE C8 (19×150 mm), 5 μm; Mobile phase A: 0.1% Formic acid in water, Mobile phase B: MeCN]. LCMS (ES+): m/z 992.3 [M+H]⁺. ¹H NMR (400 MHz, DMSO-d₆): δ 11.11 (s, 1H), 8.67 (s, 1H), 7.55 (s, 1H), 7.46 (d, J=8.00 Hz, 1H), 7.19-7.25 (m, 2H), 7.00-6.95 (m, 2H), 6.85-6.81 (m, 2H), 6.79-6.74 (m, 2H), 6.68 (d, J=8.40 Hz, 1H), 5.69 (d, J=8.00 Hz, 1H), 5.35 (dd, J=5.20, 12.40 Hz, 1H), 4.86 (s, 2H), 4.77-4.69 (m, 1H), 4.31 (dd, J=13.60, 44.4 Hz, 2H), 3.78-3.74 (m, 2H), 3.52-3.56 (m, 4H), 3.15-3.10 (m, 2H), 2.94-2.85 (m, 1H), 2.67-2.60 (m, 1H), 2.02-1.99 (m, 4H), 1.87-1.80 (m, 2H), 1.76-1.71 (m, 3H), 1.45-1.55 (m, 4H), 1.44 (m, 4H), 1.27-1.22 (m, 1H), 1.13-1.21 (m, 1H), 1.85-1.93 (m, 1H).

### Example 152

### 3-(carboxymethoxy)-4-chloro-5-[3-[[(4S)-1-[[3-[[4-[1-(2,6-dioxo-3-piperidyl)-4-fluoro-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (152)

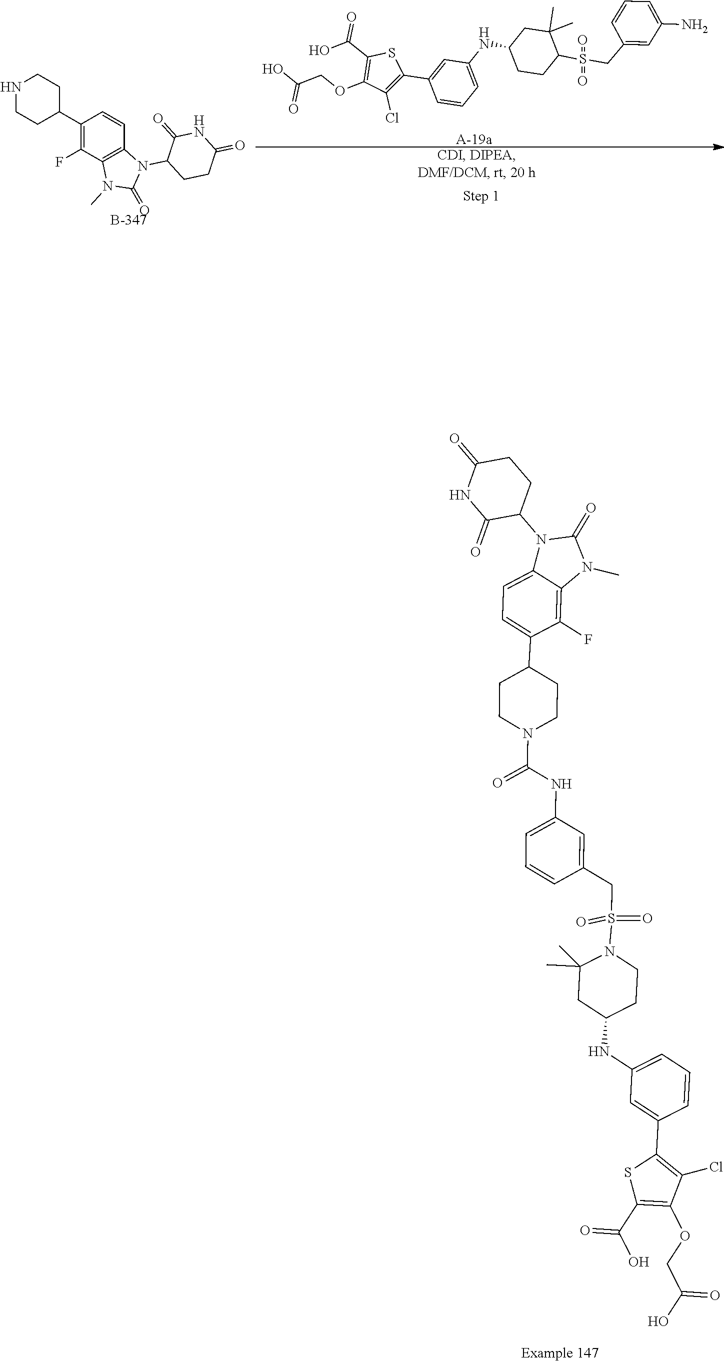 [SMILES: Cn1c(=O)n(C2CCC(=O)NC2=O)c2ccc(C3CCNCC3)c(F)c21 ; CC1(C)C[C@@H](Nc2cccc(-c3sc(C(=O)O)c(OCC(=O)O)c3Cl)c2)CCC1S(=O)(=O)Cc1cccc(N)c1 ; Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)c(F)c21]

### Step 3-(carboxymethoxy)-4-chloro-5-[3-[[(4S)-1-[[3-[[4-[1-(2,6-dioxo-3-piperidyl)-4-fluoro-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (152)

3-(carboxymethoxy)-4-chloro-5-[3-[[(4S)-1-[[3-[[4-[1-(2,6-dioxo-3-piperidyl)-4-fluoro-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (152, 16 mg, 14.66 μmol, 5% yield, formic acid salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[4-fluoro-3-methyl-2-oxo-5-(4-piperidyl)benzimidazol-1-yl]piperidine-2,6-dione (B-347) in a similar fashion to Example 23, except using excess DIPEA as base and DMF/THF as the solvent. The residue was purified by reverse phase purification [Purification method: Column: Biotage Ultra C18 (30 g, 25 μm); Mobile phase A: 0.1% Formic acid in water; Mobile phase B: Acetonitrile]. LCMS (ES+): m/z 994.2 [M+H]⁺. ¹H NMR (400 MHz, DMSO-d6): δ 11.12 (s, 1H), 8.64 (s, 1H), 7.57 (s, 1H), 7.47 (d, J=8.00 Hz, 1H), 7.25 (t, J=8.00 Hz, 1H), 7.17 (t, J=8.00 Hz, 1H), 7.00-6.92 (m, 3H), 6.82 (s, 1H), 6.76 (d, J=7.60 Hz, 1H), 6.65 (d, J=8.00 Hz, 1H), 6.54 (s, 1H), 5.62 (d, J=8.40 Hz, 1H), 5.38-5.34 (m, 1H), 4.60 (s, 2H), 4.35-4.24 (m, 4H), 3.50-3.47 (m, 4H), 3.13-3.08 (m, 4H), 2.96-2.90 (m, 4H), 2.68-2.65 (m, 2H), 2.03-2.01 (m, 1H), 1.84-1.76 (m, 4H), 1.66-1.64 (m, 2H), 1.49 (s, 3H), 1.40 (s, 3H), 1.16-1.11 (m, 1H).

### Example 153

### 3-(carboxymethoxy)-4-chloro-5-[3-[[(4S)-1-[[3-[[4-[1-(2,6-dioxo-3-piperidyl)-6-fluoro-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (153)

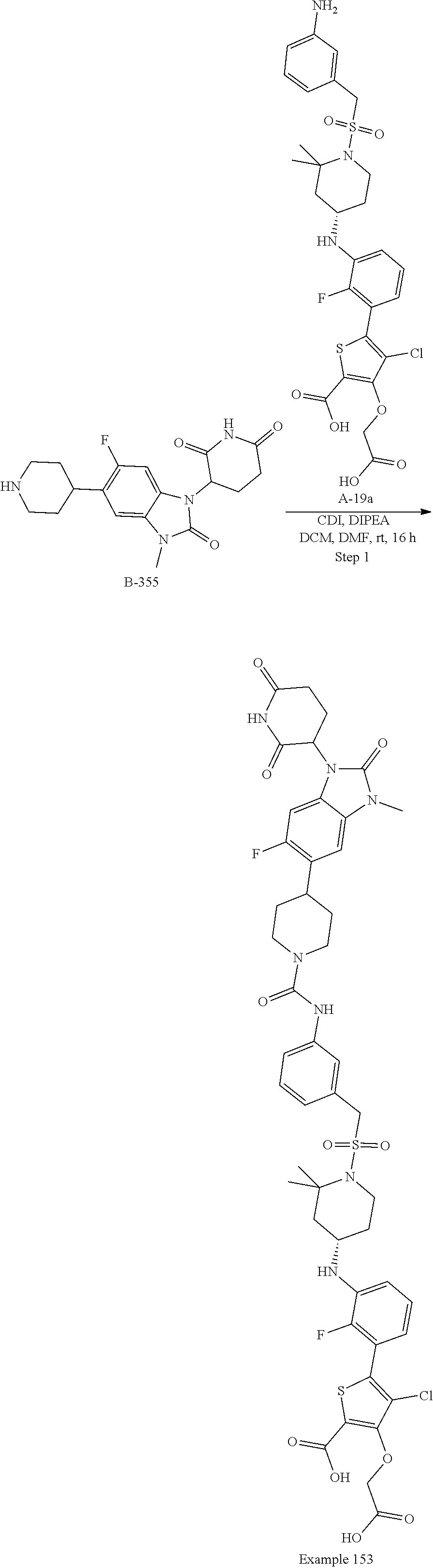

### Step 1: 3-(carboxymethoxy)-4-chloro-5-[3-[[(4S)-1-[[3-[[4-[1-(2,6-dioxo-3-piperidyl)-6-fluoro-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (153)

3-(carboxymethoxy)-4-chloro-5-[3-[[(4S)-1-[[3-[[4-[1-(2,6-dioxo-3-piperidyl)-6-fluoro-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (153, 25 mg, 22.11 μmol, 26% yield, TFA salt) was synthesized from 5-[3-[[(4S)-1-[(3-aminophenyl)methyl sulfonyl]-2,2-dimethyl-4-piperidyl]amino]phenyl]-3-(carboxymethoxy)-4-chloro-thiophene-2-carboxylic acid (A-19a) and 3-(6-fluoro-3-methyl-2-oxo-5-(piperidin-4-yl)-2,3-dihydro-1H-benzo[d]imidazol-1-yl)piperidine-2,6-dione (B-355) in a similar fashion to Example 23, except using excess DIPEA as base and DMF/THF as the solvent. The residue was purified by reverse phase prep HPLC [Purification method: XSelecta new-029, Mobile phase A: 0.1% TFA in water; Mobile phase B: MeCN]. LCMS (ES+): m/z 994.2 [M+H]⁺. ¹H NMR (400 MHz, DMSO-d₆): δ 13.10 (bs, 1H), 11.09 (s, 1H), 8.63 (s, 1H), 7.58 (s, 1H), 7.49 (d, J=9.20 Hz, 1H), 7.10-7.25 (m, 3H), 7.04-6.97 (m, 1H), 6.87 (s, 1H), 6.82-6.80 (m, 1H), 6.70-6.68 (m, 1H), 6.52 (bs, 1H), 5.70 (bs, 1H), 5.36-5.32 (m, 1H), 4.91 (s, 2H), 4.38-4.24 (m, 4H), 3.54-3.46 (m, 4H), 3.33 (s, 3H), 3.16-3.08 (m, 3H), 2.96-2.88 (m, 3H), 2.68-2.64 (m, 2H), 1.81-2.00 (m, 7H), 1.50 (s, 3H), 1.47-1.40 (m, 4H), 1.20-1.00 (m, 1H).

### Example 154

### 3-(carboxymethoxy)-4-chloro-5-[3-[[(4S)-1-[[3-[[4-[3-cyclopropyl-1-(2,6-dioxo-3-piperidyl)-2-oxo-benzimidazol-S-yl]piperidine-1-carbonyl]amino]phenyl]methylsulfonyl]-2,2-dimethyl-4-piperidyl]amino]phenyl]thiophene-2-carboxylic acid (154)

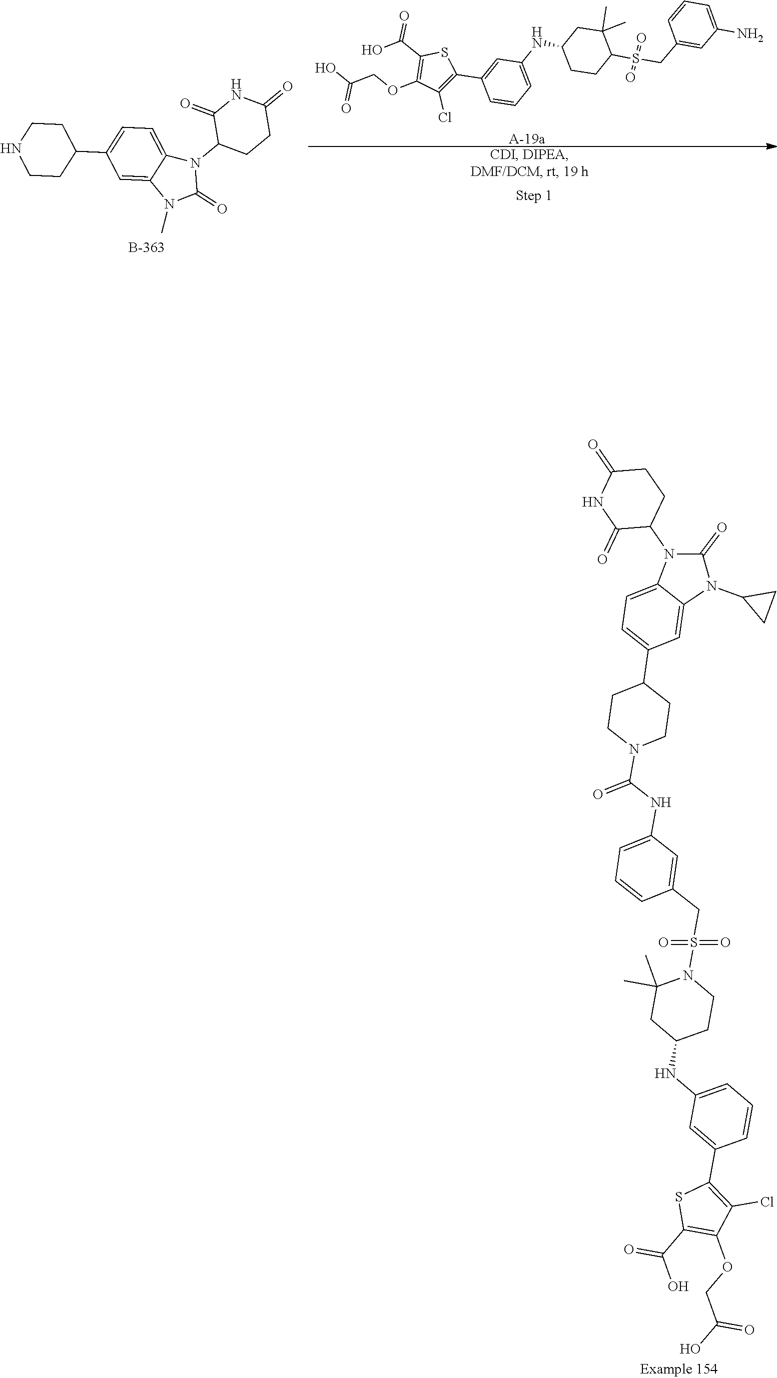 [SMILES: Cn1c(=O)n(C2CCC(=O)NC2=O)c2ccc(C3CCNCC3)cc21 ; CC1(C)C[C@@H](Nc2cccc(-c3sc(C(=O)O)c(OCC(=O)O)c3Cl)c2)CCC1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C3CC3)c(=O)n4C3CCC(=O)NC3=O)CC2)c1]

### Step 1: 3-(carboxymethoxy)-4-chloro-5-[3-[[(4S)-1-[[3-[[4-[3-cyclopropyl-1-(2,6-dioxo-3-piperidyl)-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (154)

3-(carboxymethoxy)-4-chloro-5-[3-[[(4S)-1-[3-[4-[3-cyclopropyl-1-(2,6-dioxo-3-piperidyl)-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (154, 28.3 mg, 25.22 μmol, 19% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[3-cyclopropyl-2-oxo-5-(4-piperidyl)benzimidazol-1-yl]piperidine-2,6-dione (B-363) in a similar fashion to Example 23, except using excess DIPEA as base and DMF/THF as the solvent. The residue was purified by reverse phase prep HPLC [Purification method: Column: Zorbax C18 (250×21.2 mm) 7 μm; Mobile phase A: 0.1% TFA in water, Mobile phase B: MeCN]. LCMS (ES+): m/z 1002.2 [M+H]⁺. ¹H NMR (400 MHz, DMSO-d₆) δ 11.07 (s, 1H), 8.62 (s, 1H), 7.55 (s, 1H), 7.46-7.40 (m, 1H), 7.27-7.16 (m, 2H), 7.11 (s, 1H), 6.98 (d, J=7.8 Hz, 2H), 6.92 (d, J=8.3 Hz, 1H), 6.87-6.84 (m, 1H), 6.80 (d, J=7.6 Hz, 1H), 6.71-6.65 (m, 1H), 5.27 (dd, J=12.6, 5.1 Hz, 1H), 4.88 (s, 2H), 4.38-4.19 (m, 5H), 3.58 (s, 303H), 3.11 (t, J=12.4 Hz, 2H), 2.93-2.73 (m, 5H), 2.69-2.57 (m, 2H), 2.02-1.93 (m, 1H), 1.88-1.72 (m, 4H), 1.66-1.51 (m, 2H), 1.47 (s, 3H), 1.43-1.35 (m, 4H), 1.12-1.00 (m, 3H), 0.89-0.82 (m, 2H).

### Example 155

### 3-(carboxymethoxy)-4-chloro-5-[3-[[(4S)-1-[[3-[[4-[1-(2,6-dioxo-3-piperidyl)-2-oxo-3-(2,2,2-trifluoroethyl)benzimidazol-5-yl]piperidine-1-carbonyl]amino]phenyl]-methylsulfonyl]-2,2-dimethyl-4-piperidyl]amino]phenyl]thiophene-2-carboxylic acid (155)

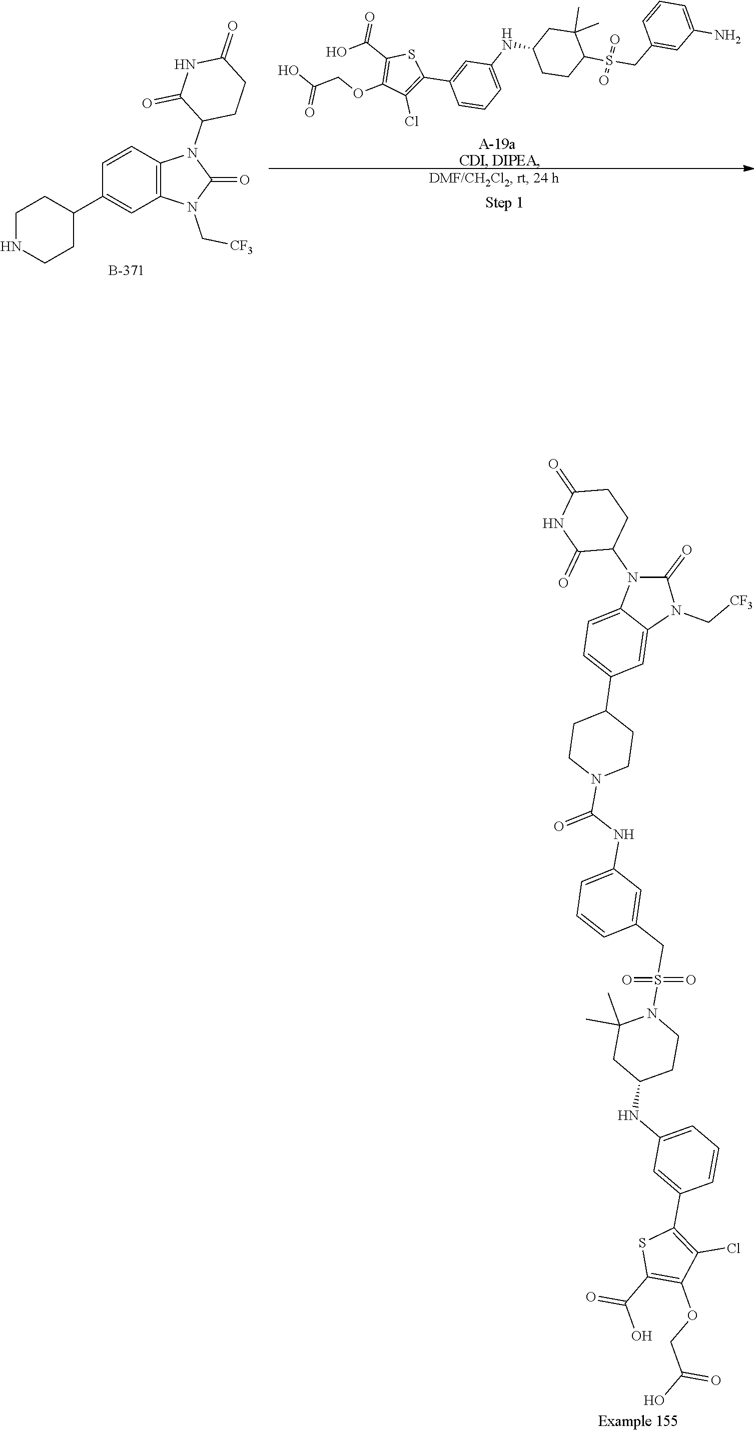 [SMILES: O=C1CCC(n2c(=O)n(CC(F)(F)F)c3cc(C4CCNCC4)ccc32)C(=O)N1 ; CC1(C)C[C@@H](Nc2cccc(-c3sc(C(=O)O)c(OCC(=O)O)c3Cl)c2)CCC1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(CC(F)(F)F)c(=O)n4C3CCC(=O)NC3=O)CC2)c1]

### Step 1: 3-(carboxymethoxy)-4-chloro-5-[3-[[(4S)-1-[[3-[[4-[1-(2,6-dioxo-3-piperidyl)-2-oxo-3-(2,2,2-trifluoroethyl)benzimidazol-5-yl]piperidine-1-carbonyl]amino]phenyl]-methylsulfonyl]-2,2-dimethyl-4-piperidyl]amino]phenyl]thiophene-2-carboxylic acid (155)

3-(carboxymethoxy)-4-chloro-5-[3-[[(4S)-1-[[3-[[4-[1-(2,6-dioxo-3-piperidyl)-2-oxo-3-(2,2,2-trifluoroethyl)benzi-midazol-5-yl]piperidine-1-carbonyl]amino]phenyl]methylsulfonyl]-2,2-dimethyl-4-piperidyl]amino]phenyl]thiophene-2-carboxylic acid (155, 48.98 mg, 41.14 μmol, 34% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[2-oxo-5-(4-piperidyl)-3-(2,2,2-trifluoroethyl)benzimidazol-1-yl]piperidine-2,6-dione (B-371) in a similar fashion to Example 23, except using excess DIPEA as base and DMF/THF as the solvent. The residue was purified by reverse phase prep HPLC [Purification method: Column: XSelect-C18 (19×150 mm), 5 μm; Mobile phase A: 0.1% TFA in Water: Mobile phase B: Acetonitrile], LCMS (ES+); m/z 1044.4 [M+H]⁺. ¹H NMR (400 MHz, DMSO-d6): δ 11.13 (s, 1H), 8.63 (s, 1H), 7.58 (s, 1H), 7.48 (d, J=8.40 Hz, 1H), 7.33 (s, 1H), 7.25 (t, J=8.00 Hz, 1H), 7.20 (t, J=8.00 Hz, 1H), 7.09 (d, J=3.60 Hz, 1H), 7.02-6.96 (m, 2H), 6.88 (s, 1H), 6.82 (d, J=7.60 Hz, 1H), 6.70 (d, J=6.80 Hz, 1H), 5.39 (dd, J=5.60, 12.80 Hz, 1H), 4.91 (s, 1H), 4.81-4.79 (m, 2H), 4.38-4.24 (m, 4H), 3.57-3.46 (m, 4H), 3.13 (t, J=8.40 Hz, 1H), 2.88 (t, J=5.60 Hz, 1H), 2.84-2.65 (m, 3H), 2.10-2.01 (m, 1H), 1.92-1.95 (m, 4H), 1.70-1.55 (m, 2H), 1.52-1.35 (m, 7H), 1.20-1.05 (m, 1H).

### Example 156

### 3-(carboxymethoxy)-4-chloro-5-(3-(((4S)-1-((3-(3-(3-(1-(2,6-dioxopiperidin-3-yl)-2-oxo-1,2-dihydrobenzo[cd]indol-6-yl)propyl)ureido)benzyl)sulfonyl)-2,2-dimethylpiperidin-4-yl)amino)phenyl)thiophene-2-carboxylic acid (156)

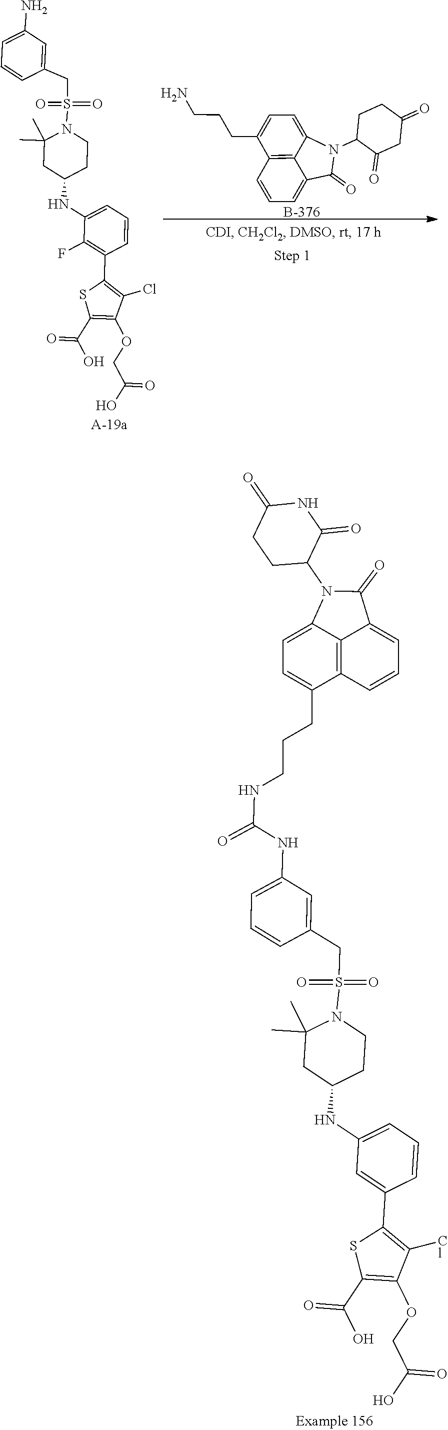 [SMILES: CC1(C)C[C@@H](Nc2cccc(-c3sc(C(=O)O)c(OCC(=O)O)c3Cl)c2F)CCN1S(=O)(=O)Cc1cccc(N)c1 ; NCCCc1ccc2c3c(cccc13)C(=O)N2C1CCC(=O)CC1=O ; CC1(C)C[C@@H](Nc2cccc(-c3sc(C(=O)O)c(OCC(=O)O)c3Cl)c2)CCN1S(=O)(=O)Cc1cccc(NC(=O)NCCCc2ccc3c4c(cccc24)C(=O)N3C2CCC(=O)NC2=O)c1]

### Step 1: 3-(carboxymethoxy)-4-chloro-5-(3-(((4S)-1-((3-(3-(3-(1-(2,6-dioxopiperidin-3-yl)-2-oxo-1,2-dihydrobenzo[cd]indol-6-yl)propyl)ureido)benzyl)sulfonyl)-2,2-dimethylpiperidin-4-yl)amino)phenyl)thiophene-2-carboxylic acid (156)

3-(carboxymethoxy)-4-chloro-5-(3-(((4S)-1-((3-(3-(3-(1-(2,6-dioxopiperidin-3-yl)-2-oxo-1,2-dihydrobenzo[cd]indol-6-yl)propyl)ureido)benzyl)sulfonyl)-2,2-dimethylpiperidin-4-yl)amino)phenyl)thiophene-2-carboxylic acid (156, 38 mg, 10% yield, TFA salt) was synthesized from 5-[3-[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a) and 3-[6-(3-aminopropyl)-2-oxo-benzo[cd]indol-1-yl]piperidine-2,6-dione (B-376) in a similar fashion to Example 23, except using excess DIPEA as base and DCM/DMSO as the solvent. The residue was purified by reverse phase prep HPLC [Purification method: Column-Zorbax, C18 (250×21 mm), Mobile phase A: 0.1% TFA in water; Mobile phase B: MeCN). LCMS (ES+): m/z 971.0 [M+H]⁺. ¹H NMR (400 MHz, DMSO-d₆) δ 11.13 (s, 1H), 8.56 (s, 1H), 8.33 (d, J=8.3 Hz, 1H), 8.09 (d, J=7.0 Hz, 1H), 7.84 (dd, J=8.3, 7.0 Hz, 1H), 7.47-7.44 (m, 1H), 7.42-7.38 (m, 1H), 7.34 (d, J=7.4 Hz, 1H), 7.20 (dt, J=17.2, 7.8 Hz, 2H), 7.06 (d, J=7.3 Hz, 1H), 6.95-6.91 (m, 1H), 6.87-6.84 (m, 1H), 6.80 (d, J=7.6 Hz, 1H), 6.71-6.66 (m, 1H), 6.32-6.22 (m, 1H), 5.43 (dd, J=13.0, 5.3 Hz, 1H), 4.91 (s, 2H), 4.36 (d, J=13.7 Hz, 1H), 4.24 (d, J=13.7 Hz, 1H), 3.47-3.38 (m, 2H), 3.24-3.16 (m, 2H), 3.15-3.00 (m, 2H), 2.99-2.90 (m, 1H), 2.81-2.70 (m, 1H), 2.13-2.04 (m, 1H), 1.89-1.73 (m, 4H), 1.44 (s, 3H), 1.42-1.37 (m, 4H), 1.12 (d, J=11.0 Hz, 1H).

### Example 157

### 3-(carboxymethoxy)-4-chloro-5-[3-[[1-[[3-[3-[3-(2,6-dioxo-3-piperidyl)-1-methyl-indazol-6-yl]propanoylamino]phenyl]methylsulfonyl]-4-piperidyl]amino]phenyl]thiophene-2-carboxylic acid (157)

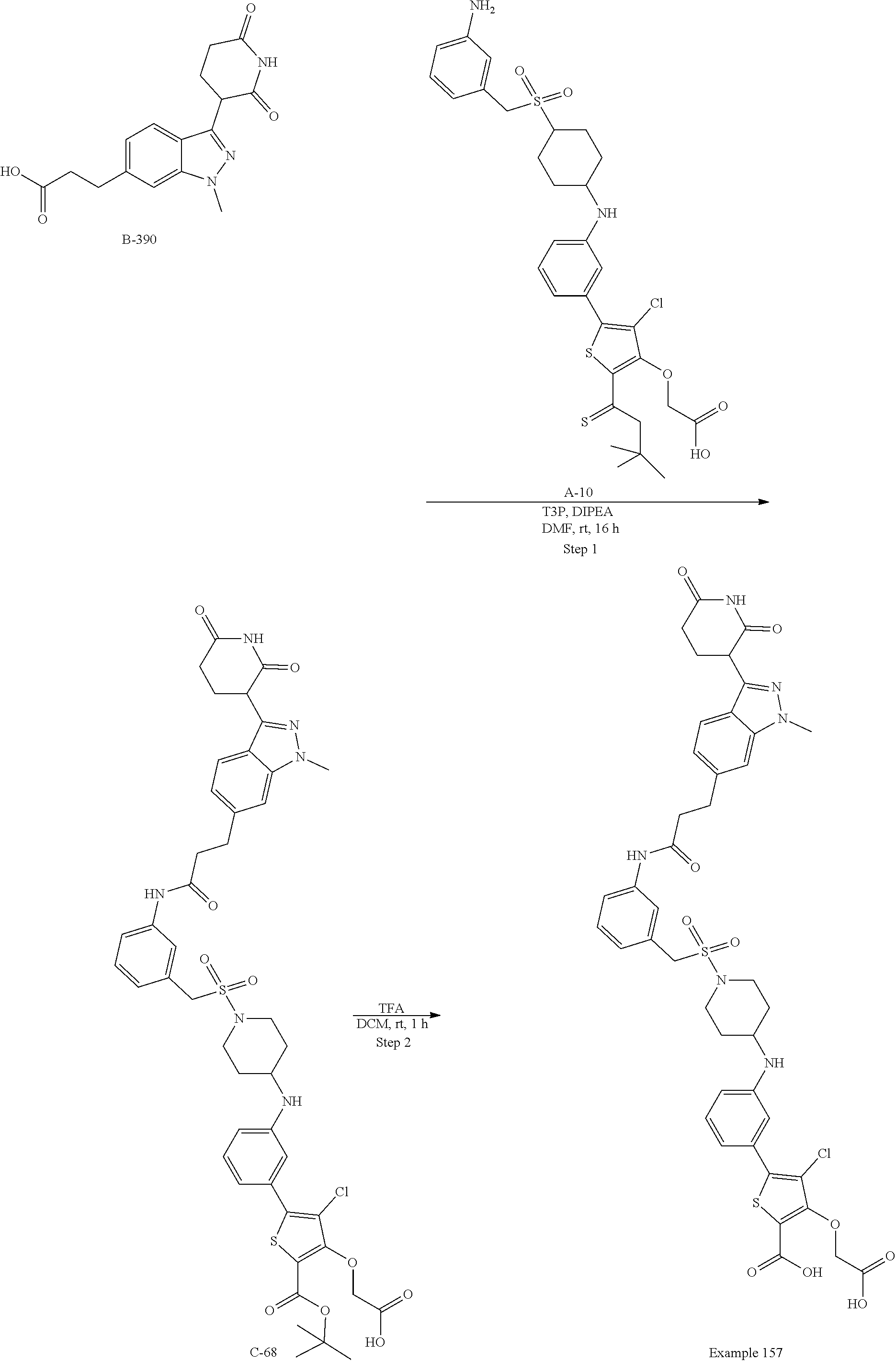

### Step 2-[[2-tert-butoxycarbonyl-4-chloro-5-[3-[[1-[[3-[3-[3-(2,6-dioxo-3-piperidyl)-1-methyl-indazol-6-yl]propanoylamino]phenyl]methylsulfonyl]-4-piperidyl]amino]phenyl]-3-thienyl]oxy]acetic acid (C-68)

2-[[2-tert-butoxycarbonyl-4-chloro-5-[3-[[1-[[3-[3-[3-(2,6-dioxo-3-piperidyl)-1-methyl-indazol-6-yl]propanoylamino]phenyl]methylsulfonyl]-4-piperidyl]amino]phenyl]-3-thienyl]oxy]acetic acid (C-68, 15 mg, 15.23 μmol, 10% yield) was synthesized from 2-[[5-[3-[[1-[(3-aminophenyl)methylsulfonyl]-4-piperidyl]amino]phenyl]-2-tert-butoxycarbonyl-4-chloro-3-thienyl]oxy]acetic acid (A-10) and 3-[3-(2,6-dioxo-3-piperidyl)-1-methyl-indazol-6-yl]propionic acid (B-390) in a similar fashion to Compound C-6, except using DIPEA as base. The residue was purified by reverse phase prep HPLC (Column: XBridge-C18, 150 mm; 0.1% TFA in water/MeCN). LCMS (ES+): m/z 933.2 [M+H]⁺.

### Step 2: 3-(carboxymethoxy)-4-chloro-5-[3-[[1-[[3-[3-[3-(2,6-dioxo-3-piperidyl)-1-methyl-indazol-6-yl]propanoylamino]phenyl]methylsulfonyl]-4-piperidyl]amino]phenyl]thiophene-2-carboxylic acid (157)

3-(carboxymethoxy)-4-chloro-5-[3-[[1-[[3-[3-[3-(2,6-dioxo-3-piperidyl)-1-methyl-indazol-6-yl]propanoylamino]phenyl]methylsulfonyl]-4-piperidyl]amino]phenyl]thiophene-2-carboxylic acid (157, 0.0065 g, 6.90 μmol, 43% yield) was synthesized from 2-[[2-tert-butoxycarbonyl-4-chloro-5-[3-[[1-[[3-[3-[3-(2,6-dioxo-3-piperidyl)-1-methyl-indazol-6-yl]propanoylamino]phenyl]methylsulfonyl]-4-piperidyl]amino]phenyl]-3-thienyl]oxy]acetic acid (C-68) in a similar fashion to Compound C-1. The residue was triturated with diethyl ether. LCMS (ES+): m/z 877.1 [M+H]⁺. ¹H NMR (400 MHz, DMSO-d₆) δ 10.88 (s, 1H), 10.04 (s, 1H), 7.66 (s, 1H), 7.61 (d, J=8.3 Hz, 1H), 7.59-7.54 (m, 1H), 7.43 (s, 1H), 7.30 (t, J=7.9 Hz, 1H), 7.20 (t, J=7.9 Hz, 1H), 7.08 (d, J=7.4 Hz, 1H), 7.03 (d, J=8.4 Hz, 1H), 6.89-6.85 (m, 1H), 6.80 (d, J=7.6 Hz, 1H), 6.73-6.68 (m, 1H), 5.91-5.85 (m, 1H), 4.86 (s, 2H), 4.36 (s, 2H), 4.32 (dd, J=9.7, 5.2 Hz, 1H), 3.94 (s, 3H), 3.59-3.50 (m, 3H), 3.06 (t, J=7.6 Hz, 2H), 3.01-2.92 (m, 2H), 2.75-2.68 (m, 2H), 2.65-2.58 (m, 3H), 2.46 (s, 13H), 2.21-2.10 (m, 1H), 1.99-1.91 (m, 2H), 1.44-1.30 (m, 2H).

### Example 158

### 3-(carboxymethoxy)-4-chloro-5-(3-(((4S)-1-((3-(4-((1-(2,6-dioxopiperidin-3-yl)-2-oxo-1,2-dihydrobenzo[cd]indol-6-yl)methyl)piperidine-1-carboxamido)benzyl)sulfonyl)-2,2-dimethylpiperidin-4-yl)amino)phenyl)thiophene-2-carboxylic acid (158)

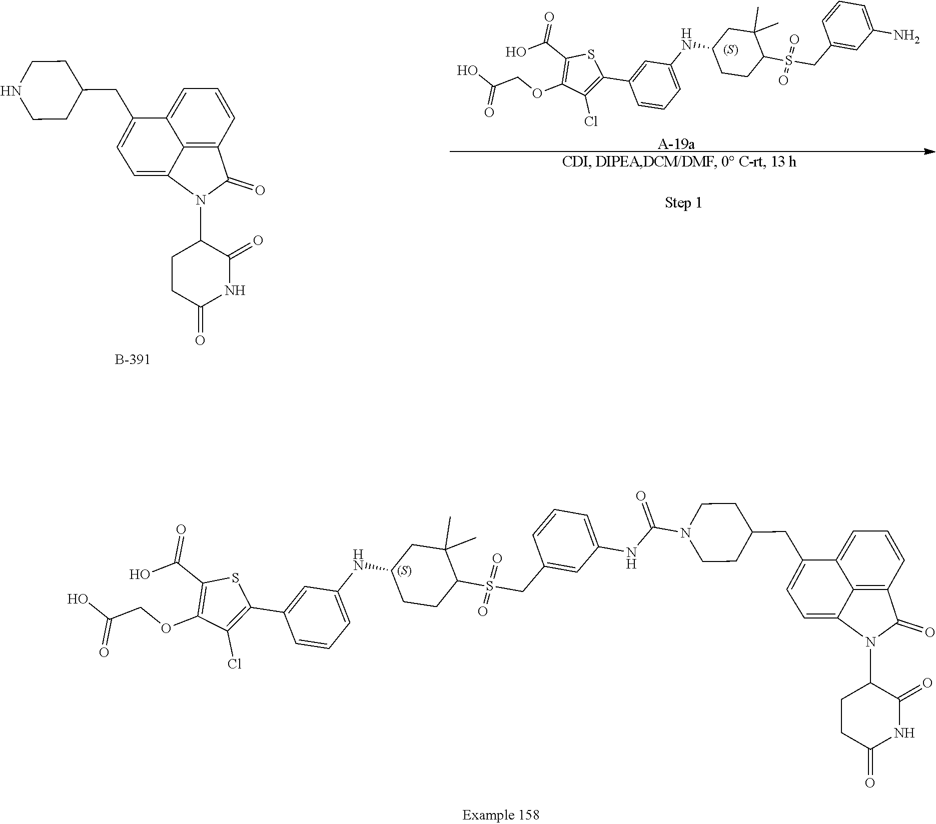

### Step 1: 3-(carboxymethoxy)-4-chloro-5-(3-(((4S)-1-((3-(4-((1-(2,6-dioxopiperidin-3-yl)-2-oxo-1,2-dihydrobenzo[cd]indol-6-yl)methyl)piperidine-1-carboxamido)benzyl)sulfonyl)-2,2-dimethylpiperidin-4-yl)amino)phenyl)thiophene-2-carboxylic acid (158)

3-(carboxymethoxy)-4-chloro-5-(3-(((4S)-1-((3-(4-((1-(2,6-dioxopiperidin-3-yl)-2-oxo-1,2-dihydrobenzo[cd]indol-6-yl)methyl)piperidine-1-carboxamido)benzyl)sulfonyl)-2,2-dimethylpiperidin-4-yl)amino)phenyl)thiophene-2-carboxylic acid (158, 90.94 mg, 77.56 μmol, 19% yield, TFA salt) was synthesized from(S)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19a, 249.78 mg, 410.73 μmol) and 3-(2-oxo-6-(piperidin-4-ylmethyl)benzo[cd]indol-[(2H)-yl)piperidine-2,6-dione (B-391, 170 mg, 410.73 μmol, 1 eq, HCl salt) in a similar fashion to Compound B-242, except using DCM/DMF as the solvent. The resulting mixture was purified by reversed phase column (0.1% FA in water/acetonitrile) and then purified by prep-HPLC (flow: 25 mL/min; gradient: from 48-78% water (0.1% TFA) in MeCN over 10 min; column: Phenomenex Synergi C18 150×25 mm×10 μm) and lyophilized.

LCMS (ESI): m/z 1011.5 [M+H]⁺

¹H NMR (400 MHz, DMSO-d₆) δ=11.12 (s, 1H), 8.53 (s, 1H), 8.38 (d, J=8.2 Hz, 1H), 8.10 (d, J=6.8 Hz, 1H), 7.85 (dd, J=7.2, 8.1 Hz, 1H), 7.57-7.50 (m, 1H), 7.42 (br d, J=8.1 Hz, 1H), 7.31 (d, J=7.3 Hz, 1H), 7.20 (td, J=7.8, 12.4 Hz, 2H), 7.09 (d, J=7.2 Hz, 1H), 6.96 (br d, J=7.7 Hz, 1H), 6.86 (s, 1H), 6.81 (d, J=7.5 Hz, 1H), 6.68 (dd, J=1.4, 8.3 Hz, 1H), 5.51-5.38 (m, 1H), 4.91 (s, 2H), 4.34 (br d, J=13.7 Hz, 1H), 4.23 (br d, J=13.7 Hz, 1H), 4.12 (br d, J=12.8 Hz, 2H), 3.56-3.39 (m, 2H), 3.18-3.05 (m, 1H), 2.96 (br d, J=5.7 Hz, 2H), 2.83-2.66 (m, 3H), 2.09 (br dd, J=5.4, 9.7 Hz, 1H), 1.90-1.73 (m, 3H), 1.68-1.56 (m, 2H), 1.52-1.33 (m, 7H), 1.29-1.16 (m, 2H), 1.15-1.02 (m, 2H).

### Example 159

### 3-(carboxymethoxy)-4-chloro-5-(3-(((4S)-1-((3-(4-(1-(2,6-dioxopiperidin-3-yl)-2-oxo-3-(tetrahydro-2H-pyran-4-yl)-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (159)

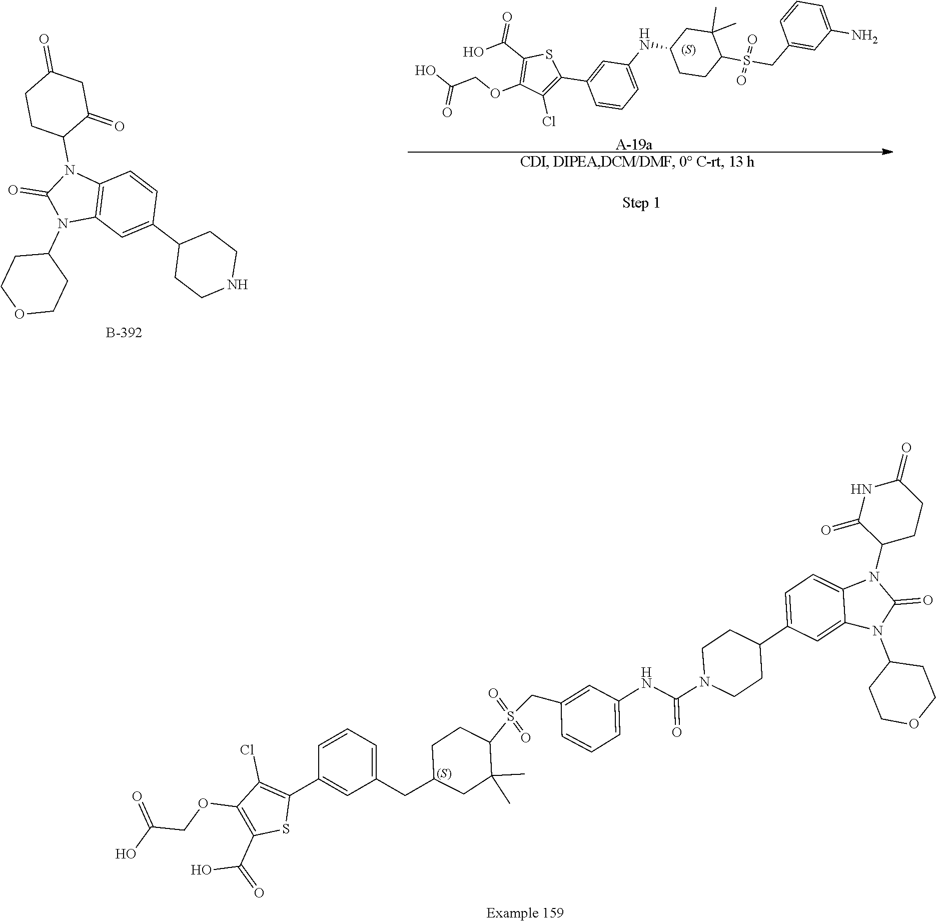

### Step 1: 3-(carboxymethoxy)-4-chloro-5-(3-(4S)-1-(3-(4-(1-(2,6-dioxopiperidin-3-yl)-2-oxo-3-(tetrahydro-2H-pyran-4-yl)-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (159)

3-(carboxymethoxy)-4-chloro-5-(3-(((4S)-1-((3-(4-(1-(2,6-dioxopiperidin-3-yl)-2-oxo-3-(tetrahydro-2H-pyran-4-yl)-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (159, 83.9 mg, 71.57 μmol, 31% yield, TFA salt) was synthesized from(S)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19a, 138.60 mg, 227.92 μmol) and 3-[2-oxo-5-(4-piperidyl)-3-tetrahydropyran-4-yl-benzimidazol-1-yl]piperidine-2,6-dione (B-392, 120 mg, 227.92 μmol, 1 eq. TFA salt) in a similar fashion to Compound B-242, except using DCM/DMF as the solvent. The residue was purified by prep-HPLC (flow: 25 mL/min; gradient: from 68%-38% water (0.1% TFA)-ACN; column: 3_Phenomenex Luna C18 75×30 mm×3 μm) and lyophilized. LCMS (ESI): m/z 1047.2 [M+H]⁺

¹H NMR (400 MHz, DMSO-d₆) δ=11.10-11.05 (m, 1H), 8.66-8.58 (m, 1H), 7.61-7.54 (m, 1H), 7.51-7.42 (m, 1H), 7.28-7.17 (m, 3H), 7.04-6.96 (m, 2H), 6.94-6.80 (m, 3H), 6.72-6.66 (m, 1H), 5.37-5.28 (m, 1H), 4.93-4.87 (m, 2H), 4.49-4.42 (m, 1H), 4.37-4.23 (m, 4H), 3.98 (br d, J=7.2 Hz, 2H), 3.14 (br d, J=5.9 Hz, 4H), 2.91 (br s, 1H), 2.88 (br s, 4H), 2.68-2.58 (m, 2H), 2.46-2.39 (m, 2H), 2.03-1.96 (m, 1H), 1.88-1.76 (m, 4H), 1.69-1.60 (m, 4H), 1.50-1.38 (m, 7H), 1.16-1.06 (m, 1H).

### Example 160

### 3-(carboxymethoxy)-4-chloro-5-(3-(((4S)-1-((3-(4-(1-(2,6-dioxopiperidin-3-yl)-3-(2-methoxyethyl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (160)

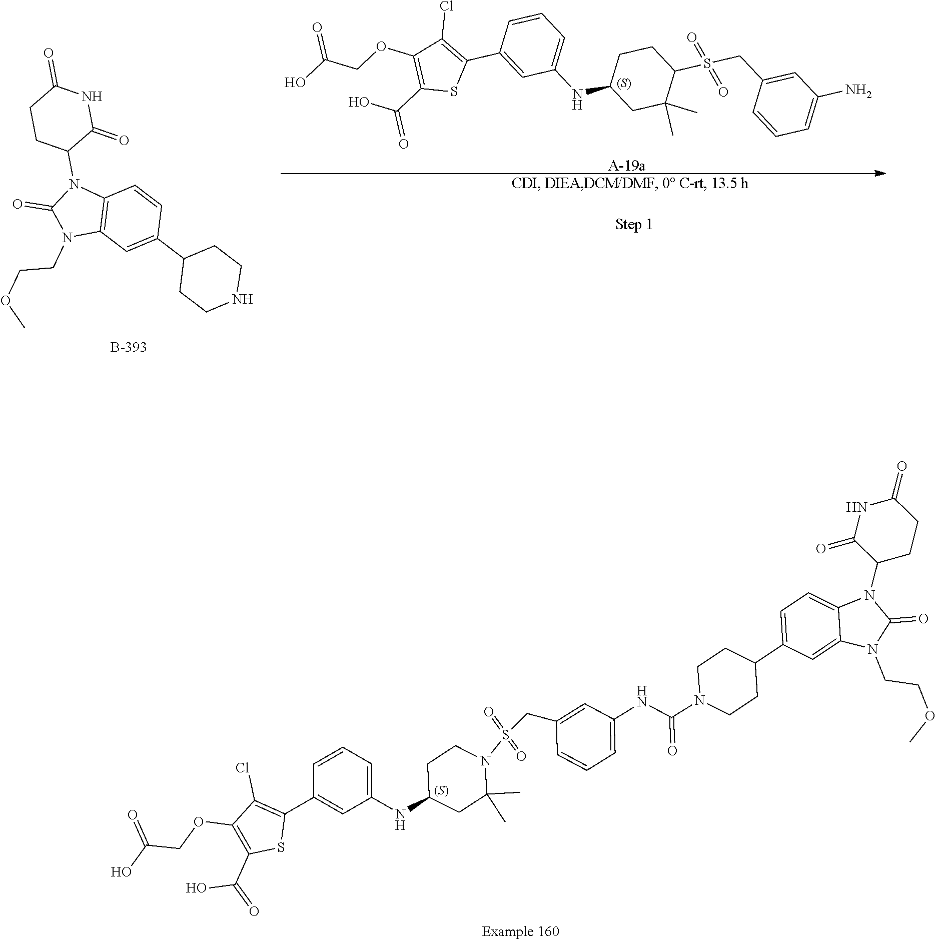

### Step 1: 3-(carboxymethoxy)-4-chloro-5-(3-(((4S)-1-((3-(4-(1-(2,6-dioxopiperidin-3-yl)-3-(2-methoxyethyl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (160)

3-(carboxymethoxy)-4-chloro-5-(3-(((4S)-1-((3-(4-(1-(2,6-dioxopiperidin-3-yl)-3-(2-methoxyethyl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (160, 138.54 mg, 120.89 μmol, 30% yield, TFA salt) was synthesized from(S)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19a, 243.02 mg, 399.63 μmol) and 3-[3-(2-methoxyethyl)-2-oxo-5-(4-piperidyl)benzimidazol-1-yl]piperidine-2,6-dione (B-393, 200 mg, 399.63 μmol, TFA salt) in a similar fashion to Compound B-242, except using DCM/DMF as the solvent. The residue was purified by prep-HPLC (flow: 25 mL/min; gradient: from 39-69% water (0.1% TFA) in MeCN over 10 min; column; Phenomenex Synergi C18 150×25 mm×10 μm) and lyophilized. LCMS (ESI): m/z 1020.4 [M+H]⁺

¹H NMR (400 MHz, DMSO-d₆) δ=11.10 (s, 1H), 8.63 (s, 1H), 7.58 (s, 1H), 7.48 (d, J=8.4 Hz, 1H), 7.28-7.17 (m, 3H), 7.03-6.97 (m, 2H), 6.91 (s, 1H), 6.88 (s, 1H), 6.82 (d, J=7.6 Hz, 1H), 6.70 (br d, J=8.8 Hz, 1H), 5.34 (dd, J=5.2, 12.8 Hz, 1H), 4.92 (s, 2H), 4.40-4.26 (m, 4H), 4.02-3.99 (m, 2H), 3.60 (t, J=5.6 Hz, 2H), 3.56-3.42 (m, 2H), 3.24 (s, 3H), 3.13 (t, J=12.0 Hz, 1H), 2.97-2.83 (m, 3H), 2.82-2.58 (m, 3H), 2.05-1.96 (m, 1H), 1.90-1.74 (m, 4H), 1.70-1.56 (m, 2H), 1.53-1.36 (m, 7H), 1.18-1.04 (m, 1H).

### Example 161

### 3-(carboxymethoxy)-4-chloro-5-[3-[[(4S)-1-[[5-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (161)

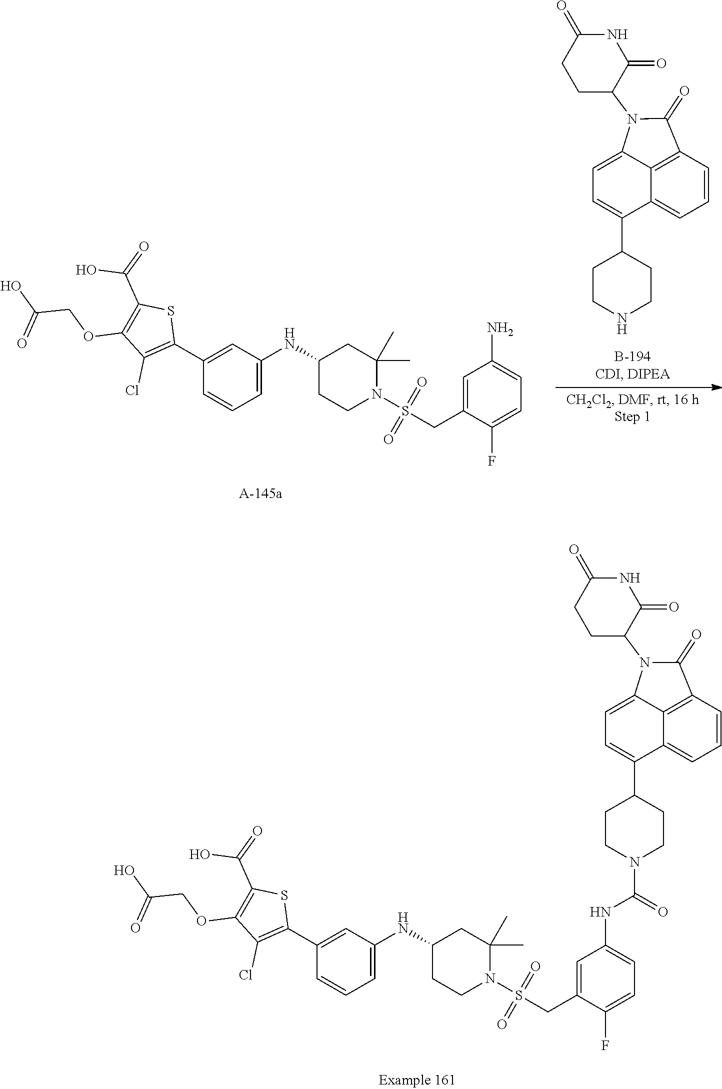 [SMILES: CC1(C)C[C@@H](Nc2cccc(-c3sc(C(=O)O)c(OCC(=O)O)c3Cl)c2)CCN1S(=O)(=O)Cc1cc(N)ccc1F ; O=C1CCC(N2C(=O)c3cccc4c(C5CCNCC5)ccc2c34)C(=O)N1 ; CC1(C)C[C@@H](Nc2cccc(-c3sc(C(=O)O)c(OCC(=O)O)c3Cl)c2)CCN1S(=O)(=O)Cc1cc(NC(=O)N2CCC(c3ccc4c5c(cccc35)C(=O)N4C3CCC(=O)NC3=O)CC2)ccc1F]

### Step 1: 3-(carboxymethoxy)-4-chloro-5-[3-[[(4S)-1-[[5-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (161)

3-(carboxymethoxy)-4-chloro-5-[3-[[(4S)-1-[[5-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (161, 40 mg, 35.46 μmol, 8% yield, formic acid salt) was synthesized from 5-[3-[[(4S)-1-[(5-amino-2-fluoro-phenyl)methylsulfonyl]-2,2-dimethyl-4-piperidyl]amino]phenyl]-3-(carboxymethoxy)-4-chloro-thiophene-2-carboxylic acid (A-145a, 350 mg, 468.2 μmol, TFA salt) and 3-[2-oxo-6-(4-piperidyl)benzo[cd]indol-1-yl]piperidine-2,6-dione (B-194, 207 mg, 468.2 μmol, TFA salt) in a similar fashion to Compound B-242. The residue was purified by reverse-phase preparative HPLC [column Select C18 (250×19) mm, 5 microns; Mobile Phase A: 0.1% FA in water and Mobile Phase B: MeCN, flow rate: 15 mL/min].

¹H-NMR (400 MHz, DMSO-d6): δ 11.13 (s, 1H), 8.70 (s, 1H), 8.51 (d, J=8.40 Hz, 1H), 8.12 (d, J=6.80 Hz, 1H), 7.88 (t, J=8.40 Hz, 1H), 7.65-7.64 (m, 1H), 7.54-7.53 (m, 1H), 7.39 (d, J=8.00 Hz, 2H), 7.20-7.09 (m, 4H), 6.85 (s, 1H), 6.78 (d, J=7.60 Hz, 1H), 6.68 (d, J=8.00 Hz, 1H), 5.70 (d, J=7.60 Hz, 1H), 5.45 (d, J=7.60 Hz, 1H), 4.70 (s, 2H), 4.43-4.31 (m, 5H), 3.18-2.92 (m, 6H), 2.81-2.61 (m, 2H), 2.10-2.08 (m, 1H), 1.93-1.68 (m, 6H), 1.54-1.38 (m, 7H), 1.20-1.08 (m, 1H). LCMS (ES+): m/z 1015.0 [M+H]⁺

### Example 162

### 3-(carboxymethoxy)-4-chloro-5-[3-[[(4R)-1-[5-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (162)

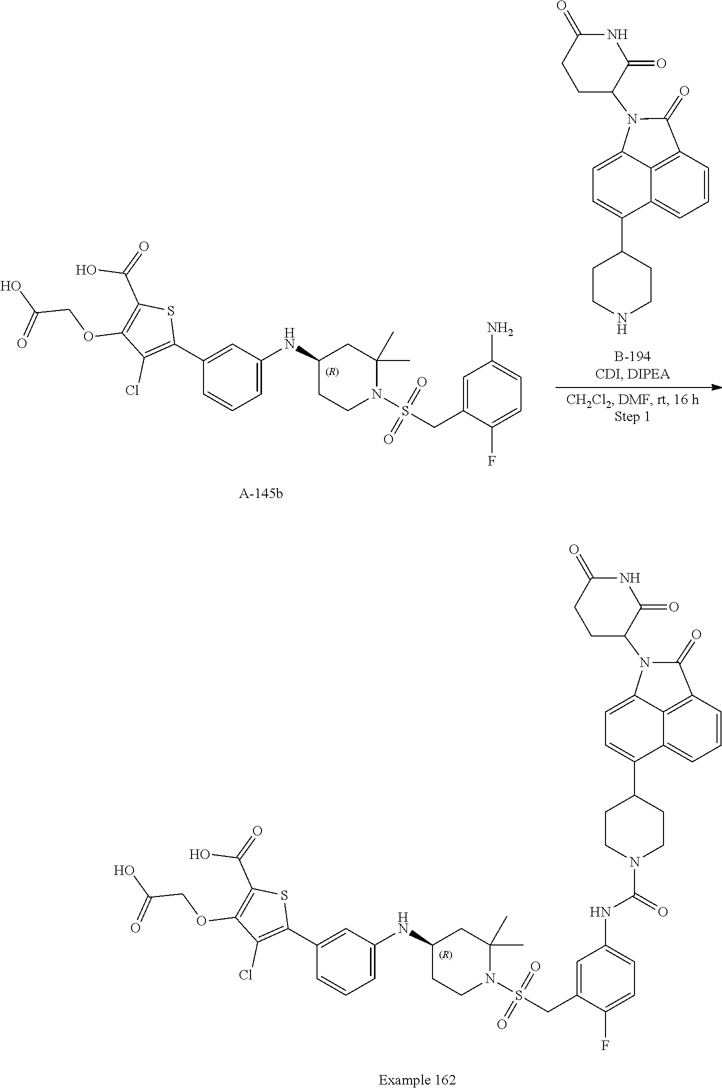

### Step 1: 3-(carboxymethoxy)-4-chloro-5-[3-[[(4R)-1-[[5-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (162)

3-(carboxymethoxy)-4-chloro-5-[3-[(4R)-1-[[5-[[4-[1-(2,6-dioxo-3-piperidyl)-2-oxo-benzo[cd]indol-6-yl]piperidine-1-carbonyl]amino]-2-fluoro-phenyl]methylsulfonyl]-2,2-dimethyl-4-piperidyl]amino]phenyl]thiophene-2-carboxylic acid (162, 39 mg, 33.80 μmol, 8% yield, formic acid salt) was synthesized from 5-[3-[(4R)-1-[(5-amino-2-fluoro-phenyl)methylsulfonyl]-2,2-dimethyl-4-piperidyl]amino]phenyl]-3-(carboxymethoxy)-4-chloro-thiophene-2-carboxylic acid (A-145b, 350 mg, 472.9 μmol, TFA salt) and 3-[2-oxo-6-(4-piperidyl)benzo[cd]indol-1-yl]piperidine-2,6-dione (B-194, 210 mg, 472.9 μmol, TFA salt) in a similar fashion to Compound B-242. The residue was purified by reverse-phase preparative HPLC [column Select C18 (250×19) mm, 5 microns; Mobile Phase A: 0.1% FA in water and Mobile Phase B: MeCN, flow rate: 15 mL/min] and lyophilized.

¹H-NMR (400 MHz, DMSO-d6): δ 11.12 (s, 1H), 8.71 (s, 1H), 8.51 (d, J=8.40 Hz, 1H), 8.42 (s, 1H), 8.12 (d, J=6.80 Hz, 1H), 7.89 (t, J=7.20 Hz, 1H), 7.67-7.64 (m, 1H), 7.56-7.52 (m, 1H), 7.39 (d, J=7.60 Hz, 1H), 7.18-7.09 (m, 3H), 6.83 (s, 1H), 6.76 (d, J=8.00 Hz, 1H), 6.65 (d, J=8.40 Hz, 1H), 5.66 (d, J=7.60 Hz, 1H), 5.44 (dd, J=5.20, 12.80 Hz, 1H), 4.42-4.31 (m, 6H), 3.62-3.55 (m, 4H), 3.46-2.96 (m, 4H), 2.68-2.55 (m, 2H), 2.20-2.08 (m, 1H), 1.93-1.71 (m, 6H), 1.51-1.42 (m, 7H), 1.20-1.08 (m, 1H). LCMS (ES+): m/z 1015.0 [M+H]⁺.

### Example 163

### 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)-1,4-diazepane-1-carboxamido)benzyl)sulfonyl)-2,2-dimethylpiperidin-4-yl)amino)phenyl)thiophene-2-carboxylic acid (163)

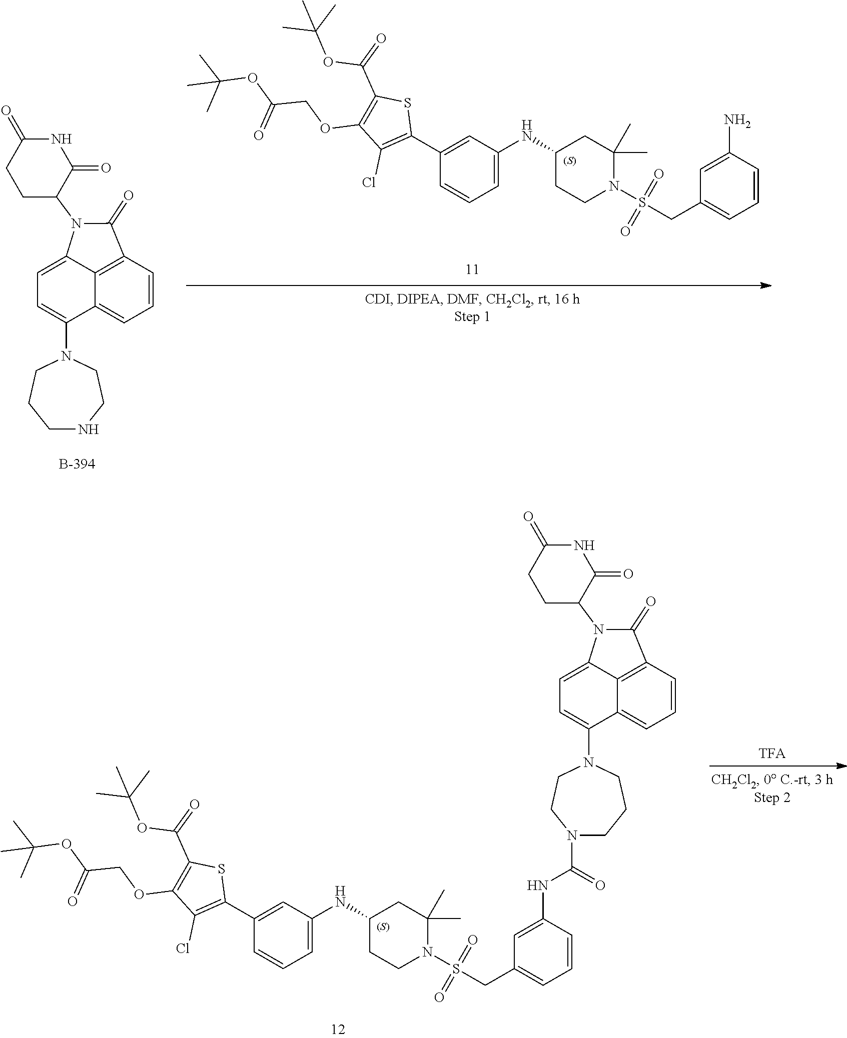

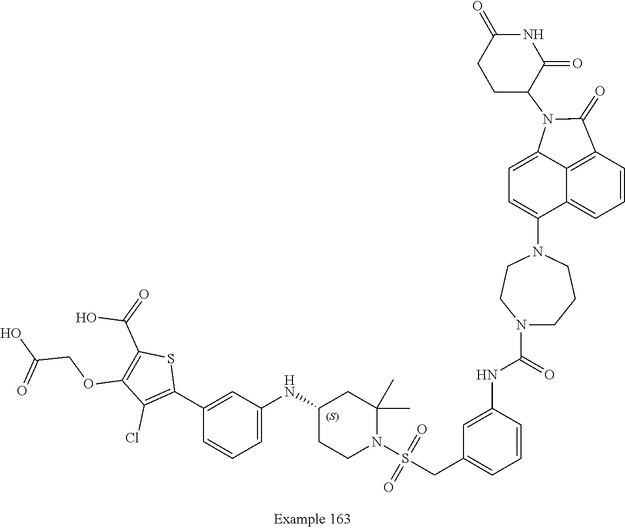

### Step 1: tert-butyl 3-(2-(tert-butoxy)-2-oxoethoxy)-4-chloro-5-(3-(((4S)-1-((3-(4-(1-(2,6-dioxopiperidin-3-yl)-2-oxo-1,2-dihydrobenzo[cd]indol-6-yl)-1,4-diazepane-1-carboxamido)benzyl)sulfonyl)-2,2-dimethylpiperidin-4-yl)amino)phenyl)thiophene-2-carboxylate (12)

tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]-1,4-diazepane-1-carbonyl]amino]phenyl]methylsulfonyl]-2,2-dimethyl-4-piperidyl]amino]phenyl]thiophene-2-carboxylate (12, 250 mg, 58.46 μmol, 29% yield, 26% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (11, 216.56 mg, 300.64 μmol) and 3-[6-(1,4-diazepan-1-yl)-2-oxo-benzo[cd]indol-1-yl]piperidine-2,6-dione (B-394, 105 mg, 200.43 μmol, TFA salt) in a similar fashion to Compound B-242 and was used without further purification in the next reaction. LCMS (ES+): m/z 1124.3 [M+H]⁺

### Step 2: 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)-1,4-diazepane-1-carboxamido)benzyl)sulfonyl)-2,2-dimethylpiperidin-4-yl)amino)phenyl)thiophene-2-carboxylic acid (163)

3-(carboxymethoxy)-4-chloro-5-[3-[[(4S)-1-[[3-[[4-[1-(2,6-dioxo-3-piperidyl)-2-oxo-benzo[cd]indol-6-yl]-1,4-diazepane-1-carbonyl]amino]phenyl]methylsulfonyl]-2,2-dimethyl-4-piperidyl]amino]phenyl]thiophene-2-carboxylic acid (163, 55 mg, 49.90 μmol, 86% yield, formic acid salt) was synthesized from tert-butyl 3-(2-tert-butoxy-2-oxo-ethoxy)-4-chloro-5-[3-[[(4S)-1-[3-[4-[1-(2,6-dioxo-3-piperidyl)-2-oxo-benzo[cd]indol-6-yl]-1,4-diazepane-1-carbonyl]amino]phenyl]methylsulfonyl]-2,2-dimethyl-4-piperidyl]amino]phenyl]thiophene-2-carboxylate (12, 250 mg, 57.79 μmol) in a similar fashion to Compound A-26. The residue was purified by reverse-phase preparative HPLC [Column: X-Select C18, (150×19) mm, 5 microns, Mobile phase A: 0.1% Formic acid in water and Mobile Phase B: MeCN].

¹H-NMR (400 MHz, DMSO-d6): δ 11.12 (s, 1H), 8.45 (d, J=11.60 Hz, 1H), 8.31 (d, J=8.40 Hz, 1H), 8.08 (d, J=7.20 Hz, 1H), 7.81 (t, J=7.20 Hz, 1H), 7.61 (s, 1H), 7.51 (d, J=8.40 Hz, 1H), 7.26 (t, J=8.00 Hz, 1H), 7.15 (t, J=8.00 Hz, 1H), 7.04-6.99 (m, 2H), 6.81 (s, 1H), 6.76 (d, J=8.00 Hz, 1H), 6.63 (d, J=7.60 Hz, 1H), 5.60 (d, J=8.40 Hz, 1H), 5.41 (dd, J=6.00, 13.00 Hz, 2H), 4.39-4.24 (m, 2H), 3.84-3.65 (m, 4H), 3.18-2.91 (m, 4H), 2.76-2.71 (m, 1H), 2.11-2.06 (m, 4H), 1.90-1.76 (m, 2H), 1.48-1.39 (m, 8H), 1.24-1.13 (m, 1H). [4 protons are merged in the solvent peak]. LCMS (ES+): m/z 1012.0 [M+H]⁺.

### Example 164

### 3-(carboxymethoxy)-4-chloro-5-(3-(((4S)-1-((3-(4-(1-(2,6-dioxopiperidin-3-yl)-2-oxo-1,2-dihydrobenzo[cd]indol-5-yl)-1,4-diazepane-1-carboxamido)benzyl)sulfonyl)-2,2-dimethylpiperidin-4-yl)amino)phenyl)thiophene-2-carboxylic acid (164)

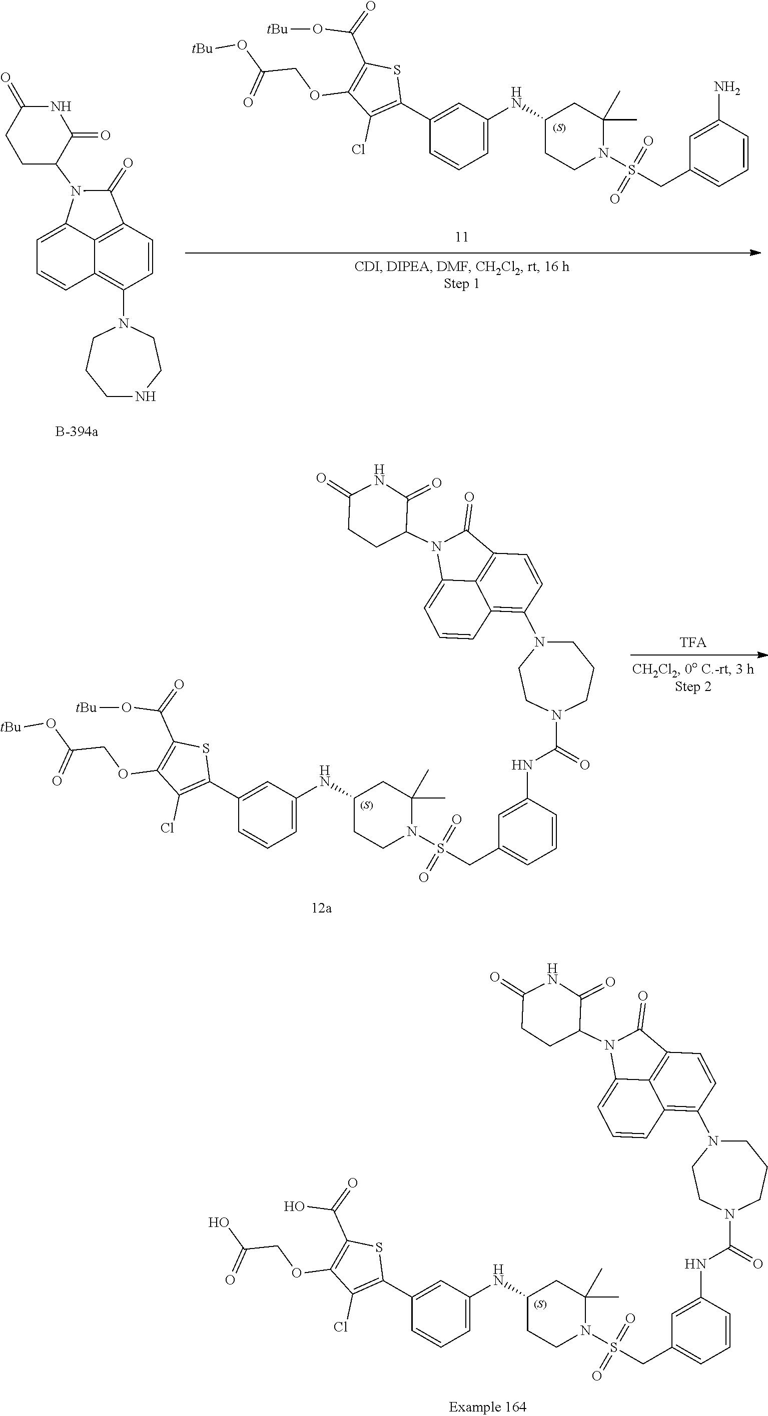

### Step 1: tert-butyl 3-(2-(tert-butoxy)-2-oxoethoxy)-4-chloro-5-(3-(((4S)-1-((3-(4-(1-(2,6-dioxopiperidin-3-yl)-2-oxo-1,2-dihydrobenzo[cd]indol-5-yl)-1,4-diazepane-1-carboxamido)benzyl)sulfonyl)-2,2-dimethylpiperidin-4-yl)amino)phenyl)thiophene-2-carboxylate (12a)

tert-butyl 3-(2-tert-butoxy-2-oxo-ethoxy)-4-chloro-5-[3-[[(4S)-1-[3-[4-[1-(2,6-dioxo-3-piperidyl)-2-oxo-benzo[cd]indol-5-yl]-1,4-diazepane-1-carbonyl]amino]phenyl]methylsulfonyl]-2,2-dimethyl-4-piperidyl]amino]phenyl]thiophene-2-carboxylate (12a, 200 mg, 77.88 μmol, 45% yield, 44% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (11, 188.39 mg, 258.91 μmol) and 3-[5-(1,4-diazepan-1-yl)-2-oxo-benzo[cd]indol-1-yl]piperidine-2,6-dione (B-394a, 100 mg, 172.61 μmol, TFA salt) in a similar fashion to Compound B-242, and was used in the next reaction without further purification.

LCMS (ES+): m/z 1124.2 [M+H]⁺

### Step 2: 3-(carboxymethoxy)-4-chloro-5-(3-(((4S)-1-((3-(4-(1-(2,6-dioxopiperidin-3-yl)-2-oxo-1,2-dihydrobenzo[cd]indol-5-yl)-1,4-diazepane-1-carboxamido)benzyl)sulfonyl)-2,2-dimethylpiperidin-4-yl)amino)phenyl)thiophene-2-carboxylic acid (164)

3-(carboxymethoxy)-4-chloro-5-[3-[[(4S)-1-[[3-[[4-[1-(2,6-dioxo-3-piperidyl)-2-oxo-benzo[cd]indol-5-yl]-1,4-diazepane-1-carbonyl]amino]phenyl]methylsulfonyl]-2,2-dimethyl-4-piperidyl]amino]phenyl]thiophene-2-carboxylic acid (164, 24 mg, 22.13 μmol, 28% yield, formic acid salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-5-yl]-1,4-diazepane-1-carbonyl]amino]phenyl]methylsulfonyl]-2,2-dimethyl-4-piperidyl]amino]phenyl]thiophene-2-carboxylate (12a, 200 mg, 77.88 μmol) in a similar fashion to Compound A-26. The residue was then purified by reverse-phase preparative HPLC [Column: X-BRIDGE C8 (150×19) mm, 5 μm; Mobile phase A: 0.1% TFA in water and Mobile Phase B: MeCN]. ¹H-NMR (400 MHz, DMSO-d6): δ 11.09 (s, 1H), 8.47 (d, J=Hz, 1H), 7.87 (d, J=8.00 Hz, 1H), 7.73 (d, J=8.80 Hz, 1H), 7.52 (s, 1H), 7.43-7.35 (m, 2H), 7.24-7.17 (m, 2H), 7.11 (d, J=8.00 Hz, 1H), 7.06 (d, J=6.80 Hz, 1H), 6.98 (d, J=7.20 Hz, 1H), 6.86 (s, 1H), 6.80 (d, J=7.20 Hz, 1H), 6.68 (d, J=8.00 Hz, 1H), 5.66 (d, J=4.40 Hz, 1H), 5.39 (d, J=10.00 Hz, 2H), 4.90 (s, 2H), 4.35-4.20 (m, 4H), 3.88-3.70 (m, 7H), 3.10-2.80 (m, 2H), 2.12-2.04 (m, 5H), 1.83-1.73 (m, 2H), 1.44 (s, 3H), 1.37 (s, 3H), 1.08-1.06 (m, 1H). LCMS (ES+): m/z 1012.0 [M+H]⁺.

### Example 165

### 3-(carboxymethoxy)-4-chloro-5-(3-(((4S)-1-((3-((1r,4S)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylic acid (165)

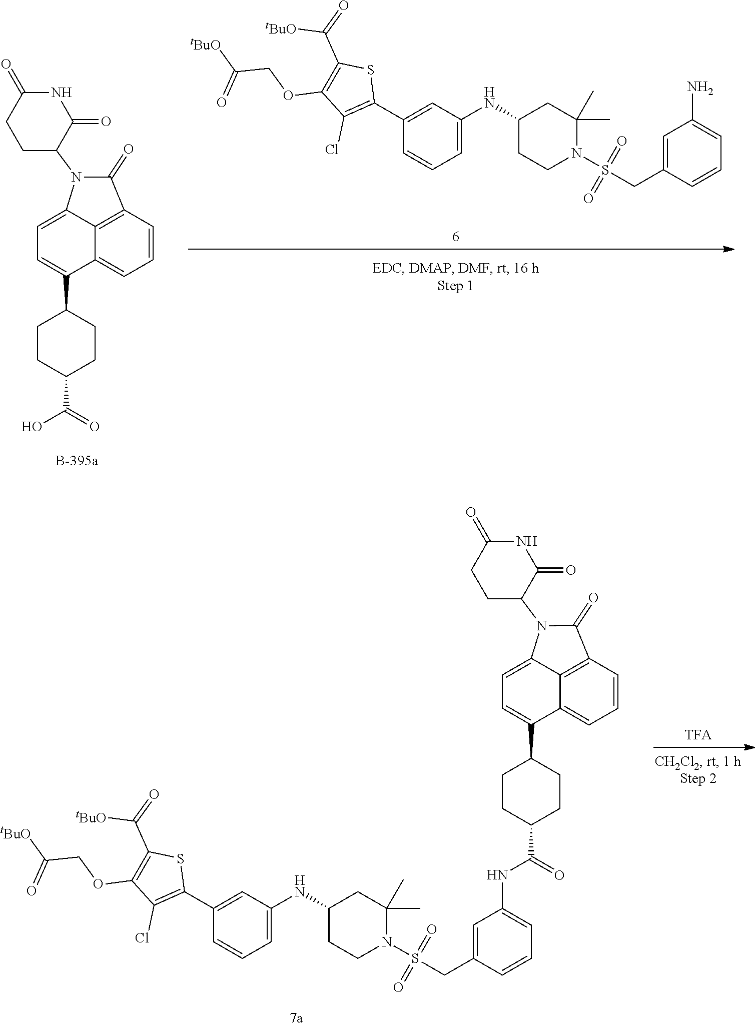 [SMILES: O=C1CCC(N2C(=O)c3cccc4c3c2ccc4[C@H]2CC[C@H](C(=O)O)CC2)C(=O)N1 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)[C@H]5CC[C@H](c6ccc7c8c(cccc86)C(=O)N7C6CCC(=O)NC6=O)CC5)c4)C(C)(C)C3)c2)c1Cl]

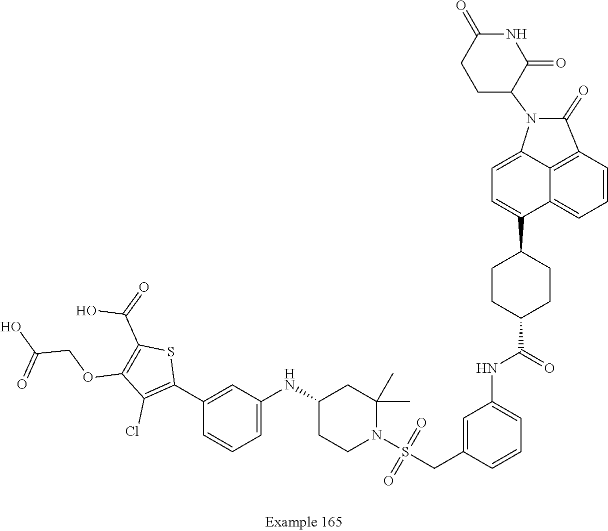 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@H](c3ccc4c5c(cccc53)C(=O)N4C3CCC(=O)NC3=O)CC2)c1]

### Step 1: tert-butyl 3-(2-(tert-butoxy)-2-oxoethoxy)-4-chloro-5-(3-(((4S)-1-((3-((1r,4S)-4-(1-(2,6-dioxopiperidin-3-yl)-2-oxo-1,2-dihydrobenzo[cd]indol-6-yl))cyclohexane-1-carboxamido)benzyl)sulfonyl)-2,2-dimethylpiperidin-4-yl)amino)phenyl)thiophene-2-carboxylate (7a, From the First Eluted Fraction)

Into a 25 mL single neck round bottom flask containing a well-stirred solution of (1r,4r)-4-(1-(2,6-dioxopiperidin-3-yl)-2-oxo-1,2-dihydrobenzo[cd]indol-6-yl)cyclohexane-1-carboxylic acid (B-395a, 50 mg, 106.54 μmol) in dry DMF (2 mL) was added EDC·HCl (61.11 mg, 319.61 μmol) followed by DMAP (65.08 mg, 532.69 μmol) and the resulting mixture was stirred for 30 min. Subsequently, tert-butyl(S)-5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (6, 78.07 mg, 106.54 μmol) was added. After 16 h, the solvent was evaporated under reduced pressure and the residue was diluted with ice-cold water to precipitate a solid that was filtered and dried to afford tert-butyl 3-(2-(tert-butoxy)-2-oxoethoxy)-4-chloro-5-(3-(((4S)-1-((3-((1r,4S)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxy late (7a, 100 mg, 37.88 μmol, 36% yield, 42% purity) as an yellow solid. LCMS (ES−): m/z 1106.6 [M−H]⁻

### Step 2: 3-(carboxymethoxy)-4-chloro-5-(3-(((4S)-1-((3-((1r,4S)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylic acid (165)

3-(carboxymethoxy)-4-chloro-5-(3-(((4S)-1-((3-((1r,4S)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylic acid (165, 11 mg, 9.73 μmol, 26% yield, TFA salt) was synthesized from tert-butyl 3-(2-(tert-butoxy)-2-oxoethoxy)-4-chloro-5-(3-(((4S)-1-((3-((1r,4S)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylate (7a, 100 mg, 37.88 μmol) in a similar fashion to Compound A-26. The residue was purified by reverse-phase preparative HPLC [Column: SUNFIRE C18 5 MICRON 19×150 mm; Mobile phase A: 0.1% TFA in water and Mobile phase B: MeCN]. ¹H-NMR (400 MHz, DMSO-d6): δ 11.13 (s, 1H), 10.00 (s, 1H), 8.44 (d, J=8.40 Hz, 1H), 8.11 (d, J=6.80 Hz, 1H), 7.86 (t, J=7.60 Hz, 1H), 7.76 (s, 1H), 7.63 (d, J=7.60 Hz, 1H), 7.38 (d, J=7.20 Hz, 1H), 7.32 (t, J=8.00 Hz, 1H), 7.20 (t, J=8.00 Hz, 1H), 7.11-7.08 (m, 2H), 6.89 (s, 1H), 6.81 (d, J=7.20 Hz, 1H), 6.71 (d, J=8.00 Hz, 1H), 5.68 (s, 1H), 5.45 (dd, J=5.20, 12.80 Hz, 1H), 4.87 (s, 2H), 4.43-4.29 (m, 2H), 3.60-3.48 (m, 4H), 3.18-3.14 (m, 2H), 3.01-2.85 (m, 1H), 2.72-2.66 (m, 1H), 2.13-2.01 (m, 5H), 1.90-1.82 (m, 4H), 1.65-1.62 (m, 2H), 1.49-1.41 (m, 7H), 1.24-1.15 (m, 1H). LCMS (ES+): m/z 995.7 [M+H]⁺.

### Example 166

### 3-(carboxymethoxy)-4-chloro-5-(3-(((4S)-1-((3-((1s,4R)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylic acid (166)

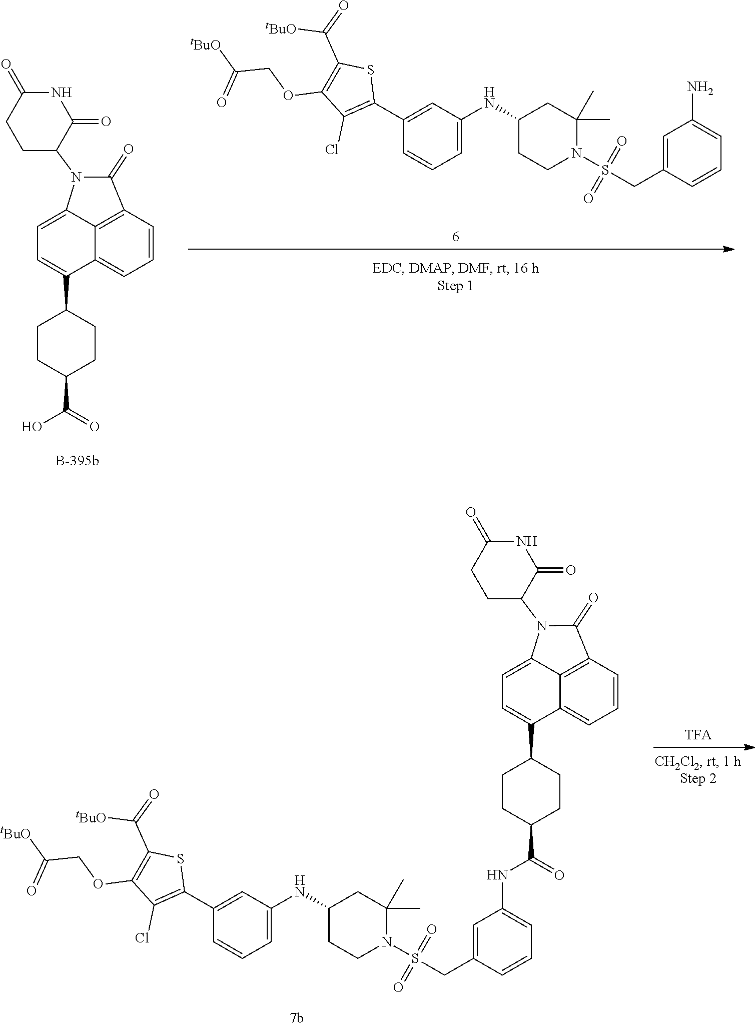 [SMILES: O=C1CCC(N2C(=O)c3cccc4c3c2ccc4[C@H]2CC[C@@H](C(=O)O)CC2)C(=O)N1 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)[C@H]5CC[C@@H](c6ccc7c8c(cccc86)C(=O)N7C6CCC(=O)NC6=O)CC5)c4)C(C)(C)C3)c2)c1Cl]

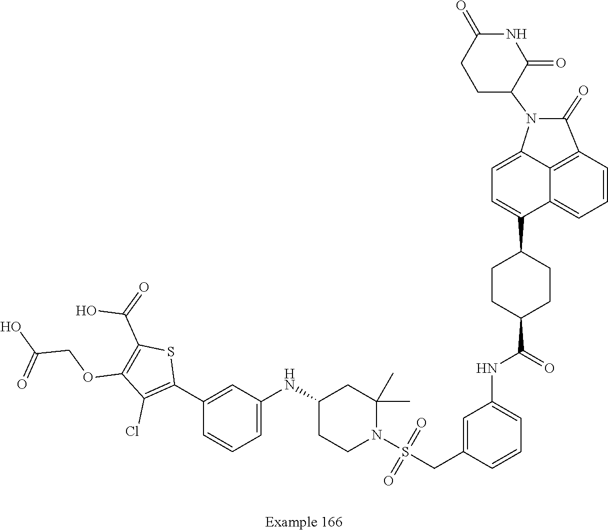 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@@H](c3ccc4c5c(cccc53)C(=O)N4C3CCC(=O)NC3=O)CC2)c1]

### Step 1: tert-butyl 3-(2-tert-butoxy)-2-oxoethoxy)-4-chloro-5-(3-(((4S)-1-((3-((1s,4R)-4-(1-(2,6 dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylate (7b)

Into a 25 mL single neck round bottom flask containing a well-stirred solution of (1s,4s)-4-(1-(2,6-dioxopiperidin-3-yl)-2-oxo-1,2-dihydrobenzo[cd]indol-6-yl)cyclohexane-1-carboxylic acid (B-395b, 70 mg, 128.48 μmol) in dry DMF (1.5 mL) was added EDC·HCl (73.89 mg, 385.45 μmol) followed by DMAP (78.48 mg, 642.42 μmol) at ambient temperature and the resulting mixture was stirred for 30 min. Tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (6, 94.15 mg, 128.48 μmol) was added and stirring was continued for another 16 h. The solvent was evaporated and the residue diluted with ice-cold water to precipitate a solid. Filtration and drying over vacuum afforded tert-butyl 3-(2-(tert-butoxy)-2-oxoethoxy)-4-chloro-5-(3-(((4S)-1-((3-((1s,4R)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylate (7b, 130 mg, 69.18 μmol, 54% yield, 59% purity) as a yellow solid. LCMS (ES+): m/z 1108.2 [M+H]⁺

### Step 2: 3-(carboxymethoxy)-4-chloro-5-(3-(((4S)-1-((3-((1s,4R)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylic acid (166)

3-(carboxymethoxy)-4-chloro-5-(3-(((4S)-1-((3-((1s,4R)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxylic acid (166, 25 mg, 21.86 μmol, 32% yield, TFA salt) was synthesized from tert-butyl 3-(2-(tert-butoxy)-2-oxoethoxy)-4-chloro-5-(3-(((4S)-1-((3-((1s,4R)-4-(1-(2,6-dioxopiperidin-3-yl)-2-oxo-1,2-dihydrobenzo[cd]indol-6-yl)cyclohexane-1-carboxamido)benzyl)sulfonyl)-2,2-dimethylpiperidin-4-yl)amino)phenyl)thiophene-2-carboxy late (7b, 130 mg, 69.18 μmol) in a similar fashion to Compound A-26. The residue was purified by reverse-phase preparative HPLC [Column: SUNFIRE C18, 5 MICRON 19×150 mm; Mobile phase A: 0.1% TFA in water and Mobile Phase B: MeCN]. H-NMR (400 MHz, DMSO-d6): δ 11.12 (s, 1H), 9.91 (s, 1H), 8.44 (d, J=8.00 Hz, 1H), 8.09 (d, J=6.80 Hz, 1H), 7.86 (t, J=7.20 Hz, 1H), 7.74 (s, 1H), 7.66 (d, J=8.00 Hz, 1H), 7.32-6.88 (m, 5H), 6.90 (s, 1H), 6.78 (d, J=7.20 Hz, 1H), 6.66 (d, J=8.00 Hz, 1H), 5.67 (d, J=6.00 Hz, 1H), 5.43 (d, J=12.00 Hz, 1H), 4.85 (s, 2H), 4.43-4.25 (m, 2H), 3.14-2.71 (m, 3H), 2.16-2.08 (m, 6H), 1.87-1.66 (m, 8H), 1.52-1.30 (m, 9H), 1.24-1.13 (m, 1H). LCMS (ES+): m/z 995.7 [M+H]⁺.

### Example 167

### 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-4-fluorobenzyl)sulfonyl)-2,2-dimethylpiperidin-4-yl)amino)-2-fluorophenyl)thiophene-2-carboxylic acid (167)

Configuration is arbitrarily assigned.

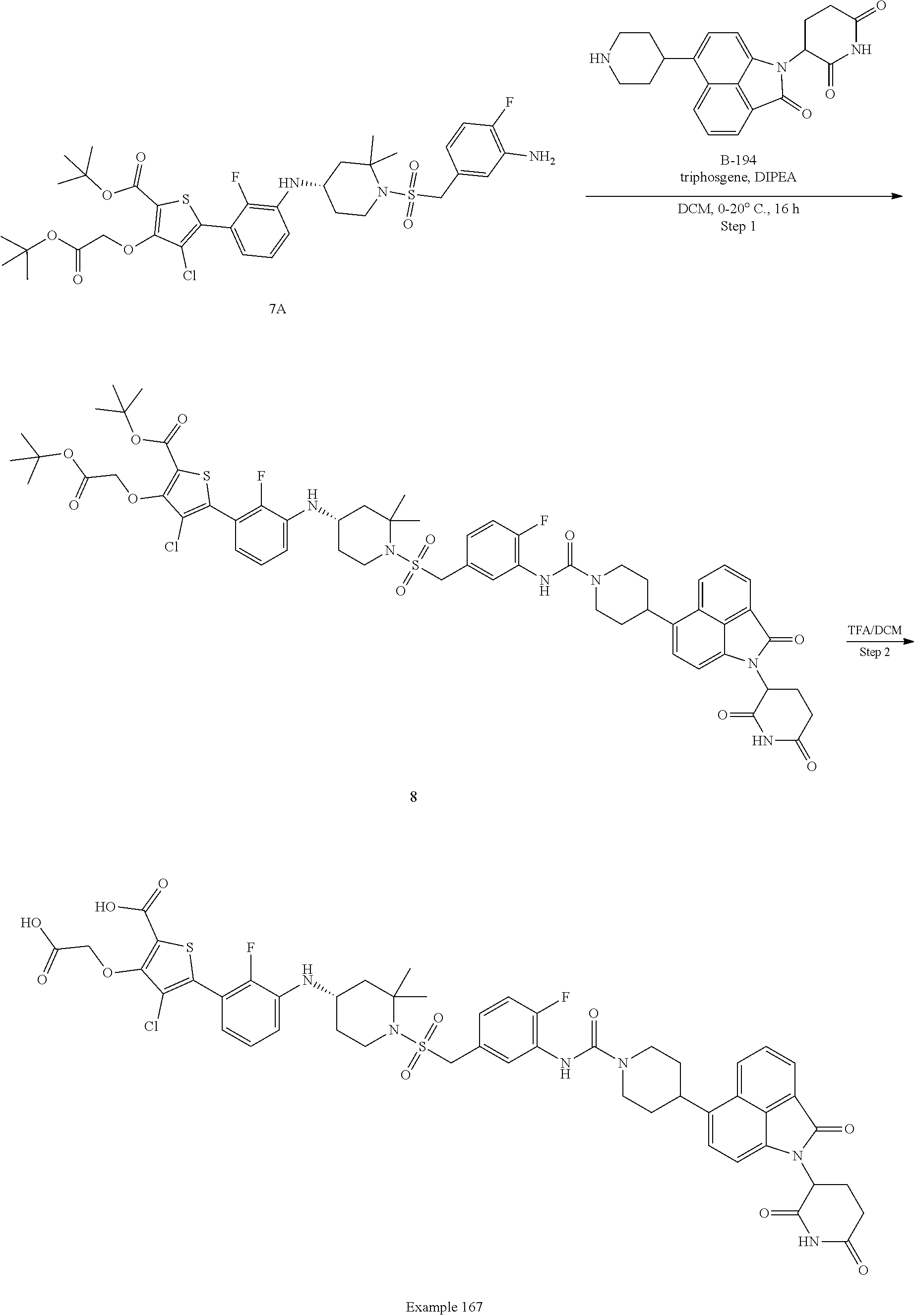 [SMILES: CC(C)(C)OC(=O)COc1c(C(=O)OC(C)(C)C)sc(-c2cccc(N[C@H]3CCN(S(=O)(=O)Cc4ccc(F)c(N)c4)C(C)(C)C3)c2F)c1Cl ; O=C1CCC(N2C(=O)c3cccc4c(C5CCNCC5)ccc2c34)C(=O)N1 ; CC(C)(C)OC(=O)COc1c(C(=O)OC(C)(C)C)sc(-c2cccc(N[C@H]3CCN(S(=O)(=O)Cc4ccc(F)c(NC(=O)N5CCC(c6ccc7c8c(cccc68)C(=O)N7C6CCC(=O)NC6=O)CC5)c4)C(C)(C)C3)c2F)c1Cl ; CC1(C)C[C@@H](Nc2cccc(-c3sc(C(=O)O)c(OCC(=O)O)c3Cl)c2F)CCN1S(=O)(=O)Cc1ccc(F)c(NC(=O)N2CCC(c3ccc4c5c(cccc35)C(=O)N4C3CCC(=O)NC3=O)CC2)c1]

### Step 1: tert-butyl 3-(2-(tert-butoxy)-2-oxoethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-4-fluorobenzyl)sulfonyl)-2,2-dimethylpiperidin-4-yl)amino)-2-fluorophenyl)thiophene-2-carboxylate (8)

To a mixture of(S)-tert-butyl 5-(3-((1-((3-amino-4-fluorobenzyl)sulfonyl)-2,2-dimethylpiperidin-4-yl)amino)-2-fluorophenyl)-3-(2-(tert-butoxy)-2-oxoethoxy)-4-chlorothiophene-2-carboxylate (7A, 120 mg, 158.66 μmol, Peak1) and DIPEA (205.06 mg, 1.59 mmol, 276.36 μL) in DCM (5 mL) was added triphosgene (47.08 mg, 158.66 μmol) at 0° C., the mixture was stirred at 20° C. for 15 min. Then 3-(2-oxo-6-(piperidin-4-yl)benzo[cd]indol-[(2H)-yl)piperidine-2,6-dione (B-194, 57.66 mg, 158.66 μmol) was added to the mixture. The mixture was stirred at 20° C. for 16 h. The mixture was poured into water (30 mL). The mixture was extracted with EtOAc (10 mL). The organic phase was washed with brine (30 mL), dried with anhydrous Na₂SO₄, filtered and concentrated in vacuum. The residue was purified by prep-TLC (Petroleum ether/Ethyl acetate=1/2, RF=0.4) to afford tert-butyl 3-(2-(tert-butoxy)-2-oxoethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-4-fluorobenzyl)sulfonyl)-2,2-dimethylpiperidin-4-yl)amino)-2-fluorophenyl)thiophene-2-carboxylate (8, 64 mg, 54.74 μmol, 35% yield) as yellow solid. LCMS (ESI): m/z 1145.5 [M+H]⁺

### Step 2: 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-4-fluorobenzyl)sulfonyl)-2,2-dimethylpiperidin-4-yl)amino)-2-fluorophenyl)thiophene-2-carboxylic acid (167)

3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-4-fluorobenzyl)sulfonyl)-2,2-dimethylpiperidin-4-yl)amino)-2-fluorophenyl)thiophene-2-carboxylic acid (167, 45.89 mg, 39.99 μmol, 57% yield, TFA salt) was synthesized from tert-butyl 3-(2-(tert-butoxy)-2-oxoethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)-4-fluorobenzyl)sulfonyl)-2,2-dimethylpiperidin-4-yl)amino)-2-fluorophenyl)thiophene-2-carboxylate (8, 80 mg, 69.83 μmol) in a similar fashion to Compound A-26. The residue was purified by prep-HPLC (flow: 25 mL/min; gradient: from 19-49% MeCN in water (0.1% TFA); column: Phenomenex Synergi C18 150×25 mm×10 μm). LCMS (ESI): m/z 1034.0 [M+H]⁺

¹H NMR (400 MHz, DMSO-d₆) δ 11.12 (s, 1H), 8.48 (d, J=8.4 Hz, 1H), 8.40 (s, 1H), 8.11 (d, J=7.0 Hz, 1H), 7.87 (dd, J=7.0, 8.2 Hz, 1H), 7.55 (dd, J=1.8, 7.6 Hz, 1H), 7.35 (d, J=7.6 Hz, 1H), 7.25-7.13 (m, 2H), 7.11-7.04 (m, 2H), 6.98-6.90 (m, 1H), 6.67-6.58 (m, 1H), 5.43 (br dd, J=4.4, 12.8 Hz, 1H), 5.38-5.13 (m, 1H), 4.89 (d, J=0.8 Hz, 2H), 4.43 (br d, J=13.8 Hz, 1H), 4.36-4.27 (m, 3H), 3.57 (br s, 2H), 3.10 (q, J=11.6 Hz, 3H), 3.00-2.91 (m, 1H), 2.82-2.72 (m, 1H), 2.69-2.63 (m, 1H), 2.52 (br d, J=1.8 Hz, 1H), 2.09 (br dd, J=4.6, 10.4 Hz, 1H), 1.90 (br d, J=11.4 Hz, 2H), 1.83-1.69 (m, 4H), 1.66-1.58 (m, 1H), 1.49 (s, 3H), 1.39 (s, 3H), 1.25-1.15 (m, 1H).

### Example 168

### 3-(carboxymethoxy)-4-chloro-5-(3-(((4S)-1-((3-((4R)-4-(1-(2,6-dioxopiperidin-3-yl)-3-methyl-2-oxo-2,3-dihydro-1H-benzo[d]imidazol-5-yl)-3,3-difluoropiperidine-1-carboxamido)benzyl)sulfonyl)-2,2-dimethylpiperidin-4-yl)amino)phenyl)thiophene-2-carboxylic acid (168)

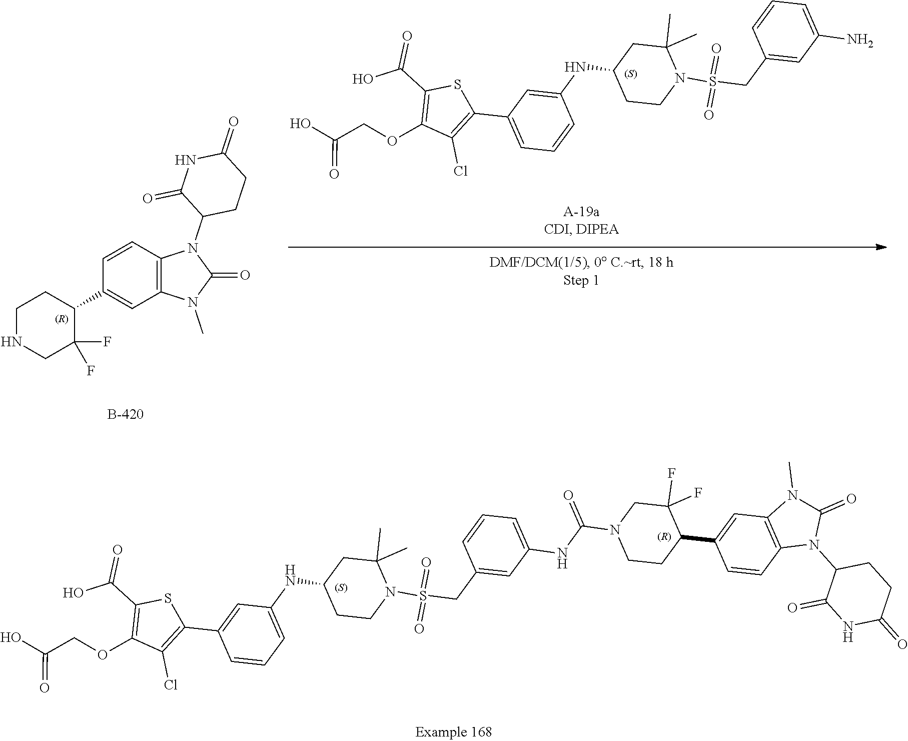

### Step 1: 3-(carboxymethoxy)-4-chloro-5-(3-(((4S)-1-((3-((4R)-4-(1-(2,6-dioxopiperidin-3-yl)-3-methyl-2-oxo-2,3-dihydro-1H-benzo[d]imidazol-5-yl)-3,3-difluoropiperidine-1-carboxamido)benzyl)sulfonyl)-2,2-dimethylpiperidin-4-yl)amino)phenyl)thiophene-2-carboxylic acid (168)

3-(carboxymethoxy)-4-chloro-5-(3-(((4S)-1-((3-((4R)-4-(1-(2,6-dioxopiperidin-3-yl)-3-methyl-2-oxo-2,3-dihydro-1H-benzo[d]imidazol-5-yl)-3,3-difluoropiperidine-1-carboxamido)benzyl)sulfonyl)-2,2-dimethylpiperidin-4-yl)amino)phenyl)thiophene-2-carboxylic acid (168, 127.7 mg, 113.36 μmol, 27% yield, TFA salt) was synthesized from(S)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19a, 257.15 mg, 422.86 μmol) and 3-(5-((R)-3,3-difluoropiperidin-4-yl)-3-methyl-2-oxo-2,3-dihydro-1H-benzo[d]imidazol-1-yl)piperidine-2,6-dione (B-420, 160 mg, 422.86 μmol, HCl salt) in a similar fashion to Compound B-242. The residue was purified by prep-HPLC (flow: 25 mL/min; gradient: from 38-68% MeCN-water (0.1% TFA) over 7 min; column: 3_Phenomenex Luna C18 75×30 mm×3 μm). LCMS (ESI): m/z 1012.9 [M+H]⁺

¹H NMR (400 MHz, DMSO-d₆) δ 11.10 (s, 1H), 8.79 (s, 1H), 7.58 (s, 1H), 7.49 (d, J=8.2 Hz, 1H), 7.27 (t, J=7.6 Hz, 1H), 7.19 (t, J=7.6 Hz, 1H), 7.14 (s, 1H), 7.09-7.05 (m, 1H), 7.00 (dd, J=7.6, 12.8 Hz, 2H), 6.87 (s, 1H), 6.81 (d, J=7.6 Hz, 1H), 6.69 (dd, J=1.2, 8.0 Hz, 1H), 5.36 (dd, J=5.2, 12.8 Hz, 1H), 4.91 (s, 2H), 4.64-4.54 (m, 1H), 4.41-4.32 (m, 2H), 4.27 (d, J=13.2 Hz, 1H), 3.34 (s, 3H), 3.29 (s, 1H), 3.18-3.06 (m, 3H), 3.06 (s, 1H), 2.89 (dd, J=5.2, 16.0 Hz, 1H), 2.72 (d, J=4.0 Hz, 1H), 2.69-2.56 (m, 2H), 2.22-2.13 (m, 1H), 2.05-1.98 (m, 1H), 1.92-1.83 (m, 2H), 1.79 (d, J=11.2 Hz, 1H), 1.52-1.37 (m, 7H), 1.15-1.06 (m, 1H).

### Example 169

### 3-(carboxymethoxy)-4-chloro-5-(3-(((4S)-1-((3-((4S)-4-(1-(2,6-dioxopiperidin-3-yl)-3-methyl-2-oxo-2,3-dihydro-1H-benzo[d]imidazol-5-yl)-3,3-difluoropiperidine-1-carboxamido)benzyl)sulfonyl)-2,2-dimethylpiperidin-4-yl)amino)phenyl)thiophene-2-carboxylic acid (169)

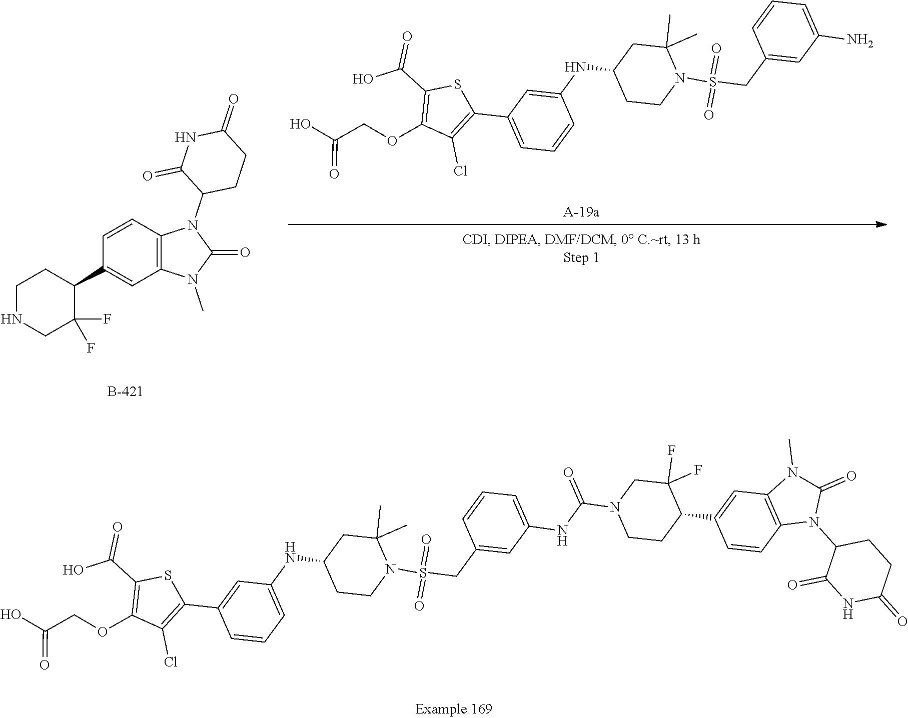 [SMILES: Cn1c(=O)n(C2CCC(=O)NC2=O)c2ccc([C@@H]3CCNCC3(F)F)cc21 ; CC1(C)C[C@@H](Nc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc([C@@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21]

### Step 1: 3-(carboxymethoxy)-4-chloro-5-(3-(((4S)-1-((3-((4.5)-4-(1-(2,6-dioxopiperidin-3-yl)-3-methyl-2-oxo-2,3-dihydro-1H-benzo[d]imidazol-S-yl)-3,3-difluoropiperidine-1-carboxamido)benzyl)sulfonyl)-2,2-dimethylpiperidin-4-yl)amino)phenyl)thiophene-2-carboxylic acid (169)

3-(carboxymethoxy)-4-chloro-5-(3-(((4S)-1-((3-((4S)-4-(1-(2,6-dioxopiperidin-3-yl)-3-methyl-2-oxo-2,3-dihydro-1H-benzo[d]imidazol-5-yl)-3,3-difluoropiperidine-1-carboxamido)benzyl)sulfonyl)-2,2-dimethylpiperidin-4-yl)amino)phenyl)thiophene-2-carboxylic acid (169, 69.1 mg, 61.34 μmol, 27% yield, TFA salt) was synthesized from(S)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19a, 140 mg, 230.22 μmol) and 3-(5-((S)-3,3-difluoropiperidin-4-yl)-3-methyl-2-oxo-2,3-dihydro-1H-benzo[d]imidazol-1-yl)piperidine-2,6-dione (B-421, 95.50 mg, 230.22 μmol, HCl salt) in a similar fashion to Compound B-242. The residue was purified by prep-HPLC (flow: 25 mL/min; gradient: from 38-68% MeCN in water (0.1% TFA) over 9 min; column: 3_Phenomenex luna C18 75×30 mm×3 μm). LCMS (ESI): m/z 1012.6 [M+H]⁺

¹H NMR (400 MHz, DMSO-d₆) δ 11.10 (s, 1H), 8.79 (s, 1H), 7.57 (s, 1H), 7.49 (d, J=8.8 Hz, 1H), 7.27 (t, J=8.0 Hz, 1H), 7.20 (t, J=8.0 Hz, 1H), 7.14 (s, 1H), 7.10-7.05 (m, 1H), 7.03-6.97 (m, 2H), 6.88 (s, 1H), 6.81 (d, J=7.6 Hz, 1H), 6.70 (d, J=8.0 Hz, 1H), 5.39-5.31 (m, 1H), 4.91 (s, 2H), 4.66-4.52 (m, 1H), 4.42-4.23 (m, 3H), 3.47-3.22 (m, 6H), 3.18-2.99 (m, 2H), 2.97-2.83 (m, 1H), 2.79-2.57 (m, 2H), 2.25-2.09 (m, 1H), 2.07-1.95 (m, 1H), 1.93-1.74 (m, 3H), 1.48 (s, 3H), 1.40 (s, 4H), 1.19-1.01 (m, 1H).

### Example 169a and Example 169b

### 3-(carboxymethoxy)-4-chloro-5-[3-[[(4S)-2,2-dimethyl-1-[[3-[(4S)-3,3-difluoro-4-[3-methyl-2-oxo-1-[(35)-2,6-dioxo-3-piperidyl]benzimidazol-5-yl]piperidine-1-carbonyl]amino]phenyl]methylsulfonyl]-4-piperidyl]amino]phenyl]thiophene-2-carboxylic acid (169a) and 3-(carboxymethoxy)-4-chloro-5-[3-[[(4S)-2,2-dimethyl-1-[[3-[[(4S)-3,3-difluoro-4-[3-methyl-2-oxo-1-[(3R)-2,6-dioxo-3-piperidyl]benzimidazol-5-yl]piperidine-1-carbonyl]amino]phenyl]methylsulfonyl]-4-piperidyl]amino]phenyl]thiophene-2-carboxylic acid (169b)

Configurations are arbitrarily assigned.

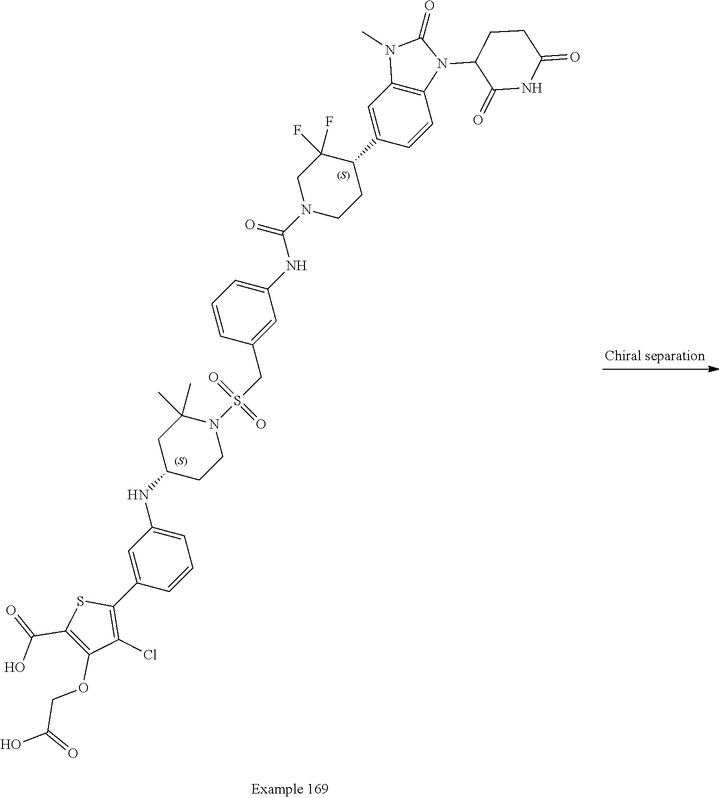

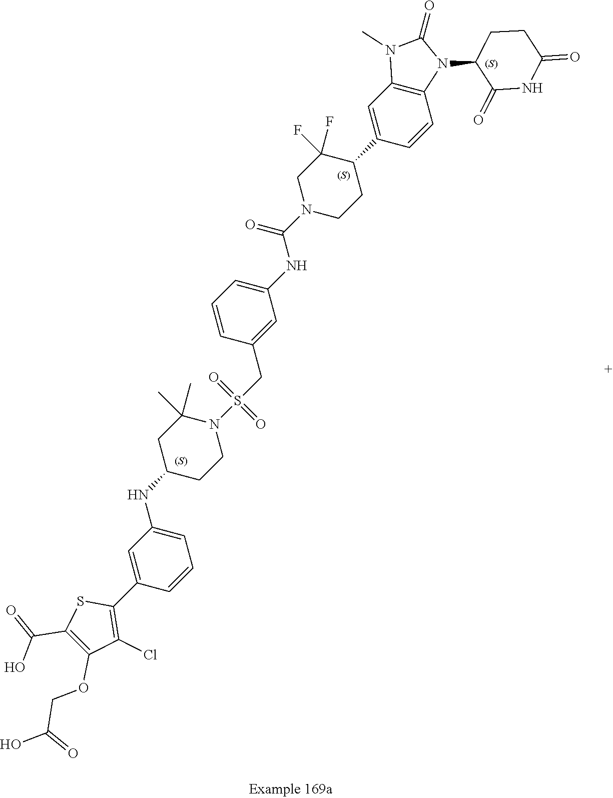

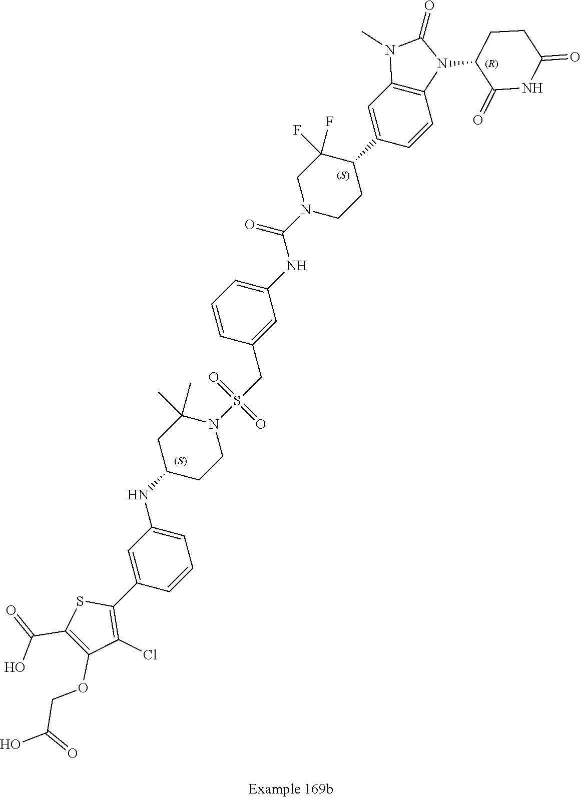

3-(carboxymethoxy)-4-chloro-5-[3-[[(4S)-2,2-dimethyl-1-[[3-[[(4S)-4-[1-(2,6-dioxo-3-piperidyl)-3-methyl-2-oxo-benzimidazol-5-yl]-3,3-difluoro-piperidine-1-carbonyl]amino]phenyl]methylsulfonyl]-4-piperidyl]amino]phenyl]thiophene-2-carboxylic acid (169, 50 mg, 44.38 μmol, TFA salt) was subjected to chiral SFC purification to separate the diastereomers. Method details: Column Name: YMC Cellulose-SC, Flowrate: 5 ml/min, Co-Solvent: 50%, Co-Solvent Name: 0.2% TFA in IPA:ACN (1:1), Injected Volume: 15 μL, Temperature: 35° C., Outlet Pressure: 100 bar.

### Chiral SFC:

### Method of Analysis

Instrument: PIC 175
Column: YMC Cellulose SC (250×30) mm, 5 μm
Mobile Phase: CO₂: 0.2% Trifluoracetic acid in Isopropyl alcohol:Acetonitrile (1:1):(50:50)
Total Flow: 90 g/min
Back pressure: 120 bar
Wave length: 210 nm
Cycle time: 25 min

50 mg of Compound 169 was dissolved in 2 mL of ACN and injected 900 μL/injection.

After SFC purification, the first eluted fraction at rt 4.8 min was concentrated under reduced pressure at 40° C. and purified by reverse-phase column chromatography [Silicycle C18 column, mobile phase:

0.1% TFA in water and MeCN] to afford 3-(carboxymethoxy)-4-chloro-5-[3-[[(4S)-2,2-dimethyl-1-[[3-[[(4S)-3,3-difluoro-4-[3-methyl-2-oxo-1-[(3S)-2,6-dioxo-3-piperidyl]benzimidazol-5-yl]piperidine-1-carbonyl]amino]phenyl]methylsulfonyl]-4-piperidyl]amino]phenyl]thiophene-2-carboxylic acid (169a, 14 mg, 12.34 μmol, 28% yield, TFA salt) as an off-white solid.

### Fraction 1: 169a:

¹H-NMR (400 MHz, DMSO-d6): δ 11.10 (s, 1H), 8.81 (s, 1H), 7.58 (s, 1H), 7.50 (s, 1H), 7.28 (t, J=8.00 Hz, 1H), 7.20 (t, J=8.00 Hz, 1H), 7.15 (s, 1H), 7.08 (d, J=8.40 Hz, 1H), 7.04-6.99 (m, 2H), 6.87 (s, 1H), 6.80 (d, J=8.00 Hz, 1H), 6.70-6.68 (m, 1H), 5.69 (s, 1H), 5.41-5.31 (m, 1H), 4.90 (s, 2H), 4.65-4.51 (m, 2H), 4.40-4.26 (m, 3H), 3.15-3.01 (m, 6H), 2.97-2.85 (m, 2H), 2.23-2.12 (m, 2H), 2.05-1.95 (m, 2H), 1.91-1.73 (m, 4H), 1.41 (s, 3H), 1.49 (s, 3H), 1.20-1.09 (m, 2H). LCMS (ES+): m/z 1012.1 [M+H]⁺

Similarly, the second eluted fraction at rt 12.1 min was concentrated under reduced pressure at 40° C. and purified by reverse-phase column chromatography [Silicycle C18 column, mobile phase: 0.1% TFA in water and MeCN] to afford 3-(carboxymethoxy)-4-chloro-5-[3-[[(4S)-2,2-dimethyl-1-[[3-[(4S)-3,3-difluoro-4-[3-methyl-2-oxo-1-[(3R)-2,6-dioxo-3-piperidyl]benzimidazol-5-yl]piperidine-1-carbonyl]amino]phenyl]methylsulfonyl]-4-piperidyl]amino]phenyl]thiophene-2-carboxylic acid (169b, 17 mg, 15.09 μmol, 34% yield, TFA salt) as an off-white solid.

### Fraction 2: 169b:

¹H-NMR (400 MHz, DMSO-d6): δ 11.10 (s, 1H), 8.82 (s, 1H), 7.58 (s, 1H), 7.51-7.49 (m, 1H), 7.27 (t, J=8.00 Hz, 1H), 7.20 (t, J=8.00 Hz, 1H), 7.15 (s, 1H), 7.08 (d, J=8.00 Hz, 1H), 7.03-6.98 (m, 2H), 6.87 (s, 1H), 6.80 (d, J=7.60 Hz, 1H), 6.69 (dd, J=1.60, 8.20 Hz, 1H), 5.70 (s, 1H), 5.37-5.31 (m, 1H), 4.90 (s, 2H), 4.55-4.51 (m, 2H), 4.40-4.25 (m, 3H), 3.15-3.01 (m, 6H), 2.99-2.85 (m, 2H), 2.23-2.11 (m, 2H), 2.05-1.95 (m, 2H), 1.91-1.73 (m, 4H), 1.52 (s, 3H), 1.40 (s, 3H), 1.20-1.09 (m, 2H), LCMS (ES+): m/z 1012.1 [M+H]⁺.

### Example 170

### 3-(carboxymethoxy)-4-chloro-5-[3-[[(4S)-1-[[3-[[4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (170)

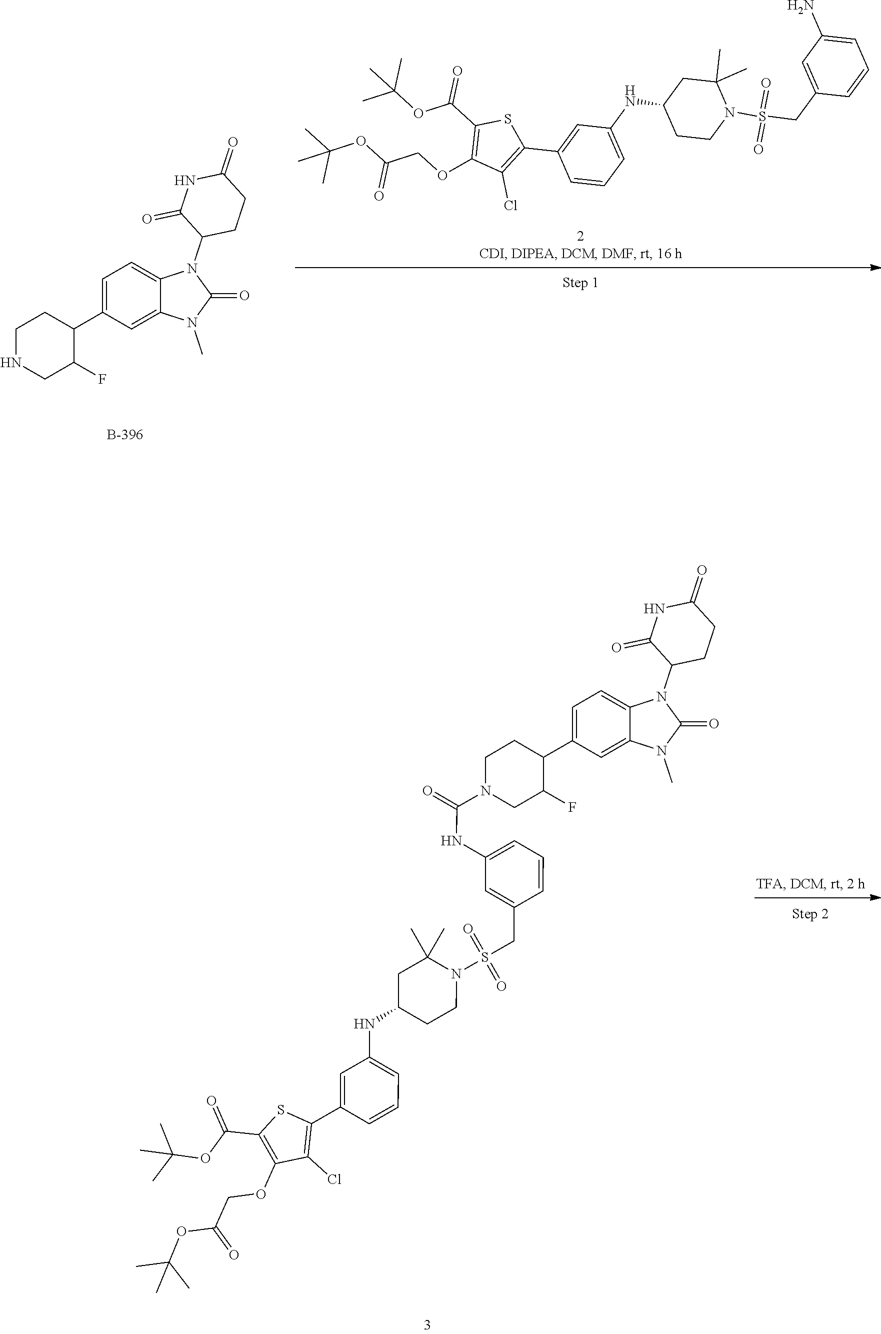 [SMILES: Cn1c(=O)n(C2CCC(=O)NC2=O)c2ccc(C3CCNCC3F)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(C3CCN(C(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3F)cc21]

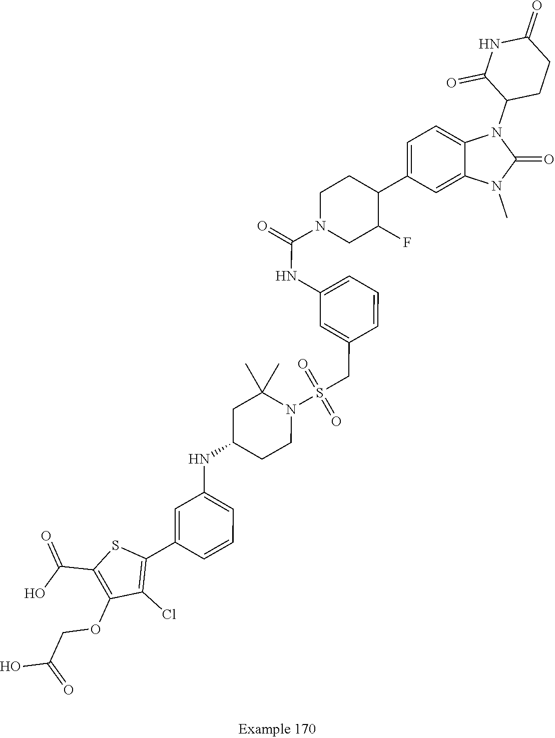 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3F)cc21]

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[3-[4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylate (3, 200 mg, 99.72 μmol, 48% yield, 57% purity, HCl salt) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2, 150 mg, 206.15 μmol) and 3-[5-(3-fluoro-4-piperidyl)-3-methyl-2-oxo-benzimidazol-1-yl]piperidine-2,6-dione (B-396, 101.88 mg, 206.15 μmol, TFA salt) in a similar fashion to Compound B-242. LCMS (ES+): m/z 1107.2 [M+H]⁺

### Step 2: 3-(carboxymethoxy)-4-chloro-5-[3-[[(4S)-1-[[3-[[4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (170)

3-(carboxymethoxy)-4-chloro-5-[3-[[(4S)-1-[3-[4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (170, 70 mg, 62.11 μmol, 62% yield, TFA salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylate (3, 200 mg, 99.72 μmol, HCl salt) in a similar fashion to Compound A-26. The residue was purified by reverse phase prep HPLC [Purification method: Column: XSelect C18 (150×19) mm, 5 micron; Mobile phase A: 0.1% TFA in water; Mobile phase B: MeCN]. LCMS (ES+): m/z 994.0 [M+H]⁺

¹HNMR (400 MHz, DMSO-d6): δ 13.30 (bs, 1H), 11.11 (s, 1H), 8.78 (s, 1H), 7.58 (s, 1H), 7.52-7.49 (m, 1H), 7.29-7.18 (m, 3H), 7.06-6.96 (m, 3H), 6.88 (s, 1H), 6.82 (d, J=7.60 Hz, 1H), 6.70 (d, J=8.40 Hz, 1H), 5.38-5.34 (m, 1H), 4.92 (s, 2H), 4.85-4.55 (m, 3H), 4.40-4.22 (m, 3H), 3.54-3.46 (m, 2H), 3.34 (s, 3H), 3.17-3.14 (m, 1H), 2.94-2.88 (m, 4H), 2.73-2.61 (m, 2H), 2.03-2.01 (m, 1H), 2.00-1.76 (m, 4H), 1.50-1.40 (m, 7H), 1.18-1.05 (m, 1H).

### Example 171

### 3-(carboxymethoxy)-4-chloro-5-[3-[[(4S)-1-[3-[(3R,4R)-4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (171)

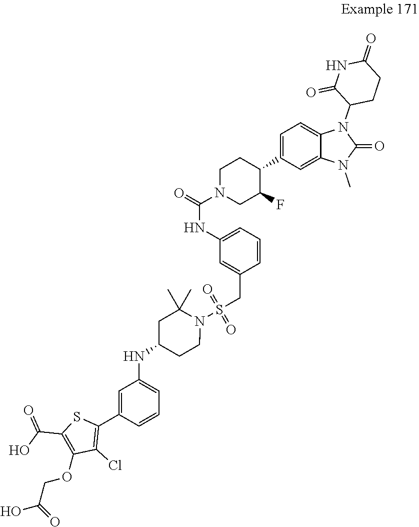 [SMILES: Cn1c(=O)n(C2CCC(=O)NC2=O)c2ccc([C@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)C[C@@H]3F)cc21]

3-(carboxymethoxy)-4-chloro-5-[3-[[(4S)-1-[3-[(3R,4R)-4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (171) was synthesized following the same procedures used for (170).

LCMS (ES+): m/z 994.0 [M+H]⁺

¹HNMR (400 MHz, DMSO-d6): δ 13.20 (bs, 1H), 11.03 (s, 1H), 8.70 (s, 1H), 7.51 (s, 1H), 7.43 (d, J=8.40 Hz, 1H), 7.21-7.11 (m, 3H), 6.99-6.92 (m, 3H), 6.81 (s, 1H), 6.74 (d, J=7.60 Hz, 1H), 6.63 (d, J=8.00 Hz, 1H), 5.31-5.26 (m, 1H), 4.84 (s, 2H), 4.80-4.45 (m, 2H), 4.32-4.15 (m, 5H), 3.47-3.38 (m, 2H), 3.27 (s, 3H), 3.11-3.01 (m, 1H), 2.87-2.81 (m, 3H), 2.65-2.53 (m, 2H), 1.95-1.93 (m, 1H), 1.81-1.69 (m, 4H), 1.43-1.33 (m, 7H), 1.05-0.99 (m, 1H).

### Example 172

### 3-(carboxymethoxy)-4-chloro-5-[3-[[(4S)-1-[3-[(3S,4S)-4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (172)

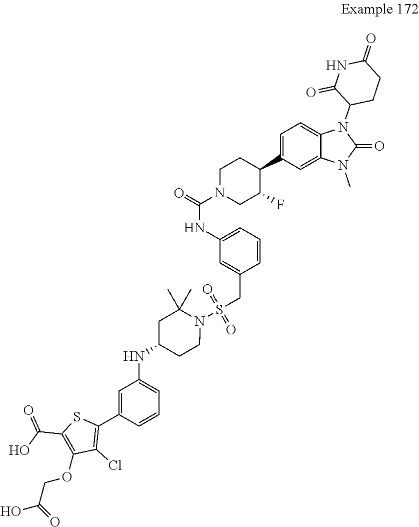 [SMILES: Cn1c(=O)n(C2CCC(=O)NC2=O)c2ccc([C@@H]3CCN(C(=O)Nc4cccc(CS(=O)(=O)N5CC[C@H](Nc6cccc(-c7sc(C(=O)O)c(OCC(=O)O)c7Cl)c6)CC5(C)C)c4)C[C@H]3F)cc21]

3-(carboxymethoxy)-4-chloro-5-[3-[[(4S)-1-[3-[(3S,4S)-4-[1-(2,6-dioxo-3-piperidyl)-3-methyl-2-oxo-benzimidazol-5-yl]-3-fluoro-piperidine-1-carbonyl]amino]phenyl]methylsulfonyl]-2,2-dimethyl-4-piperidyl]amino]phenyl]thiophene-2-carboxylic acid (172) was synthesized following the same procedures used for (170).

LCMS (ES+): m/z 994.0 [M+H]⁺

¹HNMR (400 MHz, DMSO-d6): δ 13.20 (bs, 1H), 11.11 (s, 1H), 8.78 (s, 1H), 7.58 (s, 1H), 7.51 (d, J=8.40 Hz, 1H), 7.29-7.19 (m, 3H), 7.06-7.00 (m, 3H), 6.88 (s, 1H), 6.82 (d, J=8.00 Hz, 1H), 6.70 (d, J=7.20 Hz, 1H), 5.38-5.34 (m, 1H), 4.92 (s, 2H), 4.80-4.55 (m, 2H), 4.40-4.23 (m, 4H), 3.54-3.46 (m, 2H), 3.34 (s, 3H), 3.17-3.14 (m, 1H), 2.94-2.88 (m, 3H), 2.73-2.61 (m, 2H), 2.03-2.00 (m, 1H), 1.89-1.76 (m, 4H), 1.49-1.40 (m, 7H), 1.15-1.05 (m, 1H).

### Example 173

### 3-(carboxymethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylic acid (173)

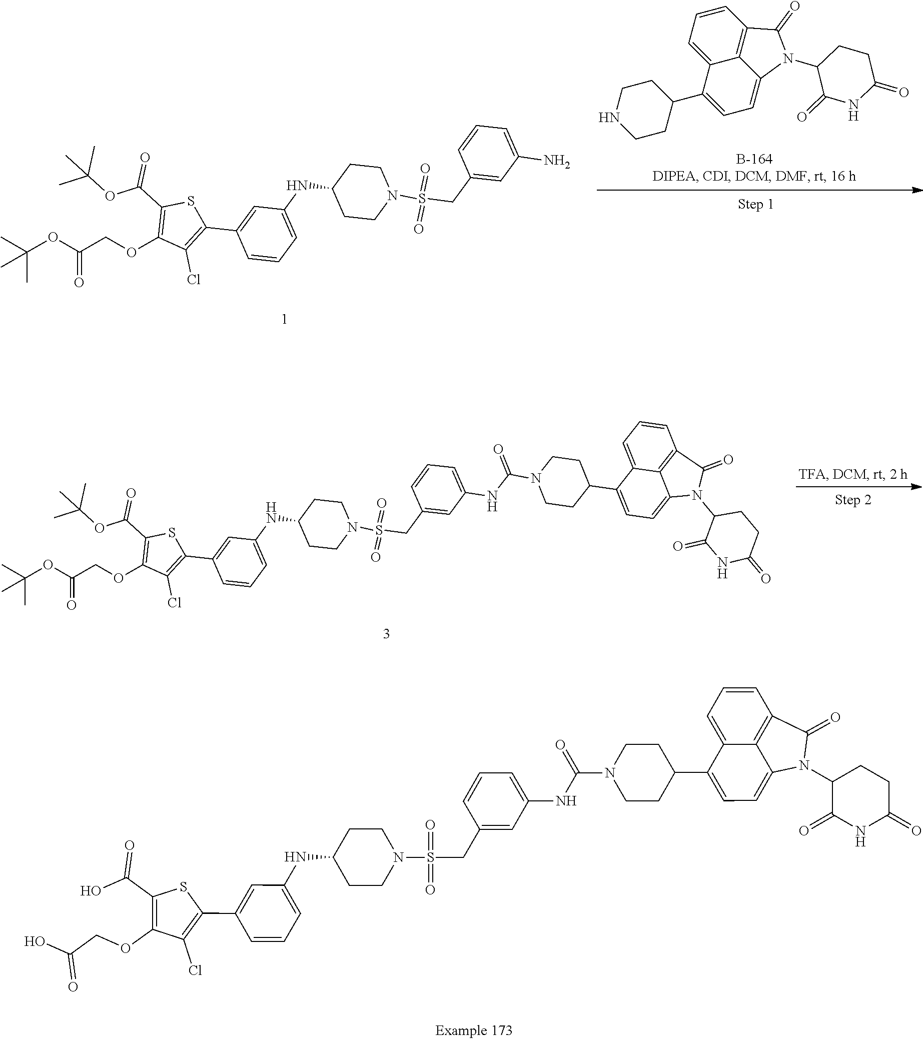

### Step 1: tert-butyl 3-(2-tert-butoxy-2-oxo-ethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylate (3)

tert-butyl 3-(2-tert-butoxy-2-oxo-ethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylate (3, 220 mg, 133.77 μmol, 66% yield, 68% purity, HCl salt) was synthesized from tert-butyl 5-[3-[[1-[(3-aminophenyl)methylsulfonyl]-4-piperidyl]amino]phenyl]-3-(2-tert-butoxy-2-oxo-ethoxy)-4-chloro-thiophene-2-carboxylate (1, 150 mg, 201.51 μmol) and 3-[2-oxo-6-(4-piperidyl)benzo[cd]indol-1-yl]piperidine-2,6-dione (B-194, 78.68 mg, 153.38 μmol, TFA salt) in a similar fashion to Compound B-242.

LCMS (ES+): m/z 1081.2 [M+H]⁺

### Step 2: 3-(carboxymethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylic acid (173)

3-(carboxymethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylic acid (173, 80 mg, 72.17 μmol, 54% yield, TFA salt) was synthesized from tert-butyl 3-(2-tert-butoxy-2-oxo-ethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylate (3, 220 mg, 133.77 μmol, HCl salt) in a similar fashion to Compound A-26. The residue was purified by reverse phase prep HPLC [Purification method: Column: XSelect C18 (150×19) mm, 5 micron; Mobile phase A: 0.1% TFA in water; Mobile phase B: MeCN] to afford 3-(carboxymethoxy)-4-chloro-5-[3-[[1-[[3-[[4-[1-(2,6-dioxo-3-piperidyl)-2-oxo-benzo[cd]indol-6-yl]piperidine-1-carbonyl]amino]phenyl]methylsulfonyl]-4-piperidyl]amino]phenyl]thiophene-2-carboxylic acid (173, 80 mg, 72.17 μmol, 54% yield, TFA salt) as a yellow solid. LCMS (ES+): m/z 969.0 [M+H]⁺

¹HNMR (400 MHz, DMSO-d6): δ 13.50 (bs, 1H), 11.13 (s, 1H), 8.68 (s, 1H), 8.50 (d, J=8.40 Hz, 1H), 8.12 (d, J=6.80 Hz, 1H), 7.89 (dd, J=7.20, 8.00 Hz, 1H), 7.61 (s, 1H), 7.45 (d, J=9.20 Hz, 1H), 7.39 (d, J=7.60 Hz, 1H), 7.26 (t, J=8.00 Hz, 1H), 7.21 (t, J=8.00 Hz, 1H), 7.10 (d, J=7.60 Hz, 1H), 7.00 (d, J=7.60 Hz, 1H), 6.89 (s, 1H), 6.82 (d, J=7.60 Hz, 1H), 6.72 (dd, J=1.60, 8.20 Hz, 1H), 5.47-5.43 (m, 1H), 4.92 (s, 2H), 4.38-4.35 (m, 4H), 3.76-3.45 (m, 4H), 3.10-2.93 (m, 5H), 2.77-2.64 (m, 2H), 2.11-2.08 (m, 1H), 1.96-1.90 (m, 4H), 1.78-1.68 (m, 2H), 1.45-1.35 (m, 2H).

### Example 174

3-(carboxymethoxy)-4-chloro-5-[3-[[(4S)-1-[[3-[2-[1-[1-(2,6-dioxo-3-piperidyl)-3-methyl-2-oxo-benzimidazol-5-yl]-4-piperidyl]ethylcarbamoylamino]phenyl]methylsulfonyl]-2,2-dimethyl-4-piperidyl]amino]phenyl]thiophene-2-carboxylic acid (174)

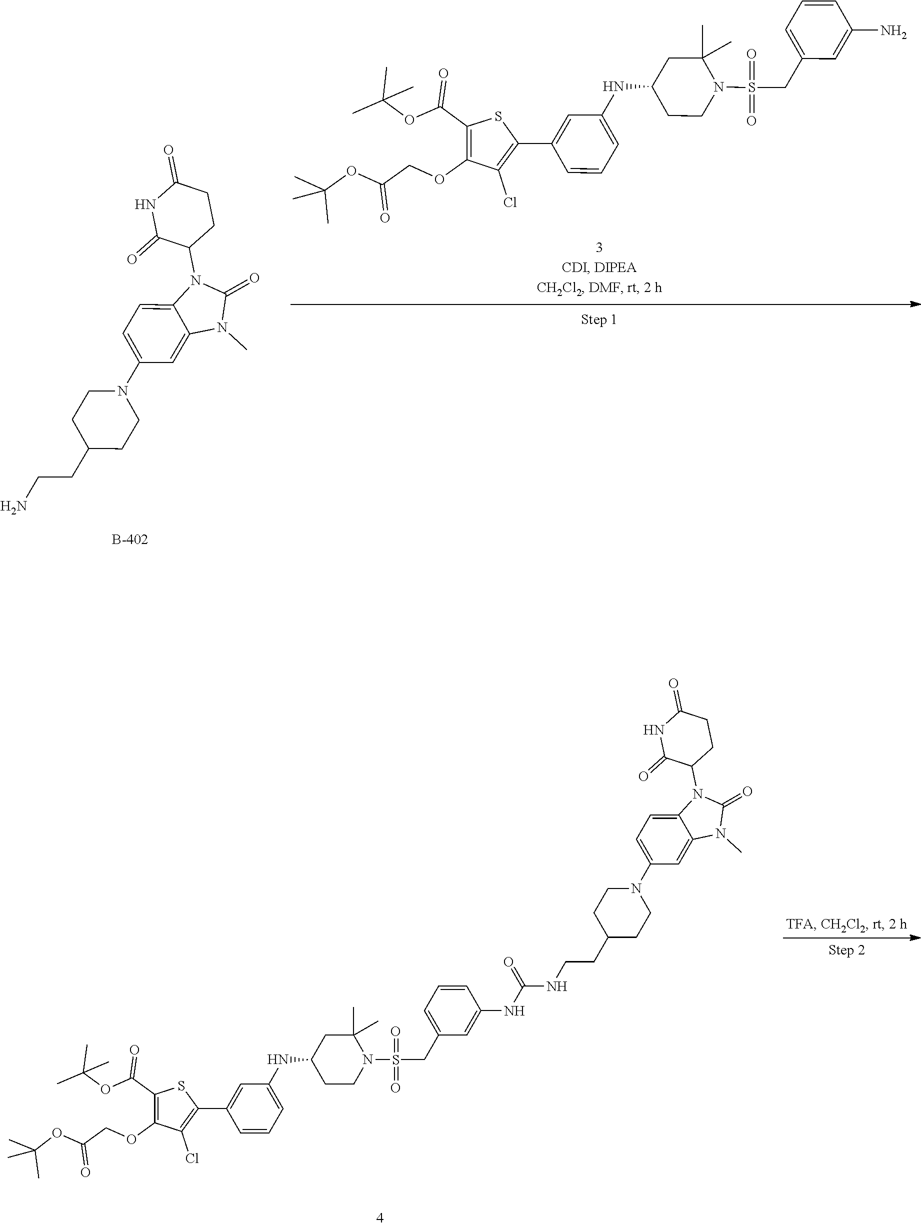 [SMILES: Cn1c(=O)n(C2CCC(=O)NC2=O)c2ccc(N3CCC(CCN)CC3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(N3CCC(CCNC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3)cc21]

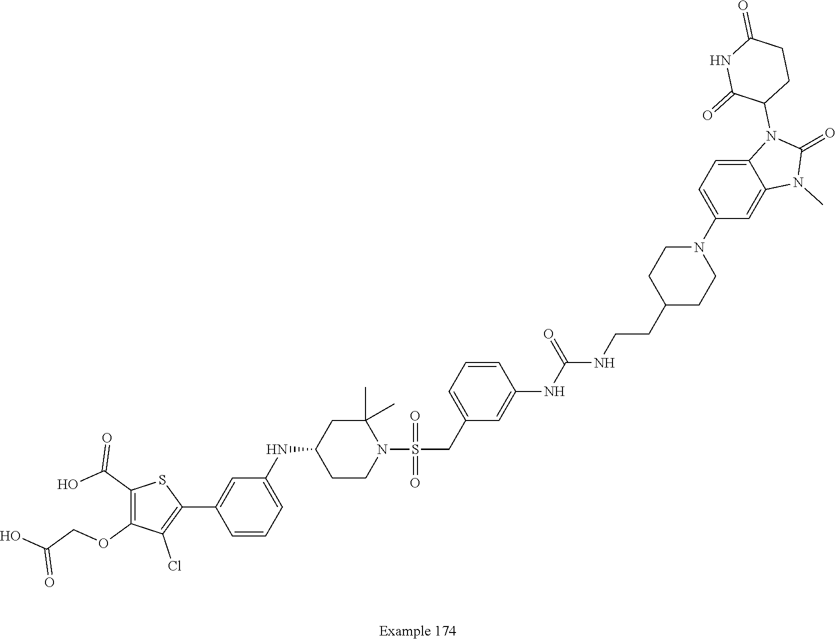 [SMILES: Cn1c(=O)n(C2CCC(=O)NC2=O)c2ccc(N3CCC(CCNC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: tert-butyl 3-(2-tert-butoxy-2-oxo-ethoxy)-4-chloro-5-[3-[[(4S)-1-[[3-[2-[1-[1-(2,6-dioxo-3-piperidyl)-3-methyl-2-oxo-benzimidazol-5-yl]-4-piperidyl]ethylcarbamoylamino]phenyl]methylsulfonyl]-2,2-dimethyl-4-piperidyl]amino]phenyl]thiophene-2-carboxylate (4)

tert-butyl 3-(2-tert-butoxy-2-oxo-ethoxy)-4-chloro-5-[3-[[(4S)-1-[[3-[2-[1-[1-(2,6-dioxo-3-piperidyl)-3-methyl-2-oxo-benzimidazol-5-yl]-4-piperidyl]ethylcarbamoylamino]phenyl]methylsulfonyl]-2,2-dimethyl-4-piperidyl]amino]phenyl]thiophene-2-carboxylate (4, 250 mg, 209.93 μmol, 88% yield) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2, 200 mg, 239.70 μmol, TFA salt) and 3-[5-[4-(2-aminoethyl)-1-piperidyl]-3-methyl-2-oxo-benzimidazol-1-yl]piperidine-2,6-dione (B-402, 181.28 mg, 359.56 μmol, TFA salt) in a similar fashion to Compound B-242.

LCMS (ES+): m/z 1133.2 [M+H]⁺

### Step 2: 3-(carboxymethoxy)-4-chloro-5-[3-[[(4S)-1-[[3-[2-[1-[1-(2,6-dioxo-3-piperidyl)-3-methyl-2-oxo-benzimidazol-5-yl]-4-piperidyl]ethylcarbamoylamino]phenyl]methylsulfonyl]-2,2-dimethyl-4-piperidyl]amino]phenyl]thiophene-2-carboxylic acid (174)

3-(carboxymethoxy)-4-chloro-5-[3-[[(4S)-1-[3-[2-[1-[1-(2,6-dioxo-3-piperidyl)-3-methyl-2-oxo-benzimidazol-5-yl]-4-piperidyl]ethylcarbamoylamino]phenyl]methylsulfonyl]-2,2-dimethyl-4-piperidyl]amino]phenyl]thiophene-2-carboxylic acid (174, 120 mg, 108.90 μmol, 85% yield, formic acid salt) was synthesized from tert-butyl 3-(2-tert-butoxy-2-oxo-ethoxy)-4-chloro-5-[3-[[(4S)-1-[[3-[2-[1-[1-(2,6-dioxo-3-piperidyl)-3-methyl-2-oxo-benzimidazol-5-yl]-4-piperidyl]ethylcarbamoylamino]phenyl]methylsulfonyl]-2,2-dimethyl-4-piperidyl]amino]phenyl]thiophene-2-carboxylate (4, 150 mg, 127.54 μmol) in a similar fashion to Compound A-26. The residue was purified with reverse phase preparatory HPLC [Purification method: Column: XSelect C18 (250×19) mm, 5 micron; Mobile phase A: 0.1% Formic acid in water; Mobile phase B: MeCN]. LCMS (ES+): m/z 1019.8 [M+H]⁺1H-NMR (400 MHz, DMSO-d6): δ 11.07 (s, 1H), 8.52 (s, 1H), 7.47 (s, 1H), 7.39 (d, J=8.00 Hz, 1H), 7.38-7.18 (m, 2H), 6.96-6.93 (m, 2H), 6.87 (s, 2H), 6.82-6.80 (m, 1H), 6.71-6.66 (m, 2H), 6.16 (t, J=5.20 Hz, 1H), 5.64 (s, 1H), 5.32-5.27 (m, 1H), 4.90 (s, 2H), 3.59-3.42 (m, 5H), 3.31 (s, 3H), 3.19-3.09 (m, 4H), 2.95-2.82 (m, 1H), 2.70-2.64 (m, 4H), 2.00-1.97 (m, 1H), 1.85-1.78 (m, 4H), 1.45-1.30 (m, 11H), 1.15-1.05 (m, 1H).

### Example 175

### 3-(carboxymethoxy)-4-chloro-5-[3-[[(4S)-1-[[3-[[4-[[1-(2,6-dioxo-3-piperidyl)-3-methyl-2-oxo-benzimidazol-5-yl]methyl]piperidine-1-carbonyl]amino]phenyl]methylsulfonyl]-2,2-dimethyl-4-piperidyl]amino]phenyl]thiophene-2-carboxylic acid (175)

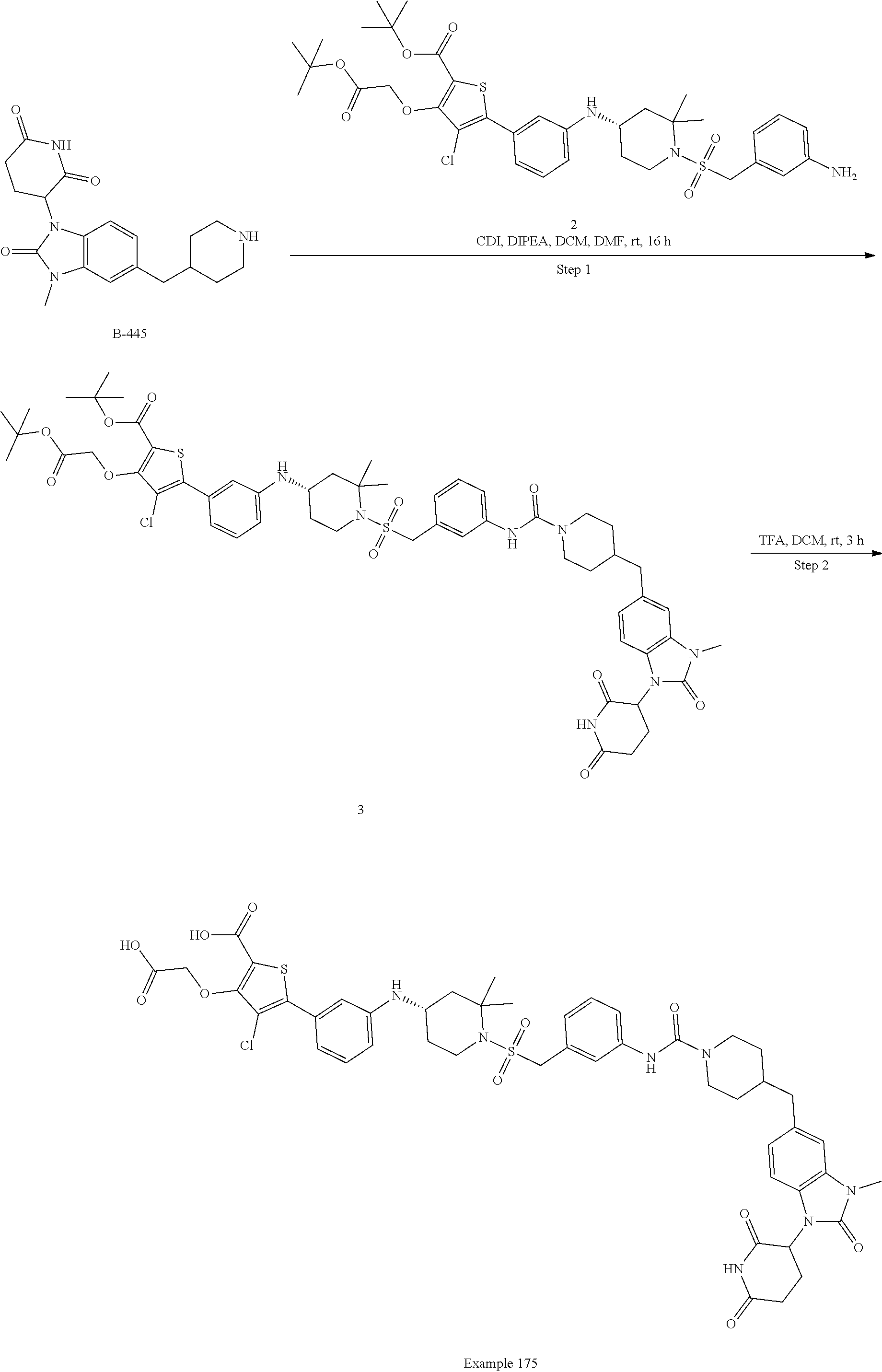 [SMILES: Cn1c(=O)n(C2CCC(=O)NC2=O)c2ccc(CC3CCNCC3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(CC3CCN(C(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3)cc21 ; Cn1c(=O)n(C2CCC(=O)NC2=O)c2ccc(CC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21]

### Step 1: tert-butyl 3-(2-tert-butoxy-2-oxo-ethoxy)-4-chloro-5-[3-[[(4S)-1-[[3-[[4-[[1-(2,6-dioxo-3-piperidyl)-3-methyl-2-oxo-benzimidazol-5-yl]methyl]piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[3-[4-[[1-(2,6-dioxo-3-piperidyl)-3-methyl-2-oxo-benzimidazol-5-yl]methyl]piperidine-1-carbonyl]amino]phenyl]methylsulfonyl]-2,2-dimethyl-4-piperidyl]amino]phenyl]thiophene-2-carboxylate (3, 260 mg, 180.74 μmol, 77% yield, 77% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxy late (2, 170 mg, 235.29 μmol) and 3-[3-methyl-2-oxo-5-(4-piperidylmethyl)benzimidazol-1-yl]piperidine-2,6-dione (B-445, 93.20 mg, 213.46 μmol, HCl salt) in a similar fashion to Compound B-242. LCMS (ES−): m/z 1100.0 [M−H]⁻

### Step 2: 3-(carboxymethoxy)-4-chloro-5-[3-[[(4S)-1-[[3-[[4-[[1-(2,6-dioxo-3-piperidyl)-3-methyl-2-oxo-benzimidazol-5-yl]methyl]piperidine-1-carbonyl]amino]phenyl]methylsulfonyl]-2,2-dimethyl-4-piperidyl]amino]phenyl]thiophene-2-carboxylic acid (175)

3-(carboxymethoxy)-4-chloro-5-[3-[[(4S)-1-[[3-[[4-[[1-(2,6-dioxo-3-piperidyl)-3-methyl-2-oxo-benzimidazol-5-yl]methyl]piperidine-1-carbonyl]amino]phenyl]methylsulfonyl]-2,2-dimethyl-4-piperidyl]amino]phenyl]thiophene-2-carboxylic acid (175, 41 mg, 35.86 μmol, 17% yield, TFA salt) was synthesized from tert-butyl 3-(2-tert-butoxy-2-oxo-ethoxy)-4-chloro-5-[3-[[(4S)-1-[[3-[[4-[[1-(2,6-dioxo-3-piperidyl)-3-methyl-2-oxo-benzimidazol-5-yl]methyl]piperidine-1-carbonyl]amino]phenyl]methylsulfonyl]-2,2-dimethyl-4-piperidyl]amino]phenyl]thiophene-2-carboxylate (3, 300 mg, 208.55 μmol) in in a similar fashion to Compound A-26. The residue was purified by reverse phase prep HPLC [Purification method: XBridge C18 (19.1×150) mm; Mobile phase A: 0.1% TFA in water; Mobile phase B: Acetonitrile].

LCMS (ES+): m/z 990.2 [M+H]⁺

¹HNMR (400 MHz, DMSO-d6): δ 13.20 (bs, 1H), 11.09 (s, 1H), 8.54 (s, 1H), 7.54 (s, 1H), 7.42 (d, J=8.00 Hz, 1H), 7.25-7.18 (m, 2H), 7.03-6.96 (m, 3H), 6.86-6.80 (m, 3H), 6.68 (d, J=8.40 Hz, 1H), 5.67 (bs, 1H), 5.37-5.32 (m, 1H), 4.92 (s, 2H), 4.29 (dd, J=13.60, 45.20 Hz, 2H), 4.12 (d, J=13.60 Hz, 2H), 3.44-3.36 (m, 1H), 3.26 (s, 3H), 3.15-3.09 (m, 1H), 2.95-2.85 (m, 1H), 2.73-2.67 (m, 4H), 2.05-1.98 (m, 1H), 1.86-1.65 (m, 3H), 1.62-1.55 (m, 2H), 1.47 (s, 3H), 1.39 (s, 3H), 1.18-1.02 (m, 3H).

### Example 176

### 3-(carboxymethoxy)-4-chloro-5-[3-[[(4S)-1-[[3-[[2-[4-[1-(2,6-dioxo-3-piperidyl)-3-isopropyl-2-oxo-benzimidazol-5-yl]-3,3-difluoro-1-piperidyl]acetyl]amino]phenyl]methylsulfonyl]-2,2-dimethyl-4-piperidyl]amino]phenyl]thiophene-2-carboxylic acid (176)

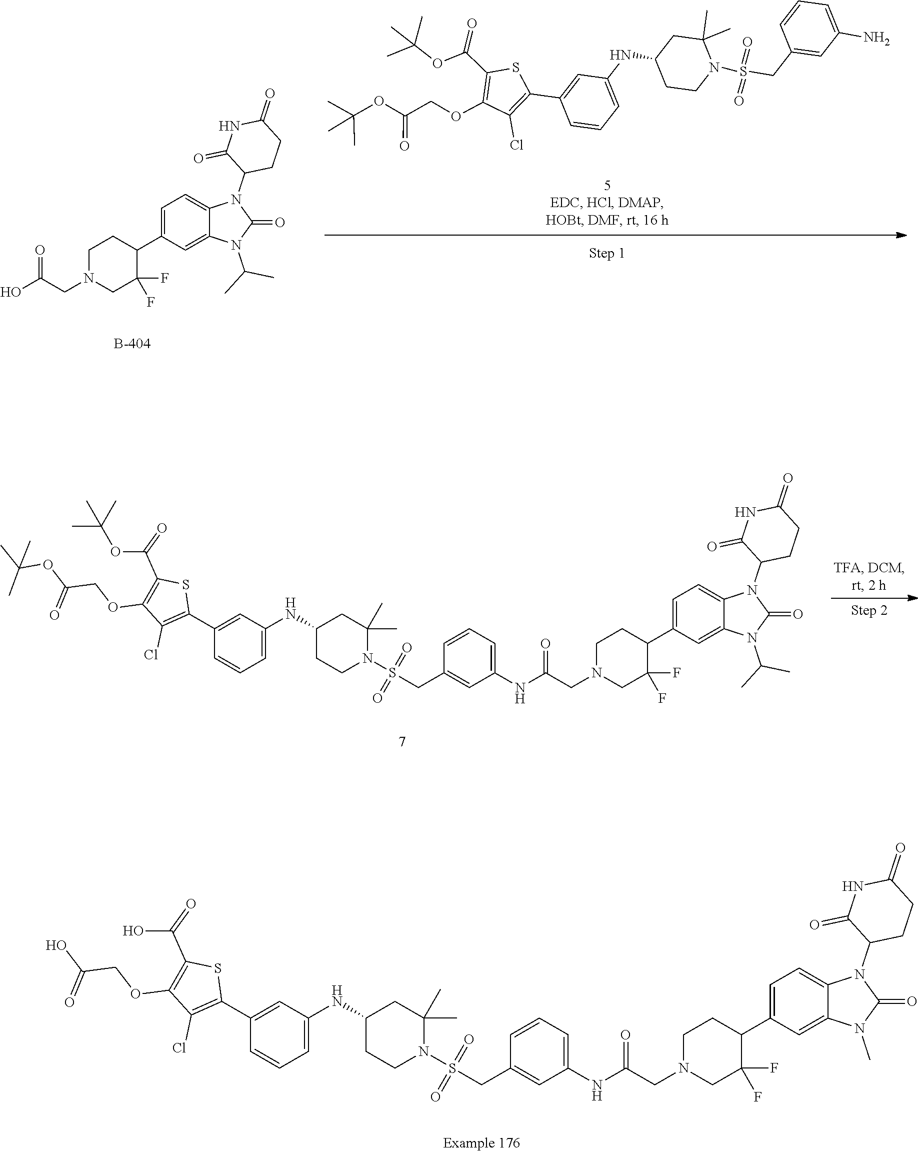 [SMILES: CC(C)n1c(=O)n(C2CCC(=O)NC2=O)c2ccc(C3CCN(CC(=O)O)CC3(F)F)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)n1c(=O)n(C2CCC(=O)NC2=O)c2ccc(C3CCN(CC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3(F)F)cc21 ; Cn1c(=O)n(C2CCC(=O)NC2=O)c2ccc(C3CCN(CC(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21]

### Step 1: tert-butyl 3-(2-tert-butoxy-2-oxo-ethoxy)-4-chloro-5-[3-[[(4S)-1-[[3-[[2-[4-[1-(2,6-dioxo-3-piperidyl)-3-isopropyl-2-oxo-benzimidazol-5-yl]-3,3-difluoro-1-piperidyl]acetyl]amino]phenyl]methylsulfonyl]-2,2-dimethyl-4-piperidyl]amino]phenyl]thiophene-2-carboxylate (6)

tert-butyl 3-(2-tert-butoxy-2-oxo-ethoxy)-4-chloro-5-[3-[[(4S)-1-[[3-[[2-[4-[1-(2,6-dioxo-3-piperidyl)-3-isopropyl-2-oxo-benzimidazol-5-yl]-3,3-difluoro-1-piperidyl]acetyl]amino]phenyl]methylsulfonyl]-2,2-dimethyl-4-piperidyl]amino]phenyl]thiophene-2-carboxylate (6, 140 mg, 80.39 μmol, 84% yield, 67% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (5, 70 mg, 96.20 μmol) and 2-[4-[1-(2,6-dioxo-3-piperidyl)-3-isopropyl-2-oxo-benzimidazol-5-yl]-3,3-difluoro-1-piperidyl]acetic acid (B-404, 64.71 mg, 96.20 μmol, TFA salt in a similar fashion to Example 166, except with the addition of 1.5 eq. HOBt.

LCMS (ES+): m/z 1167.2 [M+H]⁺

### Step 2: 3-(carboxymethoxy)-4-chloro-5-[3-[[(4S)-1-[[3-[2-[4-[1-(2,6-dioxo-3-piperidyl)-3-isopropyl-2-oxo-benzimidazol-5-yl]-3,3-difluoro-1-piperidyl]acetyl]amino]phenyl]methylsulfonyl]-2,2-dimethyl-4-piperidyl]amino]phenyl]thiophene-2-carboxylic acid (176)

3-(carboxymethoxy)-4-chloro-5-[3-[[(4S)-1-[3-[2-[4-[1-(2,6-dioxo-3-piperidyl)-3-isopropyl-2-oxo-benzimidazol-5-yl]-3,3-difluoro-1-piperidyl]acetyl]amino]phenyl]methylsulfonyl]-2,2-dimethyl-4-piperidyl]amino]phenyl]thiophene-2-carboxylic acid (176, 40 mg, 33.11 μmol, 41% yield, TFA salt) was synthesized from tert-butyl 3-(2-tert-butoxy-2-oxo-ethoxy)-4-chloro-5-[3-[[(4S)-1-[3-[2-[4-[1-(2,6-dioxo-3-piperidyl)-3-isopropyl-2-oxo-benzimidazol-5-yl]-3,3-difluoro-1-piperidyl]acetyl]amino]phenyl]methylsulfonyl]-2,2-dimethyl-4-piperidyl]amino]phenyl]thiophene-2-carboxylate (6, 140 mg, 80.39 μmol) in a similar fashion to Compound A-26. The residue was purified by reverse phase prep HPLC [Purification method: Column: X-Select, C18 (150×19) mm, 5 micron; Mobile phase A: 0.1% TFA in water; Mobile phase B: MeCN].

LCMS (ES+): m/z 1054.2 [M+H]⁺

1H-NMR (400 MHz, DMSO-d6): δ 13.25 (s, 1H), 11.11 (s, 1H), 10.20 (s, 1H), 7.71 (t, J=Hz, 1H), 7.64 (d, J=8.00 Hz, 1H), 7.37 (t, J=8.00 Hz, 1H), 7.24-7.14 (m, 3H), 7.09 (d, J=8.00 Hz, 1H), 6.98 (d, J=8.00 Hz, 1H), 6.88 (s, 1H), 6.81 (d, J=7.60 Hz, 1H), 6.71 (d, J=8.40 Hz, 1H), 5.38-5.33 (m, 1H), 4.92 (s, 2H), 4.66-4.59 (m, 1H), 4.39 (dd, J=13, 60, 47.20 Hz, 2H), 3.27-3.21 (m, 1H), 3.17-3.11 (m, 2H), 2.94-2.87 (m, 1H), 2.71-2.55 (m, 3H), 2.08-1.79 (m, 4H), 1.48-1.37 (m, 14H), 1.15-1.04 (m, 1H).

### Example 177

### 3-(carboxymethoxy)-4-chloro-5-[3-[[(4S)-1-[3-[3-[[1-(2,6-dioxo-3-piperidyl)-2-oxo-benzo[cd]indol-5-yl]propylcarbamoylamino]phenyl]methylsulfonyl]-2,2-dimethyl-4-piperidyl]amino]phenyl]thiophene-2-carboxylic acid (177)

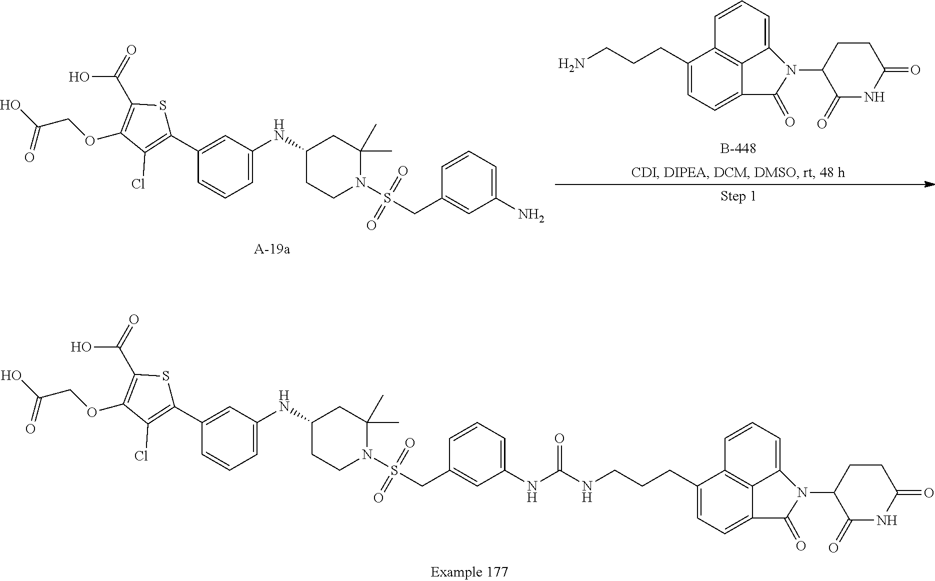 [SMILES: CC1(C)C[C@@H](Nc2cccc(-c3sc(C(=O)O)c(OCC(=O)O)c3Cl)c2)CCN1S(=O)(=O)Cc1cccc(N)c1 ; NCCCc1ccc2c3c(cccc13)N(C1CCC(=O)NC1=O)C2=O ; CC1(C)C[C@@H](Nc2cccc(-c3sc(C(=O)O)c(OCC(=O)O)c3Cl)c2)CCN1S(=O)(=O)Cc1cccc(NC(=O)NCCCc2ccc3c4c(cccc24)N(C2CCC(=O)NC2=O)C3=O)c1]

### Step 1: 3-(carboxymethoxy)-4-chloro-5-[3-[[(4S)-1-[[3-[3-[[1-(2,6-dioxo-3-piperidyl)-2-oxo-benzo[cd]indol-5-yl]propylcarbamoylamino]phenyl]methylsulfonyl]-2,2-dimethyl-4-piperidyl]amino]phenyl]thiophene-2-carboxylic acid (177)

3-(carboxymethoxy)-4-chloro-5-[3-[[(4S)-1-[3-[3-[[1-(2,6-dioxo-3-piperidyl)-2-oxo-benzo[cd]indol-5-yl]propylcarbamoylamino]phenyl]methylsulfonyl]-2,2-dimethyl-4-piperidyl]amino]phenyl]thiophene-2-carboxylic acid (177, 14 mg, 11.92 μmol, 3% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 305.01 mg, 401.25 μmol, TFA salt) and 3-(5-(3-aminopropyl)-2-oxobenzo[cd]indol-[(2H)-yl)piperidine-2,6-dione (B-448, 150 mg, 401.25 μmol, HCl salt) in a similar fashion to Compound B-242. The residue was purified by reverse phase prep HPLC [Purification method: Column: Phenomemex Luna C18 (250×21.2) mm; 5 micron; Mobile phase A: 0.1% TFA in water and Mobile phase B: MeCN].

LCMS (ES+): m/z 972.0 [M+H]⁺

¹HNMR (400 MHz, DMSO-d6): δ 11.12 (s, 1H), 8.59 (s, 1H), 8.00 (d, J=7.20 Hz, 1H), 7.77 (d, J=8.40 Hz, 1H), 7.67 (d, J=7.60 Hz, 1H), 7.53 (t, J=8.40 Hz, 1H), 7.47 (s, 1H), 7.40 (d, J=8.40 Hz, 1H), 7.26-7.13 (m, 5H), 7.01 (s, 1H), 6.94 (d, J=7.60 Hz, 1H), 6.86 (s, 1H), 6.80 (d, J=7.60 Hz, 1H), 6.68 (dd, J=1.60, 8.00 Hz, 1H), 6.33 (s, 1H), 5.45-5.35 (m, 1H), 4.91 (s, 2H), 4.30 (dd, J=14, 00, 47.00 Hz, 2H), 3.22-3.08 (m, 4H), 2.99-2.90 (m, 1H), 2.76-2.64 (m, 2H), 2.15-2.07 (m, 1H), 1.90-1.76 (m, 4H), 1.45-1.38 (m, 8H), 1.15-1.08 (m, 1H).

### Example 178

### 3-(carboxymethoxy)-4-chloro-5-(3-(((4S)-1-((3-(2-(4-(1-(2,6-dioxopiperidin-3-yl)-3-methyl-2-oxo-2,3-dihydro-1H-benzo[d]imidazol-5-yl)-3-fluorophenyl)acetamido)benzyl)sulfonyl)-2,2-dimethylpiperidin-4-yl)amino)phenyl)thiophene-2-carboxylic acid (178)

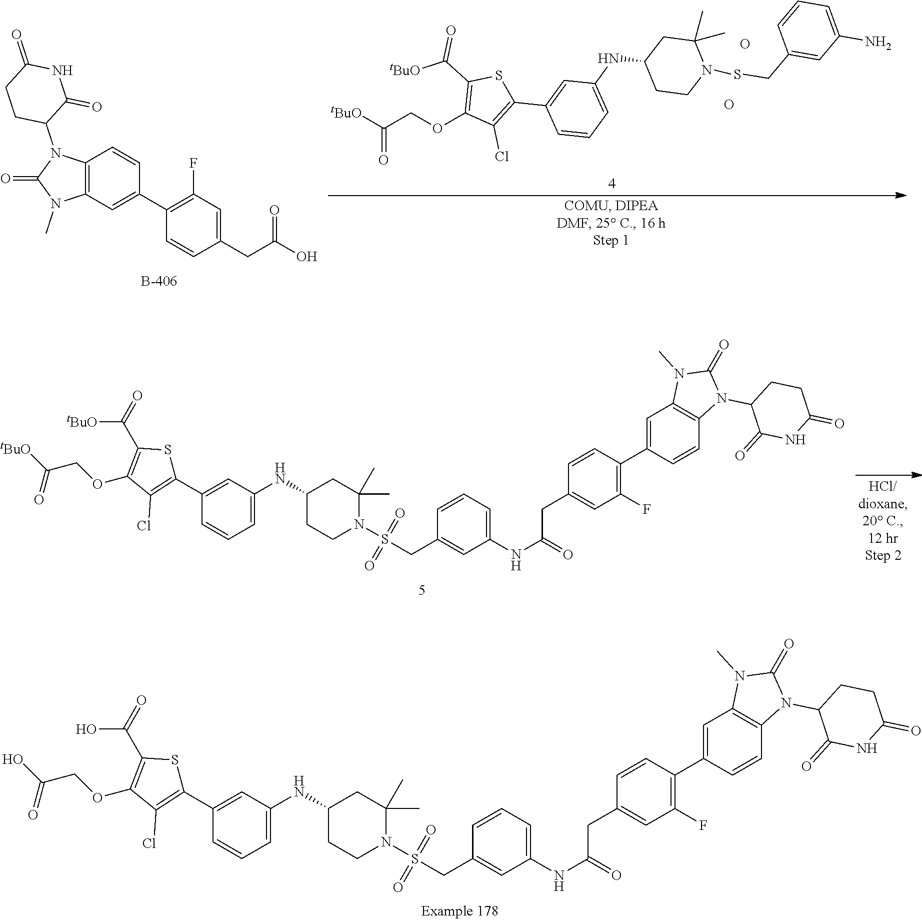

### Step 1: tert-butyl 3-(2-(tert-butoxy)-2-oxoethoxy)-4-chloro-5-(3-(((4S)-1-((3-(2-(4-(1-(2,6-dioxopiperidin-3-yl)-3-methyl-2-oxo-2,3-dihydro-1H-benzo[d]imidazol-5-yl)-3-fluorophenyl)acetamido)benzyl)sulfonyl)-2,2-dimethylpiperidin-4-yl)amino)phenyl)thiophene-2-carboxylate (5)

To a solution of(S)-tert-butyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (4, 122.57 mg, 170.16 μmol) in DMF (2 mL) was added 2-(4-(1-(2,6-dioxopiperidin-3-yl)-3-methyl-2-oxo-2,3-dihydro-1H-benzo[d]imidazol-5-yl)-3-fluorophenyl)acetic acid (B-406, 70 mg, 170.16 μmol), DIPEA (109.96 mg, 850.79 μmol, 148.19 μL) and COMU (80.16 mg, 187.17 μmol). The mixture was stirred at 25° C. for 12 h. The reaction mixture was diluted with brine (20 mL) and extracted with EtOAc (10 mL×3). The combined organic layers were washed with brine (10 mL×3), dried over Na₂SO₄, filtered and concentrated under reduced pressure. The residue was purified by prep-TLC (SiO2, petroleum ether/ethyl acetate=2/1) to afford tert-butyl 3-(2-(tert-butoxy)-2-oxoethoxy)-4-chloro-5-(3-(((4S)-1-((3-(2-(4-(1-(2,6-dioxopiperidin-3-yl)-3-methyl-2-oxo-2,3-dihydro-1H-benzo[d]imidazol-5-yl)-3-fluorophenyl)acetamido)benzyl)sulfonyl)-2,2-dimethylpiperidin-4-yl)amino)phenyl)thiophene-2-carboxylate (120 mg, 107.75 μmol, 63% yield) as a white solid.

### Step 2: 3-(carboxymethoxy)-4-chloro-5-(3-(((4S)-1-((3-(2-(4-(1-(2,6-dioxopiperidin-3-yl)-3-methyl-2-oxo-2,3-dihydro-1H-benzo[d]imidazol-5-yl)-3-fluorophenyl)acetamido)benzyl)sulfonyl)-2,2-dimethylpiperidin-4-yl)amino)phenyl)thiophene-2-carboxylic acid (178)

A solution of tert-butyl 3-(2-(tert-butoxy)-2-oxoethoxy)-4-chloro-5-(3-(((4S)-1-((3-(2-(4-(1-(2,6-dioxopiperidin-3-yl)-3-methyl-2-oxo-2,3-dihydro-1H-benzo[d]imidazol-5-yl)-3-fluorophenyl)acetamido)benzyl)sulfonyl)-2,2-dimethylpiperidin-4-yl)amino)phenyl)thiophene-2-carboxylate (5, 100 mg, 89.79 μmol) in HCl/dioxane (3 mL, 4M). The mixture was stirred at 20° C. for 12 h. The reaction mixture was concentrated under reduced pressure to remove dioxane. The residue was purified by reversed phase flash chromatography (flow: 60 mL/min; gradient: from 10-55% MeCN in water (0.1% TFA) over 12 min; column: Welch Ultimate XB-C18, 20-40 μm, 100 Å, I.D. 95 mm× H 365 mm) to afford 3-(carboxymethoxy)-4-chloro-5-(3-(((4S)-1-((3-(2-(4-(1-(2,6-dioxopiperidin-3-yl)-3-methyl-2-oxo-2,3-dihydro-1H-benzo[d]imidazol-5-yl)-3-fluorophenyl)acetamido)benzyl)sulfonyl)-2,2-dimethylpiperidin-4-yl)amino)phenyl)thiophene-2-carboxylic acid (178, 26.1 mg, 22.46 μmol, 25% yield, TFA salt) as a white solid. LCMS (ESI): m/z 1001.3 [M+H]⁺

1H NMR (400 MHz, d₆-DMSO) δ 11.14 (s, 1H), 10.31 (s, 1H), 7.68 (s, 1H), 7.64 (d, J=8.4 Hz, 1H), 7.49 (t, J=8.4 Hz, 1H), 7.37-7.32 (m, 2H), 7.31-7.25 (m, 2H), 7.22-7.17 (m, 3H), 7.10 (d, J=7.2 Hz, 1H), 6.88 (s, 1H), 6.81 (d, J=7.6 Hz, 1H), 6.71-6.63 (m, 1H), 5.42 (dd, J=5.6, 12.0 Hz, 1H), 4.91 (s, 2H), 4.42 (d, J=14.4 Hz, 1H), 4.30 (d, J=13.6 Hz, 1H), 3.74 (s, 2H), 3.38 (s, 3H), 3.12 (s, 1H), 2.91 (d, J=12 Hz, 1H), 2.77 (d, J=4.0 Hz, 1H), 2.62 (s, 1H), 2.09-2.03 (m, 1H), 1.90-1.84 (m, 1H), 1.82-1.76 (m, 1H), 1.50-1.33 (m, 8H), 1.27-1.09 (m, 2H).

### Example 179

### 3-(carboxymethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylic acid (179)

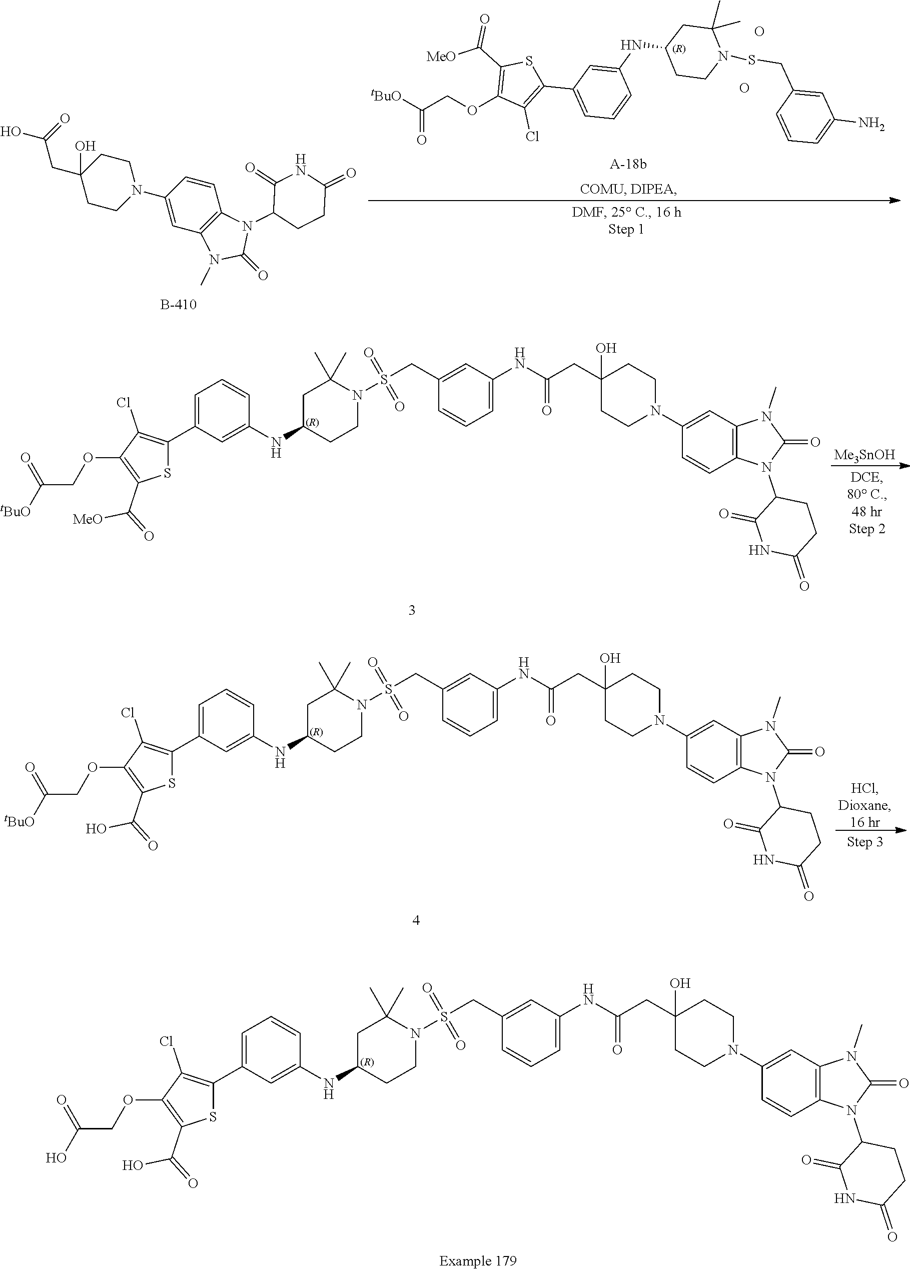

### Step 1: methyl 3-(2-(tert-butoxy)-2-oxoethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylate (3)

methyl 3-(2-(tert-butoxy)-2-oxoethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylate (3, 0.3 g, 250.77 μmol, 52% yield) was synthesized from 2-(1-(1-(2,6-dioxopiperidin-3-yl)-3-methyl-2-oxo-2,3-dihydro-1H-benzo[d]imidazol-5-yl)-4-hydroxypiperidin-4-yl)acetic acid (B-410, 200 mg, 480.28 μmol) and (R)-methyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (A-18b, 325.75 mg, 480.28 μmol) in a similar fashion to Example 178. The residue was purified by column chromatography (SiO₂, petroleum ether/ethyl acetate=10/1 to 1/5).

LCMS (ESI): m/z 1076.4 [M+H]⁺

### Step 2: 3-(2-(tert-butoxy)-2-oxoethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylic acid (4)

3-(2-(tert-butoxy)-2-oxoethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylic acid (4, 260 mg, 21% purity) was synthesized from methyl 3-(2-(tert-butoxy)-2-oxoethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylate (3, 200 mg, 185.76 μmol) in a similar fashion to Compound B-11. The mixture was used for next step without further purification.

LCMS (ESI): m/z 1062.4 [M+H]⁺

### Step 3: 3-(carboxymethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylic acid (179)

3-(carboxymethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylic acid (179, 3.59 mg, 3.17 μmol, 2% yield) was synthesized from methyl 3-(2-(tert-butoxy)-2-oxoethoxy)-4-chloro-5-(3-(((4R)-1-((3-(2-(1-(1-(2,6-dioxopiperidin-3-yl)-3-methyl-2-oxo-2,3-dihydro-1H-benzo[d]imidazol-5-yl)-4-hydroxypiperidin-4-yl)acetamido)benzyl)sulfonyl)-2,2-dimethylpiperidin-4-yl)amino)phenyl)thiophene-2-carboxylic acid (4, 200 mg, 21% purity) in a similar fashion to step 2 of Example 178. The residue was purified by prep-HPLC (flow: 30 mL/min; gradient: from 0-25% MeCN in water (0.1% TFA) over 10 min; column: Phenomenex Synergi C18 150×25 mm×10 μm). LCMS (ESI): m/z 1006.4 [M+H]⁺

¹H NMR (400 MHz, d₆-DMSO) δ 11.60 (s, 1H), 10.12-10.01 (m, 1H), 7.72 (s, 1H), 7.60 (d, J=8.0 Hz, 1H), 7.35-7.31 (m, 1H), 7.27 (s, 1H), 7.21-7.17 (m, 1H), 7.14-7.09 (m, 2H), 7.06-7.00 (m, 1H), 6.86 (s, 1H), 6.79 (d, J=7.6 Hz, 1H), 6.69 (d, J=8.0 Hz, 1H), 5.34-5.29 (m, 1H), 4.89 (s, 2H), 4.38 (d, J=13.6 Hz, 1H), 4.27 (d, J=13.6 Hz, 1H), 3.42 (d, J=1.2 Hz, 2H), 3.34 (d, J=0.8 Hz, 3H), 3.15-3.11 (m, 1H), 2.86 (d, J=13.6 Hz, 1H), 2.66 (s, 1H), 2.63-2.60 (m, 1H), 2.59 (s, 1H), 2.12-1.98 (m, 4H), 1.96-1.65 (m, 6H), 1.46 (s, 4H), 1.38 (s, 5H), 1.25-1.22 (m, 2H).

### Example 180

### 3-(carboxymethoxy)-4-chloro-5-[3-[[(4S)-1-[3-[4-[1-(2,6-dioxo-3-piperidyl)-2-oxo-benzo[cd]indol-6-yl]-3,3-difluoro-piperidine-1-carbonyl]amino]phenyl]methylsulfonyl]-2,2-dimethyl-4-piperidyl]amino]phenyl]thiophene-2-carboxylic acid (180)

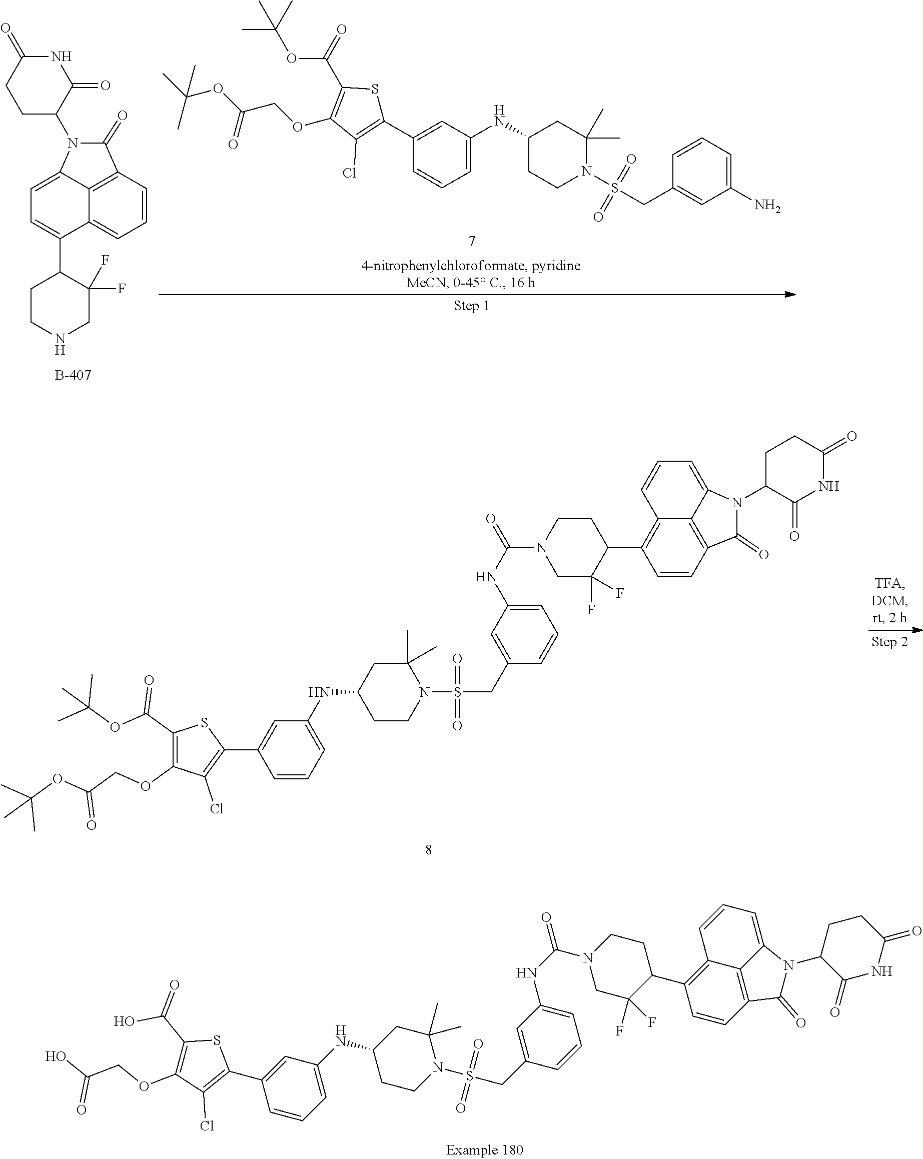 [SMILES: O=C1CCC(N2C(=O)c3cccc4c(C5CCNCC5(F)F)ccc2c34)C(=O)N1 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)N5CCC(c6ccc7c8c(cccc68)N(C6CCC(=O)NC6=O)C7=O)C(F)(F)C5)c4)C(C)(C)C3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)N(C3CCC(=O)NC3=O)C4=O)C(F)(F)C2)c1]

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]-3,3-difluoro-piperidine-1-carbonyl]amino]phenyl]methylsulfonyl]-2,2-dimethyl-4-piperidyl]amino]phenyl]thiophene-2-carboxylate (8)

Into a 50 mL single neck round bottom flask containing a well-stirred solution of tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (7, 400 mg, 544.19 μmol) in MeCN (5 mL) was added pyridine (215.23 mg, 2.72 mmol, 220.07 μL) and 4-nitrophenyl chloroformate (131.62 mg, 653.03 μmol) at 0° C. The mixture was stirred for 2 h at rt. Then, 3-[6-(3,3-difluoro-4-piperidyl)-2-oxo-benzo[cd]indol-1-yl]piperidine-2,6-dione (B-407, 377.56 mg, 544.19 μmol, TFA salt) was added and the reaction mixture was stirred at 45° C. After 16 h, the volatiles were distilled off under reduced pressure to afford tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]-3,3-difluoro-piperidine-1-carbonyl]amino]phenyl]methylsulfonyl]-2,2-dimethyl-4-piperidyl]amino]phenyl]thiophene-2-carboxylate (8, 700 mg, 231.56 μmol, 43% yield, 38% purity) as a pale yellow gummy solid. The material was used in the next step without purification.

LCMS (ES+): m/z 1145.2 [M+H]⁺

### Step 2: 3-(carboxymethoxy)-4-chloro-5-[3-[[(4S)-1-[[3-[[4-[1-(2,6-dioxo-3-piperidyl)-2-oxo-benzo[cd]indol-6-yl]-3,3-difluoro-piperidine-1-carbonyl]amino]phenyl]methylsulfonyl]-2,2-dimethyl-4-piperidyl]amino]phenyl]thiophene-2-carboxylic acid (180)

3-(carboxymethoxy)-4-chloro-5-[3-[[(4S)-1-[[3-[[4-[1-(2,6-dioxo-3-piperidyl)-2-oxo-benzo[cd]indol-6-yl]-3,3-difluoro-piperidine-1-carbonyl]amino]phenyl]methylsulfonyl]-2,2-dimethyl-4-piperidyl]amino]phenyl]thiophene-2-carboxylic acid (180, 87 mg, 81.63 μmol, 39% yield) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]-3,3-difluoro-piperidine-1-carbonyl]amino]phenyl]methylsulfonyl]-2,2-dimethyl-4-piperidyl]amino]phenyl]thiophene-2-carboxylate (8, 700 mg, 211.15 μmol) in a similar fashion to Compound A-26. The residue was purified by reverse phase prep HPLC [Purification method: Column: X-select, C18 (150×19) mm, 5 micron; Mobile phase A: 10 mM ammonium acetate in water; Mobile phase B: MeCN].

LCMS (ES+): m/z 1033.0 [M+H]⁺

¹HNMR (400 MHz, DMSO-d6): δ 11.16 (s, 1H), 8.85 (s, 1H), 8.52 (d, J=8.00 Hz, 1H), 8.14 (d, J=6.80 Hz, 1H), 7.93-7.89 (m, 1H), 7.59-7.50 (m, 2H), 7.30-7.26 (m, 1H), 7.22-7.09 (m, 2H), 7.04-6.96 (m, 2H), 6.83 (s, 1H), 6.77 (d, J=7.60 Hz, 1H), 6.66 (d, J=7.60 Hz, 1H), 5.66 (t, J=7.60 Hz, 1H), 5.49-5.46 (m, 1H), 4.65-4.55 (m, 3H), 4.44-4.26 (m, 4H), 3.02-2.86 (m, 3H), 2.78-2.68 (m, 2H), 2.33-2.29 (m, 1H), 2.12-2.08 (m, 2H), 1.97-1.76 (m, 4H), 1.50-1.41 (m, 7H), 1.20-1.05 (m, 1H).

### Example 181

### 3-(carboxymethoxy)-4-chloro-5-[3-[[(4S)-1-[[3-[[4-[1-(2,6-dioxo-3-piperidyl)-3-isopropyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylic acid (181)

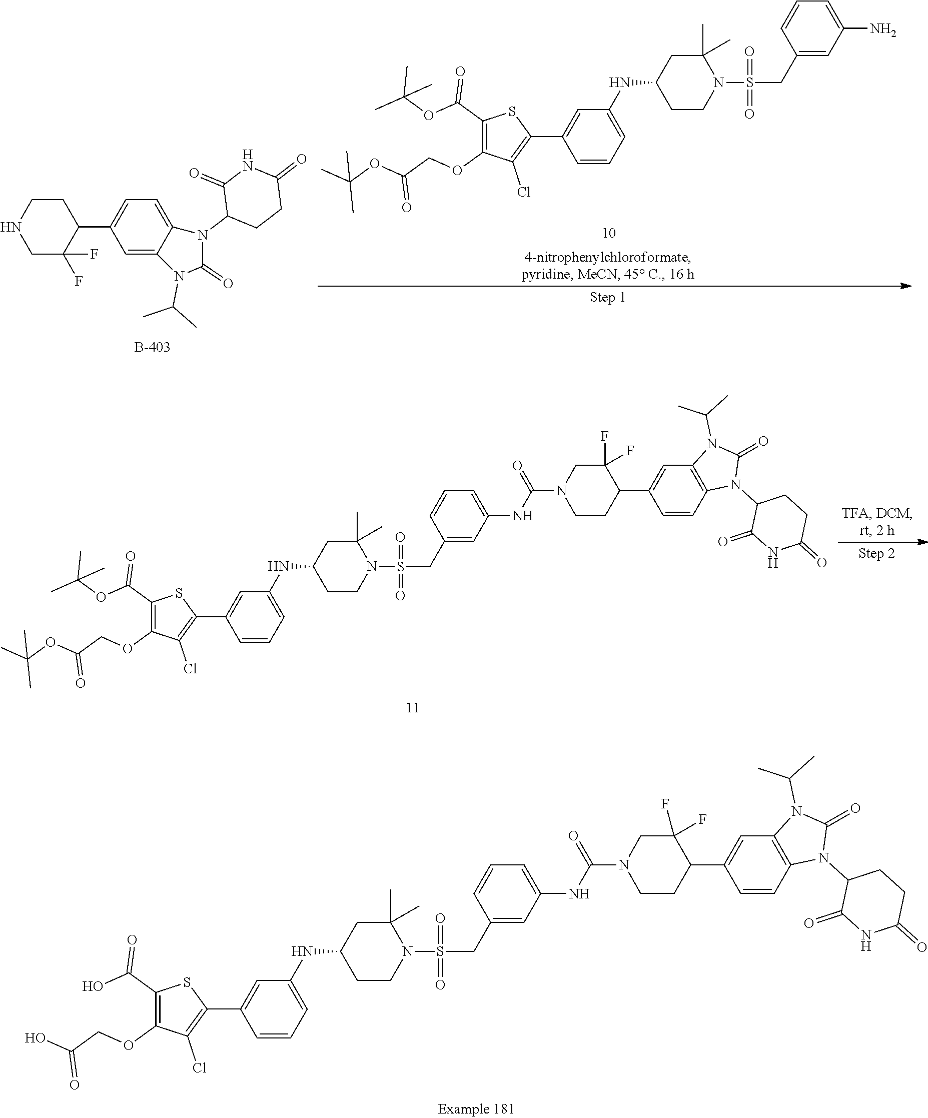

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-3-isopropyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylate (11)

tert-butyl 3-(2-tert-butoxy-2-oxo-ethoxy)-4-chloro-5-[3-[[(4S)-1-[[3-[[4-[1-(2,6-dioxo-3-piperidyl)-3-isopropyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylate (11, 350 mg, 94.97 μmol, 40% yield, 31% purity) was synthesized from tert-butyl5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (10, 170 mg, 236.00 μmol) in a similar fashion to Example 180. The material was taken to next step without purification.

LCMS (ES+): m/z 1153.4 [M+H]⁺

### Step 2: 3-(carboxymethoxy)-4-chloro-5-[3-[[(4S)-1-[[3-[[4-[1-(2,6-dioxo-3-piperidyl)-3-isopropyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylic acid (181)

3-(carboxymethoxy)-4-chloro-5-[3-[[(4S)-1-[[3-[[4-[1-(2,6-dioxo-3-piperidyl)-3-isopropyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylic acid (181, 83 mg, 71.51 μmol, 76% yield, TFA salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-3-isopropyl-2-oxo-benzimidazol-5-yl]-3,3-difluoro-piperidine-1-carbonyl]amino]phenyl]methylsulfonyl]-2,2-dimethyl-4-piperidyl]amino]phenyl]thiophene-2-carboxylate (11, 350 mg, 94.12 μmol) in a similar fashion to Compound A-26. The residue was purified by reverse phase prep HPLC [Purification method: Column: X-Bridge, C18 (150×19) mm, 5 micron; Mobile phase A: 0.1% TFA in water and Mobile phase B: MeCN]. LCMS (ES+): m/z 1040.7 [M+H]⁺

¹HNMR (400 MHz, DMSO-d6): δ 11.11 (s, 1H), 8.82 (s, 1H), 7.58 (s, 1H), 7.50 (d, J=8.00 Hz, 1H), 7.30-7.18 (m, 3H), 7.09-6.97 (m, 3H), 6.88 (s, 1H), 6.81 (d, J=8.00 Hz, 1H), 6.70 (d, J=8.40 Hz, 1H), 5.37-5.33 (m, 1H), 4.92 (s, 2H), 4.64-4.59 (m, 2H), 4.40-4.26 (m, 3H), 3.48-3.33 (m, 3H), 3.25-3.05 (m, 3H), 3.02-2.90 (m, 1H), 2.72-2.60 (m, 2H), 2.09-2.08 (m, 1H), 2.03-2.00 (m, 1H), 1.88-1.78 (m, 3H), 1.50-1.40 (m, 13H), 1.12-1.05 (m, 1H).

### Example 182

### 3-(carboxymethoxy)-4-chloro-5-(3-(((4S)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylic acid (182)

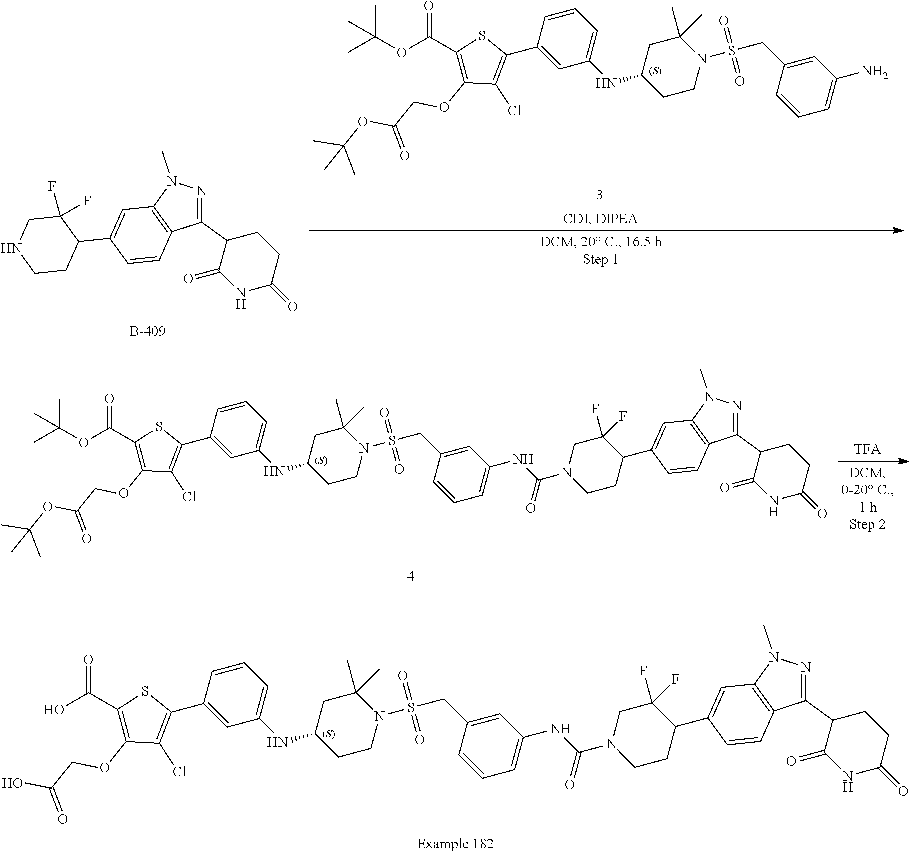

### Step 1: tert-butyl 3-(2-(tert-butoxy)-2-oxoethoxy)-4-chloro-5-(3-(((4S)-1-((3-(4-(3-(2,6-dioxopiperidin-3-yl)-1-methyl-1H-indazol-6-yl)-3,3-difluoropiperidine-1carboxamido)benz yl)sulfonyl)-2,2-dimethylpiperidin-4-yl)amino)phenyl)thiophene-2-carboxylate (4)

tert-butyl 3-(2-(tert-butoxy)-2-oxoethoxy)-4-chloro-5-(3-(((4S)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylate (4, 100 mg, 90.20 μmol, 54% yield) was synthesized from(S)-tert-butyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (3, 120 mg, 166.59 μmol) and 3-(6-(3,3-difluoropiperidin-4-yl)-1-methyl-1H-indazol-3-yl)piperidine-2,6-dione (B-409, 60.37 mg, 166.59 μmol, HCl salt) in a similar fashion to Compound B-242. The residue was purified by prep-TLC (SiO2, EtOAc, Rf=0.4). LCMS (ESI): m/z 1108.6 [M+H]⁺

### Step 2: 3-(carboxymethoxy)-4-chloro-5-(3-(((4S)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylic acid (182)

3-(carboxymethoxy)-4-chloro-5-(3-(((4S)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylic acid (182, 55.22 mg, 49.72 μmol, TFA salt) was synthesized from tert-butyl 3-(2-(tert-butoxy)-2-oxoethoxy)-4-chloro-5-(3-(((4S)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylate (4, 97 mg, 87.49 μmol) in a similar fashion to Compound A-26. The residue was purified by prep-HPLC (flow: 25 mL/min; gradient: from 32-62% MeCN in water (0.1% TFA) over 10 min; column: Phenomenex Luna C18 150×25 mm×10 μm). LCMS (ESI): m/z 996.7 (M+H)⁺.

¹H NMR (400 MHz, DMSO-d₆) δ 10.88 (s, 1H), 8.81 (s, 1H), 7.66 (d, J=8.4 Hz, 1H), 7.56 (d, J=12.0 Hz, 2H), 7.49 (d, J=8.6 Hz, 1H), 7.29-7.23 (m, 1H), 7.19 (t, J=8.0 Hz, 1H), 7.11-7.07 (m, 1H), 7.04-6.97 (m, 1H), 6.87 (s, 1H), 6.80 (d, J=8.0 Hz, 1H), 6.69 (d, J=8.0 Hz, 1H), 4.90 (s, 2H), 4.64-4.58 (m, 1H), 4.40-4.33 (m, 3H), 4.27 (d, J=13.6 Hz, 1H), 3.99 (s, 3H), 3.19-3.04 (m, 6H), 2.67-2.62 (m, 2H), 2.39-2.32 (m, 1H), 2.29-2.21 (m, 1H), 2.22-2.14 (m, 1H), 1.96-1.84 (m, 2H), 1.79 (d, J=12.0 Hz, 1H), 1.50-1.38 (m, 7H), 1.23-1.10 (m, 1H).

### Example 183

### 3-(carboxymethoxy)-4-chloro-5-[3-[[(4S)-1-[[3-[[2-[1-[1-(2,6-dioxo-3-piperidyl)-3-methyl-2-oxo-benzimidazol-5-yl]-4-hydroxy-4-piperidyl]acetyl]amino]phenyl]methylsulfonyl]-2,2-dimethyl-4-piperidyl]amino]phenyl]thiophene-2-carboxylic acid (183)

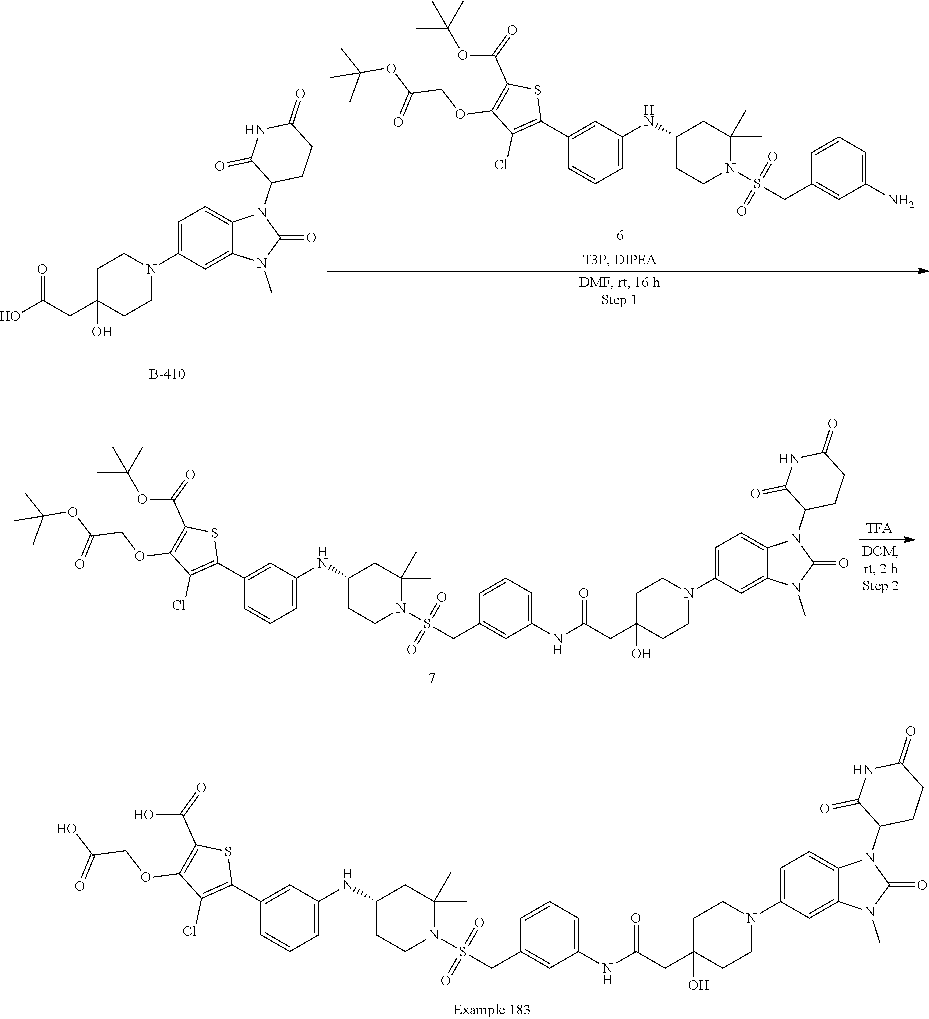

### Step 1: tert-butyl 3-(2-tert-butoxy-2-oxo-ethoxy)-4-chloro-5-[3-[[(4S)-1-[[3-[2-[1-[1-(2,6-dioxo-3-piperidyl)-3-methyl-2-oxo-benzimidazol-5-yl]-4-hydroxy-4-piperidyl]acetyl]amino]phenyl]methylsulfonyl]-2,2-dimethyl-4-piperidyl]amino]phenyl]thiophene-2-carboxylate (7)

tert-butyl 3-(2-tert-butoxy-2-oxo-ethoxy)-4-chloro-5-[3-[[(4S)-1-[3-[2-[1-[[1-(2,6-dioxo-3-piperidyl)-3-methyl-2-oxo-benzimidazol-5-yl]-4-hydroxy-4-piperidyl]acetyl]amino]phenyl]methylsulfonyl]-2,2-dimethyl-4-piperidyl]amino]phenyl]thiophene-2-carboxylate (7, 160 mg, 118.10 μmol, 43% yield) was synthesized from 2-[1-[1-(2,6-dioxo-3-piperidyl)-3-methyl-2-oxo-benzimidazol-5-yl]-4-hydroxy-4-piperidyl]acetic acid (B-410, 115.62 mg, 217.97 μmol, TFA salt) and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (6, 200 mg, 277.65 μmol) in a similar fashion to Compound A-88. The residue was purified by silica gel flash chromatography using (230-400 mesh silica gel, 0-5% Methanol in DCM).

LCMS (ES+): m/z 1118.3 [M+H]⁺

### Step 2: 3-(carboxymethoxy)-4-chloro-5-[3-[[(4S)-1-[[3-[2-[1-[1-(2,6-dioxo-3-piperidyl)-3-methyl-2-oxo-benzimidazol-5-yl]-4-hydroxy-4-piperidyl]acetyl]amino]phenyl]methylsulfonyl]-2,2-dimethyl-4-piperidyl]amino]phenyl]thiophene-2-carboxylic acid (183)

3-(carboxymethoxy)-4-chloro-5-[3-[[(4S)-1-[3-[2-[1-[1-(2,6-dioxo-3-piperidyl)-3-methyl-2-oxo-benzimidazol-5-yl]-4-hydroxy-4-piperidyl]acetyl]amino]phenyl]methylsulfonyl]-2,2-dimethyl-4-piperidyl]amino]phenyl]thiophene-2-carboxylic acid (183, 71 mg, 68.86 μmol, 48% yield) was synthesized from tert-butyl 3-(2-tert-butoxy-2-oxo-ethoxy)-4-chloro-5-[3-[[(4S)-1-[[3-[[2-[1-[1-(2,6-dioxo-3-piperidyl)-3-methyl-2-oxo-benzimidazol-5-yl]-4-hydroxy-4-piperidyl]acetyl]amino]phenyl]methylsulfonyl]-2,2-dimethyl-4-piperidyl]amino]phenyl]thiophene-2-carboxy late (7, 160 mg, 143.02 μmol) in a similar fashion to Compound A-26. The residue was subjected to purification by a reverse-phase preparative HPLC [Column: X Select C18 (250×19) mm, 5 μm; Mobile phase A: 0.1% ammonium acetate in water and Mobile phase B: CH₃CN].

¹H-NMR (400 MHz, DMSO-d6): δ 11.06 (s, 1H), 9.97 (s, 1H), 7.73 (s, 1H), 7.60 (d, J=8.80 Hz, 1H), 7.31 (t, J=7.60 Hz, 1H), 7.18 (t, J=7.60 Hz, 1H), 7.09 (d, J=8.00 Hz, 1H), 6.95-6.78 (m, 4H), 6.71-6.59 (m, 2H), 5.70-5.61 (m, 1H), 5.81-5.73 (m, 1H), 4.79-4.75 (m, 2H), 4.43-4.28 (m, 2H), 3.44-3.40 (m, 2H), 3.33-3.08 (m, 8H), 2.98-2.76 (m, 1H), 2.70-2.55 (m, 1H), 2.08-1.70 (m, 9H), 1.47-1.40 (m, 7H), 1.20-1.08 (m, 1H). LCMS (ES+): m/z 1006.3 [M+H]⁺.

### Example 184

### 3-(carboxymethoxy)-4-chloro-5-[3-[[(4S)-1-[[3-[[2-[3-[[1-(2,6-dioxo-3-piperidyl)-3-methyl-2-oxo-benzimidazol-5-yl]azetidin-1-yl]acetyl]amino]phenyl]methylsulfonyl]-2,2-dimethyl-4-piperidyl]amino]phenyl]thiophene-2-carboxylic acid (184)

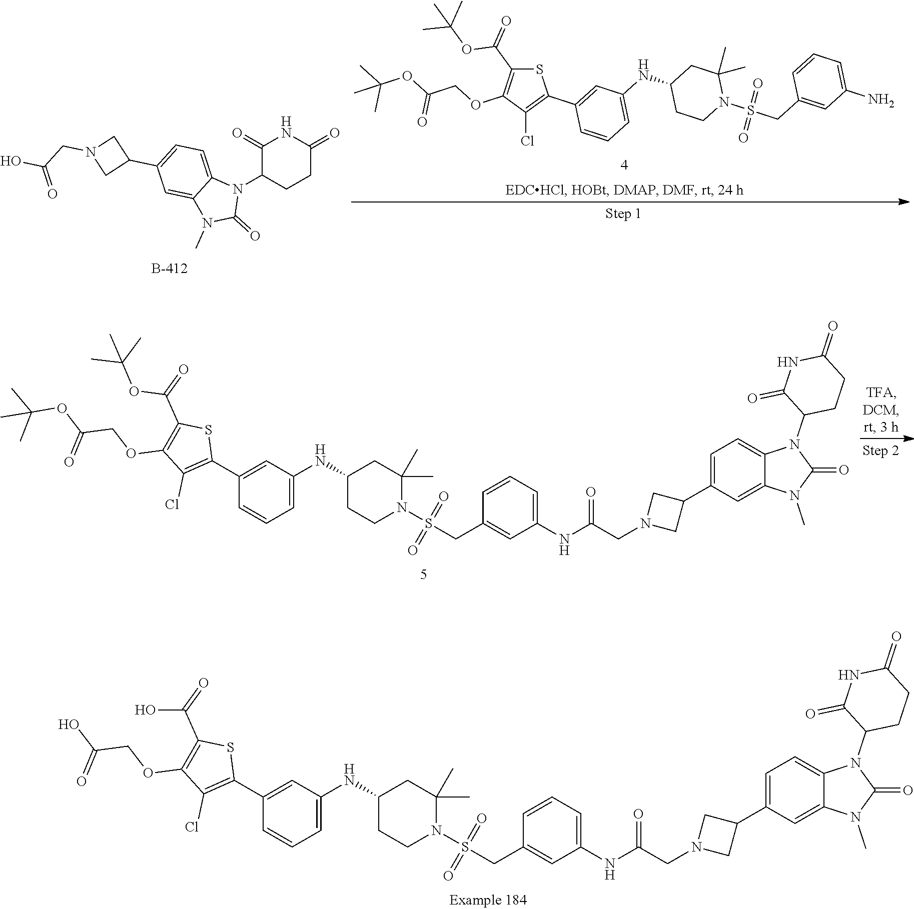 [SMILES: Cn1c(=O)n(C2CCC(=O)NC2=O)c2ccc(C3CN(CC(=O)O)C3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(C3CN(CC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)C3)cc21 ; Cn1c(=O)n(C2CCC(=O)NC2=O)c2ccc(C3CN(CC(=O)Nc4cccc(CS(=O)(=O)N5CC[C@H](Nc6cccc(-c7sc(C(=O)O)c(OCC(=O)O)c7Cl)c6)CC5(C)C)c4)C3)cc21]

### Step 1: tert-butyl 3-(2-tert-butoxy-2-oxo-ethoxy)-4-chloro-5-[3-[[(4S)-1-[[3-[[2-[3-[[1-(2,6-dioxo-3-piperidyl)-3-methyl-2-oxo-benzimidazol-5-yl]azetidin-1-yl]acetyl]amino]phenyl]methylsulfonyl]-2,2-dimethyl-4-piperidyl]amino]phenyl]thiophene-2-carboxylate (5)

tert-butyl 3-(2-tert-butoxy-2-oxo-ethoxy)-4-chloro-5-[3-[[(4S)-1-[[3-[[2-[3-[1-(2,6-dioxo-3-piperidyl)-3-methyl-2-oxo-benzimidazol-5-yl]azetidin-1-yl]acetyl]amino]phenyl]methylsulfonyl]-2,2-dimethyl-4-piperidyl]amino]phenyl]thiophene-2-carboxylate (5, 180 mg, 141.86 μmol, 58% yield, HCl salt) was synthesized from 2-[3-[[1-(2,6-dioxo-3-piperidyl)-3-methyl-2-oxo-benzimidazol-5-yl]azetidin-1-yl]acetic acid (B-412, 110 mg, 244.50 μmol, Formic acid salt) and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (4, 176.12 mg, 244.50 μmol) in a similar fashion to Example 166, except with the addition of Excess HOBt. The solid was filtered and dried under reduced pressure.

LCMS (ES+): m/z 1074.4 [M+H]⁺

### Step 2: 3-(carboxymethoxy)-4-chloro-5-[3-[[(4S)-1-[[3-[[2-[3-[[1-(2,6-dioxo-3-piperidyl)-3-methyl-2-oxo-benzimidazol-5-yl]azetidin-1-yl]acetyl]amino]phenyl]methylsulfonyl]-2,2-dimethyl-4-piperidyl]amino]phenyl]thiophene-2-carboxylic acid (184)

3-(carboxymethoxy)-4-chloro-5-[3-[[(4S)-1-[[3-[[2-[3-[[1-(2,6-dioxo-3-piperidyl)-3-methyl-2-oxo-benzimidazol-5-yl]azetidin-1-yl]acetyl]amino]phenyl]methylsulfonyl]-2,2-dimethyl-4-piperidyl]amino]phenyl]thiophene-2-carboxylic acid (184, 115 mg, 104.53 μmol, 74% yield, TFA salt) was synthesized from tert-butyl 3-(2-tert-butoxy-2-oxo-ethoxy)-4-chloro-5-[3-[[(4S)-1-[[3-[[2-[3-[[1-(2,6-dioxo-3-piperidyl)-3-methyl-2-oxo-benzimidazol-5-yl]azetidin-1-yl]acetyl]amino]phenyl]methylsulfonyl]-2,2-dimethyl-4-piperidyl]amino]phenyl]thiophene-2-carboxylate (5, 180 mg, 140.93 μmol, HCl salt) in a similar fashion to Compound A-26. The residue was purified by reverse phase prep HPLC [Purification method: Column: YMC C18 (250×20) mm, 5 micron; Mobile phase: 0.1% TFA in water; Mobile phase B: MeCN].

LCMS (ES+): m/z 962.2 [M+H]⁺

¹HNMR (400 MHz, DMSO-d6): δ 11.11 (s, 1H), 10.65 (s, 1H), 10.01 (s, 1H), 7.71 (s, 1H), 7.57 (d, J=8.00 Hz, 1H), 7.40-7.35 (m, 2H), 7.18-7.07 (m, 4H), 6.87 (s, 1H), 6.80 (d, J=7.60 Hz, 1H), 6.67 (d, J=8.00 Hz, 1H), 5.39-5.34 (m, 1H), 4.91 (s, 2H), 4.53-4.20 (m, 8H), 3.54-3.42 (m, 4H), 3.37 (s, 3H), 3.17-3.13 (m, 1H), 2.90-2.81 (m, 1H), 2.73-2.62 (m, 2H), 2.03-2.01 (m, 1H), 1.90-1.78 (m, 2H), 1.46 (s, 3H), 1.39 (s, 3H), 1.13-1.10 (m, 1H).

### Example 185

### 3-(carboxymethoxy)-4-chloro-5-(3-(((4R)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylic acid (185)

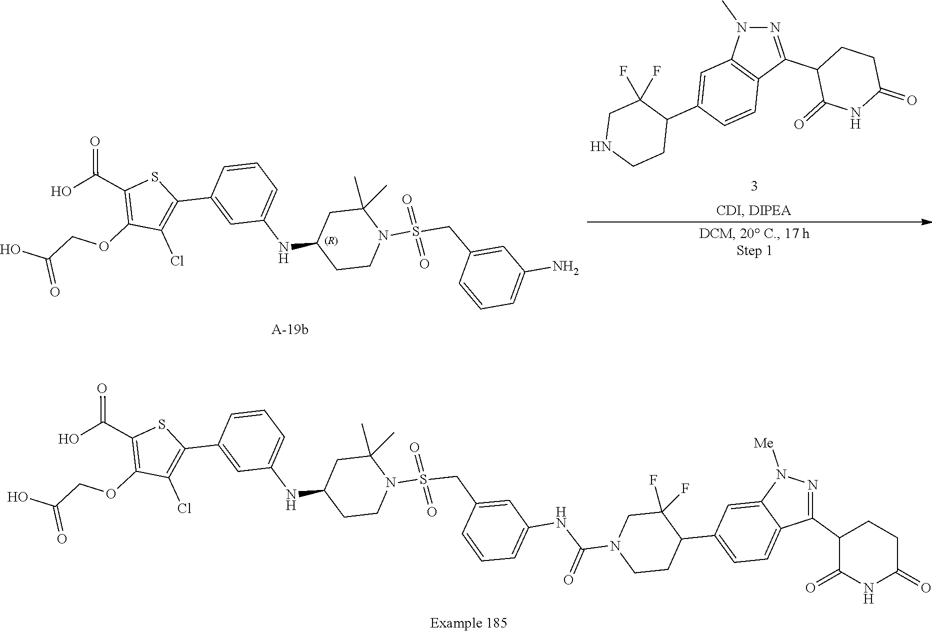

### Step 1: 3-(carboxymethoxy)-4-chloro-5-(3-(((4R)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylic acid (185)

3-(carboxymethoxy)-4-chloro-5-(3-(((4R)-1-((3-(4-(3-(2,6-dioxopiperidin-3-yl)-1-methyl-1H-indazol-6-yl)-3,3-difluoropiperidine-1-carboxamido)benzyl)sulfonyl)-2,2-dimethylpiperidin-4-yl)amino)phenyl)thiophene-2-carboxylic acid (185, 29 mg, 26.11 μmol, 16% yield, TFA salt) was synthesized from (R)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19b, 100 mg, 164.44 μmol) in DCM (2 mL) and 3-[6-(3,3-difluoro-4-piperidyl)-1-methyl-indazol-3-yl]piperidine-2,6-dione (B-409, 65.58 mg, 164.44 μmol, HCl salt) in a similar fashion to Compound B-242. The residue was purified by reversed phase flash chromatography (flow: 27 mL/min; gradient: from 36-66% water (0.1% TFA) in MeCN over 10 min; column: Phenomenex Synergi C18 150×25 mm×10 μm).

LCMS (ESI): m/z 996.0 (M+H)⁺.

¹H NMR (400 MHz, DMSO-d6) δ 10.90 (s, 1H), 8.82 (s, 1H), 7.67 (d, J=8.4 Hz, 1H), 7.58 (d, J=10.8 Hz, 2H), 7.51 (d, J=7.8 Hz, 1H), 7.28 (t, J=7.8 Hz, 1H), 7.20 (t, J=7.8 Hz, 1H), 7.09 (d, J=8.4 Hz, 1H), 7.03 (d, J=7.6 Hz, 1H), 6.88 (s, 1H), 6.81 (d, J=7.8 Hz, 1H), 6.70 (d, J=8.0 Hz, 1H), 4.91 (s, 2H), 4.70-4.56 (m, 1H), 4.44-4.33 (m, 3H), 4.28 (d, J=13.8 Hz, 1H), 4.00 (s, 3H), 3.35-3.29 (m, 3H), 3.21-3.03 (m, 3H), 2.70-2.56 (m, 2H), 2.44-2.35 (m, 1H), 2.31-2.12 (m, 2H), 2.00-1.74 (m, 3H), 1.55-1.36 (m, 7H), 1.20-1.04 (m, 1H).

### Example 186

### 5-(3-(((4S)-1-((3-(4-(3-(tert-butyl)-1-(2,6-dioxopiperidin-3-yl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)-3-(carboxymethoxy)-4-chlorothiophene-2-carboxylic acid (186)

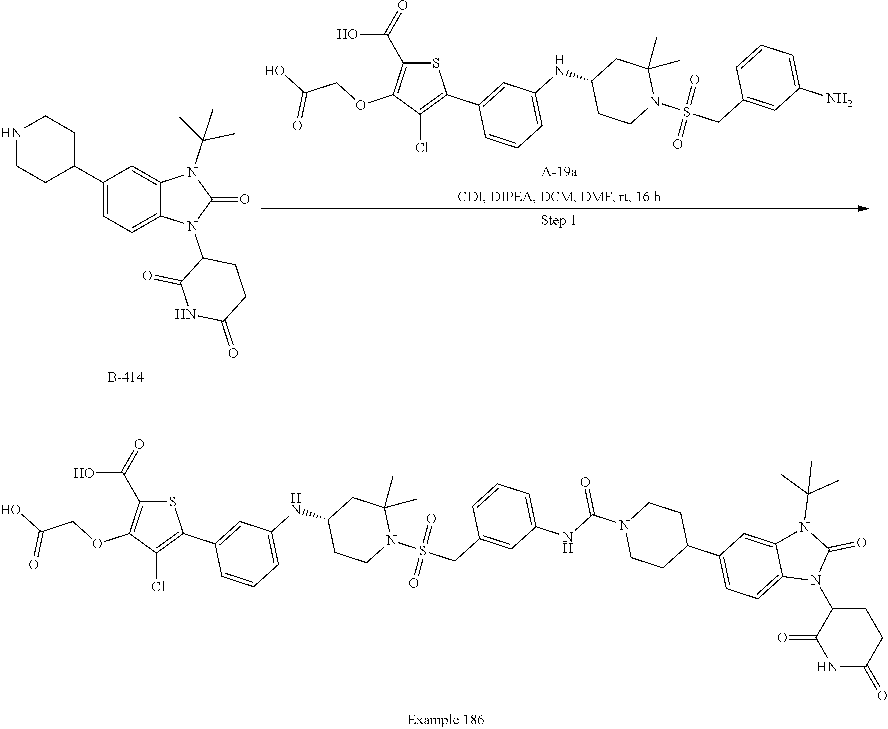 [SMILES: CC(C)(C)n1c(=O)n(C2CCC(=O)NC2=O)c2ccc(C3CCNCC3)cc21 ; CC1(C)C[C@@H](Nc2cccc(-c3sc(C(=O)O)c(OCC(=O)O)c3Cl)c2)CCN1S(=O)(=O)Cc1cccc(N)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C(C)(C)C)c(=O)n4C3CCC(=O)NC3=O)CC2)c1]

### Step 1:5-[3-[[(4S)-1-[[3-[[4-[3-tert-butyl-1-(2,6-dioxo-3-piperidyl)-2-oxo-benzimidazol-5-yl]piperidine-1-carbonyl]amino]phenyl]methylsulfonyl]-2,2-dimethyl-4-piperidyl]amino]phenyl]-3-(carboxymethoxy)-4-chloro-thiophene-2-carboxylic acid (186)

5-[3-[[(4S)-1-[[3-[[4-[3-tert-butyl-1-(2,6-dioxo-3-piperidyl)-2-oxo-benzimidazol-5-yl]piperidine-1-carbonyl]amino]phenyl]methylsulfonyl]-2,2-dimethyl-4-piperidyl]amino]phenyl]-3-(carboxymethoxy)-4-chloro-thiophene-2-carboxylic acid (186, 20 mg, 17.61 μmol, 23% yield, TFA salt) was synthesized from 5-[3-[[(4S)-1-[(3-aminophenyl)methylsulfonyl]-2,2-dimethyl-4-piperidyl]amino]phenyl]-3-(carboxymethoxy)-4-chloro-thiophene-2-carboxylic acid (A-19a, 60 mg, 77.27 μmol, TFA salt) and 3-[3-tert-butyl-2-oxo-5-(4-piperidyl)benzimidazol-1-yl]piperidine-2,6-dione (B-414, 40.06 mg, 77.27 μmol, TFA salt) in a similar fashion to Compound B-242. The residue was purified by reverse phase column chromatography [Purification method: Silicycle C18 column; Mobile phase A: 0.1% TFA in water; Mobile phase B: MeCN.

LCMS (ES+): m/z 1018.0 [M+H]⁺

¹HNMR (400 MHz, DMSO-d6): δ 11.07 (s, 1H), 8.62 (s, 1H), 7.57 (s, 1H), 7.47 (d, J=9.20 Hz, 1H), 7.35 (s, 1H), 7.27-7.18 (m, 2H), 7.00-6.98 (m, 2H), 6.91 (d, J=8.00 Hz, 1H), 6.87 (s, 1H), 6.81 (d, J=8.40 Hz, 1H), 6.70 (d, J=8.00 Hz, 1H), 5.31-5.28 (m, 1H), 4.91 (s, 2H), 4.38-4.24 (m, 4H), 3.28-3.10 (m, 1H), 2.91-2.71 (m, 3H), 2.68-2.65 (m, 2H), 1.90-1.77 (m, 6H), 1.76 (s, 9H), 1.72-1.62 (m, 2H), 1.59-1.43 (m, 8H), 1.15-1.05 (m, 1H).

### Example 187a and Example 187b

### 3-(carboxymethoxy)-4-chloro-5-[3-[[(4S)-1-[[3-[[4-[1-[(3R)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (187a) and 3-(carboxymethoxy)-4-chloro-5-[3-[[(4S)-1-[3-[4-[1-[(3S)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (187b)

Configurations are arbitrarily assigned.

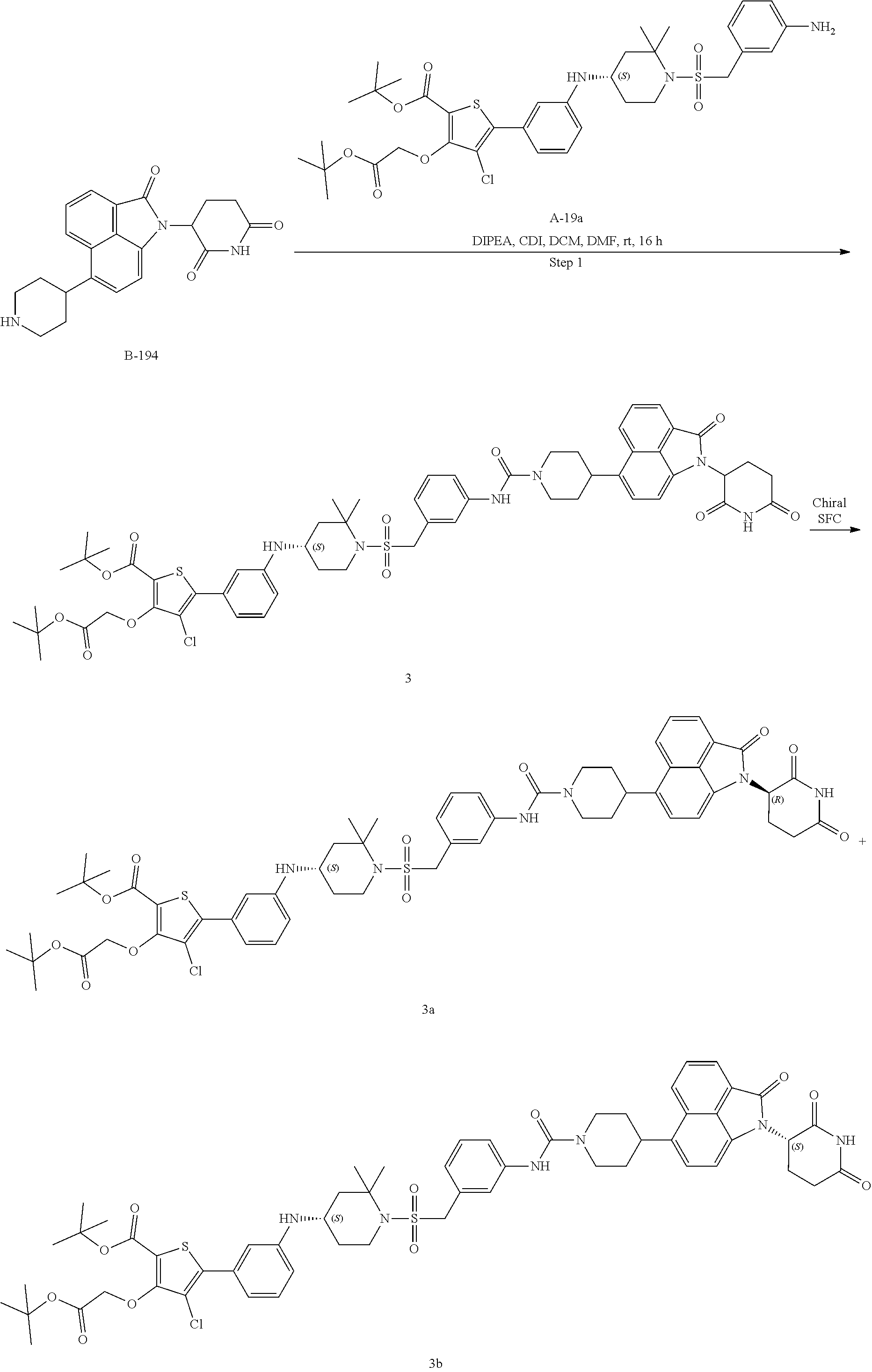

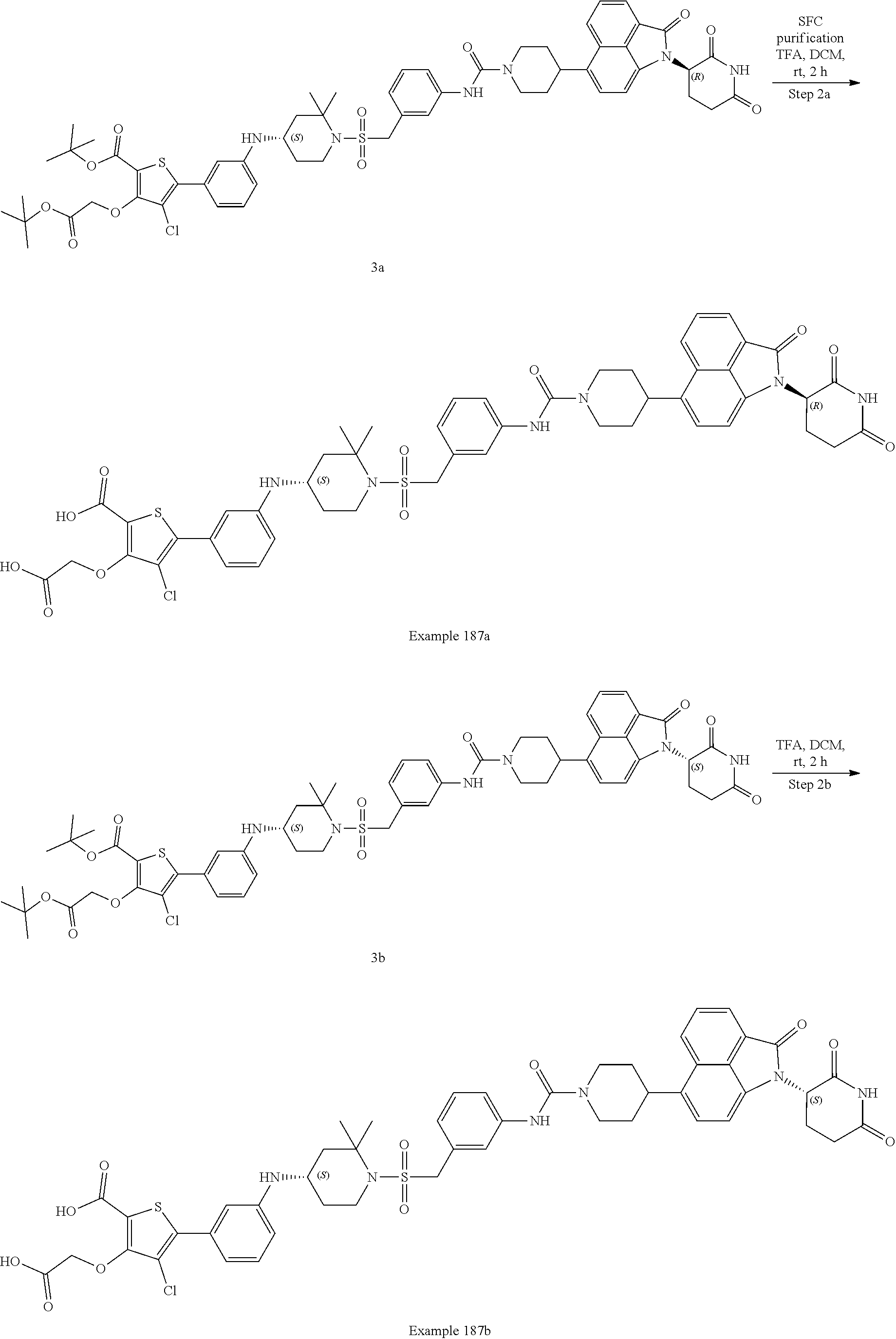

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylate (3)

Into a 25 mL single neck round bottom flask containing a well-stirred solution of tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (A-19a, 400 mg, 544.19 μmol) in DCM (5 mL) and DMF (0.5 mL) were added N,N-diisopropyletbylamine (703.31 mg, 5.44 mmol, 947.86 μL) and 1,1′-carbonyldiimidazole (141.18 mg, 870.70 μmol) at rt and stirred for 2 h. Then, a solution of 3-[2-oxo-6-(4-piperidyl)benzo[cd]indol-1-yl]piperidine-2,6-dione (B-194, 355.91 mg, 544.19 μmol, TFA salt) in DMF (2 mL) and DIPEA (0.8 mL) was added. After 16 h, the reaction mixture was concentrated and purified by reverse phase column chromatography [Siliasep C18 120 g; Mobile phase A: 0.1% TFA in water; Mobile phase B: MeCN] to afford tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylate (3, 360 mg, 324.40 μmol, 60% yield) as a pale yellow solid.

LCMS (ES+): m/z 1109.2 [M+H]⁺

The Diastercomers 3a and 3b were separated by chiral SFC:
Instrument: PIC 175
Column: (R,R) Whelk-01 (250*30) mm, 5p
Mobile Phase: {(CO₂: 0.2% TFA in IPA:MeCN (1:1) (60:40)}
Total flow: 100 g/min
Back pressure: 100 bar
Wavelength: 210 nm
Cycle time: 11.50 min 360 mg of sample (3) is dissolved in 6 mL of Acetonitrile and injected 0.5 mL per injection.

First eluted fraction—Diastereomer 1 (3a): 1-tert-butyl 3-(2-tert-butoxy-2-oxo-ethoxy)-4-chloro-5-[3-[[(4S)-1-[[3-[[4-[1-[(3R)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylate (3a, 150 mg, 125.16 μmol, 23% yield, TFA salt) (RT 3.82 min, Chiral purity 92.22%) as a pale yellow gummy solid.

LCMS (ES+): m/z 1109.2 [M+H]⁺

Late eluted fraction—Diastereomer 2 (3b): tert-butyl 3-(2-tert-butoxy-2-oxo-ethoxy)-4-chloro-5-[3-[(4S)-1-[[3-[[4-[1-[(3S)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylate (3b, 160 mg, 126.56 μmol, 23% yield) (RT 5.71 min, chiral purity 93.83%) as a pale yellow gummy solid. LCMS (ES+): m/z 1109.2 [M+H]⁺

### Step 2a: 3-(carboxymethoxy)-4-chloro-5-[3-[[(4S)-1-[3-[4-[1-[(3R)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (187a)

Into a 50 mL single neck round bottomed flask containing a well-stirred solution of tert-butyl 3-(2-tert-butoxy-2-oxo-ethoxy)-4-chloro-5-[3-[[(4S)-1-[[3-[[4-[[1-[(3R)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylate (3a, 200.00 mg, 160.16 μmol, TFA salt) in DCM (4 mL) was added trifluoroacetic acid (182.62 mg, 1.60 mmol, 123.39 μL). After 2 h, the reaction mixture was concentrated and purified by reverse phase prep HPLC [Purification method: Column: XSelect C18 (250×19) mm 5 microns; Mobile phase A: 0.1% TFA in water; Mobile phase B: MeCN] to afford 3-(carboxymethoxy)-4-chloro-5-[3-[[(4S)-1-[[3-[[4-[1-[(3R)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (187a, 133 mg, 117.44 μmol, 73% yield, TFA salt) as an off-white solid. LCMS (ES−): m/z 995.0 [M−H]⁻

¹HNMR (400 MHz, DMSO-d6): δ 13.33 (bs, 1H), 11.12 (s, 1H), 8.66 (s, 1H), 8.50 (d, J=8.40 Hz, 1H), 8.12 (d, J=7.20 Hz, 1H), 7.90-7.86 (m, 1H), 7.59 (s, 1H), 7.49 (d, J=8.40 Hz, 1H), 7.39 (d, J=7.60 Hz, 1H), 7.28-7.18 (m, 2H), 7.10 (d, J=7.60 Hz, 1H), 7.00 (d, J=7.60 Hz, 1H), 6.88 (s, 1H), 6.81 (d, J=7.60 Hz, 1H), 6.70 (d, J=8.00 Hz, 1H), 5.46-5.43 (m, 1H), 4.91 (s, 2H), 4.39-4.25 (m, 4H), 3.61-3.45 (m, 3H), 3.13-2.96 (m, 4H), 2.78-2.64 (m, 2H), 2.11-2.08 (m, 1H), 1.93-1.71 (m, 5H), 1.50-1.40 (m, 7H), 1.15-1.04 (m, 1H).

### Step 2b: 3-(carboxymethoxy)-4-chloro-5-[3-[[(4S)-1-[3-[4-[1-[(3S)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (187b)

3-(carboxymethoxy)-4-chloro-5-[3-[[(4S)-1-[3-[4-[1-[(3S)-2,6-dioxo-3-piperidyl]-2-oxo-benzo[cd]indol-6-yl]piperidine-1-carbonyl]amino]phenyl]methylsulfonyl]-2,2-dimethyl-4-piperidyl]amino]phenyl]thiophene-2-carboxylic acid (187b, 142 mg, 127.30 μmol, 76% yield, TFA salt) was synthesized following the same procedure as Compound 187a. LCMS (ES−): m/z 995.0 [M−H]⁻

¹HNMR (400 MHz, DMSO-d6): δ 13.25 (bs, 1H), 11.13 (s, 1H), 8.67 (s, 1H), 8.50 (d, J=8.40 Hz, 1H), 8.12 (d, J=7.20 Hz, 1H), 7.90-7.86 (m, 1H), 7.59 (s, 1H), 7.49 (d, J=8.00 Hz, 1H), 7.39 (d, J=7.60 Hz, 1H), 7.28-7.18 (m, 2H), 7.10 (d, J=7.20 Hz, 1H), 7.00 (d, J=7.60 Hz, 1H), 6.88 (s, 1H), 6.81 (d, J=7.20 Hz, 1H), 6.70 (d, J=8.00 Hz, 1H), 5.47-5.42 (m, 1H), 4.91 (s, 2H), 4.39-4.25 (m, 4H), 3.61-3.45 (m, 3H), 3.13-2.96 (m, 4H), 2.78-2.64 (m, 2H), 2.11-2.08 (m, 1H), 1.93-1.68 (m, 6H), 1.50-1.40 (m, 7H), 1.15-1.04 (m, 1H).

### Example 188 and Example 189

### 3-(carboxymethoxy)-4-chloro-5-(3-(((4S)-1-((3-(3-(1-(1-(2,6-dioxopiperidin-3-yl)-2-oxo-1,2-dihydrobenzo[cd]indol-6-yl)piperidin-4-yl)-2-oxoimidazolidin-1-yl)benzyl)sulfonyl)-2,2-dimethylpiperidin-4-yl)amino)phenyl)thiophene-2-carboxylic acid (189)

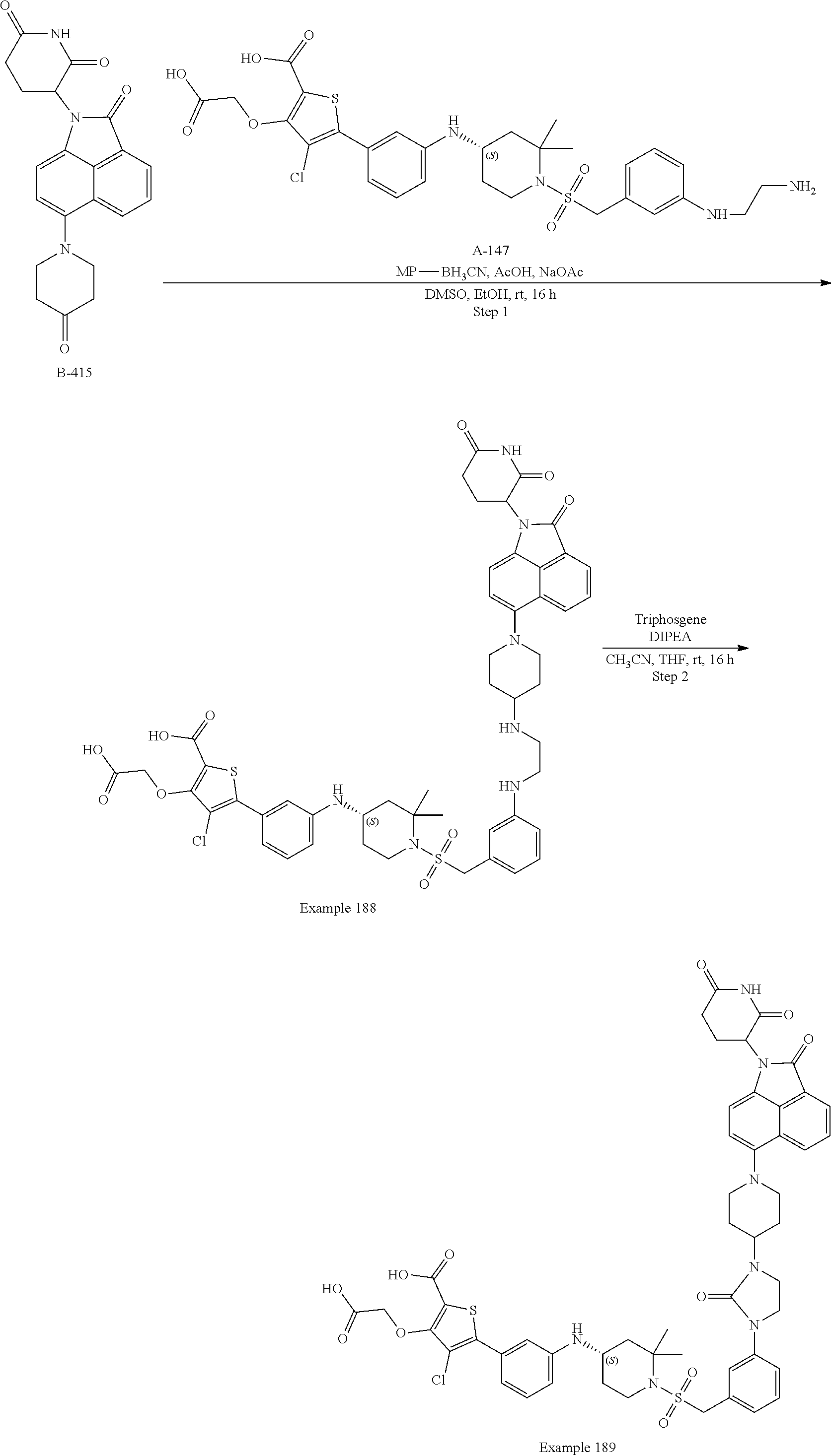

### Step 1: 3-(carboxymethoxy)-4-chloro-5-(3-(((4S)-1-((3-((2-((1-(1-(2,6-dioxopiperidin-3-yl)-2-oxo-1,2-dihydrobenzo[cd]indol-6-yl)piperidin-4-yl)amino)ethyl)amino)benzyl)sulfonyl)-2,2-dimethylpiperidin-4-yl)amino)phenyl)thiophene-2-carboxylic acid (188)

3-(carboxymethoxy)-4-chloro-5-[3-[[(4S)-1-[3-[2-[1-[1-(2,6-dioxo-3-piperidyl)-2-oxo-benzo[cd]indol-6-yl]-4-piperidyl]amino]ethylamino]phenyl]methylsulfonyl]-2,2-dimethyl-4-piperidyl]amino]phenyl]thiophene-2-carboxylic acid (188, 100 mg, 92.19 μmol, 46% yield, formic acid salt) was synthesized from 3-[2-oxo-6-(4-oxo-1-piperidyl)benzo[cd]indol-1-yl]piperidine-2,6-dione (B-415, 91.09 mg, 198.79 μmol, formic acid salt) and 5-[3-[[(4S)-1-[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147, 140 mg, 198.79 μmol, formic acid salt) in a similar fashion to Compound A-74a/b, except that DMSO/EtOH was used as the solvent and 1 eq. sodium acetate was included. The residue was purified by reverse-phase column chromatography [Column: REDISEP-C18-120 g; Mobile Phase A: 0.1% TFA in water and Mobile Phase B: MeCN].

1H-NMR (400 MHz, DMSO-d6): δ 11.12 (s, 1H), 8.23 (s, 1H), 8.22 (d, J=8.40 Hz, 1H), 8.10 (d, J=6.80 Hz, 1H), 7.85 (t, J=1.20 Hz, 1H), 7.19-7.15 (m, 2H), 7.02 (d, J=7.60 Hz, 1H), 6.96 (d, J=8.00 Hz, 1H), 6.81-6.77 (m, 2H), 6.71-6.56 (m, 4H), 5.89 (s, 1H), 5.71 (d, J=7.60 Hz, 1H), 5.44 (dd, J=5.20, 12.80 Hz, 1H), 4.62 (d, J=Hz, 1H), 4.34-4.21 (m, 2H), 3.51-3.31 (m, 7H), 3.01-2.90 (m, 2H), 2.85-2.55 (m, 4H), 2.19-2.09 (m, 4H), 1.92-1.78 (m, 5H), 1.49-1.36 (m, 7H), 1.24-1.08 (m, 1H). LCMS (ES+): m/z 1012.0 [M+H]⁺

### Step 2: 3-(carboxymethoxy)-4-chloro-5-(3-(((4S)-1-((3-(3-(1-(1-(2,6-dioxopiperidin-3-yl)-2-oxo-1,2-dihydrobenzo[cd]indol-6-yl)piperidin-4-yl)-2-oxoimidazolidin-1-yl)benzyl)sulfonyl)-2,2-dimethylpiperidin-4-yl)amino)phenyl)thiophene-2-carboxylic acid (189)

Into a 8 mL screw-capped vial containing a well-stirred solution of 3-(carboxymethoxy)-4-chloro-5-[3-[[(4S)-1-[3-[2-[[1-[1-(2,6-dioxo-3-piperidyl)-2-oxo-benzo[cd]indol-6-yl]-4-piperidyl]amino]ethylamino]phenyl]methylsulfonyl]-2,2-dimethyl-4-piperidyl]amino]phenyl]thiophene-2-carboxylic acid (188, 30 mg, 24.37 μmol, formic acid salt) in dry acetonitrile (1 mL) and dry THF (1 mL) were added DIPEA (15.75 mg, 121.86 μmol, 21.23 μL) and triphosgene (10.85 mg, 36.56 μmol) at rt. The resulting mixture was stirred at rt for 16 h. The reaction mixture was concentrated and purified by reverse-phase preparative HPLC [Column: X Bridge C 8 (150×19.1 mm) 5 μm; Mobile phase A: 10 MM ammonium acetate in water and Mobile Phase B: MeCN] to afford 3-(carboxymethoxy)-4-chloro-5-[3-[[(4S)-1-[[3-[3-[[1-[1-(2,6-dioxo-3-piperidyl)-2-oxo-benzo[cd]indol-6-yl]-4-piperidyl]-2-oxo-imidazolidin-1-yl]phenyl]methylsulfonyl]-2,2-dimethyl-4-piperidyl]amino]phenyl]thiophene-2-carboxylic acid (189, 11 mg, 10.25 μmol, 42% yield) as yellow solid.

¹H-NMR (400 MHz, DMSO-d6): δ 11.11 (s, 1H), 8.28 (d, J=8.00 Hz, 1H), 8.09 (d, J=6.80 Hz, 1H), 7.85 (t, J=0.80 Hz, 1H), 7.71 (s, 1H), 7.56 (d, J=8.80 Hz, 1H), 7.35 (t, J=8.00 Hz, 1H), 7.17 (t, J=7.60 Hz, 1H), 7.12-6.99 (m, 3H), 6.87 (s, 1H), 6.76 (d, J=7.60 Hz, 1H), 6.65 (d, J=8.80 Hz, 1H), 5.64 (d, J=8.00 Hz, 1H), 5.48-5.39 (m, 1H), 4.60 (s, 2H), 4.41-3.87 (m, 2H), 3.61-3.57 (m, 4H), 3.61-3.57 (m, 4H), 3.00-2.60 (m, 6H), 2.11-2.08 (m, 3H), 1.92-1.81 (m, 3H), 1.46-1.39 (m, 7H), 1.20-1.08 (m, 1H). [2 protons are merged into the solvent peak]

LCMS (ES+): m/z 1038.2 [M+H]⁺.

### Example 190

### 3-(carboxymethoxy)-4-chloro-5-(3-(((75)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylic acid (190)

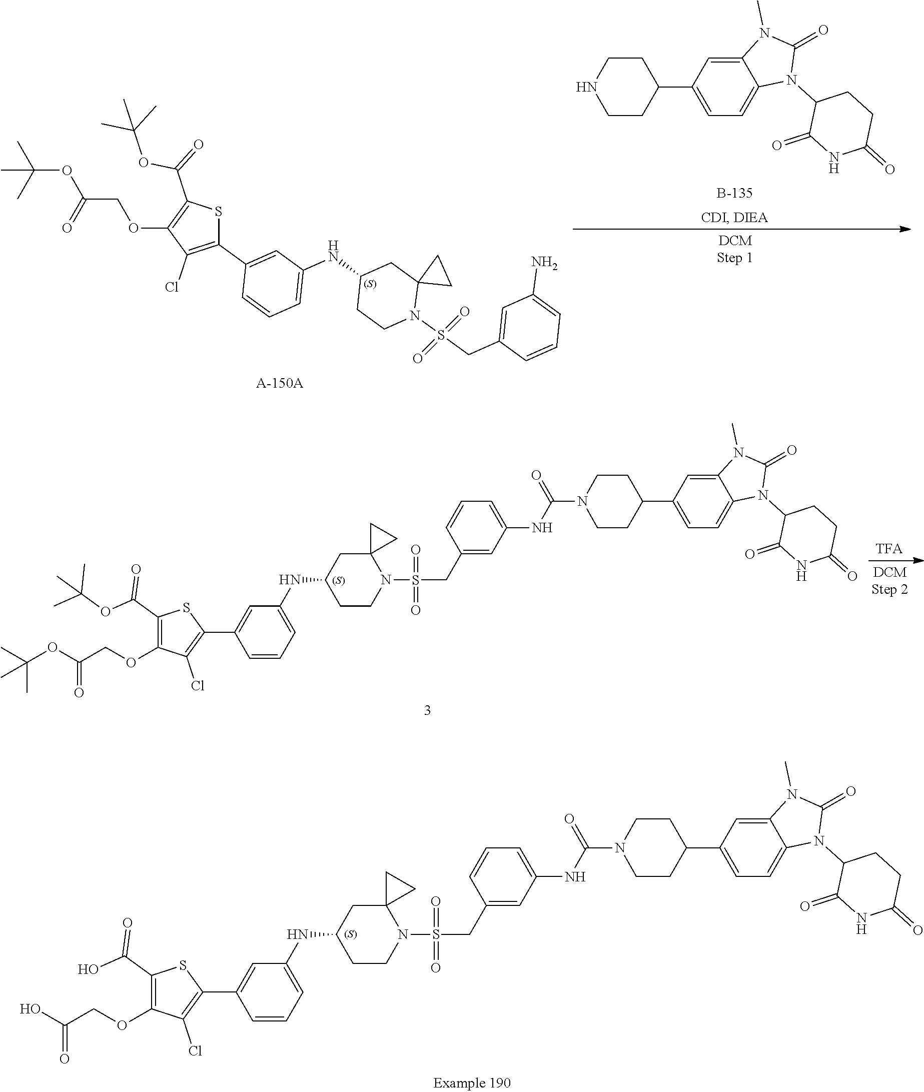

### Step 1: tert-butyl 3-(2-(tert-butoxy)-2-oxoethoxy)-4-chloro-5-(3-(((75)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylate (3)

tert-butyl 3-(2-(tert-butoxy)-2-oxoethoxy)-4-chloro-5-(3-(((7S)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylate (3, 100 mg, 92.02 μmol, 66% yield) was synthesized from(S)-tert-butyl 5-(3-((4-((3-aminobenzyl)sulfonyl)-4-azaspiro[2.5]octan-7-yl)amino)phenyl)-3-(2-(tert-butoxy)-2-oxoethoxy)-4-chlorothiophene-2-carboxylate (A-150A, 100 mg, 139.21 μmol) and 3-(3-methyl-2-oxo-5-(piperidin-4-yl)-2,3-dihydro-1H-benzo[d]imidazol-1-yl)piperidine-2,6-dione (B-135, 47.67 mg, 139.21 μmol) in a similar fashion to Compound B-242. The residue was purified by column chromatography (SiO₂, Petroleum ether:Ethyl acetate=1:0 to 0:1). LCMS (ESI): m/z 1086.8 [M+H]⁺

### Step 2: 3-(carboxymethoxy)-4-chloro-5-(3-(((7S)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylic acid (190)

3-(carboxymethoxy)-4-chloro-5-(3-(((75)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylic acid (190, 25.1 mg, 22.83 μmol, 25% yield, TFA salt) was synthesized from tert-butyl 3-(2-(tert-butoxy)-2-oxoethoxy)-4-chloro-5-(3-(((75)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylate (3, 100 mg, 92.02 μmol) in a similar fashion to Compound A-26. The residue was purified by prep-HPLC (flow: 25 mL/min; gradient: from 72%-42% water (0.1% TFA)-ACN; column: 3_Phenomenex Luna C18 150×25 mm×10 μm.

LCMS (ESI): m/z 974.2 [M+H]⁺

¹H NMR (400 MHz, DMSO-d₆) δ 11.13-11.03 (m, 1H), 8.63-8.55 (m, 1H), 7.49-7.41 (m, 2H), 7.24-7.12 (m, 3H), 7.04-7.00 (m, 1H), 6.96-6.92 (m, 1H), 6.88-6.83 (m, 2H), 6.81-6.77 (m, 1H), 6.71-6.65 (m, 1H), 5.39-5.30 (m, 1H), 4.96-4.86 (m, 2H), 4.34-4.26 (m, 3H), 4.17 (br d, J=14.0 Hz, 1H), 3.60 (br s, 3H), 3.33 (s, 3H), 3.03-2.84 (m, 4H), 2.67 (s, 2H), 2.01-1.94 (m, 2H), 1.83-1.73 (m, 3H), 1.66-1.49 (m, 3H), 1.35-1.29 (m, 1H), 0.66-0.47 (m, 4H).

### Example 191

### 3-(carboxymethoxy)-4-chloro-5-(3-(((4S)-1-((3-(2-(3-((1-(2,6-dioxopiperidin-3-yl)-3-methyl-2-oxo-2,3-dihydro-1H-benzo[d]imidazol-5-yl)amino)phenyl)acetamido)benzyl)sulfonyl)-2,2-dimethylpiperidin-4-yl)amino)phenyl)thiophene-2-carboxylic acid (191)

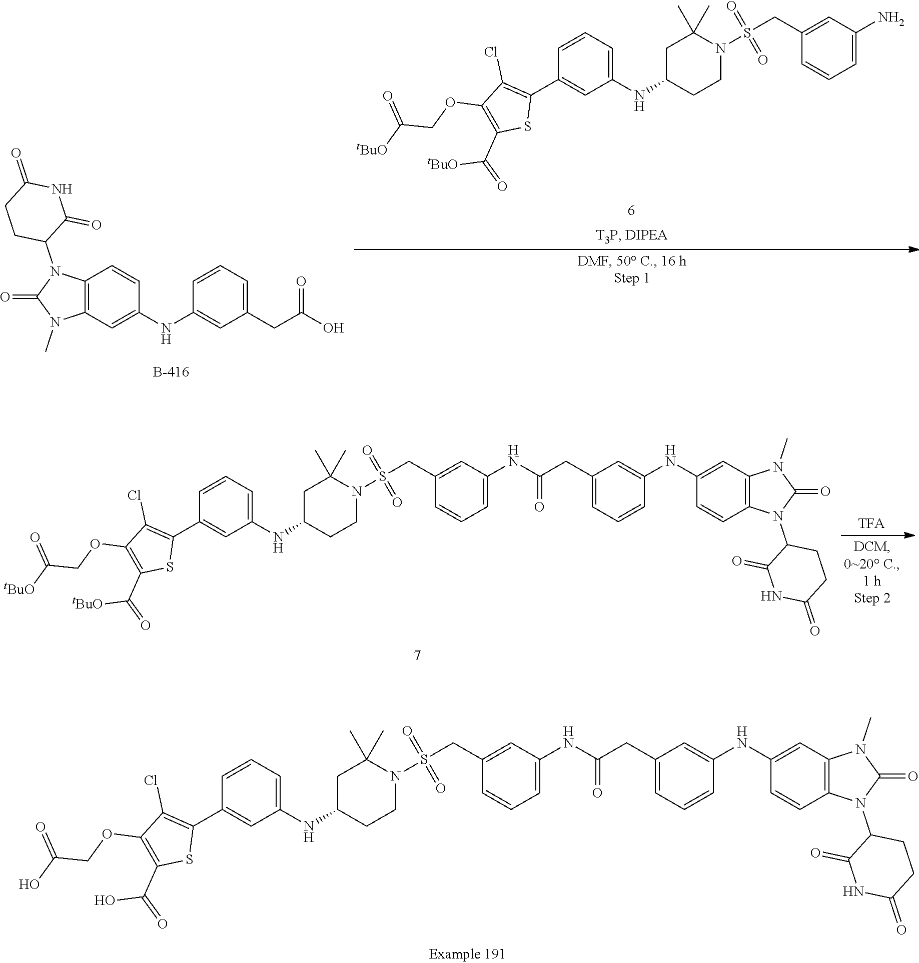 [SMILES: Cn1c(=O)n(C2CCC(=O)NC2=O)c2ccc(Nc3cccc(CC(=O)O)c3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(Nc3cccc(CC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)c3)cc21 ; Cn1c(=O)n(C2CCC(=O)NC2=O)c2ccc(Nc3cccc(CC(=O)Nc4cccc(CS(=O)(=O)N5CC[C@H](Nc6cccc(-c7sc(C(=O)O)c(OCC(=O)O)c7Cl)c6)CC5(C)C)c4)c3)cc21]

### Step 1: tert-butyl 3-(2-(tert-butoxy)-2-oxoethoxy)-4-chloro-5-(3-(((4S)-1-((3-(2-(3-((1-(2,6-dioxopiperidin-3-yl)-3-methyl-2-oxo-2,3-dihydro-1H-benzo[d]imidazol-5-yl)amino)phenyl)acetamido)benzyl)sulfonyl)-2,2-dimethylpiperidin-4-yl)amino)phenyl)thiophene-2-carboxylate (7)

tert-butyl 3-(2-(tert-butoxy)-2-oxoethoxy)-4-chloro-5-(3-(((4S)-1-((3-(2-(3-((1-(2,6-dioxopiperidin-3-yl)-3-methyl-2-oxo-2,3-dihydro-1H-benzo[d]imidazol-5-yl)amino)phenyl)acetamido)benzyl)sulfonyl)-2,2-dimethylpiperidin-4-yl)amino)phenyl)thiophene-2-carboxylate (7, 100 mg, 85.06 μmol, 58% yield) was synthesized from 2-(3-((1-(2,6-dioxopiperidin-3-yl)-3-methyl-2-oxo-2,3-dihydro-1H-benzo[d]imidazol-5-yl)amino)phenyl)acetic acid (B-416, 60 mg, 147 μmol) and(S)-tert-butyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (6, 105.83 mg, 147 μmol) in a similar fashion to Compound A-88. The residue was purified by reverse phase prep HPLC (TFA condition).

¹H NMR (400 MHz, DMSO-d6) δ=11.08 (br s, 1H), 10.21 (s, 1H), 8.01 (s, 1H), 7.69-7.59 (m, 2H), 7.35-7.17 (m, 2H), 7.14-7.03 (m, 3H), 6.99-6.84 (m, 4H), 6.83-6.64 (m, 4H), 5.72-5.65 (m, 1H), 5.36-5.27 (m, 1H), 4.85 (s, 2H), 4.43-4.25 (m, 2H), 3.55 (s, 2H), 3.47-3.42 (m, 1H), 3.25 (s, 3H), 3.12 (br s, 1H), 2.90 (s, 1H), 2.74 (s, 1H), 2.68 (br dd, J=1.8, 3.7 Hz, 2H), 2.06-1.97 (m, 1H), 1.78 (br d, J=11.5 Hz, 2H), 1.52 (s, 9H), 1.47-1.45 (m, 3H), 1.42 (s, 9H), 1.39 (s, 3H), 1.19-1.06 (m, 1H). LCMS (ESI): m/z 1110.6 [M+H]⁺

### Step 2: 3-(carboxymethoxy)-4-chloro-5-(3-(((4S)-1-((3-(2-(3-((1-(2,6-dioxopiperidin-3-yl)-3-methyl-2-oxo-2,3-dihydro-1H-benzo[d]imidazol-5-yl)amino)phenyl)acetamido)benzyl)sulfonyl)-2,2-dimethylpiperidin-4-yl)amino)phenyl)thiophene-2-carboxylic acid (191)

3-(carboxymethoxy)-4-chloro-5-(3-(((4S)-1-((3-(2-(3-((1-(2,6-dioxopiperidin-3-yl)-3-methyl-2-oxo-2,3-dihydro-1H-benzo[d]imidazol-5-yl)amino)phenyl)acetamido)benzyl)sulfonyl)-2,2-dimethylpiperidin-4-yl)amino)phenyl)thiophene-2-carboxylic acid (191, 83.96 mg, 73.66 μmol, 85% yield, TFA salt) was synthesized from tert-butyl 3-(2-(tert-butoxy)-2-oxoethoxy)-4-chloro-5-(3-(((4S)-1-((3-(2-(3-((1-(2,6-dioxopiperidin-3-yl)-3-methyl-2-oxo-2,3-dihydro-1H-benzo[d]imidazol-5-yl)amino)phenyl)acetamido)benzyl)sulfonyl)-2,2-dimethylpiperidin-4-yl)amino)phenyl)thiophene-2-carboxylate (7, 100 mg, 86.45 μmol) in a similar fashion to Compound A-26. The residue was lyophilized (MeCN/H₂O=1:10, 10 mL).

LCMS (ESI): m/z 998.2 [M+H]⁺

¹H NMR (400 MHz, DMSO+D₂O) δ=7.66 (s, 1H), 7.51 (br d, J=8.3 Hz, 1H), 7.30 (t, J=7.8 Hz, 1H), 7.20 (t, J=7.9 Hz, 1H), 7.13-7.04 (m, 2H), 7.02 (s, 1H), 6.93 (s, 1H), 6.89-6.81 (m, 4H), 6.79-6.69 (m, 2H), 6.67 (br d, J=8.3 Hz, 1H), 5.30-5.17 (m, 1H), 4.88 (s, 2H), 4.42-4.16 (m, 2H), 3.52 (s, 2H), 3.50-3.36 (m, 2H), 3.21 (s, 3H), 3.13-3.03 (m, 1H), 2.92-2.79 (m, 1H), 2.70-2.61 (m, 2H), 2.04-1.99 (m, 1H), 1.83 (br d, J=11.9 Hz, 1H), 1.73 (br d, J=11.7 Hz, 1H), 1.41 (s, 4H), 1.34 (s, 3H), 1.15-1.05 (m, 1H).

### Example 122

### 3-(carboxymethoxy)-4-chloro-5-(3-(((4S)-1-((3-(2-(3-(1-(2,6-dioxopiperidin-3-yl)-3-methyl-2-oxo-2,3-dihydro-1H-benzo[d]imidazol-S-yl)phenyl)acetamido)benzyl)sulfonyl)-2,2-dimethylpiperidin-4-yl)amino)phenyl)thiophene-2-carboxylic acid (192)

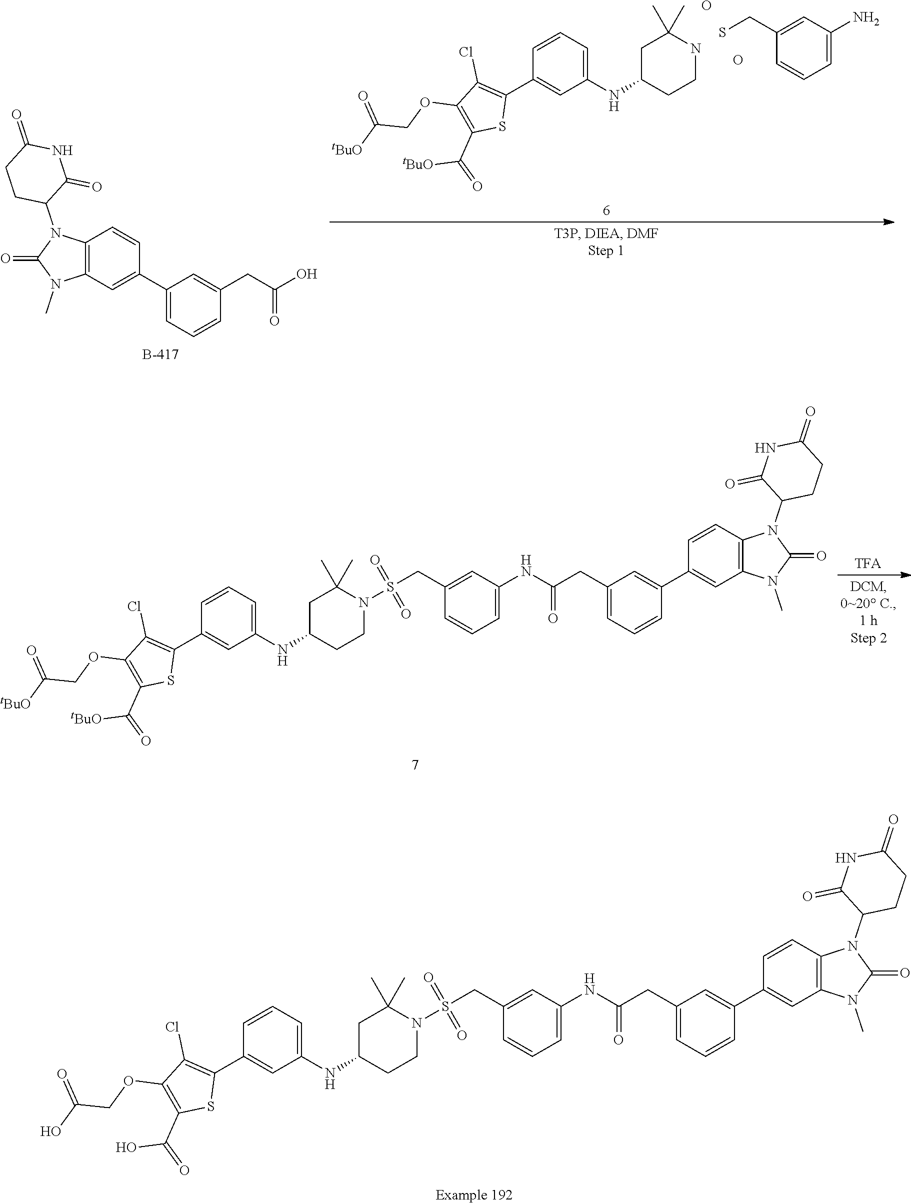

### Step 1: tert-butyl 3-(2-(tert-butoxy)-2-oxoethoxy)-4-chloro-5-(3-(((4S)-1-((3-(2-(3-(1-(2,6-dioxopiperidin-3-yl)-3-methyl-2-oxo-2,3-dihydro-1H-benzo[d]imidazol-5-yl)phenyl)acetamido)benzyl)sulfonyl)-2,2-dimethylpiperidin-4-yl)amino)phenyl)thiophene-2-carboxylate (7)

tert-butyl 3-(2-(tert-butoxy)-2-oxoethoxy)-4-chloro-5-(3-(((4S)-1-((3-(2-(3-(1-(2,6-dioxopiperidin-3-yl)-3-methyl-2-oxo-2,3-dihydro-1H-benzo[d]imidazol-5-yl)phenyl)acetamido)benzyl)sulfonyl)-2,2-dimethylpiperidin-4-yl)amino)phenyl)thiophene-2-carboxylate (7, 80 mg, 68.65 μmol, 68% yield) was synthesized from 2-(3-(1-(2,6-dioxopiperidin-3-yl)-3-methyl-2-oxo-2,3-dihydro-1H-benzo[d]imidazol-5-yl)phenyl)acetic acid (B-417, 40 mg, 101.68 μmol) and(S)-tert-butyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (6, 73.24 mg, 101.68 μmol) in a similar fashion to Compound A-88. The residue was purified by reversed phase column (TFA).

LCMS (EST): m/z 1095.4 [M+H]⁺

### Step 2: 3-(carboxymethoxy)-4-chloro-5-(3-(((4S)-1-((3-(2-(3-(1-(2,6-dioxopiperidin-3-yl)-3-methyl-2-oxo-2,3-dihydro-1H-benzo[d]imidazol-5-yl)phenyl)acetamido)benzyl)sulfonyl)-2,2 dimethylpiperidin-4-yl)amino)phenyl)thiophene-2-carboxylic acid (192)

3-(carboxymethoxy)-4-chloro-5-(3-(((4S)-1-((3-(2-(3-(1-(2,6-dioxopiperidin-3-yl)-3-methyl-2-oxo-2,3-dihydro-1H-benzo[d]imidazol-5-yl)phenyl)acetamido)benzyl)sulfonyl)-2,2-dimethylpiperidin-4-yl)amino)phenyl)thiophene-2-carboxylic acid (192, 45.71 mg, 41.65 μmol, 57% yield, TFA salt) was synthesized from tert-butyl 3-(2-(tert-butoxy)-2-oxoethoxy)-4-chloro-5-(3-(((4S)-1-((3-(2-(3-(1-(2,6-dioxopiperidin-3-yl)-3-methyl-2-oxo-2,3-dihydro-1H-benzo[d]imidazol-5-yl)phenyl)acetamido)benzyl)sulfonyl)-2,2-dimethylpiperidin-4-yl)amino)phenyl)thiophene-2-carboxylate (7, 80 mg, 73.01 μmol) in a similar fashion to Compound A-26. The residue was purified by prep-HPLC (flow: 25 mL/min; gradient: from 72-42% water (0.1% TFA)-ACN; column: Phenomenex Luna C18 75×30 mm×3 μm).

LCMS (ESI): m/z 984.5 [M+H]⁺

¹H NMR (400 MHz, DMSO-d₆) δ 11.12 (s, 1H), 10.28 (s, 1H), 7.67-7.59 (m, 3H), 7.55-7.49 (m, 1H), 7.44-7.34 (m, 3H), 7.33-7.24 (m, 3H), 7.23-7.14 (m, 1H), 7.11-7.05 (m, 1H), 6.90-6.85 (m, 1H), 6.84-6.78 (m, 1H), 6.69-6.63 (m, 1H), 5.40 (dd, J=5.2, 12.4 Hz, 1H), 4.91 (s, 2H), 4.340 (d, J=13.6 Hz, 1H), 4.27 (s, 1H), 3.73 (s, 2H), 3.51-3.42 (m, 2H), 3.16-3.06 (m, 1H), 2.93-2.77 (m, 2H), 2.69-2.63 (m, 1H), 2.11-2.01 (m, 1H), 1.92-1.72 (m, 2H), 1.48-1.33 (m, 7H), 1.12-1.10 (m, 1H).

### Example 193

### 3-(carboxymethoxy)-4-chloro-5-(3-(((4R)-1-((3-(4-(3-(2,6-dioxopiperidin-3-yl) pyrazolo[1,5-a]pyridin-6-yl)piperidine-1-carboxamido)benzyl)sulfonyl)-2,2-dimethylpiperidin-4-yl)amino)phenyl)thiophene-2-carboxylic acid (193)

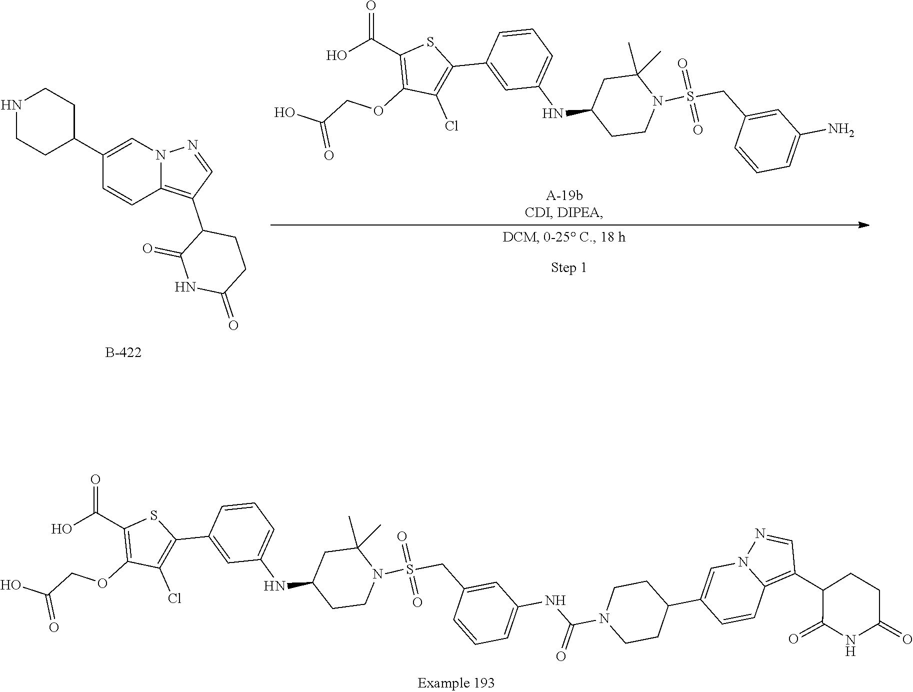 [SMILES: O=C1CCC(c2cnn3cc(C4CCNCC4)ccc23)C(=O)N1 ; CC1(C)C[C@H](Nc2cccc(-c3sc(C(=O)O)c(OCC(=O)O)c3Cl)c2)CCN1S(=O)(=O)Cc1cccc(N)c1 ; CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c(C5CCC(=O)NC5=O)cnn4c3)CC2)c1]

### Step 1: 3-(carboxymethoxy)-4-chloro-5-(3-(((4R)-1-((3-(4-(3-(2,6-dioxopiperidin-3-yl) pyrazolo[1,5-a]pyridin-6-yl)piperidine-1-carboxamido)benzyl)sulfonyl)-2,2-dimethylpiperidin-4-yl)amino)phenyl)thiophene-2-carboxylic acid (193)

3-(carboxymethoxy)-4-chloro-5-(3-(((4R)-1-((3-(4-(3-(2,6-dioxopiperidin-3-yl) pyrazolo[1,5-a]pyridin-6-yl)piperidine-1-carboxamido)benzyl)sulfonyl)-2,2-dimethylpiperidin-4-yl)amino)phenyl)thiophene-2-carboxylic acid (193, 18.02 mg, 16.82 μmol, 20% yield, TFA salt) was synthesized from (R)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19b, 52.30 mg, 86.00 μmol) and 3-(6-(piperidin-4-yl) pyrazolo[1,5-a]pyridin-3-yl)piperidine-2,6-dione (B-422, 30 mg, 86.00 μmol, HCl salt) in a similar fashion to Compound B-242. The mixture was purified by prep-HPLC (flow: 27 mL/min; gradient: from 36-66% MeCN in water (0.1% TFA) over 10 min; column: Phenomenex Synergi C18 150×25 mm×10 μm).

LCMS (ESI): m/z 946.3 [M+H]⁺

¹H NMR (400 MHz, d₆-DMSO) δ 10.82 (s, 1H), 8.63 (s, 1H), 8.47 (s, 1H), 7.84 (s, 1H), 7.58-7.55 (m, 2H), 7.48-7.45 (m, 1H), 7.26-7.16 (m, 3H), 6.99-6.96 (m, 1H), 6.86 (s, 1H), 6.81-6.77 (m, 1H), 6.69-6.67 (m, 1H), 4.91 (s, 2H), 4.38-4.26 (m, 4H), 4.16-4.12 (m, 1H), 3.53-3.45 (m, 2H), 3.23-3.12 (m, 2H), 3.12-2.90 (m, 3H), 2.80-2.66 (m, 1H), 2.59-2.56 (m, 1H), 2.33-2.32 (m, 1H), 2.12-2.06 (m, 1H), 1.89-1.79 (m, 3H), 1.69-1.62 (m, 2H), 1.48 (s, 3H), 1.39 (s, 3H), 1.24-1.07 (m, 2H).

### Example 194

### 3-(carboxymethoxy)-4-chloro-5-[3-[[(4S)-1-[3-[2-[4-[1-(2,6-dioxo-3-piperidyl)-3-methyl-2-oxo-benzimidazol-5-yl]pyrazol-1-yl]acetyl]amino]phenyl]methylsulfonyl]-2,2-dimethyl-4-piperidyl]amino]phenyl]thiophene-2-carboxylic acid (194)

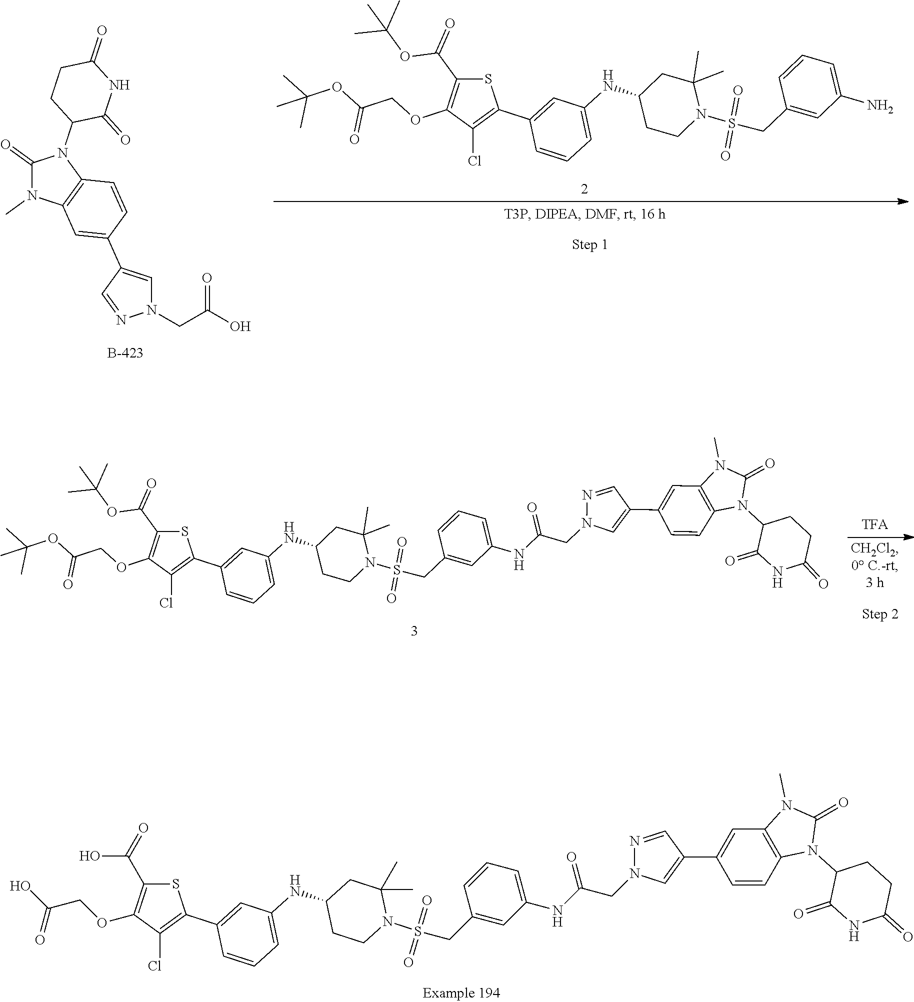 [SMILES: Cn1c(=O)n(C2CCC(=O)NC2=O)c2ccc(-c3cnn(CC(=O)O)c3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(-c3cnn(CC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)c3)cc21 ; Cn1c(=O)n(C2CCC(=O)NC2=O)c2ccc(-c3cnn(CC(=O)Nc4cccc(CS(=O)(=O)N5CC[C@H](Nc6cccc(-c7sc(C(=O)O)c(OCC(=O)O)c7Cl)c6)CC5(C)C)c4)c3)cc21]

### Step 1: tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (3)

tert-butyl 3-(2-tert-butoxy-2-oxo-ethoxy)-4-chloro-5-[3-[[(4S)-1-[[3-[[2-[4-[1-(2,6-dioxo-3-piperidyl)-3-methyl-2-oxo-benzimidazol-5-yl]pyrazol-1-yl]acetyl]amino]phenyl]methylsulfonyl]-2,2-dimethyl-4-piperidyl]amino]phenyl]thiophene-2-carboxylate (400 mg, 121.58 μmol, 28% yield, 33% purity) was synthesized from 2-[4-[1-(2,6-dioxo-3-piperidyl)-3-methyl-2-oxo-benzimidazol-5-yl]pyrazol-1-yl]acetic acid (B-423, 300 mg, 434.28 μmol, TFA salt) and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (312.82 mg, 434.28 μmol) in a similar fashion to Compound A-88. The solvent was removed under reduced pressure to afford as a dark oil.

LCMS (ES−): m/z 1084.0 [M−H]⁻

### Step 2: 3-(carboxymethoxy)-4-chloro-5-[3-[[(4S)-1-[[3-[[2-[4-[1-(2,6-dioxo-3-piperidyl)-3-methyl-2-oxo-benzimidazol-5-yl]pyrazol-1-yl]acetyl]amino]phenyl]methylsulfonyl]-2,2-dimethyl-4-piperidyl]amino]phenyl]thiophene-2-carboxylic acid (194)

3-(carboxymethoxy)-4-chloro-5-[3-[[(4S)-1-[[3-[[2-[4-[1-(2,6-dioxo-3-piperidyl)-3-methyl-2-oxo-benzimidazol-5-yl]pyrazol-1-yl]acetyl]amino]phenyl]methylsulfonyl]-2,2-dimethyl-4-piperidyl]amino]phenyl]thiophene-2-carboxylic acid (194, 105 mg, 99.39 μmol, 84% yield, TFA salt) was synthesized from tert-butyl 3-(2-tert-butoxy-2-oxo-ethoxy)-4-chloro-5-[3-[[(4S)-1-[[3-[[2-[4-[1-(2,6-dioxo-3-piperidyl)-3-methyl-2-oxo-benzimidazol-5-yl]pyrazol-1-yl]acetyl]amino]phenyl]methylsulfonyl]-2,2-dimethyl-4-piperidyl]amino]phenyl]thiophene-2-carboxylate (400 mg, 117.90 μmol) in a similar fashion to Compound A-26. The residue was purified by reverse-phase preparative HPLC [Column: X-BRIDGE C8 (150×19) mm, 5 microns; 0.1% Formic acid in water:CH₃CN).

¹H-NMR (400 MHz, DMSO-d6): δ 11.13 (s, 1H), 10.47 (s, 1H), 8.20 (s, 1H), 7.94 (d, J=Hz, 1H), 7.69 (s, 1H), 7.63 (d, J=8.00 Hz, 1H), 7.46 (s, 1H), 7.35 (t, J=7.60 Hz, 1H), 7.28 (d, J=8.40 Hz, 1H), 7.22-7.10 (m, 3H), 6.88 (s, 1H), 6.79 (d, J=7.60 Hz, 1H), 6.68 (d, J=7.20 Hz, 1H), 5.71-5.69 (m, 1H), 5.37 (dd, J=5.60, 12.60 Hz, 1H), 5.06 (s, 2H), 4.90 (s, 2H), 4.45-4.29 (m, 2H), 3.52-3.44 (m, 2H), 2.98 (t, J=12.40 Hz, 2H), 2.91-2.87 (m, 1H), 2.78-2.68 (m, 1H), 2.05-2.02 (m, 1H), 1.89-1.77 (m, 3H), 1.46-1.35 (m, 9H), 1.13-1.08 (m, 1H). LCMS (ES+): m/z 972.2 [M+H]⁺.

### Example 195

### 3-(carboxymethoxy)-4-chloro-5-[3-[[(4S)-1-[3-[4-[9-(2,6-dioxo-3-piperidyl)pyrido[2,3-b]indol-6-yl]piperidine-1-carbonyl]amino]phenyl]methylsulfonyl]-2,2-dimethyl-4-piperidyl]amino]phenyl]thiophene-2-carboxylic acid (195)

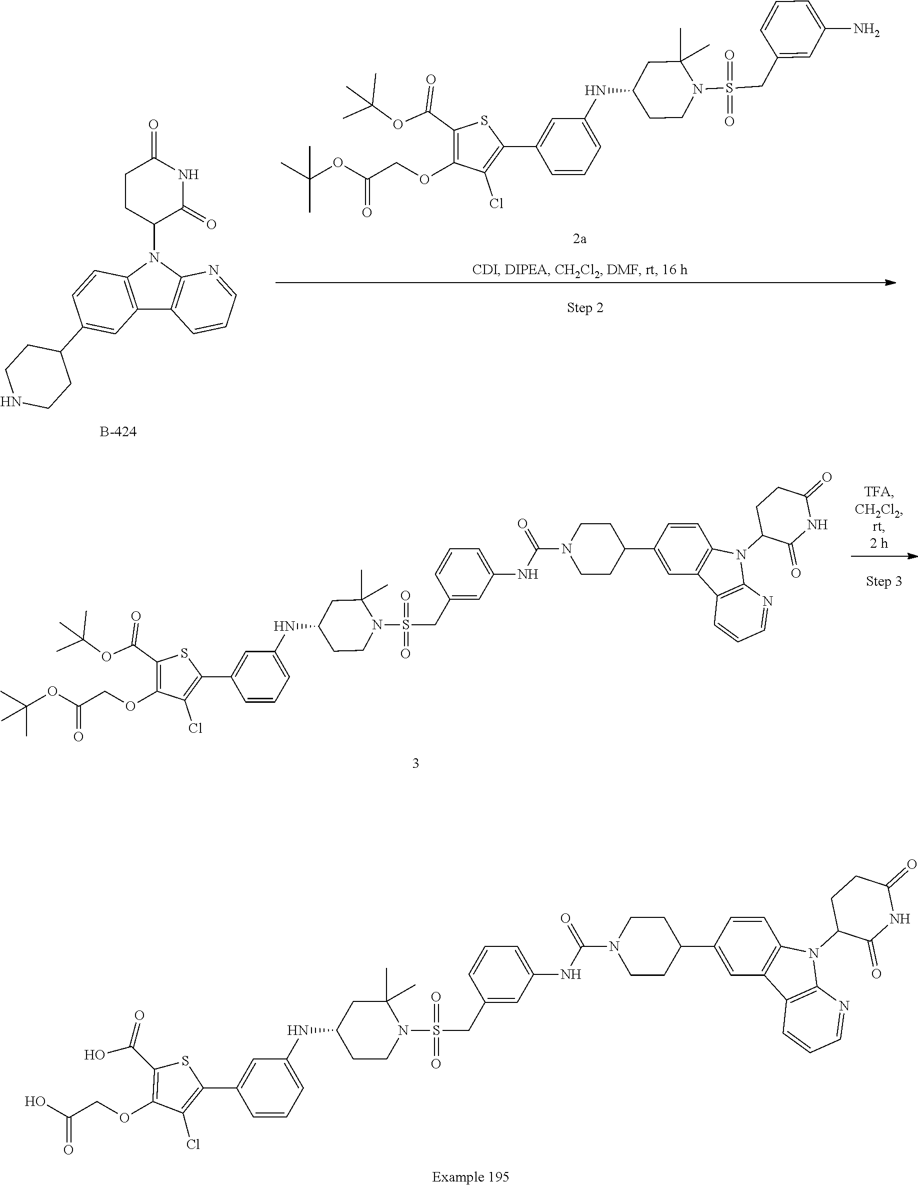 [SMILES: O=C1CCC(n2c3ccc(C4CCNCC4)cc3c3cccnc32)C(=O)N1 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)N5CCC(c6ccc7c(c6)c6cccnc6n7C6CCC(=O)NC6=O)CC5)c4)C(C)(C)C3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)c3cccnc3n4C3CCC(=O)NC3=O)CC2)c1]

### Step 2: tert-butyl 3-(2-tert-butoxy-2-oxo-ethoxy)-4-chloro-5-[3-[[(4S)-1-[[3-[[4-[9-(2,6-dioxo-3-piperidyl)pyrido[2,3-b]indol-6-yl]piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[[3-[[4-[9-(2,6-dioxo-3-piperidyl)pyrido[2,3-b]indol-6-yl]piperidine-1-carbonyl]amino]phenyl]methylsulfonyl]-2,2-dimethyl-4-piperidyl]amino]phenyl]thiophene-2-carboxylate (3, 200 mg, 94.97 μmol, 56% yield, 53% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2a, 152.52 mg, 170.01 μmol, TFA salt) and 3-[6-(4-piperidyl)pyrido[2,3-b]indol-9-yl]piperidine-2,6-dione (B-424, 100 mg, 170.01 μmol, TFA salt) in a similar fashion to Compound B-117. The residue was diluted by water (2.5 mL). The solid was filtered and dried to give the product as a brown solid. LCMS (ES+): m/z 1108.2 [M+H]⁺

### Step 3: 3-(carboxymethoxy)-4-chloro-5-[3-[[(4S)-1-[[3-[[4-[9-(2,6-dioxo-3-piperidyl)pyrido[2,3-b]indol-6-yl]piperidine-1-carbonyl]amino]phenyl]methylsulfonyl]-2,2-dimethyl-4-piperidyl]amino]phenyl]thiophene-2-carboxylic acid (195)

3-(carboxymethoxy)-4-chloro-5-[3-[[(4S)-1-[[3-[[4-[9-(2,6-dioxo-3-piperidyl)pyrido[2,3-b]indol-6-yl]piperidine-1-carbonyl]amino]phenyl]methylsulfonyl]-2,2-dimethyl-4-piperidyl]amino]phenyl]thiophene-2-carboxylic acid (195, 40 mg, 36.09 μmol, 38% yield, formic acid salt) was synthesized from tert-butyl 3-(2-tert-butoxy-2-oxo-ethoxy)-4-chloro-5-[3-[[(4S)-1-[[3-[[4-[9-(2,6-dioxo-3-piperidyl)pyrido[2,3-b]indol-6-yl]piperidine-1-carbonyl]amino]phenyl]methylsulfonyl]-2,2-dimethyl-4-piperidyl]amino]phenyl]thiophene-2-carboxylate (3, 200.00 mg, 94.97 μmol) in a similar fashion to Compound A-26. The residue was purified by reverse phase preparatory HPLC [Purification method: Column: Xselect C18 (250×19) mm, 5 micron; Mobile phase A: 0.1% Formic acid in water; Mobile phase B: Acetonitrile.

LCMS (ES+): m/z 998.7 [M+H]⁺

¹H-NMR (400 MHz, DMSO-d6): δ 11.14 (s, 1H), 8.65 (s, 1H), 8.58-8.55 (m, 1H), 8.42-8.40 (m, 1H), 8.15 (s, 1H), 7.60 (s, 1H), 7.55-7.42 (m, 3H), 7.27-7.18 (m, 3H), 7.00 (d, J=7.60 Hz, 1H), 6.88 (s, 1H), 6.81 (d, J=7.60 Hz, 1H), 6.71-6.68 (m, 1H), 6.03-6.01 (m, 1H), 5.68 (s, 1H), 4.91 (s, 2H), 4.39-4.25 (m, 5H), 3.54-3.46 (m, 3H), 3.18-2.93 (m, 6H), 2.73-2.67 (m, 1H), 2.18-2.07 (m, 1H), 1.92-1.71 (m, 6H), 1.50-1.41 (m, 7H), 1.15-1.05 (m, 1H).

### Example 196

### 3-(carboxymethoxy)-4-chloro-5-(3-(((4S)-1-((3-(4-(1-(2,6-dioxopiperidin-3-yl)-1H-indol-5-yl)piperidine-1-carboxamido)benzyl)sulfonyl)-2,2-dimethylpiperidin-4-yl)amino)phenyl)thiophene-2-carboxylic acid (196)

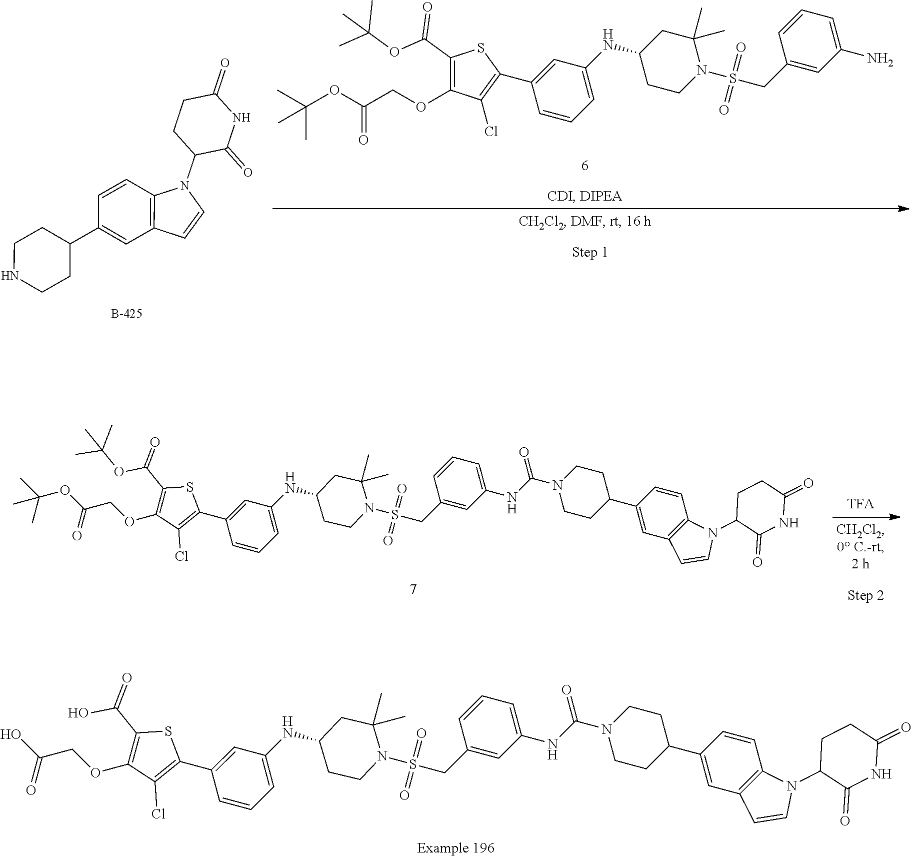

### Step 1: tert-butyl 3-(2-(tert-butoxy)-2-oxoethoxy)-4-chloro-5-(3-(((4S)-1-((3-(4-(1-(2,6-dioxopiperidin-3-yl)-1H-indol-5-yl)piperidine-1-carboxamido)benzyl)sulfonyl)-2,2-dimethylpiperidin-4-yl)amino)phenyl)thiophene-2-carboxylate (7)

tert-butyl 3-(2-tert-butoxy-2-oxo-ethoxy)-4-chloro-5-[3-[[(4S)-1-[[3-[[4-[1-(2,6-dioxo-3-piperidyl) indol-5-yl]piperidine-1-carbonyl]amino]phenyl]methylsulfonyl]-2,2-dimethyl-4-piperidyl]amino]phenyl]thiophene-2-carboxylate (7, 280 mg, 94.51 μmol, 46% yield, 36% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (6, 150 mg, 206.15 μmol) and 3-[S-(4-piperidyl) indol-1-yl]piperidine-2,6-dione (B-425, 76.23 mg, 195.85 μmol) in a similar fashion to Compound B-242. The solvent was evaporated under reduced pressure to afford a brown gummy solid. LCMS (ES+): m/z 1057.2 [M+H]⁺

### Step 2: 3-(carboxymethoxy)-4-chloro-5-(3-(((4S)-1-((3-(4-(1-(2,6-dioxopiperidin-3-yl)-1H-indol-5-yl)piperidine-1-carboxamido)benzyl)sulfonyl)-2,2-dimethylpiperidin-4-yl)amino)phenyl)thiophene-2-carboxylic acid (196)

3-(carboxymethoxy)-4-chloro-5-[3-[[(4S)-1-[3-[4-[1-(2,6-dioxo-3-piperidyl) indol-5-yl]piperidine-1-carbonyl]amino]phenyl]methylsulfonyl]-2,2-dimethyl-4-piperidyl]amino]phenyl]thiophene-2-carboxylic acid (196, 12 mg, 11.18 μmol, 14% yield, formic acid salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl) indol-5-yl]piperidine-1-carbonyl]amino]phenyl]methylsulfonyl]-2,2-dimethyl-4-piperidyl]amino]phenyl]thiophene-2-carboxylate (7, 280 mg, 82.06 μmol) in a similar fashion to Compound A-26. The residue was purified by reverse-phase preparative HPLC [Column: XSELECT-C-18, 19.1×150 mm, 5 μm; Mobile phase A: 0.1% Formic acid in water and Mobile Phase B: MeCN].

LCMS (ES+): m/z 945.0 [M+H]⁺

¹H-NMR (400 MHz, DMSO-d6): δ 11.06 (s, 1H), 8.63 (s, 1H), 7.58 (s, 1H), 7.48 (d, J=8.00 Hz, 1H), 7.38 (s, 1H), 7.34-7.34 (m, 2H), 7.25 (t, J=7.60 Hz, 1H), 7.18 (t, J=7.60 Hz, 1H), 7.05 (d, J=8.40 Hz, 1H), 6.99 (d, J=7.60 Hz, 1H), 6.84 (s, 1H), 6.78 (d, J=7.60 Hz, 1H), 6.66 (d, J=8.00 Hz, 1H), 6.43 (d, J=3.20 Hz, 1H), 5.70-5.55 (m, 2H), 4.66 (s, 1H), 4.38-4.24 (m, 4H), 3.52-3.45 (m, 2H), 3.16-3.13 (m, 3H), 2.95-2.88 (m, 3H), 2.21-2.10 (m, 4H), 1.91-1.71 (m, 4H), 1.70-1.58 (m, 2H), 1.50-1.35 (m, 7H), 1.20-1.01 (m, 1H).

### Example 197 and Example 198

### 3-(carboxymethoxy)-4-chloro-5-[3-[[(4S)-1-[[3-[2-[[1-[1-(2,6-dioxo-3-piperidyl)-3-methyl-2-oxo-benzimidazol-5-yl]-4-piperidyl]amino]ethylamino]phenyl]methylsulfonyl]-2,2-dimethyl-4-piperidyl]amino]phenyl]thiophene-2-carboxylic acid (197) and 3-(carboxymethoxy)-4-chloro-5-[3-[[(4S)-1-[3-[3-[[1-[1-(2,6-dioxo-3-piperidyl)-3-methyl-2-oxo-benzimidazol-5-yl]-4-piperidyl]-2-oxo-imidazolidin-1-yl]phenyl]methylsulfonyl]-2,2-dimethyl-4-piperidyl]amino]phenyl]thiophene-2-carboxylic acid (198)

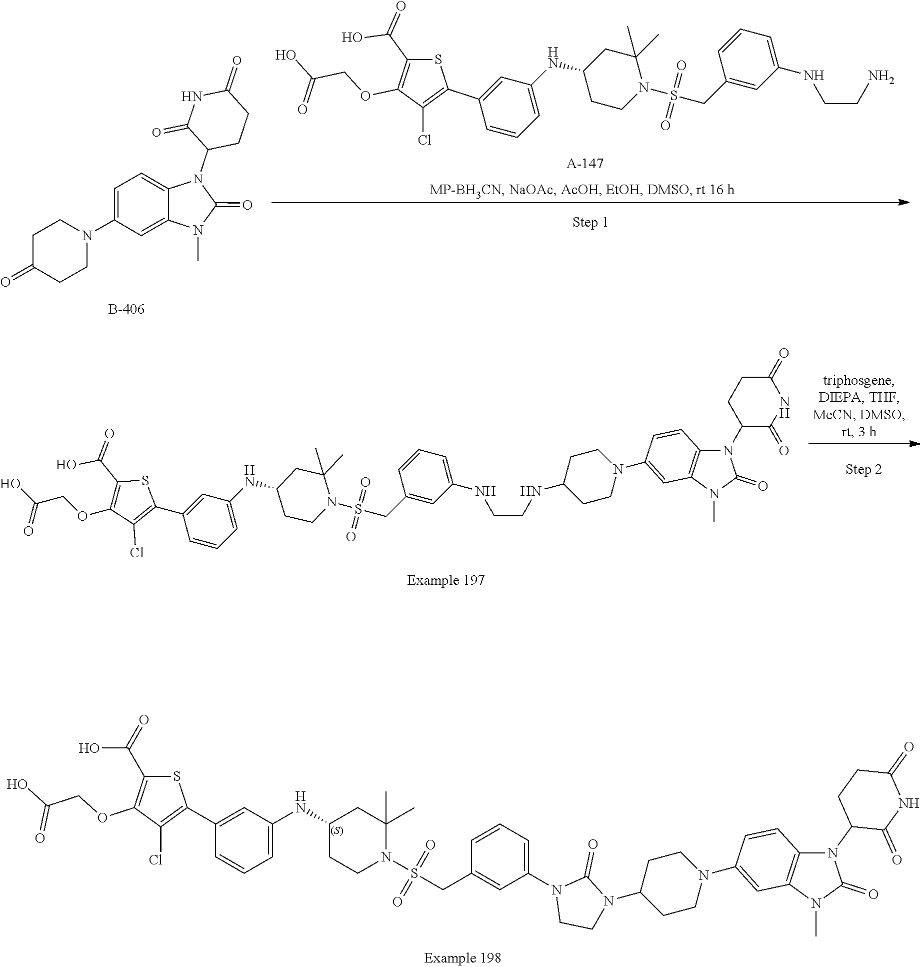

### Step 1: 3-(carboxymethoxy)-4-chloro-5-[3-[[(4S)-1-[[3-[2-[[1-[1-(2,6-dioxo-3-piperidyl)-3-methyl-2-oxo-benzimidazol-5-yl]-4-piperidyl]amino]ethylamino]phenyl]methylsulfonyl]-2,2-dimethyl-4-piperidyl]amino]phenyl]thiophene-2-carboxylic acid (197)

3-(carboxymethoxy)-4-chloro-5-[3-[[(4S)-1-[3-[2-[[1-[1-(2,6-dioxo-3-piperidyl)-3-methyl-2-oxo-benzimidazol-5-yl]-4-piperidyl]amino]ethylamino]phenyl]methylsulfonyl]-2,2-dimethyl-4-piperidyl]amino]phenyl]thiophene-2-carboxylic acid (197, 250 mg, 230.73 μmol, 34% yield, Formic acid salt) was synthesized from 5-[3-[[(4S)-1-[[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147, 0.5 g, 607.67 μmol, TFA salt) and 3-[3-methyl-2-oxo-5-(4-oxo-1-piperidyl)benzimidazol-1-yl]piperidine-2,6-dione (B-405, 811.78 mg, 615.03 μmol, 27% purity) in a similar fashion to Compound A-74a/b, except using EtOH/DMSO as solvent and including 1.0 eq. NaOAc. The residue was subjected to purification by reverse phase column chromatography [Purification method: Silicycle C18 column; Mobile phase A: 0.1% TFA in water; Mobile phase B: MeCN].

LCMS (ES+): m/z 992.0 [M+H]⁺

¹HNMR (400 MHz, DMSO-d6): δ 11.08 (s, 1H), 8.55 (bs, 1H), 7.20-7.13 (m, 2H), 6.96-6.93 (m, 1H), 6.87-6.80 (m, 3H), 6.78-6.63 (m, 5H), 5.82 (s, 1H), 5.72 (d, J=7.60 Hz, 1H), 5.31-5.28 (m, 1H), 4.63 (s, 2H), 4.26 (dd, J=13.60, 38.80 Hz, 2H), 3.70 (d, J=11.20 Hz, 2H), 3.50-3.34 (m, 3H), 3.30-3.11 (m, 4H), 2.98-2.81 (m, 1H), 2.72-2.60 (m, 4H), 2.07-0.90 (m, 4H), 1.81-1.78 (m, 1H), 1.68-1.62 (m, 2H), 1.48 (s, 3H), 1.41-1.38 (m, 4H), 1.15-1.05 (m, 1H).

### Step 2: 3-(carboxymethoxy)-4-chloro-5-[3-[[(4S)-1-[[3-[3-[[1-[1-(2,6-dioxo-3-piperidyl)-3-methyl-2-oxo-benzimidazol-5-yl]-4-piperidyl]-2-oxo-imidazolidin-1-yl]phenyl]methylsulfonyl]-2,2-dimethyl-4-piperidyl]amino]phenyl]thiophene-2-carboxylic acid (198)

3-(carboxymethoxy)-4-chloro-5-[3-[[(4S)-1-[[3-[3-[[1-[1-(2,6-dioxo-3-piperidyl)-3-methyl-2-oxo-benzimidazol-5-yl]-4-piperidyl]-2-oxo-imidazolidin-1-yl]phenyl]methylsulfonyl]-2,2-dimethyl-4-piperidyl]amino]phenyl]thiophene-2-carboxylic acid (198, 10 mg, 8.70 μmol, 8% yield, Formic acid salt) was synthesized from 3-(carboxymethoxy)-4-chloro-5-[3-[[(4S)-1-[3-[2-[[1-[1-(2,6-dioxo-3-piperidyl)-3-methyl-2-oxo-benzimidazol-5-yl]-4-piperidyl]amino]ethylamino]phenyl]methylsulfonyl]-2,2-dimethyl-4-piperidyl]amino]phenyl]thiophene-2-carboxylic acid (197, 150 mg, 115.65 μmol, Formic acid salt) in a similar fashion to Example 142, except using THE/Acetonitrile/DMSO as the solvent. The residue was then purified by reverse phase prep HPLC [Purification method: Column: X-Bridge C18 (150×19) mm, 5 micron; Mobile phase A: 0.1% Formic acid in water; Mobile phase B: MeCN. LCMS (ES−): m/z 1015.0 [M−H]⁻

¹HNMR (400 MHz, DMSO-d6): δ 11.08 (s, 1H), 7.67 (s, 1H), 7.55 (d, J=7.20 Hz, 1H), 7.33 (t, J=7.60 Hz, 1H), 7.20 (t, J=7.60 Hz, 1H), 7.05 (d, J=7.20 Hz, 1H), 6.95 (d, J=8.40 Hz, 1H), 6.87-6.67 (m, 5H), 5.70 (s, 1H), 5.30-5.27 (m, 1H), 4.86 (s, 2H), 4.35 (dd, J=13.60, 48.20 Hz, 2H), 3.81-3.67 (m, 6H), 2.90-2.64 (m, 8H), 2.09-1.75 (m, 6H), 1.45 (s, 3H), 1.38 (s, 3H), 1.10-1.01 (m, 1H).

### Example 199

### 3-(carboxymethoxy)-4-chloro-5-[3-[[(4S)-1-[[3-[[4-[3-(2,6-dioxo-3-piperidyl)-1-isopropyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylic acid (199)

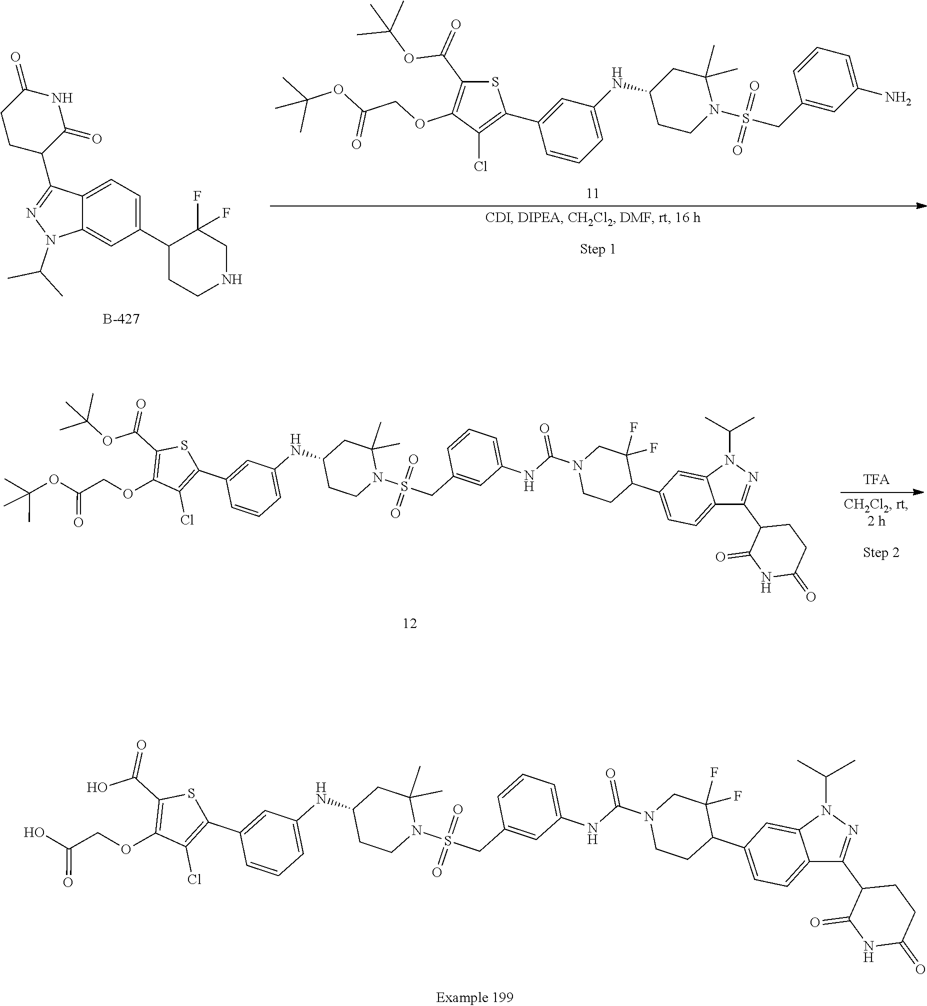 [SMILES: CC(C)n1nc(C2CCC(=O)NC2=O)c2ccc(C3CCNCC3(F)F)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)n1nc(C2CCC(=O)NC2=O)c2ccc(C3CCN(C(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3(F)F)cc21 ; CC(C)n1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21]

### Step 1: tert-butyl 3-(2-tert-butoxy-2-oxo-ethoxy)-4-chloro-5-[3-[[(4S)-1-[[3-[[4-[3-(2,6-dioxo-3-piperidyl)-1-isopropyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylate (12)

tert-butyl 3-(2-tert-butoxy-2-oxo-ethoxy)-4-chloro-5-[3-[[(4S)-1-[[3-[[4-[3-(2,6-dioxo-3-piperidyl)-1-isopropyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylate (12, 135 mg, 96.31 μmol, 51% yield) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxy late (11, 137.03 mg, 188.32 μmol) and 3-[6-(3,3-difluoro-4-piperidyl)-1-isopropyl-indazol-3-yl]piperidine-2,6-dione (B-427, 100 mg, 188.32 μmol, TFA salt) in a similar fashion to Compound B-242. The residue was triturated with MTBE (15 mL) to afford as an off-white solid. LCMS (ES+): m/z 1136.2 [M+H]⁺

### Step 2: 3-(carboxymethoxy)-4-chloro-5-[3-[[(4S)-1-[[3-[[4-[3-(2,6-dioxo-3-piperidyl)-1-isopropyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylic acid (199)

3-(carboxymethoxy)-4-chloro-5-[3-[[(4S)-1-[3-[4-[3-(2,6-dioxo-3-piperidyl)-1-isopropyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylic acid (199, 37 mg, 32.07 μmol, 35% yield, TFA salt) was synthesized from tert-butyl 3-(2-tert-butoxy-2-oxo-ethoxy)-4-chloro-5-[3-[[(4S)-1-[[3-[[4-[3-(2,6-dioxo-3-piperidyl)-1-isopropyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylate (12, 130 mg, 92.75 μmol) in a similar fashion to Compound A-26. The residue was purified by reverse-phase preparative HPLC [Column: X-select C18 (150×19) mm, 5 μm; Mobile Phase A: 0.1% TFA water and Mobile Phase B: MeCN.

¹H-NMR (400 MHz, DMSO-d6): δ 10.89 (s, 1H), 8.82 (s, 1H), 7.65-7.59 (m, 3H), 7.51 (d, J=8.00 Hz, 1H), 7.28 (t, J=8.00 Hz, 1H), 7.20 (t, J=8.40 Hz, 1H), 7.08 (d, J=8.40 Hz, 1H), 7.03 (d, J=7.20 Hz, 1H), 6.88 (s, 1H), 6.81 (d, J=7.60 Hz, 1H), 6.70 (d, J=8.00 Hz, 1H), 4.99-4.94 (m, 1H), 4.92 (s, 2H), 4.68-4.55 (m, 1H), 4.41-4.26 (m, 4H), 3.18-3.09 (m, 2H), 2.71-2.56 (m, 2H), 2.51-2.13 (m, 4H), 1.96-1.79 (m, 3H), 1.51-1.41 (m, 13H), 1.20-1.08 (m, 1H). [3 protons are merged into the solvent peak] LCMS (ES+): m/z 1025.2 [M+H]⁺.

### Example 200

### 3-(carboxymethoxy)-4-chloro-5-[3-[[(4S)-1-[[3-[[4-[3-(2,6-dioxo-3-piperidyl)-1-ethyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylic acid (200)

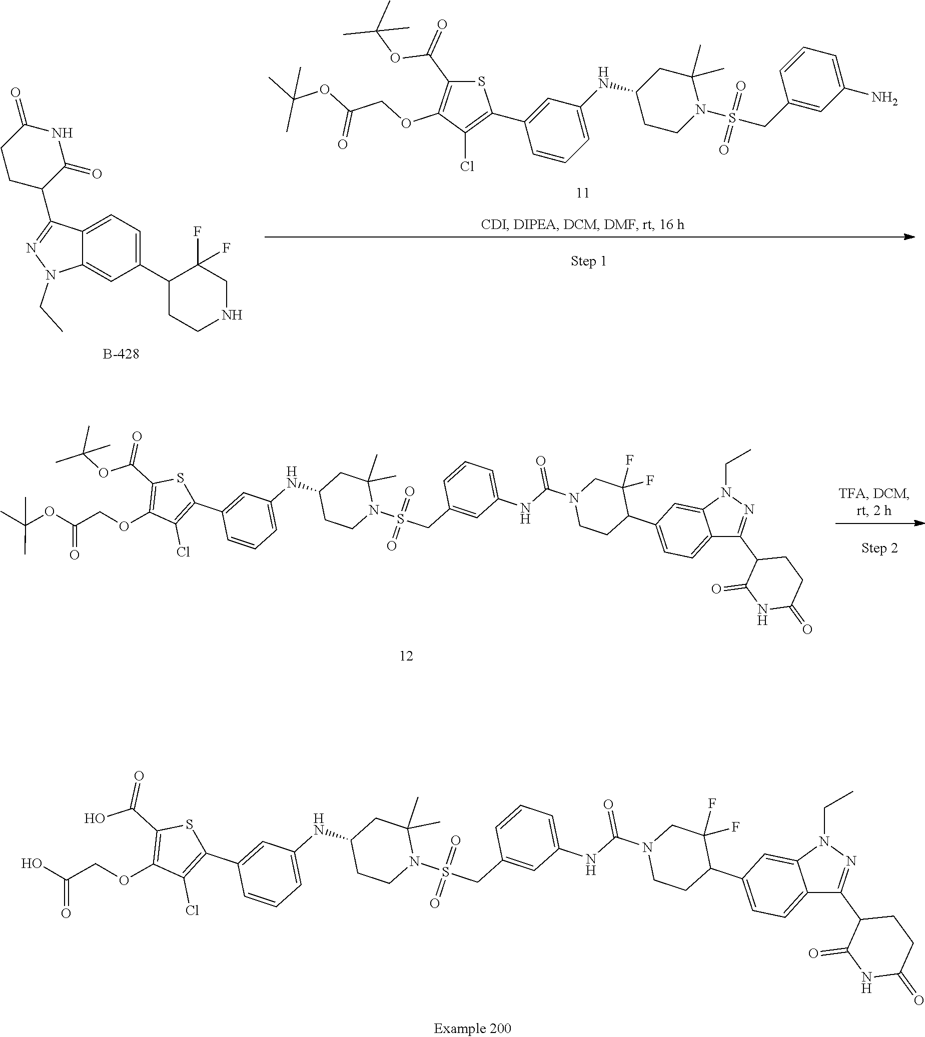 [SMILES: CCn1nc(C2CCC(=O)NC2=O)c2ccc(C3CCNCC3(F)F)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CCn1nc(C2CCC(=O)NC2=O)c2ccc(C3CCN(C(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3(F)F)cc21 ; CCn1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21]

### Step 1: tert-butyl 3-(2-tert-butoxy-2-oxo-ethoxy)-4-chloro-5-[3-[[(4S)-1-[[3-[[4-[3-(2,6-dioxo-3-piperidyl)-1-ethyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylate (12)

tert-butyl 3-(2-tert-butoxy-2-oxo-ethoxy)-4-chloro-5-[3-[[(4S)-1-[[3-[[4-[3-(2,6-dioxo-3-piperidyl)-1-ethyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylate (12, 230 mg, 109.47 μmol, 53% yield, 55% purity, HCl salt) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (11, 150 mg, 206.15 μmol) and 3-[6-(3,3-difluoro-4-piperidyl)-1-ethyl-indazol-3-yl]piperidine-2,6-dione (B-428, 117.34 mg, 247.38 μmol, Formic acid salt) in a similar fashion to Compound B-242. The residue was treated with 1.5N HCl (30 mL). The solid was filtered and dried to afford the product as an off-white solid. The material was used in the next step without further purification. LCMS (ES+): m/z 1122.2 [M+H]⁺

### Step 2: 3-(carboxymethoxy)-4-chloro-5-[3-[[(4S)-1-[[3-[[4-[3-(2,6-dioxo-3-piperidyl)-1-ethyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylic acid (200)

3-(carboxymethoxy)-4-chloro-5-[3-[[(4S)-1-[[3-[4-[3-(2,6-dioxo-3-piperidyl)-1-ethyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylic acid (200, 80 mg, 69.72 μmol, 64% yield, TFA salt) was synthesized from tert-butyl 3-(2-tert-butoxy-2-oxo-ethoxy)-4-chloro-5-[3-[[(4S)-1-[[3-[[4-[3-(2,6-dioxo-3-piperidyl)-1-ethyl-indazol-6-yl]-3,3-difluoro-piperidine-1-carbonyl]amino]phenyl]methylsulfonyl]-2,2-dimethyl-4-piperidyl]amino]phenyl]thiophene-2-carboxylate (12, 230 mg, 109.47 μmol, HCl salt) in a similar fashion to Compound A-26. The residue was purified by reverse phase prep HPLC [Purification method: Column: X-select C18 (19×250) mm, 5 micron; Mobile phase A: 0.1% TFA in water; Mobile phase B: MeCN.

LCMS (ES+): m/z 1010.0 [M+H]⁺

¹HNMR (400 MHz, DMSO-d6): δ 10.90 (s, 1H), 8.82 (s, 1H), 7.66 (d, J=8.40 Hz, 1H), 7.59 (s, 2H), 7.55-7.50 (m, 3H), 7.30-7.26 (m, 1H), 7.20 (t, J=8.40 Hz, 1H), 7.09 (d, J=8.40 Hz, 1H), 7.03 (d, J=7.20 Hz, 1H), 6.89 (s, 1H), 6.85-6.81 (m, 1H), 6.71 (d, J=8.40 Hz, 1H), 4.92 (s, 2H), 4.65-4.55 (m, 1H), 4.41-4.26 (m, 6H), 3.57-3.30 (m, 3H), 3.14-3.09 (m, 2H), 2.68-672.00 (m, 2H), 2.34-2.08 (m, 3H), 1.95-1.79 (m, 3H), 1.51-1.37 (m, 12H), 1.15-1.08 (m, 1H).

### Example 201 and Example 202

### 3-(carboxymethoxy)-4-chloro-5-[3-[[(4S)-1-[[3-[2-[3-[[1-(2,6-dioxo-3-piperidyl)-2-oxo-benzo[cd]indol-6-yl]propylamino]ethylamino]phenyl]methylsulfonyl]-2,2-dimethyl-4-piperidyl]amino]phenyl]thiophene-2-carboxylic acid (201), and 3-(carboxymethoxy)-4-chloro-5-[3-[[(4S)-1-[[3-[3-[3-[[1-(2,6-dioxo-3-piperidyl)-2-oxo-benzo[cd]indol-6-yl]propyl]-2-oxo-imidazolidin-1-yl]phenyl]methylsulfonyl]-2,2-dimethyl-4-piperidyl]amino]phenyl]thiophene-2-carboxylic acid (202)

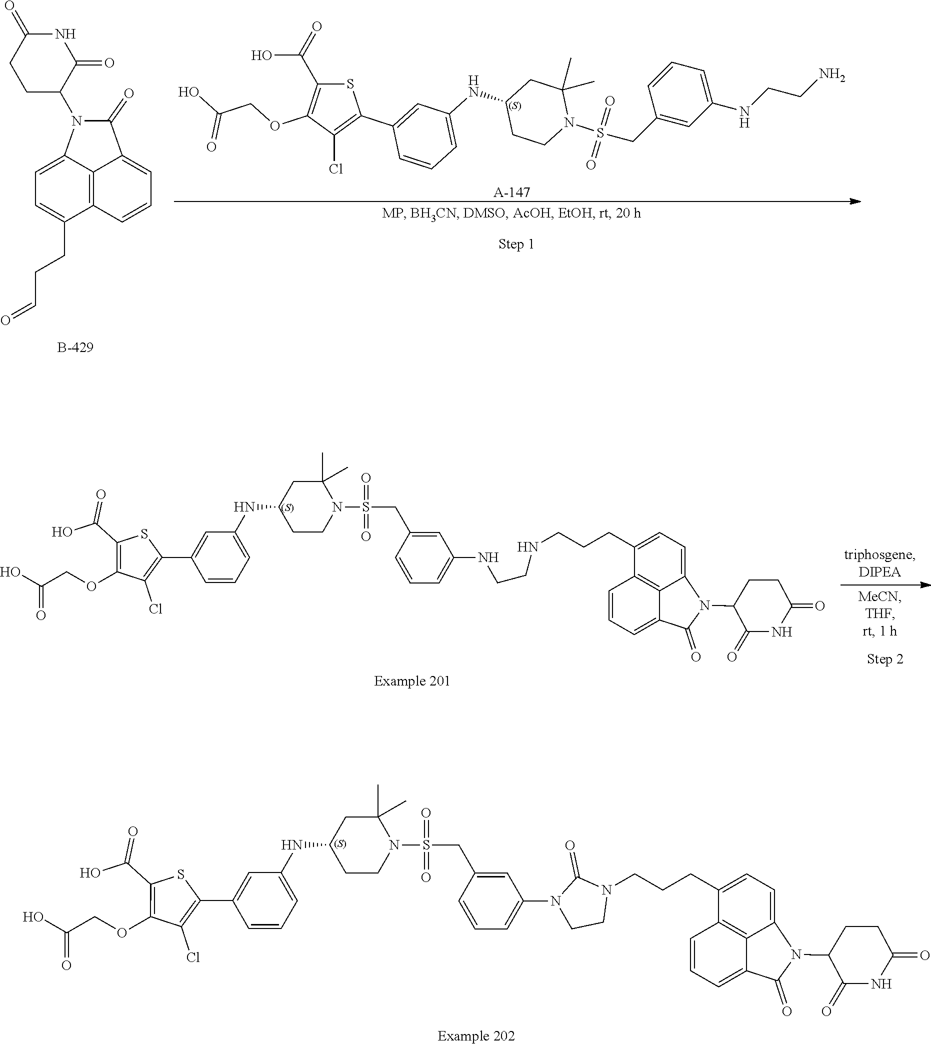

### Step 1: 3-(carboxymethoxy)-4-chloro-5-[3-[[(4S)-1-[3-[2-[3-[[1-(2,6-dioxo-3-piperidyl)-2-oxo-benzo[cd]indol-6-yl]propylamino]ethylamino]phenyl]methylsulfonyl]-2,2-dimethyl-4-piperidyl]amino]phenyl]thiophene-2-carboxylic acid (201)

3-(carboxymethoxy)-4-chloro-5-[3-[[(4S)-1-[[3-[2-[3-[[1-(2,6-dioxo-3-piperidyl)-2-oxo-benzo[cd]indol-6-yl]propylamino]ethylamino]phenyl]methylsulfonyl]-2,2-dimethyl-4-piperidyl]amino]phenyl]thiophene-2-carboxylic acid (201, 65 mg, 56.28 μmol, 6% yield, TFA salt) was synthesized from 3-[1-(2,6-dioxo-3-piperidyl)-2-oxo-benzo[cd]indol-6-yl]propanal (B-429, 500 mg, 903.85 μmol) and 5-[3-[[(4S)-1-[[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147, 922.18 mg, 903.85 μmol, TFA salt in a similar fashion to Compound A-74a/b, except using EtOH/DMSO as the solvent and including excess NaOAc. The residue purified by reverse-phase preparative HPLC [Column: Sunfire C18 (19×150 mm) 5 μm; Mobile phase A: 0.1% TFA in water and Mobile phase B: MeCN]. ¹H-NMR (400 MHz, DMSO-d6): δ 11.13 (s, 1H), 8.39-8.37 (m, 2H), 8.13 (d, J=5.60 Hz, 1H), 7.89 (d, J=8.00 Hz, 1H), 7.35 (d, J=6.80 Hz, 1H), 7.20 (t, J=8.00 Hz, 1H), 7.15-7.08 (m, 2H), 6.88 (s, 1H), 6.81 (d, J=8.00 Hz, 1H), 6.70-6.59 (m, 3H), 5.79-5.77 (m, 1H), 5.46-5.44 (m, 1H), 4.92 (s, 2H), 4.32-4.18 (m, 2H), 3.11-2.68 (m, 12H), 2.09-1.78 (m, 6H), 1.48-1.39 (m, 7H), 1.18-1.07 (m, 2H). LCMS (ES−): m/z 969.0 [M−H]⁺

### Step 2: 3-(carboxymethoxy)-4-chloro-5-[3-[[(4S)-1-[[3-[3-[3-[[1-(2,6-dioxo-3-piperidyl)-2-oxo-benzo[cd]indol-6-yl]propyl]-2-oxo-imidazolidin-1-yl]phenyl]methylsulfonyl]-2,2-dimethyl-4-piperidyl]amino]phenyl]thiophene-2-carboxylic acid (202)

3-(carboxymethoxy)-4-chloro-5-[3-[[(4S)-1-[[3-[3-[3-[[1-(2,6-dioxo-3-piperidyl)-2-oxo-benzo[cd]indol-6-yl]propyl]-2-oxo-imidazolidin-1-yl]phenyl]methylsulfonyl]-2,2-dimethyl-4-piperidyl]amino]phenyl]thiophene-2-carboxylic acid (202, 5.5 mg, 4.96 μmol, 9% yield, formic acid salt) was synthesized from 3-(carboxymethoxy)-4-chloro-5-[3-[[(4S)-1-[[3-[2-[3-[[1-(2,6-dioxo-3-piperidyl)-2-oxo-benzo[cd]indol-6-yl]propylamino]ethylamino]phenyl]methylsulfonyl]-2,2-dimethyl-4-piperidyl]amino]phenyl]thiophene-2-carboxylic acid (201, 65 mg, 56.28 μmol, TFA salt) in a similar fashion to Example 142, except using THE/Acetonitrile as the solvent. The residue was purified by reverse-phase preparative HPLC [Column: XBRIDGE OBD C18 19×150 mm, 5 micron; Mobile Phase A: 0.1% Formic acid in water and Mobile Phase B: MeCN]. ¹H-NMR (400 MHz, DMSO-d6): δ 11.13 (s, 1H), 8.36 (d, J=8.40 Hz, 1H), 8.09 (d, J=6.80 Hz, 1H), 7.84 (t, J=7.60 Hz, 1H), 7.64 (s, 1H), 7.55 (d, J=8.40 Hz, 1H), 7.38 (d, J=7.20 Hz, 1H), 7.32 (t, J=7.60 Hz, 1H), 7.17 (t, J=8.00 Hz, 1H), 7.07-7.04 (m, 2H), 6.84 (s, 1H), 6.81 (d, J=18.40 Hz, 1H), 6.66 (d, J=8.00 Hz, 1H), 5.43 (dd, J=5.20, 12.00 Hz, 1H), 4.82 (s, 2H), 4.43-4.28 (m, 2H), 3.80 (t, J=8.40 Hz, 2H), 3.51-3.34 (m, 4H), 3.17-2.95 (m, 7H), 2.08-1.76 (m, 6H), 1.45-1.36 (m, 7H), 1.24-1.10 (m, 2H). LCMS (ES+): m/z 996.7 [M+H]⁺.

### Example 203

### 3-(carboxymethoxy)-4-chloro-5-[3-[[(4S)-1-[[3-[[3-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (203)

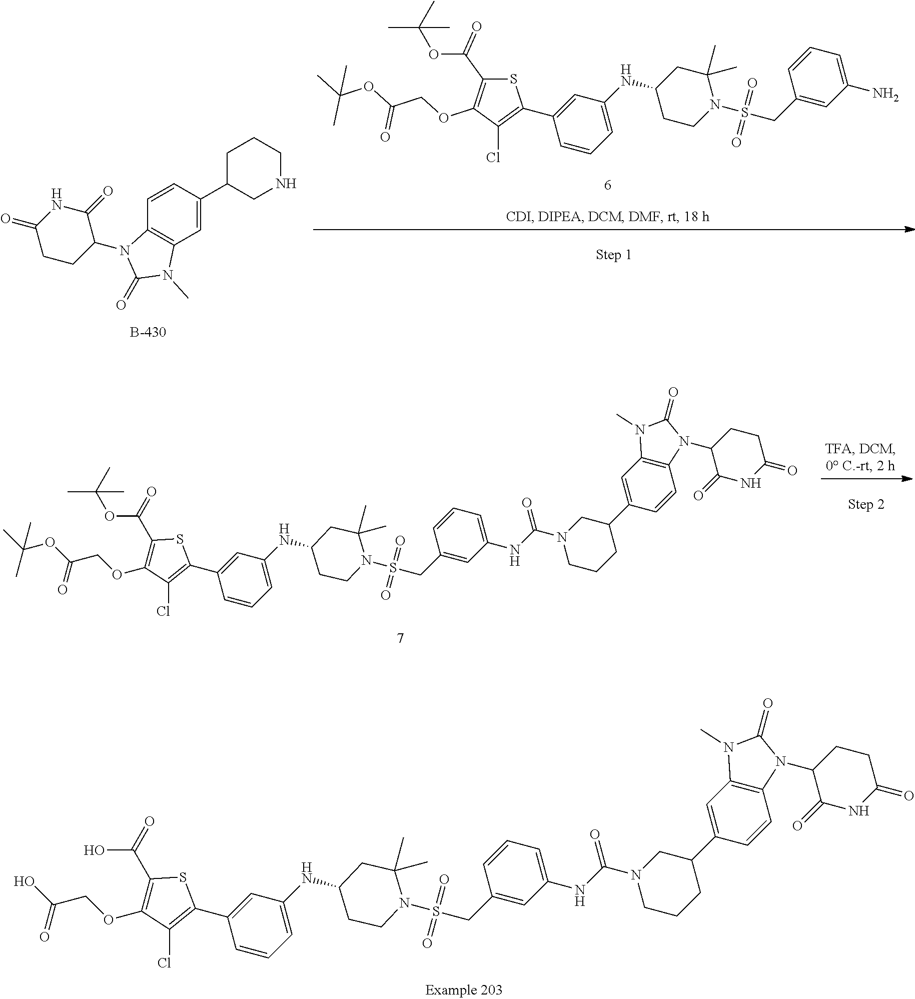 [SMILES: Cn1c(=O)n(C2CCC(=O)NC2=O)c2ccc(C3CCCNC3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(C3CCCN(C(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)C3)cc21 ; Cn1c(=O)n(C2CCC(=O)NC2=O)c2ccc(C3CCCN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21]

### Step 1: tert-butyl 3-(2-tert-butoxy-2-oxo-ethoxy)-4-chloro-5-[3-[[(4S)-1-[[3-[[3-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (7)

tert-butyl 3-(2-tert-butoxy-2-oxo-ethoxy)-4-chloro-5-[3-[[(4S)-1-[3-[3-[[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (7, 520 mg, 162.39 μmol, 68% yield, 34% purity) was from synthesized tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (6, 175 mg, 240.51 μmol) and 3-[3-methyl-2-oxo-5-(3-piperidyl)benzimidazol-1-yl]piperidine-2,6-dione (B-430, 113.17 mg, 240.51 μmol, TFA salt) in a similar fashion to Compound B-242. LCMS (ES+): m/z 1088.2 [M+H]⁺

### Step 2: 3-(carboxymethoxy)-4-chloro-5-[3-[[(4S)-1-[[3-[[3-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (203)

3-(carboxymethoxy)-4-chloro-5-[3-[[(4S)-1-[3-[3-[[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (203, 81 mg, 72.96 μmol, 45% yield, TFA salt) was synthesized from tert-butyl 3-(2-tert-butoxy-2-oxo-ethoxy)-4-chloro-5-[3-[[(4S)-1-[[3-[[3-[1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (7, 520 mg, 162.39 μmol) in a similar fashion to Compound A-26. The residue was purified by Reverse Phase Prep HPLC [Column: XSelect C18 (19×250) mm, 5 micron; Mobile Phase A: 0.1% TFA in water; Mobile phase B: MeCN]. LCMS (ES+): m/z 976.2 [M+H]⁺

¹HNMR (400 MHz, DMSO-d6): δ 13.20 (bs, 1H), 11.09 (s, 1H), 8.60 (s, 1H), 7.58 (s, 1H), 7.44 (d, J=8.40 Hz, 1H), 7.26-7.16 (m, 3H), 7.05 (d, J=7.60 Hz, 1H), 7.00-6.98 (m, 2H), 6.87 (s, 1H), 6.81 (d, J=7.20 Hz, 1H), 6.67 (d, J=7.60 Hz, 1H), 5.38-5.34 (m, 1H), 4.92 (s, 2H), 4.36 (d, J=13.60 Hz, 1H), 4.26-4.23 (m, 3H), 3.53-3.48 (m, 3H), 3.44 (s, 3H), 3.21 (t, J=12.80 Hz, 1H), 2.95-2.82 (m, 3H), 2.72-2.61 (m, 3H), 2.03-1.73 (m, 6H), 1.61-1.48 (m, 9H), 1.15-1.05 (m, 1H).

### Example 204

### 3-(carboxymethoxy)-4-chloro-5-(3-(((4S)-1-((3-(2-(4-(1-(2,6-dioxopiperidin-3-yl)-3-isopropyl-2-oxo-2,3-dihydro-1H-benzo[d]imidazol-5-yl)piperidin-1-yl)acetamido)benzyl)sulfonyl)-2,2-dimethylpiperidin-4-yl)amino)phenyl)thiophene-2-carboxylic acid (204)

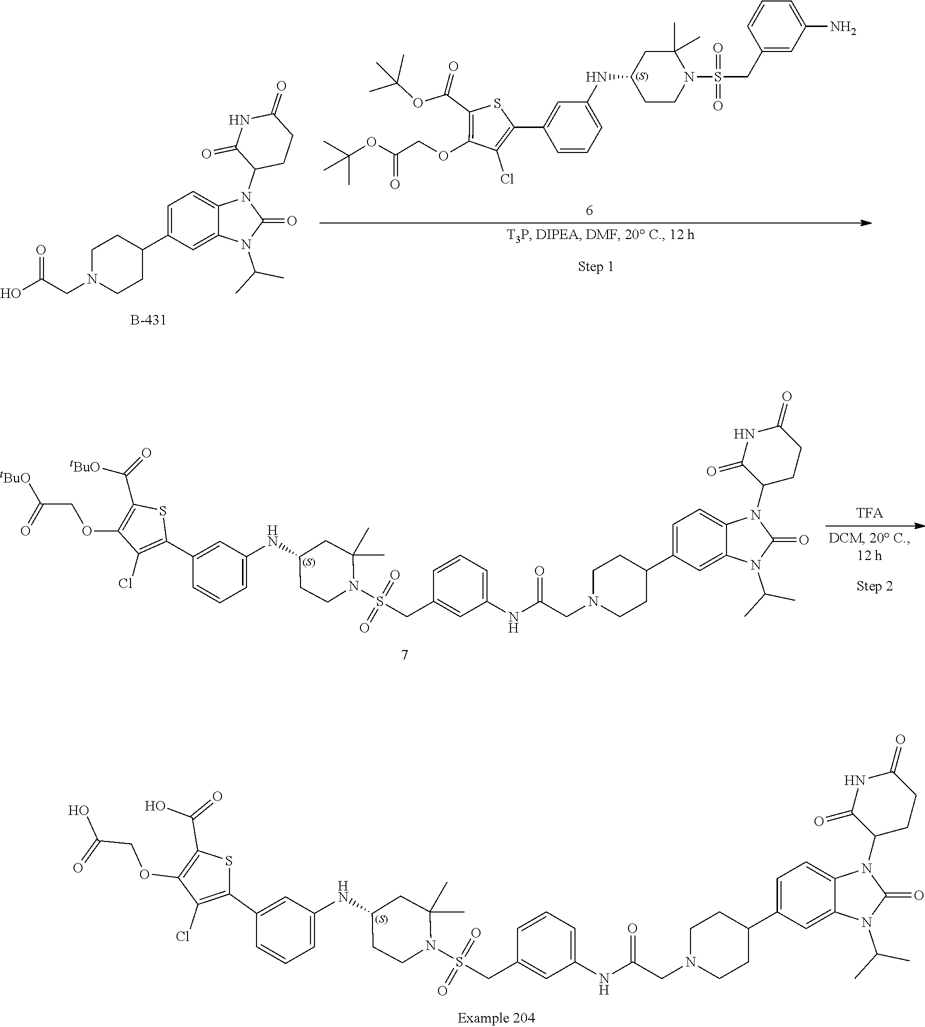

### Step 1: tert-butyl 3-(2-(tert-butoxy)-2-oxoethoxy)-4-chloro-5-(3-(((4S)-1-((3-(2-(4-(1-(2,6-dioxopiperidin-3-yl)-3-isopropyl-2-oxo-2,3-dihydro-1H-benzo[d]imidazol-5-yl)piperidin-1-yl)acetamido)benzyl)sulfonyl)-2,2-dimethylpiperidin-4-yl)amino)phenyl)thiophene-2-carboxylate (7)

tert-butyl 3-(2-(tert-butoxy)-2-oxoethoxy)-4-chloro-5-(3-(((4S)-1-((3-(2-(4-(1-(2,6-dioxopiperidin-3-yl)-3-isopropyl-2-oxo-2,3-dihydro-1H-benzo[d]imidazol-5-yl)piperidin-1-yl)acetamido)benzyl)sulfonyl)-2,2-dimethylpiperidin-4-yl)amino)phenyl)thiophene-2-carboxylate (7, 68 mg, 58.33 μmol, 55% yield) was synthesized from 2-(4-(1-(2,6-dioxopiperidin-3-yl)-3-isopropyl-2-oxo-2,3-dihydro-1H-benzo[d]imidazol-5-yl)piperidin-1-yl)acetic acid (B-431, 45 mg, 105.02 μmol) and tert-butyl(S)-tert-butyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (6, 75.65 mg, 105.02 μmol) in a similar fashion to Compound A-88. The residue was purified by prep-TLC (SiO2, Petroleum ether:Ethyl acetate=1:3).

LCMS (ES+): m/z 1130.5 [M+H]⁺

### Step 2: 3-(carboxymethoxy)-4-chloro-5-(3-(((4S)-1-((3-(2-(4-(1-(2,6-dioxopiperidin-3-yl)-3-isopropyl-2-oxo-2,3-dihydro-1H-benzo[d]imidazol-5-yl)piperidin-1-yl)acetamido)benzyl)sulfonyl)-2,2-dimethylpiperidin-4-yl)amino)phenyl)thiophene-2-carboxylic acid (204)

3-(carboxymethoxy)-4-chloro-5-(3-(((4S)-1-((3-(2-(4-(1-(2,6-dioxopiperidin-3-yl)-3-isopropyl-2-oxo-2,3-dihydro-1H-benzo[d]imidazol-5-yl)piperidin-1-yl)acetamido)benzyl)sulfonyl)-2,2-dimethylpiperidin-4-yl)amino)phenyl)thiophene-2-carboxylic acid (204, 13.4 mg, 13.02 μmol, 29% yield) was synthesized from tert-butyl 3-(2-(tert-butoxy)-2-oxoethoxy)-4-chloro-5-(3-(((4S)-1-((3-(2-(4-(1-(2,6-dioxopiperidin-3-yl)-3-isopropyl-2-oxo-2,3-dihydro-1H-benzo[d]imidazol-5-yl)piperidin-1-yl)acetamido)benzyl)sulfonyl)-2,2-dimethylpiperidin-4-yl)amino)phenyl)thiophene-2-carboxylate (7, 50.00 mg, 44.22 μmol) in a similar fashion to Compound A-26. The residue was purified by prep-HPLC (flow: 25 mL/min; gradient: from 10-55% MeCN in water (0.1% TFA); column: Phenomenex Synergi C18 150×25 mm×10 μm).

LCMS (ES+): m/z 1018.3 [M+H]⁺

¹H NMR (400 MHz, DMSO-d₆) δ 11.08 (s, 1H), 10.54-9.86 (m, 1H), 7.75 (s, 1H), 7.64 (d, J=8.8 Hz, 1H), 7.41-7.31 (m, 1H), 7.24-7.10 (m, 3H), 7.03 (d, J=8.0 Hz, 1H), 6.91 (d, J=7.6 Hz, 1H), 6.84 (s, 1H), 6.78 (d, J=7.6 Hz, 1H), 6.66 (d, J=7.6 Hz, 1H), 5.72-5.61 (m, 1H), 5.33 (d, J=12.8 Hz, 1H), 4.67 (s, 2H), 4.64-4.56 (m, 1H), 4.44 (d, J=13.6 Hz, 1H), 4.38-4.29 (m, 1H), 3.51-3.50 (m, 2H), 3.48-3.42 (m, 4H), 3.13 (d, J=4.8 Hz, 1H), 2.91 (d, J=4.8 Hz, 1H), 2.74-2.63 (m, 4H), 2.03-1.80 (m, 8H), 1.47 (m, 9H), 1.39 (s, 4H), 1.09-1.04 (m, 1H).

### Example 205

### 3-(carboxymethoxy)-4-chloro-5-[3-[[(4S)-1-[[3-[[4-[1-(2,6-dioxo-3-piperidyl)-2-oxo-benzo[cd]indol-S-yl]piperidine-1-carbonyl]amino]phenyl]methylsulfonyl]-2,2-dimethyl-4-piperidyl]amino]phenyl]thiophene-2-carboxylic acid (205)

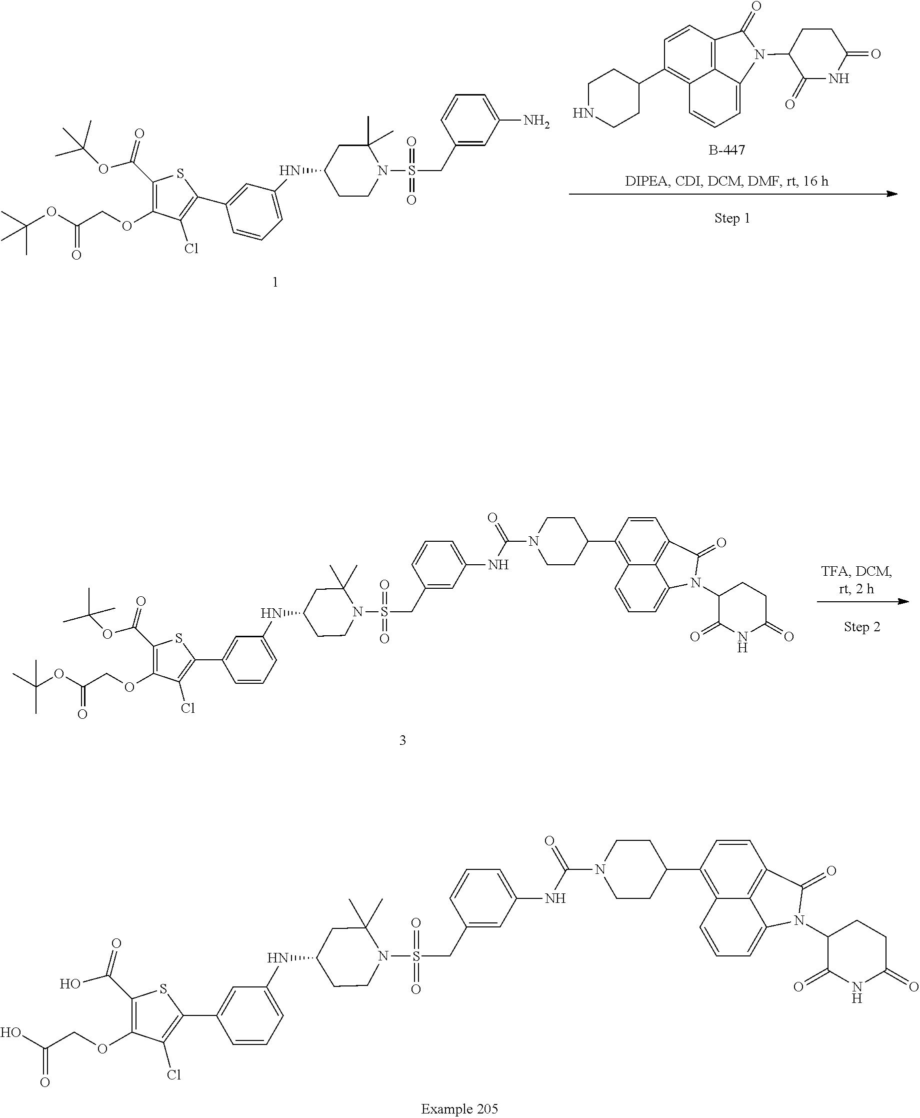 [SMILES: CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; O=C1CCC(N2C(=O)c3ccc(C4CCNCC4)c4cccc2c34)C(=O)N1 ; CC(C)(C)OC(=O)COc1c(C(=O)OC(C)(C)C)sc(-c2cccc(N[C@H]3CCN(S(=O)(=O)Cc4cccc(NC(=O)N5CCC(c6ccc7c8c(cccc68)N(C6CCC(=O)NC6=O)C7=O)CC5)c4)C(C)(C)C3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)N(C3CCC(=O)NC3=O)C4=O)CC2)c1]

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-5-yl]piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-5-yl]piperidine-1-carbonyl]amino]phenyl]methylsulfonyl]-2,2-dimethyl-4-piperidyl]amino]phenyl]thiophene-2-carboxy late (3, 160 mg, 74.23 μmol, 36% yield, 53% purity, HCl salt) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (1, 150 mg, 206.15 μmol and 3-[2-oxo-5-(4-piperidyl)benzo[cd]indol-1-yl]piperidine-2,6-dione (B-447, 74.92 mg, 206.15 μmol) in a similar fashion to Compound B-242. The residue was suspended in aqueous 1.5 N HCl (30 mL). The solid was filtered and dried.

LCMS (ES+): m/z 1109.2 [M+H]⁺

### Step 2: 3-(carboxymethoxy)-4-chloro-S-[3-[[(4S)-1-[[3-[[4-[1-(2,6-dioxo-3-piperidyl)-2-oxo-benzo[cd]indol-5-yl]piperidine-1-carbonyl]amino]phenyl]methylsulfonyl]-2,2-dimethyl-4-piperidyl]amino]phenyl]thiophene-2-carboxylic acid (205)

3-(carboxymethoxy)-4-chloro-5-[3-[[(4S)-1-[3-[4-[1-(2,6-dioxo-3-piperidyl)-2-oxo-benzo[cd]indol-5-yl]piperidine-1-carbonyl]amino]phenyl]methylsulfonyl]-2,2-dimethyl-4-piperidyl]amino]phenyl]thiophene-2-carboxylic acid (205, 60 mg, 51.46 μmol, 70% yield, TFA salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-2-oxo-benzo[cd]indol-5-yl]piperidine-1-carbonyl]amino]phenyl]methylsulfonyl]-2,2-dimethyl-4-piperidyl]amino]phenyl]thiophene-2-carboxylate (3, 160 mg, 73.98 μmol, HCl salt) in a similar fashion to Compound A-26. The residue was purified by reverse phase column chromatography [Purification method: Silicycle C18 column; Mobile phase A: 0.1% TFA in water; Mobile phase B: MeCN].

LCMS (ES+): m/z 997.0 [M+H]⁺

¹HNMR (400 MHz, DMSO-d6): δ 13.01 (s, 1H), 11.12 (s, 1H), 8.69 (s, 1H), 8.04 (d, J=7.60 Hz, 1H), 7.92 (d, J=8.80 Hz, 1H), 7.72 (d, J=7.60 Hz, 1H), 7.59-7.55 (m, 2H), 7.49 (d, J=8.00 Hz, 1H), 7.28-7.16 (m, 3H), 7.00 (d, J=6.40 Hz, 1H), 6.88 (s, 1H), 6.81 (d, J=7.20 Hz, 1H), 6.70 (d, J=8.00 Hz, 1H), 5.50-5.44 (m, 1H), 4.91 (s, 2H), 4.39-4.25 (m, 2H), 3.72-3.33 (m, 4H), 3.31-2.93 (m, 5H), 2.82-2.64 (m, 3H), 2.15-2.09 (m, 1H), 1.94-1.77 (m, 6H), 1.50-1.40 (m, 7H), 1.13-1.10 (m, 1H).

### Example 206

### 3-(carboxymethoxy)-4-chloro-5-(3-(((4R)-1-((3-(4-(1-(2,6-dioxopiperidin-3-yl)-3-(oxetan-3-yl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (206)

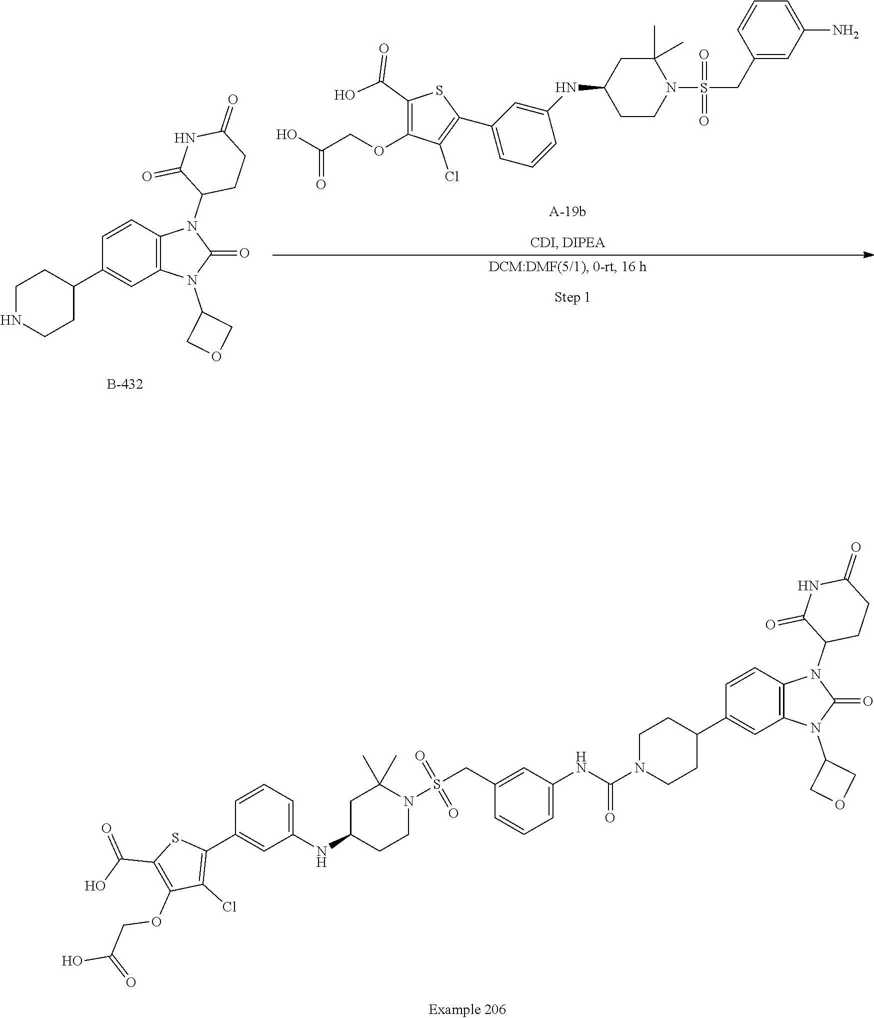 [SMILES: O=C1CCC(n2c(=O)n(C3COC3)c3cc(C4CCNCC4)ccc32)C(=O)N1 ; CC1(C)C[C@H](Nc2cccc(-c3sc(C(=O)O)c(OCC(=O)O)c3Cl)c2)CCN1S(=O)(=O)Cc1cccc(N)c1 ; CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C3COC3)c(=O)n4C3CCC(=O)NC3=O)CC2)c1]

### Step 1: 3-(carboxymethoxy)-4-chloro-5-(3-(((4R)-1-((3-(4-(1-(2,6-dioxopiperidin-3-yl)-3-(oxetan-3-yl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (206)

3-(carboxymethoxy)-4-chloro-5-(3-(((4R)-1-((3-(4-(1-(2,6-dioxopiperidin-3-yl)-3-(oxetan-3-yl)-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylic acid (206, 33.98 mg, 29.70 μmol, 10% yield, TFA salt) was synthesized from (R)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19b, 174.01 mg, 286.14 μmol) and 3-(3-(oxetan-3-yl)-2-oxo-5-(piperidin-4-yl)-2,3-dihydro-1H-benzo[d]imidazol-1-yl)piperidine-2,6-dione (B-432, reaction mixture) in a similar fashion to Compound B-242. The residue was purified by prep-HPLC (flow: 25 mL/min; gradient: from 42-62% MeCN-water (0.1% TFA) over 7 min; column: 3_Phenomenex Luna C18 75×30 mm×3 μm).

LCMS (ESI): m/z 1018.5 [M+H]⁺

¹H NMR (400 MHz, DMSO-d₆) δ 11.10 (s, 1H), 8.62 (s, 1H), 7.57 (s, 1H), 7.48-7.42 (m, 2H), 7.26-7.17 (m, 2H), 7.08 (d, J=8.4 Hz, 1H), 7.01-6.96 (m, 2H), 6.87 (s, 1H), 6.81 (d, J=7.6 Hz, 1H), 6.73-6.65 (m, 1H), 5.57-5.50 (m, 1H), 5.32-5.32 (m, 1H), 5.11 (t, J=2.0, 6.4 Hz, 2H), 4.95 (t, J=7.2 Hz, 2H), 4.90 (s, 2H), 4.38-4.25 (m, 4H), 3.48-3.43 (m, 2H), 3.12 (t, J=12.0 Hz, 1H), 2.95-2.86 (m, 3H), 2.81-2.64 (s, 3H), 2.04-1.98 (m, 1H), 1.92-1.73 (m, 4H), 1.66-1.58 (m, 2H), 1.48 (s, 4H), 1.39 (s, 3H), 1.17-1.03 (m, 1H).

### Example 207

### 3-(carboxymethoxy)-4-chloro-5-[3-[[(4S)-1-[3-[4-[4-chloro-1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (207)

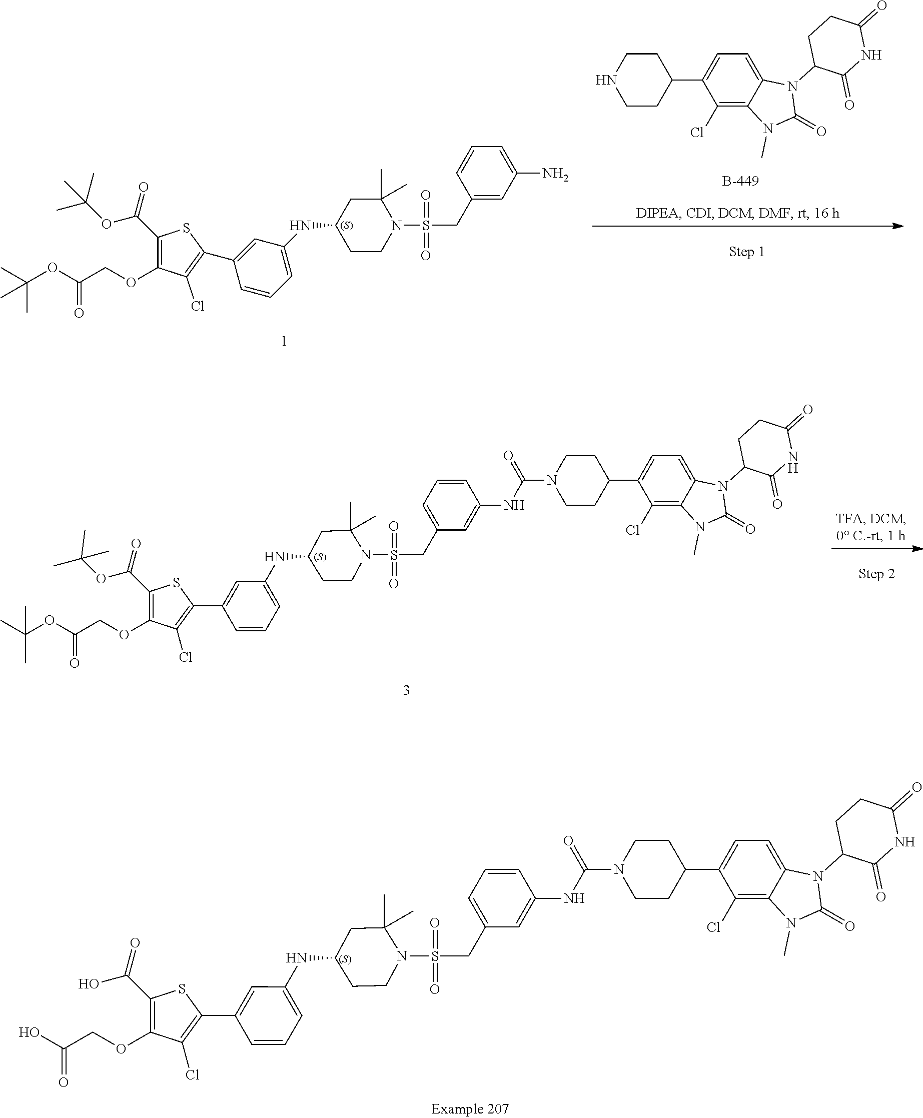

### Step 1: tert-butyl 3-(2-tert-butoxy-2-oxo-ethoxy)-4-chloro-5-[3-[[(4S)-1-[[3-[[4-[4-chloro-1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[[3-[[4-[4-chloro-1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (3, 231 mg, 107.99 μmol, 46% yield, 54% purity, HCl salt) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxy late (1, 170 mg, 233.64 μmol) and 3-[4-chloro-3-methyl-2-oxo-5-(4-piperidyl)benzimidazol-1-yl]piperidine-2,6-dione (B-449, 137.62 mg, 280.37 μmol, TFA salt) in a similar fashion to Compound B-242. The residue was suspended in aqueous 1.5 N HCl (30 mL) solution. The solid was filtered to afford the product. LCMS (ES+): m/z 1122.0 [M+H]⁺

### Step 2: 3-(carboxymethoxy)-4-chloro-5-[3-[[(4S)-1-[[3-[[4-[4-chloro-1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (207)

3-(carboxymethoxy)-4-chloro-5-[3-[[(4S)-1-[3-[4-[4-chloro-1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (207, 70 mg, 59.85 μmol, 56% yield, TFA salt) was synthesized from tert-butyl 3-(2-tert-butoxy-2-oxo-ethoxy)-4-chloro-5-[3-[[(4S)-1-[[3-[[4-[4-chloro-1-(2,6-dioxo-3-piperidyl)-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (3, 230 mg, 107.10 μmol, HCl salt) in a similar fashion to Compound A-26. The residue was purified by reverse phase column chromatography [Purification method: Silicycle C18 column; Mobile phase A: 0.1% TFA in water; Mobile phase B: MeCN]. LCMS (ES+): m/z 1012.0 [M+H]⁺

¹HNMR (400 MHz, DMSO-d6): δ 11.14 (s, 1H), 8.65 (s, 1H), 7.58 (s, 1H), 7.48-7.46 (m, 1H), 7.27-7.19 (m, 2H), 7.11-7.05 (m, 2H), 6.99 (d, J=8.00 Hz, 1H), 6.88 (s, 1H), 6.82 (d, J=8.00 Hz, 1H), 6.70 (dd, J=1.20, 8.20 Hz, 1H), 5.42-5.37 (m, 1H), 4.91 (s, 2H), 4.39-4.24 (m, 4H), 3.67 (s, 3H), 3.65-3.44 (m, 2H), 3.28-3.06 (m, 2H), 2.97-2.90 (m, 3H), 2.71-2.65 (m, 2H), 2.15-1.98 (m, 1H), 1.84-1.77 (m, 4H), 1.60-1.52 (m, 2H), 1.49 (s, 3H), 1.40 (s, 3H), 1.25-1.11 (m, 1H).

### Example 208

### 3-(carboxymethoxy)-4-chloro-5-[3-[[(4S)-1-[[3-[[4-[1-(2,6-dioxo-3-piperidyl)-2-methyl-indol-5-yl]piperidine-1-carbonyl]amino]phenyl]methylsulfonyl]-2,2-dimethyl-4-piperidyl]amino]phenyl]thiophene-2-carboxylic acid (208)

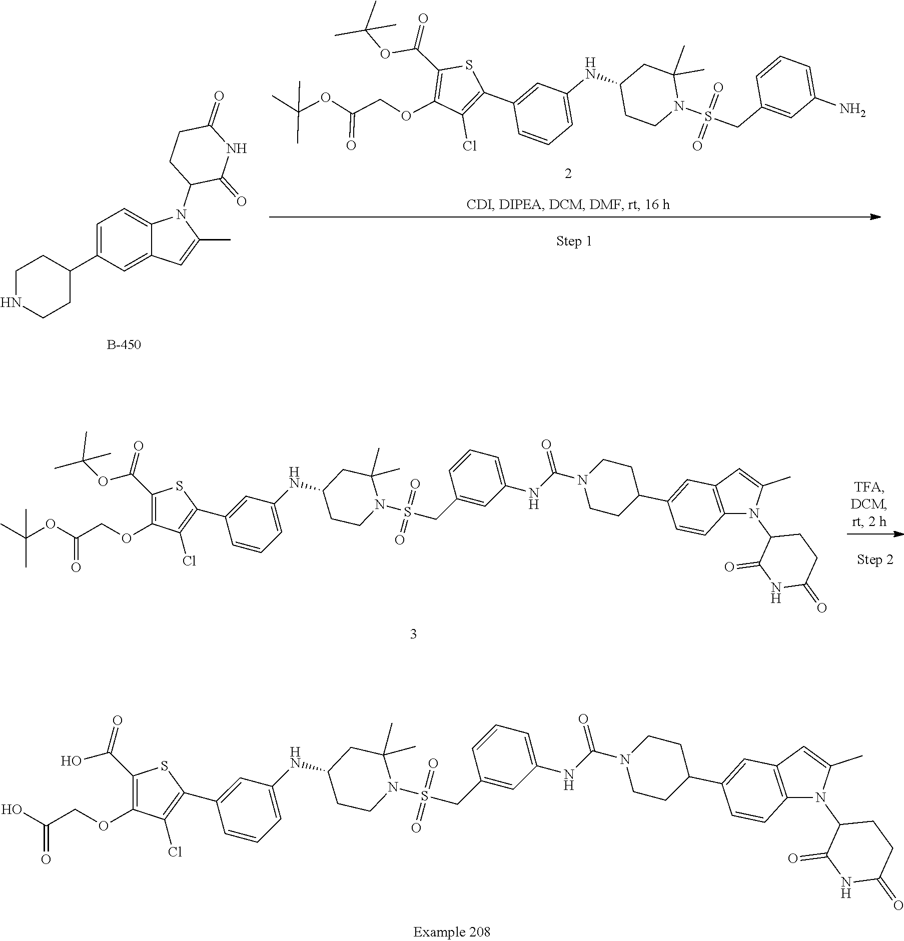 [SMILES: Cc1cc2cc(C3CCNCC3)ccc2n1C1CCC(=O)NC1=O ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cc1cc2cc(C3CCN(C(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3)ccc2n1C1CCC(=O)NC1=O ; Cc1cc2cc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)ccc2n1C1CCC(=O)NC1=O]

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-2-methyl-indol-5-yl]piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[3-[4-[1-(2,6-dioxo-3-piperidyl)-2-methyl-indol-5-yl]piperidine-1-carbonyl]amino]phenyl]methylsulfonyl]-2,2-dimethyl-4-piperidyl]amino]phenyl]thiophene-2-carboxylate (3, 65 mg, 38.82 μmol, 71% yield, 64% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2, 40 mg, 54.97 μmol) and 3-[2-methyl-5-(4-piperidyl) indol-1-yl]piperidine-2,6-dione (B-450, 34.40 mg, 54.97 μmol) in a similar fashion to Compound B-242. After addition of water to the reaction, the solid was filtered off and dried under reduced pressure.

LCMS (ES+): m/z 1071.2 [M+H]⁺

### Step 2: 3-(carboxymethoxy)-4-chloro-5-[3-[[(4S)-1-[[3-[[4-[1-(2,6-dioxo-3-piperidyl)-2-methyl-indol-5-yl]piperidine-1-carbonyl]amino]phenyl]methylsulfonyl]-2,2-dimethyl-4-piperidyl]amino]phenyl]thiophene-2-carboxylic acid (208)

3-(carboxymethoxy)-4-chloro-5-[3-[[(4S)-1-[[3-[[4-[1-(2,6-dioxo-3-piperidyl)-2-methyl-indol-5-yl]piperidine-1-carbonyl]amino]phenyl]methylsulfonyl]-2,2-dimethyl-4-piperidyl]amino]phenyl]thiophene-2-carboxylic acid (208, 10 mg, 9.54 μmol, 32% yield, Formic acid salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-2-methyl-indol-5-yl]piperidine-1-carbonyl]amino]phenyl]methylsulfonyl]-2,2-dimethyl-4-piperidyl]amino]phenyl]thiophene-2-carboxylate (3, 50 mg, 29.86 μmol) in a similar fashion to Compound A-26. The residue was purified by reverse phase prep HPLC [Purification method: Column: Xbridge C18 (20×150) mm, Mobile phase A: 0.1% Formic acid in water; Mobile phase B: Acetonitrile].

LCMS (ES+): m/z 958.7 [M+H]⁺

¹HNMR (400 MHz, DMSO-d6): δ 11.03 (s, 1H), 8.62 (s, 1H), 7.59 (s, 1H), 7.48 (d, J=9.20 Hz, 1H), 7.35-7.17 (m, 4H), 7.09 (d, J=0.80 Hz, 1H), 7.04 (d, J=10.00 Hz, 1H), 6.99 (d, J=7.60 Hz, 1H), 6.86 (s, 1H), 6.80 (d, J=7.60 Hz, 1H), 6.68 (d, J=8.00 Hz, 1H), 5.72-5.62 (m, 1H), 5.55-5.45 (m, 1H), 4.83 (s, 2H), 4.39-4.24 (m, 4H), 3.49-3.33 (m, 2H), 3.17-3.08 (m, 2H), 2.95-2.89 (m, 4H), 2.69-2.64 (m, 3H), 2.23 (s, 3H), 2.12-2.08 (m, 1H), 1.91-1.75 (m, 4H), 1.71-1.59 (m, 2H), 1.50-1.40 (m, 7H), 1.15-1.05 (m, 1H).

### Example 209

### 3-(carboxymethoxy)-4-chloro-5-[3-[[(4S)-1-[[3-[(3R)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylic acid (209)

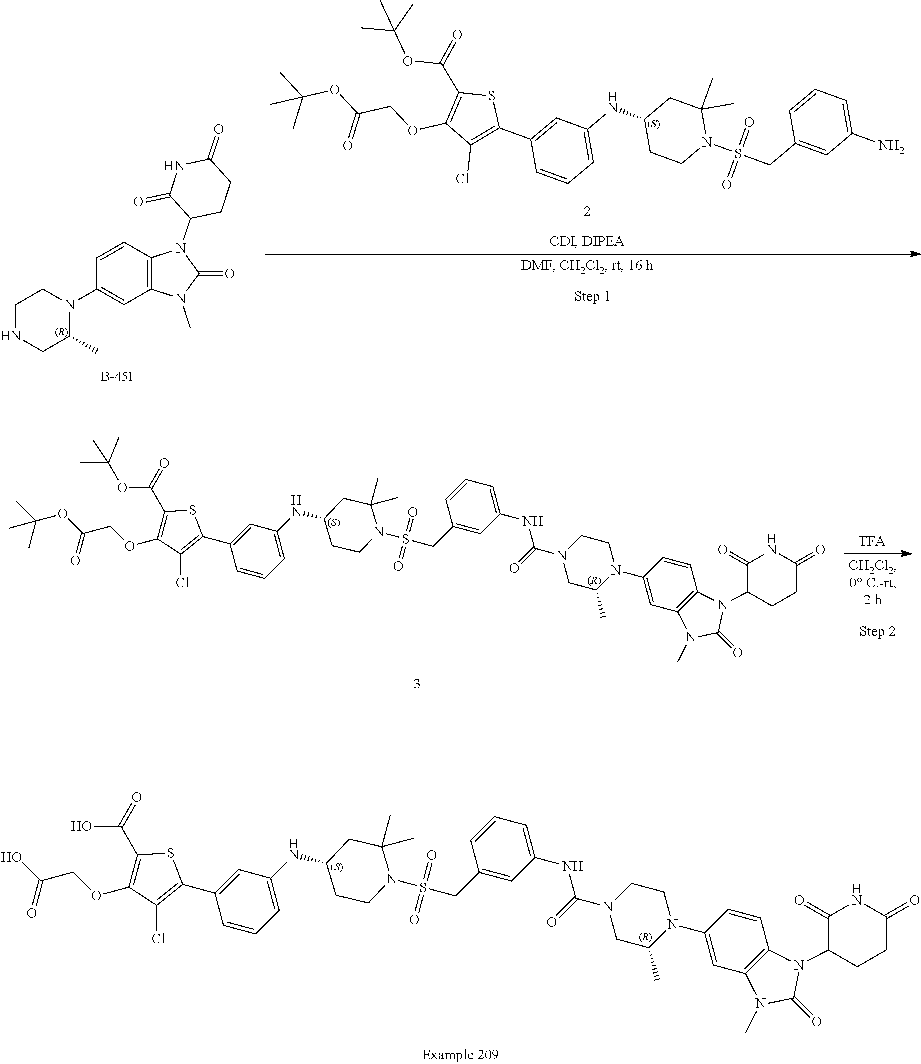

### Step 1: tert-butyl 3-(2-tert-butoxy-2-oxo-ethoxy)-4-chloro-5-[3-[[(4S)-1-[[3-[(3R)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[3-[(3R)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylate (3, 139 mg, 60.05 μmol, 32% yield, 48% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2, 135 mg, 187.41 μmol) and 3-[3-methyl-5-[(2R)-2-methylpiperazin-1-yl]-2-oxo-benzimidazol-1-yl]piperidine-2,6-dione (B-451, 80.38 mg, 224.89 μmol) in a similar fashion to Compound B-242. The reaction mixture was concentrated under reduced pressure to afford the residue which was diluted with 1.0 mL water to precipitate a solid that was filtered, washed with water and dried under vacuum to give product.

LCMS (ES+): m/z 1103.2

### Step 2: 3-(carboxymethoxy)-4-chloro-5-[3-[[(4S)-1-[[3-[[(3R)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylic acid (209)

3-(carboxymethoxy)-4-chloro-5-[3-[[(4S)-1-[3-[(3R)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylic acid (209, 35 mg, 31.47 μmol, 46% yield, TFA salt) was synthesized from tert-butyl 3-(2-tert-butoxy-2-oxo-ethoxy)-4-chloro-5-[3-[[(4S)-1-[[3-[(3R)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylate (3, 159 mg, 68.69 μmol) in a similar fashion to Compound A-26. The residue was purified by reverse-phase preparative HPLC [X-Select C18 (150×19.1) mm, 5.0 μm; Mobile Phase A: 0.1% TFA in water and Mobile Phase B: Acetonitrile].

¹H-NMR (400 MHz, DMSO-d6): δ 11.11 (s, 1H), 8.75 (s, 1H), 7.57 (s, 1H), 7.50 (d, J=8.80 Hz, 1H), 7.28 (t, J=8.00 Hz, 1H), 7.21 (t, J=8.00 Hz, 1H), 7.09-7.02 (m, 3H), 6.88 (s, 1H), 6.82 (d, J=8.40 Hz, 1H), 6.70 (d, J=9.60 Hz, 1H), 5.37-5.35 (m, 1H), 4.92 (s, 2H), 4.40-4.26 (m, 2H), 3.89-3.54 (m, 6H), 3.50 (s, 3H), 3.18-3.14 (m, 2H), 2.95-2.82 (m, 2H), 2.68-2.56 (m, 4H), 2.10-1.99 (m, 1H), 1.96-1.75 (m, 2H), 1.78 (s, 3H), 1.49 (m, 3H), 1.20-1.05 (m, 1H), 0.95-0.94 (m, 2H). LCMS (ES+): m/z 993.0 [M+H]⁺.

### Example 210

### 3-(carboxymethoxy)-4-chloro-5-[3-[[(4S)-1-[[3-[(3S)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylic acid (210)

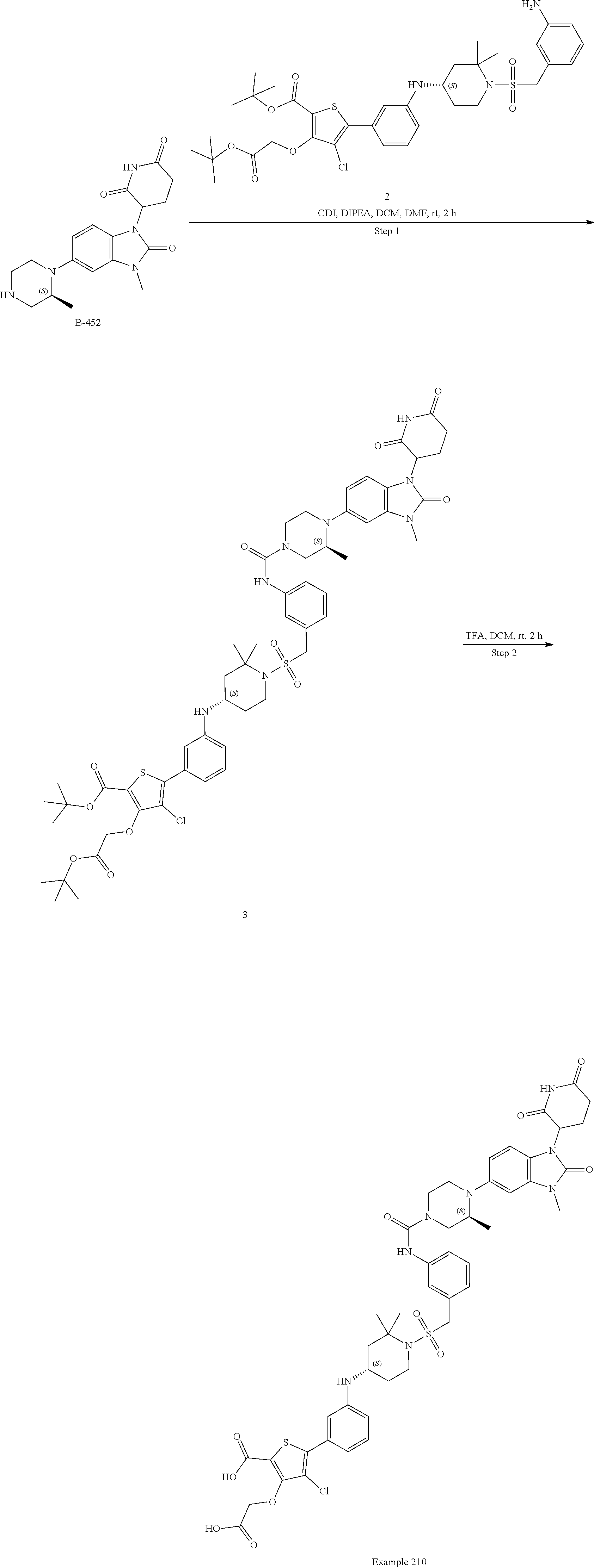

### Step 1: tert-butyl 3-(2-tert-butoxy-2-oxo-ethoxy)-4-chloro-5-[3-[[(4S)-1-[[3-[[(3S)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[[3-[(3S)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylate (180 mg, 135.20 μmol, 65% yield) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (150 mg, 206.99 μmol) and 3-[3-methyl-5-[(2S)-2-methylpiperazin-1-yl]-2-oxo-benzimidazol-1-yl]piperidine-2,6-dione (B-452, 77.87 mg, 206.99 μmol) in a similar fashion to Compound B-242. The reaction mixture was concentrated and the residue was triturated with water (2 mL) and the solid was filtered, washed with water and dried to afford product.

LCMS (ES+): m/z 1103.2 [M+H]⁺

### Step 2: 3-(carboxymethoxy)-4-chloro-5-[3-[[(4S)-1-[3-[(3S)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylic acid (210)

3-(carboxymethoxy)-4-chloro-5-[3-[[(4S)-1-[3-[(3S)-4-[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylic acid (210, 103.08 mg, 90.86 μmol, 64% yield, TFA salt) was synthesized from tert-butyl 3-(2-tert-butoxy-2-oxo-ethoxy)-4-chloro-5-[3-[[(4S)-1-[[3-[[(3S)-4-[[1-(2,6-dioxo-3-piperidyl)-3-methyl-2-oxo-benzimidazol-5-yl]-3-methyl-piperazine-1-carbonyl]amino]phenyl]methylsulfonyl]-2,2-dimethyl-4-piperidyl]amino]phenyl]thiophene-2-carboxylate (190 mg, 142.71 μmol) in a similar fashion to Compound A-26. The residue was purified with reverse phase preparatory HPLC (Column: XSelect C18 (250×19) mm, 5 micron; Mobile phase: A: 0.1% TFA in water, B: Acetonitrile].

LCMS (ES+): m/z 992.0 [M+H]⁺

¹HNMR (400 MHz, DMSO-d6): δ 13.34 (bs, 1H), 11.11 (s, 1H), 8.85 (m, 2H), 7.57 (s, 1H), 7.50 (d, J=8.00 Hz, 1H), 7.28 (t, J=7.60 Hz, 1H), 7.20 (t, J=8.00 Hz, 1H), 7.10-7.04 (m, 1H), 7.02 (d, J=7.60 Hz, 1H), 6.88 (s, 1H), 6.81 (d, J=8.00 Hz, 1H), 6.70 (d, J=8.00 Hz, 1H), 5.45-5.35 (m, 1H), 4.92 (s, 1H), 4.33 (dd, J=14.00, 44.20 Hz, 2H), 3.81-3.54 (m, 3H), 3.54-3.45 (m, 3H), 3.34 (s, 3H), 3.17-3.13 (m, 2H), 2.95-2.85 (m, 1H), 2.71-2.61 (m, 2H), 2.04-2.01 (m, 1H), 1.88-1.78 (m, 2H), 1.50-1.40 (m, 7H), 1.17-1.05 (m, 1H), 0.94 (d, J=5.20 Hz, 3H).

### Example 211

### 3-(carboxymethoxy)-4-chloro-5-[3-[[(4S)-1-[[3-[[4-[1-(2,6-dioxo-3-piperidyl)-3-methyl-indol-5-yl]piperidine-1-carbonyl]amino]phenyl]methylsulfonyl]-2,2-dimethyl-4-piperidyl]amino]phenyl]thiophene-2-carboxylic acid (211)

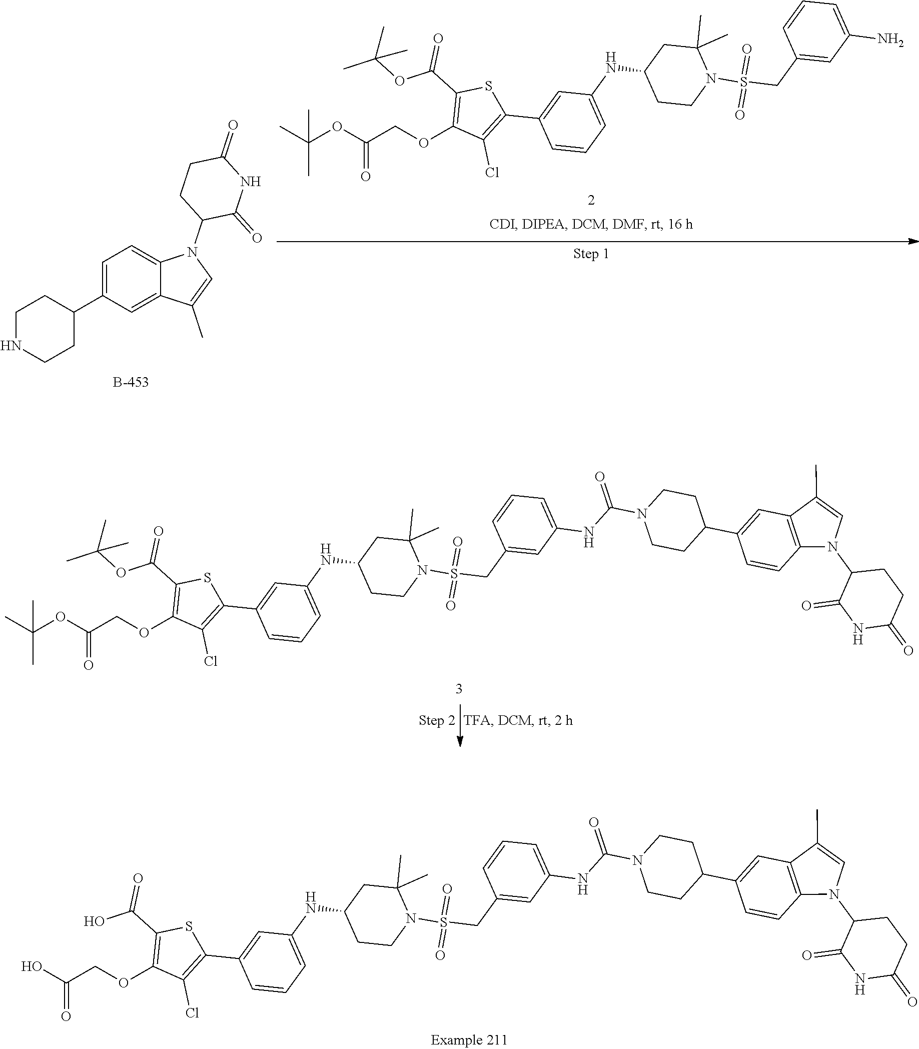

Step 1: tert-butyl 3-(2-tert-butoxy-2-oxo-ethoxy)-4-chloro-5-[3-[[(4S)-1-[[3-[[4-[1-(2,6-dioxo-3-piperidyl)-3-methyl-indol-5-yl]piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[[3-[4-[1-(2,6-dioxo-3-piperidyl)-3-methyl-indol-5-yl]piperidine-1-carbonyl]amino]phenyl]methylsulfonyl]-2,2-dimethyl-4-piperidyl]amino]phenyl]thiophene-2-carboxylate (3, 68 mg, 32.36 μmol, 59% yield, 51% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2, 40 mg, 54.97 μmol) and 3-[3-methyl-5-(4-piperidyl) indol-1-yl]piperidine-2,6-dione (B-453, 34.02 mg, 54.97 μmol, TFA salt) in a similar fashion to Compound B-242. The residue was treated with 1.5N HCl and the solid was filtered, washed with water and dried under vacuum to afford product. The material was taken to next step without purification.

LCMS (ES+): m/z 1071.2 [M+H]⁺

### Step 2: 3-(carboxymethoxy)-4-chloro-5-[3-[[(4S)-1-[3-[4-[1-(2,6-dioxo-3-piperidyl)-3-methyl-indol-5-yl]piperidine-1-carbonyl]amino]phenyl]methylsulfonyl]-2,2-dimethyl-4-piperidyl]amino]phenyl]thiophene-2-carboxylic acid (211)

3-(carboxymethoxy)-4-chloro-5-[3-[[(4S)-1-[[3-[[4-[1-(2,6-dioxo-3-piperidyl)-3-methyl-indol-5-yl]piperidine-1-carbonyl]amino]phenyl]methylsulfonyl]-2,2-dimethyl-4-piperidyl]amino]phenyl]thiophene-2-carboxylic acid (211, 15 mg, 15.48 μmol, 48% yield) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-3-methyl-indol-5-yl]piperidine-1-carbonyl]amino]phenyl]methylsulfonyl]-2,2-dimethyl-4-piperidyl]amino]phenyl]thiophene-2-carboxylate (3, 68 mg, 32.36 μmol) in a similar fashion to Compound A-26. The residue was purified by reverse phase prep HPLC [Purification method: X-Bridge C18 (150×30) mm, 5 micron; Mobile phase A: 10 Mm ammonium acetate in water; Mobile phase B: Acetonitrile].

LCMS (ES+): m/z 960.7 [M+H]⁺

¹HNMR (400 MHz, DMSO-d6): δ 11.02 (s, 1H), 8.63 (s, 1H), 7.58 (s, 1H), 7.48 (d, J=8.40 Hz, 1H), 7.34-7.23 (m, 3H), 7.19-7.15 (m, 1H), 7.09-6.98 (m, 4H), 6.83 (s, 1H), 6.77 (d, J=7.60 Hz, 1H), 6.65 (d, J=7.60 Hz, 1H), 5.62 (d, J=7.60 Hz, 1H), 5.53-5.48 (m, 1H), 4.62 (s, 2H), 4.38-4.24 (m, 4H), 2.95-2.79 (m, 6H), 2.68-2.63 (m, 2H), 2.23 (s, 3H), 2.11-2.08 (m, 2H), 1.49-1.40 (m, 7H), 1.31-1.17 (m, 3H).

### Example 212

### 3-(carboxymethoxy)-4-chloro-5-[3-[[(4S)-1-[3-[3-[[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (212)

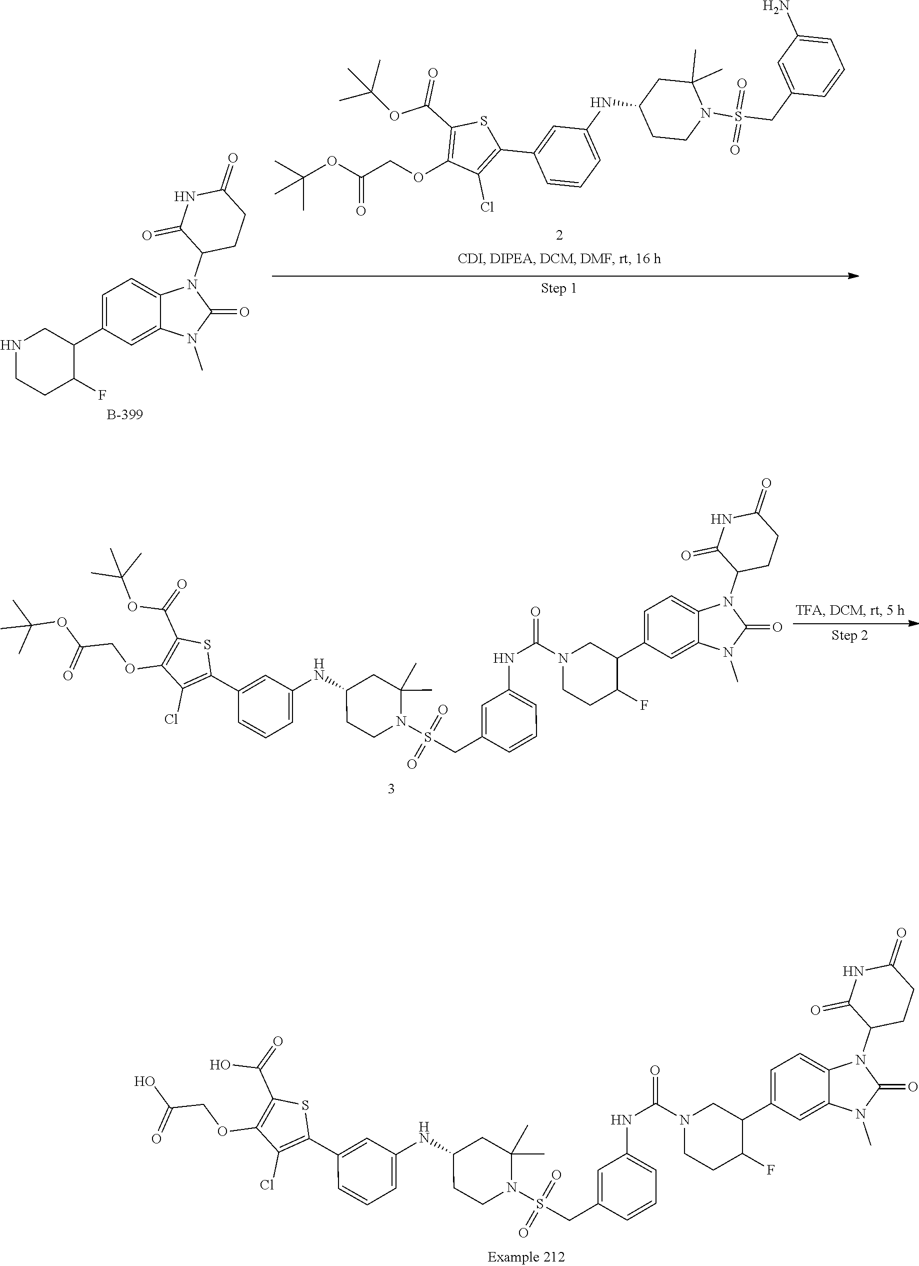

### Step 1: tert-butyl 3-(2-tert-butoxy-2-oxo-ethoxy)-4-chloro-5-[3-[[(4S)-1-[[3-[3-[[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylate (3)

tert-butyl 3-(2-tert-butoxy-2-oxo-ethoxy)-4-chloro-5-[3-[[(4S)-1-[[3-[[3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylate (3, 230 mg, 120.43 μmol, 58% yield, 58% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (2, 150 mg, 206.15 μmol) and 3-[5-(4-fluoro-3-piperidyl)-3-methyl-2-oxo-benzimidazol-1-yl]piperidine-2,6-dione (B-399, 100.83 mg, 206.15 μmol, TFA salt) in a similar fashion to Compound B-242. After completion of the reaction, excess solvent was removed from the reaction mixture and the residue was suspended in aqueous 1.5 N HCl (30 mL). The solid was filtered and washed with water and dried under vacuum. LCMS (ES+): m/z 1107.2 [M+H]⁺

### Step 2: 3-(carboxymethoxy)-4-chloro-5-[3-[[(4S)-1-[[3-[[3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (212)

3-(carboxymethoxy)-4-chloro-5-[3-[[(4S)-1-[[3-[[3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (212, 70 mg, 63.08 μmol, 53% yield, TFA salt) was synthesized from tert-butyl 3-(2-tert-butoxy-2-oxo-ethoxy)-4-chloro-5-[3-[[(4S)-1-[[3-[[3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylate (3, 230 mg, 118.46 μmol) in DCM (2.5 mL) in a similar fashion to Compound A-26. The residue was purified by reverse phase prep HPLC [Purification method: Column: XSelect, C18 (150×19) mm, 5 micron; Mobile phase A: 0.1% TFA in water; Mobile phase B: MeCN].

LCMS (ES+): m/z 994.0 [M+H]⁺

¹HNMR (400 MHz, DMSO-d6): δ 11.11 (s, 1H), 8.74 (s, 1H), 7.56 (s, 1H), 7.45 (d, J=8.40 Hz, 1H), 7.26-7.17 (m, 3H), 7.11-7.04 (m, 2H), 7.00 (d, J=7.20 Hz, 1H), 6.88 (s, 1H), 6.82 (d, J=7.60 Hz, 1H), 6.68 (d, J=8.00 Hz, 1H), 5.40-5.35 (m, 1H), 5.11-5.01 (m, 1H), 4.92 (s, 2H), 4.38-4.18 (m, 3H), 3.53-3.45 (m, 2H), 3.35 (s, 3H), 3.16-3.00 (m, 3H), 2.91-2.85 (m, 2H), 2.75-2.65 (m, 2H), 2.21-2.16 (m, 1H), 2.03-2.00 (m, 1H), 1.87-1.67 (m, 3H), 1.48-1.39 (m, 7H), 1.13-1.09 (m, 1H).

### Example 213

### 3-(carboxymethoxy)-4-chloro-5-[3-[[(4S)-1-[[3-[[(3R,4R)-3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (213)

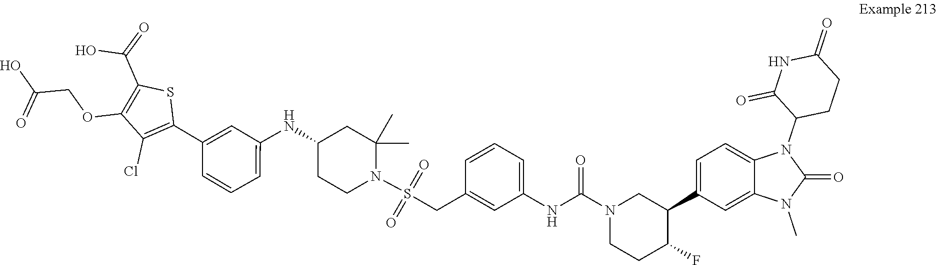 [SMILES: Cn1c(=O)n(C2CCC(=O)NC2=O)c2ccc([C@@H]3CN(C(=O)Nc4cccc(CS(=O)(=O)N5CC[C@H](Nc6cccc(-c7sc(C(=O)O)c(OCC(=O)O)c7Cl)c6)CC5(C)C)c4)CC[C@H]3F)cc21]

3-(carboxymethoxy)-4-chloro-5-[3-[[(4S)-1-[[3-[[(3R,4R)-3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (213, 110 mg, 98.58 μmol, 59% yield, TFA salt) was synthesized following the same procedure as (212).

LCMS (ES+): m/z 994.0 [M+H]⁺

¹HNMR (400 MHz, DMSO-d6): δ 11.11 (s, 1H), 8.74 (s, 1H), 7.56 (s, 1H), 7.45 (d, J=8.80 Hz, 1H), 7.26-7.17 (m, 3H), 7.11-6.99 (m, 3H), 6.88 (s, 1H), 6.82 (d, J=8.00 Hz, 1H), 6.69 (d, J=8.40 Hz, 1H), 5.40-5.35 (m, 1H), 5.11-5.01 (m, 1H), 4.93 (s, 2H), 4.38-4.18 (m, 3H), 3.54-3.45 (m, 2H), 3.35 (s, 3H), 3.13-3.03 (m, 2H), 2.91-2.87 (m, 2H), 2.68-2.61 (m, 2H), 2.25-2.15 (m, 1H), 2.08-1.99 (m, 1H), 1.88-1.61 (m, 3H), 1.48-1.41 (m, 7H), 1.15-1.05 (m, 1H).

### Example 214

### 3-(carboxymethoxy)-4-chloro-5-[3-[[(4S)-1-[[3-[[(3S,4S)-3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (214)

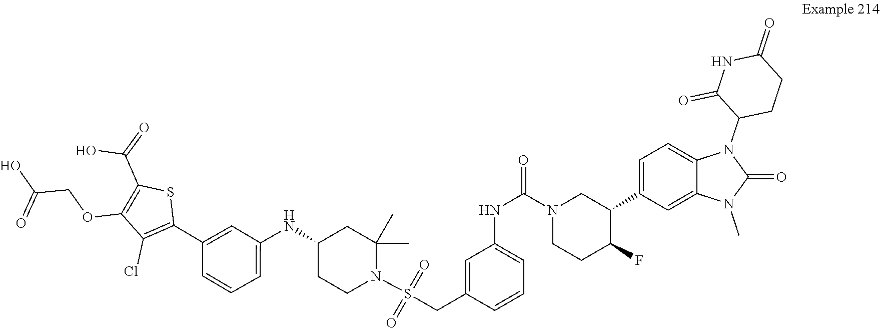 [SMILES: Cn1c(=O)n(C2CCC(=O)NC2=O)c2ccc([C@H]3CN(C(=O)Nc4cccc(CS(=O)(=O)N5CC[C@H](Nc6cccc(-c7sc(C(=O)O)c(OCC(=O)O)c7Cl)c6)CC5(C)C)c4)CC[C@@H]3F)cc21]

3-(carboxymethoxy)-4-chloro-5-[3-[[(4S)-1-[3-[(3S,4S)-3-[1-(2,6-dioxo-3-piperidyl)-3-methyl-2-oxo-benzimidazol-5-yl]-4-fluoro-piperidine-1-carbonyl]amino]phenyl]methylsulfonyl]-2,2-dimethyl-4-piperidyl]amino]phenyl]thiophene-2-carboxylic acid (214, 101 mg, 87.34 μmol, 55% yield, TFA salt) was synthesized following the same procedure as (212).

LCMS (ES+): m/z 994.0 [M+H]⁺

¹HNMR (400 MHz, DMSO-d6): δ 11.11 (s, 1H), 8.74 (s, 1H), 7.56 (s, 1H), 7.45 (d, J=8.40 Hz, 1H), 7.26-7.16 (m, 3H), 7.11-6.99 (m, 3H), 6.88 (s, 1H), 6.82 (d, J=7.60 Hz, 1H), 6.68 (d, J=8.00 Hz, 1H), 5.40-5.35 (m, 1H), 5.11-5.01 (m, 1H), 4.93 (s, 2H), 4.38-4.18 (m, 3H), 4.12-3.44 (m, 2H), 3.35 (s, 3H), 3.12-3.04 (m, 3H), 2.91-2.87 (m, 2H), 2.68-2.65 (m, 2H), 2.25-2.15 (m, 1H), 2.08-1.99 (m, 1H), 1.88-1.61 (m, 3H), 1.49-1.39 (m, 7H), 1.15-1.05 (m, 1H).

### Example 215

### 3-(carboxymethoxy)-4-chloro-5-[3-[[(4S)-1-[[3-[3-[[1-(2,6-dioxo-3-piperidyl)-2-oxo-benzo[cd]indol-4-yl]propylcarbamoylamino]phenyl]methylsulfonyl]-2,2-dimethyl-4-piperidyl]amino]phenyl]thiophene-2-carboxylic acid (215)

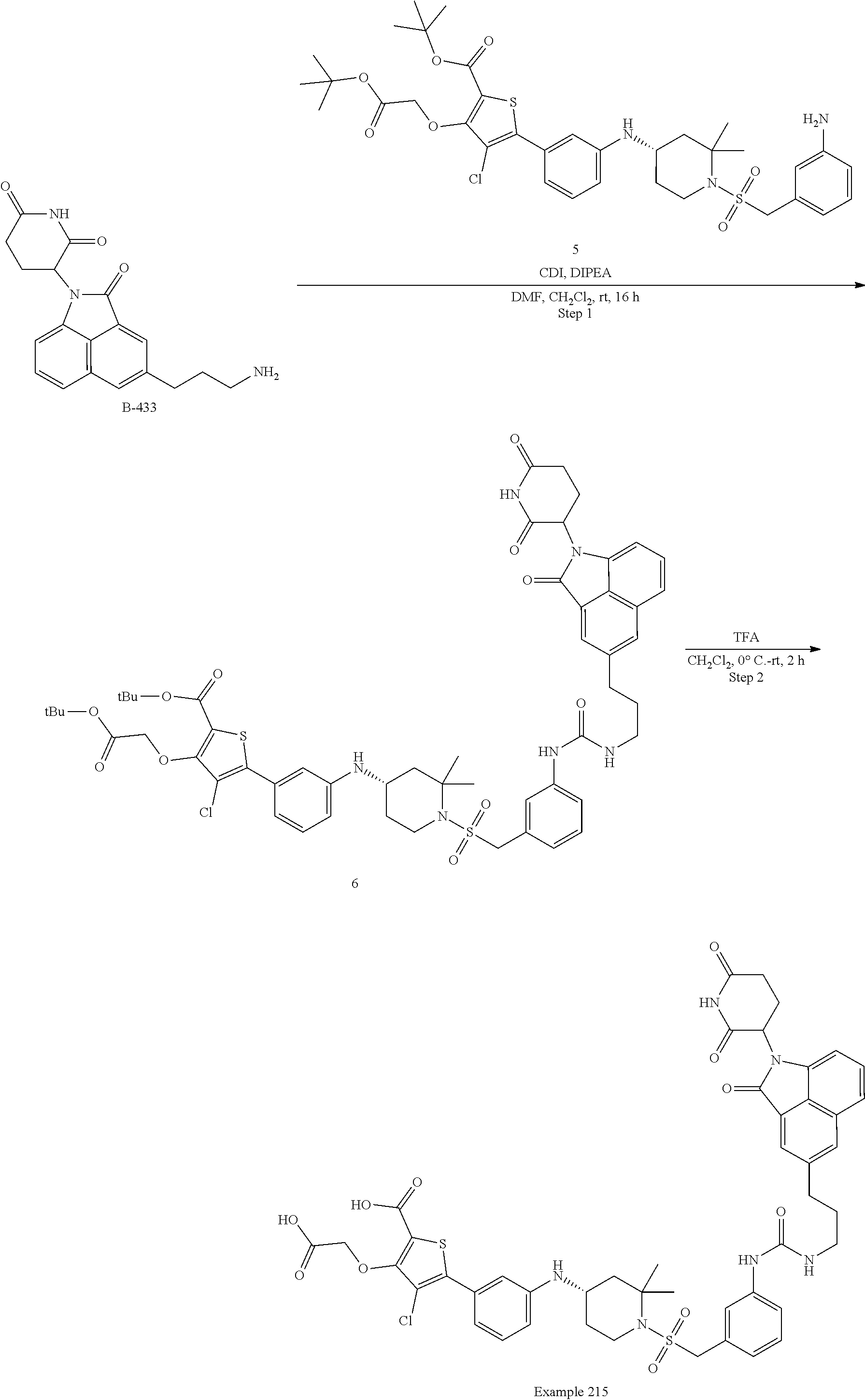 [SMILES: NCCCc1cc2c3c(cccc3c1)N(C1CCC(=O)NC1=O)C2=O ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)NCCCc5cc6c7c(cccc7c5)N(C5CCC(=O)NC5=O)C6=O)c4)C(C)(C)C3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)NCCCc2cc3c4c(cccc4c2)N(C2CCC(=O)NC2=O)C3=O)c1]

### Step 1: tert-butyl 3-(2-tert-butoxy-2-oxo-ethoxy)-4-chloro-5-[3-[[(4S)-1-[3-[3-[[1-(2,6-dioxo-3-piperidyl)-2-oxo-benzo[cd]indol-4-yl]propylcarbamoylamino]phenyl]methylsulfonyl]-2,2-dimethyl-4-piperidyl]amino]phenyl]thiophene-2-carboxylate (6)

tert-butyl 3-(2-tert-butoxy-2-oxo-ethoxy)-4-chloro-5-[3-[[(4S)-1-[[3-[3-[[1-(2,6-dioxo-3-piperidyl)-2-oxo-benzo[cd]indol-4-yl]propylcarbamoylamino]phenyl]methylsulfonyl]-2,2-dimethyl-4-piperidyl]amino]phenyl]thiophene-2-carboxylate (6, 300 mg, 209.25 μmol, 43% yield, 76% purity) is synthesized from tert-butyl 5-[3-[[(4S)-1-[(3-aminophenyl)methyl sulfonyl]-2,2-dimethyl-4-piperidyl]amino]phenyl]-3-(2-tert-butoxy-2-oxo-ethoxy)-4-chloro-thiophene-2-carboxylate (5, 348.83 mg, 481.84 μmol) and 3-[4-(3-aminopropyl)-2-oxo-benzo[cd]indol-1-yl]piperidine-2,6-dione (B-433, 250 mg, 481.84 μmol, TFA salt) in a similar fashion to Compound B-242. The solvent was evaporated under reduced pressure and the residue was diluted with ice cold water to precipitate a solid that was filtered and dried under vacuum. LCMS (ES+): m/z 1085.2 [M+H]⁺

### Step 2: 3-(carboxymethoxy)-4-chloro-5-[3-[[(4S)-1-[[3-[3-[1-(2,6-dioxo-3-piperidyl)-2-oxo-benzo[cd]indol-4-yl]propylcarbamoylamino]phenyl]methylsulfonyl]-2,2-dimethyl-4-piperidyl]amino]phenyl]thiophene-2-carboxylic acid (215)

3-(carboxymethoxy)-4-chloro-5-[3-[[(4S)-1-[3-[3-[[1-(2,6-dioxo-3-piperidyl)-2-oxo-benzo[cd]indol-4-yl]propylcarbamoylamino]phenyl]methylsulfonyl]-2,2-dimethyl-4-piperidyl]amino]phenyl]thiophene-2-carboxylic acid (215, 130 mg, 111.35 μmol, 53% yield, TFA salt) was synthesized from tert-butyl 3-(2-tert-butoxy-2-oxo-ethoxy)-4-chloro-5-[3-[[(4S)-1-[[3-[3-[[1-(2,6-dioxo-3-piperidyl)-2-oxo-benzo[cd]indol-4-yl]propylcarbamoylamino]phenyl]methylsulfonyl]-2,2-dimethyl-4-piperidyl]amino]phenyl]thiophene-2-carboxylate (6, 300 mg, 209.25 μmol) in a similar fashion to Compound A-26. The residue was purified by reverse-phase preparative HPLC [Column: X Select C18 (250×19) mm, 5 μm; Mobile Phase A: 0.1% TFA in water and Mobile Phase B: Acetonitrile].

¹H-NMR (400 MHz, DMSO-d6): δ 11.12 (s, 1H), 8.54 (s, 1H), 8.03 (d, J=13.20 Hz, 1H), 7.60 (d, J=8.80 Hz, 1H), 7.50 (t, J=8.00 Hz, 1H), 7.46 (s, 1H), 7.41 (d, J=8.40 Hz, 1H), 7.25-7.17 (m, 2H), 7.10 (d, J=7.60 Hz, 1H), 6.94 (d, J=7.60 Hz, 1H), 6.88 (s, 1H), 6.81 (d, J=7.60 Hz, 1H), 6.69 (d, J=8.00 Hz, 1H), 6.22-6.18 (m, 1H), 5.44 (dd, J=5.20, 12.80 Hz, 1H), 4.92 (s, 2H), 4.38-4.23 (m, 2H), 3.47-3.43 (m, 2H), 3.17-3.12 (m, 3H), 2.96-2.92 (m, 3H), 2.68-2.64 (m, 2H), 2.20-2.08 (m, 1H), 1.88 (s, 4H), 1.46-1.39 (m, 7H), 1.20-1.08 (m, 1H). LCMS (ES+): m/z 972.0 [M+H]⁺

### Example 216

### 3-(carboxymethoxy)-4-chloro-5-[3-[[(4S)-1-[3-[[1-[1-(2,6-dioxo-3-piperidyl)-2-oxo-benzo[cd]indol-5-yl]piperidine-4-carbonyl]amino]phenyl]methylsulfonyl]-2,2-dimethyl-4-piperidyl]amino]phenyl]thiophene-2-carboxylic acid (216)

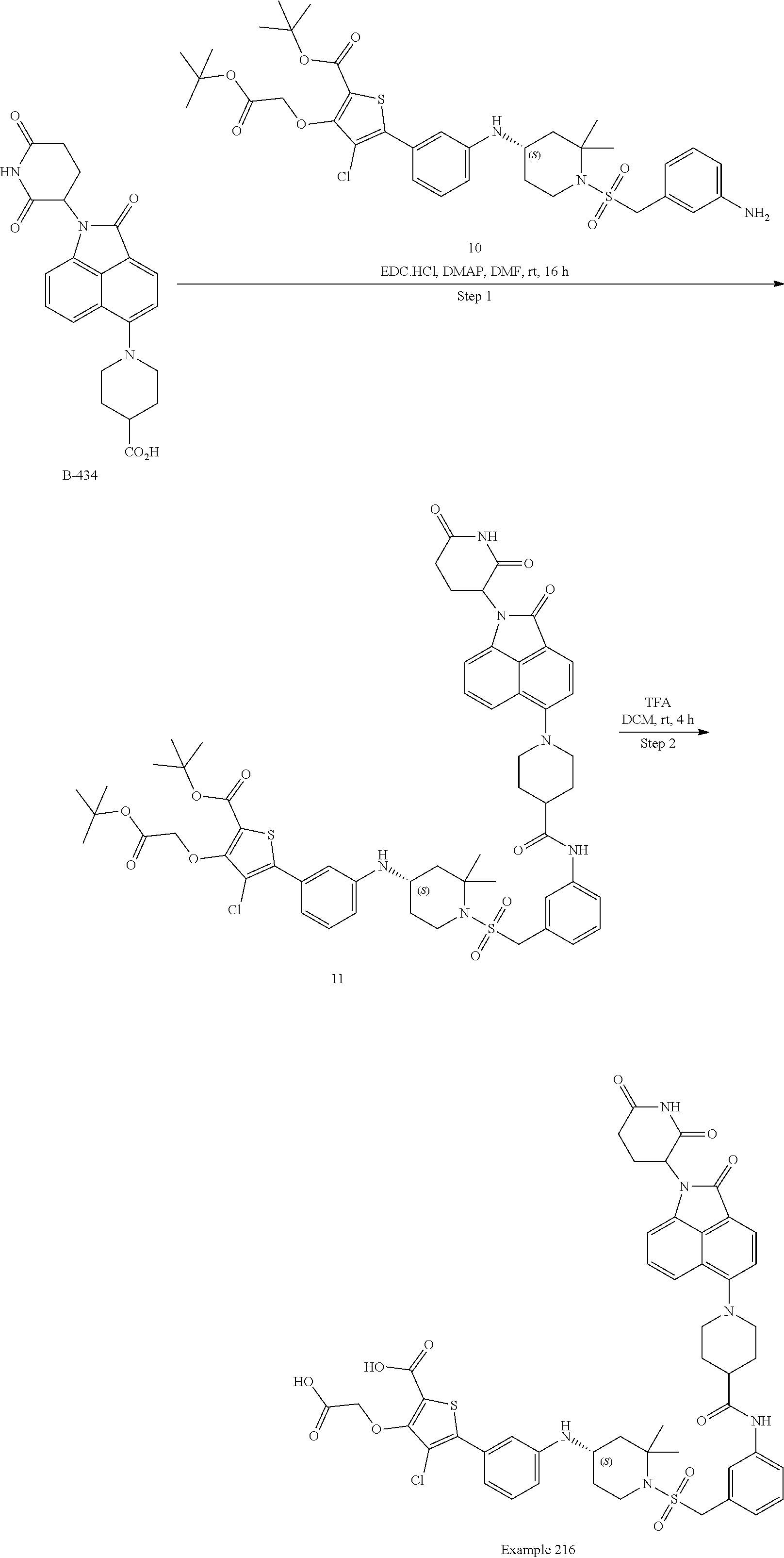

### Step 7: tert-butyl 3-(2-tert-butoxy-2-oxo-ethoxy)-4-chloro-5-[3-[[(4S)-1-[[3-[[1-[1-(2,6-dioxo-3-piperidyl)-2-oxo-benzo[cd]indol-5-yl]piperidine-4-carbonyl]amino]phenyl]methylsulfonyl]-2,2-dimethyl-4-piperidyl]amino]phenyl]thiophene-2-carboxylate (11)

tert-butyl 3-(2-tert-butoxy-2-oxo-ethoxy)-4-chloro-5-[3-[[(4S)-1-[3-[[1-[1-(2,6-dioxo-3-piperidyl)-2-oxo-benzo[cd]indol-5-yl]piperidine-4-carbonyl]amino]phenyl]methylsulfonyl]-2,2-dimethyl-4-piperidyl]amino]phenyl]thiophene-2-carboxylate (11, 0.24 g, 83.24 μmol, 48% yield, 38% purity) was synthesized from 1-[1-(2,6-dioxo-3-piperidyl)-2-oxo-benzo[cd]indol-5-yl]piperidine-4-carboxylic acid (B-434, 0.08 g, 173.85 μmol) and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (10, 151.09 mg, 208.61 μmol in a similar fashion to Example 166. The residue was suspended in cold water (20 mL) and the solid was filtered and dried. The material was used in the next step without further purification. LCMS (ES+): m/z 1109.3 [M+H]⁺

### Step 8: 3-(carboxymethoxy)-4-chloro-S-[3-[[(4S)-1-[[3-[[1-[1-(2,6-dioxo-3-piperidyl)-2-oxo-benzo[cd]indol-5-yl]piperidine-4-carbonyl]amino]phenyl]methylsulfonyl]-2,2-dimethyl-4-piperidyl]amino]phenyl]thiophene-2-carboxylic acid (216)

3-(carboxymethoxy)-4-chloro-5-[3-[[(4S)-1-[3-[[1-[1-(2,6-dioxo-3-piperidyl)-2-oxo-benzo[cd]indol-5-yl]piperidine-4-carbonyl]amino]phenyl]methylsulfonyl]-2,2-dimethyl-4-piperidyl]amino]phenyl]thiophene-2-carboxylic acid (216, 50 mg, 42.92 μmol, 52% yield, TFA salt) was synthesized from tert-butyl 3-(2-tert-butoxy-2-oxo-ethoxy)-4-chloro-5-[3-[[(4S)-1-[3-[[1-[1-(2,6-dioxo-3-piperidyl)-2-oxo-benzo[cd]indol-5-yl]piperidine-4-carbonyl]amino]phenyl]methylsulfonyl]-2,2-dimethyl-4-piperidyl]amino]phenyl]thiophene-2-carboxylate (11, 0.24 g, 83.24 μmol) in a similar fashion to Compound A-26. The residue was purified by reverse phase prep HPLC [Purification method: Column: XSelect, C18 (150×30) mm, 5 micron; Mobile phase A: 0.1% TFA in water and Mobile phase B: MeCN. LCMS (ES+): m/z 997.0 [M+H]⁺

¹HNMR (400 MHz, DMSO-d6): δ 11.11 (s, 1H), 10.09 (s, 1H), 7.96 (d, J=7.60 Hz, 1H), 7.78 (s, 1H), 7.64-7.60 (m, 2H), 7.46-7.42 (m, 1H), 7.35-7.31 (m, 1H), 7.20-7.16 (m, 2H), 7.11-7.07 (m, 2H), 6.88 (s, 1H), 6.80 (d, J=7.60 Hz, 1H), 6.71 (d, J=8.80 Hz, 1H), 5.43-5.39 (m, 1H), 4.89 (s, 2H), 4.37 (dd, J=14.00, 44.60 Hz, 1H), 3.82-3.79 (m, 1H), 3.45-3.41 (m, 1H), 3.13-2.95 (m, 4H), 2.70-2.55 (m, 3H), 2.08-2.02 (m, 6H), 1.89-1.79 (m, 3H), 1.49 (s, 3H), 1.40 (s, 3H), 1.14-1.12 (m, 1H).

### Example 217

### 3-(carboxymethoxy)-4-chloro-5-[3-[[(4S)-1-[3-[[1-[1-(2,6-dioxo-3-piperidyl)-2-oxo-benzo[cd]indol-6-yl]piperidine-4-carbonyl]amino]phenyl]methylsulfonyl]-2,2-dimethyl-4-piperidyl]amino]phenyl]thiophene-2-carboxylic acid (217)

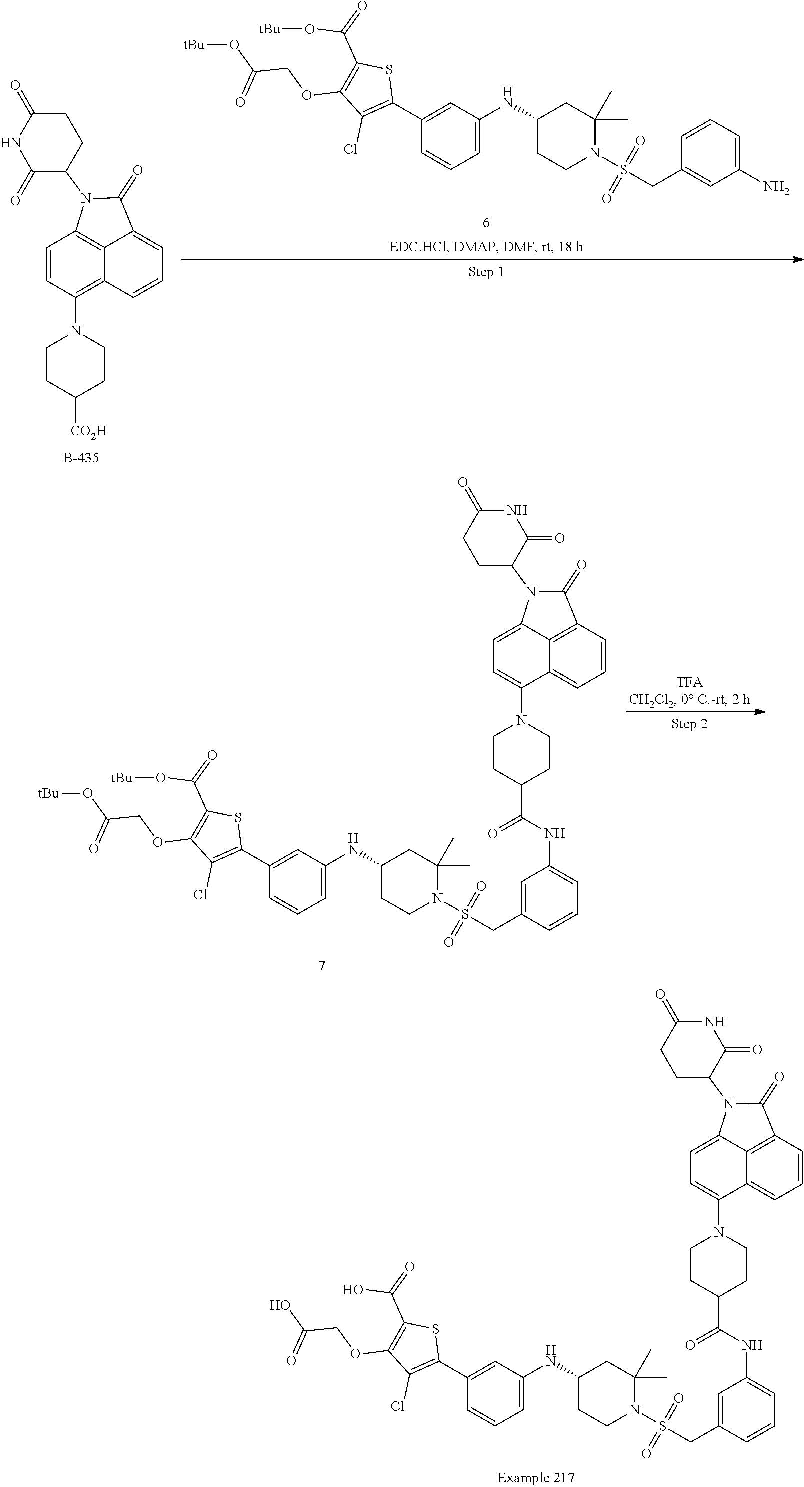 [SMILES: O=C1CCC(N2C(=O)c3cccc4c(N5CCC(C(=O)O)CC5)ccc2c34)C(=O)N1 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)C5CCN(c6ccc7c8c(cccc68)C(=O)N7C6CCC(=O)NC6=O)CC5)c4)C(C)(C)C3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)C2CCN(c3ccc4c5c(cccc35)C(=O)N4C3CCC(=O)NC3=O)CC2)c1]

### Step 4: tert-butyl 3-(2-tert-butoxy-2-oxo-ethoxy)-4-chloro-5-[3-[[(4S)-1-[[3-[[1-[1-(2,6-dioxo-3-piperidyl)-2-oxo-benzo[cd]indol-6-yl]piperidine-4-carbonyl]amino]phenyl]methylsulfonyl]-2,2-dimethyl-4-piperidyl]amino]phenyl]thiophene-2-carboxylate (7)

tert-butyl 3-(2-tert-butoxy-2-oxo-ethoxy)-4-chloro-5-[3-[[(4S)-1-[[3-[[1-[1-(2,6-dioxo-3-piperidyl)-2-oxo-benzo[cd]indol-6-yl]piperidine-4-carbonyl]amino]phenyl]methylsulfonyl]-2,2-dimethyl-4-piperidyl]amino]phenyl]thiophene-2-carboxylate (7, 240 mg, 88.67 μmol, 41% yield, 41% purity) was synthesized from 1-[1-(2,6-dioxo-3-piperidyl)-2-oxo-benzo[cd]indol-6-yl]piperidine-4-carboxylic acid (B-435, 125 mg, 214.77 μmol and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (6, 156.27 mg, 214.77 μmol) in a similar fashion to Example 166. The solvent was then evaporated under reduced pressure and diluted with ice-cold water. The precipitated solid was filtered and dried.

LCMS (ES+): m/z 1109.4 [M+H]⁺

### Step 5: 3-(carboxymethoxy)-4-chloro-5-[3-[[(4S)-1-[3-[[1-[1-(2,6-dioxo-3-piperidyl)-2-oxo-benzo[cd]indol-6-yl]piperidine-4-carbonyl]amino]phenyl]methylsulfonyl]-2,2-dimethyl-4-piperidyl]amino]phenyl]thiophene-2-carboxylic acid (217)

3-(carboxymethoxy)-4-chloro-5-[3-[[(4S)-1-[3-[[1-[1-(2,6-dioxo-3-piperidyl)-2-oxo-benzo[cd]indol-6-yl]piperidine-4-carbonyl]amino]phenyl]methylsulfonyl]-2,2-dimethyl-4-piperidyl]amino]phenyl]thiophene-2-carboxylic acid (217, 25 mg, 22.23 μmol, 25% yield, TFA salt) was synthesized from tert-butyl 3-(2-tert-butoxy-2-oxo-ethoxy)-4-chloro-5-[3-[[(4S)-1-[[3-[1-[1-(2,6-dioxo-3-piperidyl)-2-oxo-benzo[cd]indol-6-yl]piperidine-4-carbonyl]amino]phenyl]methylsulfonyl]-2,2-dimethyl-4-piperidyl]amino]phenyl]thiophene-2-carboxylate (240 mg, 88.67 μmol) in a similar fashion to Compound A-26. The residue was purified by reverse phase prep HPLC [Purification method: Column: Sunfire C18 (19×150) mm, 5 micron; Mobile Phase A: 0.1% TFA in water; Mobile Phase B: MeCN].

LCMS (ES+): m/z 996.7 [M+H]⁺

¹HNMR (400 MHz, DMSO-d6): δ 13.10 (bs, 1H), 11.13 (s, 1H), 10.07 (s, 1H), 8.26 (d, J=8.40 Hz, 1H), 8.09 (d, J=7.20 Hz, 1H), 7.84-7.80 (m, 1H), 7.77 (s, 1H), 7.64 (d, J=8.40 Hz, 1H), 7.33 (t, J=8.00 Hz, 1H), 7.19 (t, J=7.60 Hz, 1H), 7.10 (d, J=8.00 Hz, 1H), 7.03 (d, J=7.60 Hz, 1H), 6.98 (d, J=8.00 Hz, 1H), 6.89 (d, J=Hz, 1H), 6.81 (d, J=8.00 Hz, 1H), 6.72 (d, J=9.60 Hz, 1H), 5.45-5.41 (m, 1H), 4.88 (s, 2H), 4.37 (dd, J=14.00, 44.40 Hz, 2H), 3.55-3.45 (m, 2H), 3.17-3.01 (m, 1H), 2.99-2.92 (m, 1H), 2.83-2.52 (m, 4H), 2.08-2.00 (m, 6H), 1.90-1.79 (m, 2H), 1.49-1.41 (m, 7H).

### Example 218

### 3-(carboxymethoxy)-4-chloro-5-[3-[[(4S)-1-[[3-[[4-[9-(2,6-dioxo-3-piperidyl)pyrido[2,3-b]indol-3-yl]piperidine-1-carbonyl]amino]phenyl]methylsulfonyl]-2,2-dimethyl-4-piperidyl]amino]phenyl]thiophene-2-carboxylic acid (218)

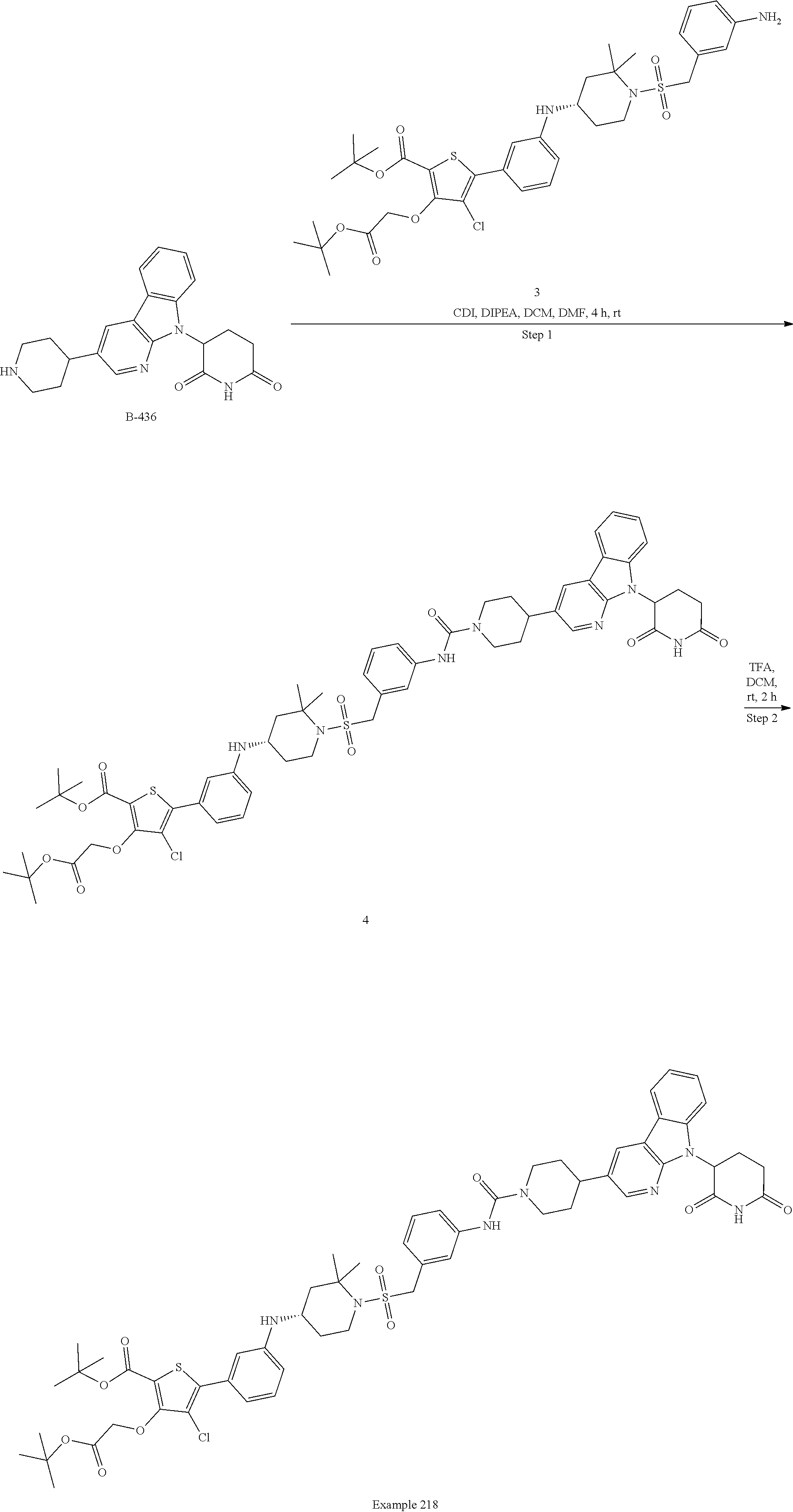 [SMILES: O=C1CCC(n2c3ccccc3c3cc(C4CCNCC4)cnc32)C(=O)N1 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)N5CCC(c6cnc7c(c6)c6ccccc6n7C6CCC(=O)NC6=O)CC5)c4)C(C)(C)C3)c2)c1Cl ; CC(C)(C)OC(=O)COc1c(C(=O)OC(C)(C)C)sc(-c2cccc(N[C@H]3CCN(S(=O)(=O)Cc4cccc(NC(=O)N5CCC(c6cnc7c(c6)c6ccccc6n7C6CCC(=O)NC6=O)CC5)c4)C(C)(C)C3)c2)c1Cl]

### Step 1: tert-butyl 3-(2-tert-butoxy-2-oxo-ethoxy)-4-chloro-5-[3-[[(4S)-1-[[3-[[4-[9-(2,6-dioxo-3-piperidyl)pyrido[2,3-b]indol-3-yl]piperidine-1-carbonyl]amino]phenyl]methylsulfonyl]-2,2-dimethyl-4-piperidyl]amino]phenyl]thiophene-2-carboxylate (4)

tert-butyl 3-(2-tert-butoxy-2-oxo-ethoxy)-4-chloro-5-[3-[[(4S)-1-[[3-[[4-[9-(2,6-dioxo-3-piperidyl)pyrido[2,3-b]indol-3-yl]piperidine-1-carbonyl]amino]phenyl]methylsulfonyl]-2,2-dimethyl-4-piperidyl]amino]phenyl]thiophene-2-carboxylate (120 mg, 44.37 μmol, 31% yield, 41% purity) was synthesized from 3-[3-(4-piperidyl)pyrido[2,3-b]indol-9-yl]piperidine-2,6-dione (B-436, 70 mg, 142.51 μmol, TFA) and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxy late (3, 102.66 mg, 142.51 μmol) in a similar fashion to Compound B-242. The residue was treated with water (2 mL) and the precipitate was filtered and dried. LCMS (ES+): m/z 1111.2 [M+H]⁺

### Step 2: 3-(carboxymethoxy)-4-chloro-5-[3-[[(4S)-1-[[3-[[4-[9-(2,6-dioxo-3-piperidyl)pyrido[2,3-b]indol-3-yl]piperidine-1-carbonyl]amino]phenyl]methylsulfonyl]-2,2-dimethyl-4-piperidyl]amino]phenyl]thiophene-2-carboxylic acid (218)

3-(carboxymethoxy)-4-chloro-5-[3-[[(4S)-1-[[3-[[4-[9-(2,6-dioxo-3-piperidyl)pyrido[2,3-b]indol-3-yl]piperidine-1-carbonyl]amino]phenyl]methylsulfonyl]-2,2-dimethyl-4-piperidyl]amino]phenyl]thiophene-2-carboxylic acid (218, 15 mg, 13.43 μmol, 24% yield, TFA salt) was synthesized from tert-butyl 3-(2-tert-butoxy-2-oxo-ethoxy)-4-chloro-5-[3-[[(4S)-1-[[3-[[4-[9-(2,6-dioxo-3-piperidyl)pyrido[2,3-b]indol-3-yl]piperidine-1-carbonyl]amino]phenyl]methylsulfonyl]-2,2-dimethyl-4-piperidyl]amino]phenyl]thiophene-2-carboxylate (80 mg, 56.59 μmol) in a similar fashion to Compound A-26. The residue was purified with reverse phase preparatory HPLC (Column: XSelect C18 (250×19) mm, 5 micron; Mobile phase A: 0.1% TFA in water; Mobile phase B: Acetonitrile]. LCMS (ES−): m/z 994.0 [M−H]^{- 1}HNMR (400 MHz, DMSO-d6): δ 13.13 (bs, 1H), 11.15 (s, 1H), 8.66 (s, 1H), 8.53 (s, 1H), 8.36 (s, 1H), 8.23 (d, J=7.60 Hz, 1H), 7.60 (s, 2H), 7.51-7.45 (m, 2H), 7.27-7.18 (m, 3H), 7.01-6.98 (m, 1H), 6.88 (s, 1H), 6.81 (d, J=7.60 Hz, 1H), 6.70 (d, J=8.00 Hz, 1H), 6.01 (s, 1H), 4.91 (s, 2H), 4.39-4.25 (m, 4H), 3.17-3.07 (m, 4H), 2.99-2.93 (m, 4H), 2.12-2.09 (m, 2H), 1.91-1.69 (m, 8H), 1.52-1.35 (m, 7H), 1.20-1.05 (m, 2H).

### Example 219

### 3-(carboxymethoxy)-4-chloro-5-(3-(((4S)-1-((3-(3-(2-(1-(1-(2,6-dioxopiperidin-3-yl)-2-oxo-1,2-dihydrobenzo[cd]indol-6-yl))piperidin-4-yl)ethyl)ureido)benzyl)sulfonyl)-2,2-dimethylpiperidin-4-yl)amino)phenyl)thiophene-2-carboxylic acid (219)

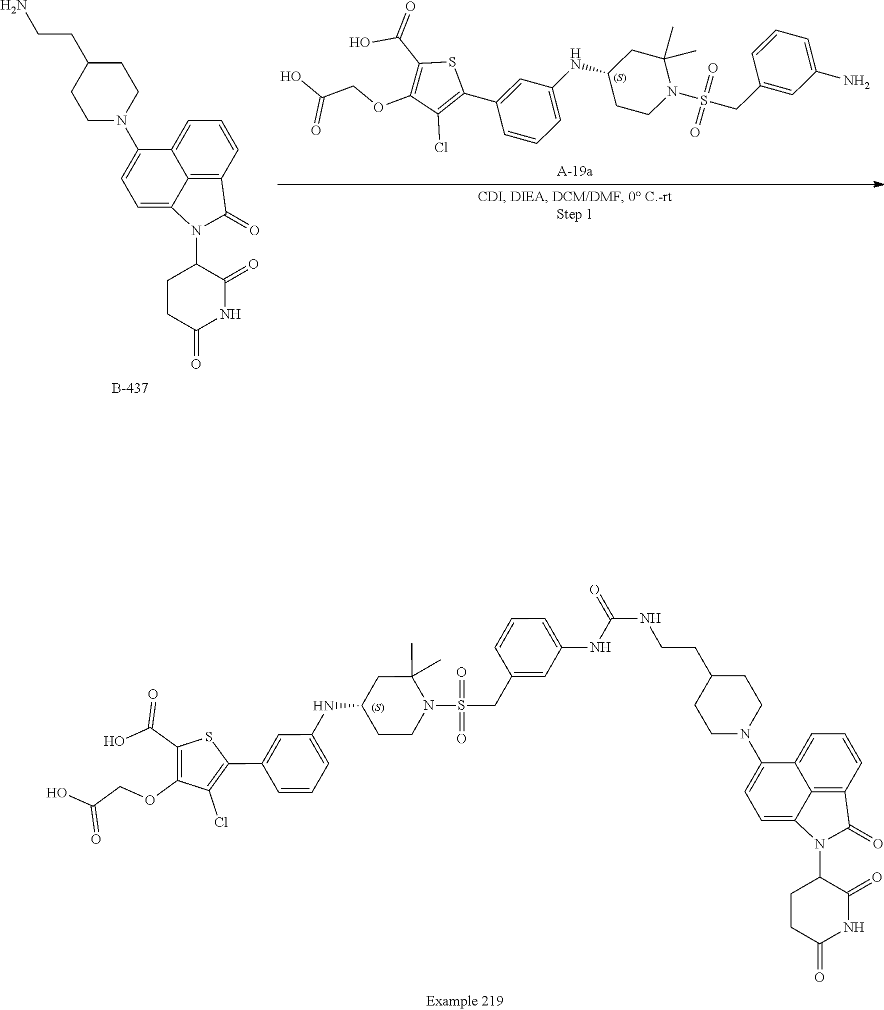

### Step 1: 3-(carboxymethoxy)-4-chloro-5-(3-(((4S)-1-((3-(3-(2-(1-(1-(2,6-dioxopiperidin-3-yl)-2-oxo-1,2-dihydrobenzo[cd]indol-6-yl)piperidin-4-yl)ethyl)ureido)benzyl)sulfonyl)-2,2-dimethylpiperidin-4-yl)amino)phenyl)thiophene-2-carboxylic acid (219)

3-(carboxymethoxy)-4-chloro-5-(3-(((4S)-1-((3-(3-(2-(1-(1-(2,6-dioxopiperidin-3-yl)-2-oxo-1,2-dihydrobenzo[cd]indol-6-yl)piperidin-4-ylethyl)ureido)benzyl)sulfonyl)-2,2-dimethylpiperidin-4-yl)amino)phenyl)thiophene-2-carboxylic acid (219, 64.12 mg, 54.98 μmol, 40% yield) was synthesized from(S)-5-(3-((1-((3-aminobenzyl)sulfonyl)-2,2-dimethylpiperidin-4-yl)amino)phenyl)-3-(carboxymethoxy)-4-chlorothiophene-2-carboxylic acid (A-19a, 82.38 mg, 135.46 μmol) and 3-(6-(4-(2-aminoethyl)piperidin-1-yl)-2-oxobenzo[cd]indol-[(2H)-yl)piperidine-2,6-dione (B-437, 60 mg, 135.46 μmol, HCl salt) in a similar fashion to Compound B-242. The residue was purified by prep-HPLC (TFA condition; flow: 25 mL/min; gradient: from 35-65% water (0.1% TFA)-ACN over 7 min; column; 3_Phenomenex Luna C18 75*30 mm*3 μm).

LCMS (ESI): m/z 1040.4 [M+H]⁺

¹H NMR (400 MHz, DMSO-d₆) δ=11.19-11.04 (m, 1H), 8.55-8.49 (m, 1H), 8.27-8.20 (m, 1H), 8.08 (d, J=7.0 Hz, 1H), 7.82 (t, J=7.7 Hz, 1H), 7.48 (s, 1H), 7.38 (br d, J=8.9 Hz, 1H), 7.21 (q, J=7.9 Hz, 2H), 7.04-6.91 (m, 3H), 6.88 (s, 1H), 6.82 (d, J=7.9 Hz, 1H), 6.74-6.68 (m, 1H), 6.17 (br d, J=2.4 Hz, 1H), 5.41 (dd, J=5.5, 12.7 Hz, 1H), 4.91 (s, 2H), 4.38-4.35 (m, 1H), 4.26 (br s, 1H), 3.59-3.48 (m, 1H), 3.48-3.38 (m, 3H), 3.31-3.05 (m, 3H), 3.02-2.69 (m, 2H), 2.68-2.56 (m, 2H), 2.07 (br dd, J=5.9, 11.0 Hz, 1H), 1.91-1.73 (m, 4H), 1.59-1.34 (m, 12H), 1.13 (br d, J=10.3 Hz, 1H).

### Example 220

### 3-(carboxymethoxy)-4-chloro-5-(3-(((4S)-1-((3-(4-(((1-(2,6-dioxopiperidin-3-yl)-2-oxo-1,2-dihydrobenzo[cd]indol-6-yl)methyl)amino)-4-oxobutanamido)benzyl)sulfonyl)-2,2-dimethylpiperidin-4-yl)amino)phenyl)thiophene-2-carboxylic acid (220)

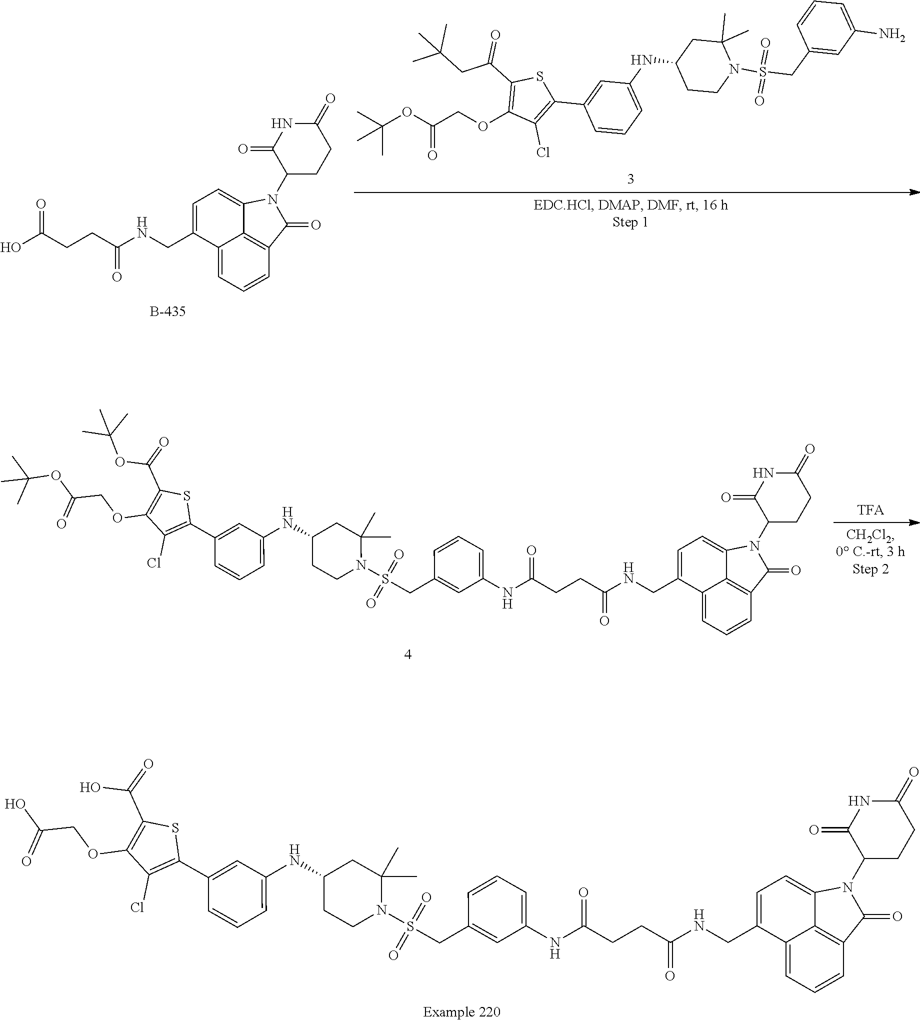

### Step 1: tert-butyl 3-(2-(tert-butoxy)-2-oxoethoxy)-4-chloro-5-(3-(((4S)-1-((3-(4-(((1-(2,6-dioxopiperidin-3-yl)-2-oxo-1,2-dihydrobenzo[cd]indol-6-yl)methyl)amino)-4-oxobutanamido)benzyl)sulfonyl)-2,2-dimethylpiperidin-4-yl)amino)phenyl)thiophene-2-carboxylate (4)

tert-butyl 3-(2-tert-butoxy-2-oxo-ethoxy)-4-chloro-5-[3-[[(4S)-1-[[3-[[4-[[1-(2,6-dioxo-3-piperidyl)-2-oxo-benzo[cd]indol-6-yl]methylamino]-4-oxo-butanoyl]amino]phenyl]methylsulfonyl]-2,2-dimethyl-4-piperidyl]amino]phenyl]thiophene-2-carboxylate (4, 200 mg, 98.05 μmol, 45% yield, 55% purity) was synthesized from 4-[[1-(2,6-dioxo-3-piperidyl)-2-oxo-benzo[cd]indol-6-yl]methylamino]-4-oxo-butanoic acid (B-438, 100 mg, 216.66 μmol) and tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (3, 189.18 mg, 260.00 μmol) in a similar fashion to Example 166. The reaction mixture was concentrated under reduced pressure and diluted with water (10 mL) to precipitate a solid that was filtered, and dried under vacuum.

LCMS (ES+): m/z 1111.0 [M+H]⁺

### Step 2: 3-(carboxymethoxy)-4-chloro-5-(3-(((4S)-1-((3-(4-(((1-(2,6-dioxopiperidin-3-yl)-2-oxo-1,2-dihydrobenzo[cd]indol-6-yl)methyl)amino)-4-oxobutanamido)benzyl)sulfonyl)-2,2-dimethylpiperidin-4-yl)amino)phenyl)thiophene-2-carboxylic acid (220)

3-(carboxymethoxy)-4-chloro-5-[3-[[(4S)-1-[3-[4-[[1-(2,6-dioxo-3-piperidyl)-2-oxo-benzo[cd]indol-6-yl]methylamino]-4-oxo-butanoyl]amino]phenyl]methylsulfonyl]-2,2-dimethyl-4-piperidyl]amino]phenyl]thiophene-2-carboxylic acid (220, 29 mg, 27.09 μmol, 28% yield, formic acid salt) was synthesized from tert-butyl 3-(2-tert-butoxy-2-oxo-ethoxy)-4-chloro-5-[3-[[(4S)-1-[3-[4-[1-(2,6-dioxo-3-piperidyl)-2-oxo-benzo[cd]indol-6-yl]methylamino]-4-oxo-butanoyl]amino]phenyl]methylsulfonyl]-2,2-dimethyl-4-piperidyl]amino]phenyl]thiophene-2-carboxylate (4, 200 mg, 98.05 μmol) in a similar fashion to Compound A-26. The residue was purified by reverse-phase preparative HPLC [Column: X-BRIDGE C18 (150×10) mm, 5 μm; Mobile phase A: 0.1% FA in water and Mobile Phase B: MeCN].

¹H-NMR (400 MHz, DMSO-d6): δ 11.15 (s, 1H), 10.05 (s, 1H), 8.49 (t, J=6.40 Hz, 1H), 8.35 (d, J=8.00 Hz, 1H), 8.09 (d, J=6.80 Hz, 1H), 7.82 (t, J=7.20 Hz, 1H), 7.70 (s, 1H), 7.58-7.57 (m, 1H), 7.43-7.41 (m, 1H), 7.30 (t, J=7.60 Hz, 1H), 7.21-7.16 (m, 1H), 7.08-7.02 (m, 2H), 6.87 (s, 1H), 6.79 (d, J=8.00 Hz, 1H), 6.68 (d, J=7.60 Hz, 1H), 5.80-5.70 (m, 1H), 5.44 (dd, J=5.20, 12.80 Hz, 1H), 4.84 (s, 2H), 4.70 (d, J=5.60 Hz, 2H), 4.42-4.26 (m, 2H), 3.34-2.92 (m, 2H), 2.77-2.63 (m, 4H), 2.18-2.01 (m, 1H), 1.83-1.76 (m, 2H), 1.46-1.39 (m, 9H), 1.24-1.07 (m, 1H).

[2 protons are merged in solvent peak] LCMS (ES+): m/z 999.2 [M+H]⁺.

### Example 221

### 3-(carboxymethoxy)-4-chloro-5-[3-[[(4S)-1-[[3-[[4-[[1-(2,6-dioxo-3-piperidyl)-3-methyl-2-oxo-benzimidazol-S-yl]methyl]piperazine-1-carbonyl]amino]phenyl]methylsulfonyl]-2,2-dimethyl-4-piperidyl]amino]phenyl]thiophene-2-carboxylic acid (221)

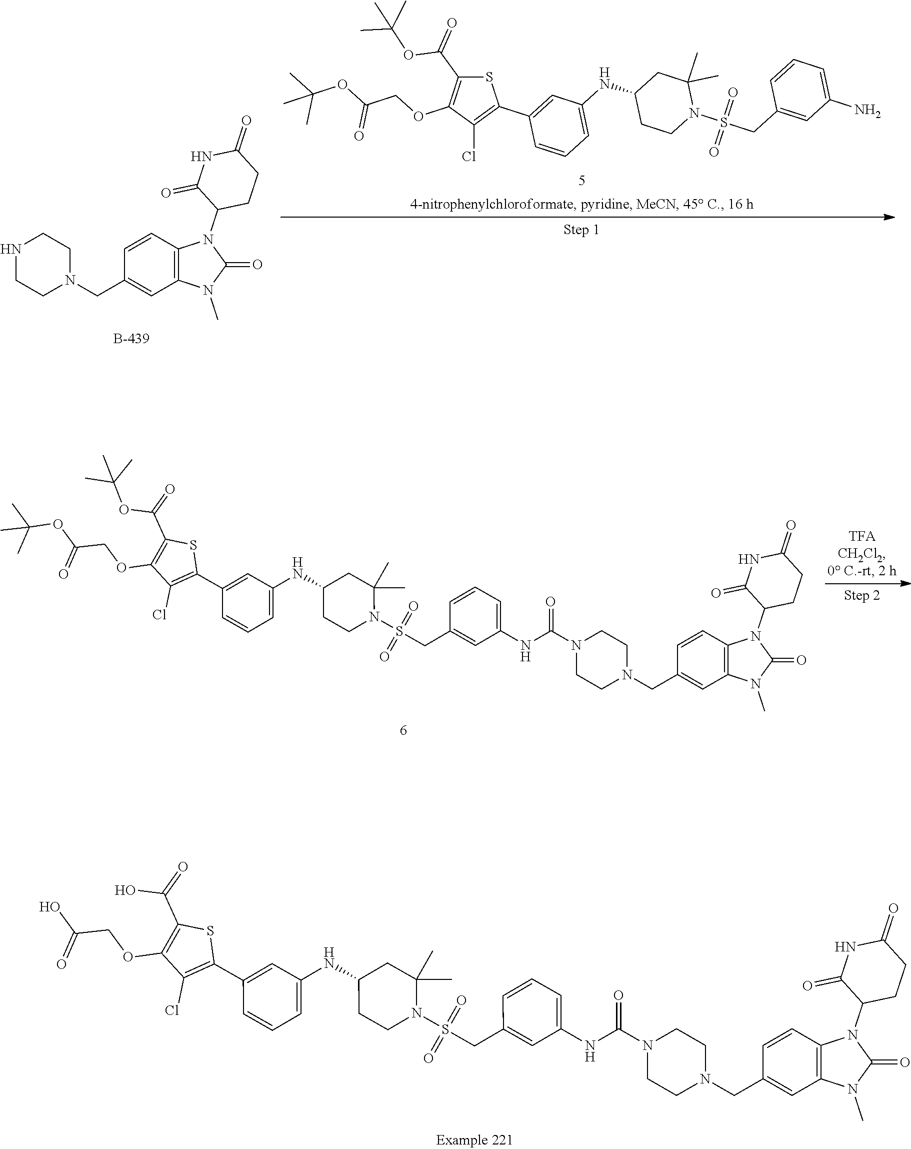

### Step 1: tert-butyl 3-(2-tert-butoxy-2-oxo-ethoxy)-4-chloro-5-[3-[[(4S)-1-[[3-[[4-[[1-(2,6-dioxo-3-piperidyl)-3-methyl-2-oxo-benzimidazol-5-yl]methyl]piperazine-1-carbonyl]amino]phenyl]methylsulfonyl]-2,2-dimethyl-4-piperidyl]amino]phenyl]thiophene-2-carboxylate (6)

tert-butyl 3-(2-tert-butoxy-2-oxo-ethoxy)-4-chloro-5-[3-[[(4S)-1-[3-[4-[1-(2,6-dioxo-3-piperidyl)-3-methyl-2-oxo-benzimidazol-5-yl]methyl]piperazine-1-carbonyl]amino]phenyl]methylsulfonyl]-2,2-dimethyl-4-piperidyl]amino]phenyl]thiophene-2-carboxy late (6, 500 mg, 113.25 μmol, 20% yield, 25% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (5, 410 mg, 569.18 μmol) and 3-[3-methyl-2-oxo-5-(piperazin-1-ylmethyl)benzimidazol-1-yl]piperidine-2,6-dione (B-439, 324.62 mg, 853.77 μmol) in a similar fashion to Example 180. After evaporation of solvent, the residue was treated with water (2 mL). The solid was filtered and dried. LCMS (ES+): m/z 1103.2 [M+H]⁺

### Step 2: 3-(carboxymethoxy)-4-chloro-5-[3-[[(4S)-1-[[3-[[4-[[1-(2,6-dioxo-3-piperidyl)-3-methyl-2-oxo-benzimidazol-5-yl]methyl]piperazine-1-carbonyl]amino]phenyl]methylsulfonyl]-2,2-dimethyl-4-piperidyl]amino]phenyl]thiophene-2-carboxylic acid (221)

3-(carboxymethoxy)-4-chloro-5-[3-[[(4S)-1-[[3-[[4-[1-(2,6-dioxo-3-piperidyl)-3-methyl-2-oxo-benzimidazol-5-yl]methyl]piperazine-1-carbonyl]amino]phenyl]methylsulfonyl]-2,2-dimethyl-4-piperidyl]amino]phenyl]thiophene-2-carboxylic acid (221, 76.92 mg, 69.51 μmol, 40% yield, TFA salt) was synthesized from tert-butyl 3-(2-tert-butoxy-2-oxo-ethoxy)-4-chloro-5-[3-[[(4S)-1-[3-[4-[1-(2,6-dioxo-3-piperidyl)-3-methyl-2-oxo-benzimidazol-5-yl]methyl]piperazine-1-carbonyl]amino]phenyl]methylsulfonyl]-2,2-dimethyl-4-piperidyl]amino]phenyl]thiophene-2-carboxylate (6, 250 mg, 174.50 μmol, TFA salt) in a similar fashion to Compound A-26. The residue was purified by reverse phase prep HPLC [Purification method: Column: XSelect C18 (250×19) mm, 5 micron; Mobile phase A: 0.1% TFA in water; Mobile phase B: MeCN] to afford as an off white solid. LCMS (ES+): m/z 991.0 [M+H]⁺

¹HNMR (400 MHz, DMSO-d6): δ 11.13 (s, 1H), 8.86 (s, 1H), 7.51 (s, 1H), 7.47 (d, J=8.00 Hz, 1H), 7.33 (s, 1H), 7.30-7.18 (m, 4H), 7.03 (d, J=7.60 Hz, 1H), 6.87 (s, 1H), 6.81 (d, J=7.60 Hz, 1H), 6.70-6.68 (m, 1H), 5.45-5.41 (m, 1H), 4.92 (s, 2H), 4.39-4.36 (m, 3H), 4.28-4.25 (m, 2H), 3.65-3.45 (m, 6H), 3.21-3.16 (m, 3H), 2.98-2.82 (m, 1H), 2.75-2.63 (m, 3H), 2.11-1.98 (m, 1H), 1.91-1.72 (m, 2H), 1.49-1.44 (m, 7H), 1.15-1.01 (m, 1H).

### Example 222

### 3-(carboxymethoxy)-4-chloro-5-[3-[[(4S)-1-[[3-[[4-[3-(2,6-dioxo-3-piperidyl)-1-methyl-2-oxo-8,9-dihydro-7H-imidazo[4,5-f]quinolin-6-yl]piperidine-1-carbonyl]amino]phenyl]methylsulfonyl]-2,2-dimethyl-4-piperidyl]amino]phenyl]thiophene-2-carboxylic acid (222)

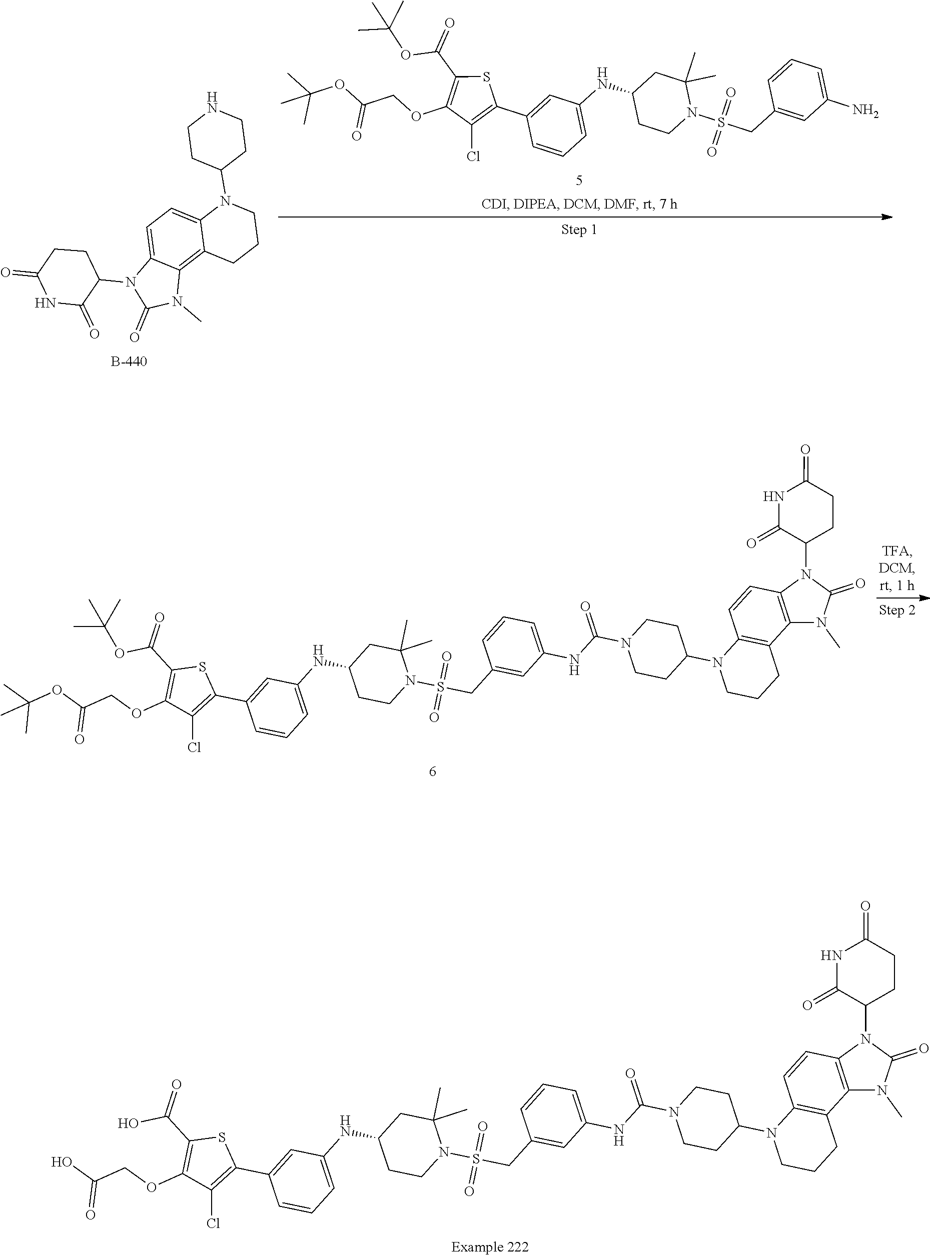

### Step 1; tert-butyl 3-(2-tert-butoxy-2-oxo-ethoxy)-4-chloro-5-[3-[[(4S)-1-[[3-[[4-[3-(2,6-dioxo-3-piperidyl)-1-methyl-2-oxo-8,9-dihydro-7H-imidazo[4,5-f]quinolin-6-yl]piperidine-1-carbonyl]amino]phenyl]methylsulfonyl]-2,2-dimethyl-4-piperidyl]amino]phenyl]thiophene-2-carboxylate (6)

tert-butyl 3-(2-tert-butoxy-2-oxo-ethoxy)-4-chloro-5-[3-[[(4S)-1-[[3-[[4-[3-(2,6-dioxo-3-piperidyl)-1-methyl-2-oxo-8,9-dihydro-7H-imidazo[4,5-f]quinolin-6-yl]piperidine-1-carbonyl]amino]phenyl]methylsulfonyl]-2,2-dimethyl-4-piperidyl]amino]phenyl]thiophene-2-carboxylate (6, 130 mg, 77.51 μmol, 56% yield, 75% purity, TFA salt) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (5, 100 mg, 138.82 μmol) and 3-[1-methyl-2-oxo-6-(4-piperidyl)-8,9-dihydro-7H-imidazo[4,5-f]quinolin-3-yl]piperidine-2,6-dione (B-440, 56.41 mg, 107.88 μmol, TFA salt) in a similar fashion to Compound B-242. The residue was subjected to reverse phase column chromatography [Purification method: Silicycle C18 column; Mobile phase A: 0.1% TFA in water; Mobile phase B: MeCN].

LCMS (ES+): m/z 1143.7 [M+H]⁺

### Step 2: 3-(carboxymethoxy)-4-chloro-5-[3-[[(4S)-1-[3-[4-[3-(2,6-dioxo-3-piperidyl)-1-methyl-2-oxo-8,9-dihydro-7H-imidazo[4,5-f]quinolin-6-yl]piperidine-1-carbonyl]amino]phenyl]methylsulfonyl]-2,2-dimethyl-4-piperidyl]amino]phenyl]thiophene-2-carboxylic acid (222)

3-(carboxymethoxy)-4-chloro-5-[3-[[(4S)-1-[[3-[[4-[3-(2,6-dioxo-3-piperidyl)-1-methyl-2-oxo-8,9-dihydro-7H-imidazo[4,5-f]quinolin-6-yl]piperidine-1-carbonyl]amino]phenyl]methylsulfonyl]-2,2-dimethyl-4-piperidyl]amino]phenyl]thiophene-2-carboxylic acid (222, 75 mg, 64.31 μmol, 83% yield, TFA salt) was synthesized from tert-butyl 3-(2-tert-butoxy-2-oxo-ethoxy)-4-chloro-5-[3-[[(4S)-1-[[3-[[4-[3-(2,6-dioxo-3-piperidyl)-1-methyl-2-oxo-8,9-dihydro-7H-imidazo[4,5-f]quinolin-6-yl]piperidine-1-carbonyl]amino]phenyl]methylsulfonyl]-2,2-dimethyl-4-piperidyl]amino]phenyl]thiophene-2-carboxylate (6, 130 mg, 77.51 μmol, TFA salt) in a similar fashion to Compound A-26. The residue was purified by reverse phase column chromatography [Purification method: Silicycle C18 column; Mobile phase A: 0.1% TFA in water; Mobile phase B: MeCN]. LCMS (ES+): m/z 1031.0 [M+H]⁺

¹HNMR (400 MHz, DMSO-d6): δ 11.08 (s, 1H), 8.64 (s, 1H), 7.55 (s, 1H), 7.45 (d, J=8.16 Hz, 1H), 7.26-7.18 (m, 2H), 6.99 (d, J=7.64 Hz, 1H), 6.87-6.80 (m, 3H), 6.71-6.65 (m, 2H), 5.35-5.25 (m, 1H), 4.92 (s, 2H), 4.38-4.24 (m, 3H), 3.95-3.82 (m, 3H), 3.23-3.10 (m, 4H), 2.92-2.86 (m, 3H), 2.72-0.60 (m, 2H), 1.99-1.60 (m, 8H), 1.49-1.40 (m, 7H), 1.14-1.09 (m, 1H).

### Example 223

### 3-(carboxymethoxy)-4-chloro-5-(3-(((4S)-1-((3-((4S)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylic acid (223)

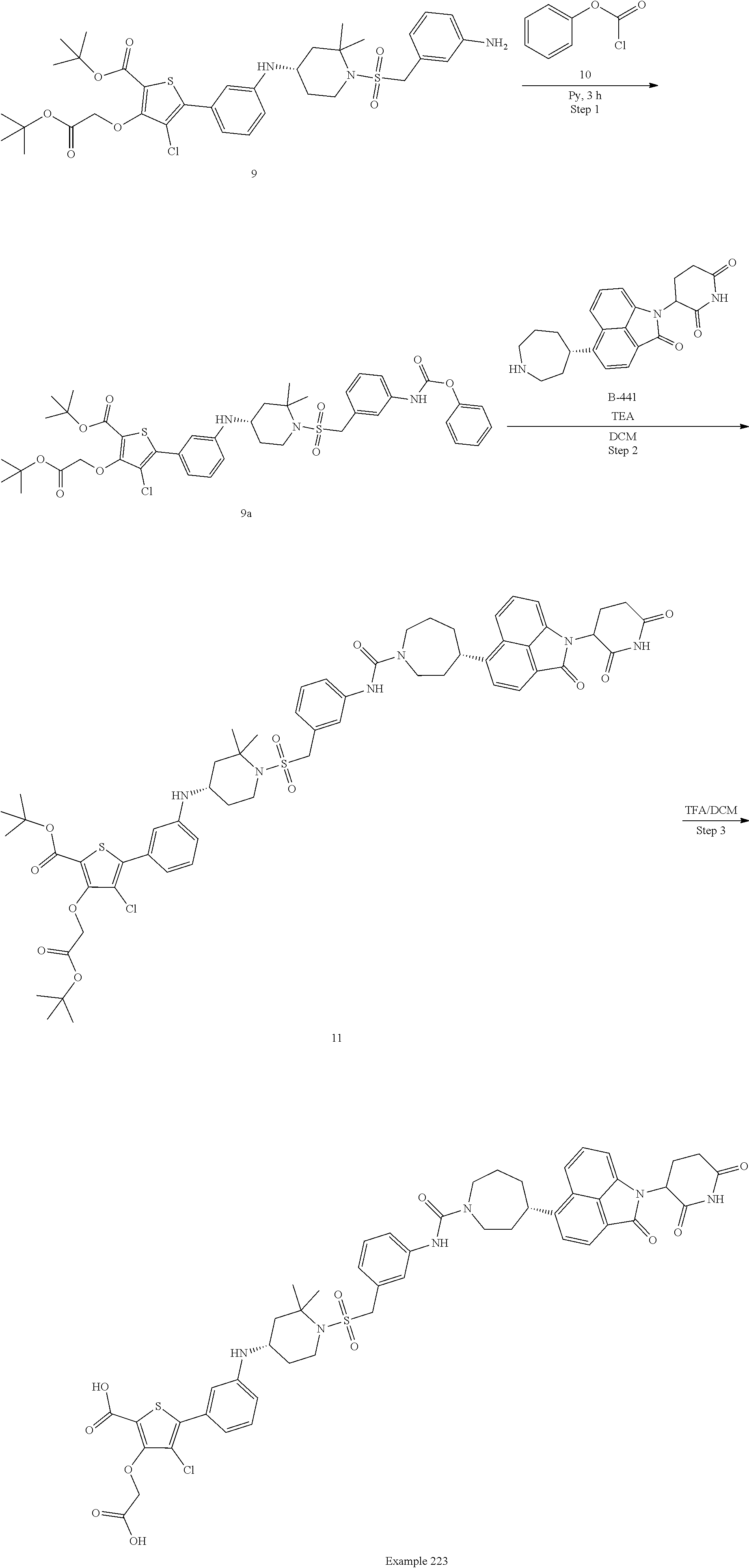

### Step 1: (S)-tert-butyl 3-(2-(tert-butoxy)-2-oxoethoxy)-4-chloro-5-(3-((2,2-dimethyl-1-((3-((phenoxycarbonyl)amino)benzyl)sulfonyl)piperidin-4-yl)amino)phenyl)thiophene-2-carboxylate (9a)

To a solution of(S)-tert-butyl 5-(3-((1-((3-aminobenzyl)sulfonyl)-2,2-dimethylpiperidin-4-yl)amino)phenyl)-3-(2-(tert-butoxy)-2-oxoethoxy)-4-chlorothiophene-2-carboxylate (9, 500 mg, 694.12 μmol) in Py (1 mL) was added phenyl carbonochloridate (10, 130.41 mg, 832.94 μmol, 104.33 μL) at 0° C. . . . The mixture was stirred at 20° C. for 3 h. The reaction mixture was filtered and concentrated under reduced pressure. The residue was purified by column chromatography (SiO₂, Petroleum ether:Ethyl acetate=1:0 to 1:1) to afford(S)-tert-butyl 3-(2-(tert-butoxy)-2-oxoethoxy)-4-chloro-5-(3-((2,2-dimethyl-1-((3-((phenoxycarbonyl)amino)benzyl)sulfonyl)piperidin-4-yl)amino)phenyl)thiophene-2-carboxylate (9a, 300 mg, 356.95 μmol, 51% yield) as a yellow solid. LCMS (ESI): m/z 840.4 [M+H]⁺

### Step 2: tert-butyl 3-(2-(tert-butoxy)-2-oxoethoxy)-4-chloro-5-(3-(((4S)-1-((3-((4S)-4-(1-(2,6-dioxopiperidin-3-yl)-2-oxo-1,2-dihydrobenzo (cd]indol-5-yl) azepane-1-carboxamido)benzyl)sulfonyl)-2,2-dimethylpiperidin-4-yl)amino)phenyl)thiophene-2-carboxylate (11)

To a solution of 3-(5-((S)-azepan-4-yl)-2-oxobenzo[cd]indol-[(2H)-yl)piperidine-2,6-dione (B-441, 30 mg, 72.48 μmol) in DMF (1 mL) and DCM (1 mL) was added Et₃N (18.34 mg, 181.20 μmol, 25.26 μL), then the mixture was added(S)-tert-butyl 3-(2-(tert-butoxy)-2-oxoethoxy)-4-chloro-5-(3-((2,2-dimethyl-1-((3-((phenoxycarbonyl)amino)benzyl)sulfonyl)piperidin-4-yl)amino)phenyl)thiophene-2-carboxylate (9a, 60.92 mg, 72.48 μmol) at 0° C. The mixture was stirred at 30° C. for 16 h. The reaction mixture was filtered and concentrated under reduced pressure. The residue purified by column chromatography (SiO₂, Petroleum ether:Ethyl acetate=1:0 to 1:1) to afford tert-butyl 3-(2-(tert-butoxy)-2-oxoethoxy)-4-chloro-5-(3-(((4S)-1-((3-((4S)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylate (11, 56 mg, 49.83 μmol, 69% yield) as a white solid. LCMS (ESI): m/z 1123.8 [M+H]⁺

### Step 3: 3-(carboxymethoxy)-4-chloro-5-(3-(((4S)-1-((3-((4S)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylic acid (223)

3-(carboxymethoxy)-4-chloro-5-(3-(((4S)-1-((3-((4S)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylic acid (223, 14.57 mg, 12.82 μmol, 26% yield, TFA salt) was synthesized from tert-butyl 3-(2-tert-butoxy-2-oxo-ethoxy)-4-chloro-5-[3-[[(4S)-1-[[3-[[(4S)-4-[1-(2,6-dioxo-3-piperidyl)-2-oxo-benzo[cd]indol-5-yl]azepane-1-carbonyl]amino]phenyl]methylsulfonyl]-2,2-dimethyl-4-piperidyl]amino]phenyl]thiophene-2-carboxylate (11, 56.00 mg, 49.83 μmol) in a similar fashion to Compound A-26. The residue was purified by prep-HPLC (flow: 25 mL/min; gradient: from 75%-45% water (0.1% TFA)-ACN; column: Phenomenex Luna C18 150× 25 mm×10 μm). LCMS (ESI): m/z 708.2 [M+H]⁺

¹H NMR (400 MHz, DMSO-d6) δ 11.11 (s, 1H), 8.38 (s, 1H), 8.06-7.98 (m, 1H), 7.81 (d, J=8.8 Hz, 1H), 7.69-7.62 (m, 2H), 7.55-7.49 (m, 2H), 7.26 (t, J=7.9 Hz, 1H), 7.20-7.12 (m, 2H), 7.01 (br d, J=7.9 Hz, 1H), 6.86 (s, 1H), 6.81-6.76 (m, 1H), 6.67 (br d, J=8.1 Hz, 1H), 5.50-5.37 (m, 1H), 4.91 (s, 2H), 4.39-4.24 (m, 2H), 3.92-3.78 (m, 2H), 3.28-3.09 (m, 4H), 3.06-2.86 (m, 2H), 2.82-2.67 (m, 2H), 2.09-1.88 (m, 8H), 1.78 (br d, J=11.5 Hz, 1H), 1.51-1.47 (m, 3H), 1.43 (br s, 1H), 1.40 (s, 3H), 1.23-1.05 (m, 2H).

### Example 224

### 3-(carboxymethoxy)-4-chloro-5-(3-(((4S)-1-((3-((4R)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylic acid (224)

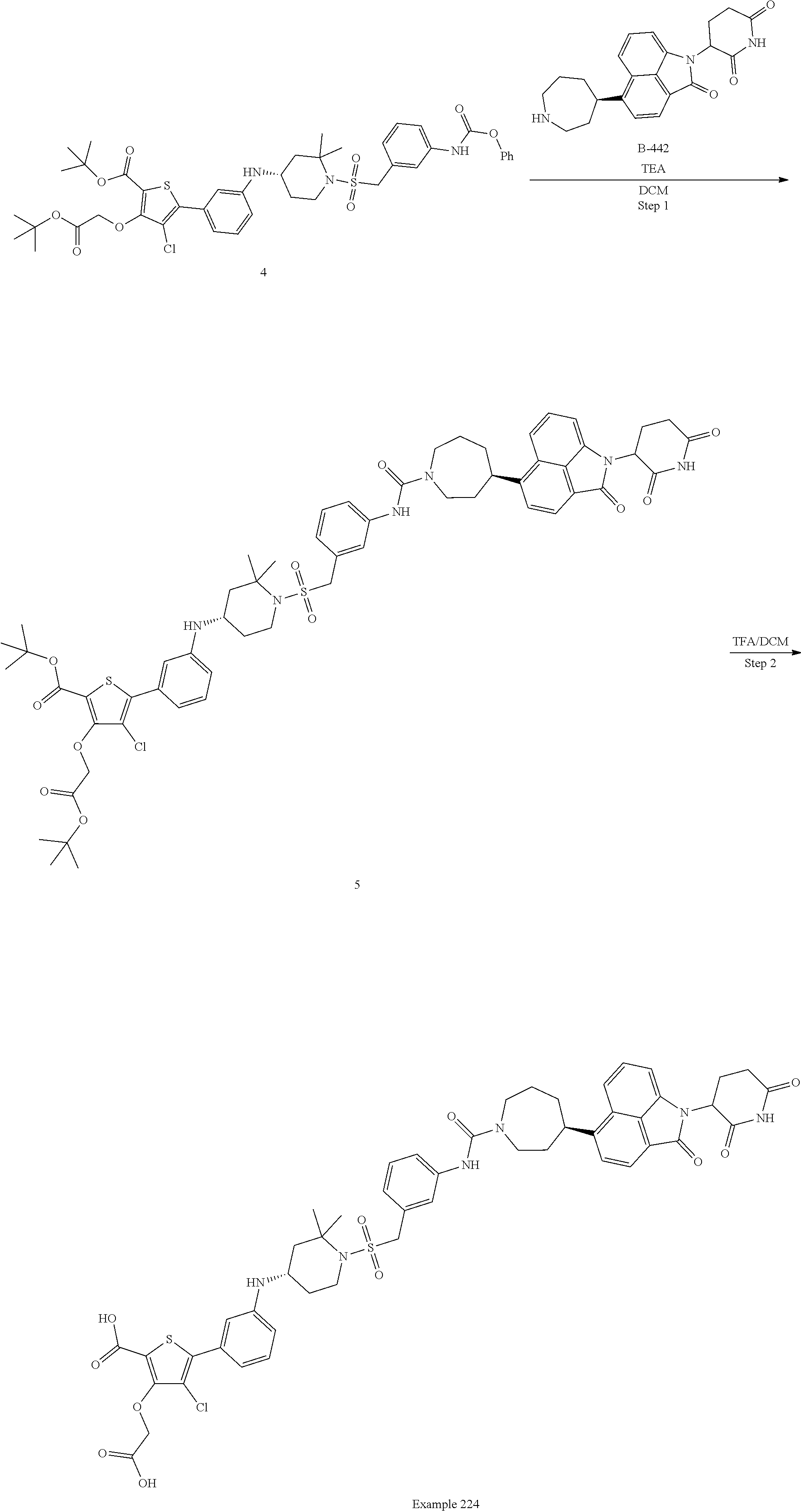 [SMILES: CC(C)(C)OC(=O)COc1c(C(=O)OC(C)(C)C)sc(-c2cccc(N[C@H]3CCN(S(=O)(=O)Cc4cccc(NC(=O)Oc5ccccc5)c4)C(C)(C)C3)c2)c1Cl ; O=C1CCC(N2C(=O)c3ccc([C@@H]4CCCNCC4)c4cccc2c34)C(=O)N1 ; CC(C)(C)OC(=O)COc1c(C(=O)OC(C)(C)C)sc(-c2cccc(N[C@H]3CCN(S(=O)(=O)Cc4cccc(NC(=O)N5CCC[C@@H](c6ccc7c8c(cccc68)N(C6CCC(=O)NC6=O)C7=O)CC5)c4)C(C)(C)C3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)N2CCC[C@@H](c3ccc4c5c(cccc35)N(C3CCC(=O)NC3=O)C4=O)CC2)c1]

### Step 1: tert-butyl 3-(2-(tert-butoxy)-2-oxoethoxy)-4-chloro-5-(3-(((4S)-1-((3-((4R)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylate (5)

To a solution of 3-(5-((R)-azepan-4-yl)-2-oxobenzo[cd]indol-[(2H)-yl)piperidine-2,6-dione (B-442, 36.48 mg, 96.64 μmol, HCl salt) in DMF (1 mL) and DCM (1 mL) was added TEA (24.45 mg, 241.61 mol, 33.68 μL). Then tert-butyl 3-(2-tert-butoxy-2-oxo-ethoxy)-4-chloro-5-[3-[[(4S)-2,2-dimethyl-1-[[3-(phenoxycarbonylamino)phenyl]methylsulfonyl]-4-piperidyl]amino]phenyl]thiophene-2-carboxylate (4, 81.22 mg, 96.64 μmol) was added at 0° C. The mixture was stirred at 30° C. for 16 h. The reaction mixture was filtered and concentrated under reduced pressure. The residue purified by column chromatography (SiO2, Petroleum ether:Ethyl acetate=1:0 to 1:1) to afford tert-butyl 3-(2-(tert-butoxy)-2-oxoethoxy)-4-chloro-5-(3-(((4S)-1-((3-((4R)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylate (5, 75 mg, 66.74 μmol, 69% yield) as a white solid.

LCMS (ESI): m/z 1123.8 [M+H]⁺

### Step 2: 3-(carboxymethoxy)-4-chloro-5-(3-(((4S)-1-((3-((4R)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylic acid (224)

3-(carboxymethoxy)-4-chloro-5-(3-(((4S)-1-((3-((4R)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylic acid (224, 6.03 mg, 5.25 μmol, 8% yield, TFA salt) was synthesized from tert-butyl 3-(2-(tert-butoxy)-2-oxoethoxy)-4-chloro-5-(3-(((4S)-1-((3-((4R)-4-(1-(2,6-dioxopiperidin-3-yl)-2-oxo-1,2-dihydrobenzo[cd]indol-5-yl) azepane-1-carboxamido)benzyl)sulfonyl)-2,2-dimethylpiperidin-4-yl)amino)phenyl)thiophene-2-carboxylate (5, 74 mg, 65.85 μmol) in a similar fashion to Compound A-26. The residue was purified by prep-HPLC (flow: 25 mL/min; gradient: from 75%-45% water (0.1% TFA)-ACN; column: Phenomenex Luna C18 150×25 mm×10 μm). LCMS (ESI): m/z 1011.5 [M+H]⁺

¹H NMR (400 MHz, DMSO-d₆) δ 11.11 (s, 1H), 8.38 (s, 1H), 8.03-7.99 (m, 1H), 7.83-7.79 (m, 1H), 7.69-7.64 (m, 2H), 7.54-7.50 (m, 2H), 7.29-7.24 (m, 1H), 7.20-7.12 (m, 2H), 7.01 (d, J=7.6 Hz, 1H), 6.87-6.85 (m, 1H), 6.81-6.76 (m, 1H), 6.70-6.65 (m, 1H), 5.46-5.41 (m, 1H), 4.92-4.90 (m, 2H), 4.37-4.27 (m, 2H), 3.89 (br d, J=4.4 Hz, 2H), 3.48 (br dd, J=3.4, 14.2 Hz, 5H), 3.23-3.03 (m, 2H), 2.97-2.90 (m, 1H), 2.80-2.73 (m, 1H), 2.08-1.86 (m, 8H), 1.78 (br d, J=12.0 Hz, 1H), 1.49 (s, 3H), 1.45 (br s, 1H), 1.40 (s, 3H), 1.16-1.10 (m, 1H).

### Example 225

### 3-(carboxymethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)oxy)phenyl)thiophene-2-carboxylic acid (225)

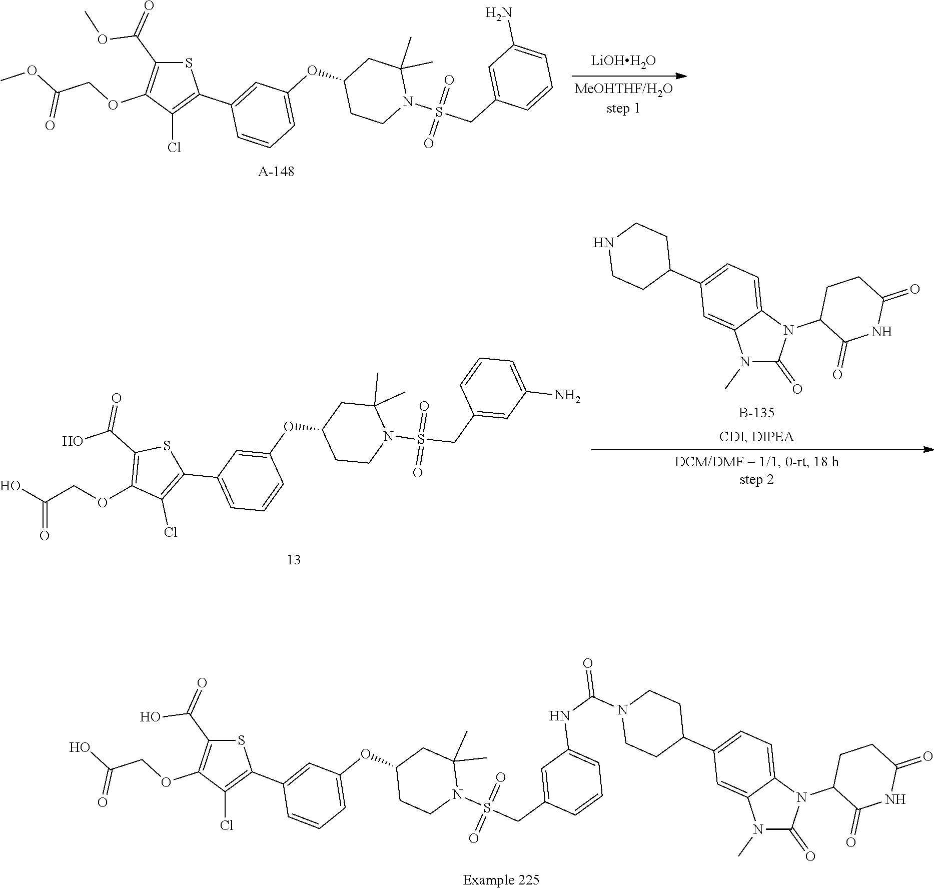 [SMILES: COC(=O)COc1c(C(=O)OC)sc(-c2cccc(O[C@H]3CCN(S(=O)(=O)Cc4cccc(N)c4)C(C)(C)C3)c2)c1Cl ; CC1(C)C[C@@H](Oc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)Nc4cccc(CS(=O)(=O)N5CC[C@H](Oc6cccc(-c7sc(C(=O)O)c(OCC(=O)O)c7Cl)c6)CC5(C)C)c4)CC3)cc21]

### Step 1: (S)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (13)

(S)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (13, 130 mg, 211.29 μmol, 67% yield) was synthesized from(S)-methyl 5-(3-((1-((3-aminobenzyl)sulfonyl)-2,2-dimethylpiperidin-4-yl)oxy)phenyl)-4-chloro-3-(2-methoxy-2-oxoethoxy)thiophene-2-carboxylate (A-148, 200 mg, 313.89 μmol) in a similar fashion to Compound A-9 using MeOH/THE/water (2:2:1) as the solvent. The mixture was adjusted pH to 4 with 1 N HCl aqueous and then lyophilized. The residue was purified by prep-HPLC (flow: 25 mL/min; gradient: from 32-62% MeCN in water (0.1% TFA) over 7 min; column: 3_Phenomenex luna C18 75×30 mm×3 μm). LCMS (ESI): m/z 609.2 [M+H]+

### Step 2: 3-(carboxymethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)oxy)phenyl)thiophene-2-carboxylic acid (225)

3-(carboxymethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)oxy)phenyl)thiophene-2-carboxylic acid (225, 15 mg, 13.60 μmol, 17% yield, TFA salt) was synthesized from(S)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (13, 50 mg, 82.09 μmol) in DCM (1 mL) and 3-(3-methyl-2-oxo-5-(piperidin-4-yl)-2,3-dihydro-1H-benzo[d]imidazol-1-yl)piperidine-2,6-dione (B-135, 28.11 mg, 82.09 μmol) in a similar fashion to Compound B-242. The mixture was purified by prep-HPLC (flow: 25 mL/min; gradient: from 47-77% MeCN in water (0.1% TFA) over 10 min; column: Phenomenex Synergi C18 150×25 mm×10 μm). LCMS (ESI): m/z 977.3 [M+H]+

1H NMR (400 MHz, DMSO-d₆) δ 11.08 (s, 1H), 8.62 (s, 1H), 7.63 (s, 1H), 7.49 (d, J=8.0 Hz, 1H), 7.44-7.37 (m, 1H), 7.28-7.17 (m, 3H), 7.10 (s, 1H), 7.06 (d, J=9.2 Hz, 1H), 6.99 (t, J=8.4 Hz, 2H), 6.92 (d, J=8.0 Hz, 1H), 5.33 (dd, J=5.2, 12.8 Hz, 1H), 4.90 (s, 2H), 4.72-4.60 (m, 1H), 4.41-4.21 (m, 4H), 3.45-3.40 (m, 1H), 3.32 (s, 3H), 3.21-3.14 (m, 1H), 2.95-2.82 (m, 3H), 2.64-2.59 (m, 1H), 2.02-1.98 (m, 1H), 1.96-1.86 (m, 2H), 1.84-1.75 (m, 2H), 1.72-1.57 (m, 4H), 1.51 (s, 3H), 1.41 (s, 3H), 1.36-1.22 (m, 2H).

### Example 226

### 3-(carboxymethoxy)-4-chloro-5-(3-(((4R)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yloxy)phenyl)thiophene-2-carboxylic acid (226)

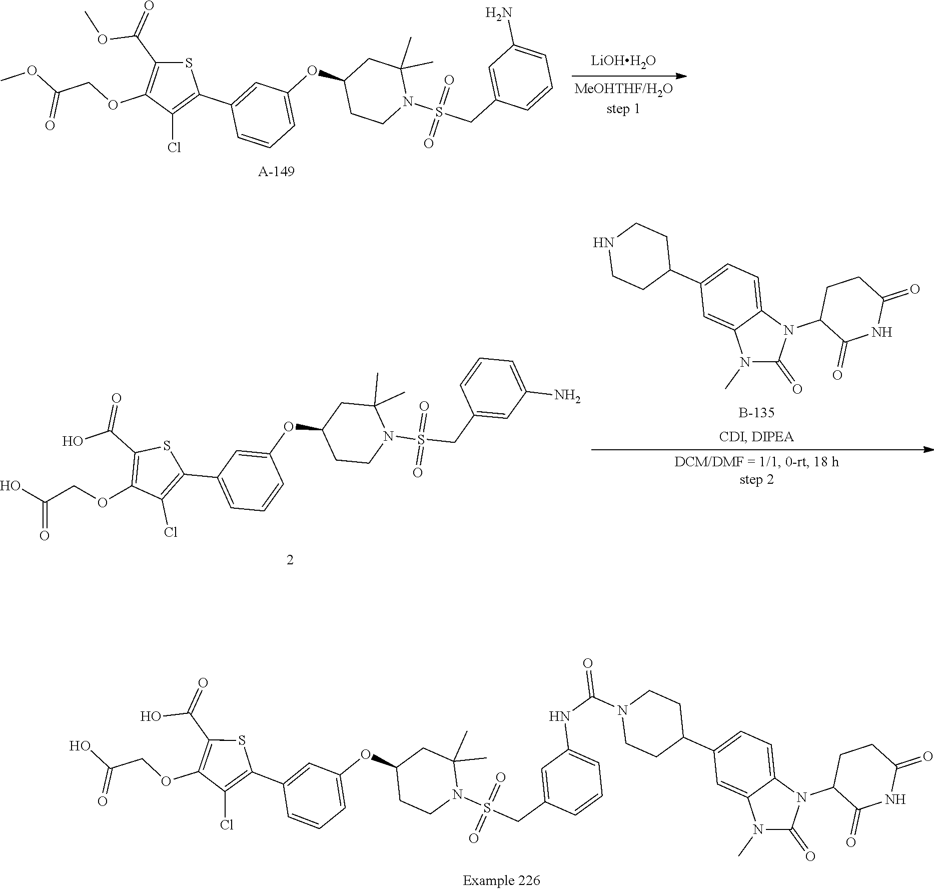 [SMILES: COC(=O)COc1c(C(=O)OC)sc(-c2cccc(O[C@@H]3CCN(S(=O)(=O)Cc4cccc(N)c4)C(C)(C)C3)c2)c1Cl ; CC1(C)C[C@H](Oc2cccc(-c3sc(C(=O)O)c(OCC(=O)O)c3Cl)c2)CCN1S(=O)(=O)Cc1cccc(N)c1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)Nc4cccc(CS(=O)(=O)N5CC[C@@H](Oc6cccc(-c7sc(C(=O)O)c(OCC(=O)O)c7Cl)c6)CC5(C)C)c4)CC3)cc21]

### Step 1: (R)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (2)

(R)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (2, 210 mg, 324.08 μmol) was synthesized from (R)-methyl 5-(3-((1-(3-aminobenzyl)sulfonyl)-2,2-dimethylpiperidin-4-yloxy phenyl)-4-chloro-3-(2-methoxy-2-oxoethoxy)thiophene-2-carboxylate (A-149, 200 mg, 313.89 μmol) in a similar fashion to Compound A-9 using MeOH/THF/water (2:2:1) as the solvent. The mixture was adjusted pH to 4 with 1 N HCl aqueous and then lyophilized. The residue was combined with another batch (100 mg) and purified by prep-HPLC (flow: 25 mL/min; gradient: from 32-62% MeCN in water (0.1% TFA) over 7 min; column: 3_Phenomenex luna C18 75×30 mm×3 μm).

LCMS (ESI): m/z 609.1 [M+H]⁺

### Step 2: 3-(carboxymethoxy)-4-chloro-5-(3-(((4R)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)oxy)phenyl)thiophene-2-carboxylic acid (226)

3-(carboxymethoxy)-4-chloro-5-(3-(((4R)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)oxy)phenyl)thiophene-2-carboxylic acid (226, 15 mg, 13.60 μmol, 14% yield, TFA salt) was synthesized from (R)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (2, 60 mg, 98.50 μmol) and 3-(3-methyl-2-oxo-5-(piperidin-4-yl)-2,3-dihydro-1H-benzo[d]imidazol-1-yl)piperidine-2,6-dione (B-135, 33.73 mg, 98.50 μmol) in a similar fashion to Compound B-242. The mixture was purified by prep-HPLC (flow: 25 mL/min; gradient: from 44-74% MeCN in water (0.1% TFA) over 10 min; column: 3_Phenomenex luna C18 75×30 mm×3 μm).

LCMS (ESI): m/z 977.2 [M+H]⁺

¹H NMR (400 MHz, DMSO-d₆) δ 11.08 (s, 1H), 8.62 (s, 1H), 7.63 (s, 1H), 7.49 (d, J=8.8 Hz, 1H), 7.45-7.37 (m, 1H), 7.28-7.19 (m, 3H), 7.10 (s, 1H), 7.06 (dd, J=1.2, 8.4 Hz, 1H), 7.02-6.96 (m, 2H), 6.92 (d, J=7.6 Hz, 1H), 5.33 (dd, J=4.8, 12.4 Hz, 1H), 4.99-4.86 (m, 2H), 4.66 (dt, J=4.4, 8.4 Hz, 1H), 4.40-4.23 (m, 4H), 3.55-3.48 (m, 1H), 3.32 (s, 3H), 3.21-3.15 (m, 1H), 2.92-2.86 (m, 3H), 2.67-2.66 (m, 1H), 2.04-1.97 (m, 1H), 1.92 (d, J=10.8 Hz, 2H), 1.80 (d, J=12.4 Hz, 3H), 1.72-1.58 (m, 4H), 1.51 (s, 3H), 1.41 (s, 3H), 1.35-1.23 (m, 2H).

### Example 227

### 3-(carboxymethoxy)-4-chloro-5-(3-(((4R)-1-((3-(4-(1-(2,6-dioxopiperidin-3-yl)-2-oxo-1,2-dihydrobenzo[cd]indol-6-yl)piperidine-1-carboxamido)benzyl)sulfonyl)-2,2-dimethylpiperidin-4-yl)oxy)phenyl)thiophene-2-carboxylic acid (227)

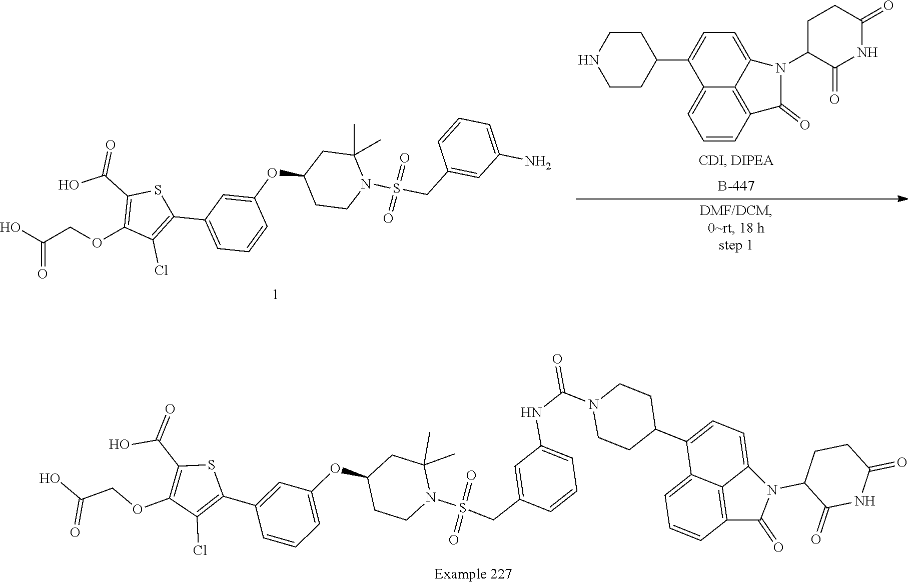 [SMILES: CC1(C)C[C@H](Oc2cccc(-c3sc(C(=O)O)c(OCC(=O)O)c3Cl)c2)CCN1S(=O)(=O)Cc1cccc(N)c1 ; O=C1CCC(N2C(=O)c3cccc4c(C5CCNCC5)ccc2c34)C(=O)N1 ; CC1(C)C[C@H](Oc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1]

### Step 1: 3-(carboxymethoxy)-4-chloro-5-(3-(((4R)-1-((3-(4-(1-(2,6-dioxopiperidin-3-yl)-2-oxo-1,2-dihydrobenzo[cd]indol-6-yl)piperidine-1-carboxamido)benzyl)sulfonyl)-2,2-dimethylpiperidin-4-yloxy)phenyl)thiophene-2-carboxylic acid (227)

3-(carboxymethoxy)-4-chloro-5-(3-(((4R)-1-((3-(4-(1-(2,6-dioxopiperidin-3-yl)-2-oxo-1,2-dihydrobenzo[cd]indol-6-yl)piperidine-1-carboxamido)benzyl)sulfonyl)-2,2-dimethylpiperidin-4-yl)oxy)phenyl)thiophene-2-carboxylic acid (227, 22 mg, 19.58 μmol, 15% yield, TFA salt) was synthesized from (R)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (1, 80 mg, 131.34 μmol) and 3-(2-oxo-6-(piperidin-4-yl)benzo[cd]indol-[(2H)-yl)piperidine-2,6-dione (B-447, 62.71 mg, 131.34 μmol, TFA salt) in a similar fashion to Compound B-242. The mixture was purified by prep-HPLC (flow: 25 mL/min; gradient: from 48-78% MeCN in water (0.1% TFA) over 7 min; column: 3_Phenomenex luna C18 75×30 mm×3 μm).

LCMS (ESI): m/z 998.3 [M+H]+

¹H NMR (400 MHz, DMSO-d₆) δ 11.11 (s, 1H), 8.67 (s, 1H), 8.49 (d, J=8.4 Hz, 1H), 8.10 (d, J=6.8 Hz, 1H), 7.87 (dd, J=7.2, 8.0 Hz, 1H), 7.64 (s, 1H), 7.50 (d, J=8.8 Hz, 1H), 7.45-7.34 (m, 2H), 7.29-7.17 (m, 3H), 7.12-7.03 (m, 2H), 6.98 (d, J=6.8 Hz, 1H), 5.50-5.38 (m, 1H), 4.90 (s, 2H), 4.71-4.59 (m, 1H), 4.44-4.22 (m, 4H), 3.64-3.50 (m, 1H), 3.23-3.15 (m, 1H), 3.12-3.02 (m, 2H), 2.98-2.89 (m, 1H), 2.79-2.71 (m, 1H), 2.62-2.60 (m, 1H), 2.11-2.03 (m, 1H), 1.97-1.87 (m, 4H), 1.82-1.61 (m, 3H), 1.59-1.46 (m, 3H), 1.41 (s, 3H), 1.37-1.23 (m, 2H).

### Example 228

### 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)benzyl)sulfonyl)-2,2-dimethylpiperidin-4-yloxy)phenyl)thiophene-2-carboxylic acid (228)

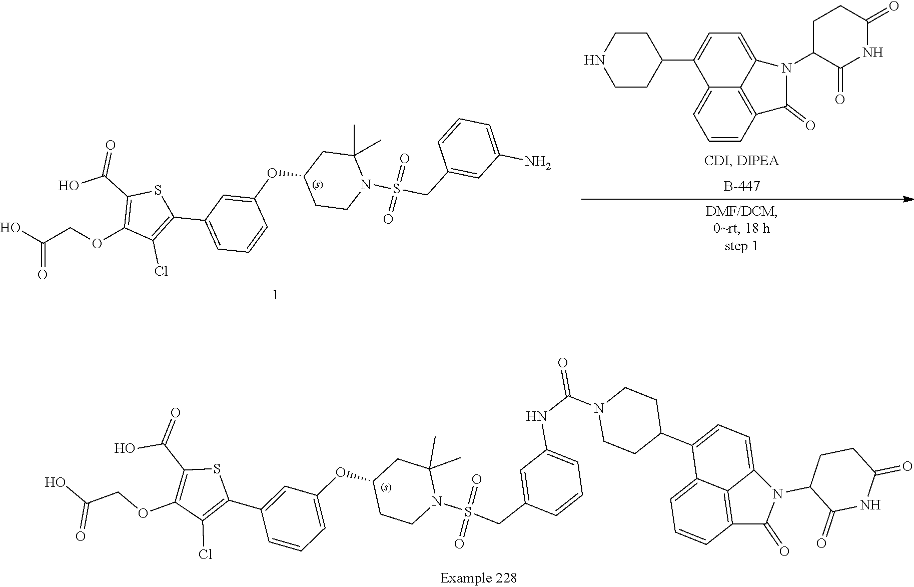 [SMILES: CC1(C)C[C@@H](Oc2cccc(-c3sc(C(=O)O)c(OCC(=O)O)c3Cl)c2)CCN1S(=O)(=O)Cc1cccc(N)c1 ; O=C1CCC(N2C(=O)c3cccc4c(C5CCNCC5)ccc2c34)C(=O)N1 ; CC1(C)C[C@@H](Oc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1]

### Step 1: 3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)benzyl)sulfonyl)-2,2-dimethylpiperidin-4-yloxy)phenyl)thiophene-2-carboxylic acid (228)

3-(carboxymethoxy)-4-chloro-5-(3-(((4S)-1-((3-(4-(1-(2,6-dioxopiperidin-3-yl)-2-oxo-1,2-dihydrobenzo[cd]indol-6-yl)piperidine-1-carboxamido)benzyl)sulfonyl)-2,2-dimethylpiperidin-4-yl)oxy)phenyl)thiophene-2-carboxylic acid (228, 10 mg, 8.90 μmol, 6% yield, TFA salt) was synthesized from(S)-5-(3-((1-((3-aminobenzyl)sulfonyl)-2,2-dimethylpiperidin-4-yl)oxy)phenyl)-3-(carboxymethoxy)-4-chlorothiophene-2-carboxylic acid (1, 88 mg, 144.47 μmol) and DIPEA (93.36 mg, 722.37 μmol, 125.82 μL) and 3-(2-oxo-6-(piperidin-4-yl)benzo[cd]indol-[(2H)-yl)piperidine-2,6-dione (B-447, 68.98 mg, 144.47 μmol, TFA salt) in a similar fashion to Compound B-242. The mixture was purified by prep-HPLC (flow: 25 mL/min; gradient: from 48-78% MeCN in water (0.1% TFA) over 7 min; column: 3_Phenomenex luna C18 75×30 mm×3 μm).

LCMS (ESI): m/z 998.2 [M+H]+

¹H NMR (400 MHz, DMSO-d₆) δ 11.11 (s, 1H), 8.67 (s, 1H), 8.49 (d, J=8.4 Hz, 1H), 8.10 (d, J=6.8 Hz, 1H), 7.90-7.82 (m, 1H), 7.64 (s, 1H), 7.53-7.47 (m, 1H), 7.43-7.35 (m, 2H), 7.28-7.17 (m, 3H), 7.10-7.02 (m, 2H), 7.01-6.95 (m, 1H), 5.50-5.37 (m, 1H), 4.89 (s, 2H), 4.73-4.60 (m, 1H), 4.43-4.23 (m, 4H), 3.65-3.52 (m, 1H), 3.48-3.39 (m, 1H), 3.24-3.12 (m, 1H), 3.11-3.00 (m, 1H), 2.99-2.88 (m, 1H), 2.79-2.70 (m, 1H), 2.65-2.59 (m, 1H), 2.15-2.02 (m, 1H), 1.98-1.85 (m, 4H), 1.83-1.62 (m, 3H), 1.51 (s, 3H), 1.41 (s, 3H), 1.37-1.23 (m, 2H).

### Example 229

### 3-(carboxymethoxy)-4-chloro-5-(3-(((75)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylic acid (229)

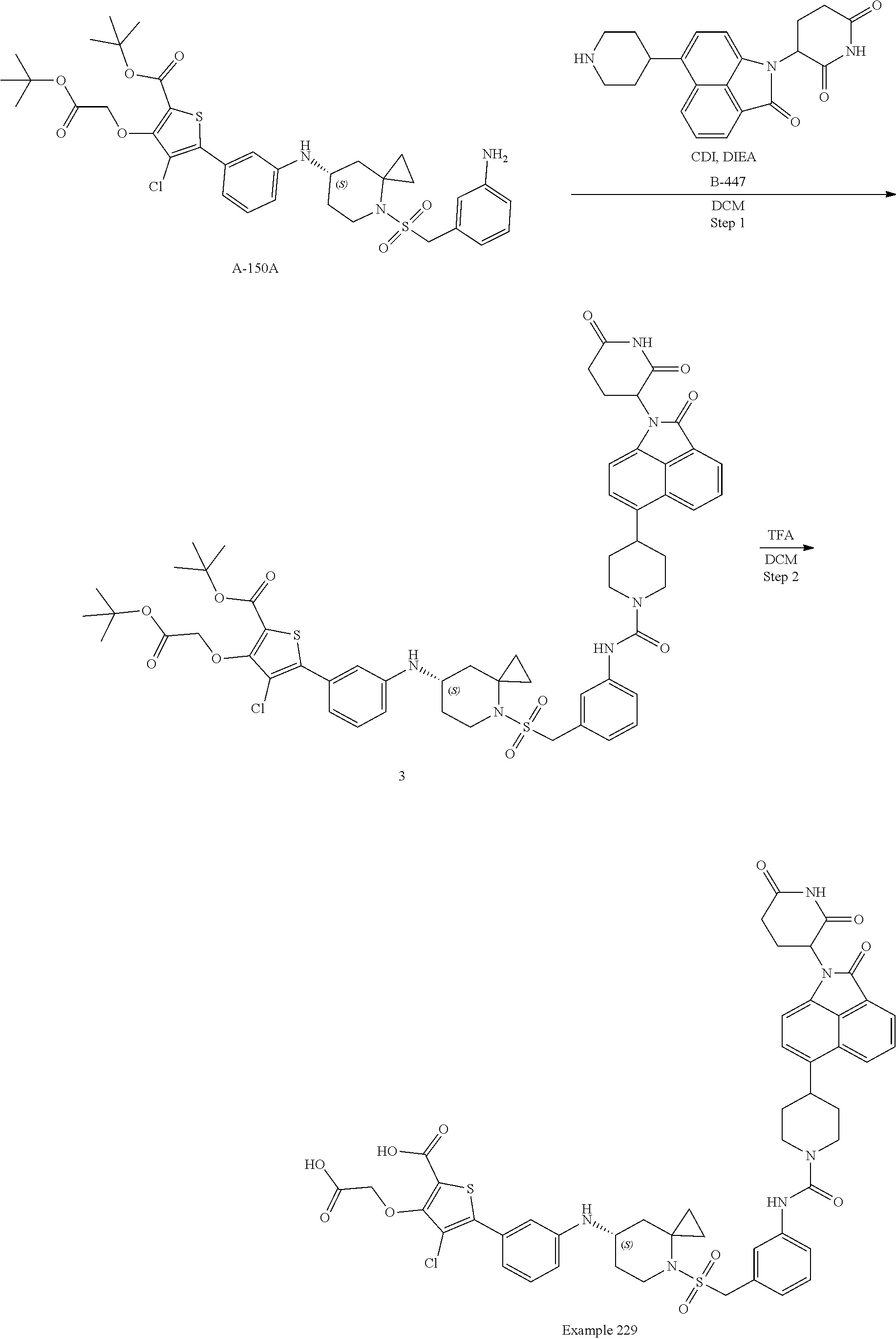

### Step 1: tert-butyl 3-(2-(tert-butoxy)-2-oxoethoxy)-4-chloro-5-(3-(((7S)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylate (3)

tert-butyl 3-(2-(tert-butoxy)-2-oxoethoxy)-4-chloro-5-(3-(((75)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylate (3, 100 mg, 90.28 μmol, 65% yield) was synthesized from(S)-tert-butyl 5-(3-((4-((3-aminobenzyl)sulfonyl)-4-azaspiro[2.5]octan-7-yl)amino)phenyl)-3-(2-(tert-butoxy)-2-oxoethoxy)-4-chlorothiophene-2-carboxylate (A-150A, 100 mg, 139.21 μmol) and 3-(2-oxo-6-(piperidin-4-yl)benzo[cd]indol-[(2H)-yl)piperidine-2,6-dione (B-447, 55.67 mg, 139.21 μmol) in a similar fashion to Compound B-242. The residue was purified by column chromatography (SiO₂, Petroleum ether:Ethyl acetate=1:0 to 0:1). LCMS (ESI): m/z 1107.6 [M+H]⁺

### Step 2: 3-(carboxymethoxy)-4-chloro-5-(3-(((7S)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylic acid (229)

3-(carboxymethoxy)-4-chloro-5-(3-(((75)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylic acid (229, 24.25 mg, 21.42 μmol, 26% yield, TFA salt) was synthesized from tert-butyl 3-(2-(tert-butoxy)-2-oxoethoxy)-4-chloro-5-(3-(((75)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylate (3, 90 mg, 81.25 μmol) in a similar fashion to Compound A-26. The residue was purified by prep-HPLC (flow: 25 mL/min; gradient: from 72%-42% water (0.1% TFA)-ACN; column: 3_Phenomenex Luna C18 150×25 mm×10 μm). LCMS (ESI): m/z 999.5 [M+H]⁺

¹H NMR (400 MHz, DMSO-d₆) δ 11.12 (s, 1H), 8.68-8.61 (m, 1H), 8.51 (d, J=8.4 Hz, 1H), 8.14-8.09 (m, 1H), 7.92-7.85 (m, 1H), 7.49-7.45 (m, 2H), 7.42-7.37 (m, 1H), 7.23-7.15 (m, 2H), 7.13-7.07 (m, 1H), 6.88-6.84 (m, 2H), 6.82-6.77 (m, 1H), 6.72-6.66 (m, 1H), 5.44 (br dd, J=5.0, 13.2 Hz, 1H), 4.91 (s, 2H), 4.35-4.18 (m, 4H), 3.61-3.55 (m, 3H), 3.10-3.01 (m, 3H), 2.99-2.94 (m, 1H), 2.80-2.72 (m, 2H), 2.11-2.06 (m, 1H), 2.02-1.87 (m, 3H), 1.82-1.64 (m, 3H), 1.60-1.45 (m, 1H), 1.38-1.28 (m, 1H), 0.68-0.60 (m, 2H), 0.57-0.45 (m, 2H).

### Example 230

### 3-(carboxymethoxy)-4-chloro-5-(3-(((7R)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylic acid (230)

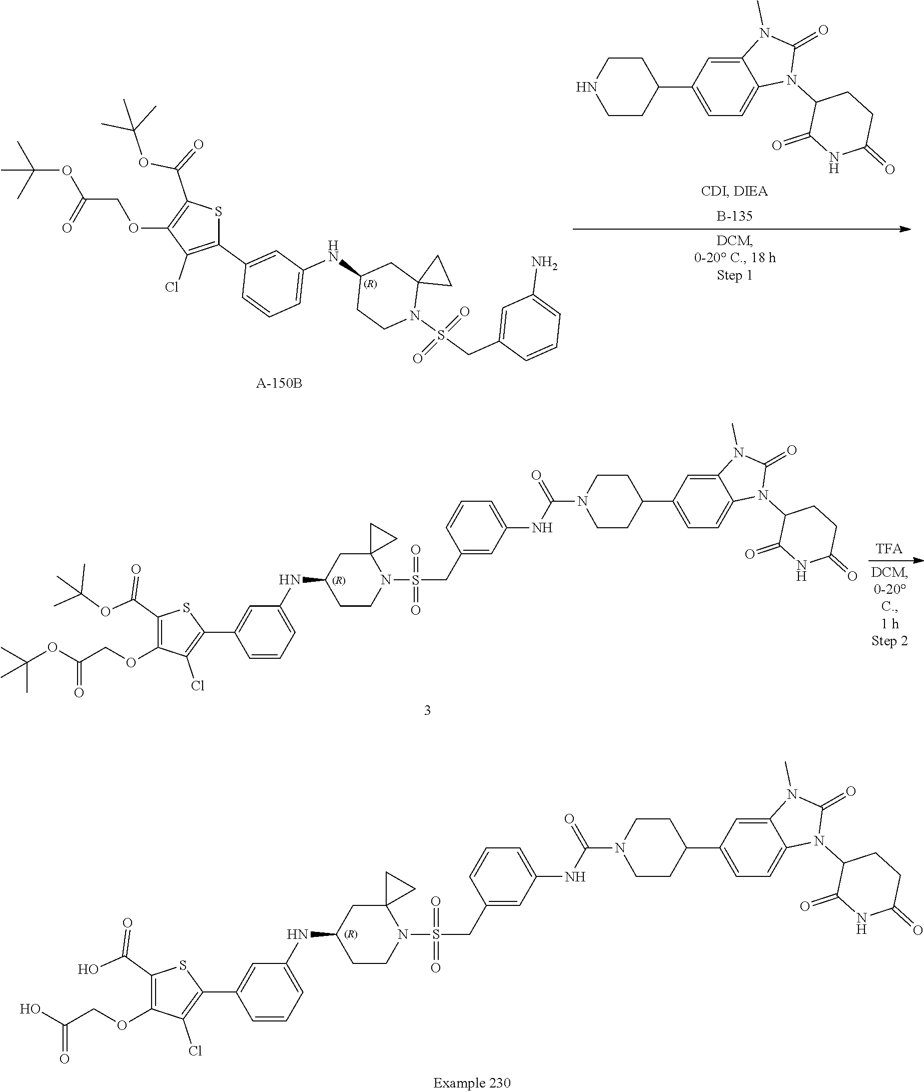

### Step 1: tert-butyl 3-(2-(tert-butoxy)-2-oxoethoxy)-4-chloro-5-(3-(((7R)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylate (3)

tert-butyl 3-(2-(tert-butoxy)-2-oxoethoxy)-4-chloro-5-(3-(((7R)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylate (3, 100 mg, 92.02 μmol, 66% yield) was synthesized from (R)-tert-butyl 5-(3-((4-((3-aminobenzyl)sulfonyl)-4-azaspiro[2.5]octan-7-yl)amino)phenyl)-3-(2-(tert-butoxy)-2-oxoethoxy)-4-chlorothiophene-2-carboxylate (A-150B, 100 mg, 139.21 μmol) and 3-(3-methyl-2-oxo-5-(piperidin-4-yl)-2,3-dihydro-1H-benzo[d]imidazol-1-yl)piperidine-2,6-dione (B-135, 47.67 mg, 139.21 μmol) in a similar fashion to Compound B-242. The residue was purified by column chromatography (SiO₂, Petroleum ether:Ethyl acetate=1:0 to 0:1).

LCMS (ESI): m/z 1086.8 [M+H]⁺

### Step 2: 3-(carboxymethoxy)-4-chloro-5-(3-(((7R)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2,5]octan-7-yl)amino)phenyl)thiophene-2-carboxylic acid (230)

3-(carboxymethoxy)-4-chloro-5-(3-(((7R)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylic acid (230, 50.91 mg, 45.37 μmol, 49% yield, TFA salt) was synthesized from tert-butyl 3-(2-(tert-butoxy)-2-oxoethoxy)-4-chloro-5-(3-(((7R)-4-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-4-azaspiro[2.5]octan-7-yl)amino)phenyl)thiophene-2-carboxylate (3, 100 mg, 92.02 μmol) in a similar fashion to Compound A-26. The residue was purified by prep-HPLC (flow: 25 mL/min; gradient: from 72%-42% water (0.1% TFA)-ACN; column: 3_Phenomenex Luna C18 150×25 mm×10 μm).

LCMS (ESI): m/z 974.2 [M+H]⁺

¹H NMR (400 MHz, DMSO-d₆) δ 11.08 (s, 1H), 8.60 (s, 1H), 7.48-7.44 (m, 2H), 7.22-7.16 (m, 2H), 7.12 (s, 1H), 7.04-7.00 (m, 1H), 6.96-6.92 (m, 1H), 6.87-6.83 (m, 2H), 6.80 (d, J=7.8 Hz, 1H), 6.68 (br d, J=8.2 Hz, 1H), 5.34 (dd, J=5.3, 12.8 Hz, 1H), 4.90 (s, 2H), 4.30 (br s, 3H), 4.16 (br s, 1H), 3.54 (br d, J=13.4 Hz, 1H), 3.34 (s, 3H), 3.02 (br t, J=12.1 Hz, 1H), 2.93-2.85 (m, 3H), 2.83-2.75 (m, 1H), 2.74-2.58 (m, 3H), 2.02-1.94 (m, 2H), 1.83-1.73 (m, 3H), 1.68-1.58 (m, 2H), 1.56-1.46 (m, 1H), 1.32 (br d, J=11.0 Hz, 1H), 0.66-0.60 (m, 2H), 0.54-0.46 (m, 2H).

### Example 231

### 3-(carboxymethoxy)-4-chloro-5-(3-(((7R)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylic acid (231)

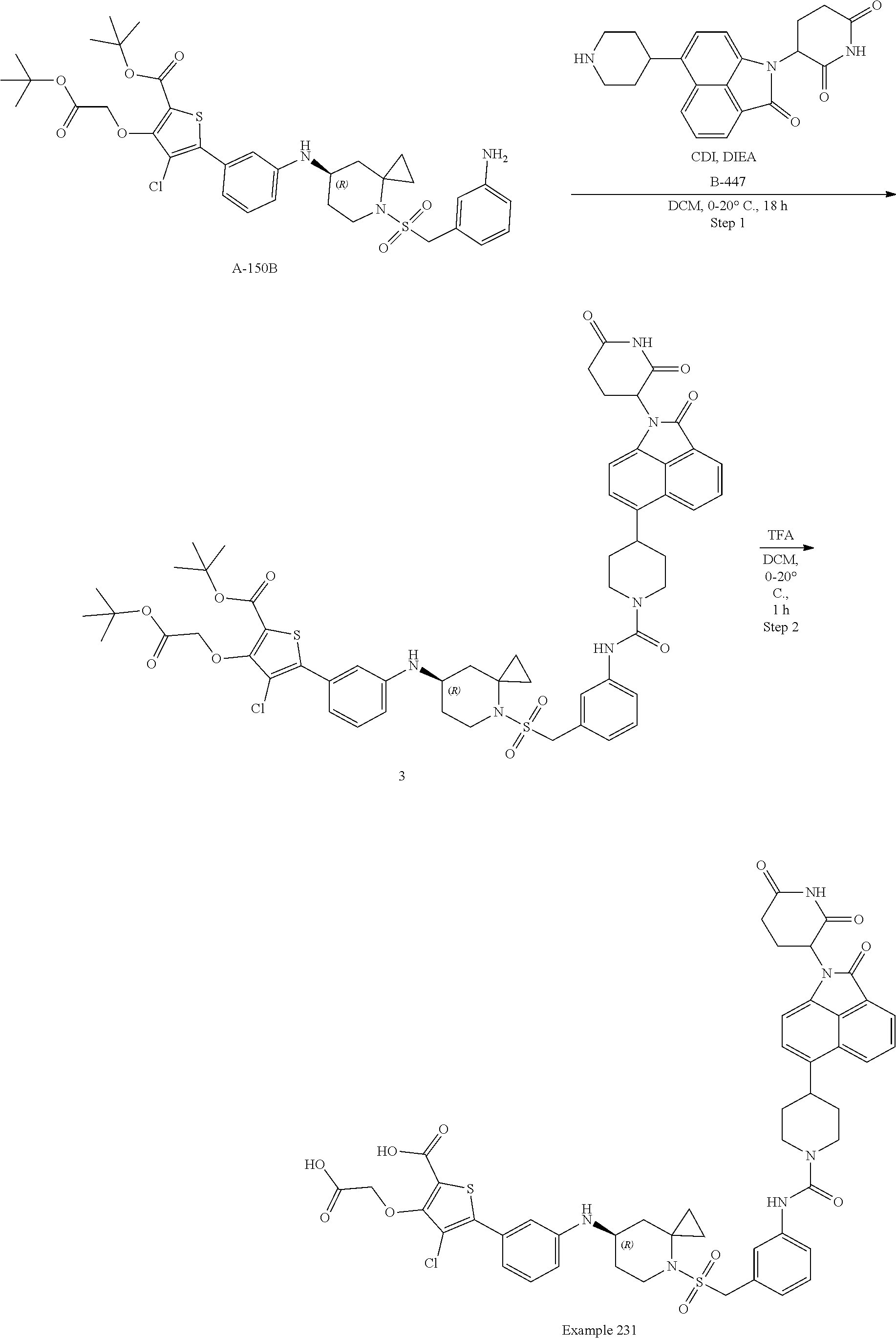

### Step 1: tert-butyl 3-(2-(tert-butoxy)-2-oxoethoxy)-4-chloro-5-(3-(((7R)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylate (3)

tert-butyl 3-(2-(tert-butoxy)-2-oxoethoxy)-4-chloro-5-(3-(((7R)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylate (3, 100 mg, 90.28 μmol, 65% yield) was synthesized from (R)-tert-butyl 5-(3-((4-((3-aminobenzyl)sulfonyl)-4-azaspiro[2.5]octan-7-yl)amino)phenyl)-3-(2-(tert-butoxy)-2-oxoethoxy)-4-chlorothiophene-2-carboxylate (A-150B, 100 mg, 139.21 μmol) and 3-(2-oxo-6-(piperidin-4-yl)benzo[cd]indol-[(2H)-yl)piperidine-2,6-dione (B-447, 55.67 mg, 139.21 μmol) in a similar fashion to Compound B-242. The residue was purified by column chromatography (SiO₂, Petroleum ether:Ethyl acetate=1:0 to 0:1).

LCMS (ESI): m/z 1107.6 [M+H]⁺

### Step 2: 3-(carboxymethoxy)-4-chloro-5-(3-(((7R)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylic acid (231)

3-(carboxymethoxy)-4-chloro-5-(3-(((7R)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylic acid (231, 39.89 mg, 35.59 μmol, 39% yield, TFA salt) was synthesized from tert-butyl 3-(2-(tert-butoxy)-2-oxoethoxy)-4-chloro-5-(3-(((7R)-4-((3-(4-(1-(2,6-dioxopiperidin-3-yl)-2-oxo-1,2-dihydrobenzo[cd]indol-6-yl)piperidine-1-carboxamido)benzyl)sulfonyl)-4-azaspiro[2.5]octan-7-yl)amino)phenyl)thiophene-2-carboxylate (3, 100 mg, 90.28 μmol) in a similar fashion to Compound A-26. The residue was purified by prep-HPLC (flow: 25 mL/min; gradient: from 72%-42% water (0.1% TFA)-ACN; column: 3_Phenomenex Luna C18 150×25 mm×10 μm). LCMS (ESI): m/z 999.5 [M+H]⁺

¹H NMR (400 MHz, DMSO-d₆) δ 11.10 (s, 1H), 8.64 (s, 1H), 8.52 (d, J=8.4 Hz, 1H), 8.12 (d, J=6.8 Hz, 1H), 7.93-7.85 (m, 1H), 7.50-7.44 (m, 2H), 7.40 (d, J=7.6 Hz, 1H), 7.20 (td, J=8.0, 16.0 Hz, 2H), 7.10 (d, J=7.4 Hz, 1H), 6.88-6.82 (m, 2H), 6.78 (br d, J=7.8 Hz, 1H), 6.68 (br d, J=7.8 Hz, 1H), 5.44 (br dd, J=5.1, 12.8 Hz, 1H), 4.90 (s, 2H), 4.32 (br d, J=14.2 Hz, 3H), 4.20 (s, 1H), 3.62-3.52 (m, 3H), 3.11-3.01 (m, 3H), 2.98-2.92 (m, 1H), 2.80-2.70 (m, 2H), 2.12-2.06 (m, 1H), 2.01-1.86 (m, 3H), 1.80-1.66 (m, 3H), 1.58-1.44 (m, 1H), 1.36-1.28 (m, 1H), 0.68-0.60 (m, 2H), 0.56-0.44 (m, 2H).

### Example 232

### 3-(carboxymethoxy)-4-chloro-5-[3-[[(4S)-1-[[3-[2-[1-[1-(2,6-dioxo-3-piperidyl)-3-methyl-indol-5-yl]-4-piperidyl]ethylcarbamoylamino]phenyl]methylsulfonyl]-2,2-dimethyl-4-piperidyl]amino]phenyl]thiophene-2-carboxylic acid (232)

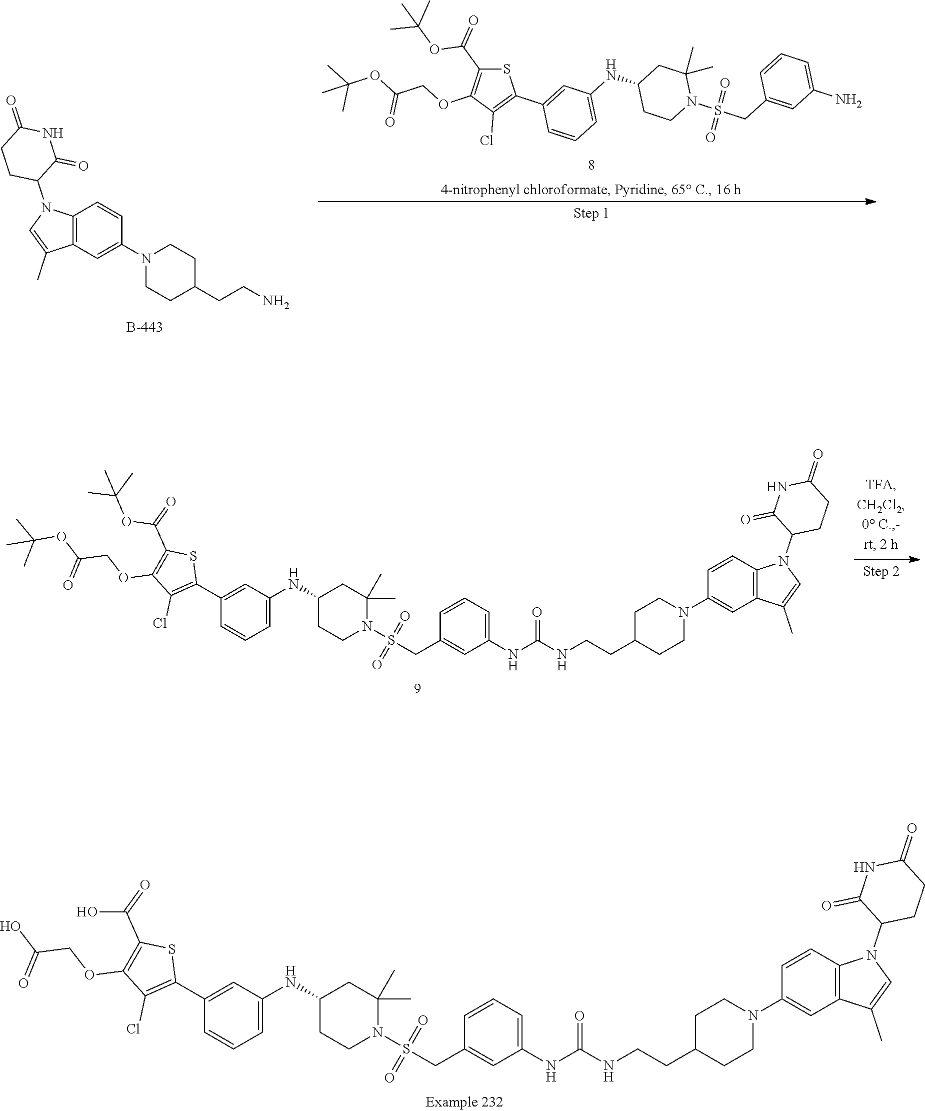 [SMILES: Cc1cn(C2CCC(=O)NC2=O)c2ccc(N3CCC(CCN)CC3)cc12 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cc1cn(C2CCC(=O)NC2=O)c2ccc(N3CCC(CCNC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)CC3)cc12 ; Cc1cn(C2CCC(=O)NC2=O)c2ccc(N3CCC(CCNC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc12]

### Step 1: tert-butyl 3-(2-tert-butoxy-2-oxo-ethoxy)-4-chloro-5-[3-[[(4S)-1-[[3-[2-[1-[1-(2,6-dioxo-3-piperidyl)-3-methyl-indol-5-yl]-4-piperidyl]ethylcarbamoylamino]phenyl]methylsulfonyl]-2,2-dimethyl-4-piperidyl]amino]phenyl]thiophene-2-carboxylate (9)

tert-butyl 3-(2-tert-butoxy-2-oxo-ethoxy)-4-chloro-5-[3-[[(4S)-1-[3-[2-[1-[1-(2,6-dioxo-3-piperidyl)-3-methyl-indol-5-yl]-4-piperidyl]ethylcarbamoylamino]phenyl]methylsulfonyl]-2,2-dimethyl-4-piperidyl]amino]phenyl]thiophene-2-carboxylate (9, 350 mg, 103.61 μmol, 50% yield, 33% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (8, 150 mg, 206.45 μmol) and 3-[5-[4-(2-aminoethyl)-1-piperidyl]-3-methyl-indol-1-yl]piperidine-2,6-dione (B-443, 136.45 mg, 206.45 μmol, TFA salt) in a similar fashion to Example 180, except with heating to 65 C. After concentration, the residue was triturated with cold water (20 mL). The solid was filtered, and the material was used in the next step without further purification. LCMS (ES+): m/z 1114.7 [M+H]⁺

### Step 2: 3-(carboxymethoxy)-4-chloro-5-[3-[[(4S)-1-[3-[2-[1-[1-(2,6-dioxo-3-piperidyl)-3-methyl-indol-5-yl]-4-piperidyl]ethylcarbamoylamino]phenyl]methylsulfonyl]-2,2-dimethyl-4-piperidyl]amino]phenyl]thiophene-2-carboxylic acid (232)

3-(carboxymethoxy)-4-chloro-5-[3-[[(4S)-1-[[3-[2-[1-[1-(2,6-dioxo-3-piperidyl)-3-methyl-indol-5-yl]-4-piperidyl]ethylcarbamoylamino]phenyl]methylsulfonyl]-2,2-dimethyl-4-piperidyl]amino]phenyl]thiophene-2-carboxylic acid (232, 25 mg, 20.74 μmol, 20% yield, TFA salt) was synthesized from tert-butyl 3-(2-tert-butoxy-2-oxo-ethoxy)-4-chloro-5-[3-[[(4S)-1-[[3-[2-[1-[1-(2,6-dioxo-3-piperidyl)-3-methyl-indol-5-yl]-4-piperidyl]ethylcarbamoylamino]phenyl]methylsulfonyl]-2,2-dimethyl-4-piperidyl]amino]phenyl]thiophene-2-carboxylate (9, 350 mg, 103.61 μmol) in a similar fashion to Compound A-26. The residue was purified by reverse phase prep HPLC [Purification method: XSelect C18 (150×19) mm, 5 microns; Mobile phase A: 0.1% TFA in water; Mobile phase B: MeCN). LCMS (ES+): m/z 1002.2 [M+H]⁺

¹HNMR (400 MHz, DMSO-d6): δ 13.30 (bs, 1H), 11.10 (s, 1H), 8.61 (s, 1H), 7.87 (s, 1H), 7.58 (d, J=8.80 Hz, 1H), 7.45-7.39 (m, 3H), 7.35 (s, 1H), 7.26-7.19 (m, 2H), 6.95 (dd, J=3.20, 10.60 Hz, 1H), 6.88 (s, 1H), 6.82 (d, J=7.20 Hz, 1H), 6.70 (d, J=8.40 Hz, 1H), 6.21 (t, J=5.60 Hz, 1H), 5.70-5.60 (m, 2H), 4.92 (s, 2H), 4.32 (dd, J=13.20, 45.60 Hz, 2H), 3.51-3.35 (m, 1H), 3.23-3.10 (m, 3H), 2.94-2.86 (m, 1H), 2.75-2.70 (m, 1H), 2.28 (s, 3H), 2.14-2.04 (m, 3H), 1.89-1.78 (m, 3H), 1.71-1.59 (m, 2H), 1.55-1.39 (m, 9H), 1.16-1.08 (m, 1H).

### Example 233

### 3-(carboxymethoxy)-4-chloro-5-[3-[[(4S)-1-[[3-[[4-[1-(2,6-dioxo-3-piperidyl)-3-methyl-indazol-5-yl]piperidine-1-carbonyl]amino]phenyl]methylsulfonyl]-2,2-dimethyl-4-piperidyl]amino]phenyl]thiophene-2-carboxylic acid (233)

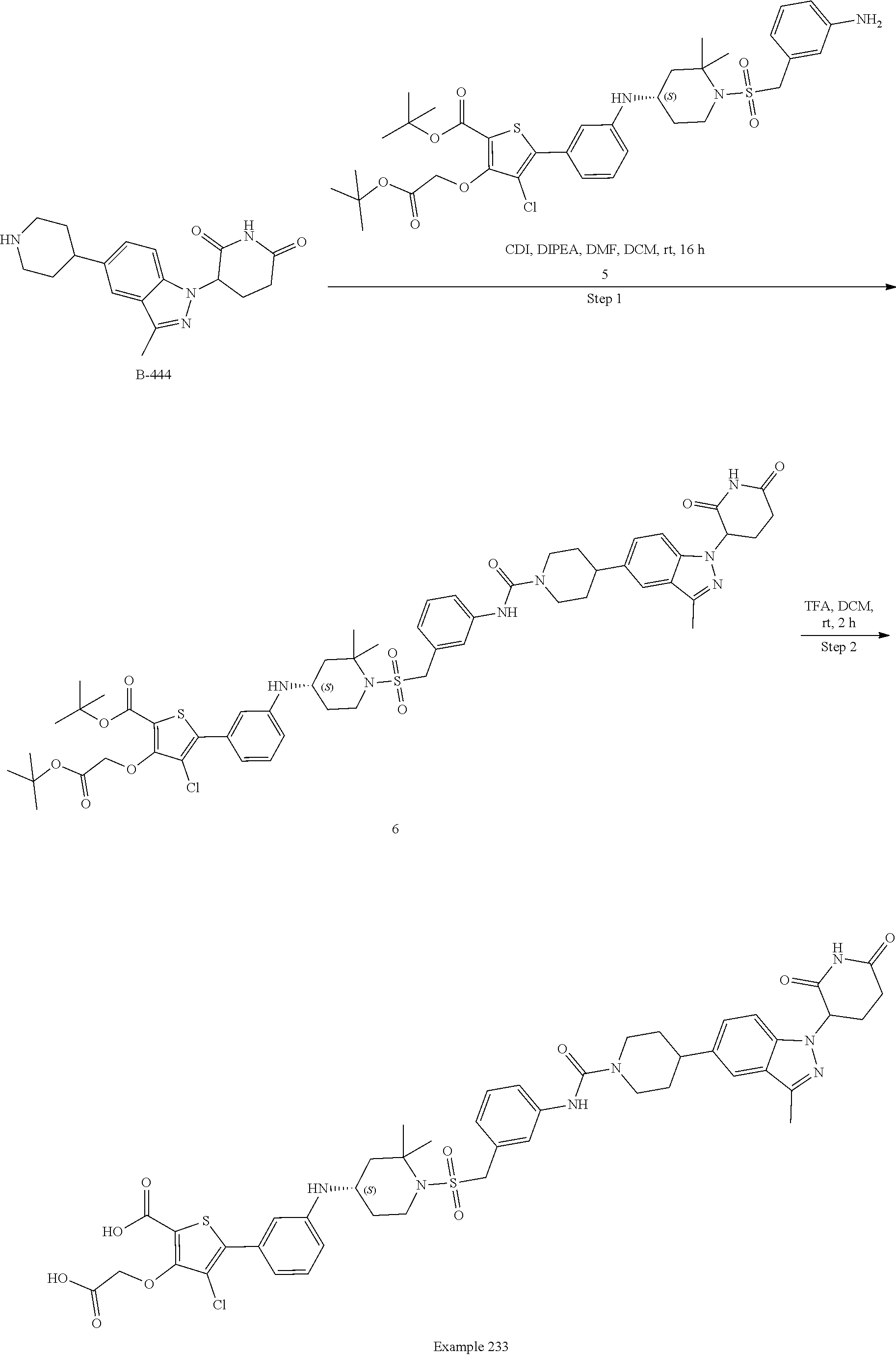

### Step 1: tert-butyl 3-(2-tert-butoxy-2-oxo-ethoxy)-4-chloro-5-[3-[[(4S)-1-[[3-[[4-[1-(2,6-dioxo-3-piperidyl)-3-methyl-indazol-5-yl]piperidine-1-carbonyl]amino]phenyl]methylsulfonyl]-2,2-dimethyl-4-piperidyl]amino]phenyl]thiophene-2-carboxylate (6)

tert-butyl 3-(2-tert-butoxy-2-oxo-ethoxy)-4-chloro-5-[3-[[(4S)-1-[[3-[[4-[1-(2,6-dioxo-3-piperidyl)-3-methyl-indazol-5-yl]piperidine-1-carbonyl]amino]phenyl]methylsulfonyl]-2,2-dimethyl-4-piperidyl]amino]phenyl]thiophene-2-carboxy late (6, 90 mg, 65.16 μmol, 47% yield, formate salt) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (5, 100 mg, 138.82 μmol) and 3-[3-methyl-5-(4-piperidyl) indazol-1-yl]piperidine-2,6-dione (B-444, 81.52 mg, 138.82 μmol, TFA salt) in a similar fashion to Compound B-242. The residue was purified by reverse phase column chromatography [Silicycle C18 column; Mobile phase A: 0.1% formic acid in water; Mobile phase B: MeCN].

LCMS (ES−): m/z 1070.2 [M−H]⁻

### Step 2: 3-(carboxymethoxy)-4-chloro-5-[3-[[(4S)-1-[[3-[[4-[1-(2,6-dioxo-3-piperidyl)-3-methyl-indazol-5-yl]piperidine-1-carbonyl]amino]phenyl]methylsulfonyl]-2,2-dimethyl-4-piperidyl]amino]phenyl]thiophene-2-carboxylic acid (233)

3-(carboxymethoxy)-4-chloro-5-[3-[[(4S)-1-[[3-[[4-[1-(2,6-dioxo-3-piperidyl)-3-methyl-indazol-5-yl]piperidine-1-carbonyl]amino]phenyl]methylsulfonyl]-2,2-dimethyl-4-piperidyl]amino]phenyl]thiophene-2-carboxylic acid (233, 35 mg, 33.75 μmol, 50% yield, formic acid salt) was synthesized from tert-butyl 3-(2-tert-butoxy-2-oxo-ethoxy)-4-chloro-5-[3-[[(4S)-1-[[3-[[4-[1-(2,6-dioxo-3-piperidyl)-3-methyl-indazol-5-yl]piperidine-1-carbonyl]amino]phenyl]methylsulfonyl]-2,2-dimethyl-4-piperidyl]amino]phenyl]thiophene-2-carboxylate (6, 90 mg, 67.96 μmol, TFA salt) in a similar fashion to Compound A-26. The residue was purified by reverse phase column chromatography [Silicycle C18 column; Mobile phase A: 0.1% formic acid in water; Mobile phase B: MeCN]. LCMS (ES−): m/z 960.2 [M−H]⁺

¹HNMR (400 MHz, DMSO-d6): δ 11.05 (s, 1H), 8.63 (s, 1H), 7.59-7.57 (m, 2H), 7.50-7.46 (m, 2H), 7.33 (dd, J=1.20, 8.80 Hz, 1H), 7.25 (t, J=8.00 Hz, 1H), 7.19 (t, J=8.00 Hz, 1H), 6.99 (d, J=7.60 Hz, 1H), 6.85 (s, 1H), 6.79 (d, J=7.60 Hz, 1H), 6.68 (dd, J=1.20, 8.20 Hz, 1H), 5.73-5.65 (m, 2H), 4.79 (s, 2H), 4.39-4.24 (m, 4H), 3.55-3.33 (m, 2H), 3.16-3.13 (m, 2H), 2.95-2.83 (m, 4H), 2.73-2.69 (m, 1H), 2.46 (s, 3H), 2.28-2.18 (m, 1H), 1.87-1.77 (m, 4H), 1.68-1.50 (m, 2H), 1.49-1.44 (m, 8H), 1.15-1.05 (m, 1H).

### Example 234 and Example 235

### 3-(carboxymethoxy)-4-chloro-5-[3-[[(4S)-1-[[3-[[4-[1-[(3S)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (234) and 3-(carboxymethoxy)-4-chloro-5-[3-[[(4S)-1-[3-[4-[1-[(3R)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (235)

Configurations are arbitrarily assigned.

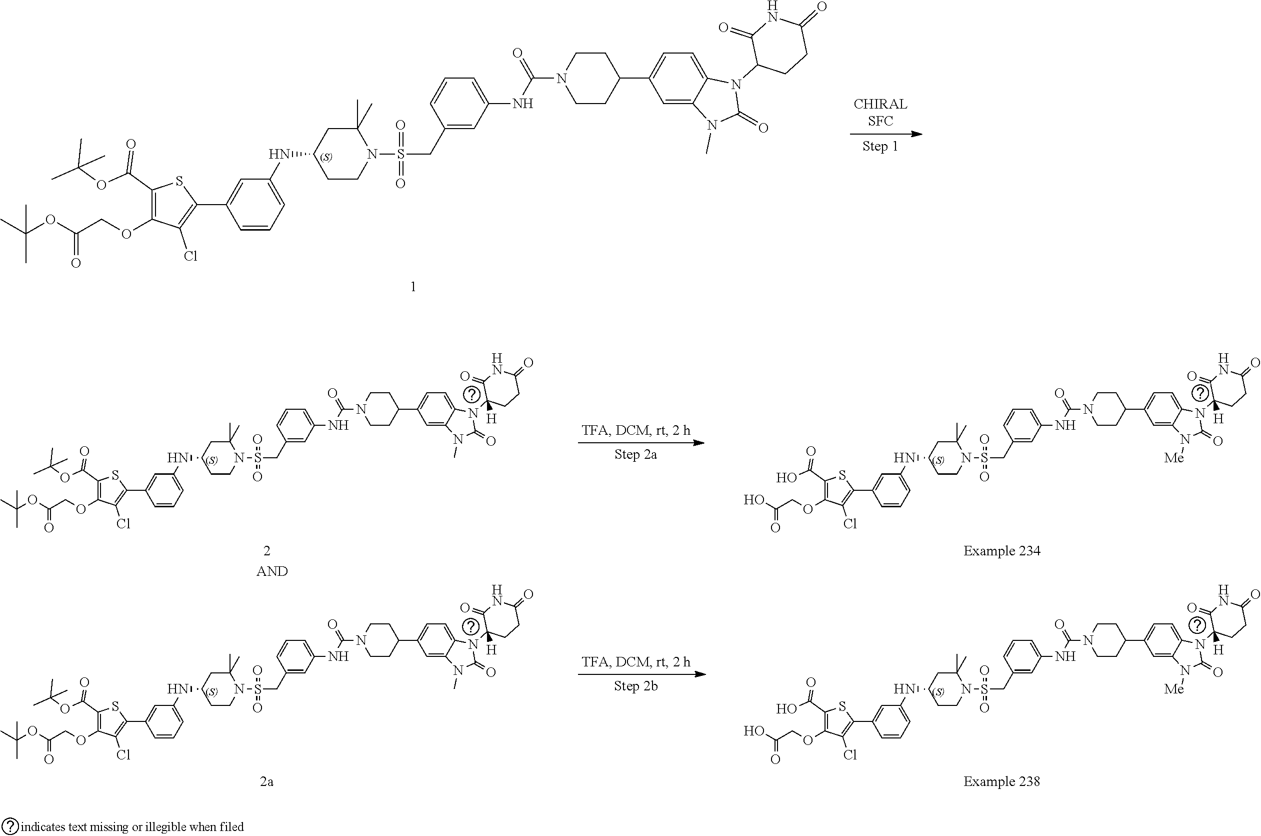

350 mg of tert-butyl 3-(2-(tert-butoxy)-2-oxoethoxy)-4-chloro-5-(3-(((4S)-1-((3-(4-(1-(2,6-dioxopiperidin-3-yl)-3-methyl-2-oxo-2,3-dihydro-1H-benzo[d]imidazol-5-yl)piperidine-1-carboxamido)benzyl)sulfonyl)-2,2-dimethylpiperidin-4-yl)amino)phenyl)thiophene-2-carboxylate (1) is dissolved in 5 mL of Acetonitrile and injected 0.5 mL per injection.

### Chiral SFC:

Instrument: PIC 175
Column: (R,R) Whelk-01 (250*30) mm, 5p
Mobile Phase: {(CO₂: 0.2% TFA in IPA:MeCN (1:1) (60:40)}
Total flow: 100 g/min
Back pressure: 100 bar
Wavelength: 210 nm
Cycle time: 11.50 min

After chiral separation both the fractions were collected and concentrated at 35° C. under reduced pressure to obtain 2 and 2a. The LCMS analysis indicated the transesterification side product (isopropyl ester) along with desired compound.

Fraction 1-Diastereomer 1 (2a): tert-butyl 3-(2-tert-butoxy-2-oxo-ethoxy)-4-chloro-5-[3-[[(4S)-1-[[3-[[4-[1-[(3R)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (2a, 130 mg, 103.41 μmol, 15% yield).

LCMS (ES+): m/z 1088.0 [M+H]⁺

Fraction 2-Diastereomer 2 (2): tert-butyl 3-(2-tert-butoxy-2-oxo-ethoxy)-4-chloro-5-[3-[[(4S)-1-[[3-[4-[1-[(3S)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (2, 120 mg, 96.55 μmol, 14% yield).

LCMS (ES+): m/z 1088.0 [M+H]⁺

### Step 2: 3-(carboxymethoxy)-4-chloro-5-[3-[[(4S)-1-[[3-[[4-[1-[(3S)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (234)

3-(carboxymethoxy)-4-chloro-5-[3-[[(4S)-1-[[3-[[4-[1-[(3S)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (234, 83 mg, 75.52 μmol, 80% yield, TFA salt) was synthesized from tert-butyl 3-(2-tert-butoxy-2-oxo-ethoxy)-4-chloro-5-[3-[[(4S)-1-[3-[4-[1-[(3S)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (2, 120 mg, 94.79 μmol) in a similar fashion to Compound A-26. The residue was purified by reverse phase prep HPLC [Purification method: Column: Sunfire C18 5 micron, (19×150) mm; Mobile phase A: 0.1% TFA in water; Mobile phase B: Acetonitrile].

LCMS (ES+): m/z 976.0 [M+H]⁺

¹HNMR (400 MHz, DMSO-d6): δ 11.10 (s, 1H), 8.63 (s, 1H), 7.58 (s, 1H), 7.47 (d, J=8.40 Hz, 1H), 7.27-7.20 (m, 2H), 7.12 (s, 1H), 7.03-6.98 (m, 2H), 6.94-6.90 (m, 2H), 6.85 (d, J=7.60 Hz, 1H), 6.72 (d, J=8.00 Hz, 1H), 5.36-5.32 (m, 1H), 4.91 (s, 2H), 4.38-4.24 (m, 4H), 3.59-3.45 (m, 4H), 3.33 (s, 3H), 3.16-3.09 (m, 1H), 2.92-2.86 (m, 3H), 2.78-2.60 (m, 3H), 2.02-1.99 (m, 1H), 1.86-1.79 (m, 4H), 1.68-1.62 (m, 2H), 1.49-1.39 (m, 7H), 1.15-1.05 (m, 1H).

### Step 2b: 3-(carboxymethoxy)-4-chloro-5-[3-[[(4S)-1-[[3-[[4-[1-[(3R)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (235)

3-(carboxymethoxy)-4-chloro-5-[3-[[(4S)-1-[[3-[[4-[[1-[(3R)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylic acid (235, 77 mg, 69.75 μmol, 67% yield, TFA salt) was synthesized from tert-butyl 3-(2-tert-butoxy-2-oxo-ethoxy)-4-chloro-5-[3-[[(4S)-1-[[3-[[4-[1-[(3R)-2,6-dioxo-3-piperidyl]-3-methyl-2-oxo-benzimidazol-5-yl]piperidine-1-carbonyl]amino]phenyl]methylsulfonyl]-2,2-dimethyl-4-piperidyl]amino]phenyl]thiophene-2-carboxylate (2a, 130 mg, 103.51 μmol) in a similar fashion to Compound A-26. The residue was purified by reverse phase prep HPLC [Purification method: Column: Sunfire C18 5 micron, (19×150) mm; Mobile phase A: 0.1% TFA in water; Mobile phase B: Acetonitrile].

LCMS (ES+): m/z 976.0 [M+H]⁺

¹HNMR (400 MHz, DMSO-d₆): δ 11.10 (s, 1H), 8.63 (s, 1H), 7.58 (s, 1H), 7.47 (d, J=8.00 Hz, 1H), 7.27-7.20 (m, 2H), 7.12 (s, 1H), 7.03-6.98 (m, 2H), 6.94-6.90 (m, 2H), 6.85 (d, J=7.60 Hz, 1H), 6.72 (d, J=8.00 Hz, 1H), 5.36-5.32 (m, 1H), 4.91 (s, 2H), 4.38-4.24 (m, 4H), 3.58-3.45 (m, 4H), 3.33 (s, 3H), 3.16-3.10 (m, 1H), 2.92-2.81 (m, 3H), 2.78-2.65 (m, 3H), 2.02-1.99 (m, 1H), 1.87-1.79 (m, 4H), 1.68-1.59 (m, 2H), 1.49-1.39 (m, 7H), 1.15-1.05 (m, 1H).

### Example 236

### 3-(carboxymethoxy)-4-chloro-5-[3-[[(4S)-1-[[3-[[2-[4-[[1-(2,6-dioxo-3-piperidyl)-3-methyl-2-oxo-benzimidazol-5-yl]methyl]phenyl]acetyl]amino]phenyl]methylsulfonyl]-2,2-dimethyl-4-piperidyl]amino]phenyl]thiophene-2-carboxylic acid (236)

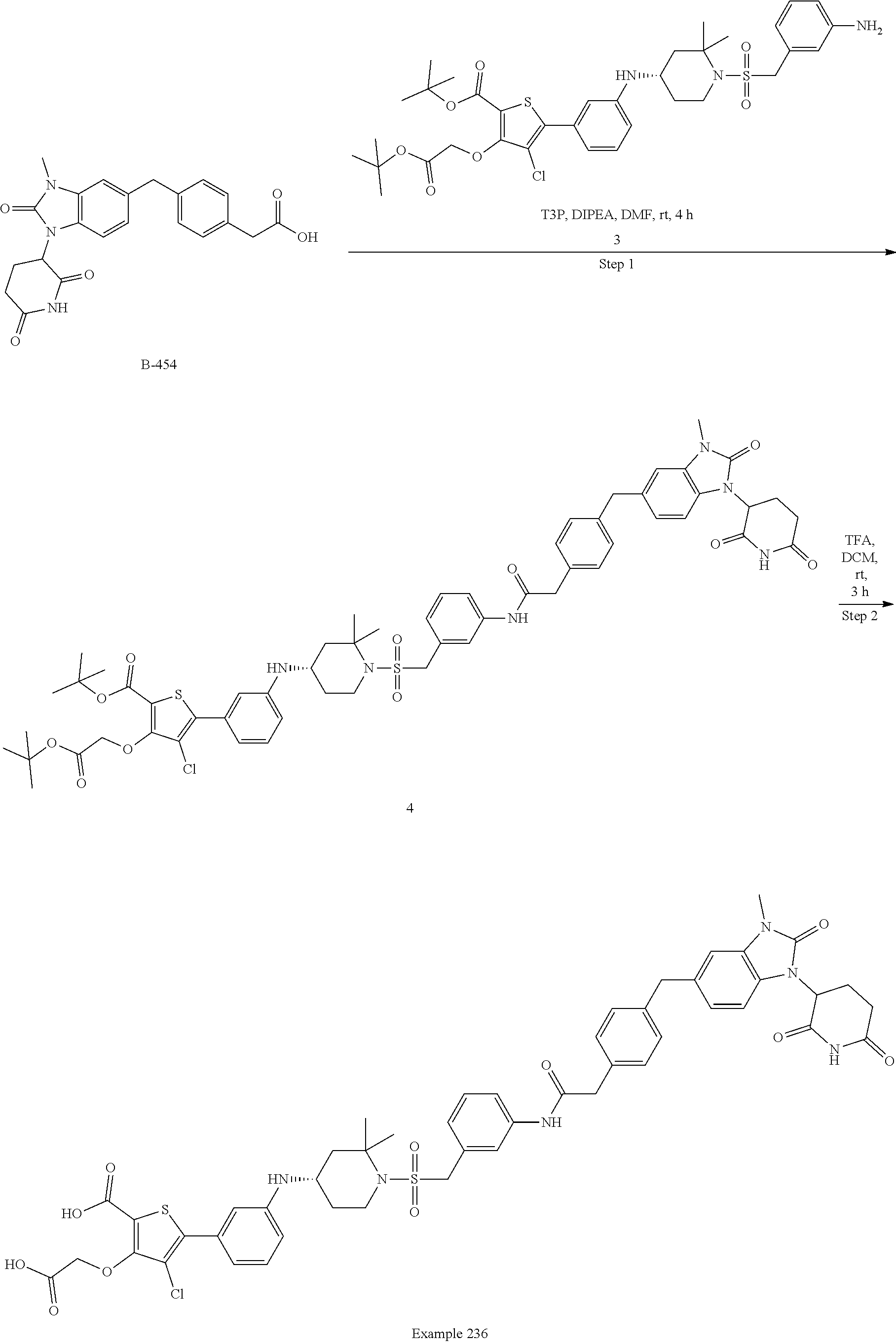 [SMILES: Cn1c(=O)n(C2CCC(=O)NC2=O)c2ccc(Cc3ccc(CC(=O)O)cc3)cc21 ; CC(C)(C)OC(=O)COc1c(C(=O)OC(C)(C)C)sc(-c2cccc(N[C@H]3CCN(S(=O)(=O)Cc4cccc(N)c4)C(C)(C)C3)c2)c1Cl ; Cn1c(=O)n(C2CCC(=O)NC2=O)c2ccc(Cc3ccc(CC(=O)Nc4cccc(CS(=O)(=O)N5CC[C@H](Nc6cccc(-c7sc(C(=O)OC(C)(C)C)c(OCC(=O)OC(C)(C)C)c7Cl)c6)CC5(C)C)c4)cc3)cc21 ; Cn1c(=O)n(C2CCC(=O)NC2=O)c2ccc(Cc3ccc(CC(=O)Nc4cccc(CS(=O)(=O)N5CC[C@H](Nc6cccc(-c7sc(C(=O)O)c(OCC(=O)O)c7Cl)c6)CC5(C)C)c4)cc3)cc21]

### Step 1: tert-butyl 3-(2-tert-butoxy-2-oxo-ethoxy)-4-chloro-5-[3-[[(4S)-1-[[3-[2-[4-[[1-(2,6-dioxo-3-piperidyl)-3-methyl-2-oxo-benzimidazol-5-yl]methyl]phenyl]acetyl]amino]phenyl]methylsulfonyl]-2,2-dimethyl-4-piperidyl]amino]phenyl]thiophene-2-carboxylate (4)

tert-butyl 3-(2-tert-butoxy-2-oxo-ethoxy)-4-chloro-5-[3-[[(4S)-1-[3-[2-[4-[[1-(2,6-dioxo-3-piperidyl)-3-methyl-2-oxo-benzimidazol-5-yl]methyl]phenyl]acetyl]amino]phenyl]methylsulfonyl]-2,2-dimethyl-4-piperidyl]amino]phenyl]thiophene-2-carboxylate (4, 200 mg, 138.77 μmol, 91% yield, 77% purity) was synthesized from tert-butyl 5-[3-[[(4S)-1-[(3-aminophenyl)methylsulfonyl]-2,2-dimethyl-4-piperidyl]amino]phenyl]-3-(2-tert-butoxy-2-oxo-ethoxy)-4-chloro-thiophene-2-carboxylate (3, 110 mg, 152.71 μmol) and 2-[4-[1-(2,6-dioxo-3-piperidyl)-3-methyl-2-oxo-benzimidazol-5-yl]methyl]phenyl]acetic acid (B-454, 62.84 mg, 152.71 μmol) in a similar fashion to Compound A-88, the reaction mixture was concentrated under vacuum and the residue was suspended in water. The solid was filtered and dried under vacuum.

LCMS (ES+): m/z 1111.0 [M+H]⁺

### Step 2: 3-(carboxymethoxy)-4-chloro-5-[3-[[(4S)-1-[[3-[[2-[4-[[1-(2,6-dioxo-3-piperidyl)-3-methyl-2-oxo-benzimidazol-5-yl]methyl]phenyl]acetyl]amino]phenyl]methylsulfonyl]-2,2-dimethyl-4-piperidyl]amino]phenyl]thiophene-2-carboxylic acid (236)

3-(carboxymethoxy)-4-chloro-5-[3-[[(4S)-1-[[3-[[2-[4-[1-(2,6-dioxo-3-piperidyl)-3-methyl-2-oxo-benzimidazol-5-yl]methyl]phenyl]acetyl]amino]phenyl]methylsulfonyl]-2,2-dimethyl-4-piperidyl]amino]phenyl]thiophene-2-carboxylic acid (236, 82 mg, 73.04 μmol, 53% yield, TFA salt) was synthesized from tert-butyl 3-(2-tert-butoxy-2-oxo-ethoxy)-4-chloro-5-[3-[[(4S)-1-[[3-[[2-[4-[[1-(2,6-dioxo-3-piperidyl)-3-methyl-2-oxo-benzimidazol-5-yl]methyl]phenyl]acetyl]amino]phenyl]methylsulfonyl]-2,2-dimethyl-4-piperidyl]amino]phenyl]thiophene-2-carboxylate (4, 200 mg, 138.77 μmol) in a similar fashion to Compound A-26. The residue was purified by reverse phase prep HPLC [Purification method: Column: XSelect C18 (150×19) mm, 5 micron; Mobile phase A: 0.1% TFA in water; Mobile phase B: MeCN].

LCMS (ES+): m/z 997.2 [M+H]⁺

¹HNMR (400 MHz, DMSO-d6): δ 13.20 (brs, 1H), 11.11 (s, 1H), 10.21 (s, 1H), 7.65 (s, 1H), 7.60 (d, J=8.40 Hz, 1H), 7.30 (t, J=8.00 Hz, 1H), 7.25-7.16 (m, 5H), 7.09-7.06 (m, 2H), 7.00 (d, J=8.00 Hz, 1H), 6.90-6.88 (m, 2H), 6.82 (d, J=8.00 Hz, 1H), 6.68 (dd, J=1.20, 8.20 Hz, 1H), 5.34-5.31 (m, 1H), 4.92 (s, 2H), 4.34 (dd, J=14.00, 46.80 Hz, 2H), 3.92 (s, 2H), 3.60 (s, 2H), 3.52-3.41 (m, 2H), 3.29 (s, 3H), 3.12-3.05 (m, 1H), 2.91-2.83 (m, 1H), 2.69-2.63 (m, 2H), 1.99-1.96 (m, 1H), 1.87-1.85 (m, 1H), 1.79-1.76 (m, 1H), 1.45-1.38 (m, 7H).

### Example 237 and Example 238

### 3-(carboxymethoxy)-4-chloro-5-[3-[[(4S)-1-[3-[2-[2-[1-[1-(2,6-dioxo-3-piperidyl)-3-methyl-2-oxo-benzimidazol-5-yl]-4-piperidyl]ethylamino]ethylamino]phenyl]methylsulfonyl]-2,2-dimethyl-4-piperidyl]amino]phenyl]thiophene-2-carboxylic acid (237) and 3-(carboxymethoxy)-4-chloro-5-[3-[(4S)-1-[3-[3-[2-[1-[1-(2,6-dioxo-3-piperidyl)-3-methyl-2-oxo-benzimidazol-5-yl]-4-piperidyl]ethyl]-2-oxo-imidazolidin-1-yl]phenyl]methylsulfonyl]-2,2-dimethyl-4-piperidyl]amino]phenyl]thiophene-2-carboxylic acid (238)

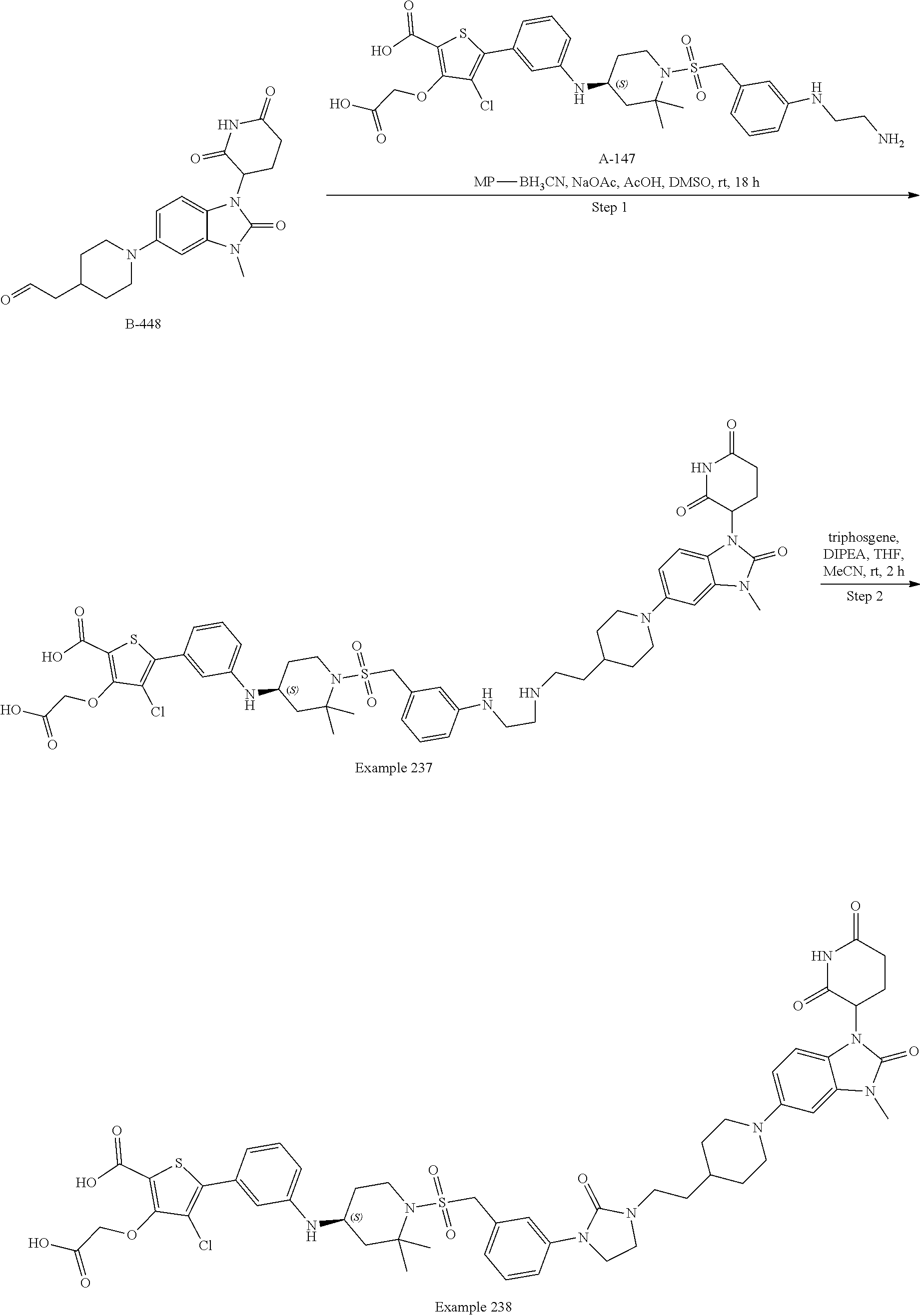

### Step 1: 3-(carboxymethoxy)-4-chloro-5-[3-[[(4S)-1-[3-[2-[2-[1-[1-(2,6-dioxo-3-piperidyl)-3-methyl-2-oxo-benzimidazol-5-yl]-4-piperidyl]ethylamino]ethylamino]phenyl]methylsulfonyl]-2,2-dimethyl-4-piperidyl]amino]phenyl]thiophene-2-carboxylic acid (237)

3-(carboxymethoxy)-4-chloro-5-[3-[[(4S)-1-[[3-[2-[2-[1-[1-(2,6-dioxo-3-piperidyl)-3-methyl-2-oxo-benzimidazol-5-yl]-4-piperidyl]ethylamino]ethylamino]phenyl]methylsulfonyl]-2,2-dimethyl-4-piperidyl]amino]phenyl]thiophene-2-carboxylic acid (237, 210 mg, 183.74 μmol, 32% yield, TFA salt) was synthesized from 5-[3-[[(4S)-1-[[3-(2-aminoethylamino)phenyl]methylsulfonyl]-2,2-dimethyl-4-piperidyl]amino]phenyl]-3-(carboxymethoxy)-4-chloro-thiophene-2-carboxylic acid (A-147, 0.4 g, 573.71 μmol, formic acid salt) and 2-[1-[1-(2,6-dioxo-3-piperidyl)-3-methyl-2-oxo-benzimidazol-5-yl]-4-piperidyl]acetaldehyde (B-446, 381.29 mg, 573.71 μmol, TFA salt) in a similar fashion to Compound A-74a/b, using DMSO/EtOH as solvent and including excess sodium acetate. The material was purified by reverse phase prep HPLC [Purification method: XSelect C18 (250×19) mm, 5 microns; Mobile phase A: 0.1% TFA in water; Mobile phase B: MeCN].

LCMS (ES+): m/z 1019.2 [M+H]⁺

### Step 2: 3-(carboxymethoxy)-4-chloro-5-[3-[[(4S)-1-[[3-[3-[2-[1-[1-(2,6-dioxo-3-piperidyl)-3-methyl-2-oxo-benzimidazol-5-yl]-4-piperidyl]ethyl]-2-oxo-imidazolidin-1-yl]phenyl]methylsulfonyl]-2,2-dimethyl-4-piperidyl]amino]phenyl]thiophene-2-carboxylic acid (238)

3-(carboxymethoxy)-4-chloro-5-[3-[[(4S)-1-[[3-[3-[2-[1-[1-(2,6-dioxo-3-piperidyl)-3-methyl-2-oxo-benzimidazol-5-yl]-4-piperidyl]ethyl]-2-oxo-imidazolidin-1-yl]phenyl]methylsulfonyl]-2,2-dimethyl-4-piperidyl]amino]phenyl]thiophene-2-carboxylic acid (238, 20 mg, 16.54 μmol, 13% yield, TFA salt) was synthesized from 3-(carboxymethoxy)-4-chloro-5-[3-[[(4S)-1-[[3-[2-[2-[1-[1-(2,6-dioxo-3-piperidyl)-3-methyl-2-oxo-benzimidazol-5-yl]-4-piperidyl]ethylamino]ethylamino]phenyl]methylsulfonyl]-2,2-dimethyl-4-piperidyl]amino]phenyl]thiophene-2-carboxylic acid (237, 150 mg, 131.24 μmol, TFA salt) in a similar fashion to Example 142, using THE/Acetonitrile as the solvent. The residue was subjected to reverse phase prep HPLC [Purification method: XSelect C18 (150×19) mm, 5 microns column; Mobile phase A: 0.1% TFA in water; Mobile phase B: MeCN]. LCMS (ES+): m/z 1045.2 [M+H]⁺

¹HNMR (400 MHz, DMSO-d6): δ 13.20 (brs, 1H), 11.11 (s, 1H), 7.64 (s, 1H), 7.59 (d, J=8.40 Hz, 1H), 7.36-7.32 (m, 2H), 7.23-7.19 (m, 2H), 7.07-7.05 (m, 2H), 6.87 (s, 1H), 6.81 (d, J=7.60 Hz, 1H), 6.70 (d, J=8.00 Hz, 1H), 5.45-5.35 (m, 1H), 4.92 (s, 2H), 4.36 (dd, J=13.60, 45.40 Hz, 2H), 3.85-3.81 (m, 3H), 3.44-3.29 (m, 6H), 3.15-3.11 (m, 2H), 2.91-2.82 (m, 2H), 2.78-2.61 (m, 3H), 2.09-2.00 (m, 3H), 1.91-1.78 (m, 2H), 1.66-1.51 (m, 4H), 1.46-1.36 (m, 8H), 1.24-1.11 (m, 1H).

### HiBIT Method

### Materials

Dulbecco's modified Eagle medium (DMEM) without phenol red and fetal bovine serum (FBS) were purchased from Gibco (Grand Island, NY, USA). Nano-Glo® HiBIT Lytic Assay System was purchased from Promega (Madison, WI, USA). 293T.109 (HiBIT-PTPN2) cell line was generated by ectopically expressing PTPN2 with N-terminal HiBIT fusion tag in 293T WT cell line purchased from ATCC (Manassas, VA, USA). Cell culture flasks and 384-well microplates were acquired from VWR (Radnor, PA, USA).

### PTPN2 Degradation Analysis

PTPN2 degradation was determined based on quantification of luminescent signal using Nano-Glo® HiBIT Lytic Assay kit. Test compounds were added to the 384-well plate from a top concentration of 10 μM with 11 points, half log titration in duplicates. 293T.109 cells were added into 384-well plates at a cell density of 5000 cells per well. The plates were kept at 37° C. with 5% CO₂ for 24 hours. The cells treated in the absence of the test compound were the negative control and the cells without Nano-Glo® HiBIT Lytic reagent were the positive control. After 24-hour incubation, Nano-Glo® HiBiT Lytic Assay reagents were added to the cells. Luminescence was acquired on EnVision™ Multilabel Reader (PerkinElmer, Santa Clara, CA, USA).

TABLE 2

| | |
|---|---|
| HiBiT Degradation | HiBiT Degradation |
| 293T.109 PTPN2 | B16F10.4 PTPN2 |
| 24.0 hours | 24.0 hours |

| Cmpd. | DC₅₀ | | DC₅₀ | |
|---|---|---|---|---|
| No. | (nM) | Eₘₐₓ | (nM) | Eₘₐₓ |
| 1 | C | C | D | C |
| 2 | C | B | D | C |
| 3 | C | B | D | B |
| 4 | C | B | D | B |
| 5 | C | B | N/A | N/A |
| 6 | C | B | N/A | N/A |
| 7 | C | B | D | B |
| 8 | C | B | C | B |
| 9 | C | B | C | B |
| 10 | C | B | C | A |
| 11 | C | B | C | B |
| 12 | C | B | C | B |
| 13 | C | B | C | B |
| 14 | C | B | D | B |
| 15 | C | B | C | B |
| 16 | C | B | D | B |
| 17 | D | D | D | C |
| 18 | C | B | C | B |
| 19 | C | B | C | A |
| 20 | C | B | D | B |
| 21 | C | B | D | C |
| 22 | C | B | D | C |
| 23 | C | B | C | B |
| 24 | C | B | D | B |
| 25 | C | A | C | A |
| 26 | C | B | D | C |
| 27 | C | B | D | B |
| 28 | C | B | D | B |
| 29 | C | B | C | A |
| 30 | C | B | D | C |
| 31 | C | A | D | D |
| 32 | B | A | C | B |
| 33 | C | A | D | C |
| 34 | C | A | C | B |
| 35 | C | A | C | B |
| 36 | C | B | C | B |
| 37 | C | C | C | B |
| 38 | C | B | D | C |
| 39 | C | B | D | C |
| 40 | C | B | D | C |
| 41 | B | B | D | C |
| 42 | C | B | D | C |
| 43 | B | A | D | C |
| 44 | A | A | C | B |
| 44 | A | A | C | B |
| 44a | A | A | C | A |
| 44b | A | A | C | A |
| 45 | C | B | C | B |
| 46 | A | A | C | B |
| 47 | C | B | C | A |
| 48 | A | A | C | A |
| 49 | C | A | C | B |
| 50 | A | A | D | C |
| 51 | B | A | B | A |
| 52 | C | B | C | B |
| 53 | B | A | C | A |
| 54 | D | B | D | C |
| 55 | B | A | B | A |
| 56 | D | D | D | D |
| 57 | B | B | D | C |
| 58 | C | B | D | C |
| 59 | C | B | C | A |
| 60 | B | A | A | A |
| 61 | C | A | C | B |
| 62 | B | A | C | B |
| 63 | B | A | C | A |
| 64 | C | A | C | B |
| 65 | C | A | C | A |
| 66 | C | B | D | C |
| 67 | C | B | D | C |
| 68 | B | A | B | A |
| 69 | A | A | B | A |
| 70 | B | A | C | B |
| 71 | B | A | D | C |
| 72 | B | A | B | A |
| 73 | B | A | D | B |
| 74 | A | A | D | A |
| 75 | A | A | D | B |
| 76 | C | A | D | A |
| 77 | B | A | D | B |
| 78 | A | A | D | B |
| 79 | A | A | B | A |
| 80 | B | A | B | A |
| 81 | B | A | B | A |
| 82 | A | A | A | A |
| 83 | A | A | B | A |
| 84 | C | B | D | C |
| 85 | B | A | C | B |
| 86 | B | A | C | B |
| 87 | B | A | B | A |
| 87 | B | A | A | A |
| 87a | B | A | B | A |
| 87b | B | A | B | A |
| 88 | A | A | B | A |
| 89 | C | A | D | B |
| 90 | A | A | C | B |
| 91 | B | A | B | A |
| 92 | B | A | C | B |
| 93 | B | A | C | B |
| 94 | C | B | C | C |
| 95 | C | B | C | B |
| 96 | B | C | C | C |
| 97 | C | B | D | C |
| 98 | B | B | C | A |
| 99 | B | A | A | A |
| 100 | C | B | C | B |
| 101 | B | A | B | A |
| 102 | B | A | B | A |
| 103 | C | B | B | A |
| 104 | B | A | A | A |
| 105 | B | A | C | A |
| 106 | B | A | B | A |
| 107 | B | B | C | B |
| 108 | B | B | D | C |
| 109 | A | A | C | B |
| 110 | B | B | C | B |
| 111 | C | C | D | C |
| 112 | B | A | B | A |
| 113 | B | A | B | A |
| 114 | B | A | A | A |
| 115 | B | A | C | A |
| 116 | B | A | B | A |
| 117 | B | A | B | A |
| 118 | C | A | B | A |
| 119 | B | B | B | A |
| 120 | B | A | A | A |
| 121 | C | B | B | A |
| 122 | B | B | B | B |
| 123 | B | A | B | A |
| 124 | C | B | D | B |
| 125 | B | A | A | A |
| 126 | B | B | A | A |
| 127 | C | C | A | A |
| 128 | B | B | A | A |
| 129 | C | C | A | A |
| 130 | C | B | C | B |
| 131 | C | B | B | A |
| 132 | C | B | B | A |
| 133 | C | C | B | A |
| 134 | A | A | A | A |
| 135 | B | B | A | A |
| 136 | C | C | D | C |
| 137 | B | A | B | A |
| 138 | A | A | A | A |
| 139 | A | A | B | A |
| 140 | A | A | B | A |
| 141 | 0 | B | C | A |
| 142 | A | A | B | A |
| 143 | B | A | B | B |
| 144 | A | A | B | A |
| 145 | B | A | B | A |
| 146 | A | B | C | B |
| 147 | A | A | A | A |
| 148 | A | B | B | A |
| 148 | A | A | B | A |
| 148a | A | A | C | B |
| 148b | A | A | C | B |
| 149 | B | B | D | C |
| 150 | B | A | B | A |
| 151 | C | A | C | A |
| 152 | B | B | B | A |
| 153 | B | B | C | A |
| 154 | B | A | B | A |
| 155 | B | A | B | A |
| 156 | A | B | A | A |
| 157 | C | A | C | A |
| 158 | B | A | A | A |
| 159 | C | A | C | A |
| 160 | B | A | B | A |
| 161 | B | A | A | A |
| 162 | C | B | A | A |
| 163 | B | A | B | A |
| 164 | C | A | 0 | A |
| 165 | A | A | A | A |
| 166 | B | C | B | B |
| 167 | B | B | A | A |
| 168 | B | A | A | A |
| 169 | A | A | A | A |
| 169a | A | A | A | A |
| 169b | B | A | B | A |
| 170 | A | A | A | A |
| 171 | A | A | A | A |
| 172 | A | A | A | A |
| 173 | B | A | A | A |
| 174 | A | A | B | A |
| 175 | B | A | A | A |
| 176 | B | A | B | A |
| 177 | B | B | B | B |
| 178 | B | B | B | B |
| 179 | C | A | C | A |
| 180 | A | A | A | A |
| 181 | A | A | A | A |
| 182 | B | A | B | A |
| 183 | B | A | B | A |
| 184 | A | A | B | A |
| 185 | C | C | A | A |
| 186 | B | A | B | A |
| 187a | A | A | A | A |
| 187b | A | A | A | A |
| 188 | A | A | B | A |
| 189 | A | A | A | A |
| 190 | A | A | B | A |
| 191 | B | A | B | A |
| 192 | B | A | B | A |
| 193 | C | C | C | A |
| 194 | C | B | B | A |
| 195 | C | B | C | A |
| 196 | C | A | C | A |
| 197 | A | A | B | A |
| 198 | B | A | B | A |
| 200 | B | B | B | A |
| 201 | B | A | B | B |
| 202 | C | C | B | A |
| 203 | C | B | B | A |
| 204 | B | A | B | A |
| 205 | B | A | B | A |
| 206 | C | A | B | A |
| 207 | B | B | B | A |
| 208 | C | A | C | A |
| 209 | B | A | B | A |
| 210 | B | A | B | A |
| 211 | C | A | C | A |
| 212 | C | C | B | A |
| 213 | D | C | B | A |
| 214 | C | B | B | A |
| 215 | B | A | C | A |
| 216 | A | A | B | A |
| 217 | A | A | A | A |
| 218 | C | B | C | B |
| 219 | B | A | A | A |
| 220 | B | B | B | B |
| 221 | C | A | C | A |
| 222 | B | A | B | A |
| 223 | C | B | B | A |
| 224 | B | B | B | A |
| 225 | C | B | B | A |
| 226 | C | B | B | A |
| 227 | C | C | B | A |
| 228 | C | C | A | A |
| 229 | B | A | A | A |
| 230 | C | A | B | A |
| 231 | B | B | A | A |
| 232 | B | B | C | B |
| 233 | C | A | C | A |
| 234 | A | A | A | A |
| 235 | A | A | A | A |
| 236 | A | A | A | A |
| 237 | A | A | B | A |
| 238 | B | A | B | A |

DC₅₀ values:
A: DC₅₀ < 50 nM;
B: 50 nM ≤ DC₅₀ < 200 nM;
C: 200 nM ≤ DC₅₀ < 1000 nM;
D: 1000 nM ≤ DC₅₀ ;
Eₘₐₓ values:
A: Eₘₐₓ < 10;
B: 10 ≤ Eₘₐₓ < 20;
C: 20 ≤ Eₘₐₓ < 50;
D: 50 ≤ Eₘₐₓ

### In Vivo Efficacy of Exemplary PTPN2 Degrader in MC38 Tumor Model (Compound 187b) Mice

All experiments were conducted in compliance with AbbVie's Institutional Animal Care and Use Committee and the National Institutes of Health Guide for Care and Use of Laboratory Animals guidelines in a facility accredited by the Association for the Assessment and Accreditation of Laboratory Animal Care. C57Bl/6 female mice were obtained from Charles River (Wilmington, MA). The mice were group-housed 10 per cage. Food and water were available ad libitum. Animals were acclimated to the animal facilities for a period of at least one week prior to commencement of experiments. Animals were tested in the light phase of a 12-hour light: 12-hour dark schedule (lights on 0600 hours).

### Tumor Cell Inoculation and Treatments

Cells were grown to passage 3 in vitro. A total of 1×10⁵ viable MC-38 cells were inoculated subcutaneously into the right flank of female C57Bl/6 mice (7-12 weeks old) on Day 0. The injection volume was 0.1 mL and was composed of a 1:1 mixture of S-MEM and Matrigel® (Corning, NY, USA). Tumors were size matched on Day 14 and the mice had a mean body weight of ˜21 g. The mean tumor volume (TV) at size match was approximately 139±45 mm³. Following size match, treatments were initiated on the same day. Dosing of mice with Compound 187b (Example 187b) was conducted intravenously, weekly (QW) for 3 weeks. Mice were dosed (10 mg/kg/dose) with either Compound 187b or vehicle controls (n=10 mice/group). Compound 187b was formulated in 5% DMSO+10% PEG400+85% (35% HP-β-CD in water) and was dosed at 5 mL/kg. Anti-PD-1 and isotype antibodies were dosed at 5 mg/kg, twice at 4 day intervals (Q4Dx3). Antibodies were formulated in PBS and dosed at 10 mL/kg. Tumor volume was calculated three times weekly. Measurements of the length (L) and width (W) of the tumor were taken via electronic caliper and the volume was calculated according to the following equation: V=L×W²/2 using Study Director Version 3.1.399.22 (Study log Systems, Inc, CA, USA). Mice were euthanized when tumor volume was ≤3000 mm³ or skin ulcerations occurred. Tumor growth inhibition (TGI) was calculated as TGI=1−(Mean TV_{Timepoint (Treatment)}/Mean TV_{Timepoint (Vehicle)} for each timepoint that tumor volumes were measured. Reported TGI_{Max} is the largest TGI value for any timepoint that tumors volumes were collected for that treatment group.

### Results

PTPN2 is a regulator of anti-tumor immune responses through both tumor- and immune-cell intrinsic effects. Therefore, compounds capable of inducing the specific degradation of PTPN2/1B were created and are assessed for their ability to elicit tumor growth inhibition and anti-tumor effects in an in vivo syngeneic mouse tumor model. Mice were inoculated on their hind flank with the murine colon adenocarcinoma, MC-38. Following two weeks of tumor cell growth, mice began intravenous weekly treatment for 3 weeks with either the vehicle or the formulated Compound 187b along with either anti-PD-1 or isotype antibody co-treatment through intraperitoneal dosing every 4 days for 2 doses.

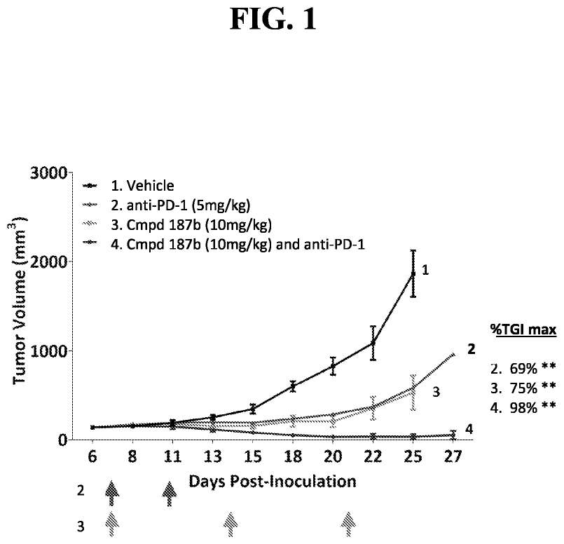

FIG. 1 shows tumor growth inhibition of MC-38 tumor-bearing mice treated with Compound 187b monotherapy (green arrows; QWx3) and in combination with a-PD-1 therapy (red arrows; Q4Dx2). Each point on the curve represents the mean of 10 tumor volume of 10 mice. Error bars depict the standard error of the mean. **=p<0.01.

Compound 187b was well tolerated, without obvious adverse health events. Similar to anti-PD-1 monotherapy treated mice, within 7-10 days of monotherapy treatment with Compound 187b, apparent tumor stasis and shrinkage was observed. Eventually, 10% of mice treated with Compound 1876 achieved complete cures, and an overall TGI_{Max} of 75%, compared to 30% cures and TGI_{Max} of 69% for a-PD-1 treated mice (FIG. 1). Enhanced tumor regression was observed in mice treated with the combination of anti-PD-1 antibody with Compound 187b with 90% of mice in this group achieving cures and an overall TGI_{Max} of 98% (FIG. 1).

These results indicate that compounds of this disclosure can induce the specific degradation of PTPN2/1B in vivo and be useful in various therapeutic applications, including treatment of cancer and cancer immunotherapy.

### EQUIVALENTS AND INCORPORATION BY REFERENCE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 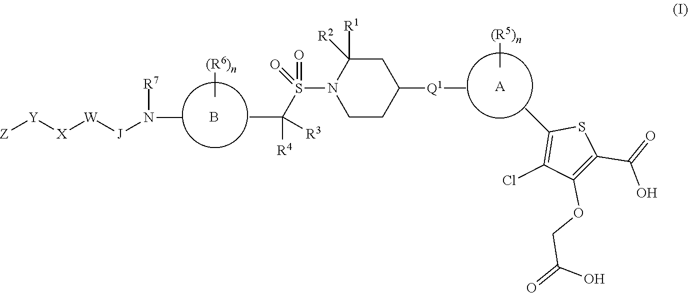
or a pharmaceutically acceptable salt thereof:
wherein:
R¹ and R² are independently hydrogen or C1-C6 alkyl; or R¹ and R², together with the carbon atom to which they are attached, form a C3-C4 cycloalkyl or a 3-4 membered heterocyclyl;
R³ and R⁴ are independently hydrogen, C1-C6 alkyl, or phenyl; or R³ and R⁴, together with the carbon atom to which they are attached, form a C3-C4 cycloalkyl or a 3-4 membered heterocyclyl;
each R⁵ and R⁶ is independently halogen, C1-C6 alkyl, C1-C6 alkoxy, C1-C6 haloalkyl, C1-C6 haloalkoxy, hydroxyl, or —NR^{8A}R^{2B},
R⁷ is hydrogen, C1-C6 alkyl, or C3-C4 cycloalkyl; or wherein R⁷ and R^{C} of W, together with the nitrogen atoms to which they are attached, form a 5-6 membered heterocyclyl;
R^{8A} and R^{8B} are independently hydrogen or C1-C6 alkyl;
Ring A and Ring B are independently phenyl or 6-membered heteroaryl;
n and m are independently 0, 1, or 2;
Q¹ is O or NR⁹;
R⁹ is hydrogen or C1-C6 alkyl;
J is a bond or —C(═O)—;
W and Y are independently selected from: —(CR^{A}R^{B})p-*, —((CR^{A}R^{B})pO)t-*, —(O(CR^{A}R^{B})p)t-*, —((CR^{A}R^{B})pO)t-(CR^{A}R^{B})p-*, —((CR^{A}R^{B})₂O(CR^{A}R^{B})₂))p-*, —NR^{C}(CR^{A}R^{B})p-*, —(CR^{A}R^{B})pNR^{C}(C═O)(CR^{A}R^{B})p-*, —(CR^{A}R^{B})p(C═O)NR^{C}(CR^{A}R^{B})p-*, —(CR^{A}R^{B})pNR^{C}-*, —(CR^{A}R^{B})pNR^{C}(C═O)(CR^{A}R^{B})p-O—*, —(CR^{A}R^{B})p(C═O)NR^{C}(CR^{A}R^{B})s-O—*, —(CR^{A}R^{B})pNR^{C}(C═O)(CR^{A}R^{B})p-NR^{C}—*, —(CR^{A}R^{B})p(C═O)NR^{C}(CR^{A}R^{B})s-NR^{C}—*; —NR^{C}(CR^{A}R^{B})s-NR^{C}(C═O)(CR^{A}R^{B})p-*, —NR^{C}(CR^{A}R^{B})p)C≡C—*, —C≡C((CR^{A}R^{B})p)NR^{C}—*, —(C═O) (CR^{A}R^{B})p-O—*, —O—*, —O—(CR^{A}R^{B})p-(C═O)—*, —(C═O)(CR^{A}R^{B})p-NR^{C}—*, —NR^{C}—(CR^{A}R^{B})p-(C═O)—*, —(C═O)(CR^{A}R^{B})p)-*, —(C═O)(CR^{A}R^{B})p-O—(CR^{A}R^{B})p-*, and —((CR^{A}R^{B})p)(C═O)—*, wherein the asterisk represents the point of attachment of W to X or the point of attachment of Y to Z;
each p is independently 0, 1, 2, 3, 4, or 5;
each s is independently 2, 3, 4, or 5;
each t is independently 1, 2, or 3;
each R^{A} and R^{B} is independently hydrogen, fluoro, or C1-C6 alkyl; or R^{A} and R^{B}, together with the carbon atom to which they are attached, form a C3-C4 cycloalkyl;
each R^{C} is independently hydrogen or C1-C6 alkyl; or wherein R^{C} of W and R⁷, together with the nitrogen atoms to which they are attached, form a 5-6 membered heterocyclyl;
X is a bond, C3-C6 cycloalkyl, phenyl optionally substituted with 1-3 independently selected halogen atoms, 3 to 10 membered heterocyclyl optionally substituted with 1-3 substituents independently selected from halogen, C1-C6 alkyl, and hydroxyl, or 5 to 10 membered heteroaryl optionally substituted with 1-3 independently selected halogen atoms;
Z is selected from the group consisting of: 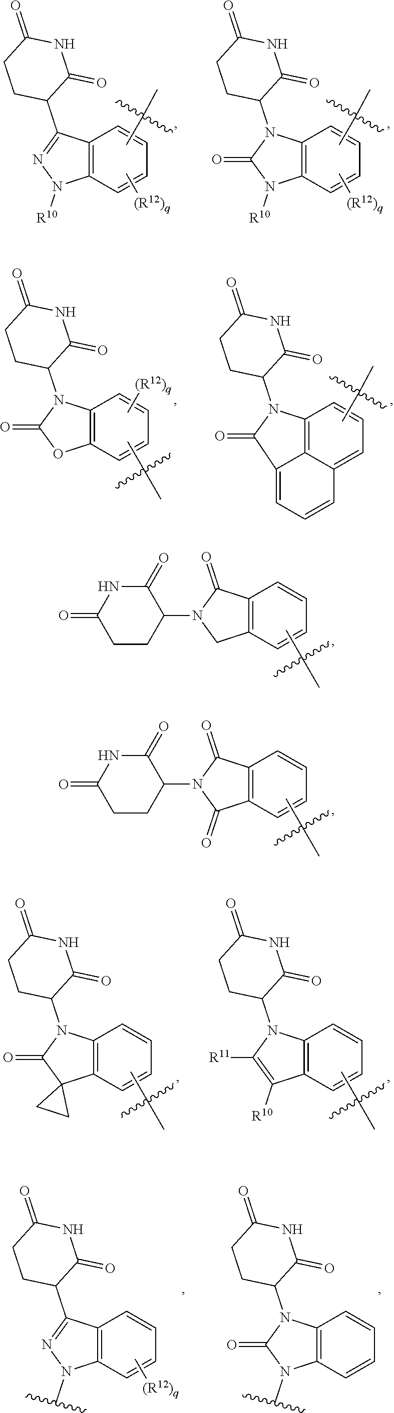 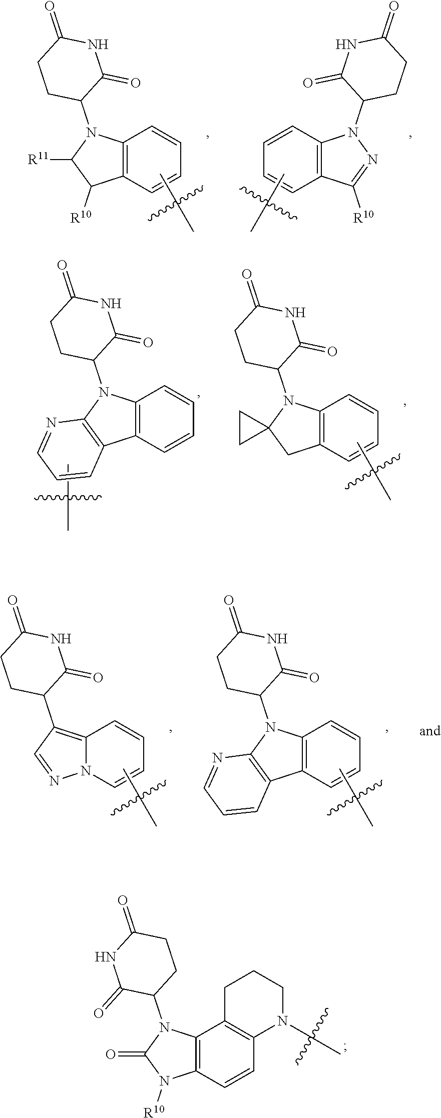
R¹⁰ is hydrogen, C1-C6 alkyl optionally substituted with C1-C6 alkoxy, C1-C6 haloalkyl, C3-C6 cycloalkyl, or 4-6 membered heterocyclyl;
R¹¹ is hydrogen or C1-C6 alkyl;
each R¹² is independently halogen, cyano, C1-C6 alkyl, C1-C6 haloalkyl, C1-C6 alkoxy, or C3-C5 cycloalkoxy; and
q is 0, 1, or 2.

2. The compound of claim 1, wherein Ring A is phenyl.

3-10. (canceled)

11. The compound of claim 1, wherein R¹ and R² are independently hydrogen or C1-C6 alkyl.

12-15. (canceled)

16. The compound of claim 1, wherein R³ and R⁴ are independently hydrogen, C1-C6 alkyl, or phenyl.

17. (canceled)

18. The compound of claim 1, wherein Ring B is phenyl.

19-23. (canceled)

24. The compound of claim 1, wherein R⁷ is hydrogen or C1-C3 alkyl.

25. (canceled)

26. The compound of claim 1, wherein W is: —(CR^{A}R^{B})p-*, —((CR^{A}R^{B})pO)t-*, —((CR^{A}R^{B})pO)t-(CR^{A}R^{B})p-*, —((CR^{A}R^{B})₂O(CR^{A}R^{B})₂))p-*, —(CR^{A}R^{B})pO-*, —O(CR^{A}R^{B})p-*, or —(CR^{A}R^{B})pNR^{C}-*.

27-32. (canceled)

33. The compound of claim 1, wherein Y is: —(CR^{A}R^{B})p-*, —(C═O)(CR^{A}R^{B})p-O—*, —(C═O)(CR^{A}R^{B})p-NR^{C}—*, —(C═O)(CR^{A}R^{B})p-*, —(CR^{A}R^{B})p(C═O)NR^{C}(CR^{A}R^{B})p-*, —(CR^{A}R^{B})pNR^{C}(C═O)(CR^{A}R^{B})p-*, —(CR^{A}R^{B})pNR^{C}(C═O)(CR^{A}R^{B})pO—*, —(CR^{A}R^{B})pNR^{C}(C═O)(CR^{A}R^{B})p-NR^{C}—*, —C≡C((CR^{A}R^{B})p)NR^{C}—*, —(CR^{A}R^{B})pO-*, —O(CR^{A}R^{B})p-*, or —(CR^{A}R^{B})pNR^{C}—*.

34-47. (canceled)

48. The compound of claim 1, wherein X is selected from:
a bond;
4-7 membered monocyclic heterocyclyl optionally substituted with 1-3 substituents independently selected from halogen, C1-C6 alkyl, and hydroxyl;
azetidinyl, piperidinyl, and piperazinyl optionally substituted with 1-3 substituents independently selected from halogen, C1-C6 alkyl, and hydroxyl; 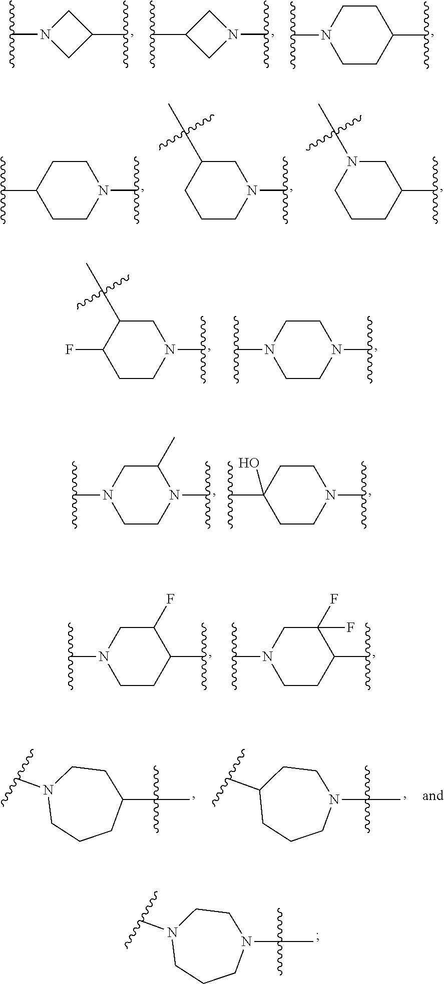
6-10 membered bicyclic heterocyclyl optionally substituted with 1-3 substituents independently selected from halogen, C1-C6 alkyl, and hydroxyl;
6-10 membered bicyclic fused heterocyclyl optionally substituted with 1-3 substituents independently selected from halogen, C1-C6 alkyl, and hydroxyl;
7-10 membered bicyclic spiroheterocyclyl optionally substituted with 1-3 substituents independently selected from halogen, C1-C6 alkyl, and hydroxyl; 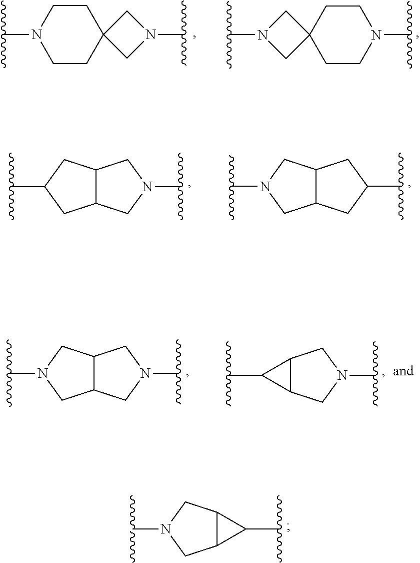
cyclohexyl;
phenyl optionally substituted with 1-3 independently selected halogen atoms;
5-6 membered heteroaryl optionally substituted with 1-3 independently selected halogen atoms;
1,2,3-triazolyl, pyrazolyl, and imidazolyl optionally substituted with 1-3 independently selected halogen atoms, and

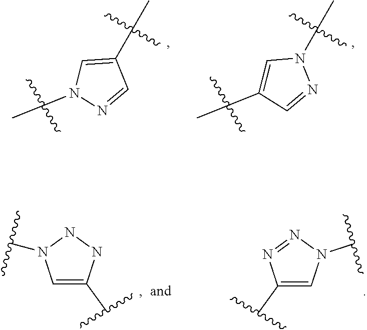 [SMILES: *c1cnn(*)c1 ; *c1cnn(*)c1 ; *C1=CN(*)NN1 ; *c1cn(*)nn1]

49-61. (canceled)

62. The compound of claim 1, wherein;
X is 4-10 membered heterocyclyl optionally substituted with 1-3 substituents independently selected from halogen, C1-C6 alkyl, and hydroxyl; and
W is —(CR^{A}R^{B})p¹-*, —(CR^{A}R^{B})p¹O—*, —O(CR^{A}R^{B})p¹-*, —NR^{C}(CR^{A}R^{B})p¹-*, or (CR^{A}R^{B})p¹-NR^{C}—*, wherein p¹ is 0, 1, 2, 3, 4, or 5.

63. (canceled)

64. The compound of claim 62, wherein Y is: —(CR^{A}R^{B})p²-*, —(C═O)(CR^{A}R^{B})p²-O—*, —(C═O)(CR^{A}R^{B})p²-NR^{C}—*, —(C═O)(CR^{A}R^{B})p²-*, —(CR^{A}R^{B})p²-O—*, —O(CR^{A}R^{B})p²-*, —NR^{C}—(CR^{A}R^{B})p²-*, —(CR^{A}R^{B})p²-NR^{C}—*, or —(CR^{A}R^{B})p²(C═O)NR^{C}(CR^{A}R^{B})p²-*, wherein p² is 0, 1, 2, 3, 4, or 5.

65-71. (canceled)

72. The compound of claim 62, wherein X is selected from:
4-7 membered monocyclic heterocyclyl optionally substituted with 1-3 substituents independently selected from halogen, C1-C6 alkyl, and hydroxyl;
azetidinyl, piperidinyl, and piperazinyl optionally substituted with 1-3 substituents independently selected from halogen, C1-C6 alkyl, and hydroxyl; 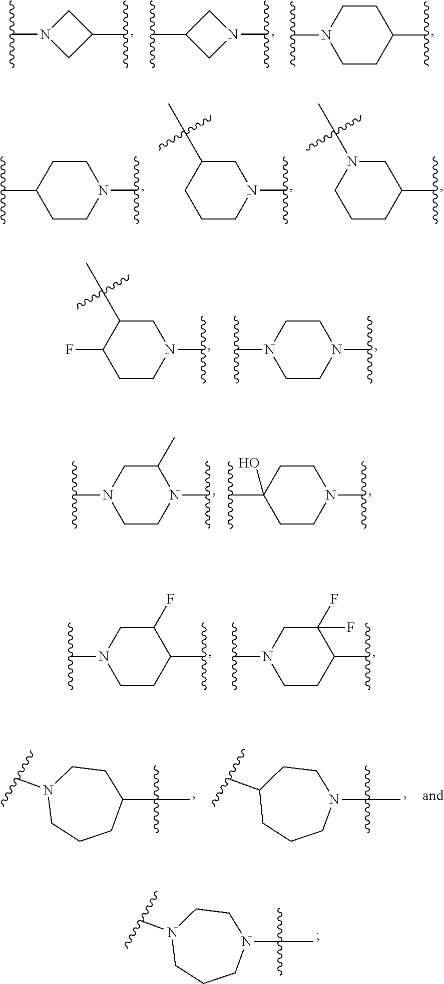
6-10 membered bicyclic heterocyclyl optionally substituted with 1-3 substituents independently selected from halogen, C1-C6 alkyl, and hydroxyl;
6-10 membered bicyclic fused heterocyclyl optionally substituted with 1-3 substituents independently selected from halogen, C1-C6 alkyl, and hydroxyl;
7-10 membered bicyclic spiroheterocyclyl optionally substituted with 1-3 substituents independently selected from halogen, C1-C6 alkyl, and hydroxyl; and

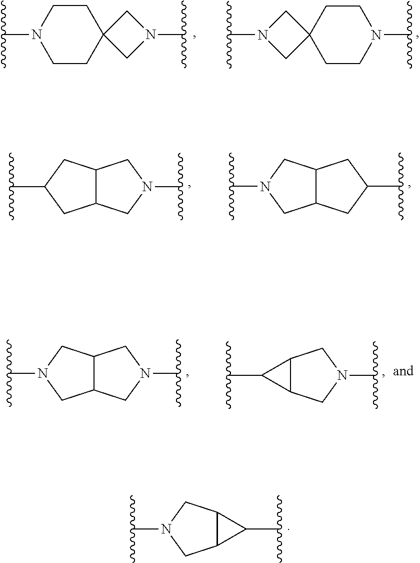

73-101. (canceled)

102. The compound of claim 62, wherein Z is selected from:

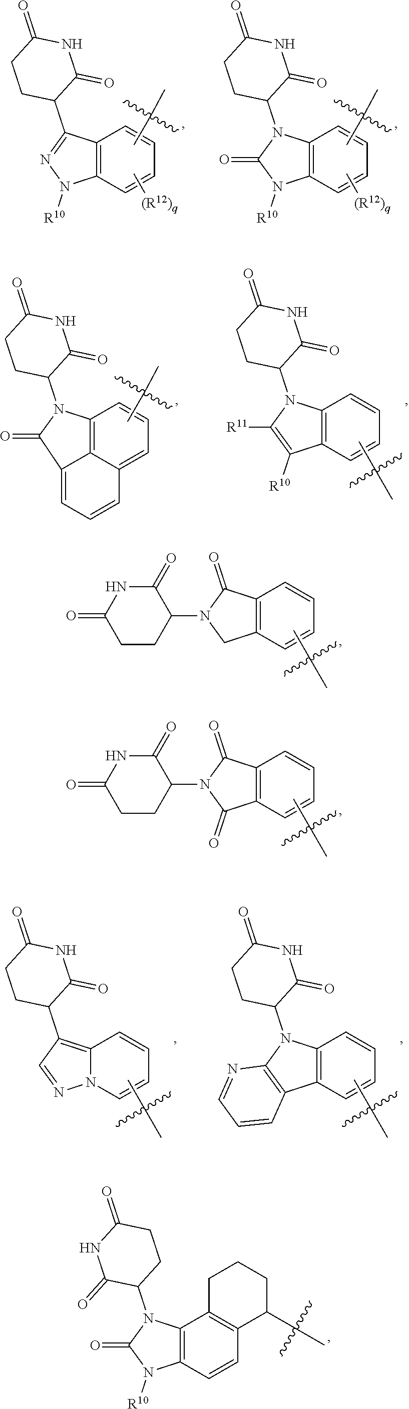

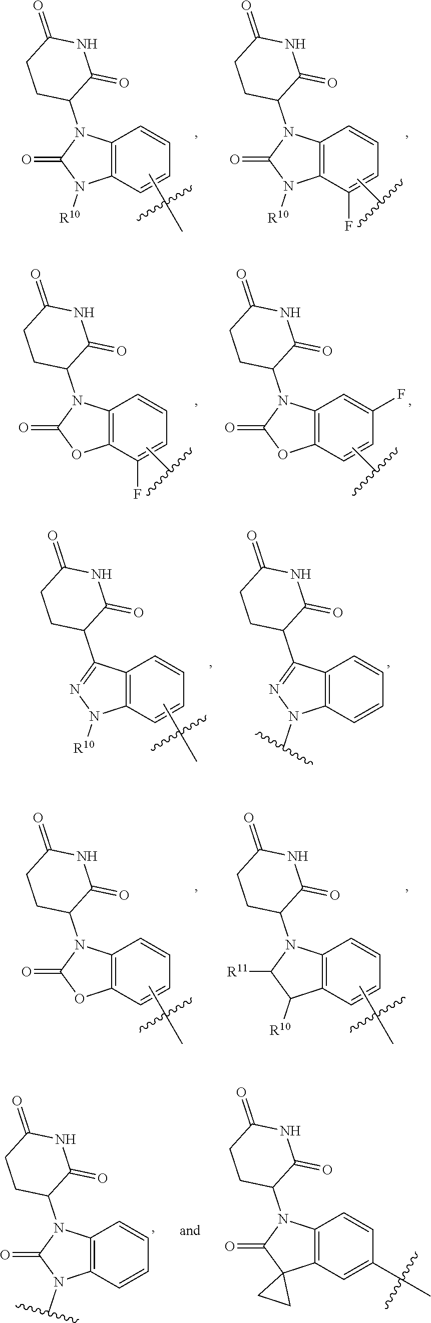

103-115. (canceled)

116. The compound of claim 102, wherein R¹⁰ is C1-C4 alkyl optionally substituted with C1-C6 alkoxy, C1-C6 haloalkyl, C3-C6 cycloalkyl, or 4-6 membered heterocyclyl.

117-121. (canceled)

122. The compound of claim 1, wherein the compound of Formula (I) or a pharmaceutically acceptable salt thereof, is:
Formula (I-a): 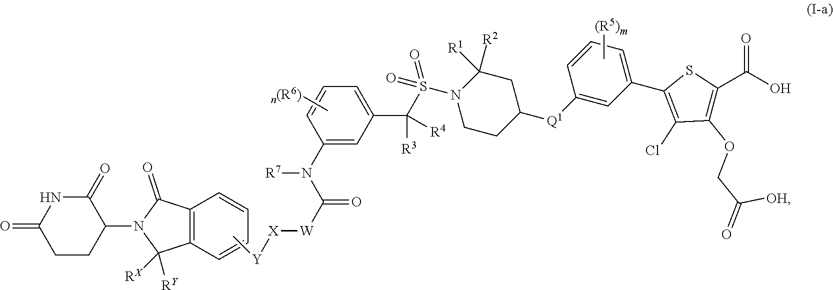
wherein R^{X} and R^{Y} are both H; or R^{X} and R^{Y}, together with the carbon atom to which they are attached, form C═O;
Formula (I-b): 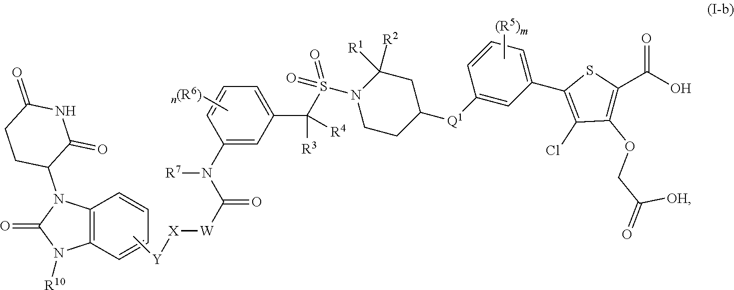
wherein R¹⁰ is hydrogen, C1-C4 alkyl optionally substituted with C1-C6 alkoxy, C1-C6 haloalkyl, C3-C6 cycloalkyl, or 4-6 membered heterocyclyl;
Formula (I-c): 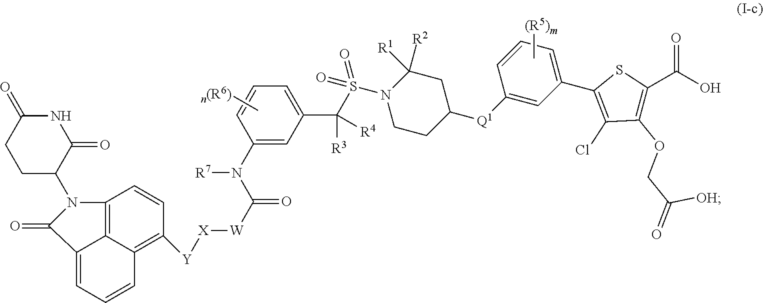
or
Formula (I-d):

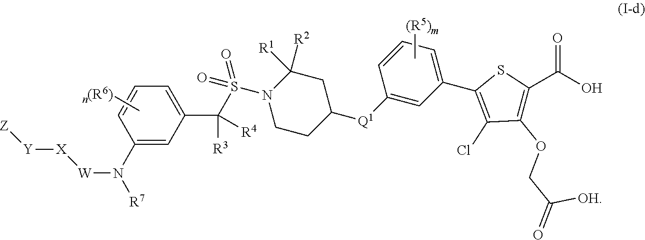

123-130. (canceled)

131. The compound of claim 122, wherein:
m is 0 or 1, and when m is 1, R⁵ is —F;
Q¹ is NH or —O—;
R¹ and R² are both hydrogen or are independently C1-C3 alkyl;
R³ and R⁴ are independently hydrogen, C1-C6 alkyl, or phenyl:
n is 0 or 1, and when n is 1, R⁶ is —F; and
R⁷ is hydrogen.

132-151. (canceled)

152. The compound of claim 122, wherein X is selected from:
4-7 membered monocyclic heterocyclyl optionally substituted with 1-3 substituents independently selected from halogen, C1-C6 alkyl, and hydroxyl;
azetidinyl, piperidinyl, and piperazinyl optionally substituted with 1-3 substituents independently selected from halogen, C1-C6 alkyl, and hydroxyl; 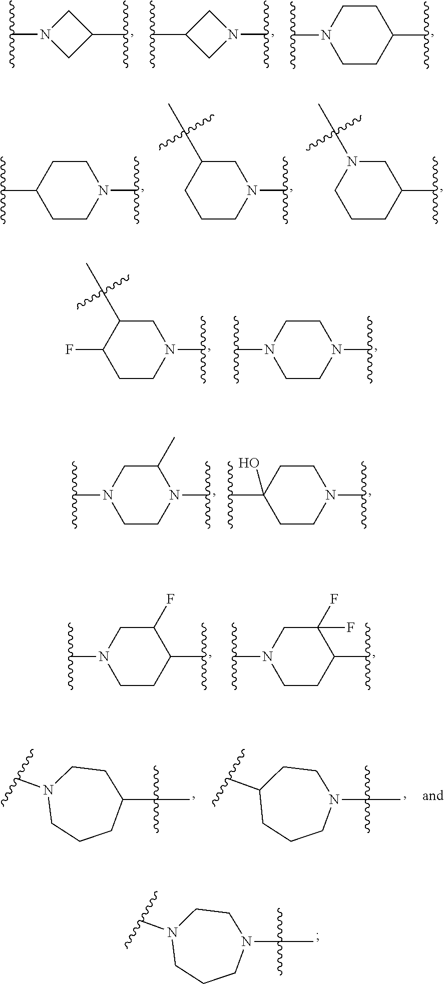
6-10 membered bicyclic heterocyclyl optionally substituted with 1-3 substituents independently selected from halogen, C1-C6 alkyl, and hydroxyl;
6-10 membered bicyclic fused heterocyclyl optionally substituted with 1-3 substituents independently selected from halogen, C1-C6 alkyl, and hydroxyl;
7-10 membered bicyclic spiroheterocyclyl optionally substituted with 1-3 substituents independently selected from halogen, C1-C6 alkyl, and hydroxyl; and

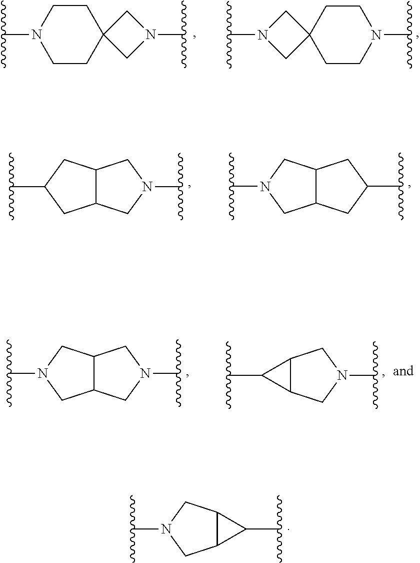

153-177. (canceled)

178. The compound of claim 122, wherein R^{A} and R^{B} are independently hydrogen, fluoro, or C1-C3 alkyl.

179. (canceled)

180. The compound of claim 1, wherein W—X—Y is selected from the group consisting of: 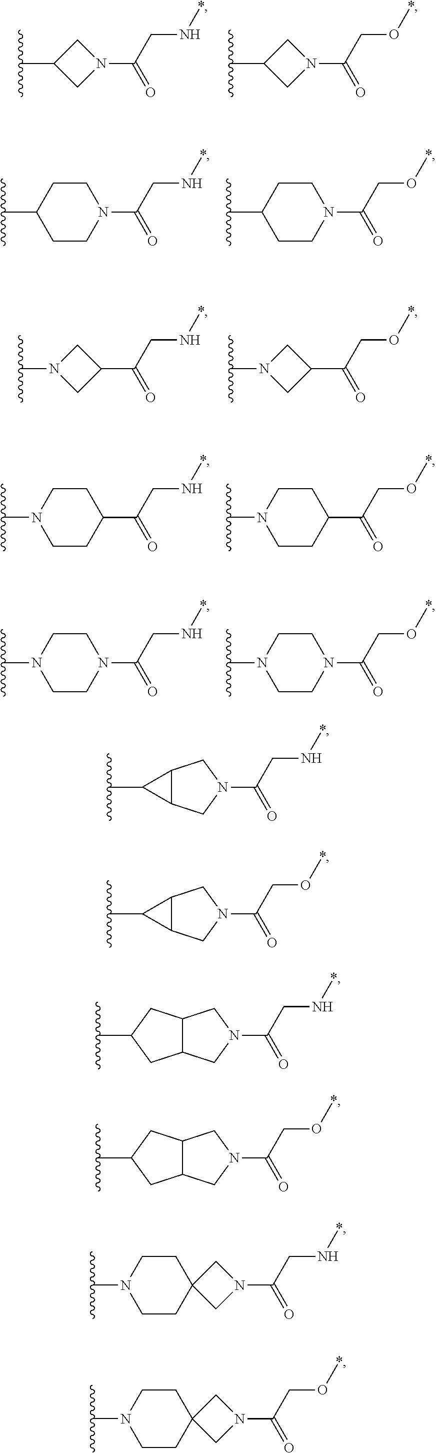 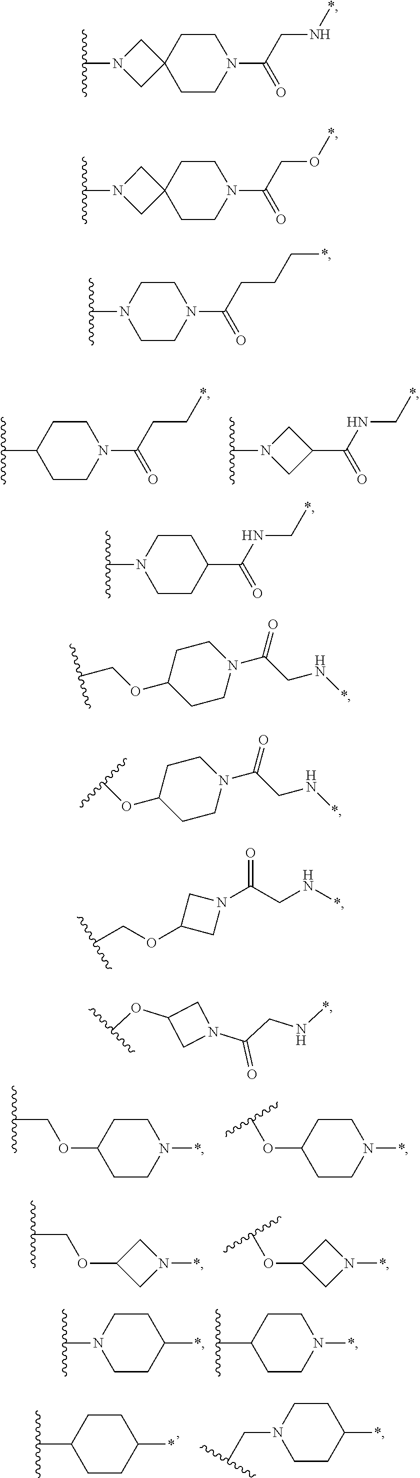 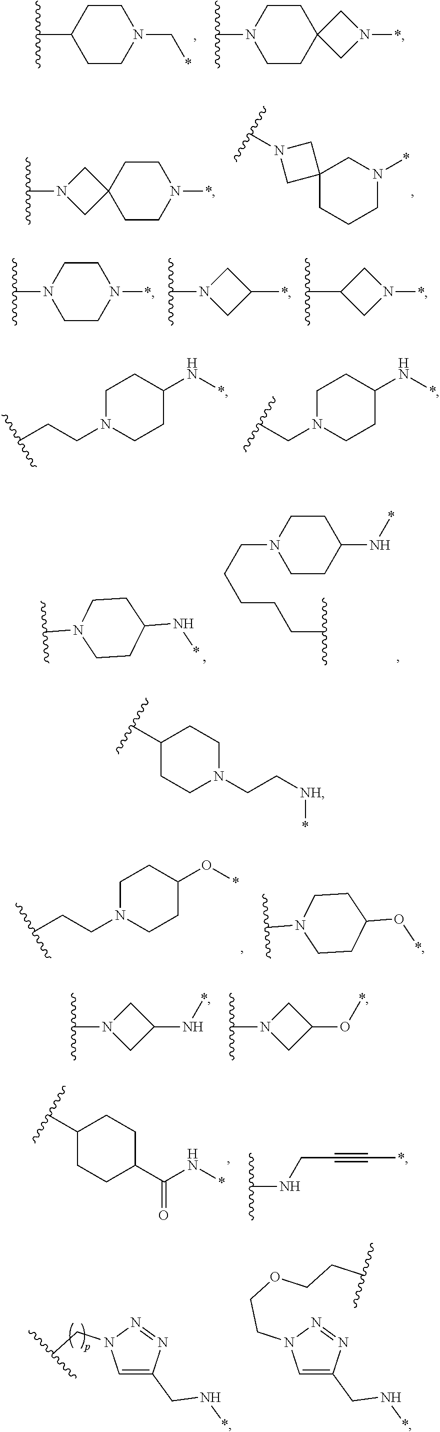 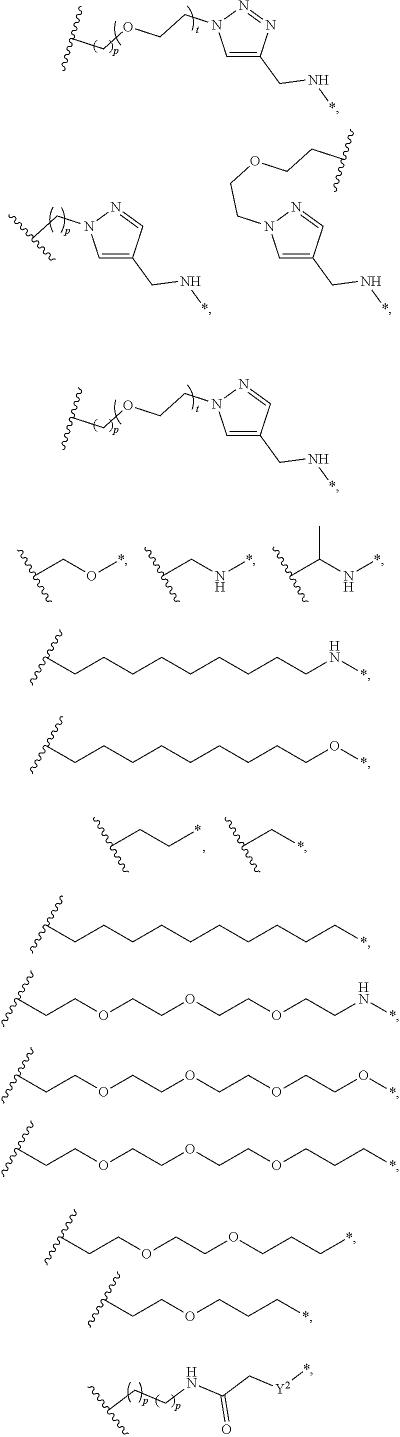 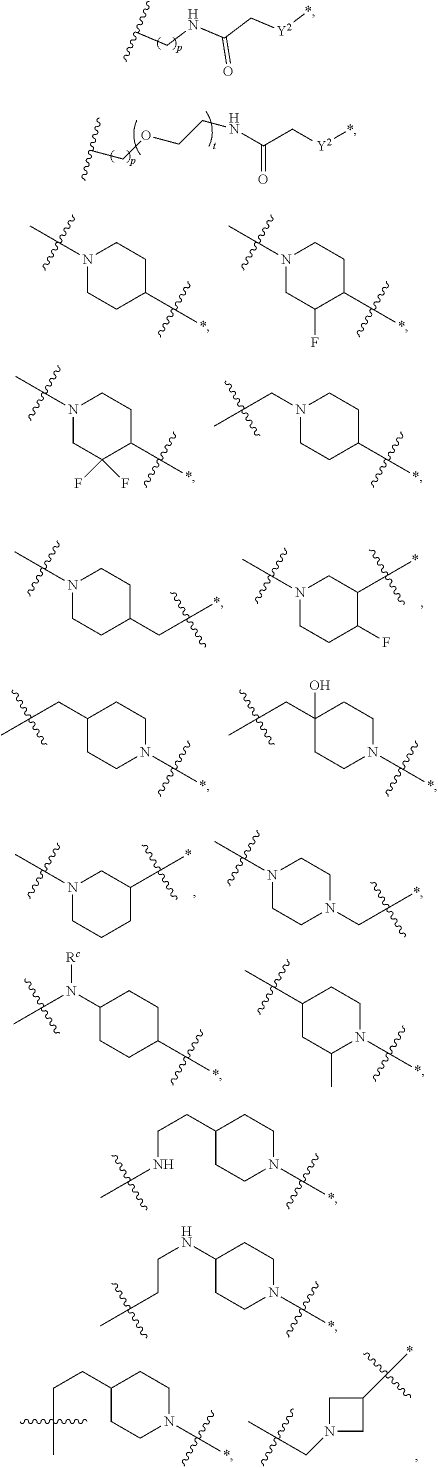 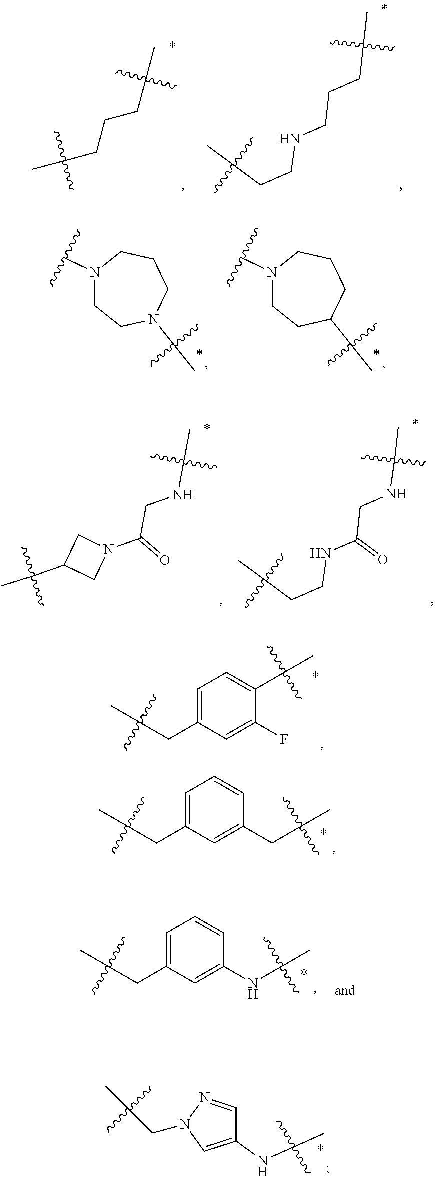
wherein:
Y² is NH, NMe, O, or CH₂;
p is independently 1, 2, 3, 4 or 5; and
the asterisk represents the point of attachment to Z.

181-197. (canceled)

198. The compound of claim 1, wherein the compound of Formula (I) is selected from: 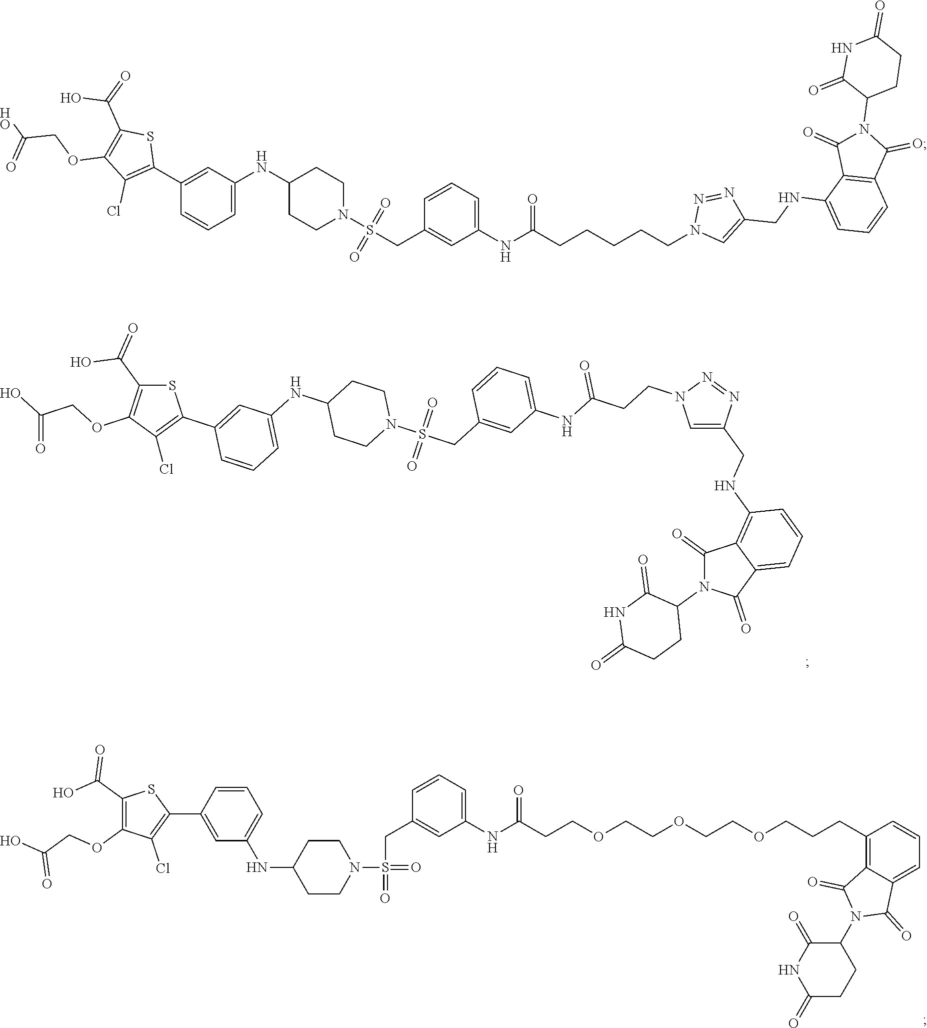 [SMILES: O=C(O)COc1c(C(=O)O)sc(-c2cccc(NC3CCN(S(=O)(=O)Cc4cccc(NC(=O)CCCCCn5cc(CNc6cccc7c6C(=O)N(C6CCC(=O)NC6=O)C7=O)nn5)c4)CC3)c2)c1Cl ; O=C(O)COc1c(C(=O)O)sc(-c2cccc(NC3CCN(S(=O)(=O)Cc4cccc(NC(=O)CCn5cc(CNc6cccc7c6C(=O)N(C6CCC(=O)NC6=O)C7=O)nn5)c4)CC3)c2)c1Cl ; O=C(O)COc1c(C(=O)O)sc(-c2cccc(NC3CCN(S(=O)(=O)Cc4cccc(NC(=O)CCOCCOCCOCCCc5cccc6c5C(=O)N(C5CCC(=O)NC5=O)C6=O)c4)CC3)c2)c1Cl] 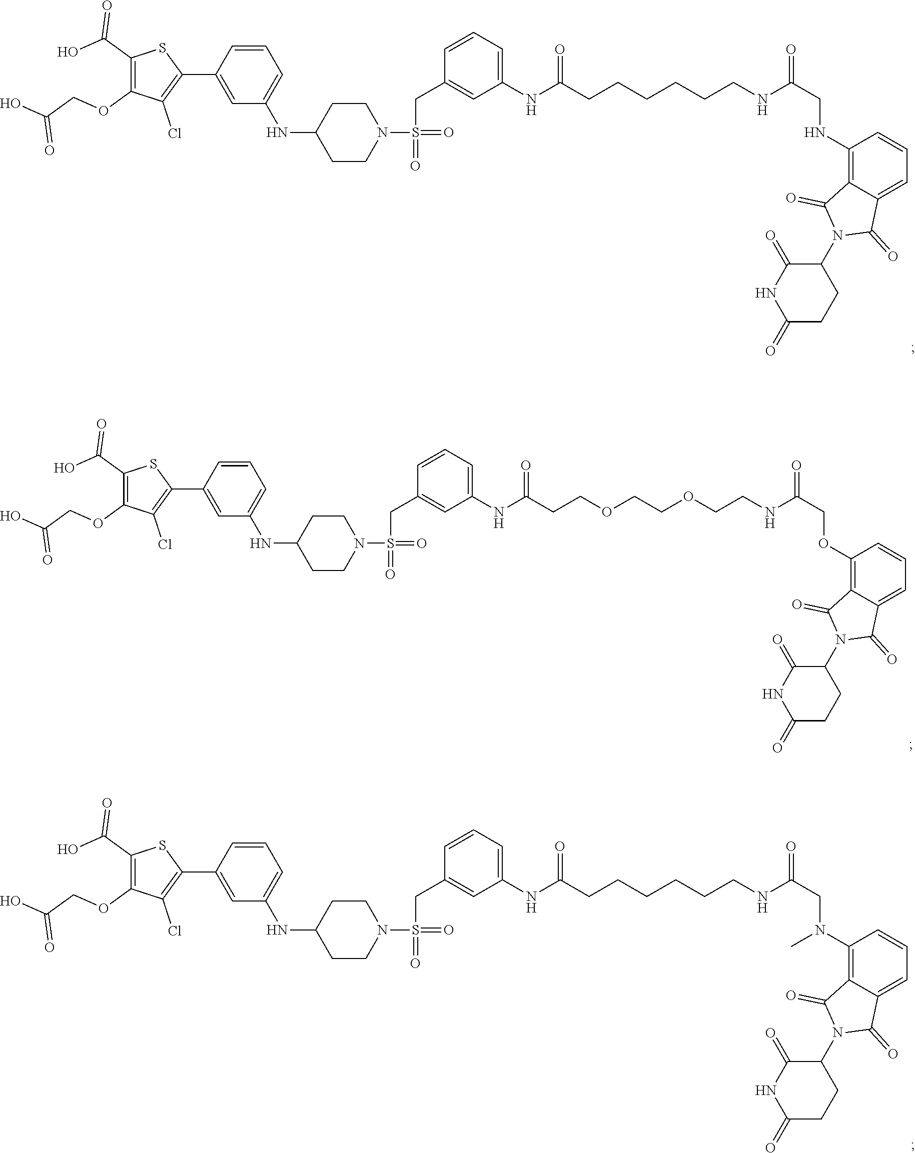 [SMILES: O=C(O)COc1c(C(=O)O)sc(-c2cccc(NC3CCN(S(=O)(=O)Cc4cccc(NC(=O)CCCCCCNC(=O)CNc5cccc6c5C(=O)N(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCOCCOCCNC(=O)COc5cccc6c5C(=O)N(C5CCC(=O)NC5=O)C6=O)c4)CC3)c2)c1Cl ; CN(CC(=O)NCCCCCCC(=O)Nc1cccc(CS(=O)(=O)N2CCC(Nc3cccc(-c4sc(C(=O)O)c(OCC(=O)O)c4Cl)c3)CC2)c1)c1cccc2c1C(=O)N(C1CCC(=O)NC1=O)C2=O] 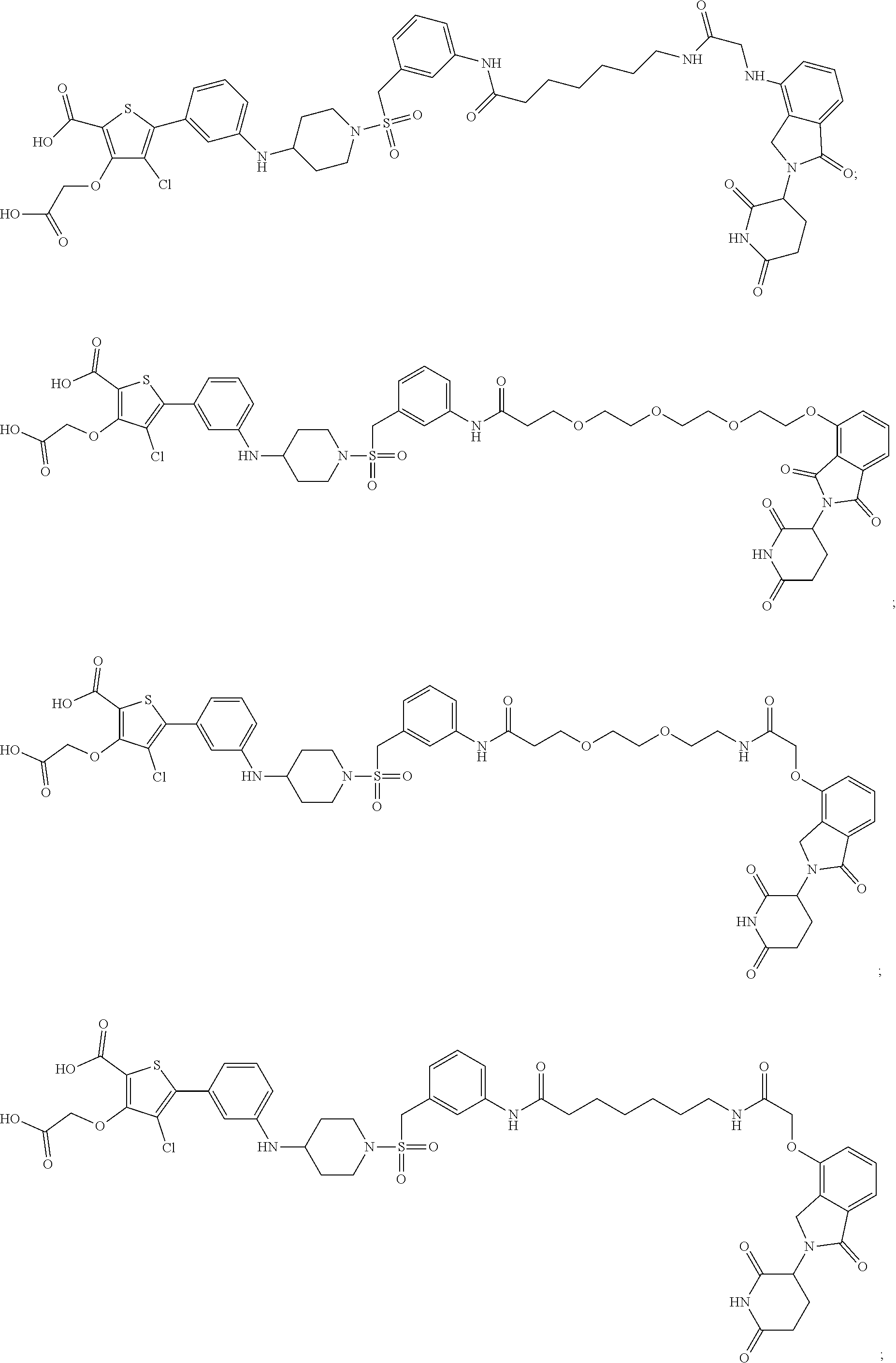 [SMILES: O=C(O)COc1c(C(=O)O)sc(-c2cccc(NC3CCN(S(=O)(=O)Cc4cccc(NC(=O)CCCCCCNC(=O)CNc5cccc6c5CN(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCOCCOCCOCCOc5cccc6c5C(=O)N(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCOCCOCCNC(=O)COc5cccc6c5CN(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCCCCCNC(=O)COc5cccc6c5CN(C5CCC(=O)NC5=O)C6=O)c4)CC3)c2)c1Cl] 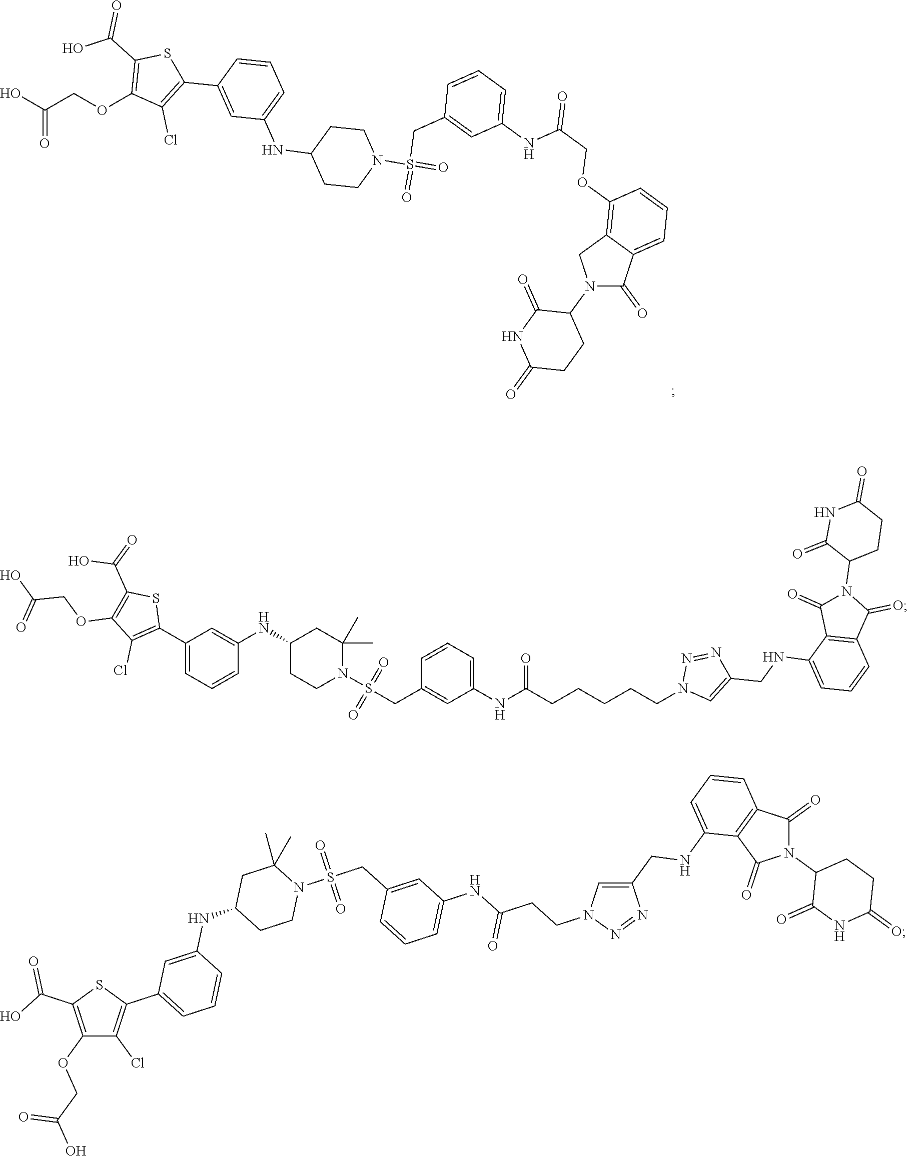 [SMILES: O=C(O)COc1c(C(=O)O)sc(-c2cccc(NC3CCN(S(=O)(=O)Cc4cccc(NC(=O)COc5cccc6c5CN(C5CCC(=O)NC5=O)C6=O)c4)CC3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)CCCCCn2cc(CNc3cccc4c3C(=O)N(C3CCC(=O)NC3=O)C4=O)nn2)c1 ; CC1(C)C[C@@H](Nc2cccc(-c3sc(C(=O)O)c(OCC(=O)O)c3Cl)c2)CCN1S(=O)(=O)Cc1cccc(NC(=O)CCn2cc(CNc3cccc4c3C(=O)N(C3CCC(=O)NC3=O)C4=O)nn2)c1] 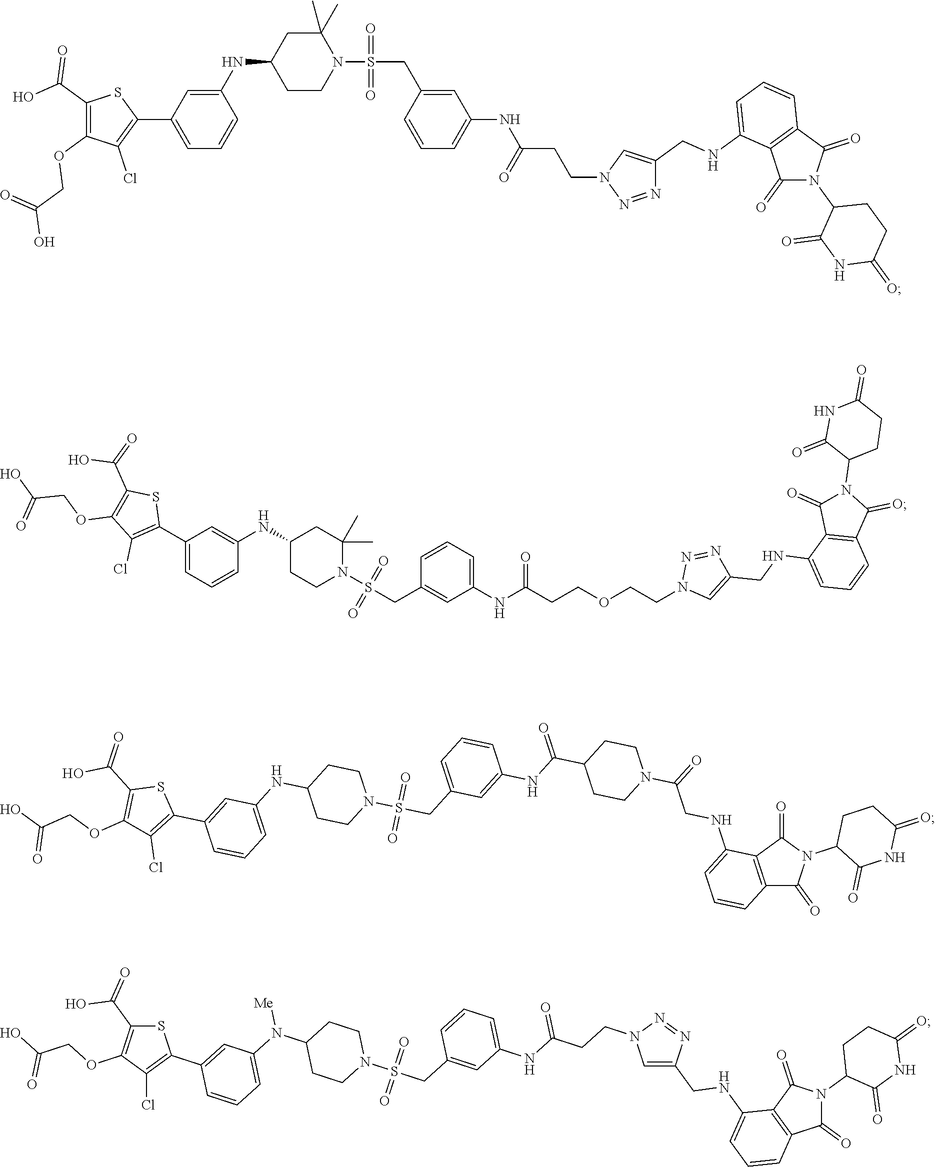 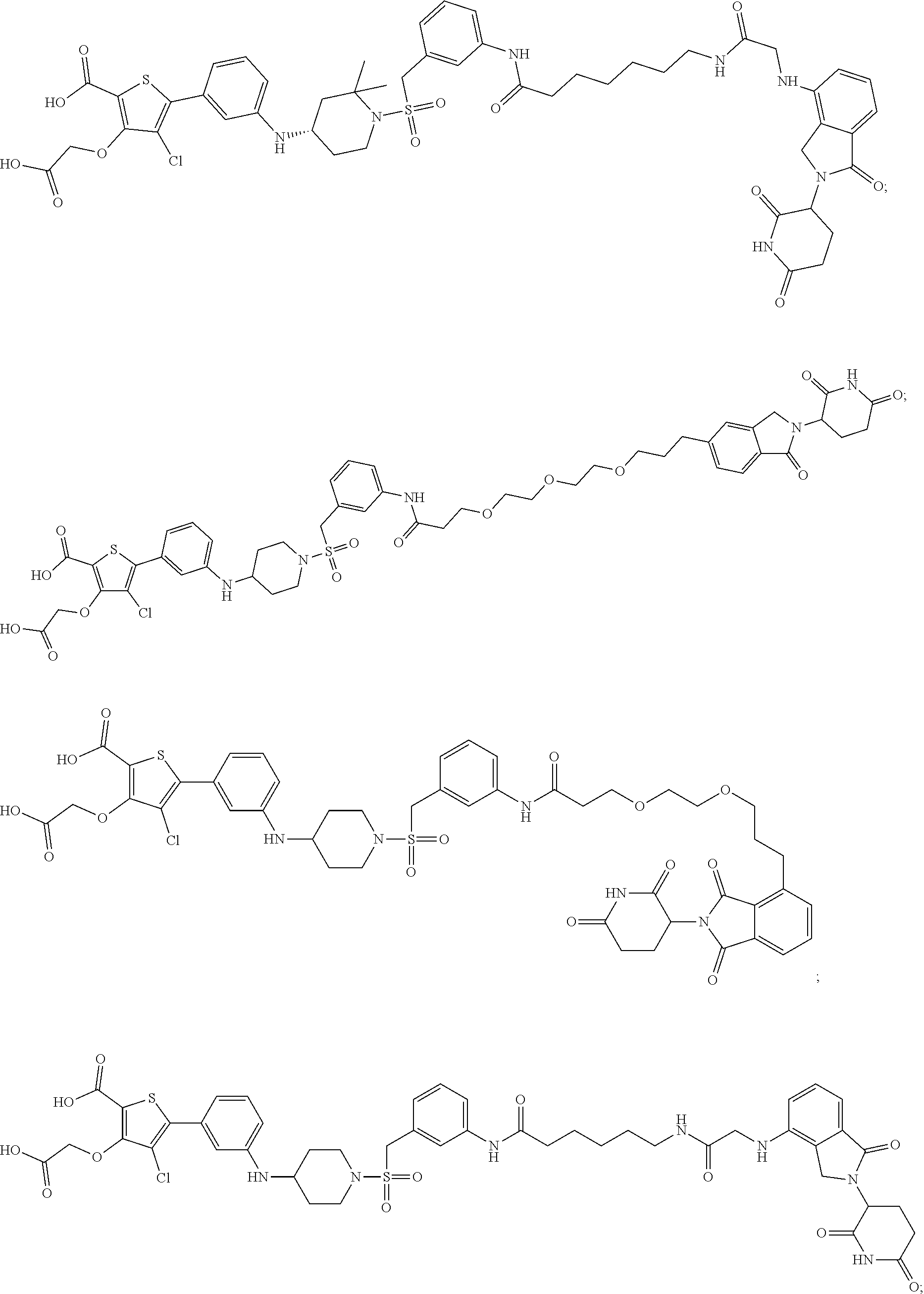 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCCCCCNC(=O)CNc2cccc3c2CN(C2CCC(=O)NC2=O)C3=O)c1 ; O=C(O)COc1c(C(=O)O)sc(-c2cccc(NC3CCN(S(=O)(=O)Cc4cccc(NC(=O)CCOCCOCCOCCCc5ccc6c(c5)CN(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCOCCOCCCc5cccc6c5C(=O)N(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCCCCNC(=O)CNc5cccc6c5CN(C5CCC(=O)NC5=O)C6=O)c4)CC3)c2)c1Cl] 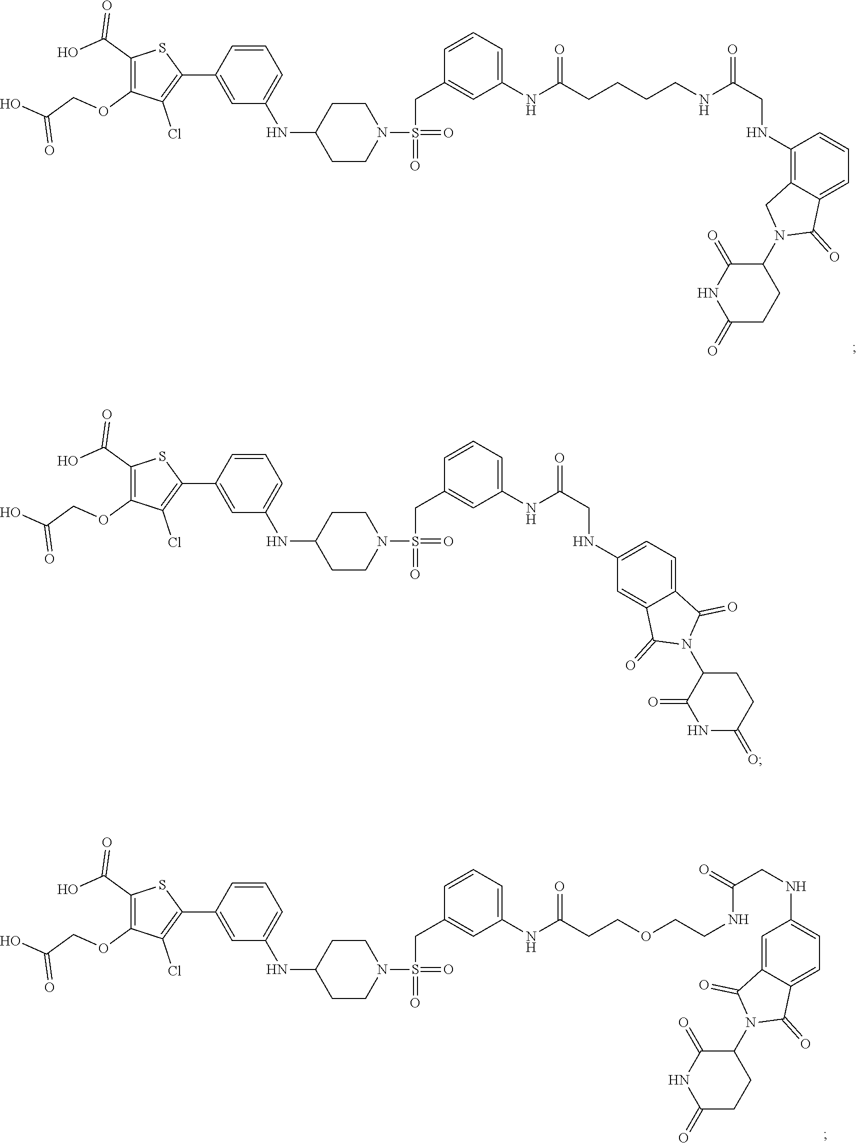 [SMILES: O=C(O)COc1c(C(=O)O)sc(-c2cccc(NC3CCN(S(=O)(=O)Cc4cccc(NC(=O)CCCCNC(=O)CNc5cccc6c5CN(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CNc5ccc6c(c5)C(=O)N(C5CCC(=O)NC5=O)C6=O)c4)CC3)c2)c1Cl ; O=C(O)COc1c(C(=O)O)sc(-c2cccc(NC3CCN(S(=O)(=O)Cc4cccc(NC(=O)CCOCCNC(=O)CNc5ccc6c(c5)C(=O)N(C5CCC(=O)NC5=O)C6=O)c4)CC3)c2)c1Cl] 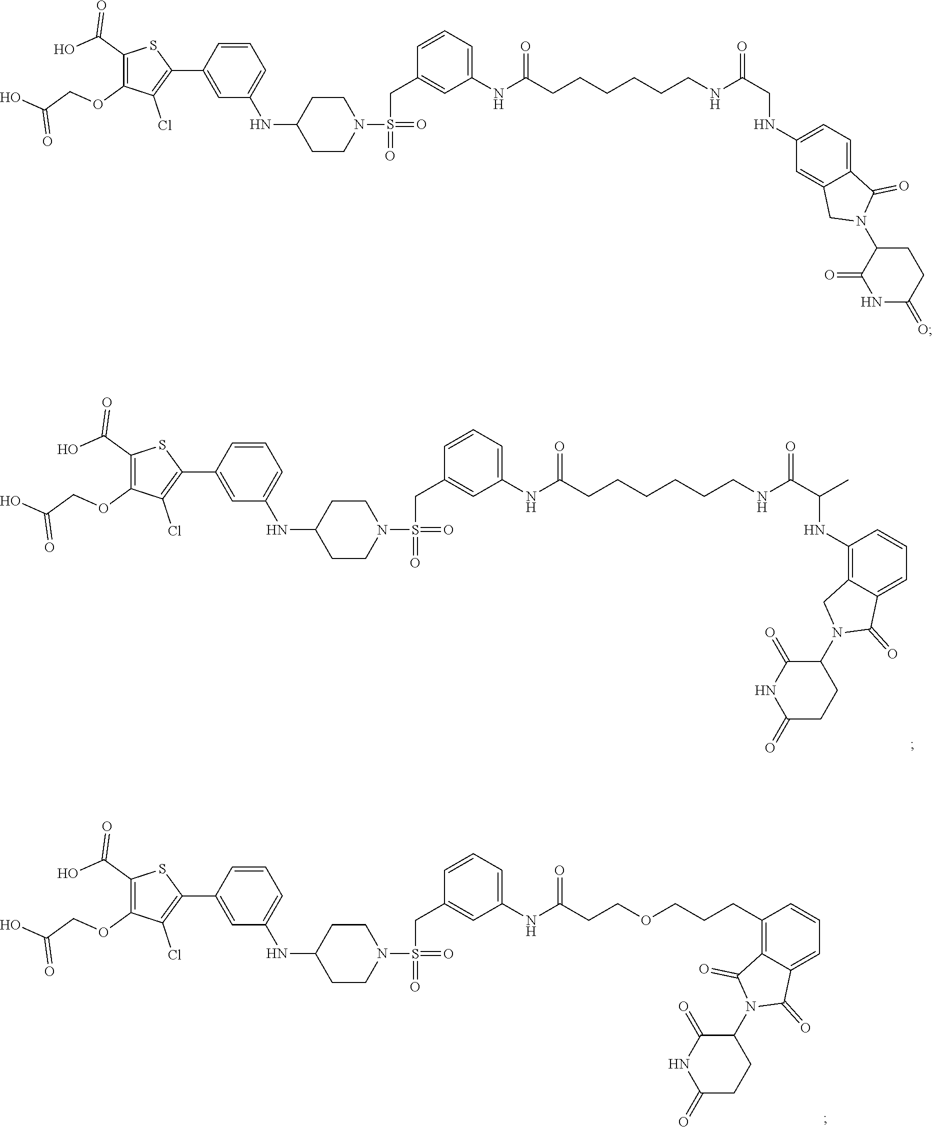 [SMILES: O=C(O)COc1c(C(=O)O)sc(-c2cccc(NC3CCN(S(=O)(=O)Cc4cccc(NC(=O)CCCCCCNC(=O)CNc5ccc6c(c5)CN(C5CCC(=O)NC5=O)C6=O)c4)CC3)c2)c1Cl ; CC(Nc1cccc2c1CN(C1CCC(=O)NC1=O)C2=O)C(=O)NCCCCCCC(=O)Nc1cccc(CS(=O)(=O)N2CCC(Nc3cccc(-c4sc(C(=O)O)c(OCC(=O)O)c4Cl)c3)CC2)c1 ; O=C(O)COc1c(C(=O)O)sc(-c2cccc(NC3CCN(S(=O)(=O)Cc4cccc(NC(=O)CCOCCCc5cccc6c5C(=O)N(C5CCC(=O)NC5=O)C6=O)c4)CC3)c2)c1Cl] 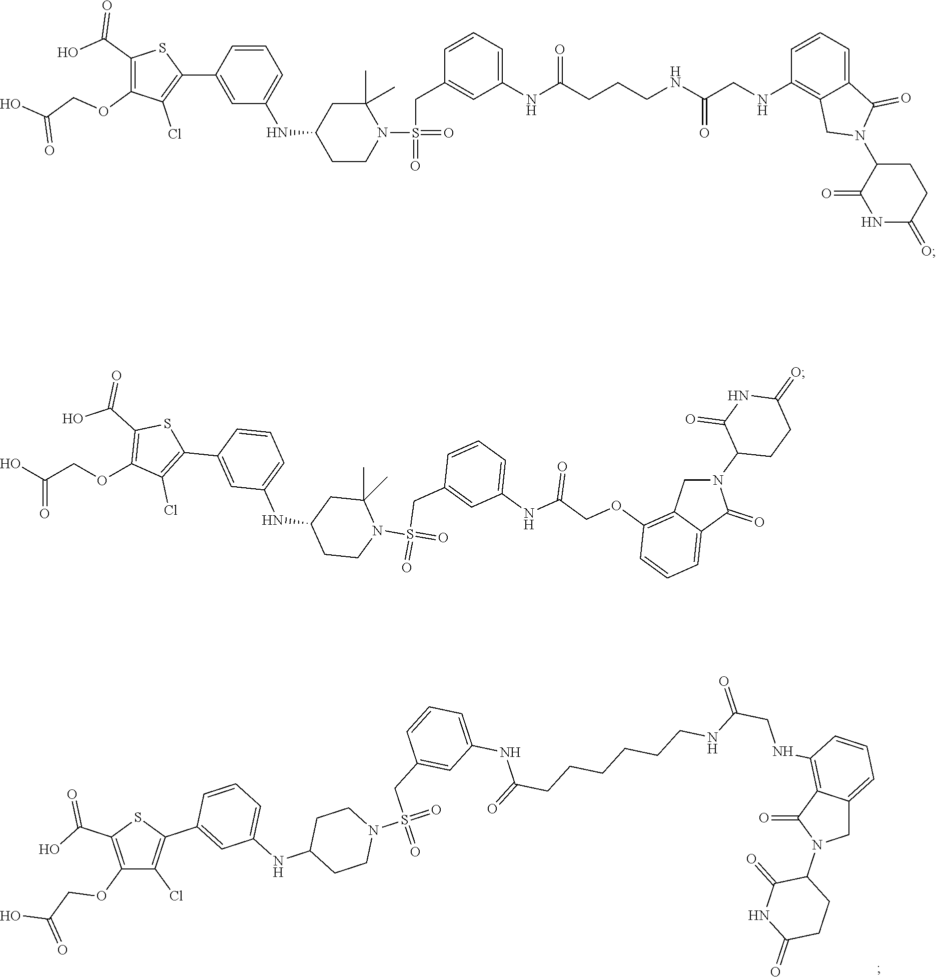 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCCNC(=O)CNc2cccc3c2CN(C2CCC(=O)NC2=O)C3=O)c1 ; CC1(C)C[C@@H](Nc2cccc(-c3sc(C(=O)O)c(OCC(=O)O)c3Cl)c2)CCN1S(=O)(=O)Cc1cccc(NC(=O)COc2cccc3c2CN(C2CCC(=O)NC2=O)C3=O)c1 ; O=C(O)COc1c(C(=O)O)sc(-c2cccc(NC3CCN(S(=O)(=O)Cc4cccc(NC(=O)CCCCCCNC(=O)CNc5cccc6c5C(=O)N(C5CCC(=O)NC5=O)C6)c4)CC3)c2)c1Cl] 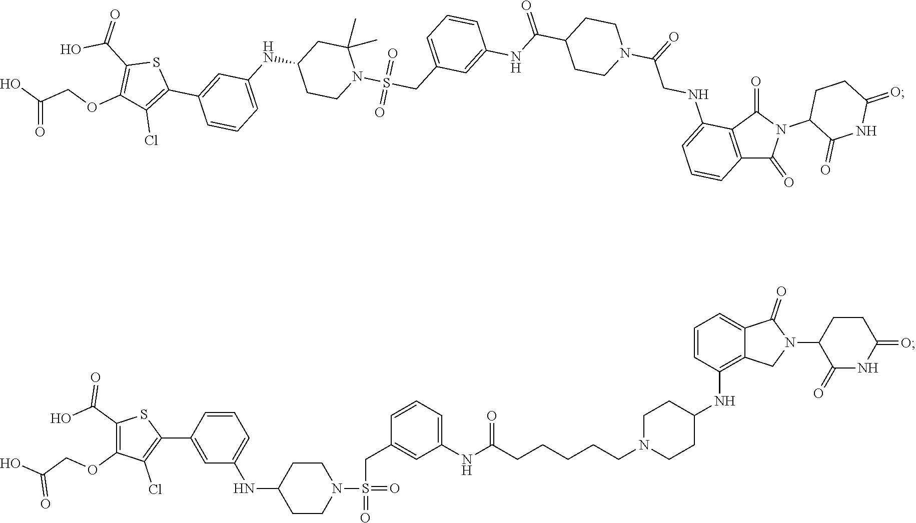 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3cccc4c3C(=O)N(C3CCC(=O)NC3=O)C4=O)CC2)c1 ; O=C(O)COc1c(C(=O)O)sc(-c2cccc(NC3CCN(S(=O)(=O)Cc4cccc(NC(=O)CCCCCN5CCC(Nc6cccc7c6CN(C6CCC(=O)NC6=O)C7=O)CC5)c4)CC3)c2)c1Cl] 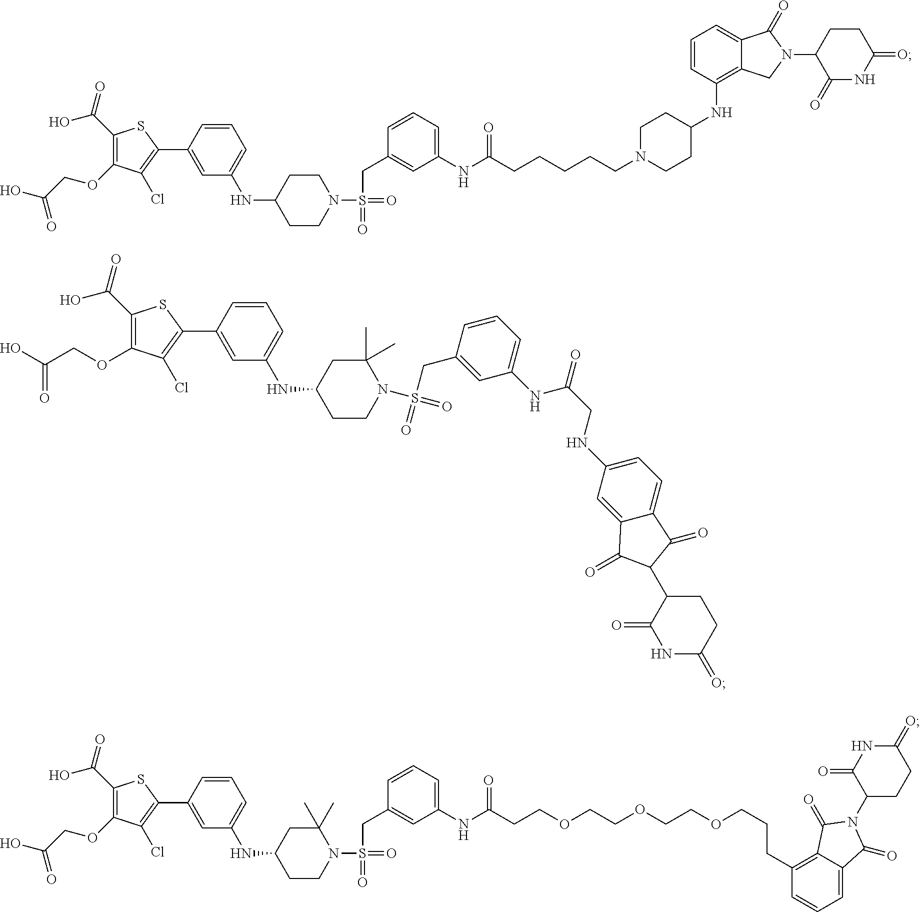 [SMILES: O=C(O)COc1c(C(=O)O)sc(-c2cccc(NC3CCN(S(=O)(=O)Cc4cccc(NC(=O)CCCCCN5CCC(Nc6cccc7c6CN(C6CCC(=O)NC6=O)C7=O)CC5)c4)CC3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)CNc2ccc3c(c2)C(=O)C(C2CCC(=O)NC2=O)C3=O)c1 ; CC1(C)C[C@@H](Nc2cccc(-c3sc(C(=O)O)c(OCC(=O)O)c3Cl)c2)CCN1S(=O)(=O)Cc1cccc(NC(=O)CCOCCOCCOCCCc2cccc3c2C(=O)N(C2CCC(=O)NC2=O)C3=O)c1] 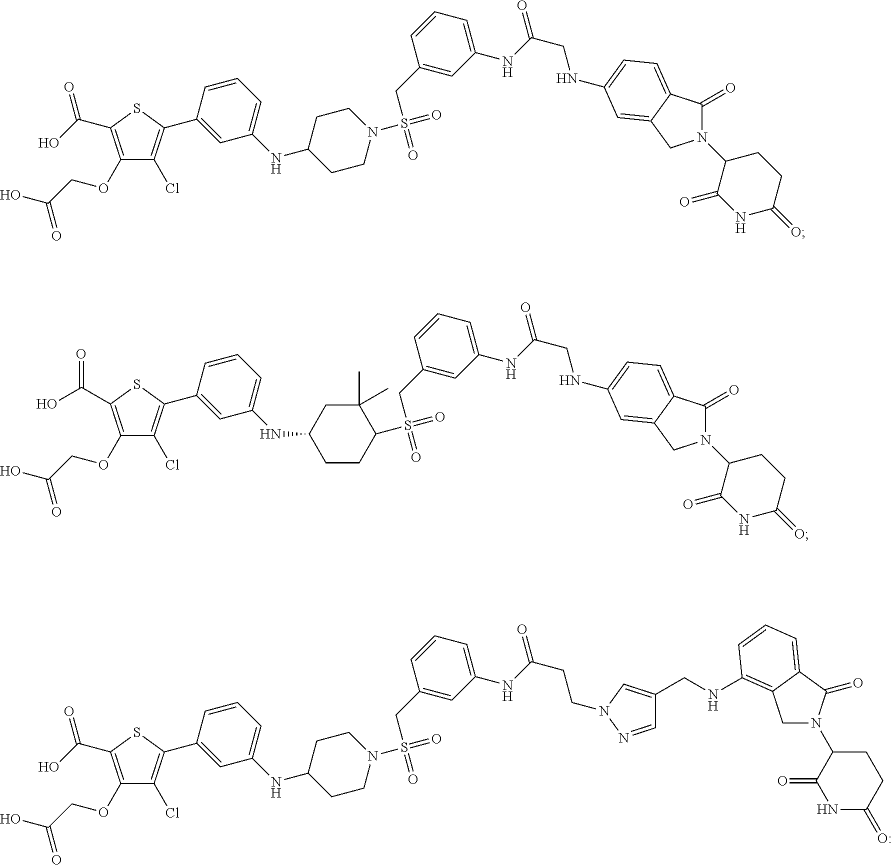 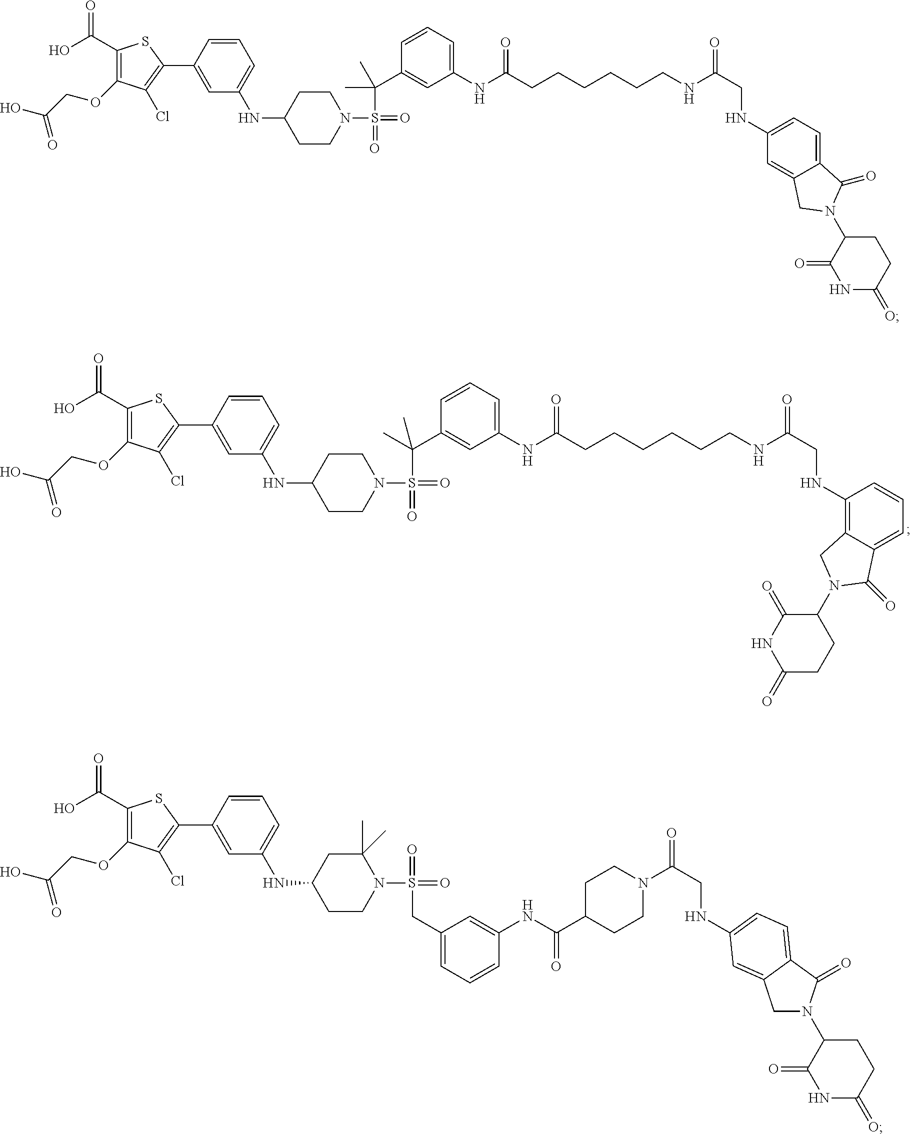 [SMILES: CC(C)(c1cccc(NC(=O)CCCCCCNC(=O)CNc2ccc3c(c2)CN(C2CCC(=O)NC2=O)C3=O)c1)S(=O)(=O)N1CCC(Nc2cccc(-c3sc(C(=O)O)c(OCC(=O)O)c3Cl)c2)CC1 ; CC(C)(c1cccc(NC(=O)CCCCCCNC(=O)CNc2cccc3c2CN(C2CCC(=O)NC2=O)C3=O)c1)S(=O)(=O)N1CCC(Nc2cccc(-c3sc(C(=O)O)c(OCC(=O)O)c3Cl)c2)CC1 ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3ccc4c(c3)CN(C3CCC(=O)NC3=O)C4=O)CC2)c1] 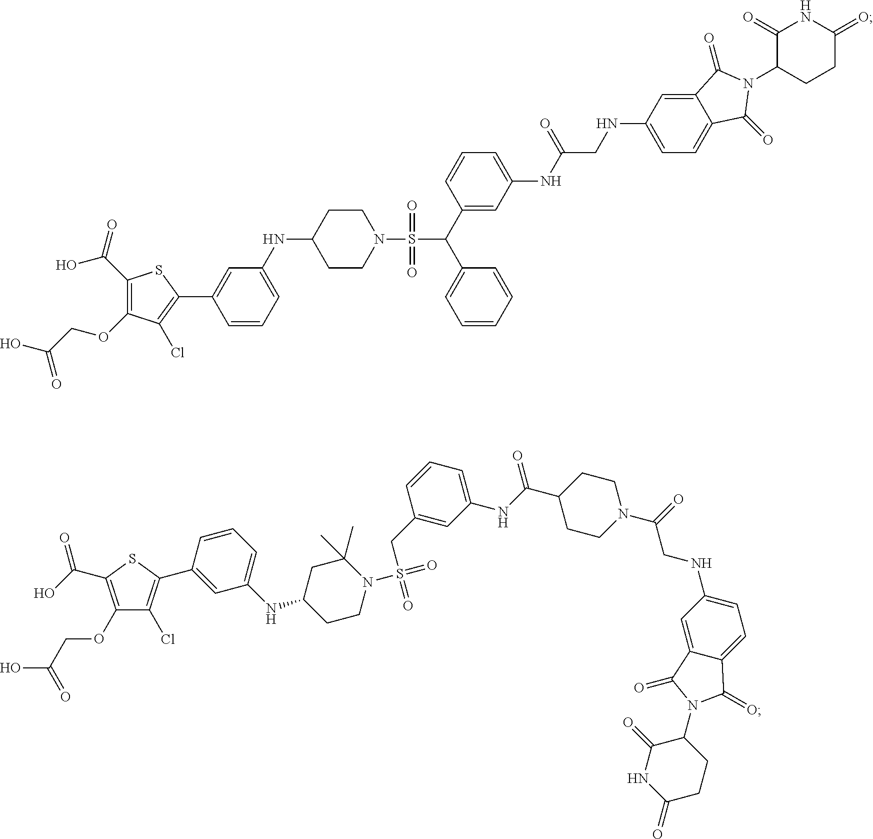 [SMILES: O=C(O)COc1c(C(=O)O)sc(-c2cccc(NC3CCN(S(=O)(=O)C(c4ccccc4)c4cccc(NC(=O)CNc5ccc6c(c5)C(=O)N(C5CCC(=O)NC5=O)C6=O)c4)CC3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3ccc4c(c3)C(=O)N(C3CCC(=O)NC3=O)C4=O)CC2)c1] 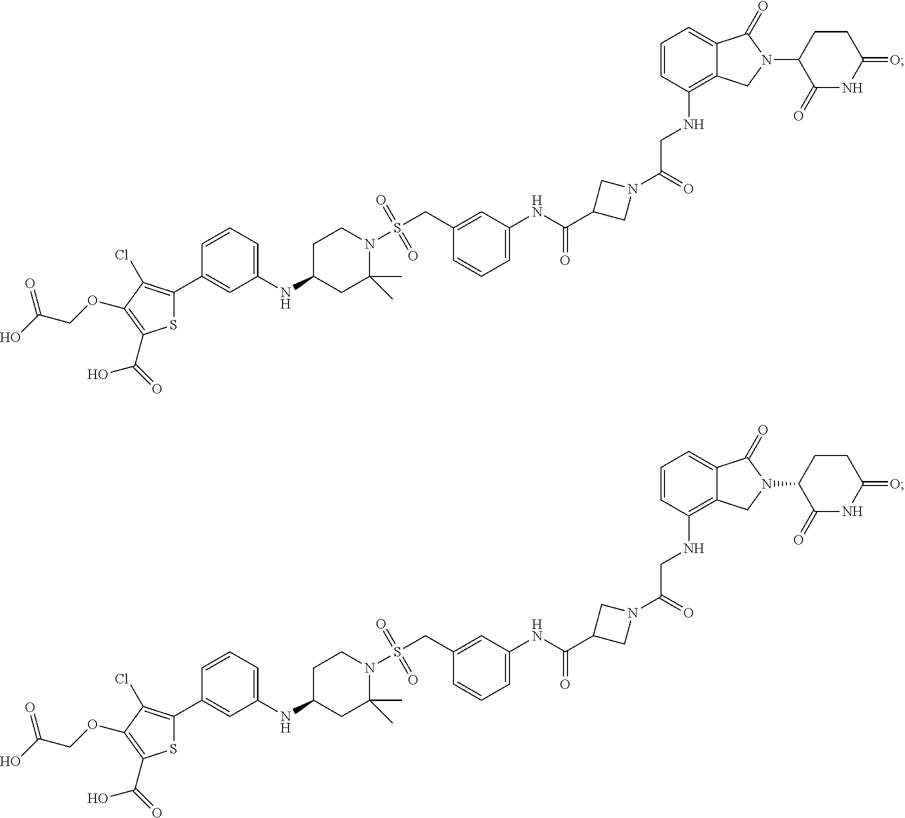 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3cccc4c3CN(C3CCC(=O)NC3=O)C4=O)C2)c1 ; CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3cccc4c3CN([C@@H]3CCC(=O)NC3=O)C4=O)C2)c1] 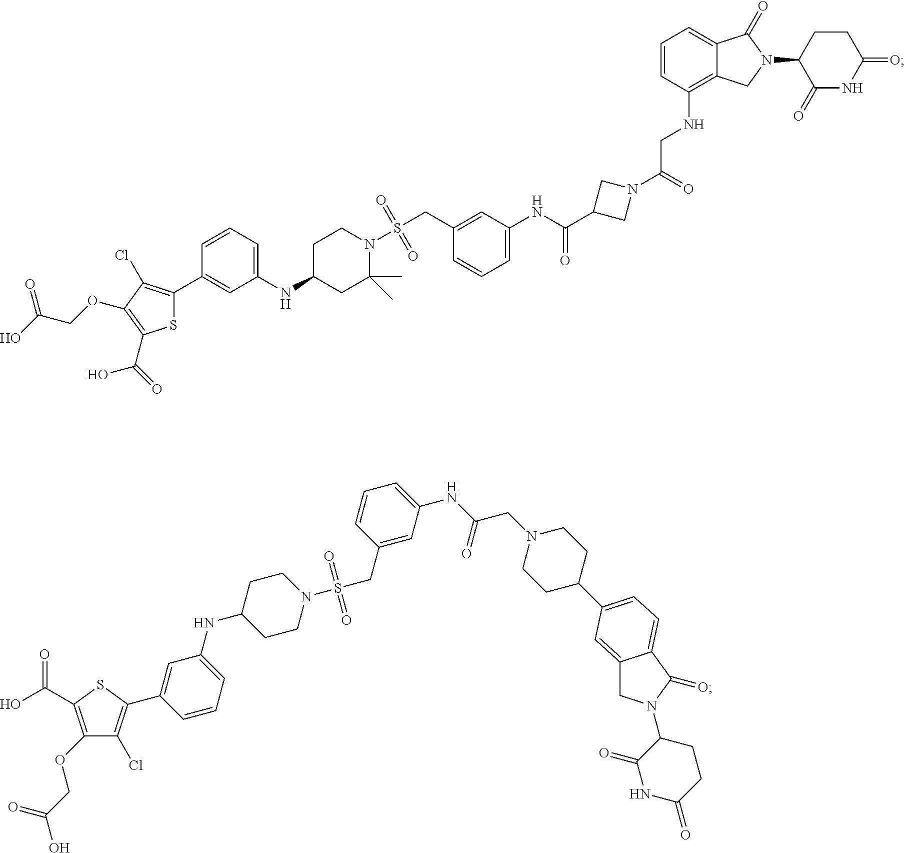 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3cccc4c3CN([C@H]3CCC(=O)NC3=O)C4=O)C2)c1 ; O=C(O)COc1c(C(=O)O)sc(-c2cccc(NC3CCN(S(=O)(=O)Cc4cccc(NC(=O)CN5CCC(c6ccc7c(c6)CN(C6CCC(=O)NC6=O)C7=O)CC5)c4)CC3)c2)c1Cl] 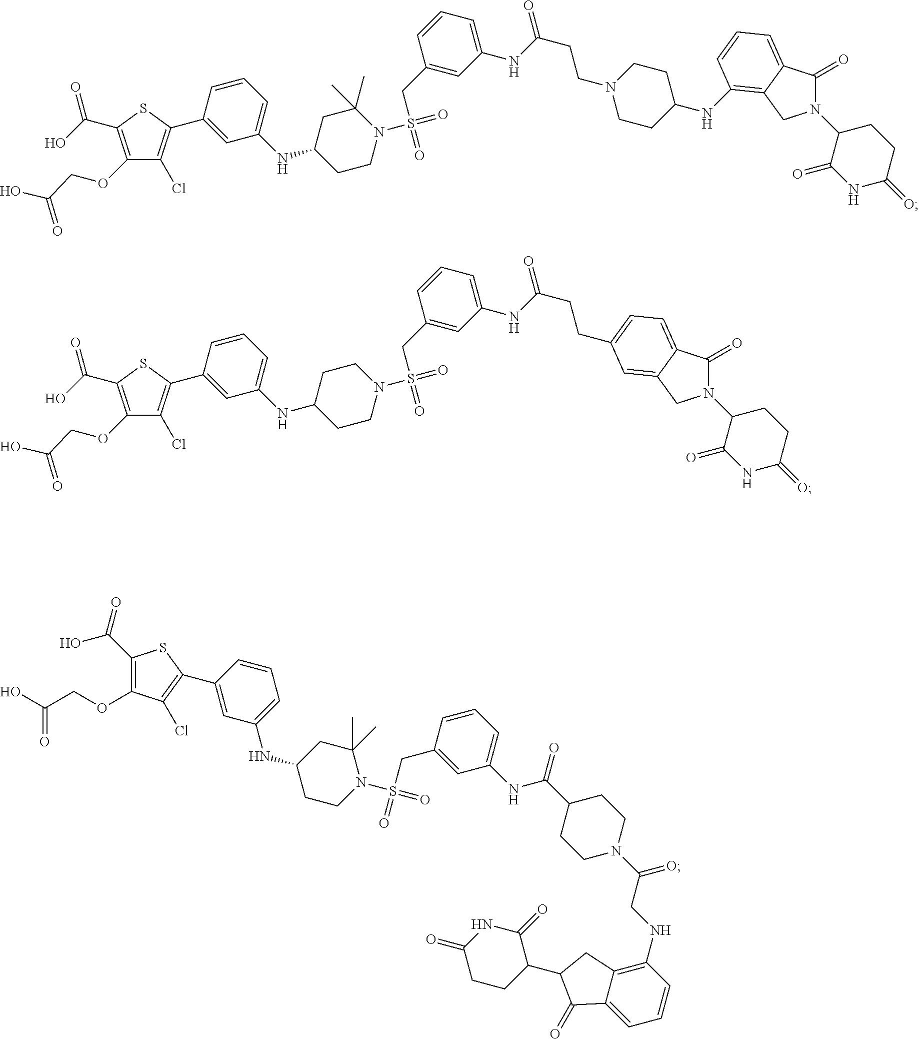 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCN2CCC(Nc3cccc4c3CN(C3CCC(=O)NC3=O)C4=O)CC2)c1 ; O=C(O)COc1c(C(=O)O)sc(-c2cccc(NC3CCN(S(=O)(=O)Cc4cccc(NC(=O)CCc5ccc6c(c5)CN(C5CCC(=O)NC5=O)C6=O)c4)CC3)c2)c1Cl ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CNc3cccc4c3CC(C3CCC(=O)NC3=O)C4=O)CC2)c1] 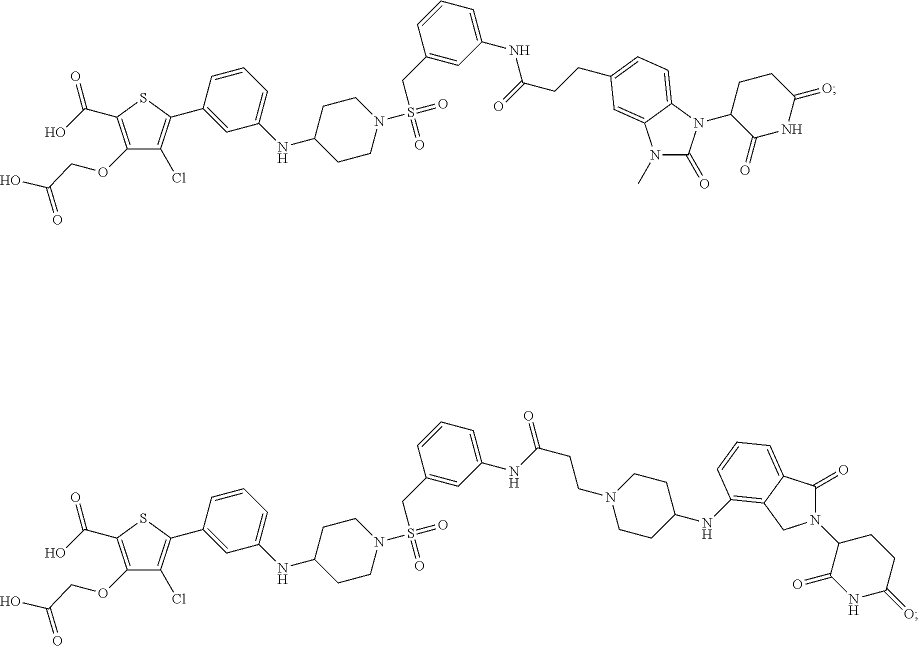 [SMILES: Cn1c(=O)n(C2CCC(=O)NC2=O)c2ccc(CCC(=O)Nc3cccc(CS(=O)(=O)N4CCC(Nc5cccc(-c6sc(C(=O)O)c(OCC(=O)O)c6Cl)c5)CC4)c3)cc21 ; O=C(O)COc1c(C(=O)O)sc(-c2cccc(NC3CCN(S(=O)(=O)Cc4cccc(NC(=O)CCN5CCC(Nc6cccc7c6CN(C6CCC(=O)NC6=O)C7=O)CC5)c4)CC3)c2)c1Cl] 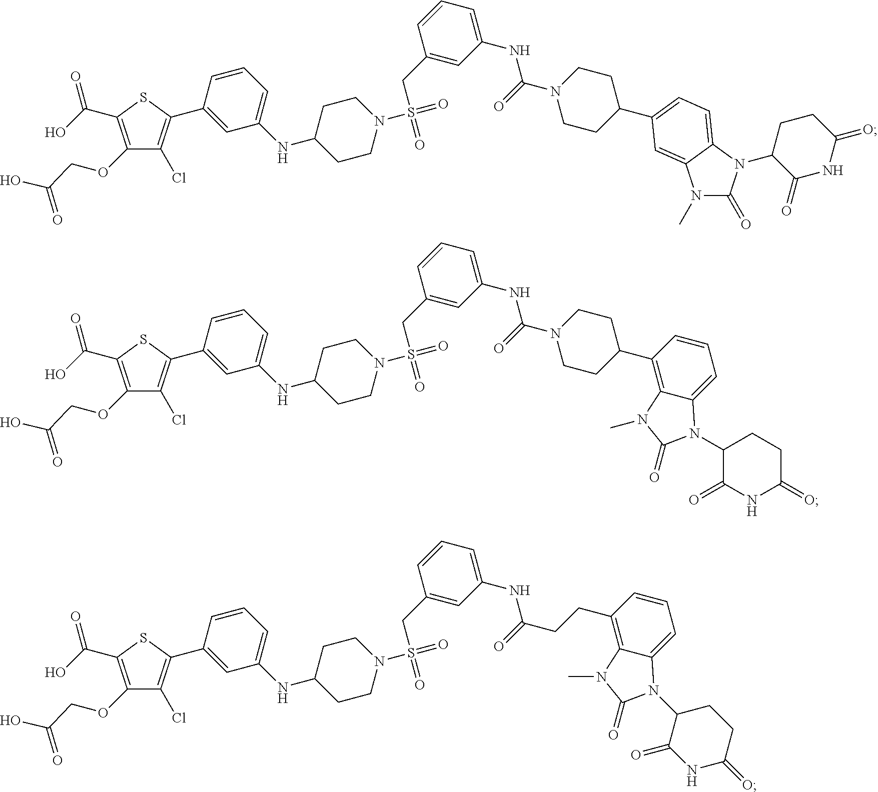 [SMILES: Cn1c(=O)n(C2CCC(=O)NC2=O)c2ccc(C3CCN(C(=O)Nc4cccc(CS(=O)(=O)N5CCC(Nc6cccc(-c7sc(C(=O)O)c(OCC(=O)O)c7Cl)c6)CC5)c4)CC3)cc21 ; Cn1c(=O)n(C2CCC(=O)NC2=O)c2cccc(C3CCN(C(=O)Nc4cccc(CS(=O)(=O)N5CCC(Nc6cccc(-c7sc(C(=O)O)c(OCC(=O)O)c7Cl)c6)CC5)c4)CC3)c21 ; Cn1c(=O)n(C2CCC(=O)NC2=O)c2cccc(CCC(=O)Nc3cccc(CS(=O)(=O)N4CCC(Nc5cccc(-c6sc(C(=O)O)c(OCC(=O)O)c6Cl)c5)CC4)c3)c21] 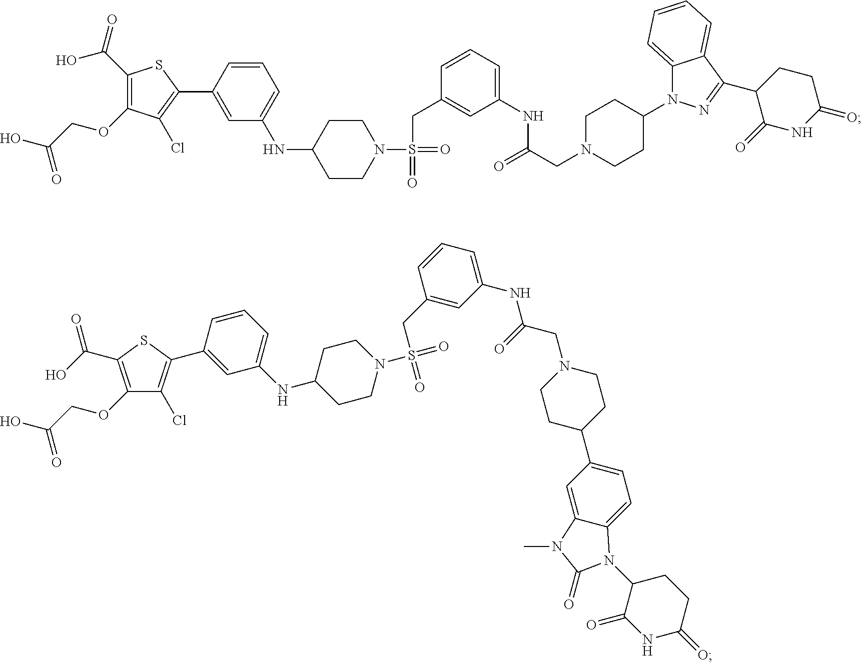 [SMILES: O=C(O)COc1c(C(=O)O)sc(-c2cccc(NC3CCN(S(=O)(=O)Cc4cccc(NC(=O)CN5CCC(n6nc(C7CCC(=O)NC7=O)c7ccccc76)CC5)c4)CC3)c2)c1Cl ; Cn1c(=O)n(C2CCC(=O)NC2=O)c2ccc(C3CCN(CC(=O)Nc4cccc(CS(=O)(=O)N5CCC(Nc6cccc(-c7sc(C(=O)O)c(OCC(=O)O)c7Cl)c6)CC5)c4)CC3)cc21] 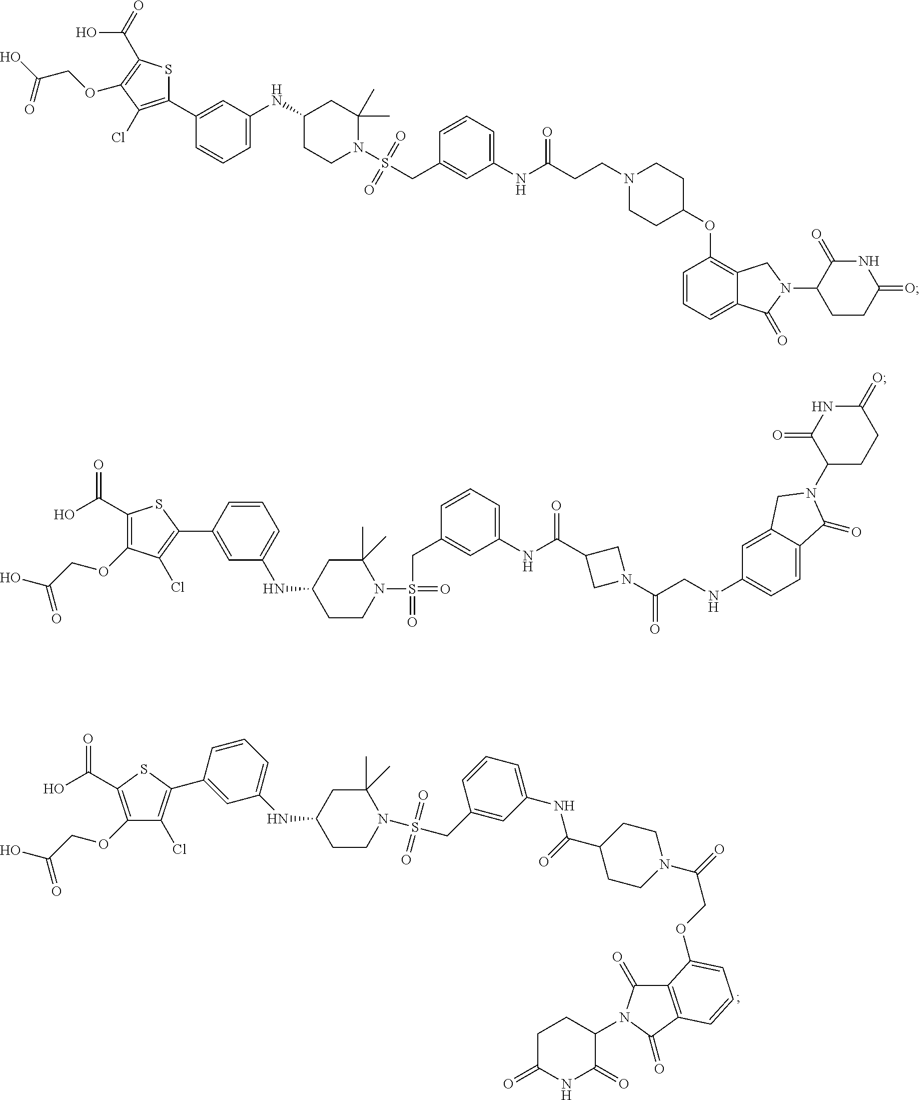 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCN2CCC(Oc3cccc4c3CN(C3CCC(=O)NC3=O)C4=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)C2CN(C(=O)CNc3ccc4c(c3)CN(C3CCC(=O)NC3=O)C4=O)C2)c1 ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)COc3cccc4c3C(=O)N(C3CCC(=O)NC3=O)C4=O)CC2)c1] 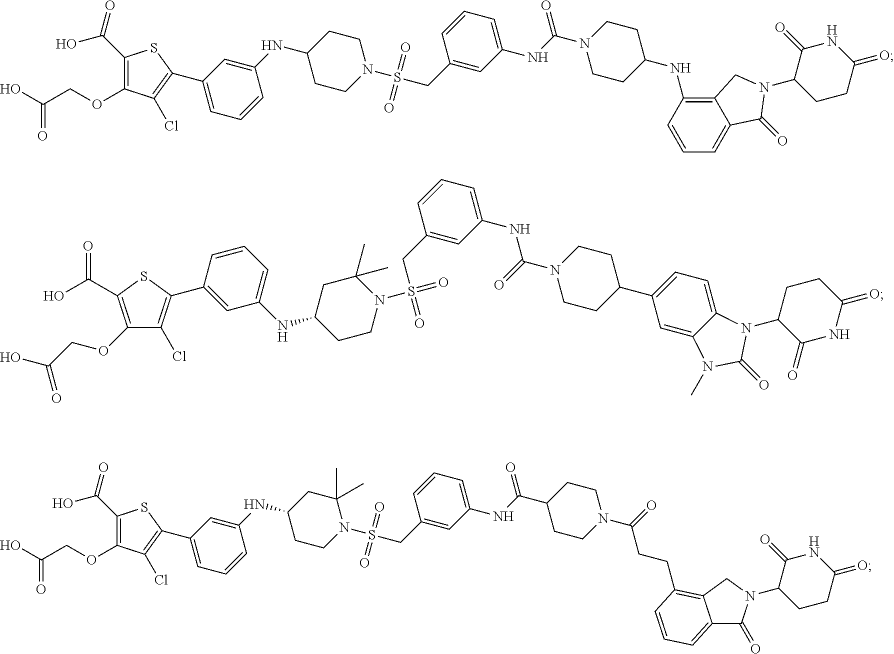 [SMILES: O=C(O)COc1c(C(=O)O)sc(-c2cccc(NC3CCN(S(=O)(=O)Cc4cccc(NC(=O)N5CCC(Nc6cccc7c6CN(C6CCC(=O)NC6=O)C7=O)CC5)c4)CC3)c2)c1Cl ; Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC1(C)C[C@@H](Nc2cccc(-c3sc(C(=O)O)c(OCC(=O)O)c3Cl)c2)CCN1S(=O)(=O)Cc1cccc(NC(=O)C2CCN(C(=O)CCc3cccc4c3CN(C3CCC(=O)NC3=O)C4=O)CC2)c1] 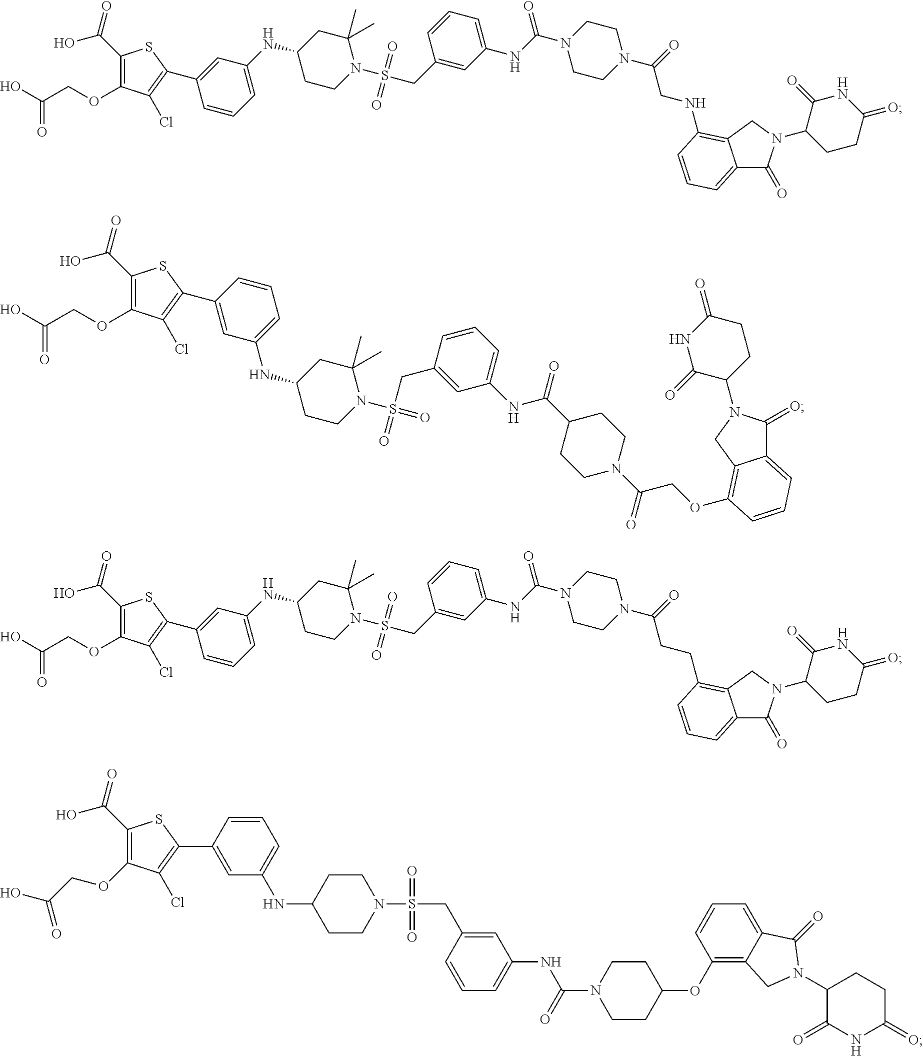 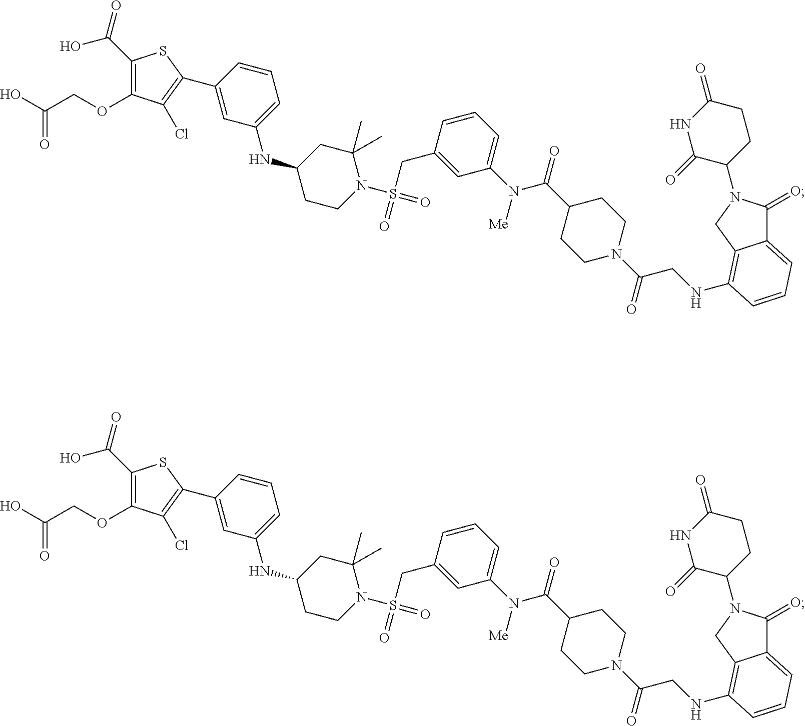 [SMILES: CN(C(=O)C1CCN(C(=O)CNc2cccc3c2CN(C2CCC(=O)NC2=O)C3=O)CC1)c1cccc(CS(=O)(=O)N2CC[C@@H](Nc3cccc(-c4sc(C(=O)O)c(OCC(=O)O)c4Cl)c3)CC2(C)C)c1 ; CN(C(=O)C1CCN(C(=O)CNc2cccc3c2CN(C2CCC(=O)NC2=O)C3=O)CC1)c1cccc(CS(=O)(=O)N2CC[C@H](Nc3cccc(-c4sc(C(=O)O)c(OCC(=O)O)c4Cl)c3)CC2(C)C)c1] 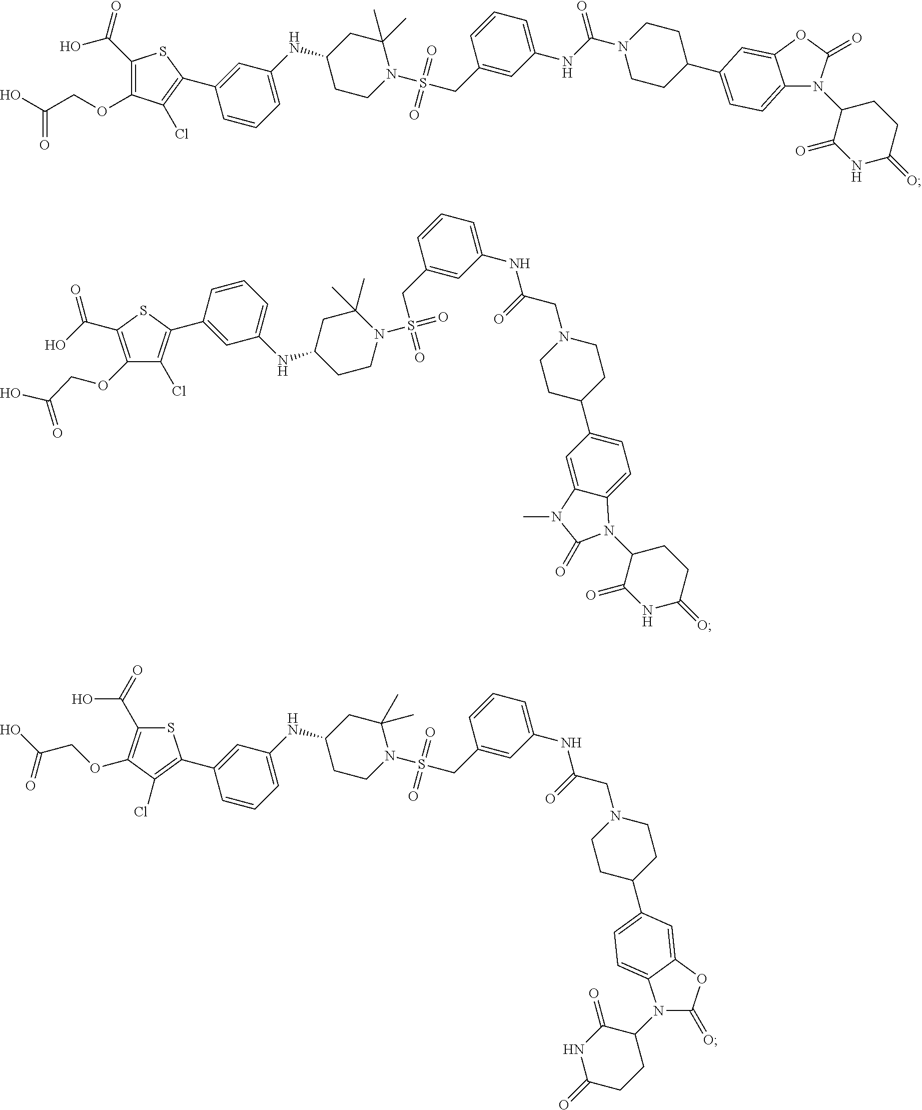 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)oc(=O)n4C3CCC(=O)NC3=O)CC2)c1 ; Cn1c(=O)n(C2CCC(=O)NC2=O)c2ccc(C3CCN(CC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC1(C)C[C@@H](Nc2cccc(-c3sc(C(=O)O)c(OCC(=O)O)c3Cl)c2)CCN1S(=O)(=O)Cc1cccc(NC(=O)CN2CCC(c3ccc4c(c3)oc(=O)n4C3CCC(=O)NC3=O)CC2)c1] 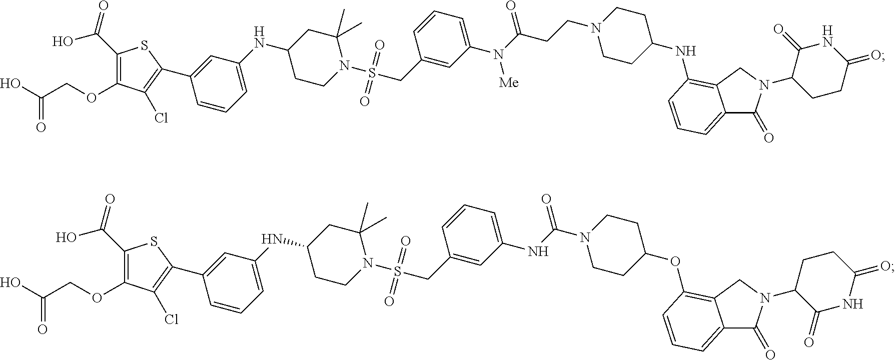 [SMILES: CN(C(=O)CCN1CCC(Nc2cccc3c2CN(C2CCC(=O)NC2=O)C3=O)CC1)c1cccc(CS(=O)(=O)N2CCC(Nc3cccc(-c4sc(C(=O)O)c(OCC(=O)O)c4Cl)c3)CC2(C)C)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(Oc3cccc4c3CN(C3CCC(=O)NC3=O)C4=O)CC2)c1] 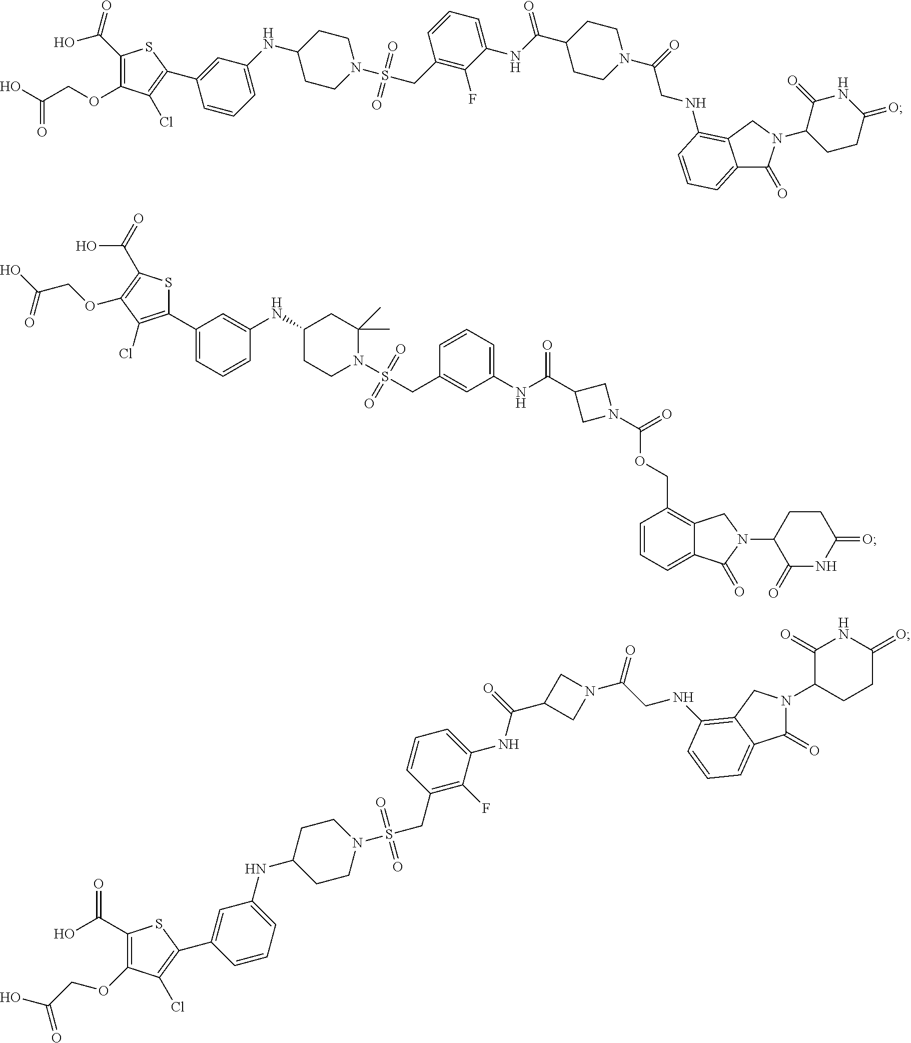 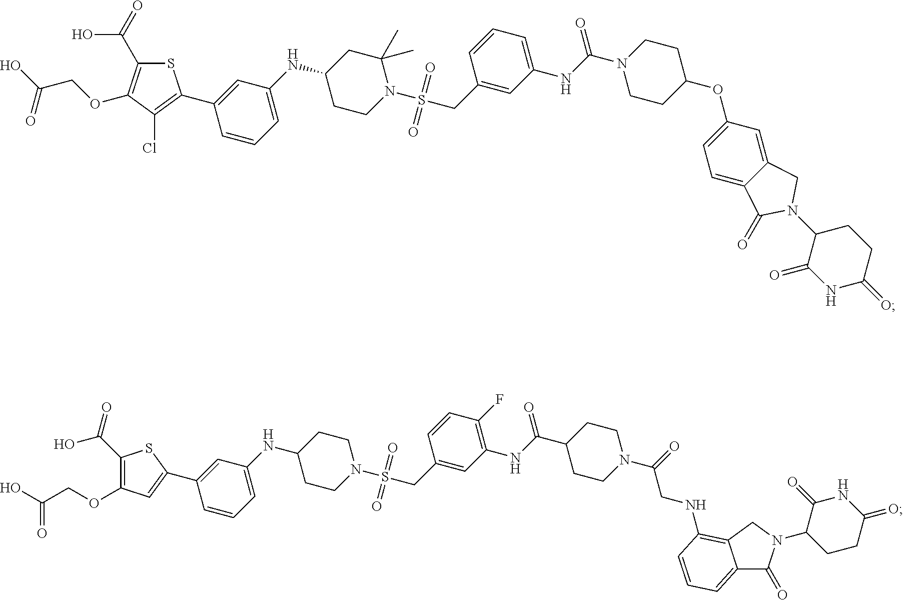 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(Oc3ccc4c(c3)CN(C3CCC(=O)NC3=O)C4=O)CC2)c1 ; O=C(O)COc1cc(-c2cccc(NC3CCN(S(=O)(=O)Cc4ccc(F)c(NC(=O)C5CCN(C(=O)CNc6cccc7c6CN(C6CCC(=O)NC6=O)C7=O)CC5)c4)CC3)c2)sc1C(=O)O] 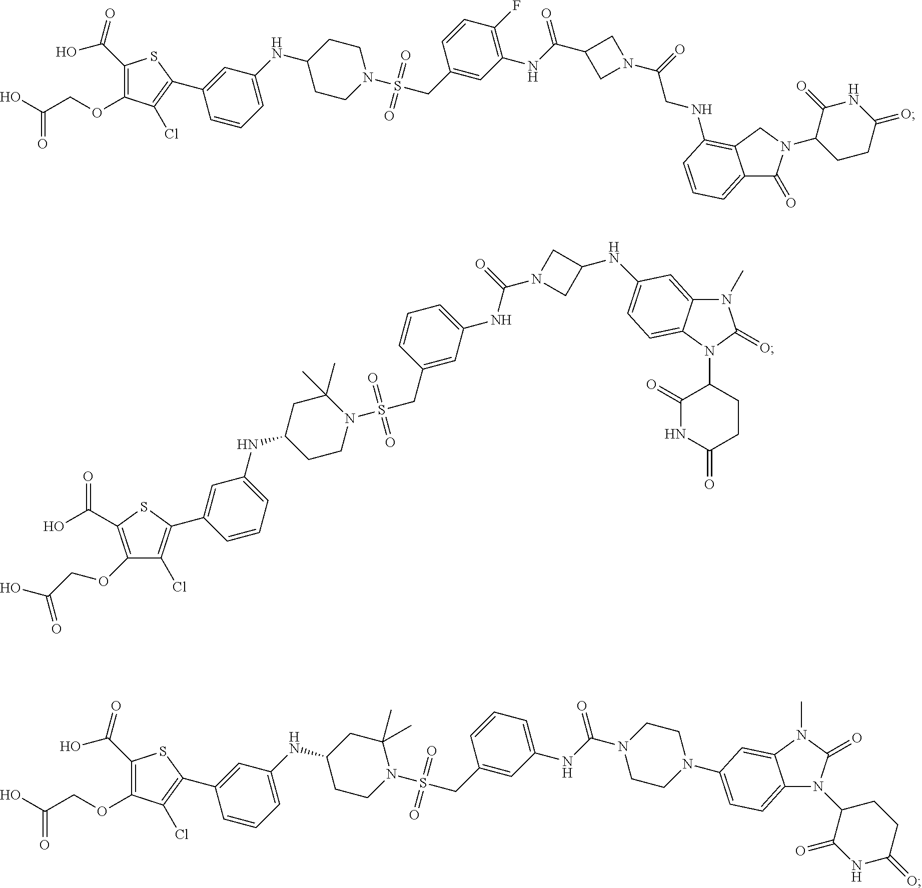 [SMILES: O=C(O)COc1c(C(=O)O)sc(-c2cccc(NC3CCN(S(=O)(=O)Cc4ccc(F)c(NC(=O)C5CN(C(=O)CNc6cccc7c6CN(C6CCC(=O)NC6=O)C7=O)C5)c4)CC3)c2)c1Cl ; Cn1c(=O)n(C2CCC(=O)NC2=O)c2ccc(NC3CN(C(=O)Nc4cccc(CS(=O)(=O)N5CC[C@H](Nc6cccc(-c7sc(C(=O)O)c(OCC(=O)O)c7Cl)c6)CC5(C)C)c4)C3)cc21 ; Cn1c(=O)n(C2CCC(=O)NC2=O)c2ccc(N3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] 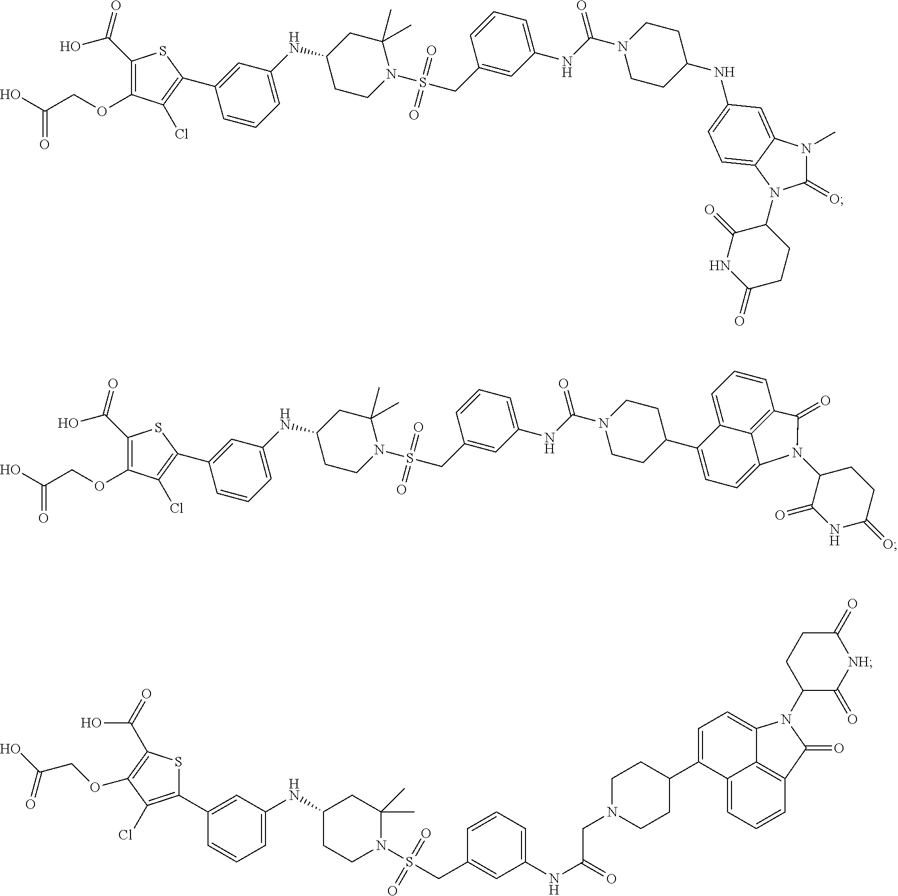 [SMILES: Cn1c(=O)n(C2CCC(=O)NC2=O)c2ccc(NC3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)CN2CCC(c3ccc4c5c(cccc35)C(=O)N4C3CCC(=O)NC3=O)CC2)c1] 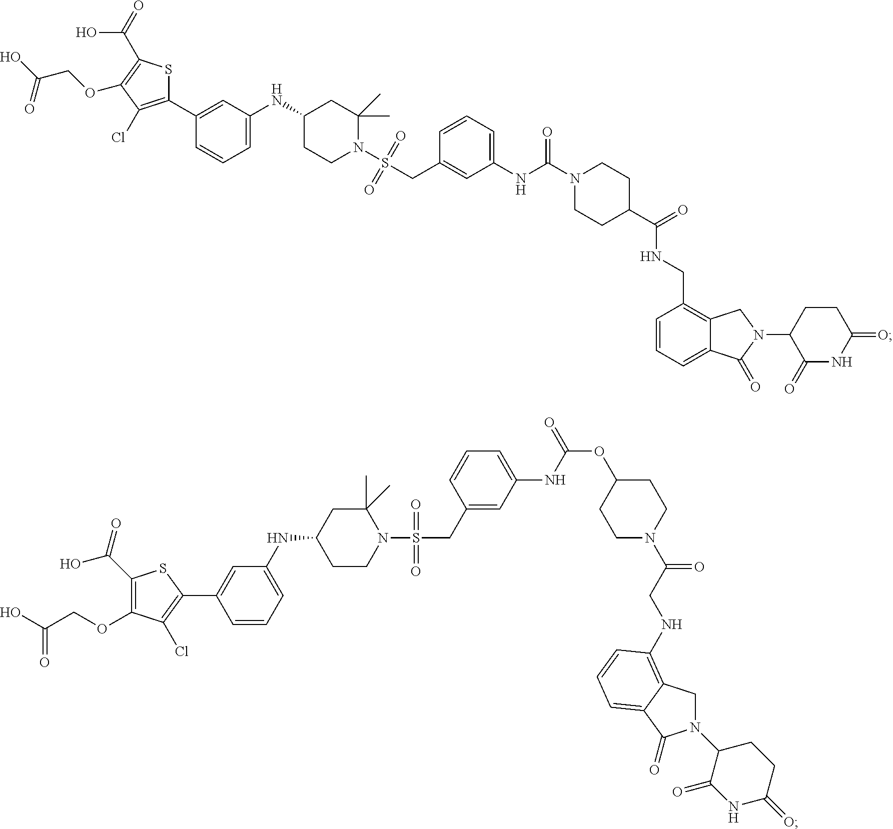 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(C(=O)NCc3cccc4c3CN(C3CCC(=O)NC3=O)C4=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)OC2CCN(C(=O)CNc3cccc4c3CN(C3CCC(=O)NC3=O)C4=O)CC2)c1] 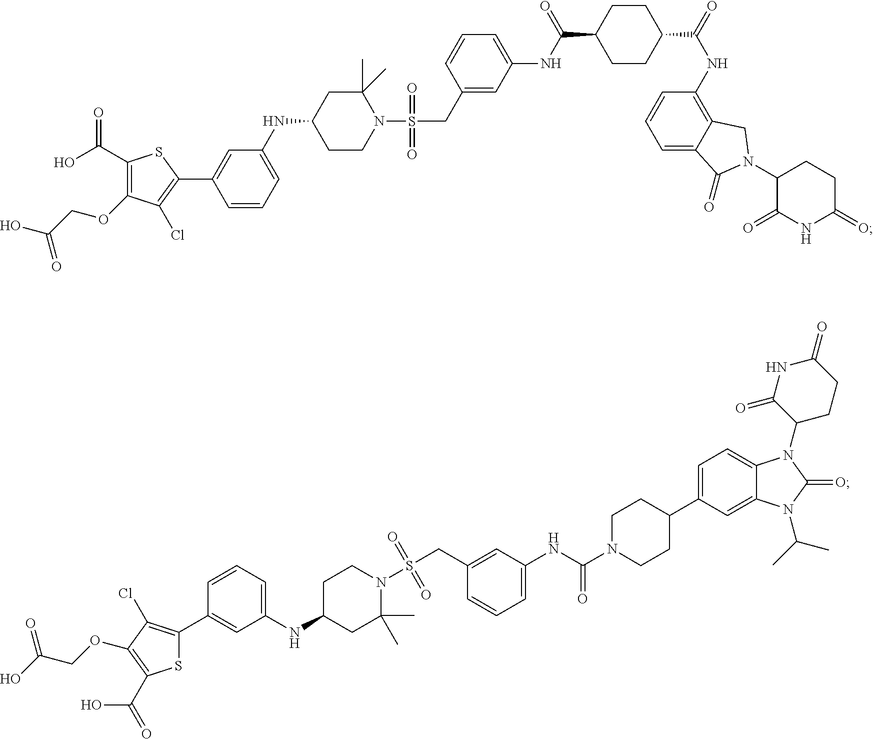 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@H](C(=O)Nc3cccc4c3CN(C3CCC(=O)NC3=O)C4=O)CC2)c1 ; CC(C)n1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] 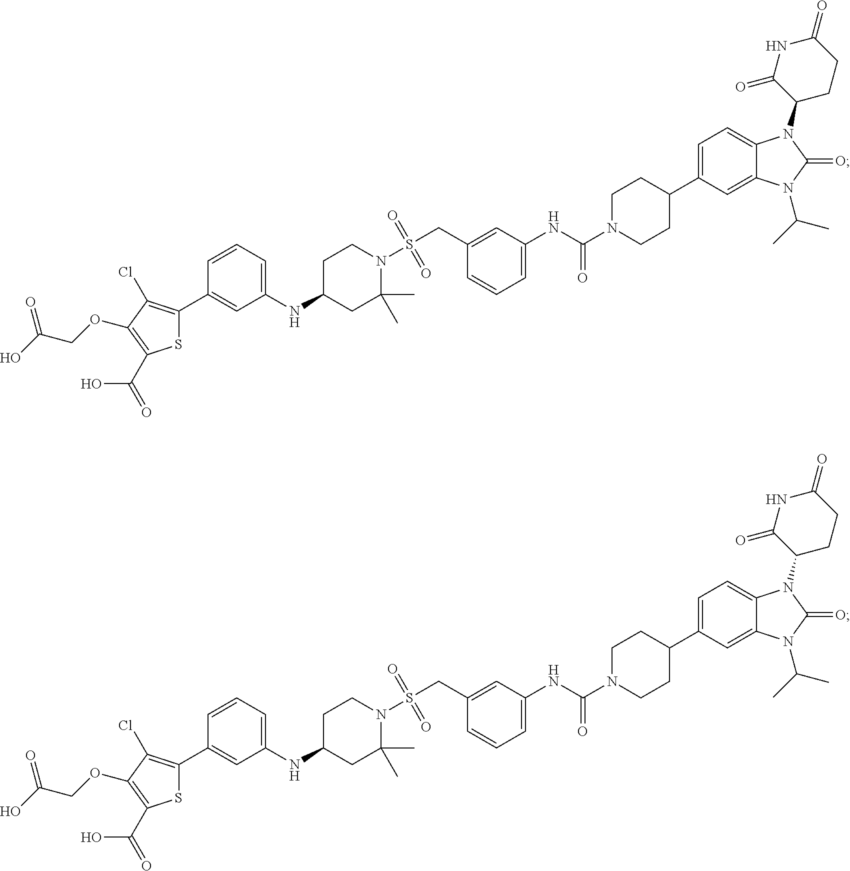 [SMILES: CC(C)n1c(=O)n([C@@H]2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC(C)n1c(=O)n([C@H]2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] 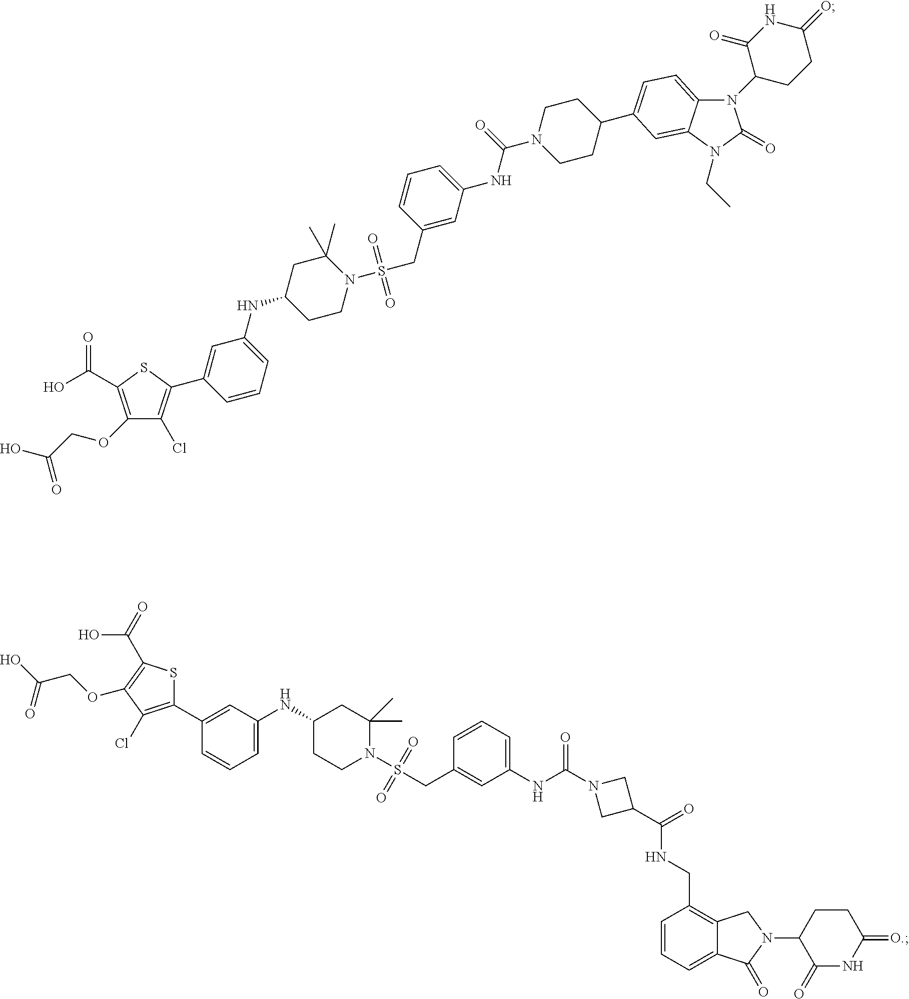 [SMILES: CCn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC1(C)C[C@@H](Nc2cccc(-c3sc(C(=O)O)c(OCC(=O)O)c3Cl)c2)CCN1S(=O)(=O)Cc1cccc(NC(=O)N2CC(C(=O)NCc3cccc4c3CN(C3CCC(=O)NC3=O)C4=O)C2)c1] 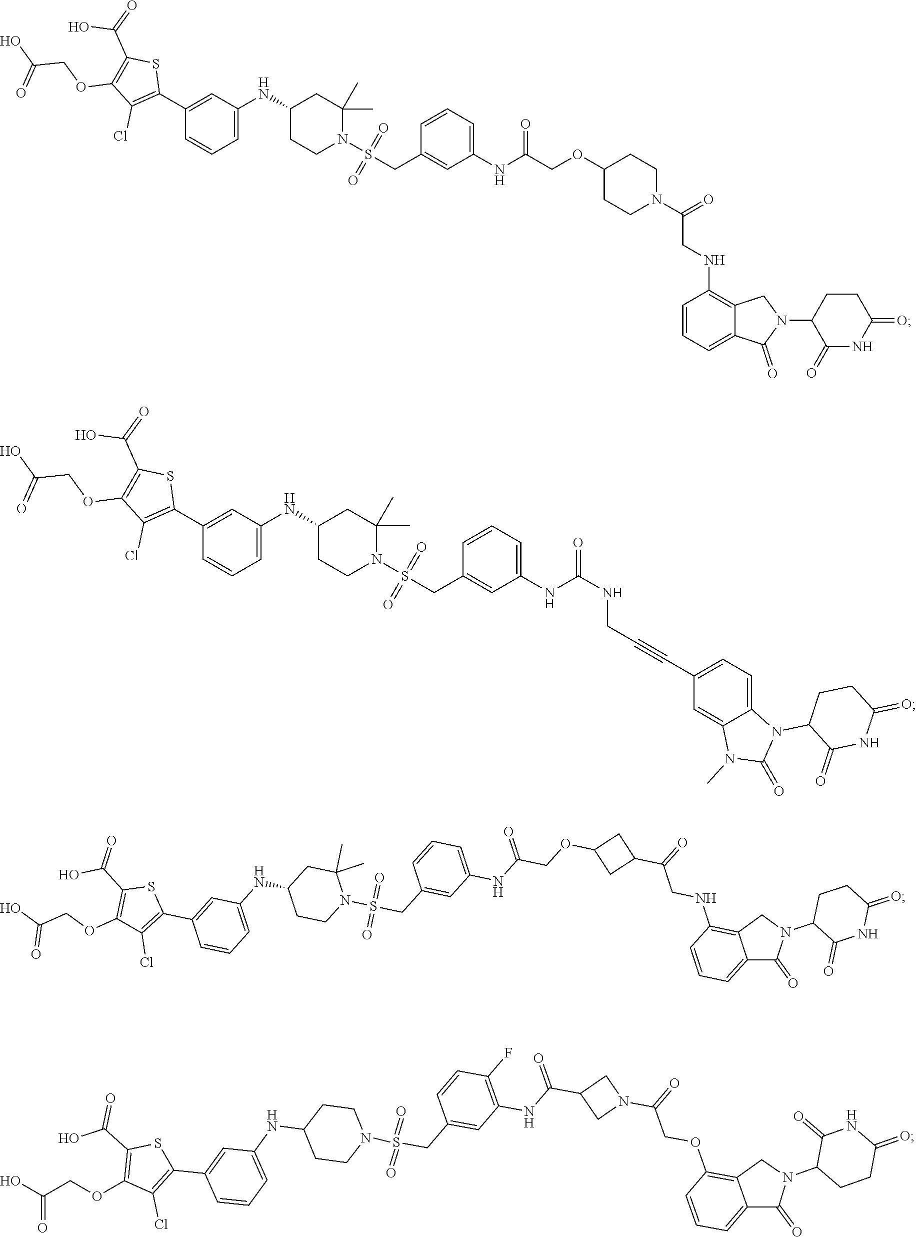 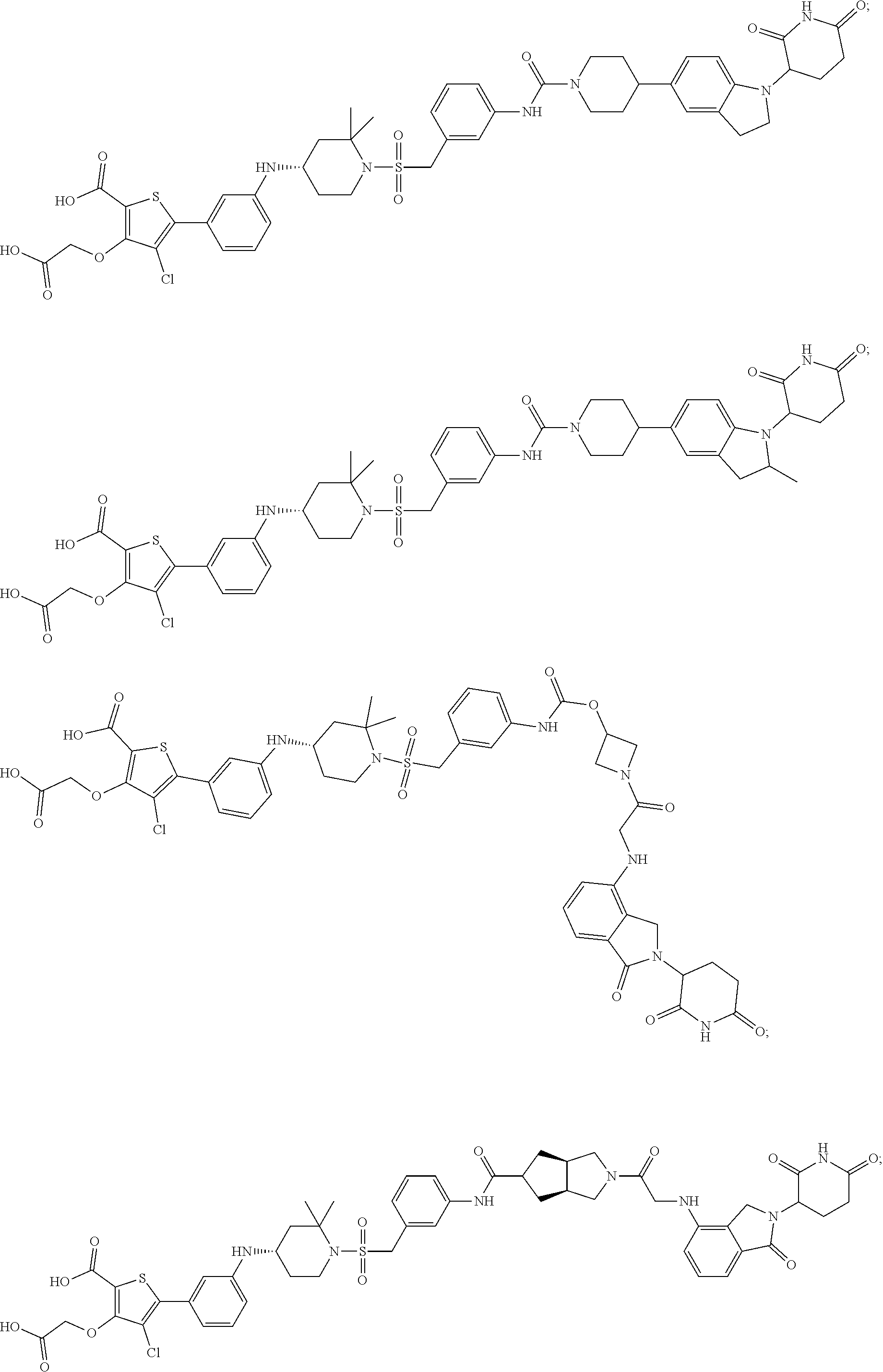 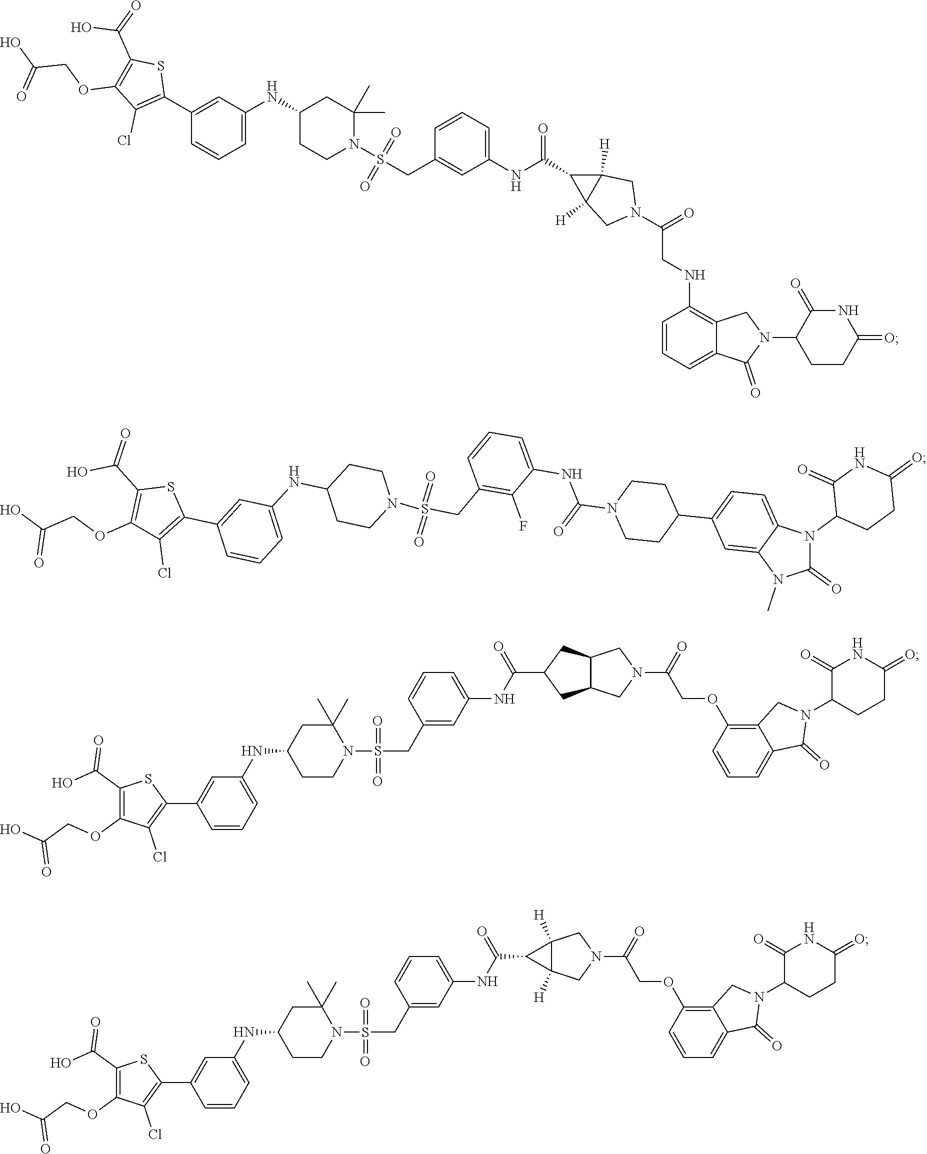 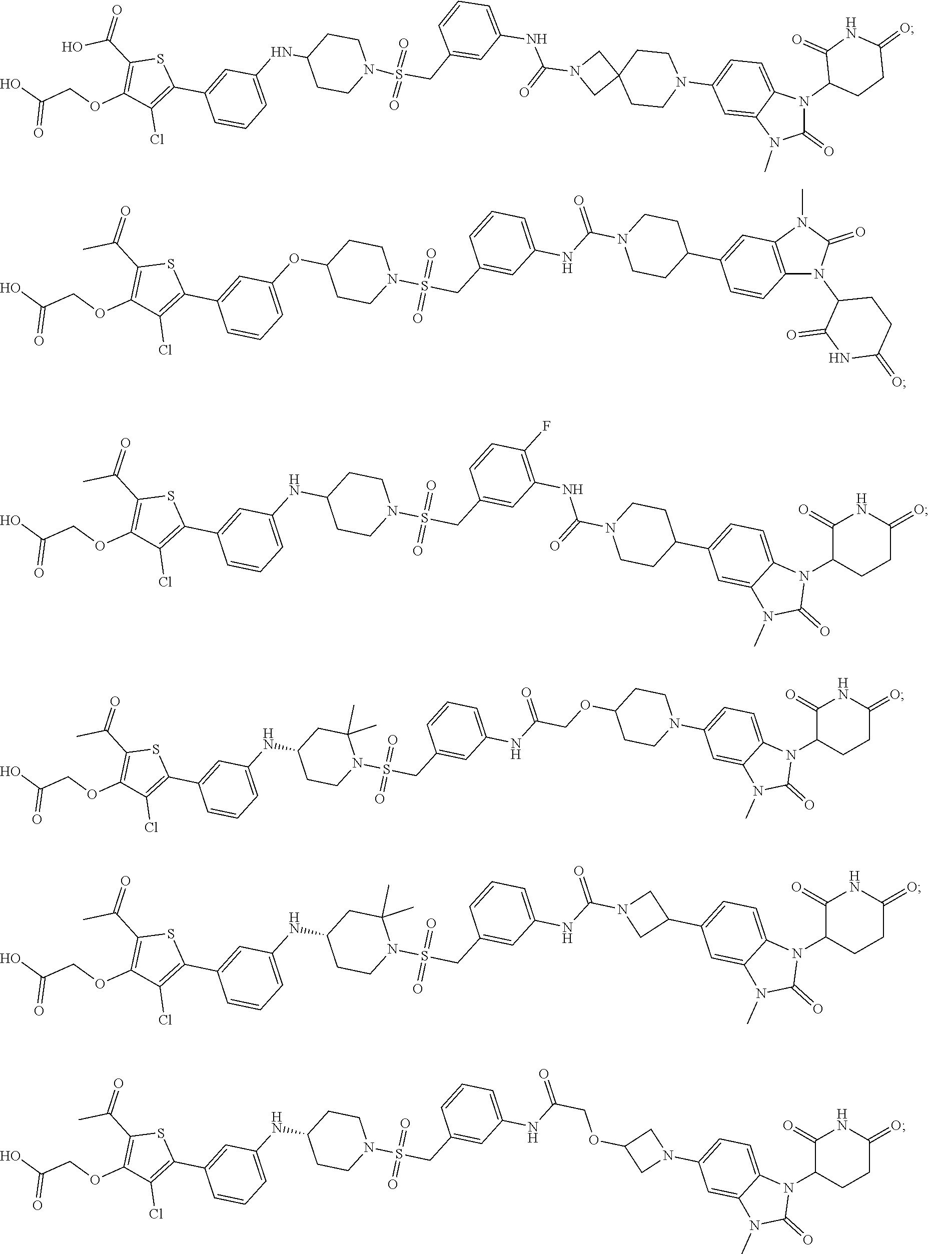 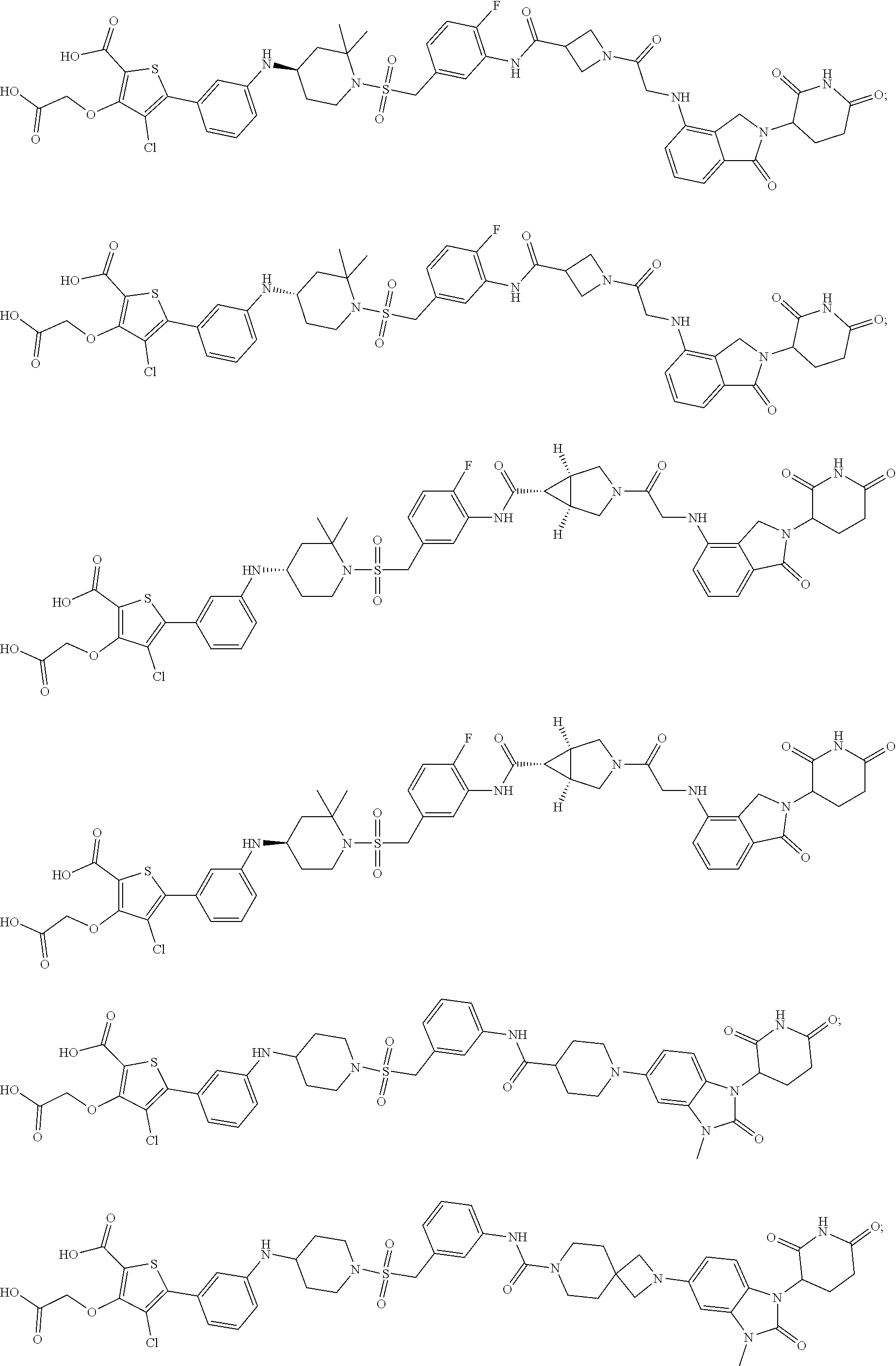 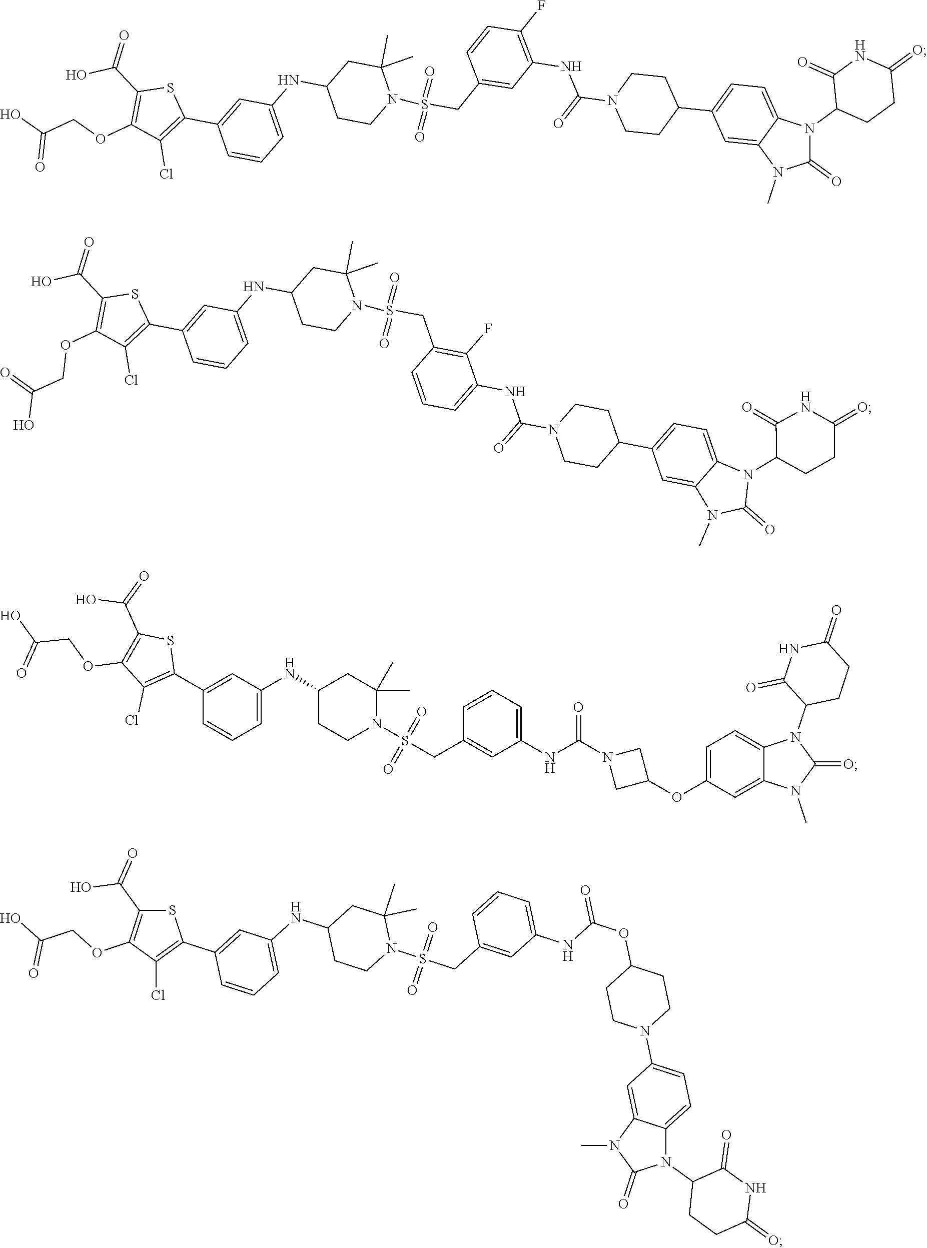 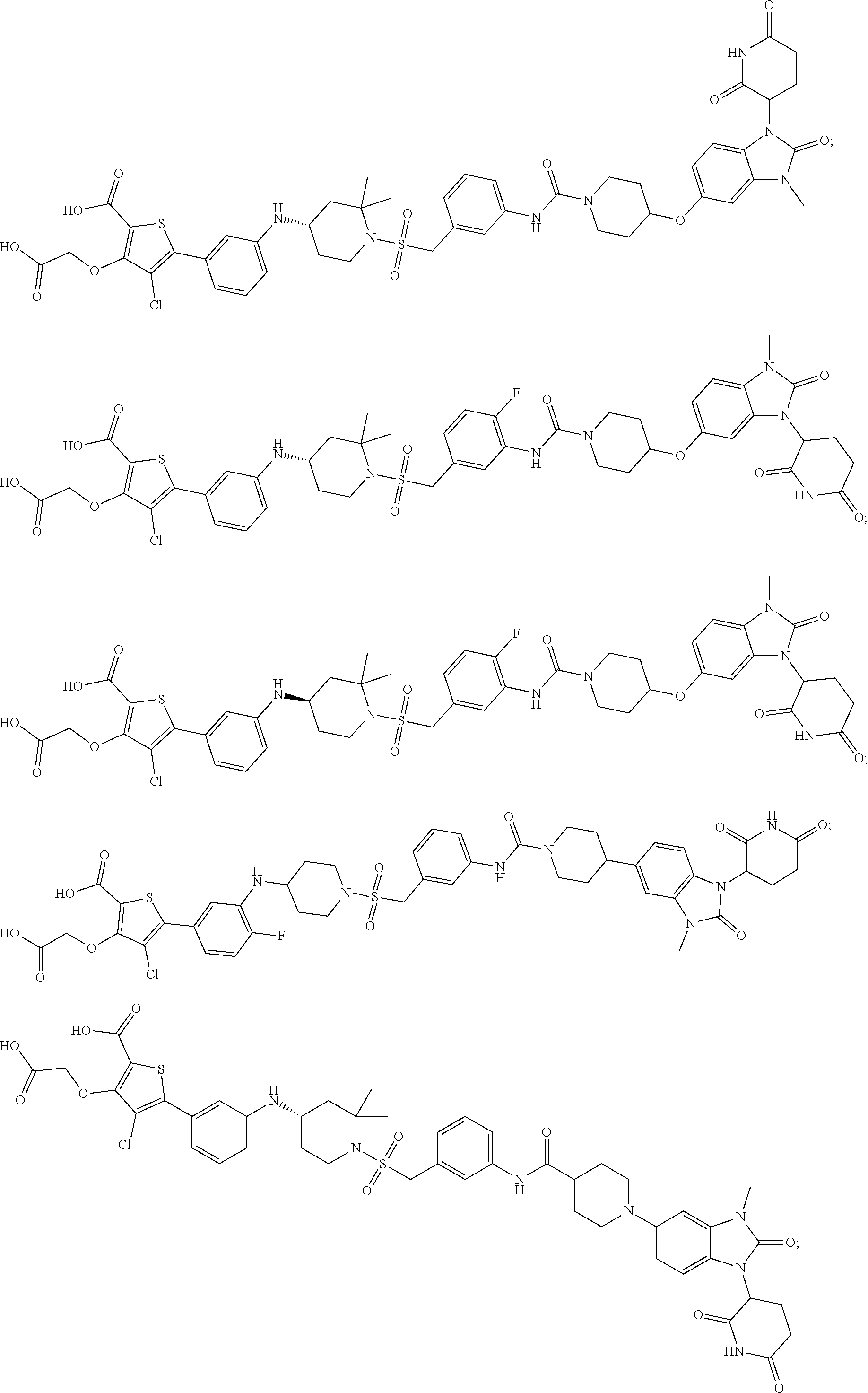 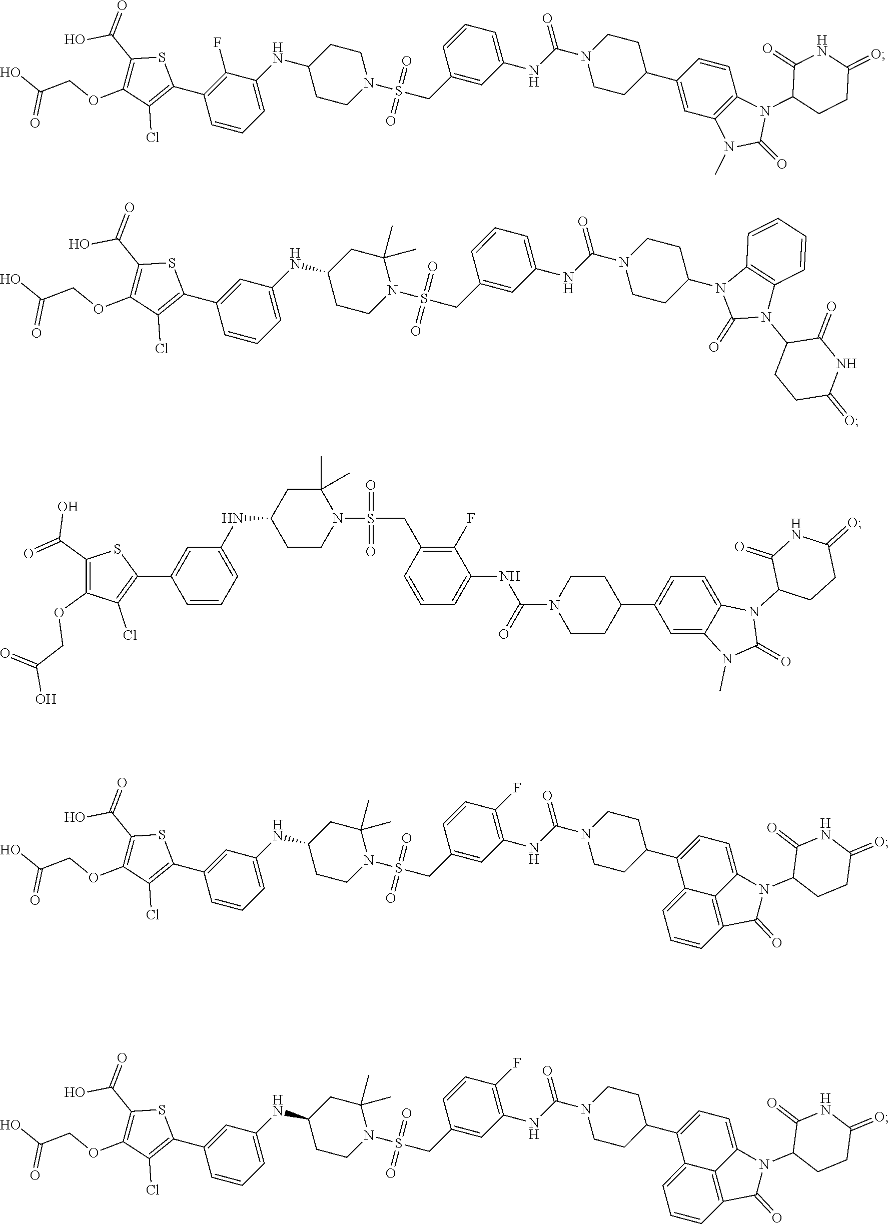 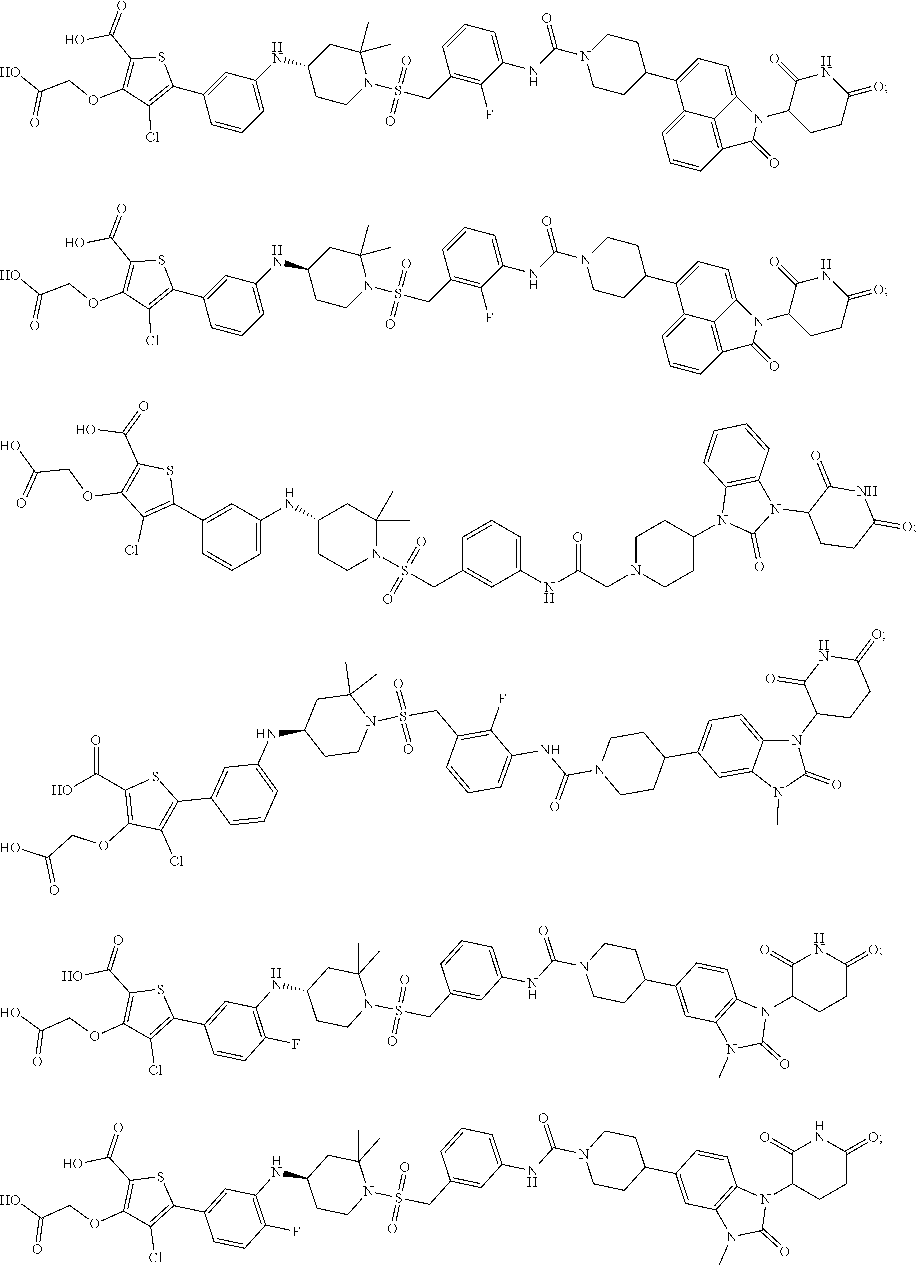 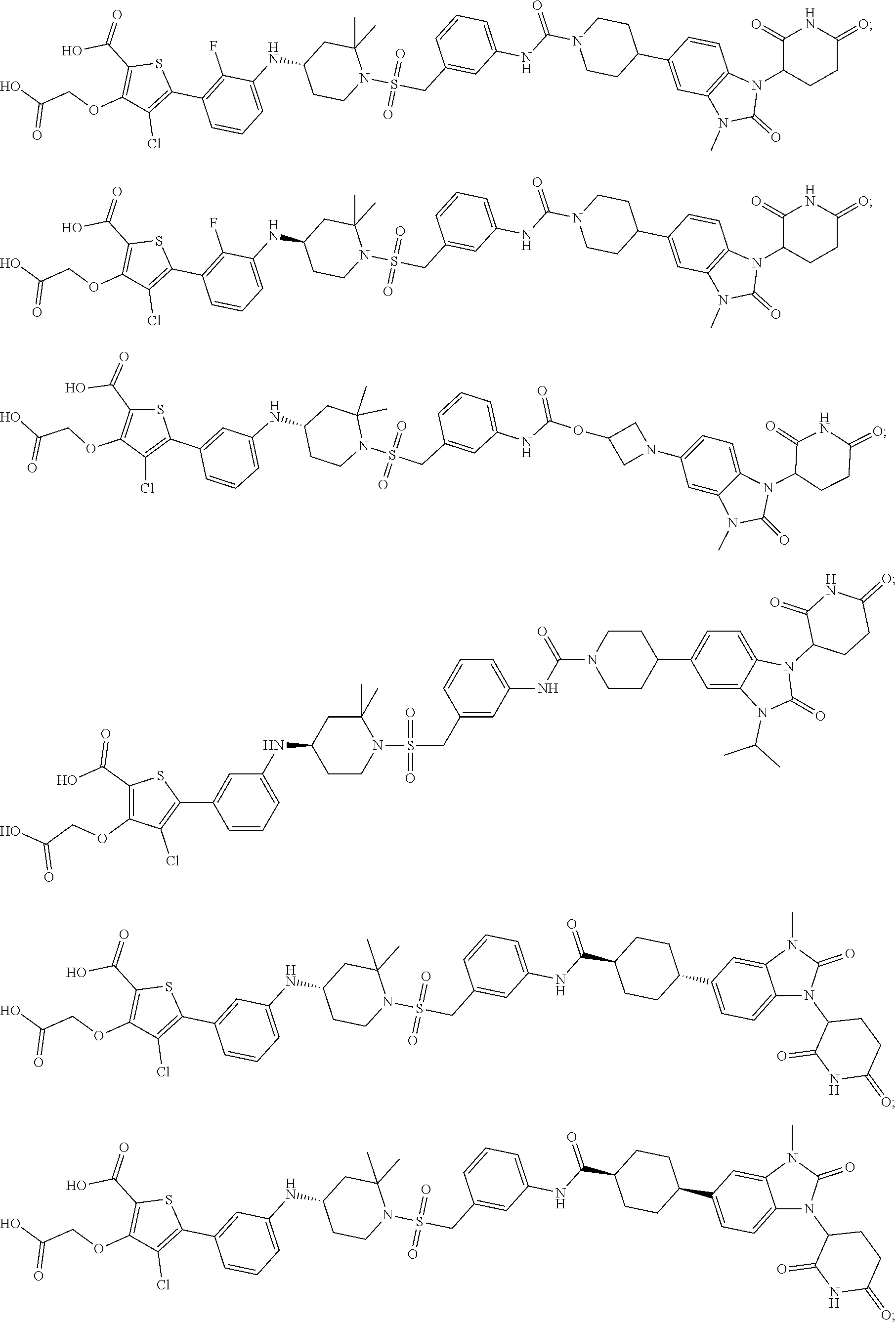 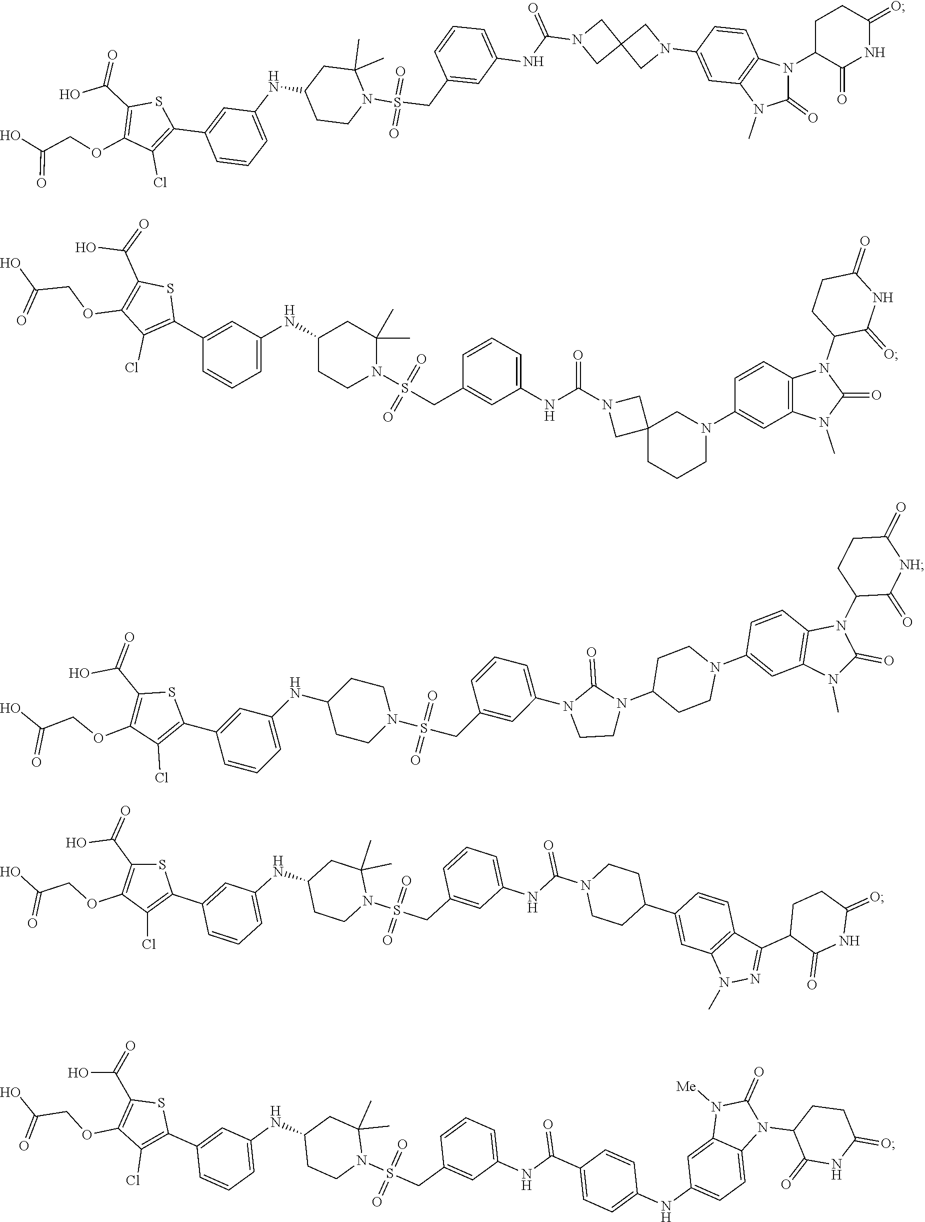 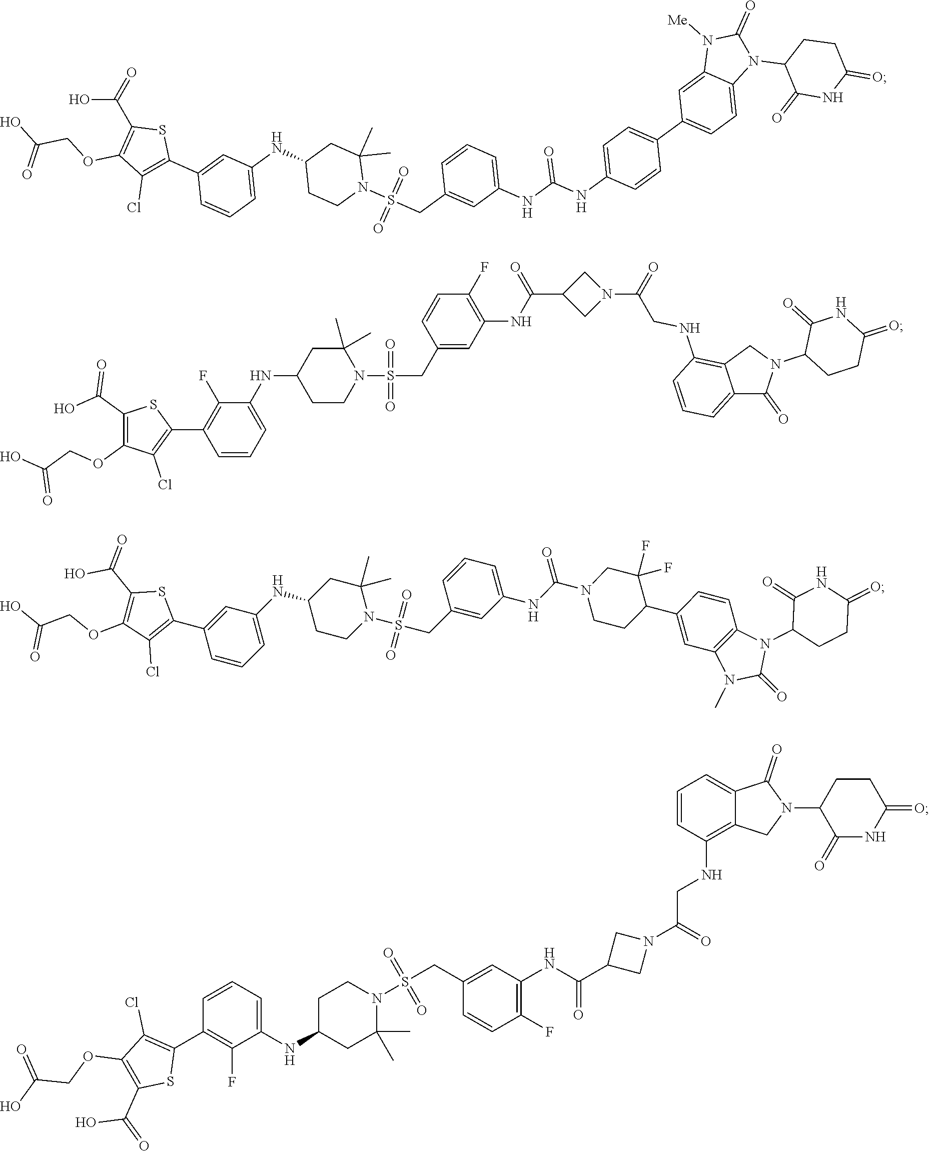 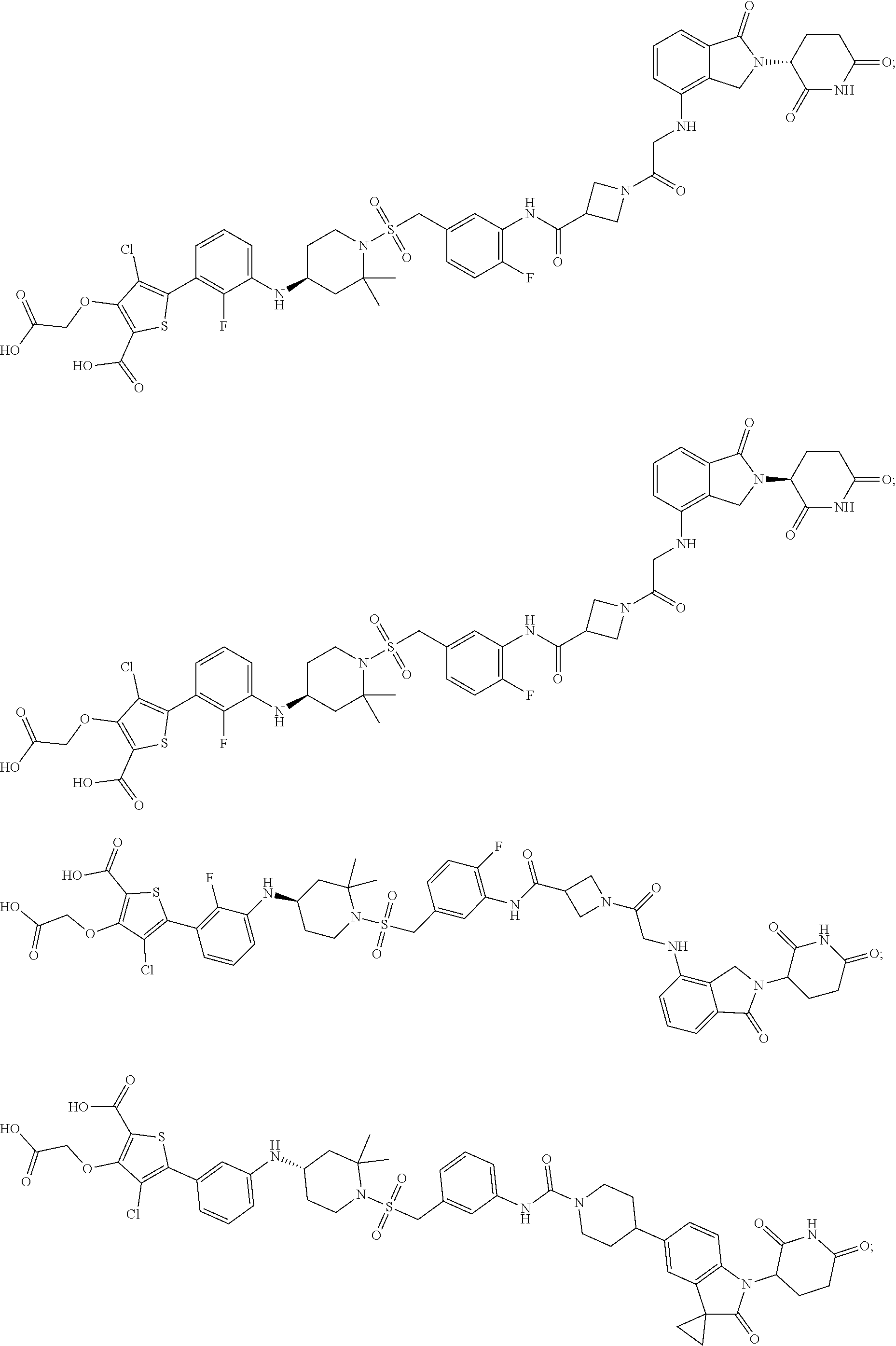 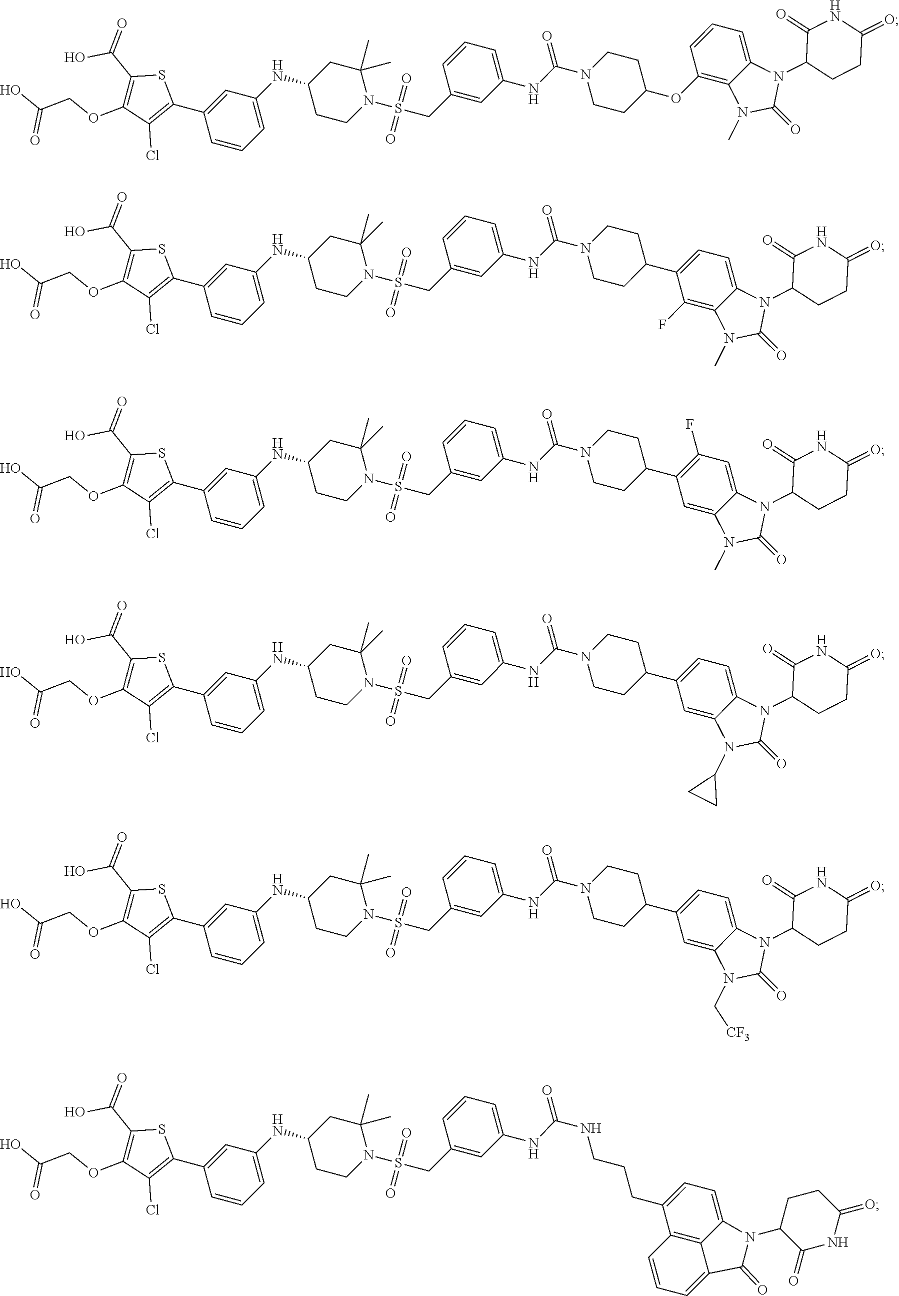 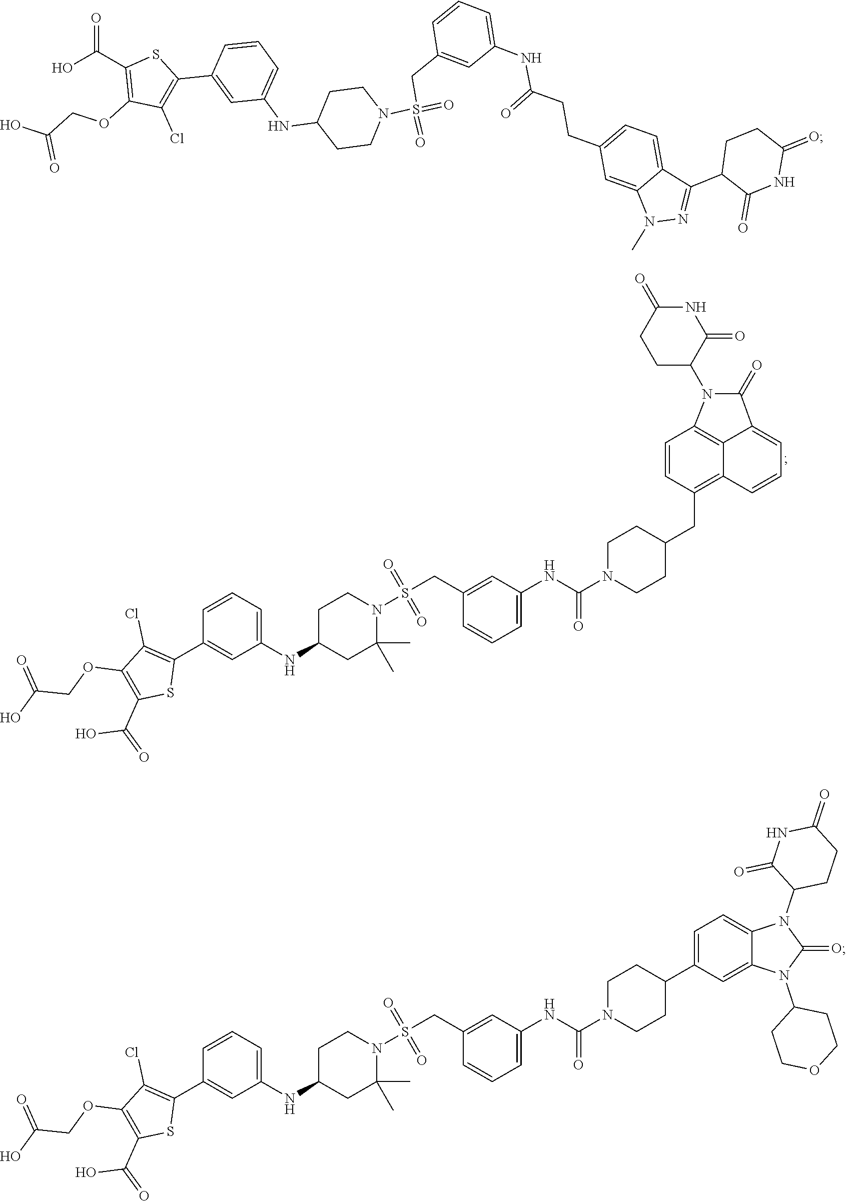 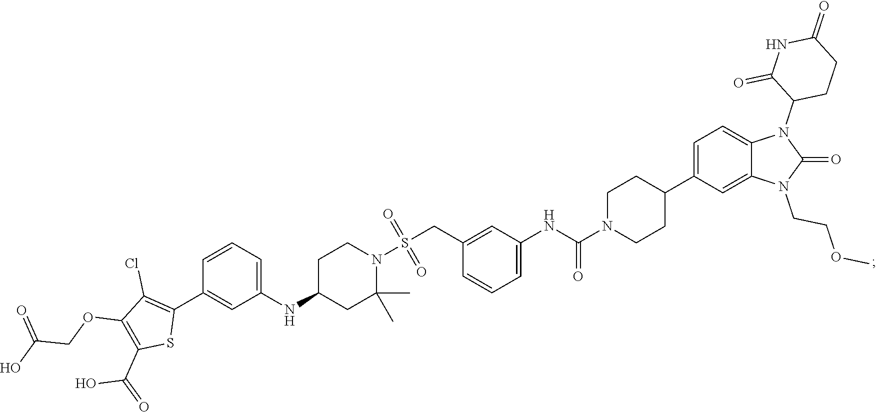 [SMILES: COCCn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] 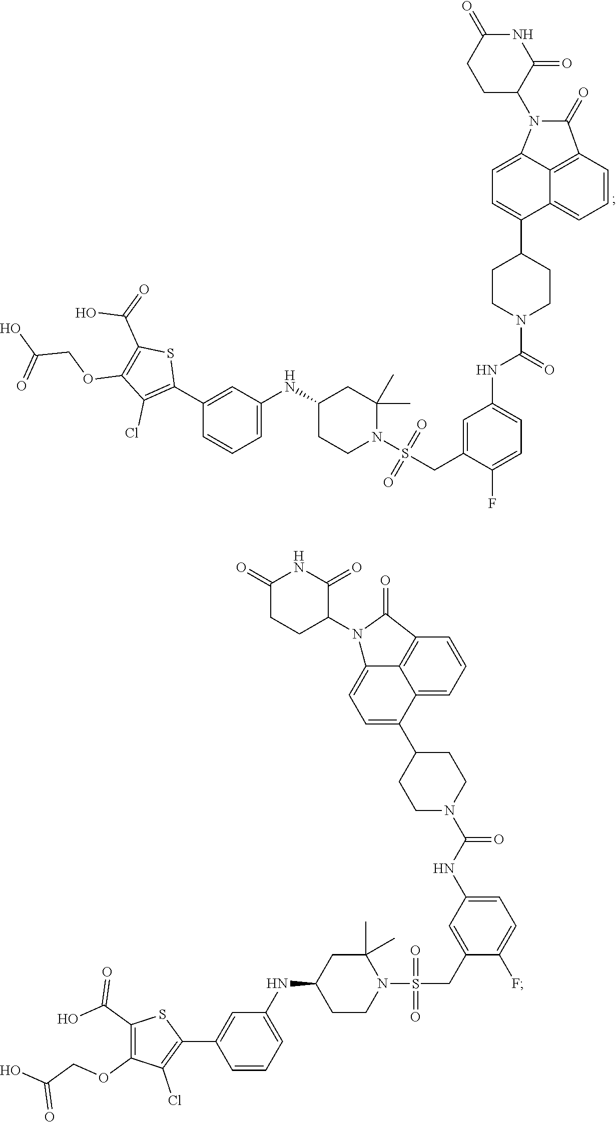 [SMILES: CC1(C)C[C@@H](Nc2cccc(-c3sc(C(=O)O)c(OCC(=O)O)c3Cl)c2)CCN1S(=O)(=O)Cc1cc(NC(=O)N2CCC(c3ccc4c5c(cccc35)C(=O)N4C3CCC(=O)NC3=O)CC2)ccc1F ; CC1(C)C[C@H](Nc2cccc(-c3sc(C(=O)O)c(OCC(=O)O)c3Cl)c2)CCN1S(=O)(=O)Cc1cc(NC(=O)N2CCC(c3ccc4c5c(cccc35)C(=O)N4C3CCC(=O)NC3=O)CC2)ccc1F] 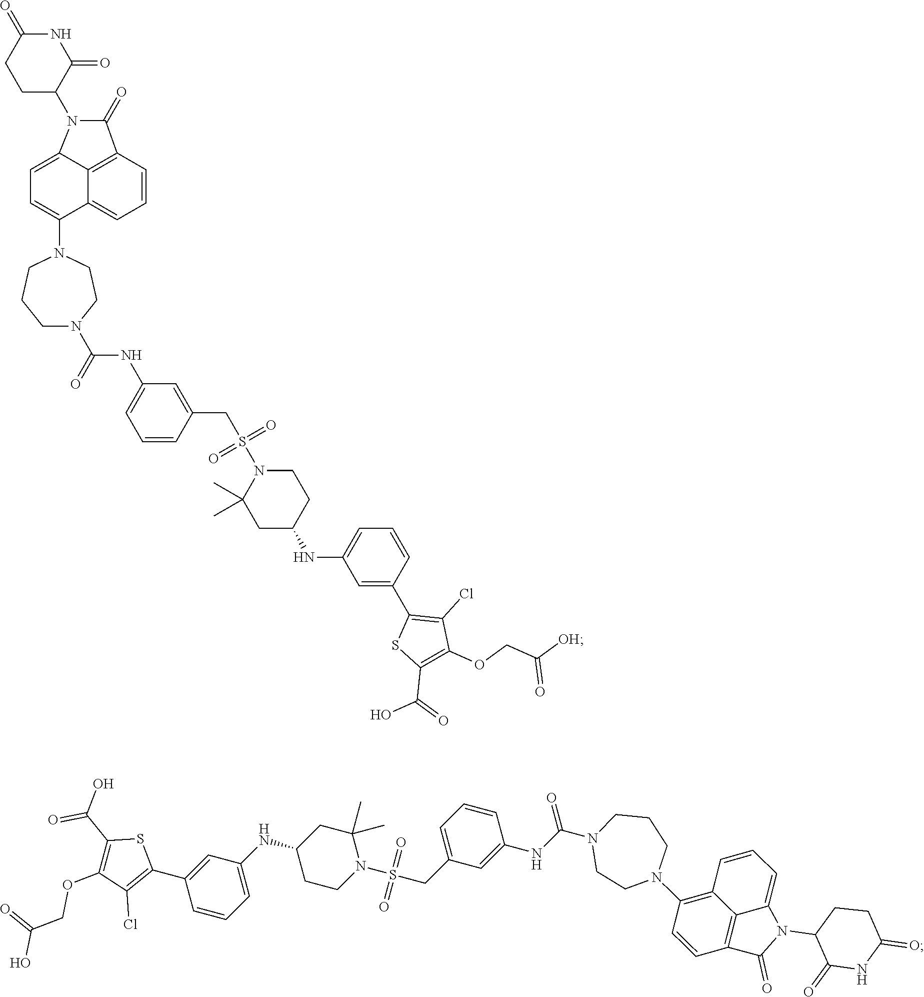 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCCN(c3ccc4c5c(cccc35)C(=O)N4C3CCC(=O)NC3=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCCN(c3ccc4c5c(cccc35)N(C3CCC(=O)NC3=O)C4=O)CC2)c1] 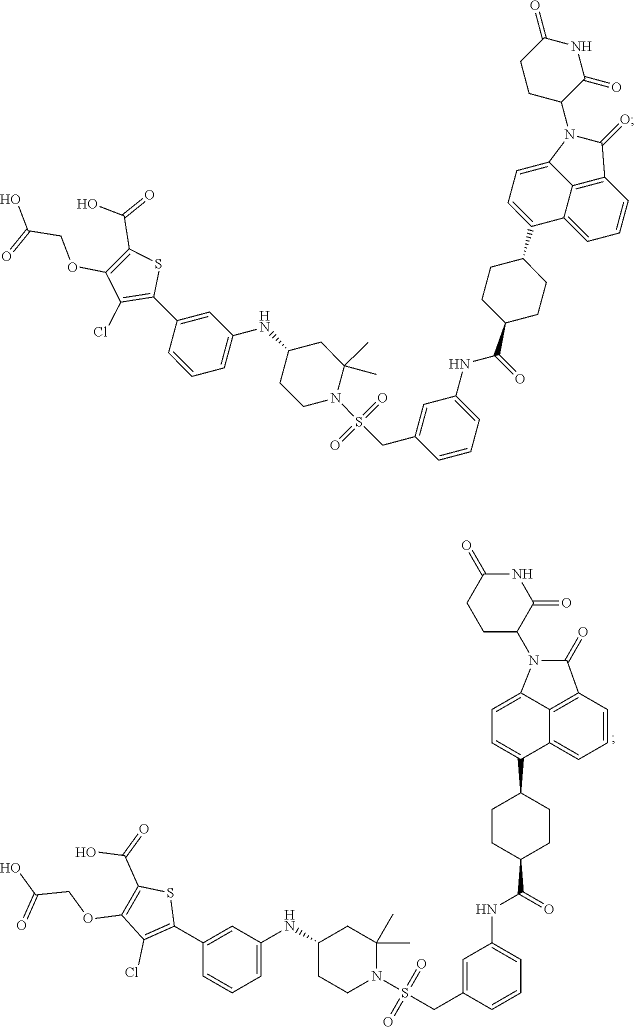 [SMILES: CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@H](c3ccc4c5c(cccc53)C(=O)N4C3CCC(=O)NC3=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)[C@H]2CC[C@@H](c3ccc4c5c(cccc53)C(=O)N4C3CCC(=O)NC3=O)CC2)c1] 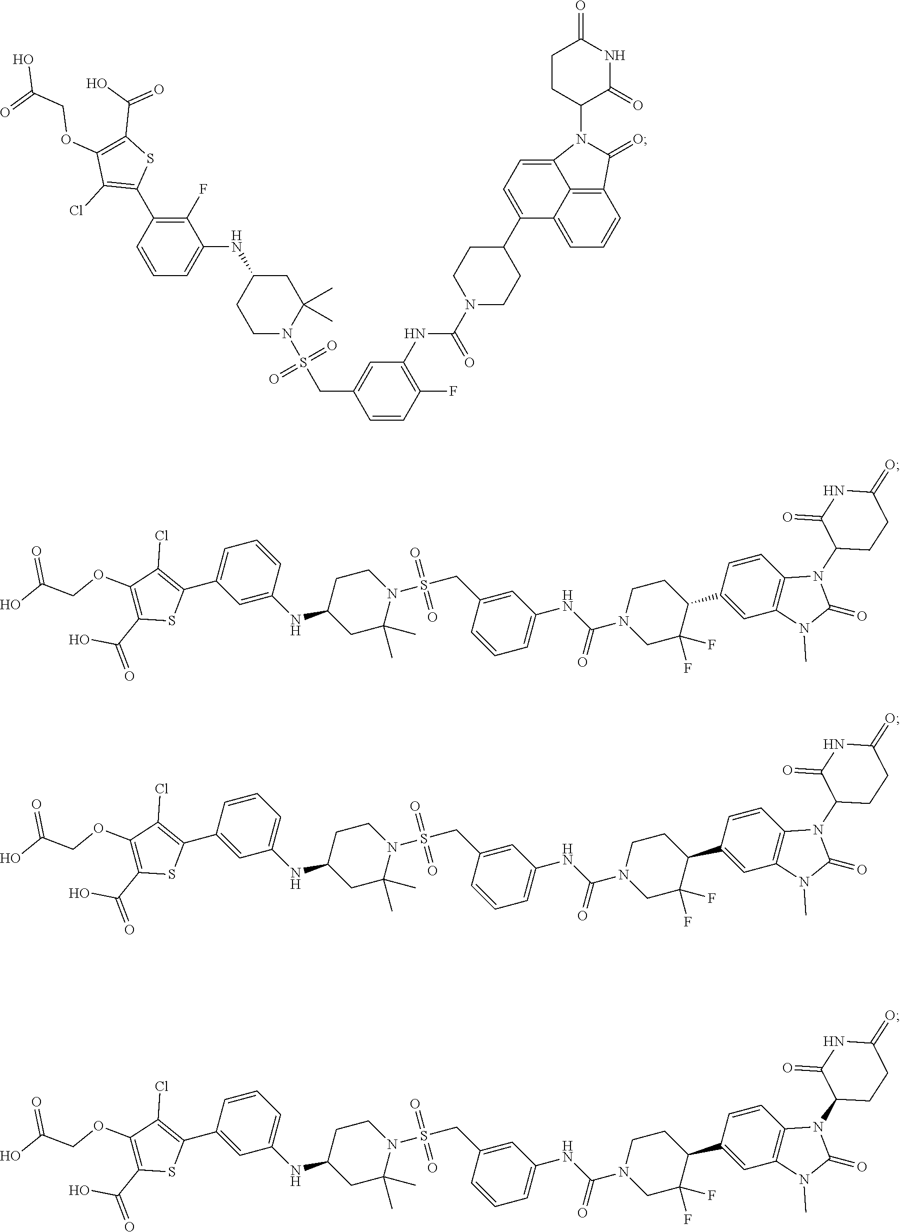 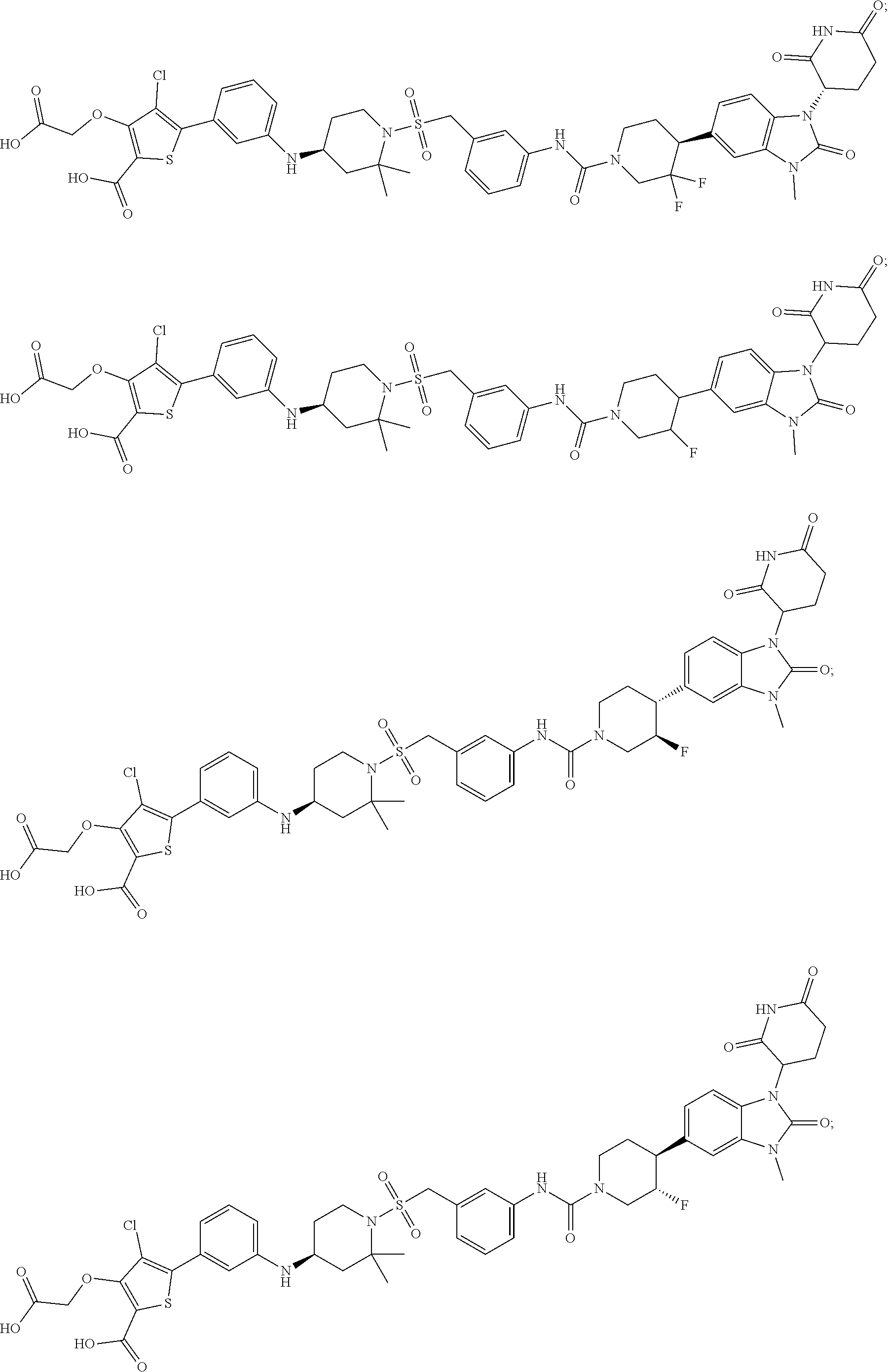 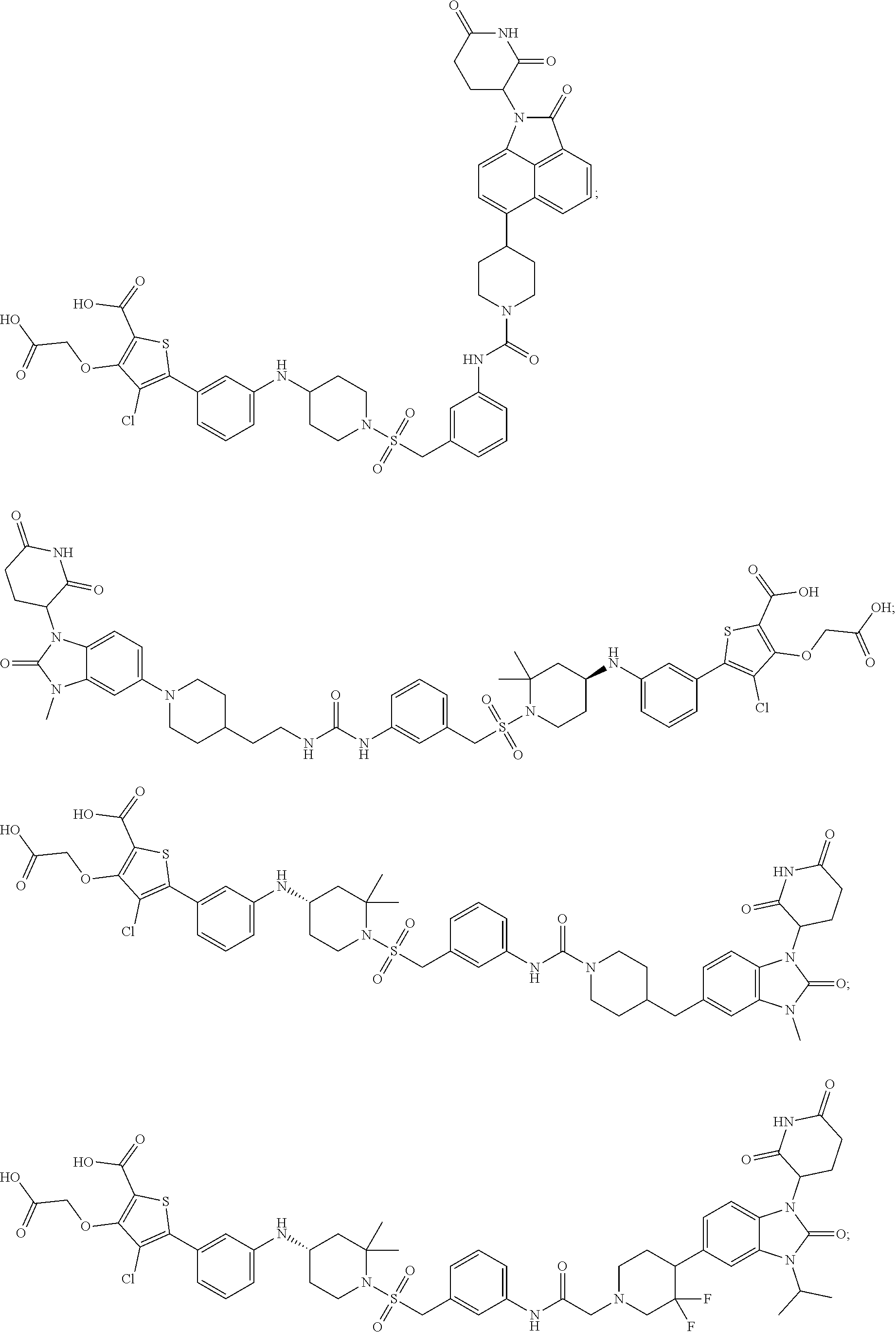 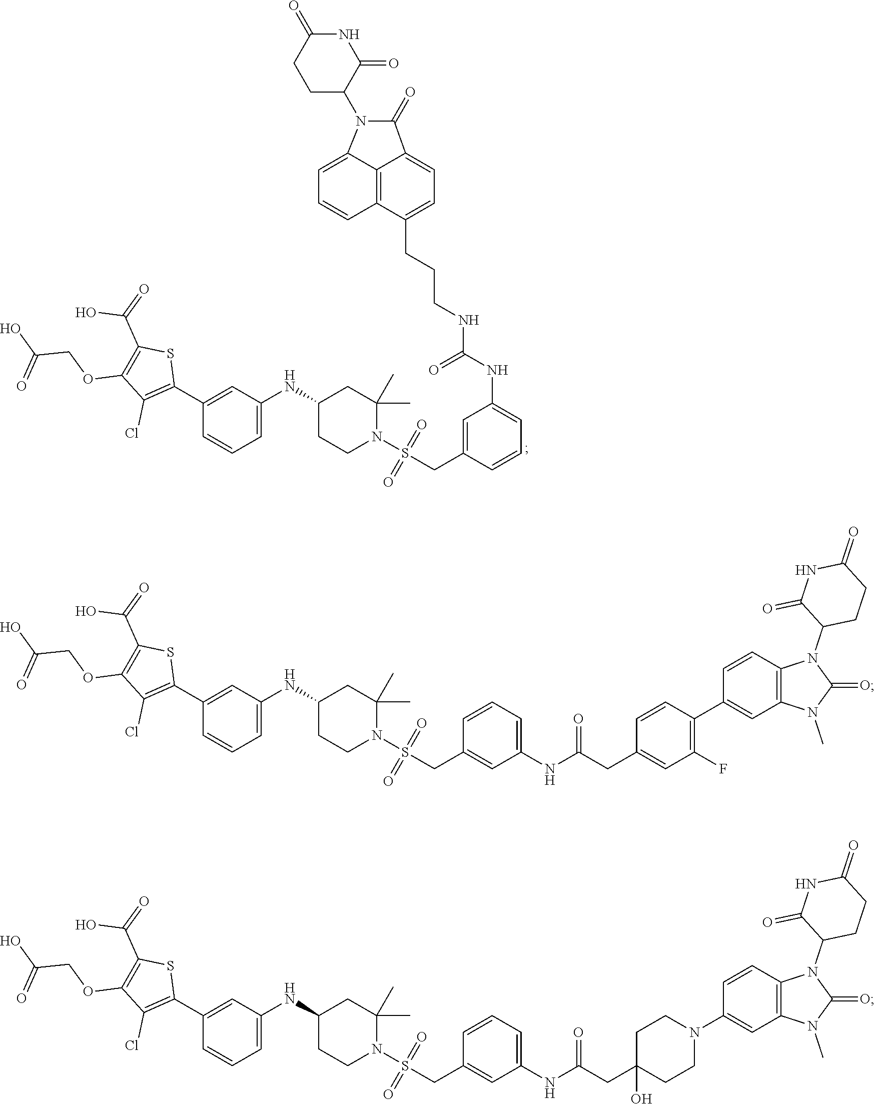 [SMILES: CC1(C)C[C@@H](Nc2cccc(-c3sc(C(=O)O)c(OCC(=O)O)c3Cl)c2)CCN1S(=O)(=O)Cc1cccc(NC(=O)NCCCc2ccc3c4c(cccc24)N(C2CCC(=O)NC2=O)C3=O)c1 ; Cn1c(=O)n(C2CCC(=O)NC2=O)c2ccc(-c3ccc(CC(=O)Nc4cccc(CS(=O)(=O)N5CC[C@H](Nc6cccc(-c7sc(C(=O)O)c(OCC(=O)O)c7Cl)c6)CC5(C)C)c4)cc3F)cc21 ; Cn1c(=O)n(C2CCC(=O)NC2=O)c2ccc(N3CCC(O)(CC(=O)Nc4cccc(CS(=O)(=O)N5CC[C@@H](Nc6cccc(-c7sc(C(=O)O)c(OCC(=O)O)c7Cl)c6)CC5(C)C)c4)CC3)cc21] 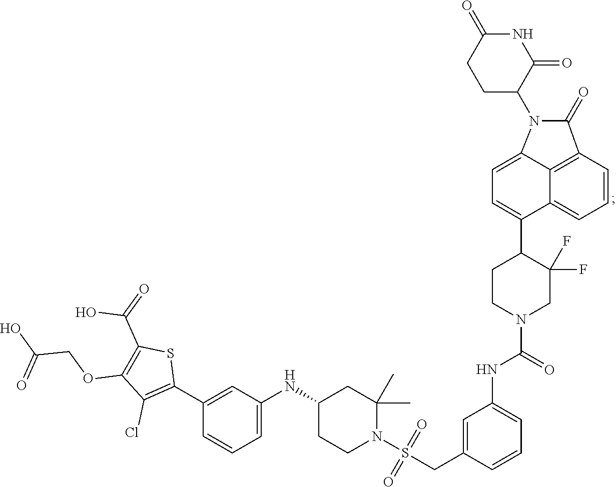 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)C(F)(F)C2)c1] 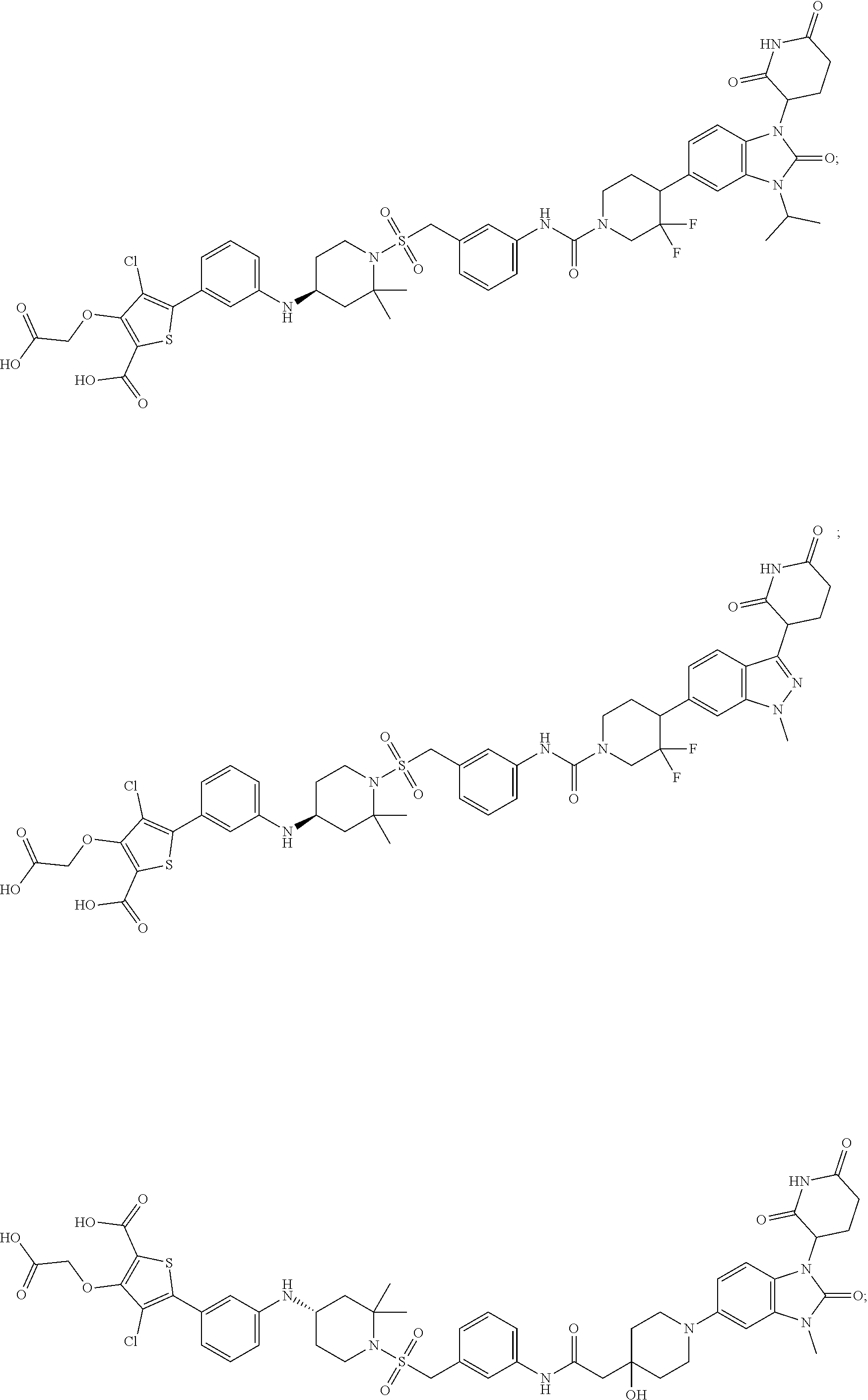 [SMILES: CC(C)n1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21 ; Cn1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21 ; Cn1c(=O)n(C2CCC(=O)NC2=O)c2ccc(N3CCC(O)(CC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] 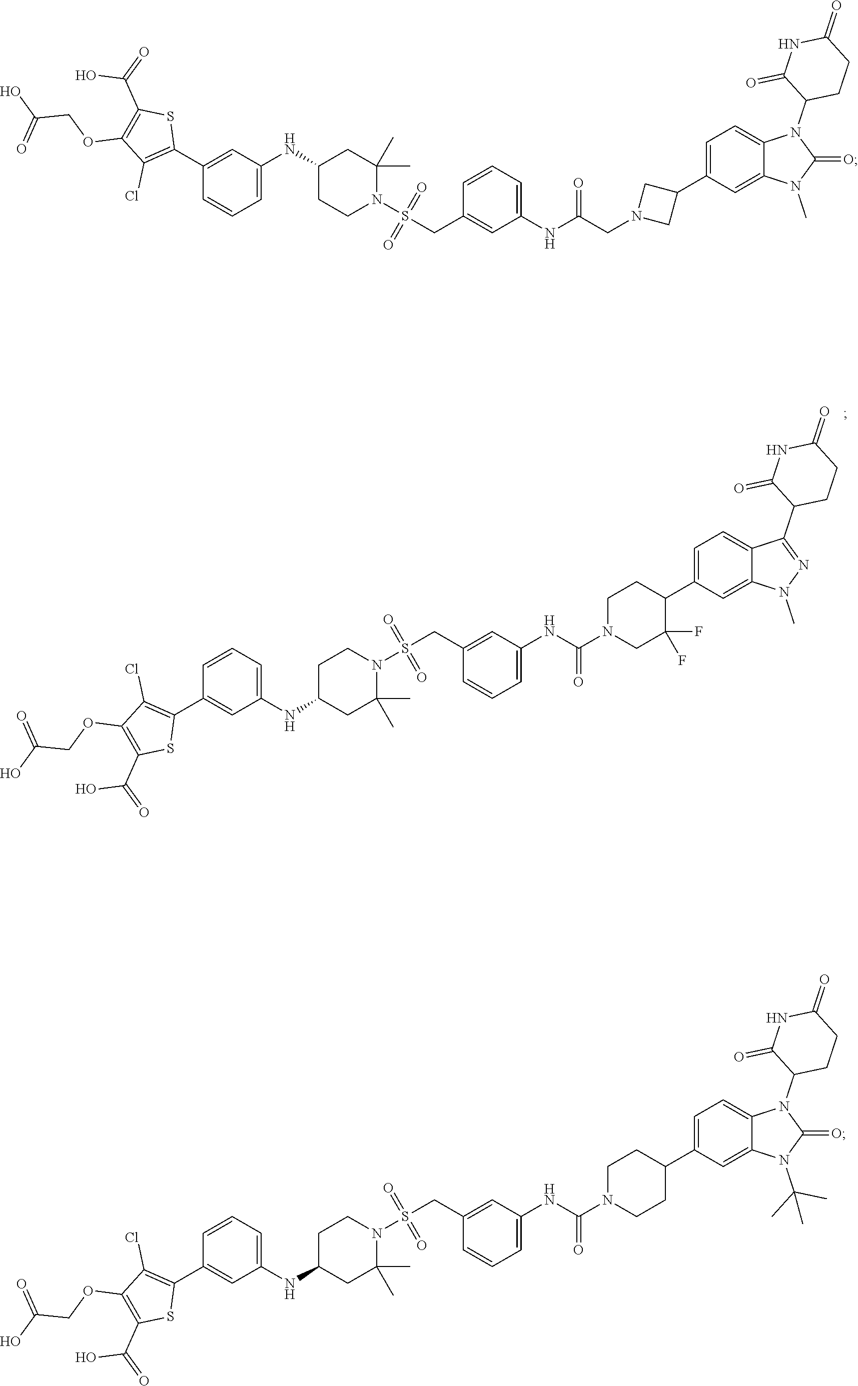 [SMILES: Cn1c(=O)n(C2CCC(=O)NC2=O)c2ccc(C3CN(CC(=O)Nc4cccc(CS(=O)(=O)N5CC[C@H](Nc6cccc(-c7sc(C(=O)O)c(OCC(=O)O)c7Cl)c6)CC5(C)C)c4)C3)cc21 ; Cn1nc(C2CCC(=O)NC2=O)c2ccc(C3CCN(C(=O)Nc4cccc(CS(=O)(=O)N5CC[C@@H](Nc6cccc(-c7sc(C(=O)O)c(OCC(=O)O)c7Cl)c6)CC5(C)C)c4)CC3(F)F)cc21 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C(C)(C)C)c(=O)n4C3CCC(=O)NC3=O)CC2)c1] 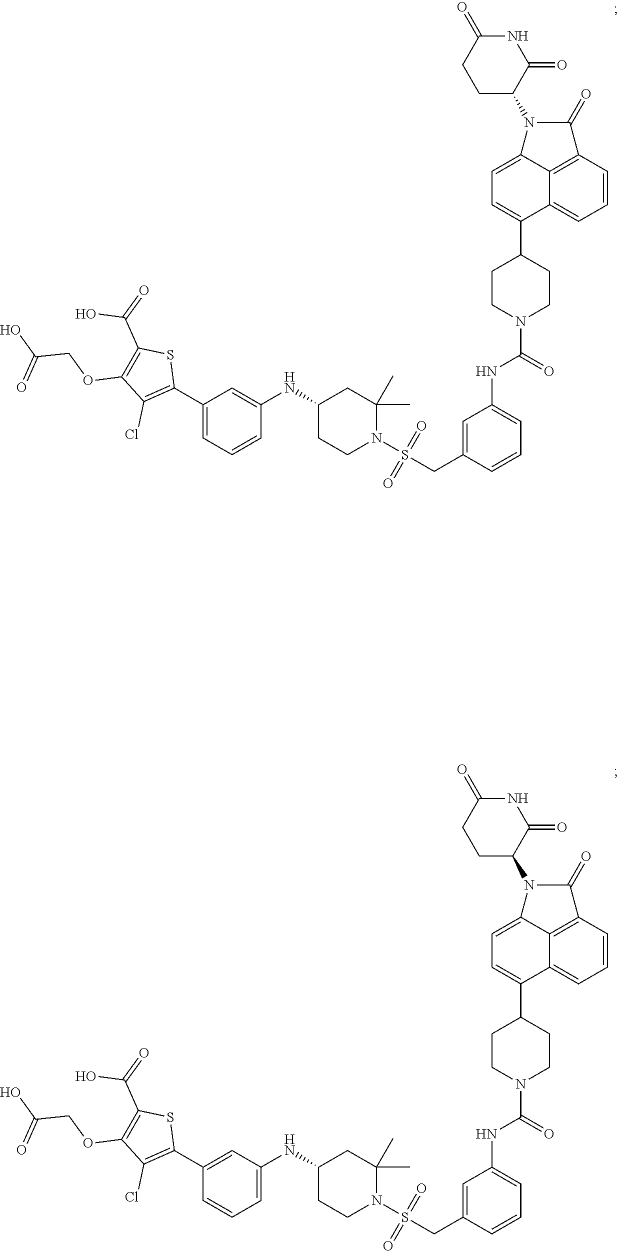 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4[C@@H]3CCC(=O)NC3=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4[C@H]3CCC(=O)NC3=O)CC2)c1] 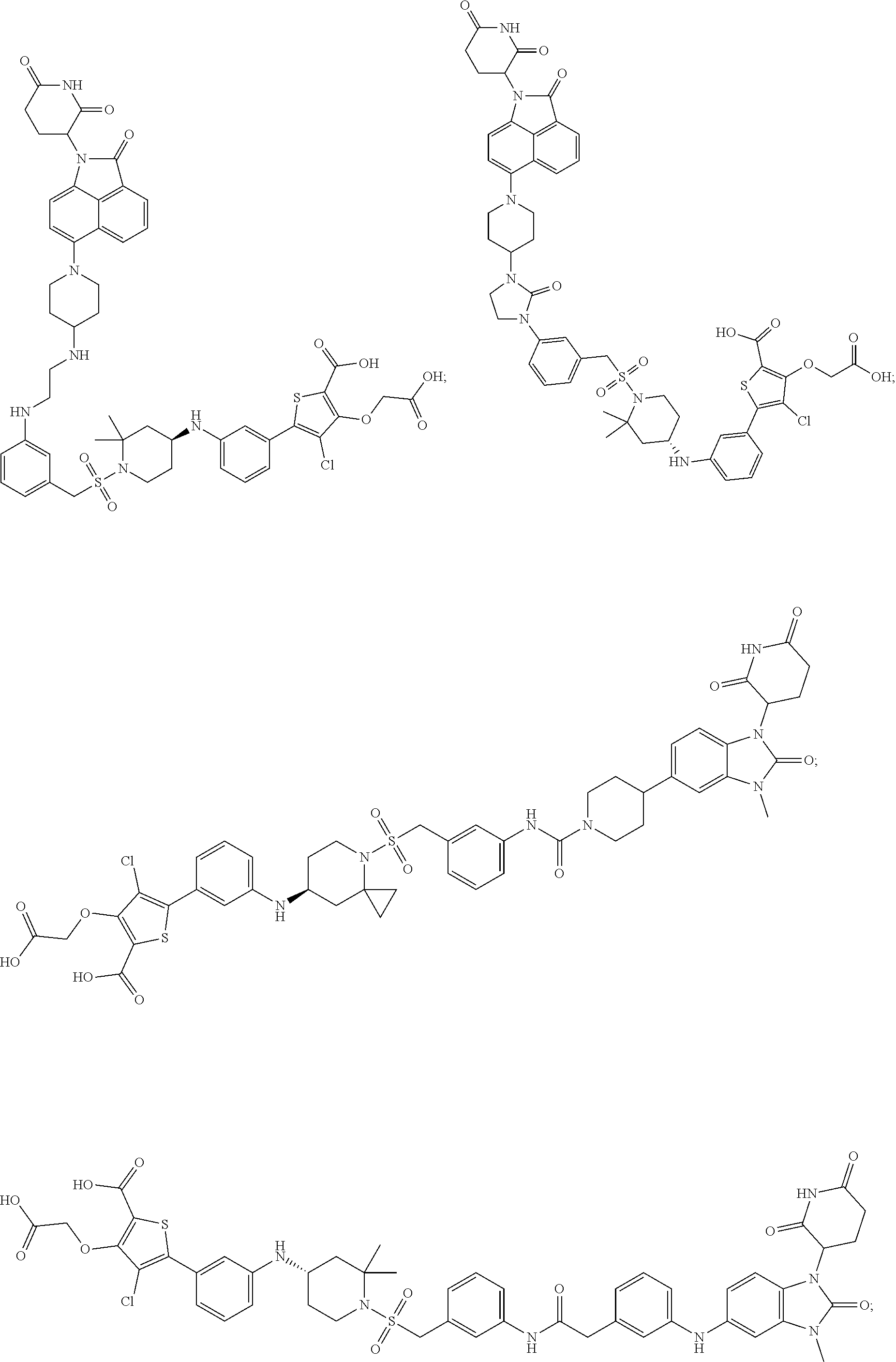 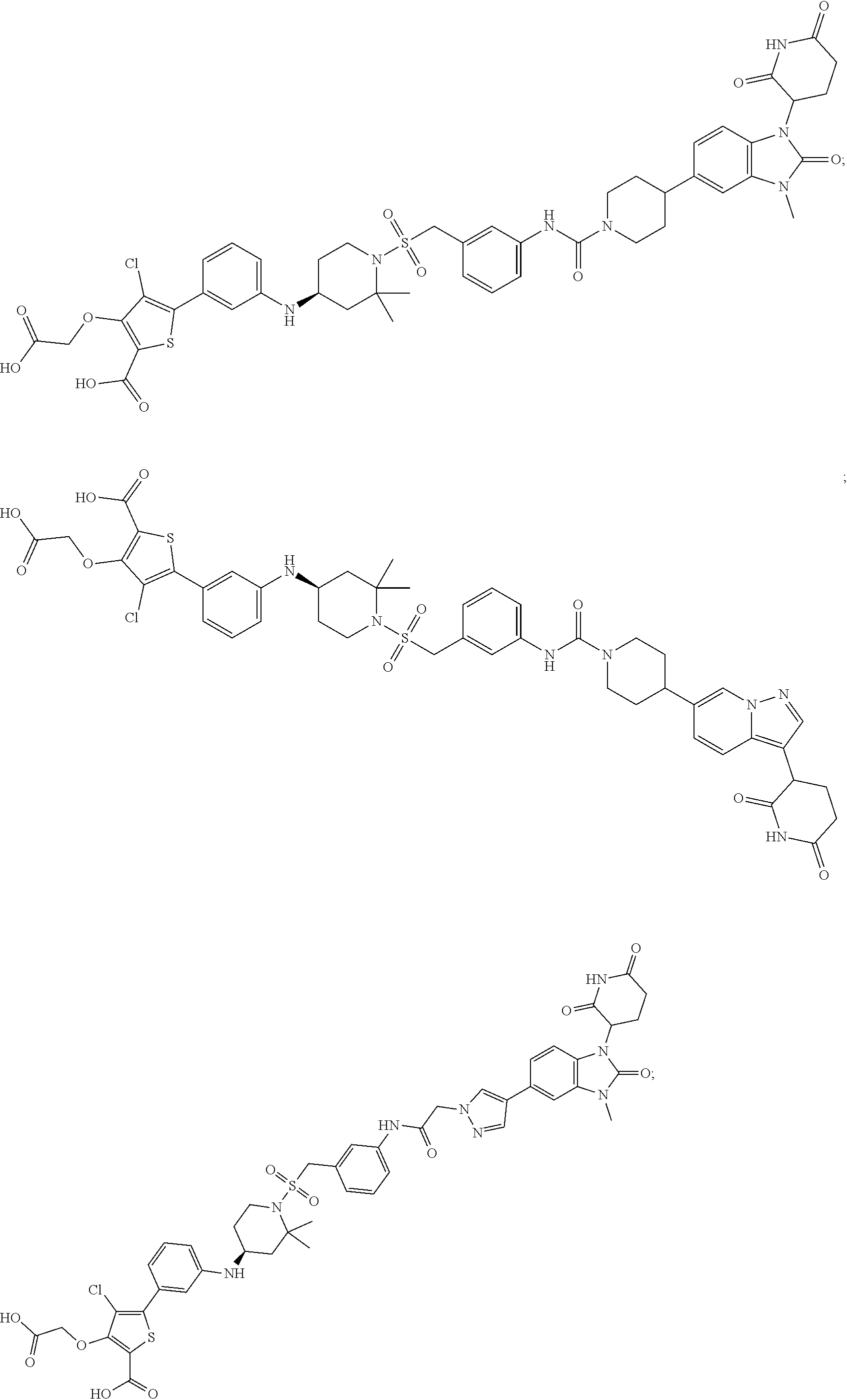 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c(C5CCC(=O)NC5=O)cnn4c3)CC2)c1 ; Cn1c(=O)n(C2CCC(=O)NC2=O)c2ccc(-c3cnn(CC(=O)Nc4cccc(CS(=O)(=O)N5CC[C@H](Nc6cccc(-c7sc(C(=O)O)c(OCC(=O)O)c7Cl)c6)CC5(C)C)c4)c3)cc21] 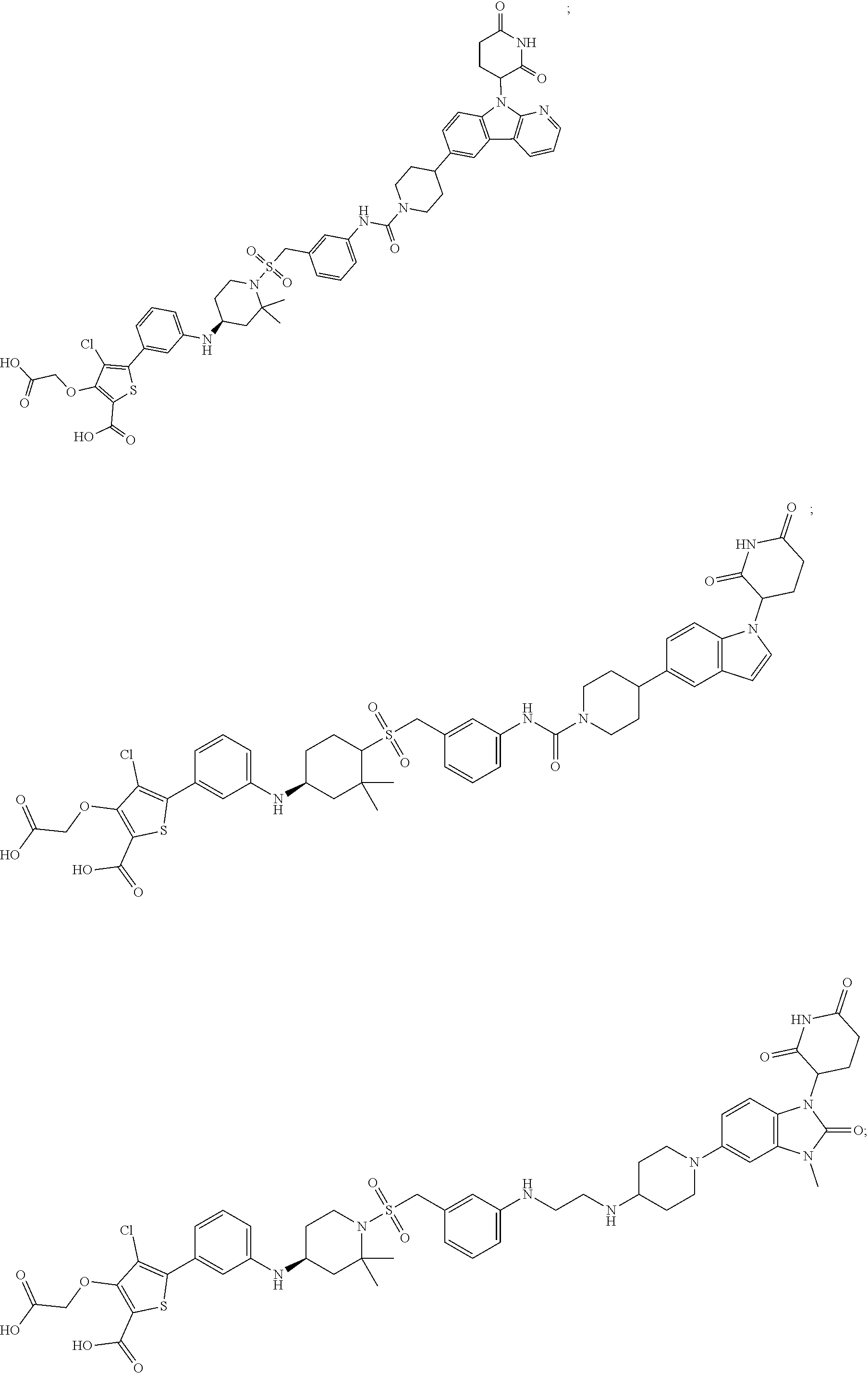 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c(c3)c3cccnc3n4C3CCC(=O)NC3=O)CC2)c1 ; CC1(C)C[C@@H](Nc2cccc(-c3sc(C(=O)O)c(OCC(=O)O)c3Cl)c2)CCC1S(=O)(=O)Cc1cccc(NC(=O)N2CCC(c3ccc4c(ccn4C4CCC(=O)NC4=O)c3)CC2)c1 ; Cn1c(=O)n(C2CCC(=O)NC2=O)c2ccc(N3CCC(NCCNc4cccc(CS(=O)(=O)N5CC[C@H](Nc6cccc(-c7sc(C(=O)O)c(OCC(=O)O)c7Cl)c6)CC5(C)C)c4)CC3)cc21] 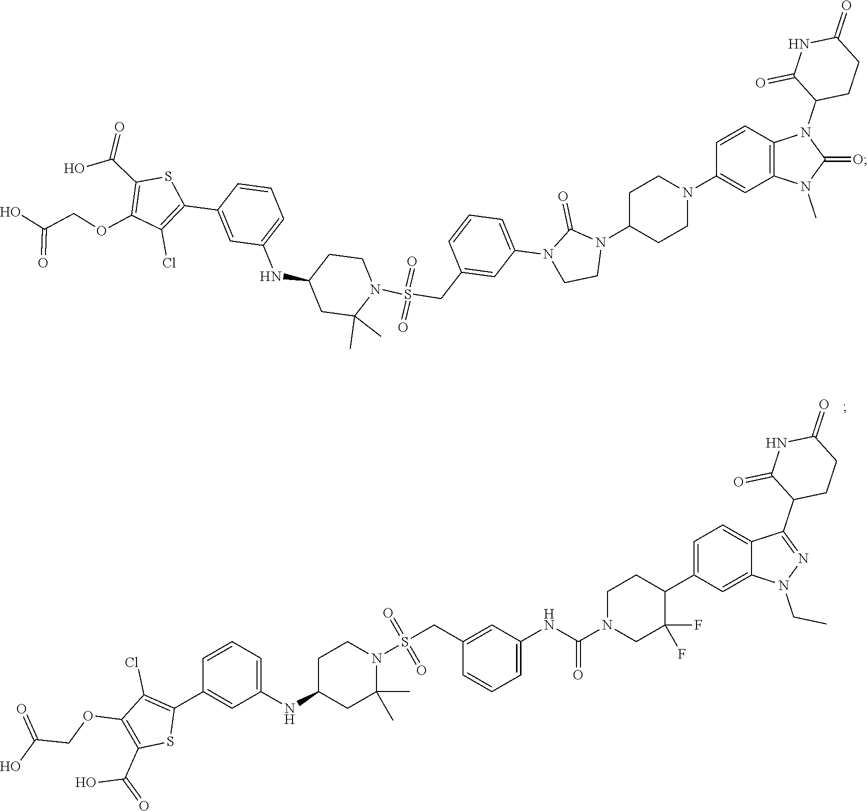 [SMILES: Cn1c(=O)n(C2CCC(=O)NC2=O)c2ccc(N3CCC(N4CCN(c5cccc(CS(=O)(=O)N6CC[C@H](Nc7cccc(-c8sc(C(=O)O)c(OCC(=O)O)c8Cl)c7)CC6(C)C)c5)C4=O)CC3)cc21 ; CCn1nc(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3(F)F)cc21] 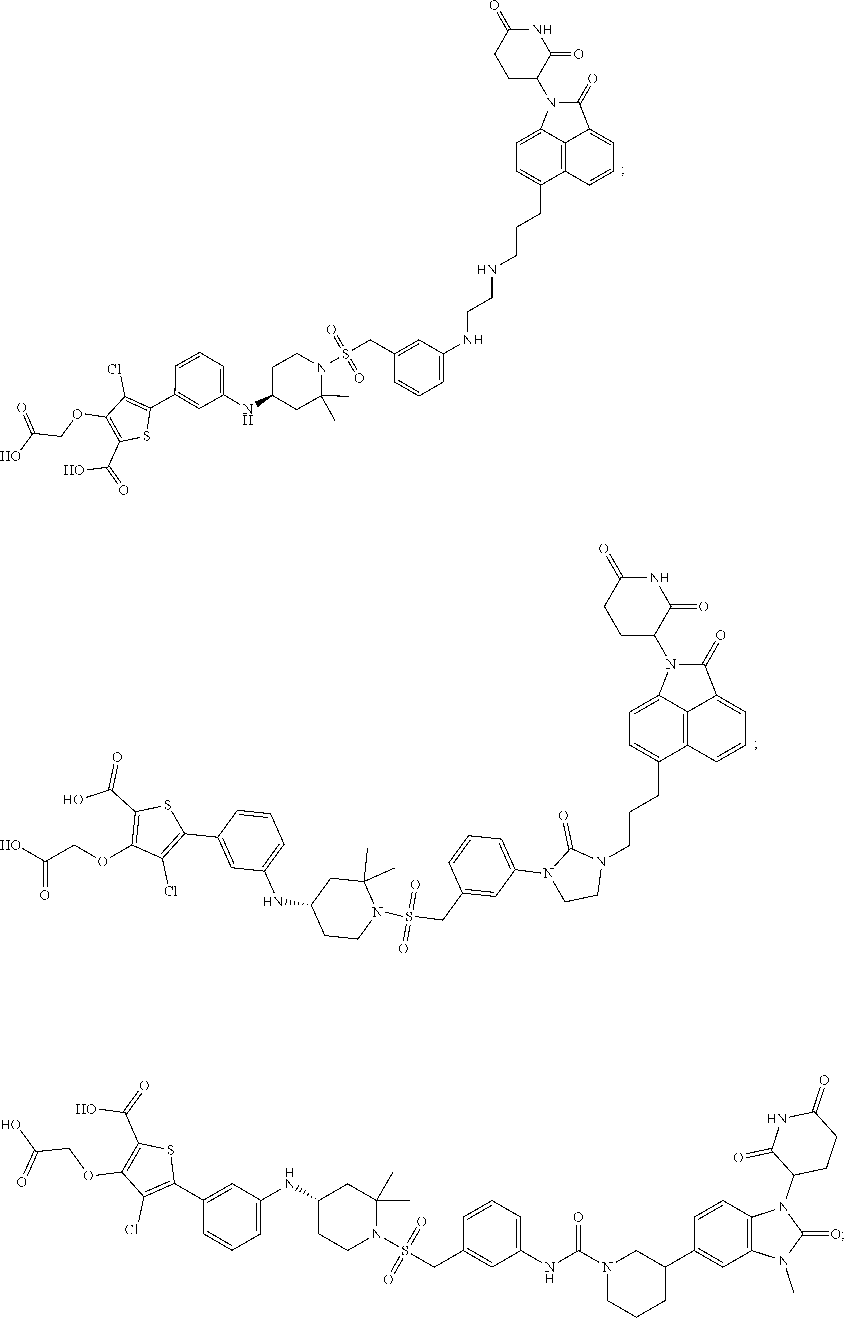 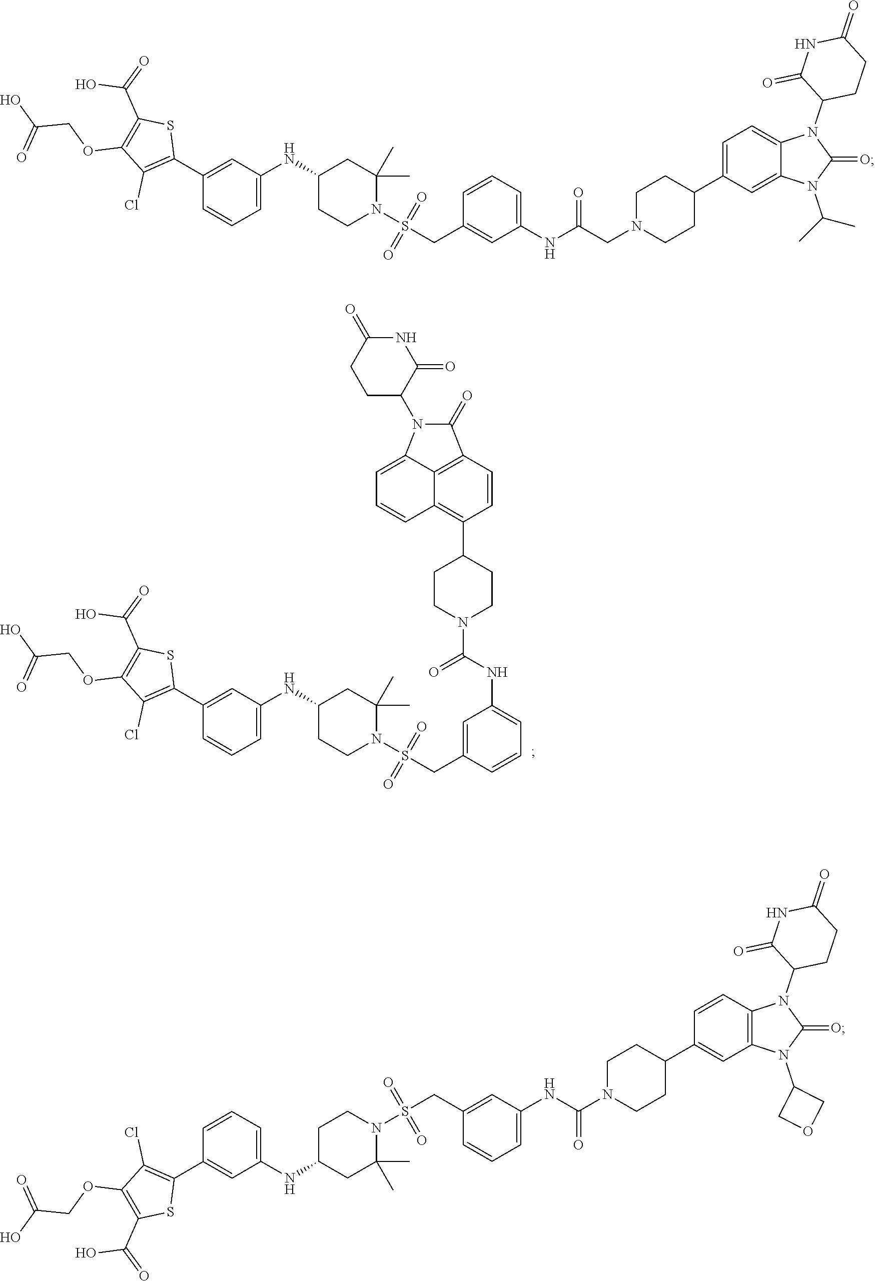 [SMILES: CC(C)n1c(=O)n(C2CCC(=O)NC2=O)c2ccc(C3CCN(CC(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21 ; CC1(C)C[C@@H](Nc2cccc(-c3sc(C(=O)O)c(OCC(=O)O)c3Cl)c2)CCN1S(=O)(=O)Cc1cccc(NC(=O)N2CCC(c3ccc4c5c(cccc35)N(C3CCC(=O)NC3=O)C4=O)CC2)c1 ; CC1(C)C[C@H](Nc2cccc(-c3sc(C(=O)O)c(OCC(=O)O)c3Cl)c2)CCN1S(=O)(=O)Cc1cccc(NC(=O)N2CCC(c3ccc4c(c3)n(C3COC3)c(=O)n4C3CCC(=O)NC3=O)CC2)c1] 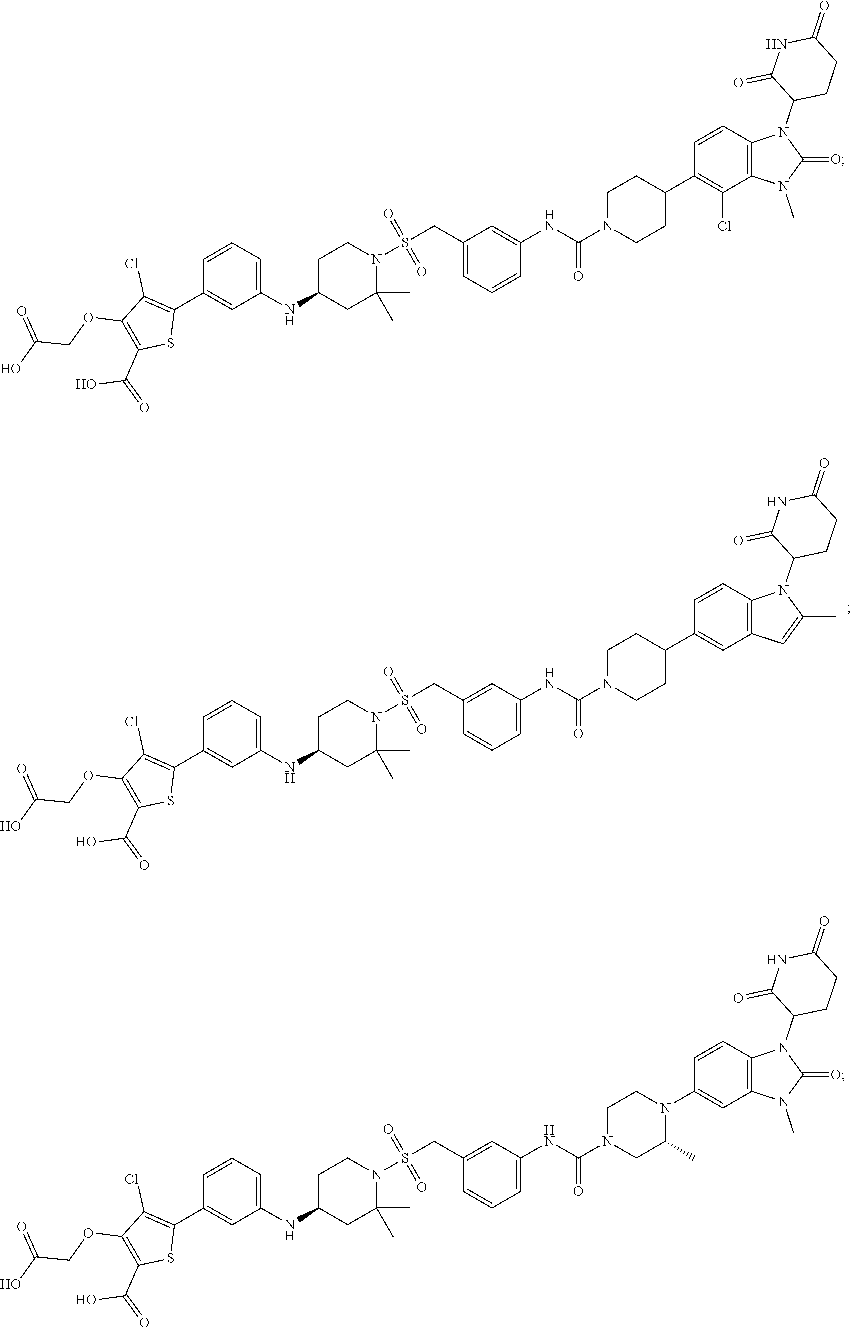 [SMILES: Cn1c(=O)n(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)c(Cl)c21 ; Cc1cc2cc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)ccc2n1C1CCC(=O)NC1=O ; C[C@@H]1CN(C(=O)Nc2cccc(CS(=O)(=O)N3CC[C@H](Nc4cccc(-c5sc(C(=O)O)c(OCC(=O)O)c5Cl)c4)CC3(C)C)c2)CCN1c1ccc2c(c1)n(C)c(=O)n2C1CCC(=O)NC1=O] 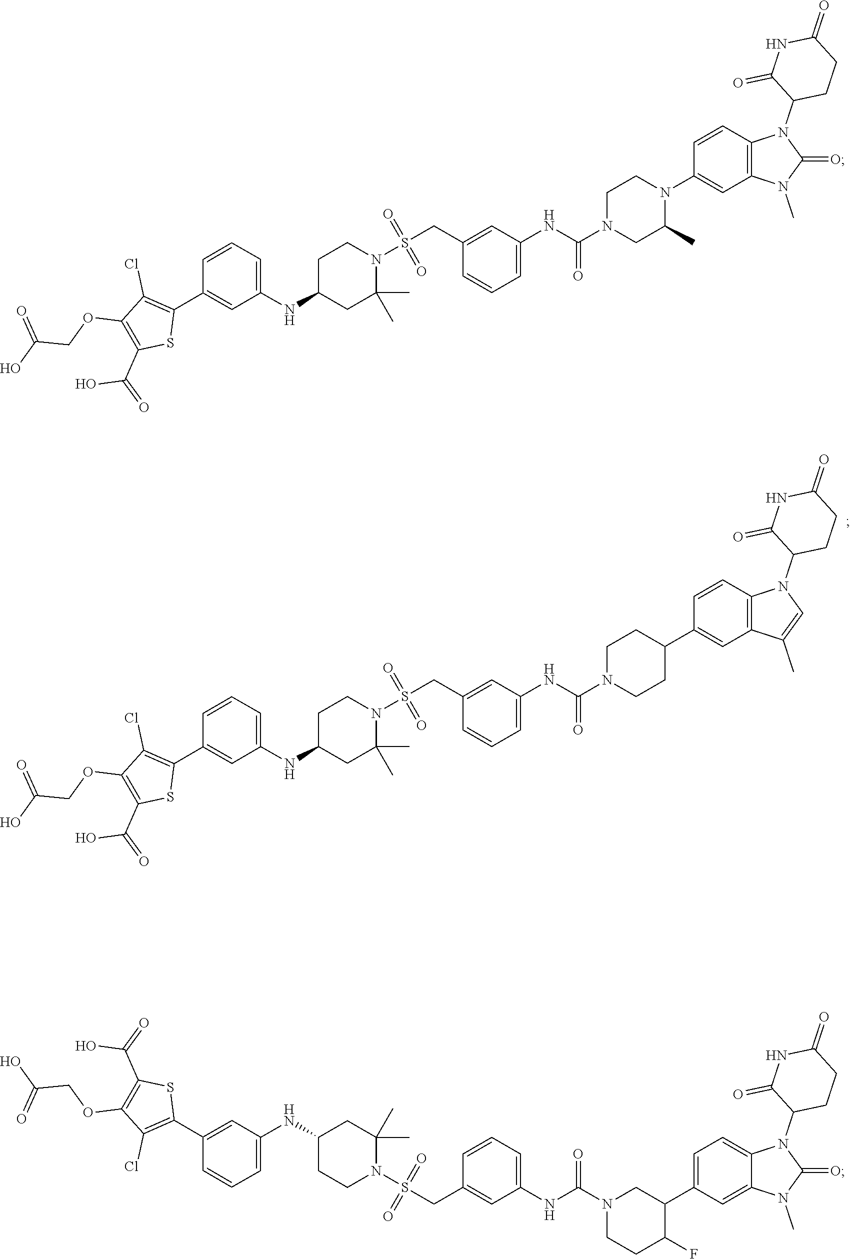 [SMILES: C[C@H]1CN(C(=O)Nc2cccc(CS(=O)(=O)N3CC[C@H](Nc4cccc(-c5sc(C(=O)O)c(OCC(=O)O)c5Cl)c4)CC3(C)C)c2)CCN1c1ccc2c(c1)n(C)c(=O)n2C1CCC(=O)NC1=O ; Cc1cn(C2CCC(=O)NC2=O)c2ccc(C3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc12 ; Cn1c(=O)n(C2CCC(=O)NC2=O)c2ccc(C3CN(C(=O)Nc4cccc(CS(=O)(=O)N5CC[C@H](Nc6cccc(-c7sc(C(=O)O)c(OCC(=O)O)c7Cl)c6)CC5(C)C)c4)CCC3F)cc21] 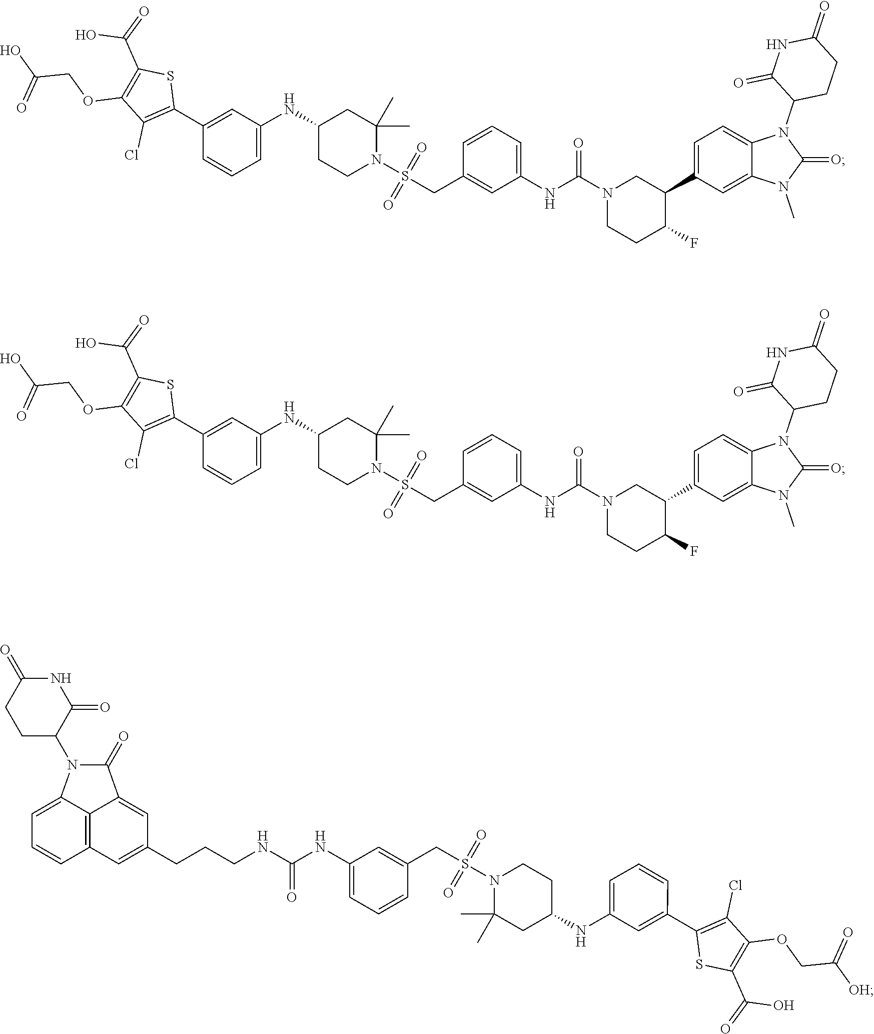 [SMILES: Cn1c(=O)n(C2CCC(=O)NC2=O)c2ccc([C@@H]3CN(C(=O)Nc4cccc(CS(=O)(=O)N5CC[C@H](Nc6cccc(-c7sc(C(=O)O)c(OCC(=O)O)c7Cl)c6)CC5(C)C)c4)CC[C@H]3F)cc21 ; Cn1c(=O)n(C2CCC(=O)NC2=O)c2ccc([C@H]3CN(C(=O)Nc4cccc(CS(=O)(=O)N5CC[C@H](Nc6cccc(-c7sc(C(=O)O)c(OCC(=O)O)c7Cl)c6)CC5(C)C)c4)CC[C@@H]3F)cc21 ; CC1(C)C[C@@H](Nc2cccc(-c3sc(C(=O)O)c(OCC(=O)O)c3Cl)c2)CCN1S(=O)(=O)Cc1cccc(NC(=O)NCCCc2cc3c4c(cccc4c2)N(C2CCC(=O)NC2=O)C3=O)c1] 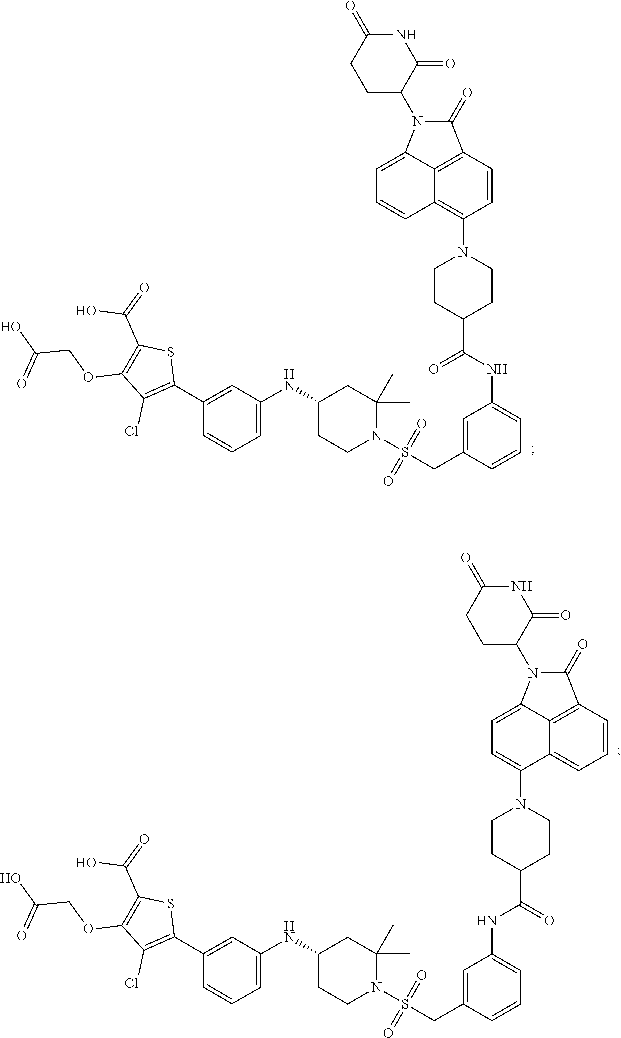 [SMILES: CC1(C)C[C@@H](Nc2cccc(-c3sc(C(=O)O)c(OCC(=O)O)c3Cl)c2)CCN1S(=O)(=O)Cc1cccc(NC(=O)C2CCN(c3ccc4c5c(cccc35)N(C3CCC(=O)NC3=O)C4=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)C2CCN(c3ccc4c5c(cccc35)C(=O)N4C3CCC(=O)NC3=O)CC2)c1] 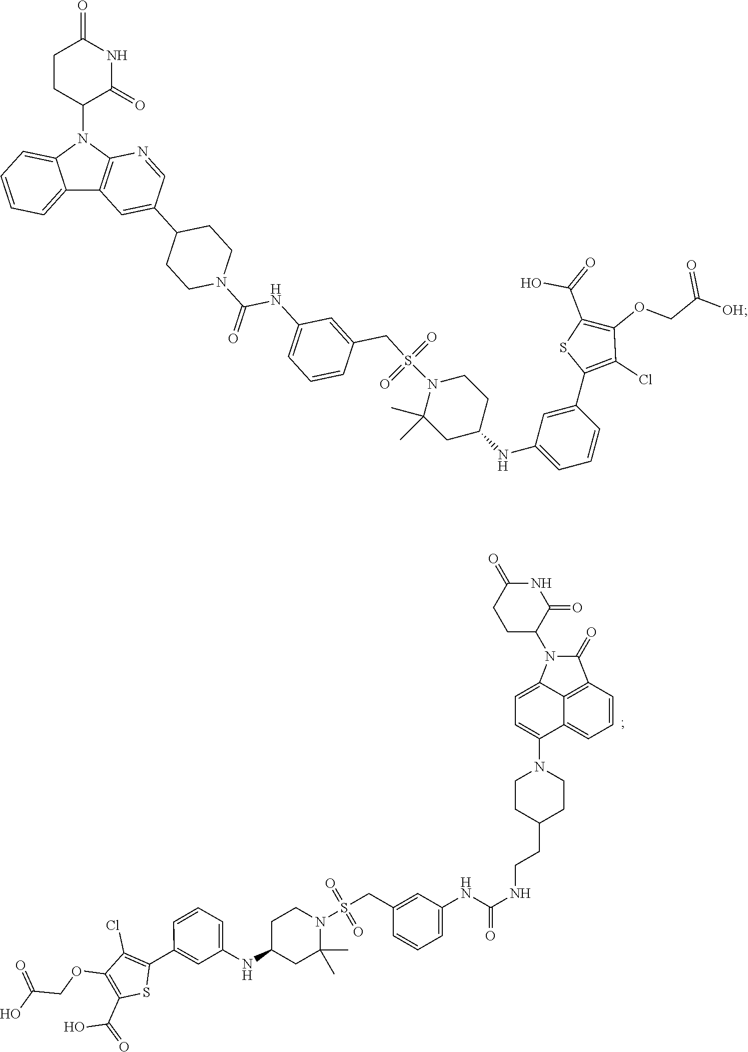 [SMILES: CC1(C)C[C@@H](Nc2cccc(-c3sc(C(=O)O)c(OCC(=O)O)c3Cl)c2)CCN1S(=O)(=O)Cc1cccc(NC(=O)N2CCC(c3cnc4c(c3)c3ccccc3n4C3CCC(=O)NC3=O)CC2)c1 ; CC1(C)C[C@@H](Nc2cccc(-c3sc(C(=O)O)c(OCC(=O)O)c3Cl)c2)CCN1S(=O)(=O)Cc1cccc(NC(=O)NCCC2CCN(c3ccc4c5c(cccc35)C(=O)N4C3CCC(=O)NC3=O)CC2)c1] 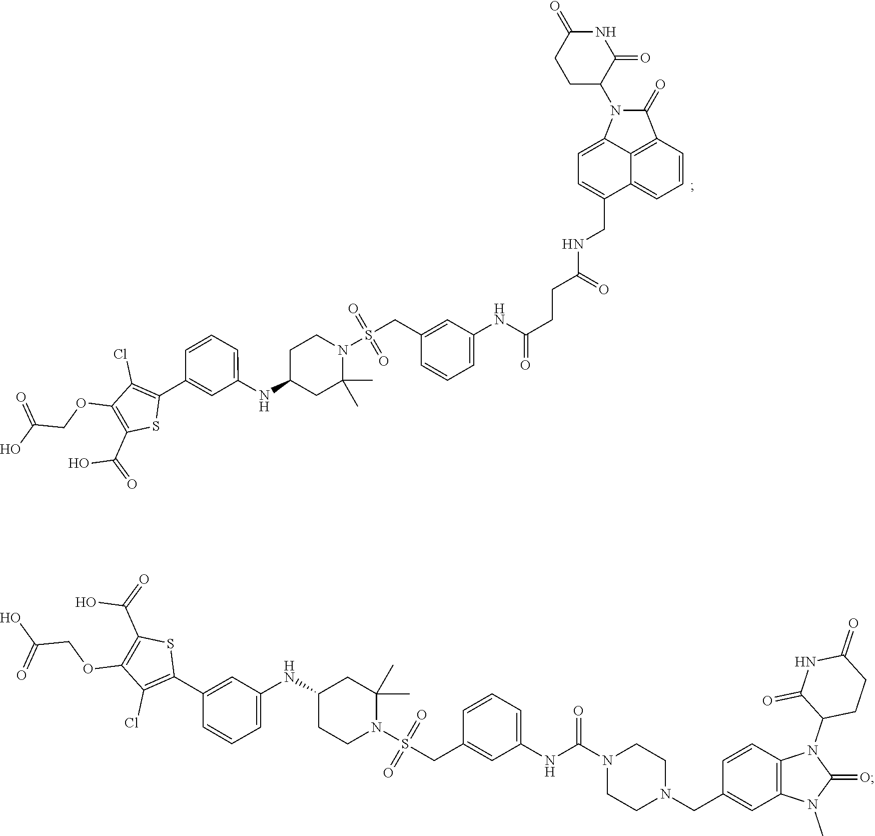 [SMILES: CC1(C)C[C@@H](Nc2cccc(-c3sc(C(=O)O)c(OCC(=O)O)c3Cl)c2)CCN1S(=O)(=O)Cc1cccc(NC(=O)CCC(=O)NCc2ccc3c4c(cccc24)C(=O)N3C2CCC(=O)NC2=O)c1 ; Cn1c(=O)n(C2CCC(=O)NC2=O)c2ccc(CN3CCN(C(=O)Nc4cccc(CS(=O)(=O)N5CC[C@H](Nc6cccc(-c7sc(C(=O)O)c(OCC(=O)O)c7Cl)c6)CC5(C)C)c4)CC3)cc21] 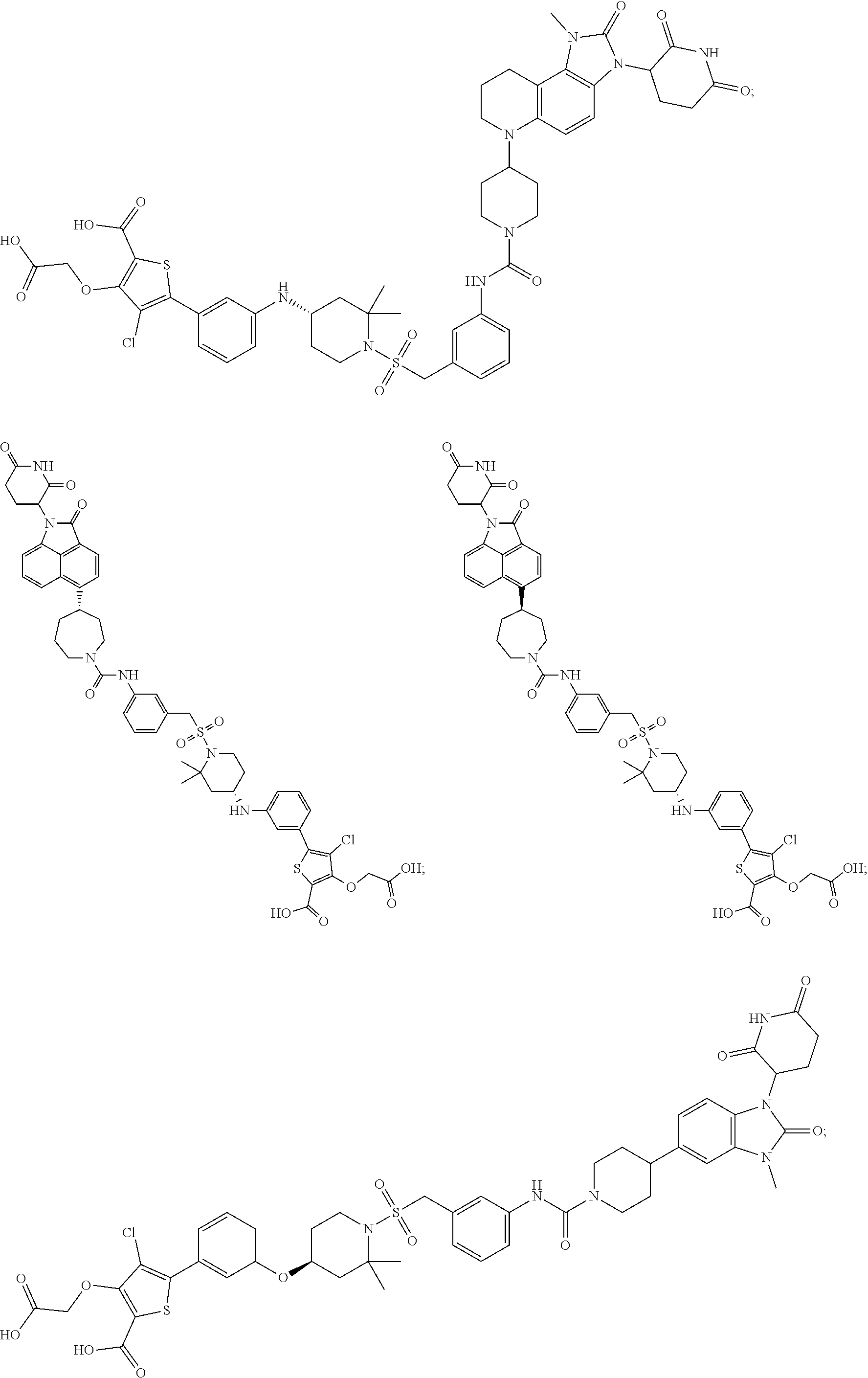 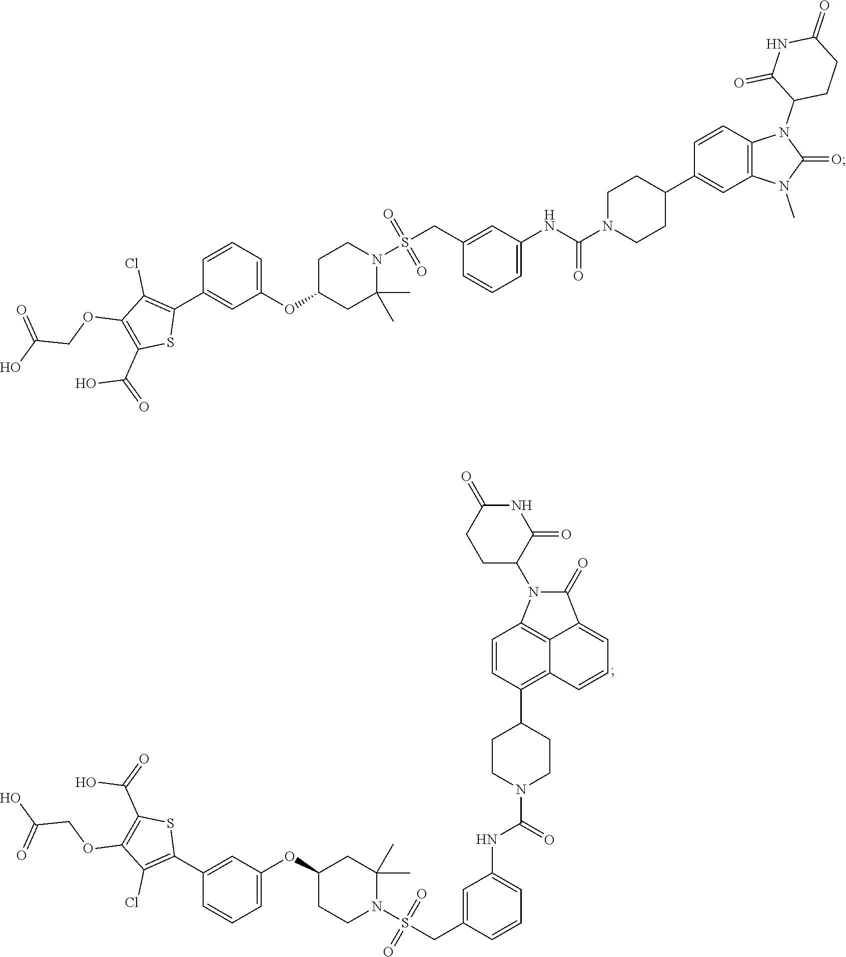 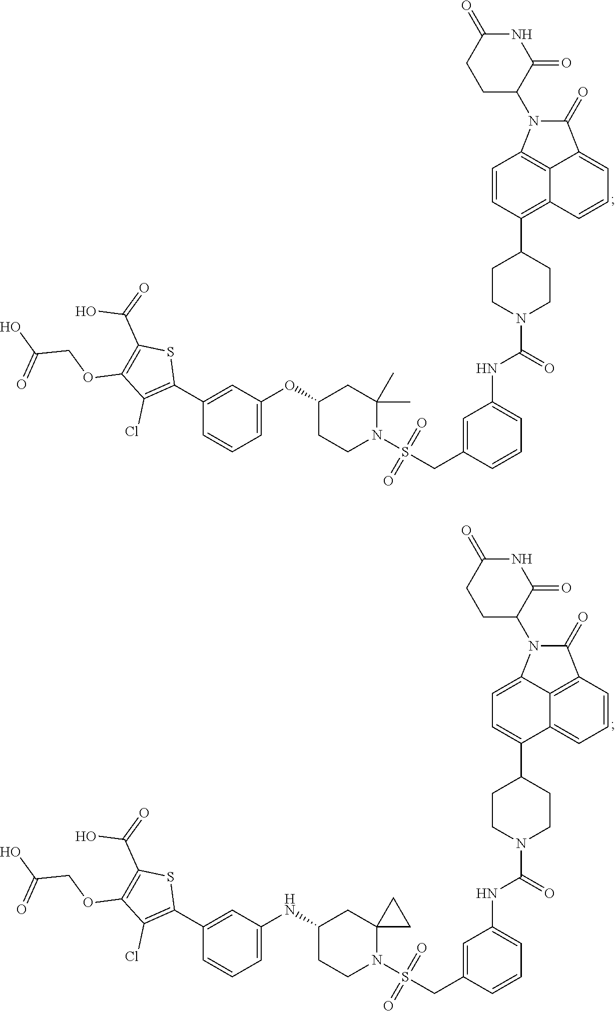 [SMILES: CC1(C)C[C@@H](Oc2cccc(-c3sc(C(=O)O)c(OCC(=O)O)c3Cl)c2)CCN1S(=O)(=O)Cc1cccc(NC(=O)N2CCC(c3ccc4c5c(cccc35)C(=O)N4C3CCC(=O)NC3=O)CC2)c1 ; O=C(O)COc1c(C(=O)O)sc(-c2cccc(N[C@H]3CCN(S(=O)(=O)Cc4cccc(NC(=O)N5CCC(c6ccc7c8c(cccc68)C(=O)N7C6CCC(=O)NC6=O)CC5)c4)C4(CC4)C3)c2)c1Cl] 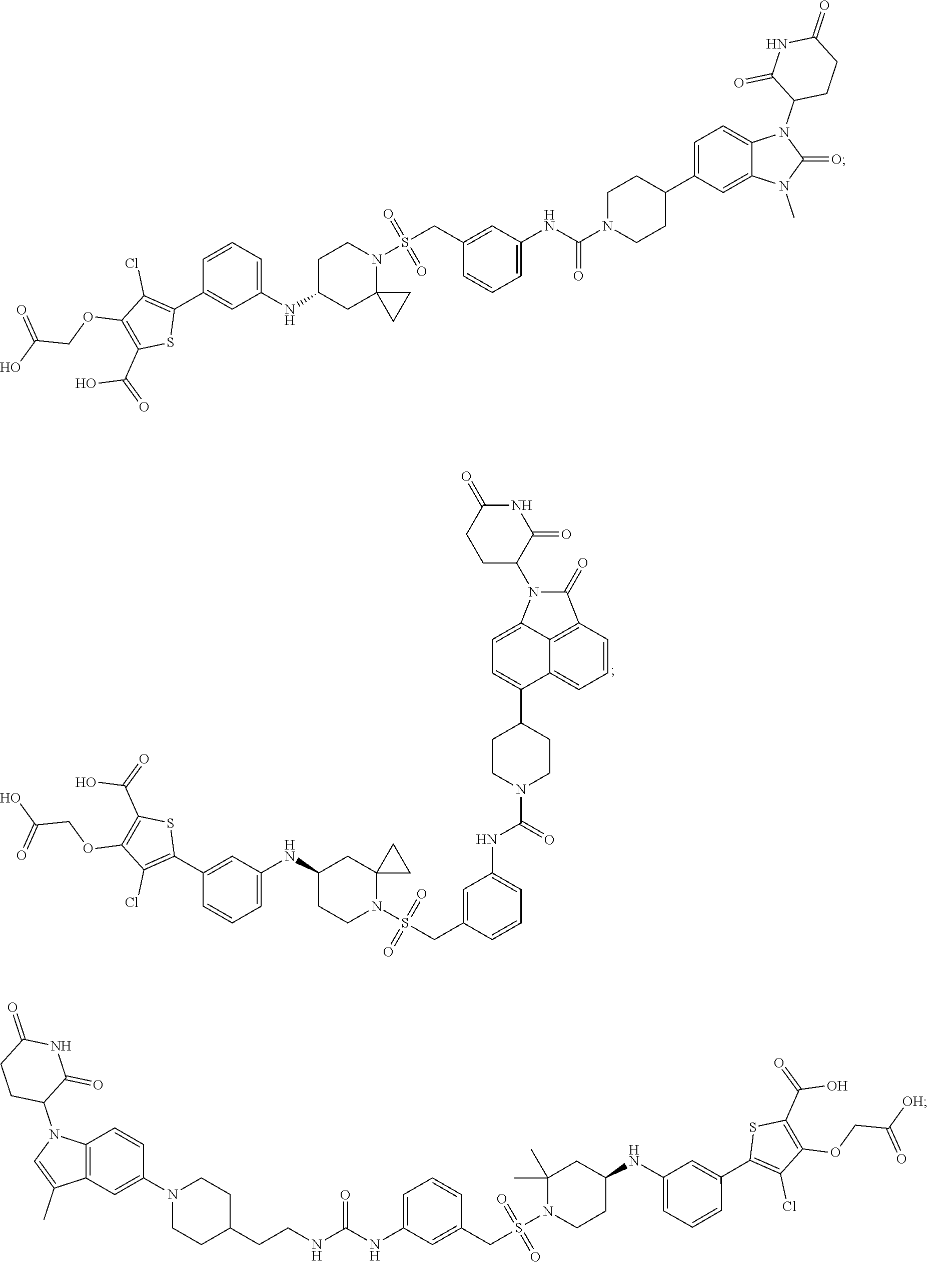 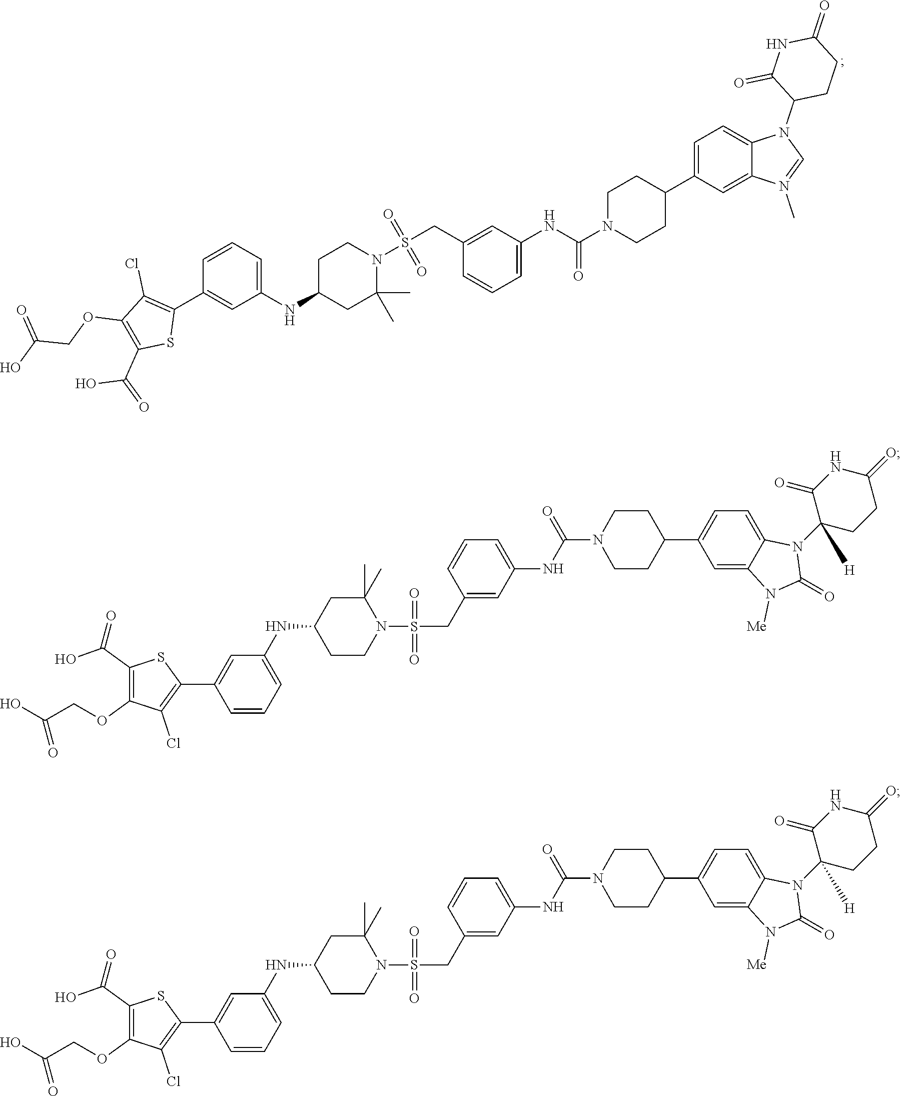 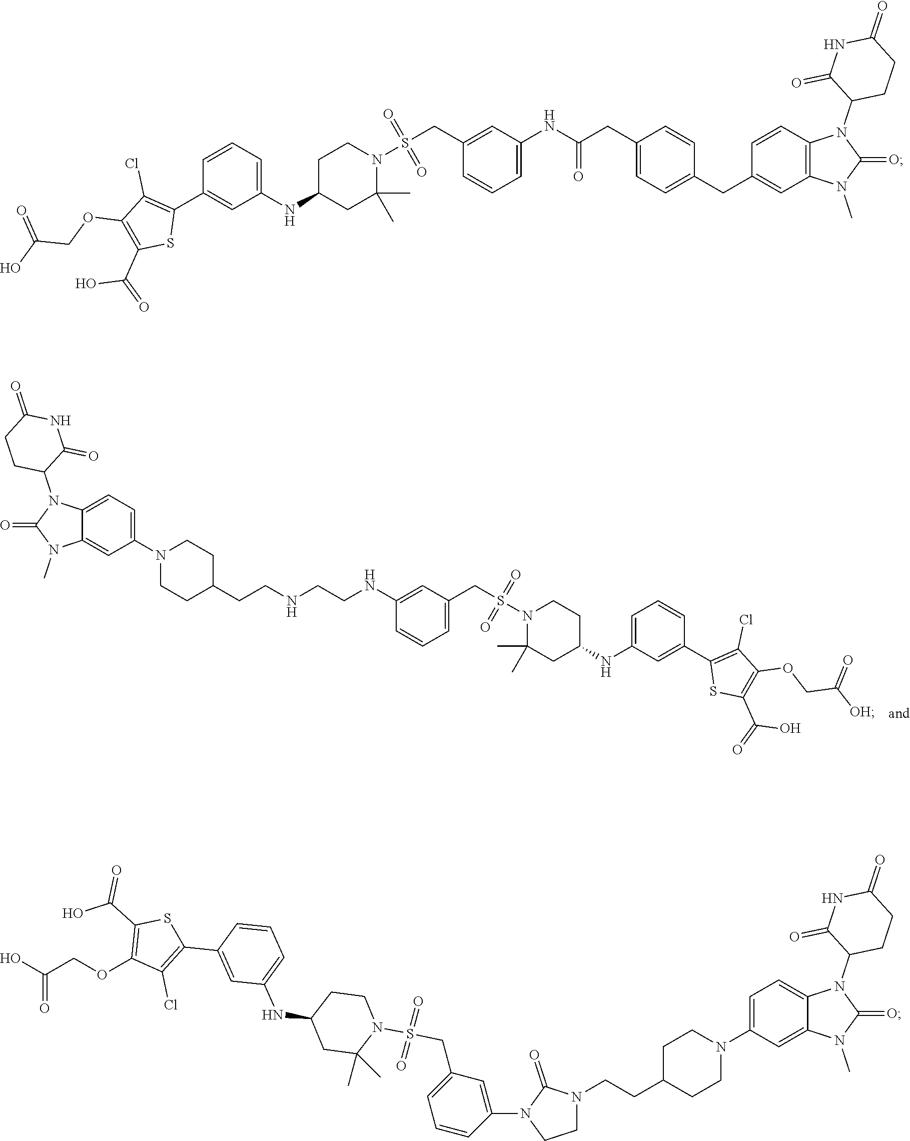
or a pharmaceutically acceptable salt thereof.

199. A pharmaceutical composition comprising a compound of claim 1, or a pharmaceutically acceptable salt thereof.

200. A method for treating cancer in a subject, the method comprising administering to the subject a therapeutically effective amount of a compound of claim 1 or a pharmaceutically acceptable salt thereof.

201. (canceled)

202. A method for decreasing the level of a protein in a mammalian cell, the method comprising contacting the mammalian cell with an effective amount of a compound of claim 1 or a pharmaceutically acceptable salt thereof; wherein the protein is PTPN1, PTPN2, or a combination thereof.

203-206. (canceled)

207. A method for treating a metabolic disease in a subject, the method comprising administering to the subject a therapeutically effective amount of a compound of claim 1 or a pharmaceutically acceptable salt thereof.

208. The method of claim 207, wherein the metabolic disease is NAFLD, NASH, type 2 diabetes, or a combination of any of the foregoing.

209-216. (canceled)


SEQUENCE LISTING (3 sequences)

SEQ ID NO: 1 - protein, 404 aa, synthetic construct
MAMPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDTENLYFQSHHHHHHHH

SEQ ID NO: 2 - protein, 13 aa, synthetic construct
  MOD_RES 1: note = (Oregon Green)-(NH-CH2-CH2-O-CH2-CH2-O-CH2-CO)-L-threonine
  MOD_RES 5: note = O-phospho-L-tyrosine
  MOD_RES 13: note = L-lysine-NH2
XRDIXETDYYRKX

SEQ ID NO: 3 - protein, 339 aa, synthetic construct
MAHHHHHHSSGLVPRGSHMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN